(19) United States
(12) Patent Application Publication
(10) Pub. No.: US 2026/0285853 A1
(43) Pub. Date: Sep. 24, 2026

(54) COMPOUNDS AND METHODS FOR MODULATING SPLICING

(71) Applicant: REMIX THERAPEUTICS INC., Watertown, MA (US)

(72) Inventors: Dominic Reynolds, Stoneham, MA (US); Benoît Moreau, Newton, MA (US); Sudeep Prajapati, Somerville, MA (US); Stepan Vyskocil, Benesov (CZ); Derek LaPlaca, Quincy, MA (US); Francois Brucelle, Belmont, MA (US)

(21) Appl. No.: 19/160,796

(22) PCT Filed: Mar. 1, 2024
(86) PCT No.: PCT/US2024/018234

§ 371 Date: Aug. 28, 2025

Related U.S. Application Data
(60) Provisional application No. 63/449,299, filed on Mar. 1, 2023.

(51) Int. Cl. C07D 471/04; A61K 31/435; A61K 31/4353; A61K 31/46; A61K 31/4985; A61P 35/00; C07D 401/14; C07D 471/08; C07D 487/04; C07D 519/00

(52) U.S. Cl. CPC C07D 471/04; A61K 31/435; A61K 31/4353; A61K 31/46; A61K 31/4985; A61P 35/00; C07D 401/14; C07D 471/08; C07D 487/04; C07D 519/00

(57) ABSTRACT

The present disclosure features compounds and related compositions that, inter alia, modulate nucleic acid splicing, e.g., splicing of a pre-mRNA, as well as methods of use thereof.

### CLAIM OF PRIORITY

This application claims priority to U.S. Patent Application No. 63/449,299, filed Mar. 1, 2023. The entire contents of the foregoing application are incorporated herein by reference.

### BACKGROUND

Alternative splicing is a major source of protein diversity in higher eukaryotes and is frequently regulated in a tissue-specific or development stage-specific manner. Disease associated alternative splicing patterns in pre-mRNAs are often mapped to changes in splice site signals or sequence motifs and regulatory splicing factors (Faustino and Cooper (2003), Genes Dev 17 (4): 419-37). Current therapies to modulate RNA expression involve oligonucleotide targeting and gene therapy; however, each of these modalities exhibit unique challenges as currently presented. As such, there is a need for new technologies to modulate RNA expression, including the development of small molecule compounds that target splicing.

### SUMMARY

The present disclosure features compounds and related compositions that, inter alia, modulate nucleic acid splicing, e.g., splicing of a pre-mRNA, as well as methods of use thereof. In an embodiment, the compounds described herein are compounds of Formula (I) or (II) (e.g., a compound of Formulas (I-a), (I-b), (I-c), (I-d), (I-e), (I-f), (I-g), (I-h), (I-i), (I-j), (II-a), (II-b), (II-c), (II-d), and (II-e)) and pharmaceutically acceptable salts, solvates, hydrates, tautomers, or stereoisomers thereof. The present disclosure additionally provides methods of using the compounds of the invention (e.g., compounds of Formulas (I-a), (I-b), (I-c), (I-d), (I-e), (I-f), (I-g), (I-h), (I-i), (I-j), (II-a), (II-b), (II-c), (II-d), and (II-e) and pharmaceutically acceptable salts, solvates, hydrates, tautomers, stereoisomers thereof), and compositions thereof, e.g., to target, and in embodiments bind or form a complex with, a nucleic acid (e.g., a pre-mRNA or nucleic acid component of a small nuclear ribonucleoprotein (snRNP) or spliceosome), a protein (e.g., a protein component of an snRNP or spliceosome, e.g., a member of the splicing machinery, e.g., one or more of the U1, U2, U4, U5, U6, U11, U12, U4atac, U6atac snRNPs), or a combination thereof. In another aspect, the compounds described herein may be used to alter the composition or structure of a nucleic acid (e.g., a pre-mRNA or mRNA (e.g., a pre-mRNA and the mRNA which arises from the pre-mRNA), e.g., by increasing or decreasing splicing at a splice site. In some embodiments, increasing or decreasing splicing results in modulating the level of a gene product (e.g., an RNA or protein) produced.

In another aspect, the compounds described herein may be used for the prevention and/or treatment of a disease, disorder, or condition, e.g., a disease, disorder or condition associated with splicing, e.g., alternative splicing. In some embodiments, the compounds described herein (e.g., compounds of Formulas (I-a), (I-b), (I-c), (I-d), (I-e), (I-f), (I-g), (I-h), (I-i), (I-j), (II-a), (II-b), (II-c), (II-d), and (II-e), and pharmaceutically acceptable salts, solvates, hydrates, tautomers, stereoisomers thereof) and compositions thereof are used for the prevention and/or treatment of a proliferative disease, disorder, or condition (e.g., a disease, disorder, or condition characterized by unwanted cell proliferation, e.g., a cancer or a benign neoplasm) in a subject. In some embodiments, the compounds described herein (e.g., compounds of Formulas (I-a), (I-b), (I-c), (I-d), (I-e), (I-f), (I-g), (I-h), (I-i), (I-j), (II-a), (II-b), (II-c), (II-d), and (II-e), and pharmaceutically acceptable salts, solvates, hydrates, tautomers, stereoisomers thereof) and compositions thereof are used for the prevention and/or treatment of a non-proliferative disease, disorder, or condition. In some embodiments, the compounds described herein (e.g., compounds of Formulas (I-a), (I-b), (I-c), (I-d), (I-e), (I-f), (I-g), (I-h), (II-a), (II-b), (II-c), (II-d), (II-e), (II-f), (II-g), (II-h), (II-i), (II-j), (II-k), (II-l), or (II-m), and pharmaceutically acceptable salts, solvates, hydrates, tautomers, stereoisomers thereof) and compositions thereof are used for the prevention and/or treatment of a neurological disease or disorder, an autoimmune disease or disorder, immunodeficiency disease or disorder, a lysosomal storage disease or disorder, a cardiovascular disease or disorder, a metabolic disease or disorder, a respiratory disease or disorder, a renal disease or disorder, or an infectious disease in a subject.

In another aspect, the present disclosure features a compound of Formula (I):

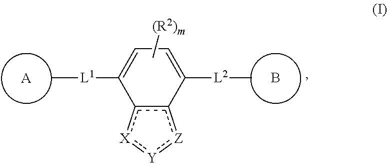

or a pharmaceutically acceptable salt, solvate, hydrate, tautomer, or stereoisomer thereof, wherein A, B, X, Y, Z, L¹, L², R², m, and subvariables thereof are as described herein.

In another aspect, the present disclosure features a compound of Formula (II):

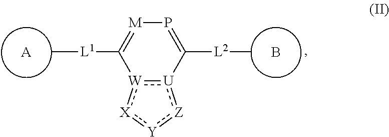

or a pharmaceutically acceptable salt, solvate, hydrate, tautomer, or stereoisomer thereof, wherein A, B, M, P, W, U, X, Y, Z, L¹, L², and subvariables thereof are as described herein.

In another aspect, the present invention provides pharmaceutical compositions comprising a compound of Formula (I) or (II) (e.g., a compound of Formulas (I-a), (I-b), (I-c), (I-d), (I-e), (I-f), (I-g), (I-h), (I-i), (I-j), (II-a), (II-b), (II-c), (II-d), and (II-e)), or a pharmaceutically acceptable salt, solvate, hydrate, tautomer, or stereoisomer thereof, and optionally a pharmaceutically acceptable excipient. In an embodiment, the pharmaceutical compositions described herein include an effective amount (e.g., a therapeutically effective amount) of a compound of Formula (I) or (II) (e.g., a compound of Formulas (I-a), (I-b), (I-c), (I-d), (I-e), (I-f), (I-g), (I-h), (I-i), (I-j), (II-a), (II-b), (II-c), (II-d), and (II-e)), or a pharmaceutically acceptable salt, solvate, hydrate, tautomer, or stereoisomer thereof.

In another aspect, the present disclosure provides methods for modulating splicing, e.g., splicing of a nucleic acid (e.g., a DNA or RNA, e.g., a pre-mRNA) with a compound of Formula (I) or (II) (e.g., a compound of Formulas (I-a), (I-b), (I-c), (I-d), (I-e), (I-f), (I-g), (I-h), (I-i), (I-j), (II-a), (II-b), (II-c), (II-d), and (II-e)) or a pharmaceutically acceptable salt, solvate, hydrate, tautomer, or stereoisomer thereof. In another aspect, the present disclosure provides compositions for use in modulating splicing, e.g., splicing of a nucleic acid (e.g., a DNA or RNA, e.g., a pre-mRNA) with a compound of Formula (I) or (II) (e.g., a compound of Formulas (I-a), (I-b), (I-c), (I-d), (I-e), (I-f), (I-g), (I-h), (I-i), (I-j), (II-a), (II-b), (II-c), (II-d), and (II-e)) or a pharmaceutically acceptable salt, solvate, hydrate, tautomer, or stereoisomer thereof. Modulation of splicing may comprise impacting any step involved in splicing and may include an event upstream or downstream of a splicing event. For example, in some embodiments, the compound of Formula (I) or (II) binds to a target, e.g., a target nucleic acid (e.g., DNA or RNA, e.g., a precursor RNA, e.g., a pre-mRNA), a target protein, or combination thereof (e.g., an snRNP and a pre-mRNA). A target may include a splice site in a pre-mRNA or a component of the splicing machinery, such as the U1 snRNP. In some embodiments, the compound of Formula (I) or (II) alters a target nucleic acid (e.g., DNA or RNA, e.g., a precursor RNA, e.g., a pre-mRNA), target protein, or combination thereof. In some embodiments, the compound of Formula (I) or (II) increases or decreases splicing at a splice site on a target nucleic acid (e.g., an RNA, e.g., a precursor RNA, e.g., a pre-mRNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formula (I) or (II), e.g., in a healthy or diseased cell or tissue). In some embodiments, the presence of a compound of Formula (I) or (II) results an increase or decrease of transcription of a target nucleic acid (e.g., an RNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formula (I) or (II), e.g., in a healthy or diseased cell or tissue).

In another aspect, the present disclosure provides methods for preventing and/or treating a disease, disorder, or condition in a subject by administering a compound of Formula (I) or (II) (e.g., a compound of Formulas (I-a), (I-b), (I-c), (I-d), (I-e), (I-f), (I-g), (I-h), (I-i), (I-j), (II-a), (II-b), (II-c), (II-d), and (II-e)) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides methods for treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides methods of down-regulating the expression of (e.g., the level of or the rate of production of) a target protein with a compound of Formula (I) or (II) (e.g., a compound of Formulas (I-a), (I-b), (I-c), (I-d), (I-e), (I-f), (I-g), (I-h), (I-i), (I-j), (II-a), (II-b), (II-c), (II-d), and (II-e)) or a pharmaceutically acceptable salt, solvate, hydrate, tautomer, or stereoisomer thereof in a biological sample or subject. In another aspect, the present disclosure provides methods of up-regulating the expression of (e.g., the level of or the rate of production of) a target protein with a compound of Formula (I) or (II) (e.g., a compound of Formulas (I-a), (I-b), (I-c), (I-d), (I-e), (I-f), (I-g), (I-h), (I-i), (I-j), (II-a), (II-b), (II-c), (II-d), and (II-e)) or a pharmaceutically acceptable salt, solvate, hydrate, tautomer, or stereoisomer thereof in a biological sample or subject. In another aspect, the present disclosure provides methods of altering the isoform of a target protein with a compound of Formula (I) or (II) (e.g., a compound of Formulas (I-a), (I-b), (I-c), (I-d), (I-e), (I-f), (I-g), (I-h), (I-i), (I-j), (II-a), (II-b), (II-c), (II-d), and (II-e)) or a pharmaceutically acceptable salt, solvate, hydrate, tautomer, or stereoisomer thereof in a biological sample or subject. Another aspect of the disclosure relates to methods of inhibiting the activity of a target protein in a biological sample or subject. In some embodiments, administration of a compound of Formula (I) or (II) to a biological sample, a cell, or a subject comprises inhibition of cell growth or induction of cell death.

In another aspect, the present disclosure provides compositions for use in preventing and/or treating a disease, disorder, or condition in a subject by administering a compound of Formula (I) or (II) (e.g., a compound of Formulas (I-a), (I-b), (I-c), (I-d), (I-e), (I-f), (I-g), (I-h), (I-i), (I-j), (II-a), (II-b), (II-c), (II-d), and (II-e)) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides methods for treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides compositions for use in down-regulating the expression of (e.g., the level of or the rate of production of) a target protein with a compound of Formula (I) or (II) (e.g., a compound of Formulas (I-a), (I-b), (I-c), (I-d), (I-e), (I-f), (I-g), (I-h), (I-i), (I-j), (II-a), (II-b), (II-c), (II-d), and (II-e)) or a pharmaceutically acceptable salt, solvate, hydrate, tautomer, or stereoisomer thereof in a biological sample or subject. In another aspect, the present disclosure provides compositions for use in up-regulating the expression of (e.g., the level of or the rate of production of) a target protein with a compound of Formula (I) or (II) (e.g., a compound of Formulas (I-a), (I-b), (I-c), (I-d), (I-e), (I-f), (I-g), (I-h), (I-i), (I-j), (II-a), (II-b), (II-c), (II-d), and (II-e)) or a pharmaceutically acceptable salt, solvate, hydrate, tautomer, or stereoisomer thereof in a biological sample or subject. In another aspect, the present disclosure provides compositions for use in altering the isoform of a target protein with a compound of Formula (I) or (II) (e.g., a compound of Formulas (I-a), (I-b), (I-c), (I-d), (I-e), (I-f), (I-g), (I-h), (I-i), (I-j), (II-a), (II-b), (II-c), (II-d), and (II-e)) or a pharmaceutically acceptable salt, solvate, hydrate, tautomer, or stereoisomer thereof in a biological sample or subject. Another aspect of the disclosure relates to compositions for use in inhibiting the activity of a target protein in a biological sample or subject. In some embodiments, administration of a compound of Formula (I) or (II) to a biological sample, a cell, or a subject comprises inhibition of cell growth or induction of cell death.

In another aspect, the present disclosure features kits comprising a container with a compound of Formula (I) or (II) (e.g., a compound of Formulas (I-a), (I-b), (I-c), (I-d), (I-e), (I-f), (I-g), (I-h), (I-i), (I-j), (II-a), (II-b), (II-c), (II-d), and (II-e)), or a pharmaceutically acceptable salt, solvate, hydrate, tautomer, stereoisomer thereof, or a pharmaceutical composition thereof. In certain embodiments, the kits described herein further include instructions for administering the compound of Formula (I) or (II) or the pharmaceutically acceptable salt, solvate, hydrate, tautomer, stereoisomer thereof, or the pharmaceutical composition thereof.

The details of one or more embodiments of the invention are set forth herein. Other features, objects, and advantages of the invention will be apparent from the Detailed Description, the Examples, and the Claims.

### DETAILED DESCRIPTION

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 2 to 6 carbon atoms (“C₂-C₆ alkyl”). In some embodiments, an alkyl group has 1 carbon atom (“C₁ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₆ alkyl.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₁-C₁₀ alkenyl. In certain embodiments, the alkenyl group is substituted C₂-C₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the term “haloalkyl,” refers to a non-cyclic stable straight or branched chain, or combinations thereof, including at least one carbon atom and at least one halogen selected from the group consisting of F, Cl, Br, and I. The halogen(s) F, Cl, Br, and I may be placed at any position of the haloalkyl group. Exemplary haloalkyl groups include, but are not limited to: —CF₃, —CCl₃, —CH₂—CF₃, —CH₂—CCl₃, —CH₂—CBr₃, —CH₂—Cl₃, —CH₂—CH₂—CH(CF₃)—CH₃, —CH₂—CH₂—CH(Br)—CH₃, and —CH₂—CH═CH—CH₂—CF₃. Each instance of a haloalkyl group may be independently optionally substituted, i.e., unsubstituted (an “unsubstituted haloalkyl”) or substituted (a “substituted haloalkyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “heteroaryl” refers to a radical of a 5-10 membered monocyclic or bicyclic 4n+2 aromatic ring system (e.g., having 6 or 10π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety. Each instance of a heteroaryl group may be independently optionally substituted, i.e., unsubstituted (an “unsubstituted heteroaryl”) or substituted (a “substituted heteroaryl”) with one or more substituents e.g., for instance from 1 to 5 substituents, 1 to 3 substituents, or 1 substituent.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

“Heterocyclyl” as used herein refers to a radical of a 3- to 16-membered non-aromatic ring system having ring carbon atoms and 1 to 8 ring heteroatoms, wherein each heteroatom is independently selected from nitrogen, oxygen, sulfur, boron, phosphorus, and silicon (“3-16 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-16 membered heterocyclyl. In certain embodiments, the heterocyclyl group is substituted 3-16 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin2-onyl), and thianyl. Exemplary 6-membered heterocyclyl groups containing two heteroatoms include, without limitation, piperazinyl, morpholinyl, pyridazinonyl(2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

The terms “alkylene,” “alkenylene,” “alkynylene,” “haloalkylene,” “heteroalkylene,” “cycloalkylene,” or “heterocyclylene,” alone or as part of another substituent, mean, unless otherwise stated, a divalent radical derived from an alkyl, alkenyl, alkynyl, haloalkylene, heteroalkylene, cycloalkyl, or heterocyclyl respectively. For example, the term “alkenylene,” by itself or as part of another substituent, means, unless otherwise stated, a divalent radical derived from an alkene. An alkylene, alkenylene, alkynylene, haloalkylene, heteroalkylene, cycloalkylene, or heterocyclylene group may be described as, e.g., a C₁-C₆-membered alkylene, C₂-C₆-membered alkenylene, C₂-C₆-membered alkynylene, C₁-C₆-membered haloalkylene, C₁-C₆-membered heteroalkylene, C₃-C₈-membered cycloalkylene, or C₃-C₈-membered heterocyclylene, wherein the term “membered” refers to the non-hydrogen atoms within the moiety. In the case of heteroalkylene and heterocyclylene groups, heteroatoms can also occupy either or both of the chain termini (e.g., alkyleneoxy, alkylenedioxy, alkyleneamino, alkylenediamino, and the like). Still further, no orientation of the linking group is implied by the direction in which the formula of the linking group is written. For example, the formula —C(O)₂R′— may represent both —C(O)₂R′— and —R′C(O)₂—.

As used herein, the terms “cyano” or “—CN” refer to a substituent having a carbon atom joined to a nitrogen atom by a triple bond, e.g., C≡N.

As used herein, the terms “halogen” or “halo” refer to fluorine, chlorine, bromine or iodine.

As used herein, the term “hydroxy” refers to —OH.

As used herein, the term “nitro” refers to a substitutent having two oxygen atoms bound to a nitrogen atom, e.g., —NO₂.

As used herein, the term “nucleobase” as used herein, is a nitrogen-containing biological compounds found linked to a sugar within a nucleoside—the basic building blocks of deoxyribonucleic acid (DNA) and ribonucleic acid (RNA). The primary, or naturally occurring, nucleobases are cytosine (DNA and RNA), guanine (DNA and RNA), adenine (DNA and RNA), thymine (DNA) and uracil (RNA), abbreviated as C, G, A, T, and U, respectively. Because A, G, C, and T appear in the DNA, these molecules are called DNA-bases; A, G, C, and U are called RNA-bases. Adenine and guanine belong to the double-ringed class of molecules called purines (abbreviated as R). Cytosine, thymine, and uracil are all pyrimidines. Other nucleobases that do not function as normal parts of the genetic code, are termed non-naturally occurring. In an embodiment, a nucleobase may be chemically modified, for example, with an alkyl (e.g., methyl), halo, —O-alkyl, or other modification.

As used herein, the term “nucleic acid” refers to deoxyribonucleic acids (DNA) or ribonucleic acids (RNA) and polymers thereof in either single- or double-stranded form. The term “nucleic acid” includes a gene, cDNA, pre-mRNA, or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementarity sequences as well as the sequence explicitly indicated.

As used herein, “oxo” refers to a carbonyl, i.e., —C(O)—.

The symbol “ [symbol] ” as used herein in relation to a compound of Formula (I) or (II) refers to an attachment point to another moiety or functional group within the compound.

Alkyl, alkenyl, alkynyl, haloalkyl, heteroalkyl, cycloalkyl, heterocyclyl, aryl, and heteroaryl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

The compounds provided herein may exist in one or more particular geometric, optical, enantiomeric, diasteriomeric, epimeric, stereoisomeric, tautomeric, conformational, or anomeric forms, including but not limited to: cis- and trans-forms; E- and Z-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and half chair-forms; and combinations thereof, hereinafter collectively referred to as “isomers” (or “isomeric forms”).

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. In an embodiment, the stereochemistry depicted in a compound is relative rather than absolute. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). This disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising an enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising an enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound.

In some embodiments, a diastereomerically pure compound can be present with other active or inactive ingredients. For example, a pharmaceutical composition comprising a diastereomerically pure exo compound can comprise, for example, about 90% excipient and about 10% diastereomerically pure exo compound. In certain embodiments, the diastereomerically pure exo compound in such compositions can, for example, comprise, at least about 95% by weight exo compound and at most about 5% by weight endo compound, by total weight of the compound. For example, a pharmaceutical composition comprising a diastereomerically pure endo compound can comprise, for example, about 90% excipient and about 10% diastereomerically pure endo compound. In certain embodiments, the diastereomerically pure endo compound in such compositions can, for example, comprise, at least about 95% by weight endo compound and at most about 5% by weight exo compound, by total weight of the compound.

In some embodiments, an isomerically pure compound can be present with other active or inactive ingredients. For example, a pharmaceutical composition comprising a isomerically pure exo compound can comprise, for example, about 90% excipient and about 10% isomerically pure exo compound. In certain embodiments, the isomerically pure exo compound in such compositions can, for example, comprise, at least about 95% by weight exo compound and at most about 5% by weight endo compound, by total weight of the compound. For example, a pharmaceutical composition comprising an isomerically pure endo compound can comprise, for example, about 90% excipient and about 10% isomerically pure endo compound. In certain embodiments, the isomerically pure endo compound in such compositions can, for example, comprise, at least about 95% by weight endo compound and at most about 5% by weight exo compound, by total weight of the compound.

In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; N may be in any isotopic form, including ¹⁴N and ¹⁵N; F may be in any isotopic form, including ¹⁸F, ¹⁹F, and the like.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66:1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

In addition to salt forms, the present disclosure provides compounds in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I) or (II) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomer” refers to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base. Tautomeric forms may be relevant to the attainment of the optimal chemical reactivity and biological activity of a compound of interest.

### Other Definitions

The following definitions are more general terms used throughout the present disclosure.

The articles “a” and “an” refer to one or more than one (e.g., to at least one) of the grammatical object of the article. By way of example, “an element” means one element or more than one element. The term “and/or” means either “and” or “or” unless indicated otherwise.

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., mass spectrometer to acquire mass spectrometry data.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “condition,” “disease,” and “disorder” are used interchangeably.

An “effective amount” of a compound of Formula (I) or (II) refers to an amount sufficient to elicit the desired biological response, i.e., treating the condition. As will be appreciated by those of ordinary skill in this art, the effective amount of a compound of Formula (I) or (II) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formula (I) or (II) is an amount sufficient to provide a therapeutic benefit in the treatment of a condition or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprised therein. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

“Prevention,” “prevent,” and “preventing” as used herein refers to a treatment that comprises administering a therapy, e.g., administering a compound described herein (e.g., a compound of Formula (I) or (II)) prior to the onset of a disease, disorder, or condition in order to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

As used herein, the terms “treatment,” “treat,” and “treating” refer to reversing, alleviating, delaying the onset of, or inhibiting the progress of one or more of a symptom, manifestation, or underlying cause of a disease, disorder, or condition (e.g., as described herein), e.g., by administering a therapy, e.g., administering a compound described herein (e.g., a compound of Formula (I) or (II)). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

A “proliferative disease” refers to a disease that occurs due to abnormal extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); 4) the pathological angiogenesis as in proliferative retinopathy and tumor metastasis; or 5) evasion of host immune surveillance and elimination of neoplastic cells. Exemplary proliferative diseases include cancers (i.e., “malignant neoplasms”), benign neoplasms, and angiogenesis.

A “non-proliferative disease” refers to a disease that does not primarily extend through the abnormal multiplication of cells. A non-proliferative disease may be associated with any cell type or tissue type in a subject. Exemplary non-proliferative diseases include neurological diseases or disorders (e.g., a repeat expansion disease); autoimmune disease or disorders; immunodeficiency diseases or disorders; lysosomal storage diseases or disorders; inflammatory diseases or disorders; cardiovascular conditions, diseases, or disorders; metabolic diseases or disorders; respiratory conditions, diseases, or disorders; renal diseases or disorders; and infectious diseases.

### Compounds

The present disclosure features a compound of Formula (I):

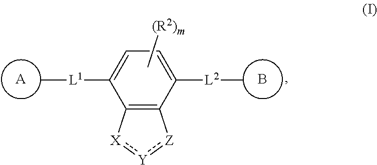

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; X, Y, and Z are each independently C(R^{3a}), C(R^{3a})(R^{3b}), N, N(R^{3c}), S, or O, wherein at least one of X, Y, and Z is N, N(R^{3c}), or O, and the bonds in the ring comprising X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} and R^{3b} are each independently hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}; or each of R^{3a} and R^{3b} together with the carbon atom to which they are attached, form an oxo group; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In another aspect, the present disclosure features a compound of Formula (II):

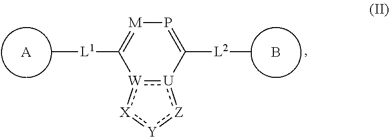

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; M and P are each independently C(R²) or N; U and W are each independently C or N; X, Y, and Z are each independently C(R^{3a}), N, N(R^{3c}), O, or S, wherein the bonds in the ring comprising U, W, X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶, or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸; each R⁴ is independently hydrogen, C₁-C₆-alkyl, C₁-C₆-heteroalkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —NR^{B}N(R^{B})(R^{C}), —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}, wherein C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, and C₁-C₆ alkylene-heteroaryl is optionally substituted with one or more R⁷; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}, wherein C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, and C₁-C₆ alkylene-heteroaryl is optionally substituted with one or more R⁷; or R^{B} and R^{C} together with the atom to which they are attached form a 3-7-membered heterocyclyl ring optionally substituted with one or more R⁷; each R^{D} is independently hydrogen, C₁-C₆ alkyl, C₂—C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}, each R^{A1} is hydrogen or C₁-C₆-alkyl; and x is 0, 1, or 2.

As generally described herein for Formulas (I) and (II), A and B, are each independently cycloalkyl, heterocyclyl, aryl, or heteroaryl, each of which is optionally substituted with one or more R¹.

In some embodiments, each of A and B are independently a monocyclic ring, e.g., monocyclic cycloalkyl, monocyclic heterocyclyl, monocyclic aryl, or monocyclic heteroaryl. The monocyclic ring may be saturated, partially unsaturated, or fully unsaturated (e.g., aromatic). In some embodiments, A or B are independently a monocyclic ring comprising between 3 and 10 ring atoms (e.g., 3, 4, 5, 6, 7, 8, 9, or 10 ring atoms). In some embodiments, A is a 4-membered monocyclic ring. In some embodiments, B is a 4-membered monocyclic ring. In some embodiments, A is a 5-membered monocyclic ring. In some embodiments, B is a 5-membered monocyclic ring. In some embodiments, A is a 6-membered monocyclic ring. In some embodiments, B is a 6-membered monocyclic ring. In some embodiments, A is a 7-membered monocyclic ring. In some embodiments, B is a 7-membered monocyclic ring. In some embodiments, A is an 8-membered monocyclic ring. In some embodiments, B is an 8-membered monocyclic ring. In some embodiments, A or B are independently a monocyclic ring optionally substituted with one or more R¹.

In some embodiments, A or B are independently a bicyclic ring, e.g., bicyclic cycloalkyl, bicyclic heterocyclyl, bicyclic aryl, or bicyclic heteroaryl. The bicyclic ring may be saturated, partially unsaturated, or fully unsaturated (e.g., aromatic). In some embodiments, A or B are independently a bicyclic ring comprising a fused, bridged, or spiro ring system. In some embodiments, A or B are independently a bicyclic ring comprising between 4 and 18 ring atoms (e.g., 4, 5, 6, 7, 8, 9, 10, 11, 12, 13, 14, 15, 16, 17, or 18 ring atoms). In some embodiments, A is a 6-membered bicyclic ring. In some embodiments, B is a 6-membered bicyclic ring. In some embodiments, A is a 7-membered bicyclic ring. In some embodiments, B is a 7-membered bicyclic ring. In some embodiments, A is an 8-membered bicyclic ring. In some embodiments, B is an 8-membered bicyclic ring. In some embodiments, A is a 9-membered bicyclic ring. In some embodiments, B is a 9-membered bicyclic ring. In some embodiments, A is a 10-membered bicyclic ring. In some embodiments, B is a 10-membered bicyclic ring. In some embodiments, A is an 11-membered bicyclic ring. In some embodiments, B is an 11-membered bicyclic ring. In some embodiments, A is a 12-membered bicyclic ring. In some embodiments, B is a 12-membered bicyclic ring. In some embodiments, A or B are independently a bicyclic ring optionally substituted with one or more R¹.

In some embodiments, A or B are independently a tricyclic ring, e.g., tricyclic cycloalkyl, tricyclic heterocyclyl, tricyclic aryl, or tricyclic heteroaryl. The tricyclic ring may be saturated, partially unsaturated, or fully unsaturated (e.g., aromatic). In some embodiments, A or B are independently a tricyclic ring that comprises a fused, bridged, or spiro ring system, or a combination thereof. In some embodiments, A or B are independently a tricyclic ring comprising between 6 and 24 ring atoms (e.g., 6, 7, 8, 9, 10, 11, 12, 13, 14, 15, 16, 17, 18, 19, 20, 21, 22, 23, or 24 ring atoms). In some embodiments, A is an 8-membered tricyclic ring. In some embodiments, B is an 8-membered tricyclic ring. In some embodiments, A is a 9-membered tricyclic ring. In some embodiments, B is a 9-membered tricyclic ring. In some embodiments, A is a 10-membered tricyclic ring. In some embodiments, B is a 10-membered tricyclic ring. In some embodiments, A or B are independently a tricyclic ring optionally substituted with one or more R¹.

In some embodiments, A or B are independently monocyclic cycloalkyl, monocyclic heterocyclyl, monocyclic aryl, or monocyclic heteroaryl. In some embodiments, A or B are independently bicyclic cycloalkyl, bicyclic heterocyclyl, bicyclic aryl, or bicyclic heteroaryl. In some embodiments, A or B are independently tricyclic cycloalkyl, tricyclic heterocyclyl, tricyclic aryl, or tricyclic heteroaryl. In some embodiments, A is monocyclic heterocyclyl. In some embodiments, B is monocyclic heterocyclyl. In some embodiments, A is bicyclic heterocyclyl. In some embodiments, B is bicyclic heterocyclyl. In some embodiments, A is monocyclic heteroaryl. In some embodiments, B is monocyclic heteroaryl. In some embodiments, A is bicyclic heteroaryl. In some embodiments, B is bicyclic heteroaryl.

In some embodiments, A or B are independently a nitrogen-containing heterocyclyl, e.g., heterocyclyl comprising one or more nitrogen atom. The one or more nitrogen atom of the nitrogen-containing heterocyclyl may be at any position of the ring. In some embodiments, the nitrogen-containing heterocyclyl is monocyclic, bicyclic, or tricyclic. In some embodiments, A or B are independently heterocyclyl comprising at least 1, at least 2, at least 3, at least 4, at least 5, or at least 6 nitrogen atoms. In some embodiments, A is heterocyclyl comprising 1 nitrogen atom. In some embodiments, B is heterocyclyl comprising 1 nitrogen atom. In some embodiments, A is heterocyclyl comprising 2 nitrogen atoms. In some embodiments, B is heterocyclyl comprising 2 nitrogen atoms. In some embodiments, A is heterocyclyl comprising 3 nitrogen atoms. In some embodiments, B is heterocyclyl comprising 3 nitrogen atoms. In some embodiments, A is heterocyclyl comprising 4 nitrogen atoms. In some embodiments, B is heterocyclyl comprising 4 nitrogen atoms. In some embodiments, A or B are independently a nitrogen-containing heterocyclyl comprising one or more additional heteroatoms, e.g., one or more of oxygen, sulfur, boron, silicon, or phosphorus. In some embodiments, the one or more nitrogen of the nitrogen-containing heterocyclyl is substituted, e.g., with R¹.

In some embodiments, A or B are independently a nitrogen-containing heteroaryl, e.g., heteroaryl comprising one or more nitrogen atom. The one or more nitrogen atom of the nitrogen-containing heteroaryl may be at any position of the ring. In some embodiments, the nitrogen-containing heteroaryl is monocyclic, bicyclic, or tricyclic. In some embodiments, A or B are independently heteroaryl comprising at least 1, at least 2, at least 3, at least 4, at least 5, or at least 6 nitrogen atoms. In some embodiments, A is heteroaryl comprising 1 nitrogen atom. In some embodiments, B is heteroaryl comprising 1 nitrogen atom. In some embodiments, A is heteroaryl comprising 2 nitrogen atoms. In some embodiments, B is heteroaryl comprising 2 nitrogen atoms. In some embodiments, A is heteroaryl comprising 3 nitrogen atoms. In some embodiments, B is heteroaryl comprising 3 nitrogen atoms. In some embodiments, A is heteroaryl comprising 4 nitrogen atoms. In some embodiments, B is heteroaryl comprising 4 nitrogen atoms. In some embodiments, A or B are independently a nitrogen-containing heteroaryl comprising one or more additional heteroatoms, e.g., one or more of oxygen, sulfur, boron, silicon, or phosphorus. In some embodiments, the one or more nitrogen of the nitrogen-containing heteroaryl is substituted, e.g., with R¹.

In some embodiments, A is a 6-membered nitrogen-containing heterocyclyl, e.g., a 6-membered heterocyclyl comprising one or more nitrogen. In some embodiments, A is a 6-membered heterocyclyl comprising 1 nitrogen atom. In some embodiments, A is a 6-membered heterocyclyl comprising 2 nitrogen atoms. In some embodiments, A is a 6-membered heterocyclyl comprising 3 nitrogen atoms. In some embodiments, A is a 6-membered heterocyclyl comprising 4 nitrogen atoms. The one or more nitrogen atom of the 6-membered nitrogen-containing heterocyclyl may be at any position of the ring. In some embodiments, A is a 6-membered nitrogen-containing heterocyclyl optionally substituted with one or more R¹. In some embodiments, the one or more nitrogen of the 6-membered nitrogen-containing heterocyclyl is substituted, e.g., with R¹. In some embodiments, A is a 6-membered nitrogen-containing heterocyclyl comprising one or more additional heteroatoms, e.g., one or more of oxygen, sulfur, boron, silicon, or phosphorus.

In some embodiments, B is a 5-membered nitrogen-containing heterocyclyl or heteroaryl, e.g., a 5-membered heterocyclyl or heteroaryl comprising one or more nitrogen. In some embodiments, B is a 5-membered heterocyclyl comprising 1 nitrogen atom. In some embodiments, B is a 5-membered heteroaryl comprising 1 nitrogen atom. In some embodiments, B is a 5-membered heterocyclyl comprising 2 nitrogen atoms. In some embodiments, B is a 5-membered heteroaryl comprising 2 nitrogen atoms. In some embodiments, B is a 5-membered heterocyclyl comprising 3 nitrogen atoms. In some embodiments, B is a 5-membered heteroaryl comprising 3 nitrogen atoms. The one or more nitrogen atom of the 5-membered nitrogen-containing heterocyclyl or heteroaryl may be at any position of the ring. In some embodiments, B is a 5-membered nitrogen-containing heterocyclyl optionally substituted with one or more R¹. In some embodiments, B is a 5-membered nitrogen-containing heteroaryl optionally substituted with one or more R¹. In some embodiments, the one or more nitrogen of the 5-membered nitrogen-containing heterocyclyl or heteroaryl is substituted, e.g., with R¹. In some embodiments, B is a 5-membered nitrogen-containing heterocyclyl or heteroaryl comprising one or more additional heteroatoms, e.g., one or more of oxygen, sulfur, boron, silicon, or phosphorus.

In some embodiments, B is a nitrogen-containing bicyclic heteroaryl (e.g., a 9-membered nitrogen-containing bicyclic heteroaryl), that is optionally substituted with one or more R¹. In some embodiments, B is a 9-membered bicyclic heteroaryl comprising 1 nitrogen atom. In some embodiments, B is a 9-membered bicyclic heteroaryl comprising 2 nitrogen atoms. In some embodiments, B is a 9-membered bicyclic heteroaryl comprising 3 nitrogen atoms. In some embodiments, B is a 9-membered bicyclic heteroaryl comprising 4 nitrogen atoms. The one or more nitrogen atom of the 9-membered bicyclic heteroaryl may be at any position of the ring. In some embodiments, B is a 9-membered bicyclic heteroaryl substituted with one or more R¹.

In some embodiments, each of A and B are independently selected from:

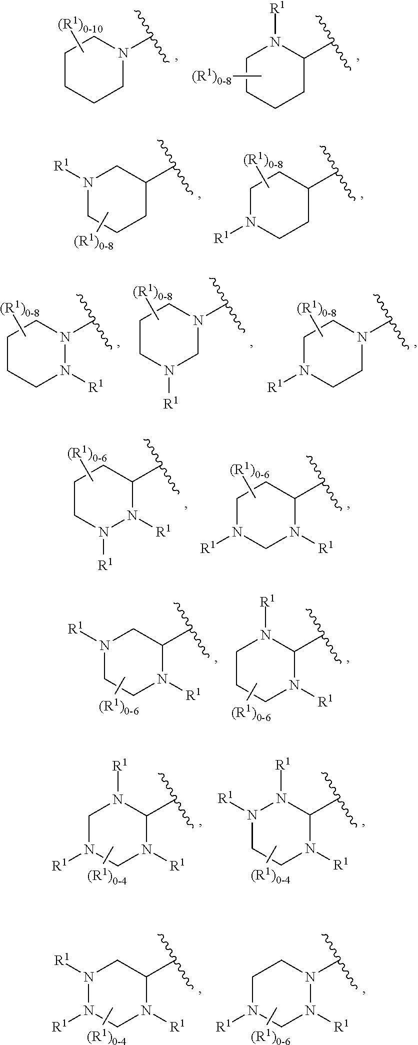

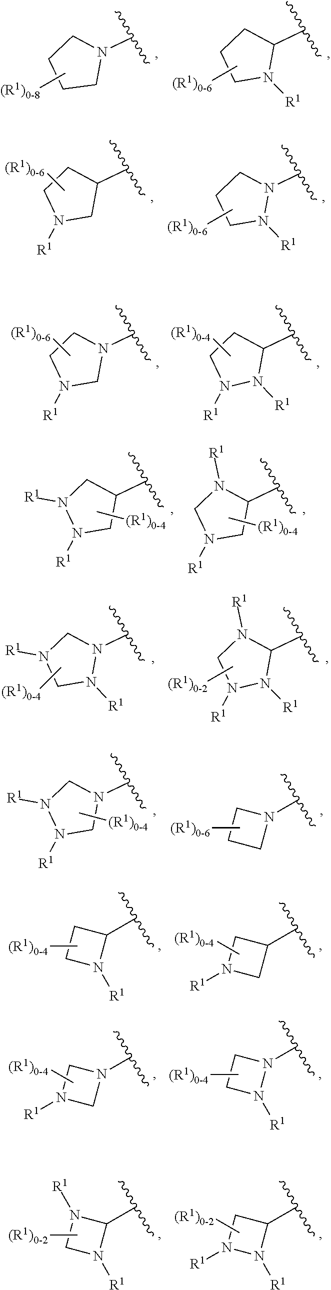

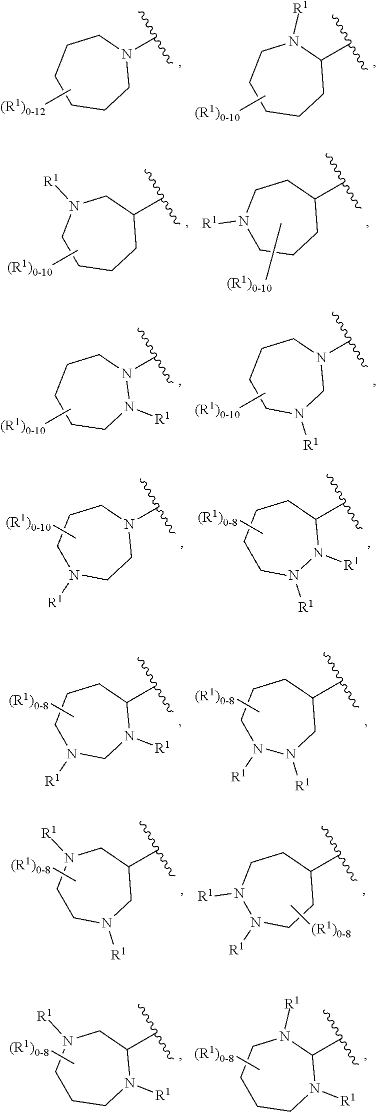

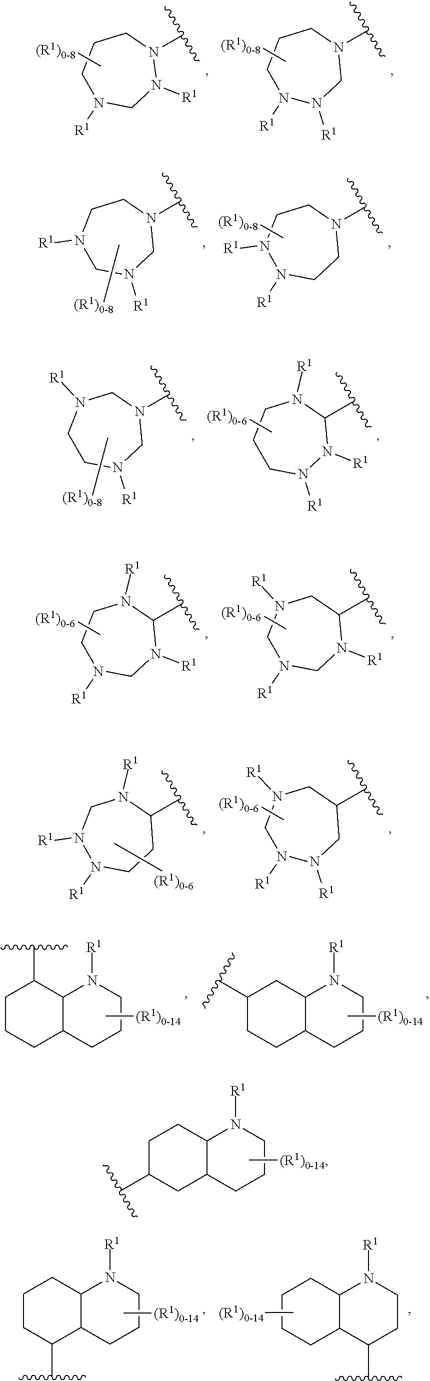

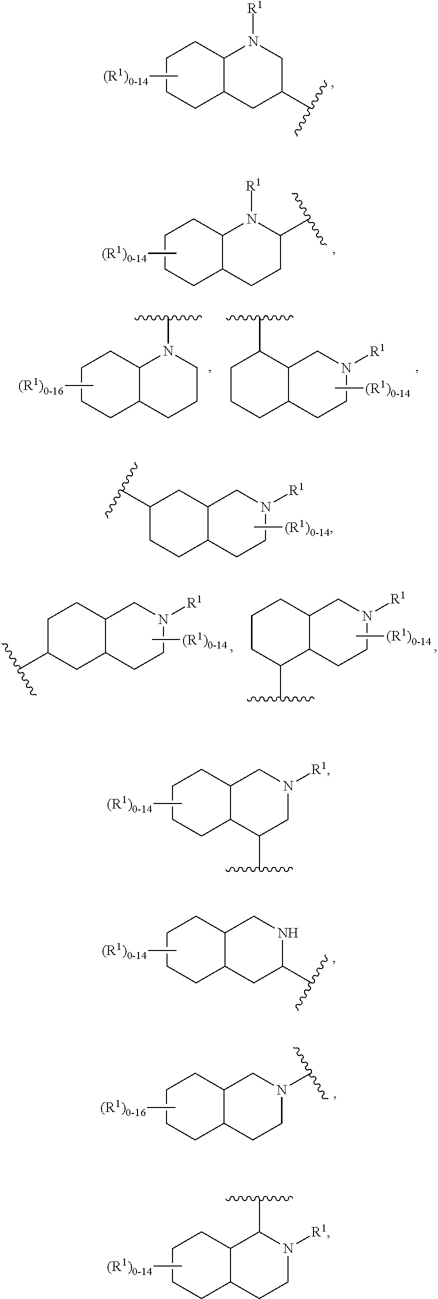

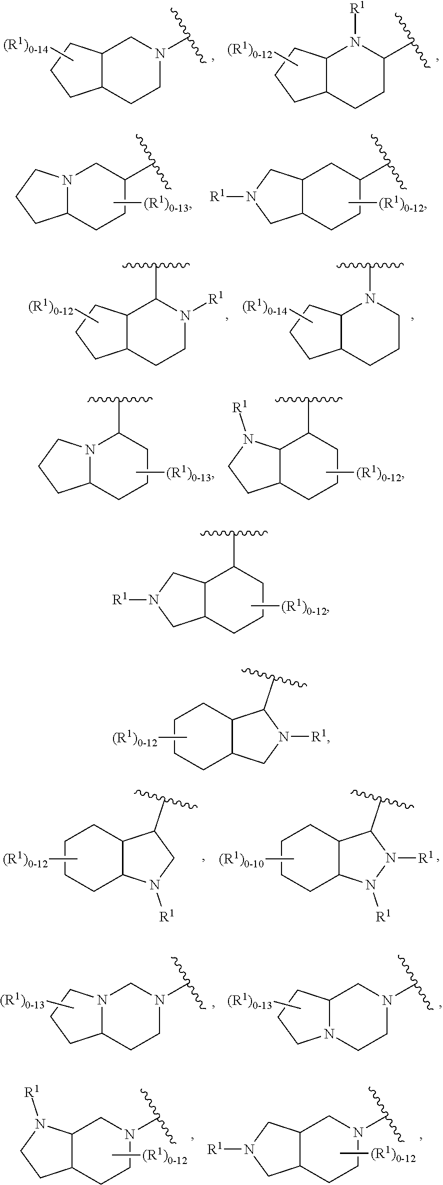

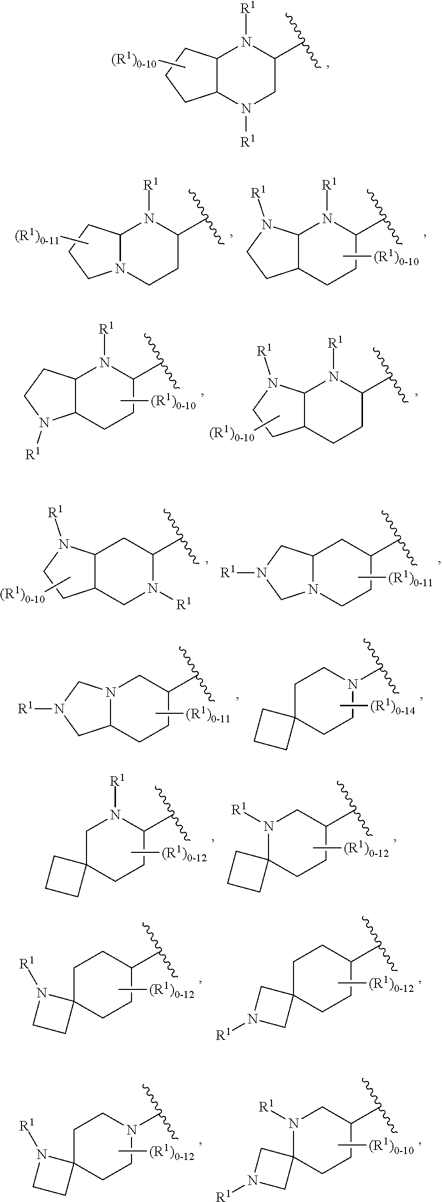

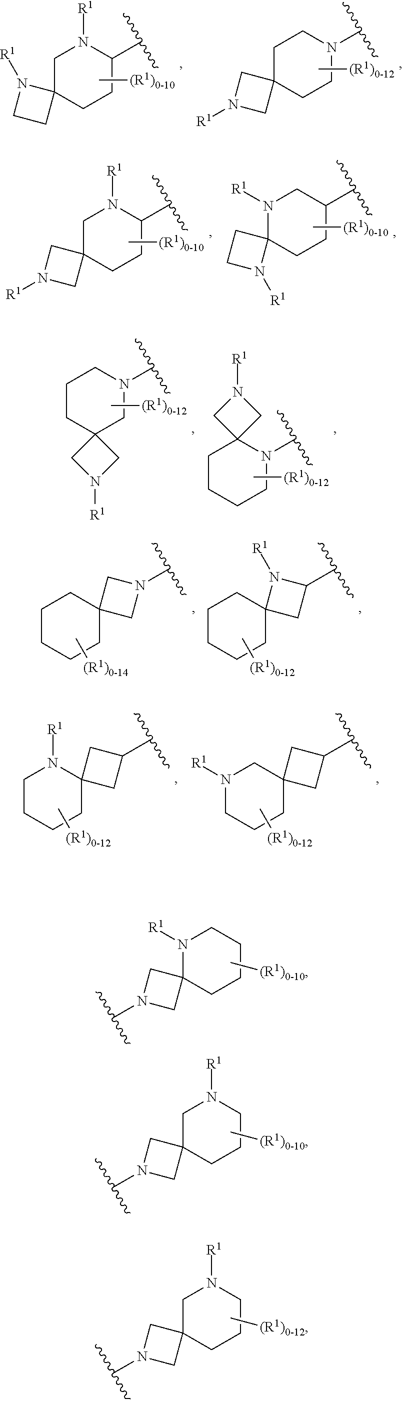

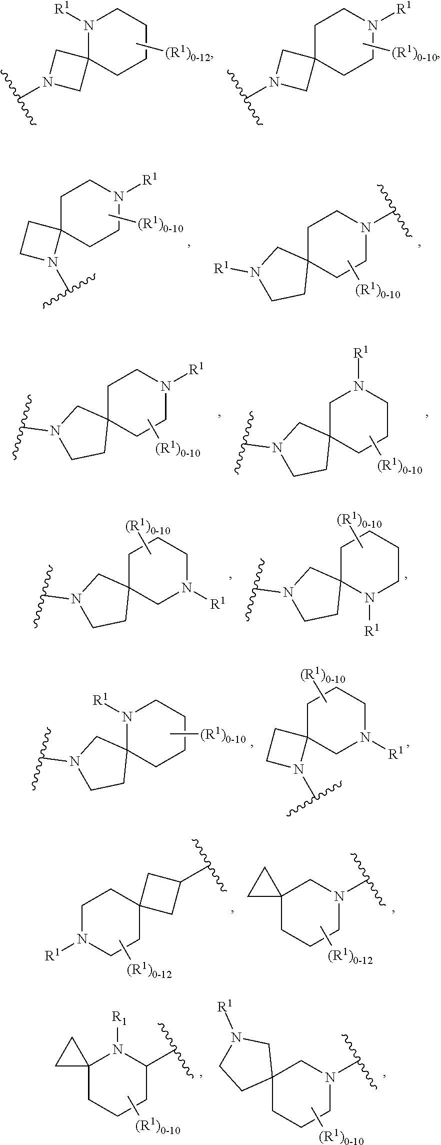

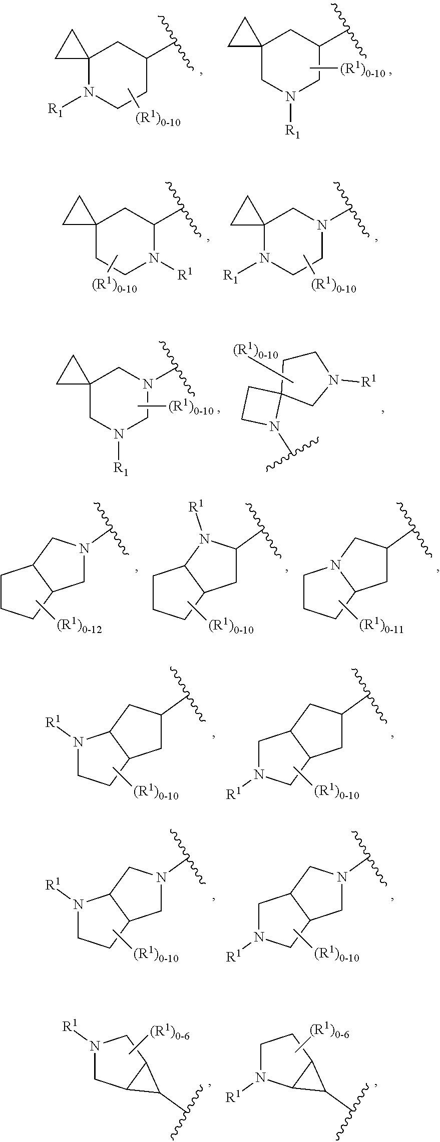

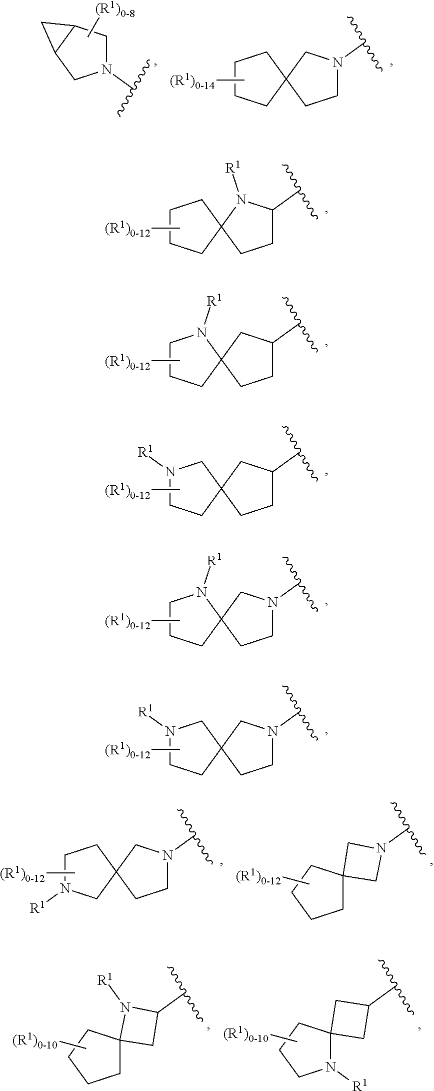

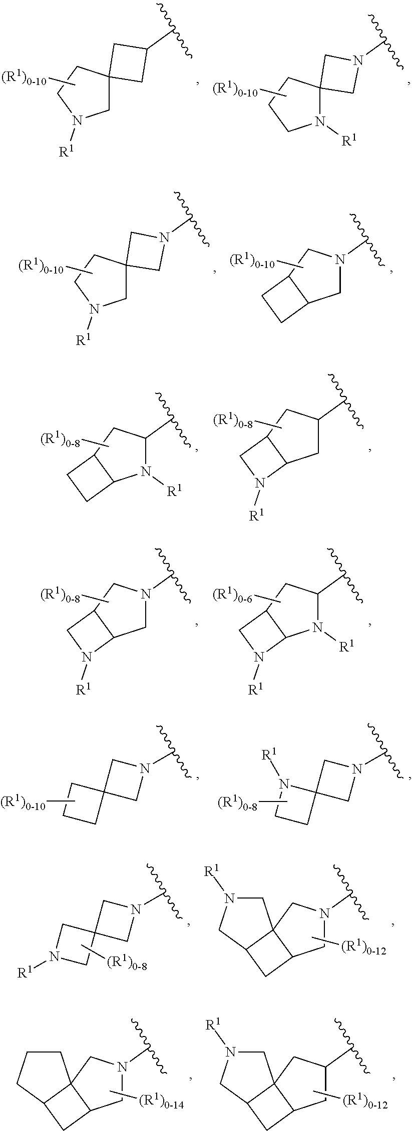

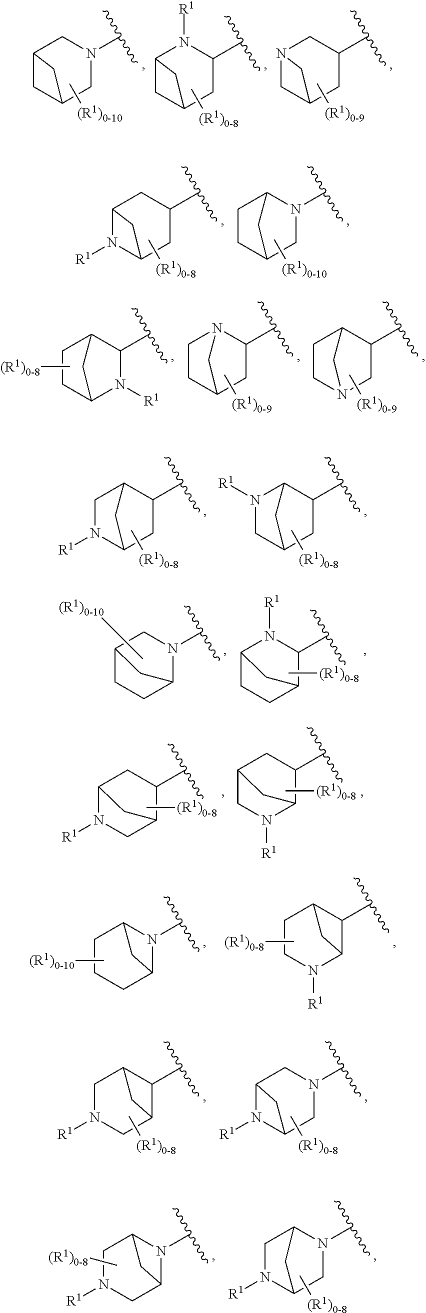

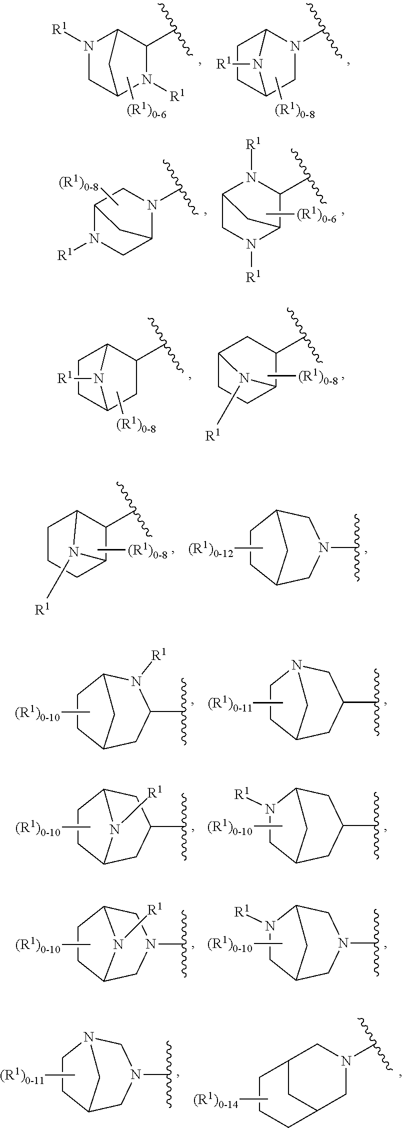

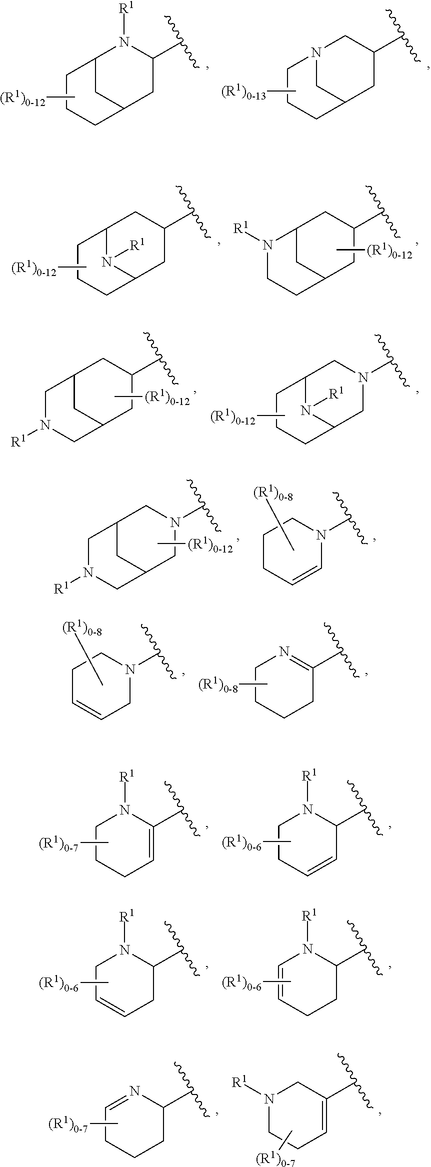

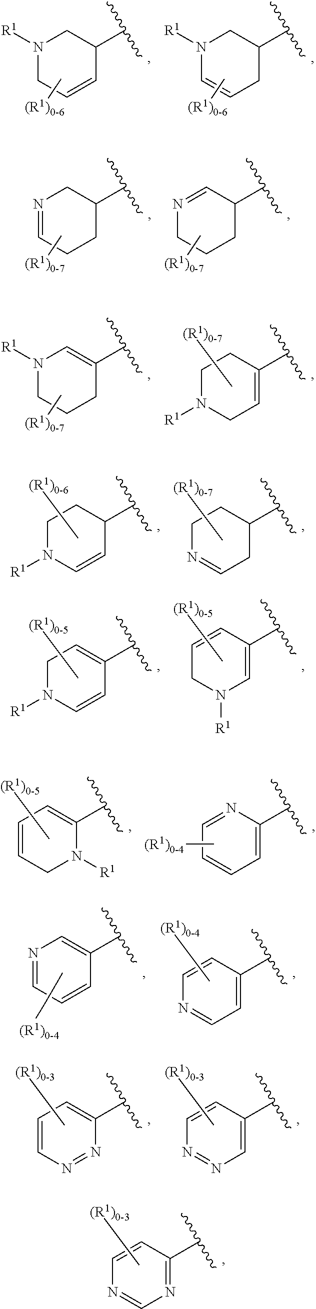

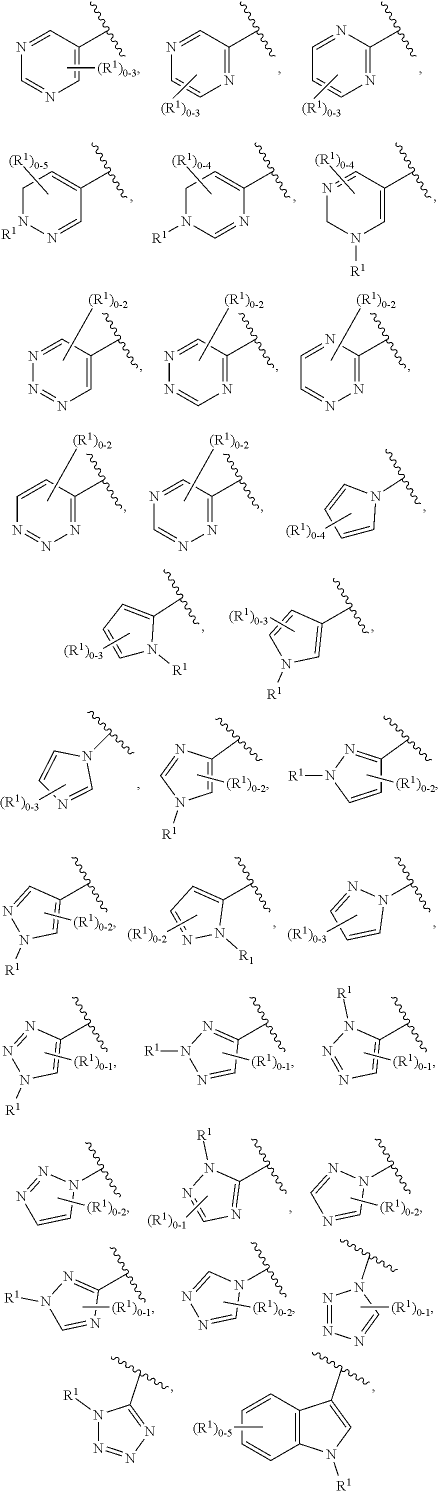

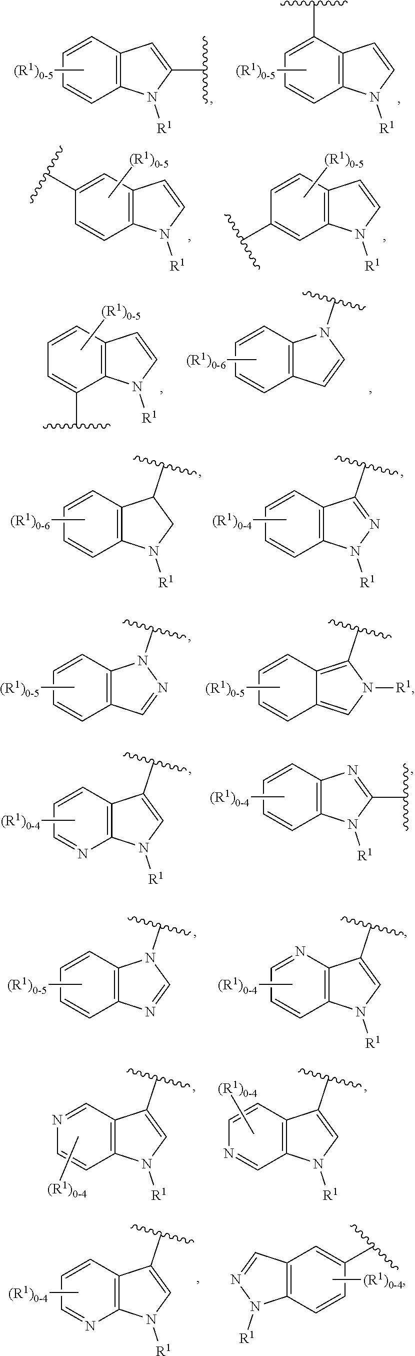

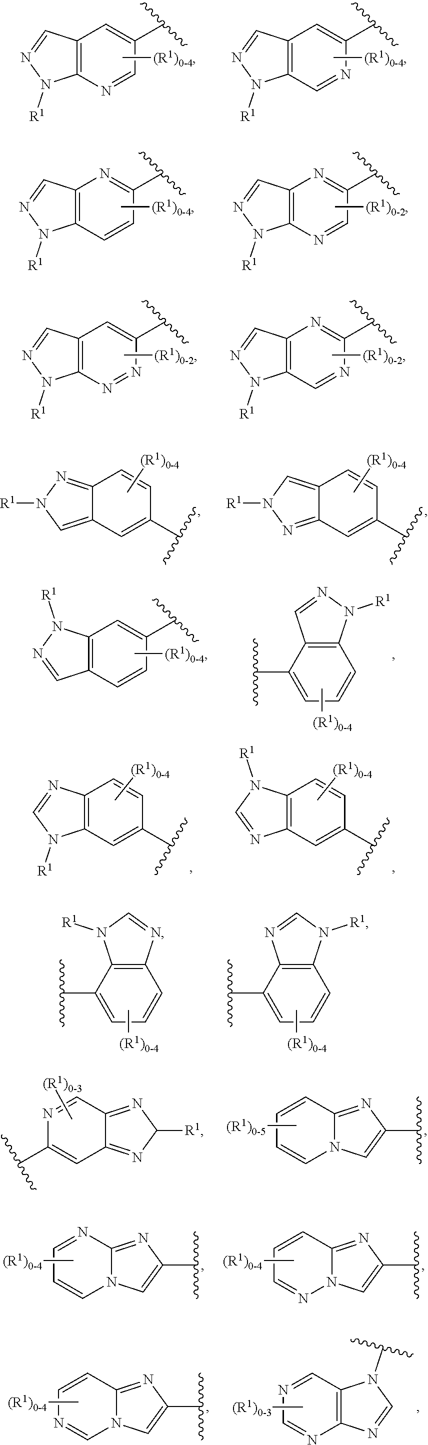

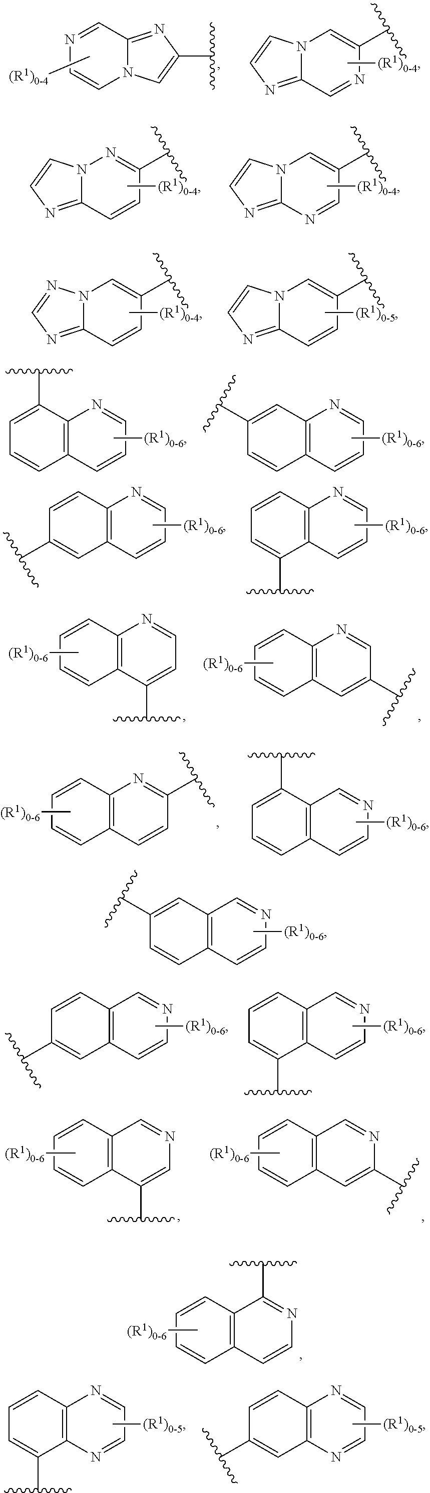

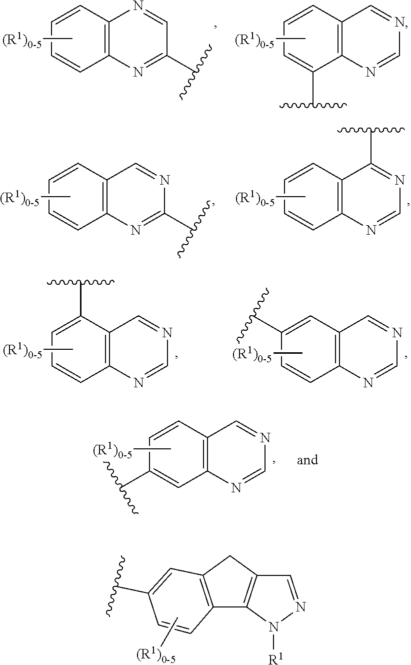

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, each of A and B are independently selected from:

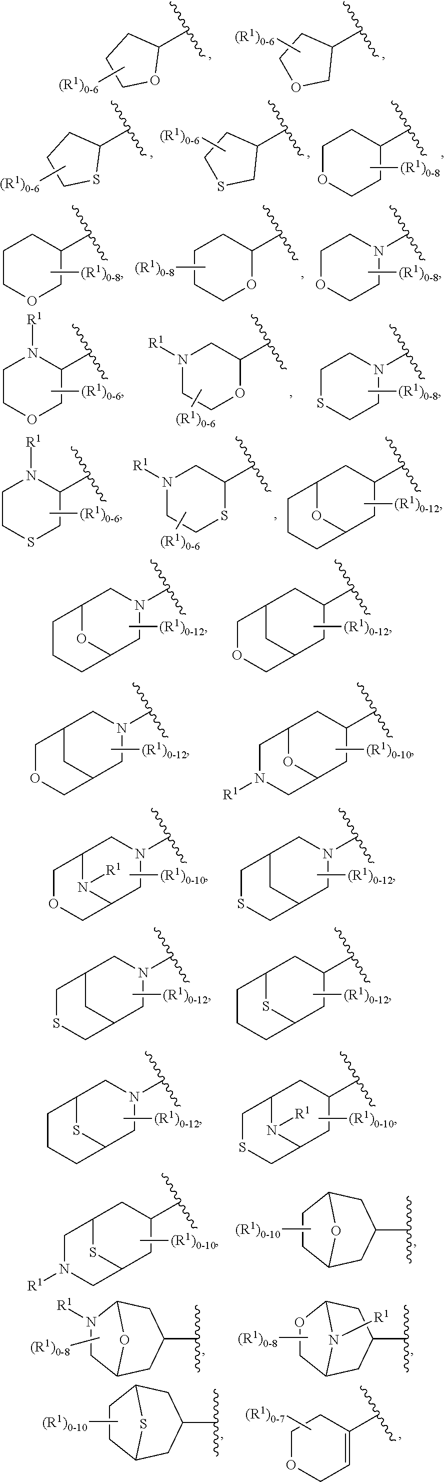

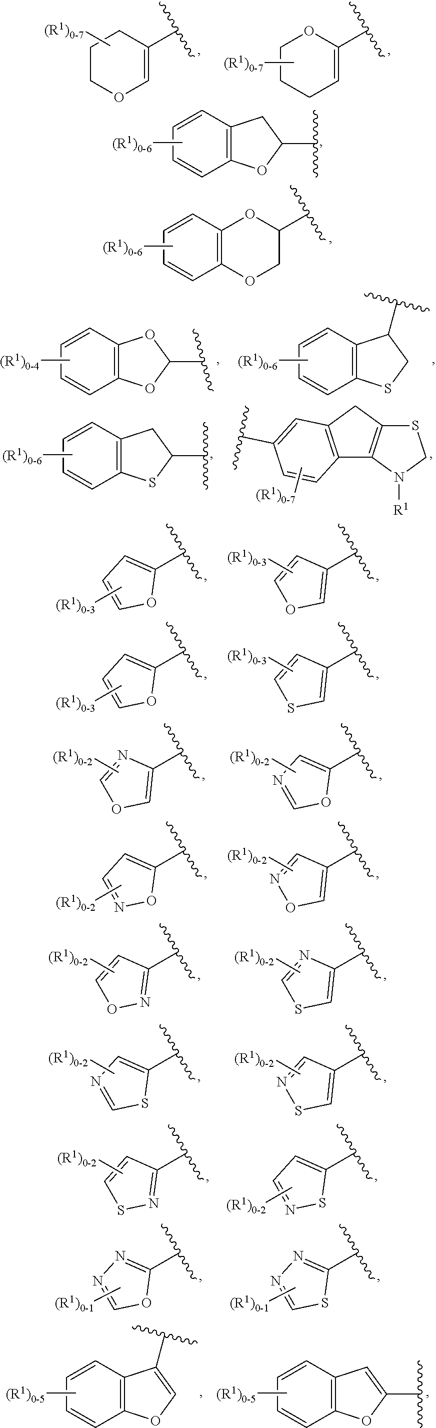

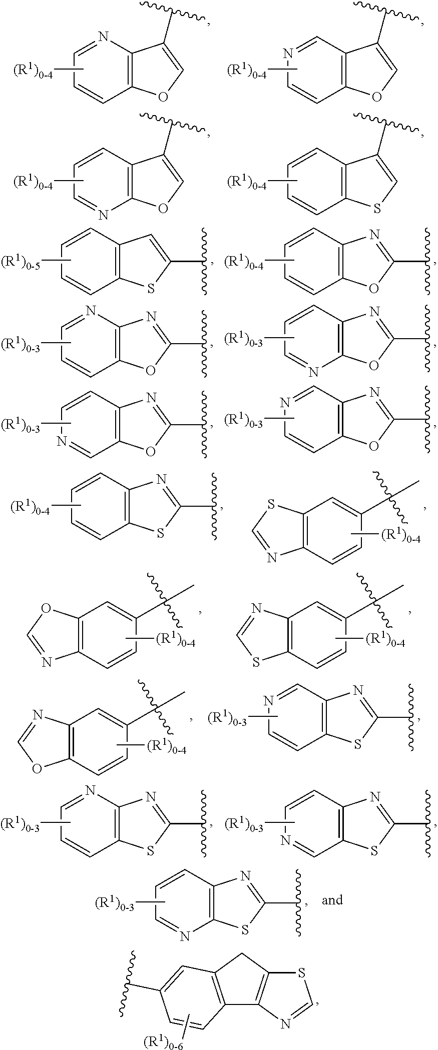

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, one of A and B is independently selected from

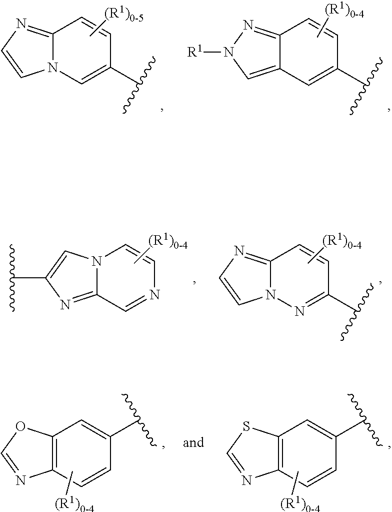

and, wherein R¹ is as described herein. In some embodiments, one of A and B is independently selected from

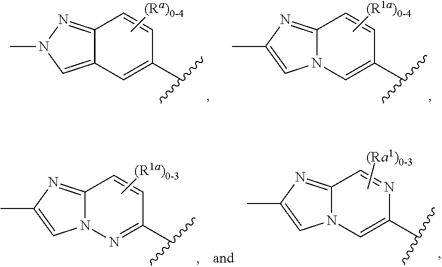

wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷. In some embodiments, one of A and B is independently

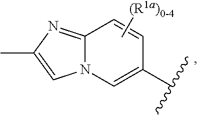

wherein each R^{1a} is independently C₁-C₆-alkyl, C₁-C₆-heteroalkyl, C₁-C₆-haloalkyl, halo, cyano, or OR^{A}, and each alkyl, heteroalkyl, and haloalkyl is optionally substituted with one or more R⁷.

In some embodiments, one of A and B is independently selected from

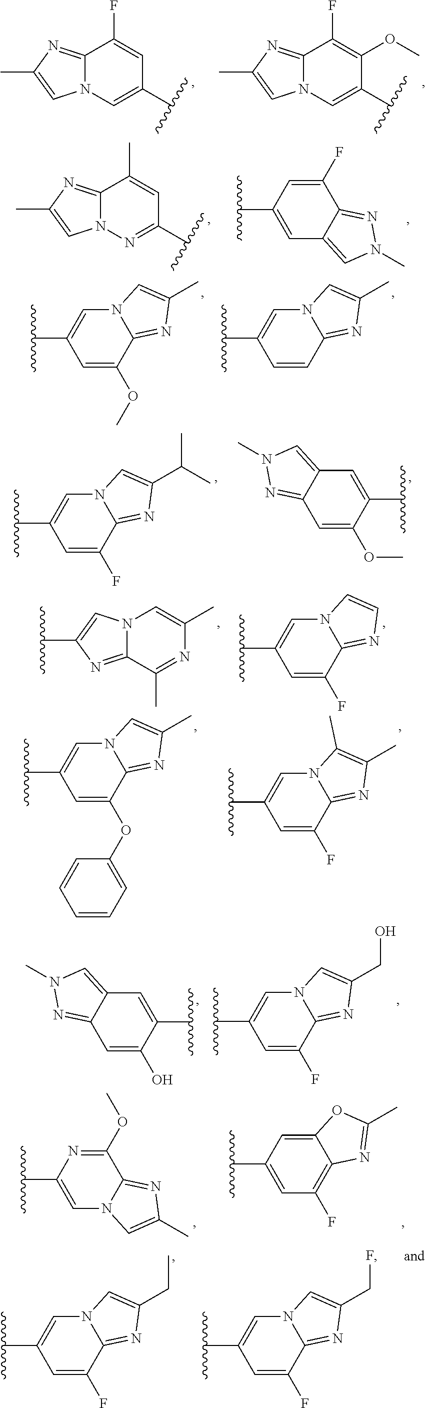

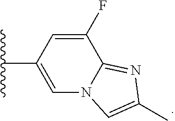 [SMILES: *c1cc(F)c2nc(C)cn2c1]

In some embodiments, one of A and B is independently selected from

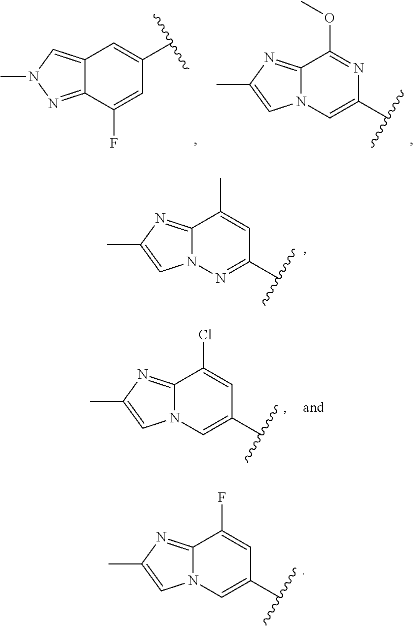 [SMILES: *c1cc(F)c2nn(C)cc2c1 ; *c1cn2cc(C)nc2c(OC)n1 ; *c1cc(C)c2nc(C)cn2n1 ; *c1cc(Cl)c2nc(C)cn2c1 ; *c1cc(F)c2nc(C)cn2c1]

In some embodiments, one of A and B is independently

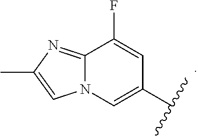 [SMILES: *c1cc(F)c2nc(C)cn2c1]

In some embodiments, one of A and B is independently a monocyclic heterocyclyl or bicyclic heterocyclyl, each of which is optionally substituted with one or more R¹. In some embodiments, one of A and B is independently a nitrogen-containing heterocyclyl optionally substituted with one or more R¹. In some embodiments, one of A and B is independently a 4-8 membered heterocyclyl optionally substituted with one or more R¹. In some embodiments, one of A and B is independently selected from

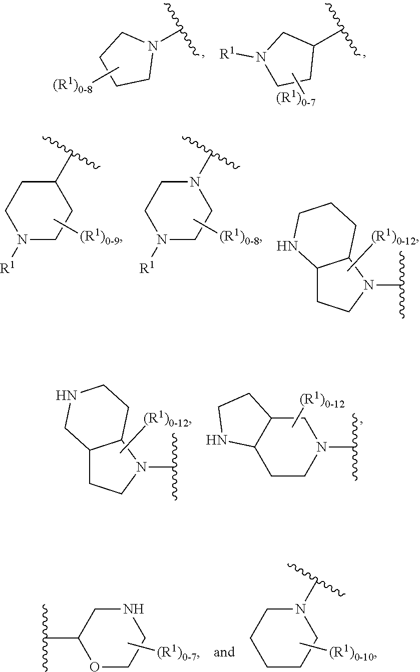

wherein R¹ is as described herein. In some embodiments, one of A and B is independently selected from

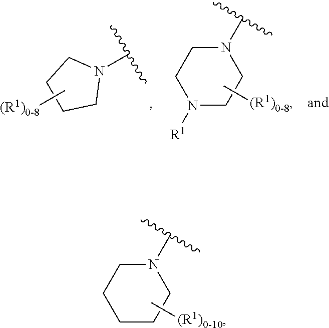

wherein R¹ is as described herein. In some embodiments, one of A and B is

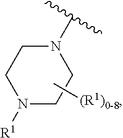

wherein R¹ is as described herein. In some embodiments, A is selected from

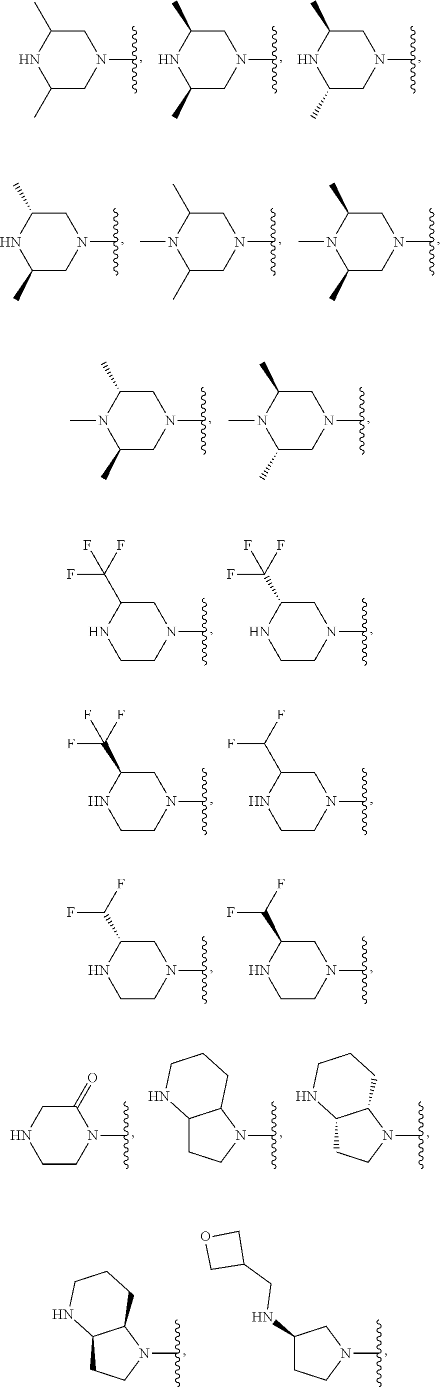

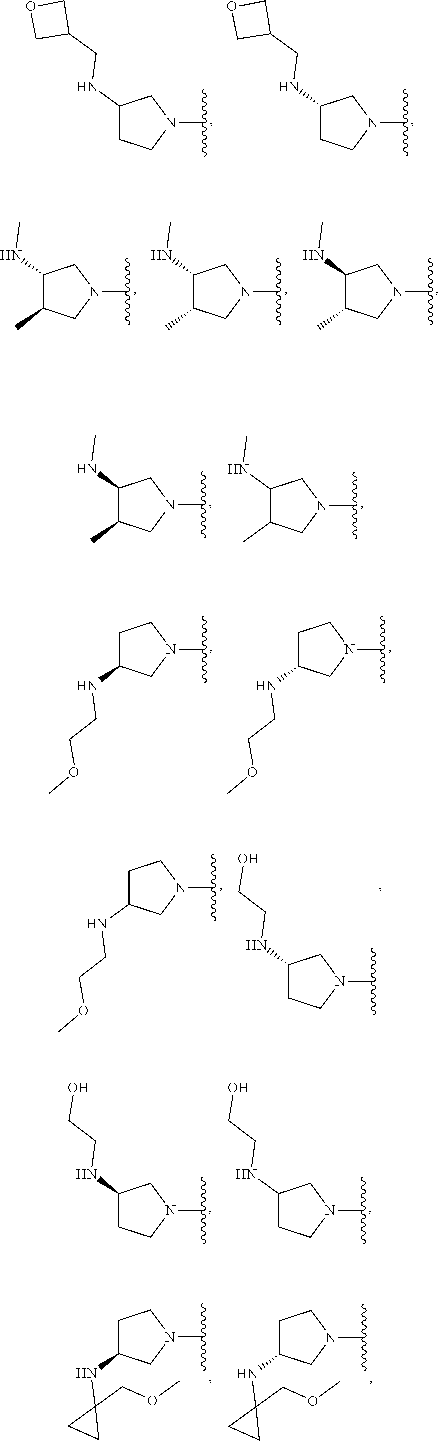

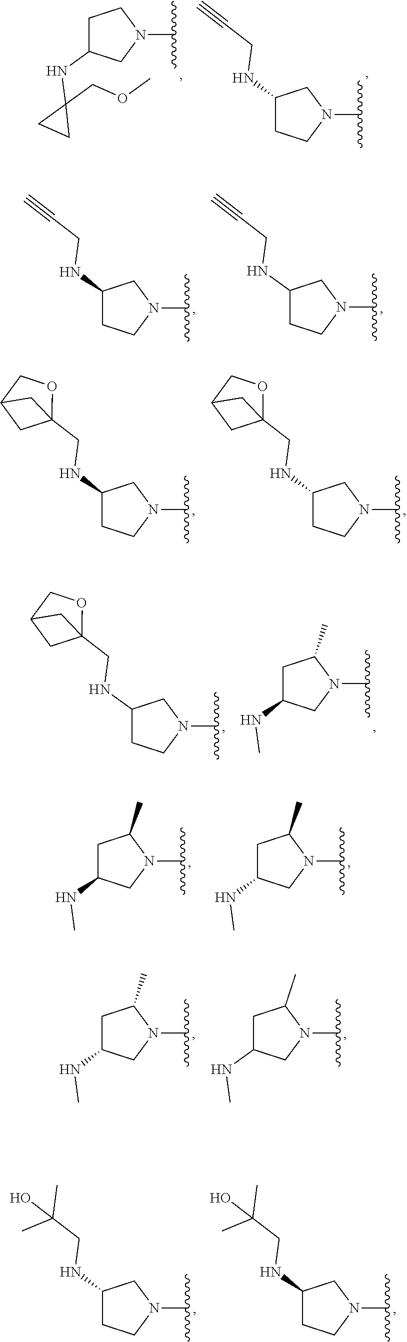

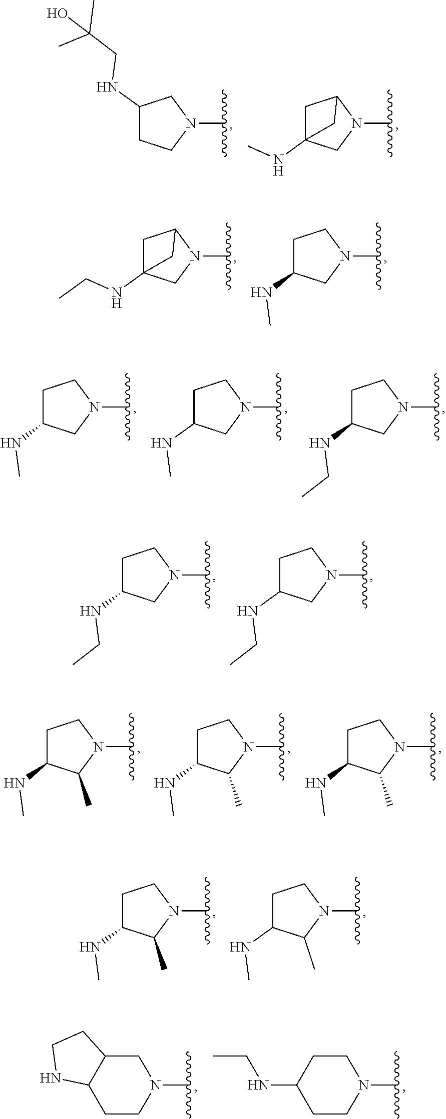

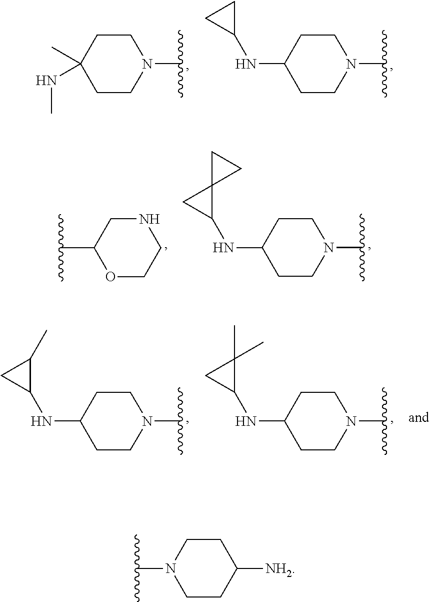

As generally described herein, for Formulas (I) and (II), L¹ and L² each independently may be absent or refer to a C₁-C₆-alkylene, C₁-C₆-heteroalkylene, —O—, —C(O)—, —N(R⁴)—, —N(R⁴)C(O)—, or —C(O)N(R⁴)— group, wherein each alkylene and heteroalkylene is optionally substituted with one or more R⁵.

As generally described herein, for Formula (I), X, Y, and Z each independently refer to C(R^{3a}), C(R^{3a})(R^{3b}), N, or N(R^{3c}), or O. In some embodiments, at least one of X, Y, and Z is either N or N(R^{3c}). In some embodiments, at least one of X, Y, and Z is O. In some embodiments, at least two of X, Y, and Z is N or N(R^{3c}). In some embodiments, X is N. In some embodiments, X is N(R^{3c}). In some embodiments, X is O. In some embodiments, X is C(R^{3a}) (e.g., CH). In some embodiments, X is C(R^{3a})(R^{3b}). In some embodiments, Y is N. In some embodiments, Y is N(R^{3c}). In some embodiments, Y is C(R^{3a}) (e.g., CH). In some embodiments, Y is C(R^{3a}) C(R^{3b}). In some embodiments, Z is N. In some embodiments, Z is N(R^{3c}). In some embodiments, Z is C(R^{3a}) (e.g., CH). In some embodiments, Z is C(R^{3a}) C(R^{3b}). In some embodiments, two of X, Y, and Z are N, and the other of X, Y, and Z is C(R^{3a}) (e.g., CH). In some embodiments, one of X, Y, and Z is C(R^{3a}) (e.g., CH), and the others of X, Y, and Z are each independently N. In some embodiments, X and Y are each independently N, and Z is C(R^{3a}) (e.g., CH). In some embodiments, X is C(R^{3a}) (e.g., CH), and Y and Z are each independently N.

In some embodiments, X, Y, and Z are each independently N or C(R^{3a}), wherein at least one of X, Y, and Z is N and the bonds in the ring comprising X, Y, and Z may be single or double bonds as valency permits.

In some embodiments, X is C(R^{3a}), Y is C(R^{3a}), and Z is O. In some embodiments, X is C(R^{3a}), Y is C(R^{3a}), Z is O, and y is 0. In some embodiments, X is C(R^{3a}), Y is C(R^{3a}), Z is O, and the bond between X and Y is a double bond. In some embodiments, X is C(R^{3a}), Y is C(R^{3a}), Z is O, and the bond between Y and Z is a single bond.

In some embodiments for Formulas (I) and (II), R¹ is hydrogen. In some embodiments, R¹ is C₁-C₆-alkyl. In some embodiments, R¹ is C₂-C₆-alkenyl. In some embodiments, R¹ is C₂-C₆-alkynyl. In some embodiments, R¹ is C₁-C₆-heteroalkyl. In some embodiments, R¹ is C₁-C₆-haloalkyl (e.g., —CF₃). In some embodiments, R¹ is C₁-alkyl (e.g., methyl). In some embodiments, R¹ is unsubstituted C₁-C₆-alkyl, unsubstituted C₂-C₆-alkenyl, unsubstituted C₂-C₆-alkynyl, unsubstituted C₁-C₆-heteroalkyl, or unsubstituted C₁-C₆-haloalkyl. In some embodiments, R¹ is C₁-C₆-alkyl substituted with one or more R⁶. In some embodiments, R¹ is C₂-C₆-alkenyl substituted with one or more R⁶. In some embodiments, R¹ is C₂-C₆-alkynyl substituted with one or more R⁶. In some embodiments, R¹ is C₁-C₆-heteroalkyl substituted with one or more R⁶. In some embodiments, R¹ is C₁-C₆-haloalkyl substituted with one or more R⁶. In some embodiments, R¹ is methyl.

In some embodiments, R¹ is cycloalkyl (e.g., 3-7 membered cycloalkyl). In some embodiments, R¹ is heterocyclyl (e.g., 3-7 membered heterocyclyl). In some embodiments, R¹ is aryl. In some embodiments, R¹ is C₁-C₆ alkylene-aryl (e.g., benzyl). In some embodiments, R¹ is C₁-C₆ alkenylene-aryl. In some embodiments, R¹ is C₁-C₆ alkylene-heteroaryl. In some embodiments, R¹ is heteroaryl. In some embodiments, R¹ is unsubstituted cycloalkyl, unsubstituted heterocyclyl, unsubstituted aryl, unsubstituted C₁-C₆ alkylene-aryl, unsubstituted C₁-C₆ alkenylene-aryl, unsubstituted C₁-C₆ alkylene-heteroaryl, or unsubstituted heteroaryl. In some embodiments, R¹ is cycloalkyl substituted with one or more R⁶. In some embodiments, R¹ is heterocyclyl substituted with one or more R⁶. In some embodiments, R¹ is aryl substituted with one or more R⁶. In some embodiments, R¹ is C₁-C₆ alkylene-aryl substituted with one or more R⁶. In some embodiments, R¹ is C₁-C₆ alkenylene-aryl substituted with one or more R⁶. In some embodiments, R¹ is C₁-C₆ alkylene-heteroaryl substituted with one or more R⁶. In some embodiments, R¹ is heteroaryl substituted with one or more R⁶.

In some embodiments, R¹ is —OR^{A}. In some embodiments, R¹ is —NR^{B}R^{C} (e.g., NH₂ or NMe₂). In some embodiments, R¹ is —NR^{B}C(O)R^{D}. In some embodiments, R¹ is —C(O)NR^{B}R^{C}. In some embodiments, R¹ is —C(O)R^{D}. In some embodiments, R¹ is —C(O)OR^{D}. In some embodiments, R¹ is —SR^{E}. In some embodiments, R¹ is —S(O)ₓR^{D}. In some embodiments, R¹ is halo, e.g., fluoro, chloro, bromo, or iodo. In some embodiments, R¹ is cyano. In some embodiments, R¹ is nitro (—NO₂). In some embodiments, R¹ is oxo.

In some embodiments, two R¹ groups, together with the atoms to which they are attached, form a 3-7-membered cycloalkyl. In some embodiments, two R¹ groups, together with the atoms to which they are attached, form a 3-7-membered heterocyclyl. In some embodiments, two R¹ groups, together with the atoms to which they are attached, form a 5- or 6-membered aryl. In some embodiments, two R¹ groups, together with the atoms to which they are attached, form a 5- or 6-membered heteroaryl. The cycloalkyl, heterocyclyl, aryl, or heteroaryl may be substituted with one or more R⁶.

In some embodiments for Formulas (I) and (II), R² is hydrogen. In some embodiments, R² is halo (e.g., fluoro, chloro, bromo, or iodo). In some embodiments, R² is cyano. In some embodiments, R² is C₁-C₆-alkyl. In some embodiments, R² is C₂-C₆-alkenyl. In some embodiments, R² is C₂-C₆-alkynyl. In some embodiments, R² is —OR^{A} (e.g., —OH).

In some embodiments, R^{3a}, R^{3b}, or both are independently hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}. In some embodiments, R^{3a} and R^{3b} are each independently hydrogen or C₁-C₆-alkyl. In some embodiments, R^{3a} is hydrogen. In some embodiments, R^{3b} is hydrogen. In some embodiments, R^{3a} is C₁-C₆-alkyl (e.g., methyl). In some embodiments, R^{3b} is C₁-C₆-alkyl (e.g., methyl). In some embodiments, R^{3a} is halo (e.g., fluoro, chloro, bromo, or iodo). In some embodiments, R^{3b} is halo (e.g., fluoro, chloro, bromo, or iodo). In some embodiments, R^{3a} is cyano. In some embodiments, R^{3b} is cyano. In some embodiments, R^{3a} is —OR^{A} (e.g., —OH). In some embodiments, R^{3b} is —OR^{A} (e.g., —OH). In some embodiments, R^{3a} is —NR^{B}R^{C}. In some embodiments, R^{3b} is —NR^{B}R^{C}. In some embodiments, R^{3a} is —C(O)R^{D}. In some embodiments, R^{3b} is —C(O)R^{D}. In some embodiments, R^{3a} is —C(O)OR^{D}. In some embodiments, R^{3b} is —C(O)OR^{D}. In some embodiments, each of R^{3a} and R^{3b}, together with the carbon atom to which they are attached, form an oxo group.

In some embodiments, R^{3c} is hydrogen. In some embodiments, R^{3c} is C₁-C₆-alkyl. In some embodiments, R^{3c} is methyl. In some embodiments, R^{3c} is not hydrogen. In some embodiments, R^{3c} is not methyl. In some embodiments, R^{3c} is C₁-C₆ alkyl. In some embodiments, R^{3c} is C₁-C₆ substituted with one or more R⁸.

In some embodiments, R⁴ is hydrogen. In some embodiments, R⁴ is C₁-C₆ alkyl. In some embodiments, R⁴ is C₁-C₆ haloalkyl (e.g., —CF₃ or —CHF₂). In some embodiments, R⁴ is methyl.

In some embodiments, R⁵ is hydrogen. In some embodiments, R⁵ is C₁-C₆-alkyl. In some embodiments, R⁵ is C₁-C₆-heteroalkyl. In some embodiments, R⁵ is C₁-C₆-haloalkyl. In some embodiments, R⁵ is cycloalkyl. In some embodiments, R⁵ is halo (e.g., fluoro, chloro, bromo, or iodo). In some embodiments, R⁵ is cyano. In some embodiments, R⁵ is oxo. In some embodiments, R⁵ is —OR^{A}. In some embodiments, R⁵ is —NR^{B}R^{C}. In some embodiments, R⁵ is —C(O)R^{D} or —C(O)OR^{D}.

In some embodiments, R⁶ is C₁-C₆-alkyl. In some embodiments, R⁶ is C₂-C₆-alkenyl. In some embodiments, R⁶ is C₂-C₆-alkynyl. In some embodiments, R⁶ is C₁-C₆-heteroalkyl. In some embodiments, R⁶ is C₁-C₆-haloalkyl. In some embodiments, R⁶ is unsubstituted C₁-C₆-alkyl, unsubstituted C₂-C₆-alkenyl, unsubstituted C₂-C₆-alkynyl, unsubstituted C₁-C₆-haloalkyl, or unsubstituted C₁-C₆-heteroalkyl. In some embodiments, R⁶ is C₁-C₆-alkyl substituted with one or more R¹¹. In some embodiments, R⁶ is C₂-C₆-alkenyl substituted with one or more R¹¹. In some embodiments, R⁶ is C₂-C₆-alkynyl substituted with one or more R¹¹. In some embodiments, R⁶ is C₁-C₆-haloalkyl substituted with one or more R¹¹. In some embodiments, R⁶ is C₁-C₆-heteroalkyl substituted with one or more R¹¹.

In some embodiments, R⁶ is cycloalkyl. In some embodiments, R⁶ is heterocyclyl. In some embodiments, R⁶ is aryl. In some embodiments, R⁶ is heteroaryl. In some embodiments, R⁶ is unsubstituted cycloalkyl, unsubstituted heterocyclyl, unsubstituted aryl, or unsubstituted heteroaryl. In some embodiments, R⁶ is cycloalkyl substituted with one or more R¹¹. In some embodiments, R⁶ is heterocyclyl substituted with one or more R¹¹. In some embodiments, R⁶ is aryl substituted with one or more R¹¹. In some embodiments, R⁶ is heteroaryl substituted with one or more R¹¹.

In some embodiments, R⁶ is halo (e.g., fluoro, chloro, bromo, or iodo). In some embodiments, R⁶ is cyano. In some embodiments, R⁶ is oxo. In some embodiments, R⁶ is —OR^{A}. In some embodiments, R⁶ is —NR^{B}R^{C}. In some embodiments, R⁶ is —NR^{B}C(O)R^{D}. In some embodiments, R⁶ is —NO₂. In some embodiments, R⁶ is —C(O)NR^{B}R^{C}. In some embodiments, R⁶ is —C(O)R^{D}. In some embodiments, R⁶ is —C(O)OR^{D}. In some embodiments, R⁶ is —SR^{E}. In some embodiments, R⁶ is —S(O)ₓR^{D}.

In some embodiments, R⁷ is C₁-C₆-alkyl. In some embodiments, R⁷ is halo (e.g., fluoro, chloro, bromo, or iodo). In some embodiments, R⁷ is cyano. In some embodiments, R⁷ is oxo. In some embodiments, R⁷ is —OR^{A1} (e.g., —OH).

In some embodiments, R¹¹ is C₁-C₆-alkyl. In some embodiments, R¹¹ is C₁-C₆-heteroalkyl. In some embodiments, R¹¹ is C₁-C₆-haloalkyl (e.g., —CF₃). In some embodiments, R¹¹ is cycloalkyl. In some embodiments, R¹¹ is heterocyclyl. In some embodiments, R¹¹ is aryl. In some embodiments, R¹¹ is heteroaryl. In some embodiments, R¹¹ is halo. In some embodiments, R¹¹ is cyano. In some embodiments, R¹¹ is oxo. In some embodiments, R¹¹ is —OR^{A}.

In some embodiments for Formulas (I) and (II), R^{A} is hydrogen. In some embodiments, R^{A} is C₁-C₆ alkyl (e.g., methyl). In some embodiments, R^{A} is C₁-C₆ haloalkyl. In some embodiments, R^{A} is aryl. In some embodiments, R^{A} is heteroaryl. In some embodiments, R^{A} is C₁-C₆ alkylene-aryl (e.g., benzyl). In some embodiments, R^{A} is C₁-C₆ alkylene-heteroaryl. In some embodiments, R^{A} is C(O)R^{D}. In some embodiments, R^{A} is —S(O)ₓR^{D}.

In some embodiments, R^{B}, R^{C}, or both are independently hydrogen, C₁-C₆-alkyl, C₁-C₆-heteroalkyl, cycloalkyl, heterocyclyl, or —OR^{A}. In some embodiments, each of R^{B} and R^{C} is independently hydrogen. In some embodiments, each of R^{B} and R^{C} is independently C₁-C₆ alkyl. In some embodiments, one of R^{B} and R^{C} is hydrogen, and the other of R^{B} and R^{C} is C₁-C₆ alkyl. In some embodiments, R^{B} and R^{C} together with the atom to which they are attached form a 3-7-membered heterocyclyl ring optionally substituted with one or more of R⁷.

In some embodiments, R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl (e.g., benzyl), or C₁-C₆ alkylene-heteroaryl. In some embodiments, R^{D} is hydrogen. In some embodiments, R^{D} is C₁-C₆ alkyl (e.g., methyl). In some embodiments, R^{D} is C₁-C₆ heteroalkyl. In some embodiments, R^{D} is C₁-C₆ haloalkyl. In some embodiments, R^{D} is cycloalkyl. In some embodiments, R^{D} is heterocyclyl. In some embodiments, R^{D} is aryl. In some embodiments, R^{D} is heteroaryl. In some embodiments, R^{D} is C₁-C₆ alkylene-aryl (e.g., benzyl). In some embodiments, R^{D} is C₁-C₆ alkylene-heteroaryl.

In some embodiments, R^{A1} is hydrogen. In some embodiments, R^{A1} is C₁-C₆-alkyl (e.g., methyl).

In some embodiments, m is 0, 1, or 2. In some embodiments, m is 0. In some embodiments, m is 1. In some embodiments, m is 2. In some embodiments, x is 0, 1, or 2. In some embodiments, x is 0. In some embodiments, x is 1. In some embodiments, x is 2. In some embodiments y is 0 or 1. In some embodiments, y is 0.

In some embodiments, the compound of Formula (I) is a compound of Formula (I-a):

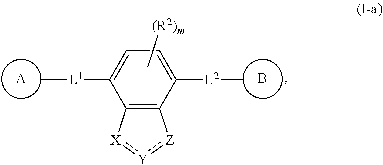

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; X, Y, and Z are each independently C(R^{3a}), C(R^{3a})(R^{3b}), N, N(R^{3c}), S, or O, wherein at least one of X, Y, and Z is N, N(R^{3c}), or O, and the bonds in the ring comprising X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} and R^{3b} are each independently hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}; or each of R^{3a} and R^{3b} together with the carbon atom to which they are attached, form an oxo group; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}, each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} and R^{E} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-b):

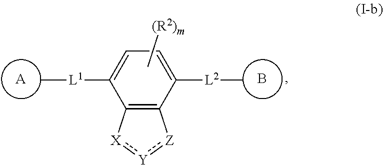

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; X, Y, and Z are each independently C(R^{3a}), C(R^{3a})(R^{3b}), N, N(R^{3c}), or O, wherein at least one of X, Y, and Z is N, N(R^{3c}), or O, and the bonds in the ring comprising X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} and R^{3b} are each independently hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}; or each of R^{3a} and R^{3b}, together with the carbon atom to which they are attached, form an oxo group; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the with one or more R⁷; each R^{D} and R^{E} is independently hydrogen, C₁-C₆ alkyl, C₂-C₆ alkenyl, C₂—C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-c):

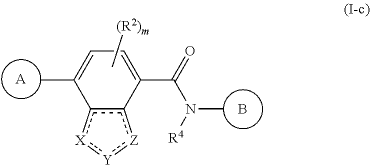

or a pharmaceutically acceptable salt, solvate, hydrate, tautomer, or stereoisomer thereof, wherein A is a nitrogen-containing heterocyclyl optionally substituted with one or more R¹; B is a nitrogen-containing heteroaryl and optionally substituted with one or more R¹; X, Y, and Z are each independently C(R^{3a}), C(R^{3a})(R^{3b}), N, N(R^{3c}), S, or O, wherein at least one of X, Y, and Z is N, N(R^{3c}), or O, and the bonds in the ring comprising X, Y, and Z may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ heteroalkylene-aryl, heteroaryl, C₁-C₆ alkylene-heteroaryl, C₁-C₆ heteroalkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; R^{3a} and R^{3b} are each independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; or each of R^{3a} and R^{3b}, together with the carbon atom to which they are attached, form an oxo group; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸; each R⁴ is independently hydrogen, C₁-C₆-alkyl, C₁-C₆-heteroalkyl, or C₁-C₆-haloalkyl; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹²; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R¹² is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₂-C₆ alkenyl, C₂-C₆ alkynyl, C₁-C₆ haloalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, —C(O)R^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-d):

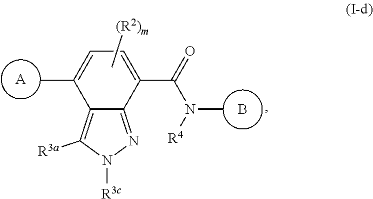

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} and R^{3b} are each independently hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}; or each of R^{3a} and R^{3b}, together with the carbon atom to which they are attached, form an oxo group; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸, each R⁴ is independently hydrogen, C₁-C₆-alkyl, C₁-C₆-heteroalkyl, or C₁-C₆-haloalkyl; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the with one or more R⁷; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-e):

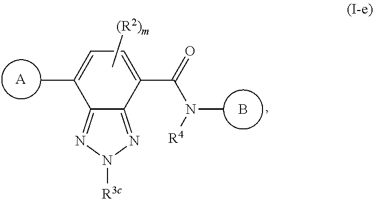

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}, R^{3a} and R^{3b} are each independently hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}; or each of R^{3a} and R^{3b}, together with the carbon atom to which they are attached, form an oxo group; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D} wherein each alkyl, alkylene, alkenyl, alkynyl, heteroalkyl, haloalkyl, cycloalkyl, heterocyclyl, aryl, and heteroaryl is optionally substituted with one or more R⁸; each R⁴ is independently hydrogen, C₁-C₆-alkyl, C₁-C₆-heteroalkyl, or C₁-C₆-haloalkyl; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-f):

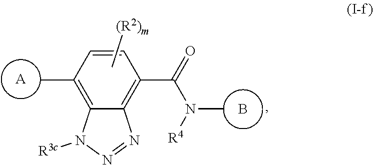

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} and R^{3b} are each independently hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}; or each of R^{3a} and R^{3b}, together with the carbon atom to which they are attached, form an oxo group; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸; each R⁴ is independently hydrogen, C₁-C₆-alkyl, C₁-C₆-heteroalkyl, or C₁-C₆-haloalkyl; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-g):

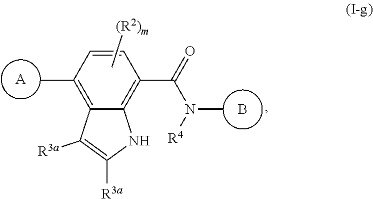

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} and R^{3b} are each independently hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}; or each of R^{3a} and R^{3b}, together with the carbon atom to which they are attached, form an oxo group; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸, each R⁴ is independently hydrogen, C₁-C₆-alkyl, C₁-C₆-heteroalkyl, or C₁-C₆-haloalkyl; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}, each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-h):

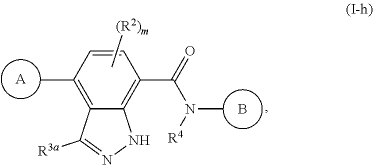

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}, R^{3a} and R^{3b} are each independently hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}; or each of R^{3a} and R^{3b}, together with the carbon atom to which they are attached, form an oxo group; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D} wherein each alkyl, alkylene, alkenyl, alkynyl, heteroalkyl, haloalkyl, cycloalkyl, heterocyclyl, aryl, and heteroaryl is optionally substituted with one or more R⁸; each R⁴ is independently hydrogen, C₁-C₆-alkyl, C₁-C₆-heteroalkyl, or C₁-C₆-haloalkyl; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}, or R^{B} and R^{C} together with the atom to which they are attached form a 3-7-membered heterocyclyl ring optionally substituted with one or more R⁷; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-i):

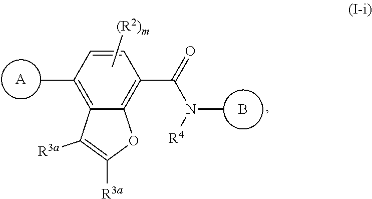

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} and R^{3b} are each independently hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}; or each of R^{3a} and R^{3b}, together with the carbon atom to which they are attached, form an oxo group; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸, each R⁴ is independently hydrogen, C₁-C₆-alkyl, C₁-C₆-heteroalkyl, or C₁-C₆-haloalkyl; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the with one or more R⁷; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-j):

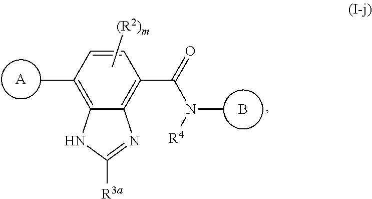

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}, R^{3a} and R^{3b} are each independently hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}; or each of R^{3a} and R^{3b}, together with the carbon atom to which they are attached, form an oxo group; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D} wherein each alkyl, alkylene, alkenyl, alkynyl, heteroalkyl, haloalkyl, cycloalkyl, heterocyclyl, aryl, and heteroaryl is optionally substituted with one or more R⁸, each R⁴ is independently hydrogen, C₁-C₆-alkyl, C₁-C₆-heteroalkyl, or C₁-C₆-haloalkyl; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-1):

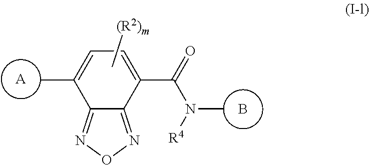

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} and R^{3b} are each independently hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}; or each of R^{3a} and R^{3b}, together with the carbon atom to which they are attached, form an oxo group; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸; each R⁴ is independently hydrogen, C₁-C₆-alkyl, C₁-C₆-heteroalkyl, or C₁-C₆-haloalkyl; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2.

In some embodiments, the compound of Formula (I) is a compound of Formula (I-m):

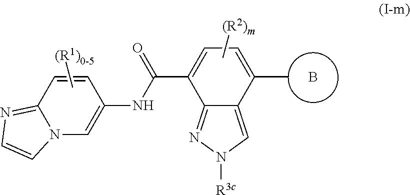

or a pharmaceutically acceptable salt, solvate, hydrate, tautomer, or stereoisomer thereof, wherein B is cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} and R^{3b} are each independently hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}; or each of R^{3a} and R^{3b}, together with the carbon atom to which they are attached, form an oxo group; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸; each R⁴ is independently hydrogen, C₁-C₆-alkyl, C₁-C₆-heteroalkyl, or C₁-C₆-haloalkyl; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R¹, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁷; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2. In some embodiments, the compound of Formula (I) is selected from a compound in Table 1, or a pharmaceutically acceptable salt, solvate, hydrate, tautomer, or stereoisomer thereof.

In some embodiments, the compound of Formula (I) is a compound of Formula (I-n):

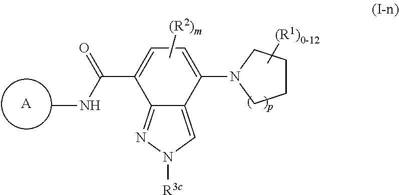

or a pharmaceutically acceptable salt, solvate, hydrate, tautomer, or stereoisomer thereof, wherein A is cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}, R^{3a} and R^{3b} are each independently hydrogen, C₁-C₆-alkyl, C₁-C₆-heteroalkyl, C₁-C₆-haloalkyl, halo, cyano, —OR^{A}, —NR^{B}R^{C}, —C(O)R^{D}, or —C(O)OR^{D}; or each of R^{3a} and R^{3b} together with the carbon atom to which they are attached, form an oxo group; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R¹, wherein each alkyl, alkylene, alkenyl, alkynyl, heteroalkyl, haloalkyl, cycloalkyl, heterocyclyl, aryl, and heteroaryl is optionally substituted with one or more R⁸; each R⁴ is independently hydrogen, C₁-C₆-alkyl, C₁-C₆-heteroalkyl, or C₁-C₆-haloalkyl; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R⁷ is C₁-C₆-alkyl, halo, cyano, oxo, or —OR^{A1}; each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}, or R^{B} and R^{C} together with the atom to which they are attached form a 3-7-membered heterocyclyl ring optionally substituted with one or more R⁷; each R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R^{A1} is hydrogen or C₁-C₆-alkyl; m is 0, 1, or 2; and x is 0, 1, or 2. In some embodiments, the compound of Formula (I) is selected from a compound in Table 1, or a pharmaceutically acceptable salt, solvate, hydrate, tautomer, or stereoisomer thereof.

The present disclosure further features compounds of Formula (II). In some embodiments, the present disclosure features a compound of Formula (II-a):

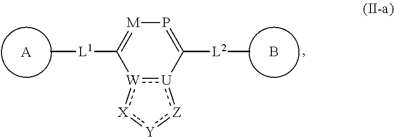

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; M and P are each independently C(R²) or N; U and W are each independently C or N; X, Y, and Z are each independently C(R^{3a}), N, N(R^{3c}), O, or S, wherein at least one of X, Y, and Z is N or N(R^{3c}), and the bonds in the ring comprising U, W, X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}, R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸; each R⁴ is independently hydrogen, C₁-C₆-alkyl, C₁-C₆-heteroalkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}, or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}, each R^{A1} is hydrogen or C₁-C₆-alkyl; and x is 0, 1, or 2.

In some embodiments, the compound of Formula (II) is a compound of Formula (II-b):

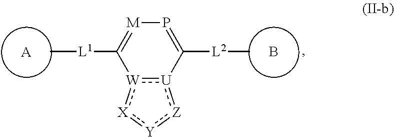

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; M and P are each independently C(R²) or N; U and W are each independently C or N; X, Y, and Z are each independently C(R^{3a}), N, N(R^{3c}) or S, wherein at least one of X, Y, and Z is N or N(R^{3c}), and the bonds in the ring comprising U, W, X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-c):

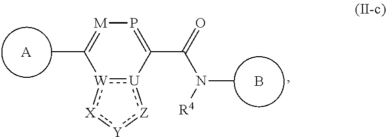

or a pharmaceutically acceptable salt, solvate, hydrate, tautomer, or stereoisomer thereof, wherein A is a nitrogen-containing heterocyclyl optionally substituted with one or more R¹; B is a nitrogen-containing heteroaryl and optionally substituted with one or more R¹; M and P are each independently C(R²) or N; U and W are each independently C or N; X, Y, and Z are each independently C(R^{3a}), C(R^{3a})(R^{3b}), N, N(R^{3c}), S, or O, wherein at least one of X, Y, and Z is N or N(R^{3c}), and the bonds in the ring comprising U, W, X, Y, and Z may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} and R^{3b} are each independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; or each of R^{3a} and R^{3b}, together with the carbon atom to which they are attached, form an oxo group; R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸; each R⁴ is independently hydrogen, C₁-C₆-alkyl, C₁-C₆-heteroalkyl, or C₁-C₆-haloalkyl; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —NR^{B}N(R^{B})(R^{C}), —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}, wherein C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, and C₁-C₆ alkylene-heteroaryl is optionally substituted with one or more R⁷; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}, wherein C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, and C₁-C₆ alkylene-heteroaryl is optionally substituted with one or more R⁷; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-d):

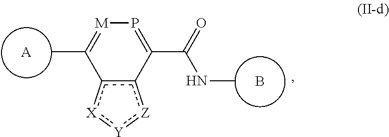

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; M and P are each independently C(R²) or N; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O) \R^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-e):

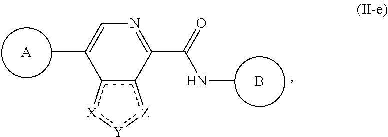

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-f):

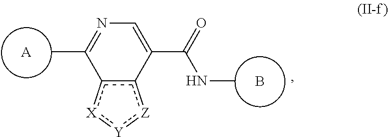

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}, each R^{A1} is hydrogen or C₁-C₆-alkyl; and x is 0, 1, or 2.

In some embodiments, the compound of Formula (II) is a compound of Formula (II-g):

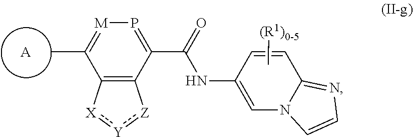

or a pharmaceutically acceptable salt, solvate, hydrate, tautomer, or stereoisomer thereof, A is cycloalkyl, heterocyclyl, aryl, or heteroaryl, each of which is optionally substituted with one or more R¹; M and P are each independently C(R²) or N; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶, each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-h):

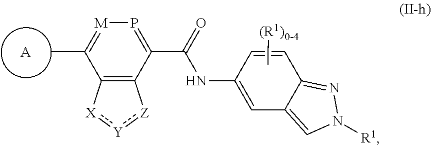

or a pharmaceutically acceptable salt, solvate, hydrate, tautomer, or stereoisomer thereof, A is cycloalkyl, heterocyclyl, aryl, or heteroaryl, each of which is optionally substituted with one or more R¹; M and P are each independently C(R²) or N; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments of Formula (II),

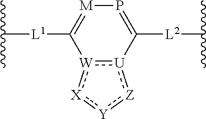

is selected from

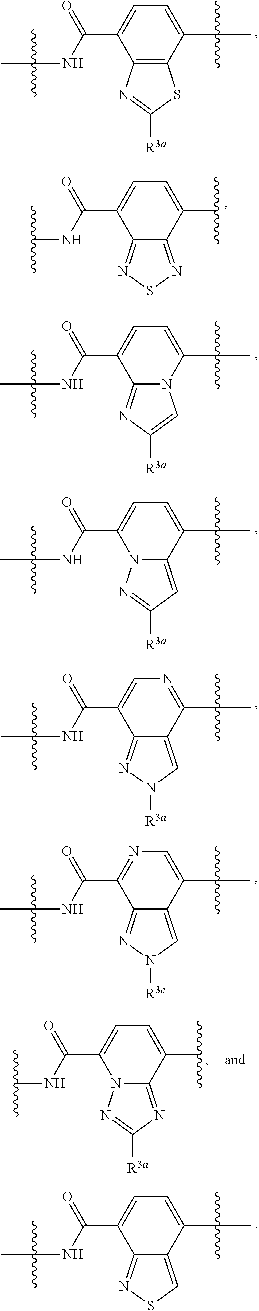

In some embodiments,

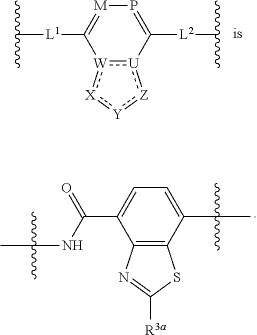

In some embodiments,

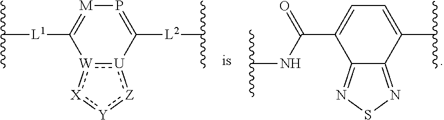

In some embodiments,

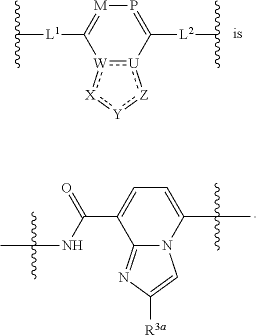

In some embodiments,

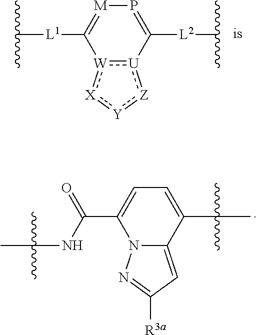

In some embodiments,

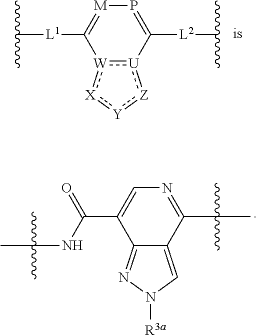

In some embodiments,

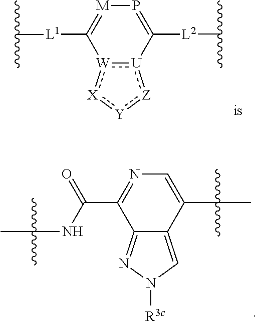

In some embodiments,

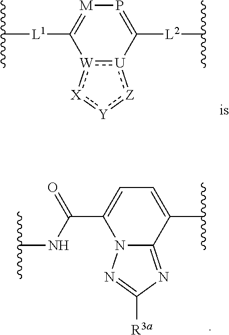

In some embodiments,

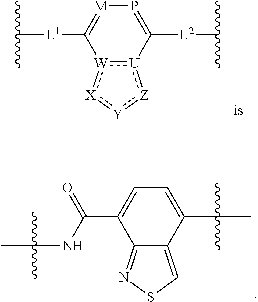

In some embodiments, the compound of Formula (II) is a compound of Formula (II-b):

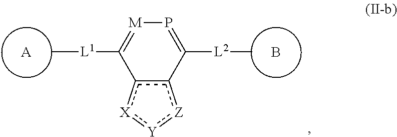

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; M and P are each independently C(R²) or N; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}, each R^{A1} is hydrogen or C₁-C₆-alkyl; and x is 0, 1, or 2.

In some embodiments, the compound of Formula (II) is a compound of Formula (II-c):

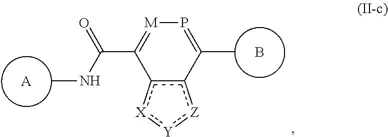

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; M and P are each independently C(R²) or N; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)R^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-d):

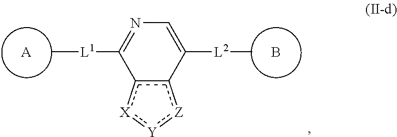

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}, each R^{A1} is hydrogen or C₁-C₆-alkyl; and x is 0, 1, or 2.

In some embodiments, the compound of Formula (II) is a compound of Formula (II-e):

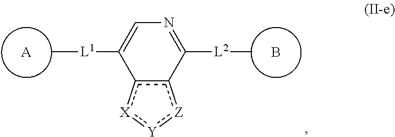

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-f):

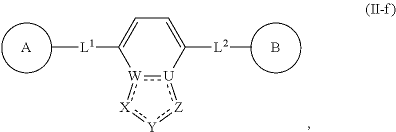

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; U and W are each independently C or N; X, Y, and Z are each independently C(R^{3a}), N, N(R^{3c}) or S, wherein at least one of X, Y, and Z is N or N(R^{3c}), and the bonds in the ring comprising U, W, X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-g):

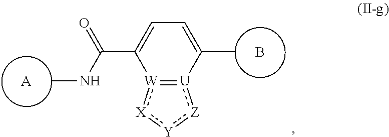

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; U and W are each independently C or N; X, Y, and Z are each independently C(R^{3a}), N, N(R^{3c}) or S, wherein at least one of X, Y, and Z is N or N(R^{3c}), and the bonds in the ring comprising U, W, X, Y, and Z may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-h):

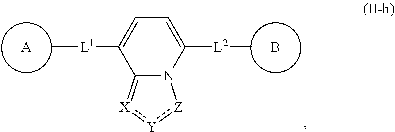

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-i):

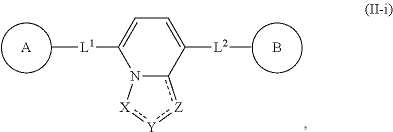

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R¹, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}, each R^{A1} is hydrogen or C₁-C₆-alkyl; and x is 0, 1, or 2.

In some embodiments, the compound of Formula (II) is a compound of Formula (II-j):

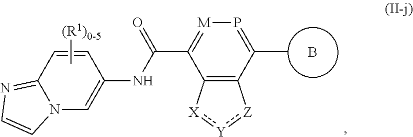

or a pharmaceutically acceptable salt, solvate, hydrate, tautomer, or stereoisomer thereof, B is cycloalkyl, heterocyclyl, aryl, or heteroaryl, each of which is optionally substituted with one or more R¹; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; M and P are each independently C(R²) or N; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}, each R^{A1} is hydrogen or C₁-C₆-alkyl; and x is 0, 1, or 2.

In some embodiments, the compound of Formula (II) is a compound of Formula (II-k):

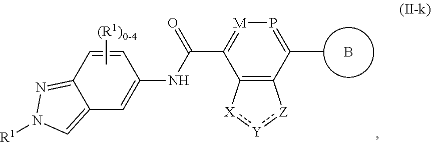

or a pharmaceutically acceptable salt, solvate, hydrate, tautomer, or stereoisomer thereof, B is cycloalkyl, heterocyclyl, aryl, or heteroaryl, each of which is optionally substituted with one or more R¹; X, Y, and Z are each independently C(R^{3a}), N, N(R^{3c}) or S, wherein at least one of X, Y, and Z is N or N(R^{3c}), and the bonds in the ring comprising X, Y, and Z may be single or double bonds as valency permits; M and P are each independently C(R²) or N; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}, each R^{A1} is hydrogen or C₁-C₆-alkyl; and x is 0, 1, or 2.

In some embodiments, the compound of Formula (II) is a compound of Formula (II-l):

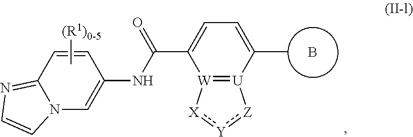

or a pharmaceutically acceptable salt, solvate, hydrate, tautomer, or stereoisomer thereof, B is cycloalkyl, heterocyclyl, aryl, or heteroaryl, each of which is optionally substituted with one or more R¹; U and W are each independently C or N; X, Y, and Z are each independently C(R^{3a}), N, N(R^{3c}) or S, wherein at least one of X, Y, and Z is N or N(R^{3c}), and the bonds in the ring comprising U, W, X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

In some embodiments, the compound of Formula (II) is a compound of Formula (II-m):

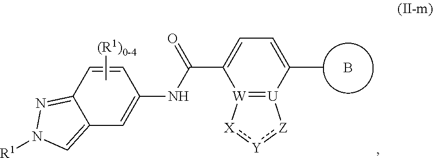

or a pharmaceutically acceptable salt, solvate, hydrate, tautomer, or stereoisomer thereof, B is cycloalkyl, heterocyclyl, aryl, or heteroaryl, each of which is optionally substituted with one or more R¹; U and W are each independently C or N; X, Y, and Z are each independently C(R^{3a}), N, N(R^{3c}) or S, wherein at least one of X, Y, and Z is N or N(R^{3c}), and the bonds in the ring comprising U, W, X, Y, and Z may be single or double bonds as valency permits; each of L¹ and L² is independently absent, C₁-C₆-alkylene, C₁-C₆-heteroalkylene, —O—, —C(O)—, —N(R⁴)—, —N(R⁴)C(O)—, or —C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R^{3a} is hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)OR^{D}, or —S(O)ₓR^{D}, or —C(O)R^{D}; R^{3c} is hydrogen, C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —S(O)ₓR^{D}, or —C(O)R^{D}; each R⁴ is independently hydrogen, C₁-C₆-alkyl, or C₁-C₆-haloalkyl; each R⁵ is independently hydrogen, C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹; each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A1} is hydrogen or C₁-C₆-alkyl; and x is 0, 1, or 2.

TABLE 1
Exemplary compounds of Formula (I) and (II)

| Cmpd | |
|---|---|
| No. | Structure |
| 100 | 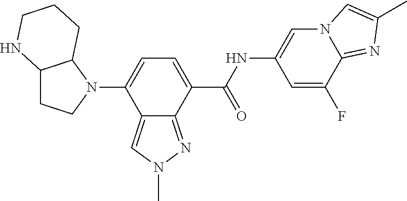 [SMILES: Cc1cn2cc(NC(=O)c3ccc(N4CCC5NCCCC54)c4cn(C)nc34)cc(F)c2n1] |
| 101 | 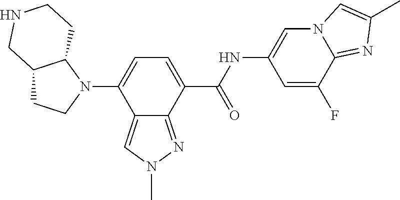 [SMILES: Cc1cn2cc(NC(=O)c3ccc(N4CC[C@@H]5CNCC[C@@H]54)c4cn(C)nc34)cc(F)c2n1] |
| 102 | 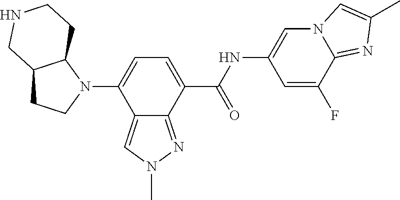 [SMILES: Cc1cn2cc(NC(=O)c3ccc(N4CC[C@H]5CNCC[C@H]54)c4cn(C)nc34)cc(F)c2n1] |
| 103 | 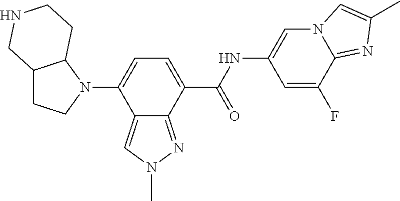 [SMILES: Cc1cn2cc(NC(=O)c3ccc(N4CCC5CNCCC54)c4cn(C)nc34)cc(F)c2n1] |
| 104 | 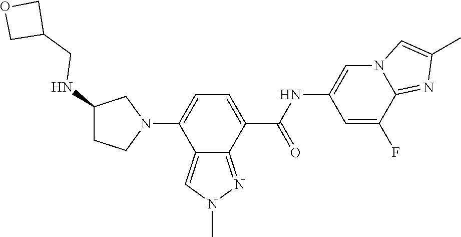 [SMILES: Cc1cn2cc(NC(=O)c3ccc(N4CC[C@@H](NCC5COC5)C4)c4cn(C)nc34)cc(F)c2n1] |
| 105 | 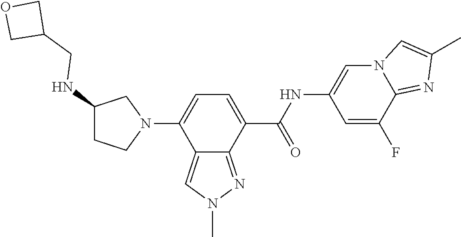 [SMILES: Cc1cn2cc(NC(=O)c3ccc(N4CC[C@@H](NCC5COC5)C4)c4cn(C)nc34)cc(F)c2n1] |
| 106 | 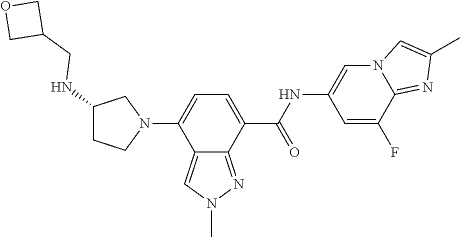 [SMILES: Cc1cn2cc(NC(=O)c3ccc(N4CC[C@H](NCC5COC5)C4)c4cn(C)nc34)cc(F)c2n1] |
| 107 | 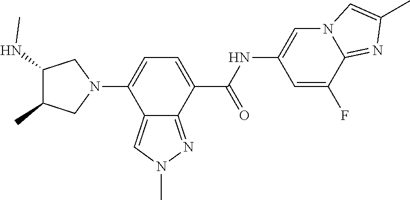 |
| 108 | 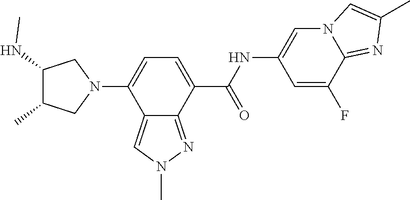 |
| 109 | 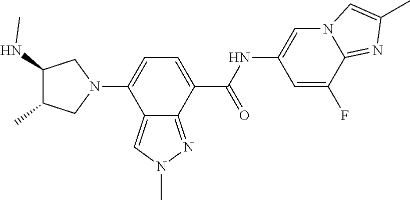 |
| 110 | 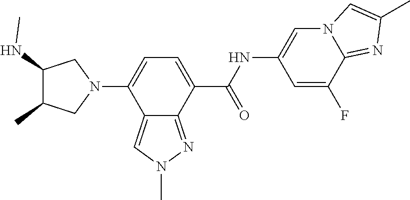 |
| 111 | 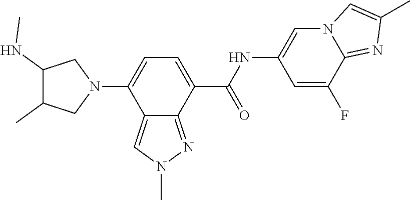 |
| 112 | 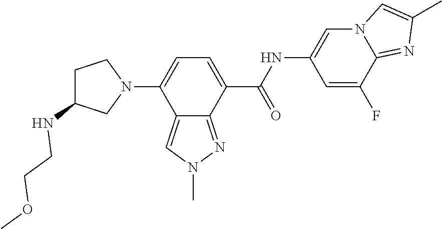 |
| 113 | 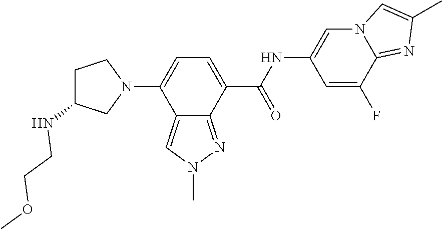 |
| 114 | 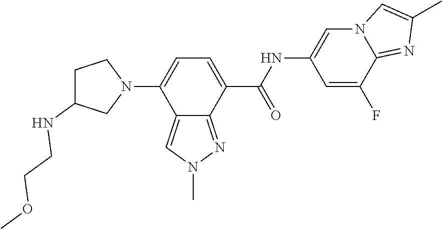 |
| 115 | 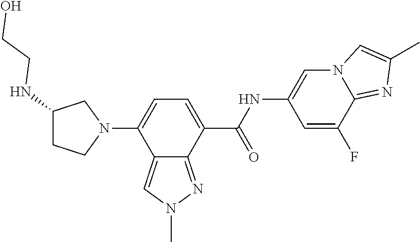 [SMILES: Cc1cn2cc(NC(=O)c3ccc(N4CC[C@H](NCCO)C4)c4cn(C)nc34)cc(F)c2n1] |
| 116 | 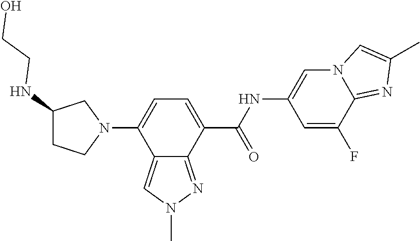 [SMILES: Cc1cn2cc(NC(=O)c3ccc(N4CC[C@@H](NCCO)C4)c4cn(C)nc34)cc(F)c2n1] |
| 117 | 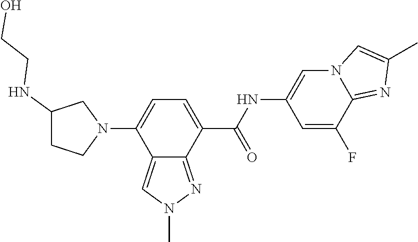 [SMILES: Cc1cn2cc(NC(=O)c3ccc(N4CCC(NCCO)C4)c4cn(C)nc34)cc(F)c2n1] |
| 118 | 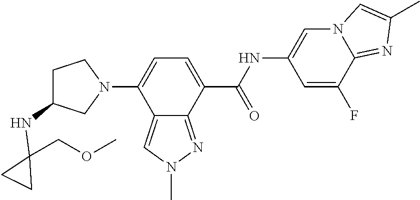 [SMILES: COCC1(N[C@H]2CCN(c3ccc(C(=O)Nc4cc(F)c5nc(C)cn5c4)c4nn(C)cc34)C2)CC1] |
| 119 | 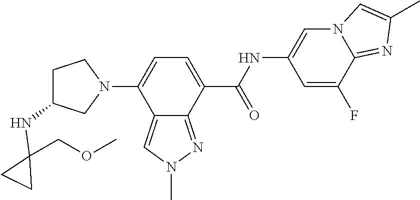 [SMILES: COCC1(N[C@@H]2CCN(c3ccc(C(=O)Nc4cc(F)c5nc(C)cn5c4)c4nn(C)cc34)C2)CC1] |
| 120 | 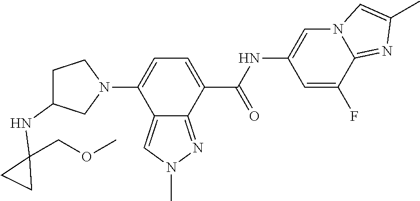 [SMILES: COCC1(NC2CCN(c3ccc(C(=O)Nc4cc(F)c5nc(C)cn5c4)c4nn(C)cc34)C2)CC1] |
| 121 | 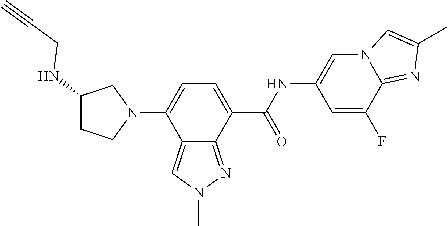 [SMILES: C#CCN[C@H]1CCN(c2ccc(C(=O)Nc3cc(F)c4nc(C)cn4c3)c3nn(C)cc23)C1] |
| 122 | 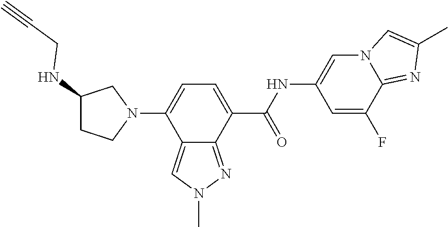 [SMILES: C#CCN[C@@H]1CCN(c2ccc(C(=O)Nc3cc(F)c4nc(C)cn4c3)c3nn(C)cc23)C1] |
| 123 | 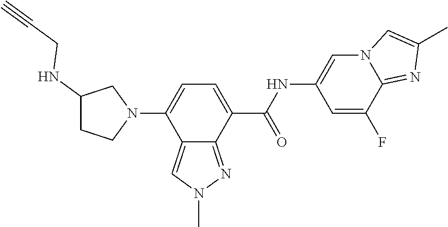 [SMILES: C#CCNC1CCN(c2ccc(C(=O)Nc3cc(F)c4nc(C)cn4c3)c3nn(C)cc23)C1] |
| 124 | 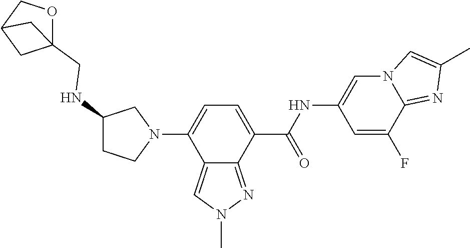 [SMILES: Cc1cn2cc(NC(=O)c3ccc(N4CC[C@@H](NCC56CC(CO5)C6)C4)c4cn(C)nc34)cc(F)c2n1] |
| 125 | 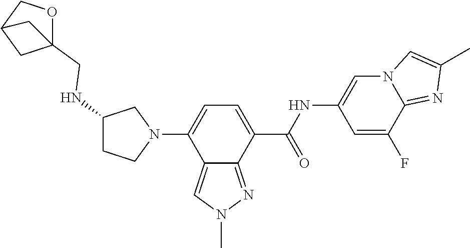 [SMILES: Cc1cn2cc(NC(=O)c3ccc(N4CC[C@H](NCC56CC(CO5)C6)C4)c4cn(C)nc34)cc(F)c2n1] |
| 126 | 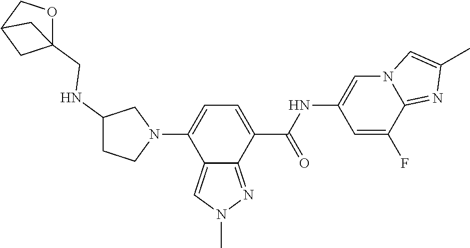 [SMILES: Cc1cn2cc(NC(=O)c3ccc(N4CCC(NCC56CC(CO5)C6)C4)c4cn(C)nc34)cc(F)c2n1] |
| 127 | 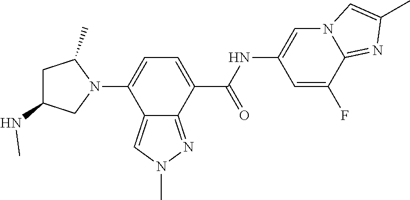 [SMILES: CN[C@H]1C[C@H](C)N(c2ccc(C(=O)Nc3cc(F)c4nc(C)cn4c3)c3nn(C)cc23)C1] |
| 128 | 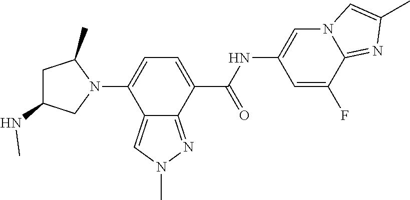 [SMILES: CN[C@H]1C[C@@H](C)N(c2ccc(C(=O)Nc3cc(F)c4nc(C)cn4c3)c3nn(C)cc23)C1] |
| 129 | 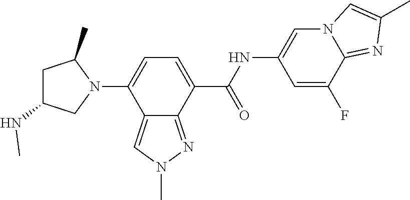 [SMILES: CN[C@@H]1C[C@@H](C)N(c2ccc(C(=O)Nc3cc(F)c4nc(C)cn4c3)c3nn(C)cc23)C1] |
| 130 | 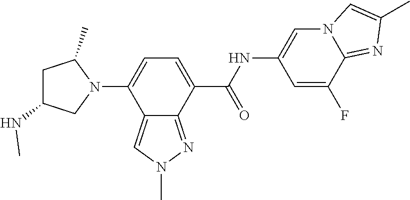 [SMILES: CN[C@@H]1C[C@H](C)N(c2ccc(C(=O)Nc3cc(F)c4nc(C)cn4c3)c3nn(C)cc23)C1] |
| 131 | 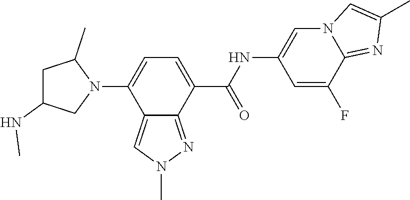 [SMILES: CNC1CC(C)N(c2ccc(C(=O)Nc3cc(F)c4nc(C)cn4c3)c3nn(C)cc23)C1] |
| 132 | 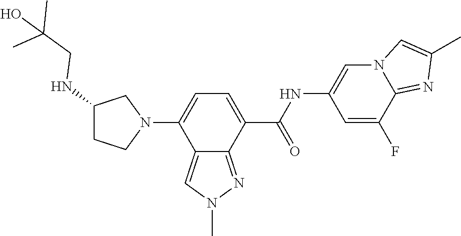 [SMILES: Cc1cn2cc(NC(=O)c3ccc(N4CC[C@H](NCC(C)(C)O)C4)c4cn(C)nc34)cc(F)c2n1] |
| 133 | 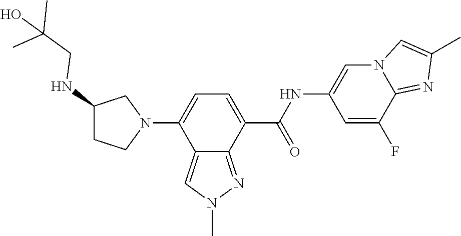 [SMILES: Cc1cn2cc(NC(=O)c3ccc(N4CC[C@@H](NCC(C)(C)O)C4)c4cn(C)nc34)cc(F)c2n1] |
| 134 | 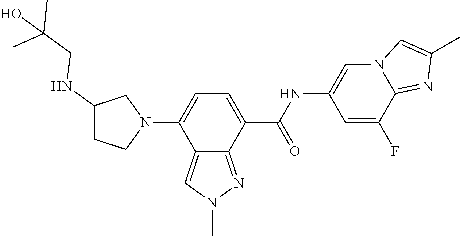 [SMILES: Cc1cn2cc(NC(=O)c3ccc(N4CCC(NCC(C)(C)O)C4)c4cn(C)nc34)cc(F)c2n1] |
| 135 | 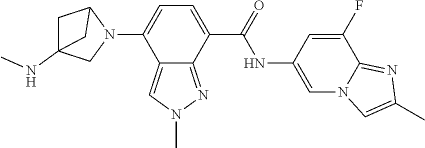 |
| 136 | 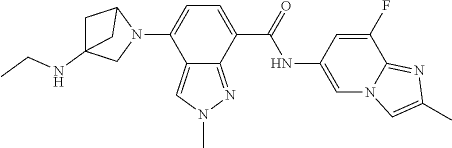 [SMILES: CCNC12CC(C1)N(c1ccc(C(=O)Nc3cc(F)c4nc(C)cn4c3)c3nn(C)cc13)C2] |
| 137 | 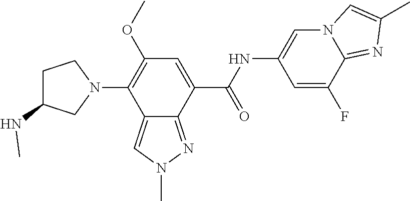 [SMILES: CN[C@H]1CCN(c2c(OC)cc(C(=O)Nc3cc(F)c4nc(C)cn4c3)c3nn(C)cc23)C1] |
| 138 | 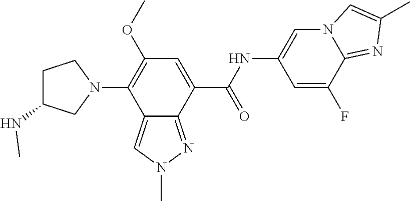 [SMILES: CN[C@@H]1CCN(c2c(OC)cc(C(=O)Nc3cc(F)c4nc(C)cn4c3)c3nn(C)cc23)C1] |
| 139 | 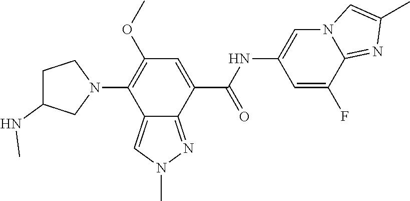 [SMILES: CNC1CCN(c2c(OC)cc(C(=O)Nc3cc(F)c4nc(C)cn4c3)c3nn(C)cc23)C1] |
| 140 | 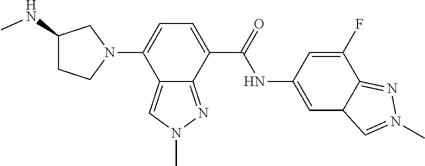 |
| 141 | 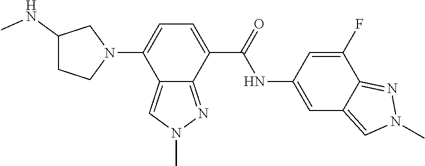 |
| 142 | 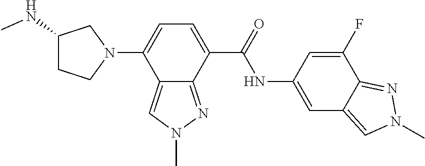 |
| 143 | 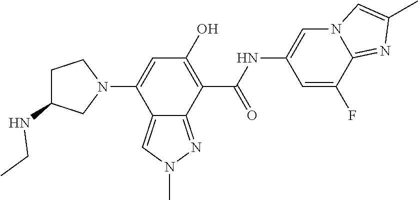 [SMILES: CCN[C@H]1CCN(c2cc(O)c(C(=O)Nc3cc(F)c4nc(C)cn4c3)c3nn(C)cc23)C1] |
| 144 | 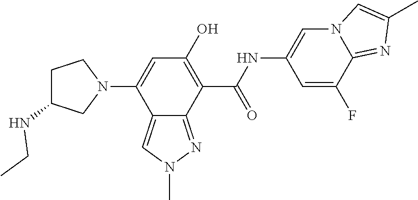 [SMILES: CCN[C@@H]1CCN(c2cc(O)c(C(=O)Nc3cc(F)c4nc(C)cn4c3)c3nn(C)cc23)C1] |
| 145 | 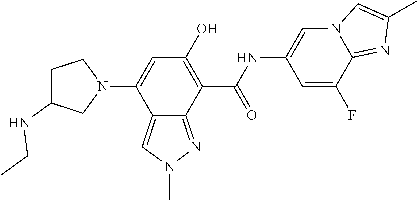 [SMILES: CCNC1CCN(c2cc(O)c(C(=O)Nc3cc(F)c4nc(C)cn4c3)c3nn(C)cc23)C1] |
| 146 | 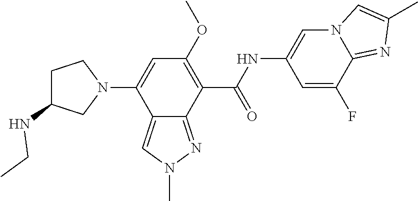 [SMILES: CCN[C@H]1CCN(c2cc(OC)c(C(=O)Nc3cc(F)c4nc(C)cn4c3)c3nn(C)cc23)C1] |
| 147 | 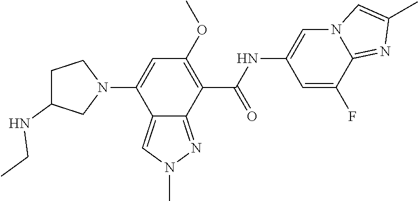 [SMILES: CCNC1CCN(c2cc(OC)c(C(=O)Nc3cc(F)c4nc(C)cn4c3)c3nn(C)cc23)C1] |
| 148 | 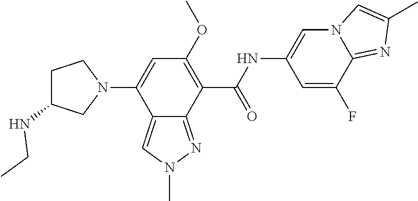 [SMILES: CCN[C@@H]1CCN(c2cc(OC)c(C(=O)Nc3cc(F)c4nc(C)cn4c3)c3nn(C)cc23)C1] |
| 149 | 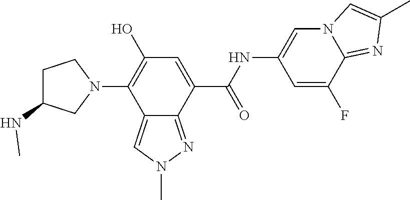 [SMILES: CN[C@H]1CCN(c2c(O)cc(C(=O)Nc3cc(F)c4nc(C)cn4c3)c3nn(C)cc23)C1] |
| 150 | 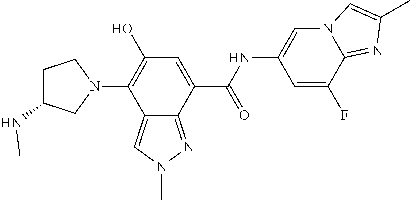 [SMILES: CN[C@@H]1CCN(c2c(O)cc(C(=O)Nc3cc(F)c4nc(C)cn4c3)c3nn(C)cc23)C1] |
| 151 | 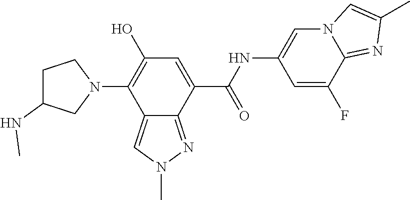 [SMILES: CNC1CCN(c2c(O)cc(C(=O)Nc3cc(F)c4nc(C)cn4c3)c3nn(C)cc23)C1] |
| 152 | 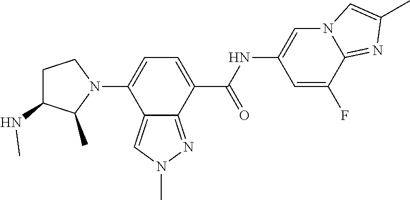 [SMILES: CN[C@H]1CCN(c2ccc(C(=O)Nc3cc(F)c4nc(C)cn4c3)c3nn(C)cc23)[C@H]1C] |
| 153 | 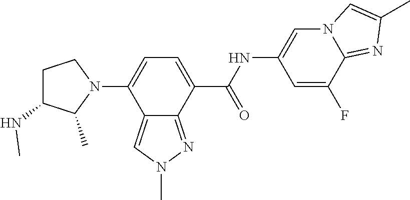 [SMILES: CN[C@@H]1CCN(c2ccc(C(=O)Nc3cc(F)c4nc(C)cn4c3)c3nn(C)cc23)[C@@H]1C] |
| 154 | 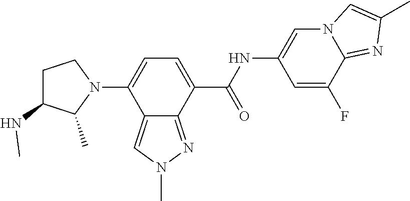 [SMILES: CN[C@H]1CCN(c2ccc(C(=O)Nc3cc(F)c4nc(C)cn4c3)c3nn(C)cc23)[C@@H]1C] |
| 155 | 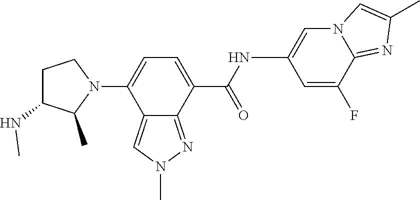 [SMILES: CN[C@@H]1CCN(c2ccc(C(=O)Nc3cc(F)c4nc(C)cn4c3)c3nn(C)cc23)[C@H]1C] |
| 156 | 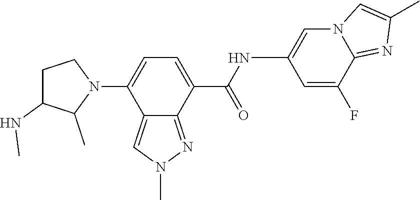 [SMILES: CNC1CCN(c2ccc(C(=O)Nc3cc(F)c4nc(C)cn4c3)c3nn(C)cc23)C1C] |
| 157 | 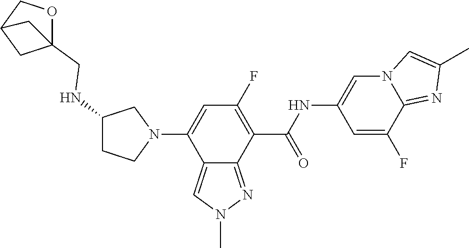 [SMILES: Cc1cn2cc(NC(=O)c3c(F)cc(N4CC[C@H](NCC56CC(CO5)C6)C4)c4cn(C)nc34)cc(F)c2n1] |
| 158 | 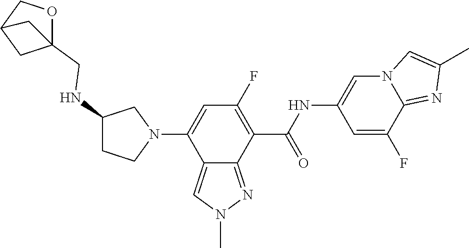 [SMILES: Cc1cn2cc(NC(=O)c3c(F)cc(N4CC[C@@H](NCC56CC(CO5)C6)C4)c4cn(C)nc34)cc(F)c2n1] |
| 159 | 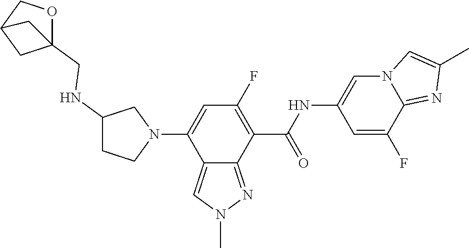 [SMILES: Cc1cn2cc(NC(=O)c3c(F)cc(N4CCC(NCC56CC(CO5)C6)C4)c4cn(C)nc34)cc(F)c2n1] |
| 160 | 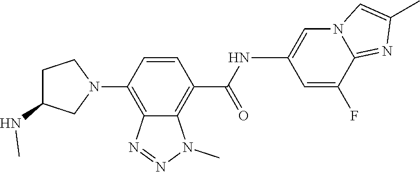 [SMILES: CN[C@H]1CCN(c2ccc(C(=O)Nc3cc(F)c4nc(C)cn4c3)c3c2nnn3C)C1] |
| 161 | 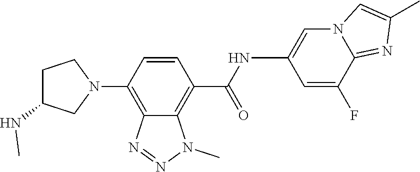 [SMILES: CN[C@@H]1CCN(c2ccc(C(=O)Nc3cc(F)c4nc(C)cn4c3)c3c2nnn3C)C1] |
| 162 | 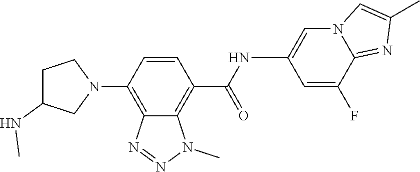 [SMILES: CNC1CCN(c2ccc(C(=O)Nc3cc(F)c4nc(C)cn4c3)c3c2nnn3C)C1] |
| 163 | 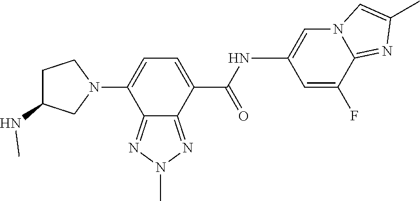 [SMILES: CN[C@H]1CCN(c2ccc(C(=O)Nc3cc(F)c4nc(C)cn4c3)c3nn(C)nc23)C1] |
| 164 | 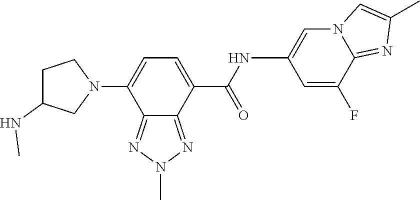 [SMILES: CNC1CCN(c2ccc(C(=O)Nc3cc(F)c4nc(C)cn4c3)c3nn(C)nc23)C1] |
| 165 | 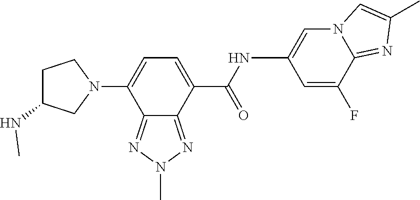 [SMILES: CN[C@@H]1CCN(c2ccc(C(=O)Nc3cc(F)c4nc(C)cn4c3)c3nn(C)nc23)C1] |
| 166 | 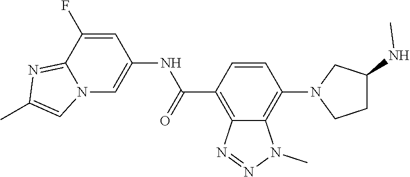 [SMILES: CN[C@H]1CCN(c2ccc(C(=O)Nc3cc(F)c4nc(C)cn4c3)c3nnn(C)c23)C1] |
| 167 | 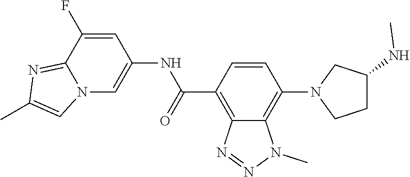 [SMILES: CN[C@@H]1CCN(c2ccc(C(=O)Nc3cc(F)c4nc(C)cn4c3)c3nnn(C)c23)C1] |
| 168 | 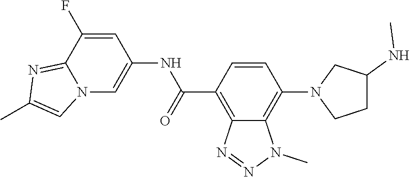 [SMILES: CNC1CCN(c2ccc(C(=O)Nc3cc(F)c4nc(C)cn4c3)c3nnn(C)c23)C1] |
| 169 | 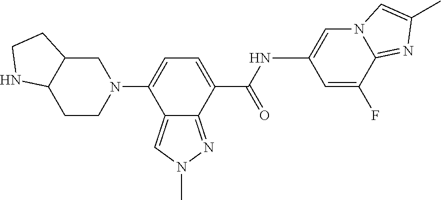 [SMILES: Cc1cn2cc(NC(=O)c3ccc(N4CCC5NCCC5C4)c4cn(C)nc34)cc(F)c2n1] |
| 170 | 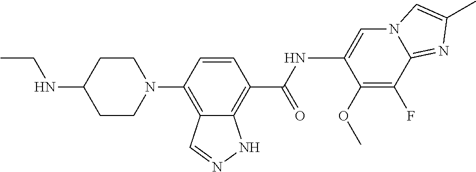 [SMILES: CCNC1CCN(c2ccc(C(=O)Nc3cn4cc(C)nc4c(F)c3OC)c3[nH]ncc23)CC1] |
| 171 | 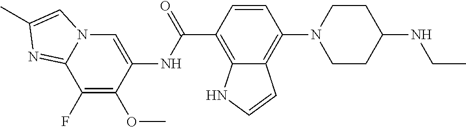 [SMILES: CCNC1CCN(c2ccc(C(=O)Nc3cn4cc(C)nc4c(F)c3OC)c3[nH]ccc23)CC1] |
| 172 | 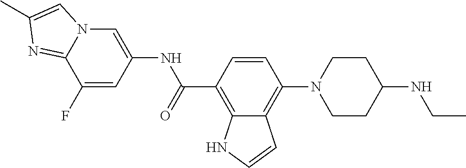 [SMILES: CCNC1CCN(c2ccc(C(=O)Nc3cc(F)c4nc(C)cn4c3)c3[nH]ccc23)CC1] |
| 173 | 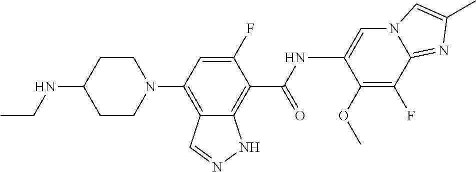 [SMILES: CCNC1CCN(c2cc(F)c(C(=O)Nc3cn4cc(C)nc4c(F)c3OC)c3[nH]ncc23)CC1] |
| 174 | 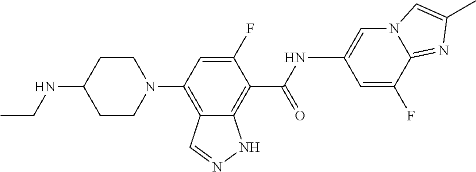 [SMILES: CCNC1CCN(c2cc(F)c(C(=O)Nc3cc(F)c4nc(C)cn4c3)c3[nH]ncc23)CC1] |
| 175 | 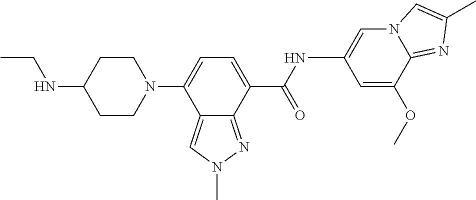 [SMILES: CCNC1CCN(c2ccc(C(=O)Nc3cc(OC)c4nc(C)cn4c3)c3nn(C)cc23)CC1] |
| 176 | 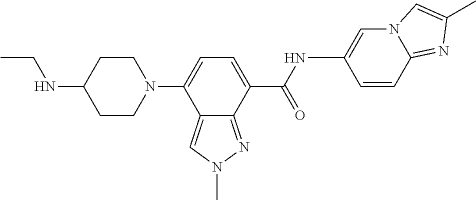 [SMILES: CCNC1CCN(c2ccc(C(=O)Nc3ccc4nc(C)cn4c3)c3nn(C)cc23)CC1] |
| 177 | 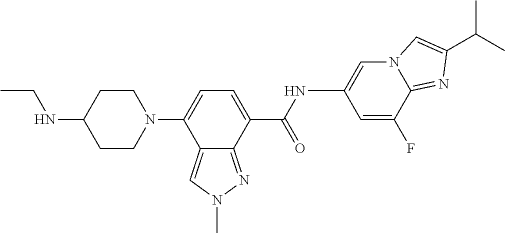 [SMILES: CCNC1CCN(c2ccc(C(=O)Nc3cc(F)c4nc(C(C)C)cn4c3)c3nn(C)cc23)CC1] |
| 178 | 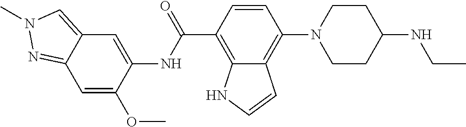 [SMILES: CCNC1CCN(c2ccc(C(=O)Nc3cc4cn(C)nc4cc3OC)c3[nH]ccc23)CC1] |
| 179 | 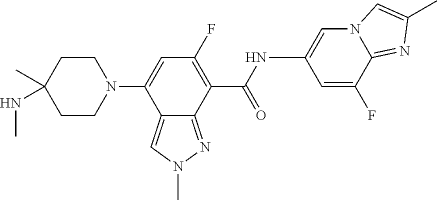 |
| 180 | 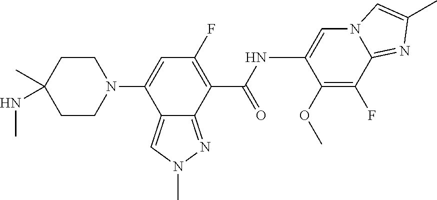 |
| 181 | 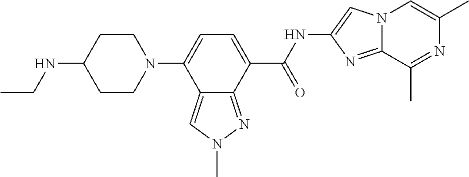 [SMILES: CCNC1CCN(c2ccc(C(=O)Nc3cn4cc(C)nc(C)c4n3)c3nn(C)cc23)CC1] |
| 182 | 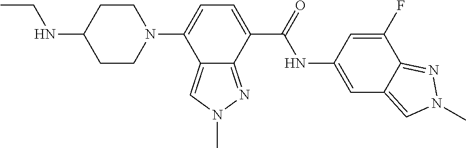 [SMILES: CCNC1CCN(c2ccc(C(=O)Nc3cc(F)c4nn(C)cc4c3)c3nn(C)cc23)CC1] |
| 183 | 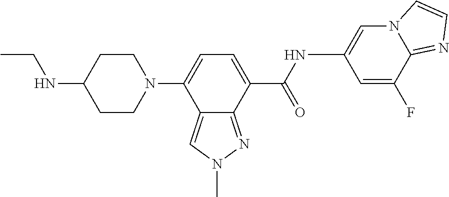 [SMILES: CCNC1CCN(c2ccc(C(=O)Nc3cc(F)c4nccn4c3)c3nn(C)cc23)CC1] |
| 184 | 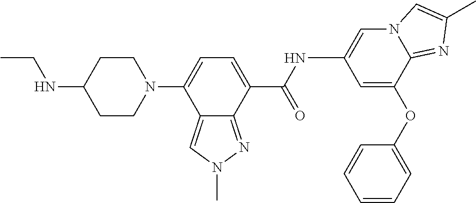 [SMILES: CCNC1CCN(c2ccc(C(=O)Nc3cc(Oc4ccccc4)c4nc(C)cn4c3)c3nn(C)cc23)CC1] |
| 185 | 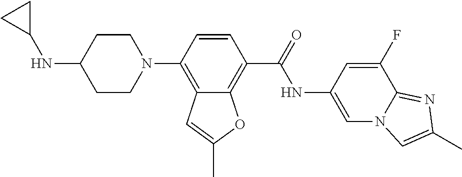 [SMILES: Cc1cn2cc(NC(=O)c3ccc(N4CCC(NC5CC5)CC4)c4cc(C)oc34)cc(F)c2n1] |
| 186 | 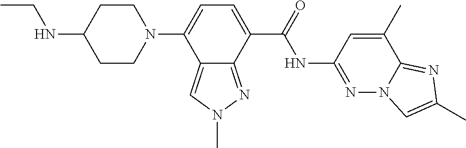 [SMILES: CCNC1CCN(c2ccc(C(=O)Nc3cc(C)c4nc(C)cn4n3)c3nn(C)cc23)CC1] |
| 187 | 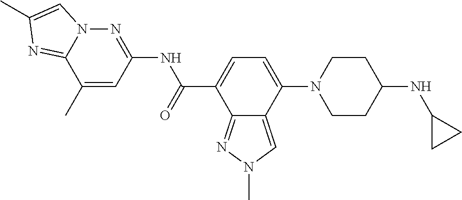 [SMILES: Cc1cn2nc(NC(=O)c3ccc(N4CCC(NC5CC5)CC4)c4cn(C)nc34)cc(C)c2n1] |
| 188 | 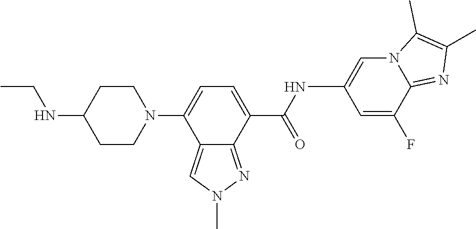 [SMILES: CCNC1CCN(c2ccc(C(=O)Nc3cc(F)c4nc(C)c(C)n4c3)c3nn(C)cc23)CC1] |
| 189 | 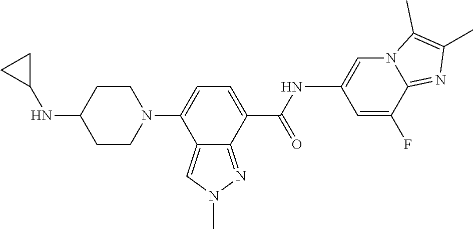 [SMILES: Cc1nc2c(F)cc(NC(=O)c3ccc(N4CCC(NC5CC5)CC4)c4cn(C)nc34)cn2c1C] |
| 190 | 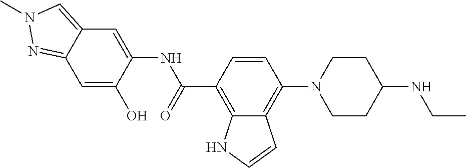 [SMILES: CCNC1CCN(c2ccc(C(=O)Nc3cc4cn(C)nc4cc3O)c3[nH]ccc23)CC1] |
| 191 | 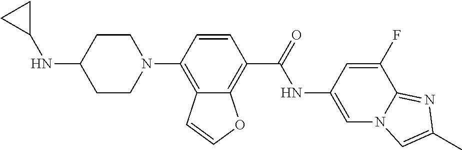 [SMILES: Cc1cn2cc(NC(=O)c3ccc(N4CCC(NC5CC5)CC4)c4ccoc34)cc(F)c2n1] |
| 192 | 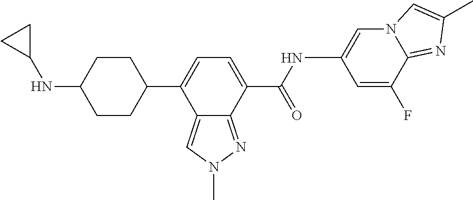 [SMILES: Cc1cn2cc(NC(=O)c3ccc(C4CCC(NC5CC5)CC4)c4cn(C)nc34)cc(F)c2n1] |
| 193 | 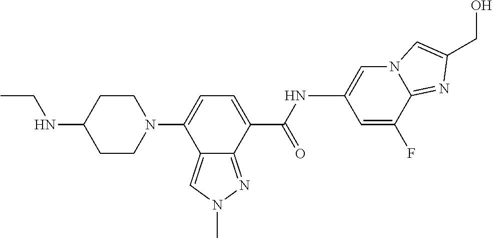 [SMILES: CCNC1CCN(c2ccc(C(=O)Nc3cc(F)c4nc(CO)cn4c3)c3nn(C)cc23)CC1] |
| 194 | 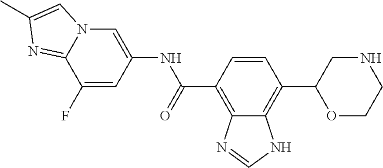 [SMILES: Cc1cn2cc(NC(=O)c3ccc(C4CNCCO4)c4[nH]cnc34)cc(F)c2n1] |
| 195 | 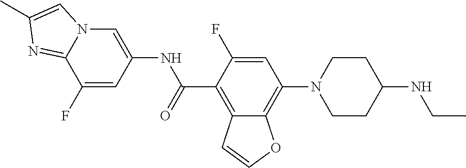 [SMILES: CCNC1CCN(c2cc(F)c(C(=O)Nc3cc(F)c4nc(C)cn4c3)c3ccoc23)CC1] |
| 196 | 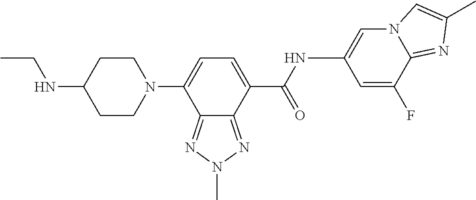 [SMILES: CCNC1CCN(c2ccc(C(=O)Nc3cc(F)c4nc(C)cn4c3)c3nn(C)nc23)CC1] |
| 197 | 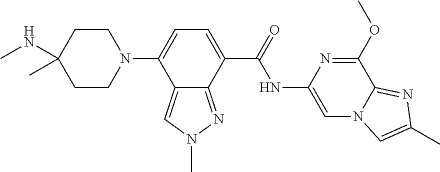 |
| 198 | 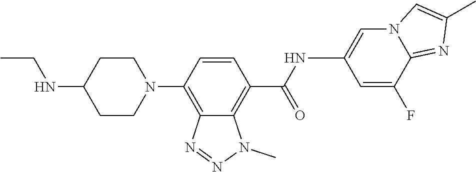 [SMILES: CCNC1CCN(c2ccc(C(=O)Nc3cc(F)c4nc(C)cn4c3)c3c2nnn3C)CC1] |
| 199 | 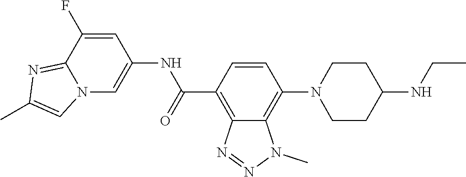 [SMILES: CCNC1CCN(c2ccc(C(=O)Nc3cc(F)c4nc(C)cn4c3)c3nnn(C)c23)CC1] |
| 200 | 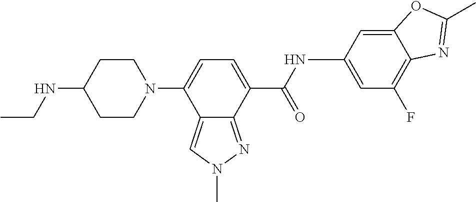 [SMILES: CCNC1CCN(c2ccc(C(=O)Nc3cc(F)c4nc(C)oc4c3)c3nn(C)cc23)CC1] |
| 201 | 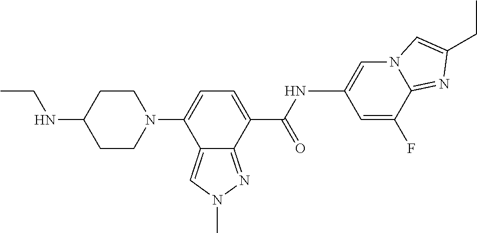 [SMILES: CCNC1CCN(c2ccc(C(=O)Nc3cc(F)c4nc(CC)cn4c3)c3nn(C)cc23)CC1] |
| 202 | 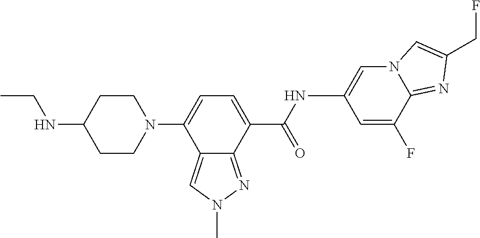 [SMILES: CCNC1CCN(c2ccc(C(=O)Nc3cc(F)c4nc(CF)cn4c3)c3nn(C)cc23)CC1] |
| 203 | 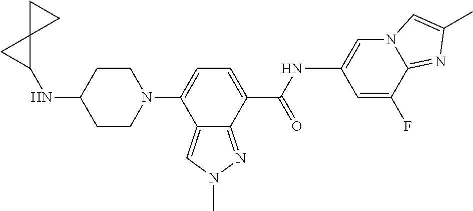 [SMILES: Cc1cn2cc(NC(=O)c3ccc(N4CCC(NC5CC56CC6)CC4)c4cn(C)nc34)cc(F)c2n1] |
| 204 | 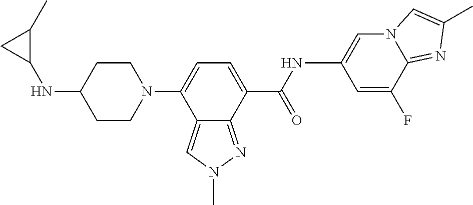 [SMILES: Cc1cn2cc(NC(=O)c3ccc(N4CCC(NC5CC5C)CC4)c4cn(C)nc34)cc(F)c2n1] |
| 205 | 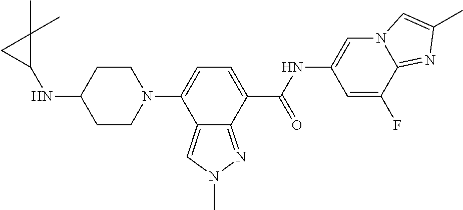 [SMILES: Cc1cn2cc(NC(=O)c3ccc(N4CCC(NC5CC5(C)C)CC4)c4cn(C)nc34)cc(F)c2n1] |
| 206 | 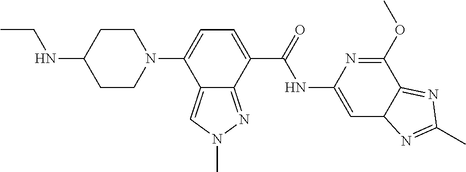 [SMILES: CCNC1CCN(c2ccc(C(=O)NC3=CC4N=C(C)N=C4C(OC)=N3)c3nn(C)cc23)CC1] |
| 207 | 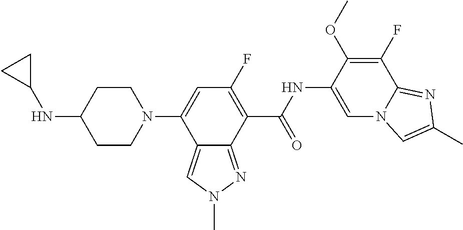 [SMILES: COc1c(NC(=O)c2c(F)cc(N3CCC(NC4CC4)CC3)c3cn(C)nc23)cn2cc(C)nc2c1F] |
| 208 | 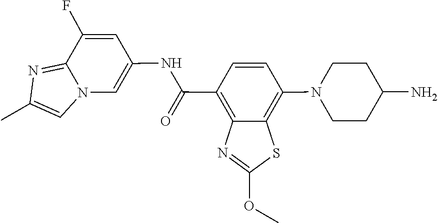 [SMILES: COc1nc2c(C(=O)Nc3cc(F)c4nc(C)cn4c3)ccc(N3CCC(N)CC3)c2s1] |
| 209 | 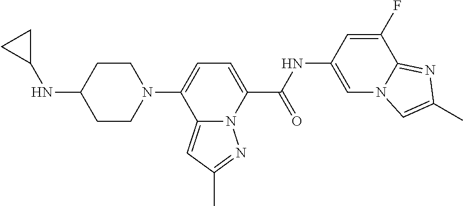 [SMILES: Cc1cn2cc(NC(=O)c3ccc(N4CCC(NC5CC5)CC4)c4cc(C)nn34)cc(F)c2n1] |
| 210 | 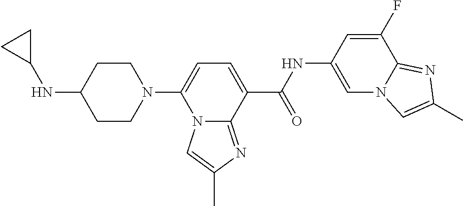 [SMILES: Cc1cn2cc(NC(=O)c3ccc(N4CCC(NC5CC5)CC4)n4cc(C)nc34)cc(F)c2n1] |
| 211 | 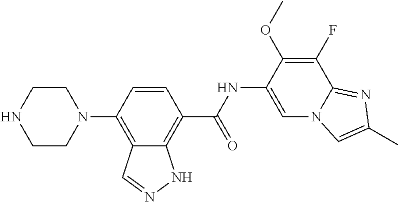 [SMILES: COc1c(NC(=O)c2ccc(N3CCNCC3)c3cn[nH]c23)cn2cc(C)nc2c1F] |
| 212 | 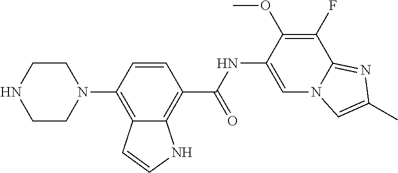 [SMILES: COc1c(NC(=O)c2ccc(N3CCNCC3)c3cc[nH]c23)cn2cc(C)nc2c1F] |
| 213 | 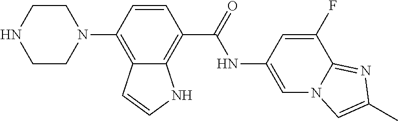 [SMILES: Cc1cn2cc(NC(=O)c3ccc(N4CCNCC4)c4cc[nH]c34)cc(F)c2n1] |
| 214 | 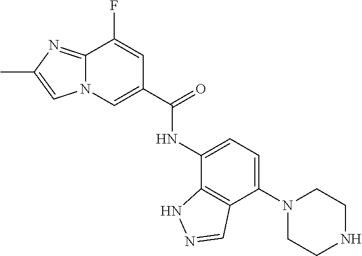 [SMILES: Cc1cn2cc(C(=O)Nc3ccc(N4CCNCC4)c4cn[nH]c34)cc(F)c2n1] |
| 215 | 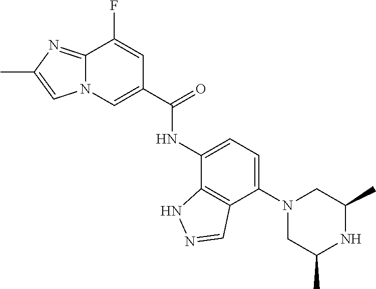 |
| 216 | 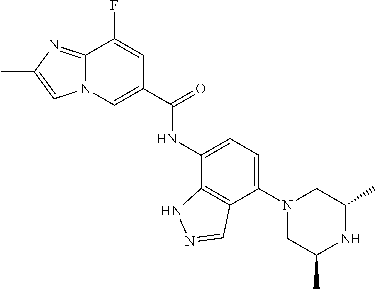 |
| 217 | 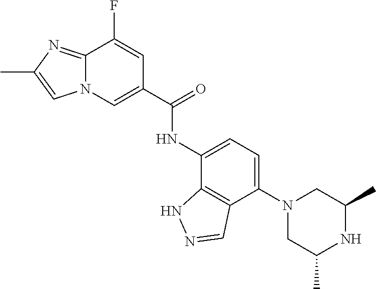 |
| 218 | 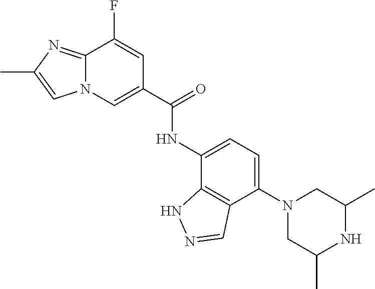 |
| 220 | 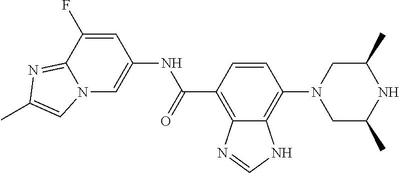 |
| 221 | 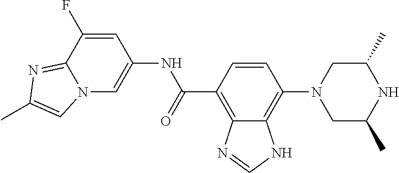 |
| 222 | 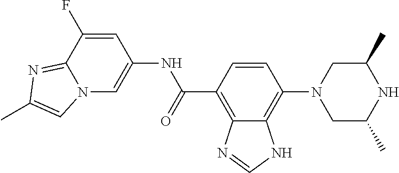 |
| 224 | 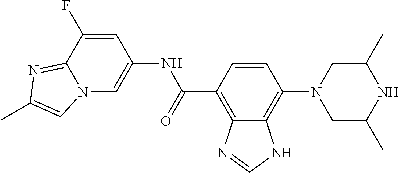 |
| 225 | 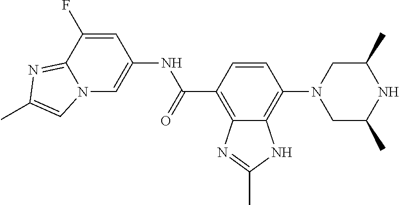 |
| 226 | 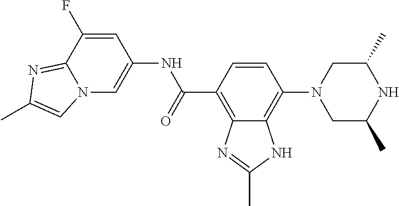 |
| 227 | 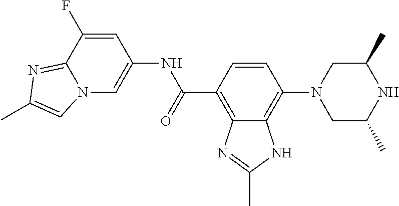 |
| 229 | 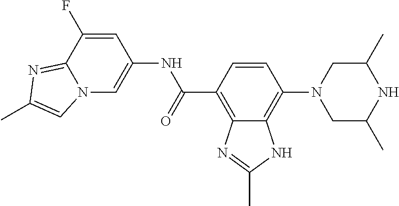 |
| 230 | 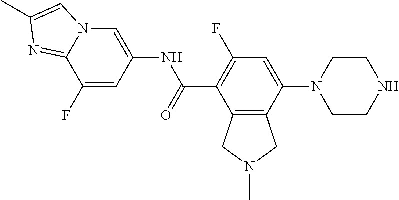 [SMILES: Cc1cn2cc(NC(=O)c3c(F)cc(N4CCNCC4)c4c3CN(C)C4)cc(F)c2n1] |
| 237 | 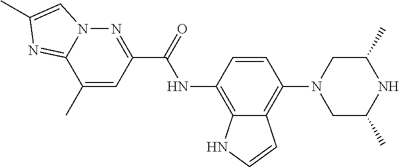 |
| 238 | 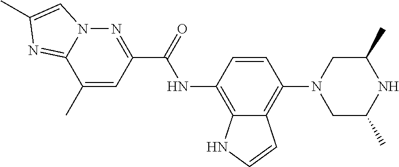 |
| 239 | 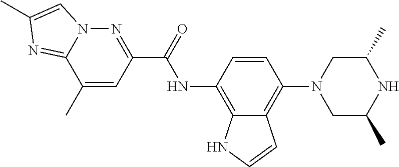 |
| 241 | 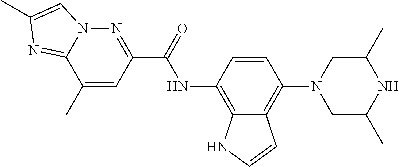 |
| 242 | 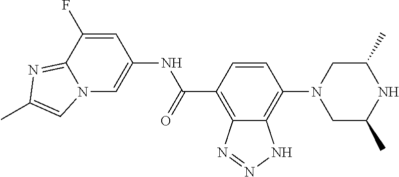 |
| 243 | 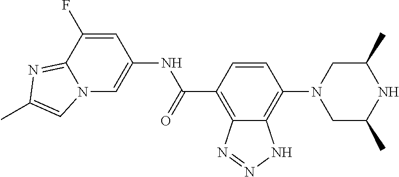 |
| 244 | 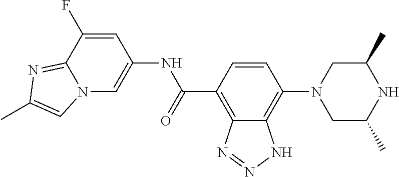 |
| 246 | 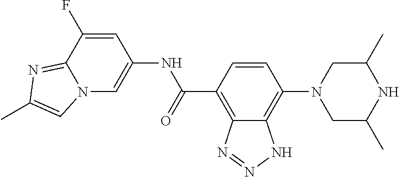 |
| 247 | 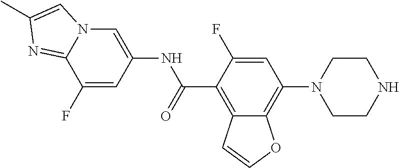 [SMILES: Cc1cn2cc(NC(=O)c3c(F)cc(N4CCNCC4)c4occc34)cc(F)c2n1] |
| 248 | 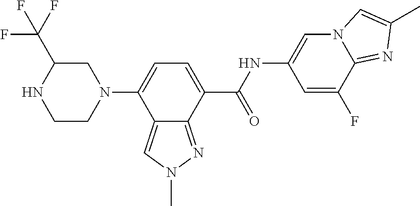 [SMILES: Cc1cn2cc(NC(=O)c3ccc(N4CCNC(C(F)(F)F)C4)c4cn(C)nc34)cc(F)c2n1] |
| 249 | 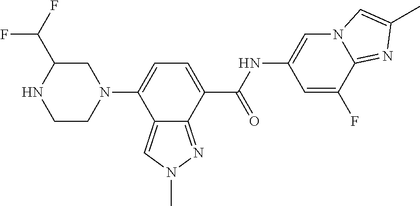 [SMILES: Cc1cn2cc(NC(=O)c3ccc(N4CCNC(C(F)F)C4)c4cn(C)nc34)cc(F)c2n1] |
| 250 | 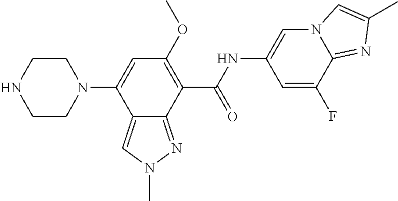 [SMILES: COc1cc(N2CCNCC2)c2cn(C)nc2c1C(=O)Nc1cc(F)c2nc(C)cn2c1] |
| 251 | 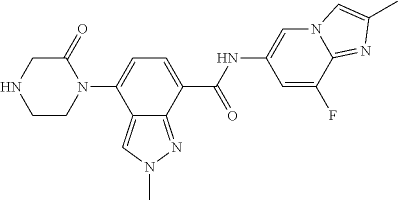 [SMILES: Cc1cn2cc(NC(=O)c3ccc(N4CCNCC4=O)c4cn(C)nc34)cc(F)c2n1] |
| 252 | 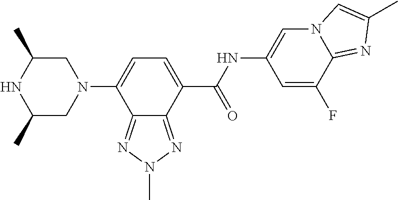 [SMILES: Cc1cn2cc(NC(=O)c3ccc(N4C[C@@H](C)N[C@@H](C)C4)c4nn(C)nc34)cc(F)c2n1] |
| 253 | 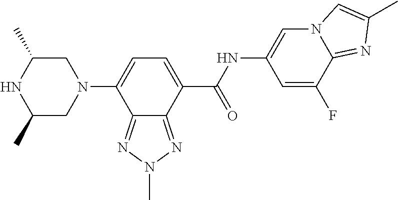 [SMILES: Cc1cn2cc(NC(=O)c3ccc(N4C[C@@H](C)N[C@H](C)C4)c4nn(C)nc34)cc(F)c2n1] |
| 254 | 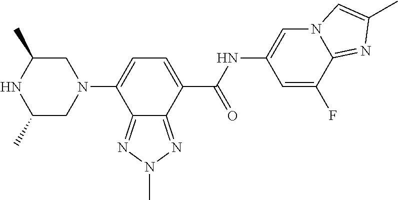 [SMILES: Cc1cn2cc(NC(=O)c3ccc(N4C[C@H](C)N[C@@H](C)C4)c4nn(C)nc34)cc(F)c2n1] |
| 256 | 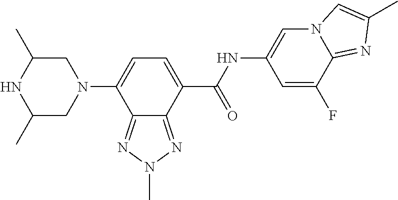 [SMILES: Cc1cn2cc(NC(=O)c3ccc(N4CC(C)NC(C)C4)c4nn(C)nc34)cc(F)c2n1] |
| 257 | 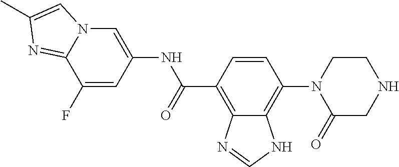 [SMILES: Cc1cn2cc(NC(=O)c3ccc(N4CCNCC4=O)c4[nH]cnc34)cc(F)c2n1] |
| 258 | 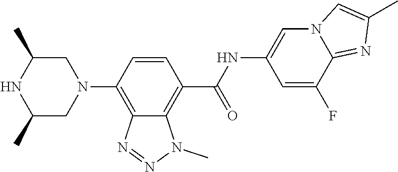 [SMILES: Cc1cn2cc(NC(=O)c3ccc(N4C[C@@H](C)N[C@@H](C)C4)c4nnn(C)c34)cc(F)c2n1] |
| 259 | 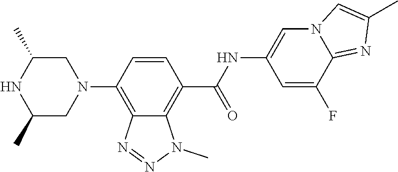 [SMILES: Cc1cn2cc(NC(=O)c3ccc(N4C[C@@H](C)N[C@H](C)C4)c4nnn(C)c34)cc(F)c2n1] |
| 260 | 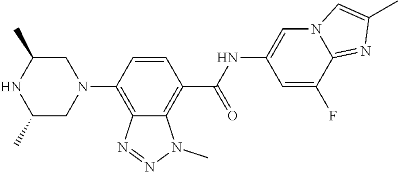 [SMILES: Cc1cn2cc(NC(=O)c3ccc(N4C[C@H](C)N[C@@H](C)C4)c4nnn(C)c34)cc(F)c2n1] |
| 261 | 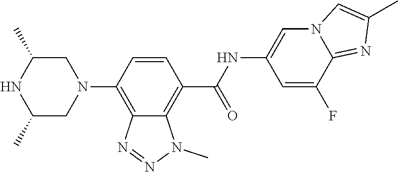 [SMILES: Cc1cn2cc(NC(=O)c3ccc(N4C[C@@H](C)N[C@@H](C)C4)c4nnn(C)c34)cc(F)c2n1] |
| 262 | 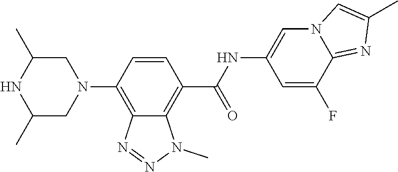 [SMILES: Cc1cn2cc(NC(=O)c3ccc(N4CC(C)NC(C)C4)c4nnn(C)c34)cc(F)c2n1] |
| 263 | 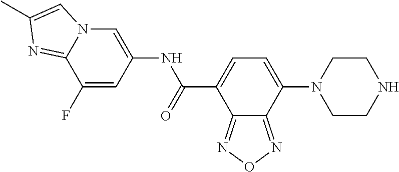 [SMILES: Cc1cn2cc(NC(=O)c3ccc(N4CCNCC4)c4nonc34)cc(F)c2n1] |
| 264 | 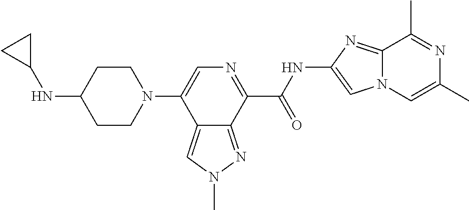 [SMILES: Cc1cn2cc(NC(=O)c3ncc(N4CCC(NC5CC5)CC4)c4cn(C)nc34)nc2c(C)n1] |
| 265 | 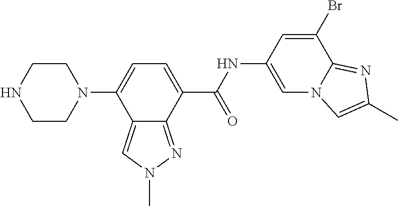 [SMILES: Cc1cn2cc(NC(=O)c3ccc(N4CCNCC4)c4cn(C)nc34)cc(Br)c2n1] |
| 266 | 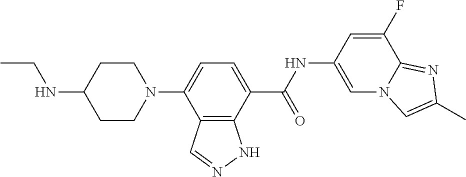 [SMILES: CCNC1CCN(c2ccc(C(=O)Nc3cc(F)c4nc(C)cn4c3)c3[nH]ncc23)CC1] |
| 267 | 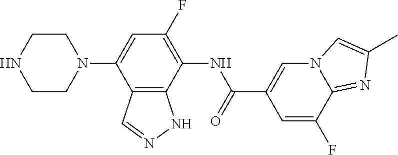 [SMILES: Cc1cn2cc(C(=O)Nc3c(F)cc(N4CCNCC4)c4cn[nH]c34)cc(F)c2n1] |
| 268 | 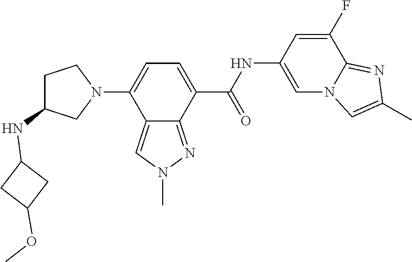 [SMILES: COC1CC(N[C@H]2CCN(c3ccc(C(=O)Nc4cc(F)c5nc(C)cn5c4)c4nn(C)cc34)C2)C1] |
| 269 | 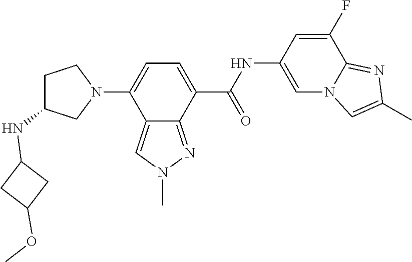 [SMILES: COC1CC(N[C@@H]2CCN(c3ccc(C(=O)Nc4cc(F)c5nc(C)cn5c4)c4nn(C)cc34)C2)C1] |
| 270 | 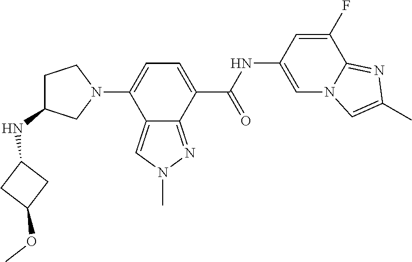 [SMILES: CO[C@H]1C[C@H](N[C@H]2CCN(c3ccc(C(=O)Nc4cc(F)c5nc(C)cn5c4)c4nn(C)cc34)C2)C1] |
| 271 | 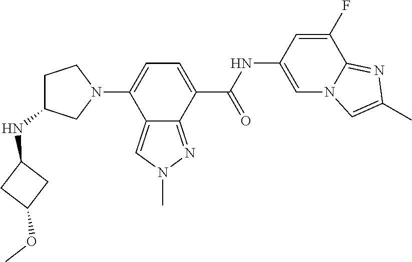 [SMILES: CO[C@H]1C[C@H](N[C@@H]2CCN(c3ccc(C(=O)Nc4cc(F)c5nc(C)cn5c4)c4nn(C)cc34)C2)C1] |
| 272 | 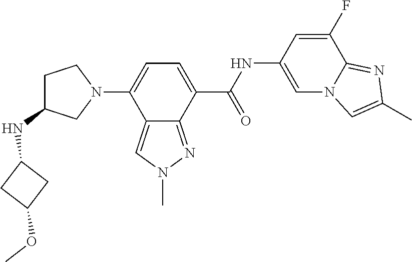 [SMILES: CO[C@H]1C[C@@H](N[C@H]2CCN(c3ccc(C(=O)Nc4cc(F)c5nc(C)cn5c4)c4nn(C)cc34)C2)C1] |
| 273 | 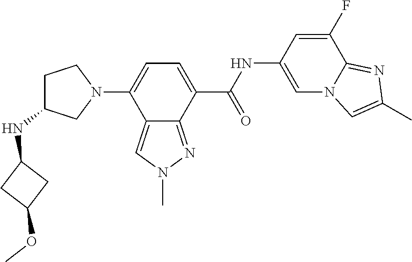 [SMILES: CO[C@H]1C[C@@H](N[C@@H]2CCN(c3ccc(C(=O)Nc4cc(F)c5nc(C)cn5c4)c4nn(C)cc34)C2)C1] |
| 274 | 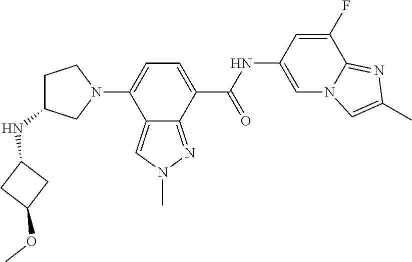 [SMILES: CO[C@H]1C[C@H](N[C@@H]2CCN(c3ccc(C(=O)Nc4cc(F)c5nc(C)cn5c4)c4nn(C)cc34)C2)C1] |
| 275 | 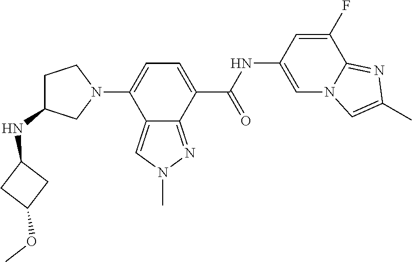 [SMILES: CO[C@H]1C[C@H](N[C@H]2CCN(c3ccc(C(=O)Nc4cc(F)c5nc(C)cn5c4)c4nn(C)cc34)C2)C1] |
| 276 | 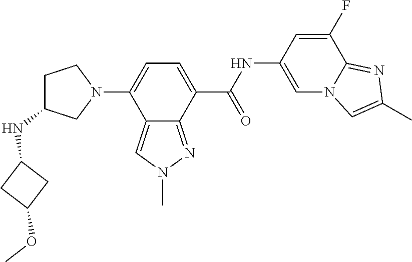 [SMILES: CO[C@H]1C[C@@H](N[C@@H]2CCN(c3ccc(C(=O)Nc4cc(F)c5nc(C)cn5c4)c4nn(C)cc34)C2)C1] |
| 277 | 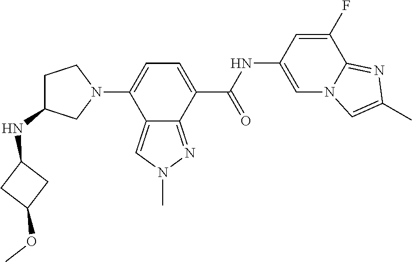 [SMILES: CO[C@H]1C[C@@H](N[C@H]2CCN(c3ccc(C(=O)Nc4cc(F)c5nc(C)cn5c4)c4nn(C)cc34)C2)C1] |
| 278 | 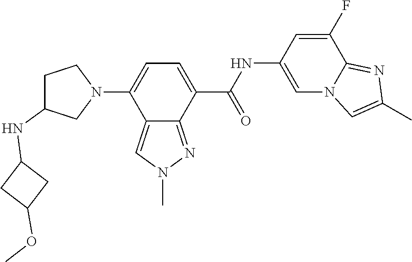 [SMILES: COC1CC(NC2CCN(c3ccc(C(=O)Nc4cc(F)c5nc(C)cn5c4)c4nn(C)cc34)C2)C1] |
| 279 | 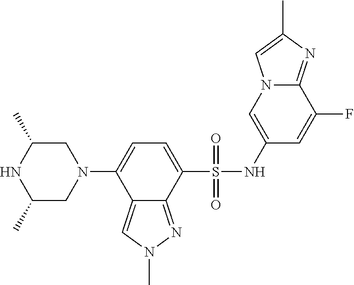 [SMILES: Cc1cn2cc(NS(=O)(=O)c3ccc(N4C[C@@H](C)N[C@@H](C)C4)c4cn(C)nc34)cc(F)c2n1] |
| 280 | 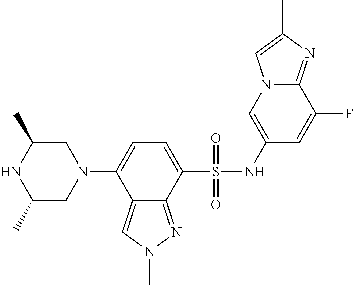 [SMILES: Cc1cn2cc(NS(=O)(=O)c3ccc(N4C[C@H](C)N[C@@H](C)C4)c4cn(C)nc34)cc(F)c2n1] |
| 281 | 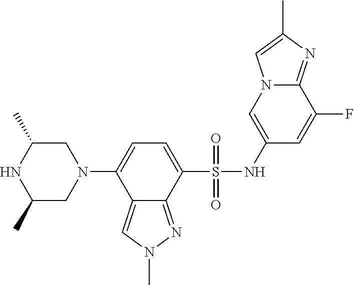 [SMILES: Cc1cn2cc(NS(=O)(=O)c3ccc(N4C[C@@H](C)N[C@H](C)C4)c4cn(C)nc34)cc(F)c2n1] |
| 282 | 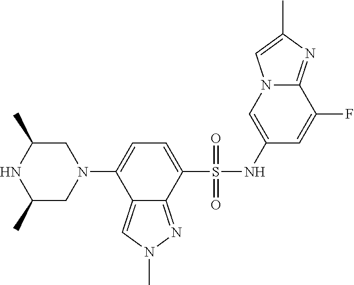 [SMILES: Cc1cn2cc(NS(=O)(=O)c3ccc(N4C[C@@H](C)N[C@@H](C)C4)c4cn(C)nc34)cc(F)c2n1] |
| 283 | 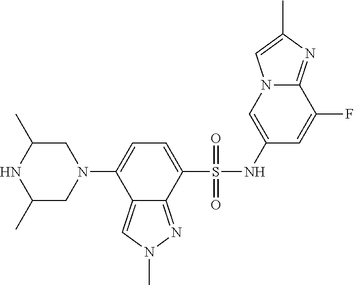 [SMILES: Cc1cn2cc(NS(=O)(=O)c3ccc(N4CC(C)NC(C)C4)c4cn(C)nc34)cc(F)c2n1] |
| 284 | 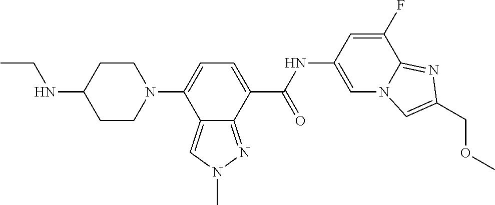 [SMILES: CCNC1CCN(c2ccc(C(=O)Nc3cc(F)c4nc(COC)cn4c3)c3nn(C)cc23)CC1] |
| 285 | 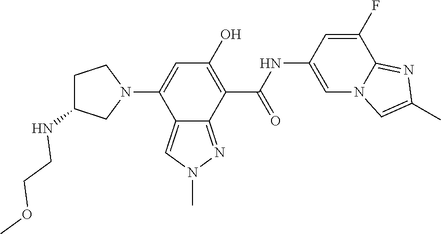 |
| 286 | 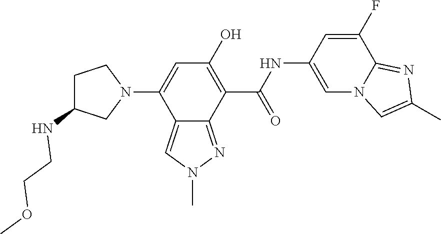 |
| 287 | 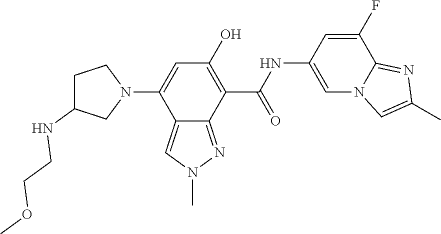 |
| 288 | 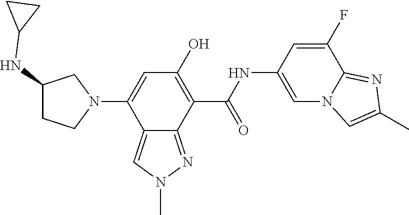 [SMILES: Cc1cn2cc(NC(=O)c3c(O)cc(N4CC[C@@H](NC5CC5)C4)c4cn(C)nc34)cc(F)c2n1] |
| 289 | 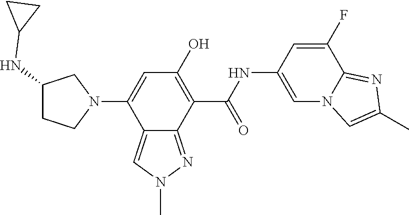 [SMILES: Cc1cn2cc(NC(=O)c3c(O)cc(N4CC[C@H](NC5CC5)C4)c4cn(C)nc34)cc(F)c2n1] |
| 290 | 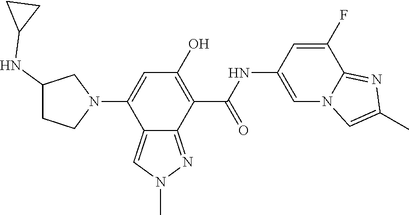 [SMILES: Cc1cn2cc(NC(=O)c3c(O)cc(N4CCC(NC5CC5)C4)c4cn(C)nc34)cc(F)c2n1] |
| 291 | 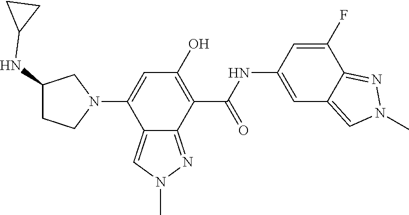 [SMILES: Cn1cc2cc(NC(=O)c3c(O)cc(N4CC[C@@H](NC5CC5)C4)c4cn(C)nc34)cc(F)c2n1] |
| 292 | 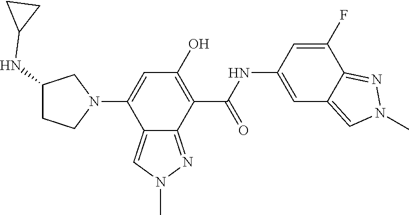 [SMILES: Cn1cc2cc(NC(=O)c3c(O)cc(N4CC[C@H](NC5CC5)C4)c4cn(C)nc34)cc(F)c2n1] |
| 293 | 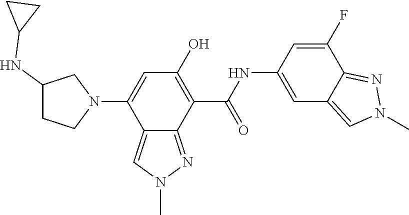 [SMILES: Cn1cc2cc(NC(=O)c3c(O)cc(N4CCC(NC5CC5)C4)c4cn(C)nc34)cc(F)c2n1] |
| 294 | 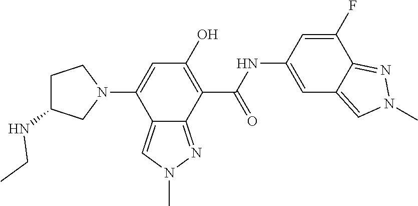 [SMILES: CCN[C@@H]1CCN(c2cc(O)c(C(=O)Nc3cc(F)c4nn(C)cc4c3)c3nn(C)cc23)C1] |
| 295 | 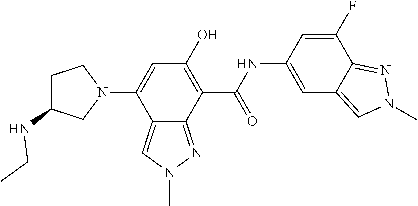 [SMILES: CCN[C@H]1CCN(c2cc(O)c(C(=O)Nc3cc(F)c4nn(C)cc4c3)c3nn(C)cc23)C1] |
| 296 | 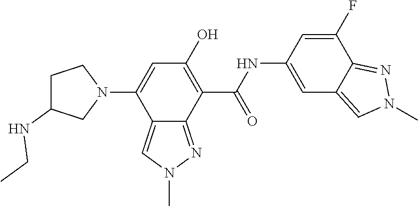 [SMILES: CCNC1CCN(c2cc(O)c(C(=O)Nc3cc(F)c4nn(C)cc4c3)c3nn(C)cc23)C1] |
| 297 | 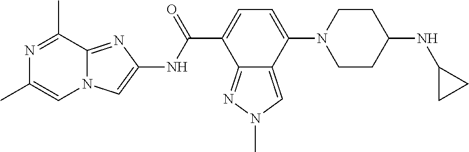 [SMILES: Cc1cn2cc(NC(=O)c3ccc(N4CCC(NC5CC5)CC4)c4cn(C)nc34)nc2c(C)n1] |
| 298 | 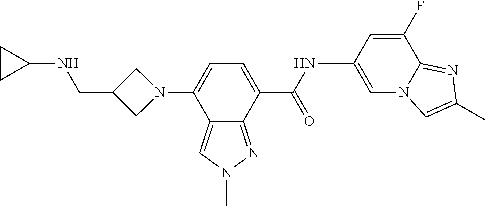 [SMILES: Cc1cn2cc(NC(=O)c3ccc(N4CC(CNC5CC5)C4)c4cn(C)nc34)cc(F)c2n1] |
| 299 | 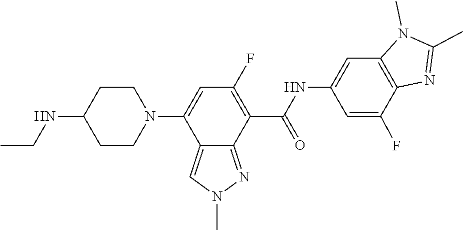 [SMILES: CCNC1CCN(c2cc(F)c(C(=O)Nc3cc(F)c4nc(C)n(C)c4c3)c3nn(C)cc23)CC1] |
| 300 | 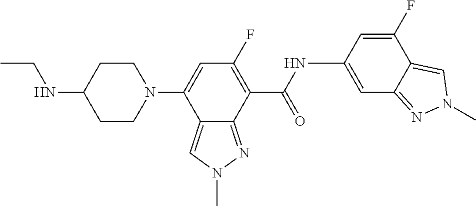 [SMILES: CCNC1CCN(c2cc(F)c(C(=O)Nc3cc(F)c4cn(C)nc4c3)c3nn(C)cc23)CC1] |
| 301 | 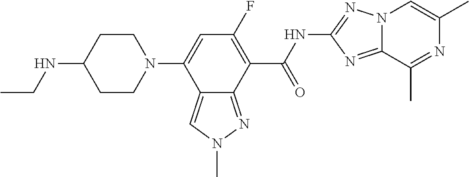 [SMILES: CCNC1CCN(c2cc(F)c(C(=O)Nc3nc4c(C)nc(C)cn4n3)c3nn(C)cc23)CC1] |
| 302 | 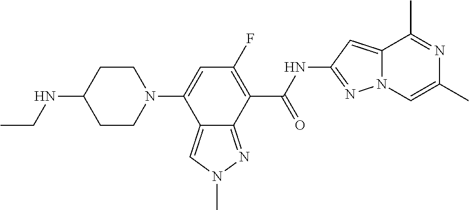 [SMILES: CCNC1CCN(c2cc(F)c(C(=O)Nc3cc4c(C)nc(C)cn4n3)c3nn(C)cc23)CC1] |
| 303 | 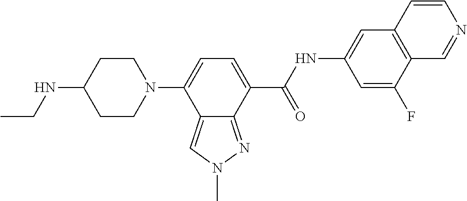 [SMILES: CCNC1CCN(c2ccc(C(=O)Nc3cc(F)c4cnccc4c3)c3nn(C)cc23)CC1] |
| 304 | 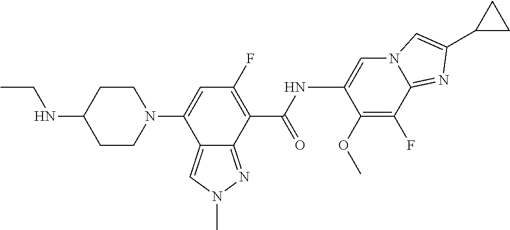 [SMILES: CCNC1CCN(c2cc(F)c(C(=O)Nc3cn4cc(C5CC5)nc4c(F)c3OC)c3nn(C)cc23)CC1] |
| 305 | 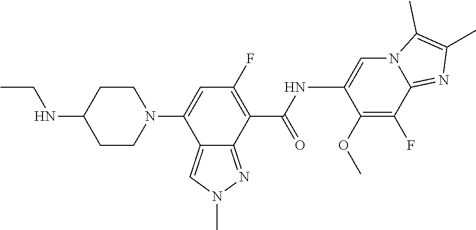 [SMILES: CCNC1CCN(c2cc(F)c(C(=O)Nc3cn4c(C)c(C)nc4c(F)c3OC)c3nn(C)cc23)CC1] |
| 306 | 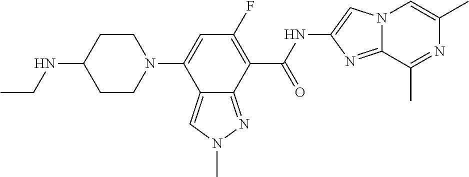 [SMILES: CCNC1CCN(c2cc(F)c(C(=O)Nc3cn4cc(C)nc(C)c4n3)c3nn(C)cc23)CC1] |
| 307 | 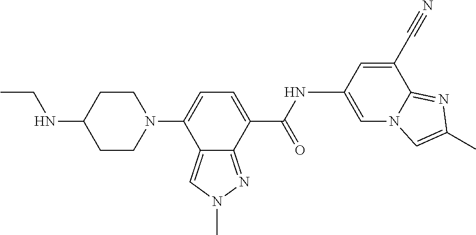 [SMILES: CCNC1CCN(c2ccc(C(=O)Nc3cc(C#N)c4nc(C)cn4c3)c3nn(C)cc23)CC1] |
| 308 | 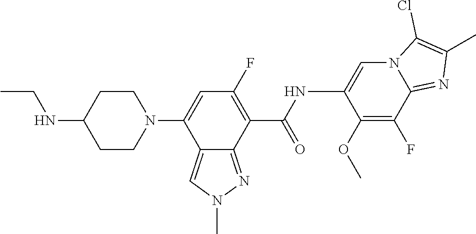 [SMILES: CCNC1CCN(c2cc(F)c(C(=O)Nc3cn4c(Cl)c(C)nc4c(F)c3OC)c3nn(C)cc23)CC1] |
| 309 | 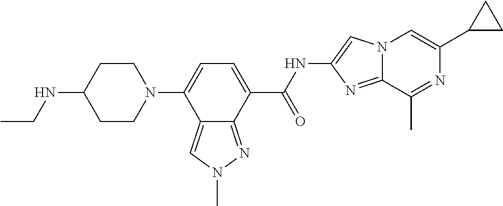 [SMILES: CCNC1CCN(c2ccc(C(=O)Nc3cn4cc(C5CC5)nc(C)c4n3)c3nn(C)cc23)CC1] |
| 310 | 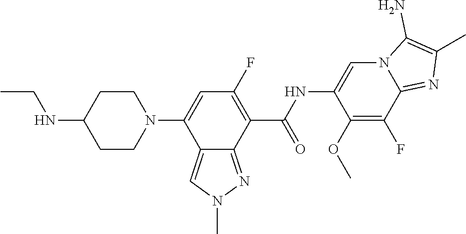 [SMILES: CCNC1CCN(c2cc(F)c(C(=O)Nc3cn4c(N)c(C)nc4c(F)c3OC)c3nn(C)cc23)CC1] |
| 311 | 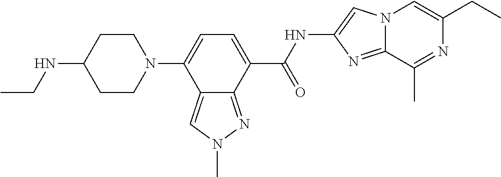 [SMILES: CCNC1CCN(c2ccc(C(=O)Nc3cn4cc(CC)nc(C)c4n3)c3nn(C)cc23)CC1] |
| 312 | 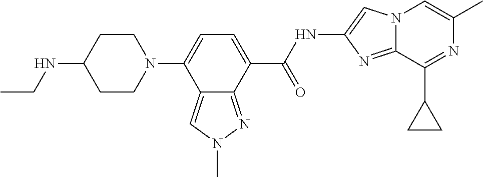 [SMILES: CCNC1CCN(c2ccc(C(=O)Nc3cn4cc(C)nc(C5CC5)c4n3)c3nn(C)cc23)CC1] |
| 313 | 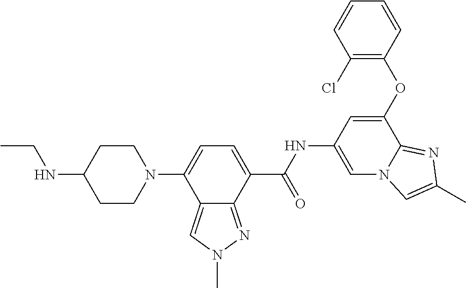 [SMILES: CCNC1CCN(c2ccc(C(=O)Nc3cc(Oc4ccccc4Cl)c4nc(C)cn4c3)c3nn(C)cc23)CC1] |
| 314 | 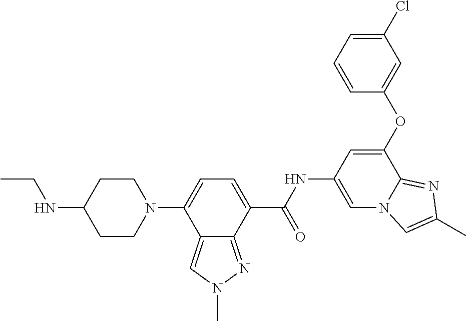 [SMILES: CCNC1CCN(c2ccc(C(=O)Nc3cc(Oc4cccc(Cl)c4)c4nc(C)cn4c3)c3nn(C)cc23)CC1] |
| 315 | 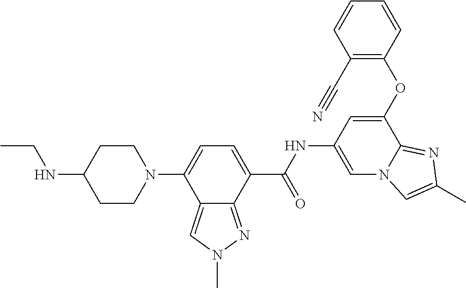 [SMILES: CCNC1CCN(c2ccc(C(=O)Nc3cc(Oc4ccccc4C#N)c4nc(C)cn4c3)c3nn(C)cc23)CC1] |
| 316 | 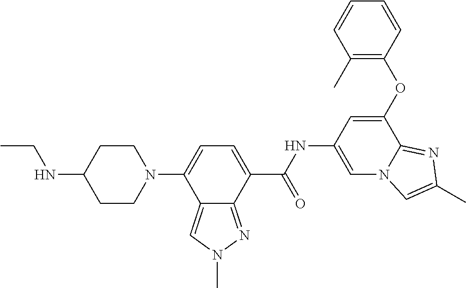 [SMILES: CCNC1CCN(c2ccc(C(=O)Nc3cc(Oc4ccccc4C)c4nc(C)cn4c3)c3nn(C)cc23)CC1] |
| 317 | 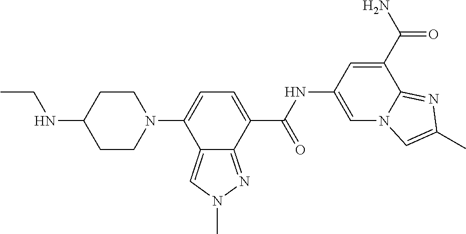 [SMILES: CCNC1CCN(c2ccc(C(=O)Nc3cc(C(N)=O)c4nc(C)cn4c3)c3nn(C)cc23)CC1] |
| 318 | 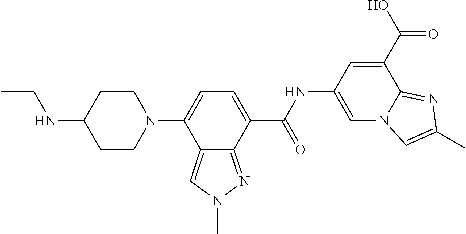 [SMILES: CCNC1CCN(c2ccc(C(=O)Nc3cc(C(=O)O)c4nc(C)cn4c3)c3nn(C)cc23)CC1] |
| 319 | 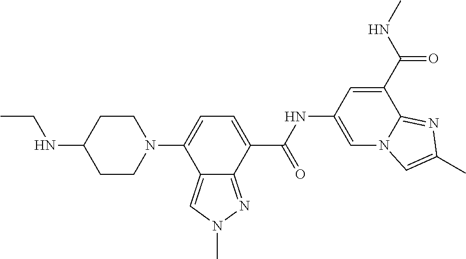 [SMILES: CCNC1CCN(c2ccc(C(=O)Nc3cc(C(=O)NC)c4nc(C)cn4c3)c3nn(C)cc23)CC1] |
| 320 | 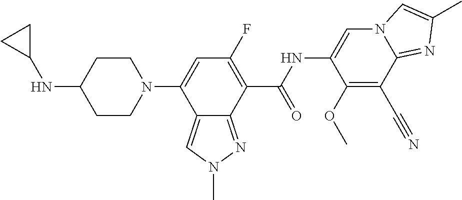 [SMILES: COc1c(NC(=O)c2c(F)cc(N3CCC(NC4CC4)CC3)c3cn(C)nc23)cn2cc(C)nc2c1C#N] |
| 321 | 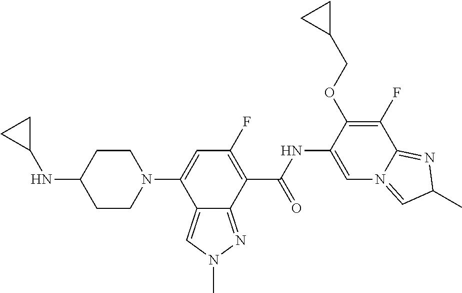 |
| 322 | 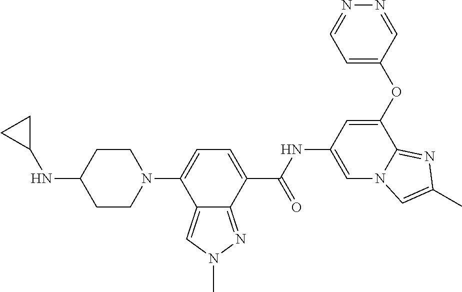 [SMILES: Cc1cn2cc(NC(=O)c3ccc(N4CCC(NC5CC5)CC4)c4cn(C)nc34)cc(Oc3ccnnc3)c2n1] |
| 323 | 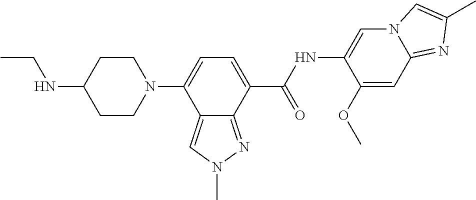 [SMILES: CCNC1CCN(c2ccc(C(=O)Nc3cn4cc(C)nc4cc3OC)c3nn(C)cc23)CC1] |
| 324 | 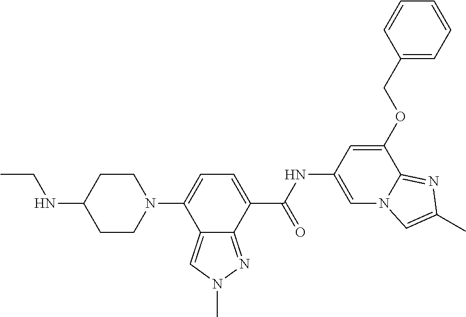 [SMILES: CCNC1CCN(c2ccc(C(=O)Nc3cc(OCc4ccccc4)c4nc(C)cn4c3)c3nn(C)cc23)CC1] |
| 325 | 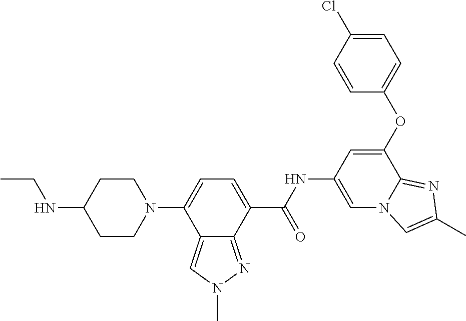 [SMILES: CCNC1CCN(c2ccc(C(=O)Nc3cc(Oc4ccc(Cl)cc4)c4nc(C)cn4c3)c3nn(C)cc23)CC1] |
| 326 | 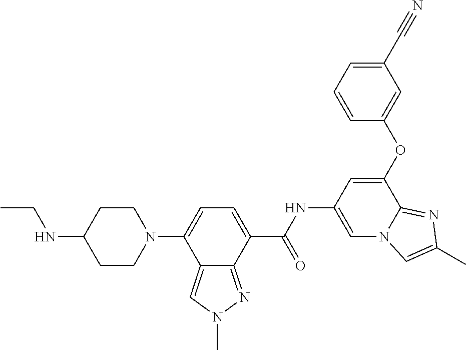 [SMILES: CCNC1CCN(c2ccc(C(=O)Nc3cc(Oc4cccc(C#N)c4)c4nc(C)cn4c3)c3nn(C)cc23)CC1] |
| 327 | 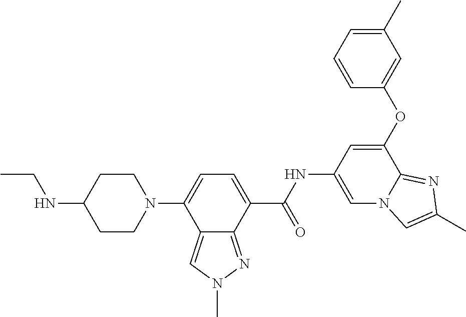 [SMILES: CCNC1CCN(c2ccc(C(=O)Nc3cc(Oc4cccc(C)c4)c4nc(C)cn4c3)c3nn(C)cc23)CC1] |
| 328 | 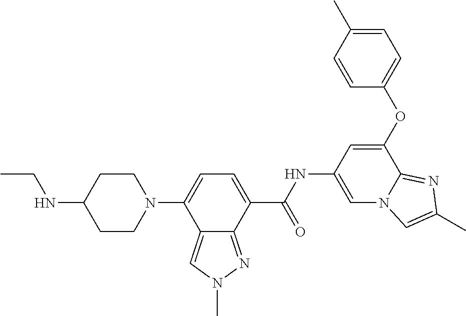 [SMILES: CCNC1CCN(c2ccc(C(=O)Nc3cc(Oc4ccc(C)cc4)c4nc(C)cn4c3)c3nn(C)cc23)CC1] |
| 329 | 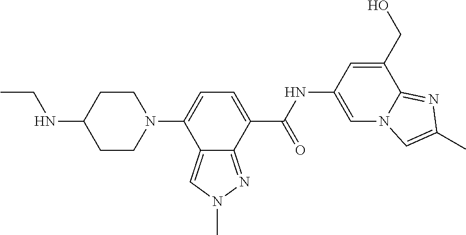 [SMILES: CCNC1CCN(c2ccc(C(=O)Nc3cc(CO)c4nc(C)cn4c3)c3nn(C)cc23)CC1] |
| 330 | 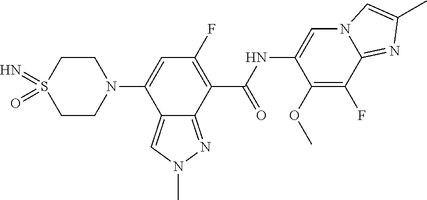 [SMILES: COc1c(NC(=O)c2c(F)cc(N3CCS(=N)(=O)CC3)c3cn(C)nc23)cn2cc(C)nc2c1F] |
| 331 | 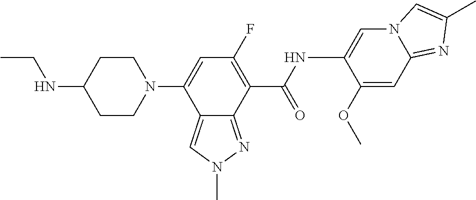 [SMILES: CCNC1CCN(c2cc(F)c(C(=O)Nc3cn4cc(C)nc4cc3OC)c3nn(C)cc23)CC1] |
| 332 | 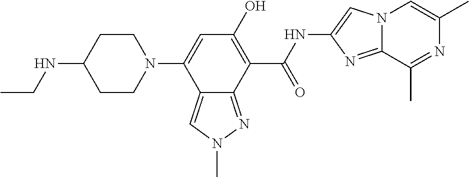 [SMILES: CCNC1CCN(c2cc(O)c(C(=O)Nc3cn4cc(C)nc(C)c4n3)c3nn(C)cc23)CC1] |
| 333 | 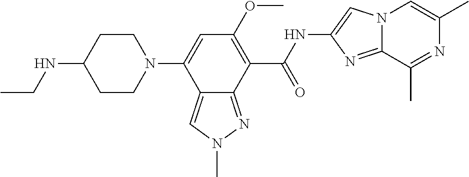 [SMILES: CCNC1CCN(c2cc(OC)c(C(=O)Nc3cn4cc(C)nc(C)c4n3)c3nn(C)cc23)CC1] |
| 334 | 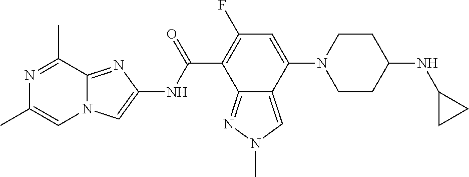 [SMILES: Cc1cn2cc(NC(=O)c3c(F)cc(N4CCC(NC5CC5)CC4)c4cn(C)nc34)nc2c(C)n1] |
| 335 | 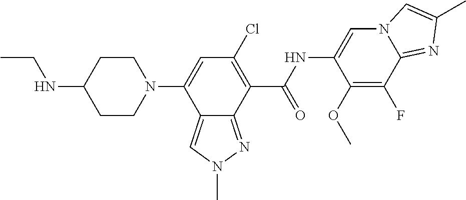 [SMILES: CCNC1CCN(c2cc(Cl)c(C(=O)Nc3cn4cc(C)nc4c(F)c3OC)c3nn(C)cc23)CC1] |
| 336 | 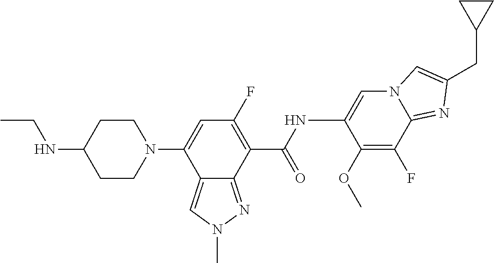 [SMILES: CCNC1CCN(c2cc(F)c(C(=O)Nc3cn4cc(CC5CC5)nc4c(F)c3OC)c3nn(C)cc23)CC1] |
| 337 | 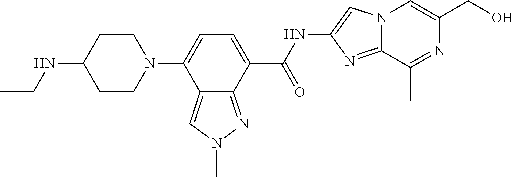 [SMILES: CCNC1CCN(c2ccc(C(=O)Nc3cn4cc(CO)nc(C)c4n3)c3nn(C)cc23)CC1] |
| 338 | 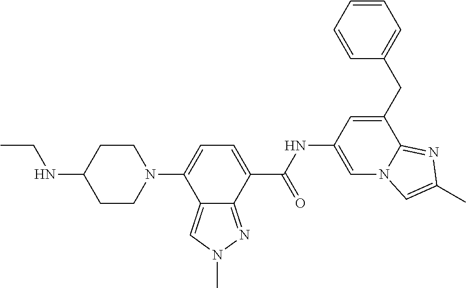 [SMILES: CCNC1CCN(c2ccc(C(=O)Nc3cc(Cc4ccccc4)c4nc(C)cn4c3)c3nn(C)cc23)CC1] |
| 339 | 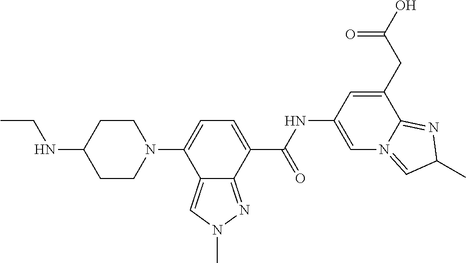 |
| 340 | 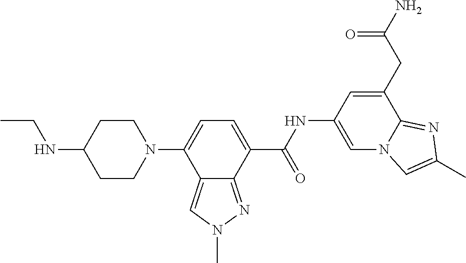 [SMILES: CCNC1CCN(c2ccc(C(=O)Nc3cc(CC(N)=O)c4nc(C)cn4c3)c3nn(C)cc23)CC1] |
| 341 | 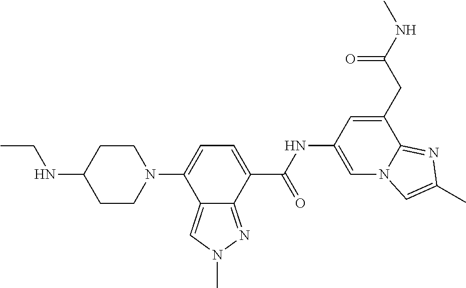 [SMILES: CCNC1CCN(c2ccc(C(=O)Nc3cc(CC(=O)NC)c4nc(C)cn4c3)c3nn(C)cc23)CC1] |
| 342 | 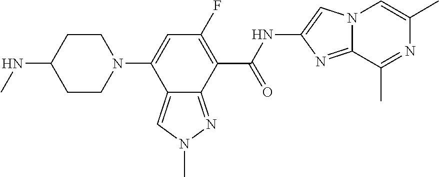 |
| 343 | 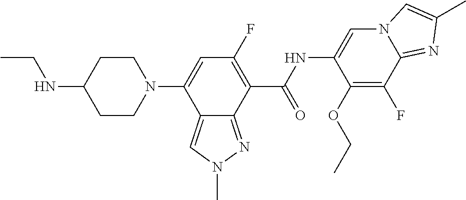 [SMILES: CCNC1CCN(c2cc(F)c(C(=O)Nc3cn4cc(C)nc4c(F)c3OCC)c3nn(C)cc23)CC1] |
| 344 | 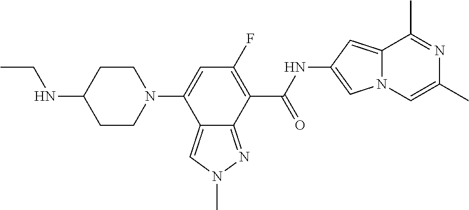 [SMILES: CCNC1CCN(c2cc(F)c(C(=O)Nc3cc4c(C)nc(C)cn4c3)c3nn(C)cc23)CC1] |
| 345 | 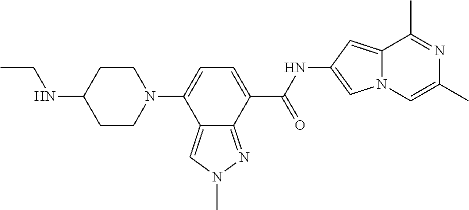 [SMILES: CCNC1CCN(c2ccc(C(=O)Nc3cc4c(C)nc(C)cn4c3)c3nn(C)cc23)CC1] |
| 346 | 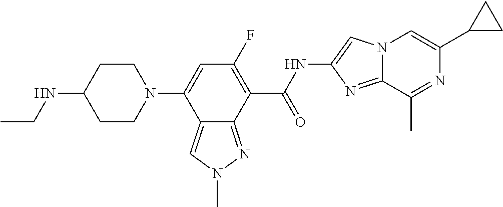 [SMILES: CCNC1CCN(c2cc(F)c(C(=O)Nc3cn4cc(C5CC5)nc(C)c4n3)c3nn(C)cc23)CC1] |
| 347 | 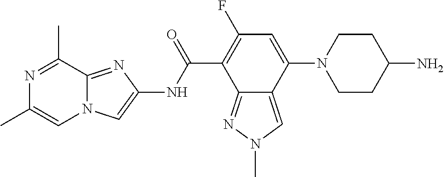 [SMILES: Cc1cn2cc(NC(=O)c3c(F)cc(N4CCC(N)CC4)c4cn(C)nc34)nc2c(C)n1] |
| 348 | 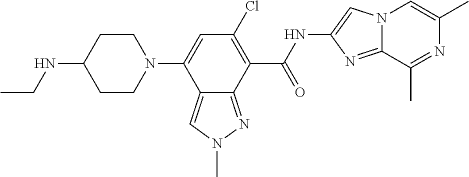 [SMILES: CCNC1CCN(c2cc(Cl)c(C(=O)Nc3cn4cc(C)nc(C)c4n3)c3nn(C)cc23)CC1] |
| 349 | 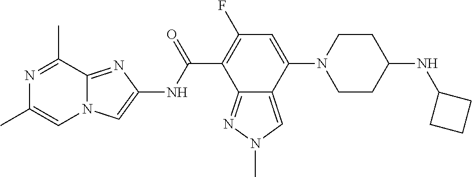 [SMILES: Cc1cn2cc(NC(=O)c3c(F)cc(N4CCC(NC5CCC5)CC4)c4cn(C)nc34)nc2c(C)n1] |
| 350 | 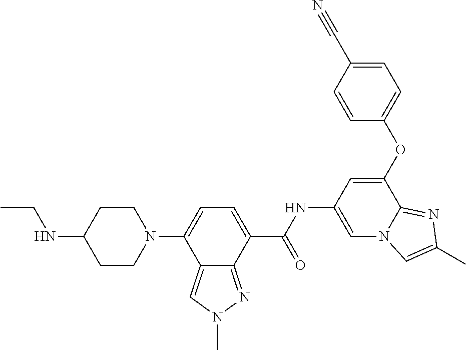 [SMILES: CCNC1CCN(c2ccc(C(=O)Nc3cc(Oc4ccc(C#N)cc4)c4nc(C)cn4c3)c3nn(C)cc23)CC1] |
| 351 | 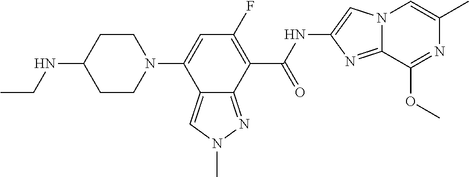 [SMILES: CCNC1CCN(c2cc(F)c(C(=O)Nc3cn4cc(C)nc(OC)c4n3)c3nn(C)cc23)CC1] |
| 352 | 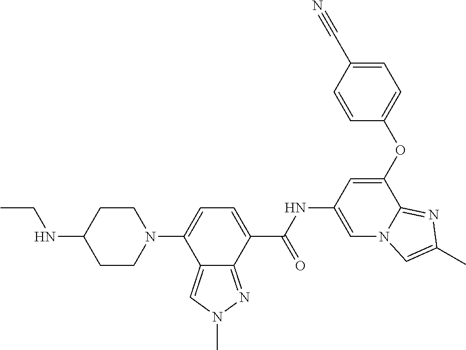 [SMILES: CCNC1CCN(c2ccc(C(=O)Nc3cc(Oc4ccc(C#N)cc4)c4nc(C)cn4c3)c3nn(C)cc23)CC1] |
| 353 | 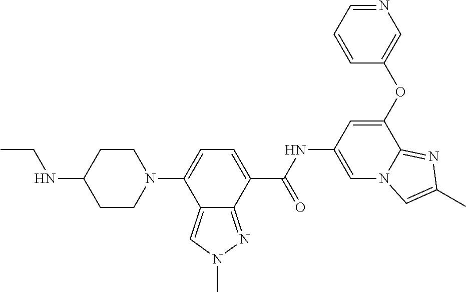 [SMILES: CCNC1CCN(c2ccc(C(=O)Nc3cc(Oc4cccnc4)c4nc(C)cn4c3)c3nn(C)cc23)CC1] |
| 354 | 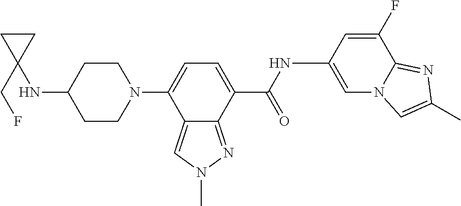 [SMILES: Cc1cn2cc(NC(=O)c3ccc(N4CCC(NC5(CF)CC5)CC4)c4cn(C)nc34)cc(F)c2n1] |
| 355 | 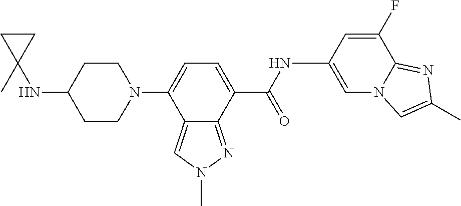 |
| 356 | 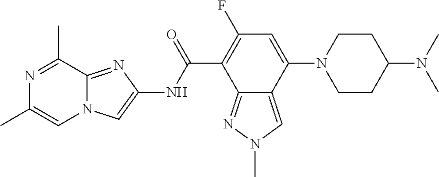 |
| 357 | 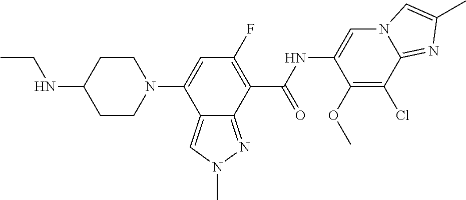 [SMILES: CCNC1CCN(c2cc(F)c(C(=O)Nc3cn4cc(C)nc4c(Cl)c3OC)c3nn(C)cc23)CC1] |
| 358 | 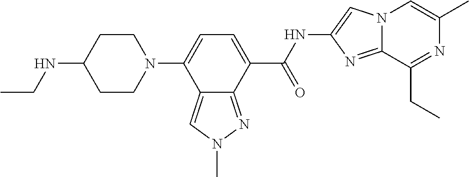 [SMILES: CCNC1CCN(c2ccc(C(=O)Nc3cn4cc(C)nc(CC)c4n3)c3nn(C)cc23)CC1] |
| 359 | 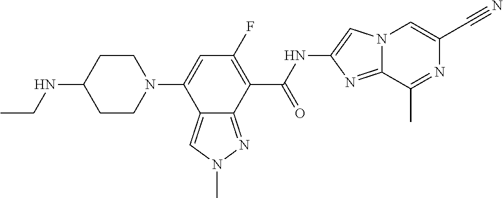 [SMILES: CCNC1CCN(c2cc(F)c(C(=O)Nc3cn4cc(C#N)nc(C)c4n3)c3nn(C)cc23)CC1] |
| 360 | 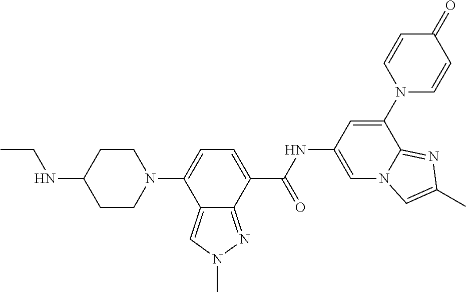 [SMILES: CCNC1CCN(c2ccc(C(=O)Nc3cc(-n4ccc(=O)cc4)c4nc(C)cn4c3)c3nn(C)cc23)CC1] |
| 361 | 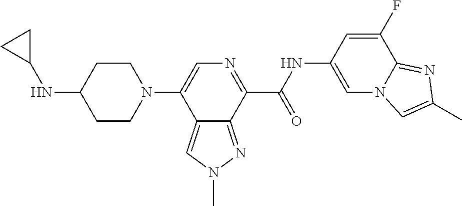 [SMILES: Cc1cn2cc(NC(=O)c3ncc(N4CCC(NC5CC5)CC4)c4cn(C)nc34)cc(F)c2n1] |
| 362 | 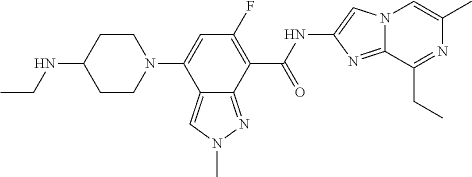 [SMILES: CCNC1CCN(c2cc(F)c(C(=O)Nc3cn4cc(C)nc(CC)c4n3)c3nn(C)cc23)CC1] |
| 363 | 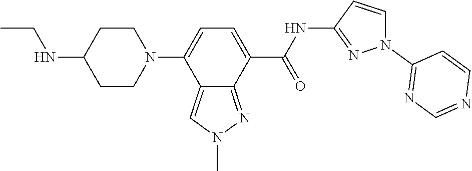 [SMILES: CCNC1CCN(c2ccc(C(=O)Nc3ccn(-c4ccncn4)n3)c3nn(C)cc23)CC1] |
| 364 | 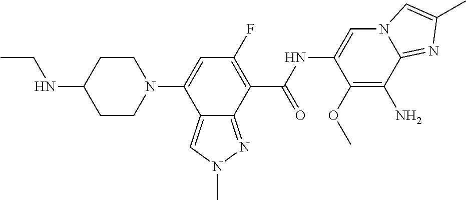 [SMILES: CCNC1CCN(c2cc(F)c(C(=O)Nc3cn4cc(C)nc4c(N)c3OC)c3nn(C)cc23)CC1] |
| 365 | 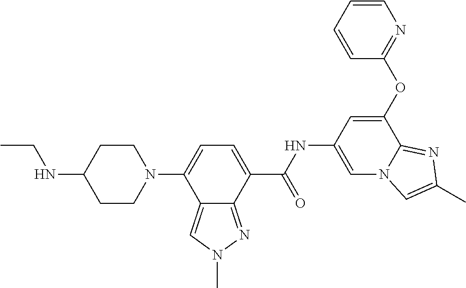 [SMILES: CCNC1CCN(c2ccc(C(=O)Nc3cc(Oc4ccccn4)c4nc(C)cn4c3)c3nn(C)cc23)CC1] |
| 366 | 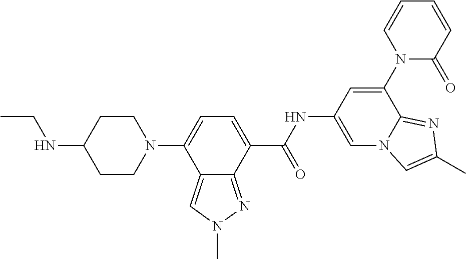 [SMILES: CCNC1CCN(c2ccc(C(=O)Nc3cc(-n4ccccc4=O)c4nc(C)cn4c3)c3nn(C)cc23)CC1] |
| 367 | 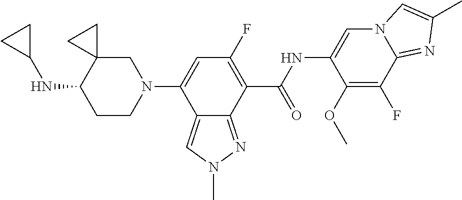 [SMILES: COc1c(NC(=O)c2c(F)cc(N3CC[C@H](NC4CC4)C4(CC4)C3)c3cn(C)nc23)cn2cc(C)nc2c1F] |
| 368 | 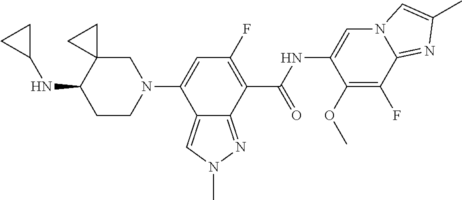 [SMILES: COc1c(NC(=O)c2c(F)cc(N3CC[C@@H](NC4CC4)C4(CC4)C3)c3cn(C)nc23)cn2cc(C)nc2c1F] |
| 369 | 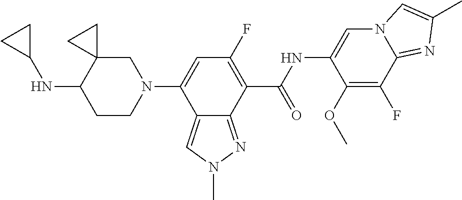 [SMILES: COc1c(NC(=O)c2c(F)cc(N3CCC(NC4CC4)C4(CC4)C3)c3cn(C)nc23)cn2cc(C)nc2c1F] |
| 370 | 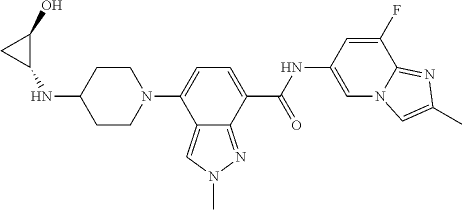 [SMILES: Cc1cn2cc(NC(=O)c3ccc(N4CCC(N[C@@H]5C[C@H]5O)CC4)c4cn(C)nc34)cc(F)c2n1] |
| 371 | 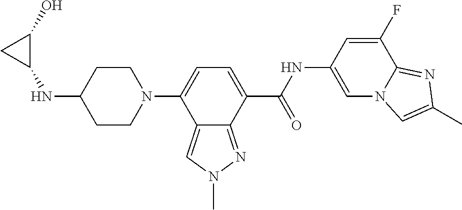 [SMILES: Cc1cn2cc(NC(=O)c3ccc(N4CCC(N[C@@H]5C[C@@H]5O)CC4)c4cn(C)nc34)cc(F)c2n1] |
| 372 | 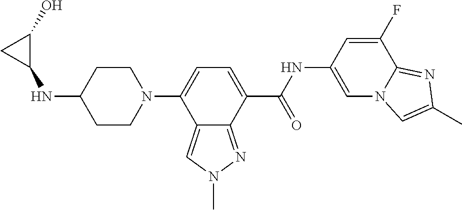 [SMILES: Cc1cn2cc(NC(=O)c3ccc(N4CCC(N[C@H]5C[C@@H]5O)CC4)c4cn(C)nc34)cc(F)c2n1] |
| 373 | 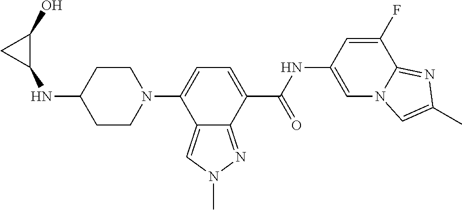 [SMILES: Cc1cn2cc(NC(=O)c3ccc(N4CCC(N[C@H]5C[C@H]5O)CC4)c4cn(C)nc34)cc(F)c2n1] |
| 374 | 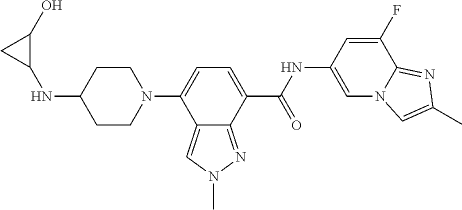 [SMILES: Cc1cn2cc(NC(=O)c3ccc(N4CCC(NC5CC5O)CC4)c4cn(C)nc34)cc(F)c2n1] |
| 375 | 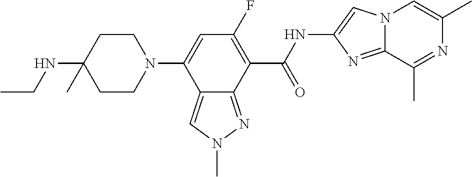 [SMILES: CCNC1(C)CCN(c2cc(F)c(C(=O)Nc3cn4cc(C)nc(C)c4n3)c3nn(C)cc23)CC1] |
| 376 | 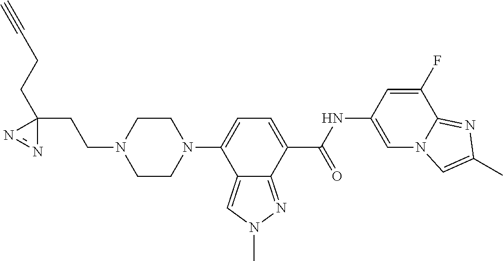 [SMILES: C#CCCC1(CCN2CCN(c3ccc(C(=O)Nc4cc(F)c5nc(C)cn5c4)c4nn(C)cc34)CC2)N=N1] |
| 377 | 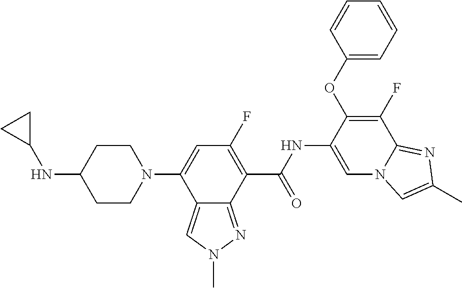 [SMILES: Cc1cn2cc(NC(=O)c3c(F)cc(N4CCC(NC5CC5)CC4)c4cn(C)nc34)c(Oc3ccccc3)c(F)c2n1] |
| 378 | 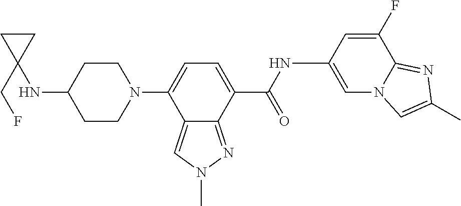 [SMILES: Cc1cn2cc(NC(=O)c3ccc(N4CCC(NC5(CF)CC5)CC4)c4cn(C)nc34)cc(F)c2n1] |
| 379 | 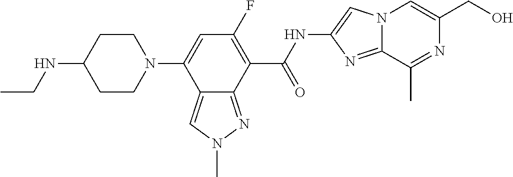 [SMILES: CCNC1CCN(c2cc(F)c(C(=O)Nc3cn4cc(CO)nc(C)c4n3)c3nn(C)cc23)CC1] |
| 380 | 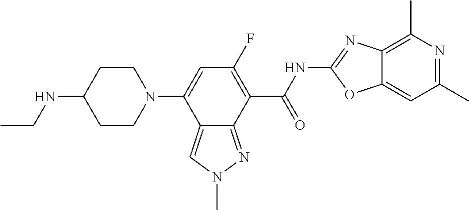 [SMILES: CCNC1CCN(c2cc(F)c(C(=O)Nc3nc4c(C)nc(C)cc4o3)c3nn(C)cc23)CC1] |
| 381 | 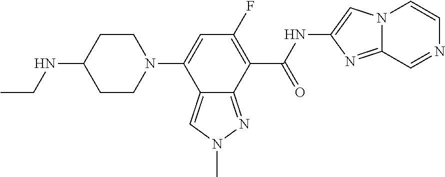 [SMILES: CCNC1CCN(c2cc(F)c(C(=O)Nc3cn4ccncc4n3)c3nn(C)cc23)CC1] |
| 382 | 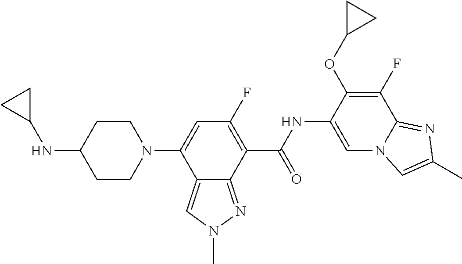 [SMILES: Cc1cn2cc(NC(=O)c3c(F)cc(N4CCC(NC5CC5)CC4)c4cn(C)nc34)c(OC3CC3)c(F)c2n1] |
| 383 | 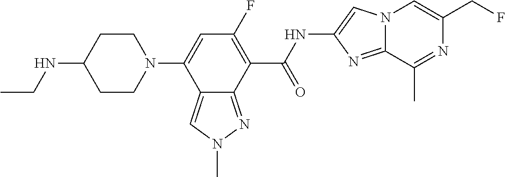 [SMILES: CCNC1CCN(c2cc(F)c(C(=O)Nc3cn4cc(CF)nc(C)c4n3)c3nn(C)cc23)CC1] |
| 384 | 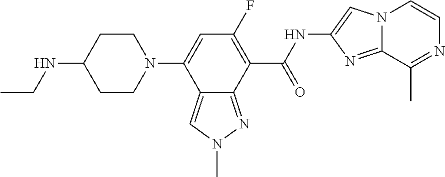 [SMILES: CCNC1CCN(c2cc(F)c(C(=O)Nc3cn4ccnc(C)c4n3)c3nn(C)cc23)CC1] |
| 385 | 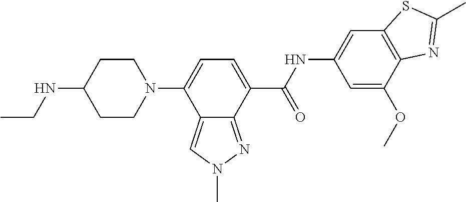 [SMILES: CCNC1CCN(c2ccc(C(=O)Nc3cc(OC)c4nc(C)sc4c3)c3nn(C)cc23)CC1] |
| 386 | 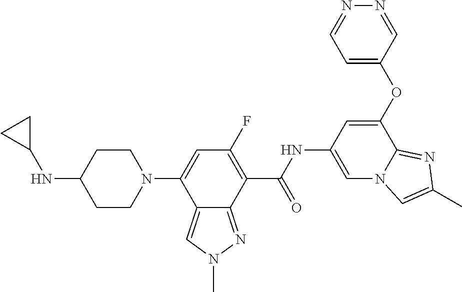 [SMILES: Cc1cn2cc(NC(=O)c3c(F)cc(N4CCC(NC5CC5)CC4)c4cn(C)nc34)cc(Oc3ccnnc3)c2n1] |
| 387 | 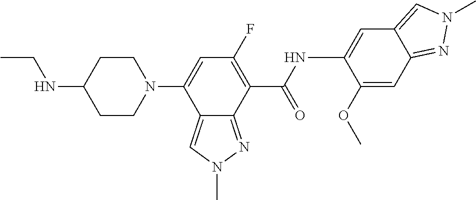 [SMILES: CCNC1CCN(c2cc(F)c(C(=O)Nc3cc4cn(C)nc4cc3OC)c3nn(C)cc23)CC1] |
| 388 | 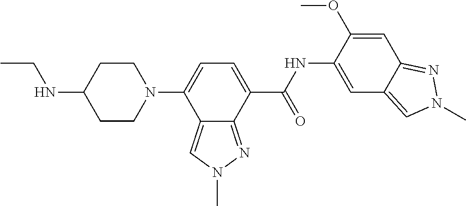 [SMILES: CCNC1CCN(c2ccc(C(=O)Nc3cc4cn(C)nc4cc3OC)c3nn(C)cc23)CC1] |
| 389 | 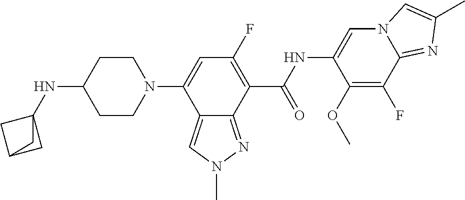 [SMILES: COc1c(NC(=O)c2c(F)cc(N3CCC(NC45CC(C4)C5)CC3)c3cn(C)nc23)cn2cc(C)nc2c1F] |
| 390 | 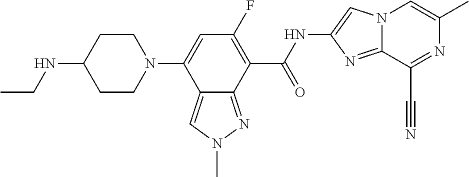 [SMILES: CCNC1CCN(c2cc(F)c(C(=O)Nc3cn4cc(C)nc(C#N)c4n3)c3nn(C)cc23)CC1] |
| 391 | 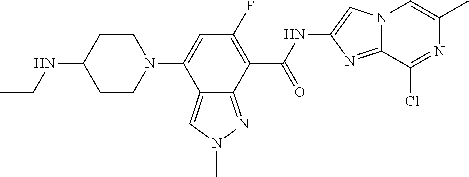 [SMILES: CCNC1CCN(c2cc(F)c(C(=O)Nc3cn4cc(C)nc(Cl)c4n3)c3nn(C)cc23)CC1] |
| 392 | 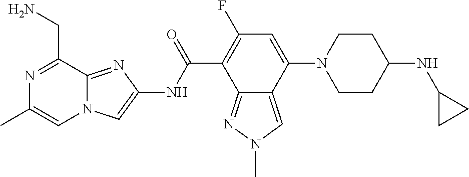 [SMILES: Cc1cn2cc(NC(=O)c3c(F)cc(N4CCC(NC5CC5)CC4)c4cn(C)nc34)nc2c(CN)n1] |
| 393 | 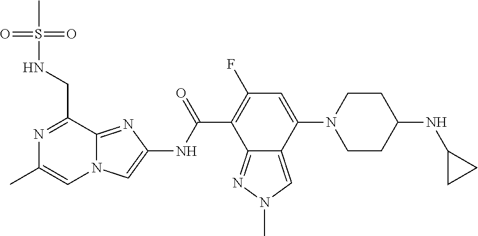 [SMILES: Cc1cn2cc(NC(=O)c3c(F)cc(N4CCC(NC5CC5)CC4)c4cn(C)nc34)nc2c(CNS(C)(=O)=O)n1] |
| 394 | 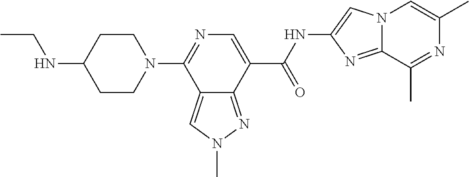 [SMILES: CCNC1CCN(c2ncc(C(=O)Nc3cn4cc(C)nc(C)c4n3)c3nn(C)cc23)CC1] |
| 395 | 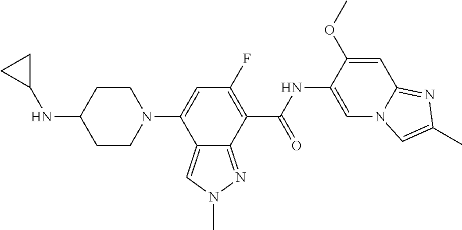 [SMILES: COc1cc2nc(C)cn2cc1NC(=O)c1c(F)cc(N2CCC(NC3CC3)CC2)c2cn(C)nc12] |
| 396 | 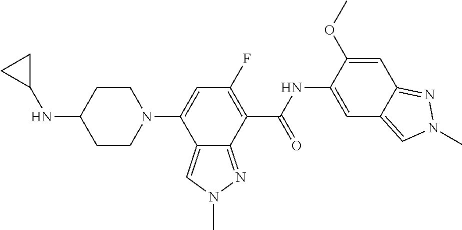 [SMILES: COc1cc2nn(C)cc2cc1NC(=O)c1c(F)cc(N2CCC(NC3CC3)CC2)c2cn(C)nc12] |
| 397 | 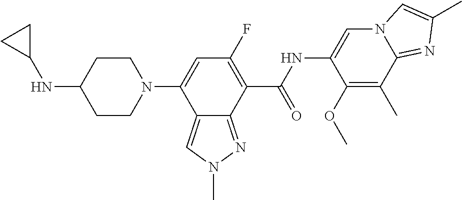 [SMILES: COc1c(NC(=O)c2c(F)cc(N3CCC(NC4CC4)CC3)c3cn(C)nc23)cn2cc(C)nc2c1C] |
| 398 | 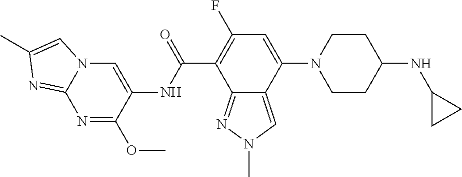 [SMILES: COc1nc2nc(C)cn2cc1NC(=O)c1c(F)cc(N2CCC(NC3CC3)CC2)c2cn(C)nc12] |
| 399 | 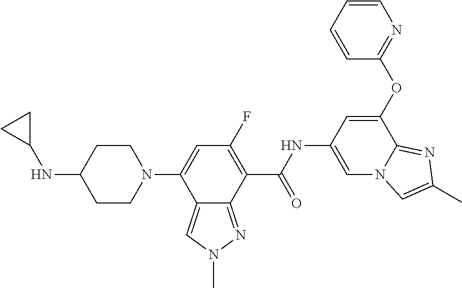 [SMILES: Cc1cn2cc(NC(=O)c3c(F)cc(N4CCC(NC5CC5)CC4)c4cn(C)nc34)cc(Oc3ccccn3)c2n1] |
| 400 | 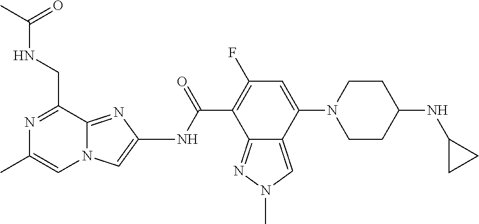 [SMILES: CC(=O)NCc1nc(C)cn2cc(NC(=O)c3c(F)cc(N4CCC(NC5CC5)CC4)c4cn(C)nc34)nc12] |
| 401 | 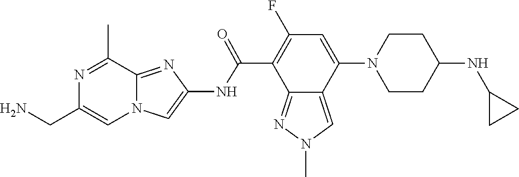 [SMILES: Cc1nc(CN)cn2cc(NC(=O)c3c(F)cc(N4CCC(NC5CC5)CC4)c4cn(C)nc34)nc12] |
| 402 | 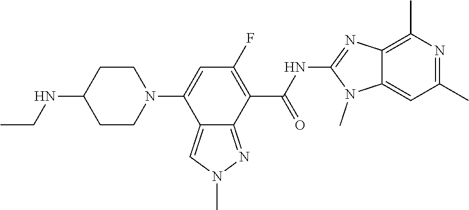 [SMILES: CCNC1CCN(c2cc(F)c(C(=O)Nc3nc4c(C)nc(C)cc4n3C)c3nn(C)cc23)CC1] |
| 403 | 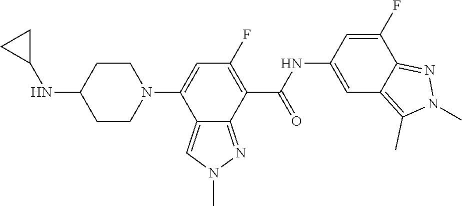 [SMILES: Cc1c2cc(NC(=O)c3c(F)cc(N4CCC(NC5CC5)CC4)c4cn(C)nc34)cc(F)c2nn1C] |
| 404 | 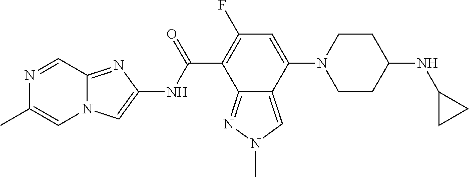 [SMILES: Cc1cn2cc(NC(=O)c3c(F)cc(N4CCC(NC5CC5)CC4)c4cn(C)nc34)nc2cn1] |
| 405 | 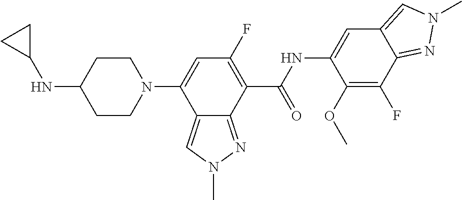 [SMILES: COc1c(NC(=O)c2c(F)cc(N3CCC(NC4CC4)CC3)c3cn(C)nc23)cc2cn(C)nc2c1F] |
| 406 | 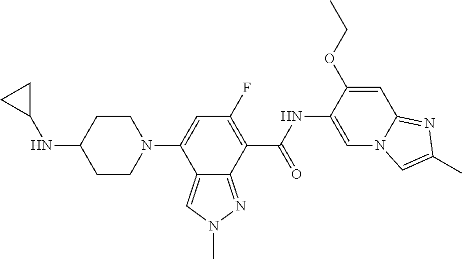 [SMILES: CCOc1cc2nc(C)cn2cc1NC(=O)c1c(F)cc(N2CCC(NC3CC3)CC2)c2cn(C)nc12] |
| 407 | 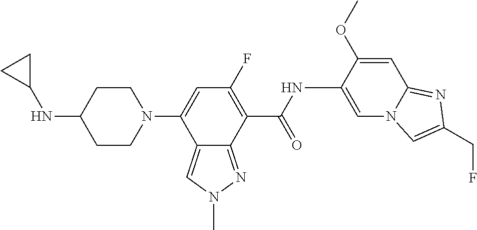 [SMILES: COc1cc2nc(CF)cn2cc1NC(=O)c1c(F)cc(N2CCC(NC3CC3)CC2)c2cn(C)nc12] |
| 408 | 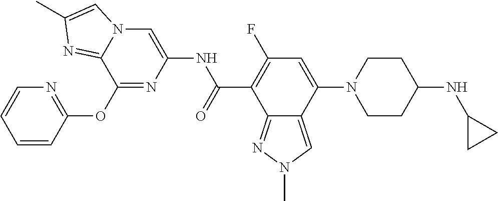 [SMILES: Cc1cn2cc(NC(=O)c3c(F)cc(N4CCC(NC5CC5)CC4)c4cn(C)nc34)nc(Oc3ccccn3)c2n1] |
| 409 | 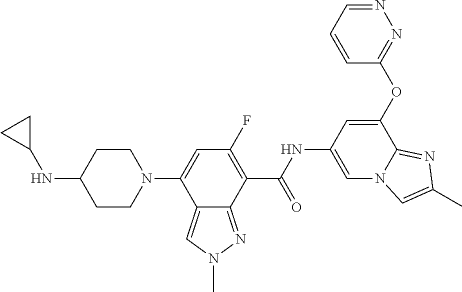 [SMILES: Cc1cn2cc(NC(=O)c3c(F)cc(N4CCC(NC5CC5)CC4)c4cn(C)nc34)cc(Oc3cccnn3)c2n1] |
| 410 | 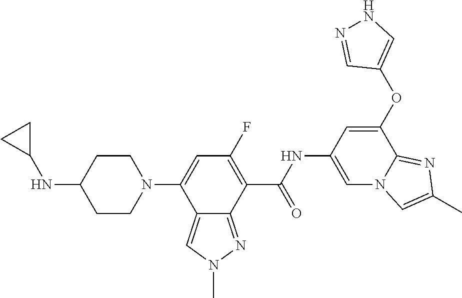 [SMILES: Cc1cn2cc(NC(=O)c3c(F)cc(N4CCC(NC5CC5)CC4)c4cn(C)nc34)cc(Oc3cn[nH]c3)c2n1] |
| 411 | 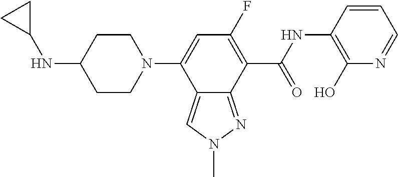 [SMILES: Cn1cc2c(N3CCC(NC4CC4)CC3)cc(F)c(C(=O)Nc3cccnc3O)c2n1] |
| 412 | 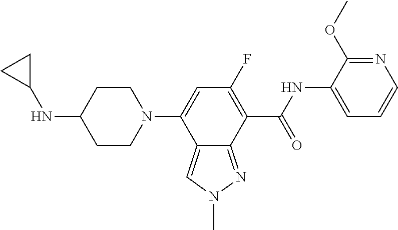 [SMILES: COc1ncccc1NC(=O)c1c(F)cc(N2CCC(NC3CC3)CC2)c2cn(C)nc12] |
| 413 | 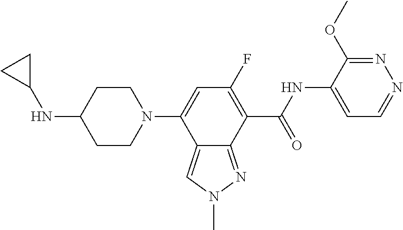 [SMILES: COc1nnccc1NC(=O)c1c(F)cc(N2CCC(NC3CC3)CC2)c2cn(C)nc12] |
| 414 | 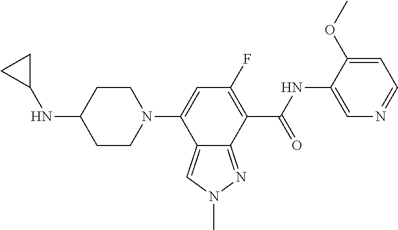 [SMILES: COc1ccncc1NC(=O)c1c(F)cc(N2CCC(NC3CC3)CC2)c2cn(C)nc12] |
| 415 | 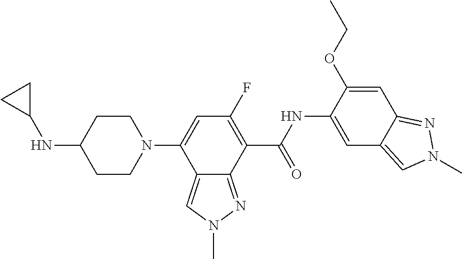 [SMILES: CCOc1cc2nn(C)cc2cc1NC(=O)c1c(F)cc(N2CCC(NC3CC3)CC2)c2cn(C)nc12] |
| 416 | 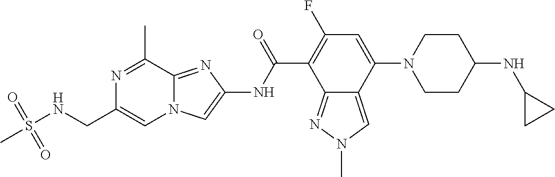 [SMILES: Cc1nc(CNS(C)(=O)=O)cn2cc(NC(=O)c3c(F)cc(N4CCC(NC5CC5)CC4)c4cn(C)nc34)nc12] |
| 417 | 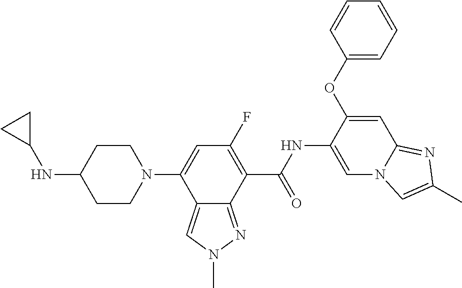 [SMILES: Cc1cn2cc(NC(=O)c3c(F)cc(N4CCC(NC5CC5)CC4)c4cn(C)nc34)c(Oc3ccccc3)cc2n1] |
| 418 | 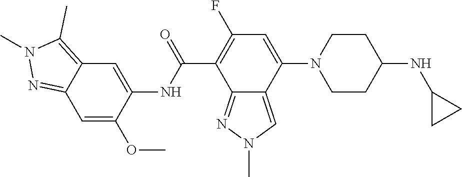 [SMILES: COc1cc2nn(C)c(C)c2cc1NC(=O)c1c(F)cc(N2CCC(NC3CC3)CC2)c2cn(C)nc12] |
| 419 | 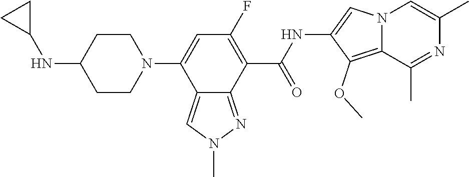 [SMILES: COc1c(NC(=O)c2c(F)cc(N3CCC(NC4CC4)CC3)c3cn(C)nc23)cn2cc(C)nc(C)c12] |
| 420 | 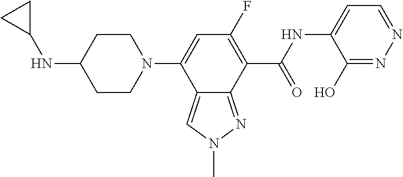 [SMILES: Cn1cc2c(N3CCC(NC4CC4)CC3)cc(F)c(C(=O)Nc3ccnnc3O)c2n1] |
| 421 | 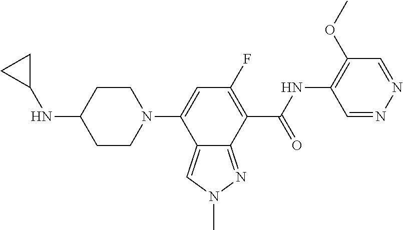 [SMILES: COc1cnncc1NC(=O)c1c(F)cc(N2CCC(NC3CC3)CC2)c2cn(C)nc12] |
| 422 | 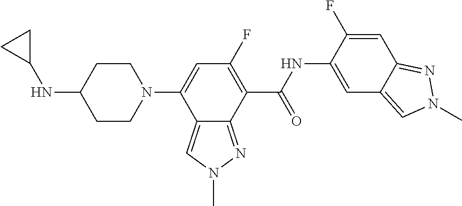 [SMILES: Cn1cc2cc(NC(=O)c3c(F)cc(N4CCC(NC5CC5)CC4)c4cn(C)nc34)c(F)cc2n1] |
| 423 | 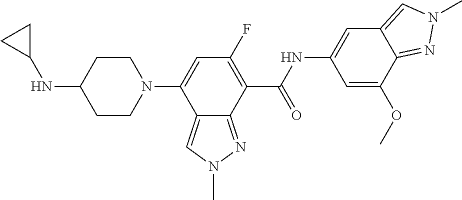 [SMILES: COc1cc(NC(=O)c2c(F)cc(N3CCC(NC4CC4)CC3)c3cn(C)nc23)cc2cn(C)nc12] |
| 424 | 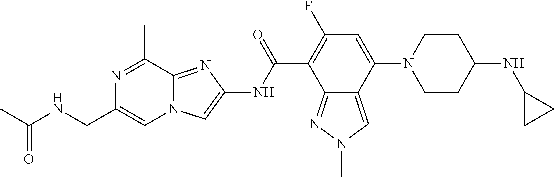 [SMILES: CC(=O)NCc1cn2cc(NC(=O)c3c(F)cc(N4CCC(NC5CC5)CC4)c4cn(C)nc34)nc2c(C)n1] |
| 425 | 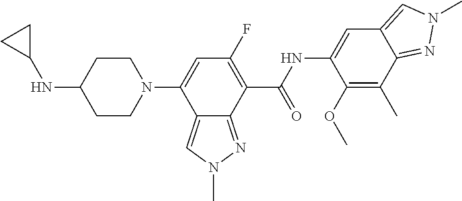 [SMILES: COc1c(NC(=O)c2c(F)cc(N3CCC(NC4CC4)CC3)c3cn(C)nc23)cc2cn(C)nc2c1C] |
| 426 | 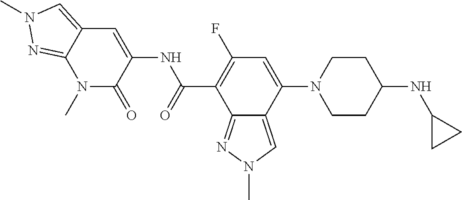 [SMILES: Cn1cc2c(N3CCC(NC4CC4)CC3)cc(F)c(C(=O)Nc3cc4cn(C)nc4n(C)c3=O)c2n1] |
| 427 | 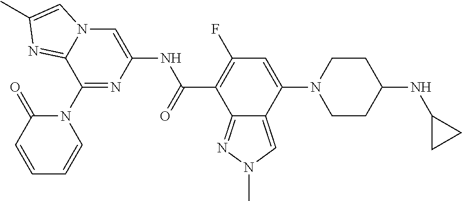 [SMILES: Cc1cn2cc(NC(=O)c3c(F)cc(N4CCC(NC5CC5)CC4)c4cn(C)nc34)nc(-n3ccccc3=O)c2n1] |
| 428 | 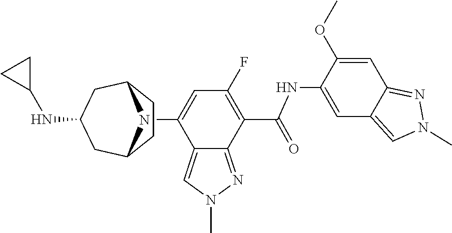 [SMILES: COc1cc2nn(C)cc2cc1NC(=O)c1c(F)cc(N2[C@@H]3CC[C@H]2C[C@@H](NC2CC2)C3)c2cn(C)nc12] |
| 429 | 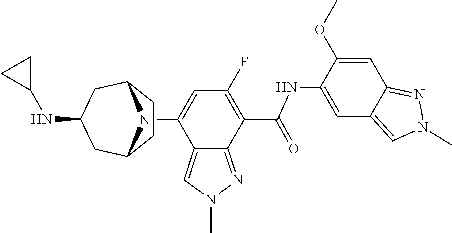 [SMILES: COc1cc2nn(C)cc2cc1NC(=O)c1c(F)cc(N2[C@@H]3CC[C@H]2C[C@H](NC2CC2)C3)c2cn(C)nc12] |
| 430 | 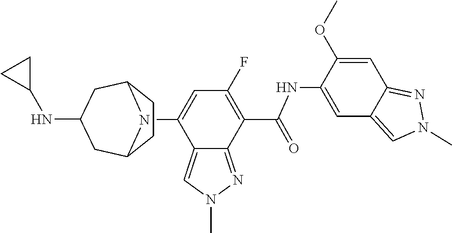 [SMILES: COc1cc2nn(C)cc2cc1NC(=O)c1c(F)cc(N2C3CCC2CC(NC2CC2)C3)c2cn(C)nc12] |
| 431 | 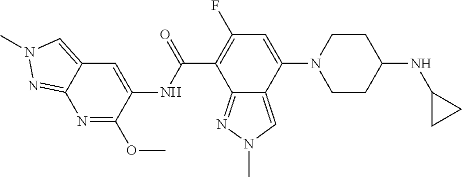 [SMILES: COc1nc2nn(C)cc2cc1NC(=O)c1c(F)cc(N2CCC(NC3CC3)CC2)c2cn(C)nc12] |
| 432 | 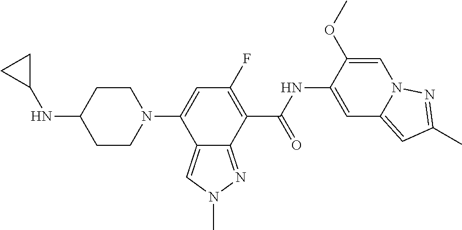 [SMILES: COc1cn2nc(C)cc2cc1NC(=O)c1c(F)cc(N2CCC(NC3CC3)CC2)c2cn(C)nc12] |
| 433 | 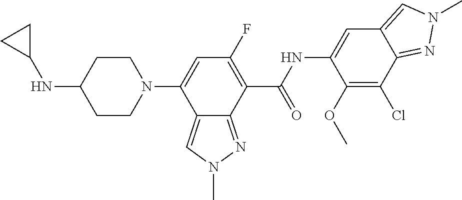 [SMILES: COc1c(NC(=O)c2c(F)cc(N3CCC(NC4CC4)CC3)c3cn(C)nc23)cc2cn(C)nc2c1Cl] |
| 434 | 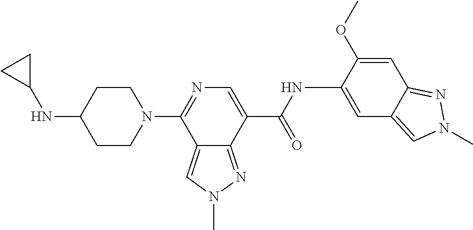 [SMILES: COc1cc2nn(C)cc2cc1NC(=O)c1cnc(N2CCC(NC3CC3)CC2)c2cn(C)nc12] |
| 435 | 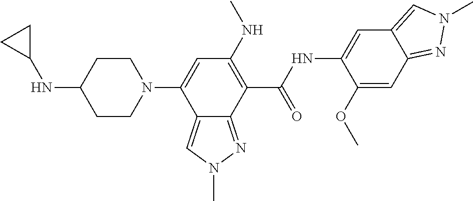 [SMILES: CNc1cc(N2CCC(NC3CC3)CC2)c2cn(C)nc2c1C(=O)Nc1cc2cn(C)nc2cc1OC] |
| 436 | 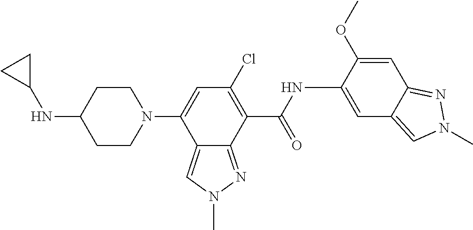 [SMILES: COc1cc2nn(C)cc2cc1NC(=O)c1c(Cl)cc(N2CCC(NC3CC3)CC2)c2cn(C)nc12] |
| 438 | 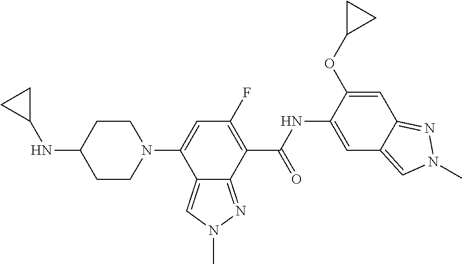 [SMILES: Cn1cc2cc(NC(=O)c3c(F)cc(N4CCC(NC5CC5)CC4)c4cn(C)nc34)c(OC3CC3)cc2n1] |
| 439 | 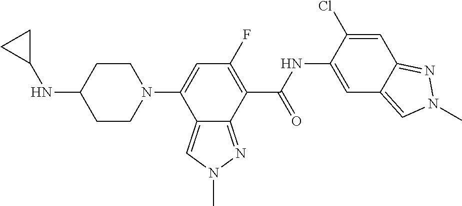 [SMILES: Cn1cc2cc(NC(=O)c3c(F)cc(N4CCC(NC5CC5)CC4)c4cn(C)nc34)c(Cl)cc2n1] |
| 440 | 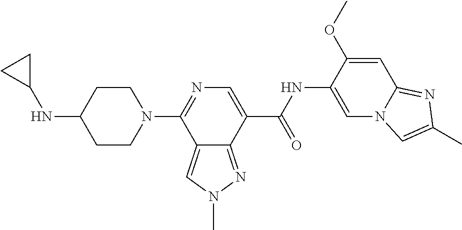 [SMILES: COc1cc2nc(C)cn2cc1NC(=O)c1cnc(N2CCC(NC3CC3)CC2)c2cn(C)nc12] |
| 441 | 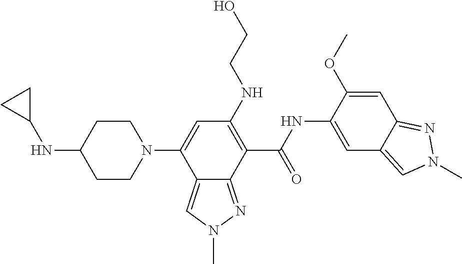 [SMILES: COc1cc2nn(C)cc2cc1NC(=O)c1c(NCCO)cc(N2CCC(NC3CC3)CC2)c2cn(C)nc12] |
| 442 | 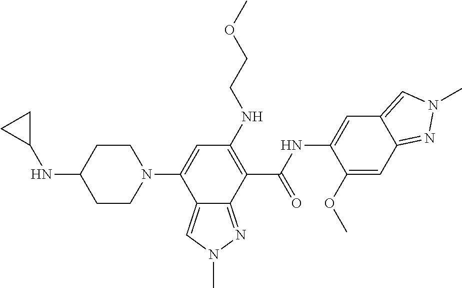 [SMILES: COCCNc1cc(N2CCC(NC3CC3)CC2)c2cn(C)nc2c1C(=O)Nc1cc2cn(C)nc2cc1OC] |
| 443 | 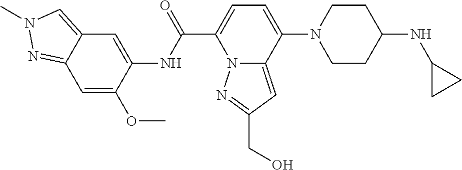 [SMILES: COc1cc2nn(C)cc2cc1NC(=O)c1ccc(N2CCC(NC3CC3)CC2)c2cc(CO)nn12] |
| 444 | 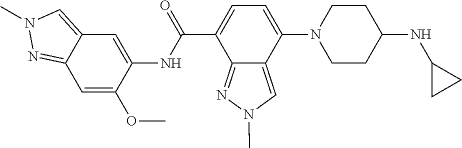 [SMILES: COc1cc2nn(C)cc2cc1NC(=O)c1ccc(N2CCC(NC3CC3)CC2)c2cn(C)nc12] |
| 445 | 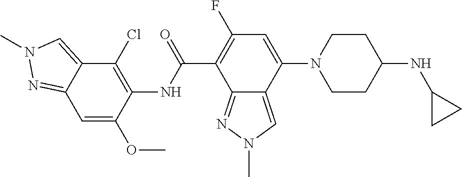 [SMILES: COc1cc2nn(C)cc2c(Cl)c1NC(=O)c1c(F)cc(N2CCC(NC3CC3)CC2)c2cn(C)nc12] |
| 447 | 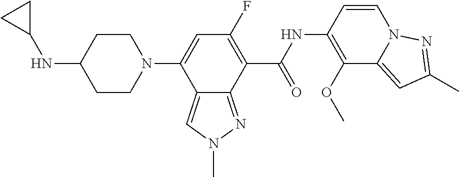 [SMILES: COc1c(NC(=O)c2c(F)cc(N3CCC(NC4CC4)CC3)c3cn(C)nc23)ccn2nc(C)cc12] |
| 448 | 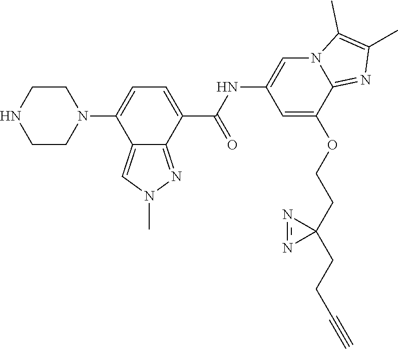 [SMILES: C#CCCC1(CCOc2cc(NC(=O)c3ccc(N4CCNCC4)c4cn(C)nc34)cn3c(C)c(C)nc23)N=N1] |
| 449 | 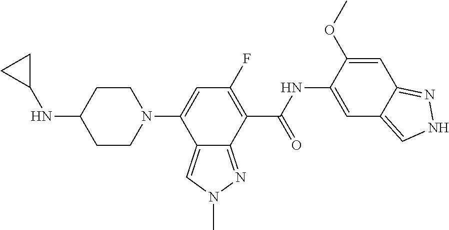 [SMILES: COc1cc2n[nH]cc2cc1NC(=O)c1c(F)cc(N2CCC(NC3CC3)CC2)c2cn(C)nc12] |
| 450 | 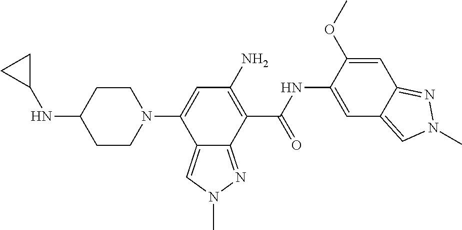 [SMILES: COc1cc2nn(C)cc2cc1NC(=O)c1c(N)cc(N2CCC(NC3CC3)CC2)c2cn(C)nc12] |
| 451 | 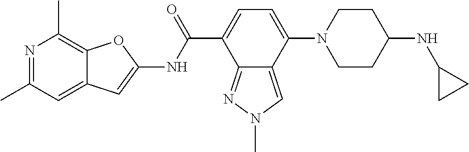 [SMILES: Cc1cc2cc(NC(=O)c3ccc(N4CCC(NC5CC5)CC4)c4cn(C)nc34)oc2c(C)n1] |
| 452 | 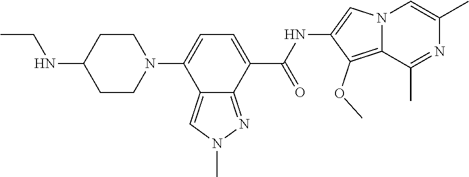 [SMILES: CCNC1CCN(c2ccc(C(=O)Nc3cn4cc(C)nc(C)c4c3OC)c3nn(C)cc23)CC1] |
| 453 | 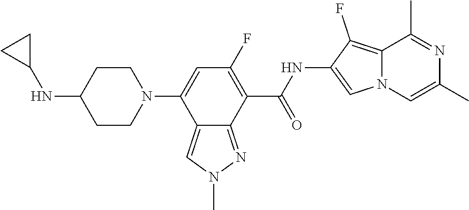 [SMILES: Cc1cn2cc(NC(=O)c3c(F)cc(N4CCC(NC5CC5)CC4)c4cn(C)nc34)c(F)c2c(C)n1] |
| 454 | 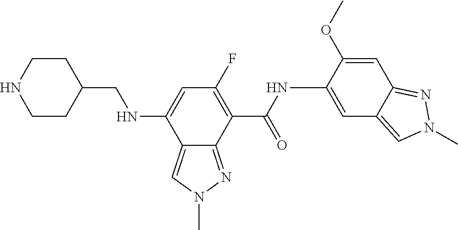 [SMILES: COc1cc2nn(C)cc2cc1NC(=O)c1c(F)cc(NCC2CCNCC2)c2cn(C)nc12] |
| 455 | 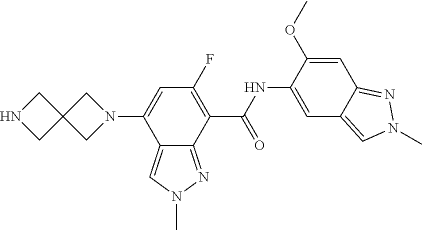 [SMILES: COc1cc2nn(C)cc2cc1NC(=O)c1c(F)cc(N2CC3(CNC3)C2)c2cn(C)nc12] |
| 456 | 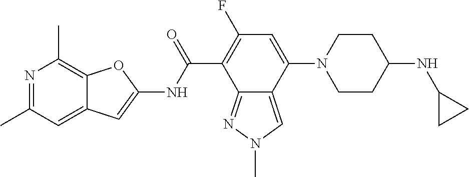 [SMILES: Cc1cc2cc(NC(=O)c3c(F)cc(N4CCC(NC5CC5)CC4)c4cn(C)nc34)oc2c(C)n1] |
| 457 | 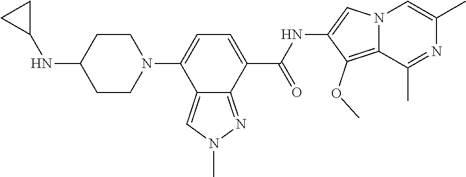 [SMILES: COc1c(NC(=O)c2ccc(N3CCC(NC4CC4)CC3)c3cn(C)nc23)cn2cc(C)nc(C)c12] |
| 458 | 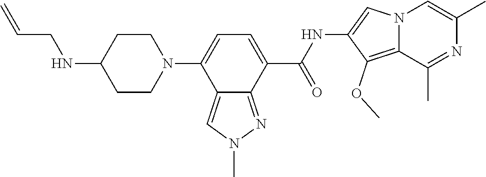 [SMILES: C=CCNC1CCN(c2ccc(C(=O)Nc3cn4cc(C)nc(C)c4c3OC)c3nn(C)cc23)CC1] |
| 459 | 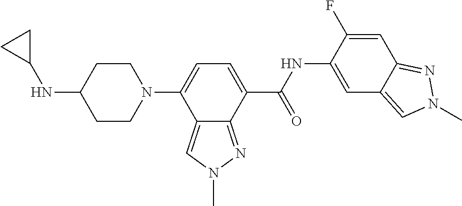 [SMILES: Cn1cc2cc(NC(=O)c3ccc(N4CCC(NC5CC5)CC4)c4cn(C)nc34)c(F)cc2n1] |
| 460 | 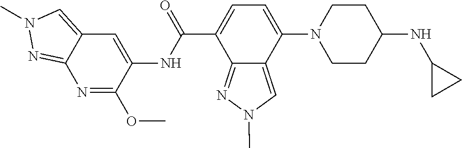 [SMILES: COc1nc2nn(C)cc2cc1NC(=O)c1ccc(N2CCC(NC3CC3)CC2)c2cn(C)nc12] |
| 461 | 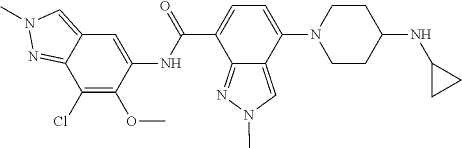 [SMILES: COc1c(NC(=O)c2ccc(N3CCC(NC4CC4)CC3)c3cn(C)nc23)cc2cn(C)nc2c1Cl] |
| 462 | 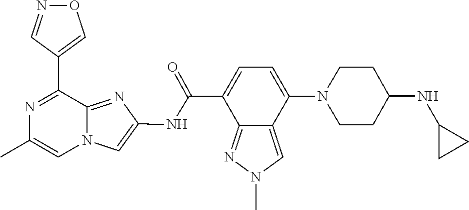 [SMILES: Cc1cn2cc(NC(=O)c3ccc(N4CCC(NC5CC5)CC4)c4cn(C)nc34)nc2c(-c2cnoc2)n1] |
| 463 | 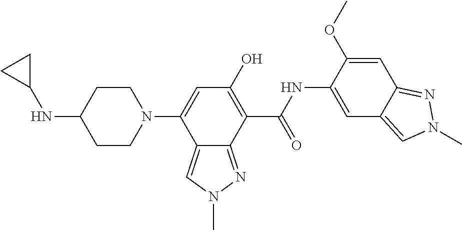 [SMILES: COc1cc2nn(C)cc2cc1NC(=O)c1c(O)cc(N2CCC(NC3CC3)CC2)c2cn(C)nc12] |
| 464 | 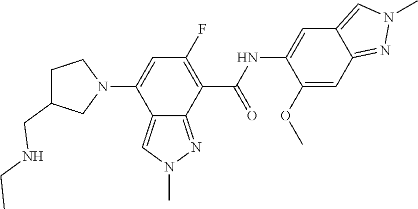 [SMILES: CCNCC1CCN(c2cc(F)c(C(=O)Nc3cc4cn(C)nc4cc3OC)c3nn(C)cc23)C1] |
| 465 | 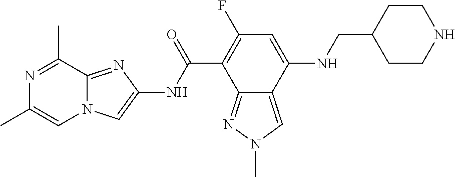 [SMILES: Cc1cn2cc(NC(=O)c3c(F)cc(NCC4CCNCC4)c4cn(C)nc34)nc2c(C)n1] |
| 466 | 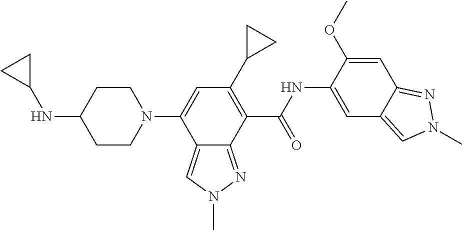 [SMILES: COc1cc2nn(C)cc2cc1NC(=O)c1c(C2CC2)cc(N2CCC(NC3CC3)CC2)c2cn(C)nc12] |
| 467 | 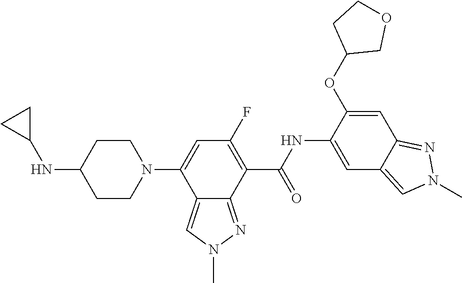 [SMILES: Cn1cc2cc(NC(=O)c3c(F)cc(N4CCC(NC5CC5)CC4)c4cn(C)nc34)c(OC3CCOC3)cc2n1] |
| 468 | 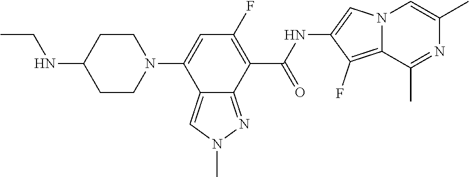 [SMILES: CCNC1CCN(c2cc(F)c(C(=O)Nc3cn4cc(C)nc(C)c4c3F)c3nn(C)cc23)CC1] |
| 469 | 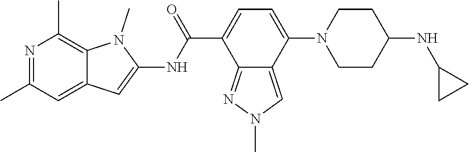 [SMILES: Cc1cc2cc(NC(=O)c3ccc(N4CCC(NC5CC5)CC4)c4cn(C)nc34)n(C)c2c(C)n1] |
| 470 | 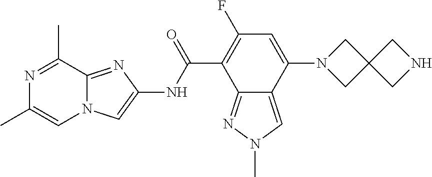 [SMILES: Cc1cn2cc(NC(=O)c3c(F)cc(N4CC5(CNC5)C4)c4cn(C)nc34)nc2c(C)n1] |
| 471 | 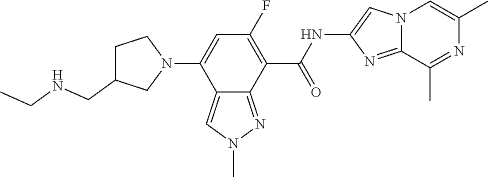 [SMILES: CCNCC1CCN(c2cc(F)c(C(=O)Nc3cn4cc(C)nc(C)c4n3)c3nn(C)cc23)C1] |
| 472 | 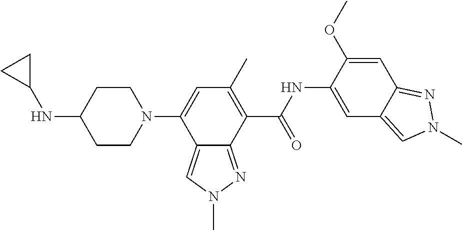 [SMILES: COc1cc2nn(C)cc2cc1NC(=O)c1c(C)cc(N2CCC(NC3CC3)CC2)c2cn(C)nc12] |
| 473 | 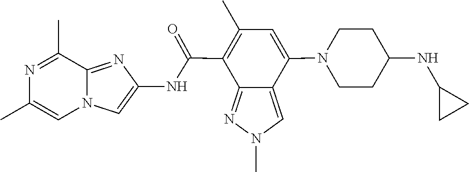 [SMILES: Cc1cn2cc(NC(=O)c3c(C)cc(N4CCC(NC5CC5)CC4)c4cn(C)nc34)nc2c(C)n1] |
| 474 | 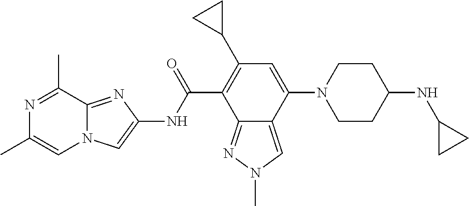 [SMILES: Cc1cn2cc(NC(=O)c3c(C4CC4)cc(N4CCC(NC5CC5)CC4)c4cn(C)nc34)nc2c(C)n1] |
| 475 | 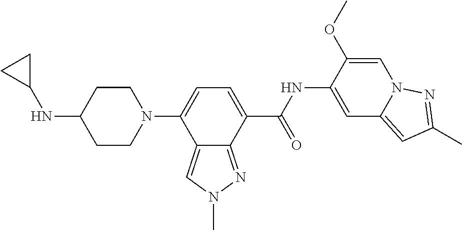 [SMILES: COc1cn2nc(C)cc2cc1NC(=O)c1ccc(N2CCC(NC3CC3)CC2)c2cn(C)nc12] |
| 476 | 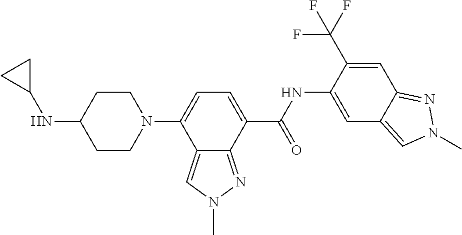 [SMILES: Cn1cc2cc(NC(=O)c3ccc(N4CCC(NC5CC5)CC4)c4cn(C)nc34)c(C(F)(F)F)cc2n1] |
| 477 | 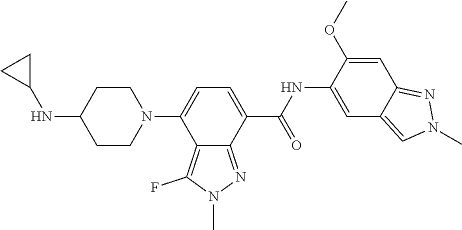 [SMILES: COc1cc2nn(C)cc2cc1NC(=O)c1ccc(N2CCC(NC3CC3)CC2)c2c(F)n(C)nc12] |
| 478 | 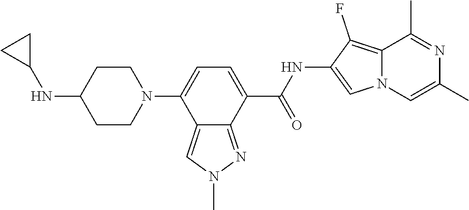 [SMILES: Cc1cn2cc(NC(=O)c3ccc(N4CCC(NC5CC5)CC4)c4cn(C)nc34)c(F)c2c(C)n1] |
| 479 | 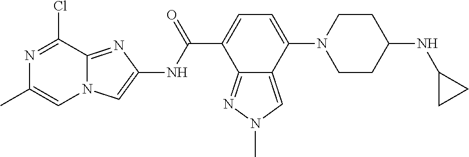 [SMILES: Cc1cn2cc(NC(=O)c3ccc(N4CCC(NC5CC5)CC4)c4cn(C)nc34)nc2c(Cl)n1] |
| 481 | 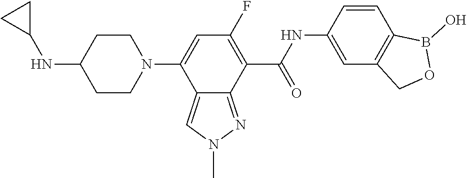 [SMILES: Cn1cc2c(N3CCC(NC4CC4)CC3)cc(F)c(C(=O)Nc3ccc4c(c3)COB4O)c2n1] |
| 482 | 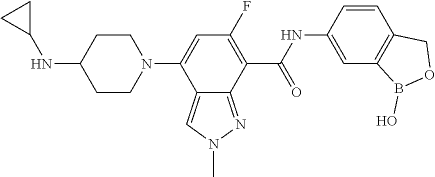 [SMILES: Cn1cc2c(N3CCC(NC4CC4)CC3)cc(F)c(C(=O)Nc3ccc4c(c3)B(O)OC4)c2n1] |
| 483 | 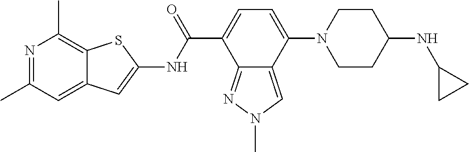 [SMILES: Cc1cc2cc(NC(=O)c3ccc(N4CCC(NC5CC5)CC4)c4cn(C)nc34)sc2c(C)n1] |
| 484 | 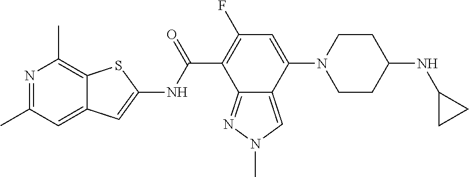 [SMILES: Cc1cc2cc(NC(=O)c3c(F)cc(N4CCC(NC5CC5)CC4)c4cn(C)nc34)sc2c(C)n1] |
| 485 | 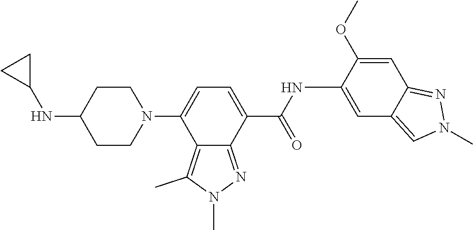 [SMILES: COc1cc2nn(C)cc2cc1NC(=O)c1ccc(N2CCC(NC3CC3)CC2)c2c(C)n(C)nc12] |
| 486 | 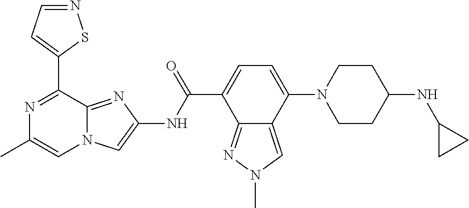 [SMILES: Cc1cn2cc(NC(=O)c3ccc(N4CCC(NC5CC5)CC4)c4cn(C)nc34)nc2c(-c2ccns2)n1] |
| 487 | 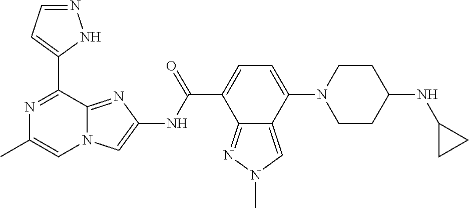 [SMILES: Cc1cn2cc(NC(=O)c3ccc(N4CCC(NC5CC5)CC4)c4cn(C)nc34)nc2c(-c2ccn[nH]2)n1] |
| 488 | 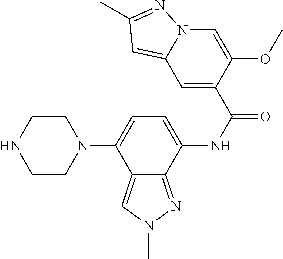 [SMILES: COc1cn2nc(C)cc2cc1C(=O)Nc1ccc(N2CCNCC2)c2cn(C)nc12] |
| 489 | 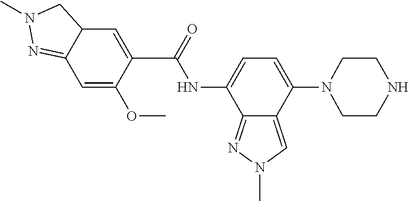 [SMILES: COC1=CC2=NN(C)CC2C=C1C(=O)Nc1ccc(N2CCNCC2)c2cn(C)nc12] |
| 490 | 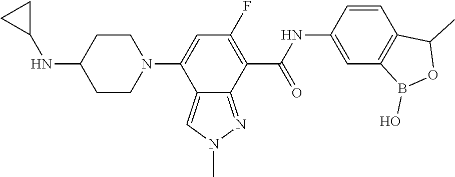 [SMILES: CC1OB(O)c2cc(NC(=O)c3c(F)cc(N4CCC(NC5CC5)CC4)c4cn(C)nc34)ccc21] |
| 491 | 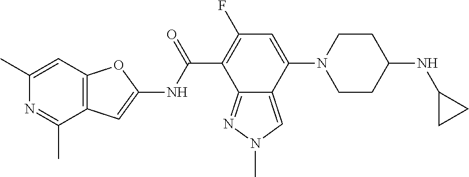 [SMILES: Cc1cc2oc(NC(=O)c3c(F)cc(N4CCC(NC5CC5)CC4)c4cn(C)nc34)cc2c(C)n1] |
| 492 | 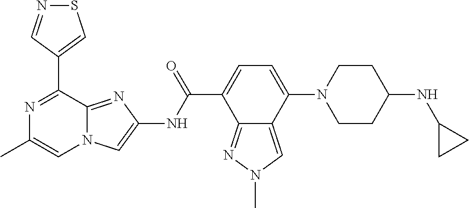 [SMILES: Cc1cn2cc(NC(=O)c3ccc(N4CCC(NC5CC5)CC4)c4cn(C)nc34)nc2c(-c2cnsc2)n1] |
| 493 | 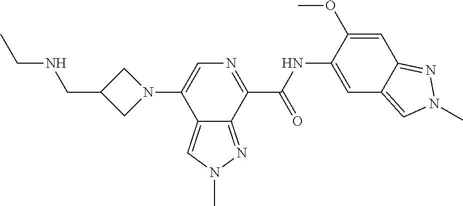 [SMILES: CCNCC1CN(c2cnc(C(=O)Nc3cc4cn(C)nc4cc3OC)c3nn(C)cc23)C1] |
| 494 | 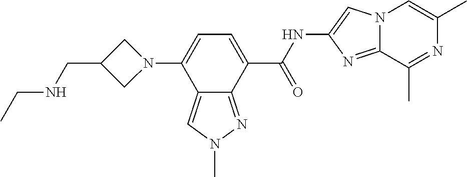 [SMILES: CCNCC1CN(c2ccc(C(=O)Nc3cn4cc(C)nc(C)c4n3)c3nn(C)cc23)C1] |
| 495 | 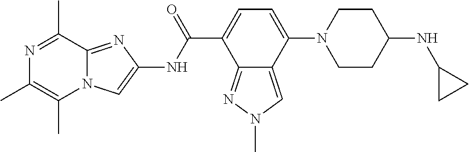 [SMILES: Cc1nc(C)c2nc(NC(=O)c3ccc(N4CCC(NC5CC5)CC4)c4cn(C)nc34)cn2c1C] |
| 496 | 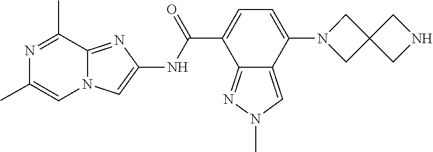 [SMILES: Cc1cn2cc(NC(=O)c3ccc(N4CC5(CNC5)C4)c4cn(C)nc34)nc2c(C)n1] |
| 497 | 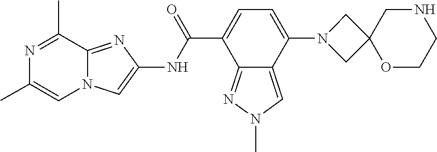 [SMILES: Cc1cn2cc(NC(=O)c3ccc(N4CC5(CNCCO5)C4)c4cn(C)nc34)nc2c(C)n1] |
| 498 | 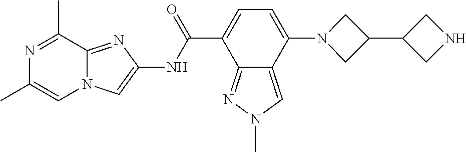 [SMILES: Cc1cn2cc(NC(=O)c3ccc(N4CC(C5CNC5)C4)c4cn(C)nc34)nc2c(C)n1] |
| 499 | 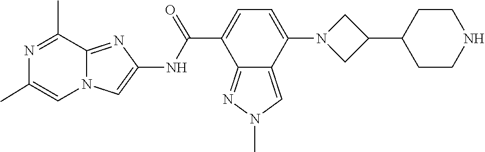 [SMILES: Cc1cn2cc(NC(=O)c3ccc(N4CC(C5CCNCC5)C4)c4cn(C)nc34)nc2c(C)n1] |
| 500 | 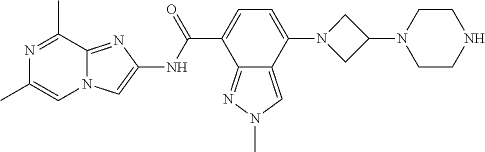 [SMILES: Cc1cn2cc(NC(=O)c3ccc(N4CC(N5CCNCC5)C4)c4cn(C)nc34)nc2c(C)n1] |
| 501 | 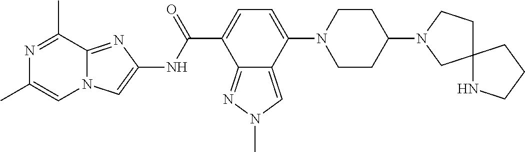 [SMILES: Cc1cn2cc(NC(=O)c3ccc(N4CCC(N5CCC6(CCCN6)C5)CC4)c4cn(C)nc34)nc2c(C)n1] |
| 502 | 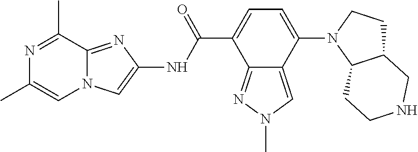 [SMILES: Cc1cn2cc(NC(=O)c3ccc(N4CC[C@@H]5CNCC[C@@H]54)c4cn(C)nc34)nc2c(C)n1] |
| 503 | 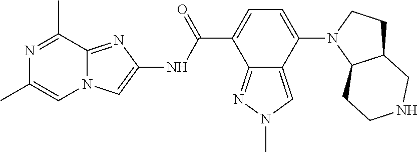 [SMILES: Cc1cn2cc(NC(=O)c3ccc(N4CC[C@H]5CNCC[C@H]54)c4cn(C)nc34)nc2c(C)n1] |
| 504 | 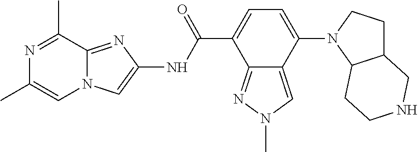 [SMILES: Cc1cn2cc(NC(=O)c3ccc(N4CCC5CNCCC54)c4cn(C)nc34)nc2c(C)n1] |
| 505 | 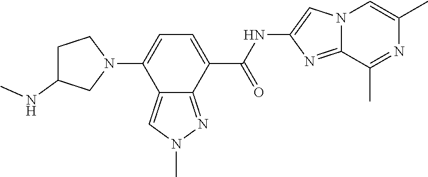 |
| 506 | 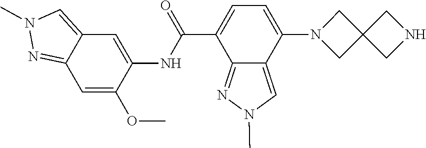 [SMILES: COc1cc2nn(C)cc2cc1NC(=O)c1ccc(N2CC3(CNC3)C2)c2cn(C)nc12] |
| 507 | 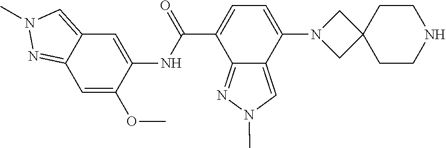 [SMILES: COc1cc2nn(C)cc2cc1NC(=O)c1ccc(N2CC3(CCNCC3)C2)c2cn(C)nc12] |
| 508 | 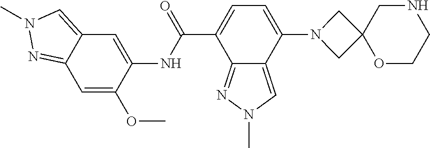 [SMILES: COc1cc2nn(C)cc2cc1NC(=O)c1ccc(N2CC3(CNCCO3)C2)c2cn(C)nc12] |
| 509 | 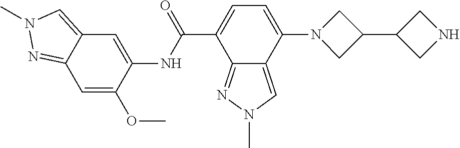 [SMILES: COc1cc2nn(C)cc2cc1NC(=O)c1ccc(N2CC(C3CNC3)C2)c2cn(C)nc12] |
| 510 | 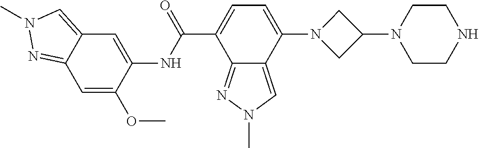 [SMILES: COc1cc2nn(C)cc2cc1NC(=O)c1ccc(N2CC(N3CCNCC3)C2)c2cn(C)nc12] |
| 511 | 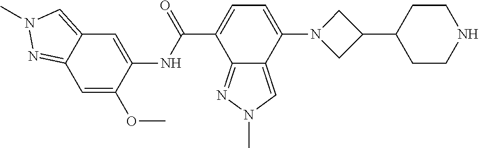 [SMILES: COc1cc2nn(C)cc2cc1NC(=O)c1ccc(N2CC(C3CCNCC3)C2)c2cn(C)nc12] |
| 512 | 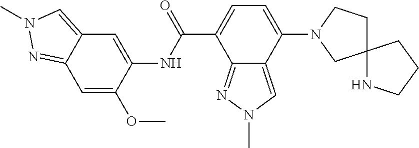 [SMILES: COc1cc2nn(C)cc2cc1NC(=O)c1ccc(N2CCC3(CCCN3)C2)c2cn(C)nc12] |
| 513 | 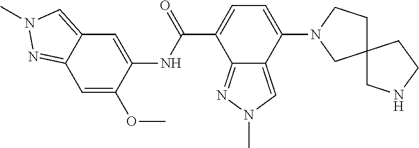 [SMILES: COc1cc2nn(C)cc2cc1NC(=O)c1ccc(N2CCC3(CCNC3)C2)c2cn(C)nc12] |
| 514 | 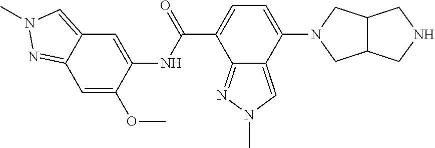 [SMILES: COc1cc2nn(C)cc2cc1NC(=O)c1ccc(N2CC3CNCC3C2)c2cn(C)nc12] |
| 515 | 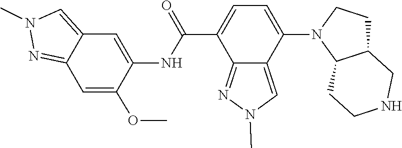 [SMILES: COc1cc2nn(C)cc2cc1NC(=O)c1ccc(N2CC[C@@H]3CNCC[C@@H]32)c2cn(C)nc12] |
| 516 | 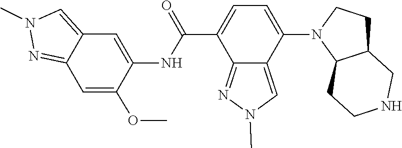 [SMILES: COc1cc2nn(C)cc2cc1NC(=O)c1ccc(N2CC[C@H]3CNCC[C@H]32)c2cn(C)nc12] |
| 517 | 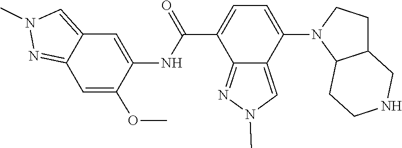 [SMILES: COc1cc2nn(C)cc2cc1NC(=O)c1ccc(N2CCC3CNCCC32)c2cn(C)nc12] |
| 518 | 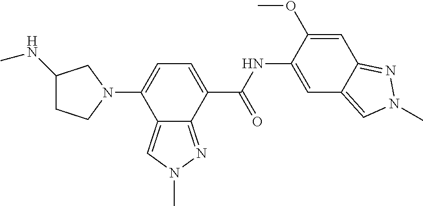 |
| 519 | 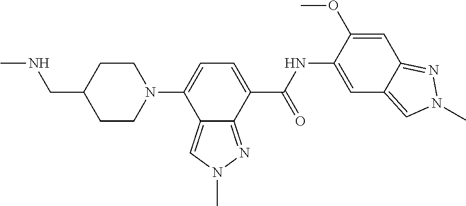 |
| 520 | 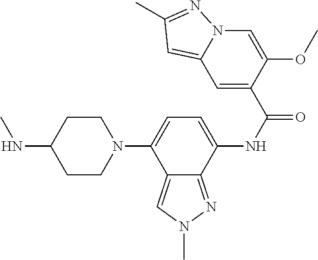 |
| 521 | 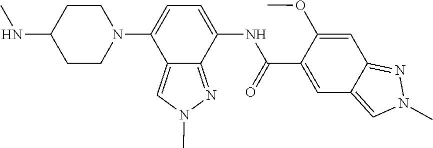 |
| 522 | 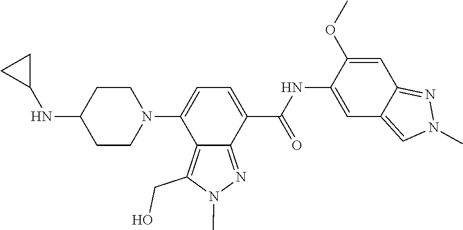 [SMILES: COc1cc2nn(C)cc2cc1NC(=O)c1ccc(N2CCC(NC3CC3)CC2)c2c(CO)n(C)nc12] |
| 523 | 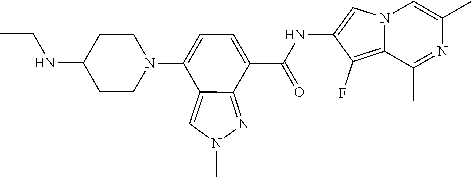 [SMILES: CCNC1CCN(c2ccc(C(=O)Nc3cn4cc(C)nc(C)c4c3F)c3nn(C)cc23)CC1] |
| 524 | 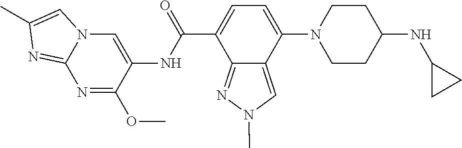 [SMILES: COc1nc2nc(C)cn2cc1NC(=O)c1ccc(N2CCC(NC3CC3)CC2)c2cn(C)nc12] |
| 525 | 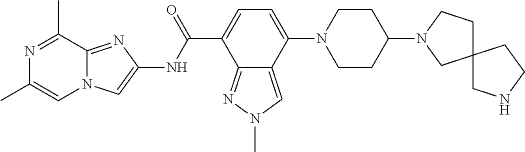 [SMILES: Cc1cn2cc(NC(=O)c3ccc(N4CCC(N5CCC6(CCNC6)C5)CC4)c4cn(C)nc34)nc2c(C)n1] |
| 526 | 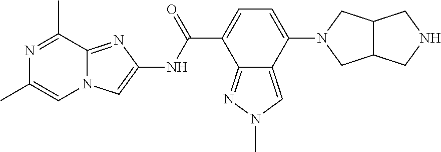 [SMILES: Cc1cn2cc(NC(=O)c3ccc(N4CC5CNCC5C4)c4cn(C)nc34)nc2c(C)n1] |
| 527 | 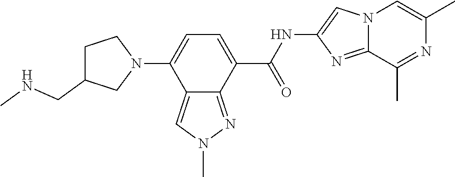 |
| 528 | 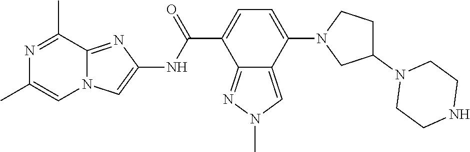 [SMILES: Cc1cn2cc(NC(=O)c3ccc(N4CCC(N5CCNCC5)C4)c4cn(C)nc34)nc2c(C)n1] |
| 529 | 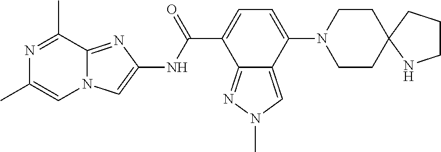 [SMILES: Cc1cn2cc(NC(=O)c3ccc(N4CCC5(CCCN5)CC4)c4cn(C)nc34)nc2c(C)n1] |
| 530 | 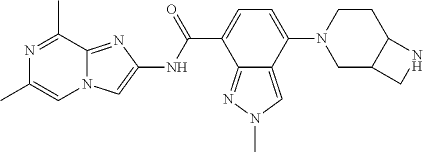 [SMILES: Cc1cn2cc(NC(=O)c3ccc(N4CCC5NCC5C4)c4cn(C)nc34)nc2c(C)n1] |
| 531 | 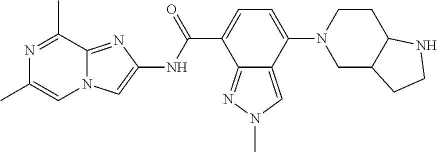 [SMILES: Cc1cn2cc(NC(=O)c3ccc(N4CCC5NCCC5C4)c4cn(C)nc34)nc2c(C)n1] |
| 532 | 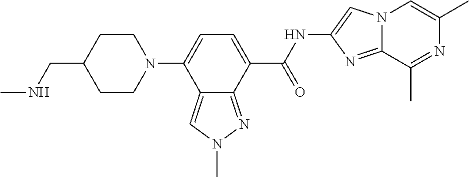 |
| 533 | 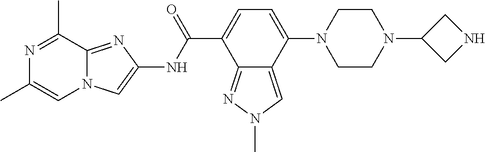 [SMILES: Cc1cn2cc(NC(=O)c3ccc(N4CCN(C5CNC5)CC4)c4cn(C)nc34)nc2c(C)n1] |
| 534 | 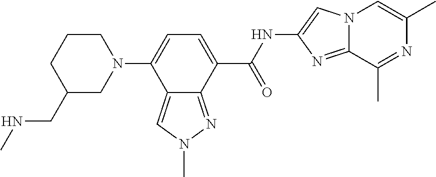 |
| 535 | 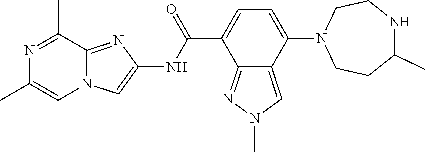 |
| 536 | 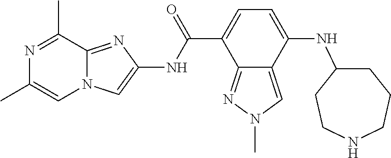 [SMILES: Cc1cn2cc(NC(=O)c3ccc(NC4CCCNCC4)c4cn(C)nc34)nc2c(C)n1] |
| 537 | 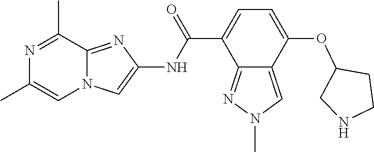 [SMILES: Cc1cn2cc(NC(=O)c3ccc(OC4CCNC4)c4cn(C)nc34)nc2c(C)n1] |
| 538 | 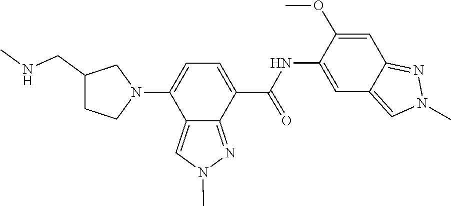 |
| 539 | 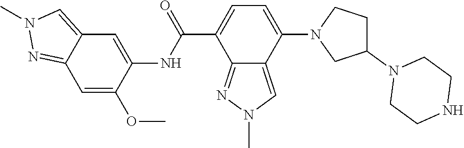 [SMILES: COc1cc2nn(C)cc2cc1NC(=O)c1ccc(N2CCC(N3CCNCC3)C2)c2cn(C)nc12] |
| 540 | 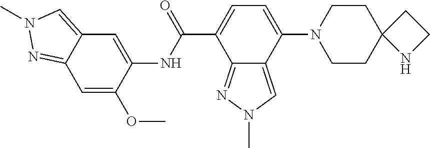 [SMILES: COc1cc2nn(C)cc2cc1NC(=O)c1ccc(N2CCC3(CCN3)CC2)c2cn(C)nc12] |
| 541 | 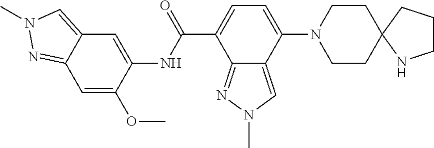 [SMILES: COc1cc2nn(C)cc2cc1NC(=O)c1ccc(N2CCC3(CCCN3)CC2)c2cn(C)nc12] |
| 542 | 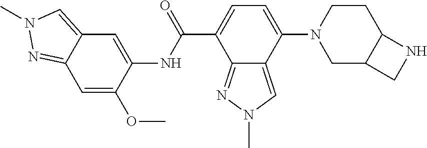 [SMILES: COc1cc2nn(C)cc2cc1NC(=O)c1ccc(N2CCC3NCC3C2)c2cn(C)nc12] |
| 543 | 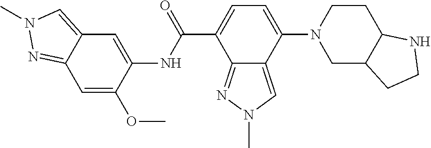 [SMILES: COc1cc2nn(C)cc2cc1NC(=O)c1ccc(N2CCC3NCCC3C2)c2cn(C)nc12] |
| 544 | 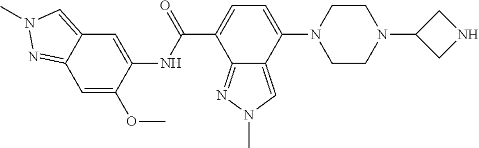 [SMILES: COc1cc2nn(C)cc2cc1NC(=O)c1ccc(N2CCN(C3CNC3)CC2)c2cn(C)nc12] |
| 545 | 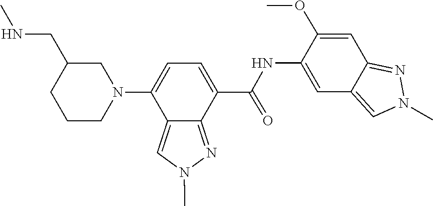 |
| 546 | 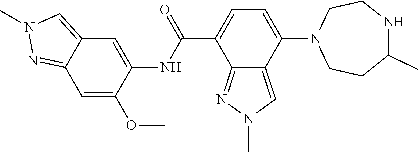 |
| 547 | 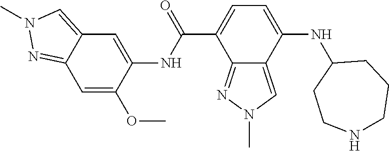 [SMILES: COc1cc2nn(C)cc2cc1NC(=O)c1ccc(NC2CCCNCC2)c2cn(C)nc12] |
| 548 | 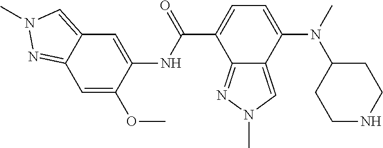 [SMILES: COc1cc2nn(C)cc2cc1NC(=O)c1ccc(N(C)C2CCNCC2)c2cn(C)nc12] |
| 549 | 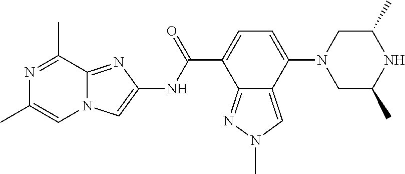 |
| 550 | 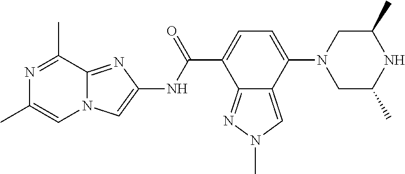 |
| 551 | 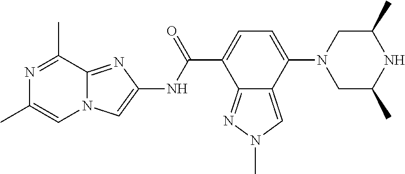 |
| 552 | 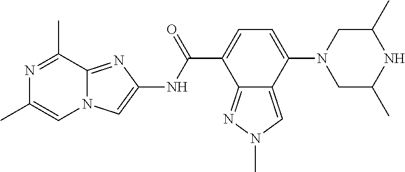 |
| 553 | 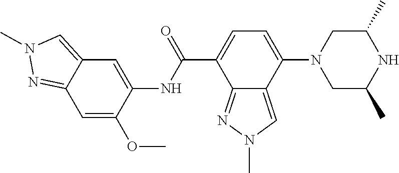 |
| 554 | 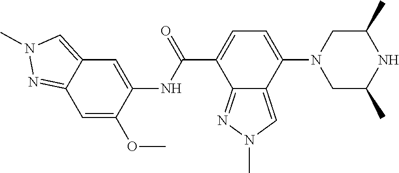 |
| 555 | 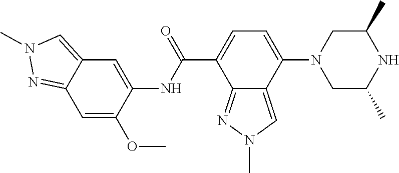 |
| 556 | 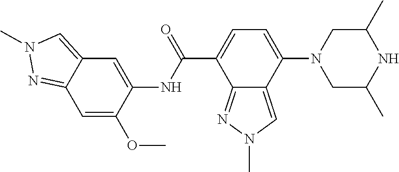 |
| 557 | 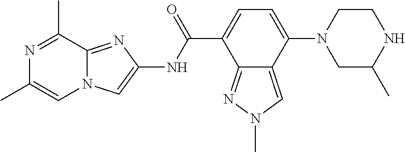 |
| 558 | 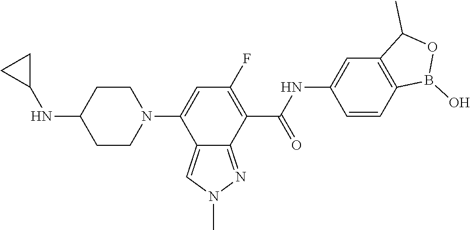 [SMILES: CC1OB(O)c2ccc(NC(=O)c3c(F)cc(N4CCC(NC5CC5)CC4)c4cn(C)nc34)cc21] |
| 559 | 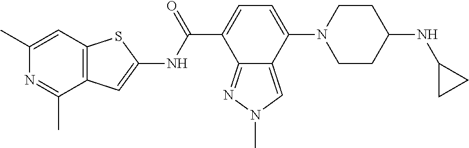 [SMILES: Cc1cc2sc(NC(=O)c3ccc(N4CCC(NC5CC5)CC4)c4cn(C)nc34)cc2c(C)n1] |
| 560 | 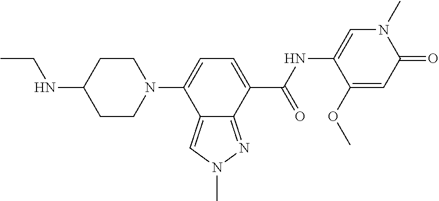 [SMILES: CCNC1CCN(c2ccc(C(=O)Nc3cn(C)c(=O)cc3OC)c3nn(C)cc23)CC1] |
| 561 | 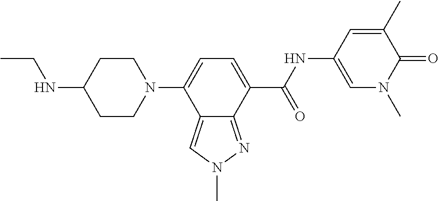 [SMILES: CCNC1CCN(c2ccc(C(=O)Nc3cc(C)c(=O)n(C)c3)c3nn(C)cc23)CC1] |
| 562 | 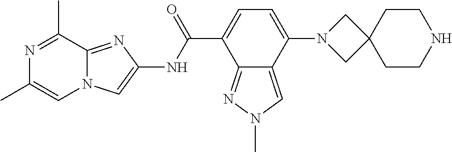 [SMILES: Cc1cn2cc(NC(=O)c3ccc(N4CC5(CCNCC5)C4)c4cn(C)nc34)nc2c(C)n1] |
| 563 | 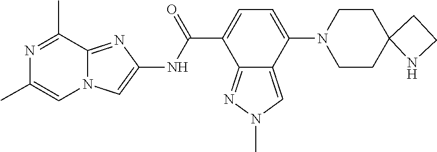 [SMILES: Cc1cn2cc(NC(=O)c3ccc(N4CCC5(CCN5)CC4)c4cn(C)nc34)nc2c(C)n1] |
| 564 | 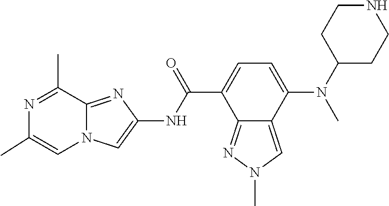 [SMILES: Cc1cn2cc(NC(=O)c3ccc(N(C)C4CCNCC4)c4cn(C)nc34)nc2c(C)n1] |
| 565 | 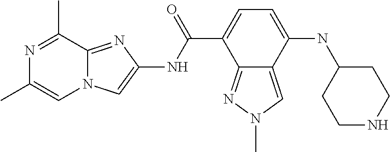 [SMILES: Cc1cn2cc(NC(=O)c3ccc(NC4CCNCC4)c4cn(C)nc34)nc2c(C)n1] |
| 566 | 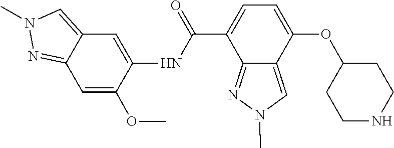 [SMILES: COc1cc2nn(C)cc2cc1NC(=O)c1ccc(OC2CCNCC2)c2cn(C)nc12] |
| 567 | 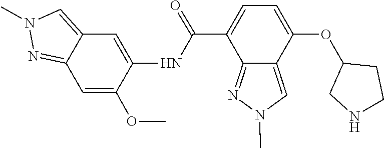 [SMILES: COc1cc2nn(C)cc2cc1NC(=O)c1ccc(OC2CCNC2)c2cn(C)nc12] |
| 568 | 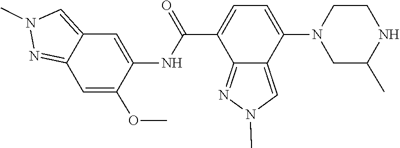 |
| 569 | 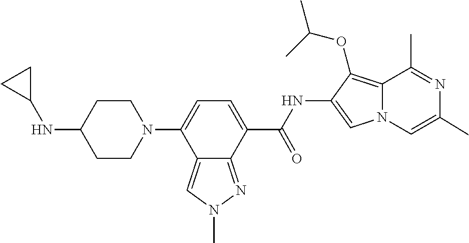 [SMILES: Cc1cn2cc(NC(=O)c3ccc(N4CCC(NC5CC5)CC4)c4cn(C)nc34)c(OC(C)C)c2c(C)n1] |
| 570 | 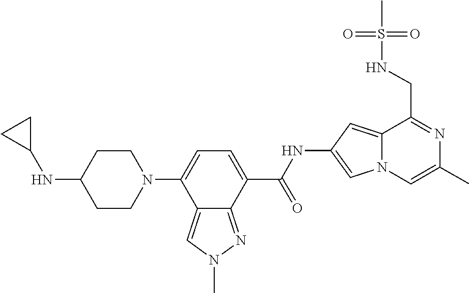 [SMILES: Cc1cn2cc(NC(=O)c3ccc(N4CCC(NC5CC5)CC4)c4cn(C)nc34)cc2c(CNS(C)(=O)=O)n1] |
| 571 | 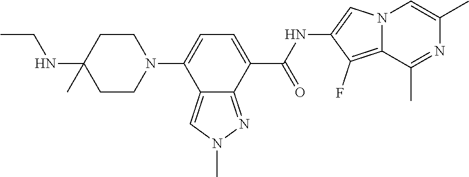 [SMILES: CCNC1(C)CCN(c2ccc(C(=O)Nc3cn4cc(C)nc(C)c4c3F)c3nn(C)cc23)CC1] |
| 572 | 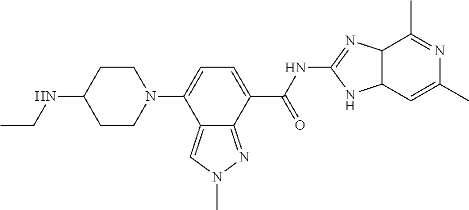 [SMILES: CCNC1CCN(c2ccc(C(=O)NC3=NC4C(C)=NC(C)=CC4N3)c3nn(C)cc23)CC1] |
| 573 | 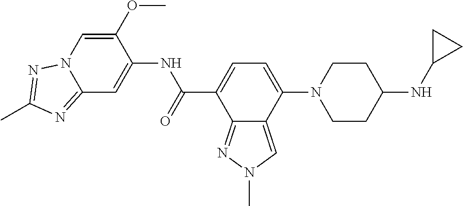 [SMILES: COc1cn2nc(C)nc2cc1NC(=O)c1ccc(N2CCC(NC3CC3)CC2)c2cn(C)nc12] |
| 574 | 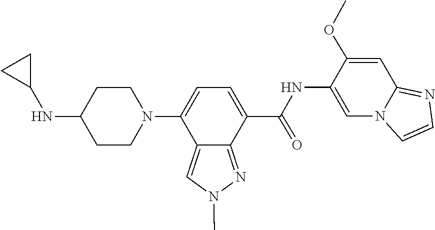 [SMILES: COc1cc2nccn2cc1NC(=O)c1ccc(N2CCC(NC3CC3)CC2)c2cn(C)nc12] |
| 575 | 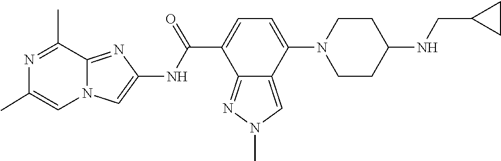 [SMILES: Cc1cn2cc(NC(=O)c3ccc(N4CCC(NCC5CC5)CC4)c4cn(C)nc34)nc2c(C)n1] |
| 576 | 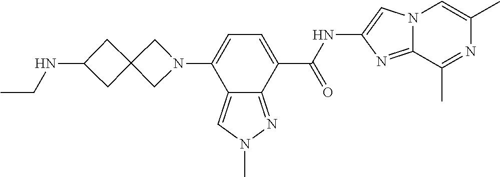 [SMILES: CCNC1CC2(C1)CN(c1ccc(C(=O)Nc3cn4cc(C)nc(C)c4n3)c3nn(C)cc13)C2] |
| 577 | 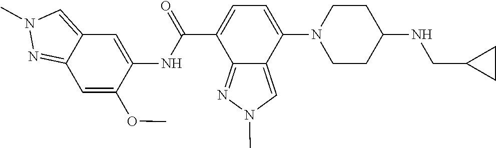 [SMILES: COc1cc2nn(C)cc2cc1NC(=O)c1ccc(N2CCC(NCC3CC3)CC2)c2cn(C)nc12] |
| 578 | 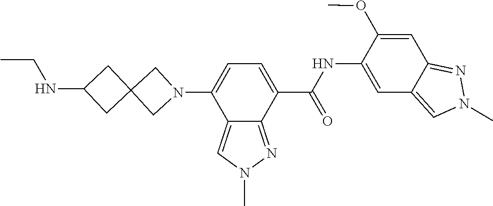 [SMILES: CCNC1CC2(C1)CN(c1ccc(C(=O)Nc3cc4cn(C)nc4cc3OC)c3nn(C)cc13)C2] |
| 579 | 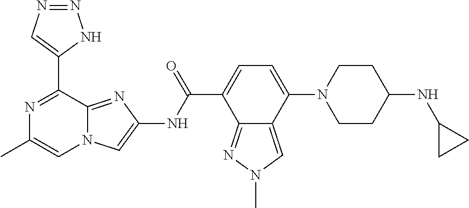 [SMILES: Cc1cn2cc(NC(=O)c3ccc(N4CCC(NC5CC5)CC4)c4cn(C)nc34)nc2c(-c2cnn[nH]2)n1] |
| 580 | 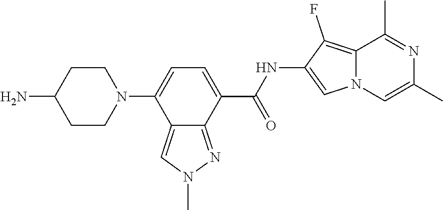 [SMILES: Cc1cn2cc(NC(=O)c3ccc(N4CCC(N)CC4)c4cn(C)nc34)c(F)c2c(C)n1] |
| 581 | 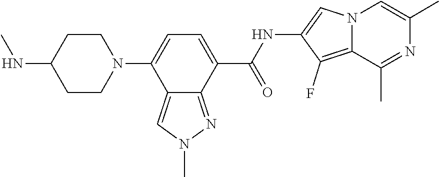 |
| 582 | 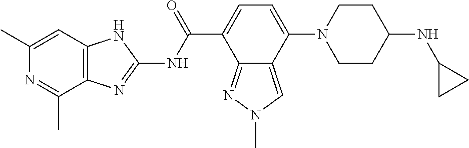 [SMILES: Cc1cc2[nH]c(NC(=O)c3ccc(N4CCC(NC5CC5)CC4)c4cn(C)nc34)nc2c(C)n1] |
| 583 | 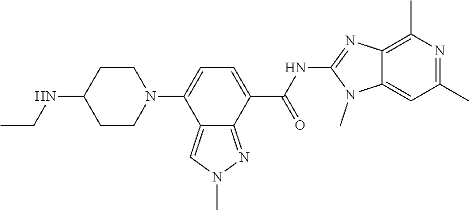 [SMILES: CCNC1CCN(c2ccc(C(=O)Nc3nc4c(C)nc(C)cc4n3C)c3nn(C)cc23)CC1] |
| 584 | 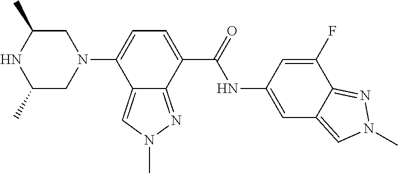 |
| 585 | 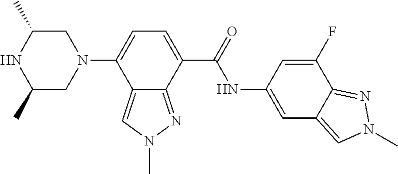 [SMILES: C[C@@H]1CN(c2ccc(C(=O)Nc3cc(F)c4nn(C)cc4c3)c3nn(C)cc23)C[C@@H](C)N1] |
| 586 | 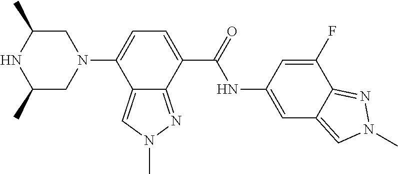 |
| 587 | 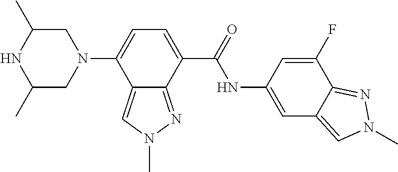 |
| 588 | 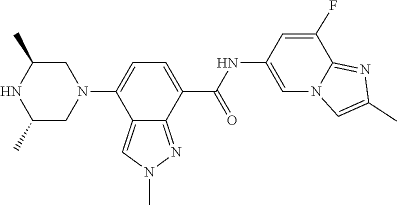 [SMILES: Cc1cn2cc(NC(=O)c3ccc(N4C[C@H](C)N[C@@H](C)C4)c4cn(C)nc34)cc(F)c2n1] |
| 589 | 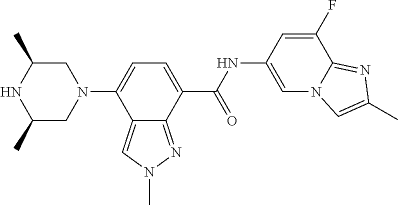 [SMILES: Cc1cn2cc(NC(=O)c3ccc(N4C[C@@H](C)N[C@@H](C)C4)c4cn(C)nc34)cc(F)c2n1] |
| 590 | 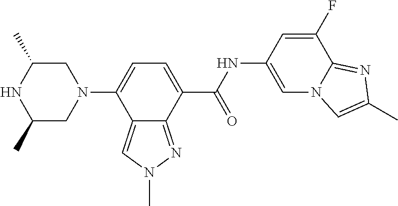 [SMILES: Cc1cn2cc(NC(=O)c3ccc(N4C[C@@H](C)N[C@H](C)C4)c4cn(C)nc34)cc(F)c2n1] |
| 591 | 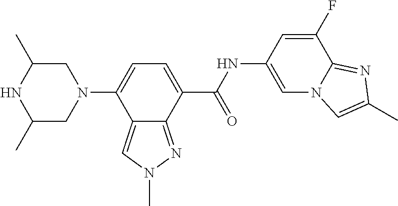 [SMILES: Cc1cn2cc(NC(=O)c3ccc(N4CC(C)NC(C)C4)c4cn(C)nc34)cc(F)c2n1] |
| 592 | 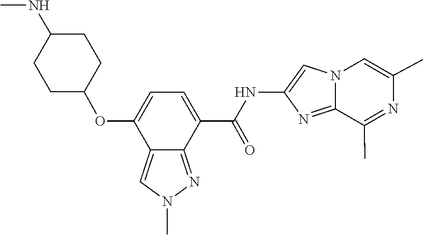 |
| 593 | 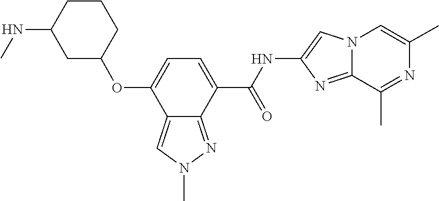 |
| 594 | 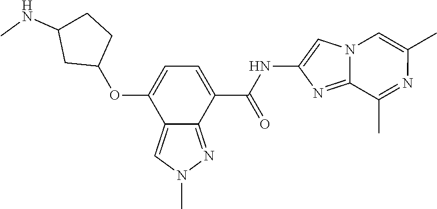 |
| 595 | 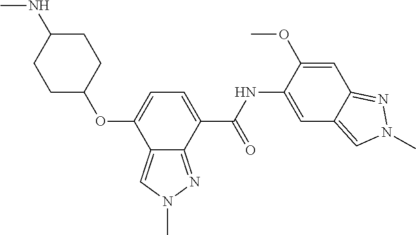 |
| 596 | 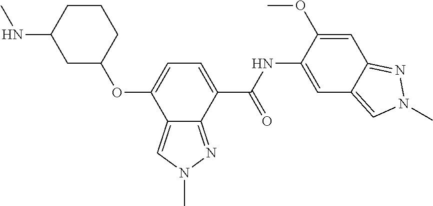 |
| 597 | 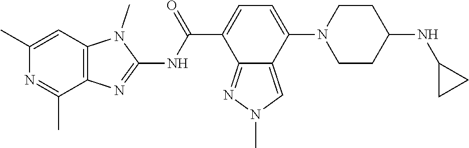 [SMILES: Cc1cc2c(nc(NC(=O)c3ccc(N4CCC(NC5CC5)CC4)c4cn(C)nc34)n2C)c(C)n1] |
| 598 | 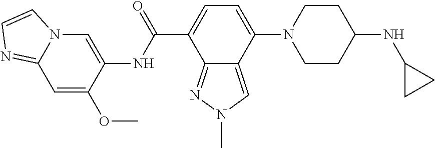 [SMILES: COc1cc2nccn2cc1NC(=O)c1ccc(N2CCC(NC3CC3)CC2)c2cn(C)nc12] |
| 599 | 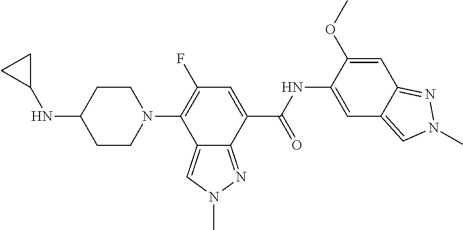 [SMILES: COc1cc2nn(C)cc2cc1NC(=O)c1cc(F)c(N2CCC(NC3CC3)CC2)c2cn(C)nc12] |
| 600 | 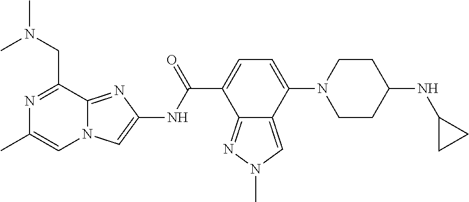 [SMILES: Cc1cn2cc(NC(=O)c3ccc(N4CCC(NC5CC5)CC4)c4cn(C)nc34)nc2c(CN(C)C)n1] |
| 601 | 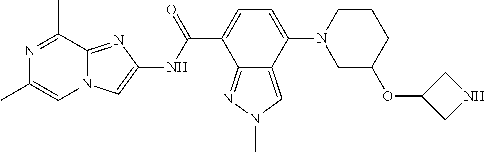 [SMILES: Cc1cn2cc(NC(=O)c3ccc(N4CCCC(OC5CNC5)C4)c4cn(C)nc34)nc2c(C)n1] |
| 602 | 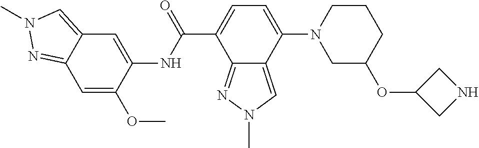 [SMILES: COc1cc2nn(C)cc2cc1NC(=O)c1ccc(N2CCCC(OC3CNC3)C2)c2cn(C)nc12] |
| 603 | 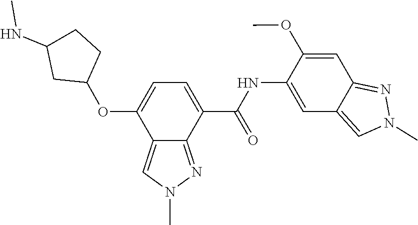 |
| 604 | 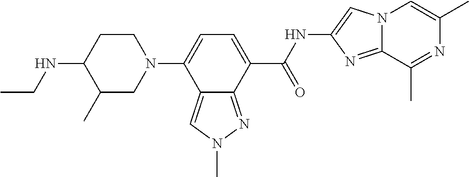 [SMILES: CCNC1CCN(c2ccc(C(=O)Nc3cn4cc(C)nc(C)c4n3)c3nn(C)cc23)CC1C] |
| 605 | 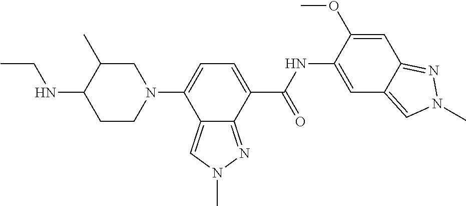 [SMILES: CCNC1CCN(c2ccc(C(=O)Nc3cc4cn(C)nc4cc3OC)c3nn(C)cc23)CC1C] |
| 606 | 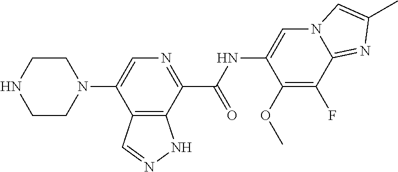 [SMILES: COc1c(NC(=O)c2ncc(N3CCNCC3)c3cn[nH]c23)cn2cc(C)nc2c1F] |
| 607 | 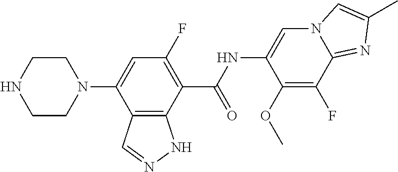 [SMILES: COc1c(NC(=O)c2c(F)cc(N3CCNCC3)c3cn[nH]c23)cn2cc(C)nc2c1F] |
| 608 | 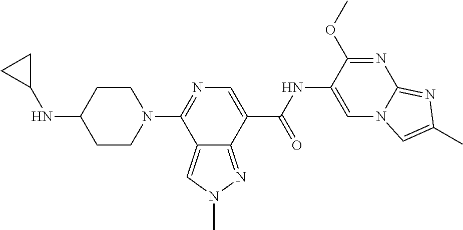 [SMILES: COc1nc2nc(C)cn2cc1NC(=O)c1cnc(N2CCC(NC3CC3)CC2)c2cn(C)nc12] |
| 609 | 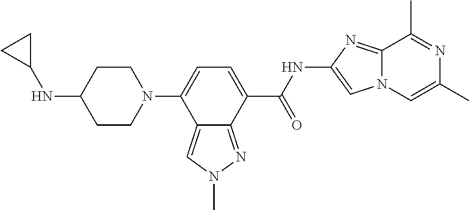 [SMILES: Cc1cn2cc(NC(=O)c3ccc(N4CCC(NC5CC5)CC4)c4cn(C)nc34)nc2c(C)n1] |
| 610 | 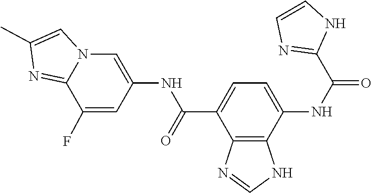 [SMILES: Cc1cn2cc(NC(=O)c3ccc(NC(=O)c4ncc[nH]4)c4[nH]cnc34)cc(F)c2n1] |
| 611 | 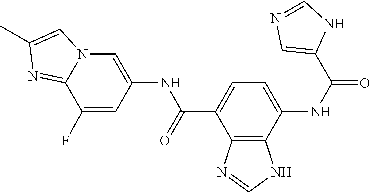 [SMILES: Cc1cn2cc(NC(=O)c3ccc(NC(=O)c4cnc[nH]4)c4[nH]cnc34)cc(F)c2n1] |
| 612 | 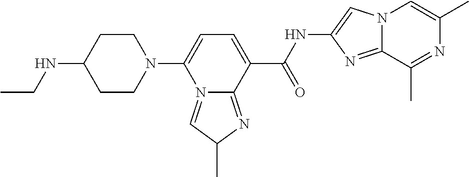 |
| 613 | 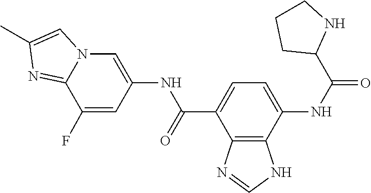 [SMILES: Cc1cn2cc(NC(=O)c3ccc(NC(=O)C4CCCN4)c4[nH]cnc34)cc(F)c2n1] |
| 614 | 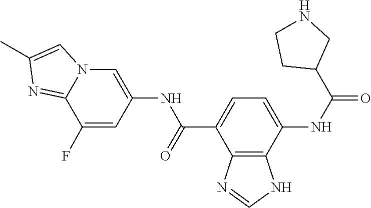 [SMILES: Cc1cn2cc(NC(=O)c3ccc(NC(=O)C4CCNC4)c4[nH]cnc34)cc(F)c2n1] |
| 615 | 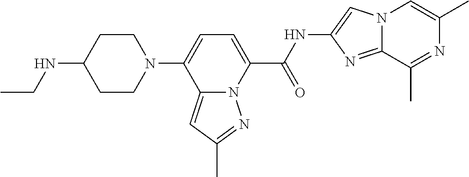 [SMILES: CCNC1CCN(c2ccc(C(=O)Nc3cn4cc(C)nc(C)c4n3)n3nc(C)cc23)CC1] |
| 616 | 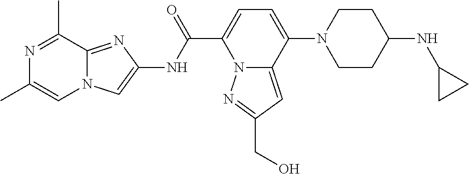 [SMILES: Cc1cn2cc(NC(=O)c3ccc(N4CCC(NC5CC5)CC4)c4cc(CO)nn34)nc2c(C)n1] |
| 617 | 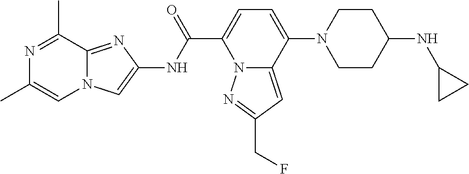 [SMILES: Cc1cn2cc(NC(=O)c3ccc(N4CCC(NC5CC5)CC4)c4cc(CF)nn34)nc2c(C)n1] |
| 618 | 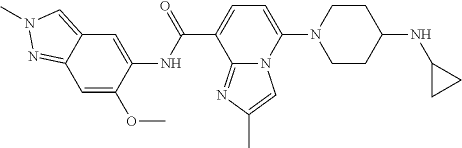 [SMILES: COc1cc2nn(C)cc2cc1NC(=O)c1ccc(N2CCC(NC3CC3)CC2)n2cc(C)nc12] |
| 619 | 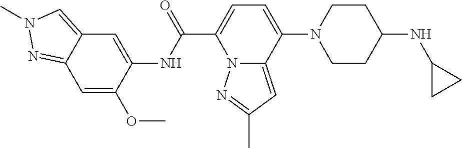 [SMILES: COc1cc2nn(C)cc2cc1NC(=O)c1ccc(N2CCC(NC3CC3)CC2)c2cc(C)nn12] |
| 620 | 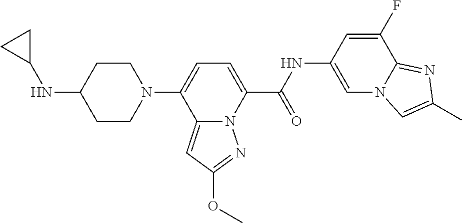 [SMILES: COc1cc2c(N3CCC(NC4CC4)CC3)ccc(C(=O)Nc3cc(F)c4nc(C)cn4c3)n2n1] |
| 621 | 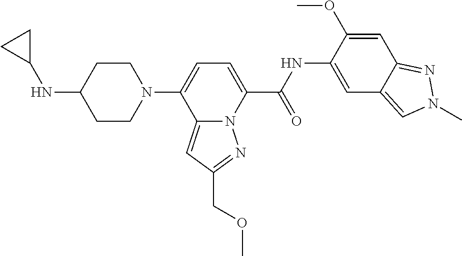 [SMILES: COCc1cc2c(N3CCC(NC4CC4)CC3)ccc(C(=O)Nc3cc4cn(C)nc4cc3OC)n2n1] |
| 622 | 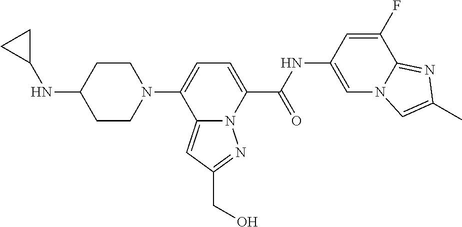 [SMILES: Cc1cn2cc(NC(=O)c3ccc(N4CCC(NC5CC5)CC4)c4cc(CO)nn34)cc(F)c2n1] |
| 623 | 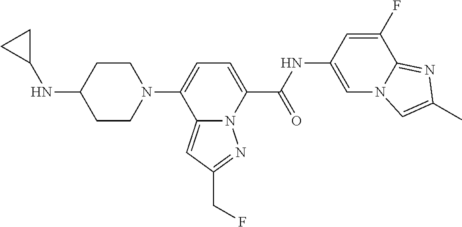 [SMILES: Cc1cn2cc(NC(=O)c3ccc(N4CCC(NC5CC5)CC4)c4cc(CF)nn34)cc(F)c2n1] |
| 624 | 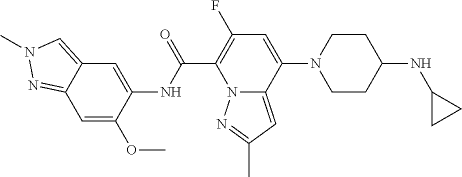 [SMILES: COc1cc2nn(C)cc2cc1NC(=O)c1c(F)cc(N2CCC(NC3CC3)CC2)c2cc(C)nn12] |
| 625 | 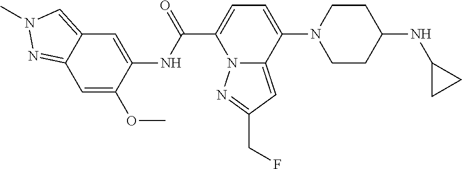 [SMILES: COc1cc2nn(C)cc2cc1NC(=O)c1ccc(N2CCC(NC3CC3)CC2)c2cc(CF)nn12] |
| 626 | 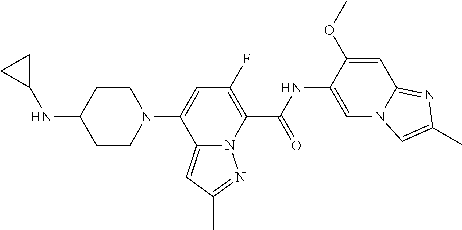 [SMILES: COc1cc2nc(C)cn2cc1NC(=O)c1c(F)cc(N2CCC(NC3CC3)CC2)c2cc(C)nn12] |
| 627 | 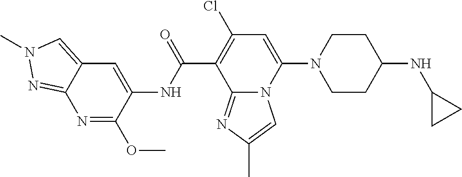 [SMILES: COc1nc2nn(C)cc2cc1NC(=O)c1c(Cl)cc(N2CCC(NC3CC3)CC2)n2cc(C)nc12] |
| 628 | 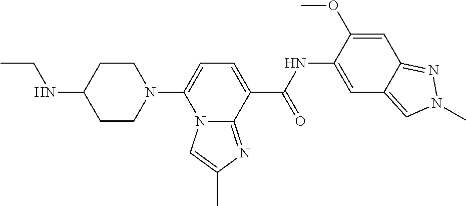 [SMILES: CCNC1CCN(c2ccc(C(=O)Nc3cc4cn(C)nc4cc3OC)c3nc(C)cn23)CC1] |
| 629 | 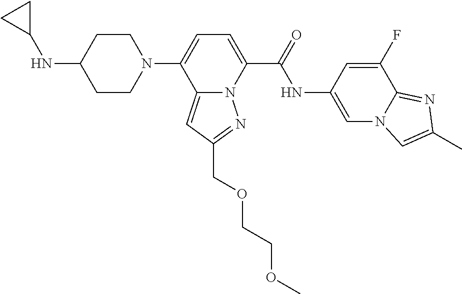 [SMILES: COCCOCc1cc2c(N3CCC(NC4CC4)CC3)ccc(C(=O)Nc3cc(F)c4nc(C)cn4c3)n2n1] |
| 630 | 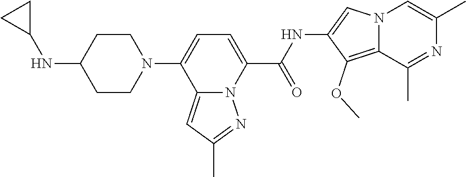 [SMILES: COc1c(NC(=O)c2ccc(N3CCC(NC4CC4)CC3)c3cc(C)nn23)cn2cc(C)nc(C)c12] |
| 631 | 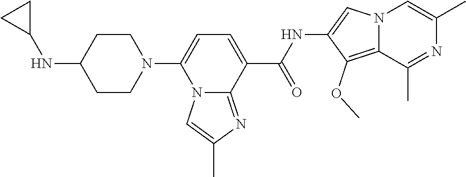 [SMILES: COc1c(NC(=O)c2ccc(N3CCC(NC4CC4)CC3)n3cc(C)nc23)cn2cc(C)nc(C)c12] |
| 632 | 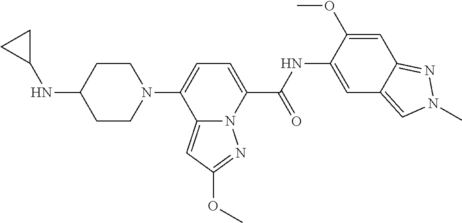 [SMILES: COc1cc2c(N3CCC(NC4CC4)CC3)ccc(C(=O)Nc3cc4cn(C)nc4cc3OC)n2n1] |
| 633 | 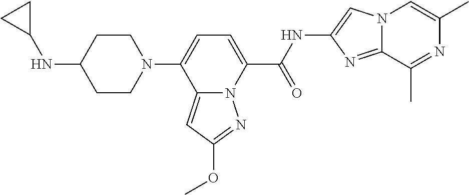 [SMILES: COc1cc2c(N3CCC(NC4CC4)CC3)ccc(C(=O)Nc3cn4cc(C)nc(C)c4n3)n2n1] |
| 634 | 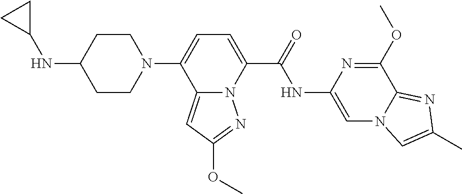 [SMILES: COc1cc2c(N3CCC(NC4CC4)CC3)ccc(C(=O)Nc3cn4cc(C)nc4c(OC)n3)n2n1] |
| 635 | 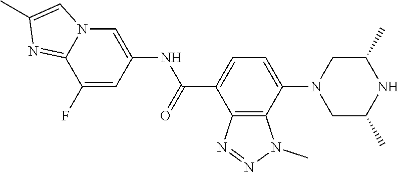 |
| 636 | 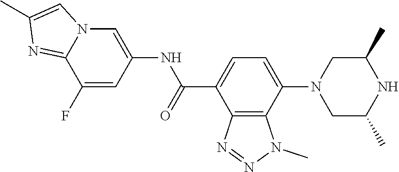 |
| 637 | 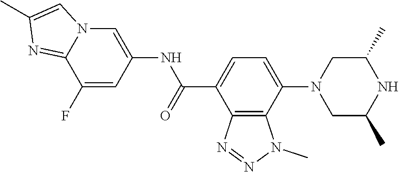 |
| 638 | 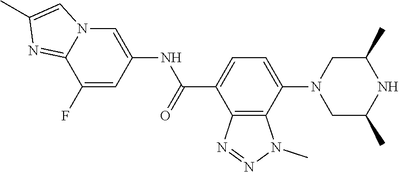 |
| 639 | 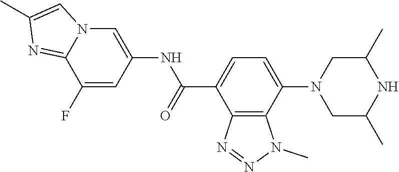 |
| 640 | 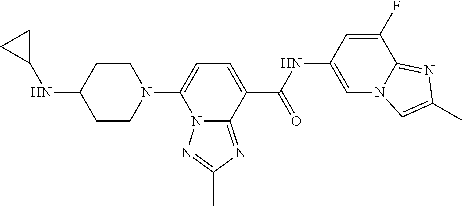 [SMILES: Cc1cn2cc(NC(=O)c3ccc(N4CCC(NC5CC5)CC4)n4nc(C)nc34)cc(F)c2n1] |
| 641 | 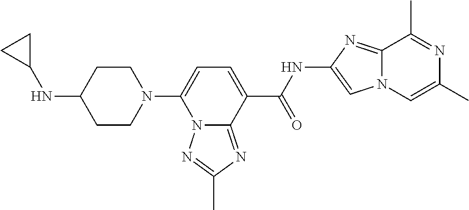 [SMILES: Cc1cn2cc(NC(=O)c3ccc(N4CCC(NC5CC5)CC4)n4nc(C)nc34)nc2c(C)n1] |
| 642 | 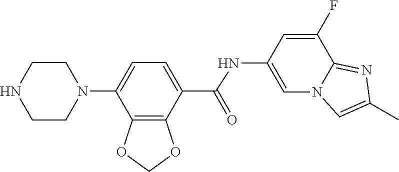 [SMILES: Cc1cn2cc(NC(=O)c3ccc(N4CCNCC4)c4c3OCO4)cc(F)c2n1] |
| 643 | 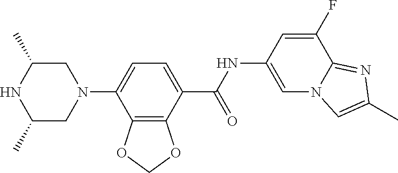 [SMILES: Cc1cn2cc(NC(=O)c3ccc(N4C[C@@H](C)N[C@@H](C)C4)c4c3OCO4)cc(F)c2n1] |
| 644 | 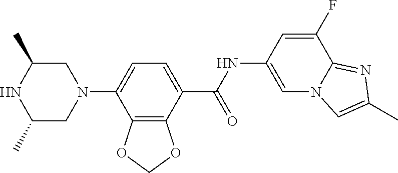 [SMILES: Cc1cn2cc(NC(=O)c3ccc(N4C[C@H](C)N[C@@H](C)C4)c4c3OCO4)cc(F)c2n1] |
| 645 | 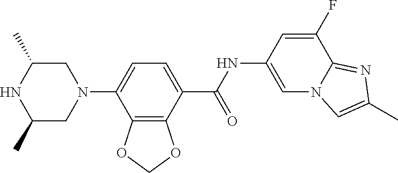 [SMILES: Cc1cn2cc(NC(=O)c3ccc(N4C[C@@H](C)N[C@H](C)C4)c4c3OCO4)cc(F)c2n1] |
| 646 | 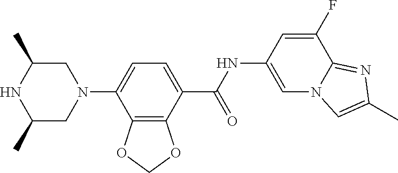 [SMILES: Cc1cn2cc(NC(=O)c3ccc(N4C[C@@H](C)N[C@@H](C)C4)c4c3OCO4)cc(F)c2n1] |
| 647 | 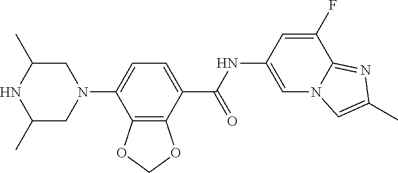 [SMILES: Cc1cn2cc(NC(=O)c3ccc(N4CC(C)NC(C)C4)c4c3OCO4)cc(F)c2n1] |
| 648 | 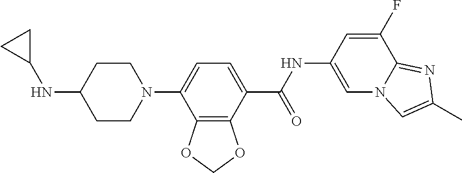 [SMILES: Cc1cn2cc(NC(=O)c3ccc(N4CCC(NC5CC5)CC4)c4c3OCO4)cc(F)c2n1] |
| 649 | 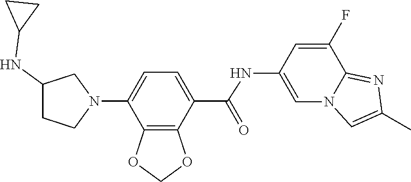 [SMILES: Cc1cn2cc(NC(=O)c3ccc(N4CCC(NC5CC5)C4)c4c3OCO4)cc(F)c2n1] |
| 650 | 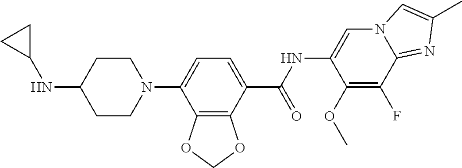 [SMILES: COc1c(NC(=O)c2ccc(N3CCC(NC4CC4)CC3)c3c2OCO3)cn2cc(C)nc2c1F] |
| 651 | 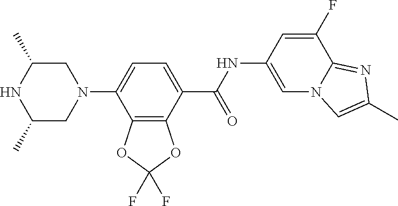 [SMILES: Cc1cn2cc(NC(=O)c3ccc(N4C[C@@H](C)N[C@@H](C)C4)c4c3OC(F)(F)O4)cc(F)c2n1] |
| 652 | 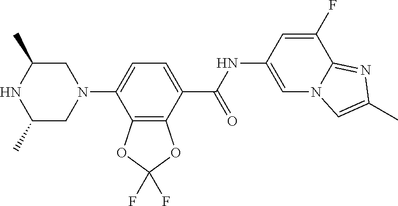 [SMILES: Cc1cn2cc(NC(=O)c3ccc(N4C[C@H](C)N[C@@H](C)C4)c4c3OC(F)(F)O4)cc(F)c2n1] |
| 653 | 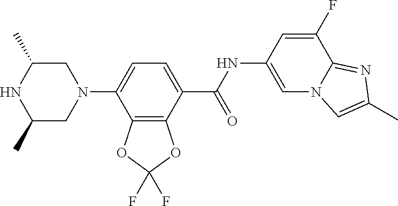 [SMILES: Cc1cn2cc(NC(=O)c3ccc(N4C[C@@H](C)N[C@H](C)C4)c4c3OC(F)(F)O4)cc(F)c2n1] |
| 654 | 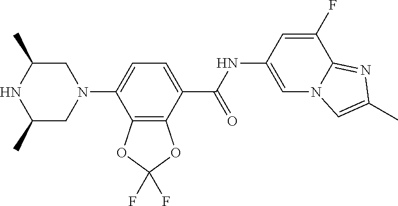 [SMILES: Cc1cn2cc(NC(=O)c3ccc(N4C[C@@H](C)N[C@@H](C)C4)c4c3OC(F)(F)O4)cc(F)c2n1] |
| 655 | 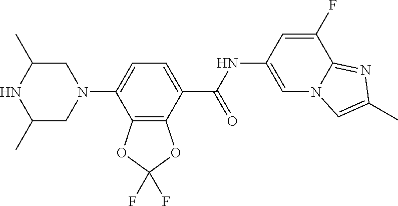 [SMILES: Cc1cn2cc(NC(=O)c3ccc(N4CC(C)NC(C)C4)c4c3OC(F)(F)O4)cc(F)c2n1] |
| 656 | 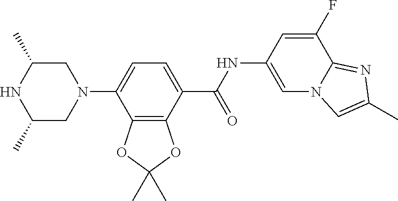 [SMILES: Cc1cn2cc(NC(=O)c3ccc(N4C[C@@H](C)N[C@@H](C)C4)c4c3OC(C)(C)O4)cc(F)c2n1] |
| 657 | 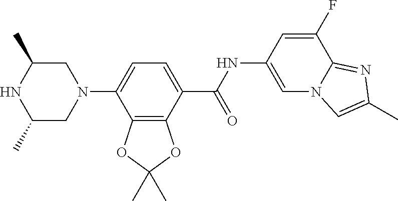 [SMILES: Cc1cn2cc(NC(=O)c3ccc(N4C[C@H](C)N[C@@H](C)C4)c4c3OC(C)(C)O4)cc(F)c2n1] |
| 658 | 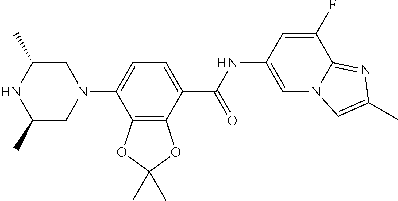 [SMILES: Cc1cn2cc(NC(=O)c3ccc(N4C[C@@H](C)N[C@H](C)C4)c4c3OC(C)(C)O4)cc(F)c2n1] |
| 659 | 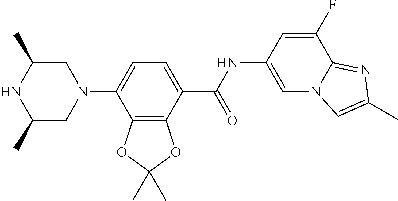 [SMILES: Cc1cn2cc(NC(=O)c3ccc(N4C[C@@H](C)N[C@@H](C)C4)c4c3OC(C)(C)O4)cc(F)c2n1] |
| 660 | 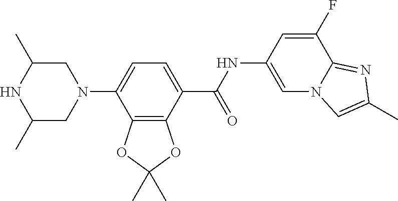 [SMILES: Cc1cn2cc(NC(=O)c3ccc(N4CC(C)NC(C)C4)c4c3OC(C)(C)O4)cc(F)c2n1] |
| 661 | 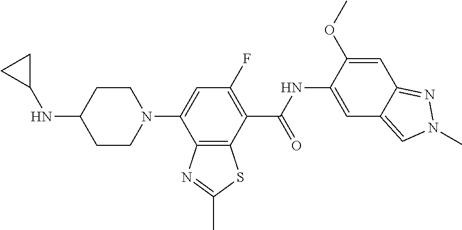 [SMILES: COc1cc2nn(C)cc2cc1NC(=O)c1c(F)cc(N2CCC(NC3CC3)CC2)c2nc(C)sc12] |
| 662 | 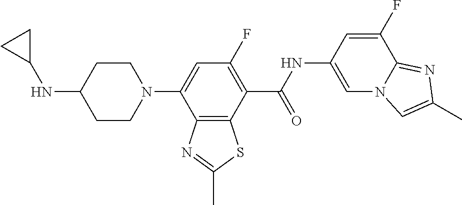 [SMILES: Cc1cn2cc(NC(=O)c3c(F)cc(N4CCC(NC5CC5)CC4)c4nc(C)sc34)cc(F)c2n1] |
| 663 | 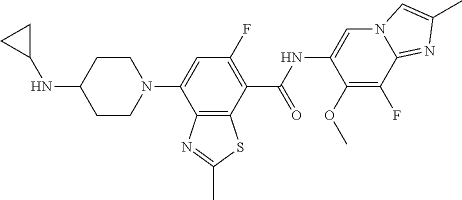 [SMILES: COc1c(NC(=O)c2c(F)cc(N3CCC(NC4CC4)CC3)c3nc(C)sc23)cn2cc(C)nc2c1F] |
| 664 | 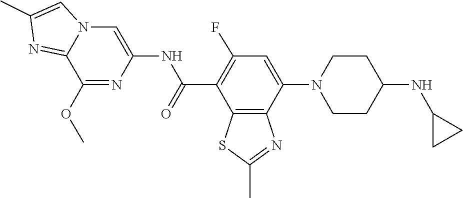 [SMILES: COc1nc(NC(=O)c2c(F)cc(N3CCC(NC4CC4)CC3)c3nc(C)sc23)cn2cc(C)nc12] |
| 665 | 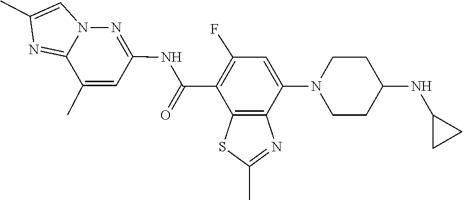 [SMILES: Cc1cn2nc(NC(=O)c3c(F)cc(N4CCC(NC5CC5)CC4)c4nc(C)sc34)cc(C)c2n1] |
| 666 | 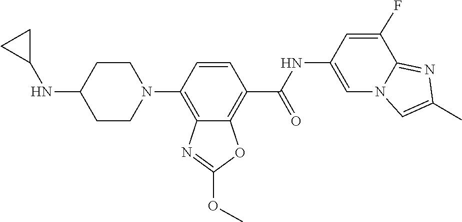 [SMILES: COc1nc2c(N3CCC(NC4CC4)CC3)ccc(C(=O)Nc3cc(F)c4nc(C)cn4c3)c2o1] |
| 667 | 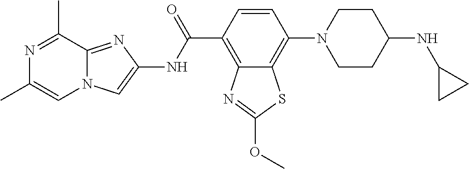 [SMILES: COc1nc2c(C(=O)Nc3cn4cc(C)nc(C)c4n3)ccc(N3CCC(NC4CC4)CC3)c2s1] |
| 668 | 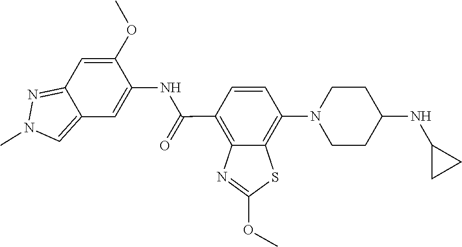 [SMILES: COc1nc2c(C(=O)Nc3cc4cn(C)nc4cc3OC)ccc(N3CCC(NC4CC4)CC3)c2s1] |
| 669 | 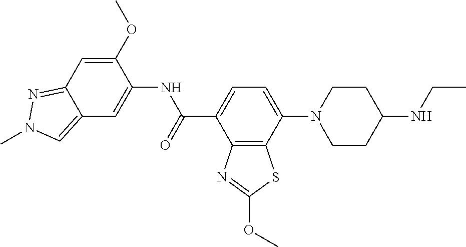 [SMILES: CCNC1CCN(c2ccc(C(=O)Nc3cc4cn(C)nc4cc3OC)c3nc(OC)sc23)CC1] |
| 670 | 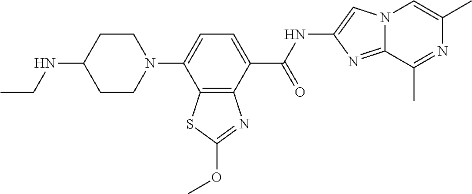 [SMILES: CCNC1CCN(c2ccc(C(=O)Nc3cn4cc(C)nc(C)c4n3)c3nc(OC)sc23)CC1] |
| 671 | 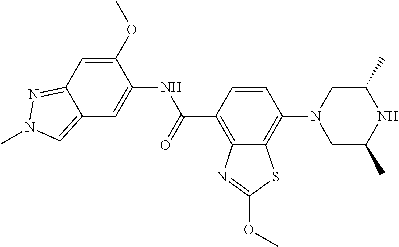 |
| 672 | 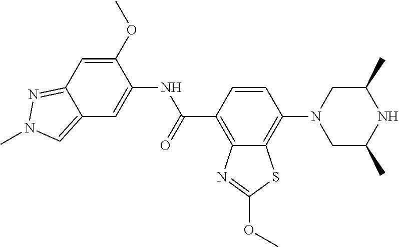 |
| 673 | 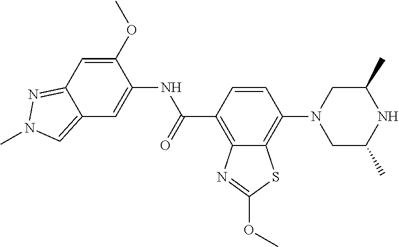 |
| 674 | 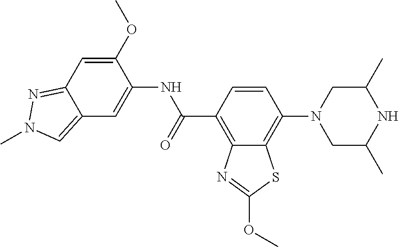 |
| 675 | 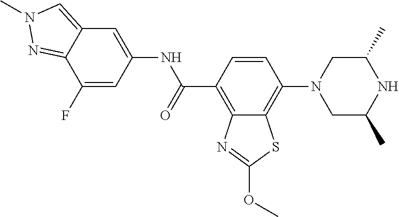 |
| 676 | 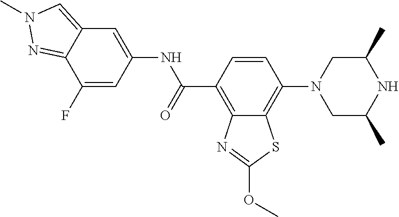 |
| 677 | 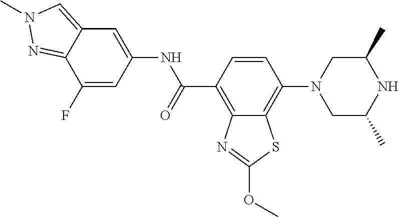 |
| 678 | 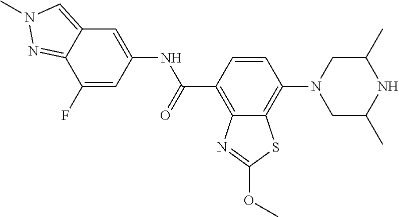 |
| 679 | 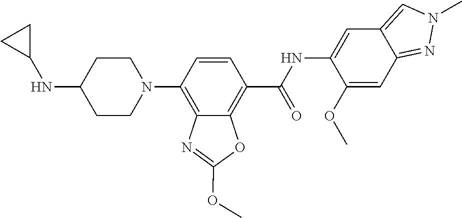 [SMILES: COc1nc2c(N3CCC(NC4CC4)CC3)ccc(C(=O)Nc3cc4cn(C)nc4cc3OC)c2o1] |
| 680 | 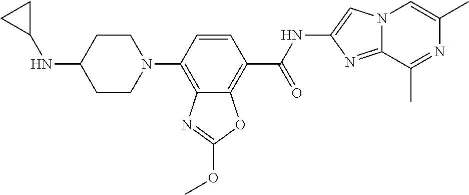 [SMILES: COc1nc2c(N3CCC(NC4CC4)CC3)ccc(C(=O)Nc3cn4cc(C)nc(C)c4n3)c2o1] |
| 681 | 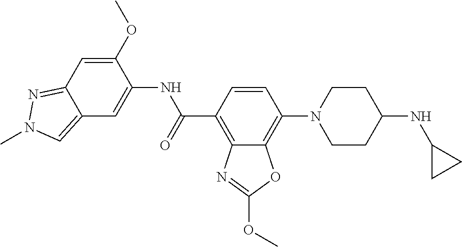 [SMILES: COc1nc2c(C(=O)Nc3cc4cn(C)nc4cc3OC)ccc(N3CCC(NC4CC4)CC3)c2o1] |
| 682 | 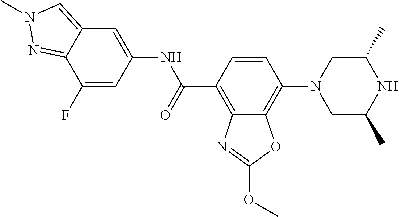 |
| 683 | 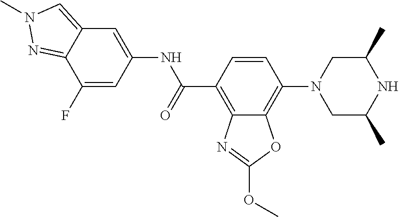 |
| 684 | 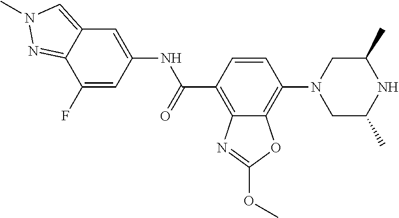 |
| 685 | 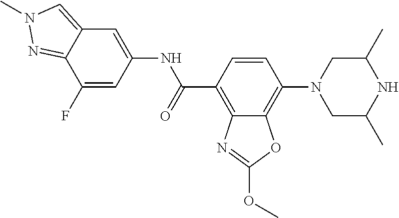 |
| 686 | 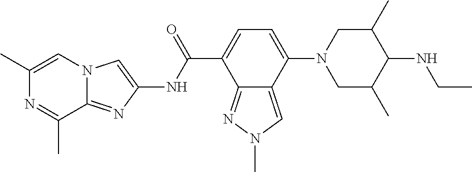 [SMILES: CCNC1C(C)CN(c2ccc(C(=O)Nc3cn4cc(C)nc(C)c4n3)c3nn(C)cc23)CC1C] |
| 687 | 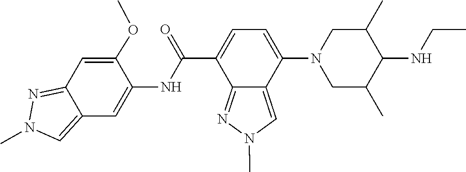 [SMILES: CCNC1C(C)CN(c2ccc(C(=O)Nc3cc4cn(C)nc4cc3OC)c3nn(C)cc23)CC1C] |
| 688 | 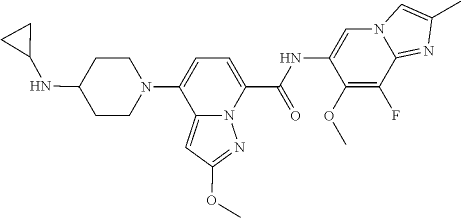 [SMILES: COc1cc2c(N3CCC(NC4CC4)CC3)ccc(C(=O)Nc3cn4cc(C)nc4c(F)c3OC)n2n1] |
| 689 | 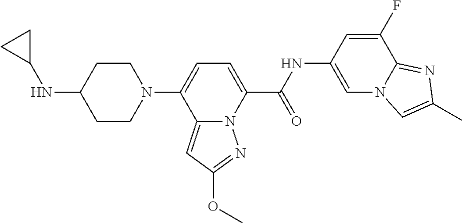 [SMILES: COc1cc2c(N3CCC(NC4CC4)CC3)ccc(C(=O)Nc3cc(F)c4nc(C)cn4c3)n2n1] |
| 690 | 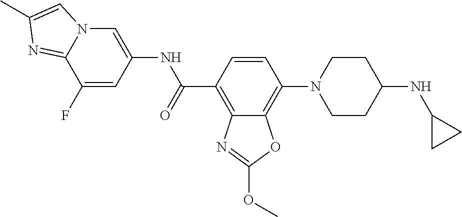 [SMILES: COc1nc2c(C(=O)Nc3cc(F)c4nc(C)cn4c3)ccc(N3CCC(NC4CC4)CC3)c2o1] |
| 691 | 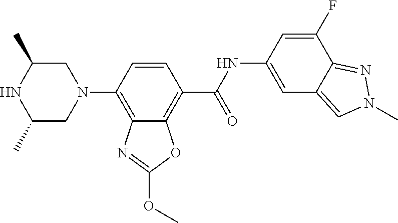 [SMILES: COc1nc2c(N3C[C@H](C)N[C@@H](C)C3)ccc(C(=O)Nc3cc(F)c4nn(C)cc4c3)c2o1] |
| 692 | 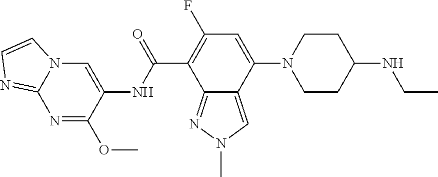 [SMILES: CCNC1CCN(c2cc(F)c(C(=O)Nc3cn4ccnc4nc3OC)c3nn(C)cc23)CC1] |
| 694 | 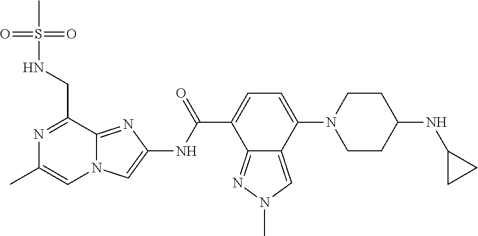 [SMILES: Cc1cn2cc(NC(=O)c3ccc(N4CCC(NC5CC5)CC4)c4cn(C)nc34)nc2c(CNS(C)(=O)=O)n1] |
| 695 | 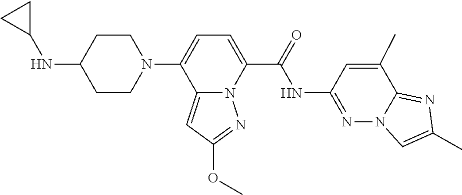 [SMILES: COc1cc2c(N3CCC(NC4CC4)CC3)ccc(C(=O)Nc3cc(C)c4nc(C)cn4n3)n2n1] |
| 696 | 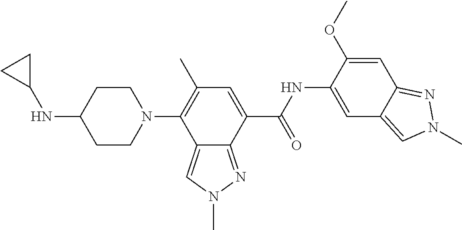 [SMILES: COc1cc2nn(C)cc2cc1NC(=O)c1cc(C)c(N2CCC(NC3CC3)CC2)c2cn(C)nc12] |
| 697 | 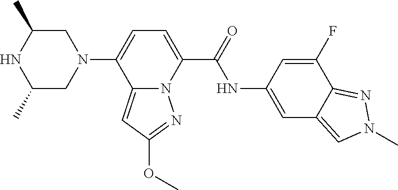 [SMILES: COc1cc2c(N3C[C@H](C)N[C@@H](C)C3)ccc(C(=O)Nc3cc(F)c4nn(C)cc4c3)n2n1] |
| 698 | 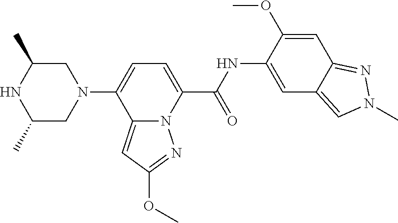 [SMILES: COc1cc2c(N3C[C@H](C)N[C@@H](C)C3)ccc(C(=O)Nc3cc4cn(C)nc4cc3OC)n2n1] |
| 699 | 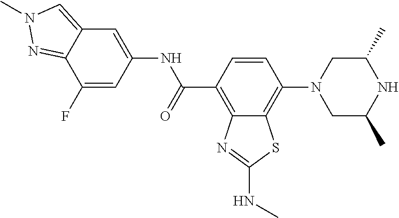 [SMILES: CNc1nc2c(C(=O)Nc3cc(F)c4nn(C)cc4c3)ccc(N3C[C@H](C)N[C@@H](C)C3)c2s1] |
| 700 | 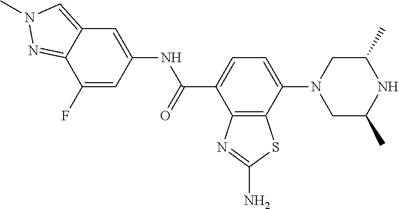 |
| 701 | 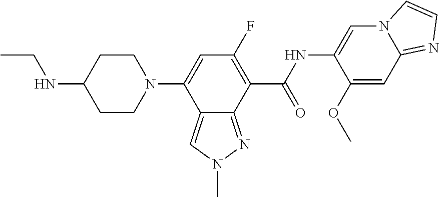 [SMILES: CCNC1CCN(c2cc(F)c(C(=O)Nc3cn4ccnc4cc3OC)c3nn(C)cc23)CC1] |
| 702 | 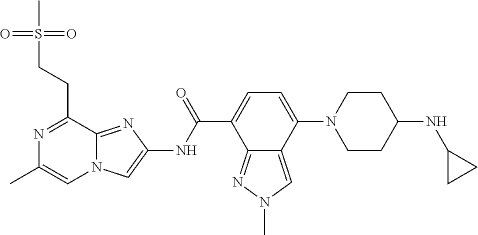 [SMILES: Cc1cn2cc(NC(=O)c3ccc(N4CCC(NC5CC5)CC4)c4cn(C)nc34)nc2c(CCS(C)(=O)=O)n1] |
| 703 | 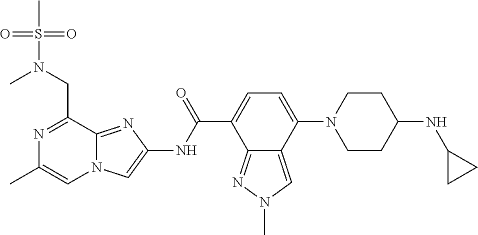 [SMILES: Cc1cn2cc(NC(=O)c3ccc(N4CCC(NC5CC5)CC4)c4cn(C)nc34)nc2c(CN(C)S(C)(=O)=O)n1] |
| 704 | 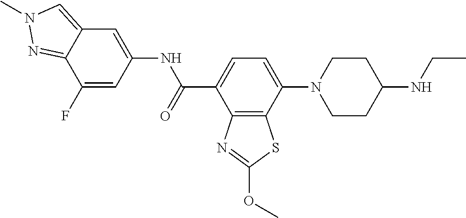 [SMILES: CCNC1CCN(c2ccc(C(=O)Nc3cc(F)c4nn(C)cc4c3)c3nc(OC)sc23)CC1] |
| 705 | 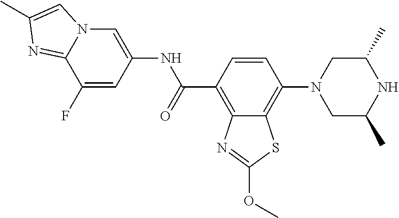 |
| 706 | 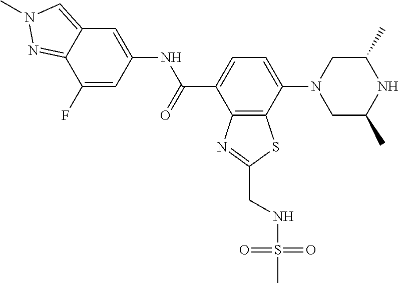 |
| 707 | 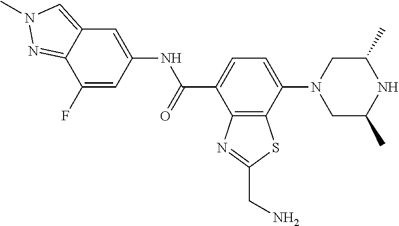 |
| 708 | 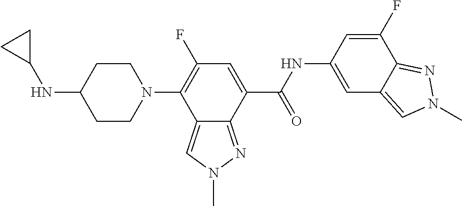 [SMILES: Cn1cc2cc(NC(=O)c3cc(F)c(N4CCC(NC5CC5)CC4)c4cn(C)nc34)cc(F)c2n1] |
| 709 | 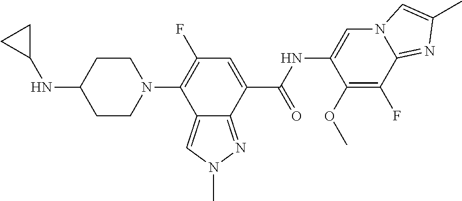 [SMILES: COc1c(NC(=O)c2cc(F)c(N3CCC(NC4CC4)CC3)c3cn(C)nc23)cn2cc(C)nc2c1F] |
| 710 | 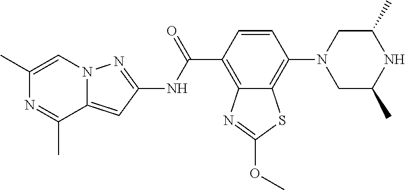 |
| 711 | 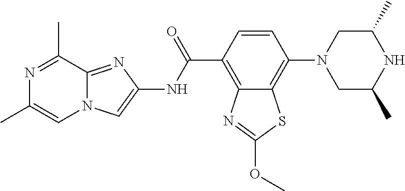 |
| 712 | 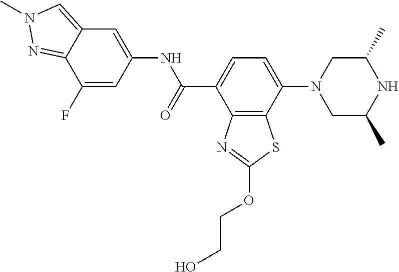 |
| 713 | 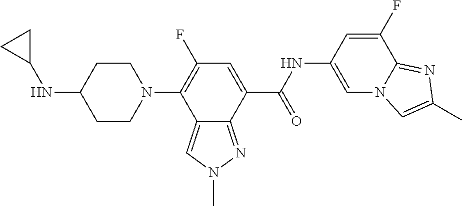 [SMILES: Cc1cn2cc(NC(=O)c3cc(F)c(N4CCC(NC5CC5)CC4)c4cn(C)nc34)cc(F)c2n1] |
| 714 | 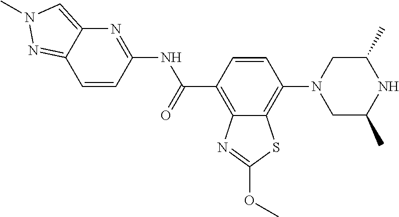 [SMILES: COc1nc2c(C(=O)Nc3ccc4nn(C)cc4n3)ccc(N3C[C@H](C)N[C@@H](C)C3)c2s1] |
| 715 | 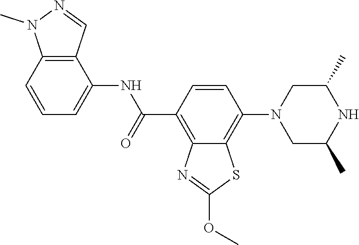 |
| 716 | 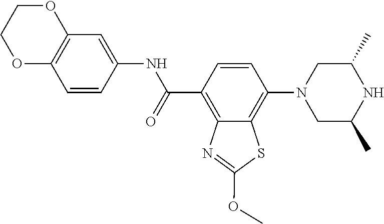 [SMILES: COc1nc2c(C(=O)Nc3ccc4c(c3)OCCO4)ccc(N3C[C@H](C)N[C@@H](C)C3)c2s1] |
| 717 | 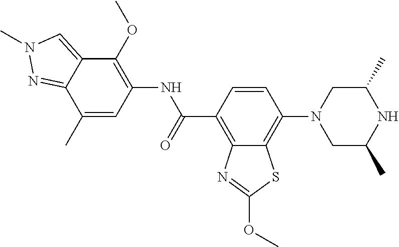 |
| 718 | 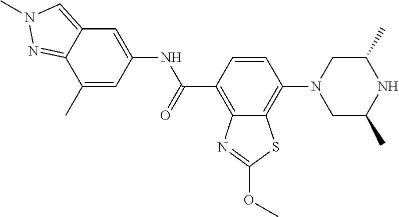 |
| 719 | 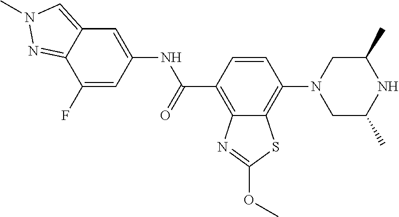 |
| 720 | 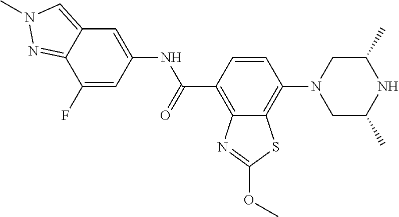 |
| 721 | 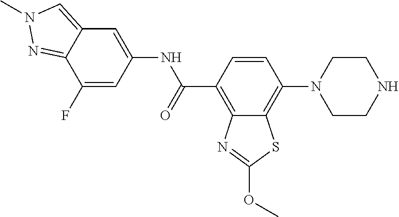 [SMILES: COc1nc2c(C(=O)Nc3cc(F)c4nn(C)cc4c3)ccc(N3CCNCC3)c2s1] |
| 722 | 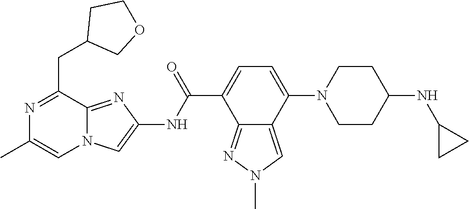 [SMILES: Cc1cn2cc(NC(=O)c3ccc(N4CCC(NC5CC5)CC4)c4cn(C)nc34)nc2c(CC2CCOC2)n1] |
| 723 | 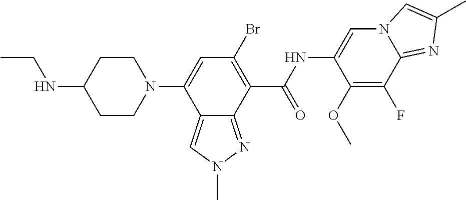 [SMILES: CCNC1CCN(c2cc(Br)c(C(=O)Nc3cn4cc(C)nc4c(F)c3OC)c3nn(C)cc23)CC1] |
| 724 | 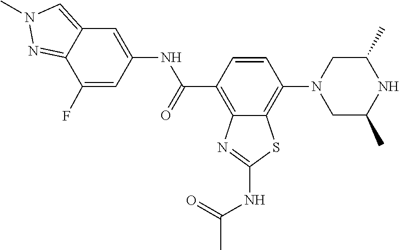 [SMILES: CC(=O)Nc1nc2c(C(=O)Nc3cc(F)c4nn(C)cc4c3)ccc(N3C[C@H](C)N[C@@H](C)C3)c2s1] |
| 725 | 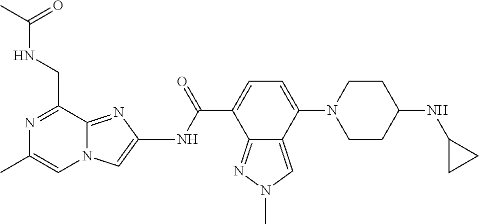 [SMILES: CC(=O)NCc1nc(C)cn2cc(NC(=O)c3ccc(N4CCC(NC5CC5)CC4)c4cn(C)nc34)nc12] |
| 726 | 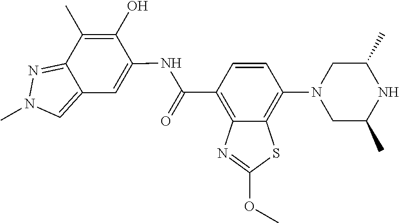 [SMILES: COc1nc2c(C(=O)Nc3cc4cn(C)nc4c(C)c3O)ccc(N3C[C@H](C)N[C@@H](C)C3)c2s1] |
| 727 | 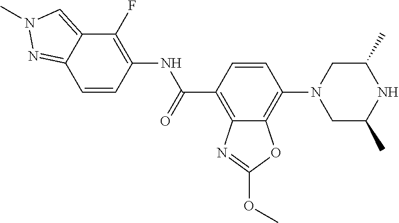 |
| 728 | 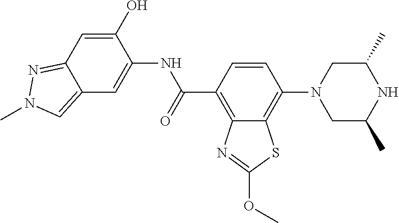 |
| 729 | 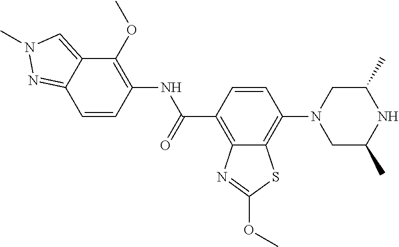 |
| 730 | 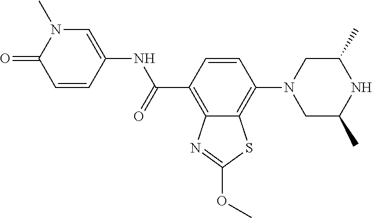 [SMILES: COc1nc2c(C(=O)Nc3ccc(=O)n(C)c3)ccc(N3C[C@H](C)N[C@@H](C)C3)c2s1] |
| 731 | 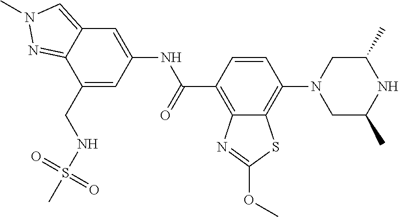 |
| 732 | 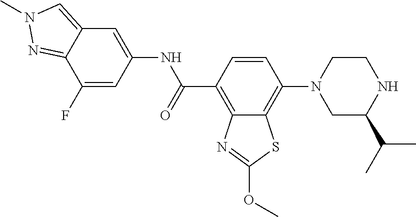 [SMILES: COc1nc2c(C(=O)Nc3cc(F)c4nn(C)cc4c3)ccc(N3CCN[C@@H](C(C)C)C3)c2s1] |
| 733 | 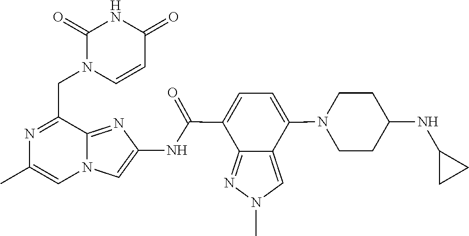 [SMILES: Cc1cn2cc(NC(=O)c3ccc(N4CCC(NC5CC5)CC4)c4cn(C)nc34)nc2c(Cn2ccc(=O)[nH]c2=O)n1] |
| 734 | 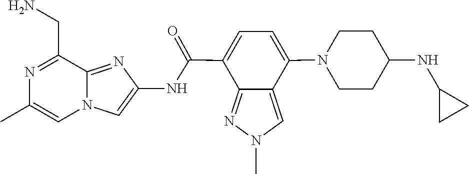 [SMILES: Cc1cn2cc(NC(=O)c3ccc(N4CCC(NC5CC5)CC4)c4cn(C)nc34)nc2c(CN)n1] |
| 735 | 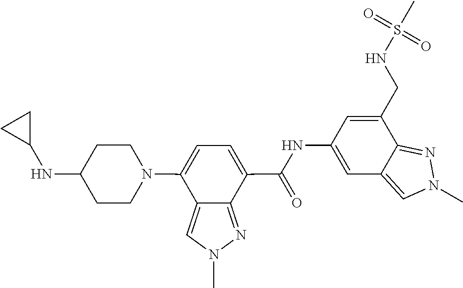 [SMILES: Cn1cc2cc(NC(=O)c3ccc(N4CCC(NC5CC5)CC4)c4cn(C)nc34)cc(CNS(C)(=O)=O)c2n1] |
| 736 | 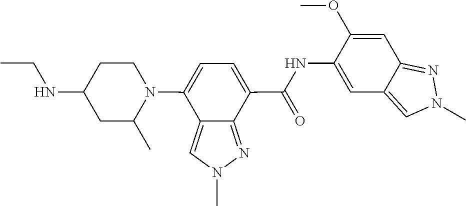 [SMILES: CCNC1CCN(c2ccc(C(=O)Nc3cc4cn(C)nc4cc3OC)c3nn(C)cc23)C(C)C1] |
| 737 | 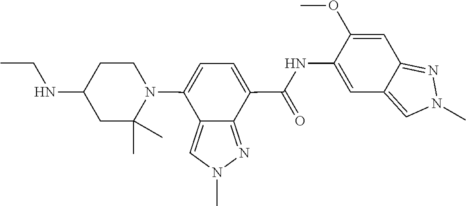 [SMILES: CCNC1CCN(c2ccc(C(=O)Nc3cc4cn(C)nc4cc3OC)c3nn(C)cc23)C(C)(C)C1] |
| 738 | 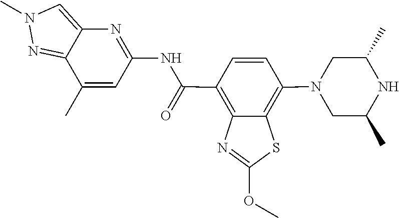 |
| 739 | 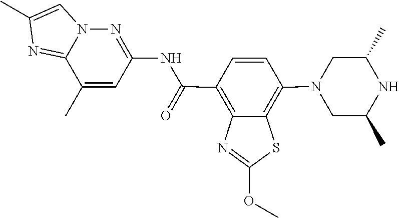 |
| 740 | 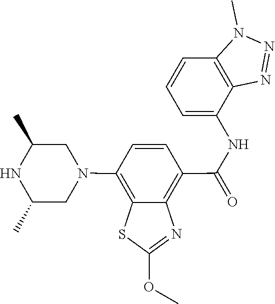 [SMILES: COc1nc2c(C(=O)Nc3cccc4c3nnn4C)ccc(N3C[C@H](C)N[C@@H](C)C3)c2s1] |
| 741 | 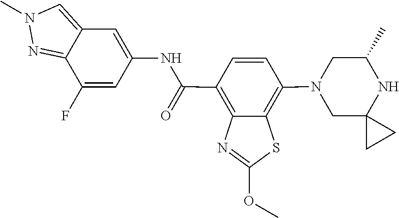 [SMILES: COc1nc2c(C(=O)Nc3cc(F)c4nn(C)cc4c3)ccc(N3C[C@H](C)NC4(CC4)C3)c2s1] |
| 742 | 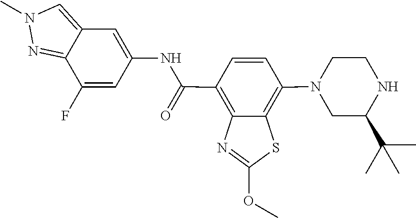 [SMILES: COc1nc2c(C(=O)Nc3cc(F)c4nn(C)cc4c3)ccc(N3CCN[C@@H](C(C)(C)C)C3)c2s1] |
| 743 | 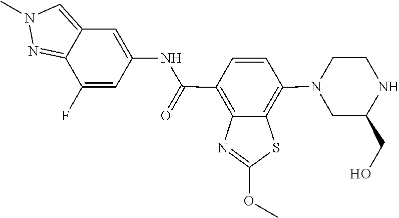 [SMILES: COc1nc2c(C(=O)Nc3cc(F)c4nn(C)cc4c3)ccc(N3CCN[C@@H](CO)C3)c2s1] |
| 744 | 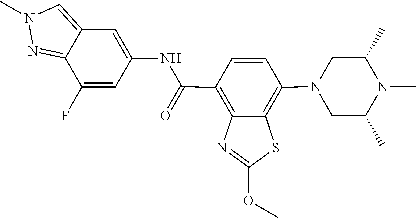 |
| 745 | 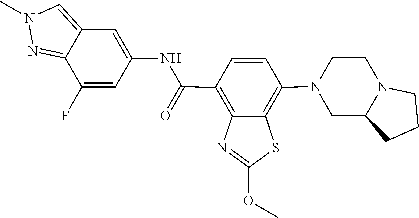 [SMILES: COc1nc2c(C(=O)Nc3cc(F)c4nn(C)cc4c3)ccc(N3CCN4CCC[C@H]4C3)c2s1] |
| 746 | 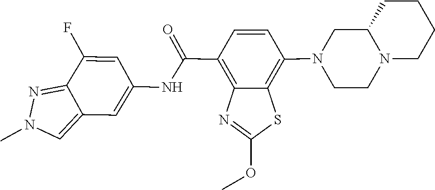 [SMILES: COc1nc2c(C(=O)Nc3cc(F)c4nn(C)cc4c3)ccc(N3CCN4CCCC[C@H]4C3)c2s1] |
| 747 | 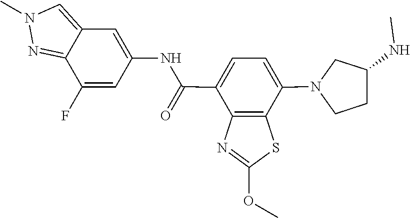 [SMILES: CN[C@@H]1CCN(c2ccc(C(=O)Nc3cc(F)c4nn(C)cc4c3)c3nc(OC)sc23)C1] |
| 748 | 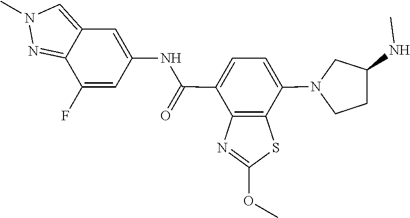 [SMILES: CN[C@H]1CCN(c2ccc(C(=O)Nc3cc(F)c4nn(C)cc4c3)c3nc(OC)sc23)C1] |
| 749 | 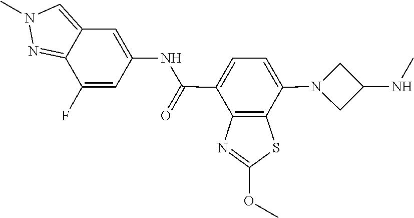 |
| 750 | 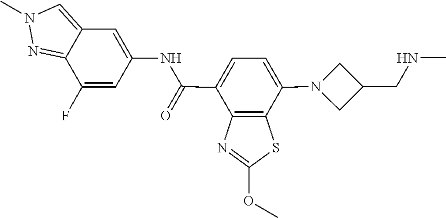 |
| 752 | 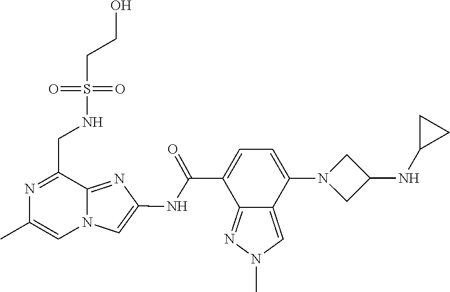 [SMILES: Cc1cn2cc(NC(=O)c3ccc(N4CC(NC5CC5)C4)c4cn(C)nc34)nc2c(CNS(=O)(=O)CCO)n1] |
| 753 | 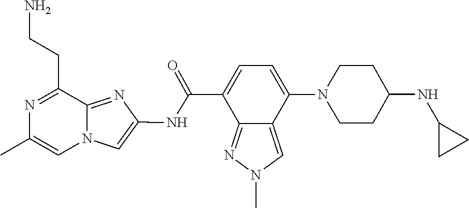 [SMILES: Cc1cn2cc(NC(=O)c3ccc(N4CCC(NC5CC5)CC4)c4cn(C)nc34)nc2c(CCN)n1] |
| 754 | 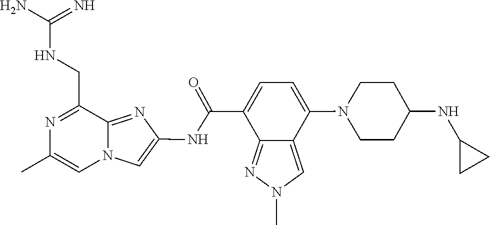 [SMILES: Cc1cn2cc(NC(=O)c3ccc(N4CCC(NC5CC5)CC4)c4cn(C)nc34)nc2c(CNC(=N)N)n1] |
| 755 | 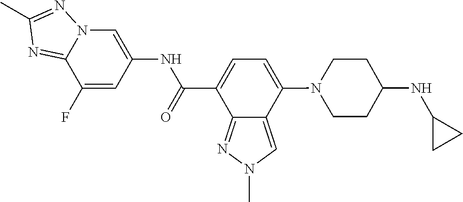 [SMILES: Cc1nc2c(F)cc(NC(=O)c3ccc(N4CCC(NC5CC5)CC4)c4cn(C)nc34)cn2n1] |
| 756 | 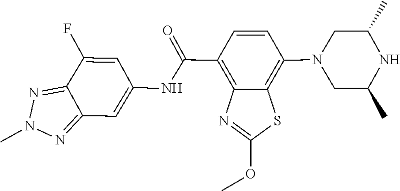 |
| 757 | 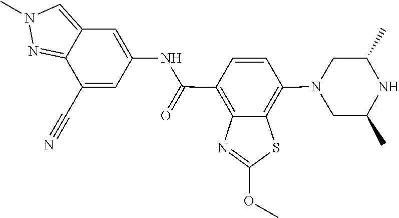 |
| 758 | 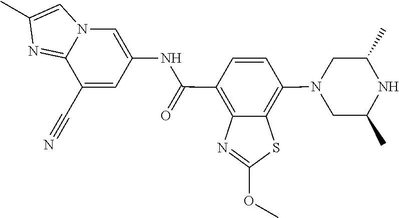 |
| 759 | 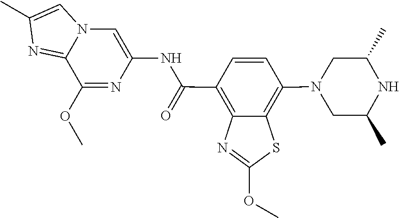 |
| 760 | 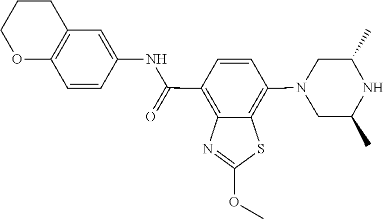 [SMILES: COc1nc2c(C(=O)Nc3ccc4c(c3)CCCO4)ccc(N3C[C@H](C)N[C@@H](C)C3)c2s1] |
| 761 | 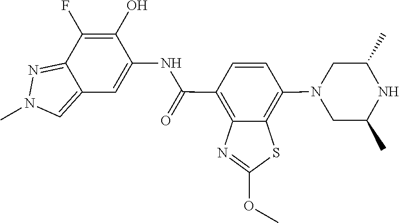 |
| 762 | 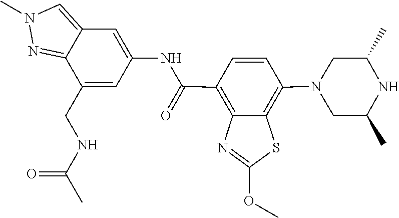 |
| 763 | 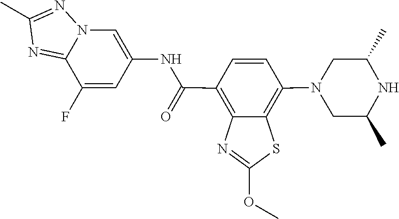 |
| 764 | 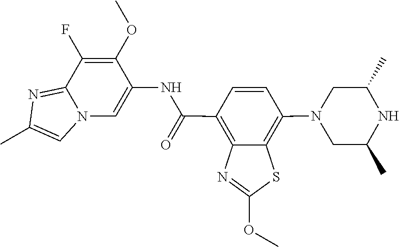 |
| 765 | 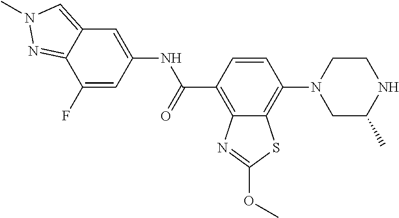 |
| 766 | 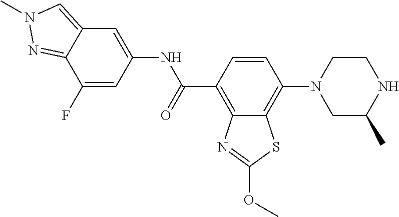 |
| 767 | 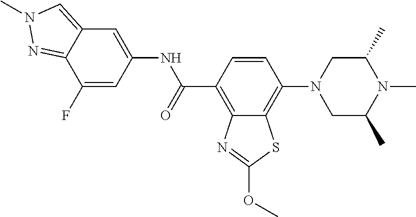 |
| 768 | 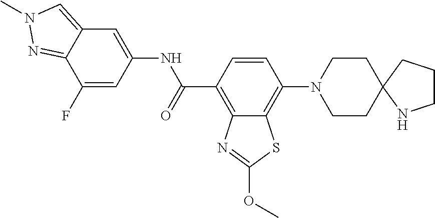 [SMILES: COc1nc2c(C(=O)Nc3cc(F)c4nn(C)cc4c3)ccc(N3CCC4(CCCN4)CC3)c2s1] |
| 769 | 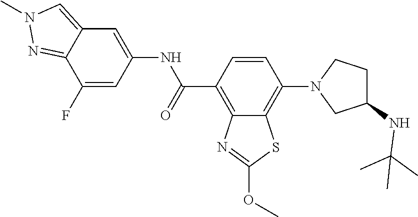 [SMILES: COc1nc2c(C(=O)Nc3cc(F)c4nn(C)cc4c3)ccc(N3CC[C@@H](NC(C)(C)C)C3)c2s1] |
| 770 | 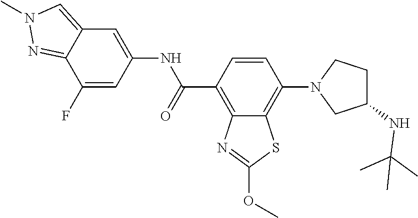 [SMILES: COc1nc2c(C(=O)Nc3cc(F)c4nn(C)cc4c3)ccc(N3CC[C@H](NC(C)(C)C)C3)c2s1] |
| 771 | 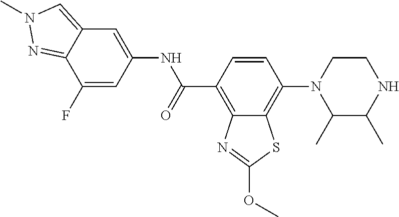 |
| 772 | 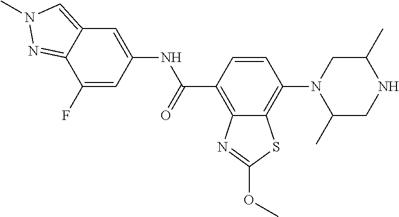 |
| 773 | 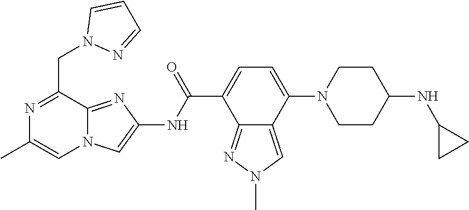 [SMILES: Cc1cn2cc(NC(=O)c3ccc(N4CCC(NC5CC5)CC4)c4cn(C)nc34)nc2c(Cn2cccn2)n1] |
| 774 | 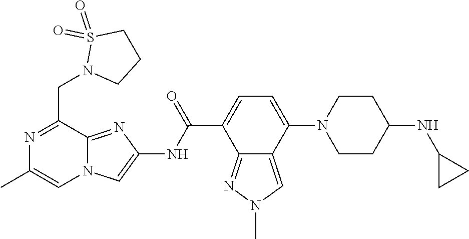 [SMILES: Cc1cn2cc(NC(=O)c3ccc(N4CCC(NC5CC5)CC4)c4cn(C)nc34)nc2c(CN2CCCS2(=O)=O)n1] |
| 775 | 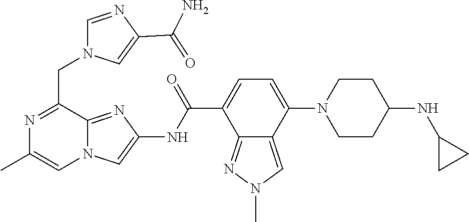 [SMILES: Cc1cn2cc(NC(=O)c3ccc(N4CCC(NC5CC5)CC4)c4cn(C)nc34)nc2c(Cn2cnc(C(N)=O)c2)n1] |
| 776 | 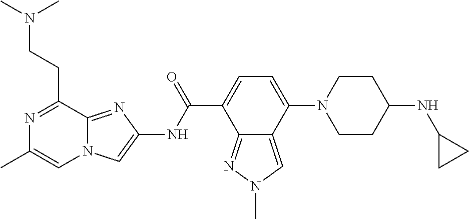 [SMILES: Cc1cn2cc(NC(=O)c3ccc(N4CCC(NC5CC5)CC4)c4cn(C)nc34)nc2c(CCN(C)C)n1] |
| 777 | 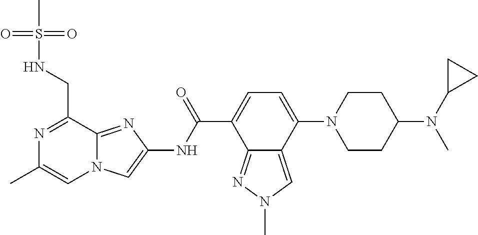 [SMILES: Cc1cn2cc(NC(=O)c3ccc(N4CCC(N(C)C5CC5)CC4)c4cn(C)nc34)nc2c(CNS(C)(=O)=O)n1] |
| 778 | 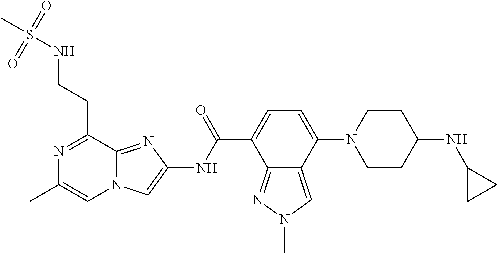 [SMILES: Cc1cn2cc(NC(=O)c3ccc(N4CCC(NC5CC5)CC4)c4cn(C)nc34)nc2c(CCNS(C)(=O)=O)n1] |
| 779 | 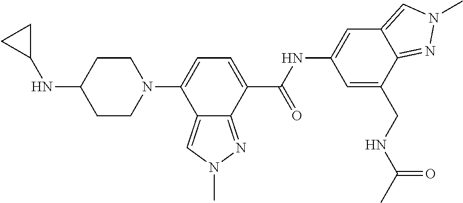 [SMILES: CC(=O)NCc1cc(NC(=O)c2ccc(N3CCC(NC4CC4)CC3)c3cn(C)nc23)cc2cn(C)nc12] |
| 780 | 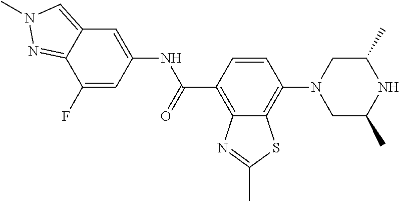 |
| 781 | 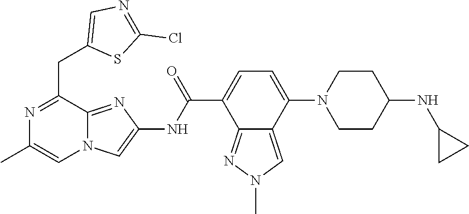 [SMILES: Cc1cn2cc(NC(=O)c3ccc(N4CCC(NC5CC5)CC4)c4cn(C)nc34)nc2c(Cc2cnc(Cl)s2)n1] |
| 782 | 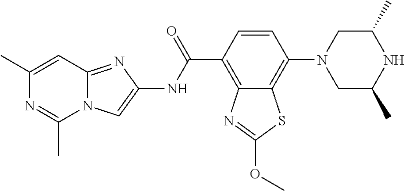 |
| 783 | 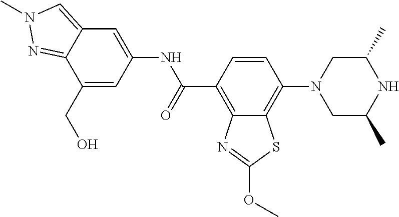 |
| 784 | 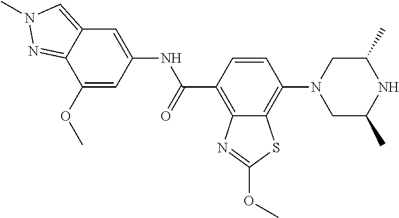 |
| 785 | 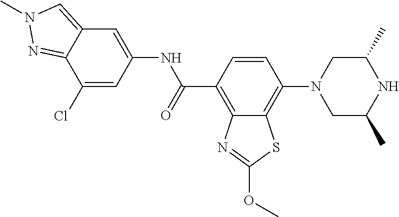 [SMILES: COc1nc2c(C(=O)Nc3cc(Cl)c4nn(C)cc4c3)ccc(N3C[C@H](C)N[C@@H](C)C3)c2s1] |
| 786 | 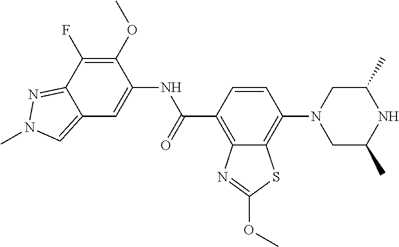 |
| 787 | 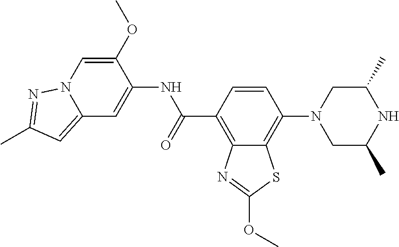 |
| 788 | 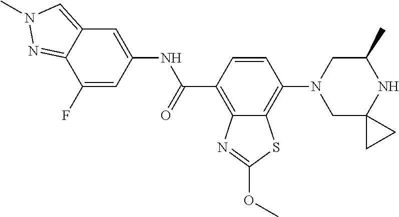 [SMILES: COc1nc2c(C(=O)Nc3cc(F)c4nn(C)cc4c3)ccc(N3C[C@@H](C)NC4(CC4)C3)c2s1] |
| 789 | 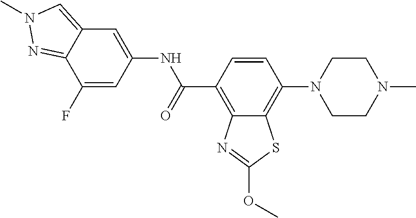 |
| 790 | 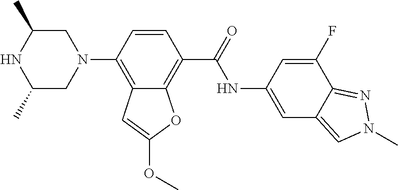 |
| 791 | 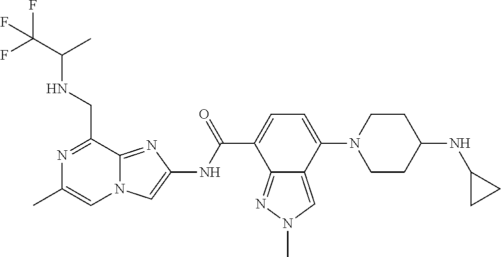 [SMILES: Cc1cn2cc(NC(=O)c3ccc(N4CCC(NC5CC5)CC4)c4cn(C)nc34)nc2c(CNC(C)C(F)(F)F)n1] |
| 792 | 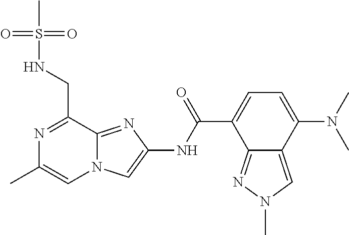 [SMILES: Cc1cn2cc(NC(=O)c3ccc(N(C)C)c4cn(C)nc34)nc2c(CNS(C)(=O)=O)n1] |
| 793 | 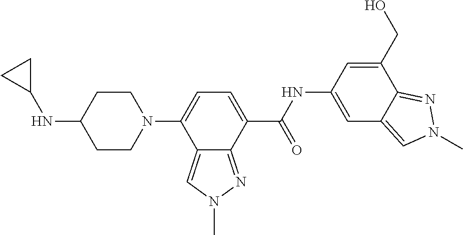 [SMILES: Cn1cc2cc(NC(=O)c3ccc(N4CCC(NC5CC5)CC4)c4cn(C)nc34)cc(CO)c2n1] |
| 794 | 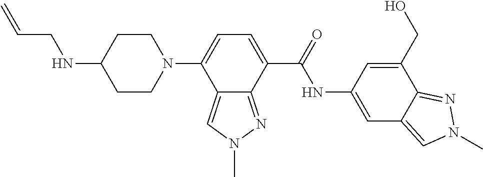 |
| 795 | 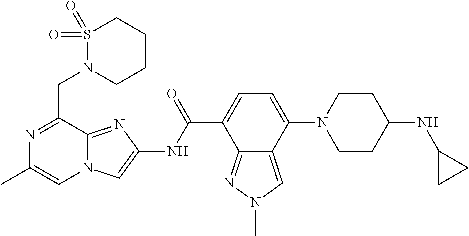 [SMILES: Cc1cn2cc(NC(=O)c3ccc(N4CCC(NC5CC5)CC4)c4cn(C)nc34)nc2c(CN2CCCCS2(=O)=O)n1] |
| 796 | 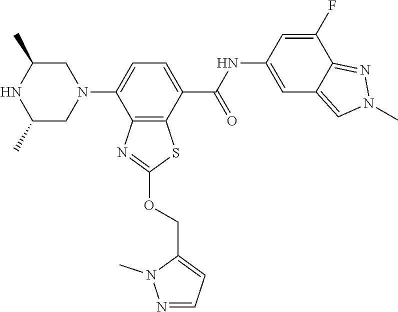 [SMILES: C[C@H]1CN(c2ccc(C(=O)Nc3cc(F)c4nn(C)cc4c3)c3sc(OCc4ccnn4C)nc23)C[C@H](C)N1] |
| 797 | 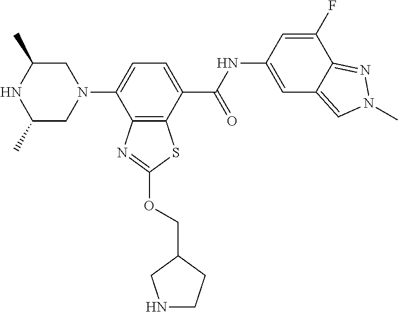 [SMILES: C[C@H]1CN(c2ccc(C(=O)Nc3cc(F)c4nn(C)cc4c3)c3sc(OCC4CCNC4)nc23)C[C@H](C)N1] |
| 798 | 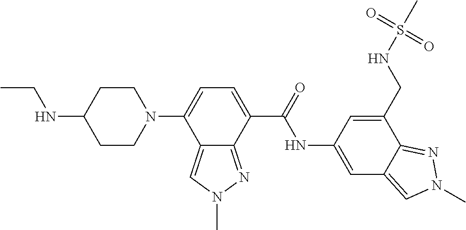 [SMILES: CCNC1CCN(c2ccc(C(=O)Nc3cc(CNS(C)(=O)=O)c4nn(C)cc4c3)c3nn(C)cc23)CC1] |
| 799 | 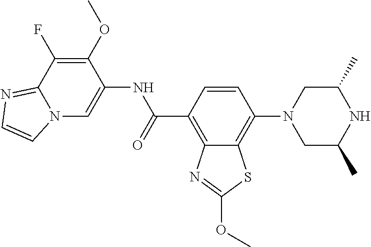 [SMILES: COc1nc2c(C(=O)Nc3cn4ccnc4c(F)c3OC)ccc(N3C[C@H](C)N[C@@H](C)C3)c2s1] |
| 800 | 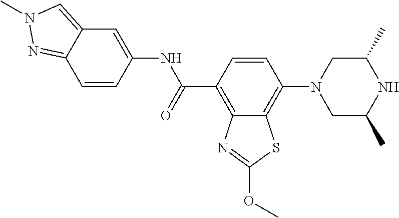 |
| 801 | 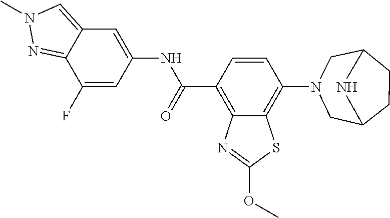 [SMILES: COc1nc2c(C(=O)Nc3cc(F)c4nn(C)cc4c3)ccc(N3CC4CCC(C3)N4)c2s1] |
| 803 | 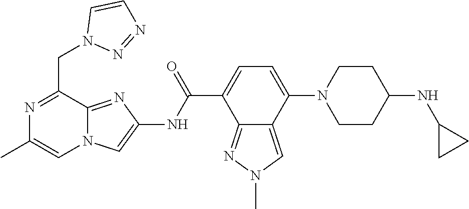 [SMILES: Cc1cn2cc(NC(=O)c3ccc(N4CCC(NC5CC5)CC4)c4cn(C)nc34)nc2c(Cn2ccnn2)n1] |
| 804 | 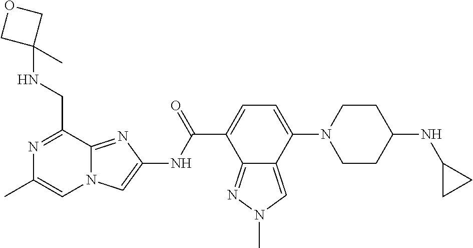 [SMILES: Cc1cn2cc(NC(=O)c3ccc(N4CCC(NC5CC5)CC4)c4cn(C)nc34)nc2c(CNC2(C)COC2)n1] |
| 805 | 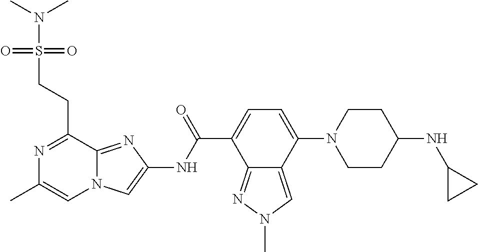 [SMILES: Cc1cn2cc(NC(=O)c3ccc(N4CCC(NC5CC5)CC4)c4cn(C)nc34)nc2c(CCS(=O)(=O)N(C)C)n1] |
| 806 | 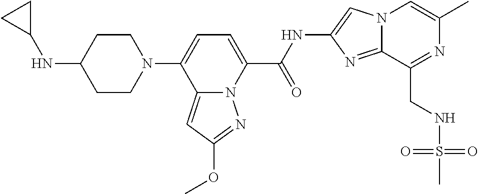 [SMILES: COc1cc2c(N3CCC(NC4CC4)CC3)ccc(C(=O)Nc3cn4cc(C)nc(CNS(C)(=O)=O)c4n3)n2n1] |
| 807 | 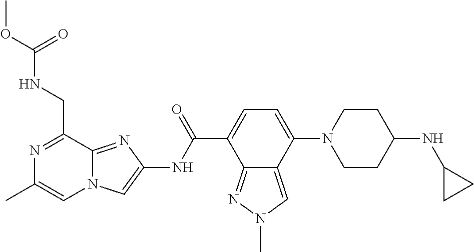 [SMILES: COC(=O)NCc1nc(C)cn2cc(NC(=O)c3ccc(N4CCC(NC5CC5)CC4)c4cn(C)nc34)nc12] |
| 808 | 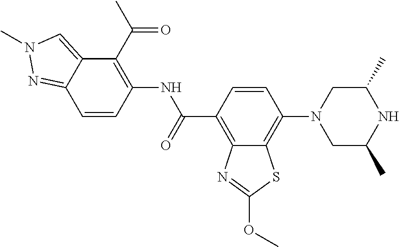 |
| 809 | 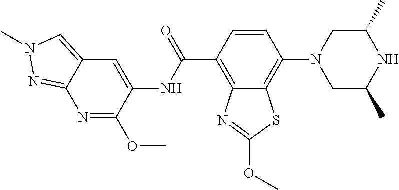 |
| 810 | 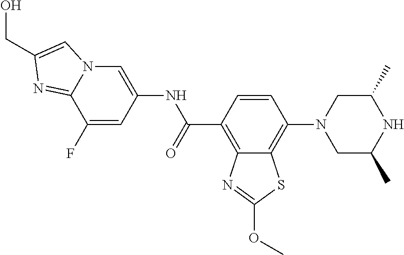 [SMILES: COc1nc2c(C(=O)Nc3cc(F)c4nc(CO)cn4c3)ccc(N3C[C@H](C)N[C@@H](C)C3)c2s1] |
| 811 | 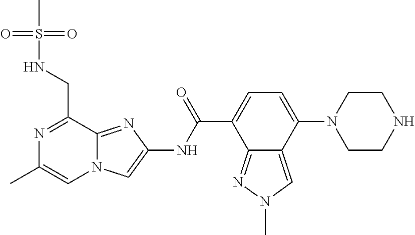 [SMILES: Cc1cn2cc(NC(=O)c3ccc(N4CCNCC4)c4cn(C)nc34)nc2c(CNS(C)(=O)=O)n1] |
| 812 | 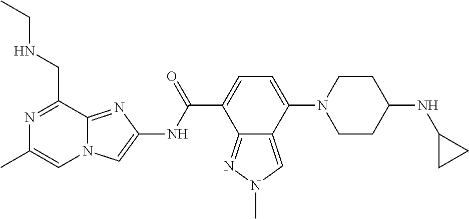 [SMILES: CCNCc1nc(C)cn2cc(NC(=O)c3ccc(N4CCC(NC5CC5)CC4)c4cn(C)nc34)nc12] |
| 813 | 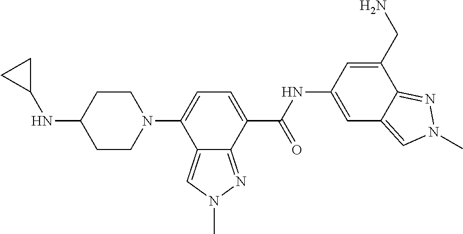 [SMILES: Cn1cc2cc(NC(=O)c3ccc(N4CCC(NC5CC5)CC4)c4cn(C)nc34)cc(CN)c2n1] |
| 814 | 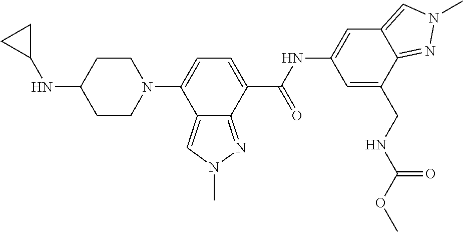 [SMILES: COC(=O)NCc1cc(NC(=O)c2ccc(N3CCC(NC4CC4)CC3)c3cn(C)nc23)cc2cn(C)nc12] |
| 815 | 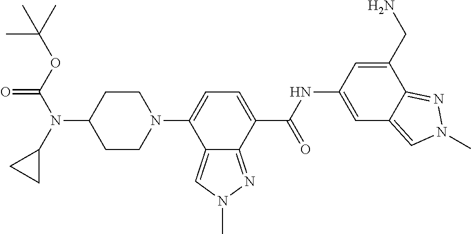 [SMILES: Cn1cc2cc(NC(=O)c3ccc(N4CCC(N(C(=O)OC(C)(C)C)C5CC5)CC4)c4cn(C)nc34)cc(CN)c2n1] |
| 816 | 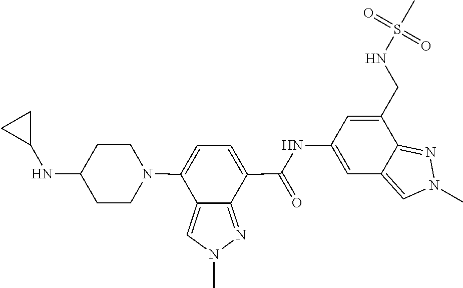 [SMILES: Cn1cc2cc(NC(=O)c3ccc(N4CCC(NC5CC5)CC4)c4cn(C)nc34)cc(CNS(C)(=O)=O)c2n1] |
| 817 | 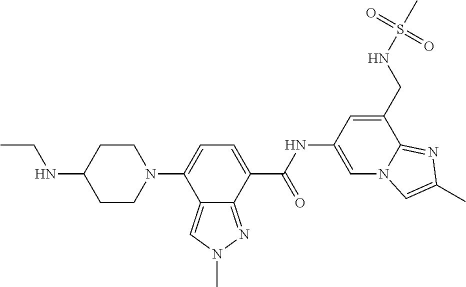 [SMILES: CCNC1CCN(c2ccc(C(=O)Nc3cc(CNS(C)(=O)=O)c4nc(C)cn4c3)c3nn(C)cc23)CC1] |
| 818 | 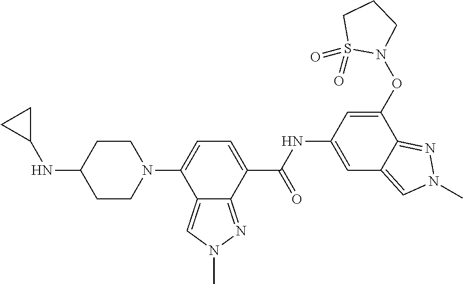 [SMILES: Cn1cc2cc(NC(=O)c3ccc(N4CCC(NC5CC5)CC4)c4cn(C)nc34)cc(ON3CCCS3(=O)=O)c2n1] |
| 819 | 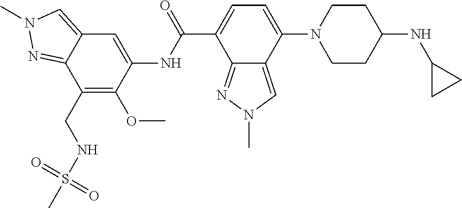 [SMILES: COc1c(NC(=O)c2ccc(N3CCC(NC4CC4)CC3)c3cn(C)nc23)cc2cn(C)nc2c1CNS(C)(=O)=O] |
| 820 | 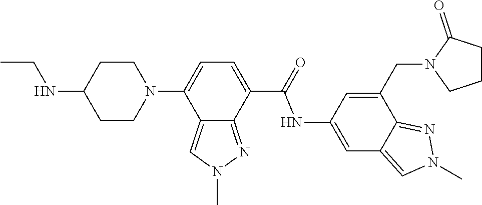 [SMILES: CCNC1CCN(c2ccc(C(=O)Nc3cc(CN4CCCC4=O)c4nn(C)cc4c3)c3nn(C)cc23)CC1] |
| 821 | 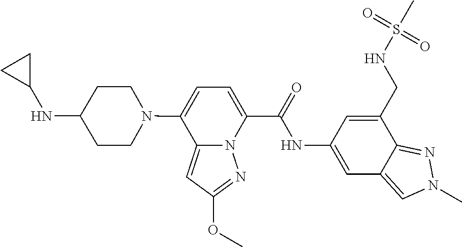 [SMILES: COc1cc2c(N3CCC(NC4CC4)CC3)ccc(C(=O)Nc3cc(CNS(C)(=O)=O)c4nn(C)cc4c3)n2n1] |
| 822 | 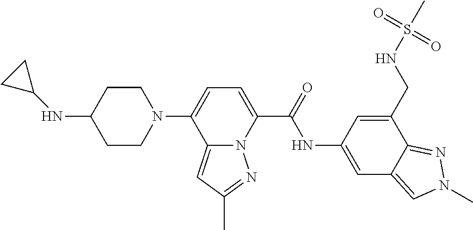 [SMILES: Cc1cc2c(N3CCC(NC4CC4)CC3)ccc(C(=O)Nc3cc(CNS(C)(=O)=O)c4nn(C)cc4c3)n2n1] |
| 823 | 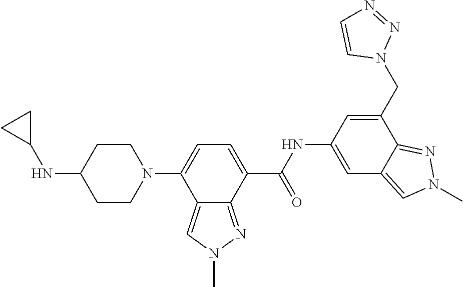 [SMILES: Cn1cc2cc(NC(=O)c3ccc(N4CCC(NC5CC5)CC4)c4cn(C)nc34)cc(Cn3ccnn3)c2n1] |
| 824 | 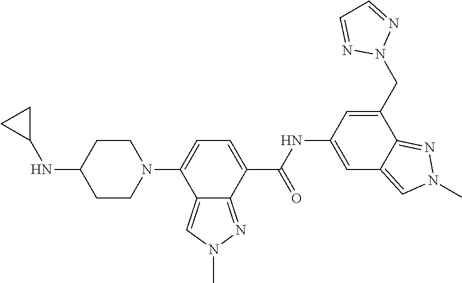 [SMILES: Cn1cc2cc(NC(=O)c3ccc(N4CCC(NC5CC5)CC4)c4cn(C)nc34)cc(Cn3nccn3)c2n1] |
| 825 | 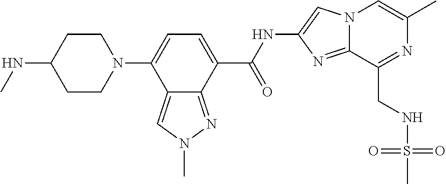 [SMILES: CNC1CCN(c2ccc(C(=O)Nc3cn4cc(C)nc(CNS(C)(=O)=O)c4n3)c3nn(C)cc23)CC1] |
| 826 | 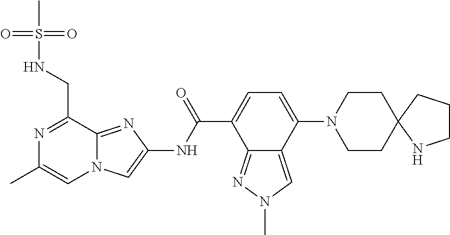 [SMILES: Cc1cn2cc(NC(=O)c3ccc(N4CCC5(CCCN5)CC4)c4cn(C)nc34)nc2c(CNS(C)(=O)=O)n1] |
| 829 | 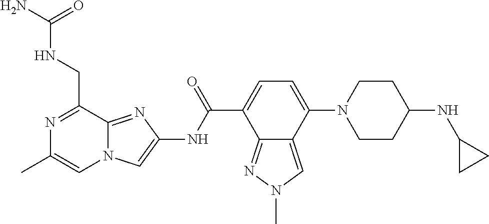 [SMILES: Cc1cn2cc(NC(=O)c3ccc(N4CCC(NC5CC5)CC4)c4cn(C)nc34)nc2c(CNC(N)=O)n1] |
| 830 | 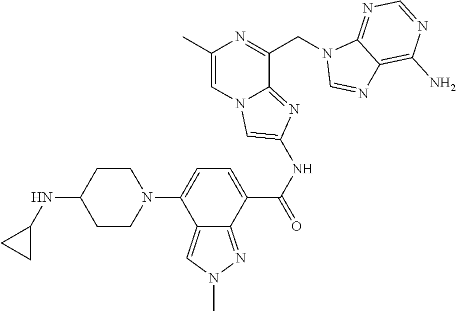 [SMILES: Cc1cn2cc(NC(=O)c3ccc(N4CCC(NC5CC5)CC4)c4cn(C)nc34)nc2c(Cn2cnc3c(N)ncnc32)n1] |
| 831 | 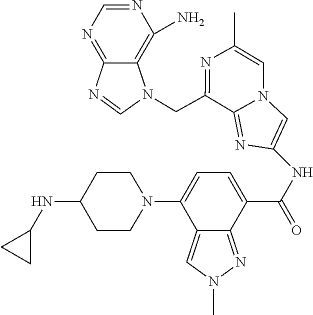 [SMILES: Cc1cn2cc(NC(=O)c3ccc(N4CCC(NC5CC5)CC4)c4cn(C)nc34)nc2c(Cn2cnc3ncnc(N)c32)n1] |
| 832 | 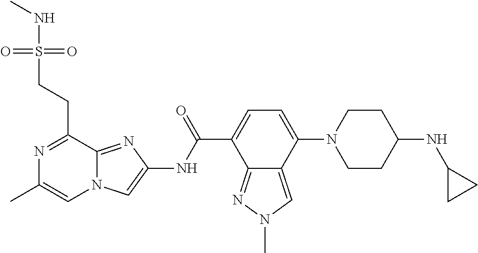 [SMILES: CNS(=O)(=O)CCc1nc(C)cn2cc(NC(=O)c3ccc(N4CCC(NC5CC5)CC4)c4cn(C)nc34)nc12] |
| 833 | 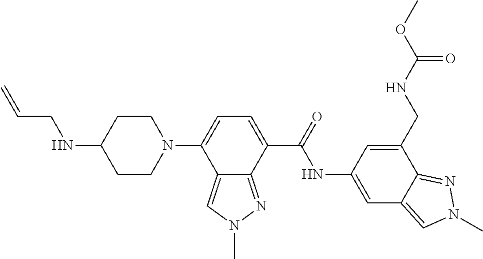 [SMILES: C=CCNC1CCN(c2ccc(C(=O)Nc3cc(CNC(=O)OC)c4nn(C)cc4c3)c3nn(C)cc23)CC1] |
| 834 | 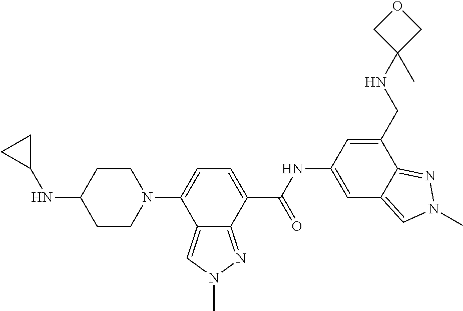 [SMILES: Cn1cc2cc(NC(=O)c3ccc(N4CCC(NC5CC5)CC4)c4cn(C)nc34)cc(CNC3(C)COC3)c2n1] |
| 835 | 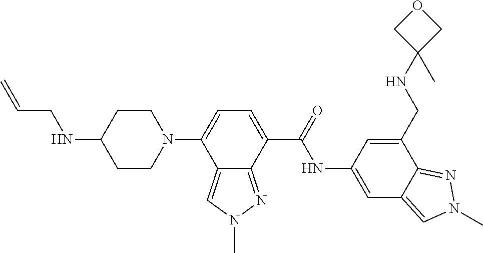 [SMILES: C=CCNC1CCN(c2ccc(C(=O)Nc3cc(CNC4(C)COC4)c4nn(C)cc4c3)c3nn(C)cc23)CC1] |
| 836 | 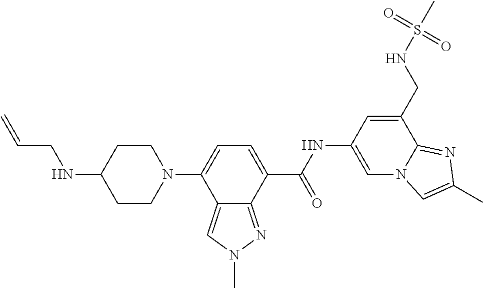 [SMILES: C=CCNC1CCN(c2ccc(C(=O)Nc3cc(CNS(C)(=O)=O)c4nc(C)cn4c3)c3nn(C)cc23)CC1] |
| 837 | 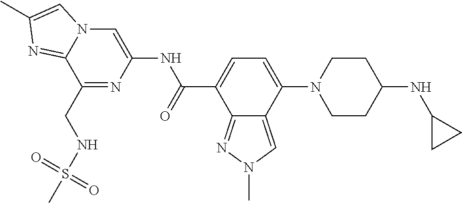 [SMILES: Cc1cn2cc(NC(=O)c3ccc(N4CCC(NC5CC5)CC4)c4cn(C)nc34)nc(CNS(C)(=O)=O)c2n1] |
| 838 | 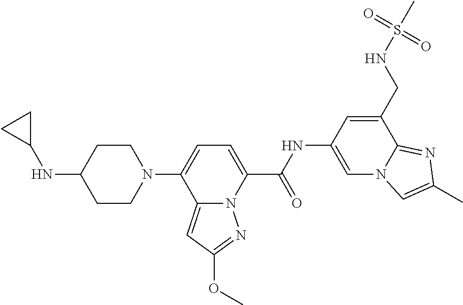 [SMILES: COc1cc2c(N3CCC(NC4CC4)CC3)ccc(C(=O)Nc3cc(CNS(C)(=O)=O)c4nc(C)cn4c3)n2n1] |
| 839 | 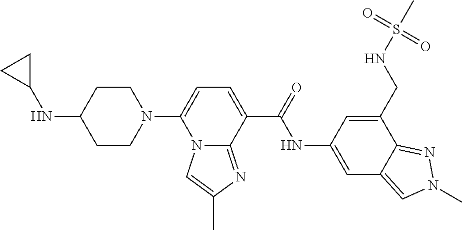 [SMILES: Cc1cn2c(N3CCC(NC4CC4)CC3)ccc(C(=O)Nc3cc(CNS(C)(=O)=O)c4nn(C)cc4c3)c2n1] |
| 840 | 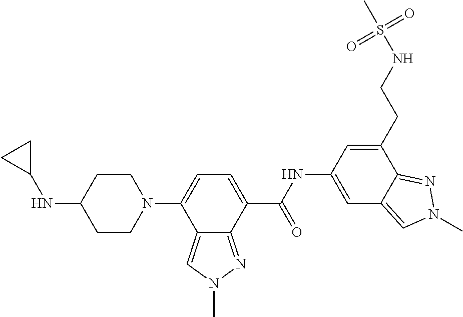 [SMILES: Cn1cc2cc(NC(=O)c3ccc(N4CCC(NC5CC5)CC4)c4cn(C)nc34)cc(CCNS(C)(=O)=O)c2n1] |
| 841 | 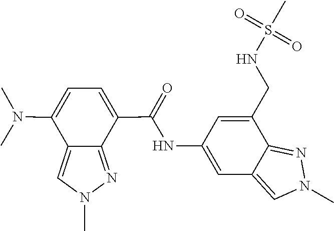 |
| 842 | 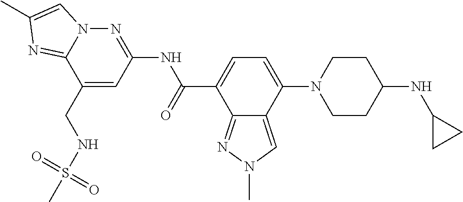 [SMILES: Cc1cn2nc(NC(=O)c3ccc(N4CCC(NC5CC5)CC4)c4cn(C)nc34)cc(CNS(C)(=O)=O)c2n1] |
| 843 | 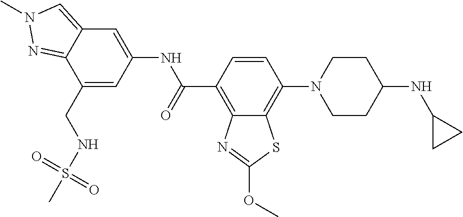 [SMILES: COc1nc2c(C(=O)Nc3cc(CNS(C)(=O)=O)c4nn(C)cc4c3)ccc(N3CCC(NC4CC4)CC3)c2s1] |
| 844 | 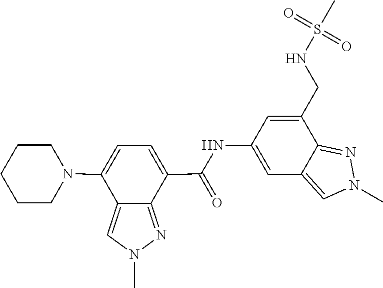 [SMILES: Cn1cc2cc(NC(=O)c3ccc(N4CCCCC4)c4cn(C)nc34)cc(CNS(C)(=O)=O)c2n1] |
| 845 | 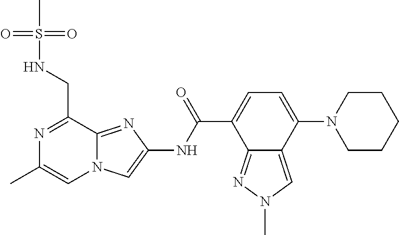 [SMILES: Cc1cn2cc(NC(=O)c3ccc(N4CCCCC4)c4cn(C)nc34)nc2c(CNS(C)(=O)=O)n1] |
| 846 | 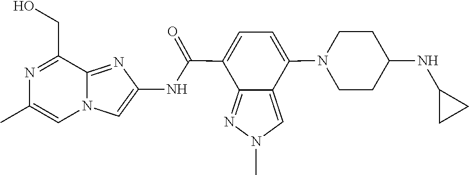 [SMILES: Cc1cn2cc(NC(=O)c3ccc(N4CCC(NC5CC5)CC4)c4cn(C)nc34)nc2c(CO)n1] |
| 847 | 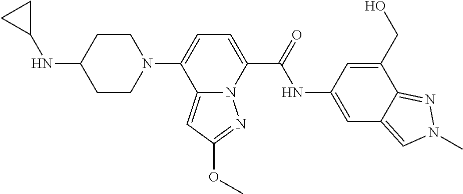 [SMILES: COc1cc2c(N3CCC(NC4CC4)CC3)ccc(C(=O)Nc3cc(CO)c4nn(C)cc4c3)n2n1] |
| 848 | 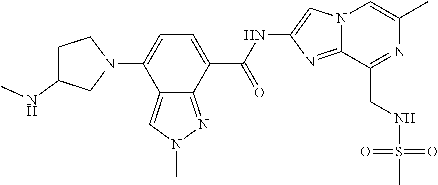 [SMILES: CNC1CCN(c2ccc(C(=O)Nc3cn4cc(C)nc(CNS(C)(=O)=O)c4n3)c3nn(C)cc23)C1] |
| 849 | 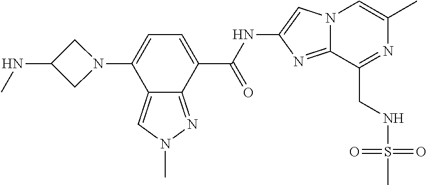 [SMILES: CNC1CN(c2ccc(C(=O)Nc3cn4cc(C)nc(CNS(C)(=O)=O)c4n3)c3nn(C)cc23)C1] |
| 850 | 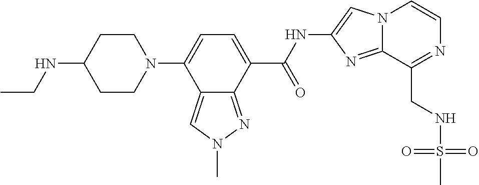 [SMILES: CCNC1CCN(c2ccc(C(=O)Nc3cn4ccnc(CNS(C)(=O)=O)c4n3)c3nn(C)cc23)CC1] |
| 851 | 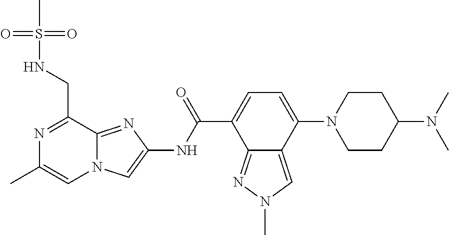 [SMILES: Cc1cn2cc(NC(=O)c3ccc(N4CCC(N(C)C)CC4)c4cn(C)nc34)nc2c(CNS(C)(=O)=O)n1] |
| 852 | 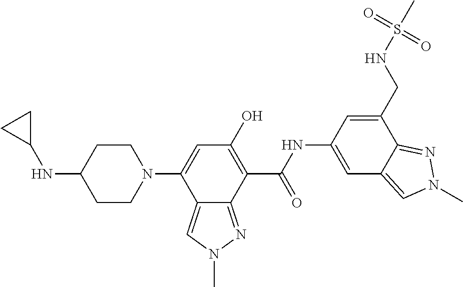 [SMILES: Cn1cc2cc(NC(=O)c3c(O)cc(N4CCC(NC5CC5)CC4)c4cn(C)nc34)cc(CNS(C)(=O)=O)c2n1] |
| 853 | 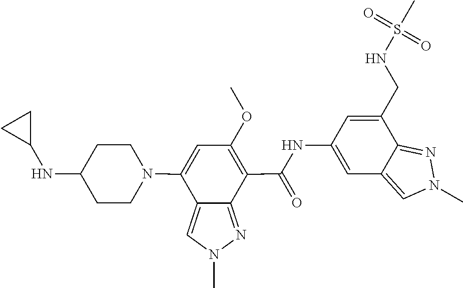 [SMILES: COc1cc(N2CCC(NC3CC3)CC2)c2cn(C)nc2c1C(=O)Nc1cc(CNS(C)(=O)=O)c2nn(C)cc2c1] |
| 854 | 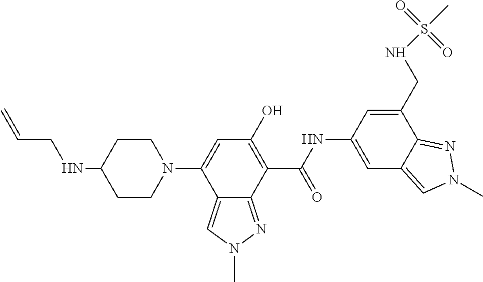 [SMILES: C=CCNC1CCN(c2cc(O)c(C(=O)Nc3cc(CNS(C)(=O)=O)c4nn(C)cc4c3)c3nn(C)cc23)CC1] |
| 855 | 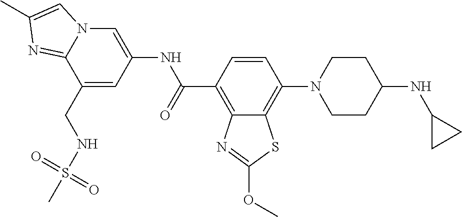 [SMILES: COc1nc2c(C(=O)Nc3cc(CNS(C)(=O)=O)c4nc(C)cn4c3)ccc(N3CCC(NC4CC4)CC3)c2s1] |
| 856 | 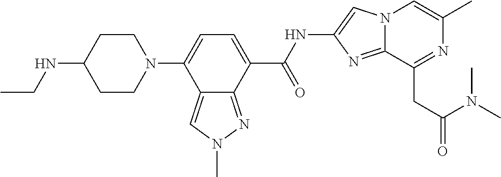 [SMILES: CCNC1CCN(c2ccc(C(=O)Nc3cn4cc(C)nc(CC(=O)N(C)C)c4n3)c3nn(C)cc23)CC1] |
| 857 | 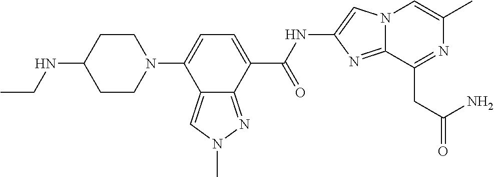 [SMILES: CCNC1CCN(c2ccc(C(=O)Nc3cn4cc(C)nc(CC(N)=O)c4n3)c3nn(C)cc23)CC1] |
| 858 | 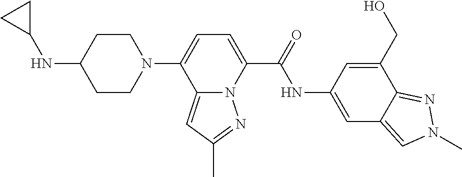 [SMILES: Cc1cc2c(N3CCC(NC4CC4)CC3)ccc(C(=O)Nc3cc(CO)c4nn(C)cc4c3)n2n1] |
| 859 | 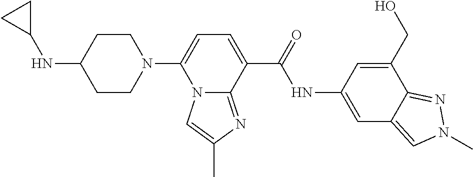 [SMILES: Cc1cn2c(N3CCC(NC4CC4)CC3)ccc(C(=O)Nc3cc(CO)c4nn(C)cc4c3)c2n1] |
| 860 | 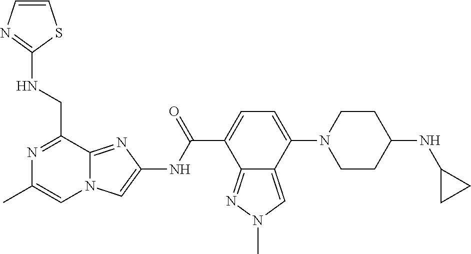 [SMILES: Cc1cn2cc(NC(=O)c3ccc(N4CCC(NC5CC5)CC4)c4cn(C)nc34)nc2c(CNc2nccs2)n1] |
| 861 | 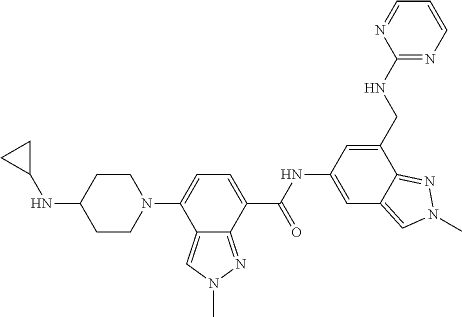 [SMILES: Cn1cc2cc(NC(=O)c3ccc(N4CCC(NC5CC5)CC4)c4cn(C)nc34)cc(CNc3ncccn3)c2n1] |
| 862 | 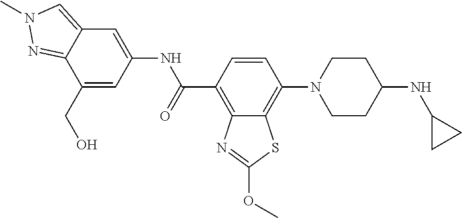 [SMILES: COc1nc2c(C(=O)Nc3cc(CO)c4nn(C)cc4c3)ccc(N3CCC(NC4CC4)CC3)c2s1] |
| 863 | 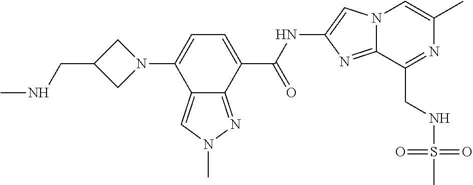 [SMILES: CNCC1CN(c2ccc(C(=O)Nc3cn4cc(C)nc(CNS(C)(=O)=O)c4n3)c3nn(C)cc23)C1] |
| 864 | 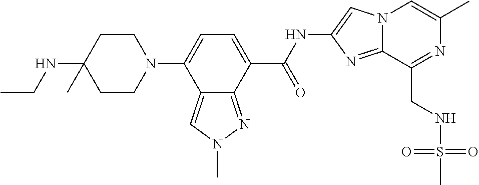 [SMILES: CCNC1(C)CCN(c2ccc(C(=O)Nc3cn4cc(C)nc(CNS(C)(=O)=O)c4n3)c3nn(C)cc23)CC1] |
| 865 | 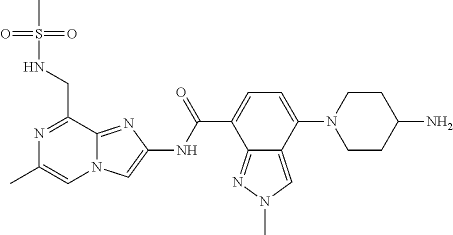 [SMILES: Cc1cn2cc(NC(=O)c3ccc(N4CCC(N)CC4)c4cn(C)nc34)nc2c(CNS(C)(=O)=O)n1] |
| 866 | 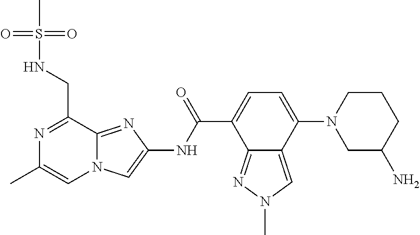 [SMILES: Cc1cn2cc(NC(=O)c3ccc(N4CCCC(N)C4)c4cn(C)nc34)nc2c(CNS(C)(=O)=O)n1] |
| 867 | 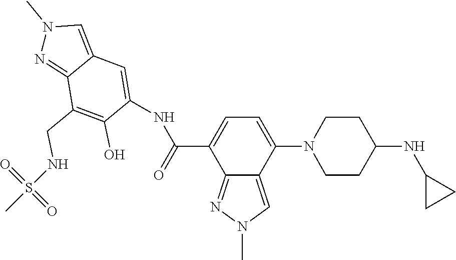 [SMILES: Cn1cc2cc(NC(=O)c3ccc(N4CCC(NC5CC5)CC4)c4cn(C)nc34)c(O)c(CNS(C)(=O)=O)c2n1] |
| 868 | 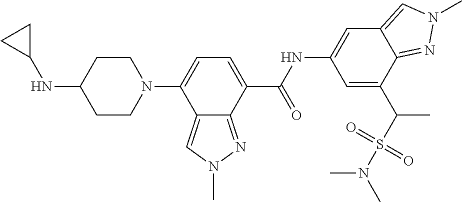 [SMILES: CC(c1cc(NC(=O)c2ccc(N3CCC(NC4CC4)CC3)c3cn(C)nc23)cc2cn(C)nc12)S(=O)(=O)N(C)C] |
| 869 | 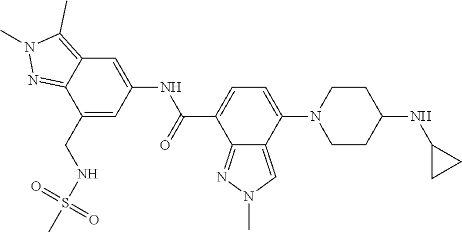 [SMILES: Cc1c2cc(NC(=O)c3ccc(N4CCC(NC5CC5)CC4)c4cn(C)nc34)cc(CNS(C)(=O)=O)c2nn1C] |
| 870 | 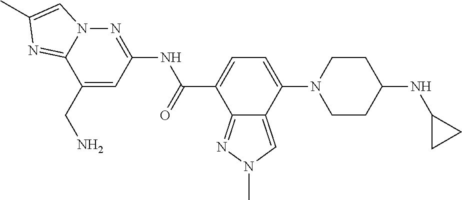 [SMILES: Cc1cn2nc(NC(=O)c3ccc(N4CCC(NC5CC5)CC4)c4cn(C)nc34)cc(CN)c2n1] |
| 871 | 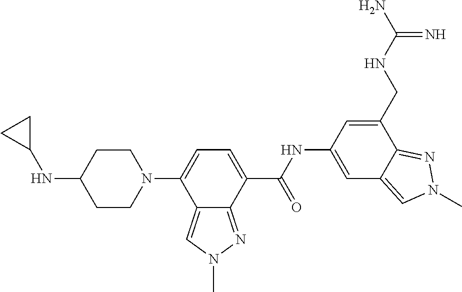 [SMILES: Cn1cc2cc(NC(=O)c3ccc(N4CCC(NC5CC5)CC4)c4cn(C)nc34)cc(CNC(=N)N)c2n1] |
| 872 | 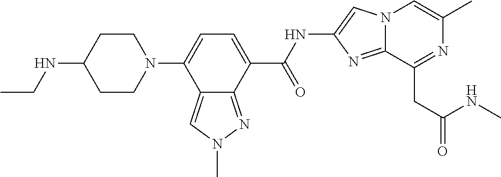 [SMILES: CCNC1CCN(c2ccc(C(=O)Nc3cn4cc(C)nc(CC(=O)NC)c4n3)c3nn(C)cc23)CC1] |
| 873 | 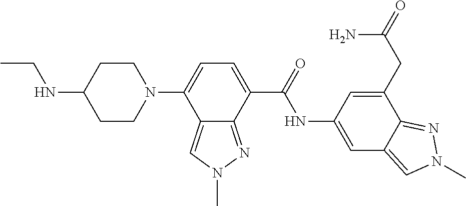 [SMILES: CCNC1CCN(c2ccc(C(=O)Nc3cc(CC(N)=O)c4nn(C)cc4c3)c3nn(C)cc23)CC1] |
| 874 | 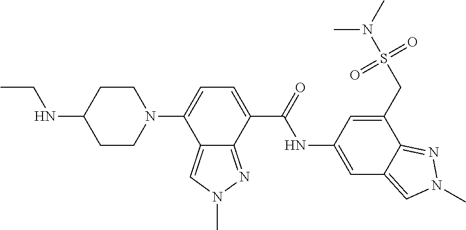 [SMILES: CCNC1CCN(c2ccc(C(=O)Nc3cc(CS(=O)(=O)N(C)C)c4nn(C)cc4c3)c3nn(C)cc23)CC1] |
| 875 | 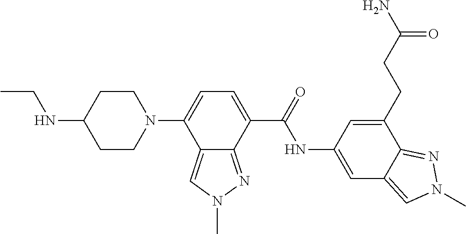 [SMILES: CCNC1CCN(c2ccc(C(=O)Nc3cc(CCC(N)=O)c4nn(C)cc4c3)c3nn(C)cc23)CC1] |
| 876 | 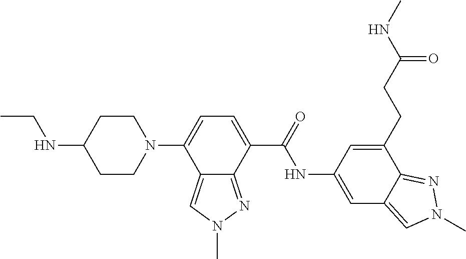 [SMILES: CCNC1CCN(c2ccc(C(=O)Nc3cc(CCC(=O)NC)c4nn(C)cc4c3)c3nn(C)cc23)CC1] |
| 877 | 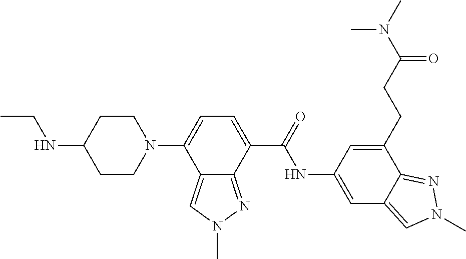 [SMILES: CCNC1CCN(c2ccc(C(=O)Nc3cc(CCC(=O)N(C)C)c4nn(C)cc4c3)c3nn(C)cc23)CC1] |
| 878 | 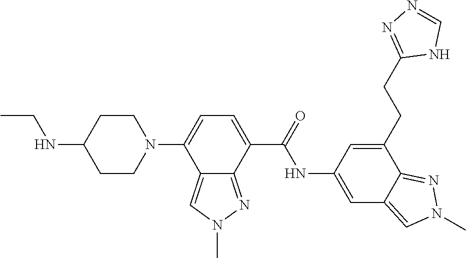 [SMILES: CCNC1CCN(c2ccc(C(=O)Nc3cc(CCc4nnc[nH]4)c4nn(C)cc4c3)c3nn(C)cc23)CC1] |
| 879 | 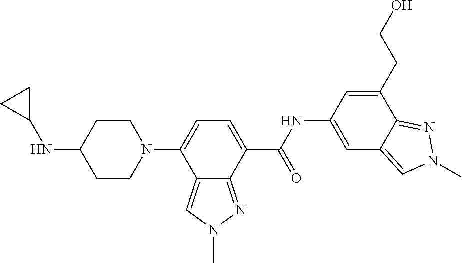 [SMILES: Cn1cc2cc(NC(=O)c3ccc(N4CCC(NC5CC5)CC4)c4cn(C)nc34)cc(CCO)c2n1] |
| 880 | 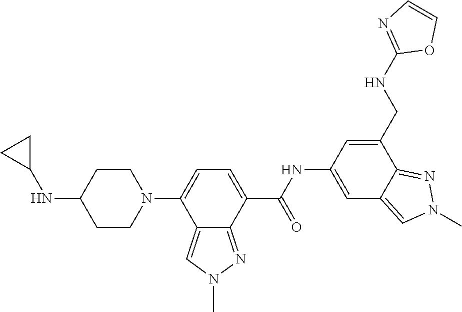 [SMILES: Cn1cc2cc(NC(=O)c3ccc(N4CCC(NC5CC5)CC4)c4cn(C)nc34)cc(CNc3ncco3)c2n1] |
| 881 | 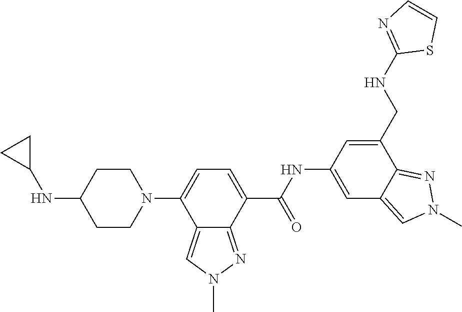 [SMILES: Cn1cc2cc(NC(=O)c3ccc(N4CCC(NC5CC5)CC4)c4cn(C)nc34)cc(CNc3nccs3)c2n1] |
| 882 | 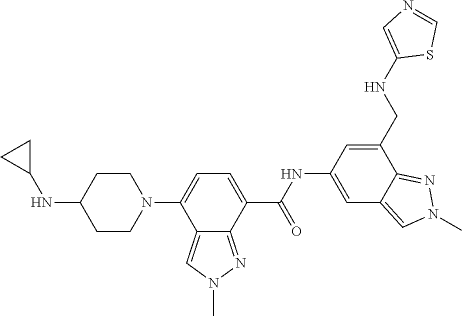 [SMILES: Cn1cc2cc(NC(=O)c3ccc(N4CCC(NC5CC5)CC4)c4cn(C)nc34)cc(CNc3cncs3)c2n1] |
| 883 | 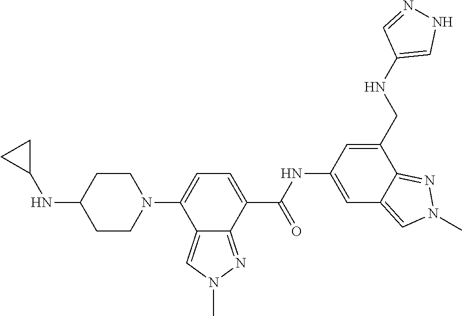 [SMILES: Cn1cc2cc(NC(=O)c3ccc(N4CCC(NC5CC5)CC4)c4cn(C)nc34)cc(CNc3cn[nH]c3)c2n1] |
| 884 | 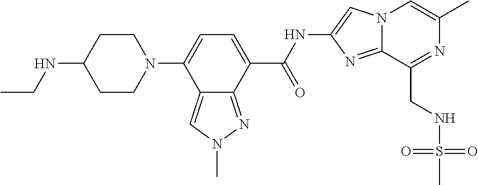 [SMILES: CCNC1CCN(c2ccc(C(=O)Nc3cn4cc(C)nc(CNS(C)(=O)=O)c4n3)c3nn(C)cc23)CC1] |
| 885 | 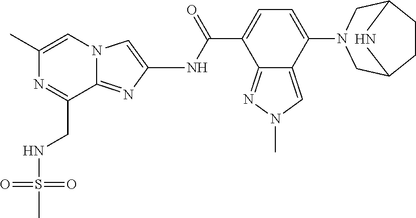 [SMILES: Cc1cn2cc(NC(=O)c3ccc(N4CC5CCC(C4)N5)c4cn(C)nc34)nc2c(CNS(C)(=O)=O)n1] |
| 886 | 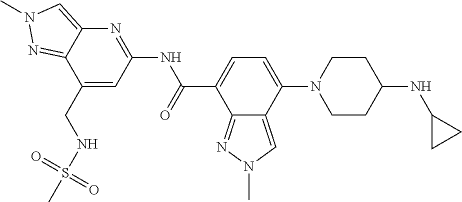 [SMILES: Cn1cc2nc(NC(=O)c3ccc(N4CCC(NC5CC5)CC4)c4cn(C)nc34)cc(CNS(C)(=O)=O)c2n1] |
| 887 | 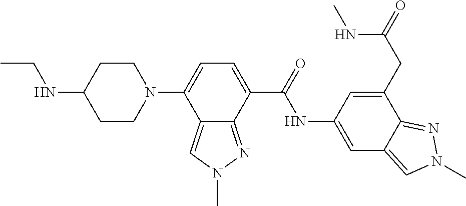 [SMILES: CCNC1CCN(c2ccc(C(=O)Nc3cc(CC(=O)NC)c4nn(C)cc4c3)c3nn(C)cc23)CC1] |
| 888 | 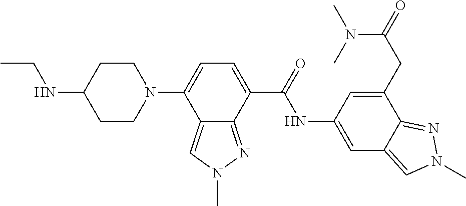 [SMILES: CCNC1CCN(c2ccc(C(=O)Nc3cc(CC(=O)N(C)C)c4nn(C)cc4c3)c3nn(C)cc23)CC1] |
| 889 | 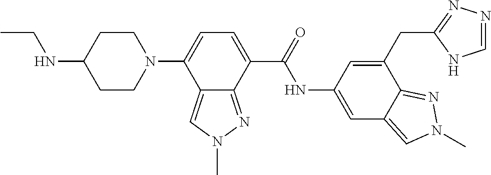 [SMILES: CCNC1CCN(c2ccc(C(=O)Nc3cc(Cc4nnc[nH]4)c4nn(C)cc4c3)c3nn(C)cc23)CC1] |
| 890 | 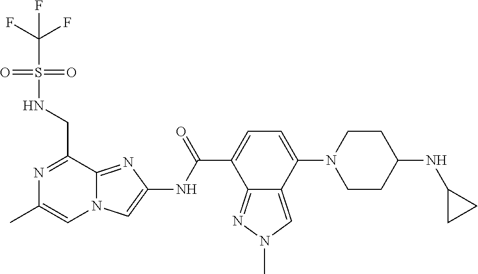 [SMILES: Cc1cn2cc(NC(=O)c3ccc(N4CCC(NC5CC5)CC4)c4cn(C)nc34)nc2c(CNS(=O)(=O)C(F)(F)F)n1] |
| 891 | 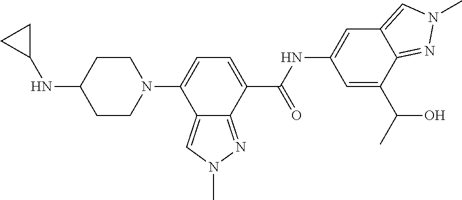 [SMILES: CC(O)c1cc(NC(=O)c2ccc(N3CCC(NC4CC4)CC3)c3cn(C)nc23)cc2cn(C)nc12] |
| 892 | 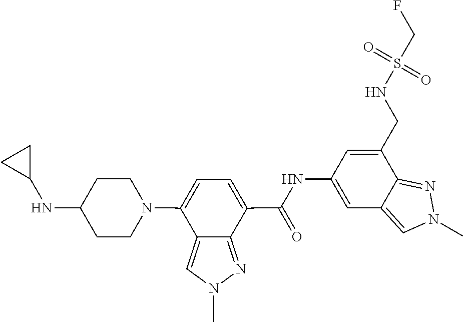 [SMILES: Cn1cc2cc(NC(=O)c3ccc(N4CCC(NC5CC5)CC4)c4cn(C)nc34)cc(CNS(=O)(=O)CF)c2n1] |
| 893 | 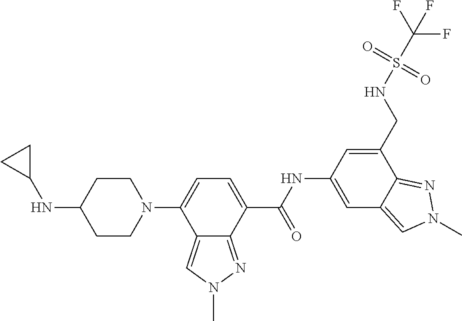 [SMILES: Cn1cc2cc(NC(=O)c3ccc(N4CCC(NC5CC5)CC4)c4cn(C)nc34)cc(CNS(=O)(=O)C(F)(F)F)c2n1] |
| 894 | 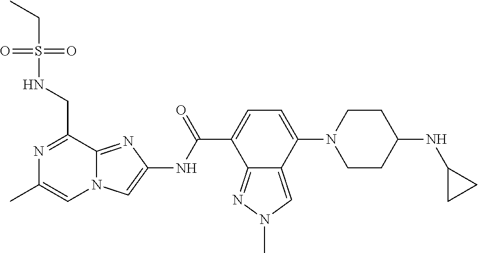 [SMILES: CCS(=O)(=O)NCc1nc(C)cn2cc(NC(=O)c3ccc(N4CCC(NC5CC5)CC4)c4cn(C)nc34)nc12] |
| 895 | 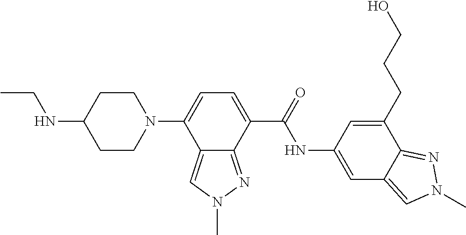 [SMILES: CCNC1CCN(c2ccc(C(=O)Nc3cc(CCCO)c4nn(C)cc4c3)c3nn(C)cc23)CC1] |
| 896 | 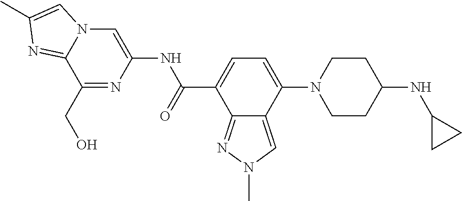 [SMILES: Cc1cn2cc(NC(=O)c3ccc(N4CCC(NC5CC5)CC4)c4cn(C)nc34)nc(CO)c2n1] |
| 897 | 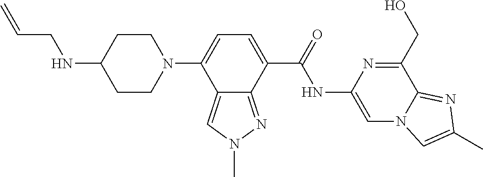 [SMILES: C=CCNC1CCN(c2ccc(C(=O)Nc3cn4cc(C)nc4c(CO)n3)c3nn(C)cc23)CC1] |
| 898 | 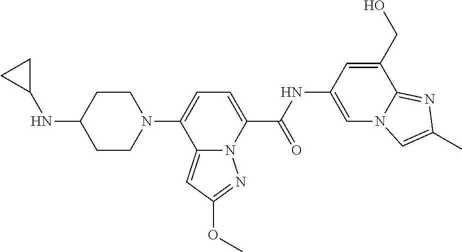 [SMILES: COc1cc2c(N3CCC(NC4CC4)CC3)ccc(C(=O)Nc3cc(CO)c4nc(C)cn4c3)n2n1] |
| 899 | 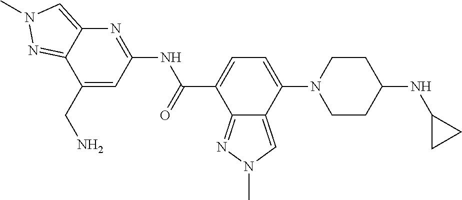 [SMILES: Cn1cc2nc(NC(=O)c3ccc(N4CCC(NC5CC5)CC4)c4cn(C)nc34)cc(CN)c2n1] |
| 900 | 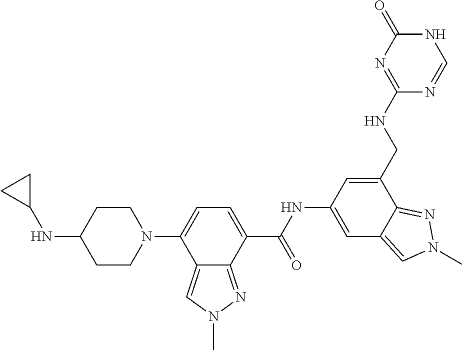 [SMILES: Cn1cc2cc(NC(=O)c3ccc(N4CCC(NC5CC5)CC4)c4cn(C)nc34)cc(CNc3nc[nH]c(=O)n3)c2n1] |
| 901 | 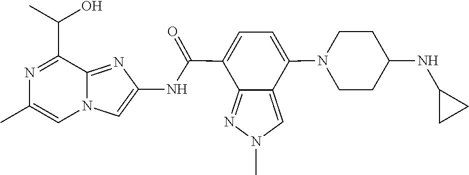 [SMILES: Cc1cn2cc(NC(=O)c3ccc(N4CCC(NC5CC5)CC4)c4cn(C)nc34)nc2c(C(C)O)n1] |
| 902 | 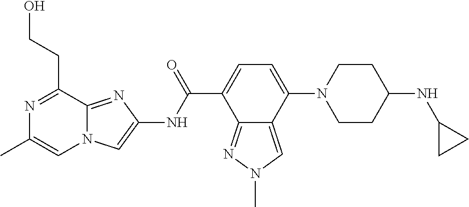 [SMILES: Cc1cn2cc(NC(=O)c3ccc(N4CCC(NC5CC5)CC4)c4cn(C)nc34)nc2c(CCO)n1] |
| 903 | 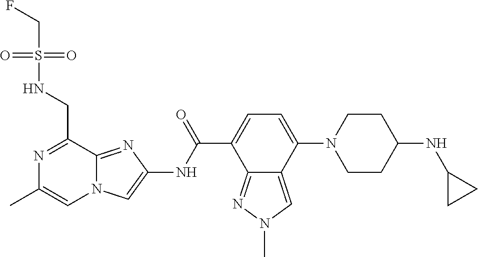 [SMILES: Cc1cn2cc(NC(=O)c3ccc(N4CCC(NC5CC5)CC4)c4cn(C)nc34)nc2c(CNS(=O)(=O)CF)n1] |
| 904 | 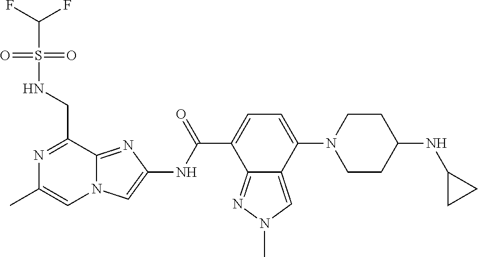 [SMILES: Cc1cn2cc(NC(=O)c3ccc(N4CCC(NC5CC5)CC4)c4cn(C)nc34)nc2c(CNS(=O)(=O)C(F)F)n1] |
| 905 | 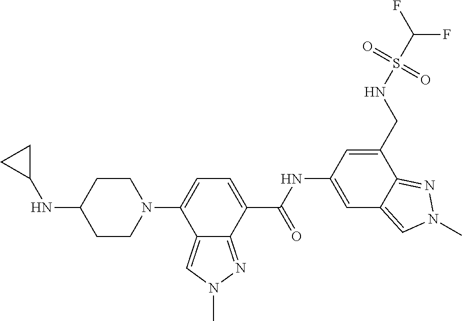 [SMILES: Cn1cc2cc(NC(=O)c3ccc(N4CCC(NC5CC5)CC4)c4cn(C)nc34)cc(CNS(=O)(=O)C(F)F)c2n1] |
| 906 | 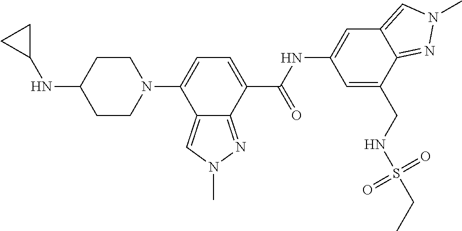 [SMILES: CCS(=O)(=O)NCc1cc(NC(=O)c2ccc(N3CCC(NC4CC4)CC3)c3cn(C)nc23)cc2cn(C)nc12] |
| 907 | 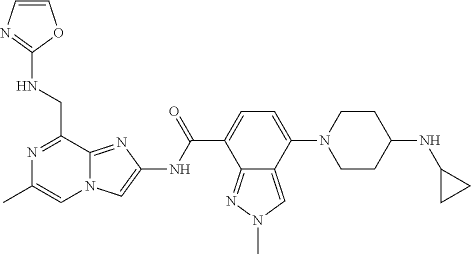 [SMILES: Cc1cn2cc(NC(=O)c3ccc(N4CCC(NC5CC5)CC4)c4cn(C)nc34)nc2c(CNc2ncco2)n1] |
| 908 | 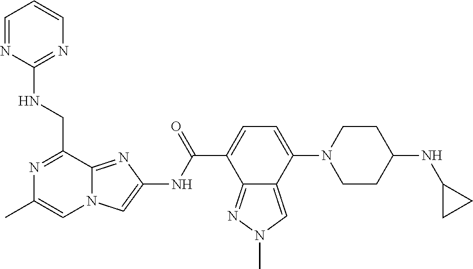 [SMILES: Cc1cn2cc(NC(=O)c3ccc(N4CCC(NC5CC5)CC4)c4cn(C)nc34)nc2c(CNc2ncccn2)n1] |
| 909 | 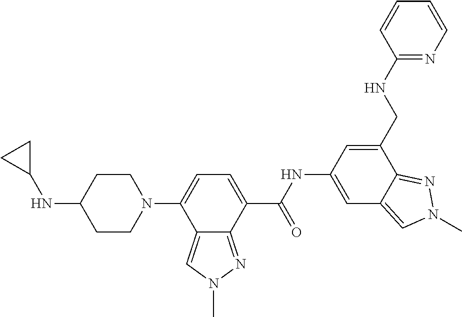 [SMILES: Cn1cc2cc(NC(=O)c3ccc(N4CCC(NC5CC5)CC4)c4cn(C)nc34)cc(CNc3ccccn3)c2n1] |
| 910 | 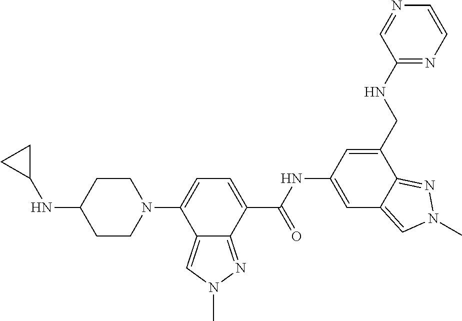 [SMILES: Cn1cc2cc(NC(=O)c3ccc(N4CCC(NC5CC5)CC4)c4cn(C)nc34)cc(CNc3cnccn3)c2n1] |
| 911 | 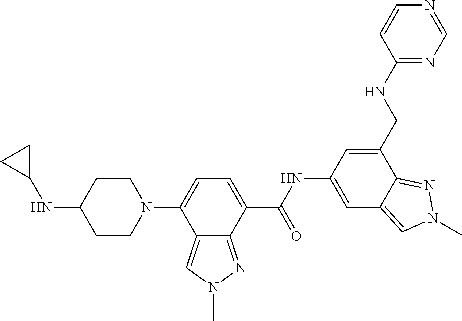 [SMILES: Cn1cc2cc(NC(=O)c3ccc(N4CCC(NC5CC5)CC4)c4cn(C)nc34)cc(CNc3ccncn3)c2n1] |
| 912 | 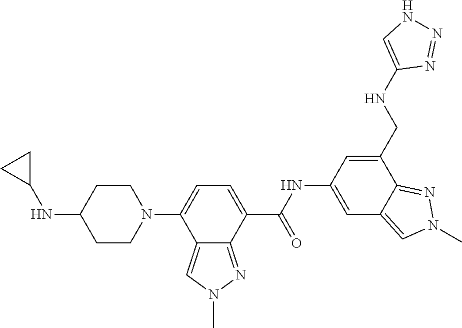 [SMILES: Cn1cc2cc(NC(=O)c3ccc(N4CCC(NC5CC5)CC4)c4cn(C)nc34)cc(CNc3c[nH]nn3)c2n1] |
| 913 | 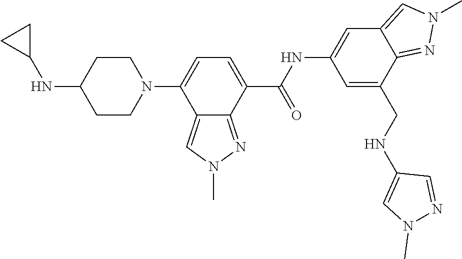 [SMILES: Cn1cc(NCc2cc(NC(=O)c3ccc(N4CCC(NC5CC5)CC4)c4cn(C)nc34)cc3cn(C)nc23)cn1] |
| 914 | 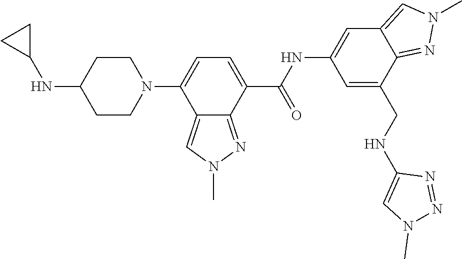 [SMILES: Cn1cc(NCc2cc(NC(=O)c3ccc(N4CCC(NC5CC5)CC4)c4cn(C)nc34)cc3cn(C)nc23)nn1] |
| 915 | 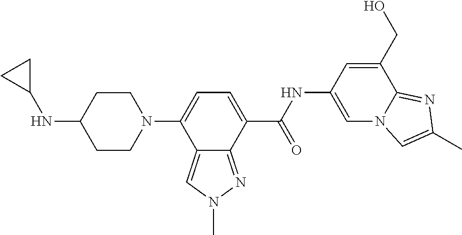 [SMILES: Cc1cn2cc(NC(=O)c3ccc(N4CCC(NC5CC5)CC4)c4cn(C)nc34)cc(CO)c2n1] |
| 916 | 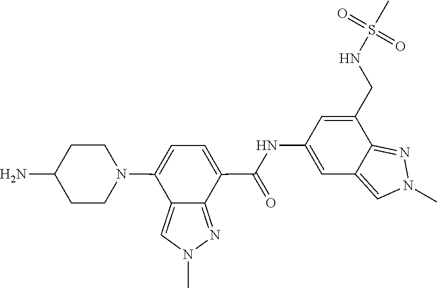 [SMILES: Cn1cc2cc(NC(=O)c3ccc(N4CCC(N)CC4)c4cn(C)nc34)cc(CNS(C)(=O)=O)c2n1] |
| 917 | 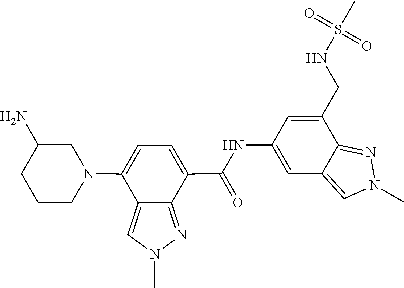 [SMILES: Cn1cc2cc(NC(=O)c3ccc(N4CCCC(N)C4)c4cn(C)nc34)cc(CNS(C)(=O)=O)c2n1] |
| 918 | 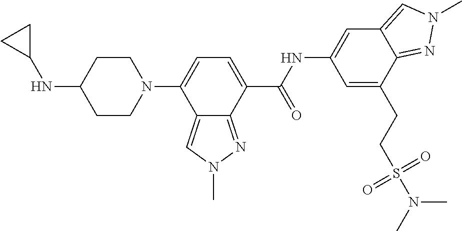 [SMILES: CN(C)S(=O)(=O)CCc1cc(NC(=O)c2ccc(N3CCC(NC4CC4)CC3)c3cn(C)nc23)cc2cn(C)nc12] |
| 919 | 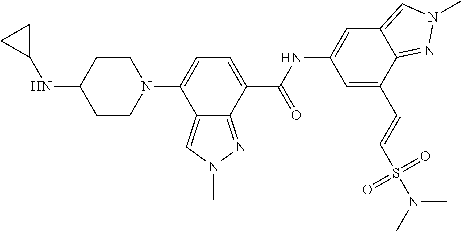 [SMILES: CN(C)S(=O)(=O)/C=C/c1cc(NC(=O)c2ccc(N3CCC(NC4CC4)CC3)c3cn(C)nc23)cc2cn(C)nc12] |
| 920 | 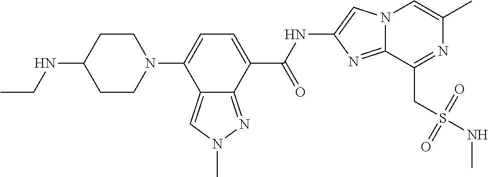 [SMILES: CCNC1CCN(c2ccc(C(=O)Nc3cn4cc(C)nc(CS(=O)(=O)NC)c4n3)c3nn(C)cc23)CC1] |
| 921 | 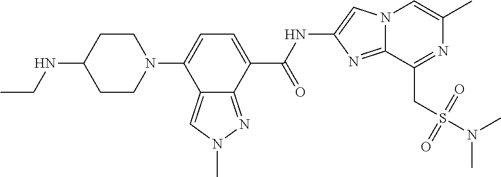 [SMILES: CCNC1CCN(c2ccc(C(=O)Nc3cn4cc(C)nc(CS(=O)(=O)N(C)C)c4n3)c3nn(C)cc23)CC1] |
| 922 | 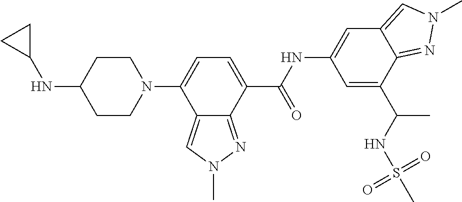 [SMILES: CC(NS(C)(=O)=O)c1cc(NC(=O)c2ccc(N3CCC(NC4CC4)CC3)c3cn(C)nc23)cc2cn(C)nc12] |
| 923 | 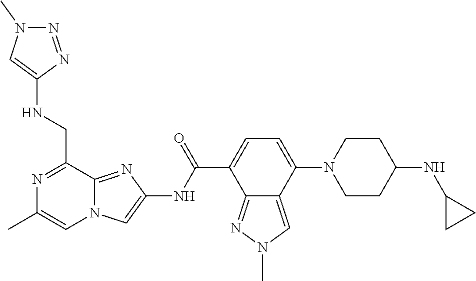 [SMILES: Cc1cn2cc(NC(=O)c3ccc(N4CCC(NC5CC5)CC4)c4cn(C)nc34)nc2c(CNc2cn(C)nn2)n1] |
| 924 | 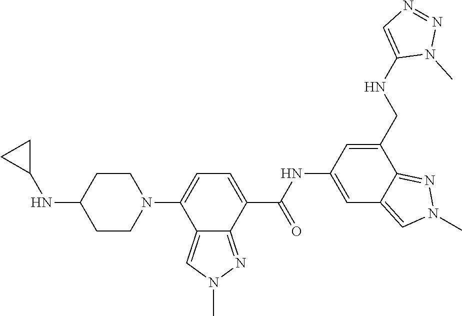 [SMILES: Cn1cc2cc(NC(=O)c3ccc(N4CCC(NC5CC5)CC4)c4cn(C)nc34)cc(CNc3cnnn3C)c2n1] |
| 925 | 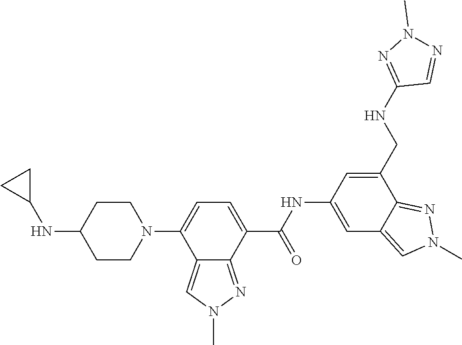 [SMILES: Cn1cc2cc(NC(=O)c3ccc(N4CCC(NC5CC5)CC4)c4cn(C)nc34)cc(CNc3cnn(C)n3)c2n1] |
| 926 | 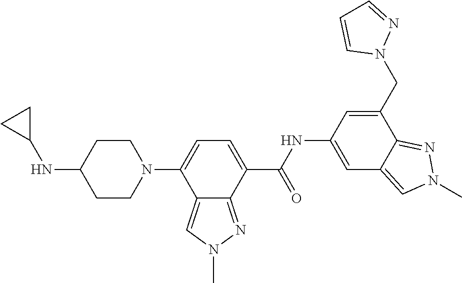 [SMILES: Cn1cc2cc(NC(=O)c3ccc(N4CCC(NC5CC5)CC4)c4cn(C)nc34)cc(Cn3cccn3)c2n1] |
| 927 | 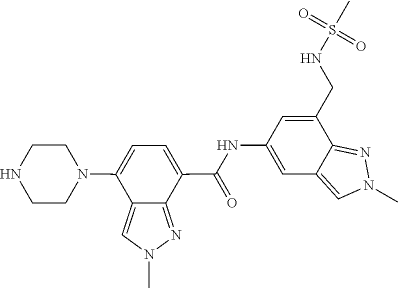 [SMILES: Cn1cc2cc(NC(=O)c3ccc(N4CCNCC4)c4cn(C)nc34)cc(CNS(C)(=O)=O)c2n1] |
| 928 | 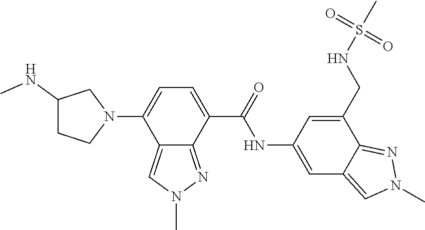 |
| 929 | 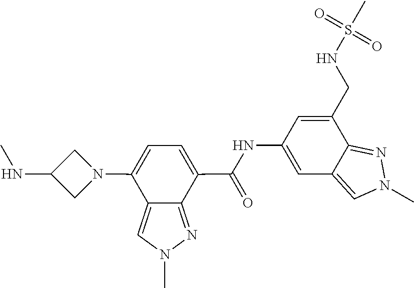 |
| 930 | 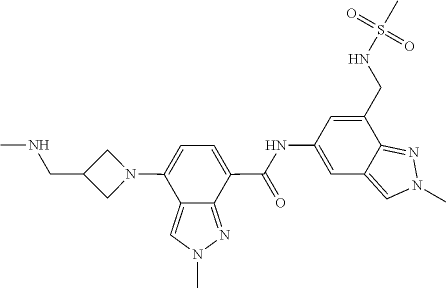 |
| 931 | 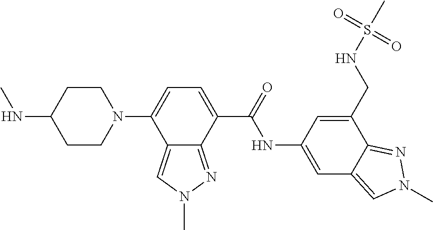 |
| 932 | 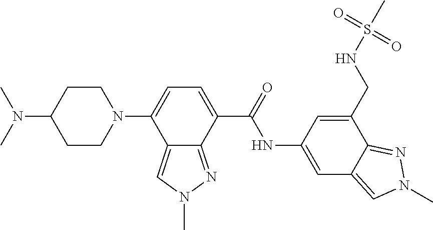 |

### Pharmaceutical Compositions, Kits, and Administration

The present invention provides pharmaceutical compositions comprising a compound of Formula (I) or (II), e.g., a compound of Formula (I) or (II) or a pharmaceutically acceptable salt, solvate, hydrate, tautomer, or stereoisomer, as described herein, and optionally a pharmaceutically acceptable excipient. In certain embodiments, the pharmaceutical composition described herein comprises a compound of Formula (I) or (II) or a pharmaceutically acceptable salt thereof, and optionally a pharmaceutically acceptable excipient. In certain embodiments, the compound of Formula (I) or (II) or a pharmaceutically acceptable salt, solvate, hydrate, tautomer, or stereoisomer thereof, is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the compound of Formula (I) or (II) (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally, or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A provided compound can also be in micro-encapsulated form.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

For ophthalmic use, provided pharmaceutically acceptable compositions may be formulated as micronized suspensions or in an ointment such as petrolatum.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of Formula (I) or (II) may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the invention are kits (e.g., pharmaceutical packs). The inventive kits may be useful for preventing and/or treating a proliferative disease or a non-proliferative disease, e.g., as described herein. The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one-unit dosage form.

Thus, in one aspect, provided are kits including a first container comprising a compound described herein, or a pharmaceutically acceptable salt, solvate, hydrate, tautomer, or stereoisomer thereof, or a pharmaceutical composition thereof. In certain embodiments, the kit of the disclosure includes a first container comprising a compound described herein, or a pharmaceutically acceptable salt thereof, or a pharmaceutical composition thereof. In certain embodiments, the kits are useful in preventing and/or treating a disease, disorder, or condition described herein in a subject (e.g., a proliferative disease or a non-proliferative disease). In certain embodiments, the kits further include instructions for administering the compound, or a pharmaceutically acceptable salt, solvate, hydrate, tautomer, or stereoisomer thereof, or a pharmaceutical composition thereof, to a subject to prevent and/or treat a proliferative disease or a non-proliferative disease.

### Methods of Use

Described herein are compounds useful for modulating splicing. In some embodiments, a compound of Formula (I) or (II) may be used to alter the amount, structure, or composition of a nucleic acid (e.g., a precursor RNA, e.g., a pre-mRNA, or the resulting mRNA) by increasing or decreasing splicing at a splice site. In some embodiments, increasing or decreasing splicing results in modulating the level or structure of a gene product (e.g., an RNA or protein) produced. In some embodiments, a compound of Formula (I) or (II) may modulate a component of the splicing machinery, e.g., by modulating the interaction with a component of the splicing machinery with another entity (e.g., nucleic acid, protein, or a combination thereof). The splicing machinery as referred to herein comprises one or more spliceosome components. Spliceosome components may comprise, for example, one or more of major spliceosome members (U1, U2, U4, U5, U6 snRNPs), or minor spliceosome members (U11, U12, U4atac, U6atac snRNPs) and their accessory splicing factors.

In another aspect, the present disclosure features a method of modifying of a target (e.g., a precursor RNA, e.g., a pre-mRNA) through inclusion of a splice site in the target, wherein the method comprises providing a compound of Formula (I) or (II). In some embodiments, inclusion of a splice site in a target (e.g., a precursor RNA, e.g., a pre-mRNA, or the resulting mRNA) results in addition or deletion of one or more nucleic acids to the target (e.g., a new exon, e.g. a skipped exon). Addition or deletion of one or more nucleic acids to the target may result in an increase in the levels of a gene product (e.g., RNA, e.g., mRNA, or protein).

In another aspect, the present disclosure features a method of modifying a target (e.g., a precursor RNA, e.g., a pre-mRNA, or the resulting mRNA) through exclusion of a splice site in the target, wherein the method comprises providing a compound of Formula (I) or (II). In some embodiments, exclusion of a splice site in a target (e.g., a precursor RNA, e.g., a pre-mRNA) results in deletion or addition of one or more nucleic acids from the target (e.g., a skipped exon, e.g. a new exon). Deletion or addition of one or more nucleic acids from the target may result in a decrease in the levels of a gene product (e.g., RNA, e.g., mRNA, or protein). In other embodiments, the methods of modifying a target (e.g., a precursor RNA, e.g., a pre-mRNA, or the resulting mRNA) comprise suppression of splicing at a splice site or enhancement of splicing at a splice site (e.g., by more than about 0.5%, e.g., 1%, 5%, 10%, 15%, 20%, 25%, 30%, 35%, 40%, 45%, 50%, 55%, 60%, 65%, 70%, 75%, 80%, 85%, 90%, 95%, 99%, or more), e.g., as compared to a reference (e.g., the absence of a compound of Formula (I) or (II), or in a healthy or diseased cell or tissue).

The methods described herein can be used to modulate splicing, e.g., of a nucleic acid comprising a particular sequence (e.g., a target sequence). Exemplary genes encoding a target sequence (e.g., a target sequence comprising DNA or RNA, e.g., pre-mRNA) include, inter alia, ABCA4, ABCA9, ABCB1, ABCB5, ABCC9, ABCD1, ACADL, ACADM, ACADSB, ACSS2, ACTB, ACTG2, ADA, ADAL, ADAM10, ADAM15, ADAM22, ADAM32, ADAMTS12, ADAMTS13, ADAMTS20, ADAMTS6, ADAMTS9, ADAR, ADCY3, ADCY10, ADCY8, ADNP, ADRBK2, AFP, AGL, AGT, AHCTF1, AHR, AKAP10, AKAP3, AKNA, ALAS1, ALS2CL, ALB, ALDH3A2, ALG6, AMBRA1, ANK3, ANTXR2, ANXA10, ANXA11, ANGPTL3, AP2A2, AP4E1, APC, APOA1, APOB, APOC3, APOH, AR, ARID2, ARID3A, ARID3B, ARFGEF1, ARFGEF2, ARHGAP1, ARHGAP8, ARHGAP18, ARHGAP26, ARHGEF18, ARHGEF2, ARPC3, ARS2, ASH1L, ASH1L-IT1, ASNSD1, ASPM, ATAD5, ATF1, ATG4A, ATG16L2, ATM, ATN1, ATP11C, ATP6V1G3, ATP13A5, ATP7A, ATP7B, ATR, ATXN2, ATXN3, ATXN7, ATXN10, AXIN1, B2M, B4GALNT3, BBS4, BCL2, BCL2L1, BCL2-like 11 (BIM), BCL11B, BBOX1, BCS1L, BEAN1, BHLHE40, BMPR2, BMP2K, BPTF, BRAF, BRCA1, BRCA2, BRCC3, BRSK1, BRSK2, BTAF1, BTK, C2orf55, C4orf29, C6orf118, C9orf43, C9orf72, C10orf137, C11orf30, C11orf65, C11orf70, C11orf87, C12orf51, C13orf1, C13orf15, C14orf101, C14orf118, C15orf29, C15orf42, C15orf60, C16orf33, C16orf38, C16orf48, C18orf8, C19orf42, C1orf107, C1orf114, C1orf130, C1orf149, C1orf27, C1orf71, C1orf94, CIR, C20orf74, C21orf70, C3orf23, C4orf18, C5orf34, C8B, C8orf33, C9orf114, C9orf86, C9orf98, C3, CA11, CAB39, CACHD1, CACNA1A, CACNA1B, CACNA1C, CACNA2D1, CACNA1G, CACNA1H, CALCA, CALCOCO2, CAMK1D, CAMKK1, CAPN3, CAPN9, CAPSL, CARD11, CARKD, CASZ1, CAT, CBLB, CBX1, CBX3, CCDC102B, CCDC11, CCDC15, CCDC18, CCDC5, CCDC81, CCDC131, CCDC146, CD4, CD274, CD1B, CDC14A, CDC16, CDC2L5, CDC42BPB, CDCA8, CDH10, CDH11, CDH24, CDH8, CDH9, CDK5RAP2, CDK6, CDK8, CDK11B, CD33, CD46, CDH1, CDH23, CDK6, CDK11B, CDK13, CEBPZ, CEL, CELSR3, CENPA, CENPI, CENPT, CENTB2, CENTG2, CEP110, CEP170, CEP192, CETP, CFB, CFTR, CFH, CGN, CGNL1, CHAF1A, CHD9, CHIC2, CHL1, CHN1, CHM, CLEC16A, CLIC2, CLCN1, CLINT1, CLK1, CLPB, CLPTM1, CM1P, CMYA5, CNGA3, CNOT1, CNOT7, CNTN6, COG3, COL11A1, COL11A2, COL12A1, COL14A1, COL15A1, COL17A1, COL19A1, COL1A1, COL1A2, COL2A1, COL3A1, COL4A1, COL4A2, COL4A5, COL4A6, COL5A2, COL6A1, COL7A1, COL9A1, COL9A2, COL22A1, COL24A1, COL25A1, COL29A1, COLQ, COMTD1, COPA, COPB2, COPS7B, COPZ2, CPSF2, CPXM2, CR1, CRBN, CRYZ, CREBBP, CRKRS, CSE1L, CSTB, CSTF3, CT45-6, CTNNB1, CUBN, CUL4B, CUL5, CXorf41, CXXC1, CYBB, CYF1P2, CYP3A4, CYP3A43, CYP3A5, CYP4F2, CYP4F3, CYP17, CYP19, CYP24A1, CYP27A1, DAB1, DAZ2, DCBLD1, DCC, DCTN3, DCUN1D4, DDA1, DDEF1, DDX1, DDX24, DDX4, DENND2D, DEPDC2, DES, DGAT2, DHFR, DHRS7, DHRS9, DHX8, DIP2A, DMD, DMTF1, DNAH3, DNAH8, DNAI1, DNAJA4, DNAJC13, DNAJC7, DNMT1, DNTTIP2, DOCK4, DOCK5, DOCK10, DOCK11, DOT1L, DPP3, DPP4, DPY19L2P2, DR1, DSCC1, DVL3, DUX4, DYNC1H1, DYSF, E2F1, E2F3, E2F8, E4F1, EBF1, EBF3, ECM2, EDEM3, EFCAB3, EFCAB4B, EFNA4, EFTUD2, EGFR, EIF3A, ELA1, ELA2A, ELF2, ELF3, ELF4, EMCN, EMD, EML5, ENO3, ENPP3, EP300, EPAS1, EPB41L5, EPHA3, EPHA4, EPHB1, EPHB2, EPHB3, EPS15, ERBB4, ERCC1, ERCC8, ERGIC3, ERMN, ERMP1, ERN1, ERN2, ESR1, ESRRG, ETS2, ETV3, ETV4, ETV5, ETV6, EVC2, EWSR1, EXO1, EXOC4, F3, F11, F13A1, F5, F7, F8, FAH, FAM13A1, FAM13B1, FAM13C1, FAM134A, FAM161A, FAM176B, FAM184A, FAM19A1, FAM20A, FAM23B, FAM65C, FANCA, FANCC, FANCG, FANCM, FANK1, FAR2, FBN1, FBXO15, FBXO18, FBXO38, FCGBP, FECH, FEZ2, FGA, FGD6, FGFR2, FGFR1OP, FGFR1OP2, FGFR2, FGG, FGR, FIX, FKBP3, FLI1, FLJ35848, FLJ36070, FLNA, FN1, FNBP1L, FOLH1, FOSL1, FOSL2, FOXK1, FOXM1, FOXO1, FOXP4, FRAS1, FUT9, FXN, FZD3, FZD6, GAB1, GABPA, GALC, GALNT3, GAPDH, GART, GAS2L3, GATA3, GATAD2A, GBA, GBGT1, GCG, GCGR, GCK, GFI1, GFM1, GH1, GHR, GHV, GJA1, GLA, GLT8D1, GNA11, GNAQ, GNAS, GNB5, GOLGB1, GOLT1A, GOLT1B, GPATCH1, GPR158, GPR160, GPX4, GRAMD3, GRHL1, GRHL2, GRHPR, GRIA1, GRIA3, GRIA4, GRIN2B, GRM3, GRM4, GRN, GSDMB, GSTCD, GSTO2, GTF2I, GTPBP4, HADHA, HAND2, HBA2, HBB, HCK, HDAC3, HDAC5, HDX, HEPACAM2, HERC1, HES7, HEXA, HEXB, HHEX, HIPK3, HLA-DPB1, HLA-G, HLCS, HLTF, HMBS, HMGA1, HMGCL, HNF1A, HNF1B, HNF4A, HNF4G, HNRNPH1, HOXC10, HP1BP3, HPGD, HPRT1, HPRT2, HSF1, HSF4, HSF2BP, HSPA9, HSPG2, HTT, HXA, ICA1, IDH1, IDS, IFI44L, IKBKAP, IKZF1, IKZF3, IL1R2, IL5RA, IL7RA, IMMT, INPP5D, INSR, INTS3, INTU, IP04, IP08, IQGAP2, IRF2, IRF4, IRF8, IRX3, ISL1, ISL2, ITFG1, ITGA6, ITGAL, ITGB1, ITGB2, 1TGB3, ITGB4, ITIHI, ITPR2, IWSI, JAK1, JAK2, JAG1, JMJD1C, JPH3, KALRN, KAT6A, KATNAL2, KCNN2, KCNT2, KDM2A, KIAA0256, KIAA0528, KIAA0564, KIAA0586, KIAA1033, KIAA1166, KIAA1219, KIAA1409, KIAA1622, KIAA1787, KIF3B, KIF15, KIF16B, KIF5A, KIF5B, KIF9, KIN, KIR2DL5B, KIR3DL2, KIR3DL3, KIT, KLF3, KLF5, KLF7, KLF10, KLF12, KLF16, KLHL20, KLK12, KLKB1, KMT2A, KMT2B, KPNA5, KRAS, KREMEN1, KRIT1, KRT5, KRTCAP2, KYNU, LICAM, L3MBTL, L3MBTL2, LACE1, LAMA1, LAMA2, LAMA3, LAMB1, LARP7, LDLR, LEF1, LENG1, LGALS3, LGMN, LHCGR, LHX3, LHX6, LIMCH1, LIMK2, LIN28B, LIN54, LMBRD1, LMBRD2, LMLN, LMNA, LMO2, LMO7, LOC389634, LOC390110, LPA, LPCAT2, LPL, LRP4, LRPPRC, LRRK2, LRRC19, LRRC42, LRWD1, LUM, LVRN, LYN, LYST, MADD, MAGI1, MAGT1, MALT1, MAP2K1, MAP4K4, MAPK8IP3, MAPK9, MAPT, MARC1, MARCH5, MATN2, MBD3, MCF2L2, MCM6, MDGA2, MDM4, ASXL1, FUS, SPR54, MECOM, MEF2C, MEF2D, MEGF10, MEGF11, MEMO1, MET, MGA, MGAM, MGAT4A, MGAT5, MGC16169, MGC34774, MKKS, MIB1, MIER2, MITF, MKL2, MLANA, MLH1, MLL5, MLX, MME, MPDZ, MPI, MRAP2, MRPL11, MRPL39, MRPS28, MRPS35, MS4A13, MSH2, MSH3, MSMB, MST1R, MTDH, MTERF3, MTF1, MTF2, MTIF2, MTHFR, MUC2, MUT, MVK, MYB, MYBL2, MYC, MYCBP2, MYH2, MYRF, MYT1, MY019, MY03A, MY09B, MYOM2, MYOM3, NAG, NARG1, NARG2, NCOA1, NDC80, NDFIP2, NEB, NEDD4, NEK1, NEK5, NEK11, NF1, NF2, NFATC2, NFE2L2, NFIA, NFIB, NFIX, NFKB1, NFKB2, NFKBIL2, NFRKB, NFYA, NFYB, NIPA2, NKAIN2, NKAP, NLRC3, NLRC5, NLRP3, NLRP7, NLRP8, NLRP13, NME1, NME1-NME2, NME2, NME7, NOL10, NOP561, NOS1, NOS2A, NOTCH1, NPAS4, NPM1, NRID1, NR1H3, NR1H4, NR4A3, NR5A1, NRXN1, NSMAF, NSMCE2, NT5C, NT5C2, NT5C3, NUBP1, NUBPL, NUDT5, NUMA1, NUP88, NUP98, NUP160, NUPL1, OAT, OAZ1, OBFC2A, OBFC2B, OLIG2, OMA1, OPA1, OPN4, OPTN, OSBPL11, OSBPL8, OSGEPL1, OTC, OTX2, OVOL2, OXT, PA2G4, PADI4, PAH, PAN2, PAOX, PAPOLG, PARD3, PARP1, PARVB, PAWR, PAX3, PAX8, PBGD, PBRM1, PBX2, PCBP4, PCCA, PCGF2, PCNX, PCOTH, PDCD4, PDE4D, PDE8B, PDE10A, PD1A3, PDH1, PDLIM5, PDXK, PDZRN3, PELI2, PDK4, PDS5A, PDS5B, PGK1, PGM2, PHACTR4, PHEX, PHKB, PHLDB2, PHOX2B, PHTFI, PIAS1, PIEZO1, PIGE, PIGN, PIGT, PIK3C2G, PIK3CA, PIK3CD, PIK3CG, PIK3RI, PIP5K1A, PITRM1, PIWIL3, PKD1, PKHD1L1, PKD2, PKIB, PKLR, PKM1, PKM2, PLAGL2, PLCB1, PLCB4, PLCG1, PLD1, PLEKHA5, PLEKHA7, PLEKHM1, PLKR, PLXNC1, PMFBP1, POLN, POLR3D, POMT2, POSTN, POU2AF1, POU2F2, POU2F3, PPARA, PPF1A2, PPP1R12A, PPP3CB, PPP4C, PPP4R1L, PPP4R2, PRAME, PRC1, PRDM1, PREX1, PREX2, PRIM1, PRIM2, PRKAR1A, PRKCA, PRKG1, PRMT7, PROC, PROCR, PROSC, PRODH, PROX1, PRPF40B, PRPF4B, PRRG2, PRUNE2, PSD3, PSEN1, PSMAL, PTCH1, PTEN, PTK2, PTK2B, PTPN2, PTPN3, PTPN4, PTPN11, PTPN22, PTPRD, PTPRK, PTPRM, PTPRN2, PTPRT, PUS10, PVRL2, PYGM, QRSL1, RAB11FIP2, RAB23, RAF1, RALBP1, RALGDS, RBICC1, RBL2, RBM39, RBM45, RBPJ, RBSN, REC8, RELB, RFC4, RFT1, RFTN1, RHOA, RHPN2, RIF1, RIT1, RLN3, RMND5B, RNF11, RNF32, RNFT1, RNGTT, ROCK1, ROCK2, RORA, RP1, RP6KA3, RP11-265F1, RP13-36C9, RPAP3, RPN1, RPGR, RPL22, RPL22L1, RPS6KA6, RREB1, RRM1, RRP1B, RSK2, RTEL1, RTF1, RUFY1, RUNX1, RUNX2, RXRA, RYR3, SAAL1, SAE1, SALL4, SAT1, SATB2, SBCAD, SCN1A, SCN2A, SCN3A, SCN4A, SCN5A, SCN8A, SCNA, SCN11A, SCO1, SCYL3, SDC1, SDK1, SDK2, SEC24A, SEC24D, SEC31A, SEL1L, SENP3, SENP6, SENP7, SERPINA1, SETD3, SETD4, SETDB1, SEZ6, SFRS12, SGCE, SGOL2, SGPL1, SH2D1A, SH3BGRL2, SH3PXD2A, SH3PXD2B, SH3RF2, SH3TC2, SHOC2, SIPA1L2, SIPA1L3, SIVA1, SKAP1, SKIV2L2, SLC6A11, SLC6A13, SLC6A6, SLC7A2, SLC12A3, SLC13A1, SLC22A17, SLC25A14, SLC28A3, SLC33A1, SLC35F6, SLC38A1, SLC38A4, SLC39A10, SLC4A2, SLC6A8, SMARCA1, SMARCA2, SMARCA5, SMARCC2, SMC5, SMN2, SMOX, SMS, SMTN, SNCA1P, SNORD86, SNRK, SNRP70, SNX5, SNX6, SOD1, SOD10, SOS, SOS2, SOX5, SOX6, SOX8, SP1, SP2, SP3, SP110, SPAG9, SPATA13, SPATA4, SPATS1, SPECC1L, SPDEF, SPI1, SPINK5, SPP2, SPTA1, SRF, SRM, SRP72, SSX3, SSX5, SSX9, STAG1, STAG2, STAMBPL1, STARD6, STAT1, STAT3, STAT5A, STAT5B, STAT6, STK17B, STX3, STXBP1, SUCLG2, SULF2, SUPT6H, SUPT16H, SV2C, SYCP2, SYT6, SYCP1, SYTL3, SYTL5, TAF2, TARDBP, TBC1D3G, TBC1D8B, TBC1D26, TBC1D29, TBCEL, TBK1, TBP, TBPL1, TBR1, TBX, TCEB3, TCF3, TCF4, TCF7L2, TCFL5, TCF12, TCP11L2, TDRD3, TEAD1, TEAD3, TEAD4, TECTB, TEK, TERF1, TERF2, TET2, TFAP2A, TFAP2B, TFAP2C, TFAP4, TFDP1, TFRC, TG, TGM7, TGS1, THAP7, THAP12, THOC2, TIAL1, TIAM2, TIMM50, TLK2, TM4SF20, TM6SF1, TMEM27, TMEM77, TMEM156, TMEM194A, TMF1, TMPRSS6, TNFRSF10A, TNFRSF10B, TNFRSF8, TNK2, TNKS, TNKS2, TOM1L1, TOM1L2, TOP2B, TP53, TP53INP1, TP53BP2, TP53I3, TP63, TRAF3IP3, TRAPPC2, TRIM44, TRIM65, TRIML1, TRIML2, TRPM3, TRPM5, TRPM7, TRPS1, TSC1, TSC2, TSHB, TSPAN7, TTC17, TTF1, TTLL5, TTLL9, TTN, TTPAL, TTR, TUSC3, TXNDC10, UBE3A, UCK1, UGT1A1, UHRF1BP1, UNC45B, UNC5C, USH2A, USF2, USP1, USP6, USP18, USP38, USP39, UTP20, UTP15, UTP18, UTRN, UTX, UTY, UVRAG, UXT, VAPA, VEGFA, VPS29, VPS35, VPS39, VT11A, VT11B, VWA3B, WDFY2, WDR16, WDR17, WDR26, WDR44, WDR67, WDTC1, WRN, WRNIP1, WT1, WWC3, XBP1, XRN1, XRN2, XX—FW88277, YAP1, YARS, YBX1, YGM, YY1, ZBTB18, ZBTB20, ZC3HAV1, ZC3HC1, ZC3H7A, ZDHHC19, ZEB1, ZEB2, ZFPM1, ZFYVE1, ZFX, ZIC2, ZNF37A, ZNF91, ZNF114, ZNF155, ZNF169, ZNF205, ZNF236, ZNF317, ZNF320, ZNF326, ZNF335, ZNF365, ZNF367, ZNF407, ZNF468, ZNF506, ZNF511, ZNF511-PRAP1, ZNF519, ZNF521, ZNF592, ZNF618, ZNF763, and ZWINT.

Additional exemplary genes encoding a target sequence (e.g., a target sequence comprising DNA or RNA, e.g., pre-mRNA) include genes include A1CF, A4GALT, AAR2, ABAT, ABCA1IP, ZNF721, ABCA5, ABHD10, ABHD13, ABHD2, ABHD6, AC000120.3, KRIT1, AC004076.1, ZNF772, AC004076.9, ZNF772, AC004223.3, RAD51D, AC004381.6, AC006486.1, ERF, AC007390.5, AC007780.1, PRKAR1A, AC007998.2, INO80C, AC009070.1, CMC2, AC009879.2, AC009879.3, ADHFE1, AC010487.3, ZNF816-ZNF321P, ZNF816, AC010328.3, AC010522.1, ZNF587B, AC010547.4, ZNF19, AC012313.3, ZNF497, AC012651.1, CAPN3, AC013489.1, DET1, AC016747.4, C2orf74, AC020907.6, FXYD3, AC021087.5, PDCD6, AHRR, AC022137.3, ZNF761, AC025283.3, NAA60, AC027644.4, RABGEF1, AC055811.2, FLCN, AC069368.3, ANKDD1A, AC073610.3, ARF3, AC074091.1, GPN1, AC079447.1, LIPT1, AC092587.1, AC079594.2, TRIM59, AC091060.1, C18orf21, AC092143.3, MC1R, AC093227.2, ZNF607, AC093512.2, ALDOA, AC098588.1, ANAPC10, AC107871.1, CALML4, AC114490.2, ZMYM6, AC138649.1, NIPA1, AC138894.1, CLN3, AC139768.1, AC242426.2, CHD1L, ACADM, ACAP3, ACKR2, RP11-141M3.5, KRBOX1, ACMSD, ACOT9, ACP5, ACPL2, ACSBG1, ACSF2, ACSF3, ACSL1, ACSL3, ACVR1, ADAL, ADAM29, ADAMTS10, ADAMTSL5, ADARB1, ADAT2, ADCK3, ADD3, ADGRG1, ADGRG2, ADH1B, ADIPOR1, ADNP, ADPRH, AGBL5, AGPAT1, AGPAT3, AGR2, AGTR1, AHDC1, AHI1, AHNAK, AIFM1, AIFM3, AIMP2, AK4, AKAP1, AKNAD1, CLCC1, AKRIA1, AKT1, AKTIS1, AKT2, AL139011.2, PEX19, AL157935.2, ST6GALNAC6, AL358113.1, TJP2, AL441992.2, KYAT1, AL449266.1, CLCC1, AL590556.3, LINC00339, CDC42, ALAS1, ALB, ALDH16A1, ALDH1B1, ALDH3A1, ALDH3B2, ALDOA, ALKBH2, ALPL, AMD1, AMICA1, AMN1, AMOTL2, AMY1B, AMY2B, ANAPC10, ANAPC11, ANAPC15, ANG, RNASE4, AL163636.2, ANGEL2, ANGPTL1, ANKMY1, ANKRD11, ANKRD28, ANKRD46, ANKRD9, ANKS3, ANKS3, RP11-127120.7, ANKS6, ANKZF1, ANPEP, ANXA11, ANXA2, ANXA8L2, AL603965.1, AOC3, AP000304.12, CRYZL1, AP000311.1, CRYZL1, AP000893.2, RAB30, AP001267.5, ATP5 MG, AP002495.2, AP003175.1, OR2AT4, AP003419.1, CLCF1, AP005263.1, ANKRD12, AP006621.5, AP006621.1, AP1G1, AP3M1, AP3M2, APBA2, APBB1, APLP2, APOA2, APOL1, APOL3, APTX, ARAP1, STARD10, ARF4, ARFIP1, ARFIP2, ARFRP1, ARHGAP11A, ARHGAP33, ARHGAP4, ARHGEF10, ARHGEF3, ARHGEF35, OR2A1-AS1, ARHGEF35, OR2A1-AS1, ARHGEF34P, ARID1B, ARHGEF35, OR2A20P, OR2A1-AS1, ARHGEF9, ARL1, ARL13B, ARL16, ARL6, ARMC6, ARMC8, ARMCX2, ARMCX5, RP4-769N13.6, ARMCX5-GPRASP2, BHLHB9, ARMCX5-GPRASP2, GPRASP1, ARMCX5-GPRASP2, GPRASP2, ARMCX6, ARNT2, ARPP19, ARRB2, ARSA, ART3, ASB3, GPR75-ASB3, ASCC2, ASNS, ASNS, AC079781.5, ASPSCR1, ASS1, ASUN, ATE1, ATF1, ATF7IP2, ATG13, ATG4D, ATG7, ATG9A, ATM, ATOX1, ATP1B3, ATP2C1, ATP5F1A, ATP5G2, ATP5J, ATP5MD, ATP5 PF, ATP6AP2, ATP6V0B, ATP6V1C1, ATP6V1D, ATP7B, ATXN1, ATXN1L, IST1, ATXN3, ATXN7L1, AURKA, AURKB, AXDND1, B3GALNT1, B3GALT5, AF064860.1, B3GALT5, AF064860.5, B3GNT5, B4GALT3, B4GALT4, B9D1, BACH1, BAIAP2, BANF1, BANF2, BAX, BAZ2A, BBIP1, BCHE, BCL2L14, BCL6, BCL9L, BCS1L, BDH1, BDKRB2, AL355102.2, BEST1, BEST3, BEX4, BHLHB9, BID, BIN3, BIRC2, BIVM, BIVM-ERCC5, BIVM, BLCAP, BLK, BLOC1S1, RP11-644F5.10, BLOC1S6, AC090527.2, BLOC1S6, RP11-96020.4, BLVRA, BMF, BOLA1, BORCS8-MEF2B, BORCS8, BRCA1, BRD1, BRDT, BRINP3, BROX, BTBD10, BTBD3, BTBD9, BTD, BTF3L4, BTNL9, BUB1B-PAK6, PAK6, BUB3, C10orf68, C11orf1, C11orf48, C11orf54, C11orf54, AP001273.2, C11orf57, C11orf63, C11orf82, C12orf23, C12orf4, C12orf65, C12orf79, C14orf159, C14orf93, C17orf62, C18orf21, C19orf12, C19orf40, C19orf47, C19orf48, C19orf54, C1D, C1GALT1, C1QB, C1QTNF1, C1S, C1orf101, C1orf112, C1orf116, C1orf159, C1orf63, C2, C2, CFB, C20orf27, C21orf58, C2CD4D, C2orf15, LIPT1, MRPL30, C2orf80, C2orf81, C3orf14, C3orf17, C3orf18, C3orf22, C3orf33, AC104472.3, C4orf33, C5orf28, C5orf34, C1orf118, C6orf203, C6orf211, C6orf48, C7orf50, C7orf55, C7orf55-LUC7L2, LUC7L2, C8orf44-SGK3, C8orf44, C8orf59, C9, DAB2, C9orf153, C9orf9, CA5BP1, CA5B, CABYR, CALCA, CALCOCO1, CALCOCO2, CALM1, CALM3, CALML4, RP11-315D16.2, CALN1, CALU, CANT1, CANX, CAP1, CAPN12, CAPS2, CARD8, CARHSP1, CARNS1, CASC1, CASP3, CASP7, CBFA2T2, CBS, CBY1, CCBL1, CCBL2, RBMXL1, CCDCl2, CCDCl26, CCDCl4, CCDCl49, CCDCl50, CCDCl69-SOHLH2, CCDCl69, CCDCl71, CCDC37, CCDC41, CCDC57, CCDC63, CCDC7, CCDC74B, CCDC77, CCDC82, CCDC90B, CCDC91, CCDC92, CCNE1, CCHCR1, CCL28, CCNB11P1, CCNC, CCND3, CCNG1, CCP110, CCR9, CCT7, CCT8, CD151, CD1D, CD200, CD22, CD226, CD276, CD36, CD59, CDC26, CDC42, CDC42SE1, CDC42SE2, CDHR3, CDK10, CDK16, CDK4, CDKAL1, CDKL3, CTD-2410N18.4, CDKN1A, CDKN2A, CDNF, CEBPZOS, CELF1, CEMIP, CENPK, CEP170B, CEP250, CEP57, CEP57L1, CEP63, CERS4, CFL1, CFL2, CFLAR, CGNL1, CHCHD7, CHD1L, CHD8, CHFR, ZNF605, CHIA, CHID1, CHL1, CHM, CHMP1A, CHMP3, RNF103-CHMP3, CHRNA2, C1DEC, C1RBP, CITED1, CKLF-CMTM1, CMTM1, CKMT1B, CLDN12, CTB-13L3.1, CLDND1, AC021660.3, CLDND1, CPOX, CLHC1, CLIP1, CLUL1, CMC4, MTCP1, CNDP2, CNFN, CNOT1, CNOT6, CNOT7, CNOT8, CNR1, CNR2, CNTFR, CNTRL, COA1, COASY, COCH, COL8A1, COLCA1, COLEC11, COMMD3-BMI1, BMI1, COPS5, COPS7B, COQ8A, CORO6, COTL1, COX14, RP4-60503.4, COX7A2, COX7A2L, COX7B2, CPA4, CPA5, CPEB1, CPNE1, AL109827.1, RBM12, CPNE1, RP1-309K20.6, RBM12, CPNE3, CPSF3L, CPT1C, CREB3L2, CREM, CRP, CRYZ, CS, AC073896.1, CS, RP11-977G19.10, CSAD, CSDE1, CSF2RA, CSGALNACT1, CSK, CSNK2A1, CSRNP2, CT45A4, CT45A4, CT45A5, CT45A6, CTBP2, CTCFL, CTD-2116N17.1, KIAA0101, CTD-2349B8.1, SYT17, CTD-2528L19.4, ZNF607, CTD-2619J13.8, ZNF497, CTNNA1, CTNNBIP1, CTNND1, CTPS2, CTSB, CTSL, CTTN, CUL2, CUL9, CWC15, CXorf40B, CYB561A3, CYBC1, CYLD, CYP11A1, CYP2R1, CYP4B1, CYP4F22, DAG1, DAGLB, KDELR2, DARS, DBNL, DCAF11, DCAF8, PEX19, DCLRE1C, DCTD, DCTN1, DCTN4, DCUN1D2, DDR1, DDX11, DDX19B, AC012184.2, DDX19B, RP11-529K1.3, DDX25, DDX39B, ATP6V1G2-DDX39B, SNORD84, DDX42, DDX60L, DEDD, DEDD2, DEFA1, DEFA1B, DEFA1B, DEFA3, DENND1C, DENND2A, DENND4B, DET1, DGKA, DGKZ, DGLUCY, DHRS4L2, DHRS9, DHX40, DIABLO, AC048338.1, DIAPH1, DICER1, DKKL1, DLG1, DLG3, DLST, DMC1, DMKN, DMTF1, DMTN, DNAJC14, DNAJC19, DNAL1, DNASE1L1, DNMT3A, DOC2A, DOCK8, DOK1, DOPEY1, DPAGT1, DPP8, DRAM2, DRD2, DROSHA, DSN1, DTNA, DTX2, DTX3, DUOX1, DUOXA1, DUS2, DUSP10, DUSP13, DUSP18, DUSP22, DYDC1, DYDC2, DYNLL1, DYNLT1, DYRK1A, DYRK2, DYRK4, RP11-500M8.7, DZIP1L, E2F6, ECHDC1, ECS1T, ECT2, EDC3, EDEM1, EDEM2, MMP24-AS1, RP4-61404.11, EEF1AKNMT, EEF1D, EFEMP1, EFHC1, EGFL7, EHF, EI24, EIF1AD, EIF2B5, EIF4G1, EIF2B5, POLR2H, EIF3E, EIF3K, EIF4E3, EIF4G1, ELF1, ELMO2, ELMOD1, AP000889.3, ELMOD3, ELOC, ELOF1, ELOVL1, ELOVL7, ELP1, ELP6, EML3, EMP3, ENC1, ENDOV, ENO1, ENPP5, ENTHD2, ENTPD6, EP400NL, EPB41L1, EPDR1, NME8, EPHX1, EPM2A, EPN1, EPN2, EPN3, EPS8L2, ERBB3, ERC1, ERCC1, ERG, ERI2, ERI2, DCUN1D3, ERL1N2, ERMARD, ERRF11, ESR2, RP11-544I20.2, ESRRA, ESRRB, ESRRG, ETFA, ETFRF1, ETV1, ETV4, ETV7, EVA1A, EVC2, EVX1, EXD2, EXO5, EXOC1, EXOC2, FAAP24, FABP6, FADS1, FADS2, FAHD2B, FAM107B, FAM111A, FAM111B, FAM114A1, FAM114A2, FAM115C, FAM115C, FAM115D, FAM120B, FAM133B, FAM135A, FAM153A, FAM153B, FAM154B, FAM156A, FAM156B, FAM168B, FAM172A, FAM182B, FAM192A, FAM19A2, FAM200B, FAM220A, FAM220A, AC009412.1, FAM222B, FAM227B, FAM234A, AC004754.1, FAM3C, FAM45A, FAM49B, FAM60A, FAM63A, FAM81A, FAM86B1, FAM86B2, FANC1, FANK1, FAR2, FAXC, FAXDC2, FBF1, FBH1, FBXL4, FBXO18, FBXO22, FBXO31, FBXO41, FBXO44, FBXO45, FBXW9, FCHO1, FCHSD2, FDFT1, FDPS, FER, FETUB, FGD4, FGF1, FGFR1, FGFRL1, FGL1, FHL2, FIBCD1, FIGNL1, FIGNL1, DDC, FKBP5, FKRP, FLRT2, FLRT3, FMC1, LUC7L2, FMC1-LUC7L2, FNDC3B, FOLH1, FOLR1, FOXP1, FOXK1, FOXM1, FOXO1, FOXP4, AC097634.4, FOXRED1, FPR1, FPR2, FRG1B, FRS2, FTO, FTSJ1, FUK, FUT10, FUT3, FUT6, FXYD3, FZD3, G2E3, GAA, GABARAPL1, GABPB1, GABRA5, GAL3ST1, GALE, GALNT11, GALNT14, GALNT6, GAPVD1, GARNL3, GAS2L3, GAS8, GATA1, GATA2, GATA4, GBA, GCNT1, GDPD2, GDPD5, GEMIN7, MARK4, GEMIN8, GGA3, GGACT, AL356966.1, GGPS1, GHRL, GID8, GIGYF2, GIMAP8, GIPC1, GJB1, GJB6, GLB1L, GLI1, GLT8D1, GMFG, GMPR2, GNAI2, GNAQ, GNB1, GNB2, GNE, GNG2, GNGT2, GNPDA1, GNPDA2, GOLGA3, CHFR, GOLGA4, GOLPH3L, GOLT1B, GPBP1L1, GPER1, GPR116, GPR141, EPDR1, GPR155, GPR161, GPR56, GPR63, GPR75-ASB3, ASB3, GPR85, GPSM2, GRAMD1B, GRB10, GRB7, GREM2, GRIA2, GSDMB, GSE1, GSN, GSTA4, GSTZ1, GTDC1, GTF2H1, GTF2H4, VARS2, GTF3C2, GUCY1A3, GUCY1B3, GUK1, GULP1, GYPC, GYS1, GZF1, HAGH, HAO2, HAPLN3, HAVCR, HAX1, HBG2, AC104389.4, HBG2, AC104389.4, HBE1, HBG2, AC104389.4, HBE1, OR51B5, HBG2, HBE1, AC104389.28, HBS1L, HCFC1R1, HCK, HDAC2, HDAC6, HDAC7, HDLBP, HEATR4, HECTD4, HEXIM2, HHAT, HHATL, CCDC13, HINFP, HIRA, C22orf39, HIVEP3, HJV, HKR1, HLF, HMBOX1, HMGA1, HMGB3, HMGCR, HMGN4, HMOX2, HNRNPC, HNRNPD, HNRNPH1, HNRNPH3, HNRNPR, HOMER3, HOPX, HOXA3, HOXB3, HOXB3, HOXB4, HOXC4, HOXD3, HOXD3, HOXD4, HPCAL1, HPS4, HPS5, HRH1, HS3ST3A1, HSH2D, HSP90AA1, HSPD1, HTT, HUWE1, HYOU1, IAH1, ICA1L, ICAM2, ICE2, ICK, IDH2, IDH3G, IDS, IFI27, IFI44, IFT20, IFT22, IFT88, IGF2, INS-IGF2, IGF2BP3, IGFBP6, IKBKAP, IKBKB, IL11, IL18BP, IL18RAP, IL1RAP, IL1RL1, IL18R1, IL1RN, IL32, IL411, NUP62, AC011452.1, IL411, NUP62, CTC-326K19.6, IL6ST, ILVBL, IMMP1L, IMPDH1, INCA1, ING1, IN1P, INPP1, INPP5J, INPP5K, INSIG2, INTS11, INTS12, INTS14, IP6K2, IP6K3, IPO11, LRRC70, IQCE, IQGAP3, IRAK4, IRF3, IRF5, IRF6, ISG20, IST1, ISYNA1, ITFG2, ITGB1BP1, ITGB7, ITIH4, RP5-966M1.6, ITPR1PL1, JADE1, JAK2, JARID2, JDP2, KANK1, KANK1, RP11-31F19.1, KANK2, KANSL1L, KAT6A, KBTBD2, KBTBD3, KCNAB2, KCNE3, KCNG1, KCNJ16, KCNJ9, KCNMB2, AC117457.1, LINC01014, KCTD20, KCTD7, RABGEF1, KDM1B, KDM4A, AL451062.3, KHNYN, KIAA0040, KIAA0125, KIAA0196, KIAA0226L, PPP1R2P4, KIAA0391, KIAA0391, AL121594.1, KIAA0391, PSMA6, KIAA0753, KIAA0895, KIAA0895L, KIAA1191, KIAA1407, KIAA1841, C2orf74, KIF12, KIF14, KIF27, KIF9, KIFC3, KIN, KIRREL1, KITLG, KLC1, APOPT1, AL139300.1, KLC4, KLHDC4, KLHDC8A, KLHL13, KLHL18, KLHL2, KLHL24, KLHL7, KLK11, KLK2, KLK5, KLK6, KLK7, KNOP1, KRBA2, AC135178.2, KRBA2, RP11-849F2.7, KRIT1, KRT15, KRT8, KTN1, KXD1, KYAT3, RBMXL1, KYNU, L3MBTL1, LACC1, LARGE, LARP4, LARP7, LAT2, LBHD1, LCA5, LCA5L, LCTL, LEPROTL1, LGALS8, LGALS9C, LGMN, LHFPL2, LIG4, LIMCH1, LIMK2, LIMS2, LINC00921, ZNF263, LIPF, LLGL2, LMAN2L, LMCD1, LMF1, RP11-161M6.2, LMO1, LMO3, LOXHD1, LPAR1, LPAR2, LPAR4, LPAR5, LPAR6, LPHN1, LPIN2, LPIN3, LPP, LRFN5, LRIF1, LRMP, LRRC14, LRRC20, LRRC24, C8orf82, LRRC39, LRRC42, LRRC48, LRRC4C, LRRC8A, LRRC8B, LRRD1, LRTOMT, LRTOMT, AP000812.5, LSM7, LTB4R, LTBP3, LUC7L2, FMC1-LUC7L2, LUC7L3, LUZP1, LYG1, LYL1, LYPD4, LYPD6B, LYRM1, LYRM5, LYSMD4, MACC1, MAD1L1, MAD1L1, AC069288.1, MAEA, MAFF, MAFG, MAFK, MAGEA12, CSAG4, MAGEA2, MAGEA2B, MAGEA4, MAGEB1, MAGOHB, MAN2A2, MANBAL, MAOB, MAP2K3, MAP3K7CL, MAP3K8, MAP7, MAP9, MAPK6, MAPK7, MAPK8, MAPKAP1, 10-Mar, 7-Mar, 8-Mar, MARK2, MASP1, MATK, MATR3, MATR3, SNHG4, MB, MBD5, MBNL1, MBOAT7, MCC, MCFD2, MCM9, MCOLN3, MCRS1, MDC1, MDGA2, MDH2, MDM2, ME1, MEAK7, MECR, MED4, MEF2A, MEF2B, BORCS8-MEF2B, MEF2BNB-MEF2B, MEF2B, MEF2BNB, MEF2C, MEF2D, MEGF10, MEI1, MEIS2, MELK, MET, METTL13, METTL23, MFF, MEN2, MFSD2A, MGST3, MIB2, MICAL1, MICAL3, MICOS10, NBL1, MICOS10-NBL1, MID1, MINA, MINOS1-NBL1, MINOS1, MIOS, MIPOL1, MIS12, MKLN1, MKNK1, MKNK1, MOB3C, MLF2, MLH1, MMP17, MOBP, MOCS1, MOGS, MOK, MORF4L1, MPC1, MPC2, MPG, MPI, MPP1, MPP2, MPPE1, MPST, MRAS, MRO, MROH1, MROH7-TTC4, MROH7, MRPL14, MRPL24, MRPL33, BABAM2, MRPL33, BRE, MRPL47, MRPL48, MRPL55, MRRF, MRTFA, MRTFB, MRVI1, MS4A1, MS4A15, MS4A3, MS4A6E, MS4A7, MS4A14, MSANTD3, MSANTD4, MSH5, MSH5-SAPCD1, MSL2, MSRB3, MSS51, MTCP1, CMC4, MTERF, MTERF1, MTERF3, MTERFD2, MTERFD3, MTF2, MTG2, MTHFD2, MTHFD2L, MTIF2, MTIF3, MTMR10, MTRF1, MTRR, MTUS2, MUTYH, MVK, MX1, MX2, MYH10, MYL12A, MYB, MYD88, MYL5, MYL1P, MYNN, MYO15A, MYO1B, MYOM2, MZF1, N4BP2L2, NAA60, NAB1, NAE1, NAGK, NAP1L1, NAP IL4, NAPG, NARFL, NARG2, NAT1, NAT10, NBPF11, WI2-3658N16.1, NBPF12, NBPF15, NBPF24, NBPF6, NBPF9, NBR1, NCAPG2, NCBP2, NCEH1, NCOA1, NCOA4, NDC1, NDRG1, NDRG2, NDRG4, NDST1, NDUFAF6, NDUFB2, NDUFC1, NDUFS1, NDUFS8, NDUFV1, NEDD1, NEIL1, NEIL2, NEK10, NEK11, NEK6, NEK9, NELFA, NEU4, NFAT5, NFE2, NFE2L2, AC019080.1, NFRKB, NFYA, NFYC, NIF3L1, NIPA2, NKIRAS1, NKX2-1, NLRC3, NME1, NME1-NME2, NME2, NME1-NME2, NME2, NME4, NME6, NME9, NOD1, NOL10, NOL8, NONO, NPAS1, NPIPA8, RP11-1212A22.1, NPIPB3, NPIPB4, NPIPB9, NPL, NPM1, NPPA, NQO2, NR1H3, NR2C2, NR2F2, NR4A1, NRDC, NREP, NRF1, NRG4, NRIP1, NSD2, NSDHL, NSG1, NSMCE2, NSRP1, NT5C2, NTF4, NTMT1, NTNG2, NUBP2, NUCB2, NUDT1, NUDT2, NUDT4, NUF2, NUMBL, NUP50, NUP54, NUP85, NVL, NXF1, NXPE1, NXPE3, OARD1, OAT, OAZ2, OCIAD1, OCLN, ODF2, OGDHL, OGFOD2, AC026362.1, OGFOD2, RP11-197N18.2, OLA1, OPRL1, OPTN, OR2H1, ORA12, ORMDL1, ORMDL2, ORMDL3, OSBPL2, OSBPL3, OSBPL5, OSBPL9, OSER1, OSGIN1, OSR2, P2RX4, P2RY2, P2RY6, P4HA2, PABPC1, PACRGL, PACSIN3, PADI1, PAIP2, PAKI, PAK3, PAK4, PAK7, PALB2, PANK2, PAQR6, PARP11, PARVG, PASK, PAX6, PBRM1, PBXIP1, PCBP3, PCBP4, AC115284.1, PCBP4, RP11-155D18.14, RP11-155D18.12, PCGF3, PCGF5, PCNP, PCSK9, PDCD10, PDCD6, AHRR, PDDC1, PDGFRB, PDIA6, PDIK1L, PDLIM7, PDP1, PDPK1, PDPN, PDZD11, PEA15, PEX2, PEX5, PEX5L, PFKM, PFN4, PGAP2, PGAP2, AC090587.2, PGAP3, PGM3, PGPEP1, PHB, PHC2, PHF20, PHF21A, PHF23, PHKB, PHLDB1, PHOSPHO1, PHOSPHO2, KLHL23, PI4 KB, PIAS2, PICALM, PIF1, PIGN, PIGO, PIGT, PIK3CD, PILRB, STAG3L5P-PVRIG2P-PILRB, PIP5K1B, PIR, PISD, PIWIL4, FUT4, PKD2, PKIA, PKIG, PKM, PKN2, PLAIA, PLA2G2A, PLA2G5, PLA2G7, PLAC8, PLAGL1, PLD1, PLD3, PLEKHA1, PLEKHA2, PLEKHA6, PLEKHG5, PLIN1, PLS1, PLS3, PLSCR1, PLSCR2, PLSCR4, PLXNB1, PLXNB2, PMP22, PMS1, PNISR, PNKP, AKTIS1, PNMT, PNPLA4, PNPLA8, PNPO, PNRC1, POC1B, POFUT1, POLB, POLD1, POLH, POLI, POLL, POLR1B, POM121, POM121C, AC006014.7, POM121C, AC211429.1, POMC, POMT1, POP1, PORCN, POU5F1, PSORSIC3, PPARD, PPARG, PPHLN1, PPIL3, PPIL4, PPM1A, PPM1B, AC013717.1, PPP1CB, PPP1R11, PPP1R13L, PPP1R26, PPP1R9A, PPP2R2B, PPP3CA, PPP6R1, PPP6R3, PPT2, PPT2-EGFL8, EGFL8, PPWD1, PRDM2, PRDM8, PRELID3A, PREPL, PRICKLE1, PRKAG1, PRMT2, PRMT5, PRMT7, PROM1, PRPS1, PRPSAP2, PRR14L, PRR15L, PRR5, PRR5-ARHGAP8, PRR5L, PRR7, PRRC2B, PRRT4, PRSS50, PRSS45, PRSS44, PRUNE, PRUNE1, PSEN1, PSMA2, PSMF1, PSORS1C1, PSPH, PSRC1, PTBP3, PTHLH, PTK2, PTPDC1, PTPRM, PUF60, PUM2, PUS1, PUS10, PXN, PXYLP1, PYCR1, QRICH1, R3HCC1L, R3HDM2, RAB17, RAB23, RAB3A, RAB3D, TMEM205, RAB4B-EGLN2, EGLN2, AC008537.1, RAB5B, RAB7L1, RABL2A, RABL2B, RABL5, RACGAP1, RAD17, RAD51L3-RFFL, RAD51D, RAD52, RAEI, RAI14, RAI2, RALBP1, RAN, RANGAP1, RAP1A, RAP1B, RAP1GAP, RAPGEF4, RAPGEFL1, RASGRP2, RASSF1, RBCK1, RBM12B, RBM14, RBM4, RBM14-RBM4, RBM23, RBM4, RBM14-RBM4, RBM47, RBM7, AP002373.1, RBM7, RP11-212D19.4, RBMS2, RBMY1E, RBPJ, RBPMS, RBSN, RCBTB2, RCC1, RCC1, SNHG3, RCCD1, RECOL, RELL2, REPIN1, AC073111.3, REPIN1, ZNF775, RER1, RERE, RFWD3, RFX3, RGL2, RGMB, RGS11, RGS3, RGS5, AL592435.1, RHBDD1, RHNO1, TULP3, RHOC, AL603832.3, RHOC, RP11-426L16.10, RHOH, RIC8B, RIMKLB, RIN1, RIPK2, RIT1, RLIM, RNASE4, ANG, AL163636.6, RNASEK, RNASEK-C17orf49, RNF111, RNF123, RNF13, RNF14, RNF185, RNF216, RNF24, RNF32, RNF34, RNF38, RNF4, RNF44, RNH1, RNMT, RNPS1, RO60, ROPN1, ROPN1B, ROR2, RP1-102H19.8, C6orf163, RP1-283E3.8, CDK11A, RP11-120M18.2, PRKAR1A, RP11-133K1.2, PAK6, RP11-164J13.1, CAPN3, RP11-21J18.1, ANKRD12, RP11-322E11.6, INO80C, RP11-337C18.10, CHD1L, RP11-432B6.3, TRIM59, RP11-468E2.4, IRF9, RP11-484M3.5, UPK1B, RP11-517H2.6, CCR6, RP11-613M10.9, SLC25A51, RP11-659G9.3, RAB30, RP11-691N7.6, CTNND1, RP11-849H4.2, RP11-896J10.3, NKX2-1, RP11-96020.4, SQRDL, RP11-986E7.7, SERPINA3, RP4-769N13.6, GPRASP1, RP4-769N13.6, GPRASP2, RP4-798P15.3, SEC16B, RP5-1021I20.4, ZNF410, RP6-109B7.3, FLJ27365, RPE, RPH3AL, RPL15, RPL17, RPL17-C18orf32, RPL17, RPL23A, RPL36, HSD11B1L, RPP38, RPS20, RPS27A, RPS3A, RPS6KA3, RPS6KC1, RPS6KL1, RPUSD1, RRAGD, RRAS2, RRBP1, RSL1D1, RSRC2, RSRP1, RUBCNL, RUNX1T1, RUVBL2, RWDD1, RWDD4, S100A13, AL162258.1, S100A13, RP1-178F15.5, S100A16, S100A4, S100A3, S100A6, S100PBP, SAA1, SACM1L, SAMD4B, SAR1A, SARAF, SARNP, RP11-76217.5, SCAMP5, SCAP, SCAPER, SCFD1, SCGB3A2, SC1N, SCML1, SCNN1D, SCO2, SCOC, SCRN1, SDC2, SDC4, SEC13, SEC14L1, SEC14L2, SEC22C, SEC23B, SEC24C, SEC61G, SEMA4A, SEMA4C, SEMA4D, SEMA6C, SENP7, SEPP1, 11-Sep, 2-Sep, SERGEF, AC055860.1, SERP1, SERPINA1, SERPINA5, SERPINB6, SERPING1, SERPINH1, SERTAD3, SETD5, SFMBT1, AC096887.1, SFTPA1, SFTPA2, SFXN2, SGCD, SGCE, SGK3, SGK3, C8orf44, SH2B1, SH2D6, SH3BP1, Z83844.3, SH3BP2, SH3BP5, SH3D19, SH3YL1, SHC1, SHISA5, SHMT1, SHMT2, SHOC2, SHROOM1, SIGLEC5, SIGLEC14, SIL1, SIN3A, SIRT2, SIRT6, SKP1, STAT4, AC104109.3, SLAIN1, SLC10A3, SLC12A9, SLC14A1, SLC16A6, SLC1A2, SLC1A6, SLC20A2, SLC25A18, SLC25A19, SLC25A22, SLC25A25, SLC25A29, SLC25A30, SLC25A32, SLC25A39, SLC25A44, SLC25A45, SLC25A53, SLC26A11, SLC26A4, SLC28A1, SLC29A1, SLC2A14, SLC2A5, SLC2A8, SLC35B2, SLC35B3, SLC35C2, SLC37A1, SLC38A1, SLC38A11, SLC39A13, SLC39A14, SLC41A3, SLC44A3, SLC4A7, SLC4A8, SLC5A10, SLC5A11, SLC6A1, SLC6A12, SLC6A9, SLC7A2, SLC7A6, SLC7A7, SLCO1A2, SLCO1C1, SLCO2B1, SLFN11, SLFN12, SLFNL1, SLMO1, SLTM, SLU7, SMAD2, SMAP2, SMARCA2, SMARCE1, AC073508.2, SMARCE1, KRT222, SMC6, SMG7, SMIM22, SMOX, SMPDL3A, SMTN, SMU1, SMUG1, SNAP25, SNCA, SNRK, SNRPC, SNRPD1, SNRPD2, SNRPN, SNRPN, SNURF, SNUPN, SNX11, SNX16, SNX17, SOAT1, SOHLH2, CCDCl69-SOHLH2, CCDC169, SORBS1, SORBS2, SOX5, SP2, SPART, SPATA20, SPATA21, SPATS2, SPATS2L, SPDYE2, SPECC1, SPECC1L, SPECC1L-ADORA2A, SPECC1L-ADORA2A, ADORA2A, SPEG, SPG20, SPG21, SPIDR, SPIN1, SPOCD1, SPOP, SPRR2A, SPRR2B, SPRR2E, SPRR2B, SPRR2F, SPRR2D, SPRR3, SPRY1, SPRY4, SPTBN2, SRC, SRGAP1, SRP68, SRSF11, SSX1, SSX2IP, ST3GAL4, ST3GAL6, ST5, ST6GALNAC6, ST7L, STAC3, STAG1, STAG2, STAMBP, STAMBPL1, STARD3NL, STAT6, STAU1, STAU2, AC022826.2, STAU2, RP11-463D19.2, STEAP2, STEAP3, ST1L, STK25, STK33, STK38L, STK40, STMN1, STON1, STON1-GTF2A1L, STRAP, STRBP, STRC, AC011330.5, STRC, CATSPER2, STRC, CATSPER2, AC011330.5, STRC, STRCP1, STT3A, STX16-NPEPL1, NPEPL1, STX5, STX6, STX8, STXBP6, STYK1, SULT1A1, SULT1A2, SUMF2, SUN1, SUN2, SUN2, DNAL4, SUOX, SUPT6H, SUV39H2, SV2B, SYBU, SYNCR1P, SYNJ2, SYT1, SYTL4, TAB2, TACC1, TADA2B, TAF1C, TAF6, AC073842.2, TAF6, RP11-506M12.1, TAF9, TAGLN, TANK, TAPSAR1, PSMB9, TAPT1, TATDN1, TAZ, TBC1D1, TBC1D12, HELLS, TBC1D15, TBC1D3H, TBC1D3G, TBC1D5, TBC1D5, SATB1, TBCA, TBCEL, TBCEL, AP000646.1, TBL1XR1, TBP, TBX5, TBXAS1, TCAF1, TCEA2, TCEAL4, TCEAL8, TCEAL9, TCEANC, TCEB1, TCF19, TCF25, TCF4, TCP1, TCP10L, AP000275.65, TCP11, TCP11L2, TCTN1, TDG, TDP1, TDRD7, TEAD2, TECR, TENC1, TENT4A, TEX264, TEX30, TEX37, TFDP1, TFDP2, TFEB, TFG, TFP1, TF, TFP1, TGIF1, THAP6, THBS3, THOC5, THRAP3, THUMPD3, TIAL1, TIMM9, TIMP1, TIRAP, TJAP1, TJP2, TK2, TLDC1, TLE3, TLE6, TLN1, TLR10, TM9SF1, TMBIM1, TMBIM4, TMBIM6, TMC6, TMCC1, TMCO4, TMEM126A, TMEM139, TMEM150B, TMEM155, TMEM161B, TMEM164, TMEM168, TMEM169, TMEM175, TMEM176B, TMEM182, TMEM199, CTB-96E2.3, TMEM216, TMEM218, TMEM230, TMEM263, TMEM45A, TMEM45B, TMEM62, TMEM63B, TMEM66, TMEM68, TMEM98, TMEM9B, TMPRSS11D, TMPRSS5, TMSB15B, TMTC4, TMUB2, TMX2-CTNND1, RP11-691N7.6, CTNND1, TNFAIP2, TNFAIP8L2, SCNM1, TNFRSF10C, TNFRSF19, TNFRSF8, TNFSF12-TNFSF13, TNFSF12, TNFSF13, TNFSF12-TNFSF13, TNFSF13, TNIP1, TNK2, TNNT1, TNRC18, TNS3, TOB2, TOM1L1, TOP1MT, TOP3B, TOX2, TP53, RP11-199F11.2, TP53I11, TP53INP2, TPCN1, TPM3P9, AC022137.3, TPT1, TRA2B, TRAF2, TRAF3, TRAPPC12, TRAPPC3, TREH, TREX1, TREX2, TRIB2, TRIM3, TRIM36, TRIM39, TRIM46, TRIM6, TRIM6-TRIM34, TRIM6-TRIM34, TRIM34, TRIM66, TRIM73, TRIT1, TRMT10B, TRMT2B, TRMT2B-AS1, TRNT1, TRO, TROVE2, TRPS1, TRPT1, TSC2, TSGA10, TSPAN14, TSPAN3, TSPAN4, TSPAN5, TSPAN6, TSPAN9, TSPO, TTC12, TTC23, TTC3, TTC39A, TTC39C, TTLL1, TTLL7, TTPAL, TUBD1, TWNK, TXNL4A, TXNL4B, TXNRD1, TYK2, U2AF1, UBA2, UBA52, UBAP2, UBE2D2, UBE2D3, UBE2E3, UBE2I, UBE2J2, UBE3A, UBL7, UBXN11, UBXN7, UGDH, UGGT1, UGP2, UMAD1, AC007161.3, UNC45A, UQCC1, URGCP-MRPS24, URGCP, USMG5, USP16, USP21, USP28, USP3, USP33, USP35, USP54, USP9Y, USPL1, UTP15, VARS2, VASH2, VAV3, VDAC1, VDAC2, VDR, VEZT, VGF, VIL1, VILL, VIPR1, VPS29, VPS37C, VPS8, VPS9D1, VRK2, VWA1, VWA5A, WARS, WASF1, WASHC5, WBP5, WDHD1, WDPCP, WDR37, WDR53, WDR6, WDR72, WDR74, WDR81, WDR86, WDYHV1, WFDC3, WHSC1, WIPF1, WSCD2, WWP2, XAGE1A, XAGE1B, XKR9, XPNPEP1, XRCC3, XRN2, XXYLT1, YIF1A, YIF1B, YIPF1, YIPF5, YPEL5, YWHAB, YWHAZ, YY1AP1, ZBTB1, ZBTB14, ZBTB18, ZBTB20, ZBTB21, ZBTB25, ZBTB33, ZBTB34, ZBTB38, ZBTB43, ZBTB49, ZBTB7B, ZBTB7C, ZBTB8OS, ZC3H11A, ZBED6, ZC3H13, ZCCHC17, ZCCHC7, ZDHHC11, ZDHHC13, ZEB2, ZFAND5, ZFAND6, ZFP1, ZFP62, ZFX, ZFYVE16, ZFYVE19, ZFYVE20, ZFYVE27, ZHX2, AC016405.1, ZHX3, ZIK1, ZIM2, PEG3, ZKSCAN1, ZKSCAN3, ZKSCAN8, ZMAT3, ZMAT5, ZMIZ2, ZMYM6, ZMYND11, ZNF10, AC026786.1, ZNF133, ZNF146, ZNF16, ZNF177, ZNF18, ZNF200, ZNF202, ZNF211, ZNF219, ZNF226, ZNF227, ZNF23, AC010547.4, ZNF23, AC010547.9, ZNF239, ZNF248, ZNF25, ZNF253, ZNF254, ZNF254, AC092279.1, ZNF263, ZNF274, ZNF275, ZNF28, ZNF468, ZNF283, ZNF287, ZNF3, ZNF320, ZNF322, ZNF324B, ZNF331, ZNF334, ZNF34, ZNF350, ZNF385A, ZNF395, FBXO16, ZNF415, ZNF418, ZNF43, ZNF433-AS1, AC008770.4, ZNF438, ZNF444, ZNF445, ZNF467, ZNF480, ZNF493, ZNF493, CTD-2561J22.3, ZNF502, ZNF507, ZNF512, AC074091.1, ZNF512, RP11-158113.2, ZNF512B, ZNF512B, SAMD10, ZNF521, ZNF532, ZNF544, AC020915.5, ZNF544, CTD-3138B18.4, ZNF559, ZNF177, ZNF562, ZNF567, ZNF569, ZNF570, ZNF571-AS1, ZNF540, ZNF577, ZNF580, ZNF581, ZNF580, ZNF581, CCDC106, ZNF600, ZNF611, ZNF613, ZNF615, ZNF619, ZNF620, ZNF639, ZNF652, ZNF665, ZNF667, ZNF668, ZNF671, ZNF682, ZNF687, ZNF691, ZNF696, ZNF701, ZNF706, ZNF707, ZNF714, ZNF717, ZNF718, ZNF720, ZNF721, ZNF730, ZNF763, ZNF780B, AC005614.5, ZNF782, ZNF786, ZNF79, ZNF791, ZNF81, ZNF83, ZNF837, ZNF839, ZNF84, ZNF845, ZNF846, ZNF865, ZNF91, ZNF92, ZNHIT3, ZSCAN21, ZSCAN25, ZSCAN30, and ZSCAN32.

In some embodiments, the gene encoding a target sequence comprises the HTT gene. In some embodiments, the gene encoding a target sequence comprises the MYB gene. In some embodiments, the gene encoding a target sequence comprises the SMN2 gene. In some embodiments, the gene encoding a target sequence comprises the FOXM1 gene.

Exemplary genes that may be modulated by the compounds of Formula (I) or (II) described herein may also include, inter alia, AC005258.1, AC005943.1, AC007849.1, AC008770.2, AC010487.3, AC011477.4, AC012651.1, AC012531.3, AC034102.2, AC073896.4, AC104472.3, AL109811.3, AL133342.1, AL137782.1, AL157871.5, AF241726.2, AL355336.1, AL358113.1, AL360181.3, AL445423.2, AL691482.3, AP001267.5, RF01169, and RF02271.

The compounds described herein may further be used to modulate a sequence comprising a particular splice site sequence, e.g., an RNA sequence (e.g., a pre-mRNA sequence). In some embodiments, the splice site sequence comprises a 5′ splice site sequence. In some embodiments, the splice site sequence comprises a 3′ splice site sequence. Exemplary gene sequences and splice site sequences (e.g., 5′ splice site sequences) include AAAgcaaguu (SEQ ID NO: 1), AAAguaaaaa (SEQ ID NO: 2), AAAguaaaau (SEQ ID NO: 3), AAAguaaagu (SEQ ID NO: 4), AAAguaaaua (SEQ ID NO: 5), AAAguaaaug (SEQ ID NO: 6), AAAguaaauu (SEQ ID NO: 7), AAAguaacac (SEQ ID NO: 8), AAAguaacca (SEQ ID NO: 9), AAAguaacuu (SEQ ID NO: 10), AAAguaagaa (SEQ ID NO: 11), AAAguaagac (SEQ ID NO: 12), AAAguaagag (SEQ ID NO: 13), AAAguaagau (SEQ ID NO: 14), AAAguaagca (SEQ ID NO: 15), AAAguaagcc (SEQ ID NO: 16), AAAguaagcu (SEQ ID NO: 17), AAAguaagga (SEQ ID NO: 18), AAAguaaggg (SEQ ID NO: 19), AAAguaaggu (SEQ ID NO: 20), AAAguaagua (SEQ ID NO: 21), AAAguaaguc (SEQ ID NO: 22), AAAguaagug (SEQ ID NO: 23), AAAguaaguu (SEQ ID NO: 24), AAAguaaucu (SEQ ID NO: 25), AAAguaauua (SEQ ID NO: 26), AAAguacaaa (SEQ ID NO: 27), AAAguaccgg (SEQ ID NO: 28), AAAguacuag (SEQ ID NO: 29), AAAguacugg (SEQ ID NO: 30), AAAguacuuc (SEQ ID NO: 31), AAAguacuug (SEQ ID NO: 32), AAAguagcuu (SEQ ID NO: 33), AAAguaggag (SEQ ID NO: 34), AAAguaggau (SEQ ID NO: 35), AAAguagggg (SEQ ID NO: 36), AAAguaggua (SEQ ID NO: 37), AAAguaguaa (SEQ ID NO: 38), AAAguauauu (SEQ ID NO: 39), AAAguauccu (SEQ ID NO: 40), AAAguaucuc (SEQ ID NO: 41), AAAguaugga (SEQ ID NO: 42), AAAguaugua (SEQ ID NO: 43), AAAguaugug (SEQ ID NO: 44), AAAguauguu (SEQ ID NO: 45), AAAguauugg (SEQ ID NO: 46), AAAguauuuu (SEQ ID NO: 47), AAAgucagau (SEQ ID NO: 48), AAAgucugag (SEQ ID NO: 49), AAAgugaaua (SEQ ID NO: 50), AAAgugagaa (SEQ ID NO: 51), AAAgugagac (SEQ ID NO: 52), AAAgugagag (SEQ ID NO: 53), AAAgugagau (SEQ ID NO: 54), AAAgugagca (SEQ ID NO: 55), AAAgugagcu (SEQ ID NO: 56), AAAgugaggg (SEQ ID NO: 57), AAAgugagua (SEQ ID NO: 58), AAAgugaguc (SEQ ID NO: 59), AAAgugagug (SEQ ID NO: 60), AAAgugaguu (SEQ ID NO: 61), AAAgugcguc (SEQ ID NO: 62), AAAgugcuga (SEQ ID NO: 63), AAAguggguc (SEQ ID NO: 64), AAAguggguu (SEQ ID NO: 65), AAAgugguaa (SEQ ID NO: 66), AAAguguaug (SEQ ID NO: 67), AAAgugugug (SEQ ID NO: 68), AAAguguguu (SEQ ID NO: 69), AAAguuaagu (SEQ ID NO: 70), AAAguuacuu (SEQ ID NO: 71), AAAguuagug (SEQ ID NO: 72), AAAguuaugu (SEQ ID NO: 73), AAAguugagu (SEQ ID NO: 74), AAAguuugua (SEQ ID NO: 75), AACguaaaac (SEQ ID NO: 76), AACguaaagc (SEQ ID NO: 77), AACguaaagg (SEQ ID NO: 78), AACguaagca (SEQ ID NO: 79), AACguaaggg (SEQ ID NO: 80), AACguaaguc (SEQ ID NO: 81), AACguaagug (SEQ ID NO: 82), AACguaaugg (SEQ ID NO: 83), AACguaguga (SEQ ID NO: 84), AACguaugua (SEQ ID NO: 85), AACguauguu (SEQ ID NO: 86), AACgugagca (SEQ ID NO: 87), AACgugagga (SEQ ID NO: 88), AACgugauuu (SEQ ID NO: 89), AACgugggau (SEQ ID NO: 90), AACgugggua (SEQ ID NO: 91), AACguguguu (SEQ ID NO: 92), AACguuggua (SEQ ID NO: 93), AAGgcaaauu (SEQ ID NO: 94), AAGgcaagag (SEQ ID NO: 95), AAGgcaagau (SEQ ID NO: 96), AAGgcaagcc (SEQ ID NO: 97), AAGgcaagga (SEQ ID NO: 98), AAGgcaaggg (SEQ ID NO: 99), AAGgcaagug (SEQ ID NO: 100), AAGgcaaguu (SEQ ID NO: 101), AAGgcacugc (SEQ ID NO: 102), AAGgcagaaa (SEQ ID NO: 103), AAGgcaggau (SEQ ID NO: 104), AAGgcaggca (SEQ ID NO: 105), AAGgcaggga (SEQ ID NO: 106), AAGgcagggg (SEQ ID NO: 107), AAGgcaggua (SEQ ID NO: 108), AAGgcaggug (SEQ ID NO: 109), AAGgcaucuc (SEQ ID NO: 110), AAGgcaugcu (SEQ ID NO: 111), AAGgcaugga (SEQ ID NO: 112), AAGgcauguu (SEQ ID NO: 113), AAGgcauuau (SEQ ID NO: 114), AAGgcgagcu (SEQ ID NO: 115), AAGgcgaguc (SEQ ID NO: 116), AAGgegaguu (SEQ ID NO: 117), AAGgcuagcc (SEQ ID NO: 118), AAGguaaaaa (SEQ ID NO: 119), AAGguaaaac (SEQ ID NO: 120), AAGguaaaag (SEQ ID NO: 121), AAGguaaaau (SEQ ID NO: 122), AAGguaaaca (SEQ ID NO: 123), AAGguaaacc (SEQ ID NO: 124), AAGguaaacu (SEQ ID NO: 125), AAGguaaaga (SEQ ID NO: 126), AAGguaaagc (SEQ ID NO: 127), AAGguaaagg (SEQ ID NO: 128), AAGguaaagu (SEQ ID NO: 129), AAGguaaaua (SEQ ID NO: 130), AAGguaaauc (SEQ ID NO: 131), AAGguaaaug (SEQ ID NO: 132), AAGguaaauu (SEQ ID NO: 133), AAGguaacaa (SEQ ID NO: 134), AAGguaacau (SEQ ID NO: 135), AAGguaaccc (SEQ ID NO: 136), AAGguaacua (SEQ ID NO: 137), AAGguaacuc (SEQ ID NO: 138), AAGguaacug (SEQ ID NO: 139), AAGguaacuu (SEQ ID NO: 140), AAGguaagaa (SEQ ID NO: 141), AAGguaagac (SEQ ID NO: 142), AAGguaagag (SEQ ID NO: 143), AAGguaagau (SEQ ID NO: 144), AAGguaagca (SEQ ID NO: 145), AAGguaagcc (SEQ ID NO: 146), AAGguaagcg (SEQ ID NO: 147), AAGguaagcu (SEQ ID NO: 148), AAGguaagga (SEQ ID NO: 149), AAGguaaggc (SEQ ID NO: 150), AAGguaaggg (SEQ ID NO: 151), AAGguaaggu (SEQ ID NO: 152), AAGguaagua (SEQ ID NO: 153), AAGguaaguc (SEQ ID NO: 154), AAGguaagug (SEQ ID NO: 155), AAGguaaguu (SEQ ID NO: 156), AAGguaauaa (SEQ ID NO: 157), AAGguaauac (SEQ ID NO: 158), AAGguaauag (SEQ ID NO: 159), AAGguaauau (SEQ ID NO: 160), AAGguaauca (SEQ ID NO: 161), AAGguaaucc (SEQ ID NO: 162), AAGguaaucu (SEQ ID NO: 163), AAGguaauga (SEQ ID NO: 164), AAGguaaugc (SEQ ID NO: 165), AAGguaaugg (SEQ ID NO: 166), AAGguaaugu (SEQ ID NO: 167), AAGguaauua (SEQ ID NO: 168), AAGguaauuc (SEQ ID NO: 169), AAGguaauug (SEQ ID NO: 170), AAGguaauuu (SEQ ID NO: 171), AAGguacaaa (SEQ ID NO: 172), AAGguacaag (SEQ ID NO: 173), AAGguacaau (SEQ ID NO: 174), AAGguacacc (SEQ ID NO: 175), AAGguacacu (SEQ ID NO: 176), AAGguacagg (SEQ ID NO: 177), AAGguacagu (SEQ ID NO: 178), AAGguacaua (SEQ ID NO: 179), AAGguacaug (SEQ ID NO: 180), AAGguacauu (SEQ ID NO: 181), AAGguaccaa (SEQ ID NO: 182), AAGguaccag (SEQ ID NO: 183), AAGguaccca (SEQ ID NO: 184), AAGguacccu (SEQ ID NO: 185), AAGguaccuc (SEQ ID NO: 186), AAGguaccug (SEQ ID NO: 187), AAGguaccuu (SEQ ID NO: 188), AAGguacgaa (SEQ ID NO: 189), AAGguacggg (SEQ ID NO: 190), AAGguacggu (SEQ ID NO: 191), AAGguacguc (SEQ ID NO: 192), AAGguacguu (SEQ ID NO: 193), AAGguacuaa (SEQ ID NO: 194), AAGguacuau (SEQ ID NO: 195), AAGguacucu (SEQ ID NO: 196), AAGguacuga (SEQ ID NO: 197), AAGguacugc (SEQ ID NO: 198), AAGguacugu (SEQ ID NO: 199), AAGguacuuc (SEQ ID NO: 200), AAGguacuug (SEQ ID NO: 201), AAGguacuuu (SEQ ID NO: 202), AAGguagaaa (SEQ ID NO: 203), AAGguagaac (SEQ ID NO: 204), AAGguagaca (SEQ ID NO: 205), AAGguagacc (SEQ ID NO: 206), AAGguagacu (SEQ ID NO: 207), AAGguagagu (SEQ ID NO: 208), AAGguagaua (SEQ ID NO: 209), AAGguagcaa (SEQ ID NO: 210), AAGguagcag (SEQ ID NO: 211), AAGguagcca (SEQ ID NO: 212), AAGguagccu (SEQ ID NO: 213), AAGguagcua (SEQ ID NO: 214), AAGguagcug (SEQ ID NO: 215), AAGguagcuu (SEQ ID NO: 216), AAGguaggaa (SEQ ID NO: 217), AAGguaggag (SEQ ID NO: 218), AAGguaggau (SEQ ID NO: 219), AAGguaggca (SEQ ID NO: 220), AAGguaggcc (SEQ ID NO: 221), AAGguaggcu (SEQ ID NO: 222), AAGguaggga (SEQ ID NO: 223), AAGguagggc (SEQ ID NO: 224), AAGguagggg (SEQ ID NO: 225), AAGguagggu (SEQ ID NO: 226), AAGguaggua (SEQ ID NO: 227), AAGguagguc (SEQ ID NO: 228), AAGguaggug (SEQ ID NO: 229), AAGguagguu (SEQ ID NO: 230), AAGguaguaa (SEQ ID NO: 231), AAGguaguag (SEQ ID NO: 232), AAGguagucu (SEQ ID NO: 233), AAGguagugc (SEQ ID NO: 234), AAGguagugg (SEQ ID NO: 235), AAGguaguuc (SEQ ID NO: 236), AAGguaguuu (SEQ ID NO: 237), AAGguauaaa (SEQ ID NO: 238), AAGguauaau (SEQ ID NO: 239), AAGguauaca (SEQ ID NO: 240), AAGguauacu (SEQ ID NO: 241), AAGguauaua (SEQ ID NO: 242), AAGguauauc (SEQ ID NO: 243), AAGguauaug (SEQ ID NO: 244), AAGguauauu (SEQ ID NO: 245), AAGguaucac (SEQ ID NO: 246), AAGguaucag (SEQ ID NO: 247), AAGguauccc (SEQ ID NO: 248), AAGguauccu (SEQ ID NO: 249), AAGguaucuc (SEQ ID NO: 250), AAGguaucug (SEQ ID NO: 251), AAGguaucuu (SEQ ID NO: 252), AAGguaugaa (SEQ ID NO: 253), AAGguaugac (SEQ ID NO: 254), AAGguaugag (SEQ ID NO: 255), AAGguaugau (SEQ ID NO: 256), AAGguaugca (SEQ ID NO: 257), AAGguaugcc (SEQ ID NO: 258), AAGguaugcu (SEQ ID NO: 259), AAGguaugga (SEQ ID NO: 260), AAGguauggc (SEQ ID NO: 261), AAGguauggg (SEQ ID NO: 262), AAGguaugua (SEQ ID NO: 263), AAGguauguc (SEQ ID NO: 264), AAGguaugug (SEQ ID NO: 265), AAGguauguu (SEQ ID NO: 266), AAGguauuaa (SEQ ID NO: 267), AAGguauuac (SEQ ID NO: 268), AAGguauuag (SEQ ID NO: 269), AAGguauuau (SEQ ID NO: 270), AAGguauucc (SEQ ID NO: 271), AAGguauuga (SEQ ID NO: 272), AAGguauugu (SEQ ID NO: 273), AAGguauuua (SEQ ID NO: 274), AAGguauuuc (SEQ ID NO: 275), AAGguauuug (SEQ ID NO: 276), AAGguauuuu (SEQ ID NO: 277), AAGgucaaau (SEQ ID NO: 278), AAGgucaaga (SEQ ID NO: 279), AAGgucaagu (SEQ ID NO: 280), AAGgucacag (SEQ ID NO: 281), AAGgucagaa (SEQ ID NO: 282), AAGgucagac (SEQ ID NO: 283), AAGgucagag (SEQ ID NO: 284), AAGgucagca (SEQ ID NO: 285), AAGgucagcc (SEQ ID NO: 286), AAGgucageg (SEQ ID NO: 287), AAGgucagcu (SEQ ID NO: 288), AAGgucagga (SEQ ID NO: 289), AAGgucaggc (SEQ ID NO: 290), AAGgucaggg (SEQ ID NO: 291), AAGgucaggu (SEQ ID NO: 292), AAGgucagua (SEQ ID NO: 293), AAGgucaguc (SEQ ID NO: 294), AAGgucagug (SEQ ID NO: 295), AAGgucaguu (SEQ ID NO: 296), AAGgucauag (SEQ ID NO: 297), AAGgucaucu (SEQ ID NO: 298), AAGguccaca (SEQ ID NO: 299), AAGguccaga (SEQ ID NO: 300), AAGguccaua (SEQ ID NO: 301), AAGgucccag (SEQ ID NO: 302), AAGgucccuc (SEQ ID NO: 303), AAGguccuuc (SEQ ID NO: 304), AAGgucgagg (SEQ ID NO: 305), AAGgucuaau (SEQ ID NO: 306), AAGgucuacc (SEQ ID NO: 307), AAGgucuaua (SEQ ID NO: 308), AAGgucuccu (SEQ ID NO: 309), AAGgucucug (SEQ ID NO: 310), AAGgucucuu (SEQ ID NO: 311), AAGgucugaa (SEQ ID NO: 312), AAGgucugag (SEQ ID NO: 313), AAGgucugga (SEQ ID NO: 314), AAGgucuggg (SEQ ID NO: 315), AAGgucugua (SEQ ID NO: 316), AAGgucuguu (SEQ ID NO: 317), AAGgucuucu (SEQ ID NO: 318), AAGgucuuuu (SEQ ID NO: 319), AAGgugaaac (SEQ ID NO: 320), AAGgugaaag (SEQ ID NO: 321), AAGgugaaau (SEQ ID NO: 322), AAGgugaacu (SEQ ID NO: 323), AAGgugaagc (SEQ ID NO: 324), AAGgugaagg (SEQ ID NO: 325), AAGgugaagu (SEQ ID NO: 326), AAGgugaaua (SEQ ID NO: 327), AAGgugaaug (SEQ ID NO: 328), AAGgugaauu (SEQ ID NO: 329), AAGgugacaa (SEQ ID NO: 330), AAGgugacag (SEQ ID NO: 331), AAGgugacau (SEQ ID NO: 332), AAGgugacug (SEQ ID NO: 333), AAGgugacuu (SEQ ID NO: 334), AAGgugagaa (SEQ ID NO: 335), AAGgugagac (SEQ ID NO: 336), AAGgugagag (SEQ ID NO: 337), AAGgugagau (SEQ ID NO: 338), AAGgugagca (SEQ ID NO: 339), AAGgugagcc (SEQ ID NO: 340), AAGgugageg (SEQ ID NO: 341), AAGgugagcu (SEQ ID NO: 342), AAGgugagga (SEQ ID NO: 343), AAGgugaggc (SEQ ID NO: 344), AAGgugaggg (SEQ ID NO: 345), AAGgugaggu (SEQ ID NO: 346), AAGgugagua (SEQ ID NO: 347), AAGgugaguc (SEQ ID NO: 348), AAGgugagug (SEQ ID NO: 349), AAGgugaguu (SEQ ID NO: 350), AAGgugauaa (SEQ ID NO: 351), AAGgugauca (SEQ ID NO: 352), AAGgugaucc (SEQ ID NO: 353), AAGgugauga (SEQ ID NO: 354), AAGgugaugc (SEQ ID NO: 355), AAGgugaugu (SEQ ID NO: 356), AAGgugauua (SEQ ID NO: 357), AAGgugauug (SEQ ID NO: 358), AAGgugauuu (SEQ ID NO: 359), AAGgugcaca (SEQ ID NO: 360), AAGgugcauc (SEQ ID NO: 361), AAGgugcccu (SEQ ID NO: 362), AAGgugccug (SEQ ID NO: 363), AAGgugcgug (SEQ ID NO: 364), AAGgugeguu (SEQ ID NO: 365), AAGgugcucc (SEQ ID NO: 366), AAGgugcuga (SEQ ID NO: 367), AAGgugcugc (SEQ ID NO: 368), AAGgugcugg (SEQ ID NO: 369), AAGgugcuua (SEQ ID NO: 370), AAGgugcuuu (SEQ ID NO: 371), AAGguggaua (SEQ ID NO: 372), AAGguggcua (SEQ ID NO: 373), AAGguggcug (SEQ ID NO: 374), AAGguggcuu (SEQ ID NO: 375), AAGgugggaa (SEQ ID NO: 376), AAGgugggag (SEQ ID NO: 377), AAGgugggau (SEQ ID NO: 378), AAGgugggca (SEQ ID NO: 379), AAGgugggcc (SEQ ID NO: 380), AAGgugggcg (SEQ ID NO: 381), AAGgugggga (SEQ ID NO: 382), AAGguggggu (SEQ ID NO: 383), AAGgugggua (SEQ ID NO: 384), AAGgugggug (SEQ ID NO: 385), AAGguggguu (SEQ ID NO: 386), AAGgugguaa (SEQ ID NO: 387), AAGgugguac (SEQ ID NO: 388), AAGgugguau (SEQ ID NO: 389), AAGguggugg (SEQ ID NO: 390), AAGgugguua (SEQ ID NO: 391), AAGgugguuc (SEQ ID NO: 392), AAGgugguuu (SEQ ID NO: 393), AAGguguaag (SEQ ID NO: 394), AAGgugucaa (SEQ ID NO: 395), AAGgugucag (SEQ ID NO: 396), AAGgugucug (SEQ ID NO: 397), AAGgugugaa (SEQ ID NO: 398), AAGgugugag (SEQ ID NO: 399), AAGgugugca (SEQ ID NO: 400), AAGgugugga (SEQ ID NO: 401), AAGguguggu (SEQ ID NO: 402), AAGgugugua (SEQ ID NO: 403), AAGguguguc (SEQ ID NO: 404), AAGgugugug (SEQ ID NO: 405), AAGguguguu (SEQ ID NO: 406), AAGguguucu (SEQ ID NO: 407), AAGguguugc (SEQ ID NO: 408), AAGguguugg (SEQ ID NO: 409), AAGguguuug (SEQ ID NO: 410), AAGguuaaaa (SEQ ID NO: 411), AAGguuaaca (SEQ ID NO: 412), AAGguuaagc (SEQ ID NO: 413), AAGguuaauu (SEQ ID NO: 414), AAGguuacau (SEQ ID NO: 415), AAGguuagaa (SEQ ID NO: 416), AAGguuagau (SEQ ID NO: 417), AAGguuagca (SEQ ID NO: 418), AAGguuagcc (SEQ ID NO: 419), AAGguuagga (SEQ ID NO: 420), AAGguuaggc (SEQ ID NO: 421), AAGguuagua (SEQ ID NO: 422), AAGguuaguc (SEQ ID NO: 423), AAGguuagug (SEQ ID NO: 424), AAGguuaguu (SEQ ID NO: 425), AAGguuauag (SEQ ID NO: 426), AAGguuauga (SEQ ID NO: 427), AAGguucaaa (SEQ ID NO: 428), AAGguucaag (SEQ ID NO: 429), AAGguuccuu (SEQ ID NO: 430), AAGguucggc (SEQ ID NO: 431), AAGguucguu (SEQ ID NO: 432), AAGguucuaa (SEQ ID NO: 433), AAGguucuga (SEQ ID NO: 434), AAGguucuua (SEQ ID NO: 435), AAGguugaau (SEQ ID NO: 436), AAGguugacu (SEQ ID NO: 437), AAGguugagg (SEQ ID NO: 438), AAGguugagu (SEQ ID NO: 439), AAGguugaua (SEQ ID NO: 440), AAGguugcac (SEQ ID NO: 441), AAGguugcug (SEQ ID NO: 442), AAGguuggaa (SEQ ID NO: 443), AAGguuggca (SEQ ID NO: 444), AAGguuggga (SEQ ID NO: 445), AAGguugggg (SEQ ID NO: 446), AAGguuggua (SEQ ID NO: 447), AAGguugguc (SEQ ID NO: 448), AAGguuggug (SEQ ID NO: 449), AAGguugguu (SEQ ID NO: 450), AAGguuguaa (SEQ ID NO: 451), AAGguugucc (SEQ ID NO: 452), AAGguugugc (SEQ ID NO: 453), AAGguuguua (SEQ ID NO: 454), AAGguuuacc (SEQ ID NO: 455), AAGguuuaua (SEQ ID NO: 456), AAGguuuuu (SEQ ID NO: 457), AAGguuuccu (SEQ ID NO: 458), AAGguuucgu (SEQ ID NO: 459), AAGguuugag (SEQ ID NO: 460), AAGguuugca (SEQ ID NO: 461), AAGguuugcc (SEQ ID NO: 462), AAGguuugcu (SEQ ID NO: 463), AAGguuugga (SEQ ID NO: 464), AAGguuuggu (SEQ ID NO: 465), AAGguuugua (SEQ ID NO: 466), AAGguuuguc (SEQ ID NO: 467), AAGguuugug (SEQ ID NO: 468), AAGguuuuaa (SEQ ID NO: 469), AAGguuuuca (SEQ ID NO: 470), AAGguuuucg (SEQ ID NO: 471), AAGguuuugc (SEQ ID NO: 472), AAGguuuugu (SEQ ID NO: 473), AAGguuuuuu (SEQ ID NO: 474), AAUgcaagua (SEQ ID NO: 475), AAUgcaaguc (SEQ ID NO: 476), AAUguaaaca (SEQ ID NO: 477), AAUguaaaua (SEQ ID NO: 478), AAUguaaauc (SEQ ID NO: 479), AAUguaaaug (SEQ ID NO: 480), AAUguaaauu (SEQ ID NO: 481), AAUguaacua (SEQ ID NO: 482), AAUguaagaa (SEQ ID NO: 483), AAUguaagag (SEQ ID NO: 484), AAUguaagau (SEQ ID NO: 485), AAUguaagcc (SEQ ID NO: 486), AAUguaagcu (SEQ ID NO: 487), AAUguaagga (SEQ ID NO: 488), AAUguaagua (SEQ ID NO: 489), AAUguaaguc (SEQ ID NO: 490), AAUguaagug (SEQ ID NO: 491), AAUguaaguu (SEQ ID NO: 492), AAUguaauca (SEQ ID NO: 493), AAUguaauga (SEQ ID NO: 494), AAUguaaugu (SEQ ID NO: 495), AAUguacauc (SEQ ID NO: 496), AAUguacaug (SEQ ID NO: 497), AAUguacgau (SEQ ID NO: 498), AAUguacgua (SEQ ID NO: 499), AAUguacguc (SEQ ID NO: 500), AAUguacgug (SEQ ID NO: 501), AAUguacucu (SEQ ID NO: 502), AAUguaggca (SEQ ID NO: 503), AAUguagguu (SEQ ID NO: 504), AAUguaucua (SEQ ID NO: 505), AAUguaugaa (SEQ ID NO: 506), AAUguaugua (SEQ ID NO: 507), AAUguaugug (SEQ ID NO: 508), AAUguauguu (SEQ ID NO: 509), AAUgucagag (SEQ ID NO: 510), AAUgucagau (SEQ ID NO: 511), AAUgucagcu (SEQ ID NO: 512), AAUgucagua (SEQ ID NO: 513), AAUgucaguc (SEQ ID NO: 514), AAUgucagug (SEQ ID NO: 515), AAUgucaguu (SEQ ID NO: 516), AAUgucggua (SEQ ID NO: 517), AAUgucuguu (SEQ ID NO: 518), AAUgugagaa (SEQ ID NO: 519), AAUgugagca (SEQ ID NO: 520), AAUgugagcc (SEQ ID NO: 521), AAUgugagga (SEQ ID NO: 522), AAUgugagua (SEQ ID NO: 523), AAUgugaguc (SEQ ID NO: 524), AAUgugagug (SEQ ID NO: 525), AAUgugaguu (SEQ ID NO: 526), AAUgugauau (SEQ ID NO: 527), AAUgugcaua (SEQ ID NO: 528), AAUgugcgua (SEQ ID NO: 529), AAUgugcguc (SEQ ID NO: 530), AAUgugggac (SEQ ID NO: 531), AAUguggguc (SEQ ID NO: 532), AAUgugggug (SEQ ID NO: 533), AAUgugguuu (SEQ ID NO: 534), AAUgugugua (SEQ ID NO: 535), AAUguuaagu (SEQ ID NO: 536), AAUguuagaa (SEQ ID NO: 537), AAUguuagau (SEQ ID NO: 538), AAUguuagua (SEQ ID NO: 539), AAUguuggug (SEQ ID NO: 540), ACAgcaagua (SEQ ID NO: 541), ACAguaaaua (SEQ ID NO: 542), ACAguaaaug (SEQ ID NO: 543), ACAguaagaa (SEQ ID NO: 544), ACAguaagca (SEQ ID NO: 545), ACAguaagua (SEQ ID NO: 546), ACAguaaguc (SEQ ID NO: 547), ACAguaagug (SEQ ID NO: 548), ACAguaaguu (SEQ ID NO: 549), ACAguacgua (SEQ ID NO: 550), ACAguaggug (SEQ ID NO: 551), ACAguauaac (SEQ ID NO: 552), ACAguaugua (SEQ ID NO: 553), ACAgucaguu (SEQ ID NO: 554), ACAgugagaa (SEQ ID NO: 555), ACAgugagcc (SEQ ID NO: 556), ACAgugagcu (SEQ ID NO: 557), ACAgugagga (SEQ ID NO: 558), ACAgugaggu (SEQ ID NO: 559), ACAgugagua (SEQ ID NO: 560), ACAgugaguc (SEQ ID NO: 561), ACAgugagug (SEQ ID NO: 562), ACAgugaguu (SEQ ID NO: 563), ACAgugggua (SEQ ID NO: 564), ACAguggguu (SEQ ID NO: 565), ACAguguaaa (SEQ ID NO: 566), ACAguuaagc (SEQ ID NO: 567), ACAguuaagu (SEQ ID NO: 568), ACAguuaugu (SEQ ID NO: 569), ACAguugagu (SEQ ID NO: 570), ACAguuguga (SEQ ID NO: 571), ACCguaagua (SEQ ID NO: 572), ACCgugagaa (SEQ ID NO: 573), ACCgugagca (SEQ ID NO: 574), ACCgugaguu (SEQ ID NO: 575), ACCgugggug (SEQ ID NO: 576), ACGguaaaac (SEQ ID NO: 577), ACGguaacua (SEQ ID NO: 578), ACGguaagua (SEQ ID NO: 579), ACGguaagug (SEQ ID NO: 580), ACGguaaguu (SEQ ID NO: 581), ACGguaauua (SEQ ID NO: 582), ACGguaauuu (SEQ ID NO: 583), ACGguacaau (SEQ ID NO: 584), ACGguacagu (SEQ ID NO: 585), ACGguaccag (SEQ ID NO: 586), ACGguacggu (SEQ ID NO: 587), ACGguacgua (SEQ ID NO: 588), ACGguaggaa (SEQ ID NO: 589), ACGguaggag (SEQ ID NO: 590), ACGguaggug (SEQ ID NO: 591), ACGguaguaa (SEQ ID NO: 592), ACGguauaau (SEQ ID NO: 593), ACGguaugac (SEQ ID NO: 594), ACGguaugcg (SEQ ID NO: 595), ACGguaugua (SEQ ID NO: 596), ACGguauguc (SEQ ID NO: 597), ACGgugaaac (SEQ ID NO: 598), ACGgugaagu (SEQ ID NO: 599), ACGgugaauc (SEQ ID NO: 600), ACGgugacag (SEQ ID NO: 601), ACGgugacca (SEQ ID NO: 602), ACGgugagaa (SEQ ID NO: 603), ACGgugagau (SEQ ID NO: 604), ACGgugagcc (SEQ ID NO: 605), ACGgugagua (SEQ ID NO: 606), ACGgugagug (SEQ ID NO: 607), ACGgugaguu (SEQ ID NO: 608), ACGgugcgug (SEQ ID NO: 609), ACGguggcac (SEQ ID NO: 610), ACGguggggc (SEQ ID NO: 611), ACGgugggug (SEQ ID NO: 612), ACGguguagu (SEQ ID NO: 613), ACGgugucac (SEQ ID NO: 614), ACGgugugua (SEQ ID NO: 615), ACGguguguu (SEQ ID NO: 616), ACGguuagug (SEQ ID NO: 617), ACGguuaguu (SEQ ID NO: 618), ACGguucaau (SEQ ID NO: 619), ACUguaaaua (SEQ ID NO: 620), ACUguaagaa (SEQ ID NO: 621), ACUguaagac (SEQ ID NO: 622), ACUguaagca (SEQ ID NO: 623), ACUguaagcu (SEQ ID NO: 624), ACUguaagua (SEQ ID NO: 625), ACUguaaguc (SEQ ID NO: 626), ACUguaaguu (SEQ ID NO: 627), ACUguacguu (SEQ ID NO: 628), ACUguacugc (SEQ ID NO: 629), ACUguaggcu (SEQ ID NO: 630), ACUguaggua (SEQ ID NO: 631), ACUguauauu (SEQ ID NO: 632), ACUguaugaa (SEQ ID NO: 633), ACUguaugcu (SEQ ID NO: 634), ACUguaugug (SEQ ID NO: 635), ACUguauucc (SEQ ID NO: 636), ACUgucagcu (SEQ ID NO: 637), ACUgucagug (SEQ ID NO: 638), ACUgugaacg (SEQ ID NO: 639), ACUgugagca (SEQ ID NO: 640), ACUgugageg (SEQ ID NO: 641), ACUgugagcu (SEQ ID NO: 642), ACUgugagua (SEQ ID NO: 643), ACUgugaguc (SEQ ID NO: 644), ACUgugagug (SEQ ID NO: 645), ACUgugaguu (SEQ ID NO: 646), ACUgugggua (SEQ ID NO: 647), ACUgugugug (SEQ ID NO: 648), ACUguuaagu (SEQ ID NO: 649), AGAgcaagua (SEQ ID NO: 650), AGAguaaaac (SEQ ID NO: 651), AGAguaaacg (SEQ ID NO: 652), AGAguaaaga (SEQ ID NO: 653), AGAguaaagu (SEQ ID NO: 654), AGAguaaauc (SEQ ID NO: 655), AGAguaaaug (SEQ ID NO: 656), AGAguaacau (SEQ ID NO: 657), AGAguaacua (SEQ ID NO: 658), AGAguaagaa (SEQ ID NO: 659), AGAguaagac (SEQ ID NO: 660), AGAguaagag (SEQ ID NO: 661), AGAguaagau (SEQ ID NO: 662), AGAguaagca (SEQ ID NO: 663), AGAguaagcu (SEQ ID NO: 664), AGAguaagga (SEQ ID NO: 665), AGAguaaggc (SEQ ID NO: 666), AGAguaaggg (SEQ ID NO: 667), AGAguaaggu (SEQ ID NO: 668), AGAguaaguc (SEQ ID NO: 669), AGAguaagug (SEQ ID NO: 670), AGAguaaguu (SEQ ID NO: 671), AGAguaauaa (SEQ ID NO: 672), AGAguaaugu (SEQ ID NO: 673), AGAguaauuc (SEQ ID NO: 674), AGAguaauuu (SEQ ID NO: 675), AGAguacacc (SEQ ID NO: 676), AGAguaccug (SEQ ID NO: 677), AGAguacgug (SEQ ID NO: 678), AGAguacucu (SEQ ID NO: 679), AGAguacuga (SEQ ID NO: 680), AGAguacuuu (SEQ ID NO: 681), AGAguagcug (SEQ ID NO: 682), AGAguaggaa (SEQ ID NO: 683), AGAguaggga (SEQ ID NO: 684), AGAguagggu (SEQ ID NO: 685), AGAguagguc (SEQ ID NO: 686), AGAguaggug (SEQ ID NO: 687), AGAguagguu (SEQ ID NO: 688), AGAguauaua (SEQ ID NO: 689), AGAguauauu (SEQ ID NO: 690), AGAguaugaa (SEQ ID NO: 691), AGAguaugac (SEQ ID NO: 692), AGAguaugau (SEQ ID NO: 693), AGAguauguc (SEQ ID NO: 694), AGAguaugug (SEQ ID NO: 695), AGAguauguu (SEQ ID NO: 696), AGAguauuaa (SEQ ID NO: 697), AGAguauuau (SEQ ID NO: 698), AGAgucagug (SEQ ID NO: 699), AGAgugagac (SEQ ID NO: 700), AGAgugagag (SEQ ID NO: 701), AGAgugagau (SEQ ID NO: 702), AGAgugagca (SEQ ID NO: 703), AGAgugagua (SEQ ID NO: 704), AGAgugaguc (SEQ ID NO: 705), AGAgugagug (SEQ ID NO: 706), AGAgugaguu (SEQ ID NO: 707), AGAgugcguc (SEQ ID NO: 708), AGAgugggga (SEQ ID NO: 709), AGAgugggug (SEQ ID NO: 710), AGAgugugug (SEQ ID NO: 711), AGAguguuuc (SEQ ID NO: 712), AGAguuagua (SEQ ID NO: 713), AGAguugaga (SEQ ID NO: 714), AGAguugagu (SEQ ID NO: 715), AGAguugguu (SEQ ID NO: 716), AGAguuugau (SEQ ID NO: 717), AGCguaagcu (SEQ ID NO: 718), AGCguaagug (SEQ ID NO: 719), AGCgugagcc (SEQ ID NO: 720), AGCgugagug (SEQ ID NO: 721), AGCguuguuc (SEQ ID NO: 722), AGGgcagagu (SEQ ID NO: 723), AGGgcagccu (SEQ ID NO: 724), AGGgcuagua (SEQ ID NO: 725), AGGguaaaga (SEQ ID NO: 726), AGGguaaaua (SEQ ID NO: 727), AGGguaaauc (SEQ ID NO: 728), AGGguaaauu (SEQ ID NO: 729), AGGguaacca (SEQ ID NO: 730), AGGguaacug (SEQ ID NO: 731), AGGguaacuu (SEQ ID NO: 732), AGGguaagaa (SEQ ID NO: 733), AGGguaagag (SEQ ID NO: 734), AGGguaagau (SEQ ID NO: 735), AGGguaagca (SEQ ID NO: 736), AGGguaagga (SEQ ID NO: 737), AGGguaaggc (SEQ ID NO: 738), AGGguaaggg (SEQ ID NO: 739), AGGguaagua (SEQ ID NO: 740), AGGguaaguc (SEQ ID NO: 741), AGGguaagug (SEQ ID NO: 742), AGGguaaguu (SEQ ID NO: 743), AGGguaauac (SEQ ID NO: 744), AGGguaauga (SEQ ID NO: 745), AGGguaauua (SEQ ID NO: 746), AGGguaauuu (SEQ ID NO: 747), AGGguacacc (SEQ ID NO: 748), AGGguacagu (SEQ ID NO: 749), AGGguacggu (SEQ ID NO: 750), AGGguaggac (SEQ ID NO: 751), AGGguaggag (SEQ ID NO: 752), AGGguaggca (SEQ ID NO: 753), AGGguaggcc (SEQ ID NO: 754), AGGguaggga (SEQ ID NO: 755), AGGguagggu (SEQ ID NO: 756), AGGguagguc (SEQ ID NO: 757), AGGguaggug (SEQ ID NO: 758), AGGguagguu (SEQ ID NO: 759), AGGguauaua (SEQ ID NO: 760), AGGguaugac (SEQ ID NO: 761), AGGguaugag (SEQ ID NO: 762), AGGguaugau (SEQ ID NO: 763), AGGguaugca (SEQ ID NO: 764), AGGguaugcu (SEQ ID NO: 765), AGGguauggg (SEQ ID NO: 766), AGGguauggu (SEQ ID NO: 767), AGGguaugua (SEQ ID NO: 768), AGGguauguc (SEQ ID NO: 769), AGGguaugug (SEQ ID NO: 770), AGGguauuac (SEQ ID NO: 771), AGGguauucu (SEQ ID NO: 772), AGGguauuuc (SEQ ID NO: 773), AGGgucagag (SEQ ID NO: 774), AGGgucagca (SEQ ID NO: 775), AGGgucagga (SEQ ID NO: 776), AGGgucaggg (SEQ ID NO: 777), AGGgucagug (SEQ ID NO: 778), AGGgucaguu (SEQ ID NO: 779), AGGguccccu (SEQ ID NO: 780), AGGgucggga (SEQ ID NO: 781), AGGgucugca (SEQ ID NO: 782), AGGgucuguu (SEQ ID NO: 783), AGGgugaaga (SEQ ID NO: 784), AGGgugacua (SEQ ID NO: 785), AGGgugagaa (SEQ ID NO: 786), AGGgugagac (SEQ ID NO: 787), AGGgugagag (SEQ ID NO: 788), AGGgugagca (SEQ ID NO: 789), AGGgugagcc (SEQ ID NO: 790), AGGgugagcu (SEQ ID NO: 791), AGGgugagga (SEQ ID NO: 792), AGGgugaggg (SEQ ID NO: 793), AGGgugaggu (SEQ ID NO: 794), AGGgugagua (SEQ ID NO: 795), AGGgugaguc (SEQ ID NO: 796), AGGgugagug (SEQ ID NO: 797), AGGgugaguu (SEQ ID NO: 798), AGGgugggga (SEQ ID NO: 799), AGGguggggu (SEQ ID NO: 800), AGGgugggua (SEQ ID NO: 801), AGGgugggug (SEQ ID NO: 802), AGGgugugua (SEQ ID NO: 803), AGGgugugug (SEQ ID NO: 804), AGGguuaaug (SEQ ID NO: 805), AGGguuagaa (SEQ ID NO: 806), AGGguuaguu (SEQ ID NO: 807), AGGguuggug (SEQ ID NO: 808), AGGguuugug (SEQ ID NO: 809), AGGguuuguu (SEQ ID NO: 810), AGUguaaaag (SEQ ID NO: 811), AGUguaaaua (SEQ ID NO: 812), AGUguaaauu (SEQ ID NO: 813), AGUguaagaa (SEQ ID NO: 814), AGUguaagag (SEQ ID NO: 815), AGUguaagau (SEQ ID NO: 816), AGUguaagca (SEQ ID NO: 817), AGUguaagcc (SEQ ID NO: 818), AGUguaagua (SEQ ID NO: 819), AGUguaagug (SEQ ID NO: 820), AGUguaaguu (SEQ ID NO: 821), AGUguaauug (SEQ ID NO: 822), AGUguaggac (SEQ ID NO: 823), AGUguagguc (SEQ ID NO: 824), AGUguaugag (SEQ ID NO: 825), AGUguaugua (SEQ ID NO: 826), AGUguauguu (SEQ ID NO: 827), AGUguauugu (SEQ ID NO: 828), AGUguauuua (SEQ ID NO: 829), AGUgucaguc (SEQ ID NO: 830), AGUgugagag (SEQ ID NO: 831), AGUgugagca (SEQ ID NO: 832), AGUgugagcc (SEQ ID NO: 833), AGUgugagcu (SEQ ID NO: 834), AGUgugagua (SEQ ID NO: 835), AGUgugaguc (SEQ ID NO: 836), AGUgugagug (SEQ ID NO: 837), AGUgugaguu (SEQ ID NO: 838), AGUgugggua (SEQ ID NO: 839), AGUgugggug (SEQ ID NO: 840), AGUgugugua (SEQ ID NO: 841), AGUguuccua (SEQ ID NO: 842), AGUguugggg (SEQ ID NO: 843), AGUguuucag (SEQ ID NO: 844), AUAguaaaua (SEQ ID NO: 845), AUAguaagac (SEQ ID NO: 846), AUAguaagau (SEQ ID NO: 847), AUAguaagca (SEQ ID NO: 848), AUAguaagua (SEQ ID NO: 849), AUAguaagug (SEQ ID NO: 850), AUAguaaguu (SEQ ID NO: 851), AUAguaggua (SEQ ID NO: 852), AUAguauguu (SEQ ID NO: 853), AUAgucucac (SEQ ID NO: 854), AUAgugagac (SEQ ID NO: 855), AUAgugagag (SEQ ID NO: 856), AUAgugagau (SEQ ID NO: 857), AUAgugagcc (SEQ ID NO: 858), AUAgugaggc (SEQ ID NO: 859), AUAgugagua (SEQ ID NO: 860), AUAgugaguc (SEQ ID NO: 861), AUAgugagug (SEQ ID NO: 862), AUAgugcguc (SEQ ID NO: 863), AUAgugugua (SEQ ID NO: 864), AUAguucagu (SEQ ID NO: 865), AUCguaagcc (SEQ ID NO: 866), AUCguaaguu (SEQ ID NO: 867), AUCguauucc (SEQ ID NO: 868), AUCgugagua (SEQ ID NO: 869), AUGgcaagcg (SEQ ID NO: 870), AUGgcaagga (SEQ ID NO: 871), AUGgcaaguu (SEQ ID NO: 872), AUGgcaggua (SEQ ID NO: 873), AUGgcaugug (SEQ ID NO: 874), AUGgegccau (SEQ ID NO: 875), AUGgcuugug (SEQ ID NO: 876), AUGguaaaac (SEQ ID NO: 877), AUGguaaaau (SEQ ID NO: 878), AUGguaaacc (SEQ ID NO: 879), AUGguaaaga (SEQ ID NO: 880), AUGguaaaua (SEQ ID NO: 881), AUGguaaaug (SEQ ID NO: 882), AUGguaaauu (SEQ ID NO: 883), AUGguaacag (SEQ ID NO: 884), AUGguaacau (SEQ ID NO: 885), AUGguaacua (SEQ ID NO: 886), AUGguaacuc (SEQ ID NO: 887), AUGguaacuu (SEQ ID NO: 888), AUGguaagaa (SEQ ID NO: 889), AUGguaagac (SEQ ID NO: 890), AUGguaagag (SEQ ID NO: 891), AUGguaagau (SEQ ID NO: 892), AUGguaagca (SEQ ID NO: 893), AUGguaagcc (SEQ ID NO: 894), AUGguaagcu (SEQ ID NO: 895), AUGguaagga (SEQ ID NO: 896), AUGguaaggg (SEQ ID NO: 897), AUGguaagua (SEQ ID NO: 898), AUGguaaguc (SEQ ID NO: 899), AUGguaagug (SEQ ID NO: 900), AUGguaaguu (SEQ ID NO: 901), AUGguaauaa (SEQ ID NO: 902), AUGguaauau (SEQ ID NO: 903), AUGguaauga (SEQ ID NO: 904), AUGguaaugg (SEQ ID NO: 905), AUGguaauug (SEQ ID NO: 906), AUGguaauuu (SEQ ID NO: 907), AUGguacagc (SEQ ID NO: 908), AUGguacauc (SEQ ID NO: 909), AUGguaccag (SEQ ID NO: 910), AUGguaccug (SEQ ID NO: 911), AUGguacgag (SEQ ID NO: 912), AUGguacggu (SEQ ID NO: 913), AUGguagauc (SEQ ID NO: 914), AUGguagcag (SEQ ID NO: 915), AUGguagcug (SEQ ID NO: 916), AUGguaggaa (SEQ ID NO: 917), AUGguaggau (SEQ ID NO: 918), AUGguaggca (SEQ ID NO: 919), AUGguaggcu (SEQ ID NO: 920), AUGguagggg (SEQ ID NO: 921), AUGguagggu (SEQ ID NO: 922), AUGguaggua (SEQ ID NO: 923), AUGguaggug (SEQ ID NO: 924), AUGguaguuu (SEQ ID NO: 925), AUGguauagu (SEQ ID NO: 926), AUGguauaua (SEQ ID NO: 927), AUGguaucag (SEQ ID NO: 928), AUGguaucuu (SEQ ID NO: 929), AUGguaugau (SEQ ID NO: 930), AUGguaugca (SEQ ID NO: 931), AUGguaugcc (SEQ ID NO: 932), AUGguaugcg (SEQ ID NO: 933), AUGguaugcu (SEQ ID NO: 934), AUGguaugga (SEQ ID NO: 935), AUGguauggc (SEQ ID NO: 936), AUGguaugug (SEQ ID NO: 937), AUGguauguu (SEQ ID NO: 938), AUGguauuau (SEQ ID NO: 939), AUGguauuga (SEQ ID NO: 940), AUGguauuug (SEQ ID NO: 941), AUGgucaggg (SEQ ID NO: 942), AUGgucaguc (SEQ ID NO: 943), AUGgucagug (SEQ ID NO: 944), AUGgucauuu (SEQ ID NO: 945), AUGgugaaaa (SEQ ID NO: 946), AUGgugaaac (SEQ ID NO: 947), AUGgugaaau (SEQ ID NO: 948), AUGgugaacu (SEQ ID NO: 949), AUGgugaaga (SEQ ID NO: 950), AUGgugacgu (SEQ ID NO: 951), AUGgugagaa (SEQ ID NO: 952), AUGgugagac (SEQ ID NO: 953), AUGgugagag (SEQ ID NO: 954), AUGgugagca (SEQ ID NO: 955), AUGgugagcc (SEQ ID NO: 956), AUGgugagcg (SEQ ID NO: 957), AUGgugagcu (SEQ ID NO: 958), AUGgugaggc (SEQ ID NO: 959), AUGgugaggg (SEQ ID NO: 960), AUGgugagua (SEQ ID NO: 961), AUGgugaguc (SEQ ID NO: 962), AUGgugagug (SEQ ID NO: 963), AUGgugaguu (SEQ ID NO: 964), AUGgugauuu (SEQ ID NO: 965), AUGgugegau (SEQ ID NO: 966), AUGgugcgug (SEQ ID NO: 967), AUGgugggua (SEQ ID NO: 968), AUGgugggug (SEQ ID NO: 969), AUGguggguu (SEQ ID NO: 970), AUGgugguua (SEQ ID NO: 971), AUGguguaag (SEQ ID NO: 972), AUGgugugaa (SEQ ID NO: 973), AUGgugugua (SEQ ID NO: 974), AUGgugugug (SEQ ID NO: 975), AUGguuacuc (SEQ ID NO: 976), AUGguuagca (SEQ ID NO: 977), AUGguuaguc (SEQ ID NO: 978), AUGguuagug (SEQ ID NO: 979), AUGguuaguu (SEQ ID NO: 980), AUGguucagu (SEQ ID NO: 981), AUGguucguc (SEQ ID NO: 982), AUGguuggua (SEQ ID NO: 983), AUGguugguc (SEQ ID NO: 984), AUGguugguu (SEQ ID NO: 985), AUGguuguuu (SEQ ID NO: 986), AUGguuugca (SEQ ID NO: 987), AUGguuugua (SEQ ID NO: 988), AUUgcaagua (SEQ ID NO: 989), AUUguaaaua (SEQ ID NO: 990), AUUguaagau (SEQ ID NO: 991), AUUguaagca (SEQ ID NO: 992), AUUguaagga (SEQ ID NO: 993), AUUguaaggc (SEQ ID NO: 994), AUUguaagua (SEQ ID NO: 995), AUUguaaguc (SEQ ID NO: 996), AUUguaaguu (SEQ ID NO: 997), AUUguaauua (SEQ ID NO: 998), AUUguaauuu (SEQ ID NO: 999), AUUguacaaa (SEQ ID NO: 1000), AUUguaccuc (SEQ ID NO: 1001), AUUguacgug (SEQ ID NO: 1002), AUUguacuug (SEQ ID NO: 1003), AUUguaggua (SEQ ID NO: 1004), AUUguaugag (SEQ ID NO: 1005), AUUguaugua (SEQ ID NO: 1006), AUUgucuguu (SEQ ID NO: 1007), AUUgugagcu (SEQ ID NO: 1008), AUUgugagua (SEQ ID NO: 1009), AUUgugaguc (SEQ ID NO: 1010), AUUgugaguu (SEQ ID NO: 1011), AUUgugcgug (SEQ ID NO: 1012), AUUgugggug (SEQ ID NO: 1013), AUUguuagug (SEQ ID NO: 1014), CAAguaaaaa (SEQ ID NO: 1015), CAAguaaaua (SEQ ID NO: 1016), CAAguaaauc (SEQ ID NO: 1017), CAAguaaaug (SEQ ID NO: 1018), CAAguaaccc (SEQ ID NO: 1019), CAAguaacua (SEQ ID NO: 1020), CAAguaacug (SEQ ID NO: 1021), CAAguaagaa (SEQ ID NO: 1022), CAAguaagac (SEQ ID NO: 1023), CAAguaagau (SEQ ID NO: 1024), CAAguaaggu (SEQ ID NO: 1025), CAAguaagua (SEQ ID NO: 1026), CAAguaaguc (SEQ ID NO: 1027), CAAguaagug (SEQ ID NO: 1028), CAAguaaguu (SEQ ID NO: 1029), CAAguaaucc (SEQ ID NO: 1030), CAAguaaucu (SEQ ID NO: 1031), CAAguaauua (SEQ ID NO: 1032), CAAguaauuc (SEQ ID NO: 1033), CAAguaauug (SEQ ID NO: 1034), CAAguaauuu (SEQ ID NO: 1035), CAAguacaca (SEQ ID NO: 1036), CAAguacguu (SEQ ID NO: 1037), CAAguacuuu (SEQ ID NO: 1038), CAAguagcug (SEQ ID NO: 1039), CAAguaggau (SEQ ID NO: 1040), CAAguaggua (SEQ ID NO: 1041), CAAguagguc (SEQ ID NO: 1042), CAAguaggug (SEQ ID NO: 1043), CAAguagguu (SEQ ID NO: 1044), CAAguaguuu (SEQ ID NO: 1045), CAAguauaac (SEQ ID NO: 1046), CAAguauaug (SEQ ID NO: 1047), CAAguaucuu (SEQ ID NO: 1048), CAAguaugag (SEQ ID NO: 1049), CAAguaugua (SEQ ID NO: 1050), CAAguauguc (SEQ ID NO: 1051), CAAguaugug (SEQ ID NO: 1052), CAAguauguu (SEQ ID NO: 1053), CAAguauuga (SEQ ID NO: 1054), CAAguauuuc (SEQ ID NO: 1055), CAAgucagac (SEQ ID NO: 1056), CAAgucagua (SEQ ID NO: 1057), CAAgucuaua (SEQ ID NO: 1058), CAAgucugau (SEQ ID NO: 1059), CAAgugacuu (SEQ ID NO: 1060), CAAgugagaa (SEQ ID NO: 1061), CAAgugagac (SEQ ID NO: 1062), CAAgugagca (SEQ ID NO: 1063), CAAgugaggc (SEQ ID NO: 1064), CAAgugaggg (SEQ ID NO: 1065), CAAgugagua (SEQ ID NO: 1066), CAAgugaguc (SEQ ID NO: 1067), CAAgugagug (SEQ ID NO: 1068), CAAgugaucc (SEQ ID NO: 1069), CAAgugaucu (SEQ ID NO: 1070), CAAgugauuc (SEQ ID NO: 1071), CAAgugauug (SEQ ID NO: 1072), CAAgugauuu (SEQ ID NO: 1073), CAAgugccuu (SEQ ID NO: 1074), CAAgugggua (SEQ ID NO: 1075), CAAguggguc (SEQ ID NO: 1076), CAAgugggug (SEQ ID NO: 1077), CAAgugugag (SEQ ID NO: 1078), CAAguuaaaa (SEQ ID NO: 1079), CAAguuaagu (SEQ ID NO: 1080), CAAguaauc (SEQ ID NO: 1081), CAAguuagaa (SEQ ID NO: 1082), CAAguuaguu (SEQ ID NO: 1083), CAAguucaag (SEQ ID NO: 1084), CAAguuccgu (SEQ ID NO: 1085), CAAguuggua (SEQ ID NO: 1086), CAAguuuagu (SEQ ID NO: 1087), CAAguuucca (SEQ ID NO: 1088), CAAguuuguu (SEQ ID NO: 1089), CACguaagag (SEQ ID NO: 1090), CACguaagca (SEQ ID NO: 1091), CACguaauug (SEQ ID NO: 1092), CACguaggac (SEQ ID NO: 1093), CACguaucga (SEQ ID NO: 1094), CACgucaguu (SEQ ID NO: 1095), CACgugagcu (SEQ ID NO: 1096), CACgugaguc (SEQ ID NO: 1097), CACgugagug (SEQ ID NO: 1098), CAGgcaagaa (SEQ ID NO: 1099), CAGgcaagac (SEQ ID NO: 1100), CAGgcaagag (SEQ ID NO: 1101), CAGgcaagga (SEQ ID NO: 1102), CAGgcaagua (SEQ ID NO: 1103), CAGgcaagug (SEQ ID NO: 1104), CAGgcaaguu (SEQ ID NO: 1105), CAGgcacgca (SEQ ID NO: 1106), CAGgcagagg (SEQ ID NO: 1107), CAGgcaggug (SEQ ID NO: 1108), CAGgcaucau (SEQ ID NO: 1109), CAGgcaugaa (SEQ ID NO: 1110), CAGgcaugag (SEQ ID NO: 1111), CAGgcaugca (SEQ ID NO: 1112), CAGgcaugcg (SEQ ID NO: 1113), CAGgcaugug (SEQ ID NO: 1114), CAGgcgagag (SEQ ID NO: 1115), CAGgegccug (SEQ ID NO: 1116), CAGgcgugug (SEQ ID NO: 1117), CAGguaaaaa (SEQ ID NO: 1118), CAGguaaaag (SEQ ID NO: 1119), CAGguaaaca (SEQ ID NO: 1120), CAGguaaacc (SEQ ID NO: 1121), CAGguaaaga (SEQ ID NO: 1122), CAGguaaagc (SEQ ID NO: 1123), CAGguaagu (SEQ ID NO: 1124), CAGguaaaua (SEQ ID NO: 1125), CAGguaaauc (SEQ ID NO: 1126), CAGguaaaug (SEQ ID NO: 1127), CAGguaaauu (SEQ ID NO: 1128), CAGguaacag (SEQ ID NO: 1129), CAGguaacau (SEQ ID NO: 1130), CAGguaacca (SEQ ID NO: 1131), CAGguaaccg (SEQ ID NO: 1132), CAGguaacgu (SEQ ID NO: 1133), CAGguaacua (SEQ ID NO: 1134), CAGguaacuc (SEQ ID NO: 1135), CAGguaacug (SEQ ID NO: 1136), CAGguaacuu (SEQ ID NO: 1137), CAGguaagaa (SEQ ID NO: 1138), CAGguaagac (SEQ ID NO: 1139), CAGguaagag (SEQ ID NO: 1140), CAGguaagau (SEQ ID NO: 1141), CAGguaagcc (SEQ ID NO: 1142), CAGguaagga (SEQ ID NO: 1143), CAGguaaggc (SEQ ID NO: 1144), CAGguaaggg (SEQ ID NO: 1145), CAGguaaggu (SEQ ID NO: 1146), CAGguaagua (SEQ ID NO: 1147), CAGguaagug (SEQ ID NO: 1148), CAGguaaguu (SEQ ID NO: 1149), CAGguaauaa (SEQ ID NO: 1150), CAGguaauau (SEQ ID NO: 1151), CAGguaaucc (SEQ ID NO: 1152), CAGguaaugc (SEQ ID NO: 1153), CAGguaaugg (SEQ ID NO: 1154), CAGguaaugu (SEQ ID NO: 1155), CAGguaauua (SEQ ID NO: 1156), CAGguaauuc (SEQ ID NO: 1157), CAGguaauug (SEQ ID NO: 1158), CAGguaauuu (SEQ ID NO: 1159), CAGguacaaa (SEQ ID NO: 1160), CAGguacaag (SEQ ID NO: 1161), CAGguacaau (SEQ ID NO: 1162), CAGguacaca (SEQ ID NO: 1163), CAGguacacg (SEQ ID NO: 1164), CAGguacaga (SEQ ID NO: 1165), CAGguacagg (SEQ ID NO: 1166), CAGguacagu (SEQ ID NO: 1167), CAGguacaua (SEQ ID NO: 1168), CAGguacaug (SEQ ID NO: 1169), CAGguacauu (SEQ ID NO: 1170), CAGguaccac (SEQ ID NO: 1171), CAGguaccca (SEQ ID NO: 1172), CAGguacceg (SEQ ID NO: 1173), CAGguacccu (SEQ ID NO: 1174), CAGguacegc (SEQ ID NO: 1175), CAGguaccgg (SEQ ID NO: 1176), CAGguaccuc (SEQ ID NO: 1177), CAGguaccug (SEQ ID NO: 1178), CAGguaccuu (SEQ ID NO: 1179), CAGguacgag (SEQ ID NO: 1180), CAGguacgca (SEQ ID NO: 1181), CAGguacgcc (SEQ ID NO: 1182), CAGguacggu (SEQ ID NO: 1183), CAGguacgua (SEQ ID NO: 1184), CAGguacgug (SEQ ID NO: 1185), CAGguacuaa (SEQ ID NO: 1186), CAGguacuag (SEQ ID NO: 1187), CAGguacuau (SEQ ID NO: 1188), CAGguacucc (SEQ ID NO: 1189), CAGguacucu (SEQ ID NO: 1190), CAGguacuga (SEQ ID NO: 1191), CAGguacugc (SEQ ID NO: 1192), CAGguacugu (SEQ ID NO: 1193), CAGguacuua (SEQ ID NO: 1194), CAGguacuuu (SEQ ID NO: 1195), CAGguagaaa (SEQ ID NO: 1196), CAGguagaac (SEQ ID NO: 1197), CAGguagaag (SEQ ID NO: 1198), CAGguagaca (SEQ ID NO: 1199), CAGguagacc (SEQ ID NO: 1200), CAGguagaga (SEQ ID NO: 1201), CAGguagauu (SEQ ID NO: 1202), CAGguagcaa (SEQ ID NO: 1203), CAGguagcac (SEQ ID NO: 1204), CAGguagcag (SEQ ID NO: 1205), CAGguagcca (SEQ ID NO: 1206), CAGguagcgu (SEQ ID NO: 1207), CAGguagcua (SEQ ID NO: 1208), CAGguagcuc (SEQ ID NO: 1209), CAGguagcug (SEQ ID NO: 1210), CAGguagcuu (SEQ ID NO: 1211), CAGguaggaa (SEQ ID NO: 1212), CAGguaggac (SEQ ID NO: 1213), CAGguaggag (SEQ ID NO: 1214), CAGguaggca (SEQ ID NO: 1215), CAGguaggga (SEQ ID NO: 1216), CAGguagggc (SEQ ID NO: 1217), CAGguagggg (SEQ ID NO: 1218), CAGguagggu (SEQ ID NO: 1219), CAGguaggua (SEQ ID NO: 1220), CAGguagguc (SEQ ID NO: 1221), CAGguaggug (SEQ ID NO: 1222), CAGguagguu (SEQ ID NO: 1223), CAGguaguaa (SEQ ID NO: 1224), CAGguaguau (SEQ ID NO: 1225), CAGguaguca (SEQ ID NO: 1226), CAGguagucc (SEQ ID NO: 1227), CAGguaguga (SEQ ID NO: 1228), CAGguagugu (SEQ ID NO: 1229), CAGguaguuc (SEQ ID NO: 1230), CAGguaguug (SEQ ID NO: 1231), CAGguaguuu (SEQ ID NO: 1232), CAGguauaag (SEQ ID NO: 1233), CAGguauaca (SEQ ID NO: 1234), CAGguauaga (SEQ ID NO: 1235), CAGguauauc (SEQ ID NO: 1236), CAGguauaug (SEQ ID NO: 1237), CAGguauauu (SEQ ID NO: 1238), CAGguaucag (SEQ ID NO: 1239), CAGguaucau (SEQ ID NO: 1240), CAGguauccu (SEQ ID NO: 1241), CAGguaucga (SEQ ID NO: 1242), CAGguaucgc (SEQ ID NO: 1243), CAGguaucua (SEQ ID NO: 1244), CAGguaucug (SEQ ID NO: 1245), CAGguaucuu (SEQ ID NO: 1246), CAGguaugaa (SEQ ID NO: 1247), CAGguaugac (SEQ ID NO: 1248), CAGguaugag (SEQ ID NO: 1249), CAGguaugau (SEQ ID NO: 1250), CAGguaugca (SEQ ID NO: 1251), CAGguaugcc (SEQ ID NO: 1252), CAGguaugcg (SEQ ID NO: 1253), CAGguaugcu (SEQ ID NO: 1254), CAGguaugga (SEQ ID NO: 1255), CAGguauggg (SEQ ID NO: 1256), CAGguauggu (SEQ ID NO: 1257), CAGguaugua (SEQ ID NO: 1258), CAGguauguc (SEQ ID NO: 1259), CAGguaugug (SEQ ID NO: 1260), CAGguauguu (SEQ ID NO: 1261), CAGguauuau (SEQ ID NO: 1262), CAGguauuca (SEQ ID NO: 1263), CAGguauucu (SEQ ID NO: 1264), CAGguauuga (SEQ ID NO: 1265), CAGguauugg (SEQ ID NO: 1266), CAGguauugu (SEQ ID NO: 1267), CAGguauuua (SEQ ID NO: 1268), CAGguauuuc (SEQ ID NO: 1269), CAGguauuug (SEQ ID NO: 1270), CAGguauuuu (SEQ ID NO: 1271), CAGgucaaca (SEQ ID NO: 1272), CAGgucaaug (SEQ ID NO: 1273), CAGgucacgu (SEQ ID NO: 1274), CAGgucagaa (SEQ ID NO: 1275), CAGgucagac (SEQ ID NO: 1276), CAGgucagca (SEQ ID NO: 1277), CAGgucagcc (SEQ ID NO: 1278), CAGgucagcg (SEQ ID NO: 1279), CAGgucagga (SEQ ID NO: 1280), CAGgucagua (SEQ ID NO: 1281), CAGgucaguc (SEQ ID NO: 1282), CAGgucagug (SEQ ID NO: 1283), CAGgucaguu (SEQ ID NO: 1284), CAGgucaucc (SEQ ID NO: 1285), CAGgucaugc (SEQ ID NO: 1286), CAGgucauua (SEQ ID NO: 1287), CAGgucauuu (SEQ ID NO: 1288), CAGguccacc (SEQ ID NO: 1289), CAGguccacu (SEQ ID NO: 1290), CAGguccagu (SEQ ID NO: 1291), CAGguccauc (SEQ ID NO: 1292), CAGguccauu (SEQ ID NO: 1293), CAGgucccag (SEQ ID NO: 1294), CAGgucccug (SEQ ID NO: 1295), CAGguccuga (SEQ ID NO: 1296), CAGguccugc (SEQ ID NO: 1297), CAGguccugg (SEQ ID NO: 1298), CAGgucggcc (SEQ ID NO: 1299), CAGgucggug (SEQ ID NO: 1300), CAGgucguug (SEQ ID NO: 1301), CAGgucucuc (SEQ ID NO: 1302), CAGgucucuu (SEQ ID NO: 1303), CAGgucugag (SEQ ID NO: 1304), CAGgucugcc (SEQ ID NO: 1305), CAGgucugcg (SEQ ID NO: 1306), CAGgucugga (SEQ ID NO: 1307), CAGgucuggu (SEQ ID NO: 1308), CAGgucugua (SEQ ID NO: 1309), CAGgucuguc (SEQ ID NO: 1310), CAGgucugug (SEQ ID NO: 1311), CAGgucuguu (SEQ ID NO: 1312), CAGgucuucc (SEQ ID NO: 1313), CAGgucuuuc (SEQ ID NO: 1314), CAGgugaaag (SEQ ID NO: 1315), CAGgugaaau (SEQ ID NO: 1316), CAGgugaaca (SEQ ID NO: 1317), CAGgugaaga (SEQ ID NO: 1318), CAGgugaagg (SEQ ID NO: 1319), CAGgugaaua (SEQ ID NO: 1320), CAGgugaauc (SEQ ID NO: 1321), CAGgugaauu (SEQ ID NO: 1322), CAGgugacaa (SEQ ID NO: 1323), CAGgugacau (SEQ ID NO: 1324), CAGgugacca (SEQ ID NO: 1325), CAGgugaccc (SEQ ID NO: 1326), CAGgugaccg (SEQ ID NO: 1327), CAGgugaccu (SEQ ID NO: 1328), CAGgugacgg (SEQ ID NO: 1329), CAGgugacua (SEQ ID NO: 1330), CAGgugacuc (SEQ ID NO: 1331), CAGgugacug (SEQ ID NO: 1332), CAGgugagaa (SEQ ID NO: 1333), CAGgugagac (SEQ ID NO: 1334), CAGgugagag (SEQ ID NO: 1335), CAGgugagau (SEQ ID NO: 1336), CAGgugagca (SEQ ID NO: 1337), CAGgugagcc (SEQ ID NO: 1338), CAGgugageg (SEQ ID NO: 1339), CAGgugagcu (SEQ ID NO: 1340), CAGgugagga (SEQ ID NO: 1341), CAGgugaggc (SEQ ID NO: 1342), CAGgugaggg (SEQ ID NO: 1343), CAGgugaggu (SEQ ID NO: 1344), CAGgugagua (SEQ ID NO: 1345), CAGgugaguc (SEQ ID NO: 1346), CAGgugagug (SEQ ID NO: 1347), CAGgugaguu (SEQ ID NO: 1348), CAGgugauaa (SEQ ID NO: 1349), CAGgugaucc (SEQ ID NO: 1350), CAGgugaucu (SEQ ID NO: 1351), CAGgugaugc (SEQ ID NO: 1352), CAGgugaugg (SEQ ID NO: 1353), CAGgugaugu (SEQ ID NO: 1354), CAGgugauua (SEQ ID NO: 1355), CAGgugauuc (SEQ ID NO: 1356), CAGgugauug (SEQ ID NO: 1357), CAGgugauuu (SEQ ID NO: 1358), CAGgugcaaa (SEQ ID NO: 1359), CAGgugcaag (SEQ ID NO: 1360), CAGgugcaca (SEQ ID NO: 1361), CAGgugcacg (SEQ ID NO: 1362), CAGgugcaga (SEQ ID NO: 1363), CAGgugcagg (SEQ ID NO: 1364), CAGgugcaua (SEQ ID NO: 1365), CAGgugcauc (SEQ ID NO: 1366), CAGgugcaug (SEQ ID NO: 1367), CAGgugccaa (SEQ ID NO: 1368), CAGgugccca (SEQ ID NO: 1369), CAGgugcccc (SEQ ID NO: 1370), CAGgugcccg (SEQ ID NO: 1371), CAGgugccua (SEQ ID NO: 1372), CAGgugccug (SEQ ID NO: 1373), CAGgugcgaa (SEQ ID NO: 1374), CAGgugcgca (SEQ ID NO: 1375), CAGgugegcc (SEQ ID NO: 1376), CAGgugcgcg (SEQ ID NO: 1377), CAGgugegga (SEQ ID NO: 1378), CAGgugcggu (SEQ ID NO: 1379), CAGgugegua (SEQ ID NO: 1380), CAGgugeguc (SEQ ID NO: 1381), CAGgugcgug (SEQ ID NO: 1382), CAGgugcuag (SEQ ID NO: 1383), CAGgugcuau (SEQ ID NO: 1384), CAGgugcuca (SEQ ID NO: 1385), CAGgugcucc (SEQ ID NO: 1386), CAGgugcucg (SEQ ID NO: 1387), CAGgugcugc (SEQ ID NO: 1388), CAGgugcugg (SEQ ID NO: 1389), CAGgugcuua (SEQ ID NO: 1390), CAGgugcuuc (SEQ ID NO: 1391), CAGgugcuug (SEQ ID NO: 1392), CAGguggaac (SEQ ID NO: 1393), CAGguggaag (SEQ ID NO: 1394), CAGguggaau (SEQ ID NO: 1395), CAGguggaga (SEQ ID NO: 1396), CAGguggagu (SEQ ID NO: 1397), CAGguggauu (SEQ ID NO: 1398), CAGguggcca (SEQ ID NO: 1399), CAGguggcuc (SEQ ID NO: 1400), CAGguggcug (SEQ ID NO: 1401), CAGgugggaa (SEQ ID NO: 1402), CAGgugggac (SEQ ID NO: 1403), CAGgugggag (SEQ ID NO: 1404), CAGgugggau (SEQ ID NO: 1405), CAGgugggca (SEQ ID NO: 1406), CAGgugggcc (SEQ ID NO: 1407), CAGgugggcu (SEQ ID NO: 1408), CAGgugggga (SEQ ID NO: 1409), CAGguggggc (SEQ ID NO: 1410), CAGguggggg (SEQ ID NO: 1411), CAGguggggu (SEQ ID NO: 1412), CAGgugggua (SEQ ID NO: 1413), CAGguggguc (SEQ ID NO: 1414), CAGgugggug (SEQ ID NO: 1415), CAGguggguu (SEQ ID NO: 1416), CAGguggucu (SEQ ID NO: 1417), CAGguggugg (SEQ ID NO: 1418), CAGgugguug (SEQ ID NO: 1419), CAGguguaca (SEQ ID NO: 1420), CAGguguagg (SEQ ID NO: 1421), CAGguguauc (SEQ ID NO: 1422), CAGgugucac (SEQ ID NO: 1423), CAGgugucag (SEQ ID NO: 1424), CAGgugucca (SEQ ID NO: 1425), CAGguguccu (SEQ ID NO: 1426), CAGgugucua (SEQ ID NO: 1427), CAGgugucuc (SEQ ID NO: 1428), CAGgugucug (SEQ ID NO: 1429), CAGgugugaa (SEQ ID NO: 1430), CAGgugugac (SEQ ID NO: 1431), CAGgugugag (SEQ ID NO: 1432), CAGgugugau (SEQ ID NO: 1433), CAGgugugca (SEQ ID NO: 1434), CAGgugugcc (SEQ ID NO: 1435), CAGgugugcg (SEQ ID NO: 1436), CAGgugugcu (SEQ ID NO: 1437), CAGgugugga (SEQ ID NO: 1438), CAGguguggc (SEQ ID NO: 1439), CAGgugugua (SEQ ID NO: 1440), CAGguguguc (SEQ ID NO: 1441), CAGgugugug (SEQ ID NO: 1442), CAGguguguu (SEQ ID NO: 1443), CAGguguuua (SEQ ID NO: 1444), CAGguuaaaa (SEQ ID NO: 1445), CAGguuaaua (SEQ ID NO: 1446), CAGguuaauc (SEQ ID NO: 1447), CAGguuaccu (SEQ ID NO: 1448), CAGguuagaa (SEQ ID NO: 1449), CAGguuagag (SEQ ID NO: 1450), CAGguuagau (SEQ ID NO: 1451), CAGguuagcc (SEQ ID NO: 1452), CAGguuaggg (SEQ ID NO: 1453), CAGguuaggu (SEQ ID NO: 1454), CAGguuagua (SEQ ID NO: 1455), CAGguuaguc (SEQ ID NO: 1456), CAGguuagug (SEQ ID NO: 1457), CAGguuaguu (SEQ ID NO: 1458), CAGguuauca (SEQ ID NO: 1459), CAGguuaugu (SEQ ID NO: 1460), CAGguuauua (SEQ ID NO: 1461), CAGguuauug (SEQ ID NO: 1462), CAGguucaaa (SEQ ID NO: 1463), CAGguucaac (SEQ ID NO: 1464), CAGguucaag (SEQ ID NO: 1465), CAGguucaca (SEQ ID NO: 1466), CAGguucacg (SEQ ID NO: 1467), CAGguucagg (SEQ ID NO: 1468), CAGguucaug (SEQ ID NO: 1469), CAGguuccag (SEQ ID NO: 1470), CAGguuccca (SEQ ID NO: 1471), CAGguucceg (SEQ ID NO: 1472), CAGguucgaa (SEQ ID NO: 1473), CAGguucgag (SEQ ID NO: 1474), CAGguucuau (SEQ ID NO: 1475), CAGguucugc (SEQ ID NO: 1476), CAGguucuua (SEQ ID NO: 1477), CAGguucuuc (SEQ ID NO: 1478), CAGguucuuu (SEQ ID NO: 1479), CAGguugaac (SEQ ID NO: 1480), CAGguugaag (SEQ ID NO: 1481), CAGguugagu (SEQ ID NO: 1482), CAGguugaua (SEQ ID NO: 1483), CAGguuggag (SEQ ID NO: 1484), CAGguuggca (SEQ ID NO: 1485), CAGguuggcc (SEQ ID NO: 1486), CAGguugguc (SEQ ID NO: 1487), CAGguuggug (SEQ ID NO: 1488), CAGguugguu (SEQ ID NO: 1489), CAGguuguaa (SEQ ID NO: 1490), CAGguuguac (SEQ ID NO: 1491), CAGguuguau (SEQ ID NO: 1492), CAGguuguca (SEQ ID NO: 1493), CAGguuguga (SEQ ID NO: 1494), CAGguuguug (SEQ ID NO: 1495), CAGguuuaag (SEQ ID NO: 1496), CAGguuuacc (SEQ ID NO: 1497), CAGguuuagc (SEQ ID NO: 1498), CAGguuuagu (SEQ ID NO: 1499), CAGguuucuu (SEQ ID NO: 1500), CAGguuugaa (SEQ ID NO: 1501), CAGguuugag (SEQ ID NO: 1502), CAGguuugau (SEQ ID NO: 1503), CAGguuugcc (SEQ ID NO: 1504), CAGguuugcu (SEQ ID NO: 1505), CAGguuuggg (SEQ ID NO: 1506), CAGguuuggu (SEQ ID NO: 1507), CAGguuugua (SEQ ID NO: 1508), CAGguuugug (SEQ ID NO: 1509), CAGguuuguu (SEQ ID NO: 1510), CAGguuuucu (SEQ ID NO: 1511), CAGguuuugg (SEQ ID NO: 1512), CAGguuuuuc (SEQ ID NO: 1513), CAGguuuuuu (SEQ ID NO: 1514), CAUgcagguu (SEQ ID NO: 1515), CAUguaaaac (SEQ ID NO: 1516), CAUguaacua (SEQ ID NO: 1517), CAUguaagaa (SEQ ID NO: 1518), CAUguaagag (SEQ ID NO: 1519), CAUguaagau (SEQ ID NO: 1520), CAUguaagcc (SEQ ID NO: 1521), CAUguaagua (SEQ ID NO: 1522), CAUguaagug (SEQ ID NO: 1523), CAUguaaguu (SEQ ID NO: 1524), CAUguaauua (SEQ ID NO: 1525), CAUguacaua (SEQ ID NO: 1526), CAUguaccac (SEQ ID NO: 1527), CAUguacguu (SEQ ID NO: 1528), CAUguaggua (SEQ ID NO: 1529), CAUguaggug (SEQ ID NO: 1530), CAUguagguu (SEQ ID NO: 1531), CAUguaugaa (SEQ ID NO: 1532), CAUguaugua (SEQ ID NO: 1533), CAUguaugug (SEQ ID NO: 1534), CAUguauguu (SEQ ID NO: 1535), CAUgugagaa (SEQ ID NO: 1536), CAUgugagca (SEQ ID NO: 1537), CAUgugagcu (SEQ ID NO: 1538), CAUgugagua (SEQ ID NO: 1539), CAUgugaguc (SEQ ID NO: 1540), CAUgugagug (SEQ ID NO: 1541), CAUgugaguu (SEQ ID NO: 1542), CAUgugegua (SEQ ID NO: 1543), CAUgugggaa (SEQ ID NO: 1544), CAUguggguu (SEQ ID NO: 1545), CAUgugugug (SEQ ID NO: 1546), CAUguguguu (SEQ ID NO: 1547), CAUguuaaua (SEQ ID NO: 1548), CAUguuagcc (SEQ ID NO: 1549), CCAguaagau (SEQ ID NO: 1550), CCAguaagca (SEQ ID NO: 1551), CCAguaagcc (SEQ ID NO: 1552), CCAguaagcu (SEQ ID NO: 1553), CCAguaagga (SEQ ID NO: 1554), CCAguaagua (SEQ ID NO: 1555), CCAguaaguc (SEQ ID NO: 1556), CCAguaagug (SEQ ID NO: 1557), CCAguaaguu (SEQ ID NO: 1558), CCAguaauug (SEQ ID NO: 1559), CCAguacggg (SEQ ID NO: 1560), CCAguagguc (SEQ ID NO: 1561), CCAguauugu (SEQ ID NO: 1562), CCAgugaggc (SEQ ID NO: 1563), CCAgugagua (SEQ ID NO: 1564), CCAgugagug (SEQ ID NO: 1565), CCAguggguc (SEQ ID NO: 1566), CCAguuaguu (SEQ ID NO: 1567), CCAguugagu (SEQ ID NO: 1568), CCCguaagau (SEQ ID NO: 1569), CCCguauguc (SEQ ID NO: 1570), CCCguauguu (SEQ ID NO: 1571), CCCguccugc (SEQ ID NO: 1572), CCCgugagug (SEQ ID NO: 1573), CCGguaaaga (SEQ ID NO: 1574), CCGguaagau (SEQ ID NO: 1575), CCGguaagcc (SEQ ID NO: 1576), CCGguaagga (SEQ ID NO: 1577), CCGguaaggc (SEQ ID NO: 1578), CCGguaaugg (SEQ ID NO: 1579), CCGguacagu (SEQ ID NO: 1580), CCGguacuga (SEQ ID NO: 1581), CCGguauucc (SEQ ID NO: 1582), CCGgucagug (SEQ ID NO: 1583), CCGgugaaaa (SEQ ID NO: 1584), CCGgugagaa (SEQ ID NO: 1585), CCGgugaggg (SEQ ID NO: 1586), CCGgugagug (SEQ ID NO: 1587), CCGgugaguu (SEQ ID NO: 1588), CCGgugegeg (SEQ ID NO: 1589), CCGgugggeg (SEQ ID NO: 1590), CCGguugguc (SEQ ID NO: 1591), CCUguaaaug (SEQ ID NO: 1592), CCUguaaauu (SEQ ID NO: 1593), CCUguagaa (SEQ ID NO: 1594), CCUguagac (SEQ ID NO: 1595), CCUguagag (SEQ ID NO: 1596), CCUguagca (SEQ ID NO: 1597), CCUguageg (SEQ ID NO: 1598), CCUguaagga (SEQ ID NO: 1599), CCUguaguu (SEQ ID NO: 1600), CCUguaggua (SEQ ID NO: 1601), CCUguaggug (SEQ ID NO: 1602), CCUguaucuu (SEQ ID NO: 1603), CCUguauggu (SEQ ID NO: 1604), CCUguaugug (SEQ ID NO: 1605), CCUgugaga (SEQ ID NO: 1606), CCUgugagca (SEQ ID NO: 1607), CCUgugaggg (SEQ ID NO: 1608), CCUgugaguc (SEQ ID NO: 1609), CCUgugagug (SEQ ID NO: 1610), CCUgugaguu (SEQ ID NO: 1611), CCUguggcuc (SEQ ID NO: 1612), CCUgugggua (SEQ ID NO: 1613), CCUgugugua (SEQ ID NO: 1614), CCUguuagaa (SEQ ID NO: 1615), CGAguaaggg (SEQ ID NO: 1616), CGAguaaggu (SEQ ID NO: 1617), CGAguagcug (SEQ ID NO: 1618), CGAguaggug (SEQ ID NO: 1619), CGAguagguu (SEQ ID NO: 1620), CGAgugagca (SEQ ID NO: 1621), CGCguagag (SEQ ID NO: 1622), CGGgcaggca (SEQ ID NO: 1623), CGGguagce (SEQ ID NO: 1624), CGGguagcu (SEQ ID NO: 1625), CGGguaguu (SEQ ID NO: 1626), CGGguaauc (SEQ ID NO: 1627), CGGguaauuu (SEQ ID NO: 1628), CGGguacagu (SEQ ID NO: 1629), CGGguacggg (SEQ ID NO: 1630), CGGguaggag (SEQ ID NO: 1631), CGGguaggee (SEQ ID NO: 1632), CGGguaggug (SEQ ID NO: 1633), CGGguauuua (SEQ ID NO: 1634), CGGgucugag (SEQ ID NO: 1635), CGGgugaccg (SEQ ID NO: 1636), CGGgugacuc (SEQ ID NO: 1637), CGGgugagaa (SEQ ID NO: 1638), CGGgugaggg (SEQ ID NO: 1639), CGGgugaggu (SEQ ID NO: 1640), CGGgugagua (SEQ ID NO: 1641), CGGgugagug (SEQ ID NO: 1642), CGGgugaguu (SEQ ID NO: 1643), CGGgugauuu (SEQ ID NO: 1644), CGGgugecuu (SEQ ID NO: 1645), CGGgugggag (SEQ ID NO: 1646), CGGgugggug (SEQ ID NO: 1647), CGGguggguu (SEQ ID NO: 1648), CGGguguguc (SEQ ID NO: 1649), CGGgugugug (SEQ ID NO: 1650), CGGguguguu (SEQ ID NO: 1651), CGGguucaag (SEQ ID NO: 1652), CGGguucaug (SEQ ID NO: 1653), CGGguuugcu (SEQ ID NO: 1654), CGUguagggu (SEQ ID NO: 1655), CGUguaugca (SEQ ID NO: 1656), CGUguaugua (SEQ ID NO: 1657), CGUgucugua (SEQ ID NO: 1658), CGUgugagug (SEQ ID NO: 1659), CGUguuuucu (SEQ ID NO: 1660), CUAguaaug (SEQ ID NO: 1661), CUAguageg (SEQ ID NO: 1662), CUAguagcu (SEQ ID NO: 1663), CUAguaagua (SEQ ID NO: 1664), CUAguaguc (SEQ ID NO: 1665), CUAguagug (SEQ ID NO: 1666), CUAguaaguu (SEQ ID NO: 1667), CUAguaauuu (SEQ ID NO: 1668), CUAguaggua (SEQ ID NO: 1669), CUAguagguu (SEQ ID NO: 1670), CUAguaugua (SEQ ID NO: 1671), CUAguauguu (SEQ ID NO: 1672), CUAgugagua (SEQ ID NO: 1673), CUCguaagca (SEQ ID NO: 1674), CUCguaagug (SEQ ID NO: 1675), CUCguaaguu (SEQ ID NO: 1676), CUCguaucug (SEQ ID NO: 1677), CUCgucugug (SEQ ID NO: 1678), CUCgugaaua (SEQ ID NO: 1679), CUCgugagua (SEQ ID NO: 1680), CUCgugauua (SEQ ID NO: 1681), CUGguaaaaa (SEQ ID NO: 1682), CUGguaaaau (SEQ ID NO: 1683), CUGguaaacc (SEQ ID NO: 1684), CUGguaaacg (SEQ ID NO: 1685), CUGguaaagc (SEQ ID NO: 1686), CUGguaaaua (SEQ ID NO: 1687), CUGguaaauc (SEQ ID NO: 1688), CUGguaaaug (SEQ ID NO: 1689), CUGguaaauu (SEQ ID NO: 1690), CUGguaacac (SEQ ID NO: 1691), CUGguaacag (SEQ ID NO: 1692), CUGguaaccc (SEQ ID NO: 1693), CUGguaaccg (SEQ ID NO: 1694), CUGguaacug (SEQ ID NO: 1695), CUGguaacuu (SEQ ID NO: 1696), CUGguaagaa (SEQ ID NO: 1697), CUGguaagag (SEQ ID NO: 1698), CUGguaagau (SEQ ID NO: 1699), CUGguaagca (SEQ ID NO: 1700), CUGguaagcc (SEQ ID NO: 1701), CUGguaagcu (SEQ ID NO: 1702), CUGguaagga (SEQ ID NO: 1703), CUGguaaggc (SEQ ID NO: 1704), CUGguaaggg (SEQ ID NO: 1705), CUGguaaggu (SEQ ID NO: 1706), CUGguaagua (SEQ ID NO: 1707), CUGguaagug (SEQ ID NO: 1708), CUGguaaguu (SEQ ID NO: 1709), CUGguaauga (SEQ ID NO: 1710), CUGguaaugc (SEQ ID NO: 1711), CUGguaauuc (SEQ ID NO: 1712), CUGguaauuu (SEQ ID NO: 1713), CUGguacaac (SEQ ID NO: 1714), CUGguacaau (SEQ ID NO: 1715), CUGguacaga (SEQ ID NO: 1716), CUGguacaua (SEQ ID NO: 1717), CUGguacauu (SEQ ID NO: 1718), CUGguaccau (SEQ ID NO: 1719), CUGguacguu (SEQ ID NO: 1720), CUGguacuaa (SEQ ID NO: 1721), CUGguacuug (SEQ ID NO: 1722), CUGguacuuu (SEQ ID NO: 1723), CUGguagaga (SEQ ID NO: 1724), CUGguagaua (SEQ ID NO: 1725), CUGguagcgu (SEQ ID NO: 1726), CUGguaggau (SEQ ID NO: 1727), CUGguaggca (SEQ ID NO: 1728), CUGguaggua (SEQ ID NO: 1729), CUGguagguc (SEQ ID NO: 1730), CUGguaggug (SEQ ID NO: 1731), CUGguaucaa (SEQ ID NO: 1732), CUGguaugau (SEQ ID NO: 1733), CUGguauggc (SEQ ID NO: 1734), CUGguauggu (SEQ ID NO: 1735), CUGguaugua (SEQ ID NO: 1736), CUGguaugug (SEQ ID NO: 1737), CUGguauguu (SEQ ID NO: 1738), CUGguauuga (SEQ ID NO: 1739), CUGguauuuc (SEQ ID NO: 1740), CUGguauuuu (SEQ ID NO: 1741), CUGgucaaca (SEQ ID NO: 1742), CUGgucagag (SEQ ID NO: 1743), CUGguccegc (SEQ ID NO: 1744), CUGgucggua (SEQ ID NO: 1745), CUGgucuggg (SEQ ID NO: 1746), CUGgugaagu (SEQ ID NO: 1747), CUGgugaaua (SEQ ID NO: 1748), CUGgugaauu (SEQ ID NO: 1749), CUGgugacua (SEQ ID NO: 1750), CUGgugagaa (SEQ ID NO: 1751), CUGgugagac (SEQ ID NO: 1752), CUGgugagca (SEQ ID NO: 1753), CUGgugagcu (SEQ ID NO: 1754), CUGgugagga (SEQ ID NO: 1755), CUGgugaggc (SEQ ID NO: 1756), CUGgugaggg (SEQ ID NO: 1757), CUGgugaggu (SEQ ID NO: 1758), CUGgugagua (SEQ ID NO: 1759), CUGgugaguc (SEQ ID NO: 1760), CUGgugagug (SEQ ID NO: 1761), CUGgugaguu (SEQ ID NO: 1762), CUGgugauua (SEQ ID NO: 1763), CUGgugauuu (SEQ ID NO: 1764), CUGgugcaga (SEQ ID NO: 1765), CUGgugcgcu (SEQ ID NO: 1766), CUGgugcgug (SEQ ID NO: 1767), CUGgugcuga (SEQ ID NO: 1768), CUGgugggag (SEQ ID NO: 1769), CUGgugggga (SEQ ID NO: 1770), CUGgugggua (SEQ ID NO: 1771), CUGguggguc (SEQ ID NO: 1772), CUGgugggug (SEQ ID NO: 1773), CUGguggguu (SEQ ID NO: 1774), CUGgugugaa (SEQ ID NO: 1775), CUGgugugca (SEQ ID NO: 1776), CUGgugugcu (SEQ ID NO: 1777), CUGguguggu (SEQ ID NO: 1778), CUGgugugug (SEQ ID NO: 1779), CUGguguguu (SEQ ID NO: 1780), CUGguuagcu (SEQ ID NO: 1781), CUGguuagug (SEQ ID NO: 1782), CUGguucgug (SEQ ID NO: 1783), CUGguuggcu (SEQ ID NO: 1784), CUGguuguuu (SEQ ID NO: 1785), CUGguuugua (SEQ ID NO: 1786), CUGguuuguc (SEQ ID NO: 1787), CUGguuugug (SEQ ID NO: 1788), CUUguaaaug (SEQ ID NO: 1789), CUUguaagcu (SEQ ID NO: 1790), CUUguaagga (SEQ ID NO: 1791), CUUguaaggc (SEQ ID NO: 1792), CUUguaagua (SEQ ID NO: 1793), CUUguaagug (SEQ ID NO: 1794), CUUguaaguu (SEQ ID NO: 1795), CUUguacguc (SEQ ID NO: 1796), CUUguacgug (SEQ ID NO: 1797), CUUguaggua (SEQ ID NO: 1798), CUUguagugc (SEQ ID NO: 1799), CUUguauagg (SEQ ID NO: 1800), CUUgucagua (SEQ ID NO: 1801), CUUgugagua (SEQ ID NO: 1802), CUUgugaguc (SEQ ID NO: 1803), CUUgugaguu (SEQ ID NO: 1804), CUUguggguu (SEQ ID NO: 1805), CUUgugugua (SEQ ID NO: 1806), CUUguuagug (SEQ ID NO: 1807), CUUguuugag (SEQ ID NO: 1808), GAAguaaaac (SEQ ID NO: 1809), GAAguaaagc (SEQ ID NO: 1810), GAAguaaagu (SEQ ID NO: 1811), GAAguaaaua (SEQ ID NO: 1812), GAAguaaauu (SEQ ID NO: 1813), GAAguaagaa (SEQ ID NO: 1814), GAAguaagcc (SEQ ID NO: 1815), GAAguaagcu (SEQ ID NO: 1816), GAAguaagga (SEQ ID NO: 1817), GAAguaagua (SEQ ID NO: 1818), GAAguaagug (SEQ ID NO: 1819), GAAguaaguu (SEQ ID NO: 1820), GAAguaauau (SEQ ID NO: 1821), GAAguaaugc (SEQ ID NO: 1822), GAAguaauua (SEQ ID NO: 1823), GAAguaauuu (SEQ ID NO: 1824), GAAguaccau (SEQ ID NO: 1825), GAAguacgua (SEQ ID NO: 1826), GAAguacguc (SEQ ID NO: 1827), GAAguaggca (SEQ ID NO: 1828), GAAguagguc (SEQ ID NO: 1829), GAAguauaaa (SEQ ID NO: 1830), GAAguaugcu (SEQ ID NO: 1831), GAAguaugug (SEQ ID NO: 1832), GAAguauguu (SEQ ID NO: 1833), GAAguauuaa (SEQ ID NO: 1834), GAAgucagug (SEQ ID NO: 1835), GAAgugagag (SEQ ID NO: 1836), GAAgugagcg (SEQ ID NO: 1837), GAAgugaggu (SEQ ID NO: 1838), GAAgugaguc (SEQ ID NO: 1839), GAAgugagug (SEQ ID NO: 1840), GAAgugaguu (SEQ ID NO: 1841), GAAgugauaa (SEQ ID NO: 1842), GAAgugauuc (SEQ ID NO: 1843), GAAgugcgug (SEQ ID NO: 1844), GAAguguggg (SEQ ID NO: 1845), GAAguguguc (SEQ ID NO: 1846), GAAguuggug (SEQ ID NO: 1847), GACguaaagu (SEQ ID NO: 1848), GACguaagcu (SEQ ID NO: 1849), GACguaagua (SEQ ID NO: 1850), GACguaaugg (SEQ ID NO: 1851), GACguaugcc (SEQ ID NO: 1852), GACguauguu (SEQ ID NO: 1853), GACgugagcc (SEQ ID NO: 1854), GACgugagug (SEQ ID NO: 1855), GAGgcaaaug (SEQ ID NO: 1856), GAGgcaagag (SEQ ID NO: 1857), GAGgcaagua (SEQ ID NO: 1858), GAGgcaagug (SEQ ID NO: 1859), GAGgcaaguu (SEQ ID NO: 1860), GAGgcacgag (SEQ ID NO: 1861), GAGgcaggga (SEQ ID NO: 1862), GAGgcaugug (SEQ ID NO: 1863), GAGgcgaagg (SEQ ID NO: 1864), GAGguaaaaa (SEQ ID NO: 1865), GAGguaaaac (SEQ ID NO: 1866), GAGguaaaag (SEQ ID NO: 1867), GAGguaaaau (SEQ ID NO: 1868), GAGguaaacc (SEQ ID NO: 1869), GAGguaaaga (SEQ ID NO: 1870), GAGguaaagc (SEQ ID NO: 1871), GAGguaaagu (SEQ ID NO: 1872), GAGguaaaua (SEQ ID NO: 1873), GAGguaaauc (SEQ ID NO: 1874), GAGguaaaug (SEQ ID NO: 1875), GAGguaaauu (SEQ ID NO: 1876), GAGguaacaa (SEQ ID NO: 1877), GAGguaacag (SEQ ID NO: 1878), GAGguaacca (SEQ ID NO: 1879), GAGguaaccu (SEQ ID NO: 1880), GAGguaacuu (SEQ ID NO: 1881), GAGguaagaa (SEQ ID NO: 1882), GAGguaagag (SEQ ID NO: 1883), GAGguaagau (SEQ ID NO: 1884), GAGguaagca (SEQ ID NO: 1885), GAGguaagcc (SEQ ID NO: 1886), GAGguaagcg (SEQ ID NO: 1887), GAGguaagcu (SEQ ID NO: 1888), GAGguaagga (SEQ ID NO: 1889), GAGguaaggc (SEQ ID NO: 1890), GAGguaaggg (SEQ ID NO: 1891), GAGguaaggu (SEQ ID NO: 1892), GAGguaagua (SEQ ID NO: 1893), GAGguaaguc (SEQ ID NO: 1894), GAGguaauaa (SEQ ID NO: 1895), GAGguaauac (SEQ ID NO: 1896), GAGguaauau (SEQ ID NO: 1897), GAGguaauca (SEQ ID NO: 1898), GAGguaaucu (SEQ ID NO: 1899), GAGguaaugg (SEQ ID NO: 1900), GAGguaaugu (SEQ ID NO: 1901), GAGguaauug (SEQ ID NO: 1902), GAGguaauuu (SEQ ID NO: 1903), GAGguacaaa (SEQ ID NO: 1904), GAGguacaac (SEQ ID NO: 1905), GAGguacaga (SEQ ID NO: 1906), GAGguacagc (SEQ ID NO: 1907), GAGguacagu (SEQ ID NO: 1908), GAGguacaua (SEQ ID NO: 1909), GAGguacauu (SEQ ID NO: 1910), GAGguaccag (SEQ ID NO: 1911), GAGguaccga (SEQ ID NO: 1912), GAGguaccug (SEQ ID NO: 1913), GAGguaccuu (SEQ ID NO: 1914), GAGguacuag (SEQ ID NO: 1915), GAGguacuau (SEQ ID NO: 1916), GAGguacucc (SEQ ID NO: 1917), GAGguacugc (SEQ ID NO: 1918), GAGguacugg (SEQ ID NO: 1919), GAGguacugu (SEQ ID NO: 1920), GAGguacuug (SEQ ID NO: 1921), GAGguacuuu (SEQ ID NO: 1922), GAGguagaag (SEQ ID NO: 1923), GAGguagaga (SEQ ID NO: 1924), GAGguagagg (SEQ ID NO: 1925), GAGguagagu (SEQ ID NO: 1926), GAGguagauc (SEQ ID NO: 1927), GAGguagcua (SEQ ID NO: 1928), GAGguagcug (SEQ ID NO: 1929), GAGguaggaa (SEQ ID NO: 1930), GAGguaggag (SEQ ID NO: 1931), GAGguaggca (SEQ ID NO: 1932), GAGguaggcu (SEQ ID NO: 1933), GAGguaggga (SEQ ID NO: 1934), GAGguagggc (SEQ ID NO: 1935), GAGguagggg (SEQ ID NO: 1936), GAGguaggua (SEQ ID NO: 1937), GAGguaggug (SEQ ID NO: 1938), GAGguagguu (SEQ ID NO: 1939), GAGguaguaa (SEQ ID NO: 1940), GAGguaguag (SEQ ID NO: 1941), GAGguaguau (SEQ ID NO: 1942), GAGguagucu (SEQ ID NO: 1943), GAGguagugc (SEQ ID NO: 1944), GAGguagugg (SEQ ID NO: 1945), GAGguaguua (SEQ ID NO: 1946), GAGguaguug (SEQ ID NO: 1947), GAGguauaag (SEQ ID NO: 1948), GAGguauacu (SEQ ID NO: 1949), GAGguauagc (SEQ ID NO: 1950), GAGguauaug (SEQ ID NO: 1951), GAGguauauu (SEQ ID NO: 1952), GAGguaucau (SEQ ID NO: 1953), GAGguaucug (SEQ ID NO: 1954), GAGguaucuu (SEQ ID NO: 1955), GAGguaugaa (SEQ ID NO: 1956), GAGguaugac (SEQ ID NO: 1957), GAGguaugag (SEQ ID NO: 1958), GAGguaugcc (SEQ ID NO: 1959), GAGguaugcg (SEQ ID NO: 1960), GAGguaugcu (SEQ ID NO: 1961), GAGguaugga (SEQ ID NO: 1962), GAGguauggg (SEQ ID NO: 1963), GAGguauggu (SEQ ID NO: 1964), GAGguaugua (SEQ ID NO: 1965), GAGguauguc (SEQ ID NO: 1966), GAGguaugug (SEQ ID NO: 1967), GAGguauguu (SEQ ID NO: 1968), GAGguauucc (SEQ ID NO: 1969), GAGguauuga (SEQ ID NO: 1970), GAGguauugu (SEQ ID NO: 1971), GAGguauuua (SEQ ID NO: 1972), GAGguauuuc (SEQ ID NO: 1973), GAGguauuug (SEQ ID NO: 1974), GAGguauuuu (SEQ ID NO: 1975), GAGgucaaca (SEQ ID NO: 1976), GAGgucaagg (SEQ ID NO: 1977), GAGgucaaug (SEQ ID NO: 1978), GAGgucacug (SEQ ID NO: 1979), GAGgucagaa (SEQ ID NO: 1980), GAGgucagag (SEQ ID NO: 1981), GAGgucagcu (SEQ ID NO: 1982), GAGgucagga (SEQ ID NO: 1983), GAGgucaggc (SEQ ID NO: 1984), GAGgucaggg (SEQ ID NO: 1985), GAGgucaggu (SEQ ID NO: 1986), GAGgucagua (SEQ ID NO: 1987), GAGgucauau (SEQ ID NO: 1988), GAGgucaugu (SEQ ID NO: 1989), GAGgucauuu (SEQ ID NO: 1990), GAGguccaua (SEQ ID NO: 1991), GAGguccauc (SEQ ID NO: 1992), GAGguccggg (SEQ ID NO: 1993), GAGguccggu (SEQ ID NO: 1994), GAGguccuug (SEQ ID NO: 1995), GAGgucgggg (SEQ ID NO: 1996), GAGgucucgu (SEQ ID NO: 1997), GAGgucugag (SEQ ID NO: 1998), GAGgucuggu (SEQ ID NO: 1999), GAGgucuguc (SEQ ID NO: 2000), GAGgucuguu (SEQ ID NO: 2001), GAGgucuuuu (SEQ ID NO: 2002), GAGgugaaaa (SEQ ID NO: 2003), GAGgugaaau (SEQ ID NO: 2004), GAGgugaaca (SEQ ID NO: 2005), GAGgugaagg (SEQ ID NO: 2006), GAGgugaaua (SEQ ID NO: 2007), GAGgugaauu (SEQ ID NO: 2008), GAGgugacau (SEQ ID NO: 2009), GAGgugacca (SEQ ID NO: 2010), GAGgugaccu (SEQ ID NO: 2011), GAGgugacua (SEQ ID NO: 2012), GAGgugacuu (SEQ ID NO: 2013), GAGgugagaa (SEQ ID NO: 2014), GAGgugagac (SEQ ID NO: 2015), GAGgugagag (SEQ ID NO: 2016), GAGgugagau (SEQ ID NO: 2017), GAGgugagca (SEQ ID NO: 2018), GAGgugagcc (SEQ ID NO: 2019), GAGgugageg (SEQ ID NO: 2020), GAGgugagcu (SEQ ID NO: 2021), GAGgugagga (SEQ ID NO: 2022), GAGgugaggc (SEQ ID NO: 2023), GAGgugaggg (SEQ ID NO: 2024), GAGgugagua (SEQ ID NO: 2025), GAGgugagug (SEQ ID NO: 2026), GAGgugaguu (SEQ ID NO: 2027), GAGgugauau (SEQ ID NO: 2028), GAGgugaucc (SEQ ID NO: 2029), GAGgugaucu (SEQ ID NO: 2030), GAGgugauga (SEQ ID NO: 2031), GAGgugaugg (SEQ ID NO: 2032), GAGgugaugu (SEQ ID NO: 2033), GAGgugauuc (SEQ ID NO: 2034), GAGgugcaca (SEQ ID NO: 2035), GAGgugcaga (SEQ ID NO: 2036), GAGgugcagc (SEQ ID NO: 2037), GAGgugcagg (SEQ ID NO: 2038), GAGgugccag (SEQ ID NO: 2039), GAGgugccca (SEQ ID NO: 2040), GAGgugccuu (SEQ ID NO: 2041), GAGgugggg (SEQ ID NO: 2042), GAGgugcgug (SEQ ID NO: 2043), GAGgugcucc (SEQ ID NO: 2044), GAGgugcugg (SEQ ID NO: 2045), GAGgugcuua (SEQ ID NO: 2046), GAGgugcuug (SEQ ID NO: 2047), GAGguggaaa (SEQ ID NO: 2048), GAGguggaau (SEQ ID NO: 2049), GAGguggacc (SEQ ID NO: 2050), GAGguggacg (SEQ ID NO: 2051), GAGguggagg (SEQ ID NO: 2052), GAGguggcug (SEQ ID NO: 2053), GAGgugggaa (SEQ ID NO: 2054), GAGgugggag (SEQ ID NO: 2055), GAGgugggau (SEQ ID NO: 2056), GAGgugggca (SEQ ID NO: 2057), GAGgugggcg (SEQ ID NO: 2058), GAGgugggcu (SEQ ID NO: 2059), GAGgugggga (SEQ ID NO: 2060), GAGguggggc (SEQ ID NO: 2061), GAGguggggg (SEQ ID NO: 2062), GAGgugggua (SEQ ID NO: 2063), GAGguggguc (SEQ ID NO: 2064), GAGgugggug (SEQ ID NO: 2065), GAGguggguu (SEQ ID NO: 2066), GAGgugguau (SEQ ID NO: 2067), GAGgugguuc (SEQ ID NO: 2068), GAGgugucau (SEQ ID NO: 2069), GAGgugugag (SEQ ID NO: 2070), GAGgugugau (SEQ ID NO: 2071), GAGgugugca (SEQ ID NO: 2072), GAGgugugcu (SEQ ID NO: 2073), GAGgugugga (SEQ ID NO: 2074), GAGguguggg (SEQ ID NO: 2075), GAGguguggu (SEQ ID NO: 2076), GAGgugugua (SEQ ID NO: 2077), GAGgugugug (SEQ ID NO: 2078), GAGguuaaau (SEQ ID NO: 2079), GAGguuaaga (SEQ ID NO: 2080), GAGguuaaua (SEQ ID NO: 2081), GAGguuaccg (SEQ ID NO: 2082), GAGguuagaa (SEQ ID NO: 2083), GAGguuagac (SEQ ID NO: 2084), GAGguuagag (SEQ ID NO: 2085), GAGguuaggu (SEQ ID NO: 2086), GAGguuagua (SEQ ID NO: 2087), GAGguuaguc (SEQ ID NO: 2088), GAGguuagug (SEQ ID NO: 2089), GAGguuaguu (SEQ ID NO: 2090), GAGguuaugu (SEQ ID NO: 2091), GAGguuauuc (SEQ ID NO: 2092), GAGguucaaa (SEQ ID NO: 2093), GAGguucaua (SEQ ID NO: 2094), GAGguucuga (SEQ ID NO: 2095), GAGguugaag (SEQ ID NO: 2096), GAGguugcag (SEQ ID NO: 2097), GAGguugcug (SEQ ID NO: 2098), GAGguuggaa (SEQ ID NO: 2099), GAGguuggag (SEQ ID NO: 2100), GAGguuggau (SEQ ID NO: 2101), GAGguuggua (SEQ ID NO: 2102), GAGguugguc (SEQ ID NO: 2103), GAGguugguu (SEQ ID NO: 2104), GAGguuguag (SEQ ID NO: 2105), GAGguuucug (SEQ ID NO: 2106), GAGguuugag (SEQ ID NO: 2107), GAGguuugga (SEQ ID NO: 2108), GAGguuuggg (SEQ ID NO: 2109), GAGguuugua (SEQ ID NO: 2110), GAGguuuguu (SEQ ID NO: 2111), GAGguuuuca (SEQ ID NO: 2112), GAGguuuuga (SEQ ID NO: 2113), GAGguuuugg (SEQ ID NO: 2114), GAGguuuuua (SEQ ID NO: 2115), GAGguuuuuc (SEQ ID NO: 2116), GAUguaaaau (SEQ ID NO: 2117), GAUguaagca (SEQ ID NO: 2118), GAUguaagcc (SEQ ID NO: 2119), GAUguaaggu (SEQ ID NO: 2120), GAUguaagua (SEQ ID NO: 2121), GAUguaagug (SEQ ID NO: 2122), GAUguaaguu (SEQ ID NO: 2123), GAUguacauc (SEQ ID NO: 2124), GAUguaggua (SEQ ID NO: 2125), GAUguauggc (SEQ ID NO: 2126), GAUguaugua (SEQ ID NO: 2127), GAUguauguu (SEQ ID NO: 2128), GAUgucagug (SEQ ID NO: 2129), GAUgugagag (SEQ ID NO: 2130), GAUgugagcc (SEQ ID NO: 2131), GAUgugagcu (SEQ ID NO: 2132), GAUgugagga (SEQ ID NO: 2133), GAUgugaguc (SEQ ID NO: 2134), GAUgugagug (SEQ ID NO: 2135), GAUgugaguu (SEQ ID NO: 2136), GAUgugggua (SEQ ID NO: 2137), GAUgugggug (SEQ ID NO: 2138), GAUguguguu (SEQ ID NO: 2139), GAUguuagcu (SEQ ID NO: 2140), GAUguucagu (SEQ ID NO: 2141), GAUguucgug (SEQ ID NO: 2142), GAUguuuguu (SEQ ID NO: 2143), GCAguaaagg (SEQ ID NO: 2144), GCAguaagaa (SEQ ID NO: 2145), GCAguaagga (SEQ ID NO: 2146), GCAguaagua (SEQ ID NO: 2147), GCAguaaguc (SEQ ID NO: 2148), GCAguaaguu (SEQ ID NO: 2149), GCAguagaug (SEQ ID NO: 2150), GCAguaggua (SEQ ID NO: 2151), GCAguaugug (SEQ ID NO: 2152), GCAguauguu (SEQ ID NO: 2153), GCAgucagua (SEQ ID NO: 2154), GCAgucagug (SEQ ID NO: 2155), GCAguccggu (SEQ ID NO: 2156), GCAgugacuu (SEQ ID NO: 2157), GCAgugagcc (SEQ ID NO: 2158), GCAgugageg (SEQ ID NO: 2159), GCAgugagcu (SEQ ID NO: 2160), GCAgugagua (SEQ ID NO: 2161), GCAgugagug (SEQ ID NO: 2162), GCAgugaguu (SEQ ID NO: 2163), GCAgugggua (SEQ ID NO: 2164), GCAguuaagu (SEQ ID NO: 2165), GCAguugagu (SEQ ID NO: 2166), GCCguaaguc (SEQ ID NO: 2167), GCCgugagua (SEQ ID NO: 2168), GCGguaaagc (SEQ ID NO: 2169), GCGguaaaua (SEQ ID NO: 2170), GCGguaagcu (SEQ ID NO: 2171), GCGguaaggg (SEQ ID NO: 2172), GCGguaagug (SEQ ID NO: 2173), GCGguaauca (SEQ ID NO: 2174), GCGguacgua (SEQ ID NO: 2175), GCGguacuug (SEQ ID NO: 2176), GCGguagggu (SEQ ID NO: 2177), GCGguagugu (SEQ ID NO: 2178), GCGgugagca (SEQ ID NO: 2179), GCGgugagcu (SEQ ID NO: 2180), GCGgugaguu (SEQ ID NO: 2181), GCGguggcuc (SEQ ID NO: 2182), GCGgugugca (SEQ ID NO: 2183), GCGguguguu (SEQ ID NO: 2184), GCGguuaagu (SEQ ID NO: 2185), GCGguuugca (SEQ ID NO: 2186), GCUgcuguaa (SEQ ID NO: 2187), GCUguaaaua (SEQ ID NO: 2188), GCUguaagac (SEQ ID NO: 2189), GCUguaagag (SEQ ID NO: 2190), GCUguaagca (SEQ ID NO: 2191), GCUguaagga (SEQ ID NO: 2192), GCUguaagua (SEQ ID NO: 2193), GCUguaaguc (SEQ ID NO: 2194), GCUguaagug (SEQ ID NO: 2195), GCUguaaguu (SEQ ID NO: 2196), GCUguaggug (SEQ ID NO: 2197), GCUguauggu (SEQ ID NO: 2198), GCUgucagug (SEQ ID NO: 2199), GCUguccuug (SEQ ID NO: 2200), GCUgugagaa (SEQ ID NO: 2201), GCUgugagcc (SEQ ID NO: 2202), GCUgugagga (SEQ ID NO: 2203), GCUgugagua (SEQ ID NO: 2204), GCUgugaguc (SEQ ID NO: 2205), GCUgugagug (SEQ ID NO: 2206), GCUgugaguu (SEQ ID NO: 2207), GCUguggguu (SEQ ID NO: 2208), GGAguaagag (SEQ ID NO: 2209), GGAguaagca (SEQ ID NO: 2210), GGAguaagcc (SEQ ID NO: 2211), GGAguaagcu (SEQ ID NO: 2212), GGAguaagga (SEQ ID NO: 2213), GGAguaagug (SEQ ID NO: 2214), GGAguaaguu (SEQ ID NO: 2215), GGAguaauuu (SEQ ID NO: 2216), GGAguacugu (SEQ ID NO: 2217), GGAguaggaa (SEQ ID NO: 2218), GGAguaggua (SEQ ID NO: 2219), GGAguagguu (SEQ ID NO: 2220), GGAguaguau (SEQ ID NO: 2221), GGAguaugac (SEQ ID NO: 2222), GGAguauggu (SEQ ID NO: 2223), GGAgucaagu (SEQ ID NO: 2224), GGAgugaggg (SEQ ID NO: 2225), GGAgugagua (SEQ ID NO: 2226), GGAgugaguc (SEQ ID NO: 2227), GGAgugagug (SEQ ID NO: 2228), GGAgugaguu (SEQ ID NO: 2229), GGAgugcuuu (SEQ ID NO: 2230), GGAgugggca (SEQ ID NO: 2231), GGAgugggug (SEQ ID NO: 2232), GGAguuaagg (SEQ ID NO: 2233), GGAguugaga (SEQ ID NO: 2234), GGCguaagcc (SEQ ID NO: 2235), GGCguaggua (SEQ ID NO: 2236), GGCguaggug (SEQ ID NO: 2237), GGCgugagcc (SEQ ID NO: 2238), GGCgugaguc (SEQ ID NO: 2239), GGGguaaaca (SEQ ID NO: 2240), GGGguaaacc (SEQ ID NO: 2241), GGGguaaacu (SEQ ID NO: 2242), GGGguaagaa (SEQ ID NO: 2243), GGGguaagag (SEQ ID NO: 2244), GGGguaagau (SEQ ID NO: 2245), GGGguaagca (SEQ ID NO: 2246), GGGguaagcc (SEQ ID NO: 2247), GGGguaagcu (SEQ ID NO: 2248), GGGguaagga (SEQ ID NO: 2249), GGGguaaggg (SEQ ID NO: 2250), GGGguaagua (SEQ ID NO: 2251), GGGguaagug (SEQ ID NO: 2252), GGGguaaguu (SEQ ID NO: 2253), GGGguagaca (SEQ ID NO: 2254), GGGguaggag (SEQ ID NO: 2255), GGGguaggcc (SEQ ID NO: 2256), GGGguaggga (SEQ ID NO: 2257), GGGguaggua (SEQ ID NO: 2258), GGGguaggug (SEQ ID NO: 2259), GGGguagguu (SEQ ID NO: 2260), GGGguagugc (SEQ ID NO: 2261), GGGguaucug (SEQ ID NO: 2262), GGGguaugac (SEQ ID NO: 2263), GGGguaugga (SEQ ID NO: 2264), GGGguaugua (SEQ ID NO: 2265), GGGguauguc (SEQ ID NO: 2266), GGGguaugug (SEQ ID NO: 2267), GGGguauguu (SEQ ID NO: 2268), GGGgucagua (SEQ ID NO: 2269), GGGguccgug (SEQ ID NO: 2270), GGGgucggag (SEQ ID NO: 2271), GGGgucugug (SEQ ID NO: 2272), GGGgugaaca (SEQ ID NO: 2273), GGGgugaaga (SEQ ID NO: 2274), GGGgugagaa (SEQ ID NO: 2275), GGGgugagau (SEQ ID NO: 2276), GGGgugagcc (SEQ ID NO: 2277), GGGgugagcg (SEQ ID NO: 2278), GGGgugagcu (SEQ ID NO: 2279), GGGgugagga (SEQ ID NO: 2280), GGGgugaggc (SEQ ID NO: 2281), GGGgugaggg (SEQ ID NO: 2282), GGGgugaguc (SEQ ID NO: 2283), GGGgugagug (SEQ ID NO: 2284), GGGgugaguu (SEQ ID NO: 2285), GGGgugegua (SEQ ID NO: 2286), GGGguggggu (SEQ ID NO: 2287), GGGgugggua (SEQ ID NO: 2288), GGGgugggug (SEQ ID NO: 2289), GGGguggguu (SEQ ID NO: 2290), GGGgugugcg (SEQ ID NO: 2291), GGGgugugua (SEQ ID NO: 2292), GGGguguguc (SEQ ID NO: 2293), GGGgugugug (SEQ ID NO: 2294), GGGguuacag (SEQ ID NO: 2295), GGGguuggac (SEQ ID NO: 2296), GGGguuggga (SEQ ID NO: 2297), GGGguuugcc (SEQ ID NO: 2298), GGGguuugua (SEQ ID NO: 2299), GGUguaagaa (SEQ ID NO: 2300), GGUguaagau (SEQ ID NO: 2301), GGUguaagca (SEQ ID NO: 2302), GGUguaagcc (SEQ ID NO: 2303), GGUguaagcg (SEQ ID NO: 2304), GGUguaaguc (SEQ ID NO: 2305), GGUguaagug (SEQ ID NO: 2306), GGUguagguc (SEQ ID NO: 2307), GGUguaggug (SEQ ID NO: 2308), GGUguagguu (SEQ ID NO: 2309), GGUguccgua (SEQ ID NO: 2310), GGUgugagag (SEQ ID NO: 2311), GGUgugagcc (SEQ ID NO: 2312), GGUgugagcu (SEQ ID NO: 2313), GGUgugagua (SEQ ID NO: 2314), GGUgugaguc (SEQ ID NO: 2315), GGUgugcuuc (SEQ ID NO: 2316), GGUguggcug (SEQ ID NO: 2317), GGUgugguga (SEQ ID NO: 2318), GGUgugucug (SEQ ID NO: 2319), GGUguugaaa (SEQ ID NO: 2320), GGUguugcug (SEQ ID NO: 2321), GUAguaagau (SEQ ID NO: 2322), GUAguaagua (SEQ ID NO: 2323), GUAguaagug (SEQ ID NO: 2324), GUAguagcuu (SEQ ID NO: 2325), GUAguaggua (SEQ ID NO: 2326), GUAgucagua (SEQ ID NO: 2327), GUAgugagua (SEQ ID NO: 2328), GUAguggugg (SEQ ID NO: 2329), GUAguuaagu (SEQ ID NO: 2330), GUAguuucug (SEQ ID NO: 2331), GUCguaagug (SEQ ID NO: 2332), GUCgugagug (SEQ ID NO: 2333), GUCgugaguu (SEQ ID NO: 2334), GUGgcaagua (SEQ ID NO: 2335), GUGgcuugua (SEQ ID NO: 2336), GUGguaaaau (SEQ ID NO: 2337), GUGguaaaga (SEQ ID NO: 2338), GUGguaaauu (SEQ ID NO: 2339), GUGguaacau (SEQ ID NO: 2340), GUGguaacua (SEQ ID NO: 2341), GUGguaagaa (SEQ ID NO: 2342), GUGguaagac (SEQ ID NO: 2343), GUGguaagag (SEQ ID NO: 2344), GUGguaagau (SEQ ID NO: 2345), GUGguaagca (SEQ ID NO: 2346), GUGguaageg (SEQ ID NO: 2347), GUGguaagcu (SEQ ID NO: 2348), GUGguaagga (SEQ ID NO: 2349), GUGguaaggc (SEQ ID NO: 2350), GUGguaagua (SEQ ID NO: 2351), GUGguaaguc (SEQ ID NO: 2352), GUGguaagug (SEQ ID NO: 2353), GUGguaaguu (SEQ ID NO: 2354), GUGguaauga (SEQ ID NO: 2355), GUGguaauuc (SEQ ID NO: 2356), GUGguaauuu (SEQ ID NO: 2357), GUGguacaug (SEQ ID NO: 2358), GUGguacgau (SEQ ID NO: 2359), GUGguacuau (SEQ ID NO: 2360), GUGguacuug (SEQ ID NO: 2361), GUGguagaua (SEQ ID NO: 2362), GUGguagcgc (SEQ ID NO: 2363), GUGguaggga (SEQ ID NO: 2364), GUGguagguc (SEQ ID NO: 2365), GUGguaggug (SEQ ID NO: 2366), GUGguagguu (SEQ ID NO: 2367), GUGguauaaa (SEQ ID NO: 2368), GUGguaucuc (SEQ ID NO: 2369), GUGguaugaa (SEQ ID NO: 2370), GUGguaugau (SEQ ID NO: 2371), GUGguaugca (SEQ ID NO: 2372), GUGguaugua (SEQ ID NO: 2373), GUGguauguu (SEQ ID NO: 2374), GUGguccgug (SEQ ID NO: 2375), GUGgucuggc (SEQ ID NO: 2376), GUGgugaaac (SEQ ID NO: 2377), GUGgugagaa (SEQ ID NO: 2378), GUGgugagau (SEQ ID NO: 2379), GUGgugagca (SEQ ID NO: 2380), GUGgugagcu (SEQ ID NO: 2381), GUGgugagga (SEQ ID NO: 2382), GUGgugaggc (SEQ ID NO: 2383), GUGgugagug (SEQ ID NO: 2384), GUGgugaguu (SEQ ID NO: 2385), GUGgugauua (SEQ ID NO: 2386), GUGgugauuc (SEQ ID NO: 2387), GUGgugcgau (SEQ ID NO: 2388), GUGgugcuua (SEQ ID NO: 2389), GUGgugggaa (SEQ ID NO: 2390), GUGgugggua (SEQ ID NO: 2391), GUGguggguc (SEQ ID NO: 2392), GUGguguccg (SEQ ID NO: 2393), GUGguuagca (SEQ ID NO: 2394), GUGguuaggu (SEQ ID NO: 2395), GUGguuagug (SEQ ID NO: 2396), GUGguuugca (SEQ ID NO: 2397), GUGguuugua (SEQ ID NO: 2398), GUUguaaggu (SEQ ID NO: 2399), GUUguaagua (SEQ ID NO: 2400), GUUguaaguc (SEQ ID NO: 2401), GUUguaaguu (SEQ ID NO: 2402), GUUguaccac (SEQ ID NO: 2403), GUUguagcgu (SEQ ID NO: 2404), GUUguaugug (SEQ ID NO: 2405), GUUguauguu (SEQ ID NO: 2406), GUUgucugug (SEQ ID NO: 2407), GUUgugagcu (SEQ ID NO: 2408), GUUgugagug (SEQ ID NO: 2409), GUUgugaguu (SEQ ID NO: 2410), GUUgugggua (SEQ ID NO: 2411), GUUguggguu (SEQ ID NO: 2412), UAAguaaaug (SEQ ID NO: 2413), UAAguaacua (SEQ ID NO: 2414), UAAguaagaa (SEQ ID NO: 2415), UAAguaagag (SEQ ID NO: 2416), UAAguaagau (SEQ ID NO: 2417), UAAguaagca (SEQ ID NO: 2418), UAAguaagcu (SEQ ID NO: 2419), UAAguaagga (SEQ ID NO: 2420), UAAguaaggu (SEQ ID NO: 2421), UAAguaagua (SEQ ID NO: 2422), UAAguaaguc (SEQ ID NO: 2423), UAAguaagug (SEQ ID NO: 2424), UAAguaaguu (SEQ ID NO: 2425), UAAguaauaa (SEQ ID NO: 2426), UAAguacuag (SEQ ID NO: 2427), UAAguaguuu (SEQ ID NO: 2428), UAAguauaaa (SEQ ID NO: 2429), UAAguauaca (SEQ ID NO: 2430), UAAguaugua (SEQ ID NO: 2431), UAAguauuau (SEQ ID NO: 2432), UAAguauuuu (SEQ ID NO: 2433), UAAgucuuuu (SEQ ID NO: 2434), UAAgugagac (SEQ ID NO: 2435), UAAgugagga (SEQ ID NO: 2436), UAAgugaggg (SEQ ID NO: 2437), UAAgugagua (SEQ ID NO: 2438), UAAgugaguc (SEQ ID NO: 2439), UAAgugagug (SEQ ID NO: 2440), UAAgugaguu (SEQ ID NO: 2441), UAAgugaucc (SEQ ID NO: 2442), UAAgugauuc (SEQ ID NO: 2443), UAAgugcgug (SEQ ID NO: 2444), UAAguuaagu (SEQ ID NO: 2445), UAAguuccag (SEQ ID NO: 2446), UAAguucuuu (SEQ ID NO: 2447), UAAguuguaa (SEQ ID NO: 2448), UAAguuguau (SEQ ID NO: 2449), UAAguuuguu (SEQ ID NO: 2450), UACguaacug (SEQ ID NO: 2451), UACguaagaa (SEQ ID NO: 2452), UACguaagau (SEQ ID NO: 2453), UACguaagua (SEQ ID NO: 2454), UACguaagug (SEQ ID NO: 2455), UACguauccu (SEQ ID NO: 2456), UACgucuggc (SEQ ID NO: 2457), UACgugacca (SEQ ID NO: 2458), UAGgcaagac (SEQ ID NO: 2459), UAGgcaaguc (SEQ ID NO: 2460), UAGgcagguc (SEQ ID NO: 2461), UAGgcgugug (SEQ ID NO: 2462), UAGguaaaaa (SEQ ID NO: 2463), UAGguaaaac (SEQ ID NO: 2464), UAGguaaaag (SEQ ID NO: 2465), UAGguaaaau (SEQ ID NO: 2466), UAGguaaaca (SEQ ID NO: 2467), UAGguaaaga (SEQ ID NO: 2468), UAGguaaaua (SEQ ID NO: 2469), UAGguaaauc (SEQ ID NO: 2470), UAGguaaaug (SEQ ID NO: 2471), UAGguaaauu (SEQ ID NO: 2472), UAGguaacac (SEQ ID NO: 2473), UAGguaacag (SEQ ID NO: 2474), UAGguaacau (SEQ ID NO: 2475), UAGguaacca (SEQ ID NO: 2476), UAGguaacgg (SEQ ID NO: 2477), UAGguaacua (SEQ ID NO: 2478), UAGguaacuc (SEQ ID NO: 2479), UAGguaacug (SEQ ID NO: 2480), UAGguaacuu (SEQ ID NO: 2481), UAGguaagac (SEQ ID NO: 2482), UAGguaagag (SEQ ID NO: 2483), UAGguaagau (SEQ ID NO: 2484), UAGguaagca (SEQ ID NO: 2485), UAGguaagcc (SEQ ID NO: 2486), UAGguaagcu (SEQ ID NO: 2487), UAGguaagga (SEQ ID NO: 2488), UAGguaaggc (SEQ ID NO: 2489), UAGguaaggg (SEQ ID NO: 2490), UAGguaagua (SEQ ID NO: 2491), UAGguaaguc (SEQ ID NO: 2492), UAGguaagug (SEQ ID NO: 2493), UAGguaaguu (SEQ ID NO: 2494), UAGguaauag (SEQ ID NO: 2495), UAGguaauau (SEQ ID NO: 2496), UAGguaaucu (SEQ ID NO: 2497), UAGguaauga (SEQ ID NO: 2498), UAGguaaugg (SEQ ID NO: 2499), UAGguaaugu (SEQ ID NO: 2500), UAGguaauua (SEQ ID NO: 2501), UAGguaauuc (SEQ ID NO: 2502), UAGguaauuu (SEQ ID NO: 2503), UAGguacagc (SEQ ID NO: 2504), UAGguacagu (SEQ ID NO: 2505), UAGguacauu (SEQ ID NO: 2506), UAGguaccag (SEQ ID NO: 2507), UAGguaccua (SEQ ID NO: 2508), UAGguaccuu (SEQ ID NO: 2509), UAGguacgag (SEQ ID NO: 2510), UAGguacgua (SEQ ID NO: 2511), UAGguacguu (SEQ ID NO: 2512), UAGguacuau (SEQ ID NO: 2513), UAGguacuga (SEQ ID NO: 2514), UAGguacugg (SEQ ID NO: 2515), UAGguacuuc (SEQ ID NO: 2516), UAGguacuuu (SEQ ID NO: 2517), UAGguagcgg (SEQ ID NO: 2518), UAGguaggaa (SEQ ID NO: 2519), UAGguaggac (SEQ ID NO: 2520), UAGguaggau (SEQ ID NO: 2521), UAGguaggga (SEQ ID NO: 2522), UAGguagggg (SEQ ID NO: 2523), UAGguaggua (SEQ ID NO: 2524), UAGguagguc (SEQ ID NO: 2525), UAGguaggug (SEQ ID NO: 2526), UAGguagguu (SEQ ID NO: 2527), UAGguaguaa (SEQ ID NO: 2528), UAGguagucu (SEQ ID NO: 2529), UAGguagugg (SEQ ID NO: 2530), UAGguagugu (SEQ ID NO: 2531), UAGguaguuu (SEQ ID NO: 2532), UAGguauaaa (SEQ ID NO: 2533), UAGguauaac (SEQ ID NO: 2534), UAGguauaag (SEQ ID NO: 2535), UAGguauaau (SEQ ID NO: 2536), UAGguauaca (SEQ ID NO: 2537), UAGguauacu (SEQ ID NO: 2538), UAGguauaua (SEQ ID NO: 2539), UAGguauauc (SEQ ID NO: 2540), UAGguauauu (SEQ ID NO: 2541), UAGguaucag (SEQ ID NO: 2542), UAGguaucua (SEQ ID NO: 2543), UAGguaucuc (SEQ ID NO: 2544), UAGguaugaa (SEQ ID NO: 2545), UAGguaugag (SEQ ID NO: 2546), UAGguaugca (SEQ ID NO: 2547), UAGguaugga (SEQ ID NO: 2548), UAGguauggc (SEQ ID NO: 2549), UAGguauggu (SEQ ID NO: 2550), UAGguaugua (SEQ ID NO: 2551), UAGguauguc (SEQ ID NO: 2552), UAGguaugug (SEQ ID NO: 2553), UAGguauguu (SEQ ID NO: 2554), UAGguauuaa (SEQ ID NO: 2555), UAGguauuac (SEQ ID NO: 2556), UAGguauuau (SEQ ID NO: 2557), UAGguauuca (SEQ ID NO: 2558), UAGguauucc (SEQ ID NO: 2559), UAGguauucu (SEQ ID NO: 2560), UAGguauuga (SEQ ID NO: 2561), UAGguauuua (SEQ ID NO: 2562), UAGguauuuc (SEQ ID NO: 2563), UAGguauuuu (SEQ ID NO: 2564), UAGgucacuc (SEQ ID NO: 2565), UAGgucagcu (SEQ ID NO: 2566), UAGgucaggu (SEQ ID NO: 2567), UAGgucagua (SEQ ID NO: 2568), UAGgucagug (SEQ ID NO: 2569), UAGgucaguu (SEQ ID NO: 2570), UAGgucaucu (SEQ ID NO: 2571), UAGgucauug (SEQ ID NO: 2572), UAGguccaau (SEQ ID NO: 2573), UAGguccugu (SEQ ID NO: 2574), UAGgucucaa (SEQ ID NO: 2575), UAGgucucgc (SEQ ID NO: 2576), UAGgucuggc (SEQ ID NO: 2577), UAGgucuguc (SEQ ID NO: 2578), UAGgucugug (SEQ ID NO: 2579), UAGgugaagu (SEQ ID NO: 2580), UAGgugaaua (SEQ ID NO: 2581), UAGgugaaug (SEQ ID NO: 2582), UAGgugaauu (SEQ ID NO: 2583), UAGgugacau (SEQ ID NO: 2584), UAGgugacca (SEQ ID NO: 2585), UAGgugacua (SEQ ID NO: 2586), UAGgugagaa (SEQ ID NO: 2587), UAGgugagac (SEQ ID NO: 2588), UAGgugagag (SEQ ID NO: 2589), UAGgugagau (SEQ ID NO: 2590), UAGgugagcc (SEQ ID NO: 2591), UAGgugagcu (SEQ ID NO: 2592), UAGgugagga (SEQ ID NO: 2593), UAGgugaggc (SEQ ID NO: 2594), UAGgugaggu (SEQ ID NO: 2595), UAGgugagua (SEQ ID NO: 2596), UAGgugaguc (SEQ ID NO: 2597), UAGgugagug (SEQ ID NO: 2598), UAGgugauca (SEQ ID NO: 2599), UAGgugauuc (SEQ ID NO: 2600), UAGgugauuu (SEQ ID NO: 2601), UAGgugcaua (SEQ ID NO: 2602), UAGgugcauc (SEQ ID NO: 2603), UAGgugccgu (SEQ ID NO: 2604), UAGgugccug (SEQ ID NO: 2605), UAGgugcgca (SEQ ID NO: 2606), UAGgugcgua (SEQ ID NO: 2607), UAGgugcgug (SEQ ID NO: 2608), UAGgugcuga (SEQ ID NO: 2609), UAGguggaua (SEQ ID NO: 2610), UAGgugggaa (SEQ ID NO: 2611), UAGgugggac (SEQ ID NO: 2612), UAGgugggag (SEQ ID NO: 2613), UAGgugggau (SEQ ID NO: 2614), UAGgugggcc (SEQ ID NO: 2615), UAGgugggcu (SEQ ID NO: 2616), UAGguggguu (SEQ ID NO: 2617), UAGguggugu (SEQ ID NO: 2618), UAGguguaaa (SEQ ID NO: 2619), UAGgugugaa (SEQ ID NO: 2620), UAGgugugag (SEQ ID NO: 2621), UAGgugugca (SEQ ID NO: 2622), UAGgugugcc (SEQ ID NO: 2623), UAGgugugcg (SEQ ID NO: 2624), UAGguguggu (SEQ ID NO: 2625), UAGgugugua (SEQ ID NO: 2626), UAGgugugug (SEQ ID NO: 2627), UAGguguugg (SEQ ID NO: 2628), UAGguuaagc (SEQ ID NO: 2629), UAGguuagac (SEQ ID NO: 2630), UAGguuagcc (SEQ ID NO: 2631), UAGguuaggc (SEQ ID NO: 2632), UAGguuagua (SEQ ID NO: 2633), UAGguuaguc (SEQ ID NO: 2634), UAGguuagug (SEQ ID NO: 2635), UAGguucccc (SEQ ID NO: 2636), UAGguucuac (SEQ ID NO: 2637), UAGguuggua (SEQ ID NO: 2638), UAGguugguu (SEQ ID NO: 2639), UAGguugucc (SEQ ID NO: 2640), UAGguuuauu (SEQ ID NO: 2641), UAGguuugcc (SEQ ID NO: 2642), UAGguuugua (SEQ ID NO: 2643), UAGguuuguc (SEQ ID NO: 2644), UAGguuugug (SEQ ID NO: 2645), UAGguuuguu (SEQ ID NO: 2646), UAGguuuuuc (SEQ ID NO: 2647), UAGguuuuug (SEQ ID NO: 2648), UAUguaagaa (SEQ ID NO: 2649), UAUguaagau (SEQ ID NO: 2650), UAUguaagca (SEQ ID NO: 2651), UAUguaagcc (SEQ ID NO: 2652), UAUguaagua (SEQ ID NO: 2653), UAUguaaguc (SEQ ID NO: 2654), UAUguaagug (SEQ ID NO: 2655), UAUguaaguu (SEQ ID NO: 2656), UAUguacgug (SEQ ID NO: 2657), UAUguacguu (SEQ ID NO: 2658), UAUguagguc (SEQ ID NO: 2659), UAUguagguu (SEQ ID NO: 2660), UAUguauccu (SEQ ID NO: 2661), UAUguaucuc (SEQ ID NO: 2662), UAUguaugua (SEQ ID NO: 2663), UAUguauguc (SEQ ID NO: 2664), UAUguaugug (SEQ ID NO: 2665), UAUguauuau (SEQ ID NO: 2666), UAUgucagaa (SEQ ID NO: 2667), UAUgucugua (SEQ ID NO: 2668), UAUgugaaua (SEQ ID NO: 2669), UAUgugacag (SEQ ID NO: 2670), UAUgugagua (SEQ ID NO: 2671), UAUgugagug (SEQ ID NO: 2672), UAUgugaguu (SEQ ID NO: 2673), UAUgugggca (SEQ ID NO: 2674), UAUgugugua (SEQ ID NO: 2675), UAUguguuua (SEQ ID NO: 2676), UAUguuuugu (SEQ ID NO: 2677), UCAgcgacau (SEQ ID NO: 2678), UCAguaaaau (SEQ ID NO: 2679), UCAguaaaua (SEQ ID NO: 2680), UCAguaacug (SEQ ID NO: 2681), UCAguaagaa (SEQ ID NO: 2682), UCAguaagag (SEQ ID NO: 2683), UCAguaagau (SEQ ID NO: 2684), UCAguaagca (SEQ ID NO: 2685), UCAguaagcc (SEQ ID NO: 2686), UCAguaagcu (SEQ ID NO: 2687), UCAguaaggg (SEQ ID NO: 2688), UCAguaagua (SEQ ID NO: 2689), UCAguaaguc (SEQ ID NO: 2690), UCAguaagug (SEQ ID NO: 2691), UCAguaaguu (SEQ ID NO: 2692), UCAguaucuu (SEQ ID NO: 2693), UCAguaugga (SEQ ID NO: 2694), UCAguauggu (SEQ ID NO: 2695), UCAgucccca (SEQ ID NO: 2696), UCAgugagca (SEQ ID NO: 2697), UCAgugagcu (SEQ ID NO: 2698), UCAgugagua (SEQ ID NO: 2699), UCAgugagug (SEQ ID NO: 2700), UCAgugaguu (SEQ ID NO: 2701), UCAgugauug (SEQ ID NO: 2702), UCAgugggug (SEQ ID NO: 2703), UCAguugagc (SEQ ID NO: 2704), UCAguugauu (SEQ ID NO: 2705), UCAguuuagu (SEQ ID NO: 2706), UCCguaagca (SEQ ID NO: 2707), UCCguaagcu (SEQ ID NO: 2708), UCCguaaguc (SEQ ID NO: 2709), UCCguaagug (SEQ ID NO: 2710), UCCguaauag (SEQ ID NO: 2711), UCCguacuua (SEQ ID NO: 2712), UCCguaugua (SEQ ID NO: 2713), UCCguauguu (SEQ ID NO: 2714), UCCgugagau (SEQ ID NO: 2715), UCCgugaguc (SEQ ID NO: 2716), UCGguaaauu (SEQ ID NO: 2717), UCGguaagag (SEQ ID NO: 2718), UCGguaagcu (SEQ ID NO: 2719), UCGguacauc (SEQ ID NO: 2720), UCGguacucc (SEQ ID NO: 2721), UCGguagacc (SEQ ID NO: 2722), UCGguagguu (SEQ ID NO: 2723), UCGguaguaa (SEQ ID NO: 2724), UCGguaugug (SEQ ID NO: 2725), UCGguauguu (SEQ ID NO: 2726), UCGguauuga (SEQ ID NO: 2727), UCGgucagua (SEQ ID NO: 2728), UCGgucuuag (SEQ ID NO: 2729), UCGgugaagu (SEQ ID NO: 2730), UCGgugagaa (SEQ ID NO: 2731), UCGgugagca (SEQ ID NO: 2732), UCGgugaggc (SEQ ID NO: 2733), UCGgugagua (SEQ ID NO: 2734), UCGgugcgcu (SEQ ID NO: 2735), UCGgugcuuu (SEQ ID NO: 2736), UCGgugguuu (SEQ ID NO: 2737), UCGguuagcu (SEQ ID NO: 2738), UCUguaaaag (SEQ ID NO: 2739), UCUguaagaa (SEQ ID NO: 2740), UCUguaagau (SEQ ID NO: 2741), UCUguaagca (SEQ ID NO: 2742), UCUguaagcu (SEQ ID NO: 2743), UCUguaagua (SEQ ID NO: 2744), UCUguaaguc (SEQ ID NO: 2745), UCUguaagug (SEQ ID NO: 2746), UCUguaaguu (SEQ ID NO: 2747), UCUguaauaa (SEQ ID NO: 2748), UCUguaauga (SEQ ID NO: 2749), UCUguaaugu (SEQ ID NO: 2750), UCUguaggua (SEQ ID NO: 2751), UCUguagguu (SEQ ID NO: 2752), UCUguauaua (SEQ ID NO: 2753), UCUguaugac (SEQ ID NO: 2754), UCUguaugua (SEQ ID NO: 2755), UCUguccucg (SEQ ID NO: 2756), UCUgugagag (SEQ ID NO: 2757), UCUgugagcu (SEQ ID NO: 2758), UCUgugagga (SEQ ID NO: 2759), UCUgugagua (SEQ ID NO: 2760), UCUgugaguc (SEQ ID NO: 2761), UCUgugagug (SEQ ID NO: 2762), UCUgugaguu (SEQ ID NO: 2763), UCUgugcgua (SEQ ID NO: 2764), UCUgugugag (SEQ ID NO: 2765), UGAguaacuu (SEQ ID NO: 2766), UGAguaagau (SEQ ID NO: 2767), UGAguaagca (SEQ ID NO: 2768), UGAguaagcu (SEQ ID NO: 2769), UGAguaaggc (SEQ ID NO: 2770), UGAguaaggu (SEQ ID NO: 2771), UGAguaagua (SEQ ID NO: 2772), UGAguaaguc (SEQ ID NO: 2773), UGAguaagug (SEQ ID NO: 2774), UGAguaaguu (SEQ ID NO: 2775), UGAguaaucc (SEQ ID NO: 2776), UGAguaauua (SEQ ID NO: 2777), UGAguacagu (SEQ ID NO: 2778), UGAguacgua (SEQ ID NO: 2779), UGAguacguu (SEQ ID NO: 2780), UGAguacugu (SEQ ID NO: 2781), UGAguagcug (SEQ ID NO: 2782), UGAguaggua (SEQ ID NO: 2783), UGAguauaaa (SEQ ID NO: 2784), UGAguaugcu (SEQ ID NO: 2785), UGAguaugga (SEQ ID NO: 2786), UGAguaugua (SEQ ID NO: 2787), UGAguauguc (SEQ ID NO: 2788), UGAguauguu (SEQ ID NO: 2789), UGAgucagag (SEQ ID NO: 2790), UGAgucuacg (SEQ ID NO: 2791), UGAgugaaua (SEQ ID NO: 2792), UGAgugaauu (SEQ ID NO: 2793), UGAgugagaa (SEQ ID NO: 2794), UGAgugagau (SEQ ID NO: 2795), UGAgugagca (SEQ ID NO: 2796), UGAgugagce (SEQ ID NO: 2797), UGAgugagga (SEQ ID NO: 2798), UGAgugagua (SEQ ID NO: 2799), UGAgugagug (SEQ ID NO: 2800), UGAgugaguu (SEQ ID NO: 2801), UGAgugggaa (SEQ ID NO: 2802), UGAguuaaga (SEQ ID NO: 2803), UGAguuaaug (SEQ ID NO: 2804), UGAguuacgg (SEQ ID NO: 2805), UGAguuaggu (SEQ ID NO: 2806), UGAguucuau (SEQ ID NO: 2807), UGAguugguu (SEQ ID NO: 2808), UGAguuguag (SEQ ID NO: 2809), UGAguuuauc (SEQ ID NO: 2810), UGCguaaguc (SEQ ID NO: 2811), UGCguaagug (SEQ ID NO: 2812), UGCguacgge (SEQ ID NO: 2813), UGCguacggg (SEQ ID NO: 2814), UGCguaugua (SEQ ID NO: 2815), UGGgcaaguc (SEQ ID NO: 2816), UGGgcaagug (SEQ ID NO: 2817), UGGgcacauc (SEQ ID NO: 2818), UGGgccacgu (SEQ ID NO: 2819), UGGgeccegg (SEQ ID NO: 2820), UGGguaaaau (SEQ ID NO: 2821), UGGguaaage (SEQ ID NO: 2822), UGGguaaagg (SEQ ID NO: 2823), UGGguaaagu (SEQ ID NO: 2824), UGGguaaaua (SEQ ID NO: 2825), UGGguaaaug (SEQ ID NO: 2826), UGGguaaauu (SEQ ID NO: 2827), UGGguaacag (SEQ ID NO: 2828), UGGguaacau (SEQ ID NO: 2829), UGGguaacua (SEQ ID NO: 2830), UGGguaacuu (SEQ ID NO: 2831), UGGguagaa (SEQ ID NO: 2832), UGGguagac (SEQ ID NO: 2833), UGGguagag (SEQ ID NO: 2834), UGGguaagau (SEQ ID NO: 2835), UGGguaagca (SEQ ID NO: 2836), UGGguagee (SEQ ID NO: 2837), UGGguagcu (SEQ ID NO: 2838), UGGguaaggg (SEQ ID NO: 2839), UGGguaggu (SEQ ID NO: 2840), UGGguaagua (SEQ ID NO: 2841), UGGguaguc (SEQ ID NO: 2842), UGGguagug (SEQ ID NO: 2843), UGGguaaguu (SEQ ID NO: 2844), UGGguaaugu (SEQ ID NO: 2845), UGGguaauua (SEQ ID NO: 2846), UGGguaauuu (SEQ ID NO: 2847), UGGguacaaa (SEQ ID NO: 2848), UGGguacagu (SEQ ID NO: 2849), UGGguacuac (SEQ ID NO: 2850), UGGguaggga (SEQ ID NO: 2851), UGGguagguc (SEQ ID NO: 2852), UGGguaggug (SEQ ID NO: 2853), UGGguagguu (SEQ ID NO: 2854), UGGguaguua (SEQ ID NO: 2855), UGGguauagu (SEQ ID NO: 2856), UGGguaugaa (SEQ ID NO: 2857), UGGguaugac (SEQ ID NO: 2858), UGGguaugag (SEQ ID NO: 2859), UGGguaugua (SEQ ID NO: 2860), UGGguauguc (SEQ ID NO: 2861), UGGguaugug (SEQ ID NO: 2862), UGGguauguu (SEQ ID NO: 2863), UGGguauuug (SEQ ID NO: 2864), UGGgucuuug (SEQ ID NO: 2865), UGGgugaccu (SEQ ID NO: 2866), UGGgugacua (SEQ ID NO: 2867), UGGgugagac (SEQ ID NO: 2868), UGGgugagag (SEQ ID NO: 2869), UGGgugagca (SEQ ID NO: 2870), UGGgugagcc (SEQ ID NO: 2871), UGGgugagga (SEQ ID NO: 2872), UGGgugaggc (SEQ ID NO: 2873), UGGgugaggg (SEQ ID NO: 2874), UGGgugagua (SEQ ID NO: 2875), UGGgugaguc (SEQ ID NO: 2876), UGGgugagug (SEQ ID NO: 2877), UGGgugaguu (SEQ ID NO: 2878), UGGgugcgug (SEQ ID NO: 2879), UGGguggagg (SEQ ID NO: 2880), UGGguggcuu (SEQ ID NO: 2881), UGGguggggg (SEQ ID NO: 2882), UGGgugggua (SEQ ID NO: 2883), UGGguggguc (SEQ ID NO: 2884), UGGgugggug (SEQ ID NO: 2885), UGGguggguu (SEQ ID NO: 2886), UGGgugugga (SEQ ID NO: 2887), UGGguguguc (SEQ ID NO: 2888), UGGgugugug (SEQ ID NO: 2889), UGGguguguu (SEQ ID NO: 2890), UGGguguuua (SEQ ID NO: 2891), UGGguuaaug (SEQ ID NO: 2892), UGGguuaguc (SEQ ID NO: 2893), UGGguuagug (SEQ ID NO: 2894), UGGguuaguu (SEQ ID NO: 2895), UGGguucaag (SEQ ID NO: 2896), UGGguucgua (SEQ ID NO: 2897), UGGguuggug (SEQ ID NO: 2898), UGGguuuaag (SEQ ID NO: 2899), UGGguuugua (SEQ ID NO: 2900), UGUgcaagua (SEQ ID NO: 2901), UGUguaaaua (SEQ ID NO: 2902), UGUguaagaa (SEQ ID NO: 2903), UGUguaagac (SEQ ID NO: 2904), UGUguaagag (SEQ ID NO: 2905), UGUguaaggu (SEQ ID NO: 2906), UGUguaagua (SEQ ID NO: 2907), UGUguaaguc (SEQ ID NO: 2908), UGUguaaguu (SEQ ID NO: 2909), UGUguacuuc (SEQ ID NO: 2910), UGUguaggg (SEQ ID NO: 2911), UGUguaggua (SEQ ID NO: 2912), UGUguaguua (SEQ ID NO: 2913), UGUguaugug (SEQ ID NO: 2914), UGUgucagua (SEQ ID NO: 2915), UGUgucugua (SEQ ID NO: 2916), UGUgucuguc (SEQ ID NO: 2917), UGUgugaccc (SEQ ID NO: 2918), UGUgugagau (SEQ ID NO: 2919), UGUgugagca (SEQ ID NO: 2920), UGUgugagcc (SEQ ID NO: 2921), UGUgugagua (SEQ ID NO: 2922), UGUgugaguc (SEQ ID NO: 2923), UGUgugagug (SEQ ID NO: 2924), UGUgugcgug (SEQ ID NO: 2925), UGUgugggug (SEQ ID NO: 2926), UGUguggguu (SEQ ID NO: 2927), UGUgugugag (SEQ ID NO: 2928), UGUguguucu (SEQ ID NO: 2929), UGUguuuaga (SEQ ID NO: 2930), UUAguaaaua (SEQ ID NO: 2931), UUAguaagaa (SEQ ID NO: 2932), UUAguaagua (SEQ ID NO: 2933), UUAguaagug (SEQ ID NO: 2934), UUAguaaguu (SEQ ID NO: 2935), UUAguaggug (SEQ ID NO: 2936), UUAgugagca (SEQ ID NO: 2937), UUAgugaguu (SEQ ID NO: 2938), UUAguuaagu (SEQ ID NO: 2939), UUCguaaguc (SEQ ID NO: 2940), UUCguaaguu (SEQ ID NO: 2941), UUCguaauua (SEQ ID NO: 2942), UUCgugagua (SEQ ID NO: 2943), UUCgugaguu (SEQ ID NO: 2944), UUGgcaagug (SEQ ID NO: 2945), UUGgccgagu (SEQ ID NO: 2946), UUGguaaaaa (SEQ ID NO: 2947), UUGguaaaau (SEQ ID NO: 2948), UUGguaaaga (SEQ ID NO: 2949), UUGguaaagg (SEQ ID NO: 2950), UUGguaaagu (SEQ ID NO: 2951), UUGguaaauc (SEQ ID NO: 2952), UUGguaaaug (SEQ ID NO: 2953), UUGguaaauu (SEQ ID NO: 2954), UUGguaacug (SEQ ID NO: 2955), UUGguaacuu (SEQ ID NO: 2956), UUGguaagaa (SEQ ID NO: 2957), UUGguaagag (SEQ ID NO: 2958), UUGguaagcu (SEQ ID NO: 2959), UUGguaagga (SEQ ID NO: 2960), UUGguaaggg (SEQ ID NO: 2961), UUGguaagua (SEQ ID NO: 2962), UUGguaagug (SEQ ID NO: 2963), UUGguaaguu (SEQ ID NO: 2964), UUGguaauac (SEQ ID NO: 2965), UUGguaauca (SEQ ID NO: 2966), UUGguaaugc (SEQ ID NO: 2967), UUGguaaugu (SEQ ID NO: 2968), UUGguaauug (SEQ ID NO: 2969), UUGguaauuu (SEQ ID NO: 2970), UUGguacaua (SEQ ID NO: 2971), UUGguacgug (SEQ ID NO: 2972), UUGguagagg (SEQ ID NO: 2973), UUGguaggac (SEQ ID NO: 2974), UUGguaggcg (SEQ ID NO: 2975), UUGguaggcu (SEQ ID NO: 2976), UUGguaggga (SEQ ID NO: 2977), UUGguaggua (SEQ ID NO: 2978), UUGguagguc (SEQ ID NO: 2979), UUGguaggug (SEQ ID NO: 2980), UUGguauaaa (SEQ ID NO: 2981), UUGguauaca (SEQ ID NO: 2982), UUGguauauu (SEQ ID NO: 2983), UUGguaucua (SEQ ID NO: 2984), UUGguaucuc (SEQ ID NO: 2985), UUGguaugca (SEQ ID NO: 2986), UUGguaugua (SEQ ID NO: 2987), UUGguaugug (SEQ ID NO: 2988), UUGguauguu (SEQ ID NO: 2989), UUGguauugu (SEQ ID NO: 2990), UUGguauuua (SEQ ID NO: 2991), UUGguauuuu (SEQ ID NO: 2992), UUGgucagaa (SEQ ID NO: 2993), UUGgucagua (SEQ ID NO: 2994), UUGgucucug (SEQ ID NO: 2995), UUGgucugca (SEQ ID NO: 2996), UUGgugaaaa (SEQ ID NO: 2997), UUGgugacug (SEQ ID NO: 2998), UUGgugagac (SEQ ID NO: 2999), UUGgugagau (SEQ ID NO: 3000), UUGgugagca (SEQ ID NO: 3001), UUGgugagga (SEQ ID NO: 3002), UUGgugaggg (SEQ ID NO: 3003), UUGgugagua (SEQ ID NO: 3004), UUGgugaguc (SEQ ID NO: 3005), UUGgugagug (SEQ ID NO: 3006), UUGgugaguu (SEQ ID NO: 3007), UUGgugaugg (SEQ ID NO: 3008), UUGgugauua (SEQ ID NO: 3009), UUGgugauug (SEQ ID NO: 3010), UUGgugcaca (SEQ ID NO: 3011), UUGgugggaa (SEQ ID NO: 3012), UUGguggggc (SEQ ID NO: 3013), UUGgugggua (SEQ ID NO: 3014), UUGguggguc (SEQ ID NO: 3015), UUGgugggug (SEQ ID NO: 3016), UUGguggguu (SEQ ID NO: 3017), UUGguguggu (SEQ ID NO: 3018), UUGguguguc (SEQ ID NO: 3019), UUGgugugug (SEQ ID NO: 3020), UUGguguguu (SEQ ID NO: 3021), UUGguuaagu (SEQ ID NO: 3022), UUGguuagca (SEQ ID NO: 3023), UUGguuagug (SEQ ID NO: 3024), UUGguuaguu (SEQ ID NO: 3025), UUGguuggga (SEQ ID NO: 3026), UUGguugguu (SEQ ID NO: 3027), UUGguuugua (SEQ ID NO: 3028), UUGguuuguc (SEQ ID NO: 3029), UUUgcaagug (SEQ ID NO: 3030), UUUguaaaua (SEQ ID NO: 3031), UUUguaaaug (SEQ ID NO: 3032), UUUguaagaa (SEQ ID NO: 3033), UUUguaagac (SEQ ID NO: 3034), UUUguaagag (SEQ ID NO: 3035), UUUguaagca (SEQ ID NO: 3036), UUUguaaggu (SEQ ID NO: 3037), UUUguaagua (SEQ ID NO: 3038), UUUguaaguc (SEQ ID NO: 3039), UUUguaagug (SEQ ID NO: 3040), UUUguaaguu (SEQ ID NO: 3041), UUUguaauuu (SEQ ID NO: 3042), UUUguacagg (SEQ ID NO: 3043), UUUguacgug (SEQ ID NO: 3044), UUUguacuag (SEQ ID NO: 3045), UUUguacugu (SEQ ID NO: 3046), UUUguagguu (SEQ ID NO: 3047), UUUguauccu (SEQ ID NO: 3048), UUUguauguu (SEQ ID NO: 3049), UUUgugagca (SEQ ID NO: 3050), UUUgugagug (SEQ ID NO: 3051), UUUgugcguc (SEQ ID NO: 3052), UUUguguguc (SEQ ID NO: 3053), and uGGguaccug (SEQ ID NO: 3054).

Additional exemplary gene sequences and splice site sequences (e.g., 5′ splice site sequences) include AAGgcaagau (SEQ ID NO: 96), AUGguaugug (SEQ ID NO: 937), GGGgugaggc (SEQ ID NO: 2281), CAGguaggug (SEQ ID NO: 1222), AAGgucagua (SEQ ID NO: 293), AAGguuagag (SEQ ID NO: 3055), AUGgcacuua (SEQ ID NO: 3056), UAAguaaguc (SEQ ID NO: 2423), UGGgugagcu (SEQ ID NO: 3057), CGAgcugggc (SEQ ID NO: 3058), AAAgcacccc (SEQ ID NO: 3059), UAGguggggg (SEQ ID NO: 3060), AGAguaacgu (SEQ ID NO: 3061), UCGgugaugu (SEQ ID NO: 3062), AAUgucaguu (SEQ ID NO: 516), AGGgucugag (SEQ ID NO: 3063), GAGgugacug (SEQ ID NO: 3064), AUGguagguu (SEQ ID NO: 3065), GAGgucuguc (SEQ ID NO: 2000), CAGguaugug (SEQ ID NO: 1260), CAAguacugc (SEQ ID NO: 3066), CACgugcgua (SEQ ID NO: 3067), CCGgugagcu (SEQ ID NO: 3068), CAGguacuuc (SEQ ID NO: 3069), CAGgegagag (SEQ ID NO: 1115), GAAgcaagua (SEQ ID NO: 3070), AGGgugagca (SEQ ID NO: 789), CAGgcaaguc (SEQ ID NO: 3071), AAGgugaggc (SEQ ID NO: 344), CAGguaagua (SEQ ID NO: 1147), CCAguugggu (SEQ ID NO: 3072), AAGguguggg (SEQ ID NO: 3073), CAGguuggag (SEQ ID NO: 1484), CCGguaugaa (SEQ ID NO: 3074), UGGguaaugu (SEQ ID NO: 2845), CAGgugaggu (SEQ ID NO: 1344), AGAguaauag (SEQ ID NO: 3075), CAGguaugag (SEQ ID NO: 1249), AUGguaaguu (SEQ ID NO: 901), UUGguggguc (SEQ ID NO: 3015), UUUguaagca (SEQ ID NO: 3036), CUCguaugcc (SEQ ID NO: 3076), UAGguaagag (SEQ ID NO: 2483), UAGgcaaguu (SEQ ID NO: 3077), GGAguuaagu (SEQ ID NO: 3078), GAGguaugcc (SEQ ID NO: 1959), AAGguguggu (SEQ ID NO: 402), CAGgugggug (SEQ ID NO: 1415), UUAguaagua (SEQ ID NO: 2933), AAGguuggcu (SEQ ID NO: 3079), UGAguaugug (SEQ ID NO: 3080), CCAgccuucc (SEQ ID NO: 3081), CCUguacgug (SEQ ID NO: 3082), CCUguaggua (SEQ ID NO: 1601), CAGguacgcu (SEQ ID NO: 3083), GAGguucuuc (SEQ ID NO: 3084), AAGguugccu (SEQ ID NO: 3085), CGUguucacu (SEQ ID NO: 3086), CGGgugggga (SEQ ID NO: 3087), UAGgugggau (SEQ ID NO: 2614), CGGguaagga (SEQ ID NO: 3088), AAGguacuau (SEQ ID NO: 195), GGGguaagcu (SEQ ID NO: 2248), ACGguagagc (SEQ ID NO: 3089), CAGgugaaga (SEQ ID NO: 1318), GCGguaagag (SEQ ID NO: 3090), CAGguguugu (SEQ ID NO: 3091), GAAguuugug (SEQ ID NO: 3092), AUGgugagca (SEQ ID NO: 955), CGGguucgug (SEQ ID NO: 3093), AUUguccggc (SEQ ID NO: 3094), GAUgugugug (SEQ ID NO: 3095), AUGgucuguu (SEQ ID NO: 3096), AAGguaggau (SEQ ID NO: 219), CCGguaagau (SEQ ID NO: 1575), AAGguaaaga (SEQ ID NO: 126), GGGgugaguu (SEQ ID NO: 2285), AGGguuggug (SEQ ID NO: 808), GGAgugagug (SEQ ID NO: 2228), AGUguaagga (SEQ ID NO: 3097), UAGguaacug (SEQ ID NO: 2480), AAGgugaaga (SEQ ID NO: 3098), UGGguaagug (SEQ ID NO: 2843), CAGguaagag (SEQ ID NO: 1140), UAGgugagcg (SEQ ID NO: 3099), GAGguaaaaa (SEQ ID NO: 1865), GCCguaaguu (SEQ ID NO: 3100), AAGguuuugu (SEQ ID NO: 473), CAGgugagga (SEQ ID NO: 1341), ACAgcccaug (SEQ ID NO: 3101), GCGgugagcc (SEQ ID NO: 3102), CAGguaugca (SEQ ID NO: 1251), AUGguaccua (SEQ ID NO: 3103), CAAguaugua (SEQ ID NO: 1050), AUGguggugc (SEQ ID NO: 3104), UAAguggcag (SEQ ID NO: 3105), UAGguauagu (SEQ ID NO: 3106), CUGguauuua (SEQ ID NO: 3107), AGGguaaacg (SEQ ID NO: 3108), AUAguaagug (SEQ ID NO: 850), UUGguacuga (SEQ ID NO: 3109), GGUguaagcc (SEQ ID NO: 2303), GAGguggaua (SEQ ID NO: 3110), GAUguaagaa (SEQ ID NO: 3111), ACGgucaguu (SEQ ID NO: 3112), UAAguaaaca (SEQ ID NO: 3113), AAGguaucug (SEQ ID NO: 251), AGGguauuug (SEQ ID NO: 3114), AAGgugaaug (SEQ ID NO: 328), CUGgugaauu (SEQ ID NO: 1749), CAGguuuuuu (SEQ ID NO: 1514), CAUguaugug (SEQ ID NO: 1534), UUGguagagg (SEQ ID NO: 2973), AAGguaugcc (SEQ ID NO: 258), CAGgugccac (SEQ ID NO: 3115), UCGguauuga (SEQ ID NO: 2727), AAGguuugug (SEQ ID NO: 468), AAUguacagg (SEQ ID NO: 3116), CAUguggguu (SEQ ID NO: 1545), CAUgugaguu (SEQ ID NO: 1542), UUGguaaugu (SEQ ID NO: 2968), AGUguaggug (SEQ ID NO: 3117), GAGguaacuc (SEQ ID NO: 3118), GAGguggcgc (SEQ ID NO: 3119), CUGguaauug (SEQ ID NO: 3120), GAGguuugcu (SEQ ID NO: 3121), UGUguacgug (SEQ ID NO: 3122), UAGguaaaga (SEQ ID NO: 2468), CUAguaggca (SEQ ID NO: 3123), UCUgugaguc (SEQ ID NO: 2761), UCUguaaggc (SEQ ID NO: 3124), CAGguuugug (SEQ ID NO: 1509), GAGguagggc (SEQ ID NO: 1935), AAGguaacca (SEQ ID NO: 3125), ACUgugaguu (SEQ ID NO: 646), UAGguaauag (SEQ ID NO: 2495), AAAguaagcu (SEQ ID NO: 17), AUGgugagug (SEQ ID NO: 963), UAGguuugug (SEQ ID NO: 2645), AACguaggac (SEQ ID NO: 3126), GUAgcaggua (SEQ ID NO: 3127), GAGgucagac (SEQ ID NO: 3128), AGGguaugaa (SEQ ID NO: 3129), GAGguuagug (SEQ ID NO: 2089), CAGgcacgug (SEQ ID NO: 3130), GGGgcaagac (SEQ ID NO: 3131), CAGguguguc (SEQ ID NO: 1441), CAGguauuga (SEQ ID NO: 1265), CAGguauguc (SEQ ID NO: 1259), AAGgcaaggu (SEQ ID NO: 3132), UUGgugagaa (SEQ ID NO: 3133), AAGguaaaau (SEQ ID NO: 122), GGGguaagua (SEQ ID NO: 2251), AAGguaucuu (SEQ ID NO: 252), GACgugaguc (SEQ ID NO: 3134), UAUguaugcu (SEQ ID NO: 3135), AAGguacugu (SEQ ID NO: 199), CAGgugaacu (SEQ ID NO: 3136), CACguaaaug (SEQ ID NO: 3137), AAGgugugau (SEQ ID NO: 3138), GAAguauuug (SEQ ID NO: 3139), AAGgucugug (SEQ ID NO: 3140), AAGguggagg (SEQ ID NO: 3141), AAGguauaug (SEQ ID NO: 244), CAGguucuua (SEQ ID NO: 1477), AGGguaacca (SEQ ID NO: 730), CAGgugucac (SEQ ID NO: 1423), AAAguucugu (SEQ ID NO: 3142), UUGgugaguu (SEQ ID NO: 3007), CAAgugaguc (SEQ ID NO: 1067), UAGguagguc (SEQ ID NO: 2525), GCGgugagcu (SEQ ID NO: 2180), AUUgugagga (SEQ ID NO: 3143), CAGgugcaca (SEQ ID NO: 1361), CAGguuggaa (SEQ ID NO: 3144), CUGgucacuu (SEQ ID NO: 3145), GGAguaagug (SEQ ID NO: 2214), GAGgugggcu (SEQ ID NO: 2059), AAGguacuug (SEQ ID NO: 201), AGGguaggau (SEQ ID NO: 3146), AAUguguguu (SEQ ID NO: 3147), ACAguuaagu (SEQ ID NO: 568), GAGgugugug (SEQ ID NO: 2078), AAGgegggcu (SEQ ID NO: 3148), AUAgcaagua (SEQ ID NO: 3149), AAGguuguua (SEQ ID NO: 454), CAAgcaaggc (SEQ ID NO: 3150), GUGguaauua (SEQ ID NO: 3151), UCUguucagu (SEQ ID NO: 3152), AGGguaggcc (SEQ ID NO: 754), AAGguaucau (SEQ ID NO: 3153), UAGguaccuu (SEQ ID NO: 2509), AAGguaugac (SEQ ID NO: 254), GGAguaggua (SEQ ID NO: 2219), UAAguuggca (SEQ ID NO: 3154), AGUgugaggc (SEQ ID NO: 3155), GAGguuugug (SEQ ID NO: 3156), UGGgucugcu (SEQ ID NO: 3157), CAGgugaucc (SEQ ID NO: 1350), CAGgucagug (SEQ ID NO: 1283), AAGguaaggg (SEQ ID NO: 151), CAGgugcagu (SEQ ID NO: 3158), GAGguggguc (SEQ ID NO: 2064), GCUgugagug (SEQ ID NO: 2206), AAGguggagu (SEQ ID NO: 3159), GGGgucaguu (SEQ ID NO: 3160), AGCguaagug (SEQ ID NO: 719), AGAguaugaa (SEQ ID NO: 691), GGGguagggu (SEQ ID NO: 3161), AAGgccagca (SEQ ID NO: 3162), CGAguaugcc (SEQ ID NO: 3163), GUGgugagcg (SEQ ID NO: 3164), AAUguaaauu (SEQ ID NO: 481), CAGgugcgca (SEQ ID NO: 1375), GGUguaugaa (SEQ ID NO: 3165), CUUgugaguu (SEQ ID NO: 1804), AAGguaucuc (SEQ ID NO: 250), AGAguaagga (SEQ ID NO: 665), UAGguaagac (SEQ ID NO: 2482), GAGgugagug (SEQ ID NO: 2026), CAGguguguu (SEQ ID NO: 1443), UUGgugagua (SEQ ID NO: 3004), AGGgcgaguu (SEQ ID NO: 3166), CAGguuuugc (SEQ ID NO: 3167), UUUgugaguu (SEQ ID NO: 3168), AGGguaagca (SEQ ID NO: 736), GAGguccucu (SEQ ID NO: 3169), CCAgcaggua (SEQ ID NO: 3170), GAGguucgcg (SEQ ID NO: 3171), CAGgugaucu (SEQ ID NO: 1351), ACUguaagua (SEQ ID NO: 625), AAGguaaauc (SEQ ID NO: 131), CAGgcaaaua (SEQ ID NO: 3172), GUGguaagca (SEQ ID NO: 2346), CAGguuaaau (SEQ ID NO: 3173), UUGguaauaa (SEQ ID NO: 3174), UAUguaggua (SEQ ID NO: 3175), CAGguaguau (SEQ ID NO: 1225), AAGgugugcc (SEQ ID NO: 3176), UGGguaagag (SEQ ID NO: 2834), CAGgcaagca (SEQ ID NO: 3177), UUGguaaggg (SEQ ID NO: 2961), AAGgcaggug (SEQ ID NO: 109), ACGguaaaug (SEQ ID NO: 3178), GCUgugagca (SEQ ID NO: 3179), AUGguacaca (SEQ ID NO: 3180), GUAguguguu (SEQ ID NO: 3181), ACUguaagag (SEQ ID NO: 3182), CCCgcagguc (SEQ ID NO: 3183), GAGgugagcc (SEQ ID NO: 2019), GAGgugcugu (SEQ ID NO: 3184), UAAguaugcu (SEQ ID NO: 3185), GAGgccaucu (SEQ ID NO: 3186), UCAgugagug (SEQ ID NO: 2700), CAGgugcuac (SEQ ID NO: 3187), AAUgugggug (SEQ ID NO: 533), GAGgugugaa (SEQ ID NO: 3188), CUGguagguc (SEQ ID NO: 1730), GUGgcgcgeg (SEQ ID NO: 3189), CAGgugcaaa (SEQ ID NO: 1359), UAAguggagg (SEQ ID NO: 3190), CAUgugggua (SEQ ID NO: 3191), GAGguagggu (SEQ ID NO: 3192), AAAgugaguu (SEQ ID NO: 61), AGGguucuag (SEQ ID NO: 3193), UGUgugagcu (SEQ ID NO: 3194), AGGgugaauc (SEQ ID NO: 3195), CAGgucaggg (SEQ ID NO: 3196), AAGgucccug (SEQ ID NO: 3197), CUGguagagu (SEQ ID NO: 3198), UAGgucaguu (SEQ ID NO: 2570), AAAguaaggg (SEQ ID NO: 19), CAAguaugug (SEQ ID NO: 1052), CAGgugcuuu (SEQ ID NO: 3199), AAGguaauuc (SEQ ID NO: 169), GGGgugcacg (SEQ ID NO: 3200), ACUgugcuac (SEQ ID NO: 3201), CAGguaccua (SEQ ID NO: 3202), CAGguagcuu (SEQ ID NO: 1211), UGGgugaggc (SEQ ID NO: 2873), CUGguacauu (SEQ ID NO: 1718), AGGguaaucu (SEQ ID NO: 3203), CAGguacaag (SEQ ID NO: 1161), CAGguaauuc (SEQ ID NO: 1157), AGGgcacuug (SEQ ID NO: 3204), UAGgugagaa (SEQ ID NO: 2587), GAGguaaugc (SEQ ID NO: 3205), CCAgugaguu (SEQ ID NO: 3206), AAAguaugug (SEQ ID NO: 44), CUGgugaauc (SEQ ID NO: 3207), UAUguaugua (SEQ ID NO: 2663), CCUgcaggug (SEQ ID NO: 3208), CAGguaucug (SEQ ID NO: 1245), GAGgugaggu (SEQ ID NO: 3209), CUGguaaaac (SEQ ID NO: 3210), UGUgugugcu (SEQ ID NO: 3211), CAGguuaagu (SEQ ID NO: 3212), CAGguaaucc (SEQ ID NO: 1152), UAGguauuug (SEQ ID NO: 3213), UGGguagguc (SEQ ID NO: 2852), CAGguaacag (SEQ ID NO: 1129), AGCgugcgug (SEQ ID NO: 3214), AAGgucagga (SEQ ID NO: 289), GGUgugagcc (SEQ ID NO: 2312), CUGguaagua (SEQ ID NO: 1707), GGGgugggca (SEQ ID NO: 3215), AAGgugggaa (SEQ ID NO: 376), CAGgugagug (SEQ ID NO: 1347), CUGguuguua (SEQ ID NO: 3216), CAGguaauag (SEQ ID NO: 3217), UAGgugaguu (SEQ ID NO: 3218), AGAguaaguu (SEQ ID NO: 671), UAGguaaucc (SEQ ID NO: 3219), CCGgugacug (SEQ ID NO: 3220), GUCgugauua (SEQ ID NO: 3221), CUUguaagug (SEQ ID NO: 1794), UAGguaguca (SEQ ID NO: 3222), CUGguaaguc (SEQ ID NO: 3223), AGGgugageg (SEQ ID NO: 3224), CAGguaugga (SEQ ID NO: 1255), AUUgugacca (SEQ ID NO: 3225), GUUgugggua (SEQ ID NO: 2411), AAGguacaag (SEQ ID NO: 173), CUAgcaagug (SEQ ID NO: 3226), CUGgugagau (SEQ ID NO: 3227), CAGgugggca (SEQ ID NO: 1406), AUGgcucgag (SEQ ID NO: 3228), CUGguacguu (SEQ ID NO: 1720), UUGgugugua (SEQ ID NO: 3229), GAGgugucug (SEQ ID NO: 3230), GAGgugggac (SEQ ID NO: 3231), GGGgugggag (SEQ ID NO: 3232), GCAgcgugag (SEQ ID NO: 3233), GAGguaaaga (SEQ ID NO: 1870), GAGguaugua (SEQ ID NO: 1965), AAGgugagac (SEQ ID NO: 336), AAGguacaau (SEQ ID NO: 174), CUGguaugag (SEQ ID NO: 3234), AACguaaaau (SEQ ID NO: 3235), GUGguaggga (SEQ ID NO: 2364), CUGguaugug (SEQ ID NO: 1737), CUUguaagca (SEQ ID NO: 3236), AAGguaggga (SEQ ID NO: 223), AUUguaagcc (SEQ ID NO: 3237), AUGguaagcu (SEQ ID NO: 895), CAGgugaauu (SEQ ID NO: 1322), UAGgugaaua (SEQ ID NO: 2581), CAAguaugga (SEQ ID NO: 3238), AUGguauggc (SEQ ID NO: 936), GAGgucaugc (SEQ ID NO: 3239), CAGguacccu (SEQ ID NO: 1174), ACAgugagac (SEQ ID NO: 3240), CAGgucugau (SEQ ID NO: 3241), GAAguugggu (SEQ ID NO: 3242), CUGgugcgug (SEQ ID NO: 1767), CAGguacgag (SEQ ID NO: 1180), ACAgugagcc (SEQ ID NO: 556), AAGguaagua (SEQ ID NO: 153), GGAguaaggc (SEQ ID NO: 3243), GAGgugugua (SEQ ID NO: 2077), AAGgucauuu (SEQ ID NO: 3244), CAGguagucu (SEQ ID NO: 3245), AUGguaucug (SEQ ID NO: 3246), AAGguaaacu (SEQ ID NO: 125), GAGguaggug (SEQ ID NO: 1938), CUGguaagca (SEQ ID NO: 1700), AGGguaagag (SEQ ID NO: 734), AAAguaaagc (SEQ ID NO: 3247), CAGguuugag (SEQ ID NO: 1502), GAGgcgggua (SEQ ID NO: 3248), CGAguacgau (SEQ ID NO: 3249), CAGguuguug (SEQ ID NO: 1495), AAAguauggg (SEQ ID NO: 3250), UAGgcugguc (SEQ ID NO: 3251), AAGguaagga (SEQ ID NO: 149), AAGguuuccu (SEQ ID NO: 458), UUGguaaaac (SEQ ID NO: 3252), GAGguaagua (SEQ ID NO: 1893), CAGguucaag (SEQ ID NO: 1465), UGGguuaugu (SEQ ID NO: 3253), GAGgugaguu (SEQ ID NO: 2027), ACGgugaaac (SEQ ID NO: 598), GAUguaacca (SEQ ID NO: 3254), AAGgugcggg (SEQ ID NO: 3255), CCGguacgug (SEQ ID NO: 3256), GAUgugagaa (SEQ ID NO: 3257), GUGgcgguga (SEQ ID NO: 3258), CAGguauuag (SEQ ID NO: 3259), GAGguuggga (SEQ ID NO: 3260), AAGgcuagua (SEQ ID NO: 3261), AAGgugggcg (SEQ ID NO: 381), CAGgcaggga (SEQ ID NO: 3262), AAUguuaguu (SEQ ID NO: 3263), GAGguaaagg (SEQ ID NO: 3264), CAGgugugcu (SEQ ID NO: 1437), CUGguaugau (SEQ ID NO: 1733), AUGguuaguc (SEQ ID NO: 978), CUGgugagaa (SEQ ID NO: 1751), CAGgccggcg (SEQ ID NO: 3265), CAGgugacug (SEQ ID NO: 1332), AAAguaaggu (SEQ ID NO: 20), UAAguacuug (SEQ ID NO: 3266), AAGguaaagc (SEQ ID NO: 127), UCGguagggg (SEQ ID NO: 3267), CAGguaggaa (SEQ ID NO: 1212), AGUguaagca (SEQ ID NO: 817), CCCgugagau (SEQ ID NO: 3268), GUGguuguuu (SEQ ID NO: 3269), CAGguuugcc (SEQ ID NO: 1504), AGGguauggg (SEQ ID NO: 766), UAAguaagug (SEQ ID NO: 2424), GAGguaagac (SEQ ID NO: 3270), GAUguagguc (SEQ ID NO: 3271), CAAguaggug (SEQ ID NO: 1043), AUAguaaaua (SEQ ID NO: 845), GAGguugggg (SEQ ID NO: 3272), GAGgcgagua (SEQ ID NO: 3273), CAGguagugu (SEQ ID NO: 1229), GUGguaggug (SEQ ID NO: 2366), CAAgugagug (SEQ ID NO: 1068), AAGgugacaa (SEQ ID NO: 330), CCAgcguaau (SEQ ID NO: 3274), ACGgugaggu (SEQ ID NO: 3275), GGGguauauu (SEQ ID NO: 3276), CAGgugagua (SEQ ID NO: 1345), AAGgugegug (SEQ ID NO: 364), UAUguaaauu (SEQ ID NO: 3277), CAGgucagua (SEQ ID NO: 1281), ACGguacuua (SEQ ID NO: 3278), GAGgucagca (SEQ ID NO: 3279), UAAguaugua (SEQ ID NO: 2431), GGGgucagac (SEQ ID NO: 3280), AAUgugugag (SEQ ID NO: 3281), UCCgucagua (SEQ ID NO: 3282), CAGgugcuuc (SEQ ID NO: 1391), CCAguuagug (SEQ ID NO: 3283), CCGgugggg (SEQ ID NO: 1590), AGGgugcaug (SEQ ID NO: 3284), GGGguaggau (SEQ ID NO: 3285), UAGgugggcc (SEQ ID NO: 2615), GAGguguucg (SEQ ID NO: 3286), UUGgcaagaa (SEQ ID NO: 3287), UCCguaagua (SEQ ID NO: 3288), CAGguguaag (SEQ ID NO: 3289), CUCgugagua (SEQ ID NO: 1680), GAGguguuuu (SEQ ID NO: 3290), GAGgugagca (SEQ ID NO: 2018), GAGguaaagu (SEQ ID NO: 1872), AAGguacguu (SEQ ID NO: 193), CAGguccagu (SEQ ID NO: 1291), AUGgugaaac (SEQ ID NO: 947), GUAgugagcu (SEQ ID NO: 3291), CAGgugaaaa (SEQ ID NO: 3292), AGGguacagg (SEQ ID NO: 3293), AAGguaacgc (SEQ ID NO: 3294), AAGguauacc (SEQ ID NO: 3295), CCUgugagau (SEQ ID NO: 3296), GGGguacgug (SEQ ID NO: 3297), GAGguauggu (SEQ ID NO: 1964), UAGguauuau (SEQ ID NO: 2557), GAAguaggag (SEQ ID NO: 3298), UCGguaaggg (SEQ ID NO: 3299), CCGguaagcg (SEQ ID NO: 3300), GAAguaauua (SEQ ID NO: 1823), CAGgugaguc (SEQ ID NO: 1346), AAGgucaaga (SEQ ID NO: 279), AUGguaaguc (SEQ ID NO: 899), CAGgugagcu (SEQ ID NO: 1340), CCAguuuuug (SEQ ID NO: 3301), CAGgugggag (SEQ ID NO: 1404), AAGguauuau (SEQ ID NO: 270), AAGguaaaua (SEQ ID NO: 130), AAGgugcugu (SEQ ID NO: 3302), AAAguacacc (SEQ ID NO: 3303), CUGguucgug (SEQ ID NO: 1783), UCAguaaguc (SEQ ID NO: 2690), GAAguacgug (SEQ ID NO: 3304), CAGgugacaa (SEQ ID NO: 1323), UGGguaagaa (SEQ ID NO: 2832), UGUguagggg (SEQ ID NO: 3305), GAGguaggca (SEQ ID NO: 1932), UUGgugaggc (SEQ ID NO: 3306), AUGgugugua (SEQ ID NO: 974), CAGguccucc (SEQ ID NO: 3307), UUGguaaaug (SEQ ID NO: 2953), GCUgugaguu (SEQ ID NO: 2207), AUGgucugua (SEQ ID NO: 3308), CAUgcaggug (SEQ ID NO: 3309), CUGguacacc (SEQ ID NO: 3310), CAGguccuua (SEQ ID NO: 3311), CAAguaaucu (SEQ ID NO: 1031), AUGgcagccu (SEQ ID NO: 3312), AAGgucagaa (SEQ ID NO: 282), AACgugaggc (SEQ ID NO: 3313), CAGgcacgca (SEQ ID NO: 1106), ACGguccagg (SEQ ID NO: 3314), UCUguacaua (SEQ ID NO: 3315), GAGgugauua (SEQ ID NO: 3316), ACGguaaaua (SEQ ID NO: 3317), AUGguaacug (SEQ ID NO: 3318), CAGgcgcguu (SEQ ID NO: 3319), CAGguauaga (SEQ ID NO: 1235), AAGguuuguu (SEQ ID NO: 3320), CAGguaugaa (SEQ ID NO: 1247), UAGguuggua (SEQ ID NO: 2638), CUGgugagac (SEQ ID NO: 1752), CAGguuagga (SEQ ID NO: 3321), AUGgugacug (SEQ ID NO: 3322), UUGguauccc (SEQ ID NO: 3323), CUUguaggac (SEQ ID NO: 3324), AAAguguguu (SEQ ID NO: 69), CAGguuucuu (SEQ ID NO: 1500), GGGguauggc (SEQ ID NO: 3325), GGGguaggac (SEQ ID NO: 3326), ACUguaaguc (SEQ ID NO: 626), AUCguaagcu (SEQ ID NO: 3327), UAGguucccc (SEQ ID NO: 2636), GGUgugagca (SEQ ID NO: 3328), CUGguuggua (SEQ ID NO: 3329), GGGguuaggg (SEQ ID NO: 3330), UGAguaagaa (SEQ ID NO: 3331), GAGguauucc (SEQ ID NO: 1969), UGGguuaguc (SEQ ID NO: 2893), CAGgcucgug (SEQ ID NO: 3332), UAGguagagu (SEQ ID NO: 3333), UAGgugcccu (SEQ ID NO: 3334), AAAgugagua (SEQ ID NO: 58), GAGguucaua (SEQ ID NO: 2094), UUGguaagag (SEQ ID NO: 2958), ACCgugugua (SEQ ID NO: 3335), UAUguaguau (SEQ ID NO: 3336), UGGguaauag (SEQ ID NO: 3337), CAGgucugaa (SEQ ID NO: 3338), AAAguauaaa (SEQ ID NO: 3339), GUGgugaguc (SEQ ID NO: 3340), AGUgugauua (SEQ ID NO: 3341), UUGgugugug (SEQ ID NO: 3020), CAGgugaugg (SEQ ID NO: 1353), GCUgugagua (SEQ ID NO: 2204), CAGguacaug (SEQ ID NO: 1169), AAGguacagu (SEQ ID NO: 178), GAAguuguag (SEQ ID NO: 3342), CAGgugauua (SEQ ID NO: 1355), UAGgugaauu (SEQ ID NO: 2583), GGUguuaaua (SEQ ID NO: 3343), CAGguauuua (SEQ ID NO: 1268), CAAguacucg (SEQ ID NO: 3344), CAAguaagaa (SEQ ID NO: 1022), AAGguaccuu (SEQ ID NO: 188), ACGgugaggg (SEQ ID NO: 3345), UGAgcaggca (SEQ ID NO: 3346), GGGgugaccg (SEQ ID NO: 3347), GAGguaaaug (SEQ ID NO: 1875), CGGguuugug (SEQ ID NO: 3348), AAGgugageg (SEQ ID NO: 341), GUGguaugga (SEQ ID NO: 3349), CUGguaagga (SEQ ID NO: 1703), GAGguaccag (SEQ ID NO: 1911), CCGgugagug (SEQ ID NO: 1587), AAGguuagaa (SEQ ID NO: 416), GAGguacuug (SEQ ID NO: 1921), AGAguaaaac (SEQ ID NO: 651), UCUgugagua (SEQ ID NO: 2760), AAGgeggga (SEQ ID NO: 3350), CAGguaugcg (SEQ ID NO: 1253), AGGguaaaac (SEQ ID NO: 3351), AAGgugacug (SEQ ID NO: 333), AGGguauguu (SEQ ID NO: 3352), AAGguaugua (SEQ ID NO: 263), CAGgucucuc (SEQ ID NO: 1302), CAGgcaugua (SEQ ID NO: 3353), CUGguaggua (SEQ ID NO: 1729), AAGgucaugc (SEQ ID NO: 3354), CAGguacaca (SEQ ID NO: 1163), GAUguacguu (SEQ ID NO: 3355), ACAguacgug (SEQ ID NO: 3356), ACGguaccca (SEQ ID NO: 3357), CAGguagugc (SEQ ID NO: 3358), ACAguaagag (SEQ ID NO: 3359), GGUgcacacc (SEQ ID NO: 3360), GAGguguaac (SEQ ID NO: 3361), AAGgugugua (SEQ ID NO: 403), UAGguacuua (SEQ ID NO: 3362), GCGguacugc (SEQ ID NO: 3363), UGGguaaguc (SEQ ID NO: 2842), CAUguaggua (SEQ ID NO: 1529), CAGguaggau (SEQ ID NO: 3364), CAGgucuggc (SEQ ID NO: 3365), GUGguuuuaa (SEQ ID NO: 3366), CAGgugggaa (SEQ ID NO: 1402), UGGgugagua (SEQ ID NO: 2875), CGAgugagcc (SEQ ID NO: 3367), AAGguauggc (SEQ ID NO: 261), AGUguuguca (SEQ ID NO: 3368), CAGgugauuu (SEQ ID NO: 1358), UAGguaucuc (SEQ ID NO: 2544), UAAguauguu (SEQ ID NO: 3369), AAGguugagc (SEQ ID NO: 3370), AGAguaaaga (SEQ ID NO: 653), GGUguaagua (SEQ ID NO: 3371), GGGgugagcu (SEQ ID NO: 2279), CAGguauaau (SEQ ID NO: 3372), GAGguacaaa (SEQ ID NO: 1904), AUGguaccaa (SEQ ID NO: 3373), UAGguagggg (SEQ ID NO: 2523), UGAgucagaa (SEQ ID NO: 3374), AAGgcaauua (SEQ ID NO: 3375), UUGguaagau (SEQ ID NO: 3376), CAGguacaga (SEQ ID NO: 1165), AGAguuagag (SEQ ID NO: 3377), CAGgugeguc (SEQ ID NO: 1381), GAGguauuac (SEQ ID NO: 3378), ACGguacaga (SEQ ID NO: 3379), CAGgucuucc (SEQ ID NO: 1313), AAGguaaggu (SEQ ID NO: 152), GAGguaauuu (SEQ ID NO: 1903), AGUguaggcu (SEQ ID NO: 3380), AAAguaagcg (SEQ ID NO: 3381), CCUguaagcc (SEQ ID NO: 3382), AGGgugauuu (SEQ ID NO: 3383), UGUguaugaa (SEQ ID NO: 3384), CUGguacaca (SEQ ID NO: 3385), AGGguagaga (SEQ ID NO: 3386), AUAguaagca (SEQ ID NO: 848), AGAguaugua (SEQ ID NO: 3387), UUGgucagca (SEQ ID NO: 3388), CAGgcaaguu (SEQ ID NO: 1105), AAGguauaua (SEQ ID NO: 242), AAGgucugga (SEQ ID NO: 314), CAGguacgca (SEQ ID NO: 1181), AGGgugcggg (SEQ ID NO: 3389), AUGguaagug (SEQ ID NO: 900), AAAgugauga (SEQ ID NO: 3390), UGCgugagua (SEQ ID NO: 3391), AGAguaggga (SEQ ID NO: 684), UGUguaggua (SEQ ID NO: 2912), UAGguaggau (SEQ ID NO: 2521), UAAgugagug (SEQ ID NO: 2440), GCUguaagua (SEQ ID NO: 2193), GAAguaagaa (SEQ ID NO: 1814), UCGgugaggc (SEQ ID NO: 2733), UAGguauuuu (SEQ ID NO: 2564), AAGguacaca (SEQ ID NO: 3392), AAGguaggua (SEQ ID NO: 227), UGGguagguu (SEQ ID NO: 2854), ACAgcaagua (SEQ ID NO: 541), GAGguaggag (SEQ ID NO: 1931), UGGgugaguu (SEQ ID NO: 2878), GCGgugagau (SEQ ID NO: 3393), CCUguagguu (SEQ ID NO: 3394), CAGgugugua (SEQ ID NO: 1440), CUGguaagcc (SEQ ID NO: 1701), AAGgugauuc (SEQ ID NO: 3395), CAGguagcua (SEQ ID NO: 1208), GUUguaagug (SEQ ID NO: 3396), AUGguaagca (SEQ ID NO: 893), AUAguaggga (SEQ ID NO: 3397), GGGguucgcu (SEQ ID NO: 3398), CCGgucagag (SEQ ID NO: 3399), GUAguaugag (SEQ ID NO: 3400), CGUguaagau (SEQ ID NO: 3401), UGAguaggca (SEQ ID NO: 3402), UCAguaugua (SEQ ID NO: 3403), GAGguaucug (SEQ ID NO: 1954), AGAguauuuu (SEQ ID NO: 3404), AAGguuguag (SEQ ID NO: 3405), AGUguaaguu (SEQ ID NO: 821), CGGguaaguu (SEQ ID NO: 1626), UCGgugegga (SEQ ID NO: 3406), UAGguaagua (SEQ ID NO: 2491), GAAguuagau (SEQ ID NO: 3407), GCUgugagac (SEQ ID NO: 3408), CAGgcaggua (SEQ ID NO: 3409), CAGguagggg (SEQ ID NO: 1218), UAAguuaaga (SEQ ID NO: 3410), AUGguggguu (SEQ ID NO: 970), UAGguaaguu (SEQ ID NO: 2494), CUGguaaauu (SEQ ID NO: 1690), CCGguaagga (SEQ ID NO: 1577), GAGgcaggca (SEQ ID NO: 3411), CAUguaagug (SEQ ID NO: 1523), AAGgugccua (SEQ ID NO: 3412), UUGguaggga (SEQ ID NO: 2977), AAGguaaaca (SEQ ID NO: 123), CGGgugugag (SEQ ID NO: 3413), GGGgugugag (SEQ ID NO: 3414), UCCguggguc (SEQ ID NO: 3415), ACGguaaauc (SEQ ID NO: 3416), UCAguaggua (SEQ ID NO: 3417), CAGgucagcc (SEQ ID NO: 1278), CAGgeggugg (SEQ ID NO: 3418), CGAguaagcu (SEQ ID NO: 3419), CCCgugagca (SEQ ID NO: 3420), AAAguaauga (SEQ ID NO: 3421), CUGguaagcu (SEQ ID NO: 1702), CGGguaacca (SEQ ID NO: 3422), CAGgucgcac (SEQ ID NO: 3423), GAGguaggcc (SEQ ID NO: 3424), UAGgugagcc (SEQ ID NO: 2591), UAGguaggca (SEQ ID NO: 3425), GCGgugcgug (SEQ ID NO: 3426), AUGgugagua (SEQ ID NO: 961), GGGgugaggg (SEQ ID NO: 2282), GAGgucacac (SEQ ID NO: 3427), CAGguaggcc (SEQ ID NO: 3428), CAAgugcuga (SEQ ID NO: 3429), GUCgucuuca (SEQ ID NO: 3430), CAUguaagaa (SEQ ID NO: 1518), GUAguaagga (SEQ ID NO: 3431), UAGguuugua (SEQ ID NO: 2643), CAAguuagag (SEQ ID NO: 3432), AAGguagagu (SEQ ID NO: 208), AAGgugagau (SEQ ID NO: 338), AAAguaggua (SEQ ID NO: 37), ACAgugaauc (SEQ ID NO: 3433), CAGgugugeg (SEQ ID NO: 1436), CAGgucggcc (SEQ ID NO: 1299), AAGguaguau (SEQ ID NO: 3434), ACUgucaguc (SEQ ID NO: 3435), UCUgcagccu (SEQ ID NO: 3436), CGAguaagug (SEQ ID NO: 3437), AGAguaauua (SEQ ID NO: 3438), AGUgugagug (SEQ ID NO: 837), CCGgugageg (SEQ ID NO: 3439), AAGguaaccu (SEQ ID NO: 3440), AAGguugugg (SEQ ID NO: 3441), AAGgcauggg (SEQ ID NO: 3442), AAGgucagag (SEQ ID NO: 284), ACGguaaggu (SEQ ID NO: 3443), GGGgugagca (SEQ ID NO: 3444), GAGguugcuu (SEQ ID NO: 3445), AAGguaucgc (SEQ ID NO: 3446), CCGguaaagg (SEQ ID NO: 3447), AAAguuaaug (SEQ ID NO: 3448), UAGguacgag (SEQ ID NO: 2510), ACCguaauua (SEQ ID NO: 3449), GGGguaagga (SEQ ID NO: 2249), CCGguaacgc (SEQ ID NO: 3450), CAGgucagaa (SEQ ID NO: 1275), AAGguacuga (SEQ ID NO: 197), GAGgugacca (SEQ ID NO: 2010), GGGgugagcc (SEQ ID NO: 2277), AAGguacagg (SEQ ID NO: 177), AUGguaauua (SEQ ID NO: 3451), CAGgugagag (SEQ ID NO: 1335), AAGgugacuc (SEQ ID NO: 3452), AUAguaagua (SEQ ID NO: 849), GAGguaaacc (SEQ ID NO: 1869), CAGgugggau (SEQ ID NO: 1405), CAGgugagaa (SEQ ID NO: 1333), AGGguaaaaa (SEQ ID NO: 3453), GAGgugugac (SEQ ID NO: 3454), CACguaagcu (SEQ ID NO: 3455), CAGguccccc (SEQ ID NO: 3456), CAGgucaggu (SEQ ID NO: 3457), CGGguaaguc (SEQ ID NO: 3458), ACGguauggg (SEQ ID NO: 3459), GAUguaaguu (SEQ ID NO: 2123), CAAguaauau (SEQ ID NO: 3460), CAGguugggg (SEQ ID NO: 3461), CCUgugcugg (SEQ ID NO: 3462), AAGguaugau (SEQ ID NO: 256), AGGguagagg (SEQ ID NO: 3463), AAGguggguu (SEQ ID NO: 386), CAGgugugaa (SEQ ID NO: 1430), UUGguaugug (SEQ ID NO: 2988), UUGguaucuc (SEQ ID NO: 2985), GGGgugagug (SEQ ID NO: 2284), CUGgugugug (SEQ ID NO: 1779), AGGguagggc (SEQ ID NO: 3464), GUGgugagua (SEQ ID NO: 3465), CAGguaugua (SEQ ID NO: 1258), AAGguacauu (SEQ ID NO: 181), UUAguaagug (SEQ ID NO: 2934), AAUguauauc (SEQ ID NO: 3466), CUUguaagua (SEQ ID NO: 1793), GAGguuagua (SEQ ID NO: 2087), CAGguaaggu (SEQ ID NO: 1146), CAGguaaugu (SEQ ID NO: 1155), AGGgugaggc (SEQ ID NO: 3467), CAGguauuuc (SEQ ID NO: 1269), CAGgucugga (SEQ ID NO: 1307), GGGgugugcu (SEQ ID NO: 3468), UAGgugagug (SEQ ID NO: 2598), AAUguaaccu (SEQ ID NO: 3469), UAAgugaguc (SEQ ID NO: 2439), CAGgugcacu (SEQ ID NO: 3470), ACGguaagua (SEQ ID NO: 579), GAGguauccu (SEQ ID NO: 3471), UCUguaaguc (SEQ ID NO: 2745), CAGguauuca (SEQ ID NO: 1263), UGUguaagug (SEQ ID NO: 3472), CCAgcaaggc (SEQ ID NO: 3473), GAGgugaagg (SEQ ID NO: 2006), AAUguggggu (SEQ ID NO: 3474), UCGgugcgug (SEQ ID NO: 3475), UUGguaaggc (SEQ ID NO: 3476), GAGguaagug (SEQ ID NO: 3477), AAAguaagau (SEQ ID NO: 14), UAGgucuuuu (SEQ ID NO: 3478), GAGgucugau (SEQ ID NO: 3479), CCAguuagag (SEQ ID NO: 3480), UGGgugaaaa (SEQ ID NO: 3481), AGAguaagau (SEQ ID NO: 662), CAGguaauug (SEQ ID NO: 1158), CAGgcegguc (SEQ ID NO: 3482), CCGguaagag (SEQ ID NO: 3483), GAGgugagcu (SEQ ID NO: 2021), CUGguaagac (SEQ ID NO: 3484), CAGgugagau (SEQ ID NO: 1336), CUGguuuguu (SEQ ID NO: 3485), UGGguaggua (SEQ ID NO: 3486), CAGguuagug (SEQ ID NO: 1457), CAGguguucg (SEQ ID NO: 3487), CGGguagguc (SEQ ID NO: 3488), GUGguacaua (SEQ ID NO: 3489), AAGguacuaa (SEQ ID NO: 194), GAUgugagua (SEQ ID NO: 3490), UGUguaagac (SEQ ID NO: 2904), GAGguagccg (SEQ ID NO: 3491), UAGgugaucu (SEQ ID NO: 3492), CAGguacgug (SEQ ID NO: 1185), CUUgucaguc (SEQ ID NO: 3493), GAGguaucac (SEQ ID NO: 3494), GAGguaauga (SEQ ID NO: 3495), AAGguaacac (SEQ ID NO: 3496), CAGguaaagc (SEQ ID NO: 1123), AAGgcaagua (SEQ ID NO: 3497), CGCgugagcc (SEQ ID NO: 3498), AGUgugeguu (SEQ ID NO: 3499), GAUguaagca (SEQ ID NO: 2118), AAGguaauag (SEQ ID NO: 159), GGAgcaguug (SEQ ID NO: 3500), AGCguaagau (SEQ ID NO: 3501), AAGgucaggc (SEQ ID NO: 290), GAGguauuca (SEQ ID NO: 3502), AAUguaaagu (SEQ ID NO: 3503), CAGguaacaa (SEQ ID NO: 3504), UCGguaggug (SEQ ID NO: 3505), AAAguaaguc (SEQ ID NO: 22), CGGgugcagu (SEQ ID NO: 3506), GGUgugugca (SEQ ID NO: 3507), UGAgugagaa (SEQ ID NO: 2794), CACguguaag (SEQ ID NO: 3508), GUGguuggua (SEQ ID NO: 3509), GCAgccuuga (SEQ ID NO: 3510), CGAgugugau (SEQ ID NO: 3511), CAGguauaua (SEQ ID NO: 3512), UAUguaugug (SEQ ID NO: 2665), CCCgugguca (SEQ ID NO: 3513), AUGguaagac (SEQ ID NO: 890), GAGgugugga (SEQ ID NO: 2074), AGUguauccu (SEQ ID NO: 3514), UGAguguguc (SEQ ID NO: 3515), UGGguaaucu (SEQ ID NO: 3516), AUGgcagguu (SEQ ID NO: 3517), GAGguaagau (SEQ ID NO: 1884), UCAgcagcgu (SEQ ID NO: 3518), AAGgugggau (SEQ ID NO: 378), CGGgugcgcu (SEQ ID NO: 3519), CAGgugucug (SEQ ID NO: 1429), AGCgugguaa (SEQ ID NO: 3520), AAUgugaaug (SEQ ID NO: 3521), UCGgugagac (SEQ ID NO: 3522), UAGguaaage (SEQ ID NO: 3523), CUGguaaaag (SEQ ID NO: 3524), CCGgugegga (SEQ ID NO: 3525), CAGguacuca (SEQ ID NO: 3526), CAGguagcaa (SEQ ID NO: 1203), GAAguugagu (SEQ ID NO: 3527), GAGguggagg (SEQ ID NO: 2052), AGGguaugag (SEQ ID NO: 762), UAGguaugcu (SEQ ID NO: 3528), UAGgugagac (SEQ ID NO: 2588), CAGguaauua (SEQ ID NO: 1156), CGUguaagcc (SEQ ID NO: 3529), CUUguaaguu (SEQ ID NO: 1795), AAGguaacuu (SEQ ID NO: 140), UCGgcaaggc (SEQ ID NO: 3530), GAGguucucg (SEQ ID NO: 3531), GAGgugggcg (SEQ ID NO: 2058), AAGgcaugug (SEQ ID NO: 3532), CUGguauguu (SEQ ID NO: 1738), UAAgucauuu (SEQ ID NO: 3533), CAUguaauua (SEQ ID NO: 1525), AAUguaaaga (SEQ ID NO: 3534), UAGgugcuca (SEQ ID NO: 3535), AAGguaaugg (SEQ ID NO: 166), GAGguacuga (SEQ ID NO: 3536), UGGguaagua (SEQ ID NO: 2841), UGGguaaaaa (SEQ ID NO: 3537), AAGgugagcu (SEQ ID NO: 342), UACgugaguu (SEQ ID NO: 3538), AGGgugagcc (SEQ ID NO: 790), CGGgugagga (SEQ ID NO: 3539), UGGgugagag (SEQ ID NO: 2869), GGUguaagcu (SEQ ID NO: 3540), CGGguggguu (SEQ ID NO: 1648), CCAgcuaagu (SEQ ID NO: 3541), AAGguuuguc (SEQ ID NO: 467), GAGguuagac (SEQ ID NO: 2084), GAGguaccuc (SEQ ID NO: 3542), UUUguaaguu (SEQ ID NO: 3041), GAGguuagga (SEQ ID NO: 3543), CAGguaggga (SEQ ID NO: 1216), AGGguaauac (SEQ ID NO: 744), UGCgugugua (SEQ ID NO: 3544), CCAguaacca (SEQ ID NO: 3545), AGGgucuguc (SEQ ID NO: 3546), UGGguaugua (SEQ ID NO: 2860), GUGguaagcu (SEQ ID NO: 2348), CAGguaaccu (SEQ ID NO: 3547), AAGgugaguu (SEQ ID NO: 350), UAGguucgug (SEQ ID NO: 3548), AAAguuagua (SEQ ID NO: 3549), UGGgcaaguc (SEQ ID NO: 2816), AAGgcacagu (SEQ ID NO: 3550), GUUguaaguc (SEQ ID NO: 2401), AAGguuugcc (SEQ ID NO: 462), CUUgcauggg (SEQ ID NO: 3551), GCGgugagua (SEQ ID NO: 3552), GGGguaagcg (SEQ ID NO: 3553), GCCguaagaa (SEQ ID NO: 3554), GAGgucggga (SEQ ID NO: 3555), UUGguauugu (SEQ ID NO: 2990), AGUgugagac (SEQ ID NO: 3556), CUGgugggga (SEQ ID NO: 1770), AGAguaaggu (SEQ ID NO: 668), CCGguggguc (SEQ ID NO: 3557), CAGguauucu (SEQ ID NO: 1264), UGGguaacgu (SEQ ID NO: 3558), UUGgugagag (SEQ ID NO: 3559), UAGguacccu (SEQ ID NO: 3560), GGGgugcguc (SEQ ID NO: 3561), AAGgcaggag (SEQ ID NO: 3562), ACGguacauu (SEQ ID NO: 3563), GAGguaguua (SEQ ID NO: 1946), CAGguauggg (SEQ ID NO: 1256), UUUguguguc (SEQ ID NO: 3053), CAGguacuua (SEQ ID NO: 1194), AUGguauacu (SEQ ID NO: 3564), AGUgugagcc (SEQ ID NO: 833), ACAguaacga (SEQ ID NO: 3565), CUGguaccca (SEQ ID NO: 3566), CAGguaaccc (SEQ ID NO: 3567), GGAguaagua (SEQ ID NO: 3568), GAGgugggug (SEQ ID NO: 2065), ACUguauguc (SEQ ID NO: 3569), ACGgugagua (SEQ ID NO: 606), CUGguaaugu (SEQ ID NO: 3570), AAGguaucag (SEQ ID NO: 247), CAGgugcccc (SEQ ID NO: 1370), AGUgucagug (SEQ ID NO: 3571), AAGguaggag (SEQ ID NO: 218), GGAguaugug (SEQ ID NO: 3572), UUGguauuuu (SEQ ID NO: 2992), CCUguuguga (SEQ ID NO: 3573), UUUguaagaa (SEQ ID NO: 3033), UAGguaacau (SEQ ID NO: 2475), CAGguaagca (SEQ ID NO: 3574), CAGgucacag (SEQ ID NO: 3575), CAGgugugag (SEQ ID NO: 1432), UAGguuugcg (SEQ ID NO: 3576), CUGguaagaa (SEQ ID NO: 1697), ACGguuguau (SEQ ID NO: 3577), AAGguugggg (SEQ ID NO: 446), AAGgugaauu (SEQ ID NO: 329), GGGguuaguu (SEQ ID NO: 3578), ACGguaaggc (SEQ ID NO: 3579), CAGguuuaag (SEQ ID NO: 1496), CUGguaaguu (SEQ ID NO: 1709), GGGgugagag (SEQ ID NO: 3580), UGGguggguu (SEQ ID NO: 2886), GAGguuuguu (SEQ ID NO: 2111), UGGguaaaug (SEQ ID NO: 2826), CAGgcaggcc (SEQ ID NO: 3581), CACgugcagg (SEQ ID NO: 3582), AAGgugagcc (SEQ ID NO: 340), CAAguaagug (SEQ ID NO: 1028), CAGgucaguc (SEQ ID NO: 1282), GCGguauaau (SEQ ID NO: 3583), UAGguaaagu (SEQ ID NO: 3584), UAGguggauu (SEQ ID NO: 3585), GAGgucugga (SEQ ID NO: 3586), UCGgucaguu (SEQ ID NO: 3587), UGGguaacug (SEQ ID NO: 3588), AAGguuugau (SEQ ID NO: 3589), UGUgcuggug (SEQ ID NO: 3590), UGUguaccuc (SEQ ID NO: 3591), UGGguacagu (SEQ ID NO: 2849), AUCgucageg (SEQ ID NO: 3592), CAGgucuugg (SEQ ID NO: 3593), GAAguuggua (SEQ ID NO: 3594), GAAguaaaga (SEQ ID NO: 3595), UUGguaagcu (SEQ ID NO: 2959), UAGguaccag (SEQ ID NO: 2507), AGGguaucau (SEQ ID NO: 3596), CAGguaaaaa (SEQ ID NO: 1118), ACGguaauuu (SEQ ID NO: 583), AUUguaaguu (SEQ ID NO: 997), GAGguacagu (SEQ ID NO: 1908), CAGgugaaag (SEQ ID NO: 1315), UGGguuguuu (SEQ ID NO: 3597), GGGguaggug (SEQ ID NO: 2259), CAGgugccca (SEQ ID NO: 1369), AGCgugagau (SEQ ID NO: 3598), CCAgugagug (SEQ ID NO: 1565), AGGguagaug (SEQ ID NO: 3599), UGGguguguc (SEQ ID NO: 2888), AUCgcgugag (SEQ ID NO: 3600), AGGguaagcc (SEQ ID NO: 3601), AGGguagcag (SEQ ID NO: 3602), UUCguuuccg (SEQ ID NO: 3603), AAGguaagcg (SEQ ID NO: 147), UGGguaagcc (SEQ ID NO: 2837), CAGguauggc (SEQ ID NO: 3604), UGUguaagua (SEQ ID NO: 2907), AAGguagaga (SEQ ID NO: 3605), ACGguaauaa (SEQ ID NO: 3606), CUGguacggu (SEQ ID NO: 3607), GAGgucacag (SEQ ID NO: 3608), UAUguaaguu (SEQ ID NO: 2656), CUGguacgcc (SEQ ID NO: 3609), CAAguaagau (SEQ ID NO: 1024), CUAgugagua (SEQ ID NO: 1673), CCGguaaccg (SEQ ID NO: 3610), CUUguaaguc (SEQ ID NO: 3611), GUGgugagaa (SEQ ID NO: 2378), ACCguaugua (SEQ ID NO: 3612), GUAguaagug (SEQ ID NO: 2324), UUGgugggua (SEQ ID NO: 3014), CGGguacuuu (SEQ ID NO: 3613), UGGguaaaua (SEQ ID NO: 2825), AGAgugagua (SEQ ID NO: 704), AAGguagguu (SEQ ID NO: 230), AAGguaugcg (SEQ ID NO: 3614), CCUguaggcu (SEQ ID NO: 3615), ACAguagaaa (SEQ ID NO: 3616), CCGguuagua (SEQ ID NO: 3617), CGGguaggcg (SEQ ID NO: 3618), GCAgugagug (SEQ ID NO: 2162), GAGgugaguc (SEQ ID NO: 3619), CUGguagccu (SEQ ID NO: 3620), CAUguaugua (SEQ ID NO: 1533), GAAguaacuu (SEQ ID NO: 3621), GAAguaagau (SEQ ID NO: 3622), AAGguuagau (SEQ ID NO: 417), AAGguaauca (SEQ ID NO: 161), AAUguaugua (SEQ ID NO: 507), UGAguaagau (SEQ ID NO: 2767), AGAgugagca (SEQ ID NO: 703), GUAguucuau (SEQ ID NO: 3623), GAGguaauca (SEQ ID NO: 1898), UAGguaugga (SEQ ID NO: 2548), UAGgugggac (SEQ ID NO: 2612), GAGguacaug (SEQ ID NO: 3624), UGGguaaggc (SEQ ID NO: 3625), CAGguacgcc (SEQ ID NO: 1182), CCAguuacgc (SEQ ID NO: 3626), ACUgugguga (SEQ ID NO: 3627), GAGguaaguc (SEQ ID NO: 1894), AUUguaggug (SEQ ID NO: 3628), ACCgucagug (SEQ ID NO: 3629), AAUgugaggg (SEQ ID NO: 3630), ACUgugagug (SEQ ID NO: 645), UGGguguggu (SEQ ID NO: 3631), AAGguuggga (SEQ ID NO: 445), AAGguuugga (SEQ ID NO: 464), UCCgugagug (SEQ ID NO: 3632), CGGgugagug (SEQ ID NO: 1642), AGAguaagcu (SEQ ID NO: 664), CAGgcaagcu (SEQ ID NO: 3633), UAGguauauu (SEQ ID NO: 2541), AAAguagcag (SEQ ID NO: 3634), GAGguaaccu (SEQ ID NO: 1880), AAGgugggca (SEQ ID NO: 379), AGGgugagua (SEQ ID NO: 795), UGGguaaggu (SEQ ID NO: 2840), CUUgucagug (SEQ ID NO: 3635), UAGgugcgcu (SEQ ID NO: 3636), GAGgcaaauu (SEQ ID NO: 3637), AGGguaccuc (SEQ ID NO: 3638), CAAgugcgua (SEQ ID NO: 3639), AGAguaagac (SEQ ID NO: 660), GUGguaaaua (SEQ ID NO: 3640), GAUguaagcg (SEQ ID NO: 3641), GAGguaaagc (SEQ ID NO: 1871), UAGgugagua (SEQ ID NO: 2596), CAGguaacau (SEQ ID NO: 1130), CCUguacggc (SEQ ID NO: 3642), UAGguauguc (SEQ ID NO: 2552), UAGguccaua (SEQ ID NO: 3643), GAGgugaaaa (SEQ ID NO: 2003), AAAguacuga (SEQ ID NO: 3644), UUGguaagcg (SEQ ID NO: 3645), CAGgcaageg (SEQ ID NO: 3646), UUUgcagguu (SEQ ID NO: 3647), CAGguuuaua (SEQ ID NO: 3648), CUGguaaagc (SEQ ID NO: 1686), AUGgugagcu (SEQ ID NO: 958), CAGgugguug (SEQ ID NO: 1419), GUAguaaguu (SEQ ID NO: 3649), CAGguaauac (SEQ ID NO: 3650), CAGgcaaggc (SEQ ID NO: 3651), AAGguaauuu (SEQ ID NO: 171), UUUguccgug (SEQ ID NO: 3652), GAGguagguu (SEQ ID NO: 1939), ACCgugagug (SEQ ID NO: 3653), CAAguaagcu (SEQ ID NO: 3654), ACAgugagua (SEQ ID NO: 560), UUGgugagau (SEQ ID NO: 3000), AAGguagucu (SEQ ID NO: 233), CAGguaaagg (SEQ ID NO: 3655), GGGguaugga (SEQ ID NO: 2264), UUUguaagug (SEQ ID NO: 3040), GUGguaagag (SEQ ID NO: 2344), AGUgugaguu (SEQ ID NO: 838), AAGgcaageg (SEQ ID NO: 3656), UAAgugagua (SEQ ID NO: 2438), AGGgugagug (SEQ ID NO: 797), AGUguacgug (SEQ ID NO: 3657), AGGgugcgua (SEQ ID NO: 3658), GGCgugagcc (SEQ ID NO: 2238), CGAguuauga (SEQ ID NO: 3659), CAGguaaaga (SEQ ID NO: 1122), UUGgugaaga (SEQ ID NO: 3660), AGGguaaugg (SEQ ID NO: 3661), AAGguccaga (SEQ ID NO: 300), AGUgugaguc (SEQ ID NO: 836), CAGguaauuu (SEQ ID NO: 1159), CAGguaacgc (SEQ ID NO: 3662), CUGguacacu (SEQ ID NO: 3663), CUGguuagug (SEQ ID NO: 1782), CAGguacuug (SEQ ID NO: 3664), CACguaagua (SEQ ID NO: 3665), GUGgugcggc (SEQ ID NO: 3666), GAGgucaguu (SEQ ID NO: 3667), AUGguaugcc (SEQ ID NO: 932), AAGgugugug (SEQ ID NO: 405), CUGguggguc (SEQ ID NO: 1772), CAGgugaggc (SEQ ID NO: 1342), AAGguuaguc (SEQ ID NO: 423), AAGguagcug (SEQ ID NO: 215), GAGgucagga (SEQ ID NO: 1983), GUUguaggua (SEQ ID NO: 3668), UGGguacaag (SEQ ID NO: 3669), AUGguaggug (SEQ ID NO: 924), GAGguaagcc (SEQ ID NO: 1886), AUGgcaagua (SEQ ID NO: 3670), AAGguauauu (SEQ ID NO: 245), GCGgugagag (SEQ ID NO: 3671), AAGgugcuuc (SEQ ID NO: 3672), UAGguacauc (SEQ ID NO: 3673), ACUgugguaa (SEQ ID NO: 3674), GAGguaggcu (SEQ ID NO: 1933), GAGguaugca (SEQ ID NO: 3675), AGGguaguuc (SEQ ID NO: 3676), CAGguauccu (SEQ ID NO: 1241), AGGguaaguc (SEQ ID NO: 741), AGGgucaguu (SEQ ID NO: 779), CAGguuggga (SEQ ID NO: 3677), CAGguggaua (SEQ ID NO: 3678), GGAguagguu (SEQ ID NO: 2220), GAGguaggau (SEQ ID NO: 3679), GGGguuugug (SEQ ID NO: 3680), UAGguaauug (SEQ ID NO: 3681), AAGguaaccc (SEQ ID NO: 136), ACGguaagaa (SEQ ID NO: 3682), GAGguagggg (SEQ ID NO: 1936), CGAguaggug (SEQ ID NO: 1619), UCCguaagug (SEQ ID NO: 2710), UCGguacagg (SEQ ID NO: 3683), CAAguaageg (SEQ ID NO: 3684), AAGguccgeg (SEQ ID NO: 3685), AAUgugagua (SEQ ID NO: 523), CAGgugaaug (SEQ ID NO: 3686), GUGguaaggc (SEQ ID NO: 2350), AGAgugagug (SEQ ID NO: 706), UCUguauguc (SEQ ID NO: 3687), UGGgugaguc (SEQ ID NO: 2876), UCGguuagua (SEQ ID NO: 3688), GAUguaugca (SEQ ID NO: 3689), GAGguuggug (SEQ ID NO: 3690), GAGguggggc (SEQ ID NO: 2061), UGGgucaguc (SEQ ID NO: 3691), GCAgugagua (SEQ ID NO: 2161), CAGguugcuu (SEQ ID NO: 3692), AGGguagagu (SEQ ID NO: 3693), UAGgucaggu (SEQ ID NO: 2567), CGCguaugua (SEQ ID NO: 3694), GAGguauuaa (SEQ ID NO: 3695), CAGguaaacu (SEQ ID NO: 3696), AAAguaaguu (SEQ ID NO: 24), GGGgucuggc (SEQ ID NO: 3697), GCUguggggu (SEQ ID NO: 3698), UUGguaaguc (SEQ ID NO: 3699), AAGguagaag (SEQ ID NO: 3700), AAUgugaguc (SEQ ID NO: 524), AAGgucagcu (SEQ ID NO: 288), AAGguaagag (SEQ ID NO: 143), AUGgugagga (SEQ ID NO: 3701), AAGguacuuc (SEQ ID NO: 200), AAGguaagaa (SEQ ID NO: 141), CCGguacagc (SEQ ID NO: 3702), GCGgugcgga (SEQ ID NO: 3703), CAGguacaua (SEQ ID NO: 1168), CUGgugagga (SEQ ID NO: 1755), CUGguaggug (SEQ ID NO: 1731), AACguagguu (SEQ ID NO: 3704), AUGgugugug (SEQ ID NO: 975), UUGguacuau (SEQ ID NO: 3705), CAGgucggug (SEQ ID NO: 1300), CAGgcauggg (SEQ ID NO: 3706), AUGguaucuu (SEQ ID NO: 929), AAGguaacua (SEQ ID NO: 137), CAGgugggeg (SEQ ID NO: 3707), CACgugagga (SEQ ID NO: 3708), AAGgugguuc (SEQ ID NO: 392), UGGgcauucu (SEQ ID NO: 3709), AUGguaagcc (SEQ ID NO: 894), AGGgucagug (SEQ ID NO: 778), AGAguacgua (SEQ ID NO: 3710), AAGguaggca (SEQ ID NO: 220), AAGguauuca (SEQ ID NO: 3711), CAGguagauu (SEQ ID NO: 1202), GAGguauuua (SEQ ID NO: 1972), GAGgucuaca (SEQ ID NO: 3712), GUUguagguc (SEQ ID NO: 3713), CAGguacucg (SEQ ID NO: 3714), GUCguauguu (SEQ ID NO: 3715), AAGguacuuu (SEQ ID NO: 202), AGAgugagau (SEQ ID NO: 702), AGUguuggua (SEQ ID NO: 3716), AAUgugagug (SEQ ID NO: 525), AAGguagauu (SEQ ID NO: 3717), AUGguuugua (SEQ ID NO: 988), GAGgccccag (SEQ ID NO: 3718), AUGgucaguu (SEQ ID NO: 3719), UCUguaagga (SEQ ID NO: 3720), CAGgucgggc (SEQ ID NO: 3721), CAGguaagcc (SEQ ID NO: 1142), UAGgucagug (SEQ ID NO: 2569), AGAguaggaa (SEQ ID NO: 683), CUGguacuuc (SEQ ID NO: 3722), CUCguaagca (SEQ ID NO: 1674), CAGguaacua (SEQ ID NO: 1134), CAGguggcug (SEQ ID NO: 1401), UGGguccgua (SEQ ID NO: 3723), GAGguugugc (SEQ ID NO: 3724), CAGgugegcg (SEQ ID NO: 1377), AAAguauggc (SEQ ID NO: 3725), UGAguacgua (SEQ ID NO: 2779), CUGguacgga (SEQ ID NO: 3726), CAAgugaccu (SEQ ID NO: 3727), AAGgugaugu (SEQ ID NO: 356), AAGgucugca (SEQ ID NO: 3728), AAAguuugua (SEQ ID NO: 75), AAGgugagca (SEQ ID NO: 339), GAUguaagcc (SEQ ID NO: 2119), CAAguaauuu (SEQ ID NO: 1035), CAGgugugug (SEQ ID NO: 1442), UGGgugaggg (SEQ ID NO: 2874), AAGgugaccu (SEQ ID NO: 3729), UAGgugugag (SEQ ID NO: 2621), CAGgcagguc (SEQ ID NO: 3730), UCAguaaguu (SEQ ID NO: 2692), UCAgcaguga (SEQ ID NO: 3731), AAGguaccac (SEQ ID NO: 3732), UAAguaggug (SEQ ID NO: 3733), AAGgucagcc (SEQ ID NO: 286), CAGguaacuc (SEQ ID NO: 1135), AAAguaagag (SEQ ID NO: 13), AAGguagaua (SEQ ID NO: 209), AAGgcaaggg (SEQ ID NO: 99), CAGgugucgg (SEQ ID NO: 3734), CAGguggcua (SEQ ID NO: 3735), GAGguugcca (SEQ ID NO: 3736), CAGgccgugg (SEQ ID NO: 3737), UUGguauaug (SEQ ID NO: 3738), GAGguugagu (SEQ ID NO: 3739), GAGguagguc (SEQ ID NO: 3740), GUGguaagac (SEQ ID NO: 2343), UAGguccuuc (SEQ ID NO: 3741), GAGgcaaguc (SEQ ID NO: 3742), GAGguaacau (SEQ ID NO: 3743), CAGguauauc (SEQ ID NO: 1236), UCGguugguu (SEQ ID NO: 3744), CAGgugaacc (SEQ ID NO: 3745), CAGgucuuuu (SEQ ID NO: 3746), CAGgcauggc (SEQ ID NO: 3747), AAAguacuug (SEQ ID NO: 32), CAGgugauuc (SEQ ID NO: 1356), UUGguagguu (SEQ ID NO: 3748), UAUgugagca (SEQ ID NO: 3749), CAGgugageg (SEQ ID NO: 1339), AAUguaauaa (SEQ ID NO: 3750), AAAguaaggc (SEQ ID NO: 3751), UAGguuuguc (SEQ ID NO: 2644), UAGgugggag (SEQ ID NO: 2613), GAGguaaguu (SEQ ID NO: 3752), AAGguagccg (SEQ ID NO: 3753), CAGguggugc (SEQ ID NO: 3754), UGAgucaguu (SEQ ID NO: 3755), CUGguaggcc (SEQ ID NO: 3756), CAAguaagga (SEQ ID NO: 3757), CGGguaaggc (SEQ ID NO: 3758), AAGgcgagga (SEQ ID NO: 3759), CAGguaguuc (SEQ ID NO: 1230), CAGguaagga (SEQ ID NO: 1143), CCUgugagug (SEQ ID NO: 1610), AAGguaaaug (SEQ ID NO: 132), CCGguaauua (SEQ ID NO: 3760), CAGguaaguu (SEQ ID NO: 1149), AAGgugguca (SEQ ID NO: 3761), CAGguaccuc (SEQ ID NO: 1177), AUCguaagua (SEQ ID NO: 3762), CCGguacaua (SEQ ID NO: 3763), GCGgugagug (SEQ ID NO: 3764), GAGgugguau (SEQ ID NO: 2067), CUGgugugga (SEQ ID NO: 3765), GAGguaauuc (SEQ ID NO: 3766), CAAguacgua (SEQ ID NO: 3767), UCUguaagug (SEQ ID NO: 2746), AAUguaagug (SEQ ID NO: 491), AGGgucuguu (SEQ ID NO: 783), GAGguacugc (SEQ ID NO: 1918), AGGguaaggc (SEQ ID NO: 738), AAGgcaagag (SEQ ID NO: 95), CAGguggguu (SEQ ID NO: 1416), UAGguuagga (SEQ ID NO: 3768), UGAguaagcu (SEQ ID NO: 2769), AGAguaagag (SEQ ID NO: 661), AUGgcaggug (SEQ ID NO: 3769), UAGgcaagua (SEQ ID NO: 3770), AUGguaggua (SEQ ID NO: 923), GCAgcccgca (SEQ ID NO: 3771), ACGguaaacu (SEQ ID NO: 3772), AGGgugaguu (SEQ ID NO: 798), GUAguagucu (SEQ ID NO: 3773), GUGgcugaaa (SEQ ID NO: 3774), CAGguuaguc (SEQ ID NO: 1456), CUGgugagca (SEQ ID NO: 1753), UCAguaagug (SEQ ID NO: 2691), AAAgugauug (SEQ ID NO: 3775), UAGgucugga (SEQ ID NO: 3776), GAGguguuuc (SEQ ID NO: 3777), AAGguaaauu (SEQ ID NO: 133), CAUguacauc (SEQ ID NO: 3778), AAGguuugaa (SEQ ID NO: 3779), CCAgcaagug (SEQ ID NO: 3780), UAGguaauaa (SEQ ID NO: 3781), GAGgcaagug (SEQ ID NO: 1859), CAAgugauuc (SEQ ID NO: 1071), CAGgucgugg (SEQ ID NO: 3782), GAAguaugcc (SEQ ID NO: 3783), UCGgugcccu (SEQ ID NO: 3784), GAGgucaguc (SEQ ID NO: 3785), CAGgugagac (SEQ ID NO: 1334), UUUgucugua (SEQ ID NO: 3786), CAGguagaua (SEQ ID NO: 3787), UGGguaucag (SEQ ID NO: 3788), UAGgugggcu (SEQ ID NO: 2616), AUGgugagau (SEQ ID NO: 3789), CAGguaacac (SEQ ID NO: 3790), CCGguauccu (SEQ ID NO: 3791), UAGguaagcu (SEQ ID NO: 2487), UCAguacauc (SEQ ID NO: 3792), UAGguuugcc (SEQ ID NO: 2642), AUGguaagaa (SEQ ID NO: 889), UUGguaagac (SEQ ID NO: 3793), CCGguuaguc (SEQ ID NO: 3794), GAGguaagaa (SEQ ID NO: 1882), UGGguaaguu (SEQ ID NO: 2844), CCGgugagaa (SEQ ID NO: 1585), CCUgugaggg (SEQ ID NO: 1608), ACGguaggag (SEQ ID NO: 590), ACAguauguc (SEQ ID NO: 3795), CAGguauuaa (SEQ ID NO: 3796), CAGguggauc (SEQ ID NO: 3797), AGAgugcgua (SEQ ID NO: 3798), AAGgugaccg (SEQ ID NO: 3799), AGAguaggug (SEQ ID NO: 687), ACUguaugua (SEQ ID NO: 3800), UAGgucaauu (SEQ ID NO: 3801), AGUguguaag (SEQ ID NO: 3802), CGGguaccuu (SEQ ID NO: 3803), CUAgugaguu (SEQ ID NO: 3804), CUAguaagug (SEQ ID NO: 1666), CAGguacaac (SEQ ID NO: 3805), UAGgugugug (SEQ ID NO: 2627), CAUguacggc (SEQ ID NO: 3806), AUGgugugag (SEQ ID NO: 3807), AGGguggaag (SEQ ID NO: 3808), CAGgugcgag (SEQ ID NO: 3809), UAGgugcucc (SEQ ID NO: 3810), AAGguggugg (SEQ ID NO: 390), AAGgucuguu (SEQ ID NO: 317), CAGgugggcc (SEQ ID NO: 1407), AAGgucaguc (SEQ ID NO: 294), CAGguuuuua (SEQ ID NO: 3811), AACgugaggu (SEQ ID NO: 3812), CGGguaagag (SEQ ID NO: 3813), UUUgucggua (SEQ ID NO: 3814), UAGguuaagu (SEQ ID NO: 3815), GUGguaagaa (SEQ ID NO: 2342), CAGguauugg (SEQ ID NO: 1266), GCUguaaguu (SEQ ID NO: 2196), CUAguaagua (SEQ ID NO: 1664), UCGguaaaua (SEQ ID NO: 3816), CAGguaacuu (SEQ ID NO: 1137), CCUgugagua (SEQ ID NO: 3817), CAGguuauau (SEQ ID NO: 3818), CUGgugaaca (SEQ ID NO: 3819), AAGguauaaa (SEQ ID NO: 238), GAGguaagca (SEQ ID NO: 1885), AAGgugaagc (SEQ ID NO: 324), CAGgugaguu (SEQ ID NO: 1348), UUUgugagua (SEQ ID NO: 3820), CUUguacgcc (SEQ ID NO: 3821), AGAguaagug (SEQ ID NO: 670), UGGguaggug (SEQ ID NO: 2853), UGAgcccugc (SEQ ID NO: 3822), UGUguaugua (SEQ ID NO: 3823), AAGguagagg (SEQ ID NO: 3824), GAGguggggg (SEQ ID NO: 2062), UAGguaauuc (SEQ ID NO: 2502), AAGgcauggu (SEQ ID NO: 3825), AGAguaagca (SEQ ID NO: 663), AAGguaggaa (SEQ ID NO: 217), CAAguaagua (SEQ ID NO: 1026), ACUguaauug (SEQ ID NO: 3826), CAGgucugug (SEQ ID NO: 1311), UCGguaccga (SEQ ID NO: 3827), CUGgugagag (SEQ ID NO: 3828), AAGguuugcu (SEQ ID NO: 463), AUGguaccac (SEQ ID NO: 3829), UAAguuaguu (SEQ ID NO: 3830), CAGguaggac (SEQ ID NO: 1213), AGAgugaggc (SEQ ID NO: 3831), CGAgucagua (SEQ ID NO: 3832), CAGgucugag (SEQ ID NO: 1304), GAGguggugg (SEQ ID NO: 3833), ACGguauugg (SEQ ID NO: 3834), GCUgcgagua (SEQ ID NO: 3835), CUGguaagug (SEQ ID NO: 1708), GUGgugagau (SEQ ID NO: 2379), GGGguuugau (SEQ ID NO: 3836), UCUgugagug (SEQ ID NO: 2762), CUUgucagua (SEQ ID NO: 1801), GAGguaaaac (SEQ ID NO: 1866), UCUguaagau (SEQ ID NO: 2741), CCAguaaguu (SEQ ID NO: 1558), CAGguaaagu (SEQ ID NO: 1124), GCGgugagca (SEQ ID NO: 2179), UAAguaagag (SEQ ID NO: 2416), CUGgcaggug (SEQ ID NO: 3837), GAGguaaggg (SEQ ID NO: 1891), UGAguaaguu (SEQ ID NO: 2775), GAGgugagac (SEQ ID NO: 2015), GCUgucuguu (SEQ ID NO: 3838), AAGguaacaa (SEQ ID NO: 134), GAGguaacgg (SEQ ID NO: 3839), CUGguauucu (SEQ ID NO: 3840), CAAguaacug (SEQ ID NO: 1021), AAGguggggu (SEQ ID NO: 383), UAGguauggc (SEQ ID NO: 2549), CAGguauuuu (SEQ ID NO: 1271), GUGguaaacu (SEQ ID NO: 3841), GAGgucugag (SEQ ID NO: 1998), CUGguaaggu (SEQ ID NO: 1706), CAAguaaguu (SEQ ID NO: 1029), AAGguagacc (SEQ ID NO: 206), GAGgcgagcg (SEQ ID NO: 3842), CUGguaaaua (SEQ ID NO: 1687), UGUguaageg (SEQ ID NO: 3843), CAGguuaggg (SEQ ID NO: 1453), GGGgugagga (SEQ ID NO: 2280), ACAguaugug (SEQ ID NO: 3844), CCGgugggga (SEQ ID NO: 3845), GAGgucagug (SEQ ID NO: 3846), AGGguaaggu (SEQ ID NO: 3847), ACAguaagua (SEQ ID NO: 546), GGUguaaggu (SEQ ID NO: 3848), GAGguaauaa (SEQ ID NO: 1895), CAGguauucc (SEQ ID NO: 3849), CUGguauaaa (SEQ ID NO: 3850), CCGgucugug (SEQ ID NO: 3851), CAGguaacug (SEQ ID NO: 1136), GCAguaagua (SEQ ID NO: 2147), AAGguagggg (SEQ ID NO: 225), CAAguccacc (SEQ ID NO: 3852), CAAguuggug (SEQ ID NO: 3853), CAGgugeggu (SEQ ID NO: 1379), CAGguaaaau (SEQ ID NO: 3854), ACGguaagga (SEQ ID NO: 3855), UGGguaauaa (SEQ ID NO: 3856), UAGguaagug (SEQ ID NO: 2493), CCGguagguu (SEQ ID NO: 3857), AGAguaugga (SEQ ID NO: 3858), CUCgugaguc (SEQ ID NO: 3859), AAAgccggug (SEQ ID NO: 3860), UUGguaauuu (SEQ ID NO: 2970), GAGguaaaag (SEQ ID NO: 1867), CCUgugugag (SEQ ID NO: 3861), AAAguaagga (SEQ ID NO: 18), UGAgugagug (SEQ ID NO: 2800), AAGguacaug (SEQ ID NO: 180), CCGguaaaug (SEQ ID NO: 3862), CAGgugaagc (SEQ ID NO: 3863), CAGguacccg (SEQ ID NO: 1173), GAGguaaggc (SEQ ID NO: 1890), UUUguauguu (SEQ ID NO: 3049), CAGgugcucc (SEQ ID NO: 1386), UCGguagguc (SEQ ID NO: 3864), CGGgugaggc (SEQ ID NO: 3865), AAGguaauua (SEQ ID NO: 168), ACUgugaguc (SEQ ID NO: 644), AAGgucagca (SEQ ID NO: 285), GUGgugagug (SEQ ID NO: 2384), CAUguccacc (SEQ ID NO: 3866), AAGgugaccc (SEQ ID NO: 3867), CGGguuagua (SEQ ID NO: 3868), GCGguaguaa (SEQ ID NO: 3869), GCUguaggua (SEQ ID NO: 3870), CCUguugagu (SEQ ID NO: 3871), UAGgucuggc (SEQ ID NO: 2577), GAUgugagcc (SEQ ID NO: 2131), CUUgugagua (SEQ ID NO: 1802), CUGguguguu (SEQ ID NO: 1780), GAGgcaugug (SEQ ID NO: 1863), CAGgcaagag (SEQ ID NO: 1101), UUGguaagaa (SEQ ID NO: 2957), GAGguguggg (SEQ ID NO: 2075), GAGguauuuu (SEQ ID NO: 1975), CAGguaguaa (SEQ ID NO: 1224), AGGguaagac (SEQ ID NO: 3872), UUUguaggca (SEQ ID NO: 3873), AGGgugagau (SEQ ID NO: 3874), GAGguuugua (SEQ ID NO: 2110), AAGgugagug (SEQ ID NO: 349), GAGgugggag (SEQ ID NO: 2055), AAGgugagaa (SEQ ID NO: 335), CUGguaagag (SEQ ID NO: 1698), AUAguaaaga (SEQ ID NO: 3875), GAUgugaguc (SEQ ID NO: 2134), AAGgugcagg (SEQ ID NO: 3876), CAGgucuguc (SEQ ID NO: 1310), GAGgugauuu (SEQ ID NO: 3877), CAGguuggcu (SEQ ID NO: 3878), CGGguauggg (SEQ ID NO: 3879), AUGguccauc (SEQ ID NO: 3880), CCGguuggug (SEQ ID NO: 3881), GGAguaaguc (SEQ ID NO: 3882), AAUguaagga (SEQ ID NO: 488), CAGguuuguu (SEQ ID NO: 1510), UAGgugugua (SEQ ID NO: 2626), UAUgucuuug (SEQ ID NO: 3883), ACGguacuuc (SEQ ID NO: 3884), AAGgcacgcg (SEQ ID NO: 3885), CUGguaaace (SEQ ID NO: 1684), CUUgugggua (SEQ ID NO: 3886), UGAguaaguc (SEQ ID NO: 2773), CUGgugggug (SEQ ID NO: 1773), GAGguggaga (SEQ ID NO: 3887), GUGguggcug (SEQ ID NO: 3888), GUGguaagug (SEQ ID NO: 2353), AACgugagua (SEQ ID NO: 3889), GAAgcuguaa (SEQ ID NO: 3890), CGGguaucuu (SEQ ID NO: 3891), CAGgugucag (SEQ ID NO: 1424), AAUguacgca (SEQ ID NO: 3892), CCGgugggua (SEQ ID NO: 3893), UGGgugaggu (SEQ ID NO: 3894), AAGguauguu (SEQ ID NO: 266), CAGguauguu (SEQ ID NO: 1261), CAGguuugcu (SEQ ID NO: 1505), UUGguaaguu (SEQ ID NO: 2964), CAGguaguug (SEQ ID NO: 1231), CCUgugaaua (SEQ ID NO: 3895), GCUgugugug (SEQ ID NO: 3896), CAAguaauuc (SEQ ID NO: 1033), AGGguaaugu (SEQ ID NO: 3897), GCUgugaguc (SEQ ID NO: 2205), ACCguaaguu (SEQ ID NO: 3898), CGUguaagua (SEQ ID NO: 3899), GGGguaaguc (SEQ ID NO: 3900), AAUguaugau (SEQ ID NO: 3901), AAUgugauua (SEQ ID NO: 3902), UCAguaagaa (SEQ ID NO: 2682), CAGguccguc (SEQ ID NO: 3903), GAAguauuga (SEQ ID NO: 3904), UUGguaagga (SEQ ID NO: 2960), CAGgucgguu (SEQ ID NO: 3905), UAGguuagug (SEQ ID NO: 2635), ACGguaaaac (SEQ ID NO: 577), AAGguagguc (SEQ ID NO: 228), UACgugagua (SEQ ID NO: 3906), UUGguaagca (SEQ ID NO: 3907), GCGgugaguc (SEQ ID NO: 3908), GAAguaaggg (SEQ ID NO: 3909), CGCgugaguu (SEQ ID NO: 3910), CAGguacccc (SEQ ID NO: 3911), UCUguaagac (SEQ ID NO: 3912), GAGgugggca (SEQ ID NO: 2057), AAUguaagac (SEQ ID NO: 3913), CAGgcaaggg (SEQ ID NO: 3914), CAAguaacua (SEQ ID NO: 1020), AAAguuuguc (SEQ ID NO: 3915), CAGguacugu (SEQ ID NO: 1193), AAGgucccuc (SEQ ID NO: 303), UCGguaaguc (SEQ ID NO: 3916), UGGgugagug (SEQ ID NO: 2877), CUUgugagau (SEQ ID NO: 3917), AGAgugagcu (SEQ ID NO: 3918), UAAgugggga (SEQ ID NO: 3919), UAGguaggga (SEQ ID NO: 2522), CAGguuagcc (SEQ ID NO: 1452), AGGguaauca (SEQ ID NO: 3920), AAGguucagc (SEQ ID NO: 3921), UGGgugggug (SEQ ID NO: 2885), CAGguuguga (SEQ ID NO: 1494), AAGguaagug (SEQ ID NO: 155), CAUgugegua (SEQ ID NO: 1543), CCGguauauu (SEQ ID NO: 3922), ACCguaugug (SEQ ID NO: 3923), CAGguauagu (SEQ ID NO: 3924), CAGguauuac (SEQ ID NO: 3925), CAGgugcagg (SEQ ID NO: 1364), GUGgugagcu (SEQ ID NO: 2381), AAGguaacau (SEQ ID NO: 135), CUGgugaugg (SEQ ID NO: 3926), AUGguaaaug (SEQ ID NO: 882), CCGgugagca (SEQ ID NO: 3927), AAGguaaacc (SEQ ID NO: 124), AAGguacugg (SEQ ID NO: 3928), GCGgucagga (SEQ ID NO: 3929), CUGgucaggg (SEQ ID NO: 3930), AAAguacguu (SEQ ID NO: 3931), AGAguagguu (SEQ ID NO: 688), AGGguaagcu (SEQ ID NO: 3932), AUUgugagua (SEQ ID NO: 1009), CCGgccacca (SEQ ID NO: 3933), GAGguaacuu (SEQ ID NO: 1881), GAGguaugaa (SEQ ID NO: 1956), CAGgucagac (SEQ ID NO: 1276), UAGgcgugug (SEQ ID NO: 2462), AGGguaaguu (SEQ ID NO: 743), CAGgcaugag (SEQ ID NO: 1111), CAGguaacgu (SEQ ID NO: 1133), CAGgegagca (SEQ ID NO: 3934), UAGguauggu (SEQ ID NO: 2550), AGAguaggau (SEQ ID NO: 3935), CUGguuucaa (SEQ ID NO: 3936), GAGguaaacu (SEQ ID NO: 3937), CAGgcaugca (SEQ ID NO: 1112), UUGguaaucu (SEQ ID NO: 3938), AGGgcagaau (SEQ ID NO: 3939), AUGguaaaac (SEQ ID NO: 877), GCUgcaggug (SEQ ID NO: 3940), GAAgcacgug (SEQ ID NO: 3941), CAUguaaaca (SEQ ID NO: 3942), UGGguaagau (SEQ ID NO: 2835), AGGguagcua (SEQ ID NO: 3943), AGGguggggu (SEQ ID NO: 800), CCUguaaguu (SEQ ID NO: 1600), UGAgugaguu (SEQ ID NO: 2801), GGAguaugua (SEQ ID NO: 3944), CAGgugaccu (SEQ ID NO: 1328), AAAguacgga (SEQ ID NO: 3945), GAGguacaga (SEQ ID NO: 1906), GAUguaggua (SEQ ID NO: 2125), GGGguaauug (SEQ ID NO: 3946), UAGguggguu (SEQ ID NO: 2617), GUGguacgua (SEQ ID NO: 3947), AAGguacagc (SEQ ID NO: 3948), GAGgugaaga (SEQ ID NO: 3949), GGGguaagca (SEQ ID NO: 2246), UGAguagguc (SEQ ID NO: 3950), GGGguaaguu (SEQ ID NO: 2253), AUUgugaguu (SEQ ID NO: 1011), UCAguaagac (SEQ ID NO: 3951), AGUgugagcu (SEQ ID NO: 834), AAGgcaaaac (SEQ ID NO: 3952), CUGgugaguc (SEQ ID NO: 1760), AAGgucucug (SEQ ID NO: 310), GAGgcugugc (SEQ ID NO: 3953), AGAgugagac (SEQ ID NO: 700), GAGgugaugu (SEQ ID NO: 2033), AGAguauggu (SEQ ID NO: 3954), UGGguggguc (SEQ ID NO: 2884), GCUgcugagc (SEQ ID NO: 3955), CAGguagcug (SEQ ID NO: 1210), UAGgucagaa (SEQ ID NO: 3956), CCGguaggug (SEQ ID NO: 3957), GCAguaugau (SEQ ID NO: 3958), CAGguuucag (SEQ ID NO: 3959), GAGguuugcc (SEQ ID NO: 3960), GGGguggggg (SEQ ID NO: 3961), AAGguacaua (SEQ ID NO: 179), UGGguguguu (SEQ ID NO: 2890), AGAguaaggc (SEQ ID NO: 666), GCGguuagug (SEQ ID NO: 3962), AAGgugacuu (SEQ ID NO: 334), AUGguaagau (SEQ ID NO: 892), AUGguaguug (SEQ ID NO: 3963), CAUguaagac (SEQ ID NO: 3964), CUGguaugua (SEQ ID NO: 1736), UUCguaagga (SEQ ID NO: 3965), GAAguaugac (SEQ ID NO: 3966), CGGguaauuc (SEQ ID NO: 1627), UGGguaacuu (SEQ ID NO: 2831), CAGgugccua (SEQ ID NO: 1372), CAUguagggc (SEQ ID NO: 3967), ACCgucagga (SEQ ID NO: 3968), CGUguucgau (SEQ ID NO: 3969), GAGgcaggac (SEQ ID NO: 3970), UAGguaauau (SEQ ID NO: 2496), UCGguauacu (SEQ ID NO: 3971), UAGguugugc (SEQ ID NO: 3972), CCGgugaguc (SEQ ID NO: 3973), CAGgugccaa (SEQ ID NO: 1368), CAGgugaugc (SEQ ID NO: 1352), AAGgugagga (SEQ ID NO: 343), GUGgugaggg (SEQ ID NO: 3974), UGGgucagua (SEQ ID NO: 3975), GAGgucaggg (SEQ ID NO: 1985), UAGguacgua (SEQ ID NO: 2511), GAGgcaagag (SEQ ID NO: 1857), CCUguuggua (SEQ ID NO: 3976), GAGguaucca (SEQ ID NO: 3977), UAAguaagcu (SEQ ID NO: 2419), AAGgucaguu (SEQ ID NO: 296), AAAguuaaag (SEQ ID NO: 3978), GAGgugcuau (SEQ ID NO: 3979), ACGguaaguu (SEQ ID NO: 581), CUGgugaggg (SEQ ID NO: 1757), GAGguuaugu (SEQ ID NO: 2091), CUUgugugca (SEQ ID NO: 3980), UGAgcugggg (SEQ ID NO: 3981), AAGguauagu (SEQ ID NO: 3982), UAGguaaaac (SEQ ID NO: 2464), GGGgugaggu (SEQ ID NO: 3983), GAGgcaagca (SEQ ID NO: 3984), GGAguaacgu (SEQ ID NO: 3985), AGAguaagua (SEQ ID NO: 3986), AAAguaagua (SEQ ID NO: 21), GAGgcaacca (SEQ ID NO: 3987), UGUguaaguu (SEQ ID NO: 2909), UAGgugaggc (SEQ ID NO: 2594), ACAguaagaa (SEQ ID NO: 544), UGAguaagug (SEQ ID NO: 2774), CAAgucagua (SEQ ID NO: 1057), AGGguaaaug (SEQ ID NO: 3988), AAGguaugca (SEQ ID NO: 257), GCUgugcgug (SEQ ID NO: 3989), GAGguucgcc (SEQ ID NO: 3990), AAGgcuugca (SEQ ID NO: 3991), CAGgcaagug (SEQ ID NO: 1104), AUAguaaguc (SEQ ID NO: 3992), UUGguaggua (SEQ ID NO: 2978), GCAgcaggua (SEQ ID NO: 3993), AAGguauauc (SEQ ID NO: 243), AGCguaagcc (SEQ ID NO: 3994), CUGguucgaa (SEQ ID NO: 3995), ACGgugggug (SEQ ID NO: 612), CUGgucauug (SEQ ID NO: 3996), CAGgucagga (SEQ ID NO: 1280), CAAgugagac (SEQ ID NO: 1062), GAGguacugg (SEQ ID NO: 1919), GAGguguagu (SEQ ID NO: 3997), GAGguguccu (SEQ ID NO: 3998), CAGgugcgua (SEQ ID NO: 1380), AGUgcccuga (SEQ ID NO: 3999), AUGgugaguc (SEQ ID NO: 962), UGUgugugua (SEQ ID NO: 4000), CAGguaugcu (SEQ ID NO: 1254), CUGguacagu (SEQ ID NO: 4001), UUGguacgua (SEQ ID NO: 4002), UCUguacgua (SEQ ID NO: 4003), UAAguaauuc (SEQ ID NO: 4004), CACguaugug (SEQ ID NO: 4005), CAGgcaagua (SEQ ID NO: 1103), UCGgugagug (SEQ ID NO: 4006), GGUgugaguc (SEQ ID NO: 2315), UCUguaagcu (SEQ ID NO: 2743), AAGguucaga (SEQ ID NO: 4007), AGGguacuuc (SEQ ID NO: 4008), GCGgcagguu (SEQ ID NO: 4009), GAGgcccgug (SEQ ID NO: 4010), CAGguauaaa (SEQ ID NO: 4011), AUGgucaagu (SEQ ID NO: 4012), AAGgugagua (SEQ ID NO: 347), GUGguuuguu (SEQ ID NO: 4013), AGAgugagga (SEQ ID NO: 4014), GAGguaugac (SEQ ID NO: 1957), UAGgcgugag (SEQ ID NO: 4015), AAGguacucc (SEQ ID NO: 4016), UGAgugagga (SEQ ID NO: 2798), GAGguaugau (SEQ ID NO: 4017), GGGgucggua (SEQ ID NO: 4018), ACGguaugca (SEQ ID NO: 4019), CAGguaccac (SEQ ID NO: 1171), UAAguaccug (SEQ ID NO: 4020), AGGgugggcu (SEQ ID NO: 4021), CUGgucuguu (SEQ ID NO: 4022), UAGgucagag (SEQ ID NO: 4023), AAGguguguu (SEQ ID NO: 406), CUGgucagug (SEQ ID NO: 4024), AAGgugggac (SEQ ID NO: 4025), GUGguaguag (SEQ ID NO: 4026), CUAguuuagg (SEQ ID NO: 4027), CCCgccccau (SEQ ID NO: 4028), GCUguacugc (SEQ ID NO: 4029), GAGguaauau (SEQ ID NO: 1897), UAGguuggug (SEQ ID NO: 4030), AAGguccaac (SEQ ID NO: 4031), UAGgugagga (SEQ ID NO: 2593), GUGguaaguu (SEQ ID NO: 2354), AGUgugagag (SEQ ID NO: 831), AAUguacaug (SEQ ID NO: 497), UUGgcaggug (SEQ ID NO: 4032), UAGguuauug (SEQ ID NO: 4033), CAGguacuga (SEQ ID NO: 1191), GCGguggguc (SEQ ID NO: 4034), UGUguaagau (SEQ ID NO: 4035), GAGgugagua (SEQ ID NO: 2025), GCAgccccgg (SEQ ID NO: 4036), CAGgugcuaa (SEQ ID NO: 4037), AGUguaagag (SEQ ID NO: 815), CAGguacauc (SEQ ID NO: 4038), CAGgugggac (SEQ ID NO: 1403), AGGguaaaua (SEQ ID NO: 727), UAAguaauua (SEQ ID NO: 4039), CAGguaaccg (SEQ ID NO: 1132), AAGguuugca (SEQ ID NO: 461), UAGgugguuu (SEQ ID NO: 4040), CAGgugaccg (SEQ ID NO: 1327), UGUguaagcu (SEQ ID NO: 4041), GGAgugaguc (SEQ ID NO: 2227), AGGguaggag (SEQ ID NO: 752), AGGgugggug (SEQ ID NO: 802), AAGgucugag (SEQ ID NO: 313), GAUguaauau (SEQ ID NO: 4042), GGGguaauua (SEQ ID NO: 4043), UAGguaggua (SEQ ID NO: 2524), GAGgcaagua (SEQ ID NO: 1858), GAGguaagga (SEQ ID NO: 1889), UAGguacuac (SEQ ID NO: 4044), UCGgugggug (SEQ ID NO: 4045), AAGgugugga (SEQ ID NO: 401), CAGgucugcc (SEQ ID NO: 1305), UAAgugagcc (SEQ ID NO: 4046), GAAguaaguu (SEQ ID NO: 1820), GAAguaagcc (SEQ ID NO: 1815), UAGgugcgac (SEQ ID NO: 4047), GAGguauggc (SEQ ID NO: 4048), GCAguaagaa (SEQ ID NO: 2145), CAGgugugga (SEQ ID NO: 1438), UUGguaacgu (SEQ ID NO: 4049), GCUguaaaaa (SEQ ID NO: 4050), UUGguuagua (SEQ ID NO: 4051), AUAguaaggg (SEQ ID NO: 4052), UUGguacuag (SEQ ID NO: 4053), CGGgcagccg (SEQ ID NO: 4054), CAGgugcugg (SEQ ID NO: 1389), UAUgugaguu (SEQ ID NO: 2673), CAGgucuggg (SEQ ID NO: 4055), UAAguaagaa (SEQ ID NO: 2415), AAGguuauua (SEQ ID NO: 4056), AGAguaaagc (SEQ ID NO: 4057), AGAgugugag (SEQ ID NO: 4058), UAGgugcgag (SEQ ID NO: 4059), CAAguaaacg (SEQ ID NO: 4060), AAGguacgua (SEQ ID NO: 4061), CUGgugagua (SEQ ID NO: 1759), CCAguaugua (SEQ ID NO: 4062), UUGgugagug (SEQ ID NO: 3006), UGAguaagua (SEQ ID NO: 2772), GAGguuagca (SEQ ID NO: 4063), GUGguaagcc (SEQ ID NO: 4064), CUGguauggc (SEQ ID NO: 1734), AAAguaacac (SEQ ID NO: 8), CAGguacuaa (SEQ ID NO: 1186), UCUguaaguu (SEQ ID NO: 2747), GAGgugaggg (SEQ ID NO: 2024), ACUgugggua (SEQ ID NO: 647), GAUguuugug (SEQ ID NO: 4065), CAGgugucaa (SEQ ID NO: 4066), CAGgucacca (SEQ ID NO: 4067), CCGgugagua (SEQ ID NO: 4068), UUGguaaaua (SEQ ID NO: 4069), CAGguggggg (SEQ ID NO: 1411), ACUgcaggug (SEQ ID NO: 4070), UAGguauguu (SEQ ID NO: 2554), GGAgcaagug (SEQ ID NO: 4071), UCGgugccuc (SEQ ID NO: 4072), CAAguaacuu (SEQ ID NO: 4073), GAGguaacca (SEQ ID NO: 1879), CAGguaauau (SEQ ID NO: 1151), GGAguaagaa (SEQ ID NO: 4074), GAGguaccuu (SEQ ID NO: 1914), AGGguaagga (SEQ ID NO: 737), CCUgugaguc (SEQ ID NO: 1609), GAGguaaugg (SEQ ID NO: 1900), AUGguguguc (SEQ ID NO: 4075), GGGgugagua (SEQ ID NO: 4076), AGGgucaggu (SEQ ID NO: 4077), UGGguaaggg (SEQ ID NO: 2839), AGGguagguu (SEQ ID NO: 759), AUAgugaguu (SEQ ID NO: 4078), CCCguaggcu (SEQ ID NO: 4079), ACAguaugua (SEQ ID NO: 553), GACgugugua (SEQ ID NO: 4080), GCGgugagga (SEQ ID NO: 4081), CAGgugaccc (SEQ ID NO: 1326), UAAguuuagu (SEQ ID NO: 4082), ACAguugagu (SEQ ID NO: 570), CGGgugaggg (SEQ ID NO: 1639), CAGguggauu (SEQ ID NO: 1398), CGGguagagg (SEQ ID NO: 4083), UAGgugcgug (SEQ ID NO: 2608), GGGguaagaa (SEQ ID NO: 2243), GAGguggggu (SEQ ID NO: 4084), CACguggguu (SEQ ID NO: 4085), ACGguaauug (SEQ ID NO: 4086), AGAgugaguc (SEQ ID NO: 705), UUGgcuccaa (SEQ ID NO: 4087), AAGgugaugc (SEQ ID NO: 355), AAGguugguc (SEQ ID NO: 448), AGCguaaguu (SEQ ID NO: 4088), AUUguaugua (SEQ ID NO: 1006), UCAguuaagu (SEQ ID NO: 4089), CAAguacgug (SEQ ID NO: 4090), CAGgugcgug (SEQ ID NO: 1382), CAGguaggua (SEQ ID NO: 1220), AUGguggggu (SEQ ID NO: 4091), AUGgugaguu (SEQ ID NO: 964), CAGguaauca (SEQ ID NO: 4092), AAGguagggu (SEQ ID NO: 226), CAGgccaagg (SEQ ID NO: 4093), GUGgugagag (SEQ ID NO: 4094), AAGguuggug (SEQ ID NO: 449), CAGguacucu (SEQ ID NO: 1190), UAGgcaugug (SEQ ID NO: 4095), UUGguaccuu (SEQ ID NO: 4096), CUGgugugcc (SEQ ID NO: 4097), ACAguugcca (SEQ ID NO: 4098), UUGguaauau (SEQ ID NO: 4099), GAGgugcaug (SEQ ID NO: 4100), UUGguuugua (SEQ ID NO: 3028), UUGguaagug (SEQ ID NO: 2963), UGUgugugug (SEQ ID NO: 4101), GUGguuugua (SEQ ID NO: 2398), GCGguacaca (SEQ ID NO: 4102), AGAguaugcu (SEQ ID NO: 4103), UUUguaagua (SEQ ID NO: 3038), UCUgugcggg (SEQ ID NO: 4104), AAGgucagug (SEQ ID NO: 295), GAGguaggaa (SEQ ID NO: 1930), GCGguuagca (SEQ ID NO: 4105), AGGgugaggg (SEQ ID NO: 793), GAAgugagua (SEQ ID NO: 4106), CAGgugacag (SEQ ID NO: 4107), AAGgugauua (SEQ ID NO: 357), GAGgccagcc (SEQ ID NO: 4108), GAGgucuccu (SEQ ID NO: 4109), UAGguauuac (SEQ ID NO: 2556), CAUguaagag (SEQ ID NO: 1519), CUGguagggc (SEQ ID NO: 4110), GAAguaagua (SEQ ID NO: 1818), CGGguaagug (SEQ ID NO: 4111), CAGguaaucu (SEQ ID NO: 4112), GUGguaggua (SEQ ID NO: 4113), CAGgugggua (SEQ ID NO: 1413), AAGgccagug (SEQ ID NO: 4114), AAAgugaauc (SEQ ID NO: 4115), ACGguuacgu (SEQ ID NO: 4116), AUGguaggaa (SEQ ID NO: 917), CGGgugagac (SEQ ID NO: 4117), GAGguuggaa (SEQ ID NO: 2099), UGGgugagcc (SEQ ID NO: 2871), CCAgugagua (SEQ ID NO: 1564), CUAguacgag (SEQ ID NO: 4118), CAGguaugac (SEQ ID NO: 1248), GCUgugaggu (SEQ ID NO: 4119), CUGguaugaa (SEQ ID NO: 4120), GGUguacgac (SEQ ID NO: 4121), CUUgugagug (SEQ ID NO: 4122), GUGgugagca (SEQ ID NO: 2380), CUGguaacuu (SEQ ID NO: 1696), CAGguacuau (SEQ ID NO: 1188), AGGguaaggg (SEQ ID NO: 739), UUGguuaguu (SEQ ID NO: 3025), GGUguaagca (SEQ ID NO: 2302), UCGgugagga (SEQ ID NO: 4123), UGGguaaaca (SEQ ID NO: 4124), UCGguacgug (SEQ ID NO: 4125), UAGguagcag (SEQ ID NO: 4126), CUGguaaggc (SEQ ID NO: 1704), GUGguaagga (SEQ ID NO: 2349), UAAguaagca (SEQ ID NO: 2418), GAGguuccaa (SEQ ID NO: 4127), CUGguaugga (SEQ ID NO: 4128), GGGgugggua (SEQ ID NO: 2288), CAGguuuccc (SEQ ID NO: 4129), CAGgucucug (SEQ ID NO: 4130), GAGgugagga (SEQ ID NO: 2022), CUUguggguu (SEQ ID NO: 1805), AUGgugagac (SEQ ID NO: 953), CAGgugaagg (SEQ ID NO: 1319), GCGguagggg (SEQ ID NO: 4131), GUUguuuccc (SEQ ID NO: 4132), AAAgcaucca (SEQ ID NO: 4133), GUGguagguu (SEQ ID NO: 2367), AAGgugugaa (SEQ ID NO: 398), CAGguacagu (SEQ ID NO: 1167), AAGguaccaa (SEQ ID NO: 182), UUGguaauug (SEQ ID NO: 2969), AAGgugcuca (SEQ ID NO: 4134), AAGguucaac (SEQ ID NO: 4135), CAGguuuaca (SEQ ID NO: 4136), GCUguaagug (SEQ ID NO: 2195), AGGguauguc (SEQ ID NO: 769), GAGgucgggg (SEQ ID NO: 1996), AAGgugccug (SEQ ID NO: 363), AAGguaaaaa (SEQ ID NO: 119), GUGgugaguu (SEQ ID NO: 2385), UAGguaagaa (SEQ ID NO: 4137), AGGguauccu (SEQ ID NO: 4138), GUGguaauau (SEQ ID NO: 4139), UCUguaagua (SEQ ID NO: 2744), UGGguaugga (SEQ ID NO: 4140), AUGguaugga (SEQ ID NO: 935), GACgugagcc (SEQ ID NO: 1854), CUGguuuggc (SEQ ID NO: 4141), AUGguauauc (SEQ ID NO: 4142), AAAguaaacu (SEQ ID NO: 4143), AGCgugagug (SEQ ID NO: 721), CUGguauaga (SEQ ID NO: 4144), CAGgugggga (SEQ ID NO: 1409), AGAguauguu (SEQ ID NO: 696), UAGguacuug (SEQ ID NO: 4145), GCAguaggug (SEQ ID NO: 4146), AGUguauguc (SEQ ID NO: 4147), AAGguuaagc (SEQ ID NO: 413), CUGguggccu (SEQ ID NO: 4148), GAAgugaguc (SEQ ID NO: 1839), UUGguguaag (SEQ ID NO: 4149), CAGguaagaa (SEQ ID NO: 1138), CGGgucucgg (SEQ ID NO: 4150), GAGgugcaca (SEQ ID NO: 2035), CUCguuaguu (SEQ ID NO: 4151), AAGgugauca (SEQ ID NO: 352), UAUguaagaa (SEQ ID NO: 2649), GAGgugcuug (SEQ ID NO: 2047), CAGgugguca (SEQ ID NO: 4152), ACGguaaguc (SEQ ID NO: 4153), ACAguaaugu (SEQ ID NO: 4154), CCUguaaggu (SEQ ID NO: 4155), GAGguuaagu (SEQ ID NO: 4156), UCGguaugug (SEQ ID NO: 2725), UGGguauguu (SEQ ID NO: 2863), AAGguauuac (SEQ ID NO: 268), CAGgugaggg (SEQ ID NO: 1343), UUGguaaaca (SEQ ID NO: 4157), AAGguagugu (SEQ ID NO: 4158), GAGguguggc (SEQ ID NO: 4159), CAGguacgga (SEQ ID NO: 4160), AAGgucauca (SEQ ID NO: 4161), CAAguaggca (SEQ ID NO: 4162), CAGgugaaac (SEQ ID NO: 4163), CAGguacugc (SEQ ID NO: 1192), AAUgcaagug (SEQ ID NO: 4164), CAUguaauuc (SEQ ID NO: 4165), AAGguaugcu (SEQ ID NO: 259), CUGgugaguu (SEQ ID NO: 1762), CAGgugguuu (SEQ ID NO: 4166), UGUgugagua (SEQ ID NO: 2922), AAGgucggug (SEQ ID NO: 4167), AUGguaaauu (SEQ ID NO: 883), AGGguauuac (SEQ ID NO: 771), AGUguaugga (SEQ ID NO: 4168), AACguaagau (SEQ ID NO: 4169), GUGguaaggu (SEQ ID NO: 4170), ACUguuagua (SEQ ID NO: 4171), CAGguaucag (SEQ ID NO: 1239), AAGguuaguu (SEQ ID NO: 425), CUGgugagcu (SEQ ID NO: 1754), UUGgugagcu (SEQ ID NO: 4172), UGUguacgua (SEQ ID NO: 4173), GAGgucagcc (SEQ ID NO: 4174), GAGguagaau (SEQ ID NO: 4175), AAGguaugag (SEQ ID NO: 255), UAGguauuuc (SEQ ID NO: 2563), UGUguaacac (SEQ ID NO: 4176), AGUguaaggc (SEQ ID NO: 4177), GAGgucugcu (SEQ ID NO: 4178), AAGguuagca (SEQ ID NO: 418), CAGguaaaug (SEQ ID NO: 1127), AACguaagcu (SEQ ID NO: 4179), CAGgucugca (SEQ ID NO: 4180), CAGguauugu (SEQ ID NO: 1267), GUGguaauuc (SEQ ID NO: 2356), GAGguauaug (SEQ ID NO: 1951), GCCgugagcc (SEQ ID NO: 4181), GAGguaagag (SEQ ID NO: 1883), UGAguaugua (SEQ ID NO: 2787), CAGguaaggg (SEQ ID NO: 1145), GAGguaaauu (SEQ ID NO: 1876), CAGgcaacuu (SEQ ID NO: 4182), UGUguaaguc (SEQ ID NO: 2908), CAGgugcgcu (SEQ ID NO: 4183), CGGguaaacc (SEQ ID NO: 4184), CCGgucaguc (SEQ ID NO: 4185), UAGgugggg (SEQ ID NO: 4186), GCGgucaguu (SEQ ID NO: 4187), GGGguggguc (SEQ ID NO: 4188), AGCguaauag (SEQ ID NO: 4189), ACGgugaguc (SEQ ID NO: 4190), CUGguacuug (SEQ ID NO: 1722), CAGguuggua (SEQ ID NO: 4191), AGAguaugug (SEQ ID NO: 695), CUGgugggua (SEQ ID NO: 1771), GAGguggcuu (SEQ ID NO: 4192), AUAguauuga (SEQ ID NO: 4193), UGAgucgucc (SEQ ID NO: 4194), CAGgugcucu (SEQ ID NO: 4195), UACguaauau (SEQ ID NO: 4196), GCUguccuga (SEQ ID NO: 4197), CAGgcugcac (SEQ ID NO: 4198), CUGgugcgcu (SEQ ID NO: 1766), GCGguaagaa (SEQ ID NO: 4199), UAAguuacuu (SEQ ID NO: 4200), GAAgugagug (SEQ ID NO: 1840), UAGgcaaguc (SEQ ID NO: 2460), UAAguaaaua (SEQ ID NO: 4201), ACGgugagug (SEQ ID NO: 607), CAGguagguu (SEQ ID NO: 1223), GGGguauaac (SEQ ID NO: 4202), GUUgugaguu (SEQ ID NO: 2410), CAUgugagua (SEQ ID NO: 1539), GAGgugcauu (SEQ ID NO: 4203), AAGguuugua (SEQ ID NO: 466), UCGguaaugu (SEQ ID NO: 4204), CGAguaaggg (SEQ ID NO: 1616), GAGgcacgga (SEQ ID NO: 4205), AGGgugugga (SEQ ID NO: 4206), CAGguauggu (SEQ ID NO: 1257), AAGguagaaa (SEQ ID NO: 203), CAGgugccug (SEQ ID NO: 1373), UGGguauaug (SEQ ID NO: 4207), UGAgugagac (SEQ ID NO: 4208), UGGguaauuu (SEQ ID NO: 2847), AUGguaaaua (SEQ ID NO: 881), AAGgcaaagg (SEQ ID NO: 4209), AGUguuuguu (SEQ ID NO: 4210), AUGguauugg (SEQ ID NO: 4211), CUGgugaggc (SEQ ID NO: 1756), UUGguaaaau (SEQ ID NO: 2948), ACAgugaguu (SEQ ID NO: 563), CAGgugcugu (SEQ ID NO: 4212), GAGguuaaga (SEQ ID NO: 2080), AGAguaagaa (SEQ ID NO: 659), GAGgucegcg (SEQ ID NO: 4213), GUGgugagga (SEQ ID NO: 2382), CAGgugagcc (SEQ ID NO: 1338), CAGgugacau (SEQ ID NO: 1324), AUGgcaagcu (SEQ ID NO: 4214), UCGguaauau (SEQ ID NO: 4215), CAGgcaacaa (SEQ ID NO: 4216), GGGguaggga (SEQ ID NO: 2257), CUGgucucgc (SEQ ID NO: 4217), UAGguaacga (SEQ ID NO: 4218), CGGguaaggu (SEQ ID NO: 4219), UAGguaaugc (SEQ ID NO: 4220), CAGgcaagaa (SEQ ID NO: 1099), ACAguaggua (SEQ ID NO: 4221), CAAguaugag (SEQ ID NO: 1049), GCUguucgaa (SEQ ID NO: 4222), AAGguuaugc (SEQ ID NO: 4223), GAUgugaguu (SEQ ID NO: 2136), CAGguggaga (SEQ ID NO: 1396), AGAguuaguu (SEQ ID NO: 4224), UGAgugugeg (SEQ ID NO: 4225), GAGguacagc (SEQ ID NO: 1907), CAGguaagac (SEQ ID NO: 1139), CAUgugcuuu (SEQ ID NO: 4226), AGGguguguu (SEQ ID NO: 4227), ACAguuaagg (SEQ ID NO: 4228), ACAgugaggg (SEQ ID NO: 4229), GAUguauacc (SEQ ID NO: 4230), UUAguaagcu (SEQ ID NO: 4231), CAGguaagau (SEQ ID NO: 1141), AGAgcugcgu (SEQ ID NO: 4232), GAGgcaaguu (SEQ ID NO: 1860), GAAguaagug (SEQ ID NO: 1819), AAGgugaaaa (SEQ ID NO: 4233), AAGguaccua (SEQ ID NO: 4234), GAGguaucag (SEQ ID NO: 4235), AUGguaugua (SEQ ID NO: 4236), AAGguaugaa (SEQ ID NO: 253), UUGgugagcc (SEQ ID NO: 4237), AAGguuagga (SEQ ID NO: 420), AGGguaugua (SEQ ID NO: 768), CAGguaccga (SEQ ID NO: 4238), AGAguaaacu (SEQ ID NO: 4239), AAGgugcaua (SEQ ID NO: 4240), AAGguaaugu (SEQ ID NO: 167), CCGgugugug (SEQ ID NO: 4241), AGGguaaauu (SEQ ID NO: 729), GGGguuuggc (SEQ ID NO: 4242), CAGguacacg (SEQ ID NO: 1164), UUGguaacca (SEQ ID NO: 4243), GAGgucaggu (SEQ ID NO: 1986), UCUguuggua (SEQ ID NO: 4244), CAGguuaguu (SEQ ID NO: 1458), UUGguauguc (SEQ ID NO: 4245), AAGgugeguc (SEQ ID NO: 4246), AGGguaagaa (SEQ ID NO: 733), UUUguaagcc (SEQ ID NO: 4247), AAGgucaggu (SEQ ID NO: 292), CUGguaaacu (SEQ ID NO: 4248), UCGguaauuu (SEQ ID NO: 4249), CUGguaggcu (SEQ ID NO: 4250), GAGgucugua (SEQ ID NO: 4251), GAGguacuuu (SEQ ID NO: 1922), CUGguaaagg (SEQ ID NO: 4252), CGGgugugug (SEQ ID NO: 1650), CAGguguggu (SEQ ID NO: 4253), UCGguacguc (SEQ ID NO: 4254), CAGgugccag (SEQ ID NO: 4255), GGGgugagaa (SEQ ID NO: 2275), ACAgcuagua (SEQ ID NO: 4256), AAGguauagc (SEQ ID NO: 4257), CUGguaggag (SEQ ID NO: 4258), GCUguacgua (SEQ ID NO: 4259), AAGguaaagg (SEQ ID NO: 128), CAAgcacgag (SEQ ID NO: 4260), CUAguaagac (SEQ ID NO: 4261), CCCguaagcg (SEQ ID NO: 4262), CAAgugugag (SEQ ID NO: 1078), AUGguaaggg (SEQ ID NO: 897), AAGgugaggg (SEQ ID NO: 345), CAAguaggua (SEQ ID NO: 1041), GGUguugcug (SEQ ID NO: 2321), GAGguacugu (SEQ ID NO: 1920), UAGguaagau (SEQ ID NO: 2484), CAGgugcgaa (SEQ ID NO: 1374), GAGguccagg (SEQ ID NO: 4263), UUGguauaca (SEQ ID NO: 2982), GGAgugagua (SEQ ID NO: 2226), GAGgugagau (SEQ ID NO: 2017), AAGguggggc (SEQ ID NO: 4264), CAGguaaacg (SEQ ID NO: 4265), UCGguaacuu (SEQ ID NO: 4266), CAGguaaauu (SEQ ID NO: 1128), GAGgugcgca (SEQ ID NO: 4267), ACUgugagua (SEQ ID NO: 643), ACGgugugac (SEQ ID NO: 4268), GUGguaaguc (SEQ ID NO: 2352), CAGguaggca (SEQ ID NO: 1215), CAGgucagca (SEQ ID NO: 1277), GUGguaugug (SEQ ID NO: 4269), AAAguaucug (SEQ ID NO: 4270), CGGguaugua (SEQ ID NO: 4271), AAGguaauaa (SEQ ID NO: 157), GAGgugggga (SEQ ID NO: 2060), GCUguaggug (SEQ ID NO: 2197), GAAgugaguu (SEQ ID NO: 1841), AAAguauuua (SEQ ID NO: 4272), UAUguaagua (SEQ ID NO: 2653), ACGguaugag (SEQ ID NO: 4273), CUGgugagug (SEQ ID NO: 1761), AGAguaaaau (SEQ ID NO: 4274), GCUguauggc (SEQ ID NO: 4275), AUGguaaacc (SEQ ID NO: 879), GCAguaauaa (SEQ ID NO: 4276), UAAguauuua (SEQ ID NO: 4277), AAUgucagug (SEQ ID NO: 515), AUUgcaggag (SEQ ID NO: 4278), CCGguaagaa (SEQ ID NO: 4279), AAGgcaaguu (SEQ ID NO: 101), GAGguuuguc (SEQ ID NO: 4280), AAGguaacug (SEQ ID NO: 139), AAAguaugag (SEQ ID NO: 4281), GAUguuagua (SEQ ID NO: 4282), CAGguggguc (SEQ ID NO: 1414), AAGguaccga (SEQ ID NO: 4283), CCAguaauua (SEQ ID NO: 4284), GUGguaugcg (SEQ ID NO: 4285), AUGgugcgcu (SEQ ID NO: 4286), CAGgucuaug (SEQ ID NO: 4287), AAGguauuua (SEQ ID NO: 274), CUAguaagau (SEQ ID NO: 4288), AGAguaauuu (SEQ ID NO: 675), GAGguaacgu (SEQ ID NO: 4289), AAGguagcca (SEQ ID NO: 212), CUGgucccgg (SEQ ID NO: 4290), GAGguccuuc (SEQ ID NO: 4291), ACGgucaccc (SEQ ID NO: 4292), AAGguaauac (SEQ ID NO: 158), CAGgugcaug (SEQ ID NO: 1367), AUGguaauag (SEQ ID NO: 4293), UUUguaacac (SEQ ID NO: 4294), UGGguaugau (SEQ ID NO: 4295), CAGgcccccc (SEQ ID NO: 4296), AGAguaguaa (SEQ ID NO: 4297), AGUguaagaa (SEQ ID NO: 814), GAAguauguu (SEQ ID NO: 1833), CAGgugugca (SEQ ID NO: 1434), UUGgugaggg (SEQ ID NO: 3003), UGGguugguu (SEQ ID NO: 4298), CAGguacgua (SEQ ID NO: 1184), GAGgugcggc (SEQ ID NO: 4299), UCUguacggg (SEQ ID NO: 4300), CGGgugcgug (SEQ ID NO: 4301), UACguaagug (SEQ ID NO: 2455), CAUguaagga (SEQ ID NO: 4302), CAGgugacgg (SEQ ID NO: 1329), GAUguaugcu (SEQ ID NO: 4303), UCUgcaauuc (SEQ ID NO: 4304), UGAguaaggc (SEQ ID NO: 2770), GAGguauauu (SEQ ID NO: 1952), AGAgugaguu (SEQ ID NO: 707), AAGguaagcu (SEQ ID NO: 148), UAGgugaagu (SEQ ID NO: 2580), CAGguuagua (SEQ ID NO: 1455), UAUguaagug (SEQ ID NO: 2655), UUGguggggg (SEQ ID NO: 4305), UGAgcucaaa (SEQ ID NO: 4306), UCGguaugua (SEQ ID NO: 4307), UAAguaugcc (SEQ ID NO: 4308), AAUguaagua (SEQ ID NO: 489), CAGguuugca (SEQ ID NO: 4309), ACGgugagag (SEQ ID NO: 4310), CAGguguuuu (SEQ ID NO: 4311), GUGgugagcc (SEQ ID NO: 4312), AGGguacaua (SEQ ID NO: 4313), UAGguaaccc (SEQ ID NO: 4314), GUGgucagua (SEQ ID NO: 4315), CUGgugagcc (SEQ ID NO: 4316), CAGgugcuua (SEQ ID NO: 1390), AUAgucguga (SEQ ID NO: 4317), AUAgugagug (SEQ ID NO: 862), GAGgucaaaa (SEQ ID NO: 4318), CGUguagcuu (SEQ ID NO: 4319), CAGguguuug (SEQ ID NO: 4320), CAGguuggac (SEQ ID NO: 4321), CAGguaagcu (SEQ ID NO: 4322), AGGgucagaa (SEQ ID NO: 4323), CACguauguc (SEQ ID NO: 4324), CACgugagug (SEQ ID NO: 1098), GGGguacgga (SEQ ID NO: 4325), AAGgcaggac (SEQ ID NO: 4326), GAGgugaagc (SEQ ID NO: 4327), GAGguuugaa (SEQ ID NO: 4328), CAGguaagug (SEQ ID NO: 1148), CAGguaacca (SEQ ID NO: 1131), CAGguacucc (SEQ ID NO: 1189), AAGgugcuuu (SEQ ID NO: 371), GAGguaaaua (SEQ ID NO: 1873), GAGgcaggug (SEQ ID NO: 4329), GAGguucgga (SEQ ID NO: 4330), CAGguauuug (SEQ ID NO: 1270), CAGguaaaua (SEQ ID NO: 1125), CAGgugaugu (SEQ ID NO: 1354), CAGgugauac (SEQ ID NO: 4331), GAGgugaggc (SEQ ID NO: 2023), AGGguggggg (SEQ ID NO: 4332), UAAguaaguu (SEQ ID NO: 2425), UGGgugaaca (SEQ ID NO: 4333), UAGguacugc (SEQ ID NO: 4334), CAGgcuccug (SEQ ID NO: 4335), AGGguaggca (SEQ ID NO: 753), CAGgugcccg (SEQ ID NO: 1371), GAGguacauc (SEQ ID NO: 4336), AGGgugugug (SEQ ID NO: 804), AAGguaguaa (SEQ ID NO: 231), UGGguaugag (SEQ ID NO: 2859), GGGgugugug (SEQ ID NO: 2294), CUAguaggug (SEQ ID NO: 4337), GAGgcaagga (SEQ ID NO: 4338), AAGgcaagac (SEQ ID NO: 4339), AAAgugeggu (SEQ ID NO: 4340), AAGguugguu (SEQ ID NO: 450), GAGguuaaug (SEQ ID NO: 4341), UUGgugaguc (SEQ ID NO: 3005), UCGguuagcu (SEQ ID NO: 2738), GCAguaagca (SEQ ID NO: 4342), AAGgcaagca (SEQ ID NO: 4343), ACAguaagcu (SEQ ID NO: 4344), GAGguaacag (SEQ ID NO: 1878), AAAguacgua (SEQ ID NO: 4345), GAGguaauac (SEQ ID NO: 1896), UUGguaggug (SEQ ID NO: 2980), CUGguuaguc (SEQ ID NO: 4346), GAGgugacgc (SEQ ID NO: 4347), ACAguaagga (SEQ ID NO: 4348), AAUguacuua (SEQ ID NO: 4349), GGGguacagu (SEQ ID NO: 4350), CGUguaugug (SEQ ID NO: 4351), UCCguagguu (SEQ ID NO: 4352), GAGguggucg (SEQ ID NO: 4353), UCAgugaguc (SEQ ID NO: 4354), AAAguaagca (SEQ ID NO: 15), GAGgucuggu (SEQ ID NO: 1999), GAGguaauua (SEQ ID NO: 4355), GUAguaagua (SEQ ID NO: 2323), AAGgugggga (SEQ ID NO: 382), UCUgugagca (SEQ ID NO: 4356), GAAguucgug (SEQ ID NO: 4357), ACGgugaggc (SEQ ID NO: 4358), UCAgugagua (SEQ ID NO: 2699), UAGguaguug (SEQ ID NO: 4359), GGUgucuggg (SEQ ID NO: 4360), GGGguaagug (SEQ ID NO: 2252), GAGguggguu (SEQ ID NO: 2066), UGUgugaguu (SEQ ID NO: 4361), CAUguaagua (SEQ ID NO: 1522), AAGguaggug (SEQ ID NO: 229), AAUguaggag (SEQ ID NO: 4362), GAGgcacguc (SEQ ID NO: 4363), CAAguacauu (SEQ ID NO: 4364), UUGguacaga (SEQ ID NO: 4365), GAGguaguag (SEQ ID NO: 1941), AAAgugaggg (SEQ ID NO: 57), UUGgucagug (SEQ ID NO: 4366), AGGgugaguc (SEQ ID NO: 796), CAGgugaaca (SEQ ID NO: 1317), GGUgugggcc (SEQ ID NO: 4367), CGGgugagcu (SEQ ID NO: 4368), GGGgugaguc (SEQ ID NO: 2283), ACAgugagag (SEQ ID NO: 4369), AGGgugaggu (SEQ ID NO: 794), GCUguaaguc (SEQ ID NO: 2194), AUAguagguu (SEQ ID NO: 4370), CAGgcaugug (SEQ ID NO: 1114), AAGguaaguu (SEQ ID NO: 156), CAGguccgug (SEQ ID NO: 4371), GAGgcaggua (SEQ ID NO: 4372), AUGguggaag (SEQ ID NO: 4373), AUGgugggg (SEQ ID NO: 4374), GAGgugagaa (SEQ ID NO: 2014), AGUgugagca (SEQ ID NO: 832), UUGguaagua (SEQ ID NO: 2962), CAAguaagca (SEQ ID NO: 4375), GGUgugagcu (SEQ ID NO: 2313), CCCgugggua (SEQ ID NO: 4376), CAGguagaau (SEQ ID NO: 4377), CAGgcugagc (SEQ ID NO: 4378), CUGguggccc (SEQ ID NO: 4379), UGAguaagag (SEQ ID NO: 4380), CACguuagcu (SEQ ID NO: 4381), AAGgugaguc (SEQ ID NO: 348), AAGguagcuc (SEQ ID NO: 4382), UCGgugaguu (SEQ ID NO: 4383), GAGgcccuuc (SEQ ID NO: 4384), CAGguuaugc (SEQ ID NO: 4385), CCUguaagcu (SEQ ID NO: 4386), CAGgucuccu (SEQ ID NO: 4387), UAGguaggcu (SEQ ID NO: 4388), GGGguagggg (SEQ ID NO: 4389), AAGguaguga (SEQ ID NO: 4390), GAGguuguug (SEQ ID NO: 4391), CAGguugguu (SEQ ID NO: 1489), AAAguaagcc (SEQ ID NO: 16), ACAgugagug (SEQ ID NO: 562), UGGgugugau (SEQ ID NO: 4392), CCCguaacua (SEQ ID NO: 4393), AAGguguugc (SEQ ID NO: 408), AAAgcuggug (SEQ ID NO: 4394), GAGguauagu (SEQ ID NO: 4395), ACGguaagag (SEQ ID NO: 4396), AUGguacggu (SEQ ID NO: 913), GAGgccaguu (SEQ ID NO: 4397), GAGguaugcg (SEQ ID NO: 1960), UCGgugggag (SEQ ID NO: 4398), AAGguggaua (SEQ ID NO: 372), CCAguguggc (SEQ ID NO: 4399), AGGguaagug (SEQ ID NO: 742), UCUguagguc (SEQ ID NO: 4400), CAGgcaagga (SEQ ID NO: 1102), CGGguaauuu (SEQ ID NO: 1628), AUUgugaguc (SEQ ID NO: 1010), CAGguaaacc (SEQ ID NO: 1121), AAGgucaauu (SEQ ID NO: 4401), AAGgugaaua (SEQ ID NO: 327), GUCguaagaa (SEQ ID NO: 4402), GCGguaaguc (SEQ ID NO: 4403), CUGguagagc (SEQ ID NO: 4404), GAGgucgguc (SEQ ID NO: 4405), CAGguaaaca (SEQ ID NO: 1120), AAGgcaagga (SEQ ID NO: 98), CAGgucgucu (SEQ ID NO: 4406), GGGguagggc (SEQ ID NO: 4407), CUGguacuaa (SEQ ID NO: 1721), GAGguagcug (SEQ ID NO: 1929), CUUgucagcu (SEQ ID NO: 4408), UAGguaaggc (SEQ ID NO: 2489), CUGguauuac (SEQ ID NO: 4409), UAAguacguc (SEQ ID NO: 4410), AAGguaagcc (SEQ ID NO: 146), ACGgugaaag (SEQ ID NO: 4411), CCAgccaaua (SEQ ID NO: 4412), CAGguuuguc (SEQ ID NO: 4413), AAGguauaau (SEQ ID NO: 239), AAGgucuuag (SEQ ID NO: 4414), AGGgugagcu (SEQ ID NO: 791), AAGguuaggg (SEQ ID NO: 4415), CGGguaaauu (SEQ ID NO: 4416), CAGguaacgg (SEQ ID NO: 4417), AGAgugugua (SEQ ID NO: 4418), ACAguaaguu (SEQ ID NO: 549), GAUguaauuu (SEQ ID NO: 4419), GAGguaggga (SEQ ID NO: 1934), UUGgcaagug (SEQ ID NO: 2945), AAAgugagga (SEQ ID NO: 4420), AAGguagugc (SEQ ID NO: 234), AGAguaauuc (SEQ ID NO: 674), GGAguaaaua (SEQ ID NO: 4421), GUGguaccca (SEQ ID NO: 4422), CAGguauugc (SEQ ID NO: 4423), GAUgugaggg (SEQ ID NO: 4424), CAAguaaauc (SEQ ID NO: 1017), CAGgugucuc (SEQ ID NO: 1428), AAGguaacag (SEQ ID NO: 4425), UUGguaaaag (SEQ ID NO: 4426), CAGguaucau (SEQ ID NO: 1240), ACGgugagac (SEQ ID NO: 4427), CUGguaugac (SEQ ID NO: 4428), CAGguucacu (SEQ ID NO: 4429), GAGgugauca (SEQ ID NO: 4430), AGUguaaguc (SEQ ID NO: 4431), AACguaagua (SEQ ID NO: 4432), AAAgugagug (SEQ ID NO: 60), GAGguacagg (SEQ ID NO: 4433), CAAguaauga (SEQ ID NO: 4434), GAUguaagga (SEQ ID NO: 4435), UCAguucccc (SEQ ID NO: 4436), GCGguaagga (SEQ ID NO: 4437), UAGguacuaa (SEQ ID NO: 4438), AAGgugaaag (SEQ ID NO: 321), ACUguaagug (SEQ ID NO: 4439), UGGguaugug (SEQ ID NO: 2862), AUGguaacag (SEQ ID NO: 884), CAGguagggu (SEQ ID NO: 1219), ACAguaagug (SEQ ID NO: 548), AAGgugcucc (SEQ ID NO: 366), AAGgugugcu (SEQ ID NO: 4440), AAGgugguga (SEQ ID NO: 4441), ACGgugegcc (SEQ ID NO: 4442), AAGguauugc (SEQ ID NO: 4443), GGGguaugug (SEQ ID NO: 2267), CAGgugggcu (SEQ ID NO: 1408), GAGguauguu (SEQ ID NO: 1968), AACgugaaua (SEQ ID NO: 4444), CAGguaaugg (SEQ ID NO: 1154), UAGguaugau (SEQ ID NO: 4445), CAGgcaggug (SEQ ID NO: 1108), GGGguugguc (SEQ ID NO: 4446), AAGguauggg (SEQ ID NO: 262), UAAgugaggc (SEQ ID NO: 4447), CAAgugaucg (SEQ ID NO: 4448), AAAguacggg (SEQ ID NO: 4449), AGAgcuacag (SEQ ID NO: 4450), GAGgugggaa (SEQ ID NO: 2054), CAGguacuuu (SEQ ID NO: 1195), GAGgugagag (SEQ ID NO: 2016), CAGguagguc (SEQ ID NO: 1221), UGGguacagc (SEQ ID NO: 4451), AAGgugucag (SEQ ID NO: 396), AAGgcaagaa (SEQ ID NO: 4452), GAGguaaaca (SEQ ID NO: 4453), AAGguaaagu (SEQ ID NO: 129), AAGguaguca (SEQ ID NO: 4454), CUGguauguc (SEQ ID NO: 4455), GAGguauggg (SEQ ID NO: 1963), AAGguauugu (SEQ ID NO: 273), CUGguacuga (SEQ ID NO: 4456), GAGguaagcu (SEQ ID NO: 1888), UGGgugggua (SEQ ID NO: 2883), CAGguucgug (SEQ ID NO: 4457), AAGguauggu (SEQ ID NO: 4458), CAGgugagca (SEQ ID NO: 1337), UGGguaau (SEQ ID NO: 2827), UGUguaggug (SEQ ID NO: 4459), UGUgugagcc (SEQ ID NO: 2921), CUGguaauau (SEQ ID NO: 4460), AAAguauguu (SEQ ID NO: 45), UGUguaagaa (SEQ ID NO: 2903), CUAgugagaa (SEQ ID NO: 4461), AGGguagguc (SEQ ID NO: 757), AAGgugggug (SEQ ID NO: 385), UCGguaagug (SEQ ID NO: 4462), AGUguaaaua (SEQ ID NO: 812), GAUguaagug (SEQ ID NO: 2122), AAGguuagug (SEQ ID NO: 424), UAGguaagca (SEQ ID NO: 2485), CAAgugagaa (SEQ ID NO: 1061), AGUguaagua (SEQ ID NO: 819), CAGgugaauc (SEQ ID NO: 1321), UGGgugagac (SEQ ID NO: 2868), AAGguagggc (SEQ ID NO: 224), CUGguuugug (SEQ ID NO: 1788), GCGguagggc (SEQ ID NO: 4463), GAGguaaucc (SEQ ID NO: 4464), AUUguaauaa (SEQ ID NO: 4465), CUGgugaaua (SEQ ID NO: 1748), AAGguuuaaa (SEQ ID NO: 4466), CCUguacugu (SEQ ID NO: 4467), GCGgugageg (SEQ ID NO: 4468), AAGguaaucc (SEQ ID NO: 162), UAUgugagua (SEQ ID NO: 2671), CCCgugagug (SEQ ID NO: 1573), CAGgugcaga (SEQ ID NO: 1363), CAGgucaguu (SEQ ID NO: 1284), CAGguaggcu (SEQ ID NO: 4469), AAAguaagug (SEQ ID NO: 23), UAGguugguc (SEQ ID NO: 4470), CAGguugccu (SEQ ID NO: 4471), AAGguaugga (SEQ ID NO: 260), GGUguggacg (SEQ ID NO: 4472), AAAgugagaa (SEQ ID NO: 51), AGGgugagag (SEQ ID NO: 788), GAUguggcau (SEQ ID NO: 4473), UCGguaaggu (SEQ ID NO: 4474), GAGgugcguc (SEQ ID NO: 4475), CGGgugaguc (SEQ ID NO: 4476), AAGguacggg (SEQ ID NO: 190), GAGguucuug (SEQ ID NO: 4477), AAGgugcuug (SEQ ID NO: 4478), UAGguaugua (SEQ ID NO: 2551), AUGgucagca (SEQ ID NO: 4479), CGGguacuca (SEQ ID NO: 4480), AGGgugagga (SEQ ID NO: 792), AUCgugagua (SEQ ID NO: 869), UCAguaagua (SEQ ID NO: 2689), UAGguaaaua (SEQ ID NO: 2469), AAGguaauug (SEQ ID NO: 170), GAAgucagug (SEQ ID NO: 1835), CAGguacaaa (SEQ ID NO: 1160), AAAguuaauc (SEQ ID NO: 4481), AGCgugageg (SEQ ID NO: 4482), CCGgcuggug (SEQ ID NO: 4483), AGUguaauuu (SEQ ID NO: 4484), UGAgccacuc (SEQ ID NO: 4485), GGGgucugua (SEQ ID NO: 4486), AUGgcauguc (SEQ ID NO: 4487), CGGguaaaga (SEQ ID NO: 4488), AGGguagcau (SEQ ID NO: 4489), CGGguaggag (SEQ ID NO: 1631), GAGguucgug (SEQ ID NO: 4490), UAAguuauuc (SEQ ID NO: 4491), UAUguaagau (SEQ ID NO: 2650), AAGguaguuu (SEQ ID NO: 237), CAGgugguau (SEQ ID NO: 4492), GUGguaauga (SEQ ID NO: 2355), AAGgugauuu (SEQ ID NO: 359), CAGgugaagu (SEQ ID NO: 4493), GUAguaauua (SEQ ID NO: 4494), AUGguuggug (SEQ ID NO: 4495), CCAguaagug (SEQ ID NO: 1557), UAGgugagag (SEQ ID NO: 2589), AUGgugaggc (SEQ ID NO: 959), AAAguuagug (SEQ ID NO: 72), AAGgugccuu (SEQ ID NO: 4496), UAGguaugag (SEQ ID NO: 2546), CAGgugugac (SEQ ID NO: 1431), CUGguggguu (SEQ ID NO: 1774), AUGguaagga (SEQ ID NO: 896), UCUguaagaa (SEQ ID NO: 2740), UCCgugaguu (SEQ ID NO: 4497), AAAgcaggua (SEQ ID NO: 4498), UAUgugagug (SEQ ID NO: 2672), CAGguggagg (SEQ ID NO: 4499), CAGguuagac (SEQ ID NO: 4500), AUAguaagac (SEQ ID NO: 846), AAGguguugu (SEQ ID NO: 4501), GAGgucugug (SEQ ID NO: 4502), AAGguaagau (SEQ ID NO: 144), CAUguaaguu (SEQ ID NO: 1524), CUGguaauua (SEQ ID NO: 4503), CAGguaggcg (SEQ ID NO: 4504), AGAguaaguc (SEQ ID NO: 669), UGGgugagga (SEQ ID NO: 2872), AAUguaggua (SEQ ID NO: 4505), UAGguuagca (SEQ ID NO: 4506), GGGguaggua (SEQ ID NO: 2258), GAGguauugc (SEQ ID NO: 4507), AUUguacaca (SEQ ID NO: 4508), GAAguaggua (SEQ ID NO: 4509), GGAguaagcu (SEQ ID NO: 2212), UAGguaugug (SEQ ID NO: 2553), GAGgugaaua (SEQ ID NO: 2007), GAGgugggau (SEQ ID NO: 2056), AAGguaaucu (SEQ ID NO: 163), GGUgugaguu (SEQ ID NO: 4510), AACgugaguu (SEQ ID NO: 4511), GAGguaaccg (SEQ ID NO: 4512), UAGguaagga (SEQ ID NO: 2488), AUUguaagaa (SEQ ID NO: 4513), UGGgugagca (SEQ ID NO: 2870), AAGguaaggc (SEQ ID NO: 150), CCAguaucgu (SEQ ID NO: 4514), CCGgugggug (SEQ ID NO: 4515), GAGguagugu (SEQ ID NO: 4516), ACGgugggaa (SEQ ID NO: 4517), GAGgugaccu (SEQ ID NO: 2011), CACguaugua (SEQ ID NO: 4518), AGGgugggga (SEQ ID NO: 799), AAUguaaguc (SEQ ID NO: 490), AAAguuaagu (SEQ ID NO: 70), CAUgugagug (SEQ ID NO: 1541), AGAguauguc (SEQ ID NO: 694), GCGguaugac (SEQ ID NO: 4519), CGGgugaguu (SEQ ID NO: 1643), CCGguauuuu (SEQ ID NO: 4520), GAGguagaac (SEQ ID NO: 4521), UAGguaugaa (SEQ ID NO: 2545), CAGgcgcgug (SEQ ID NO: 4522), CAAguaaguc (SEQ ID NO: 1027), AGUguaagau (SEQ ID NO: 816), AAGguucuac (SEQ ID NO: 4523), CCAguaagua (SEQ ID NO: 1555), GAGguagcag (SEQ ID NO: 4524), CAGgucuguu (SEQ ID NO: 1312), CAGguacaau (SEQ ID NO: 1162), CCGguaaaga (SEQ ID NO: 1574), UAAgugcugu (SEQ ID NO: 4525), AGGgugagaa (SEQ ID NO: 786), CUCguaaggu (SEQ ID NO: 4526), CAGgucagcu (SEQ ID NO: 4527), CAGguaaggc (SEQ ID NO: 1144), AGGgugcagg (SEQ ID NO: 4528), GAGgugaaac (SEQ ID NO: 4529), AGGguaagua (SEQ ID NO: 740), AAUguaugcc (SEQ ID NO: 4530), AAGguaagca (SEQ ID NO: 145), ACGguacggu (SEQ ID NO: 587), AAGguaauga (SEQ ID NO: 164), UCUgcucaau (SEQ ID NO: 4531), ACGguaaugu (SEQ ID NO: 4532), AAGguaguug (SEQ ID NO: 4533), ACGguaagug (SEQ ID NO: 580), CAGgugauga (SEQ ID NO: 4534), GAGguaacac (SEQ ID NO: 4535), GAGguaggua (SEQ ID NO: 1937), CAGguaccuu (SEQ ID NO: 1179), CAGguaauaa (SEQ ID NO: 1150), UUGgugggug (SEQ ID NO: 3016), CUGguaauga (SEQ ID NO: 1710), UAGguaaguc (SEQ ID NO: 2492), AGGgugugac (SEQ ID NO: 4536), GAGgcaauaa (SEQ ID NO: 4537), GUGguaaagc (SEQ ID NO: 4538), CUGgugggeg (SEQ ID NO: 4539), GAUguauguu (SEQ ID NO: 2128), AGGgugagac (SEQ ID NO: 787), UCGgucagca (SEQ ID NO: 4540), AUGgugauua (SEQ ID NO: 4541), CGAgugugua (SEQ ID NO: 4542), CAGguuggug (SEQ ID NO: 1488), AGCgcaagua (SEQ ID NO: 4543), UGGguacguu (SEQ ID NO: 4544), GAGguauuug (SEQ ID NO: 1974), AGUguacaua (SEQ ID NO: 4545), AUGguaagua (SEQ ID NO: 898), ACAguagguu (SEQ ID NO: 4546), AAGgugagag (SEQ ID NO: 337), UUGgugaagu (SEQ ID NO: 4547), AAAguaugua (SEQ ID NO: 43), UGGguaagga (SEQ ID NO: 4548), UAGgugccuu (SEQ ID NO: 4549), and CCUgugggug (SEQ ID NO: 4550).

Additional exemplary gene sequences and splice site sequences (e.g., 5′ splice site sequences) include UCCguaaguu (SEQ ID NO: 4551), GUGguaaacg (SEQ ID NO: 4552), CGGgugcggu (SEQ ID NO: 4553), CAUguacuuc (SEQ ID NO: 4554), AGAguaaagg (SEQ ID NO: 4555), CGCgugagua (SEQ ID NO: 4556), AGAgugggca (SEQ ID NO: 4557), AGAguaagcc (SEQ ID NO: 4558), AGAguaaaca (SEQ ID NO: 4559), GUGguuauga (SEQ ID NO: 4560), AGGguaauaa (SEQ ID NO: 4561), UGAguaagac (SEQ ID NO: 4562), AGAguuuguu (SEQ ID NO: 4563), CGGgucugca (SEQ ID NO: 4564), CAGguaaguc (SEQ ID NO: 4565), AAGguagaau (SEQ ID NO: 4566), CAGgucccuc (SEQ ID NO: 4567), AGAguaaugg (SEQ ID NO: 4568), GAGgucuaag (SEQ ID NO: 4569), AGAguagagu (SEQ ID NO: 4570), AUGgucagua (SEQ ID NO: 4571), GAGgccuggg (SEQ ID NO: 4572), AAGguguggc (SEQ ID NO: 4573), AGAgugaucu (SEQ ID NO: 4574), AAGguaucca (SEQ ID NO: 4575), UUCguaagua (SEQ ID NO: 4576), UAAgugggug (SEQ ID NO: 4577), GCCgugaacg (SEQ ID NO: 4578), GAGguugugg (SEQ ID NO: 4579), UAUguaugca (SEQ ID NO: 4580), UGUguaacaa (SEQ ID NO: 4581), AGGguauuag (SEQ ID NO: 4582), UGAguauauc (SEQ ID NO: 4583), AGAguuugug (SEQ ID NO: 4584), GAGgucgcug (SEQ ID NO: 4585), GAGgucaucg (SEQ ID NO: 4586), ACGguaaagc (SEQ ID NO: 4587), UGAguacuug (SEQ ID NO: 4588), CGAgucgccg (SEQ ID NO: 4589), CUGguacguc (SEQ ID NO: 4590), AGGguauugc (SEQ ID NO: 4591), GAAgugaaug (SEQ ID NO: 4592), CAGaugaguc (SEQ ID NO: 4593), UGGguauugg (SEQ ID NO: 4594), UGAguaaaga (SEQ ID NO: 4595), GUGguuccug (SEQ ID NO: 4596), UGAgcaagua (SEQ ID NO: 4597), UAUguaagag (SEQ ID NO: 4598), AAGgucuugc (SEQ ID NO: 4599), AAAgcaugug (SEQ ID NO: 4600), AGAguacagu (SEQ ID NO: 4601), GUGguaaucc (SEQ ID NO: 4602), CAGguagagg (SEQ ID NO: 4603), AAGguacaac (SEQ ID NO: 4604), UGGgcagcau (SEQ ID NO: 4605), CCGgucauca (SEQ ID NO: 4606), CCGguuugua (SEQ ID NO: 4607), UGAguaaggg (SEQ ID NO: 4608), GAAguaugua (SEQ ID NO: 4609), GGGguagcuc (SEQ ID NO: 4610), GCUguacaua (SEQ ID NO: 4611), CUGgucucuu (SEQ ID NO: 4612), GUGguaaaug (SEQ ID NO: 4613), AUCguaagug (SEQ ID NO: 4614), GAGgcaugua (SEQ ID NO: 4615), AAGgucuccc (SEQ ID NO: 4616), UGGgugcguu (SEQ ID NO: 4617), UGUguagguu (SEQ ID NO: 4618), GAAgugagca (SEQ ID NO: 4619), GGUguaauuu (SEQ ID NO: 4620), CUGgugaaau (SEQ ID NO: 4621), AUCguaaguc (SEQ ID NO: 4622), AGAguaaucc (SEQ ID NO: 4623), GGAguagguc (SEQ ID NO: 4624), GAGguaccaa (SEQ ID NO: 4625), CUUguaggug (SEQ ID NO: 4626), AAGguauaag (SEQ ID NO: 4627), AGAguuggua (SEQ ID NO: 4628), AUGguuugug (SEQ ID NO: 4629), UGGgucagau (SEQ ID NO: 4630), AGAguaggac (SEQ ID NO: 4631), AGAguagugu (SEQ ID NO: 4632), AGAguaggag (SEQ ID NO: 4633), CAGgucucua (SEQ ID NO: 4634), AAGguggaug (SEQ ID NO: 4635), UGGguaucaa (SEQ ID NO: 4636), GAUguaugga (SEQ ID NO: 4637), AAGguguuuc (SEQ ID NO: 4638), GCAguguaaa (SEQ ID NO: 4639), UUAguaugua (SEQ ID NO: 4640), UCUguaugca (SEQ ID NO: 4641), AAUguaaaau (SEQ ID NO: 4642), AGAguaaauu (SEQ ID NO: 4643), GGGguacuuu (SEQ ID NO: 4644), GAAguuugau (SEQ ID NO: 4645), AAAguagauu (SEQ ID NO: 4646), UGUguagagu (SEQ ID NO: 4647), UGGguaageg (SEQ ID NO: 4648), CGGguucagg (SEQ ID NO: 4649), AGGguacgac (SEQ ID NO: 4650), UCGguaagaa (SEQ ID NO: 4651), AGGguuggca (SEQ ID NO: 4652), AAAguacagu (SEQ ID NO: 4653), UAAguuaagg (SEQ ID NO: 4654), AUGguaaugu (SEQ ID NO: 4655), GUGguuuuac (SEQ ID NO: 4656), AGAguaacaa (SEQ ID NO: 4657), AAGguagccc (SEQ ID NO: 4658), GCGgugaggc (SEQ ID NO: 4659), AUGguucagc (SEQ ID NO: 4660), AAGguacuua (SEQ ID NO: 4661), AAGguccgug (SEQ ID NO: 4662), UAGguaagcg (SEQ ID NO: 4663), AUGguaccuu (SEQ ID NO: 4664), GCCguggugg (SEQ ID NO: 4665), CUGgugcguc (SEQ ID NO: 4666), CAGguggaaa (SEQ ID NO: 4667), AAAgucugua (SEQ ID NO: 4668), GAGguaaccc (SEQ ID NO: 4669), AGAguauggg (SEQ ID NO: 4670), UAUgccccug (SEQ ID NO: 4671), AAGgugccag (SEQ ID NO: 4672), ACGgugeggc (SEQ ID NO: 4673), AGGguacuga (SEQ ID NO: 4674), AGAguaageg (SEQ ID NO: 4675), CUGgcaaggg (SEQ ID NO: 4676), CCAgugugug (SEQ ID NO: 4677), GAGguagacg (SEQ ID NO: 4678), CGGgugcggg (SEQ ID NO: 4679), GAUguaagcu (SEQ ID NO: 4680), AUUguauuua (SEQ ID NO: 4681), UGCgugagug (SEQ ID NO: 4682), CUGgucuaua (SEQ ID NO: 4683), GAGgugcuag (SEQ ID NO: 4684), GAGgugccau (SEQ ID NO: 4685), CAGguacguc (SEQ ID NO: 4686), GAGguucagc (SEQ ID NO: 4687), AACguaagaa (SEQ ID NO: 4688), AGAguaguac (SEQ ID NO: 4689), AAGguaacgg (SEQ ID NO: 4690), UAGgugugac (SEQ ID NO: 4691), CCGguaauag (SEQ ID NO: 4692), CAGguaccag (SEQ ID NO: 4693), UUUguaauug (SEQ ID NO: 4694), AAUguacgaa (SEQ ID NO: 4695), CAGguaauga (SEQ ID NO: 4696), AUCgucaagg (SEQ ID NO: 4697), CUGguagaug (SEQ ID NO: 4698), GGGgugcagu (SEQ ID NO: 4699), AGUgugagaa (SEQ ID NO: 4700), GGGguuuuau (SEQ ID NO: 4701), CCUguccccu (SEQ ID NO: 4702), AUUgugaagu (SEQ ID NO: 4703), AAGguaaacg (SEQ ID NO: 4704), UACgucgugg (SEQ ID NO: 4705), AAGgugccau (SEQ ID NO: 4706), GGGgucccag (SEQ ID NO: 4707), UAUguauggu (SEQ ID NO: 4708), CGGguaauua (SEQ ID NO: 4709), CGGguacucc (SEQ ID NO: 4710), CAGgugacuu (SEQ ID NO: 4711), AGUguggguu (SEQ ID NO: 4712), AGAguauggc (SEQ ID NO: 4713), AAGgccaaca (SEQ ID NO: 4714), AAAgcaagua (SEQ ID NO: 4715), UCAguagguc (SEQ ID NO: 4716), GUGguggegg (SEQ ID NO: 4717), CAUguauccu (SEQ ID NO: 4718), UCGgugagcc (SEQ ID NO: 4719), AUAguugggu (SEQ ID NO: 4720), AAUguuagcu (SEQ ID NO: 4721), AUGgugaaug (SEQ ID NO: 4722), CGGguaaugu (SEQ ID NO: 4723), UCUguaggug (SEQ ID NO: 4724), CCGgugaggc (SEQ ID NO: 4725), UGAguccacu (SEQ ID NO: 4726), CUAguaagag (SEQ ID NO: 4727), CGGguggggc (SEQ ID NO: 4728), CGAguaagca (SEQ ID NO: 4729), UGUgccaauu (SEQ ID NO: 4730), UCGguaagcc (SEQ ID NO: 4731), UAUguaggug (SEQ ID NO: 4732), UUGgugggcc (SEQ ID NO: 4733), GAGgcugggc (SEQ ID NO: 4734), AGAguaacuu (SEQ ID NO: 4735), ACGguagguc (SEQ ID NO: 4736), CAGgcccaga (SEQ ID NO: 4737), CCGguggguu (SEQ ID NO: 4738), AAGgugacgg (SEQ ID NO: 4739), GGGguacagc (SEQ ID NO: 4740), CAUguaaguc (SEQ ID NO: 4741), AUUgugagaa (SEQ ID NO: 4742), UGUguaagga (SEQ ID NO: 4743), UUUguaagau (SEQ ID NO: 4744), AGGgucauuu (SEQ ID NO: 4745), UGGguuuguu (SEQ ID NO: 4746), CGAguaagcc (SEQ ID NO: 4747), GUGgugugua (SEQ ID NO: 4748), AUGguauaac (SEQ ID NO: 4749), UGGguacgua (SEQ ID NO: 4750), AAAguagagu (SEQ ID NO: 4751), UCGguaacug (SEQ ID NO: 4752), AGAguaauga (SEQ ID NO: 4753), AUGguggguc (SEQ ID NO: 4754), AGAguaauau (SEQ ID NO: 4755), CAGguacugg (SEQ ID NO: 4756), UAAgucaguu (SEQ ID NO: 4757), GCGguagaga (SEQ ID NO: 4758), AAGgugaugg (SEQ ID NO: 4759), ACAguauguu (SEQ ID NO: 4760), GAUguacguc (SEQ ID NO: 4761), UAGguuucuc (SEQ ID NO: 4762), GAGgcauggg (SEQ ID NO: 4763), AUAgcuaagu (SEQ ID NO: 4764), GUAgucugua (SEQ ID NO: 4765), AAGgugaacg (SEQ ID NO: 4766), GUGguggucg (SEQ ID NO: 4767), GAGguugauc (SEQ ID NO: 4768), UGAguggguu (SEQ ID NO: 4769), ACUguacgug (SEQ ID NO: 4770), CUGgugacug (SEQ ID NO: 4771), CAAguuaagc (SEQ ID NO: 4772), GAGguaccca (SEQ ID NO: 4773), AACguaacuu (SEQ ID NO: 4774), CAGguuacua (SEQ ID NO: 4775), AGAguuaguc (SEQ ID NO: 4776), UGGgcacguc (SEQ ID NO: 4777), AGUguauggu (SEQ ID NO: 4778), AAGguugcaa (SEQ ID NO: 4779), CAGguuguua (SEQ ID NO: 4780), AAGgcauccc (SEQ ID NO: 4781), GAUguaaggc (SEQ ID NO: 4782), AGGguacggg (SEQ ID NO: 4783), GAGgucaaag (SEQ ID NO: 4784), CAAgugagcg (SEQ ID NO: 4785), AGAguaaucu (SEQ ID NO: 4786), UCGguagcug (SEQ ID NO: 4787), AAAguaguag (SEQ ID NO: 4788), CAGguucguc (SEQ ID NO: 4789), CGUguaugaa (SEQ ID NO: 4790), AGUguaaaaa (SEQ ID NO: 4791), AAGgucucac (SEQ ID NO: 4792), UAGguggagc (SEQ ID NO: 4793), UGAguaggug (SEQ ID NO: 4794), AGAguaugcc (SEQ ID NO: 4795), GAGguugcau (SEQ ID NO: 4796), CAAguaagag (SEQ ID NO: 4797), UCUgugugcc (SEQ ID NO: 4798), GAGgugaugc (SEQ ID NO: 4799), GGGgugauaa (SEQ ID NO: 4800), CCCgugagcc (SEQ ID NO: 4801), AGAguaacug (SEQ ID NO: 4802), GCGguaagua (SEQ ID NO: 4803), AGAguacauc (SEQ ID NO: 4804), UCGgucuggg (SEQ ID NO: 4805), UAAguaucuc (SEQ ID NO: 4806), GGCguagguu (SEQ ID NO: 4807), AGAguacgcc (SEQ ID NO: 4808), GAUgucuucu (SEQ ID NO: 4809), AGGgcaaggu (SEQ ID NO: 4810), CGAguaugau (SEQ ID NO: 4811), AUGguagagu (SEQ ID NO: 4812), CAAguacgag (SEQ ID NO: 4813), UCGguaugau (SEQ ID NO: 4814), CCGguguguu (SEQ ID NO: 4815), AGGgucugug (SEQ ID NO: 4816), GGAguaggcu (SEQ ID NO: 4817), AAGgucuaug (SEQ ID NO: 4818), GCAgugcgug (SEQ ID NO: 4819), UGGgugagaa (SEQ ID NO: 4820), AGGguaaagu (SEQ ID NO: 4821), GAGguaggac (SEQ ID NO: 4822), CUAguaagca (SEQ ID NO: 4823), UUAguaggcu (SEQ ID NO: 4824), CUGgugggau (SEQ ID NO: 4825), CUGguuagua (SEQ ID NO: 4826), AAGguacgug (SEQ ID NO: 4827), CGGgugagau (SEQ ID NO: 4828), AAGgugcaug (SEQ ID NO: 4829), AAUgugggcu (SEQ ID NO: 4830), CAGguugacu (SEQ ID NO: 4831), CAGguuacag (SEQ ID NO: 4832), GCGguaacau (SEQ ID NO: 4833), AUUgucaguc (SEQ ID NO: 4834), CAAguauaca (SEQ ID NO: 4835), GAUguccgcc (SEQ ID NO: 4836), AAGgugegga (SEQ ID NO: 4837), AACguaagag (SEQ ID NO: 4838), UGGguuggua (SEQ ID NO: 4839), CAAguguaag (SEQ ID NO: 4840), GUGguaacgu (SEQ ID NO: 4841), CUGgugauca (SEQ ID NO: 4842), AGGguggggc (SEQ ID NO: 4843), UCGguaaaga (SEQ ID NO: 4844), CAGguacacc (SEQ ID NO: 4845), CGGguaaggg (SEQ ID NO: 4846), CAAguuugcu (SEQ ID NO: 4847), ACAgugcgug (SEQ ID NO: 4848), UUGguauggg (SEQ ID NO: 4849), GAGgcucauc (SEQ ID NO: 4850), CUGguaauag (SEQ ID NO: 4851), AUGguggaua (SEQ ID NO: 4852), UCAgugaauu (SEQ ID NO: 4853), AAUguaauua (SEQ ID NO: 4854), GCAgucuaaa (SEQ ID NO: 4855), AAGguauucu (SEQ ID NO: 4856), GAGgucauca (SEQ ID NO: 4857), UGGguccaug (SEQ ID NO: 4858), AGAguuugua (SEQ ID NO: 4859), AGGguagacu (SEQ ID NO: 4860), AAGguaggac (SEQ ID NO: 4861), UGUguguuga (SEQ ID NO: 4862), UCAguacgug (SEQ ID NO: 4863), AUGgucucuc (SEQ ID NO: 4864), UGAguuagua (SEQ ID NO: 4865), UGAguaaagu (SEQ ID NO: 4866), GAGgugaccg (SEQ ID NO: 4867), GAGguauauc (SEQ ID NO: 4868), CAGgugccau (SEQ ID NO: 4869), AGAgugguga (SEQ ID NO: 4870), GUUguaagaa (SEQ ID NO: 4871), AGAguaaaua (SEQ ID NO: 4872), AGGgugaagg (SEQ ID NO: 4873), CUGguagauu (SEQ ID NO: 4874), GAGguucagg (SEQ ID NO: 4875), AGGgucuuca (SEQ ID NO: 4876), CUGguaaccu (SEQ ID NO: 4877), ACAguacuga (SEQ ID NO: 4878), AGAguggguc (SEQ ID NO: 4879), AUGguaugag (SEQ ID NO: 4880), AAGguuauau (SEQ ID NO: 4881), AGAguauagu (SEQ ID NO: 4882), AAAguaugaa (SEQ ID NO: 4883), UAGguggcua (SEQ ID NO: 4884), ACCguauggg (SEQ ID NO: 4885), AAAguauaau (SEQ ID NO: 4886), UUUguauggc (SEQ ID NO: 4887), GGGgucgcgu (SEQ ID NO: 4888), GUGgugguuu (SEQ ID NO: 4889), CAGguuugac (SEQ ID NO: 4890), GGAguaggcg (SEQ ID NO: 4891), GAGguacccu (SEQ ID NO: 4892), AUGgugugca (SEQ ID NO: 4893), GUGguuggug (SEQ ID NO: 4894), AAAguaugcu (SEQ ID NO: 4895), UAAguuacau (SEQ ID NO: 4896), ACAguaugag (SEQ ID NO: 4897), GGAguauguu (SEQ ID NO: 4898), UUUgugagaa (SEQ ID NO: 4899), AAUgugcguu (SEQ ID NO: 4900), CAGguagagu (SEQ ID NO: 4901), AUGguguuaa (SEQ ID NO: 4902), CAUgugcguc (SEQ ID NO: 4903), AUAguuggau (SEQ ID NO: 4904), GAGguacgua (SEQ ID NO: 4905), GUUgugagaa (SEQ ID NO: 4906), CAAguacauc (SEQ ID NO: 4907), GAGguaguuu (SEQ ID NO: 4908), ACUguacaga (SEQ ID NO: 4909), CCGguuguga (SEQ ID NO: 4910), UGGgucagug (SEQ ID NO: 4911), GUAguaagaa (SEQ ID NO: 4912), GACguacuuu (SEQ ID NO: 4913), AGAgucaguc (SEQ ID NO: 4914), UAGguuaguu (SEQ ID NO: 4915), AGGgcagcag (SEQ ID NO: 4916), AAGguccuac (SEQ ID NO: 4917), AAUguaauug (SEQ ID NO: 4918), CAGgugcggg (SEQ ID NO: 4919), CUGguaaugg (SEQ ID NO: 4920), CAAguagccc (SEQ ID NO: 4921), GAAgucaguu (SEQ ID NO: 4922), ACAguaauug (SEQ ID NO: 4923), UUAguuagua (SEQ ID NO: 4924), CCUguauuuu (SEQ ID NO: 4925), AUCguaagaa (SEQ ID NO: 4926), CCAgugagca (SEQ ID NO: 4927), GAAguaaggc (SEQ ID NO: 4928), UGAgugggua (SEQ ID NO: 4929), UCAgugguag (SEQ ID NO: 4930), UCUguacagg (SEQ ID NO: 4931), CGAgugagug (SEQ ID NO: 4932), UCCguaugug (SEQ ID NO: 4933), CAUgccguuu (SEQ ID NO: 4934), AAAgugacuu (SEQ ID NO: 4935), AGAguaggca (SEQ ID NO: 4936), GAAguaagag (SEQ ID NO: 4937), CAGgcagguu (SEQ ID NO: 4938), UUGguagagc (SEQ ID NO: 4939), AAGguggaaa (SEQ ID NO: 4940), GAGgcagguc (SEQ ID NO: 4941), AUGguacgac (SEQ ID NO: 4942), AGGguaggaa (SEQ ID NO: 4943), AGGguaggua (SEQ ID NO: 4944), UUGguaaggu (SEQ ID NO: 4945), AUGguacaga (SEQ ID NO: 4946), CAGguagagc (SEQ ID NO: 4947), UAGguaaggu (SEQ ID NO: 4948), GGGguuagag (SEQ ID NO: 4949), AAGguaucaa (SEQ ID NO: 4950), GAGguagccc (SEQ ID NO: 4951), CAGgugccuc (SEQ ID NO: 4952), GCAguaagag (SEQ ID NO: 4953), ACGguagagu (SEQ ID NO: 4954), UGGguaaugg (SEQ ID NO: 4955), CUGgucaguu (SEQ ID NO: 4956), GUGguacauu (SEQ ID NO: 4957), AAAguagguu (SEQ ID NO: 4958), AAGgccaaga (SEQ ID NO: 4959), CGGgugggca (SEQ ID NO: 4960), ACGguccggg (SEQ ID NO: 4961), CGAguaugag (SEQ ID NO: 4962), CUGguaugcc (SEQ ID NO: 4963), GAGguggaug (SEQ ID NO: 4964), CAGgccuuuc (SEQ ID NO: 4965), AAAguacauc (SEQ ID NO: 4966), AAAguaauca (SEQ ID NO: 4967), GAGguaacug (SEQ ID NO: 4968), CUGguaaaga (SEQ ID NO: 4969), CGUguaagca (SEQ ID NO: 4970), UGGgcaagua (SEQ ID NO: 4971), GCGguggcga (SEQ ID NO: 4972), GAGguggccg (SEQ ID NO: 4973), AUUgcaugca (SEQ ID NO: 4974), ACGgugacug (SEQ ID NO: 4975), CAGgucagau (SEQ ID NO: 4976), AGAguaacuc (SEQ ID NO: 4977), UGAguaacag (SEQ ID NO: 4978), AAGguacccg (SEQ ID NO: 4979), AGGguaggcu (SEQ ID NO: 4980), GGGgcaggac (SEQ ID NO: 4981), CCUguaagug (SEQ ID NO: 4982), AUUguaagug (SEQ ID NO: 4983), ACUguacgag (SEQ ID NO: 4984), GUAguagugu (SEQ ID NO: 4985), AGAguaugag (SEQ ID NO: 4986), UCAguguggg (SEQ ID NO: 4987), UGGguauaua (SEQ ID NO: 4988), UAGguagcua (SEQ ID NO: 4989), GGGguaaaga (SEQ ID NO: 4990), AGGguuacuu (SEQ ID NO: 4991), CAUguaaaug (SEQ ID NO: 4992), GGAguaguaa (SEQ ID NO: 4993), CAGgucaauc (SEQ ID NO: 4994), CGGguuagug (SEQ ID NO: 4995), UAGguacaug (SEQ ID NO: 4996), UAGguuaaga (SEQ ID NO: 4997), UGGguaccuu (SEQ ID NO: 4998), CGGguggaca (SEQ ID NO: 4999), CAGgucuuac (SEQ ID NO: 5000), AAGguggagc (SEQ ID NO: 5001), AUGguaacca (SEQ ID NO: 5002), UCGguaaguu (SEQ ID NO: 5003), UAUguacaaa (SEQ ID NO: 5004), AAUguagauu (SEQ ID NO: 5005), GUAgcuagua (SEQ ID NO: 5006), AAGguauugg (SEQ ID NO: 5007), GAGgucuuug (SEQ ID NO: 5008), GAAguucagg (SEQ ID NO: 5009), UGGguaucac (SEQ ID NO: 5010), AGAguacugg (SEQ ID NO: 5011), CAGguuaaug (SEQ ID NO: 5012), AGGguacgug (SEQ ID NO: 5013), AGGgcacagg (SEQ ID NO: 5014), CUGguuaguu (SEQ ID NO: 5015), UUGguacgag (SEQ ID NO: 5016), ACGgugauca (SEQ ID NO: 5017), CCUgugagag (SEQ ID NO: 5018), GAGgugaagu (SEQ ID NO: 5019), AAGguacauc (SEQ ID NO: 5020), UCUguaugug (SEQ ID NO: 5021), UUGguggaag (SEQ ID NO: 5022), UGGgcagguu (SEQ ID NO: 5023), GAAguggagc (SEQ ID NO: 5024), ACAguaagac (SEQ ID NO: 5025), CGGguaccaa (SEQ ID NO: 5026), CAAguacguc (SEQ ID NO: 5027), AGAgugaggg (SEQ ID NO: 5028), CGGguaagaa (SEQ ID NO: 5029), AAUguaggug (SEQ ID NO: 5030), AUCgugugcu (SEQ ID NO: 5031), UAGgucaugg (SEQ ID NO: 5032), CAGguuuuga (SEQ ID NO: 5033), AAGgcaugca (SEQ ID NO: 5034), GAGgugcugc (SEQ ID NO: 5035), AAGguuaaua (SEQ ID NO: 5036), CAGguucauc (SEQ ID NO: 5037), GCGguaggug (SEQ ID NO: 5038), GACgugagua (SEQ ID NO: 5039), CAGgucuacu (SEQ ID NO: 5040), UUGguaugag (SEQ ID NO: 5041), AGCgugggca (SEQ ID NO: 5042), AUGguaaggu (SEQ ID NO: 5043), AUGguaccuc (SEQ ID NO: 5044), UUGguauggu (SEQ ID NO: 5045), UAUguaugaa (SEQ ID NO: 5046), UGGguauggg (SEQ ID NO: 5047), GAUguaaaua (SEQ ID NO: 5048), CCGguaaguu (SEQ ID NO: 5049), GAGgucugaa (SEQ ID NO: 5050), GAGgugcgag (SEQ ID NO: 5051), CUGgucagcc (SEQ ID NO: 5052), CAGguuuugu (SEQ ID NO: 5053), CGGguggugu (SEQ ID NO: 5054), UAAguuagua (SEQ ID NO: 5055), UUUgugugug (SEQ ID NO: 5056), CAGguuaacc (SEQ ID NO: 5057), UUGguacuuu (SEQ ID NO: 5058), GCUguaaggc (SEQ ID NO: 5059), AGGguggcug (SEQ ID NO: 5060), GAUguaaaaa (SEQ ID NO: 5061), AAGgucaaaa (SEQ ID NO: 5062), CAGguagcgc (SEQ ID NO: 5063), CAGguuuggc (SEQ ID NO: 5064), GAGgugguuu (SEQ ID NO: 5065), CGGguaaaua (SEQ ID NO: 5066), CUGguucggu (SEQ ID NO: 5067), GGAgugagcc (SEQ ID NO: 5068), AAGgugegcg (SEQ ID NO: 5069), GAAguacauc (SEQ ID NO: 5070), AGUgucugua (SEQ ID NO: 5071), CCCgugagcu (SEQ ID NO: 5072), GAGguucaca (SEQ ID NO: 5073), CUAgugggua (SEQ ID NO: 5074), GAGguaacua (SEQ ID NO: 5075), UCGguauguc (SEQ ID NO: 5076), UAAguauuug (SEQ ID NO: 5077), CAGguaagcg (SEQ ID NO: 5078), GAGgugguaa (SEQ ID NO: 5079), CGAguaagag (SEQ ID NO: 5080), CCGguaagcu (SEQ ID NO: 5081), GAGgucuugu (SEQ ID NO: 5082), AAGguggguc (SEQ ID NO: 5083), CACguaagug (SEQ ID NO: 5084), AGUguaauga (SEQ ID NO: 5085), AAAgugugua (SEQ ID NO: 5086), GGAgugccaa (SEQ ID NO: 5087), CACgugaguu (SEQ ID NO: 5088), AAGguuggau (SEQ ID NO: 5089), UAUguaaaua (SEQ ID NO: 5090), CUGguaggaa (SEQ ID NO: 5091), UAUguaaacu (SEQ ID NO: 5092), AAUguauuuu (SEQ ID NO: 5093), CUGgcaagug (SEQ ID NO: 5094), UGUgugguau (SEQ ID NO: 5095), UAUguauguu (SEQ ID NO: 5096), UUGgugacuc (SEQ ID NO: 5097), GGAguaaggu (SEQ ID NO: 5098), AAGguagaug (SEQ ID NO: 5099), UGGguagggu (SEQ ID NO: 5100), AAUguaauuc (SEQ ID NO: 5101), GUGguauggc (SEQ ID NO: 5102), GGAguggguu (SEQ ID NO: 5103), AGGguaccac (SEQ ID NO: 5104), UAGgugacag (SEQ ID NO: 5105), ACAguaggca (SEQ ID NO: 5106), AUGguuugaa (SEQ ID NO: 5107), GCAguaacua (SEQ ID NO: 5108), CCGguaggua (SEQ ID NO: 5109), AGAguaggcc (SEQ ID NO: 5110), AAGguugaca (SEQ ID NO: 5111), CUGgugugua (SEQ ID NO: 5112), GAAgucuguc (SEQ ID NO: 5113), UGGgcucgga (SEQ ID NO: 5114), CAGguagccu (SEQ ID NO: 5115), AGAguaggua (SEQ ID NO: 5116), UAAguauguc (SEQ ID NO: 5117), CUGguauauc (SEQ ID NO: 5118), GAGguguguu (SEQ ID NO: 5119), AUGgugcaug (SEQ ID NO: 5120), AAGguacgcc (SEQ ID NO: 5121), UGAguaacua (SEQ ID NO: 5122), GAGgugacag (SEQ ID NO: 5123), GUUguccugu (SEQ ID NO: 5124), UUGgugucuu (SEQ ID NO: 5125), AAUgugaagg (SEQ ID NO: 5126), UUGguggaua (SEQ ID NO: 5127), UAGguguguu (SEQ ID NO: 5128), CUGgcaaguu (SEQ ID NO: 5129), GCAguaagau (SEQ ID NO: 5130), GCGguggaaa (SEQ ID NO: 5131), UGCguccagc (SEQ ID NO: 5132), AAAguggagu (SEQ ID NO: 5133), CGUgugagcc (SEQ ID NO: 5134), AGAguacugu (SEQ ID NO: 5135), CAGguauagc (SEQ ID NO: 5136), UACguaagga (SEQ ID NO: 5137), AAGgucuuua (SEQ ID NO: 5138), AAGguggucu (SEQ ID NO: 5139), GGGguaaauu (SEQ ID NO: 5140), UCAgugagga (SEQ ID NO: 5141), AGAguacguu (SEQ ID NO: 5142), GAGgucguca (SEQ ID NO: 5143), UAGguuugau (SEQ ID NO: 5144), CAUguaaacc (SEQ ID NO: 5145), AAGguggcac (SEQ ID NO: 5146), CAGguagaug (SEQ ID NO: 5147), AACguaaaag (SEQ ID NO: 5148), UAGgucucug (SEQ ID NO: 5149), AUAguaggug (SEQ ID NO: 5150), UAGgcaagag (SEQ ID NO: 5151), UAGgcacggc (SEQ ID NO: 5152), AAGgucuuca (SEQ ID NO: 5153), CCAguaugcu (SEQ ID NO: 5154), CAAgugaguu (SEQ ID NO: 5155), CAGgucucaa (SEQ ID NO: 5156), CAGguuacau (SEQ ID NO: 5157), GGAgugagca (SEQ ID NO: 5158), AGAguacgca (SEQ ID NO: 5159), CUGguguugg (SEQ ID NO: 5160), AAGguacuca (SEQ ID NO: 5161), CUAguaaggg (SEQ ID NO: 5162), AGAguaaaag (SEQ ID NO: 5163), AAGguaacga (SEQ ID NO: 5164), CUGguccccg (SEQ ID NO: 5165), UAAguauggg (SEQ ID NO: 5166), GAGgucgagc (SEQ ID NO: 5167), UUGguauaua (SEQ ID NO: 5168), AAAgucaagg (SEQ ID NO: 5169), AAGgucuagg (SEQ ID NO: 5170), CGAguagguc (SEQ ID NO: 5171), AGGguucguu (SEQ ID NO: 5172), GAGgcaggcc (SEQ ID NO: 5173), CUAguauuac (SEQ ID NO: 5174), ACGguaugug (SEQ ID NO: 5175), UAGgugguuc (SEQ ID NO: 5176), AGAguauaac (SEQ ID NO: 5177), UUGgugeguc (SEQ ID NO: 5178), ACCguuaucu (SEQ ID NO: 5179), CCAgugauga (SEQ ID NO: 5180), GAAguaugca (SEQ ID NO: 5181), GAAguauggc (SEQ ID NO: 5182), CCGguaggac (SEQ ID NO: 5183), AAUguaagca (SEQ ID NO: 5184), AGAguaauug (SEQ ID NO: 5185), AGGguugguu (SEQ ID NO: 5186), GUGguaggag (SEQ ID NO: 5187), AAGgcaguuu (SEQ ID NO: 5188), CAAguaagcc (SEQ ID NO: 5189), CUGgcaagua (SEQ ID NO: 5190), CAGgcaugau (SEQ ID NO: 5191), AGGguaauug (SEQ ID NO: 5192), GGGguaaccu (SEQ ID NO: 5193), AAAguaacua (SEQ ID NO: 5194), UAGgucugcc (SEQ ID NO: 5195), ACGguaugaa (SEQ ID NO: 5196), AGUguauggg (SEQ ID NO: 5197), UGGguuggca (SEQ ID NO: 5198), UAGguaaacu (SEQ ID NO: 5199), AGAgugggua (SEQ ID NO: 5200), AGAguauuug (SEQ ID NO: 5201), AGUguaggaa (SEQ ID NO: 5202), CUUguacgua (SEQ ID NO: 5203), GAUgugagau (SEQ ID NO: 5204), CAGgcagcca (SEQ ID NO: 5205), AAGgucacug (SEQ ID NO: 5206), AAGgucugac (SEQ ID NO: 5207), UAGguuccuu (SEQ ID NO: 5208), CUGgugcuuu (SEQ ID NO: 5209), UGAguuggug (SEQ ID NO: 5210), UUGgugggau (SEQ ID NO: 5211), UGAguagggu (SEQ ID NO: 5212), UCGgugaggu (SEQ ID NO: 5213), AAAguaaaga (SEQ ID NO: 5214), AAGgcaaguc (SEQ ID NO: 5215), CGGguaaagc (SEQ ID NO: 5216), AAAguuaguu (SEQ ID NO: 5217), UUAguaagca (SEQ ID NO: 5218), GAGgucacau (SEQ ID NO: 5219), UAAgugguau (SEQ ID NO: 5220), UAGgugcuuu (SEQ ID NO: 5221), GGAguaggca (SEQ ID NO: 5222), UGAguaagga (SEQ ID NO: 5223), CAGguggagc (SEQ ID NO: 5224), GAUguagaag (SEQ ID NO: 5225), AAUgccugcc (SEQ ID NO: 5226), AUGguaaggc (SEQ ID NO: 5227), UGGguaauau (SEQ ID NO: 5228), CUGguaccuc (SEQ ID NO: 5229), CACgugagcc (SEQ ID NO: 5230), UGAguuugug (SEQ ID NO: 5231), CCGguagugu (SEQ ID NO: 5232), AAAgugacaa (SEQ ID NO: 5233), GAAguggguu (SEQ ID NO: 5234), CAGgugcagc (SEQ ID NO: 5235), GAGgugggcc (SEQ ID NO: 5236), UAUgugcguc (SEQ ID NO: 5237), GGGguacugg (SEQ ID NO: 5238), CUGguagguu (SEQ ID NO: 5239), UUGgcauguu (SEQ ID NO: 5240), AAUguaauac (SEQ ID NO: 5241), UAGgccggug (SEQ ID NO: 5242), AGAgucagua (SEQ ID NO: 5243), UAAguaaauc (SEQ ID NO: 5244), CAGguuccuc (SEQ ID NO: 5245), UAGguacgau (SEQ ID NO: 5246), AGAguuagug (SEQ ID NO: 5247), GCAguaagug (SEQ ID NO: 5248), AGGgugguag (SEQ ID NO: 5249), GGAguaaugu (SEQ ID NO: 5250), GAUguaaguc (SEQ ID NO: 5251), CCAguuucgu (SEQ ID NO: 5252), AAGguucggg (SEQ ID NO: 5253), AUGguggagu (SEQ ID NO: 5254), AAGguaccgg (SEQ ID NO: 5255), GAAgugcgaa (SEQ ID NO: 5256), UGGgucaguu (SEQ ID NO: 5257), AAGguguaga (SEQ ID NO: 5258), UGGguaggcc (SEQ ID NO: 5259), CCAgugaguc (SEQ ID NO: 5260), AAGgucacuu (SEQ ID NO: 5261), AGCgugaggc (SEQ ID NO: 5262), UCCgugguaa (SEQ ID NO: 5263), AGAguacuua (SEQ ID NO: 5264), GGGgucagau (SEQ ID NO: 5265), AAGguggacc (SEQ ID NO: 5266), AGAgugageg (SEQ ID NO: 5267), AGAgucagau (SEQ ID NO: 5268), UAAguauuac (SEQ ID NO: 5269), AGAguauuuc (SEQ ID NO: 5270), AGAguucagc (SEQ ID NO: 5271), AUGgugaagu (SEQ ID NO: 5272), UAGgugaucc (SEQ ID NO: 5273), GGAguaagau (SEQ ID NO: 5274), UAGguaccaa (SEQ ID NO: 5275), AGAguugguc (SEQ ID NO: 5276), GAAgugagac (SEQ ID NO: 5277), AUCguagguu (SEQ ID NO: 5278), GAGguacgcu (SEQ ID NO: 5279), ACGguaaggg (SEQ ID NO: 5280), CAGgcauguc (SEQ ID NO: 5281), UUAguaagau (SEQ ID NO: 5282), UGAguagguu (SEQ ID NO: 5283), AGGguacgaa (SEQ ID NO: 5284), ACGguauguu (SEQ ID NO: 5285), AGGguacugu (SEQ ID NO: 5286), UUGguaugga (SEQ ID NO: 5287), UAAguaacug (SEQ ID NO: 5288), GCGgucagcc (SEQ ID NO: 5289), UUUgugaguc (SEQ ID NO: 5290), GUGgucagug (SEQ ID NO: 5291), CUGgucugua (SEQ ID NO: 5292), GAGguucuua (SEQ ID NO: 5293), AUGguacuga (SEQ ID NO: 5294), AAUgugcuuu (SEQ ID NO: 5295), AGGguggcgu (SEQ ID NO: 5296), CCGgcaggaa (SEQ ID NO: 5297), CAUguggguc (SEQ ID NO: 5298), UUGguuuguu (SEQ ID NO: 5299), CAGguucugu (SEQ ID NO: 5300), ACGguaagcg (SEQ ID NO: 5301), CUGgucagua (SEQ ID NO: 5302), UCAguaggcu (SEQ ID NO: 5303), UGAguaggac (SEQ ID NO: 5304), CAGguuuuaa (SEQ ID NO: 5305), GAGguguccc (SEQ ID NO: 5306), AGGguggguu (SEQ ID NO: 5307), GUGgugagac (SEQ ID NO: 5308), CACguaggga (SEQ ID NO: 5309), GUGguauuuu (SEQ ID NO: 5310), GAGauauccu (SEQ ID NO: 5311), AAGgugaaca (SEQ ID NO: 5312), UAAguagggc (SEQ ID NO: 5313), CUGgugcggg (SEQ ID NO: 5314), CUGgucaaua (SEQ ID NO: 5315), AGAguaaaaa (SEQ ID NO: 5316), AAGgugcagu (SEQ ID NO: 5317), CGGguaagca (SEQ ID NO: 5318), AAAgugagcc (SEQ ID NO: 5319), AUGguaauca (SEQ ID NO: 5320), GCAguacgug (SEQ ID NO: 5321), AUGguacaug (SEQ ID NO: 5322), AAGguuaaga (SEQ ID NO: 5323), CGGguaaaug (SEQ ID NO: 5324), GAGguucgca (SEQ ID NO: 5325), GAGgcucugg (SEQ ID NO: 5326), AUGgugggac (SEQ ID NO: 5327), AACgugguag (SEQ ID NO: 5328), AAGgugauag (SEQ ID NO: 5329), GGGguuugca (SEQ ID NO: 5330), CAUguaaggg (SEQ ID NO: 5331), UCAguugagu (SEQ ID NO: 5332), AAAgugcggc (SEQ ID NO: 5333), AGAgugagcc (SEQ ID NO: 5334), AUGgcaagaa (SEQ ID NO: 5335), ACAguaaggu (SEQ ID NO: 5336), AAGgucucua (SEQ ID NO: 5337), GUGguaaaaa (SEQ ID NO: 5338), AAAguaggug (SEQ ID NO: 5339), UAGgugcacu (SEQ ID NO: 5340), GUCgugguau (SEQ ID NO: 5341), CAGguauagg (SEQ ID NO: 5342), UGAgugagag (SEQ ID NO: 5343), ACUgugagcc (SEQ ID NO: 5344), AUCguuaguu (SEQ ID NO: 5345), UUUguaccaa (SEQ ID NO: 5346), UGGgugagau (SEQ ID NO: 5347), AGAgugagaa (SEQ ID NO: 5348), AGAguagggg (SEQ ID NO: 5349), AGGgcaagua (SEQ ID NO: 5350), CGGgucagua (SEQ ID NO: 5351), UUGguaugcc (SEQ ID NO: 5352), CGGguuagau (SEQ ID NO: 5353), GGGgugaagu (SEQ ID NO: 5354), CCCgugugaa (SEQ ID NO: 5355), GCAguuugga (SEQ ID NO: 5356), UGCguaagac (SEQ ID NO: 5357), AGAgucugua (SEQ ID NO: 5358), CACgugagca (SEQ ID NO: 5359), AGGguaaaag (SEQ ID NO: 5360), CAGgcugggu (SEQ ID NO: 5361), GAAgucuuca (SEQ ID NO: 5362), AAGgcaaaaa (SEQ ID NO: 5363), GUAguaaaua (SEQ ID NO: 5364), CUAgugagag (SEQ ID NO: 5365), GAAguuucug (SEQ ID NO: 5366), CCUguacgua (SEQ ID NO: 5367), GAGgugegcg (SEQ ID NO: 5368), AAGguguaaa (SEQ ID NO: 5369), CCAguauguu (SEQ ID NO: 5370), CCGgucagcu (SEQ ID NO: 5371), AUGguuccug (SEQ ID NO: 5372), CAAguuaaau (SEQ ID NO: 5373), AGAguaggcu (SEQ ID NO: 5374), AUGgugggca (SEQ ID NO: 5375), GGAguaagac (SEQ ID NO: 5376), AGGgucacga (SEQ ID NO: 5377), UAGgugauau (SEQ ID NO: 5378), GAAguaaguc (SEQ ID NO: 5379), CGGguaagau (SEQ ID NO: 5380), CAAguagcua (SEQ ID NO: 5381), UGAguaaaau (SEQ ID NO: 5382), GUCguacgug (SEQ ID NO: 5383), AUGguacgua (SEQ ID NO: 5384), CAGgucucgg (SEQ ID NO: 5385), GAGgcauguc (SEQ ID NO: 5386), AGAgugggau (SEQ ID NO: 5387), GUGguuagag (SEQ ID NO: 5388), UGGgugguga (SEQ ID NO: 5389), AAGguuaaac (SEQ ID NO: 5390), CUUguuagcu (SEQ ID NO: 5391), AAAguaggaa (SEQ ID NO: 5392), UAGguuguau (SEQ ID NO: 5393), AGGgugegcc (SEQ ID NO: 5394), AAGgugggcu (SEQ ID NO: 5395), UAAguaucug (SEQ ID NO: 5396), AAGguaacgu (SEQ ID NO: 5397), AUGguggggc (SEQ ID NO: 5398), CAAguacacg (SEQ ID NO: 5399), GGCguaagug (SEQ ID NO: 5400), AUAguaggac (SEQ ID NO: 5401), AGAgugaggu (SEQ ID NO: 5402), UUUguaaaaa (SEQ ID NO: 5403), GAAguuugua (SEQ ID NO: 5404), CUAguaaucu (SEQ ID NO: 5405), AAGguuuuua (SEQ ID NO: 5406), GAGgugcguu (SEQ ID NO: 5407), UAGgcgagua (SEQ ID NO: 5408), ACCgugagua (SEQ ID NO: 5409), CAGgucccga (SEQ ID NO: 5410), AUGguacugg (SEQ ID NO: 5411), UGAguucagu (SEQ ID NO: 5412), AAUguguggu (SEQ ID NO: 5413), UCCguugguu (SEQ ID NO: 5414), CAGgucagag (SEQ ID NO: 5415), CAGgucccua (SEQ ID NO: 5416), UAGguagacu (SEQ ID NO: 5417), CAAguuaagg (SEQ ID NO: 5418), GAGgugugcg (SEQ ID NO: 5419), GAAgcugccc (SEQ ID NO: 5420), CGAguacgug (SEQ ID NO: 5421), CGGguaggua (SEQ ID NO: 5422), UUGguauuga (SEQ ID NO: 5423), AUUguaugau (SEQ ID NO: 5424), UUGguaugaa (SEQ ID NO: 5425), GAGgugguca (SEQ ID NO: 5426), GCUguaugaa (SEQ ID NO: 5427), CAGguguugc (SEQ ID NO: 5428), CAGguaaaac (SEQ ID NO: 5429), AUAguaaggu (SEQ ID NO: 5430), CUGguuagag (SEQ ID NO: 5431), AGCgugugag (SEQ ID NO: 5432), AAGguuaucu (SEQ ID NO: 5433), CACgugagua (SEQ ID NO: 5434), AGGgucagua (SEQ ID NO: 5435), GAGguauaau (SEQ ID NO: 5436), CAGguuuuu (SEQ ID NO: 5437), AGGguggacu (SEQ ID NO: 5438), AUUguaauuc (SEQ ID NO: 5439), UUUguggguu (SEQ ID NO: 5440), AUGguacgug (SEQ ID NO: 5441), AAGguguucc (SEQ ID NO: 5442), CAGgugacgc (SEQ ID NO: 5443), GAGguacuaa (SEQ ID NO: 5444), ACAguucagu (SEQ ID NO: 5445), GAGgucacgg (SEQ ID NO: 5446), CAAguaaggc (SEQ ID NO: 5447), AAGguuuggg (SEQ ID NO: 5448), AAAgugggcu (SEQ ID NO: 5449), GCGguucuug (SEQ ID NO: 5450), GAGguggagc (SEQ ID NO: 5451), UGAgucagug (SEQ ID NO: 5452), CAGgucaagg (SEQ ID NO: 5453), AGUguaagcu (SEQ ID NO: 5454), GAGgcagaaa (SEQ ID NO: 5455), AAGgucacac (SEQ ID NO: 5456), GAAguagguu (SEQ ID NO: 5457), GUCguaaguu (SEQ ID NO: 5458), AGAguaugca (SEQ ID NO: 5459), CCUgugcaaa (SEQ ID NO: 5460), ACGgugaaaa (SEQ ID NO: 5461), CAGguacgaa (SEQ ID NO: 5462), CAUgugagga (SEQ ID NO: 5463), AGCgugagua (SEQ ID NO: 5464), GGUguguagg (SEQ ID NO: 5465), AACgugagcu (SEQ ID NO: 5466), GAGgugaacu (SEQ ID NO: 5467), AGAguucagu (SEQ ID NO: 5468), AACgugugua (SEQ ID NO: 5469), CAGguugugg (SEQ ID NO: 5470), AAGguacuag (SEQ ID NO: 5471), UCAgugaaaa (SEQ ID NO: 5472), AAUgucuggu (SEQ ID NO: 5473), ACGguaaaau (SEQ ID NO: 5474), CUGguguaag (SEQ ID NO: 5475), GAGgugcgaa (SEQ ID NO: 5476), AGGguuucuc (SEQ ID NO: 5477), CAGguagccc (SEQ ID NO: 5478), AUUguauugg (SEQ ID NO: 5479), AUGguacuua (SEQ ID NO: 5480), GAGgcccgac (SEQ ID NO: 5481), UCGguaagac (SEQ ID NO: 5482), CGGgcuguag (SEQ ID NO: 5483), UAUgugugug (SEQ ID NO: 5484), UAGguagaaa (SEQ ID NO: 5485), GUGgucauua (SEQ ID NO: 5486), UAGgugaaag (SEQ ID NO: 5487), ACUguaauuc (SEQ ID NO: 5488), GCAguacagg (SEQ ID NO: 5489), UCGgugaguc (SEQ ID NO: 5490), UAUguaggga (SEQ ID NO: 5491), AUGguauguc (SEQ ID NO: 5492), GUGgugugug (SEQ ID NO: 5493), CUGgugaccu (SEQ ID NO: 5494), AAUgugaaua (SEQ ID NO: 5495), UAGgucucac (SEQ ID NO: 5496), GAGguuauug (SEQ ID NO: 5497), UGAguaggcu (SEQ ID NO: 5498), CGGgcacgua (SEQ ID NO: 5499), GCAguaaaua (SEQ ID NO: 5500), CCGgugagag (SEQ ID NO: 5501), UAAguugguc (SEQ ID NO: 5502), CCGgugagcc (SEQ ID NO: 5503), AAGguuguca (SEQ ID NO: 5504), CUGguauuau (SEQ ID NO: 5505), GGGguauggg (SEQ ID NO: 5506), AAAgucagua (SEQ ID NO: 5507), UUUguaugua (SEQ ID NO: 5508), UAAguacugc (SEQ ID NO: 5509), CAGguaccaa (SEQ ID NO: 5510), GAAguucaga (SEQ ID NO: 5511), AUGgugcggu (SEQ ID NO: 5512), GUGgugaggu (SEQ ID NO: 5513), UGAguaagcc (SEQ ID NO: 5514), UAUguaaggg (SEQ ID NO: 5515), GUGguggaaa (SEQ ID NO: 5516), GAGgugauug (SEQ ID NO: 5517), GGAguuugua (SEQ ID NO: 5518), AAGgucacga (SEQ ID NO: 5519), GUGguagagg (SEQ ID NO: 5520), UAAguauauc (SEQ ID NO: 5521), AAGgugucca (SEQ ID NO: 5522), UAUgugguau (SEQ ID NO: 5523), GAGguacaau (SEQ ID NO: 5524), AAGguggggg (SEQ ID NO: 5525), GGAguaggug (SEQ ID NO: 5526), and UAGgugacuu (SEQ ID NO: 5527).

In some embodiments, the splice site sequence (e.g., 5′ splice site sequence) comprises AGA. In some embodiments, the splice site sequence (e.g., 5′ splice site sequence) comprises AAA. In some embodiments, the splice site sequence (e.g., 5′ splice site sequence) comprises AAC. In some embodiments, the splice site sequence (e.g., 5′ splice site sequence) comprises AAU. In some embodiments, the splice site sequence (e.g., 5′ splice site sequence) comprises AAG. In some embodiments, the splice site sequence (e.g., 5′ splice site sequence) comprises ACA. In some embodiments, the splice site sequence (e.g., 5′ splice site sequence) comprises AUA. In some embodiments, the splice site sequence (e.g., 5′ splice site sequence) comprises AUU. In some embodiments, the splice site sequence (e.g., 5′ splice site sequence) comprises AUG. In some embodiments, the splice site sequence (e.g., 5′ splice site sequence) comprises AUC. In some embodiments, the splice site sequence (e.g., 5′ splice site sequence) comprises CAA. In some embodiments, the splice site sequence (e.g., 5′ splice site sequence) comprises CAU. In some embodiments, the splice site sequence (e.g., 5′ splice site sequence) comprises CAC. In some embodiments, the splice site sequence (e.g., 5′ splice site sequence) comprises CAG. In some embodiments, the splice site sequence (e.g., 5′ splice site sequence) comprises GAA. In some embodiments, the splice site sequence (e.g., 5′ splice site sequence) comprises GAC. In some embodiments, the splice site sequence (e.g., 5′ splice site sequence) comprises GAU. In some embodiments, the splice site sequence (e.g., 5′ splice site sequence) comprises GAG. In some embodiments, the splice site sequence (e.g., 5′ splice site sequence) comprises GGA. In some embodiments, the splice site sequence (e.g., 5′ splice site sequence) comprises GCA. In some embodiments, the splice site sequence (e.g., 5′ splice site sequence) comprises GGG. In some embodiments, the splice site sequence (e.g., 5′ splice site sequence) comprises GGC. In some embodiments, the splice site sequence (e.g., 5′ splice site sequence) comprises GUU. In some embodiments, the splice site sequence (e.g., 5′ splice site sequence) comprises GGU. In some embodiments, the splice site sequence (e.g., 5′ splice site sequence) comprises GUC. In some embodiments, the splice site sequence (e.g., 5′ splice site sequence) comprises GUA. In some embodiments, the splice site sequence (e.g., 5′ splice site sequence) comprises GUG. In some embodiments, the splice site sequence (e.g., 5′ splice site sequence) comprises UCU. In some embodiments, the splice site sequence (e.g., 5′ splice site sequence) comprises UCC. In some embodiments, the splice site sequence (e.g., 5′ splice site sequence) comprises UCA. In some embodiments, the splice site sequence (e.g., 5′ splice site sequence) comprises UCG. In some embodiments, the splice site sequence (e.g., 5′ splice site sequence) comprises UUU. In some embodiments, the splice site sequence (e.g., 5′ splice site sequence) comprises UUC. In some embodiments, the splice site sequence (e.g., 5′ splice site sequence) comprises UUA. In some embodiments, the splice site sequence (e.g., 5′ splice site sequence) comprises UUG. In some embodiments, the splice site sequence (e.g., 5′ splice site sequence) comprises UGU. In some embodiments, the splice site sequence (e.g., 5′ splice site sequence) comprises UAU. In some embodiments, the splice site sequence (e.g., 5′ splice site sequence) comprises GGA. In some embodiments, the splice site sequence (e.g., 5′ splice site sequence) comprises CUU. In some embodiments, the splice site sequence (e.g., 5′ splice site sequence) comprises CUC. In some embodiments, the splice site sequence (e.g., 5′ splice site sequence) comprises CUA. In some embodiments, the splice site sequence (e.g., 5′ splice site sequence) comprises CUG. In some embodiments, the splice site sequence (e.g., 5′ splice site sequence) comprises CCU. In some embodiments, the splice site sequence (e.g., 5′ splice site sequence) comprises CCC. In some embodiments, the splice site sequence (e.g., 5′ splice site sequence) comprises CCA. In some embodiments, the splice site sequence (e.g., 5′ splice site sequence) comprises CCG. In some embodiments, the splice site sequence (e.g., 5′ splice site sequence) comprises ACU. In some embodiments, the splice site sequence (e.g., 5′ splice site sequence) comprises ACC. In some embodiments, the splice site sequence (e.g., 5′ splice site sequence) comprises ACG. In some embodiments, the splice site sequence (e.g., 5′ splice site sequence) comprises AGC. In some embodiments, the splice site sequence (e.g., 5′ splice site sequence) comprises AGU. In some embodiments, the splice site sequence (e.g., 5′ splice site sequence) comprises AGG. In some embodiments, the splice site sequence (e.g., 5′ splice site sequence) comprises CGU. In some embodiments, the splice site sequence (e.g., 5′ splice site sequence) comprises UAC. In some embodiments, the splice site sequence (e.g., 5′ splice site sequence) comprises UAA. In some embodiments, the splice site sequence (e.g., 5′ splice site sequence) comprises UAG. In some embodiments, the splice site sequence (e.g., 5′ splice site sequence) comprises CGC. In some embodiments, the splice site sequence (e.g., 5′ splice site sequence) comprises CGA. In some embodiments, the splice site sequence (e.g., 5′ splice site sequence) comprises CGG. In some embodiments, the splice site sequence comprises AGAguaaggg (SEQ ID NO: 667). In some embodiments, the splice site sequence comprises UGAguaagca (SEQ ID NO: 2768).

In an embodiment, a gene sequence or splice site sequence provided herein is related to a proliferative disease, disorder, or condition (e.g., cancer, benign neoplasm, or inflammatory disease). In an embodiment, a gene sequence or splice site sequence provided herein is related to a non-proliferative disease, disorder, or condition. In an embodiment, a gene sequence or splice site sequence provided herein is related to a neurological disease or disorder; autoimmune disease or disorder; immunodeficiency disease or disorder; lysosomal storage disease or disorder; cardiovascular condition, disease or disorder; metabolic disease or disorder; respiratory condition, disease, or disorder; renal disease or disorder; or infectious disease in a subject. In an embodiment, a gene sequence or splice site sequence provided herein is related to a neurological disease or disorder (e.g., Huntington's disease). In an embodiment, a gene sequence or splice site sequence provided herein is related to an immunodeficiency disease or disorder. In an embodiment, a gene sequence or splice site sequence provided herein is related to a lysosomal storage disease or disorder. In an embodiment, a gene sequence or splice site sequence provided herein is related to a cardiovascular condition, disease or disorder. In an embodiment, a gene sequence or splice site sequence provided herein is related to a metabolic disease or disorder. In an embodiment, a gene sequence or splice site sequence provided herein is related to a respiratory condition, disease, or disorder. In an embodiment, a gene sequence or splice site sequence provided herein is related to a renal disease or disorder. In an embodiment, a gene sequence or splice site sequence provided herein is related to an infectious disease.

In an embodiment, a gene sequence or splice site sequence provided herein is related to a mental retardation disorder. In an embodiment, a gene sequence or splice site sequence provided herein is related to a mutation in the SETD5 gene. In an embodiment, a gene sequence or splice site sequence provided herein is related to an immunodeficiency disorder. In an embodiment, a gene sequence and splice site sequence provided herein is related to a mutation in the GATA2 gene. In an embodiment, a gene sequence or splice site sequence provided herein is related to a lysosomal storage disease.

In some embodiments, a compound of Formula (I) or (II) described herein interacts with (e.g., binds to) a splicing complex component (e.g., a nucleic acid (e.g., an RNA) or a protein). In some embodiments, the splicing complex component is selected from 9G8, A1 hnRNP, A2 hnRNP, ASD-1, ASD-2b, ASF, BRR2, B1 hnRNP, C1 hnRNP, C2 hnRNP, CBP20, CBP80, CELF, F hnRNP, FBP11, Fox-1, Fox-2, G hnRNP, H hnRNP, hnRNP 1, hnRNP 3, hnRNP C, hnRNP G, hnRNP K, hnRNP M, hnRNP U, Hu, HUR, I hnRNP, K hnRNP, KH-type splicing regulatory protein (KSRP), L hnRNP, LUC7L, M hnRNP, mBBP, muscle-blind like (MBNL), NF45, NFAR, Nova-1, Nova-2, nPTB, P54/SFRS11, polypyrimidine tract binding protein (PTB), a PRP protein (e.g., PRP8, PRP6, PRP31, PRP4, PRP3, PRP28, PRP5, PRP2, PRP19), PRP19 complex proteins, RBM42, R hnRNP, RNPC1, SAD1, SAM68, SC35, SF, SF1/BBP, SF2, SF3A complex, SF3B complex, SFRS10, an Sm protein (such as B, D1, D2, D3, F, E, G), SNU17, SNU66, SNU114, an SR protein, SRm300, SRp20, SRp30c, SRP35C, SRP36, SRP38, SRp40, SRp55, SRp75, SRSF, STAR, GSG, SUP-12, TASR-1, TASR-2, TIA, TIAR, TRA2, TRA2a/b, U hnRNP, Ul snRNP, U11 snRNP, U12 snRNP, U1-70K, U1-A, U1-C, U2 snRNP, U2AF1-RS2, U2AF35, U2AF65, U4 snRNP, U5 snRNP, U6 snRNP, Urp, and YB1.

In some embodiments, the splicing complex component comprises RNA (e.g., snRNA). In some embodiments, a compound described herein binds to a splicing complex component comprising snRNA. The snRNA may be selected from, e.g., U1 snRNA, U2 snRNA, U4 snRNA, U5 snRNA, U6 snRNA, U11 snRNA, U12 snRNA, U4atac snRNA, and any combination thereof.

In some embodiments, the splicing complex component comprises a protein, e.g., a protein associated with an snRNA. In some embodiments, the protein comprises SC35, SRp55, SRp40, SRm300, SFRS10, TASR-1, TASR-2, SF2/ASF, 9G8, SRp75, SRp30c, SRp20 and P54/SFRS11. In some embodiments, the splicing complex component comprises a U2 snRNA auxiliary factor (e.g., U2AF65, U2AF35), Urp/U2AF1-RS2, SF1/BBP, CBP80, CBP 20, SF1 or PTB/hnRNP1. In some embodiments, the hnRNP protein comprises A1, A2/B1, L, M, K, U, F, H, G, R, I or C1/C2. Human genes encoding hnRNPs include HNRNPA0, HNRNPA1, HNRNPA1L1, HNRNP A1L2, HNRNPA3, HNRNPA2B1, HNRNP AB, HNRNPB1, HNRNPC, HNRNPCL1, HNRNPD, HNRPDL, HNRNPF, HNRNPH1, HNRNPH2, HNRNPH3, HNRNPK, HNRNPL, HNRPLL, HNRNPM, HNRNPR, HNRNPU, HNRNPUL1, HNRNPUL2, HNRNPUL3, and FMR1.

In one aspect, the compounds of Formula (I) or (II) and pharmaceutically acceptable salts, solvates, hydrates, tautomers, stereoisomers, and compositions thereof, may modulate (e.g., increase or decrease) a splicing event of a target nucleic acid sequence (e.g., DNA, RNA, or a pre-mRNA), for example, a nucleic acid encoding a gene described herein, or a nucleic acid encoding a protein described herein, or a nucleic acid comprising a splice site described herein. In an embodiment, the splicing event is an alternative splicing event.

In an embodiment, the compound of Formula (I) or (II) or a pharmaceutically acceptable salt, solvate, hydrate, tautomer, stereoisomer, and compositions thereof increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR. In an embodiment, the compound of Formula (I) or (II) or a pharmaceutically acceptable salt, solvate, hydrate, tautomer, stereoisomer, and compositions thereof decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR.

In another aspect, the present disclosure features a method of forming a complex comprising a component of a spliceosome (e.g., a major spliceosome component or a minor spliceosome component), a nucleic acid (e.g., a DNA, RNA, e.g., a pre-mRNA), and a compound of Formula (I) or (II) or a pharmaceutically acceptable salt, solvate, hydrate, tautomer, stereoisomer, or composition thereof, comprising contacting the nucleic acid (e.g., a DNA, RNA, e.g., a pre-mRNA) with said compound of Formula (I) or (II). In an embodiment, the component of a spliceosome is selected from the U1, U2, U4, U5, U6, U11, U12, U4atac, U6atac small nuclear ribonucleoproteins (snRNPs), or a related accessory factor. In an embodiment, the component of a spliceosome is recruited to the nucleic acid in the presence of the compound of Formula (I) or (II) or a pharmaceutically acceptable salt, solvate, hydrate, tautomer, stereoisomer, or composition thereof.

In another aspect, the present disclosure features a method of altering the conformation of a nucleic acid (e.g., a DNA, RNA, e.g., a pre-mRNA) comprising contacting the nucleic acid with a compound of Formula (I) or (II) or a pharmaceutically acceptable salt, solvate, hydrate, tautomer, stereoisomer, or composition thereof. In an embodiment, the altering comprises forming a bulge or kink in the nucleic acid. In an embodiment, the altering comprises stabilizing a bulge or a kink in the nucleic acid. In an embodiment, the altering comprises reducing a bulge or a kink in the nucleic acid. In an embodiment, the nucleic acid comprises a splice site. In an embodiment, the compound of Formula (I) or (II) interacts with a nucleobase, ribose, or phosphate moiety of a nucleic acid (e.g., a DNA, RNA, e.g., pre-mRNA).

The present disclosure also provides methods for the treatment or prevention of a disease, disorder, or condition. In an embodiment, the disease, disorder or condition is related to (e.g., caused by) a splicing event, such as an unwanted, aberrant, or alternative splicing event. In an embodiment, the disease, disorder or condition comprises a proliferative disease (e.g., cancer, benign neoplasm, or inflammatory disease) or non-proliferative disease. In an embodiment, the disease, disorder, or condition comprises a neurological disease, autoimmune disorder, immunodeficiency disorder, cardiovascular condition, metabolic disorder, lysosomal storage disease, respiratory condition, renal disease, or infectious disease in a subject. In another embodiment, the disease, disorder, or condition comprises a haploinsufficiency disease, an autosomal recessive disease (e.g., with residual function), or a paralogue activation disorder. In another embodiment, the disease, disorder, or condition comprises an autosomal dominant disorder (e.g., with residual function). Such methods comprise the step of administering to the subject in need thereof an effective amount of a compound of Formula (I) or (II), or a pharmaceutically acceptable salt, solvate, hydrate, tautomer, stereoisomer thereof, or a pharmaceutical composition thereof. In certain embodiments, the methods described herein include administering to a subject an effective amount of a compound of Formula (I) or (II), or a pharmaceutically acceptable salt thereof, or a pharmaceutical composition thereof.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

A proliferative disease may also be associated with inhibition of apoptosis of a cell in a biological sample or subject. All types of biological samples described herein or known in the art are contemplated as being within the scope of the disclosure. The compounds of Formula (I) or (II) and pharmaceutically acceptable salts, solvates, hydrates, tautomers, stereoisomers, and compositions thereof, may induce apoptosis, and therefore, be useful in treating and/or preventing proliferative diseases.

In certain embodiments, the proliferative disease to be treated or prevented using the compounds of Formula (I) or (II) is cancer. As used herein, the term “cancer” refers to a malignant neoplasm (Stedman's Medical Dictionary, 25th ed.; Hensyl ed.; Williams & Wilkins: Philadelphia, 1990). All types of cancers disclosed herein or known in the art are contemplated as being within the scope of the disclosure. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer), e.g., adenoid cystic carcinoma (ACC)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In some embodiments, the cancer is selected from adenoid cystic carcinoma (ACC), acute myelocytic leukemia (AML) (e.g., B-cell AML, T-cell AML), chronic myelocytic leukemia (CML) (e.g., B-cell CML, T-cell CML), non-Hodgkin lymphoma (NHL), Burkitt lymphoma, colorectal cancer (e.g., colon cancer, rectal cancer, colorectal adenocarcinoma), prostate cancer (e.g., prostate adenocarcinoma), ovarian cancer (e.g., cystadenocarcinoma, ovarian embryonal carcinoma, ovarian adenocarcinoma), and myelodysplastic syndrome (MDS).

In some embodiments, the proliferative disease is associated with a benign neoplasm. For example, a benign neoplasm may include adenoma, fibroma, hemangioma, tuberous sclerosis, and lipoma. All types of benign neoplasms disclosed herein or known in the art are contemplated as being within the scope of the disclosure.

In some embodiments, the proliferative disease is associated with angiogenesis. All types of angiogenesis disclosed herein or known in the art are contemplated as being within the scope of the disclosure.

In some embodiments, the compound of Formula (I) or (II) or a pharmaceutically acceptable salt thereof, or compositions comprising such compound or pharmaceutically acceptable salt thereof, is used to prevent or treat a non-proliferative disease. Exemplary non-proliferative diseases include a neurological disease, autoimmune disorder, immunodeficiency disorder, lysosomal storage disease, cardiovascular condition, metabolic disorder, respiratory condition, inflammatory disease, renal disease, or infectious disease.

In certain embodiments, the non-proliferative disease is a neurological disease. In certain embodiments, the compound of Formula (I) or (II), or a pharmaceutically acceptable salt thereof, or compositions comprising such compound or pharmaceutically acceptable salt thereof, is used to prevent or treat a neurological disease, disorder, or condition. A neurological disease, disorder, or condition may include a neurodegenerative disease, a psychiatric condition, or a musculoskeletal disease. A neurological disease may further include a repeat expansion disease, e.g., which may be characterized by the expansion of a nucleic acid sequence in the genome. For example, a repeat expansion disease includes myotonic dystrophy, amyotrophic lateral sclerosis, Huntington's disease, a trinucleotide repeat disease, or a polyglutamine disorder (e.g., ataxia, fragile X syndrome). In some embodiments, the neurological disease comprises a repeat expansion disease, e.g., Huntington's disease. Additional neurological diseases, disorders, and conditions include Alzheimer's disease, Huntington's chorea, a prion disease (e.g., Creutzfeld-Jacob disease, bovine spongiform encephalopathy, Kuru, or scrapie), a mental retardation disorder (e.g., a disorder caused by a SETD5 gene mutation, e.g., intellectual disability-facial dysmorphism syndrome, autism spectrum disorder), Lewy Body disease, diffuse Lewy body disease (DLBD), dementia, progressive supranuclear palsy (PSP), progressive bulbar palsy (PBP), psuedobulbar palsy, spinal and bulbar muscular atrophy (SBMA), primary lateral sclerosis, Pick's disease, primary progressive aphasia, corticobasal dementia, Parkinson's disease, Down's syndrome, multiple system atrophy, spinal muscular atrophy (SMA), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, a demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury), autism, Machado-Joseph disease, or a combination thereof. In some embodiments, the neurological disease comprises Friedrich's ataxia or Sturge Weber syndrome. In some embodiments, the neurological disease comprises Huntington's disease. In some embodiments, the neurological disease comprises spinal muscular atrophy. All types of neurological diseases disclosed herein or known in the art are contemplated as being within the scope of the disclosure.

In certain embodiments, the non-proliferative disease is an autoimmune disorder or an immunodeficiency disorder. In certain embodiments, the compound of Formula (I) or (II) or a pharmaceutically acceptable salt thereof, or compositions comprising such compound or pharmaceutically acceptable salt thereof, is used to prevent or treat an autoimmune disease, disorder, or condition, or an immunodeficiency disease, disorder, or condition. Exemplary autoimmune and immunodeficiency diseases, disorders, and conditions include arthritis (e.g., rheumatoid arthritis, osteoarthritis, gout), Chagas disease, chronic obstructive pulmonary disease (COPD), dermatomyositis, diabetes mellitus type 1, endometriosis, Goodpasture's syndrome, Graves' disease, Guillain-Barre syndrome (GBS), Hashiomoto's disease, Hidradenitis suppurativa, Kawasaki disease, ankylosing spondylitis, IgA nephropathy, idiopathic thrombocytopenia purpura, inflammatory bowel disease, Crohn's disease, ulcerative colitis, collagenous colitis, lymphocytic colitis, ischemic colitis, diversion colitis, Behcet's syndrome, infective colitis, indeterminate colitisinterstitial cystitis, lupus (e.g., systemic lupus erythematosus, discoid lupus, drug-induced lupus, neonatal lupus), mixed connective tissue disease, morphea, multiple sclerosis, myasthenia gravis, narcolepsy, neuromyotonia, pemphigus vulgaris, pernicious anemia, psoriasis, psoriatic arthritis, polymyositis, primary biliary cirrhosis, relapsing polychondritis, scleroderma, Sjögren's syndrome, Stiff person syndrome, vasculitis, vitiligo, a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), neutropenia, aplastic anemia, and Wegener's granulomatosis. In some embodiments, the autoimmune or immunodeficiency disorder comprises chronic mucocutaneous candidiasis. All types of autoimmune disorders and immunodeficiency disorders disclosed herein or known in the art are contemplated as being within the scope of the disclosure.

In certain embodiments, the non-proliferative disease is a cardiovascular condition. In certain embodiments, the compound of Formula (I) or (II) or a pharmaceutically acceptable salt thereof, or compositions comprising such compound or pharmaceutically acceptable salt thereof, is used to prevent or treat a cardiovascular disease, disorder, or condition. A cardiovascular disease, disorder, or condition may include a condition relating to the heart or vascular system, such as the arteries, veins, or blood. Exemplary cardiovascular diseases, disorders, or conditions include angina, arrhythmias (atrial or ventricular or both), heart failure, arteriosclerosis, atheroma, atherosclerosis, cardiac hypertrophy, cardiac or vascular aneurysm, cardiac myocyte dysfunction, carotid obstructive disease, endothelial damage after PTCA (percutaneous transluminal coronary angioplasty), hypertension including essential hypertension, pulmonary hypertension and secondary hypertension (renovascular hypertension, chronic glomerulonephritis), myocardial infarction, myocardial ischemia, peripheral obstructive arteriopathy of a limb, an organ, or a tissue; peripheral artery occlusive disease (PAOD), reperfusion injury following ischemia of the brain, heart or other organ or tissue, restenosis, stroke, thrombosis, transient ischemic attack (TIA), vascular occlusion, vasculitis, and vasoconstriction. All types of cardiovascular diseases, disorders, or conditions disclosed herein or known in the art are contemplated as being within the scope of the disclosure.

In certain embodiments, the non-proliferative disease is a metabolic disorder. In certain embodiments, the compound of Formula (I) or (II) or a pharmaceutically acceptable salt thereof, or compositions comprising such compound or pharmaceutically acceptable salt thereof, is used to prevent or treat a metabolic disease, disorder, or condition. A metabolic disease, disorder, or condition may include a disorder or condition that is characterized by abnormal metabolism, such as those disorders relating to the consumption of food and water, digestion, nutrient processing, and waste removal. A metabolic disease, disorder, or condition may include an acid-base imbalance, a mitochondrial disease, a wasting syndrome, a malabsorption disorder, an iron metabolism disorder, a calcium metabolism disorder, a DNA repair deficiency disorder, a glucose metabolism disorder, hyperlactatemia, a disorder of the gut microbiota. Exemplary metabolic conditions include obesity, diabetes (Type I or Type II), insulin resistance, glucose intolerance, lactose intolerance, eczema, hypertension, Hunter syndrome, Krabbe disease, sickle cell anemia, maple syrup urine disease, Pompe disease, and metachromatic leukodystrophy. All types of metabolic diseases, disorders, or conditions disclosed herein or known in the art are contemplated as being within the scope of the disclosure.

In certain embodiments, the non-proliferative disease is a respiratory condition. In certain embodiments, the compound of Formula (I) or (II) or a pharmaceutically acceptable salt thereof, or compositions comprising such compound or pharmaceutically acceptable salt thereof, is used to prevent or treat a respiratory disease, disorder, or condition. A respiratory disease, disorder, or condition can include a disorder or condition relating to any part of the respiratory system, such as the lungs, alveoli, trachea, bronchi, nasal passages, or nose. Exemplary respiratory diseases, disorders, or conditions include asthma, allergies, bronchitis, allergic rhinitis, chronic obstructive pulmonary disease (COPD), lung cancer, oxygen toxicity, emphysema, chronic bronchitis, and acute respiratory distress syndrome. All types of respiratory diseases, disorders, or conditions disclosed herein or known in the art are contemplated as being within the scope of the disclosure.

In certain embodiments, the non-proliferative disease is a renal disease. In certain embodiments, the compound of Formula (I) or (II) or a pharmaceutically acceptable salt thereof, or compositions comprising such compound or pharmaceutically acceptable salt thereof, is used to prevent or treat a renal disease, disorder, or condition. A renal disease, disorder, or condition can include a disease, disorder, or condition relating to any part of the waste production, storage, and removal system, including the kidneys, ureter, bladder, urethra, adrenal gland, and pelvis. Exemplary renal diseases include acute kidney failure, amyloidosis, Alport syndrome, adenovirus nephritis, acute lobar nephronia, tubular necrosis, glomerulonephritis, kidney stones, urinary tract infections, chronic kidney disease, polycystic kidney disease, and focal segmental glomerulosclerosis (FSGS). In some embodiments, the renal disease, disorder, or condition comprises HIV-associated nephropathy or hypertensive nephropathy. All types of renal diseases, disorders, or conditions disclosed herein or known in the art are contemplated as being within the scope of the disclosure.

In certain embodiments, the non-proliferative disease is an infectious disease. In certain embodiments, the compound of Formula (I) or (II) or a pharmaceutically acceptable salt thereof, or compositions comprising such compound or pharmaceutically acceptable salt thereof, is used to prevent or treat an infectious disease, disorder, or condition. An infectious disease may be caused by a pathogen such as a virus or bacteria. Exemplary infectious diseases include human immunodeficiency syndrome (HIV), acquired immunodeficiency syndrome (AIDS), meningitis, African sleeping sickness, actinomycosis, pneumonia, botulism, chlamydia, Chagas disease, Colorado tick fever, cholera, typhus, giardiasis, food poisoning, ebola hemorrhagic fever, diphtheria, Dengue fever, gonorrhea, streptococcal infection (e.g., Group A or Group B), hepatitis A, hepatitis B, hepatitis C, herpes simplex, hookworm infection, influenza, Epstein-Barr infection, Kawasaki disease, kuru, leprosy, leishmaniasis, measles, mumps, norovirus, meningococcal disease, malaria, Lyme disease, listeriosis, rabies, rhinovirus, rubella, tetanus, shingles, scarlet fever, scabies, Zika fever, yellow fever, tuberculosis, toxoplasmosis, or tularemia. In some embodiments, the infectious disease comprises cytomegalovirus. All types of infectious diseases, disorders, or conditions disclosed herein or known in the art are contemplated as being within the scope of the disclosure.

In certain embodiments, the disease, disorder, or condition is a haploinsufficiency disease. In certain embodiments, the compound of Formula (I) or (II) or a pharmaceutically acceptable salt thereof, or compositions comprising such compound or pharmaceutically acceptable salt thereof, is used to prevent or treat a haploinsufficiency disease, disorder, or condition. A haploinsufficiency disease, disorder, or condition may refer to a monogenic disease in which an allele of a gene has a loss-of-function lesion, e.g., a total loss of function lesion. In an embodiment, the loss-of-function lesion is present in an autosomal dominant inheritance pattern or is derived from a sporadic event. In an embodiment, the reduction of gene product function due to the altered allele drives the disease phenotype despite the remaining functional allele (i.e. said disease is haploinsufficient with regard to the gene in question). In an embodiment, a compound of Formula (I) or (II) increases expression of the haploinsufficient gene locus. In an embodiment, a compound of Formula (I) or (II) increases one or both alleles at the haploinsufficient gene locus. Exemplary haploinsufficiency diseases, disorders, and conditions include Robinow syndrome, cardiomyopathy, cerebellar ataxia, pheochromocytoma, Charcot-Marie-Tooth disease, neuropathy, Takenouchi-Kosaki syndrome, Coffin-Siris syndrome 2, chromosome 1p35 deletion syndrome, spinocerebellar ataxia 47, deafness, seizures, dystonia 9, GLUT1 deficiency syndrome 1, GLUT1 deficiency syndrome 2, stomatin-deficient cryohydrocytosis, basal cell carcinoma, basal cell nevus syndrome, medulloblastoma, somatic, brain malformations, macular degeneration, cone-rod dystrophy, Dejerine-Sottas disease, hypomyelinating neuropathy, Roussy-Levy syndrome, glaucoma, autoimmune lymphoproliferative syndrome, pituitary hormone deficiency, epileptic encephalopathy, early infantile, popliteal pterygium syndrome, van der Woude syndrome, Loeys-Dietz syndrome, Skraban-Deardorff syndrome, erythrocytosis, megalencephaly-polymicrogyria-polydactyly-hydrocephalus syndrome, mental retardation, CINCA syndrome, familial cold inflammatory syndrome 1, keratoendothelitis fugax hereditaria, Muckle-Wells syndrome, Feingold syndrome 1, Acute myeloid leukemia, Heyn-Sproul-Jackson syndrome, Tatton-Brown-Rahman syndrome, Shashi-Pena syndrome, Spastic paraplegia, autosomal dominant, macrophthalmia, colobomatous, with microcornea, holoprosencephaly, schizencephaly, endometrial cancer, familial, colorectal cancer, hereditary nonpolyposis, intellectual developmental disorder with dysmorphic facies and behavioral abnormalities, ovarian hyperstimulation syndrome, schizophrenia, Dias-Logan syndrome, premature ovarian failure, dystonia, dopa-responsive, due to sepiapterin reductase deficiency, Beck-Fahrner syndrome, chromosome 2p12-p11.2 deletion syndrome, neuronopathy, spastic paraplegia, familial adult myoclonic, colorectal cancer, hypothyroidism, Culler-Jones syndrome, holoprosencephaly, myelokathexis, WHIM syndrome, Mowat-Wilson syndrome, mental retardation, an intellectual developmental disorder, autism spectrum disorder, epilepsy, epileptic encephalopathy, Dravet syndrome, migraines, a mental retardation disorder (e.g., a disorder caused by a SETD5 gene mutation, e.g., intellectual disability-facial dysmorphism syndrome, autism spectrum disorder), a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), and febrile seizures.

In certain embodiments, the disease, disorder, or condition is an autosomal recessive disease, e.g., with residual function. In certain embodiments, the compound of Formula (I) or (II), or a pharmaceutically acceptable salt thereof, or compositions comprising such compound or pharmaceutically acceptable salt thereof, is used to prevent or treat an autosomal recessive disease, disorder, or condition. An autosomal recessive disease with residual function may refer to a monogenic disease with either homozygous recessive or compound heterozygous heritability. These diseases may also be characterized by insufficient gene product activity (e.g., a level of gene product greater than 0%). In an embodiment, a compound of Formula (I) or (II) may increase the expression of a target (e.g., a gene) related to an autosomal recessive disease with residual function. Exemplary autosomal recessive diseases with residual function include Friedreich's ataxia, Stargardt disease, Usher syndrome, chlorioderma, fragile X syndrome, achromatopsia 3, Hurler syndrome, hemophilia B, alpha-1-antitrypsin deficiency, Gaucher disease, X-linked retinoschisis, Wiskott-Aldrich syndrome, mucopolysaccharidosis (Sanfilippo B), DDC deficiency, epidermolysis bullosa dystrophica, Fabry disease, metachromatic leukodystrophy, and odontochondrodysplasia.

In certain embodiments, the disease, disorder, or condition is an autosomal dominant disease. In certain embodiments, the compound of Formula (I) or (II), or a pharmaceutically acceptable salt thereof, or compositions comprising such compound or pharmaceutically acceptable salt thereof, is used to prevent or treat an autosomal dominant disease, disorder, or condition. An autosomal dominant disease may refer to a monogenic disease in which the mutated gene is a dominant gene. These diseases may also be characterized by insufficient gene product activity (e.g., a level of gene product greater than 0%). In an embodiment, a compound of Formula (I) or (II) may increase the expression of a target (e.g., a gene) related to an autosomal dominant disease. Exemplary autosomal dominant diseases include Huntington's disease, achondroplasia, antithrombin III deficiency, Gilbert's disease, Ehlers-Danlos syndrome, hereditary hemorrhagic telangiectasia, intestinal polyposis, hereditary elliptosis, hereditary spherocytosis, marble bone disease, Marfan's syndrome, protein C deficiency, Treacher Collins syndrome, Von Willebrand's disease, tuberous sclerosis, osteogenesis imperfecta, polycystic kidney disease, neurofibromatosis, and idiopathic hypoparathyroidism.

In certain embodiments, the disease, disorder, or condition is a paralogue activation disorder. In certain embodiments, the compound of Formula (I) or (II), or a pharmaceutically acceptable salt thereof, or compositions comprising such compound or pharmaceutically acceptable salt thereof, is used to prevent or treat a paralogue activation disease, disorder, or condition. A paralogue activation disorder may comprise a homozygous mutation of genetic locus leading to loss-of-function for the gene product. In these disorders, there may exist a separate genetic locus encoding a protein with overlapping function (e.g. developmental paralogue), which is otherwise not expressed sufficiently to compensate for the mutated gene. In an embodiment, a compound of Formula (I) or (II) activates a gene connected with a paralogue activation disorder (e.g., a paralogue gene).

The cell described herein may be an abnormal cell. The cell may be in vitro or in vivo. In certain embodiments, the cell is a proliferative cell. In certain embodiments, the cell is a cancer cell. In certain embodiments, the cell is a non-proliferative cell. In certain embodiments, the cell is a blood cell. In certain embodiments, the cell is a lymphocyte. In certain embodiments, the cell is a benign neoplastic cell. In certain embodiments, the cell is an endothelial cell. In certain embodiments, the cell is an immune cell. In certain embodiments, the cell is a neuronal cell. In certain embodiments, the cell is a glial cell. In certain embodiments, the cell is a brain cell. In certain embodiments, the cell is a fibroblast. In certain embodiment, the cell is a primary cell, e.g., a cell isolated from a subject (e.g., a human subject).

In some embodiments, a compound of Formula (I) or (II) or a pharmaceutically acceptable salt thereof, e.g., as described herein, has improved cell permeability over a reference compound, e.g., in a standard assay for measuring cell permeability. Cell permeability may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I) or (II) or a pharmaceutically acceptable salt thereof, e.g., as described herein, has a cell permeability measurement (Papp) of <2×10⁻⁶ cm s⁻¹. In an embodiment, a compound of Formula (I) or (II) or a pharmaceutically acceptable salt thereof, e.g., as described herein, has a cell permeability measurement (Papp) of between 2-6×10⁻⁶ cm s⁻¹. In an embodiment, a compound of Formula (I) or (II) or a pharmaceutically acceptable salt thereof, e.g., as described herein, has a cell permeability measurement (Papp) of Papp greater than 6×10⁻⁶ cm s⁻¹. In an embodiment, a compound of Formula (I) or (II) or a pharmaceutically acceptable salt thereof, e.g., as described herein, has a cell permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or (II) or a pharmaceutically acceptable salt thereof, e.g., as described herein, exhibits decreased cell efflux, e.g., over a reference compound, e.g., in a standard assay for measuring cell efflux. Cell efflux may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I) or (II) or a pharmaceutically acceptable salt thereof, e.g., as described herein, has a cell efflux ratio of less than 1.5. In an embodiment, a compound of Formula (I) or (II) or a pharmaceutically acceptable salt thereof, e.g., as described herein, has a cell efflux ratio of between 1.5 and 5. In an embodiment, a compound of Formula (I) or (II) or a pharmaceutically acceptable salt thereof, e.g., as described herein, has a cell efflux ratio greater than 5. In an embodiment, a compound of Formula (I) or (II) or a pharmaceutically acceptable salt thereof, e.g., as described herein, has a cell efflux ratio less than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or (II) or a pharmaceutically acceptable salt thereof, e.g., as described herein, modulates the expression of a target protein (e.g., HTT or MYB) in a reference cell or sample. In an embodiment, a compound of Formula (I) or (II) or a pharmaceutically acceptable salt thereof, e.g., as described herein, increases the expression of a target protein (e.g., HTT or MYB) in a reference cell or sample. In an embodiment, a compound of Formula (I) or (II) or a pharmaceutically acceptable salt thereof, e.g., as described herein, decreases the expression of a target protein (e.g., HTT or MYB) in a reference cell or sample. The effect of an exemplary compound of Formula (I) or (II) on protein abundance may be measured using a standard assay for measuring protein abundance, such as the HiBit-assay system (Promega). In this assay, percent response for each respective cell line may be as calculated at each compound concentration as follows: % response=100*(S−PC)/(NC−PC). For the normalized response at each concentration, a four-parameter logistical regression may be fit to the data and the response may be interpolated at the 50% value to determine a concentration for protein abundance at 50% (IC₅₀) an untreated control. In an embodiment, a compound of Formula (I) or (II) or a pharmaceutically acceptable salt thereof, e.g., as described herein, has a protein abundance response less than 100 nM. In an embodiment, a compound of Formula (I) or (II) or a pharmaceutically acceptable salt thereof, e.g., as described herein, has a protein abundance response between 100-1000 nM. In an embodiment, a compound of Formula (I) or (II) or a pharmaceutically acceptable salt thereof, e.g., as described herein, has a protein abundance response greater than 1000 nM. In an embodiment, a compound of Formula (I) or (II) or a pharmaceutically acceptable salt thereof, e.g., as described herein, has a protein abundance response greater than 10 uM. In an embodiment, a compound of Formula (I) or (II) or a pharmaceutically acceptable salt thereof, e.g., as described herein, modulates the protein abundance of a target protein by about 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or (II) or a pharmaceutically acceptable salt thereof, e.g., as described herein, modulates the viability of a target cell in a subject or sample. In an embodiment, a compound of Formula (I) or (II) or a pharmaceutically acceptable salt thereof, e.g., as described herein, increases the viability of a target cell in a subject or sample. In an embodiment, a compound of Formula (I) or (II) or a pharmaceutically acceptable salt thereof, e.g., as described herein, decreases the viability of a target cell in a subject or sample. In an embodiment, a compound of Formula (I) or (II) or a pharmaceutically acceptable salt thereof, e.g., as described herein, does not impact the viability of a cell (e.g., is non-toxic) in a subject or sample. The effect an exemplary compound of Formula (I) or (II) on cell viability may be measured using a standard assay for measuring cell toxicity, such as the Cell Titer Glo 2.0 assay in either K562 (human chronic myelogenous leukemia) or SH-SY5Y (human neuroblastoma) cells. The concentration at which cell viability is measured may be based on the particular assay used. In an embodiment, a compound of Formula (I) or (II) or a pharmaceutically acceptable salt thereof, e.g., as described herein, is tolerated by a target cell at a concentration of less than 100 nM. In an embodiment, a compound of Formula (I) or (II) or a pharmaceutically acceptable salt thereof, e.g., as described herein, is tolerated by a target cell at a concentration of between 100-1000 nM. In an embodiment, a compound of Formula (I) or (II) or a pharmaceutically acceptable salt thereof, e.g., as described herein, is tolerated by a target cell at a concentration of greater than 1000 nM. In an embodiment, a compound of Formula (I) or (II) or a pharmaceutically acceptable salt thereof, e.g., as described herein, is tolerated by a target cell at a concentration of greater than 10 uM.

In some embodiments, a compound of Formula (I) or (II) or a pharmaceutically acceptable salt thereof, e.g., as described herein, has improved brain permeability over a reference compound, e.g., in a standard assay for measuring brain permeability. Brain permeability may be measured, for example, by determining the unbound partition coefficient (Kpuu), brain. In such an assay, the unbound brain partition coefficient (K_{p,uu,brain}) may be defined as the ratio of unbound brain-free compound concentration to unbound plasma concentration. It is calculated using the following equation:

$${K}_{p , uu , brain} = \frac{{f}_{u , brain} \times {C}_{brain}}{{f}_{u , plasma} \times {C}_{plasma}}$$

C_{brain} and Cₚₗₐₛₘₐ represent the total concentrations in brain and plasma, respectively. In this assay, the f_{u,brain} and f_{u,plasma} may be the unbound fraction of the compound in brain and plasma, respectively. Both f_{u,brain} and f_{u,plasma} may be determined in vitro via equilibrium dialysis. In an embodiment, a compound of Formula (I) or (II) or a pharmaceutically acceptable salt thereof, e.g., as described herein, has a Kp value of greater than 5. In an embodiment, a compound of Formula (I) or (II) or a pharmaceutically acceptable salt thereof, e.g., as described herein, has a Kp value between 1 and 5. In an embodiment, a compound of Formula (I) or (II) or a pharmaceutically acceptable salt thereof, e.g., as described herein, has a Kp value between 0.2-1. In an embodiment, a compound of Formula (I) or (II) or a pharmaceutically acceptable salt thereof, e.g., as described herein, has a Kp value of less than 0.2. In an embodiment, a compound of Formula (I) or (II) or a pharmaceutically acceptable salt thereof, e.g., as described herein, has a Kpuu value of greater than 2.5. In an embodiment, a compound of Formula (I) or (II) or a pharmaceutically acceptable salt thereof, e.g., as described herein, has a Kpuu value between 0.5-2.5. In an embodiment, a compound of Formula (I) or (II) or a pharmaceutically acceptable salt thereof, e.g., as described herein, has a Kpuu value between 0.1-0.5. In an embodiment, a compound of Formula (I) or (II) or a pharmaceutically acceptable salt thereof, e.g., as described herein, has a Kpuu value of less than 0.1. In an embodiment, a compound of Formula (I) or (II) or a pharmaceutically acceptable salt thereof, e.g., as described herein, has a brain permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or (II) or a pharmaceutically acceptable salt thereof, e.g., as described herein, exhibits selectivity for one target nucleic acid sequence, e.g., pre-mRNA transcript sequence or bulge, compared to another target nucleic acid sequence, e.g., pre-mRNA transcript sequence or bulge. In an embodiment, a compound of Formula (I) or (II) or a pharmaceutically acceptable salt thereof, e.g., as described herein, exhibits selectivity for HTT, e.g., an HTT-related nucleic acid sequence. In an embodiment, a compound of Formula (I) or (II) or a pharmaceutically acceptable salt thereof, e.g., as described herein, exhibits selectivity for SMN2, e.g., an SMN2-related nucleic acid sequence. In an embodiment, a compound of Formula (I) or (II) or a pharmaceutically acceptable salt thereof, e.g., as described herein, exhibits selectivity for Target C, e.g., a Target C-related nucleic acid sequence. In an embodiment, a compound of Formula (I) or (II) or a pharmaceutically acceptable salt thereof, e.g., as described herein, exhibits selectivity for MYB, e.g., a MYB-related nucleic acid sequence. Selectivity for one target nucleic acid sequence over another may be measured using any number of methods known in the art. In an embodiment, selectivity may be measured by determining the ratio of derived qPCR values (e.g., as described herein) for one target nucleic acid sequence over another. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for one target nucleic acid sequence over another. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over another target nucleic acid sequence. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over another. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over another target nucleic acid sequence. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for Target C sequence over another. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over MYB. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over HTT. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over SMN2. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over HTT. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over MYB. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over SMN2. In an embodiment, a compound of Formula (I) or (II) or a pharmaceutically acceptable salt thereof, e.g., as described herein, has a 3-fold greater selectivity for HTT over MYB. In an embodiment, a compound of Formula (I) or (II) or a pharmaceutically acceptable salt thereof, e.g., as described herein, has a 3-fold greater selectivity for MYB over HTT. In an embodiment, a compound of Formula (I) or (II) or a pharmaceutically acceptable salt thereof, e.g., as described herein, has a 10-fold greater selectivity for HTT over MYB. In an embodiment, a compound of Formula (I) or (II) or a pharmaceutically acceptable salt thereof, e.g., as described herein, has a 10-fold greater selectivity for MYB over HTT. In an embodiment, a compound of Formula (I) or (II) or a pharmaceutically acceptable salt thereof, e.g., as described herein, has a 3-fold greater selectivity for HTT over SMN2. In an embodiment, a compound of Formula (I) or (II) or a pharmaceutically acceptable salt thereof, e.g., as described herein, has a 3-fold greater selectivity for SMN2 over HTT. In an embodiment, a compound of Formula (I) or (II) or a pharmaceutically acceptable salt thereof, e.g., as described herein, has a 10-fold greater selectivity for HTT over SMN2. In an embodiment, a compound of Formula (I) or (II) or a pharmaceutically acceptable salt thereof, e.g., as described herein, has a 10-fold greater selectivity for SMN2 over HTT. In an embodiment, a compound of Formula (I) or (II) or a pharmaceutically acceptable salt thereof, e.g., as described herein, has a 3-fold greater selectivity for MYB over SMN2. In an embodiment, a compound of Formula (I) or (II) or a pharmaceutically acceptable salt thereof, e.g., as described herein, has a 3-fold greater selectivity for SMN2 over MYB. In an embodiment, a compound of Formula (I) or (II) or a pharmaceutically acceptable salt thereof, e.g., as described herein, has a 10-fold greater selectivity for MYB over SMN2. In an embodiment, a compound of Formula (I) or (II) or a pharmaceutically acceptable salt thereof, e.g., as described herein, has a 10-fold greater selectivity for SMN2 over MYB. In an embodiment, a compound of Formula (I) or (II) or a pharmaceutically acceptable salt thereof, e.g., as described herein, has a selectivity for one target nucleic acid sequence that is greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a second nucleic acid sequence.

In certain embodiments, the methods described herein comprise the additional step of administering one or more additional pharmaceutical agents in combination with the compound of Formula (I) or (II), a pharmaceutically acceptable salt thereof, or compositions comprising such compound or pharmaceutically acceptable salt thereof. Such additional pharmaceutical agents include, but are not limited to, anti-proliferative agents, anti-cancer agents, anti-diabetic agents, anti-inflammatory agents, immunosuppressant agents, and a pain-relieving agent. The additional pharmaceutical agent(s) may synergistically augment the modulation of splicing induced by the inventive compounds or compositions of this disclosure in the biological sample or subject. Thus, the combination of the inventive compounds or compositions and the additional pharmaceutical agent(s) may be useful in treating, for example, a cancer or other disease, disorder, or condition resistant to a treatment using the additional pharmaceutical agent(s) without the inventive compounds or compositions.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

Reactions can be purified or analyzed according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC). In some embodiments, absolute stereochemistry of chiral compounds provided herein is arbitrarily assigned.

### Proton NMR:

¹H NMR spectra were recorded in CDCl₃ solution in 5-mm o.d. tubes (Wildmad) at 24° C. and were collected on a BRUKER AVANCE NEO 400 at 400 MHz for ¹H. The chemical shifts (δ) are reported relative to tetramethylsilane (TMS=0.00 ppm) and expressed in ppm.

### Preparative HPLC Purification:

prep-HPLC purification was performed on a Waters-2545 or Shimadzu, using one of the following conditions:
Condition 1: Column: SunFire Prep C18 OBD Column 19*150 mm, 5 μm 10 nm; Mobile Phase A: water (0.05% HCl), Mobile Phase B: MeCN; Gradient 1: 30% B to 45% B in 7 min; Gradient 2: 10% B to 20% B in 7 min.
Condition 2: Column: SunFire Prep C18 OBD Column 19*150 mm, 5 μm 10 nm; Mobile Phase A: water (0.05% TFA), Mobile Phase B: MeCN; Gradient 1: 35% B to 55% B in 7 min; Gradient 2: 35% B to 50% B in 7 min; Gradient 3: 20% B to 50% B in 25 min; Gradient 4: 10% B to 20% B in 7 min; Gradient 5: 10% B to 30% B in 7 min; Gradient 6: 35% B to 60% B in 7 min; Gradient 7: 20% B to 30% B in 7 min; Gradient 8: 20% B to 50% B in 15 min; Gradient 9: 20% B to 40% B in 25 min; Gradient 10: 40% B to 60% B in 7 min; Gradient 11: 30% B to 70% B in 25 min; Gradient 12: 30% B to 40% B in 7 min; Gradient 13: 25% B to 35% B in 7 min; Gradient 14: 50% B to 60% B in 7 min.
Condition 3: Column: C18 silica gel; Mobile Phase A: water (0.1% NH₃·H₂O+10 mmol/L NH₄HCO₃), Mobile Phase B: MeCN; Gradient 1: 30% B to 80% B in 15 min.
Condition 4: Column: CHIRAL ART Cellulose-SZ, 3*25 cm, 5 μm; Mobile Phase A: CO₂, Mobile Phase B: (2:1) MeOH:DCM (0.1% 2M NH₃-MeOH); Gradient 1: 45% B isocratic.
Condition 5: Column: C18 silica gel, XBridge, 19×150 mm; Mobile Phase A: water (0.05% NH₃H₂O), Mobile Phase B: MeCN; Gradient 1: 30% B to 60% B in 7 min; Gradient 2: 25% B to 55% B in 7 min; Gradient 3: 35% B to 55% B in 7 min; Gradient 4: 40% B to 65% B in 7 min; Gradient 5: 27% B to 52% B in 8 min; Gradient 6: 50% B to 95% B in 8 min; Gradient 7: 45% B to 70% B in 14 min; Gradient 7a: 45% B to 70% B in 7 min; Gradient 8: 40% B to 60% B in 7 min; Gradient 9: 20% B to 60% B in 8 min; Gradient 10: 30% B to 70% B to 15 min; Gradient 10a: 30% B to 70% B in 8 min; Gradient 11: 40% B to 80% B in 7 min; Gradient 12: 50% B to 70% B in 10 min; Gradient 12a: 50% B to 70% B in 7 min; Gradient 13: 50% B to 60% B in 7 min; Gradient 14: 50% B to 75% B in 7 min; Gradient 15: 45% B to 85% B in 7 min; Gradient 16: 35% B to 85% B in 7 min; Gradient 17: 45% B to 65% B in 7 min; Gradient 18: 25% B to 45% B in 7 min; Gradient 19: 35% B to 75% B in 14 min; Gradient 19a: 35% B to 75% B in 7 min; Gradient 19b: 35% B to 75% B in 20 min Gradient 20: 35% B to 60% B in 14 min; Gradient 21: 40% B to 70% B in 7 min; Gradient 22: 15% B to 25% B in 7 min; Gradient 23: 45% B to 90% B in 7 min; Gradient 24: 40% B to 75% B in 12 min; Gradient 25: 50% B to 65% B in 7 min; Gradient 26: 5% B to 15% B in 30 min; Gradient 27: 55% B to 85% B in 14 min; Gradient 28: 65% B to 95% B in 7 min; Gradient 29: 50% B to 80% B in 7 min; Gradient 30: 50% B to 90% B in 7 min; Gradient 31: 50% B to 85% B in 7 min; Gradient 32: 65% B to 85% B in 7 min; Gradient 33: 55% B to 95% B in 7 min; Gradient 34: 60% B to 80% B in 7 min.
Condition 6: Column: SunFire Prep 19*150 mm, 10 nm; Mobile Phase A: water (0.05% NH₄OH), Mobile Phase B: MeCN; Gradient 1: 35% B to 50% B in 7 min; Gradient 2: 10% B to 25% B in 7 min; Gradient 3: 50% B to 60% B in 7 min; Gradient 4: 40% B to 50% B in 7 min; Gradient 5: 40% B to 70% B in 10 min; Gradient 6: 35% B to 45% B in 7 min; Gradient 7: 40% B to 60% B in 7 min; Gradient 8: 20% B to 30% B in 7 min; Gradient 9: 50% B to 70% B in 10 min; Gradient 10: 30% B to 40% B in 7 min; Gradient 11: 20% B to 35% B in 7 min; Gradient 12: 30% B to 60% B in 7 min.
Condition 7: Column: XBridge BEH Shield RP18 5 m, 30 mm*150 mm; Mobile Phase A: Water (10 mmol/L NH₄HCO₃), Mobile Phase B: MeCN; Flow rate: 60 mL/min; Wave Length: UV 254 nm/220 nm; Gradient 1: 15% B to 35% B in 8 min.
Condition 8: Column: Kinetex 5 m EVO C18, 30 mm*150 mm; Mobile Phase A: Water (10 mmol/L NH₄HCO₃); Mobile Phase B: MeCN; Flow rate: 60 mL/min; Wave Length: UV 254 nm/220 nm; Gradient 1: 20% B to 46% B in 8 min; Gradient 2: 28% B to 46% B in 10 min; Gradient 3: 10% B to 40% B in 8 min.
Condition 9: Column: Phenomenex luna C18 80*30 mm*3 um; Mobile Phase A: H₂O (0.04% HCl); Mobile Phase B: MeCN; Gradient 1: 25% B to 65% B in 8 min; Gradient 2: 5% B to 35% B in 8 min; Gradient 3: 5% B to 45% B in 8 min; Gradient 4: 15% B to 40% B in 8 min; Gradient 5: 10% B to 35% B in 8 min; Gradient 6: 30% B to 60% B in 8 min.
Condition 10: Column: Phenomenex luna C18 100*40 mm*5 um; Mobile Phase A: H₂O (0.04% HCl); Mobile Phase B: MeCN; Gradient 1: 25% B to 55% B in 8 min.
Condition 11: Column: Phenomenex Luna C18 100*30 mm*5 um; Mobile Phase A: H₂O (0.2% HCl); Mobile Phase B: MeCN; Gradient 1: 1% B to 30% B in 8 min.
Condition 12: Column: Waters Xbridge C18 150*50 mm*10 um; Mobile Phase A: H₂O (10 mM NH₄HCO₃); Mobile Phase B: MeCN; Gradient 1: 50% B to 80% B in 8 min.
Condition 13: Column: XBride Prep OBD Column, 19*150 mm, 8 um; Mobile Phase A: water (0.1% NH₃·H₂O); Mobile Phase B: MeCN; Flow rate: 10 mL/min; Wave Length: 254 nm; Gradient 1: 10% B to 50% B in 8 min.
Condition 14: Column: XBride Prep OBD Column, 19*150 mm, 8 um; Mobile Phase A: water (0.05% NH₃H₂O); Mobile Phase B: MeCN; Flow rate: 20 mL/min; Wave Length: 220 nm; Gradient 1: 27% B to 52% B in 8 min; Gradient 2: 35% B to 55% B in 7 min; Gradient 3: 50% B to 75% B in 7 min; Gradient 4: 45% B to 60% B in 7 min; Gradient 5: 45% B to 65% B in 7 min; Gradient 6: 55% B to 90% B in 7 min.
Condition 15: Column: Weich Ultimate XB—C18 50×250 mm 10 um; Mobile Phase A: water (0.1% TFA); Mobile Phase B: MeCN; Flow rate: 90 mL/min; Gradient 1: 20% B to 60% B in 12 min; Gradient 2: 35% B to 65% B in 12 min, 65% isocratic 3 min.
Condition 16: Column: C18 silica gel, XBridge, 19×150 mm; Mobile Phase A: water (0.05% TFA); Mobile Phase B: MeCN; Gradient 1: 30% B to 50% B in 10 min; Gradient 2: 15% B to 45% B in 8 min.
Condition 17: Column: C18 silica gel, XBridge, 19×150 mm; Mobile Phase A: water (0.05% FA); Mobile Phase B: MeCN; Gradient 1: 5% B to 45% B in 12 min; Gradient 2: 10% B to 15% B in 10 min.
Condition 18: Column: Xbridge Prep C18 5 um, OBD, 30×150 mm; Mobile Phase A: water (0.1% NH₃·H₂O+10 mmol NH₄HCO₃); Mobile Phase B: MeCN; Gradient 1: 10% B to 58% B in 8.5 min; Gradient 2: 7% to 73% B in 7 min.

### Reversed-Phase Flash Chromatography:

Reversed-Phase Flash Chromatography was performed using one of the following conditions:
Condition 1: Column: C18 silica gel; Mobile Phase A, water (0.1% NH₃·H₂O+10 mmol/L NH₄HCO₃), Mobile Phase B: MeCN; Gradient 1: 30% B to 80% B in 12 min; Gradient 2: 15% B to 60% B in 12 min; Gradient 3: 15% B to 50% B in 12 min; Gradient 4: 30% B to 70% B in 10 min; Gradient 5: 20% B to 70% B in 10 min; Gradient 6: 10% B to 50% B in 10 min; Gradient 7: 40% B to 90% B in 12 min.
Condition 2: Column: C18 silica gel; Mobile Phase A: water (0.1% NH₃·H₂O), Mobile Phase B: MeOH; Gradient 1: 60% B to 85% B in 10 min.
Condition 3: Column: C18 silica gel; Mobile Phase A: water (0.1% NH₃·H₂O), Mobile Phase B: MeCN; Gradient 1: 40% B to 90% B in 12 min; Gradient 2: 5% B to 45% B in 12 min; Gradient 3: 10% B to 50% B in 10 min; Gradient 4: 30% B to 60% B in 10 min; Gradient 5: 30% B to 80% B in 12 min; Gradient 5a: 30% B to 80% B in 10 min; Gradient 6: 30% B to 80% B in 10 min; Gradient 7: 20% B to 60% B in 10 min; Gradient 8: 40% B to 70% B in 10 min; Gradient 9: 20% B to 50% B in 10 min; Gradient 10: 60% B to 80% B in 10 min; Gradient 11: 25% B to 50% B in 10 min; Gradient 12: 45% B to 86% B in 10 min; Gradient 13: 10% B to 40% B in 10 min; Gradient 14: 75% B to 80% B in 10 min; Gradient 15: 45% B to 70% B in 10 min; Gradient 16: 25% B to 80% B in 10 min; Gradient 17: 20% B to 65% B in 12 min; Gradient 18: 50% B to 80% B in 10 min; Gradient 19: 40% B to 60% B in 10 min; Gradient 20: 50% B to 90% B in 10 min; Gradient 21: 60% B to 75% B in 10 min; Gradient 22: 50% B to 70% B in 10 min; Gradient 23: 65% B to 80% B in 10 min; Gradient 24: 55% B to 75% B in 10 min; Gradient 25: 45% B to 75% B in 10 min; Gradient 26: 60% B to 90% B in 10 min; Gradient 27: 30% B to 70% B in 10 min; Gradient 28: 5% to 35% B in 12 min; Gradient 29: 45% B to 85% B in 15 min; Gradient 30: 15% B to 60% B in 12 min; Gradient 31: 40% B to 80% B in 10 min; Gradient 32: 20% B to 55% B in 12 min; Gradient 33: 60% B to 85% B in 10 min; Gradient 34: 45% B to 85% B in 10 min; Gradient 35: 20% B to 60% B in 12 min; Gradient 36: 25% B to 85% B in 10 min; Gradient 37: 35% B to 80% B in 10 min; Gradient 38: 25% B to 70% B in 10 min; Gradient 39: 45% B to 90% B in 10 min; Gradient 40: 45% B to 80% B in 10 min; Gradient 41: 50% B to 85% B in 10 min; Gradient 42: 45% B to 82% B in 10 min; Gradient 43: 5% B to 30% B in 10 min.
Condition 4: Column: C18 silica gel; Mobile Phase A: water (0.1% TFA), Mobile Phase B: MeCN; Gradient 1: 10% B to 50% B in 10 min; Gradient 2: 25% B to 50% B in 10 min; Gradient 3: 5% B to 30% B in 10 min; Gradient 4: 10% B to 60% B in 10 min; Gradient 5: 20% B to 60% B in 10 min; Gradient 6: 20% B to 65% B in 20 min; Gradient 7: 15% B to 50% B in 10 min.
Condition 5: Column: C18 silica gel; Mobile Phase A: water (0.05% NH₃H₂O), Mobile Phase B: MeCN; Gradient 1: 10% B to 50% B in 10 min.
Condition 6: Column: SunFire Prep C18 OBD 19*150 mm, 5 μm 10 nm; Mobile Phase A: water (0.05% TFA), Mobile Phase B: MeCN; Gradient 1: 10% B to 20% B in 7 min; Gradient 2: 10% B to 65% B in 15 min; Gradient 3: 10% B to 30% B in 7 min.
Condition 7: Column: C18 silica gel; Mobile Phase A: water (0.05% TFA), Mobile Phase B: MeCN; Gradient 1: 30% B to 60% B in 7 min.
Condition 8: Column: C18 silica gel; Mobile Phase A: water (0.1% FA), Mobile Phase B: MeCN; Gradient 1: 5% B to 30% B in 12 min.
Condition 9: Column: SunFire Prep C18 OBD 19*150 mm, 5 μm 10 nm; Mobile Phase B: MeCN in water (0.1% TFA); Gradient 1: 25% B to 70% B in 20 min.
Condition 10: Column: C18 silica gel; Mobile Phase A: water (0.1% TFA), mobile B: MeCN; Gradient 1: 25% B to 50% B in 10 min; Gradient 2: 10% b to 50% B in 10 min.

### Preparative Chiral HPLC:

purification by chiral HPLC was performed purification by chiral HPLC was performed on a Gilson-GX 281 using column CHIRAL ART, or CHIRALPAK IG using one of the following conditions:
Condition 1: Column: CHIRAL ART Cellulose-SC 3*25 cm, 5 μm; Mobile Phase A: EtOH (0.1% 2M NH₃-MeOH), Mobile Phase B: DCM (0.1% 2M NH₃-MeOH); Gradient 1: isocratic 50% B.
Condition 2: Column: CHIRALPAK IG, 3*25 cm, 5 μm; Mobile Phase A: HEX:DCM=3:1, Mobile Phase B: EtOH (0.1% IPAmine); Gradient 1: isocratic 50% B.
Condition 3: Column: YMC Cellulose-SZ 50*4.6 mm, 3 um; Mobile Phase A: n-Hexane/DCM=3/1; Mobile Phase B: EtOH (0.1% MIPA); Total Flow: 1 mL/min; Gradient 1: 50% B.
Condition 4: Column: YMC Cellulose-SC, 100*4.6 mm, 3 um; Mobile Phase A: DCM; Mobile Phase B: EtOH (20 mM NH₃); Gradient 1: isocratic.
Condition 5: CHIRAL ART Cellulose-SB 3*25 cm, 5 μm; Mobile Phase A: CO₂; Mobile Phase B: MeOH:DCM=2:1 (0.1% 2M NH₃-MeOH); Flow rate: 85 mL/min; Column Temperature (° C.): 35; Back Pressure (bar): 100; Gradient 1: isocratic 45% B.

### Example 1: Synthesis of Compound 208

### Synthesis of Intermediate A2

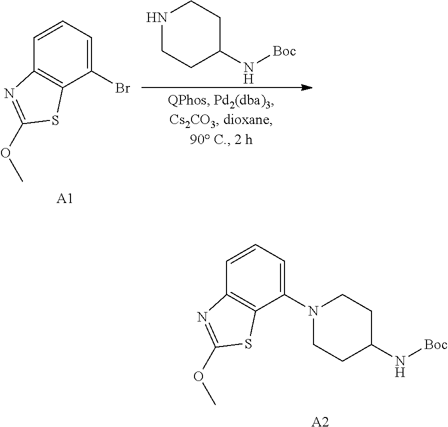

To a stirred mixture of 7-bromo-2-methoxy-1,3-benzothiazole (A1, 1 g, 4.12 mmol, 1 eq) and tert-butyl piperidin-4-ylcarbamate (0.99 g, 4.94 mmol, 1.2 eq) in dioxane (20 mL) were added QPhos (0.39 g, 0.82 mmol, 0.2 eq), Pd₂(dba)₃ (0.39 g, 0.41 mmol, 0.1 eq) and Cs₂CO₃ (2.87 g, 8.23 mmol, 2 eq) in portions, and the reaction was stirred for 2 h at 90° C. under N₂. The resulting mixture was concentrated under reduced pressure and purified by silica gel column chromatography (50% EtOAc in PE) to afford tert-butyl (1-(2-methoxybenzo[d]thiazol-7-yl)piperidin-4-yl)carbamate (A2, 0.8 g). LCMS (ES, m/z): 364 [M+H]⁺.

### Synthesis of Intermediate A3

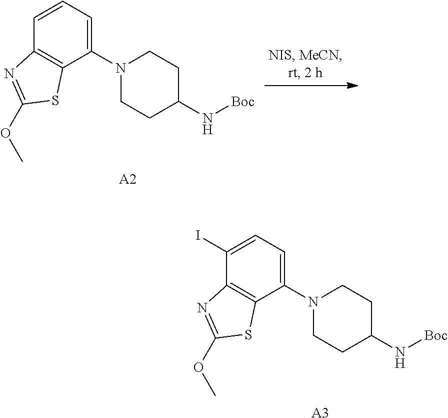 [SMILES: COc1nc2cccc(N3CCC(NC(=O)OC(C)(C)C)CC3)c2s1 ; "A2" ; COc1nc2c(I)ccc(N3CCC(NC(=O)OC(C)(C)C)CC3)c2s1 ; "A3"]

To a solution of Intermediate A2 (800 mg, 2.2 mmol, 1 eq) in MeCN (8 mL) was added NIS (761 mg, 4.4 mmol, 2 eq) in portions at room temperature. The resulting mixture was stirred for 2 h, quenched with water (20 mL), and then extracted with EtOAc (3×20 mL). The combined organics layer was washed brine (2×5 mL), dried over anhydrous Na₂SO₄, and filtered. The filtrate was concentrated under reduced pressure and purified by silica gel column chromatography (50% EtOAc in PE) to afford tert-butyl (1-(4-iodo-2-methoxybenzo[d]thiazol-7-yl)piperidin-4-yl)carbamate (A3, 650 mg). LCMS (ES, m/z): 490 [M+H]⁺.

### Synthesis of Intermediate A4

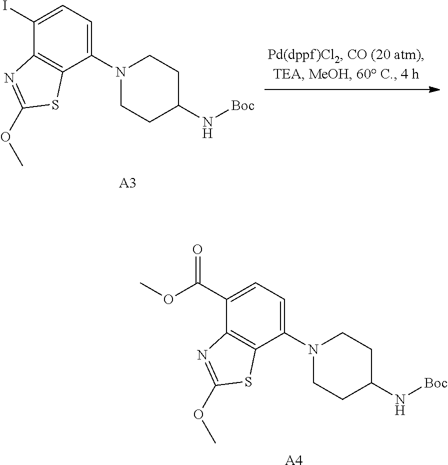 [SMILES: COc1nc2c(I)ccc(N3CCC(NC(=O)OC(C)(C)C)CC3)c2s1 ; "A3" ; COC(=O)c1ccc(N2CCC(NC(=O)OC(C)(C)C)CC2)c2sc(OC)nc12 ; "A4"]

To a solution of Intermediate A3 (650 mg, 1.33 mmol, 1 eq) in MeOH (15 mL) was added Pd(dppf)Cl₂ (98 mg, 0.13 mmol, 0.1 eq) and TEA (959 mg, 3.98 mmol, 3 eq) in a pressure tank. The mixture was purged with N₂ for 1 min and then was pressurized to 20 atm with carbon monoxide at 60° C. for 4 h. The resulting mixture was cooled to room temperature and filtered to remove insoluble solids. The filtrate was concentrated under reduced pressure and purified by silica gel column chromatography (50% EtOAc in PE) to afford methyl 7-(4-((tert-butoxycarbonyl)amino)piperidin-1-yl)-2-methoxybenzo[d]thiazole-4-carboxylate (A4, 500 mg). LCMS (ES, m/z): 422 [M+H]⁺.

### Synthesis of Intermediate A5

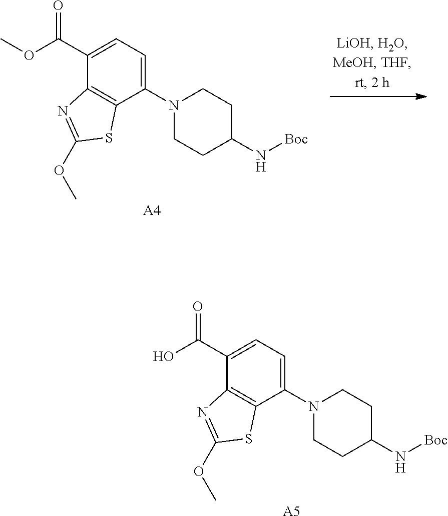 [SMILES: COC(=O)c1ccc(N2CCC(NC(=O)OC(C)(C)C)CC2)c2sc(OC)nc12 ; "A4" ; COc1nc2c(C(=O)O)ccc(N3CCC(NC(=O)OC(C)(C)C)CC3)c2s1 ; "A5"]

A mixture of Intermediate A4 (500 mg, 1.2 mmol, 1 eq) and LiOH (142 mg, 5.92 mmol, 5 eq) in THF (5 mL), MeOH (5 mL) and H₂O (2 mL) was stirred for 2 h at room temperature and then acidified to pH 5 with HCl (aq.). The resulting solids were collected by filtration and washed with water (3×10 mL) to afford 7-(4-((tert-butoxycarbonyl)amino)piperidin-1-yl)-2-methoxybenzo[d]thiazole-4-carboxylic acid (A5, 400 mg). LCMS (ES, m/z): 408 [M+H]⁺.

### Synthesis of Intermediate A6

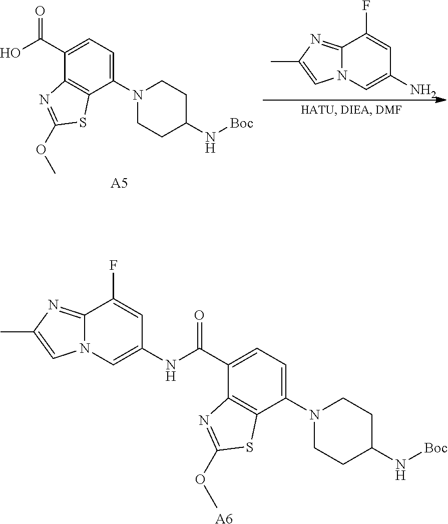

To a solution of Intermediate A5 (400 mg, 0.98 mmol, 1 eq) and DIEA (270 mg, 2.94 mmol, 3 eq) in DMF (5 mL) were added HATU (558 mg, 1.47 mmol, 1.5 eq) and 8-fluoro-2-methylimidazo[1,2-a]pyridin-6-amine dihydrochloride (695 mg, 2.94 mmol, 1.5 eq) at room temperature. The resulting mixture was stirred for 2 h, then quenched with water (20 mL), and the precipitated solids were collected by filtration and washed with water (3×5 mL) to afford tert-butyl (1-(4-((8-fluoro-2-methylimidazo[1,2-a]pyridin-6-yl)carbamoyl)-2-methoxybenzo[d]-thiazol-7-yl)piperidin-4-yl)carbamate (A6, 220 mg). LCMS (ES, m/z): 555 [M+H]⁺.

### Synthesis of Compound 208

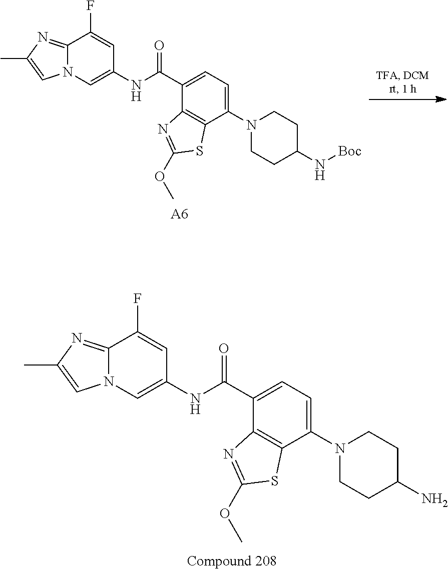

A mixture of Intermediate A6 (220 mg, 0.4 mmol, 1 eq) and TFA (0.5 mL) in DCM (1 mL) was stirred for 1 h at room temperature. The resulting mixture was concentrated under reduced pressure and purified by reversed-phase flash chromatography (Condition 1, Gradient 1) to afford 7-(4-aminopiperidin-1-yl)-N-(8-fluoro-2-methylimidazo[1,2-a]pyridin-6-yl)-2-methoxybenzo[d]thiazole-4-carboxamide (Compound 208, 120 mg). LCMS (ES, m/z): 455 [M+H]⁺.

An analogous method was followed to obtain the following compound.

| Compound | Starting Material | Characterization |
|---|---|---|
| 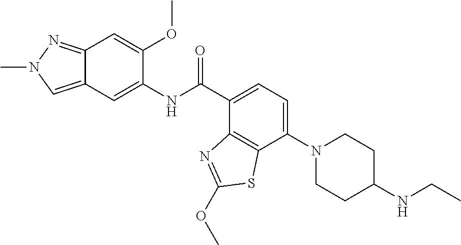 [SMILES: CCNC1CCN(c2ccc(C(=O)Nc3cc4cn(C)nc4cc3OC)c3nc(OC)sc23)CC1] Compound 669 | 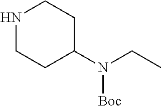 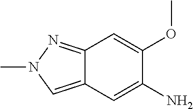 | Modifications: Step 1 was ran using Pd-PEPPSI-IPentCl for 16 h. Step 2 was ran in DCM at 0° C. to room temperature. Step 3 was ran for 2 h. Step 5 was ran using TCFH and NMI in MeCN. Step 6 was ran for 2 h. The crude product was purified by Prep-HPLC (Condition 5, Gradient 27) to afford 7-[4-(ethylamino)-1- piperidyl]-2-methoxy-N-(6- methoxy-2-methyl-indazol-5-yl)- 1,3-benzo-thiazole-4-carboxamide (Compound 669, 14 mg, 28.3 μmol). LCMS (ES, m/z): 495 [M + H]⁺. ¹H NMR (400 MHz, DMSO-d₆) δ 11.43 (s, 1H), 8.81 (s, |
| | | 1H), 8.38-8.09 (m, 2H), 7.23-6.94 |
| | | (m, 2H), 4.41 (s, 3H), 4.10 (s, 3H), |
| | | 3.95 (s, 3H), 3.55 (d, J = 12.4 Hz, |
| | | 2H), 2.91 (t, J = 11.4 Hz, 2H), 2.60 |
| | | (q, J = 7.0 Hz, 3H), 1.97 (d, J = 12.5 |
| | | Hz, 2H), 1.42 (q, J = 10.9, 10.3 Hz, |
| | | 2H), 1.04 (t, J = 7.1 Hz, 3H). |
| 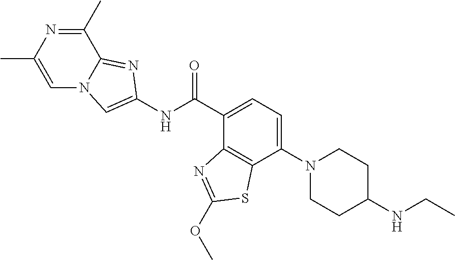 [SMILES: CCNC1CCN(c2ccc(C(=O)Nc3cn4cc(C)nc(C)c4n3)c3nc(OC)sc23)CC1] Compound 670 | 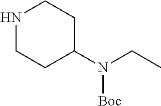 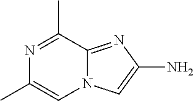 | Modifications: Step 1 was ran using Pd-PEPPSI-IPentCl for 16 h. Step 2 was ran in DCM at 0° C. to room temperature. Step 3 was ran for 2 h. Step 5 was ran using TCFH and NMI in MeCN. Step 6 was ran for 2 h. The crude product was purified by Prep-HPLC (Condition 5, Gradient 28) to afford N-(6,8-dimethyl- imidazo[1,2-a]-pyrazin-2-yl)-7-[4- (ethylamino)-1-piperidyl]-2- methoxy-1,3-benzothiazole-4- carboxamide (Compound 670, 19 mg, 39.6 μmol). LCMS (ES, m/z): 480 [M + H]⁺. ¹H NMR (400 MHz, DMSO-d₆) δ 12.01 (s, 1H), 8.32 (d, J = 1.9 Hz, 2H), 8.17 (d, J = 8.5 Hz, |
| | | 1H), 7.09 (d, J = 8.6 Hz, 1H), 4.40 |
| | | (s, 3H), 3.58 (d, J = 12.3 Hz, 2H), |
| | | 2.94 (t, J = 11.5 Hz, 2H), 2.67 (s, |
| | | 3H), 2.61 (q, J = 7.1 Hz, 3H), 2.38 |
| | | (s, 3H), 1.97 (d, J = 12.6 Hz, 2H), |
| | | 1.43 (q, J = 10.3 Hz, 2H), 1.04 (t, J = |
| | | 7.1 Hz, 3H). |
| 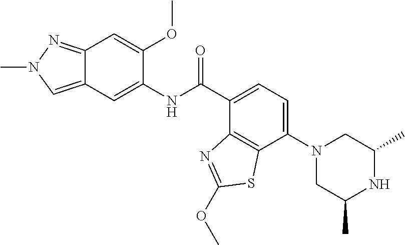 Compound 671 | 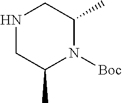 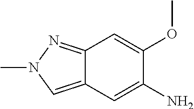 | Modifications: Step 1 was ran using QPhos Pd G3 for 5 h. Step 2 was ran in DCM at 0° C. to room temperature. Step 3 was ran for 5 h. Step 5 was ran using TCFH and NMI in MeCN at room temperature for 2 h. Step 6 was ran for 2 h. The residue was purified by reversed-phase flash chromatography (Condition 3, Gradient 41) to afford 7-[(3S,5S)- 3,5-dimethyl-piperazin-1-yl]-2- methoxy-N-(6-methoxy-2-methyl- indazol-5-yl)-1,3-benzothiazole-4- carboxamide (Compound 671, 18 mg, 37.5 μmol). LCMS (ES, m/z): 481 [M + H]⁺. 1H NMR (400 MHz, |
| | | DMSO-d₆) δ 11.43 (s, 1H), 8.81 (s, |
| | | 1H), 8.20 (d, J = 9.1 Hz, 2H), 7.11 |
| | | (s, 1H), 7.06 (d, J = 8.6 Hz, 1H), |
| | | 4.40 (s, 3H), 4.10 (s, 3H), 3.94 (s, |
| | | 3H), 3.23 (td, J = 6.3, 3.2 Hz, 2H), |
| | | 3.10 (dd, J = 11.4, 3.1 Hz, 2H), |
| | | 2.91 (dd, J = 11.4, 6.1 Hz, 2H), |
| | | 2.17-2.15 (m, 1H), 1.17 (d, J = 6.5 |
| | | Hz, 6H). |

### Example 2: Synthesis of Compound 100

### Synthesis of Intermediate A8

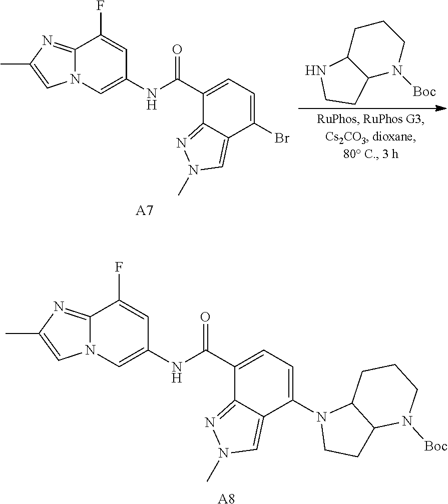

To a stirred mixture of 4-bromo-N-(8-fluoro-2-methylimidazo[1,2-a]pyridin-6-yl)-2-methylindazole-7-carboxamide (A7, 300 mg, 0.75 mmol, 1 eq) and tert-butyl octahydropyrrolo[3,2-b]pyridine-4-carboxylate (202 mg, 0.9 mmol, 1.2 eq) in dioxane (5 mL) were added RuPhos (70 mg, 0.15 mmol, 0.2 eq), Cs₂CO₃ (730 mg, 2.24 mmol, 3 eq) and RuPhos Pd G3 (62 mg, 0.08 mmol, 0.1 eq), and the reaction was stirred for 3 h at 80° C. under N₂. The resulting mixture was concentrated under reduced pressure and purified by silica gel column chromatography (90% EtOAc in PE) to afford tert-butyl 1-[7-((8-fluoro-2-methylimidazo[1,2-a]-pyridin-6-yl)carbamoyl)-2-methylindazol-4-yl]-hexahydro-2H-pyrrolo[3,2-b]pyridine-4-carboxylate (A8, 230 mg). LCMS (ES, m/z): 548 [M+H]⁺.

### Synthesis of Compound 100

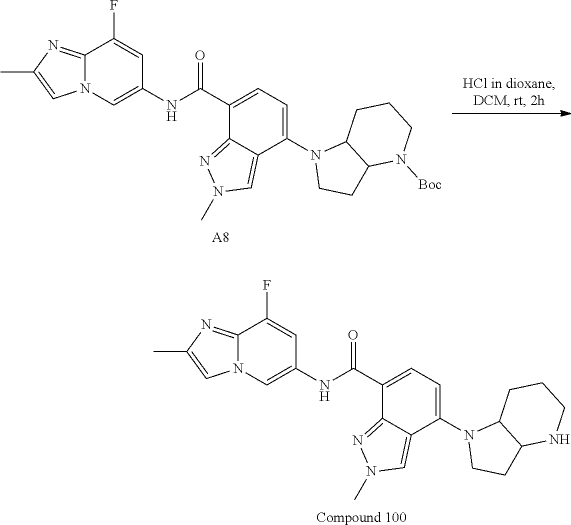

To a stirred solution of Intermediate A8 (230 mg, 0.42 mmol, 1 eq) in DCM (3 mL) was added HCl (gas) in 1,4-dioxane (1 mL, 4 M) and the reaction was stirred for 2 h at room temperature. The resulting mixture was concentrated under reduced pressure and purified by reversed-phase flash chromatography (Condition 2, Gradient 1) to afford N-(8-fluoro-2-methylimidazo[1,2-a]pyridin-6-yl)-2-methyl-4-(octahydropyrrolo[3,2-b]pyridin-1-yl)indazole-7-carboxamide (Compound 100, 120 mg). LCMS (ES, m/z): 448 [M+H]⁺. ¹H NMR (300 MHz, DMSO-d₆) δ 11.02 (s, 1H), 9.19 (d, J=1.6 Hz, 1H), 8.75 (s, 1H), 7.95-7.84 (m, 2H), 7.30 (dd, J=12.4, 1.7 Hz, 1H), 6.11 (d, J=8.4 Hz, 1H), 4.27 (s, 3H), 3.91-3.89 (m, 2H), 3.66 (q, J=8.3 Hz, 1H), 3.62-3.51 (m, 1H), 2.72-2.65 (m, 2H), 2.65 (d, J=12.9 Hz, 3H), 2.31-2.19 (m, 1H), 2.06-2.05 (m, 1H), 1.85-1.84 (m, 1H), 1.56-1.45 (m, 3H).

An analogous method was followed to obtain the following compounds.

| Compound | Starting Material | Characterization |
|---|---|---|
| 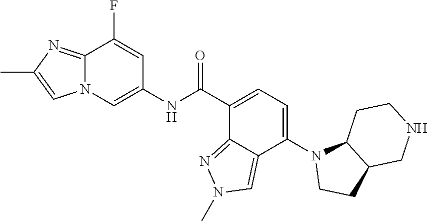 [SMILES: Cc1cn2cc(NC(=O)c3ccc(N4CC[C@@H]5CNCC[C@@H]54)c4cn(C)nc34)cc(F)c2n1] Compound 101 | 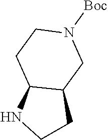 [SMILES: CC(C)(C)OC(=O)N1CC[C@@H]2NCC[C@@H]2C1] | Modification: Step 1 was ran using RuPhos and Pd₂(dba)₃. The crude product was purified by Prep- HPLC (Condition 1, Gradient 1) to afford N-(8-fluoro-2-methyl-imidazo[1,2-a]- pyridin-6-yl)-2-methyl-4-(octahydro- pyrrolo[3,2-c]-pyridin-1-yl)indazole-7- carboxamide hydrochloride (Compound 101, 83.4 mg). LCMS (ES, m/z): 448 [M + H]⁺. ¹H NMR (400 MHz, DMSO-d₆) δ 11.34 (s, 1H), 9.61 (d, J = 1.5 Hz, 1H), 9.45-9.44 (m, J = 11.2 Hz, 1H), 9.16-9.15 (m, J = 11.5 Hz, 1H), 8.89 (s, 1H), 8.29 (d, J = 2.3 Hz, 1H), 8.12 (dd, J = 11.8, 1.6 Hz, 1H), 7.95 (d, J = 8.3 Hz, 1H), 6.18 (d, J = 8.5 Hz, 1H), 4.41-4.31 (m, 1H), 4.30 |
| | | (s, 3H), 3.84 (d, J = 9.2 Hz, 1H), 3.70 (q, |
| | | J = 8.8 Hz, 1H), 3.38-3.37 (m, J = 13.4 |
| | | Hz, 1H), 3.24 (dd, J = 29.2, 12.8 Hz, 2H), |
| | | 3.06 (q, J = 12.1 Hz, 1H), 2.61 (ddd, J = |
| | | 28.5, 12.8, 7.8 Hz, 2H), 2.25 (d, J = 13.5 |
| | | Hz, 1H), 2.14-2.05 (m, 1H), 1.69 (q, J = |
| | | 11.2 Hz, 1H). |
| 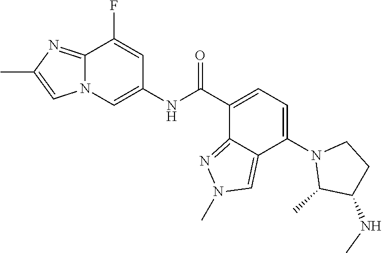 [SMILES: CN[C@H]1CCN(c2ccc(C(=O)Nc3cc(F)c4nc(C)cn4c3)c3nn(C)cc23)[C@H]1C] Compound 152 | 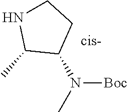 A110 | Modifications: Step 1 was ran for 2 h at 110° C. Step 2 was ran in dioxane. The resulting mixture was concentrated under reduced pressure and purified by Prep-Chiral-HPLC (Condition 2, Gradient 1) to afford N-(8-fluoro-2- methylimidazo-[1,2-a]pyridin-6-yl)-2- methyl-4-[(2S,3S)-2-methyl-3- (methylamino)pyrrolidin-1-yl]indazole- 7-carboxamide (Compound 152, 25 mg, 57.4 μmol). Absolute stereochemistry was assigned arbitrarily. LCMS (ES, m/z): 436 [M + H]⁺. ¹H NMR (400 MHz, DMSO-d₆) δ 11.05 (s, 1H), 9.20 (d, J = 1.6 Hz, 1H), 8.80 (s, 1H), 7.95 (d, J = 8.3 Hz, 1H), 7.89 (d, J = 3.1 Hz, 1H), 7.32 (dd, J = 12.3, 1.6 Hz, 1H), 6.11 (d, J = 8.5 Hz, 1H), 4.46 (q, J = 6.9 Hz, 1H), 4.29 (s, |
| | | 3H), 3.85 (t, J = 9.4 Hz, 1H), 3.66 (d, J = |
| | | 9.6 Hz, 1H), 3.58 (s, 1H), 2.92 (q, J = 7.3 |
| | | Hz, 1H), 2.58 (s, 3H), 2.40 (s, 1H), 2.35 |
| | | (s, 3H), 2.09 (s, 1H), 1.29-1.13 (m, 4H). |
| 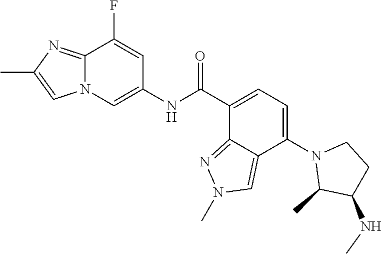 Compound 153 | 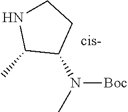 A110 | Modifications: Step 1 was ran for 2 h at 110° C. Step 2 was ran in dioxane. The product was purified by Prep-Chiral- HPLC (Condition 2, Gradient 1) to afford N-(8-fluoro-2-methyl- imidazo[1,2-a]pyridin-6-yl)-2-methyl-4- [(2R,3R)-2-methyl-3- (methylamino)pyrrolidin-1-yl]-indazole- 7-carboxamide (Compound 153, 60 mg, 137.8 μmol). Absolute stereochemistry was assigned arbitrarily. LCMS (ES, m/z): 436 [M + H]⁺. ¹H NMR (400 MHz, DMSO-d₆) δ 11.04 (s, 1H), 9.20 (d, J = 1.6 Hz, 1H), 8.79 (s, 1H), 7.96-7.86 (m, 2H), 7.31 (dd, J = 12.4, 1.7 Hz, 1H), 6.08 (d, J = 8.5 Hz, 1H), 4.35 (q, J = 6.5, 5.5 Hz, 1H), 4.28 (s, 3H), 3.74 (t, J = 9.8 Hz, 1H), 3.59 (q, J = 9.5 Hz, 1H), 3.21 (dt, J = |
| | | 12.4, 6.5 Hz, 1H), 2.38 (s, 3H), 2.35 (s, |
| | | 3H), 2.21 (dt, J = 12.9, 6.8 Hz, 1H), 1.86 |
| | | (t, J = 10.8 Hz, 1H), 1.11 (d, J = 6.3 Hz, |
| | | 3H). |
| 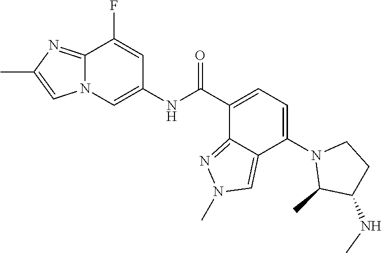 [SMILES: CN[C@H]1CCN(c2ccc(C(=O)Nc3cc(F)c4nc(C)cn4c3)c3nn(C)cc23)[C@@H]1C] Compound 154 | 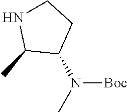 A156 | Modifications: Step 1 was ran for 2 h at 110° C. Step 2 was ran using TFA. The crude product was purified by Prep- HPLC (Condition 5, Gradient 5) to afford the product. The product was purified by Prep-Chiral-HPLC (Condition 2, Gradient 1) to afford N-(8-fluoro-2- methylimidazo-[1,2-a]pyridin-6-yl)-2- methyl-4-[(2R,3S)-2-methyl-3- (methylamino)pyrrolidin-1-yl]indazole- 7-carboxamide (Compound 154, 37 mg, 84.5 μmol). Absolute stereochemistry was assigned arbitrarily. LCMS (ES, m/z): 436 [M + H]⁺. ¹H NMR (400 MHz, DMSO-d₆) δ 11.04 (s, 1H), 9.20 (d, J = 1.6 Hz, 1H), 8.79 (s, 1H), 7.95-7.86 (m, 2H), 7.31 (dd, J = 12.3, 1.7 Hz, 1H), 6.06 (d, J = 8.4 Hz, 1H), 4.28 (s, 3H), 4.06 (d, |
| | | J = 6.6 Hz, 1H), 3.75 (dd, J = 14.8, 8.3 |
| | | Hz, 2H), 2.94 (d, J = 4.9 Hz, 1H), 2.33 (d, |
| | | J = 16.7 Hz, 7H), 1.99-1.89 (m, 1H), |
| | | 1.85 (s, 1H), 1.23 (d, J = 6.4 Hz, 3H). |
| 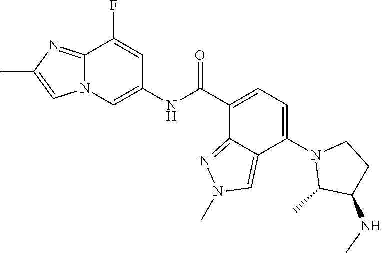 Compound 155 | 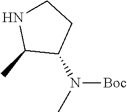 A156 | Modifications: Step 1 was ran for 2 h at 110° C. Step 2 was ran using TFA. The crude product was purified by Prep- HPLC (Condition 5, Gradient 5) to afford the product. The product was purified by Prep-Chiral-HPLC (Condition 2, Gradient 1) to afford N-(8-fluoro-2- methylimidazo-[1,2-a]pyridin-6-yl)-2- methyl-4-[(2S,3R)-2-methyl-3- (methylamino)-pyrrolidin-1-yl]indazole- 7-carboxamide (Compound 155, 85 mg, 194 μmol). LCMS (ES, m/z): 436 [M + H]⁺. ¹H NMR (400 MHz, DMSO-d₆) δ 11.04 (s, 1H), 9.20 (d, J = 1.6 Hz, 1H), 8.79 (s, 1H), 7.95-7.86 (m, 2H), 7.31 (dd, J = 12.3, 1.7 Hz, 1H), 6.06 (d, J = 8.4 Hz, 1H), 4.28 (s, 3H), 4.06 (d, J = 6.6 Hz, 1H), 3.75 (dd, J = 14.8, 8.3 Hz, 2H), 2.94 |
| | | (d, J = 4.9 Hz, 1H), 2.33 (d, J = 16.7 Hz, |
| | | 7H), 1.99-1.89 (m, 1H), 1.85 (s, 1H), |
| | | 1.23 (d, J = 6.4 Hz, 3H). |
| 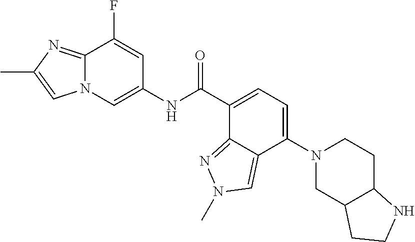 [SMILES: Cc1cn2cc(NC(=O)c3ccc(N4CCC5NCCC5C4)c4cn(C)nc34)cc(F)c2n1] Compound 169 | 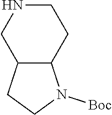 [SMILES: CC(C)(C)OC(=O)N1CCC2CNCCC21] | Modification: Step 1 was ran for 2 h. The crude product was purified by Prep- HPLC (Condition 2, Gradient 1) to afford N-(8-fluoro-2-methyl-imidazo[1,2-a]- pyridin-6-yl)-2-methyl-4-(octahydro- pyrrolo[3,2-c]pyridin-5-yl)indazole-7- carboxamide 2,2,2-trifluoroacetate (Compound 169, 147.5 mg). LCMS (ES, m/z): 448 [M + H]⁺. ¹H NMR (400 MHz, DMSO-d₆) δ 11.22 (s, 1H), 9.42 (s, 1H), 9.16 (s, 1H), 8.80 (s, 1H), 8.76 (s, 1H), 8.11 (s, 1H), 7.99 (d, J = 8.0 Hz, 1H), 7.77 (d, J = 12.0 Hz, 1H), 6.46 (d, J = 8.3 Hz, 1H), 3.87 (d, J = 7.9 Hz, 1H), 3.79-3.64 (m, 2H), 3.55 (dd, J = 13.3, 7.4 Hz, 1H), 3.43 (d, J = 11.1 Hz, 1H), 3.39- 3.38 (m, 1H), 3.26 (s, 1H), 2.65 (q, J = 6.6 |
| | | Hz, 1H), 2.44 (s, 3H), 2.21-2.11 (m, 2H), |
| | | 2.01-1.98 (m, 1H), 1.88 (dt, J = 13.6, 7.7 |
| | | Hz, 1H). |
| 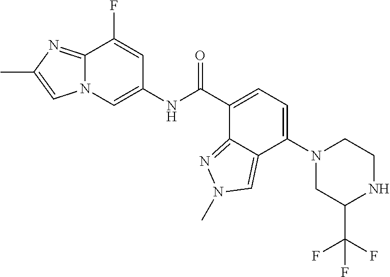 [SMILES: Cc1cn2cc(NC(=O)c3ccc(N4CCNC(C(F)(F)F)C4)c4cn(C)nc34)cc(F)c2n1] Compound 248 | 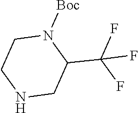 [SMILES: CC(C)(C)OC(=O)N1CCNCC1C(F)(F)F] | Modifications: Step 1 was ran for 2 h at 90° C. Step 2 was ran using TFA. The crude product was purified by Prep- HPLC (Condition 4, Gradient 1) to afford N-(8-fluoro-2-methyl-imidazo[1,2-a]- pyridin-6-yl)-2-methyl-4-[3-(trifluoro- methyl)piperazin-1-yl]indazole-7- carboxamide (Compound 248, 18.7 mg, 39.3 μmol). LCMS (ES, m/z): 476 [M + H]⁺. ¹H NMR (400 MHz, DMSO-d₆) δ 11.06 (s, 1H), 9.22 (s, 1H), 8.78 (s, 1H), 8.00 (d, J = 8.0 Hz, 1H), 7.91 (d, J = 3.0 Hz, 1H), 7.35 (d, J = 12.4 Hz, 1H), 6.58 (d, J = 8.1 Hz, 1H), 4.32 (s, 3H), 3.80 (d, J = 11.8 Hz, 1H), 3.74 (d, J = 11.7 Hz, 1H), 3.66 (s, 1H), 3.08 (dd, J = 11.9, 9.0 Hz, 4H), 2.97 (d, J = 11.7 Hz, 1H), 2.36 (s, 3H). |
| 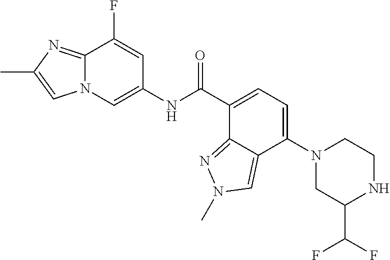 [SMILES: Cc1cn2cc(NC(=O)c3ccc(N4CCNC(C(F)F)C4)c4cn(C)nc34)cc(F)c2n1] Compound 249 | 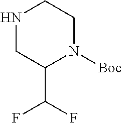 [SMILES: CC(C)(C)OC(=O)N1CCNCC1C(F)F] | Modifications: Step 1 was ran at 90° C. for 1 h. Step 2 was ran using TFA for 1 h. The crude product was purified by reversed-phase flash chromatography (Condition 1, Gradient 10) to afford 4-[3- (difluoromethyl)piperazin-1-yl]-N-(8- fluoro-2-methylimidazo[1,2-a]pyridin-6- yl)-2-methyl-indazole-7-carboxamide (Compound 249, 34 mg, 74.3 μmol). LCMS (ES, m/z): 458 [M + H]⁺. ¹H NMR (300 MHz, DMSO-d₆) δ 11.07 (s, 1H), 9.21 (d, J = 1.7 Hz, 1H), 8.76 (s, 1H), 8.00 (d, J = 8.0 Hz, 1H), 7.91 (dd, J = 3.2, 1.0 Hz, 1H), 7.35 (dd, J = 12.4, 1.7 Hz, 1H), 6.55 (d, J = 8.1 Hz, 1H), 6.06 (d, J = 4.5 Hz, 1H), 4.32 (s, 3H), 3.74 (d, J = 10.9 Hz, 2H), 3.24 (d, J = 11.0 Hz, 1H), 3.14- 2.86 (m, 4H), 2.36 (d, J = 0.8 Hz, 3H). |
| 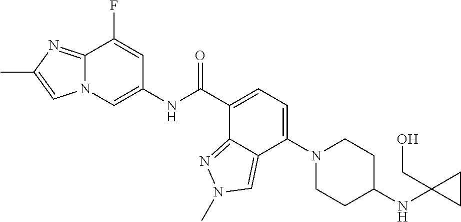 [SMILES: Cc1cn2cc(NC(=O)c3ccc(N4CCC(NC5(CO)CC5)CC4)c4cn(C)nc34)cc(F)c2n1] Compound 254 | 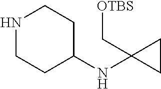 [SMILES: CC(C)(C)[Si](C)(C)OCC1(NC2CCNCC2)CC1] C105 | Modification: Step 1 was ran using RuPhos and Pd₂(dba)₃, for 2 h. The resulting mixture was purified by reversed-phase flash chromatography (Condition 3, Gradient 15) to afford N- (8-fluoro-2-methylimidazo[1,2- a]pyridin-6-yl)-4-[4-[[1- (hydroxymethyl)-cyclopropyl]amino]-1- piperidyl]-2-methylindazole-7- carboxamide (Compound 354) as a solid. LCMS (ES, m/z): 492 [M + H]⁺. ¹H NMR (400 MHz, DMSO-d₆) δ 11.07 (s, 1H), 9.22 (d, J = 1.6 Hz, 1H), 8.78 (s, 1H), 7.96 (d, J = 8.2 Hz, 1H), 7.91 (d, J = 3.0 Hz, 1H), 7.38-7.30 (m, 1H), 6.48 (d, J = 8.3 Hz, 1H), 4.53 (t, J = 5.5 Hz, 1H), |
| | | 4.30 (s, 3H), 3.87 (d, J = 12.5 Hz, 2H), |
| | | 3.38-3.35 (m, 3H), 3.06 (t, J = 11.7 Hz, |
| | | 3H), 2.35 (s, 3H), 1.95 (d, J = 12.5 Hz, |
| | | 2H), 1.47-1.38 (m, 2H), 0.43-0.42 (m, |
| | | 4H). |

### Example 3: Synthesis of Compound 170

### Synthesis of Intermediate A12

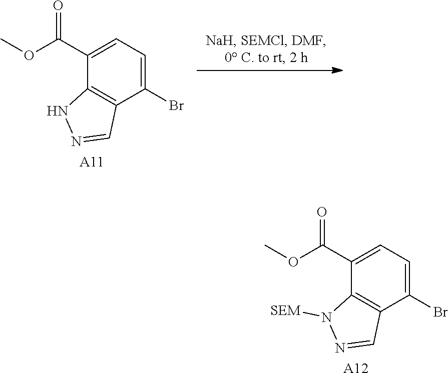

To a stirred solution of methyl 4-bromo-1H-indazole-7-carboxylate (A11, 2 g, 7.84 mmol, 1 eq) in DMF (20 mL) was added NaH (0.38 g, 15.68 mmol, 2 eq) in portions, and the reaction was stirred for 0.5 h at 0° C. under N₂, before dropwise addition of SEMCl (1.96 g, 11.76 mmol, 1.5 eq). The resulting mixture was stirred for additional 2 h at room temperature, quenched by the addition of acetic acid (4 mL) at 5° C., then basified to pH 8 with saturated NaHCO₃ (aq.), and later extracted with EtOAc (3×50 mL). The combined organic layers were washed with brine (40 mL), dried over anhydrous Na₂SO₄, and filtered. The filtrate was concentrated under reduced pressure and purified by silica gel column chromatography (50% THF in PE) to afford methyl 4-bromo-1-([2-(trimethylsilyl)ethoxy]methyl)indazole-7-carboxylate (A12, 1.2 g). LCMS (ES, m/z): 385 [M+H].

### Synthesis of Intermediate A13

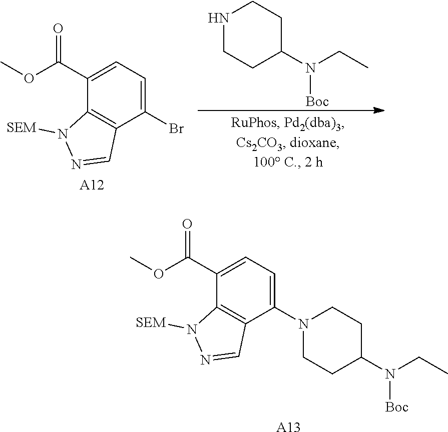

To a stirred solution of Intermediate A12 (700 mg, 1.82 mmol, 1 eq), Cs₂CO₃ (1.18 g, 3.63 mmol, 2 eq) and tert-butyl N-ethyl-N-(piperidin-4-yl)carbamate (498 mg, 2.18 mmol, 1.2 eq) in dioxane (20 mL) were added RuPhos (170 mg, 0.36 mmol, 0.2 eq) and Pd₂(dba)₃ (167 mg, 0.18 mmol, 0.1 eq), and the reaction was stirred for 2 h at 100° C. under N₂. The resulting mixture was allowed to cool to room temperature, quenched by the addition of water (50 mL), and extracted with DCM (3×20 mL). The combined organic layers were washed with brine (30 mL), dried over anhydrous Na₂SO₄, and filtered. The filtrate was concentrated under reduced pressure and purified by silica gel column chromatography (9% EtOAc in PE) to afford methyl 4-(4-[(tert-butoxycarbonyl)(ethyl)amino]piperidin-1-yl)-1-([2-(trimethylsilyl)ethoxy]methyl)indazole-7-carboxylate (A13, 500 mg). LCMS (ES, m/z): 533 [M+H].

### Synthesis of Intermediate A14

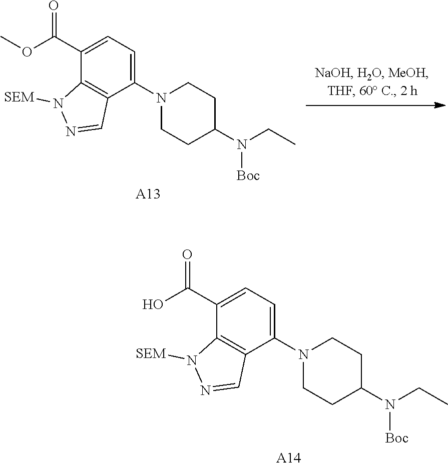 [SMILES: CCN(C(=O)OC(C)(C)C)C1CCN(c2ccc(C(=O)OC)c3c2cnn3COCC[Si](C)(C)C)CC1 ; "A13" ; CCN(C(=O)OC(C)(C)C)C1CCN(c2ccc(C(=O)O)c3c2cnn3COCC[Si](C)(C)C)CC1 ; "A14"]

To a stirred solution of Intermediate A13 (288 mg, 0.54 mmol, 1 eq) in MeOH (4 mL) and THF (4 mL) was added NaOH (65 mg, 1.62 mmol, 3 eq) in H₂O (2 mL) dropwise, and the reaction was stirred for 2 h at 60° C. The resulting mixture was allowed to cool to room temperature and concentrated under reduced pressure. The resulting mixture was diluted with DCM (10 mL), acidified to pH 5 with 1 M HCl (aq.), and later extracted with DCM (3×20 mL). The combined organic layer was washed with brine (30 mL), dried over anhydrous Na₂SO₄, filtered, and concentrated under reduced pressure to afford 4-(4-[(tert-butoxycarbonyl)(ethyl)amino]piperidin-1-yl)-1-([2-(trimethylsilyl)ethoxy]methyl)-indazole-7-carboxylic acid (A14, 240 mg). LCMS (ES, m/z): 519 [M+H].

### Synthesis of Intermediate A15

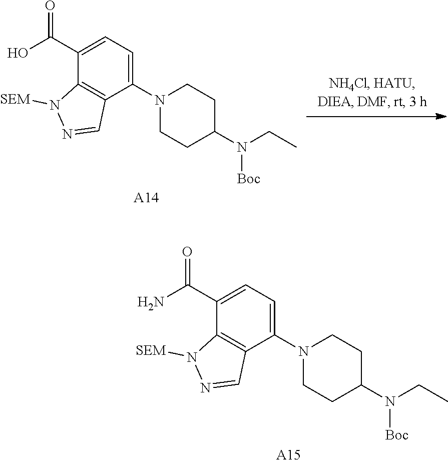 [SMILES: CCN(C(=O)OC(C)(C)C)C1CCN(c2ccc(C(=O)O)c3c2cnn3COCC[Si](C)(C)C)CC1 ; "A14" ; CCN(C(=O)OC(C)(C)C)C1CCN(c2ccc(C(N)=O)c3c2cnn3COCC[Si](C)(C)C)CC1 ; "A15"]

To a stirred solution of Intermediate A14 (240 mg, 0.46 mmol, 1 eq) and NH₄Cl (37 mg, 0.7 mmol, 1.5 eq) in DMF (5 mL) were added HATU (264 mg, 0.70 mmol, 1.5 eq) and DIEA (180 mg, 1.39 mmol, 3 eq) at room temperature. The resulting mixture was stirred for 3 h and then quenched by the addition of water (40 mL). The precipitated solids were collected by filtration and washed with water (3×5 mL) to afford tert-butyl N-[1-(7-carbamoyl-1-([2-(trimethylsilyl)-ethoxy]methyl)indazol-4-yl)piperidin-4-yl]-N-ethylcarbamate (A15, 205 mg). LCMS (ES, m/z): 518 [M+H].

### Synthesis of Intermediate A16

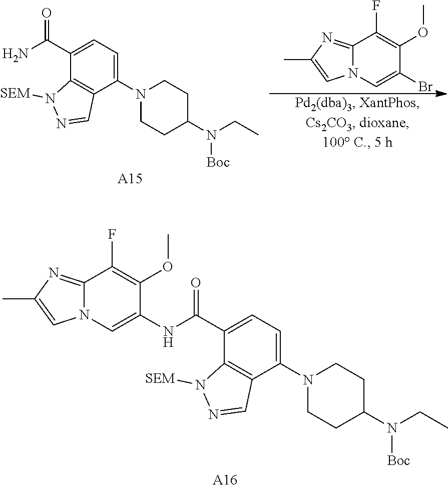 [SMILES: CCN(C(=O)OC(C)(C)C)C1CCN(c2ccc(C(N)=O)c3c2cnn3COCC[Si](C)(C)C)CC1 ; COc1c(Br)cn2cc(C)nc2c1F ; "A15" ; CCN(C(=O)OC(C)(C)C)C1CCN(c2ccc(C(=O)Nc3cn4cc(C)nc4c(F)c3OC)c3c2cnn3COCC[Si](C)(C)C)CC1 ; "A16"]

To a stirred solution of Intermediate A15 (205 mg, 0.40 mmol, 1 eq), Cs₂CO₃ (258 mg, 0.79 mmol, 2 eq) and 6-bromo-8-fluoro-7-methoxy-2-methylimidazo[1,2-a]pyridine (123 mg, 0.48 mmol, 1.2 eq) in dioxane (10 mL) were added XantPhos (46 mg, 0.08 mmol, 0.2 eq) and Pd₂(dba)₃ (36 mg, 0.04 mmol, 0.1 eq) at room temperature. The resulting mixture was stirred for 5 h at 100° C. under N₂, allowed to cool to room temperature, then quenched by the addition of water (50 mL) and later extracted with EtOAc (3×20 mL). The combined organic layers were washed with brine (30 mL), dried over anhydrous Na₂SO₄, and filtered. The filtrate was concentrated under reduced pressure and purified by reversed-phase flash chromatography (Condition 3, Gradient 1) to afford tert-butyl N-ethyl-N-(1-[7-((8-fluoro-7-methoxy-2-methylimidazo[1,2-a]pyridin-6-yl)carbamoyl)-1-([2-(trimethylsilyl)ethoxy]methyl)-indazol-4-yl]piperidin-4-yl)carbamate (A16, 100 mg). LCMS (ES, m/z): 696 [M+H].

### Synthesis of Compound 170

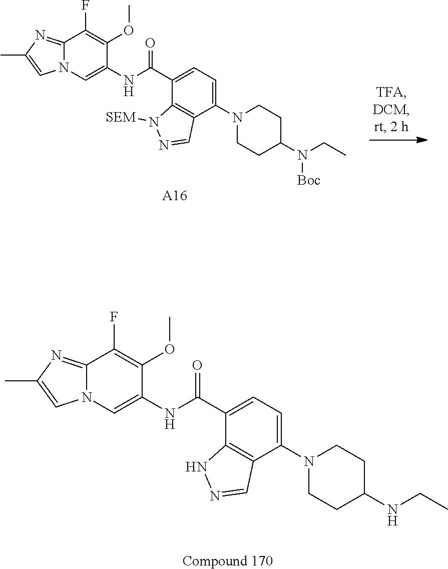

To a stirred solution of Intermediate A16 (100 mg, 0.14 mmol, 1 eq) in DCM (3 mL) was added TFA (1 mL) dropwise, and the reaction was stirred for 2 h at room temperature. The resulting mixture was concentrated under reduced pressure and basified to pH 8 with 7 M NH₃ (g) in MeOH. The resulting mixture was concentrated under reduced pressure and purified by reversed-phase flash chromatography (Condition 3, Gradient 2) to afford 4-[4-(ethylamino)piperidin-1-yl]-N-(8-fluoro-7-methoxy-2-methylimidazo[1,2-a]pyridin-6-yl)-1H-indazole-7-carboxamide (Compound 170, 20 mg). LCMS (ES, m/z): 466 [M+H]⁺. ¹H NMR (400 MHz, DMSO-d₆) δ 9.10 (s, 1H), 8.50 (s, 1H), 7.99 (d, J=8.1 Hz, 1H), 7.76 (d, J=3.0 Hz, 1H), 6.51 (d, J=8.2 Hz, 1H), 4.11 (s, 3H), 3.99-3.90 (m, 2H), 3.15-3.05 (m, 2H), 2.70 (dd, J=9.9, 5.5 Hz, 1H), 2.62 (q, J=7.0 Hz, 2H), 2.33 (s, 3H), 2.02-1.93 (m, 2H), 1.53-1.39 (m, 2H), 1.05 (t, J=7.1 Hz, 3H).

An analogous method was followed to obtain the following compounds.

| Compound | Starting Material | Characterization |
|---|---|---|
| 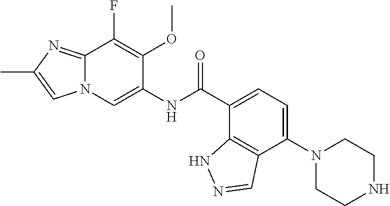 [SMILES: COc1c(NC(=O)c2ccc(N3CCNCC3)c3cn[nH]c23)cn2cc(C)nc2c1F] Compound 211 | 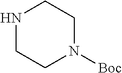 [SMILES: CC(C)(C)OC(=O)N1CCNCC1] | Modification: Step 4 was ran for 4 h. The crude product was purified by reversed-phase flash chromatography (Condition 1, Gradient 2) to afford N-(8- fluoro-7-methoxy-2-methylimidazo[1,2- a]pyridin-6-yl)-4-(piperazin-1-yl)-1H- indazole-7-carboxamide (Compound 211, 29 mg). LCMS (ES, m/z): 424 [M + H]⁺. ¹H NMR (300 MHz, DMSO-d₆) δ 9.13 (s, 1H), 8.54 (s, 1H), 8.01 (d, J = 8.0 Hz, 1H), 7.76 (d, J = 3.1 Hz, 1H), 6.51 (d, J = 8.2 Hz, 1H), 4.11 (s, 3H), 3.39 (t, J = 4.8 Hz, 4H), 2.93 (t, J = 4.9 Hz, 4H), 2.32 (s, 3H). |

### Example 4: Synthesis of Compound 171

### Synthesis of Intermediate A18

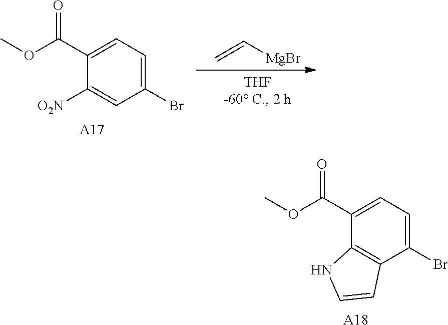

To a stirred mixture of methyl 4-bromo-2-nitrobenzoate (A17, 4 g, 15.38 mmol, 1 eq) in THF (40 mL) was added bromo (ethenyl) magnesium (56 mL, 168 mmol, 10.9 eq) dropwise at −70° C. under N₂. The resulting mixture was stirred for 2 h at −60° C., quenched by with saturated aqueous NH₄Cl (40 mL) at −60° C., and extracted with EtOAc (3×100 mL). The combined organic layers were washed with brine (2×30 mL), dried over anhydrous Na₂SO₄, and filtered. The filtrate was concentrated under reduced pressure and purified by silica gel column chromatography (4% EtOAc in PE) to afford methyl 4-bromo-1H-indole-7-carboxylate (A18, 0.9 g). LCMS (ES, m/z): 255 [M+H]⁺.

### Synthesis of Intermediate A19

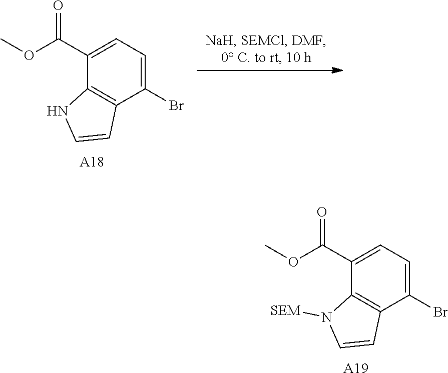

To a stirred solution of Intermediate A18 (2 g, 7.87 mmol, 1 eq) in THF (20 mL) was added NaH (0.63 g, 15.74 mmol, 2 eq, 60%) at 0° C. The resulting mixture was stirred for 30 min at 0° C., then SEMCl (1.97 g, 11.81 mmol, 1.5 eq) was dropwise at 0° C., and the resulting mixture was stirred for an additional 10 h at room temperature. The resulting mixture was quenched with saturated aqueous NH₄Cl (20 mL) and extracted with EtOAc (3×30 mL). The combined organics were washed with brine (30 mL), dried over anhydrous Na₂SO₄, and filtered. The filtrate was concentrated under reduced pressure and purified by silica gel column chromatography (100% PE) to afford methyl 4-bromo-1-([2-(trimethylsilyl)ethoxy]methyl)-indole-7-carboxylate (A19, 900 mg). LCMS (ES, m/z): 385 [M+H]⁺.

### Synthesis of Intermediate A20

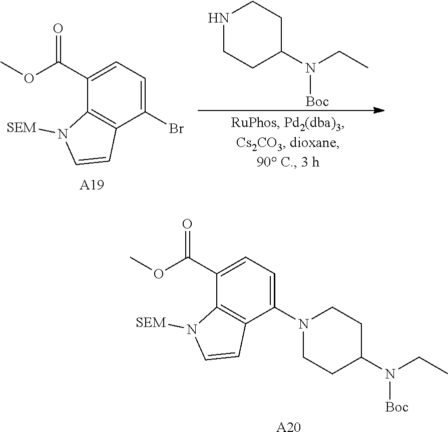

To a stirred solution of Intermediate A19 (450 mg, 1.17 mmol, 1 eq) and tert-butyl N-ethyl-N-(piperidin-4-yl)carbamate (320 mg, 1.41 mmol, 1.2 eq) in 1,4-dioxane (6 mL) were added Cs₂CO₃ (1.2 g, 3.51 mmol, 3 eq), RuPhos (109 mg, 0.23 mmol, 0.2 eq) and Pd₂(dba)₃ (107 mg, 0.12 mmol, 0.1 eq), and the reaction was stirred for 3 h 90° C. under N₂. The resulting mixture was concentrated under reduced pressure and purified by silica gel column chromatography (100% PE) to afford methyl 4-(4-[(tert-butoxycarbonyl)(ethyl)-amino]piperidin-1-yl)-1-([2-(trimethylsilyl)ethoxy]methyl)indole-7-carboxylate (A20, 405 mg). LCMS (ES, m/z): 532 [M+H]⁺.

### Synthesis of Intermediate A21

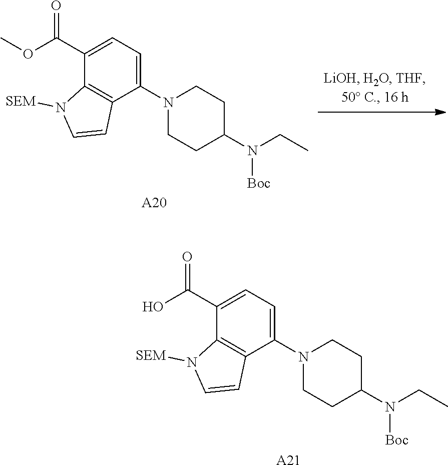 [SMILES: CCN(C(=O)OC(C)(C)C)C1CCN(c2ccc(C(=O)OC)c3c2ccn3COCC[Si](C)(C)C)CC1 ; "A20" ; CCN(C(=O)OC(C)(C)C)C1CCN(c2ccc(C(=O)O)c3c2ccn3COCC[Si](C)(C)C)CC1 ; "A21"]

To a stirred solution of Intermediate A20 (405 mg, 0.76 mmol, 1 eq) and LiOH (91 mg, 3.81 mmol, 5 eq) in MeOH (2 mL) were added THF (2 mL) and H₂O (1 mL) at room temperature. The resulting mixture was stirred for 16 h at 50° C., allowed to cool to room temperature and later concentrated under reduced pressure. The residue was adjusted pH 6 with 1 N HCl and extracted with EtOAc (3×5 mL). The combined organic layers were washed with brine (2×10 mL), dried over anhydrous Na₂SO₄, filtered, and concentrated under reduced pressure to afford 4-(4-[(tert-butoxycarbonyl)(ethyl)amino]piperidin-1-yl)-1-([2-(trimethylsilyl)-ethoxy]methyl)indole-7-carboxylic acid (A21, 360 mg). LCMS (ES, m/z): 518 [M+H]⁻.

### Synthesis of Intermediate A22

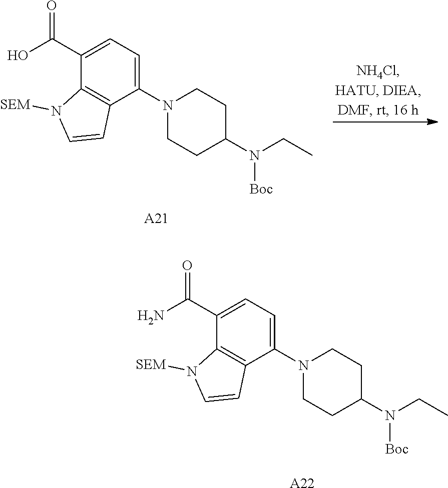 [SMILES: CCN(C(=O)OC(C)(C)C)C1CCN(c2ccc(C(=O)O)c3c2ccn3COCC[Si](C)(C)C)CC1 ; "A21" ; CCN(C(=O)OC(C)(C)C)C1CCN(c2ccc(C(N)=O)c3c2ccn3COCC[Si](C)(C)C)CC1 ; "A22"]

To a stirred solution of Intermediate A21 (200 mg, 0.39 mmol, 1 eq) and NH₄Cl (62 mg, 1.16 mmol, 3 eq) in DMF (6 mL) were added DIEA (150 mg, 1.16 mmol, 3 eq) and HATU (220 mg, 0.58 mmol, 1.5 eq) at room temperature. The resulting mixture was stirred for 16 h at room temperature and then quenched with water (20 mL). The resulting solid was collected by filtration, washed with water (2×10 mL), and dried under infrared light to afford tert-butyl N-[1-(7-carbamoyl-1-([2-(trimethylsilyl)ethoxy]methyl)indol-4-yl)piperidin-4-yl]-N-ethylcarbamate (A22, 170 mg). LCMS (ES, m/z): 517 [M+H]⁺.

### Synthesis of Intermediate A23

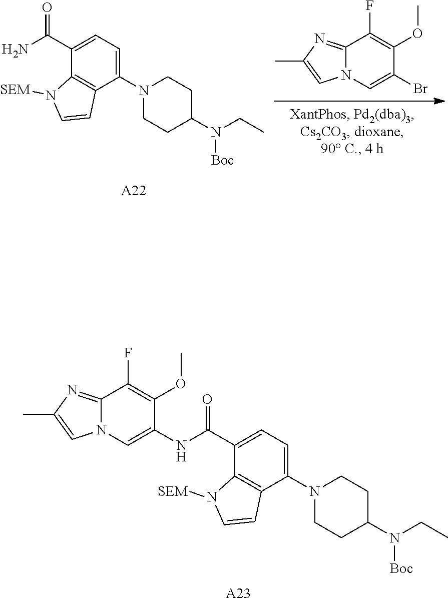

To a stirred solution of Intermediate A22 (170 mg, 0.33 mmol, 1 eq) and 6-bromo-8-fluoro-7-methoxy-2-methylimidazo[1,2-a]pyridine (128 mg, 0.49 mmol, 1.5 eq) in 1,4-dioxane (5 mL) were added Cs₂CO₃ (322 mg, 0.99 mmol, 3 eq), XantPhos (38 mg, 0.07 mmol, 0.2 eq) and Pd₂(dba)₃ (30 mg, 0.033 mmol, 0.1 eq), and the reaction was stirred for 4 h at 90° C. under N₂. The resulting mixture was allowed to cool to room temperature, concentrated under reduced pressure, and purified by silica gel column chromatography (50% EtOAc in PE) to afford tert-butyl N-ethyl-N-(1-[7-((8-fluoro-7-methoxy-2-methylimidazo[1,2-a]pyridin-6-yl)carbamoyl)-1-([2-(trimethylsilyl)ethoxy]methyl)indol-4-yl]piperidin-4-yl)carbamate (A23, 120 mg).

### Synthesis of Compound 171

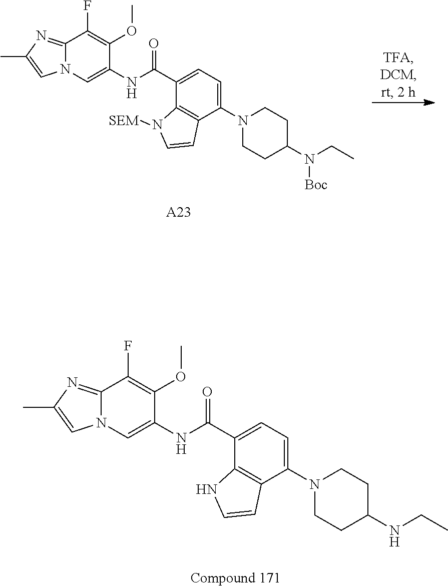

To a stirred solution of Intermediate A23 (120 mg, 0.17 mmol, 1 eq) in DCM (1.5 mL) was added TFA (0.5 mL), and the reaction was stirred for 2 h at room temperature. The resulting mixture was concentrated under reduced pressure and purified by reversed-phase flash chromatography (Condition 3, Gradient 3) to afford 4-[4-(ethylamino)piperidin-1-yl]-N-(8-fluoro-7-methoxy-2-methylimidazo[1,2-a]pyridin-6-yl)-1H-indole-7-carboxamide (Compound 171, 30 mg). LCMS (ES, m/z): 465 [M+H]⁺. ¹H NMR (300 MHz, DMSO-d₆) δ 11.08 (s, 1H), 9.54 (s, 1H), 8.69 (s, 1H), 7.83-7.70 (m, 2H), 7.26 (s, 1H), 6.56 (d, J=8.2 Hz, 1H), 6.46 (s, 1H), 4.01 (d, J=2.2 Hz, 3H), 3.76 (d, J=12.1 Hz, 2H), 2.90 (t, J=11.1 Hz, 2H), 2.64 (t, J=7.1 Hz, 3H), 2.34 (s, 3H), 1.97 (d, J=12.7 Hz, 2H), 1.50 (s, 2H), 1.05 (t, J=7.0 Hz, 3H).

### Example 5: Synthesis of Compound 172

### Synthesis of Intermediate A24

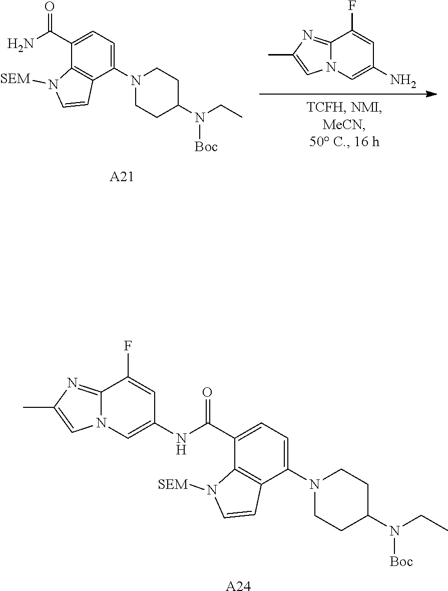 [SMILES: CCN(C(=O)OC(C)(C)C)C1CCN(c2ccc(C(N)=O)c3c2ccn3COCC[Si](C)(C)C)CC1 ; Cc1cn2cc(N)cc(F)c2n1 ; "TCFH, NMI,MeCN,50° C., 16 h" ; "A21" ; CCN(C(=O)OC(C)(C)C)C1CCN(c2ccc(C(=O)Nc3cc(F)c4nc(C)cn4c3)c3c2ccn3COCC[Si](C)(C)C)CC1 ; "A24"]

To a stirred solution of Intermediate A21 (200 mg, 0.39 mmol, 1 eq) and 8-fluoro-2-methylimidazo[1,2-a]pyridin-6-amine (95 mg, 0.58 mmol, 1.5 eq) in MeCN (4 mL) were added NMI (95 mg, 1.16 mmol, 3 eq) and TCFH (162 mg, 0.58 mmol, 1.5 eq), and the reaction was stirred for 16 h at 50° C. The resulting mixture was cooled to room temperature, concentrated under reduced pressure and purified by silica gel column chromatography (50% EtOAc in PE) to afford tert-butyl N-ethyl-N-(1-[7-((8-fluoro-2-methylimidazo[1,2-a]pyridin-6-yl)carbamoyl)-1-([2-(trimethylsilyl)ethoxy]methyl)indol-4-yl]-piperidin-4-yl)carbamate (A24, 90 mg). LCMS (ES, m/z): 665 [M+H]⁺.

### Synthesis of Compound 172

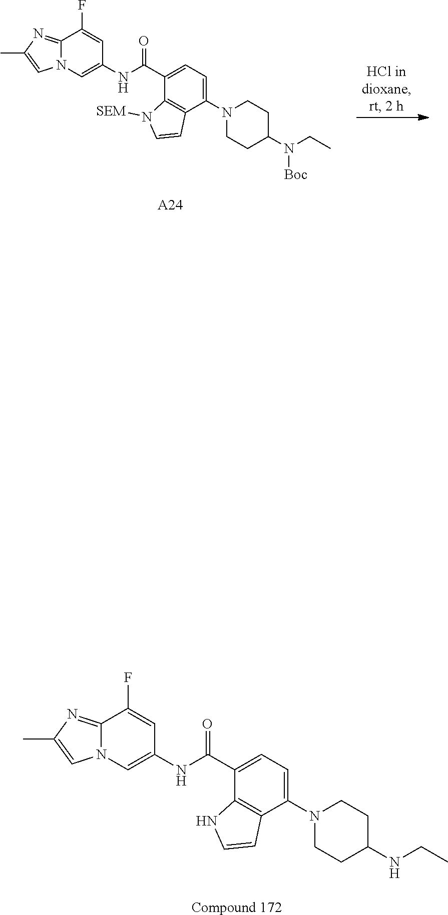

A mixture of Intermediate A24 (90 mg, 0.14 mmol, 1 eq) and 4 M HCl (gas) in 1,4-dioxane (5 mL) was stirred for 2 h at room temperature. The resulting mixture was concentrated under reduced pressure and purified by reversed-phase flash chromatography (Condition 4, Gradient 1) to afford 4-[4-(ethylamino)piperidin-1-yl]-N-(8-fluoro-2-methylimidazo[1,2-a]pyridin-6-yl)-1H-indole-7-carboxamide (Compound 172, 13 mg). LCMS (ES, m/z): 435 [M+H]⁺. ¹H NMR (300 MHz, DMSO-d₆) δ 11.14 (s, 1H), 10.10 (s, 1H), 9.07 (s, 1H), 7.90 (s, 1H), 7.79 (d, J=8.1 Hz, 1H), 7.41 (d, J=12.8 Hz, 1H), 7.27 (s, 1H), 6.57 (d, J=8.4 Hz, 1H), 6.47 (s, 1H), 3.76 (d, J=12.5 Hz, 2H), 2.88 (d, J=12.2 Hz, 2H), 2.69-2.58 (m, 3H), 2.36 (s, 3H), 1.96 (s, 2H), 1.50 (s, 2H), 1.05 (t, J=7.1 Hz, 3H).

An analogous method was followed to obtain the following compounds.

| Compound | Starting Material | Characterization |
|---|---|---|
| 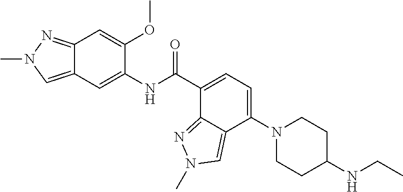 [SMILES: CCNC1CCN(c2ccc(C(=O)Nc3cc4cn(C)nc4cc3OC)c3nn(C)cc23)CC1] Compound 388 | 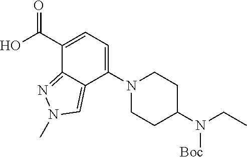 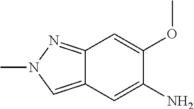 | Modification: Step 1 was ran for 16 h at room temperature. The resulting mixture were concentrated under reduced pressure and basified to pH 8 with saturated NaHCO₃ (aq.). The crude product was purified by Prep-HPLC (Condition 5, Gradient 8) to afford 4-[4- (ethylamino)-1-piperidyl]-N- (6-methoxy-2-methyl-indazol- 5-yl)-2-methyl-indazole-7- carboxamide (Compound 388, 10.3 mg, 22.3 umol, 16% yield) as a solid. LCMS (ES, m/z): 462 [M + H]⁺. ¹H NMR (400 MHz, DMSO-d₆) δ 11.61 (s, 1H), 8.84 (s, 1H), 8.73 (s, 1H), 8.18 (s, 1H), 7.99 (d, J = 8.0 Hz, 1H), 7.07 (s, 1H), 6.48 (d, J = |
| | | 8.2 Hz, 1H), 4.29 (s, 3H), 4.07 |
| | | (d, J = 14.3 Hz, 6H), 3.87 (d, J = |
| | | 12.4 Hz, 2H), 3.03 (t, J = 11.7 |
| | | Hz, 2H), 2.70-2.55 (m, 3H), |
| | | 1.96 (d, J = 12.6 Hz, 2H), 1.45 |
| | | (q, J = 9.9 Hz, 2H), 1.04 (t, J = |
| | | 7.1 Hz, 3H). |

### Example 6: Synthesis of Compound 104

### Synthesis of Intermediate A25

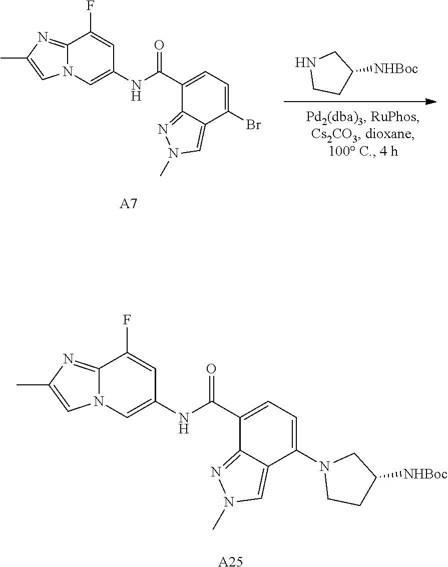

To a stirred mixture of Intermediate A7 (300 mg, 0.75 mmol, 1 eq) and tert-butyl N-[(3R)-pyrrolidin-3-yl]carbamate (165 mg, 0.9 mmol, 1.2 eq) in dioxane (6 mL) were added Cs₂CO₃ (730 mg, 2.24 mmol, 3 eq), RuPhos (70 mg, 0.15 mmol, 0.2 eq) and Pd₂(dba)₃ (68 mg, 0.075 mmol, 0.1 eq), and the reaction was stirred for 4 h at 100° C. under N₂. The resulting mixture was diluted with water (10 mL) and extracted with EtOAc (3×10 mL). The combined organic layers were washed with brine (3×20 mL), dried over anhydrous Na₂SO₄, and filtered. The filtrate was concentrated under reduced pressure and purified by silica gel column chromatography (89% EtOAc in PE) to afford tert-butyl N-[(3R)-1-[7-((8-fluoro-2-methylimidazo[1,2-a]pyridin-6-yl)carbamoyl)-2-methylindazol-4-yl]pyrrolidin-3-yl]carbamate (A25, 270 mg). LCMS (ES, m/z): 508 [M+H]⁺.

### Synthesis of Intermediate A26

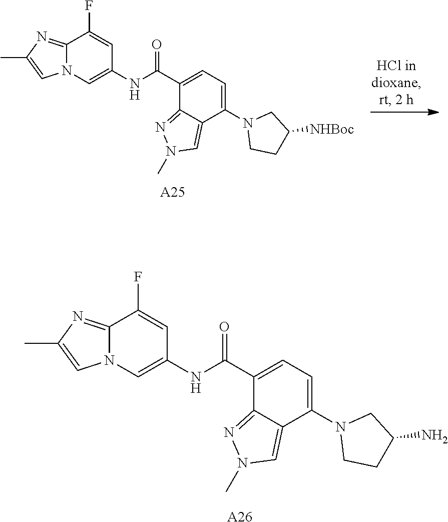

To a mixture of Intermediate A25 (270 mg, 0.53 mmol, 1 eq) in DCM (6 mL) was added HCl (gas) in 1,4-dioxane (2 mL, 65.83 mmol, 123.7 eq), and the reaction was stirred for 2 h at room temperature. The resulting mixture was concentrated under reduced pressure to afford 4-[(3R)-3-aminopyrrolidin-1-yl]-N-(8-fluoro-2-methylimidazo[1,2-a]pyridin-6-yl)-2-methylindazole-7-carboxamide (A26, 200 mg). LCMS (ES, m/z): 408 [M+H]⁺.

### Synthesis of Compound 104

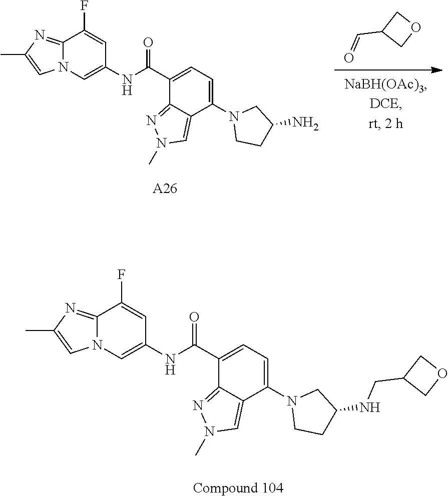

To a stirred solution of Intermediate A26 (240 mg, 0.59 mmol, 1 eq) and oxetane-3-carbaldehyde (61 mg, 0.71 mmol, 1.2 eq) in DCE (5 mL) was added NaBH(OAc)₃ (375 mg, 1.77 mmol, 3 eq), and the reaction was stirred for 2 h at room temperature. The resulting mixture was concentrated under reduced pressure and purified by reversed-phase flash chromatography (Condition 3, Gradient 4) to afford N-(8-fluoro-2-methylimidazo[1,2-a]pyridin-6-yl)-2-methyl-4-[(3R)-3-[(oxetan-3-ylmethyl)amino]pyrrolidin-1-yl]indazole-7-carboxamide (Compound 104, 40 mg). LCMS (ES, m/z): 478 [M+H]⁺. ¹H NMR (300 MHz, DMSO-d₆) δ 11.01 (s, 1H), 9.19 (d, J=1.6 Hz, 1H), 8.83 (s, 1H), 7.96-7.85 (m, 2H), 7.30 (d, J=12.5 Hz, 1H), 6.01 (d, J=8.5 Hz, 1H), 4.63 (td, J=6.0, 3.1 Hz, 2H), 4.30-4.28 (m, 5H), 3.81-3.80 (m, 1H), 3.72-3.71 (m, 1H), 3.63-3.62 (m, 1H), 3.43-3.41 (m, 2H), 3.08-2.97 (m, 1H), 2.88 (d, J=7.3 Hz, 2H), 2.34 (s, 3H), 2.15-2.13 (m, 1H), 1.92-1.90 (m, 1H).

### Example 7: Synthesis of Compound 107

### Synthesis of Intermediate A28

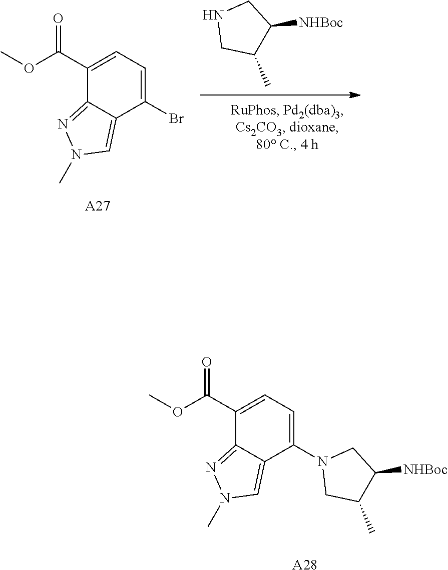

To a stirred mixture of methyl 4-bromo-2-methyl-indazole-7-carboxylate (A27, 250 mg, 929 μmol) and tert-butyl ((3S,4R)-4-methylpyrrolidin-3-yl)carbamate hydrochloride (242 mg, 1.02 mmol) in dioxane (5 mL) were added Cs₂CO₃ (606 mg, 1.86 mmol), RuPhos (87 mg, 185.8 μmol) and Pd₂(dba)₃ (85 mg, 92.9 μmol), and the reaction was stirred for 4 h at 80° C. under N₂. The resulting mixture was allowed to cool to room temperature, concentrated under reduced pressure, and purified by silica gel column chromatography (50% EtOAc in PE) to afford methyl 4-[(3S,4R)-3-(tert-butoxycarbonylamino)-4-methyl-pyrrolidin-1-yl]-2-methyl-indazole-7-carboxylate (A28, 260 mg, 669.3 μmol, 72% yield) as a solid. LCMS (ES, m/z): 389 [M+H]⁺.

### Synthesis of Intermediate A29

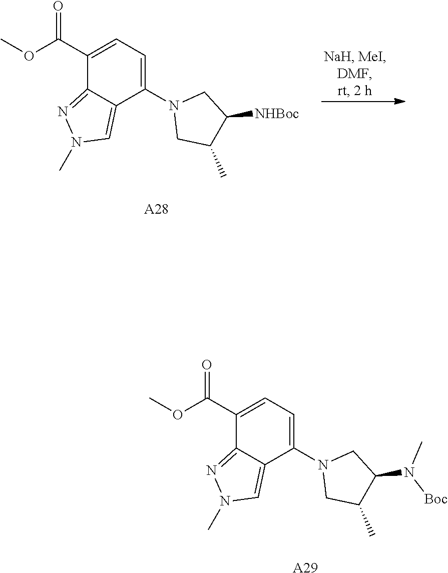 [SMILES: COC(=O)c1ccc(N2C[C@@H](C)[C@H](NC(=O)OC(C)(C)C)C2)c2cn(C)nc12 ; "NaH, MeI,DMF,rt, 2 h" ; "A28" ; COC(=O)c1ccc(N2C[C@@H](C)[C@H](N(C)C(=O)OC(C)(C)C)C2)c2cn(C)nc12 ; "A29"]

To a stirred mixture of Intermediate A28 (240 mg, 617.8 μmol) in DMF (3 mL) was added NaH (60% dispersion in oil, 30 mg, 1.24 mmol) in portions at 0° C. under N₂. The resulting mixture was stirred for 0.5 h, Iodomethane (176 mg, 1.24 mmol, 77 μL) was added dropwise, and the reaction was stirred for an additional 2 h at room temperature. The reaction was quenched by the addition of water (10 mL) and extracted with EtOAc (3×10 mL). The combined organic layers were washed with brine (20 mL), dried over anhydrous Na₂SO₄, and filtered. The filtrate was concentrated under reduced pressure and purified by silica gel column chromatography (50% EtOAc in PE) to afford methyl 4-[(3S,4R)-3-[tert-butoxycarbonyl(methyl)amino]-4-methyl-pyrrolidin-1-yl]-2-methyl-indazole-7-carboxylate (A29, 230 mg, 571.5 μmol). LCMS (ES, m/z): 403 [M+H]⁺.

### Synthesis of Intermediate A30

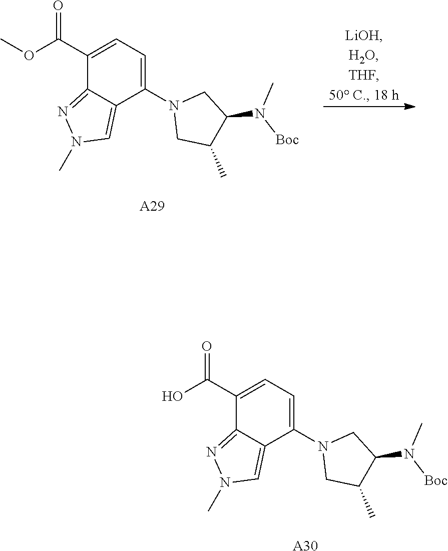 [SMILES: COC(=O)c1ccc(N2C[C@@H](C)[C@H](N(C)C(=O)OC(C)(C)C)C2)c2cn(C)nc12 ; "LiOH,H2O,THF,50° C., 18 h" ; "A29" ; C[C@@H]1CN(c2ccc(C(=O)O)c3nn(C)cc23)C[C@H]1N(C)C(=O)OC(C)(C)C ; "A30"]

To a stirred mixture of Intermediate A29 (210 mg, 521.8 μmol) in H₂O (1 mL) and THF (1 mL) was added LiOH (63 mg, 2.61 mmol) at room temperature. The resulting mixture was stirred for 18 h at 50° C., acidified to pH 5 with 1N HCl, and extracted with DCM (3×20 mL). The combined organic layers were washed with brine (10 mL), dried over anhydrous Na₂SO₄, filtered, and concentrated under reduced pressure to afford 4-[(3S,4R)-3-[tert-butoxycarbonyl-(methyl)amino]-4-methyl-pyrrolidin-1-yl]-2-methyl-indazole-7-carboxylic acid (A30, 160 mg, 411.9 μmol). LCMS (ES, m/z): 389 [M+H]⁺.

### Synthesis of Intermediate A31

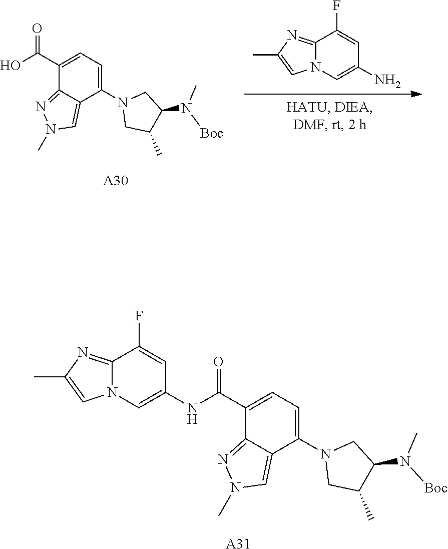

A mixture of Intermediate A30 (160 mg, 0.41 mmol, 1 eq), HATU (235 mg, 0.62 mmol, 1.5 eq) and DIEA (160 mg, 1.24 mmol, 3 eq) in DMF (4 mL) was stirred for 5 min at room temperature and then to the above mixture was added 8-fluoro-2-methylimidazo[1,2-a]pyridin-6-amine dihydrochloride (147 mg, 0.62 mmol, 1.5 eq) at room temperature, stirred for an additional 2 h, and then quenched with water (20 mL). The precipitated solids were collected by filtration and washed with water (3×5 mL) to afford tert-butyl ((3S,4R)-1-(7-((8-fluoro-2-methylimidazo[1,2-a]pyridin-6-yl)carbamoyl)-2-methyl-2H-indazol-4-yl)-4-methylpyrrolidin-3-yl)(methyl)carbamate (A31, 100 mg). LCMS (ES, m/z): 536 [M+H]⁺.

### Synthesis of Compound 107

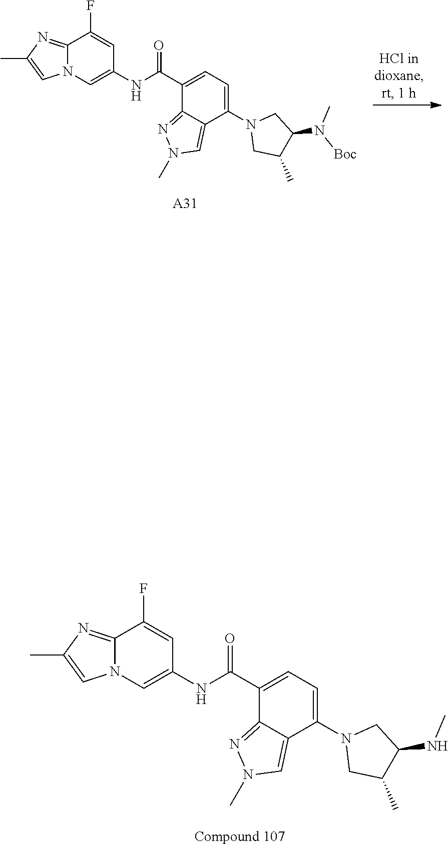

To a stirred mixture of Intermediate A31 (90 mg, 168.0 μmol) in DCM (5 mL) was added 4M HCl in dioxane (1 mL) and the reaction was stirred for 1 h at room temperature. The resulting mixture was concentrated under reduced pressure and purified by reverse flash chromatography (Condition 3, Gradient 3) to afford N-(8-fluoro-2-methyl-imidazo[1,2-a]pyridin-6-yl)-2-methyl-4-[(3R,4S)-3-methyl-4-(methylamino)pyrrolidin-1-yl]indazole-7-carboxamide (Compound 107, 19 mg, 43.6 μmol). LCMS (ES, m/z): 436 [M+H]⁺. Rt=2.32 min on chiral-SFC. ¹H NMR (400 MHz, DMSO-d₆) δ 11.01 (s, 1H), 9.20 (d, J=1.6 Hz, 1H), 8.84 (s, 1H), 7.96-7.86 (m, 2H), 7.31 (dd, J=12.4, 1.7 Hz, 1H), 6.01 (d, J=8.4 Hz, 1H), 4.27 (s, 3H), 3.87 (dd, J=14.9, 6.9 Hz, 2H), 3.39 (dd, J=10.5, 5.6 Hz, 1H), 2.90 (q, J=6.1 Hz, 1H), 2.40-2.33 (m, 6H), 2.22 (p, J=6.7 Hz, 1H), 1.10 (d, J=6.8 Hz, 3H).

An analogous method was followed to obtain the following compounds.

| Compounds | Starting Material | Characterization |
|---|---|---|
| 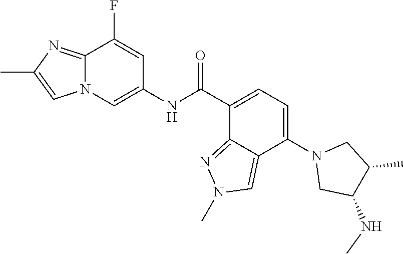 Compound 108 | 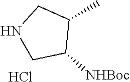 | The residue was purified by reverse flash chromatography (Condition 3, Gradient 3) to afford N-(8-fluoro-2-methyl- imidazo[1,2-a]pyridin-6-yl)-2-methyl-4- [(3S,4S)-3-methyl-4-(methylamino)- pyrrolidin-1-yl]indazole-7-carboxamide (Compound 108, 15.7 mg, 34.44 μmol). LCMS (ES, m/z): 436 [M + H].⁻ RT = 2.10 min on chiral-SFC. ¹H NMR (400 MHz, DMSO-d₆) δ 11.01 (s, 1H), 9.20 (d, J = 1.6 Hz, 1H), 8.84 (s, 1H), 7.96-7.86 (m, 2H), 7.31 (dd, J = 12.4, 1.7 Hz, 1H), 6.01 (d, J = 8.4 Hz, 1H), 4.27 (s, 3H), 3.87 (dd, J = 14.9, 6.9 Hz, 2H), 3.53-3.39 (m, 2H), 2.90 (q, J = 6.1 Hz, 1H), 2.35 (s, 7H), 1.10 (d, J = 6.8 Hz, 3H). |

### Example 8: Synthesis of A38, A42

### Synthesis of Intermediate A37

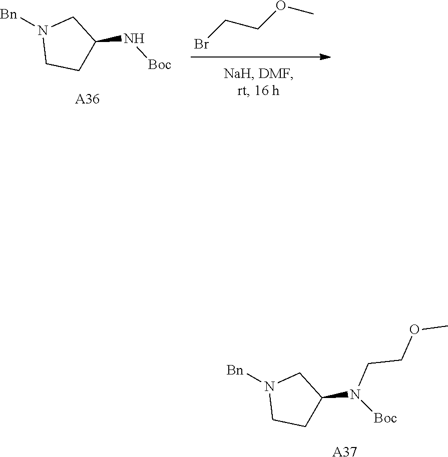

To a stirred solution of tert-butyl N-[(3S)-1-benzylpyrrolidin-3-yl]carbamate (A36, 5.0 g, 18.09 mmol) in DMF (60 mL) was added NaH (60% dispersion in oil; 651 mg, 27.14 mmol) in portions. The resulting mixture was stirred for 20 min at 0° C. under N₂, then 1-bromo-2-methoxyethane (3.02 g, 21.7 mmol, 2 mL) was added dropwise at 0° C., then the mixture was stirred for an additional 16 h at rt. The resulting mixture was quenched with saturated aqueous NH₄Cl (100 mL) and extracted with EtOAc (3×50 mL). The combined organic layers were washed with brine (30 mL), dried over anhydrous Na₂SO₄, and filtered. The filtrate was concentrated under reduced pressure and purified by silica gel column chromatography (5% EtOAc in PE) to afford tert-butyl N-[(3S)-1-benzylpyrrolidin-3-yl]-N-(2-methoxyethyl)carbamate (A37, 4.0 g, 11.96 mmol). LCMS (ES, m/z): 335 [M+H]⁺.

### Synthesis of Intermediate A38

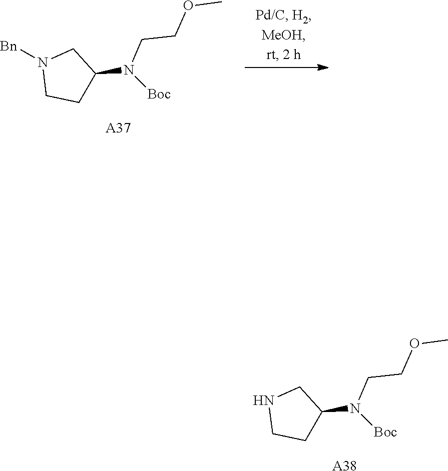 [SMILES: COCCN(C(=O)OC(C)(C)C)[C@H]1CCN(Cc2ccccc2)C1 ; "Pd/C, H2,MeOH,rt, 2 h" ; "A37" ; COCCN(C(=O)OC(C)(C)C)[C@H]1CCNC1 ; "A38"]

To a solution of Intermediate A37 (4.0 g, 11.96 mmol) in 40 mL MeOH was added Pd/C (1.53 g, 14.35 mmol, 10%). The mixture was hydrogenated at room temperature for 2 h under hydrogen atmosphere using a hydrogen balloon, filtered through a Celite pad and concentrated under reduced pressure to afford tert-butyl N-(2-methoxyethyl)-N-[(3S)-pyrrolidin-3-yl]carbamate (A38, 2.5 g, 10.23 mmol). LCMS (ES, m/z): 245 [M+H]⁺.

An analogous method was followed to obtain the following intermediates.

| Compound | Starting Material | Characterization |
|---|---|---|
| 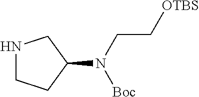 A42 | 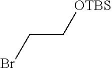 [SMILES: CC(C)(C)[Si](C)(C)OCCBr] | The reaction mixture was filtered through a Celite pad and concentrated under reduced pressure to afford tert-butyl N-(2-[(tert-butyldimethylsilyl)- oxy]ethyl)-N-[(3S)-pyrrolidin-3-yl]carbamate (A42, 650 mg). LCMS (ES, m/z): 345 [M + H]⁻. |

### Example 9: Synthesis of Compounds 112, 115

### Synthesis of Intermediate A40

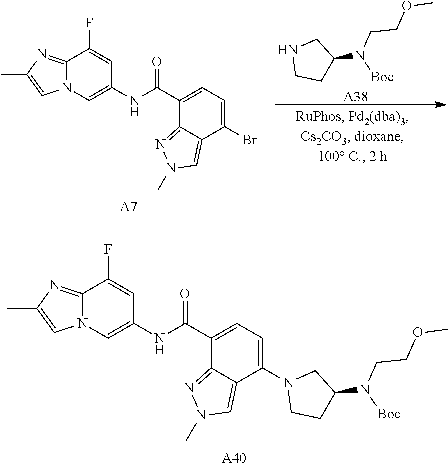

To a solution of 4-bromo-N-(8-fluoro-2-methyl-imidazo[1,2-a]pyridin-6-yl)-2-methyl-indazole-7-carboxamide (A7, 200 mg, 497.2 μmol) and A38 (146 mg, 596.7 μmol) in dioxane (2 mL) were added RuPhos (81 mg, 99.45 μmol), Pd₂(dba)₃ (46 mg, 49.72 μmol) and Cs₂CO₃ (486 mg, 1.49 mmol), and the reaction was stirred for 2 h at 100° C. under N₂. The resulting mixture was concentrated under reduced pressure and purified by silica gel column chromatography (50% EtOAc in PE) to afford tert-butyl N-[(3S)-1-[7-[(8-fluoro-2-methyl-imidazo[1,2-a]pyridin-6-yl)carbamoyl]-2-methyl-indazol-4-yl]pyrrolidin-3-yl]-N-(2-methoxyethyl)carbamate (A40, 150 mg, 265.2 μmol). LCMS (ES, m/z): 566 [M+H]⁺.

### Synthesis of Compound 112

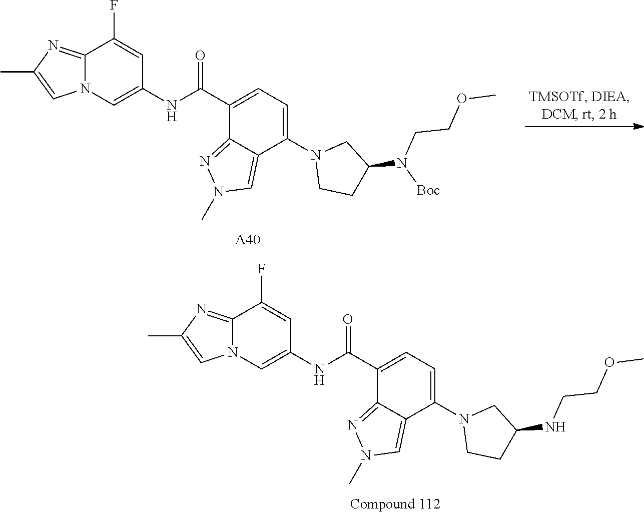

To a stirred solution of Intermediate A40 (150 mg, 265.2 μmol) in DCM (3 mL) was added 4M HCl (0.5 mL) dropwise at 0° C., and the reaction was stirred for 2 h at room temperature. The resulting mixture was concentrated under reduced pressure and purified by Prep-HPLC (Condition 3, Gradient 1) to afford N-(8-fluoro-2-methyl-imidazo[1,2-a]pyridin-6-yl)-4-[(3S)-3-(2-methoxyethylamino)pyrrolidin-1-yl]-2-methyl-indazole-7-carboxamide (Compound 112, 56.3 mg, 120.9 μmol). LCMS (ES, m/z): 466 [M+H]⁺. ¹H NMR (400 MHz, DMSO-d₆) δ 11.02 (s, 1H), 9.20 (d, J=1.7 Hz, 1H), 8.84 (s, 1H), 7.96-7.86 (m, 2H), 7.31 (dd, J=12.4, 1.7 Hz, 1H), 6.02 (d, J=8.4 Hz, 1H), 4.27 (s, 3H), 3.80 (d, J=9.0 Hz, 1H), 3.73 (d, J=8.0 Hz, 1H), 3.63 (d, J=7.5 Hz, 1H), 3.51-3.38 (m, 4H), 3.32-3.30 (m, 3H), 2.77 (t, J=5.7 Hz, 2H), 2.37-2.32 (m, 3H), 2.18 (dd, J=12.1, 5.9 Hz, 1H), 1.92 (dd, J=12.2, 6.4 Hz, 1H).

An analogous method was followed beginning at step 3, to obtain the following compounds.

| Compound | Starting Material | Characterization |
|---|---|---|
| 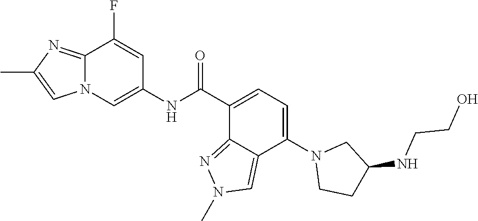 [SMILES: Cc1cn2cc(NC(=O)c3ccc(N4CC[C@H](NCCO)C4)c4cn(C)nc34)cc(F)c2n1] Compound 115 | 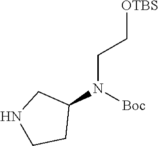 A42 | Modifications: Step 1 was ran at 80° C. for 6 h. Step 2 was ran using TFA in DCM. The resulting mixture was purified by reverse flash chromatography (Condition 3, Gradient 3) to afford N-(8-fluoro-2- methylimidazo-[1,2-a]-pyridin-6- yl)-4-[(3S)-3-(2-hydroxy- ethylamino)-pyrrolidin-1-yl]-2- methylindazole-7-carboxamide (Compound 115, 104.5 mg, 221.5 μmol). LCMS (ES, m/z): 452 [M + H]⁺. ¹H NMR (400 MHz, DMSO-d₆) δ 11.02 (s, 1H), 9.20 (d, J = 1.7 Hz, 1H), 8.84 (s, 1H), 7.93- |
| | | 7.89 (m, 2H), 7.31 (dd, J = 12.4, 1.7 |
| | | Hz, 1H), 6.02 (d, J = 8.4 Hz, 1H), |
| | | 4.53 (t, J = 5.3 Hz, 1H), 4.27 (s, 3H), |
| | | 3.92-3.70 (m, 2H), 3.64 (s, 1H), 3.49 |
| | | (t, J = 5.6 Hz, 4H), 2.69 (t, J = 5.8 |
| | | Hz, 2H), 2.35 (d, J = 0.8 Hz, 3H), |
| | | 2.17 (dt, J = 12.9, 6.5 Hz, 1H), 1.98- |
| | | 1.88 (m, 1H). |
| 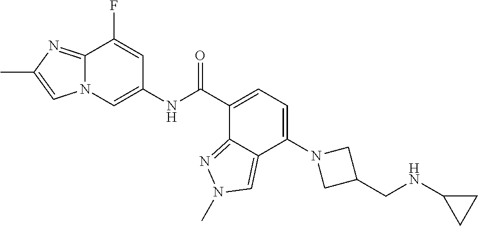 [SMILES: Cc1cn2cc(NC(=O)c3ccc(N4CC(CNC5CC5)C4)c4cn(C)nc34)cc(F)c2n1] Compound 298 | 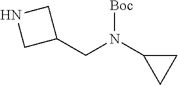 C39 | Modification: Step 3 was ran using RuPhos (0.2 eq) and RuPhos Pd G3 (0.1 eq). The last step was ran for 20 h. The crude product was purified by Prep-HPLC (Condition 13, Gradient 1) to afford 4-[3- [(cyclopropylamino)- methyl]azetidin-1-yl]-N-(8-fluoro- 2-methylimidazo[1,2-a]pyridin-6- yl)-2-methylindazole-7- carboxamide (Compound 298, 7.5 mg, 14% yield) as a solid. LCMS (ES, m/z): 448 [M + H]⁺. ¹H NMR (300 MHz, DMSO-d₆) δ 10.92 (s, 1H), 9.18 (d, J = 1.6 Hz, 1H), 8.65 (s, 1H), 7.94 (s, 1H), 7.90 (d, J = 5.0 |
| | | Hz, 1H), 7.31 (d, J = 12.8 Hz, 1H), |
| | | 5.86 (d, J = 8.2 Hz, 1H), 4.28 (d, J = |
| | | 7.4 Hz, 5H), 3.92 (s, 2H), 2.89 (s, |
| | | 3H), 2.35 (s, 3H), 2.12-2.04 (m, |
| | | 1H), 0.39 (d, J = 4.6 Hz, 2H), 0.22 |
| | | (s, 2H). |

### Example 10: Synthesis of Compounds 118 and 119

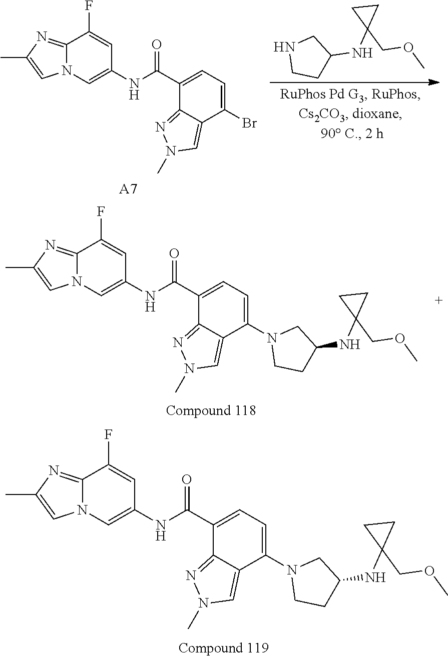

To a stirred solution of A7 (0.3 g, μmol) and N-[1-(methoxymethyl)cyclopropyl]pyrrolidin-3-amine (152.3 mg, 895.0 μmol) in dioxane (6 mL) were added Ruphos (69.6 mg, 149.2 μmol), Cs₂CO₃ (729.0 mg, 2.24 mmol) and RuPhos Pd G3 (62.3 mg, 74.59 μmol), and the reaction was stirred for 2 h at 90° C. under N₂. The resulting mixture was concentrated under reduced pressure and purified by silica gel column chromatography (90% EtOAc in PE) to afford a solid. The product was separated by Prep-HPLC (Condition 4, Gradient 1) to afford (S)—N-(8-fluoro-2-methylimidazo[1,2-a]pyridin-6-yl)-4-(3-((1-(methoxymethyl)cyclopropyl)amino)pyrrolidin-1-yl)-2-methyl-2H-indazole-7-carboxamide (Compound 118, 20.3 mg, 41.34 μmol) and (R)—N-(8-fluoro-2-methylimidazo[1,2-a]pyridin-6-yl)-4-(3-((1-(methoxymethyl)cyclopropyl)amino)-pyrrolidin-1-yl)-2-methyl-2H-indazole-7-carboxamide (Compound 119, 17.3 mg, 35.23 μmol). LCMS (ES, m/z): 492 [M+H]⁺. Absolute stereochemistry was assigned arbitrarily. Compound 118: RT=3.04 min on chiral-SFC. ¹H NMR (300 MHz, Methanol-d₄) δ 8.97 (d, J=1.6 Hz, 1H), 8.44 (s, 1H), 8.00 (d, J=8.3 Hz, 1H), 7.67 (d, J=2.9 Hz, 1H), 7.09 (dd, J=11.9, 1.6 Hz, 1H), 6.01 (d, J=8.4 Hz, 1H), 4.27 (s, 3H), 3.93-3.70 (m, 3H), 3.64-3.61 (m, 1H), 3.44-3.41 (m, 6H), 2.43 (s, 3H), 2.36-2.26 (m, 1H), 2.02-1.97 (m, 1H), 0.79-0.59 (m, 4H). Compound 119: RT=3.79 min on chiral-SFC. ¹H NMR (300 MHz, Methanol-d₄) δ 9.00 (t, J=1.8 Hz, 1H), 8.48 (d, J=2.5 Hz, 1H), 8.02 (d, J=8.3 Hz, 1H), 7.69 (d, J=3.1 Hz, 1H), 7.15 (d, J=11.8 Hz, 1H), 6.04 (d, J=8.5 Hz, 1H), 4.28 (s, 3H), 3.87-3.80 (m, 3H), 3.69-3.62 (m, 1H), 3.46-3.42 (m, 6H), 2.44 (s, 3H), 2.39-2.28 (m, 1H), 2.05-1.97 (m, 1H), 0.81-0.62 (m, 4H).

### Example 11: Synthesis of Compound 121

### Synthesis of Intermediate A44

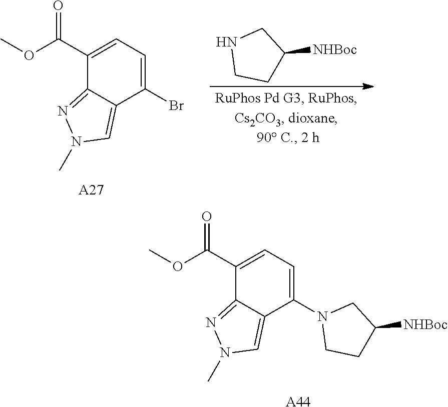

To a stirred mixture of Intermediate A27 (270 mg, 1.00 mmol) and tert-butyl N-[(3S)-pyrrolidin-3-yl]carbamate (224.3 mg, 1.2 mmol) in dioxane (7 mL) were added Cs₂CO₃ (326.9 mg, 1 mmol), RuPhos (93.5 mg, 200.4 μmol) and Pd₂(dba)₃ (91.9 mg, 100.3 μmol), and the reaction was stirred for 2 h at 90° C. under N₂. The resulting mixture was allowed to cool to room temperature, concentrated under reduced pressure, and purified by silica gel column chromatography (9% MeOH in DCM) to afford methyl 4-[(3S)-3-(tert-butoxycarbonylamino)pyrrolidin-1-yl]-2-methyl-indazole-7-carboxylate (A44, 330 mg, 881.3 μmol). LCMS (ES, m/z): 375 [M+H]⁺.

### Synthesis of Intermediate A45

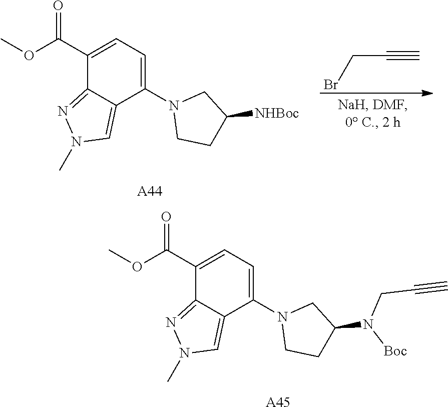

To a solution of Intermediate A44 (330 mg, 881.3 μmol) in DMF (6.6 mL) was added NaH (60% dispersion in oil; 42.3 mg, 1.76 mmol) at 0° C. The mixture was stirred for 15 min, then 3-bromoprop-1-yne (125.8 mg, 1.06 mmol) was added, the mixture was stirred for an additional 2 h at 0° C., then quenched by water (20 mL), and later extracted with EtOAc (3×20 mL). The combined organic layers were washed with water (3×50 mL), brine (50 mL), dried over anhydrous Na₂SO₄, and filtered. The filtrate was concentrated under reduced pressure and purified by silica gel column chromatography (9% MeOH in DCM) to afford methyl 4-[(3S)-3-[tert-butoxycarbonyl(prop-2-ynyl)amino]pyrrolidin-1-yl]-2-methyl-indazole-7-carboxylate (A45, 260 mg, 630.3 μmol). LCMS (ES, m/z): 413 [M+H]⁺.

### Synthesis of Intermediate A46

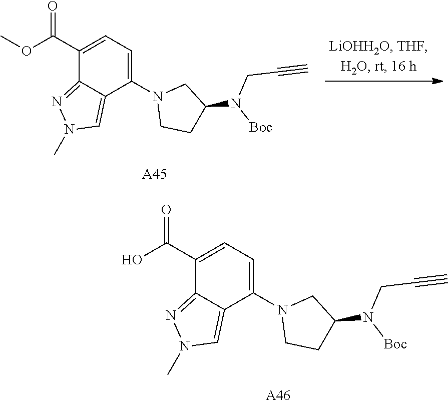

To a stirred mixture of Intermediate A45 (260 mg, 630.3 μmol) in THF (3 mL) were added H₂O (3 mL) and LiOH·H₂O (105.8 mg, 2.52 mmol), and the reaction was stirred for 16 h at room temperature. The resulting mixture was acidified to pH 6 with HCl (2N) and extracted with EtOAc (3×10 mL). The combined organic layers were washed with water (3×30 mL), dried over anhydrous Na₂SO₄, filtered, and concentrated under reduced pressure to afford 4-[(3S)-3-[tert-butoxycarbonyl(prop-2-ynyl)amino]pyrrolidin-1-yl]-2-methyl-indazole-7-carboxylic acid (A46, 240 mg, 602.3 μmol). LCMS (ES, m/z): 399 [M+H]⁺.

### Synthesis of Intermediate A47

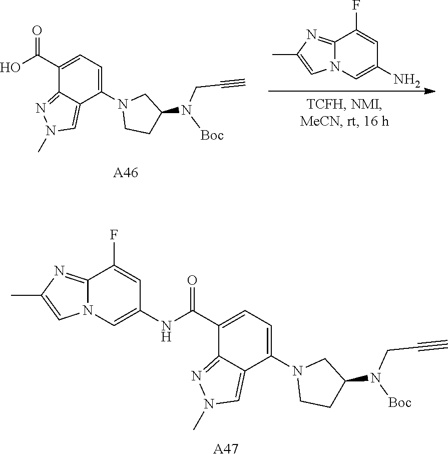

To a stirred mixture of Intermediate A46 (200 mg, 501.9 μmol) and 8-fluoro-2-methyl-imidazo[1,2-a]pyridin-6-amine dihydrochloride (99.5 mg, 602.3 μmol) in MeCN (4 mL) were added NMI (164.8 mg, 2.01 mmol, 159.3 μL) and TCFH (211.3 mg, 752.9 μmol) at room temperature. The resulting mixture was stirred for 16 h at room temperature, diluted with water (5 mL), and then extracted with EtOAc (3×5 mL). The combined organic layers were washed with water (2×15 mL), brine (15 mL), dried over anhydrous Na₂SO₄, and filtered. The filtrate was concentrated under reduced pressure and purified by silica gel column chromatography (9% MeOH in DCM) to afford tert-butyl N-[(3S)-1-[7-[(8-fluoro-2-methyl-imidazo[1,2-a]pyridin-6-yl)carbamoyl]-2-methyl-indazol-4-yl]pyrrolidin-3-yl]-N-prop-2-ynyl-carbamate (A47, 100 mg, 183.3 μmol). LCMS (ES, m/z): 546 [M+H]⁺.

### Synthesis of Compound 121

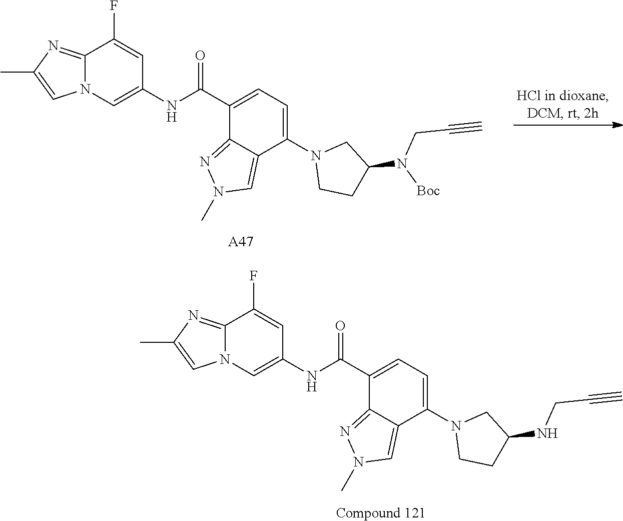

To a stirred mixture of Intermediate A47 (100 mg, 183.3 μmol) in DCM (2 mL) was added HCl (4 M in dioxane) (0.4 mL) dropwise, and the reaction was stirred for 2 h at room temperature. The resulting mixture was concentrated under reduced pressure, diluted with water (5 mL), basified to pH 8 with saturated NaHCO₃ (aq.), and extracted with DCM (3×5 mL). The combined organic layers were washed with water (2×10 mL), brine (10 mL), dried over anhydrous Na₂SO₄, and filtered. The filtrate was concentrated under reduced pressure and purified by Prep-HPLC (Condition 5, Gradient 1) to afford N-(8-fluoro-2-methyl-imidazo[1,2-a]pyridin-6-yl)-2-methyl-4-[(3S)-3-(prop-2-ynylamino)pyrrolidin-1-yl]indazole-7-carboxamide (Compound 121, 40 mg, 89.8 μmol). LCMS (ES, m/z): 446 [M+H]⁺. ¹H NMR (400 MHz, DMSO-d₆) δ 11.01 (s, 1H), 9.20 (d, J=1.6 Hz, 1H), 8.83 (s, 1H), 7.98-7.84 (m, 2H), 7.31 (dd, J=12.4, 1.7 Hz, 1H), 6.01 (d, J=8.4 Hz, 1H), 4.27 (s, 3H), 3.86-3.61 (m, 4H), 3.52-3.39 (m, 3H), 3.12 (t, J=2.4 Hz, 1H), 2.51-2.50 (m, 1H), 2.35 (s, 3H), 2.15 (dq, J=13.0, 7.2 Hz, 1H), 1.96 (dd, J=11.5, 6.1 Hz, 1H), 1.06 (t, J=7.0 Hz, 1H).

### Example 12: Synthesis of Compound 124

### Synthesis of Intermediate A48

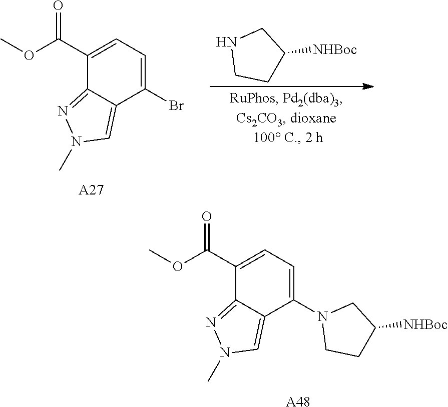

To a stirred mixture of Intermediate A27 (300 mg, 1.11 mmol) and tert-butyl N-[(3R)-pyrrolidin-3-yl]carbamate (249.2 mg, 1.34 mmol) in dioxane (6 mL) were added Cs₂CO₃ (363.2 mg, 1.11 mmol), RuPhos (104.1 mg, 223 μmol) and Pd₂(dba)₃ (102.1 mg, 111.5 μmol), and the reaction was stirred for 2 h at 100° C. under N₂. The resulting mixture was allowed to cool to room temperature, diluted with water (10 mL), and extracted with EtOAc (3×10 mL). The combined organic layers were washed with water (3×30 mL), brine (30 mL), dried over anhydrous Na₂SO₄, and filtered. The filtrate was concentrated under reduced pressure and purified by silica gel column chromatography (9% MeOH in DCM) to afford methyl 4-[(3R)-3-(tert-butoxycarbonylamino)pyrrolidin-1-yl]-2-methyl-indazole-7-carboxylate (A48, 450 mg, 1.08 mmol). LCMS (ES, m/z): 375 [M+H]⁺.

### Synthesis of Intermediate A49

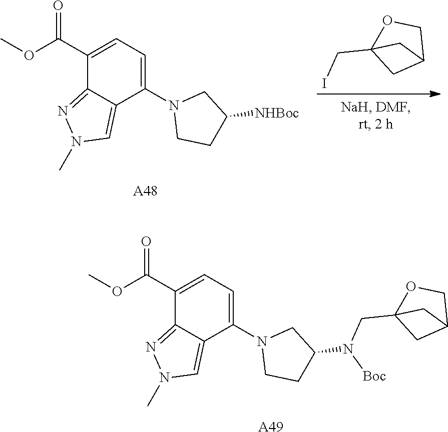 [SMILES: COC(=O)c1ccc(N2CC[C@@H](NC(=O)OC(C)(C)C)C2)c2cn(C)nc12 ; ICC12CC(CO1)C2 ; "A48" ; COC(=O)c1ccc(N2CC[C@@H](N(CC34CC(CO3)C4)C(=O)OC(C)(C)C)C2)c2cn(C)nc12 ; "A49"]

To a solution of Intermediate A48 (450 mg, 1.20 mmol) in DMF (9 mL) was added NaH (60% dispersion in oil; 72.1 mg, 3 mmol) at 0° C. The mixture was stirred for 15 min, then 1-(iodomethyl)-2-oxabicyclo[2.1.1]hexane (350 mg, 1.56 mmol) was added and the mixture was allowed to warm to room temperature and stirred for an additional 2 h. The reaction mixture was quenched by water and extracted with EtOAc (3×10 mL). The combined organic layers were washed with water (3×30 mL), brine (30 mL), dried over anhydrous Na₂SO₄, and filtered. The filtrate was concentrated under reduced pressure and purified by silica gel column chromatography (9% MeOH in DCM) to afford methyl 4-[(3R)-3-[tert-butoxycarbonyl(2-oxabicyclo[2.1.1]hexan-1-ylmethyl)amino]pyrrolidin-1-yl]-2-methyl-indazole-7-carboxylate (A49, 180 mg, 382.5 μmol). LCMS (ES, m/z): 471 [M+H]⁺.

### Synthesis of Intermediate A50

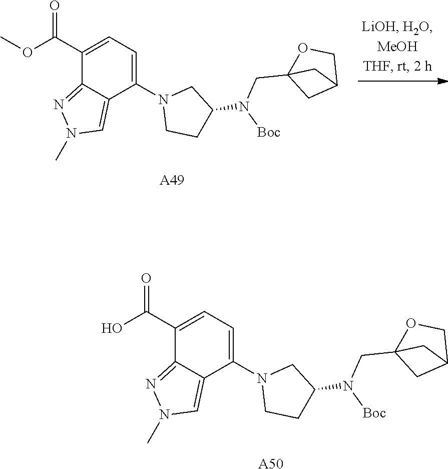 [SMILES: COC(=O)c1ccc(N2CC[C@@H](N(CC34CC(CO3)C4)C(=O)OC(C)(C)C)C2)c2cn(C)nc12 ; "A49" ; Cn1cc2c(N3CC[C@@H](N(CC45CC(CO4)C5)C(=O)OC(C)(C)C)C3)ccc(C(=O)O)c2n1 ; "A50"]

To a stirred mixture of Intermediate A49 (180 mg, 382.5 μmol) in THF (2 mL) were added MeOH (2 mL), H₂O (2 mL) and LiOH·H₂O (80.3 mg, 1.91 mmol), and the reaction was stirred for 2 h at room temperature. The resulting mixture was acidified to pH 5 with HCl (2M) and extracted with EtOAc (3×10 mL). The combined organic layers were washed with water (3×30 mL) and brine (30 mL), dried over anhydrous Na₂SO₄, filtered, and concentrated under reduced pressure to afford 4-[(3R)-3-[tert-butoxycarbonyl(2-oxabicyclo[2.1.1]hexan-1-ylmethyl)amino]-pyrrolidin-1-yl]-2-methyl-indazole-7-carboxylic acid (A50, 90 mg, 197.1 μmol). LCMS (ES, m/z): 457 [M+H]⁺.

### Synthesis of Intermediate A51

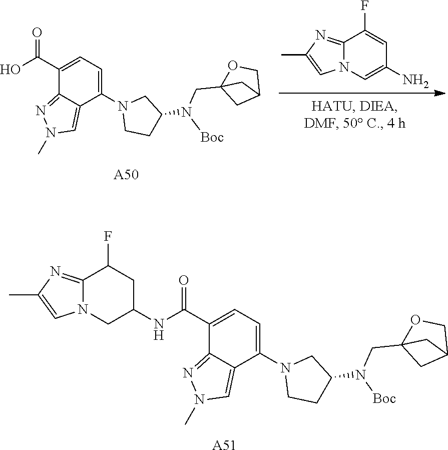

To a stirred mixture of Intermediate A50 (90 mg, 197.1 μmol) and 8-fluoro-2-methyl-imidazo[1,2-a]pyridin-6-amine (39.1 mg, 236.6 μmol) in DMF (2.5 mL) were added DIEA (101.9 mg, 788.6 μmol, 137.4 μL) and HATU (112.4 mg, 295.7 μmol) at room temperature. The resulting mixture was stirred for 4 h at 50° C., diluted with water (10 mL), and extracted with EtOAc (3×10 mL). The combined organic layers were washed with water (3×20 mL) and brine (20 mL), dried over anhydrous Na₂SO₄, and filtered. The filtrate was concentrated under reduced pressure and purified by silica gel column chromatography (9% MeOH in DCM) to afford tert-butyl N-[(3R)-1-[7-[(8-fluoro-2-methyl-imidazo[1,2-a]pyridin-6-yl)-carbamoyl]-2-methyl-indazol-4-yl]pyrrolidin-3-yl]-N-(2-oxabicyclo[2.1.1]hexan-1-ylmethyl)-carbamate (A51, 70 mg, 116 μmol). LCMS (ES, m/z): 604 [M+H]⁺.

### Synthesis of Compound 124

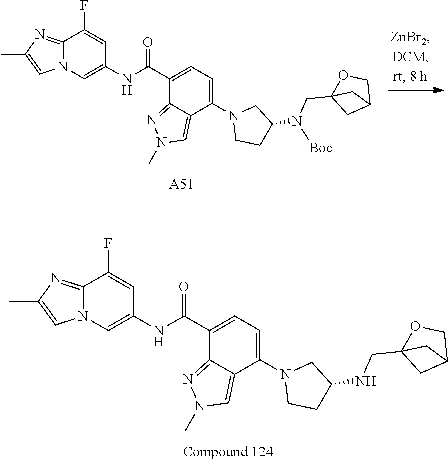

To a stirred mixture of Intermediate A51 (60 mg, 99.39 μmol) in DCM (1.5 mL) was added ZnBr₂ (223.8 mg, 993.9 μmol) at room temperature. The resulting mixture was stirred for 8 h, concentrated under reduced pressure, diluted with water (5 mL), then basified to pH 8 with saturated NaHCO₃ (aq.), and lastly extracted with DCM (3×5 mL). The combined organic layers were washed with water (2×10 mL) and brine (10 mL), dried over anhydrous Na₂SO₄, and filtered. The filtrate was concentrated under reduced pressure and purified by Prep-HPLC (Condition 5, Gradient 2) to afford N-(8-fluoro-2-methyl-imidazo[1,2-a]pyridin-6-yl)-2-methyl-4-[(3R)-3-(2-oxabicyclo[2.1.1]hexan-1-ylmethylamino)pyrrolidin-1-yl]indazole-7-carboxamide (Compound 124, 8 mg, 15.89 μmol). LCMS (ES, m/z): 504 [M+H]⁺. ¹H NMR (400 MHz, CDCl₃) δ 10.96 (s, 1H), 9.23 (s, 1H), 8.19 (d, J=8.3 Hz, 1H), 8.11 (s, 1H), 7.42 (d, J=3.0 Hz, 1H), 6.82 (d, J=11.4 Hz, 1H), 6.06 (d, J=8.4 Hz, 1H), 4.26 (s, 3H), 3.93 (dd, J=9.8, 6.2 Hz, 1H), 3.82-3.81 (m, 3H), 3.68 (q, J=8.4, 8.0 Hz, 2H), 3.59-3.58 (m, 1H), 3.11 (q, J=12.6 Hz, 2H), 2.96 (t, J=3.2 Hz, 1H), 2.50 (s, 3H), 2.39-2.29 (m, 1H), 2.10-2.09 (m, 1H), 1.79-1.78 (m, 2H), 1.58 (d, J=5.1 Hz, 2H).

An analogous method was followed to obtain the following compounds.

| Compound | Starting Material | Characterization |
|---|---|---|
| 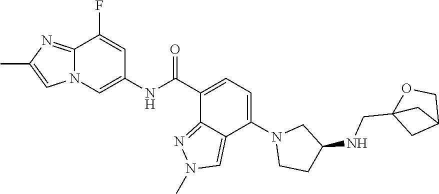 [SMILES: Cc1cn2cc(NC(=O)c3ccc(N4CC[C@H](NCC56CC(CO5)C6)C4)c4cn(C)nc34)cc(F)c2n1] Compound 125 | 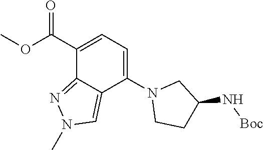 A44 | The crude product was purified by Prep-HPLC (Condition 5, Gradient 2) to afford N-(8- fluoro-2-methyl-imidazo[1,2- a]-pyridin-6-yl)-2-methyl-4- [(3R)-3-(2-oxabicyclo- [2.1.1]hexan-1-ylmethyl- amino)pyrrolidin-1-yl][6.1]- indazole-7-carboxamide (Compound 125, 8 mg, 15.89 μmol). LCMS (ES, m/z): 504 [M + H]⁺. ¹H NMR (400 MHz, CDCl₃) δ 10.95 (s, 1H), 9.23 (d, J = 1.6 Hz, 1H), 8.19 (d, J = 8.3 Hz, 1H), 8.10 (s, 1H), 7.43-7.39 (m, 1H), 6.80 (d, J = 11.3 Hz, |
| | | 1H), 6.05 (d, J = 8.4 Hz, 1H), |
| | | 4.26 (s, 3H), 3.91 (dd, J = 9.7, |
| | | 6.2 Hz, 1H), 3.82 (s, 3H), 3.67 |
| | | (q, J = 9.2, 7.8 Hz, 2H), 3.54 |
| | | (dd, J = 9.8, 5.3 Hz, 1H), 3.10 |
| | | (q, J = 12.5 Hz, 2H), 2.96 (t, J = |
| | | 3.2 Hz, 1H), 2.50-2.49 (m, 3H), |
| | | 2.33 (dq, J = 12.8, 6.4 Hz, 1H), |
| | | 2.06 (dd, J = 12.8, 6.5 Hz, 1H), |
| | | 1.78 (d, J = 3.5 Hz, 2H), 1.60- |
| | | 1.55 (m, 2H). |

### Example 13: Synthesis of Compound 127

### Synthesis of Intermediate A52

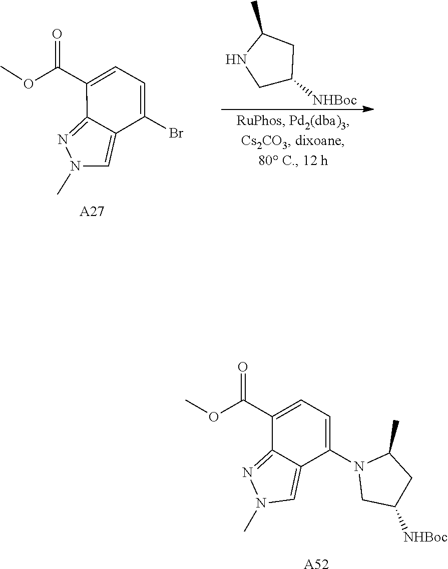

To a solution of tert-butyl N-[(3S,5S)-5-methylpyrrolidin-3-yl]carbamate (164 mg, 817.6 μmol) and methyl Intermediate A27 (0.2 g, 743.2 μmol) in dioxane (4 mL) were added Pd₂(dba)₃ (68 mg, 74.32 μmol), Ruphos (69 mg, 148.7 μmol) and Cs₂CO₃ (726 mg, 2.23 mmol), and the reaction was stirred for 12 h at 80° C. under N₂. The resulting mixture was concentrated under reduced pressure and purified by reversed-phase flash chromatography (Condition 3, Gradient 5) to afford methyl 4-[(2S,4S)-4-(tert-butoxycarbonylamino)-2-methyl-pyrrolidin-1-yl]-2-methyl-indazole-7-carboxylate (A52, 0.23 g, 592.1 μmol). LCMS (ES, m/z): 389 [M+H]⁺.

### Synthesis of Intermediate A53

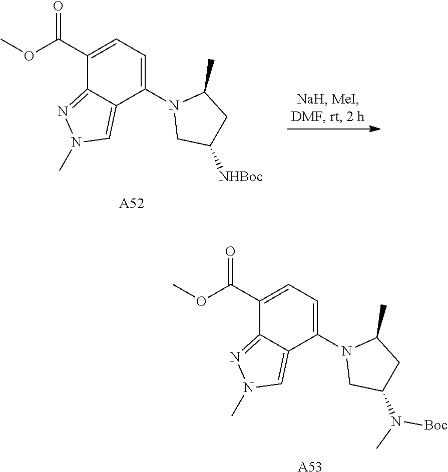 [SMILES: COC(=O)c1ccc(N2C[C@@H](NC(=O)OC(C)(C)C)C[C@@H]2C)c2cn(C)nc12 ; "A52" ; COC(=O)c1ccc(N2C[C@@H](N(C)C(=O)OC(C)(C)C)C[C@@H]2C)c2cn(C)nc12 ; "A53"]

To a solution of Intermediate A52 (0.2 g, 514.9 μmol) in DMF (4 mL) was added NaH (49 mg, 2.06 mmol, 60%) at 0° C. and stirred for 15 min. To above solution was added CH₃I (731 mg, 5.15 mmol), allowed to stir for 2 h at room temperature, diluted with EtOAc (20 mL), quenched with saturated aqueous NH₄Cl (20 mL), and then extracted with EtOAc (3×20 mL). The combined organics were washed with brine (10 mL), dried over anhydrous Na₂SO₄, and filtered. The filtrate was concentrated under reduced pressure and purified by reversed-phase flash chromatography (Condition 3, Gradient 6) to afford methyl 4-[(2S,4S)-4-[tert-butoxycarbonyl(methyl)amino]-2-methyl-pyrrolidin-1-yl]-2-methyl-indazole-7-carboxylate (A53, 0.2 g, 472.1 μmol). LCMS (ES, m/z): 403 [M+H]⁺.

### Synthesis of Intermediate A54

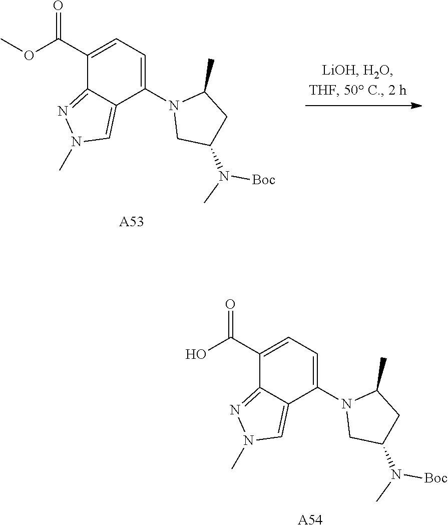 [SMILES: COC(=O)c1ccc(N2C[C@@H](N(C)C(=O)OC(C)(C)C)C[C@@H]2C)c2cn(C)nc12 ; "A53" ; C[C@H]1C[C@H](N(C)C(=O)OC(C)(C)C)CN1c1ccc(C(=O)O)c2nn(C)cc12 ; "A54"]

To a stirred solution of Intermediate A53 (0.2 g, 496.9 μmol) in MeOH (1 mL) and THE (1 mL) was added a solution of LiOH·H₂O (209 mg, 4.97 mmol) in H₂O (1 mL) at room temperature. The resulting mixture was stirred for 2 h at 50° C., acidified to pH 4 with 0.5 M aq. HCl, and then extracted with DCM/MeOH (3×5 mL). The combined organic layers were washed with brine (2×10 mL), dried over anhydrous Na₂SO₄, and filtered. The filtrate was concentrated under reduced pressure and purified by reversed-phase flash chromatography (Condition 3, Gradient 7) to afford 4-[(2S,4S)-4-[tert-butoxycarbonyl(methyl)amino]-2-methyl-pyrrolidin-1-yl]-2-methyl-indazole-7-carboxylic acid (A54, 0.14 g, 360.4 μmol). LCMS (ES, m/z): 389 [M+H]⁺.

### Synthesis of Intermediate A55

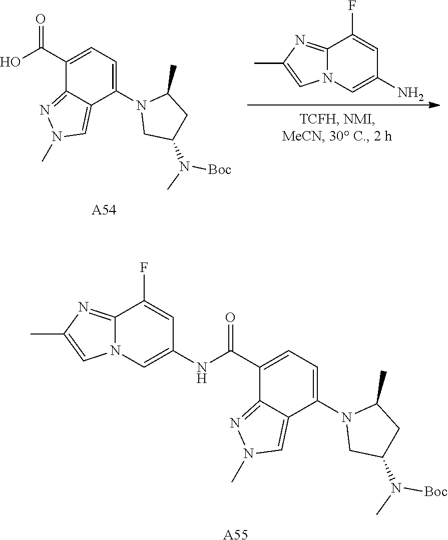 [SMILES: C[C@H]1C[C@H](N(C)C(=O)OC(C)(C)C)CN1c1ccc(C(=O)O)c2nn(C)cc12 ; Cc1cn2cc(N)cc(F)c2n1 ; "A54" ; Cc1cn2cc(NC(=O)c3ccc(N4C[C@@H](N(C)C(=O)OC(C)(C)C)C[C@@H]4C)c4cn(C)nc34)cc(F)c2n1 ; "A55"]

To a stirred solution of Intermediate A54 (0.1 g, 257.4 μmol), TCFH (0.14 g, 515 μmol) and NMI (85 mg, 1.03 mmol) in MeCN (2 mL) was added 8-fluoro-2-methyl-imidazo[1,2-a]-pyridin-6-amine (64 mg, 386.1 μmol), and the reaction was stirred for 2 h at 30° C. under N₂. The resulting mixture was concentrated under reduced pressure and purified by reversed-phase flash chromatography (Condition 3, Gradient 6) to afford tert-butyl N-[(3S,5S)-1-[7-[(8-fluoro-2-methyl-imidazo[1,2-a]pyridin-6-yl)carbamoyl]-2-methyl-indazol-4-yl]-5-methyl-pyrrolidin-3-yl]-N-methyl-carbamate (A55, 0.05 g, 93.35 μmol). LCMS (ES, m/z): 536 [M+H]⁺.

### Synthesis of Compound 127

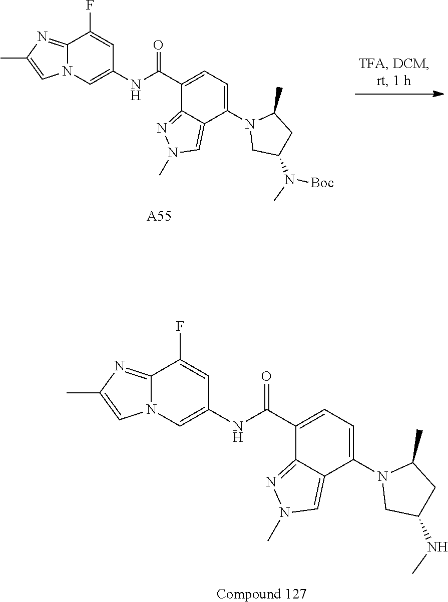 [SMILES: Cc1cn2cc(NC(=O)c3ccc(N4C[C@@H](N(C)C(=O)OC(C)(C)C)C[C@@H]4C)c4cn(C)nc34)cc(F)c2n1 ; "A55" ; CN[C@H]1C[C@H](C)N(c2ccc(C(=O)Nc3cc(F)c4nc(C)cn4c3)c3nn(C)cc23)C1 ; "Compound 127"]

To a stirred solution of Intermediate A55 (0.06 g, 112.0 μmol) in DCM (1 mL) was added TFA (0.1 mL), and the reaction was stirred for 1 h at room temperature. The resulting mixture was basified to pH 8 with NH₃ (g) in MeOH, concentrated under reduced pressure, and purified by reversed-phase flash chromatography (Condition 3, Gradient 4) to afford N-(8-fluoro-2-methyl-imidazo[1,2-a]pyridin-6-yl)-2-methyl-4-[(2S,4S)-2-methyl-4-(methylamino)-pyrrolidin-1-yl]indazole-7-carboxamide (Compound 127, 17 mg, 39.04 μmol). LCMS (ES, m/z): 436 [M+H]⁺. ¹H NMR (300 MHz, DMSO-d₆) δ 11.03 (s, 1H), 9.20 (d, J=1.6 Hz, 1H), 8.78 (s, 1H), 7.97-7.85 (m, 2H), 7.31 (dd, J=12.6, 1.6 Hz, 1H), 6.08 (d, J=8.5 Hz, 1H), 4.33 (d, J=5.8 Hz, 1H), 4.28 (s, 3H), 4.00 (dd, J=9.9, 6.3 Hz, 1H), 3.54-3.43 (m, 1H), 3.43-3.32 (m, 1H), 2.35 (d, J=1.7 Hz, 6H), 1.96 (t, J=6.1 Hz, 6H), 1.25 (d, J=6.2 Hz, 3H).

### Example 14: Synthesis of Compound 132

### Synthesis of Intermediate A59

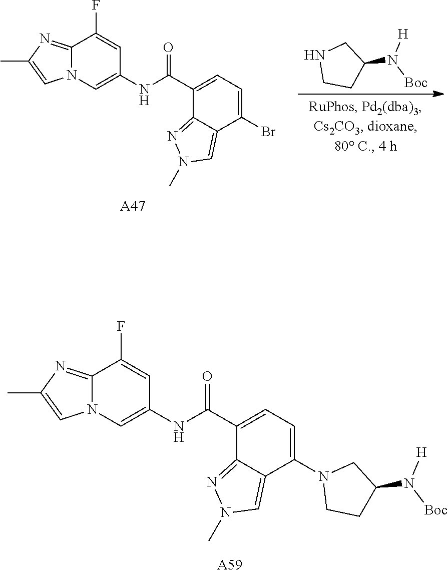

To a stirred mixture of Intermediate A7 (220 mg, 0.55 mmol, 1 eq) and tert-butyl N-[(3S)-pyrrolidin-3-yl]carbamate (122 mg, 0.66 mmol, 1.2 eq) in dioxane (5 mL) were added Cs₂CO₃ (356 mg, 1.1 mmol, 2 eq), RuPhos (51 mg, 0.11 mmol, 0.2 eq) and Pd₂(dba)₃ (50 mg, 0.055 mmol, 0.1 eq) in portions, and the reaction was stirred for 4 h at 80° C. under N₂. The resulting mixture was concentrated under reduced pressure and purified by silica gel column chromatography (67% EtOAc in PE) to afford tert-butyl N-[(3S)-1-[7-((8-fluoro-2-methylimidazo[1,2-a]pyridin-6-yl)carbamoyl)-2-methylindazol-4-yl]pyrrolidin-3-yl]carbamate (A59, 240 mg). LCMS (ES, m/z): 508 [M+H]⁺.

### Synthesis of Intermediate A60

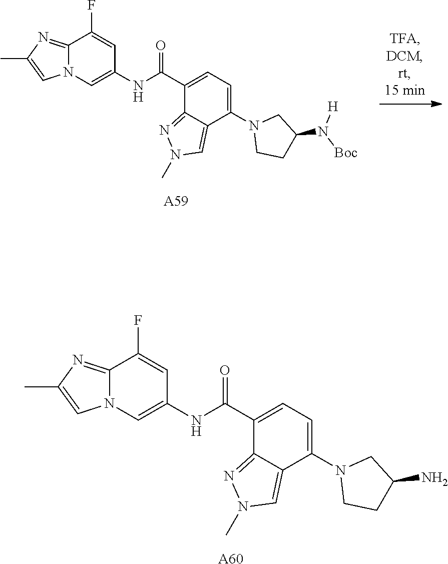

A solution of Intermediate A59 (240 mg, 0.47 mmol, 1 eq) and TFA (0.5 mL) in DCM (1.5 mL) was stirred for 15 min at room temperature. The resulting mixture was concentrated under reduced pressure, basified to pH 8 with saturated NaHCO₃ (aq.), and extracted with DCM (4×10 mL). The combined organic layers were washed with brine (2×5 mL), dried over anhydrous Na₂SO₄, and filtered. The filtrate was concentrated under reduced pressure and purified by silica gel column chromatography (9% MeOH in DCM) to afford 4-[(3S)-3-aminopyrrolidin-1-yl]-N-(8-fluoro-2-methylimidazo[1,2-a]pyridin-6-yl)-2-methyl-indazole-7-carboxamide (A60, 160 mg). LCMS (ES, m/z): 408 [M+H]⁺.

### Synthesis of Compound 132

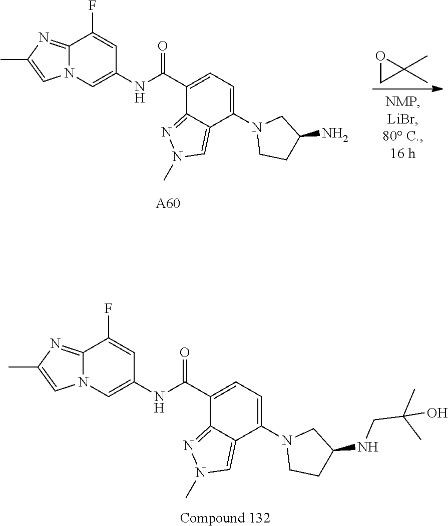

To a stirred mixture of Intermediate A60 (100 mg, 245.4 μmol) in NMP (2 mL) were added 2,2-dimethyloxirane (46 mg, 638.1 μmol) and LiBr (85 mg, 981.7 μmol) at room temperature. The resulting mixture was stirred for 16 h at 80° C., quenched with water (20 mL), and then extracted with DCM (4×20 mL). The combined organic layers were washed with brine (2×10 mL), dried over anhydrous Na₂SO₄, and filtered. The filtrate was concentrated under reduced pressure and purified by reverse flash chromatography (Condition 3, Gradient 3) to afford N-(8-fluoro-2-methyl-imidazo[1,2-a]pyridin-6-yl)-4-[(3S)-3-[(2-hydroxy-2-methylpropyl)amino]-pyrrolidin-1-yl]-2-methyl-indazole-7-carboxamide (Compound 132, 12 mg, 25.02 μmol). LCMS (ES, m/z): 480 [M+H]⁺. ¹H NMR (400 MHz, DMSO-d₆) δ 11.01 (s, 1H), 9.19 (d, J=1.6 Hz, 1H), 8.84 (s, 1H), 7.93 (d, J=8.2 Hz, 1H), 7.88 (d, J=3.0 Hz, 1H), 7.31 (d, J=12.4 Hz, 1H), 6.03 (d, J=8.4 Hz, 1H), 4.27 (s, 3H), 4.18 (s, 1H), 3.79 (d, J=30.3 Hz, 2H), 3.65 (s, 1H), 3.46 (s, 2H), 3.28 (s, 1H), 2.35 (s, 3H), 2.18 (d, J=5.8 Hz, 1H), 1.92 (d, J=6.5 Hz, 1H), 1.11 (s, 6H).

### Example 15: Synthesis of Compound 135

### Synthesis of Intermediate A62

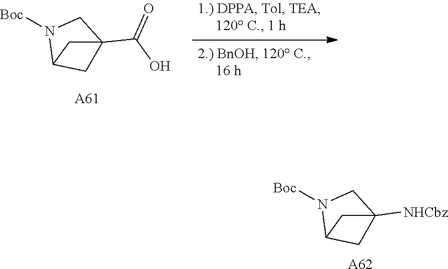

To a stirred mixture of 2-tert-butoxycarbonyl-2-azabicyclo[2.1.1]hexane-4-carboxylic acid (A61, 370 mg, 1.63 mmol) and TEA (345.9 mg, 3.42 mmol, 476.6 μL) in toluene (7 mL) was added DPPA (707.9 mg, 2.57 mmol, 554.4 μL) at room temperature. The resulting mixture was stirred for 1 h at 120° C. under N₂ and allowed to cool to room temperature. BnOH (369.7 mg, 3.42 mmol, 353.8 μL) was then added, the resulting mixture was stirred for an additional 16 h at 120° C., cooled to room temperature, diluted with water (7 mL), and extracted with EtOAc (2×7 mL). The combined organic layers were washed with water (2×15 mL), dried over anhydrous Na₂SO₄, and filtered. The filtrate was concentrated under reduced pressure and purified by silica gel column chromatography (25% EtOAc in PE) to afford tert-butyl 4-(benzyloxycarbonylamino)-2-azabicyclo[2.1.1]hexane-2-carboxylate (A62, 400 mg, 1.20 mmol). LCMS (ES, m/z): 396 [M+Na+CH₃CN]⁺.

### Synthesis of Intermediate A63

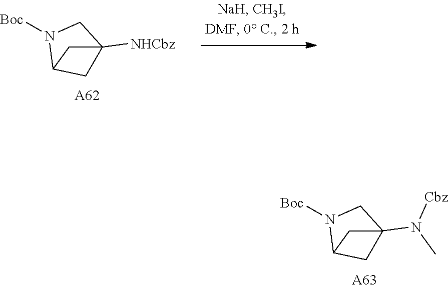

To a stirred mixture of Intermediate A62 (210 mg, 631.8 μmol) in DMF (4 mL) was added NaH (60% dispersion in oil; 45.5 mg, 1.9 mmol) at 0° C. The resulting mixture was stirred for 0.5 h at room temperature under N₂. Following addition of iodomethane (269 mg, 1.9 mmol, 118 μL), the mixture was stirred for additional 2 h, quenched by ice water (15 mL), and then extracted with EtOAc (3×20 mL). The combined organic layers were washed with water (2×50 mL), brine (50 mL), dried over anhydrous Na₂SO₄, and filtered. The filtrate was concentrated under reduced pressure and purified by silica gel column chromatography (25% EtOAc in PE) to afford tert-butyl 4-[benzyloxycarbonyl(methyl)amino]-2-azabicyclo[2.1.1]hexane-2-carboxylate (A63, 200 mg, 577.3 μmol). LCMS (ES, m/z): 347 [M+H]⁺.

### Synthesis of Intermediate A64

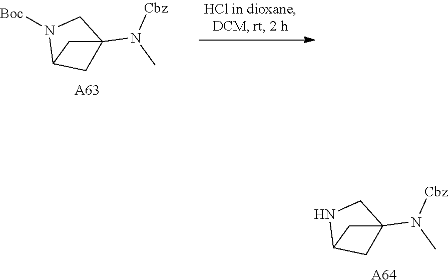

To a stirred mixture of Intermediate A63 (220 mg, 635.1 μmol) in DCM (4 mL) was added HCl (4.0 M in 1,4-dioxane, 2 mL), and the reaction was stirred for 2 h at room temperature under N₂. The resulting mixture was concentrated under reduced pressure and washed with MTBE (2 mL) to yield benzyl(2-azabicyclo[2.1.1]hexan-4-yl)(methyl)carbamate hydrochloride (A64, 160 mg, 565.8 μmol). LCMS (ES, m/z): 247 [M+H]⁺.

### Synthesis of Intermediate A65

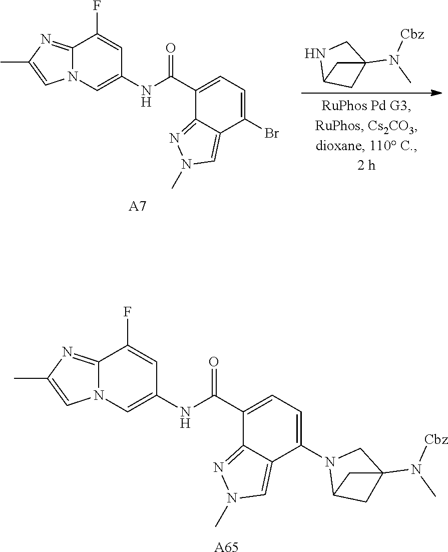

To a stirred mixture of Intermediate A7 (135 mg, 335.6 μmol) and Intermediate A64 (94.3 mg, 335.6 μmol) in dioxane (0.5 mL) were added Cs₂CO₃ (327.2 mg, 1.01 mmol), Ruphos (31.9 mg, 67.1 μmol) and Pd₂(dba)₃ (30.7 mg, 33.56 μmol), and the reaction was stirred for 2 h at 110° C. under N₂. The resulting mixture was concentrated under reduced pressure and purified by silica gel column chromatography (67% EtOAc in PE) to afford benzyl(2-(7-((8-fluoro-2-methylimidazo[1,2-a]pyridin-6-yl)carbamoyl)-2-methyl-2H-indazol-4-yl)-2-azabicyclo[2.1.1]-hexan-4-yl)(methyl)carbamate (A65, 110 mg, 194 μmol). LCMS (ES, m/z): 568 [M+H]⁺.

### Synthesis of Compound 135

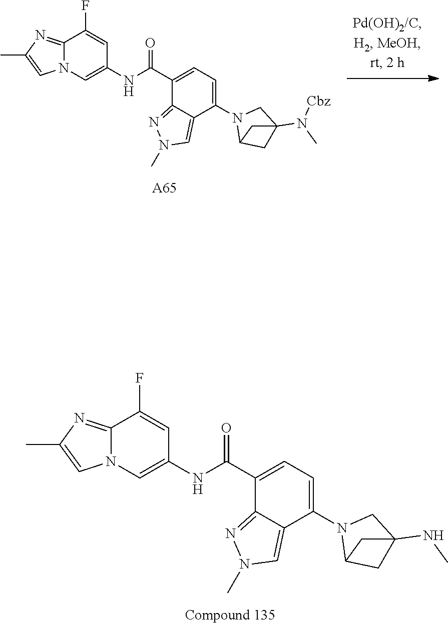

To a solution of Intermediate A65 (110 mg, 193.8 μmol) in MeOH (2.2 mL) was added Pd(OH)₂ on carbon (27 mg) under N₂ in a 40 mL round-bottom flask. The mixture was hydrogenated at room temperature for 2 h under hydrogen atmosphere using a hydrogen balloon. The resulting mixture was filtered through a Celite pad, concentrated under reduced pressure, and purified by Prep-HPLC (Condition 5, Gradient 2) to afford N-(8-fluoro-2-methyl-imidazo[1,2-a]pyridin-6-yl)-2-methyl-4-[4-(methylamino)-2-azabicyclo[2.1.1]hexan-2-yl]-indazole-7-carboxamide (Compound 135, 30 mg, 69.21 μmol). LCMS (ES, m/z): 434 [M+H]⁺. ¹H NMR (400 MHz, DMSO-d₆) δ 10.97 (s, 1H), 9.19 (d, J=1.6 Hz, 1H), 8.80 (s, 1H), 7.95-7.82 (m, 2H), 7.31 (dd, J=12.4, 1.7 Hz, 1H), 6.31 (d, J=8.3 Hz, 1H), 4.57 (s, 1H), 4.28 (s, 3H), 3.54 (s, 2H), 2.39 (s, 3H), 2.35 (s, 3H), 1.98-1.92 (m, 2H), 1.56 (dd, J=4.3, 1.8 Hz, 2H).

An analogous method was followed to obtain the following compounds.

| Compound | Starting Material | Characterization |
|---|---|---|
| 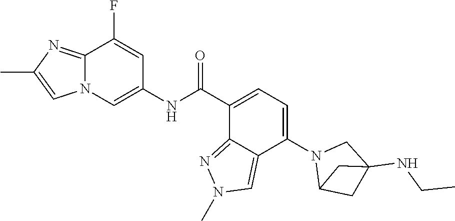 [SMILES: CCNC12CC(C1)N(c1ccc(C(=O)Nc3cc(F)c4nc(C)cn4c3)c3nn(C)cc13)C2] Compound 136 | EtI was used in Step 1 | The crude product was purified by Prep- HPLC (Condition 5, Gradient 1) to afford 4-[4-(ethylamino)-2-azabicyclo- [2.1.1]hexan-2-yl]N-(8-fluoro-2- methylimidazo[1,2-a]pyridin-6-yl)-2- methyl-indazole-7-carboxamide (Compound 136, 20 mg, 44.69 μmol). LCMS (ES, m/z): 448 [M + H]⁺. ¹H NMR (400 MHz, DMSO-d₆) δ 10.97 (s, 1H), 9.19 (d, J = 1.6 Hz, 1H), 8.79 (s, 1H), 7.94-7.87 (m, 2H), 7.31 (dd, J = 12.4, 1.7 Hz, 1H), 6.30 (d, J = 8.3 Hz, 1H), 4.56 (s, 1H), 4.28 (s, 3H), 3.54 (s, 2H), 2.69 (q, J = 7.1 Hz, 2H), 2.35 (s, 3H), 1.95 (s, 2H), 1.58 (dd, J = 4.3, 1.8 Hz, 2H), 1.07 (t, J = 7.1 Hz, 3H). |

### Example 16: Synthesis of Compound 137

### Synthesis of Intermediate A70

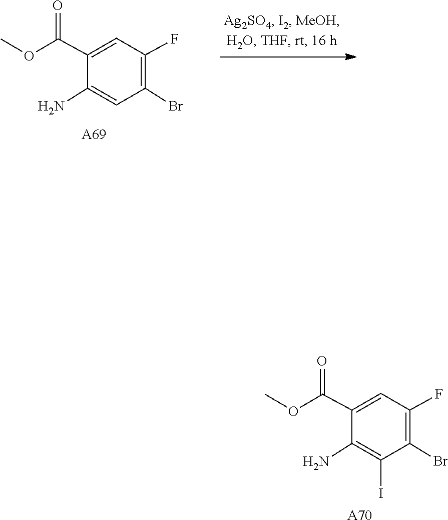

To a stirred mixture of methyl 2-amino-4-bromo-5-fluoro-benzoate (A69, 25 g, 100.8 mmol) and Ag₂SO₄ (50.3 g, 161.3 mmol) in MeOH (15 mL) was added I2 (25.8 g, 161.3 mmol) in portions at room temperature. The resulting mixture was stirred for 16 h, with monitoring by LCMS, then diluted with water (500 ml) and extracted with EtOAc (500 mL). The combined organic layers were washed with brine (500 mL), dried over anhydrous Na₂SO₄, and filtered. The filtrate was concentrated under reduced pressure and purified by silica gel column chromatography (17% EtOAc in PE) to afford methyl 2-amino-4-bromo-5-fluoro-3-iodo-benzoate (A70, 30 g, 80.2 mmol). LCMS (ES, m/z): 374 [M+H]⁺.

### Synthesis of Intermediate A71

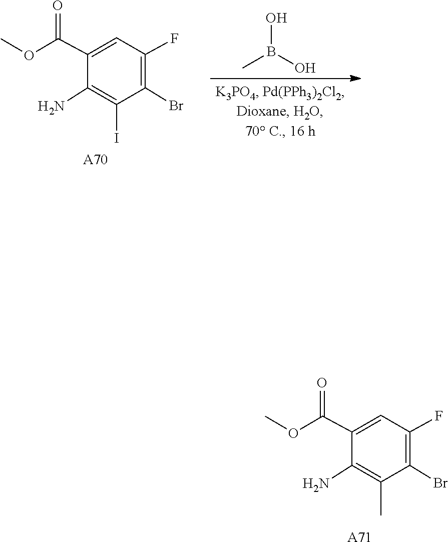

To a solution of Intermediate A70 (30 g, 82.9 mmol) and methylboronic acid (24.81 g, 414.5 mmol) in dioxane (500 mL) and H₂O (50 mL) were added K₃PO₄ (35.2 g, 165.8 mmol) and Pd(PPh₃)₂Cl₂ (5.8 g, 8.3 mmol), and the reaction was stirred for 16 h at 70° C. under N₂. The resulting mixture was concentrated under reduced pressure and purified by silica gel column chromatography (50% EtOAc in PE) to afford methyl 2-amino-4-bromo-5-fluoro-3-methyl-benzoate (A71, 15 g, 57.2 mmol). LCMS (ES, m/z): 262 [M+H]⁺.

### Synthesis of Intermediate A72

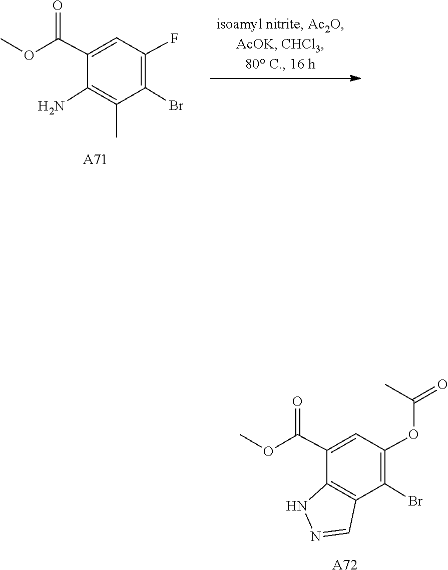

A solution of Intermediate A71 (18 g, 68.7 mmol) in CHCl₃ (300 mL) was treated with Ac₂O (10.5 g, 103 mmol, 9.7 mL) for 1 h at room temperature under N₂ followed by the addition of AcOK (3.37 g, 34.34 mmol, 2.15 mL) and isoamyl nitrite (12 g, 103 mmol), and the reaction was stirred for 16 h at 80° C. The resulting mixture was diluted with water (500 ml) and extracted with DCM (2×500 mL). The combined organic layers were washed with water (500 mL), brine (500 mL), dried over anhydrous Na₂SO₄, and filtered. The filtrate was concentrated under reduced pressure and purified by silica gel column chromatography (50% EtOAc in PE) to afford methyl 5-acetoxy-4-bromo-1H-indazole-7-carboxylate (A72, 13 g, 41.5 mmol). LCMS (ES, m/z): 313 [M+H]⁺.

### Synthesis of Intermediate A73

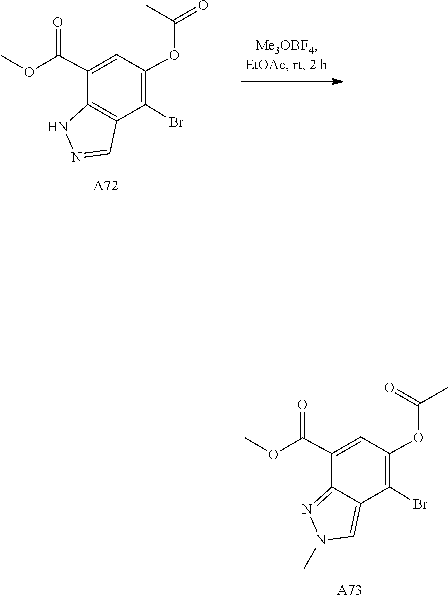

To a stirred mixture of Intermediate A72 (13 g, 41.5 mmol) in EtOAc (260 mL) was added Me₃OBF₄ (7.99 g, 53.98 mmol) at room temperature. The resulting mixture was stirred for 2 h, diluted with NaHCO₃ (aq.) (300 mL), and extracted with EtOAc (2×200 mL). The combined organic layers were washed with brine (200 mL), dried over anhydrous Na₂SO₄, and filtered. The filtrate was concentrated under reduced pressure and purified by silica gel column chromatography (50% EtOAc in PE) to afford methyl 5-acetoxy-4-bromo-2-methyl-indazole-7-carboxylate (A73, 12.5 g, 38.2 mmol). LCMS (ES, m/z): 327 [M+H]⁺.

An analogous method to this step was followed to obtain the following compound.

| Compound | Starting Material | Characterization |
|---|---|---|
| 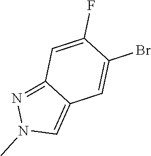 C120 | 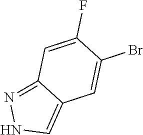 [SMILES: Fc1cc2n[nH]cc2cc1Br] | The residue was purified by silica gel column chromatography (33% EtOAc in PE) to afford 5-bromo-6-fluoro-2-methyl- indazole (C120, 1.49 g, 6.5 mmol, 80% yield) as a solid. LCMS (ES, m/z): 229 [M + H]⁺. |
| 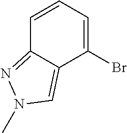 B166 | 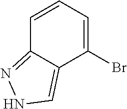 | Modification: The reaction was ran for 1 h. The filtrate was concentrated under reduced pressure to afford 4-bromo-2-methyl-indazole (B166, 4.3 g, 20.4 mmol, 80% yield) as a solid. LCMS (ES, m/z): 211 [M + H]⁺. |
| 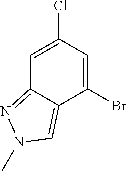 B170 | 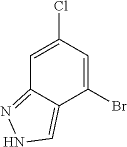 [SMILES: Clc1cc(Br)c2c[nH]nc2c1] | Modification: The reaction was ran for 1 h. The residue was purified by silica gel column chromatography (80% EtOAc in PE) to afford 4-bromo-6-chloro-2-methyl- indazole (B170, 4 g, 16.3 mmol, 75% yield) as a solid. LCMS (ES, m/z): 245 [M + H]⁺. |
| 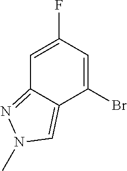 B176 | 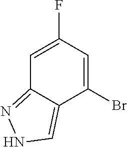 [SMILES: Fc1cc(Br)c2c[nH]nc2c1] | Modification: The reaction was ran for 1 h. The residue was purified by silica gel column chromatography (50% EtOAc in PE) to afford 4-bromo-6-fluoro-2-methyl- indazole (B176, 4 g, 17.5 mmol, 75% yield) as a solid. LCMS (ES, m/z): 229 [M + H]⁺. |
| 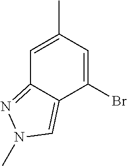 D9 | 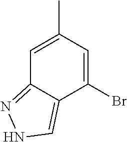 | Modification: The reaction was ran for 1 h. The residue was purified by silica gel column chromatography (25% EtOAc in PE) to afford 4-bromo-2,6-dimethyl-indazole (D9, 4.8 g, 21.3 mmol). LCMS (ES, m/z): 225 [M + H]⁺. |
| 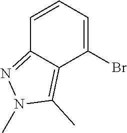 D47 | 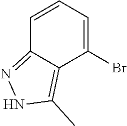 | The residue was purified by silica gel column chromatography (20% EtOAc in PE) to afford 4-bromo-2,3-dimethyl-indazole (D47, 1.39 g, 6.18 mmol). LCMS (ES, m/z): 225 [M + H]⁺. |
| 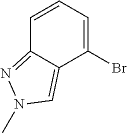 D73 | 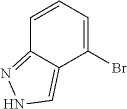 | The residue was purified by silica gel column chromatography (25% EtOAc in PE) to afford 4-bromo-2-methyl-indazole (D73, 15 g, 71.1 mmol). LCMS (ES, m/z): 211 [M + H]⁺. |

### Synthesis of Intermediate A74

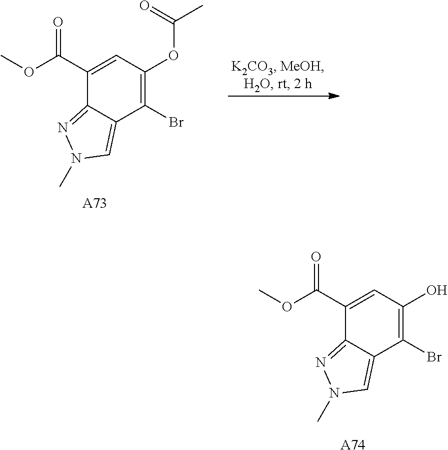

To a stirred mixture of Intermediate A73 (1.6 g, 5.62 mmol) in DMF (30 mL) was added CH₃I (800 mg, 5.62 mmol) dropwise, and the reaction was stirred for 2 h at room temperature. The resulting mixture was quenched with water (40 mL) and extracted with EtOAc (40 mL). The combined organic layers were washed with brine (40 mL), dried over anhydrous Na₂SO₄, and filtered. The filtrate was concentrated under reduced pressure and purified by silica gel column chromatography (33% EtOAc in PE) to afford methyl 4-bromo-5-methoxy-2-methyl-indazole-7-carboxylate (A74, 1.1 g, 3.68 mmol). LCMS (ES, m/z): 285 [M+H]⁺.

### Synthesis of Intermediate A75

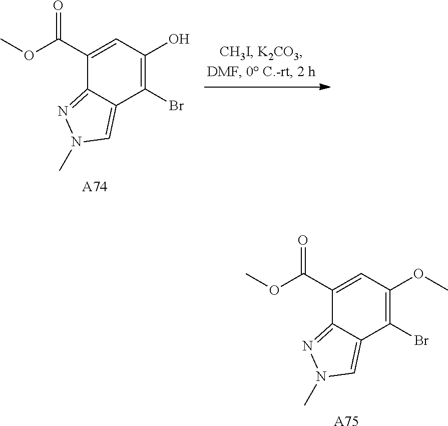

To a stirred mixture of Intermediate A74 (800 mg, 2.81 mmol), K₂CO₃ (581.6 mg, 4.21 mmol) in DMF (15 mL) was added CH₃I (398.4 mg, 2.81 mmol) dropwise at room temperature. The resulting mixture was stirred for 2 h while being monitored by LCMS, then diluted with water (40 mL), and later extracted with EtOAc (2×40 mL). The combined organic layers were washed with water (3×80 mL) brine (80 mL), dried over anhydrous Na₂SO₄, and filtered. The filtrate was concentrated under reduced pressure and purified by silica gel column chromatography (33% EtOAc in PE) to afford methyl 4-bromo-5-methoxy-2-methyl-indazole-7-carboxylate (A75, 550 mg, 1.84 mmol). LCMS (ES, m/z): 299 [M+H]⁺.

### Synthesis of Intermediate A76

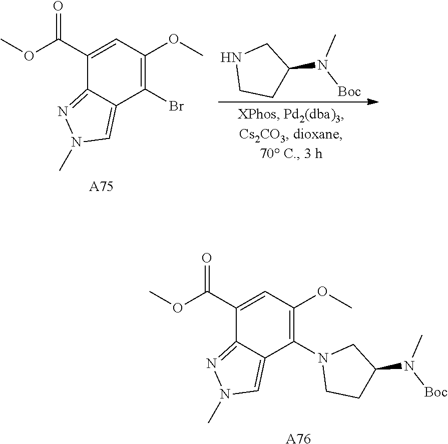

To a solution of Intermediate A75 (540 mg, 1.81 mmol) and tert-butyl N-methyl-N-[(3S)-pyrrolidin-3-yl]carbamate (433.8 mg, 2.17 mmol) in dioxane (10 mL) and were added Cs₂CO₃ (1.18 g, 3.61 mmol), Ruphos (168.4 mg, 361.1 μmol) and Pd₂(dba)₃ (165.8 mg, 181.0 μmol), and the reaction was stirred for 5 h at 80° C. under N₂. The resulting mixture was concentrated under reduced pressure and purified by silica gel column chromatography (50% EtOAc in PE) to afford methyl 4-[(3S)-3-[tert-butoxycarbonyl(methyl)amino]pyrrolidin-1-yl]-5-methoxy-2-methylindazole-7-carboxylate (A76, 700 mg, 1.67 mmol). LCMS (ES, m/z): 419 [M+H]⁺.

### Synthesis of Intermediate A77

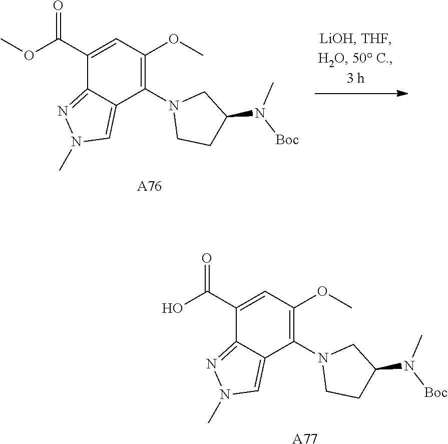 [SMILES: COC(=O)c1cc(OC)c(N2CC[C@H](N(C)C(=O)OC(C)(C)C)C2)c2cn(C)nc12 ; "A76" ; COc1cc(C(=O)O)c2nn(C)cc2c1N1CC[C@H](N(C)C(=O)OC(C)(C)C)C1 ; "A77"]

To a stirred mixture of Intermediate A76 (700 mg, 1.67 mmol) in THF (5 mL) and H₂O (5 mL) was added LiOH (280.4 mg, 11.7 mmol), and the reaction was stirred for 3 h at 50° C. The resulting mixture was diluted with water (50 mL), acidified to pH 5 with HCl (1 N), and extracted with EtOAc (100 mL). The combined organic layers were washed with brine (100 mL), dried over anhydrous Na₂SO₄, filtered, and concentrated under reduced pressure to afford 4-[(3S)-3-[tert-butoxycarbonyl(methyl)amino]pyrrolidin-1-yl]-5-methoxy-2-methyl-indazole-7-carboxylic acid (A77, 500 mg, 1.24 mmol). LCMS (ES, m/z): 405 [M+H]⁺.

### Synthesis of Intermediate A78

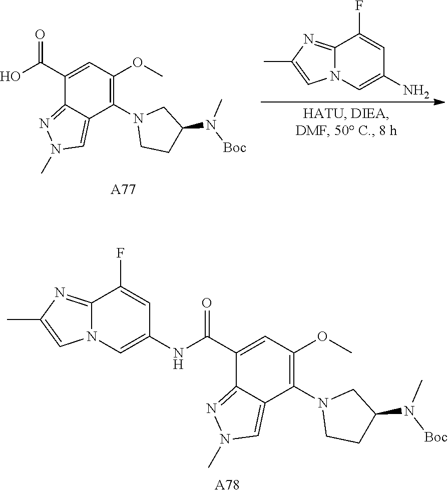

To a stirred mixture of Intermediate A77 (500.0 mg, 1.26 mmol) in DMF (10 mL) were added 8-fluoro-2-methylimidazo[1,2-a]pyridin-6-amine dihydrochloride (448.0 mg, 1.89 mmol), DIEA (812.7 mg, 6.30 mmol), and HATU (958.9 mg, 2.52 mmol) at room temperature. The resulting mixture was stirred for 8 h at 50° C. while being monitored by LCMS, then diluted with water (50 ml), and later extracted with EtOAc (2×50 mL). The combined organic layers were washed with water (2×100 mL), brine (100 mL), dried over anhydrous Na₂SO₄, and filtered. The filtrate was concentrated under reduced pressure and purified by silica gel column chromatography (67% EtOAc in PE) to afford tert-butyl N-[(3S)-1-[7-[(8-fluoro-2-methyl-imidazo[1,2-a]pyridin-6-yl)carbamoyl]-5-methoxy-2-methyl-indazol-4-yl]pyrrolidin-3-yl]-N-methyl-carbamate (A78, 500 mg, 906.4 μmol). LCMS (ES, m/z): 552 [M+H]⁺.

### Synthesis of Compound 137

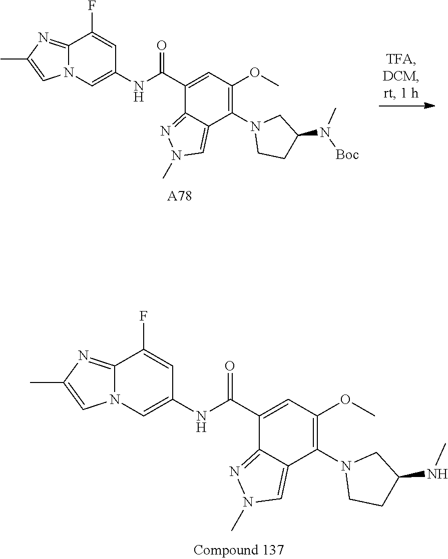

To a stirred solution of Intermediate A78 (130 mg, 235.7 μmol) in DCM (2 mL) was added TFA (0.5 mL) dropwise, and the reaction was stirred for 1 h at room temperature. The resulting mixture was concentrated under reduced pressure and purified by Prep-HPLC (Condition 6, Gradient 1) to afford N-(8-fluoro-2-methyl-imidazo[1,2-a]pyridin-6-yl)-5-methoxy-2-methyl-4-[(3S)-3-(methylamino)pyrrolidin-1-yl]indazole-7-carboxamide (Compound 137, 40 mg, 88.59 μmol). LCMS (ES, m/z): 452 [M+H]⁺. ¹H NMR (400 MHz, DMSO-d₆) δ 11.02 (s, 1H), 9.17 (d, J=1.6 Hz, 1H), 8.84 (s, 1H), 7.89 (d, J=3.1 Hz, 1H), 7.81 (s, 1H), 7.32 (dd, J=12.5, 1.7 Hz, 1H), 4.25 (s, 3H), 4.04-3.91 (m, 2H), 3.85-3.81 (m, 1H), 3.71 (s, 3H), 3.61 (dd, J=10.6, 4.2 Hz, 1H), 3.28-3.27 (m, 1H), 2.35 (d, J=2.8 Hz, 6H), 2.07 (dt, J=12.5, 6.5 Hz, 1H), 1.85 (dd, J=11.9, 6.3 Hz, 1H).

### Example 17: Synthesis of Compound 173

### Synthesis of Intermediate A80

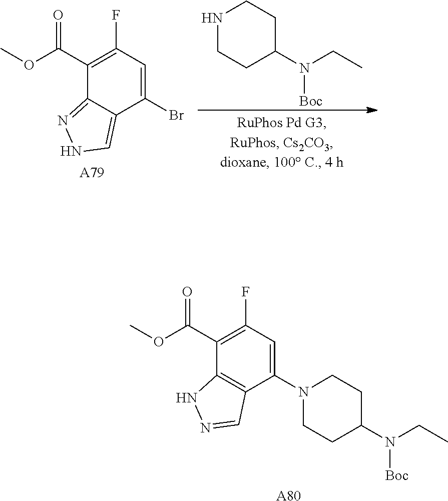

To a stirred mixture of methyl 4-bromo-6-fluoro-2H-indazole-7-carboxylate (A79, 0.5 g, 1.83 mmol) and tert-butyl ethyl (piperidin-4-yl)carbamate (628.3 mg, 2.75 mmol) in dioxane (10 mL) were added Cs₂CO₃ (1.8 g, 5.49 mmol), RuPhos (361.7 mg, 367.2 μmol) and RuPhos Pd G3 (324.6 mg, 180 μmol) at room temperature. The resulting mixture was stirred for 4 h at 100° C. under N₂, allowed to cool to room temperature, diluted with water (30 mL), and extracted with EtOAc (2×20 mL). The combined organic layers were washed with brine (30 mL), dried over anhydrous Na₂SO₄, and filtered. The filtrate was concentrated under reduced pressure and purified by silica gel column chromatography (50% EtOAc in PE) to afford methyl 4-(4-((tert-butoxycarbonyl)(ethyl)amino)piperidin-1-yl)-6-fluoro-1H-indazole-7-carboxylate (A80, 170 mg, 0.40 mmol). LCMS (ES, m/z): 421 [M+H]⁺.

### Synthesis of Intermediate A81

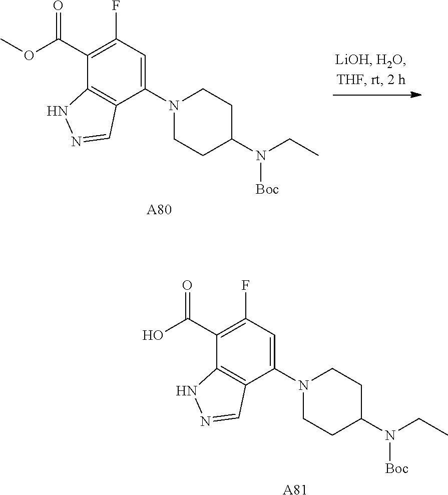 [SMILES: CCN(C(=O)OC(C)(C)C)C1CCN(c2cc(F)c(C(=O)OC)c3[nH]ncc23)CC1 ; "A80" ; CCN(C(=O)OC(C)(C)C)C1CCN(c2cc(F)c(C(=O)O)c3[nH]ncc23)CC1 ; "A81"]

A solution of Intermediate A80 (0.17 g, 0.4 mmol) in THF (3 mL) and H₂O (3 mL) was treated with LiOH (48 mg, 2.0 mmol), and the reaction was stirred for 16 h at room temperature. The resulting mixture was acidified to pH 3 with HCl (1N) and extracted with EtOAc (3×20 mL). The combined organic layers were washed with brine (30 mL), dried over anhydrous Na₂SO₄, filtered, and concentrated under reduced pressure to afford 4-(4-((tert-butoxycarbonyl)-(ethyl)amino)piperidin-1-yl)-6-fluoro-1H-indazole-7-carboxylic acid (A81, 145 mg, 356.6 μmol). LCMS (ES, m/z): 407 [M+H]⁺.

### Synthesis of Intermediate A82

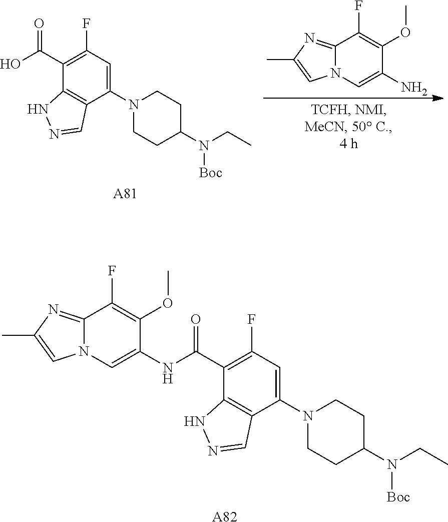

To a stirred mixture of Intermediate A81 (75 mg, 184.5 μmol) and 8-fluoro-7-methoxy-2-methyl-imidazo[1,2-a]pyridin-6-amine (43.2 mg, 221.4 μmol) in MeCN (2 mL) were added NMI (60.6 mg, 738.1 μmol, 58.55 μL) and TCFH (77.6 mg, 276.8 μmol), and the reaction was stirred for 4 h at 50° C. The resulting mixture was diluted with water (10 mL), and extracted with EtOAc (3×15 mL). The combined organic layers were washed with brine (10 mL), dried over anhydrous Na₂SO₄, and filtered. The filtrate was concentrated under reduced pressure and purified by silica gel column chromatography (100% EtOAc) to afford tert-butyl N-ethyl-N-[1-[6-fluoro-7-[(8-fluoro-7-methoxy-2-methyl-imidazo[1,2-a]pyridin-6-yl)carbamoyl]-1H-indazol-4-yl]-4-piperidyl]carbamate (A82, 50 mg). LCMS (ES, m/z): 584 [M+H]⁺.

### Synthesis of Compound 173

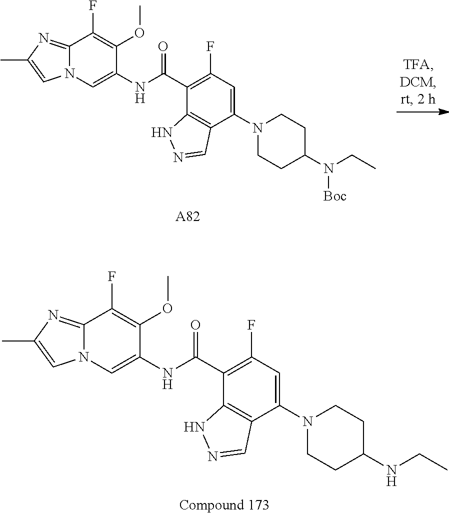

To a stirred mixture of Intermediate A82 (50 mg, 85.67 μmol) in DCM (2 mL) was added TFA (0.5 mL) dropwise, and the reaction was stirred for 2 h at room temperature. The resulting mixture was concentrated under reduced pressure and purified by Prep-HPLC (Condition 5, Gradient 3) to afford 4-[4-(ethylamino)-1-piperidyl]-6-fluoro-N-(8-fluoro-7-methoxy-2-methyl-imidazo[1,2-a]pyridin-6-yl)-1H-indazole-7-carboxamide (Compound 173, 35 mg, 72.4 μmol). LCMS (ES, m/z): 484 [M+H]⁺. ¹H NMR (300 MHz, DMSO-d₆) δ 13.40-12.80 (m, 1H), 9.55-9.16 (m, 1H), 9.18 (s, 1H), 8.31 (s, 1H), 7.95-7.59 (m, 1H), 6.43 (d, J=16.9 Hz, 1H), 4.11 (d, J=2.2 Hz, 3H), 4.01 (d, J=13.0 Hz, 2H), 3.20 (t, J=12.0 Hz, 2H), 2.73-2.71 (m, 1H), 2.61 (q, J=7.1 Hz, 2H), 2.33 (s, 3H), 1.96 (d, J=12.5 Hz, 2H), 1.43 (t, J=11.2 Hz, 2H), 1.04 (t, J=7.1 Hz, 3H).

An analogous method was followed to obtain the following compounds.

| Compound | Starting Material | Characterization |
|---|---|---|
| 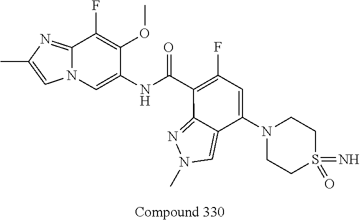 [SMILES: COc1c(NC(=O)c2c(F)cc(N3CCS(=N)(=O)CC3)c3cn(C)nc23)cn2cc(C)nc2c1F] | 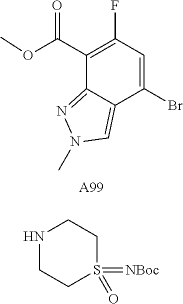 | Modification: Step 1 was ran at 90° C. for 3 h. Step 2 was ran in MeOH and THF. Step 3 was ran at rt for 2 h. The residue was purified by reversed-phase flash chromatography (Con- dition 3, Gradient 11) to afford 6-fluoro-N-(8-fluoro-7-meth- oxy-2-methyl-imidazo[1,2-a]- pyridin-6-yl)-4-(1-imino-1-oxo- 1,4-thiazinan-4-yl)-2-methyl- indazole-7-carboxamide (Compound 330, 0.03 g, 59.6 μmol, 45% yield) as a solid. LCMS (ES, m/z): 504 [M + H]⁺. ¹H NMR (300 MHz, DMSO-d₆) δ 11.73 (s, 1H), 9.68 (s, 1H), 8.88 (s, 1H), 7.99 (d, J = 2.4 Hz, 1H), 6.45 (d, |
| | | J = 15.7 Hz, 1H), 4.33 (d, J = |
| | | 3.4 Hz, 3H), 4.24 (s, 3H), 4.11 |
| | | (d, J = 14.7 Hz, 2H), 3.81 (dd, |
| | | J = 14.5, 6.3 Hz, 2H), 3.25 (t, |
| | | J = 5.0 Hz, 4H), 2.39 (s, 3H). |
| 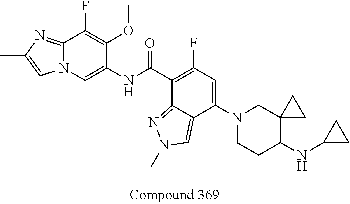 [SMILES: COc1c(NC(=O)c2c(F)cc(N3CCC(NC4CC4)C4(CC4)C3)c3cn(C)nc23)cn2cc(C)nc2c1F] | 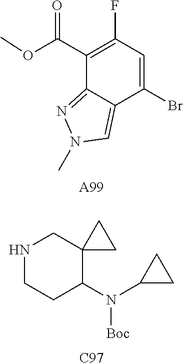 | Modification: Step 1 was ran at 80° C. for 2 h. Step 3 was ran inDMF at room temperature for 16 h. The residue was purified by Prep-HPLC (Condition 5, Gradient 9) to afford 4-[8- (cyclopropylamino)-5-azaspiro [2.5]octan-5-yl]-6-fluoro-N- (8-fluoro-7-methoxy-2-methyl- imidazo[1,2-a]-pyridin-6-yl)- 2-methyl-indazole-7-carbox- amide (Compound 369, 15 mg, 28 μmol, 24% yield) as solid. LCMS (ES, m/z): 536 [M + H]⁺ ¹H NMR (400 MHz, DMSO- d₆) δ 11.56 (s, 1H), 9.46 (d, J = 1.2 Hz, 1H), 8.75 (s, 1H), 7.78- 7.73 (m, 1H), 6.19 (d, J = 16.4 Hz, 1H), 4.23 (s, 3H), 4.18 (d, J = 1.9 Hz, 3H), 3.64-3.56 (m, 3H), 3.08 (d, J = 12.7 Hz, 1H), 2.35 (s, 1H), 2.31 (s, 3H), 2.14- |
| | | 1.98 (m, 3H), 1.84 (s, 1H), 0.58 |
| | | (s, 1H), 0.45-0.36 (m, 5H), 0.26 |
| | | (dq, J = 11.4, 3.8 Hz, 1H). |
| 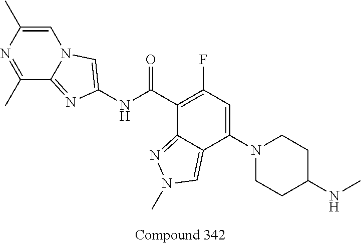 [SMILES: CNC1CCN(c2cc(F)c(C(=O)Nc3cn4cc(C)nc(C)c4n3)c3nn(C)cc23)CC1] | 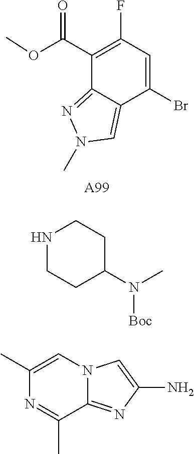 | Modification: Step 1 was ran for 80º C. for 16 h. Step 2 was ran for 16 h. Step 3 was ran at room temperature for 2 h. Step 4 was ran for 1 h. The residue was purified by reversed-phase flash chromatography (Con- dition 3, Gradient 16) to afford N-(6,8-dimethylimidazo[1,2- a]pyrazin-2-yl)-6-fluoro-2- methyl-4-[4-(methylamino)-1- piperidyl]-indazole-7-carbox- amide (Compound 342, 41 mg, 91 μmol, 50% yield) as a solid. LCMS (ES, m/z): 451 [M + H]⁺. ¹H NMR (400 MHz, DMSO-d₆) δ 11.56 (s, 1H), 8.84 (s, 1H), 8.30 (d, J = 9.6 Hz, 2H), 6.28 (d, J = 15.9 Hz, 1H), 4.25 (s, 3H), 3.97 (d, J = 13.1 Hz, 2H), 3.12 (t, J = 12.1 Hz, 2H), 2.82 (s, 1H), 2.69 (s, 3H), 2.40 (d, J = 16.7 Hz, 6H), 2.01 (d, J = 12.6 Hz, 2H), 1.53-1.43 (m, 2H). |
| 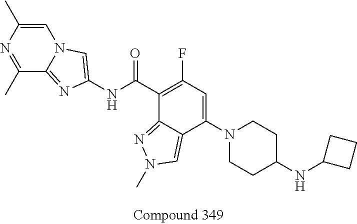 [SMILES: Cc1cn2cc(NC(=O)c3c(F)cc(N4CCC(NC5CCC5)CC4)c4cn(C)nc34)nc2c(C)n1] | 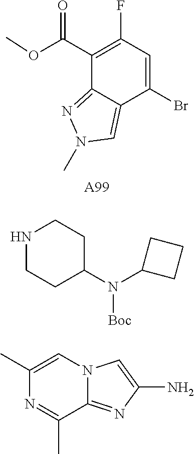 | Modification: Step 1 was ran for 80° C. for 16 h. Sep 2 was ran for 16 h. Step 3 was ran at room temperature for 2 h. The residue was purified by reversed-phase flash chroma- tography (Condition 3, Gradient 16) to afford 4-[4- (cyclobutylamino)-1-piperid- yl]-N-(6,8-dimethyl-imidazo [1,2-a]-pyrazin-2-yl)-6-fluoro- 2-methyl-indazole-7-carbox- amide (Compound 349, 40 mg, 81.5 μmol, 48% yield) as a solid. LCMS (ES, m/z): 491 [M + H]⁺. ¹H NMR (400 MHz, DMSO-d₆) δ 11.56 (s, 1H), 8.82 (s, 1H), 8.30 (d, J = 9.5 Hz, 2H), 6.25 (d, J = 16.0 Hz, 1H), 4.24 (s, 3H), 3.92 (d, J = 13.1 Hz, 2H), 3.10 (t, J = 11.6 Hz, 2H), 2.75 (s, 1H), 2.69 (s, 3H), 2.53 (s, 1H), 2.40-2.35 (m, 3H), 2.14 (d, J = 7.5 Hz, 2H), 1.90 (d, J = 12.6 Hz, 2H), 1.73 (s, 2H), 1.66-1.54 (m, 2H), 1.42 (d, J = 11.8 Hz, 2H). |
| 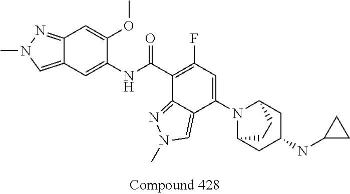 [SMILES: COc1cc2nn(C)cc2cc1NC(=O)c1c(F)cc(N2[C@@H]3CC[C@H]2C[C@@H](NC2CC2)C3)c2cn(C)nc12] | 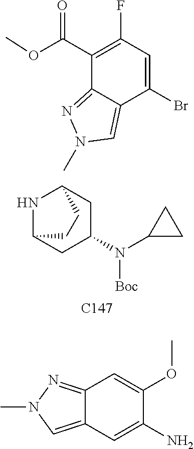 | Modification: Step 1 was ran at 75° C. for 2 h. Step 2 was ran in H₂O, THF, and MeOH. Step 3 was ran for 2 h at room temperature. The crude product was purified by Prep-HPLC (Condition 5, Gradient 10) to afford 4-[(1R,5S)-3-(cyclo- propyl-amino)-8-azabicyclo [3.2.1]octan-8-yl]-6-fluoro-N- (6-methoxy-2-methyl-indazol- 5-yl)-2-methyl-indazole-7- carboxamide (Compound 428, 12 mg, 23.2 μmol, 26% yield) as a solid. LCMS (ES, m/z): 518 [M + H]⁻. ¹H NMR (400 MHz, DMSO-d₆) δ 11.58 (s, 1H), 8.85 (s, 1H), 8.76 (s, 1H), 8.13 (s, 1H), 7.02 (s, 1H), 6.10 (d, J = 16.6 Hz, 1H), 4.52 (s, 2H), 4.20 (s, 3H), 4.06 (s, 3H), 4.00 (s, 3H), 2.91 (d, J = 5.9 Hz, 1H), 2.21 (d, J = 6.9 Hz, 3H), 2.02-1.87 (m, 5H), 1.77 (d, J = 14.2 Hz, 2H), 0.40 (dt, J = 6.2, 2.9 Hz, 2H), 0.28 (q, J = 3.3, 2.7 Hz, 2H). |
| 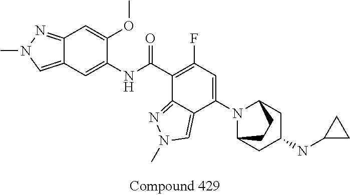 [SMILES: COc1cc2nn(C)cc2cc1NC(=O)c1c(F)cc(N2[C@@H]3CC[C@H]2C[C@H](NC2CC2)C3)c2cn(C)nc12] | 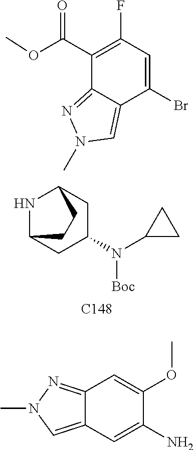 | Modification: Step 1 was ran at 90° C. for 2 h. Step 2 was ran in H₂O, THF, and MeOH. Step 3 was ran for 2 h at room temperature. Step 4 was ran for 1 h. The residue was purified by reversed-phase flash chromatography (Con- dition 3, Gradient 18) to afford 4-[(1S,5R)-3-(cyclopropyl- amino)-8-azabicyclo-[3.2.1] octan-8-yl]-6-fluoro-N-(6- methoxy-2-methyl-indazol-5- yl)-2-methyl-indazole-7- carboxamide (Compound 429, 30 mg, 57.96 μmol, 36% yield) as a solid. LCMS (ES, m/z): 518 [M + H]⁺ ¹H NMR (400 MHz, DMSO-d₆) δ 11.60 (s, 1H), 8.88 (s, 1H), 8.78 (s, 1H), 8.16 (s, 1H), 7.04 (s, 1H), 6.16 (d, J = 16.5 Hz, 1H), 4.63 (s, 2H), 4.23 (s, 3H), 4.08 (s, 3H), 4.03 (s, 3H), 2.03 (d, J = 10.1 Hz, 4H), 1.87 (dd, J = 14.1, 8.3 Hz, 4H), 1.42 (t, J = 11.9 Hz, 2H), 0.35-0.26 (m, 2H), 0.16-0.08 (m, 2H). |

### Example 18: Synthesis of Compound 174

### Synthesis of Intermediate A83

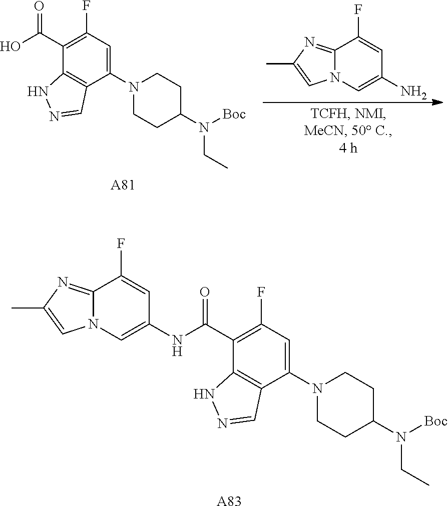 [SMILES: CCN(C(=O)OC(C)(C)C)C1CCN(c2cc(F)c(C(=O)O)c3[nH]ncc23)CC1 ; Cc1cn2cc(N)cc(F)c2n1 ; "A81" ; CCN(C(=O)OC(C)(C)C)C1CCN(c2cc(F)c(C(=O)Nc3cc(F)c4nc(C)cn4c3)c3[nH]ncc23)CC1 ; "A83"]

To a stirred mixture of Intermediate A83 (70 mg, 172.2 μmol) and 8-fluoro-2-methyl-imidazo[1,2-a]pyridin-6-amine (34.1 mg, 206.7 μmol) in MeCN (1.5 mL) were added NMI (56.5 mg, 688.9 μmol, 54.65 μL) and TCFH (72.4 mg, 258.3 μmol) at room temperature. The resulting mixture was stirred for 4 h at 50° C., diluted with water (10 mL), and then extracted with EtOAc (3×10 mL). The combined organic layers were washed with water (3×15 mL) and brine (15 mL), dried over anhydrous Na₂SO₄, and filtered. The filtrate was concentrated under reduced pressure and purified by silica gel column chromatography (100% EtOAc) to afford tert-butyl N-ethyl-N-[1-[6-fluoro-7-[(8-fluoro-2-methyl-imidazo[1,2-a]pyridin-6-yl)carbamoyl]-1H-indazol-4-yl]-4-piperidyl]carbamate (A83, 66 mg, 101.2 μmol). LCMS (ES, m/z): 554 [M+H]⁺.

### Synthesis of Compound 174

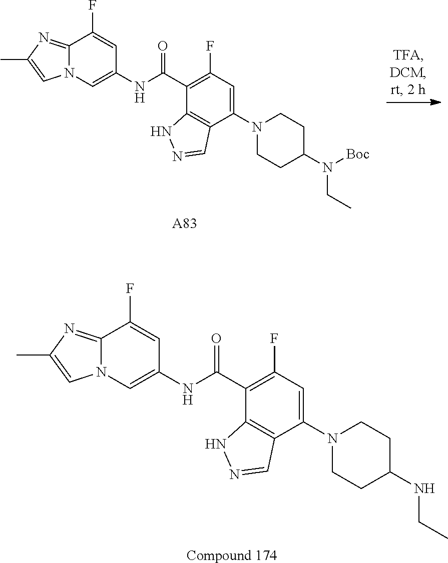 [SMILES: CCN(C(=O)OC(C)(C)C)C1CCN(c2cc(F)c(C(=O)Nc3cc(F)c4nc(C)cn4c3)c3[nH]ncc23)CC1 ; "A83" ; CCNC1CCN(c2cc(F)c(C(=O)Nc3cc(F)c4nc(C)cn4c3)c3[nH]ncc23)CC1 ; "Compound 174"]

To a stirred mixture of Intermediate A83 (65 mg, 92.12 μmol) in DCM (2 mL) was added TFA (0.5 mL), and the reaction was stirred for 2 h at room temperature. The resulting mixture was concentrated under reduced pressure and purified by Prep-HPLC (Condition 5, Gradient 4) to afford 4-[4-(ethylamino)-1-piperidyl]-6-fluoro-N-(8-fluoro-2-methyl-imidazo-[1,2-a]pyridin-6-yl)-1H-indazole-7-carboxamide (Compound 174, 50 mg, 66.15 μmol). LCMS (ES, m/z): 454 [M+H]⁺. ¹H NMR (300 MHz, DMSO-d₆) δ 13.50-12.50 (m, 1H), 9.96 (s, 1H), 9.03 (d, J=1.6 Hz, 1H), 8.21 (s, 1H), 7.91 (d, J=3.1 Hz, 1H), 7.32 (dd, J=12.7, 1.6 Hz, 1H), 6.39 (d, J=15.2 Hz, 1H), 3.95 (d, J=13.2 Hz, 2H), 3.14 (t, J=11.9 Hz, 2H), 2.72 (d, J=9.9 Hz, 1H), 2.61 (q, J=7.1 Hz, 2H), 2.35 (s, 3H), 1.96 (d, J=12.7 Hz, 2H), 1.42 (q, J=10.3, 9.6 Hz, 2H), 1.04 (t, J=7.1 Hz, 3H).

An analogous method was followed, using B90-a in place of B9, to obtain the following compounds.

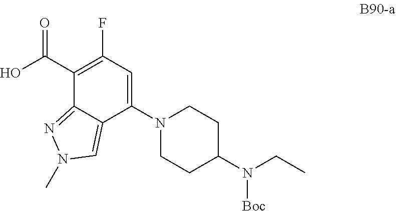 [SMILES: CCN(C(=O)OC(C)(C)C)C1CCN(c2cc(F)c(C(=O)O)c3nn(C)cc23)CC1]

| Compound | Starting Material | Characterization |
|---|---|---|
| 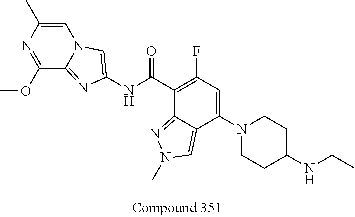 [SMILES: CCNC1CCN(c2cc(F)c(C(=O)Nc3cn4cc(C)nc(OC)c4n3)c3nn(C)cc23)CC1] | 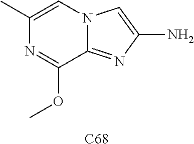 | Modification: Step 1 was ran at room temperature. The residue was purified by reversed-phase flash chromatography (Condition 3, Gradient 11) to afford 4-[4-(ethyl- amino)-1-piperidyl]-6- fluoro-N-(8-methoxy-6- methyl-imidazo[1,2-a]- pyrazin-2-yl)-2-methyl- indazole-7-carboxamide (Compound 351, 20 mg, 41.6 μmol, 40% yield) as |
| | | a solid. LCMS (ES, m/z): |
| | | 481 [M + H]⁺ ¹H NMR |
| | | (400 MHz, DMSO-d₆) δ |
| | | 11.58 (s, 1H), 8.81 (s, |
| | | 1H), 8.23 (s, 1H), 8.08 |
| | | (d, J = 1.3 Hz, 1H), 6.24 |
| | | (d, J = 16.0 Hz, 1H), |
| | | 4.24 (s, 3H), 4.03 (s, |
| | | 3H), 3.92 (d, J = 12.9 Hz, |
| | | 2H), 3.18-3.07 (m, 2H), |
| | | 2.62 (d, J = 7.1 Hz, 3H), |
| | | 2.57 (d, J = 17.2 Hz, 1H), |
| | | 2.31 (d, J = 1.0 Hz, 3H), |
| | | 1.94 (d, J = 12.2 Hz, 2H), |
| | | 1.41 (q, J = 9.7 Hz, 2H), |
| | | 1.03 (t, J = 7.1 Hz, 3H). |
| 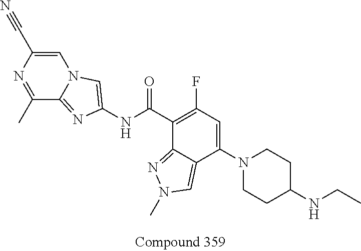 [SMILES: CCNC1CCN(c2cc(F)c(C(=O)Nc3cn4cc(C#N)nc(C)c4n3)c3nn(C)cc23)CC1] | 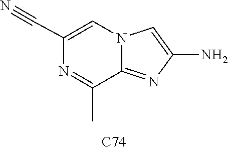 | Modification: Step 1 was ran for 24 h. The residue was purified by reversed- phase flash chroma- tography (Condition 3, Gradient 6) to afford N- (6-cyano-8-methyl- imidazo-[1,2-a]pyrazin- 2-yl)-4-[4-(ethylamino)- 1-piperidyl]-6-fluoro-2- methyl-indazole-7- carboxamide (Compound 359, 19 mg, 39.96 μmol, 23% yield) as a solid. LCMS (ES, m/z): 476 [M + H]⁺. ¹H NMR (300 |
| | | MHz, DMSO-d₆) δ 11.76 |
| | | (s, 1H), 9.34 (s, 1H), 8.84 |
| | | (s, 1H), 8.48 (s, 1H), 6.25 |
| | | (d, J = 16.1 Hz, 1H), 4.24 |
| | | (s, 3H), 3.94 (d, J = 13.1 |
| | | Hz, 2H), 3.15 (t, J = 11.9 |
| | | Hz, 2H), 2.76 (s, 4H), |
| | | 2.60 (q, J = 7.1 Hz, 2H), |
| | | 1.95 (d, J = 12.9 Hz, 2H), |
| | | 1.67 (s, 1H), 1.40 (q, J = |
| | | 10.7, 10.3 Hz, 2H), 1.04 |
| | | (t, J = 7.1 Hz, 3H). |
| 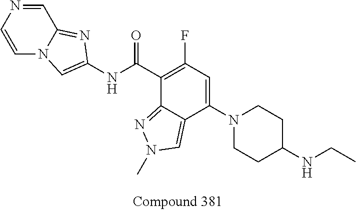 [SMILES: CCNC1CCN(c2cc(F)c(C(=O)Nc3cn4ccncc4n3)c3nn(C)cc23)CC1] | 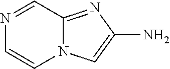 | Modification: Step 1 was ran in DCM at room temperature. The crude product was purified by Prep-HPLC (Condition 5, Gradient 11) to afford 4- [4-(ethylamino)-1-piper- idyl]-6-fluoro-N-imidazo [1,2-a]-pyrazin-2-yl-2- methyl-indazole-7- carboxamide (Compound 381, 16 mg, 36.7 μmol, 28% yield) as a solid. |
| | | LCMS (ES, m/z): 437 |
| | | [M + H]⁻. ¹H NMR (400 |
| | | MHz, DMSO-d₆) δ 11.77 |
| | | (s, 1H), 8.94 (d, J = 1.4 |
| | | Hz, 1H), 8.83 (s, 1H), |
| | | 8.64 (dd, J = 4.4, 1.5 Hz, |
| | | 1H), 8.43 (s, 1H), 7.90 (d, |
| | | J = 4.5 Hz, 1H), 6.25 (d, |
| | | J = 16.1 Hz, 1H), 4.24 (s, |
| | | 3H), 3.93 (d, J = 13.1 Hz, |
| | | 2H), 3.20-3.03 (m, 2H), |
| | | 2.70 (td, J = 9.2, 8.8, 4.3 |
| | | Hz, 1H), 2.60 (q, J = 7.1 |
| | | Hz, 2H), 1.95 (d, J = 12.8 |
| | | Hz, 2H), 1.48-1.32 (m, |
| | | 2H), 1.04 (t, J = 7.1 Hz, |
| | | 3H). |
| 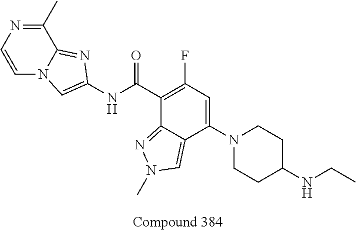 [SMILES: CCNC1CCN(c2cc(F)c(C(=O)Nc3cn4ccnc(C)c4n3)c3nn(C)cc23)CC1] | 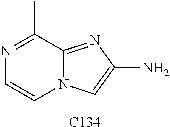 | Modification: Step 1 was ran at 40° C. for 24 h. The mixture was purified by reversed-phase flash chromatography (Con- dition 3, Gradient 19) to afford 4-[4-(ethylamino)- 1-piperidyl]-6-fluoro-2- methyl-N-(8-methyl- imidazo[1,2-a]-pyrazin- 2-yl)-indazole-7-carbox- amide (Compound 384, 0.04 g, 88.8 μmol, 49% yield) as a solid. LCMS (ES, m/z): 451 [M + H]⁺. |
| | | ¹H NMR (400 MHz, |
| | | DMSO-d₆) δ 11.59 (s, |
| | | 1H), 8.80 (s, 1H), 8.46 (d, |
| | | J = 4.6 Hz, 1H), 8.37 (s, |
| | | 1H), 7.72 (d, J = 4.5 Hz, |
| | | 1H), 6.22 (d, J = 16.0 Hz, |
| | | 1H), 4.22 (s, 3H), 3.90 |
| | | (d, J = 13.0 Hz, 2H), 3.11 |
| | | (t, J = 11.2 Hz, 2H), 2.70 |
| | | (s, 3H), 2.67 (s, 1H), 2.58 |
| | | (q, J = 7.1 Hz, 2H), 1.93 |
| | | (d, J = 12.9 Hz, 2H), |
| | | 1.45-1.31 (m, 2H), 1.01 |
| | | (t, J = 7.1 Hz, 3H). |
| 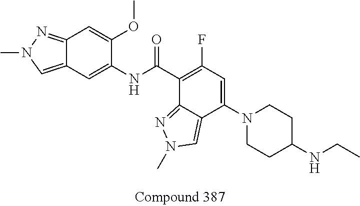 [SMILES: CCNC1CCN(c2cc(F)c(C(=O)Nc3cc4cn(C)nc4cc3OC)c3nn(C)cc23)CC1] | 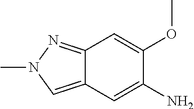 | Modification: Step 1 was ran at room temperature for 16 h. Step 2 was ran using HCl in dioxane and DCM at room temperature. The crude product was purified by Prep-HPLC (Condition 5, Gradient 7a) to afford 4-[4-(ethyl- amino)-1-piperidyl]-6- fluoro-N-(6-methoxy-2- methyl-indazol-5-yl)-2- methyl-indazole-7- |
| | | carboxamide (Compound |
| | | 387, 28 mg, 58.4 μmol, |
| | | 42% yield) as a solid. |
| | | LCMS (ES, m/z): 480 |
| | | [M + H]⁺. ¹H NMR (400 |
| | | MHz, DMSO-d₆) δ 11.59 |
| | | (s, 1H), 8.78 (d, J = 2.0 |
| | | Hz, 2H), 8.17 (s, 1H), |
| | | 7.05 (s, 1H), 6.21 (d, J = |
| | | 16.0 Hz, 1H), 4.24 (s, |
| | | 3H), 4.08 (s, 3H), 4.03 |
| | | (s, 3H), 3.90 (d, J = 12.9 |
| | | Hz, 2H), 3.10 (t, J = 11.5 |
| | | Hz, 2H), 2.69 (s, 1H), |
| | | 2.61 (q, J = 7.1 Hz, 2H), |
| | | 1.95 (d, J = 12.2 Hz, 2H), |
| | | 1.43-1.39 (m, 2H), 1.04 |
| | | (t, J = 7.1 Hz, 3H). |

An analogous method was followed, using B101 in place of B9, to obtain the following compounds.

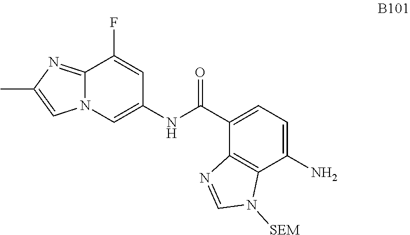 [SMILES: Cc1cn2cc(NC(=O)c3ccc(N)c4c3ncn4COCC[Si](C)(C)C)cc(F)c2n1]

| Compound | Starting Material | Characterization |
|---|---|---|
| 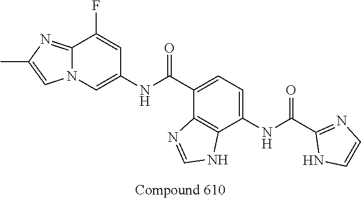 [SMILES: Cc1cn2cc(NC(=O)c3ccc(NC(=O)c4ncc[nH]4)c4[nH]cnc34)cc(F)c2n1] | 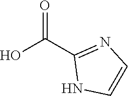 | Modification: Step 1 was ran at room temperature for 16 h. The residue was purified by Prep-HPLC (Condition 2, Gradient 4) to afford N-(8-fluoro-2- methylimidazo[1,2-a]pyridin-6-yl)-7- (1H-imidazole-2-carboxamido)-1- ((2-(trimethylsilyl)ethoxy)-methyl)- 1H-benzo[d]imidazole-4-carboxamide 2,2,2-trifluoroacetate (Compound 610, 50 mg, 75.45 μmol, 69% yield) as a solid. LCMS (ES, m/z): 419 [M + H]⁻. ¹H NMR (400 MHz, DMSO-d₆) δ 11.24 (s, 1H), 10.53 (s, 1H), 9.36 (s, |
| | | 1H), 8.48 (s, 1H), 8.20 (s, 1H), 8.15 |
| | | (d, J = 8.4 Hz, 1H), 8.04 (d, J = 8.4 |
| | | Hz, 1H), 7.87 (d, J = 12.0 Hz, 1H), |
| | | 7.37 (s, 2H), 2.46 (s, 3H). |
| 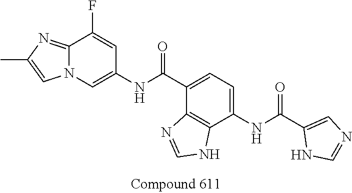 [SMILES: Cc1cn2cc(NC(=O)c3ccc(NC(=O)c4cnc[nH]4)c4[nH]cnc34)cc(F)c2n1] | 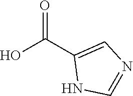 | The residue was purified by Prep- HPLC (Condition 2, Gradient 4) to afford N-(8-fluoro-2-methylimidazo [1,2-a]pyridin-6-yl)-7-(1H-imidazole- 5-carboxamido)-1-((2-(trimethylsilyl) ethoxy)-methyl)-1H-benzo[d]-imid- azole-4-carboxamide bis(2,2,2- trifluoroacetate) (Compound 611, 33 mg, 42.5 μmol, 31% yield) as a solid. LCMS (ES, m/z): 419 [M + H]⁺. ¹H NMR (400 MHz, DMSO-d₆) δ 11.10 (s, 1H), 10.43 (s, 1H), 9.36 (s, 1H), |
| | | 8.43 (s, 1H), 8.21 (d, J = 2.5 Hz, 1H), |
| | | 8.16 (s, 1H), 8.08 (s, 1H), 8.06-7.99 |
| | | (m, 2H), 7.88 (d, J = 12.0 Hz, 1H), |
| | | 2.47 (s, 3H). |

An analogous method was followed, beginning with B97, to obtain the following compounds.

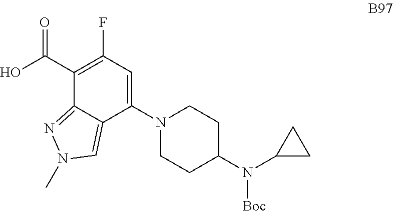 [SMILES: Cn1cc2c(N3CCC(N(C(=O)OC(C)(C)C)C4CC4)CC3)cc(F)c(C(=O)O)c2n1]

| Compound | Starting Material | Characterization |
|---|---|---|
| 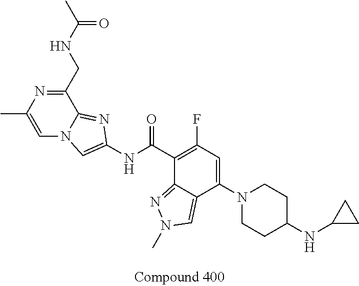 [SMILES: CC(=O)NCc1nc(C)cn2cc(NC(=O)c3c(F)cc(N4CCC(NC5CC5)CC4)c4cn(C)nc34)nc12] | 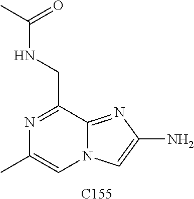 | The resulting mixture was concentrated under reduced pressure and lyophilized to afford N-(8-(acetamido- methyl)-6-methylimidazo [1,2-a]-pyrazin-2-yl)-4-(4- (cyclopropylamino)-piper- idin-1-yl)-6-fluoro-2-meth- yl-2H-indazole-7-carbox- amide 2,2,2-trifluoro- acetate (Compound 400, 4.4 mg, 6.8 μmol, 29% yield) as a solid. LCMS (ES, m/z): 534 [M + H]⁺. ¹H NMR (400 MHz, DMSO-d₆) δ 11.58 (s, 1H), 8.87 (s, 1H), 8.69 (s, 2H), 8.40-8.30 (m, 3H), |
| | | 6.34 (d, J = 15.6 Hz, 1H), |
| | | 4.69 (d, J = 5.5 Hz, 2H), |
| | | 4.24 (s, 3H), 4.08 (d, J = |
| | | 13.2 Hz, 2H), 3.80-3.70 |
| | | (m, 1H), 3.09 (t, J = 12.5 |
| | | Hz, 2H), 2.77 (s, 1H), |
| | | 2.40 (d, J = 1.0 Hz, 3H), |
| | | 2.16 (d, J = 12.2 Hz, 2H), |
| | | 1.91 (s, 3H), 1.71-1.66 |
| | | (m, 2H), 0.86-0.75 (m, |
| | | 4H). |
| 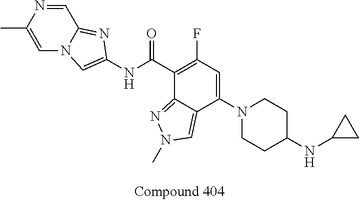 [SMILES: Cc1cn2cc(NC(=O)c3c(F)cc(N4CCC(NC5CC5)CC4)c4cn(C)nc34)nc2cn1] | 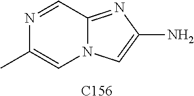 | Modification: Step 1 was ran at room temperature to 40° C. for 16 h. The crude product was purified by Prep-HPLC (Condition 2, Gradient 11) to afford 4- (4-(cyclopropylamino)- piperidin-1-yl)-6-fluoro-2- methyl-N-(6-methylimid- azo-[1,2-a]pyrazin-2-yl)- 2H-indazole-7-carbox- amide 2,2,2-trifluoro- acetate (Compound 404, |
| | | 50 mg, 86.7 μmol, 61% |
| | | yield) as a solid. LCMS |
| | | (ES, m/z): 463 [M + H]⁺. |
| | | ¹H NMR (400 MHz, |
| | | DMSO-d₆) δ 11.74 (s, |
| | | 1H), 8.90 (d, J = 2.0 Hz, |
| | | 2H), 8.78 (d, J = 6.7 Hz, |
| | | 2H), 8.51 (t, J = 1.3 Hz, |
| | | 1H), 8.38 (s, 1H), 6.36 (d, |
| | | J = 15.7 Hz, 1H), 4.25 (s, |
| | | 3H), 4.11 (d, J = 13.2 Hz, |
| | | 1H), 3.51 (s, 1H), 3.12 (t, |
| | | J = 12.6 Hz, 2H), 2.80 (d, |
| | | J = 7.6 Hz, 1H), 2.48-2.43 |
| | | (m, 3H), 2.19 (d, J = 12.3 |
| | | Hz, 2H), 1.81-1.62 (m, |
| | | 2H), 0.89-0.79 (m, 4H). |
| 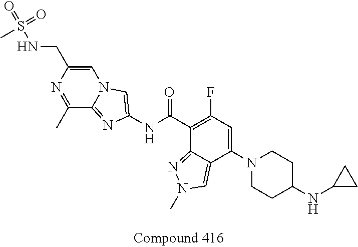 [SMILES: Cc1nc(CNS(C)(=O)=O)cn2cc(NC(=O)c3c(F)cc(N4CCC(NC5CC5)CC4)c4cn(C)nc34)nc12] | 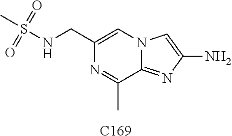 | Modification: Step 1 was ran for 16 h. The crude product was purified by Prep-HPLC (Condition 7, Gradient 1) to afford 4-(4- (cyclopropylamino)-piper- idin-1-yl)-6-fluoro-2-meth- yl-N-(8-methyl-6-(methyl- sulfonamidomethyl)- imidazo[1,2-a]pyrazin-2- yl)-2H-indazole-7-carbox- amide 2,2,2-trifluoroacetate (Compound 416, 17 mg, 24.9 μmol, 76% yield) as a solid. LCMS (ES, m/z): 570 [M + H]⁺. ¹H NMR |
| | | (400 MHz, DMSO-d₆) δ |
| | | 11.59 (s, 1H), 8.89 (s, 1H), |
| | | 8.78 (s, 2H), 8.49 (s, 1H), |
| | | 8.44 (s, 1H), 7.62 (t, J = 6.2 |
| | | Hz, 1H), 6.36 (d, J = 15.6 |
| | | Hz, 1H), 4.26 (s, 3H), 4.23 |
| | | (d, J = 6.0 Hz, 2H), 4.10 |
| | | (d, J = 13.3 Hz, 2H), 3.51 |
| | | (s, 1H), 3.12 (t, J = 12.6 |
| | | Hz, 2H), 3.00 (s, 3H), |
| | | 2.83-2.76 (m, 1H), 2.73 (s, |
| | | 3H), 2.19 (d, J = 11.2 Hz, |
| | | 2H), 1.79-1.65 (m, 2H), |
| | | 0.88-0.81 (m, 4H). |
| 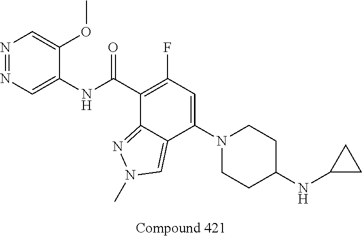 [SMILES: COc1cnncc1NC(=O)c1c(F)cc(N2CCC(NC3CC3)CC2)c2cn(C)nc12] | 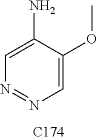 | Modification: Step 1 was ran for 2 h at room temperature. The resulting mixture was purified by reversed-phase flash chromatography (Condition 3, Gradient 10) to afford 4- [4-(cyclopropylamino)-1- piperidyl]-6-fluoro-N-(5- methoxypyridazin-4-yl)-2- methyl-indazole-7-carbox- amide (Compound 421, 0.02 g, 47.3 μmol, 32% yield) as a solid. LCMS (ES, m/z): 440 [M + H]⁺. |
| | | ¹H NMR (400 MHz, |
| | | DMSO-d₆) δ 11.85 (s, 1H), |
| | | 10.21 (s, 1H), 9.14 (s, 1H), |
| | | 8.84 (s, 1H), 6.25 (d, J = |
| | | 16.5 Hz, 1H), 4.21 (s, 3H), |
| | | 4.17 (s, 3H), 4.00-3.91 (m, |
| | | 2H), 3.18 (ddd, J = 13.4, |
| | | 11.0, 2.8 Hz, 2H), 2.82 (dt, |
| | | J = 9.7, 5.5 Hz, 1H), 2.12 |
| | | (tt, J = 6.7, 3.6 Hz, 1H), |
| | | 2.03-1.94 (m, 2H), 1.45- |
| | | 1.42 (m, 1H), 0.40 (td, J = |
| | | 6.3, 4.1 Hz, 2H), 0.27-0.19 |
| | | (m, 2H) |

An analogous method was followed to obtain the following compounds.

| Compound | Starting Material | Characterization |
|---|---|---|
| 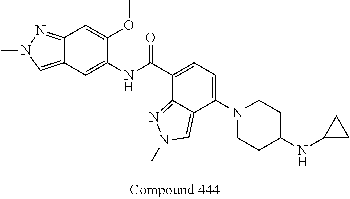 [SMILES: COc1cc2nn(C)cc2cc1NC(=O)c1ccc(N2CCC(NC3CC3)CC2)c2cn(C)nc12] | 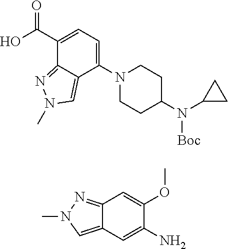 | Modification: Step 1 was ran at room temperature for 2 h. Step 2 was ran for 2 h at room temperature. The crude product was purified by Prep-HPLC (Condition 6, Gradient 7) to afford 4- [4-(cyclopropylamino)-1- piperidyl]-N-(6-methoxy- 2-methyl-indazol-5-yl)-2- methyl-indazole-7-carbox- amide (Compound 444, 32.7 mg, 69.1 μmol, 57% yield) as a solid. LCMS (ES, m/z): 474 [M + H]⁻. |
| | | ¹H NMR (400 MHz, |
| | | DMSO-d₆) δ 11.62 (s, 1H), |
| | | 8.84 (s, 1H), 8.74 (s, 1H), |
| | | 8.18 (s, 1H), 7.99 (d, J = |
| | | 8.0 Hz, 1H), 7.07 (s, 1H), |
| | | 6.48 (d, J = 8.1 Hz, 1H), |
| | | 4.29 (s, 3H), 4.08 (s, 3H), |
| | | 4.07 (s, 3H), 3.87 (d, J = |
| | | 12.6 Hz, 2H), 3.03 (t, J = |
| | | 11.3 Hz, 2H), 2.81 (s, 1H), |
| | | 2.17 (s, 1H), 2.06-1.98 (m, |
| | | 2H), 1.50 (q, J = 11.1, 10.5 |
| | | Hz, 2H), 0.43 (dt, J = 6.3, |
| | | 3.1 Hz, 2H), 0.28 (d, J = |
| | | 3.3 Hz, 2H). |
| 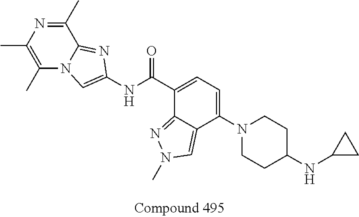 [SMILES: Cc1nc(C)c2nc(NC(=O)c3ccc(N4CCC(NC5CC5)CC4)c4cn(C)nc34)cn2c1C] | 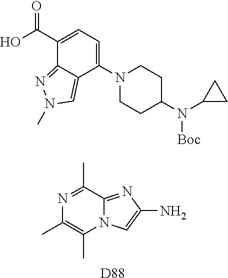 | Modification: Step 1 was ran for 2 h at room temperature. The crude product was purified by Prep-HPLC (Condition 5, Gradient 19b) to afford 4-[4-(cyclopropyl- amino)-1-piperidyl]-2-meth- yl-N-(5,6,8-trimethylimi- dazo-[1,2-a]pyrazin-2-yl)- indazole-7-carboxamide (Compound 495, 27 mg, 57.1 μmol). LCMS (ES, m/z): 473 [M + H]⁺. ¹H NMR (400 MHz, DMSO- d₆) δ 11.47 (s, 1H), 8.80 (s, 1H), 8.17 (s, 1H), 8.04 (d, J = 8.1 Hz, 1H), 6.51 (d, |
| | | J = 8.2 Hz, 1H), 4.31 (s, |
| | | 3H), 3.90 (d, J = 12.8 Hz, |
| | | 2H), 3.08 (t, J = 11.4 Hz, |
| | | 2H), 2.78 (d, J = 10.0 Hz, |
| | | 1H), 2.69 (s, 3H), 2.57 (s, |
| | | 3H), 2.47 (s, 3H), 2.20- |
| | | 2.09 (m, 1H), 2.01 (d, J = |
| | | 12.6 Hz, 2H), 1.48 (q, J = |
| | | 11.5 Hz, 2H), 0.41 (dt, J = |
| | | 6.3, 3.1 Hz, 2H), 0.29-0.20 |
| | | (m, 2H). |
| 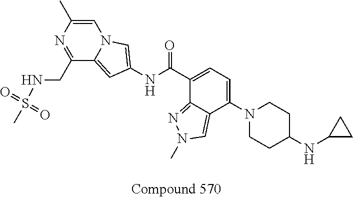 [SMILES: Cc1cn2cc(NC(=O)c3ccc(N4CCC(NC5CC5)CC4)c4cn(C)nc34)cc2c(CNS(C)(=O)=O)n1] | 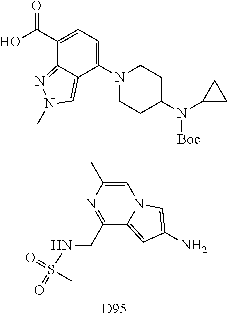 [SMILES: Cn1cc2c(N3CCC(N(C(=O)OC(C)(C)C)C4CC4)CC3)ccc(C(=O)O)c2n1 ; Cc1cn2cc(N)cc2c(CNS(C)(=O)=O)n1 ; "D95"] | Modifications: Step 1 was ran at room temperature for 3 h. Step 2 was ran for 1 h. The crude product was purified by Prep-HPLC (Condition 5, Gradient 31) to afford 4-[4-(cyclopropyl- amino)-1-piperidyl]-N-[1- (methanesulfonamidometh- yl)-3-methyl-pyrrolo[1,2-a]- pyrazin-7-yl]-2-methyl- indazole-7-carboxamide (Compound 570, 9.0 mg, 15.6 μmol). LCMS (ES, m/z): 551 [M + H]⁺. ¹H NMR (400 MHz, DMSO- d₆) δ 11.21 (s, 1H), 8.77 (s, 1H), 8.29 (d, J = 1.4 Hz, 1H), 8.11 (s, 1H), 7.99 (d, |
| | | J = 8.0 Hz, 1H), 7.54 (t, J = |
| | | 6.0 Hz, 1H), 6.97 (s, 1H), |
| | | 6.49 (d, J = 8.2 Hz, 1H), |
| | | 4.45 (d, J = 5.7 Hz, 2H), |
| | | 4.30 (s, 3H), 3.87 (d, J = |
| | | 12.6 Hz, 2H), 3.05 (t, J = |
| | | 11.7 Hz, 2H), 2.99 (s, 3H), |
| | | 2.78 (t, J = 9.7 Hz, 1H), |
| | | 2.33 (s, 3H), 2.28 (s, 1H), |
| | | 2.21-2.08 (m, 1H), 2.01 (d, |
| | | J = 12.5 Hz, 2H), 1.48 (q, |
| | | J = 10.3 Hz, 2H), 0.40 (dt, |
| | | J = 6.2, 3.0 Hz, 2H), 0.24 |
| | | (p, J = 3.9 Hz, 2H). |
| 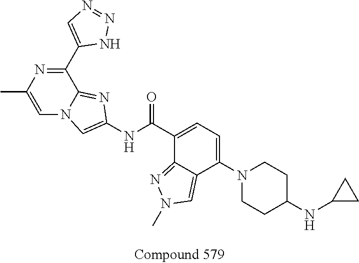 [SMILES: Cc1cn2cc(NC(=O)c3ccc(N4CCC(NC5CC5)CC4)c4cn(C)nc34)nc2c(-c2cnn[nH]2)n1] | 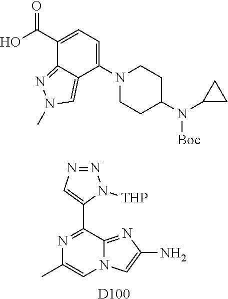 | Modification: Step 1 was ran for 6 h at room temperature. Step 2 was ran for 1 h. The residue was purified by reversed-phase flash chromatography (Condition 10, Gradient 1) to afford 4- (4-(cyclopropylamino)piper- idin-1-yl)-2-methyl-N-(6- methyl-8-(1H-1,2,3-triazol- 5-yl)-imidazo[1,2-a]pyrazin- 2-yl)-2H-indazole-7-carbox- amide 2,2,2-trifluoroacetate (Compound 579, 6 mg, 9.6 μmol). LCMS (ES, m/z): 512 [M + H]⁺. ¹H NMR (400 MHz, DMSO-d₆) δ 11.64 (s, 1H), 8.89 (s, 2H), |
| | | 8.77 (s, 2H), 8.53 (s, 1H), |
| | | 8.48 (s, 1H), 8.08 (d, J = |
| | | 8.0 Hz, 1H), 6.59 (d, J = 8.1 |
| | | Hz, 1H), 4.36 (s, 3H), 4.08 |
| | | (d, J = 13.0 Hz, 2H), 3.50 |
| | | (s, 1H), 3.08 (t, J = 12.4 Hz, |
| | | 2H), 2.80 (s, 1H), 2.52 (s, |
| | | 3H), 2.21 (d, J = 11.8 Hz, |
| | | 2H), 1.89-1.70 (m, 2H), |
| | | 0.89-0.82 (m, 4H). |
| 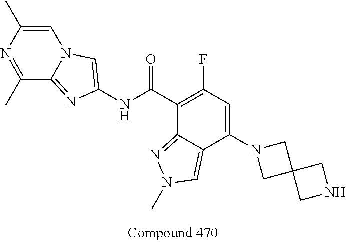 [SMILES: Cc1cn2cc(NC(=O)c3c(F)cc(N4CC5(CNC5)C4)c4cn(C)nc34)nc2c(C)n1] | 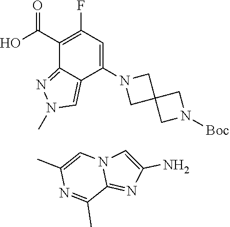 | Modification: Step 1 was ran at room temperature for 3 h. Step 2 was ran for 3 h at room temperature. The crude product was purified by Prep-HPLC (Condition 2, Gradient 14) to afford 4- (2,6-diazaspiro[3.3]heptan- 2-yl)-N-(6,8-dimethyl- imidazo-[1,2-a]-pyrazin- 2-yl)-6-fluoro-2-methyl- indazole-7-carboxamide (Compound 470, 6.4 mg, 14.7 μmol). LCMS (ES, m/z): 435 [M + H]⁺. ¹H |
| | | NMR (400 MHz,Methanol- |
| | | d₄) δ 8.39 (s, 1H), 8.27 (s, |
| | | 1H), 8.17 (s, 1H), 5.66 (d, |
| | | J = 15.1 Hz, 1H), 4.39 (s, |
| | | 4H), 4.29 (s, 3H), 3.89 (s, |
| | | 4H), 2.78 (s, 3H), 2.44 (s, |
| | | 3H). |
| 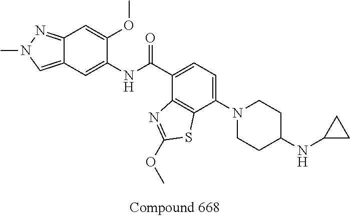 [SMILES: COc1nc2c(C(=O)Nc3cc4cn(C)nc4cc3OC)ccc(N3CCC(NC4CC4)CC3)c2s1] | 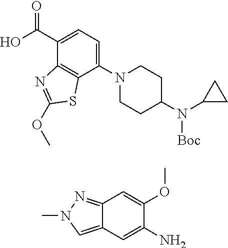 | Modification: Step 1 was ran for 2 h at room temperature. The residue was purified by reversed-phase flash chromatography (Condition 6, Gradient 1) to afford 7- (4-(cyclopropylamino)- piperidin-1-yl)-2-methoxy- N-(6-methoxy-2-methyl- 2H-indazol-5-yl)benzo[d]- thiazole-4-carboxamide 2,2,2-trifluoroacetate (Compound 668, 0.01 g, 12.9 μmol). LCMS (ES, m/z): 507 [M + H]⁺. ¹H |
| | | NMR (300 MHz, DMSO- |
| | | d₆) δ 11.4 (s, 1H), 8.80 (s, |
| | | 1H), 8.79-8.76 (m, 2H), |
| | | 8.27-8.18 (m, 2H), 7.18- |
| | | 7.08 (m, 2H), 4.41 (s, 3H), |
| | | 4.09 (s, 3H), 3.94 (s, 3H), |
| | | 3.68 (d, J = 12.3 Hz, 2H), |
| | | 3.45-3.43 (m, 1H), 2.95 (t, |
| | | J = 12.0 Hz, 2H), 2.79 (s, |
| | | 1H), 2.22 (d, J = 12.3 Hz, |
| | | 2H), 1.81-1.69 (m, 2H), |
| | | 0.85-0.83 (m, 4H). |

### Example 19: Synthesis of Compound 140

### Synthesis of Intermediate A85

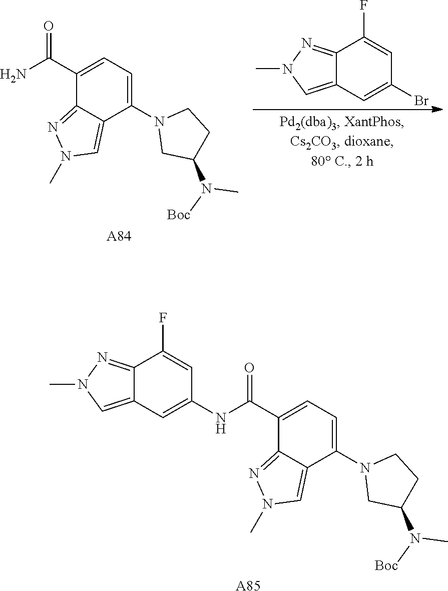

To a solution of tert-butyl N-[(3R)-1-(7-carbamoyl-2-methyl-indazol-4-yl)pyrrolidin-3-yl]-N-methyl-carbamate (A84, 140 mg, 374.9 μmol) and 5-bromo-7-fluoro-2-methyl-indazole (103 mg, 449.9 μmol) in dioxane (2 mL) were added Pd₂(dba)₃ (34 mg, 37.5 μmol), Xantphos (43 mg, 74.98 μmol) and Cs₂CO₃ (489 mg, 1.5 mmol), and the reaction was stirred for 2 h at 80° C. under N₂. The resulting mixture was concentrated under reduced pressure and purified by silica gel column chromatography (89% EtOAc in PE) to afford tert-butyl N-[(3R)-1-[7-[(7-fluoro-2-methyl-indazol-5-yl)carbamoyl]-2-methyl-indazol-4-yl]pyrrolidin-3-yl]-N-methyl-carbamate (A85, 100 mg, 191.7 μmol). LCMS (ES, m/z): 522 [M+H]⁺.

### Synthesis of Compound 140

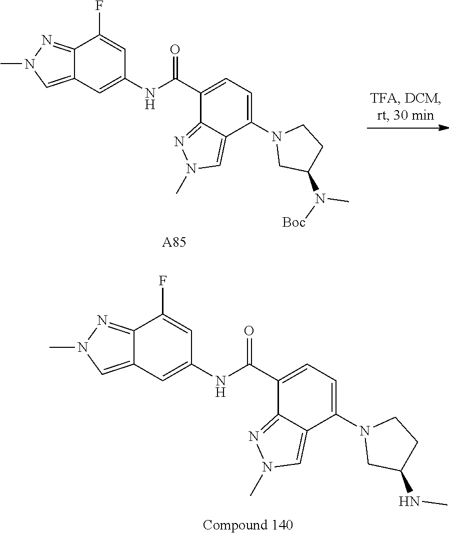

A solution of Intermediate A85 (90 mg, 172.6 μmol) and TFA (0.2 mL) in DCM (1 mL) was stirred for 30 min at room temperature. The mixture was neutralized to pH 7 with NH₃ (g) in MeOH, concentrated under reduced pressure, and purified by reversed-phase flash chromatography (Condition 3, Gradient 8) to afford N-(7-fluoro-2-methyl-indazol-5-yl)-2-methyl-4-[(3R)-3-(methylamino)pyrrolidin-1-yl]indazole-7-carboxamide (Compound 140, 11 mg, 26.1 μmol). LCMS (ES, m/z): 422 [M+H]⁺. ¹H NMR (300 MHz, DMSO-d₆) δ 11.13 (s, 1H), 8.83 (s, 1H), 8.40 (d, J=2.9 Hz, 1H), 8.02 (d, J=1.6 Hz, 1H), 7.94 (d, J=8.3 Hz, 1H), 7.43 (dd, J=13.4, 1.6 Hz, 1H), 6.01 (d, J=8.3 Hz, 1H), 4.28 (s, 3H), 4.19 (s, 3H), 3.85-3.57 (m, 3H), 3.47-3.37 (m, 2H), 2.35 (s, 3H), 2.17 (dt, J=12.6, 6.7 Hz, 1H), 1.98-1.88 (m, 1H).

### Example 20: Synthesis of Compound 143

### Synthesis of Intermediate A87

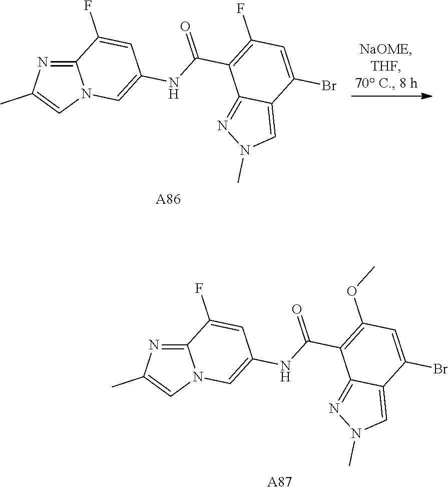 [SMILES: Cc1cn2cc(NC(=O)c3c(F)cc(Br)c4cn(C)nc34)cc(F)c2n1 ; "A86" ; COc1cc(Br)c2cn(C)nc2c1C(=O)Nc1cc(F)c2nc(C)cn2c1 ; "A87"]

To a solution of 4-bromo-6-fluoro-N-(8-fluoro-2-methyl-imidazo[1,2-a]pyridin-6-yl)-2-methyl-indazole-7-carboxamide (A86, 0.34 g, 809.1 μmol) in THF (3 mL) was added NaOMe ((30 wt. %) solution in MeOH; 219 mg, 4.05 mmol), and the reaction was stirred for 8 h at 70° C. The resulting mixture was allowed to cool to room temperature, quenched by the addition of water (20 mL), and extracted with EtOAc (3×30 mL). The combined organic layers were washed with brine (20 mL), dried over anhydrous Na₂SO₄, and filtered. The filtrate was concentrated under reduced pressure and purified by silica gel column chromatography (91% EtOAc in PE) to afford 4-bromo-N-(8-fluoro-2-methyl-imidazo[1,2-a]pyridin-6-yl)-6-methoxy-2-methyl-indazole-7-carboxamide (A87, 0.31 g, 717.2 μmol). LCMS (ES, m/z): 432 [M+H]⁺.

### Synthesis of Intermediate A88

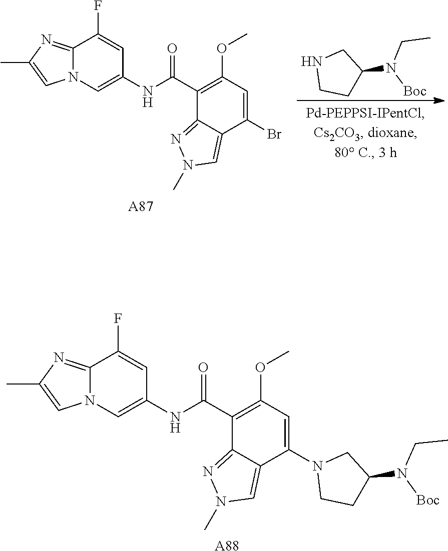

To a stirred solution of Intermediate A87 (0.35 g, 811.6 μmol) and tert-butyl N-ethyl-N-[(3S)-pyrrolidin-3-yl]carbamate (174 mg, 811.6 μmol) in dioxane (3 mL) were added Cs₂CO₃ (264 mg, 811.6 μmol) and Pd-PEPPSI-IPentCl (204 mg, 243.5 μmol), and the reaction was stirred for 3 h at 80° C. under N₂. The resulting mixture was allowed to cool to room temperature, concentrated under reduced pressure, and purified by silica gel column chromatography (9% MeOH in DCM) to afford tert-butyl N-ethyl-N-[(3S)-1-[7-[(8-fluoro-2-methyl-imidazo[1,2-a]pyridin-6-yl)carbamoyl]-6-methoxy-2-methyl-indazol-4-yl]-pyrrolidin-3-yl]carbamate (A88, 0.28 g, 495.0 μmol). LCMS (ES, m/z): 566 [M+H]⁺.

### Synthesis of Compound 143

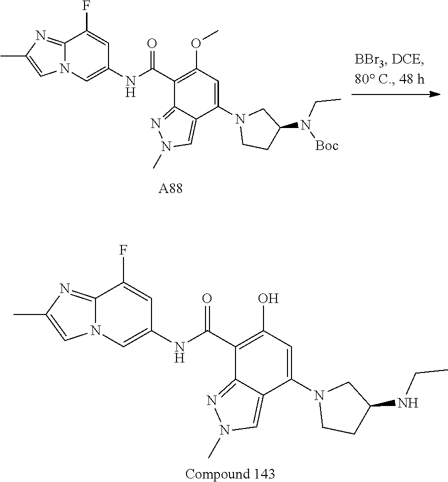

To a stirred solution of Intermediate A88 (0.18 g, 318.2 μmol) in DCE (3 mL) was added BBr₃ (796 mg, 3.18 mmol) in portions, and the reaction was stirred for 48 h at 80° C. under N₂. The resulting mixture was concentrated under reduced pressure, quenched by the addition of water (10 mL) at room temperature, then basified to pH 8 with saturated aq. NaHCO₃, and extracted with DCM (3×30 mL). The combined organic layers were washed with brine (30 mL), dried over anhydrous Na₂SO₄, and filtered. The filtrate was concentrated under reduced pressure and purified by reverse flash chromatography (Condition n 1, Gradient 2) to afford 4-[(3S)-3-(ethylamino)pyrrolidin-1-yl]-N-(8-fluoro-2-methyl-imidazo[1,2-a]pyridin-6-yl)-6-hydroxy-2-methyl-indazole-7-carboxamide (Compound 143, 53 mg, 117.4 μmol). LCMS (ES, m/z): 452 [M+H]⁺. ¹H NMR (300 MHz, DMSO-d₆) δ 13.49 (s, 1H), 11.01 (s, 1H), 9.03 (d, J=1.7 Hz, 1H), 8.66 (s, 1H), 7.89 (d, J=3.0 Hz, 1H), 7.30 (dd, J=12.3, 1.7 Hz, 1H), 5.49 (s, 1H), 4.17 (s, 3H), 3.83 (s, 1H), 3.73 (s, 1H), 3.60 (s, 2H), 2.63 (q, J=7.0 Hz, 2H), 2.35 (s, 3H), 2.15 (dd, J=12.2, 6.0 Hz, 1H), 1.90 (d, J=7.7 Hz, 1H), 1.05 (t, J=7.1 Hz, 3H).

An analogous method was followed to obtain the following compounds.

| Compound | Starting Material | Characterization |
|---|---|---|
| 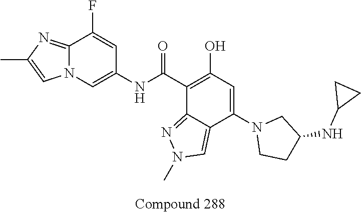 [SMILES: Cc1cn2cc(NC(=O)c3c(O)cc(N4CC[C@@H](NC5CC5)C4)c4cn(C)nc34)cc(F)c2n1] | 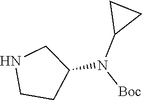 [SMILES: CC(C)(C)OC(=O)N(C1CC1)[C@@H]1CCNC1] | The residue was purified by reversed-phase flash chroma- tography (Condition 1, Gradient 1) to afford 4-[(3R)- 3-(cyclopropylamino)-pyr- rolidin-1-yl]-N-(8-fluoro-2- methyl-imidazo[1,2-a]pyridin- 6-yl)-6-hydroxy-2-methyl- indazole-7-carboxamide (Compound 288, 30 mg, 64.7 μmol, 25% yield) as a solid. LCMS (ES, m/z): 464 [M + H]⁺. ¹H NMR (400 MHz, |
| | | DMSO-d₆) δ 13.49 (s, 1H), |
| | | 11.01 (s, 1H), 9.03 (d, J = 1.6 |
| | | Hz, 1H), 8.66 (s, 1H), 7.89 (d, |
| | | J = 3.1 Hz, 1H), 7.30 (d, J = |
| | | 12.3 Hz, 1H), 5.48 (s, 1H), |
| | | 4.17 (s, 3H), 3.74 (s, 1H), |
| | | 3.55 (t, J = 14.1 Hz, 3H), 3.43 |
| | | (s, 1H), 2.35 (s, 3H), 2.21- |
| | | 2.10 (m, 2H), 2.01-1.92 (m, |
| | | 1H), 0.42 (d, J = 6.8 Hz, 2H), |
| | | 0.31-0.21 (m, 2H). |

### Example 21: Synthesis of Compound 146

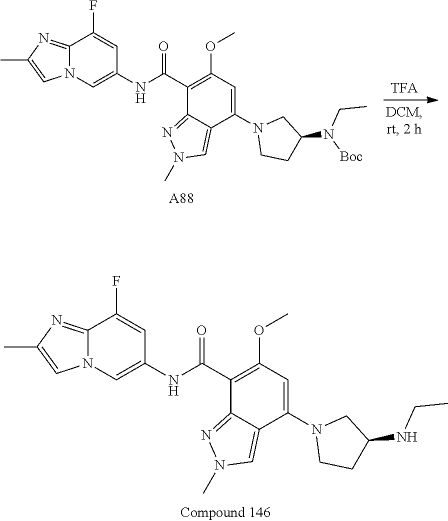

To a stirred solution of Intermediate A88 (0.12 g, 212.2 μmol) in DCM (3 mL) was added TFA (0.2 mL), and the reaction was stirred for 2 h at room temperature. The resulting mixture was concentrated under reduced pressure and purified by reverse flash chromatography (Condition 1, Gradient 3) to afford tert-butyl 4-[(3S)-3-(ethylamino)pyrrolidin-1-yl]-N-(8-fluoro-2-methyl-imidazo[1,2-a]pyridin-6-yl)-6-methoxy-2-methyl-indazole-7-carboxamide (Compound 146, 31 mg, 66.6 μmol). LCMS (ES, m/z): 466 [M+H]⁺. ¹H NMR (300 MHz, DMSO-d₆) δ 10.86 (s, 1H), 9.22 (s, 1H), 8.64 (s, 1H), 7.85 (s, 1H), 7.20 (d, J=12.6 Hz, 1H), 5.69 (s, 1H), 4.14 (s, 3H), 3.87 (s, 3H), 3.75 (dd, J=16.5, 8.0 Hz, 2H), 3.63 (s, 1H), 3.44 (s, 2H), 2.63 (q, J=6.9 Hz, 2H), 2.34 (s, 3H), 2.16 (s, 1H), 1.89 (s, 2H), 1.05 (t, J=7.1 Hz, 3H).

An analogous method was followed to obtain the following compound.

| Compound | Starting Material | Characterization |
|---|---|---|
| 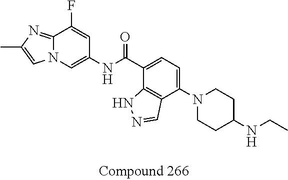 [SMILES: CCNC1CCN(c2ccc(C(=O)Nc3cc(F)c4nc(C)cn4c3)c3[nH]ncc23)CC1] | 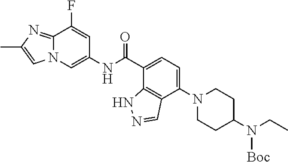 [SMILES: CCN(C(=O)OC(C)(C)C)C1CCN(c2ccc(C(=O)Nc3cc(F)c4nc(C)cn4c3)c3[nH]ncc23)CC1] | Modification: The reaction was ran for 30 min. The residue was purified by reversed-phase flash chromatography (Condition 3, Gradient 13) to afford 4- [4-(ethylamino)piperidin-1- yl]-N-(8-fluoro-2-methyl- imidazo[1,2-a]pyridin-6-yl)- 1H-indazole-7-carboxamide (Compound 266, 9.7 mg, 13% yield) as a solid. LCMS (ES, m/z): 436 [M + H]⁺. ¹H NMR (400 MHz, DMSO- d₆) δ 13.18-12.74 (m, 1H), 10.16 (s, 1H), 9.06 (d, J = 1.4 Hz, 1H), 8.21 (s, 1H), 7.97 (d, J = 8.3 Hz, 1H), 7.90 (s, 1H), 7.39 (d, J = 12.7 Hz, 1H), 6.55 (d, J = 8.3 Hz, 1H), 3.95 (d, J = 12.6 Hz, 2H), 3.11 (t, J = 11.7 Hz, 2H), 2.73-2.64 (m, 1H), 2.62 (q, J = 6.9 Hz, 2H), 2.35 (s, 3H), 1.97 (d, J = 12.8 Hz, 2H), 1.52- 1.42 (m, 2H), 1.04 (t, J = 7.0 Hz, 3H). |

### Example 22: Synthesis of Compound 128

### Synthesis of Intermediate A90

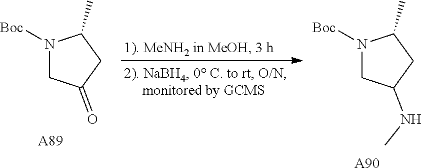

To a stirred solution of tert-butyl(2R)-2-methyl-4-oxo-pyrrolidine-1-carboxylate (A89, 1 g, 5.02 mmol) in MeOH (20 mL) was added MeNH₂ in MeOH (10 mL), and the resulting mixture was stirred for 3 h at room temperature. To the above mixture was added NaBH₄ (285 mg, 7.53 mmol) in portions at 0° C., stirred overnight at room temperature, then quenched with water (20 mL) and concentrated under reduced pressure. The residue was diluted with aqueous citric acid (20%, 30 mL) and the mixture was extracted with DCM (3×100 mL). The aqueous layer was basified to pH 8-9 with solid Na₂CO₃ and later extracted with DCM (4×100 mL). The combined organic layers were dried and concentrated to afford tert-butyl(2R)-2-methyl-4-(methylamino)pyrrolidine-1-carboxylate (A90, 1.3 g, crude). LCMS (ES, m/z): 215 [M+H]⁺.

### Synthesis of Intermediate A91

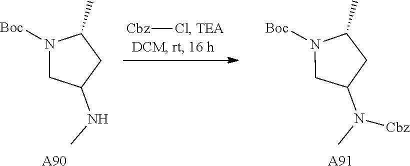

To a stirred mixture of Intermediate A90 (1.3 g, 6.07 mmol) and TEA (1.84 g, 18.2 mmol, 2.5 mL) in DCM (26 mL) was added benzyl carbonochloridate (1.09 g, 6.37 mmol, 898 μL) dropwise at 0° C., and the reaction was stirred for 16 h at room temperature. The resulting mixture was diluted with DCM (100 mL), washed with citric acid solution (100 mL), saturated NaHCO₃ solution (100 mL), brine (100 mL) and dried over anhydrous Na₂SO₄, and filtered. The filtrate was concentrated under reduced pressure and purified by silica gel column chromatography (9% THF in PE) to afford tert-butyl(2R)-4-[benzyloxycarbonyl(methyl)amino]-2-methyl-pyrrolidine-1-carboxylate (A91, 920 mg, crude). LCMS (ES, m/z): 349 [M+H]⁺.

### Synthesis of Intermediate A92

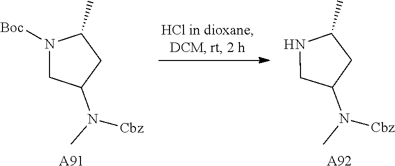

To a stirred solution of Intermediate A91 (350 mg, 1.00 mmol) in dioxane (2 mL) was added HCl in dioxane (2 mL), and the reaction was stirred for 2 h at room temperature. The resulting mixture was concentrated under reduced pressure to afford benzyl N-methyl-N-[(5R)-5-methylpyrrolidin-3-yl]carbamate hydrochloride (A92, 352 mg, crude). LCMS (ES, m/z): 249 [M+H]⁺.

### Synthesis of Intermediate A93

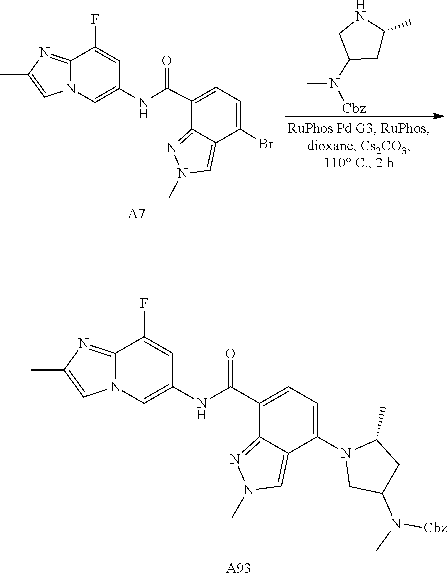

To a stirred mixture of Intermediate A7 (200 mg, 515.2 μmol) and Intermediate A92 (147 mg, 515.2 μmol) in dioxane (4 mL) were added Cs₂CO₃ (504 mg, 1.55 mmol), Ruphos (48 mg, 103.0 μmol) and RuPhos Pd G3 (43 mg, 51.52 μmol), and the reaction was stirred for 2 h at 110° C. under N₂. The resulting mixture was concentrated under reduced pressure and purified by silica gel column chromatography (9% MeOH in DCM) to afford benzyl N-[(5R)-1-[7-[(8-fluoro-2-methylimidazo[1,2-a]pyridin-6-yl)carbamoyl]-2-methyl-indazol-4-yl]-5-methyl-pyrrolidin-3-yl]-N-methyl-carbamate (A93, 158 mg, 277 μmol). LCMS (ES, m/z): 536 [M+H]⁺.

### Synthesis of Compound 128

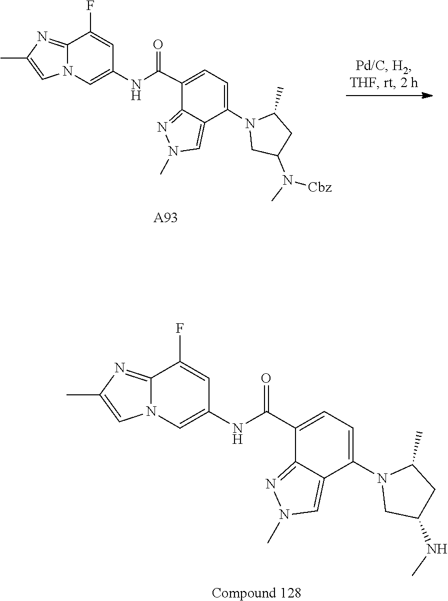 [SMILES: Cc1cn2cc(NC(=O)c3ccc(N4CC(N(C)C(=O)OCc5ccccc5)C[C@H]4C)c4cn(C)nc34)cc(F)c2n1 ; "A93" ; CN[C@H]1C[C@@H](C)N(c2ccc(C(=O)Nc3cc(F)c4nc(C)cn4c3)c3nn(C)cc23)C1 ; "Compound 128"]

To a solution of Intermediate A93 (158 mg, 277.4 μmol) in THF (20 mL) was added Pd/C (30 mg, 28.19 μmol, 10% purity), and the mixture was hydrogenated at room temperature for 2 h under hydrogen atmosphere using a hydrogen balloon. The resulting mixture was filtered through a Celite pad, concentrated under reduced pressure and purified by Prep-HPLC (Condition 5, Gradient 5) afford the product (50 mg). The product was purified by Prep-Chiral-HPLC (Condition 1, Gradient 1) to afford N-(8-fluoro-2-methyl-imidazo[1,2-a]-pyridin-6-yl)-2-methyl-4-[(2R,4S)-2-methyl-4-(methylamino)pyrrolidin-1-yl]indazole-7-carboxamide (Compound 128, 34.4 mg, 78.99 μmol). Absolute stereochemistry was assigned arbitrarily. LCMS (ES, m/z): 436 [M+H]⁺. ¹H NMR (400 MHz, CDCl₃) δ 10.98 (s, 1H), 9.26 (s, 1H), 8.21 (d, J=8.2 Hz, 1H), 8.11 (s, 1H), 7.42 (d, J=3.0 Hz, 1H), 6.86 (d, J=11.2 Hz, 1H), 6.19 (d, J=8.4 Hz, 1H), 4.26 (s, 4H), 4.09-4.01 (m, 1H), 3.72 (s, 1H), 3.42 (t, J=7.0 Hz, 1H), 2.59 (s, 4H), 2.49 (s, 3H), 1.79-1.70 (m, 1H), 1.44 (d, J=6.0 Hz, 3H).

An analogous method was followed to obtain the following compounds.

| Compound | Characterization |
|---|---|
| 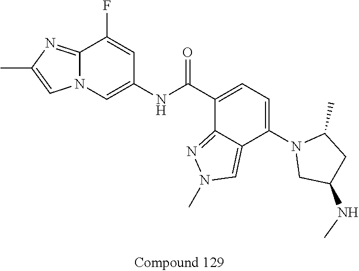 [SMILES: CN[C@@H]1C[C@@H](C)N(c2ccc(C(=O)Nc3cc(F)c4nc(C)cn4c3)c3nn(C)cc23)C1] | The product was purified by Prep-Chiral-HPLC (Condition 1, Gradient 1) to afford N-(8-fluoro- 2-methyl-imidazo[1,2-a]pyridin-6-yl)-2-methyl- 4-[(2R,4R)-2-methyl-4-(methylamino)pyrrol- idin-1-yl]indazole-7-carboxamide (Compound 129, 3.4 mg, 7.81 μmol). LCMS (ES, m/z): 436 [M + H]⁺. ¹H NMR (400 MHz, CDCl₃) δ 10.91 (s, 1H), 9.12 (s, 1H), 8.11 (s, 1H), 8.00 (s, 1H), 7.37 (s, 1H), 6.69 (s, 1H), 6.03 (s, 1H), 4.29 (d, J = 19.5 Hz, 6H), 4.11-4.03 (m, 1H), 3.73 (s, 2H), 2.65 (s, 3H), 2.51 (s, 4H), 2.16- 2.08 (m, 1H), 1.79-1.70 (m, 1H), 1.52-1.39 (m, 2H), 1.34-1.26 (m, 4H). |

### Example 23: Synthesis of Compound 149

### Synthesis of Intermediate A95

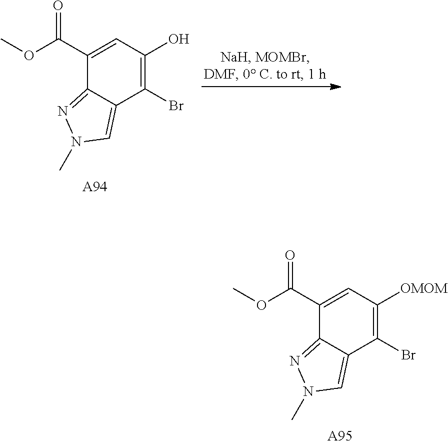

A solution of methyl 4-bromo-5-hydroxy-2-methyl-indazole-7-carboxylate (A94, 400 mg, 1.40 mmol) in DMF (8 mL) was treated with NaH (67.3 mg, 2.81 mmol) for 1 h at 0° C. under N₂ followed by the addition of MOMBr (347.8 mg, 2.81 mmol) dropwise at 0° C. The reaction was quenched by the addition of saturated NH₄Cl (aq.) (50 mL) at 0° C. and then extracted with EtOAc (2×50 mL). The combined organic layers were washed with water (2×100 mL), brine (100 mL), dried over anhydrous Na₂SO₄, filtered, and concentrated under reduced pressure to afford methyl 4-bromo-5-(methoxymethoxy)-2-methyl-indazole-7-carboxylate (A95, 420 mg, 1.28 mmol). LCMS (ES, m/z): 329 [M+H]⁺.

### Synthesis of Intermediate A96

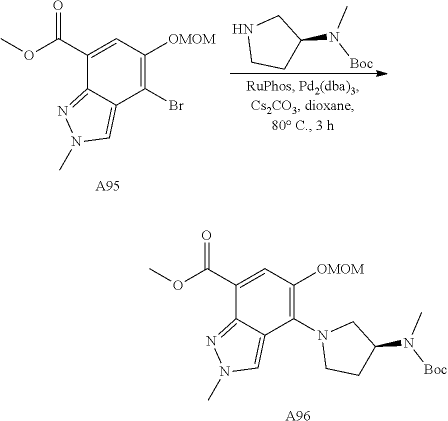

To a solution of Intermediate A95 (400 mg, 1.22 mmol) and tert-butyl N-methyl-N-[(3S)-pyrrolidin-3-yl]carbamate (292 mg, 1.46 mmol) in dioxane (10 mL) were added Cs₂CO₃ (792 mg, 2.43 mmol), Pd₂(dba)₃ (111 mg, 121.5 μmol), and RuPhos (113.4 mg, 243 μmol), and the reaction was stirred for 3 h at 80° C. under N₂. The resulting mixture was concentrated under reduced pressure and purified by silica gel column chromatography (50% EtOAc in PE) to afford methyl 4-[(3S)-3-[tert-butoxycarbonyl(methyl)amino]pyrrolidin-1-yl]-5-(methoxymethoxy)-2-methyl-indazole-7-carboxylate (A96, 440 mg, 0.98 mmol). LCMS (ES, m/z): 449 [M+H]⁻.

### Synthesis of Intermediate A97

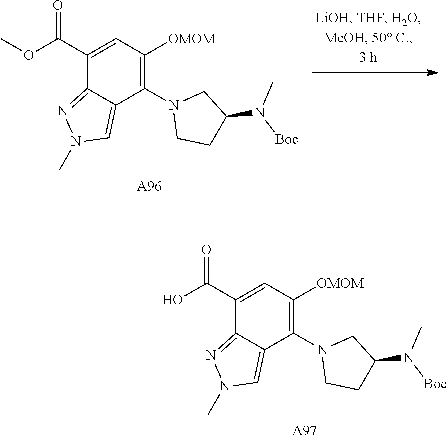

To a stirred mixture of Intermediate A96 (440.0 mg, 981.0 μmol) in THF (3 mL), MeOH (3 mL), and H₂O (3 mL) was added LiOH (164.4 mg, 6.87 mmol), and the reaction was stirred for 3 h at 50° C. The resulting mixture was diluted with water (10 mL), acidified to pH 6 with HCl (2 M), and extracted with EtOAc (2×10 mL). The combined organic layers were washed with brine (20 mL), dried over anhydrous Na₂SO₄, filtered, and concentrated under reduced pressure to afford 4-[(3S)-3-[tert-butoxycarbonyl-(methyl)amino]pyrrolidin-1-yl]-5-(methoxymethoxy)-2-methyl-indazole-7-carboxylic acid (A97, 380 mg, 874.6 μmol). LCMS (ES, m/z): 435 [M+H]⁺.

### Synthesis of Intermediate A98

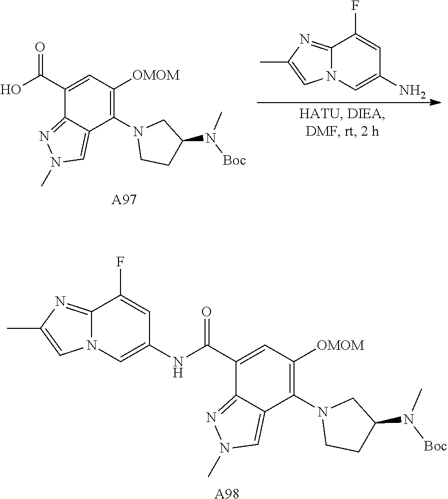

To a stirred mixture of Intermediate A97 (350.0 mg, 805.6 μmol) and 8-fluoro-2-methyl-imidazo[1,2-a]pyridin-6-amine (146.3 mg, 886.1 μmol) in DMF (7 mL) were added DIEA (416.4 mg, 3.22 mmol, 561.2 μL) and HATU (612.6 mg, 1.61 mmol), and the reaction was stirred for 2 h at room temperature. The resulting mixture was quenched with water (20 mL) and extracted with EtOAc (2×20 mL). The combined organic layers were washed with water (2×20 mL), brine (20 mL), dried over anhydrous Na₂SO₄, and filtered. The filtrate was concentrated under reduced pressure and purified by silica gel column chromatography (67% EtOAc in PE) to afford tert-butyl N-[(3S)-1-[7-[(8-fluoro-2-methyl-imidazo[1,2-a]pyridin-6-yl)carbamoyl]-5-(methoxymethoxy)-2-methyl-indazol-4-yl]pyrrolidin-3-yl]-N-methyl-carbamate (A98, 240 mg, 412.6 μmol). LCMS (ES, m/z): 582 [M+H]⁺.

### Synthesis of Compound 149

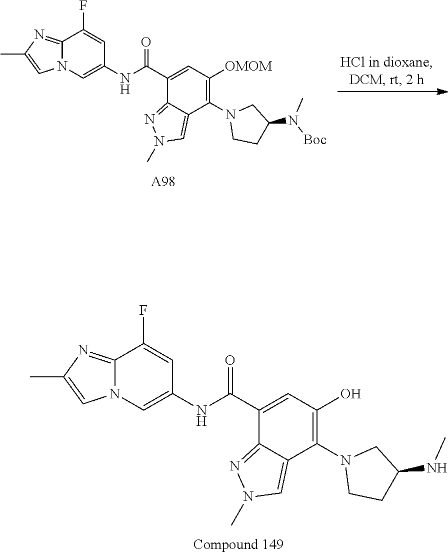

To a stirred mixture of Intermediate A98 (50 mg, 85.96 μmol) in DCM (1 mL) was added HCl (4 M in 1,4-dioxane) (0.5 mL), and the reaction was stirred for 2 h at room temperature. The resulting mixture was concentrated under reduced pressure and purified by trituration with Et₂O (5 mL) to afford N-(8-fluoro-2-methyl-imidazo[1,2-a]pyridin-6-yl)-5-hydroxy-2-methyl-4-[(3S)-3-(methylamino)pyrrolidin-1-yl]-indazole-7-carboxamide (Compound 149, 10 mg, 22.86 μmol). LCMS (ES, m/z): 438 [M+H]⁺. ¹H NMR (300 MHz, DMSO-d₆) δ 11.38 (s, 1H), 9.58 (d, J=1.5 Hz, 1H), 9.23 (s, 2H), 8.89 (s, 1H), 8.79 (s, 1H), 8.27 (d, J=1.8 Hz, 1H), 8.07 (d, J=11.8 Hz, 1H), 7.81 (s, 1H), 4.27 (s, 3H), 4.04 (t, J=8.5 Hz, 3H), 3.80-3.79 (m, 2H), 2.63 (t, J=5.3 Hz, 3H), 2.51 (s, 3H), 2.33 (q, J=6.6 Hz, 1H), 2.17 (dd, J=12.5, 6.4 Hz, 1H).

### Example 24: Synthesis of Compound 179

### Synthesis of Intermediate A100

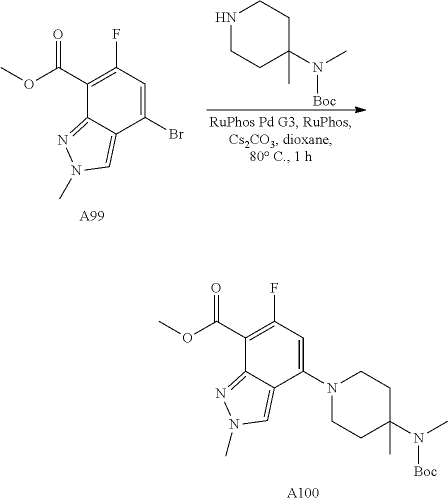

To a solution of methyl 4-bromo-6-fluoro-2-methyl-indazole-7-carboxylate (A99, 0.5 g, 1.74 mmol) and tert-butyl N-methyl-N-(4-methyl-4-piperidyl)carbamate (397 mg, 1.74 mmol) in dioxane (9 mL) were added Cs₂CO₃ (1.7 g, 5.22 mmol), RuPhos Pd G3 (146 mg, 174.2 μmol) and Ruphos (163 mg, 348.3 μmol), and the reaction was stirred for 1 h at 80° C. under N₂. The resulting mixture was concentrated under reduced pressure and purified by silica gel column chromatography (89% EtOAc in PE) to afford methyl 4-[4-[tert-butoxycarbonyl(methyl)amino]-4-methyl-1-piperidyl]-6-fluoro-2-methyl-indazole-7-carboxylate (A100, 0.7 g, 1.61 mmol). LCMS (ES, m/z): 435 [M+H]⁺.

### Synthesis of Intermediate A101

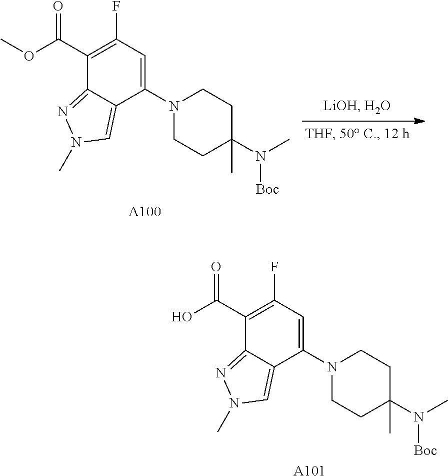

To a stirred solution of Intermediate A100 (0.5 g, 1.15 mmol) in THF (3 mL) and H₂O (3 mL) was added LiOH·H₂O (290 mg, 6.90 mmol), and the reaction was stirred for 12 h at 50° C. The resulting mixture was acidified to pH 2 with 1 M HCl (aq), and the precipitated solids were collected by filtration and washed with H₂O (2×2 mL). The crude product (4-[4-[tert-butoxy-carbonyl(methyl)amino]-4-methyl-1-piperidyl]-6-fluoro-2-methyl-indazole-7-carboxylic acid (A101, 0.4 g, 951.3 μmol) was used in the next step directly without further purification. LCMS (ES, m/z): 421 [M+H]⁺.

### Synthesis of Intermediate A102

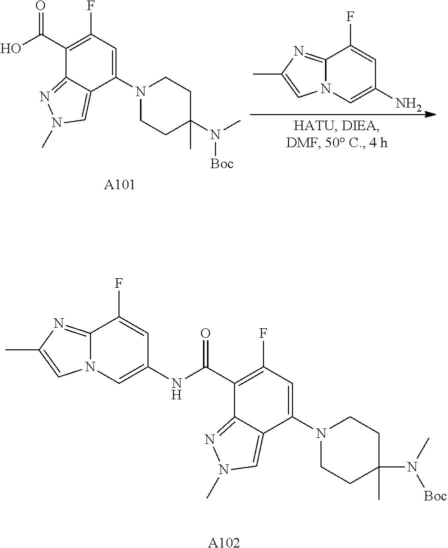 [SMILES: CN(C(=O)OC(C)(C)C)C1(C)CCN(c2cc(F)c(C(=O)O)c3nn(C)cc23)CC1 ; Cc1cn2cc(N)cc(F)c2n1 ; "A101" ; Cc1cn2cc(NC(=O)c3c(F)cc(N4CCC(C)(N(C)C(=O)OC(C)(C)C)CC4)c4cn(C)nc34)cc(F)c2n1 ; "A102"]

To a stirred solution of Intermediate A101 (0.1 g, 237.8 μmol) and HATU (136 mg, 356.7 μmol) in DMF (2 mL) were added DIEA (92 mg, 713.5 μmol, 124.3 μL) and 8-fluoro-2-methyl-imidazo[1,2-a]pyridin-6-amine (43 mg, 261.6 μmol) at room temperature. The resulting mixture was stirred for 4 h at 50° C. and then purified by reversed-phase flash chromatography (Condition 3, Gradient 6) to afford tert-butyl N-[1-[6-fluoro-7-[(8-fluoro-2-methyl-imidazo[1,2-a]pyridin-6-yl)carbamoyl]-2-methyl-indazol-4-yl]-4-methyl-4-piperidyl]-N-methyl-carbamate (A102, 0.11 g, 193.8 μmol). LCMS (ES, m/z): 568 [M+H]⁺.

### Synthesis of Compound 179

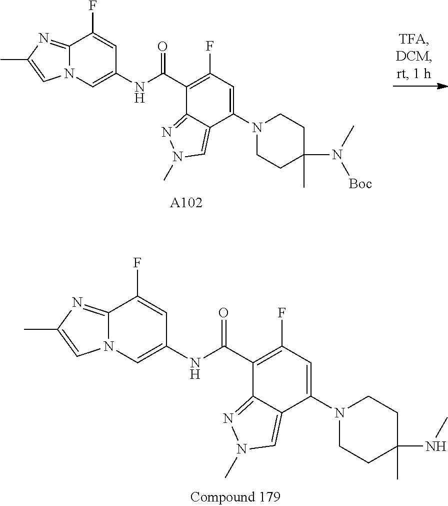

To a stirred solution of Intermediate A102 (0.1 g, 176.2 μmol) in DCM (2 mL) was added TFA (0.2 mL) dropwise, and the reaction was stirred for 1 h at room temperature. The resulting mixture was basified to pH 8 with NH₃ (g) in MeOH, concentrated under reduced pressure, and purified by reversed-phase flash chromatography (Condition 3, Gradient 9) to afford 6-fluoro-N-(8-fluoro-2-methyl-imidazo[1,2-a]pyridin-6-yl)-2-methyl-4-[4-methyl-4-(methylamino)-1-piperidyl]indazole-7-carboxamide (Compound 179, 0.04 g, 85.56 μmol). LCMS (ES, m/z): 468 [M+H]⁺. ¹H NMR (300 MHz, DMSO-d₆) δ 10.93 (s, 1H), 9.19 (d, J=1.7 Hz, 1H), 8.75 (s, 1H), 7.89 (d, J=3.1 Hz, 1H), 7.24 (dd, J=12.5, 1.6 Hz, 1H), 6.20 (d, J=15.6 Hz, 1H), 4.20 (s, 3H), 3.49 (t, J=5.3 Hz, 4H), 2.35 (s, 3H), 2.22 (s, 3H), 1.67 (d, J=13.5 Hz, 2H), 1.56 (dt, J=13.5, 6.6 Hz, 2H), 1.08 (s, 3H).

An analogous method was followed to obtain the following compounds.

| Compounds | Starting Material | Characterization |
|---|---|---|
| 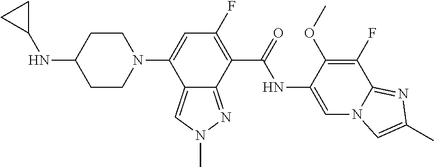 [SMILES: COc1c(NC(=O)c2c(F)cc(N3CCC(NC4CC4)CC3)c3cn(C)nc23)cn2cc(C)nc2c1F] Compound 207 | 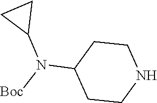 [SMILES: CC(C)(C)OC(=O)N(C1CCNCC1)C1CC1] 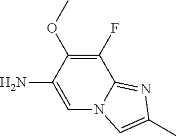 | The first step was ran for 6 h. The reaction mixture was concentrated and then lyophilized for 1 day to afford 4-[4-(cyclopropylamino)-1- piperidyl]-6-fluoro-N-(8- fluoro-7-methoxy-2-methyl- imidazo[1,2-a]pyridin-6-yl)- 2-methyl-indazole-7- carboxamide (Compound 207, 19 mg, 92%, as CF₃CO₂H salt) as a solid. LCMS (ES, m/z): 510.2 [M + H]⁺. ¹H NMR (400 MHz, Methanol-d₄) δ ppm 9.74 (d, J = 0.88 Hz, 1H), |
| | | 8.62 (s, 1H), 7.90 (s, 1H), 6.37 |
| | | (d, J = 15.76 Hz, 1H), 4.54 (d, |
| | | J = 4.38 Hz, 3H), 4.32 (s, 3H), |
| | | 4.20 (br d, J = 13.13 Hz, 2H), |
| | | 3.60 (br t, J = 4.19 Hz, 1H), |
| | | 3.20 (br t, J = 12.07 Hz, 2H), |
| | | 2.85 (dt, J = 7.35, 3.52 Hz, 1H), |
| | | 2.52 (s, 3H), 2.35 (br d, J = |
| | | 9.88 Hz, 2H), 1.80 - 1.89 (m, |
| | | 2H), 1.00 (br d, J = 7.00 Hz, |
| | | 2H), 0.88-0.93 (m, 2H). ¹⁹F |
| | | NMR (376 MHz, Methanol-d₄) |
| | | δ ppm −77.02 (s, 1.7F), |
| | | −106.61 (s, 1F), −160.02 |
| | | (s, 1F). |
| 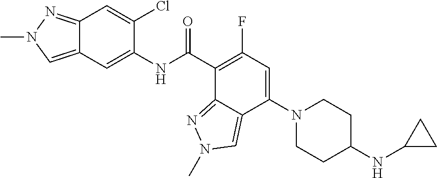 [SMILES: Cn1cc2cc(NC(=O)c3c(F)cc(N4CCC(NC5CC5)CC4)c4cn(C)nc34)c(Cl)cc2n1] Compound 439 | 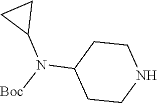 [SMILES: CC(C)(C)OC(=O)N(C1CCNCC1)C1CC1] 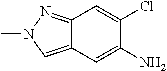 C157 | The crude product was purified by Prep-HPLC (Condition 6, Gradient 7) to afford N-(6-chloro-2-methyl- indazol-5-yl)-4-[4- (cyclopropylamino)-1- piperidyl]-6-fluoro-2-methyl- indazole-7-carboxamide (Compound 439, 23.5 mg, 47.4 μmol, 40% yield) as a solid. LCMS (ES, m/z): 496 [M + H]⁺. ¹H NMR (400 MHz, |
| | | DMSO-d₆) δ 10.80 (s, 1H), |
| | | 8.80 (s, 1H), 8.44 (s, 1H), 7.66 |
| | | (d, J = 8.8 Hz, 1H), 7.13 (d, J = |
| | | 8.8 Hz, 1H), 6.24 (d, J = 15.9 Hz, |
| | | 1H), 4.16 (d, J = 5.7 Hz, |
| | | 6H), 3.95-3.86 (m, 2H), 3.12 |
| | | (td, J = 12.9, 12.1, 2.7 Hz, 2H), |
| | | 2.81 (dt, J = 9.9, 5.5 Hz, 1H), |
| | | 2.13 (tt, J = 6.7, 3.5 Hz, 1H), |
| | | 2.04-1.96 (m, 2H), 1.47 (q, J = |
| | | 11.4, 9.5 Hz, 2H), 0.45-0.36 |
| | | (m, 2H), 0.28-0.20 (m, 2H). |
| 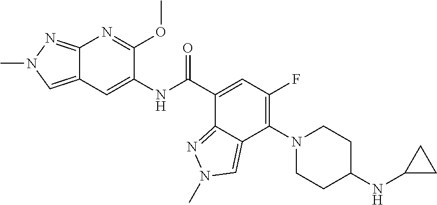 [SMILES: COc1nc2nn(C)cc2cc1NC(=O)c1cc(F)c(N2CCC(NC3CC3)CC2)c2cn(C)nc12] Compound 599 | 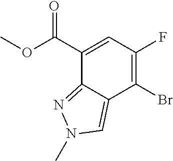 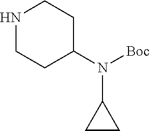 [SMILES: CC(C)(C)OC(=O)N(C1CCNCC1)C1CC1] 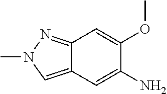 | Modifications: Step 1 was ran using Pd₂(dba)₃ and RuPhos, for 3 h. Step 2 was also ran in MeOH for 3 h at room temperature. Step 3 was ran for 2 h at room temperature. The residue was purified by reversed-phase flash chromatography (Condition 3, Gradient 5a) to afford 4-[4- (cyclopropylamino)-1- piperidyl]-5-fluoro-N-(6- methoxy-2-methyl-indazol-5- yl)-2-methyl-indazole-7- carboxamide (Compound 599, 0.05 g, 101.7 μmol). LCMS (ES, m/z): 492 [M + H]⁺. ¹H NMR (300 MHz, DMSO-d₆) δ 11.61 (s, 1H), 8.82 (s, 1H), 8.78 (s, 1H), 8.20 (s, 1H), 7.82 (d, J = 14.6 Hz, 1H), 7.08 (s, 1H), 4.30 (s, 3H), 4.09 (s, 3H), 4.06 (s, 3H), 3.74 (d, J = 12.3 Hz, 2H), 3.23 (t, J = |
| | | 11.5 Hz, 2H), 2.29 (s, 1H), |
| | | 2.19-2.10 (m, 1H), 2.00 (d, J = |
| | | 12.3 Hz, 2H), 1.49 (q, J = |
| | | 10.8 Hz, 2H), 0.40 (dt, J = 6.1, |
| | | 2.9 Hz, 2H), 0.29-0.21 (m, 2H). |

### Example 25: Synthesis of Compound 180

### Synthesis of Intermediate A103

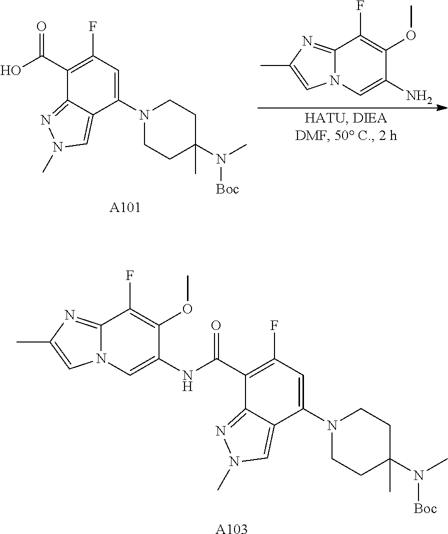

To a stirred solution of Intermediate A101 (0.1 g, 237.8 μmol) and HATU (136 mg, 356.7 μmol) in DMF (2 mL) were added DIEA (92 mg, 713.5 μmol) and 8-fluoro-7-methoxy-2-methyl-imidazo[1,2-a]pyridin-6-amine (46 mg, 237.8 μmol) at room temperature. The resulting mixture was stirred for 2 h at 50° C. and then purified by reversed-phase flash chromatography (Condition 3, Gradient 6) to afford tert-butyl N-[1-[6-fluoro-7-[(8-fluoro-7-methoxy-2-methyl-imidazo[1,2-a]pyridin-6-yl)carbamoyl]-2-methyl-indazol-4-yl]-4-methyl-4-piperidyl]-N-methyl-carbamate (A103, 0.09 g, 150.6 μmol). LCMS (ES, m/z): 598 [M+H]⁺.

### Synthesis of Compound 180

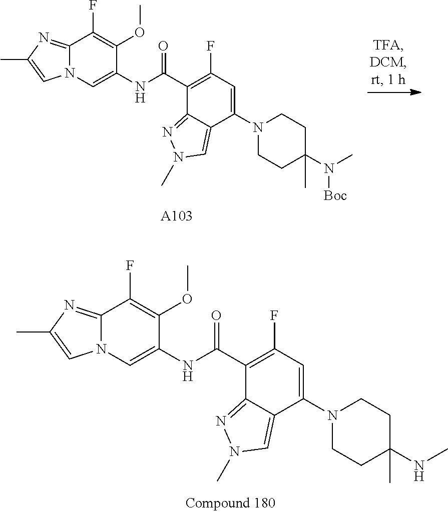

To a stirred solution of Intermediate A103 (0.09 g, 150.6 μmol) in DCM (2 mL) was added TFA (0.2 mL) dropwise, and the reaction was stirred for 1 h at room temperature. The resulting mixture was basified to pH 8 with NH₃ (g) in MeOH, concentrated under reduced pressure and purified by reversed-phase flash chromatography (Condition 3, Gradient 9) to afford 6-fluoro-N-(8-fluoro-7-methoxy-2-methyl-imidazo[1,2-a]pyridin-6-yl)-2-methyl-4-[4-methyl-4-(methylamino)-1-piperidyl]indazole-7-carboxamide (Compound 180, 37 mg, 74.4 μmol). LCMS (ES, m/z): 498 [M+H]⁺. ¹H NMR (300 MHz, DMSO-d₆) δ 11.56 (s, 1H), 9.46 (d, J=1.3 Hz, 1H), 8.83 (s, 1H), 7.76 (d, J=3.0 Hz, 1H), 6.21 (d, J=16.4 Hz, 1H), 4.26-4.15 (m, 6H), 3.55 (t, J=5.4 Hz, 4H), 2.31 (s, 3H), 2.23 (s, 3H), 1.73-1.51 (m, 4H), 1.09 (s, 3H).

An analogous method was followed to obtain the following compounds.

| Compound | Starting Material | Characterization |
|---|---|---|
| 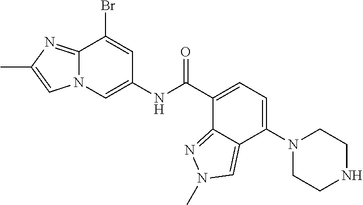 [SMILES: Cc1cn2cc(NC(=O)c3ccc(N4CCNCC4)c4cn(C)nc34)cc(Br)c2n1] Compound 265 | 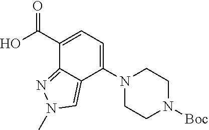 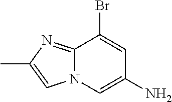 C81 | Modifications: Step 1 was ran at room temperature. Step 2 was ran for 2 h. The residue was purified by reversed-phase flash chromatography (Condition 3, Gradient 7) to afford N-(8- bromo-2-methyl-imidazo[1,2- a]-pyridin-6-yl)-2-methyl-4- piperazin-1-yl-indazole-7- carboxamide (Compound 265, 26.7 mg, 57 μmol, 41% yield) as a solid. LCMS (ES, m/z): 468 [M + H]⁺. ¹H NMR (300 MHz, DMSO-d₆) δ 11.04 (s, 1H), 9.36 (d, J = 1.8 Hz, |
| | | 1H), 8.80 (s, 1H), 7.98 (d, |
| | | J = 8.1 Hz, 1H), 7.92 (d, J = |
| | | 1.0 Hz, 1H), 7.71 (d, J = |
| | | 1.8 Hz, 1H), 6.49 (d, J = |
| | | 8.2 Hz, 1H), 4.30 (s, 3H), |
| | | 3.35 (d, J = 3.5 Hz, 4H), |
| | | 2.91 (dd, J = 6.1, 3.8 Hz, |
| | | 4H), 2.35 (s, 3H). |
| 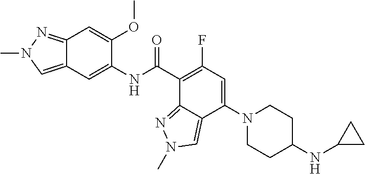 [SMILES: COc1cc2nn(C)cc2cc1NC(=O)c1c(F)cc(N2CCC(NC3CC3)CC2)c2cn(C)nc12] Compound 396 | 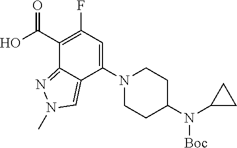 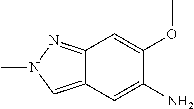 | Modification: Step 1 was ran at room temperature for 5 h. The mixture was purified by reversed-phase flash chromatography (Condition 3, Gradient 10) to afford 4-[4- (cyclopropylamino)-1- piperidyl]-6-fluoro-N-(6- methoxy-2-methyl-indazol-5- yl)-2-methyl-indazole-7- carboxamide (Compound 396, 33.1 mg, 67.3 μmol, 28% yield) as a solid. LCMS (ES, m/z): 492 [M + H]⁺. ¹H NMR (400 MHz, DMSO-d₆) δ 11.59 (s, 1H), 8.78 (s, 2H), 8.17 (s, 1H), 7.05 (s, 1H), 6.21 (d, J = |
| | | 16.1 Hz, 1H), 4.24 (s, 3H), 4.08 |
| | | (s, 3H), 4.03 (s, 3H), 3.89 (d, |
| | | J = 13.0 Hz, 2H), 3.10 (t, J = |
| | | 11.6 Hz, 2H), 2.80 (dt, J = |
| | | 10.0, 5.6 Hz, 1H), 2.12 (tt, J = |
| | | 6.7, 3.6 Hz, 1H), 1.99 (d, J = |
| | | 12.4 Hz, 2H), 1.45 (q, J = 10.7, |
| | | 9.8 Hz, 2H), 0.40 (dt, J = 6.2, |
| | | 3.1 Hz, 2H), 0.27-0.19 (m, 2H). |
| 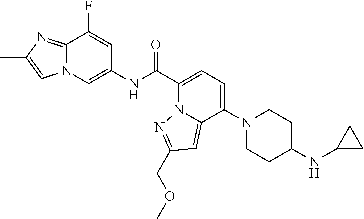 [SMILES: COCc1cc2c(N3CCC(NC4CC4)CC3)ccc(C(=O)Nc3cc(F)c4nc(C)cn4c3)n2n1] Compound 620 | 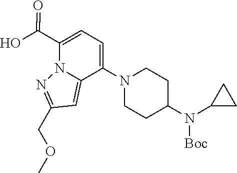 B157 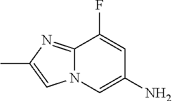 | Modification: Step 1 was ran at room temperature for 3 h. Step 2 was ran for 1 h at room temperature. The resulting mixture purified by reversed-phase flash chromatography (Condition 3, Gradient 8) to afford 4-[4- (cyclopropylamino)-1- piperidyl]-N-(8-fluoro-2- methyl-imidazo[1,2-a]pyridin- 6-yl)-2-(methoxymethyl)- pyrazolo[1,5-a]pyridine-7- carboxamide (Compound 620, 0.05 g, 101.7 μmol, 46% yield) as a solid. LCMS (ES, m/z): 492 [M + H]⁺. ¹H NMR |
| | | (400 MHz, DMSO-d₆) δ 12.49 |
| | | (s, 1H), 9.19 (d, J = 1.7 Hz, |
| | | 1H), 7.95-7.89 (m, 1H), 7.78 (d, |
| | | J = 8.1 Hz, 1H), 7.33 (dd, J = |
| | | 12.2, 1.7 Hz, 1H), 6.80 (s, 1H), |
| | | 6.75 (d, J = 8.2 Hz, 1H), 4.77 |
| | | (s, 2H), 3.71 (d, J = 12.5 Hz, |
| | | 2H), 3.42 (s, 3H), 2.95 (t, J = |
| | | 11.1 Hz, 2H), 2.76 (dt, J = 9.9, |
| | | 5.7 Hz, 1H), 2.38-2.33 (m, 3H), |
| | | 2.26 (s, 1H), 2.11 (tq, J = 8.5, |
| | | 4.9, 4.3 Hz, 1H), 2.01 (d, J = |
| | | 12.6 Hz, 2H), 1.51 (q, J = 10.3, |
| | | 9.9 Hz, 2H), 0.39 (dt, J = 6.2, |
| | | 3.0 Hz, 2H), 0.28-0.20 (m, 2H). |
| 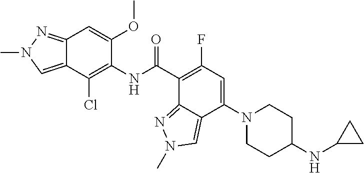 [SMILES: COc1cc2nn(C)cc2c(Cl)c1NC(=O)c1c(F)cc(N2CCC(NC3CC3)CC2)c2cn(C)nc12] Compound 445 | 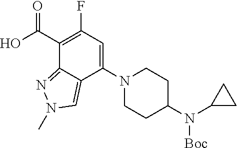 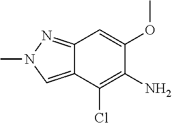 C209 | Modification: Step 1 was ran for 2 h at room temperature. Step 2 was ran for 2 h at room temperature. The crude product was purified by Prep-HPLC (Condition 2, Gradient 12) to afford N-(4- chloro-6-methoxy-2-methyl- indazol-5-yl)-4-[4- (cyclopropylamino)-1- piperidyl]-6-fluoro-2-methyl- indazole-7-carboxamide 2,2,2- trifluoroacetate (Compound 445, 33.9 mg, 64.5 μmol, 58% yield) as a solid. LCMS (ES, m/z): 526 [M + H]⁺. ¹H NMR (400 MHz, DMSO-d₆) δ 10.40 |
| | | (s, 1H), 8.84 (s, 1H), 8.82 (s, |
| | | 2H), 8.37 (s, 1H), 7.05 (s, 1H), |
| | | 6.32 (d, J = 15.4 Hz, 1H), 4.20 |
| | | (s, 3H), 4.16 (s, 3H), 4.07 (d, |
| | | J = 13.2 Hz, 2H), 3.81 (s, 3H), |
| | | 3.56-3.53 (m, 1H), 3.09 (t, J = |
| | | 12.4 Hz, 2H), 2.83-2.76 (m, |
| | | 1H), 2.19 (d, J = 11.7 Hz, 2H), |
| | | 1.80-1.67 (m, 2H), 0.87-0.84 |
| | | (m, 4H). |

### Example 26: Synthesis of A110

### Synthesis of Intermediate A105

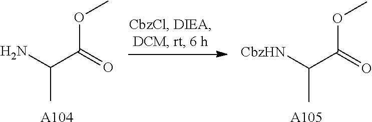

To a stirred solution of methyl alaninate hydrochloride (A104, 20 g, 143.3 mmol) in DCM (200 mL) was added DIEA (55.56 g, 429.9 mmol) at room temperature. Benzyl carbonochloridate (26.9 g, 157.6 mmol) was then added dropwise over 40 min, the mixture was stirred for 6 h at rt, and diluted with DCM (200 mL). The organic layer was washed with 10% citric acid (400 mL), sat. NaHCO₃, brine (200 mL), dried over anhydrous Na₂SO₄, and filtered. The filtrate was concentrated under reduced pressure and triturated with n-hexane to afford methyl 2-(benzyloxycarbonylamino) propanoate (A105, 35 g, 118.0 mmol). LCMS (ES, m/z): 238 [M+H]⁺.

### Synthesis of Intermediate A106

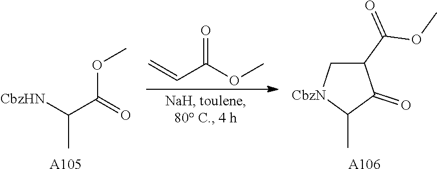

To a stirred mixture of Intermediate A105 (30 g, 126.5 mmol) and methyl prop-2-enoate (13.06 g, 151.7 mmol, 13.7 mL) in toluene (600 mL) was added NaH (60% dispersion in oil) (3.03 g, 126.5 mmol) in portions at 0° C. The resulting mixture was stirred for 4 h at 80° C., allowed to cool to room temperature, quenched by the addition of 10% citric acid (300 mL), and extracted with MTBE (3×300 mL). The combined organic layers were washed with water (300 mL), dried over anhydrous Na₂SO₄, filtered, and concentrated under reduced pressure to afford 01-benzyl O3-methyl 5-methyl-4-oxo-pyrrolidine-1,3-dicarboxylate (A106, 46 g, 110.5 mmol). LCMS (ES, m/z): 292 [M+H]⁺.

### Synthesis of Intermediate A107

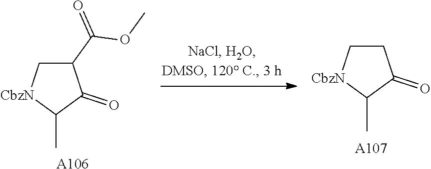

To a stirred solution of Intermediate A106 (40 g, 137.3 mmol) in DMSO (300 mL) were added H₂O (100 mL) and NaCl (32.1 g, 549.3 mmol, 14.8 mL), and the reaction was stirred for 3 h at 120° C. The resulting mixture was allowed to cool to room temperature, diluted with water (200 ml), and extracted with MTBE (3×400 mL). The combined organic layers were dried over anhydrous Na₂SO₄, and filtered. The filtrate was concentrated under reduced pressure and purified by silica gel column chromatography (25% THF in PE) to afford benzyl 2-methyl-3-oxo-pyrrolidine-1-carboxylate (A107, 20 g, 85.7 mmol). LCMS (ES, m/z): 234 [M+H]⁺.

### Synthesis of Intermediate A108

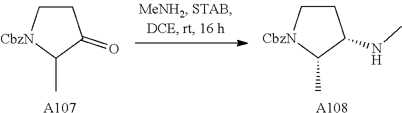

To a stirred solution of Intermediate A107 (7 g, 30.01 mmol) in DCE (140 mL) was added methanamine (1.86 g, 60.02 mmol), and the reaction was stirred for 1 h at room temperature. NaBH(OAc)₃ (19.08 g, 90.03 mmol) was then added to the reaction mixture, and the reaction was stirred for 16 h. The resulting mixture was quenched by the addition of sat. NH₄Cl (100 mL) and extracted with DCM (3×300 mL). The combined organic layers were washed with brine (300 mL), dried over anhydrous Na₂SO₄, and filtered. The filtrate was concentrated under reduced pressure and purified by silica gel column chromatography (9% MeOH in DCM) to afford benzyl 2-methyl-3-(methylamino)pyrrolidine-1-carboxylate (A108, 4 g, 16.11 mmol). LCMS (ES, m/z): 249 [M+H]⁺.

### Synthesis of Intermediate A109

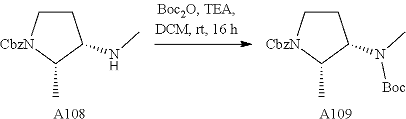

To a stirred solution of Intermediate A108 (460 mg, 1.85 mmol) in DCM (4.6 mL) were added di-tert-butyl dicarbonate (808.6 mg, 3.70 mmol) and TEA (281.2 mg, 2.78 mmol), and the reaction was stirred for 2 h at room temperature. The resulting mixture was concentrated under reduced pressure and purified by silica gel column chromatography (14% THF in PE) to afford benzyl 3-[tert-butoxycarbonyl(methyl)amino]-2-methyl-pyrrolidine-1-carboxylate (A109, 340 mg, 975.8 μmol). LCMS (ES, m/z): 349 [M+H]⁺.

### Synthesis of Intermediate A110

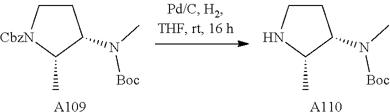

To a stirred solution of Intermediate A109 (300 mg, 861 μmol) in THF (6 mL) was added Pd/C (18 mg, 172.2 μmol) at room temperature. The resulting mixture was stirred for 2 h at under hydrogen atmosphere, filtered, and concentrated under reduced pressure to afford tert-butyl N-methyl-N-(2-methylpyrrolidin-3-yl)carbamate (A110, 210 mg, 685.9 μmol). LCMS (ES, m/z): 215 [M+H]⁺.

### Example 27: Synthesis of Compound 194

### Synthesis of Intermediate A113

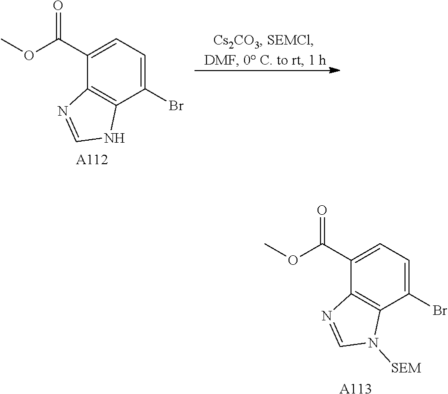

To a stirred mixture of methyl 7-bromo-1H-benzimidazole-4-carboxylate (2.4 g, 9.41 mmol) and Cs₂CO₃ (6.1 g, 18.82 mmol) in DMF (50 mL) was added SEMCl (2.35 g, 14.17 mmol) dropwise at room temperature. The resulting mixture was stirred for 2 h while being monitored by LCMS, then diluted with water (200 mL) and extracted with EtOAc (2×200 mL). The combined organic layers were washed with water (2×100 mL), dried over anhydrous Na₂SO₄, filtered, and concentrated under reduced pressure to afford methyl 7-bromo-1-(2-trimethylsilylethoxymethyl)benzimidazole-4-carboxylate (A113, 3.0 g, 7.8 mmol). LCMS (ES, m/z): 385 [M+H]⁺.

### Synthesis of Intermediate A114

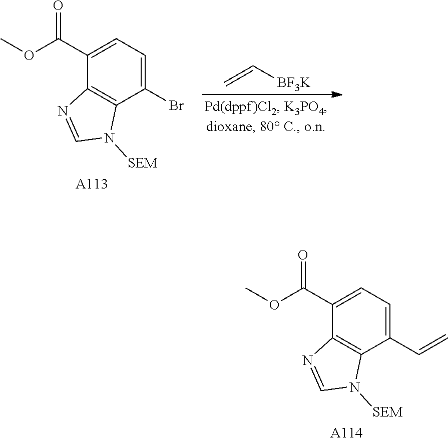

To a solution of Intermediate A113 (1.7 g, 4.41 mmol) and potassium vinyltrifluoroborate (886.4 mg, 6.62 mmol) in dioxane (30 mL) and H₂O (5 mL) were added K₃PO₄ (1.8 g, 8.82 mmol) and Pd(dppf)Cl₂ (322.8 mg, 441.2 μmol), and the reaction was stirred for 2 h at 80° C. under N₂. The resulting mixture was concentrated under reduced pressure and purified by silica gel column chromatography (33% EtOAc in PE) to afford methyl 1-(2-trimethylsilylethoxymethyl)-7-vinyl-benzimidazole-4-carboxylate (A114, 1.3 g, 3.9 mmol). LCMS (ES, m/z): 333 [M+H]⁺.

### Synthesis of Intermediate A115

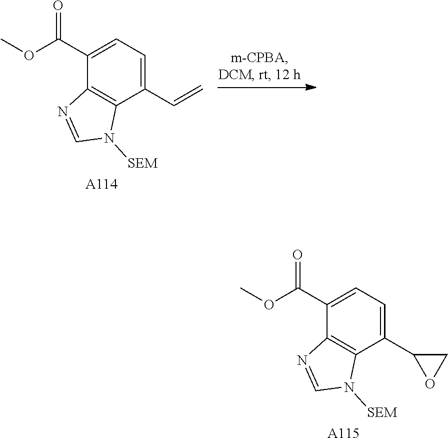

To a stirred mixture of Intermediate A114 (1.2 g, 3.61 mmol) in DCM (40 mL) was added m-CPBA (0.93 g, 5.41 mmol) in portions. The resulting mixture was stirred for 12 h at room temperature, diluted with H₂O (100 mL), and then extracted with DCM (100 mL). The combined organic layers were washed with brine (100 mL), dried over anhydrous Na₂SO₄, and filtered. The filtrate was concentrated under reduced pressure and purified by silica gel column chromatography (25% EtOAc in PE) to afford methyl 7-(oxiran-2-yl)-1-(2-trimethylsilylethoxymethyl)benzimidazole-4-carboxylate (A115, 600 mg, 1.7 mmol). LCMS (ES, m/z): 349 [M+H]⁺.

### Synthesis of Intermediate A116

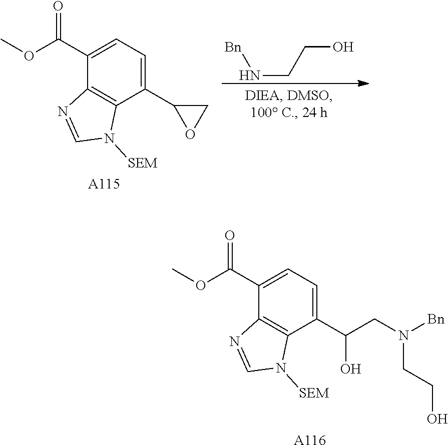

To a stirred solution of Intermediate A115 (600.0 mg, 1.72 mmol) and DIEA (1112.6 mg, 8.61 mmol) in DMSO (10 mL) was added 2-(benzylamino) ethanol (650.8 mg, 4.31 mmol) at room temperature. The resulting mixture was stirred for 24 h at 100° C. while being monitored by LCMS, then diluted with water (30 mL), and extracted with EtOAc (2×30 mL). The combined organic layers were washed with water (2×30 mL), dried over anhydrous Na₂SO₄, and filtered. The filtrate was concentrated under reduced pressure and purified by silica gel column chromatography (50% EtOAc in PE) to afford methyl 7-[2-[benzyl(2-hydroxyethyl)amino]-1-hydroxy-ethyl]-1-(2-trimethylsilylethoxymethyl)benzimidazole-4-carboxylate (A116, 500 mg, 1 mmol). LCMS (ES, m/z): 500 [M+H]⁺.

### Synthesis of Intermediate A117

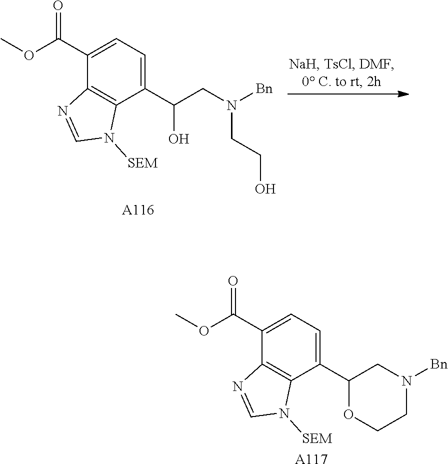

A solution of Intermediate A116 (500.0 mg, 1 mmol) in DMF (10 mL) was treated with NaH (120.0 mg, 3 mmol) for 2 h at 0° C. under N₂ followed by the addition of TsCl (105.8 mg, 1.5 mmol) in portions. The resulting mixture was stirred for 1 h at while being monitored by LCMS. The reaction was quenched by the addition of sat. NH₄Cl (aq.) (30 mL) at 0° C. and then extracted with EtOAc (2×30 mL). The combined organic layers were washed with water (2×30 mL), dried over anhydrous Na₂SO₄, filtered, and concentrated under reduced pressure to afford methyl 7-(4-benzylmorpholin-2-yl)-1-(2-trimethylsilylethoxymethyl)benzimidazole-4-carboxylate (A117, 120.0 mg, 249.1 μmol). LCMS (ES, m/z): 482 [M+H]⁺.

### Synthesis of Intermediate A118

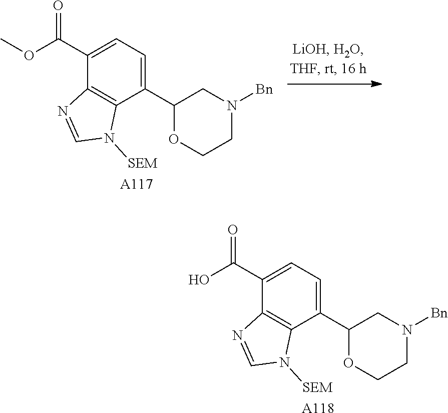

To a stirred mixture of Intermediate A117 (120.0 mg, 249.1 μmol) in THF (2 mL) and H₂O (2 mL) was added LiOH (29.8 mg, 1.25 mmol). The resulting mixture was stirred for 16 h at 25° C. while being monitored by LCMS, then diluted with water (5 mL), acidified to pH 6 with HCl (aq.), and extracted with EtOAc (2×5 mL). The combined organic layers were washed with brine (2×5 mL), dried over anhydrous Na₂SO₄, filtered, and concentrated under reduced pressure to afford 7-(4-benzylmorpholin-2-yl)-1-(2-trimethylsilylethoxymethyl)-benzimidazole-4-carboxylic acid (A118, 100 mg, 213.8 μmol). LCMS (ES, m/z): 468 [M+H]⁺.

### Synthesis of Intermediate A119

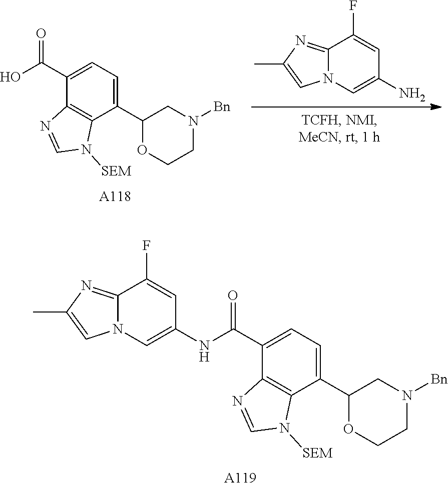

To a stirred mixture of Intermediate A118 (90.0 mg, 192.5 μmol) and 8-fluoro-2-methyl-imidazo[1,2-a]pyridin-6-amine (31.8 mg, 192.5 μmol) in MeCN (2 mL) was added TCFH (216.0 mg, 769.8 μmol), and the reaction was stirred for 1 h at room temperature. The resulting mixture was diluted with water (10 mL) and extracted with EtOAc (2×10 mL). The combined organic layers were washed with brine (2×10 mL), dried over anhydrous Na₂SO₄, filtered, and concentrated under reduced pressure to afford 7-(4-benzylmorpholin-2-yl)-N-(8-fluoro-2-methyl-imidazo[1,2-a]pyridin-6-yl)-1-(2-trimethylsilylethoxymethyl)benzimidazole-4-carboxamide (A119, 90.0 mg, 146.4 μmol). LCMS (ES, m/z): 615 [M+H]⁺.

### Synthesis of Intermediate A120

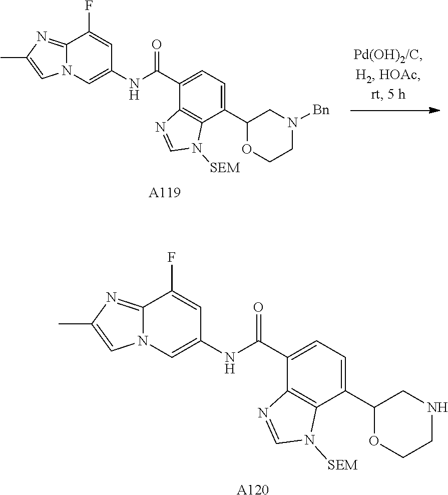 [SMILES: Cc1cn2cc(NC(=O)c3ccc(C4CN(Cc5ccccc5)CCO4)c4c3ncn4COCC[Si](C)(C)C)cc(F)c2n1 ; "A119" ; Cc1cn2cc(NC(=O)c3ccc(C4CNCCO4)c4c3ncn4COCC[Si](C)(C)C)cc(F)c2n1 ; "A120"]

To a solution of Intermediate A119 (50.0 mg, 81.33 μmol) in HOAc (1 mL) was added Pd(OH)₂ 20% on Carbon (wetted with ca. 55% water) (22.8 mg, 162.6 μmol) in a pressure tank. The mixture was hydrogenated at room temperature under 30 psi of hydrogen pressure for 5 h, filtered through a Celite pad, and then concentrated under reduced pressure to afford N-(8-fluoro-2-methyl-imidazo[1,2-a]pyridin-6-yl)-7-morpholin-2-yl-1-(2-trimethylsilylethoxymethyl)-benzimidazole-4-carboxamide (A120, 35 mg, 66.7 μmol). LCMS (ES, m/z): 525 [M+H]⁺.

### Synthesis of Compound 194

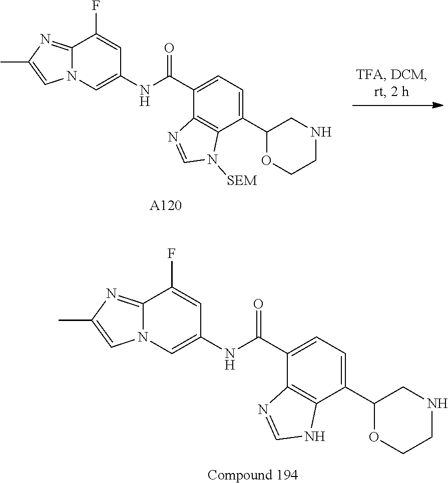 [SMILES: Cc1cn2cc(NC(=O)c3ccc(C4CNCCO4)c4c3ncn4COCC[Si](C)(C)C)cc(F)c2n1 ; "A120" ; Cc1cn2cc(NC(=O)c3ccc(C4CNCCO4)c4[nH]cnc34)cc(F)c2n1 ; "Compound 194"]

To a stirred mixture of Intermediate A120 (30.0 mg, 57.18 μmol) in DCM (1 mL) was added TFA (0.5 mL), and the reaction was stirred for 2 h at room temperature. The resulting mixture was concentrated under reduced pressure and purified by Prep-HPLC (Condition 2, Gradient 2) to afford N-(8-fluoro-2-methyl-imidazo[1,2-a]pyridin-6-yl)-7-morpholin-2-yl-1H-benzimidazole-4-carboxamide (Compound 194, 5.4 mg, 13.69 μmol). LCMS (ES, m/z): 395 [M+H]⁺. ¹H NMR (400 MHz, DMSO-d₆) δ 9.34 (s, 1H), 8.56 (s, 1H), 8.14 (d, J=2.4 Hz, 1H), 8.02 (d, J=7.8 Hz, 1H), 7.76 (d, J=12.0 Hz, 1H), 7.45 (d, J=7.9 Hz, 1H), 5.32 (d, J=11.0 Hz, 1H), 4.27-4.18 (m, 1H), 4.07-3.96 (m, 1H), 3.71-3.63 (m, 1H), 3.38-3.18 (m, 3H), 2.44 (s, 3H).

### Example 28: Synthesis of Compound 196

### Synthesis of Intermediate A128

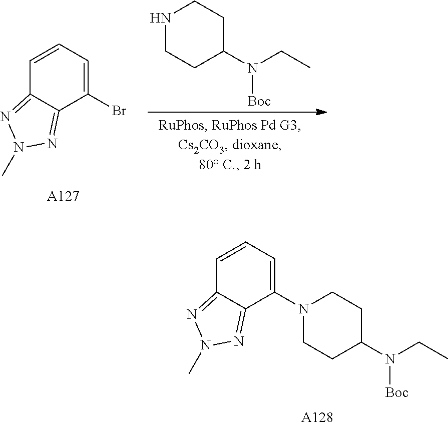 [SMILES: Cn1nc2cccc(Br)c2n1 ; CCN(C(=O)OC(C)(C)C)C1CCNCC1 ; "A127" ; CCN(C(=O)OC(C)(C)C)C1CCN(c2cccc3nn(C)nc23)CC1 ; "A128"]

To a stirred mixture of 4-bromo-2-methyl-1,2,3-benzotriazole (A127, 350 mg, 1.65 mmol, 1 eq) and tert-butyl N-ethyl-N-(piperidin-4-yl)carbamate (452 mg, 1.98 mmol, 1.2 eq) in 1,4-dioxane (7 mL) were added RuPhos (154 mg, 0.33 mmol, 0.2 eq), Cs₂CO₃ (1.08 g, 3.30 mmol, 2 eq) and RuPhos Pd G3 (138 mg, 165.1 μmol, 0.1 eq) in portions, and the reaction was stirred for 2 h at 80° C. under N₂. The resulting mixture was allowed to cool to room temperature, concentrated under reduced pressure and purified by silica gel column chromatography (33% EtOAc in PE) to afford tert-butyl N-ethyl-N-[1-(2-methyl-1,2,3-benzotriazol-4-yl)piperidin-4-yl]carbamate (A128, 280 mg). LCMS (ES, m/z): 360 [M+H]⁺.

### Synthesis of Intermediate A129

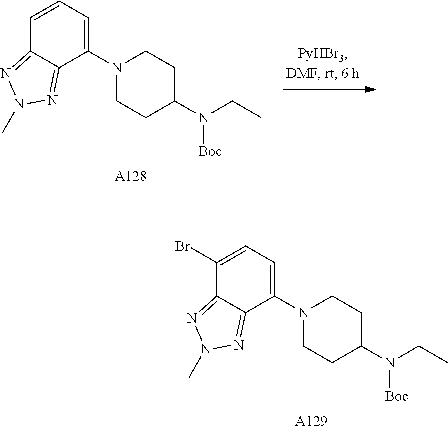 [SMILES: CCN(C(=O)OC(C)(C)C)C1CCN(c2cccc3nn(C)nc23)CC1 ; "A128" ; CCN(C(=O)OC(C)(C)C)C1CCN(c2ccc(Br)c3nn(C)nc23)CC1 ; "A129"]

To a stirred solution of Intermediate A128 (270 mg, 0.75 mmol, 1 eq) in DMF (5 mL) was added PyHBr₃ (250 mg, 0.75 mmol, 1 eq) at 0° C. under N₂. The resulting mixture was stirred for 6 h at room temperature, diluted with water (10 mL), and then extracted with EtOAc (2×30 mL). The combined organic layers were washed with brine (30 mL), dried over anhydrous Na₂SO₄, and filtered. The filtrate was concentrated under reduced pressure and purified by silica gel column chromatography (20% EtOAc in PE) to afford tert-butyl N-[1-(7-bromo-2-methyl-1,2,3-benzotriazol-4-yl)piperidin-4-yl]-N-ethylcarbamate (A129, 280 mg). LCMS (ES, m/z): 438 [M+H]⁺.

### Synthesis of Intermediate A130

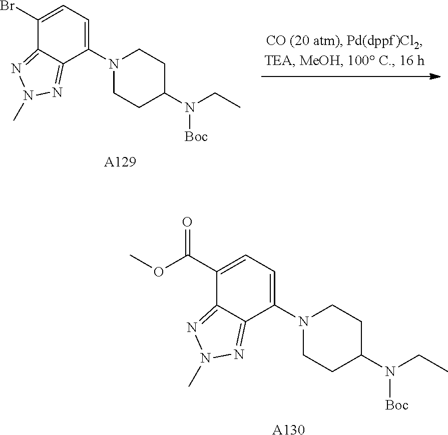 [SMILES: CCN(C(=O)OC(C)(C)C)C1CCN(c2ccc(Br)c3nn(C)nc23)CC1 ; "A129" ; CCN(C(=O)OC(C)(C)C)C1CCN(c2ccc(C(=O)OC)c3nn(C)nc23)CC1 ; "A130"]

To a solution of Intermediate A129 (280 mg, 0.64 mmol, 1 eq) and TEA (194 mg, 1.92 mmol, 3 eq) in MeOH (5 mL) was added Pd(dppf)Cl₂ (47 mg, 0.06 mmol, 0.1 eq) in a pressure tank. The mixture was purged with nitrogen, pressurized to 20 atm with carbon monoxide at 100° C. for 16 h, cooled to room temperature and filtered to remove insoluble solids. The filtrate was concentrated under reduced pressure and purified by silica gel column chromatography (50% EtOAc in PE) to afford methyl 7-(4-[(tert-butoxycarbonyl)(ethyl)amino]piperidin-1-yl)-2-methyl-1,2,3-benzotriazole-4-carboxylate (A130, 200 mg). LCMS (ES, m/z): 418 [M+H]⁺.

### Synthesis of Intermediate A131

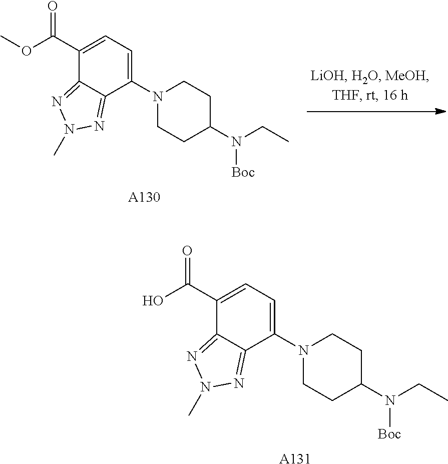 [SMILES: CCN(C(=O)OC(C)(C)C)C1CCN(c2ccc(C(=O)OC)c3nn(C)nc23)CC1 ; "A130" ; CCN(C(=O)OC(C)(C)C)C1CCN(c2ccc(C(=O)O)c3nn(C)nc23)CC1 ; "A131"]

To a stirred mixture of Intermediate A130 (180 mg, 0.43 mmol, 1 eq) in water (2 mL), THF (2 mL) and MeOH (0.4 mL) was added LiOH (52 mg, 2.16 mmol, 5 eq), and the reaction was stirred for 16 h at room temperature. The resulting mixture was acidified to pH 5 with HCl (1 M) and extracted with DCM (3×10 mL). The combined organic layers were washed with brine (10 mL), dried over anhydrous Na₂SO₄, filtered, and concentrated under reduced pressure to afford 7-(4-[(tert-butoxycarbonyl)(ethyl)amino]piperidin-1-yl)-2-methyl-1,2,3-benzotriazole-4-carboxylic acid (A131, 160 mg). LCMS (ES, m/z): 404 [M+H]⁺.

### Synthesis of Intermediate A132

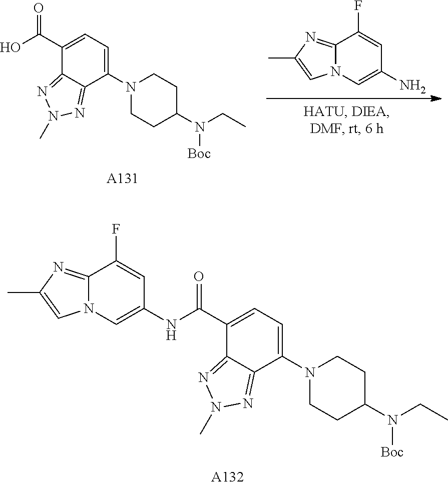 [SMILES: CCN(C(=O)OC(C)(C)C)C1CCN(c2ccc(C(=O)O)c3nn(C)nc23)CC1 ; Cc1cn2cc(N)cc(F)c2n1 ; "A131" ; CCN(C(=O)OC(C)(C)C)C1CCN(c2ccc(C(=O)Nc3cc(F)c4nc(C)cn4c3)c3nn(C)nc23)CC1 ; "A132"]

A mixture of Intermediate A131 (120 mg, 0.3 mmol, 1 eq), HATU (170 mg, 0.45 mmol, 1.5 eq) and DIEA (115 mg, 0.89 mmol, 3 eq) in DMF (4 mL) was stirred for 5 min at room temperature. 8-fluoro-2-methylimidazo[1,2-a]pyridin-6-amine dihydrochloride (59 mg, 0.36 mmol, 1.2 eq) was then added. The resulting mixture was stirred for an additional 6 h at room temperature and quenched with water (10 mL). The precipitated solids were collected by filtration and washed with water (2×5 mL) to afford tert-butyl N-ethyl-N-(1-[7-((8-fluoro-2-methylimidazo[1,2-a]pyridin-6-yl)carbamoyl)-2-methyl-1,2,3-benzotriazol-4-yl]piperidin-4-yl)-carbamate (A132, 95 mg). LCMS (ES, m/z): 550 [M+H]⁺.

### Synthesis of Compound 196

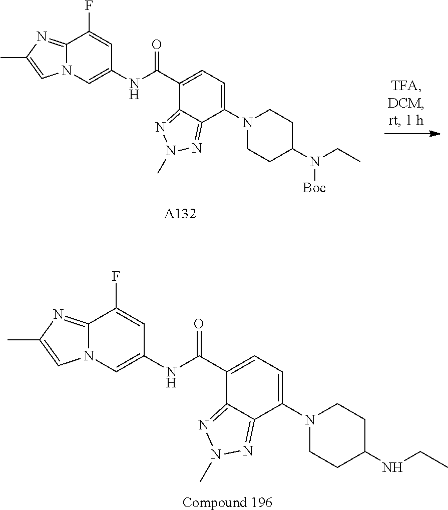

To a stirred Intermediate A132 (85 mg, 0.15 mmol, 1 eq) in DCM (2 mL) was added TFA (1 mL), and the reaction was stirred for 1 h at room temperature. The resulting mixture was concentrated under reduced pressure and purified by reverse flash chromatography (Condition 5, Gradient 1) to afford 7-[4-(ethylamino)piperidin-1-yl]-N-(8-fluoro-2-methyl-imidazo[1,2-a]pyridin-6-yl)-2-methyl-1,2,3-benzotriazole-4-carboxamide (Compound 196, 10.1 mg). LCMS (ES, m/z): 451 [M+H]⁺. ¹H NMR (400 MHz, DMSO-d₆) δ 10.17 (s, 1H), 9.19 (d, J=1.6 Hz, 1H), 8.00 (d, J=8.3 Hz, 1H), 7.91 (d, J=3.0 Hz, 1H), 7.39 (d, J=12.5 Hz, 1H), 6.73 (d, J=8.4 Hz, 1H), 4.62 (s, 3H), 4.46 (d, J=13.1 Hz, 2H), 3.23 (d, J=12.0 Hz, 2H), 2.72 (s, 1H), 2.61 (q, J=7.0 Hz, 2H), 2.36 (s, 3H), 1.96 (d, J=12.8 Hz, 2H), 1.38 (d, J=11.9 Hz, 2H), 1.03 (t, J=7.1 Hz, 3H).

An analogous method was followed to obtain the following compounds.

| | Starting | |
|---|---|---|
| Compound | Material | Characterization |
| 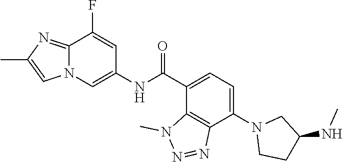 Compound 160 | 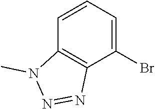 A136 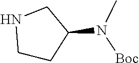 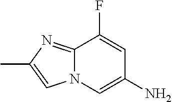 | Modifications: Step 2 was ran at −10° C. for 1 h. Step 3 was ran at 110° C. Step 5 was ran using TCFH (1.5 eq) and NMI (3 eq) in MeCN for 16 h. The resulting mixture was concentrated under reduced pressure and purified by reversed- phase flash chromatography (Condition 3, Gradient 9) to afford N-(8-fluoro-2-methyl- imidazo[1,2-a]pyridin-6-yl)-3-methyl-7-[(3S)- 3-(methylamino)-pyrrolidin-1- yl]benzotriazole-4-carboxamide (Compound 160, 42 mg, 99.42 μmol). LCMS (ES, m/z): 423 [M + H]⁺. ¹H NMR (300 MHz, DMSO-d₆) δ 10.38 (s, 1H), 9.12 (d, J = 1.6 Hz, 1H), 7.96- 7.89 (m, 1H), 7.73 (d, J = 8.4 Hz, 1H), 7.29 (dd, J = 12.8, 1.6 Hz, 1H), 6.27 (d, J = 8.4 Hz, 1H), 4.30 (s, 3H), 3.85 (dd, J = 45.9, 36.7 Hz, 4H), 3.36 (d, J = 5.2 Hz, 1H), 2.54 (d, J = 5.6 Hz, 1H), 2.35 (s, 6H), 2.16 (dd, J = 12.5, 6.0 Hz, |
| | | 1H), 1.97-1.87 (m, 1H). |
| 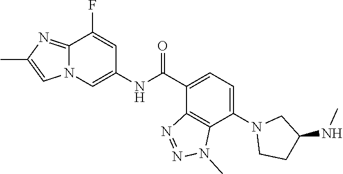 Compound 166 | 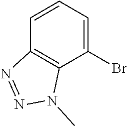 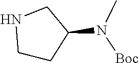 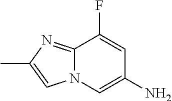 | Modifications: Step 2 was ran at −10° C. for 1 h. Step 3 was ran at 110° C. Step 5 was ran using TCFH (1.5 eq) and NMI (3 eq) in MeCN for 16 h. The residue was purified by reversed-phase flash chromatography (Condition 3, Gradient 9) to afford (S)-N-(8-fluoro-2-methylimidazo- [1,2-a]pyridin-6-yl)-1-methyl-7-(3- (methylamino)pyrrolidin-1-yl)-1H-benzo[d]- [1,2,3 ]triazole-4-carboxamide (Compound 166, 37.2 mg). LCMS (ES, m/z): 423 [M + H]⁺. ¹H NMR (300 MHz, DMSO-d₆) δ 10.90 (s, 1H), 9.16 (d, J = 1.6 Hz, 1H), 8.00-7.88 (m, 2H), 7.38 (dd, J = 12.4, 1.7 Hz, 1H), 7.04 (d, J = 8.2 Hz, 1H), 4.57 (s, 3H), 3.58-3.35 (m, 4H), 3.21 (dd, J = 9.6, 4.3 Hz, 1H), 2.33 (d, J = 13.7 Hz, 6H), 2.16 (dq, J = 13.5, 6.9 Hz, 1H), 1.94 (s, 1H), 1.79 (dq, J = 12.4, 6.7 Hz, 1H). |
| 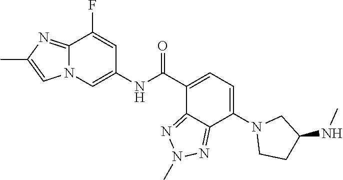 Compound 163 | 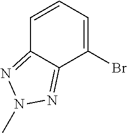 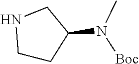 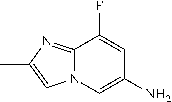 | Modifications: Step 2 was ran at −10° C. for 2 h. Step 3 was ran at 110° C. Step 5 was ran using TCFH (1.5 eq) and NMI (3 eq) in MeCN for 2 h. The residue was purified by reversed-phase flash chromatography (Condition 3, Gradient 9) to afford (S)-N-(8-fluoro-2-methylimidazo- [1,2-a]pyridin-6-yl)-2-methyl-7-(3- (methylamino)pyrrolidin-1-yl)-2H-benzo[d]- [1,2,3 ]triazole-4-carboxamide (Compound 163, 7.7 mg, 18.3 μmol). LCMS (ES, m/z): 423 [M + H]⁺. ¹H NMR (300 MHz, DMSO-d₆) δ 10.07 (s, 1H), 9.17 (d, J = 1.6 Hz, 1H), 7.99 (d, J = 8.3 Hz, 1H), 7.89 (d, J = 3.1 Hz, 1H), 7.36 (dd, J = 12.5, 1.6 Hz, 1H), 6.29 (d, J = 8.4 Hz, 1H), 4.59 (s, 3H), 3.90 (s, 1H), 3.81 (s, 2H), 3.62 (d, J = 11.6 Hz, 1H), 3.35 (s, 1H), 2.34 (d, J = 6.3 Hz, 6H), 2.13 (dt, J = 13.2, 6.3 Hz, 1H), 1.96 (d, J = 7.8 Hz, 1H), 1.90 (dd, J = 12.1, 6.4 Hz, 1H). |
| 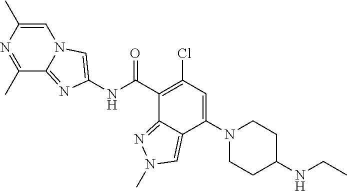 [SMILES: CCNC1CCN(c2cc(Cl)c(C(=O)Nc3cn4cc(C)nc(C)c4n3)c3nn(C)cc23)CC1] Compound 348 | 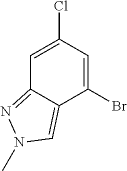 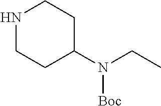 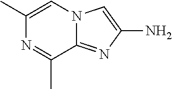 | Modifications: Step 1 was ran at 90° C. for 2 h. Step 2 was ran at −10° C. for 2 h. Step 3 was ran at 110° C. Step 4 was ran at 50° C. for 1 h. Step 5 was ran using TCFH (1.5 eq) and NMI (3 eq) in MeCN at 50° C. for 2 h. The residue was purified by reversed-phase flash chromatography (Condition 3, Gradient 6) to afford 6-chloro-N-(6,8-dimethylimidazo- [1,2-a]pyrazin-2-yl)-4-[4-(ethylamino)-1- piperidyl]-2-methyl-indazole-7-carboxamide (Compound 348, 27.1 mg, 56.3 μmol, 37% yield) as a solid. LCMS (ES, m/z): 481 [M + H]⁺. ¹H NMR (400 MHz, DMSO-d₆) δ 11.48 (s, 1H), 8.67 (s, 1H), 8.32 (d, J = 12.9 Hz, 2H), 6.32 (s, 1H), 4.17 (s, 3H), 3.80 (d, J = 12.9 Hz, 2H), 3.00 (t, J = 11.5 Hz, 2H), 2.67 (s, 3H), 2.60 (q, J = 7.0 Hz, 3H), 2.38 (s, 3H), 1.95 (d, J = 12.4 Hz, 2H), 1.55 (s, 1H), 1.43 (q, J = 10.9 Hz, 2H), 1.04 (t, J = 7.1 Hz, 3H). |
| 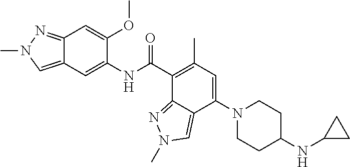 [SMILES: COc1cc2nn(C)cc2cc1NC(=O)c1c(C)cc(N2CCC(NC3CC3)CC2)c2cn(C)nc12] Compound 472 | 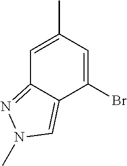 D9 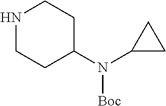 [SMILES: CC(C)(C)OC(=O)N(C1CCNCC1)C1CC1] 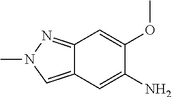 | Modifications: Step 1 was ran for 16 h. Step 2 was ran at 0° C. for 2 h. Step 3 was ran at 120° C. Step 4 was ran at 60° C. Step 5 was ran using TCFH (1.5 eq) and NMI (3 eq) in MeCN for 2 h. Step 6 was ran for 2 h. The reaction mixture was concentrated under reduced pressure and purified by Prep-HPLC (Condition 6, Gradient 10) to afford 4-[4- (cyclopropylamino)-1-piperidyl]-N-(6- methoxy-2-methyl-indazol-5-yl)-2,6- dimethylindazole-7-carboxamide (Compound 472, 30.1 mg, 61.7 μmol). LCMS (ES, m/z): 488 [M + H]⁺ ¹H NMR (400 MHz, DMSO-d₆) δ 11.88 (s, 1H), 8.84 (s, 1H), 8.64 (s, 1H), 8.15 (s, 1H), 7.04 (s, 1H), 6.27 (s, 1H), 4.25 (s, 3H), 4.09 (s, 3H), 4.02 (s, 3H), 3.88-3.80 (m, 2H), 3.04-2.93 (m, 2H), 2.84-2.80 (m, 1H), 2.76 (s, 3H), 2.16 (dd, J = 7.1, 3.7 Hz, 1H), 2.05-1.96 (m, 2H), 1.52-1.46 (m, 2H), 0.42 (dt, J = 6.3, 3.1 Hz, 2H), 0.26 (p, J = 3.9 Hz, 2H). |
| 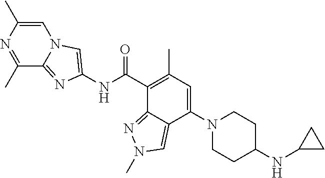 [SMILES: Cc1cn2cc(NC(=O)c3c(C)cc(N4CCC(NC5CC5)CC4)c4cn(C)nc34)nc2c(C)n1] Compound 473 | 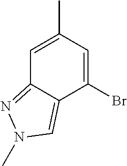 D9 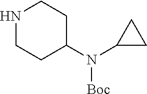 [SMILES: CC(C)(C)OC(=O)N(C1CCNCC1)C1CC1] 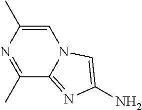 | Modifications: Step 2 was ran at −10° C. for 1 h. Step 3 was ran at 110° C. Step 5 was ran using TCFH (1.5 eq) and NMI (3 eq) in MeCN for 2 h. Step 6 was ran for 2 h. The crude product was purified by Prep-HPLC (Condition 6, Gradient 10) to afford 4-[4- (cyclopropylamino)-1-piperidyl]-N-(6,8- dimethylimidazo[1,2-a]pyrazin-2-yl)-2,6- dimethyl-indazole-7-carboxamide (Compound 473, 10.2 mg, 21.6 μmol). LCMS (ES, m/z): 473 [M + H]'⁺. ¹H NMR (400 MHz, DMSO-d₆) δ 11.97 (s, 1H), 8.68 (s, 1H), 8.34 (s, 1H), 8.28 (s, 1H), 6.30 (s, 1H), 4.25 (s, 3H), 3.86 (d, J = 12.7 Hz, 2H), 3.02 (t, J = 11.6 Hz, 2H), 2.76-2.75 (m, 1H), 2.75 (s, 3H), 2.69 (s, 3H), 2.36 (s, 3H), 2.25 (s, 1H), 2.13 (tt, J = 6.7, 3.5 Hz, 1H), 2.00 (d, J = 12.1 Hz, 2H), 1.49- 1.46 (m, 1H), 0.40 (dt, J = 6.2, 3.1 Hz, 2H), 0.28-0.20 (m, 2H). |
| 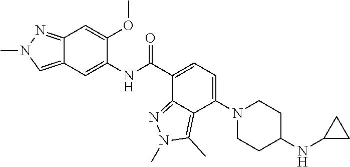 [SMILES: COc1cc2nn(C)cc2cc1NC(=O)c1ccc(N2CCC(NC3CC3)CC2)c2c(C)n(C)nc12] Compound 485 | 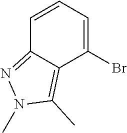 D47 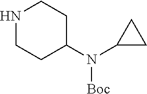 [SMILES: CC(C)(C)OC(=O)N(C1CCNCC1)C1CC1] 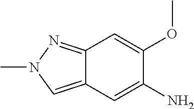 | Modifications: Step 2 was ran in DCM at 0° C. for 1.5 h. Step 3 was ran at 120° C. Step 4 was ran for 3 h. Step 5 was ran using TCFH (1.5 eq) and NMI (3 eq) in MeCN for 2 h. Step 6 was ran using HCl in dioxane and DCM for 2 h at room temperature. The crude product was purified by Prep-HPLC (Condition 5, Gradient 8) to afford 4-[4- (cyclopropylamino)-1-piperidyl]-N-(6- methoxy-2-methyl-indazol-5-yl)-2,3- dimethyl-indazole-7-carboxamide (Compound 485, 15 mg, 30.8 μmol). LCMS (ES, m/z): 488 [M + H]⁺. ¹H NMR (400 MHz, DMSO-d₆) δ 11.65 (s, 1H), 8.82 (s, 1H), 8.17 (s, 1H), 7.96 (d, J = 7.8 Hz, 1H), 7.05 (s, 1H), 6.59 (d, J = 7.9 Hz, 1H), 4.14 (s, 3H), 4.06 (s, 3H), 4.03 (s, 3H), 3.39-3.34 (m, 2H), 2.77 (s, 3H), 2.75-2.69 (m, 2H), 2.24 (s, 1H), 2.11 (s, 1H), 2.01 (d, J = 12.3 Hz, 2H), 1.52 (q, J = 11.3, 10.8 Hz, 2H), 0.42-0.34 (m, 2H), 0.26-0.18 (m, 2H). |
| 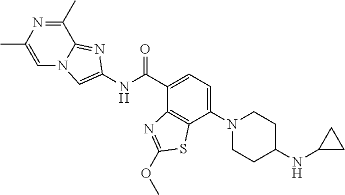 [SMILES: COc1nc2c(C(=O)Nc3cn4cc(C)nc(C)c4n3)ccc(N3CCC(NC4CC4)CC3)c2s1] Compound 667 | 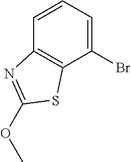 A1 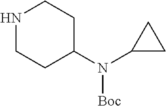 [SMILES: CC(C)(C)OC(=O)N(C1CCNCC1)C1CC1] Boc 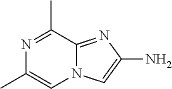 | Modifications: Step 1 was ran using Pd- PEPPSI-IPentCl, at 90° C. for 5 h. Step 2 was ran at room temperature for 2 h. Step 3 was ran at 80° C. Step 4 was ran for 2 h. Step 5 was ran using TCFH (1.5 eq) and NMI (3 eq) in MeCN for 2 h. Step 6 was ran for 2 h. The residue was purified by reversed-phase flash chromatography (Condition 6, Gradient 3) to afford 7-(4-(cyclopropylamino)- piperidin-1-yl)-N-(6,8-dimethylimidazo[1,2- a]pyrazin-2-yl)-2-methoxy-benzo[d]thiazole- 4-carboxamide 2,2,2-trifluoroacetate (Compound 667, 0.01 g, 13.2 μmol). LCMS (ES, m/z): 492 [M + H]⁺. ¹H NMR (300 MHz, Methanol-d₄) δ 8.59 (s, 1H), 8.41 (s, 1H), 8.30 (d, J = 8.6 Hz, 1H), 7.16 (d, J = 8.6 Hz, 1H), 4.50 (s, 3H), 3.82 (d, J = 12.7 Hz, 2H), 3.55- 3.51 (m, 1H),3.05 (t, J = 12.3 Hz, 3H), 2.89 (s, 4H), 2.54 (d, J = 1.0 Hz, 3H), 2.38 (d, J = 12.3 Hz, 2H), 1.96-1.79 (m, 2H), 1.06-0.93 (m, 4H). |
| 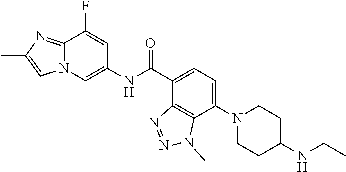 [SMILES: CCNC1CCN(c2ccc(C(=O)Nc3cc(F)c4nc(C)cn4c3)c3nnn(C)c23)CC1] Compound 199 | 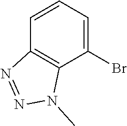 A147 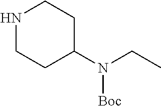 Boc 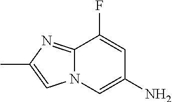 | Modifications: Step 1 was ran using RuPhos and Pd₂(dba)₃. Step 2 was ran for 2 h. Step 3 was ran at 110° C. Step 4 was ran for 2 h. Step 5 was ran for 1 h. The crude product was purified by reversed- phase flash chromatography (Condition 3, Gradient 9) to afford 7-[4-(ethylamino)-1- piperidyl]-N-(8-fluoro-2-methylimidazo[1,2- a]-pyridin-6-yl)-1-methylbenzotriazole-4- carboxamide (Compound 199, 0.02 g, 44.39 μmol). LCMS (ES, m/z): 451 [M + H]⁺. ¹H NMR (300 MHz, DMSO-d₆) δ 10.87 (s, 1H), 9.18 (d, J = 1.7 Hz, 1H), 7.99 (d, J = 7.9 Hz, 1H), 7.93 (dd, J = 3.2, 1.0 Hz, 1H), 7.39 (dd, J = 12.4, 1.7 Hz, 1H), 7.23 (d, J = 7.9 Hz, 1H), 4.57 (s, 3H), 3.38 (d, J = 12.1 Hz, 2H), 2.86 (t, J = 11.0 Hz, 2H), 2.72-2.55 (m, 3H), 2.39-2.33 (m, 3H), 2.01 (d, J = 12.6 Hz, 2H), 1.57 (q, J = 10.3 Hz, 2H), 1.06 (t, J = 7.1 Hz, 3H). |
| 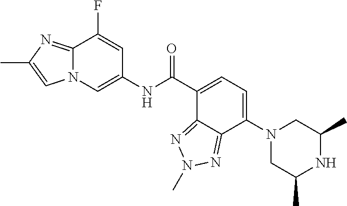 Compound 252 | 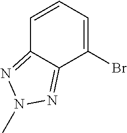 A248 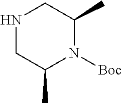 Boc 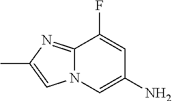 | Modifications: Step 1 was ran for 16 h. Step 6 was ran for 2 h. The crude product was purified by reverse flash chromatography (Condition 4, Gradient 1) to afford 7-((3S,5R)-3,5-dimethyl-piperazin-1- yl)-N-(8-fluoro-2-methyl-imidazo[1,2- a]pyridin-6-yl)-2-methyl-2H- benzo[d][1,2,3]triazole-4-carboxamide 2,2,2- trifluoroacetate (Compound 252, 26.5 mg). LCMS (ES, m/z): 437 [M − TFA + H]⁺. ¹H NMR (400 MHz, DMSO-d₆) δ 10.27 (s, 1H), 9.27 (s, 1H), 9.14 (s, 1H), 8.61 (s, 1H), 8.04 (d, J = 8.3 Hz, 1H), 7.57 (s, 1H), 6.92 (d, J = 8.3 Hz, 1H), 4.72 (d, J = 13.5 Hz, 2H), 4.64 (s, 3H), 3.07 (dd, J = 14.0, 11.2 Hz, 2H), 2.39 (s, 3H), 1.33 (d, J = 6.5 Hz, 6H). |

### Example 29: Synthesis of Compound 157

### Synthesis of Intermediate A133

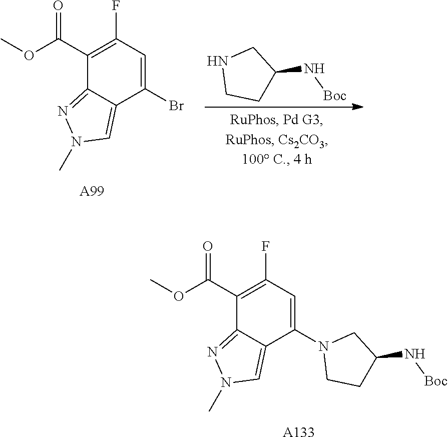

To a stirred mixture of Intermediate A99 (1 g, 3.48 mmol) and tert-butyl N-[(3S)-pyrrolidin-3-yl]carbamate (778.5 mg, 4.18 mmol) in dioxane (20 mL) were added Cs₂CO₃ (3.40 g, 10.5 mmol), RuPhos (325 mg, 696.7 μmol) and RuPhos Pd G3 (291.3 mg, 348.3 μmol) at room temperature. The resulting mixture was stirred for 4 h at 100° C. under N₂, allowed to cool to room temperature, and then concentrated under reduced pressure. The resulting mixture was purified by silica gel column chromatography (25% EtOAc in PE) to afford methyl 4-[(3S)-3-(tert-butoxycarbonylamino)pyrrolidin-1-yl]-6-fluoro-2-methyl-indazole-7-carboxylate (A133, 0.96 g, 2.45 mmol). LCMS (ES, m/z): 393 [M+H]⁺.

### Synthesis of Intermediate A134

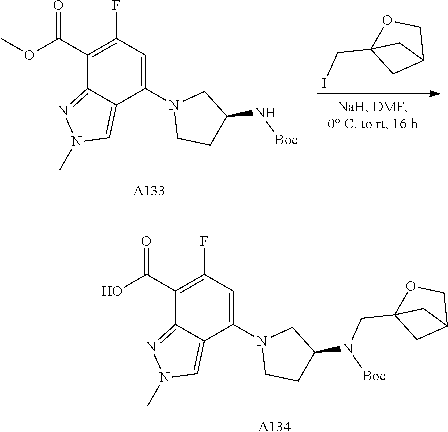 [SMILES: COC(=O)c1c(F)cc(N2CC[C@H](NC(=O)OC(C)(C)C)C2)c2cn(C)nc12 ; ICC12CC(CO1)C2 ; "A133" ; Cn1cc2c(N3CC[C@H](N(CC45CC(CO4)C5)C(=O)OC(C)(C)C)C3)cc(F)c(C(=O)O)c2n1 ; "A134"]

A solution of Intermediate A133 (0.5 g, 1.27 mmol) in DMF (10 mL) was treated with NaH (61.1 mg, 2.55 mmol) for 30 min at 0° C. under N₂ followed by the addition of 1-(iodomethyl)-2-oxabicyclo[2.1.1]hexane (371 mg, 1.66 mmol) in portions at 0° C. The resulting mixture was stirred for 16 h at room temperature, quenched with water at 0° C., and subsequently extracted with EtOAc (3×20 mL). The combined organic layers were washed with water (2×40 mL), brine (40 mL) dried over anhydrous Na₂SO₄, and filtered. The filtrate was concentrated under reduced pressure and purified by trituration with MTBE (10 mL), and the resulting solids were collected by filtration and dried to afford 4-[(3S)-3-[tert-butoxycarbonyl(2-oxabicyclo[2.1.1]hexan-1-ylmethyl)amino]pyrrolidin-1-yl]-6-fluoro-2-methyl-indazole-7-carboxylic acid (A134, 0.25 g, 526.8 μmol). LCMS (ES, m/z): 475 [M+H]⁺.

### Synthesis of Intermediate A135

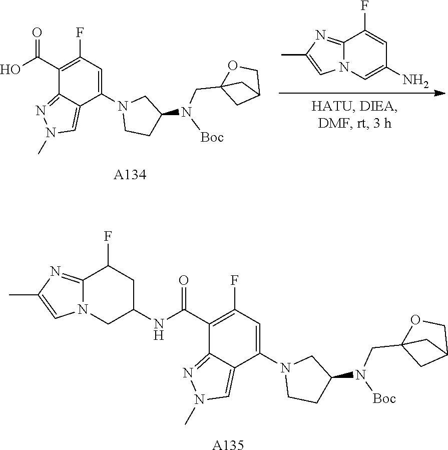

To a stirred mixture of Intermediate A134 (0.25 g, 526.8 μmol) and 8-fluoro-2-methyl-imidazo[1,2-a]pyridin-6-amine (104.4 mg, 632.2 μmol) in DMF (5 mL) were added HATU (300.4 mg, 790.3 μmol) and DIEA (204.2 mg, 1.58 mmol, 275.3 μL) at room temperature. The resulting mixture was stirred for 3 h, diluted with water (20 mL), and subsequently extracted with EtOAc (3×30 mL). The combined organic layers were washed with water (2×80 mL) and brine (80 mL), dried over anhydrous Na₂SO₄, and filtered. The filtrate was concentrated under reduced pressure and purified by silica gel column chromatography (75% EtOAc in PE) to afford tert-butyl N-[(3S)-1-[6-fluoro-7-[(8-fluoro-2-methyl-imidazo[1,2-a]pyridin-6-yl)-carbamoyl]-2-methyl-indazol-4-yl]pyrrolidin-3-yl]-N-(2-oxabicyclo[2.1.1]hexan-1-ylmethyl)-carbamate (A135, 0.28 g, 442.4 μmol). LCMS (ES, m/z): 622 [M+H]⁺.

### Synthesis of Compound 157

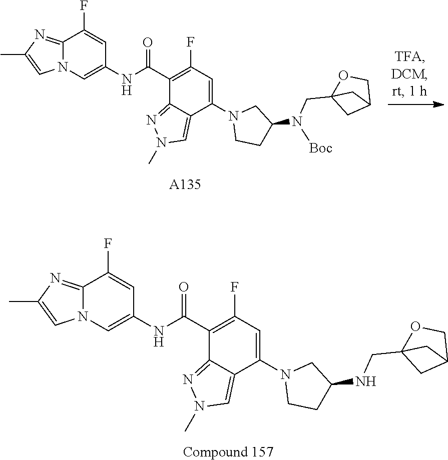

To a stirred mixture of Intermediate A135 (130 mg, 209.1 μmol) in DCM (2 mL) was added TFA (0.5 mL) dropwise, and the reaction was stirred for 1 h at room temperature. The resulting mixture was concentrated under reduced pressure and purified by Prep-HPLC (Condition 5, Gradient 7) to afford 6-fluoro-N-(8-fluoro-2-methyl-imidazo[1,2-a]pyridin-6-yl)-2-methyl-4-[(3S)-3-(2-oxabicyclo[2.1.1]hexan-1-ylmethylamino)pyrrolidin-1-yl]indazole-7-carboxamide (Compound 157, 35 mg, 67.11 μmol). LCMS (ES, m/z): 522 [M+H]⁺. ¹H NMR (400 MHz, DMSO-6) δ 11.03 (s, 1H), 9.18 (s, 1H), 8.80 (s, 1H), 7.87 (d, J=3.1 Hz, 1H), 7.35-7.10 (m, 1H), 5.76 (d, J=16.0 Hz, 1H), 4.22 (s, 3H), 3.78-3.77 (m, 1H), 3.70-3.62 (m, 4H), 3.50 (t, J=5.4 Hz, 1H), 3.43 (t, J=5.8 Hz, 1H), 2.97-2.81 (m, 3H), 2.35 (s, 3H), 2.17 (dq, J=13.0, 6.8 Hz, 1H), 1.99-1.79 (m, 2H), 1.73 (q, J=3.2 Hz, 2H), 1.39-1.31 (m, 2H).

### Example 30: Synthesis of Compound 198

### Synthesis of Intermediate A142

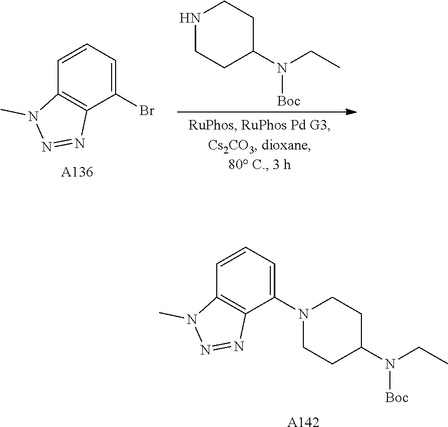

To a stirred mixture of Intermediate A136 (0.28 g, 1.32 mmol) and tert-butyl N-ethyl-N-(4-piperidyl)carbamate (361.8 mg, 1.58 mmol) in dioxane (5 mL) were added Cs₂CO₃ (860.4 mg, 2.64 mmol), RuPhos (123.2 mg, 264.1 μmol) and RuPhos Pd G3 (110.4 mg, 132.1 μmol) at room temperature. The resulting mixture was stirred for 3 h at 80° C. under N₂, allowed to cool to room temperature, diluted with water (10 mL), and subsequently extracted with EtOAc (2×20 mL). The combined organic layers were washed with water (2×40 mL), brine (40 mL), dried over anhydrous Na₂SO₄, and filtered. The filtrate was concentrated under reduced pressure and purified by silica gel column chromatography (50% EtOAc in PE) to afford tert-butyl N-ethyl-N-[1-(1-methylbenzotriazol-4-yl)-4-piperidyl]carbamate (A142, 0.32 g, 890.2 μmol). LCMS (ES, m/z): 360 [M+H]⁺.

### Synthesis of Intermediate A143

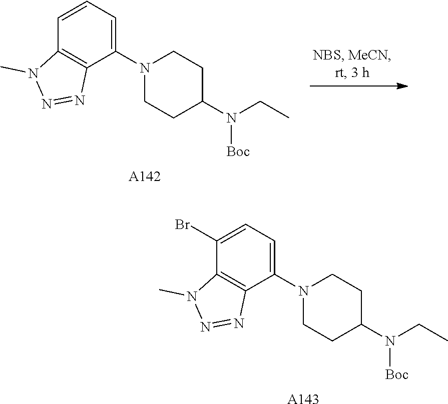

To a stirred mixture of Intermediate A142 (0.32 g, 890.2 μmol) in MeCN (5 mL) were added NBS (158.4 mg, 890.2 μmol). The resulting mixture was stirred for 3 h at room temperature, diluted with water (20 mL), and then extracted with EtOAc (2×20 mL). The combined organic layers were washed with water (2×40 mL), brine (40 mL), dried over anhydrous Na₂SO₄, filtered, and concentrated under reduced pressure to afford tert-butyl (1-(7-bromo-1-methyl-1H-benzo[d][1,2,3]triazol-4-yl)piperidin-4-yl)(ethyl)carbamate (A143, 0.3 g, 684.4 μmol). LCMS (ES, m/z): 438 [M+H]⁺.

### Synthesis of Intermediate A144

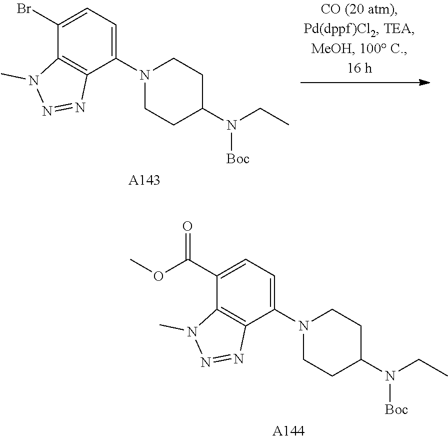

To a solution of Intermediate A143 (0.32 g, 730 μmol) in MeOH (2 mL) were added TEA (369.3 mg, 3.65 mmol, 508.7 μL) and Pd(dppf)Cl₂ (53.4 mg, 73.0 μmol) in a pressure tank. The mixture was purged with nitrogen for 3 h, pressurized to 20 atm with carbon monoxide at 100° C. for 16 h, cooled to room temperature, filtered to remove insoluble solids, diluted with water (10 mL), and extracted with EtOAc (2×20 mL). The combined organic layers were washed with water (2×40 mL), brine (40 mL), dried over anhydrous Na₂SO₄, and filtered. The filtrate was concentrated under reduced pressure and purified by silica gel column chromatography (89% EtOAc in PE) to afford methyl 7-[4-[tert-butoxycarbonyl-(ethyl)amino]-1-piperidyl]-3-methyl-benzotriazole-4-carboxylate (A144, 0.22 g, 527 μmol). LCMS (ES, m/z): 418 [M+H]⁺.

### Synthesis of Intermediate A145

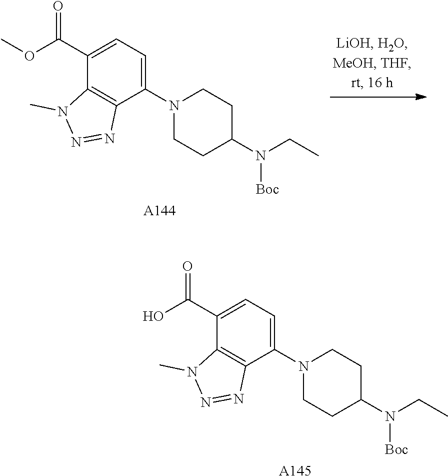 [SMILES: CCN(C(=O)OC(C)(C)C)C1CCN(c2ccc(C(=O)OC)c3c2nnn3C)CC1 ; "A144" ; CCN(C(=O)OC(C)(C)C)C1CCN(c2ccc(C(=O)O)c3c2nnn3C)CC1 ; "A145"]

To a solution of Intermediate A144 (0.22 g, 527.6 μmol) in MeOH (2 mL), H₂O (2 mL) and THF (2 mL) was added LiOH (50.6 mg, 2110.3 μmol). The resulting mixture was stirred for 16 h at room temperature, then neutralized to pH 6 with HCl (0.5 N), and later extracted with EtOAc (3×10 mL). The combined organic layers were washed with water (2×20 mL), brine (20 mL), dried over anhydrous Na₂SO₄, and filtered. The filtrate was concentrated under reduced pressure and purified by silica gel column chromatography (89% EtOAc in PE) to afford 4-(4-((tert-butoxycarbonyl)(ethyl)amino)piperidin-1-yl)-1-methyl-1H-benzo[d][1,2,3]triazole-7-carboxylic acid (A145, 0.20 g, 496.3 μmol). LCMS (ES, m/z): 404 [M+H]⁺.

### Synthesis of Intermediate A146

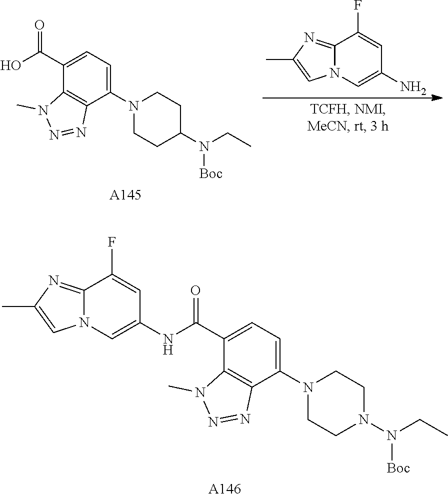 [SMILES: CCN(C(=O)OC(C)(C)C)C1CCN(c2ccc(C(=O)O)c3c2nnn3C)CC1 ; Cc1cn2cc(N)cc(F)c2n1 ; "A145" ; CCN(C(=O)OC(C)(C)C)N1CCN(c2ccc(C(=O)Nc3cc(F)c4nc(C)cn4c3)c3c2nnn3C)CC1 ; "A146"]

To a stirred mixture of Intermediate A145 (0.20 g, 496.3 μmol) and 8-fluoro-2-methyl-imidazo[1,2-a]pyridin-6-amine (98.26 mg, 595.5 μmol) in MeCN (5 mL) were added TCFH (208.6 mg, 744.4 μmol) and NMI (122.0 mg, 1.49 mmol, 118 μL), and the reaction was stirred for 3 h at room temperature. The resulting mixture was diluted with water (20 mL) and extracted with EtOAc (2×20 mL). The combined organic layers were washed with water (2×40 mL), brine (40 mL), dried over anhydrous Na₂SO₄, and filtered. The filtrate was concentrated under reduced pressure and purified by silica gel column chromatography (89% EtOAc in PE) to afford tert-butyl N-ethyl-N-[1-[7-[(8-fluoro-2-methyl-imidazo[1,2-a]pyridin-6-yl)carbamoyl]-1-methyl-benzotriazol-4-yl]-4-piperidyl]carbamate (A146, 0.15 g, 272.4 μmol). LCMS (ES, m/z): 551 [M+H]⁺.

### Synthesis of Compound 198

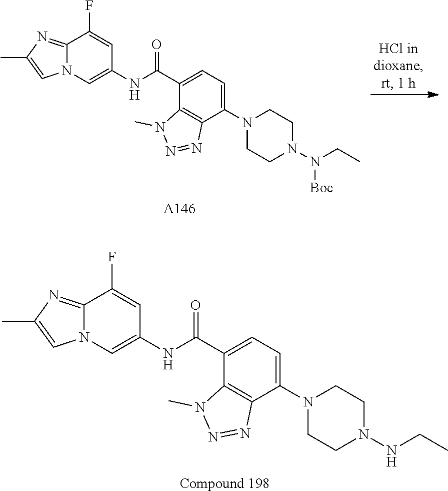

To a solution of Intermediate A146 (0.1 g, 181.6 μmol) in DCM (3 mL) was added TFA (1 mL). The reaction was stirred for 1 h at room temperature, then concentrated under reduced pressure, and adjusted to pH 7 with saturated NaHCO₃ (aq.). The resulting mixture was diluted with water (5 mL) and extracted with DCM/MeOH (20/1) (2×10 mL). The combined organic layers were washed with water (10 mL), brine (10 mL), dried over anhydrous Na₂SO₄, and filtered. The filtrate was concentrated under reduced pressure and purified by reversed-phase flash chromatography (Condition 6, Gradient 1) to afford 4-(4-(ethylamino)piperidin-1-yl)-N-(8-fluoro-2-methylimidazo[1,2-a]pyridin-6-yl)-1-methyl-1H-benzo[d][1,2,3]triazole-7-carboxamide 2,2,2-trifluoroacetate (Compound 198, 0.01 g, 17.71 μmol). LCMS (ES, m/z): 451 [M+H]⁺. ¹H NMR (400 MHz, DMSO-d₆) δ 10.70 (s, 1H), 9.26 (s, 1H), 8.46 (s, 2H), 8.09 (s, 1H), 7.79 (d, J=8.2 Hz, 1H), 7.51 (d, J=12.4 Hz, 1H), 6.76 (d, J=8.3 Hz, 1H), 4.83 (d, J=13.2 Hz, 2H), 4.33 (s, 3H), 3.44 (s, 1H), 3.17 (t, J=12.6 Hz, 2H), 3.05 (q, J=6.6 Hz, 2H), 2.41 (s, 3H), 2.16 (d, J=12.4 Hz, 2H), 1.67 (td, J=13.0, 12.6, 9.2 Hz, 2H), 1.22 (t, J=7.2 Hz, 3H).

An analogous method was followed to obtain the following compounds.

| | Starting | |
|---|---|---|
| Compound | Material | Characterization |
| 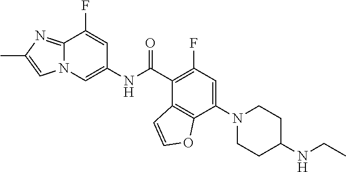 [SMILES: CCNC1CCN(c2cc(F)c(C(=O)Nc3cc(F)c4nc(C)cn4c3)c3ccoc23)CC1] Compound 195 | 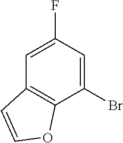 [SMILES: Fc1cc(Br)c2occc2c1] A121 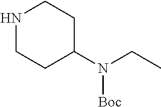 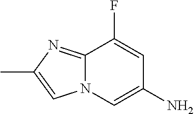 | Modifications: Step 1 was ran using BINAP (0.1 eq) and Pd₂(dba)₃ (0.05 eq) in toluene at 120° C. for 2 h. Step 2 was ran for 2 h in DCM. Step 3 was ran at 130° C. Step 4 was ran at 50° C. for 24 h. Step 5 was ran using HATU (1.3 eq), and DIEA (5 eq) in DMF at room temperature for 5 h. Step 6 was ran for 2 h. The crude product was purified by Prep-HPLC (Condition 5, Gradient 6) to afford 7-[4-(ethylamino)-1- piperidyl]-5-fluoro-N-(8-fluoro-2- methylimidazo[1,2-a]pyridin-6-yl)- benzofuran-4-carboxamide (Compound 195, 28 mg, 61.7 μmol). LCMS (ES, m/z): 454 [M + H]⁺. ¹H NMR (400 MHz, DMSO-d₆) δ 10.20 (d, J = 2.1 Hz, 1H), 9.13 (s, 1H), 8.09 (d, J = 2.1 Hz, 1H), 7.92 (d, J = 3.1 Hz, 1H), 7.24 (d, J = 12.3 Hz, 1H), 7.13 (d, J = 2.1 Hz, 1H), 6.71 (d, J = 13.9 Hz, 1H), 3.98 (d, J = 12.8 Hz, 2H), 3.03 (t, |
| | | J = 11.7 Hz, 2H), 2.61 (q, J = 7.1 Hz, |
| | | 4H), 2.35 (s, 3H), 1.95 (d, J = 12.8 Hz, |
| | | 2H), 1.44-1.34 (m, 2H), 1.04 (t, J = |
| | | 7.1 Hz, 3H). |
| 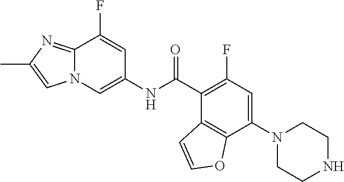 [SMILES: Cc1cn2cc(NC(=O)c3c(F)cc(N4CCNCC4)c4occc34)cc(F)c2n1] Compound 247 | 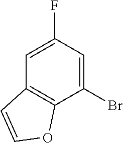 [SMILES: Fc1cc(Br)c2occc2c1] A237 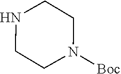 [SMILES: CC(C)(C)OC(=O)N1CCNCC1] 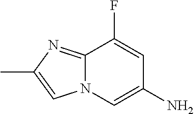 | Modifications: Step 1 was ran using BINAP (0.1 eq) and Pd₂(dba)₃ (0.05 eq) in toluene at 120° C. for 2 h. Step 2 was ran in DCM for 2 h. Step 3 was ran at 130° C. Step 4 was ran at 50° C. for 20 h. Step 5 was ran using HATU (1.3 eq), and DIEA (5 eq) in DMF at room temperature for 5 h. Step 6 was ran for 2 h. The resulting mixture was concentrated under reduced pressure and purified by Prep-HPLC (Condition 3, Gradient 1) to afford 5- fluoro-N-(8-fluoro-2-methylimidazo- [1,2-a]pyridin-6-yl)-7-piperazin-1-yl- benzofuran-4-carboxamide (Compound 247, 26 mg, 63.2 μmol). LCMS (ES, m/z): 412 [M + H]⁺. ¹H NMR (300 MHz, DMSO-d₆) δ 10.22 (s, 1H), 9.13 (s, 1H), 8.09 (d, J = 2.1 Hz, 1H), 7.92 (d, J = 3.1 Hz, 1H), 7.24 |
| | | (d, J = 11.9 Hz, 1H), 7.13 (d, J = 2.1 Hz, |
| | | 1H), 6.70 (d, J = 13.9 Hz, 1H), |
| | | 3.36 (s, 4H), 2.90 (t, J = 5.0 Hz, 4H), |
| | | 2.35 (d, J = 0.8 Hz, 3H). |
| 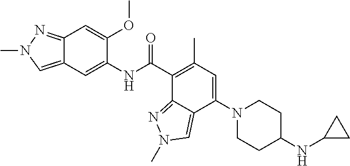 Compound 436 | 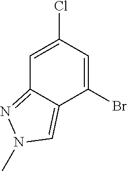 B170 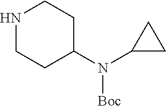 [SMILES: CC(C)(C)OC(=O)N(C1CCNCC1)C1CC1] 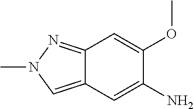 | Modifications: Step 1 was ran for 16 h. Step 2 was ran using NIS in DMF for 2 h. Step 3 was ran at 80° C. Step 4 was ran at 60° C. for 12 h. Step 5 was ran for 2 h. Step 6 was ran using TFA in DCM for 2 h. The resulting mixture was purified by reversed-phase flash chromatography (Condition 3, Gradient 22) to afford 6- chloro-4-[4-(cyclopropylamino)-1- piperidyl]-N-(6-methoxy-2-methyl- indazol-5-yl)-2-methyl-indazole-7- carboxamide (Compound 436, 45.8 mg, 90.2 μmol, 50% yield) as a solid. LCMS (ES, m/z): 508 [M + H]⁺. ¹H NMR (400 MHz, DMSO-d₆) δ 11.15 (s, 1H), 8.76 (d, J = 5.7 Hz, 2H), 8.18 (s, 1H), 7.06 (s, 1H), 6.35 (s, 1H), 4.23 (s, 3H), 4.09 (s, 3H), 3.99 (s, 3H), 3.84 (d, J = 12.7 Hz, 2H), 3.10-3.00 (m, 2H), 2.77 (dt, J = 9.9, 5.7 Hz, 1H), 2.31 (s, 1H), 2.12 (tt, J = 6.7, 3.5 Hz, 1H), 2.04-1.96 (m, 2H), 1.53-1.40 (m, 2H), 0.40 (td, J = 6.3, 4.0 Hz, 2H), 0.28-0.20 (m, 2H). |
| 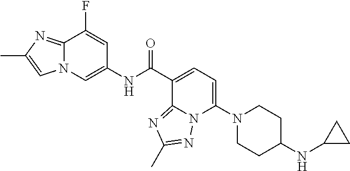 [SMILES: Cc1cn2cc(NC(=O)c3ccc(N4CCC(NC5CC5)CC4)n4nc(C)nc34)cc(F)c2n1] Compound 640 | 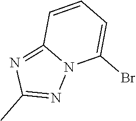 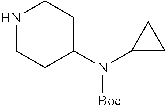 [SMILES: CC(C)(C)OC(=O)N(C1CCNCC1)C1CC1] 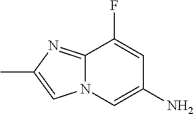 | Modification: Step 1 was ran at 90° C.f or 16 h. Step 3 was ran at 110° C. for 16 h. Step 5 was ran for 2 h. Step 6 was ran for 2 h. The crude product was purified by Prep-HPLC (Condition 5, Gradient 12) to afford 5-[4- (cyclopropylamino)-1-piperidyl]-N- (8-fluoro-2-methyl-imidazo[1,2- a]pyridin-6-yl)-2-methyl-[1,2,4]- triazolo[1,5-a]-pyridine-8- carboxamide (Compound 640, 19.9 mg, 43 μmol, 35% yield) as a solid. LCMS (ES, m/z): 463 [M + H]⁺. ¹H NMR (400 MHz, DMSO-d₆) δ 11.49 (s, 1H), 9.14 (d, J = 1.7 Hz, 1H), 8.25 (d, J = 8.4 Hz, 1H), 7.92 (d, J = 3.4 Hz, 1H), 7.33 (dd, J = 12.3, 1.7 Hz, 1H), 6.70 (d, J = 8.5 Hz, 1H), 4.20 (d, J = 12.5 Hz, 2H), 3.25-3.15 (m, 2H), 2.83 (dt, J = 9.8, 5.7 Hz, 1H), 2.63 (s, 3H), 2.35 (s, 3H), 2.13 (tt, J = 6.7, 3.6 Hz, |
| | | 1H), 2.02 (d, J = 12.8 Hz, 2H), 1.50 (q, |
| | | J = 9.8 Hz, 2H), 0.40 (dt, J = 6.2, |
| | | 3.0 Hz, 2H), 0.28-0.20 (m, 2H). |
| 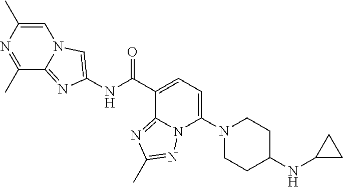 Compound 641 | 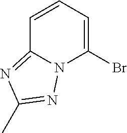 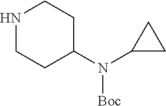 [SMILES: CC(C)(C)OC(=O)N(C1CCNCC1)C1CC1] 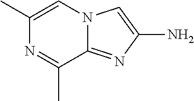 | Modification: Step 1 was ran at 90° C. for 16 h. Step 3 was ran at 110° C. for 16 h. Step 5 was ran for 2 h. Step 6 was ran using HCl in dioxane and DCM at room temperature for 2 h. The resulting mixture was purified by Prep-HPLC (Condition 5, Gradient 13) to afford 5-[4- (cyclopropylamino)-1-piperidyl]-N- (6,8-dimethylimidazo-[1,2-a]pyrazin- 2-yl)-2-methyl-[1,2,4]-triazolo[1,5- a]pyridine-8-carboxamide (Compound 641, 9.5 mg, 20.7 μmol, 19% yield) as a solid. LCMS (ES, m/z): 560 [M + H]⁺. ¹H NMR (400 MHz, DMSO-d₆) δ 11.90 (s, 1H), 8.36-8.28 (m, 3H), 6.73 (d, J = 8.5 Hz, 1H), 4.22 (d, J = 12.5 Hz, 2H), 3.22 (t, J = 11.6 Hz, 2H), 2.83 (s, 1H), 2.71 (s, 3H), 2.65 (s, 3H), 2.38 (s, 3H), 2.17- 2.10 (m, 1H), 2.03 (d, J = 12.6 Hz, 2H), 1.51 (q, J = 10.5, 9.7 Hz, 2H), |
| | | 0.40 (dt, J = 6.3, 3.0 Hz, 2H), 0.27- |
| | | 0.21 (m, 2H). |
| 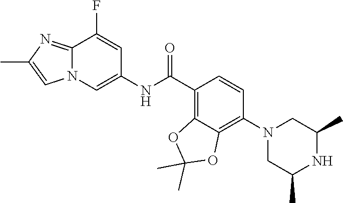 Compound 656 | 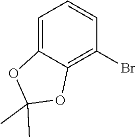 B130 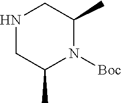 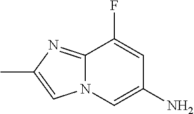 | Modifications: Step 1 was ran for 16 h at 100° C. Step 2 was ran using NIS for 16 h. Step 3 was ran at 60° C. Step 4 was ran at 60° C. for 24 h. Step 5 was ran using HATU (1.3 eq), and DIEA (5 eq) in DMF at 60° C. for 16 h. Step 6 was ran using TFA in DCM for 2 h. The crude product was purified by reversed-phase flash chromatography (Condition 3, Gradient 30) to afford 7- [(3S,5R)-3,5-dimethylpiperazin-1- yl]-N-(8-fluoro-2-methyl- imidazo[1,2-a]-pyridin-6-yl)-2,2- dimethyl-1,3-benzodioxole-4- carboxamide (Compound 656, 40 mg, 88.2 μmol, 42% yield) as a solid. LCMS (ES, m/z): 454 [M + H]⁺. ¹H NMR (400 MHz, DMSO-d₆) δ 9.39 (s, 1H), 9.06 (d, J = 1.7 Hz, 1H), 7.88 (d, J = 3.1 Hz, 1H), 7.29 (dd, J = 12.6, 1.7 Hz, 1H), 7.25 (d, J = 8.8 Hz, 1H), 6.55 (d, J = 8.9 Hz, 1H), 3.59 (dd, J = 11.8, 2.6 Hz, 2H), 2.92-2.81 (m, 2H), 2.34 (s, 3H), 2.25 (t, J = 11.0 Hz, |
| | | 2H), 1.75 (s, 6H), 1.01 (d, J = 6.2 Hz, |
| | | 6H). |
| 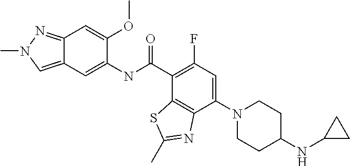 [SMILES: COc1cc2nn(C)cc2cc1NC(=O)c1c(F)cc(N2CCC(NC3CC3)CC2)c2nc(C)sc12] Compound 661 | 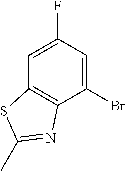 D46 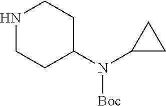 [SMILES: CC(C)(C)OC(=O)N(C1CCNCC1)C1CC1] 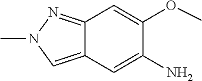 | Modification: Step 1 was ran at 90° C. for 6 h. Step 3 was ran at 130° C. for 24 h. Step 4 was ran for 6 h. Step 6 was ran using TFA in DCM at room temperature for 2 h. The mixture was purified by reversed- phase flash chromatography (Condition 3, Gradient 18) to afford 4- [4-(cyclopropyl-amino)-1-piperidyl]- 6-fluoro-N-(6-methoxy-2-methyl- indazol-5-yl)-2-methyl-1,3- benzothiazole-7-carboxamide (Compound 661, 0.05 g, 98.3 μmol). LCMS (ES, m/z): 509 [M + H]⁺. ¹H NMR (400 MHz, DMSO-d₆) δ 9.54 (s, 1H), 8.67 (s, 1H), 8.23 (s, 1H), 7.11 (s, 1H), 6.89 (d, J = 16.7 Hz, 1H), 4.39 (d, J = 12.6 Hz, 2H), 4.10 (s, 3H), 3.98 (s, 3H), 3.16 (dd, J = 13.1, 10.4 Hz, 2H), 2.77 (s, 4H), 2.24 (s, 1H), 1.98 (d, J = 12.3 Hz, 2H), 1.45 (q, J = 10.8, 10.0 Hz, 2H), 1.24 (s, 0.5H), 0.40 (td, J = 6.3, 4.0 Hz,2H), 0.26-0.20 (m, 2H). |
| 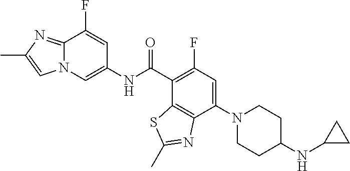 [SMILES: Cc1cn2cc(NC(=O)c3c(F)cc(N4CCC(NC5CC5)CC4)c4nc(C)sc34)cc(F)c2n1] Compound 662 | 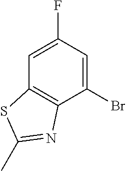 D46 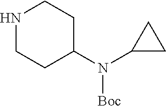 [SMILES: CC(C)(C)OC(=O)N(C1CCNCC1)C1CC1] 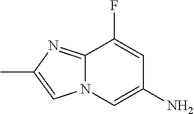 | Modification: Step 1 was ran at 90° C. for 6 h. Step 3 was ran at 130° C. for 24 h. Step 4 was ran for 6 h. Step 6 was ran using TFA in DCM at room temperature for 2 h. The mixture was purified by reversed- phase flash chromatography (Condition 3, Gradient 37) to afford 4- [4-(cyclopropyl-amino)-1-piperidyl]- 6-fluoro-N-(8-fluoro-2-methyl- imidazo[1,2-a]pyridin-6-yl)-2- methyl-1,3-benzothiazole-7- carboxamide (Compound 662, 0.025 g, 50.3 μmol). LCMS (ES, m/z): 497 [M + H]⁺. ¹H NMR (400 MHz, DMSO-d₆) δ 9.90 (s, 1H), 9.07 (d, J = 1.6 Hz, 1H), 7.93 (dd, J = 3.2, 1.0 Hz, 1H), 7.31 (dd, J = 12.6, 1.7 Hz, 1H), 6.86 (d, J = 15.1 Hz, 1H), 4.35 (d, J = 12.5 Hz, 2H), 3.19-3.08 (m, 2H), 2.76 (s, 4H), 2.35 (d, J = 0.9 Hz, 3H), 2.22 (s, 1H), 2.13 (tt, J = 6.7, 3.6 Hz, 1H), 1.97 (d, J = 12.6 Hz, 2H), 1.47 (d, J = 13.2 Hz, 2H), 1.43 (s, 1H), 0.44-0.35 (m, 2H), 0.30-0.19 (m, 2H). |
| 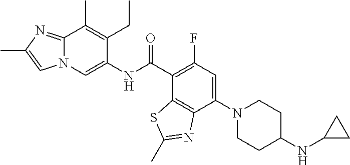 [SMILES: CCc1c(NC(=O)c2c(F)cc(N3CCC(NC4CC4)CC3)c3nc(C)sc23)cn2cc(C)nc2c1C] Compound 663 | 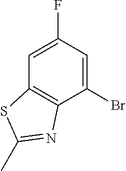 D46 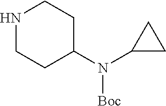 [SMILES: CC(C)(C)OC(=O)N(C1CCNCC1)C1CC1] 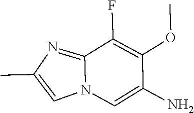 | Modification: Step 1 was ran at 90° C. for 6 h. Step 3 was ran at 130° C. for 24 h. Step 4 was ran for 6 h. Step 6 was ran using TFA in DCM at room temperature for 2 h. The crude product was purified by Prep-HPLC (Condition 6, Gradient 10) to afford 4-[4- (cyclopropylamino)-1-piperidyl]-6- fluoro-N-(8-fluoro-7-methoxy-2- methyl-imidazo[1,2-a]-pyridin-6-yl)- 2-methyl-1,3-benzothiazole-7- carboxamide (Compound 663, 9.8 mg, 18.6 μmol) LCMS (ES, m/z): 527 [M + H]⁺. ¹H NMR (400 MHz, DMSO-d₆) δ 9.32 (d, J = 16.0 Hz, 1H), 9.22 (d, J = 1.2 Hz, 1H), 7.82 (dd, J = 3.2, 1.1 Hz, 1H), 6.90 (d, J = 16.6 Hz, 1H), 4.40 (d, J = 12.8 Hz, 2H), 4.10 (d, J = 2.3 Hz, 3H), 3.17 (t, J = 11.5 Hz, 2H), 2.76-2.75 (m, 1H), 2.75 (s, 3H), 2.31 (d, J = 0.9 Hz, 3H), 2.12 (tt, J = 6.7, 3.6 Hz, 1H), 1.97 (d, J = 12.5 Hz, 2H), 1.45-1.42 (m, 1H), 0.39 (td, J = 6.3, 4.1 Hz, 2H), 0.30-0.18 (m, 2H). |

### Example 31: Synthesis of A156

### Synthesis of Intermediate A152

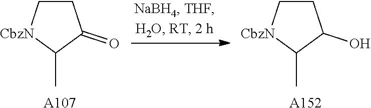

To a stirred mixture of Intermediate A107 (2 g, 8.57 mmol) in THF (20 mL) and H₂O (0.2 mL) was added NaBH₄ (356 mg, 9.43 mmol) in portions at 0° C. The resulting mixture was stirred for 2 h at room temperature, quenched with water (20 ml) at 0° C., and subsequently extracted with DCM (3×100 mL). The combined organic layers were dried over anhydrous Na₂SO₄ and filtered. The filtrate was concentrated under reduced pressure and purified by silica gel column chromatography (17% EtOAc in PE) to afford benzyl 3-hydroxy-2-methyl-pyrrolidine-1-carboxylate (A152, 1.5 g, 6.38 mmol). LCMS (ES, m/z): 236 [M+H]⁺.

### Synthesis of Intermediate A153

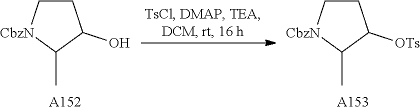

To a stirred solution of Intermediate A152 (1.3 g, 6.07 mmol) and TEA (1.84 g, 18.2 mmol) in DCM (26 mL) was added benzyl carbonochloridate (1.09 g, 6.37 mmol) dropwise at 0° C. The resulting mixture was stirred for 16 h at room temperature, diluted with DCM (100 mL), washed with citric acid solution (100 mL), saturated NaHCO₃ solution (100 mL), and brine (100 mL), then dried over anhydrous Na₂SO₄, and filtered. The filtrate was concentrated under reduced pressure and purified by silica gel column chromatography (9% THF in PE) to afford benzyl 2-methyl-3-(p-tolylsulfonyloxy) pyrrolidine-1-carboxylate (A153, 920 mg, crude). LCMS (ES, m/z): 390 [M+H]⁺.

### Synthesis of Intermediate A154

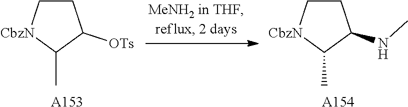

A solution of Intermediate A153 (920 mg, 2.36 mmol) in MeNH₂ in THE solution (10 mL, 2 M) was stirred for 2 days at reflux. The resulting mixture was concentrated under reduced pressure, purified by silica gel column chromatography (25% THF in PE) to afford benzyl (2S,3R)-2-methyl-3-(methylamino)pyrrolidine-1-carboxylate (A154, 235 mg, 946.4 μmol). LCMS (ES, m/z): 249 [M+H]⁺.

### Synthesis of Intermediate A155

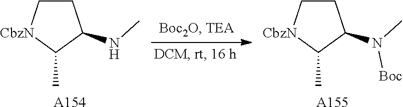

To a stirred solution of Intermediate A154 (225 mg, 906.1 μmol) in DCM (4 mL) were added TEA (275 mg, 2.72 mmol, 378.9 μL) and Boc₂O (395 mg, 1.81 mmol, 415.9 μL), and the reaction was stirred for 16 h at room temperature. The resulting mixture was concentrated under reduced pressure and purified by silica gel column chromatography (9% THF in PE) to afford benzyl (2S,3R)-3-[tert-butoxycarbonyl(methyl)amino]-2-methyl-pyrrolidine-1-carboxylate (A155, 290 mg, 832.3 μmol). LCMS (ES, m/z): 349 [M+H]⁺.

### Synthesis of Intermediate A156

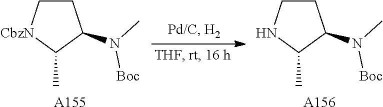

To a solution of Intermediate A155 (280 mg, 803.6 μmol) in THF (15 mL) was added Pd/C (56 mg, 52.6 μmol, 10% purity) in a 25 mL round-bottom flask. The mixture was hydrogenated at room temperature for 16 h under hydrogen atmosphere using a hydrogen balloon, filtered through a filter paper and concentrated under reduced pressure to afford tert-butyl N-methyl-N-[(2S,3R)-2-methylpyrrolidin-3-yl]carbamate (A156, 170 mg, 713.9 μmol) as an oil. LCMS (ES, m/z): 215 [M+H]⁺.

### Example 32: Synthesis of Compound 212

### Synthesis of Intermediate A174

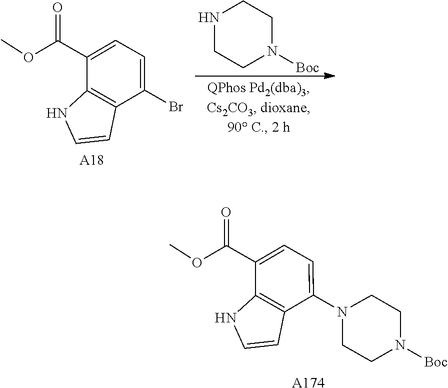

To a solution of methyl 4-bromo-1H-indole-7-carboxylate (A18, 1.1 g, 4.3 mmol) and tert-butyl piperazine-1-carboxylate (1.21 g, 6.5 mmol) in dioxane (20 mL) were added Pd₂(dba)₃ (396 mg, 432.9 μmol), Qphos (616 mg, 865.9 μmol) and Cs₂CO₃ (4.23 g, 12.99 mmol), and the reaction was stirred for 2 h at 90° C. under N₂. The resulting mixture was concentrated under reduced pressure and purified by silica gel column chromatography (50% EtOAc in PE) to afford methyl 4-(4-tert-butoxycarbonylpiperazin-1-yl)-1H-indole-7-carboxylate (A174, 700 mg, 1.95 mmol, 45% yield) as a solid. LCMS (ES, m/z): 360 [M+H]⁺.

### Synthesis of Intermediate A175

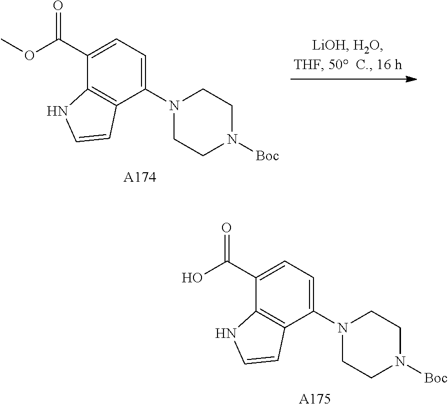

To a stirred mixture of Intermediate A174 (700 mg, 1.95 mmol) in THF (15 mL) and H₂O (2 mL) was added LiOH (233 mg, 9.74 mmol) at room temperature. The resulting mixture was stirred for 16 h at 50° C. and subsequently concentrated under reduced pressure. The mixture residue was neutralized to pH 5 with 1 N of HCl (aq.). The resulting solids were collected by filtration, washed with water (20 mL), and dried to afford 4-(4-tert-butoxycarbonylpiperazin-1-yl)-1H-indole-7-carboxylic acid (A175, 600 mg, 1.74 mmol). LCMS (ES, m/z): 346 [M+H]⁺.

### Synthesis of Intermediate A176

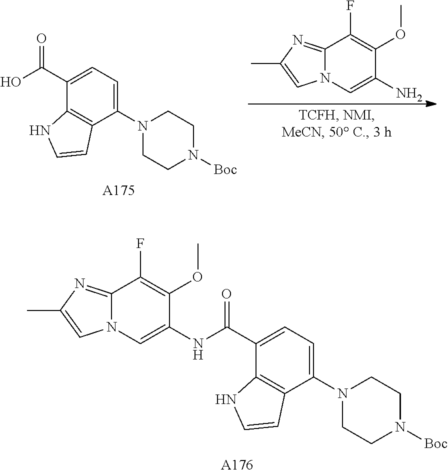

To a stirred solution of Intermediate A175 (60 mg, 173.7 μmol) and 8-fluoro-7-methoxy-2-methyl-imidazo[1,2-a]pyridin-6-amine (40 mg, 208.5 μmol) in MeCN (5 mL) were added NMI (42 mg, 521.2 μmol, 41.34 μL) and TCFH (73 mg, 260.6 μmol), and the reaction was stirred for 3 h at 50° C. The resulting mixture was allowed to cool down to room temperature, concentrated under reduced pressure, and purified by silica gel column chromatography (60% EtOAc in PE) to afford tert-butyl 4-[7-[(8-fluoro-7-methoxy-2-methyl-imidazo[1,2-a]pyridin-6-yl)carbamoyl]-1H-indol-4-yl]piperazine-1-carboxylate (A176, 35 mg, 67 μmol). LCMS (ES, m/z): 523 [M+H]⁺.

### Synthesis of Compound 212

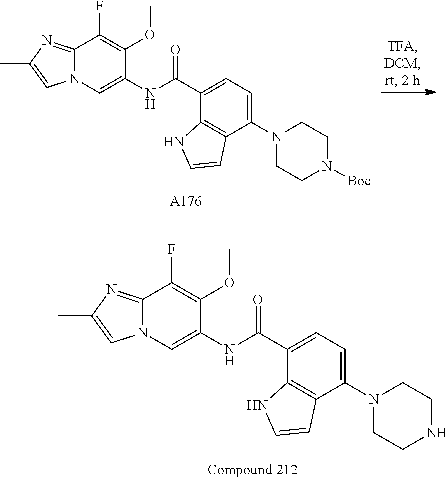

To a stirred solution of Intermediate A176 (35 mg, 66.98 μmol) in DCM (2 mL) was added TFA (0.5 mL), and the reaction was stirred for 2 h at room temperature. The resulting mixture was concentrated under reduced pressure and purified by reversed-phase flash chromatography (Condition 1, Gradient 3) to afford N-(8-fluoro-7-methoxy-2-methyl-imidazo-[1,2-a]pyridin-6-yl)-4-piperazin-1-yl-1H-indole-7-carboxamide (Compound 212, 10 mg, 23.7 μmol). LCMS (ES, m/z): 423 [M+H]⁺. ¹H NMR (400 MHz, DMSO-d₆) δ 8.64 (d, J=1.3 Hz, 1H), 7.77 (d, J=8.1 Hz, 1H), 7.70 (d, J=3.0 Hz, 1H), 7.28 (d, J=3.3 Hz, 1H), 6.56 (d, J=8.2 Hz, 1H), 6.50 (d, J=3.3 Hz, 1H), 4.00 (d, J=2.3 Hz, 3H), 3.25 (t, J=4.8 Hz, 4H), 2.95 (t, J=4.9 Hz, 4H), 2.32 (s, 3H).

An analogous method was followed to obtain the following compounds.

| | Starting | |
|---|---|---|
| Compound | Material | Characterization |
| 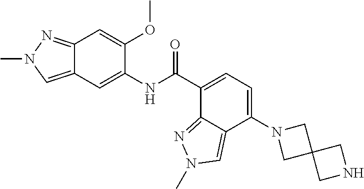 Compound 455 | 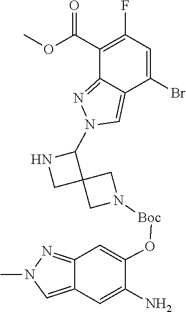 | Modification: Step 1 was ran for 3 h. Step 3 was ran using HATU, DIEA, and DMF at rt. Step 4 was ran for 1 h. The crude product was purified by Prep-HPLC (Condition 2, Gradient 13) to afford 4-(2,6-diazaspiro[3.3]-heptan-2-yl)- 6-fluoro-N-(6-methoxy-2-methyl- indazol-5-yl)-2-methyl-indazole-7- carboxamide (Compound 455, 13.3 mg, 29.6 μmol, 20% yield) as a solid. LCMS (ES, m/z): 450 [M + H]⁺. ¹H NMR (400 MHz, DMSO-d₆) δ 11.49 (s, 1H), 8.77 (s, 1H), 8.66 (s, 1H), 8.58 (s, 1H), 8.16 (s, 1H), 7.05 (s, 1H), 5.73 (d, J = 14.9 Hz, 1H), 4.42 (s, 4H), 4.24 (d, J = 10.2 Hz, 7H), 4.08 (s, 3H), 4.03 (s, 3H). |

### Example 33: Synthesis of Compound 213

### Synthesis of Intermediate A177

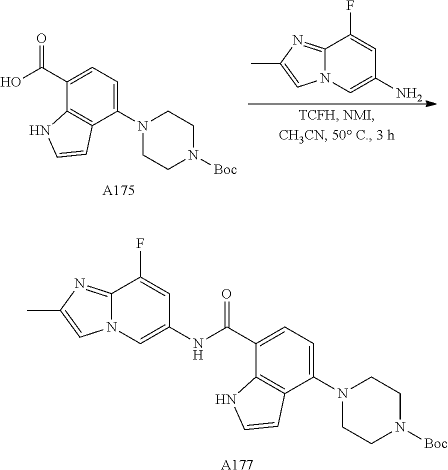

To a stirred solution of Intermediate A175 (60 mg, 173.7 μmol) and 8-fluoro-2-methyl-imidazo[1,2-a]pyridin-6-amine (34 mg, 208.5 μmol) in MeCN (6 mL) were added NMI (42 mg, 521.2 μmol, 41.34 μL) and TCFH (73 mg, 260.6 μmol), and the reaction was stirred for 3 h at 50° C. The resulting mixture was allowed to cool to room temperature, concentrated under reduced pressure, and purified by silica gel column chromatography (67% EtOAc in PE) to afford tert-butyl 4-[7-[(8-fluoro-2-methyl-imidazo[1,2-a]pyridin-6-yl)-carbamoyl]-1H-indol-4-yl]piperazine-1-carboxylate (A177, 35 mg, 71.1 μmol). LCMS (ES, m/z): 493 [M+H]⁺.

### Synthesis of Compound 213

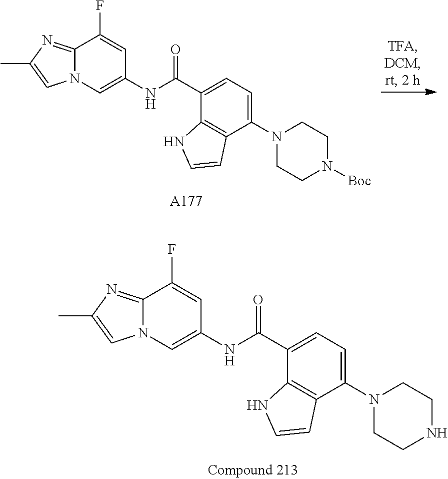

To a stirred solution of Intermediate A177 (35 mg, 71.06 μmol) in DCM (3 mL) was added TFA (0.5 mL), and the reaction was stirred for 2 h at room temperature. The resulting mixture was concentrated under reduced pressure and purified by reversed-phase flash chromatography (Condition 1, Gradient 3) to afford N-(8-fluoro-2-methyl-imidazo[1,2-a]-pyridin-6-yl)-4-piperazin-1-yl-1H-indole-7-carboxamide (Compound 213, 10 mg, 25.5 μmol). LCMS (ES, m/z): 393 [M+H]⁺. ¹H NMR (400 MHz, DMSO-d₆) δ 9.05 (d, J=1.6 Hz, 1H), 7.88 (d, J=3.1 Hz, 1H), 7.80 (d, J=8.2 Hz, 1H), 7.40 (dd, J=12.8, 1.7 Hz, 1H), 7.30 (d, J=3.2 Hz, 1H), 6.58 (d, J=8.2 Hz, 1H), 6.51 (d, J=3.2 Hz, 1H), 3.31-3.24 (m, 4H), 3.01 (t, J=4.8 Hz, 4H), 2.35 (s, 3H).

An analogous method was followed to obtain the following compound.

| Compound | Starting Material | Characterization |
|---|---|---|
| 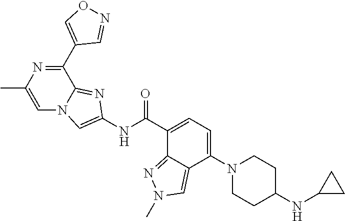 [SMILES: Cc1cn2cc(NC(=O)c3ccc(N4CCC(NC5CC5)CC4)c4cn(C)nc34)nc2c(-c2cnoc2)n1] Compound 462 | 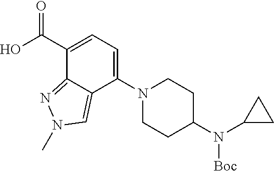 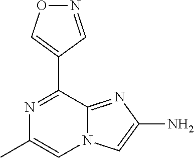 C180 | Modification: Step 1 was ran for 6 h at room temperature. The residue was purified by reversed-phase flash chromatography (Condition 3, Gradient 20) to afford 4-[4- (cyclopropylamino)-1- piperidyl]-N-(8-isoxazol-4-yl-6- methyl-imidazo[1,2-a]-pyrazin- 2-yl)-2-methyl-indazole-7- carboxamide (Compound 462, 10 mg, 19.6 μmol, 24% yield) as a solid. LCMS (ES, m/z): 512 [M + H]⁺. ¹H NMR (400 MHz, DMSO-d₆) δ 11.70 (s, 1H), 9.96 (s, 1H), 9.39 (s, 1H), 8.81 (s, 1H), 8.51-8.44 (m, 2H), 8.03 (d, J = 8.1 Hz, 1H), 6.51 (d, J = 8.2 Hz, 1H), 4.35 (s, 3H), 3.91 (d, J = 12.8 Hz, 2H), 3.09 (t, J = 11.4 Hz, 2H), 2.79 (dt, J = 10.0, 5.9 Hz, |
| | | 1H), 2.51-2.47 (m, 3H), 2.18- |
| | | 2.06 (m, 1H), 2.02 (d, J = 12.3 Hz, |
| | | 2H), 1.48 (q, J = 9.8 Hz, 2H), |
| | | 0.45-0.36 (m, 2H), 0.28-0.20 (m, |
| | | 2H). |
| 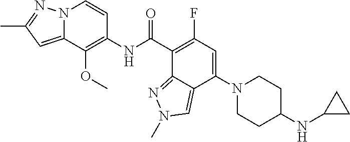 [SMILES: COc1c(NC(=O)c2c(F)cc(N3CCC(NC4CC4)CC3)c3cn(C)nc23)ccn2nc(C)cc12] Compound 447 | 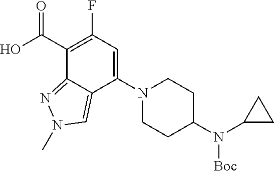 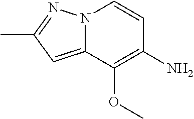 C211 | Modification: Step 1 was ran for 2 h at room temperature. Step 2 was ran for 2 h at room temperature. The crude product was purified by Prep-HPLC (Condition 2, Gradient 9) to afford 4-(4- (cyclopropylamino)piperidin-1- yl)-6-fluoro-N-(4-methoxy-2- methylpyrazolo-[1,5-a]pyridin- 5-yl)-2-methyl-2H-indazole-7- carboxamide 2,2,2- trifluoroacetate (Compound 447, 30 mg, 49.5 μmol, 37% yield) as a solid. LCMS (ES, m/z): 492 [M + H]⁺. ¹H NMR (400 MHz, DMSO-d₆) δ 11.64 (s, 1H), 8.90 (s, 1H), 8.86 (s, 2H), 8.36 (d, J = 7.6 Hz, 1H), 8.09 (d, |
| | | J = 7.6 Hz, 1H), 6.46 (s, 1H), |
| | | 6.34 (d, J = 15.9 Hz, 1H), 4.26 (s, |
| | | 3H), 4.12-4.10 (m, 2H), 4.09 (s, |
| | | 3H), 3.51 (s, 1H), 3.12 (t, J = |
| | | 12.6 Hz, 2H), 2.86-2.71 (m, 1H), |
| | | 2.39 (s, 3H), 2.20 (d, J = 12.3 Hz, |
| | | 2H), 1.72 (td, J = 13.5, 13.0, |
| | | 9.6 Hz, 2H), 0.85 (s, 2H), 0.83 |
| | | (s, 2H). |
| 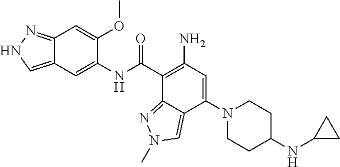 Compound 449 | 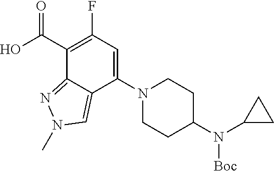 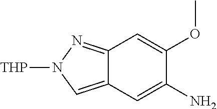 C214 | Modification: Step 1 was ran for 2 h at room temperature. Step 2 was ran for 2 h at room temperature. The residue was purified by reversed-phase flash chromatography (Condition 3, Gradient 21) to afford 4-[4- (cyclopropylamino)-1- piperidyl]-6-fluoro-N-(6- methoxy-2H-indazol-5-yl)-2- methyl-indazole-7-carboxamide (Compound 449, 18.7 mg, 39.2 μmol, 26% yield) as a solid. LCMS (ES, m/z): 478 [M + H]⁺. ¹H NMR (400 MHz, DMSO-d₆) δ 12.79 (s, 1H), 11.55 (s, 1H), 8.87 (s, 1H), 8.78 (s, 1H), 7.94 (s, 1H), 7.07 (s, 1H), 6.21 (d, J = |
| | | 16.1 Hz, 1H), 4.24 (s, 3H), 4.06 |
| | | (s, 3H), 3.89 (d, J = 13.1 Hz, 2H), |
| | | 3.10 (t, J = 11.6 Hz, 2H), 2.79 (d, |
| | | J = 5.3 Hz, 1H), 2.27 (s, 1H), |
| | | 2.11 (tq, J = 8.7, 5.0, 4.3 Hz, 1H), |
| | | 1.99 (d, J = 12.7 Hz, 2H), 1.52- |
| | | 1.38 (m, 2H), 0.40 (dt, J = 6.2, |
| | | 3.1 Hz, 2H), 0.27-0.19 (m, 2H). |
| 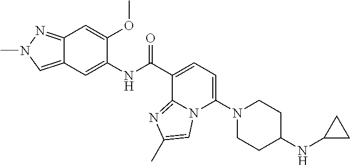 [SMILES: COc1cc2nn(C)cc2cc1NC(=O)c1ccc(N2CCC(NC3CC3)CC2)n2cc(C)nc12] Compound 618 | 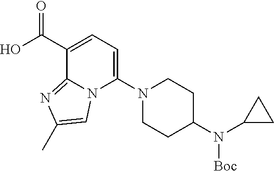 B158 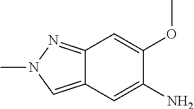 | Modification: Step 1 was ran at room temperature for 3 h. Step 2 was ran using HCI in dioxane and DCM for 2 h at room temperature. The residue was purified by reversed-phase flash chromatography (Condition 3, Gradient 8) to afford 5-[4- (cyclopropylamino)-1- piperidyl]-N-(6-methoxy-2- methyl-indazol-5-yl)-2-methyl- imidazo[1,2-a]-pyridine-8- carboxamide (Compound 618, 0.03 g, 63.4 μmol, 29% yield) as a solid. LCMS (ES, m/z): 474 [M + H]⁺. ¹H NMR (400 MHz, DMSO-d₆) δ 12.79 (s, 1H), 8.81 (s, 1H), 8.18 (s, 1H), 8.08 (d, J = |
| | | 7.9 Hz, 1H), 7.55 (d, J = 1.1 Hz, |
| | | 1H), 7.05 (s, 1H), 6.57 (d, J = |
| | | 7.9 Hz, 1H), 4.07 (s, 3H), 4.01 (s, |
| | | 3H), 3.47 (d, J = 11.8 Hz, 2H), |
| | | 2.92-2.82 (m, 3H), 2.5 (s, 3H), |
| | | 2.11 (dq, J = 6.7, 3.4 Hz, 1H), |
| | | 2.07-1.99 (m, 2H), 1.57 (q, J = |
| | | 11.2 Hz, 2H), 0.39 (dt, J = 6.2, |
| | | 3.0 Hz, 2H), 0.24 (p, J = 3.9 Hz, |
| | | 2H). |
| 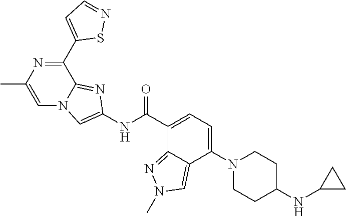 [SMILES: Cc1cn2cc(NC(=O)c3ccc(N4CCC(NC5CC5)CC4)c4cn(C)nc34)nc2c(-c2ccns2)n1] Compound 486 | 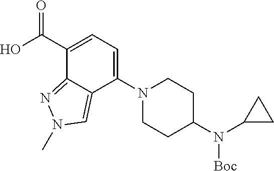 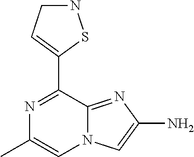 D52 | Modification: Step 2 was ran using TMSOTf and DIEA in DCM for 1 h at room temperature. The resulting mixture was purified by reversed-phase flash chromatography (Condition 3, Gradient 26) to afford 4-(4- (cyclopropylamino)-piperidin-1- yl)-N-(8-(isothiazol-5-yl)-6- methyl-imidazo[1,2-a]-pyrazin- 2-yl)-2-methyl-2H-indazole-7- carboxamide (Compound 486, 11.8 mg). LCMS (ES, m/z): 528 [M + H]⁺. ¹H NMR (400 MHz, DMSO-d₆) δ 11.71 (s, 1H), 8.83 (s, 1H), 8.78 (d, J = 1.8 Hz, 1H), 8.60 (s, 1H), 8.58-8.50 (m, 2H), 8.05 (d, J = 8.2 Hz, 1H), 6.52 (d, J = 8.2 Hz, 1H), 4.35 (s, 3H), 3.92 (d, J = 12.6 Hz, 2H), 3.11 (t, J = 11.9 Hz, 2H), 2.82-2.75 (m, |
| | | 1H), 2.51 (p, J = 1.9 Hz, 3H), |
| | | 2.14 (dd, J = 6.7, 3.2 Hz, 1H), |
| | | 2.02 (d, J = 12.4 Hz, 2H), 1.50 (t, |
| | | J = 11.4 Hz, 2H), 0.41 (td, J = |
| | | 6.4, 4.1 Hz, 2H), 0.27-0.21 (m, |
| | | 2H). |
| 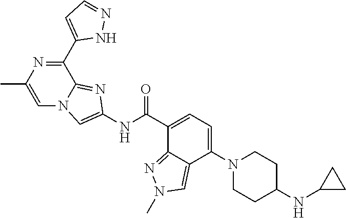 [SMILES: Cc1cn2cc(NC(=O)c3ccc(N4CCC(NC5CC5)CC4)c4cn(C)nc34)nc2c(-c2ccn[nH]2)n1] Compound 487 | 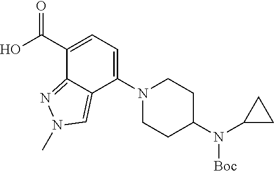 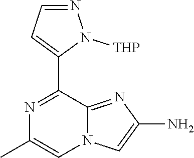 D53 | Modification: Step 1 was ran at room temperature for 16 h. Step 2 was ran for 1 h at room temperature. The residue was purified by reversed-phase flash chromatography (Condition 4, Gradient 7) to afford 4-(4- (cyclopropyl-amino)piperidin-1- yl)-2-methyl-N-(6-methyl-8- (1H-pyrazol-5-yl)imidazo[1,2- a]-pyrazin-2-yl)-2H-indazole-7- carboxamide 2,2,2- trifluoroacetate (Compound 487, 23 mg, 36.8 μmol). LCMS (ES, m/z): 511 [M + H]⁺. ¹H NMR (400 MHz, DMSO-d₆) δ 11.62 (s, 1H), 8.89 (s, 1H), 8.78 (s, 2H), 8.49 (d, J = 10.2 Hz, 2H), 8.08 (d, J = 8.1 Hz, 1H), 7.80 (d, J = 2.0 Hz, 1H), 7.53 (d, J = 1.9 Hz, 1H), 6.59 (d, J = 8.2 Hz, 1H), 4.35 (s, 3H), 4.08 (d, J = 12.9 Hz, 2H), 3.50 (s, 1H), 3.08 |
| | | (t, J = 12.3 Hz, 2H), 2.80 (s, 1H), |
| | | 2.50 (d, J = 1.9 Hz, 3H), 2.21 (d, |
| | | J = 12.1 Hz, 2H), 1.91-1.55 (m, |
| | | 2H), 0.85 (dd, J = 7.5, 2.8 Hz, |
| | | 4H). |
| 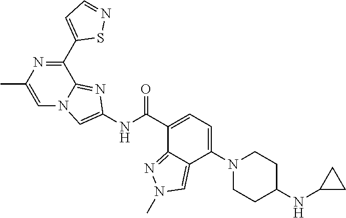 [SMILES: Cc1cn2cc(NC(=O)c3ccc(N4CCC(NC5CC5)CC4)c4cn(C)nc34)nc2c(-c2ccns2)n1] Compound 492 | 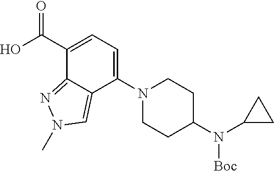 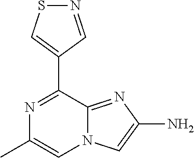 D54 | Modification: Step 1 was ran at room temperature for 6 h. Step 2 was ran using TMSOTf and DIEA in DCM for 1 h at room temperature. The residue was purified by reversed-phase flash chromatography (Condition 3, Gradient 20) to afford 4-[4- (cyclopropylamino)-1- piperidyl]-N-(8-isothiazol-4-yl- 6-methyl-imidazo[1,2-a]- pyrazin-2-yl)-2-methyl- indazole-7-carboxamide (Compound 492, 18 mg, 34.1 μmol). LCMS (ES, m/z): 528 [M + H]⁺. ¹H NMR (300 MHz, DMSO-d₆) δ 11.65 (s, 1H), 10.20 (s, 1H), 9.55 (s, 1H), 8.81 (s, 1H), 8.54-8.44 (m, 2H), 8.04 (d, J = 8.1 Hz, 1H), 6.51 (d, J = 8.3 Hz, 1H), 4.34 (s, 3H), 3.91 (d, J = |
| | | 12.8 Hz, 2H), 3.09 (t, J = 11.5 Hz, |
| | | 2H), 2.94-2.72 (m, 1H), 2.51 |
| | | (d, J = 5.5 Hz, 3H), 2.14 (tt, J = |
| | | 6.7, 3.6 Hz, 1H), 2.02 (d, J = |
| | | 12.6 Hz, 2H), 1.49 (q, J = |
| | | 10.7 Hz, 2H), 0.41 (dt, J = 6.1, |
| | | 2.9 Hz, 2H), 0.30-0.19 (m, 2H). |

### Example 34: Synthesis of Compound 214

### Synthesis of Intermediate A179

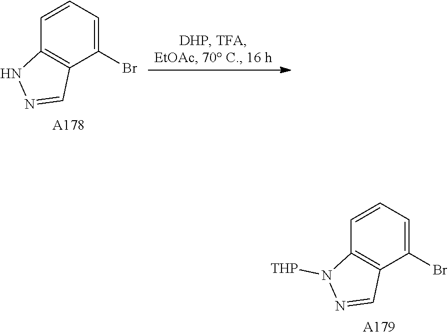

To a solution of 4-bromo-1H-indazole (A178, 2 g, 10.15 mmol) in EtOAc (20 mL) were added TFA (116 mg, 1.02 mmol) and DHP (1.83 g, 50.75 mmol), and the reaction was stirred for 16 h at 70° C. under N₂. The resulting mixture was concentrated under reduced pressure and purified by silica gel column chromatography (50% EtOAc in PE) to afford 4-bromo-1-tetrahydropyran-2-yl-indazole (A179, 2.5 g, 8.9 mmol). LCMS (ES, m/z): 281 [M+H]⁺.

### Synthesis of Intermediate A180

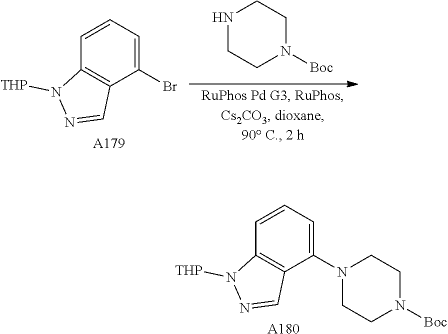

To a solution of Intermediate A179 (1 g, 3.56 mmol) and tert-butyl piperazine-1-carboxylate (994 mg, 5.3 mmol) in dioxane (10 mL) were added Ruphos (332 mg, 711.4 μmol), RuPhos Pd G3 (297 mg, 355.7 μmol) and Cs₂CO₃ (3.48 g, 10.7 mmol), and the reaction was stirred for 2 h at 90° C. under N₂. The resulting mixture was concentrated under reduced pressure and purified by silica gel column chromatography (50% EtOAc in PE) to afford tert-butyl 4-(1-tetrahydropyran-2-ylindazol-4-yl)piperazine-1-carboxylate (A180, 1 g, 2.59 mmol). LCMS (ES, m/z): 387 [M+H]⁺.

### Synthesis of Intermediate A181

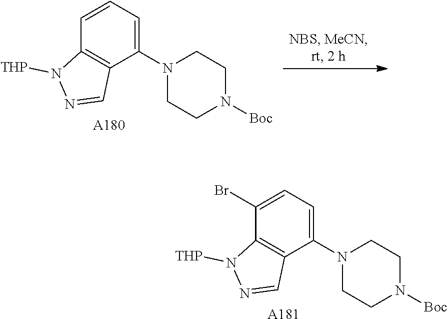

To a stirred solution of Intermediate A180 (1 g, 2.6 mmol) in MeCN (10 mL) was added NBS (368 mg, 2.1 mmol) in portions at 0° C., and the reaction was stirred for 2 h at room temperature. The resulting mixture was quenched with water (50 mL) and extracted with EtOAc (3×20 mL). The combined organics were washed with brine (10 mL), dried over anhydrous Na₂SO₄, and filtered. The filtrate was concentrated under reduced pressure and purified by silica gel column chromatography (33% EtOAc in PE) to afford tert-butyl 4-(7-bromo-1-tetrahydropyran-2-yl-indazol-4-yl)piperazine-1-carboxylate (A181, 600 mg, 1.29 mmol). LCMS (ES, m/z): 465 [M+H]⁺.

### Synthesis of Intermediate A182

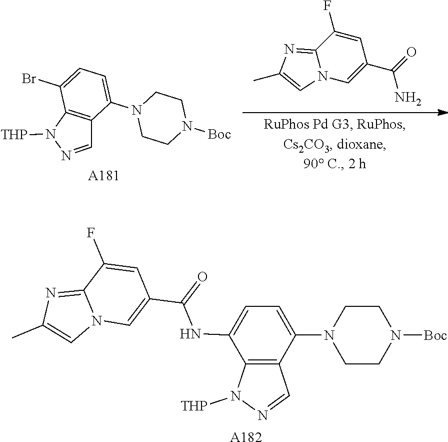

To a solution of Intermediate A181 (200 mg, 429.8 μmol) and 8-fluoro-2-methylimidazo[1,2-a]pyridine-6-carboxamide (125 mg, 644.6 μmol) in dioxane (2 mL) were added Ruphos (40 mg, 85.95 μmol), RuPhos Pd G3 (36 mg, 42.98 μmol) and Cs₂CO₃ (420 mg, 1.29 mmol), and the reaction was stirred for 2 h at 90° C. under N₂. The resulting mixture was concentrated under reduced pressure and purified by silica gel column chromatography (67% EtOAc in PE) to afford tert-butyl 4-[7-[(8-fluoro-2-methyl-imidazo[1,2-a]pyridine-6-carbonyl)amino]-1-tetrahydropyran-2-yl-indazol-4-yl]piperazine-1-carboxylate (A182, 150 mg, 259.7 μmol, 60% yield). LCMS (ES, m/z): 578 [M+H]⁺.

### Synthesis of Compound 214

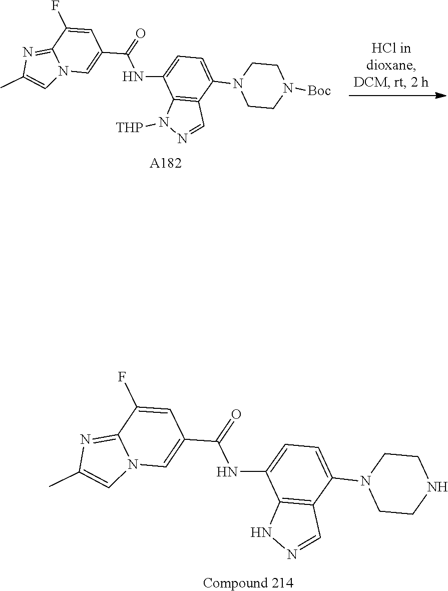

To a stirred solution of Intermediate A182 (150 mg, 259.7 μmol) in DCM (2 mL) was added HCl (4.0 M in 1,4-dioxane, 0.5 mL) dropwise at 0° C., and the reaction was stirred for 2 h at room temperature. The resulting mixture was concentrated under reduced pressure and purified by Prep-HPLC (Condition 1, Gradient 1) to afford 8-fluoro-2-methyl-N-(4-piperazin-1-yl-1H-indazol-7-yl) imidazo[1,2-a]pyridine-6-carboxamide (Compound 214, 57.8 mg, 146.9 μmol). LCMS (ES, m/z): 394 [M+H]⁺. ¹H NMR (400 MHz, DMSO-d₆) δ 12.86 (s, 1H), 10.21 (s, 1H), 9.13 (d, J=1.4 Hz, 1H), 8.11 (s, 1H), 8.01 (d, J=2.8 Hz, 1H), 7.70 (dd, J=12.1, 1.4 Hz, 1H), 7.28 (d, J=7.9 Hz, 1H), 6.44 (d, J=8.0 Hz, 1H), 3.17 (t, J=4.8 Hz, 4H), 2.96 (t, J=4.8 Hz, 4H), 2.41 (d, J=0.9 Hz, 3H).

### Example 35: Synthesis of Compound 215

### Synthesis of Intermediate A183

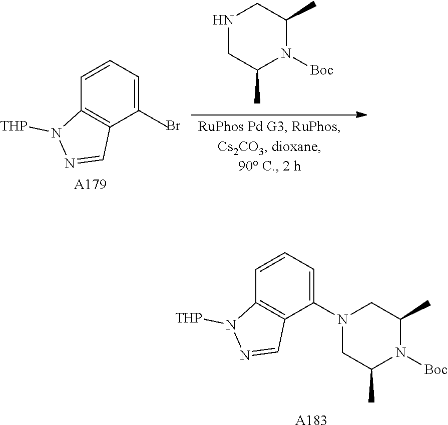

To a solution of Intermediate A179 (1 g, 3.56 mmol) and tert-butyl(2S,6R)-2,6-dimethylpiperazine-1-carboxylate (1.14 g, 5.34 mmol) in dioxane (10 mL) were added RuPhos Pd G3 (297 mg, 355.7 μmol), RuPhos (332 mg, 711.4 μmol) and Cs₂CO₃ (3.48 g, 10.7 mmol), and the reaction was stirred for 2 h at 90° C. under N₂. The resulting mixture was concentrated under reduced pressure and purified by silica gel column chromatography (25% EtOAc in PE) to afford tert-butyl (2R,6S)-2,6-dimethyl-4-(1-tetrahydropyran-2-ylindazol-4-yl)piperazine-1-carboxylate (A183, 900 mg, 2.17 mmol). LCMS (ES, m/z): 415 [M+H]⁺.

### Synthesis of Intermediate A184

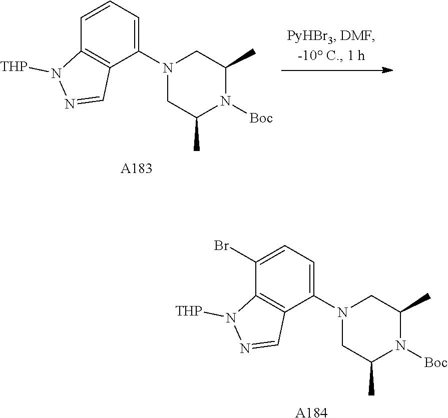

To a stirred solution of Intermediate A183 (900 mg, 2.17 mmol) in DMF (10 mL) was added PyHBr₃ (555 mg, 1.74 mmol) in portions at −10° C. The resulting mixture was stirred for 1 h at −10° C., quenched with saturated aqueous NaHCO₃ (20 mL), and subsequently extracted with EtOAc (3×30 mL). The combined organics were washed with brine (10 mL), dried over anhydrous Na₂SO₄, and filtered. The filtrate was concentrated under reduced pressure and purified by reverse flash chromatography (Condition 2, Gradient 1) to afford tert-butyl (2R,6S)-4-(7-bromo-1-tetrahydropyran-2-yl-indazol-4-yl)-2,6-dimethyl-piperazine-1-carboxylate (A184, 500 mg, 1.01 mmol). LCMS (ES, m/z): 493 [M+H]⁺.

### Synthesis of Intermediate A185

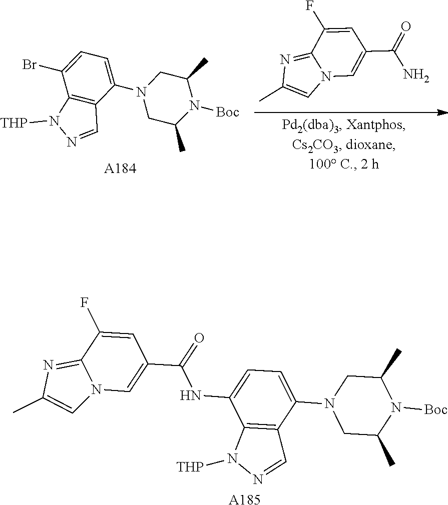

To a solution of Intermediate A184 (200 mg, 405.3 μmol) and 8-fluoro-2-methylimidazo[1,2-a]pyridine-6-carboxamide (117 mg, 608 μmol) in dioxane (2 mL) were added Pd₂(dba)₃ (37 mg, 40.53 μmol), XantPhos (47 mg, 81.06 μmol) and Cs₂CO₃ (396 mg, 1.22 mmol), and the reaction was stirred for 2 h at 100° C. under N₂. The resulting mixture was concentrated under reduced pressure and purified by silica gel column chromatography (50% EtOAc in PE) to afford tert-butyl(2S,6R)-4-[7-[(8-fluoro-2-methyl-imidazo[1,2-a]pyridine-6-carbonyl)amino]-1-tetrahydropyran-2-yl-indazol-4-yl]-2,6-dimethyl-piperazine-1-carboxylate (A185, 140 mg, 231.1 μmol). LCMS (ES, m/z): 606 [M+H]⁺.

### Synthesis of Compound 215

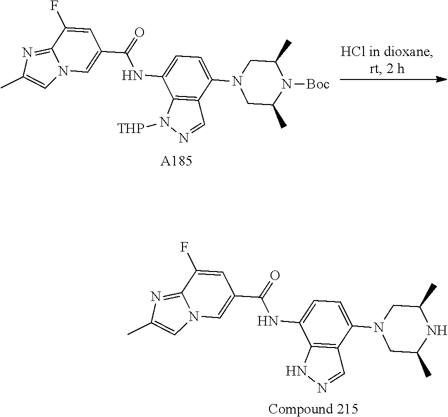

To a stirred solution of Intermediate A185 (140 mg, 231.1 μmol) in DCM (2 mL) was added 4M HCl in dioxane (0.5 mL) at 0° C., and the reaction was stirred for 2 h at room temperature. The resulting mixture was concentrated under reduced pressure and purified by Prep-HPLC (Condition 1, Gradient 1) to afford N-[4-[(3R,5S)-3,5-dimethylpiperazin-1-yl]-1H-indazol-7-yl]-8-fluoro-2-methyl-imidazo[1,2-a]pyridine-6-carboxamide (Compound 215, 41.8 mg, 99.2 μmol). LCMS (ES, m/z): 422 [M+H]⁺. ¹H NMR (400 MHz, DMSO-d₆) δ 12.85 (s, 1H), 10.20 (s, 1H), 9.13 (d, J=1.4 Hz, 1H), 8.12 (s, 1H), 8.01 (d, J=3.4 Hz, 1H), 7.70 (dd, J=12.1, 1.4 Hz, 1H), 7.27 (d, J=7.9 Hz, 1H), 6.43 (d, J=8.0 Hz, 1H), 3.60-3.52 (m, 2H), 3.03 (dd, J=9.4, 6.7 Hz, 2H), 2.43-2.38 (m, 3H), 2.30 (t, J=10.8 Hz, 2H), 1.05 (d, J=6.3 Hz, 6H).

### Example 36: Synthesis of A187

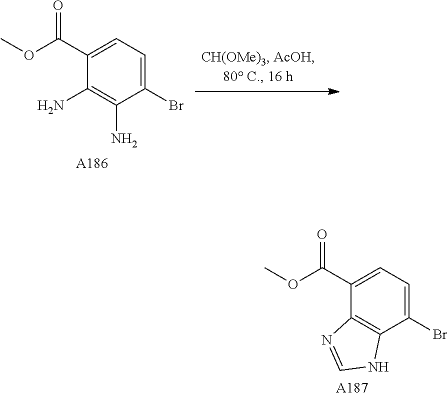

A solution of methyl 2,3-diamino-4-bromobenzoate (A186, 200 mg, 0.82 mmol, 1 eq) and CH(OMe)₃ (2 mL) in AcOH (4 mL) was stirred for 16 h at 80° C. and subsequently quenched with water (10 mL). The resulting solids were collected by filtration and washed with ether (2×5 mL) to afford methyl 7-bromo-1H-1,3-benzodiazole-4-carboxylate (A187, 160 mg). LCMS (ES, m/z): 255 [M+H]⁺.

### Example 37: Synthesis of Compounds 220, 221

### Synthesis of Intermediate A188

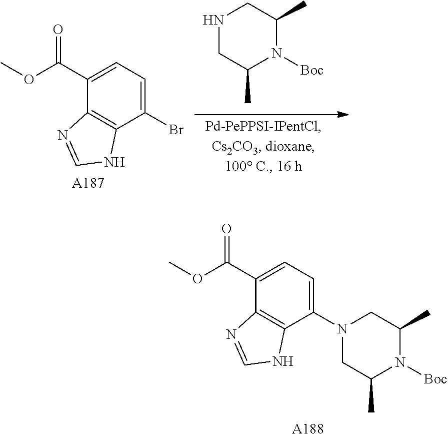

To a stirred solution of Intermediate A187 (160 mg, 0.63 mmol, 1 eq) and tert-butyl (2R,6S)-2,6-dimethylpiperazine-1-carboxylate (202 mg, 0.94 mmol, 1.5 eq) in dioxane (5 mL) were added Cs₂CO₃ (409 mg, 1.25 mmol, 2 eq) and Pd-PEPPSI-IPentCl 2-methylpyridine (o-picoline) (53 mg, 0.06 mmol, 0.1 eq), and the reaction was stirred for 16 h at 100° C. under N₂. The resulting mixture was allowed to cool to room temperature, concentrated under reduced pressure, and purified by silica gel column chromatography (50% EtOAc in PE) to afford methyl 7-[(3R,5S)-4-(tert-butoxycarbonyl)-3,5-dimethylpiperazin-1-yl]-1H-1,3-benzodiazole-4-carboxylate (A188, 155 mg). LCMS (ES, m/z): 389 [M+H]⁺.

### Synthesis of Intermediate A189

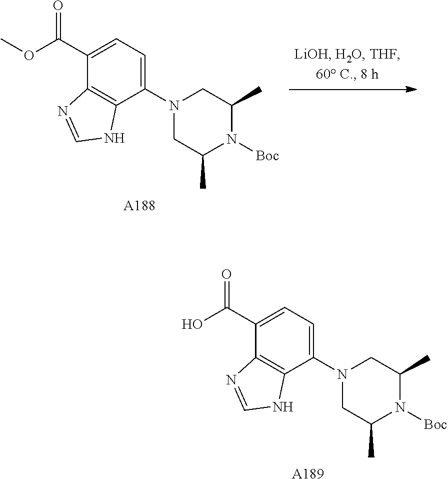 [SMILES: COC(=O)c1ccc(N2C[C@@H](C)N(C(=O)OC(C)(C)C)[C@@H](C)C2)c2[nH]cnc12 ; "A188" ; C[C@@H]1CN(c2ccc(C(=O)O)c3nc[nH]c23)C[C@H](C)N1C(=O)OC(C)(C)C ; "A189"]

To a stirred solution of Intermediate A188 (155 mg, 0.4 mmol, 1 eq) in THF (3 mL) and H₂O (1.5 mL) was added LiOH·H₂O (168 mg, 4 mmol, 10 eq) at room temperature. The resulting mixture was stirred for 8 h at 60° C. and acidified to pH 7 with 1N HCl (aq.). The resulting solids were collected by filtration and washed with water (2×10 mL) to afford 7-[(3R,5S)-4-(tert-butoxycarbonyl)-3,5-dimethylpiperazin-1-yl]-1H-1,3-benzodiazole-4-carboxylic acid (A189, 120 mg). LCMS (ES, m/z): 375 [M+H]⁺.

### Synthesis of Intermediate A190

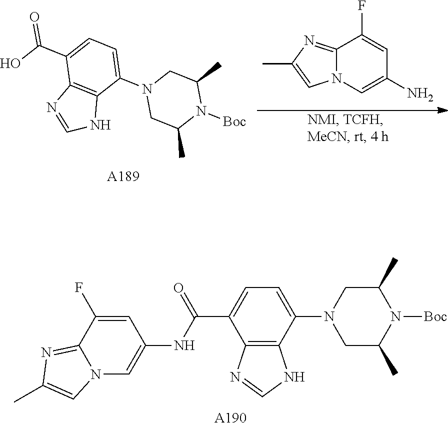

To a stirred solution of Intermediate A189 (120 mg, 0.32 mmol, 1 eq) and NMI (132 mg, 1.6 mmol, 5 eq) in MeCN (4 mL) were added TCFH (108 mg, 0.38 mmol, 1.2 eq) and 8-fluoro-2-methylimidazo[1,2-a]pyridin-6-amine dihydrochloride (69 mg, 0.42 mmol, 1.3 eq) at room temperature. The resulting mixture was stirred for 4 h, and the reaction was quenched by the addition of water (20 mL). The precipitated solids were collected by filtration and washed with water (3×5 mL) to afford tert-butyl (2R,6S)-4-[7-((8-fluoro-2-methylimidazo[1,2-a]pyridin-6-yl)carbamoyl)-3H-1,3-benzodiazol-4-yl]-2,6-dimethylpiperazine-1-carboxylate (A190, 115 mg). LCMS (ES, m/z): 522 [M+H]⁺.

### Synthesis of Compound 220

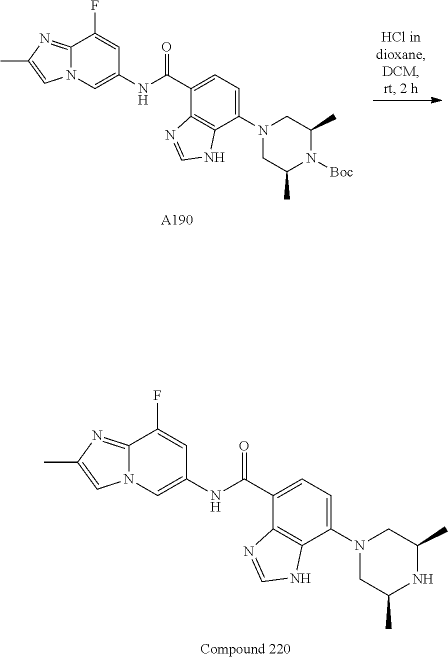

A solution of Intermediate A190 (115 mg, 0.22 mmol, 1 eq) and 4 M HCl (gas) in 1,4-dioxane (1 mL) in DCM (5 mL) was stirred for 2 h at room temperature. The resulting mixture was concentrated under reduced pressure and purified by reversed-phase flash chromatography (Condition, Gradient) to afford 7-[(3R,5S)-3,5-dimethylpiperazin-1-yl]-N-(8-fluoro-2-methyl-imidazo[1,2-a]pyridin-6-yl)-1H-1,3-benzodiazole-4-carboxamide (Compound 220, 57.3 mg). LCMS (ES, m/z): 422 [M+H]⁺. ¹H NMR (300 MHz, Methanol-d₄) δ 9.08 (s, 1H), 8.22 (s, 1H), 7.93 (d, J=8.0 Hz, 1H), 7.70 (s, 1H), 7.31 (dd, J=12.1, 5.6 Hz, 1H), 6.82 (s, 1H), 3.95 (s, 2H), 3.19 (s, 2H), 2.50 (t, J=11.1 Hz, 2H), 2.43 (t, J=1.3 Hz, 3H), 1.21 (dd, J=6.4, 1.2 Hz, 6H).

An analogous method was followed to obtain the following compounds.

| | Starting | |
|---|---|---|
| Compound | Material | Characterization |
| 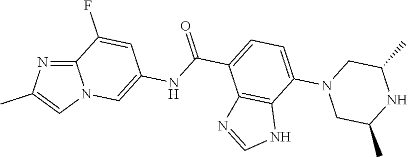 Compound 221 | 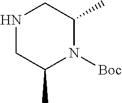 | Modifications: Step 2 was ran for 20 h. Step 3 was ran in DMF for δ h. The crude product was purified by reversed-phase flash chromatography (Condition 1, Gradient 3) to afford 7- [(3S,5S)-3,5-dimethylpiperazin-1-yl]-N- (8-fluoro-2-methylimidazo[1,2- a]pyridin-6-yl)-1H-1,3-benzodiazole-4- carboxamide (Compound 221, 47 mg). LCMS (ES, m/z): 422 [M + H]⁺. ¹H NMR (300 MHz, Methanol-d₄) δ 9.10 (s, 1H), 8.21 (s, 1H), 7.94 (d, J = 8.4 Hz, 1H), 7.73 |
| | | (s, 1H), 7.38-7.28 (m, 1H), 6.82 (s, 1H), |
| | | 3.69 (s, 3H), 3.50 (s, 3H), 2.44 (s, 3H), |
| | | 1.30 (d, J = 6.6 Hz, 6H). |

### Example 38: Synthesis of Compound 225

### Synthesis of Intermediate A192

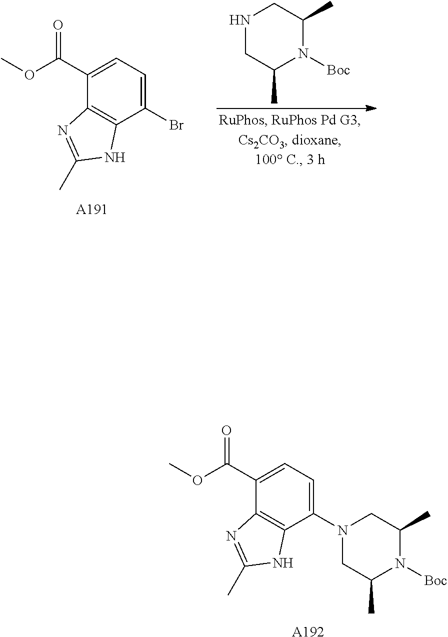

To a stirred solution of methyl 7-bromo-2-methyl-1H-1,3-benzodiazole-4-carboxylate (A191, 100 mg, 0.37 mmol, 1 eq) and tert-butyl (2R,6S)-2,6-dimethylpiperazine-1-carboxylate (120 mg, 0.56 mmol, 1.5 eq) in dioxane (3 mL) were added Cs₂CO₃ (243 mg, 0.74 mmol, 2 eq), RuPhos (35 mg, 0.07 mmol, 0.2 eq) and RuPhos Pd G3 (32 mg, 0.04 mmol, 0.1 eq), and the reaction was stirred for 3 h at 100° C. under N₂. The resulting mixture was concentrated under reduced pressure and purified by silica gel column chromatography (33% EtOAc in PE) to afford methyl 7-[(3R,5S)-4-(tert-butoxycarbonyl)-3,5-dimethylpiperazin-1-yl]-2-methyl-1H-1,3-benzodiazole-4-carboxylate (A192, 107 mg). LCMS (ES, m/z): 403 [M+H]⁺.

### Synthesis of Intermediate A193

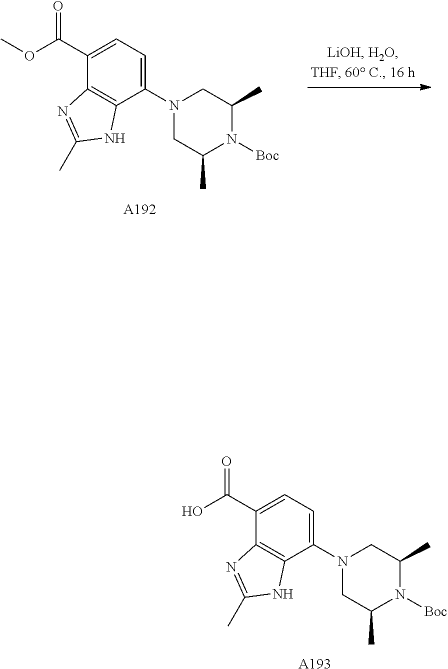 [SMILES: COC(=O)c1ccc(N2C[C@@H](C)N(C(=O)OC(C)(C)C)[C@@H](C)C2)c2[nH]c(C)nc12 ; "A192" ; Cc1nc2c(C(=O)O)ccc(N3C[C@@H](C)N(C(=O)OC(C)(C)C)[C@@H](C)C3)c2[nH]1 ; "A193"]

A solution of Intermediate A192 (107 mg, 0.27 mmol, 1 eq) and LiOH·H₂O (64 mg, 2.66 mmol, 10 eq), H₂O (1.5 mL) in THF (3 mL) was stirred for 16 h at 60° C. The mixture was acidified to pH 3 with 1N HCl (aq.) and extracted with EtOAc (3×10 mL). The combined organic layers were washed with brine (5 mL), dried over anhydrous Na₂SO₄, filtered, and concentrated under reduced pressure to afford 7-[(3R,5S)-4-(tert-butoxycarbonyl)-3,5-dimethylpiperazin-1-yl]-2-methyl-1H-1,3-benzodiazole-4-carboxylic acid (A193, 95 mg). LCMS (ES, m/z): 389 [M+H]⁺.

### Synthesis of Intermediate A194

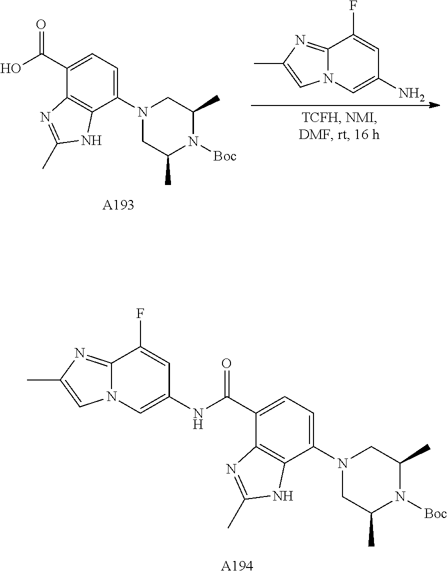

To a stirred solution of Intermediate A193 (95 mg, 0.25 mmol, 1 eq) and NMI (100 mg, 1.23 mmol, 5 eq) in DMF (3 mL) were added TCFH (82 mg, 0.29 mmol, 1.2 eq) and 8-fluoro-2-methylimidazo[1,2-a]pyridin-6-amine dihydrochloride (49 mg, 0.29 mmol, 1.2 eq), and the reaction was stirred for 16 h at room temperature. The resulting mixture was quenched by the addition of water (5 mL), and the precipitated solids were collected by filtration and washed with water (2×3 mL) to afford tert-butyl (2R,6S)-4-[7-((8-fluoro-2-methylimidazo[1,2-a]pyridin-6-yl)carbamoyl)-2-methyl-3H-1,3-benzodiazol-4-yl]-2,6-dimethylpiperazine-1-carboxylate (A194, 80 mg). LCMS (ES, m/z): 536 [M+H]⁺.

### Synthesis of Compound 225

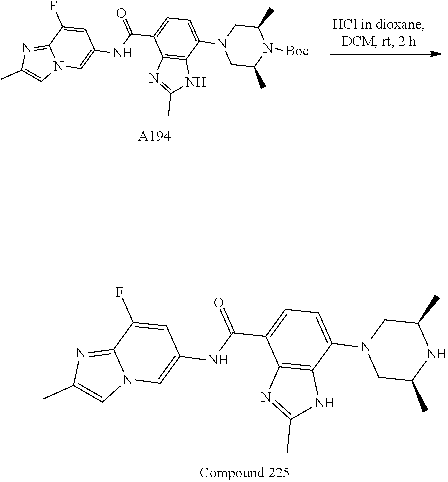

A solution of Intermediate A194 (80 mg, 0.15 mmol, 1 eq) and 4 M HCl in 1,4-dioxane (1 mL) in DCM (4 mL) was stirred for 2 h at room temperature. The resulting mixture was concentrated under reduced pressure and purified by reversed-phase flash chromatography (Condition 1, Gradient 5) to afford 7-[(3R,5S)-3,5-dimethylpiperazin-1-yl]-N-(8-fluoro-2-methylimidazo[1,2-a]pyridin-6-yl)-2-methyl-1H-1,3-benzodiazole-4-carboxamide (Compound 225, 17.4 mg). LCMS (ES, m/z): 436 [M+H]⁺. ¹H NMR (300 MHz, Methanol-d₄) δ 9.10 (s, 1H), 7.88 (s, 1H), 7.72 (s, 1H), 7.31 (d, J=11.5 Hz, 1H), 6.81 (s, 1H), 3.22 (s, 4H), 2.70 (s, 3H), 2.46 (d, J=11.9 Hz, 5H), 1.25-1.17 (m, 6H).

An analogous method was followed to obtain the following compounds.

| | Starting | |
|---|---|---|
| Compound | Material | Characterization |
| 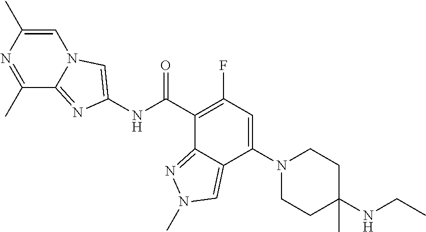 [SMILES: CCNC1(C)CCN(c2cc(F)c(C(=O)Nc3cn4cc(C)nc(C)c4n3)c3nn(C)cc23)CC1] Compound 375 | 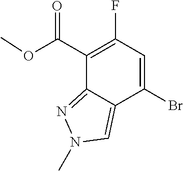 A99 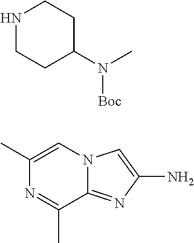 | Modification: Step 1 was ran for 16 h at 90°C. Step 2 was ran at room temperature. Step 3 was ran in MeCN at room temperature for 16 h. The crude product was purified by Prep- HPLC (Condition 5, Gradient 13) to afford N-(6,8-dimethylimidazo[1,2-a]pyrazin-2- yl)-4-[4-(ethylamino)-4-methyl-1- piperidyl]-6-fluoro-2-methyl-indazole-7- carboxamide (Compound 375, 9.6 mg, 20.1 umol, 15% yield) as a solid. LCMS (ES, m/z): 479 [M + H]⁺. ¹H NMR (400 MHz, Methanol-d₄) δ 8.55 (s, 1H), 8.30 (s, 1H), 8.19 (s, 1H), 6.25 (d, J = 16.2 Hz, 1H), 4.33 (s, 3H), 3.81 (dt, J = 13.3, 4.9 Hz, 2H), 3.42 (ddd, J = 13.0, 9.3, 3.6 Hz, 2H), 2.80 (s, 3H), 2.72 (q, J = 7.1 Hz, 2H), 2.46 (d, J = 1.1 Hz, 3H), 1.93-1.76 (m, 4H), 1.29 (s, 3H), 1.19 (t, J = 7.2 Hz, 3H). |

### Example 39: Synthesis of Compound 230

### Synthesis of Intermediate A196

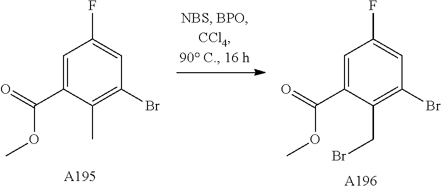

To a stirred solution of methyl 3-bromo-5-fluoro-2-methylbenzoate (A195, 1 g, 4.05 mmol, 1 eq) and BPO (0.10 g, 0.41 mmol, 0.1 eq) in CCl₄ (10 mL) was added NBS (0.74 g, 4.17 mmol, 1.03 eq) at room temperature under N₂. The resulting mixture was stirred for 16 h at 90° C. under N2, allowed to cool to room temperature, and subsequently diluted with DCM (50 mL). The resulting mixture was washed with 2 saturated NaHCO₃ (aq.) (2×30 mL), dried over anhydrous Na₂SO₄, filtered, and concentrated under reduced pressure to afford methyl 3-bromo-2-(bromomethyl)-5-fluorobenzoate (A196, 1.29 g). LCMS (ES, m/z): 325 [M+H]⁺.

### Synthesis of Intermediate A197

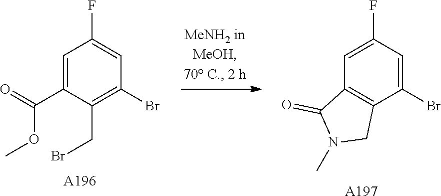

A solution of methyl 3-bromo-2-(bromomethyl)-5-fluorobenzoate (A196, 1.29 g, 3.96 mmol, 1 eq) and 2M methylamine solution in MeOH (10 mL) was stirred for 2 h at 70° C. The resulting mixture was allowed to cool to room temperature, concentrated under reduced pressure, and purified by silica gel column chromatography (50% EtOAc in PE) to afford 4-bromo-6-fluoro-2-methyl-3H-isoindol-1-one (A197, 0.9 g). LCMS (ES, m/z): 325 [M+H]⁺.

### Synthesis of Intermediate A198

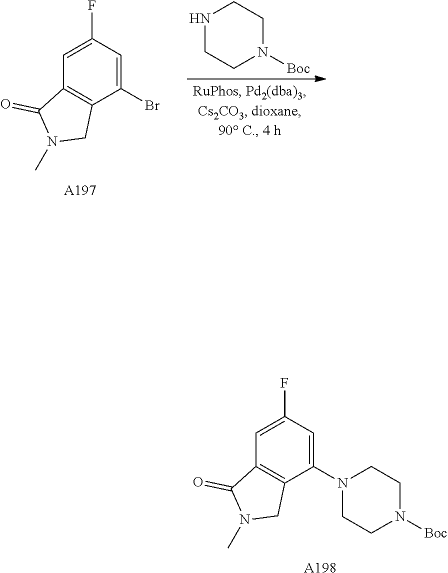

To a stirred solution of Intermediate A197 (0.9 g, 4.10 mmol), Cs₂CO₃ (2.67 g, 8.19 mmol) and tert-butyl piperazine-1-carboxylate (915 mg, 4.92 mmol) in dioxane (20 mL) were added Ruphos (382 mg, 819.5 μmol) and Pd₂(dba)₃ (376 mg, 409.7 μmol) at room temperature under N₂, stirred for 4 h at 90° C. under N₂. The resulting mixture was concentrated under reduced pressure and purified by reverse flash chromatography (Condition 2, Gradient) to afford tert-butyl 4-(6-fluoro-2-methyl-1-oxo-isoindolin-4-yl)piperazine-1-carboxylate (A198, 0.8 g, 2.29 mmol). LCMS (ES, m/z): 350 [M+H]⁺.

### Synthesis of Intermediate A199

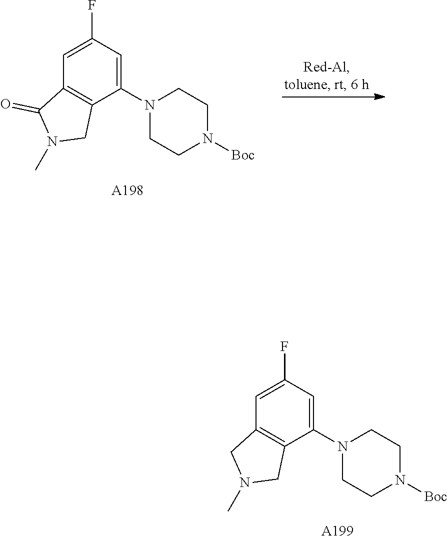 [SMILES: CN1Cc2c(cc(F)cc2N2CCN(C(=O)OC(C)(C)C)CC2)C1=O ; "A198" ; CN1Cc2cc(F)cc(N3CCN(C(=O)OC(C)(C)C)CC3)c2C1 ; "A199"]

To a stirred solution of Intermediate A198 (0.8 g, 2.29 mmol) in toluene (20 mL) was added Red-A1 (1.98 g, 6.87 mmol, 70%) dropwise at 0° C. The resulting mixture was stirred for 16 h at room temperature, quenched by the addition of ice water (40 mL), and then extracted with EtOAc (3×10 mL). The combined organic layers were washed with brine (5 mL), dried over anhydrous Na₂SO₄, and filtered. The filtrate was concentrated under reduced pressure and purified by silica gel column chromatography (67% EtOAc in PE) to afford tert-butyl 4-(6-fluoro-2-methyl-isoindolin-4-yl)piperazine-1-carboxylate (A199, 0.35 g, 1.04 mmol). LCMS (ES, m/z): 336 [M+H]⁺.

### Synthesis of Intermediate A200

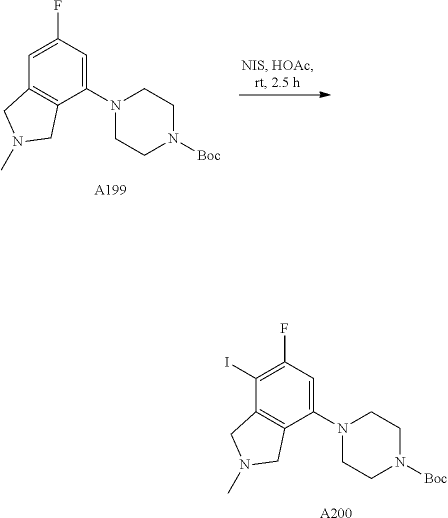 [SMILES: CN1Cc2cc(F)cc(N3CCN(C(=O)OC(C)(C)C)CC3)c2C1 ; "A199" ; CN1Cc2c(N3CCN(C(=O)OC(C)(C)C)CC3)cc(F)c(I)c2C1 ; "A200"]

A solution of Intermediate A199 (0.32 g, 954.0 μmol) and NIS (257 mg, 1.14 mmol) in CH₃COOH (6 mL) was stirred for 3 h at room temperature, later quenched by the addition of water (20 mL), and then extracted with EtOAc (3×10 mL). The combined organic layers were washed with brine (5 mL), dried over anhydrous Na₂SO₄, and filtered. The filtrate was concentrated under reduced pressure and purified by silica gel column chromatography (5% MeOH in DCM) to afford tert-butyl 4-(6-fluoro-7-iodo-2-methyl-isoindolin-4-yl)piperazine-1-carboxylate (A200, 0.32 g, 693.7 μmol). LCMS (ES, m/z): 462 [M+H]⁺.

### Synthesis of Intermediate A201

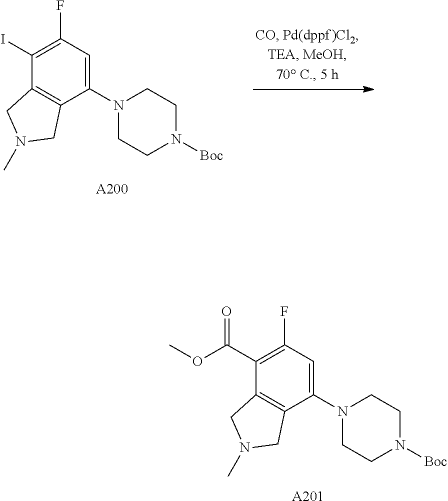 [SMILES: CN1Cc2c(N3CCN(C(=O)OC(C)(C)C)CC3)cc(F)c(I)c2C1 ; "A200" ; COC(=O)c1c(F)cc(N2CCN(C(=O)OC(C)(C)C)CC2)c2c1CN(C)C2 ; "A201"]

To a solution of Intermediate A200 (0.32 g, 693.7 μmol) in MeOH (10 mL) was added TEA (351 mg, 3.47 mmol) and Pd(dppf)Cl₂ (57 mg, 69.37 μmol) in a pressure tank. The mixture was purged with nitrogen for 1 min and then was pressurized to 20 atm with carbon monoxide at 70° C. for 5 h. The reaction mixture was cooled to room temperature and filtered to remove insoluble solids. The filtrate was concentrated under reduced pressure and purified by silica gel column chromatography (67% EtOAc in DCM) to afford methyl 7-(4-tert-butoxycarbonylpiperazin-1-yl)-5-fluoro-2-methyl-isoindoline-4-carboxylate (A201, 0.18 g, 457.5 μmol). LCMS (ES, m/z): 394 [M+H]⁺.

### Synthesis of Intermediate A202

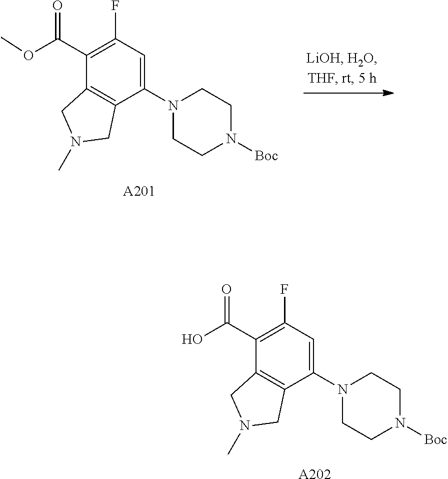 [SMILES: COC(=O)c1c(F)cc(N2CCN(C(=O)OC(C)(C)C)CC2)c2c1CN(C)C2 ; "A201" ; CN1Cc2c(N3CCN(C(=O)OC(C)(C)C)CC3)cc(F)c(C(=O)O)c2C1 ; "A202"]

To a stirred solution of Intermediate A201 (0.18 g, 457.5 μmol) and H₂O (2 mL) in THF (4 mL) was added LiOH·H₂O (192 mg, 4.57 mmol), and the reaction was stirred for 5 h at room temperature. The resulting mixture was quenched by the addition of water (15 mL), acidified to pH 4 with 1M HCl (aq.), and then extracted with EtOAc (3×10 mL). The combined organic layers were washed with brine (5 mL), dried over anhydrous Na₂SO₄, filtered, and concentrated under reduced pressure to afford 7-(4-tert-butoxycarbonylpiperazin-1-yl)-5-fluoro-2-methyl-isoindoline-4-carboxylic acid (A202, 0.12 g, 316.3 μmol). LCMS (ES, m/z): 380 [M+H]⁺.

### Synthesis of Intermediate A203

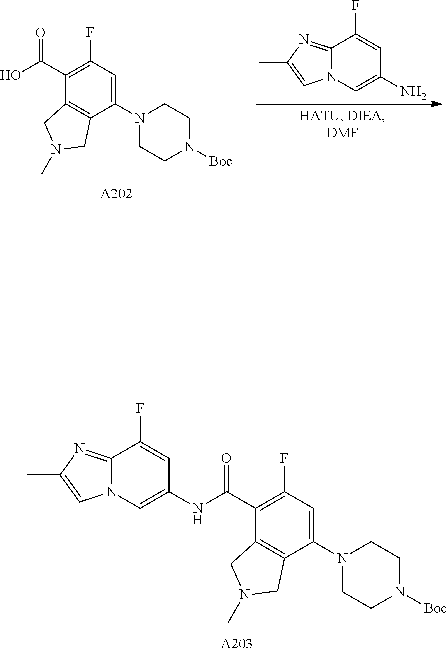

To a stirred solution of Intermediate A202 (0.12 g, 316.3 μmol) and 8-fluoro-2-methylimidazo[1,2-a]pyridin-6-amine dihydrochloride (75 mg, 316.3 μmol) in MeCN (4 mL) were added NMI (129 mg, 1.58 mmol, 125.4 L) and TCFH (106 mg, 379.5 μmol) at room temperature and stirred for 8 h before quenching with water (15 mL), followed by extraction with EtOAc (3×10 mL). The combined organic layers were washed with brine (5 mL), dried over anhydrous Na₂SO₄, and filtered. The filtrate was concentrated under reduced pressure and purified by silica gel column chromatography (50% THF in DCM) to afford tert-butyl 4-[6-fluoro-7-[(8-fluoro-2-methyl-imidazo[1,2-a]pyridin-6-yl)carbamoyl]-2-methyl-isoindolin-4-yl]-piperazine-1-carboxylate (A203, 85 mg, 161.4 μmol). LCMS (ES, m/z): 527 [M+H]⁺.

### Synthesis of Compound 230

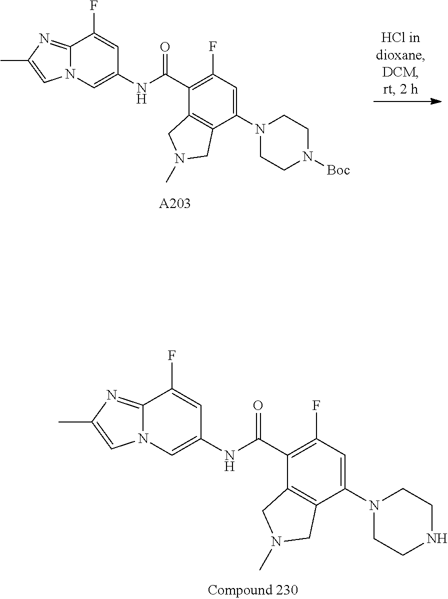

A solution of Intermediate A203 (85 mg, 161.4 μmol) and 4 M HCl (gas) in 1,4-dioxane (0.5 mL) in DCM (2.5 mL) was stirred for 2 h at room temperature. The resulting mixture was concentrated under reduced pressure and purified by reversed-phase flash chromatography (Condition 1, Gradient 5) to afford 5-fluoro-N-(8-fluoro-2-methyl-imidazo[1,2-a]pyridin-6-yl)-2-methyl-7-piperazin-1-yl-isoindoline-4-carboxamide (Compound 230, 10 mg, 23.5 μmol). LCMS (ES, m/z): 427 [M+H]⁺. ¹H NMR (300 MHz, CDCl₃) δ 9.01 (s, 1H), 8.18 (d, J=16.7 Hz, 1H), 7.41 (d, J=3.0 Hz, 1H), 6.74 (dd, J=11.0, 1.7 Hz, 1H), 6.53 (d, J=15.8 Hz, 1H), 4.32 (s, 2H), 3.86 (s, 2H), 3.07 (q, J=6.0 Hz, 8H), 2.64 (s, 3H), 2.49 (s, 3H).

### Example 40: Synthesis of Compound 231

### Synthesis of Intermediate A205

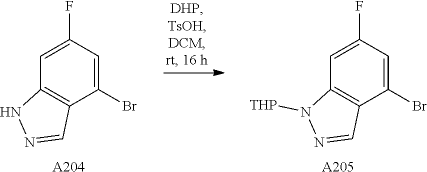

To a solution of 4-bromo-6-fluoro-1H-indazole (A204, 1 g, 4.65 mmol) and 3,4-dihydro-2H-pyran (548 mg, 6.51 mmol, 591.5 L) in DCM (10 mL) were added TsOH (80 mg, 465.1 μmol), and the reaction was stirred for 16 h at room temperature. The resulting mixture was concentrated under reduced pressure and purified by silica gel column chromatography (25% EtOAc in PE) to afford 4-bromo-6-fluoro-1-tetrahydropyran-2-yl-indazole (A205, 1.3 g, 4.35 mmol). LCMS (ES, m/z): 299 [M+H]⁺.

### Synthesis of Intermediate A206

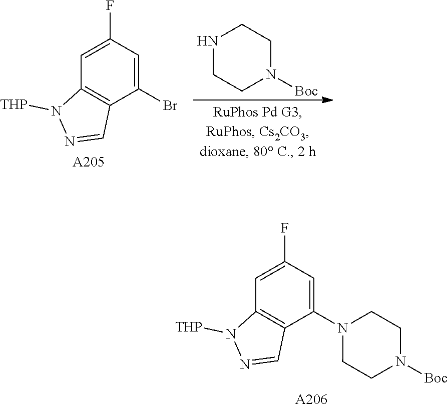

To a solution of Intermediate A205 (1 g, 3.34 mmol, 1 eq) and tert-butyl piperazine-1-carboxylate (0.62 g, 3.34 mmol, 1 eq) in dioxane (10 mL) were added Cs₂CO₃ (4.36 g, 13.37 mmol, 4 eq), RuPhos (0.31 g, 0.67 mmol, 0.2 eq) and RuPhos Pd G3 (0.28 g, 0.33 mmol, 0.1 eq), and the reaction was stirred for 2 h at 80° C. under N₂. The resulting mixture was concentrated under reduced pressure and purified by silica gel column chromatography (50% EtOAc in PE) to afford tert-butyl 4-[6-fluoro-1-(oxan-2-yl)indazol-4-yl]piperazine-1-carboxylate (A206, 1.1 g). LCMS (ES, m/z): 405 [M+H]⁺.

### Synthesis of Intermediate A207

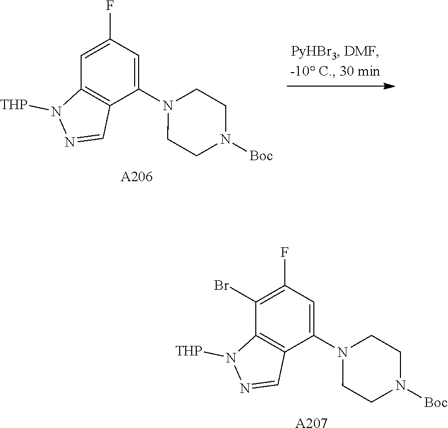

To a stirred solution of Intermediate A206 (0.9 g, 2.23 mmol) in DMF (10 mL) was added a solution of PyHBr₃ (640 mg, 2 mmol) in DMF (10 mL) dropwise at −10° C. The resulting mixture was stirred for 30 min at −10° C., diluted with water (150 mL), and subsequently extracted with EtOAc (3×30 mL). The combined organic layers were washed with water (2×10 mL), dried over anhydrous Na₂SO₄, and filtered. The filtrate was concentrated under reduced pressure and purified by silica gel column chromatography (33% EtOAc in PE) to afford tert-butyl 4-(7-bromo-6-fluoro-1-tetrahydropyran-2-yl-indazol-4-yl)piperazine-1-carboxylate (A207, 0.8 g, 1.66 mmol). LCMS (ES, m/z): 483 [M+H]⁺.

### Synthesis of Intermediate A208

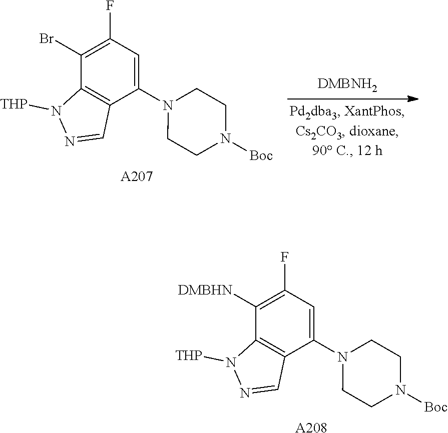

To a solution of Intermediate A207 (0.3 g, 620.6 μmol) and (2,4-dimethoxyphenyl)-methanamine (125 mg, 744.8 μmol) in dioxane (4 mL) were added Pd₂(dba)₃ (57 mg, 62.06 μmol), Xantphos (72 mg, 124.1 μmol) and Cs₂CO₃ (403 mg, 1240 μmol), and the reaction was stirred for 12 h at 90° C. under N₂. The resulting mixture was concentrated under reduced pressure and purified by reversed-phase flash chromatography (Condition 1, Gradient 6) to afford tert-butyl4-[7-[(2,4-dimethoxyphenyl)methylamino]-6-fluoro-1-tetrahydropyran-2-yl-indazol-4-yl]piperazine-1-carboxylate (A208, 0.24 g, 421.3 μmol). LCMS (ES, m/z): 570 [M+H]⁺.

### Synthesis of Intermediate A209

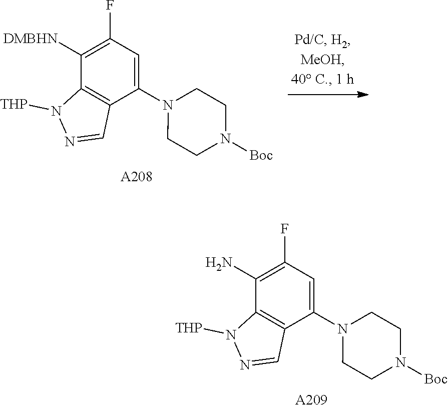

To a solution of Intermediate A208 (0.17 g, 316.2 μmol) in MeOH (6 mL) was added Pd/C 10% on Carbon (wetted with ca. 55% Water) (0.17 g) in a pressure tank. The resulting mixture was hydrogenated at 40° C. under 5 atm of hydrogen pressure for 1 h, then filtered through a Celite pad. The filtrate was concentrated under reduced pressure and purified by reversed-phase flash chromatography (Condition 1, Gradient 6) to afford tert-butyl 4-(7-amino-6-fluoro-1-tetrahydropyran-2-yl-indazol-4-yl)piperazine-1-carboxylate (A209, 97 mg, 231.2 μmol). LCMS (ES, m/z): 420 [M+H]⁺.

### Synthesis of Intermediate A210

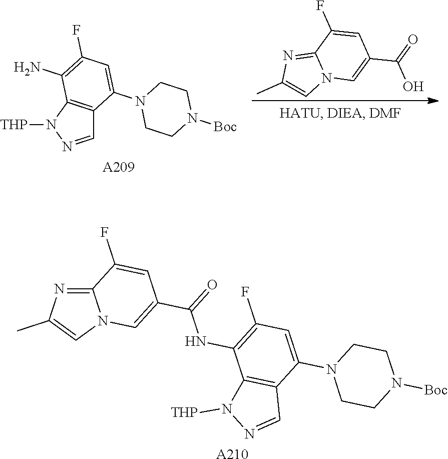

To a stirred solution of 8-fluoro-2-methyl-imidazo[1,2-a]pyridine-6-carboxylic acid (56 mg, 288.4 μmol) and Intermediate A209 (110 mg, 262.2 μmol) in DMF (5 mL) were added DIEA (101 mg, 786.7 μmol, 137.0 μL) and HATU (149 mg, 393.3 μmol) at room temperature. The resulting mixture was stirred for 3 h at room temperature and quenched with water (30 mL). The precipitated solids were collected by filtration and washed with water (3×10 mL) to afford tert-butyl 4-[6-fluoro-7-[(8-fluoro-2-methyl-imidazo[1,2-a]pyridine-6-carbonyl)amino]-1-tetrahydropyran-2-yl-indazol-4-yl]piperazine-1-carboxylate (A210, 0.09 g, 151.1 μmol) as a solid. LCMS (ES, m/z): 596 [M+H]⁺.

### Synthesis of Compound 231

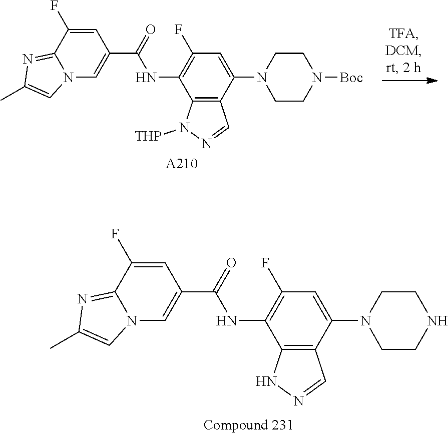

A solution of Intermediate A210 (90 mg, 151.1 μmol) and TFA (0.2 mL) in DCM (6 mL) was stirred for 1 h at room temperature. The resulting mixture was concentrated under reduced pressure and purified by reverse flash chromatography (Condition 1, Gradient) to afford 8-fluoro-N-(6-fluoro-4-piperazin-1-yl-1H-indazol-7-yl)-2-methyl-imidazo[1,2-a]pyridine-6-carboxamide (Compound 231, 21 mg, 51 μmol). LCMS (ES, m/z): 412 [M+H]⁺. ¹H NMR (300 MHz, DMSO-d₆) δ 13.08 (s, 1H), 10.10 (s, 1H), 9.11 (s, 1H), 8.14 (s, 1H), 8.01 (s, 1H), 7.69 (d, J=12.2 Hz, 1H), 6.36 (d, J=13.2 Hz, 1H), 3.22 (d, J=7.2 Hz, 4H), 2.93 (d, J=5.3 Hz, 4H), 2.41 (s, 3H).

### Example 41: Synthesis of Compound 263

### Synthesis of Intermediate A212

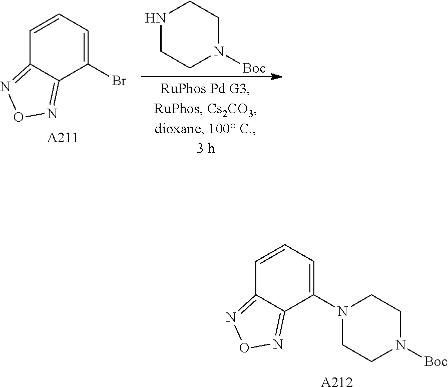

To a stirred solution of 4-bromo-2,1,3-benzoxadiazole (A211, 0.4 g, 2.01 mmol) and tert-butyl piperazine-1-carboxylate (449.2 mg, 2.41 mmol) in dioxane (5 mL) were added Ruphos (187.5 mg, 402 μmol), Cs₂CO₃ (1.96 g, 6.03 mmol) and RuPhos Pd G3 (168.1 mg, 201 μmol), and the reaction was stirred for 3 h at 100° C. under N₂. The resulting mixture was concentrated under reduced pressure and purified by silica gel column chromatography (9% MeOH in DCM) to afford tert-butyl 4-(2,1,3-benzoxadiazol-4-yl)piperazine-1-carboxylate (A212, 0.45 g, 1.48 mmol). LCMS (ES, m/z): 327 [M+Na]⁺.

### Synthesis of Intermediate A213

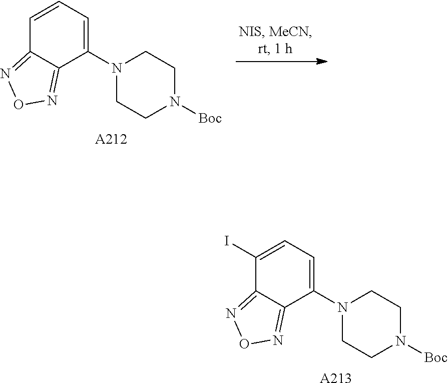

To a stirred solution of Intermediate A212 (0.43 g, 1.41 mmol) in MeCN (5 mL) was added NIS (317.8 mg, 1.41 mmol), and the reaction was stirred for 1 h at room temperature. The resulting mixture was diluted with water (5 mL) and extracted with EtOAc (3×5 mL). The combined organic layers were washed with brine (3×10 mL), dried over anhydrous Na₂SO₄, filtered, and concentrated under reduced pressure to afford tert-butyl 4-(4-iodo-2,1,3-benzoxadiazol-7-yl)piperazine-1-carboxylate (A213, 0.46 g, 1.07 mmol, 76% yield) as a solid. LCMS (ES, m/z): 431 [M+H]⁺.

### Synthesis of Intermediate A214

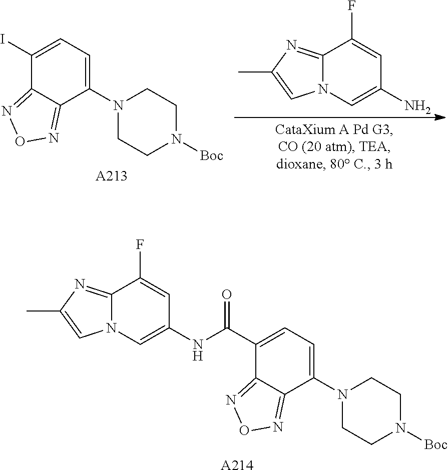

To a solution of Intermediate A213 (0.35 g, 813.5 μmol) and 8-fluoro-2-methyl-imidazo[1,2-a]pyridin-6-amine (161.2 mg, 976.2 μmol) in dioxane (5 mL) was added TEA (411.5 mg, 4.07 mmol, 566.9 μL) and cataCXium-A-Pd-G3 (118.4 mg, 162.7 μmol) in a pressure tank. The mixture was purged with nitrogen for 10 min and then was pressurized with carbon monoxide at 30 atm for 3 h. The reaction mixture was cooled to room temperature and filtered to remove insoluble solids. The filtrate was then concentrated under reduced pressure and purified by silica gel column chromatography (50% EtOAc in PE) to afford tert-butyl 4-[7-[(8-fluoro-2-methyl-imidazo[1,2-a]pyridin-6-yl)carbamoyl]-2, 1,3-benzoxadiazol-4-yl]piperazine-1-carboxylate (A214, 0.35 g, 706.4 μmol). LCMS (ES, m/z): 496 [M+H]⁺.

### Synthesis of Compound 263

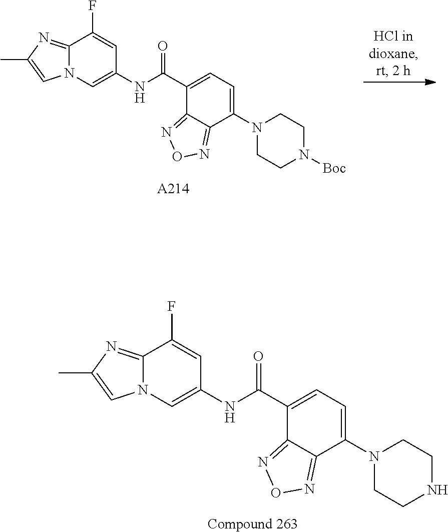

HCl (1 mL) (4 mol/L in dioxane) was added to a solution of Intermediate A214 (0.02 g, 40.36 μmol) in DCM (2 mL), stirred for 1 h at room temperature, then concentrated under reduced pressure and adjusted to pH 8 with saturated NaHCO₃ (aq.). The resulting mixture was diluted with water (5 mL) and extracted with DCM/MeOH (20/1) (2×10 mL). The combined organic layers were washed with water (20 mL), brine (20 mL) dried over anhydrous Na₂SO₄, and filtrate. The filtrate was concentrated under reduced pressure, and purified by reversed-phase flash chromatography (Condition 1, Gradient) to afford N-(8-fluoro-2-methyl-imidazo[1,2-a]pyridin-6-yl)-4-piperazin-1-yl-2,1,3-benzoxadiazole-7-carboxamide (Compound 263). LCMS (ES, m/z): 396 [M+H]⁺. ¹H NMR (300 MHz, DMSO-d₆) δ 10.02 (s, 1H), 9.12 (d, J=1.6 Hz, 1H), 8.09 (d, J=8.1 Hz, 1H), 7.92 (s, 1H), 7.33 (d, J=12.9 Hz, 1H), 6.67 (d, J=8.3 Hz, 1H), 3.78 (t, J=5.0 Hz, 4H), 2.91-2.90 (m, 4H), 2.35 (s, 3H).

### Example 42: Synthesis of Compound 232

### Synthesis of Intermediate A219

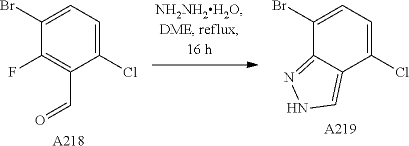

To a mixture of 3-bromo-6-chloro-2-fluoro-benzaldehyde (A218, 10 g, 42.11 mmol) in DME (100 mL) was added NH₂NH₂H₂O (13.5 g, 421.1 mmol), and the reaction was stirred for 16 h at 80° C. under N₂. The resulting mixture was concentrated under reduced pressure and purified by silica gel column chromatography (50% EtOAc in PE) to afford 7-bromo-4-chloro-2H-indazole (A219, 8 g, 34.56 mmol). LCMS (ES, m/z): 231 [M+H]⁺.

### Synthesis of Intermediate A220

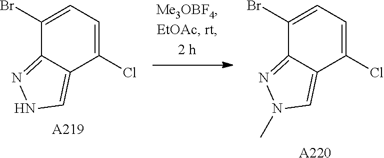

Intermediate A219 (3 g, 12.96 mmol) and Me₃OBF₄ (1.53 g, 12.96 mmol) in EtOAc (60 mL) were stirred for 2 h at room temperature, quenched by the addition of water (50 mL), and then extracted with EtOAc (2×50 mL). The combined organic layer was dried over anhydrous Na₂SO₄ and concentrated under reduced pressure to afford 7-bromo-4-chloro-2-methyl-indazole (A220, 2.5 g, 10.2 mmol). LCMS (ES, m/z): 245 [M+H]⁺.

### Synthesis of Intermediate A221

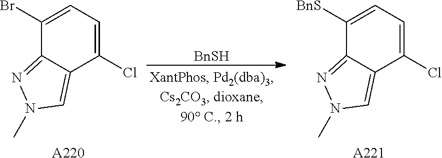

To a mixture of Intermediate A220 (1.5 g, 6.11 mmol) and BnSH (744.3 mg, 12.22 mmol) in dioxane (15 mL) were added Cs₂CO₃ (5.96 g, 18.33 mmol, 3.2 mL), Xantphos (707.0 mg, 1.22 mmol) and Pd₂(dba)₃ (559.5 mg, 611 μmol), and the reaction was stirred for 2 h at 90° C. The resulting mixture was concentrated under reduced pressure and purified by silica gel column chromatography (50% EtOAc in PE) to afford 7-benzylsulfanyl-4-chloro-2-methyl-indazole (A221, 1.5 g, 5.19 mmol). LCMS (ES, m/z): 289 [M+H]⁺.

### Synthesis of Intermediate A222

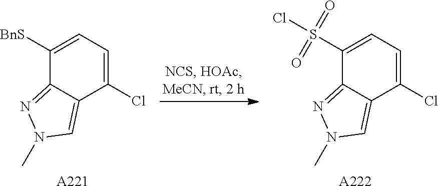

To a mixture of Intermediate A221 (500.0 mg, 1.73 mmol) in MeCN (25 mL) were added HOAc (0.5 mL), H₂O (0.2 mL), and NCS (184.9 mg, 1.39 mmol). The mixture was stirred for 2 h at room temperature, quenched by the addition of water (2×10 mL), and subsequently extracted with EtOAc (2×10 mL). Then the mixture was dried over anhydrous Na₂SO₄ and concentrated under reduced pressure to afford 4-chloro-2-methyl-indazole-7-sulfonyl chloride (A222, 300 mg, 1.13 mmol). LCMS (ES, m/z): 265 [M+H]⁺.

### Synthesis of Intermediate A223

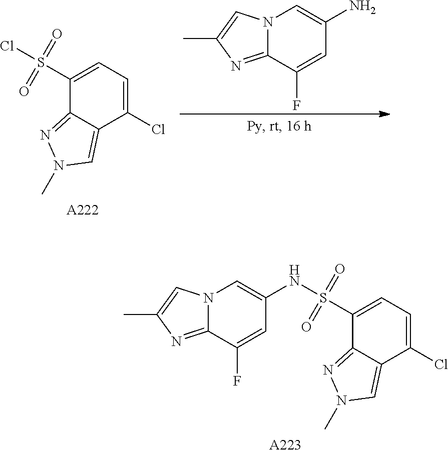

To a mixture of Intermediate A55 (0.3 g, 1.13 mmol) in Py (7.36 g, 93.1 mmol, 7.5 mL) was added 8-fluoro-2-methyl-imidazo[1,2-a]pyridin-6-amine (93.4 mg, 565.8 μmol). The resulting mixture was stirred for 16 h at room temperature, concentrated under reduced pressure, quenched by the addition of water (2×10 mL), and then extracted with EtOAc (2×10 mL). The resulting mixture was subsequently dried over anhydrous Na₂SO₄, concentrated under reduced pressure, purified by silica gel column chromatography (50% EtOAc in PE) to afford 4-chloro-N-(8-fluoro-2-methyl-imidazo[1,2-a]pyridin-6-yl)-2-methyl-indazole-7-sulfonamide (A56, 0.18 g, 457.1 μmol). LCMS (ES, m/z): 394 [M+H]⁺.

### Synthesis of Intermediate A224

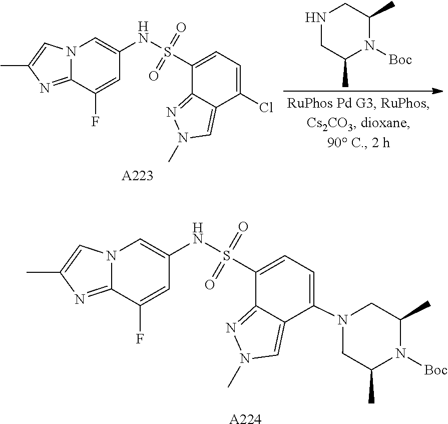

To a mixture of Intermediate A223 (0.15 g, 380.9 μmol) and tert-butyl (2R,6S)-2,6-dimethylpiperazine-1-carboxylate (97.9 mg, 457.1 μmol) in dioxane (3 mL) were added RuPhos (35.5 mg, 76.2 μmol), Cs₂CO₃ (248.2 mg, 761.8 μmol) and RuPhos Pd G3 (31.8 mg, 38.1 μmol), and the reaction was stirred for 2 h at 90° C. under N₂. The resulting mixture was concentrated under reduced pressure and purified by silica gel column chromatography (50% EtOAc in PE) to afford tert-butyl (2R,6S)-4-[7-[(8-fluoro-2-methyl-imidazo[1,2-a]pyridin-6-yl) sulfamoyl]-2-methyl-indazol-4-yl]-2,6-dimethyl-piperazine-1-carboxylate (A224, 0.06 g, 105 μmol). LCMS (ES, m/z): 572 [M+H]⁺.

### Synthesis of Compound 232

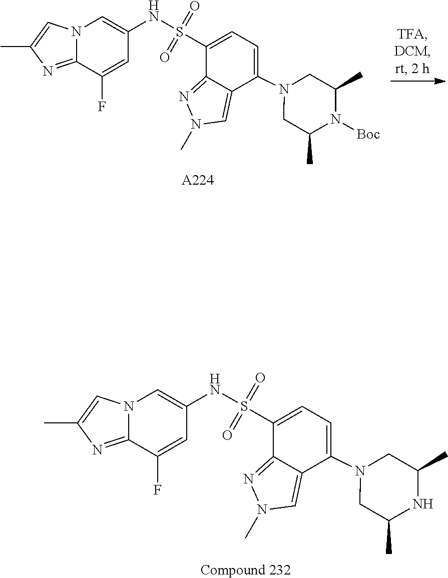

TFA (562.5 mg, 4.93 mmol, 0.75 mL) was added to solution of Intermediate A224 (60.0 mg, 105 μmol) in DCM (1.5 mL), and the reaction was stirred for 2 h at room temperature. The resulting mixture was concentrated under reduced pressure and purified by Prep-HPLC (Condition 2, Gradient 1) to afford 4-[(3R,5S)-3,5-dimethylpiperazin-1-yl]-N-(8-fluoro-2-methyl-imidazo[1,2-a]pyridin-6-yl)-2-methyl-indazole-7-sulfonamide (Compound 232, 13 mg, 27.57 μmol). LCMS (ES, m/z): 472 [M+H]⁺. ¹H NMR (400 MHz, Methanol-d₄) δ 8.59 (s, 1H), 8.34 (d, J=1.5 Hz, 1H), 7.89 (d, J=2.4 Hz, 1H), 7.85 (d, J=8.0 Hz, 1H), 7.36 (dd, J=11.2, 1.6 Hz, 1H), 6.50 (d, J=8.1 Hz, 1H), 4.24 (s, 3H), 4.02 (dd, J=13.7, 2.9 Hz, 2H), 3.61 (dqd, J=9.4, 6.2, 2.7 Hz, 2H), 2.94 (dd, J=13.7, 11.2 Hz, 2H), 2.47 (s, 3H), 1.42 (d, J=6.5 Hz, 6H).

### Example 43: Synthesis of Compound 237

### Synthesis of Intermediate A226

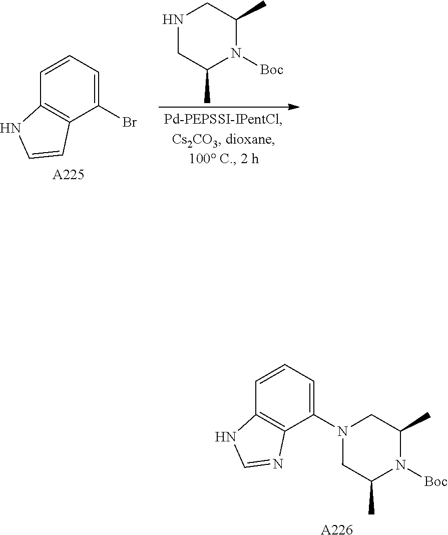

To a stirred solution of 4-bromo-1H-indole (A225, 500 mg, 2.55 mmol, 1 eq) and tert-butyl (2R,6S)-2,6-dimethylpiperazine-1-carboxylate (820 mg, 3.83 mmol, 1.5 eq) in dioxane (10 mL) were added Cs₂CO₃ (1662 mg, 5.1 mmol, 2 eq) and Pd-PEPPSI-IPentCl 2-methylpyridine (o-picoline (215 mg, 0.26 mmol, 0.1 eq), and the reaction was stirred for 12 h at 100° C. under N₂. The resulting mixture was concentrated under reduced pressure and purified by silica gel column chromatography (50% EtOAc in PE) to afford tert-butyl (2R,6S)-4-(1H-indol-4-yl)-2,6-dimethylpiperazine-1-carboxylate (A226, 220 mg). LCMS (ES, m/z): 330 [M+H]⁺.

### Synthesis of Intermediate A227

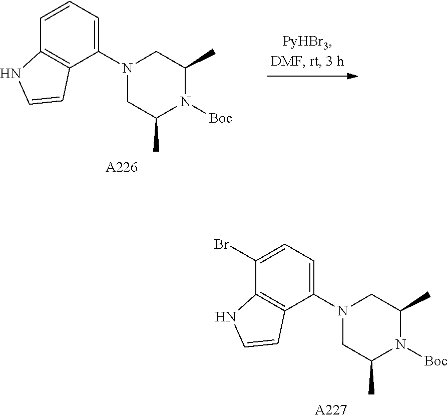 [SMILES: C[C@@H]1CN(c2cccc3[nH]ccc23)C[C@H](C)N1C(=O)OC(C)(C)C ; "A226" ; C[C@@H]1CN(c2ccc(Br)c3[nH]ccc23)C[C@H](C)N1C(=O)OC(C)(C)C ; "A227"]

A solution of Intermediate A226 (220 mg, 0.67 mmol, 1 eq) and pyridine tribromide (203 mg, 0.64 mmol, 0.95 eq) in DMF (4 mL) was stirred for 3 h at room temperature, quenched by the addition of water (10 mL) at room temperature, and subsequently extracted with EtOAc (3×5 mL). The combined organic layers were washed with brine (5 mL), dried over anhydrous Na₂SO₄, and filtered. The filtrate was concentrated under reduced pressure and purified by silica gel column chromatography (20% EtOAc in PE) to afford tert-butyl (2R,6S)-4-(7-bromo-1H-indol-4-yl)-2,6-dimethylpiperazine-1-carboxylate (A227, 100 mg) as a solid. LCMS (ES, m/z): 408 [M+H]⁺.

### Synthesis of Intermediate A228

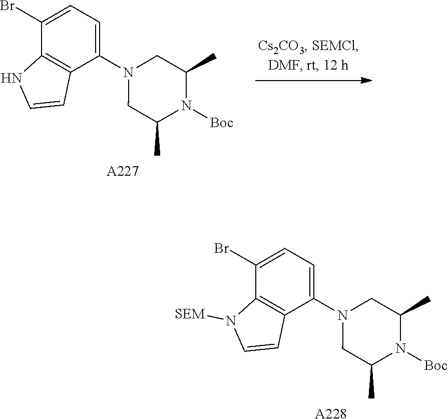 [SMILES: C[C@@H]1CN(c2ccc(Br)c3[nH]ccc23)C[C@H](C)N1C(=O)OC(C)(C)C ; "A227" ; C[C@@H]1CN(c2ccc(Br)c3c2ccn3COCC[Si](C)(C)C)C[C@H](C)N1C(=O)OC(C)(C)C ; "A228"]

To a stirred solution of Intermediate A227 (100 mg, 0.25 mmol, 1 eq) and Cs₂CO₃ (240 mg, 0.74 mmol, 3 eq) in DMF (2.5 mL) was added SEMCl (82 mg, 0.49 mmol, 2 eq) dropwise at 0° C. The resulting mixture was stirred for 12 h at room temperature, quenched by the addition of water (10 mL) at room temperature, and later extracted with EtOAc (3×5 mL). The combined organic layers were washed with brine (5 mL), dried over anhydrous Na₂SO₄, and filtered. The filtrate was concentrated under reduced pressure and purified by silica gel column chromatography (9% EtOAc in PE) to afford tert-butyl (2R,6S)-4-(7-bromo-1-([2-(trimethylsilyl)ethoxy]methyl)indol-4-yl)-2,6-dimethylpiperazine-1-carboxylate (A228, 75 mg). LCMS (ES, m/z): 538 [M+H]⁺.

### Synthesis of Intermediate A229

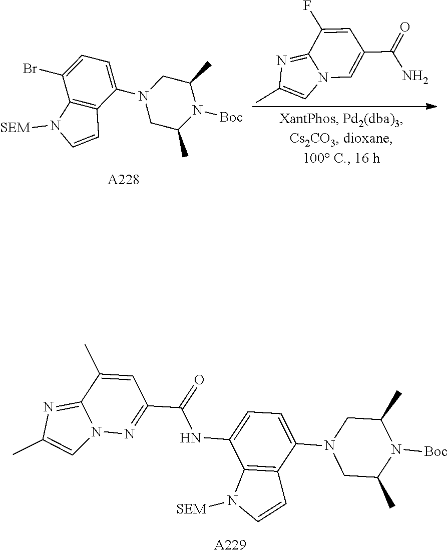

To a stirred solution of Intermediate A228 (75 mg, 0.14 mmol, 1 eq) and 2,8-dimethylimidazo[1,2-b]pyridazine-6-carboxamide (35 mg, 0.18 mmol, 1.3 eq) in dioxane (2 mL) were added Cs₂CO₃ (91 mg, 0.28 mmol, 2 eq), XantPhos (17 mg, 0.03 mmol, 0.2 eq) and Pd₂(dba)₃ (13 mg, 0.014 mmol, 0.1 eq), and the reaction was stirred for 16 h at 100° C. under N₂. The resulting mixture was concentrated under reduced pressure and purified by silica gel column chromatography (33% EtOAc in PE) to afford tert-butyl (2R,6S)-4-(7-(2,8-dimethylimidazo[1,2-b]pyridazine-6-amido)-1-([2-(trimethylsilyl)ethoxy]methyl)-indol-4-yl)-2,6-dimethylpiperazine-1-carboxylate (A229, 45 mg). LCMS (ES, m/z): 648 [M+H]⁺.

### Synthesis of Compound 237

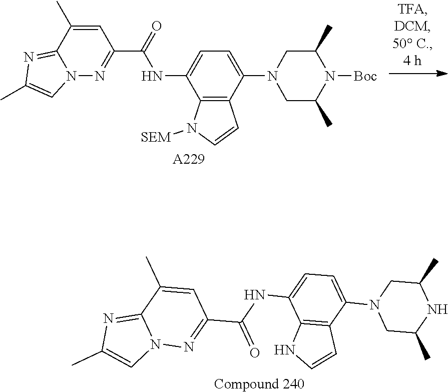

A solution of Intermediate A229 (45 mg, 0.07 mmol, 1 eq) and TFA (0.5 mL) in DCM (2 mL) was stirred for 4 h at 50° C. The resulting mixture was concentrated under reduced pressure, NH₃·H₂O (1 mL) was added and the reaction was stirred for additional 2 h at room temperature. The resulting mixture was concentrated under reduced pressure and purified by reversed-phase flash chromatography (Condition 1, Gradient 9) to afford N-(4-[(3R,5S)-3,5-dimethylpiperazin-1-yl]-1H-indol-7-yl)-2,8-dimethylimidazo[1,2-b]pyridazine-6-carboxamide (Compound 237, 3 mg). LCMS (ES, m/z): 418 [M+H]⁺. ¹H NMR (300 MHz, CDCl₃) δ 10.21 (s, 1H), 9.49 (s, 1H), 7.82 (d, J=15.0 Hz, 2H), 6.93 (d, J=7.8 Hz, 1H), 6.60 (s, 2H), 3.61 (d, J=11.3 Hz, 2H), 3.31 (s, 2H), 2.78 (s, 3H), 2.60 (s, 3H), 2.49 (s, 2H), 1.23 (s, 6H).

### Example 44: Synthesis of Compound 242

### Synthesis of Intermediate A231

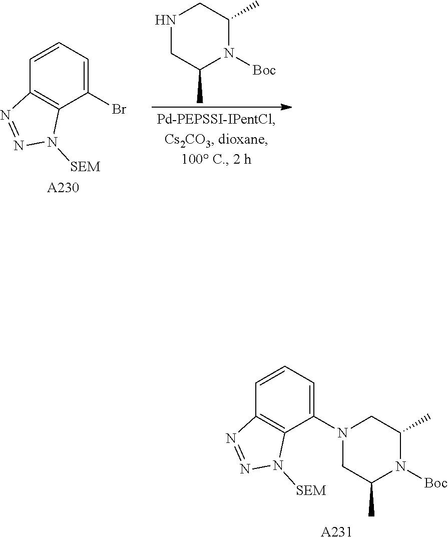

To a mixture of 2-[(7-bromobenzotriazol-1-yl)methoxy]ethyl-trimethyl-silane (A230, 0.5 g, 1.52 mmol) and tert-butyl(2S,6S)-2,6-dimethylpiperazine-1-carboxylate (391.7 mg, 1.83 mmol) were added Cs₂CO₃ (992.5 mg, 3.05 mmol) and Pd-PEPSSI-IPentCl (108.1 mg, 152.3 μmol), and the reaction was stirred for 2 h at 100° C. under N₂. The resulting mixture was concentrated under reduced pressure and purified by silica gel column chromatography (25% EtOAc in PE) to afford tert-butyl(2S,6S)-2,6-dimethyl-4-[3-(2-trimethylsilylethoxymethyl)-benzotriazol-4-yl]piperazine-1-carboxylate (A231, 0.45 g, 975 μmol). LCMS (ES, m/z): 462 [M+H]⁺.

### Synthesis of Intermediate A232

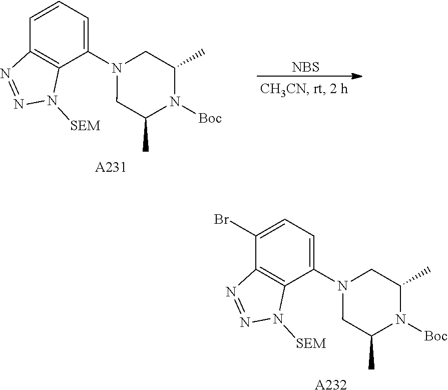

To a stirred solution of Intermediate A231 (0.50 g, 1.08 mmol) in MeCN (5 mL) was added 1-bromopyrrolidine-2,5-dione (154.2 mg, 866.4 μmol) at room temperature, stirred for 2 h, then diluted with water (20 mL) and extracted with EtOAc (2×20 mL). The combined organic layers were washed with brine (2×30 mL), dried over anhydrous Na₂SO₄, and filtered. The filtrate was concentrated under reduced pressure and purified by silica gel column chromatography (33% EtOAc in DCM) to afford tert-butyl(2S,6S)-4-[7-bromo-3-(2-trimethylsilylethoxymethyl)benzotriazol-4-yl]-2,6-dimethyl-piperazine-1-carboxylate (A232, 0.30 g, 555 μmol). LCMS (ES, m/z): 540 [M+H]⁺.

### Synthesis of Intermediate A233

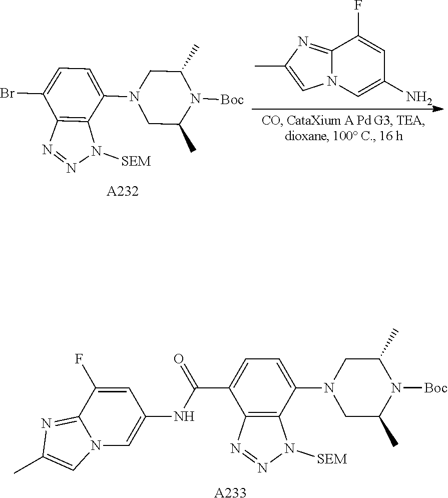

To a stirred solution of Intermediate A232 (150.0 mg, 277.5 μmol) and 8-fluoro-2-methyl-imidazo[1,2-a]pyridin-6-amine (55 mg, 333 μmol) in dioxane (5 mL) was added CataXium A Pd G3 (20.2 mg, 27.75 μmol) and TEA (140.4 mg, 1.39 mmol, 193.4 μL), and the reaction was stirred for 16 h at 100° C. The resulting mixture was concentrated under reduced pressure and purified by silica gel column chromatography (9% MeOH in DCM) to afford tert-butyl(2S,6S)-4-[7-[(8-fluoro-2-methyl-imidazo[1,2-a]pyridin-6-yl)carbamoyl]-3-(2-trimethylsilylethoxymethyl)benzotriazol-4-yl]-2,6-dimethyl-piperazine-1-carboxylate (A233, 80 mg, 122.5 μmol). LCMS (ES, m/z): 653 [M+H]⁺.

### Synthesis of Compound 242

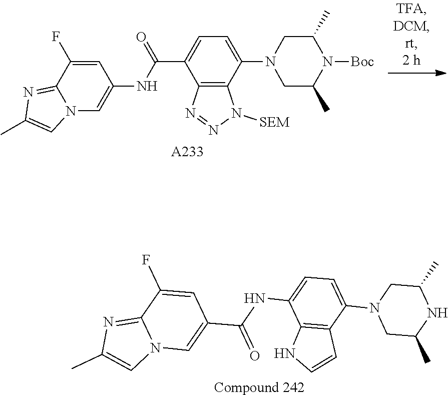

To a stirred solution of Intermediate A233 (80 mg, 122.5 μmol) in DCM (2.5 mL) was added TFA (0.75 mL), and the reaction was stirred for 2 h at room temperature. The resulting mixture was concentrated under reduced pressure and purified by Prep-HPLC (Condition 2, Gradient 2) to afford 7-[(3S,5S)-3,5-dimethylpiperazin-1-yl]-N-(8-fluoro-2-methyl-imidazo[1,2-a]pyridin-6-yl)-1H-benzotriazole-4-carboxamide (Compound 242, 18.4 mg, 43.6 μmol). LCMS (ES, m/z): 423 [M+H]⁺. ¹H NMR (400 MHz, DMSO-d₆) δ 15.78 (s, 1H), 10.38 (s, 1H), 9.10 (s, 1H), 8.95 (s, 2H), 8.14 (d, J=8.4 Hz, 1H), 8.01 (s, 1H), 7.55 (d, J=12.4 Hz, 1H), 6.83 (d, J=8.4 Hz, 1H), 4.14 (d, J=6.6 Hz, 4H), 3.87-3.81 (m, 3H), 2.39 (s, 3H), 1.37 (d, J=6.6 Hz, 6H).

### Example 45: Synthesis of Compound 243

### Synthesis of Intermediate A234

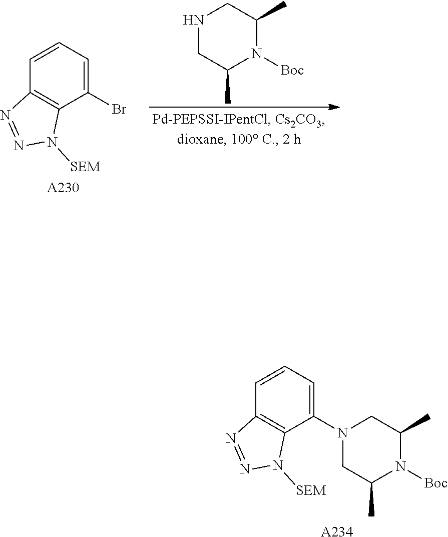

To a stirred solution of Intermediate A230 (0.3 g, 913.9 μmol) and tert-butyl (2R,6S)-2,6-dimethylpiperazine-1-carboxylate (235.0 mg, 1.10 mmol) in dioxane (2 mL) were added Cs₂CO₃ (595.5 mg, 1.83 mmol) and Pd-PEPSSI-IPentCl (72.3 mg, 91.4 μmol), and the reaction was stirred for 2 h at 100° C. under N₂. The resulting mixture was concentrated under reduced pressure and purified by silica gel column chromatography (80% EtOAc in PE) to afford tert-butyl (2R,6S)-2,6-dimethyl-4-[3-(2-trimethylsilylethoxymethyl)-benzotriazol-4-yl]piperazine-1-carboxylate (A234, 0.28 g, 606.5 μmol). LCMS (ES, m/z): 462 [M+H]⁺.

### Synthesis of Intermediate A235

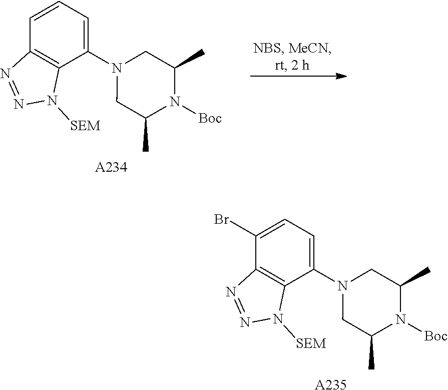

To a stirred solution of Intermediate A234 (0.28 g, 606.5 μmol) in MeCN (5 mL) was added NBS (0.11 g, 618.0 μmol) at room temperature. The resulting mixture was stirred for 2 h at room temperature under N₂, diluted with water (5 mL), and subsequently extracted with EtOAc (3×10 mL). The combined organic layers were washed with brine (3×20 mL), dried over anhydrous Na₂SO₄, filtered, and concentrated under reduced pressure to afford product tert-butyl(2R,6S)-4-[7-bromo-3-(2-trimethylsilylethoxymethyl)benzotriazol-4-yl]-2,6-dimethyl-piperazine-1-carboxylate (A235, 0.18 g, 333 μmol). LCMS (ES, m/z): 541 [M+H]⁺.

### Synthesis of Intermediate A236

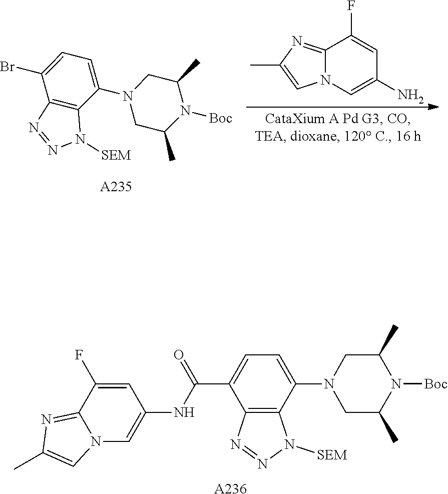

To a solution of Intermediate A235 (0.18 g, 333 μmol) and 8-fluoro-2-methyl-imidazo[1,2-a]pyridin-6-amine (66 mg, 399.6 μmol) in 5 mL of dioxane was added CataXium A Pd G3 (51.8 mg, 66.6 μmol) and TEA (168.4 mg, 1.66 mmol, 232.1 μL) in a pressure tank. The mixture was purged with nitrogen for 1 h and then was pressurized to 20 atm with carbon monoxide at 120° C. for 16 h. The reaction mixture was cooled to room temperature and filtered to remove insoluble solids. The filtrate was concentrated under reduced pressure and purified by silica gel column chromatography (91% MeOH in DCM) to afford tert-butyl (2R,6S)-4-[7-[(8-fluoro-2-methyl-imidazo[1,2-a]pyridin-6-yl)carbamoyl]-3-(2-trimethylsilylethoxymethyl)-benzotriazol-4-yl]-2,6-dimethyl-piperazine-1-carboxylate (A236, 0.08 g, 122.5 μmol). LCMS (ES, m/z): 653 [M+H]⁺.

### Synthesis of Compound 243

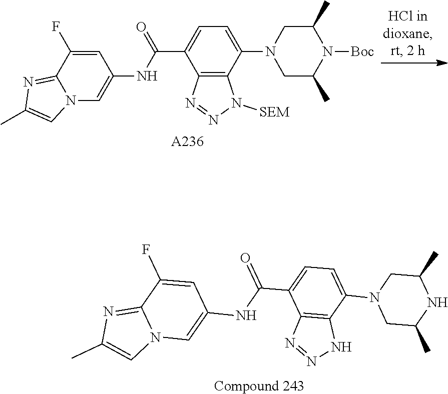

To a stirred solution of Intermediate A236 (0.08 g, 122.5 μmol) in DCM (3 mL) was added HCl in dioxane (1 mL, 4 M), and the reaction was stirred for 2 h at room temperature. The resulting mixture was concentrated under reduced pressure and purified by reversed-phase flash chromatography (Condition 3, Gradient 1) to afford product 7-((3S,5R)-3,5-dimethylpiperazin-1-yl)-N-(8-fluoro-2-methylimidazo[1,2-a]pyridin-6-yl)-1H-benzo[d][1,2,3]-triazole-4-carboxamide hydrochloride (Compound 243, 17 mg, 37.0 μmol). LCMS (ES, m/z): 423 [M+H]⁺. ¹H NMR (300 MHz, DMSO-d₆) δ 15.79 (s, 1H), 10.80 (s, 1H), 9.51 (s, 1H), 9.29 (s, 1H), 9.14 (s, 1H), 8.31 (s, 1H), 8.20 (s, 1H), 8.01 (s, 1H), 6.87 (d, J=8.4 Hz, 1H), 5.05 (d, J=13.4 Hz, 2H), 3.21 (t, J=12.6 Hz, 2H), 2.46 (s, 3H), 1.38 (d, J=6.4 Hz, 6H).

### Example 46: Synthesis of Compound 250

### Synthesis of Intermediate A245

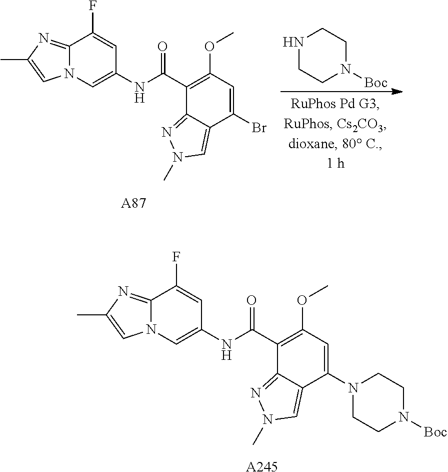

To a solution of 4-bromo-N-(8-fluoro-2-methyl-imidazo[1,2-a]pyridin-6-yl)-6-methoxy-2-methyl-indazole-7-carboxamide (A87, 0.1 g, 231.4 μmol) and tert-butyl piperazine-1-carboxylate (43 mg, 231.4 μmol) in dioxane (2 mL) were added Cs₂CO₃ (226 mg, 694.1 μmol), RuPhos Pd G3 (19 mg, 23.13 μmol) and Ruphos (22 mg, 46.27 μmol), and the reaction was stirred for 1 h at 80° C. under N₂. The resulting mixture was concentrated under reduced pressure and purified by reversed-phase flash chromatography (Condition 1, Gradient 6) to afford tert-butyl 4-[7-[(8-fluoro-2-methyl-imidazo[1,2-a]pyridin-6-yl)carbamoyl]-6-methoxy-2-methyl-indazol-4-yl]piperazine-1-carboxylate (A245, 62 mg, 115.3 μmol). LCMS (ES, m/z): 538 [M+H]⁺.

### Synthesis of Compound 250

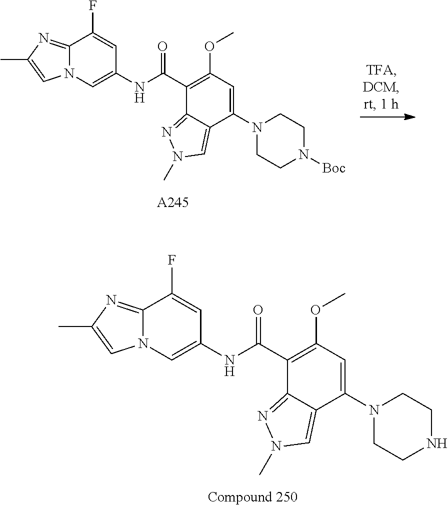

To a stirred solution of Intermediate A245 (0.05 g, 93.01 μmol) in DCM (2 mL) was added TFA (0.2 mL), and the reaction was stirred for 1 h at room temperature. The resulting mixture was basified to pH 8 with NH₃ (g) in MeOH, concentrated under reduced pressure, and purified by reversed-phase flash chromatography (Condition 1, Gradient 11) to afford N-(8-fluoro-2-methyl-imidazo[1,2-a]pyridin-6-yl)-6-methoxy-2-methyl-4-piperazin-1-yl-indazole-7-carboxamide (Compound 250, 27 mg, 61.7 μmol). LCMS (ES, m/z): 438 [M+H]⁺. ¹H NMR (300 MHz, DMSO-d₆) δ 10.49 (s, 1H), 9.23 (d, J=1.6 Hz, 1H), 8.51 (s, 1H), 7.89 (d, J=3.0 Hz, 1H), 7.21-7.11 (m, 1H), 6.22 (s, 1H), 4.10 (s, 3H), 3.87 (s, 3H), 3.26 (t, J=4.9 Hz, 4H), 2.91 (t, J=4.9 Hz, 4H), 2.34 (s, 3H).

### Example 47: Synthesis of Compound 251

### Synthesis of Intermediate A246

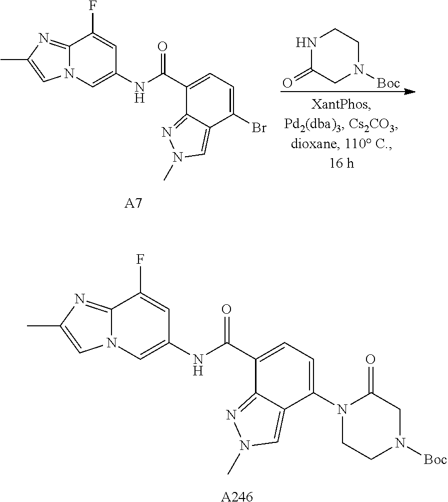

To a stirred mixture of Intermediate A7 (150.0 mg, 372.9 μmol) and tert-butyl 3-oxopiperazine-1-carboxylate (89.6 mg, 447.5 μmol) in dioxane (5 mL) were added Xantphos (21.5 mg, 37.29 μmol), Pd₂(dba)₃ (68.3 mg, 74.59 μmol) and Cs₂CO₃ (243.0 mg, 745.9 μmol), and the reaction was stirred for 16 h at 110° C. under N₂. The resulting mixture was concentrated under reduced pressure and purified by silica gel column chromatography (9% MeOH in DCM) to afford tert-butyl 4-[7-[(8-fluoro-2-methyl-imidazo[1,2-a]pyridin-6-yl)carbamoyl]-2-methyl-indazol-4-yl]-3-oxo-piperazine-1-carboxylate (A246, 80.0 mg, 153.4 μmol). LCMS (ES, m/z): 522 [M+H]⁺.

### Synthesis of Compound 251

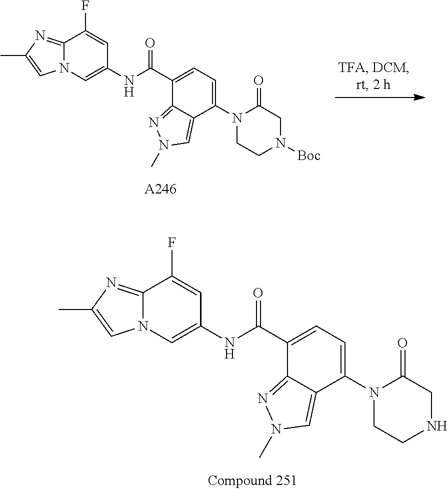

TFA (1.49 g, 13.07 mmol, 1 mL) was added to a solution of Intermediate A246 (80 mg, 153.4 μmol) in DCM (2 mL) and stirred for 2 h at room temperature. The crude product was purified by Prep-HPLC (Condition 2, Gradient 3) to afford N-(8-fluoro-2-methylimidazo[1,2-a]-pyridin-6-yl)-2-methyl-4-(2-oxopiperazin-1-yl)-2H-indazole-7-carboxamide 2,2,2-trifluoroacetate (Compound 251, 12.0 mg, 28.5 μmol). LCMS (ES, m/z): 422 [M+H]⁺. ¹H NMR (400 MHz, DMSO-d₆) δ 11.15 (s, 1H), 9.42 (s, 2H), 9.37 (s, 1H), 8.53 (s, 1H), 8.15 (d, J=7.6 Hz, 1H), 8.06 (s, 1H), 7.62 (d, J=12.2 Hz, 1H), 7.23 (d, J=7.6 Hz, 1H), 4.37 (s, 3H), 4.03-3.94 (m, 4H), 3.64 (t, J=5.8 Hz, 2H), 2.41 (s, 3H).

### Example 48: Synthesis of A248

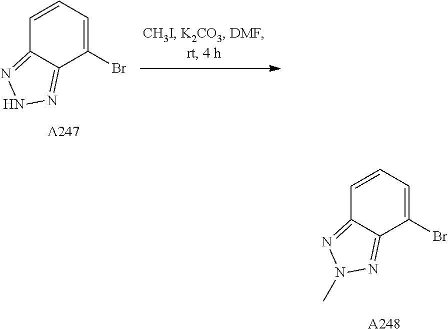

To a stirred solution of 4-bromo-2H-1,2,3-benzotriazole (A247, 3 g, 15.15 mmol, 1 eq) and K₂CO₃ (4.19 g, 30.3 mmol, 2 eq) in DMF (30 mL) was added Mel (3.23 g, 22.73 mmol, 1.5 eq) dropwise at room temperature. The resulting mixture was stirred for 4 h, dissolved in water (50 mL), and subsequently extracted with EtOAc (3×50 mL). The combined organic layers were washed with brine (3×100 mL), dried over anhydrous Na₂SO₄, and filtered. The filtrate was concentrated under reduced pressure and purified by silica gel column chromatography (9% EtOAc in PE) to afford 4-bromo-2-methyl-1,2,3-benzotriazole (A248, 850 mg), 7-bromo-1-methyl-1,2,3-benzotriazole (840 mg) and 4-bromo-1-methyl-1,2,3-benzotriazole (750 mg). LCMS (ES, m/z): 212 [M+H]⁺.

### Example 49: Synthesis of Compound 257

### Synthesis of Intermediate A255

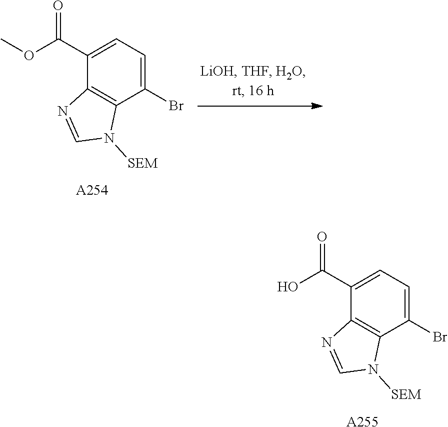 [SMILES: COC(=O)c1ccc(Br)c2c1ncn2COCC[Si](C)(C)C ; "A254" ; C[Si](C)(C)CCOCn1cnc2c(C(=O)O)ccc(Br)c21 ; "A255"]

To a stirred mixture of methyl 7-bromo-1-(2-trimethylsilylethoxymethyl)benzimidazole-4-carboxylate (A254, 0.5 g, 1.30 mmol) in THF (5 mL) and H₂O (2 mL) was added LiOH (155.3 mg, 6.49 mmol), and the reaction was stirred for 16 h at room temperature. The resulting mixture was neutralized to pH 5 with 1 N of HCl (aq.), diluted with water (10 mL), and extracted with EtOAc (2×20 mL). The combined organic layers were washed with water (2×40 mL), brine (40 mL), dried over anhydrous Na₂SO₄, filtered, and concentrated under reduced pressure to afford 7-bromo-1-(2-trimethylsilylethoxymethyl)benzimidazole-4-carboxylic acid (A255, 0.43 g, 1.16 mmol). LCMS (ES, m/z): 371 [M+H]⁺.

### Synthesis of Intermediate A256

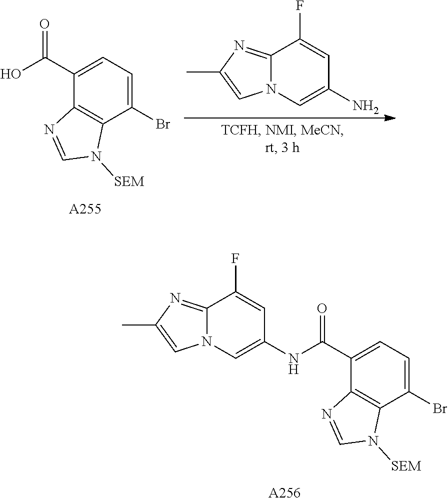 [SMILES: C[Si](C)(C)CCOCn1cnc2c(C(=O)O)ccc(Br)c21 ; Cc1cn2cc(N)cc(F)c2n1 ; "A255" ; Cc1cn2cc(NC(=O)c3ccc(Br)c4c3ncn4COCC[Si](C)(C)C)cc(F)c2n1 ; "A256"]

To a stirred mixture of Intermediate A255 (0.43 g, 1.16 mmol) and 8-fluoro-2-methyl-imidazo[1,2-a]pyridin-6-amine (229.5 mg, 1.39 mmol) in MeCN (5 mL) were added TCFH (487.4 mg, 1.74 mmol) and NMI (285.3 mg, 3.47 mmol, 275.6 μL), and the reaction was stirred for 16 h at room temperature. The resulting mixture was diluted with water (3 mL). The solid was collected by filtration, washed with water (2 mL) and MTBE (2 mL), and dried to afford 7-bromo-N-(8-fluoro-2-methyl-imidazo[1,2-a]pyridin-6-yl)-1-(2-trimethylsilylethoxymethyl)-benzimidazole-4-carboxamide (A256, 0.33 g, 636.5 μmol). LCMS (ES, m/z): 518 [M+H]⁺.

### Synthesis of Intermediate A257

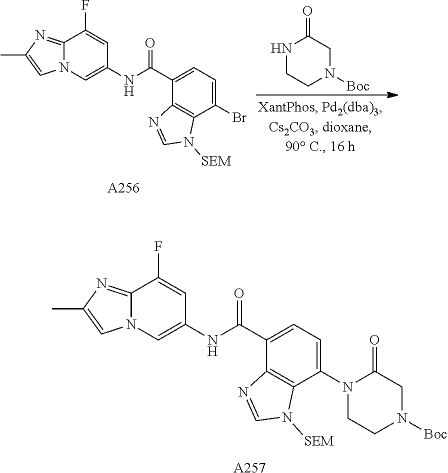 [SMILES: Cc1cn2cc(NC(=O)c3ccc(Br)c4c3ncn4COCC[Si](C)(C)C)cc(F)c2n1 ; CC(C)(C)OC(=O)N1CCNC(=O)C1 ; "A256" ; Cc1cn2cc(NC(=O)c3ccc(N4CCN(C(=O)OC(C)(C)C)CC4=O)c4c3ncn4COCC[Si](C)(C)C)cc(F)c2n1 ; "A257"]

To a stirred mixture of Intermediate A256 (0.2 g, 385.8 μmol) and tert-butyl 3-oxopiperazin-4-ium-1-carboxylate (194.1 mg, 964.4 μmol) in dioxane (4 mL) were added Cs₂CO₃ (376.1 mg, 1.16 mmol), XantPhos (44.6 mg, 77.15 μmol) and Pd₂(dba)₃ (35.3 mg, 38.6 μmol) at room temperature under N₂. The resulting mixture was stirred for 16 h at 90° C., allowed to cool to room temperature, diluted with water (10 mL), and subsequently extracted with EtOAc (2×10 mL). The combined organic layers were washed with water (2×20 mL), brine (20 mL), dried over anhydrous Na₂SO₄, and filtered. The filtrate was concentrated under reduced pressure and purified by silica gel column chromatography (89% EtOAc in PE) to afford tert-butyl 4-[7-[(8-fluoro-2-methyl-imidazo[1,2-a]pyridin-6-yl)carbamoyl]-3-(2-trimethylsilylethoxy-methyl)benzimidazol-4-yl]-3-oxo-piperazine-1-carboxylate (A257, 0.2 g, 313.6 μmol). LCMS (ES, m/z): 638 [M+H]⁺.

### Synthesis of Compound 257

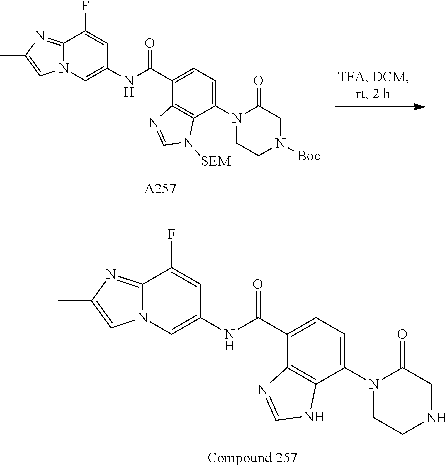 [SMILES: Cc1cn2cc(NC(=O)c3ccc(N4CCN(C(=O)OC(C)(C)C)CC4=O)c4c3ncn4COCC[Si](C)(C)C)cc(F)c2n1 ; "A257" ; Cc1cn2cc(NC(=O)c3ccc(N4CCNCC4=O)c4[nH]cnc34)cc(F)c2n1]

TFA (1 mL) was added to a solution of Intermediate A257 (0.2 g, 313.6 μmol) in DCM (2 mL), stirred for 2 h at room temperature. The resulting mixture was concentrated under reduced pressure and adjusted to pH 8 with saturated NaHCO₃ (aq.). The resulting mixture was diluted with water (5 mL) and extracted with DCM/MeOH (20/1) (2×10 mL). The combined organic layers were washed with water (20 mL), brine (20 mL), dried over anhydrous Na₂SO₄, and filtered. The filtrate was concentrated under reduced pressure and purified by reversed-phase flash chromatography (Condition 1, Gradient) to afford N-(8-fluoro-2-methyl-imidazo[1,2-a]-pyridin-6-yl)-7-(2-oxopiperazin-1-yl)-1H-benzimidazole-4-carboxamide (Compound 257, 0.02 g, 49.1 μmol). LCMS (ES, m/z): 408 [M+H]⁺. ¹H NMR (300 MHz, DMSO-d₆) δ 9.09 (t, J=1.9 Hz, 1H), 7.95-7.87 (m, 2H), 7.61 (dd, J=8.1, 5.6 Hz, 1H), 7.37-7.26 (m, 1H), 7.24 (s, 1H), 6.88 (dd, J=10.9, 8.0 Hz, 1H), 4.07-3.95 (m, 2H), 3.45-3.35 (m, 2H), 3.10-3.05 (m, 2H), 2.37 (s, 3H).

### Example 50: Synthesis of Compound 197

### Synthesis of Intermediate A258

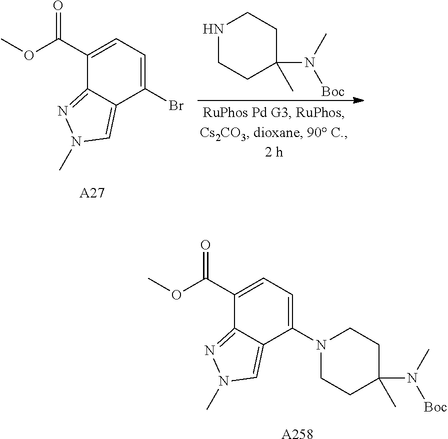

To a stirred mixture of Intermediate A27 (0.3 g, 1.11 mmol, 1 eq) and tert-butyl methyl (4-methylpiperidin-4-yl)carbamate (305 mg, 1.34 mmol, 1.2 eq) in dioxane (6 mL) were added Cs₂CO₃ (1.1 g, 3.34 mmol, 3 eq), Ruphos (104 mg, 223 μmol, 0.2 eq) and RuPhos Pd G3 (93 mg, 111.5 μmol, 0.1 eq), and the reaction was stirred for 2 h at 90° C. under N₂. The resulting mixture was concentrated under reduced pressure and purified by silica gel column chromatography (50% EtOAc in PE) to afford methyl 4-[4-[tert-butoxycarbonyl(methyl)amino]-4-methyl-1-piperidyl]-2-methyl-indazole-7-carboxylate (A258, 310 mg, 744.3 μmol). LCMS (ES, m/z): 417 [M+H]⁺.

### Synthesis of Intermediate A259

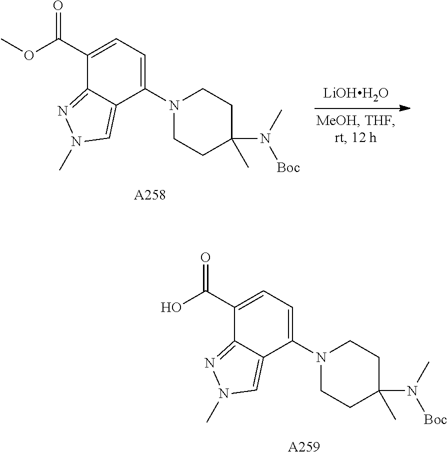

To a stirred mixture of Intermediate A258 (0.31 g, 744.3 μmol, 1 eq) in THF (3 mL) and MeOH (3 mL) was added a solution of LiOH (78 mg, 1.86 mmol, 2.5 eq) in H₂O (2 mL) at room temperature. The resulting mixture was stirred for 12 h and then neutralized to pH 5 with 1 N of HCl (aq.). The resulting solids were collected by filtration, washed with water (4 mL), and the solid was dried to afford 4-[4-[tert-butoxycarbonyl(methyl)amino]-4-methyl-1-piperidyl]-2-methyl-indazole-7-carboxylic acid (A259, 290 mg). LCMS (ES, m/z): 403 [M+H]⁺.

### Synthesis of Intermediate A260

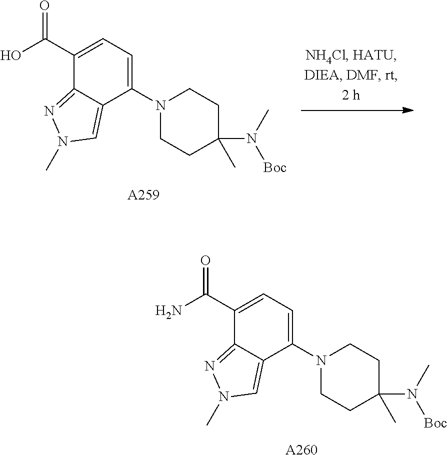 [SMILES: CN(C(=O)OC(C)(C)C)C1(C)CCN(c2ccc(C(=O)O)c3nn(C)cc23)CC1 ; "A259" ; CN(C(=O)OC(C)(C)C)C1(C)CCN(c2ccc(C(N)=O)c3nn(C)cc23)CC1 ; "A260"]

To a stirred mixture of Intermediate A259 (290 mg, 720.5 μmol, 1 eq) and NH₄Cl (77 mg, 1.44 mmol, 2 eq) in DMF (8 mL) were added DIEA (186 mg, 1.44 mmol, 2 eq) and HATU (356 mg, 936.7 mmol, 1.3 eq) at room temperature. The resulting mixture was stirred for 2 h, diluted with water (6 mL), and subsequently extracted with EtOAc (6×3 mL). The combined organic layers were washed with water (2×5 mL), brine (2×5 mL), dried over anhydrous Na₂SO₄, and filtered. The filtrate was concentrated under reduced pressure and purified by silica gel column chromatography (50% EtOAc in PE) to afford tert-butyl N-[1-(7-carbamoyl-2-methyl-indazol-4-yl)-4-methyl-4-piperidyl]-N-methyl-carbamate (A260, 200 mg). LCMS (ES, m/z): 403 [M+H].

### Synthesis of Intermediate A261

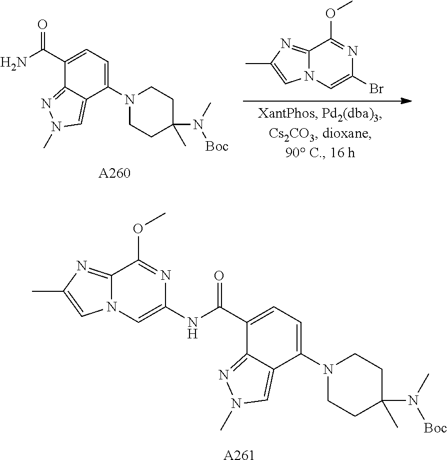

To a stirred mixture of Intermediate A260 (290 mg, 722.3 μmol, 1 eq) and 6-bromo-8-methoxy-2-methyl-imidazo[1,2-a]pyrazine (227 mg, 939 μmol, 1.3 eq) in dioxane (5 mL) were added Cs₂CO₃ (706 mg, 2.17 mmol, 3 eq), XantPhos (84 mg, 144.5 μmol, 0.2 eq) and Pd₂(dba)₃ (66 mg, 72.23 μmol, 1 eq), and the reaction was stirred for 16 h at 90° C. under N₂. The resulting mixture was allowed to cool to room temperature, diluted with water (20 mL), and extracted with EtOAc (2×10 mL). The combined organic layers were washed with brine (30 mL), dried over anhydrous Na₂SO₄, and filtered. The filtrate was concentrated under reduced pressure and purified by silica gel column chromatography (9% MeOH in DCM) to afford tert-butyl N-[1-[7-[(8-methoxy-2-methyl-imidazo[1,2-a]pyrazin-6-yl)carbamoyl]-2-methyl-indazol-4-yl]-4-methyl-4-piperidyl]-N-methyl-carbamate (A261, 150 mg, 266.6 μmol). LCMS (ES, m/z): 563 [M+H]⁺.

### Synthesis of Compound 197

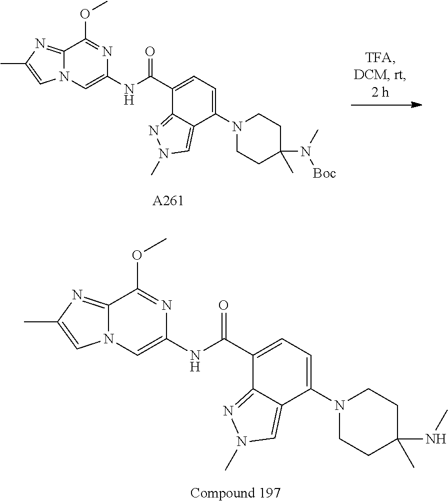

To a solution of tert-butyl N-[1-[7-[(8-methoxy-2-methyl-imidazo[1,2-a]pyrazin-6-yl)carbamoyl]-2-methyl-indazol-4-yl]-4-methyl-4-piperidyl]-N-methyl-carbamate (100 mg, 168.5 μmol, 1 eq) in DCM (3 mL) was added TFA (1 mL). The reaction was stirred for 2 h at room temperature. The resulting mixture was concentrated under reduced pressure and purified by reversed-phase flash chromatography (Condition 3, Gradient 11) to afford N-(8-methoxy-2-methylimidazo[1,2-a]pyrazin-6-yl)-2-methyl-4-(4-methyl-4-(methylamino)piperidin-1-yl)-2H-indazole-7-carboxamide (Compound 197, 50 mg, 61% yield) as a solid. LCMS (ES, m/z): 463 [M+H]⁺. ¹H NMR (300 MHz, DMSO-d₆) δ 11.27 (s, 1H), 9.01 (s, 1H), 8.78 (s, 1H), 7.99 (d, J=8.1 Hz, 1H), 7.92 (s, 1H), 6.48 (d, J=8.3 Hz, 1H), 4.26 (s, 3H), 4.11 (s, 3H), 3.50 (t, J=5.6 Hz, 4H), 2.34 (s, 3H), 2.22 (s, 3H), 1.77-1.48 (m, 4H), 1.09 (s, 3H).

An analogous method was followed to obtain the following compounds.

| | Starting | |
|---|---|---|
| Compound | Materials | Characterization |
| 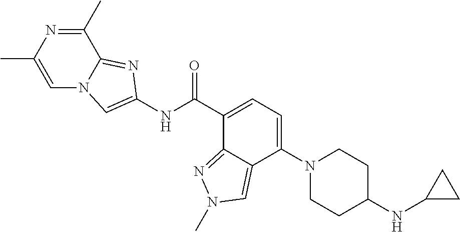 [SMILES: Cc1cn2cc(NC(=O)c3ccc(N4CCC(NC5CC5)CC4)c4cn(C)nc34)nc2c(C)n1] Compound 297 | 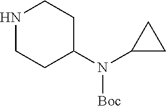 [SMILES: CC(C)(C)OC(=O)N(C1CCNCC1)C1CC1] 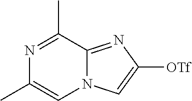 [SMILES: Cc1cn2cc(OS(=O)(=O)C(F)(F)F)nc2c(C)n1] | Modification: Step 1 was ran for 6 h. Step 2 was ran for 6 h. Step 3 was ran for 4 h. Step 4 was ran for 6 h. The residue was purified by reversed-phase flash chromatography (Condition 3, Gradient 11) to afford 4-[4- (cyclopropylamino)-1-piperidyl]- N-(6,8-dimethylimidazo[1,2-a] pyrazin-2-yl)-2-methyl-indazole- 7-carboxamide (Compound 297, 40 mg, 87.2 umol, 49% yield) as a solid. LCMS (ES, m/z): 459 [M + H]⁺. ¹H NMR (300 MHz, DMSO-d₆) δ |
| | | 11.44 (s, 1H), 8.79 (s, 1H), 8.32 (d, |
| | | J = 2.3 Hz, 2H), 8.03 (d, J = 8.1 Hz, |
| | | 1H), 6.50 (d, J = 8.3 Hz, 1H), 4.31 |
| | | (s, 3H), 3.90 (d, J = 12.8 Hz, 2H), |
| | | 3.08 (t, J = 11.5 Hz, 2H), 2.76 |
| | | (s, 1H), 2.70 (s, 3H), 2.41- |
| | | 2.35 (m, 3H), 2.13 (tt, J = 6.8, |
| | | 3.6 Hz, 1H), 2.01 (d, J = 12.8 Hz, |
| | | 2H), 1.49 (q, J = 9.6 Hz, 2H), |
| | | 0.40 (dt, J = 6.2, 3.0 Hz, 2H), |
| | | 0.24 (p, J = 3.9 Hz, 2H). |

### Example 51: Synthesis of Compound 268, 275, 277

### Synthesis of B1

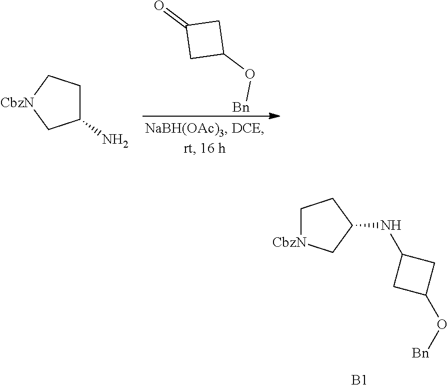 [SMILES: N[C@H]1CCN(C(=O)OCc2ccccc2)C1 ; O=C1CC(OCc2ccccc2)C1 ; O=C(OCc1ccccc1)N1CC[C@H](NC2CC(OCc3ccccc3)C2)C1 ; "B1"]

To a solution of benzyl (3S)-3-aminopyrrolidine-1-carboxylate (10 g, 45.4 mmol) in DCM (100 mL) was added 3-benzyloxycyclobutanone (7.27 g, 41.3 mmol), and the resulting mixture was stirred for 1 h at room temperature. Followed by addition of NaBH(OAc)₃ (26.2 g, 123.8 mmol), and the reaction was stirred for 16 h at rt. The resulting mixture was diluted with water (200 mL) and extracted with DCM (2×150 mL). The combined organic layers were washed with brine (2×200 mL), dried over anhydrous Na₂SO₄, and filtered. The filtrate was concentrated under reduced pressure and purified by silica gel column chromatography (67% EtOAc in PE) to afford benzyl (3S)-3-[(3-benzyloxycyclobutyl)amino]pyrrolidine-1-carboxylate (B1, 8.5 g, 22.3 mmol, 54% yield) as an oil. LCMS (ES, m/z): 381 [M+H]⁺.

Synthesis of B2

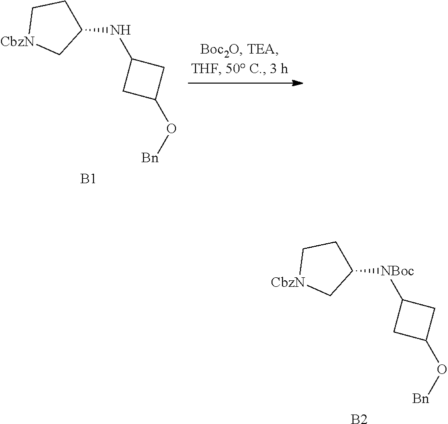 [SMILES: O=C(OCc1ccccc1)N1CC[C@H](NC2CC(OCc3ccccc3)C2)C1 ; "B1" ; CC(C)(C)OC(=O)N(C1CC(OCc2ccccc2)C1)[C@H]1CCN(C(=O)OCc2ccccc2)C1 ; "B2"]

To a solution of benzyl (3S)-3-[(3-benzyloxycyclobutyl)amino]pyrrolidine-1-carboxylate (B1, 8.6 g, 22.6 mmol) in THF (90 mL) were added TEA (6.86 g, 67.8 mmol, 9.5 mL) and Boc₂O (7.40 g, 33.9 mmol, 7.8 mL) at room temperature. The mixture was stirred for 3 h at 50° C. The resulting mixture was diluted with water (200 mL) and extracted with EtOAc (2×150 mL). The combined organic layers were washed with brine (200 mL), dried over anhydrous Na₂SO₄, and filtered. The filtrate was concentrated under reduced pressure and purified by silica gel column chromatography (33% EtOAc in PE) to afford benzyl (3S)-3-[(3-benzyloxycyclobutyl)-tert-butoxycarbonyl-amino]pyrrolidine-1-carboxylate (B2, 8.9 g, 18.5 mmol, 82% yield) as a solid. LCMS (ES, m/z): 481 [M+H]⁺.

### Synthesis of B3

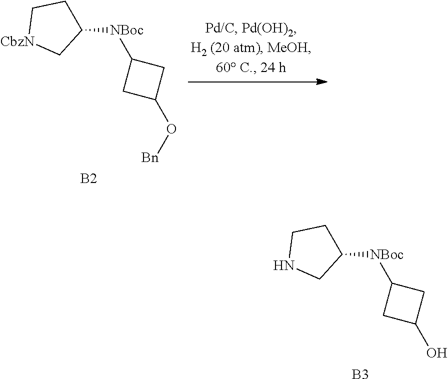 [SMILES: CC(C)(C)OC(=O)N(C1CC(OCc2ccccc2)C1)[C@H]1CCN(C(=O)OCc2ccccc2)C1 ; "B2" ; CC(C)(C)OC(=O)N(C1CC(O)C1)[C@H]1CCNC1 ; "B3"]

To a solution of benzyl (3S)-3-[(3-benzyloxycyclobutyl)-tert-butoxycarbonyl-amino]pyrrolidine-1-carboxylate (B2, 4 g, 8.32 mmol) in MeOH (300 mL) was added Pd/C (10% on Carbon, wetted with ca. 55% water) (443 mg, 4.16 mmol) and Pd(OH)₂ (20% on Carbon, wetted with ca. 55% water) (468 mg, 3.33 mmol) under N₂. The mixture was stirred for 60 h at 60° C. under H₂. The mixture was filtered through a Celite pad and concentrated under reduced pressure to afford tert-butyl N-(3-hydroxycyclobutyl)-N-[(3S)-pyrrolidin-3-yl]carbamate (B3, 1.5 g, 5.85 mmol, 70% yield). LCMS (ES, m/z): 257 [M+H]⁺.

### Synthesis of B4

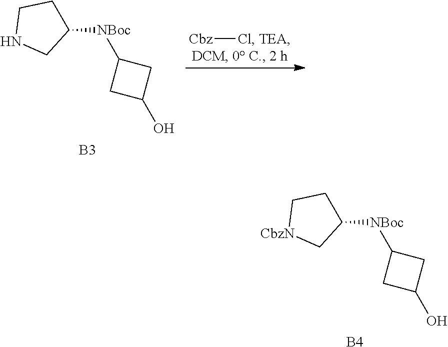

To a solution of tert-butyl N-(3-hydroxycyclobutyl)-N-[(3S)-pyrrolidin-3-yl]carbamate (B3, 1.5 g, 5.85 mmol) in DCM (15 mL) were added TEA (2.37 g, 23.4 mmol, 3.3 mL) and CbzCl (1.1 g, 6.44 mmol, 907.5 μL) at 0° C. The mixture was stirred for 2 h at 0° C. The resulting mixture was diluted with water (100 mL) and extracted with DCM (2×100 mL). The combined organic layers were washed with brine (150 mL), dried over anhydrous Na₂SO₄, and filtered. The filtrate was concentrated under reduced pressure and purified by silica gel column chromatography (50% EtOAc in PE) to afford benzyl (3S)-3-[tert-butoxycarbonyl-(3-hydroxycyclobutyl)amino]pyrrolidine-1-carboxylate (B4, 850 mg, 2.18 mmol, 37% yield) as a solid. ¹H NMR (400 MHz, DMSO-d₆) δ 7.44-7.24 (m, 5H), 5.07 (d, J=2.3 Hz, 2H), 4.96 (dd, J=23.5, 5.5 Hz, 1H), 4.47-4.11 (m, 2H), 3.89-3.68 (m, 1H), 3.54 (dd, J=19.2, 9.4 Hz, 1H), 3.48-3.36 (m, 1H), 3.32-3.14 (m, 1H), 2.47-2.34 (m, 2H), 2.31-2.00 (m, 2H), 2.00-1.90 (m, 1H), 1.39 (t, J=5.2 Hz, 9H).

### Synthesis of B5

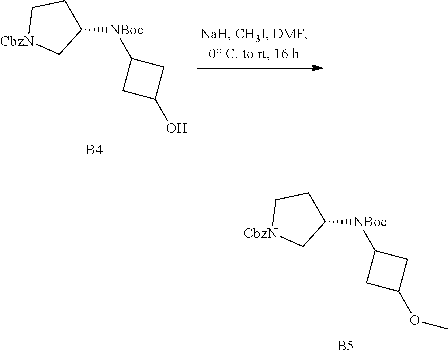 [SMILES: CC(C)(C)OC(=O)N(C1CC(O)C1)[C@H]1CCN(C(=O)OCc2ccccc2)C1 ; "B4" ; COC1CC(N(C(=O)OC(C)(C)C)[C@H]2CCN(C(=O)OCc3ccccc3)C2)C1 ; "B5"]

To a stirred solution of benzyl (3S)-3-[tert-butoxycarbonyl-(3-hydroxycyclobutyl)amino]pyrrolidine-1-carboxylate (B4, 0.65 g, 1.66 mmol) in DMF (8 mL) were added CH₃I (709 mg, 4.99 mmol) and NaH (120 mg, 4.99 mmol) at 0° C., and the reaction was stirred for 16 h at room temperature. The resulting mixture was diluted with water (30 mL) and extracted with EtOAc (3×30 mL). The combined organic layers were washed with water (3×80 mL), dried over anhydrous Na₂SO₄, and filtered. The filtrate was concentrated under reduced pressure and purified by silica gel column chromatography (17% EtOAc in PE) to afford benzyl (3S)-3-[tert-butoxycarbonyl-(3-methoxycyclobutyl)amino]pyrrolidine-1-carboxylate (B5, 0.55 g, 1.36 mmol, 82% yield) as a solid. LCMS (ES, m/z): 405 [M+H]⁺.

### Synthesis of B6

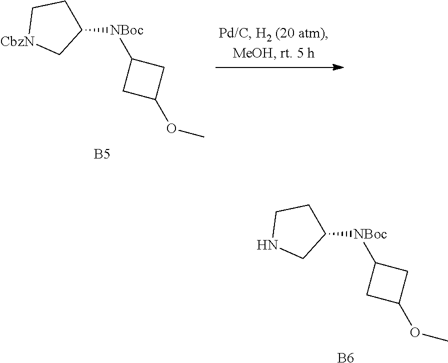 [SMILES: COC1CC(N(C(=O)OC(C)(C)C)[C@H]2CCN(C(=O)OCc3ccccc3)C2)C1 ; "B5" ; COC1CC(N(C(=O)OC(C)(C)C)[C@H]2CCNC2)C1 ; "B6"]

To a stirred solution of benzyl (3S)-3-[tert-butoxycarbonyl-(3-methoxycyclobutyl)amino]pyrrolidine-1-carboxylate (B5, 0.2 g, 494.4 μmol) in MeOH (5 mL) were added Pd/C (0.02 g, 187.9 μmol) at room temperature. The resulting mixture was stirred for 2 h at room temperature under hydrogen atmosphere. The resulting mixture was filtered, the filter cake was washed with MeOH (3×6 mL). The filtrate was concentrated under reduced pressure to afford tert-butyl N-(3-methoxycyclobutyl)-N-[(3S)-pyrrolidin-3-yl]carbamate (B6, 0.12 g, 443.8 μmol, 90% yield) as an oil. LCMS (ES, m/z): 270 [M+H]⁺.

### Synthesis of B7

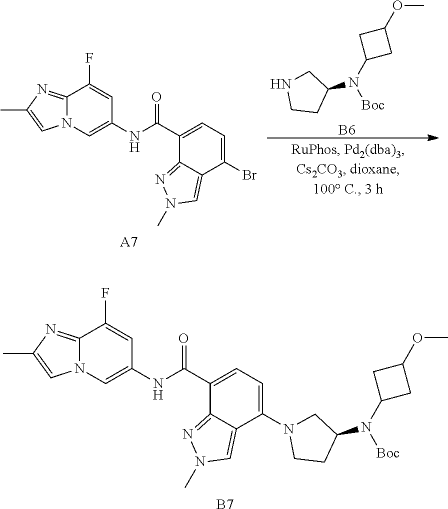

To a stirred solution of 4-bromo-N-(8-fluoro-2-methyl-imidazo[1,2-a]pyridin-6-yl)-2-methyl-indazole-7-carboxamide (A7, 0.13 g, 323.2 μmol) and tert-butyl N-(3-methoxycyclobutyl)-N-[(3S)-pyrrolidin-3-yl]carbamate (B6, 105 mg, 387.9 μmol) in dioxane (3 mL) were added RuPhos (30 mg, 64.6 μmol) Cs₂CO₃ (316 mg, 969.6 μmol) and RuPhos Pd G3 (270 mg, 323.2 μmol), and the reaction was stirred for 3 h at 100° C. under N₂. The resulting mixture was concentrated under reduced pressure and purified by silica gel column chromatography (89% EtOAc in PE) to afford tert-butyl N-[(3S)-1-[7-[(8-fluoro-2-methyl-imidazo[1,2-a]pyridin-6-yl)carbamoyl]-2-methyl-indazol-4-yl]pyrrolidin-3-yl]-N-(3-hydroxy-cyclobutyl)carbamate (B7, 0.13 g, 225 μmol, 70% yield) as a solid. LCMS (ES, m/z): 592 [M+H]⁺.

### Synthesis of Compound 268

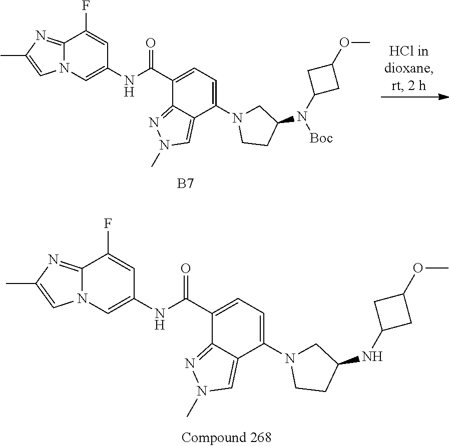

To a stirred solution of tert-butyl N-[(3S)-1-[7-[(8-fluoro-2-methyl-imidazo[1,2-a]-pyridin-6-yl)carbamoyl]-2-methyl-indazol-4-yl]pyrrolidin-3-yl]-N-(3-methoxycyclobutyl)-carbamate (B7, 0.1 g, 169 μmol) in dioxane (1 mL) were added HCl (4M in dioxane, 0.5 mL), and the reaction was stirred for 1 h at room temperature. The resulting mixture was concentrated under reduced pressure and purified by reversed-phase flash chromatography (Condition 3, Gradient 11) to afford N-(8-fluoro-2-methyl-imidazo[1,2-a]pyridin-6-yl)-4-[(3S)-3-[(3-methoxy-cyclobutyl)amino]pyrrolidin-1-yl]-2-methyl-indazole-7-carboxamide (Compound 268, 0.05 g, 101.7 μmol, 60% yield) as a solid. LCMS (ES, m/z): 492 [M+H]⁺. ¹H NMR (400 MHz, DMSO-d₆) δ 11.01 (s, 1H), 9.19 (d, J=1.6 Hz, 1H), 8.81 (d, J=6.1 Hz, 1H), 7.95-7.85 (m, 2H), 7.30 (dd, J=12.4, 1.7 Hz, 1H), 6.00 (dd, J=8.4, 3.5 Hz, 1H), 4.27 (s, 3H), 3.74 (s, 2H), 3.64-3.47 (m, 2H), 3.46-3.37 (m, 1H), 3.13 (d, J=4.5 Hz, 3H), 2.86 (p, J=7.9 Hz, 1H), 2.60-2.51 (m, 1H), 2.35 (s, 3H), 2.18-2.06 (m, 2H), 1.88 (dd, J=12.1, 6.2 Hz, 1H), 1.57 (dtd, J=11.0, 8.1, 2.9 Hz, 2H).

### Separation of Compounds 275 and 277

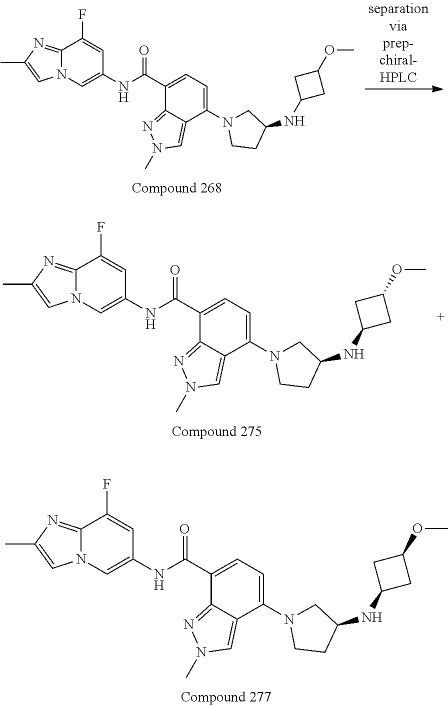

N-(8-fluoro-2-methyl-imidazo[1,2-a]pyridin-6-yl)-4-[(3S)-3-[(3-methoxycyclobutyl)-amino]pyrrolidin-1-yl]-2-methyl-indazole-7-carboxamide (Compound 268, 50 mg, 101.7 μmol) was separated by chiral-HPLC (Condition 5, Gradient 1) to afford N-(8-fluoro-2-methylimidazo[1,2-a]pyridin-6-yl)-4-((S)-3-(((1r,3S)-3-methoxycyclobutyl)amino)pyrrolidin-1-yl)-2-methyl-2H-indazole-7-carboxamide (Compound 275, 10 mg, 20.3 μmol, 20% yield) as a solid, and N-(8-fluoro-2-methylimidazo[1,2-a]pyridin-6-yl)-4-((S)-3-(((1s,3R)-3-methoxy-cyclobutyl)amino)pyrrolidin-1-yl)-2-methyl-2H-indazole-7-carboxamide (Compound 277, 25 mg, 50.9 μmol, 50% yield) as a solid.

Compound 275: LCMS (ES, m/z): 492 [M+H]⁺. ¹H NMR (400 MHz, DMSO-d₆) δ 11.01 (s, 1H), 9.19 (d, J=1.6 Hz, 1H), 8.82 (s, 1H), 7.95-7.85 (m, 2H), 7.30 (d, J=12.3 Hz, 1H), 6.01 (d, J=8.4 Hz, 1H), 4.27 (s, 3H), 3.94 (dq, J=6.8, 3.6, 3.0 Hz, 1H), 3.76-3.74 (m, 2H), 3.62 (d, J=8.4 Hz, 1H), 3.44-3.43 (m, 3H), 3.13 (s, 3H), 2.35 (s, 3H), 2.17-2.07 (m, 3H), 2.04-1.81 (m, 3H).

Compound 277: LCMS (ES, m/z): 492 [M+H]⁺. ¹H NMR (400 MHz, DMSO-d₆) δ 11.01 (s, 1H), 9.19 (d, J=1.6 Hz, 1H), 8.80 (s, 1H), 7.96-7.86 (m, 2H), 7.30 (dd, J=12.4, 1.7 Hz, 1H), 6.00 (d, J=8.4 Hz, 1H), 4.27 (s, 3H), 3.75 (d, J=11.8 Hz, 2H), 3.67-3.49 (m, 2H), 3.47-3.34 (m, 2H), 3.12 (s, 3H), 2.86 (q, J=7.8 Hz, 1H), 2.55 (dd, J=9.0, 5.0 Hz, 1H), 2.35 (s, 3H), 2.12 (tt, J=10.8, 5.4 Hz, 1H), 1.88 (dd, J=12.2, 6.3 Hz, 1H), 1.58 (q, J=8.7 Hz, 2H).

### Example 52: Synthesis of Compound 206

### Synthesis of B8

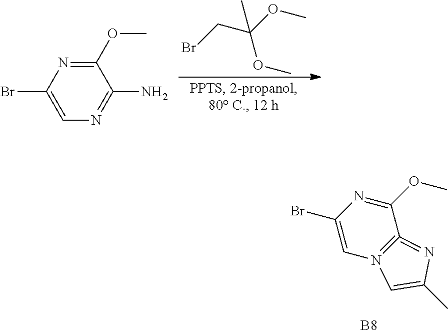

A mixture of 1-bromo-2,2-dimethoxy-propane (986 mg, 5.39 mmol, 1.1 eq), 5-bromo-3-methoxy-pyrazin-2-amine (1 g, 4.9 mmol, 1 eq) and 4-methylbenzenesulfonic acid pyridine (123 mg, 490 μmol, 0.1 eq) in 2-propanol (12.5 mL). The reaction mixture was stirred at 80° C. for 12 h. The reaction mixture was diluted with NaHCO₃ (100 mL), extracted with EtOAc (3×100 mL), dried over Na₂SO₄, and filtered. The filtrate was concentrated under reduced pressure and triturated with EtOAc (10 mL), and filtered. The filtrate was concentrated under reduced pressure and purified by silica gel column chromatography (50-100% EtOAc in PE) to afford 6-bromo-8-methoxy-2-methyl-imidazo[1,2-a]pyrazine (B8, 200 mg, 17% yield) as a solid. LCMS (ESI, m/z): 242.4 [M+H]⁺. ¹H NMR (400 MHz, Methanol-d₄) δ ppm 8.59 (s, 1H), 8.02 (s, 1H), 4.27 (s, 3H), 2.57 (d, J=0.75 Hz, 3H).

### Synthesis of B9

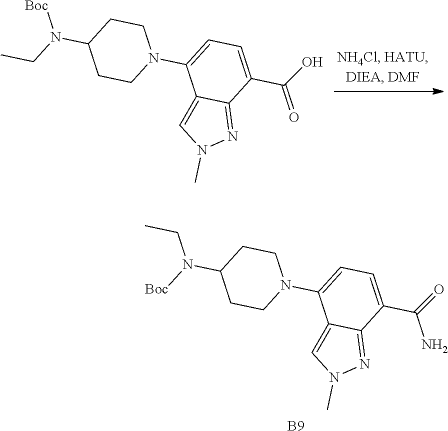 [SMILES: CCN(C(=O)OC(C)(C)C)C1CCN(c2ccc(C(=O)O)c3nn(C)cc23)CC1 ; CCN(C(=O)OC(C)(C)C)C1CCN(c2ccc(C(N)=O)c3nn(C)cc23)CC1 ; "B9"]

To a mixture of 4-[4-[tert-butoxycarbonyl (ethyl)amino]-1-piperidyl]-2-methyl-indazole-7-carboxylic acid (200 mg, 496 μmol, 1 eq) and NH₄Cl (106 mg, 1.99 mmol, 69.4 μL, 4 eq) in DCM (3 mL) was added o-(7-azabenzotriazol-1-yl)-N,N,N′,N′-tetramethyluronium hexafluorophosphate (226 mg, 596 μmol, 1.2 eq) and N,N-diisopropylethylamine (321 mg, 2.48 mmol, 432 μL, 5 eq). The reaction mixture was stirred at 25° C. for 12 h. The reaction mixture was diluted with H₂O (20 mL), extracted with DCM (3×20 mL), dried over Na₂SO₄, and filtered. The filtrate was concentrated under reduced pressure and purified by silica gel column chromatography (50-100% EtOAc in PE) to afford tert-butyl N-[1-(7-carbamoyl-2-methyl-indazol-4-yl)-4-piperidyl]-N-ethyl-carbamate (B9, 200 mg, 85% yield) as a solid. LCMS (ESI, m/z): 402.4 [M+H]⁺. ¹H NMR (400 MHz, DMSO-d₆) δ ppm 8.66 (s, 1H), 8.40 (br s, 1H), 8.08-8.25 (m, 1H), 7.84 (d, J=8.00 Hz, 1H), 7.45 (br s, 1H), 6.43 (d, J=8.00 Hz, 1H), 4.19 (s, 3H), 3.92 (br d, J=12.38 Hz, 2H), 3.62 (dq, J=10.62, 6.51 Hz, 2H), 2.90 (br t, J=11.76 Hz, 2H), 1.92 (br d, J=9.63 Hz, 2H), 1.71 (br d, J=10.38 Hz, 2H), 1.41 (s, 9H), 1.17 (t, J=7.07 Hz, 3H).

### Synthesis of B10

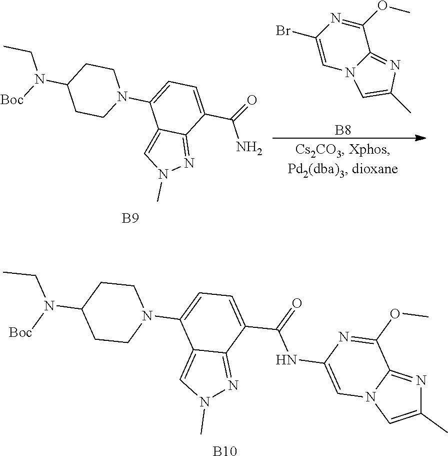

To a suspension of 6-bromo-8-methoxy-2-methyl-imidazo[1,2-a]pyrazine (A16-2, 65.8 mg, 271 μmol, 1.2 eq), tert-butyl N-[1-(7-carbamoyl-2-methyl-indazol-4-yl)-4-piperidyl]-N-ethyl-carbamate (B9, 91 mg, 226 μmol, 1 eq), Cs₂CO₃ (221 mg, 679 μmol, 3 eq), XPhos (21.6 mg, 45.3 μmol, 0.2 eq) and Pd₂(dba)₃ (20.7 mg, 22.6 μmol, 0.1 eq) in dioxane (1 mL). The reaction mixture was stirred at 80° C. for 2 h. The reaction mixture was diluted with H₂O (10 mL), extracted with EtOAc (3×10 mL), dried over Na₂SO₄, and filtered. The filtrate was concentrated under reduced pressure and purified by prep-HPLC (Condition 9, Gradient 1) to afford tert-butyl N-ethyl-N-[1-[7-[(8-methoxy-2-methyl-imidazo[1,2-a]pyrazin-6-yl)carbamoyl]-2-methyl-indazol-4-yl]-4-piperidyl]carbamate (B10, 74 mg, 55% yield) as a solid. LCMS (ESI, m/z): 563.3 [M+H]⁺. ¹H NMR (400 MHz, Methanol-d₄) δ ppm 9.26 (s, 1H), 8.56 (s, 1H), 8.08-8.18 (m, 2H), 6.57 (d, J=8.13 Hz, 1H), 4.35 (s, 3H), 4.31 (s, 3H), 4.13 (br d, J=13.38 Hz, 3H), 3.16-3.27 (m, 2H), 3.08 (br t, J=12.32 Hz, 2H), 2.57 (s, 3H), 1.97-2.11 (m, 2H), 1.85 (br d, J=11.01 Hz, 2H), 1.48 (s, 9H), 1.16 (t, J=6.88 Hz, 3H).

### Synthesis of Compound 206

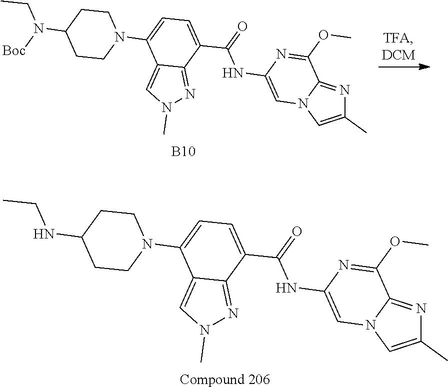

A mixture of tert-butyl N-ethyl-N-[1-[7-[(8-methoxy-2-methyl-imidazo[1,2-a]pyrazin-6-yl)carbamoyl]-2-methyl-indazol-4-yl]-4-piperidyl]carbamate (B10, 80 mg, 142 μmol) and trifluoroacetic acid (1.2 g, 10.5 mmol, 805 μL) in DCM (10 mL). The reaction mixture was stirred at 20° C. for 12 h. The reaction was concentrated under reduced pressure and purified by prep-HPLC (Condition 9, Gradient 2) to afford 4-[4-(ethylamino)-1-piperidyl]-N-(8-methoxy-2-methyl-imidazo[1,2-a]pyrazin-6-yl)-2-methyl-indazole-7-carboxamide (Compound 206, 17.5 mg, 25% yield) as a solid. LCMS (ESI, m/z): 463.3 [M+H]⁺. ¹H NMR (400 MHz, deuterium oxide) 0 ppm 8.43 (br d, J=2.6 Hz, 1H), 8.28 (s, 1H), 7.56 (br s, 1H), 7.29 (br s, 1H), 6.30 (br d, J=6.6 Hz, 1H), 4.06 (br s, 3H), 4.02-3.88 (m, 5H), 3.45 (br t, J=11.5 Hz, 1H), 3.18 (q, J=7.3 Hz, 2H), 3.05 (br t, J=12.3 Hz, 2H), 2.26 (br d, J=10.4 Hz, 2H), 2.16 (br s, 3H), 1.82-1.69 (m, 2H), 1.31 (t, J=7.3 Hz, 3H).

An analogous method was followed to obtain the following compounds.

| | Starting | |
|---|---|---|
| Compound | Material | Characterization |
| 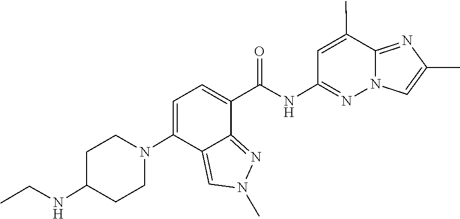 [SMILES: CCNC1CCN(c2ccc(C(=O)Nc3cc(C)c4nc(C)cn4n3)c3nn(C)cc23)CC1] Compound 186 | 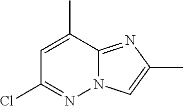 | Modification: The last step was performed using HCl/EtOAc. The solid dried in vacuum to give N-(2,8-dimethylimidazo [1,2-b]pyridazin-6-yl)-4- [4-(ethylamino)-1-piperidyl]-2- methyl-indazole-7-carboxamide (Compound 186, 28.6 mg, 47% yield) as a solid. LCMS (ESI, m/z): 447.3 [M + H]⁺. 1H NMR (400 MHz, Methanol-d₄) δ 8.81 (d, J = 1.0 Hz, 1H), 8.64 (s, 1H), 8.21-8.17 (m, 1H), 8.13 (s, 1H), 6.63 (d, J = 8.3 Hz, 1H), 4.34 (s, 3H), 4.18 (br d, J = 13.5 Hz, 2H), 3.50-3.37 (m, |
| | | 1H), 3.23-3.09 (m, 4H), 2.74 |
| | | (d, J = 1.0 Hz, 3H), 2.62 (d, J = 0.9 |
| | | Hz, 3H), 2.33-2.22 (m, 2H), 1.87 |
| | | (dq, J = 3.9, 12.2 Hz, 2H), |
| | | 1.37 (t, J = 7.3 Hz, 3H). |

An analogous method was followed to obtain the following compounds, using the following intermediate:

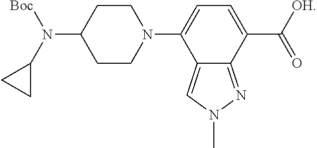 [SMILES: Cn1cc2c(N3CCC(N(C(=O)OC(C)(C)C)C4CC4)CC3)ccc(C(=O)O)c2n1]

| | Starting | |
|---|---|---|
| Compound | Material | Characterization |
| 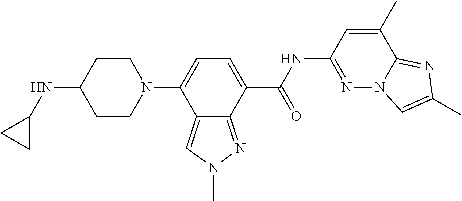 [SMILES: Cc1cn2nc(NC(=O)c3ccc(N4CCC(NC5CC5)CC4)c4cn(C)nc34)cc(C)c2n1] Compound 187 | 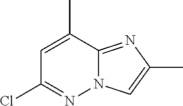 | The reaction mixture was filtered and the filter cake was purified by prep-HPLC (Condition 9, Gradient 3) to afford 4-[4- (cyclopropylamino)-1-piperidyl]- N-(2,8-dimethylimidazo- [1,2-b]pyridazin-6-yl)-2-methyl- indazole-7-carboxamide (Compound 187, 13 mg, 40% yield) as a solid. LCMS (ESI, m/z): 459.3 [M + H]'. ¹H NMR (400 MHz, D₂O) δ ppm 8.48 (br s, 1H), 8.24 (br s, 1H), 7.96 (br d, J = 8.13 Hz, 1H), 7.72 (s, 1H), 6.57 (br d, J = 8.50 |
| | | Hz, 1H), 4.28 (s, 3H), 4.06-4.15 |
| | | (m, 2H), 3.63 (br s, 1H), 3.17 |
| | | (br t, J = 12.57 Hz, 2H), 2.79 (br d, |
| | | J = 4.00 Hz, 1H), 2.48 (br d, J = |
| | | 12.51 Hz, 6H), 2.34 (br d, J = |
| | | 11.13 Hz, 2H), 1.76-1.89 (m, |
| | | 2H), 0.83-0.99 (m, 4H). |

### Example 53: Synthesis of B12

### Synthesis of B11

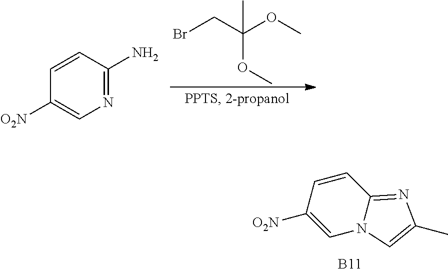

To a solution of 5-nitropyridin-2-amine (1 g, 7.19 mmol, 1 eq) and 1-bromo-2,2-dimethoxy-propane (1.45 g, 7.9 mmol, 1.1 eq) in 2-propanol (12.6 mL) was added 4-methylbenzenesulfonic acid pyridine (180 mg, 718 μmol, 0.1 eq), and the reaction was stirred at 80° C. for 12 h. The reaction mixture was diluted with saturated aqueous NaHCO₃ (30 mL), extracted with EtOAc (3×20 mL), washed with brine (2×40 mL), dried over Na₂SO₄, filtered and concentrated under reduced pressure to afford 2-methyl-6-nitro-imidazo[1,2-a]pyridine (B11, 1 g, 74% yield) as a solid. LCMS (ESI, m/z): 178.2 [M+H]⁺. ¹H NMR (400 MHz, DMSO-d₆) δ 9.80 (d, J=1.9 Hz, 1H), 7.95-7.84 (m, 2H), 7.58 (d, J=9.9 Hz, 1H), 2.38 (s, 3H).

### Synthesis of B12

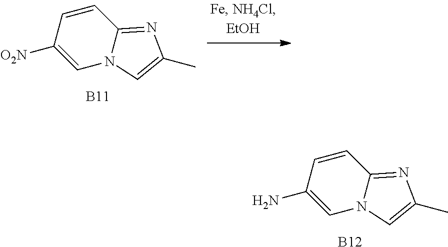

To a solution of 2-methyl-6-nitro-imidazo[1,2-a]pyridine (B11, 100 mg, 564 μmol, 1 eq) in EtOH (1.4 mL) was added Fe (94.5 mg, 1.69 mmol, 3 eq) and NH₄Cl (150 mg, 2.8 mmol, 5 eq). The reaction mixture was stirred at 80° C. for 16 h. The reaction mixture was filtered and the filtrate was concentrated under reduced pressure to afford 2-methyl imidazo[1,2-a]pyridin-6-amine (A10, 100 mg, 96% yield) as an oil. The material was used to the next step without any further purification. LCMS (ESI, m/z): 148.2 [M+H]⁺. ¹H NMR (400 MHz, Methanol-d₄) δ 7.78 (br s, 1H), 7.48-7.32 (m, 2H), 7.17 (br d, J=5.1 Hz, 1H), 2.39 (br s, 3H).

An analogous method was followed to obtain the following compounds.

| | Starting | |
|---|---|---|
| Compound | Material | Characterization |
| 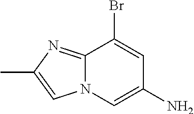 | 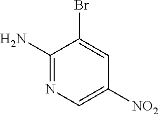 [SMILES: Nc1ncc([N+](=O)[O-])cc1Br] | Modifications: Step 1 was ran for 16 h. Step 2 was ran for 2 h. The residue was purified by silica gel column chromatography (9% MeOH in DCM) to afford 8- bromo-2-methyl-imidazo[1,2-a]pyridin-6-amine (C81, 350 mg, 1.55 mmol, 66% yield) as a solid. LCMS (ES, m/z): 226 [M + H]⁺. |
| C81 | | |

### Example 54: Synthesis of Compound 176

### Synthesis of B13

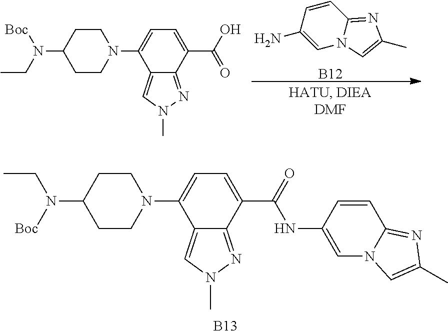

To a solution of 4-[4-[tert-butoxycarbonyl (ethyl)amino]-1-piperidyl]-2-methyl-indazole-7-carboxylic acid (50 mg, 124 μmol, 1 eq), o-(7-azabenzotriazol-1-yl)-N,N,N,N-tetramethyluronium hexafluorophosphate (56.6 mg, 149 μmol, 1.2 eq) and N-ethyl-N-isopropylpropan-2-amine (24 mg, 186 μmol, 32.4 μL, 1.5 eq) in N,N-dimethylformamide (0.8 mL) was added a solution of 2-methylimidazo[1,2-a]pyridin-6-amine (A10, 20.1 mg, 136 μmol, 1.1 eq) and N-ethyl-N-isopropylpropan-2-amine (24.0 mg, 186 μmol, 32.4 μL, 1.5 eq) in N,N-dimethylformamide (0.2 mL). The reaction mixture was stirred at 25° C. for 1 h. The reaction mixture was diluted with water (10 mL), extracted with EtOAc (3×10 mL), washed with brine (2×20 mL), dried over Na₂SO₄, and filtered. The filtrate was concentrated under reduced pressure and purified by prep-HPLC (Condition 10, Gradient 1) to afford tert-butyl N-ethyl-N-[1-[2-methyl-7-[(2-methylimidazo[1,2-a]pyridin-6-yl)carbamoyl]-indazol-4-yl]-4-piperidyl]-carbamate (B13, 20 mg, 15% yield) as a solid. LCMS (ESI, m/z): 532.4 [M+H]⁺.

### Synthesis of Compound 176

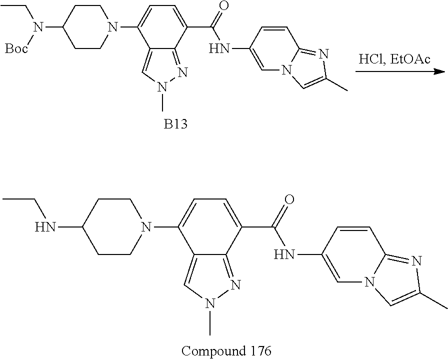

To a solution of tert-butyl N-ethyl-N-[1-[2-methyl-7-[(2-methylimidazo[1,2-a]pyridin-6-yl)carbamoyl]indazol-4-yl]-4-piperidyl]carbamate (B13, 18 mg, 33.8 μmol, 1 eq) in EtOAc (328 μL) was added HCl/EtOAc (4 M, 253 μL, 40 eq), and the reaction was stirred at 25° C. for 1 h. The reaction mixture was filtered and the filter cake was concentrated under reduced pressure to afford 4-[4-(ethylamino)-1-piperidyl]-2-methyl-N-(2-methylimidazo[1,2-a]pyridin-6-yl)-indazole-7-carboxamide (Compound 176, 8.26 mg, 57% yield) as a solid. LCMS (ESI, m/z): 432.4 [M+H]⁺. ¹H NMR (400 MHz, D₂O) δ 9.11 (s, 1H), 8.42-8.34 (m, 1H), 7.82 (d, J=8.1 Hz, 1H), 7.60 (dd, J=1.3, 9.8 Hz, 1H), 7.53-7.44 (m, 2H), 6.53-6.41 (m, 1H), 4.27-4.17 (m, 3H), 4.01 (br d, J=13.1 Hz, 2H), 3.51-3.36 (m, 1H), 3.16 (q, J=7.3 Hz, 2H), 3.07 (br t, J=12.3 Hz, 2H), 2.41-2.29 (m, 3H), 2.24 (br d, J=11.0 Hz, 2H), 1.84-1.69 (m, 2H), 1.29 (t, J=7.3 Hz, 3H).

An analogous method was followed to obtain the following compound.

| | Starting | |
|---|---|---|
| Compound | Material | Characterization |
| 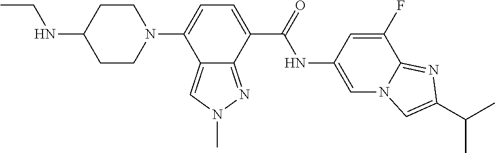 [SMILES: CCNC1CCN(c2ccc(C(=O)Nc3cc(F)c4nc(C(C)C)cn4c3)c3nn(C)cc23)CC1] Compound 177 | 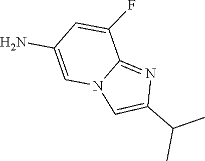 B16 | The reaction mixture was filtered and the filter cake was concentrated under reduced pressure to afford 4-[4- (ethylamino)-1-piperidyl]- N-(8-fluoro-2-isopropyl- imidazo[1,2- a]pyridin-6-yl)-2-methyl- indazole-7-carboxamide (Compound 177, 10 mg, 65% yield) as a solid. |
| | | LCMS (ESI, m/z): 478.4 |
| | | [M + H]⁺. ¹H NMR |
| | | (400 MHz, Deuterium |
| | | Oxide) δ 9.02 (d, J = 1.3 |
| | | Hz, 1H), 8.42 (s, 1H), |
| | | 7.77 (d, J = 8.1 Hz, 1H), |
| | | 7.65 (d, J = 1.1 Hz, 1H), |
| | | 7.55 (dd, J = 1.3, 10.9 Hz, |
| | | 1H), 6.46 (d, J = 8.4 Hz, |
| | | 1H), 4.26 (s, 3H), 4.03 |
| | | (br d, J = 13.1 Hz, 2H), |
| | | 3.51-3.36 (m, 1H), 3.16 |
| | | (q, J = 7.3 Hz, 2H), 3.12- |
| | | 2.97 (m, 3H), 2.24 (br |
| | | d, J = 9.9 Hz, 2H), 1.76 |
| | | (dq, J = 3.6, 12.2 Hz, 2H), |
| | | 1.33-1.26 (m, 9H). |
| 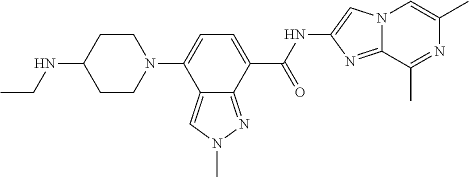 [SMILES: CCNC1CCN(c2ccc(C(=O)Nc3cn4cc(C)nc(C)c4n3)c3nn(C)cc23)CC1] Compound 181 | 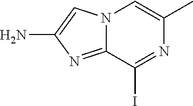 B21 | The reaction mixture was filtered and the filter cake was purified by prep- HPLC (Condition 9, Gradient 4) to afford N- (6,8-dimethylimidazo [1,2-a]pyrazin-2-yl)-4- [4-(ethylamino)-1- piperidyl]-2-methyl- indazole-7-carboxamide (Compound 181, 23.7 mg, 58% yield) as a solid. LCMS (ESI, m/z): 447.4 |
| | | [M + H]⁺. ¹H NMR (400 |
| | | MHz, D₂O) δ ppm 8.40 (s, |
| | | 1H), 8.14 (s, 1H), 7.73 (s, |
| | | 1H), 7.48 (d, J = 8.13 |
| | | Hz, 1H), 6.37 (d, J = 8.38 |
| | | Hz, 1H), 4.18 (s, 3H), |
| | | 4.06 (br d, J = 12.51 Hz, |
| | | 2H), 3.40-3.51 (m, 1H), |
| | | 3.18 (q, J = 7.42 Hz, 2H), |
| | | 3.06-3.14 (m, 2H), 2.26 |
| | | (br d, J = 10.26 Hz, 2H), |
| | | 2.17 (s, 3H), 2.02 (s, 3H), |
| | | 1.71-1.85 (m, 2H), 1.31 |
| | | (t, J = 7.25 Hz, 3H). |
| 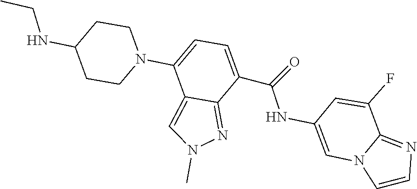 Compound 184 | 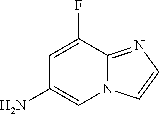 [SMILES: Nc1cc(F)c2nccn2c1] B16a | The reaction mixture was filtered and the filter cake was purified by prep-HPLC (Condition 9, Gradient 4) to afford 4-[4-(ethylamino)- 1-piperidyl]-N-(8- fluoroimidazo[1,2-a] pyridin-6-yl)-2-methyl- indazole-7-carboxamide (Compound 184, 30 mg, 68% yield) as a solid. LCMS (ESI, m/z): 464.3 [M + H]⁺. ¹H NMR (400 MHz, D₂O) δ ppm 8.90 |
| | | (d, J = 1.13 Hz, 1H), 8.26 |
| | | (s, 1H), 7.80 (t, J = 2.06 |
| | | Hz, 1H), 7.74 (d, J = 1.88 |
| | | Hz, 1H), 7.62-7.70 |
| | | (m, 1H), 7.40 (dd, |
| | | J = 10.94, 1.19 Hz, 1H), |
| | | 6.30 (d, J = 8.38 Hz, |
| | | 1H), 4.19 (s, 3H), 3.92 |
| | | (br d, J = 13.13 Hz, 2H), |
| | | 3.37-3.50 (m, 1H), |
| | | 3.17 (q, J = 7.25 Hz, |
| | | 2H), 3.01 (br t, J = 12.13 |
| | | Hz, 2H), 2.23 (br d, |
| | | J = 10.51 Hz, 2H), |
| | | 1.73 (qd, J = 12.15, |
| | | 3.56 Hz, 2H), 1.30 |
| | | (t, J = 7.32 Hz, 3H). |
| 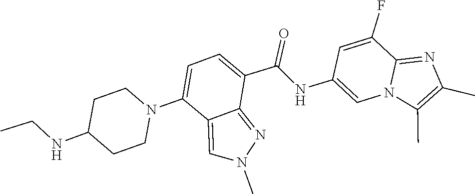 [SMILES: CCNC1CCN(c2ccc(C(=O)Nc3cc(F)c4nc(C)c(C)n4c3)c3nn(C)cc23)CC1] Compound 188 | 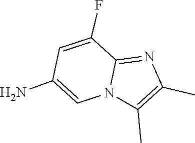 B16-i | The reaction mixture was concentrated under reduced pressure to afford 4-[4-(ethylamino)- 1-piperidyl]-N-(8- fluoro-2,3-dimethyl- imidazo[1,2-a]pyridin- 6-yl)-2-methyl-indazole- 7-carboxamide (Compound 188, 14 mg, 55% yield) as a solid. LCMS (ESI, m/z): 464.3 [M + H]⁺. ¹H NMR (400 MHz, Methanol-d4) |
| | | δ 9.33 (d, J = 1.1 Hz, |
| | | 1H), 8.61 (s, 1H), |
| | | 8.15 (d, J = 8.1 Hz, |
| | | 1H), 8.06 (dd, J = 1.4, |
| | | 11.3 Hz, 1H), 6.62 (d, |
| | | J = 8.1 Hz, 1H), 4.36 |
| | | (s, 3H), 4.15 (br d, J = |
| | | 13.3 Hz, 2H), 3.42 (tt, |
| | | J = 4.1, 11.6 Hz, 1H), |
| | | 3.21-3.07 (m, 4H), 2.57 |
| | | (d, J = 18.4 Hz, 6H), |
| | | 2.28 (br dd, J = 1.8, |
| | | 11.9 Hz, 2H), 1.86 |
| | | (dq, J = 3.9, 12.1 Hz, |
| | | 2H), 1.37 (t, J = 7.3 |
| | | Hz, 3H). |
| 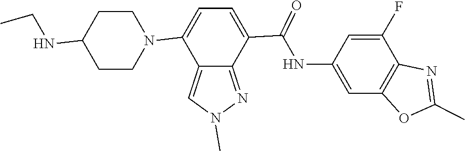 [SMILES: CCNC1CCN(c2ccc(C(=O)Nc3cc(F)c4nc(C)oc4c3)c3nn(C)cc23)CC1] Compound 200 | 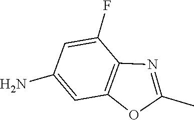 C25 | The filter cake was dried under vacuum to get 4-(4- (ethylamino)piperidin- 1-yl)-N-(4-fluoro-2- methylbenzo[d]- oxazol-6-yl)-2- methyl-2H-indazole- 7-carboxamide (Compound 200, 18.2 mg, 48% yield) as a solid. LCMS (ESI, m/z): 451.3 [M + H]⁺. |
| | | ¹H NMR (400 MHz, |
| | | D₂O) δ ppm 8.18 (s, |
| | | 1H), 7.68 (d, J = |
| | | 8.13 Hz, 1H), 7.39 |
| | | (s, 1H), 6.94 (br d, |
| | | J = 11.26 Hz, 1H), |
| | | 6.34-6.42 (m, 1H), |
| | | 4.10 (s, 3H), 3.92 |
| | | (br d, J = 12.88 |
| | | Hz, 2H), 3.36- |
| | | 3.46 (m, 1H), |
| | | 3.13-3.21 (m, 2H), |
| | | 2.98 (br t, J = 12.13 |
| | | Hz, 2H), 2.41 (s, |
| | | 3H), 2.18-2.29 (m, |
| | | 2H), 1.69-1.83 |
| | | (m, 2H), 1.30 (t, |
| | | J = 7.32 Hz, 3H). |
| 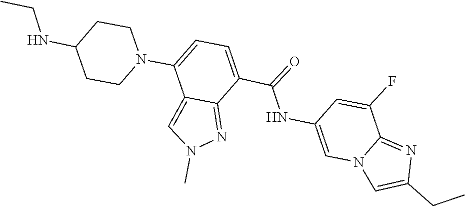 [SMILES: CCNC1CCN(c2ccc(C(=O)Nc3cc(F)c4nc(CC)cn4c3)c3nn(C)cc23)CC1] Compound 201 | 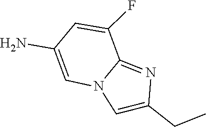 B16-ii | The solid was diluted with water and purified by freeze-dry to afford 4-[4- (ethylamino)-1- piperidyl]-N-(2- ethyl-8-fluoro- imidazo[1,2-a] pyridin-6-yl)-2- methyl-indazole- 7-carboxamide (Compound 201, 7.6 mg, 31% yield) as a solid. |
| | | LCMS (ESI, m/z): |
| | | 464.3 [M + H]⁺. |
| | | ¹H NMR (400 |
| | | MHz, D₂O) δ |
| | | 9.02 (s, 1H), 8.44 |
| | | (s, 1H), 7.80 (d, |
| | | J = 8.3 Hz, 1H), |
| | | 7.64-7.54 (m, 2H), |
| | | 6.49 (d, J = 8.3 |
| | | Hz, 1H), 4.26 (s, |
| | | 3H), 4.05 (br d, |
| | | J = 13.3 Hz, 2H), |
| | | 3.50-3.35 (m, 1H), |
| | | 3.23-3.02 (m, |
| | | 4H), 2.70 (q, J = |
| | | 7.5 Hz, 2H), 2.25 |
| | | (br d, J = 11.3 |
| | | Hz, 2H), 1.85- |
| | | 1.67 (m, 2H), 1.28 |
| | | (td, J = 7.4, 10.6 |
| | | Hz, 6H). |
| 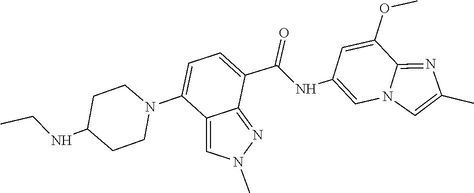 Compound 175 | 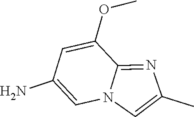 B16-iii | The reaction mixture was filtered and the filter cake was dried under vacuum to afford 4-(4-(ethylamino) piperidin-1-yl)-N- (8-methoxy-2-methyl- imidazo-[1,2-a] pyridin-6-yl)-2- methyl-2H-indazole- 7-carboxamide (Compound 175, 12 mg, 76% yield) as a solid. LCMS (ESI, m/z): |
| | | 462.3 [M + H]⁺. ¹H |
| | | NMR (400 MHz, |
| | | Deuterium Oxide) δ |
| | | ppm 8.74 (s, 1H), 8.35 |
| | | (s, 1H), 7.81 (br d, J = |
| | | 7.09 Hz, 1H), 7.49 |
| | | (br s, 1H), 6.90 (br d, |
| | | J = 3.42 Hz, 1H), 6.47 |
| | | (br d, J = 8.07 Hz, 1H), |
| | | 4.23 (s, 3H), 3.95- |
| | | 4.05 (m, 5H), 3.37- |
| | | 3.50 (m, 1H), 3.15 (q, |
| | | J = 7.13 Hz, 2H), 3.01- |
| | | 3.11 (m, 2H), 2.34 (s, |
| | | 3H), 2.19- 2.26 (m, |
| | | 2H), 1.70- 1.81 (m, |
| | | 2H), 1.27 (t, |
| | | J = 7.21 Hz, 3H). |
| 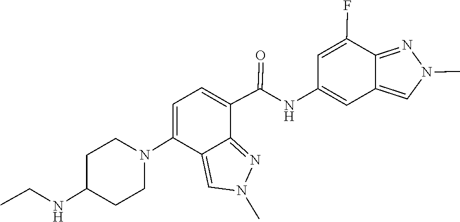 [SMILES: CCNC1CCN(c2ccc(C(=O)Nc3cc(F)c4nn(C)cc4c3)c3nn(C)cc23)CC1] Compound 182 | 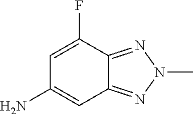 B16b | The reaction mixture was filtered and the filter cake was dried in vacuum to afford 4-(4- (ethylamino)piperidin- 1-yl)-N-(7-fluoro-2 -methyl-2H-indazol- 5-yl)-2-methyl-2H- indazole-7-carboxamide (Compound 182, 26 mg, 62% yield) as a solid. LCMS (ESI, m/z): 450.3 [M + H]⁺. ¹H NMR (400 MHz, D₂O) δ ppm 7.85 (s, 1H), 7.55 (d, J = 2.63 |
| | | Hz, 1H), 7.45 (t, J = 4.00 |
| | | Hz, 1H), 7.20 (d, |
| | | J = 1.25 Hz, 1H), |
| | | 6.50-6.60 (m, |
| | | 1H), 6.12 (d, |
| | | J = 8.13 Hz, 1H), |
| | | 3.84 (d, J = 10.51 |
| | | Hz, 6H), 3.68 (br |
| | | d, J = 12.38 Hz, |
| | | 2H), 3.18-3.30 |
| | | (m, 1H), 3.08 (q, |
| | | J = 7.25 Hz, 2H), |
| | | 2.66-2.78 (m, |
| | | 2H), 2.11 (br d, J = |
| | | 10.26 Hz, 2H), |
| | | 1.59-1.73 (m, |
| | | 2H), 1.27 (t, |
| | | J = 7.32 Hz, 3H). |

### Example 55: Synthesis of B16

### Synthesis of B14

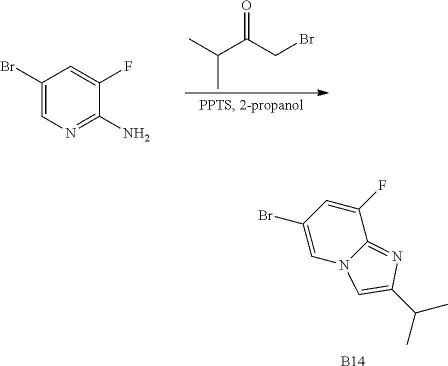

To a mixture of 5-bromo-3-fluoro-pyridin-2-amine (A37-1, 1 g, 5.24 mmol, 1 eq), 1-bromo-3-methyl-butan-2-one (950 mg, 5.76 mmol, 1.1 eq) in 2-propanol (12.6 mL) was added 4-methylbenzenesulfonic acid pyridine (131 mg, 523 μmol, 0.1 eq). The reaction mixture was stirred at 80° C. for 12 h. The reaction mixture was diluted with saturated aqueous NaHCO₃ (30 mL), extracted with EtOAc (3×20 mL), washed with brine (2×40 mL), dried over Na₂SO₄, filtered and concentrated under reduced pressure to afford 6-bromo-8-fluoro-2-isopropyl-imidazo[1,2-a]pyridine (B14, 900 mg, 61% yield) as an oil. LCMS (ESI, m/z): 259.0 [M+H]⁺.

### Synthesis of B15

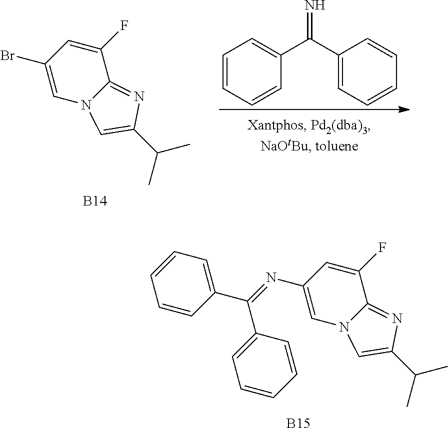

To a solution of 6-bromo-8-fluoro-2-isopropyl-imidazo[1,2-a]pyridine (B14, 225 mg, 875 μmol, 1 eq) and diphenylmethanimine (190 mg, 1.05 mmol, 1.2 eq) in toluene (5 mL) was added Pd₂(dba)₃ (80.1 mg, 87.5 μmol, 0.1 eq), NaO^{t}Bu (210 mg, 2.19 mmol, 205 μL, 2.50 eq) and XantPhos (50.6 mg, 87.5 μmol, 0.1 eq), and the reaction was stirred at 80° C. for 2 h. The reaction mixture was diluted with water (10 mL), extracted with EtOAc (3×10 mL), washed with brine (2×20 mL), dried over Na₂SO₄, and filtered. The filtrate was concentrated under reduced pressure and purified by silica gel column chromatography (0-17% EtOAc in PE) to afford N-(8-fluoro-2-isopropyl-imidazo[1,2-a]pyridin-6-yl)-1,1-diphenyl-methanimine (B15, 0.4 g, 19% yield) as an oil. LCMS (ESI, m/z): 358.2 [M+H]⁺. ¹H NMR (400 MHz, Methanol-d₄) δ 7.75-7.68 (m, 3H), 7.55-7.51 (m, 2H), 7.46 (d, J=7.9 Hz, 2H), 7.41-7.36 (m, 3H), 7.25-7.20 (m, 2H), 6.61 (dd, J=1.6, 11.8 Hz, 1H), 3.01 (quin, J=6.9 Hz, 1H), 1.31 (d, J=6.9 Hz, 6H).

### Synthesis of Int-B16

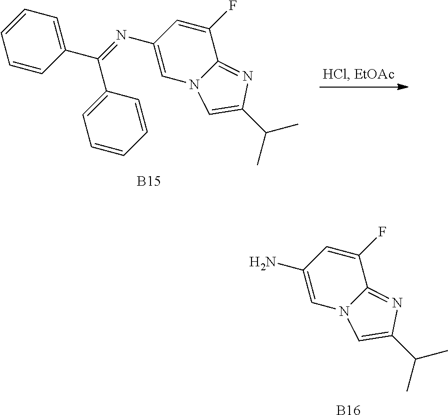

To a solution of N-(8-fluoro-2-isopropyl-imidazo[1,2-a]pyridin-6-yl)-1,1-diphenyl-methanimine (B15, 400 mg, 1.12 mmol, 1 eq) in EtOAc (8 mL) was added HCl/EtOAc (4 M, 8.4 mL). The reaction mixture was stirred at 25° C. for 1 h. The reaction mixture was concentrated under reduced pressure to get solid. The solid was triturated with MTBE for 4 h and filtered. The filter concentrated under reduced pressure to get 8-fluoro-2-isopropylimidazo[1,2-a]pyridin-6-amine (B16, 100 mg, 46% yield) as a solid. LCMS (ESI, m/z): 194.2 [M+H]⁺. ¹H NMR (400 MHz, Methanol-d₄) δ 7.85 (dd, J=0.6, 2.4 Hz, 1H), 7.77 (d, J=1.8 Hz, 1H), 7.32 (dd, J=1.7, 11.9 Hz, 1H), 3.23-3.11 (m, 1H), 1.41 (d, J=6.9 Hz, 6H).

An analogous method was followed to obtain the following intermediates.

| | Starting | |
|---|---|---|
| Compound | Material | Characterization |
| 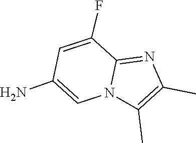 | 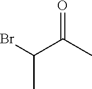 | The filter cake was dried in vacuum to get 8-fluoro-2,3- dimethyl-imidazo[1,2-a]pyridin-6-amine (B16-i, 0.16 g, 72% yield) as a solid. LCMS (ESI, m/z): 180.1 [M + H]⁻. ¹H NMR (400 MHz, Methanol-d₄) δ ppm 7.62 (d, J = 1.50 Hz, 1H), 7.33 (dd, J = 11.88, 1.63 Hz, 1H), 2.47 (s, 3H), 2.45 (s, 3H). |
| B16-i | | |
| 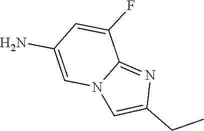 | 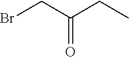 | The filter cake was dried in vacuum to get 2-ethyl-8- fluoroimidazo[1,2-a]pyridin-6-amine (B16-ii, 180 mg, 57% yield) as a solid. LCMS (ESI, m/z): 180.1 [M + H]⁻. |
| B16-ii | | |
| 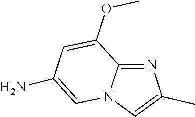 B16-iii | 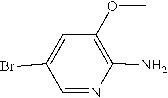 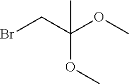 | The filter cake was dried in vacuum to afford 8- methoxy-2-methyl-imidazo[1,2-a]pyridin-6-amine (B16-iii, 220 mg, 94% yield) as a solid. LCMS (ESI, ppm 13.96-14.16 (m, 1H), 7.90 (d, J = 0.88 Hz, 1H), m/z): 178.3 [M + H]⁺. ¹H NMR (400 MHz, DMSO-d₆) δ 7.55 (d, J = 1.50 Hz, 1H), 6.91 (d, J = 1.50 Hz, 1H), 4.00 (s, 3H), 2.36 (d, J-0.75 Hz, 3H). |

An analogous method was followed to obtain the following intermediate, beginning on step 2.

| | Starting | |
|---|---|---|
| Compound | Material | Characterization |
| 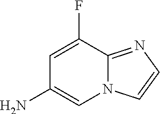 [SMILES: Nc1cc(F)c2nccn2c1] B16a | 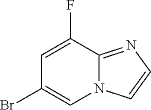 [SMILES: Fc1cc(Br)cn2ccnc12] | Modification: Starting from the synthesis of B15, using Cs₂CO₃ was used in place of NaOtBu. In the synthesis of B16, the reaction was ran in dioxane. The filter cake was dried in vacuum to get 8- fluoroimidazo[1,2-a]pyridin-6-amine (B16a, 700 mg, 79% yield) as a solid. LCMS (ESI, m/z): 152.3 [M + H]⁺. ¹H NMR (400 MHz, DMSO-d6) δ ppm 8.28 (s, 1H), 8.08 (d, J = 1.38 Hz, 1H), 7.87 (s, 1H), 7.40 (d, J = 12.38 Hz, 1H). |
| 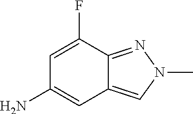 B-16b | 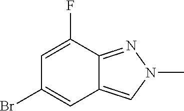 B27 | The mixture was filtered and the filter cake was dried in vacuum to afford 7-fluoro-2-methyl-indazol-5- amine (B16b, 600 mg, 98% yield) as a solid. LCMS (ESI, m/z): 166.2 [M + H]⁺. ¹H NMR (400 MHz, DMSO-d6) δ ppm 9.63-10.90 (m, 2H), 8.58 (d, J = 2.75 Hz, 1H), 7.62 (d, J = 1.63 Hz, 1H), 7.07 (dd, J = 11.76, 1.63 Hz, 1H), 4.22 (s, 3H). |
| 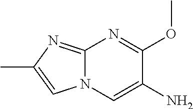 | 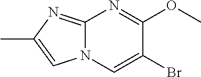 | Modifications: t-BuXPhos is used in place of XPhos in step 2, and the last step is ran in THF. The filtrate was concentrated under reduced pressure to afford 7-methoxy-2-methyl-imidazo[1,2- a]pyrimidin-6-amine (B16c, 78 mg, 437.7 umol, 99.9% yield) as an oil. LCMS (ES, m/z): 179 [M + H]⁺. |
| B16c | | |

### Example 56: Synthesis of Compound 178

### Synthesis of B17

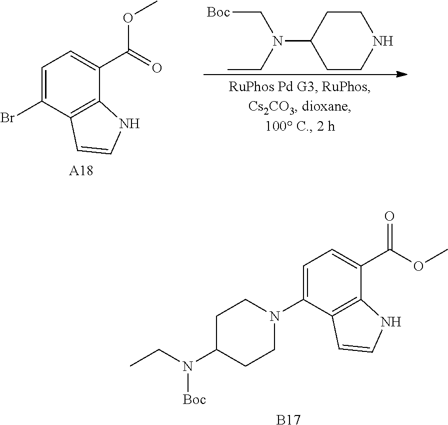

To a solution of methyl 4-bromo-1H-indole-7-carboxylate (A18, 200 mg, 787 μmol) and tert-butyl N-ethyl-N-(4-piperidyl)carbamate (359 mg, 1.57 mmol) in dioxane (4 mL) was added Cs₂CO₃ (769 mg, 2.4 mmol), Ruphos (73.4 mg, 157 μmol), and Ruphos Pd G3 (65.8 mg, 78.7 μmol) under N₂, and the reaction was stirred at 100° C. for 2 h. The resulting mixture was allowed to cool to room temperature, poured into water (20 mL), and extracted with DCM (3×10 mL). The combined organic phase was washed with brine (10 mL), dried with anhydrous Na₂SO₄, and filtered. The filtrate was concentrated under reduced pressure and purified by silica gel column chromatography (50% EtOAc in PE) to give methyl 4-[4-[tert-butoxycarbonyl(ethyl)amino]-1-piperidyl]-1H-indole-7-carboxylate (B17, 150 mg, 47% yield) as a solid. LCMS (ES, m/z): 403.4 [M+H]⁺. ¹H NMR (400 MHz, CDCl₃) δ ppm 9.95 (br s, 1H), 7.81 (d, J=8.25 Hz, 1H), 7.24 (br s, 1H), 6.58 (br s, 2H), 4.16-4.37 (m, 1H), 3.98 (br d, J=12.76 Hz, 2H), 3.95 (s, 3H), 3.13-3.29 (m, 2H), 2.83-3.11 (m, 2H), 1.88-2.05 (m, 2H), 1.86 (br d, J=9.63 Hz, 2H), 1.50 (s, 9H), 1.16 (t, J=7.00 Hz, 3H).

### Synthesis of B18

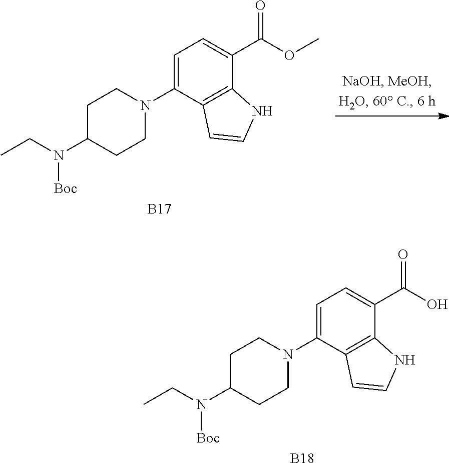

To a solution of methyl 4-[4-[tert-butoxycarbonyl(ethyl)amino]-1-piperidyl]-1H-indole-7-carboxylate (B17, 350 mg, 871 μmol) in MeOH (3.5 mL) and water (3.5 mL) was added NaOH (174 mg, 4.36 mmol), and the reaction was stirred at 60° C. for 6 h. One additional vial was set up as described above and two reaction mixtures were combined. The reaction mixture was concentrated under reduced pressure, diluted with water (20 mL), and extracted with EtOAc (3×20 mL). The aqueous phase was acidified to pH=4 with HCl aq. (1 M) and the resulting mixture was extracted with EtOAc (3×20 mL). The combined organic phase was washed with brine (20 mL), dried with anhydrous Na₂SO₄, filtered, and concentrated to afford 4-(4-((tert-butoxycarbonyl)(ethyl)amino)piperidin-1-yl)-1H-indole-7-carboxylic acid (B18, 410 mg, 71% yield) as a solid. LCMS (ES, m/z): 388.3 [M+H]⁺. ¹H NMR (400 MHz, CDCl₃) δ ppm 9.91 (br s, H), 7.91 (d, J=8.38 Hz, 1H), 7.24 (br t, J=2.69 Hz, 1H), 6.52-6.61 (m, 2H), 4.15-4.40 (m, 1H), 4.05 (br d, J=12.51 Hz, 2H), 3.21 (br s, 2H), 3.00 (br t, J=11.88 Hz, 2H), 1.96 (br d, J=5.88 Hz, 2H), 1.85-1.90 (m, 2H), 1.51 (s, 9H), 1.16 (t, J=6.94 Hz, 3H).

### Synthesis of B19

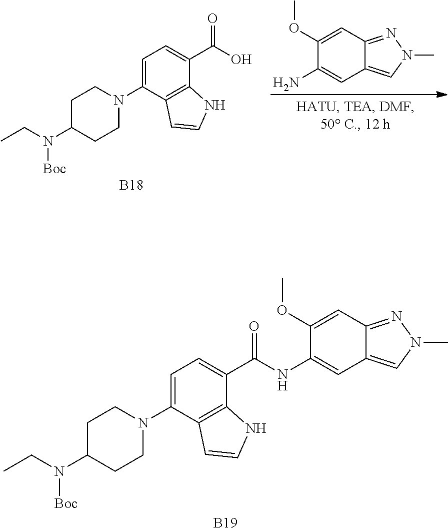

To a solution of 4-[4-[tert-butoxycarbonyl(ethyl)amino]-1-piperidyl]-1H-indole-7-carboxylic acid (B18, 400 mg, 1.03 mmol) in dimethyl formamide (20 mL) was added N,N-diethylethanamine (313 mg, 3.1 mmol, 431 μL) and o-(7-azabenzotriazol-1-yl)-n,n,n,n-tetramethyluroniumhexafluorophosphate (588 mg, 1.55 mmol) and 6-methoxy-2-methyl-indazol-5-amine (274 mg, 1.55 mmol) under N₂, and the reaction was stirred at 50° C. for 12 h. The reaction mixture was poured into water (80 mL) and extracted with DCM (3×80 mL). The combined organic phase was washed with brine (100 mL), dried with anhydrous Na₂SO₄, and filtered. The filtrate was concentrated under reduced pressure and purified by silica gel column chromatography (50% EtOAc in PE) to give tert-butyl N-ethyl-N-[1-[7-[(6-methoxy-2-methyl-indazol-5-yl) carbamoyl]-1H-indol-4-yl]-4-piperidyl]carbamate (B19, 300 mg, 53% yield) as a solid. LCMS (ES, m/z): 547.4 [M+H]⁺. ¹H NMR (400 MHz, CDCl₃) δ ppm 10.53 (br s, 1H), 8.86 (s, 1H), 8.76 (s, 1H), 8.03 (s, 1H), 7.80 (s, 1H), 7.50 (d, J=8.16 Hz, 1H), 7.05 (s, 1H), 6.56-6.66 (m, 2H), 4.17 (s, 3H), 4.03 (s, 3H), 3.92-3.99 (m, 2H), 3.23 (br d, J=1.51 Hz, 1H), 2.96 (s, 2H), 2.89 (s, 2H), 1.92-2.04 (m, 2H), 1.85-1.90 (m, 2H), 1.51 (s, 9H), 1.17 (t, J=6.90 Hz, 3H).

### Synthesis of Compound 178

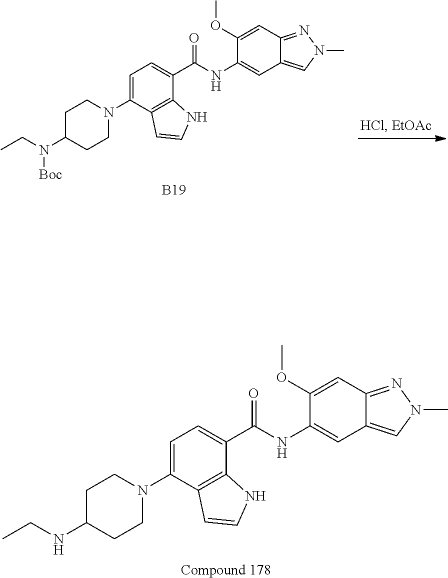

To a solution of tert-butyl N-ethyl-N-[1-[7-[(6-methoxy-2-methyl-indazol-5-yl)-carbamoyl]-1H-indol-4-yl]-4-piperidyl]carbamate (B19, 150 mg, 146 μmol) in EtOAc (2 mL) was added HCl/EtOAc (4 M, 2 mL), and the reaction was stirred at 25° C. for 2 h. The mixture was filtered and the filter cake was concentrated under reduced pressure to afford 4-[4-(ethylamino)-1-piperidyl]-N-(6-methoxy-2-methyl-indazol-5-yl)-1H-indole-7-carboxamide (Compound 178, 52 mg, 44%, as HCl salt) as a solid. LCMS (ES, m/z): 447.4 [M+H]. ¹H NMR (400 MHz, Methanol-d₄) δ ppm 8.82 (s, 1H), 8.75 (s, 1H), 7.86-7.88 (m, 1H), 7.61-7.64 (m, 1H), 7.53 (br d, J=8.13 Hz, 1H), 7.25 (s, 1H), 7.03 (d, J=3.38 Hz, 1H), 4.36 (s, 3H), 4.14 (s, 3H), 4.07 (br d, J=12.13 Hz, 2H), 3.94-4.01 (m, 2H), 3.75-3.82 (m, 1H), 3.24 (q, J=7.25 Hz, 2H), 2.44-2.55 (m, 4H), 1.42 (t, J=7.25 Hz, 3H).

An analogous method was followed to obtain the following compounds.

| | Starting | |
|---|---|---|
| Compound | Materials | Characterization |
| 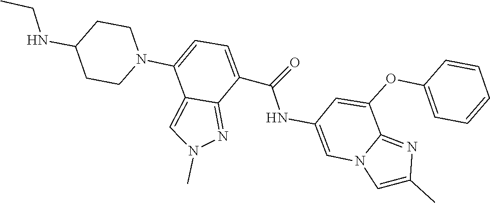 [SMILES: CCNC1CCN(c2ccc(C(=O)Nc3cc(Oc4ccccc4)c4nc(C)cn4c3)c3nn(C)cc23)CC1] Compound 183 | 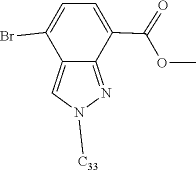 C33 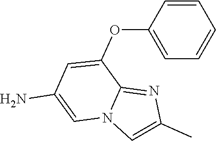 | The first step was ran for 12 h. The mixture was concentrated under reduced pressure to afford 4-(4-(ethylamino)- piperidin-1-yl)-2-methyl- N-(2-methyl-8- phenoxyimidazo- [1,2-a]pyridin-6-yl)-2H- indazole-7-carboxamide (Compound 183, 32 mg, 89% yield). LCMS (ESI, m/z): 524.5 [M + H]⁺. ¹H NMR (400 MHz, Methanol-d4) δ ppm 9.31 (d, J = 1.38 Hz, 1H), 8.58 (s, 1H), 8.05-8.11 (m, 2H), 7.56-7.63 (m, 2H), 7.35-7.44 (m, 3H), 7.33 (d, J = 1.38 Hz, 1H), 6.59 (d, J = 8.13 |
| | | Hz, 1H), 4.21 (s, 3H), |
| | | 4.11 (br d, J = 13.51 |
| | | Hz, 2H), 3.37-3.42 |
| | | (m, 1H), 3.05-3.21 |
| | | (m, 4H), 2.58 (s, 3H), |
| | | 2.26 (br dd, J = 11.94, |
| | | 1.69 Hz, 2H), 1.84 |
| | | (qd, J = 12.15, 3.69 |
| | | Hz, 2H), 1.36 (t, J = |
| | | 7.32 Hz, 3H). |
| 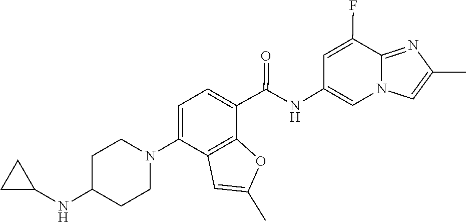 [SMILES: Cc1cn2cc(NC(=O)c3ccc(N4CCC(NC5CC5)CC4)c4cc(C)oc34)cc(F)c2n1] Compound 185 | 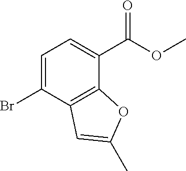 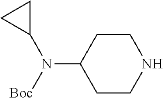 [SMILES: CC(C)(C)OC(=O)N(C1CCNCC1)C1CC1] Boc B24 | The mixture was filtered and the filter cake was dried to afford 4-[4- (cyclopropyl- amino)-1-piperidyl]- N-(8-fluoro-2- methyl-imidazo[1,2- a]pyridin-6-yl)-2- methyl-benzofuran- 7-carboxamide (Compound 185, 6.5 mg, 73%, as HCl salt) as a solid. LCMS (ES, m/z): 462.4 [M + H]⁺ ¹H NMR (400 MHz, Methanol-d₄) δ ppm 10.15 (s, 1H), 9.48 (s, 1H), 8.12 (s, 1H), 8.00 (d, J = 10.88 Hz, 1H), |
| | | 7.80 (d, J = 8.38 Hz, |
| | | 1H), 6.86 (d, J = 8.50 |
| | | Hz, 1H), 6.68 (s, 1H), |
| | | 3.93 (br d, J = 13.26 |
| | | Hz, 2H), 3.49-3.56 |
| | | (m, 1H), 2.96-3.06 |
| | | (m, 2H), 2.80-2.90 (m, |
| | | 1H), 2.58 (s, 6H), 2.30- |
| | | 2.40 (m, 2H), 1.83- |
| | | 1.95 (m, 2H), 0.97- |
| | | 1.03 (m, 2H), 0.89- |
| | | 0.94 (m, 2H). ¹⁹F |
| | | NMR (376 MHz, |
| | | Methanol-d₄) δ |
| | | ppm −134.255. |
| 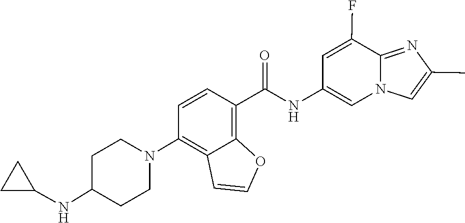 [SMILES: Cc1cn2cc(NC(=O)c3ccc(N4CCC(NC5CC5)CC4)c4ccoc34)cc(F)c2n1] Compound 191 | 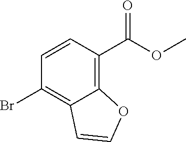 C27 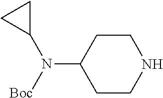 [SMILES: CC(C)(C)OC(=O)N(C1CCNCC1)C1CC1] | Step 2 was ran in THF as well. The mixture was filtered and the filter cake was dried to give 4-[4- (cyclopropylamino)-1- piperidyl]-N-(8-fluoro-2- methyl-imidazo[1,2-a] pyridin-6-yl) benzofuran- 7-carboxamide (Compound 191, 34.4 mg, 56%, as HCl salt) as a solid. LCMS (ES, m/z): 448.3 [M + H]⁺ ¹H NMR (400 MHz, Methanol-d4) δ ppm 9.49 (s, 1H), 8.11 (s, 1H), 8.01 (d, |
| | | J = 12.01 Hz, 1H), |
| | | 7.89-7.96 (m, 2H), |
| | | 7.09 (d, J = 2.13 Hz, |
| | | 1H), 6.92 (d, J = 8.38 |
| | | Hz, 1H), 3.94-4.03 |
| | | (m, 2H), 3.49-3.60 |
| | | (m, 1H), 3.07 (br t, |
| | | J = 11.94 Hz, 2H), |
| | | 2.80-2.94 (m, 1H), |
| | | 2.58 (s, 3H), 2.35 |
| | | (br d, J = 13.01 Hz, |
| | | 2H), 1.91 (qd, J = |
| | | 12.17, 4.13 Hz, 2H), |
| | | 0.97-1.04 (m, 2H), |
| | | 0.89-0.96 (m, 2H). |
| | | ¹⁹F NMR (376 MHz, |
| | | Methanol-d₄) δ ppm |
| | | −134.21 (s, 1F). |
| 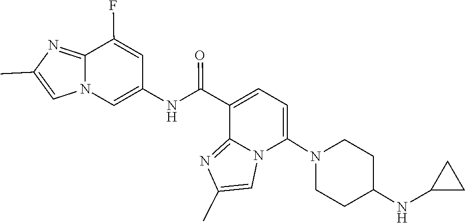 [SMILES: Cc1cn2cc(NC(=O)c3ccc(N4CCC(NC5CC5)CC4)n4cc(C)nc34)cc(F)c2n1] Compound 210 | 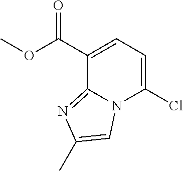 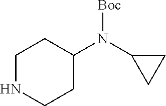 [SMILES: CC(C)(C)OC(=O)N(C1CCNCC1)C1CC1] | The reaction mixture was filtered and the filter cake was dried in vacuum to give 5-(4- (cyclopropylamino) piperidin- 1-yl)-N-(8-fluoro-2- methylimidazo[1,2- a]pyridin-6-yl)-2- methylimidazo[1,2-a]- pyridine-8-carboxamide (Compound 210, 19 mg, 83% yield) as a solid. LCMS (ESI, m/z): 462.4 [M + H]⁺. ¹H NMR (400 MHz, D₂O) δ ppm 9.07 (s, 1H), 8.38 (d, J = 8.25 Hz, 1H), 7.85 |
| | | (br s, 1H), 7.77 (br d, |
| | | J = 10.51 Hz, 1H), 7.65 |
| | | (s, 1H), 6.99 (d, J = 8.38 |
| | | Hz, 1H), 3.74 (br d, J = |
| | | 12.38 Hz, 2H), 3.56- |
| | | 3.67 (m, 1H), 3.04-3.14 |
| | | (m, 2H), 2.75-2.82 |
| | | (m, 1H), 2.51 (s, 3H), |
| | | 2.48 (s, 3H), 2.34-2.41 |
| | | (m, 2H), 1.88-2.00 (m, |
| | | 2H), 0.83-0.97 (m, 4H). |

### Example 57: Synthesis of Compound 190

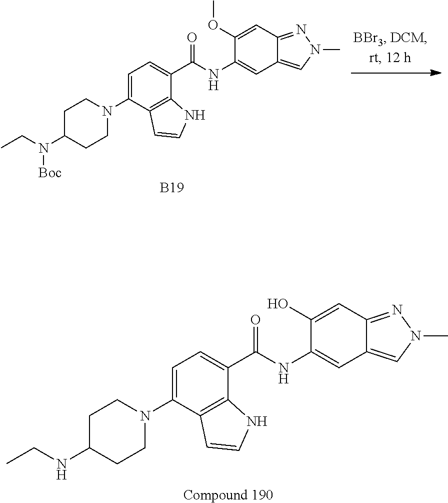

To a solution of tert-butyl N-ethyl-N-[1-[7-[(6-methoxy-2-methyl-indazol-5-yl)-carbamoyl]-1H-indol-4-yl]-4-piperidyl]carbamate (B19, 150 mg, 274 μmol) in DCM (3 mL) was added boron tribromide (1 M, 0.5 mL) at 0° C., and the reaction was stirred at 25° C. for 12 h. The reaction mixture was blow-dried with N₂. The crude product was purified by prep-HPLC (Condition 9, Gradient 2) to afford 4-[4-(ethylamino)-1-piperidyl]-N-(6-hydroxy-2-methyl-indazol-5-yl)-1H-indole-7-carboxamide (Compound 190, 4.16 mg, 3%, as HCl salt) as a solid. LCMS (ES, m/z): 433.4 [M+H]⁺. ¹H NMR (400 MHz, Methanol-d₄) δ ppm 8.86 (s, 1H), 8.68 (s, 1H), 7.71-7.80 (m, 1H), 7.51 (s, 1H), 7.15 (br d, J=8.13 Hz, 1H), 7.05 (s, 1H), 6.82 (d, J=3.25 Hz, 1H), 4.31 (s, 3H), 4.05 (br d, J=12.76 Hz, 2H), 3.56-3.59 (m, 1H), 3.45-3.55 (m, 2H), 3.21 (q, J=7.17 Hz, 2H), 2.42 (br d, J=13.01 Hz, 2H), 2.16-2.26 (m, 2H), 1.40 (t, J=7.25 Hz, 3H).

### Example 58: Synthesis of B21

### Synthesis of B20

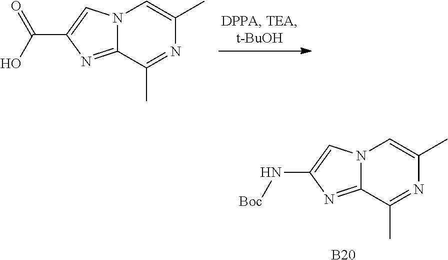

To a suspension of 6,8-dimethylimidazo[1,2-a]pyrazine-2-carboxylic acid (1 g, 5.23 mmol, 1 eq) in tert-butanol (10 mL) was added triethylamine (1.59 g, 15.6 mmol, 2.2 mL, 3 eq), and the reaction was stirred for 10 min at room temperature under N₂. Diphenyl phosphorazidate (1.44 g, 5.23 mmol, 1.13 mL, 1 eq) was added, and the reaction was stirred for an additional 12 h at 85° C. The resulting mixture was concentrated under reduced pressure, diluted with saturated aqueous bicarbonate (10 mL), extracted with EtOAc (3×10 mL), and washed with brine (2×10 mL). The resulting mixture was dried over Na₂SO₄, filtered, concentrated under reduced pressure, and purified by silica gel column chromatography (50-67% EtOAc in PE) to afford tert-butyl N-(6,8-dimethylimidazo[1,2-a]pyrazin-2-yl)carbamate (B20, 1.1 g, 38% yield) as a solid. LCMS (ESI, m/z): 263.1 [M+H]⁺. ¹H NMR (400 MHz, CDCl₃) δ ppm 7.80 (br s, 1H), 7.70 (s, 1H), 7.44 (br s, 1H), 2.80 (s, 3H), 2.48 (s, 3H), 1.54 (s, 9H).

### Synthesis of B21

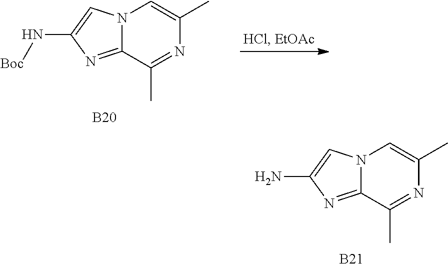

To a solution of tert-butyl N-(6,8-dimethylimidazo[1,2-a]pyrazin-2-yl)carbamate (B20, 550 mg, 2.1 mmol) in EtOAc (11 mL) was added HCl/EtOAc (4 M, 11 mL). The reaction mixture was stirred at 25° C. for 8 h. The reaction mixture was filtered and the filter cake was dried in vacuum to get 6,8-dimethylimidazo[1,2-a]pyrazin-2-amine (B21, 800 mg, 92% yield) as a solid. LCMS (ESI, m/z): 163.7 [M+H]⁺. ¹H NMR (400 MHz, DMSO-d₆) δ ppm 8.40 (s, 1H), 7.67 (s, 1H), 2.77 (s, 3H), 2.46 (s, 3H).

### Example 59: Synthesis of B24

### Synthesis of B22

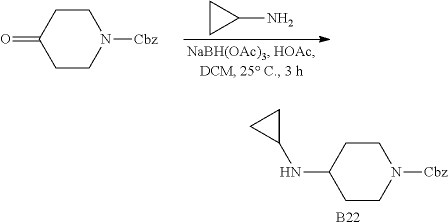

To a solution of benzyl 4-oxopiperidine-1-carboxylate (10 g, 42.9 mmol, 8.5 mL) and cyclopropanamine (2.7 g, 47 mmol, 3.3 mL) in DCM (250 mL) was added acetic acid (257 mg, 4.3 mmol) and NaBH(OAc)₃ (20 g, 94 mmol) under N₂, and the reaction was stirred at 25° C. for 3 h. Three additional vials were set up as described above and four reaction mixtures were combined. The mixture was poured into saturated NaHCO₃ (1 L) and the resulting mixture was extracted with DCM (3×400 mL). The combined organic phases were dried over Na₂SO₄, filtered and concentrated to afford benzyl 4-(cyclopropylamino)piperidine-1-carboxylate (B22, 45 g, 96% yield) as a solid. The crude product was used to next step directly. ¹H NMR (400 MHz, CDCl₃) δ ppm 7.29-7.40 (m, 5H), 5.13 (s, 2H), 4.12 (br s, 2H), 2.90 (br t, J=10.73 Hz, 2H), 2.78 (tt, J=10.34, 3.91 Hz, 1H), 2.12-2.17 (m, 1H), 2.10 (br s, 1H), 1.94 (br d, J=10.54 Hz, 2H), 1.23-1.40 (m, 2H), 0.42-0.54 (m, 2H), 0.30-0.39 (m, 2H).

### Synthesis of B23

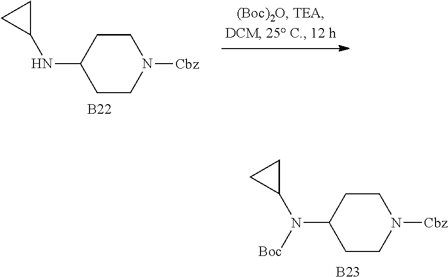

To a solution of benzyl 4-(cyclopropylamino)piperidine-1-carboxylate (B22, 5 g, 18 mmol) in DCM (50 mL) was added di-tert-butyldicarbonate (5.97 g, 27.3 mmol, 6.3 mL) and triethylamine (3.7 g, 36.5 mmol, 5.1 mL), and the reaction was stirred at 25° C. for 12 h. Eight additional vials were set up as described above and nine reaction mixtures were combined. The mixture was poured into H₂O (800 mL) and the resulting mixture was extracted with DCM (3×300 mL). The combined organic phases were dried over Na₂SO₄, and filtered. The filtrate was concentrated under reduced pressure and purified by silica gel column chromatography (50-100% EtOAc in PE) to afford benzyl 4-[tert-butoxycarbonyl(cyclopropyl)amino]piperidine-1-carboxylate (B23, 50 g, 81% yield) as an oil. ¹H NMR (400 MHz, CDCl₃) δ ppm 7.29-7.40 (m, 5H), 5.13 (s, 2H), 4.27 (br d, J=2.64 Hz, 2H), 3.79 (tt, J=12.08, 3.54 Hz, 1H), 2.78 (br s, 2H), 2.23-2.39 (m, 1H), 1.85-2.00 (m, 2H), 1.67-1.80 (m, 2H), 1.46 (s, 9H), 0.72-0.78 (m, 2H), 0.60-0.67 (m, 2H).

### Synthesis of B24

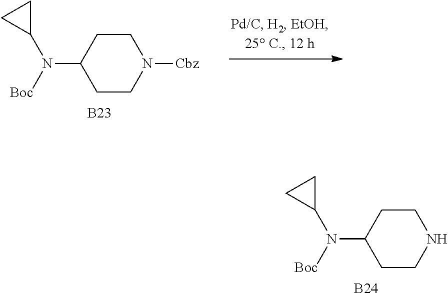

To a solution of benzyl 4-[tert-butoxycarbonyl(cyclopropyl)amino]piperidine-1-carboxylate (B23, 50 g, 134 mmol) in EtOH (2 L) was added Pd/C (10 wt %, 2.8 g, 27 mmol), and the reaction was stirred at 25° C. for 12 h under H₂ (15 Psi). The reaction mixture was filtered through a celite pad, rinsed with EtOH (3×200 mL), and the filtrate was concentrated to afford tert-butyl N-cyclopropyl-N-(4-piperidyl)carbamate (B24, 30 g, 94% yield) as an oil. ¹H NMR (400 MHz, CDCl₃) δ ppm 4.04-4.31 (m, 1H), 3.67-3.85 (m, 1H), 3.11-3.25 (m, 1H), 2.57-2.78 (m, 2H), 2.25-2.41 (m, 1H), 1.90-2.06 (m, 2H), 1.66-1.77 (m, 2H), 1.47 (s, 9H), 0.72-0.80 (m, 2H), 0.62-0.71 (m, 2H).

### Example 60: Synthesis of Compound 284

### Synthesis of B25

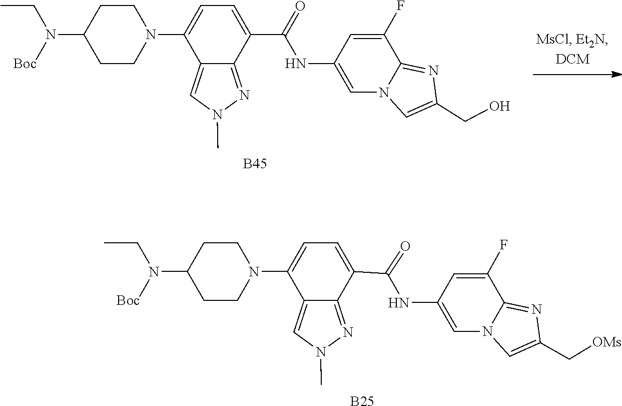

To a solution of tert-butyl N-ethyl-N-[1-[7-[[8-fluoro-2-(hydroxymethyl) imidazo[1,2-a]-pyridin-6-yl]carbamoyl]-2-methyl-indazol-4-yl]-4-piperidyl]carbamate (B45, 130 mg, 530 μmol) in DCM (13 mL) was added N,N-diethylethanamine (80.5 mg, 795 μmol, 110 μL) and methanesulfonyl chloride (60.7 mg, 530 μmol, 41 μL) at 0° C. under N₂. The mixture was stirred at 25° C. for 3 h. The reaction mixture was quenched with saturated aqueous NaHCO₃ (20 mL) and extracted with DCM (3×10 mL). The organic layer was washed with brine (30 mL), dried over Na₂SO₄, filtered and concentrated to afford (6-(4-(4-((tert-butoxycarbonyl)(ethyl)amino)-piperidin-1-yl)-2-methyl-2H-indazole-7-carboxamido)-8-fluoroimidazo[1,2-a]pyridin-2-yl)-methyl methanesulfonate (B25, 130 mg, 61% yield). The crude product was used directly for the next step without further purification. LCMS (ES, m/z): 644.4 [M+H]⁺.

### Synthesis of B26

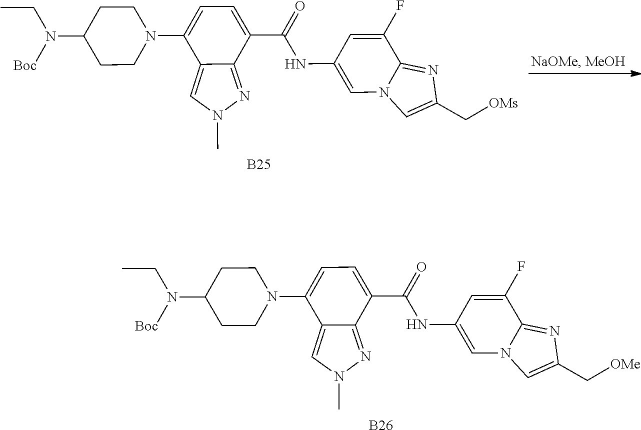

To a solution of tert-butyl N-[1-[7-[[2-(chloromethyl)-8-fluoro-imidazo[1,2-a]pyridin-6-yl]carbamoyl]-2-methyl-indazol-4-yl]-4-piperidyl]-N-ethyl-carbamate (B25, 150 mg, 256 μmol) in MeOH (3.8 mL) was added NaOMe (231 mg, 1.28 mmol, 30% wt) at 0° C. under N₂. The mixture was stirred at 50° C. for 12 h. The reaction mixture was concentrated under reduced pressure and purified by reversed-phase HPLC (Condition 12, Gradient 1) to afford tert-butyl ethyl(1-(7-((8-fluoro-2-(methoxymethyl) imidazo[1,2-a]pyridin-6-yl)carbamoyl)-2-methyl-2H-indazol-4-yl)piperidin-4-yl)carbamate (B26, 45 mg, 78% yield) as a solid. LCMS (ES, m/z): 580.4 [M+H]. ¹H NMR (400 MHz, DMSO-d₆) δ ppm 11.10 (s, 1H), 9.30 (d, J=1.38 Hz, 1H), 8.81 (s, 1H), 8.12 (d, J=2.63 Hz, 1H), 7.98 (d, J=8.13 Hz, 1H), 7.41 (d, J=12.26 Hz, 1H), 6.52 (d, J=8.13 Hz, 1H), 4.52 (s, 2H), 4.31 (s, 3H), 4.00-4.06 (m, 2H), 3.35 (s, 3H), 3.15 (td, J=7.35, 5.32 Hz, 2H), 3.01 (br t, J=11.32 Hz, 2H), 1.84-1.99 (m, 2H), 1.66-1.80 (m, 2H), 1.42 (s, 9H), 1.08 (br t, J=6.63 Hz, 3H). ¹⁹F NMR (376 MHz, DMSO-d₆) δ ppm −131.543.

### Synthesis of Compound 284

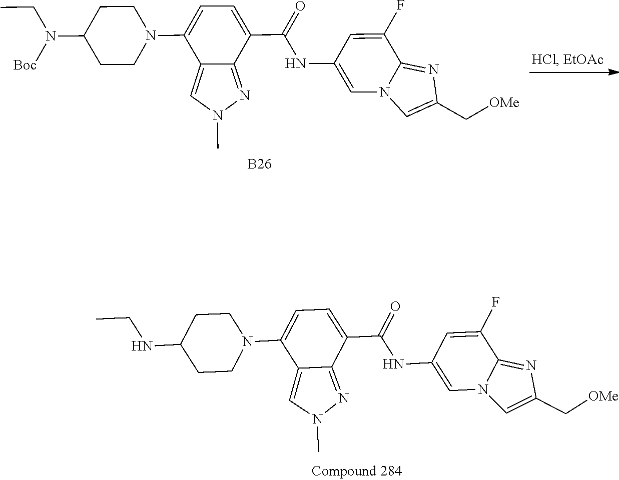

To a solution of tert-butyl N-ethyl-N-[1-[7-[[8-fluoro-2-(methoxymethyl) imidazo[1,2-a]-pyridin-6-yl]carbamoyl]-2-methyl-indazol-4-yl]-4-piperidyl]carbamate (B26, 45.0 mg, 77.6 μmol) in EtOAc (2 mL) was added HCl/EtOAc (4 M, 2 mL), and the reaction was stirred at 25° C. for 2 h. The reaction mixture was concentrated under reduced pressure and purified by prep-HPLC (Condition 11, Gradient 1) to afford 4-[4-(ethylamino)-1-piperidyl]-N-[8-fluoro-2-(methoxymethyl) imidazo[1,2-a]pyridin-6-yl]-2-methyl-indazole-7-carboxamide (Compound 284, 3.75 mg, 10% yield) as a solid. LCMS (ES, m/z): 480.2 [M+H]⁻. ¹H NMR (400 MHz, DMSO-d₆) δ ppm 11.09 (s, 1H), 9.29 (d, J=1.25 Hz, 1H), 8.80 (s, 1H), 8.33 (s, 1H), 8.11 (d, J=2.88 Hz, 1H), 7.97 (d, J=8.13 Hz, 1H), 7.40 (dd, J=12.26, 1.25 Hz, 1H), 6.51 (d, J=8.25 Hz, 1H), 4.51 (s, 2H), 4.30 (s, 3H), 3.95 (br d, J=13.13 Hz, 2H), 3.34 (s, 3H), 3.04 (br t, J=11.69 Hz, 2H), 2.89-2.97 (m, 1H), 2.77 (q, J=7.13 Hz, 2H), 2.04 (br d, J=10.51 Hz, 2H), 1.56 (br d, J=9.76 Hz, 2H), 1.12 (t, J=7.13 Hz, 3H). ¹⁹F NMR (376 MHz, DMSO-d₆) δ ppm −131.539.

### Example 61: Synthesis of B27

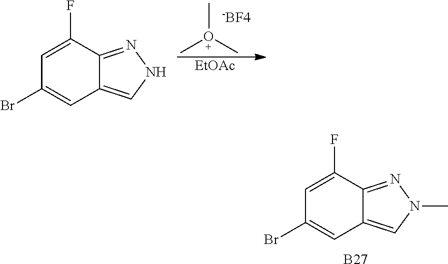

To a solution of 5-bromo-7-fluoro-2H-indazole (1 g, 4.65 mmol) in EtOAc (20 mL) was added trimethyloxonium tetrafluoroborate (1 g, 6.98 mmol) in portions. The mixture was stirred at 25° C. for 12 h. The mixture was diluted with EtOAc (20 mL), washed with brine (50 mL), dried over Na₂SO₄ and filtered. The filtrate was concentrated under reduced pressure and purified by silica gel column chromatography (100-15% EtOAc in PE) to afford 5-bromo-7-fluoro-2-methyl-indazole (B27, 900 mg, 71% yield) as a solid. LCMS (ESI, m/z): 231.0 [M+H]⁺. ¹H NMR (400 MHz, DMSO-d₆) δ ppm 8.47 (d, J=2.75 Hz, 1H), 7.82 (d, J=1.38 Hz, 1H), 7.25 (dd, J=11.01, 1.38 Hz, 1H), 4.20 (s, 3H).

An analogous method was followed to obtain the following compounds.

| Compound | Starting Material | Characterization |
|---|---|---|
| 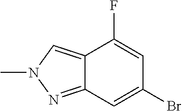 C55 | 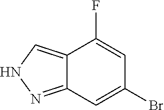 [SMILES: Fc1cc(Br)cc2n[nH]cc12] | Modification: The reaction was ran for 16 h at rt. The residue was purified by silica gel column chromatography (18% EtOAc in PE) to afford 6- bromo-4-fluoro-2-methyl-indazole (C55, 1.5 g, 6.55 mmol, 70% yield) as a solid. LCMS (ES, m/z): 229 [M + H]⁺. |

### Example 62: Synthesis of Compound 192

### Synthesis of B28

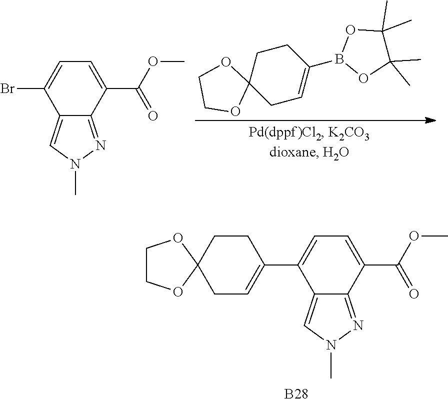

To a mixture of methyl 4-bromo-2-methyl-indazole-7-carboxylate (1 g, 3.7 mmol, 1 eq) and 2-(1,4-dioxaspiro[4.5]dec-7-en-8-yl)-4,4,5,5-tetramethyl-1,3,2-dioxaborolane (989 mg, 3.7 mmol, 1 eq) in 1,4-dioxane (50 mL) and H₂O (10 mL) was added K₂CO₃ (1.5 g, 11 mmol, 3 eq) and dichloro[1,1-bis(diphenylphosphino) ferrocene]palladium (II) dichloromethane adduct (303 mg, 371 μmol, 0.1 eq), and the reaction was stirred at 90° C. for 3 h. The resulting mixture was poured into H₂O (50 mL), extracted with EtOAc (3×50 mL), dried over Na₂SO₄, and filtered. The filtrate was concentrated under reduced pressure and purified by silica gel column chromatography (17% MeOH in EtOAc) to afford methyl 4-(1,4-dioxaspiro[4.5]dec-7-en-8-yl)-2-methylindazole-7-carboxylate (B28, 1.05 g, 77% yield) as an oil. LCMS (ESI, m/z): 329.1 [M+H]⁺.

### Synthesis of B29

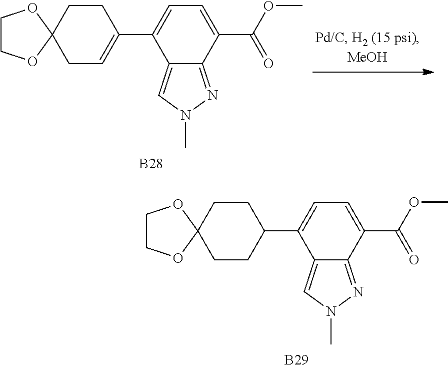

To a suspension of Pd/C (221 mg, 2.1 mmol, 10% w.t., 0.1 eq) in MeOH (15 mL) was added methyl 4-(1,4-dioxaspiro[4.5]dec-7-en-8-yl)-2-methyl-indazole-7-carboxylate (B28, 0.86 g, 2.6 mmol, 1 eq) under N₂. The suspension was degassed under vacuum and purged with H₂ three times. The reaction mixture was stirred at 25° C. for 12 h under H₂ (15 psi). The reaction mixture was filtered through a pad of celite and the filter cake was washed with MeOH (40 mL). The filtrate was concentrated under reduced pressure to afford methyl 4-(1,4-dioxaspiro[4.5]decan-8-yl)-2-methyl-indazole-7-carboxylate (B29, 0.86 g, 90% yield) as an oil. The material was used to next step without any further purification. LCMS (ESI, m/z): 331.1 [M+H]⁺. ¹H NMR (400 MHz, CDCl₃) δ ppm 8.10 (s, 1H), 8.04 (d, J=7.40 Hz, 1H), 7.01 (d, J=7.40 Hz, 1H), 4.32 (s, 3H), 4.01 (d, J=2.64 Hz, 7H), 2.77-3.09 (m, 1H), 1.87-2.03 (m, 6H), 1.68-1.81 (m, 2H).

### Synthesis of B30

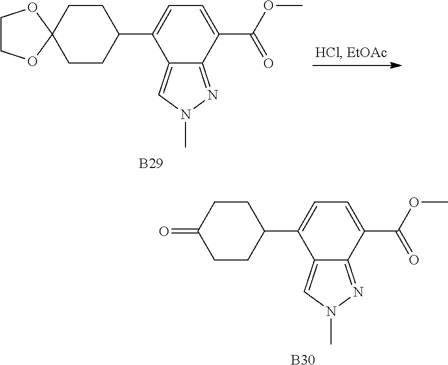

The mixture of methyl 4-(1,4-dioxaspiro[4.5]decan-8-yl)-2-methyl-indazole-7-carboxylate (B29, 0.86 g, 2.6 mmol) in HCl/EtOAc (4 M, 15 mL) was stirred at 25° C. for 12 h. One additional vial was set up as described above and all two reaction mixtures were combined. The reaction mixture was poured into water (20 mL), extracted with EtOAc (3×15 mL), dried over Na₂SO₄, and filtered. The filtrate was concentrated under reduced pressure and purified by silica gel column chromatography (100% EtOAc) to afford methyl 2-methyl-4-(4-oxocyclohexyl)indazole-7-carboxylate (B30, 0.55 g, 54% yield) as an oil. LCMS (ESI, m/z): 287.1 [M+H]⁺. ¹H NMR (400 MHz, CDCl₃) δ ppm 8.09-8.14 (m, 1H), 8.03-8.08 (m, 1H), 7.00 (d, J=7.38 Hz, 1H), 4.30-4.37 (m, 3H), 3.99-4.06 (m, 3H), 3.32-3.44 (m, 1H), 2.54-2.63 (m, 4H), 2.31-2.42 (m, 2H), 2.01-2.21 (m, 2H).

### Synthesis of B31

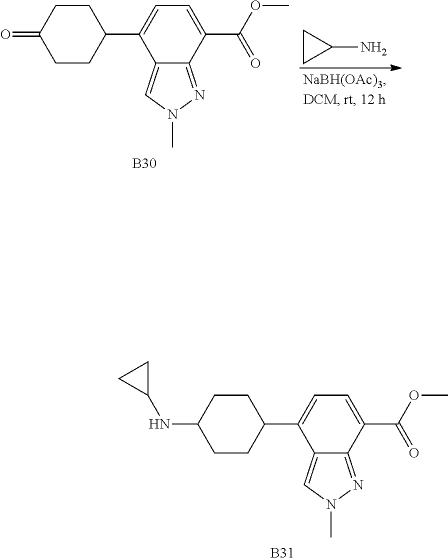

The mixture of methyl 2-methyl-4-(4-oxocyclohexyl)indazole-7-carboxylate (B30, 50 mg, 174 μmol, 1 eq), cyclopropanamine (11 mg, 192 μmol, 13 μL, 1.1 eq) and NaBH(OAc)₃ (81 mg, 384 μmol, 2.2 eq) in DCM (0.5 mL) was stirred at 25° C. for 12 h. The reaction mixture was poured into saturated aqueous NaHCO₃ solution (2 mL) and stirred for 10 mins. The organic layer was dried over Na₂SO₄, filtered and concentrated under reduced pressure to afford methyl 4-[4-(cyclopropylamino)cyclohexyl]-2-methyl-indazole-7-carboxylate (B31, 25 mg, crude) as a solid. The material was used to next step without any further purification. LCMS (ESI, m/z): 328.1 [M+H]⁺.

### Synthesis of B32

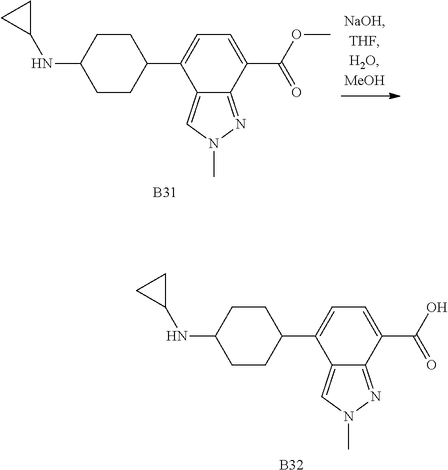

To a mixture of methyl 4-[4-(cyclopropylamino)cyclohexyl]-2-methyl-indazole-7-carboxylate (B31, 0.55 g, 1.68 mmol, 1 eq) in THE (2.2 mL), H₂O (2.2 mL) and MeOH (0.6 mL) was added NaOH (335 mg, 8.4 mmol, 5 eq), and the reaction was stirred at 50° C. for 12 h. One additional vial was set up as described above and all two reaction mixtures were combined. The reaction mixture adjusted pH=6 by addition of aqueous HCl (1 M, 10 mL). Then the reaction mixture was filtered. The filter cake was dried in vacuum to afford 4-[4-(cyclopropylamino)cyclohexyl]-2-methyl-indazole-7-carboxylic acid (B32, 1.1 g, crude) as a solid. LCMS (ESI, m/z): 314.2 [M+H]⁺.

### Synthesis of Compound 192

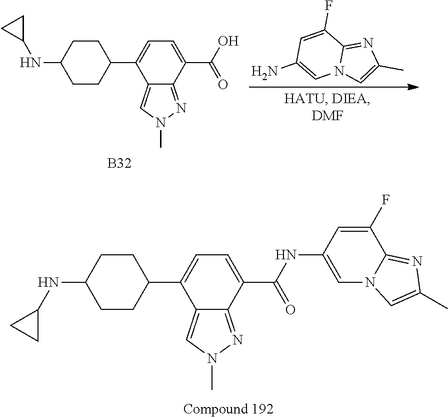 [SMILES: "B32" ; Cn1cc2c(C3CCC(NC4CC4)CC3)ccc(C(=O)O)c2n1 ; Cc1cn2cc(NC(=O)c3ccc(C4CCC(NC5CC5)CC4)c4cn(C)nc34)cc(F)c2n1 ; Cc1cn2cc(N)cc(F)c2n1]

To a mixture of 4-[4-(cyclopropylamino)cyclohexyl]-2-methyl-indazole-7-carboxylic acid (B32, 0.10 g, 319 μmol, 1 eq) and 2-(3H-[1,2,3]triazolo[4,5-b]pyridin-3-yl)-1,1,3,3-tetramethyluronium hexafluorophosphate (145 mg, 382 μmol, 1.2 eq) in N,N-dimethyl formamide (1.4 mL) was added N-ethyl-N-isopropylpropan-2-amine (103 mg, 797 μmol, 138 μL), and the reaction was stirred at 25° C. for 30 mins. A mixture of 8-fluoro-2-methyl-imidazo[1,2-a]pyridin-6-amine (68.5 mg, 414 μmol, 1.3 eq) and N-ethyl-N-isopropylpropan-2-amine (103 mg, 797 μmol, 138 μL) in N,N-dimethylformamide (1.4 mL) was then added to above reaction mixture, and the reaction was stirred at 50° C. for 12 h. The resulting mixture was diluted with EtOAc (50 mL), washed with brine (3×10 mL), dried over Na₂SO₄, and filtered. The filtrate was concentrated under reduced pressure and purified by prep-HPLC (Condition 9) to get 4-[4-(cyclopropylamino)cyclohexyl]-N-(8-fluoro-2-methyl-imidazo[1,2-a]pyridin-6-yl)-2-methyl-indazole-7-carboxamide (Compound 192, 7 mg, 5% yield) as a solid. LCMS (ESI, m/z): 461.3 [M+H]⁺. ¹H NMR (400 MHz, Methanol-d₄) δ ppm 9.59 (d, J=1.00 Hz, 1H), 8.71-8.74 (m, 1H), 8.22 (d, J=7.38 Hz, 1H), 8.09-8.16 (m, 2H), 7.27 (d, J=7.38 Hz, 1H), 4.40 (s, 3H), 3.63-3.65 (m, 1H), 3.23-3.30 (m, 1H), 2.84-2.88 (m, 1H), 2.59 (s, 3H), 1.95-2.26 (m, 8H), 0.99 (d, J=5.63 Hz, 4H).

### Example 63: Synthesis of Compound 189

### Synthesis of B33

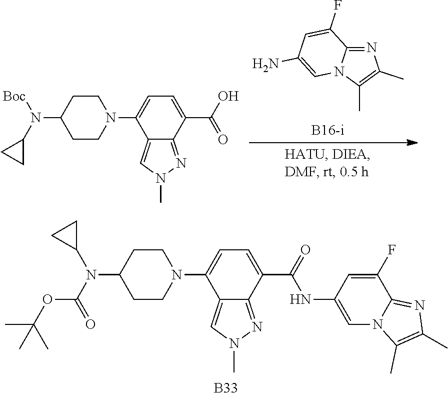

A mixture of 4-[4-[tert-butoxycarbonyl(cyclopropyl)amino]-1-piperidyl]-2-methyl-indazole-7-carboxylic acid (100 mg, 241 μmol, 1 eq), N,N-diisopropylethylamine (70.8 mg, 548 μmol, 95.5 μL, 2.5 eq) and o-(7-Azabenzotriazol-1-yl)-N,N,N,N-tetramethyluronium hexafluorophosphate (100 mg, 263 μmol, 1.2 eq) in N,N-dimethylformamide (0.2 mL) was stirred at 25° C. for 30 mins. Then, a mixture of 8-fluoro-2,3-dimethyl-imidazo[1,2-a]pyridin-6-amine (B16-i, 43.2 mg, 241 μmol, 1.10 eq) and N,N-diisopropylethylamine (70.8 mg, 548 μmol, 95.5 μL, 2.5 eq) in N,N-dimethylformamide (0.2 mL) was added to the reaction mixture. The reaction mixture was stirred at 50° C. for 12 h. The reaction mixture was diluted with water (10 mL), extracted with EtOAc (3×10 mL), washed with brine (2×20 mL), dried over Na₂SO₄, and filtered. The filtrate was concentrated under reduced pressure and purified by prep-HPLC (Condition 9, Gradient 6) to give tert-butyl N-cyclopropyl-N-[1-[7-[(8-fluoro-2,3-dimethyl-imidazo[1,2-a]pyridin-6-yl)carbamoyl]-2-methyl-indazol-4-yl]-4-piperidyl]carbamate (B33, 55 mg, 40% yield) as a solid. LCMS (ESI, m/z): 576.4 [M+H]⁺. ¹H NMR (400 MHz, Methanol-d₄) δ 9.27 (s, 1H), 8.55 (s, 1H), 8.10 (d, J=8.3 Hz, 1H), 7.94 (br d, J=11.3 Hz, 1H), 6.55 (d, J=8.3 Hz, 1H), 4.34 (s, 3H), 4.13 (br d, J=12.9 Hz, 2H), 3.82 (tt, J=4.0, 12.0 Hz, 1H), 3.12-3.03 (m, 2H), 2.57 (s, 3H), 2.52 (s, 3H), 2.33 (dq, J=3.8, 12.4 Hz, 2H), 1.88 (br d, J=9.8 Hz, 2H), 1.45 (s, 9H), 0.86-0.78 (m, 2H), 0.74-0.64 (m, 2H).

### Synthesis of Compound 189

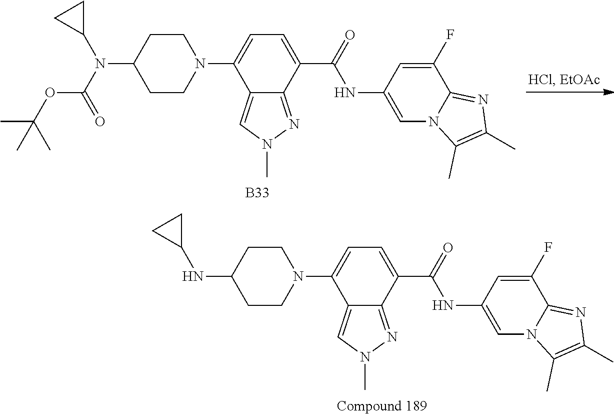

To a solution of tert-butyl N-cyclopropyl-N-[1-[7-[(8-fluoro-2,3-dimethyl-imidazo[1,2-a]pyridin-6-yl)carbamoyl]-2-methyl-indazol-4-yl]-4-piperidyl]carbamate (B33, 50 mg, 86.8 μmol, 1 eq) in EtOAc (1 mL) was added HCl/EtOAc (4 M, 977 μL), and the reaction was stirred at 25° C. for 1 h. The reaction mixture was concentrated under reduced pressure to afford 4-[4-(cyclopropylamino)-1-piperidyl]-N-(8-fluoro-2,3-dimethyl-imidazo[1,2-a]pyridin-6-yl)-2-methyl-indazole-7-carboxamide (Compound 189, 22 mg, 52% yield) as a solid. LCMS (ESI, m/z): 476.3 [M+H]⁺. ¹H NMR (400 MHz, Methanol-d₄) δ 9.32 (d, J=1.0 Hz, 1H), 8.67 (s, 1H), 8.19-8.12 (m, 1H), 8.06 (dd, J=1.3, 11.3 Hz, 1H), 6.64 (d, J=8.1 Hz, 1H), 4.37 (s, 3H), 4.17 (br d, J=13.4 Hz, 2H), 3.58 (tt, J=4.1, 11.6 Hz, 1H), 3.22-3.10 (m, 2H), 2.86 (tt, J=3.8, 7.3 Hz, 1H), 2.56 (d, J=18.1 Hz, 6H), 2.37 (br d, J=10.1 Hz, 2H), 1.92 (dq, J=3.9, 12.2 Hz, 2H), 1.05-0.87 (m, 4H).

An analogous method was followed to obtain the following compounds.

| | Starting | |
|---|---|---|
| Compound | Material | Characterization |
| 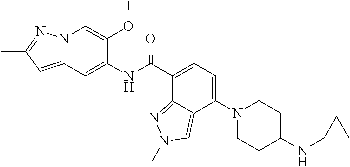 [SMILES: COc1cn2nc(C)cc2cc1NC(=O)c1ccc(N2CCC(NC3CC3)CC2)c2cn(C)nc12] Compound 475 | 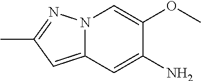 | Modification: Step 1 was ran for 3 h at room temperature. Step 2 was ran using TFA in DCM. The crude product was purified by Prep- HPLC (Condition 5, Gradient 23) to afford 4- [4-(cyclopropylamino)-1-piperidyl]-N-(6- methoxy-2-methyl-pyrazolo[1,5-a]pyridin- 5-yl)-2-methyl-indazole-7-carboxamide (Compound 475, 40 mg, 84.5 μmol). LCMS (ES, m/z): 474 [M + H]⁺. ¹H NMR (300 MHz, DMSO-d₆) δ 11.72 (s, 1H), 8.77 (s, 1H), 8.64 (s, 1H), 8.37 (s, 1H), 8.00 (d, J = 8.1 Hz, 1H), 6.49 (d, J = 8.2 Hz, 1H), 6.23 (s, 1H), 4.28 (s, |
| | | 3H), 4.04 (s, 3H), 3.89 (d, J = 12.9 Hz, 2H), |
| | | 3.07 (t, J = 11.8 Hz, 2H), 2.86-2.64 (m, 1H), |
| | | 2.33 (s, 3H), 2.28 (s, 1H), 2.12 (tt, J = 6.6, 3.6 |
| | | Hz, 1H), 2.01 (d, J = 12.5 Hz, 2H), 1.60-1.33 |
| | | (m, 2H), 0.40 (dt, J = 6.2, 3.0 Hz, 2H), 0.24 |
| | | (q, J = 3.2, 2.7 Hz, 2H). |

### Example 64: Synthesis of Compound 642

### Synthesis of B34

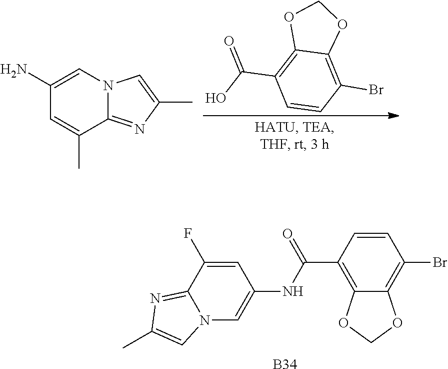

A solution of 8-fluoro-2-methyl-imidazo[1,2-a]pyridin-6-amine (235.9 mg, 1.4 mmol) and 7-bromo-1,3-benzodioxole-4-carboxylic acid (350 mg, 1.4 mmol) and TEA (433.6 mg, 4.3 mmol, 597.3 μL) and HATU (543.1 mg, 1.4 mmol) in THF (5 mL) was stirred for 3 h at room temperature. The reaction was quenched with water (2 mL) at room temperature. The resulting mixture was extracted with EtOAc (3×5 mL). The combined organic layers were dried over anhydrous Na₂SO₄, and filtered. The filtrate was concentrated under reduced pressure and purified by silica gel column chromatography (89% EtOAc in PE) to afford 7-bromo-N-(8-fluoro-2-methyl-imidazo[1,2-a]pyridin-6-yl)-1,3-benzodioxole-4-carboxamide (B34, 400 mg, 1.02 mmol, 71% yield) as a solid. LCMS (ESI, m/z): 392 [M+H]⁺.

### Synthesis of B35

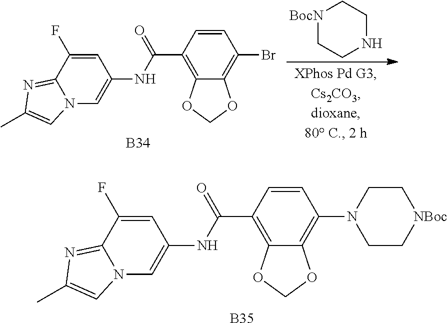

A solution of tert-butyl piperazine-1-carboxylate (47.5 mg, 255 μmol), 7-bromo-N-(8-fluoro-2-methyl-imidazo[1,2-a]pyridin-6-yl)-1,3-benzodioxole-4-carboxamide (100 mg, 255 μmol), XPhos Pd G3 (21.6 mg, 25.5 μmol) and Cs₂CO₃ (249.2 mg, 765 μmol) in dioxane (2 mL) was stirred for 2 h at 80° C. under N₂ atmosphere. The resulting mixture was concentrated under reduced pressure and purified by Prep-TLC (50% EtOAc in PE) to afford tert-butyl 4-[7-[(8-fluoro-2-methyl-imidazo[1,2-a]pyridin-6-yl)carbamoyl]-1,3-benzodioxol-4-yl]piperazine-1-carboxylate (80 mg, 160.8 μmol, 63% yield) as a solid. LCMS (ESI, m/z): 498 [M+H]⁺.

### Synthesis of Compound 642

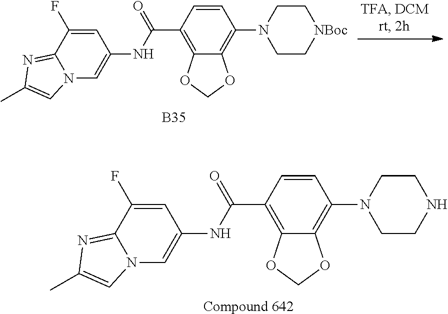

A solution of tert-butyl 4-[7-[(8-fluoro-2-methyl-imidazo[1,2-a]pyridin-6-yl)carbamoyl]-1,3-benzodioxol-4-yl]piperazine-1-carboxylate (80 mg, 160.8 μmol) and TFA (745 mg, 6.5 mmol, 0.5 mL) in DCM (3 mL) was stirred for 2 h at room temperature. The resulting mixture was concentrated under reduced pressure and purified by Prep-HPLC (Condition 7, Gradient 1) to afford N-(8-fluoro-2-methyl-imidazo[1,2-a]pyridin-6-yl)-7-piperazin-1-yl-1,3-benzodioxole-4-carboxamide (Compound 642, 7.5 mg, 18.8 μmol, 12% yield, 99% purity) as a solid. LCMS (ESI, m/z): 398 [M+H]⁺. ¹H NMR (400 MHz, DMSO-d₆, ppm) δ 9.58 (s, 1H), 9.05 (d, J=1.7 Hz, 1H), 7.88 (dd, J=3.2, 1.0 Hz, 1H), 7.30-7.22 (m, 2H), 6.59 (d, J=8.9 Hz, 1H), 6.10 (s, 2H), 3.18 (dd, J=6.2, 3.7 Hz, 4H), 2.84 (dd, J=6.2, 3.5 Hz, 4H), 2.36-2.31 (m, 3H).

An analogous method was followed to obtain the following compounds.

| | Starting | |
|---|---|---|
| Compound | Material | Characterization |
| 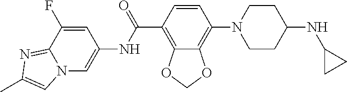 [SMILES: Cc1cn2cc(NC(=O)c3ccc(N4CCC(NC5CC5)CC4)c4c3OCO4)cc(F)c2n1] Compound 648 | 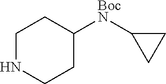 [SMILES: CC(C)(C)OC(=O)N(C1CCNCC1)C1CC1] | The crude product was purified by Prep-HPLC (Condition 8, Gradient 1) to afford 7-[4-(cyclopropylamino)-1- piperidyl]-N-(8-fluoro-2-methyl- imidazo[1,2-a]pyridin-6-yl)-1,3- benzodioxole-4-carboxamide (Compound 648, 3.4 mg, 7.4 μmol, 5% yield, 98% purity) as a solid. LCMS (ESI, m/z): 452 [M + H]⁺. ¹H NMR (400 MHz, DMSO-d₆, ppm) δ 9.55 (s, 1H), 9.04 (d, J = 1.7 Hz, 1H), 7.91-7.85 (m, |
| | | 8.9 Hz, 1H), 6.10 (s, 2H), 3.71 (dd, J = |
| | | 10.3, 6.7 Hz, 2H), 2.91-2.80 (m, 2H), |
| | | 2.70 (d, J = 11.0 Hz, 1H), 2.36-2.31 |
| | | (m, 3H), 2.11 (tt, J = 6.6, 3.5 Hz, 1H), |
| | | 1.92 (dd, J = 12.9, 3.8 Hz, 2H), 1.44- |
| | | 1.29 (m, 2H), 0.38 (td, J = 6.4, 4.1 Hz, |
| | | 2H), 0.26-0.18 (m, 2H). |

### Example 65: Synthesis of Compound 643

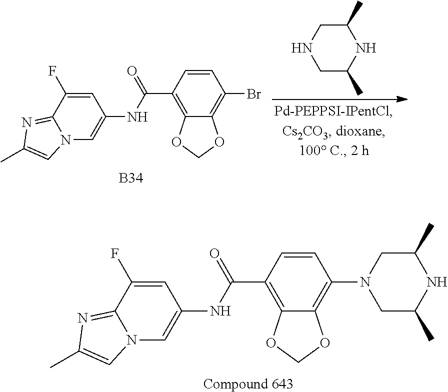

A solution of 7-bromo-N-(8-fluoro-2-methyl-imidazo[1,2-a]pyridin-6-yl)-1,3-benzodioxole-4-carboxamide (0.1 g, 255 μmol), (2S,6R)-2,6-dimethylpiperazine (43.7 mg, 382.5 μmol), Cs₂CO₃ (249.2 mg, 765 μmol), Pd-PEPPSI-IPentCl (21.4 mg, 25.5 μmol) in dioxane (1 mL) was stirred for 2 h at 100° C. under N₂. The resulting mixture was concentrated under reduced pressure and purified by Prep-HPLC (Condition 8, Gradient 3) to afford 7-[(3S,5R)-3,5-dimethylpiperazin-1-yl]-N-(8-fluoro-2-methyl-imidazo[1,2-a]pyridin-6-yl)-1,3-benzodioxole-4-carboxamide (Compound 643, 7.2 mg, 16.8 μmol, 7% yield, 99% purity). LCMS (ESI, m/z): 426 [M+H]⁺. ¹H NMR (400 MHz, DMSO-d₆, ppm) δ 9.55 (s, 1H), 9.04 (d, J=1.7 Hz, 1H), 7.88 (dd, J=3.2, 1.0 Hz, 1H), 7.30-7.22 (m, 2H), 6.59 (d, J=8.9 Hz, 1H), 6.10 (s, 2H), 3.67-3.59 (m, 2H), 2.85 (s, 2H), 2.36-2.31 (m, 3H), 2.26 (t, J=11.0 Hz, 2H), 1.00 (d, J=6.3 Hz, 6H). ¹⁹F NMR (376 MHz, DMSO-d₆, ppm) δ −132.20.

An analogous method was followed to obtain the following compounds.

| Compound | Starting Material | Characterization |
|---|---|---|
| 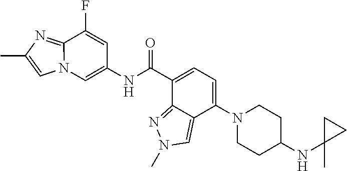 [SMILES: Cc1cn2cc(NC(=O)c3ccc(N4CCC(NC5(C)CC5)CC4)c4cn(C)nc34)cc(F)c2n1] Compound 355 | 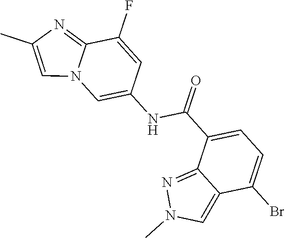 A39 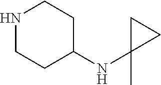 C107 | Modification: The reaction was ran at 90° C. The residue was purified by silica gel column chromatography (9% MeOH in DCM) to afford N-(8-fluoro-2- methyl-imidazo[1,2-a]-pyridin- 6-yl)-2-methyl-4-[4-[(1-methyl- cyclopropyl)amino]-1- piperidyl]indazole-7- carboxamide (Compound 355, 28.8 mg, 60.6 μmol, 20% yield) as a solid. LCMS (ES, m/z): 476 [M + H]⁺. ¹H NMR (300 MHz, DMSO-d₆) δ 11.07 (s, 1H), 9.22 (d, J = 1.6 Hz, 1H), 8.80 (s, 1H), 7.97 (d, J = 8.1 Hz, 1H), 7.91 (d, J = 3.1 Hz, 1H), 7.34 (dd, J = 12.4, 1.7 Hz, 1H), 6.50 (d, J = 8.2 Hz, 1H), 4.30 (s, 3H), 3.91 (d, J = 12.6 Hz, 2H), 3.17-2.95 (m, 3H), 2.35 (s, 3H), 2.19-2.11 (m, 1H), 2.00 (d, J = 6.2 Hz, 2H), 1.49-1.35 (m, 2H), 1.24 (s, 3H), 0.47 (s, 2H), 0.32 (s, 2H). |

### Example 66: Synthesis of Compound 649

### Synthesis of B36

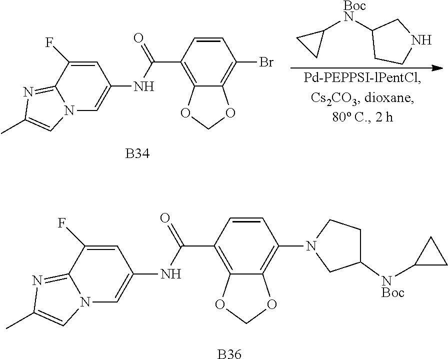

Solution of 7-bromo-N-(8-fluoro-2-methyl-imidazo[1,2-a]pyridin-6-yl)-1,3-benzodioxole-4-carboxamide (B34, 0.1 g, 255 μmol), tert-butyl N-cyclopropyl-N-pyrrolidin-3-yl-carbamate (86.6 mg, 382.5 μmol), Cs₂CO₃ (249.2 mg, 765 μmol), Pd-PEPPSI-IPentCl (21.4 mg, 25.5 μmol) in dioxane (1 mL) was stirred for 2 h at 80° C. The resulting mixture was concentrated under reduced pressure and purified by silica gel column chromatography (25% EtOAc in PE) to afford tert-butyl cyclopropyl(1-(7-((8-fluoro-2-methylimidazo[1,2-a]pyridin-6-yl)-carbamoyl)-benzo[d][1,3]dioxol-4-yl)pyrrolidin-3-yl)carbamate (B36, 0.1 g, 56% yield) as a solid. LCMS (ESI, m/z): 538 [M+H]⁺.

### Synthesis of Compound 649

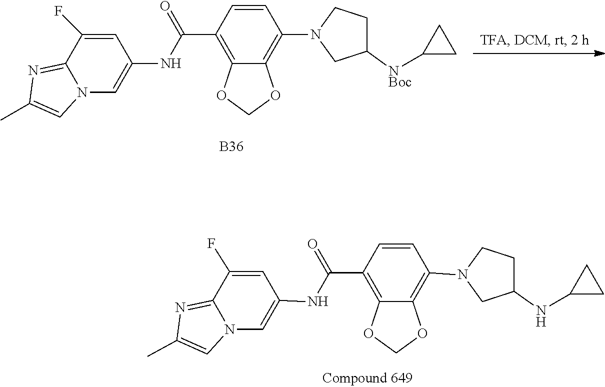

A solution of tert-butyl cyclopropyl(1-(7-((8-fluoro-2-methylimidazo[1,2-a]pyridin-6-yl)carbamoyl)benzo[d][1,3]dioxol-4-yl)pyrrolidin-3-yl)carbamate (B36, 100 mg, 382.5 μmol), CF₃COOH (1 mL) in DCM (3 mL) was stirred for 2 h at 25° C. under air atmosphere. The resulting mixture was concentrated under reduced pressure and purified by Prep-HPLC (Condition 8, Gradient 2) to afford 7-[3-(cyclopropylamino)-pyrrolidin-1-yl]-N-(8-fluoro-2-methyl-imidazo[1,2-a]pyridin-6-yl)-1,3-benzodioxole-4-carboxamide (Compound 649, 1.8 mg, 4.1 μmol, 2% yield, 99% purity). LCMS (ESI, m/z): 438 [M+H]⁺. ¹H NMR (400 MHz, DMSO-d₆, ppm) δ 9.34 (s, 1H), 9.04 (d, J=1.6 Hz, 1H), 7.87 (d, J=3.0 Hz, 1H), 7.32-7.23 (m, 2H), 6.29 (d, J=8.9 Hz, 1H), 6.05-6.00 (m, 2H), 3.65 (dd, J=10.0, 6.0 Hz, 2H), 3.59-3.42 (m, 2H), 3.42-3.30 (m, 1H), 2.45 (s, 4H), 2.08 (s, 2H), 1.82 (dd, J=12.2, 6.5 Hz, 1H), 0.40 (d, J=6.9 Hz, 2H), 0.24 (s, 2H). ¹⁹F NMR (376 MHz, DMSO-d₆, ppm) δ −132.34.

### Example 67: Synthesis of Compound 650

### Synthesis of B37

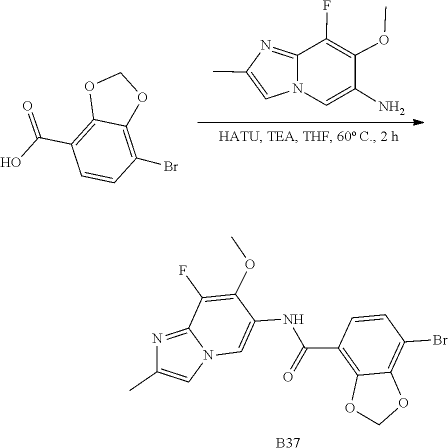

A solution of 8-fluoro-7-methoxy-2-methyl-imidazo[1,2-a]pyridin-6-amine (79.7 mg, 408.1 μmol), 7-bromo-1,3-benzodioxole-4-carboxylic acid (100 mg, 408.1 μmol). TEA (123.9 mg, 1.22 mmol, 170.7 μL) and HATU (310.4 mg, 816.2 μmol) in THF (5 mL) was stirred for 2 h at 60° C. The residue was dissolved in EtOAc (50 mL). The organic layers were washed with H₂O (2×50 mL), dried over anhydrous Na₂SO₄, filtered, and concentrated under reduced pressure to afford 7-bromo-N-(8-fluoro-7-methoxy-2-methylimidazo[1,2-a]pyridin-6-yl)-1,3-benzodioxole-4-carboxamide (B37, 80 mg, 189.5 μmol, 46% yield) as a solid. LCMS (ESI, m/z): 422 [M+H]⁺.

### Synthesis of B38

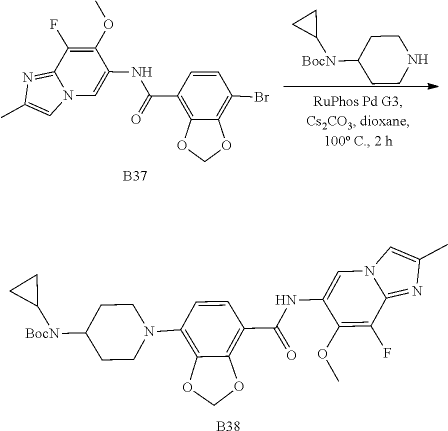

A solution of N-(8-fluoro-7-methoxy-2-methyl-imidazo[1,2-a]pyridin-6-yl)-1,3-benzodioxole-4-carboxamide (50 mg, 145.6 μmol), tert-butyl N-cyclopropyl-N-(4-piperidyl)carbamate (35 mg, 145.6 μmol), RuPhos Pd G3 (12.2 mg, 14.6 μmol) and Cs₂CO₃ (142.4 mg, 436.9 μmol) in Dioxane (2 mL) was stirred for 2 h at 100° C. under N₂. The resulting mixture was filtered and the filter cake was washed with Dioxane (5 mL). The filtrate was concentrated under reduced pressure to afford the crude product tert-butyl N-cyclopropyl N-[1-[7-[(8-fluoro-7-methoxy-2-methyl-imidazo[1,2-a]pyridin-6-yl)carbamoyl]-1,3-benzodioxol-4-yl]-4-piperidyl]carbamate (B38, 50 mg, 86 μmol, 59% yield) as a solid, which was used in the next step directly without further purification. LCMS (ESI, m/z): 582 [M+H]⁺.

### Synthesis of Compound 650

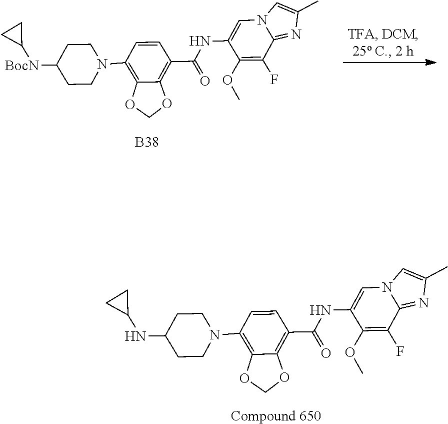

A solution of tert-butyl N-cyclopropyl-N-[1-[7-[(8-fluoro-7-methoxy-2-methyl-imidazo-[1,2-a]pyridin-6yl)carbamoyl]-1,3-benzodioxol-4-yl]-4-piperidyl]carbamate (50 mg, 86 μmol) and TFA (9.8 mg, 86 μmol, 6.6 μL) in DCM (1 mL) was stirred for 2 h at 25° C. The resulting mixture was concentrated under reduced pressure and purified by Prep-HPLC (Condition 8, Gradient 2) to afford 7-[4-(cyclopropylamino)-1-piperidyl]-N-(8-fluoro-7-methoxy-2-methyl-imidazo[1,2-a]pyridin-6-yl)-1,3-benzodioxole-4-carboxamide (Compound 650, 6.6 mg, 13.5 μmol, 16% yield, 99% purity) as a solid. LCMS (ESI, m/z): 482 [M+H]⁺. ¹H NMR (400 MHz, Methanol-d₄, ppm) δ 9.22 (s, 1H), 7.51 (s, 1H), 7.43 (d, J=8.8 Hz, 1H), 6.59 (d, J=8.7 Hz, 1H), 6.16 (s, 2H), 4.20 (d, J=2.7 Hz, 3H), 3.90 (d, J=12.7 Hz, 2H), 2.85 (t, J=12.6 Hz, 3H), 2.35 (s, 3H), 2.24 (s, 1H), 2.05 (d, J=13.2 Hz, 2H), 1.51 (dd, J=13.2, 9.5 Hz, 2H), 0.53 (dd, J=7.1, 4.9 Hz, 2H), 0.44-0.36 (m, 2H).

An analogous method was followed to obtain the following compounds.

| | Starting | |
|---|---|---|
| Compound | Material | Characterization |
| 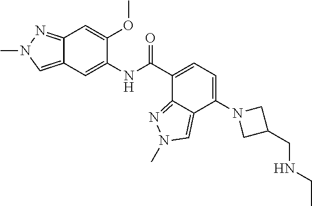 [SMILES: CCNCC1CN(c2ccc(C(=O)Nc3cc4cn(C)nc4cc3OC)c3nn(C)cc23)C1] | 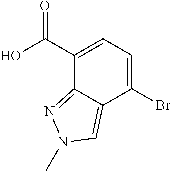 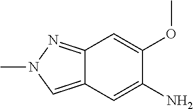 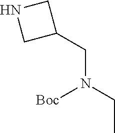 | Modifications: Step 1 was ran in DIEA and DMF at room temperature for 3 h. Step 2 was ran at 90° C. Step 3 was ran for 1 h. The residue was purified by reversed- phase flash chromatography (Condition 3, Gradient 26) to afford 4-[3- (ethylaminomethyl)azetidin-1-yl]-N-(6- methoxy-2-methyl-indazol-5-yl)-2- methyl-indazole-7-carboxamide (Compound 493, 13.5 mg, 30.1 μmol). LCMS (ES, m/z): 448 [M + H]⁺. ¹H NMR (300 MHz, DMSO-d₆) δ 11.46 (s, 1H), 8.82 (s, 1H), 8.63 (s, 1H), 8.16 (s, 1H), 7.94 (d, J = 8.0 Hz, 1H), 7.05 (s, 1H), 5.85 (d, J = 8.1 Hz, 1H), 4.30 (d, J = 8.1 Hz, 2H), 4.26 (s, 3H), 4.08 (s, 3H), 4.04 (s, 3H), 3.92 (dd, J = 8.3, 5.1 Hz, 2H), 2.90 (s, 1H), 2.81 (d, J = 7.1 Hz, 2H), 2.58 (q, J = 7.1 Hz, 2H), 1.03 (t, J = 7.1 Hz, 3H). |

### Example 68: Synthesis of Compound 202

### Synthesis of B39

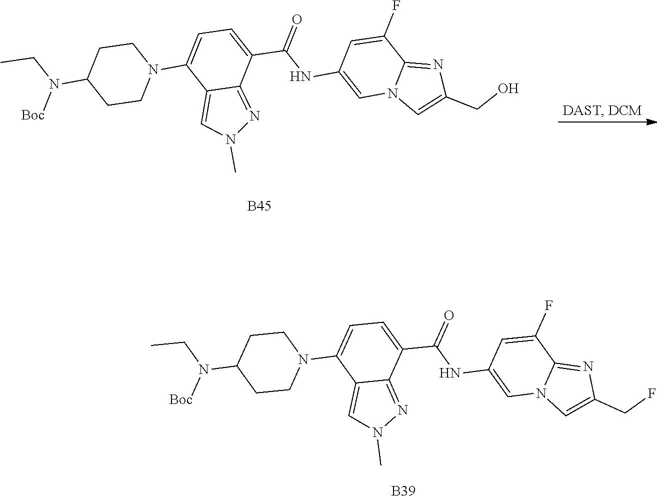

To a solution of tert-butyl N-ethyl-N-[1-[7-[[8-fluoro-2-(hydroxymethyl) imidazo[1,2-a]-pyridin-6-yl]carbamoyl]-2-methyl-indazol-4-yl]-4-piperidyl]carbamate (B45, 100 mg, 176 μmol) in DCM (3 mL) was added N-ethyl-N-(trifluoro-sulfanyl) ethanamine (31.3 mg, 194 μmol, 25.6 μL) at 0° C. under N₂, and the mixture was stirred at 0° C. for 1 h. The reaction mixture was poured into water (20 mL) and extracted with EtOAc (3×20 mL). The combined organic phase was washed with brine (60 mL), dried with anhydrous Na₂SO₄, and filtered. The filtrate was concentrated under reduced pressure and purified by silica gel column chromatography (50% EtOAc in PE) to give tert-butyl N-ethyl-N-[1-[7-[[8-fluoro-2-fluoromethyl) imidazo[1,2-a]-pyridin-6-yl]carbamoyl]-2-methyl-indazol-4-yl]-4-piperidyl]carbamate (B39, 50 mg, 50% yield) as a solid. LCMS (ESI, m/z): 568.5 [M+H]⁺. ¹H NMR (400 MHz, DMSO-d₆) δ ppm 11.12 (s, 1H), 9.35 (s, 1H), 8.80 (s, 1H), 8.32 (t, J=3.06 Hz, 1H), 7.98 (d, J=8.00 Hz, 1H), 7.47 (d, J=12.38 Hz, 1H), 6.52 (d, J=8.13 Hz, 1H), 5.55 (s, 1H), 5.43 (s, 1H), 4.30 (s, 3H), 3.92-4.15 (m, 3H), 3.12-3.22 (m, 2H), 3.01 (br t, J=12.01 Hz, 2H), 1.87-1.99 (m, 2H), 1.64-1.79 (m, 2H), 1.41 (s, 9H), 1.05-1.11 (m, 3H). ¹⁹F NMR (376 MHz, DMSO-d₆) δ ppm −131.11 (s, 1F) −204.33 (s, 1F).

### Synthesis of Compound 202

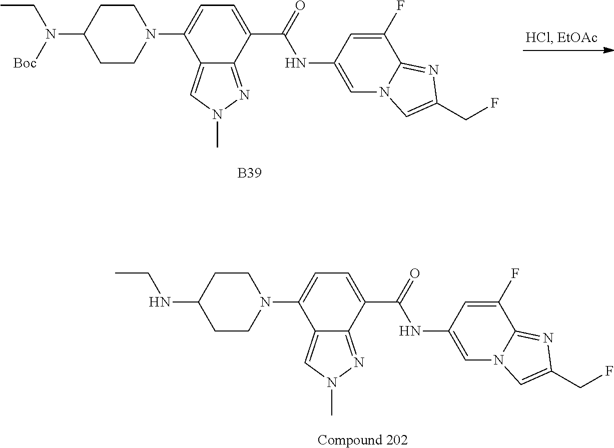

To a solution of tert-butyl N-ethyl-N-[1-[7-[[8-fluoro-2-(fluoromethyl) imidazo[1,2-a]-pyridin-6-yl]carbamoyl]-2-methyl-indazol-4-yl]-4-piperidyl]carbamate (B39, 50 mg, 88 μmol) in EtOAc (1 mL) was added HCl (4 M, 1 mL), and the reaction was stirred at 25° C. for 2 h. The mixture was concentrated under reduced pressure and triturated with DCM (1 mL). The mixture was filtered and the filter cake was concentrated under reduced pressure to afford 4-[4-(ethylamino)-1-piperidyl]-N-[8-fluoro-2-(fluoromethyl) imidazo[1,2-a]pyridin-6-yl]-2-methyl-indazole-7-carboxamide (Compound 202, 46 mg, 25%, as HCl salt) as a solid. LCMS (ESI, m/z): 468.2 [M+H]⁺. ¹H NMR (400 MHz, Methanol-d₄) δ ppm 9.63 (s, 1H), 8.59 (s, 1H), 8.48 (d, J=2.50 Hz, 1H), 8.06-8.23 (m, 2H), 6.62 (d, J=8.25 Hz, 1H), 5.74 (s, 1H), 5.62 (s, 1H), 4.36 (s, 3H), 4.15 (br d, J=13.01 Hz, 2H), 3.38-3.44 (m, 1H), 3.08-3.21 (m, 4H), 2.19-2.33 (m, 2H), 1.85 (qd, J=12.22, 3.88 Hz, 2H), 1.37 (t, J=7.25 Hz, 3H). ¹⁹F NMR (376 MHz, Methanol-d₄) δ ppm −133.250 (s, 1F), −215.198 (s, 1F).

### Example 69: Synthesis of Compound 205

### Synthesis of B40

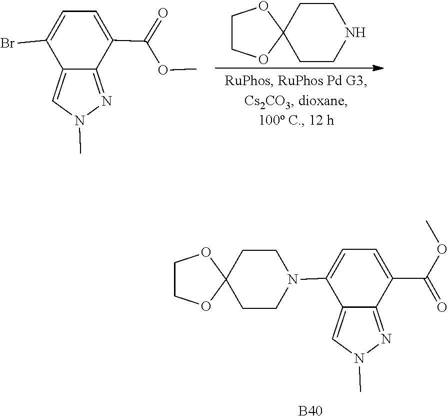

To a suspension of methyl 4-bromo-2-methyl-indazole-7-carboxylate (5 g, 18.5 mmol), 1,4-dioxa-8-azaspiro[4.5]decane (2.66 g, 18.5 mmol, 2.4 mL) in dioxane (71.7 mL) was added Cs₂CO₃ (18.1 g, 55.7 mmol), RuPhos (1.99 g, 4.27 mmol) and RuPhos Pd G3 (2.02 g, 2.42 mmol) successively, and the reaction was stirred at 100° C. for 12 h. The reaction mixture was diluted with EtOAc (100 mL) and filtered. The filtrate was concentrated under reduced pressure and purified by silica gel column chromatography (10% MeOH in EtOAc) to afford methyl 2-methyl-4-(1,4-dioxa-8-azaspiro[4.5]decan-8-yl)-2H-indazole-7-carboxylate (B40, 5 g, 79% yield) as a solid. LCMS (ESI, m/z): 332.2 [M+H]⁺. ¹H NMR (400 MHz, DMSO-d₆) δ ppm 8.59 (s, 1H), 7.84 (d, J=8.13 Hz, 1H), 6.39 (d, J=8.13 Hz, 1H), 4.15 (s, 3H), 3.94 (s, 4H), 3.80 (s, 3H), 3.43-3.53 (m, 4H), 1.77-1.86 (m, 4H).

### Synthesis of B41

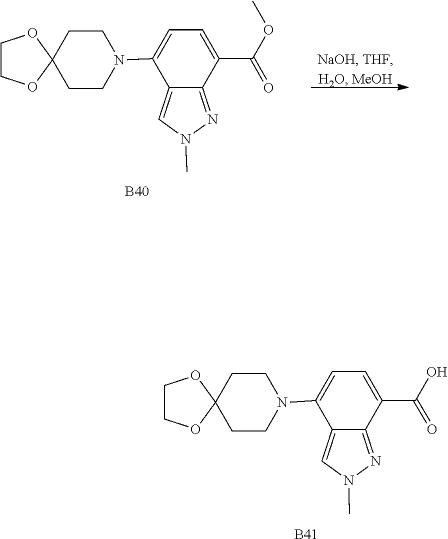

To a solution of methyl 4-(1,4-dioxa-8-azaspiro[4.5]decan-8-yl)-2-methyl-indazole-7-carboxylate (B40, 5 g, 15 mmol) in tetrahydrofuran (18 mL) and methanol (5 mL) was added a solution of NaOH (3.02 g, 75.4 mmol, 1.4 mL) in H₂O (18 mL), and the reaction was stirred at 50° C. for 12 h. The reaction mixture was concentrated under reduced pressure to give a residue. The residue was dissolved with water (100 mL), extracted with EtOAc (50 mL). The aqueous phase was adjusted pH=2 by addition of aqueous HCl (12 M, 10 mL), extracted with EtOAc (3×100 mL), dried over Na₂SO₄ and filtered. The organic phase was concentrated under reduced pressure to afford 2-methyl-4-(1,4-dioxa-8-azaspiro[4.5]decan-8-yl)-2H-indazole-7-carboxylic acid (B41, 4.0 g, 84% yield). LCMS (ESI, m/z): 318.1 [M+H]⁺. ¹H NMR (400 MHz, DMSO-d₆) δ ppm 11.93 (s, 1H), 8.65 (s, 1H), 7.82 (d, J=8.03 Hz, 1H), 6.42 (d, J=8.16 Hz, 1H), 4.16 (s, 3H), 3.94 (s, 4H), 3.46-3.55 (m, 4H), 1.76-1.86 (m, 4H).

### Synthesis of B42

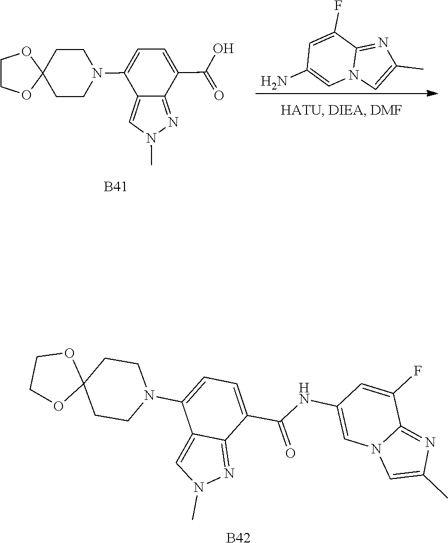

To a solution of 4-(1,4-dioxa-8-azaspiro[4.5]decan-8-yl)-2-methyl-indazole-7-carboxylic acid (B41, 1.5 g, 4.7 mmol) and o-(7-azabenzotriazol-1-yl)-n,n,n,n-tetramethyluronium hexafluorophosphate (2.2 g, 5.7 mmol) in N,N-dimethyl formamide (21 mL) was added N,N-diethylpropan-2-amine (1.53 g, 11.8 mmol, 2.1 mL), and the reaction was stirred at rt for 30 mins. Then, a mixture of 8-fluoro-2-methyl-imidazo[1,2-a]pyridin-6-amine (1.01 g, 6.14 mmol) and N,N-diethylpropan-2-amine (1.53 g, 11.8 mmol, 2.1 mL) in N,N-dimethylformamide (21 mL) was added to the reaction mixture. The reaction was stirred at 50° C. for 12 h. The reaction mixture was diluted with EtOAc (50 mL), washed with brine (3×50 mL), dried over Na₂SO₄, and filtered. The filtrate was concentrated under reduced pressure and purified by silica gel column chromatography (0-18% EtOAc in PE) to afford 4-(1,4-dioxa-8-azaspiro[4.5]decan-8-yl)-N-(8-fluoro-2-methyl-imidazo[1,2-a]pyridin-6-yl)-2-methyl-indazole-7-carboxamide (B42, 930 mg, 40% yield) as a solid. LCMS (ESI, m/z): 465.3 [M+H]⁺. ¹H NMR (400 MHz, DMSO-d₆) δ ppm 11.05 (s, 1H), 9.21 (s, 1H), 8.78 (s, 1H), 7.82-8.03 (m, 2H), 7.33 (br d, J=12.26 Hz, 1H), 6.52 (br d, J=8.13 Hz, 1H), 4.29 (s, 3H), 3.92-3.98 (m, 4H), 3.52 (br s, 4H), 2.35 (s, 3H), 1.83 (br s, 4H).

### Synthesis of B43

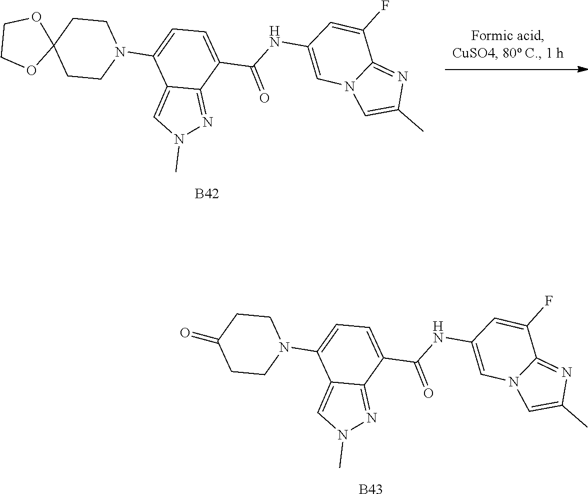

To a solution of 4-(1,4-dioxa-8-azaspiro[4.5]decan-8-yl)-N-(8-fluoro-2-methyl-imidazo-[1,2-a]pyridin-6-yl)-2-methyl-indazole-7-carboxamide (200 mg, 430 μmol) in formic acid (4 mL) was added CuSO₄ (2.06 mg, 12.9 μmol) at 0° C. under N₂ protection. The reaction mixture was stirred at 80° C. for 1 h. The reaction mixture was concentrated under reduced pressure to get an oil. The oil was triturated with MeCN (40 mL) at 25° C. for 30 mins. Then, the mixture was filtered and the filtrate was concentrated under reduced pressure to get N-(8-fluoro-2-methylimidazo[1,2-a]pyridin-6-yl)-2-methyl-4-(4-oxopiperidin-1-yl)-2H-indazole-7-carboxamide (B43, 75 mg, 33% yield) as a solid. LCMS (ESI, m/z) 421.3 [M+H]⁺. ¹H NMR (400 MHz, DMSO-d₆) δ ppm 12.45-13.13 (m, 1H), 11.09 (s, 1H), 9.25 (s, 1H), 8.87 (s, 1H), 8.13 (s, 1H), 7.99 (d, J=8.13 Hz, 1H), 7.93 (d, J=2.25 Hz, 1H), 6.51 (d, J=8.25 Hz, 1H), 4.30 (s, 3H), 3.88 (t, J=6.13 Hz, 4H), 2.62 (t, J=6.07 Hz, 4H), 2.36 (s, 3H).

### Synthesis of Compound 205

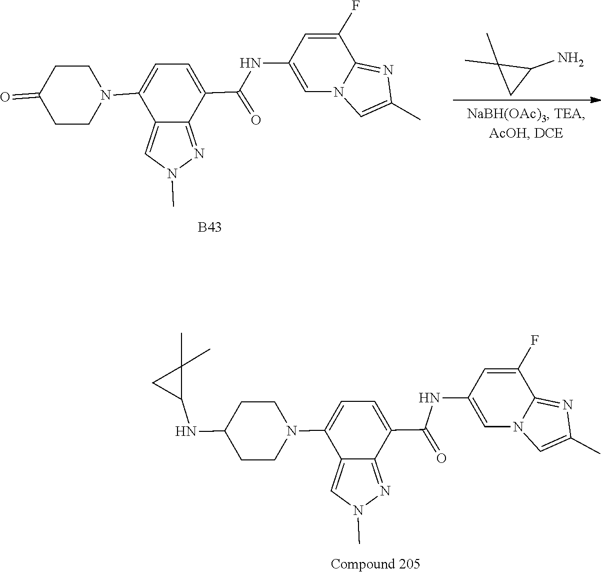

To a suspension of 2,2-dimethylcyclopropanamine (23 mg, 190 μmol) in 1,2-dichloroethane (768 μL) was added triethylamine (38.5 mg, 380 μmol), and the reaction was stirred at 25° C. for 20 mins. N-(8-fluoro-2-methyl-imidazo[1,2-a]pyridin-6-yl)-2-methyl-4-(4-oxo-1-piperidyl)indazole-7-carboxamide (80 mg, 190 μmol), acetic acid (23 mg, 380 μmol) and NaBH(OAc)₃ (60.4 mg, 285 μmol) were then added to the reaction mixture successively. The reaction was stirred at 25° C. for 12 h. The reaction mixture was quenched with water (1 mL), extracted with DCM (3×5 mL), dried over Na₂SO₄, and filtered. The filtrate was concentrated under reduced pressure and purified by prep-HPLC (Condition 9, Gradient 5) to afford 4-(4-((2,2-dimethylcyclopropyl)amino)piperidin-1-yl)-N-(8-fluoro-2-methylimidazo[1,2-a]pyridin-6-yl)-2-methyl-2H-indazole-7-carboxamide (Compound 205, 10.1 mg, 11% yield). LCMS (ESI, m/z): 490.3 [M+H]⁺. ¹H NMR (400 MHz, Methanol-d₄) δ ppm 9.53 (s, 1H), 8.66 (s, 1H), 8.06-8.18 (m, 3H), 6.65 (d, J=8.25 Hz, 1H), 4.37 (s, 3H), 4.14-4.23 (m, 2H), 3.57 (tt, J=11.83, 4.05 Hz, 1H), 3.11-3.21 (m, 2H), 2.55-2.61 (m, 4H), 2.40-2.48 (m, 1H), 2.26-2.34 (m, 1H), 1.89-2.02 (m, 2H), 1.34 (s, 3H), 1.19 (s, 3H), 1.00 (t, J=7.00 Hz, 1H), 0.80 (dd, J=6.19, 4.32 Hz, 1H).

An analogous method was followed to obtain the following compounds.

| | Starting | |
|---|---|---|
| Compound | Material | Characterization |
| 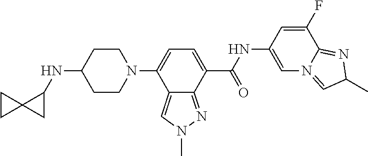 Compound 203 | 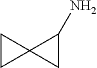 | The last step was ran in DCM. The residue was purified by prep-HPLC (Condition 9, Gradient 2) to get N-(8- fluoro-2-methyl-imidazo[1,2-a]-pyridin- 6-yl)-2-methyl-4-[4-(spiro[2.2]pentan-2- ylamino)-1-piperidyl] indazole-7- carboxamide (Compound 203, 7.4 mg, 43% yield) as a solid. LCMS (ESI, m/z): 488.3 [M + H]⁺. ¹H NMR (400 MHz, Methanol-d4) δ ppm 9.56 (s, 1H), 8.61 (s, 1H), 8.04-8.19 (m, 3H), 6.62 (d, J = 8.25 Hz, 1H), 4.36 (s, 3H), 4.16 (br d, J = 12.13 Hz, 2H), 3.48-3.58 (m, 1H), 3.13 (br s, 3H), 2.59 (s, 3H), 2.33-2.44 (m, 1H), 2.20-2.28 (m, 1H), 1.83-1.96 (m, 2H), 1.46-1.52 (m, 1H), 1.30 (dd, J = 6.13, 3.38 Hz, 2H), 1.07-1.17 (m, 3H), 1.00- 1.06 (m, 1H). |
| 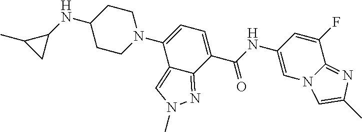 Compound 204 | 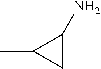 | The oil was purified by prep-HPLC to afford N-(8-fluoro-2-methyl- imidazo[1,2-a]pyridin-6-yl)-2-methyl-4- [4-[(2-methyl-cyclopropyl)amino]-1- piperidyl] indazole-7-carboxamide (Compound 204, 13.2 mg, 23% yield) as a solid. LCMS (ESI, m/z): 476.2 [M + H]⁺. ¹H NMR (400 MHz, Methanol- d4) δ ppm 9.56 (s, 1H), 8.59 (s, 1H), 8.01- 8.24 (m, 3H), 6.62 (d, J = 8.13 Hz, 1H), 4.36 (s, 3H), 4.10-4.21 (m, 2H), 3.46- 3.55 (m, 1H), 3.09-3.18 (m, 2H), 2.59 (s, 3H), 2.55 (dt, J = 7.29, 3.67 Hz, 1H), 2.23-2.41 (m, 2H), 1.76-1.99 (m, 2H), 1.29 (br d, J = 2.75 Hz, 1H), 1.17 (d, J = 6.00 Hz, 3H), 1.04 (ddd, J = 9.85, 6.28, 3.75 Hz, 1H), 0.80 (q, J = 6.80 Hz, 1H). |

### Example 70: Synthesis of Compounds 496-502, 505, 525-536, 549, 557, 562-564, 575-576, 601, and 604

### General Procedure A

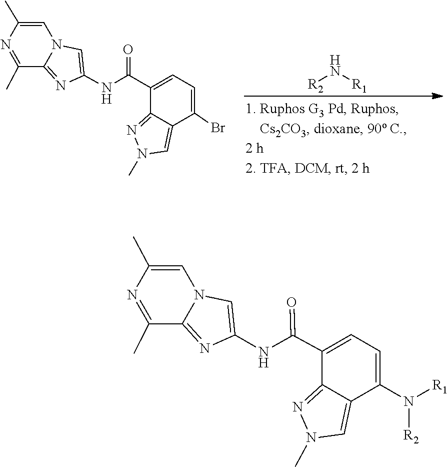

To a mixture of 4-bromo-N-(6,8-dimethylimidazo[1,2-a]pyrazin-2-yl)-2-methyl-indazole-7-carboxamide (50.0 mg, 125.6 μmol, 1.0 eq) and amine (150.8 μmol, 1.2 eq) in dioxane (1 mL) were added Cs₂CO₃ (81.6 mg, 251.2 μmol, 2.0 eq) and RuPhos (11.7 mg, 25.1 μmol, 0.2 eq) and RuPhos Pd G3 (10.5 mg, 12.6 μmol, 0.1 eq), and the reaction was stirred for 2 h at 90° C. under N₂. The resulting mixture was concentrated under reduced pressure and purified by silica gel column chromatography (9% MeOH in DCM) to afford the product. The product was dissolved in DCM (1.5 mL). To this solution was added TFA (0.5 mL), and the reaction was stirred for 2 h at room temperature. The resulting mixture was concentrated under reduced pressure and adjusted to pH 8 with saturated NaHCO₃ (aq.). The residue was purified by reversed-phase flash chromatography to give the final product.

An analogous method was followed to obtain the following compounds.

| Compound | Characterization |
|---|---|
| 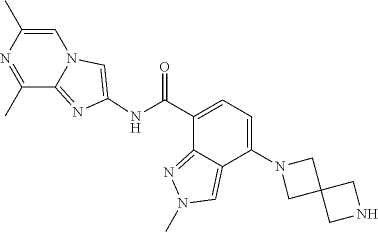 [SMILES: Cc1cn2cc(NC(=O)c3ccc(N4CC5(CNC5)C4)c4cn(C)nc34)nc2c(C)n1] Compound 496 | The compound was obtained as a solid (Compound 496, 14 mg, 34% yield) following the general procedure. LCMS (ES, m/z): 417 [M + H]⁺. ¹H NMR (400 MHz, DMSO-d₆) δ 11.30 (s, 1H), 8.68 (s, 1H), 8.30 (d, J = 2.8 Hz, 2H), 7.98 (d, J = 8.1 Hz, 1H), 5.91 (d, J = 8.1 Hz, 1H), 4.33 (s, 4H), 4.28 (s, 3H), 4.06 (s, 1H), 3.68 (s, 4H), 2.69 (s, 3H), 2.37 (d, J = 0.9 Hz, 3H). |
| 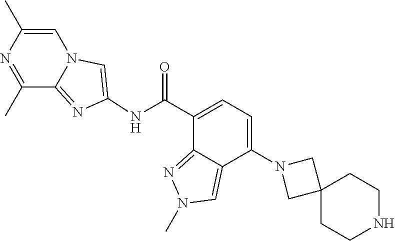 [SMILES: Cc1cn2cc(NC(=O)c3ccc(N4CC5(CCNCC5)C4)c4cn(C)nc34)nc2c(C)n1] Compound 562 | The compound was obtained as a solid (Compound 562, 18 mg, 25% yield) following the general procedure. LCMS (ES, m/z): 445 [M + H]⁺. ¹H NMR (300 MHz, DMSO-d₆) δ 11.36 (s, 1H), 8.63 (s, 1H), 8.55-8.52 (m, 2H), 8.40 (d, J = 5.1 Hz, 2H), 7.99 (d, J = 8.1 Hz, 1H), 5.90 (d, J = 8.2 Hz, 1H), 4.27 (s, 3H), 4.06 (s, 3H), 3.09 (s, 4H), 2.73 (s, 3H), 2.39 (s, 3H), 1.99 (t, J = 5.6 Hz, 4H). |
| 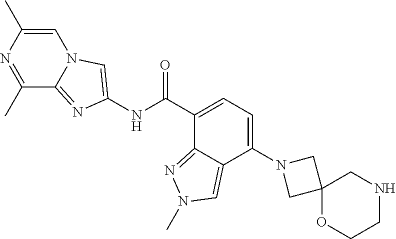 [SMILES: Cc1cn2cc(NC(=O)c3ccc(N4CC5(CNCCO5)C4)c4cn(C)nc34)nc2c(C)n1] Compound 497 | The compound was obtained as a solid (Compound 497, 6 mg, 15% yield) following the general procedure. LCMS (ES, m/z): 447 [M + H]⁺. ¹H NMR (400 MHz, DMSO-d₆) δ 11.28 (s, 1H), 8.70 (s, 1H), 8.29 (d, J = 2.5 Hz, 2H), 7.97 (d, J = 8.1 Hz, 1H), 5.94 (d, J = 8.2 Hz, 1H), 4.26 (d, J = 2.4 Hz, 3H), 4.17 (d, J = 8.8 Hz, 2H), 4.03 (d, J = 8.3 Hz, 2H), 3.60 (t, J = 4.7 Hz, 2H), 2.89 (s, 2H), 2.67-2.65 (m, 5H), 2.35 (s, 3H). |
| 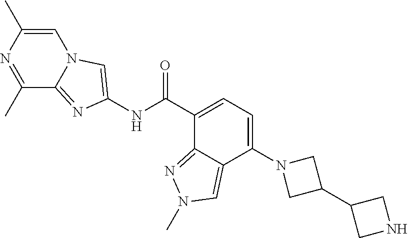 [SMILES: Cc1cn2cc(NC(=O)c3ccc(N4CC(C5CNC5)C4)c4cn(C)nc34)nc2c(C)n1] Compound 498 | The compound was obtained as a solid (Compound 498, 15 mg, 27% yield) following the general procedure. LCMS (ES, m/z): 431 [M + H]⁺. ¹H NMR (400 MHz, DMSO-d₆) δ 11.27 (s, 1H), 8.66 (s, 1H), 8.28 (d, J = 2.2 Hz, 2H), 7.96 (d, J = 8.1 Hz, 1H), 5.87 (d, J = 8.2 Hz, 1H), 4.33 (t, J = 8.2 Hz, 2H), 4.25 (s, 3H), 3.94-3.89 (m, 2H), 3.65-3.62 (m, 2H), 3.30-3.20 (m, 2H), 3.17-3.10 (m, 1H), 3.02-2.88 (m, 1H), 2.67 (s, 3H), 2.35 (s, 3H). |
| 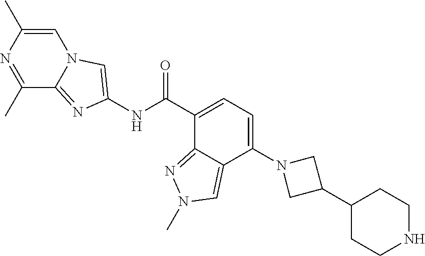 [SMILES: Cc1cn2cc(NC(=O)c3ccc(N4CC(C5CCNCC5)C4)c4cn(C)nc34)nc2c(C)n1] Compound 499 | The compound was obtained as a solid (Compound 499, 19 mg, 31% yield) following the general procedures. LCMS (ES, m/z): 459 [M + H]⁺. ¹H NMR (400 MHz, DMSO-d₆) δ 11.29 (s, 1H), 8.69 (s, 1H), 8.30 (s, 2H), 7.97 (d, J = 8.0 Hz, 1H), 5.88 (d, J = 8.1 Hz, 1H), 4.32-4.25 (m, 5H), 4.02-3.97 (m, 2H), 2.96 (d, J = 11.9 Hz, 2H), 2.69 (s, 3H), 2.57 (s, 1H), 2.44 (d, J = 12.5 Hz, 2H), 2.37 (s, 3H), 1.71-1.52 (m, 3H), 1.22-0.77 (m, 2H). |
| 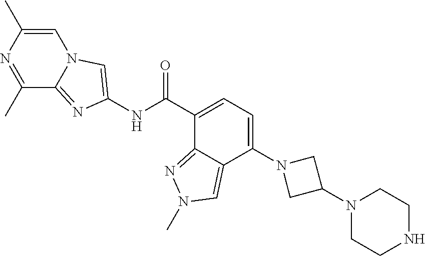 [SMILES: Cc1cn2cc(NC(=O)c3ccc(N4CC(N5CCNCC5)C4)c4cn(C)nc34)nc2c(C)n1] Compound 500 | The compound was obtained as a solid (Compound 500, 13 mg, 26% yield) following the general procedure. LCMS (ES, m/z): 460 [M + H]⁺. ¹H NMR (400 MHz, DMSO-d₆) δ 11.27 (s, 1H), 8.67 (s, 1H), 8.28 (d, J = 2.4 Hz, 2H), 7.96 (d, J = 8.0 Hz, 1H), 5.89 (d, J = 8.2 Hz, 1H), 4.32-4.25 (m, 5H), 4.04 (t, J = 6.9 Hz, 2H), 3.49- 3.48 (m, 1H), 2.72 (t, J = 4.6 Hz, 4H), 2.67 (s, 3H), 2.35 (s, 3H), 2.31-2.15 (m, 4H). |
| 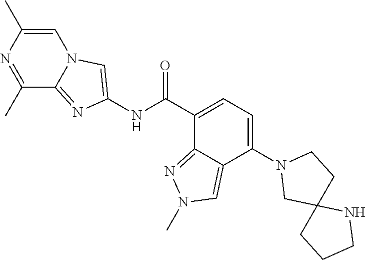 [SMILES: Cc1cn2cc(NC(=O)c3ccc(N4CCC5(CCCN5)C4)c4cn(C)nc34)nc2c(C)n1] Compound 501 | The compound was obtained as a solid (Compound 501, 16 mg, 32% yield) following the general procedure. LCMS (ES, m/z): 445 [M + H]⁺. ¹H NMR (400 MHz, DMSO-d₆) δ 11.36 (s, 1H), 8.84 (s, 1H), 8.30 (d, J = 2.4 Hz, 2H), 7.97 (d, J = 8.3 Hz, 1H), 6.01 (d, J = 8.4 Hz, 1H), 4.27 (s, 3H), 3.71 (d, J = 7.6 Hz, 2H), 3.57 (q, J = 10.2 Hz, 2H), 2.89 (d, J = 5.1 Hz, 2H), 2.69 (s, 3H), 2.37 (s, 3H), 2.00 (t, J = 6.9 Hz, 2H), 1.77 (td, J = 12.3, 10.7, 6.2 Hz, 4H). |
| 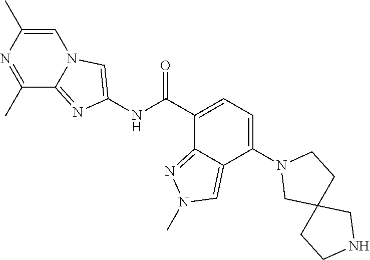 [SMILES: Cc1cn2cc(NC(=O)c3ccc(N4CCC5(CCNC5)C4)c4cn(C)nc34)nc2c(C)n1] Compound 525 | The compound was obtained as a solid (Compound 525, 17 mg, 40% yield) following the general procedure. LCMS (ES, m/z): 445 [M + H]⁺. ¹H NMR (400 MHz, DMSO-d₆) δ 11.36 (s, 1H), 8.84 (d, J = 6.4 Hz, 1H), 8.29 (d, J = 2.3 Hz, 2H), 7.97 (d, J = 8.2 Hz, 1H), 6.04 (t, J = 8.0 Hz, 1H), 4.27 (s, 3H), 3.70-3.65 (m, 2H), 3.60 (q, J = 10.2, 9.5 Hz, 2H), 3.28-3.25 (m, 1H), 2.94-2.75 (m, 2H), 2.73-2.70 (m, 1H), 2.69 (s, 3H), 2.36 (s, 3H), 2.11-1.95 (m, 2H), 1.80-1.65 (m, 2H). |
| 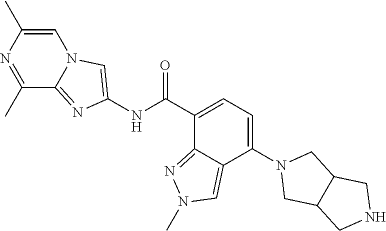 [SMILES: Cc1cn2cc(NC(=O)c3ccc(N4CC5CNCC5C4)c4cn(C)nc34)nc2c(C)n1] Compound 526 | The compound was obtained as a solid (Compound 526, 13 mg, 28% yield) following the general procedure. LCMS (ES, m/z): 431 [M + H]⁺. ¹H NMR (400 MHz, DMSO-d₆) δ 11.38 (s, 1H), 8.86 (s, 1H), 8.29 (s, 2H), 7.97 (d, J = 8.3 Hz, 1H), 6.05 (d, J = 8.4 Hz, 1H), 4.27 (s, 3H), 3.94-3.88 (m, 2H), 3.54-3.44 (m, 2H), 3.10-2.88 (m, 4H), 2.82-2.73 (m, 2H), 2.69 (s, 3H), 2.36 (s, 3H). |
| 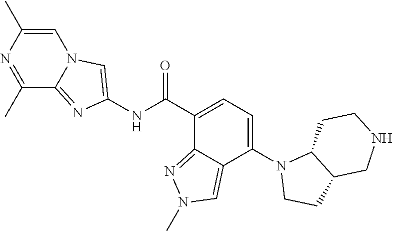 [SMILES: Cc1cn2cc(NC(=O)c3ccc(N4CC[C@H]5CNCC[C@H]54)c4cn(C)nc34)nc2c(C)n1] Compound 502 | The compound was obtained as a solid (Compound 502, 13 mg, 31% yield) following the general procedure. LCMS (ES, m/z): 445 [M + H]⁺. ¹H NMR (400 MHz, DMSO-d₆) δ 11.37 (s, 1H), 8.81 (s, 1H), 8.30 (d, J = 4.0 Hz, 2H), 7.95 (d, J = 8.4 Hz, 1H), 6.12 (d, J = 8.5 Hz, 1H), 4.27 (s, 3H), 4.20-4.15 (m, 1H), 3.87-3.61 (m, 2H), 3.02-2.92 (m, 2H), 2.90-2.82 (m, 1H), 2.72-2.65 (m, 3H), 2.50-2.20 (m, 6H), 2.10-1.86 (m, 2H), 1.36-1.21 (m, 1H). |
| 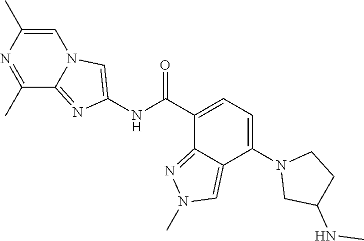 [SMILES: CNC1CCN(c2ccc(C(=O)Nc3cn4cc(C)nc(C)c4n3)c3nn(C)cc23)C1] Compound 505 | The compound was obtained as a solid (Compound 505, 13 mg, 24% yield) following the general procedure. LCMS (ES, m/z): 419 [M + H]⁺. ¹H NMR (400 MHz, DMSO-d₆) δ 11.37 (d, J = 2.0 Hz, 1H), 8.85 (s, 1H), 8.30 (d, J = 3.0 Hz, 2H), 7.98 (d, J = 7.9 Hz, 1H), 6.03 (d, J = 8.7 Hz, 1H), 4.28 (d, J = 2.1 Hz, 3H), 3.78-3.66 (m, 4H), 2.69 (d, J = 2.1 Hz, 3H), 2.38-2.34 (m, 6H), 2.15-2.10 (m, 1H), 1.93-1.90 (m, 1H), 1.24-1.23 (m, 1H), 0.85 (d, J = 7.2 Hz, 1H). |
| 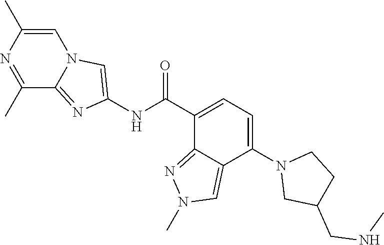 [SMILES: CNCC1CCN(c2ccc(C(=O)Nc3cn4cc(C)nc(C)c4n3)c3nn(C)cc23)C1] Compound 527 | The compound was obtained as a solid (Compound 527, 18 mg, 33% yield) following the general procedure. LCMS (ES, m/z): 433 [M + H]⁺. ¹H NMR (400 MHz, DMSO-d₆) δ 11.34 (s, 1H), 8.82 (s, 1H), 8.28 (d, J = 2.3 Hz, 2H), 7.95 (d, J = 8.3 Hz, 1H), 6.01 (d, J = 8.4 Hz, 1H), 4.26 (s, 3H), 3.80-3.61 (m, 3H), 3.45-3.34 (m, 1H), 2.67 (s, 3H), 2.55 (s, 2H), 2.34 (s, 3H), 2.30 (s, 3H), 2.14- 2.10 (m, 2H), 1.82-1.75 (m, 1H). |
| 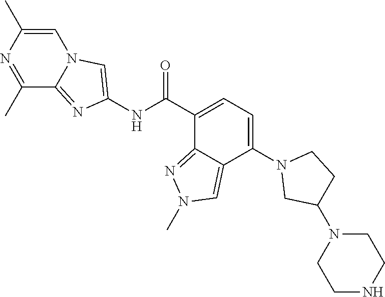 [SMILES: Cc1cn2cc(NC(=O)c3ccc(N4CCC(N5CCNCC5)C4)c4cn(C)nc34)nc2c(C)n1] Compound 528 | The compound was obtained as a solid (Compound 528, 18 mg, 52% yield) following the general procedure. LCMS (ES, m/z): 474 [M + H]⁺. ¹H NMR (400 MHz, DMSO-d₆) δ 11.37 (s, 1H), 8.88 (s, 1H), 8.30 (d, J = 2.6 Hz, 2H), 7.98 (d, J = 8.2 Hz, 1H), 6.06 (d, J = 8.4 Hz, 1H), 4.28 (s, 3H), 3.93-2.86 (m, 1H), 3.80-3.74 (m, 1H), 3.70-3.57 (m, 1H), 3.49-3.40 (m, 1H), 3.02-2.94 (m, 1H), 2.77 (s, 4H), 2.69 (s, 3H), 2.50-2.47 (m, 4H), 2.37 (s, 3H), 2.70-2.27 (s, 1H), 1.88 (t, J = 10.3 Hz, 1H). |
| 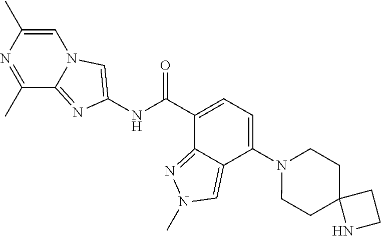 [SMILES: Cc1cn2cc(NC(=O)c3ccc(N4CCC5(CCN5)CC4)c4cn(C)nc34)nc2c(C)n1] Compound 563 | To a solution of tert-butyl 7-[7-[(6,8- dimethylimidazo[1,2-a]pyrazin-2-yl)carbamoyl]-2- methyl-indazol-4-yl]-1,7-diazaspiro[3.5]nonane-1- carboxylate (0.06 g, 110.2 μmol) in DCM (1 mL) was added TMSOTf (122.4 mg, 550.8 μmol, 99.69 μL), and the reaction was stirred for 4 h at room temperature. The resulting mixture was concentrated under reduced pressure, adjusted to pH 8 with saturated NaHCO3 (aq.), and purified by reversed-phase flash chromatography (Condition 3, Gradient 12) to afford 4-(1,7- diazaspiro[3.5]nonan-7-yl)-N-(6,8-dimethyl- imidazo[1,2-a]pyrazin-2-yl)-2-methyl-indazole-7- carboxamide (Compound 563, 8.1 mg, 18.2 μmol, 17% yield) as a solid. LCMS (ES, m/z): 445 [M + H]⁺. 1H NMR (400 MHz, Methanol-d₄) δ 8.50 (s, 1H), 8.31 (s, 1H), 8.17 (s, 1H), 8.10 (d, J = 8.1 Hz, 1H), 6.55 (d, J = 8.2 Hz, 1H), 4.37 (s, 3H), 3.59 (q, J = 7.8 Hz, 4H), 3.43- 3.35 (m, 2H), 2.79 (s, 3H), 2.45 (s, 3H), 2.29 (t, J = 7.8 Hz, 2H), 2.06 (ddt, J = 17.4, 13.0, 9.3 Hz, 4H). |
| 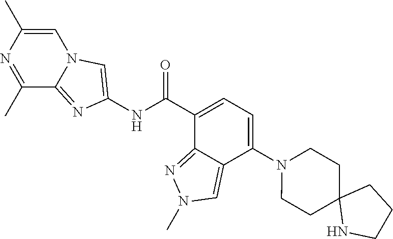 [SMILES: Cc1cn2cc(NC(=O)c3ccc(N4CCC5(CCCN5)CC4)c4cn(C)nc34)nc2c(C)n1] Compound 529 | The compound was obtained as a solid (Compound 529, 18 mg, 26% yield) following the general procedure. LCMS (ES, m/z): 459 [M + H]⁺. ¹H NMR (400 MHz, DMSO-d₆) δ 11.44 (s, 1H), 8.78 (s, 1H), 8.32 (d, J = 3.5 Hz, 2H), 8.02 (d, J = 8.1 Hz, 1H), 6.50 (d, J = 8.3 Hz, 1H), 4.30 (s, 3H), 3.50 (ddt, J = 23.3, 12.5, 6.8 Hz, 4H), 2.86 (t, J = 6.8 Hz, 2H), 2.70 (s, 3H), 2.35 (s, 3H), 2.21- 1.80 (m, 1H), 1.78-1.58 (m, 6H), 1.55 (dd, J = 8.7, 6.4 Hz, 2H). |
| 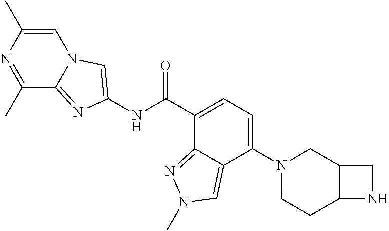 [SMILES: Cc1cn2cc(NC(=O)c3ccc(N4CCC5NCC5C4)c4cn(C)nc34)nc2c(C)n1] Compound 530 | The compound was obtained as a solid (Compound 530, 14 mg, 32% yield) following the general procedure. LCMS (ES, m/z): 431 [M + H]⁺. ¹H NMR (400 MHz, DMSO-d₆) δ 11.43 (d, J = 3.2 Hz, 1H), 8.86 (s, 1H), 8.31 (s, 2H), 8.01 (d, J = 8.3 Hz, 1H), 6.31 (t, J = 8.7 Hz, 1H), 4.30 (s, 3H), 4.19-4.01 (m, 2H), 4.00-3.50 (m, 5H), 3.18 (t, J = 6.7 Hz, 1H), 3.03 (d, J = 5.1 Hz, 1H), 2.70 (s, 3H), 2.37 (s, 3H), 2.26-1.55 (m, 2H). |
| 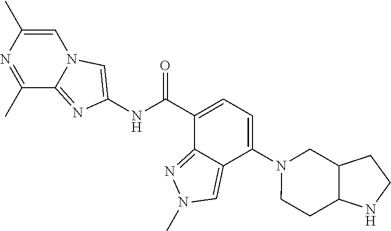 [SMILES: Cc1cn2cc(NC(=O)c3ccc(N4CCC5NCCC5C4)c4cn(C)nc34)nc2c(C)n1] Compound 531 | The compound was obtained as a solid (Compound 531, 17 mg, 44% yield) following the general procedure. LCMS (ES, m/z): 445 [M + H]⁺. ¹H NMR (400 MHz, DMSO-d₆) δ 11.42 (s, 1H), 8.81 (s, 1H), 8.31 (s, 2H), 8.00 (d, J = 8.3 Hz, 1H), 6.36 (d, J = 8.4 Hz, 1H), 4.29 (s, 3H), 3.68 (dd, J = 12.7, 5.3 Hz, 1H), 3.56-3.51 (m, 2H), 3.29- 3.25 (m, 1H), 3.25-3.17 (m, 1H), 3.01-2.90 (m, 1H), 2.75-2.70 (m, 4H), 2.40-2.30 (m, 4H), 2.08-1.94 (m, 1H), 1.85 (dt, J = 8.1, 4.3 Hz, 1H), 1.76 (d, J = 6.4 Hz, 1H), 1.54-1.33 (m, 1H). |
| 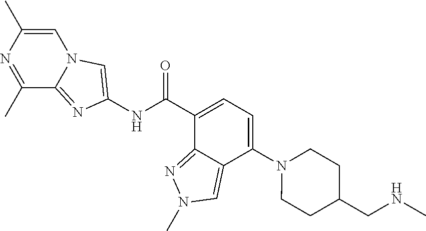 [SMILES: CNCC1CCN(c2ccc(C(=O)Nc3cn4cc(C)nc(C)c4n3)c3nn(C)cc23)CC1] Compound 532 | The compound was obtained as a solid (Compound 532, 14 mg, 26% yield) following the general procedure. LCMS (ES, m/z): 447[M + H]⁺. ¹H NMR (300 MHz, DMSO-d₆) δ 11.44 (s, 1H), 8.78 (s, 1H), 8.32 (d, J = 2.4 Hz, 2H), 8.03 (d, J = 8.1 Hz, 1H), 6.50 (d, J = 8.2 Hz, 1H), 4.30 (s, 3H), 3.97 (d, J = 12.6 Hz, 2H), 2.98 (t, J = 12.1 Hz, 2H), 2.70 (s, 3H), 2.45 (d, J = 6.5 Hz, 2H), 2.37 (s, 3H), 2.31 (s, 3H), 1.87 (d, J = 12.6 Hz, 2H), 1.75-1.71 (m, 1H), 1.40-1.25 (m, 2H). |
| 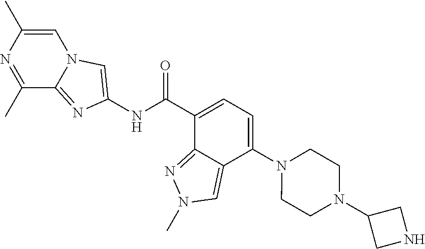 [SMILES: Cc1cn2cc(NC(=O)c3ccc(N4CCN(C5CNC5)CC4)c4cn(C)nc34)nc2c(C)n1] Compound 533 | The compound was obtained as a solid (Compound 533, 14 mg, 32% yield) following the general procedure. LCMS (ES, m/z): 460 [M + H]⁺. ¹H NMR (400 MHz, DMSO-d₆) δ 11.46 (s, 1H), 8.82 (s, 1H), 8.32 (d, J = 3.0 Hz, 2H), 8.04 (d, J = 8.1 Hz, 1H), 6.53 (d, J = 8.2 Hz, 1H), 4.30 (s, 3H), 3.50-3.40 (m, 6H), 3.40-3.38 (m, 2H), 3.18 (t, J = 6.8 Hz, 1H), 2.70 (s, 3H), 2.47 (t, J = 4.9 Hz, 4H), 2.38 (s, 3H). |
| 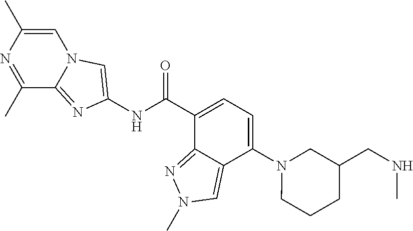 [SMILES: CNCC1CCCN(c2ccc(C(=O)Nc3cn4cc(C)nc(C)c4n3)c3nn(C)cc23)C1] Compound 534 | The compound was obtained as a solid (Compound 534, 14 mg, 27% yield) following the general procedure. LCMS (ES, m/z): 447 [M + H]⁺. ¹H NMR (400 MHz, DMSO-d₆) δ 11.44 (s, 1H), 8.83 (s, 1H), 8.32 (d, J = 2.1 Hz, 2H), 8.02 (d, J = 8.1 Hz, 1H), 6.49 (d, J = 8.2 Hz, 1H), 4.30 (s, 3H), 3.83 (t, J = 14.7 Hz, 2H), 3.08 (t, J = 11.2 Hz, 1H), 2.99-2.84 (m, 1H), 2.70 (s, 3H), 2.49-2.38 (m, 2H), 2.37 (s, 3H), 2.32 (s, 3H), 1.94-1.61 (m, 4H), 1.32-1.15 (m, 1H). |
| 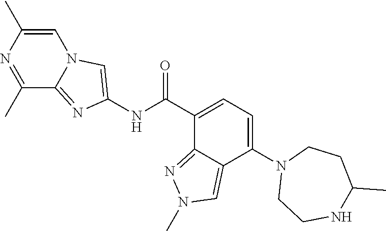 [SMILES: Cc1cn2cc(NC(=O)c3ccc(N4CCNC(C)CC4)c4cn(C)nc34)nc2c(C)n1] Compound 535 | The compound was obtained as a solid (Compound 535, 8.4 mg, 25% yield) following the general procedure. LCMS (ES, m/z): 433 [M + H]⁺. ¹H NMR (300 MHz, DMSO-d₆) δ 11.42 (s, 1H), 8.73 (s, 1H), 8.30 (s, 2H), 7.98 (d, J = 8.4 Hz, 1H), 6.24 (d, J = 8.5 Hz, 1H), 4.29 (s, 3H), 3.90-3.72 (m, 3H), 3.65-3.58 (m, 1H), 3.18 (d, J = 14.0 Hz, 1H), 2.95 (t, J = 12.1 Hz, 1H), 2.70 (s, 4H), 2.37 (s, 3H), 1.99 (d, J = 14.2 Hz, 1H), 1.60-1.46 (m, 1H), 1.04 (d, J = 6.2 Hz, 3H). |
| 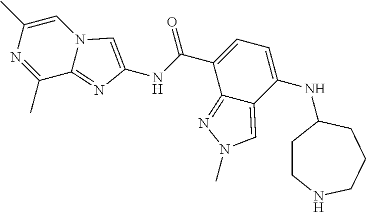 [SMILES: Cc1cn2cc(NC(=O)c3ccc(NC4CCCNCC4)c4cn(C)nc34)nc2c(C)n1] Compound 536 | The compound was obtained as a solid (Compound 536, 14 mg, 36% yield) following the general procedure. LCMS (ES, m/z): 433 [M + H]⁺. ¹H NMR (400 MHz, DMSO-d₆) δ 11.23 (s, 1H), 8.68 (d, J = 8.5 Hz, 1H), 8.30 (d, J = 3.3 Hz, 2H), 7.98 (d, J = 8.1 Hz, 1H), 6.94 (d, J = 7.8 Hz, 1H), 6.09 (d, J = 8.4 Hz, 1H), 4.27 (s, 3H), 3.82- 3.78 (m, 1H), 2.95-2.75 (m, 3H), 2.69 (s, 3H), 2.37 (s, 3H), 2.15-1.86 (m, 2H), 1.80-1.50 (m, 4H). |
| 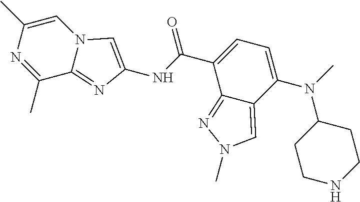 [SMILES: Cc1cn2cc(NC(=O)c3ccc(N(C)C4CCNCC4)c4cn(C)nc34)nc2c(C)n1] Compound 564 | The compound was obtained as a solid (Compound 564, 7.3 mg, 23% yield) following the general procedure. LCMS (ES, m/z): 433 [M + H]⁺. ¹H NMR (400 MHz, DMSO-d₆) δ 11.74 (s, 1H), 9.20-9.00 (m, 2H), 8.82 (s, 1H), 8.79 (s, 1H), 8.77 (s, 1H), 8.01 (d, J = 8.4 Hz, 1H), 6.43 (d, J = 8.5 Hz, 1H), 4.40-4.29 (m, 4H), 3.36 (d, J = 12.4 Hz, 2H), 3.25-3.11 (m, 2H), 3.06 (s, 3H), 2.94 (s, 3H), 2.52 (s, 3H), 2.18 (qd, J = 12.9, 4.0 Hz, 2H), 1.93- 1.84 (m, 2H). |
| 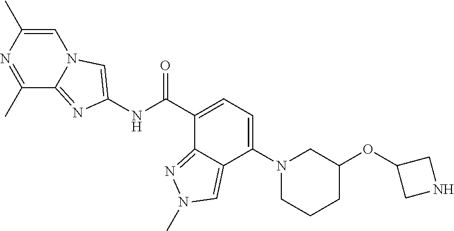 [SMILES: Cc1cn2cc(NC(=O)c3ccc(N4CCCC(OC5CNC5)C4)c4cn(C)nc34)nc2c(C)n1] Compound 601 | The compound was obtained as a solid (Compound 601, 18 mg, 39% yield) following the general procedure. LCMS (ES, m/z): 475 [M + H]⁺. ¹H NMR (400 MHz, DMSO-d₆) δ 11.51 (s, 1H), 8.79 (s, 1H), 8.80-8.60 (m, 2H), 8.42 (d, J = 9.7 Hz, 2H), 8.05 (d, J = 8.1 Hz, 1H), 6.55 (d, J = 8.2 Hz, 1H), 4.59 (p, J = 6.3 Hz, 1H), 4.32 (s, 3H), 4.20-4.05 (m, 2H), 3.84 (dq, J = 11.2, 6.2 Hz, 2H), 3.74 (dd, J = 12.4, 9.1 Hz, 1H), 3.68 (td, J = 7.9, 4.0 Hz, 2H), 3.23 (ddd, J = 12.5, 9.3, 3.0 Hz, 1H), 3.13 (dd, J = 12.2, 7.8 Hz, 1H), 2.75 (s, 3H), 2.41 (s, 3H), 2.00 (d, J = 9.9 Hz, 1H), 1.94-1.86 (m, 1H), 1.66 (dd, J = 9.6, 3.8 Hz, 1H), 1.60-1.48 (m, 1H). |
| 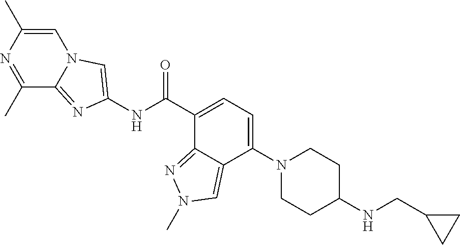 [SMILES: Cc1cn2cc(NC(=O)c3ccc(N4CCC(NCC5CC5)CC4)c4cn(C)nc34)nc2c(C)n1] Compound 575 | The compound was obtained as a solid (Compound 575, 18 mg, 32% yield) following the general procedure. LCMS (ES, m/z): 473 [M + H]⁺. ¹H NMR (400 MHz, DMSO-d₆) δ 8.82 (s, 1H), 8.31 (d, J = 4.7 Hz, 2H), 8.05 (d, J = 8.1 Hz, 1H), 6.56 (d, J = 8.2 Hz, 1H), 4.31 (s, 3H), 4.04 (d, J = 12.8 Hz, 2H), 3.35-3.25 (m, 1H), 3.04 (t, J = 12.3 Hz, 2H), 2.86 (d, J = 7.3 Hz, 2H), 2.70 (s, 3H), 2.37 (s, 3H), 2.17-2.09 (m, 2H), 1.70 (qd, J = 12.1, 3.9 Hz, 2H), 1.03 (ddd, J = 12.5, 7.9, 4.7 Hz, 1H), 0.64-0.54 (m, 2H), 0.39-0.31 (m, 2H). |
| 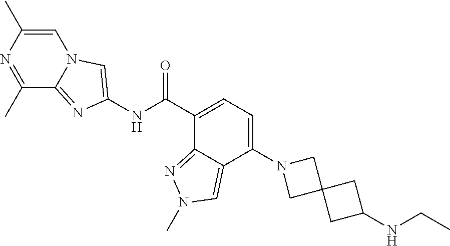 [SMILES: CCNC1CC2(C1)CN(c1ccc(C(=O)Nc3cn4cc(C)nc(C)c4n3)c3nn(C)cc13)C2] Compound 576 | The compound was obtained as a solid (Compound 576, 7.3 mg, 14% yield) following the general procedure. LCMS (ES, m/z): 459 [M + H]⁺. ¹H NMR (400 MHz, DMSO-d₆) δ 11.30 (s, 1H), 8.62 (s, 1H), 8.30 (d, J = 4.3 Hz, 2H), 8.18 (s, 1H), 7.99 (d, J = 8.1 Hz, 1H), 5.91 (d, J = 8.1 Hz, 1H), 4.30 (d, J = 10.4 Hz, 2H), 4.29 (s, 1H), 4.23 (s, 2H), 3.61 (t, J = 7.9 Hz, 1H), 2.82 (q, J = 7.2 Hz, 2H), 2.69 (s, 3H), 2.65-2.55 (m, 2H), 2.40-2.30 (m, 5H), 1.15 (t, J = 7.2 Hz, 3H). |
| 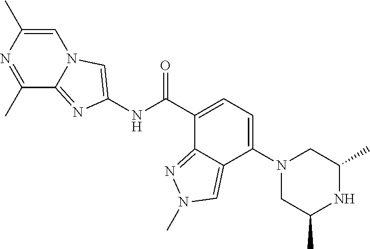 [SMILES: Cc1cn2cc(NC(=O)c3ccc(N4C[C@H](C)N[C@@H](C)C4)c4cn(C)nc34)nc2c(C)n1] Compound 549 | The compound was obtained as a solid (Compound 549, 17 mg, 33% yield) following the general procedure. LCMS (ES, m/z): 433 [M + H]⁺. ¹H NMR (300 MHz, DMSO-d₆) δ 11.44 (s, 1H), 8.70 (s, 1H), 8.32 (d, J = 2.3 Hz, 2H), 8.02 (d, J = 8.1 Hz, 1H), 6.47 (d, J = 8.2 Hz, 1H), 4.32 (s, 3H), 3.42 (dd, J = 11.8, 3.1 Hz, 2H), 3.27 (td, J = 6.3, 2.9 Hz, 2H), 3.11 (dd, J = 11.8, 6.4 Hz, 2H), 2.70 (s, 3H), 2.37 (s, 3H), 2.17 (s, 1H), 1.16 (d, J = 6.3 Hz, 6H). |
| 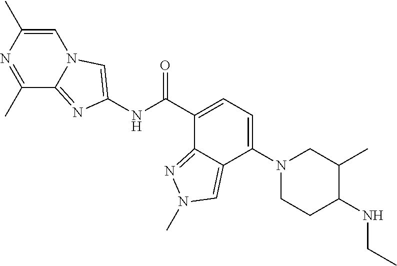 [SMILES: CCNC1CCN(c2ccc(C(=O)Nc3cn4cc(C)nc(C)c4n3)c3nn(C)cc23)CC1C] Compound 604 | The compound was obtained as a solid (Compound 604, 2.4 mg, 5% yield) following the general procedure. LCMS (ES, m/z): 461 [M + H]⁺. ¹H NMR (300 MHz, Methanol-d₄) δ 8.49 (s, 1H), 8.33 (s, 1H), 8.19 (s, 1H), 8.10 (d, J = 8.2 Hz, 1H), 6.53 (d, J = 8.3 Hz, 1H), 4.38 (s, 3H), 3.93 (d, J = 12.6 Hz, 2H), 3.20-3.01 (m, 2H), 2.91- 2.82 (m, 1H), 2.80 (s, 3H), 2.78-2.67 (m, 2H), 2.46 (s, 3H), 2.27 (s, 1H), 1.88-1.80 (m, 2H), 1.26-1.09 (m, 6H). |
| 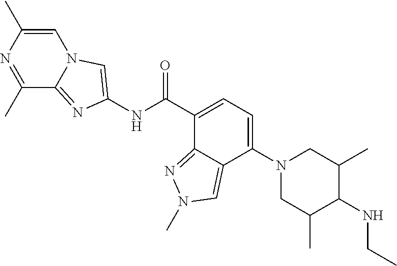 [SMILES: CCNC1C(C)CN(c2ccc(C(=O)Nc3cn4cc(C)nc(C)c4n3)c3nn(C)cc23)CC1C] Compound 686 | The compound was obtained as a solid (Compound 686, 15 mg, 28% yield) following the general procedure. LCMS (ES, m/z): 475 [M + H]⁺. ¹H NMR (400 MHz, DMSO-d₆) δ 11.51 (s, 1H), 8.77 (s, 1H), 8.40-8.30 (m, 4H), 8.06 (d, J = 8.1 Hz, 1H), 6.57 (d, J = 8.2 Hz, 1H), 4.34 (s, 3H), 3.87 (dd, J = 23.8, 12.6 Hz, 2H), 3.16-3.10 (m, 3H), 3.08-3.00 (m, 1H), 2.82-2.71 (m, 4H), 2.40 (s, 4H), 2.25 (d, J = 9.1 Hz, 1H), 1.29 (t, J = 7.1 Hz, 3H), 1.12 (dd, J = 6.7, 3.6 Hz, 6H). |
| 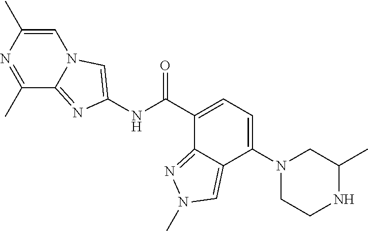 [SMILES: Cc1cn2cc(NC(=O)c3ccc(N4CCNC(C)C4)c4cn(C)nc34)nc2c(C)n1] Compound 557 | The compound was obtained as a solid (Compound 557, 18 mg, 38% yield) following the general procedure. LCMS (ES, m/z): 419 [M + H]⁺. ¹H NMR (300 MHz, DMSO-d₆) δ 11.46 (s, 1H), 8.82 (s, 1H), 8.32 (d, J = 2.3 Hz, 2H), 8.03 (d, J = 8.1 Hz, 1H), 6.50 (d, J = 8.2 Hz, 1H), 4.31 (s, 3H), 3.78 (d, J = 9.7 Hz, 2H), 3.05-2.93 (m, 4H), 2.90 (d, J = 8.5 Hz, 3H), 2.57 (d, J = 11.0 Hz, 1H), 2.38 (s, 3H), 1.07 (d, J = 6.2 Hz, 3H). |

### Example 71: Synthesis of Compounds 537, 565, 592-594

### General Procedure B

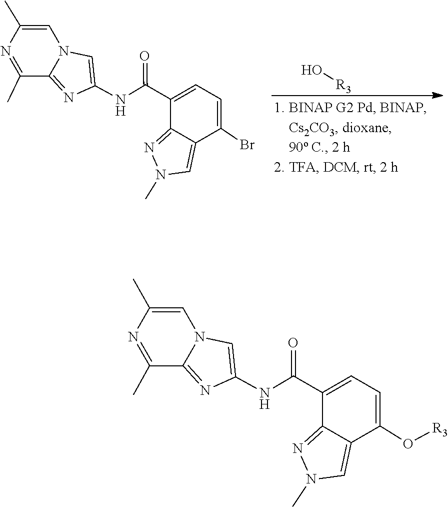

To a stirred mixture of 4-bromo-N-(6,8-dimethylimidazo[1,2-a]pyrazin-2-yl)-2-methyl-indazole-7-carboxamide (50 mg, 125.6 μmol, 1 eq) and alcohol (150.8 μmol, 1.2 eq) in Dioxane (1 mL) were added Cs₂CO₃ (81.6 mg, 251.2 μmol, 2 eq), BINAP (15.6 mg, 25.1 μmol, 0.2 eq) and BINAP Pd G2 (11.3 mg, 12.6 μmol, 0.1 eq), and the reaction was stirred for 2 h at 90° C. under N₂. The resulting mixture was concentrated under reduced pressure and purified by silica gel column chromatography (9% MeOH in DCM) to afford the product. The product was dissolved in DCM (1.5 mL). To this solution was added TFA (0.5 mL) at room temperature, and the reaction was stirred for 2 h. The resulting mixture was concentrated under reduced pressure and adjusted to pH 8 with saturated NaHCO₃ (aq.). The residue was purified by reversed-phase flash chromatography to give the final product.

An analogous method was followed to obtain the following compounds.

| Compounds | Characterization |
|---|---|
| 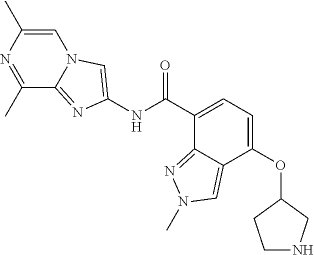 [SMILES: Cc1cn2cc(NC(=O)c3ccc(OC4CCNC4)c4cn(C)nc34)nc2c(C)n1] Compound 537 | The compound was obtained as a solid (Compound 537, 19 mg, 33% yield) following the general procedure. LCMS (ES, m/z): 406 [M + H]⁺. ¹H NMR (300 MHz, DMSO-d₆) δ 11.34 (s, 1H), 8.69 (d, J = 16.5 Hz, 1H), 8.34 (d, J = 5.0 Hz, 2H), 8.13 (d, J = 8.1 Hz, 1H), 6.71 (dd, J = 20.8, 8.0 Hz, 1H), 5.13 (s, 1H), 4.32 (s, 3H), 3.60-3.51 (m, 1H), 3.19 (dd, J = 12.7, 5.3 Hz, 2H), 2.95 (t, J = 11.8 Hz, 2H), 2.85-2.80 (m, 1H), 2.70 (s, 3H), 2.38 (s, 3H), 2.13 (dd, J = 13.8, 6.9 Hz, 1H), 1.90-1.80 (m, 1H). |
| 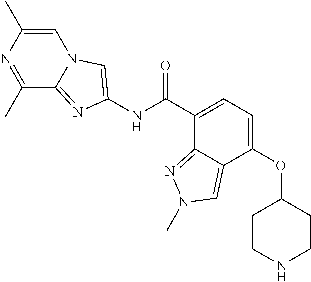 [SMILES: Cc1cn2cc(NC(=O)c3ccc(OC4CCNCC4)c4cn(C)nc34)nc2c(C)n1] Compound 565 | The compound was obtained as a solid (Compound 565, 9.2 mg, 15% yield) following the general procedure. LCMS (ES, m/z): 420 [M + H]⁺. ¹H NMR (400 MHz, DMSO-d₆) δ 11.33 (s, 1H), 8.69 (s, 1H), 8.33 (d, J = 4.4 Hz, 2H), 8.12 (d, J = 8.1 Hz, 1H), 6.75 (d, J = 8.2 Hz, 1H), 4.79-4.72 (m, 1H), 4.32 (s, 3H), 3.05-2.95 (m, 2H), 2.75-2.60 (m, 5H), 2.38 (s, 3H), 2.07- 1.99 (m, 2H), 1.59 (d, J = 10.6 Hz, 2H). |
| 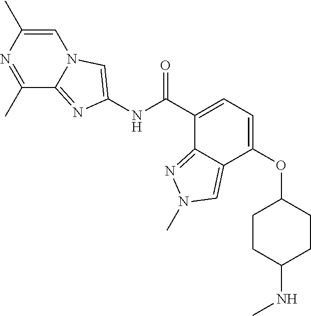 [SMILES: CNC1CCC(Oc2ccc(C(=O)Nc3cn4cc(C)nc(C)c4n3)c3nn(C)cc23)CC1] Compound 592 | The compound was obtained as a solid (Compound 592, 9.8 mg, 20% yield) following the general procedure. LCMS (ES, m/z):448 [M + H]⁺. ¹H NMR (400 MHz, DMSO-d₆) δ 11.44 (s, 1H), 8.74 (s, 2H), 8.65 (s, 1H), 8.50 (d, J = 16.5 Hz, 2H), 8.12 (d, J = 8.1 Hz, 1H), 6.82 (d, J = 8.2 Hz, 1H), 4.75 (s, 1H), 4.30 (s, 3H), 3.05 (s, 1H), 2.78 (s, 3H), 2.56 (t, J = 5.5 Hz, 3H), 2.42 (s, 3H), 2.27 (s, 2H), 2.12 (s, 2H), 1.53 (p, J = 12.5, 11.3 Hz, 4H). |
| 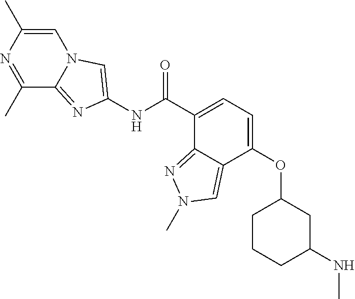 [SMILES: CNC1CCCC(Oc2ccc(C(=O)Nc3cn4cc(C)nc(C)c4n3)c3nn(C)cc23)C1] Compound 593 | The compound was obtained as a solid (Compound 593, 16 mg, 24% yield) following the general procedure. LCMS (ES, m/z): 448 [M + H]⁺. ¹H NMR (400 MHz, DMSO-d₆) δ 11.37 (s, 1H), 8.68 (s, 1H), 8.58 (s, 2H), 8.39 (d, J = 4.4 Hz, 2H), 8.14 (d, J = 8.1 Hz, 1H), 6.83 (d, J = 8.3 Hz, 1H), 4.72 (s, 1H), 4.33 (s, 3H), 3.23 (s, 1H), 2.73 (s, 3H), 2.61 (t, J = 5.4 Hz, 3H), 2.56-2.55 (m, 1H), 2.40 (s, 3H), 2.21 (d, J = 11.2 Hz, 1H), 2.05 (d, J = 11.8 Hz, 1H), 1.91 (d, J = 13.0 Hz, 1H), 1.50-1.20 (m, 4H). |
| 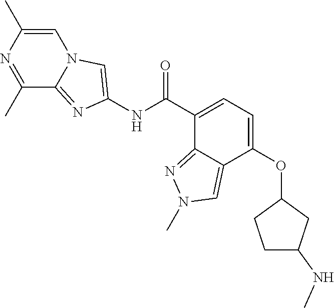 [SMILES: CNC1CCC(Oc2ccc(C(=O)Nc3cn4cc(C)nc(C)c4n3)c3nn(C)cc23)C1] Compound 594 | The compound was obtained as a solid (Compound 594, 6 mg, 10% yield) following the general procedure. LCMS (ES, m/z): 434 [M + H]⁺. ¹H NMR (400 MHz, DMSO-d₆) δ 11.51 (s, 1H), 9.25-9.15 (m, 2H), 8.69 (s, 1H), 8.62 (s, 1H), 8.56 (s, 2H), 8.16 (d, J = 8.0 Hz, 1H), 6.72 (d, J = 8.2 Hz, 1H), 5.26 (d, J = 6.6 Hz, 1H), 4.32 (s, 3H), 3.68 (q, J = 6.9 Hz, 1H), 2.86 (s, 3H), 2.56 (t, J = 5.6 Hz, 3H), 2.48 (s, 3H), 2.35 (ddt, J = 21.6, 14.9, 8.1 Hz, 2H), 2.27-2.15 (m, 2H), 1.95-1.79 (m, 2H). |

### Example 72: Synthesis of Compounds 506-515, 518-519, 538-548, 553, 568, 577-578, 602, and 605

### General Procedure C

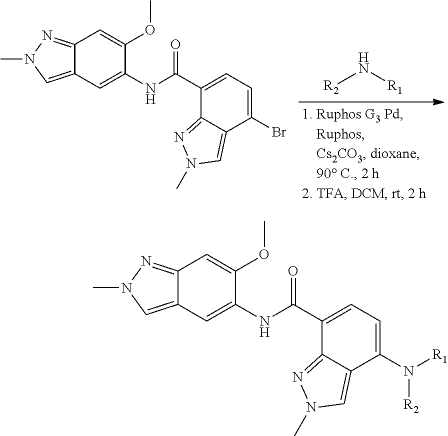

To a mixture of 4-bromo-N-(6-methoxy-2-methyl-2H-indazol-5-yl)-2-methyl-2H-indazole-7-carboxamide (50 mg, 121 μmol, 1 eq) and amine (145.3 μmol, 1.2 eq) in dioxane (1 mL) were added Cs₂CO₃ (78.6 mg, 242 μmol, 2 eq), RuPhos (11.2 mg, 24.2 μmol, 0.2 eq) and RuPhos Pd G3 (10.1 mg, 12.1 μmol, 0.1 eq), and the reaction was stirred for 2 h at 90° C. under N₂. The resulting mixture was concentrated under reduced pressure and purified by silica gel column chromatography (9% MeOH in DCM) to afford the product. The product was dissolved in DCM (2 mL). To this solution was added TFA (1 mL) at room temperature, and the reaction was stirred for 2 h. The resulting mixture was concentrated under reduced pressure and adjusted to pH 8 with saturated NaHCO₃ (aq.). The residue was purified by reversed-phase flash chromatography to give the final product.

An analogous method was followed to obtain the following compounds.

| Compounds | Characterization |
|---|---|
| 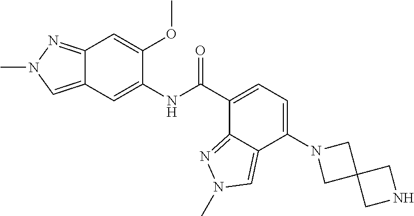 [SMILES: COc1cc2nn(C)cc2cc1NC(=O)c1ccc(N2CC3(CNC3)C2)c2cn(C)nc12] Compound 506 | The compound was obtained as a solid (Compound 506, 13 mg, 23% yield) following the general procedure. LCMS (ES, m/z): 432 [M + H]⁺. ¹H NMR (400 MHz, DMSO-d₆) δ 11.47 (s, 1H), 8.82 (s, 1H), 8.62 (s, 1H), 8.17 (s, 1H), 7.95 (d, J = 8.0 Hz, 1H), 7.05 (s, 1H), 5.89 (d, J = 8.1 Hz, 1H), 4.30-4.29 (m, 4H), 4.29 (s, 3H), 4.07- 4.05 (m, 4H), 4.03 (s, 3H), 3.69 (s, 3H). |
| 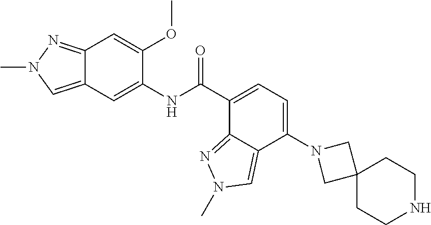 [SMILES: COc1cc2nn(C)cc2cc1NC(=O)c1ccc(N2CC3(CCNCC3)C2)c2cn(C)nc12] Compound 507 | The compound was obtained as a solid (Compound 507, 15 mg, 26% yield) following the general procedures. LCMS (ES, m/z): 460 [M + H]⁺. ¹H NMR (400 MHz, DMSO-d₆) δ 11.46 (s, 1H), 8.83 (s, 1H), 8.66 (s, 1H), 8.16 (s, 1H), 7.94 (d, J = 8.0 Hz, 1H), 7.05 (s, 1H), 5.87 (d, J = 8.1 Hz, 1H), 4.26 (s, 3H), 4.07 (s, 3H), 4.03 (s, 3H), 3.95 (s, 4H), 2.72-2.65 (m, 4H), 1.75-1.68 (m, 4H). |
| 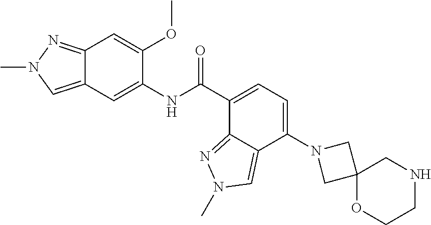 [SMILES: COc1cc2nn(C)cc2cc1NC(=O)c1ccc(N2CC3(CNCCO3)C2)c2cn(C)nc12] Compound 508 | The compound was obtained as a solid (Compound 508, 13 mg, 22% yield) following the general procedures. LCMS (ES, m/z): 462 [M + H]⁺. ¹H NMR (400 MHz, DMSO-d₆) δ 11.47 (s, 1H), 8.83 (s, 1H), 8.67 (s, 1H), 8.17 (s, 1H), 7.96 (d, J = 8.0 Hz, 1H), 7.06 (s, 1H), 5.93 (d, J = 8.1 Hz, 1H), 4.27 (s, 3H), 4.17 (d, J = 8.6 Hz, 2H), 4.08 (s, 3H), 4.07-3.99 (m, 5H), 3.65-3.58 (m, 2H), 2.92 (s, 2H), 2.70 (t, J = 4.7 Hz, 2H), 2.69-2.64 (m, 1H). |
| 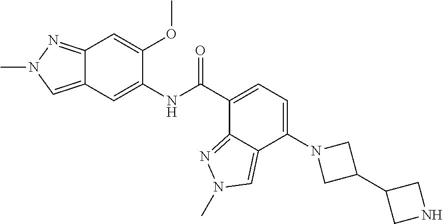 [SMILES: COc1cc2nn(C)cc2cc1NC(=O)c1ccc(N2CC(C3CNC3)C2)c2cn(C)nc12] Compound 509 | The compound was obtained as a solid (Compound 509, 11 mg, 19% yield) following the general procedure. LCMS (ES, m/z): 446 [M + H]⁺. ¹H NMR (400 MHz, DMSO-d₆) δ 11.46 (s, 1H), 8.82 (s, 1H), 8.64 (s, 1H), 8.16 (s, 1H), 7.95 (d, J = 8.0 Hz, 1H), 7.05 (s, 1H), 5.87 (d, J = 8.1 Hz, 1H), 4.33 (t, J = 8.0 Hz, 2H), 4.26 (s, 3H), 4.07 (s, 3H), 4.03 (s, 3H), 3.95 (t, J = 6.8 Hz, 2H), 3.61 (t, J = 7.8 Hz, 2H), 3.30-3.21 (m, 2H), 3.10 (s, 1H), 2.97- 2.95 (m, 1H). |
| 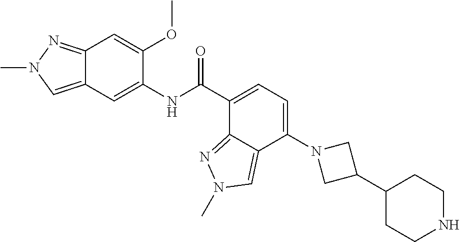 [SMILES: COc1cc2nn(C)cc2cc1NC(=O)c1ccc(N2CC(C3CCNCC3)C2)c2cn(C)nc12] Compound 510 | The compound was obtained as a solid (Compound 510, 11 mg, 19% yield) following the general procedure. LCMS (ES, m/z): 474 [M + H]⁺. ¹H NMR (400 MHz, DMSO-d₆) δ 11.46 (s, 1H), 8.82 (s, 1H), 8.65 (s, 1H), 8.16 (s, 1H), 7.94 (d, J = 8.0 Hz, 1H), 7.05 (s, 1H), 5.86 (d, J = 8.1 Hz, 1H), 4.31-4.27 (m, 2H), 4.26 (s, 3H), 4.07 (s, 3H), 4.03 (s, 3H), 3.95 (d, J = 7.3 Hz, 2H), 2.97 (d, J = 11.8 Hz, 2H), 2.59-2.54 (m, 1H), 2.50-2.34 (m, 3H), 1.07-0.95 (m, 2H). |
| 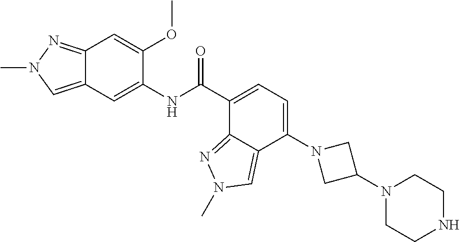 [SMILES: COc1cc2nn(C)cc2cc1NC(=O)c1ccc(N2CC(N3CCNCC3)C2)c2cn(C)nc12] Compound 511 | The compound was obtained as a solid (Compound 511, 14 mg, 23% yield) following the general procedure. LCMS (ES, m/z): 474 [M + H]⁺. ¹H NMR (400 MHz, DMSO-d₆) δ 11.46 (s, 1H), 8.82 (s, 1H), 8.65 (s, 1H), 8.17 (s, 1H), 7.95 (d, J = 8.0 Hz, 1H), 7.05 (s, 1H), 5.89 (d, J = 8.1 Hz, 1H), 4.31-4.25 (m, 5H), 4.10-4.00 (m, 8H), 2.73 (t, J = 4.7 Hz, 4H), 2.32-2.25 (m, 4H). |
| 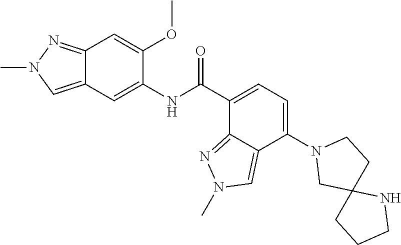 [SMILES: COc1cc2nn(C)cc2cc1NC(=O)c1ccc(N2CCC3(CCCN3)C2)c2cn(C)nc12] Compound 512 | The compound was obtained as a solid (Compound 512, 12 mg, 20% yield) following the general procedure. LCMS (ES, m/z): 460 [M + H]⁺. ¹H NMR (400 MHz, DMSO-d₆) δ 11.52 (s, 1H), 8.82 (s, 1H), 8.78 (s, 1H), 8.16 (s, 1H), 7.94 (d, J = 8.2 Hz, 1H), 7.05 (s, 1H), 5.97 (d, J = 8.3 Hz, 1H), 4.26 (s, 3H), 4.07 (s, 3H), 4.03 (s, 3H), 3.72 (dd, J = 16.1, 8.4 Hz, 2H), 3.59-3.54 (m, 2H), 2.90 (hept, J = 4.6, 4.2 Hz, 2H), 2.00 (t, J = 6.9 Hz, 2H), 1.83-1.69 (m, 4H). |
| 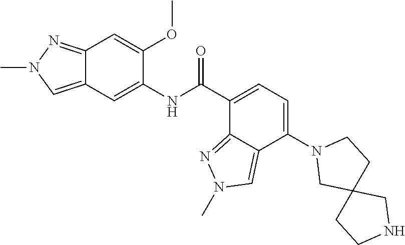 [SMILES: COc1cc2nn(C)cc2cc1NC(=O)c1ccc(N2CCC3(CCNC3)C2)c2cn(C)nc12] Compound 513 | The compound was obtained as a solid (Compound 513, 14 mg, 25% yield) following the general procedure. LCMS (ES, m/z): 460 [M + H]⁺. ¹H NMR (400 MHz, DMSO-d₆) δ 11.53 (d, J = 3.1 Hz, 1H), 8.81 (d, J = 16.8 Hz, 2H), 8.16 (s, 1H), 7.95 (dd, J = 8.2, 2.0 Hz, 1H), 7.05 (s, 1H), 6.01 (dd, J = 10.7, 8.3 Hz, 1H), 4.25 (s, 3H), 4.07 (s, 3H), 4.03 (s, 3H), 3.76-3.65 (m, 2H), 3.64-3.50 (m, 2H), 3.45-3.42 (m, 1H), 3.41-3.40 (m, 1H), 2.91 (t, J = 7.1 Hz, 1H), 2.80-2.69 (m, 1H), 2.08-1.89 (m, 2H), 1.84- 1.67 (m, 2H). |
| 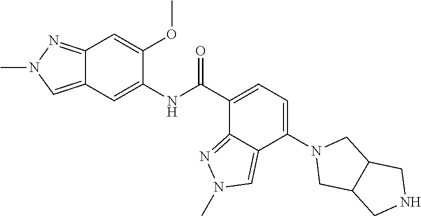 [SMILES: COc1cc2nn(C)cc2cc1NC(=O)c1ccc(N2CC3CNCC3C2)c2cn(C)nc12] Compound 514 | The compound was obtained as a solid (Compound 514, 12 mg, 21% yield) following the general procedure. LCMS (ES, m/z): 446 [M + H]⁺. ¹H NMR (400 MHz, DMSO-d₆) δ 11.55 (s, 1H), 8.83 (d, J = 5.6 Hz, 2H), 8.16 (s, 1H), 7.95 (d, J = 8.2 Hz, 1H), 7.05 (s, 1H), 6.03 (d, J = 8.3 Hz, 1H), 4.26 (s, 3H), 4.07 (s, 3H), 4.03 (s, 3H), 3.88 (t, J = 8.7 Hz, 2H), 3.48 (dd, J = 10.9, 2.8 Hz, 2H), 2.97 (td, J = 11.7, 9.6, 5.8 Hz, 4H), 2.78-2.70 (m, 2H). |
| 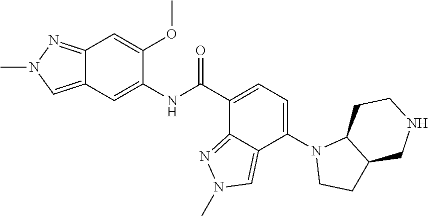 [SMILES: COc1cc2nn(C)cc2cc1NC(=O)c1ccc(N2CC[C@@H]3CNCC[C@@H]32)c2cn(C)nc12] Compound 515 | The compound was obtained as a solid (Compound 515, 12 mg, 20% yield) following the general procedure. LCMS (ES, m/z): 460 [M + H]⁺. ¹H NMR (400 MHz, DMSO-d₆) δ 11.54 (s, 1H), 8.83 (s, 1H), 8.75 (s, 1H), 8.15 (s, 1H), 7.92 (d, J = 8.3 Hz, 1H), 7.05 (s, 1H), 6.07 (d, J = 8.4 Hz, 1H), 4.26 (s, 3H), 4.17 (s, 1H), 4.07 (s, 3H), 4.03 (s, 3H), 3.80 (t, J = 9.5 Hz, 1H), 3.70 (q, J = 9.1 Hz, 1H), 3.02-2.89 (m, 2H), 2.86 (d, J = 12.4 Hz, 1H), 2.59-2.51 (m, 1H), 2.39 (t, J = 10.8 Hz, 1H), 2.28-2.26 (m, 1H), 2.06-1.97 (m, 1H), 1.92-1.89 (m, 1H), 1.34-1.30 (m, 1H). |
| 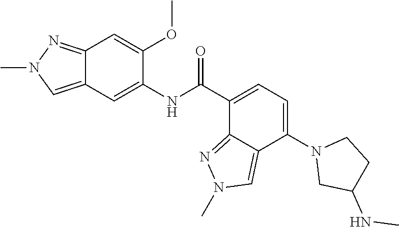 Compound 518 | The compound was obtained as a solid (Compound 518, 13 mg, 22% yield) following the general procedure. LCMS (ES, m/z): 434 [M + H]⁺. ¹H NMR (400 MHz, DMSO-d₆) δ 11.53 (s, 1H), 8.82 (s, 1H), 8.78 (s, 1H), 8.16 (s, 1H), 7.94 (d, J = 8.2 Hz, 1H), 7.05 (s, 1H), 5.99 (d, J = 8.3 Hz, 1H), 4.26 (s, 3H), 4.07 (s, 3H), 4.03 (s, 3H), 3.75 (ddd, J = 23.5, 12.2, 6.7 Hz, 2H), 3.64 (q, J = 7.7 Hz, 1H), 3.42 (dd, J = 10.3, 4.1 Hz, 1H), 2.35 (s, 3H), 2.15 (dq, J = 13.0, 6.9 Hz, 1H), 2.01-1.97 (m, 1H), 1.96- 1.87 (m, 1H). |
| 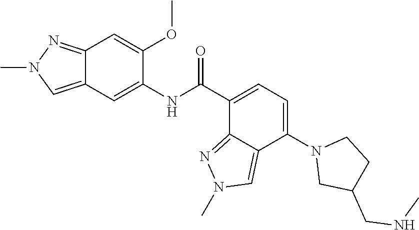 Compound 538 | The compound was obtained as a solid (Compound 538, 13 mg, 27% yield) following the general procedure. LCMS (ES, m/z): 448 [M + H]⁺. ¹H NMR (400 MHz, DMSO-d₆) δ 11.53 (s, 1H), 8.82 (s, 1H), 8.78 (s, 1H), 8.16 (s, 1H), 7.94 (d, J = 8.3 Hz, 1H), 7.05 (s, 1H), 6.00 (d, J = 8.3 Hz, 1H), 4.26 (s, 3H), 4.07 (s, 3H), 4.03 (s, 3H), 3.78 (t, J = 8.4 Hz, 1H), 3.71-3.64 (m, 2H), 3.40 (t, J = 8.4 Hz, 1H), 2.65-2.51 (m, 3H), 2.33 (s, 3H), 2.17- 2.11 (m, 1H), 1.86-1.73 (m, 1H). |
| 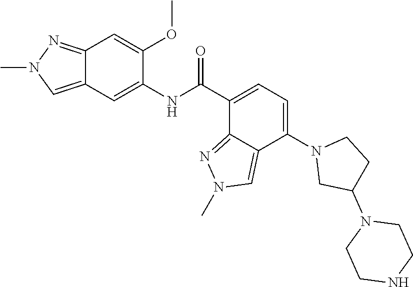 [SMILES: COc1cc2nn(C)cc2cc1NC(=O)c1ccc(N2CCC(N3CCNCC3)C2)c2cn(C)nc12] Compound 539 | The compound was obtained as a solid (Compound 539, 11 mg, 23% yield) following the general procedure. LCMS (ES, m/z): 489 [M + H]⁺. ¹H NMR (400 MHz, DMSO-d₆) δ 11.54 (s, 1H), 8.82 (s, 2H), 8.16 (s, 1H), 7.94 (d, J = 8.2 Hz, 1H), 7.05 (s, 1H), 6.02 (d, J = 8.4 Hz, 1H), 4.27 (s, 3H), 4.07 (s, 3H), 4.03 (s, 3H), 3.88 (t, J = 8.4 Hz, 1H), 3.73 (t, J = 9.1 Hz, 1H), 3.61 (q, J = 9.2 Hz, 1H), 3.47 (t, J = 9.0 Hz, 1H), 3.02-2.91 (m, 1H), 2.75 (t, J = 4.9 Hz, 4H), 2.55-2.45 (m, 4H), 2.30-2.23 (m, 1H), 1.95-1.83 (m, 1H). |
| 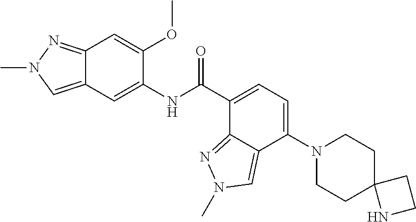 [SMILES: COc1cc2nn(C)cc2cc1NC(=O)c1ccc(N2CCC3(CCN3)CC2)c2cn(C)nc12] Compound 540 | The compound was obtained as a solid (Compound 540, 10 mg, 20% yield) following the general procedure. LCMS (ES, m/z): 460 [M + H]⁺. ¹H NMR (400 MHz, DMSO-d₆) δ 11.61 (s, 1H), 8.84 (s, 1H), 8.75 (d, J = 17.5 Hz, 1H), 8.18 (s, 1H), 7.99 (d, J = 8.1 Hz, 1H), 7.07 (s, 1H), 6.49 (d, J = 8.1 Hz, 1H), 4.29 (s, 3H), 4.07 (s, 3H), 4.03 (s, 3H), 3.95-3.75 (m, 1H), 3.52-3.42 (m, 2H), 3.38- 3.36 (m, 2H), 3.33-3.22 (m, 1H), 2.07 (t, J = 7.5 Hz, 2H), 1.94-1.85 (m, 2H), 1.85-1.76 (m, 2H). |
| 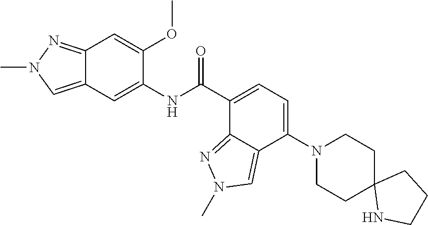 [SMILES: COc1cc2nn(C)cc2cc1NC(=O)c1ccc(N2CCC3(CCCN3)CC2)c2cn(C)nc12] Compound 541 | The compound was obtained as a solid (Compound 541, 16 mg, 33% yield) following the general procedure. LCMS (ES, m/z): 474 [M + H]⁺. ¹H NMR (400 MHz, DMSO-d₆) δ 11.64 (s, 1H), 9.24 (s, 2H), 8.84 (s, 1H), 8.80 (s, 1H), 8.23 (s, 1H), 8.02 (d, J = 8.0 Hz, 1H), 7.09 (s, 1H), 6.54 (d, J = 8.1 Hz, 1H), 4.31 (s, 3H), 4.09 (s, 3H), 4.05 (s, 3H), 3.75 (d, J = 13.3 Hz, 2H), 3.35-3.23 (m, 4H), 2.14-1.94 (m, 8H). |
| 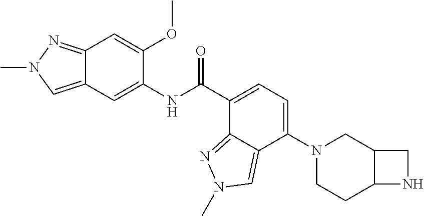 [SMILES: COc1cc2nn(C)cc2cc1NC(=O)c1ccc(N2CCC3NCC3C2)c2cn(C)nc12] Compound 542 | The compound was obtained as a solid (Compound 542, 14 mg, 28% yield) following the general procedure. LCMS (ES, m/z): 446 [M + H]⁺. ¹H NMR (400 MHz, DMSO-d₆) δ 11.59 (d, J = 2.8 Hz, 1H), 8.89-8.78 (m, 2H), 8.17 (s, 1H), 7.98 (d, J = 8.3 Hz, 1H), 7.06 (s, 1H), 6.28 (dd, J = 12.8, 8.4 Hz, 1H), 4.27 (d, J = 2.0 Hz, 3H), 4.07-3.94 (m, 8H), 3.85-3.53 (m, 4H), 3.18 (dd, J = 7.8, 5.1 Hz, 1H), 3.02-3.01 (m, 1H), 2.25-1.75 (m, 2H). |
| 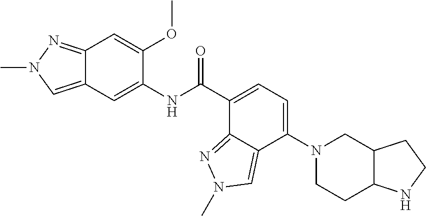 [SMILES: COc1cc2nn(C)cc2cc1NC(=O)c1ccc(N2CCC3NCCC3C2)c2cn(C)nc12] Compound 543 | The compound was obtained as a solid (Compound 543, 14 mg, 28% yield) following the general procedure. LCMS (ES, m/z): 460 [M + H]⁺. ¹H NMR (400 MHz, DMSO-d₆) δ 11.59 (d, J = 3.2 Hz, 1H), 8.84 (s, 1H), 8.73 (d, J = 13.2 Hz, 1H), 8.18 (d, J = 4.4 Hz, 1H), 7.97 (dd, J = 8.2, 5.2 Hz, 1H), 7.06 (d, J = 3.4 Hz, 1H), 6.37 (dd, J = 21.9, 8.3 Hz, 1H), 4.28 (s, 3H), 4.07 (s, 3H), 4.03 (s, 3H), 3.95-3.71 (m, 1H), 3.64 (dd, J = 12.7, 5.3 Hz, 1H), 3.55- 3.45 (m, 2H), 3.31-3.17 (m, 2H), 2.97 (ddd, J = 10.1, 8.0, 4.3 Hz, 1H), 2.75 (dt, J = 10.1, 7.8 Hz, 1H), 2.36 (d, J = 7.6 Hz, 1H), 2.05-1.93 (m, 1H), 1.87 (ddd, J = 12.5, 8.2, 4.2 Hz, 1H), 1.80-1.69 (m, 1H), 1.45 (dt, J = 12.4, 6.4 Hz, 1H). |
| 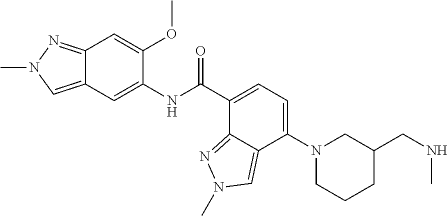 Compound 519 | The compound was obtained as a solid (Compound 519, 14 mg, 24% yield) following the general procedure. LCMS (ES, m/z): 462 [M + H]⁺. ¹H NMR (400 MHz, DMSO-d₆) δ 11.61 (s, 1H), 8.84 (s, 1H), 8.71 (s, 1H), 8.18 (s, 1H), 7.99 (d, J = 8.0 Hz, 1H), 7.07 (s, 1H), 6.48 (d, J = 8.2 Hz, 1H), 4.29 (s, 3H), 4.07 (s, 3H), 4.03 (s, 3H), 3.93 (d, J = 12.6 Hz, 2H), 2.94 (dd, J = 13.1, 10.7 Hz, 2H), 2.46 (d, J = 6.6 Hz, 2H), 2.33 (s, 3H), 1.87 (d, J = 12.5 Hz, 2H), 1.72-1.65 (m, 1H), 1.36 (tt, J = 12.3, 6.1 Hz, 2H). |
| 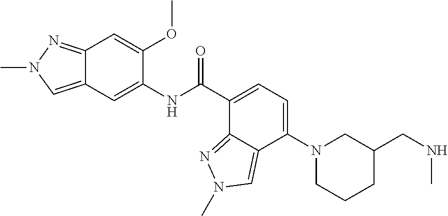 Compound 545 | The compound was obtained as a solid (Compound 545, 14 mg, 28% yield) following the general procedure. LCMS (ES, m/z): 462 [M + H]⁺. ¹H NMR (400 MHz, DMSO-d₆) δ 11.61 (s, 1H), 8.84 (s, 1H), 8.77 (s, 1H), 8.18 (s, 1H), 7.99 (d, J = 8.1 Hz, 1H), 7.07 (s, 1H), 6.47 (d, J = 8.2 Hz, 1H), 4.29 (s, 3H), 4.07 (s, 3H), 4.03 (s, 3H), 3.86-3.72 (m, 2H), 3.03 (ddd, J = 12.8, 10.3, 3.0 Hz, 1H), 2.94-2.84 (m, 1H), 2.55-2.54 (m, 1H), 2.51-2.40 (m, 1H), 2.33 (s, 3H), 1.90-1.73 (m, 3H), 1.73-1.60 (m, 1H), 1.33-1.20 (m, 1H). |
| 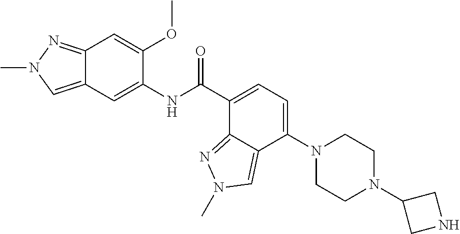 [SMILES: COc1cc2nn(C)cc2cc1NC(=O)c1ccc(N2CCN(C3CNC3)CC2)c2cn(C)nc12] Compound 544 | The compound was obtained as a solid (Compound 544, 13 mg, 25% yield) following the general procedure. LCMS (ES, m/z): 475 [M + H]⁺. ¹H NMR (400 MHz, DMSO-d₆) δ 11.63 (s, 1H), 8.84 (s, 1H), 8.77 (s, 1H), 8.19 (s, 1H), 8.01 (d, J = 7.9 Hz, 1H), 7.07 (s, 1H), 6.50 (d, J = 8.1 Hz, 1H), 4.29 (s, 3H), 4.07 (s, 3H), 4.03 (s, 3H), 3.90-3.75 (m, 1H), 3.60-3.55 (m, 2H), 3.40 (s, 6H), 3.18-3.14 (m, 1H), 2.60-2.47 (m, 3H). |
| 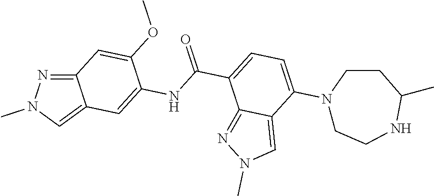 Compound 546 | The compound was obtained as a solid (Compound 546, 15 mg, 30% yield) following the general procedure. LCMS (ES, m/z): 448 [M + H]⁺. ¹H NMR (400 MHz, DMSO-d₆) δ 11.59 (s, 1H), 8.83 (s, 1H), 8.68 (s, 1H), 8.16 (s, 1H), 7.94 (d, J = 8.3 Hz, 1H), 7.05 (s, 1H), 6.21 (d, J = 8.5 Hz, 1H), 4.27 (s, 3H), 4.07 (s, 3H), 4.03 (s, 3H), 3.88-3.79 (m, 2H), 3.75 (dd, J = 9.4, 3.3 Hz, 1H), 3.64-3.50 (m, 1H), 3.21-3.15 (m, 1H), 2.95 (ddd, J = 14.0, 10.7, 3.2 Hz, 1H), 2.70 (td, J = 6.7, 3.2 Hz, 1H), 2.03- 1.93 (m, 1H), 1.52 (qd, J = 10.2, 9.8, 4.8 Hz, 1H), 1.04 (d, J = 6.3 Hz, 3H). |
| 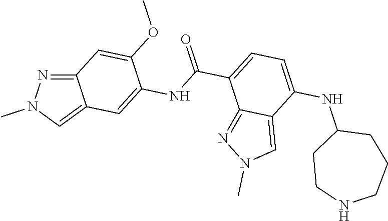 [SMILES: COc1cc2nn(C)cc2cc1NC(=O)c1ccc(NC2CCCNCC2)c2cn(C)nc12] Compound 547 | The compound was obtained as a solid (Compound 547, 13 mg, 26% yield) following the general procedure. LCMS (ES, m/z): 448 [M + H]⁺. ¹H NMR (400 MHz, DMSO-d₆) δ 11.39 (s, 1H), 8.82 (s, 1H), 8.64 (d, J = 11.4 Hz, 1H), 8.16 (s, 1H), 7.94 (d, J = 8.1 Hz, 1H), 7.05 (s, 1H), 6.80 (t, J = 9.3 Hz, 1H), 6.06 (dd, J = 11.5, 8.2 Hz, 1H), 4.26 (s, 3H), 4.07 (s, 3H), 4.03 (s, 3H), 3.77 (d, J = 4.5 Hz, 1H), 3.65-3.62 (m, 1H), 3.55-3.50 (m, 1H), 2.95- 2.72 (m, 3H), 2.10-1.93 (m, 2H), 1.85-1.51 (m, 5H). |
| 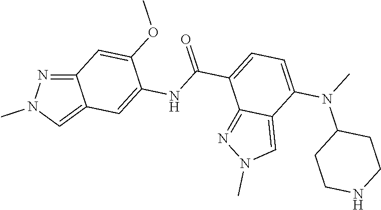 [SMILES: COc1cc2nn(C)cc2cc1NC(=O)c1ccc(N(C)C2CCNCC2)c2cn(C)nc12] Compound 548 | The compound was obtained as a solid (Compound 548, 7 mg, 14% yield) following the general procedure. LCMS (ES, m/z): 448 [M + H]⁺. ¹H NMR (400 MHz, DMSO-d₆) δ 11.60 (s, 1H), 8.84 (s, 1H), 8.63 (s, 1H), 8.17 (s, 1H), 7.96 (d, J = 8.2 Hz, 1H), 7.06 (s, 1H), 6.32 (d, J = 8.4 Hz, 1H), 4.30 (s, 3H), 4.07 (s, 3H), 4.03 (s, 3H), 3.99 (s, 1H), 3.05-3.01 (m, 5H), 2.66 (t, J = 10.6 Hz, 2H), 1.81-1.68 (m, 4H). |
| 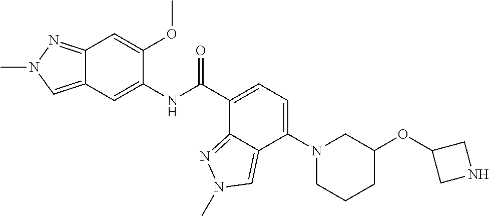 [SMILES: COc1cc2nn(C)cc2cc1NC(=O)c1ccc(N2CCCC(OC3CNC3)C2)c2cn(C)nc12] Compound 602 | The compound was obtained as a solid (Compound 602, 19 mg, 42% yield) following the general procedure. LCMS (ES, m/z): 490 [M + H]⁺. ¹H NMR (400 MHz, DMSO-d₆) δ 11.62 (s, 1H), 8.84 (s, 1H), 8.69 (s, 1H), 8.18 (s, 1H), 8.00 (d, J = 8.1 Hz, 1H), 7.07 (s, 1H), 6.49 (d, J = 8.1 Hz, 1H), 4.44 (q, J = 6.2 Hz, 1H), 4.31 (s, 3H), 4.07-4.01 (m, 7H), 3.79-3.71 (m, 1H), 3.66-3.62 (m, 2H), 3.47-3.35 (m, 3H), 3.09 (ddd, J = 12.9, 10.1, 2.9 Hz, 1H), 2.97 (dd, J = 12.1, 8.4 Hz, 1H), 2.04-1.95 (m, 1H), 1.90- 1.82 (m, 1H), 1.69-1.61 (m, 1H), 1.47 (q, J = 10.6 Hz, 1H). |
| 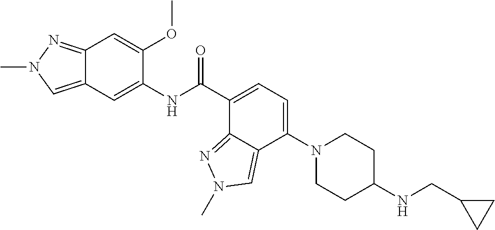 [SMILES: COc1cc2nn(C)cc2cc1NC(=O)c1ccc(N2CCC(NCC3CC3)CC2)c2cn(C)nc12] Compound 577 | The compound was obtained as a solid (Compound 577, 10 mg, 21% yield) following the general procedure. LCMS (ES, m/z): 488 [M + H]⁺. ¹H NMR (400 MHz, DMSO-d₆) δ 11.61 (s, 1H), 8.84 (s, 1H), 8.73 (s, 1H), 8.18 (s, 1H), 7.99 (d, J = 8.1 Hz, 1H), 7.07 (s, 1H), 6.47 (d, J = 8.1 Hz, 1H), 4.29 (s, 3H), 4.07 (s, 3H), 4.03 (s, 3H), 3.86 (d, J = 12.8 Hz, 2H), 3.02 (t, J = 11.5 Hz, 2H), 2.68 (s, 1H), 2.46 (d, J = 6.6 Hz, 2H), 1.97 (d, J = 12.2 Hz, 2H), 1.75-1.55 (m, 1H), 1.48-1.43 (m, 2H), 0.89 (dd, J = 14.0, 7.0 Hz, 1H), 0.46-0.35 (m, 2H), 0.16-0.05 (m, 2H). |
| 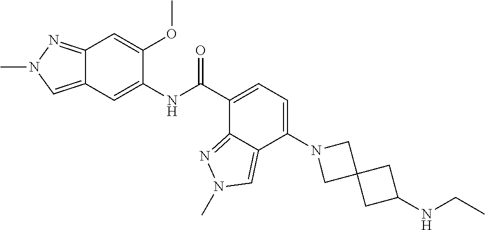 [SMILES: CCNC1CC2(C1)CN(c1ccc(C(=O)Nc3cc4cn(C)nc4cc3OC)c3nn(C)cc13)C2] Compound 578 | The compound was obtained as a solid (Compound 578, 8 mg, 16% yield) following the general procedure. LCMS (ES, m/z): 474 [M + H]⁺. ¹H NMR (400 MHz, DMSO-d₆) δ 11.46 (s, 1H), 8.82 (s, 1H), 8.60 (s, 1H), 8.16 (s, 1H), 7.94 (d, J = 8.1 Hz, 1H), 7.05 (s, 1H), 5.85 (d, J = 8.1 Hz, 1H), 4.25-4.21 (m, 5H), 4.14 (s, 2H), 4.07 (s, 3H), 4.03 (s, 3H), 3.15-3.05 (m, 1H), 2.49-2.43 (m, 4H), 1.95 (t, J = 10.0 Hz, 2H), 1.75-1.65 (m, 1H), 1.00 (t, J = 7.1 Hz, 3H). |
| 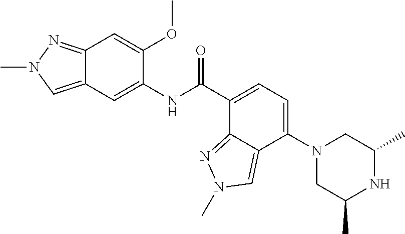 Compound 553 | The compound was obtained as a solid (Compound 553, 9 mg, 18% yield) following the general procedure. LCMS (ES, m/z): 448 [M + H]⁺. ¹H NMR (400 MHz, DMSO-d₆) δ 11.62 (s, 1H), 8.84 (s, 1H), 8.64 (s, 1H), 8.18 (s, 1H), 7.99 (d, J = 8.1 Hz, 1H), 7.07 (s, 1H), 6.45 (d, J = 8.1 Hz, 1H), 4.31 (s, 3H), 4.07 (s, 3H), 4.03 (s, 3H), 3.38-3.33 (m, 2H), 3.29-3.23 (m, 2H), 3.08 (dd, J = 11.8, 6.3 Hz, 2H), 1.17 (d, J = 6.4 Hz, 6H). |
| 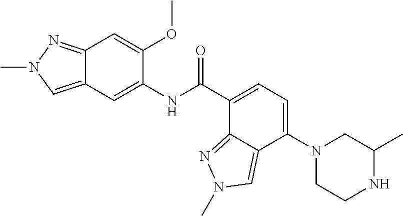 Compound 568 | The compound was obtained as a solid (Compound 568, 12 mg, 24% yield) following the general procedure. LCMS (ES, m/z): 434 [M + H]⁺. ¹H NMR (400 MHz, DMSO-d₆) δ 11.62 (s, 1H), 8.84 (s, 1H), 8.76 (s, 1H), 8.18 (s, 1H), 8.00 (d, J = 8.0 Hz, 1H), 7.07 (s, 1H), 6.47 (d, J = 8.1 Hz, 1H), 4.29 (s, 3H), 4.07 (s, 3H), 4.03 (s, 3H), 3.75 (d, J = 11.6 Hz, 2H), 3.02-2.95 (m, 1H), 2.97- 2.82 (m, 3H), 2.52-2.50 (m, 1H), 2.42-2.33 (m, 1H), 1.07 (d, J = 6.3 Hz, 3H). |
| 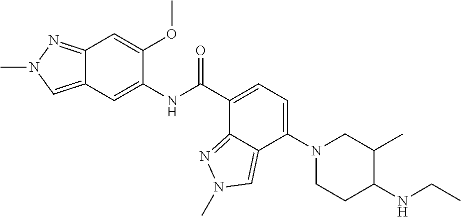 [SMILES: CCNC1CCN(c2ccc(C(=O)Nc3cc4cn(C)nc4cc3OC)c3nn(C)cc23)CC1C] Compound 605 | The compound was obtained as a solid (Compound 605, 8 mg, 16% yield) following the general procedure. LCMS (ES, m/z): 476 [M + H]⁺. ¹H NMR (400 MHz, DMSO-d₆) δ 11.61 (s, 1H), 8.84 (s, 1H), 8.72-8.67 (m, 1H), 8.18 (s, 1H), 7.99 (dd, J = 8.1, 1.5 Hz, 1H), 7.07 (s, 1H), 6.47 (dd, J = 8.1, 2.3 Hz, 1H), 4.30 (d, J = 2.9 Hz, 3H), 4.07 (s, 3H), 4.03 (s, 3H), 3.95-3.82 (m, 1H), 3.68 (dt, J = 11.7, 5.9 Hz, 1H), 3.13-2.96 (m, 2H), 2.74 (dd, J = 14.8, 8.0 Hz, 1H), 2.58 (q, J = 7.4, 7.0 Hz, 2H), 2.10- 2.02 (m, 1H), 1.75-1.66 (m, 2H), 1.45-1.42 (m, 1H), 1.10- 1.00 (m, 6H). |
| 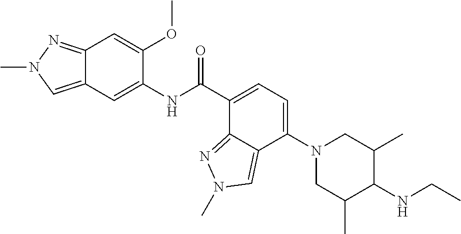 [SMILES: CCNC1C(C)CN(c2ccc(C(=O)Nc3cc4cn(C)nc4cc3OC)c3nn(C)cc23)CC1C] Compound 687 | The compound was obtained as a solid (Compound 687, 11 mg, 21% yield) following the general procedure. LCMS (ES, m/z): 490 [M + H]⁺. ¹H NMR (400 MHz, DMSO-d₆) δ 11.62 (s, 1H), 8.84 (s, 1H), 8.65 (s, 1H), 8.18 (s, 1H), 7.98 (d, J = 8.0 Hz, 1H), 7.07 (s, 1H), 6.46 (d, J = 8.1 Hz, 1H), 4.31 (s, 3H), 4.07 (s, 3H), 4.03 (s, 3H), 3.81-3.65 (m, 2H), 3.09 (dd, J = 12.6, 2.9 Hz, 1H), 2.78-2.67 (m, 2H), 2.50-2.42 (m, 1H), 2.39-2.31 (m, 1H), 2.17-2.14 (m, 1H), 1.86-1.80 (m, 1H), 1.10-0.97 (m, 9H). |

### Example 73: Synthesis of Compounds 566-567, 595-596, and 603

### General Procedure D

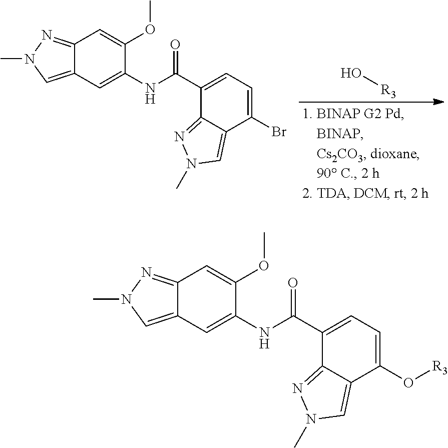

To a stirred mixture of 4-bromo-N-(6-methoxy-2-methyl-2H-indazol-5-yl)-2-methyl-2H-indazole-7-carboxamide (50 mg, 121 μmol, 1 eq) and alcohol (145.3 μmol, 1.2 eq) in Dioxane (1 mL) were added Cs₂CO₃ (78.6 mg, 242 μmol, 2 eq), BINAP (15 mg, 24.2 μmol) and BINAP Pd G2 (11.3 mg, 12.1 μmol), and the reaction was stirred for 2 h at 90° C. under N₂. The resulting mixture was concentrated under reduced pressure and purified by silica gel column chromatography (9% MeOH in DCM) to afford the product. The product was dissolved in DCM (2 mL). To this solution was added TFA (1 mL) at room temperature, and the reaction was stirred for 2 h. The resulting mixture was concentrated under reduced pressure and adjusted to pH 8 with saturated NaHCO₃ (aq.). The residue was purified by reversed-phase flash chromatography to give the final product.

An analogous method was followed to obtain the following compounds.

| Compounds | Characterization |
|---|---|
| 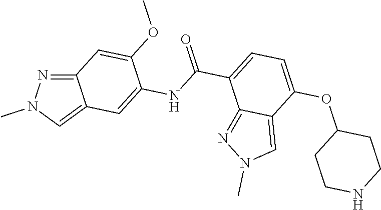 [SMILES: COc1cc2nn(C)cc2cc1NC(=O)c1ccc(OC2CCNCC2)c2cn(C)nc12] | The compound was obtained as a solid (Compound 566, 12 mg, 25% yield) following the general procedure. LCMS (ES, m/z): 435 [M + H]⁺. ¹H NMR (400 MHZ, DMSO-d₆) δ 11.51 (s, 1H), 8.84 (s, 1H), 8.65 (s, 1H), 8.20 (s, 1H), 8.07 (d, J = 8.0 Hz, 1H), 7.08 (s, 1H), 6.71 (d, J = 8.2 Hz, 1H), 4.73 (dt, J = 9.2, 4.8 Hz, 1H), 4.30 (s, 3H), 4.07 (s, 3H), 4.03 (s, 3H), 3.04-2.96 (m, 2H), 2.67-2.59 (m, 2H), 2.03 (d, J = 12.2 Hz, 2H), 1.65-1.52 (m, 1H). |
| Compound 566 | |
| 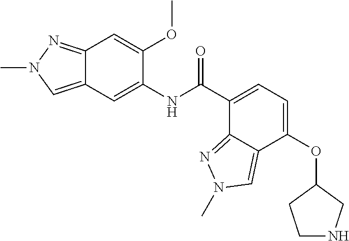 [SMILES: COc1cc2nn(C)cc2cc1NC(=O)c1ccc(OC2CCNC2)c2cn(C)nc12] | The compound was obtained as a solid (Compound 567, 12 mg, 25% yield) following the general procedure. LCMS (ES, m/z): 421 [M + H]⁺. ¹H NMR (400 MHZ, DMSO-d₆) δ 11.51 (s, 1H), 8.84 (s, 1H), 8.61 (s, 1H), 8.20 (s, 1H), 8.08 (d, J = 8.0 Hz, 1H), 7.08 (s, 1H), 6.63 (d, J = 8.1 Hz, 1H), 5.11-5.07 (m, 1H), 4.30 (s, 3H), 4.07 (s, 3H), 4.03 (s, 3H), 3.18 (dd, J = 12.5, 5.4 Hz, 1H), 3.00-2.89 (m, 2H), 2.83 (ddd, J = 10.9, 8.0, 4.9 Hz, 1H), 2.12 (dq, J = 14.2, 7.3 Hz, 1H), 1.87 (dd, J = 12.9, 6.2 Hz, 1H). |
| Compound 567 | |
| 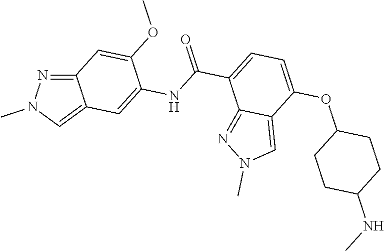 [SMILES: CNC1CCC(Oc2ccc(C(=O)Nc3cc4cn(C)nc4cc3OC)c3nn(C)cc23)CC1] | The compound was obtained as a solid (Compound 595, 13 mg, 30% yield) following the general procedure. LCMS (ES, m/z): 463 [M + H]⁺. ¹H NMR (400 MHZ, DMSO-d₆) δ 11.51 (s, 1H), 8.89 (s, 2H), 8.84 (s, 1H), 8.61 (s, 1H), 8.22 (s, 1H), 8.08 (d, J = 8.0 Hz, 1H), 7.09 (s, 1H), 6.78 (d, J = 8.2 Hz, 1H), 4.62 (s, 1H), 4.30 (s, 3H), 4.09 (s, 3H), 4.05 (s, 3H), 3.07 (s, 1H), 2.57 (t, J = 5.4 Hz, 3H), 2.29 (d, J = 10.6 Hz, 2H), 2.16 (d, J = 10.9 Hz, 2H), 1.57 (q, J = 12.4, 11.8 Hz, 4H). |
| Compound 595 | |
| 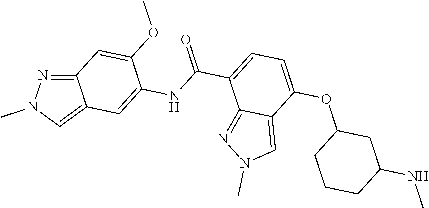 | The compound was obtained as a solid (Compound 596, 12 mg, 29% yield) following the general procedure. LCMS (ES, m/z): 463 [M + H]⁺. ¹H NMR (400 MHZ, DMSO-d₆) δ 11.49 (s, 1H), 8.81 (s, 1H), 8.69 (s, 2H), 8.61 (s, 1H), 8.18 (s, 1H), 8.06 (d, J = 8.0 Hz, 1H), 7.06 (s, 1H), 6.76 (d, J = 8.2 Hz, 1H), 4.75-4.65 (m, 1H), 4.29 (s, 3H), 4.08 (s, 3H), 4.05 (s, 3H), 3.21-3.15 (m, 1H), 2.60-2.55 (m, 4H), 2.19 (d, J = 11.2 Hz, 1H), 2.04 (d, J = 11.8 Hz, 1H), 1.89 (d, J = 12.5 Hz, 1H), 1.41 (ddq, J = 43.0, 22.7, 11.6 Hz, 4H). |
| Compound 596 | |
| 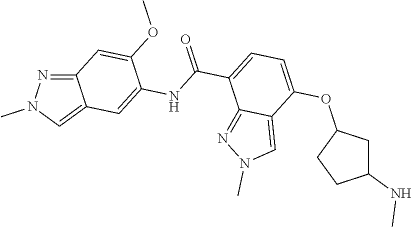 [SMILES: CNC1CCC(Oc2ccc(C(=O)Nc3cc4cn(C)nc4cc3OC)c3nn(C)cc23)C1] | The compound was obtained as a solid (Compound 603, 4 mg, 8% yield) following the general procedure. LCMS (ES, m/z): 449 [M + H]⁺. ¹H NMR (400 MHZ, DMSO-d₆) δ 11.52 (s, 1H), 9.17 (s, 2H), 8.85 (s, 1H), 8.62 (s, 1H), 8.24 (s, 1H), 8.10 (d, J = 7.9 Hz, 1H), 7.09 (s, 1H), 6.66 (d, J = 8.2 Hz, 1H), 5.23 (s, 1H), 4.30 (s, 3H), 4.08 (s, 3H), 4.05 (s, 3H), 3.70-3.65 (m, 1H), 2.60-2.53 (m, 3H), 2.34 (ddd, J = 20.9, 13.6, 7.1 Hz, 2H), 2.24-2.15 (m, 2H), 1.88 (ddt, J = 20.5, 13.5, 7.2 Hz, 2H). |
| Compound 603 | |

### Example 74: Synthesis of C4

### Synthesis of C1

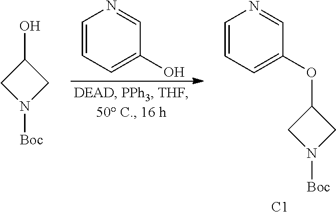

To a stirred mixture of tert-butyl 3-hydroxyazetidine-1-carboxylate (5 g, 28.9 mmol) and pyridin-3-ol (4.12 g, 43.3 mmol) in THF (50 mL) were added PPh₃ (9.1 g, 34.6 mmol), DEAD (6.03 g, 34.6 mmol) at room temperature under N₂. The resulting mixture was stirred for 16 h at 50° C. under N₂. The resulting mixture was allowed to cool to room temperature, diluted with water (50 mL), and extracted with EtOAc (3×50 mL). The combined organic layers were washed with water (100 mL), brine (80 mL), dried over anhydrous Na₂SO₄, and filtered. The filtrate was concentrated under reduced pressure and purified by silica gel column chromatography (75% EtOAc in PE) to afford tert-butyl 3-(3-pyridyloxy) azetidine-1-carboxylate (C1, 1.5 g, 5.99 mmol, 21% yield) as an oil. LCMS (ES, m/z): 251 [M+H]⁺.

### Synthesis of C2

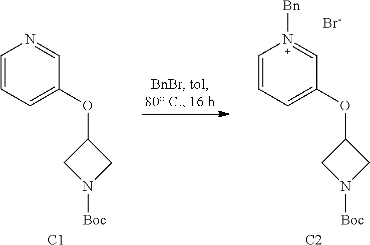

To a stirred mixture of tert-butyl 3-(3-pyridyloxy) azetidine-1-carboxylate (1.5 g, 5.99 mmol) in Toluene (30 mL) were added BnBr (1.23 g, 7.2 mmol) at room temperature under N₂. The resulting mixture was stirred for 16 h at 80° C. under N₂. The mixture was allowed to cool down to room temperature and concentrated under reduced pressure to remove toluene. The crude product was triturated with petroleum ether (100 mL), then filtered to give the filter cake as a crude product 1-benzyl-3-((1-(tert-butoxycarbonyl) azetidin-3-yl)oxy)pyridin-1-ium bromide (1.8 g, 4.3 mmol, 71% yield) was obtained as a solid. LCMS (ES, m/z): 341 [M+H]⁺.

### Synthesis of C3

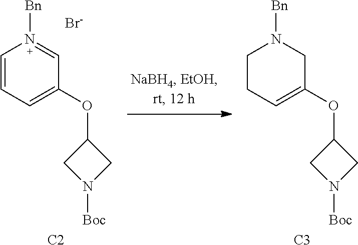

To a stirred mixture of 1-benzyl-3-((1-(tert-butoxycarbonyl) azetidin-3-yl)oxy)pyridin-1-ium bromide (1.8 g, 4.3 mmol) in EtOH (20 mL) were added NaBH₄ (126.2 mg, 4.3 mmol), and the reaction was stirred for 12 h at room temperature. The resulting mixture was quenched with ice-water (60 mL) and extracted with EtOAc (3×40 mL). The combined organic layers were washed with water (40 mL), brine (40 mL), dried over anhydrous Na₂SO₄, and filtered. The filtrate was concentrated under reduced pressure and purified by silica gel column chromatography (25% EtOAc in PE) to afford tert-butyl 3-((1-benzyl-1,2,5,6-tetrahydropyridin-3-yl)oxy) azetidine-1-carboxylate (0.57 g, 1.65 mmol 70% yield) as a solid. LCMS (ES, m/z): 345 [M+H]⁺.

### Synthesis of C4

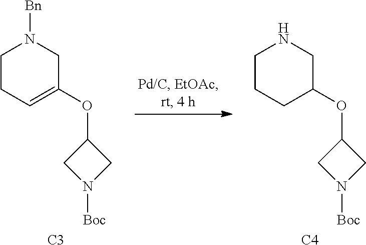 [SMILES: CC(C)(C)OC(=O)N1CC(OC2=CCCN(Cc3ccccc3)C2)C1 ; CC(C)(C)OC(=O)N1CC(OC2CCCNC2)C1 ; "C3" ; "C4"]

To a solution of tert-butyl 3-[(1-benzyl-3,6-dihydro-2H-pyridin-5-yl)oxy]azetidine-1-carboxylate (0.57 g, 1.65 mmol) in EtOAc (10 mL) was added Pd/C (0.1 g). The reaction was stirred for 4 h at room temperature under hydrogen atmosphere. The resulting mixture was filtered, the filter cake was washed with EtOAc (3×5 ml). The solution was concentrated under reduced pressure to afford tert-butyl 3-(3-piperidyloxy) azetidine-1-carboxylate (0.33 g, 1.3 mmol, 78% yield) as an oil. LCMS (ES, m/z): 257 [M+H]⁺, 298 [M+H+CH₃CN]⁺.

### Example 75: Synthesis of Amine C10

### Synthesis of C5

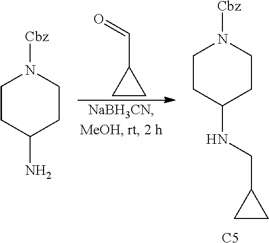 [SMILES: NC1CCN(C(=O)OCc2ccccc2)CC1 ; O=CC1CC1 ; O=C(OCc1ccccc1)N1CCC(NCC2CC2)CC1 ; "C5"]

To a stirred mixture of benzyl 4-aminopiperidine-1-carboxylate (3 g, 12.8 mmol) and cyclopropanecarbaldehyde (1.08 g, 15.37 mmol) in MeOH (30 mL) were added NaBH₃CN (1.9 g, 30.24 mmol), and the reaction was stirred for 2 h at room temperature. The resulting mixture was diluted with water (20 mL) and extracted with EtOAc (3×30 mL). The combined organic layers were washed with brine (50 mL), dried over anhydrous Na₂SO₄, and filtered. The filtrate was concentrated under reduced pressure and purified by silica gel column chromatography (25% EtOAc in PE) to afford benzyl 4-(cyclopropylmethylamino)-piperidine-1-carboxylate (1.9 g, 6.6 mmol, 52% yield) as a solid. LCMS (ES, m/z): 289 [M+H]⁺.

### Synthesis of C6

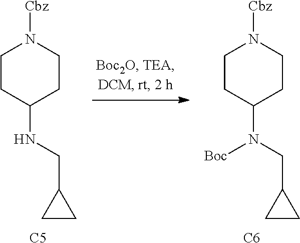 [SMILES: O=C(OCc1ccccc1)N1CCC(NCC2CC2)CC1 ; CC(C)(C)OC(=O)N(CC1CC1)C1CCN(C(=O)OCc2ccccc2)CC1 ; "C5" ; "C6"]

To a solution of benzyl 4-(cyclopropylmethylamino)piperidine-1-carboxylate (1.9 g, 6.6 mmol) in DCM (20 mL) was added Boc₂O (1.7 g, 7.9 mmol) and TEA (2 g, 19.8 mmol), and the reaction was stirred for 2 h at rt. The resulting mixture was diluted with water (20 mL) and extracted with EtOAc (3×20 mL). The combined organic layers were washed with water (40 mL), brine (40 mL), dried over anhydrous Na₂SO₄, and filtered. The filtrate was concentrated under reduced pressure to afford benzyl 4-[tert-butoxycarbonyl(cyclopropylmethyl)amino]-piperidine-1-carboxylate (1.8 g, 4.6 mmol, 70% yield) as a solid. LCMS (ES, m/z): 389 [M+H]⁺.

### Synthesis of C7

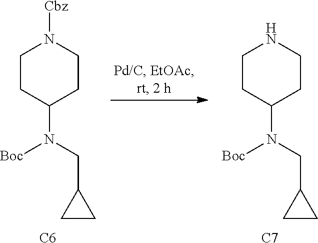 [SMILES: CC(C)(C)OC(=O)N(CC1CC1)C1CCN(C(=O)OCc2ccccc2)CC1 ; CC(C)(C)OC(=O)N(CC1CC1)C1CCNCC1 ; "C6" ; "C7"]

To a solution of benzyl 4-[tert-butoxycarbonyl(cyclopropylmethyl)amino]piperidine-1-carboxylate (0.3 g, 772.2 μmol) in EtOAc (5 mL) was added Pd/C (82.1 mg). The reaction was stirred for 2 h at rt under hydrogen atmosphere. The resulting mixture was filtered, the filter cake was washed with EtOAc (3×5 mL). The filtrate was concentrated under reduced pressure to afford tert-butyl N-(cyclopropylmethyl)-N-(4-piperidyl)carbamate (C7, 0.15 g, 590 μmol, 76% yield) as an oil. LCMS (ES, m/z): 255 [M+H]⁺.

### Synthesis of C8

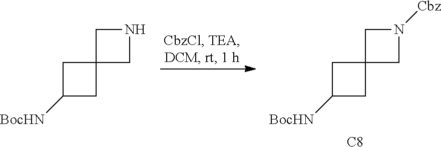 [SMILES: CC(C)(C)OC(=O)NC1CC2(CNC2)C1 ; "CbzCl, TEA,DCM, rt, 1 h" ; CC(C)(C)OC(=O)NC1CC2(C1)CN(C(=O)OCc1ccccc1)C2 ; "C8"]

To a stirred mixture of tert-butyl N-(2-azaspiro[3.3]heptan-6-yl)carbamate (2 g, 9.4 mmol) and TEA (2.9 g, 28.3 mmol) in DCM (20 mL) were added CbzCl (1.6 g, 9.4 mmol), and the reaction was stirred for 1 h at room temperature. The resulting mixture was diluted with water (20 mL) and extracted with EtOAc (3×20 mL). The combined organic layers were washed with water (40 mL), brine (40 mL), dried over anhydrous Na₂SO₄, and filtered. The filtrate was concentrated under reduced pressure and purified by silica gel column chromatography (25% EtOAc in PE) to afford benzyl 6-(tert-butoxycarbonylamino)-2-azaspiro[3.3]heptane-2-carboxylate (1.7 g, 4.9 mmol, 52% yield) as a solid. LCMS (ES, m/z): 347 [M+H]⁺.

### Synthesis of C9

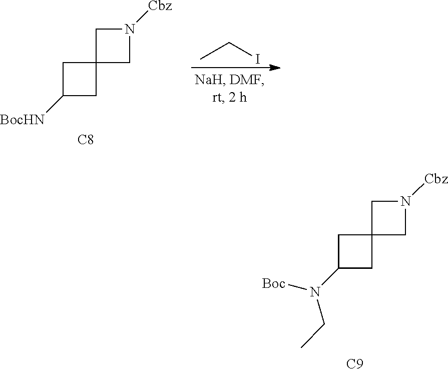

To a stirred mixture of benzyl 6-(tert-butoxycarbonylamino)-2-azaspiro[3.3]heptane-2-carboxylate (1.7 g, 4.9 mmol) and NaH (60% dispersion in oil) (235.5 mg, 9.8 mmol) in DMF (20 mL) were added iodoethane (918.4 mg, 5.9 mmol), and the reaction was stirred for 2 h at room temperature. The resulting mixture was diluted with water (10 mL) and extracted with EtOAc (3×20 mL). The combined organic layers were washed with water (40 mL), brine (40 mL), dried over anhydrous Na₂SO₄, and filtered. The filtrate was concentrated under reduced pressure and purified by silica gel column chromatography (33% EtOAc in PE) to afford benzyl 6-[tert-butoxycarbonyl(ethyl)amino]-2-azaspiro[3.3]heptane-2-carboxylate (1.7 g, 4.5 mmol, 93% yield) as a solid. LCMS (ES, m/z): 375 [M+H]⁺.

### Synthesis of C10

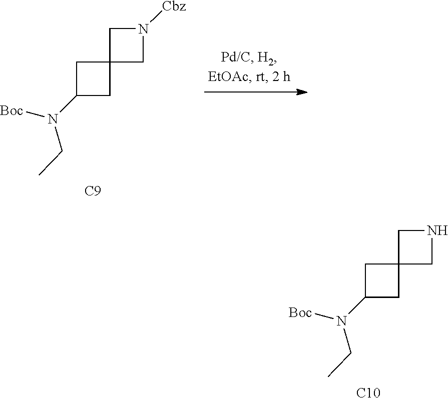 [SMILES: CCN(C(=O)OC(C)(C)C)C1CC2(C1)CN(C(=O)OCc1ccccc1)C2 ; "C9" ; CCN(C(=O)OC(C)(C)C)C1CC2(CNC2)C1 ; "C10"]

To a solution of benzyl 6-[tert-butoxycarbonyl(ethyl)amino]-2-azaspiro[3.3]heptane-2-carboxylate (0.5 g, 1.34 mmol) in EtOAc (5 mL) was added Pd/C (0.05 g), and the reaction was stirred for 2 h at rt under hydrogen atmosphere. The resulting mixture was filtered, the filter cake was washed with EtOAc (3×5 mL). The filtrate was concentrated under reduced pressure to afford tert-butyl N-(cyclopropylmethyl)-N-(4-piperidyl)carbamate (0.15 g, 589.7 μmol, 76% yield) as an oil. LCMS (ES, m/z): 241 [M+H]⁺.

### Example 76: Synthesis of C13

### Synthesis of C11

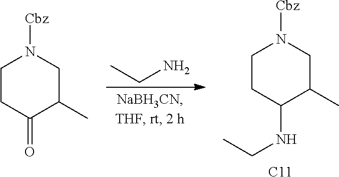

To a stirred mixture of benzyl 3-methyl-4-oxo-piperidine-1-carboxylate (2 g, 8.1 mmol) and ethanamine (4.9 mL, 9.7 mmol) in THF (20 mL) were added NaBH₃CN (1.5 g, 24.3 mmol), and the reaction was stirred for 2 h at room temperature. The resulting mixture was diluted with water (20 mL) and extracted with EtOAc (3×20 mL). The combined organic layers were washed with water (40 mL), brine (40 mL), dried over anhydrous Na₂SO₄, and filtered. The filtrate was concentrated under reduced pressure and purified by silica gel column chromatography (20% EtOAc in PE) to afford benzyl 4-(ethylamino)-3-methyl-piperidine-1-carboxylate (1.54 g, 5.6 mmol, 69% yield) as an oil. LCMS (ES, m/z): 277 [M+H]⁺.

### Synthesis of C12

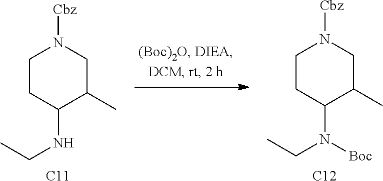 [SMILES: CCNC1CCN(C(=O)OCc2ccccc2)CC1C ; CCN(C(=O)OC(C)(C)C)C1CCN(C(=O)OCc2ccccc2)CC1C ; "C11" ; "C12"]

To a solution of benzyl 4-(ethylamino)-3-methyl-piperidine-1-carboxylate (2.2 g, 7.96 mmol) in DCM (20 mL) was added DIEA (3.1 g, 23.9 mmol) and Boc₂O (1.74 g, 7.96 mmol). The reaction was stirred for 2 h at rt. The resulting mixture was diluted with water (20 mL) and extracted with DCM (3×20 mL). The combined organic layers were washed with water (40 mL), brine (40 mL), dried over anhydrous Na₂SO₄, and filtered. The filtrate was concentrated under reduced pressure, to afford benzyl 4-[tert-butoxycarbonyl(ethyl)amino]-3-methyl-piperidine-1-carboxylate (1.3 g, 3.5 mmol, 43% yield) as a solid. LCMS (ES, m/z): 377 [M+H]⁺.

### Synthesis of C13

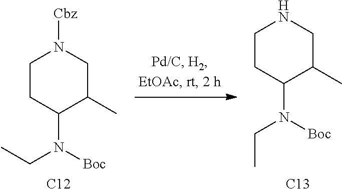

To a solution of benzyl 4-[tert-butoxycarbonyl(ethyl)amino]-3-methyl-piperidine-1-carboxylate (0.5 g, 1.3 mmol) in EtOAc (5 mL) was added Pd/C (0.1 g), and the reaction was stirred for 2 h at rt under hydrogen atmosphere. The resulting mixture was filtered, the filter cake was washed with EtOAc (3×5 mL). The filtrate was concentrated under reduced pressure to afford tert-butyl N-ethyl-N-(3-methyl-4-piperidyl)carbamate (0.3 g, 1.24 mmol, 93% yield) as an oil. LCMS (ES, m/z): 243 [M+H]⁺.

### Example 77: Synthesis of C16

### Synthesis of C14

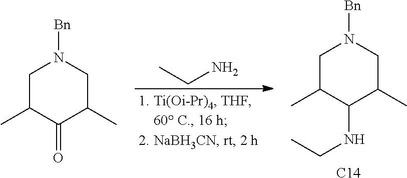

To a stirred mixture of 1-benzyl-3,5-dimethyl-piperidin-4-one (0.65 g, 2.99 mmol) and ethanamine (162 mg, 3.6 mmol) in THF (6 mL) were added Ti(Oi-Pr)₄ (1.7 g, 5.98 mmol), and the reaction was stirred for 16 h at 60° C. To the above mixture was added NaBH₃CN (563.9 mg, 8.97 mmol) and stirred for 2 h at room temperature. The resulting mixture was diluted with water (10 mL) and extracted with EtOAc (3×20 mL). The combined organic layers were washed with water (40 mL), brine (40 mL), dried over anhydrous Na₂SO₄, and filtered. The filtrate was concentrated under reduced pressure and purified by silica gel column chromatography (25% EtOAc in PE) to afford 1-benzyl-N-ethyl-3,5-dimethyl-piperidin-4-amine (0.2 g, 811.7 μmol, 27% yield) as an oil. LCMS (ES, m/z): 247 [M+H]⁺.

### Synthesis of C15

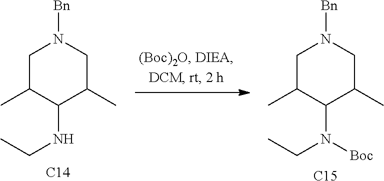

To a solution of 1-benzyl-N-ethyl-3,5-dimethyl-piperidin-4-amine (0.2 g, 811.7 μmol) in DCM (4 mL) was added DIEA (314.7 mg, 2.4 mmol) and Boc₂O (177.2 mg, 811.7 μmol). The reaction was stirred for 2 h at rt. The resulting mixture was diluted with water (4 mL) and extracted with DCM (3×4 mL). The combined organic layers were washed with water (10 mL), brine (10 mL), dried over anhydrous Na₂SO₄, and filtered. The filtrate was concentrated under reduced pressure, to afford tert-butyl N-(1-benzyl-3,5-dimethyl-4-piperidyl)-N-ethyl-carbamate (0.2 g, 577.2 μmol, 71% yield) as a solid. LCMS (ES, m/z): 347 [M+H]⁺.

### Synthesis of C16

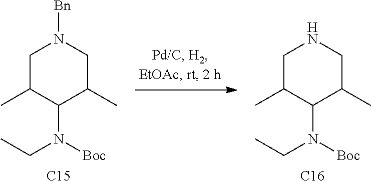

To a solution of tert-butyl N-(1-benzyl-3,5-dimethyl-4-piperidyl)-N-ethyl-carbamate (0.2 g, 577.2 μmol) in EtOAc (10 mL) was added Pd/C (0.03 g). The reaction was stirred for 2 h at rt under hydrogen atmosphere. The resulting mixture was filtered, the filter cake was washed with EtOAc (3×5 mL). The filtrate was concentrated under reduced pressure to afford tert-butyl N-(3,5-dimethyl-4-piperidyl)-N-ethyl-carbamate (0.13 g, 507 μmol, 88% yield) as an oil. LCMS (ES, m/z): 257 [M+H]⁺.

### Example 78: Synthesis of C19-C₂₁

### Synthesis of C17

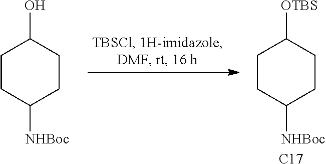

To a stirred solution of tert-butyl N-(4-hydroxycyclohexyl)carbamate (3 g, 13.9 mmol) in DMF (60 mL) was added 1H-imidazole (2.37 g, 34.8 mmol) and TBSCl (2.52 g, 16.7 mmol), and the reaction was stirred for 16 h at rt. The resulting mixture was diluted with water (100 mL) and extracted with EtOAc (3×60 mL). The combined organic layers were washed with water (2×60 mL), dried over anhydrous Na₂SO₄, and filtered. The filtrate was concentrated under reduced pressure and purified by silica gel column chromatography (20% EtOAc in PE) to afford tert-butyl N-[4-[tert-butyl(dimethyl) silyl]oxycyclohexyl]carbamate (3 g, 9.1 mmol, 65% yield) as a solid. LCMS (ES, m/z): 330 [M+H]⁺.

### Synthesis of C18

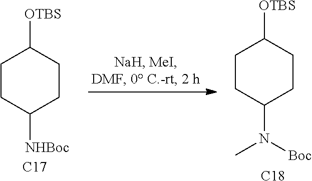 [SMILES: CC(C)(C)OC(=O)NC1CCC(O[Si](C)(C)C(C)(C)C)CC1 ; CN(C(=O)OC(C)(C)C)C1CCC(O[Si](C)(C)C(C)(C)C)CC1 ; "C17" ; "C18"]

To a mixture of tert-butyl N-[4-[tert-butyl(dimethyl) silyl]oxycyclohexyl]carbamate (2 g, 6.1 mmol) in DMF (40 mL) was added NaH (291.3 mg, 12.1 mmol), and the reaction was stirred for 30 minutes at rt. To the above mixture was added Mel (1.71 g, 12.1 mmol) in portions, and the reaction was stirred for an additional 2 h at room temperature. The resulting mixture was quenched by the addition of cold water (100 mL) and extracted with EtOAc (2×100 mL). The combined organic layers were washed with NH₄Cl (2×100 mL), dried over anhydrous Na₂SO₄, and filtered. The filtrate was concentrated under reduced pressure and purified by silica gel column chromatography (20% EtOAc in PE) to afford tert-butyl N-[4-[tert-butyl(dimethyl) silyl]oxycyclohexyl]-N-methyl-carbamate (1.2 g, 3.5 mmol, 58% yield) as a solid. LCMS (ES, m/z): 344 [M+H]⁺.

### Synthesis of C19

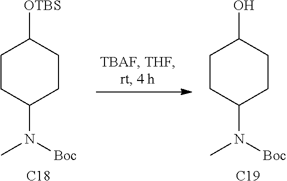 [SMILES: CN(C(=O)OC(C)(C)C)C1CCC(O[Si](C)(C)C(C)(C)C)CC1 ; CN(C(=O)OC(C)(C)C)C1CCC(O)CC1 ; "C18" ; "C19"]

To a stirred solution of tert-butyl N-[4-[tert-butyl(dimethyl) silyl]oxycyclohexyl]-N-methyl-carbamate (1.2 g, 3.5 mmol) in THF (24 mL) was added TBAF (1.5 g, 5.2 mmol), and the reaction was stirred for 4 h at rt. The resulting mixture was diluted with water (40 mL) and extracted with EtOAc (2×40 mL). The combined organic layer was washed with water (2×40 mL), dried by anhydrous Na₂SO₄, and filtered. The filtrate was concentrated under reduced pressure and purified by silica gel column chromatography (20% EtOAc in PE) to afford tert-butyl N-(4-hydroxycyclohexyl)-N-methyl-carbamate (0.6 g, 2.6 mmol, 75% yield) as a solid. LCMS (ES, m/z): 230 [M+H]⁺. ¹H NMR (300 MHz, DMSO-d₆) δ 4.60-4.40 (m, 1H), 3.3.90-3.50 (m, 1H), 2.61 (s, 3H), 1.89-1.73 (m, 2H), 1.55-1.42 (m, 2H), 1.37 (s, 9H), 1.25-1.17 (m, 2H).

An analogous method was followed to obtain the following intermediates.

| | Starting | |
|---|---|---|
| Intermediate | Material | Characterization |
| 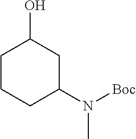 [SMILES: CN(C(=O)OC(C)(C)C)C1CCCC(O)C1] | 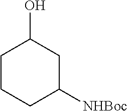 [SMILES: CC(C)(C)OC(=O)NC1CCCC(O)C1] | The residue was purified by silica gel column chromatography (20% EtOAc in PE) to afford tert-butyl N-(3- hydroxycyclohexyl)-N-methyl-carbamate (0.3 g, 1.3 mmol, 56% yield) as a solid. LCMS (ES, m/z): 230 [M + H]⁺. |
| C20 | | |
| 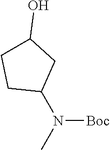 | 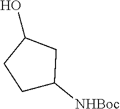 [SMILES: CC(C)(C)OC(=O)NC1CCC(O)C1] | The residue was purified by silica gel column chromatography (20% EtOAc in PE) to afford tert-butyl N-(3- hydroxycyclopentyl)-N-methyl-carbamate (0.2 g, 929 μmol, 77% yield) as a solid. LCMS (ES, m/z): 216 [M + H] . ¹H NMR (300 MHz, DMSO-d₆) δ 4.59 (d, J = 12.0 Hz, 1H), 4.50 (d, J = 3.5 Hz, 1H), 4.13 (dp, J = 5.3, 2.8, 2.3 Hz, 1H), 2.61 (s, 3H), 1.80 (tdd, J = 15.4, 8.3, 4.4 Hz, 2H), 1.59 (dt, J = 8.7, 4.5 Hz, 2H), 1.52-1.37 (m, 2H), 1.37 (s, 9H). |
| C21 | | |

### Example 79: Synthesis of C25

### Synthesis of C22

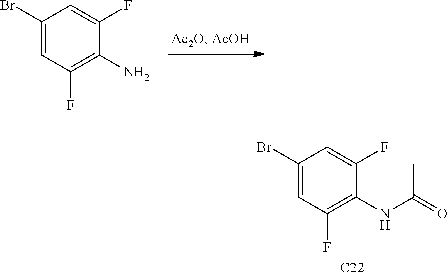

To a mixture of 4-bromo-2,6-difluoro-aniline (10 g, 48 mmol) in acetic acid (69 mL) was added acetic anhydride (33.3 g, 326 mmol, 31 mL). The reaction was stirred at 25° C. for 2 h. The reaction mixture was diluted with water at 0° C. (100 mL) and filtered. The filter cake was dissolved with DCM (50 mL), washed with saturated aqueous NaHCO₃ (50 mL) and brine (50 mL), dried over Na₂SO₄, and filtered. The filtrate was concentrated under reduced pressure to afford N-(4-bromo-2,6-difluoro-phenyl)acetamide (C22, 11.7 g, 88% yield) as a solid. The material was used in the next step without any purification. LCMS (ESI, m/z) 250.0 [M+H]⁺. ¹H NMR (400 MHz, CDCl₃) δ ppm 7.16 (br d, J=7.13 Hz, 2H), 6.75 (br s, 1H), 2.22 (br s, 3H).

### Synthesis of C23

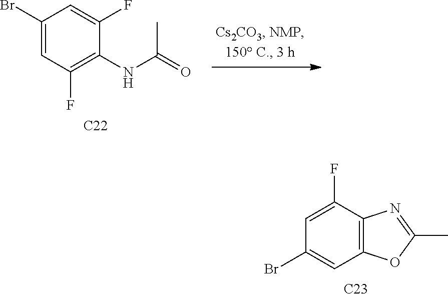

To a mixture of N-(4-bromo-2,6-difluoro-phenyl)acetamide (C22, 4 g, 16 mmol) in N-methyl-2-pyrrolidione (25 mL) was added Cs₂CO₃ (10.4 g, 31.9 mmol), and the reaction was stirred at 150° C. for 3 h. One additional vial was set up as described above and all two reaction mixtures were combined. The reaction mixture was poured into water (25 mL), extracted with EtOAc (3×20 mL), washed with brine (25 mL), dried over Na₂SO₄, and filtered. The filtrate was concentrated under reduced pressure and purified by silica gel column chromatography (4% EtOAc in PE) to afford 6-bromo-4-fluoro-2-methyl-1,3-benzoxazole (C23, 0.87 g, 21% yield) as an oil. LCMS (ESI, m/z): 230.0 [M+H]⁺. ¹H NMR (400 MHz, CDCl₃) δ ppm 7.48 (d, J=0.88 Hz, 1H), 7.22 (dd, J=9.07, 1.44 Hz, 1H), 2.63-2.66 (m, 3H).

### Synthesis of C24

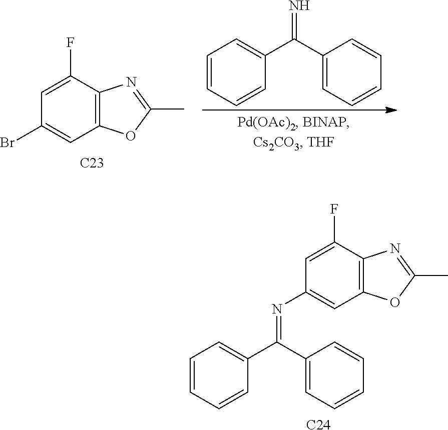

To a mixture of 6-bromo-4-fluoro-2-methyl-1,3-benzoxazole (C23, 0.67 g, 2.9 mmol) in THE (20 mL) was added diphenylmethanimine (686 mg, 3.8 mmol, 635 μL), Cs₂CO₃ (2.37 g, 7.3 mmol), Pd(OAc)₂ (65.3 mg, 291 μmol) and BINAP (181 mg, 291 μmol), and the reaction was stirred at 70° C. for 12 h. One additional vial was set up as described above and all two reaction mixtures were combined. The reaction mixture was diluted with water (20 mL), extracted with EtOAc (3×20 mL), washed with brine (40 mL), dried over Na₂SO₄, and filtered. The filtrate was concentrated under reduced pressure and purified by silica gel column chromatography (4% EtOAc in PE) to give N-(4-fluoro-2-methyl-1,3-benzoxazol-6-yl)-1,1-diphenyl-methanimine (C24, 1.1 g, 79% yield) as a solid. LCMS (ESI, m/z): 331.1 [M+H]⁺. ¹H NMR (400 MHz, CDCl₃) δ ppm 7.62-7.72 (m, 2H), 7.39-7.46 (m, 1H), 7.31-7.38 (m, 2H), 7.16-7.24 (m, 3H), 7.05 (dd, J=7.69, 1.56 Hz, 2H), 6.55 (d, J=1.50 Hz, 1H), 6.40 (dd, J=10.82, 1.56 Hz, 1H), 2.50 (s, 3H).

### Synthesis of C25

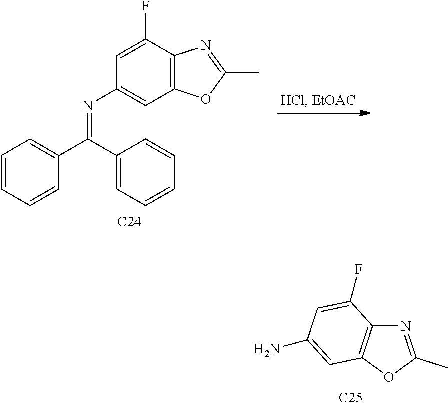 [SMILES: Cc1nc2c(F)cc(N=C(c3ccccc3)c3ccccc3)cc2o1 ; "C24" ; Cc1nc2c(F)cc(N)cc2o1 ; "C25"]

To a mixture of N-(4-fluoro-2-methyl-1,3-benzoxazol-6-yl)-1,1-diphenyl-methanimine (C24, 1.8 g, 5.4 mmol) in EtOAc (36 mL) was added HCl/EtOAc (4 M, 36 mL), and the reaction was stirred at 25° C. for 12 h. The reaction was filtered, and the filter cake was dried in vacuum to afford 4-fluoro-2-methyl-1,3-benzoxazol-6-amine (C25, 0.41 g, 82% yield) as a solid. The material was used in the next step without any purification. LCMS (ESI, m/z): 165.2 [M−H]⁻.

### Example 80: Synthesis of C27

### Synthesis of C26

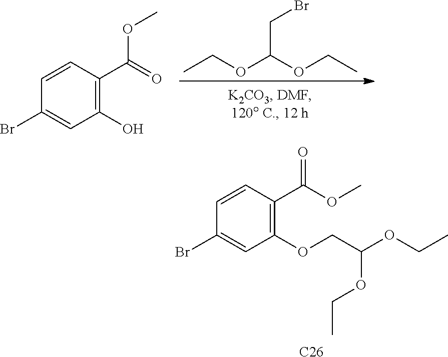

To a solution of methyl 4-bromo-2-hydroxy-benzoate (3 g, 13 mmol) and 2-bromo-1,1-diethoxy-ethane (3.8 g, 19.5 mmol) in dimethyl formamide (30 mL) was added K₂CO₃ (3.6 g, 26 mmol), and the reaction was stirred at 120° C. for 12 h. The mixture was poured into H₂O (15 mL) and extracted with DCM (3×50 mL). The combined organic phases were dried over Na₂SO₄, and filtered. The filtrate was concentrated under reduced pressure and purified by silica gel column chromatography (5-20% EtOAc in PE) to give methyl 4-bromo-2-(2,2-diethoxyethoxy)benzoate (C26, 3 g, 67% yield) as a solid. ¹H NMR (400 MHz, CDCl₃) δ ppm 7.67 (d, J=8.16 Hz, 1H), 7.11-7.18 (m, 2H), 4.87 (t, J=5.14 Hz, 1H), 4.06 (d, J=5.14 Hz, 2H), 3.87 (s, 3H), 3.75-3.84 (m, 2H), 3.67 (dq, J=9.27, 7.07 Hz, 2H), 1.25 (t, J=7.09 Hz, 6H).

### Synthesis of C27

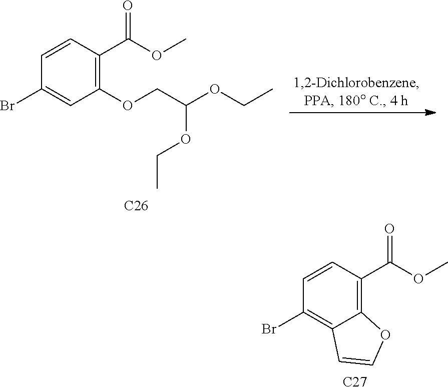 [SMILES: CCOC(COc1cc(Br)ccc1C(=O)OC)OCC ; "C26" ; COC(=O)c1ccc(Br)c2ccoc12 ; "C27"]

To a solution of methyl 4-bromo-2-(2,2-diethoxyethoxy)benzoate (C26, 1 g, 2.9 mmol) in 1,2-Dichlorobenzene (6 mL) was added polyphosphoric acid (2.26 g, 5.76 mmol) under N₂, and the reaction was stirred at 180° C. for 4 h. The resulting mixture was allowed to cool to room temperature and quenched by addition of saturated NaHCO₃ (80 mL). The aqueous phase was extracted with DCM (3×30 mL), and the combined organic phases were dried over Na₂SO₄, and filtered. The filtrate was concentrated under reduced pressure and purified by silica gel column chromatography (5-10% EtOAc in PE) to give methyl 4-bromobenzofuran-7-carboxylate (C27, 140 mg, 19% yield) as a solid. LCMS (ES, m/z): 255.0, 257.0 [M+H]⁺.

### Example 81: Synthesis of C33

### Synthesis of C28

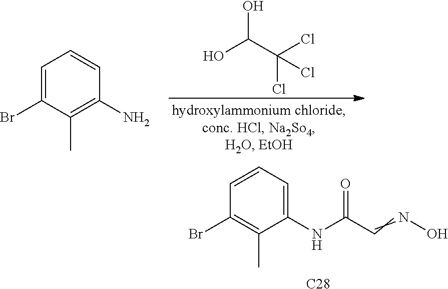

To a mixture of 2,2,2-trichloroethane-1,1-diol (80.0 g, 483 mmol, 1.5 eq), Na₂SO₄ (366 g, 2.58 mol, 8 eq) and hydroxylamine hydrochloride (78.4 g, 1.12 mol, 3.5 eq) in H₂O (1500 mL) was added a mixture of 3-bromo-2-methyl-aniline (60 g, 322 mmol, 1 eq) and HCl (12.0 M, 51 mL, 1.9 eq) in H₂O (1.62 L) and EtOH (200 mL) in portions, and the reaction was stirred at 60° C. for 12 h. The reaction was filtered, the filter cake was washed with water (2 L). The filter cake was dried under reduced pressure to afford N-(3-bromo-2-methylphenyl)-2-(hydroxyimino)-acetamide (C28, 80 g, 96% yield). LCMS (ESI, m/z): 257.1 [M+H]⁺. ¹H NMR (400 MHz, DMSO-d₆) δ ppm 12.22 (s, 1H), 9.81 (s, 1H), 7.66 (s, 1H), 7.49 (d, J=7.88 Hz, 1H), 7.40 (d, J=7.88 Hz, 1H), 7.15 (t, J=7.94 Hz, 1H), 2.25 (s, 3H).

### Synthesis of C29

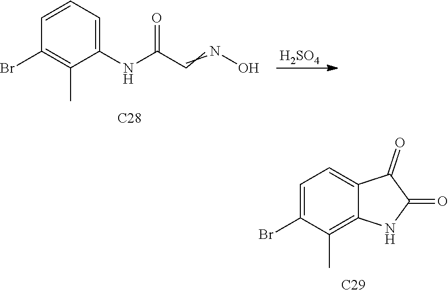

N-(3-bromo-2-methylphenyl)-2-(hydroxyimino)acetamide (C28, 80 g, 311 mmol) was added to sulfuric acid (640 mL) in portions at 25° C. The reaction mixture was stirred at 80° C. for 1 h. The mixture was poured into ice water (2 L) slowly and stirred for 10 mins, the resulting mixture was filtered, the filter cake was washed with water (2 L). The filter cake was dried under reduced pressure to afford 6-bromo-7-methylindoline-2,3-dione (C29, 65.4 g, 88% yield) as a solid. LCMS (ESI, m/z): 239.9 [M+H]⁺. ¹H NMR (400 MHz, DMSO-d₆) δ ppm 11.26 (br s, 1H), 7.30-7.36 (m, 1H), 7.23-7.29 (m, 1H), 2.24 (s, 3H).

### Synthesis of C30

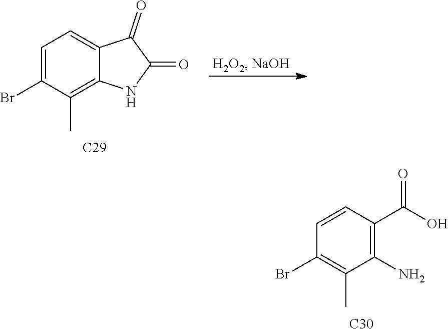

To a mixture of 6-bromo-7-methylindoline-2,3-dione (C29, 32.5 g, 135 mmol, 1 eq) in NaOH (2 M, 609 mL, 9 eq) was added hydrogen peroxide (23 g, 676 mmol, 20.7 mL, 5 eq) dropwise at 25° C. (over 15 mins), and the reaction was stirred at 25° C. for 2 h. The reaction was quenched with saturated aqueous sodium sulfite (100 mL) at 25° C. The mixture was acidified pH=6 by addition of aqueous HCl (2 M, 800 mL) and filtered. The filter cake was washed with water (500 mL). The filter cake was dried under reduced pressure to afford 2-amino-4-bromo-3-methylbenzoic acid (C30, 30 g, 88% yield) as a solid. LCMS (ESI, m/z): 230.5 [M+H]⁺. ¹H NMR (400 MHz, DMSO-d₆) δ ppm 7.51 (d, J=8.63 Hz, 1H), 6.78 (d, J=8.63 Hz, 1H), 2.22 (s, 3H).

### Synthesis of C31

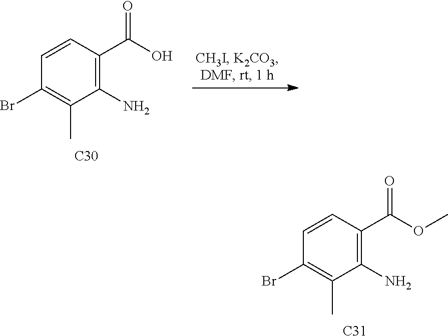

To a suspension of 2-amino-4-bromo-3-methylbenzoic acid (C30, 29.5 g, 128 mmol, 1 eq) and potassium carbonate (26.5 g, 192 mmol, 1.50 eq) in dimethyl formamide (511 mL) was added trideuterio (iodo) methane (22.3 g, 153 mmol, 9.6 mL) dropwise at 0° C. under N₂. The mixture was stirred at 25° C. for 1 h. The mixture was poured into water (1 L), then extracted with EtOAc (3×500 mL). The combined organic phase was washed with brine (1 L), dried over Na₂SO₄, and filtered. The filtrate was concentrated under reduced pressure to afford methyl 2-amino-4-bromo-3-methylbenzoate (C31, 27 g, 86% yield) as a solid. LCMS (ESI, m/z): 244.5 [M+H]⁺. ¹H NMR (400 MHz, DMSO-d₆) δ ppm 7.52 (d, J=8.75 Hz, 1H), 6.84 (br s, 2H), 6.81 (d, J=8.76 Hz, 1H), 3.79 (s, 3H), 2.23 (s, 3H).

### Synthesis of C32

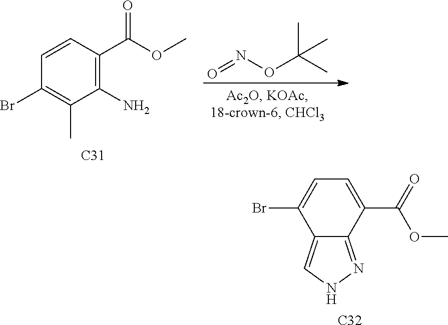

To a mixture of 2-amino-4-bromo-3-methylbenzoate (C31, 30 g, 122 mmol, 1 eq) and potassiumacetate (13.2 g, 135 mmol, 1.1 eq) in trichloromethane (626 mL) was added acetyl acetate (25 g, 245 mmol, 23.1 mL, 2 eq) dropwise at 0° C., and the reaction was stirred for 20 min at 25° C. Then 1,4,7,10,13,16-hexaoxacyclooctadecane (5.85 g, 22.1 mmol, 0.18 eq) and tert-butyl nitrite (27.8 g, 270 mmol, 2.2 eq) was added to the reaction mixture successively, and the reaction was stirred at 65° C. for 2 h. The mixture was diluted with saturated aqueous NaHCO₃ (200 mL) and extracted with DCM (2×300 mL). The combined organic layers were washed with brine (1 L), dried over Na₂SO₄ and concentrated under reduced pressure to afford methyl 4-bromo-2H-indazole-7-carboxylate (C32, 25 g, 80% yield) as a solid. LCMS (ESI, m/z): 255.1 [M+H]⁺. ¹H NMR (400 MHz, DMSO-d₆) δ ppm 13.61 (br s, 1H), 8.22 (s, 1H), 7.91 (d, J=7.75 Hz, 1H), 7.51 (d, J=7.88 Hz, 1H), 3.96 (s, 3H).

### Synthesis of C33

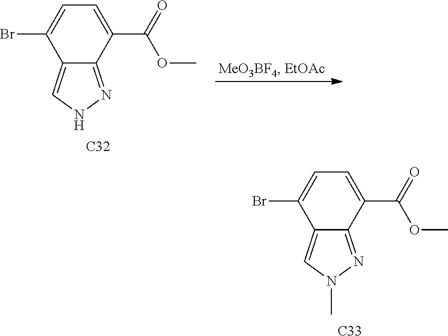

To a mixture of methyl 4-bromo-2H-indazole-7-carboxylate (C32, 25 g, 98 mmol) in EtOAc (490 mL) was added trimethyloxonium tetrafluoroborate (21.7 g, 147 mmol, 1.5 eq) in portions, and the reaction was stirred at 25° C. for 12 h. The mixture was washed with brine (400 mL), dried over Na₂SO₄ and filtered. The filtrate was concentrated under pressure and triturated with MTBE (150 mL) to afford methyl 4-bromo-2-methyl-2H-indazole-7-carboxylate (C33, 23 g, 87% yield) as a solid. LCMS (ESI, m/z): 269.6 [M+H]⁺. ¹H NMR (400 MHz, DMSO-d₆) δ ppm 8.61 (s, 1H), 7.83 (d, J=7.63 Hz, 1H), 7.40 (d, J=7.63 Hz, 1H), 4.24 (s, 3H), 3.88 (s, 3H).

### Example 82: Synthesis of Compound 193

### Synthesis of C34

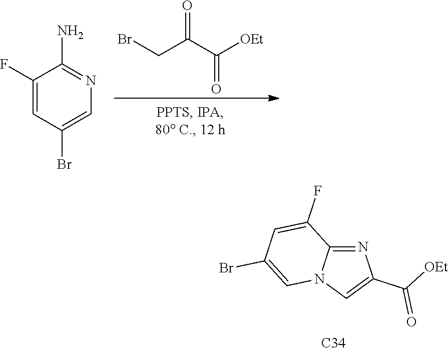 [SMILES: Nc1ncc(Br)cc1F ; CCOC(=O)C(=O)CBr ; CCOC(=O)c1cn2cc(Br)cc(F)c2n1 ; "C34"]

To a solution of 5-bromo-3-fluoro-pyridin-2-amine (500 mg, 2.6 mmol) and ethyl 3-bromo-2-oxo-propanoate (561 mg, 2.88 mmol, 361 μL) in isopropanol (6.3 mL) was added 4-methylbenzenesulfonic acid pyridine (65.7 mg, 288 μmol) under N₂, and the reaction was stirred at 80° C. for 12 h. The reaction mixture was poured into water (20 mL) and extracted with EtOAc (3×20 mL). The combined organic phase was washed with brine (20 mL), dried over anhydrous Na₂SO₄, and filtered. The filtrate was concentrated under reduced pressure and purified by silica gel column chromatography (11% EtOAc in PE) to give ethyl 6-bromo-8-fluoro-imidazo[1,2-a]pyridine-2-carboxylate (C34, 250 mg, 33% yield) as a solid. LCMS (ES, m/z): 287.1, 289.1 [M+H]⁺. ¹H NMR (400 MHz, Methanol-d₄) δ ppm 8.64 (s, 1H), 8.49 (d, J=2.88 Hz, 1H), 7.41 (dd, J=10.01, 1.25 Hz, 1H), 4.41 (q, J=7.13 Hz, 2H), 1.41 (t, J=7.13 Hz, 3H).

### Synthesis of C35

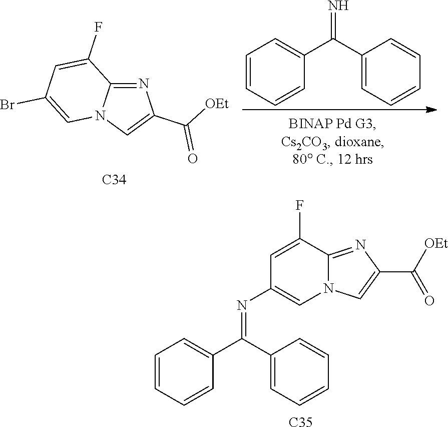

To a solution of ethyl 6-bromo-8-fluoro-imidazo[1,2-a]pyridine-2-carboxylate (C34, 2 g, 6.97 mmol) and diphenylmethanimine (1.52 g, 8.36 mmol) in dioxane (20 mL) was added BINAP Pd G3 (207 mg, 209 μmol) and Cs₂CO₃ (3.4 g, 10.5 mmol) under N₂, and the reaction was stirred at 80° C. for 12 h. Four additional vials were set up as described above and five reaction mixtures were combined. The reaction mixture was poured into water (20 mL) and extracted with EtOAc (2×50 mL). The combined organic layers were washed with brine (100 mL) and dried over Na₂SO₄, and filtered. The filtrate was concentrated under reduced pressure and purified by silica gel chromatography (8% EtOAc in PE) to afford ethyl 6-(benzhydrylideneamino)-8-fluoro-imidazo[1,2-a]pyridine-2-carboxylate (C35, 6 g, 52% yield) as a solid. LCMS (ES, m/z): 388.5 [M+H]⁺. ¹H NMR (400 MHz, DMSO-d₆) δ ppm 8.79 (s, 1H), 8.59 (br s, 1H), 8.50 (br s, 1H), 7.92 (s, 1H), 7.64-7.74 (m, 2H), 7.56-7.63 (m, 1H), 7.46-7.54 (m, 2H), 7.38 (br s, 2H), 7.22-7.29 (m, 1H), 6.92 (br d, J=12.01 Hz, 1H), 4.26-4.36 (m, 2H), 1.27-1.35 (m, 3H).

### Synthesis of C36

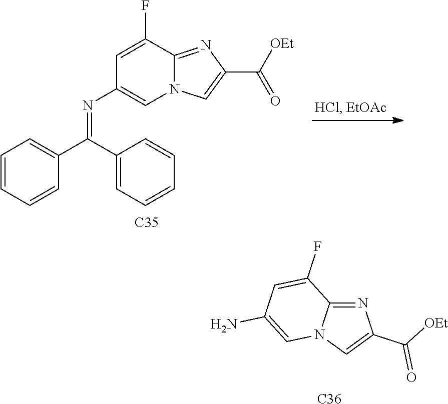 [SMILES: CCOC(=O)c1cn2cc(N=C(c3ccccc3)c3ccccc3)cc(F)c2n1 ; "C35" ; CCOC(=O)c1cn2cc(N)cc(F)c2n1 ; "C36"]

To a solution of ethyl 6-(benzhydrylideneamino)-8-fluoro-imidazo[1,2-a]pyridine-2-carboxylate (C35, 4 g, 10.3 mmol) in EtOAc (40 mL) was added HCl/EtOAc (4 M, 40 mL), and the reaction was stirred at 25° C. for 2 h. The reaction mixture was concentrated under reduced pressure, poured into water (100 mL), and extracted with MTBE (3×100 mL). The aqueous phase was acidified to pH=7 with saturated NaHCO₃ and the resulting mixture was extracted with EtOAc (3×20 mL). The combined organic phase was washed with brine (20 mL), dried over anhydrous Na₂SO₄, filtered, and concentrated to give ethyl 6-amino-8-fluoro-imidazo[1,2-a]pyridine-2-carboxylate (C36, 2 g, 87% yield) as a solid. LCMS (ES, m/z): 224.2 [M+H]⁺. ¹H NMR (400 MHz, Methanol-d₄) δ ppm 8.23 (d, J=2.76 Hz, 1H), 7.59 (d, J=1.63 Hz, 1H), 6.86 (dd, J=12.30, 1.76 Hz, 1H), 4.37 (q, J=7.15 Hz, 2H), 1.39 (t, J=7.09 Hz, 3H). ¹⁹F NMR (376 MHz, Methanol-d₄) δ ppm −132.66 (s, 1F).

### Synthesis of B44

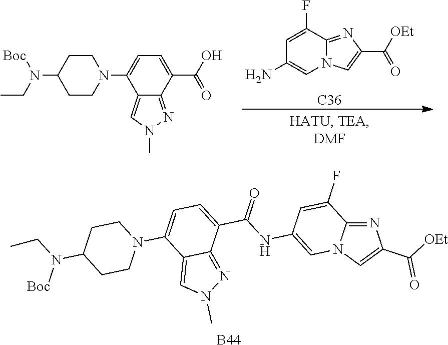 [SMILES: "C36" ; CCN(C(=O)OC(C)(C)C)C1CCN(c2ccc(C(=O)O)c3nn(C)cc23)CC1 ; CCOC(=O)c1cn2cc(N)cc(F)c2n1 ; CCOC(=O)c1cn2cc(NC(=O)c3ccc(N4CCC(N(CC)C(=O)OC(C)(C)C)CC4)c4cn(C)nc34)cc(F)c2n1 ; "B44"]

To a solution of ethyl 6-amino-8-fluoro-imidazo[1,2-a]pyridine-2-carboxylate (C36, 500 mg, 2.24 mmol) and 4-[4-[tert-butoxycarbonyl(ethyl)amino]-1-piperidyl]-2-methyl-indazole-7-carboxylic acid (650 mg, 1.6 mmol) in dimethyl formamide (2.5 mL) was added triethylamine (490 mg, 4.84 mmol, 675 μL) and o-(7-azabenzotriazol-1-yl)-n,n,n,n-tetramethyluronium hexafluorophosphate (921 mg, 2.42 mmol), and the reaction was stirred at 50° C. for 12 h. The reaction mixture was poured into water (20 mL) and extracted with DCM (3×20 mL). The combined organic phase was washed with brine (20 mL), dried with anhydrous Na₂SO₄, and filtered. The filtrate was concentrated under reduced pressure and purified by silica gel column chromatography (50% EtOAc in PE) to give ethyl 6-[[4-[4-[tert-butoxycarbonyl(ethyl)amino]-1-piperidyl]-2-methyl-indazole-7-carbonyl]amino]-8-fluoro-imidazo[1,2-a]pyridine-2-carboxylate (B44, 500 mg, 51% yield) as a solid. LCMS (ES, m/z): 608.5 [M+H]⁺. ¹H NMR (400 MHz, CDCl₃) δ ppm 11.14 (s, 1H), 9.37 (s, 1H), 8.22-8.24 (m, 1H), 8.04 (s, 1H), 6.99 (dd, J=10.88, 1.38 Hz, 1H), 6.52 (d, J=8.00 Hz, 1H), 4.46-4.52 (m, 2H), 4.46 (s, 1H), 4.31 (s, 3H), 3.97 (br d, J=12.63 Hz, 2H), 3.20 (br d, J=4.75 Hz, 2H), 3.01-3.10 (m, 2H), 1.86-1.93 (m, 3H), 1.50 (s, 9H), 1.42-1.48 (m, 4H), 1.16 (t, J=7.00 Hz, 3H).

### Synthesis of B45

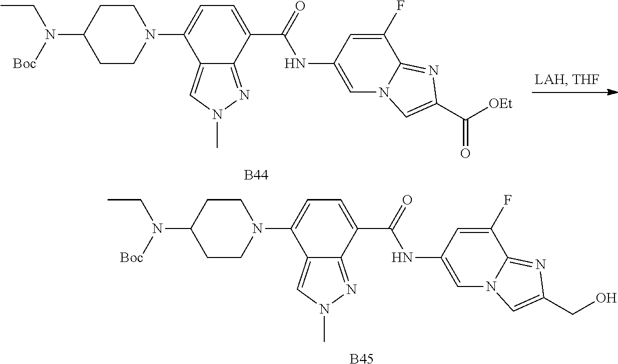

To a solution of ethyl 6-[[4-[4-[tert-butoxycarbonyl(ethyl)amino]-1-piperidyl]-2-methyl-indazole-7-carbonyl]amino]-8-fluoro-imidazo[1,2-a]pyridine-2-carboxylate (B44, 500 mg, 822 μmol) in THF (5 mL) was added LiAlH₄ (2.5 M, 427 μL) at 0° C. under N₂. The mixture was stirred at 0° C. for 1 h. The reaction mixture was filtered to remove insoluble solids. The filtrate was concentrated under reduced pressure and purified by silica gel column chromatography (50% EtOAc in PE) to give tert-butyl N-ethyl-N-[1-[7-[[8-fluoro-2-(hydroxymethyl)-imidazo[1,2-a]pyridin-6-yl]carbamoyl]-2-methyl-indazol-4-yl]-4-piperidyl]carbamate (B45, 400 mg, 86% yield) as a solid. LCMS (ES, m/z): 566.5 [M+H]⁺. ¹H NMR (400 MHz, Methanol-d₄) δ ppm 8.98 (s, 1H), 8.33 (s, 1H), 7.96 (d, J=8.00 Hz, 1H), 7.77 (d, J=2.38 Hz, 1H), 7.06 (br d, J=11.63 Hz, 1H), 6.40 (d, J=8.13 Hz, 1H), 4.73 (s, 2H), 4.24 (s, 3H), 3.98 (br d, J=12.26 Hz, 3H), 3.24 (br d, J=6.75 Hz, 2H), 2.94 (br t, J=12.07 Hz, 2H), 1.92-2.02 (m, 2H), 1.80 (br d, J=10.76 Hz, 2H), 1.48 (s, 9H), 1.16 (t, J=6.94 Hz, 3H).

### Synthesis of Compound 193

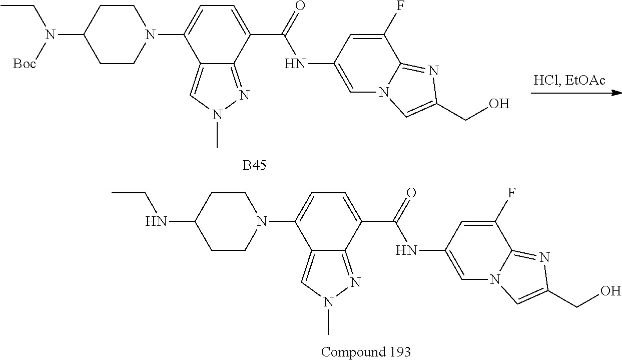

To a solution of tert-butyl N-ethyl-N-[1-[7-[[8-fluoro-2-(hydroxymethyl) imidazo[1,2-a]-pyridin-6-yl]carbamoyl]-2-methyl-indazol-4-yl]-4-piperidyl]carbamate (B45, 60 mg, 106 μmol) in EtOAc (1.2 mL) was added HCl/EtOAc (4 M, 1.2 mL), and the reaction was stirred at 25° C. for 2 h. The mixture was concentrated under reduced pressure and triturated with DCM (3 mL). The mixture was filtered and the filter cake was concentrated under reduced pressure to afford 4-[4-(ethylamino)-1-piperidyl]-N-[8-fluoro-2-(hydroxymethyl) imidazo[1,2-a]pyridin-6-yl]-2-methyl-indazole-7-carboxamide (Compound 193, 26 mg, 53%, as HCl salt) as a solid. LCMS (ES, m/z): 466.3 [M+H]⁺. ¹H NMR (400 MHz, DMSO-d₆) δ ppm 11.30 (s, 1H), 9.57 (s, 1H), 9.07 (br d, J=4.13 Hz, 2H), 8.88 (s, 1H), 8.33 (s, 1H), 7.91-8.04 (m, 2H), 6.55 (d, J=8.25 Hz, 1H), 4.70 (s, 2H), 4.31 (s, 3H), 4.06 (br d, J=13.26 Hz, 2H), 3.27-3.35 (m, 1H), 3.02 (br d, J=12.88 Hz, 4H), 2.15-2.21 (m, 2H), 1.74-1.84 (m, 2H), 1.26 (t, J=7.19 Hz, 3H). ¹⁹F NMR (376 MHz, DMSO-d₆) δ ppm −131.51 (s, 1F).

### Example 83: Synthesis of Compound 261

### Synthesis of B46

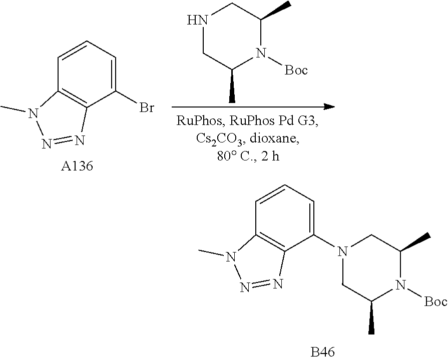

To a mixture of 4-bromo-1-methyl-benzotriazole (200 mg, 943 μmol) and tert-butyl (2R,6S)-2,6-dimethylpiperazine-1-carboxylate (243 mg, 1.13 mmol) in Dioxane (5 mL) were added Cs₂CO₃ (615 mg, 1.9 mmol), RuPhos (88 mg, 188.6 μmol) and RuPhos Pd G3 (79 mg, 94.3 μmol), and the reaction was stirred for 2 h at 80° C. under N₂. The resulting mixture was concentrated under reduced pressure and purified by silica gel column chromatography (67% EtOAc in PE) to afford tert-butyl (2R,6S)-2,6-dimethyl-4-(1-methylbenzotriazol-4-yl)piperazine-1-carboxylate (200 mg, 579 μmol, 61% yield) as a solid. LCMS (ES, m/z): 346 [M+H]⁺.

### Synthesis of B47

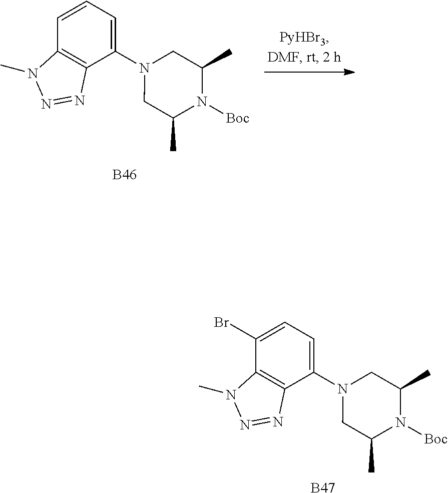 [SMILES: "B46" ; C[C@@H]1CN(c2cccc3c2nnn3C)C[C@H](C)N1C(=O)OC(C)(C)C ; "B47" ; C[C@@H]1CN(c2ccc(Br)c3c2nnn3C)C[C@H](C)N1C(=O)OC(C)(C)C]

To a stirred solution of tert-butyl (2R,6S)-2,6-dimethyl-4-(1-methylbenzotriazol-4-yl)-piperazine-1-carboxylate (200 mg, 579 μmol) in DMF (5 mL) was added 3,4,5-tribromopyridine (183 mg, 579 μmol), and the reaction was stirred for 2 h at rt. The resulting mixture was diluted with water (20 mL) and extracted with EtOAc (2×20 mL). The combined organic layers were washed with brine (2×20 mL), dried over anhydrous Na₂SO₄, and filtered. The filtrate was concentrated under reduced pressure and purified by silica gel column chromatography (33% EtOAc in DCM) to afford tert-butyl (2R,6S)-4-(7-bromo-1-methyl-benzotriazol-4-yl)-2,6-dimethyl-piperazine-1-carboxylate (B47, 200 mg, 471 μmol, 81% yield) as a solid. LCMS (ES, m/z): 424 [M+H]⁺.

### Synthesis of B48

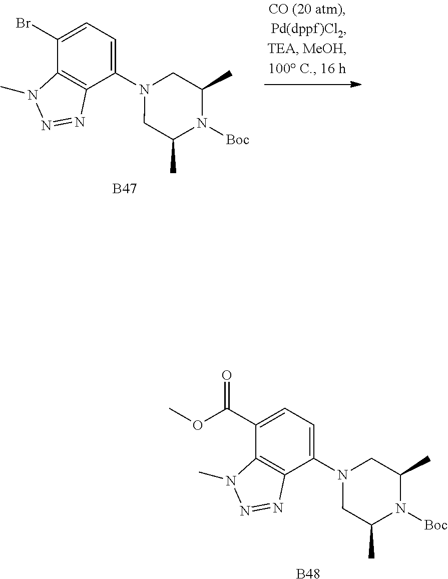

To a stirred mixture of tert-butyl (2R,6S)-4-(7-bromo-1-methyl-benzotriazol-4-yl)-2,6-dimethyl-piperazine-1-carboxylate (200 mg, 471.3 μmol) in MeOH (5 mL) were added Pd(dppf)Cl₂ (34 mg, 47.13 μmol) and TEA (95 mg, 942.7 μmol, 131.4 μL), and the reaction was stirred 90° C. for 16 h under carbon monoxide atmosphere (20 atm). The solid was filtered out and the filtrate was concentrated under reduced pressure and purified by silica gel column chromatography (67% EtOAc in PE) to afford methyl 7-[(3R,5S)-4-tert-butoxycarbonyl-3,5-dimethyl-piperazin-1-yl]-3-methyl-benzotriazole-4-carboxylate (140 mg, 347 μmol, 74% yield) as an oil. LCMS (ES, m/z): 404 [M+H]⁺.

### Synthesis of B49

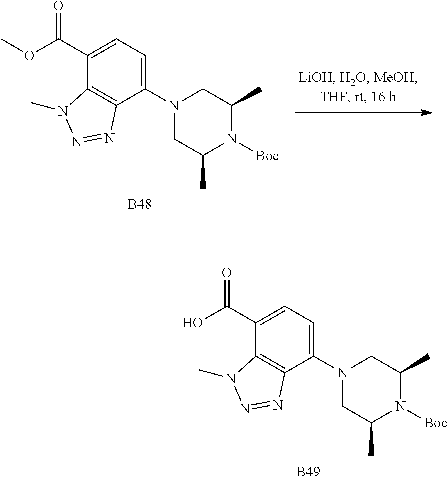

To a stirred mixture of methyl 7-[(3R,5S)-4-tert-butoxycarbonyl-3,5-dimethyl-piperazin-1-yl]-3-methyl-benzotriazole-4-carboxylate (160 mg, 396.6 μmol) in THF (3 mL), H₂O (3 mL) and MeOH (3 mL) was added LiOH (47.4 mg, 1.98 mmol), and the reaction was stirred for 16 h at room temperature. The resulting mixture was diluted with water (50 mL) and neutralized to pH 5 with HCl (aq.). The aqueous layer was extracted with EtOAc (2×50 mL). The combined organic layer was washed with brine (30 mL) and dried by anhydrous Na₂SO₄, and filtered. The filtrate was concentrated under reduced pressure to afford 7-[(3R,5S)-4-tert-butoxycarbonyl-3,5-dimethyl-piperazin-1-yl]-3-methyl-benzotriazole-4-carboxylic acid (140 mg, 359.5 μmol, 91% yield) as a solid. LCMS (ES, m/z): 390 [M+H]⁺.

### Synthesis of B50

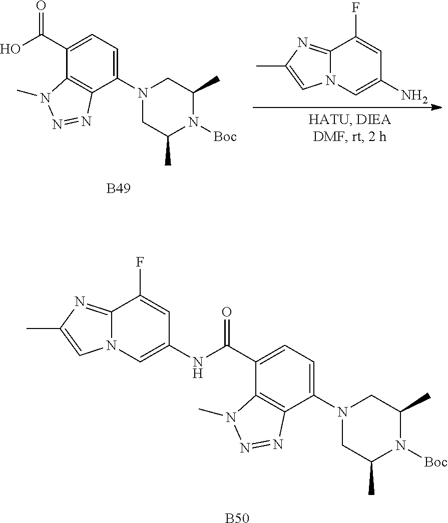

To a stirred mixture of 7-[(3R,5S)-4-tert-butoxycarbonyl-3,5-dimethyl-piperazin-1-yl]-3-methyl-benzotriazole-4-carboxylic acid (140 mg, 359.5 μmol) and 8-fluoro-2-methyl-imidazo[1,2-a]pyridin-6-amine (71 mg, 431.4 μmol) in DMF (5 mL) were added DIEA (139 mg, 1.08 mmol, 187.8 μL) and HATU (410 mg, 1.08 mmol), and the reaction was stirred for 2 h at room temperature. The resulting mixture was extracted with EtOAc (10 mL). The combined organic layers were washed with water (2×100 mL), dried over anhydrous Na₂SO₄, and filtered. The filtrate was concentrated under reduced pressure and purified by silica gel column chromatography (67% EtOAc in PE) to afford tert-butyl (2R,6S)-4-[7-[(8-fluoro-2-methyl-imidazo[1,2-a]pyridin-6-yl)carbamoyl]-1-methyl-benzotriazol-4-yl]-2,6-dimethyl-piperazine-1-carboxylate (B50, 100 mg, 186.4 μmol, 52% yield) as a solid. LCMS (ES, m/z): 537 [M+H]⁺.

### Synthesis of Compound 261

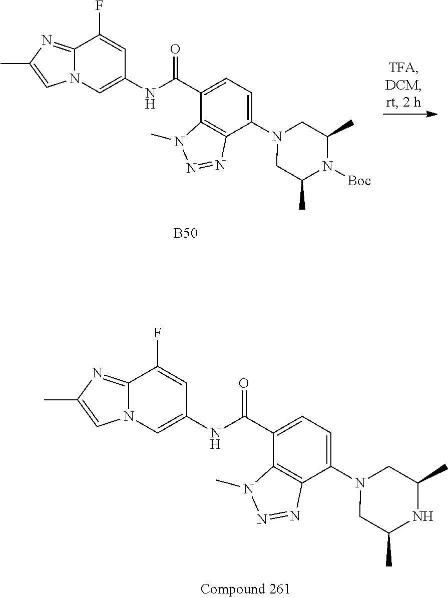

To a stirred solution of tert-butyl (2R,6S)-4-[7-[(8-fluoro-2-methyl-imidazo[1,2-a]-pyridin-6-yl)carbamoyl]-1-methyl-benzotriazol-4-yl]-2,6-dimethyl-piperazine-1-carboxylate (B50, 100 mg, 186.4 μmol) in DCM (2 mL) was added TFA (1.5 g, 13.1 mmol, 1 mL), and the reaction was stirred for 2 h at room temperature. The resulting mixture was concentrated under reduced pressure and purified by Prep-HPLC (Condition 6, Gradient 2) to afford 7-[(3R,5S)-3,5-dimethylpiperazin-1-yl]-N-(8-fluoro-2-methyl-imidazo[1,2-a]pyridin-6-yl)-3-methyl-benzotriazole-4-carboxamide (Compound 261, 26.6 mg, 61 μmol, 33% yield) as a solid. LCMS (ES, m/z): 437 [M+H]⁺. ¹H NMR (400 MHz, DMSO-d₆) δ 10.52 (s, 1H), 9.13 (d, J=1.6 Hz, 1H), 7.94 (d, J=3.1 Hz, 1H), 7.73 (d, J=8.2 Hz, 1H), 7.28 (dd, J=12.7, 1.6 Hz, 1H), 6.68 (d, J=8.4 Hz, 1H), 4.65-4.57 (m, 2H), 4.30 (s, 3H), 2.95 (ddd, J=9.6, 6.4, 2.9 Hz, 2H), 2.59-2.48 (m, 17H), 2.35 (s, 3H), 1.07 (d, J=6.2 Hz, 6H).

An analogous method was followed to obtain the following compounds.

| | Starting | |
|---|---|---|
| Compound | Material | Characterization |
| 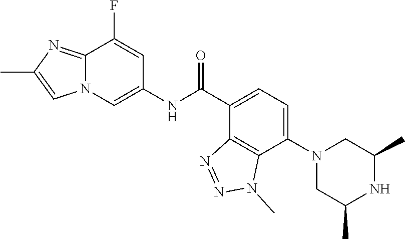 | 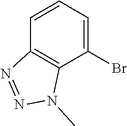 | The residue was purified by reversed-phase flash chromatography (Condition 3, Gradient 9) to afford 7-[(3R,5S)-3,5-dimethyl-piperazin- 1-y1]-N-(8-fluoro-2-methylimidazo[1,2- alpyridin-6-yl)-1-methyl-benzotriazole-4- carboxamide (Compound 635, 0.024 g, 55 μmol, 42% yield) as a solid. LCMS (ES, m/z): 437 [M + H]⁺. ¹H NMR (300 MHz, DMSO-d₆) δ 10.88 (s, 1H), 9.18 (d, J = 1.7 Hz, 1H), 8.00 (d, J = 7.9 Hz, 1H), 7.93 (dd, J = 3.3, 1.0 Hz, 1H), 7.40 (dd, J = 12.4, 1.7 Hz, 1H), 7.22 (d, J = 8.0 Hz, 1H), 4.56 (s, 3H), 3.25 (d, J = 11.0 Hz, 2H), 3.07 (s, 2H), 2.43-2.30 (m, 5H), 1.05 (d, J = 6.3 Hz, 6H). |
| Compound 635 | | |

### Example 84: Synthesis of Compound 606

### Synthesis of B51

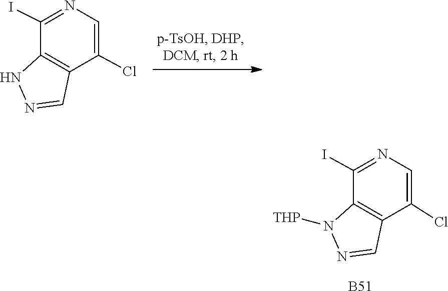

To a stirred mixture of 4-chloro-7-iodo-1H-pyrazolo[3,4-c]pyridine (350 mg, 1.25 mmol), p-TsOH (43 mg, 250.5 μmol) in DCM (7 mL) was added DHP (90 mg, 2.5 mmol), and the reaction was stirred for 2 h at 60° C. The resulting mixture was diluted with water (20 mL) and extracted with DCM (2×20 mL). The combined organic layers were washed with brine (20 mL), dried over anhydrous Na₂SO₄, and filtered. The filtrate was concentrated under reduced pressure and purified by silica gel column chromatography (50% EtOAc in PE) to afford 4-chloro-7-iodo-1-tetrahydropyran-2-yl-pyrazolo[3,4-c]pyridine (B51, 380 mg, 1.05 mmol, 84% yield) as a solid. LCMS (ES, m/z): 364 [M+H]⁺.

### Synthesis of B52

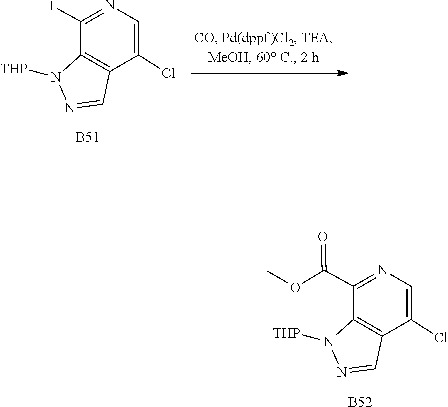

To a solution of 4-chloro-7-iodo-1-tetrahydropyran-2-yl-pyrazolo[3,4-c]pyridine (B51, 370 mg, 1.02 mmol) in 20 mL of MeOH was added Pd(dppf)Cl₂ (74 mg, 101.8 μmol) and TEA (412 mg, 4.07 mmol, 567.4 μL) in a pressure tank. The mixture was purged with N₂ and then was pressurized to 2 Mpa with carbon monoxide at 60° C. for 2 h. The reaction mixture was cooled to room temperature and filtered to remove insoluble solid, to afford methyl 4-chloro-1-tetrahydropyran-2-yl-pyrazolo[3,4-c]pyridine-7-carboxylate (B52, 290 mg, 980.7 μmol, 96% yield) as a solid. LCMS (ES, m/z): 296 [M+H]⁺.

### Synthesis of B53

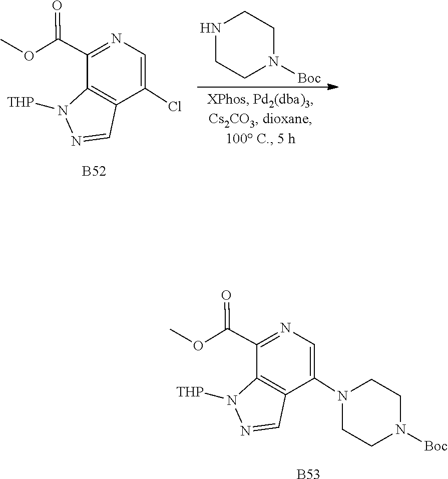

To a solution of methyl 4-chloro-1-tetrahydropyran-2-yl-pyrazolo[3,4-c]pyridine-7-carboxylate (B52, 280 mg, 946.8 μmol) and tert-butyl piperazine-1-carboxylate (265 mg, 1.42 mmol) in dioxane (5 mL) were added Cs₂CO₃ (617 mg, 1.89 mmol) and Pd₂(dba)₃ (87 mg, 94.7 μmol), XPhos (90 mg, 189.4 μmol), and the reaction was stirred for 5 h at 100° C. under N₂. The resulting mixture was concentrated under reduced pressure and purified by silica gel column chromatography (67% EtOAc in PE) to afford methyl 4-(4-tert-butoxycarbonylpiperazin-1-yl)-1-tetrahydropyran-2-yl-pyrazolo[3,4-c]pyridine-7-carboxylate (B53, 300 mg, 673.4 μmol, 71% yield) as a solid. LCMS (ES, m/z): 446 [M+H]⁺.

### Synthesis of B54

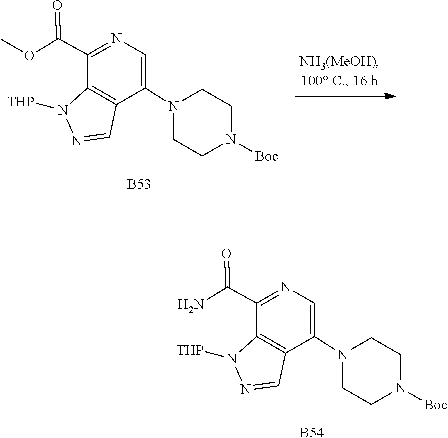

Into a 40 mL pressure tank reactor were added methyl 4-(4-tert-butoxycarbonylpiperazin-1-yl)-1-tetrahydropyran-2-yl-pyrazolo[3,4-c]pyridine-7-carboxylate (B53, 270 mg, 606 μmol) and NH₃ (7 M in MeOH) (6 mL), and the reaction was stirred for 16 h at 100° C. The resulting mixture was concentrated under reduced pressure and purified by silica gel column chromatography (67% EtOAc in PE) to afford tert-butyl 4-(7-carbamoyl-1-tetrahydropyran-2-yl-pyrazolo[3,4-c]pyridin-4-yl)piperazine-1-carboxylate (B54, 250 mg, 580.7 μmol, 96% yield) as a solid. LCMS (ES, m/z): 431 [M+H]⁺.

### Synthesis of B55

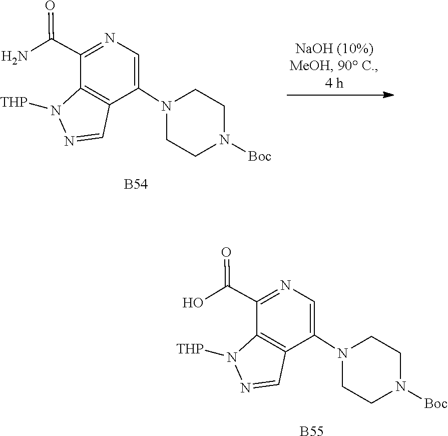

To a stirred mixture of tert-butyl 4-(7-carbamoyl-1-tetrahydropyran-2-yl-pyrazolo[3,4-c]-pyridin-4-yl)piperazine-1-carboxylate (B54, 200 mg, 464.6 μmol) in MeOH (2 mL) and H₂O (2 mL) was added NaOH (372 mg, 9.3 mmol, 174.5 μL), and the reaction was stirred for 4 h at 90° C. under N₂. The resulting mixture was diluted with water (5 mL), acidified to pH 6 with HCl (2N), and extracted with EtOAc (10 mL). The combined organic layers were washed with H₂O (10 mL), dried over anhydrous Na₂SO₄, filtered, and concentrated under reduced pressure to afford 4-(4-tert-butoxycarbonylpiperazin-1-yl)-1-tetrahydropyran-2-yl-pyrazolo[3,4-c]pyridine-7-carboxylic acid (B55, 150 mg, 347.6 μmol, 75% yield) as a solid. LCMS (ES, m/z): 432 [M+H]⁺.

### Synthesis of B56

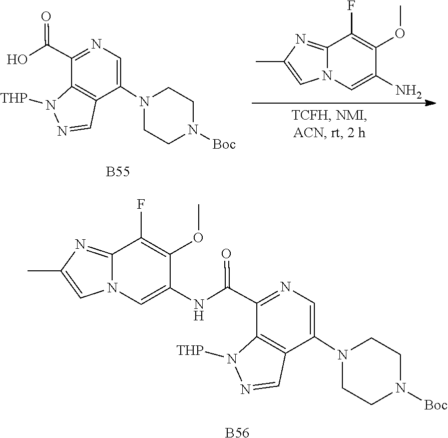

To a stirred mixture of 4-(4-tert-butoxycarbonylpiperazin-1-yl)-1-tetrahydropyran-2-yl-pyrazolo[3,4-c]pyridine-7-carboxylic acid (B55, 100 mg, 231.8 μmol) and 8-fluoro-7-methoxy-2-methyl-imidazo[1,2-a]pyridin-6-amine (54 mg, 278.1 μmol) in MeCN (2 mL) was added NMI (76 mg, 927 μmol, 73.5 μL) and TCFH (91 mg, 324.5 μmol), and the reaction was stirred for 2 h at 25° C. The reaction was quenched with water (10 mL) and extracted with EtOAc (2×10 mL). The combined organic layers were washed with brine (10 mL), dried over anhydrous Na₂SO₄, and filtered. The filtrate was concentrated under reduced pressure and purified by silica gel column chromatography (5% MeOH in DCM) to afford tert-butyl 4-[7-[(8-fluoro-7-methoxy-2-methylimidazo[1,2-a]pyridin-6-yl)carbamoyl]-1-tetrahydropyran-2-yl-pyrazolo[3,4-c]pyridin-4-yl]piperazine-1-carboxylate (B56, 75 mg, 123 μmol, 53% yield) as a solid. LCMS (ES, m/z): 609 [M+H]⁺.

### Synthesis of Compound 606

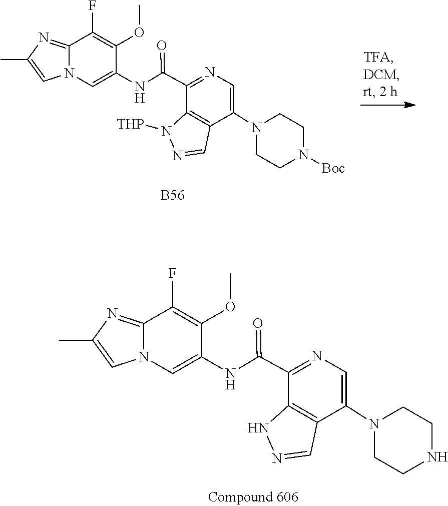

To a solution of tert-butyl 4-[7-[(8-fluoro-7-methoxy-2-methyl-imidazo[1,2-a]pyridin-6-yl)carbamoyl]-1-tetrahydropyran-2-yl-pyrazolo[3,4-c]pyridin-4-yl]piperazine-1-carboxylate (B56, 70 mg, 115 μmol) in DCM (2 mL) was added TFA (0.5 mL), and the reaction was stirred for 2 h at rt. The resulting mixture was concentrated under reduced pressure and purified by reversed-phase flash chromatography (Condition 3, Gradient 11) to afford N-(8-fluoro-7-methoxy-2-methyl-imidazo[1,2-a]pyridin-6-yl)-4-piperazin-1-yl-1H-pyrazolo[3,4-c]pyridine-7-carboxamide (Compound 606, 25 mg, 59 μmol, 51% yield) as a solid. LCMS (ES, m/z): 425 [M+H]⁺. ¹H NMR (400 MHz, DMSO-d₆) δ 13.56 (s, 1H), 10.03 (s, 1H), 9.23 (s, 1H), 8.48 (s, 1H), 7.89 (s, 1H), 7.81 (d, J=3.1 Hz, 1H), 4.16 (d, J=2.6 Hz, 3H), 3.62-3.55 (m, 4H), 2.93 (t, J=5.0 Hz, 4H), 2.33 (s, 3H).

### Example 85: Synthesis of Compound 361

### Synthesis of B57

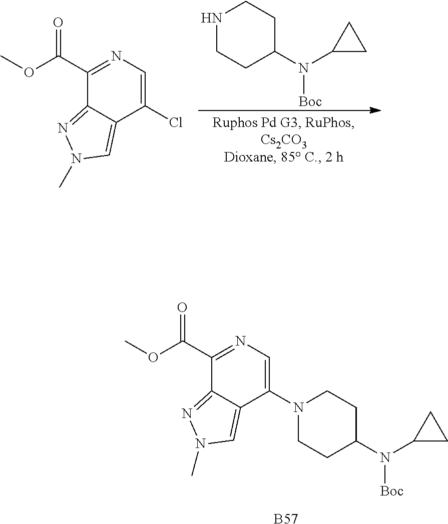

To a stirred mixture of methyl 4-chloro-2-methyl-pyrazolo[3,4-c]pyridine-7-carboxylate (0.58 g, 2.57 mmol) and tert-butyl N-cyclopropyl-N-(4-piperidyl)carbamate (741 mg, 3.08 mmol) in Dioxane (6 mL) were added Cs₂CO₃ (2.5 g, 7.7 mmol), Ruphos (240 mg, 514.1 μmol) and RuPhos Pd G3 (215 mg, 257.1 μmol), and the reaction was stirred for 2 h at 85° C. under N2. The mixture was allowed to cool down to room temperature, concentrated under reduced pressure, and purified by silica gel column chromatography (83% EtOAc in PE) to afford methyl 4-[4-[tert-butoxycarbonyl(cyclopropyl)amino]-1-piperidyl]-2-methyl-pyrazolo-[3,4-c]pyridine-7-carboxylate (B57, 700 mg, 1.63 mmol, 63% yield) as a solid. LCMS (ES, m/z): 430 [M+H]⁺.

### Synthesis of B58

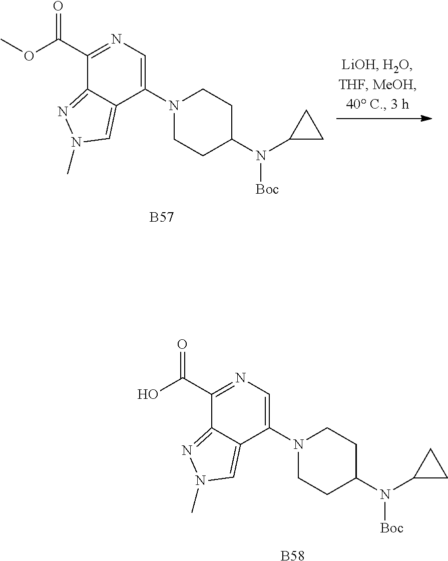

To a stirred mixture of methyl 4-[4-[tert-butoxycarbonyl(cyclopropyl)amino]-1-piperidyl]-2-methyl-pyrazolo[3,4-c]pyridine-7-carboxylate (B57, 0.7 g, 1.63 mmol) in THF (4 mL) and H₂O (2 mL) and MeOH (4 mL) was added LiOH·H₂O (205.2 mg, 4.9 mmol), and the reaction was stirred for 4 h at 40° C. The resulting mixture was neutralized to pH 5 with 1 N of HCl (aq.). The solid was collected by filtration and washed with water (4 mL) and MTBE (2 mL). The solid was dried to afford 4-[4-[tert-butoxycarbonyl(cyclopropyl)amino]-1-piperidyl]-2-methyl-pyrazolo[3,4-c]pyridine-7-carboxylic acid (B58, 0.5 g, 1.2 mmol, 74% yield) as a solid. LCMS (ES, m/z): 416 [M+H]⁺.

### Synthesis of B59

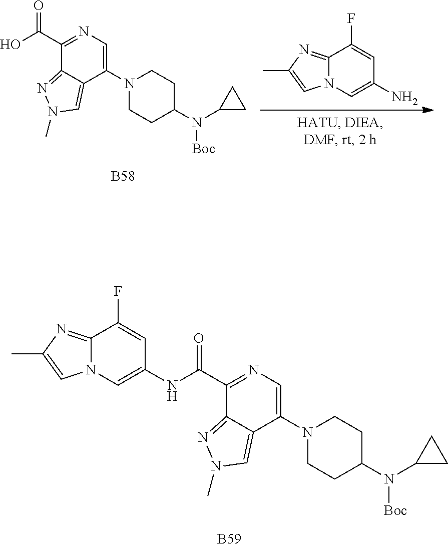

To a stirred mixture of 4-[4-[tert-butoxycarbonyl(cyclopropyl)amino]-1-piperidyl]-2-methyl-pyrazolo[3,4-c]pyridine-7-carboxylic acid (B58, 0.16 g, 385 μmol) and 8-fluoro-2-methylimidazo[1,2-a]pyridin-6-amine (70 mg, 423.6 μmol) in DMF (2 mL) was added DIEA (149 mg, 1.16 mmol, 201.2 μL) and HATU (220 mg, 577.6 μmol), and the reaction was stirred for 2 h at room temperature. The resulting mixture was diluted with water (10 mL). The solid was collected by filtration and washed with water (4 mL). The solid was dried to afford tert-butyl-N-cyclopropyl-N-[1-[7-[(8-fluoro-2-methyl-imidazo[1,2-a]pyridin-6-yl)carbamoyl]-2-methyl-pyrazolo[3,4-c]pyridin-4-yl]-4-piperidyl]carbamate (B59, 0.14 g, 248.8 μmol, 65% yield) as a solid. LCMS (ES, m/z): 563 [M+H]⁺.

### Synthesis of Compound 361

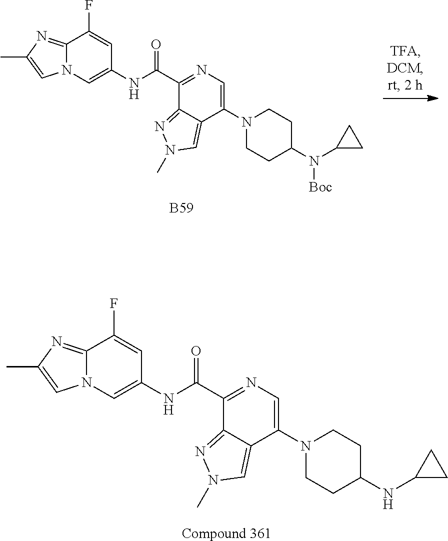

To a solution of tert-butyl N-cyclopropyl-N-[1-[7-[(8-fluoro-2-methyl-imidazo[1,2-a]-pyridin-6-yl)carbamoyl]-2-methyl-pyrazolo[3,4-c]pyridin-4-yl]-4-piperidyl]carbamate (B59, 0.12 g, 213.3 μmol) in DCM (2 mL) was added TFA (24.3 mg, 213.3 μmol), and the reaction was stirred for 2 h at room temperature. The resulting mixture was concentrated under reduced pressure and adjusted to pH 8 with saturated NaHCO₃ (aq.). The residue was purified by reversed-phase flash chromatography (Condition 3, Gradient 10) to 4-[4-(cyclopropylamino)-1-piperidyl]-N-(8-fluoro-2-methyl-imidazo[1,2-a]pyridin-6-yl)-2-methyl-pyrazolo[3,4-c]pyridine-7-carboxamide (Compound 361, 0.024 g, 51.2 μmol, 24% yield) as a solid. LCMS (ES, m/z): 463 [M+H]⁺. ¹H NMR (400 MHz, DMSO-d₆) δ 10.59 (s, 1H), 9.27 (d, J=1.6 Hz, 1H), 8.86 (s, 1H), 7.89 (d, J=3.1 Hz, 1H), 7.65 (s, 1H), 7.53 (dd, J=12.9, 1.6 Hz, 1H), 4.27 (s, 3H), 3.95 (d, J=13.0 Hz, 2H), 3.15 (t, J=11.4 Hz, 2H), 2.81 (s, 1H), 2.35 (s, 3H), 2.28 (s, 1H), 2.12 (dq, J=6.7, 3.4 Hz, 1H), 2.02 (d, J=12.4 Hz, 2H), 1.48 (q, J=9.8 Hz, 2H), 0.45-0.36 (m, 2H), 0.28-0.20 (m, 2H).

An analogous method was followed to obtain the following compounds.

| | Starting | |
|---|---|---|
| Compound | Material | Characterization |
| 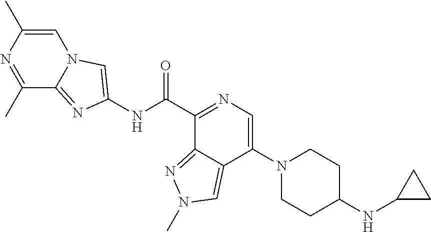 [SMILES: Cc1cn2cc(NC(=O)c3ncc(N4CCC(NC5CC5)CC4)c4cn(C)nc34)nc2c(C)n1] Compound 264 | 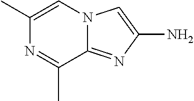 | The residue was purified by reversed- phase flash chromatography (Condition 3, Gradient 8) to afford 4-[4- (cyclopropylamino)-1-piperidyl]-N- (6,8-dimethylimidazo[1,2-a]pyrazin-2- yl)-2-methyl-pyrazolo[3,4-c]pyridine- 7-carboxamide (Compound 264, 10 mg, 21.1 μmol, 12% yield) as a solid. LCMS (ES, m/z): 460 [M + H]⁺ 1H NMR (400 MHZ, DMSO-d₆) δ 11.02 (s, 1H), 8.95 (s, 1H), 8.34 (d, J = 3.2 Hz, 2H), 7.75 (s, 1H), 4.32 (s, 3H), 4.00 (d, J = 13.0 Hz, 2H), 3.20 (t, J = 11.4 Hz, 2H), 2.85-2.75 (m, 1H), 2.69 (s, 3H), 2.38 (s, 3H), 2.28 (s, 1H), 2.13 (tt, J = 6.7, 3.6 Hz, 1H), 2.02 (d, J = 12.2 Hz, |
| | | 2H), 1.47 (q, J = 10.2 Hz, 2H), 0.45- |
| | | 0.36 (m, 2H), 0.27-0.20 (m, 2H). |

### Example 86: Synthesis of Compound 209

### Synthesis of B60

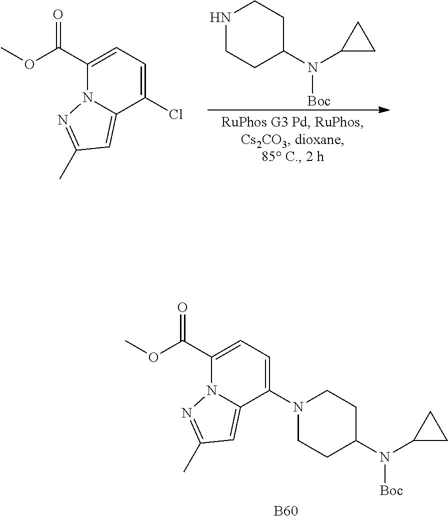

To a solution of methyl 4-chloro-2-methyl-pyrazolo[1,5-a]pyridine-7-carboxylate (400 mg, 1.78 mmol) and tert-butyl N-cyclopropyl-N-(4-piperidyl)carbamate (514 mg, 2.14 mmol) in dioxane (8 mL) were added Cs₂CO₃ (1.16 g, 3.56 mmol) and RuPhos (166 mg, 356.1 μmol) and RuPhos Pd G3 (149 mg, 178.1 μmol), and the reaction was stirred for 3 h at 80° C. under N2. The resulting mixture was concentrated under reduced pressure and purified by silica gel column chromatography (67% EtOAc in PE) to afford methyl 4-[4-[tert-butoxycarbonyl(cyclopropyl)amino]-1-piperidyl]-2-methyl-pyrazolo[1,5-a]pyridine-7-carboxylate (B60, 490 mg, 1.14 mmol, 64% yield) as a solid. LCMS (ES, m/z): 429 [M+H]⁺.

### Synthesis of B61

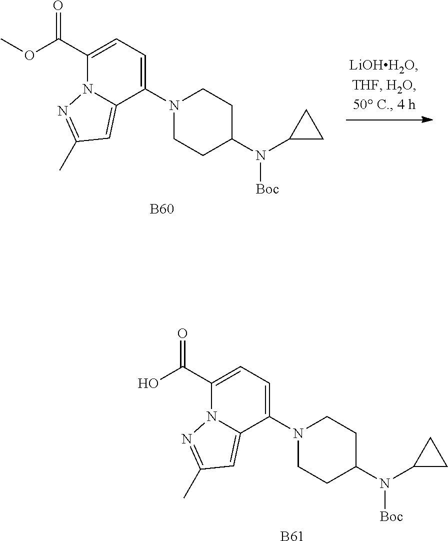

To a stirred solution of methyl 4-[4-[tert-butoxycarbonyl(cyclopropyl)amino]-1-piperidyl]-2-methyl-pyrazolo[1,5-a]pyridine-7-carboxylate (480 mg, 1.12 mmol) in THF (5 mL), H₂O (5 mL) was added LiOH (188 mg, 7.84 mmol) in portions, and the rection was stirred for 4 h at 50° C. The resulting mixture was diluted with water (20 mL), acidified to pH 6 with HCl (2N), and extracted with EtOAc (2×20 mL). The combined organic layers were washed with brine (20 mL), dried over anhydrous Na₂SO₄, and filtered. The filtrate was concentrated under reduced pressure to afford 4-[4-[tert-butoxycarbonyl(cyclopropyl)amino]-1-piperidyl]-2-methyl-pyrazolo[1,5-a]pyridine-7-carboxylic acid (B61, 390 mg, 940.9 μmol, 84% yield) as a solid. LCMS (ES, m/z): 415 [M+H]⁺.

### Synthesis of B62

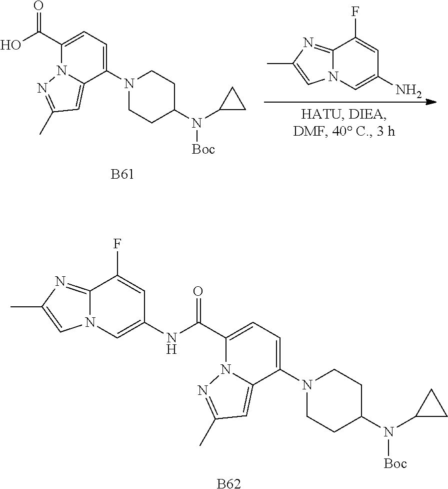

To a stirred mixture of 4-[4-[tert-butoxycarbonyl(cyclopropyl)amino]-1-piperidyl]-2-methyl-pyrazolo[1,5-a]pyridine-7-carboxylic acid (380 mg, 916.8 μmol), 8-fluoro-2-methyl-imidazo[1,2-a]pyridin-6-amine (166 mg, 1.01 mmol) and DIEA (592 mg, 4.58 mmol) in DMF (8 mL) was added HATU (523 mg, 1.38 mmol), and the reaction was stirred for 3 h at room temperature. The resulting mixture was quenched with water (30 mL) and extracted with EtOAc (2×40 mL). The combined organic layers were washed with brine (40 mL), dried over anhydrous Na₂SO₄, and filtered. The filtrate was concentrated under reduced pressure and purified by silica gel column chromatography (67% EtOAc in PE) to afford tert-butyl N-cyclopropyl-N-[1-[7-[(8-fluoro-2-methyl-imidazo[1,2-a]pyridin-6-yl)carbamoyl]-2-methyl-pyrazolo[1,5-a]pyridin-4-yl]-4-piperidyl]carbamate (B62, 300 mg, 534.1 μmol, 58% yield) as a solid. LCMS (ES, m/z): 562 [M+H]⁺.

### Synthesis of Compound 209

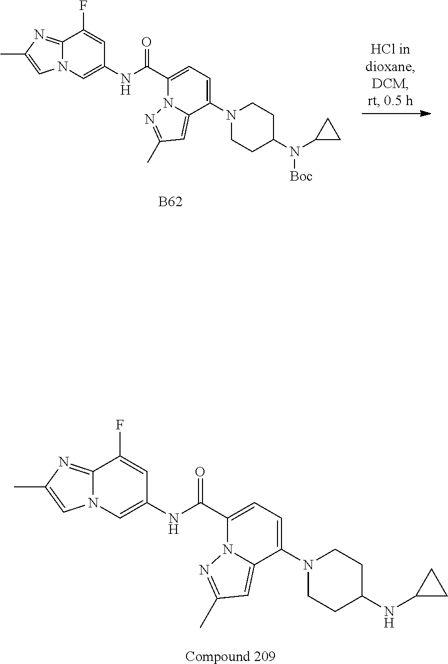

To a solution of tert-butyl N-cyclopropyl-N-[1-[7-[(8-fluoro-2-methyl-imidazo[1,2-a]-pyridin-6-yl)carbamoyl]-2-methyl-pyrazolo[1,5-a]pyridin-4-yl]-4-piperidyl]carbamate (390 mg, 694.4 μmol) in DCM (4 mL) was added HCl (4.0 M in 1,4-dioxane, 1.5 mL), and the reaction was stirred for 0.5 h at room temperature. The resulting mixture was concentrated under reduced pressure and adjusted to pH 8 with saturated NaHCO₃ (aq.). The resulting mixture was diluted with water (5 mL) and extracted with DCM/MeOH (20/1, 2×10 mL). The combined organic layers were washed with water (20 mL), brine (20 mL) and dried over anhydrous Na₂SO₄, and filtered. The filtrate was concentrated under reduced pressure and purified by trituration with Et₂O (10 mL), to afford 4-[4-(cyclopropylamino)-1-piperidyl]-N-(8-fluoro-2-methyl-imidazo[1,2-a]pyridin-6-yl)-2-methyl-pyrazolo[1,5-a]pyridine-7-carboxamide (Compound 209, 190 mg, 411.7 μmol, 59% yield) as a solid. LCMS (ES, m/z): 462 [M+H]⁺. ¹H NMR (400 MHz, DMSO-d₆) δ 12.71 (s, 1H), 9.20 (d, J=1.6 Hz, 1H), 7.92 (d, J=3.1 Hz, 1H), 7.73 (d, J=8.1 Hz, 1H), 7.38 (dd, J=12.2, 1.7 Hz, 1H), 6.72 (d, J=8.2 Hz, 1H), 6.67 (s, 1H), 3.70 (d, J=12.5 Hz, 2H), 2.93 (t, J=11.5 Hz, 2H), 2.75 (dt, J=10.0, 5.7 Hz, 1H), 2.57 (s, 3H), 2.36 (d, J=0.9 Hz, 3H), 2.26 (s, 1H), 2.12 (tt, J=6.7, 3.6 Hz, 1H), 2.05-1.96 (m, 2H), 1.50 (q, J=9.8 Hz, 2H), 0.40 (td, J=6.3, 4.1 Hz, 2H), 0.27-0.20 (m, 2H).

An analogous method was followed to obtain the following compound.

| | Starting | |
|---|---|---|
| Compound | Material | Characterization |
| 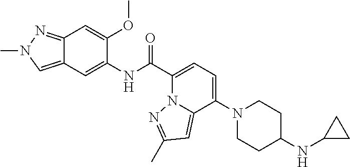 [SMILES: COc1cc2nn(C)cc2cc1NC(=O)c1ccc(N2CCC(NC3CC3)CC2)c2cc(C)nn12] Compound 619 | 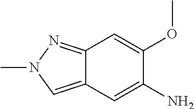 | Modification: Step 1 was ran at 40° C. for 3 h. Step 2 was ran using TFA and DCM for 1 h at room temperature. The residue was purified by reversed- phase flash chromatography (Condition 3, Gradient 4) to afford 4-[4- (cyclopropylamino)-1-piperidyl]-N-(6- methoxy-2-methyl-indazol-5-y1)-2- methyl-pyrazolo[1,5-a]-pyridine-7- carboxamide (Compound 619, 27 mg, 57 μmol, 55% yield) as a solid. LCMS (ES, m/z): 474 [M + H]⁺. ¹H NMR (400 MHz, DMSO-d₆) δ 13.30 (s, 1H), 8.85 (s, 1H), 8.23 (s, 1H), 7.80 (d, J = 8.1 Hz, 1H), 7.11 (s, 1H), 6.75 (d, J = 8.2 Hz, 1H), 6.68 (s, 1H), 4.09 (s, 3H), 4.04 |
| | | (s, 3H), 3.72 (d, J = 12.4 Hz, 2H), 2.93 |
| | | (t, J = 11.5 Hz, 2H), 2.80 (s, 1H), 2.59 |
| | | (s, 3H), 2.17 (s, 1H), 2.03 (d, J = 12.3 |
| | | Hz, 2H), 1.53 (d, J = 11.5 Hz, 2H), 0.43 |
| | | (d, J = 6.2 Hz, 2H), 0.28 (s, 2H). |
| 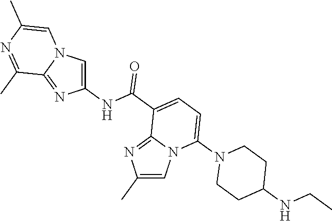 [SMILES: CCNC1CCN(c2ccc(C(=O)Nc3cn4cc(C)nc(C)c4n3)c3nc(C)cn23)CC1] Compound 612 | 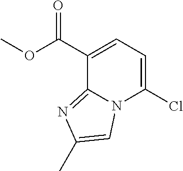 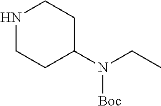 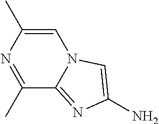 | Modification: Step 1 was ran at 80° C. for 16 h. Step 2 was ran at room temperature for 16 h. Step 3 was ran at room temperature for 16 h. The crude product was purified by Prep-HPLC (Condition 5, Gradient 22) to afford N-(6,8-dimethyl-imidazo- [1,2-a]pyrazin-2-y1)-5-[4- (ethylamino)-1-piperidyl]-2-methyl- imidazo[1,2-a]pyridine-8-carboxamide (Compound 612, 18.3 mg, 41 μmol, 28% yield) as a solid. LCMS (ES, m/z): 447 [M + H]⁺ ¹H NMR (400 MHZ, DMSO-d₆) δ 12.74 (s, 1H), 8.34 (d, J = 6.9 Hz, 2H), 8.15 (d, J = 7.9 Hz, 1H), 7.62 (s, 1H), 6.63 (d, J = 7.9 Hz, 1H), 3.52 (d, J = 11.6 Hz, 2H), 2.91 (t, J = 11.2 Hz, 2H), 2.71-2.60 (m, 4H), 2.62 (q, J = 7.0 Hz, 2H), 2.53 (s, 3H), 2.38 (s, 3H), 2.01 (d, J = 12.4 Hz, 2H), 1.56 (q, J = 10.9 Hz, 2H), 1.06 (t, J = 7.1 Hz, 3H). |
| 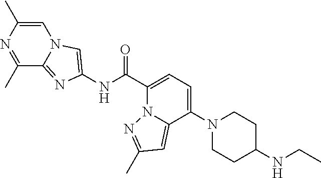 [SMILES: CCNC1CCN(c2ccc(C(=O)Nc3cn4cc(C)nc(C)c4n3)n3nc(C)cc23)CC1] Compound 615 | 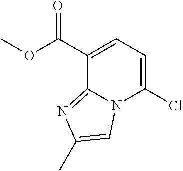 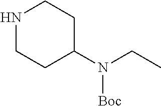 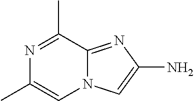 | Modification: Step 3 was ran for 3 h at room temperature. Step 4 was ran using TFA in DCM at room temperature for 1 h. The residue was purified by reversed- phase flash chromatography (Condition 3, Gradient 11) to afford N-(6,8- dimethylimidazo[1,2-a]-pyrazin-2-y1)- 4-[4-(ethylamino)-1-piperidyl]-2- methyl-pyrazolo[1,5-a]-pyridine-7- carboxamide (Compound 615, 28 mg, 62.7 μmol, 43% yield) as a solid. LCMS (ES, m/z): 447 [M + H]⁺ ¹H NMR (400 MHZ, DMSO-d₆) δ 13.11 (s, 1H), 8.35 (d, J = 8.8 Hz, 2H), 7.82 (d, J = 8.1 Hz, 1H), 6.78-6.69 (m, 2H), 3.73 (d, J = 12.5 Hz, 2H), 2.95 (t, J = 11.6 Hz, 2H), 2.71 (s, 3H), 2.62 (q, J = 7.0 Hz, 2H), 2.58 (s, 3H), 2.52 (s, 1H), 2.38 (s, 3H), 1.97 (d, J = 12.5 Hz, 2H), 1.54-1.44 (m, 2H), 1.05 (t, J = 7.1 Hz, 3H). |
| 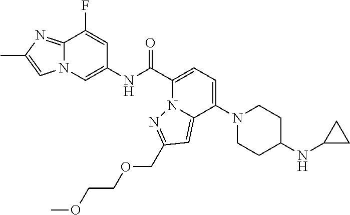 [SMILES: COCCOCc1cc2c(N3CCC(NC4CC4)CC3)ccc(C(=O)Nc3cc(F)c4nc(C)cn4c3)n2n1] Compound 629 | 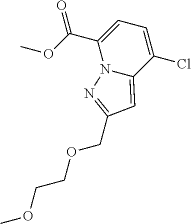 C223 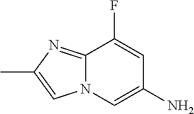 | Modification: Step 1 was ran at 70° C. for 3 h. Step 2 was ran at room temperature for 4 h. Step 3 was ran for 3 h at room temperature. Step 4 was ran for 2 h at room temperature. The residue was purified by reversed- phase flash chromatography (Condition 3, Gradient 18) to afford 4-[4- (cyclopropylamino)-1-piperidyl]-N-(8- fluoro-2-methyl-imidazo[1,2-a]- pyridin-6-y1)-2-(2-methoxyethoxy- methyl)-pyrazolo[1,5-a]pyridine-7- carboxamide (Compound 629, 0.005 g, 9.34 μmol, 16% yield) as a solid. LCMS (ES, m/z): 536 [M + H]⁺ ¹H NMR (300 MHZ, Methanol-d4) δ 9.01- 8.90 (m, 1H), 7.78-7.65 (m, 1H), 7.56 (d, J = 3.6 Hz, 1H), 7.14-6.98 (m, 1H), 6.67 (d, J = 10.3 Hz, 1H), 6.56 (q, J = 8.2 Hz, 1H), 4.83 (d, J = 4.4 Hz, 2H), 3.84-3.69 (m, 4H), 3.65 (dd, J = 5.8, 3.3 Hz, 2H), 3.40 (s, 3H), 2.83 (s, 3H), 2.37 (d, J = 3.0 Hz, 3H), 2.22 (tt, J = 7.0, 3.8 Hz, 1H), 2.12 (d, J = 12.7 Hz, 2H), 1.59 (d, J = 12.1 Hz, 2H), 1.29 (s, 1H), 0.59- |
| | | 0.49 (m, 2H), 0.41 (q, J = 3.5, 2.9 Hz, |
| | | 2H). |

### Example 87: Synthesis of Compound 607

### Synthesis of B63

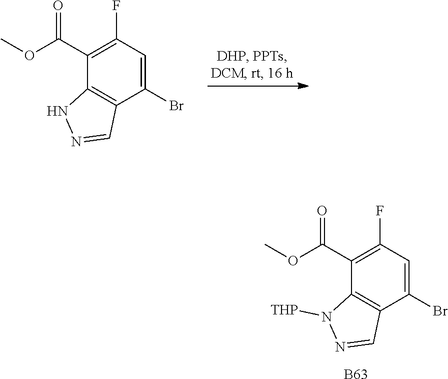

A mixture of methyl 4-bromo-6-fluoro-1H-indazole-7-carboxylate (500 mg, 1.83 mmol), DHP (1.65 g, 45.78 mmol) and PPTs (920 mg, 3.66 mmol) in DCM (10 mL) was stirred for 16 h at room temperature. The resulting mixture was diluted with water (20 mL) and extracted with DCM (3×20 mL). The combined organic layers were washed with brine (10 mL), dried over anhydrous Na₂SO₄, and filtered. The filtrate was concentrated under reduced pressure and purified by silica gel column chromatography (20% EtOAc in PE) to afford methyl 4-bromo-6-fluoro-1-tetrahydropyran-2-yl-indazole-7-carboxylate (B63, 420 mg, 1.18 mmol, 64% yield) as a solid. LCMS (ES, m/z): 357 [M+H]⁺.

### Synthesis of B64

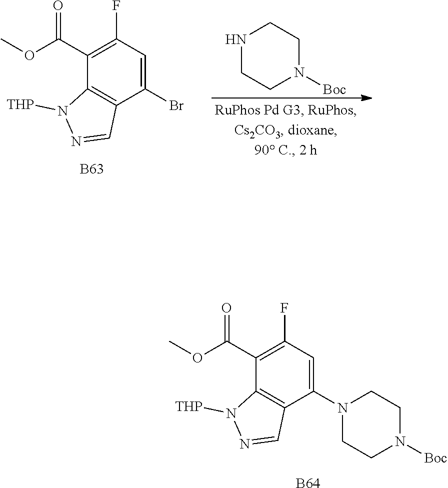

To a stirred mixture of 4-methyl 4-bromo-6-fluoro-1-tetrahydropyran-2-yl-indazole-7-carboxylate (430 mg, 1.20 mmol) and tert-butyl piperazine-1-carboxylate (269 mg, 1.44 mmol) in Dioxane (8 mL) was added RuPhos Pd G3 (101 mg, 120.4 μmol), Cs₂CO₃ (785 mg, 2.41 mmol) and Ruphos (112 mg, 240.8 μmol) in portions, and the reaction was stirred for 2 h at 90° C. under N₂. The resulting mixture was allowed to cool down to room temperature, concentrated under reduced pressure, and purified by silica gel column chromatography (50% EtOAc in PE) to afford methyl 4-(4-tert-butoxycarbonylpiperazin-1-yl)-6-fluoro-1-tetrahydropyran-2-yl-indazole-7-carboxylate (B64, 420 mg, 908.1 μmol, 75% yield) as a solid. LCMS (ES, m/z): 463 [M+H]⁺.

### Synthesis of B65

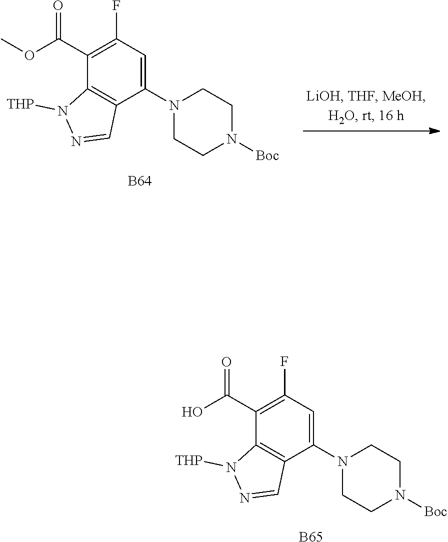

To a stirred mixture of methyl 4-(4-tert-butoxycarbonylpiperazin-1-yl)-6-fluoro-1-tetrahydropyran-2-yl-indazole-7-carboxylate (380 mg, 821.6 μmol) in THF (5 mL), H₂O (5 mL) and MeOH (1 mL) was added LiOH (99 mg, 4.11 mmol), and the reaction was stirred for 16 h at room temperature. The resulting mixture was acidified to pH 5 with HCl (1 M) and extracted with DCM (3×15 mL). The combined organic layers were washed with brine (10 mL), dried over anhydrous Na₂SO₄, and filtered. The filtrate was concentrated under reduced pressure, to afford 4-(4-tert-butoxycarbonylpiperazin-1-yl)-6-fluoro-1-tetrahydropyran-2-yl-indazole-7-carboxylic acid (320 mg, 713.5 μmol, 87% yield) as a solid. LCMS (ES, m/z): 449 [M+H]⁺.

### Synthesis of B66

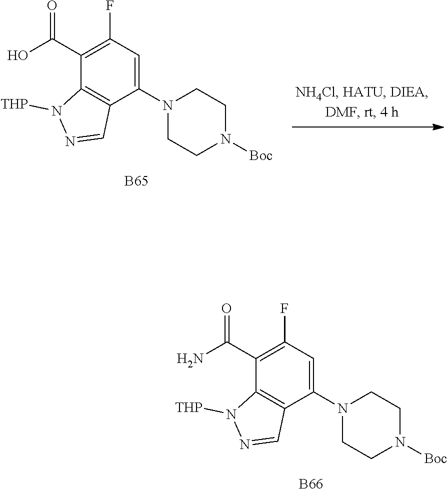

A mixture of 4-(4-tert-butoxycarbonylpiperazin-1-yl)-6-fluoro-1-tetrahydropyran-2-yl-indazole-7-carboxylic acid (150 mg, 334.5 μmol), HATU (165 mg, 434.8 μmol) and DIEA (130 mg, 1.0 mmol) in DMF (3 mL) was stirred for 5 min at room temperature. To the above mixture was added NH₄Cl (27 mg, 501.7 μmol), and the reaction was stirred for an additional 4 h at room temperature. The resulting mixture was precipitated by the addition of water (10 mL). The precipitated solids were collected by filtration and washed with water (3×5 mL) to afford tert-butyl 4-(7-carbamoyl-6-fluoro-1-tetrahydropyran-2-yl-indazol-4-yl)piperazine-1-carboxylate (B66, 100 mg, 223.5 μmol, 67% yield) as a solid. LCMS (ES, m/z): 364 [M+H]⁺.

### Synthesis of B67

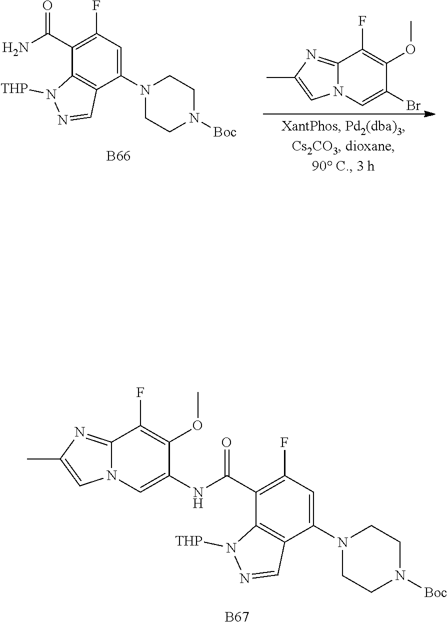

To a stirred mixture of tert-butyl 4-(7-carbamoyl-6-fluoro-1H-indazol-4-yl)piperazine-1-carboxylate (150 mg, 412.8 μmol) and 6-bromo-8-fluoro-7-methoxy-2-methyl-imidazo[1,2-a]-pyridine (128 mg, 495.3 μmol) in Dioxane (3 mL) was added Xantphos (48 mg, 82.6 μmol), Cs₂CO₃ (269 mg, 825.6 μmol) and Pd₂(dba)₃ (38 mg, 41.28 μmol), and the reaction was stirred for 3 h at 90° C. under N₂. The resulting mixture was cooled to room temperature, concentrated under reduced pressure, and purified by silica gel column chromatography (100% EtOAc) to afford tert-butyl 4-[6-fluoro-7-[(8-fluoro-7-methoxy-2-methyl-imidazo[1,2-a]pyridin-6-yl)carbamoyl]-1-tetrahydropyran-2-yl-indazol-4-yl]-piperazine-1-carboxylate (B67, 100 mg, 159.8 μmol, 39% yield) as a solid. LCMS (ES, m/z): 542 [M+H]⁺.

### Synthesis of Compound 607

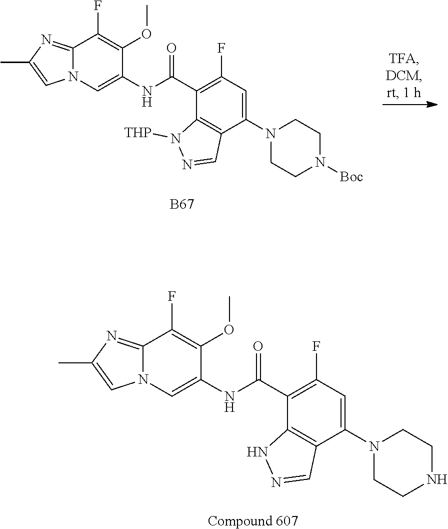

To a stirred mixture of tert-butyl 4-[6-fluoro-7-[(8-fluoro-7-methoxy-2-methyl-imidazo[1,2-a]pyridin-6-yl)carbamoyl]-1H-indazol-4-yl]piperazine-1-carboxylate (80 mg, 147.7 μmol) in DCM (2 mL) were added TFA (1 mL), and the reaction was stirred for 1 h at room temperature. The resulting mixture was concentrated under reduced pressure and purified by reversed-phase flash chromatography (Condition 5, Gradient 1) to afford 6-fluoro-N-(8-fluoro-7-methoxy-2-methyl-imidazo[1,2-a]pyridin-6-yl)-4-piperazin-1-yl-1H-indazole-7-carboxamide (Compound 607, 13 mg, 29.5 μmol, 20% yield) as a solid. LCMS (ES, m/z): 442 [M+H]⁺. ¹H NMR (400 MHz, DMSO-d₆) δ 13.21 (s, 1H), 9.18 (s, 2H), 8.34 (s, 1H), 7.78 (d, J=3.0 Hz, 1H), 6.43 (d, J=16.6 Hz, 1H), 4.11 (d, J=2.2 Hz, 3H), 3.48 (t, J=4.9 Hz, 4H), 2.89 (t, J=4.9 Hz, 4H), 2.51 (s, 8H), 2.33 (s, 3H).

### Example 88: Synthesis of Compound 285

### Synthesis of B68

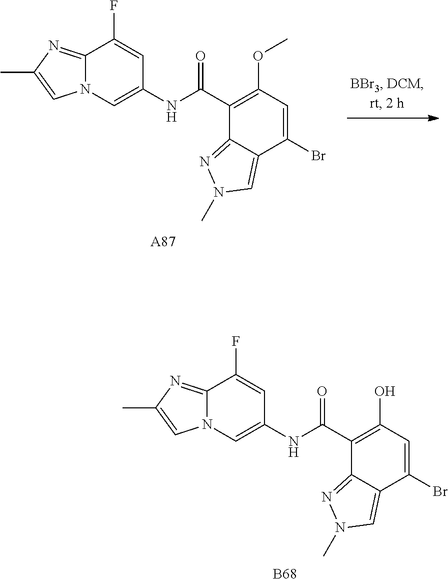 [SMILES: COc1cc(Br)c2cn(C)nc2c1C(=O)Nc1cc(F)c2nc(C)cn2c1 ; "A87" ; "B68" ; Cc1cn2cc(NC(=O)c3c(O)cc(Br)c4cn(C)nc34)cc(F)c2n1]

To a stirred solution of 4-bromo-N-(8-fluoro-2-methyl-imidazo[1,2-a]pyridin-6-yl)-6-methoxy-2-methyl-indazole-7-carboxamide (A87, 500 mg, 1.16 mmol) in DCE (5 mL) was added tribromoborane (0.5 mL) in portions at 0° C. The resulting mixture was stirred for 2 h at 50° C. The reaction was quenched with MeOH at 0° C., concentrated under reduced pressure and purified by reverse flash chromatography (Condition 1, Gradient 1) to afford 4-bromo-N-(8-fluoro-2-methyl-imidazo[1,2-a]pyridin-6-yl)-6-hydroxy-2-methyl-indazole-7-carboxamide (B68, 300 mg, 717.3 μmol, 62% yield) as a solid. LCMS (ES, m/z): 418 [M+H]⁺.

### Synthesis of B69

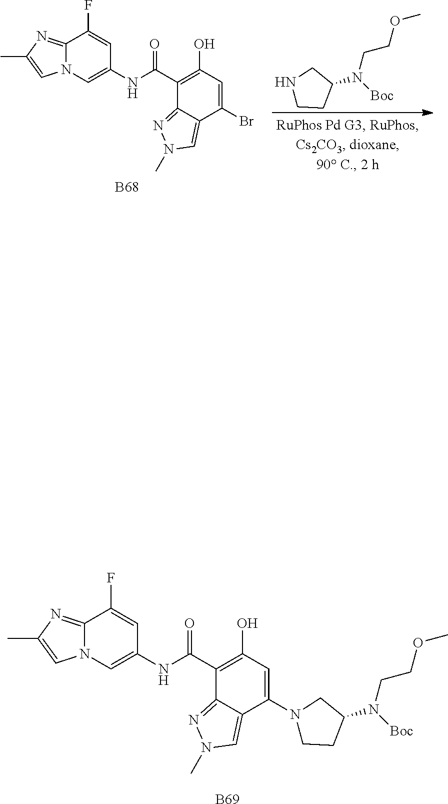

To a stirred mixture of 4-bromo-N-(8-fluoro-2-methyl-imidazo[1,2-a]pyridin-6-yl)-6-hydroxy-2-methyl-indazole-7-carboxamide (B68, 300 mg, 717.3 μmol) and tert-butyl (R)-(2-methoxyethyl)(pyrrolidin-3-yl)carbamate (210.3 mg, 860.8 μmol) in dioxane (5 mL) were added RuPhos Pd G3 (59.99 mg, 71.73 μmol), Ruphos (66.95 mg, 143.5 μmol) and Cs₂CO₃ (701.2 mg, 2.15 mmol) at room temperature under N₂. The resulting mixture was stirred for 2 h at 90° C. under N2. The mixture was allowed to cool down to room temperature. The resulting mixture was concentrated under reduced pressure and purified by silica gel column chromatography (50% EtOAc in PE) to afford tert-butyl N-[(3R)-1-[7-[(8-fluoro-2-methyl-imidazo[1,2-a]pyridin-6-yl)carbamoyl]-6-hydroxy-2-methyl-indazol-4-yl]pyrrolidin-3-yl]-N-(2-methoxyethyl)-carbamate (B69, 150 mg, 257.9 μmol, 36% yield) as a solid. LCMS (ES, m/z): 582 [M+H]⁺.

### Synthesis of Compound 285

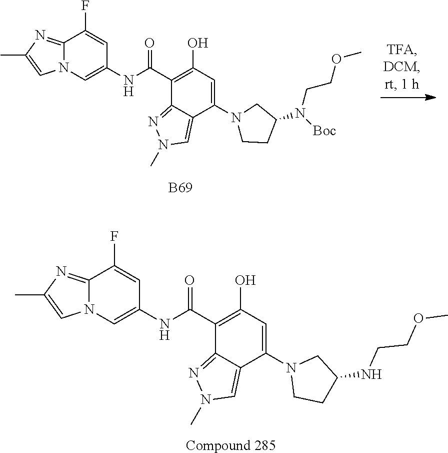

To a solution of tert-butyl N-[(3R)-1-[7-[(8-fluoro-2-methyl-imidazo[1,2-a]pyridin-6-yl)carbamoyl]-6-hydroxy-2-methyl-indazol-4-yl]pyrrolidin-3-yl]-N-(2-methoxyethyl)carbamate (B69, 150 mg, 257.9 μmol) in DCM (2 mL) was added TFA (298.0 mg, 2.61 mmol, 0.2 mL). The reaction was stirred for 1 h at rt. The resulting mixture was concentrated under reduced pressure and adjusted to pH 8 with saturated NaHCO₃ (aq.). The resulting mixture was diluted with water (10 mL) and extracted with DCM/MeOH (20/1) (3×10 mL). The combined organic layers were washed with brine (10 mL) and dried over anhydrous Na₂SO₄, and filtered. The filtrate was concentrated under reduced pressure and purified by reversed-phase flash chromatography (Condition 3, Gradient 6) to afford N-(8-fluoro-2-methyl-imidazo[1,2-a]pyridin-6-yl)-6-hydroxy-4-[(3R)-3-(2-methoxyethylamino)pyrrolidin-1-yl]-2-methyl-indazole-7-carboxamide (Compound 285, 50.4 mg, 104.7 μmol, 41% yield) as a solid. LCMS (ES, m/z): 482 [M+H]⁺. ¹H NMR (400 MHz, DMSO-d₆) δ 13.49 (s, 1H), 11.01 (s, 1H), 9.03 (s, 1H), 8.67 (s, 1H), 7.89 (d, J=3.0 Hz, 1H), 7.31 (d, J=12.2 Hz, 1H), 5.49 (s, 1H), 4.17 (s, 3H), 3.75 (s, 1H), 3.68 (s, 1H), 3.59 (s, 1H), 3.49-3.37 (m, 4H), 3.26 (s, 3H), 2.76 (t, J=5.7 Hz, 2H), 2.35 (s, 3H), 2.20-2.11 (m, 1H), 1.90 (dd, J=12.2, 6.4 Hz, 2H).

### Example 89: Synthesis of Compound 291

### Synthesis of B70

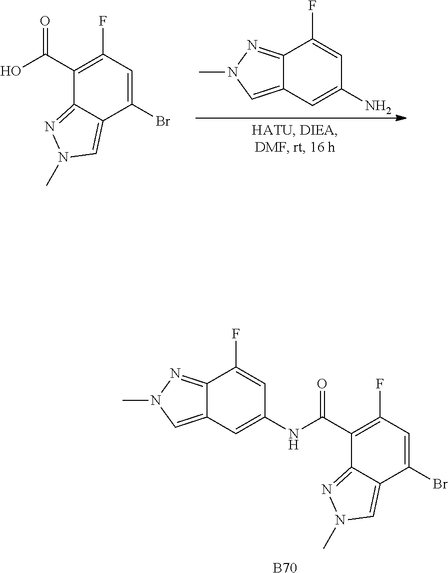

A mixture of 4-bromo-6-fluoro-2-methyl-indazole-7-carboxylic acid (600 mg, 2.20 mmol), HATU (835 mg, 2.20 mmol) and DIEA (284 mg, 2.20 mmol) in DMF (4 mL) was stirred for 5 min at room temperature. To the above mixture was added 7-fluoro-2-methyl-indazol-5-amine (544 mg, 3.30 mmol), and the reaction was stirred for an additional 16 h at room temperature. The product was precipitated by the addition of water (10 mL). The precipitated solids were collected by filtration and washed with water (2×10 mL) to afford 4-bromo-6-fluoro-N-(7-fluoro-2-methyl-indazol-5-yl)-2-methyl-indazole-7-carboxamide (B70, 800 mg, 1.90 mmol, 87% yield) as a solid. LCMS (ES, m/z): 420 [M+H]⁺.

### Synthesis of B71

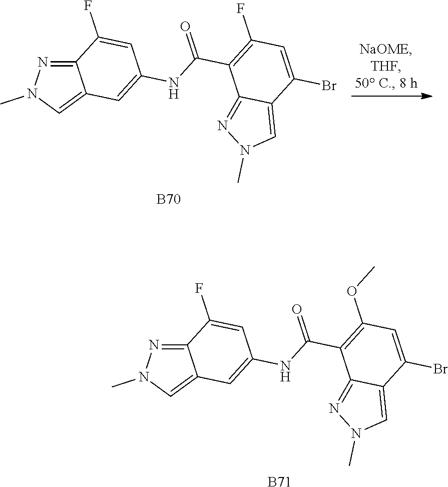 [SMILES: "B70" ; Cn1cc2cc(NC(=O)c3c(F)cc(Br)c4cn(C)nc34)cc(F)c2n1 ; "B71" ; COc1cc(Br)c2cn(C)nc2c1C(=O)Nc1cc(F)c2nn(C)cc2c1]

To a stirred solution of 4-bromo-6-fluoro-N-(7-fluoro-2-methyl-indazol-5-yl)-2-methyl-indazole-7-carboxamide (800 mg, 1.90 mmol) in THF (8 mL) were added NaOMe (309 mg, 5.71 mmol), and the reaction was stirred for 8 h at 50° C. The resulting mixture was concentrated under reduced pressure, diluted with water (10 mL), and extracted with EtOAc (3×15 mL). The combined organic layers were washed with brine (10 mL), dried over anhydrous Na₂SO₄, and filtered. The filtrate was concentrated under reduced pressure and purified by silica gel column chromatography (20% EtOAc in PE) to afford 4-bromo-N-(7-fluoro-2-methyl-indazol-5-yl)-6-methoxy-2-methyl-indazole-7-carboxamide (B71, 750 mg, 1.74 mmol, 91% yield) as a solid. LCMS (ES, m/z): 432 [M+H]⁺.

### Synthesis of B72

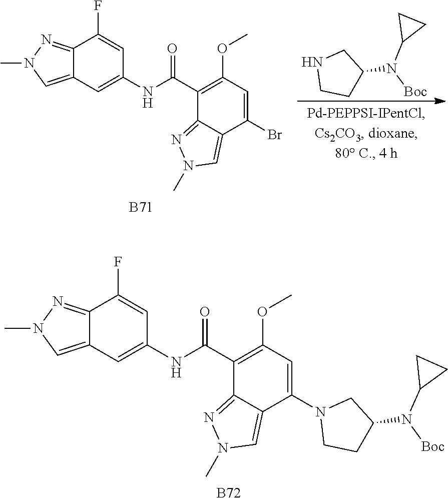

To a stirred mixture of 4-bromo-N-(7-fluoro-2-methyl-indazol-5-yl)-6-methoxy-2-methyl-indazole-7-carboxamide (180 mg, 417 μmol) and tert-butyl N-cyclopropyl-N-[(3R)-pyrrolidin-3-yl]carbamate (114 mg, 499.7 μmol) in Dioxane (3 mL) was added Pd-PEPPSI-IPentCl (29 mg, 41.64 μmol) and Cs₂CO₃ (271.4 mg, 832.9 μmol) in portions at room temperature under N₂. The resulting mixture was stirred for 4 h at 80° C. under N₂. The mixture was allowed to cool down to room temperature, concentrated under reduced pressure, and purified by silica gel column chromatography (33% EtOAc in PE) to afford tert-butyl N-cyclopropyl-N-[(3R)-1-[7-[(7-fluoro-2-methyl-indazol-5-yl)carbamoyl]-6-methoxy-2-methyl-indazol-4-yl]pyrrolidin-3-yl]carbamate (160 mg, 277 μmol, 67% yield) as a solid. LCMS (ES, m/z): 578 [M+H]⁺.

### Synthesis of Compound 291

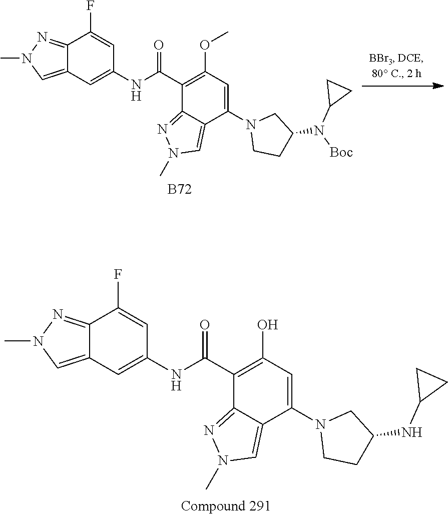

To a stirred solution of tert-butyl N-cyclopropyl-N-[(3R)-1-[7-[(7-fluoro-2-methyl-indazol-5-yl)carbamoyl]-6-methoxy-2-methyl-indazol-4-yl]pyrrolidin-3-yl]carbamate (130 mg, 225.1 μmol) in DCE (2 mL) was added tribromoborane (170 mg, 675.2 μmol), and the reaction was stirred for 2 h at 80° C. The resulting mixture was allowed to cool down to room temperature, concentrated under reduced pressure, and diluted with MeOH (3 mL). The resulting mixture was purified by reverse flash chromatography (Condition 5, Gradient 1) to afford 4-[(3R)-3-(cyclopropylamino)pyrrolidin-1-yl]-N-(7-fluoro-2-methyl-indazol-5-yl)-6-hydroxy-2-methyl-indazole-7-carboxamide (Compound 291, 30.4 mg, 65.6 μmol, 29% yield) as a solid. LCMS (ES, m/z): 464 [M+H]⁺. ¹H NMR (400 MHz, DMSO-d₆) δ 13.77 (s, 1H), 11.14 (s, 1H), 8.65 (s, 1H), 8.42 (d, J=2.7 Hz, 1H), 7.92 (s, 1H), 7.36 (d, J=13.1 Hz, 1H), 5.47 (s, 1H), 4.18 (d, J=9.2 Hz, 6H), 3.69 (s, 2H), 3.59 (s, 2H), 3.54 (s, 1H), 3.43 (s, 2H), 2.01-1.94 (m, 1H), 0.42 (d, J=6.7 Hz, 2H), 0.25 (d, J=7.7 Hz, 2H).

An analogous method was followed to obtain the following compounds.

| | Starting | |
|---|---|---|
| Compounds | Material | Characterization |
| 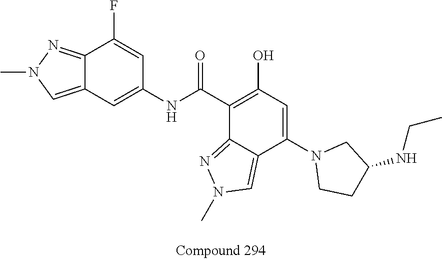 [SMILES: CCN[C@@H]1CCN(c2cc(O)c(C(=O)Nc3cc(F)c4nn(C)cc4c3)c3nn(C)cc23)C1] | 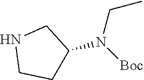 | The resulting mixture was purified by reverse flash chromatography (Condition 5, Gradient 1) to afford 4- [(3R)-3-(ethylamino)-pyrrolidin-1-yl]- N-(7-fluoro-2-methyl-indazol-5-yl)-6- hydroxy-2-methyl-indazole-7- carboxamide (Compound 294, 30 mg, 66.5 mol, 38% yield) as a solid. LCMS (ES, m/z): 452 [M + H]⁺. ¹H NMR (400 MHz, DMSO-d₆) δ 13.77 (s, 1H), 11.14 (s, 1H), 8.66 (s, 1H), 8.42 (d, J = 2.8 Hz, 1H), 7.93 (d, J = 1.6 Hz, 1H), 7.40-7.32 (m, 1H), 5.48 (s, 1H), 4.18 (d, J = 9.1 Hz, 6H), 3.74 (s, 2H), 3.68 (s, 1H), 3.59 (s, 1H), 3.47-3.40 (m, 1H), 3.36 (s, 1H), 2.62 (q, J = 7.1 Hz, 2H), 2.14 (dt, J = 13.1, 6.3 Hz, 1H), 1.93-1.86 (m, 1H), 1.04 (t, J = 7.1 Hz, 3H). |

### Example 90: Synthesis of Compound 333

### Synthesis of B73

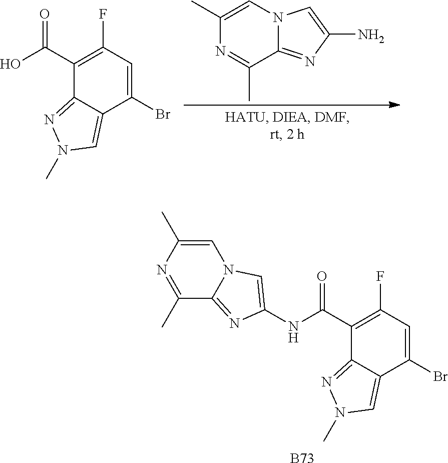 [SMILES: Cn1cc2c(Br)cc(F)c(C(=O)O)c2n1 ; Cc1cn2cc(N)nc2c(C)n1 ; "B73" ; Cc1cn2cc(NC(=O)c3c(F)cc(Br)c4cn(C)nc34)nc2c(C)n1]

To a stirred mixture of 4-bromo-6-fluoro-2-methyl-indazole-7-carboxylic acid (1.1 g, 4.03 mmol) and 6,8-dimethylimidazo[1,2-a]pyrazin-2-amine dihydrochloride (784 mg, 4.83 mmol) in DMF (14 mL) were added DIEA (1.56 g, 12.09 mmol, 2.10 mL) and HATU (2.30 g, 6.04 mmol), and the reaction was stirred for 2 h at room temperature. The resulting mixture was quenched by the addition of water (100 mL). The precipitated solids were collected by filtration, washed with water (30 mL), and dried under infrared light to afford 4-bromo-N-(6,8-dimethylimidazo[1,2-a]pyrazin-2-yl)-6-fluoro-2-methyl-indazole-7-carboxamide (B73, 1.5 g, 3.60 mmol, 89% yield) as a solid. LCMS (ES, m/z): 417 [M+H]⁺.

### Synthesis of B74

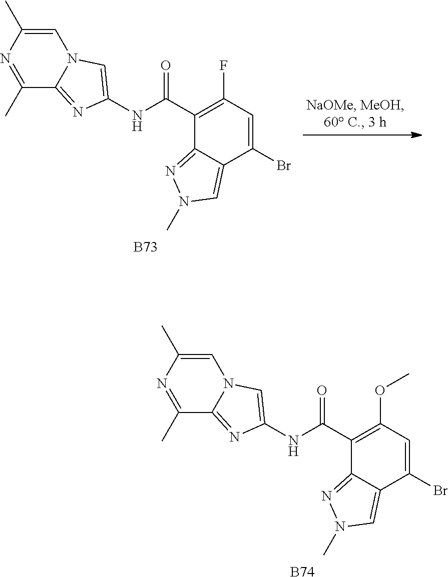 [SMILES: "B73" ; Cc1cn2cc(NC(=O)c3c(F)cc(Br)c4cn(C)nc34)nc2c(C)n1 ; "B74" ; COc1cc(Br)c2cn(C)nc2c1C(=O)Nc1cn2cc(C)nc(C)c2n1]

A solution of 4-bromo-N-(6,8-dimethylimidazo[1,2-a]pyrazin-2-yl)-6-fluoro-2-methyl-indazole-7-carboxamide (370 mg, 886.8 μmol) in MeOH (5 mL) was treated with NaOMe (239 mg, 4.43 mmol) at rt. The resulting mixture was stirred for 3 h at 60° C. under N₂. The mixture was allowed to cool down to room temperature. The reaction was quenched by the addition of water (40 mL) at room temperature. The resulting mixture was extracted with EtOAc (3×40 mL). The combined organic layers were washed with water (2×50 mL) and brine (50 mL), anhydrous Na₂SO₄, and filtered. The filtrate was concentrated under reduced pressure, to afford 4-bromo-N-(6,8-dimethylimidazo[1,2-a]pyrazin-2-yl)-6-methoxy-2-methyl-indazole-7-carboxamide (380 mg, 885.2 μmol, 99.8% yield) as a solid. LCMS (ES, m/z): 429 [M+H]⁺.

### Synthesis of B75

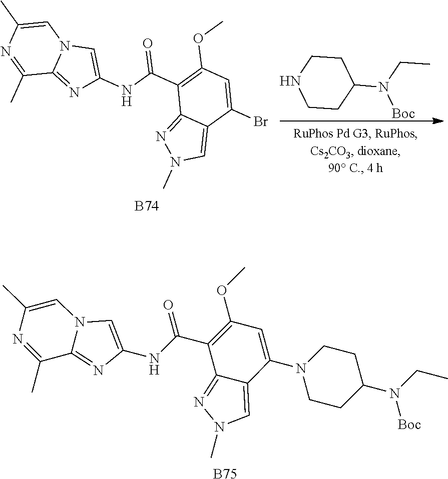

To a stirred mixture of 4-bromo-N-(6,8-dimethylimidazo[1,2-a]pyrazin-2-yl)-6-methoxy-2-methyl-indazole-7-carboxamide (360 mg, 838.6 μmol) and tert-butyl N-ethyl-N-(4-piperidyl)carbamate (248 mg, 1.09 mmol) in dioxane (7 mL) were added Cs₂CO₃ (819 mg, 2.52 mmol), RuPhos (78 mg, 167.7 μmol) and RuPhos Pd G3 (70 mg, 83.86 μmol) at room temperature under N₂. The resulting mixture was stirred for 4 h at 90° C. under N₂. The mixture was allowed to cool down to room temperature, diluted with water (30 mL), and extracted with EtOAc (2×20 mL). The combined organic layers were washed with brine (30 mL), dried over anhydrous Na₂SO₄, and filtered. The filtrate was concentrated under reduced pressure and purified by silica gel column chromatography (75% EtOAc in PE) to afford tert-butyl N-[1-[7-[(6,8-dimethylimidazo[1,2-a]pyrazin-2-yl)carbamoyl]-6-methoxy-2-methyl-indazol-4-yl]-4-piperidyl]-N-ethyl-carbamate (B75, 200 mg, 346.8 μmol, 41% yield) as a solid. LCMS (ES, m/z): 577 [M+H]⁺.

### Synthesis of Compound 333

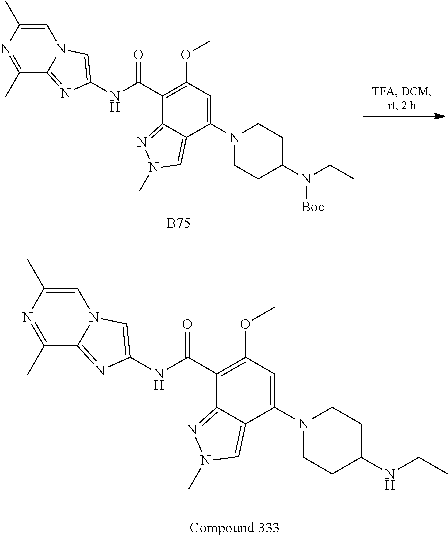

To a stirred mixture of tert-butyl N-[1-[7-[(6,8-dimethylimidazo[1,2-a]pyrazin-2-yl)-carbamoyl]-6-methoxy-2-methyl-indazol-4-yl]-4-piperidyl]-N-ethyl-carbamate (60 mg, 104.0 μmol) in DCM (2 mL) was added TFA (0.5 mL) dropwise, and the reaction was stirred for 2 h at room temperature. The resulting mixture was concentrated under reduced pressure and purified by Prep-HPLC (Condition 14, Gradient 2) to afford N-(6,8-dimethylimidazo[1,2-a]pyrazin-2-yl)-4-[4-(ethylamino)-1-piperidyl]-6-methoxy-2-methyl-indazole-7-carboxamide (Compound 333, 22 mg, 46.16 μmol, 44% yield) as a solid. LCMS (ES, m/z): 477 [M+H]⁺. ¹H NMR (400 MHz, DMSO-d₆) δ 11.44 (s, 1H), 8.60 (s, 1H), 8.29 (d, J=3.1 Hz, 2H), 6.21 (s, 1H), 4.17 (s, 3H), 3.90 (s, 5H), 3.12-2.96 (m, 2H), 2.67 (s, 4H), 2.61 (q, J=7.1 Hz, 2H), 2.41-2.35 (m, 3H), 2.05-1.88 (m, 2H), 1.44 (q, J=9.6 Hz, 2H), 1.04 (t, J=7.1 Hz, 3H).

### Example 91: Synthesis of Compound 332

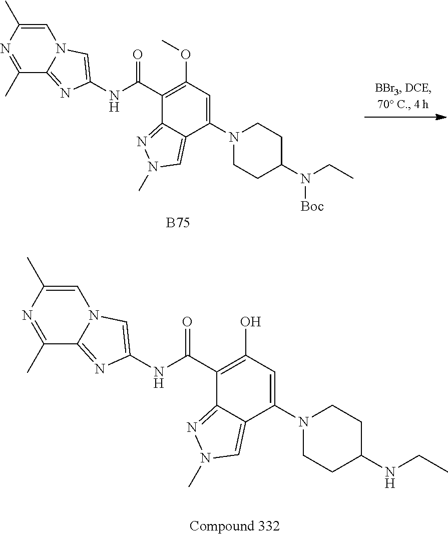

A solution of tert-butyl N-[1-[7-[(6,8-dimethylimidazo[1,2-a]pyrazin-2-yl)carbamoyl]-6-methoxy-2-methyl-indazol-4-yl]-4-piperidyl]-N-ethyl-carbamate (90 mg, 156.1 μmol) in DCE (2 mL) was treated with BBr₃ (0.5 mL), and the reaction was stirred for 4 h at 70° C. under N₂. The resulting mixture was concentrated under reduced pressure and purified by Prep-HPLC (Condition 2, Gradient 3) to afford N-(6,8-dimethylimidazo[1,2-a]pyrazin-2-yl)-4-(4-(ethylamino)piperidin-1-yl)-6-hydroxy-2-methyl-2H-indazole-7-carboxamide 2,2,2-trifluoroacetate (Compound 332, 33.4 mg, 57.9 μmol, 37% yield) as a solid. LCMS (ES, m/z): 463 [M+H]⁺. ¹H NMR (400 MHz, DMSO-d₆) δ 13.55 (s, 1H), 11.53 (s, 1H), 8.74 (s, 1H), 8.48 (s, 2H), 8.35 (d, J=13.5 Hz, 2H), 6.04 (s, 1H), 4.21 (s, 3H), 4.07 (d, J=13.2 Hz, 2H), 3.37 (s, 1H), 3.06 (q, J=11.9, 10.9 Hz, 4H), 2.74 (s, 3H), 2.40 (d, J=1.0 Hz, 3H), 2.12 (d, J=11.9 Hz, 2H), 1.82-1.52 (m, 2H), 1.22 (t, J=7.2 Hz, 3H).

### Example 92: Synthesis of Compound 434

### Synthesis of B76

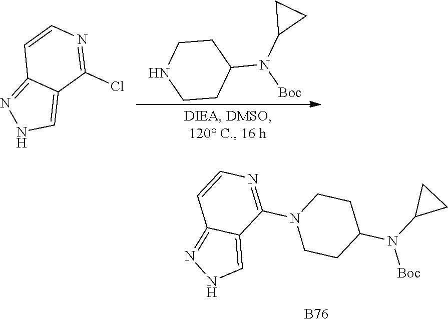

To a solution of 4-chloro-2H-pyrazolo[4,3-c]pyridine (1.9 g, 12.37 mmol) in DMSO (20 mL) were added DIEA (2.40 g, 18.56 mmol, 3.23 mL) and tert-butyl cyclopropyl(piperidin-4-yl) carbamate (3.56 g, 14.84 mmol), and the reaction was stirred for 16 h at 120° C. The reaction was diluted with H₂O (80 mL) and extracted with EtOAc (2×80 mL). The combined organic layers were washed with H₂O (2×100 mL), dried over anhydrous Na₂SO₄, and filtered. The filtrate was concentrated under reduced pressure and purified by silica gel column chromatography (20% EtOAc in DCM) to afford tert-butyl N-cyclopropyl-N-[1-(2H-pyrazolo[4,3-c]pyridin-4-yl)-4-piperidyl]carbamate (B76, 2.8 g, 7.83 mmol, 63% yield) as an oil. LCMS (ES, m/z): 358 [M+H]⁺.

### Synthesis of B77

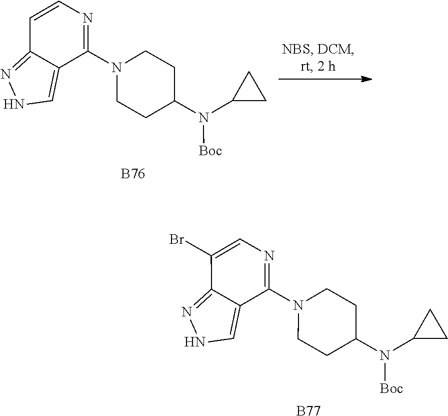 [SMILES: "B76" ; CC(C)(C)OC(=O)N(C1CC1)C1CCN(c2nccc3n[nH]cc23)CC1 ; "B77" ; CC(C)(C)OC(=O)N(C1CC1)C1CCN(c2ncc(Br)c3n[nH]cc23)CC1]

To a solution of tert-butyl N-cyclopropyl-N-[1-(2H-pyrazolo[4,3-c]pyridin-4-yl)-4-piperidyl]carbamate (2.83 g, 7.92 mmol) in DCM (30 mL) was added NBS (1.41 g, 7.92 mmol), and the reaction was stirred for 2 h at rt. The reaction was washed with H₂O (2×40 mL), dried over anhydrous Na₂SO₄, and filtered. The filtrate was concentrated under reduced pressure and purified by silica gel column chromatography (67% EtOAc in PE) to afford tert-butyl N-[1-(7-bromo-2H-pyrazolo[4,3-c]pyridin-4-yl)-4-piperidyl]-N-cyclopropyl-carbamate (B77, 2.15 g, 4.93 mmol, 62% yield) as an oil. LCMS (ES, m/z): 436 [M+H]⁻.

### Synthesis of B78

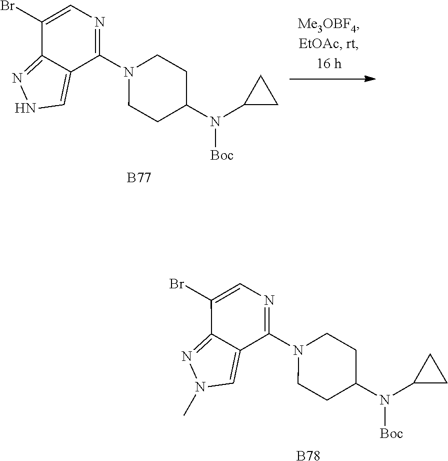 [SMILES: "B77" ; CC(C)(C)OC(=O)N(C1CC1)C1CCN(c2ncc(Br)c3n[nH]cc23)CC1 ; "B78" ; Cn1cc2c(N3CCC(N(C(=O)OC(C)(C)C)C4CC4)CC3)ncc(Br)c2n1]

To a solution of tert-butyl N-[1-(7-bromo-2H-pyrazolo[4,3-c]pyridin-4-yl)-4-piperidyl]-N-cyclopropyl-carbamate (2.05 g, 4.7 mmol) in EtOAc (20 mL) was added Me₃OBF₄ (833.8 mg, 5.64 mmol), and the reaction was stirred for 16 h at rt. The resulting mixture was diluted with water, adjusted to pH 8 with NaHCO₃ (aq.), and extracted with DCM (3×100 mL). The combined organic layers were washed with H₂O (2×200 mL), dried over anhydrous Na₂SO₄, and filtered. The filtrate was concentrated under reduced pressure and purified by silica gel column chromatography (11% EtOAc in DCM) to afford tert-butyl N-[1-(7-bromo-2-methyl-pyrazolo[4,3-c]pyridin-4-yl)-4-piperidyl]-N-cyclopropyl-carbamate (B78, 1.4 g, 3.11 mmol, 66% yield) as a solid. LCMS (ES, m/z): 450 [M+H]⁺.

### Synthesis of B79

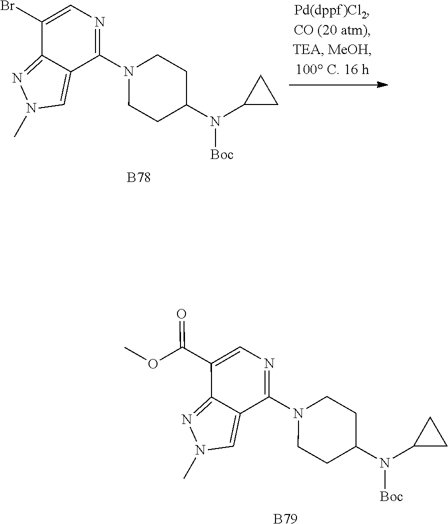 [SMILES: "B78" ; Cn1cc2c(N3CCC(N(C(=O)OC(C)(C)C)C4CC4)CC3)ncc(Br)c2n1 ; "B79" ; COC(=O)c1cnc(N2CCC(N(C(=O)OC(C)(C)C)C3CC3)CC2)c2cn(C)nc12]

To a solution of tert-butyl N-[1-(7-bromo-2-methyl-pyrazolo[4,3-c]pyridin-4-yl)-4-piperidyl]-N-cyclopropyl-carbamate (1.3 g, 2.89 mmol) in MeOH (30 mL) were added TEA (1.46 g, 14.43 mmol) and Pd(dppf)Cl₂ (235.7 mg, 288.7 μmol) in a pressure tank. The mixture was pressurized to 20 atm with carbon monoxide at 100° C. for 16 h. The reaction mixture was cooled to room temperature and filtered to remove insoluble solids. The resulting mixture was concentrated under reduced pressure and purified by silica gel column chromatography (33% EtOAc in DCM) to afford methyl 4-[4-[tert-butoxycarbonyl(cyclopropyl)amino]-1-piperidyl]-2-methyl-pyrazolo[4,3-c]pyridine-7-carboxylate (B79, 1 g, 2.33 mmol, 81% yield) as a solid. LCMS (ES, m/z): 430 [M+H]⁺.

### Synthesis of B80

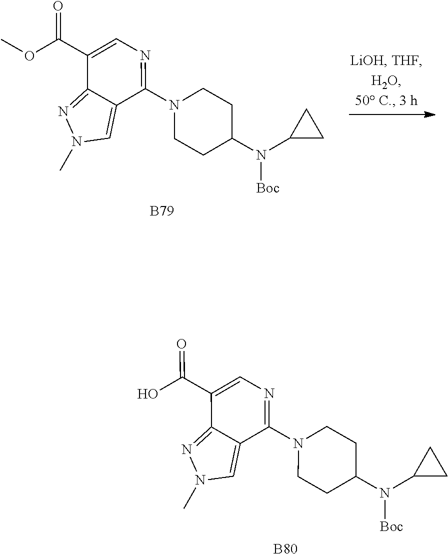 [SMILES: "B79" ; COC(=O)c1cnc(N2CCC(N(C(=O)OC(C)(C)C)C3CC3)CC2)c2cn(C)nc12 ; "B80" ; Cn1cc2c(N3CCC(N(C(=O)OC(C)(C)C)C4CC4)CC3)ncc(C(=O)O)c2n1]

To a solution of methyl 4-[4-[tert-butoxycarbonyl(cyclopropyl)amino]-1-piperidyl]-2-methyl-pyrazolo[4,3-c]pyridine-7-carboxylate (950 mg, 2.21 mmol) in THF (10 mL) and H₂O (5 mL), was added LiOH·H₂O (464.0 mg, 11.06 mmol), and the reaction was stirred for 3 h at 50° C. The resulting mixture was diluted with water (20 mL), adjusted to pH 5-6 with 1 N of HCl (aq.), and extracted with DCM (2×25 mL). The combined organic layers were washed with H₂O (2×50 mL), dried over anhydrous Na₂SO₄, and filtered. The filtrate was concentrated under reduced pressure to afford 4-[4-[tert-butoxycarbonyl-(cyclopropyl)amino]-1-piperidyl]-2-methyl-pyrazolo[4,3-c]pyridine-7-carboxylic acid (860 mg, 2.07 mmol, 94% yield) as a solid. LCMS (ES, m/z): 416 [M+H]⁺.

### Synthesis of B81

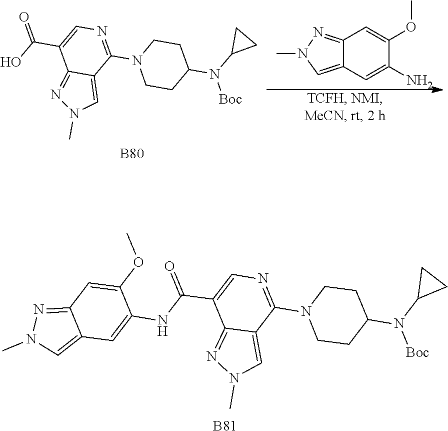

To a solution of 4-[4-[tert-butoxycarbonyl(cyclopropyl)amino]-1-piperidyl]-2-methyl-pyrazolo[4,3-c]pyridine-7-carboxylic acid (100 mg, 240.7 μmol) in MeCN (1.5 mL) were added 6-methoxy-2-methyl-indazol-5-amine (46.9 mg, 264.8 μmol), NMI (79.0 mg, 962.7 μmol, 76.37 μL) and TCFH (101.3 mg, 361.0 μmol), and the reaction was stirred for 2 h at rt. The reaction was diluted with H₂O (5 mL) and the precipitated solids were collected by filtration and further purified by trituration with MTBE (5 mL) to afford tert-butyl N-cyclopropyl-N-[1-[7-[(6-methoxy-2-methyl-indazol-5-yl)carbamoyl]-2-methyl-pyrazolo[4,3-c]pyridin-4-yl]-4-piperidyl]carbamate (130 mg, 226.2 μmol, 94% yield) as a solid. LCMS (ES, m/z): 575 [M+H]⁺.

### Synthesis of Compound 434

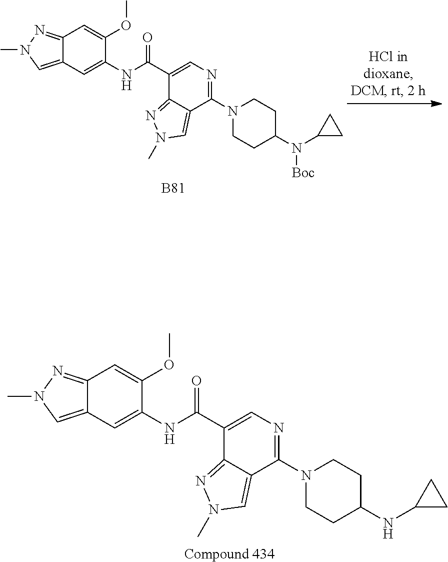

To a solution of tert-butyl N-cyclopropyl-N-[1-[7-[(6-methoxy-2-methyl-indazol-5-yl)-carbamoyl]-2-methyl-pyrazolo[4,3-c]pyridin-4-yl]-4-piperidyl]carbamate (120 mg, 208.8 μmol) in DCM (1.5 mL) was added HCl (4.0 M in 1,4-dioxane) (4 M, 500 μL), and the reaction was stirred for 2 h at rt. The resulting mixture was concentrated under reduced pressure and acidified to pH 8 with saturated NaHCO₃ (aq.) and the precipitated solids were collected by filtration and purified by trituration with MTBE (5 mL) to afford 4-[4-(cyclopropylamino)-1-piperidyl]-N-(6-methoxy-2-methyl-indazol-5-yl)-2-methyl-pyrazolo[4,3-c]pyridine-7-carboxamide (80 mg, 168.6 μmol, 81% yield) as a solid. LCMS (ES, m/z): 475 [M+H]⁺. ¹H NMR (400 MHz, DMSO-d₆) δ 11.15 (s, 1H), 8.95 (s, 1H), 8.78 (s, 1H), 8.46 (s, 1H), 8.16 (s, 1H), 7.04 (s, 1H), 4.51 (dt, J=11.9, 4.0 Hz, 2H), 4.23 (s, 3H), 4.06 (s, 3H), 4.01 (s, 3H), 3.38-3.30 (m, 2H), 2.86 (dt, J=9.6, 4.8 Hz, 1H), 2.25 (s, 1H), 2.10 (tt, J=6.7, 3.6 Hz, 1H), 1.97 (dd, J=13.4, 3.7 Hz, 2H), 1.40-1.25 (m, 2H), 0.42-0.33 (m, 2H), 0.25-0.17 (m, 2H).

An analogous method was followed to obtain the following compounds.

| | Starting | |
|---|---|---|
| Compound | Material | Characterization |
| 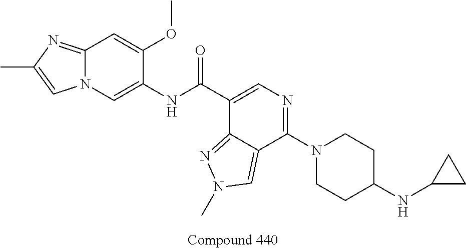 [SMILES: COc1cc2nc(C)cn2cc1NC(=O)c1cnc(N2CCC(NC3CC3)CC2)c2cn(C)nc12] | 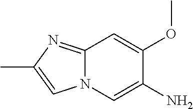 | The precipitated solids were collected by filtration and purified by trituration with MTBE (2 mL) to afford 4-[4- (cyclopropylamino)- 1-piperidyl]-N- (7-methoxy-2- methyl-imidazo[1,2- a]-pyridin-6- yl)-2-methyl- pyrazolo[4,3-c]- pyridine-7- carboxamide (Compound 440, 30 mg, 63.2 μmol, 56% yield) as a solid. LCMS (ES, m/z): 475 [M + H]⁺. ¹H NMR (400 MHz, DMSO-d₆) δ 11.00 (s, 1H), 9.50 (s, 1H), 8.96 (s, 1H), 8.46 (s, 1H), 7.53 (s, 1H), 6.93 (s, 1H), 4.51 (dt, J = 13.7, 4.1 Hz, 2H), 4.22 (s, 3H), 4.03 (s, 3H), 3.39-3.32 (m, 2H), 2.87 (tt, J = 9.4, 4.0 Hz, 1H), 2.24 (s, 3H), 2.11 (tt, J = 6.7, 3.6 Hz, 1H), 2.01-1.93 (m, 2H), 1.40-1.26 (m, 2H), 0.38 (dt, J = 6.2, 3.1 Hz, 2H), 0.21 (p, J = 3.9 Hz, 2H). |
| 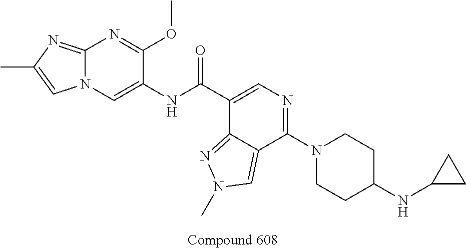 [SMILES: COc1nc2nc(C)cn2cc1NC(=O)c1cnc(N2CCC(NC3CC3)CC2)c2cn(C)nc12] | 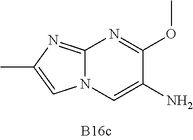 | The precipitated solids were collected by filtration and purified by trituration with MeOH to afford 4-[4- (cyclopropylamino)- 1-piperidyl]-N- (7-methoxy-2- methyl-imidazo[1,2- a]-pyrimidin-6- yl)-2-methyl- pyrazolo-[4,3- c]pyridine-7- carboxamide (Compound 608, 50 mg, 105.1 μmol, 76% yield) as a solid. LCMS (ES, m/z): 476 [M + H]⁺. ¹H NMR (400 MHz, DMSO-d₆) δ 10.96 (s, 1H), 9.69 (s, 1H), 8.98 (s, 1H), 8.46 (s, 1H), 7.48 (s, 1H), 4.51 (d, J = 13.2 Hz, 2H), 4.22 (s, 3H), 4.12 (s, 3H), 3.36 (d, J = 12.3 Hz, 2H), 2.94-2.80 (m, 1H), 2.24 (s, 3H), 2.10 (dt, J = 6.8, 3.3 Hz, 1H), 1.97 (d, J = 12.8 Hz, 2H), 1.33 (q, J = 11.3 Hz, 2H), 0.37 (dt, J = 6.3, 3.0 Hz, 2H), 0.21 (g, J = 3.4, 2.9 Hz, 2H). |
| 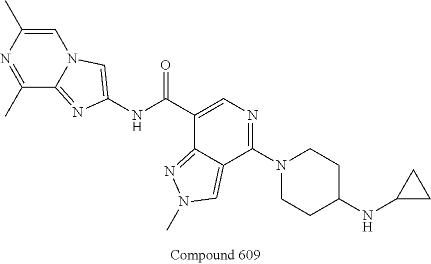 [SMILES: Cc1cn2cc(NC(=O)c3cnc(N4CCC(NC5CC5)CC4)c4cn(C)nc34)nc2c(C)n1] | 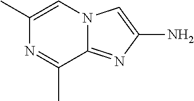 | The precipitated solids were collected by filtration and purified by trituration with MTBE (2 mL) to afford 4-[4- (cyclopropylamino)- 1-piperidyl]-N- (6,8-dimethylimidazo [1,2-a]pyrazin- 2-yl)-2-methyl- pyrazolo[4,3- c]pyridine-7- carboxamide (Compound 609, 16 mg, 34.8 μmol, 35% yield) as a solid. LCMS (ES, m/z): 460 [M + H]⁺. ¹H NMR (400 MHz, DMSO-d₆) δ 10.94 (s, 1H), 9.00 (s, 1H), 8.50 (s, 1H), 8.28 (d, J = 8.8 Hz, 2H), 4.52 (d, J = 13.3 Hz, 2H), 4.25 (s, 3H), 3.41- 3.31 (m, 2H), 2.87 (s, 1H), 2.67 (s, 3H), 2.37-2.33 (m, 3H), 2.26-2.25 (m, 1H), 2.10 (tt, J = 6.7, 3.5 Hz, 1H), 1.97 (d, J = 13.0 Hz, 2H), 1.34 (q, J = 11.3, 9.3 Hz, 2H), 0.38 (td, J = 6.3, 4.0 Hz, 2H), 0.24- 0.16 (m, 2H). |
| 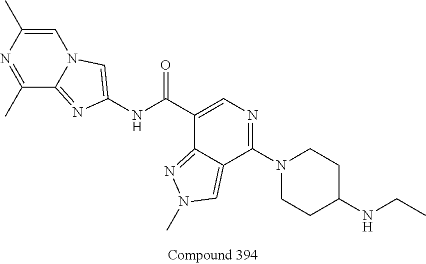 [SMILES: CCNC1CCN(c2ncc(C(=O)Nc3cn4cc(C)nc(C)c4n3)c3nn(C)cc23)CC1] | 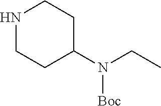 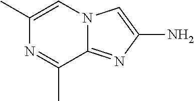 | Modification: Step 2 was ran for 16 h. Step 3 was ran for 1 h. Step 5 was ran for 2 h at room temperature. The resulting mixture was purified by reversed-phase flash chromatography (Condition 3, Gradient 26) to afford N-(6,8- dimethylimidazo[1,2- a]pyrazin-2-yl)-4- [4-(ethylamino)-1- piperidyl]-2-methyl- pyrazolo-[4,3- c]-pyridine-7- carboxamide (Compound 394, 0.017 g, 37.3 μmol, 14% yield) as a solid. LCMS (ES, m/z): 448 [M + H]⁺. ¹H NMR (400 MHz, DMSO-d₆) δ 10.97 (s, 1H), 9.02 (s, 1H), 8.52 (s, 1H), 8.31 (d, J = 9.0 Hz, 2H), 4.55 (d, J = 13.4 Hz, 2H), 4.27 (s, 3H), 2.82- 2.74 (m, 1H), 2.69 (s, 3H), 2.60 (q, J = 7.1 Hz, 3H), 2.38 (d, J = 1.0 Hz, 3H), 1.96 (d, J = 12.4 Hz, 2H), 1.30 (dt, J = 23.7, 11.3 Hz, 2H), 1.03 (t, J = 7.1 Hz, 3H). |

### Example 93: Synthesis of C39

### Synthesis of C37

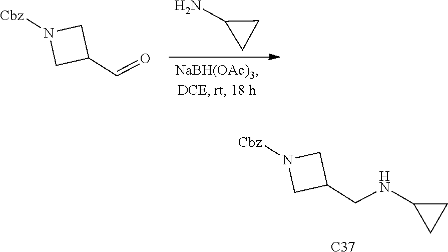

To a stirred solution of benzyl 3-formylazetidine-1-carboxylate (500 mg, 2.28 mmol, 1 eq) in DCE (10 mL) was added cyclopropanamine (260 mg, 4.56 mmol, 2 eq), and the reaction was stirred for 1 h at room temperature. To the above mixture was added NaBH(OAc)₃ (1.45 g, 6.84 mmol, 3 eq), and the reaction was stirred for an additional 17 h at room temperature. The reaction was quenched with water (20 mL) and extracted with DCM (2×30 mL). The combined organic layers were washed with brine (30 mL), dried over anhydrous Na₂SO₄, and filtered. The filtrate was concentrated under reduced pressure, to afford benzyl 3-[(cyclopropylamino)methyl]azetidine-1-carboxylate (600 mg, crude) as an oil. The crude product was used in the next step directly without further purification. LCMS (ES, m/z): 261 [M+H]⁺. Synthesis of C38

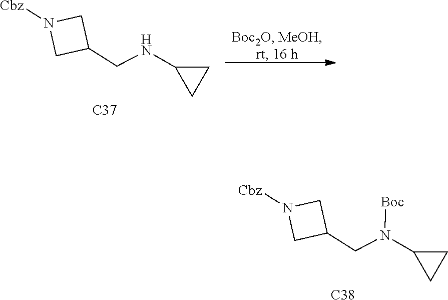 [SMILES: O=C(OCc1ccccc1)N1CC(CNC2CC2)C1 ; "C37" ; CC(C)(C)OC(=O)N(CC1CN(C(=O)OCc2ccccc2)C1)C1CC1 ; "C38"]

To a stirred solution of benzyl 3-[(cyclopropylamino)methyl]azetidine-1-carboxylate (590 mg, 2.3 mmol, 1 eq) in MeOH (6 mL) was added Boc₂O (1.48 g, 6.8 mmol, 3 eq). The resulting mixture was stirred for 16 h at room temperature. The resulting mixture was concentrated under reduced pressure and purified by silica gel column chromatography (25% THF in PE) to afford benzyl 3-[[tert-butoxycarbonyl(cyclopropyl)amino]methyl]azetidine-1-carboxylate (C38, 450 mg, 55% yield) as a solid. LCMS (ES, m/z): 361 [M+H]⁺.

### Synthesis of C39

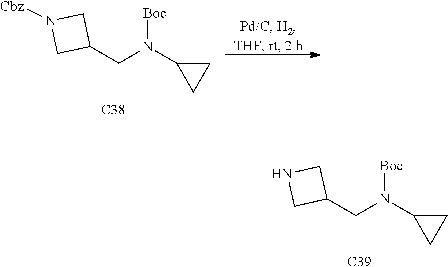 [SMILES: CC(C)(C)OC(=O)N(CC1CN(C(=O)OCc2ccccc2)C1)C1CC1 ; "C38" ; CC(C)(C)OC(=O)N(CC1CNC1)C1CC1 ; "C39"]

To a stirred solution of benzyl 3-[[tert-butoxycarbonyl(cyclopropyl)amino]-methyl]-azetidine-1-carboxylate (200 mg, 554.9 μmol, 1 eq) in THF (2 mL) was added Pd/C (10% on Carbon (wetted with ca. 55% water)) (100 mg, 939.7 μmol). The resulting mixture was stirred for 2 h at room temperature under hydrogen atmosphere. The resulting mixture was filtered, the filter cake was washed with DCM (2×10 mL). The filtrate was concentrated under reduced pressure to afford tert-butyl N-(azetidin-3-ylmethyl)-N-cyclopropyl-carbamate (160 mg, crude) as an oil. LCMS (ES, m/z): 227 [M+H]⁺.

### Example 94: Synthesis of Compounds 307, 303

### Synthesis of B82

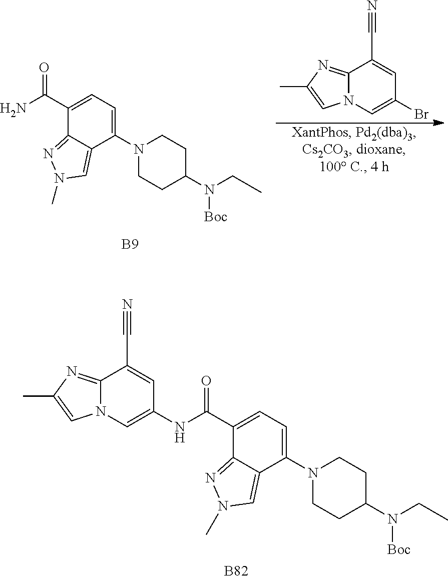

To a stirred solution of tert-butyl N-[1-(7-carbamoyl-2-methyl-indazol-4-yl)-4-piperidyl]-N-ethyl-carbamate (100 mg, 249.1 μmol, 1 eq) and 6-bromo-2-methyl-imidazo[1,2-a]pyridine-8-carbonitrile (64 mg, 274 μmol, 1.1 eq) in dioxane (2 mL) were added Cs₂CO₃ (243 mg, 747.2 μmol, 3 eq), Xantphos (28 mg, 49.81 μmol, 0.2 eq) and Pd₂(dba)₃ (22 mg, 24.91 μmol. 0.1 eq) at room temperature under N₂. The resulting mixture was stirred for 4 h at 100° C. under N₂. The resulting mixture was concentrated under reduced pressure and purified by silica gel column chromatography (5% MeOH in DCM) to afford tert-butyl N-[1-[7-[(8-cyano-2-methyl-imidazo[1,2-a]pyridin-6-yl)carbamoyl]-2-methyl-indazol-4-yl]-4-piperidyl]-N-ethyl-carbamate (B82, 76 mg, 55% yield) as a solid. LCMS (ES, m/z): 557 [M+H]⁺.

### Synthesis of Compound 307

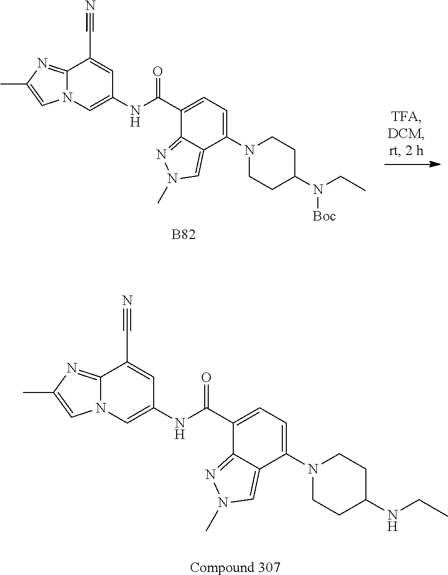

To a stirred solution of tert-butyl N-[1-[7-[(8-cyano-2-methyl-imidazo[1,2-a]pyridin-6-yl)carbamoyl]-2-methyl-indazol-4-yl]-4-piperidyl]-N-ethyl-carbamate (71 mg, 127.6 μmol, 1 eq) in DCM (2 mL) was added TFA (1 mL), and the reaction was stirred for 2 h at room temperature. The resulting mixture was concentrated under reduced pressure and purified by Prep-HPLC (Condition 14, Gradient 1) to afford N-(8-cyano-2-methyl-imidazo[1,2-a]pyridin-6-yl)-4-[4-(ethylamino)-1-piperidyl]-2-methyl-indazole-7-carboxamide (Compound 307, 29.2 mg, 49% yield) as a solid. LCMS (ES, m/z): 457 [M+H]⁺. ¹H NMR (300 MHz, DMSO-d₆) δ 11.16 (s, 1H), 9.64 (d, J=2.0 Hz, 1H), 8.78 (s, 1H), 8.10 (d, J=2.0 Hz, 1H), 7.98 (t, J=4.1 Hz, 2H), 6.49 (d, J=8.2 Hz, 1H), 4.31 (s, 3H), 3.90 (d, J=12.7 Hz, 2H), 3.08 (t, J=11.8 Hz, 2H), 2.72-2.55 (m, 3H), 2.39 (s, 3H), 1.96 (d, J=12.6 Hz, 2H), 1.45 (t, J=10.6 Hz, 2H), 1.04 (t, J=7.1 Hz, 3H).

An analogous method was followed to obtain the following compounds.

| | Starting | |
|---|---|---|
| Compound | Material | Characterization |
| 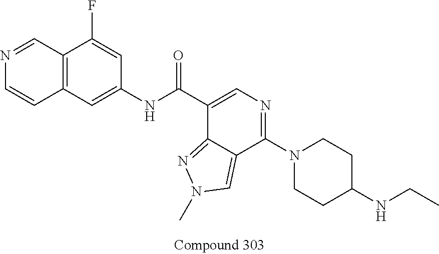 [SMILES: CCNC1CCN(c2ncc(C(=O)Nc3cc(F)c4cnccc4c3)c3nn(C)cc23)CC1] | 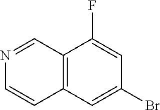 [SMILES: Fc1cc(Br)cc2ccncc12] | The crude product was purified by Prep- HPLC (Condition 14, Gradient 1) to afford 4-[4-(ethylamino)- 1-piperidyl]-N- (8-fluoro-6- isoquinolyl)-2- methylindazole- 7-carboxamide (Compound 303, 34 mg, 69% yield) as a solid. LCMS (ES, m/z): 447 [M + H]⁺. ¹H NMR (400 MHz, DMSO-d₆) δ 11.61 (s, 1H), 9.34 (s, 1H), 8.80 (s, 1H), 8.54 (d, J = 5.8 Hz, 1H), 8.29 (s, 1H), 8.02 (d, J = 8.1 Hz, 1H), 7.96 (d, J = 12.5 Hz, 1H), 7.86 (d, J = 5.8 Hz, 1H), 6.51 (d, J = 8.2 Hz, 1H), 4.33 (s, 3H), 3.92 (d, J = 12.6 Hz, 2H), 3.10 (t, J = 11.9 Hz, 2H), 2.69 (s, 1H), 2.61 (q, J = 7.1 Hz, 2H), 1.96 (d, J = 12.7 Hz, 2H), 1.61 (s, 1H), 1.44 (q, J = 11.4, 10.8 Hz, 2H), 1.04 (t, J = 7.1 Hz, 3H). |
| 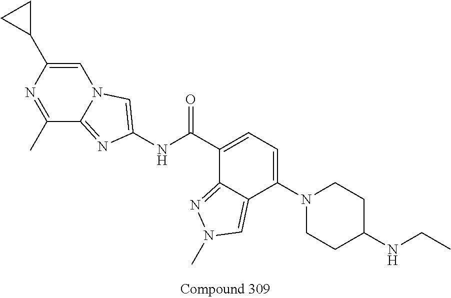 [SMILES: CCNC1CCN(c2ccc(C(=O)Nc3cn4cc(C5CC5)nc(C)c4n3)c3nn(C)cc23)CC1] | 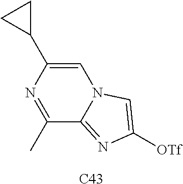 | The crude product was purified by Prep- HPLC (Condition 14, Gradient 1) to afford N-(6- cyclopropyl-8- methylimidazo[1,2-a]- pyrazin-2-yl)-4- [4-(ethylamino)-1- piperidyl]-2-methyl- indazole-7-carboxamide (Compound 309, 20.3 mg, 41% yield) as a solid. LCMS (ES, m/z): 473 [M + H]⁺. ¹H NMR (300 MHz, DMSO- d₆) δ 11.43 (s, 1H), 8.78 (s, 1H), 8.39 (s, 1H), 8.31 (s, 1H), 8.03 (d, J = 8.1 Hz, 1H), 6.50 (d, J = 8.2 Hz, 1H), 4.30 (s, 3H), 3.90 (d, J = 12.7 Hz, 2H), 3.08 (t, J = 11.7 Hz, 2H), 2.78- 2.56 (m, 6H), 2.00 (dd, J = 16.9, 10.0 Hz, 3H), 1.45 (q, J = 11.0 Hz, 2H), 1.05 (t, J = 7.1 Hz, 3H), 0.93-0.84 (m, 4H). |
| 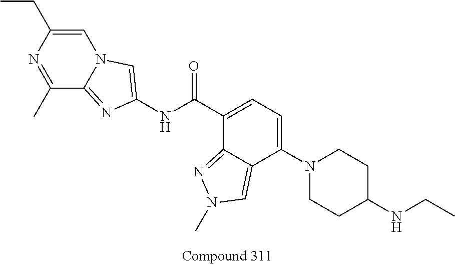 | 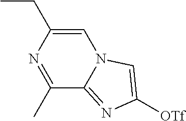 [SMILES: CCc1cn2cc(OS(=O)(=O)C(F)(F)F)nc2c(C)n1] | Modification: Step 1 was ran for 16 h. The residue was purified by Prep-HPLC (Condition 2, Gradient 4) to afford N-(6- ethyl-8-methylimidazo [1,2-a]pyrazin-2- yl)-4-(4-(ethylamino) piperidin-1-yl)-2- methyl-2H-indazole- 7-carboxamide 2,2,2-trifluoroacetate (Compound 311, 0.005 g, 8.7 μmol, 5% yield) as a solid. LCMS (ES, m/z): 392 [M + H]⁺. ¹H NMR (300 MHz, Methanol- d₄) δ 8.59 (d, J = 6.1 Hz, 2H), 8.43 (s, 1H), 8.17 (d, J = 8.1 Hz, 1H), 6.63 (d, J = 8.1 Hz, 1H), 4.39 (s, 3H), 4.17 (d, J = 13.2 Hz, 2H), 3.23-3.10 (m, 5H), 2.93 (s, 3H), 2.86 (q, J = 7.7 Hz, 2H), 2.28 (d, J = 12.0 Hz, 2H), 1.86 (d, J = 12.0 Hz, 2H), 1.45-1.32 (m, 6H). |
| 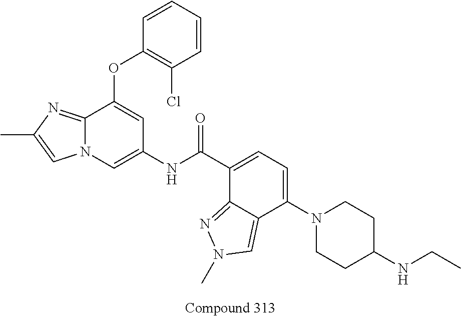 [SMILES: CCNC1CCN(c2ccc(C(=O)Nc3cc(Oc4ccccc4Cl)c4nc(C)cn4c3)c3nn(C)cc23)CC1] | 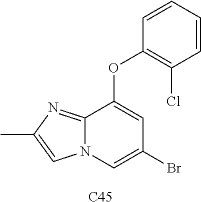 | Modification: The first step was ran at 90° C. The crude product was purified by Prep- HPLC (Condition 6, Gradient 3) to afford N-[8-(2- chlorophenoxy)-2- methylimidazo-[1,2-a] pyridin-6-yl]-4- [4-(ethylamino)-1- piperidyl]-2-methyl- indazole-7-carboxamide (Compound 313, 24 mg, 42 μmol, 40% yield) as a solid. LCMS (ES, m/z): 558 [M + H]⁺. ¹H NMR (400 MHz, DMSO-d₆) δ 10.97 (s, 1H), 9.14 (d, J = 1.7 Hz, 1H), 8.74 (s, 1H), 7.93 (d, J = 8.1 Hz, 1H), 7.86 (s, 1H), 7.66 (dd, J = 8.0, 1.6 Hz, 1H), 7.36 (td, J = 7.7, 1.7 Hz, 1H), 7.25 (td, J = 7.7, 1.5 Hz, 1H), 7.11 (dd, J = 8.1, 1.5 Hz, 1H), 6.85 (d, J = 1.7 Hz, 1H), 6.47 (d, J = 8.2 Hz, 1H), 4.20 (s, 3H), 3.87 (d, J = 12.7 Hz, 2H), 3.10-2.99 (m, 2H), 2.69-2.56 (m, 2H), 2.31 (s, 3H), 1.95 (d, J = 12.4 Hz, 2H), 1.60 (s, 1H), 1.43 (q, J = 9.9 Hz, 2H), 1.04 (t, J = 7.1 Hz, 3H). |
| 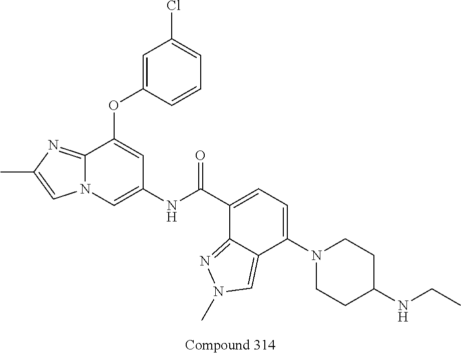 [SMILES: CCNC1CCN(c2ccc(C(=O)Nc3cc(Oc4cccc(Cl)c4)c4nc(C)cn4c3)c3nn(C)cc23)CC1] | 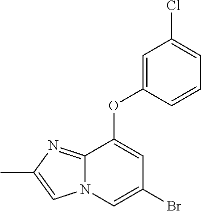 | The crude product was purified by Prep- HPLC (Condition 6, Gradient 3) to afford N-[8-(3- chlorophenoxy)-2- methylimidazo-[1,2-a] pyridin-6-yl]-4- [4-(ethylamino)-1- piperidyl]-2-methyl- indazole-7-carboxamide (Compound 314, 15.3 mg, 27.4 μmol, 26% yield) as a solid. LCMS (ES, m/z): 558 [M + H]⁺. ¹H NMR (400 MHz, DMSO-d₆) δ 11.02 (s, 1H), 9.23 (d, J = 1.7 Hz, 1H), 8.75 (s, 1H), 7.95 (d, J = 8.1 Hz, 1H), 7.86 (d, J = 1.0 Hz, 1H), 7.42 (t, J = 8.2 Hz, 1H), 7.26-7.19 (m, 1H), 7.17 (t, J = 2.2 Hz, 1H), 7.10 (d, J = 1.8 Hz, 1H), 7.05 (dd, J = 8.3, 2.4 Hz, 1H), 6.48 (d, J = 8.2 Hz, 1H), 4.24 (s, 3H), 3.92-3.84 (m, 2H), 3.11-2.99 (m, 2H), 2.65-2.64 (m, 1H), 2.60 (q, J = 7.1 Hz, 2H), 2.29 (s, 3H), 1.96 (d, J = 13.3 Hz, 2H), 1.44-1.40 (m, 2H), 1.04 (t, J = 7.1 Hz, 3H). |
| 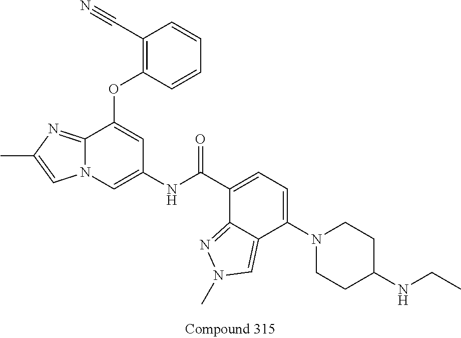 [SMILES: CCNC1CCN(c2ccc(C(=O)Nc3cc(Oc4ccccc4C#N)c4nc(C)cn4c3)c3nn(C)cc23)CC1] | 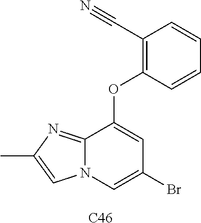 | Modification: The first step was ran at 90° C. for 3 h. The residue was purified by reversed- phase flash chromatography (Condition 3, Gradient 11) to afford N-[8-(2-cyano- phenoxy)-2-methyl- imidazo[1,2- a]pyridin-6-yl]-4- [4-(ethylamino)-1- piperidyl]-2-methyl- indazole-7- carboxamide (Compound 315, 0.006 g, 10.9 μmol, 9% yield) as a solid. LCMS (ES, m/z): 549 [M + H]⁺. ¹H NMR (300 MHz, Methanol-d₄) δ 9.07 (d, J = 1.7 Hz, 1H), 8.42 (s, 1H), 8.01 (d, J = 8.1 Hz, 1H), 7.83 (dd, J = 7.8, 1.7 Hz, 1H), 7.69- 7.55 (m, 2H), 7.30 (t, J = 7.6 Hz, 1H), 7.06 (d, J = 1.7 Hz, 1H), 7.00 (d, J = 8.5 Hz, 1H), 6.47 (d, J = 8.2 Hz, 1H), 4.23 (s, 3H), 3.98 (d, J = 12.7 Hz, 2H), 2.99 (t, J = 12.2 Hz, 2H), 2.76 (q, J = 7.3 Hz, 3H), 2.36 (s, 3H), 2.08 (d, J = 12.5 Hz, 2H), 1.67-1.51 (m, 2H), 1.18 (t, J = 7.2 Hz, 3H). |
| 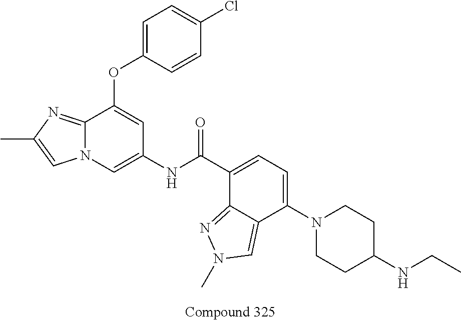 [SMILES: CCNC1CCN(c2ccc(C(=O)Nc3cc(Oc4ccc(Cl)cc4)c4nc(C)cn4c3)c3nn(C)cc23)CC1] | 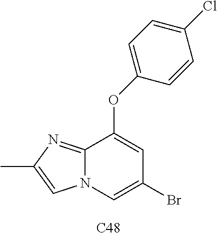 | Modification: Step 1 was ran at 90° C. for 4 h. The residue was purified by reversed- phase flash chromatography (Condition 3, Gradient 11) to afford N-[8-(4-chloro- phenoxy)-2- methylimidazo[1,2-a]- pyridin-6-yl]-4- [4-(ethylamino)-1- piperidyl]-2-methyl- indazole-7- carboxamide (Compound 325, 26 mg, 47.4 μmol, 31% yield) as a solid. LCMS (ES, m/z): 558 [M + H]⁺. ¹H NMR (400 MHz, DMSO-d₆) δ 11.00 (s, 1H), 9.19 (d, J = 1.8 Hz, 1H), 8.75 (s, 1H), 7.95 (d, J = 8.1 Hz, 1H), 7.85 (d, J = 1.1 Hz, 1H), 7.49-7.40 (m, 2H), 7.16-7.08 (m, 2H), 7.05 (d, J = 1.8 Hz, 1H), 6.48 (d, J = 8.3 Hz, 1H), 4.23 (s, 3H), 3.88 (d, J = 12.8 Hz, 2H), 3.05 (t, J = 11.4 Hz, 2H), 2.68 (s, 1H), 2.61 (q, J = 7.1 Hz, 3H), 2.31- 2.26 (m, 3H), 1.96 (d, J = 12.4 Hz, 2H), 1.43 (q, J = 10.2 Hz, 2H), 1.04 (t, J = 7.1 Hz, 3H). |
| 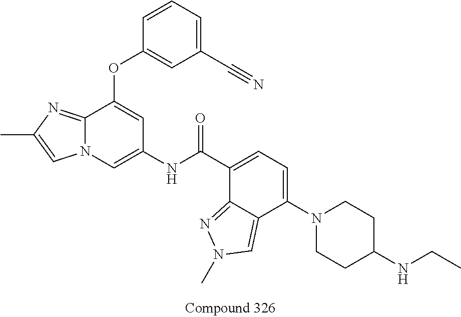 [SMILES: CCNC1CCN(c2ccc(C(=O)Nc3cc(Oc4cccc(C#N)c4)c4nc(C)cn4c3)c3nn(C)cc23)CC1] | 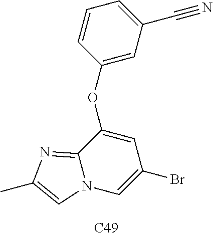 [SMILES: Cc1cn2cc(Br)cc(Oc3cccc(C#N)c3)c2n1 ; "C49"] | Modification: Step 1 was ran at 90° C. for 4 h. The residue was purified by reversed- phase flash chromatography (Condition 3, Gradient 11) to afford N-[8-(3-cyano- phenoxy)-2- methylimidazo[1,2- a]pyridin-6-yl]-4- [4-(ethylamino)-1- piperidyl]-2- methyl-indazole-7- carboxamide (Compound 326, 26 mg, 47.4 μmol, 31% yield) as a solid. LCMS (ES, m/z): 549 [M + H]⁺. ¹H NMR (400 MHz, DMSO-d₆) δ 11.04 (s, 1H), 9.28 (d, J = 1.7 Hz, 1H), 8.76 (s, 1H), 7.96 (d, J = 8.1 Hz, 1H), 7.87 (d, J = 1.0 Hz, 1H), 7.65-7.54 (m, 3H), 7.42 (dt, J = 7.6, 2.1 Hz, 1H), 7.16 (d, J = 1.8 Hz, 1H), 6.48 (d, J = 8.2 Hz, 1H), 4.24 (s, 3H), 3.88 (d, J = 12.6 Hz, 2H), 3.05 (t, J = 11.7 Hz, 2H), 2.61 (q, J = 7.1 Hz, 3H), 2.28 (s, 3H), 1.96 (d, J = 12.8 Hz, 2H), 1.43 (q, J = 10.5 Hz, 2H), 1.04 (t, J = 7.1 Hz, 3H). |
| 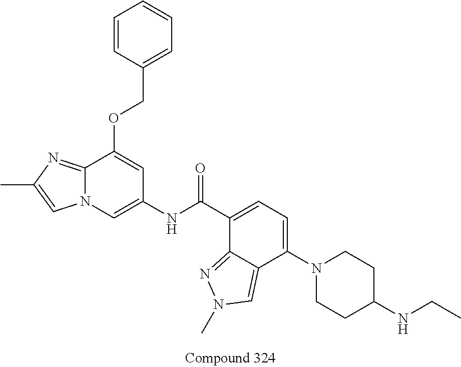 [SMILES: CCNC1CCN(c2ccc(C(=O)Nc3cc(OCc4ccccc4)c4nc(C)cn4c3)c3nn(C)cc23)CC1] | 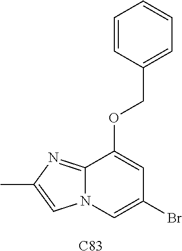 | Modification: Step 1 was ran at 90° C. The residue was purified by reversed- phase flash chromatography (Condition 3, Gradient 11) to afford N-(8- benzyloxy-2- methylimidazo[1,2- a]pyridin-6-yl)-4-[4- (ethylamino)-1- piperidyl]-2- methyl-indazole-7- carboxamide (Compound 324, 25 mg, 46.5 μmol, 25% yield) as a solid. LCMS (ES, m/z): 538 [M + H]⁺. ¹H NMR (400 MHz, DMSO-d₆) δ 10.97 (s, 1H), 8.98 (d, J = 1.6 Hz, 1H), 8.78 (s, 1H), 7.96 (d, J = 8.1 Hz, 1H), 7.73 (s, 1H), 7.57 (d, J = 7.0 Hz, 2H), 7.46 (dd, J = 8.3, 6.6 Hz, 2H), 7.39 (dd, J = 8.5, 6.1 Hz, 1H), 6.74 (d, J = 1.7 Hz, 1H), 6.48 (d, J = 8.2 Hz, 1H), 5.38 (s, 2H), 4.31 (s, 3H), 3.88 (d, J = 12.3 Hz, 2H), 3.05 (t, J = 11.8 Hz, 2H), 2.71-2.57 (m, 3H), 2.31 (s, 3H), 1.96 (d, J = 12.5 Hz, 2H), 1.45 (q, J = 10.8, 10.4 Hz, 2H), 1.05 (t, J = 7.1 Hz, 3H). |
| 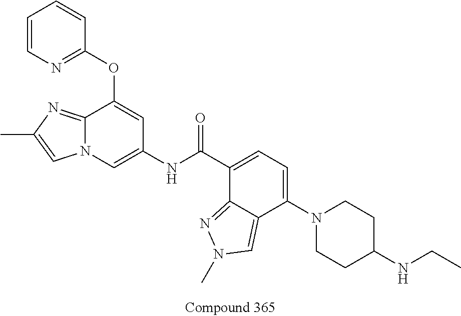 [SMILES: CCNC1CCN(c2ccc(C(=O)Nc3cc(Oc4ccccn4)c4nc(C)cn4c3)c3nn(C)cc23)CC1] | 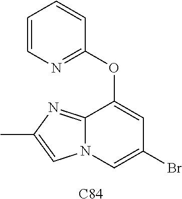 | Modification: Step 1 was ran at 90° C. for 2 h. Step 2 was ran for 1 h. The crude product was purified by Prep- HPLC (Condition 6, Gradient 4) to afford 4-[4-(ethylamino)- 1-piperidyl]-2- methyl-N-[2-methyl- 8-(2-pyridyloxy)- imidazo[1,2-a]pyridin- 6-yl]indazole-7- carboxamide (Compound 365, 8.4 mg, 16 μmol, 17% yield) as a solid. LCMS (ES, m/z): 525 [M + H]⁺. ¹H NMR (400 MHz, DMSO-d₆) δ 11.06 (s, 1H), 9.29 (d, J = 1.8 Hz, 1H), 8.76 (s, 1H), 8.10 (dd, J = 5.5, 1.9 Hz, 1H), 7.97 (d, J = 8.1 Hz, 1H), 7.90 (ddd, J = 9.1, 7.3, 2.0 Hz, 1H), 7.82 (d, J = 1.1 Hz, 1H), 7.25-7.11 (m, 3H), 6.49 (d, J = 8.3 Hz, 1H), 4.26 (s, 3H), 3.88 (d, J = 12.9 Hz, 2H), 3.11-3.00 (m, 2H), 2.71-2.57 (m, 3H), 2.24 (s, 3H), 1.96 (d, J = 12.2 Hz, 2H), 1.47-1.41 (m, 1H), 1.04 (t, J = 7.1 Hz, 3H). |
| 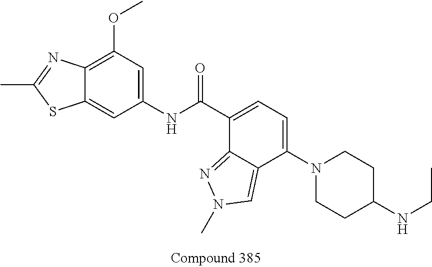 [SMILES: CCNC1CCN(c2ccc(C(=O)Nc3cc(OC)c4nc(C)sc4c3)c3nn(C)cc23)CC1] | 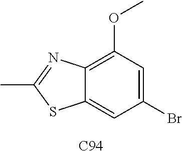 | Modification: Step 1 was ran at 90° C. for 3 h. The residue was purified by reversed- phase flash chromatography (Condition 4, Gradient 2) to afford 4-[4- (ethylamino)-1- piperidyl]-N-(4- methoxy-2-methyl- 1,3-benzothiazol-6- yl)-2-methyl-indazole- 7-carboxamide 2,2,2-trifluoroacetate (Compound 385, 50 mg, 84.5 μmol, 49% yield) as a solid. LCMS (ES, m/z): 479 [M + H]⁺. ¹H NMR (400 MHz, DMSO- d₆) δ 11.33 (s, 1H), 8.85 (s, 1H), 8.54 (s, 2H), 8.05-7.99 (m, 2H), 7.54 (d, J = 1.8 Hz, 1H), 6.56 (d, J = 8.2 Hz, 1H), 4.33 (s, 3H), 4.04 (d, J = 13.1 Hz, 2H), 4.00 (s, 3H), 3.35 (s, 1H), 3.09- 2.98 (m, 4H), 2.76 (s, 3H), 2.14 (d, J = 11.8 Hz, 2H), 1.76- 1.68 (m, 2H), 1.23 (t, J = 7.2 Hz, 3H). |
| 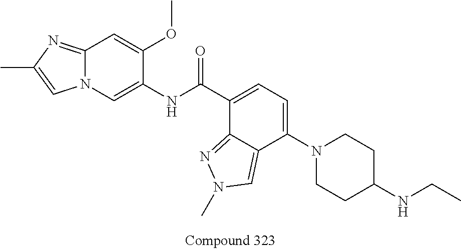 [SMILES: CCNC1CCN(c2ccc(C(=O)Nc3cn4cc(C)nc4cc3OC)c3nn(C)cc23)CC1] | 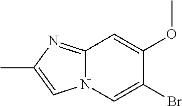 | Modification: Step 1 was ran at 90° C. The crude product was purified by Prep- HPLC (Condition 2, Gradient 6) to afford 4-[4-(ethylamino)- 1-piperidyl]-N-(7- methoxy-2-methyl- imidazo[1,2-a]- pyridin-6-yl)-2- methyl-indazole-7- carboxamide 2,2,2- trifluoroacetate (Compound 323, 21.2 mg, 45.9 μmol, 37% yield) as a solid. LCMS (ES, m/z): 462 [M + H]⁺. ¹H NMR (400 MHz, DMSO-d₆) δ 14.0- 13.92 (m, 1H), 11.79 (s, 1H), 9.93 (s, 1H), 8.88 (s, 1H), 8.83- 8.75 (m, 2H), 8.08- 8.02 (m, 2H), 7.36 (s, 1H), 6.57 (d, J = 8.2 Hz, 1H), 4.30 (s, 3H), 4.25 (s, 3H), 4.08 (d, J = 13.0 Hz, 2H), 3.07 (t, J = 11.9 Hz, 4H), 2.44 (d, J = 1.1 Hz, 3H), 2.17 (d, J = 11.7 Hz, 2H), 1.74 (d, J = 12.3 Hz, 2H), 1.25 (t, J = 7.2 Hz, 3H). |

An analogous method was followed using B90 as the starting material, in place of B9, to obtain the following compounds.

| | Starting | |
|---|---|---|
| Compound | Material | Characterization |
| 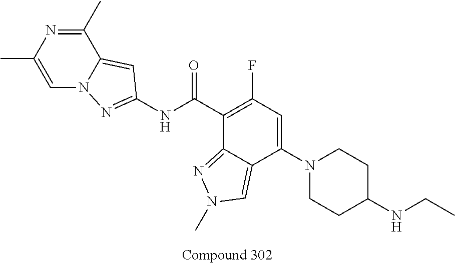 [SMILES: CCNC1CCN(c2cc(F)c(C(=O)Nc3cc4c(C)nc(C)cn4n3)c3nn(C)cc23)CC1] | 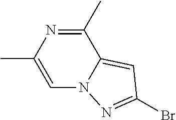 | Modification: Step 1 was ran for 3 h. Step 2 was ran for 1 h. The residue was purified by reversed- phase flash chromatography (Condition 3, Gradient 13) to afford N-(4,6- dimethylpyrazolo [1,5-a]pyrazin-2- yl)-4-[4- (ethylamino)-1- piperidyl]-6- fluoro-2-methyl- indazole-7- carboxamide (Compound 302, 0.027 g, 58.1 μmol, 41% yield) as a solid. LCMS (ES, m/z): 465 [M + H]⁺. ¹H NMR (300 MHz, DMSO-d₆) δ 11.71 (s, 1H), 8.81 (s, 1H), 8.41- 8.35 (m, 1H), 7.24 (d, J = 1.0 Hz, 1H), 6.24 (d, J = 16.1 Hz, 1H), 4.22 (s, 3H), 3.92 (d, J = 13.1 Hz, 2H), 3.13 (t, J = 11.6 Hz, 2H), 2.76-2.52 (m, 6H), 2.40 (d, J = 1.0 Hz, 3H), 1.95 (d, J = 12.7 Hz, 2H), 1.62 (s, 1H), 1.42 (t, J = 10.4 Hz, 2H), 1.03 (t, J = 7.1 Hz, 3H). |
| 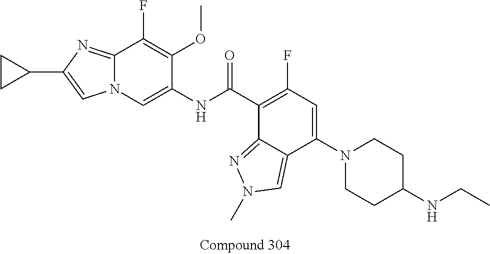 [SMILES: CCNC1CCN(c2cc(F)c(C(=O)Nc3cn4cc(C5CC5)nc4c(F)c3OC)c3nn(C)cc23)CC1] | 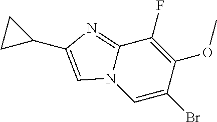 | Modification: Step 1 was ran at 90° C. The residue was purified by reversed- phase flash chromatography (Condition 3, Gradient 27) to afford N-(2- cyclopropyl- 8-fluoro-7- methoxyimidazo [1,2-a]pyridin-6- yl)-4-[4-(ethylamino)- piperidin-1- yl]-6-fluoro-2- methylindazole-7- carboxamide (Compound 304, 38 mg, 35% yield) as a solid. LCMS (ES, m/z): 524 [M + H]⁺. ¹H NMR (300 MHz, DMSO- d₆) δ 11.56 (s, 1H), 9.43 (s, 1H), 8.83 (s, 1H), 7.81 (d, J = 3.2 Hz, 1H), 6.24 (d, J = 16.2 Hz, 1H), 4.24 (s, 3H), 4.18 (d, J = 1.9 Hz, 3H), 3.94 (d, J = 13.0 Hz, 2H), 3.14 (t, J = 11.9 Hz, 2H), 2.77 (s, 1H), 2.64 (q, J = 7.1 Hz, 2H), 1.98 (t, J = 9.2 Hz, 3H), 1.48- 1.34 (m, 2H), 1.05 (t, J = 7.1 Hz, 3H), 0.86 (ddt, J = 23.3, 5.0, 2.4 Hz, 4H). |
| 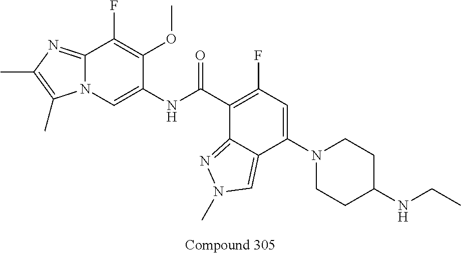 [SMILES: CCNC1CCN(c2cc(F)c(C(=O)Nc3cn4c(C)c(C)nc4c(F)c3OC)c3nn(C)cc23)CC1] | 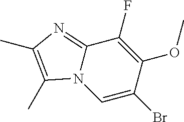 | Modification: Step 1 was ran at 90° C. for 5 h. The residue was purified by reversed- phase flash chromatography (Condition 3, Gradient 6) to afford 4-[4- (ethylamino) piperidin-1-yl]-6- fluoro-N-(8-fluoro- 7-methoxy- 2,3-dimethyl- imidazo[1,2- a]pyridin-6-yl)- 2-methylindazole- 7-carboxamide (Compound 305, 35 mg, 35% yield) as a solid. LCMS (ES, m/z): 512 [M + H]⁺. ¹H NMR (300 MHz, DMSO-d₆) δ 11.64 (s, 1H), 9.15 (s, 1H), 8.83 (s, 1H), 6.25 (d, J = 16.3 Hz, 1H), 4.28-4.15 (m, 6H), 3.93 (d, J = 12.8 Hz, 2H), 3.15 (t, J = 11.8 Hz, 2H), 2.73 (s, 1H), 2.61 (q, J = 7.1 Hz, 2H), 2.38 (s, 3H), 2.31 (s, 3H), 2.01-1.90 (m, 2H), 1.68 (s, 1H), 1.41 (d, J = 11.2 Hz, 2H), 1.04 (t, J = 7.1 Hz, 3H). |
| 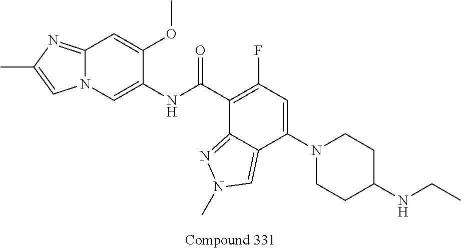 [SMILES: CCNC1CCN(c2cc(F)c(C(=O)Nc3cn4cc(C)nc4cc3OC)c3nn(C)cc23)CC1] | 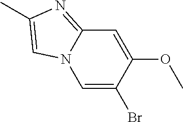 | Modification: Step 1 was ran for 2 h. The crude product was purified by Prep-HPLC (Condition 5, Gradient 14) to afford 4- [4-(ethylamino)-1- piperidyl]-6-fluoro- N-(7-methoxy- 2-methyl-imidazo [1,2-a]pyridin-6- yl)-2-methyl- indazole-7- carboxamide (Compound 331, 15.5 mg, 32.3 μmol, 21% yield) as a solid. LCMS (ES, m/z): 480 [M + H]⁺. ¹H NMR (400 MHz, DMSO-d₆) δ 11.60 (s, 1H), 9.53 (s, 1H), 8.80 (s, 1H), 7.54 (s, 1H), 6.95 (s, 1H), 6.22 (d, J = 16.2 Hz, 1H), 4.23 (s, 3H), 4.05 (s, 3H), 3.91 (d, J = 13.1 Hz, 2H), 3.12 (t, J = 11.3 Hz, 2H), 2.69 (dt, J = 9.7, 5.4 Hz, 1H), 2.60 (q, J = 7.1 Hz, 2H), 2.24 (s, 3H), 1.94 (d, J = 12.6 Hz, 2H), 1.65 (m, 1H), 1.47- 1.33 (m, 2H), 1.03 (t, J = 7.1 Hz, 3H). |
| 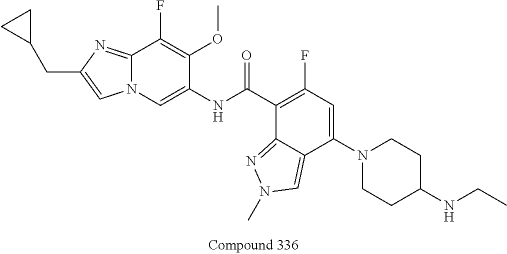 [SMILES: CCNC1CCN(c2cc(F)c(C(=O)Nc3cn4cc(CC5CC5)nc4c(F)c3OC)c3nn(C)cc23)CC1] | 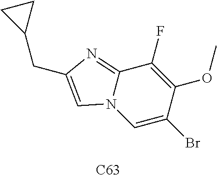 | Modification: Step 1 was ran at 90° C. for 16 h. The crude product was purified by Prep-HPLC (Condition 5, Gradient 19a) to afford N- [2-(cyclopropyl- methyl)-8-fluoro-7- methoxyimidazo- [1,2-a]pyridin-6- yl]-4-[4- (ethylamino)-1- piperidyl]-6- fluoro-2-methyl- indazole-7- carboxamide (Compound 336, 9 mg, 16.74 μmol, 27% yield) as a solid. LCMS (ES, m/z): 538 [M + H]⁺. ¹H NMR (300 MHz, DMSO-d₆) δ 11.57 (s, 1H), 9.47 (d, J = 1.2 Hz, 1H), 8.83 (s, 1H), 7.83 (d, J = 3.2 Hz, 1H), 6.23 (d, J = 16.3 Hz, 1H), 4.24 (s, 3H), 4.19 (d, J = 1.9 Hz, 3H), 3.93 (d, J = 12.9 Hz, 2H), 3.14 (t, J = 11.6 Hz, 2H), 2.77-2.67 (m, 1H), 2.59 (t, J = 6.8 Hz, 4H), 1.94 (d, J = 12.9 Hz, 2H), 1.40 (q, J = 9.9 Hz, 2H), 1.04 (q, J = 7.3 Hz, 4H), 0.55-0.41 (m, 2H), 0.26-0.12 (m, 2H). |
| 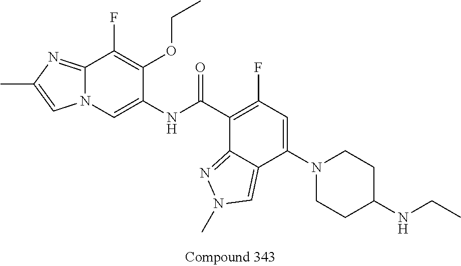 [SMILES: CCNC1CCN(c2cc(F)c(C(=O)Nc3cn4cc(C)nc4c(F)c3OCC)c3nn(C)cc23)CC1] | 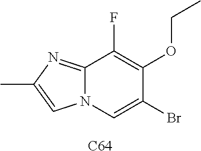 | Modification: Step 1 was ran at 90° C. The residue was purified by Prep- HPLC (Condition 1, Gradient 2) to afford N-(7-ethoxy- 8-fluoro-2- methylimidazo [1,2-a]pyridin-6- yl)-4-(4-(ethylamino) piperidin-1- yl)-6-fluoro- 2-methyl-2H- indazole-7- carboxamide hydrochloride (Compound 343, 0.04 g, 73 μmol, 37% yield) as a solid. LCMS (ES, m/z): 512 [M + H]⁺. ¹H NMR (300 MHz, DMSO-d₆) δ 11.54 (s, 1H), 9.82 (s, 1H), 9.19 (s, 2H), 8.93 (s, 1H), 8.10 (d, J = 2.2 Hz, 1H), 6.33 (d, J = 16.1 Hz, 1H), 4.69 (qd, J = 6.9, 2.3 Hz, 2H), 4.23 (s, 3H), 4.11 (d, J = 13.2 Hz, 2H), 3.11 (t, J = 12.5 Hz, 2H), 2.99 (d, J = 5.5 Hz, 1H), 2.54 (s, 2H), 2.48-2.41 (m, 3H), 2.18 (d, J = 12.2 Hz, 2H), 1.77 (q, J = 12.0, 11.5 Hz, 2H), 1.52 (t, J = 7.0 Hz, 3H), 1.26 (t, J = 7.2 Hz, 3H). |
| 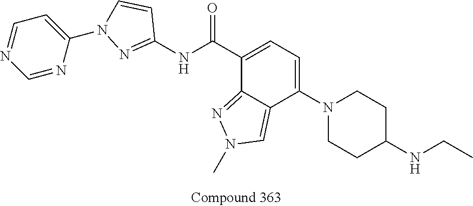 [SMILES: CCNC1CCN(c2ccc(C(=O)Nc3ccn(-c4ccncn4)n3)c3nn(C)cc23)CC1] | 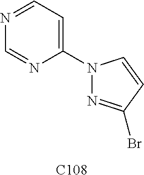 [SMILES: Brc1ccn(-c2ccncn2)n1 ; "C108"] | The residue was purified by reversed- phase flash chromatography (Condition 3, Gradient 11) to afford 4-(4- (ethylamino)- piperidin-1-yl)-2- methyl-N-(1- (pyrimidin-4-yl)-1H- pyrazol-3-yl)- 2H-indazole-7- carboxamide 2,2,2- trifluoroacetate (Compound 363, 40 mg, 90 μmol, 61% yield) as a solid. LCMS (ES, m/z): 446 [M + H]⁺. ¹H NMR (400 MHz, DMSO-d₆) δ 11.55 (s, 1H), 9.07 (d, J = 1.2 Hz, 1H), 8.92-8.86 (m, 2H), 8.70 (d, J = 2.8 Hz, 1H), 8.50 (s, 2H), 8.06 (d, J = 8.1 Hz, 1H), 7.85 (dd, J = 5.6, 1.3 Hz, 1H), 7.18 (d, J = 2.8 Hz, 1H), 6.58 (d, J = 8.2 Hz, 1H), 4.31 (s, 3H), 4.07 (d, J = 13.0 Hz, 2H), 3.38 (d, J = 11.6 Hz, 1H), 3.12- 3.01 (m, 4H), 2.14 (d, J = 12.0 Hz, 2H), 1.77-1.64 (m, 2H), 1.23 (t, J = 7.2 Hz, 3H). |
| 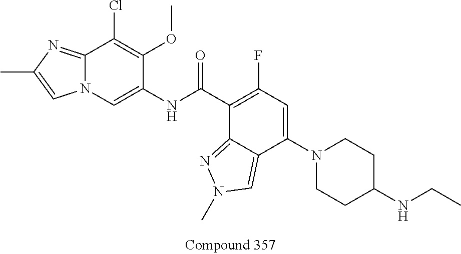 [SMILES: CCNC1CCN(c2cc(F)c(C(=O)Nc3cn4cc(C)nc4c(Cl)c3OC)c3nn(C)cc23)CC1] | 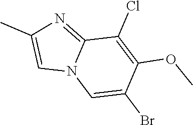 | Modification: Step 1 was ran for 16 h. The crude product was purified by Prep-HPLC (Condition 5, Gradient 15) to afford N-(8-chloro-7- methoxy-2- methylimidazo[1,2- a]pyridin-6-yl)-4- [4-(ethylamino)- 1-piperidyl]-6- fluoro-2-methyl- indazole-7- carboxamide (Compound 357, 40 mg, 77.8 μmol, 56% yield) as a solid. LCMS (ES, m/z): 514 [M + H]⁺. ¹H NMR (400 MHz, DMSO-d₆) δ 11.56 (s, 1H), 9.64 (s, 1H), 8.84 (s, 1H), 7.81 (s, 1H), 6.25 (d, J = 16.2 Hz, 1H), 4.25 (s, 3H), 4.08 (s, 3H), 3.93 (d, J = 13.3 Hz, 2H), 3.15 (t, J = 12.0 Hz, 2H), 2.69 (d, J = 12.0 Hz, 1H), 2.60 (q, J = 7.1 Hz, 2H), 2.32 (s, 3H), 1.95 (d, J = 12.9 Hz, 2H), 1.41 (q, J = 10.4, 9.9 Hz, 2H), 1.04 (t, J = 7.1 Hz, 3H). |

An analogous method was followed, using B98 in place of B9, to obtain the following compounds.

| | Starting | |
|---|---|---|
| Compound | Material | Characterization |
| 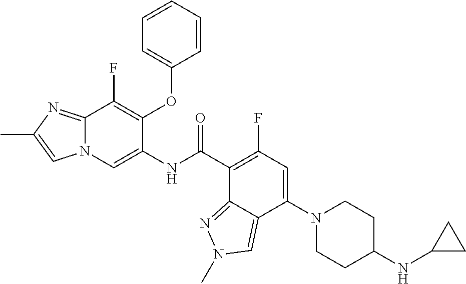 [SMILES: Cc1cn2cc(NC(=O)c3c(F)cc(N4CCC(NC5CC5)CC4)c4cn(C)nc34)c(Oc3ccccc3)c(F)c2n1] Compound 377 | 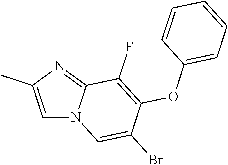 C79 | The residue was purified by reversed- phase flash chromatography (Condition 3, Gradient 5) to afford 4-[4- (cyclopropylamino)- 1-piperidyl]- 6-fluoro-N-(8- fluoro-2-methyl-7- phenoxyimidazo [1,2-a]pyridin-6- yl)-2-methyl- indazole-7- carboxamide (Compound 377, 37 mg, 64.7 μmol, 46% yield) as a solid. LCMS (ES, m/z): 572 [M + H]⁺. ¹H NMR (400 MHz, DMSO-d₆) δ 11.48 (s, 1H), 9.63 (d, J = 1.2 Hz, 1H), 8.69 (s, 1H), 7.91 (d, J = 3.0 Hz, 1H), 7.44-7.34 (m, 2H), 7.22-7.09 (m, 3H), 6.20 (d, J = 16.3 Hz, 1H), 3.87 (dt, J = 13.2, 4.1 Hz, 2H), 3.77 (s, 3H), 3.10 (ddd, J = 13.4, 11.0, 2.8 Hz, 2H), 2.78 (tt, J = 9.4, 4.0 Hz, 1H), 2.34 (s, 4H), 2.09 (tt, J = 6.7, 3.6 Hz, 1H), 1.99-1.91 (m, 2H), 1.54-1.29 (m, 2H), 0.38 (dt, J = 6.2, 3.1 Hz, 2H), 0.25- 0.17 (m, 2H). |
| 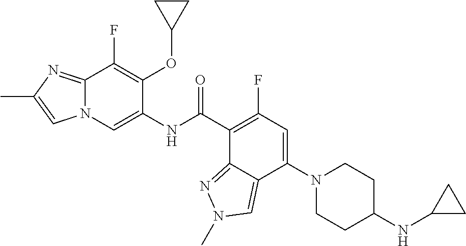 [SMILES: Cc1cn2cc(NC(=O)c3c(F)cc(N4CCC(NC5CC5)CC4)c4cn(C)nc34)c(OC3CC3)c(F)c2n1] Compound 382 | 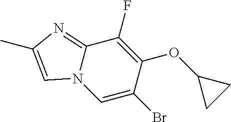 C80 | Modification: Step 1 was ran at 90° C. for 16 h. The residue was purified by reversed- phase flash chromatography (Condition 3, Gradient 5) to afford N-[7- (cyclopropoxy)- 8-fluoro-2- methyl-imidazo [1,2-a]-pyridin-6- yl]-4-[4- (cyclopropylamino)-1- piperidyl]-6- fluoro-2-methyl- indazole-7- carboxamide (Compound 382, 26 mg, 48.5 μmol, 36% yield) as a solid. LCMS (ES, m/z): 535 [M + H]⁺. ¹H NMR (300 MHz, DMSO-d₆) δ 11.21 (s, 1H), 9.44 (d, J = 1.2 Hz, 1H), 8.82 (s, 1H), 7.75 (dd, J = 3.2, 1.1 Hz, 1H), 6.21 (d, J = 16.3 Hz, 1H), 4.45 (tq, J = 5.8, 2.8 Hz, 1H), 4.22 (s, 3H), 3.89 (dd, J = 10.4, 6.9 Hz, 2H), 3.19-3.04 (m, 2H), 2.84- 2.72 (m, 1H), 2.30 (s, 4H), 2.09 (tt, J = 6.8, 3.6 Hz, 1H), 2.02-1.91 (m, 2H), 1.51-1.32 (m, 2H), 0.94 (pt, J = 3.2, 1.9 Hz, 2H), 0.86-0.66 (m, 2H), 0.37 (dt, J = 6.2, 3.0 Hz, 2H), 0.26-0.15 (m, 2H). |
| 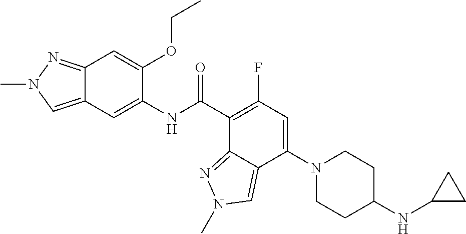 [SMILES: CCOc1cc2nn(C)cc2cc1NC(=O)c1c(F)cc(N2CCC(NC3CC3)CC2)c2cn(C)nc12] Compound 415 | 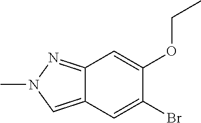 C166 | Modification: Step 1 was ran for 2 h at 90° C. The crude product was purified by Prep-HPLC (Condition 5, Gradient 19) to afford 4-[4- (cyclopropyl-amino)- 1-piperidyl]- N-(6-ethoxy-2- methylindazol-5- yl)-6-fluoro-2- methylindazole-7- carboxamide (Compound 415, 44 mg, 87 μmol, 61% yield) as a solid. LCMS (ES, m/z): 506 [M + H]⁺. ¹H NMR (400 MHz, DMSO-d₆) δ 11.12 (s, 1H), 8.78 (d, J = 6.5 Hz, 2H), 8.16 (s, 1H), 7.05 (s, 1H), 6.22 (d, J = 16.1 Hz, 1H), 4.26 (t, J = 7.0 Hz, 2H), 4.22 (s, 3H), 4.08 (s, 3H), 3.89 (d, J = 13.0 Hz, 2H), 3.09 (t, J = 11.7 Hz, 2H), 2.84- 2.73 (m, 1H), 2.28 (s, 1H), 2.12 (dt, J = 6.6, 3.2 Hz, 1H), 1.99 (d, J = 12.8 Hz, 2H), 1.52 (t, J = 7.0 Hz, 3H), 1.45 (q, J = 9.8 Hz, 2H), 0.40 (dt, J = 6.2, 3.1 Hz, 2H), 0.30-0.18 (m, 2H). |
| 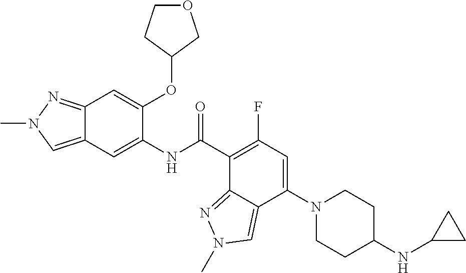 [SMILES: Cn1cc2cc(NC(=O)c3c(F)cc(N4CCC(NC5CC5)CC4)c4cn(C)nc34)c(OC3CCOC3)cc2n1] Compound 467 | 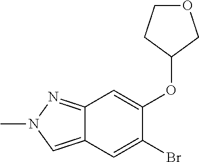 D3 | The residue was reversed- phase flash chromatography (Condition 3, Gradient 22) to afford 4-[4- (cyclopropyl-amino)- 1-piperidyl]- 6-fluoro-2-methyl- N-(2-methyl-6- tetrahydrofuran-3- yloxy-indazol- 5-yl)indazole-7- carboxamide (Compound 467, 8 mg, 14.6 μmol). LCMS (ES, m/z): 548 [M + H]⁺. ¹H NMR (400 MHz, DMSO-d₆): δ 10.88 (s, 1H), 8.78 (d, J = 5.0 Hz, 2H), 8.19 (s, 1H), 7.05 (s, 1H), 6.22 (d, J = 16.1 Hz, 1H), 5.23 (s, 1H), 4.23 (s, 3H), 4.09 (s, 3H), 4.04 (d, J = 3.4 Hz, 2H), 3.90 (dd, J = 14.4, 7.0 Hz, 3H), 3.79 (td, J = 8.2, 5.0 Hz, 1H), 3.09 (t, J = 11.8 Hz, 2H), 2.79 (s, 1H), 2.37 (dd, J = 13.8, 6.6 Hz, 2H), 2.15-2.10 (m, 2H), 1.99 (d, J = 13.0 Hz, 2H), 1.52-1.42 (m, 2H), 0.40 (d, J = 4.7 Hz, 2H), 0.24 (d, J = 3.6 Hz, 2H). |

### Example 95: Synthesis of C43

### Synthesis of C40

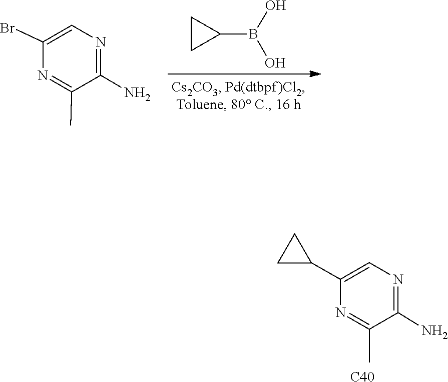

To a stirred solution of 5-bromo-3-methyl-pyrazin-2-amine (500 mg, 2.66 mmol, 1 eq) and cyclopropylboronic acid (685 mg, 7.98 mmol, 3 eq) in toluene (5 mL) were added Cs₂CO₃ (2.6 g, 7.98 mmol, 3 eq) and Pd(dtbpf)Cl₂ (173 mg, 265.9 μmol, 0.1 eq) at room temperature under N₂. The resulting mixture was stirred for 16 h at 80° C. under N₂. The resulting mixture was concentrated under reduced pressure and purified by silica gel column chromatography (17% THF in PE) to afford 5-cyclopropyl-3-methyl-pyrazin-2-amine (C40, 350 mg, 84% yield) as a solid. LCMS (ES, m/z): 150 [M+H]⁺.

### Synthesis of C41

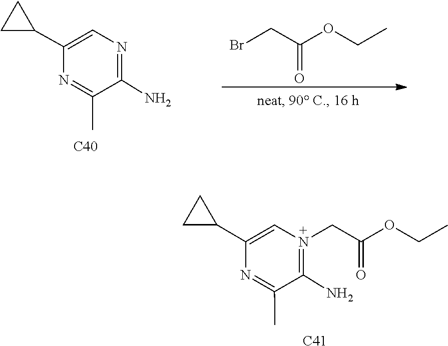

To a stirred solution of 5-cyclopropyl-3-methyl-pyrazin-2-amine (300 mg, 2.01 mmol, 1 eq) in ethyl 2-bromoacetate (2.01 g, 12.06 mmol, 6 eq), and the reaction was stirred for 16 h at 90° C. The reaction mixture was cooled to room temperature. The precipitated solids were collected by filtration and washed with EtOAc (3×50 mL). The filter cake is ethyl 2-(2-amino-5-cyclopropyl-3-methyl-pyrazin-1-ium-1-yl)acetate (300 mg, 63% yield) as a solid. LCMS (ES, m/z): 236 [M+H]⁺.

### Synthesis of C42

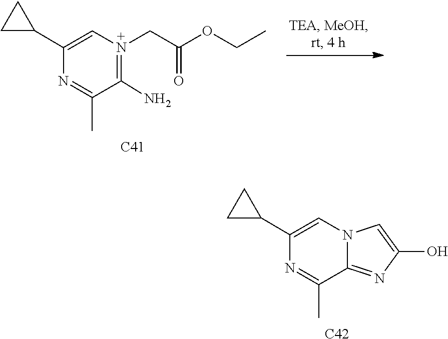

To a stirred solution of ethyl 2-(2-amino-5-cyclopropyl-3-methyl-pyrazin-1-ium-1-yl)-acetate (270 mg, 1.14 mmol, 1 eq) in MeOH (10 mL) was added TEA (346 mg, 3.43 mmol, 3 eq), and the reaction was stirred for 4 h at rt. The precipitated solids were collected by filtration, washed with EtOAc (3×50 mL), and dried to afford 6-cyclopropyl-8-methylimidazo[1,2-a]-pyrazin-2-ol (205 mg, 95% yield) as a solid. LCMS (ES, m/z): 190 [M+H]⁺.

### Synthesis of C43

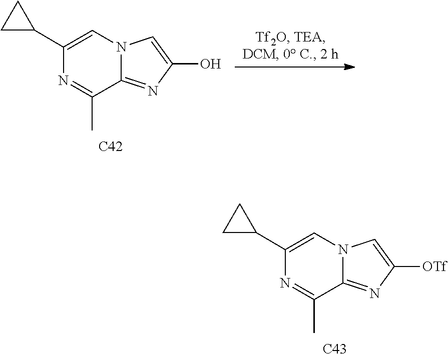 [SMILES: Cc1nc(C2CC2)cn2cc(O)nc12 ; "C42" ; Cc1nc(C2CC2)cn2cc(OS(=O)(=O)C(F)(F)F)nc12 ; "C43"]

To a stirred solution of 6-cyclopropyl-8-methyl-imidazo[1,2-a]pyrazin-2-ol (200 mg, 1.06 mmol, 1 eq) in DCM (4 mL) were added TEA (213 mg, 2.11 mmol, 2 eq) and Tf₂O (447 mg, 1.59 mmol, 1.5 eq), and the reaction was stirred for 2 h at 0° C. The resulting mixture was diluted with water (20 mL) and extracted with DCM (3×50 mL). The combined organic layers were washed with brine (50 mL), dried over anhydrous Na₂SO₄, and filtered. The filtrate was concentrated under reduced pressure and purified by silica gel column chromatography (50% THF in PE) to afford (6-cyclopropyl-8-methyl-imidazo[1,2-a]pyrazin-2-yl)trifluoromethanesulfonate (300 mg, 88% yield) as a solid. LCMS (ES, m/z): 322 [M+H]⁺.

An analogous method was followed to obtain the following intermediate.

| | Starting | |
|---|---|---|
| Compound | Material | Characterization |
| 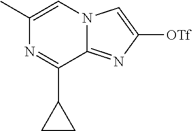 [SMILES: Cc1cn2cc(OS(=O)(=O)C(F)(F)F)nc2c(C2CC2)n1] C44 | 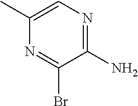 | The residue was purified by silica gel column chromatography (50% EtOAc in PE) to afford (8- cyclopropyl-6-methyl-imidazo[1,2-a]pyrazin-2-yl) trifluoromethanesulfonate (C44, 150 mg, 466.9 μmol, 80% yield) as an oil. LCMS (ES, m/z): 322 [M + H]⁺. |

### Example 96: Synthesis of Compound 312

### Synthesis of B83

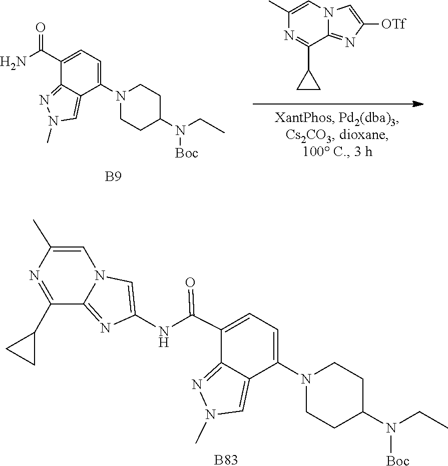

Into an 8 mL vial were added tert-butyl N-[1-(7-carbamoyl-2-methyl-indazol-4-yl)-4-piperidyl]-N-ethyl-carbamate (100 mg, 249.1 μmol), dioxane (2 mL), 8-cyclopropyl-6-methylimidazo[1,2-a]pyrazin-2-yl trifluoromethanesulfonate (120 mg, 373.6 μmol), Cs₂CO₃ (243 mg, 747.2 μmol), Xantphos (28 mg, 49.81 μmol) and Pd₂(dba)₃ (22 mg, 24.91 μmol), and the reaction was stirred for 3 h at 100° C. under N₂. The resulting mixture was concentrated under reduced pressure and purified by silica gel column chromatography (50% THF in PE) to afford tert-butyl N-[1-[7-[(8-cyclopropyl-6-methyl-imidazo[1,2-a]pyrazin-2-yl)carbamoyl]-2-methyl-indazol-4-yl]-4-piperidyl]-N-ethyl-carbamate (B83, 35 mg, 61.11 μmol, 25% yield) as a solid. LCMS (ES, m/z): 573 [M+H]⁺.

### Synthesis of Compound 312

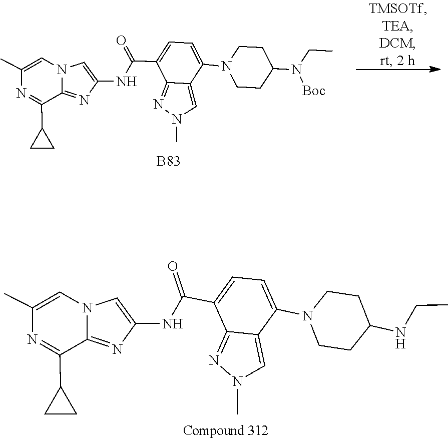

Into an 8 mL vial were added tert-butyl N-[1-[7-[(8-cyclopropyl-6-methyl-imidazo[1,2-a]pyrazin-2-yl)carbamoyl]-2-methyl-indazol-4-yl]-4-piperidyl]-N-ethyl-carbamate (30 mg, 52.38 μmol), DCM (0.6 mL), TMSOTf (23 mg, 104.8 μmol) and TEA (15 mg, 157.2 μmol), and the reaction was stirred for 2 h at room temperature. The resulting mixture was concentrated under reduced pressure and purified by reversed-phase flash chromatography (Condition 1, Gradient 6) to afford N-(8-cyclopropyl-6-methyl-imidazo[1,2-a]pyrazin-2-yl)-4-[4-(ethylamino)-1-piperidyl]-2-methyl-indazole-7-carboxamide (Compound 312, 4.5 mg, 9.52 μmol, 18% yield) as a solid. LCMS (ES, m/z): 473 [M+H]⁺. ¹H NMR (400 MHz, Methanol-d₄) δ 8.49 (d, J=3.6 Hz, 1H), 8.26 (d, J=3.2 Hz, 1H), 8.09 (dd, J=8.1, 2.6 Hz, 1H), 8.02 (s, 1H), 6.53 (dd, J=8.4, 3.3 Hz, 1H), 4.35 (s, 3H), 4.03 (d, J=12.7 Hz, 2H), 3.04 (t, J=12.4 Hz, 2H), 2.82-2.72 (m, 4H), 2.38 (s, 3H), 2.14-2.06 (m, 2H), 1.61-1.55 (m, 2H), 1.27 (p, J=4.0 Hz, 2H), 1.22-1.10 (m, 5H).

An analogous method was followed to obtain the following compounds.

| Compound | Starting Material | Characterization |
|---|---|---|
| 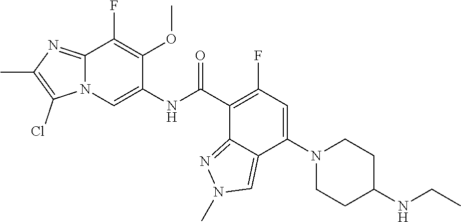 [SMILES: CCNC1CCN(c2cc(F)c(C(=O)Nc3cn4c(Cl)c(C)nc4c(F)c3OC)c3nn(C)cc23)CC1] Compound 308 | 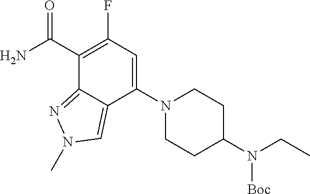 B90 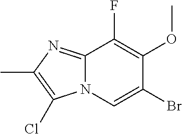 | Modifications: Step 1 was ran for 2 h at 80° C. Step 2 was ran using DIEA at 50° C. for 16 h. The crude mixture was purified by reversed- phase flash chromatography (Condition 3, Gradient 5) to afford N-(3-chloro-8-fluoro-7-methoxy-2-methyl-imidazo [1,2-a]pyridin-6-yl)-4-[4-(ethylamino)-1-piperidyl]-6-fluoro- 2-methyl-indazole-7-carboxamide (Compound 308, 60 mg, 112.8 μmol, 42% yield) as a solid. LCMS (ES, m/z): 532 [M + H]⁺. ¹H NMR (300 MHz, DMSO-d₆) δ 11.71 (s, 1H), 9.36-9.23 (m, 1H), 8.79 (s, 1H), 6.21 (d, J = 16.3 Hz, 1H), 4.23 (d, J = 1.9 Hz, 6H), 3.92 (d, J = 13.2 Hz, 2H), 3.14 (t, J = 11.8 Hz, 2H), 2.71 (dd, J = 9.3, 4.8 Hz, 1H), 2.61 (q, J = 7.1 Hz, 2H), 2.32 (s, 3H), 2.00-1.89 (m, 2H), 1.41 (q, J = 10.4 Hz, 2H), 1.04 (t, J = 7.1 Hz, 3H). |
| 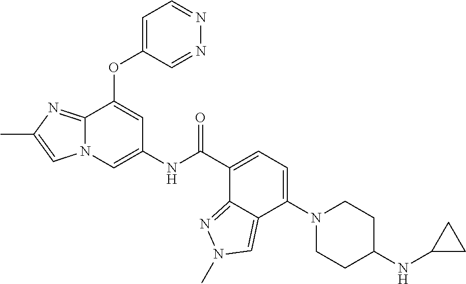 [SMILES: Cc1cn2cc(NC(=O)c3ccc(N4CCC(NC5CC5)CC4)c4cn(C)nc34)cc(Oc3ccnnc3)c2n1] Compound 322 | 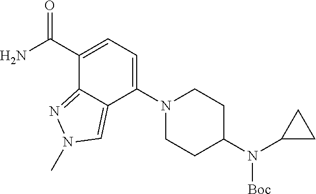 B102 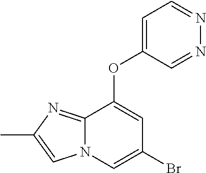 C82 | The residue was purified by Prep-HPLC (Condition 5, Gradient 10a) to afford 4-[4-(cyclopropyl-amino)-1-piperidyl]-2-methyl-N-(2- methyl-8-pyridazin-4-yloxy-imidazo[1,2-a]-pyridin-6-yl)- indazole-7-carboxamide (Compound 322, 18.9 mg, 35.2 μmol, 37% yield) as a solid. LCMS (ES, m/z): 538 [M + H]⁺. ¹H NMR (400 MHz, DMSO-d₆) δ 11.12 (s, 1H), 9.40 (d, J = 1.8 Hz, 1H), 9.26-9.20 (m, 1H), 9.05 (d, J = 6.1 Hz, 1H), 8.77 (s, 1H), 7.98 (d, J = 8.0 Hz, 1H), 7.92 (s, 1H), 7.45 (d, J = 1.8 Hz, 1H), 7.18 (dd, J = 6.1, 3.1 Hz, 1H), 6.49 (d, J = 8.3 Hz, 1H), 4.26 (s, 3H), 3.89 (d, J = 12.6 Hz, 2H), 3.07 (t, J = 11.9 Hz, 2H), 2.78 (s, 1H), 2.26 (s, 3H), 2.13 (dd, J = 6.8, 3.5 Hz, 1H), 2.00 (d, J = 12.5 Hz, 2H), 1.47 (q, J = 9.9 Hz, 2H), 0.40 (dt, J = 6.2, 3.0 Hz, 2H), 0.27-0.19 (m,2H). |

### Example 97: Synthesis of C45

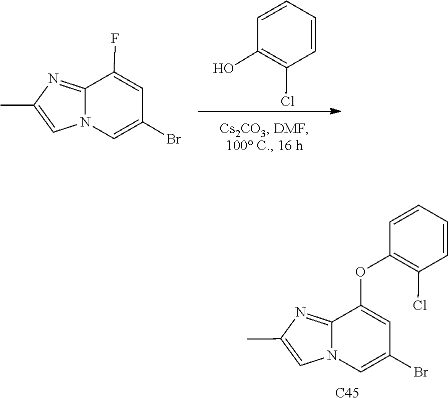 [SMILES: Cc1cn2cc(Br)cc(F)c2n1 ; Oc1ccccc1Cl ; Cc1cn2cc(Br)cc(Oc3ccccc3Cl)c2n1 ; "C45"]

To a stirred mixture of 6-bromo-8-fluoro-2-methyl-imidazo[1,2-a]pyridine (120 mg, 523.9 μmol) and 2-chlorophenol (101 mg, 785.9 μmol) in DMF (10 mL) were added Cs₂CO₃ (427 mg, 1.31 mmol) at room temperature under N₂. The resulting mixture was stirred for 16 h at 100° C. The mixture was diluted with water (40 mL) and extracted with EtOAc (2×40 mL). The combined organic layer was washed with water (80 mL) and brine (80 mL), dried over anhydrous Na₂SO₄, and filtered. The filtrate was concentrated under reduced pressure and purified by silica gel column chromatography (75% EtOAc in PE) to afford 6-bromo-8-(2-chlorophenoxy)-2-methyl-imidazo[1,2-a]pyridine (C45, 100 mg, 296.2 μmol, 57% yield) as a solid. LCMS (ES, m/z): 337 [M+H]⁺.

An analogous method was followed to obtain the following compound.

| | Starting | |
|---|---|---|
| Compound | Material | Characterization |
| 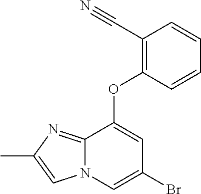 [SMILES: Cc1cn2cc(Br)cc(Oc3ccccc3C#N)c2n1] C46 | 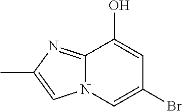 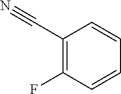 | The residue was purified by silica gel column chromatography (83% EtOAc in PE) to afford 2-(6- bromo-2-methyl-imidazo[1,2-a]pyridin-8-yl)- oxybenzonitrile (C46, 0.15 g, 457 μmol, 35% yield) as a solid. LCMS (ES, m/z): 328 [M + H]⁺. |
| 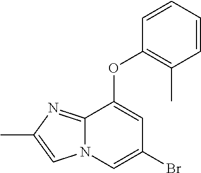 [SMILES: Cc1cn2cc(Br)cc(Oc3ccccc3C)c2n1] C47 | 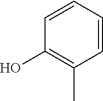 | The residue was purified by silica gel column chromatography (40% EtOAc in PE) to afford 6- bromo-2-methyl-8-(2-methylphenoxy)-imidazo[1,2- a]pyridine (C47, 410 mg, 1.3 mmol, 99% yield) as a solid. LCMS (ES, m/z): 317 [M + H]⁺. |
| 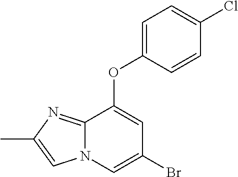 [SMILES: Cc1cn2cc(Br)cc(Oc3ccc(Cl)cc3)c2n1] C48 | 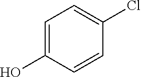 | The residue was purified by silica gel column chromatography (25% EtOAc in PE) to afford 6- bromo-8-(4-chlorophenoxy)-2-methyl-imidazo[1,2- a]pyridine (C48, 220 mg, 651.7 μmol, 75% yield) as a solid. LCMS (ES, m/z): 337 [M + H]⁺. |
| 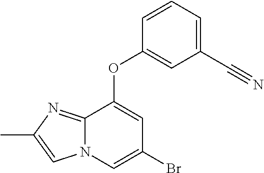 [SMILES: Cc1cn2cc(Br)cc(Oc3cccc(C#N)c3)c2n1] C49 | 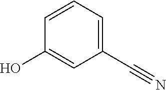 | The residue was purified by silica gel column chromatography (25% EtOAc in PE) to afford 3-(6- bromo-2-methyl-imidazo[1,2-a]pyridin-8-yl)- oxybenzonitrile (210 mg, 639.9 μmol, 73% yield) as a solid. LCMS (ES, m/z): 328 [M + H]⁺. |
| 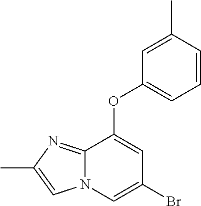 [SMILES: Cc1cccc(Oc2cc(Br)cn3cc(C)nc23)c1] C50 | 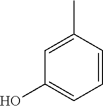 | The residue was purified by silica gel column chromatography (40% EtOAc in PE) to afford 6- bromo-2-methyl-8-(3-methylphenoxy)-imidazo[1,2- a]pyridine (C50, 275 mg, 867 μmol, 99% yield) as a solid. LCMS (ES, m/z): 317 [M + H]⁺. |
| 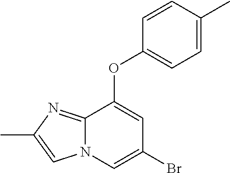 C51 | 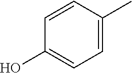 | The residue was purified by silica gel column chromatography (40% EtOAc in PE) to afford 6- bromo-2-methyl-8-(4-methylphenoxy)-imidazo[1,2- a]pyridine (C51, 260 mg, 819.7 μmol, 94% yield) as a solid. LCMS (ES, m/z): 317 [M + H]⁺. |
| 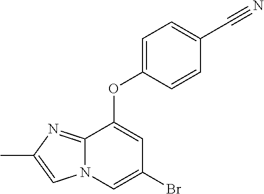 [SMILES: Cc1cn2cc(Br)cc(Oc3ccc(C#N)cc3)c2n1] C52 | 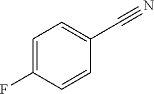 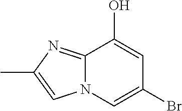 | The residue was purified by silica gel column chromatography (33% EtOAc in PE) to afford 4-(6- bromo-2-methyl-imidazo[1,2-a]pyridin-8-yl)- oxybenzonitrile (130 mg, 396.1 μmol, 45% yield) as a solid. LCMS (ES, m/z): 328 [M + H]⁺. |
| 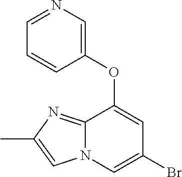 [SMILES: Cc1cn2cc(Br)cc(Oc3cccnc3)c2n1] C53 | 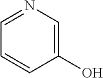 | The residue was purified by silica gel column chromatography (67% EtOAc in PE) to afford 6- bromo-2-methyl-8-(3-pyridyloxy)-imidazo[1,2-a]- pyridine (220 mg, 723.4 μmol, 83% yield) as a solid. LCMS (ES, m/z): 304 [M + H]⁺. |
| 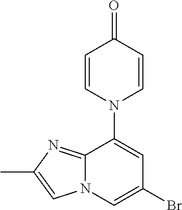 [SMILES: Cc1cn2cc(Br)cc(-n3ccc(=O)cc3)c2n1] C54 | 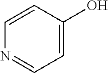 | The residue was purified by silica gel column chromatography (75% EtOAc in PE) to afford 1-(6- bromo-2-methyl-imidazo[1,2-a]pyridin-8-yl)- pyridin-4-one (C54, 0.26 g, 854.9 μmol, 65% yield) as a solid. LCMS (ES, m/z): 304 [M + H]⁺. |
| 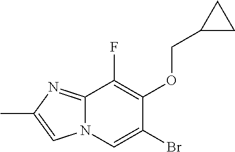 [SMILES: Cc1cn2cc(Br)c(OCC3CC3)c(F)c2n1] C60 | 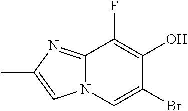 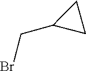 | Modification: The reaction was ran in MeCN at 110° C. for 2 h. To a stirred solution of 6-bromo-8-fluoro-2-methyl- imidazo[1,2-a]pyridin-7-ol (280 mg, 1.14 mmol) and bromomethylcyclopropane (309 mg, 2.29 mmol) in MeCN (5 mL) was added Cs₂CO₃ (1.12 g, 3.43 mmol) at room temperature under N2. The resulting mixture was stirred for 2 h at 110° C. under N₂. The mixture was allowed to cool down to room temperature. The reaction was quenched by the addition of water (50 mL) at room temperature. The resulting mixture was extracted with EtOAc (3 × 20 mL). The combined organic layers were washed with brine (30 mL), dried over anhydrous Na₂SO₄, and filtered. The filtrate was concentrated under reduced pressure and purified by silica gel column chromatography (33% THF in PE) to afford 6-bromo-7-(cyclopropylmethoxy)-8-fluoro-2- methyl-imidazo[1,2-a]pyridine (C60, 200 mg, 668.6 μmol, 59% yield) as a solid. LCMS (ES, m/z): 299 [M + H]. |
| 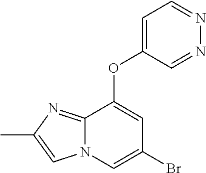 [SMILES: Cc1cn2cc(Br)cc(Oc3ccnnc3)c2n1] C82 | 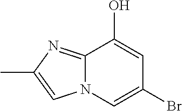 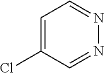 | The residue was purified by silica gel column chromatography (5% MeOH in DCM) to afford 6- bromo-2-methyl-8-pyridazin-4-yl-oxyimidazo-[1,2- a]pyridine (C82, 300 mg, 983.2 μmol, 74% yield) as a solid. LCMS (ES, m/z): 305 [M + H]⁺. |
| 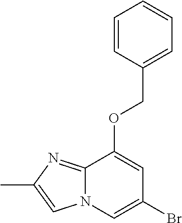 [SMILES: Cc1cn2cc(Br)cc(OCc3ccccc3)c2n1] C83 | 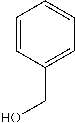 | The residue was purified by silica gel column chromatography (25% EtOAc in PE) to afford 8- benzyloxy-6-bromo-2-methyl-imidazo[1,2-a]- pyridine (C83, 210 mg, 662.1 μmol, 76% yield) as a solid. LCMS (ES, m/z): 317 [M + H]⁺. |
| 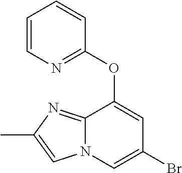 [SMILES: Cc1cn2cc(Br)cc(Oc3ccccn3)c2n1] C84 | 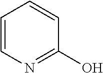 | The residue was purified by silica gel column chromatography (67% EtOAc in PE) to afford 6- bromo-2-methyl-8-(2-pyridyloxy)-imidazo[1,2-a]- pyridine (C84, 150 mg, 493.2 μmol, 23% yield) as a solid. LCMS (ES, m/z): 304 [M + H]⁺. |
| 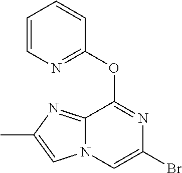 [SMILES: Cc1cn2cc(Br)nc(Oc3ccccn3)c2n1] C118 | 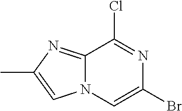 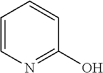 | Modification: The reaction was ran using K₂CO₃ in DMF, at 50° C. for 3 h. The residue was purified by silica gel column chromatography (67% EtOAc in PE) to afford 6- bromo-2-methyl-8-(2-pyridyloxy)-imidazo[1,2-a]- pyrazine (C118, 210 mg, 688.2 μmol, 34% yield) as a solid. LCMS (ES, m/z): 304 [M + H]⁺. |

### Example 98: Synthesis of Compound 316

### Synthesis of B84

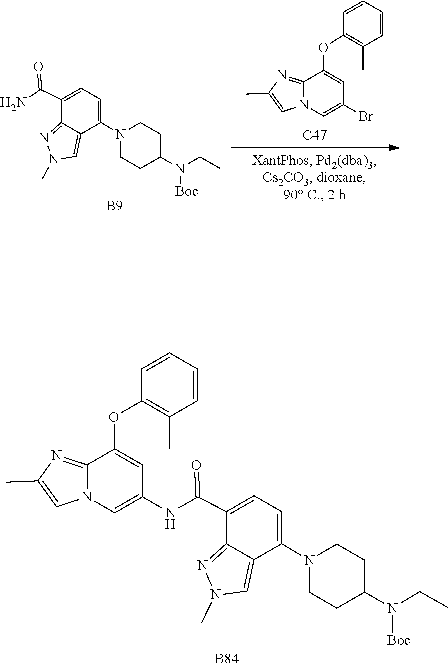

To a stirred mixture of tert-butyl N-[1-(7-carbamoyl-2-methyl-indazol-4-yl)-4-piperidyl]-N-ethyl-carbamate (100 mg, 249.1 μmol) in dioxane (1 mL) were added 6-bromo-2-methyl-8-(2-methylphenoxy)imidazo[1,2-a]pyridine (95 mg, 298.9 μmol), Cs₂CO₃ (162 mg, 498.1 μmol), Xantphos (29 mg, 49.81 μmol) and Pd₂(dba)₃ (23 mg, 24.91 μmol), and the reaction was stirred for 2 h at 90° C. under N₂. The resulting mixture was diluted with H₂O (15 mL) and extracted with DCM (2×15 mL). The combined organic layers were washed with water (2×20 mL), dried over anhydrous Na₂SO₄, and filtered. The filtrate was concentrated under reduced pressure and purified by silica gel column chromatography (60% EtOAc in DCM) to afford tert-butyl N-ethyl-N-[1-[2-methyl-7-[[2-methyl-8-(2-methylphenoxy)imidazo[1,2-a]pyridin-6-yl]-carbamoyl]indazol-4-yl]-4-piperidyl]carbamate (B84, 120 mg, 188.2 μmol, 76% yield) as a solid. LCMS (ES, m/z): 638 [M+H]⁺.

### Synthesis of Compound 316

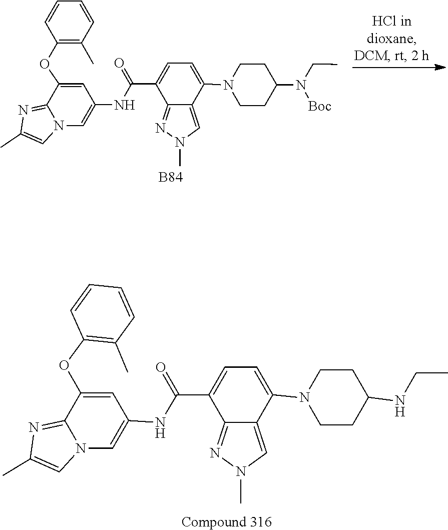

To a solution of tert-butyl N-ethyl-N-[1-[2-methyl-7-[[2-methyl-8-(2-methylphenoxy)-imidazo[1,2-a]pyridin-6-yl]carbamoyl]indazol-4-yl]-4-piperidyl]carbamate (110 mg, 172.5 μmol) in DCM (1.5 mL) was added HCl (4.0 M in 1,4-dioxane, 400 μL), and the reaction was stirred for 2 h at rt. The resulting mixture was concentrated under reduced pressure and purified by Prep-HPLC (Condition 14, Gradient 2) to afford 4-[4-(ethylamino)-1-piperidyl]-2-methyl-N-[2-methyl-8-(2-methylphenoxy)imidazo[1,2-a]pyridin-6-yl]indazole-7-carboxamide (Compound 316, 40 mg, 74.4 μmol, 43% yield) as a solid. LCMS (ES, m/z): 538 [M+H]⁺. ¹H NMR (400 MHz, DMSO-d₆) δ 10.89 (s, 1H), 9.02 (d, J=1.7 Hz, 1H), 8.73 (s, 1H), 7.92 (d, J=8.1 Hz, 1H), 7.83 (s, 1H), 7.43-7.36 (m, 1H), 7.26 (td, J=7.8, 1.8 Hz, 1H), 7.17 (td, J=7.4, 1.3 Hz, 1H), 7.00 (dd, J=7.9, 1.3 Hz, 1H), 6.57 (d, J=1.7 Hz, 1H), 6.46 (d, J=8.2 Hz, 1H), 4.16 (s, 3H), 3.90-3.82 (m, 2H), 3.09-2.98 (m, 2H), 2.68 (s, 1H), 2.61 (q, J=7.1 Hz, 2H), 2.33 (s, 3H), 2.28 (s, 3H), 1.99-1.91 (m, 2H), 1.43 (d, J=11.2 Hz, 2H), 1.04 (t, J=7.1 Hz, 3H).

An analogous method was followed to obtain the following compounds.

| Compounds | Starting Material | Characterization |
|---|---|---|
| 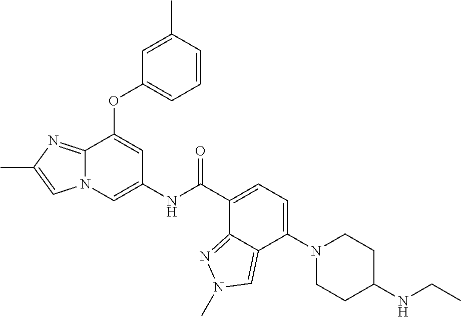 [SMILES: CCNC1CCN(c2ccc(C(=O)Nc3cc(Oc4cccc(C)c4)c4nc(C)cn4c3)c3nn(C)cc23)CC1] Compound 327 | 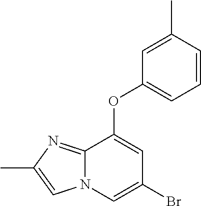 C50 | The crude product was purified by Prep-HPLC (Condition 14, Gradient 3) to afford 4-[4-(ethylamino)-1- piperidyl]-2-methyl-N-[2- methyl-8-(3- methylphenoxy)imidazo[1,2- a]pyridin-6-yl]indazole-7- carboxamide (Compound 327, 25 mg, 46.5 μmol, 27% yield) as a solid. LCMS (ES, m/z): 538 [M + H]⁺. ¹H NMR (400 MHz, DMSO-d₆) δ 10.96 (s, 1H), 9.14 (d, J = 1.7 Hz, 1H), 8.75 (s, 1H), 7.94 (d, J = 8.1 Hz, 1H), 7.84 (d, J = 1.1 Hz, 1H), 7.30 (t, J = 7.9 Hz, 1H), 7.01 (d, J = 7.5 Hz, 1H), 6.95 (d, J = 2.4 Hz, 1H), 6.90 (dd, J = 8.1, 2.6 Hz, 1H), 6.87 (d, J = 1.8 Hz, 1H), 6.47 (d, J = 8.3 Hz, 1H), 4.20 (s, 3H), 3.87 (d, J = 12.8 Hz, 2H), 3.04 (t, J = 11.6 Hz, 2H), 2.71-2.65 (m, 1H), 2.60 (q, J = 7.1 Hz, 2H), 2.35-2.28 (m, 6H), 1.95 (d, J = 11.9 Hz, 2H), 1.44 (t, J = 12.2 Hz, 2H), 1.04 (t, J = 7.1 Hz, 3H). |
| 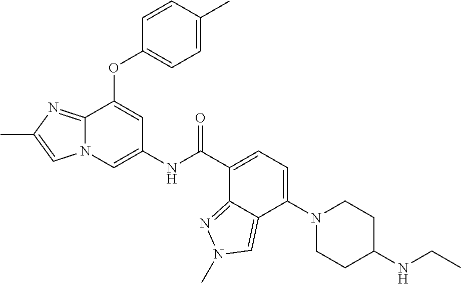 [SMILES: CCNC1CCN(c2ccc(C(=O)Nc3cc(Oc4ccc(C)cc4)c4nc(C)cn4c3)c3nn(C)cc23)CC1] Compound 328 | 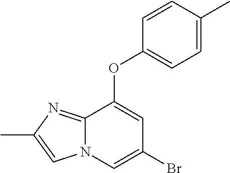 C51 | The crude product was purified by Prep-HPLC (Condition 16, Gradient 1) to afford 4-(4-(ethylamino)- piperidin-1-yl)-2-methyl-N- (2-methyl-8-(p-tolyloxy)- imidazo[1,2-a]pyridin-6-yl)- 2H-indazole-7-carboxamide 2,2,2-trifluoroacetate (Compound 328, 80 mg, 122.8 μmol, 71% yield) as a solid. LCMS (ES, m/z): 538 [M + H]⁺. ¹H NMR (400 MHz, DMSO-d₆) δ 11.10 (s, 1H), 9.31 (d, J = 1.6 Hz, 1H), 8.84 (s, 1H), 8.64 (s, 2H), 8.21 (s, 1H), 7.94 (d, J = 8.1 Hz, 1H), 7.40-7.32 (m, 3H), 7.28-7.20 (m, 2H), 6.53 (d, J = 8.2 Hz, 1H), 4.20 (s, 3H), 4.03 (d, J = 12.8 Hz, 2H), 3.43-3.22 (m, 1H), 3.03 (dd, J = 13.7, 9.4 Hz, 4H), 2.46 (s, 3H), 2.37 (s, 3H), 2.19-2.10 (m, 2H), 1.70 (qd, J = 12.0, 3.9 Hz, 2H), 1.23 (t, J = 7.2 Hz, 3H). |
| 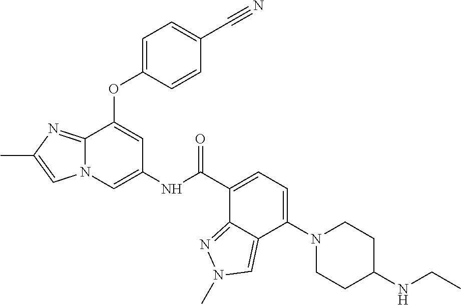 [SMILES: CCNC1CCN(c2ccc(C(=O)Nc3cc(Oc4ccc(C#N)cc4)c4nc(C)cn4c3)c3nn(C)cc23)CC1] Compound 352 | 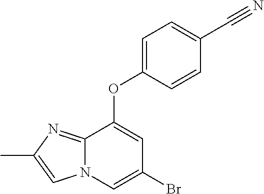 C52 | The crude product was purified by Prep-HPLC (Condition 14, Gradient 2) to afford N-[8-(4- cyanophenoxy)-2-methyl- imidazo[1,2-a]pyridin-6-yl]-4- [4-(ethylamino)-1-piperidyl]- 2-methylindazole-7- carboxamide (Compound 352, 35 mg, 63.8 μmol, 32% yield) as a solid. LCMS (ES, m/z): 549 [M + H]⁺. ¹H NMR (400 MHz, DMSO-d₆) δ 11.07 (s, 1H), 9.33 (d, J = 1.7 Hz, 1H), 8.76 (s, 1H), 7.97 (d, J = 8.1 Hz, 1H), 7.91-7.80 (m, 3H), 7.29 (d, J = 1.7 Hz, 1H), 7.23-7.15 (m, 2H), 6.49 (d, J = 8.3 Hz, 1H), 4.25 (s, 3H), 3.88 (d, J = 12.9 Hz, 2H), 3.11-3.00 (m, 2H), 2.68 (d, J = 4.0 Hz, 1H), 2.61 (q, J = 7.1 Hz, 2H), 2.27 (s, 3H), 2.00-1.92 (m, 2H), 1.50-1.37 (m, 2H), 1.04 (t, J = 7.1 Hz, 3H). |
| 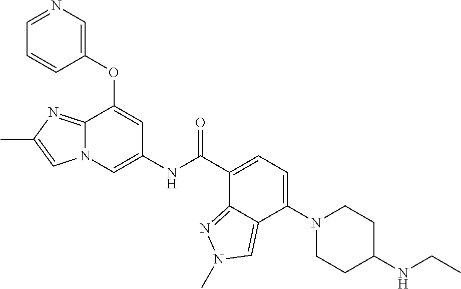 [SMILES: CCNC1CCN(c2ccc(C(=O)Nc3cc(Oc4cccnc4)c4nc(C)cn4c3)c3nn(C)cc23)CC1] Compound 353 | 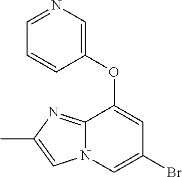 C53 | The crude product was purified by Prep-HPLC (Condition 14, Gradient 4) to afford 4-[4-(ethylamino)-1- piperidyl]-2-methyl-N-[2- methyl-8-(3-pyridyloxy)- imidazo[1,2-a]-pyridin-6-yl]- indazole-7-carboxamide (Compound 353, 30 mg, 57.2 μmol, 45% yield) as a solid. LCMS (ES, m/z): 525 [M + H]⁺. ¹H NMR (400 MHz, DMSO-d₆) δ 11.02 (s, 1H), 9.23 (d, J = 1.7 Hz, 1H), 8.75 (s, 1H), 8.47 (d, J = 2.8 Hz, 1H), 8.38 (dd, J = 4.5, 1.4 Hz, 1H), 7.95 (d, J = 8.1 Hz, 1H), 7.87 (d, J = 1.0 Hz, 1H), 7.49 (ddd, J = 8.4, 2.9, 1.4 Hz, 1H), 7.42 (dd, J = 8.4, 4.6 Hz, 1H), 7.11 (d, J = 1.7 Hz, 1H), 6.48 (d, J = 8.2 Hz, 1H), 4.23 (s, 3H), 3.92-3.83 (m, 2H), 3.10- 2.99 (m, 2H), 2.68 (q, J = 5.1, 4.6 Hz, 1H), 2.60 (q, J = 7.1 Hz, 2H), 2.28 (s, 3H), 1.95 (dd, J = 13.2, 3.7 Hz, 2H), 1.51- 1.35 (m, 2H), 1.04 (t, J = 7.1 Hz, 3H). |

An analogous method was followed beginning with B98 in place of B9, to obtain the following compounds.

| Compound | Starting Material | Characterization |
|---|---|---|
| 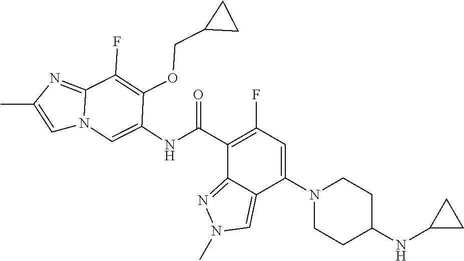 [SMILES: Cc1cn2cc(NC(=O)c3c(F)cc(N4CCC(NC5CC5)CC4)c4cn(C)nc34)c(OCC3CC3)c(F)c2n1] Compound 321 | 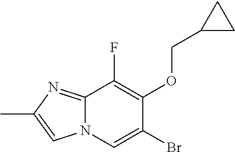 C60 | The residue was purified by reversed-phase flash chromatography (Condition 3, Gradient 5) to afford 4-[4- (cyclopropylamino)-1- piperidyl]-N-[7- (cyclopropylmethoxy)-8-fluoro- 2-methyl-imidazo[1,2-a]- pyridin-6-yl]-6-fluoro-2-methyl- indazole-7-carboxamide (Compound 321, 60 mg, 109.2 μmol, 59% yield) as a solid. LCMS (ES, m/z): 550 [M + H]⁺. ¹H NMR (400 MHz, DMSO-d6) δ 11.40 (s, 1H), 9.46 (d, J = 1.2 Hz, 1H), 8.83 (s, 1H), 7.76 (d, J = 3.1 Hz, 1H), 6.24 (d, J = 16.2 Hz, 1H), 4.24-4.18 (m, 5H), 3.92 (d, J = 13.0 Hz, 2H), 3.19-3.08 (m, 2H), 2.81 (dt, J = 9.9, 5.6 Hz, 1H), 2.34-2.29 (m, 3H), 2.12 (tt, J = 6.7, 3.6 Hz, 1H), 1.99 (d, J = 12.7 Hz, 2H), 1.44 (q, J = 9.9 Hz, 2H), 1.30 (ddd, J = 12.6, 8.1, 4.9 Hz, 1H), 0.51-0.36 (m, 4H), 0.24 (ddt, J = 8.0, 6.5, 4.2 Hz, 4H). |
| 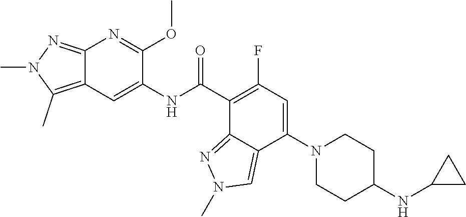 [SMILES: COc1nc2nn(C)c(C)c2cc1NC(=O)c1c(F)cc(N2CCC(NC3CC3)CC2)c2cn(C)nc12] Compound 418 | 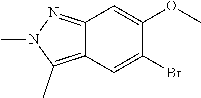 C173 | Modification: Step 1 was ran at 80° C. for 16 h. The crude product was purified by Prep-HPLC (Condition 5, Gradient 14) to afford 4-[4- (cyclopropylamino)-1- piperidyl]-6-fluoro-N-(6- methoxy-2,3-dimethyl-indazol- 5-yl)-2-methyl-indazole-7- carboxamide (Compound 418, 35 mg, 69.2 μmol, 40% yield) as a solid. LCMS (ES, m/z): 506 [M + H]⁺. ¹H NMR (400 MHz, DMSO-d₆) δ 11.55 (s, 1H), 8.76 (s, 1H), 8.69 (s, 1H), 6.94 (s, 1H), 6.19 (d, J = 16.1 Hz, 1H), 4.21 (s, 3H), 3.97 (d, J = 14.5 Hz, 6H), 3.87 (d, J = 12.8 Hz, 2H), 3.13- 3.02 (m, 2H), 2.77 (d, J = 10.9 Hz, 1H), 2.52 (s, 3H), 2.10 (dq, J = 6.8, 3.5 Hz, 1H), 2.01-1.93 (m, 2H), 1.43 (d, J = 11.4 Hz, 2H), 0.38 (td, J = 6.3, 4.0 Hz, 2H), 0.26-0.18 (m, 2H). |

### Example 99: Synthesis of Compound 329

### Synthesis of B85

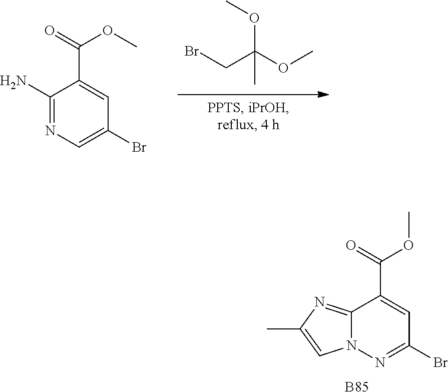

To a stirred mixture of methyl 2-amino-5-bromo-pyridine-3-carboxylate (3 g, 12.98 mmol) and 1-bromo-2,2-dimethoxy-propane (2.38 g, 12.98 mmol) in iPrOH (30 mL) was added PPTs (1.63 g, 6.49 mmol) in portions at room temperature under N₂. The resulting mixture was stirred for 4 h at 80° C. under N₂. The mixture was allowed to cool down to room temperature. The resulting mixture was concentrated under reduced pressure. The residue was adjusted PH to 8 with saturated aqueous NaHCO₃. The resulting mixture was extracted with EtOAc (3×50 mL). The combined organics were washed with brine (10 mL), dried over anhydrous Na₂SO₄, and filtered. The filtrate was concentrated under reduced pressure and purified by silica gel column chromatography (33% EtOAc in PE) to afford methyl 6-bromo-2-methyl-imidazo[1,2-a]pyridine-8-carboxylate (3 g, 11.15 mmol, 86% yield) as a solid. LCMS (ES, m/z): 269 [M+H]⁺.

### Synthesis of B86

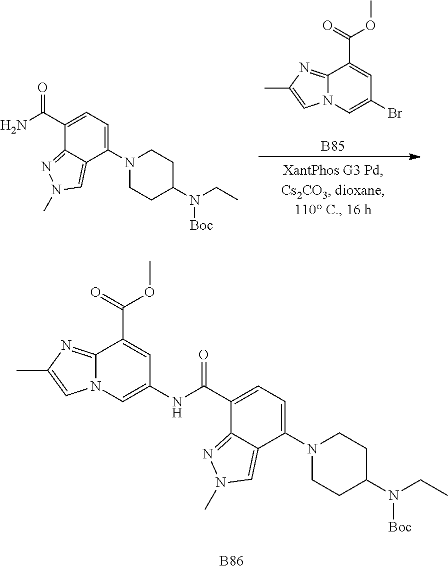 [SMILES: "B86" ; CCN(C(=O)OC(C)(C)C)C1CCN(c2ccc(C(=O)Nc3cc(C(=O)OC)c4nc(C)cn4c3)c3nn(C)cc23)CC1 ; CCN(C(=O)OC(C)(C)C)C1CCN(c2ccc(C(N)=O)c3nn(C)cc23)CC1 ; "B85" ; COC(=O)c1cc(Br)cn2cc(C)nc12]

To a stirred mixture of tert-butyl N-[1-(7-carbamoyl-2-methyl-indazol-4-yl)-4-piperidyl]-N-ethyl-carbamate (200 mg, 498.1 μmol) and methyl 6-bromo-2-methyl-imidazo[1,2-a]pyridine-8-carboxylate (161 mg, 597.8 μmol) in 1,4-dioxane (4 mL) was added Cs₂CO₃ (325 mg, 996.3 μmol) and XantPhos Pd G3 (47 mg, 49.81 μmol) in portions at room temperature under N₂. The resulting mixture was stirred for 16 h at 110° C. under N₂. The mixture was allowed to cool down to room temperature. The resulting mixture was concentrated under reduced pressure and purified by silica gel column chromatography (50% EtOAc in PE) to afford methyl 6-[[4-[4-[tert-butoxycarbonyl(ethyl)amino]-1-piperidyl]-2-methyl-indazole-7-carbonyl]amino]-2-methyl-imidazo[1,2-a]pyridine-8-carboxylate (75 mg, 127.2 μmol, 26% yield) as a solid. LCMS (ES, m/z): 590 [M+H]⁺.

### Synthesis of B87

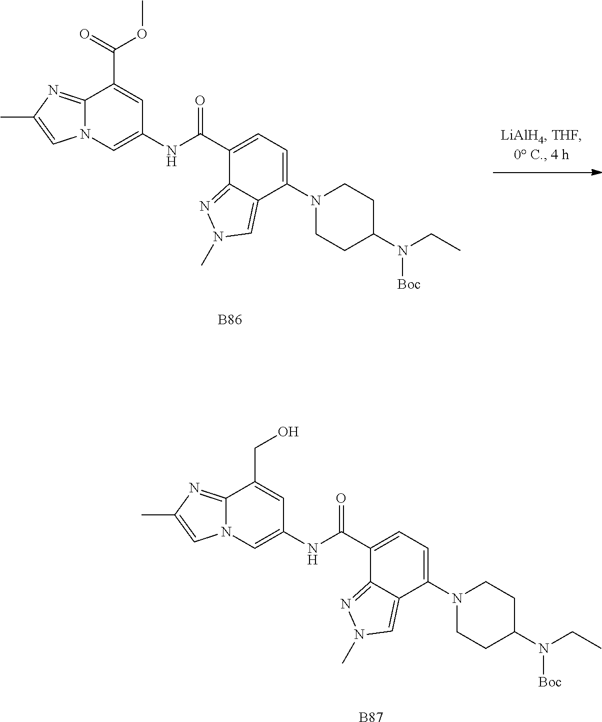

To a stirred mixture of methyl 6-[[4-[4-[tert-butoxycarbonyl(ethyl)amino]-1-piperidyl]-2-methyl-indazole-7-carbonyl]amino]-2-methyl-imidazo[1,2-a]pyridine-8-carboxylate (60 mg, 101.8 μmol) in THF (2 mL) was added Lithium aluminum hydride (8 mg, 203.5 μmol) in portions at 0° C. The resulting mixture was stirred for 4 h at 0° C. under N₂. The reaction was quenched by the addition of water (5 mL) at room temperature. The resulting mixture was extracted with EtOAc (3×20 mL). The combined organic layers were washed with brine (10 mL), dried over anhydrous Na₂SO₄, and filtered. The filtrate was concentrated under reduced pressure, to afford tert-butyl N-ethyl-N-[1-[7-[[8-(hydroxymethyl)-2-methyl-imidazo[1,2-a]pyridin-6-yl]carbamoyl]-2-methyl-indazol-4-yl]-4-piperidyl]carbamate (40 mg, 71.2 μmol, 70% yield) as a solid. LCMS (ES, m/z): 562 [M+H]⁺.

### Synthesis of Compound 329

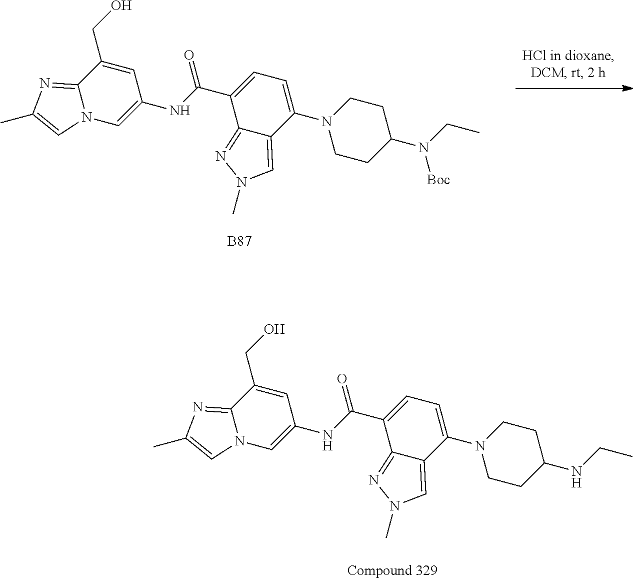

To a stirred mixture of tert-butyl N-ethyl-N-[1-[7-[[8-(hydroxymethyl)-2-methyl-imidazo[1,2-a]pyridin-6-yl]carbamoyl]-2-methyl-indazol-4-yl]-4-piperidyl]carbamate (40 mg, 71.22 μmol) in DCM (2 mL) was added HCl (4.0 M in 1,4-dioxane, 0.5 mL), and the reaction was stirred for 2 h at room temperature. The resulting mixture was concentrated under reduced pressure and purified by reverse flash chromatography (Condition 5, Gradient 1) to afford 4-[4-(ethylamino)-1-piperidyl]-N-[8-(hydroxymethyl)-2-methyl-imidazo[1,2-a]pyridin-6-yl]-2-methyl-indazole-7-carboxamide (Compound 329, 4.5 mg, 9.75 μmol, 14% yield) as a solid. LCMS (ES, m/z): 462 [M+H]⁺. ¹H NMR (400 MHz, Methanol-d₄) δ 9.63 (d, J=1.9 Hz, 1H), 8.59 (s, 1H), 8.16 (d, J=8.1 Hz, 1H), 8.05-8.00 (m, 1H), 7.94 (d, J=1.7 Hz, 1H), 6.63 (d, J=8.1 Hz, 1H), 5.00 (s, 2H), 4.37 (s, 3H), 4.15 (d, J=13.1 Hz, 2H), 3.48-3.37 (m, 1H), 3.20 (q, J=7.3 Hz, 2H), 3.13 (t, J=12.6 Hz, 2H), 2.60-2.56 (m, 3H), 2.29 (d, J=11.5 Hz, 2H), 1.93-1.80 (m, 2H), 1.38 (t, J=7.3 Hz, 3H).

### Example 100: Synthesis of Compound 360

### Synthesis of B88

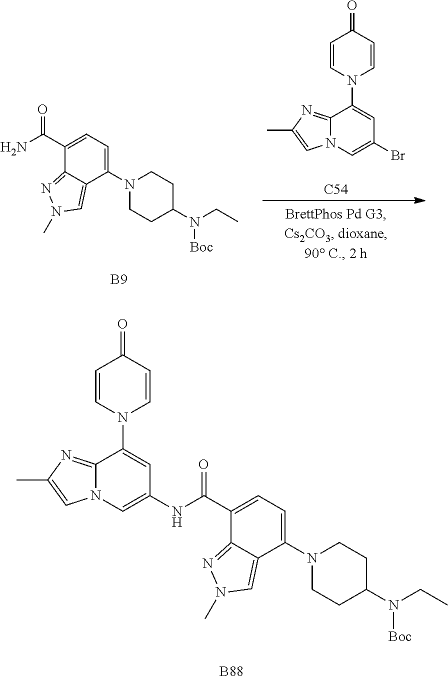

To a stirred mixture of tert-butyl N-[1-(7-carbamoyl-2-methylindazol-4-yl)-4-piperidyl]-N-ethyl-carbamate (0.11 g, 274 μmol) and 1-(6-bromo-2-methylimidazo[1,2-a]pyridin-8-yl)-pyridin-4-one (100 mg, 328.8 μmol) in Dioxane (3 mL) were added Cs₂CO₃ (178 mg, 547.9 μmol), BrettPhos Pd G3 (25 mg, 27.4 μmol), and the reaction was stirred for 2 h at 90° C. under N₂. The resulting mixture was filtered, concentrated under reduced pressure, and purified by silica gel column chromatography (89% EtOAc in PE) to afford tert-butyl N-ethyl-N-[1-[2-methyl-7-[[2-methyl-8-(4-oxo-1-pyridyl)imidazo[1,2-a]pyridin-6-yl]carbamoyl]indazol-4-yl]-4-piperidyl]carbamate (B88, 0.08 g, 128.1 μmol, 47% yield) as a solid. LCMS (ES, m/z): 625 [M+H]⁺.

### Synthesis of Compound 360

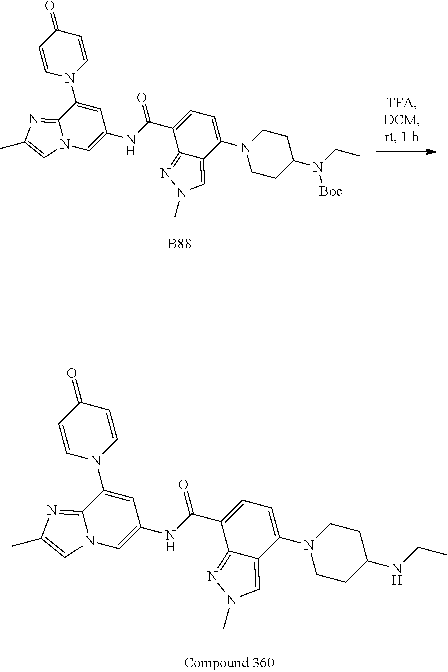

To a solution of tert-butyl N-ethyl-N-[1-[2-methyl-7-[[2-methyl-8-(4-oxo-1-pyridyl)-imidazo[1,2-a]pyridin-6-yl]carbamoyl]indazol-4-yl]-4-piperidyl]carbamate (0.08 g, 128.1 μmol) in DCM (1 mL) was added TFA (0.4 mL), and the reaction was stirred for 1 h at rt. The resulting mixture was concentrated under reduced pressure and adjusted to pH 8 with saturated NaHCO₃ (aq.). The resulting mixture was filtered, concentrated under reduced pressure, and purified by reversed-phase flash chromatography (Condition 3, Gradient 12) to afford 4-(4-(ethylamino)piperidin-1-yl)-2-methyl-N-(2-methyl-8-(4-oxopyridin-1-(4H)-yl)imidazo[1,2-a]-pyridin-6-yl)-2H-indazole-7-carboxamide (Compound 360, 0.03 g, 57.2 μmol, 45% yield) as a solid. LCMS (ES, m/z): 525 [M+H]⁺. ¹H NMR (400 MHz, DMSO-d₆) δ 11.12 (s, 1H), 9.45 (d, J=1.8 Hz, 1H), 8.78 (s, 1H), 8.22-8.13 (m, 2H), 8.02-7.95 (m, 2H), 7.57 (d, J=1.8 Hz, 1H), 6.50 (d, J=8.2 Hz, 1H), 6.34-6.26 (m, 2H), 4.29 (s, 3H), 3.89 (d, J=12.9 Hz, 2H), 3.07 (t, J=11.4 Hz, 2H), 2.68 (s, 1H), 2.61 (q, J=7.1 Hz, 2H), 2.36 (s, 3H), 1.96 (d, J=12.1 Hz, 2H), 1.49-1.38 (m, 2H), 1.04 (t, J=7.1 Hz, 3H).

An analogous method as followed to obtain the following compound.

| | Starting | |
|---|---|---|
| Compound | Material | Characterization |
| 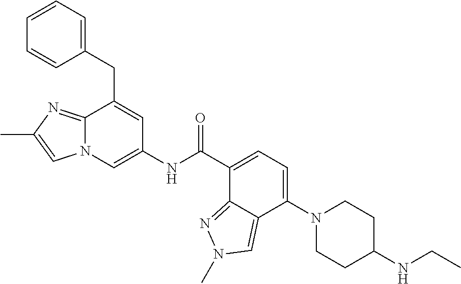 [SMILES: CCNC1CCN(c2ccc(C(=O)Nc3cc(Cc4ccccc4)c4nc(C)cn4c3)c3nn(C)cc23)CC1] Compound 338 | 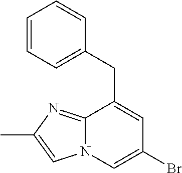 C90 | Modification: Step 1 was ran for 16 h. Step 2 was ran for 2 h. The crude product was purified by Prep-HPLC (Condition 5, Gradient 11) to afford N-(8-benzyl-2- methylimidazo-[1,2-a]pyridin-6-yl)- 4-[4-(ethylamino)-1-piperidyl]-2- methyl-indazole-7-carboxamide (Compound 338, 24 mg, 58 μmol, 45% yield) as a solid. LCMS (ES, m/z): 522 [M + H]⁺. ¹H NMR (400 MHz, DMSO-d₆) δ 10.98 (s, 1H), 9.21 (d, J = 2.0 Hz, 1H), 8.76 (s, 1H), 7.95 (d, J = 8.1 Hz, 1H), 7.77 (d, J = 1.0 Hz, 1H), 7.45-7.37 (m, 2H), 7.33 (dd, J = 8.4, 6.8 Hz, 2H), 7.27-7.19 (m, 1H), 6.92 (d, J = 2.0 Hz, 1H), 6.48 (d, J = 8.1 Hz, 1H), 4.26 (s, 2H), 4.23 (s, 3H), 3.87 (d, J = 12.9 Hz, 2H), 3.04 (t, J = 11.7 Hz, 2H), 2.72-2.65 (m, 1H), 2.61 (q, J = 7.1 Hz, 2H), 2.34 (s, 3H), 1.96 (d, J = 12.7 Hz, 2H), 1.43 (q, J = 9.9 Hz, 2H), 1.04 (t, J = 7.1 Hz, 3H). |
| 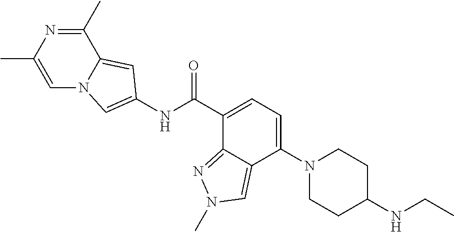 [SMILES: CCNC1CCN(c2ccc(C(=O)Nc3cc4c(C)nc(C)cn4c3)c3nn(C)cc23)CC1] Compound 345 | 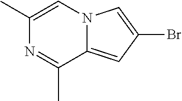 | Modification: Step 1 was ran at 110° C. for 16 h. Step 2 was ran using HCl in dioxane (0.5 mL) and DCM (0.6 mL), room temperature for 2 h. The residue was purified by reversed- phase flash chromatography (Condition 3, Gradient 3) to afford N- (1,3-dimethylpyrrolo[1,2-a]pyrazin- 7-yl)-4-[4-(ethylamino)-1-piperidyl]- 2-methyl-indazole-7-carboxamide (Compound 345, 35 mg, 78.55 μmol, 39% yield) as a solid. LCMS (ES, m/z): 446 [M + H]⁺. ¹H NMR (400 MHz, DMSO-d₆) δ 11.21 (s, 1H), 8.76 (s, 1H), 8.19 (d, J = 1.5 Hz, 1H), 8.01- 7.94 (m, 2H), 6.83 (t, J = 1.3 Hz, 1H), 6.48 (d, J = 8.2 Hz, 1H), 4.30 (s, 3H), 3.88 (dd, J = 10.4, 6.2 Hz, 2H), 3.09- 2.98 (m, 2H), 2.68 (q, J = 6.1, 4.7 Hz, 1H), 2.61 (q, J = 7.1 Hz, 2H), 2.55 (s, 3H), 2.28 (s, 3H), 1.96 (d, J = 12.3 Hz, 2H), 1.51-1.38 (m, 2H), 1.04 (t, J = 7.1 Hz, 3H). |

An analogous method was followed, beginning with B98, to obtain the following compounds.

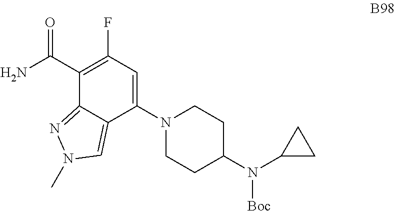 [SMILES: Cn1cc2c(N3CCC(N(C(=O)OC(C)(C)C)C4CC4)CC3)cc(F)c(C(N)=O)c2n1]

| | Starting | |
|---|---|---|
| Compound | Material | Characterization |
| 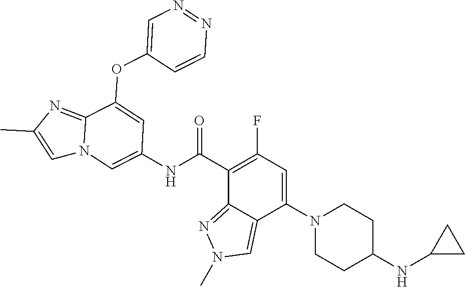 [SMILES: Cc1cn2cc(NC(=O)c3c(F)cc(N4CCC(NC5CC5)CC4)c4cn(C)nc34)cc(Oc3ccnnc3)c2n1] Compound 386 | 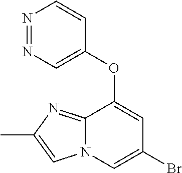 | Modification: Step 1 was ran at 90° C. for 5 h. Step 2 was ran for 2 h. The mixture was purified by reversed- phase flash chromatography (Condition 3, Gradient 15) to afford 4- [4-(cyclopropylamino)-1-piperidyl]-6- fluoro-2-methyl-N-(2-methyl-8- pyridazin-4-yloxy-imidazo-[1,2-a]- pyridin-6-yl)indazole-7-carboxamide (Compound 386, 0.005 g, 9 μmol, 9% yield) as a solid. LCMS (ES, m/z): 556 [M + H]⁺. ¹H NMR (400 MHz, Methanol-d₄) δ 9.22 (d, J = 1.7 Hz, 1H), 9.16 (dd, J = 3.1, 0.9 Hz, 1H), 8.99 (dd, J = 6.1, 0.9 Hz, 1H), 8.48 (s, 1H), 7.74 (d, J = 1.0 Hz, 1H), 7.39 (d, J = 1.8 Hz, 1H), 7.16 (dd, J = 6.1, 3.1 Hz, 1H), 6.23 (d, J= 15.9 Hz, 1H), 4.21 (s, 3H), 4.00 (d, J = 13.1 Hz, 2H), 3.09 (t, J = 11.8 Hz, 2H), 2.96-2.85 (m, 1H), 2.35 (d, J = 0.9 Hz, 3H), 2.24 (tt, J = 7.0, 3.8 Hz, 1H), 2.14 (d, J = 11.8 Hz, 2H), 1.57 (qd, J = 12.3, 3.9 Hz, 2H), 0.54 (td, J = 6.7, 4.6 Hz, 2H), 0.50-0.37 (m, 2H). |
| 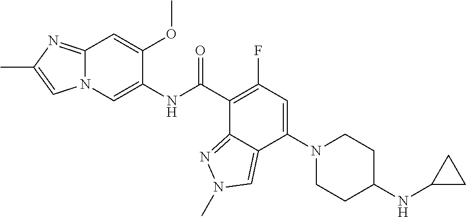 [SMILES: COc1cc2nc(C)cn2cc1NC(=O)c1c(F)cc(N2CCC(NC3CC3)CC2)c2cn(C)nc12] Compound 395 | 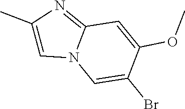 | Modification: Step 1 was ran for 5 h. Step 2 was ran for 2 h. The mixture was purified by reversed- phase flash chromatography (Condition 3, Gradient 10) to afford 4- [4-(cyclopropylamino)-1-piperidyl]-6- fluoro-N-(7-methoxy-2-methyl- imidazo[1,2-a]pyridin-6-yl)-2-methyl- indazole-7-carboxamide (Compound 395, 31.7 mg, 64.5 μmol, 38% yield) as a solid. LCMS (ES, m/z): 492 [M + H]⁺. ¹H NMR (400 MHz, DMSO-d₆) δ 11.59 (s, 1H), 9.53 (s, 1H), 8.80 (s, 1H), 7.54 (s, 1H), 6.95 (s, 1H), 6.22 (d, J = 16.1 Hz, 1H), 4.23 (s, 3H), 4.05 (s, 3H), 3.91 (d, J = 12.9 Hz, 2H), 3.13 (t, J = 11.9 Hz, 2H), 2.80 (d, J = 5.1 Hz, 1H), 2.27 (s, 3H), 2.12 (tt, J = 6.6, 3.8 Hz, 1H), 1.99 (d, J = 12.8 Hz, 2H), 1.48-1.42 (m, 2H), 0.40 (dt, J = 6.2, 3.0 Hz, 2H), 0.24 (p, J = 4.0 Hz, 2H). |
| 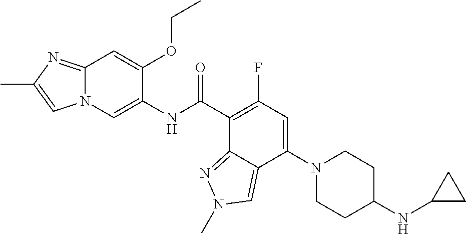 [SMILES: CCOc1cc2nc(C)cn2cc1NC(=O)c1c(F)cc(N2CCC(NC3CC3)CC2)c2cn(C)nc12] Compound 406 | 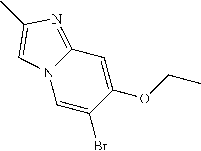 C160 | Modification: Step 2 was ran for 2 h. The residue was purified by reversed- phase flash chromatography (Condition 3, Gradient 8) to afford 4- [4-(cyclopropylamino)-1-piperidyl]- N-(7-ethoxy-2-methyl-imidazo[1,2- a]pyridin-6-yl)-6-fluoro-2-methyl- indazole-7-carboxamide (Compound 406, 0.02 g, 39.6 μmol, 22% yield) as a solid. LCMS (ES, m/z): 506 [M + H]⁺. ¹H NMR (400 MHz, DMSO-d₆) δ 11.16 (s, 1H), 9.57 (s, 1H), 8.79 (s, 1H), 7.53-7.48 (m, 1H), 6.91 (s, 1H), 6.21 (d, J = 16.2 Hz, 1H), 4.25 (q, J = 6.9 Hz, 2H), 4.20 (s, 3H), 3.88 (d, J = 13.1 Hz, 2H), 3.16-3.04 (m, 2H), 2.82- 2.78 (m, 1H), 2.26-2.21 (m, 3H), 2.09 (tt, J = 6.7, 3.6 Hz, 1H), 1.96 (d, J = 12.3 Hz, 2H), 1.53 (t, J = 7.0 Hz, 3H), 1.42 (q, J = 13.1, 11.5 Hz, 2H), 0.42- 0.33 (m, 2H), 0.25-0.17 (m, 2H). |
| 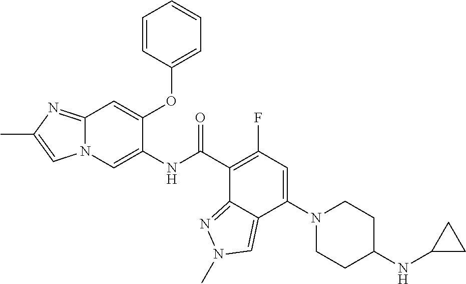 [SMILES: Cc1cn2cc(NC(=O)c3c(F)cc(N4CCC(NC5CC5)CC4)c4cn(C)nc34)c(Oc3ccccc3)cc2n1] Compound 417 | 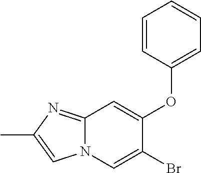 C171 | Modification: Step 1 was ran for 12 h. Step 2 was ran for 2 h. The resulting mixture was purified by reversed-phase flash chromatography (Condition 3, Gradient 22) to afford 4- [4-(cyclopropylamino)-1-piperidyl]-6- fluoro-2-methyl-N-(2-methyl-7- phenoxyimidazo[1,2-a]pyridin-6-yl)- indazole-7-carboxamide (Compound 417, 13 mg, 23.5 μmol, 15% yield) as a solid. LCMS (ES, m/z): 554 [M + H]⁺. ¹H NMR (400 MHz, DMSO-d₆) δ 11.72 (s, 1H), 9.69 (s, 1H), 8.74 (s, 1H), 7.70 (s, 1H), 7.54-7.45 (m, 2H), 7.32 (d, J = 1.3 Hz, 1H), 7.31-7.23 (m, 2H), 6.73 (s, 1H), 6.22 (d, J = 16.2 Hz, 1H), 3.95-3.86 (m, 5H), 3.12 (t, J = 12.0 Hz, 2H), 2.80 (s, 1H), 2.27 (s, 3H), 2.10-2.09 (m, 1H), 1.97 (d, J = 12.8 Hz, 2H), 1.45-1.40 (m, 2H), 0.43- 0.36 (m, 2H), 0.23 (d, J = 3.4 Hz, 2H). |
| 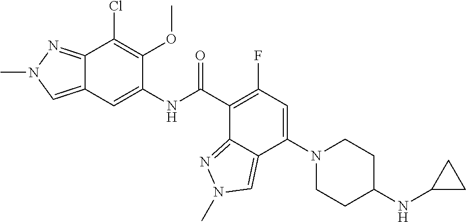 [SMILES: COc1c(NC(=O)c2c(F)cc(N3CCC(NC4CC4)CC3)c3cn(C)nc23)cc2cn(C)nc2c1Cl] Compound 433 | 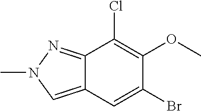 C175 | Modification: Step 1 was ran at 70° C. for 2 h. Step 2 was ran using HCl in dioxane and DCM at room temperature for 2 h. The residue was purified by reversed- phase flash chromatography (Condition 3, Gradient 15) to afford N- (7-chloro-6-methoxy-2-methyl- indazol-5-yl)-4-[4- (cyclopropylamino)-1-piperidyl]-6- fluoro-2-methyl-indazole-7- carboxamide (Compound 433, 0.03 g, 57 μmol, 36% yield) as a solid. LCMS (ES, m/z): 526 [M + H]⁺. ¹H NMR (400 MHz, DMSO-d₆) δ 11.57 (s, 1H), 8.82 (s, 2H), 8.40 (s, 1H), 6.24 (d, J = 16.2 Hz, 1H), 4.25 (s, 3H), 4.16 (s, 3H), 4.03 (s, 3H), 3.92 (d, J = 13.2 Hz, 2H), 3.18-3.07 (m, 2H), 2.82 (s, 1H), 2.14 (dd, J = 7.1, 3.6 Hz, 1H), 2.00 (d, J = 12.2 Hz, 2H), 1.46 (q, J = 9.8 Hz, 2H), 0.41 (dt, J= 6.2, 3.0 Hz, 2H), 0.29-0.21 (m, 2H). |
| 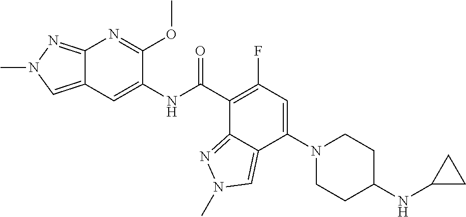 [SMILES: COc1nc2nn(C)cc2cc1NC(=O)c1c(F)cc(N2CCC(NC3CC3)CC2)c2cn(C)nc12] Compound 431 | 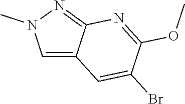 C178 | Modification: Step 1 was ran for 16 h. The residue was purified by reversed- phase flash chromatography (Condition 3, Gradient 18) to afford 4- [4-(cyclopropylamino)-1-piperidyl]-6- fluoro-N-(6-methoxy-2-methyl- pyrazolo-[3,4-b]pyridin-5-yl)-2- methyl-indazole-7-carboxamide (Compound 431, 35 mg, 71.1 μmol, 42% yield) as a solid. LCMS (ES, m/z): 493 [M + H]⁺. ¹H NMR (400 MHz, DMSO-d₆) δ 11.71 (s, 1H), 9.04 (s, 1H), 8.81 (s, 1H), 8.19 (s, 1H), 6.22 (d, J = 16.2 Hz, 1H), 4.25 (s, 3H), 4.13 (s, 3H), 4.07 (s, 3H), 3.91 (d, J = 13.1 Hz, 2H), 3.12 (t, J = 11.3 Hz, 2H), 2.80 (dt, J = 9.9, 5.5 Hz, 1H), 2.11 (tq, J = 7.4, 3.7 Hz, 1H), 1.99 (d, J = 12.4 Hz, 2H), 1.48-1.39 (m, 2H), 0.44-0.36 (m, 2H), 0.27-0.19 (m, 2H). |
| 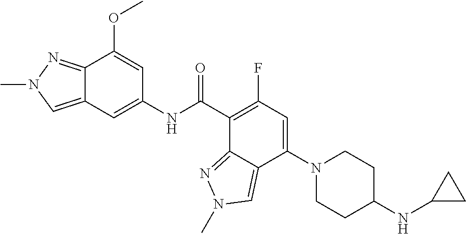 [SMILES: COc1cc(NC(=O)c2c(F)cc(N3CCC(NC4CC4)CC3)c3cn(C)nc23)cc2cn(C)nc12] Compound 423 | 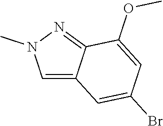 | Modification: Step 1 was ran at 70° C. Step 2 was ran using HCl in dioxane at room temperature for 2 h. The residue was purified by reversed- phase flash chromatography (Condition 3, Gradient 22) to afford 4- [4-(cyclopropylamino)-1-piperidyl]-6- fluoro-N-(7-methoxy-2-methyl- indazol-5-yl)-2-methyl-indazole-7- carboxamide (Compound 423, 0.05 g, 101.7 μmol, 40% yield) as a solid. LCMS (ES, m/z):492 [M + H]⁺. ¹H NMR (400 MHz, DMSO-d₆) δ 10.79 (s, 1H), 8.72 (s, 1H), 8.21 (s, 1H), 7.78 (d, J = 1.5 Hz, 1H), 6.87 (d, J = 1.7 Hz, 1H), 6.22 (d, J = 15.1 Hz, 1H), 4.20 (s, 3H), 4.12 (s, 3H), 3.93 (s, 3H), 3.84 (d, J = 13.0 Hz, 2H), 3.10-2.99 (m, 2H), 2.81-2.73 (m, 1H), 2.26 (s, 1H), 2.12 (tt, J = 6.7, 3.6 Hz, 1H), 2.03-1.95 (m, 2H), 1.49 (d, J = 9.7 Hz, 1H), 1.45 (s, 1H), 0.45-0.34 (m, 2H), 0.27-0.19 (m, 2H). |
| 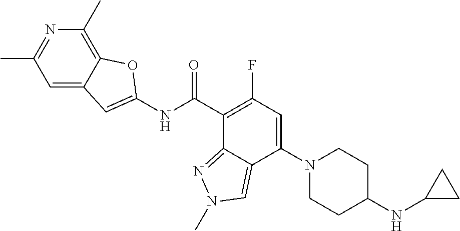 [SMILES: Cc1cc2cc(NC(=O)c3c(F)cc(N4CCC(NC5CC5)CC4)c4cn(C)nc34)oc2c(C)n1] Compound 456 | 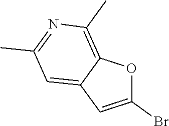 | Modification: Step 1 was ran at 90° C. for 3 h. Step 2 was ran using HCl in dioxane and DCM at room temperature for 2 h. The residue was purified by reversed- phase flash chromatography (Condition 3, Gradient 11) to afford 4- [4-(cyclopropylamino)-1-piperidyl]- N-(5,7-dimethylfuro-[2,3-c]pyridin-2- yl)-6-fluoro-2-methyl-indazole-7- carboxamide (Compound 456, 40 mg, 83.94 μmol, 61% yield) as a solid. LCMS (ES, m/z): 477 [M + H]⁺. ¹H NMR (400 MHz, DMSO-d₆) δ 12.01 (s, 1H), 8.82 (s, 1H), 7.18 (s, 1H), 6.73 (s, 1H), 6.24 (d, J = 16.1 Hz, 1H), 4.20 (s, 3H), 3.92 (d, J = 13.2 Hz, 2H), 3.22- 3.04 (m, 2H), 2.79 (dt, J = 9.8, 5.5 Hz, 1H), 2.55 (s, 3H), 2.43 (s, 3H), 2.29 (d, J = 15.9 Hz, 1H), 2.08 (m, 1H), 1.96 (d, J = 12.8 Hz, 2H), 1.41 (q, J = 12.1, 11.1 Hz, 2H), 0.37 (m, 2H), 0.24 -0.15 (m, 2H). ¹⁹F NMR (376 MHz, DMSO- d₆) δ −106.43. |
| 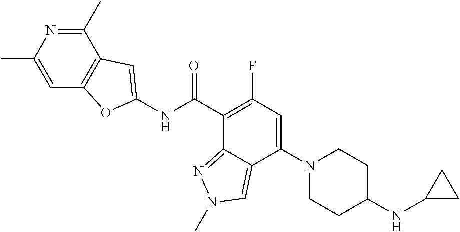 [SMILES: Cc1cc2oc(NC(=O)c3c(F)cc(N4CCC(NC5CC5)CC4)c4cn(C)nc34)cc2c(C)n1] Compound 491 | 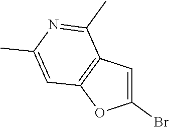 D49 | Modification: Step 1 was ran at 100° C. for 24 h. Step 2 was for 1 h. The residue was purified by reversed- phase flash chromatography (Condition 3, Gradient 26) to afford 4- [4-(cyclopropylamino)-1-piperidyl]- N-(4,6-dimethyl-furo[3,2-c]pyridin-2- yl)-6-fluoro-2-methyl-indazole-7- carboxamide (Compound 491, 17.6 mg, 35.3 μmol). LCMS (ES, m/z): 477 [M + H]⁺. ¹H NMR (400 MHz, DMSO- d₆) δ 11.91 (s, 1H), 8.84 (s, 1H), 7.27 (s, 1H), 6.82 (d, J = 1.1 Hz, 1H), 6.27 (d, J = 15.9 Hz, 1H), 4.22 (s, 3H), 3.94 (d, J = 13.1 Hz, 2H), 3.17 (t, J = 11.8 Hz, 2H), 2.86-2.77 (m, 1H), 2.59 (s, 3H), 2.50 (s, 3H), 2.36-2.29 (m, 1H), 2.15-2.08 (m, 1H), 1.99 (d, J = 12.1 Hz, 2H), 1.44 (q, J = 10.3, 9.6 Hz, 2H), 0.44-0.36 (m, 2H), 0.27-0.19 (m, 2H). |

An analogous method was followed to obtain the following compounds.

| Compound | Starting Material | Characterization |
|---|---|---|
| 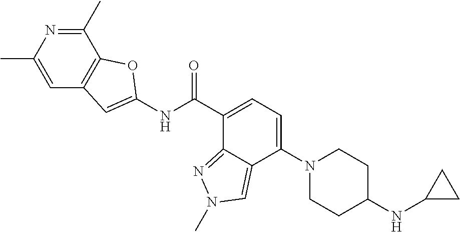 [SMILES: Cc1cc2cc(NC(=O)c3ccc(N4CCC(NC5CC5)CC4)c4cn(C)nc34)oc2c(C)n1] Compound 451 | 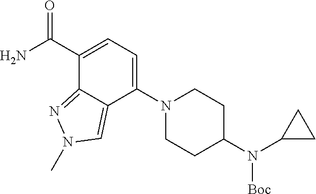 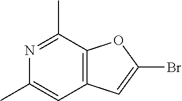 C184 | Modification: Step 1 was ran for 6 h at 90° C. Step 2 was ran for 1 h. The residue was purified by reversed-phase flash chromatography (Condition 4, Gradient 5) to afford 4-(4- (cyclopropylamino)piperidin- 1-yl)-N-(5,7-dimethyl-furo- [2,3-c]pyridin-2-yl)-2-methyl- 2H-indazole-7-carboxamide 2,2,2-trifluoroacetate (Compound 451, 50 mg, 87.3 μmol, 49% yield) as a solid. LCMS (ES, m/z): 459 [M + H]⁺. ¹H NMR (400 MHz, Methanol-d₄) δ 8.66 (s, 1H), 8.21 (d, J = 8.2 Hz, 1H), 7.66 (s, 1H), 7.22 (s, 1H), 6.66 (d, J = 8.2 Hz, 1H), 4.37 (s, 3H), 4.24 (d, J = 13.3 Hz, 2H), 3.66-3.56 (m, 1H), 3.22 (t, J = 12.6 Hz, 2H), 2.91 (s, 3H), 2.87 (dq, J = 7.5, 3.7 Hz, 1H), 2.73 (s, 3H), 2.37 (d, J = 11.8 Hz, 2H), 2.00-1.78 (m, 2H), 1.02 (q, J = 6.9, 6.1 Hz, 2H), 0.97-0.88 (m, 2H). |
| 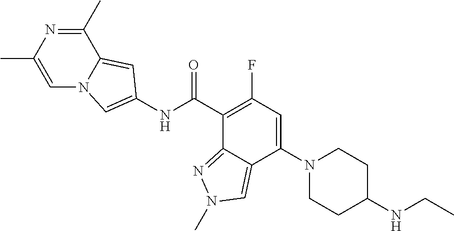 [SMILES: CCNC1CCN(c2cc(F)c(C(=O)Nc3cc4c(C)nc(C)cn4c3)c3nn(C)cc23)CC1] Compound 344 | 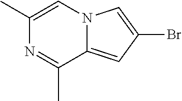 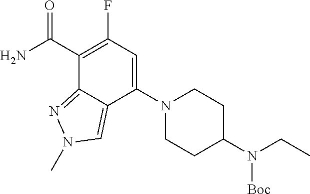 B90 | Modification: Step 1 was ran at 110° C. for 16 h. Step 2 was ran using HCl in dioxane (0.3 mL) and DCM (2 mL), room temperature for 2 h. The residue was purified by reversed-phase flash chromatography (Condition 3, Gradient 3) to afford N-(1,3- dimethylpyrrolo[1,2-a]- pyrazin-7-yl)-4-[4- (ethylamino)-1-piperidyl]-6- fluoro-2-methyl-indazole-7- carboxamide (Compound 344, 25 mg, 53.9 μmol, 30% yield) as a solid. LCMS (ES, m/z): 464 [M + H]⁺. ¹H NMR (400 MHz, DMSO-d₆) δ 11.11 (s, 1H), 8.74 (s, 1H), 8.13 (d, J = 1.4 Hz, 1H), 7.98 (s, 1H), 6.77 (d, J = 1.3 Hz, 1H), 6.22 (d, J = 15.3 Hz, 1H), 4.21 (s, 3H), 3.91-3.81 (m, 2H), 3.11- 3.00 (m, 2H), 2.75-2.60 (m, 2H), 2.59 (d, J = 7.1 Hz, 1H), 2.53 (s, 3H), 2.30-2.25 (m, 3H), 1.95 (dd, J = 13.0, 3.7 Hz, 2H), 1.42 (q, J = 9.7 Hz, 2H), 1.04 (t, J = 7.1 Hz, 3H). |
| 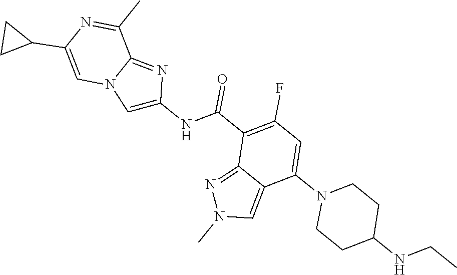 [SMILES: CCNC1CCN(c2cc(F)c(C(=O)Nc3cn4cc(C5CC5)nc(C)c4n3)c3nn(C)cc23)CC1] Compound 346 | 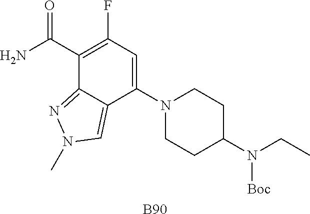 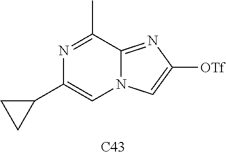 | Modification: Step 1 was ran for 5 h. Step 2 was ran using HCl in dioxane (0.3 mL) and DCM (2 mL), room temperature for 2 h. The residue was purified by Prep-HPLC (Condition 2, Gradient 7) to afford N-(6- cyclopropyl-8-methyl- imidazo[1,2-a]pyrazin-2-yl)- 4-(4-(ethylamino)-piperidin- 1-yl)-6-fluoro-2-methyl-2H- indazole-7-carboxamide bis(2,2,2-trifluoroacetate) (Compound 346, 0.015 g, 20.9 μmol, 18% yield) as a solid. LCMS (ES, m/z): 491 [M + H]⁺. ¹H NMR (300 MHz, Methanol-d₄) δ 8.60 (s, 1H), 8.50 (s, 1H), 8.38 (s, 1H), 6.37 (d, J = 15.7 Hz, 1H), 4.34 (s, 3H), 4.19 (d, J = 13.4 Hz, 2H), 3.52-3.45 (m, 1H), 3.26-3.11 (m, 4H), 2.89 (s, 3H), 2.28 (d, J = 12.1 Hz, 2H), 2.12 (ddd, J = 13.4, 8.3, 5.1 Hz, 1H), 1.91- 1.74 (m, 2H), 1.38 (t, J = 7.3 Hz, 3H), 1.14-0.93 (m, 4H). |
| 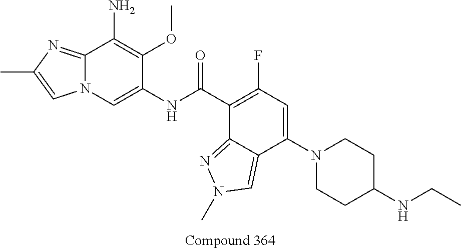 [SMILES: CCNC1CCN(c2cc(F)c(C(=O)Nc3cn4cc(C)nc4c(N)c3OC)c3nn(C)cc23)CC1] | 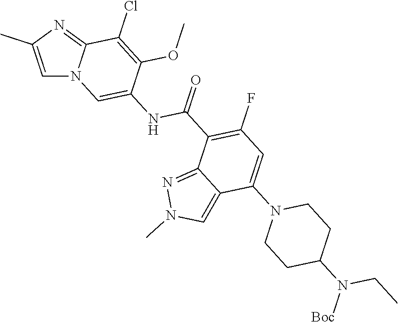 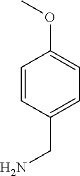 | Modification: Step 1 was ran for 2 h at 120° C. Step 2 was ran using HCl in dioxane (1 mL) and DCM (1 mL), 50° C. for 2 h. The crude product was purified by Prep-HPLC (Condition 5, Gradient 15) to afford N-(8-amino-7- methoxy-2-methyl- imidazo[1,2-a]-pyridin-6-yl)- 4-[4-(ethylamino)-1- piperidyl]-6-fluoro-2-methyl- indazole-7-carboxamide (Compound 364, 2.5 mg, 5.05 μmol, 9% yield) as a solid. LCMS (ES, m/z): 495 [M + H]⁺. ¹H NMR (300 MHz, Methanol-d₄) δ 8.95 (s, 1H), 8.48 (s, 1H), 7.40 (d, J = 1.0 Hz, 1H), 6.21 (d, J = 16.2 Hz, 1H), 4.27 (s, 3H), 3.97 (s, 5H), 3.05 (t, J = 12.3 Hz, 2H), 2.90- 2.61 (m, 3H), 2.37 (d, J = 0.9 Hz, 3H), 2.07 (d, J = 12.7 Hz, 2H), 1.68-1.42 (m, 2H), 1.16 (t, J = 7.2 Hz, 3H). |
| 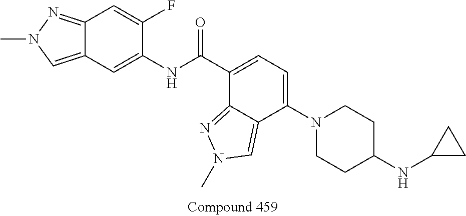 [SMILES: Cn1cc2cc(NC(=O)c3ccc(N4CCC(NC5CC5)CC4)c4cn(C)nc34)c(F)cc2n1] | 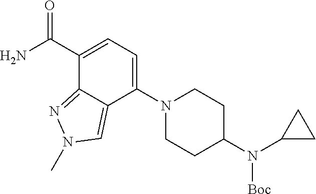 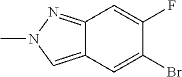 | Modification: Step 1 was ran for 3 h at 90° C. Step 2 was ran using HCl in dioxane and DCM for 1 h at room temperature. The residue was purified by reversed-phase flash chromatography (Condition 3, Gradient 8) to afford 4-[4- (cyclopropylamino)-1- piperidyl]-N-(6-fluoro-2- methyl-indazol-5-yl)-2- methyl-indazole-7- carboxamide (Compound 459, 0.05 g, 43.3 μmol, 51% yield) as a solid. LCMS (ES, m/z): 462 [M + H]⁺. ¹H NMR (400 MHz, DMSO-d₆) δ 11.41 (d, J = 3.5 Hz, 1H), 8.83 (d, J = 7.9 Hz, 1H), 8.77 (s, 1H), 8.36 (s, 1H), 8.01 (d, J = 8.1 Hz, 1H), 7.52 (d, J = 12.1 Hz, 1H), 6.49 (d, J = 8.2 Hz, 1H), 4.25 (s, 3H), 4.14 (s, 3H), 3.88 (d, J = 12.8 Hz, 2H), 3.11-3.00 (m, 2H), 2.78 (dt, J = 10.0, 5.7 Hz, 1H), 2.26 (s, 1H), 2.12 (tt, J = 6.7, 3.6 Hz, 1H), 2.01 (d, J = 12.3 Hz, 2H), 1.49 (s, 1H), 1.47 (s, 1H), 0.40 (dt, J = 6.1, 3.0 Hz, 2H), 0.27-0.20 (m, 2H). |
| 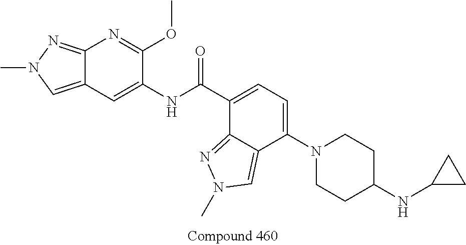 [SMILES: COc1nc2nn(C)cc2cc1NC(=O)c1ccc(N2CCC(NC3CC3)CC2)c2cn(C)nc12] | 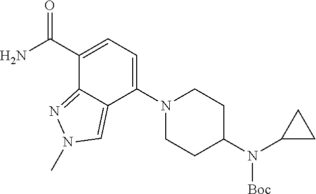 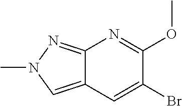 | Modification: Step 2 was ran using HCl in dioxane and DCM at room temperature for 2 h. The precipitated solids were collected by filtration and purified by trituration with MeOH to afford 4-[4- (cyclopropylamino)-1- piperidyl]-N-(6-methoxy-2- methyl-pyrazolo[3,4-b]- pyridin-5-yl)-2-methyl- indazole-7-carboxamide (Compound 460, 30 mg, 63.2 μmol, 66% yield) as a solid. LCMS (ES, m/z): 475 [M + H]⁺. ¹H NMR (400 MHz, DMSO-d₆) δ 11.63 (s, 1H), 9.06 (s, 1H), 8.74 (s, 1H), 8.18 (s, 1H), 7.96 (d, J = 8.1 Hz, 1H), 6.46 (d, J = 8.2 Hz, 1H), 4.27 (s, 3H), 4.13 (s, 3H), 4.05 (s, 3H), 3.85 (dd, J = 10.7, 6.6 Hz, 2H), 3.08-2.97 (m, 2H), 2.75 (s, 1H), 2.23 (s, 1H), 2.10 (tt, J = 6.7, 3.6 Hz, 1H), 1.98 (dd, J = 13.5, 3.4 Hz, 2H), 1.56-1.34 (m, 2H), 0.38 (td, J = 6.3, 4.0 Hz, 2H), 0.25-0.17 (m, 2H). |
| 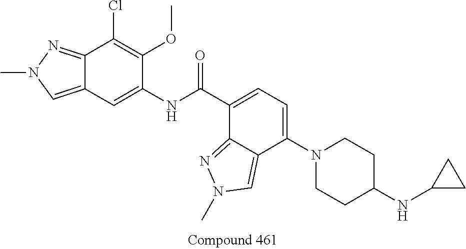 [SMILES: COc1c(NC(=O)c2ccc(N3CCC(NC4CC4)CC3)c3cn(C)nc23)cc2cn(C)nc2c1Cl] | 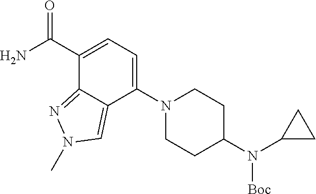 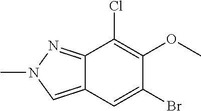 | Modification: Step 1 was ran for 3 h. Step 2 was ran for 0.5 h. The mixture was purified by reversed-phase flash chromatography (Condition 3, Gradient 27) to afford N-(7- chloro-6-methoxy-2-methyl- indazol-5-yl)-4-[4- (cyclopropylamino)-1- piperidyl]-2-methyl-indazole- 7-carboxamide (Compound 461, 0.035 g, 28% yield) as a solid. LCMS (ES, m/z): 508 [M + H]⁺. ¹H NMR (400 MHz, DMSO-d₆) δ 11.57 (s, 1H), 8.89 (s, 1H), 8.77 (s, 1H), 8.41 (s, 1H), 8.02 (d, J = 8.1 Hz, 1H), 6.50 (d, J = 8.2 Hz, 1H), 4.30 (s, 3H), 4.17 (s, 3H), 4.06 (s, 3H), 3.88 (d, J = 12.8 Hz, 2H), 3.05 (t, J = 11.4 Hz, 2H), 2.18-2.06 (m, 1H), 2.01 (d, J = 12.4 Hz, 1H), 2.91 (d, J = 12.2 Hz, 2H), 1.51-1.46 (m, 1H), 0.40 (dt, J = 6.1, 3.0 Hz, 2H), 0.28-0.20 (m, 2H). |
| 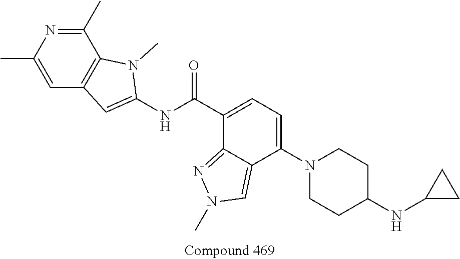 [SMILES: Cc1cc2cc(NC(=O)c3ccc(N4CCC(NC5CC5)CC4)c4cn(C)nc34)n(C)c2c(C)n1] | 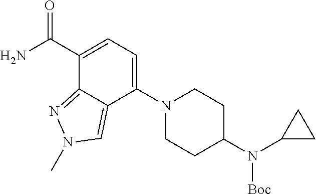 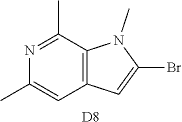 | The reaction mixture was concentrated under reduced pressure, and the crude product was purified by Prep- HPLC (Condition 5, Gradient 11) to afford 4-[4- (cyclopropyl-amino)-1- piperidyl]-2-methyl-N-(1,5,7- trimethyl-pyrrolo[2,3-c]- pyridin-2-yl)indazole-7- carboxamide (Compound 469, 20 mg, 42.4 μmol). LCMS (ES, m/z): 472 [M + H]⁺. ¹H NMR (400 MHz, DMSO-d₆) δ 11.62 (s, 1H), 8.83 (s, 1H), 8.01 (d, J = 8.2 Hz, 1H), 7.09 (s, 1H), 6.75 (s, 1H), 6.52 (d, J = 8.3 Hz, 1H), 4.28 (s, 3H), 4.09 (s, 3H), 3.92 (d, J = 12.8 Hz, 2H), 3.10 (t, J = 11.8 Hz, 2H), 2.85 (s, 3H), 2.80 (p, J = 5.5 Hz, 1H), 2.41 (s, 3H), 2.13 (tt, J = 6.7, 3.6 Hz, 1H), 2.01 (d, J = 12.7 Hz, 2H), 1.48 (q, J = 13.1, 12.0 Hz, 2H), 0.41 (td, J = 6.3, 4.0 Hz, 2H), 0.28-0.20 (m, 2H). |
| 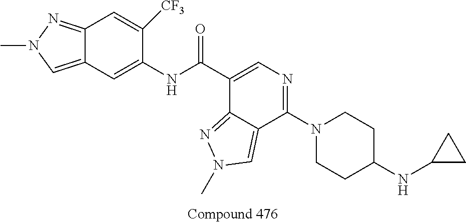 [SMILES: Cn1cc2cc(NC(=O)c3cnc(N4CCC(NC5CC5)CC4)c4cn(C)nc34)c(C(F)(F)F)cc2n1] | 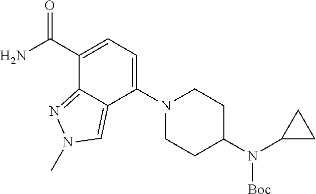 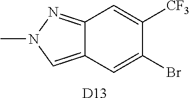 | Modification: Step 1 was ran for 5 h. Step 2 was ran using HCl in dioxane and DCM at room temperature for 1 h. The reaction mixture was purified by reversed-phase flash chromatography (Condition 3, Gradient 20) to afford 4-[4-(cyclopropyl- amino)-1-piperidyl]-2- methyl-N-[2-methyl-6- (trifluoromethyl)-indazol-5- yl]indazole-7-carboxamide (Compound 476, 0.03 g, 58.7 μmol, 33% yield) as a solid. LCMS (ES, m/z): 512 [M + H]⁺. ¹H NMR (300 MHz, DMSO-d₆) δ 11.04 (s, 1H), 8.75 (s, 1H), 8.52 (s, 1H), 8.44 (s, 1H), 8.12 (s, 1H), 8.00 (d, J = 8.0 Hz, 1H), 6.48 (d, J = 8.2 Hz, 1H), 4.25 (s, 3H), 4.19 (s, 3H), 3.88 (d, J = 12.7 Hz, 2H), 3.05 (t, J = 11.8 Hz, 2H), 2.78 (s, 1H), 2.26 (s, 1H), 2.13 (s, 1H), 2.01 (d, J = 12.7 Hz, 2H), 1.52-1.45 (m, 1H), 0.40 (dt, J = 6.1, 3.0 Hz, 2H), 0.24 (p, J = 3.9 Hz, 2H). |
| 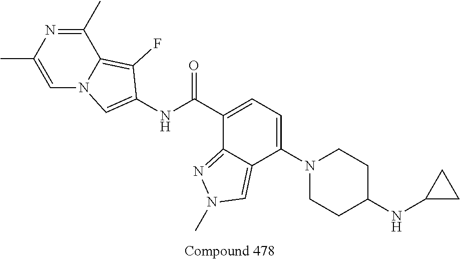 [SMILES: Cc1cn2cc(NC(=O)c3ccc(N4CCC(NC5CC5)CC4)c4cn(C)nc34)c(F)c2c(C)n1] | 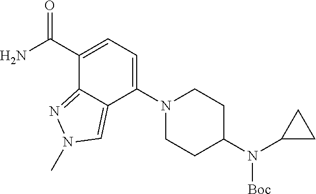 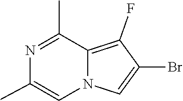 | Modification: Step 1 was ran for 5 h. Step 2 was ran for 1 h. The residue was purified by reversed-phase flash chromatography (Condition 3, Gradient 34) to afford 4-[4- (cyclopropyl-amino)-1- piperidyl]-N-(8-fluoro-1,3- dimethyl-pyrrolo[1,2-a]- pyrazin-7-yl)-2-methyl- indazole-7- carboxamide (Compound 478, 29.7 mg, 62.5 μmol). LCMS (ES, m/z): 476 [M + H]⁺. ¹H NMR (300 MHz, DMSO-d₆) δ 11.26 (s, 1H), 8.78 (s, 1H), 8.15 (d, J = 3.6 Hz, 1H), 7.99 (d, J = 8.1 Hz, 1H), 7.90 (s, 1H), 6.49 (d, J = 8.2 Hz, 1H), 4.25 (s, 3H), 3.88 (d, J = 12.6 Hz, 2H), 3.06 (t, J = 11.9 Hz, 2H), 2.76 (d, J = 10.7 Hz, 1H), 2.62 (s, 3H), 2.27 (s, 1H), 2.24 (s, 3H), 2.12 (dt, J = 6.7, 3.1 Hz, 1H), 2.00 (d, J = 12.6 Hz, 2H), 1.51-1.44 (d, J = 10.5 Hz, 1H), 0.39 (dt, J = 6.1, 2.9 Hz, 2H), 0.23 (p, J = 3.9 Hz, 2H). |
| 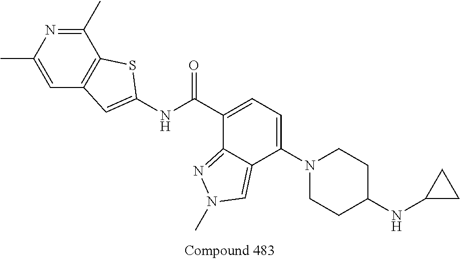 [SMILES: Cc1cc2cc(NC(=O)c3ccc(N4CCC(NC5CC5)CC4)c4cn(C)nc34)sc2c(C)n1] | 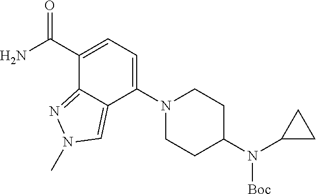 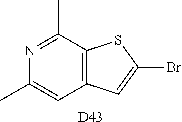 | Modification: Step 2 was ran using HCl in dioxane and DCM. The crude product was purified by Prep-HPLC (Condition 14, Gradient 5) to afford 4-[4-(cyclopropyl- amino)-1-piperidyl]-N-(5,7- dimethyl-thieno[2,3-c]- pyridin-2-yl)-2-methyl- indazole-7-carboxamide (Compound 483, 50 mg, 105.4 μmol). LCMS (ES, m/z): 475 [M + H]⁺. ¹H NMR (400 MHz, DMSO-d₆) δ 12.14 (s, 1H), 8.83 (s, 1H), 8.00 (d, J = 8.2 Hz, 1H), 7.31 (s, 1H), 7.14 (s, 1H), 6.51 (d, J = 8.3 Hz, 1H), 4.30 (s, 3H), 3.93 (d, J = 12.8 Hz, 2H), 3.18-3.07 (m, 2H), 2.80 (dt, J = 10.0, 5.8 Hz, 1H), 2.62 (s, 3H), 2.49 (s, 3H), 2.33 (s, 1H), 2.12 (tt, J = 6.7, 3.6 Hz, 1H), 2.00 (d, J = 12.4 Hz, 2H), 1.46 (d, J = 11.1 Hz, 2H), 0.44-0.36 (m, 2H), 0.27-0.19 (m, 2H). |
| 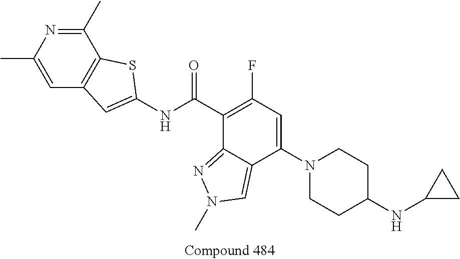 [SMILES: Cc1cc2cc(NC(=O)c3c(F)cc(N4CCC(NC5CC5)CC4)c4cn(C)nc34)sc2c(C)n1] | 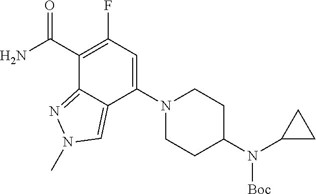 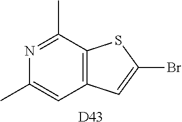 | Modification: Step 1 was ran for 5 h. Step 2 was ran for 1 h. The crude product was purified by Prep-HPLC (Condition 5, Gradient 11) to afford 4-[4-(cyclopropyl- amino)-1-piperidyl]-N-(5,7- dimethyl-thieno[2,3- c]pyridin-2-yl)-6-fluoro-2- methyl-indazole-7- carboxamide (Compound 484, 20 mg, 40.6 μmol). LCMS (ES, m/z): 475 [M + H]⁺. ¹H NMR (400 MHz, DMSO-d₆) δ 12.23 (s, 1H), 8.82 (s, 1H), 7.30 (s, 1H), 7.08 (s, 1H), 6.27 (d, J = 15.7 Hz, 1H), 4.23 (s, 3H), 3.93 (d, J = 13.1 Hz, 2H), 3.21-3.10 (m, 2H), 2.87-2.74 (m, 1H), 2.62 (s, 3H), 2.49 (s, 3H), 2.33 (s, 1H), 2.11 (tt, J = 6.7, 3.6 Hz, 1H), 2.04-1.95 (m, 2H), 1.44 (q, J = 9.6 Hz, 2H), 0.40 (dt, J = 6.2, 3.0 Hz, 2H), 0.27-0.19 (m, 2H). |
| 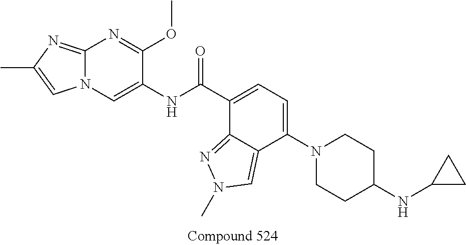 [SMILES: COc1nc2nc(C)cn2cc1NC(=O)c1ccc(N2CCC(NC3CC3)CC2)c2cn(C)nc12] | 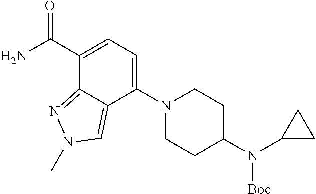 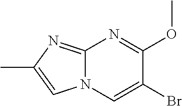 | Modification: Step 1 was ran at 100° C. for 3 h. Step 2 was ran for 1 h. The residue was purified by reversed-phase flash chromatography (Condition 3, Gradient 42) to afford 4-[4- (cyclopropyl-amino)-1- piperidyl]-N-(7-methoxy-2- methylimidazo-[1,2-a]- pyrimidin-6-yl)-2-methyl- indazole-7-carboxamide (Compound 524, 0.04 g, 84.3 μmol). LCMS (ES, m/z): 475 [M + H]⁺. ¹H NMR (400 MHz, DMSO-d₆) δ 11.50 (s, 1H), 9.74 (s, 1H), 8.79 (s, 1H), 7.98 (d, J = 8.1 Hz, 1H), 7.52 (d, J = 1.1 Hz, 1H), 6.49 (d, J = 8.2 Hz, 1H), 4.29 (s, 3H), 4.16 (s, 3H), 3.90 (d, J = 12.7 Hz, 2H), 3.08 (t, J = 11.6 Hz, 2H), 2.78 (s, 1H), 2.27 (s, 3H), 2.13 (dq, J = 6.6, 3.4 Hz, 1H), 2.01 (d, J = 12.3 Hz, 2H), 1.49-1.45 (d, J = 10.0 Hz, 1H), 0.45-0.36 (m, 2H), 0.24 (p, J = 3.9 Hz, 2H). |
| 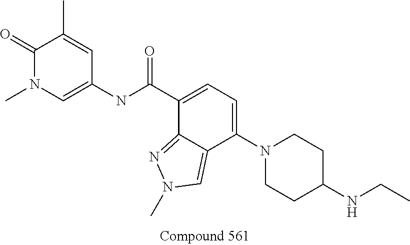 [SMILES: CCNC1CCN(c2ccc(C(=O)Nc3cc(C)c(=O)n(C)c3)c3nn(C)cc23)CC1] | 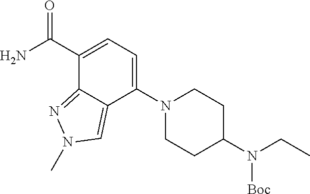 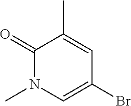 | Modification: Step 1 was ran for 16 h. Step 2 was ran for 1 h. The residue was purified by reversed-phase flash chromatography (Condition 3, Gradient 26) to afford N-(1,5- dimethyl-6-oxo-3-pyridyl)-4- [4-(ethylamino)-1-piperidyl]- 2-methyl-indazole-7- carboxamide (Compound 561, 26.1 mg, 61.8 μmol). LCMS (ES, m/z): 423 [M + H]⁺. ¹H NMR (400 MHz, DMSO-d₆) δ 10.63 (s, 1H), 8.74 (s, 1H), 8.26 (d, J = 2.8 Hz, 1H), 7.92 (d, J = 8.1 Hz, 1H), 7.49 (dd, J = 3.0, 1.4 Hz, 1H), 6.46 (dd, J = 8.2, 1.9 Hz, 1H), 4.26 (s, 3H), 3.85 (d, J = 12.6 Hz, 2H), 3.48 (s, 3H), 3.02 (t, J = 11.8 Hz, 2H), 2.70- 2.54 (m, 3H), 2.07 (s, 3H), 1.99-1.91 (m, 2H), 1.56-1.55 (s, 1H), 1.49-1.37 (m, 2H), 1.04 (t, J = 7.1 Hz, 3H). |
| 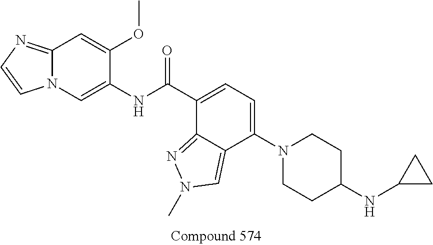 [SMILES: COc1cc2nccn2cc1NC(=O)c1ccc(N2CCC(NC3CC3)CC2)c2cn(C)nc12] | 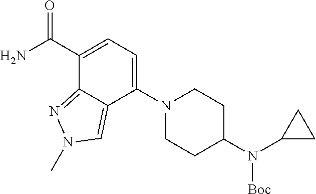 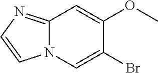 | Modification: Step 1 was ran for 6 h. The residue was purified by reversed-phase flash chromatography (Condition 3, Gradient 26) to afford 4-[4- (cyclopropylamino)-1- piperidyl]-N-(7-methoxy- imidazo[1,2-a]pyridin-6-yl)- 2-methylindazole-7- carboxamide (Compound 574, 20.7 mg, 42.8 μmol). LCMS (ES, m/z): 460 [M + H]⁺. ¹H NMR (400 MHz, DMSO-d₆) δ 11.56 (s, 1H), 9.67 (s, 1H), 8.77 (s, 1H), 7.99 (d, J = 8.1 Hz, 1H), 7.86 (s, 1H), 7.38 (s, 1H), 7.10 (s, 1H), 6.49 (d, J = 8.2 Hz, 1H), 4.29 (s, 3H), 4.10 (s, 3H), 3.89 (d, J = 12.9 Hz, 2H), 3.07 (t, J = 11.6 Hz, 2H), 2.85-2.78 (s, 1H), 2.30-2.29 (m, 1H), 2.13 (dt, J = 6.5, 3.2 Hz, 1H), 2.01 (d, J = 12.6 Hz, 2H), 1.48 (q, J = 9.8 Hz, 2H), 0.40 (dt, J = 6.2, 3.0 Hz, 2H), 0.24 (p, J = 3.9 Hz, 2H). |
| 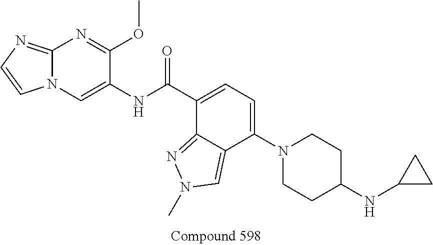 [SMILES: COc1nc2nccn2cc1NC(=O)c1ccc(N2CCC(NC3CC3)CC2)c2cn(C)nc12] | 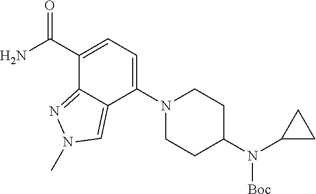 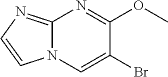 D116 | Modification: Step 1 was ran at 100° C. for 5 h. The crude product was purified by Prep- HPLC (Condition 5, Gradient 12a) to afford 4-[4- (cyclopropylamino)-1- piperidyl]-N-(7-methoxy- imidazo[1,2-a]pyrimidin-6- yl)-2-methyl-indazole-7- carboxamide (Compound 598, 20 mg, 43.4 μmol). LCMS (ES, m/z): 461 [M + H]⁺. ¹H NMR (400 MHz, DMSO-d₆) δ 11.53 (s, 1H), 9.82 (s, 1H), 8.77 (s, 1H), 7.97 (d, J = 8.1 Hz, 1H), 7.78 (d, J = 1.5 Hz, 1H), 7.36 (d, J = 1.5 Hz, 1H), 6.47 (d, J = 8.2 Hz, 1H), 4.27 (s, 3H), 4.16 (s, 3H), 3.88 (d, J = 12.8 Hz, 2H), 3.12-3.00 (m, 2H), 2.76 (dt, J = 10.1, 5.7 Hz, 1H), 2.25 (s, 1H), 2.10 (tt, J = 6.8, 3.6 Hz, 1H), 2.02-1.94 (m, 2H), 1.45 (d, J = 11.2 Hz, 2H), 0.37 (dt, J = 6.2, 3.0 Hz, 2H), 0.25-0.17 (m, 2H). |
| 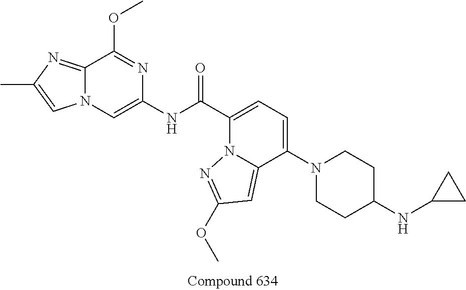 [SMILES: COc1cc2c(N3CCC(NC4CC4)CC3)ccc(C(=O)Nc3cn4cc(C)nc4c(OC)n3)n2n1] | 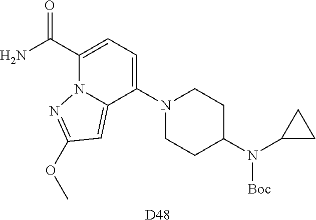 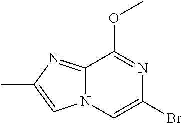 | The crude product was purified by Prep-HPLC (Condition 5, Gradient 30) to afford 4-[4-(cyclopropyl- amino)-1-piperidyl]-2- methoxy-N-(8-methoxy-2- methylimidazo[1,2-a]pyrazin- 6-yl)-pyrazolo[1,5-a]pyridine- 7-carboxamide (Compound 634, 18.5 mg, 38.7 μmol). LCMS (ES, m/z): 491 [M + H]⁺. ¹H NMR (400 MHz, CDCl₃) δ 12.99 (s, 1H), 8.96 (s, 1H), 7.83 (d, J = 8.1 Hz, 1H), 7.41 (d, J = 0.9 Hz, 1H), 6.63 (d, J = 8.1 Hz, 1H), 5.98 (s, 1H), 4.22 (s, 3H), 4.13 (s, 3H), 3.79-3.71 (m, 2H), 2.98- 2.87 (m, 3H), 2.48 (d, J = 0.8 Hz, 3H), 2.24 (tt, J = 6.8, 3.6 Hz, 1H), 2.17 (d, J = 12.3 Hz, 2H), 1.67 (q, J = 11.3, 10.5 Hz, 2H), 0.54 (dt, J = 6.3, 2.9 Hz, 2H), 0.46 (s, 2H). |
| 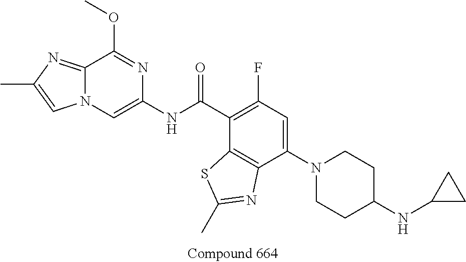 [SMILES: COc1nc(NC(=O)c2c(F)cc(N3CCC(NC4CC4)CC3)c3nc(C)sc23)cn2cc(C)nc12] | 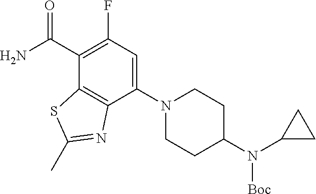 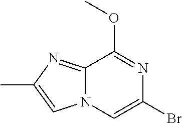 | Modification: Step 1 was ran for 16 h. Step 2 was ran for 1 h. The residue was purified by reversed-phase flash chromatography (Condition 3, Gradient 15) to afford 4-[4- (cyclopropylamino)-1- piperidyl]-6-fluoro-N-(8- methoxy-2-methylimidazo- [1,2-a]pyrazin-6-yl)-2- methyl-1,3-benzothiazole-7- carboxamide (Compound 664, 0.01 g, 19.6 μmol). LCMS (ES, m/z): 510 [M + H]⁺. ¹H NMR (400 MHz, Methanol-d₄) δ 8.89 (s, 1H), 7.66 (d, J = 0.9 Hz, 1H), 6.75 (d, J = 16.5 Hz, 1H), 5.98 (ddt, J = 16.7, 10.2, 6.3 Hz, 1H), 5.29 (dq, J = 17.2, 1.6 Hz, 1H), 5.19 (dd, J = 10.3, 1.6 Hz, 1H), 4.52 (d, J = 12.7 Hz, 2H), 4.13 (s, 3H), 3.37 (dt, J = 6.3, 1.4 Hz, 2H), 3.04 (dd, J = 13.1, 10.8 Hz, 2H), 2.88-2.80 (m, 1H), 2.78 (s, 3H), 2.41 (s, 3H), 2.08 (d, J = 12.9 Hz, 2H), 1.63 (qd, J = 12.1, 3.7 Hz, 2H), 1.32 (s, 1H). |
| 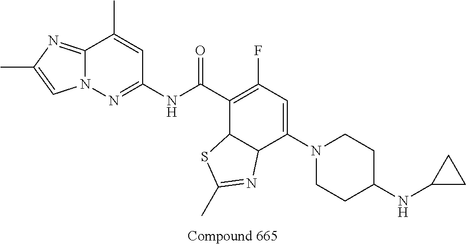 [SMILES: CC1=NC2C(N3CCC(NC4CC4)CC3)=CC(F)=C(C(=O)Nc3cc(C)c4nc(C)cn4n3)C2S1] | 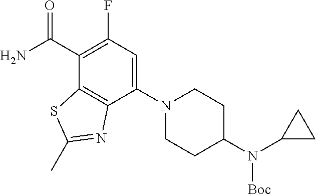 D50 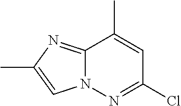 | Modification: Step 2 was ran for 2 h. The crude product was purified by Prep-HPLC (Condition 6, Gradient 11) to afford 4-[4-(cyclopropyl- amino)-1-piperidyl]-N-(2,8- dimethylimidazo[1,2-b]- pyridazin-6-yl)-6-fluoro-2- methyl-1,3-benzothiazole-7- carboxamide (Compound 665, 15.5 mg, 31.4 μmol). LCMS (ES, m/z): 494 [M + H]⁺. ¹H NMR (400 MHz, DMSO-d₆) δ 10.16 (d, J = 7.9 Hz, 1H), 7.91 (s, 1H), 7.74 (s, 1H), 6.84 (d, J = 15.6 Hz, 1H), 4.39 (d, J = 12.5 Hz, 2H), 3.16 (t, J = 11.7 Hz, 2H), 2.76 (s, 3H), 2.76-2.75 (m, 1H), 2.58 (s, 3H), 2.38 (s, 3H), 2.13 (s, 1H), 1.98 (d, J = 13.2 Hz, 2H), 1.50-1.40 (m, 2H), 0.40 (d, J = 5.1 Hz, 2H), 0.23 (s, 2H). |

### Example 101: Synthesis of Compound 299

### Synthesis of B91

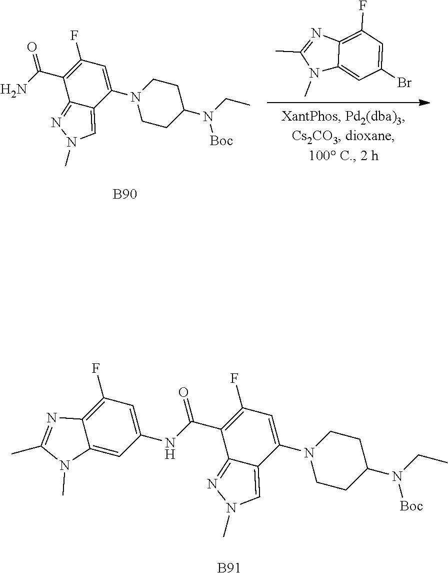 [SMILES: "B90" ; "B91" ; CCN(C(=O)OC(C)(C)C)C1CCN(c2cc(F)c(C(=O)Nc3cc(F)c4nc(C)n(C)c4c3)c3nn(C)cc23)CC1 ; CCN(C(=O)OC(C)(C)C)C1CCN(c2cc(F)c(C(N)=O)c3nn(C)cc23)CC1 ; Cc1nc2c(F)cc(Br)cc2n1C]

To a stirred mixture of tert-butyl N-[1-(7-carbamoyl-6-fluoro-2-methyl-indazol-4-yl)-4-piperidyl]-N-ethyl-carbamate (120 mg, 286.1 μmol) in dioxane (1.5 mL) were added 6-bromo-4-fluoro-1,2-dimethyl-benzimidazole (83.4 mg, 343.3 μmol), Cs₂CO₃ (186.4 mg, 572.1 μmol), Xantphos (33.1 mg, 57.21 μmol) and Pd₂(dba)₃ (26.2 mg, 28.61 μmol), and the reaction was stirred for 2 h at 100° C. under N₂. The resulting mixture was diluted with water (10 mL) and extracted with DCM (2×10 mL). The combined organic layers were washed with water (2×15 mL), dried over anhydrous Na₂SO₄, and filtered. The filtrate was concentrated under reduced pressure and purified by silica gel column chromatography (5% MeOH in DCM) to afford tert-butyl N-ethyl-N-[1-[6-fluoro-7-[(7-fluoro-2,3-dimethyl-benzimidazol-5-yl)carbamoyl]-2-methyl-indazol-4-yl]-4-piperidyl]carbamate (B91, 120 mg, 206.3 μmol, 72% yield) as a solid. LCMS (ES, m/z): 582 [M+H]⁺.

### Synthesis of Compound 299

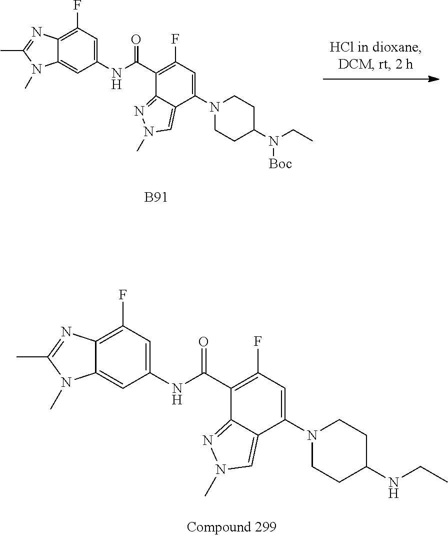

To a solution of tert-butyl N-ethyl-N-[1-[6-fluoro-7-[(7-fluoro-2,3-dimethyl-benzimidazol-5-yl)carbamoyl]-2-methyl-indazol-4-yl]-4-piperidyl]carbamate (110 mg, 189.1 μmol) in DCM (1.5 mL) was added HCl (4.0 M in 1,4-dioxane) (4 M, 400 μL), and the reaction was stirred for 2 h at rt. The resulting mixture was concentrated under reduced pressure and purified by Prep-HPLC (Condition 14, Gradient 2) to afford 4-[4-(ethylamino)-1-piperidyl]-6-fluoro-N-(7-fluoro-2,3-dimethyl-benzimidazol-5-yl)-2-methyl-indazole-7-carboxamide (Compound 299, 45 mg, 93.5 μmol, 49% yield) as a solid. LCMS (ES, m/z): 482 [M+H]⁺. ¹H NMR (400 MHz, DMSO-d₆) δ 11.06 (s, 1H), 8.73 (s, 1H), 7.89 (d, J=1.7 Hz, 1H), 7.27 (dd, J=12.7, 1.6 Hz, 1H), 6.22 (d, J=15.3 Hz, 1H), 4.22 (s, 3H), 3.87 (d, J=12.8 Hz, 2H), 3.73 (s, 3H), 3.07 (t, J=11.4 Hz, 2H), 2.75-2.65 (m, 1H), 2.61 (q, J=7.1 Hz, 2H), 2.54 (s, 3H), 2.00-1.91 (m, 2H), 1.42 (d, J=11.3 Hz, 2H), 1.04 (t, J=7.1 Hz, 3H).

### Example 102: Synthesis of Compound 300

### Synthesis of B92

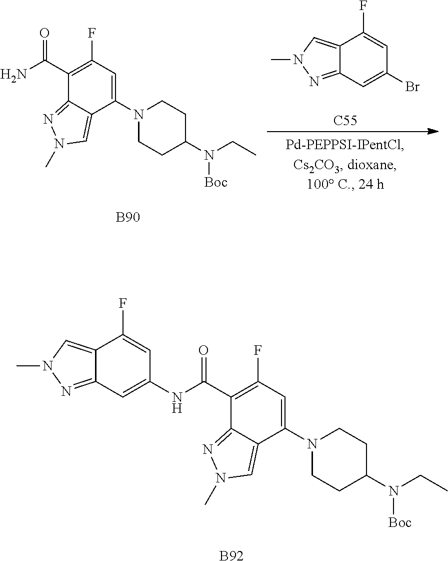 [SMILES: "C55" ; "B90" ; "B92" ; CCN(C(=O)OC(C)(C)C)C1CCN(c2cc(F)c(C(=O)Nc3cc(F)c4cn(C)nc4c3)c3nn(C)cc23)CC1 ; CCN(C(=O)OC(C)(C)C)C1CCN(c2cc(F)c(C(N)=O)c3nn(C)cc23)CC1 ; Cn1cc2c(F)cc(Br)cc2n1]

To a stirred mixture of tert-butyl N-[1-(7-carbamoyl-6-fluoro-2-methyl-indazol-4-yl)-4-piperidyl]-N-ethyl-carbamate (110 mg, 262.2 μmol) and 6-bromo-4-fluoro-2-methyl-2H-indazole (90 mg, 393.3 μmol) in Dioxane (4 mL) were added Cs₂CO₃ (256 mg, 786.7 μmol) and Pd-PEPPSI-IPentCl (21 mg, 26.22 μmol) at room temperature under N₂. The resulting mixture was stirred for 24 h at 100° C. under N₂. The mixture was allowed to cool down to room temperature, concentrated under reduced pressure and purified by silica gel column chromatography (67% EtOAc in PE) to afford tert-butyl N-ethyl-N-[1-[6-fluoro-7-[(4-fluoro-2-methyl-indazol-6-yl)carbamoyl]-2-methyl-indazol-4-yl]-4-piperidyl]carbamate (B92, 60 mg, 105.7 μmol, 40% yield) as a solid. LCMS (ES, m/z): 558 [M+H]⁺.

### Synthesis of Compound 300

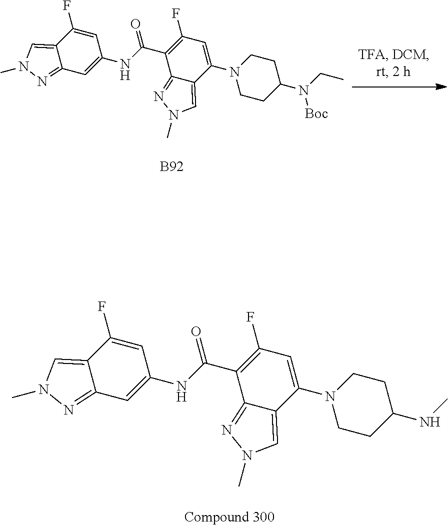

To a solution of tert-butyl N-ethyl-N-[1-[6-fluoro-7-[(4-fluoro-2-methylindazol-6-yl)-carbamoyl]-2-methyl-indazol-4-yl]-4-piperidyl]carbamate (55 mg, 96.89 μmol) in DCM (2 mL) was added TFA (0.2 mL), and the reaction was stirred for 2 h at rt. The resulting mixture was concentrated under reduced pressure and adjusted to pH 8 with saturated NaHCO₃ (aq.). The resulting mixture was diluted with water (10 mL) and extracted with DCM/MeOH (20/1) (3×10 mL). The combined organic layers were washed with brine (10 mL) and dried over anhydrous Na₂SO₄, and filtered. The filtrate was concentrated under reduced pressure and purified by reversed-phase flash chromatography (Condition 3, Gradient 14) to afford 4-[4-(ethylamino)-1-piperidyl]-6-fluoro-N-(4-fluoro-2-methylindazol-6-yl)-2-methyl-indazole-7-carboxamide (Compound 300, 21.2 mg, 45.35 μmol, 47% yield) as a solid. LCMS (ES, m/z): 454 [M+H]⁺. ¹H NMR (400 MHz, DMSO-d₆) δ 11.02 (s, 1H), 8.74 (s, 1H), 8.46 (s, 1H), 8.02 (s, 1H), 7.11 (dd, J=12.2, 1.4 Hz, 1H), 6.23 (d, J=15.3 Hz, 1H), 4.21 (s, 3H), 4.16 (s, 3H), 3.88 (d, J=12.9 Hz, 2H), 3.14-2.86 (m, 2H), 2.80-2.66 (m, 1H), 2.62 (q, J=7.1 Hz, 2H), 2.05-1.90 (m, 2H), 1.43 (q, J=9.9 Hz, 2H), 1.05 (t, J=7.1 Hz, 3H). 19F NMR (376 MHz, DMSO-d₆) δ −109.48, −116.83.

### Example 103: Synthesis of Compound 301

### Synthesis of B93

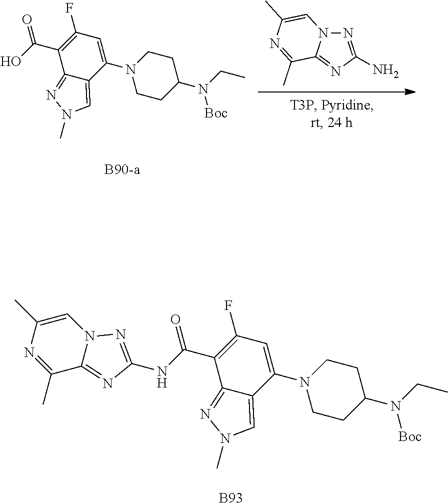

To a stirred mixture of 4-[4-[tert-butoxycarbonyl(ethyl)amino]-1-piperidyl]-6-fluoro-2-methyl-indazole-7-carboxylic acid (0.15 g, 356.7 μmol) and 6,8-dimethyl-[1,2,4]triazolo[1,5-a]-pyrazin-2-amine (75 mg, 463.8 μmol) in Pyridine (5 mL) was added T₃P (5 mL), and the reaction was stirred for 24 h at room temperature. The resulting mixture was diluted with water (30 mL) and extracted with EtOAc (3×10 mL). The combined organic layers were washed with brine (10 mL), dried over anhydrous Na₂SO₄, and filtered. The filtrate was concentrated under reduced pressure and purified by silica gel column chromatography (9% MeOH in DCM) to afford tert-butyl N-[1-[7-[(6,8-dimethyl-[1,2,4]triazolo[1,5-a]pyrazin-2-yl)carbamoyl]-6-fluoro-2-methyl-indazol-4-yl]-4-piperidyl]-N-ethyl-carbamate (B93, 0.12 g, 212.2 μmol, 59% yield) as a solid. LCMS (ES, m/z): 566 [M+H]⁺.

### Synthesis of Compound 301

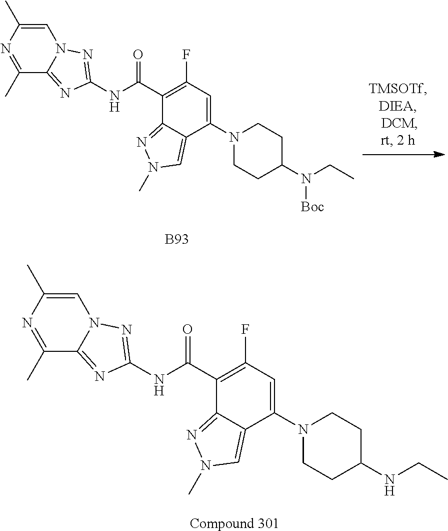

To a solution of tert-butyl N-[1-[7-[(6,8-dimethyl-[1,2,4]triazolo[1,5-a]pyrazin-2-yl)-carbamoyl]-6-fluoro-2-methyl-indazol-4-yl]-4-piperidyl]-N-ethyl-carbamate (0.13 g, 229.8 μmol) in DCM (3 mL) was added TMSOTf (153 mg, 689.5 μmol) and DIEA (148 mg, 1.15 mmol), and the reaction was stirred for 2 h at rt. The resulting mixture was concentrated under reduced pressure and purified by Prep-HPLC (Condition 2, Gradient 5) to afford N-(6,8-dimethyl-[1,2,4]triazolo[1,5-a]pyrazin-2-yl)-4-(4-(ethylamino)piperidin-1-yl)-6-fluoro-2-methyl-2H-indazole-7-carboxamide 2,2,2-trifluoroacetate (0.032 g, 55.2 μmol, 24% yield) as a solid. LCMS (ES, m/z): 466 [M+H]⁺. ¹H NMR (300 MHz, DMSO-d₆) δ 8.75 (s, 1H), 8.59 (s, 1H), 6.31 (d, J=15.7 Hz, 1H), 4.19 (s, 3H), 4.06 (d, J=13.0 Hz, 2H), 3.34 (s, 1H), 3.17-2.94 (m, 4H), 2.72 (s, 3H), 2.46 (s, 3H), 2.12 (d, J=12.4 Hz, 2H), 1.67 (d, J=11.2 Hz, 2H), 1.20 (t, J=7.2 Hz, 3H).

An analogous method was followed to obtain the following compounds.

| | Starting | |
|---|---|---|
| Compound | Material | Characterization |
| 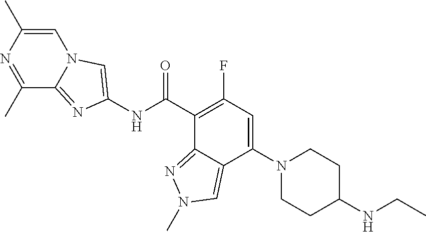 [SMILES: CCNC1CCN(c2cc(F)c(C(=O)Nc3cn4cc(C)nc(C)c4n3)c3nn(C)cc23)CC1] | 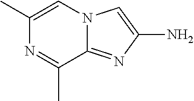 | Modification: Step 1 was ran at 80° C. for 8 h. The residue was purified by Prep-HPLC (Condition 2, Gradient 5) to afford N- (6,8-dimethylimidazo[1,2-a]pyrazin-2- yl)-4-(4-(ethylamino)-piperidin-1-y1)-6- fluoro-2-methyl-2H-indazole-7- carboxamide 2,2,2-trifluoroacetate (Compound 306, 0.018 g, 31.1 μmol, 20% yield) as a solid. LCMS (ES, m/z): 465 [M + H]⁺. 1H NMR (300 MHz, DMSO-d⁶) δ 11.59 (s, 1H), 8.78 (s, 1H), 8.43 (d, J = 13.2 Hz, 2H), 8.28 (s, 1H), 6.33 (d, J = 15.7 Hz, 1H), 4.23 (s, 3H), |
| | | 4.08 (d, J = 12.9 Hz, 2H), 3.35 (s, 1H), |
| Compound 306 | | 3.18-2.95 (m, 4H), 2.76 (s, 3H), 2.41 (s, |
| | | 3H), 2.12 (d, J = 11.0 Hz, 2H), 1.64 (d, |
| | | J = 10.4 Hz, 2H), 1.21 (t, J = 7.2 Hz, 3H). |
| 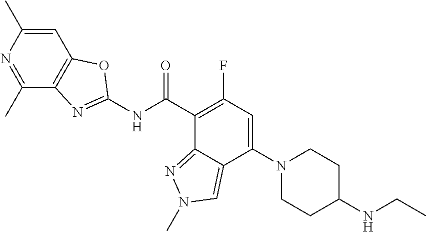 [SMILES: CCNC1CCN(c2cc(F)c(C(=O)Nc3nc4c(C)nc(C)cc4o3)c3nn(C)cc23)CC1] | 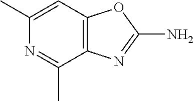 | Modification: Step 2 was performed using TFA in DCM for 1 h at room temperature. The resulting mixture was purified by reversed-phase flash chromatography (Condition 4, Gradient 2) to afford N- (4,6-dimethyl-oxazolo-[4,5-c]-pyridin-2- y1)-4-[4-(ethylamino)-1-piperidy1]-6- fluoro-2-methyl-indazole-7-carboxamide (Compound 380, 26 mg, 55.9 μmol, 45% yield) as a solid. LCMS (ES, m/z): 466 [M + H]⁺. ¹H NMR (300 MHz, DMSO-d₆) δ 12.48 (s, 1H), 8.93 (s, 1H), 8.50 (s, 2H), 7.84 (s, 1H), 6.39 (d, J = 15.9 Hz, 1H), 4.21 (s, 3H), 4.13 (d, J = 13.3 Hz, 2H), |
| | | 3.38 (s, 1H), 3.15 (t, J = 12.7 Hz, 2H), |
| Compound 380 | C133 | 3.05-3.03 (m, 2H), 2.74 (s, 3H), 2.66 (s, |
| | | 3H), 2.12 (d, J = 11.9 Hz, 2H), 1.63 (q, |
| | | J = 11.6 Hz, 2H), 1.20 (t, J = 7.2 Hz, 3H). |

An analogous method was followed, using B98 in place of B90, to obtain the following compounds.

| | Starting | |
|---|---|---|
| Compound | Material | Characterization |
| 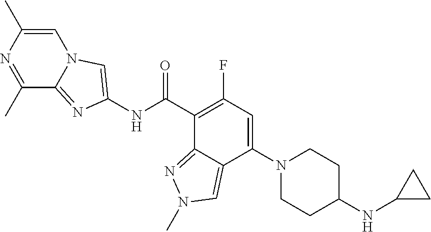 [SMILES: Cc1cn2cc(NC(=O)c3c(F)cc(N4CCC(NC5CC5)CC4)c4cn(C)nc34)nc2c(C)n1] | 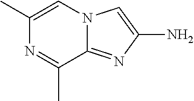 | Modification: Step 1 was ran at 80° C. for 5 h. The residue was purified by Prep- HPLC (Condition 2, Gradient 4) to afford 4-(4-(cyclopropylamino)- piperidin-1-yl)-N-(6,8-dimethyl- imidazo[1,2-a]pyrazin-2-y1)-6-fluoro- 2-methyl-2H-indazole-7-carboxamide bis(2,2,2-trifluoroacetate) (Compound 334, 0.025 g, 35.5 μmol, 29% yield) as a solid. LCMS (ES, m/z): 477 [M + H]⁺. ¹H NMR (300 MHz, Methanol-d₄) δ 8.62 (s, 1H), 8.59 (s, 1H), 8.44 (s, 1H), 6.39 (d, J = 15.7 Hz, 1H), 4.34 (s, 3H), |
| | | 4.21 (d, J = 13.2 Hz, 2H), 3.68-3.54 (m, |
| Compound 334 | | 1H), 3.21 (t, J = 12.7 Hz, 2H), 2.93 (s, |
| | | 3H), 2.91-2.80 (m, 1H), 2.55 (d, J = 1.0 |
| | | Hz, 3H), 2.36 (d, J = 12.6 Hz, 2H), |
| | | 1.88-1.77 (m, 2H), 1.01 (dd, J = 7.2, 5.6 |
| | | Hz, 2H), 0.93 (dd, J = 5.5, 3.7 Hz, 2H). |

### Example 104: Synthesis of C59

### Synthesis of C56

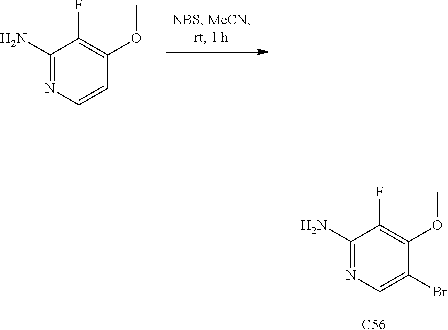

To a stirred solution of 3-fluoro-4-methoxy-pyridin-2-amine (31.4 g, 220.9 mmol) in MeCN (400 mL) was added NBS (39.3 g, 220.9 mmol) in portions at 0° C. The resulting mixture was stirred for 1 h at room temperature. The reaction was quenched by the addition of water (1000 mL) at room temperature. The resulting mixture was extracted with EtOAc (3×300 mL). The combined organic layers were washed with brine (3×400 mL), dried over anhydrous Na₂SO₄, and filtered. The filtrate was concentrated under reduced pressure and purified by trituration with n-Hexane (100 mL). The precipitated solids were collected by filtration and washed with n-Hexane (3×100 mL), to afford 5-bromo-3-fluoro-4-methoxy-pyridin-2-amine (C56, 48 g, 217.2 mmol, 98% yield) as a solid. LCMS (ES, m/z): 221 [M+H]⁺.

### Synthesis of C57

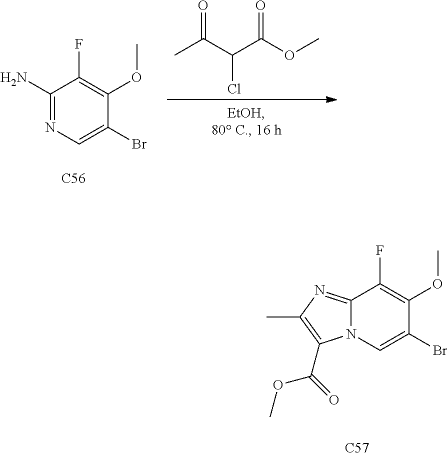

To a stirred solution of 5-bromo-3-fluoro-4-methoxypyridin-2-amine (1 g, 4.55 mmol) and methyl 2-chloro-3-oxobutanoate 1 g, 6.82 mmol) in EtOH (10 mL) at room temperature under N₂. The resulting mixture was stirred for 16 h at 80° C. under N₂. The mixture was allowed to cool down to room temperature. The reaction was concentrated under reduced pressure and purified by reversed-phase flash chromatography (Condition 3, Gradient 17) to afford methyl 6-bromo-8-fluoro-7-methoxy-2-methylimidazo[1,2-a]pyridine-3-carboxylate (268 mg, 19% yield) as a solid. LCMS (ES, m/z): 317 [M+H].

### Synthesis of C58

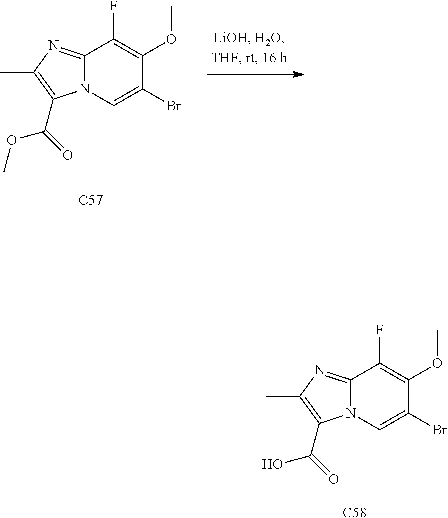

A solution of methyl 6-bromo-8-fluoro-7-methoxy-2-methyl-imidazo[1,2-a]pyridine-3-carboxylate (258 mg, 813.6 μmol) and LiOH·H₂O (341 mg, 8.14 mmol) in THF (4 mL) and H₂O (2 mL) was stirred for 16 h at room temperature. The resulting mixture was concentrated under reduced pressure. The resulting mixture was diluted with water (20 mL). The mixture was acidified to pH 6 with citric acid. The resulting mixture was extracted with EtOAc (3×10 mL). The combined organic layers were washed with brine (20 mL), dried over anhydrous Na₂SO₄, and filtered. The filtrate was concentrated under reduced pressure, to afford 6-bromo-8-fluoro-7-methoxy-2-methyl-imidazo[1,2-a]pyridine-3-carboxylic acid (C58, 180 mg, 593.9 μmol, 73% yield) as a solid. LCMS (ES, m/z): 303 [M+H]⁺.

### Synthesis of C59

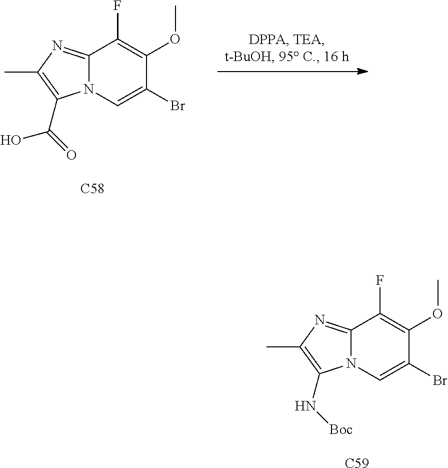 [SMILES: COc1c(Br)cn2c(C(=O)O)c(C)nc2c1F ; "C58" ; COc1c(Br)cn2c(NC(=O)OC(C)(C)C)c(C)nc2c1F ; "C59"]

To a stirred solution of 6-bromo-8-fluoro-7-methoxy-2-methyl-imidazo[1,2-a]pyridine-3-carboxylic acid (163 mg, 537.8 μmol) and DPPA (178 mg, 645.4 μmol) in t-BuOH (3 mL) was added TEA (82 mg, 806.7 μmol) at room temperature under N₂. The resulting mixture was stirred for 2 h at 90° C. under N₂. The mixture was concentrated under reduced pressure and purified by silica gel column chromatography (33% THF in PE) to afford tert-butyl N-(6-bromo-8-fluoro-7-methoxy-2-methyl-imidazo[1,2-a]pyridin-3-yl)carbamate (120 mg, 320.7 μmol, 60% yield) as a solid. LCMS (ES, m/z): 374 [M+H]⁺.

### Example 105: Synthesis of Compound 310

### Synthesis of B95

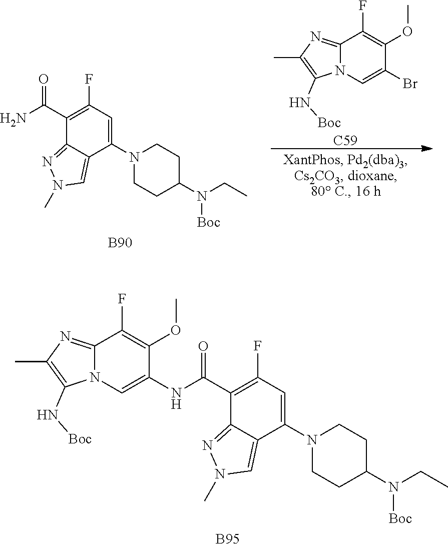

To a stirred solution of tert-butyl N-[1-(7-carbamoyl-6-fluoro-2-methyl-indazol-4-yl)-4-piperidyl]-N-ethyl-carbamate (140 mg, 333.7 μmol), Cs₂CO₃ (218 mg, 667.5 μmol) and tert-butyl N-(6-bromo-8-fluoro-7-methoxy-2-methyl-imidazo[1,2-a]pyridin-3-yl)carbamate (125 mg, 333.7 μmol) in dioxane (2 mL) were added XantPhos (39 mg, 66.75 μmol) and Pd₂(dba)₃ (31 mg, 33.37 μmol) at room temperature under N₂. The resulting mixture was stirred for 16 h at 80° C. under N₂. The mixture was concentrated under reduced pressure and purified by reversed-phase flash chromatography (Condition 3, Gradient 17) to afford tert-butyl N-[1-[7-[[3-(tert-butoxycarbonylamino)-8-fluoro-7-methoxy-2-methyl-imidazo[1,2-a]pyridin-6-yl]carbamoyl]-6-fluoro-2-methyl-indazol-4-yl]-4-piperidyl]-N-ethyl-carbamate (80 mg, 112.2 μmol, 34% yield) as a solid. LCMS (ES, m/z): 713 [M+H]⁺

### Synthesis of Compound 310

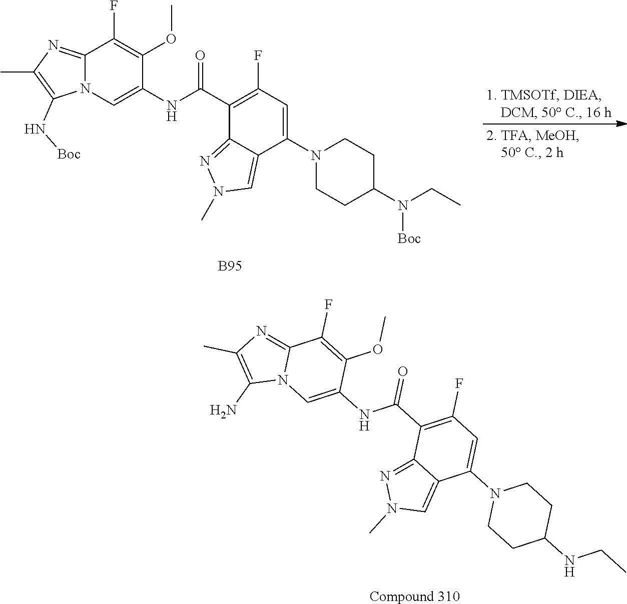

To a stirred solution of tert-butyl N-[1-[7-[[3-(tert-butoxycarbonylamino)-8-fluoro-7-methoxy-2-methyl-imidazo[1,2-a]pyridin-6-yl]carbamoyl]-6-fluoro-2-methyl-indazol-4-yl]-4-piperidyl]-N-ethyl-carbamate (60 mg, 84.18 μmol) and DIEA (54 mg, 420.9 μmol) in DCM (2 mL) was added TMSOTf (94 mg, 420.9 μmol) at room temperature under N₂. The resulting mixture was stirred for 16 h at 50° C. under N₂. The resulting mixture was concentrated under reduced pressure. The residue was diluted with MeOH (1 mL) and added TFA (0.2 mL), and the reaction was stirred for 2 h at 50° C. The resulting mixture was concentrated under reduced pressure and purified by reversed-phase flash chromatography (Condition 3, Gradient 3) to afford N-(3-amino-8-fluoro-7-methoxy-2-methyl-imidazo[1,2-a]pyridin-6-yl)-4-[4-(ethylamino)-1-piperidyl]-6-fluoro-2-methyl-indazole-7-carboxamide (Compound 310, 5.5 mg, 10.7 μmol, 13% yield) as a solid. LCMS (ES, m/z): 513 [M+H]⁺. ¹H NMR (300 MHz, Methanol-d₄) δ 9.16 (d, J=1.3 Hz, 1H), 8.47 (s, 1H), 6.23 (d, J=16.2 Hz, 1H), 4.29 (s, 3H), 4.25 (d, J=2.1 Hz, 3H), 4.04 (d, J=13.1 Hz, 2H), 3.10 (q, J=13.0, 11.2 Hz, 2H), 2.93 (d, J=10.3 Hz, 1H), 2.82 (q, J=7.2 Hz, 2H), 2.35 (s, 3H), 2.12 (d, J=12.6 Hz, 2H), 1.61 (q, J=10.6, 10.1 Hz, 2H), 1.41-1.25 (m, 1H), 1.22 (t, J=7.2 Hz, 3H).

### Example 106: Synthesis of B98

### Synthesis of B96

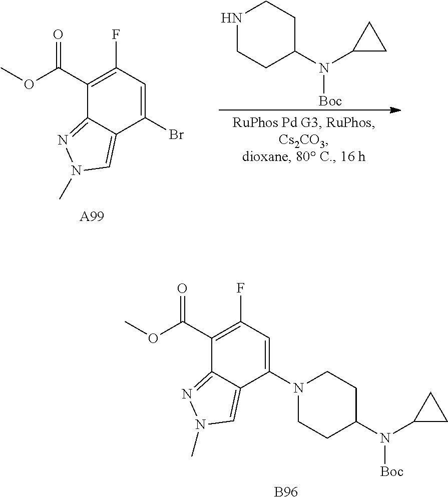

To a stirred mixture of methyl 4-bromo-6-fluoro-2-methyl-indazole-7-carboxylate (2 g, 6.97 mmol) in dioxane (25 mL) were added tert-butyl N-cyclopropyl-N-(4-piperidyl)carbamate (2.01 g, 8.36 mmol), Cs₂CO₃ (4.54 g, 13.93 mmol), Ruphos (650 mg, 1.39 mmol) and RuPhos Pd G3 (583 mg, 696.7 μmol), and the reaction was stirred for 16 h at 80° C. under N₂. The resulting mixture was diluted with water (80 mL) and extracted with DCM (2×80 mL). The combined organic layers were washed with water (2×100 mL), dried over anhydrous Na₂SO₄, and filtered. The filtrate was concentrated under reduced pressure and purified by silica gel column chromatography (20% EtOAc in DCM) to afford methyl 4-[4-[tert-butoxycarbonyl(cyclopropyl)amino]-1-piperidyl]-6-fluoro-2-methyl-indazole-7-carboxylate (1.05 g, 2.35 mmol, 34% yield) as an oil. LCMS (ES, m/z): 447 [M+H]⁺.

### Synthesis of B97

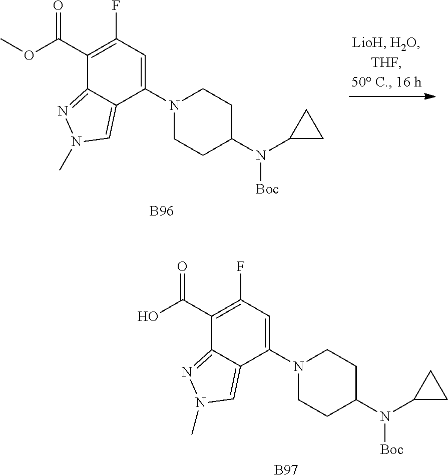

To a solution of methyl 4-[4-[tert-butoxycarbonyl(cyclopropyl)amino]-1-piperidyl]-6-fluoro-2-methyl-indazole-7-carboxylate (1 g, 2.24 mmol) in THF (10 mL), H₂O (5 mL) was added LiOH·H₂O (940 mg, 22.4 mmol), and the mixture was stirred for 16 h at 50° C. The resulting mixture was diluted with water and adjusted to pH 5-6 with 1 N HCl. The resulting mixture was extracted with DCM (2×50 mL). The combined organic layers were washed with water (2×80 mL), dried over anhydrous Na₂SO₄, and filtered. The filtrate was concentrated under reduced pressure, to afford 4-[4-[tert-butoxycarbonyl(cyclopropyl)amino]-1-piperidyl]-6-fluoro-2-methyl-indazole-7-carboxylic acid (820 mg, 1.90 mmol, 85% yield) as a solid. LCMS (ES, m/z): 433 [M+H]⁺.

### Synthesis of B98

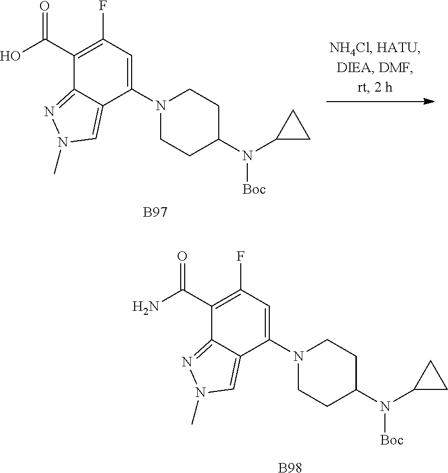 [SMILES: "B97" ; Cn1cc2c(N3CCC(N(C(=O)OC(C)(C)C)C4CC4)CC3)cc(F)c(C(=O)O)c2n1 ; "B98" ; Cn1cc2c(N3CCC(N(C(=O)OC(C)(C)C)C4CC4)CC3)cc(F)c(C(N)=O)c2n1]

To a solution of 4-[4-[tert-butoxycarbonyl(cyclopropyl)amino]-1-piperidyl]-6-fluoro-2-methyl-indazole-7-carboxylic acid (160 mg, 370 μmol) in DMF (2 mL) were added NH₄Cl (79 mg, 1.48 mmol), DIEA (191 mg, 1.48 mmol, 257 μL) and HATU (211 mg, 554.9 μmol), and the reaction was stirred for 2 h at rt. The reaction was diluted with water (20 mL) and extracted with EtOAc (2×20 mL). The combined organic layers were washed with water (2×30 mL), dried over anhydrous Na₂SO₄, and filtered. The filtrate was concentrated under reduced pressure and purified by silica gel column chromatography (5% MeOH in DCM) to afford tert-butyl N-[1-(7-carbamoyl-6-fluoro-2-methyl-indazol-4-yl)-4-piperidyl]-N-cyclopropylcarbamate (B98, 140 mg, 324.5 μmol, 88% yield) as a solid. LCMS (ES, m/z): 432 [M+H]⁺.

An analogous method for this step was followed to obtain the following compounds.

| Compound | Starting Material | Characterization |
|---|---|---|
| 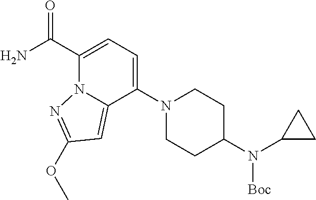 [SMILES: COc1cc2c(N3CCC(N(C(=O)OC(C)(C)C)C4CC4)CC3)ccc(C(N)=O)n2n1] | 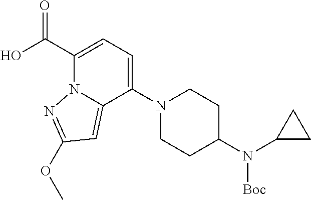 [SMILES: COc1cc2c(N3CCC(N(C(=O)OC(C)(C)C)C4CC4)CC3)ccc(C(=O)O)n2n1] | The residue was purified by silica gel column chromatography (100% EtOAc) to afford tert-butyl N-[1-(7- carbamoyl-2-methoxy-pyrazolo[1,5- alpyridin-4-y1)-4-piperidy1]-N- cyclopropyl-carbamate (D48, 80 mg, 186.3 μmol). LCMS (ES, m/z): 430 [M + H]⁺. |
| D48 | | |
| 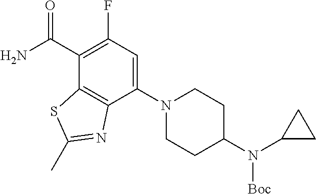 [SMILES: Cc1nc2c(N3CCC(N(C(=O)OC(C)(C)C)C4CC4)CC3)cc(F)c(C(N)=O)c2s1] | 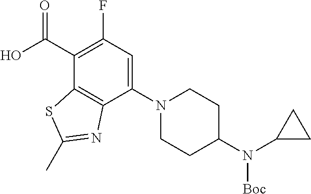 [SMILES: Cc1nc2c(N3CCC(N(C(=O)OC(C)(C)C)C4CC4)CC3)cc(F)c(C(=O)O)c2s1] | The residue was purified by silica gel column chromatography (67% EtOAc in PE) to afford tert-butyl N- [1-(7-carbamoyl-6-fluoro-2-methyl- 1,3-benzothiazol-4-yl)-4-piperidyl]- N-cyclopropyl-carbamate (D50, 75 mg, 167.2 μmol). LCMS (ES, m/z): 449 [M + H]⁻. |
| D50 | | |

### Example 107: Synthesis of Compound 320

### Synthesis of B99

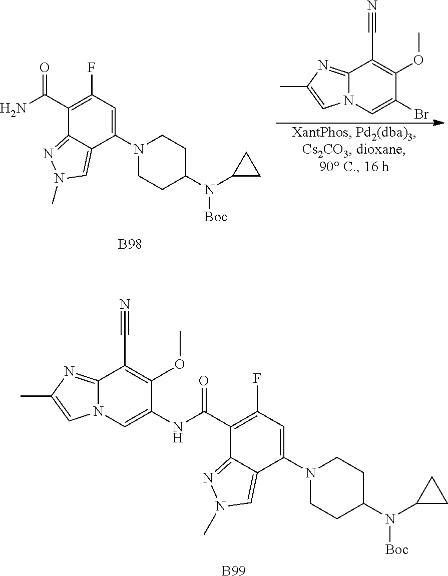

To a stirred mixture of tert-butyl N-[1-(7-carbamoyl-6-fluoro-2-methyl-indazol-4-yl)-4-piperidyl]-N-cyclopropyl-carbamate (120 mg, 278.1 μmol) in dioxane (1.5 mL) were added 6-bromo-7-methoxy-2-methyl-imidazo[1,2-a]pyridine-8-carbonitrile (89 mg, 333.7 μmol), Cs₂CO₃ (181 mg, 556.2 μmol), Xantphos (32 mg, 55.62 μmol) and Pd₂(dba)₃ (25 mg, 27.81 μmol), and the reaction was stirred for 3 h at 100° C. under N₂. The resulting mixture was diluted with H₂O (15 mL) and extracted with DCM (2×15 mL). The combined organic layers were washed with water (2×20 mL), dried over anhydrous Na₂SO₄, and filtered. The filtrate was concentrated under reduced pressure and purified by silica gel column chromatography (3% MeOH in DCM) to afford tert-butyl N-[1-[7-[(8-cyano-7-methoxy-2-methyl-imidazo[1,2-a]-pyridin-6-yl)carbamoyl]-6-fluoro-2-methyl-indazol-4-yl]-4-piperidyl]-N-cyclopropyl-carbamate (85 mg, 137.8 μmol, 50% yield) as a solid. LCMS (ES, m/z): 432 [M+H]⁺.

### Synthesis of Compound 320

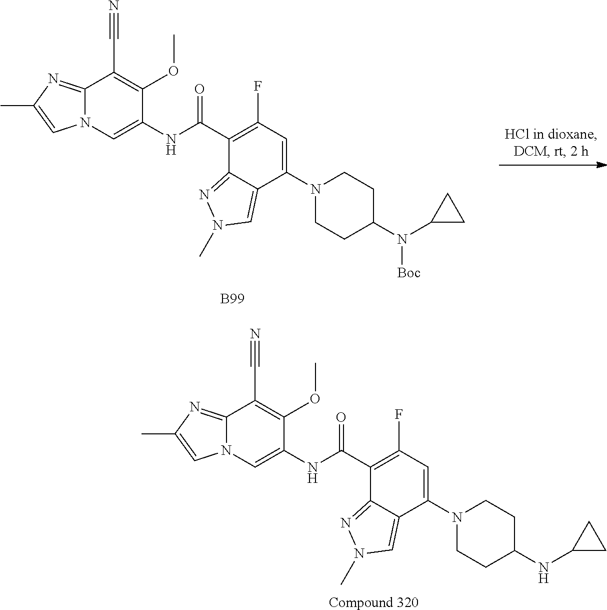

To a solution of tert-butyl N-[1-[7-[(8-cyano-7-methoxy-2-methyl-imidazo[1,2-a]pyridin-6-yl)carbamoyl]-6-fluoro-2-methyl-indazol-4-yl]-4-piperidyl]-N-cyclopropyl-carbamate (80 mg, 129.7 μmol) in DCM (1 mL) was added HCl (4.0 M in 1,4-dioxane, 300 μL), and the reaction was stirred for 2 h at rt. The resulting mixture was concentrated under reduced pressure and purified by Prep-HPLC (Condition 5, Gradient 8) to afford N-(8-cyano-7-methoxy-2-methyl-imidazo[1,2-a]pyridin-6-yl)-4-[4-(cyclopropylamino)-1-piperidyl]-6-fluoro-2-methyl-indazole-7-carboxamide (Compound 320, 20 mg, 38.7 μmol, 30% yield) as a solid. LCMS (ES, m/z): 538 [M+H]⁺. ¹H NMR (400 MHz, DMSO-d₆) δ 11.60 (s, 1H), 9.34 (s, 1H), 8.79 (s, 1H), 7.60 (d, J=1.4 Hz, 1H), 6.21 (d, J=16.2 Hz, 1H), 4.21 (s, 3H), 3.91 (d, J=13.1 Hz, 2H), 3.78 (s, 3H), 3.12 (t, J=11.4 Hz, 2H), 2.80 (p, J=5.3 Hz, 1H), 2.27 (d, J=1.3 Hz, 3H), 2.11 (tt, J=6.7, 3.6 Hz, 1H), 1.99 (d, J=12.6 Hz, 2H), 1.44 (q, J=11.3, 9.9 Hz, 2H), 0.39 (dt, J=6.2, 3.0 Hz, 2H), 0.27-0.19 (m, 2H).

An analogous method was followed to obtain the following compounds.

| | Starting | |
|---|---|---|
| Compound | Material | Characterization |
| 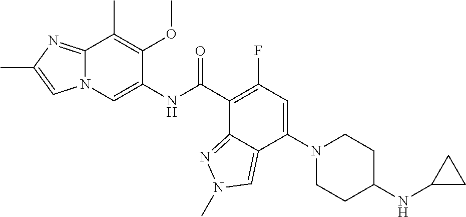 [SMILES: COc1c(NC(=O)c2c(F)cc(N3CCC(NC4CC4)CC3)c3cn(C)nc23)cn2cc(C)nc2c1C] | 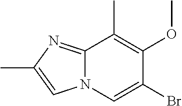 | Modification: Step 1 was ran for 5 h. Step 2 was ran using TFA (1 mL) and DCM (3 mL) at room temperature for 2 h. The residue was purified by reversed-phase flash chromatography (Condition 3, Gradient 1 afford 4-[4- (cyclopropylamino)-1-piperidy1]- 6-fluoro-N-(7-methoxy-2,8- dimethyl-imidazo[1,2-a]pyridin- 6-y1)-2-methyl-indazole-7- carboxamide (Compound 397, 26 |
| | | mg, 51.4 μmol, 42% yield) as a |
| Compound 397 | | solid. LCMS (ES, m/z): 506 |
| | | [M + H]⁺. ¹H NMR (400 MHz, |
| | | DMSO-d₆) δ 11.47 (s, 1H), 9.49 (s, |
| | | 1H), 8.83 (s, 1H), 7.65 (s, 1H), |
| | | 6.24 (d, J = 16.1 Hz, 1H), 4.25 (s, |
| | | 3H), 3.95 (s, 3H), 3.90 (s, 2H), |
| | | 3.14 (t, J = 11.8 Hz, 2H), 2.81 (s, |
| | | 1H), 2.44 (s, 3H), 2.30 (s, 4H), |
| | | 2.15-2.09 (m, 1H), 2.04-1.95 (m, |
| | | 2H), 1.45 (q, J = 11.0, 10.6 Hz, |
| | | 2H), 0.40 (dd, J = 6.6, 2.2 Hz, 2H), |
| | | 0.24 (q, J = 3.4, 2.9 Hz, 2H). |
| 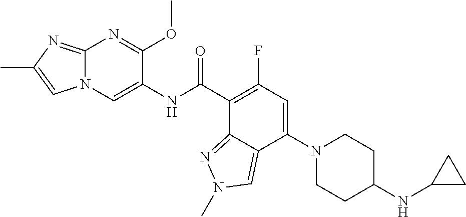 [SMILES: COc1nc2nc(C)cn2cc1NC(=O)c1c(F)cc(N2CCC(NC3CC3)CC2)c2cn(C)nc12] | 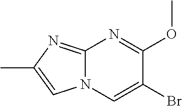 | Modification: Step 1 was ran for 5 h. Step 2 was ran using TFA (1 mL) and DCM (3 mL) at room temperature for 2 h. The residue was purified by reversed-phase flash chromatography (Condition 3, Gradient 11) to afford 4-[4- (cyclopropylamino)-1-piperidy1]- 6-fluoro-N-(7-methoxy-2-methyl- imidazo-[1,2-a]pyrimidin-6-yl)-2- methyl-indazole-7-carboxamide (Compound 398, 25 mg, 50.8 |
| | | μmol, 50% yield) as a solid. |
| Compound 398 | | LCMS (ES, m/z): 493 [M + H]⁺ ¹H |
| | | NMR (400 MHz, DMSO-d₆) δ |
| | | 11.54 (s, 1H), 9.41 (s, 1H), 8.80 (s, |
| | | 1H), 7.29 (d, J = 1.2 Hz, 1H), 6.21 |
| | | (d, J = 16.2 Hz, 1H), 4.20 (s, 3H), |
| | | 3.91 (d, J = 13.2 Hz, 2H), 3.60 (s, |
| | | 3H), 3.13 (t, J = 11.4 Hz, 2H), 2.80 |
| | | (s, 1H), 2.27 (s, 1H), 2.19 (d, J = |
| | | 1.1 Hz, 3H), 2.11 (tt, J = 6.7, 3.6 |
| | | Hz, 1H), 1.99 (d, J = 12.1 Hz, 2H), |
| | | 1.44 (q, J = 13.0, 11.3 Hz, 2H), |
| | | 0.40 (td, J = 6.3, 4.0 Hz, 2H), 0.27- |
| | | 0.19 (m, 2H). |
| 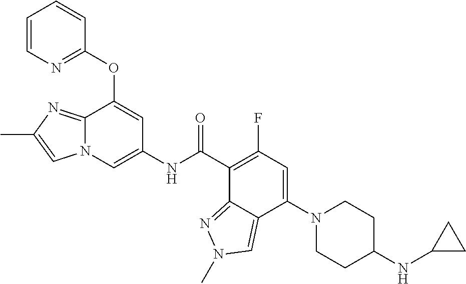 [SMILES: Cc1cn2cc(NC(=O)c3c(F)cc(N4CCC(NC5CC5)CC4)c4cn(C)nc34)cc(Oc3ccccn3)c2n1] | 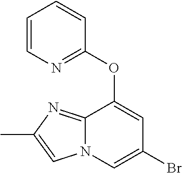 | Modification: Step 1 was ran for 5 h. Step 2 was ran using TFA (1 mL) and DCM (3 mL) at room temperature for 2 h. The crude product was purified by Prep-HPLC (Condition 6, Gradient 3) to afford 4-[4- (cyclopropylamino)-1-piperidyl]- 6-fluoro-2-methyl-N-[2-methyl-8- (2-pyridyloxy)-imidazo[1,2- alpyridin-6-yl]-indazole-7- carboxamide (Compound 399, 9.1 mg, 16.4 μmol, 13% yield) as a solid. LCMS (ES, m/z): 555 [M + H]⁺. ¹H NMR (400 MHz, DMSO-d₆) δ 10.88 (s, 1H), 9.26 (d, J = 1.8 Hz, 1H), 8.73 (s, 1H), |
| | | 8.10 (dd, J = 4.9, 2.0 Hz, 1H), |
| Compound 399 | | 7.94-7.85 (m, 1H), 7.81 (d, J = 1.0 |
| | | Hz, 1H), 7.20-7.11 (m, 3H), 6.23 |
| | | (d, J = 15.3 Hz, 1H), 4.17 (s, 3H), |
| | | 3.86 (d, J = 12.9 Hz, 2H), 3.07 (t, |
| | | J = 11.9 Hz, 2H), 2.83-2.73 (m, |
| | | 1H), 2.40-2.30 (m, 1H), 2.23 (s, |
| | | 3H), 2.11 (tt, J = 6.7, 3.6 Hz, 1H), |
| | | 1.98 (d, J = 12.2 Hz, 2H), 1.46- |
| | | 1.42 (m, 1H), 0.40 (td, J = 6.3, 4.0 |
| | | Hz, 2H), 0.27-0.19 (m, 2H). |
| 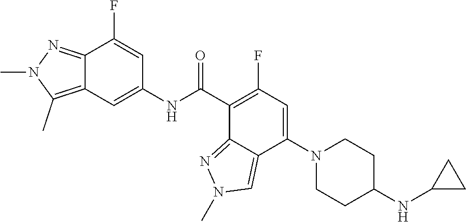 [SMILES: Cc1c2cc(NC(=O)c3c(F)cc(N4CCC(NC5CC5)CC4)c4cn(C)nc34)cc(F)c2nn1C] | 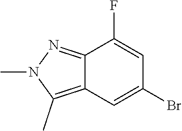 | Modification: Step 1 was ran for 5 h The crude product was purified by Prep-HPLC (Condition 5, Gradient 4) to afford 4-[4- (cyclopropyl-amino)-1-piperidyl]- 6-fluoro-N-(7-fluoro-2,3- dimethyl-indazol-5-y1)-2-methyl- indazole-7-carboxamide (Compound 403, 30 mg, 60.8 μmol, 36% yield) as a solid. LCMS (ES, m/z): 494 [M + H]⁺. ¹H NMR (400 MHz, DMSO-d₆) δ 10.88 (s, 1H), 8.71 (s, 1H), 7.98 (d, J = 1.6 Hz, 1H), 7.25 (dd, J = 13.3, |
| | | 1.6 Hz, 1H), 6.20 (d, J = 15.2 Hz, |
| Compound 403 | | 1H), 4.18 (s, 3H), 4.06 (s, 3H), |
| | | 3.83 (d, J = 12.8 Hz, 2H), 3.04 (t, |
| | | J = 11.4 Hz, 2H), 2.77 (dd, J = 9.3, |
| | | 5.0 Hz, 1H), 2.58 (s, 3H), 2.10 (tt, |
| | | J = 6.7, 3.6 Hz, 1H), 2.00-1.92 (m, |
| | | 2H), 1.50-1.36 (m, 2H), 0.37 (dt, |
| | | J = 6.2, 3.0 Hz, 2H), 0.25-0.17 (m, |
| | | 2H). |
| 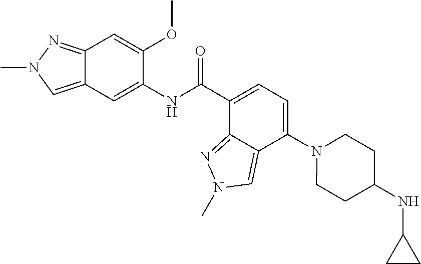 | 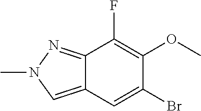 | Modification: Step 2 was ran using TFA in DCM for 2 h at room temperature. The crude product was purified by Prep-HPLC (Condition 5, Gradient 16) to afford 4-[4- (cyclopropylamino)-1-piperidyl]- 6-fluoro-N-(7-fluoro-6-methoxy- 2-methyl-indazol-5-yl)-2-methyl- indazole-7-carboxamide (Compound 405, 24.6 mg, 48.3 μmol, 33% yield) as a solid. LCMS (ES, m/z): 510 [M + H]⁻. ¹H NMR (400 MHz, DMSO-d₆) δ 11.55 (s, 1H), 8.81 (s, 1H), 8.63 (s, 1H), 8.37 (d, J = 2.8 Hz, 1H), 6.23 |
| | | (d, J = 16.1 Hz, 1H), 4.25 (s, 3H), |
| Compound 405 | | 4.15 (s, 3H), 4.11 (d, J = 1.1 Hz, |
| | | 3H), 3.91 (d, J = 13.1 Hz, 2H), |
| | | 3.18-2.99 (m, 2H), 2.80 (dt, J = |
| | | 9.9, 5.6 Hz, 1H), 2.32 (d, J = 14.8 |
| | | Hz, 1H), 2.11 (tq, J = 8.5, 4.8, 4.2 |
| | | Hz, 1H), 1.99 (dd, J = 13.0, 3.9 Hz, |
| | | 2H), 1.56-1.35 (m, 2H), 0.40 (td, |
| | | J = 6.3, 4.0 Hz, 2H), 0.29-0.18 (m, |
| | | 2H). |
| 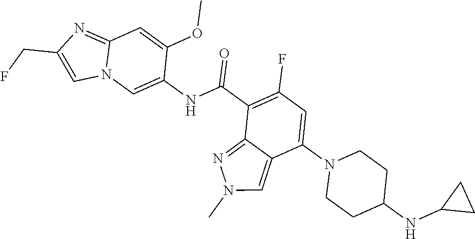 [SMILES: COc1cc2nc(CF)cn2cc1NC(=O)c1c(F)cc(N2CCC(NC3CC3)CC2)c2cn(C)nc12] | 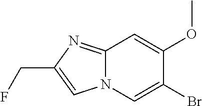 | Modification: Step 1 was ran for 4 h. Step 2 was ran using TFA in DCM for 2 h at room temperature. The residue was purified by Prep- HPLC (Condition 2, Gradient 4) to afford [cyclopropyl-[1-[6-fluoro- 7-[[2-(fluoromethyl)-7-methoxy- imidazo[1,2-a]-pyridin-6- yl]carbamoyl]-2-methyl-indazol- 4-y1]-4-piperidyl]amino] 2,2,2- trifluoroacetate (Compound 407, 16.1 mg, 25.9 μmol, 14% yield) as a solid. LCMS (ES, m/z): 510 [M + H]⁺. ¹H NMR (400 MHz, DMSO-d₆) δ 11.90 (s, 1H), 9.96 (s, |
| | | 1H), 8.93 (s, 1H), 8.92-8.90 (m, |
| Compound 407 | C117 | 2H), 8.36 (d, J = 4.3 Hz, 1H), 7.36 |
| | | (s, 1H), 6.36 (d, J = 15.9 Hz, 1H), |
| | | 5.67 (d, J = 47.6 Hz, 2H), 4.27 (s, |
| | | 3H), 4.24 (s, 3H), 4.13 (d, J = 13.3 |
| | | Hz, 2H), 3.52 (s, 1H), 3.15 (t, J = |
| | | 12.6 Hz, 2H), 2.79 (s, 1H), 2.21 (d, |
| | | J = 12.4 Hz, 2H), 1.73 (td, J = |
| | | 14.3, 12.8, 6.8 Hz, 2H), 0.87-0.80 |
| | | (m, 4H). |
| 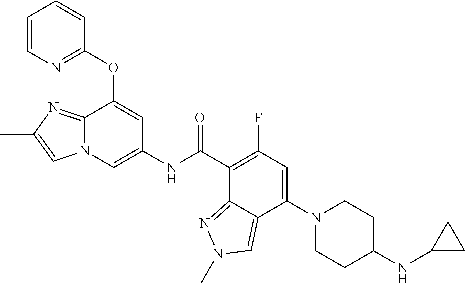 [SMILES: Cc1cn2cc(NC(=O)c3c(F)cc(N4CCC(NC5CC5)CC4)c4cn(C)nc34)cc(Oc3ccccn3)c2n1] | 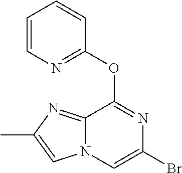 | Modification: Step 1 was ran for 5 h. Step 2 was ran using TFA (1 mL) and DCM (3 mL) at room temperature for 2 h. The crude product was purified by Prep-HPLC (Condition 6, Gradient 5) to afford 4-[4- (cyclopropylamino)-1-piperidy1]- 6-fluoro-2-methyl-N-[2-methyl-8- (2-pyridyloxy)imidazo[1,2-a]- pyrazin-6-yl]indazole-7- carboxamide (Compound 408, 34.9 mg, 62.8 μmol, 59% yield) as a solid. LCMS (ES, m/z): 556 [M + H]⁺. ¹H NMR (400 MHz, DMSO-d₆) δ 11.28 (s, 1H), 9.19 (s, 1H), 8.77 (s, 1H), 8.33 (ddd, J = |
| | | 4.9, 2.0, 0.8 Hz, 1H), 8.07-7.98 |
| Compound 408 | C118 | (m, 2H), 7.43-7.31 (m, 2H), 6.21 |
| | | (d, J = 16.1 Hz, 1H), 4.02 (s, 3H), |
| | | 3.90 (d, J = 13.2 Hz, 2H), 3.12 (t, |
| | | J = 11.5 Hz, 2H), 2.80 (dt, J = 9.7, |
| | | 5.4 Hz, 1H), 2.36 (d, J = 0.8 Hz, |
| | | 3H), 2.11 (ddt, J = 8.4, 5.2, 2.6 Hz, |
| | | 1H), 1.98 (d, J = 12.0 Hz, 2H), |
| | | 1.43 (q, J = 11.3, 9.6 Hz, 2H), |
| | | 0.44-0.35 (m, 2H), 0.30-0.19 (m, |
| | | 2H). |
| 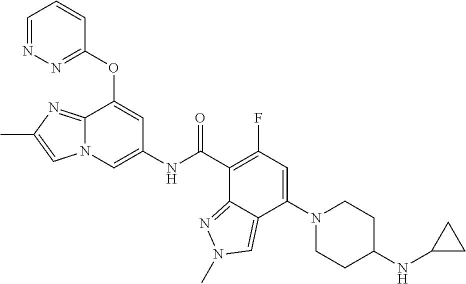 [SMILES: Cc1cn2cc(NC(=O)c3c(F)cc(N4CCC(NC5CC5)CC4)c4cn(C)nc34)cc(Oc3cccnn3)c2n1] | 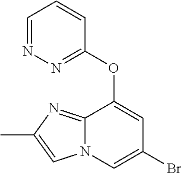 | Modification: Step 1 was ran for 5 h. Step 2 was ran using TFA in DCM at room temperature for 2 h. The resulting mixture was purified by reversed-phase flash chromatography (Condition 3, Gradient 23) to afford 4-[4- (cyclopropylamino)-1-piperidyl]- 6-fluoro-2-methyl-N-(2-methyl-8- pyridazin-3-yloxy-imidazo[1,2- alpyridin-6-yl)-indazole-7- carboxamide (Compound 409, 0.32 g, 576 μmol, 291% yield) as a solid. LCMS (ES, m/z): 556 [M + H]⁺. ¹H NMR (400 MHz, DMSO-d₆) δ 10.94 (s, 1H), 9.31 (d, J = 1.7 Hz, 1H), 9.03 (dd, J = 4.6, |
| | | 1.3 Hz, 1H), 8.74 (s, 1H), 7.87 - |
| Compound 409 | C119 | 7.78 (m, 2H), 7.62 (dd, J = 9.0, 1.3 |
| | | Hz, 1H), 7.29 (d, J = 1.8 Hz, 1H), |
| | | 6.23 (d, J = 15.3 Hz, 1H), 4.18 (s, |
| | | 3H), 3.87 (d, J = 13.1 Hz, 2H), |
| | | 3.13-3.02 (m, 2H), 2.24 (s, 3H), |
| | | 2.12 (tt, J = 6.8, 3.6 Hz, 1H), 1.99 |
| | | (d, J = 12.3 Hz, 2H), 1.45 (q, J = |
| | | 10.0 Hz, 2H), 0.40 (dt, J = 6.2, 3.0 |
| | | Hz, 2H), 0.27-0.19 (m, 2H) |
| 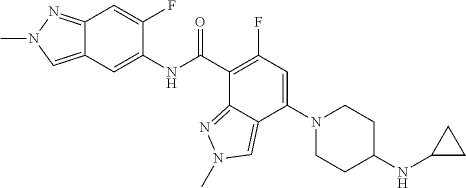 [SMILES: Cn1cc2cc(NC(=O)c3c(F)cc(N4CCC(NC5CC5)CC4)c4cn(C)nc34)c(F)cc2n1] | 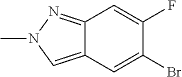 | Modification: Step 1 was ran for 2 h. The crude product was purified by Prep-HPLC (Condition 5, Gradient 10a) to afford 4-[4- (cyclopropylamino)-1-piperidy1]- 6-fluoro-N-(6-fluoro-2-methyl- indazol-5-yl)-2-methyl-indazole- 7-carboxamide (Compound 422, 30 mg, 62.6 μmol, 36% yield) as a solid. LCMS (ES, m/z): 480 M + H]⁺. ¹H NMR (400 MHz, DMSO-d₆) δ 11.46 (d, J = 3.3 Hz, |
| | | 1H), 8.81 (s, 1H), 8.75 (d, J = 7.8 |
| Compound 422 | C120 | Hz, 1H), 8.35 (s, 1H), 7.50 (d, J = |
| | | 12.1 Hz, 1H), 6.23 (d, J = 16.1 Hz, |
| | | 1H), 4.19 (s, 3H), 4.14 (s, 3H), |
| | | 3.91 (d, J = 13.0 Hz, 2H), 3.18- |
| | | 3.07 (m, 2H), 2.80 (p, J = 5.4 Hz, |
| | | 1H), 2.30 (s, 1H), 2.11 (ddd, J = |
| | | 14.2, 7.2, 3.8 Hz, 1H), 1.99 (dd, |
| | | J = 13.1, 4.0 Hz, 2H), 1.52-1.38 (m, |
| | | 2H), 0.40 (td, J = 6.3, 4.0 Hz, 2H), |
| | | 0.27-0.19 (m, 2H). |
| 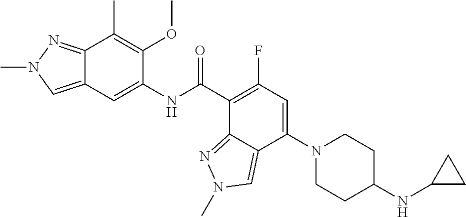 [SMILES: COc1c(NC(=O)c2c(F)cc(N3CCC(NC4CC4)CC3)c3cn(C)nc23)cc2cn(C)nc2c1C] | 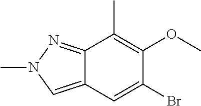 | Modification: Step 1 was ran for 5 h. Step 2 was ran using TFA in DCM at room temperature for 2 h. The crude product was purified by Prep-HPLC (Condition 6, Gradient 5) to afford 4-[4- (cyclopropylamino)-1-piperidy1]- 6-fluoro-N-(6-methoxy-2,7- dimethyl-indazol-5-y1)-2-methyl- indazole-7-carboxamide (Compound 425, 33.7 mg, 66.7 μmol, 58% yield) as a solid. LCMS (ES, m/z): 506 [M + H]⁺. ¹H |
| | | NMR (400 MHz, DMSO-d₆) δ |
| | | 11.47 (s, 1H), 8.80 (s, 1H), 8.67 (s, |
| Compound 425 | C122 | 1H), 8.22 (s, 1H), 6.22 (d, J = 16.1 |
| | | Hz, 1H), 4.25 (s, 3H), 4.13 (s, 3H), |
| | | 3.90 (s, 3H), 3.90 (d, J = 12.9 Hz, |
| | | 2H), 3.16-3.05 (m, 2H), 2.80 (dt, |
| | | J = 9.9, 5.6 Hz, 1H), 2.48 (s, 3H), |
| | | 2.31-2.25 (m, 1H), 2.12 (dd, J = |
| | | 6.8, 3.6 Hz, 1H), 1.99 (d, J = 12.4 |
| | | Hz, 2H), 1.47-1.44 (m, 2H), 0.40 |
| | | (dt, J = 6.2, 3.0 Hz, 2H), 0.27-0.20 |
| | | (m, 2H). |
| 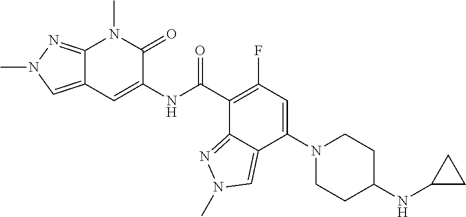 [SMILES: Cn1cc2c(N3CCC(NC4CC4)CC3)cc(F)c(C(=O)Nc3cc4cn(C)nc4n(C)c3=O)c2n1] | 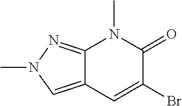 | Modification: Step 1 was ran at 100° C. for 3 h. The crude product was purified by Prep-HPLC (Condition 5, Gradient 12) to afford 4-[4- (cyclopropylamino)-1-piperidy1]- N-(2,7-dimethyl-6-oxo- pyrazolo[3,4-b]pyridin-5-y1)-6- fluoro-2-methyl-indazole-7- carboxamide (Compound 426, 40 mg, 81.2 μmol, 60% yield) as a solid. LCMS (ES, m/z): 493 [M + H]⁺. ¹H NMR (400 MHz, |
| | | DMSO-d₆) δ 11.57 (s, 1H), 8.75 |
| Compound 426 | C125 | (d, J = 4.1 Hz, 2H), 8.07 (s, 1H), |
| | | 6.18 (d, J = 16.1 Hz, 1H), 4.18 (s, |
| | | 3H), 3.93 (s, 3H), 3.91-3.83 (m, |
| | | 2H), 3.57 (s, 3H), 3.14-3.02 (m, |
| | | 2H), 2.77 (dt, J = 9.9, 5.7 Hz, 1H), |
| | | 2.10 (tt, J = 6.8, 3.6 Hz, 1H), 1.96 |
| | | (dd, J = 12.7, 3.9 Hz, 2H), 1.49- |
| | | 1.35 (m, 2H), 0.37 (dt, J = 6.2, 3.0 |
| | | Hz, 2H), 0.25-0.17 (m, 2H). |
| 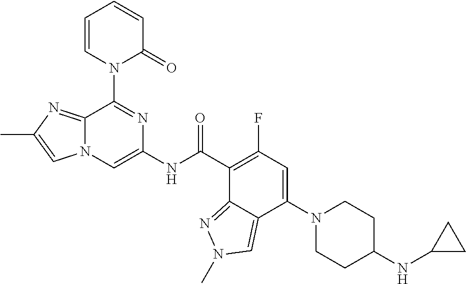 [SMILES: Cc1cn2cc(NC(=O)c3c(F)cc(N4CCC(NC5CC5)CC4)c4cn(C)nc34)nc(-n3ccccc3=O)c2n1] | 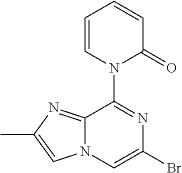 | Modificaiton: Step 1 was ran for 5 h. Step 2 was ran using TFA in DCM for 2 h at room temperature. The crude product was purified by Prep-HPLC (Condition 5, Gradient 11) to afford 4-[4- (cyclopropylamino)-1-piperidyl]- 6-fluoro-2-methyl-N-[2-methyl-8- (2-oxo-1-pyridyl)-imidazo[1,2-a]- pyrazin-6-yl]-indazole-7- carboxamide (Compound 427, 28.8 mg, 51.8 μmol, 38% yield) as a solid. LCMS (ES, m/z): 556 [M + H]⁺. ¹H NMR (400 MHz, DMSO-d₆) δ 11.38 (s, 1H), 9.57 (s, 1H), 8.80 (s, 1H), 8.17 (d, J = 0.9 Hz, 1H), 7.82 (dd, J = 6.9, 2.0 Hz, |
| | | 1H), 7.63 (ddd, J = 9.1, 6.6, 2.1 |
| Compound 427 | | Hz, 1H), 6.56 (dt, J = 9.4, 1.1 Hz, |
| | | 1H), 6.41 (td, J = 6.7, 1.2 Hz, 1H), |
| | | 6.24 (d, J = 16.1 Hz, 1H), 4.16 (s, |
| | | 3H), 3.91 (d, J = 12.9 Hz, 2H), |
| | | 3.19-3.08 (m, 2H), 2.80 (dt, J = |
| | | 9.7, 5.4 Hz, 1H), 2.37 (s, 3H), 2.30 |
| | | (s, 1H), 2.15-2.05 (m, 1H), 2.02- |
| | | 1.94 (m, 2H), 1.53-1.32 (m, 2H), |
| | | 0.44-0.35 (m, 2H), 0.26-0.18 (m, |
| | | 2H). |

### Example 108: Synthesis of C63

### Synthesis of C61

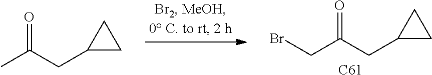

A solution of 1-cyclopropylpropan-2-one (1.5 g, 15.28 mmol) in MeOH (30 mL) was treated with Br₂ (1.95 g, 12.23 mmol) at 0° C., and the reaction was stirred for 2 h at room temperature under N₂. The resulting mixture was diluted with water (20 mL) and extracted with EtOAc (3×20 mL). The combined organic layers were washed with brine (40 mL), dried over anhydrous Na₂SO₄, and filtered. The filtrate was concentrated under reduced pressure and purified by silica gel column chromatography (33% EtOAc in PE) to afford 1-bromo-3-cyclopropyl-propan-2-one (1.2 g, 6.78 mmol, 44% yield) as an oil. ¹H NMR (300 MHz, CDCl₃) δ 3.97 (s, 2H), 2.57 (d, J=7.0 Hz, 2H), 1.06 (dqd, J=15.3, 7.4, 4.9 Hz, 1H), 0.71-0.58 (m, 2H), 0.19 (q, J=5.2 Hz, 2H).

### Synthesis of C62

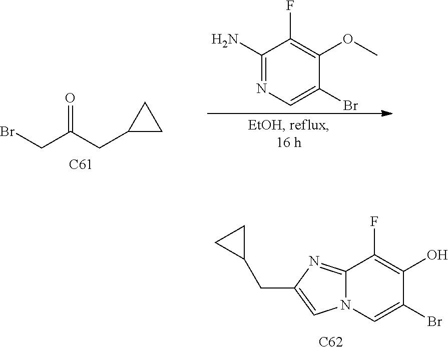 [SMILES: O=C(CBr)CC1CC1 ; COc1c(Br)cnc(N)c1F ; "C61" ; Oc1c(Br)cn2cc(CC3CC3)nc2c1F ; "C62"]

A solution of 5-bromo-3-fluoro-4-methoxy-pyridin-2-amine (1.5 g, 6.78 mmol) in EtOH (15 mL) was treated with 1-bromo-3-cyclopropyl-propan-2-one (1.2 g, 6.78 mmol) at rt under N₂. The resulting mixture was stirred for 16 h at 80° C. under N₂. The resulting mixture was concentrated off and diluted with water (20 mL). The mixture was neutralized to PH 7 and extracted with EtOAc (3×20 mL). The combined organic layers were washed with brine (40 mL), dried over anhydrous Na₂SO₄, and filtered. The filtrate was concentrated under reduced pressure and purified by silica gel column chromatography (50% EtOAc in PE) to afford 6-bromo-2-(cyclopropylmethyl)-8-fluoro-imidazo[1,2-a]pyridin-7-ol (1 g, 3.51 mmol, 52% yield) as a solid. LCMS (ES, m/z): 285 [M+H]⁺.

### Synthesis of C63

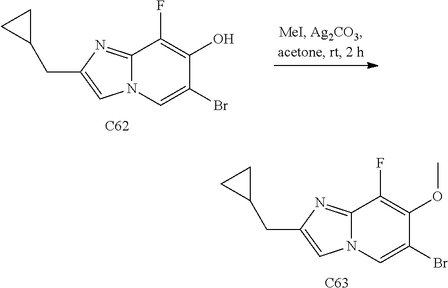 [SMILES: Oc1c(Br)cn2cc(CC3CC3)nc2c1F ; "C62" ; COc1c(Br)cn2cc(CC3CC3)nc2c1F ; "C63"]

A solution of 6-bromo-2-(cyclopropylmethyl)-8-fluoro-imidazo[1,2-a]pyridin-7-ol (1 g, 3.51 mmol) in Acetone (10 mL) was treated with Ag₂CO₃ (1.93 g, 7.01 mmol) and Mel (547 mg, 3.86 mmol), and the reaction was stirred for 2 h at room temperature under N₂. The resulting mixture was quenched by the addition of water (20 mL) and extracted with EtOAc (3×20 mL). The combined organic layers were washed with brine (30 mL), dried over anhydrous Na₂SO₄, and filtered. The filtrate was concentrated under reduced pressure, to afford 6-bromo-2-(cyclopropylmethyl)-8-fluoro-7-methoxy-imidazo[1,2-a]pyridine (0.4 g, 1.34 mmol, 38% yield) as a solid. LCMS (ES, m/z): 299 [M+H]⁺.

### Example 109: Synthesis of C64

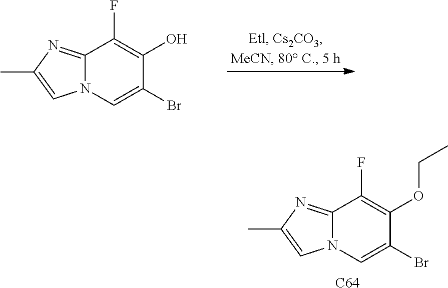

To a stirred mixture of 6-bromo-8-fluoro-2-methyl-imidazo[1,2-a]pyridin-7-ol (0.2 g, 816.2 μmol) and iodoethane (190 mg, 1.22 mmol, 98.42 μL) in MeCN (3 mL) were added Cs₂CO₃ (532 mg, 1.63 mmol), and the reaction was stirred for 5 h at 80° C. The resulting mixture was filtered, concentrated under reduced pressure and purified by silica gel column chromatography (25% EtOAc in PE) to afford 6-bromo-7-ethoxy-8-fluoro-2-methyl-imidazo-[1,2-a]pyridine (0.15 g, 512.6 μmol, 68% yield) as a solid. LCMS (ES, m/z): 273 [M+H]⁺.

### Example 110: Synthesis of C68

### Synthesis of C65

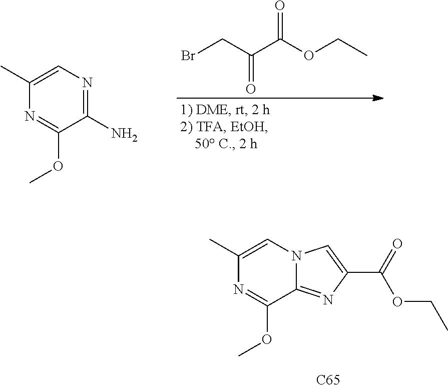

To a stirred mixture of 3-methoxy-5-methyl-pyrazin-2-amine (1.85 g, 13.29 mmol) and ethyl 3-bromo-2-oxo-propanoate (3.1 g, 15.95 mmol) in DME (40 mL). The resulting mixture was stirred for 2 h at room temperature. The precipitated solids were collected by filtration and dissolved in EtOH (40 mL). To the above mixture was added TFA (1.5 g, 13.29 mmol), and the reaction was stirred for 2 h at 50° C. The precipitated solids were collected by filtration and washed with EtOAc (3×10 mL) to afford ethyl 8-methoxy-6-methyl-imidazo[1,2-a]pyrazine-2-carboxylate (1 g, 4.25 mmol, 32% yield) as a solid.

### Synthesis of C66

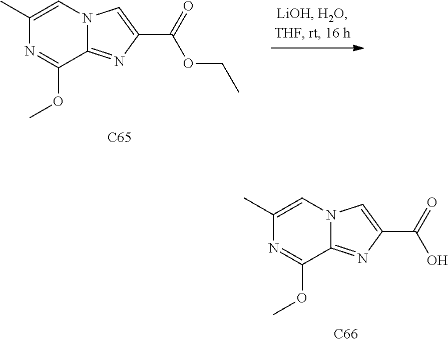 [SMILES: CCOC(=O)c1cn2cc(C)nc(OC)c2n1 ; "C65" ; COc1nc(C)cn2cc(C(=O)O)nc12 ; "C66"]

To a stirred mixture of ethyl 8-methoxy-6-methyl-imidazo[1,2-a]pyrazine-2-carboxylate (1 g, 4.25 mmol) in THF (8 mL) and H₂O (2 mL) was added LiOH (305 mg, 12.75 mmol), and the reaction was stirred for 16 h at room temperature. The resulting mixture was neutralized to pH 5 with 1 N of HCl (aq.). The solid was collected by filtration and washed with water (8 mL). The solid was dried to afford 8-methoxy-6-methyl-imidazo[1,2-a]pyrazine-2-carboxylic acid (0.6 g, 2.90 mmol, 68% yield) as a solid. LCMS (ES, m/z): 208 [M+H]⁺.

### Synthesis of C67

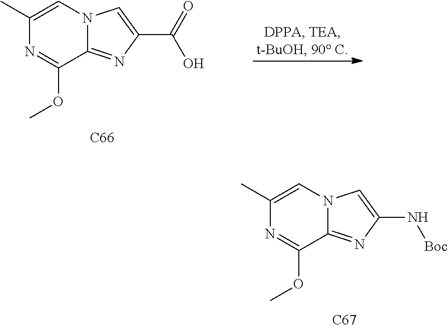 [SMILES: COc1nc(C)cn2cc(C(=O)O)nc12 ; "C66" ; COc1nc(C)cn2cc(NC(=O)OC(C)(C)C)nc12 ; "C67"]

To a solution of 8-methoxy-6-methyl-imidazo[1,2-a]pyrazine-2-carboxylic acid (600 mg, 2.90 mmol) in t-BuOH (10 mL) were added DPPA (956 mg, 3.48 mmol) and TEA (879 mg, 8.69 mmol). The reaction was stirred for 4 h at 90° C. The resulting mixture was concentrated under reduced pressure and purified by silica gel column chromatography (50% EtOAc in PE) to afford tert-butyl N-(8-methoxy-6-methyl-imidazo[1,2-a]pyrazin-2-yl)carbamate (180 mg, 646.8 μmol, 22% yield) as a solid. LCMS (ES, m/z): 279 [M+H]⁺.

### Synthesis of C68

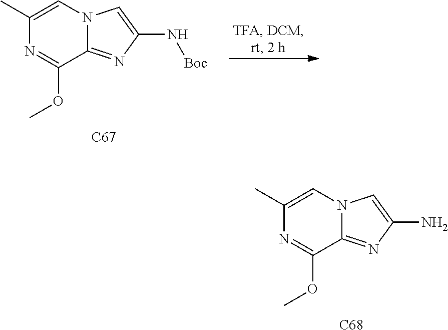 [SMILES: COc1nc(C)cn2cc(NC(=O)OC(C)(C)C)nc12 ; "C67" ; COc1nc(C)cn2cc(N)nc12 ; "C68"]

To a solution of tert-butyl N-(8-methoxy-6-methyl-imidazo[1,2-a]pyrazin-2-yl)carbamate (160 mg, 574.9 μmol) in DCM (3 mL) was added TFA (1 mL). The reaction was stirred for 2 h at rt. The resulting mixture was concentrated under reduced pressure and adjusted to pH 8 with saturated NaHCO₃ (aq.). The resulting mixture was diluted with water (5 mL) and extracted with DCM/MeOH (20/1) (3×10 mL). The combined organic layers were washed with brine (10 mL) and dried over anhydrous Na₂SO₄, and filtered. The filtrate was concentrated under reduced pressure and purified by reversed-phase flash chromatography (Condition 3, Gradient 9) to afford 8-methoxy-6-methyl-imidazo[1,2-a]pyrazin-2-amine (60 mg, 336.7 μmol, 59% yield) as a solid. LCMS (ES, m/z): 179 [M+H]⁺.

An analogous method was followed to obtain the following compounds.

| | Starting | |
|---|---|---|
| Compound | Material | Characterization |
| 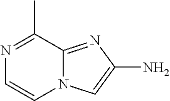 | 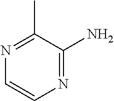 | Modification: Step 1-2) was ran without TFA. Step 2 was ran in MeOH, THE, and H₂O. Step 3 was ran at 85° C. for 16 h. Step 4 was ran using HCl in dioxane and DCM at room temperature for 16 h. The resulting mixture was concentrated under reduced pressure to afford 8-methylimidazo[1,2-a]pyrazin-2-amine |
| | | dihydrochloride (C134, 0.3 g, 1.4 mmol, 84% yield). LCMS |
| C134 | | (ES, m/z): 149 [M + H]⁺. |

### Example 111: Synthesis of C74

Synthesis of C69

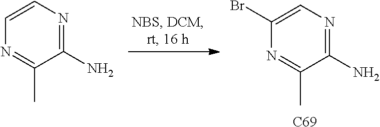

A solution of 3-methylpyrazin-2-amine (35 g, 320.7 mmol) and NBS (57 g, 320.7 mmol) in DCM (400 mL) was stirred for 16 h at room temperature. The reaction was quenched by the addition of water (1 L) at room temperature. The resulting mixture was extracted with DCM (3×400 mL). The combined organic layers were washed with brine (300 mL), dried over anhydrous Na₂SO₄, and filtered. The filtrate was concentrated under reduced pressure and purified by silica gel column chromatography (50% THF in PE) to afford 5-bromo-3-methyl-pyrazin-2-amine (53.6 g, 285.1 mmol, 89% yield) as a solid. LCMS (ES, m/z): 188 [M+H].

### Synthesis of C70

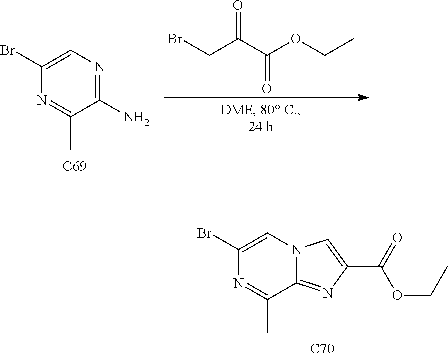

A mixture of 5-bromo-3-methyl-pyrazin-2-amine (59 g, 313.8 mmol) and ethyl 3-bromo-2-oxo-propanoate (61.2 g, 313.8 mmol) in DME (600 mL) was stirred for 24 h at 80° C. under N₂. The mixture was allowed to cool down to room temperature. The reaction was diluted with 50% aqueous NaHCO₃ (2000 mL) at room temperature. The resulting mixture was extracted with EtOAc (3×500 mL). The combined organic layers were washed with brine (2×300 mL), dried over anhydrous Na₂SO₄, and filtered. The filtrate was concentrated under reduced pressure and purified by silica gel column chromatography (50% EtOAc in PE) to afford ethyl 6-bromo-8-methyl-imidazo[1,2-a]pyrazine-2-carboxylate (35.5 g, 125 mmol, 40% yield) as a solid. LCMS (ES, m/z): 284 [M+H].

### Synthesis of C71

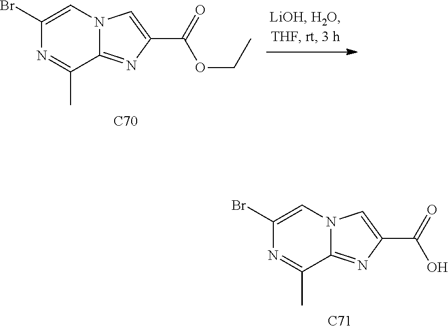 [SMILES: CCOC(=O)c1cn2cc(Br)nc(C)c2n1 ; "C70" ; Cc1nc(Br)cn2cc(C(=O)O)nc12 ; "C71"]

A solution of ethyl 6-bromo-8-methyl-imidazo[1,2-a]pyrazine-2-carboxylate (C70, 2 g, 7.04 mmol) and LiOH (1.69 g, 70.4 mmol) in H₂O (10 mL) and THF (20 mL) was stirred for 3 h at room temperature. The resulting mixture was concentrated under reduced pressure, diluted with water (50 mL), and acidified to pH 6 with citric acid. The resulting mixture was concentrated under reduced pressure and purified by reversed-phase flash chromatography (Condition 8, Gradient 1) to afford 6-bromo-8-methyl-imidazo[1,2-a]pyrazine-2-carboxylic acid (C71, 1.5 g, 5.86 mmol, 83% yield) as a solid. LCMS (ES, m/z): 256 [M+H].

### Synthesis of C72

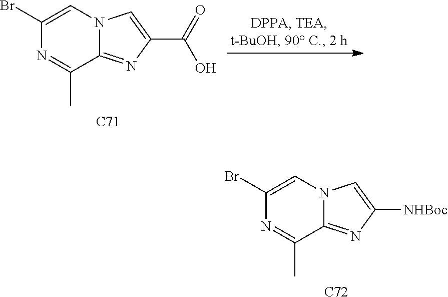 [SMILES: Cc1nc(Br)cn2cc(C(=O)O)nc12 ; "C71" ; Cc1nc(Br)cn2cc(NC(=O)OC(C)(C)C)nc12 ; "C72"]

To a stirred solution of 6-bromo-8-methyl-imidazo[1,2-a]pyrazine-2-carboxylic acid (C71, 1.4 g, 5.47 mmol) and TEA (830 mg, 8.20 mmol) in t-BuOH (15 mL) was added [azido(phenoxy)phosphoryl]oxybenzene (1.8 g, 6.56 mmol, 1.41 mL), and the reaction was stirred for 2 h at 90° C. under N₂. The mixture was allowed to cool to room temperature, diluted with water (100 mL), and extracted with EtOAc (3×30 mL). The combined organic layers were washed with brine (50 mL), dried over anhydrous Na₂SO₄, and filtered. The filtrate was concentrated under reduced pressure and purified by silica gel column chromatography (33% THF in PE) to afford tert-butyl N-(6-bromo-8-methyl-imidazo[1,2-a]pyrazin-2-yl)carbamate (0.92 g, 2.81 mmol, 51% yield) as a solid. LCMS (ES, m/z): 327 [M+H].

### Synthesis of C73

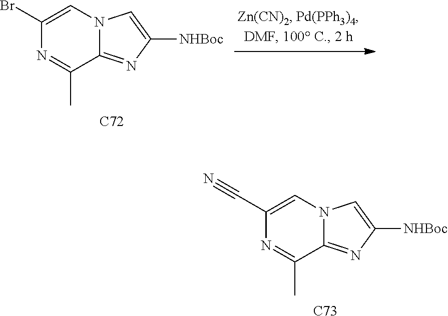 [SMILES: Cc1nc(Br)cn2cc(NC(=O)OC(C)(C)C)nc12 ; "C72" ; Cc1nc(C#N)cn2cc(NC(=O)OC(C)(C)C)nc12 ; "C73"]

To a stirred solution of tert-butyl N-(6-bromo-8-methyl-imidazo[1,2-a]pyrazin-2-yl)-carbamate (C72, 840 mg, 2.6 mmol) and zinc cyanide (302 mg, 2.6 mmol) in DMF (10 mL) was added Pd(PPh₃)₄ (297 mg, 256.7 μmol) at room temperature under N₂. The resulting mixture was stirred for 2 h at 100° C. under N₂. The mixture was allowed to cool down to room temperature. The reaction was diluted with water (50 mL) at room temperature. The resulting mixture was extracted with EtOAc (3×20 mL). The combined organic layers were washed with brine (30 mL), dried over anhydrous Na₂SO₄, and filtered. The filtrate was concentrated under reduced pressure and purified by silica gel column chromatography (33% EtOAc in PE) to afford tert-butyl N-(6-cyano-8-methyl-imidazo[1,2-a]pyrazin-2-yl)carbamate (C73, 610 mg, 2.23 mmol, 87% yield) as a solid. LCMS (ES, m/z): 274 [M+H].

### Synthesis of C74

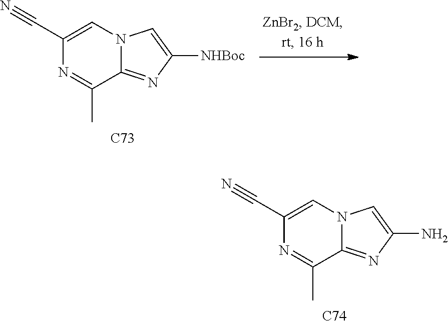 [SMILES: Cc1nc(C#N)cn2cc(NC(=O)OC(C)(C)C)nc12 ; "C73" ; Cc1nc(C#N)cn2cc(N)nc12 ; "C74"]

A solution of tert-butyl N-(6-cyano-8-methyl-imidazo[1,2-a]pyrazin-2-yl)carbamate (C73, 550 mg, 2.01 mmol) and ZnBr₂ (3.17 g, 14.09 mmol) in DCM (10 mL) was stirred for 16 h at room temperature. The resulting mixture was concentrated under reduced pressure and purified by reversed-phase flash chromatography (Condition 3, Gradient 28) to afford 2-amino-8-methyl-imidazo[1,2-a]pyrazine-6-carbonitrile (C74, 140 mg, 808.4 μmol, 40% yield) as a solid. LCMS (ES, m/z): 174 [M+H].

### Example 112: Synthesis of Compound 362

### Synthesis of B100

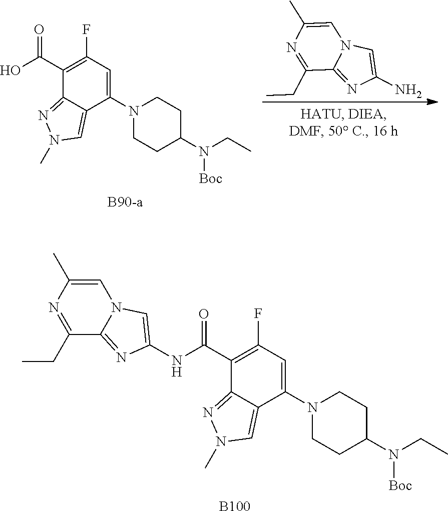 [SMILES: CCN(C(=O)OC(C)(C)C)C1CCN(c2cc(F)c(C(=O)O)c3nn(C)cc23)CC1 ; CCc1nc(C)cn2cc(N)nc12 ; "B100" ; CCc1nc(C)cn2cc(NC(=O)c3c(F)cc(N4CCC(N(CC)C(=O)OC(C)(C)C)CC4)c4cn(C)nc34)nc12]

To a solution of 4-[4-[tert-butoxycarbonyl(ethyl)amino]-1-piperidyl]-6-fluoro-2-methyl-indazole-7-carboxylic acid (100 mg, 237.8 μmol) in DMF (1.5 mL) were added 8-ethyl-6-methyl-imidazo[1,2-a]pyrazin-2-amine (46 mg, 261.6 μmol), DIEA (123 mg, 951.3 μmol, 165.7 μL) and HATU (136 mg, 356.7 μmol), and the mixture was stirred for 16 h at 50° C. The reaction was diluted with water (15 mL) and extracted with EtOAc (2×15 mL). The combined organic layers were washed with water (3×20 mL), dried over anhydrous Na₂SO₄, and filtered. The filtrate was concentrated under reduced pressure and purified by silica gel column chromatography (67% EtOAc in DCM) to afford tert-butyl N-ethyl-N-[1-[7-[(8-ethyl-6-methyl-imidazo[1,2-a]pyrazin-2-yl)carbamoyl]-6-fluoro-2-methyl-indazol-4-yl]-4-piperidyl]carbamate (105 mg, 181.5 μmol, 76% yield) as a solid. LCMS (ES, m/z): 579 [M+H]⁺.

### Synthesis of Compound 362

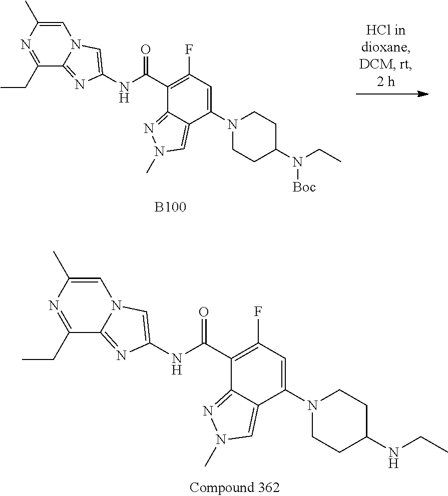

To a solution of tert-butyl N-ethyl-N-[1-[7-[(8-ethyl-6-methyl-imidazo[1,2-a]pyrazin-2-yl)carbamoyl]-6-fluoro-2-methyl-indazol-4-yl]-4-piperidyl]carbamate (100 mg, 172.8 μmol) in DCM (1 mL) was added HCl (4.0 M in 1,4-dioxane, 400 μL), and the reaction was stirred for 2 h at rt. The resulting mixture was concentrated under reduced pressure and purified by Prep-HPLC (Condition 5, Gradient 8) to afford 4-[4-(ethylamino)-1-piperidyl]-N-(8-ethyl-6-methyl-imidazo[1,2-a]pyrazin-2-yl)-6-fluoro-2-methyl-indazole-7-carboxamide (Compound 362, 30 mg, 62.7 μmol, 36% yield) as a solid. LCMS (ES, m/z): 479 [M+H]⁺. ¹H NMR (400 MHz, DMSO-d₆) δ 11.52 (s, 1H), 8.79 (s, 1H), 8.27 (d, J=7.4 Hz, 2H), 6.22 (d, J=16.0 Hz, 1H), 4.22 (s, 3H), 3.90 (dd, J=13.0, 4.1 Hz, 2H), 3.16-3.02 (m, 4H), 2.69 (dd, J=9.3, 5.0 Hz, 1H), 2.58 (q, J=7.1 Hz, 2H), 2.36 (d, J=0.9 Hz, 3H), 1.92 (dd, J=13.2, 3.8 Hz, 2H), 1.41 (dd, J=16.9, 7.4 Hz, 2H), 1.32 (t, J=7.6 Hz, 3H), 1.01 (t, J=7.1 Hz, 3H).

An analogous method was followed to obtain the following compounds.

| Compound | Starting Material | Characterization |
|---|---|---|
| 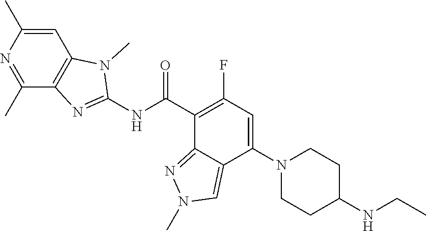 [SMILES: CCNC1CCN(c2cc(F)c(C(=O)Nc3nc4c(C)nc(C)cc4n3C)c3nn(C)cc23)CC1] | 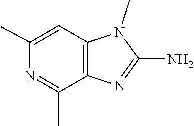 | Modification: Step 1 was ran at 80° C. for 6 h. The crude product was purified by Prep-HPLC (Condition 16, Gradient 1) to afford 4-(4- (ethylamino)-piperidin-1-yl)-6- fluoro-2-methyl-N-(1,4,6- trimethyl-1H-imidazo[4,5- c]pyridin-2-yl)-2H-indazole-7- carboxamide bis(2,2,2- trifluoroacetate) (Compound 402, 35 mg, 49.5 μmol, 30% yield) as a solid. LCMS (ES, m/z): 479 [M + H]⁺ ¹H NMR (400 |
| | | MHz, DMSO-d₆) δ 14.62 (s, 1H), |
| Compound 402 | C144 | 11.80 (s, 1H), 8.91 (s, 1H), 8.48 |
| | | (s, 2H), 7.87 (s, 1H), 6.38 (d, J = |
| | | 15.7 Hz, 1H), 4.20 (s, 3H), 4.11 |
| | | (d, J = 13.4 Hz, 2H), 3.84 (s, 3H). |
| | | 3.14 (t, J = 12.6 Hz, 2H), 3.06- |
| | | 2.98 (m, 2H), 2.85 (s, 3H), 2.70 |
| | | (s, 3H), 2.53-2.50 (m, 1H), 2.12 |
| | | (d, J = 12.2 Hz, 2H), 1.66 (dd, |
| | | J = 13.7, 9.9 Hz, 2H), 1.20 (t, J = |
| | | 7.2 Hz, 3H). |
| 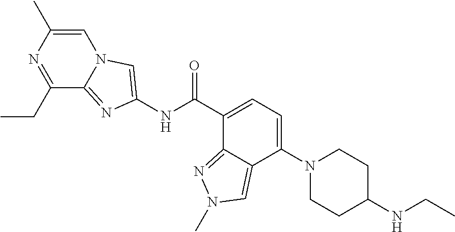 [SMILES: CCNC1CCN(c2ccc(C(=O)Nc3cn4cc(C)nc(CC)c4n3)c3nn(C)cc23)CC1] | 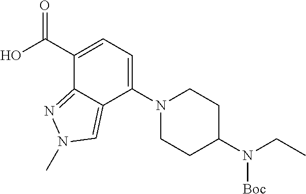 | The crude product was purified by Prep-HPLC (Condition 5, Gradient 17) to afford 4-[4- (ethylamino)-1-piperidy1]-N-(8- ethyl-6-methyl-imidazo[1,2-a]- pyrazin-2-yl)-2-methyl-indazole- 7-carboxamide (Compound 358, 30 mg, 65 μmol, 41% yield) as a solid. LCMS (ES, m/z): 461 [M + H]⁺. ¹H NMR (400 MHz, DMSO-d₆) δ 11.40 (s, 1H), 8.77 (s, 1H), 8.30 (d, J = 4.9 Hz, 2H), 8.00 (d, J = 8.1 Hz, 1H), 6.48 (d, J = 8.2 Hz, 1H), 4.28 (s, 3H), 3.86 |
| | | (dd, J = 10.4, 6.6 Hz, 2H), 3.13- |
| Compound 358 | | 2.99 (m, 4H), 2.65 (t, J = 4.1 Hz, |
| | | 1H), 2.58 (q, J = 7.1 Hz, 2H), |
| | | 2.37 (s, 3H), 1.94 (dd, J = 12.6, |
| | | 3.9 Hz, 2H), 1.58 (s, 1H), 1.41 (d, |
| | | J = 11.2 Hz, 2H), 1.32 (t, J = 7.5 |
| | | Hz, 3H), 1.01 (t, J = 7.1 Hz, 3H). |
| 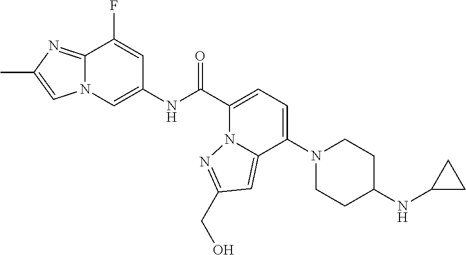 [SMILES: Cc1cn2cc(NC(=O)c3ccc(N4CCC(NC5CC5)CC4)c4cc(CO)nn34)cc(F)c2n1] | 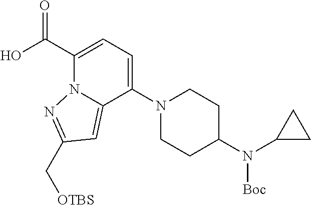 [SMILES: CC(C)(C)OC(=O)N(C1CC1)C1CCN(c2ccc(C(=O)O)n3nc(CO[Si](C)(C)C(C)(C)C)cc23)CC1] | Modification: Step 1 was ran at 40º C for 3 h. Step 2 was ran at room temperature for 1 h. The residue was purified by reversed-phase flash chromatography (Condition 3, Gradient 25) to afford 4-[4- (cyclopropylamino)-1- piperidyl]-N-(8-fluoro-2-methyl- imidazo[1,2-a]-pyridin-6-yl)-2- (hydroxymethyl)pyrazolo-[1,5- alpyridine-7-carboxamide (Compound 622, 0.03 g, 62.8 μmol, 43% yield) as a solid. LCMS (ES, m/z): 478 [M + H]⁻. ¹H NMR (400 MHz, DMSO-d₆) |
| | | δ 12.66 (s, 1H), 9.22 (d, J = 1.7 |
| Compound 622 | B153 | Hz, 1H), 7.93 (d, J = 3.1 Hz, 1H), |
| | | 7.79 (d, J = 8.1 Hz, 1H), 7.40 (dd, |
| | 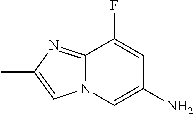 | J = 12.3, 1.7 Hz, 1H), 6.80-6.73 (m, 2H), 5.48 (s, 1H), 4.82 (s, 2H), 3.77-3.70 (m, 2H), 3.02- 2.92 (m, 2H), 2.78 (p, J = 5.6 Hz, 1H), 2.36 (s, 3H), 2.14 (tt, J = 6.7, 3.6 Hz, 1H), 2.03 (d, J = 12.3 Hz, 2H), 1.54-1.45 (m, 2H), 0.40 (dt, |
| | | J = 6.2, 3.0 Hz, 2H), 0.29-0.21 |
| | | (m, 2H). |
| 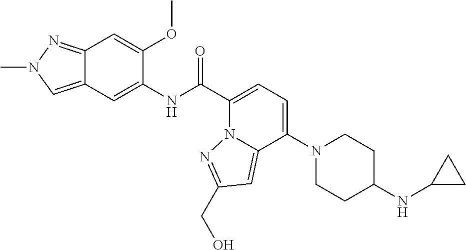 | 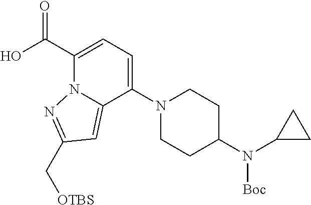 [SMILES: CC(C)(C)OC(=O)N(C1CC1)C1CCN(c2ccc(C(=O)O)n3nc(CO[Si](C)(C)C(C)(C)C)cc23)CC1] | Modification: Step 1 was ran for 3 h at room temperature. Step 2 was ran for 1 h at room temperature. The residue was purified by Prep- HPLC (Condition 2, Gradient 5) to afford4-(4- (cyclopropylamino)piperidin-1- yl)-2-(hydroxymethyl)-N-(6- methoxy-2-methyl-2H-indazol- 5-yl)pyrazolo[1,5-a]pyridine-7- carboxamide 2,2,2- trifluoroacetate (Compound 443, 0.02 g, 33.1 μmol, 29% yield) as a solid. LCMS (ES, |
| | | m/z): 490 [M + H]⁺. ¹H NMR (300 |
| Compound 443 | B153 | MHz, DMSO-d₆) δ 13.19 (s, 1H), |
| | | 8.95-8.81 (m, 2H), 8.85 (s, 1H), |
| | 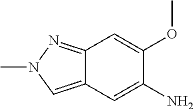 | 8.24 (s, 1H), 7.87 (d, J = 8.0 Hz, 1H), 7.11 (s, 1H), 6.88-6.79 (m, 2H), 4.85 (s, 2H), 4.09 (s, 3H), 4.05 (s, 3H), 3.90 (d, J = 12.7 Hz, 3H), 3.52-3.45 (m, 1H), 2.96 (t, J = 12.2 Hz, 2H), 2.80-2.72 (m, 1H), 2.24 (d, J = 11.5 Hz, 2H), |
| | | 1.81 (q, J = 12.2, 11.6 Hz, 2H), |
| | | 0.85 (d, J =5.5 Hz, 4H). |
| 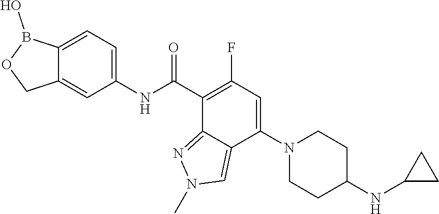 [SMILES: Cn1cc2c(N3CCC(NC4CC4)CC3)cc(F)c(C(=O)Nc3ccc4c(c3)COB4O)c2n1] | 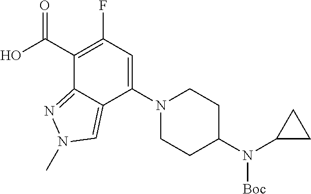 [SMILES: Cn1cc2c(N3CCC(N(C(=O)OC(C)(C)C)C4CC4)CC3)cc(F)c(C(=O)O)c2n1] | Modification: Step 1 was ran at room temperature. Step 2 was ran in EtOAc. The residue was purified by reversed-phase flash chromatography (Condition 9, Gradient 1) to afford 4-(4- (cyclopropyl-amino)piperidin-1- yl)-6-fluoro-N-(1-hydroxy-1,3- dihydrobenzo[c][1,2]-oxaborol- 5-y1)-2-methyl-2H-indazole-7- carboxamide 2,2,2- trifluoroacetate (Compound |
| | | 481, 26 mg, 45.0 μmol). LCMS |
| Compound 481 | 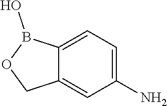 [SMILES: Nc1ccc2c(c1)COB2O] | (ES, m/z): 464.3 [M + H − TFA]⁻. ¹H NMR (400 MHz, DMSO-d₆) δ 11.10 (s, 1H), 9.01-8.80 (m, 3H), 7.93 (s, 1H), 7.70 (d, J = 8.0 Hz, 1H), 7.63-7.58 (m, 1H), 6.33 (d, J = 15.2 Hz, 1H), 5.00 (s, 2H), 4.21 (s, 3H), 4.04-4.00 (m, 2H), |
| | | 3.49 (brs, 1H), 3.06 (t, J = 12.4 |
| | | Hz, 2H), 2.80-2.70 (m, 1H), 2.19- |
| | | 2.17 (m, 2H), 1.76-1.68 (m, 2H), |
| | | 0.90-0.80 (m, 4H). |
| 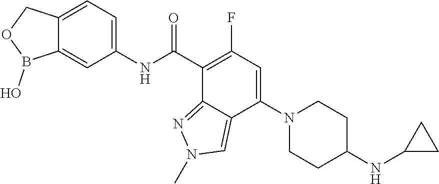 [SMILES: Cn1cc2c(N3CCC(NC4CC4)CC3)cc(F)c(C(=O)Nc3ccc4c(c3)B(O)OC4)c2n1] | 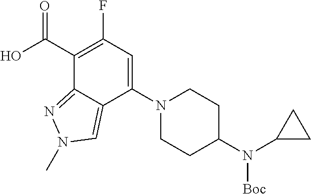 [SMILES: Cn1cc2c(N3CCC(N(C(=O)OC(C)(C)C)C4CC4)CC3)cc(F)c(C(=O)O)c2n1] | Modification: Step 1 was ran at room temperature. Step 2 was ran in EtOAc. The residue was purified by reversed-phase flash chromatography (Condition 9, Gradient 1) to afford 4-[4- (cyclopropyl-amino)-1- piperidy1]-6-fluoro-N-(1- hydroxy-3H-2, 1-benzoxaborol- 6-y1)-2-methyl-indazole-7- carboxamide 2,2,2- trifluoroacetate (Compound |
| | | 482, 28 mg, 48.5 μmol). LCMS |
| Compound 482 | 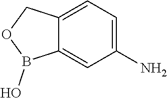 [SMILES: Nc1ccc2c(c1)B(O)OC2] | (ES, m/z): 464.3 [M + H − TFA]⁻. ¹H NMR (400 MHz, DMSO-d₆) δ 10.94 (s, 1H), 9.24 (brs, 1H), 8.84 (brs, H), 8.80 (s, 1H), 8.15 (s, 1H), 7.86-7.75 (m, 1H), 7.40 (d, J = 8.4 Hz, 1H), 6.32 (d, J = |
| | | 14.8 Hz, 1H), 4.98 (s, 2H), 4.21 |
| | | (s, 3H), 4.03 (d, J = 12.8 Hz, 2H), |
| | | 3.50-3.40 (m, 1H), 3.07-3.01 (m, |
| | | 2H), 2.85-2.75 (m, 1H), 2.20- |
| | | 2.10 (m, 2H), 1.83-1.63 (m, 2H), |
| | | 0.90-0.80 (m, 4H). |
| 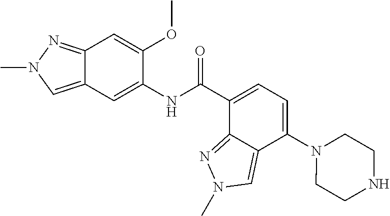 [SMILES: COc1cc2nn(C)cc2cc1NC(=O)c1ccc(N2CCNCC2)c2cn(C)nc12] | 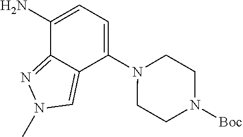 | Modification: Sep 1 was ran for 2 h at room temperature. The residue was purified by reversed-phase flash chromatography (Condition 3, Gradient 11) to afford 6- methoxy-2-methyl-N-(2-methyl- 4-piperazin-1-yl-indazol-7-yl)- indazole-5-carboxamide (Compound 489, 22.3 mg, 53.2 μmol). LCMS (ES, m/z): 420 [M + H]⁺. ¹H NMR (300 MHz, DMSO-d₆) δ 11.03 (s, 1H), 8.61 |
| | | (s, 1H), 8.50 (s, 1H), 8.46 (s, 1H), |
| Compound 489 | 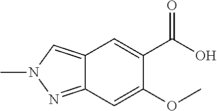 | 8.07 (d, J = 8.1 Hz, 1H), 7.22 (s, 1H), 6.30 (d, J = 7.8 Hz, 1H), 4.21 (s, 3H), 4.18 (s, 3H), 4.16 (s, 3H), 3.07-3.05 (m, 4H), 2.95- 2.85 (m, 4H). |
| 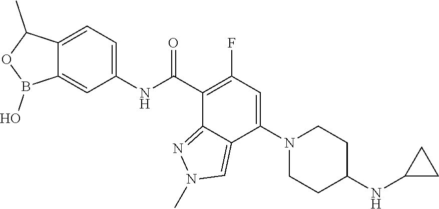 [SMILES: CC1OB(O)c2cc(NC(=O)c3c(F)cc(N4CCC(NC5CC5)CC4)c4cn(C)nc34)ccc21] | 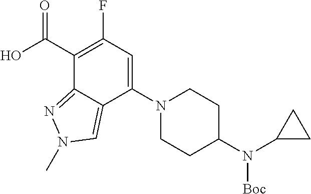 [SMILES: Cn1cc2c(N3CCC(N(C(=O)OC(C)(C)C)C4CC4)CC3)cc(F)c(C(=O)O)c2n1] | Modification: Step 1 was ran at room temperature. Step 2 was ran in EtOAc. The residue was purified by reversed-phase flash chromatography (Condition 9, Gradient 1) to afford 4-(4- (cyclopropylamino)piperidin-1- yl)-6-fluoro-N-(1-hydroxy-3- methyl-1,3-dihydrobenzo[c]- [1,2]oxaborol-6-yl)-2-methyl- 2H-indazole-7-carboxamide 2,2,2-trifluoroacetate |
| | | (Compound 490, 27.1 mg, 45.8 |
| Compound 490 | 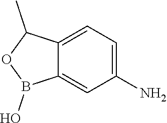 | μmol). LCMS (ES, m/z): 478.2 [M + H − TFA]⁺. ¹H NMR (400 MHz, DMSO-d₆) δ 10.93 (s, 1H), 8.93 (brs, 2H), 8.80 (s, 1H), 8.09 (s, 1H), 7.83-7.75 (m, 1H), 7.38 (d, J = 8.4 Hz, 1H), 6.32 (d, J = 14.8 Hz, 1H), 5.26-5.15 (m, 1H), 4.20 (s, 3H), 4.03 (d, J = 12.8 Hz, |
| | | 2H), 3.48 (brs, 1H), 3.04 (t, J = |
| | D33 | 12.4 Hz, 2H), 2.82-2.75 (m, 1H), |
| | | 2.20-2.18 (m, 2H), 1.82-1.65 (m, |
| | | 2H), 1.41 (d, J = 6.4 Hz, 3H), |
| | | 0.90-0.78 (m, 4H) |
| 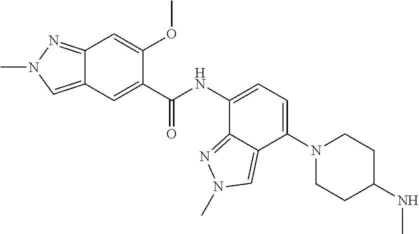 | 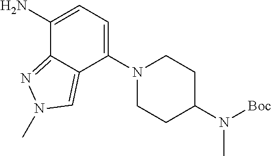 | Modification: Step 1 was ran for 1 h at room temperature. Step 2 was ran in EtOAc. The crude product was purified by Prep-HPLC (Condition 18, Gradient 2) to afford 6-methoxy- 2-methyl-N-[2-methyl-4-[4- (methylamino)-1- piperidyl]indazol-7-yl]-indazole- 5-carboxamide (Compound 521, 28.6 mg, 63.9 μmol). LCMS (ES, m/z): 448 [M + H]⁺. ¹H NMR (400 MHz, DMSO-d₆) δ 11.01 (s, 1H), 8.60 (s, 1H), 8.49 (s, 1H), 8.42 (s, |
| | | 1H), 8.05 (d, J = 8.0 Hz, 1H), |
| Compound 521 | 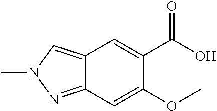 | 7.21 (s, 1H), 6.30 (d, J = 8.0 Hz, 1H), 4.21 (s, 3H), 4.17 (s, 6H), 3.59-3,56 (m, 2H), 2.76-2.71 (m, 2H), 2.46-2.42 (m, 1H), 2.31 (s, 3H), 1.96-1.93 (m, 2H), 1.49- 1.42 (m, 2H). |
| 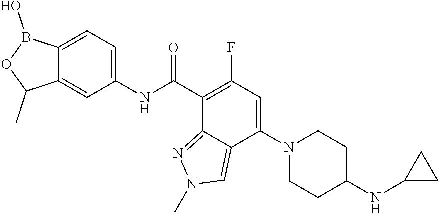 [SMILES: CC1OB(O)c2ccc(NC(=O)c3c(F)cc(N4CCC(NC5CC5)CC4)c4cn(C)nc34)cc21] | 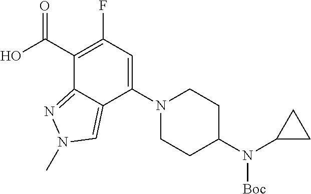 [SMILES: Cn1cc2c(N3CCC(N(C(=O)OC(C)(C)C)C4CC4)CC3)cc(F)c(C(=O)O)c2n1] | Modification: Step 1 was ran at room temperature. Step 2 was ran in EtOAc for 16 h. The reaction mixture was stirred for 16 h at room temperature, then concentrated, diluted with EtOAc (10 mL), and stirred for another 1 h at room temperature. The solid was collected by filtration to afford 4-(4- (cyclopropyl-amino)piperidin-1- yl)-6-fluoro-N-(1-hydroxy-3- methyl-1,3-dihydrobenzo[c]- |
| | | [1,2]oxaborol-5-y1)-2-methyl- |
| Compound 558 | 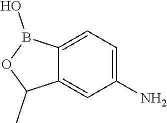 | 2H-indazole-7-carboxamide hydrochloride (Compound 558, 36 mg, 70.1 μmol). LCMS (ES, m/z): 478 [M + H]⁺. ¹H NMR (400 MHz, DMSO-d₆) δ 11.09 (s, 1H), 9.29 (brs, 2H), 8.81 (s, 1H), 7.93 (s, 1H), 7.66 (d, J = 8.0 Hz, 1H), 7.55 (d, J = 8.0 Hz, 1H), 6.31 (d, |
| | | J = 15.2 Hz, 1H), 5.24-5.19 (m, |
| | D38 | 1H), 4.21 (s, 3H), 4.05-3.92 (m, |
| | | 2H), 3.44 (brs, 1H), 3.07-3.00 (m, |
| | | 2H), 2.74 (brs, 1H), 2.33-2.21 (m, |
| | | 2H), 1.82-1.76 (m, 2H), 1.41 (d, |
| | | J = 8.0 Hz, 3H), 1.41-1.39 (m, 2H), |
| | | 1.24-1.19 (m, 2H). |
| 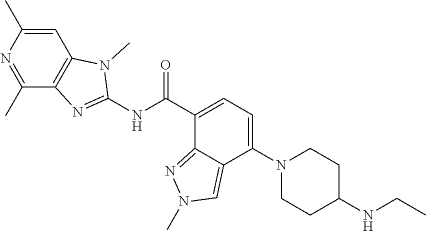 [SMILES: CCNC1CCN(c2ccc(C(=O)Nc3nc4c(C)nc(C)cc4n3C)c3nn(C)cc23)CC1] | 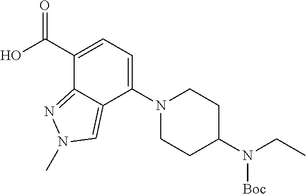 | Modification: Step 1 was ran at 80° C. for 3 days. Step 2 was ran at room temperature for 2 h. The crude product was purified by Prep-HPLC (Condition 5, Gradient 12a) to afford 4-[4- (ethylamino)-1-piperidyl]-2- methyl-N-(1,4,6-trimethyl- imidazo[4,5-c]pyridin-2-y1)- indazole-7-carboxamide (Compound 583, 50 mg, 108.6 μmol). LCMS (ES, m/z): 461 [M + H]⁺. ¹H NMR (400 MHz, DMSO-d₆) δ 11.21 (s, 1H), 8.80 |
| | | (s, 1H), 7.98 (d, J = 8.2 Hz, 1H), |
| Compound 583 | 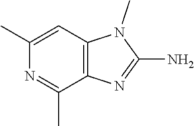 | 7.25 (s, 1H), 6.49 (d, J = 8.3 Hz, 1H), 4.23 (s, 3H), 3.91 (dd, J = 10.6, 6.7 Hz, 2H), 3.65 (s, 3H), 3.15-3.04 (m, 2H), 2.74-2.63 (m, 1H), 2.60 (d, J = 7.9 Hz, 5H), 2.51 (s, 3H), 1.95 (dd, J = 13.2, 3.9 Hz, 2H), 1.53-1.31 (m, 2H), 1.02 (t, J = 7.1 Hz, 3H). |

### Example 113: Synthesis of C79

### Synthesis of C75

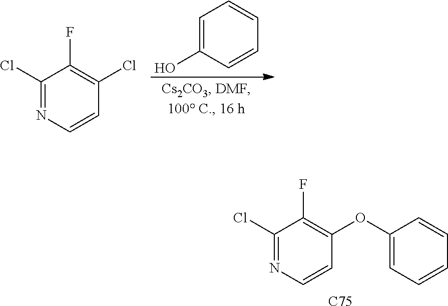

To a stirred solution of 2,4-dichloro-3-fluoro-pyridine (4 g, 24.1 mmol) and phenol (2.27 g, 24.1 mmol, 2.12 mL) in DMF (50 mL) was added Cs₂CO₃ (15.7 g, 48.2 mmol), and the reaction was stirred for 16 h at 100° C. The reaction was diluted with water (300 mL) at room temperature and extracted with EtOAc (3×100 mL). The combined organic layers were washed with brine (50 mL), dried over anhydrous Na₂SO₄, and filtered. The filtrate was concentrated under reduced pressure and purified by silica gel column chromatography (5% THF in PE) to afford 2-chloro-3-fluoro-4-phenoxy-pyridine (1.2 g, 5.37 mmol, 22% yield) as an oil. LCMS (ES, m/z): 233 [M+H].

### Synthesis of C76

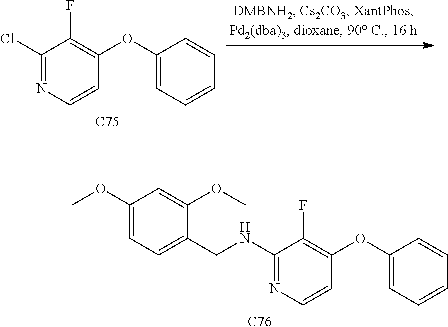 [SMILES: Fc1c(Oc2ccccc2)ccnc1Cl ; "C75" ; COc1ccc(CNc2nccc(Oc3ccccc3)c2F)c(OC)c1 ; "C76"]

To a stirred solution of 2-chloro-3-fluoro-4-phenoxy-pyridine (1.6 g, 7.15 mmol), Cs₂CO₃ (4.66 g, 14.31 mmol) and (2,4-dimethoxyphenyl) methanamine (1.2 g, 7.15 mmol) in dioxane (20 mL) were added XantPhos (828 mg, 1.43 mmol) and Pd₂(dba)₃ (655 mg, 715.5 μmol) at room temperature under N₂. The resulting mixture was stirred for 16 h at 90° C. under N₂. The mixture was concentrated under reduced pressure and purified by silica gel column chromatography (5% THF in PE) to afford N-[(2,4-dimethoxyphenyl)methyl]-3-fluoro-4-phenoxy-pyridin-2-amine (1.5 g, 4.23 mmol, 59% yield) as an oil. LCMS (ES, m/z): 355 [M+H].

### Synthesis of C77

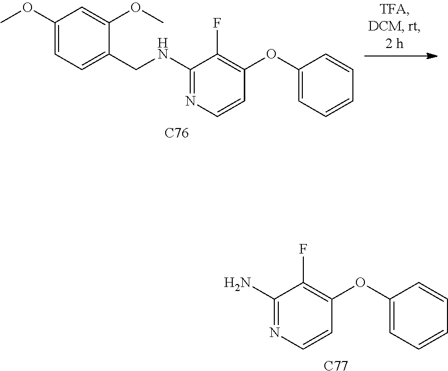 [SMILES: COc1ccc(CNc2nccc(Oc3ccccc3)c2F)c(OC)c1 ; "C76" ; Nc1nccc(Oc2ccccc2)c1F ; "C77"]

A solution of N-[(2,4-dimethoxyphenyl)methyl]-3-fluoro-4-phenoxy-pyridin-2-amine (1.46 g, 4.12 mmol) and TFA (5 mL) in DCM (15 mL) was stirred for 2 h at room temperature. The reaction was quenched by the addition of 20% NaHCO₃ (100 mL) at room temperature. The resulting mixture was extracted with EtOAc (3×30 mL). The combined organic layers were washed with brine (20 mL), dried over anhydrous Na₂SO₄, and filtered. The filtrate was concentrated under reduced pressure and purified by silica gel column chromatography (33% THF in PE) to afford 3-fluoro-4-phenoxy-pyridin-2-amine (735 mg, 3.60 mmol, 87% yield) as a solid. LCMS (ES, m/z): 205 [M+H].

### Synthesis of C78

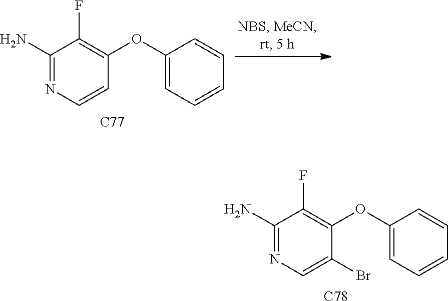 [SMILES: Nc1nccc(Oc2ccccc2)c1F ; "C77" ; Nc1ncc(Br)c(Oc2ccccc2)c1F ; "C78"]

A solution of 3-fluoro-4-phenoxy-pyridin-2-amine (700 mg, 3.43 mmol) and 1-bromopyrrolidine-2,5-dione (732 mg, 4.11 mmol) in MeCN (10 mL) was stirred for 5 h at room temperature. The reaction was quenched by the addition of water (100 mL) at room temperature. The resulting mixture was extracted with EtOAc (3×30 mL). The combined organic layers were washed with brine (30 mL), dried over anhydrous Na₂SO₄, and filtered. The filtrate was concentrated under reduced pressure and purified by silica gel column chromatography (25% THF in PE) to afford 5-bromo-3-fluoro-4-phenoxy-pyridin-2-amine (650 mg, 2.30 mmol, 67% yield) as a solid. LCMS (ES, m/z): 283 [M+H].

### Synthesis of C79

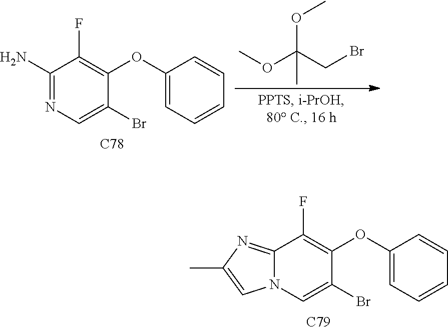

To a stirred solution of 5-bromo-3-fluoro-4-phenoxy-pyridin-2-amine (650 mg, 2.30 mmol) and 1-bromo-2,2-dimethoxy-propane (630 mg, 3.44 mmol) in i-PrOH (10 mL) was added PPTs (58 mg, 229.6 μmol) at room temperature under N₂. The resulting mixture was stirred for 16 h at 80° C. under N₂. The mixture was allowed to cool down to room temperature. The reaction was quenched by the addition of 20% NaHCO₃ (100 mL) at room temperature. The resulting mixture was extracted with EtOAc (3×40 mL). The combined organic layers were washed with brine (20 mL), dried over anhydrous Na₂SO₄, and filtered. The filtrate was concentrated under reduced pressure and purified by silica gel column chromatography (17% THF in PE) to afford 6-bromo-8-fluoro-2-methyl-7-phenoxy-imidazo[1,2-a]pyridine (420 mg, 1.31 mmol, 57% yield) as a solid. LCMS (ES, m/z): 321 [M+H].

An analogous method was followed to obtain the following compound.

| | Starting | |
|---|---|---|
| Compound | Material | Characterization |
| 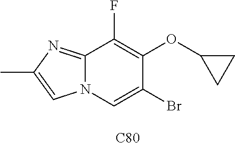 [SMILES: Cc1cn2cc(Br)c(OC3CC3)c(F)c2n1 ; "C80"] | 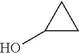 | The residue was purified by silica gel column chromatography (17% THF in PE) to afford 6-bromo-7- (cyclopropoxy)-8-fluoro-2-methyl-imidazo[1,2-a]-pyridine (370 mg, 1.3 mmol, 80% yield) as a solid. LCMS (ES, m/z): 285 [M + H]. |

### Example 114: Synthesis of C88

### Synthesis of C85

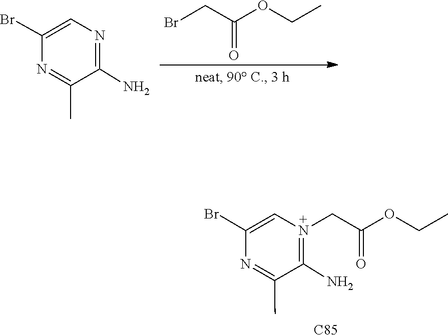

To a stirred solution of 5-bromo-3-methyl-pyrazin-2-amine (2 g, 10.64 mmol, 1 eq) in ethyl 2-bromoacetate (10.66 g, 63.82 mmol, 6 eq), and the reaction was stirred for 3 h at 90° C. The reaction mixture was cooled to room temperature. The precipitated solids were collected by filtration, washed with EtOAc (3×100 mL), and dried to afford ethyl 2-(2-amino-5-bromo-3-methyl-pyrazin-1-ium-1-yl)acetate (3.3 g, crude) as a solid. LCMS (ES, m/z): 275 [M+H]⁺.

### Synthesis of C86

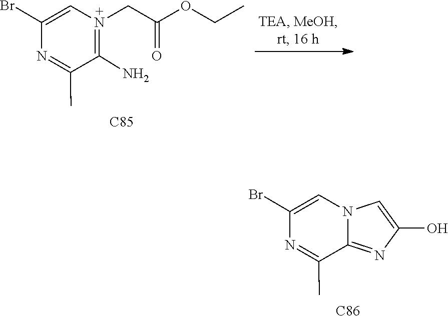

To a stirred solution of ethyl 2-(2-amino-5-bromo-3-methyl-pyrazin-1-ium-1-yl)acetate (3.2 g, 11.63 mmol, 1 eq) in MeOH (60 mL) was added TEA (3.53 g, 34.89 mmol, 4.86 mL, 3 eq), and the reaction was stirred for 4 h at room temperature. The precipitated solids were collected by filtration, washed with EtOAc (3×100 mL), and dried to afford 6-bromo-8-methyl-imidazo[1,2-a]pyrazin-2-ol (C86, 1.5 g, 57% yield) as a solid. LCMS (ES, m/z): 228 [M+H]⁺.

### Synthesis of C87

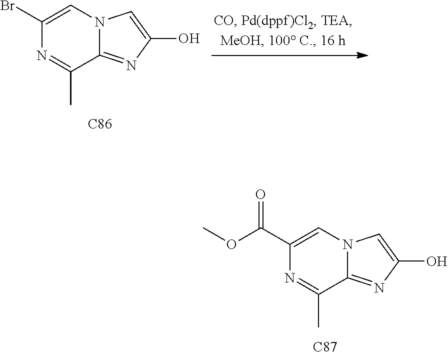

To a solution of 6-bromo-8-methyl-imidazo[1,2-a]pyrazin-2-ol (1.3 g, 5.70 mmol, 1 eq) in MeOH (30 mL) were added TEA (1.73 g, 17.1 mmol, 3 eq) and Pd(dppf)Cl₂ (417 mg, 570.1 μmol, 0.1 eq) in a pressure tank under N₂. The mixture was pressurized to 30 atm with carbon monoxide at 100° C. for 16 h. The reaction mixture was cooled to room temperature and concentrated under reduced pressure and purified by silica gel column chromatography (50% THF in PE) to afford methyl 2-hydroxy-8-methyl-imidazo[1,2-a]pyrazine-6-carboxylate (C87, 1.1 g, 93% yield) as solid. LCMS (ES, m/z): 208 [M+H]⁺.

### Synthesis of C88

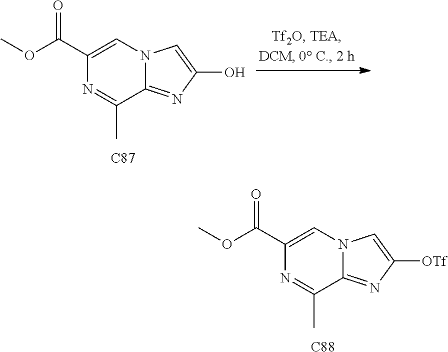 [SMILES: COC(=O)c1cn2cc(O)nc2c(C)n1 ; "C87" ; COC(=O)c1cn2cc(OS(=O)(=O)C(F)(F)F)nc2c(C)n1 ; "C88"]

To a stirred solution of methyl 2-hydroxy-8-methyl-imidazo[1,2-a]pyrazine-6-carboxylate (600 mg, 2.90 mmol, 1 eq) in DCM (12 mL) was added TEA (586 mg, 5.79 mmol, 2 eq) and Tf₂O (1.23 g, 4.34 mmol, 1.5 eq), and the reaction was stirred for 2 h at 0° C. The resulting mixture was diluted with H₂O (20 mL) and extracted with DCM (3×50 mL). The combined organic layers were washed with brine (50 mL), dried over anhydrous Na₂SO₄, and filtered. The filtrate was concentrated under reduced pressure and purified by silica gel column chromatography (50% THF in PE) to afford methyl 8-methyl-2-(trifluoromethylsulfonyloxy)imidazo[1,2-a]pyrazine-6-carboxylate (C88, 278 mg, 28% yield) as a solid. LCMS (ES, m/z): 340 [M+H]⁺.

### Example 115: Synthesis of Compound 337

### Synthesis of B103

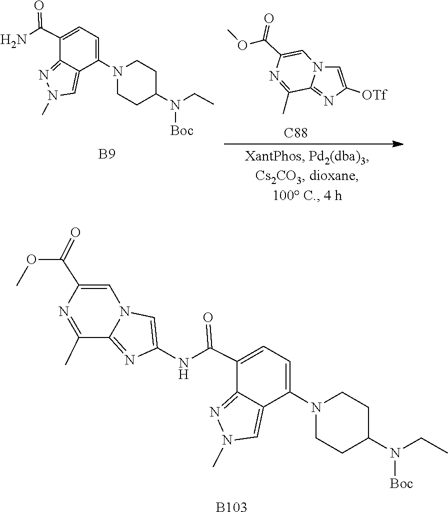

To a stirred solution of tert-butyl N-[1-(7-carbamoyl-2-methyl-indazol-4-yl)-4-piperidyl]-N-ethyl-carbamate (200 mg, 498.1 μmol, 1 eq) and methyl 8-methyl-2-(trifluoromethylsulfonyloxy)imidazo[1,2-a]pyrazine-6-carboxylate (253 mg, 747.2 μmol, 1.5 eq) in dioxane (4 mL) were added Cs₂CO₃ (486 mg, 1.49 mmol, 3 eq), Xantphos (57 mg, 99.63 μmol, 0.2 eq) and Pd₂(dba)₃ (45 mg, 49.8 μmol, 0.1 eq) at room temperature under N₂. The resulting mixture was stirred for 4 h at 100° C. under N₂. The resulting mixture was concentrated under reduced pressure and purified by silica gel column chromatography (9% MeOH in DCM) to afford methyl 2-[[4-[4-[tert-butoxycarbonyl(ethyl)amino]-1-piperidyl]-2-methyl-indazole-7-carbonyl]amino]-8-methyl-imidazo[1,2-a]pyrazine-6-carboxylate (278 mg, 78% yield) as a solid. LCMS (ES, m/z): 591 [M+H]⁺.

### Synthesis of B104

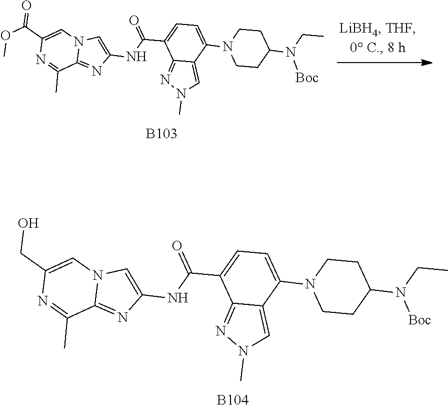 [SMILES: "B103" ; "B104" ; CCN(C(=O)OC(C)(C)C)C1CCN(c2ccc(C(=O)Nc3cn4cc(C(=O)OC)nc(C)c4n3)c3nn(C)cc23)CC1 ; CCN(C(=O)OC(C)(C)C)C1CCN(c2ccc(C(=O)Nc3cn4cc(CO)nc(C)c4n3)c3nn(C)cc23)CC1]

To a stirred solution of methyl 2-[[4-[4-[tert-butoxycarbonyl(ethyl)amino]-1-piperidyl]-2-methyl-indazole-7-carbonyl]amino]-8-methyl-imidazo[1,2-a]pyrazine-6-carboxylate (278 mg, 470.7 μmol, 1 eq) in THF (5 mL) was added LiBH₄ (2.0 M solution in THF) (1.2 ml, 2.35 mmol, 5 eq) at 0° C., and the reaction was stirred for 8 h at 0° C. The resulting mixture was diluted with H₂O (20 mL) and extracted with DCM (3×50 mL). The combined organic layers were washed with brine (50 mL), dried over anhydrous Na₂SO₄, and filtered. The filtrate was concentrated under reduced pressure and purified by silica gel column chromatography (9% MeOH in DCM) to afford tert-butyl N-ethyl-N-[1-[7-[[6-(hydroxymethyl)-8-methyl-imidazo[1,2-a]pyrazin-2-yl]carbamoyl]-2-methyl-indazol-4-yl]-4-piperidyl]carbamate (87 mg, 33% yield) as a solid. LCMS (ES, m/z): 563 [M+H]⁺.

### Synthesis of Compound 337

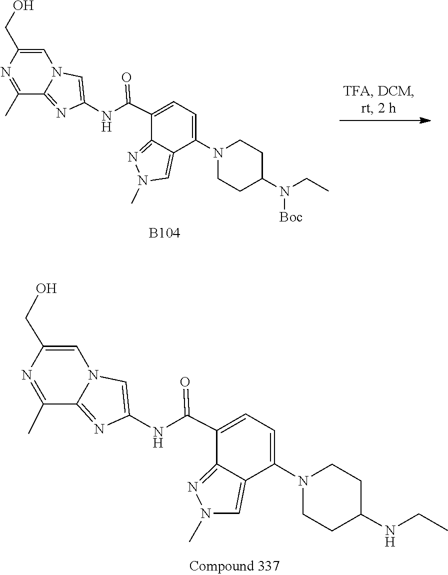

To a stirred solution of tert-butyl N-ethyl-N-[1-[7-[[6-(hydroxymethyl)-8-methyl-imidazo[1,2-a]pyrazin-2-yl]carbamoyl]-2-methyl-indazol-4-yl]-4-piperidyl]carbamate (35 mg, 62.2 μmol, 1 eq) in DCM (1 mL) was added TFA (0.5 mL), and the reaction was stirred for 2 h at room temperature. The resulting mixture was concentrated under reduced pressure and purified by Prep-HPLC (Condition 15, Gradient 1) to afford 4-(4-(ethylamino)piperidin-1-yl)-N-(6-(hydroxymethyl)-8-methylimidazo[1,2-a]pyrazin-2-yl)-2-methyl-2H-indazole-7-carboxamide 2,2,2-trifluoroacetate (Compound 337, 24.4 mg, 65% yield) as solid. LCMS (ES, m/z): 463 [M+H]⁺. ¹H NMR (400 MHz, DMSO-d₆) δ 11.48 (s, 1H), 8.87 (s, 1H), 8.53 (s, 2H), 8.46 (d, J=8.0 Hz, 2H), 8.06 (d, J=8.0 Hz, 1H), 6.58 (d, J=8.2 Hz, 1H), 4.56 (d, J=1.2 Hz, 2H), 4.32 (s, 3H), 4.06 (d, J=13.0 Hz, 2H), 3.36 (s, 1H), 3.06 (t, J=11.6 Hz, 4H), 2.73 (s, 3H), 2.19-2.11 (m, 2H), 1.78-1.65 (m, 2H), 1.23 (t, J=7.2 Hz, 4H).

### Example 116: Synthesis of C90

### Synthesis of C89

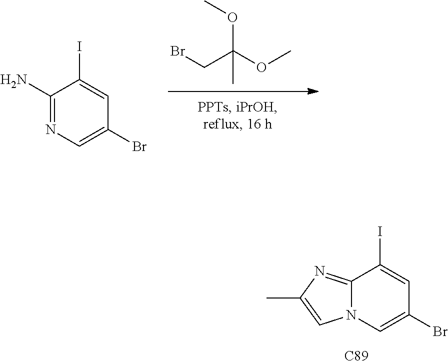

To a stirred solution of 5-bromo-3-iodo-pyridin-2-amine (4 g, 13.38 mmol) and 1-bromo-2,2-dimethoxy-propane (2.94 g, 16.06 mmol) in i-PrOH (80 mL) was added PPTS (336 mg, 1.34 mmol), and the reaction was stirred for 16 h at 90° C. under N₂ atmosphere. The resulting mixture was diluted with water (60 mL) and extracted with EtOAc (3×50 mL). The combined organic layers were washed with water (2×70 mL) and brine (70 mL), dried over anhydrous Na₂SO₄, and filtered. The filtrate was concentrated under reduced pressure and purified by silica gel column chromatography (33% EtOAc in PE) to afford 6-bromo-8-iodo-2-methyl-imidazo[1,2-a]pyridine (4 g, 11.87 mmol, 89% yield) as a solid. LCMS (ES, m/z): 337 [M+H]⁺.

### Synthesis of C90

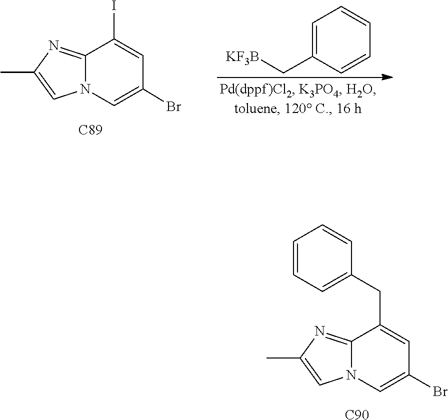

To a stirred mixture of benzyl(trifluoro) boranuide potassium (470 mg, 2.37 mmol) and 6-bromo-8-iodo-2-methyl-imidazo[1,2-a]pyridine (400 mg, 1.19 mmol) in toluene (8 mL) and H₂O (1 mL) were added K₃PO₄ (503 mg, 2.37 mmol) and Pd(dppf)Cl₂ (86 mg, 118.7 μmol), and the reaction was stirred for 16 h at 120° C. under N₂. The resulting mixture was cooled to room temperature, diluted with water (30 mL), and extracted with EtOAc (2×50 mL). The combined organic layers were washed with brine (60 mL), dried over anhydrous Na₂SO₄, and filtered. The filtrate was concentrated under reduced pressure and purified by silica gel column chromatography (50% EtOAc in PE) to afford 8-benzyl-6-bromo-2-methyl-imidazo[1,2-a]pyridine (150 mg, 498 μmol, 42% yield) as a solid. LCMS (ES, m/z): 301 [M+H]⁺.

### Example 117: Synthesis of Compound 350

### Synthesis of B105

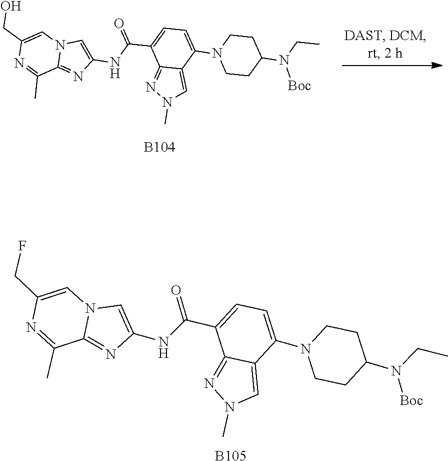 [SMILES: "B104" ; "B105" ; CCN(C(=O)OC(C)(C)C)C1CCN(c2ccc(C(=O)Nc3cn4cc(CO)nc(C)c4n3)c3nn(C)cc23)CC1 ; CCN(C(=O)OC(C)(C)C)C1CCN(c2ccc(C(=O)Nc3cn4cc(CF)nc(C)c4n3)c3nn(C)cc23)CC1]

To a stirred solution of tert-butyl N-ethyl-N-[1-[7-[[6-(hydroxymethyl)-8-methyl-imidazo[1,2-a]pyrazin-2-yl]carbamoyl]-2-methyl-indazol-4-yl]-4-piperidyl]carbamate (50 mg, 88.86 μmol, 1 eq) in DCM (2 mL) was added DAST (42 mg, 266.6 μmol, 3 eq) at 0° C., and the reaction was stirred for 2 h at room temperature. The resulting mixture was diluted with H₂O (10 mL) and extracted with DCM (3×10 mL). The combined organic layers were washed with brine (10 mL), dried over anhydrous Na₂SO₄, and filtered. The filtrate was concentrated under reduced pressure and purified by silica gel column chromatography (9% MeOH in DCM) to afford tert-butyl N-ethyl-N-[1-[7-[[6-(fluoromethyl)-8-methyl-imidazo[1,2-a]pyrazin-2-yl]carbamoyl]-2-methyl-indazol-4-yl]-4-piperidyl]carbamate (22 mg, 44% yield) as solid. LCMS (ES, m/z): 565 [M+H]⁺.

### Synthesis of Compound 350

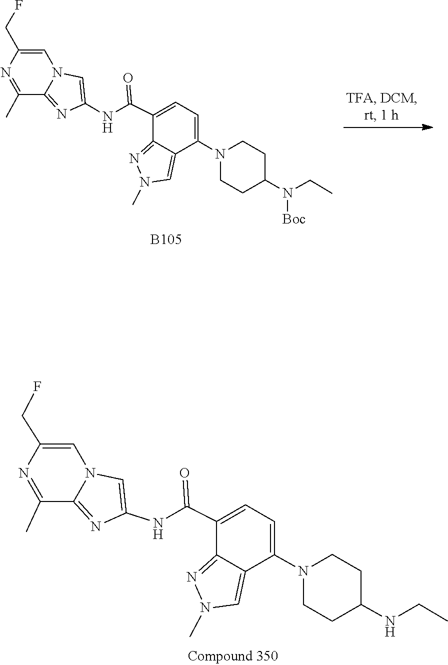 [SMILES: "B105" ; CCN(C(=O)OC(C)(C)C)C1CCN(c2ccc(C(=O)Nc3cn4cc(CF)nc(C)c4n3)c3nn(C)cc23)CC1 ; CCNC1CCN(c2ccc(C(=O)Nc3cn4cc(CF)nc(C)c4n3)c3nn(C)cc23)CC1]

To a stirred solution of tert-butyl N-ethyl-N-[1-[7-[[6-(fluoromethyl)-8-methyl-imidazo[1,2-a]pyrazin-2-yl]carbamoyl]-2-methyl-indazol-4-yl]-4-piperidyl]carbamate (20 mg, 35.42 μmol, 1 eq) in DCM (1 mL) was added TFA (0.5 mL), and the reaction was stirred for 2 h at room temperature. The resulting mixture was concentrated under reduced pressure and purified by Prep-HPLC (Condition 15, Gradient 1) to afford 4-[4-(ethylamino)-1-piperidyl]-N-[6-(fluoromethyl)-8-methyl-imidazo[1,2-a]pyrazin-2-yl]-2-methyl-indazole-7-carboxamide 2,2,2-trifluoroacetic acid (Compound 350, 8.5 mg, 41% yield) as a solid. LCMS (ES, m/z): 465 [M+H]⁺. ¹H NMR (400 MHz, DMSO-d₆) δ 11.53 (s, 1H), 8.87 (s, 1H), 8.71 (d, J=2.9 Hz, 1H), 8.47 (s, 1H), 8.45 (s, 2H), 8.07 (d, J=8.1 Hz, 1H), 6.58 (d, J=8.2 Hz, 1H), 5.43 (d, J=47.7 Hz, 2H), 4.33 (s, 3H), 4.07 (d, J=12.8 Hz, 2H), 3.12-3.01 (m, 4H), 2.76 (s, 3H), 2.18-2.09 (m, 2H), 1.71 (q, J=13.5, 13.0 Hz, 2H), 1.23 (t, J=7.2 Hz, 3H).

An analogous method was followed to obtain the following compounds.

| Compound | Starting Material | Characterization |
|---|---|---|
| 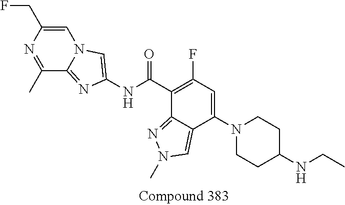 [SMILES: CCNC1CCN(c2cc(F)c(C(=O)Nc3cn4cc(CF)nc(C)c4n3)c3nn(C)cc23)CC1] | 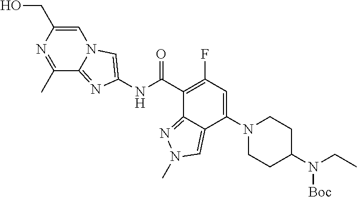 [SMILES: CCN(C(=O)OC(C)(C)C)C1CCN(c2cc(F)c(C(=O)Nc3cn4cc(CO)nc(C)c4n3)c3nn(C)cc23)CC1] | Modification: Step 2 was ran using HCl in dioxane (4 M, 0.5 mL). The crude product was purified by Prep- HPLC (Condition 2, Gradient 8) to afford 4- (4- (ethylamino)piperidin- 1-yl)-6-fluoro-N-(6- (fluoromethyl)-8- methylimidazo[1,2-a]- pyrazin-2-yl)-2- methyl-2H-indazole-7- |
| | | carboxamide 2,2,2- |
| | | trifluoroacetate |
| | | (Compound 383, 13 |
| | | mg, 21.8 μmol, 18% |
| | | yield) as a solid. |
| | | LCMS (ES, m/z): 483 |
| | | [M + H]⁺. 1H NMR |
| | | (400 MHz, DMSO-d₆) |
| | | δ 11.61 (s, 1H), 8.86 (s, |
| | | 1H), 8.68 (d, J = 3.0 |
| | | Hz, 1H), 8.47-8.41 (m, |
| | | 3H), 6.34 (d, J = 15.6 |
| | | Hz, 1H), 5.40 (d, J = |
| | | 47.7 Hz, 2H), 4.24 (s, |
| | | 3H), 4.07 (d, J = 13.1 |
| | | Hz, 2H), 3.50-3.31 (m, |
| | | 1H), 3.20-2.92 (m, |
| | | 4H), 2.72 (s, 3H), 2.10 |
| | | (d, J = 12.2 Hz, 2H), |
| | | 1.64 (q, J = 11.6, 10.4 |
| | | Hz, 2H), 1.20 (t, J = 7.2 |
| | | Hz, 3H). |

### Example 118: Synthesis of Compound 366

### Synthesis of B106

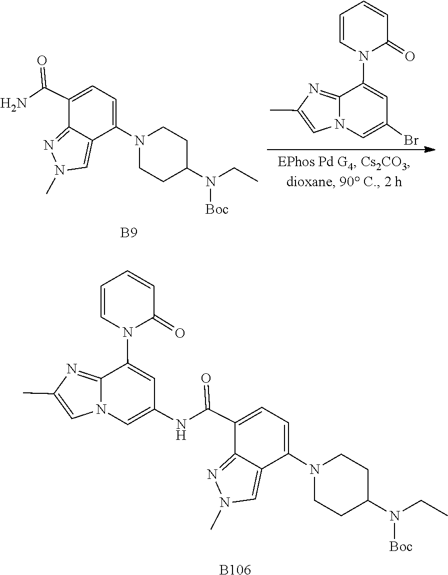

To a stirred mixture of tert-butyl N-[1-(7-carbamoyl-2-methyl-indazol-4-yl)-4-piperidyl]-N-ethyl-carbamate (120 mg, 298.9 μmol) and 1-(6-bromo-2-methyl-imidazo[1,2-a]pyridin-8-yl)pyridin-2-one (109 mg, 358.7 μmol) in Dioxane (2 mL) were added EPhos Pd G₄ (27 mg, 29.89 μmol) and Cs₂CO₃ (195 mg, 597.8 μmol) at room temperature under N₂. The resulting mixture was stirred for 2 h at 90° C. under N₂. The resulting mixture was concentrated under reduced pressure and purified by silica gel column chromatography (75% EtOAc in PE) to afford tert-butyl N-ethyl-N-[1-[2-methyl-7-[[2-methyl-8-(2-oxo-1-pyridyl)imidazo[1,2-a]pyridin-6-yl]carbamoyl]indazol-4-yl]-4-piperidyl]-carbamate (70 mg, 112 μmol, 38% yield) as a solid. LCMS (ES, m/z): 625 [M+H]⁺.

### Synthesis of Compound 366

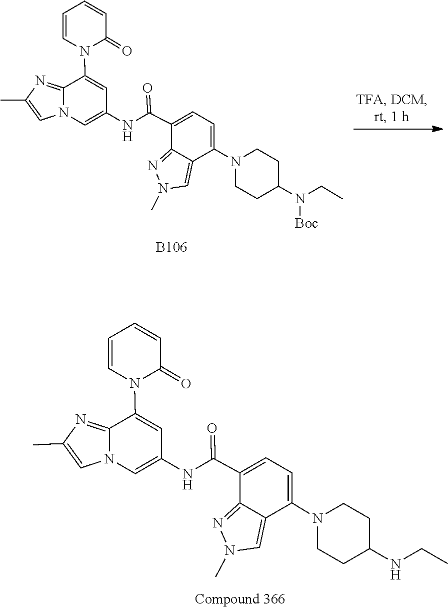

To a stirred solution of tert-butyl N-ethyl-N-[1-[2-methyl-7-[[2-methyl-8-(2-oxo-1-pyridyl)imidazo[1,2-a]pyridin-6-yl]carbamoyl]indazol-4-yl]-4-piperidyl]carbamate (70 mg, 112.1 μmol) in DCM (2 mL) was added TFA (1 mL), and the reaction was stirred for 1 h at room temperature. The resulting mixture was concentrated under reduced pressure and purified by Prep-HPLC (Condition 6, Gradient 3) to afford 4-[4-(ethylamino)-1-piperidyl]-2-methyl-N-[2-methyl-8-(2-oxo-1-pyridyl)imidazo[1,2-a]pyridin-6-yl]indazole-7-carboxamide (Compound 366, 14.8 mg, 28.21 μmol, 25% yield) as a solid. LCMS (ES, m/z): 525 [M+H]⁺. ¹H NMR (400 MHz, DMSO-d₆) δ 11.10 (s, 1H), 9.51 (d, J=1.8 Hz, 1H), 8.78 (s, 1H), 8.00 (d, J=8.1 Hz, 1H), 7.92 (d, J=1.1 Hz, 1H), 7.73 (dd, J=7.0, 2.0 Hz, 1H), 7.59 (ddd, J=9.0, 6.6, 2.1 Hz, 1H), 7.48 (d, J=1.9 Hz, 1H), 6.59-6.48 (m, 2H), 6.37 (td, J=6.7, 1.4 Hz, 1H), 4.28 (s, 3H), 3.92 (d, J=12.9 Hz, 2H), 3.06 (t, J=11.7 Hz, 2H), 2.82-2.80 (m, 1H), 2.75-2.62 (m, 2H), 2.33-2.28 (m, 3H), 2.00 (d, J=12.5 Hz, 2H), 1.57-1.39 (m, 2H), 1.08 (t, J=7.1 Hz, 3H).

An analogous method was followed to obtain the following compounds.

| Compound | Starting Material | Characterization |
|---|---|---|
| 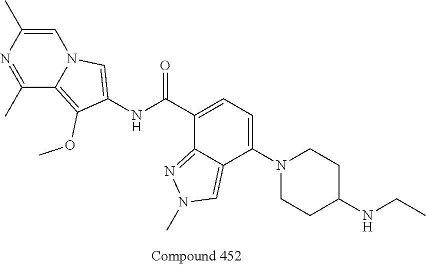 [SMILES: CCNC1CCN(c2ccc(C(=O)Nc3cn4cc(C)nc(C)c4c3OC)c3nn(C)cc23)CC1] | 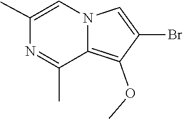 | Modification: Step 1 was ran for 3 h. Step 2 was ran for 0.5 h. The crude product was purified by Prep-HPLC (Condition 2, Gradient 9) to afford 4-(4-(ethylamino)- piperidin-1-yl)-N-(8- methoxy-1,3-dimethyl- pyrrolo[1,2-a] pyrazin-7-yl)- 2-methyl-2H-indazole-7- carboxamide 2,2,2- trifluoroacetate (Compound 452, 45 mg, 76.3 |
| | | μmol, 88% |
| | | yield) as a solid. |
| | | LCMS (ES, |
| | | m/z): 476 [M + H]⁺. |
| | | ¹H NMR |
| | | (300 MHz, DMSO-d₆) δ |
| | | 11.54 (s, 1H), 8.87 (s, 1H), |
| | | 8.74 (s, 1H), 8.58 (s, 2H), |
| | | 8.26 (s, 1H), 8.02 (d, J = 8.1 |
| | | Hz, 1H), 6.56 (d, J = 8.2 Hz, |
| | | 1H), 4.29 (s, 3H), 4.12 (s, |
| | | 3H), 4.05 (d, J = 13.1 Hz, |
| | | 2H), 3.44 (s, 1H), 3.12-2.92 |
| | | (m, 4H), 2.87 |
| | | (s, 3H), 2.34 (s, |
| | | 3H), 2.13 (d, J = 12.2 Hz, |
| | | 2H), 1.69 (q, J = 11.9 Hz, |
| | | 2H), 1.21 (t, J = 7.2 Hz, 3H). |
| 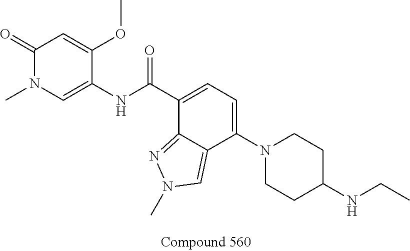 [SMILES: CCNC1CCN(c2ccc(C(=O)Nc3cn(C)c(=O)cc3OC)c3nn(C)cc23)CC1] | 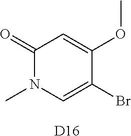 | Modification: Step 1 was ran for 16 h at 100° C. Step 2 was ran for 1 h. The crude product was purified by Prep-HPLC (Condition 5, Gradient 19b )to afford 4-[4- (ethylamino)- 1-piperidyl]-N- (4-methoxy- 1-methyl-6-oxo- 3-pyridyl)- 2-methyl-indazole-7- carboxamide (Compound 560, 30 mg, 68.4 μmol). |
| | | LCMS (ES, m/z): 439 |
| | | [M + H]⁺. ¹H NMR (400 |
| | | MHz, DMSO-d₆) δ 11.11 (s, |
| | | 1H), 8.73 (s, 2H), 7.92 (d, |
| | | J = 8.0 Hz, 1H), 6.46 (d, J = |
| | | 8.2 Hz, 1H), 5.98 (s, 1H), |
| | | 4.25 (s, 3H), 3.97 (s, 3H), |
| | | 3.86 (d, J = 12.7 Hz, 2H), |
| | | 3.41 (s, 3H), 3.15-2.96 (m, |
| | | 2H), 2.67 (dd, J = 9.2, 4.9 |
| | | Hz, 1H), 2.60 (q, J = 7.2 Hz, |
| | | 2H), 1.95 (d, J = 12.6 Hz, |
| | | 2H), 1.43 (q, J = 11.4, 9.5 |
| | | Hz, 2H), 1.04 (t, J = |
| | | 7.1 Hz, 3H). |
| 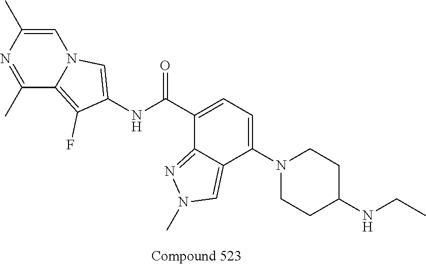 [SMILES: CCNC1CCN(c2ccc(C(=O)Nc3cn4cc(C)nc(C)c4c3F)c3nn(C)cc23)CC1] | 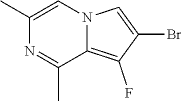 | Modification: Step 1 was ran at 110° C. for 16 h. The crude product was purified by Prep-HPLC (Condition 5, Gradient 28) to afford 4-[4- (ethylamino)-1- piperidyl]-N- (8-fluoro-1,3- dimethyl-pyrrolo[1,2- a]pyrazin-7-yl)-2-methyl- indazole-7-carboxamide (Compound 523, 20 mg, 43.2 μmol). LCMS (ES, m/z): 464 [M + H]⁺. |
| | | ¹H NMR |
| | | (300 MHz, DMSO-d₆) δ |
| | | 11.26 (s, 1H), 8.79 (s, 1H), |
| | | 8.16 (d, J = 3.7 |
| | | Hz, 1H), 7.99 |
| | | (d, J = 8.1 Hz, 1H), 7.91 (s, |
| | | 1H), 6.50 (d, J = |
| | | 8.2 Hz, 1H), |
| | | 4.26 (s, 3H), 3.89 |
| | | (d, J = 12.5 |
| | | Hz, 2H), 3.06 (t, |
| | | J = 11.3 Hz, |
| | | 2H), 2.61 (d, J = |
| | | 7.4 Hz, 6H), |
| | | 2.24 (d, J = 1.0 |
| | | Hz, 3H), 1.96 |
| | | (d, J = 12.7 Hz, |
| | | 2H), 1.44 (q, |
| | | J = 10.5, 9.8 Hz, |
| | | 2H), 1.04 (t, |
| | | J = 7.1 Hz, 3H). |
| 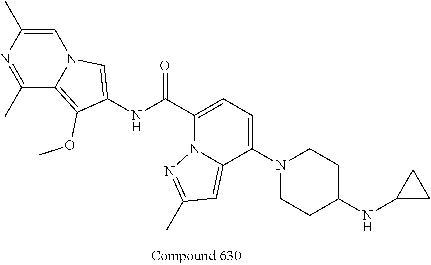 [SMILES: COc1c(NC(=O)c2ccc(N3CCC(NC4CC4)CC3)c3cc(C)nn23)cn2cc(C)nc(C)c12] | 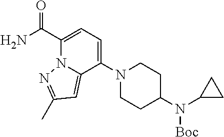 [SMILES: Cc1cc2c(N3CCC(N(C(=O)OC(C)(C)C)C4CC4)CC3)ccc(C(N)=O)n2n1] | Modification: Step 2 was ran using HCl in dioxane at room temperature for 0.5 h. The crude product was purified by Prep-HPLC (Condition 5, Gradient 6) to afford 4-[4- (cyclopropylamino)-1- piperidyl]-N- (8-methoxy- 1,3-dimethyl- pyrrolo[1,2- a]pyrazin-7- yl)-2-methyl- pyrazolo[1,5- a]pyridine-7- |
| | 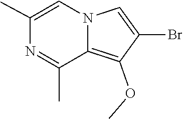 | carboxamide (Compound 630, 12 mg, 24.6 μmol, 52% yield) as a solid. LCMS (ES, m/z): 488 [M + H]⁺. ¹H NMR (400 MHz, DMSO-d₆) δ |
| | | 13.14 (s, 1H), 8.14 (s, 1H), |
| | | 7.88 (s, 1H), 7.80 (d, J = 8.1 |
| | | Hz, 1H), 6.76 (d, J = 8.2 Hz, |
| | | 1H), 6.71 (s, 1H), 4.01 (s, |
| | | 3H), 3.72 (d, J = 12.6 Hz, |
| | | 2H), 2.94 (t, J = 11.7 Hz, |
| | | 2H), 2.84-2.70 |
| | | (m, 1H), 2.66 |
| | | (s, 3H), 2.58 (s, |
| | | 3H), 2.23 (d, |
| | | J = 1.1 Hz, 3H), 2.13 (tt, J = |
| | | 6.7, 3.5 Hz, 1H), 2.02 (d, J = |
| | | 12.7 Hz, 2H), 1.51 (q, J = |
| | | 11.3, 10.7 Hz, 2H), 0.40 (dt, |
| | | J = 6.2, 3.0 Hz, 2H), 0.30- |
| | | 0.19 (m, 2H). |
| 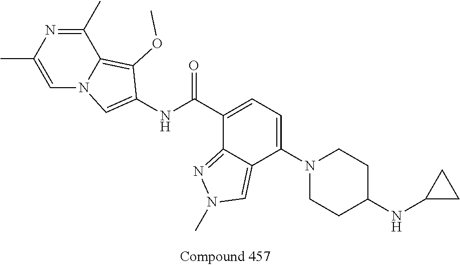 [SMILES: COc1c(NC(=O)c2ccc(N3CCC(NC4CC4)CC3)c3cn(C)nc23)cn2cc(C)nc(C)c12] | 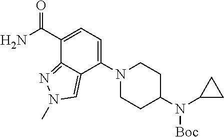 [SMILES: Cn1cc2c(N3CCC(N(C(=O)OC(C)(C)C)C4CC4)CC3)ccc(C(N)=O)c2n1] | Modification: Step 1 was ran for 3 h. Step 2 was ran for 2 h. The crude product was purified by CHIRAL-HPLC (Condition 4, Gradient 1) to afford 4-[4- (cyclopropylamino)-1- piperidyl]-N- (8-methoxy- 1,3-dimethyl-pyrrolo[1,2- a]pyrazin-7-yl)-2-methyl- indazole-7-carboxamide (Compound |
| | | 457, 4 mg, 8.2 |
| | 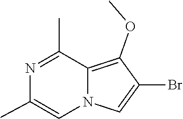 | μmol, 17% yield) as a solid. LCMS (ES, m/z): 488 [M + H]⁺. ¹H NMR (300 MHz, Methanol-d₄) δ 8.47 (s, 1H), 8.09 (s, 1H), 8.06 (d, J = 8.1 Hz, 1H), |
| | | 7.67 (s, 1H), |
| | | 6.51 (d, J = 8.2 |
| | | Hz, 1H), 4.33 |
| | | (s, 3H), 4.09 (s, |
| | | 3H), 3.99 (d, |
| | | J = 12.9 Hz, 2H), |
| | | 3.03 (t, J = |
| | | 12.4 Hz, 2H), 2.88 (s,1H), |
| | | 2.74 (s, 3H), 2.30 (s, 3H), |
| | | 2.25 (dt, J = 6.5, |
| | | 3.0 Hz, 1H), |
| | | 2.16 (d, J = 12.6 Hz, 2H), |
| | | 1.63 (q, J = 11.0 Hz, 2H), |
| | | 0.55 (q, J = 5.9, 5.4 Hz, 2H), |
| | | 0.43 (q, J = 3.6, 2.9 Hz, 2H). |
| 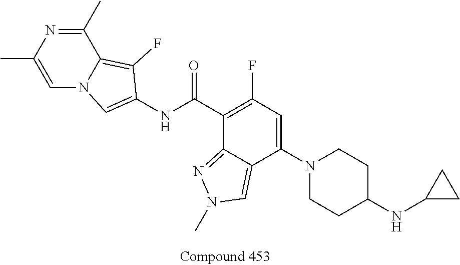 [SMILES: Cc1cn2cc(NC(=O)c3c(F)cc(N4CCC(NC5CC5)CC4)c4cn(C)nc34)c(F)c2c(C)n1] | 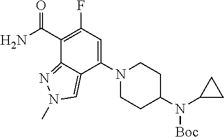 [SMILES: Cn1cc2c(N3CCC(N(C(=O)OC(C)(C)C)C4CC4)CC3)cc(F)c(C(N)=O)c2n1] | Modification: Step 1 was ran for 6 h at 100° C. Step 2 was ran for 2 h. The crude product was purified by Prep-HPLC (Condition 5, Gradient 15) to afford 4-[4- (cyclopropylamino)-1- piperidyl]-6-fluoro-N-(8- fluoro-1,3-dimethyl- pyrrolo[1,2-a] pyrazin-7-yl)- 2-methyl-indazole-7- carboxamide (Compound 453, 22 mg, 44.6 |
| | | μmol, 42% |
| | 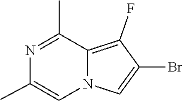 | yield) as a solid. LCMS (ES, m/z): 494 [M + H]⁺. ¹H NMR (400 MHz, DMSO-d₆) δ 11.37 (s, 1H), 8.82 (s, 1H), 8.12 (d, J = 3.6 |
| | | Hz, 1H), 7.89 |
| | | (s, 1H), 6.24 (d, |
| | | J = 15.9 Hz, |
| | | 1H), 4.20 (s, 3H), 3.91 |
| | | (d, J = 13.1 Hz, |
| | | 2H), 3.13 (t, J = |
| | | 11.9 Hz, 2H), |
| | | 2.89-2.74 (m, |
| | | 1H), 2.62 (s, |
| | | 3H), 2.24 (s, |
| | | 3H), 2.12 (tt, J = |
| | | 6.7, 3.6 Hz, |
| | | 1H), 1.99 (d, J = 12.7 Hz, |
| | | 2H), 1.45 (q, J = 11.5 Hz, |
| | | 2H), 0.40 (dt, J = |
| | | 6.3, 3.0 Hz, |
| | | 2H), 0.23 (p, J = |
| | | 4.0 Hz, 2H). |
| 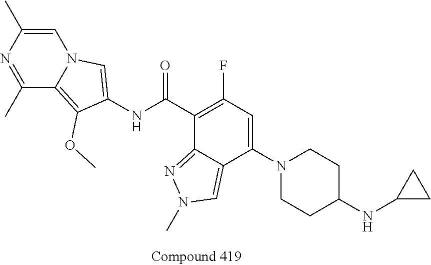 [SMILES: COc1c(NC(=O)c2c(F)cc(N3CCC(NC4CC4)CC3)c3cn(C)nc23)cn2cc(C)nc(C)c12] | 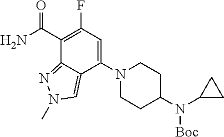 [SMILES: Cn1cc2c(N3CCC(N(C(=O)OC(C)(C)C)C4CC4)CC3)cc(F)c(C(N)=O)c2n1] | Modification: Step 1 was ran for 5 h at 90° C. Step 2 was ran for 2 h. The crude product was purified by Prep-HPLC (Condition 5, Gradient 23) to afford 4-[4- (cyclopropylamino)-1- piperidyl]-6-fluoro-N-(8- methoxy-1,3-dimethyl- pyrrolo[1,2-a] pyrazin-7-yl)- 2-methyl-indazole-7- carboxamide (Compound 419, 31.9 mg, 63.1 μmol, 43% yield) as a |
| | | solid. LCMS |
| | 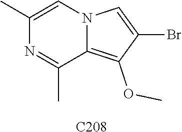 | (ES, m/z): 506 [M + H]⁺. ¹H NMR (400 MHz, DMSO-d₆) δ 11.43 (s, 1H), 8.83 (s, 1H), 8.10 (s, 1H), 7.84 (s, 1H), 6.23 (d, J = 16.0 Hz, 1H), 4.24 (s, 3H), 4.00 (s, 3H), |
| | | 3.91 (d, J = 12.7 Hz, 2H), |
| | | 3.18-3.07 (m, 2H), 2.80 (td, |
| | | J = 9.6, 4.7 Hz, |
| | | 1H), 2.64 (s, |
| | | 3H), 2.30 (s, 1H), 2.22 (s, |
| | | 3H), 2.11 (tt, J = |
| | | 6.7, 3.6 Hz, |
| | | 1H), 1.99 (d, J = 11.8 Hz, |
| | | 2H), 1.45 (q, J = 11.5, 9.8 |
| | | Hz, 2H), 0.39 (dt, |
| | | J = 6.2, 3.0 Hz, |
| | | 2H), 0.27-0.19 (m, 2H). |
| 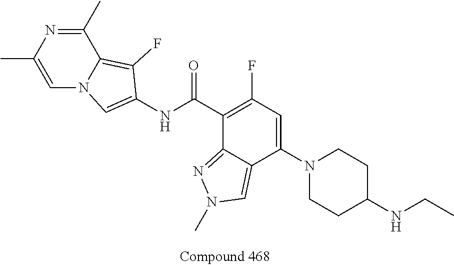 [SMILES: CCNC1CCN(c2cc(F)c(C(=O)Nc3cn4cc(C)nc(C)c4c3F)c3nn(C)cc23)CC1] | 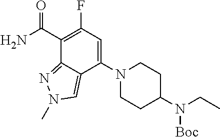 [SMILES: CCN(C(=O)OC(C)(C)C)C1CCN(c2cc(F)c(C(N)=O)c3nn(C)cc23)CC1] | Modification: Step 1 was ran for 4 h. Step 2 was ran using HCl in dioxane at room temperature for 2 h. The crude product was purified by Prep-HPLC (Condition 5, Gradient 25) to afford 4-[4- (ethylamino)-1- piperidyl]-6-fluoro-N-(8- fluoro-1,3-dimethyl- pyrrolo[1,2-a] pyrazin-7-yl)- |
| | | 2-methyl-indazole-7- |
| | 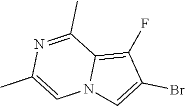 | carboxamide (Compound 468, 6.3 mg, 13.1 μmol). LCMS (ES, m/z): 482 [M + H]⁺. ¹H NMR (400 MHz, Methanol- d₄) δ 8.52 (s, 1H), 8.07 (d, J = |
| | | 3.5 Hz, 1H), |
| | | 7.70 (s, 1H), 6.25 |
| | | (d, J = 16.0 |
| | | Hz, 1H), 4.25 (s, 3H), 4.04 |
| | | (d, J = 12.9 Hz, |
| | | 2H), 3.08 (d, |
| | | J = 12.3 Hz, 1H), 2.84 (s, |
| | | 1H), 2.76 (q, J = |
| | | 7.2 Hz, 2H), |
| | | 2.68 (s, 3H), 2.30 (d, J = |
| | | 1.1 Hz, 3H), 2.10 |
| | | (d, J = 12.5 Hz, |
| | | 2H), 1.59 (s, 1H), 1.55 (d, |
| | | J = 10.1 Hz, 1H), 1.18 (t, |
| | | J = 7.2 Hz, 3H). |
| 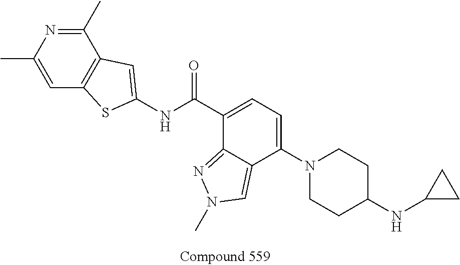 [SMILES: Cc1cc2sc(NC(=O)c3ccc(N4CCC(NC5CC5)CC4)c4cn(C)nc34)cc2c(C)n1] | 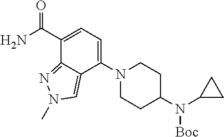 | Modification: Step 1 was ran for 6 h at 100° C. Step 2 was ran for 1 h. The crude product was purified by Prep-HPLC (Condition 5, Gradient 28) to afford 4-[4- (cyclopropylamino)-1- piperidyl]-N- (4,6-dimethyl- thieno[3,2-c]- pyridin-2-yl)- 2-methyl-indazole-7- carboxamide (Compound 559, 25 mg, 52.7 μmol). LCMS (ES, m/z): 475 |
| | | [M + H]⁺. ¹H NMR (400 |
| | 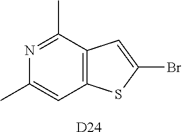 | MHz, DMSO-d₆) δ 12.03 (s, 1H), 8.83 (s, 1H), 7.99 (d, J = 8.2 Hz, 1H), 7.58 (s, 1H), 7.26 (s, 1H), 6.51 (d, J = 8.3 Hz, 1H), 4.32 (s, 3H), 3.93 (d, J = 12.9 Hz, |
| | | 2H), 3.12 (t, |
| | | J = 11.6 Hz, 2H), 2.80 |
| | | (dt, J = 9.9, 5.6 Hz, |
| | | 1H), 2.69 (s, |
| | | 3H), 2.50 (s, |
| | | 3H), 2.32 (d, J = |
| | | 15.1 Hz, 1H), 2.13 (tt, J = |
| | | 6.8, 3.6 Hz, 1H), 2.01 (d, |
| | | J = 12.7 Hz, 2H), |
| | | 1.47 (q, J = 9.9 |
| | | Hz, 2H), 0.46-0.33 (m, 2H), |
| | | 0.31-0.16 (m, 2H). |
| 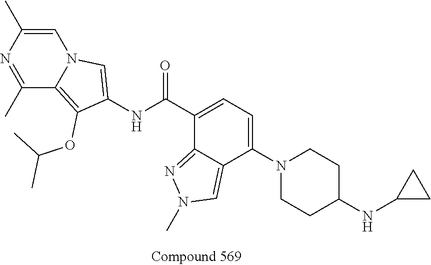 [SMILES: Cc1cn2cc(NC(=O)c3ccc(N4CCC(NC5CC5)CC4)c4cn(C)nc34)c(OC(C)C)c2c(C)n1] | 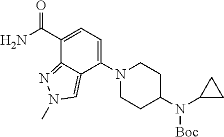 | Modification: Step 1 was ran for 2 h at 90° C. The crude product was purified by Prep-HPLC (Condition 5, Gradient 12a) to afford 4-[4- (cyclopropylamino)-1- piperidyl]-N- (8-isopropoxy- 1,3-dimethyl-pyrrolo[1,2- a]pyrazin-7-yl)-2-methyl- indazole-7-carboxamide (Compound 569, 13 mg, 25.2 μmol). LCMS (ES, m/z): 516 [M + H]⁺. ¹H NMR |
| | | (400 MHz, DMSO-d₆) δ |
| | 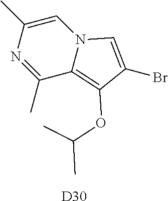 | 11.04 (s, 1H), 8.76 (s, 1H), 8.17 (s, 1H), 7.97 (d, J = 8.1 Hz, 1H), 7.83 (s, 1H), 6.47 (d, J = 8.2 Hz, 1H), 4.39 (p, J = 6.1 Hz, 1H), 4.27 (s, 3H), 3.85 (d, J = 12.7 Hz, 2H), 3.03 (t, J = 11.6 Hz, 2H), 2.76 (ddd, J = 13.4, 9.3, 3.6 Hz, 1H), 2.62 (s, 3H), 2.20 (s, |
| | | 3H), 2.10 (tt, J = |
| | | 6.7, 3.6 Hz, |
| | | 1H), 1.98 (d, J = 12.5 Hz, |
| | | 2H), 1.53-1.34 |
| | | (m, 8H), 0.38 |
| | | (td, J = 6.3, 4.0 Hz, 2H), |
| | | 0.21 (p, J = 4.0 Hz, 2H). |
| 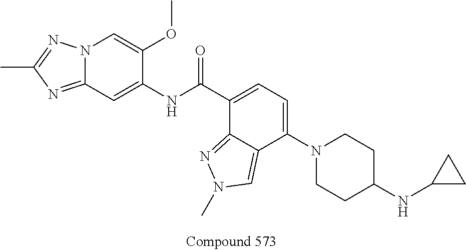 | 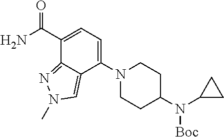 | Modification: Step 1 was ran for 2 h at 100° C. The crude product was purified by Prep-HPLC (Condition 5, Gradient 33) to afford 4-[4- (cyclopropylamino)-1- piperidyl]-N- (6-methoxy-2- methyl-[1,2,4] triazolo-[1,5-a]- pyridin-7-yl)-2-methyl- indazole-7-carboxamide (Compound 573, 21 mg, 44.3 μmol). LCMS (ES, |
| | | m/z): 475 [M + H]⁺. |
| | 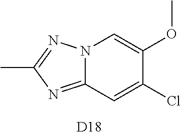 | ¹H NMR (400 MHz, DMSO-d₆) δ 11.90 (s, 1H), 8.78 (s, 1H), 8.72 (s, 1H), 8.62 (s, 1H), 8.02 (d, J = 8.2 Hz, 1H), 6.49 (d, J = 8.3 Hz, 1H), 4.28 (s, 3H), 4.09 (s, 3H), 3.91 (d, J = 13.0 Hz, 2H), 3.15-3.04 |
| | | (m, 2H), 2.79 (s, |
| | | 1H), 2.41 (s, |
| | | 3H), 2.25 (s, |
| | | 1H), 2.12 (dt, |
| | | J = 6.6, 3.2 Hz, |
| | | 1H), 2.00 (d, |
| | | J = 12.5 Hz, 2H), 1.57-1.33 |
| | | (m, 2H), 0.40 (dt, |
| | | J = 6.2, 3.0 |
| | | Hz, 2H), 0.27-0.19 (m, 2H). |
| 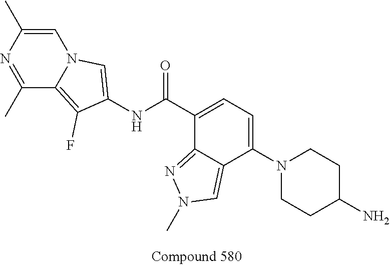 [SMILES: Cc1cn2cc(NC(=O)c3ccc(N4CCC(N)CC4)c4cn(C)nc34)c(F)c2c(C)n1] | 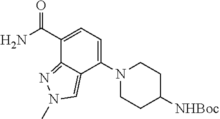 | Modification: Step 1 was ran for 16 h at 100° C. Step 2 was ran for 1 h. The crude product was purified by Prep-HPLC (Condition 5, Gradient 30) to afford 4-(4-amino-1- piperidyl)-N- (8-fluoro-1,3- dimethyl-pyrrolo[1,2-a]- pyrazin-7-yl)-2-methyl- indazole-7-carboxamide (Compound 580, 14 mg, 32.2 μmol). LCMS (ES, m/z): 436 [M + H]⁺. |
| | | ¹H NMR |
| | 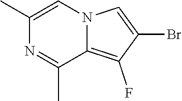 | (400 MHz, DMSO-d₆) δ 11.26 (s, 1H), 8.78 (s, 1H), 8.15 (d, J = 3.6 Hz, 1H), 7.99 (d, J = 8.1 Hz, 1H), 7.90 (s, 1H), 6.49 (d, J = 8.3 Hz, 1H), |
| | | 4.26 (s, 3H), 3.89 |
| | | (d, J = 12.9 |
| | | Hz, 2H), 3.05 |
| | | (t, J = 11.8 Hz, |
| | | 2H), 2.85 (dt, J = 10.0, |
| | | 5.6 Hz, 1H), 2.62 |
| | | (d, J = 1.3 Hz, |
| | | 3H), 2.24 (d, J = |
| | | 1.0 Hz, 3H), |
| | | 1.87 (d, J = 12.9 Hz, 2H), |
| | | 1.50-1.37 (m, 2H). |

### Example 119: Synthesis of C94

### Synthesis of C91

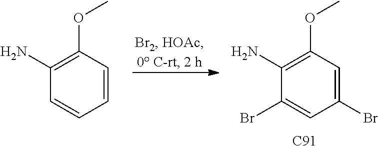

To a mixture of 2-methoxyaniline (5 g, 40.6 mmol) in AcOH (50 mL) was added Br₂ (12.98 g, 81.2 mmol) at 0° C. The mixture was stirred for 2 h at 0° C. under N₂. The precipitated solids were collected by filtration and washed with AcOH (10 mL), to afford 2,4-dibromo-6-methoxy-aniline (2.5 g, 8.90 mmol, 22% yield) as a solid. LCMS (ES, m/z): 280 [M+H]⁺.

### Synthesis of C92

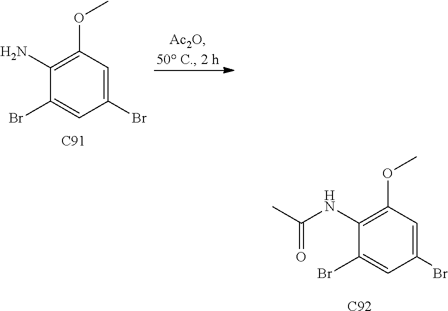

A mixture of 2,4-dibromo-6-methoxy-aniline (2.5 g, 8.90 mmol) in Acetic anhydride (30 mL) was stirred for 2 h at 50° C. under N₂. The resulting mixture was allowed to cool to room temperature, diluted with water (50 mL), and extracted with EtOAc (3×50 mL). The combined organic layers were washed with brine (2×20 mL), dried over anhydrous Na₂SO₄, and filtered. The filtrate was concentrated under reduced pressure and purified by silica gel column chromatography (75% EtOAc in PE) to afford N-(2,4-dibromo-6-methoxy-phenyl) acetamide (2.6 g, 8.05 mmol, 90% yield) as a solid. LCMS (ES, m/z): 323 [M+H]⁺.

### Synthesis of C93

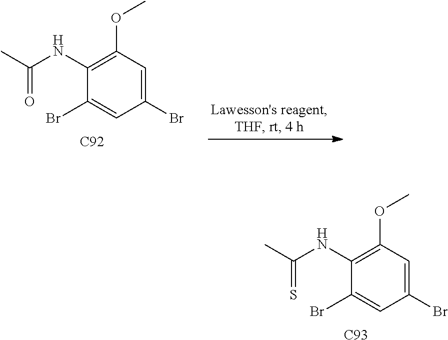

To a stirred mixture of N-(2,4-dibromo-6-methoxy-phenyl) acetamide (2.5 g, 7.74 mmol) in THF (30 mL) was added Lawesson s Reagent (6.25 g, 15.5 mmol) dropwise, and the reaction was stirred for additional 4 h at rt. The resulting mixture was diluted with water (100 mL) and extracted with EtOAc (3×60 mL). The combined organic layers were washed with brine (2×50 mL), dried over anhydrous Na₂SO₄, and filtered. The filtrate was concentrated under reduced pressure and purified by silica gel column chromatography (67% EtOAc in PE) to afford N-(2,4-dibromo-6-methoxy-phenyl)thioacetamide (1.9 g, 5.60 mmol, 72% yield) as a solid. LCMS (ES, m/z): 339 [M+H]⁺.

### Synthesis of C94

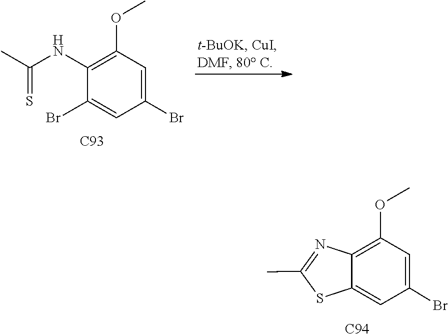

To a stirred mixture of N-(2,4-dibromo-6-methoxy-phenyl)thioacetamide (1.9 g, 5.60 mmol) in DMF (20 mL) were added t-BuOK (1.26 g, 11.21 mmol), CuI (213 mg, 1.12 mmol), and the reaction was stirred for 2 h at 80° C. under N₂. The mixture was allowed to cool to room temperature, diluted with water (50 mL), and extracted with EtOAc (3×40 mL). The combined organic layers were washed with water (2×20 mL), brine (40 mL), dried over anhydrous Na₂SO₄, and filtered. The filtrate was concentrated under reduced pressure and purified by silica gel column chromatography (75% EtOAc in PE) to afford 6-bromo-4-methoxy-2-methyl-1,3-benzothiazole (850 mg, 3.29 mmol, 59% yield) as a solid. LCMS (ES, m/z): 258 [M+H]⁺.

### Example 120: Synthesis of Compound 317

### Synthesis of B107

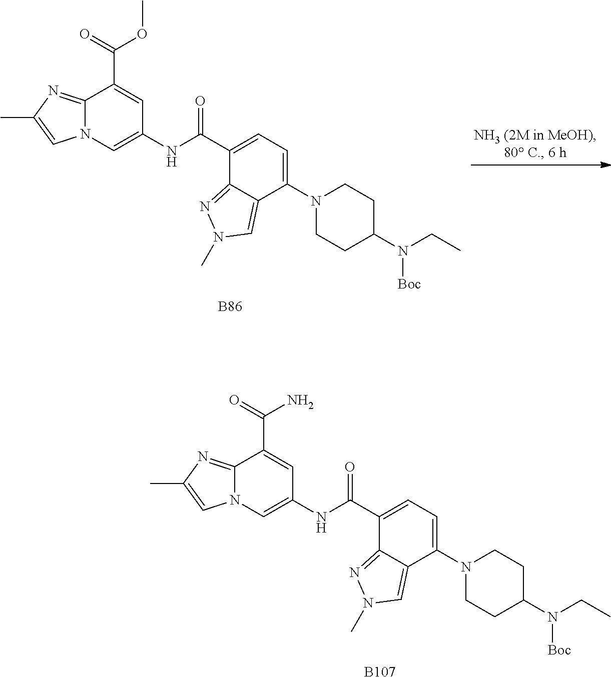

A mixture of methyl 6-[[4-[4-[tert-butoxycarbonyl(ethyl)amino]-1-piperidyl]-2-methyl-indazole-7-carbonyl]amino]-2-methyl-imidazo[1,2-a]pyridine-8-carboxylate (50 mg, 84.79 μmol) in NH₃ (2.0 M in MeOH), (1 mL) was stirred for 6 h at 80° C. The resulting mixture was concentrated under reduced pressure to afford tert-butyl N-[1-[7-[(8-carbamoyl-2-methyl-imidazo[1,2-a]pyridin-6-yl)carbamoyl]-2-methyl-indazol-4-yl]-4-piperidyl]-N-ethyl-carbamate (45 mg, 78.31 μmol, 92% yield) as a solid. LCMS (ES, m/z): 575 [M+H]⁺.

### Synthesis of Compound 317

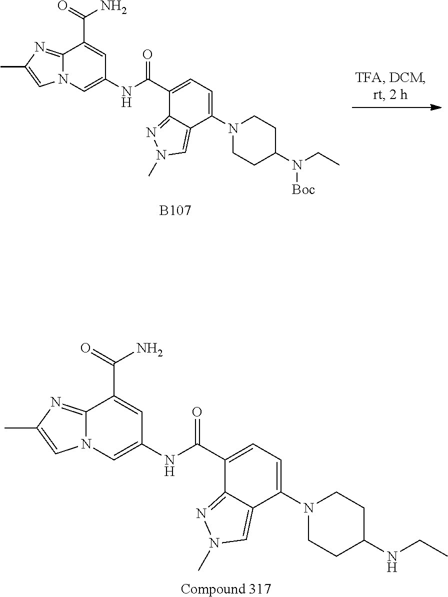

To a solution of tert-butyl N-[1-[7-[(8-carbamoyl-2-methyl-imidazo[1,2-a]pyridin-6-yl)carbamoyl]-2-methyl-indazol-4-yl]-4-piperidyl]-N-ethyl-carbamate (45 mg, 78.31 μmol) in DCM (1 mL) was added TFA (0.5 mL), and the reaction was stirred for 2 h at rt. The resulting mixture was concentrated under reduced pressure and purified by reversed-phase flash chromatography (Condition 4, Gradient 3) to afford N-(8-carbamoyl-2-methylimidazo[1,2-a]pyridin-6-yl)-4-(4-(ethylamino)piperidin-1-yl)-2-methyl-2H-indazole-7-carboxamide 2,2,2-trifluoroacetate (10.2 mg, 17.33 μmol, 22% yield) as a solid. LCMS (ES, m/z): 475 [M+H-TFA]⁺. ¹H NMR (400 MHz, DMSO-d₆) δ 13.78 (s, 1H), 11.30 (s, 1H), 9.77 (s, 1H), 8.90 (s, 1H), 8.56 (s, 2H), 8.19-8.15 (m, 2H), 8.04 (d, J=8.1 Hz, 1H), 6.58 (d, J=8.2 Hz, 1H), 4.34 (s, 3H), 4.07 (d, J=13.0 Hz, 2H), 3.37 (s, 1H), 3.06 (t, J=12.0 Hz, 4H), 2.47 (s, 3H), 2.15 (d, J=12.0 Hz, 2H), 1.78-1.65 (m, 2H), 1.24 (t, J=7.2 Hz, 3H).

An analogous method was followed to obtain the following compounds.

| | Starting | |
|---|---|---|
| Compound | Material | Characterization |
| 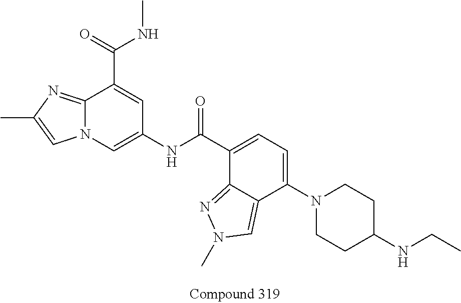 [SMILES: CCNC1CCN(c2ccc(C(=O)Nc3cc(C(=O)NC)c4nc(C)cn4c3)c3nn(C)cc23)CC1] | MeNH₂ (2 M) in MeOH | The residue was purified by reversed-phase flash chromatography (Condition 4, Gradient 3) to afford 4-(4-(ethylamino)-piperidin-1- yl)-2-methyl-N-(2-methyl-8- (methylcarbamoyl)-imidazo[1,2-a]-pyridin- 6-yl)-2H-indazole-7-carboxamide 2,2,2- trifluoroacetate (Compound 319, 18.3 mg, 30.4 μmol, 45% yield) as a solid. LCMS (ES, m/z): 489 [M + H − TFA]⁺. ¹H NMR (400 MHz, DMSO-d₆) δ 11.28 (s, 1H), 9.73 (s, 2H), 8.90 (s, 1H), 8.56 (s, 2H), 8.21 (s, 1H), 8.14 (s, 1H), 8.04 (d, J = 8.1 Hz, 1H), 6.58 (d, J = 8.2 Hz, 1H), 4.33 (s, 3H), 4.07 (d, J = 12.8 Hz, 2H), 3.36 (s, 1H), 3.06 (t, J = 11.7 Hz, 4H), 2.97 (d, J = 4.6 Hz, 3H), 2.47 (s, 3H), 2.15 (d, J = 12.2 Hz, 2H), 1.71 (q, J = 11.9 Hz, 2H), 1.23 (t, J = 7.2 Hz, 3H). |

### Example 121: Synthesis of Compound 318

### Synthesis of B108

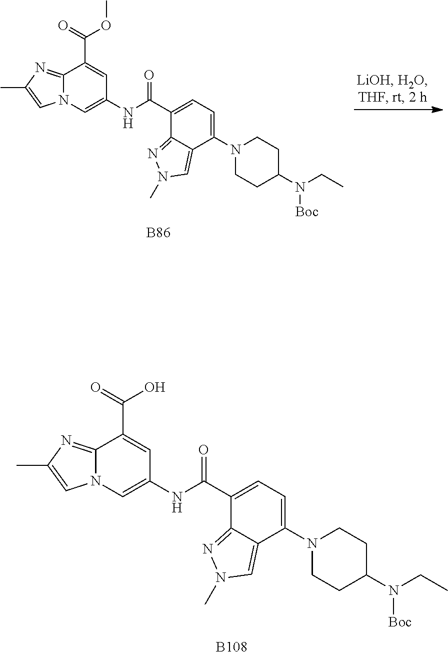

To a stirred mixture of methyl 6-[[4-[4-[tert-butoxycarbonyl(ethyl)amino]-1-piperidyl]-2-methyl-indazole-7-carbonyl]amino]-2-methyl-imidazo[1,2-a]pyridine-8-carboxylate (50 mg, 84.79 μmol) in H₂O (1 mL) and THF (1 mL) was added LiOH (10.2 mg, 424 μmol), and the reaction was stirred for 2 h at room temperature. The resulting mixture was acidified to pH 5 with HCl (1 M) and extracted with DCM (3×5 mL). The combined organic layers were washed with brine (10 mL), dried over anhydrous Na₂SO₄, and filtered. The filtrate was concentrated under reduced pressure, to afford 6-[[4-[4-[tert-butoxycarbonyl(ethyl)amino]-1-piperidyl]-2-methyl-indazole-7-carbonyl]amino]-2-methyl-imidazo[1,2-a]pyridine-8-carboxylic acid (45 mg, 78.17 μmol, 92% yield) as a solid. LCMS (ES, m/z): 576 [M+H]⁺.

### Synthesis of Compound 318

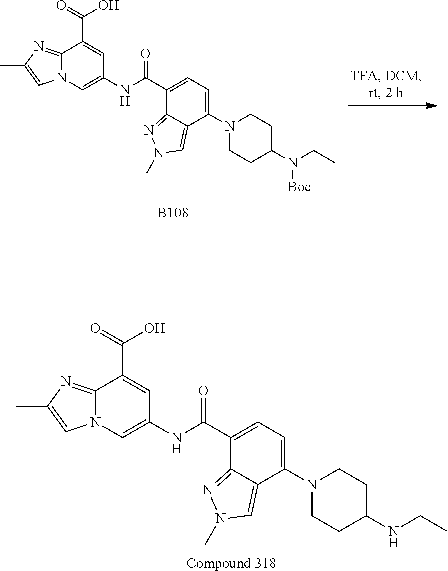

To a solution of 6-[[4-[4-[tert-butoxycarbonyl(ethyl)amino]-1-piperidyl]-2-methyl-indazole-7-carbonyl]amino]-2-methyl-imidazo[1,2-a]pyridine-8-carboxylic acid (45 mg, 78.17 μmol) in DCM (1 mL) was added TFA (0.5 mL), and the reaction was stirred for 2 h at rt. The resulting mixture was concentrated under reduced pressure and purified by reversed-phase flash chromatography (Condition 4, Gradient 3) to afford 6-[[4-[4-(ethylamino)-1-piperidyl]-2-methyl-indazole-7-carbonyl]amino]-2-methyl-imidazo[1,2-a]pyridine-8-carboxylic acid 2,2,2-trifluoroacetic acid (14.9 mg, 25.27 μmol, 32% yield) as a solid. LCMS (ES, m/z): 476 [M+H-TFA]⁺. ¹H NMR (400 MHz, DMSO-d₆) δ 11.32 (s, 1H), 9.87 (d, J=2.0 Hz, 1H), 8.88 (s, 1H), 8.57 (s, 2H), 8.45-8.40 (m, 1H), 8.26 (s, 1H), 8.03 (d, J=8.1 Hz, 1H), 6.58 (d, J=8.2 Hz, 1H), 4.32 (s, 3H), 4.07 (d, J=13.0 Hz, 2H), 3.12-3.01 (m, 4H), 2.15 (d, J=12.3 Hz, 2H), 1.72 (t, J=11.6 Hz, 2H), 1.23 (t, J=7.2 Hz, 3H).

### Example 122: Synthesis of C97

### Synthesis of C95

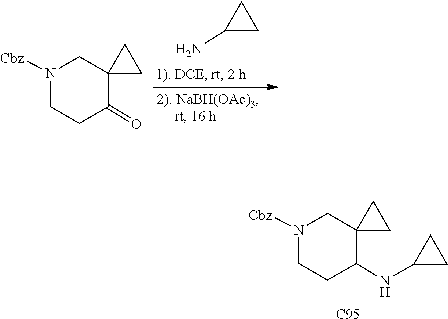

To a stirred solution of benzyl 8-oxo-5-azaspiro[2.5]octane-5-carboxylate (500 mg, 1.93 mmol) in DCE (10 mL) were added cyclopropanamine (450 mg, 7.88 mmol, 4.0 eq), and the reaction was stirred for 2 h at rt. To the above mixture was added NaBH(OAc)₃ (1.25 g, 5.90 mmol, 3.0 eq), and the reaction was stirred for additional 16 h at rt. The resulting mixture was diluted with H₂O (20 mL), basified to pH 8 with sat. Na₂CO₃ (aq), and extracted with DCM (3×20 mL). The combined organic layers were washed with brine (30 mL), dried over anhydrous Na₂SO₄, and filtered. The filtrate was concentrated under reduced pressure to afford benzyl 8-(cyclopropylamino)-5-azaspiro[2.5]octane-5-carboxylate (640 mg, crude) as a solid. LCMS (ES, m/z): 301 [M+H]⁺.

### Synthesis of C96

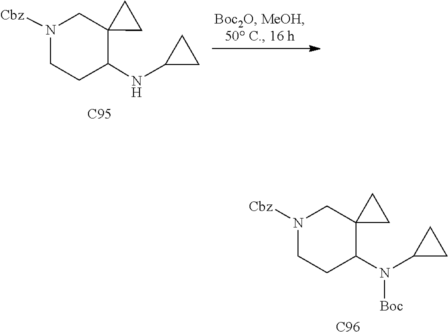 [SMILES: O=C(OCc1ccccc1)N1CCC(NC2CC2)C2(CC2)C1 ; "C95" ; CC(C)(C)OC(=O)N(C1CC1)C1CCN(C(=O)OCc2ccccc2)CC12CC2 ; "C96"]

To a stirred solution of benzyl 8-(cyclopropylamino)-5-azaspiro[2.5]octane-5-carboxylate (620 mg, 2.06 mmol, 1.0 eq) in MeOH (10 mL) was added Boc₂O (2.25 g, 10.32 mmol, 5.0 eq) at rt. The resulting mixture was stirred for 16 h at 50° C. The resulting mixture was concentrated under reduced pressure and purified by silica gel column chromatography (50% THF in PE) to afford benzyl 8-[tert-butoxycarbonyl(cyclopropyl)amino]-5-azaspiro[2.5]octane-5-carboxylate (650 mg, 1.62 mmol, 79% yield) as an oil. LCMS (ES, m/z): 401 [M+H]⁺.

### Synthesis of C97

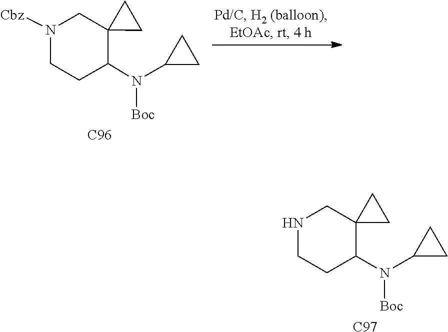 [SMILES: CC(C)(C)OC(=O)N(C1CC1)C1CCN(C(=O)OCc2ccccc2)CC12CC2 ; "C96" ; CC(C)(C)OC(=O)N(C1CC1)C1CCNCC12CC2 ; "C97"]

To a solution of benzyl 8-[tert-butoxycarbonyl(cyclopropyl)amino]-5-azaspiro[2.5]octane-5-carboxylate (640 mg, 1.60 mmol) in 30 mL of EtOAc was added Pd/C (10%, 320 mg) under N₂ in a 100 mL round-bottom flask. The mixture was hydrogenated at room temperature for 4 h under hydrogen atmosphere using a hydrogen balloon, filtered through a Celite pad and concentrated under reduced pressure to afford tert-butyl N-(5-azaspiro[2.5]octan-8-yl)-N-cyclopropyl-carbamate (390 mg, 1.46 mmol, 92% yield) as a solid. LCMS (ES, m/z): 267 [M+H]⁺.

### Example 123: Separation of Compounds 367 and 368

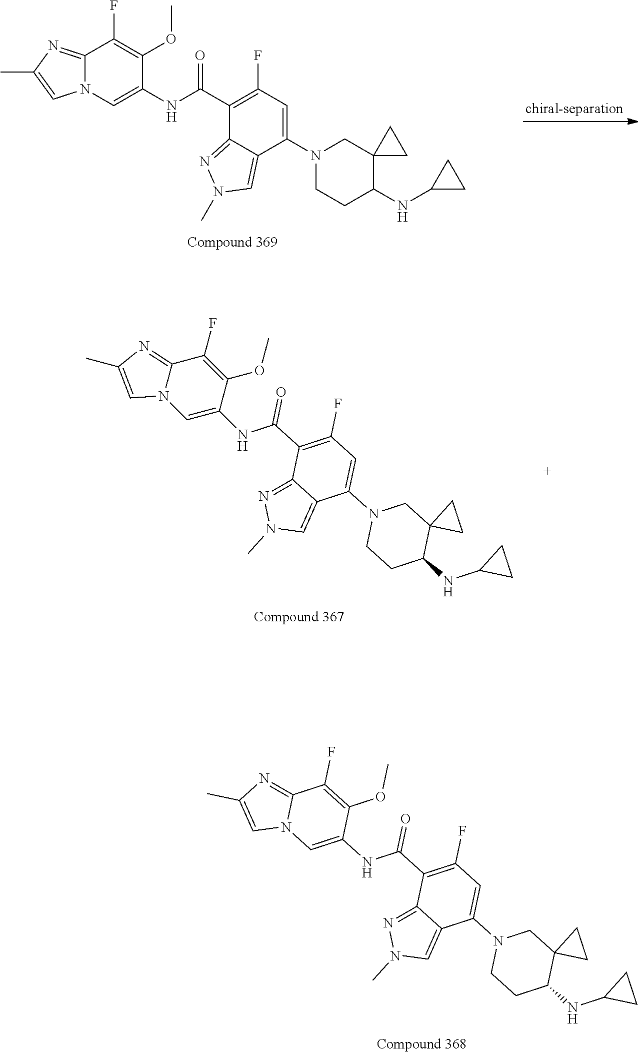 [SMILES: COc1c(NC(=O)c2c(F)cc(N3CCC(NC4CC4)C4(CC4)C3)c3cn(C)nc23)cn2cc(C)nc2c1F ; COc1c(NC(=O)c2c(F)cc(N3CC[C@H](NC4CC4)C4(CC4)C3)c3cn(C)nc23)cn2cc(C)nc2c1F ; COc1c(NC(=O)c2c(F)cc(N3CC[C@@H](NC4CC4)C4(CC4)C3)c3cn(C)nc23)cn2cc(C)nc2c1F]

### Compound 367:

The racemic products (30 mg) was separated by Chiral-HPLC (Condition 3, Gradient 1) to afford 4-[(8S)-8-(cyclopropylamino)-5-azaspiro[2.5]octan-5-yl]-6-fluoro-N-(8-fluoro-7-methoxy-2-methyl-imidazo[1,2-a]pyridin-6-yl)-2-methyl-indazole-7-carboxamide (9 mg, 16.8 μmol, 36% yield) as a solid. LCMS (ES, m/z): 536 [M+H]⁺. Chiral-HPLC (time): 1.57 min. 1H NMR (400 MHz, DMSO-d₆) δ 11.56 (s, 1H), 9.46 (d, J=1.1 Hz, 1H), 8.75 (s, 1H), 7.76 (d, J=3.0 Hz, 1H), 6.20 (d, J=16.4 Hz, 1H), 4.23 (s, 3H), 4.18 (d, J=1.9 Hz, 3H), 3.68-3.53 (m, 3H), 3.08 (d, J=12.7 Hz, 1H), 2.35 (d, J=4.3 Hz, 1H), 2.32-2.28 (m, 3H), 2.11-2.00 (m, 2H), 1.89-1.80 (m, 1H), 0.57 (dd, J=8.5, 4.4 Hz, 1H), 0.41 (dtd, J=18.2, 9.3, 5.1 Hz, 5H), 0.33-0.17 (m, 2H).

### Compound 368:

The racemic products (30 mg) was separated by Chiral-HPLC (Condition 3, Gradient 1) to afford 4-[(8R)-8-(cyclopropylamino)-5-azaspiro[2.5]octan-5-yl]-6-fluoro-N-(8-fluoro-7-methoxy-2-methyl-imidazo[1,2-a]pyridin-6-yl)-2-methyl-indazole-7-carboxamide (10 mg, 18.7 μmol, 40% yield, assumed) as a solid. LCMS (ES, m/z): 536 [M+H]⁻. Chiral-HPLC (time): 1.85 min. ¹H NMR (400 MHz, DMSO-d₆) δ 11.56 (s, 1H), 9.46 (d, J=1.1 Hz, 1H), 8.75 (s, 1H), 7.76 (d, J=3.0 Hz, 1H), 6.20 (d, J=16.4 Hz, 1H), 4.23 (s, 3H), 4.18 (d, J=1.9 Hz, 3H), 3.68-3.53 (m, 3H), 3.08 (d, J=12.7 Hz, 1H), 2.35 (d, J=4.3 Hz, 1H), 2.32-2.28 (m, 3H), 2.11-2.00 (m, 2H), 1.89-1.80 (m, 1H), 0.57 (dd, J=8.5, 4.4 Hz, 1H), 0.41 (dtd, J=18.2, 9.3, 5.1 Hz, 5H), 0.33-0.17 (m, 2H).

### Example 124: Synthesis of Compound 335

### Synthesis of B109

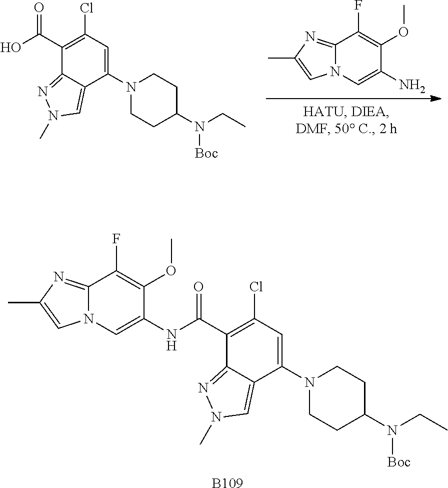

To a stirred solution of 4-[4-[tert-butoxycarbonyl(ethyl)amino]-1-piperidyl]-6-chloro-2-methyl-indazole-7-carboxylic acid (120 mg, 274.6 μmol) and 8-fluoro-7-methoxy-2-methyl-imidazo[1,2-a]pyridin-6-amine (81 mg, 412 μmol) in DMF (4 mL) were added HATU (157 mg, 412 μmol) and DIEA (107 mg, 823.9 μmol), and the reaction was stirred for 2 h at 50° C. The resulting mixture was diluted with water (20 mL). The precipitated solids were collected by filtration and washed with water (5 mL), to afford tert-butyl N-[1-[6-chloro-7-[(8-fluoro-7-methoxy-2-methyl-imidazo[1,2-a]pyridin-6-yl)carbamoyl]-2-methyl-indazol-4-yl]-4-piperidyl]-N-ethyl-carbamate (90 mg, 146.6 μmol, 53% yield) as a solid. LCMS (ES, m/z): 614 [M+H]⁺.

### Synthesis of Compound 335

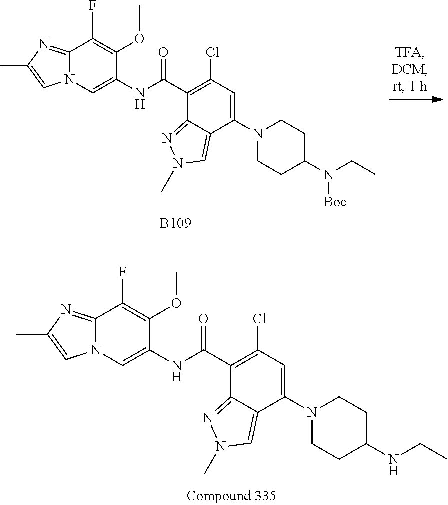

A solution of tert-butyl N-[1-[6-chloro-7-[(8-fluoro-7-methoxy-2-methyl-imidazo[1,2-a]-pyridin-6-yl)carbamoyl]-2-methyl-indazol-4-yl]-4-piperidyl]-N-ethyl-carbamate (90 mg, 146.6 μmol) and TFA (0.2 mL) in DCM (1 mL) was stirred for 1 h at room temperature. The mixture was neutralized to pH 7 with NH₃ in MeOH. The resulting mixture was concentrated under reduced pressure and purified by reversed-phase flash chromatography (Condition 3, Gradient 7) to afford 6-chloro-4-[4-(ethylamino)-1-piperidyl]-N-(8-fluoro-7-methoxy-2-methyl-imidazo[1,2-a]pyridin-6-yl)-2-methyl-indazole-7-carboxamide as a solid. LCMS (ES, m/z): 514 [M+H]⁺. ¹H NMR (400 MHz, DMSO-d₆) δ 11.49 (s, 1H), 9.45 (s, 1H), 8.81 (s, 1H), 7.80 (d, J=3.1 Hz, 1H), 6.37 (s, 1H), 4.25 (s, 3H), 4.17 (d, J=1.9 Hz, 3H), 3.88 (d, J=12.7 Hz, 2H), 3.09 (t, J=11.7 Hz, 2H), 2.69 (s, 1H), 2.61 (q, J=7.0 Hz, 2H), 2.32 (s, 3H), 1.95 (d, J=12.5 Hz, 2H), 1.42 (q, J=11.1 Hz, 2H), 1.04 (t, J=7.1 Hz, 3H).

### Example 125: Synthesis of C100

### Synthesis of C98

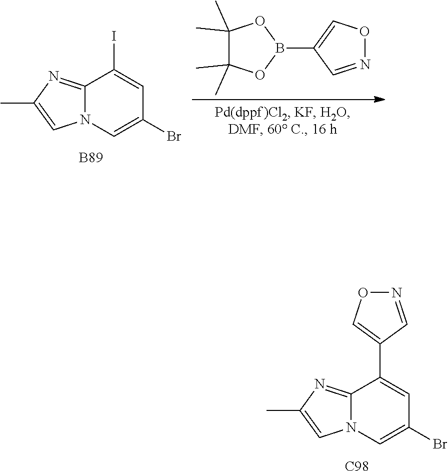 [SMILES: "B89" ; Cc1cn2cc(Br)cc(I)c2n1 ; CC1(C)OB(c2cnoc2)OC1(C)C ; Cc1cn2cc(Br)cc(-c3cnoc3)c2n1 ; "C98"]

To a stirred mixture of 6-bromo-8-iodo-2-methyl-imidazo[1,2-a]pyridine (2.3 g, 6.83 mmol) and 4-(4,4,5-trimethyl-1,3,2-dioxaborolan-2-yl) isoxazole (1.24 g, 6.83 mmol) in DMF (20 mL) and H₂O (2 mL) were added KF (1.19 g, 20.48 mmol) and Pd(dppf)Cl₂ (499 mg, 682.6 μmol), and the reaction was stirred for 16 h at 60° C. under N₂. The resulting mixture was allowed to cool to room temperature, diluted with water (100 mL), and extracted with EtOAc (2×100 mL). The combined organic layers were washed with brine (200 mL), dried over anhydrous Na₂SO₄, and filtered. The filtrate was concentrated under reduced pressure to afford 4-(6-bromo-2-methyl-imidazo[1,2-a]pyridin-8-yl) isoxazole (1.3 g, 4.67 mmol, 68% yield) as a solid. LCMS (ES, m/z): 278 [M+H]⁺.

### Synthesis of C99

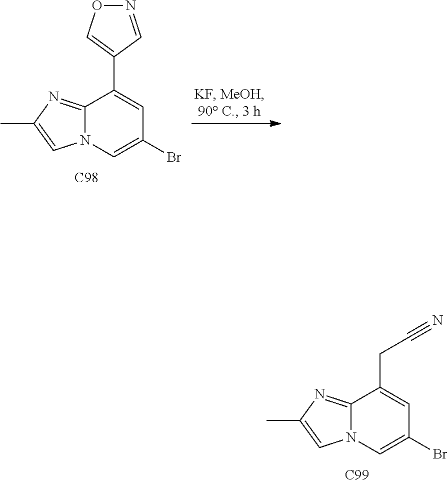 [SMILES: Cc1cn2cc(Br)cc(-c3cnoc3)c2n1 ; "C98" ; Cc1cn2cc(Br)cc(CC#N)c2n1 ; "C99"]

A solution of 4-(6-bromo-2-methyl-imidazo[1,2-a]pyridin-8-yl) isoxazole (1.25 g, 4.49 mmol) in MeOH (20 mL) was treated with KF (1.31 g, 22.47 mmol) at rt under N₂. The resulting mixture was stirred for 3 h at 90° C. under N₂. The resulting mixture was diluted with water (20 mL) and extracted with EtOAc (3×20 mL). The combined organic layers were washed with brine (40 mL), dried over anhydrous Na₂SO₄, and filtered. The filtrate was concentrated under reduced pressure, to afford 2-(6-bromo-2-methyl-imidazo[1,2-a]pyridin-8-yl) acetonitrile (530 mg, 2.12 mmol, 47% yield) as a solid. LCMS (ES, m/z): 250 [M+H]⁺.

### Synthesis of C100

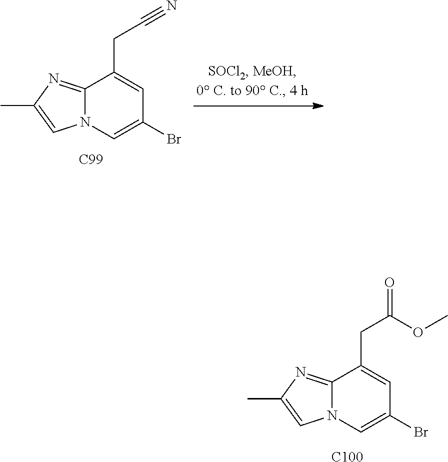

A solution of 2-(6-bromo-2-methyl-imidazo[1,2-a]pyridin-8-yl) acetonitrile (490 mg, 1.96 mmol) in MeOH (10 mL) was treated with SOCl₂ (233 mg, 1.96 mmol, 142.9 μL) at 0° C. under N₂. The resulting mixture was stirred for 4 h at 90° C. under N₂. The resulting mixture was concentrated under reduced pressure, diluted with water (20 mL), and extracted with EtOAc (3×20 mL). The combined organic layers were washed with brine (40 mL), dried over anhydrous Na₂SO₄, and filtered. The filtrate was concentrated under reduced pressure, to afford methyl 2-(6-bromo-2-methyl-imidazo[1,2-a]pyridin-8-yl)acetate (400 mg, 1.41 mmol, 72% yield) as a solid. LCMS (ES, m/z): 283 [M+H]⁺.

### Example 126: Synthesis of Compound 339

### Synthesis of B110

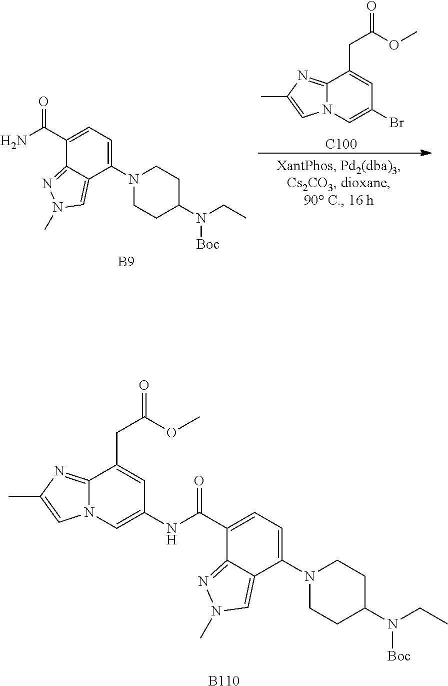

To a stirred mixture of methyl 2-(6-bromo-2-methyl-imidazo[1,2-a]pyridin-8-yl)acetate (528 mg, 1.87 mmol) and tert-butyl N-[1-(7-carbamoyl-2-methyl-indazol-4-yl)-4-piperidyl]-N-ethyl-carbamate (500 mg, 1.25 mmol) in dioxane (10 mL) were added Cs₂CO₃ (1.22 g, 3.74 mmol), XantPhos (144 mg, 249.1 μmol) and Pd₂(dba)₃ (114 mg, 124.5 μmol) at room temperature under N₂. The resulting mixture was stirred for 16 h at 90° C. under N₂. The resulting mixture was allowed to cool to room temperature, diluted with water (30 mL), and extracted with EtOAc (2×50 mL). The combined organic layers were washed with brine (50 mL), dried over anhydrous Na₂SO₄, and filtered. The filtrate was concentrated under reduced pressure and purified by silica gel column chromatography (100% EtOAc) to afford methyl 2-[6-[[4-[4-[tert-butoxycarbonyl(ethyl)amino]-1-piperidyl]-2-methyl-indazole-7-carbonyl]amino]-2-methyl-imidazo[1,2-a]pyridin-8-yl]acetate (240 mg, 397.5 μmol, 32% yield) as a solid. LCMS (ES, m/z): 604 [M+H]⁺.

### Synthesis of B111

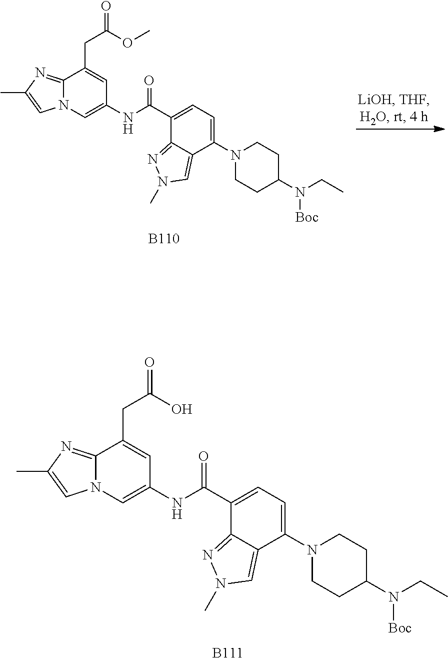

A solution of methyl 2-[6-[[4-[4-[tert-butoxycarbonyl(ethyl)amino]-1-piperidyl]-2-methyl-indazole-7-carbonyl]amino]-2-methyl-imidazo[1,2-a]pyridin-8-yl]acetate (80 mg, 132.5 μmol) in THF (0.5 mL) and H₂O (0.5 mL) was treated with LiOH (22 mg, 530.1 μmol), and the reaction was stirred for 4 h at room temperature. The resulting mixture was acidified to pH 3 with HCl (1N) and extracted with EtOAc (3×40 mL). The combined organic layers were washed with brine (50 mL), dried over anhydrous Na₂SO₄, and filtered. The filtrate was concentrated under reduced pressure to afford 2-[6-[[4-[4-[tert-butoxycarbonyl(ethyl)amino]-1-piperidyl]-2-methyl-indazole-7-carbonyl]amino]-2-methyl-imidazo[1,2-a]pyridin-8-yl]acetic acid (60 mg, 101.8 μmol, 77% yield) as a solid. LCMS (ES, m/z): 590 [M+H]⁺.

### Synthesis of Compound 339

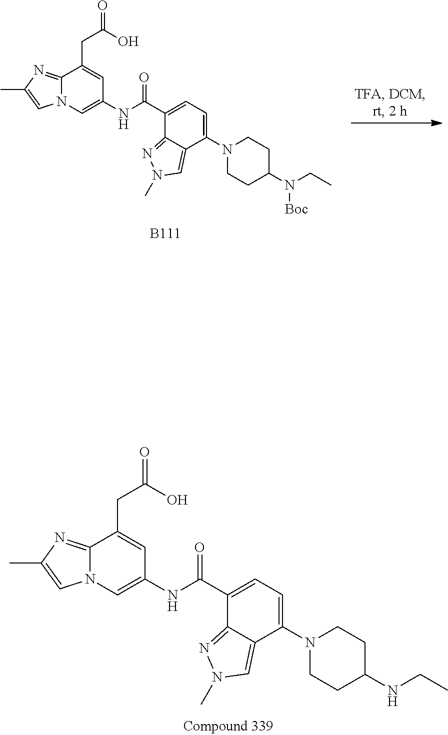

To a stirred mixture of 2-[6-[[4-[4-[tert-butoxycarbonyl(ethyl)amino]-1-piperidyl]-2-methyl-indazole-7-carbonyl]amino]-2-methyl-imidazo[1,2-a]pyridin-8-yl]acetic acid (60 mg, 101.8 μmol) in DCM (2 mL) was added TFA (0.5 mL) dropwise, and the reaction was stirred for 2 h at room temperature. The resulting mixture was concentrated under reduced pressure and purified by Prep-HPLC (Condition 5, Gradient 3) to afford 2-[6-[[4-[4-(ethylamino)-1-piperidyl]-2-methyl-indazole-7-carbonyl]amino]-2-methyl-imidazo[1,2-a]-pyridin-8-yl]acetic acid (Compound 339, 16 mg, 32.7 μmol, 32% yield) as a solid. LCMS (ES, m/z): 490 [M+H]⁺. 1H NMR (400 MHz, DMSO-d₆) δ 10.99 (s, 1H), 9.26 (s, 1H), 8.77 (s, 1H), 7.97 (d, J=8.1 Hz, 1H), 7.74 (s, 1H), 7.14 (s, 1H), 6.49 (d, J=8.2 Hz, 1H), 4.29 (s, 3H), 3.89 (d, J=13.0 Hz, 2H), 3.81 (s, 2H), 3.04 (t, J=11.9 Hz, 2H), 2.72 (s, 1H), 2.63 (t, J=7.2 Hz, 2H), 2.32 (s, 3H), 1.98 (d, J=12.9 Hz, 2H), 1.46 (d, J=12.1 Hz, 2H), 1.06 (t, J=7.1 Hz, 3H).

### Example 127: Synthesis of Compound 340

### Synthesis of B112

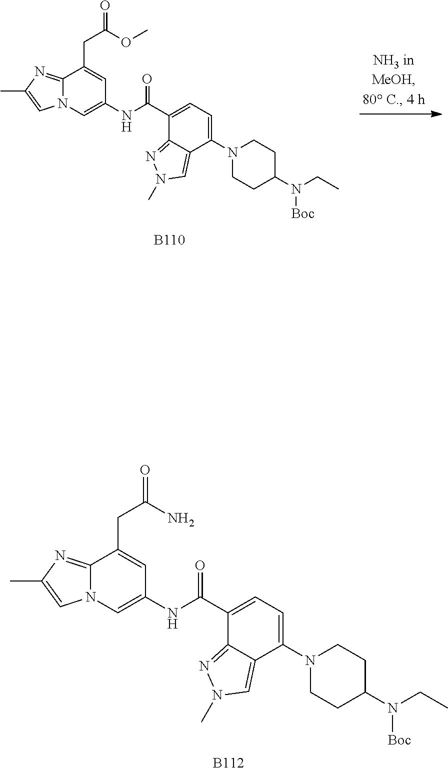

A solution of methyl 2-[6-[[4-[4-[tert-butoxycarbonyl(ethyl)amino]-1-piperidyl]-2-methyl-indazole-7-carbonyl]amino]-2-methyl-imidazo[1,2-a]pyridin-8-yl]acetate (70 mg, 116 μmol) in MeOH (1 mL) was treated with NH₃ (2.0 M in MeOH) (0.6 mL, 1.16 mmol) at rt. The resulting mixture was stirred for 4 h at 80° C. under N₂. The mixture was allowed to cool down to room temperature. The resulting mixture was concentrated under reduced pressure to afford tert-butyl N-[1-[7-[[8-(2-amino-2-oxo-ethyl)-2-methyl-imidazo[1,2-a]pyridin-6-yl]carbamoyl]-2-methyl-indazol-4-yl]-4-piperidyl]-N-ethyl-carbamate (65 mg, 110.4 μmol, 95% yield) as a solid. LCMS (ES, m/z): 589 [M+H]⁺.

### Synthesis of Compound 340

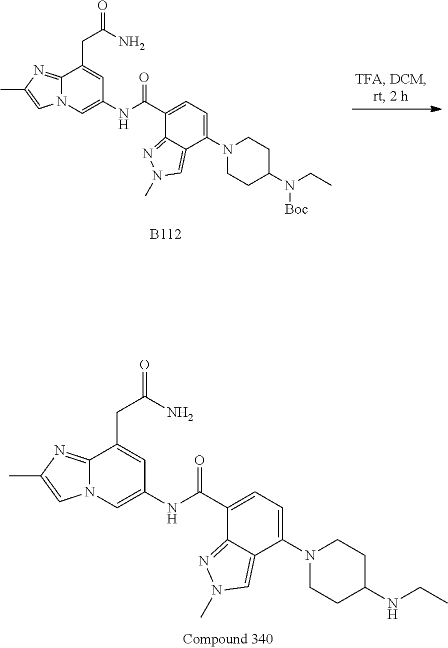

To a stirred mixture of tert-butyl N-[1-[7-[[8-(2-amino-2-oxo-ethyl)-2-methyl-imidazo[1,2-a]pyridin-6-yl]carbamoyl]-2-methyl-indazol-4-yl]-4-piperidyl]-N-ethyl-carbamate (60 mg, 101.9 μmol) in DCM (2 mL) was added TFA (0.5 mL) dropwise, and the reaction was stirred for 2 h at room temperature. The resulting mixture was concentrated under reduced pressure and purified by Prep-HPLC (Condition 5, Gradient 3) to afford N-[8-(2-amino-2-oxo-ethyl)-2-methyl-imidazo[1,2-a]pyridin-6-yl]-4-[4-(ethylamino)-1-piperidyl]-2-methyl-indazole-7-carboxamide (Compound 340, 12 mg, 24.6 μmol, 24% yield) as a solid. LCMS (ES, m/z): 489 [M+H]⁺. ¹H NMR (400 MHz, DMSO-d₆) δ 11.01 (s, 1H), 9.29 (d, J=1.9 Hz, 1H), 8.78 (s, 1H), 7.98 (d, J=8.1 Hz, 1H), 7.76 (d, J=1.0 Hz, 1H), 7.60 (s, 1H), 7.14 (d, J=1.9 Hz, 1H), 7.01 (s, 1H), 6.49 (d, J=8.2 Hz, 1H), 4.30 (s, 3H), 3.88 (d, J=12.8 Hz, 2H), 3.75 (s, 2H), 3.18-2.93 (m, 2H), 2.68 (q, J=5.1, 4.3 Hz, 1H), 2.61 (q, J=7.1 Hz, 2H), 2.33 (s, 3H), 1.96 (dd, J=13.3, 3.8 Hz, 2H), 1.54-1.33 (m, 2H), 1.04 (t, J=7.1 Hz, 3H).

An analogous method was followed to obtain the following compound.

| | Starting | |
|---|---|---|
| Compound | Material | Characterization |
| 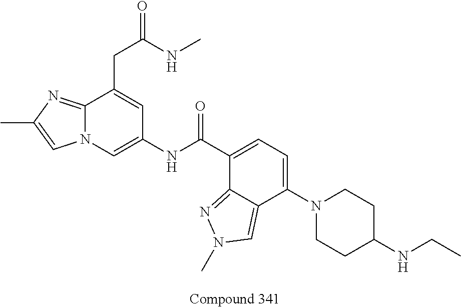 [SMILES: CCNC1CCN(c2ccc(C(=O)Nc3cc(CC(=O)NC)c4nc(C)cn4c3)c3nn(C)cc23)CC1] | MeNH₂ in MeOH | The crude product was purified by Prep- HPLC (Condition 2, Gradient 3) to afford 4- (4-(ethylamino)piperidin-1-yl)-2-methyl-N- (2-methyl-8-(2-(methylamino)-2- oxoethyl)imidazo[1,2-a]-pyridin-6-yl)-2H- indazole-7-carboxamide bis(2,2,2- trifluoroacetate) (Compound 341, 34 mg, 46.5 μmol, 47% yield) as a solid. LCMS (ES, m/z): 503 [M + H]⁺. ¹H NMR (400 MHz, DMSO-d₆) δ 14.20 (s, 1H), 11.34 (s, 1H), 9.70 (s, 1H), 8.89 (s, 1H), 8.54 (d, J = 7.2 Hz, 2H), 8.22 (s, 1H), 8.10 (d, J = 4.8 Hz, 1H), 8.03 (d, J = 8.1 Hz, 1H), 7.84 (s, 1H), 6.58 (d, J = 8.2 Hz, 1H), 4.32 (s, 3H), 4.07 (d, J = 13.0 Hz, 2H), 3.93 (s, 2H), 3.16- 2.97 (m, 4H), 2.65 (d, J = 4.5 Hz, 3H), 2.15 (d, J = 12.3 Hz, 2H), 1.80-1.58 (m, 2H), 1.24 (t, J = 7.2 Hz, 3H). |

### Example 128: Synthesis of C102

### Synthesis of C101

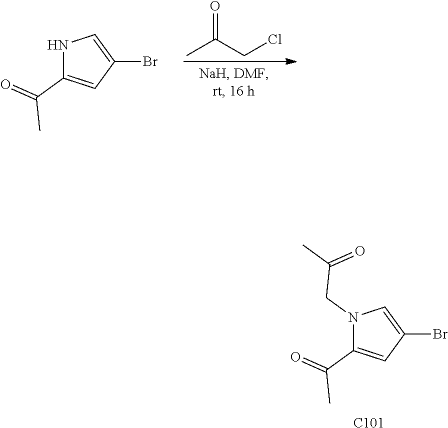

To a stirred solution of 1-(4-bromo-1H-pyrrol-2-yl) ethanone (1.9 g, 10.11 mmol) in DMF (20 mL) was added NaH (445 mg, 11.12 mmol, 60% purity) at 0° C. under N₂. The resulting mixture was stirred for 0.5 h at room temperature under N₂. To the above mixture was added 1-chloropropan-2-one (907 mg, 9.80 mmol) at 0° C. under N₂. The mixture was allowed to warm to room temperature and stirred for additional 16 h. The reaction was quenched by the addition of water (50 mL) at room temperature. The resulting mixture was extracted with EtOAc (3×30 mL). The combined organic layers were washed with brine (50 mL), dried over anhydrous Na₂SO₄, and filtered. The filtrate was concentrated under reduced pressure and purified by silica gel column chromatography (25% EtOAc in PE) to afford 1-(2-acetyl-4-bromo-pyrrol-1-yl)propan-2-one (480 mg, 1.97 mmol, 19% yield) as a solid. LCMS (ES, m/z): 244 [M+H].

### Synthesis of C102

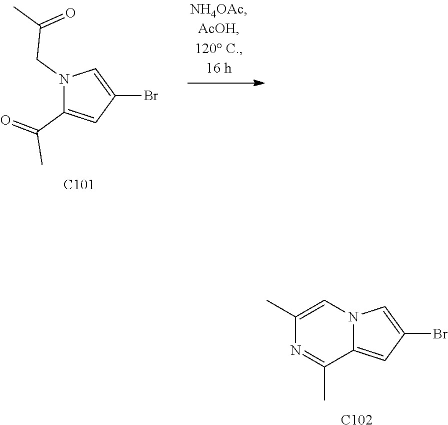

A solution of 1-(2-acetyl-4-bromo-pyrrol-1-yl)propan-2-one (486 mg, 1.99 mmol) and NH₄OAc (3.07 g, 39.82 mmol, 2.62 mL) in AcOH (20 mL) was stirred for 16 h at 120° C. The resulting mixture was concentrated under reduced pressure. The resulting mixture was diluted with EtOAc (50 mL). The mixture was basified to pH 8 with saturated aqueous NaHCO₃. The resulting mixture was extracted with EtOAc (3×20 mL). The combined organic layers were washed with brine (30 mL), dried over anhydrous Na₂SO₄, and filtered. The filtrate was concentrated under reduced pressure and purified by silica gel column chromatography (50% EtOAc in PE) to afford 7-bromo-1,3-dimethyl-pyrrolo[1,2-a]pyrazine (386 mg, 1.71 mmol, 86% yield) as a solid. LCMS (ES, m/z): 225 [M+H].

### Example 129: Synthesis of Compound 347

### Synthesis of B113

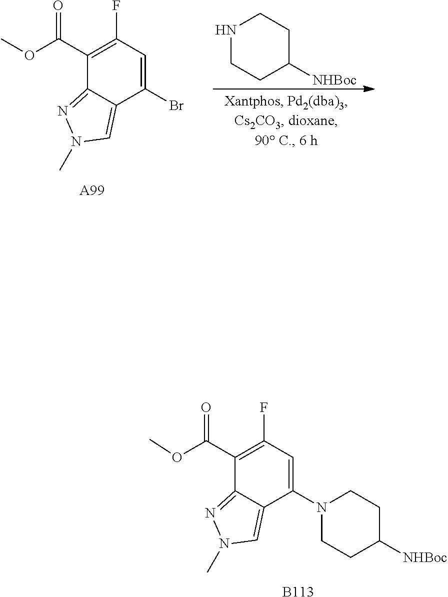

To a stirred mixture of methyl 4-bromo-6-fluoro-2-methyl-indazole-7-carboxylate (0.32 g, 1.11 mmol) and tert-butyl N-(4-piperidyl)carbamate (269 mg, 1.34 mmol) in Dioxane (5 mL) were added Cs₂CO₃ (726 mg, 2.23 mmol), Xantphos (130 mg, 222.9 μmol) and Pd₂(dba)₃ (65 mg, 111.5 μmol) at room temperature under N₂. The resulting mixture was stirred for 6 h at 90° C. under N₂. The mixture was allowed to cool down to room temperature, concentrated under reduced pressure and purified by silica gel column chromatography (86% EtOAc in PE) to afford methyl 4-[4-(tert-butoxycarbonylamino)-1-piperidyl]-6-fluoro-2-methyl-indazole-7-carboxylate (0.28 g, 688.9 μmol, 62% yield) as a solid. LCMS (ES, m/z): 407 [M+H]⁺.

### Synthesis of B114

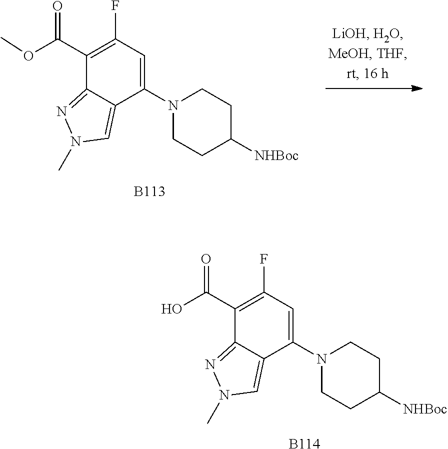 [SMILES: "B113" ; COC(=O)c1c(F)cc(N2CCC(NC(=O)OC(C)(C)C)CC2)c2cn(C)nc12 ; "B114" ; Cn1cc2c(N3CCC(NC(=O)OC(C)(C)C)CC3)cc(F)c(C(=O)O)c2n1]

To a stirred mixture of methyl 4-[4-(tert-butoxycarbonylamino)-1-piperidyl]-6-fluoro-2-methyl-indazole-7-carboxylate (0.26 g, 639.7 μmol) in MeOH (2 mL), THF (2 mL) and H₂O (1 mL) was added LiOH (76 mg, 3.20 mmol), and the reaction was stirred for 16 h at room temperature. The resulting mixture was neutralized to pH 5 with 1 N of HCl (aq.). The resulting mixture was diluted with water (10 mL) and extracted with EtOAc (2×20 mL). The combined organic layers were washed with water (2×40 mL), brine (40 mL), dried over anhydrous Na₂SO₄, and filtered. The filtrate was concentrated under reduced pressure, to afford 4-[4-(tert-butoxycarbonylamino)-1-piperidyl]-6-fluoro-2-methyl-indazole-7-carboxylic acid (0.23 g, 586.1 μmol, 92% yield) as a solid. LCMS (ES, m/z): 393 [M+H]⁺.

### Synthesis of B115

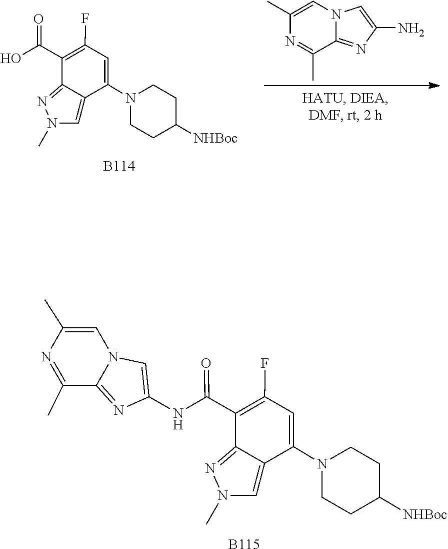

To a stirred mixture of 4-[4-(tert-butoxycarbonylamino)-1-piperidyl]-6-fluoro-2-methyl-indazole-7-carboxylic acid (0.11 g, 280.3 μmol) and 6,8-dimethylimidazo[1,2-a]pyrazin-2-amine (55 mg, 336.4 μmol) in DMF (2 mL) were added DIEA (108 mg, 840.9 μmol), HATU (160 mg, 420.5 μmol), and the reaction was stirred for 2 h at room temperature. The resulting mixture was diluted with water (10 mL) and extracted with EtOAc (2×20 mL). The combined organic layers were washed with water (2×40 mL), brine (40 mL), dried over anhydrous Na₂SO₄, and filtered. The filtrate was concentrated under reduced pressure and purified by silica gel column chromatography (75% EtOAc in PE) to afford tert-butyl N-[1-[7-[(6,8-dimethylimidazo[1,2-a]pyrazin-2-yl)carbamoyl]-6-fluoro-2-methyl-indazol-4-yl]-4-piperidyl]carbamate (0.06 g, 111.8 μmol, 40% yield) as a solid. LCMS (ES, m/z): 537 [M+H]⁺.

### Synthesis of Compound 347

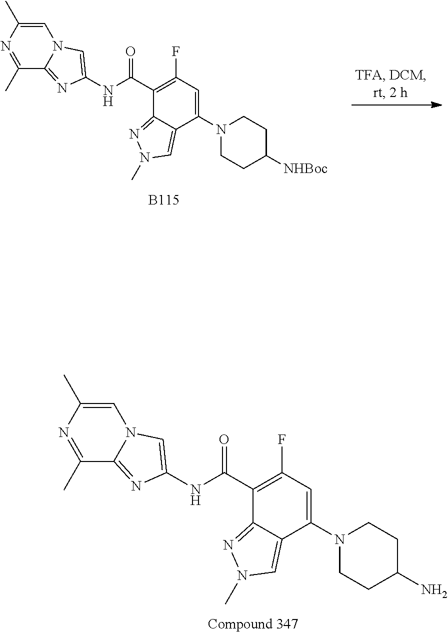

To a solution of tert-butyl N-[1-[7-[(6,8-dimethylimidazo[1,2-a]pyrazin-2-yl)carbamoyl]-6-fluoro-2-methyl-indazol-4-yl]-4-piperidyl]carbamate (0.06 g, 111.8 μmol) in DCM (1 mL) was added TFA (0.4 mL), and the reaction was stirred for 2 h at rt. The resulting mixture was concentrated under reduced pressure and adjusted to pH 8 with saturated NaHCO₃ (aq.). The resulting mixture was diluted with water (5 mL) and extracted with DCM/MeOH (20/1) (2×10 mL). The combined organic layers were washed with water (20 mL), brine (20 mL) and dried over anhydrous Na₂SO₄, and filtered. The filtrate was concentrated under reduced pressure and purified by Prep-HPLC (Condition 2, Gradient 4) to afford 4-(4-aminopiperidin-1-yl)-N-(6,8-dimethylimidazo[1,2-a]pyrazin-2-yl)-6-fluoro-2-methyl-2H-indazole-7-carboxamide 2,2,2-trifluoroacetate (0.03 g, 54.5 μmol, 49% yield) as a solid. LCMS (ES, m/z): 437 [M+H]′. ¹H NMR (300 MHz, DMSO-d₆) δ 11.63 (s, 1H), 8.87 (s, 1H), 8.39 (s, 2H), 7.95 (s, 3H), 6.33 (d, J=15.7 Hz, 1H), 4.25 (s, 3H), 4.05 (d, J=13.4 Hz, 2H), 3.35 (s, 1H), 3.13 (t, J=12.5 Hz, 2H), 2.74 (s, 3H), 2.40 (s, 3H), 2.03 (d, J=12.4 Hz, 2H), 1.71-1.63 (m, 1H).

### Example 130: Synthesis of C105

### Synthesis of C103

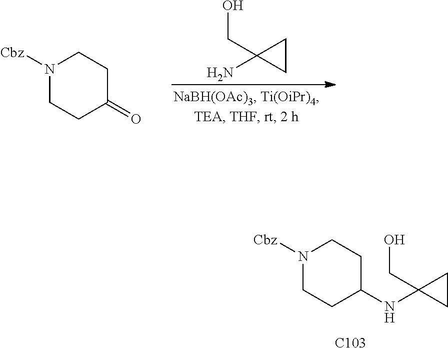 [SMILES: O=C1CCN(C(=O)OCc2ccccc2)CC1 ; NC1(CO)CC1 ; O=C(OCc1ccccc1)N1CCC(NC2(CO)CC2)CC1 ; "C103"]

A solution of benzyl 4-oxopiperidine-1-carboxylate (5 g, 21.44 mmol) and (1-aminocyclopropyl) methanol hydrochloride (3.18 g, 25.72 mmol) in THF (60 mL) was treated with Ti(OiPr)₄ (9.13 g, 32.15 mmol) and TEA (4.34 g, 42.87 mmol) for 30 min at rt under N₂ followed by the addition of NaBH(OAc)₃ (9.09 g, 42.87 mmol) in portions, and the reaction was stirred for an additional 2 h at rt. The reaction was quenched with water/ice at 0° C. and extracted with DCM (3×100 mL). The combined organic layers were washed with brine (2×100 mL), dried over anhydrous Na₂SO₄, and filtered. The filtrate was concentrated under reduced pressure and purified by silica gel column chromatography (9% MeOH in DCM) to afford benzyl 4-[[1-(hydroxymethyl)-cyclopropyl]amino]piperidine-1-carboxylate (4.5 g, 14.78 mmol, 69% yield) as a solid. LCMS (ES, m/z): 305 [M+H]⁺.

### Synthesis of C104

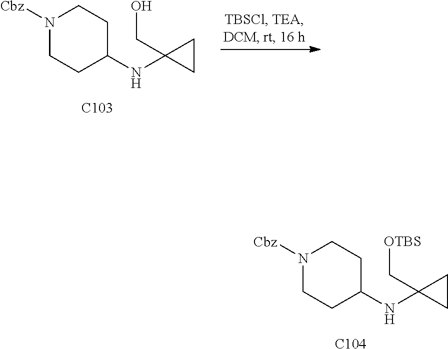 [SMILES: O=C(OCc1ccccc1)N1CCC(NC2(CO)CC2)CC1 ; "C103" ; CC(C)(C)[Si](C)(C)OCC1(NC2CCN(C(=O)OCc3ccccc3)CC2)CC1 ; "C104"]

A solution of benzyl 4-[[1-(hydroxymethyl)cyclopropyl]amino]piperidine-1-carboxylate (2 g, 6.57 mmol) and TEA (1.99 g, 19.71 mmol, 2.75 mL) in DCM (30 mL) was treated with TBSCl (1.19 g, 7.88 mmol) at 0° C. under N₂. The resulting mixture was stirred for additional 16 h at rt. The resulting mixture was concentrated under reduced pressure and purified by silica gel column chromatography (85% EtOAc in PE) to afford benzyl 4-[[1-[[tert-butyl(dimethyl)silyl]-oxymethyl]cyclopropyl]amino]piperidine-1-carboxylate (2.5 g, 5.97 mmol, 91% yield) as an oil. LCMS (ES, m/z): 419 [M+H]⁺.

### Synthesis of C105

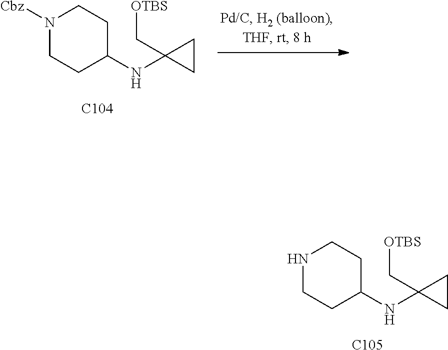 [SMILES: CC(C)(C)[Si](C)(C)OCC1(NC2CCN(C(=O)OCc3ccccc3)CC2)CC1 ; "C104" ; CC(C)(C)[Si](C)(C)OCC1(NC2CCNCC2)CC1 ; "C105"]

To a solution of benzyl 4-[[1-[[tert-butyl(dimethyl)silyl]oxymethyl]cyclopropyl]-amino]piperidine-1-carboxylate (0.6 g, 1.43 mmol) in 10 mL of THF was added Pd/C (10%, 228 mg) in 3-necked round-bottom flask. The mixture was hydrogenated at room temperature under 30 psi of hydrogen pressure for 8 h, filtered through a Celite pad and concentrated under reduced pressure to afford N-[1-[[tert-butyl(dimethyl)silyl]oxymethyl]cyclopropyl]piperidin-4-amine (0.32 g, 1.12 mmol, 79% yield) as an oil. LCMS (ES, m/z): 285 [M+H]⁺.

### Example 131: Synthesis of C107

### Synthesis of C106

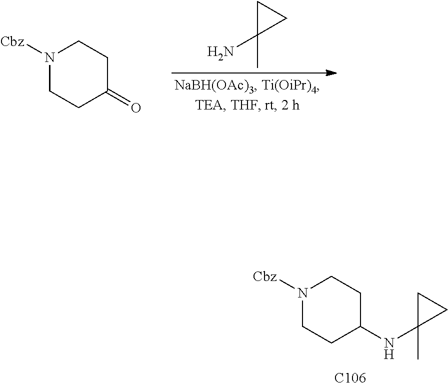

A solution of benzyl 4-oxopiperidine-1-carboxylate (1 g, 4.29 mmol, 853.2 μL) and 1-Methylcyclopropanamine hydrochloride (553 mg, 5.14 mmol) in THF (15 mL) was treated with Ti(OiPr)₄ (1.83 g, 6.43 mmol) and TEA (868 mg, 8.57 mmol) for 30 min at rt under N₂ followed by the addition of NaBH(OAc)₃ (1.82 g, 8.57 mmol) in portions at rt. The resulting mixture was stirred for additional 2 h at rt. The reaction was quenched with water/ice at 0° C. The resulting mixture was extracted with DCM (3×50 mL). The combined organic layers were washed with brine (2×50 mL), dried over anhydrous Na₂SO₄, and filtered. The filtrate was concentrated under reduced pressure and purified by silica gel column chromatography (9% MeOH in DCM) to afford benzyl 4-[(1-methylcyclopropyl)-amino]piperidine-1-carboxylate (0.8 g, 2.77 mmol, 65% yield) as a solid. LCMS (ES, m/z): 289 [M+H]⁺.

### Synthesis of C107

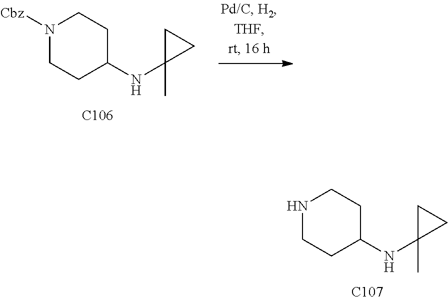 [SMILES: CC1(NC2CCN(C(=O)OCc3ccccc3)CC2)CC1 ; "C106" ; CC1(NC2CCNCC2)CC1 ; "C107"]

To a solution of benzyl 4-[(1-methylcyclopropyl)amino]piperidine-1-carboxylate (400 mg, 1.39 mmol) in 10 mL of THF was added Pd/C (40 mg, 10%). The mixture was hydrogenated at room temperature under 30 psi of hydrogen pressure for 16 h, filtered through a Celite pad and concentrated under reduced pressure to afford N-(1-methylcyclopropyl)piperidin-4-amine (0.22 g, 1.43 mmol, 103% yield) as an oil. LCMS (ES, m/z): 155 [M+H]⁺.

### Example 132: Synthesis of Compound 356

### Synthesis of B116

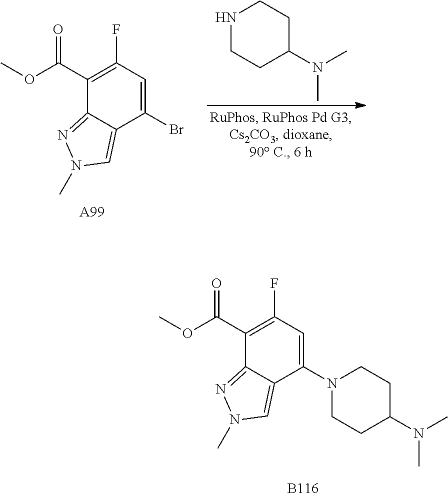

To a stirred mixture of methyl 4-bromo-6-fluoro-2-methyl-indazole-7-carboxylate (300 mg, 1.04 mmol) in dioxane (4 mL) were added N,N-dimethylpiperidin-4-amine (147 mg, 1.15 mmol), Cs₂CO₃ (681 mg, 2.09 mmol), Ruphos (98 mg, 209.0 μmol) and RuPhos Pd G3 (87 mg, 104.5 μmol), and the reaction was stirred for 6 h at 90° C. under N₂. The resulting mixture was diluted with water (15 mL) and extracted with DCM (2×15 mL). The combined organic layers were washed with water (2×20 mL), dried over anhydrous Na₂SO₄, and filtered. The filtrate was concentrated under reduced pressure and purified by silica gel column chromatography (5% MeOH in DCM) to afford methyl 4-[4-(dimethylamino)-1-piperidyl]-6-fluoro-2-methylindazole-7-carboxylate (B116, 300 mg, 897.2 μmol, 86% yield) as an oil. LCMS (ES, m/z): 335 [M+H]⁺.

### Synthesis of B117

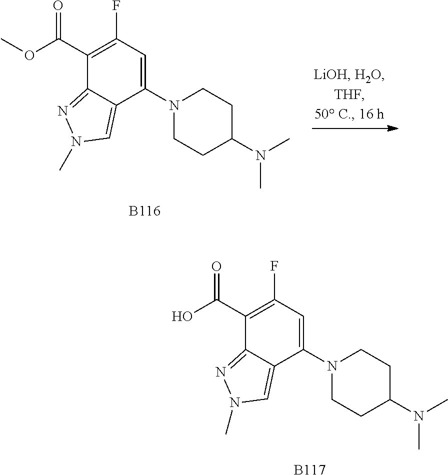

To a solution of methyl 4-[4-(dimethylamino)-1-piperidyl]-6-fluoro-2-methyl-indazole-7-carboxylate (280 mg, 837.4 μmol) in THF (2 mL), H₂O (1 mL) was added LiOH·H₂O (176 mg, 4.19 mmol), and the mixture was stirred for 16 h at 50° C. The resulting mixture was diluted with water and adjusted to pH 5-6 with 1M HCl. The resulting mixture was extracted with DCM (2×25 mL). The combined organic layers were washed with water (2×50 mL), dried over anhydrous Na₂SO₄, and filtered. The filtrate was concentrated under reduced pressure to afford 4-[4-(dimethylamino)-1-piperidyl]-6-fluoro-2-methyl-indazole-7-carboxylic acid (180 mg, 561.9 μmol, 67% yield) as a solid. LCMS (ES, m/z): 321 [M+H]⁺.

### Synthesis of Compound 356

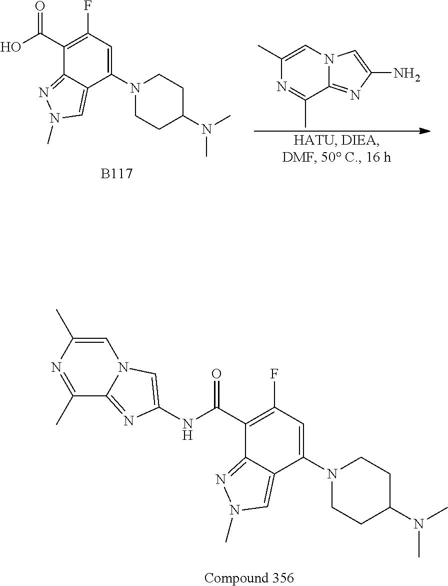 [SMILES: "B117" ; CN(C)C1CCN(c2cc(F)c(C(=O)O)c3nn(C)cc23)CC1 ; Cc1cn2cc(NC(=O)c3c(F)cc(N4CCC(N(C)C)CC4)c4cn(C)nc34)nc2c(C)n1 ; Cc1cn2cc(N)nc2c(C)n1 ; "Compound 356"]

To a solution of 4-[4-(dimethylamino)-1-piperidyl]-6-fluoro-2-methyl-indazole-7-carboxylic acid (90 mg, 280.9 μmol) in DMF (1 mL) were added 6,8-dimethylimidazo[1,2-a]-pyrazin-2-amine (55 mg, 337.1 μmol), DIEA (145 mg, 1.12 mmol) and HATU (160 mg, 421.4 μmol), and the mixture was stirred for 16 h at 50° C. The reaction was diluted with water (15 mL), and the resulting mixture was extracted with EtOAc (2×15 mL). The combined organic layers were washed with water (3×20 mL), dried over anhydrous Na₂SO₄, and filtered. The filtrate was concentrated under reduced pressure and purified by Prep-HPLC (Condition 5, Gradient 18) to afford 4-[4-(dimethylamino)-1-piperidyl]-N-(6,8-dimethylimidazo[1,2-a]pyrazin-2-yl)-6-fluoro-2-methyl-indazole-7-carboxamide (Compound 356, 35 mg, 75.3 μmol, 27% yield) as a solid. LCMS (ES, m/z): 465 [M+H]⁺. ¹H NMR (400 MHz, DMSO-d₆) δ 11.56 (s, 1H), 8.83 (s, 1H), 8.30 (d, J=9.0 Hz, 2H), 6.25 (d, J=15.9 Hz, 1H), 4.24 (s, 3H), 4.00 (d, J=12.8 Hz, 2H), 3.10-2.99 (m, 2H), 2.69 (s, 3H), 2.43-2.31 (m, 4H), 2.22 (s, 6H), 1.90 (d, J=12.9 Hz, 2H), 1.63-1.46 (m, 2H).

### Example 133: Synthesis of C108

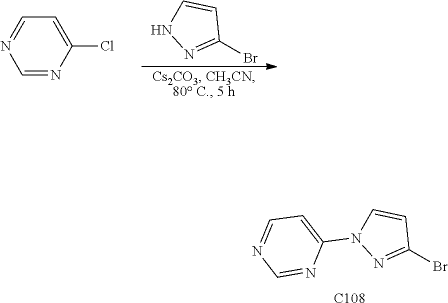

To a stirred mixture of 4-chloropyrimidine (1.0 g, 8.73 mmol) and Cs₂CO₃ (8.53 g, 26.19 mmol) in MeCN (20 mL) was added 3-bromo-1H-pyrazole (1.67 g, 11.35 mmol), and the reaction was stirred for 5 h at 80° C. The resulting mixture was diluted with water (200 mL) and extracted with EtOAc (2×200 mL). The combined organic layers were washed with brine (200 mL), dried over anhydrous Na₂SO₄, and filtered. The filtrate was concentrated under reduced pressure and purified by silica gel column chromatography (17% EtOAc in PE) to afford 4-(3-bromopyrazol-1-yl)pyrimidine (C108, 800 mg, 3.6 mmol, 41% yield) as a solid. LCMS (ES, m/z): 225 [M+H]⁺.

### Example 134: Synthesis of C111

### Synthesis of C109

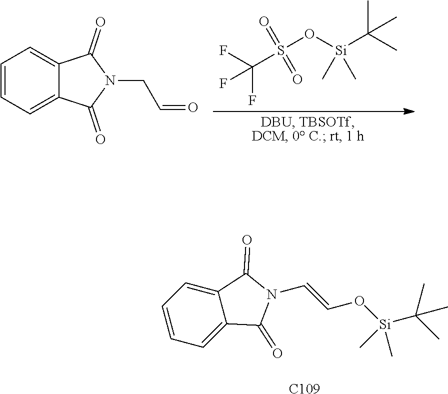

To a solution of 2-(1,3-dioxoisoindolin-2-yl) acetaldehyde (25 g, 82.48 mmol) in DCM (500 mL) were added DBU (37.61 g, 247.4 mmol) and TBSOTf (43.55 g, 165 mmol) dropwise at 0° C. The reaction mixture was warmed to 25° C. and stirred for 1 h. The reaction mixture was washed with water (2×500 mL). The organic layer was dried over anhydrous Na₂SO₄, and filtered. The filtrate was concentrated under reduced pressure and purified by silica gel column chromatography (9% EtOAc in PE) to afford (E)-2-(2-((tert-butyl dimethylsilyl)oxy)-vinyl) isoindoline-1,3-dione (3.5 g, 11.55 mmol, 9% yield) as an oil. ¹H NMR (400 MHz, CDCl₃) δ 7.85 (dd, J=5.4, 3.1 Hz, 2H), 7.72 (dd, J=5.4, 3.0 Hz, 2H), 7.56 (d, J=11.4 Hz, 1H), 6.44 (d, J=11.4 Hz, 1H), 0.99 (s, 9H), 0.23 (s, 4H).

### Synthesis of C110

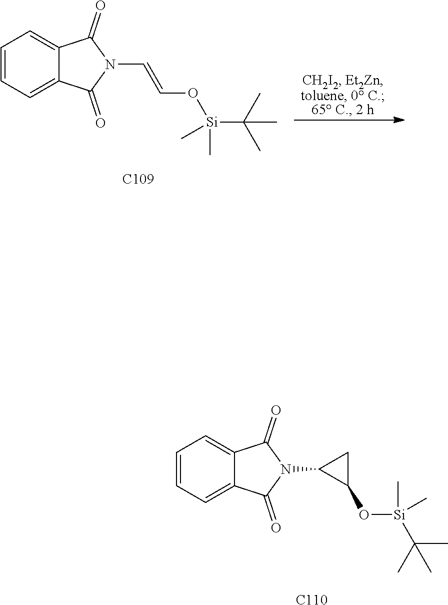 [SMILES: CC(C)(C)[Si](C)(C)O/C=C/N1C(=O)c2ccccc2C1=O ; "CH2I2, Et2Zn,toluene, 0° C.;65° C., 2 h" ; "C109" ; CC(C)(C)[Si](C)(C)O[C@@H]1C[C@H]1N1C(=O)c2ccccc2C1=O ; "C110"]

To a solution of (E)-2-(2-((tert-butyldimethylsilyl)oxy) vinyl) isoindoline-1,3-dione (3.5 g, 11.55 mmol) in toluene (100 mL) were added diethyl zinc (21.13 g, 173.3 mmol) and CH₂I₂ (46.39 g, 173.3 mmol) dropwise over 30 min at 0° C. under N₂. The reaction mixture was stirred at 65° C. for 2 h. The resulting mixture was transferred into 2 N sulphuric acid solution and product was extracted with EtOAc (2×100 mL). The combined organic layer was washed with saturated sodium bicarbonate (200 mL), water (200 mL) and brine (200 mL). The organic layer was dried over anhydrous Na₂SO₄, and filtered. The filtrate was concentrated under reduced pressure and purified by silica gel column chromatography (5% EtOAc in PE) to afford trans-2-(2-((tert-butyldimethylsilyl)oxy)cyclopropyl) isoindoline-1,3-dione (1.0 g, 3.15 mmol, 27% yield) as a solid and cis-2-(2-((tert-butyldimethylsilyl)-oxy)cyclopropyl) isoindoline-1,3-dione (300 mg, 0.95 mmol, 8% yield) as a solid.

### NMR for Trans-Target Compound:

¹H NMR (300 MHz, DMSO-d₆) δ 7.85-7.79 (m, 4H), 3.82 (ddd, J=6.7, 4.6, 1.7 Hz, 1H), 2.66 (ddd, J=7.3, 5.4, 1.7 Hz, 1H), 1.22-1.05 (m, 2H), 0.90 (s, 9H), 0.19 (d, J=2.1 Hz, 6H).

### NMR for Cis-Target Compound:

¹H NMR (300 MHz, DMSO-d₆) δ 7.86-7.81 (m, 4H), 3.68 (ddd, J=6.1, 5.2, 3.9 Hz, 1H), 2.67 (dt, J=8.9, 5.2 Hz, 1H), 1.37 (ddd, J=7.4, 5.2, 3.9 Hz, 1H), 1.22 (ddd, J=8.9, 7.5, 6.2 Hz, 1H), 0.62 (s, 9H), 0.00 (d, J=6.2 Hz, 6H).

### Synthesis of C111

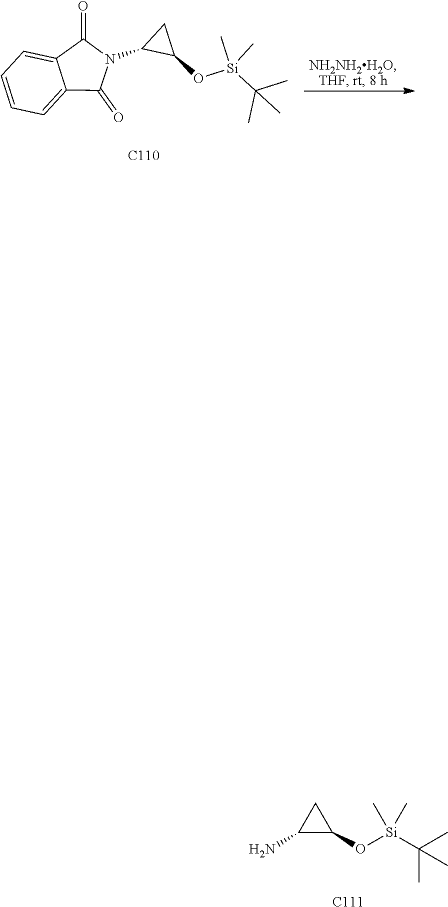 [SMILES: CC(C)(C)[Si](C)(C)O[C@@H]1C[C@H]1N1C(=O)c2ccccc2C1=O ; "NH2NH2·H2O,THF, rt, 8 h" ; "C110" ; CC(C)(C)[Si](C)(C)O[C@@H]1C[C@H]1N ; "C111"]

To a solution of 2-[(1R,2R)-2-[tert-butyl(dimethyl)silyl]oxycyclopropyl]isoindoline-1,3-dione (0.3 g, 945 μmol) in THF (3 mL) was added NH₂NH₂·H₂O (80%) (1 mL), and the reaction was stirred for 8 h at rt. The resulting mixture was diluted with water (8 mL) and extracted with EtOAc (2×10 mL). The combined organic layers were washed with water (2×20 mL), brine (30 mL), dried over anhydrous Na₂SO₄, and filtered. The filtrate was concentrated under reduced pressure, to afford (1R,2R)-2-[tert-butyl(dimethyl)silyl]-oxycyclopropanamine (0.15 g, 773.9 μmol, 82% yield) as an oil. ¹H NMR (300 MHz, DMSO-d₆) δ 3.08 (ddd, J=6.8, 3.4, 1.5 Hz, 1H), 2.16 (ddd, J=8.2, 4.4, 1.4 Hz, 1H), 1.76 (s, 2H), 0.86 (s, 9H), 0.53 (ddd, J=8.2, 5.6, 3.3 Hz, 1H), 0.41 (ddd, J=6.8, 5.6, 4.4 Hz, 1H), 0.07 (d, J=2.2 Hz, 6H).

An analogous method was followed to obtain the following compound.

| Compound | Characterization |
|---|---|
| 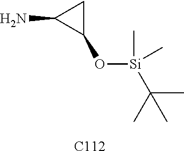 | The filtrate was concentrated under reduced pressure to afford (1S,2R)-2- [tert-butyl(dimethyl)silyl]-oxycyclopropanamine (0.15 g, 800.6 μmol, 85% yield) as an oil. ¹H NMR (300 MHz, DMSO-d₆) δ 3.27 (td, J = 6.4, 3.5 Hz, 1H), 1.97 (ddd, J = 7.8, 6.1, 4.9 Hz, 1H), 1.45 (s, 2H), 0.89 (s, 9H), 0.64 (dt, J = 7.8, 6.3 Hz, 1H), 0.11 (d, J = 2.5 Hz, 5H), −0.01 (ddd, J = 8.6, 6.5, 3.6 Hz, 1H). |

### Example 135: Synthesis of Compound 370

### Synthesis of B118

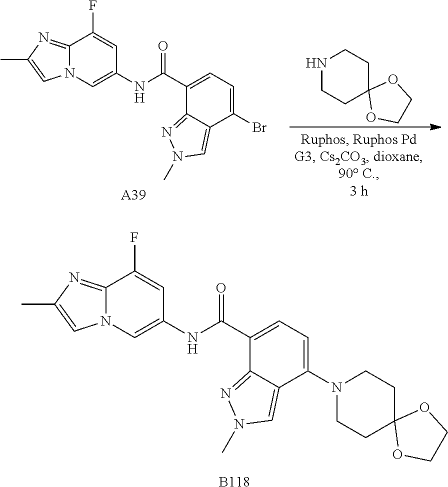

To a stirred mixture of 4-bromo-N-(8-fluoro-2-methyl-imidazo[1,2-a]pyridin-6-yl)-2-methyl-indazole-7-carboxamide (1.5 g, 3.73 mmol) and 1,4-dioxa-8-azaspiro[4.5]decane (641 mg, 4.48 mmol, 573.7 μL) in Dioxane (15 mL) were added Cs₂CO₃ (2.43 g, 7.46 mmol), Ruphos (348 mg, 745.9 μmol) and RuPhos Pd G3 (312 mg, 372.9 μmol) at room temperature under N₂. The resulting mixture was stirred for 3 h at 90° C. under N₂. The mixture was concentrated under reduced pressure and purified by silica gel column chromatography (89% EtOAc in PE) to afford 4-(1,4-dioxa-8-azaspiro[4.5]decan-8-yl)-N-(8-fluoro-2-methyl-imidazo[1,2-a]pyridin-6-yl)-2-methyl-indazole-7-carboxamide (1.25 g, 2.69 mmol, 72% yield) as a solid. LCMS (ES, m/z): 465 [M+H]⁺.

### Synthesis of B119

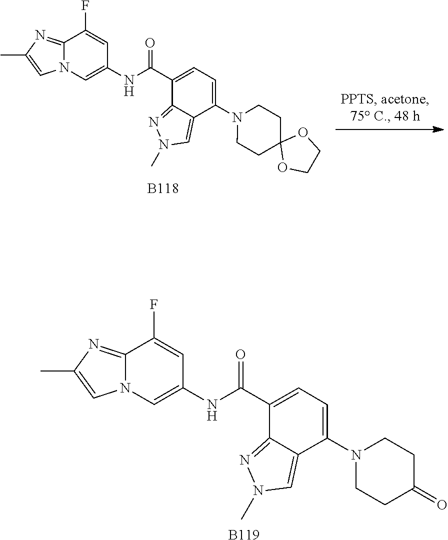 [SMILES: "B118" ; "B119" ; Cc1cn2cc(NC(=O)c3ccc(N4CCC5(CC4)OCCO5)c4cn(C)nc34)cc(F)c2n1 ; Cc1cn2cc(NC(=O)c3ccc(N4CCC(=O)CC4)c4cn(C)nc34)cc(F)c2n1]

To a solution of 4-(1,4-dioxa-8-azaspiro[4.5]decan-8-yl)-N-(8-fluoro-2-methyl-imidazo[1,2-a]pyridin-6-yl)-2-methyl-indazole-7-carboxamide (1.2 g, 2.58 mmol) in acetone (6 mL) was added PPTS (6.49 g, 25.83 mmol), and the reaction was stirred for 48 h at 75° C. The resulting mixture was concentrated under reduced pressure and purified by silica gel column chromatography (83% EtOAc in PE) to afford N-(8-fluoro-2-methyl-imidazo[1,2-a]pyridin-6-yl)-2-methyl-4-(4-oxo-1-piperidyl)indazole-7-carboxamide (1 g, 2.38 mmol, 92% yield) as a solid. LCMS (ES, m/z): 421 [M+H]⁺.

### Synthesis of B120

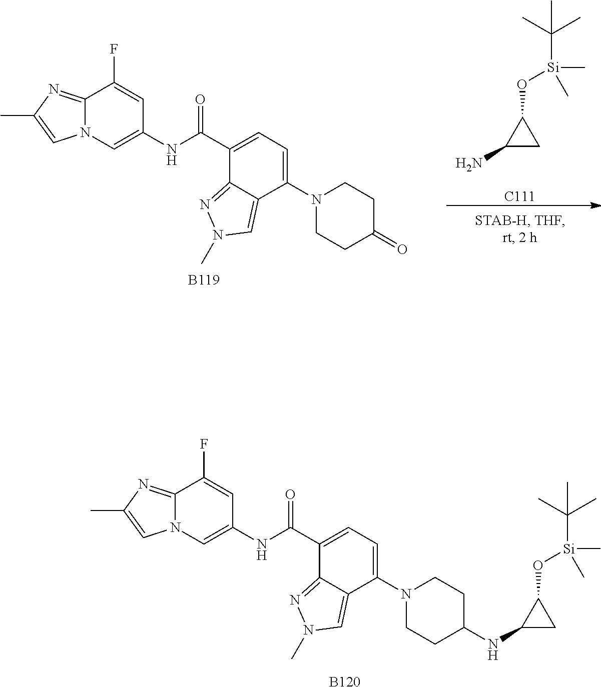

To a stirred mixture of N-(8-fluoro-2-methyl-imidazo[1,2-a]pyridin-6-yl)-2-methyl-4-(4-oxo-1-piperidyl)indazole-7-carboxamide (0.2 g, 475.7 μmol) and (1R,2R)-2-[tert-butyl(dimethyl)silyl]oxycyclopropanamine (107 mg, 570.8 μmol) in THF (5 mL) were added NaBH(OAc)₃ (202 mg, 951.4 μmol), and the reaction was stirred for 2 h at room temperature. The resulting mixture was diluted with water (10 mL) and extracted with EtOAc (2×20 mL). The combined organic layers were washed with water (2×40 mL), brine (40 mL), dried over anhydrous Na₂SO₄, and filtered. The filtrate was concentrated under reduced pressure, to afford 4-[4-[[(1R,2R)-2-[tert-butyl(dimethyl)silyl]oxycyclopropyl]amino]-1-piperidyl]-N—[(E)-2-[2-(fluoromethyl)-4-methyl-imidazol-1-yl]vinyl]-2-methyl-indazole-7-carboxamide (0.18 g, 309.4 μmol, 65% yield) as a solid. LCMS (ES, m/z): 592 [M+H]⁺.

### Synthesis of Compound 370

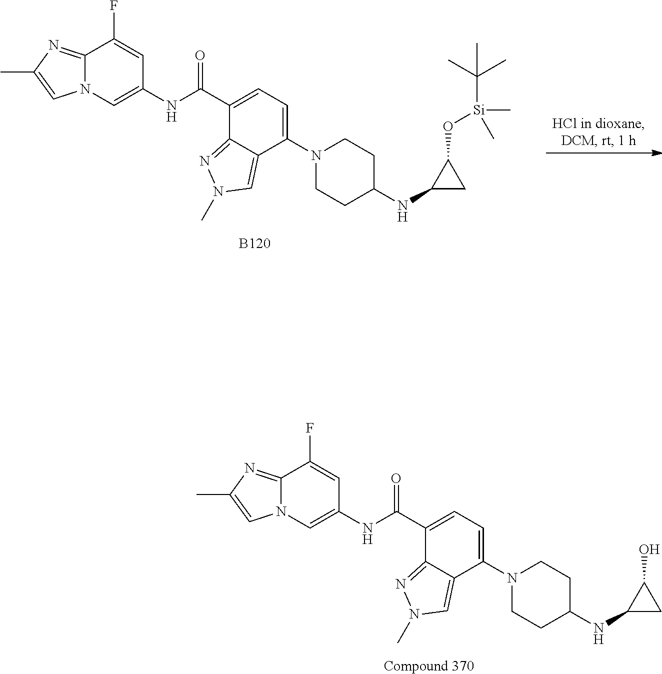

To a solution of 4-[4-[[(1R,2R)-2-[tert-butyl(dimethyl)silyl]oxycyclopropyl]amino]-1-piperidyl]-N-(8-fluoro-2-methyl-imidazo[1,2-a]pyridin-6-yl)-2-methyl-indazole-7-carboxamide (0.05 g, 84.49 μmol) in DCM (2 mL) was added HCl (4 M in dioxane) (1 mL). The reaction was stirred for 1 h at rt. The resulting mixture was concentrated under reduced pressure and purified by trituration with MTBE (3 mL). The resulting solid was filtered and dried to afford N-(8-fluoro-2-methylimidazo[1,2-a]pyridin-6-yl)-4-(4-(((1R,2R)-2-hydroxycyclopropyl)amino)piperidin-1-yl)-2-methyl-2H-indazole-7-carboxamide hydrochloride (Compound 370, 0.015 g, 29.2 μmol, 35% yield) as a solid. LCMS (ES, m/z): 478 [M+H]⁺. ¹H NMR (400 MHz, DMSO-d₆) δ 11.35 (s, 1H), 9.60 (d, J=1.5 Hz, 1H), 9.58-9.46 (m, 2H), 8.88 (s, 1H), 8.27 (dd, J=2.4, 1.2 Hz, 1H), 8.12 (dd, J=11.8, 1.6 Hz, 1H), 7.99 (d, J=8.1 Hz, 1H), 6.54 (d, J=8.3 Hz, 1H), 4.30 (s, 3H), 4.10-4.02 (m, 2H), 3.72 (ddd, J=7.6, 4.1, 1.7 Hz, 2H), 3.41 (s, 1H), 3.03 (t, J=12.8 Hz, 2H), 2.64 (s, 1H), 2.50 (s, 3H), 2.25 (dd, J=25.6, 12.7 Hz, 3H), 1.81 (dd, J=14.1, 10.5 Hz, 2H), 1.11 (td, J=7.0, 4.5 Hz, 1H), 1.01-0.89 (m, 1H).

An analogous method was followed to obtain the following compounds.

| | Starting | |
|---|---|---|
| Compound | Material | Characterization |
| 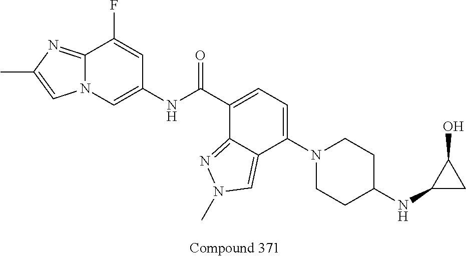 [SMILES: Cc1cn2cc(NC(=O)c3ccc(N4CCC(N[C@@H]5C[C@@H]5O)CC4)c4cn(C)nc34)cc(F)c2n1] | 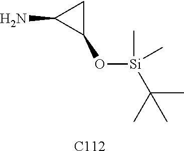 | The resulting mixture was concentrated under reduced pressure and purified by trituration with MTBE (3 mL). The resulting solid was filtered and dried to N-(8-fluoro-2-methyl- imidazo-[1,2-a]pyridin-6-yl)-4- (4-(((1R,2S)-2-hydroxy- cyclopropyl)- amino)piperidin-1-yl)- 2-methyl-2H- indazole-7-carboxamide hydrochloride (Compound 371, 0.006 g, 11.7 μmol, 9% yield) as a solid. LCMS (ES, m/z): 478 [M + H]⁺. |
| | | ¹H NMR (400 MHz, |
| | | Methanol-d₄) δ |
| | | 9.57 (d, J = 1.5 Hz, |
| | | 1H), 8.65 (s, 1H), |
| | | 8.21-8.07 (m, 3H), |
| | | 6.65 (d, J = 8.2 Hz, |
| | | 1H), 4.38 (s, 3H), 4.18 |
| | | (d, J = 13.2 Hz, |
| | | 2H), 3.81 (s, 1H), |
| | | 3.70 (ddd, J = 6.4, |
| | | 5.5, 3.7 Hz, 1H), |
| | | 3.17 (t, J = 12.7 Hz, |
| | | 2H), 2.73 (dt, J = |
| | | 8.4, 5.2 Hz, 1H), |
| | | 2.60 (d, J = 1.0 Hz, |
| | | 3H), 2.42-2.35 (m, |
| | | 2H), 2.05-1.91 (m, |
| | | 2H), 1.17 (td, J = |
| | | 7.9, 6.4 Hz, 1H), |
| | | 0.95-0.87 (m, 1H). |

### Example 136: Synthesis of Compound 376

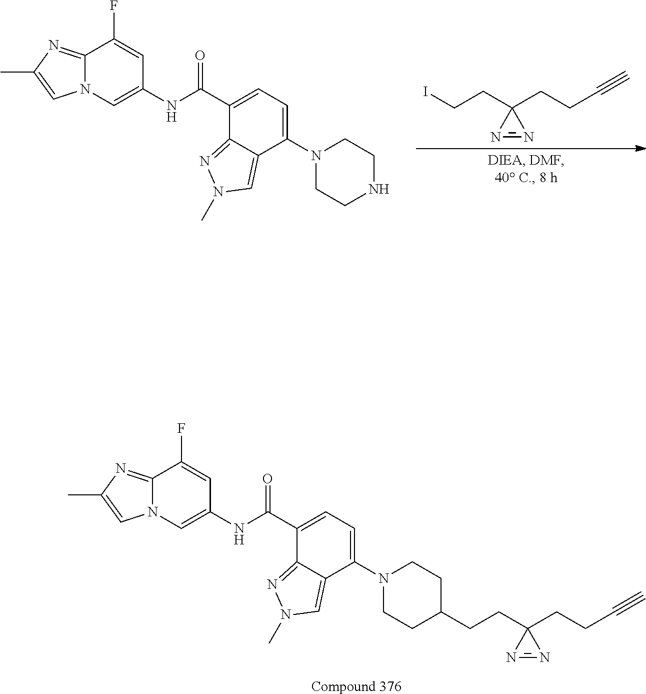

A mixture of N-(8-fluoro-2-methyl-imidazo[1,2-a]pyridin-6-yl)-2-methyl-4-piperazin-1-yl-indazole-7-carboxamide (95 mg, 233.2 μmol), 3-but-3-ynyl-3-(2-iodoethyl)diazirine (70 mg, 279.8 μmol) and DIEA (91 mg, 699.5 μmol) in DMF (2 mL) was stirred for 8 h at 40° C. The resulting mixture was purified by reversed-phase flash chromatography (Condition 4, Gradient 4) to afford 4-(4-(2-(3-(but-3-yn-1-yl)-3H-diazirin-3-yl)ethyl) piperazin-1-yl)-N-(8-fluoro-2-methyl-imidazo-[1,2-a]pyridin-6-yl)-2-methyl-2H-indazole-7-carboxamide bis(2,2,2-trifluoroacetate) (Compound 376, 39.4 mg, 52.1 μmol, 22% yield) as a solid. LCMS (ES, m/z): 528 [M+H-TFA-TFA]⁺. ¹H NMR (400 MHz, DMSO-d₆) δ 11.17 (s, 1H), 9.86 (s, 1H), 9.36 (s, 1H), 8.92 (s, 1H), 8.07-8.00 (m, 2H), 7.64 (d, J=12.5 Hz, 1H), 6.63 (d, J=8.1 Hz, 1H), 4.33 (s, 3H), 4.05 (s, 2H), 3.60 (s, 2H), 3.24 (s, 5H), 2.92 (t, J=2.6 Hz, 1H), 2.41 (s, 3H), 2.07 (td, J=7.4, 2.7 Hz, 2H), 1.89-1.82 (m, 2H), 1.68 (t, J=7.3 Hz, 2H).

An analogous method was followed to obtain the following compounds.

| Compound | Starting Material | Characterization |
|---|---|---|
| 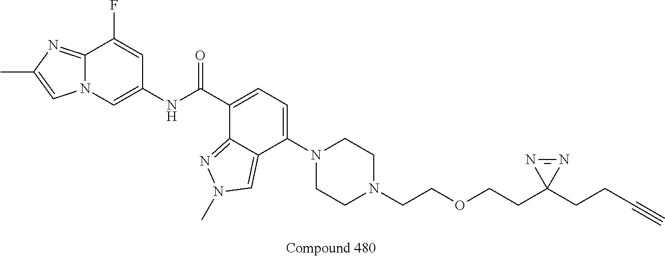 [SMILES: C#CCCC1(CCOCCN2CCN(c3ccc(C(=O)Nc4cc(F)c5nc(C)cn5c4)c4nn(C)cc34)CC2)N=N1] | 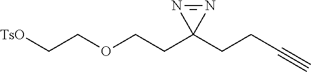 [SMILES: C#CCCC1(CCOCCOS(=O)(=O)c2ccc(C)cc2)N=N1] | Modification: The reaction was ran for 48 h. The residue was purified by reversed- phase flash chromatography (Condition 3, Gradient 6) to afford 4-[4-[2-[2- (3-but-3-ynyldiazirin- 3-yl)ethoxy]ethyl]- piperazin-1-yl]-N-(8- fluoro-2-methyl- imidazo[1,2-a]-pyridin- 6-yl)-2-methyl- indazole-7- |
| | | carboxamide |
| | | (Compound 480, 40 |
| | | mg, 70 μmol). LCMS |
| | | (ES, m/z): 572 [M + H]⁺. |
| | | ¹H NMR (400 MHz, |
| | | DMSO-d₆) δ 11.08 (s, |
| | | 1H), 9.22 (d, J = 1.6 Hz, |
| | | 1H), 8.81 (s, 1H), 7.98 |
| | | (d, J = 8.1 Hz, 1H), 7.91 |
| | | (d, J = 3.1 Hz, 1H), 7.35 |
| | | (dd, J = 12.3, 1.7 Hz, |
| | | 1H), 6.51 (d, J = 8.1 Hz, |
| | | 1H), 4.30 (s, 3H), 3.52 |
| | | (t, J = 5.7 Hz, 2H), 3.44 |
| | | (t, J = 5.0 Hz, 4H), 3.27 |
| | | (t, J = 6.2 Hz, 2H), 2.83 |
| | | (t, J = 2.7 Hz, 1H), 2.67 |
| | | (t, J = 4.9 Hz, 4H), 2.58 |
| | | (t, J = 5.8 Hz, 2H), 2.35 |
| | | (s, 3H), 2.03 (td, J = 7.4, |
| | | 2.7 Hz, 2H), 1.63 (dt, |
| | | J = 10.3, 6.8 Hz, 4H) |

### Example 137: Synthesis of Compound 378

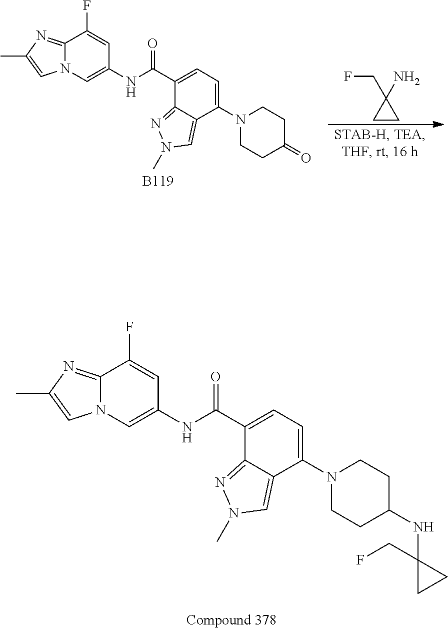 [SMILES: "B119" ; Cc1cn2cc(NC(=O)c3ccc(N4CCC(=O)CC4)c4cn(C)nc34)cc(F)c2n1 ; Cc1cn2cc(NC(=O)c3ccc(N4CCC(NC5(CF)CC5)CC4)c4cn(C)nc34)cc(F)c2n1 ; NC1(CF)CC1 ; "Compound 378"]

To a stirred mixture of N-(8-fluoro-2-methyl-imidazo[1,2-a]pyridin-6-yl)-2-methyl-4-(4-oxo-1-piperidyl)indazole-7-carboxamide (0.1 g, 237.9 μmol) and 1-(fluoromethyl)cyclopropan-1-amine hydrochloride (35 mg, 285.4 μmol) in THF (2 mL) were added TEA (50 mg, 475.7 μmol, 66.3 μL) and NaBH(AcO)₃ (100 mg, 475.7 μmol), and the reaction was stirred for 16 h at room temperature. The resulting mixture was filtered. The filtrate was concentrated under reduced pressure and purified by reversed-phase flash chromatography (Condition 3, Gradient 15) to afford 4-[4-[[1-(fluoromethyl)cyclopropyl]amino]-1-piperidyl]-N-(8-fluoro-2-methyl-imidazo[1,2-a]pyridin-6-yl)-2-methyl-indazole-7-carboxamide (Compound 378, 0.01 g, 20.3 μmol, 9% yield) as a solid. LCMS (ES, m/z): 494 [M+H]⁺. ¹H NMR (400 MHz, Methanol-d₄) δ 9.06 (d, J=1.7 Hz, 1H), 8.46 (s, 1H), 8.05 (d, J=8.1 Hz, 1H), 7.69 (d, J=2.9 Hz, 1H), 7.22 (dd, J=11.9, 1.7 Hz, 1H), 6.50 (d, J=8.2 Hz, 1H), 4.51 (s, 1H), 4.39 (s, 1H), 4.31 (s, 3H), 3.97 (d, J=12.9 Hz, 2H), 3.26-3.16 (m, 1H), 3.05 (t, J=11.8 Hz, 2H), 2.43 (s, 3H), 2.12 (d, J=12.5 Hz, 2H), 1.63 (dd, J=12.0, 3.7 Hz, 1H), 0.82-0.68 (m, 4H).

### Example 138: Synthesis of C114

### Synthesis of C113

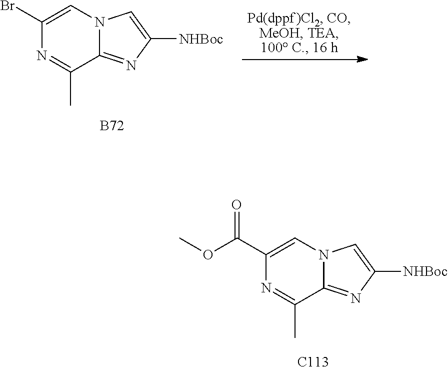 [SMILES: "B72" ; Cc1nc(Br)cn2cc(NC(=O)OC(C)(C)C)nc12 ; COC(=O)c1cn2cc(NC(=O)OC(C)(C)C)nc2c(C)n1 ; "Pd(dppf)Cl2, CO,MeOH, TEA,100° C., 16 h" ; "C113"]

To a solution of tert-butyl N-(6-bromo-8-methyl-imidazo[1,2-a]pyrazin-2-yl)carbamate (1 g, 3.06 mmol) and TEA (927 mg, 9.17 mmol) in MeOH (20 mL) was added Pd(dppf)Cl₂ (223 mg, 305.7 μmol) in a pressure tank. The mixture was purged with nitrogen and then was pressurized to 20 atm with carbon monoxide at 100° C. for 16 hr. The reaction mixture was cooled to room temperature and filtered to remove insoluble solids. The resulting mixture was concentrated under reduced pressure to afford methyl 2-(tert-butoxycarbonylamino)-8-methyl-imidazo[1,2-a]pyrazine-6-carboxylate (850 mg, 2.77 mmol, 91% yield) as a solid. LCMS (ES, m/z): 307 [M+H]⁺.

### Synthesis of C114

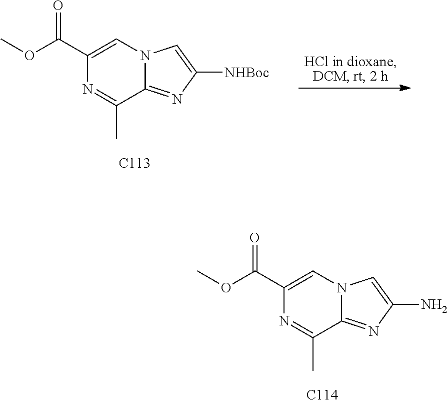 [SMILES: COC(=O)c1cn2cc(NC(=O)OC(C)(C)C)nc2c(C)n1 ; "HCl in dioxane,DCM, rt, 2 h" ; "C113" ; COC(=O)c1cn2cc(N)nc2c(C)n1 ; "C114"]

A solution of methyl 2-(tert-butoxycarbonylamino)-8-methyl-imidazo[1,2-a]pyrazine-6-carboxylate (820 mg, 2.68 mmol) in DCM (8 mL) was treated with HCl (4.0 M in 1,4-dioxane) (4 mL), and the reaction was stirred for 2 h at room temperature under N₂. The resulting mixture was concentrated under reduced pressure to afford methyl 2-amino-8-methyl-imidazo[1,2-a]-pyrazine-6-carboxylate hydrochloride (550 mg, 2.27 mmol, 85% yield) as a solid. LCMS (ES, m/z): 207 [M+H]⁺.

### Example 139: Synthesis of Compound 379

### Synthesis of B121

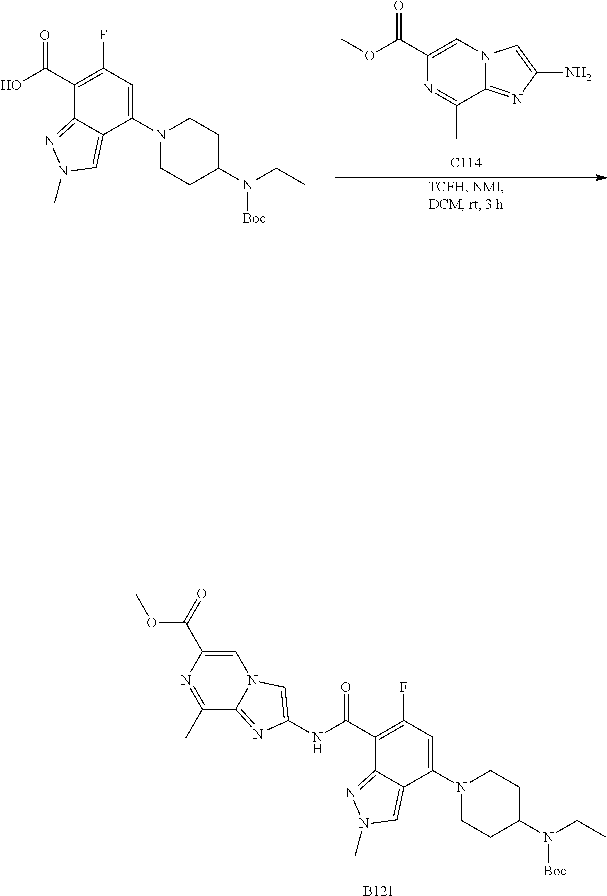

To a stirred mixture of methyl 2-amino-8-methyl-imidazo[1,2-a]pyrazine-6-carboxylate (318 mg, 1.55 mmol) and 4-[4-[tert-butoxycarbonyl(ethyl)amino]-1-piperidyl]-6-fluoro-2-methyl-indazole-7-carboxylic acid (B90-a, 500 mg, 1.19 mmol) in DCM (10 mL) were added NMI (292 mg, 3.57 mmol) and TCFH (500 mg, 1.78 mmol), and the reaction was stirred for 3 h at room temperature. The resulting mixture was diluted with water (40 mL) and extracted with EtOAc (3×50 mL). The combined organic layers were washed with brine (80 mL), dried over anhydrous Na₂SO₄, and filtered. The filtrate was concentrated under reduced pressure and purified by silica gel column chromatography (100% EtOAc) to afford methyl 2-[[4-[4-[tert-butoxycarbonyl(ethyl)amino]-1-piperidyl]-6-fluoro-2-methyl-indazole-7-carbonyl]amino]-8-methyl-imidazo[1,2-a]pyrazine-6-carboxylate (550 mg, 903.6 μmol, 76% yield) as a solid. LCMS (ES, m/z): 609 [M+H]⁺.

### Synthesis of B122

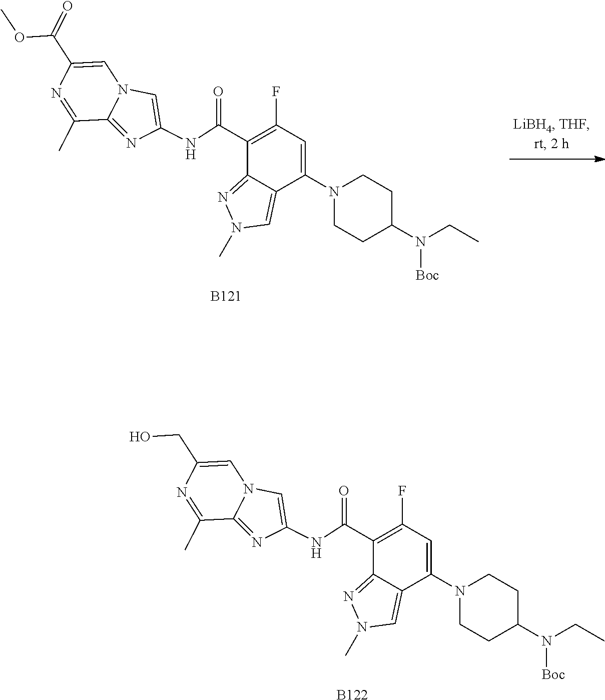 [SMILES: "B121" ; CCN(C(=O)OC(C)(C)C)C1CCN(c2cc(F)c(C(=O)Nc3cn4cc(C(=O)OC)nc(C)c4n3)c3nn(C)cc23)CC1 ; "B122" ; CCN(C(=O)OC(C)(C)C)C1CCN(c2cc(F)c(C(=O)Nc3cn4cc(CO)nc(C)c4n3)c3nn(C)cc23)CC1]

A solution of methyl 2-[[4-[4-[tert-butoxycarbonyl(ethyl)amino]-1-piperidyl]-6-fluoro-2-methyl-indazole-7-carbonyl]amino]-8-methyl-imidazo[1,2-a]pyrazine-6-carboxylate (300 mg, 492.9 μmol) in THF (6 mL) was treated with LiBH₄ (2.0 M solution in THF) (0.74 mL, 1.48 mmol) at 0° C. under N₂. The resulting mixture was stirred for 2 h at room temperature. The reaction was quenched with sat. NH₄Cl (aq.) at 0° C. The resulting mixture was extracted with EtOAc (2×30 mL). The combined organic layers were washed with brine (30 mL), dried over anhydrous Na₂SO₄, and filtered. The filtrate was concentrated under reduced pressure and purified by silica gel column chromatography (50% EtOAc in PE) to afford tert-butyl N-ethyl-N-[1-[6-fluoro-7-[[6-(hydroxymethyl)-8-methyl-imidazo[1,2-a]pyrazin-2-yl]carbamoyl]-2-methyl-indazol-4-yl]-4-piperidyl]carbamate (140 mg, 241.1 μmol, 49% yield) as a solid. LCMS (ES, m/z): 581 [M+H]⁺.

### Synthesis of Compound 379

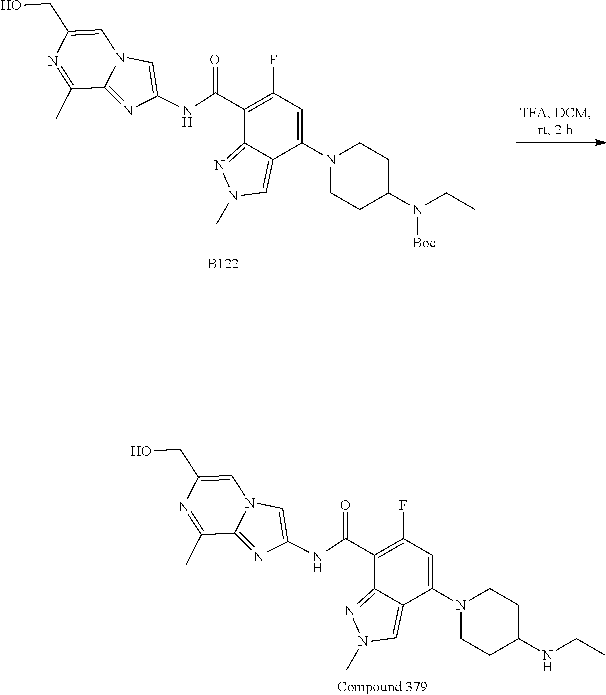

To a stirred mixture of tert-butyl N-ethyl-N-[1-[6-fluoro-7-[[6-(hydroxymethyl)-8-methyl-imidazo[1,2-a]pyrazin-2-yl]carbamoyl]-2-methyl-indazol-4-yl]-4-piperidyl]carbamate (60 mg, 103.3 μmol) in DCM (2 mL) was added TFA (0.5 mL) dropwise, and the reaction was stirred for 2 h at room temperature. The resulting mixture was concentrated under reduced pressure and purified by Prep-HPLC (Condition 5, Gradient 19) to afford 4-[4-(ethylamino)-1-piperidyl]-6-fluoro-N-[6-(hydroxymethyl)-8-methyl-imidazo[1,2-a]pyrazin-2-yl]-2-methyl-indazole-7-carboxamide (Compound 379, 30 mg, 62.4 μmol, 60% yield) as a solid. LCMS (ES, m/z): 481 [M+H]⁺. ¹H NMR (400 MHz, DMSO-d₆) δ 11.57 (s, 1H), 8.82 (s, 1H), 8.41 (d, J=1.5 Hz, 2H), 6.25 (d, J=16.0 Hz, 1H), 5.46 (t, J=5.6 Hz, 1H), 4.55 (dd, J=5.5, 1.2 Hz, 2H), 4.24 (s, 3H), 3.92 (d, J=13.1 Hz, 2H), 3.13 (td, J=13.1, 12.3, 2.8 Hz, 2H), 2.70 (s, 4H), 2.60 (q, J=7.1 Hz, 2H), 2.02-1.85 (m, 2H), 1.41 (q, J=11.5, 9.7 Hz, 2H), 1.04 (t, J=7.1 Hz, 3H).

### Example 140: Synthesis of C117

### Synthesis of C115

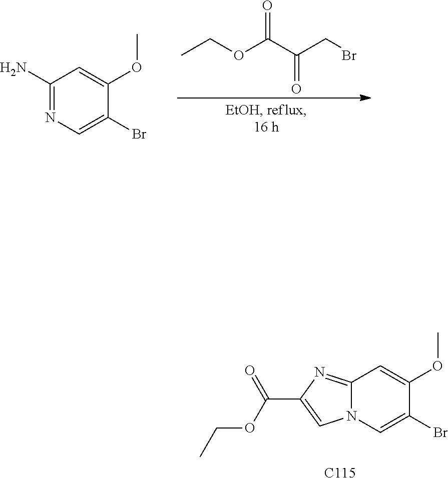

To a mixture of ethyl 3-bromo-2-oxo-propanoate (5.76 g, 29.55 mmol) in EtOH (100 mL) was added 5-bromo-4-methoxy-pyridin-2-amine (5 g, 24.63 mmol). The resulting mixture was stirred for 16 h at 80° C. The mixture was allowed to cool down to room temperature. The precipitated solids were collected by filtration and washed with EtOH (20 mL), to afford ethyl 6-bromo-7-methoxy-imidazo[1,2-a]pyridine-2-carboxylate (2.5 g, 8.36 mmol, 34% yield) as a solid. LCMS (ES, m/z): 299 [M+H]⁺.

### Synthesis of C116

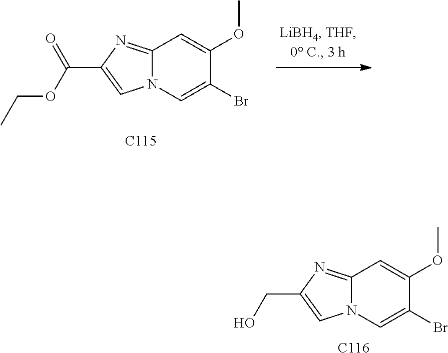 [SMILES: CCOC(=O)c1cn2cc(Br)c(OC)cc2n1 ; "C115" ; COc1cc2nc(CO)cn2cc1Br ; "C116"]

A solution of ethyl 6-bromo-7-methoxy-imidazo[1,2-a]pyridine-2-carboxylate (2 g, 6.69 mmol) in THF (30 mL) was treated with LiBH₄ (2.0 M solution in THF) (6 mL, 183.4 mmol) for 15 min at 0° C. under N₂. The resulting mixture was stirred for 3 h at 0° C. The reaction was quenched by the addition of sat. NH₄Cl (aq.) at 0° C. The resulting mixture was extracted with EtOAc (3×30 mL). The combined organic layers were washed with brine (2×90 mL), dried over anhydrous Na₂SO₄, and filtered. The filtrate was concentrated under reduced pressure and purified by silica gel column chromatography (25% EtOAc in PE) to afford (6-bromo-7-methoxy-imidazo[1,2-a]pyridin-2-yl) methanol (1 g, 3.89 mmol, 58% yield) as a solid. LCMS (ES, m/z): 257 [M+H]⁺.

### Synthesis of C117

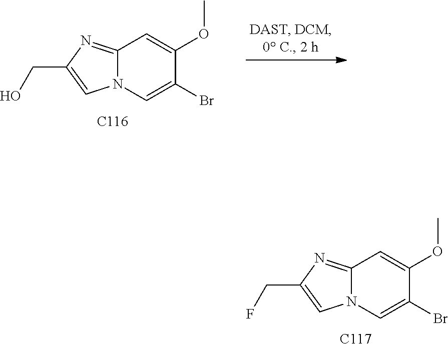

A solution of (6-bromo-7-methoxy-imidazo[1,2-a]pyridin-2-yl) methanol (0.4 g, 1.56 mmol) in DCM (5 mL) was treated with DAST (275.8 mg, 1.71 mmol) for 5 min at 0° C. under N₂, followed by an additional 2 h at 0° C. The reaction was quenched by the addition of sat. NaHCO₃ (aq.) at 0° C. and extracted with DCM (3×10 mL). The combined organic layers were washed with brine (2×30 mL), dried over anhydrous Na₂SO₄, and filtered. The filtrate was concentrated under reduced pressure and purified by silica gel column chromatography (17% EtOAc in PE) to afford 6-bromo-2-(fluoromethyl)-7-methoxy-imidazo[1,2-a]pyridine (160 mg, 617.6 μmol, 40% yield) as a solid. LCMS (ES, m/z): 259 [M+H]⁺.

### Example 141: Synthesis of C119

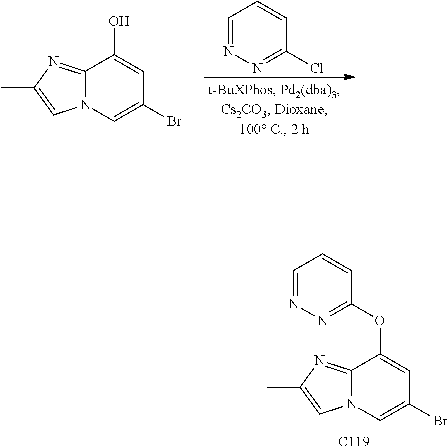 [SMILES: Cc1cn2cc(Br)cc(O)c2n1 ; Clc1cccnn1 ; Cc1cn2cc(Br)cc(Oc3cccnn3)c2n1 ; "C119"]

To a stirred mixture of 6-bromo-2-methyl-imidazo[1,2-a]pyridin-8-ol (0.15 g, 660.6 μmol) and 3-chloropyridazine (90.8 mg, 792.8 μmol) in Dioxane (3 mL) were added Cs₂CO₃ (645.7 mg, 1.98 mmol), t-BuXPhos (90.7 mg, 132.1 μmol) and Pd₂(dba)₃ (60.4 mg, 66.1 μmol) at room temperature under N₂. The resulting mixture was stirred for 2 h at 100° C. under N₂. The mixture was allowed to cool down to room temperature. The resulting mixture was concentrated under reduced pressure and purified by silica gel column chromatography (75% EtOAc in PE) to afford 6-bromo-2-methyl-8-pyridazin-3-yloxy-imidazo[1,2-a]pyridine (0.13 g, 426.1 μmol, 65% yield) as a solid. LCMS (ES, m/z): 305 [M+H]⁺.

An analogous method was followed to obtain the following compounds.

| Compound | Starting Material | Characterization |
|---|---|---|
| 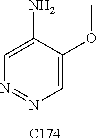 | 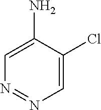 | Modification: The reaction was ran in MeOH for 2 h at 90° C. The residue was purified by silica gel column chromatography (25% EtOAc in PE) to afford 5- methoxypyridazin-4-amine (C174, 0.14 g, 1.12 mmol, 97% yield) as a solid. LCMS (ES, m/z): 126 [M + H]⁺. |
| | Only | |

### Example 142: Synthesis of Compound 412

### Synthesis of B123

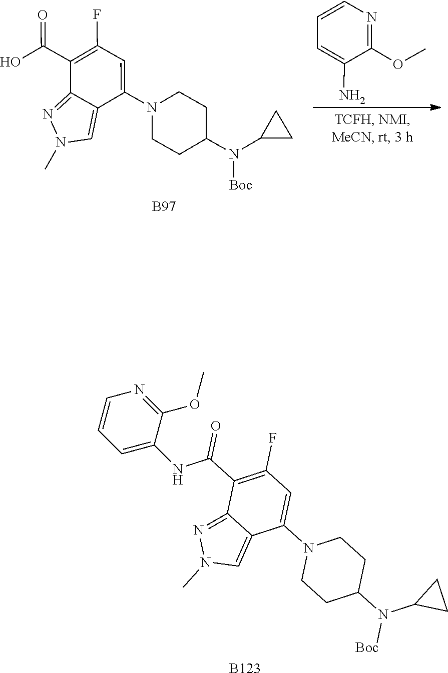

To a stirred mixture of 4-[4-[tert-butoxycarbonyl(cyclopropyl)amino]-1-piperidyl]-6-fluoro-2-methyl-indazole-7-carboxylic acid (0.13 g, 300.6 μmol) and 2-methoxypyridin-3-amine (45 mg, 360.7 μmol) in MeCN (2 mL) were added TCFH (127 mg, 450.9 μmol) and NMI (74 mg, 901.8 μmol, 71.5 μL), and the reaction was stirred for 3 h at room temperature. The resulting mixture was diluted with water and the precipitate was collected by filtration. The solid was purified by trituration with MTBE (3 mL). The resulting solid was dried to afford tert-butyl N-cyclopropyl-N-[1-[6-fluoro-7-[(2-methoxy-3-pyridyl)carbamoyl]-2-methyl-indazol-4-yl]-4-piperidyl]carbamate (0.15 g, 278.5 μmol, 93% yield) as a solid. LCMS (ES, m/z): 539 [M+H]⁺.

### Synthesis of Compound 412

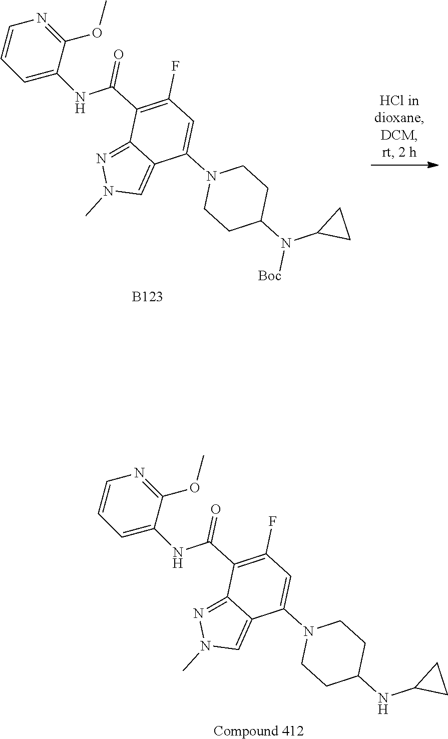 [SMILES: "B123" ; COc1ncccc1NC(=O)c1c(F)cc(N2CCC(N(C(=O)OC(C)(C)C)C3CC3)CC2)c2cn(C)nc12 ; COc1ncccc1NC(=O)c1c(F)cc(N2CCC(NC3CC3)CC2)c2cn(C)nc12 ; "Compound 412"]

To a solution of tert-butyl cyclopropyl(1-(6-fluoro-7-((2-methoxypyridin-3-yl)carbamoyl)-2-methyl-2H-indazol-4-yl)piperidin-4-yl)carbamate (0.06 g, 111.4 μmol) in DCM (2 mL) was added HCl (4 M in dioxane, 1 mL). The reaction was stirred for 2 h at rt. The resulting mixture was concentrated under reduced pressure and adjusted to pH 8 with saturated NaHCO₃ (aq.). The precipitate was collected by filtration. The solid was purified by trituration with MTBE (3 mL). The resulting solid was dried to afford 4-(4-(cyclopropylamino)piperidin-1-yl)-6-fluoro-N-(2-methoxypyridin-3-yl)-2-methyl-2H-indazole-7-carboxamide (0.03 g, 68.42 μmol, 61% yield) as a solid. LCMS (ES, m/z): 439 [M+H]⁺. ¹H NMR (400 MHz, DMSO-d₆) δ 11.63 (s, 1H), 8.85 (s, 1H), 8.76 (dd, J=7.8, 1.8 Hz, 1H), 7.83 (dd, J=5.0, 1.7 Hz, 1H), 6.99 (dd, J=7.8, 5.0 Hz, 1H), 6.26 (d, J=16.0 Hz, 1H), 4.23 (s, 3H), 4.06 (s, 3H), 4.00 (d, J=13.2 Hz, 2H), 3.19-3.05 (m, 3H), 2.44 (s, 1H), 2.10 (d, J=12.3 Hz, 2H), 1.60 (d, J=11.9 Hz, 2H), 0.61 (d, J=8.1 Hz, 4H).

An analogous method was followed to obtain the following compounds.

| Compound | Starting Material | Characterization |
|---|---|---|
| 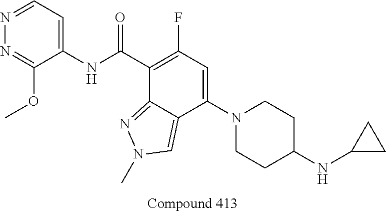 [SMILES: COc1nnccc1NC(=O)c1c(F)cc(N2CCC(NC3CC3)CC2)c2cn(C)nc12] | 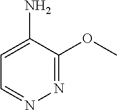 | Modification: Step 1 was ran at 60° C. Step 2 was ran using TFA in DCM for 1 h at room temperature. The resulting mixture was purified by reversed-phase flash chromatography (Condition 3, Gradient 22) to afford 4- [4-(cyclopropylamino)-1-piperidyl]-6- fluoro-N-(3-methoxypyridazin-4-yl)-2- methyl-indazole-7-carboxamide (Compound 413, 25 mg, 56.9 μmol, 44% yield) as a solid. LCMS (ES, m/z): 440 [M + H]⁺. ¹H NMR (400 MHz, DMSO-d₆) δ 11.98 (s, 1H), 8.86 (s, 1H), |
| | | 8.72 (d, J = 5.3 Hz, 1H), 8.45 (d, J = 5.3 |
| | | Hz, 1H), 6.25 (d, J = 16.5 Hz, 1H), 4.23 |
| | | (d, J = 5.9 Hz, 6H), 3.96 (d, J = 13.2 Hz, |
| | | 2H), 3.19 (ddd, J = 13.5, 11.1, 2.8 Hz, |
| | | 2H), 2.83 (dt, J = 9.7, 5.4 Hz, 1H), 2.12 |
| | | (tt, J = 6.8, 3.6 Hz, 1H), 2.03-1.95 (m, |
| | | 2H), 1.44 (q, J = 11.3, 9.6 Hz, 2H), |
| | | 0.45-0.34 (m, 2H), 0.27-0.19 (m, 2H). |
| 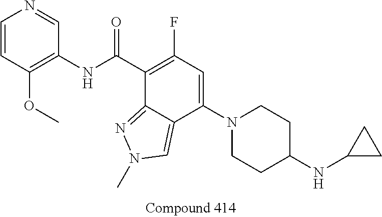 [SMILES: COc1ccncc1NC(=O)c1c(F)cc(N2CCC(NC3CC3)CC2)c2cn(C)nc12] | 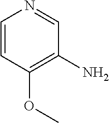 | Modification: Step 2 was ran using TFA in DCM for 2 h at room temperature. The resulting mixture was purified by reversed-phase flash chromatography (Condition 3, Gradient 10) to afford 4- [4-(cyclopropylamino)-1-piperidyl]-6- fluoro-N-(4-methoxy-3-pyridyl)-2- methyl-indazole-7-carboxamide (Compound 414, 23.7 mg, 54 μmol, 22% yield) as a solid. LCMS (ES, m/z): 439 [M + H]⁺. ¹H NMR (400 MHz, DMSO-d₆) δ 11.52 (s, 1H), 9.57 (s, 1H), 8.80 (s, 1H), 8.21 (d, J = 5.4 Hz, 1H), 7.15 (d, J = 5.5 Hz, 1H), 6.22 (d, J = |
| | | 16.2 Hz, 1H), 4.22 (s, 3H), 4.04 (s, 3H), |
| | | 3.91 (d, J = 13.1 Hz, 2H), 3.18-3.07 (m, |
| | | 2H), 2.80 (dt, J = 10.0, 5.4 Hz, 1H), 2.26 |
| | | (s, 1H), 2.11 (tq, J = 6.5, 3.2 Hz, 1H), |
| | | 1.99 (dd, J = 13.3, 3.7 Hz, 2H), 1.46- |
| | | 1.43 (m, 2H), 0.39 (dt, J = 6.1, 3.0 Hz, |
| | | 2H), 0.27-0.19 (m, 2H). |
| 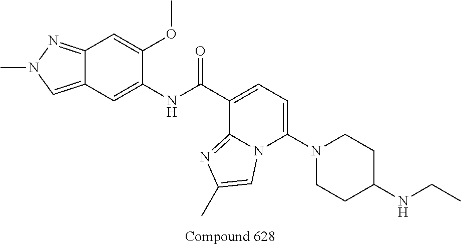 | 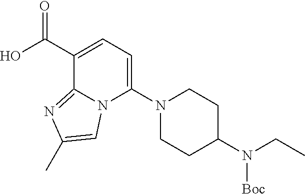 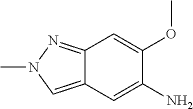 | The residue was purified by reversed- phase flash chromatography (Condition 3, Gradient 20) to afford 5-[4- (ethylamino)-1-piperidyl]-N-(6- methoxy-2-methyl-indazol-5-yl)-2- methyl-imidazo[1,2-a]pyridine-8- carboxamide (Compound 628, 0.055 g, 119.2 umol, 37% yield) as a solid. LCMS (ES, m/z): [M + H]⁺. ¹H NMR (300 MHz, DMSO-d₆) δ 12.82 (s, 1H), 8.83 (s, 1H), 8.21 (s, 1H), 8.10 (d, J = 7.8 Hz, 1H), 7.58 (s, 1H), 7.08 (s, 1H), 6.60 (d, J = 7.9 Hz, 1H), 4.08 (s, 3H), 4.06 (s, 3H), 3.49 (d, J = 11.7 Hz, 2H), 2.95-2.82 (m, 2H), 2.62 (s, 2H), 2.00 (d, J = 12.6 Hz, 2H), 1.55 (d, J = 11.7 Hz, 3H), 1.05 (t, J = 7.0 Hz, 3H). |
| 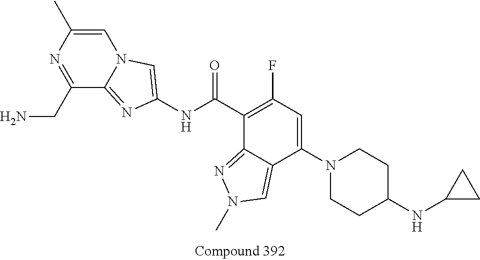 [SMILES: Cc1cn2cc(NC(=O)c3c(F)cc(N4CCC(NC5CC5)CC4)c4cn(C)nc34)nc2c(CN)n1] | 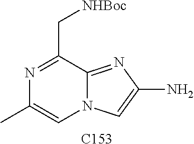 | Modification: Step 1 was ran for 16 h at room temperature. The crude product was purified by Prep- HPLC (Condition 5, Gradient 10a) to afford N-[8-(aminomethyl)-6-methyl- imidazo[1,2-a]-pyrazin-2-yl]-4-[4- (cyclopropylamino)-1-piperidyl]-6- fluoro-2-methyl-indazole-7- carboxamide (Compound 392, 19.9 mg, 40.5 μmol, 31% yield) as a solid. LCMS (ES, m/z): 492 [M + H]⁺. ¹H NMR (300 MHz, DMSO-d₆) δ 11.58 (s, 1H), 8.82 (s, 1H), 8.34 (d, J = 11.8 Hz, 2H), 6.25 (d, J = 16.0 Hz, 1H), 4.24 (s, 3H), 4.12 (s, 2H), 3.92 (d, J = 13.0 Hz, 3H), 3.13 (t, J = 11.8 Hz, 2H), 2.77 (d, |
| | | J = 20.9 Hz, 1H), 2.42 (s, 3H), 2.11 (dq, |
| | | J = 7.0, 3.5 Hz, 1H), 1.99 (d, J = 12.9 |
| | | Hz, 2H), 1.45 (d, J = 11.4 Hz, 2H), 0.39 |
| | | (dt, J = 6.2, 3.0 Hz, 2H), 0.23 (p, J = 4.0 |
| | | Hz, 2H). |
| 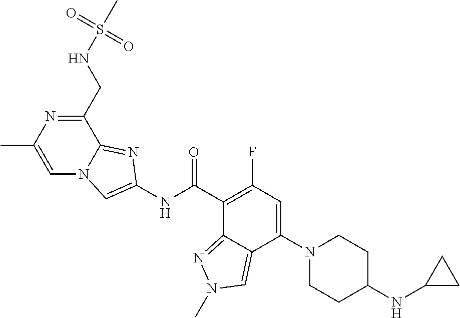 [SMILES: Cc1cn2cc(NC(=O)c3c(F)cc(N4CCC(NC5CC5)CC4)c4cn(C)nc34)nc2c(CNS(C)(=O)=O)n1] | 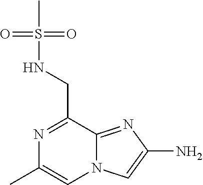 C154 | Modification: Step 1 was ran for 16 h. Step 2 was ran using TFA in DCM for 2 h at room temperature. The resulting mixture were concentrated under reduced pressure and lyophilized to afford 4-(4- (cyclopropylamino)piperidin-1-yl)-6- fluoro-2-methyl-N-(6-methyl-8- (methylsulfonamidomethyl)imidazo[1,2- a]pyrazin-2-yl)-2H-indazole-7- carboxamide 2,2,2-trifluoroacetate (Compound 393, 6 mg, 8.8 μmol, 39% yield) as a solid. LCMS (ES, m/z): 570 [M + H]⁺. ¹H NMR (300 MHz, DMSO- d₆) δ 11.63 (s, 1H), 8.89 (s, 1H), 8.73 (s, 2H), 8.44 (d, J = 1.1 Hz, 1H), 8.37 (s, 1H), 7.50 (t, J = 5.8 Hz, 1H), 6.36 (d, J = 15.6 Hz, 1H), 4.66 (d, J = 5.7 Hz, 2H), 4.25 (s, 3H), 4.10 (d, J = 13.3 Hz, 2H), 3.54-3.52 (m, 2H), 3.14 (d, J = 12.8 Hz, |
| Compound 393 | | 2H), 3.06 (s, 3H), 2.80-2.75 (m, 1H), |
| | | 2.45 (s, 3H), 2.19 (d, J = 12.2 Hz, 2H), |
| | | 1.80-1.65 (m, 2H), 0.84-0.81 (m, 4H). |
| 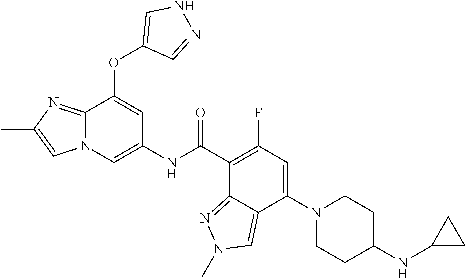 [SMILES: Cc1cn2cc(NC(=O)c3c(F)cc(N4CCC(NC5CC5)CC4)c4cn(C)nc34)cc(Oc3cn[nH]c3)c2n1] | 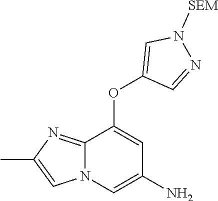 C164 | The crude product was purified by Prep- HPLC (Condition 16, Gradient 1) to afford N-(8-((1H-pyrazol-4-yl)oxy)-2- methylimidazo[1,2-a]-pyridin-6-yl)-4- (4-(cyclopropylamino)piperidin-1-yl)- 6-fluoro-2-methyl-2H-indazole-7- carboxamide 2,2,2-trifluoroacetate (Compound 410, 10 mg, 15.2 μmol, 29% yield) as a solid. LCMS (ES, m/z): 544 [M + H]⁺. ¹H NMR (400 MHz, DMSO-d₆) δ 10.95 (s, 1H), 8.97 (s, 1H), 8.78 (d, J = 4.3 Hz, 4H), 7.98 (d, J = 16.9 Hz, 2H), 7.55 (s, 1H), 6.30 (d, J = 14.8 Hz, 1H), 4.19 (s, 3H), 4.01 (d, J = 13.0 Hz, 2H), 3.46 (s, 1H), 3.03 (t, J = 12.3 Hz, 2H), 2.77 (s, 1H), 2.44 (s, 3H), 2.17 (d, J = 11.8 Hz, 2H), 1.81-1.60 (m, |
| Compound 410 | | 2H), 0.82 (dd, J = 7.4, 2.7 Hz, 4H). |
| 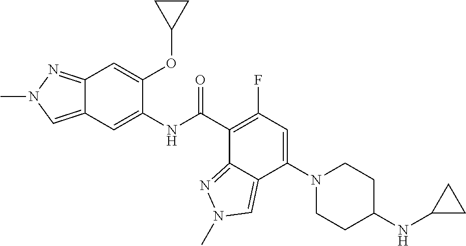 [SMILES: Cn1cc2cc(NC(=O)c3c(F)cc(N4CCC(NC5CC5)CC4)c4cn(C)nc34)c(OC3CC3)cc2n1] | 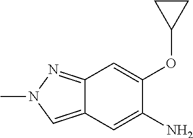 C188 | The crude product was purified by Prep- HPLC (Condition 5, Gradient 12) to afford N-[6-(cyclopropoxy)-2-methyl- indazol-5-yl]-4-[4-(cyclopropylamino)- 1-piperidyl]-6-fluoro-2-methyl- indazole-7-carboxamide (Compound 438, 15 mg, 29 μmol, 30% yield) as a solid. LCMS (ES, m/z): 518 [M + H]⁺. ¹H NMR (400 MHz, DMSO-d₆) δ 10.29 (s, 1H), 8.74 (s, 1H), 8.28 (s, 1H), 7.59 (d, J = 9.1 Hz, 1H), 7.24 (d, J = 9.1 Hz, 1H), 6.19 (d, J = 15.8 Hz, 1H), 4.13 (s, 3H), 4.07 (s, 3H), 3.98 (tt, J = 6.0, 3.6 Hz, 1H), 3.85 (d, J= 13.0 Hz, 2H), 3.11- 3.00 (m, 2H), 2.77 (dt, J = 9.9, 5.5 Hz, |
| Compound 438 | | 1H), 2.15-2.03 (m, 1H), 1.98 (d, J = |
| | | 12.1 Hz, 2H), 1.52-1.38 (m, 2H), 0.71- |
| | | 0.64 (m, 4H), 0.43-0.34 (m, 2H), 0.26- |
| | | 0.18 (m, 2H). |
| 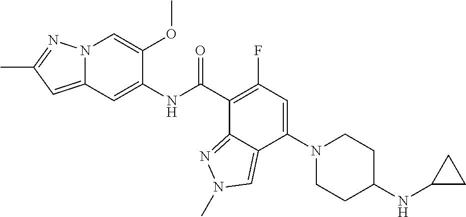 [SMILES: COc1cn2nc(C)cc2cc1NC(=O)c1c(F)cc(N2CCC(NC3CC3)CC2)c2cn(C)nc12] | 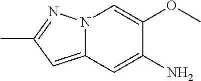 C197 | Modification: Step 1 was ran for 6 h at 40° C. Step 2 was ran at room temperature for 2 h. The crude product was purified by Prep- HPLC (Condition 5, Gradient 20) to afford 4-[4-(cyclopropylamino)-1- piperidyl]-6-fluoro-N-(6-methoxy-2- methyl-pyrazolo[1,5-a]pyridin-5-yl)-2- methyl-indazole-7-carboxamide (Compound 432, 25 mg, 50.9 μmol, 38% yield) as a solid. LCMS (ES, m/z): 492 [M + H]⁺. ¹H NMR (300 MHz, DMSO-d₆) δ 11.77 (s, 1H), 8.80 (s, 1H), 8.58 (s, 1H), 8.32 (s, 1H), 6.27-6.14 (m, |
| Compound 432 | | 2H), 4.21 (s, 3H), 4.00 (s, 3H), 3.90 (d, |
| | | J = 13.1 Hz, 2H), 3.12 (t, J = 12.0 Hz, |
| | | 2H), 2.79 (q, J = 8.5, 7.3 Hz, 1H), 2.31 |
| | | (s, 4H), 2.09 (dq, J = 6.7, 3.4 Hz, 1H), |
| | | 2.03-1.87 (m, 2H), 1.42 (q, J = 11.3 Hz, |
| | | 2H), 0.37 (dt, J = 6.2, 3.0 Hz, 2H), 0.21 |
| | | (p, J = 3.9 Hz, 2H). |
| 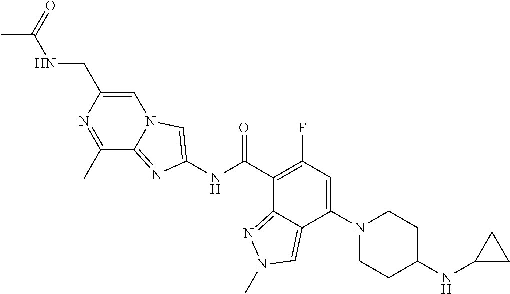 [SMILES: CC(=O)NCc1cn2cc(NC(=O)c3c(F)cc(N4CCC(NC5CC5)CC4)c4cn(C)nc34)nc2c(C)n1] | 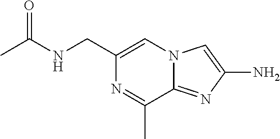 C199 | Modification: Step 1 was ran for 16 h at 60° C. Step 2 was ran using TFA in DCM for 1 h at room temperature. The crude product was purified by Prep- HPLC (Condition 5, Gradient 11) to afford N-[6-(acetamidomethyl)-8- methyl-imidazo [1,2-a]pyrazin-2-yl]-4- [4-(cyclopropylamino)-1-piperidyl]-6- fluoro-2-methyl-indazole-7- carboxamide (Compound 424, 3.5 mg, 6.6 μmol, 10% yield) as a solid. LCMS (ES, m/z): 534 [M + H]⁺. ¹H NMR (400 MHz, DMSO-d₆) δ 11.59 (s, 1H), 8.83 (s, 1H), 8.43 (t, J = 5.8 Hz, 1H), 8.38 (d, J = 3.6 Hz, 2H), 6.25 (d, J = 16.0 Hz, 1H), 4.31-4.22 (m, 5H), 3.92 (d, J = 13.1 Hz, 2H), 3.14 (t, J = 12.0 Hz, 2H), 2.80 (s, 1H), 2.72 (s, 3H), 2.27 (s, 1H), |
| Compound 424 | | 2.12 (s, 1H), 1.99 (d, J = 12.4 Hz, 2H), |
| | | 1.91 (s, 3H), 1.45 (q, J = 9.8 Hz, 2H), |
| | | 0.45-0.34 (m, 2H), 0.27-0.19 (m, 2H). |
| 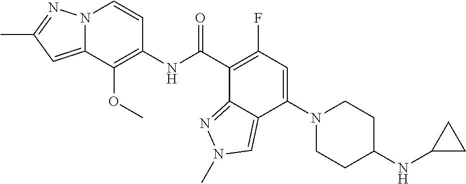 | 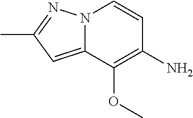 C211 | Modification: Step 1 was ran for 2 h Step 2 was ran using TFA in DCM at room temperature for 2 h. The crude product was purified by Prep- HPLC (Condition 2, Gradient 9) to afford 4-(4-(cyclopropylamino)- piperidin-1-yl)-6-fluoro-N-(4- methoxy-2-methylpyrazolo[1,5-a]- pyridin-5-yl)-2-methyl-2H-indazole-7- carboxamide 2,2,2-trifluoroacetate (Compound 447, 30 mg, 49.5 μmol, 37% yield) as a solid. LCMS (ES, m/z): |
| Compound 447 | | 492 [M + H]⁺. ¹H NMR (400 MHz, |
| | | DMSO-d₆) δ 11.64 (s, 1H), 8.90 (s, 1H), |
| | | 8.86 (s, 2H), 8.36 (d, J = 7.6 Hz, 1H), |
| | | 8.09 (d, J = 7.6 Hz, 1H), 6.46 (s, 1H), |
| | | 6.34 (d, J = 15.9 Hz, 1H), 4.26 (s, 3H), |
| | | 4.12-4.10 (m, 2H), 4.09 (s, 3H), 3.51 (s, |
| | | 1H), 3.12 (t, J = 12.6 Hz, 2H), 2.86-2.71 |
| | | (m, 1H), 2.39 (s, 3H), 2.20 (d, J = 12.3 |
| | | Hz, 2H), 1.72 (td, J = 13.5, 13.0, 9.6 Hz, |
| | | 2H), 0.85 (s, 2H), 0.83 (s, 2H). |

### Example 143: Synthesis of Compound 411

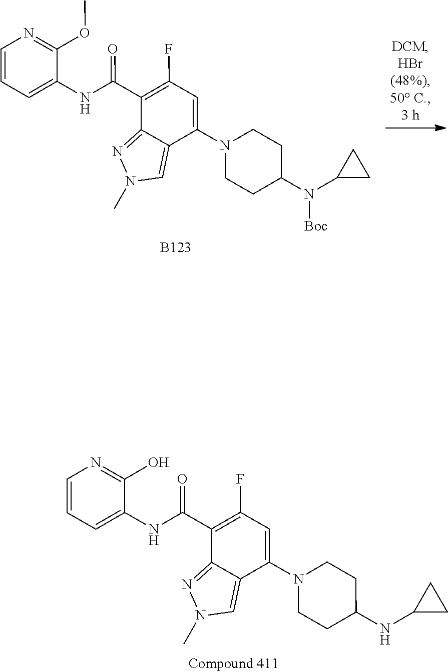 [SMILES: "B123" ; COc1ncccc1NC(=O)c1c(F)cc(N2CCC(N(C(=O)OC(C)(C)C)C3CC3)CC2)c2cn(C)nc12 ; Cn1cc2c(N3CCC(NC4CC4)CC3)cc(F)c(C(=O)Nc3cccnc3O)c2n1]

To a solution of tert-butyl N-cyclopropyl-N-[1-[6-fluoro-7-[(2-methoxy-3-pyridyl)-carbamoyl]-2-methyl-indazol-4-yl]-4-piperidyl]carbamate (0.07 g, 130 μmol) in DCM (2 mL) was added HBr (48 wt. % in water, 210 mg, 2.60 mmol), and the reaction was stirred for 3 h at 50° C. The resulting mixture was concentrated under reduced pressure, adjusted to pH 8 with saturated NaHCO₃ (aq.), and filtered. The filtrate was concentrated under reduced pressure and purified by reversed-phase flash chromatography (Condition 3, Gradient 4) to afford 4-[4-(cyclopropylamino)-1-piperidyl]-6-fluoro-N-(2-hydroxy-3-pyridyl)-2-methyl-indazole-7-carboxamide (Compound 411, 0.015 g, 35.3 μmol, 27% yield) as a solid. LCMS (ES, m/z): 425 [M+H]⁺. ¹H NMR (400 MHz, DMSO-d₆) δ 11.77 (s, 1H), 11.46 (s, 1H), 8.76 (s, 1H), 8.48 (dd, J=7.4, 1.9 Hz, 1H), 7.07 (dd, J=6.6, 1.9 Hz, 1H), 6.27-6.15 (m, 2H), 4.17 (s, 3H), 3.90 (d, J=13.0 Hz, 2H), 3.10 (td, J=12.8, 12.0, 2.7 Hz, 2H), 2.79 (dt, J=10.0, 5.7 Hz, 1H), 2.28 (s, 1H), 2.11 (tt, J=6.7, 3.6 Hz, 1H), 2.02-1.94 (m, 2H), 1.51-1.42 (m, 1H), 0.40 (td, J=6.4, 4.0 Hz, 2H), 0.27-0.19 (m, 2H).

An analogous method was followed to obtain the following compounds.

| Compound | Starting Material | Characterization |
|---|---|---|
| 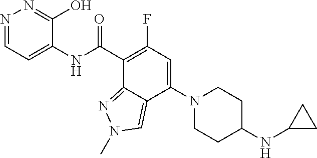 Compound 420 | 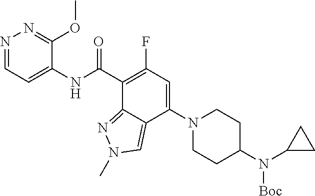 [in route to Compound 413] | Modification: The reaction was ran in H₂O, at 100° C. for 8 h. The residue was purified by reversed-phase flash chromatography (Condition 3, Gradient 24) to afford 4- [4-(cyclopropylamino)-1- piperidyl]-6-fluoro-N-(3- hydroxypyridazin-4-yl)-2- methyl-indazole-7- carboxamide (Compound 420, 8 mg, 18.8 μmol, 16% yield) as a solid. LCMS |
| | | (ES, m/z): 426 [M + H]⁺. ¹H |
| | | NMR (400 MHz, DMSO- |
| | | d₆) δ 13.14 (s, 1H), 11.99 (s, |
| | | 1H), 8.82 (s, 1H), 8.17 (d, J = |
| | | 4.7 Hz, 1H), 7.85 (d, J = |
| | | 4.7 Hz, 1H), 6.23 (d, J = |
| | | 16.4 Hz, 1H), 4.18 (s, 3H), |
| | | 3.95 (d, J = 13.2 Hz, 2H), |
| | | 3.18 (t, J = 11.9 Hz, 2H), |
| | | 2.81 (s, 1H), 2.27 (s, 1H), |
| | | 2.11 (dq, J = 6.6, 3.4 Hz, |
| | | 1H), 1.98 (d, J = 12.8 Hz, |
| | | 2H), 1.50-1.37 (m, 2H), |
| | | 0.44-0.35 (m, 2H), 0.23 (q, |
| | | J = 3.3, 2.7 Hz, 2H). |

### Example 144: Synthesis of C122

### Synthesis of C121

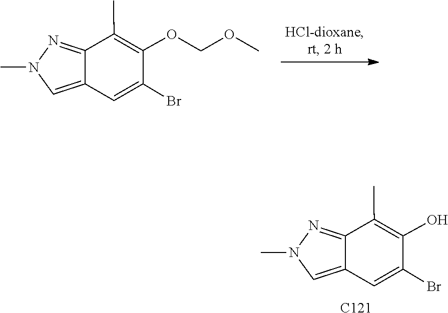

To a stirred mixture of 5-bromo-6-(methoxymethoxy)-2,7-dimethyl-indazole (500 mg, 1.75 mmol) in DCM (10 mL) was added HCl-dioxane (4 M) (2 mL) dropwise, and the reaction was stirred for 2 h at room temperature. The resulting mixture was concentrated under reduced pressure and diluted with water (2 mL). The mixture was basified to pH 8 with saturated NaHCO₃ (aq.) and extracted with DCM (3×10 mL). The combined organic layers were washed with water (2×20 mL) and brine (20 mL), dried over anhydrous Na₂SO₄, and filtered. The filtrate was concentrated under reduced pressure, to afford 5-bromo-2,7-dimethyl-indazol-6-ol (C121, 400 mg, 1.66 mmol, 95% yield) as a solid. LCMS (ES, m/z): 241 [M+H]⁺.

### Synthesis of C122

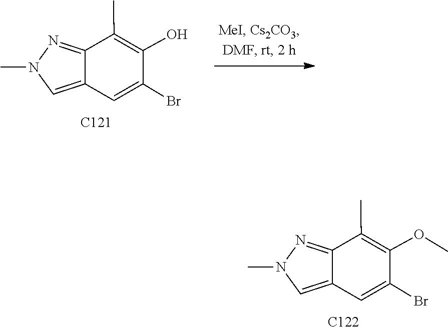

To a stirred mixture of 5-bromo-2,7-dimethyl-indazol-6-ol (400 mg, 1.66 mmol) in DMF (8 mL) were added Cs₂CO₃ (2.16 g, 6.64 mmol) and Mel (471.2 mg, 3.32 mmol), and the reaction was stirred for 2 h at room temperature. The resulting mixture was diluted with water (20 mL) and extracted with EtOAc (2×20 mL). The combined organic layer was washed with water (2×30 mL), dried by anhydrous Na₂SO₄, and filtered. The filtrate was concentrated under reduced pressure and purified by silica gel column chromatography (67% EtOAc in PE) to afford 5-bromo-6-methoxy-2,7-dimethyl-indazole (C122, 380 mg, 1.49 mmol, 90% yield) as a solid. LCMS (ES, m/z): 255 [M+H]⁺.

### Example 145: Synthesis of C125

### Synthesis of C123

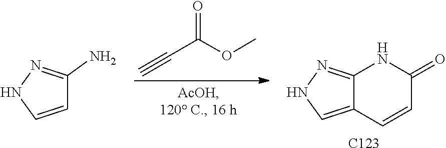

To a solution of 1H-pyrazol-3-amine (10 g, 120.4 mmol) in AcOH (100 mL) was added methyl prop-2-ynoate (10.12 g, 120.4 mmol, 10.71 mL). The mixture was stirred for 16 h at 120° C. The resulting mixture was concentrated under reduced pressure and acidified to pH 8 with saturated NaHCO₃ (aq.). The precipitated solids were collected by filtration and purified by trituration with DCM (10 mL) to afford 2,7-dihydropyrazolo[3,4-b]pyridin-6-one (5.7 g, 42.2 mmol, 35% yield) as a solid. LCMS (ES, m/z): 136 [M+H]⁺.

### Synthesis of C124

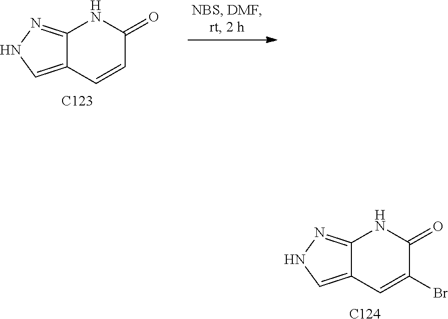 [SMILES: O=c1ccc2c[nH]nc2[nH]1 ; "C123" ; O=c1[nH]c2n[nH]cc2cc1Br ; "C124"]

To a solution of 2,7-dihydropyrazolo[3,4-b]pyridin-6-one (5.6 g, 41.44 mmol) in DMF (8 mL) was added NBS (7.38 g, 41.44 mmol), and the reaction was stirred for 2 h at rt. The reaction was diluted with H₂O (30 mL) and the precipitated solids were collected by filtration and purified by trituration with MTBE (10 mL) to afford 5-bromo-2,7-dihydropyrazolo[3,4-b]pyridin-6-one (6 g, 28 mmol, 68% yield) as a solid. ¹H NMR (400 MHz, DMSO-d₆) δ 7.80 (d, J=1.9 Hz, 1H), 7.45 (d, J=2.1 Hz, 1H).

### Synthesis of C125

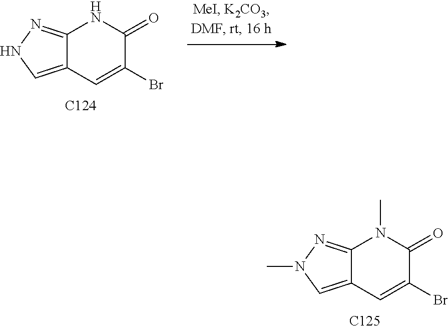

To a solution of 5-bromo-2,7-dihydropyrazolo[3,4-b]pyridin-6-one (2 g, 9.34 mmol) in DMF (20 mL) were added K₂CO₃ (3.23 g, 23.36 mmol) and Mel (2.92 g, 20.56 mmol, 1.28 mL), and the reaction was stirred for 16 h at rt. The reaction was diluted with H₂O (100 mL) and extracted with DCM (2×100 mL). The combined organic layers were washed with H₂O (2×100 mL), dried over anhydrous Na₂SO₄, and filtered. The filtrate was concentrated under reduced pressure and purified by silica gel column chromatography (40% EtOAc in DCM) to afford 5-bromo-2,7-dimethyl-pyrazolo[3,4-b]pyridin-6-one (1 g, 4.1 mmol, 44% yield) as an oil. LCMS (ES, m/z): 242 [M+H]⁺.

### Example 146: Synthesis of Compound 435

### Synthesis of B124

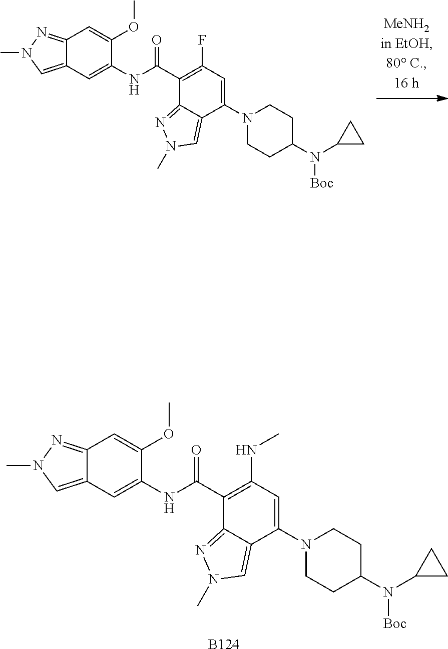 [SMILES: COc1cc2nn(C)cc2cc1NC(=O)c1c(F)cc(N2CCC(N(C(=O)OC(C)(C)C)C3CC3)CC2)c2cn(C)nc12 ; "B124" ; CNc1cc(N2CCC(N(C(=O)OC(C)(C)C)C3CC3)CC2)c2cn(C)nc2c1C(=O)Nc1cc2cn(C)nc2cc1OC]

To a stirred mixture of tert-butyl N-cyclopropyl-N-[1-[6-fluoro-7-[(6-methoxy-2-methyl-indazol-5-yl)carbamoyl]-2-methyl-indazol-4-yl]-4-piperidyl]carbamate (100 mg, 169.0 μmol) in MeNH₂ (2 mL, 2 M in EtOH), and the reaction was stirred for 16 h at 80° C. The resulting mixture was concentrated under reduced pressure and purified by silica gel column chromatography (80% EtOAc in PE) to afford tert-butyl N-cyclopropyl-N-[1-[7-[(6-methoxy-2-methyl-indazol-5-yl)carbamoyl]-2-methyl-6-(methylamino) indazol-4-yl]-4-piperidyl]carbamate (70 mg, 116.1 μmol, 69% yield) as a solid. LCMS (ES, m/z): 603 [M+H]⁺.

### Synthesis of Compound 435

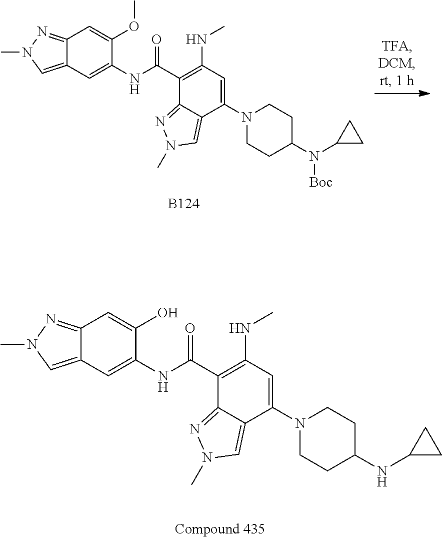 [SMILES: "B124" ; CNc1cc(N2CCC(NC3CC3)CC2)c2cn(C)nc2c1C(=O)Nc1cc2cn(C)nc2cc1O ; CNc1cc(N2CCC(N(C(=O)OC(C)(C)C)C3CC3)CC2)c2cn(C)nc2c1C(=O)Nc1cc2cn(C)nc2cc1OC ; "Compound 435"]

To a stirred solution of tert-butyl N-cyclopropyl-N-[1-[7-[(6-methoxy-2-methyl-indazol-5-yl)carbamoyl]-2-methyl-6-(methylamino) indazol-4-yl]-4-piperidyl]carbamate (70 mg, 116.1 μmol) in DCM (2 mL) was added TFA (1 mL) at rt. The resulting mixture was stirred for 1 h. The resulting mixture was concentrated under reduced pressure and purified by Prep-HPLC (Condition 2, Gradient 10) to afford 4-(4-(cyclopropylamino)piperidin-1-yl)-N-(6-hydroxy-2-methyl-2H-indazol-5-yl)-2-methyl-6-(methylamino)-2H-indazole-7-carboxamide 2,2,2-trifluoroacetate (Compound 435, 31 mg, 61.7 μmol, 53% yield) as a solid. LCMS (ES, m/z): 503 [M+H]⁺. ¹H NMR (400 MHz, DMSO-d₆) δ 11.97 (s, 1H), 8.83 (s, 2H), 8.76 (s, 1H), 8.48 (s, 1H), 8.13 (s, 1H), 7.03 (s, 1H), 5.92 (s, 1H), 4.14 (s, 3H), 4.08 (s, 3H), 4.02 (s, 3H), 4.01-3.98 (m, 2H), 3.45-3.42 (m, 1H), 3.01-2.95 (m, 2H), 2.97 (s, 3H), 2.84-2.76 (m, 1H), 2.20 (d, J=10.5 Hz, 2H), 1.83-1.70 (m, 2H), 0.85 (d, J=5.6 Hz, 4H).

An analogous method was followed to obtain the following compounds.

| | Starting | |
|---|---|---|
| Compound | Material | Characterization |
| 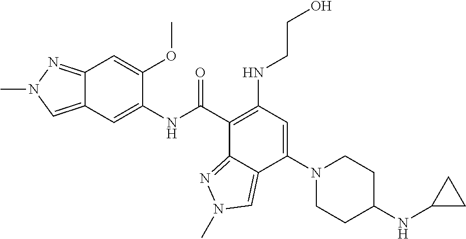 [SMILES: COc1cc2nn(C)cc2cc1NC(=O)c1c(NCCO)cc(N2CCC(NC3CC3)CC2)c2cn(C)nc12] | 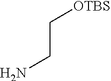 [SMILES: CC(C)(C)[Si](C)(C)OCCN] | Modification: Step 1 was ran in dioxane. The crude product was purified by Prep- HPLC (Condition 6, Gradient 8) to afford 4-[4-(cyclopropylamino)-1- piperidyl]-6-(2-hydroxyethylamino)-N- (6-methoxy-2-methyl-indazol-5-yl)-2- methyl-indazole-7-carboxamide (Compound 441, 12.4 mg, 23.3 μmol, 22% yield) as a solid. LCMS (ES, m/z): 533 [M + H]⁺. ¹H NMR (400 MHz, DMSO-d₆) δ 11.98 (s, 1H), 9.96 (t, J = 5.1 Hz, 1H), 8.77 (s, 1H), 8.39 (s, 1H), 8.12 (s, 1H), 7.01 (s, 1H), 5.90 (s, 1H), 4.88 (s, 1H), 4.13 (s, 3H), 4.08 (s, 3H), |
| Compound 441 | | 4.02 (s, 3H), 3.80 (d, J = 12.6 Hz, 2H), |
| | | 3.68 (t, J = 5.6 Hz, 2H), 3.36 (d, J = 5.5 |
| | | Hz, 2H), 2.97 (t, J = 11.5 Hz, 2H), 2.75 |
| | | (dt, J= 10.1, 5.6 Hz, 1H), 2.18-2.06 (m, |
| | | 1H), 2.00 (d, J = 12.4 Hz, 2H), 1.48 (q, |
| | | J= 12.1, 10.8 Hz, 2H), 0.40 (dt, J = 6.2, |
| | | 3.0 Hz, 2H), 0.28-0.20 (m, 2H). |
| 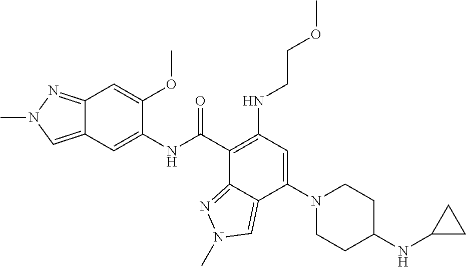 [SMILES: COCCNc1cc(N2CCC(NC3CC3)CC2)c2cn(C)nc2c1C(=O)Nc1cc2cn(C)nc2cc1OC] | 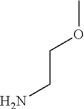 | Modification: Step 1 was ran in dioxane. The crude product was purified by Prep- HPLC (Condition 6, Gradient 4) to afford 4-[4-(cyclopropylamino)-1- piperidyl]-6-(2-methoxyethylamino)- N-(6-methoxy-2-methyl-indazol-5-yl)- 2-methyl-indazole-7-carboxamide (Compound 442, 29.8 mg, 54.5 umol, 39% yield) as a solid. LCMS (ES, m/z): 547 [M + H]⁺. ¹H NMR (400 MHz, DMSO-d₆) δ 11.99 (s, 1H), 9.93 (t, J = 5.2 Hz, 1H), 8.76 (s, 1H), 8.41 (s, 1H), 8.13 (s, 1H), 7.02 (s, 1H), 5.91 (s, 1H), 4.13 (s, 3H), 4.08 (s, 3H), 4.02 (s, 3H), 3.82 (d, J = 12.4 Hz, 2H), 3.62 (t, J = 5.3 Hz, 2H), 3.47 (q, J = 5.3 Hz, 2H), |
| Compound 442 | | 3.35 (s, 3H), 2.97 (t, J = 11.7 Hz, 2H), |
| | | 2.82 (s, 1H), 2.19 (s, 1H), 2.01 (d, J = |
| | | 12.3 Hz, 2H), 1.57-1.44 (m, 2H), 0.48- |
| | | 0.41 (m, 2H), 0.30 (s, 2H). |
| 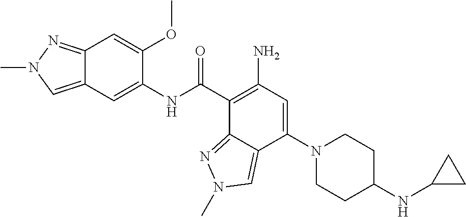 [SMILES: COc1cc2nn(C)cc2cc1NC(=O)c1c(N)cc(N2CCC(NC3CC3)CC2)c2cn(C)nc12] | 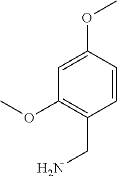 | Modification: Step 1 was ran in dioxane for 3 h at 80° C. Step 2 was ran for 2 h. The crude product was purified by Prep- HPLC (Condition 6, Gradient 5) to afford 6-amino-4-[4- (cyclopropylamino)-1-piperidyl]-N-(6- methoxy-2-methyl-indazol-5-yl)-2- methyl-indazole-7-carboxamide (Compound 450, 21.5 mg, 44 μmol, 41% yield) as a solid. LCMS (ES, m/z): 489 [M + H]⁺. ¹H NMR (400 MHz, DMSO-d₆) δ 11.86 (s, 1H), 8.76 (s, 1H), 8.32 (s, 1H), 8.12 (s, 1H), 7.01 (s, 1H), 5.90 (s, 1H), 4.09 (s, |
| Compound 450 | | 3H), 4.05 (s, 3H), 4.01 (s, 3H), 3.70 (d, |
| | | J = 12.6 Hz, 2H), 2.88 (t, J = 11.7 Hz, |
| | | 2H), 2.75-2.72 (m, 1H), 2.26-2.21 (m, |
| | | 1H), 2.13 (dq, J = 6.6, 3.4 Hz, 1H), 1.98 |
| | | (d, J = 12.1 Hz, 2H), 1.46 (q, J = 10.1 |
| | | Hz, 2H), 0.44-0.36 (m, 2H), 0.24 (q, J = |
| | | 3.3, 2.7 Hz, 2H). |

### Example 147: Synthesis of C130

### Synthesis of C126

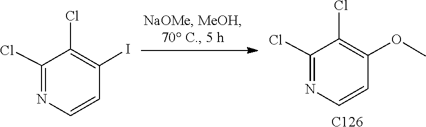

A solution of 2,3-dichloro-4-iodo-pyridine (10 g, 36.51 mmol) in MeOH (100 mL) was treated with NaOMe (1.97 g, 36.51 mmol), and the reaction was stirred for 5 h at 70° C. The resulting mixture was concentrated under reduced pressure and extracted with EtOAc (2×100 mL). The combined organic layers were washed with brine (100 mL), dried over anhydrous Na₂SO₄, and filtered. The filtrate was concentrated under reduced pressure and purified by silica gel column chromatography (33% EtOAc in PE) to afford 2,3-dichloro-4-methoxy-pyridine (4.4 g, 24.7 mmol, 68% yield) as a solid. LCMS (ES, m/z): 178 [M+H]⁺.

### Synthesis of C127

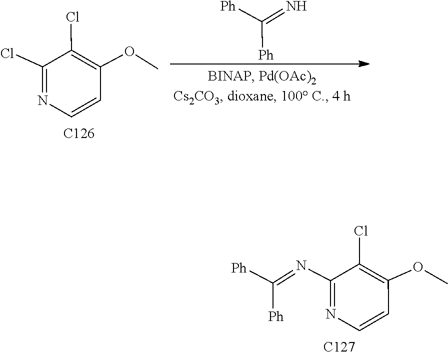

To a stirred mixture of diphenylmethanimine (4.70 g, 25.95 mmol, 4.36 mL) and 2,3-dichloro-4-methoxy-pyridine (4.2 g, 23.59 mmol) in dioxane (50 mL) were added Cs₂CO₃ (23.06 g, 70.78 mmol), BINAP (2.94 g, 4.72 mmol) and Pd(OAc)₂ (529.7 mg, 2.36 mmol, 241.9 μL), and the reaction was stirred for 4 h at 100° C. under N₂. The resulting mixture was allowed to cool to room temperature and purified by silica gel column chromatography (33% EtOAc in PE) to afford N-(3-chloro-4-methoxy-2-pyridyl)-1,1-diphenyl-methanimine (6 g, 18.6 mmol, 79% yield) as a solid. LCMS (ES, m/z): 323 [M+H]⁺.

### Synthesis of C128

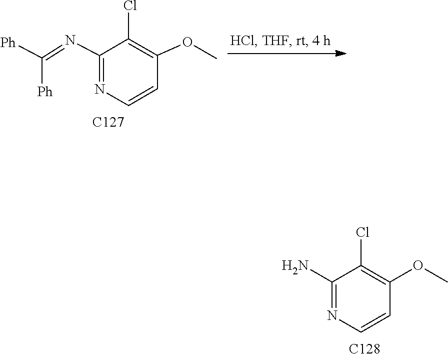

A solution of N-(3-chloro-4-methoxy-2-pyridyl)-1,1-diphenyl-methanimine (5.8 g, 17.97 mmol) in THF (60 mL) was treated with HCl (conc.) (60 mL) at 0° C. The resulting mixture was stirred for 4 h at room temperature under N₂. The precipitated solids were collected by filtration and washed with water (2×50 mL). The resulting solid was dried under infrared light to afford 3-chloro-4-methoxy-pyridin-2-amine hydrochloride (2.1 g, 10.8 mmol, 60% yield) as a solid. LCMS (ES, m/z): 159 [M+H]⁺.

### Synthesis of C129

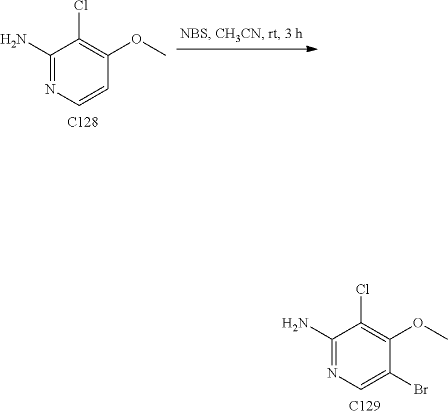

A solution of 3-chloro-4-methoxy-pyridin-2-amine hydrochloride (2 g, 12.61 mmol) in MeCN (20 mL) was treated with NBS (2.47 g, 13.87 mmol) under N₂, and the reaction was stirred for 3 h at room temperature under N₂. The resulting mixture was diluted with water (20 mL) and extracted with EtOAc (3×50 mL). The combined organic layers were washed with brine (40 mL), dried over anhydrous Na₂SO₄, and filtered. The filtrate was concentrated under reduced pressure, to afford 5-bromo-3-chloro-4-methoxy-pyridin-2-amine (2.9 g, 12.2 mmol, 97% yield) as a solid. LCMS (ES, m/z): 237 [M+H]⁺.

### Synthesis of C130

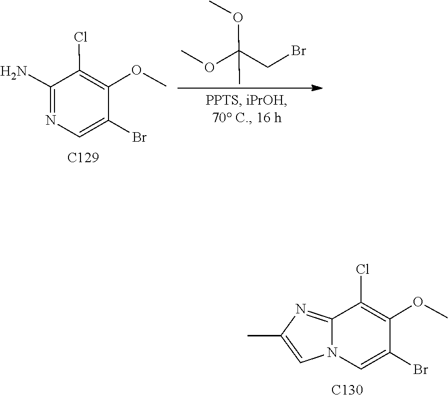

To a stirred mixture of 1-bromo-2,2-dimethoxy-propane (1.16 g, 6.32 mmol) and 5-bromo-3-chloro-4-methoxy-pyridin-2-amine (1 g, 4.21 mmol) in iPrOH (10 mL) were added PPTS (105 mg, 421.1 μmol), and the reaction was stirred for 16 h at 70° C. The reaction was quenched by the addition of water (20 mL) at room temperature and extracted with EtOAc (3×30 mL). The combined organic layers were washed with water (2×40 mL) and brine (40 mL), dried over anhydrous Na₂SO₄, and filtered. The filtrate was concentrated under reduced pressure and purified by silica gel column chromatography (50% EtOAc in PE) to afford 6-bromo-8-chloro-7-methoxy-2-methyl-imidazo[1,2-a]pyridine (260 mg, 943.6 μmol, 22% yield) as a solid. LCMS (ES, m/z): 275 [M+H]⁺.

### Example 148: Synthesis of C133

### Synthesis of C131

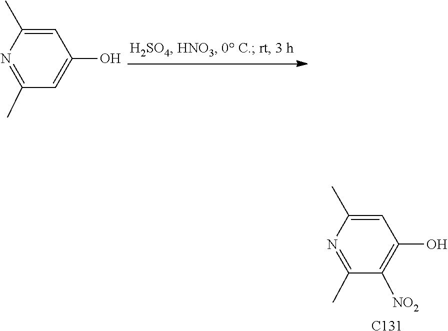

To a stirred mixture of 2,6-dimethylpyridin-4-ol (12.0 g, 97.44 mmol) in HNO₃ (44 mL) was added H₂SO₄ (64 mL) dropwise at 0° C. The resulting mixture was stirred for 16 h at rt. The resulting mixture was diluted with ice-water (500 ml). The mixture was acidified to pH 7 with K₂CO₃. The resulting mixture was filtered, the filter cake was washed with H₂O (50 ml) and dried to afford 2,6-dimethyl-3-nitro-pyridin-4-ol (8 g, 47.6 mmol, 49% yield) as a solid. LCMS (ES, m/z): 169 [M+H]⁺.

### Synthesis of C132

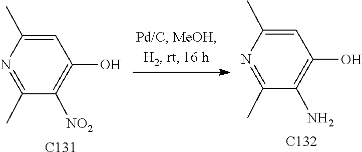

To a solution of 2,6-dimethyl-3-nitro-pyridin-4-ol (6 g, 35.68 mmol) in MeOH (150 mL) was added Pd/C (0.7 g, 10%) under N₂ in a 500 mL round-bottom flask. The mixture was hydrogenated at room temperature for 16 h under hydrogen atmosphere using a hydrogen balloon, filtered through a Celite pad and concentrated under reduced pressure to afford 3-amino-2,6-dimethyl-pyridin-4-ol (4 g, 29 mmol, 81% yield) as a solid. LCMS (ES, m/z): 139 [M+H]⁺.

### Synthesis of C133

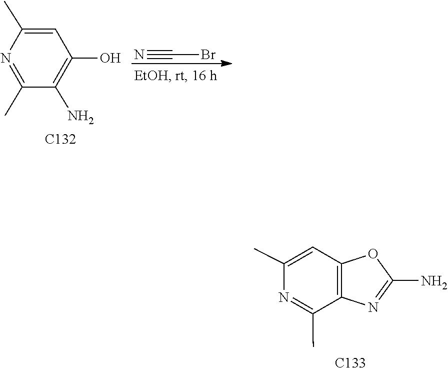

To a stirred mixture of 3-amino-2,6-dimethyl-pyridin-4-ol (1.2 g, 8.69 mmol) in EtOH (12 mL) was added carbononitridic bromide (1.20 g, 11.29 mmol), and the reaction was stirred for 16 h at room temperature. The precipitated solids were collected by filtration and washed with EtOH (10 mL), to afford 4,6-dimethyloxazolo[4,5-c]pyridin-2-amine (1.3 g, 7.97 mmol, 92% yield) as a solid. LCMS (ES, m/z): 164 [M+H]⁺.

### Example 149: Synthesis of C140

### Synthesis of C135

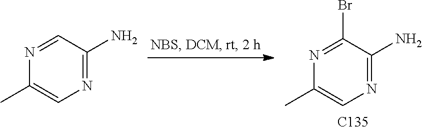

To a solution of 5-methylpyrazin-2-amine (24 g, 219.9 mmol) in DCM (250 mL) was added NBS (39.14 g, 219.9 mmol), and the reaction was stirred for 2 h at rt. The reaction was diluted with H₂O (400 mL) and extracted with DCM (2×300 mL). The combined organic layers were washed with water (2×500 mL), dried over anhydrous Na₂SO₄, and filtered. The filtrate was concentrated under reduced pressure and purified by silica gel column chromatography (33% EtOAc in PE) to afford 3-bromo-5-methyl-pyrazin-2-amine (23 g, 122.3 mmol, 56% yield) as a solid. LCMS (ES, m/z): 188 [M+H]⁺.

### Synthesis of C136

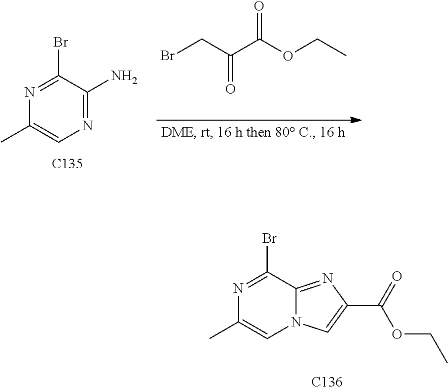

To a solution of 3-bromo-5-methyl-pyrazin-2-amine (22.5 g, 119.7 mmol) in DME (450 mL) was added ethyl 3-bromo-2-oxo-propanoate (28 g, 143.6 mmol). The mixture was stirred for 16 h at rt and then stirred for 16 h at 80° C. The resulting mixture was neutralized to PH=7 with NaHCO₃ (aq.) and extracted with EtOAc (2×300 mL). The combined organic layers were washed with water (2×500 mL), dried over anhydrous Na₂SO₄, and filtered. The filtrate was concentrated under reduced pressure and purified by silica gel column chromatography (50% EtOAc in PE) to afford ethyl 8-bromo-6-methyl-imidazo[1,2-a]pyrazine-2-carboxylate (14.5 g, 51.04 mmol, 43% yield) as a solid. LCMS (ES, m/z): 284 [M+H]⁺.

### Synthesis of C137

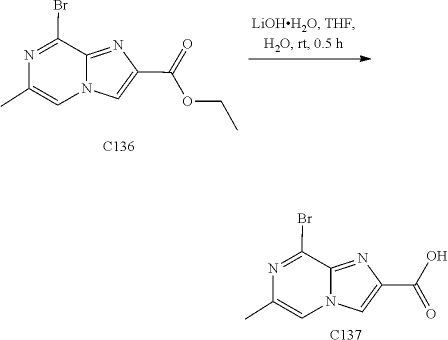 [SMILES: CCOC(=O)c1cn2cc(C)nc(Br)c2n1 ; "C136" ; Cc1cn2cc(C(=O)O)nc2c(Br)n1 ; "C137"]

To a stirred solution of ethyl 8-bromo-6-methyl-imidazo[1,2-a]pyrazine-2-carboxylate (2 g, 7.04 mmol) in THF (15 mL) and H₂O (15 mL) was added LiOH·H₂O (1.48 g, 35.2 mmol) in portions at rt. The resulting mixture was stirred for 30 min at rt. The resulting mixture was diluted with water (100 mL). The mixture was acidified to pH 6 with HCl (2N) at 0° C. The precipitated solids were collected by filtration and washed with H₂O (20 mL). The solid was dried to afford 8-bromo-6-methyl-imidazo[1,2-a]pyrazine-2-carboxylic acid (1.4 g, 5.47 mmol, 78% yield) as a solid. LCMS (ES, m/z): 256 [M+H]⁺.

### Synthesis of C138

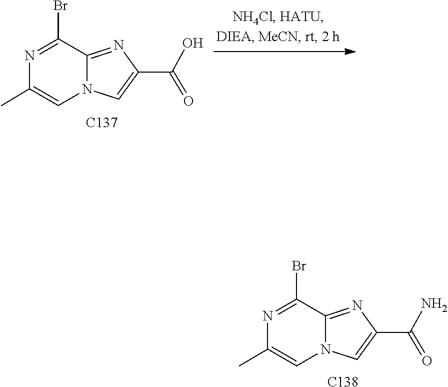

To a stirred mixture of 8-bromo-6-methyl-imidazo[1,2-a]pyrazine-2-carboxylic acid (7.0 g, 27.34 mmol) in DCM (100 mL) was added DIEA (14.1 g, 109.4 mmol) and HATU (17.6 g, 46.47 mmol) and NH₄Cl (2.90 g, 54.68 mmol), and the reaction was stirred for 3 h at room temperature. The resulting mixture was diluted with water (100 mL). The precipitated solids were collected by filtration and washed with DCM (20 mL). The residue was purified by trituration with H₂O (500 mL). The precipitated solids were collected by filtration and washed with H₂O (20 mL) to afford 8-bromo-6-methyl-imidazo[1,2-a]pyrazine-2-carboxamide (5 g, 19.6 mmol, 72% yield) as a solid. LCMS (ES, m/z): 255 [M+H]⁺.

### Synthesis of C139

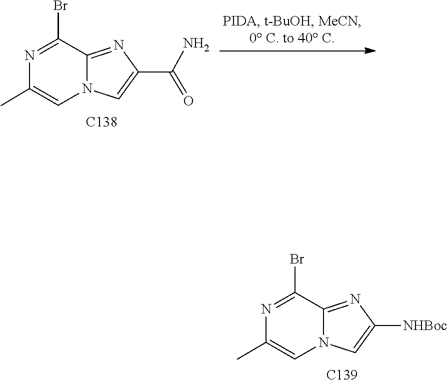 [SMILES: Cc1cn2cc(C(N)=O)nc2c(Br)n1 ; "C138" ; Cc1cn2cc(NC(=O)OC(C)(C)C)nc2c(Br)n1 ; "C139"]

To a stirred mixture of 8-bromo-6-methyl-imidazo[1,2-a]pyrazine-2-carboxamide (5 g, 19.6 mmol) in MeCN (100 mL) and t-BuOH (50 mL) was added [acetoxy (phenyl)-iodanyl] acetate (18.94 g, 58.81 mmol) in portions at 0° C., and the reaction was stirred for 2 h at 40° C. The residue was purified by reversed-phase flash chromatography (Condition 3, Gradient 29). The liquid was concentrated under reduced pressure. The resulting mixture was extracted with EtOAc (300 mL), dried over anhydrous Na₂SO₄, and filtered. The filtrate was concentrated under reduced pressure to afford tert-butyl N-(8-bromo-6-methyl-imidazo[1,2-a]pyrazin-2-yl)carbamate (3.8 g, 11.6 mmol, 59% yield) as a solid. LCMS (ES, m/z): 327 [M+H]⁺.

### Synthesis of C140

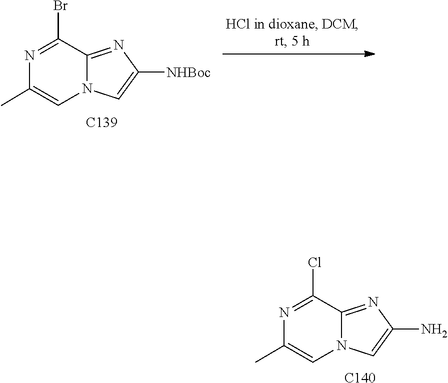 [SMILES: Cc1cn2cc(NC(=O)OC(C)(C)C)nc2c(Br)n1 ; "C139" ; Cc1cn2cc(N)nc2c(Cl)n1 ; "C140"]

To a solution of tert-butyl N-(8-bromo-6-methyl-imidazo[1,2-a]pyrazin-2-yl)carbamate (200 mg, 611.3 μmol) in DCM (4 mL) was added HCl (4.0 M in 1,4-dioxane, 1 mL). The reaction was stirred for 5 h at rt. The resulting mixture was concentrated under reduced pressure to afford 8-chloro-6-methyl-imidazo[1,2-a]pyrazin-2-amine hydrochloride (100 mg, 547.6 μmol, 90% yield) as a solid. LCMS (ES, m/z): 183 [M+H]⁺.

An analogous method was followed to obtain the following compounds.

| | Starting | |
|---|---|---|
| Compound | Material | Characterization |
| 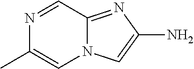 | 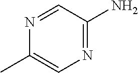 | Modification: Step 1 was proceeded to run in EtOH at 85º C. for 2 h, after the initial 16 h. Step 2 was ran for 3 h. Step 5 was run at room temperature for 16 h. The resulting mixture was concentrated under reduced pressure to afford 6-methylimidazo[1,2-a]pyrazin-2- |
| C156 | | amine hydrochloride (C156, 180 mg, 974.9 μmol, 62% |
| | | yield) as a solid. LCMS (ES, m/z): 149 [M + H]⁺. |

### Example 150: Synthesis of Compound 390

### Synthesis of B125

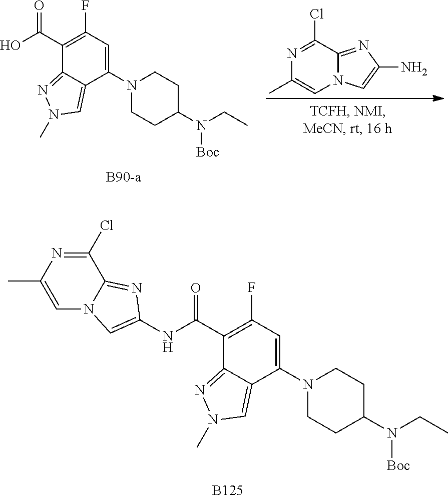

To a stirred mixture of 4-[4-[tert-butoxycarbonyl(ethyl)amino]-1-piperidyl]-6-fluoro-2-methyl-indazole-7-carboxylic acid (200 mg, 475.7 μmol), 8-chloro-6-methylimidazo[1,2-a]pyrazin-2-amine hydrochloride (114 mg, 523.2 μmol) and NMI (198 mg, 2.38 mmol) in MeCN (4 mL) was added TCFH (187 mg, 665.9 μmol), and the reaction was stirred for 16 h at room temperature. The resulting mixture was quenched with water (10 mL) and extracted with EtOAc (2×10 mL). The combined organic layers were washed with brine (10 mL), dried over anhydrous Na₂SO₄, and filtered. The filtrate was concentrated under reduced pressure and purified by silica gel column chromatography (67% EtOAc in PE) to afford tert-butyl N-[1-[7-[(8-chloro-6-methyl-imidazo[1,2-a]pyrazin-2-yl)carbamoyl]-6-fluoro-2-methyl-indazol-4-yl]-4-piperidyl]-N-ethyl-carbamate (130 mg, 222.2 μmol, 47% yield) as a solid. LCMS (ES, m/z): 585 [M+H]⁺.

### Synthesis of B126

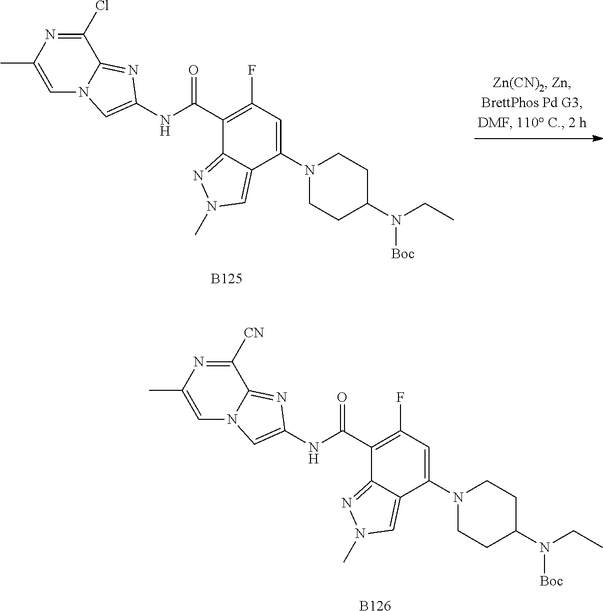

To a solution of tert-butyl N-[1-[7-[(8-chloro-6-methyl-imidazo[1,2-a]pyrazin-2-yl)carbamoyl]-6-fluoro-2-methyl-indazol-4-yl]-4-piperidyl]-N-ethyl-carbamate (80 mg, 136.7 μmol) and Zn(CN)₂ (21 mg, 177.8 μmol) in DMF (1.5 mL) were added BrettPhos Pd G3 (12.4 mg, 13.7 μmol), and the reaction was stirred for 2 h at 110° C. under N₂. The resulting mixture was concentrated under reduced pressure and purified by silica gel column chromatography (67% EtOAc in PE) to afford tert-butyl N-[1-[7-[(8-cyano-6-methyl-imidazo[1,2-a]pyrazin-2-yl)carbamoyl]-6-fluoro-2-methyl-indazol-4-yl]-4-piperidyl]-N-ethyl-carbamate (50 mg, 86.9 μmol, 64% yield) as a solid. LCMS (ES, m/z): 576 [M+H]⁺.

### Synthesis of Compound 390

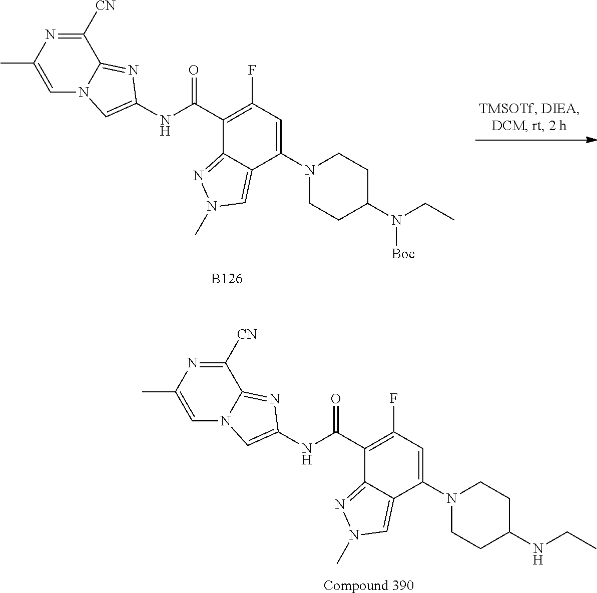

To a stirred mixture of tert-butyl N-[1-[7-[(8-cyano-6-methyl-imidazo[1,2-a]pyrazin-2-yl)carbamoyl]-6-fluoro-2-methyl-indazol-4-yl]-4-piperidyl]-N-ethyl-carbamate (80 mg, 139 μmol) and DIEA (45 mg, 347.4 μmol) in DCM (2 mL) was added TMSOTf (77 mg, 347.4 μmol) dropwise, and the reaction was stirred for 2 h at room temperature. The resulting mixture was purified by reversed-phase flash chromatography (Condition 3, Gradient 11) to afford N-(8-cyano-6-methyl-imidazo[1,2-a]pyrazin-2-yl)-4-[4-(ethylamino)-1-piperidyl]-6-fluoro-2-methyl-indazole-7-carboxamide (10 mg, 21 μmol, 15% yield) as a solid. LCMS (ES, m/z): 476 [M+H]⁺. ¹H NMR (300 MHz, DMSO-d₆) δ 11.84 (s, 1H), 8.87-8.79 (m, 2H), 8.58 (s, 1H), 6.26 (d, J=16.2 Hz, 1H), 4.26 (s, 3H), 3.95 (d, J=13.1 Hz, 2H), 3.16 (t, J=11.8 Hz, 2H), 2.72 (s, 1H), 2.60 (q, J=7.1 Hz, 2H), 2.49 (s, 3H), 1.95 (d, J=12.9 Hz, 2H), 1.42 (t, J=10.5 Hz, 2H), 1.04 (t, J=7.1 Hz, 3H).

An analogous method for this step was followed to obtain the following compounds.

| | | |
|---|---|---|
| Compound | Starting Material | Characterization |

| | | |
|---|---|---|
| 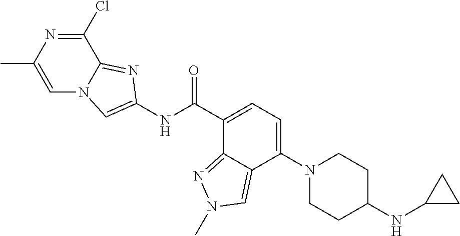 [SMILES: Cc1cn2cc(NC(=O)c3ccc(N4CCC(NC5CC5)CC4)c4cn(C)nc34)nc2c(Cl)n1] | 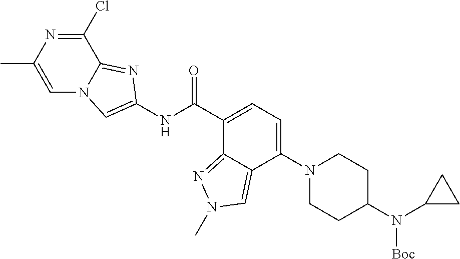 [SMILES: Cc1cn2cc(NC(=O)c3ccc(N4CCC(N(C(=O)OC(C)(C)C)C5CC5)CC4)c4cn(C)nc34)nc2c(Cl)n1] | The residue was purified by reversed-phase flash chromatography (Condition 3, Gradient 33) to afford N-(8- chloro-6-methyl- imidazo[1,2-a]-pyrazin- 2-yl)-4-[4- (cyclopropylamino)-1- piperidyl]-2-methyl- indazole-7-carboxamide (Compound 479, 10 mg, 20.9 μmol). LCMS (ES, m/z): 479 [M + H]⁺. ¹H NMR (400 MHz, DMSO-d₆) δ 11.55 (s, |
| Compound 479 | | 1H), 8.81 (s, 1H), 8.52 |
| | | (d, J = 8.5 Hz, 2H), 8.03 |
| | | (d, J = 8.1 Hz, 1H), 6.51 |
| | | (d, J = 8.3 Hz, 1H), 4.31 |
| | | (s, 3H), 3.91 (d, J = 12.8 |
| | | Hz, 2H), 3.09 (t, J= 11.4 |
| | | Hz, 2H), 2.80 (t, J = 7.2 |
| | | Hz, 1H), 2.40 (s, 3H), |
| | | 2.13 (tt, J = 6.7, 3.7 Hz, |
| | | 1H), 2.01 (d, J = 12.7 |
| | | Hz, 2H), 1.48 (q, J = |
| | | 10.1 Hz, 2H), 0.41 (td, J = |
| | | 6.3, 4.0 Hz, 2H), 0.28- |
| | | 0.20 (m, 2H). |

### Example 151: Synthesis of Compound 391

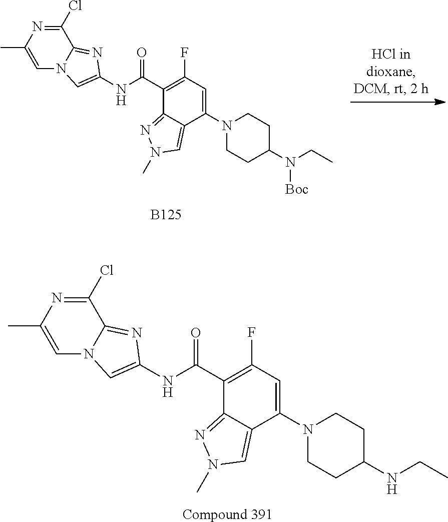

To a solution of tert-butyl N-[1-[7-[(8-chloro-6-methyl-imidazo[1,2-a]pyrazin-2-yl)-carbamoyl]-6-fluoro-2-methyl-indazol-4-yl]-4-piperidyl]-N-ethyl-carbamate (20 mg, 34.18 μmol) in DCM (0.3 mL) was added HCl (4.0 M in 1,4-dioxane, 0.1 mL). The reaction was stirred for 2 h at rt. The resulting mixture was concentrated under reduced pressure and adjusted to pH 8 with saturated NaHCO₃ (aq.). The resulting mixture was purified by reversed-phase flash chromatography (Condition 3, Gradient 27) to afford N-(8-chloro-6-methyl-imidazo[1,2-a]-pyrazin-2-yl)-4-[4-(ethylamino)-1-piperidyl]-6-fluoro-2-methyl-indazole-7-carboxamide (Compound 391, 7 mg, 14.4 μmol, 42% yield) as a solid. LCMS (ES, m/z): 485 [M+H]⁺. ¹H NMR (300 MHz, DMSO-d₆) δ 11.66 (s, 1H), 8.81 (s, 1H), 8.49 (s, 1H), 8.44 (s, 1H), 6.23 (d, J=16.1 Hz, 1H), 4.23 (s, 3H), 3.91 (d, J=13.0 Hz, 2H), 3.12 (t, J=11.7 Hz, 2H), 2.68 (dd, J=10.9, 6.5 Hz, 1H), 2.57 (t, J=7.1 Hz, 2H), 2.35 (s, 3H), 1.93 (d, J=12.4 Hz, 2H), 1.69-1.52 (m, 1H), 1.38 (q, J=9.9 Hz, 2H), 1.01 (t, J=7.1 Hz, 3H).

### Example 152: Synthesis of C144

### Synthesis of C141

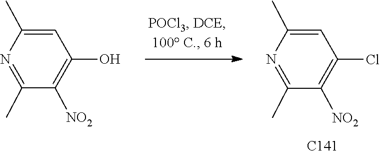

To a solution of 2,6-dimethyl-3-nitro-pyridin-4-ol (9.8 g, 58.28 mmol) in DCE (100 mL) was added POCl₃ (44.68 g, 291.4 mmol, 27 mL), and the reaction was stirred for 6 h at 100° C. The resulting mixture was concentrated under reduced pressure, acidified to pH 8 with saturated NaHCO₃ (aq.), and extracted with DCM (2×100 mL). The combined organic layers were washed with water (200 mL) and dried over anhydrous Na₂SO₄, and filtered. The filtrate was concentrated under reduced pressure and purified by silica gel column chromatography (60% DCM in PE) to afford 4-chloro-2,6-dimethyl-3-nitro-pyridine (9.8 g, 52.5 mmol, 90% yield) as a solid. LCMS (ES, m/z): 187 [M+H]⁺.

### Synthesis of C142

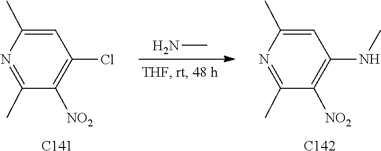

To a solution of 4-chloro-2,6-dimethyl-3-nitro-pyridine (4.9 g, 26.26 mmol) in THF (50 mL) was added methanamine (2 M in THF, 26 mL), and the reaction was stirred for 48 h at rt. The reaction was diluted with water (300 mL) and extracted with EtOAc (2×300 mL). The combined organic layers were washed with water (2×400 mL), dried over anhydrous Na₂SO₄, and filtered. The filtrate was concentrated under reduced pressure, to afford N,2,6-trimethyl-3-nitro-pyridin-4-amine (4.7 g, 25.94 mmol, 99% yield) as a solid. LCMS (ES, m/z): 182 [M+H]⁺.

### Synthesis of C143

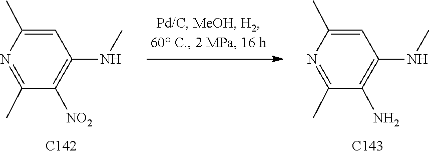

To a solution of N,2,6-trimethyl-3-nitro-pyridin-4-amine (4.7 g, 25.94 mmol) in MeOH (150 mL) was added Pd/C (10% on Carbon (wetted with ca. 55% Water), 1.10 g, 10.38 mmol) in a pressure tank. The mixture was pressurized to 20 atm with H₂ for 16 h at 60° C. The reaction mixture was cooled to room temperature and filtered. The resulting mixture was concentrated under reduced pressure to afford N4,2,6-trimethylpyridine-3,4-diamine (3.65 g, 24.1 mmol, 93% yield) as an oil. LCMS (ES, m/z): 152 [M+H]⁺.

### Synthesis of C144

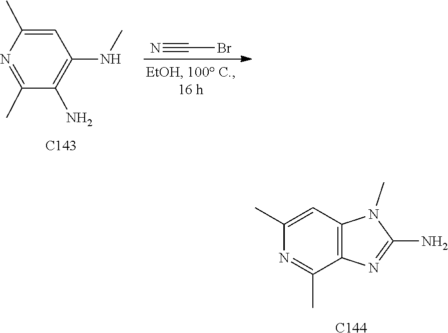

To a solution of N4,2,6-trimethylpyridine-3,4-diamine (1.5 g, 9.92 mmol) in EtOH (20 mL) was added cyanic bromide (1.37 g, 12.9 mmol), and the reaction was stirred for 16 h at 100° C. The resulting mixture was concentrated under reduced pressure and purified by silica gel column chromatography (67% EtOAc in DCM) to afford 1,4,6-trimethylimidazo[4,5-c]pyridin-2-amine (1.29 g, 7.32 mmol, 74% yield) as a solid. LCMS (ES, m/z): 177 [M+H]⁺.

### Example 153: Synthesis of Compound 651

### Synthesis of B127

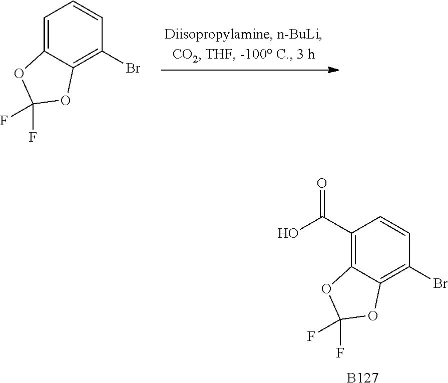

Diisopropylamine (1.18 mL, 8.4 mmol) and 4-bromo-2,2-difluoro-1,3-benzodioxole (2.0 g, 8.4 mmol) were added to a cooled (−100° C.) solution of n-butyllithium (1.6 M, in hexane, 5.3 mL, 8.4 mmol) in THF (15 mL), and the reaction was stirred for 2 h. The resulting mixture was poured onto freshly crushed dry-ice (30 g). The resulting mixture was allowed to cool to room temperature, water was added, and the mixture was washed with DCM (10 mL). The aqueous phase was acidified with 2 M HCl, extracted with EtOAc, dried over anhydrous Na₂SO₄, filtered, and concentrated under reduced pressure to afford 7-bromo-2,2-difluoro-1,3-benzodioxole-4-carboxylic acid (B127, 1.5 g, 63% yield) as a solid. LCMS (ES, m/z): 279 [M+H]⁻.

### Synthesis of B128

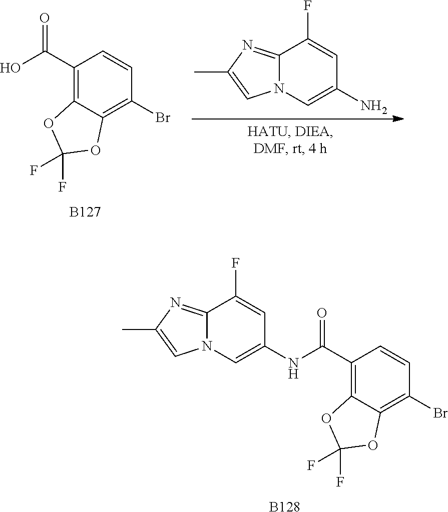 [SMILES: "B127" ; "B128" ; O=C(O)c1ccc(Br)c2c1OC(F)(F)O2 ; Cc1cn2cc(NC(=O)c3ccc(Br)c4c3OC(F)(F)O4)cc(F)c2n1 ; Cc1cn2cc(N)cc(F)c2n1]

To a stirred solution of 7-bromo-2,2-difluoro-1,3-benzodioxole-4-carboxylic acid (200 mg, 0.71 mmol, 1 eq) and DIEA (460 mg, 3.56 mmol, 5 eq) in DMF (4 mL) were added HATU (325 mg, 0.85 mmol, 1.2 eq) and 8-fluoro-2-methylimidazo[1,2-a]pyridin-6-amine (130 mg, 0.78 mmol, 1.1 eq), and the reaction was stirred for 4 h at room temperature. The resulting mixture was quenched by the addition of water (20 mL) at room temperature. The precipitated solids were collected by filtration and washed with water (2×5 mL), to afford 7-bromo-2,2-difluoro-N-(8-fluoro-2-methylimidazo[1,2-a]pyridin-6-yl)-1,3-benzodioxole-4-carboxamide (220 mg, 72% yield) as a solid. LCMS (ES, m/z): 428 [M+H]⁺.

### Synthesis of B129

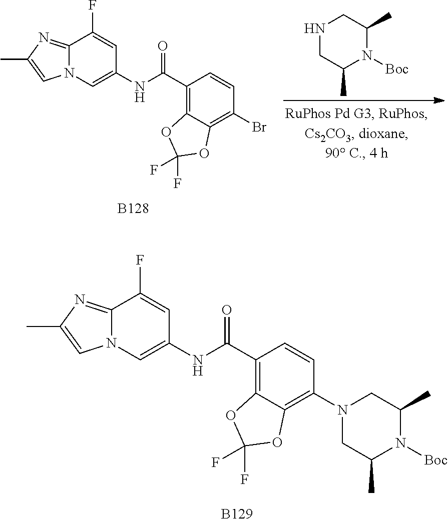 [SMILES: "B128" ; "B129" ; Cc1cn2cc(NC(=O)c3ccc(Br)c4c3OC(F)(F)O4)cc(F)c2n1 ; Cc1cn2cc(NC(=O)c3ccc(N4C[C@@H](C)N(C(=O)OC(C)(C)C)[C@@H](C)C4)c4c3OC(F)(F)O4)cc(F)c2n1 ; C[C@@H]1CNC[C@H](C)N1C(=O)OC(C)(C)C]

To a stirred solution of 7-bromo-2,2-difluoro-N-(8-fluoro-2-methylimidazo[1,2-a]pyridin-6-yl)-1,3-benzodioxole-4-carboxamide (220 mg, 0.51 mmol, 1 eq) and tert-butyl(2R,6S)-2,6-dimethylpiperazine-1-carboxylate (143 mg, 0.67 mmol, 1.3 eq) in dioxane (5 mL) were added Cs₂CO₃ (335 mg, 1.03 mmol, 2 eq), RuPhos (48 mg, 0.10 mmol, 0.2 eq) and RuPhos Pd G3 (43 mg, 0.05 mmol, 0.1 eq) at room temperature under N₂. The resulting mixture was stirred for 4 h at 90° C. under N₂. The resulting mixture was concentrated under reduced pressure and purified by reversed-phase flash chromatography (Condition 1, Gradient 7) to afford tert-butyl(2R,6S)-4-[2,2-difluoro-7-((8-fluoro-2-methylimidazo[1,2-a]pyridin-6-yl)carbamoyl)-1,3-benzodioxol-4-yl]-2,6-dimethylpiperazine-1-carboxylate (120 mg, 42% yield) as a solid. LCMS (ES, m/z): 562 [M+H]⁺.

### Synthesis of Compound 651

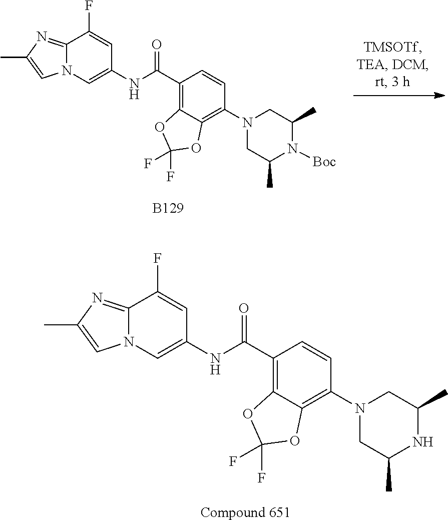

To a stirred solution of tert-butyl(2R,6S)-4-[2,2-difluoro-7-((8-fluoro-2-methylimidazo[1,2-a]pyridin-6-yl)carbamoyl)-1,3-benzodioxol-4-yl]-2,6-dimethylpiperazine-1-carboxylate (120 mg, 0.214 mmol, 1 eq) and TEA (109 mg, 1.07 mmol, 5 eq) in DCM (3 mL) was added TMSOTf (143 mg, 0.64 mmol, 3 eq), and the reaction was stirred for 3 h at room temperature. The resulting mixture was concentrated under reduced pressure and purified by reversed-phase flash chromatography (Condition 3, Gradient 7) to afford 7-[(3R,5S)-3,5-dimethylpiperazin-1-yl]-2,2-difluoro-N-(8-fluoro-2-methylimidazo[1,2-a]-pyridin-6-yl)-1,3-benzodioxole-4-carboxamide (Compound 651, 30 mg, 30% yield) as a solid. LCMS (ES, m/z): 462 [M+H]⁺. ¹H NMR (300 MHz, DMSO-d₆) δ 10.17 (s, 1H), 9.04 (d, J=1.6 Hz, 1H), 7.90 (d, J=3.1 Hz, 1H), 7.57 (d, J=9.0 Hz, 1H), 7.27 (dd, J=12.7, 1.6 Hz, 1H), 6.89 (d, J=9.1 Hz, 1H), 3.64 (d, J=10.8 Hz, 2H), 2.85 (d, J=7.1 Hz, 2H), 2.41 (t, J=11.2 Hz, 3H), 2.37-2.31 (m, 3H), 1.03 (d, J=6.3 Hz, 6H).

### Example 154: Synthesis of B130

### Synthesis of B130

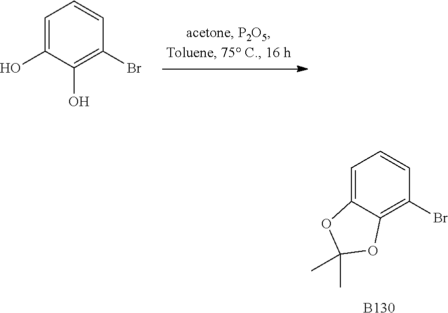

To a stirred solution of 3-bromobenzene-1,2-diol (2 g, 10.58 mmol) and acetone (1.23 g, 21.16 mmol) in Toluene (20 mL) was added Phosphorus (V) pentoxide (1.65 g, 11.64 mmol) at 75° C. The resulting mixture was stirred for 16 h at 75° C. The mixture was allowed to cool down to room temperature. The reaction was under reduced pressure and purified by silica gel column chromatography (5% THF in PE) to afford 4-bromo-2,2-dimethyl-1,3-benzodioxole (400 mg, 1.75 mmol, 17% yield) as an oil. LCMS (ES, m/z): 229 [M+H].

### Example 155: Synthesis of C147

### Synthesis of C145

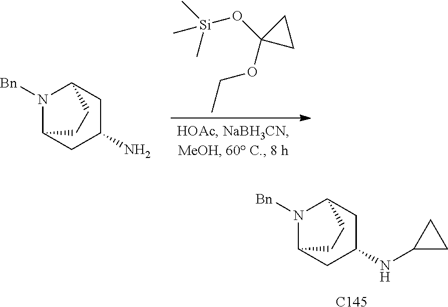 [SMILES: N[C@H]1C[C@H]2CC[C@@H](C1)N2Cc1ccccc1 ; CCOC1(O[Si](C)(C)C)CC1 ; c1ccc(CN2[C@@H]3CC[C@H]2C[C@@H](NC2CC2)C3)cc1 ; "C145"]

A solution of (1R,5S)-8-benzyl-8-azabicyclo[3.2.1]octan-3-amine (1 g, 4.62 mmol) and HOAc (1.11 g, 18.49 mmol) in MeOH (30 mL) as treated with (1-ethoxycyclopropoxy)-trimethyl-silane (1.61 g, 9.25 mmol, 1.86 mL) followed by the addition of NaBH₃CN (871 mg, 13.87 mmol), and the reaction was stirred for 8 h at 60° C. The resulting mixture was diluted with water (20 mL) and extracted with EtOAc (3×40 mL). The combined organic layers were washed with brine (30 mL), dried over anhydrous Na₂SO₄, and filtered. The filtrate was concentrated under reduced pressure and purified by silica gel column chromatography (9% MeOH in DCM) to afford (1R,5S)-8-benzyl-N-cyclopropyl-8-azabicyclo[3.2.1]octan-3-amine (1.1 g, 4.3 mmol, 93% yield) as an oil. LCMS (ES, m/z): 257 [M+H]⁺.

### Synthesis of C146

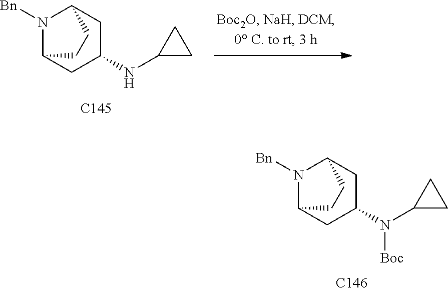 [SMILES: c1ccc(CN2[C@@H]3CC[C@H]2C[C@@H](NC2CC2)C3)cc1 ; "C145" ; CC(C)(C)OC(=O)N(C1CC1)[C@H]1C[C@H]2CC[C@@H](C1)N2Cc1ccccc1 ; "C146"]

A solution of (1R,5S)-8-benzyl-N-cyclopropyl-8-azabicyclo[3.2.1]octan-3-amine (1 g, 3.90 mmol) in DCM (20 mL) was treated with NaH (60% dispersion in oil) (187 mg, 7.80 mmol) for 30 min at 0° C. under N₂ followed by the addition of Boc₂O (2.55 g, 11.7 mmol) in portions at 0° C. The resulting mixture was stirred for 3 h at room temperature. The reaction was quenched with sat. NH₄Cl (aq.) at 0° C. The resulting mixture was extracted with DCM (2×50 mL). The combined organic layers were washed with water (2×50 mL), dried over anhydrous Na₂SO₄, and filtered. The filtrate was concentrated under reduced pressure and purified by silica gel column chromatography (20% EtOAc in PE) to afford tert-butyl N-[(1R,5S)-8-benzyl-8-azabicyclo[3.2.1]octan-3-yl]-N-cyclopropyl-carbamate (870 mg, 2.44 mmol, 63% yield) as an oil. LCMS (ES, m/z): 357 [M+H]⁺.

### Synthesis of C147

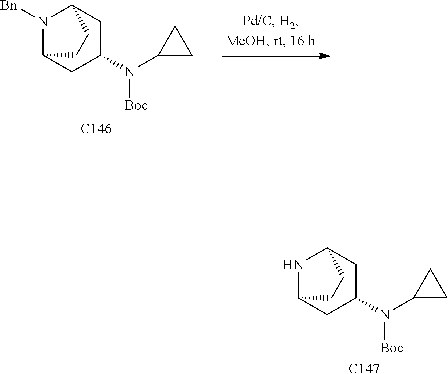 [SMILES: CC(C)(C)OC(=O)N(C1CC1)[C@H]1C[C@H]2CC[C@@H](C1)N2Cc1ccccc1 ; "C146" ; CC(C)(C)OC(=O)N(C1CC1)[C@H]1C[C@H]2CC[C@@H](C1)N2 ; "C147"]

To a solution of tert-butyl N-[(1R,5S)-8-benzyl-8-azabicyclo[3.2.1]octan-3-yl]-N-cyclopropyl-carbamate (870 mg, 2.44 mmol) in MeOH (18 mL) was added Pd/C (90 mg, 10%, wet) under N₂ in a 50 mL round-bottom flask. The mixture was hydrogenated at room temperature for 16 h under hydrogen atmosphere using a hydrogen balloon, filtered through a Celite pad and concentrated under reduced pressure to afford tert-butyl N-[(1R,5S)-8-azabicyclo[3.2.1]octan-3-yl]-N-cyclopropyl-carbamate (370 mg, 1.39 mmol, 57% yield) as an oil. LCMS (ES, m/z): 267 [M+H]⁺.

An analogous method was followed to obtain the following compounds.

| | Starting | |
|---|---|---|
| Compound | Material | Characterization |
| 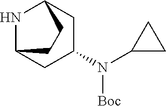 [SMILES: CC(C)(C)OC(=O)N(C1CC1)[C@@H]1C[C@H]2CC[C@@H](C1)N2] | 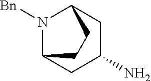 [SMILES: N[C@@H]1C[C@H]2CC[C@@H](C1)N2Cc1ccccc1] | Modification: Step 1 was ran for 8 h at 50° C. in DCE. Step 2 was ran in DCM and TEA. Step 3 was ran for 16 h at 50° C. This resulted in tert-butyl N-[(1S,5R)-8- azabicyclo[3.2.1]octan-3-yl]-N-cyclopropyl-carbamate (C148, 210 mg, 788.4 μmol, 94% yield) as a solid. LCMS (ES, m/z): 267 [M + H]⁺. |
| C148 | | |

### Example 156: Synthesis of Compound 448

### Synthesis of B136

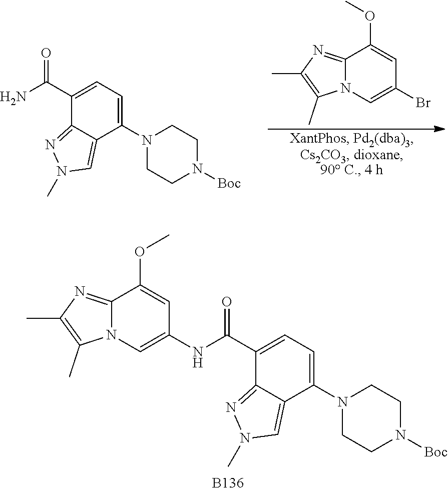

To a stirred mixture of tert-butyl 4-(7-carbamoyl-2-methyl-indazol-4-yl) piperazine-1-carboxylate (700 mg, 1.95 mmol) and 6-bromo-8-methoxy-2,3-dimethyl-imidazo[1,2-a]pyridine (451.6 mg, 1.77 mmol) in dioxane (7 mL) were added Cs₂CO₃ (1.73 g, 5.31 mmol), Xantphos (204.8 mg, 354.1 μmol) and Pd₂(dba)₃ (162.1 mg, 177.1 μmol). The resulting mixture was stirred for 4 h at 90° C. under N₂. The resulting mixture was concentrated under reduced pressure and purified by silica gel column chromatography (9% MeOH in DCM) to afford tert-butyl 4-[7-[(8-methoxy-2,3-dimethyl-imidazo[1,2-a]pyridin-6-yl)carbamoyl]-2-methyl-indazol-4-yl]piperazine-1-carboxylate (817 mg, 1.53 mmol, 87% yield) as a solid. LCMS (ES, m/z): 534 [M+H]⁺.

### Synthesis of B137

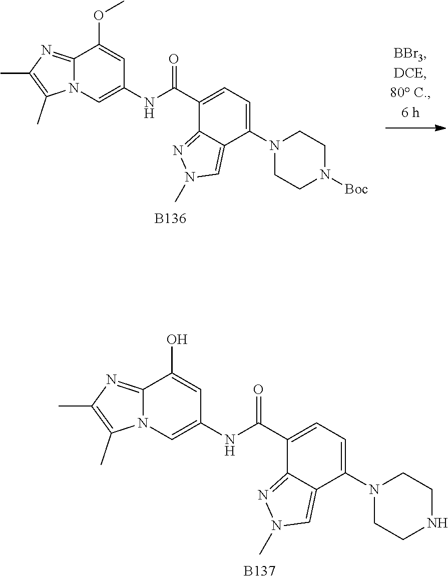 [SMILES: "B136" ; "B137" ; COc1cc(NC(=O)c2ccc(N3CCN(C(=O)OC(C)(C)C)CC3)c3cn(C)nc23)cn2c(C)c(C)nc12 ; Cc1nc2c(O)cc(NC(=O)c3ccc(N4CCNCC4)c4cn(C)nc34)cn2c1C]

To a stirred solution of tert-butyl 4-[7-[(8-methoxy-2,3-dimethyl-imidazo[1,2-a]pyridin-6-yl)carbamoyl]-2-methyl-indazol-4-yl]piperazine-1-carboxylate (817 mg, 1.53 mmol) in DCE (8 mL) was added tribromoborane (1.15 g, 4.59 mmol) dropwise at 0° C. The resulting mixture was stirred for 6 h at 80° C. The reaction was quenched with MeOH (5.0 mL) at 0° C. The resulting mixture was concentrated under reduced pressure to afford N-(8-hydroxy-2,3-dimethyl-imidazo[1,2-a]pyridin-6-yl)-2-methyl-4-piperazin-1-yl-indazole-7-carboxamide (2 g, crude) which was used in the next step directly without further purification. LCMS (ES, m/z): 420 [M+H]⁺.

### Synthesis of B138

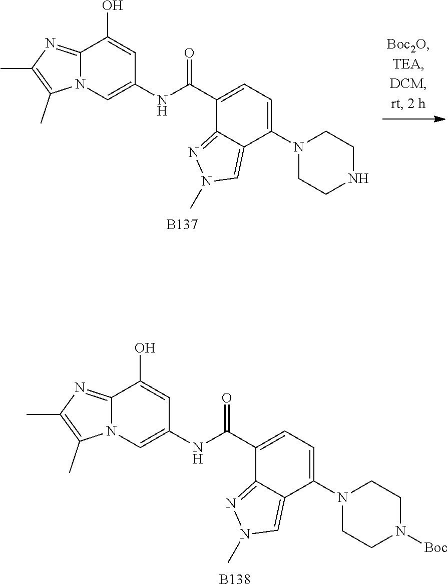 [SMILES: "B137" ; Cc1nc2c(O)cc(NC(=O)c3ccc(N4CCNCC4)c4cn(C)nc34)cn2c1C ; "B138" ; Cc1nc2c(O)cc(NC(=O)c3ccc(N4CCN(C(=O)OC(C)(C)C)CC4)c4cn(C)nc34)cn2c1C]

To a stirred solution of N-(8-hydroxy-2,3-dimethyl-imidazo[1,2-a]pyridin-6-yl)-2-methyl-4-piperazin-1-yl-indazole-7-carboxamide (2 g, 4.77 mmol) in DCM (20 mL) were added TEA (2.41 g, 23.84 mmol, 3.32 mL) and Boc₂O (3.12 g, 14.3 mmol, 3.28 mL). The resulting mixture was stirred for 2 h at room temperature. The resulting mixture was concentrated under reduced pressure. The resulting mixture was diluted with MeOH (20 mL). To the above mixture was added K₂CO₃ (2.0 g). The resulting mixture was stirred for 30 min at room temperature. The mixture was concentrated under reduced pressure and purified by silica gel column chromatography (9% MeOH in DCM) to afford tert-butyl 4-[7-[(8-hydroxy-2,3-dimethyl-imidazo[1,2-a]pyridin-6-yl)carbamoyl]-2-methyl-indazol-4-yl]piperazine-1-carboxylate (360 mg, 692.9 μmol, 15% yield) as a solid. LCMS (ES, m/z): 520 [M+H]⁺.

### Synthesis of B139

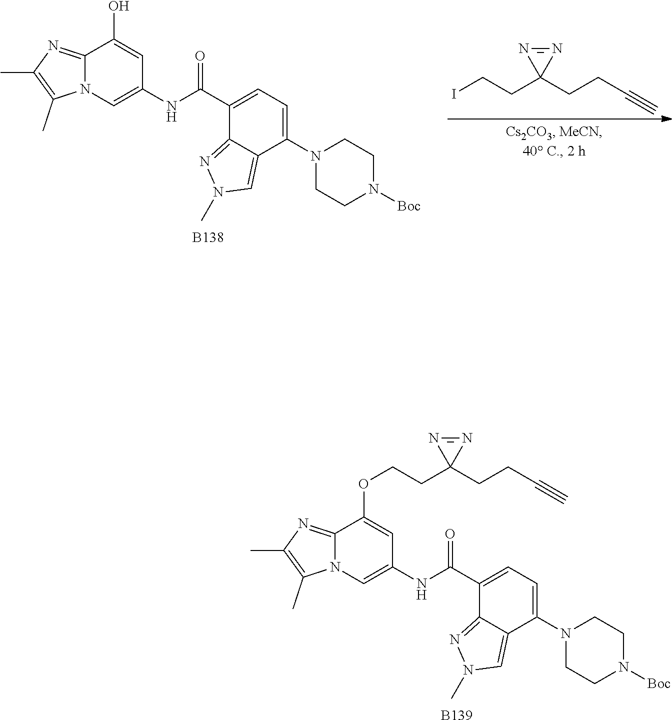

To a stirred mixture of tert-butyl 4-[7-[(8-hydroxy-2,3-dimethyl-imidazo[1,2-a]pyridin-6-yl)carbamoyl]-2-methyl-indazol-4-yl]piperazine-1-carboxylate (180 mg, 346.4 μmol) and 3-but-3-ynyl-3-(2-iodoethyl)diazirine (128.9 mg, 519.6 μmol) in MeCN (4 mL) was added Cs₂CO₃ (225.7 mg, 692.9 μmol). The resulting mixture was stirred for 2 h at 40° C. The mixture was purified by reverse phase flash chromatography (Condition 4, Gradient 6) to afford tert-butyl 4-[7-[[8-[2-(3-but-3-ynyldiazirin-3-yl) ethoxy]-2,3-dimethyl-imidazo[1,2-a]pyridin-6-yl]-carbamoyl]-2-methyl-indazol-4-yl]piperazine-1-carboxylate (64 mg, 100 μmol, 29% yield) as an oil. LCMS (ES, m/z): 640 [M+H]⁺.

### Synthesis of Compound 448

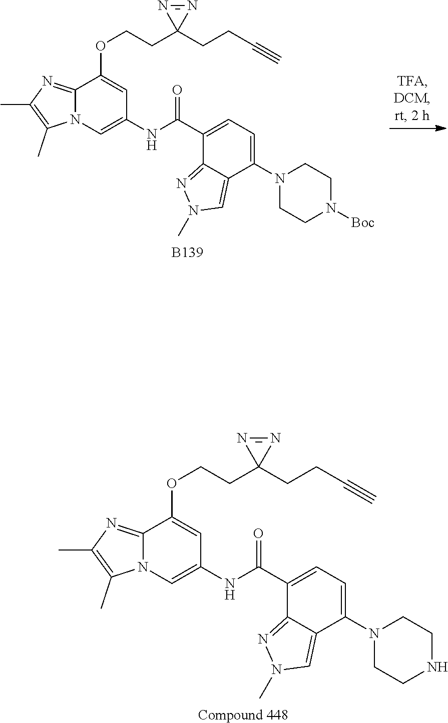

To a stirred solution of tert-butyl 4-[7-[[8-[2-(3-but-3-ynyldiazirin-3-yl) ethoxy]-2,3-dimethyl-imidazo[1,2-a]pyridin-6-yl]carbamoyl]-2-methyl-indazol-4-yl]piperazine-1-carboxylate (64 mg, 100.0 μmol) in DCM (1.2 mL) was added TFA (0.6 mL). The resulting mixture was stirred for 2 h at room temperature. The resulting mixture was concentrated under reduced pressure and purified by Prep-HPLC (Condition 16, Gradient 2) to afford N-(8-(2-(3-(but-3-yn-1-yl)-3H-diazirin-3-yl) ethoxy)-2,3-dimethylimidazo[1,2-a]pyridin-6-yl)-2-methyl-4-(piperazin-1-yl)-2H-indazole-7-carboxamide 2,2,2-trifluoroacetate (Compound 448, 7.8 mg, 11.9 μmol, 12% yield) as a solid. LCMS (ES, m/z): 540 [M+H]⁺. ¹H NMR (300 MHz, DMSO-d₆) δ 14.42 (s, 1H), 11.24 (s, 1H), 9.01 (s, 1H), 8.94 (s, 1H), 8.91-8.86 (m, 1H), 8.05 (d, J=8.0 Hz, 1H), 7.50 (s, 1H), 6.64 (d, J=8.2 Hz, 1H), 4.34 (s, 3H), 4.28 (t, J=6.2 Hz, 2H), 3.62 (t, J=5.1 Hz, 4H), 3.52 (m, 4H), 2.86 (t, J=2.6 Hz, 1H), 2.49 (s, 3H), 2.47 (s, 3H), 2.07 (ddd, J=7.1, 5.4, 2.8 Hz, 4H), 1.77 (t, J=7.3 Hz, 2H).

### Example 157: Synthesis of Compound 454

### Synthesis of B140

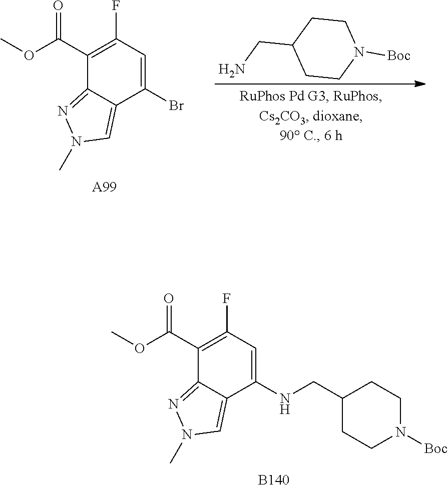

To a stirred mixture of methyl 4-bromo-6-fluoro-2-methyl-indazole-7-carboxylate (A99, 600 mg, 2.09 mmol) and tert-butyl 4-(aminomethyl) piperidine-1-carboxylate (537.4 mg, 2.51 mmol) in Dioxane (12 mL) were added Cs₂CO₃ (1.36 g, 4.18 mmol), RuPhos Pd G3 (174.8 mg, 209.0 μmol) and RuPhos (195.0 mg, 418 μmol) at room temperature under N₂. The resulting mixture was stirred for 6 h at 90° C. under N₂. The resulting mixture was concentrated under reduced pressure and purified by silica gel column chromatography (75% EtOAc in PE) to afford methyl 4-[(1-tert-butoxycarbonyl-4-piperidyl)methylamino]-6-fluoro-2-methyl-indazole-7-carboxylate (B140, 700 mg, 1.66 mmol, 80% yield) as a solid. LCMS (ES, m/z): 421 [M+H]⁺.

### Synthesis of B141

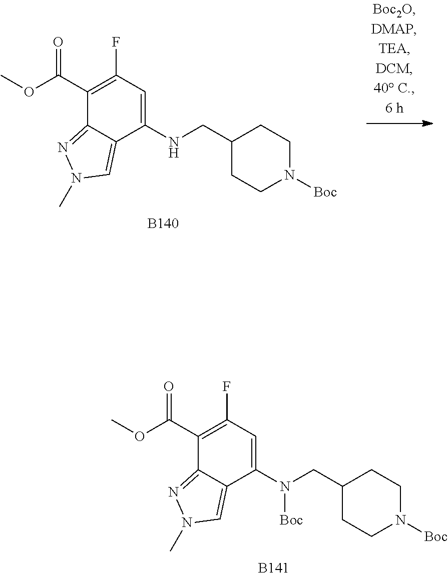 [SMILES: "B140" ; "B141" ; COC(=O)c1c(F)cc(NCC2CCN(C(=O)OC(C)(C)C)CC2)c2cn(C)nc12 ; COC(=O)c1c(F)cc(N(CC2CCN(C(=O)OC(C)(C)C)CC2)C(=O)OC(C)(C)C)c2cn(C)nc12]

To a stirred mixture of methyl 4-[(1-tert-butoxycarbonyl-4-piperidyl)methylamino]-6-fluoro-2-methyl-indazole-7-carboxylate (700 mg, 1.66 mmol) in DCM (14 mL) were added TEA (421.1 mg, 4.16 mmol), DMAP (20.3 mg, 166.5 μmol) and Boc₂O (545.0 mg, 2.50 mmol) at room temperature under N₂. The resulting mixture was stirred for 6 h at 40° C. under N₂. The mixture was diluted with DCM (100 mL) and washed with water (2×100 mL), dried over anhydrous Na₂SO₄, and filtered. The filtrate was concentrated under reduced pressure and purified by silica gel column chromatography (67% EtOAc in PE) to afford methyl 4-[tert-butoxycarbonyl-[(1-tert-butoxycarbonyl-4-piperidyl)methyl]amino]-6-fluoro-2-methyl-indazole-7-carboxylate (750 mg, 1.44 mmol, 87% yield) as a solid. LCMS (ES, m/z): 521 [M+H]⁺.

### Synthesis of B142

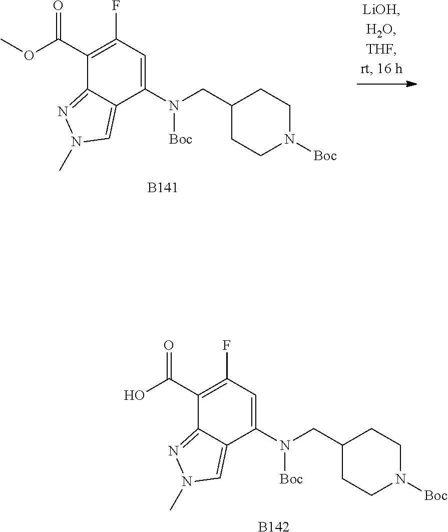 [SMILES: "B141" ; "B142" ; COC(=O)c1c(F)cc(N(CC2CCN(C(=O)OC(C)(C)C)CC2)C(=O)OC(C)(C)C)c2cn(C)nc12 ; Cn1cc2c(N(CC3CCN(C(=O)OC(C)(C)C)CC3)C(=O)OC(C)(C)C)cc(F)c(C(=O)O)c2n1]

To a stirred mixture of methyl 4-[tert-butoxycarbonyl-[(1-tert-butoxycarbonyl-4-piperidyl)methyl]amino]-6-fluoro-2-methyl-indazole-7-carboxylate (730 mg, 1.40 mmol) in THF (7 mL), H₂O (7 mL) was treated with LiOH (100.7 mg, 4.21 mmol) at 0° C. The resulting mixture was stirred for 16 h at room temperature. The resulting mixture was diluted with water (50 mL). The mixture was neutralized to pH 5 with HCl (aq.). The aqueous layer was extracted with EtOAc (2×50 mL). The combined organic layer was dried by anhydrous Na₂SO₄, and filtered. The filtrate was concentrated under reduced pressure, to afford 4-[tert-butoxycarbonyl-[(1-tert-butoxycarbonyl-4-piperidyl)methyl]amino]-6-fluoro-2-methyl-indazole-7-carboxylic acid (600 m5 g, 1.18 mmol, 85% yield) as a solid. LCMS (ES, m/z): 438 [M+H]⁺.

### Synthesis of B143

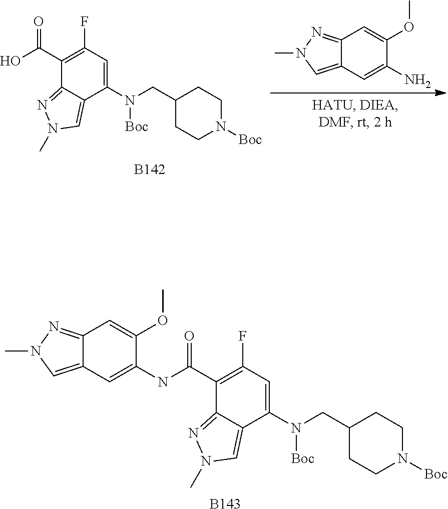 [SMILES: "B142" ; "B143" ; COc1cc2nn(C)cc2cc1NC(=O)c1c(F)cc(N(CC2CCN(C(=O)OC(C)(C)C)CC2)C(=O)OC(C)(C)C)c2cn(C)nc12 ; Cn1cc2c(N(CC3CCN(C(=O)OC(C)(C)C)CC3)C(=O)OC(C)(C)C)cc(F)c(C(=O)O)c2n1 ; COc1cc2nn(C)cc2cc1N]

To a stirred mixture of 4-[tert-butoxycarbonyl-[(1-tert-butoxycarbonyl-4-piperidyl)-methyl]amino]-6-fluoro-2-methyl-indazole-7-carboxylic acid (100 mg, 197.4 μmol) and 6-methoxy-2-methyl-indazol-5-amine (41.9 mg, 236.9 μmol) in DMF (2 mL) were added DIEA (102.0 mg, 789.6 μmol) and HATU (112.5 mg, 296.1 μmol), and the reaction was stirred for 2 h at room temperature. The resulting mixture was diluted with water (10 mL) and extracted with EtOAc (2×10 mL). The combined organic layers were washed with water (2×50 mL), dried over anhydrous Na₂SO₄, and filtered. The filtrate was concentrated under reduced pressure and purified by silica gel column chromatography (67% EtOAc in PE) to afford tert-butyl 4-[[tert-butoxycarbonyl-[6-fluoro-7-[(6-methoxy-2-methyl-indazol-5-yl)carbamoyl]-2-methyl-indazol-4-yl]amino]methyl]-piperidine-1-carboxylate (80 mg, 120.2 μmol, 61% yield) as a solid. LCMS (ES, m/z): 438 [M+H]⁺.

### Synthesis of Compound 454

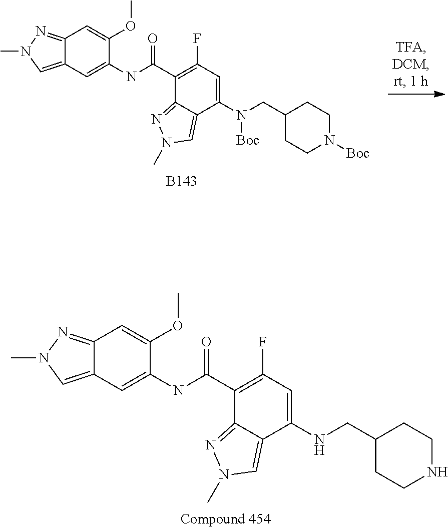

To a stirred solution of tert-butyl 4-[[tert-butoxycarbonyl-[6-fluoro-7-[(6-methoxy-2-methyl-indazol-5-yl)carbamoyl]-2-methyl-indazol-4-yl]amino]methyl]piperidine-1-carboxylate (80 mg, 120.2 μmol) in DCM (2 mL) was added TFA (1 mL) and the reaction was stirred at rt for 1 h. The resulting mixture was concentrated under reduced pressure and purified by Prep-HPLC (Condition 6, Gradient 6) to afford 6-fluoro-N-(6-methoxy-2-methyl-indazol-5-yl)-2-methyl-4-(4-piperidylmethylamino)indazole-7-carboxamide (Compound 454, 16.9 mg, 36.3 μmol, 30% yield) as a solid. LCMS (ES, m/z): 438 [M+H]⁺. ¹H NMR (400 MHz, DMSO-d₆) δ 11.44 (s, 1H), 8.78 (s, 1H), 8.60 (s, 1H), 8.15 (s, 1H), 7.34 (d, J=5.7 Hz, 1H), 7.04 (s, 1H), 5.87 (d, J=15.9 Hz, 1H), 4.22 (s, 3H), 4.08 (s, 3H), 4.02 (s, 3H), 3.11 (t, J=6.0 Hz, 2H), 2.95 (d, J=12.1 Hz, 2H), 2.45 (t, J=11.6 Hz, 2H), 1.75-1.67 (m, 3H), 1.17-1.07 (m, 2H).

### Example 158: Synthesis of Compound 389

### Synthesis of B144

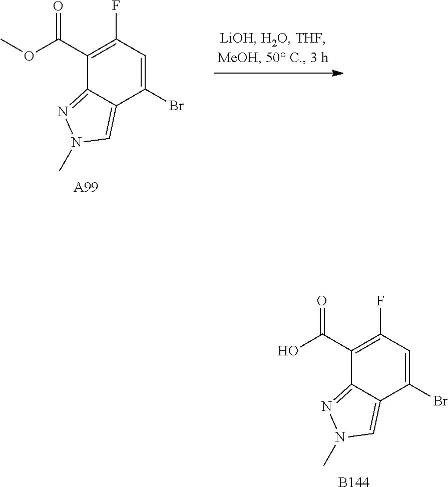

To a solution of methyl 4-bromo-6-fluoro-2-methyl-indazole-7-carboxylate (1 g, 3.48 mmol) in THF (8 mL)/H₂O (4 mL)/MeOH (4 mL) was added LiOH·H₂O (730.8 mg, 17.42 mmol), and the reaction was stirred for 3 h at 50° C. The resulting mixture was diluted with water and adjusted to pH 5-6 with 1 N HCl. The resulting mixture was extracted with DCM (2×50 mL). The combined organic layers were washed with water (2×80 mL), dried over anhydrous Na₂SO₄, and filtered. The filtrate was concentrated under reduced pressure to afford 4-bromo-6-fluoro-2-methyl-indazole-7-carboxylic acid (677 mg, 2.48 mmol, 71% yield) as a solid. LCMS (ES, m/z): 273 [M+H]⁺.

### Synthesis of B145

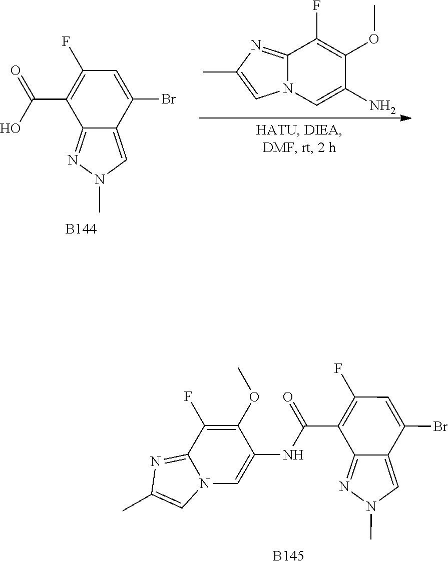

To a solution of 4-bromo-6-fluoro-2-methyl-indazole-7-carboxylic acid (320 mg, 1.17 mmol) in DMF (4 mL) were added 8-fluoro-7-methoxy-2-methyl-imidazo[1,2-a]pyridin-6-amine (274.5 mg, 1.41 mmol), DIEA (605.8 mg, 4.69 mmol) and HATU (668.4 mg, 1.76 mmol), and the reaction was stirred for 2 h at rt. The resulting mixture was diluted with H₂O (10 mL). The precipitated solids were collected by filtration, washed with H₂O (10 mL), MeCN (1 mL) and dried by air to afford 4-bromo-6-fluoro-N-(8-fluoro-7-methoxy-2-methyl-imidazo[1,2-a]pyridin-6-yl)-2-methyl-indazole-7-carboxamide (520 mg, 1.15 mmol, 99% yield) as a solid. LCMS (ES, m/z): 450 [M+H]⁺.

### Synthesis of Compound 389

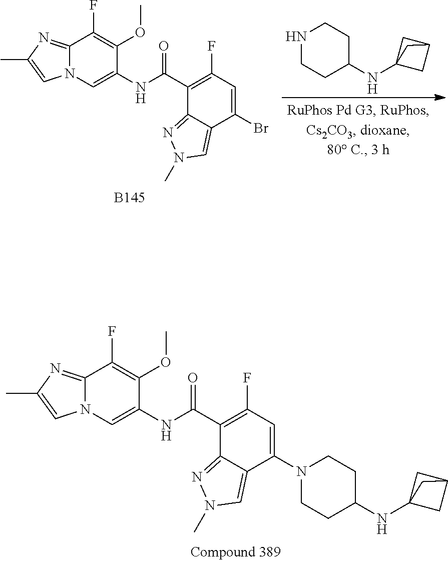 [SMILES: "B145" ; COc1c(NC(=O)c2c(F)cc(Br)c3cn(C)nc23)cn2cc(C)nc2c1F ; COc1c(NC(=O)c2c(F)cc(N3CCC(NC45CC(C4)C5)CC3)c3cn(C)nc23)cn2cc(C)nc2c1F ; C1CC(NC23CC(C2)C3)CCN1]

To a stirred mixture of 4-bromo-6-fluoro-N-(8-fluoro-7-methoxy-2-methyl-imidazo[1,2-a]pyridin-6-yl)-2-methyl-indazole-7-carboxamide (110 mg, 244.3 μmol) in dioxane (1.5 mL) were added N-(1-bicyclo[1.1.1]pentanyl)piperidin-4-amine (48.7 mg, 293.2 μmol), Cs₂CO₃ (159.2 mg, 488.6 μmol), Ruphos (22.8 mg, 48.86 μmol) and RuPhos Pd G3 (20.4 mg, 24.43 μmol), and the reaction was stirred for 3 h at 80° C. under N₂. The resulting mixture was diluted with H₂O (15 mL) and extracted with DCM (2×15 mL). The combined organic layers were washed with H₂O (2×20 mL), dried over anhydrous Na₂SO₄, and filtered. The filtrate was concentrated under reduced pressure and purified by silica gel column chromatography (50% EtOAc in DCM) and Prep-HPLC (Condition 5, Gradient 21) to afford 4-[4-(1-bicyclo[1.1.1]pentanylamino)-1-piperidyl]-6-fluoro-N-(8-fluoro-7-methoxy-2-methylimidazo-[1,2-a]pyridin-6-yl)-2-methyl-indazole-7-carboxamide (Compound 389, 15 mg, 28 μmol, 12% yield) as a solid. LCMS (ES, m/z): 536 [M+H]⁺. ¹H NMR (400 MHz, DMSO-d₆) δ 11.54 (s, 1H), 9.44 (d, J=1.2 Hz, 1H), 8.81 (s, 1H), 7.74 (dd, J=3.2, 1.1 Hz, 1H), 6.20 (d, J=16.2 Hz, 1H), 4.21 (s, 3H), 4.16 (d, J=2.0 Hz, 3H), 3.87 (d, J=13.1 Hz, 2H), 3.14 (t, J=11.5 Hz, 2H), 2.79 (dt, J=9.7, 5.5 Hz, 1H), 2.32 (s, 1H), 2.29 (d, J=0.9 Hz, 3H), 1.94-1.85 (m, 2H), 1.75 (s, 6H), 1.40 (q, J=11.3, 9.7 Hz, 2H).

### Example 159: Synthesis of C153

### Synthesis of C149

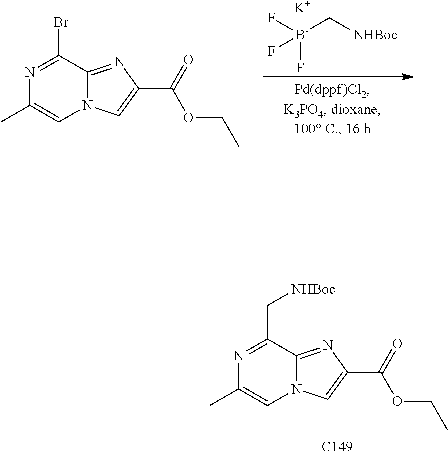 [SMILES: [K+] ; CCOC(=O)c1cn2cc(C)nc(Br)c2n1 ; CC(C)(C)OC(=O)NC[B-](F)(F)F ; CCOC(=O)c1cn2cc(C)nc(CNC(=O)OC(C)(C)C)c2n1 ; "C149"]

To a stirred mixture of methyl 8-bromo-6-methyl-imidazo[1,2-a]pyrazine-2-carboxylate (3.6 g, 13.33 mmol) and potassium (((tert-butoxycarbonyl)amino)methyl)trifluoroborate (3.96 g, 19.99 mmol) in dioxane (70 mL) were added water (7 mL) and K₃PO₄ (8.49 g, 39.99 mmol) and Pd(dppf)Cl₂ (975 mg, 1.33 mmol) at room temperature under N₂. The resulting mixture was stirred for 16 h at 100° C. under N₂. The mixture was allowed to cool down to room temperature. The resulting mixture was concentrated under reduced pressure and purified by silica gel column chromatography (33% EtOAc in PE) to afford methyl 8-[(tert-butoxycarbonylamino)methyl]-6-methyl-imidazo[1,2-a]pyrazine-2-carboxylate (1.8 g, 5.62 mmol, 42% yield) as a solid. LCMS (ES, m/z): 335 [M+H]⁺.

### Synthesis of C150

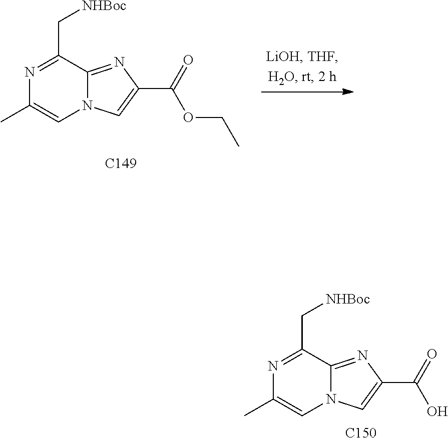 [SMILES: CCOC(=O)c1cn2cc(C)nc(CNC(=O)OC(C)(C)C)c2n1 ; "C149" ; Cc1cn2cc(C(=O)O)nc2c(CNC(=O)OC(C)(C)C)n1 ; "C150"]

To a stirred mixture of ethyl 8-[(tert-butoxycarbonylamino)methyl]-6-methyl-imidazo[1,2-a]pyrazine-2-carboxylate (1.8 g, 5.38 mmol) in THF (18 mL) were added water (18 mL) and LiOH (645 mg, 26.9 mmol), and the reaction was stirred for 2 h at room temperature. The resulting mixture was neutralized to PH 5 and the crude product was purified by Prep-HPLC (Condition 17, Gradient 1) to afford 8-[(tert-butoxycarbonylamino)methyl]-6-methyl-imidazo-[1,2-a]pyrazine-2-carboxylic acid (1.3 g, 4.24 mmol, 79% yield) as a solid. LCMS (ES, m/z): 307 [M+H]⁺.

### Synthesis of C151

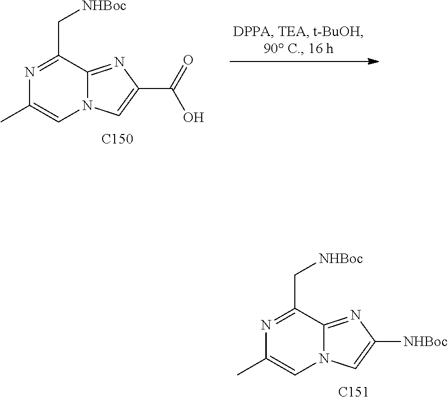 [SMILES: Cc1cn2cc(C(=O)O)nc2c(CNC(=O)OC(C)(C)C)n1 ; "C150" ; Cc1cn2cc(NC(=O)OC(C)(C)C)nc2c(CNC(=O)OC(C)(C)C)n1 ; "C151"]

To a stirred mixture of 8-[(tert-butoxycarbonylamino)methyl]-6-methyl-imidazo[1,2-a]pyrazine-2-carboxylic acid (1.3 g, 4.24 mmol) in t-BuOH (26 mL) were added TEA (644 mg, 6.37 mmol) and DPPA (1.75 g, 6.37 mmol, 1.4 mL) at room temperature under N₂. The resulting mixture was stirred for 16 h at 90° C. under N₂. The resulting mixture was diluted with water (30 mL) and extracted with EtOAc (3×30 mL). The combined organic layers were washed with water (2×100 mL) and brine (100 mL), dried over anhydrous Na₂SO₄, and filtered. The filtrate was concentrated under reduced pressure and purified by silica gel column chromatography (33% EtOAc in PE) to afford tert-butyl N-[[2-(tert-butoxycarbonylamino)-6-methyl-imidazo[1,2-a]pyrazin-8-yl]methyl]carbamate (350 mg, 927.3 μmol, 22% yield) as a solid. LCMS (ES, m/z): 378 [M+H]⁺.

### Synthesis of C152

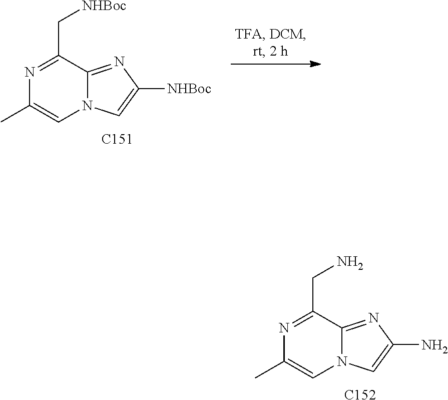 [SMILES: Cc1cn2cc(NC(=O)OC(C)(C)C)nc2c(CNC(=O)OC(C)(C)C)n1 ; "C151" ; Cc1cn2cc(N)nc2c(CN)n1 ; "C152"]

To a stirred mixture of tert-butyl N-[[2-(tert-butoxycarbonylamino)-6-methyl-imidazo[1,2-a]pyrazin-8-yl]methyl]carbamate (170 mg, 450.4 μmol) in DCM (4 mL) was added TFA (1 mL) dropwise, and the reaction was stirred for 2 h at room temperature. The resulting mixture was concentrated under reduced pressure and the crude product 8-(aminomethyl)-6-methyl-imidazo[1,2-a]pyrazin-2-amine (150 mg, 423.2 μmol, 94% yield, 50% purity) was used for the next step without further purification. LCMS (ES, m/z): 178 [M+H]⁺.

### Synthesis of C153

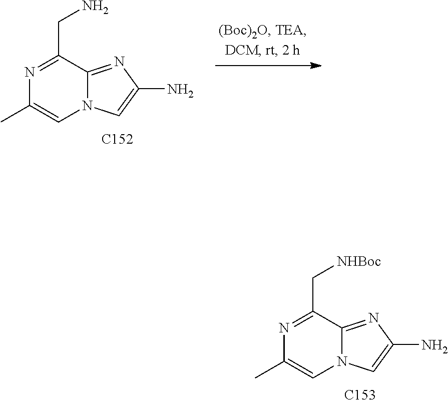 [SMILES: Cc1cn2cc(N)nc2c(CN)n1 ; "C152" ; Cc1cn2cc(N)nc2c(CNC(=O)OC(C)(C)C)n1 ; "C153"]

To a stirred mixture of 8-(aminomethyl)-6-methyl-imidazo[1,2-a]pyrazin-2-amine (150 mg, 423.2 μmol) in DCM (3 mL) were added TEA (129 mg, 1.27 mmol) and (Boc)₂O (102 mg, 465.6 μmol), and the reaction was stirred for 2 h at room temperature. The resulting mixture was diluted with water (8 mL) and extracted with DCM (3×10 mL). The combined organic layers were washed with water (2×20 mL) and brine (20 mL), dried over anhydrous Na₂SO₄, and filtered. The filtrate was concentrated under reduced pressure and purified by TLC to afford tert-butyl N-[(2-amino-6-methyl-imidazo[1,2-a]pyrazin-8-yl)methyl]carbamate (70 mg, 252.4 μmol, 60% yield) as a solid. LCMS (ES, m/z): 278 [M+H]⁺.

### Example 160: Synthesis of C154

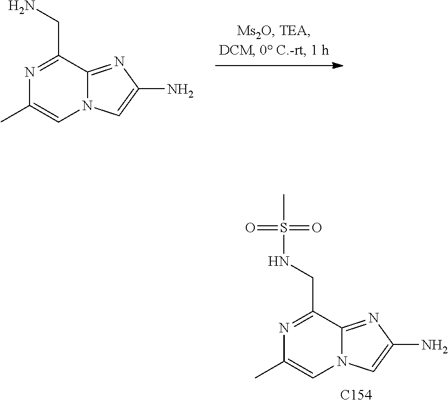 [SMILES: Cc1cn2cc(N)nc2c(CN)n1 ; Cc1cn2cc(N)nc2c(CNS(C)(=O)=O)n1 ; "C154"]

To a stirred mixture of tert-butyl N-[[2-(tert-butoxycarbonylamino)-6-methyl-imidazo[1,2-a]pyrazin-8-yl]methyl]carbamate (200 mg, 529.9 μmol) in DCM (4 mL) was added TEA (268 mg, 2.65 mmol) and Ms₂O (92 mg, 529.9 μmol) at 0° C. The resulting mixture was stirred for 1 h at room temperature. The resulting mixture was concentrated under reduced pressure and purified by silica gel column chromatography (9% MeOH in DCM) to afford N-[(2-amino-6-methyl-imidazo[1,2-a]pyrazin-8-yl)methyl]methanesulfonamide (15 mg, 58.8 μmol, 11% yield) as a solid. LCMS (ES, m/z): 256 [M+H]⁺.

### Example 161: Synthesis of C155

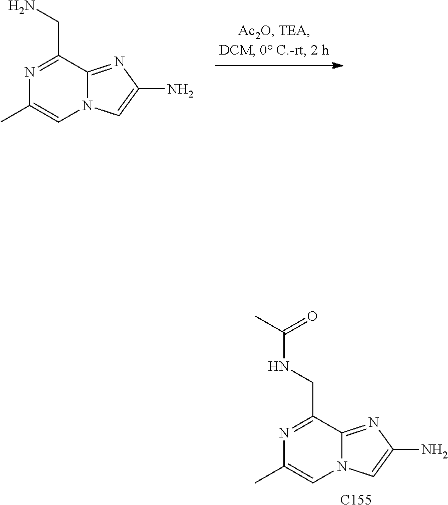 [SMILES: Cc1cn2cc(N)nc2c(CN)n1 ; CC(=O)NCc1nc(C)cn2cc(N)nc12 ; "C155"]

To a stirred mixture of tert-butyl N-[[2-(tert-butoxycarbonylamino)-6-methyl-imidazo[1,2-a]pyrazin-8-yl]methyl]carbamate (200 mg, 529.9 μmol) in DCM (4 mL) was added TEA (268 mg, 2.65 mmol) and (AcO)₂O (54 mg, 529.9 μmol) at 0° C. The resulting mixture was stirred for 2 h at room temperature. The resulting mixture was concentrated under reduced pressure and purified by silica gel column chromatography (9% MeOH in DCM) to afford N-[(2-amino-6-methyl-imidazo[1,2-a]pyrazin-8-yl)methyl]acetamide (20 mg, 91.2 μmol, 17% yield) as a solid. LCMS (ES, m/z): 220 [M+H]⁺.

### Example 162: Synthesis of Compound 401

### Synthesis of B146

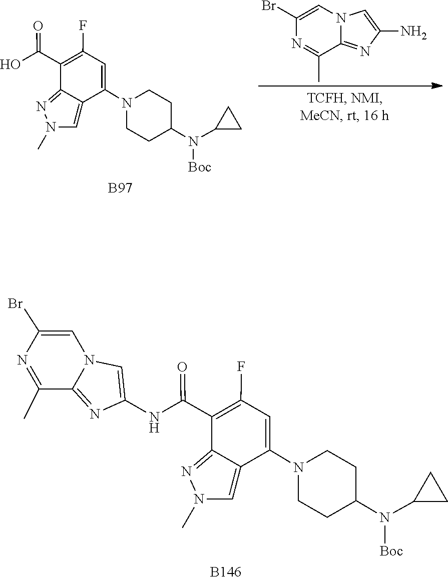

To a stirred mixture of 4-[4-[tert-butoxycarbonyl(cyclopropyl)amino]-1-piperidyl]-6-fluoro-2-methyl-indazole-7-carboxylic acid (360 mg, 832.4 μmol) and 6-bromo-8-methyl-imidazo[1,2-a]pyrazin-2-amine dihydrochloride (226.8 mg, 998.9 μmol) in MeCN (7 mL) were added TCFH (350.3 mg, 1.25 mmol) and NMI (273.3 mg, 3.33 mmol), and the reaction was stirred for 16 h at room temperature. The resulting mixture was extracted with EtOAc (10 mL), washed with water (2×10 mL), dried over anhydrous Na₂SO₄, and filtered. The filtrate was concentrated under reduced pressure and purified by silica gel column chromatography (67% EtOAc in PE) to afford tert-butyl N-[1-[7-[(6-bromo-8-methyl-imidazo[1,2-a]pyrazin-2-yl)carbamoyl]-6-fluoro-2-methyl-indazol-4-yl]-4-piperidyl]-N-cyclopropyl-carbamate (310 mg, 483.2 μmol, 58% yield) as a solid. LCMS (ES, m/z): 641 [M+H]⁺.

### Synthesis of B147

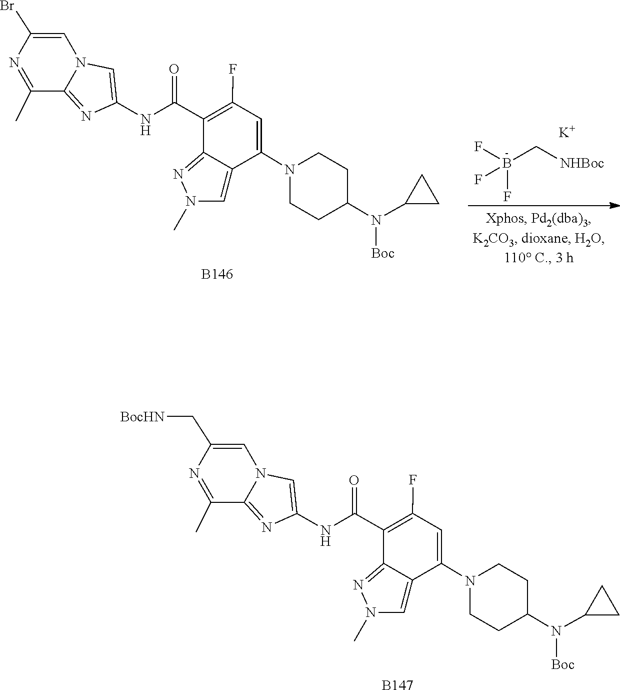

To a stirred mixture of tert-butyl N-[1-[7-[(6-bromo-8-methyl-imidazo[1,2-a]pyrazin-2-yl)carbamoyl]-6-fluoro-2-methyl-indazol-4-yl]-4-piperidyl]-N-cyclopropyl-carbamate (130 mg, 202.6 μmol) and potassium (((tert-butoxycarbonyl)amino)methyl)trifluoroborate (48.1 mg, 243.2 μmol) in Dioxane (2 mL) and H₂O (0.4 mL) were added K₂CO₃ (56.0 mg, 405.3 μmol), Xphos (19.3 mg, 40.53 μmol) and Pd₂(dba)₃ (18.5 mg, 20.26 μmol) at room temperature under N₂. The resulting mixture was stirred for 3 h at 110° C. under N₂. The resulting mixture was concentrated under reduced pressure and purified by silica gel column chromatography (75% EtOAc in PE) to afford tert-butyl N-[1-[7-[[6-[(tert-butoxycarbonylamino)methyl]-8-methyl-imidazo[1,2-a]-pyrazin-2-yl]carbamoyl]-6-fluoro-2-methyl-indazol-4-yl]-4-piperidyl]-N-cyclopropyl-carbamate (130 mg, 187.9 μmol, 93% yield) as a solid. LCMS (ES, m/z): 692 [M+H]⁺.

### Synthesis of Compound 401

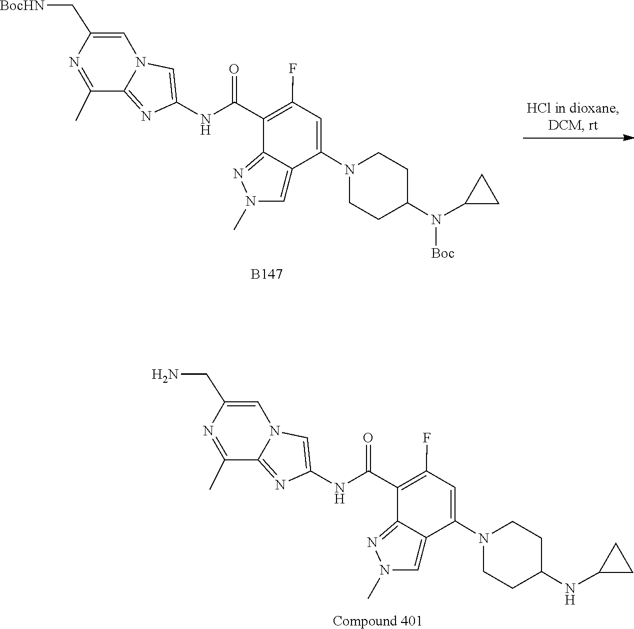

To a stirred solution of tert-butyl N-[1-[7-[[6-[(tert-butoxycarbonylamino)methyl]-8-methyl-imidazo[1,2-a]pyrazin-2-yl]carbamoyl]-6-fluoro-2-methyl-indazol-4-yl]-4-piperidyl]-N-cyclopropyl-carbamate (120 mg, 173.5 μmol) in DCM (2 mL) was added TFA (1.49 g, 13.07 mmol, 1 mL), and the reaction was stirred for 2 h at rt. The resulting mixture was concentrated under reduced pressure and purified by Prep-HPLC (Condition 6, Gradient 9) to afford N-[6-(aminomethyl)-8-methyl-imidazo[1,2-a]pyrazin-2-yl]-4-[4-(cyclopropylamino)-1-piperidyl]-6-fluoro-2-methyl-indazole-7-carboxamide (Compound 401, 5 mg, 10.2 μmol, 6% yield) as a solid. LCMS (ES, m/z): 492 [M+H]⁻. ¹H NMR (400 MHz, DMSO-d₆) δ 11.57 (s, 1H), 8.83 (s, 1H), 8.40 (s, 1H), 8.35 (s, 1H), 6.25 (d, J=16.0 Hz, 1H), 4.24 (s, 3H), 3.92 (d, J=13.2 Hz, 3H), 3.76 (s, 2H), 3.14 (t, J=12.0 Hz, 2H), 2.81 (t, J=9.4 Hz, 1H), 2.71 (s, 3H), 2.11 (dq, J=6.6, 3.3 Hz, 1H), 1.99 (d, J=13.0 Hz, 2H), 1.45 (q, J=9.8 Hz, 2H), 0.40 (dt, J=6.2, 3.0 Hz, 2H), 0.27-0.19 (m, 2H).

### Example 163: Synthesis of C157

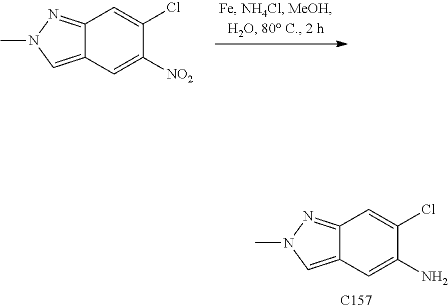

To a stirred mixture of 6-chloro-2-methyl-5-nitro-indazole (1 g, 4.73 mmol) in MeOH (20 mL) and H₂O (20 mL) were added NH₄Cl (1.52 g, 28.35 mmol) and Fe (1.06 g, 18.9 mmol), and the reaction was stirred for 2 h at 80° C. The resulting mixture was diluted with water (100 mL). The precipitated solids were filtered and the filtrate was extracted with EtOAc (2×100 mL), dried over anhydrous Na₂SO₄, and filtered. The filtrate was concentrated under reduced pressure and purified by silica gel column chromatography (50% EtOAc in PE) to afford 6-chloro-2-methyl-indazol-5-amine (C157, 500 mg, 2.75 mmol, 58% yield) as a solid. LCMS (ES, m/z): 182 [M+H]⁺.

### Example 164: Synthesis of Compound 613

### Synthesis of B149

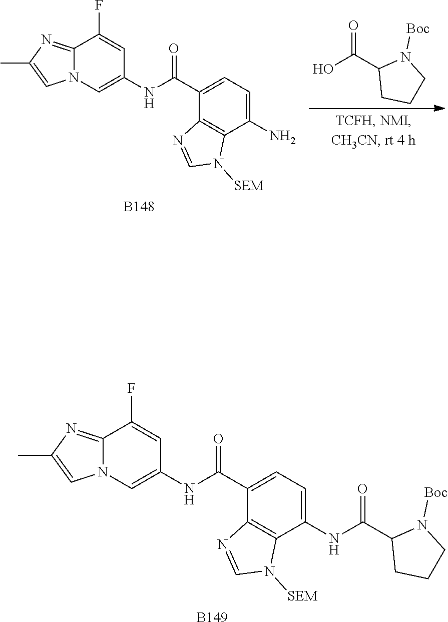 [SMILES: "B148" ; "B149" ; Cc1cn2cc(NC(=O)c3ccc(N)c4c3ncn4COCC[Si](C)(C)C)cc(F)c2n1 ; Cc1cn2cc(NC(=O)c3ccc(NC(=O)C4CCCN4C(=O)OC(C)(C)C)c4c3ncn4COCC[Si](C)(C)C)cc(F)c2n1 ; CC(C)(C)OC(=O)N1CCCC1C(=O)O]

To a stirred mixture of 7-amino-N-(8-fluoro-2-methyl-imidazo[1,2-a]pyridin-6-yl)-1-(2-trimethylsilylethoxymethyl)benzimidazole-4-carboxamide (100 mg, 220 μmol) and 1-tert-butoxycarbonylpyrrolidine-2-carboxylic acid (56 mg, 264 μmol) in MeCN (4.0 mL) was added NMI (18 mg, 220 μmol) and TCFH (61 mg, 220 μmol), and the reaction was stirred for 4 h at room temperature. The resulting mixture was diluted with water (10 mL). The resulting solid was collected by filtration and washed with water (5 mL) to afford tert-butyl 2-[[7-[(8-fluoro-2-methyl-imidazo[1,2-a]pyridin-6-yl)carbamoyl]-3-(2-trimethylsilylethoxymethyl)benzimidazol-4-yl]carbamoyl]pyrrolidine-1-carboxylate (70 mg, 107.4 μmol, 49% yield) as a solid. LCMS (ES, m/z): 652 [M+H]⁺.

### Synthesis of Compound 613

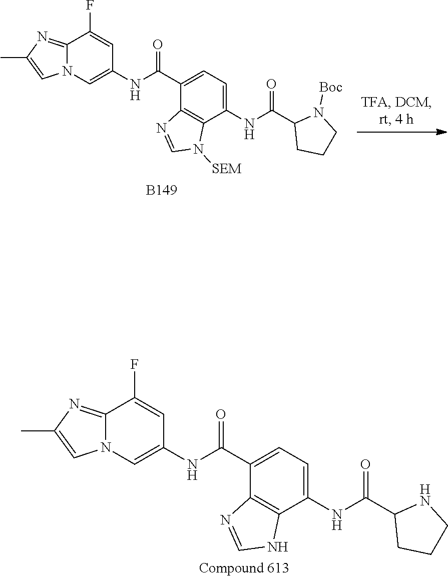 [SMILES: "B149" ; Cc1cn2cc(NC(=O)c3ccc(NC(=O)C4CCCN4C(=O)OC(C)(C)C)c4c3ncn4COCC[Si](C)(C)C)cc(F)c2n1 ; Cc1cn2cc(NC(=O)c3ccc(NC(=O)C4CCCN4)c4[nH]cnc34)cc(F)c2n1 ; "Compound 613"]

To a solution of tert-butyl 2-[[7-[(8-fluoro-2-methyl-imidazo[1,2-a]pyridin-6-yl)-carbamoyl]-3-(2-trimethylsilylethoxymethyl)benzimidazol-4-yl]carbamoyl]pyrrolidine-1-carboxylate (70 mg, 107.4 μmol) in DCM (3 mL) was added TFA (1 mL), and the reaction was stirred for 2 h at rt. The resulting mixture was concentrated under reduced pressure and adjusted to pH 8 with saturated NaHCO₃ (aq.). The resulting mixture was diluted with water (5 mL) and extracted with DCM/MeOH (20/1) (3×10 mL). The combined organic layers were washed with brine (5 mL) and dried over anhydrous Na₂SO₄, and filtered. The filtrate was concentrated under reduced pressure and purified by reversed-phase flash chromatography (Condition 3, Gradient 11) to afford N-(8-fluoro-2-methyl-imidazo[1,2-a]pyridin-6-yl)-7-(pyrrolidine-2-carbonylamino)-1H-benzimidazole-4-carboxamide (Compound 613, 10 mg, 23.7 μmol, 22% yield) as a solid. LCMS (ES, m/z): 422 [M+H]⁺. ¹H NMR (400 MHz, DMSO-d₆) δ 12.77 (s, 1H), 11.00 (s, 1H), 10.38 (s, 1H), 9.07 (s, 1H), 8.22 (s, 2H), 8.00-7.90 (m, 2H), 7.42 (d, J=12.8 Hz, 1H), 3.93 (s, 1H), 3.11-3.01 (m, 1H), 2.91 (s, 1H), 2.36 (s, 4H), 2.16 (s, 1H), 1.90 (s, 1H), 1.72 (s, 2H).

An analogous method was followed to obtain the following compounds.

| | Starting | |
|---|---|---|
| Compound | Material | Characterization |
| 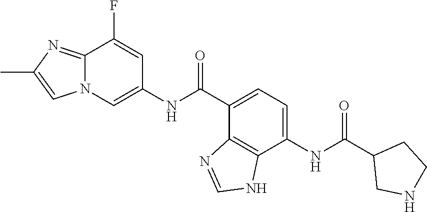 [SMILES: Cc1cn2cc(NC(=O)c3ccc(NC(=O)C4CCNC4)c4[nH]cnc34)cc(F)c2n1] | 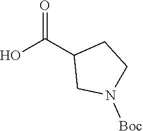 [SMILES: CC(C)(C)OC(=O)N1CCC(C(=O)O)C1] | Modification: Step 2 was ran for 2 h. The residue was purified by reversed- phase flash chromatography (Condition 3, Gradient 11) to afford N-(8-fluoro-2- methyl-imidazo[1,2-a]pyridin-6-yl)-7- (pyrrolidine-3-carbonylamino)-1H- benzimidazole-4-carboxamide (Compound 614, 20 mg, 47.5 μmol, 52% yield) as a solid. LCMS (ES, m/z): 422 [M + H]⁺. ¹H NMR (400 MHz, Methanol- d₄) δ 9.12 (d, J = 1.7 Hz, 1H), 8.33 (s, 1H), 7.99 (d, J = 8.3 Hz, 1H), 7.71 (d, J = 3.0 Hz, 1H), 7.65 (s, 1H), 7.31 (dd, J = |
| Compound 614 | | 12.0, 1.7 Hz, 1H), 3.32-3.18 (m, 4H), |
| | | 3.10 (dt, J = 11.4, 7.3 Hz, 1H), 2.44 (s, |
| | | 3H), 2.35-2.17 (m, 2H). |

### Example 165: Synthesis of Compound 616

### Synthesis of B150

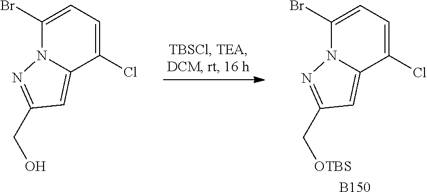 [SMILES: OCc1cc2c(Cl)ccc(Br)n2n1 ; "B150" ; CC(C)(C)[Si](C)(C)OCc1cc2c(Cl)ccc(Br)n2n1]

To a stirred solution of (7-bromo-4-chloro-pyrazolo[1,5-a]pyridin-2-yl) methanol (450 mg, 1.72 mmol) and TEA (261 mg, 2.58 mmol) in DCM (9 mL) was added TBSCl (778 mg, 5.16 mmol), and the reaction was stirred for 16 h at rt under N₂. The resulting mixture was diluted with water (10 mL) and extracted with EtOAc (3×10 mL). The combined organic layers were washed with water (2×10 mL) and brine (10 mL), dried over anhydrous Na₂SO₄, and filtered. The filtrate was concentrated under reduced pressure and purified by silica gel column chromatography (20% EtOAc in PE) to afford (7-bromo-4-chloro-pyrazolo[1,5-a]pyridin-2-yl)methoxy-tert-butyl-dimethyl-silane (553 mg, 1.47 mmol, 86% yield) as a solid. LCMS (ES, m/z): 375 [M+H]⁺.

### Synthesis of B151

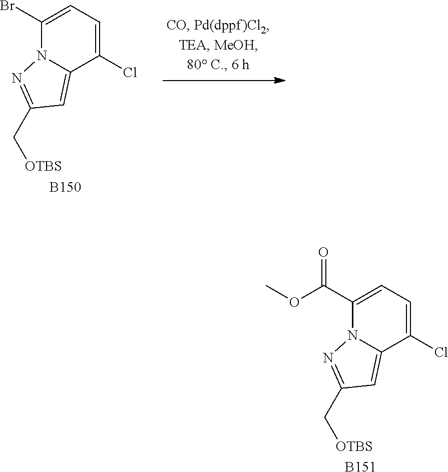 [SMILES: "B150" ; CC(C)(C)[Si](C)(C)OCc1cc2c(Cl)ccc(Br)n2n1 ; "B151" ; COC(=O)c1ccc(Cl)c2cc(CO[Si](C)(C)C(C)(C)C)nn12]

To a solution of (7-bromo-4-chloro-pyrazolo[1,5-a]pyridin-2-yl)methoxy-tert-butyl-dimethyl-silane (520 mg, 1.38 mmol) and TEA (420 mg, 4.15 mmol) in MeOH (10 mL) was added Pd(dppf)Cl₂ (101 mg, 138.4 μmol) in a pressure tank. The mixture was purged with nitrogen and then was pressurized to 20 atm with carbon monoxide at 80° C. for 6 hr. The reaction mixture was cooled to room temperature and filtered to remove insoluble solids. The resulting mixture was concentrated under reduced pressure to afford methyl 2-[[tert-butyl(dimethyl)silyl]oxymethyl]-4-chloro-pyrazolo[1,5-a]pyridine-7-carboxylate (440 mg, 1.24 mmol, 90% yield) as a solid. LCMS (ES, m/z): 355 [M+H]⁺.

### Synthesis of B152

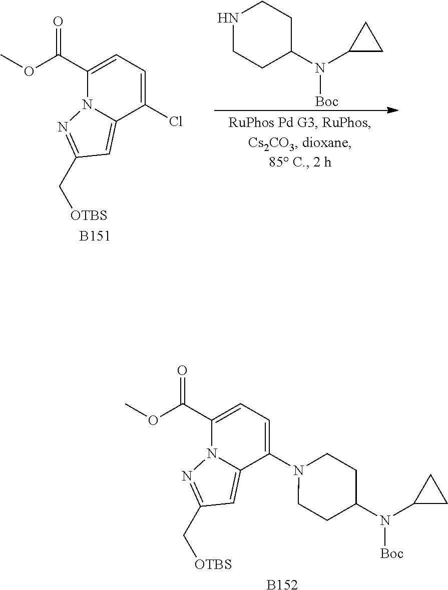 [SMILES: "B151" ; "B152" ; COC(=O)c1ccc(Cl)c2cc(CO[Si](C)(C)C(C)(C)C)nn12 ; COC(=O)c1ccc(N2CCC(N(C(=O)OC(C)(C)C)C3CC3)CC2)c2cc(CO[Si](C)(C)C(C)(C)C)nn12 ; CC(C)(C)OC(=O)N(C1CCNCC1)C1CC1]

To a stirred mixture of methyl 2-[[tert-butyl(dimethyl)silyl]oxymethyl]-4-chloro-pyrazolo[1,5-a]pyridine-7-carboxylate (420 mg, 1.18 mmol) and tert-butyl N-cyclopropyl-N-(4-piperidyl)carbamate (369 mg, 1.54 mmol) in Dioxane (8 mL) were added Cs₂CO₃ (1.16 g, 3.55 mmol), RuPhos (110 mg, 236.7 μmol) and RuPhos Pd G3 (98 mg, 118.3 μmol) at room temperature under N₂. The resulting mixture was stirred for 2 h at 85° C. under N₂. The resulting mixture was allowed to cool to room temperature, diluted with water (20 mL), and extracted with EtOAc (2×20 mL). The combined organic layers were washed with brine (20 mL), dried over anhydrous Na₂SO₄, and filtered. The filtrate was concentrated under reduced pressure and purified by silica gel column chromatography (50% EtOAc in PE) to afford methyl 4-[4-[tert-butoxycarbonyl(cyclopropyl)amino]-1-piperidyl]-2-[[tert-butyl(dimethyl)silyl]oxymethyl]-pyrazolo[1,5-a]pyridine-7-carboxylate (360 mg, 644.3 μmol, 54% yield) as a solid. LCMS (ES, m/z): 559 [M+H]⁺.

### Synthesis of B153

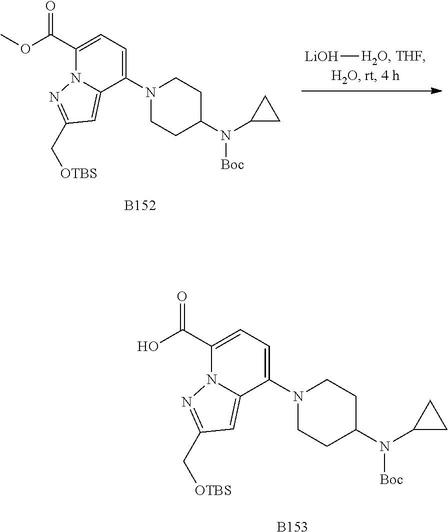

A solution of methyl 4-[4-[tert-butoxycarbonyl(cyclopropyl)amino]-1-piperidyl]-2-[[tert-butyl(dimethyl)silyl]oxymethyl]pyrazolo[1,5-a]pyridine-7-carboxylate (330 mg, 590.6 μmol) in THF (3 mL) and H₂O (3 mL) was treated with LiOH·H₂O (49 mg, 1.18 mmol), and the reaction was stirred for 4 h at room temperature under N₂. The mixture was acidified to pH 3 with HCl (1N) and extracted with EtOAc (2×20 mL). The combined organic layers were washed with brine (20 mL), dried over anhydrous Na₂SO₄, and filtered. The filtrate was concentrated under reduced pressure to afford 4-[4-[tert-butoxycarbonyl(cyclopropyl)amino]-1-piperidyl]-2-[[tert-butyl(dimethyl)silyl]oxymethyl]-pyrazolo[1,5-a]pyridine-7-carboxylic acid (232 mg, 425.9 μmol, 72% yield) as a solid. LCMS (ES, m/z): 545 [M+H]⁺.

### Synthesis of B154

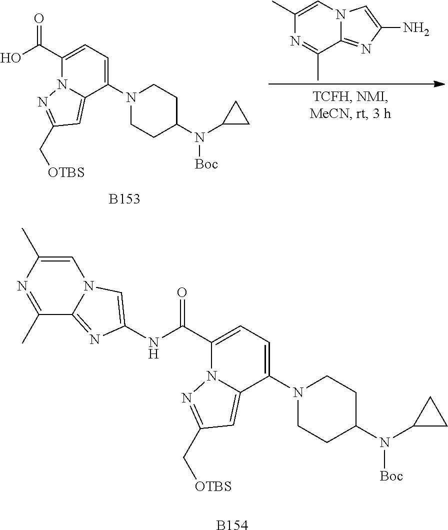 [SMILES: "B153" ; "B154" ; CC(C)(C)OC(=O)N(C1CC1)C1CCN(c2ccc(C(=O)O)n3nc(CO[Si](C)(C)C(C)(C)C)cc23)CC1 ; Cc1cn2cc(NC(=O)c3ccc(N4CCC(N(C(=O)OC(C)(C)C)C5CC5)CC4)c4cc(CO[Si](C)(C)C(C)(C)C)nn34)nc2c(C)n1 ; Cc1cn2cc(N)nc2c(C)n1]

To a stirred mixture of 4-[4-[tert-butoxycarbonyl(cyclopropyl)amino]-1-piperidyl]-2-[[tert-butyl(dimethyl)silyl]oxymethyl]pyrazolo[1,5-a]pyridine-7-carboxylic acid (210 mg, 385.5 μmol) and 6,8-dimethylimidazo[1,2-a]pyrazin-2-amine (75 mg, 462.6 μmol) in MeCN (6 mL) were added NMI (126 mg, 1.54 mmol) and TCFH (162 mg, 578.2 μmol), and the reaction was stirred for 3 h at room temperature. The resulting mixture was diluted with water (10 mL) and MeCN (10 mL). The precipitated solids were collected by filtration and washed with water (2×10 mL). The resulting solid was dried under infrared light to afford tert-butyl N-[1-[2-[[tert-butyl(dimethyl)silyl]oxymethyl]-7-[(6,8-dimethylimidazo[1,2-a]pyrazin-2-yl)-carbamoyl]-pyrazolo[1,5-a]pyridin-4-yl]-4-piperidyl]-N-cyclopropyl-carbamate (241 mg, 349.8 μmol, 91% yield) as a solid. LCMS (ES, m/z): 689 [M+H]⁺.

### Synthesis of Compound 616

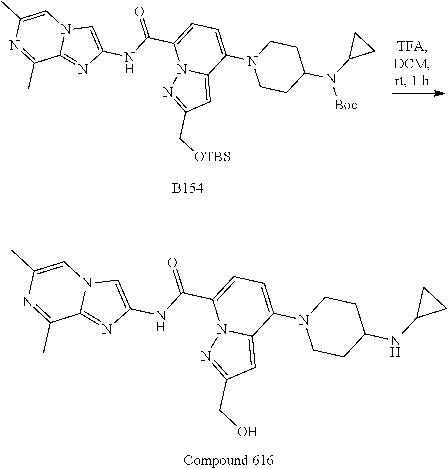 [SMILES: "B154" ; Cc1cn2cc(NC(=O)c3ccc(N4CCC(N(C(=O)OC(C)(C)C)C5CC5)CC4)c4cc(CO[Si](C)(C)C(C)(C)C)nn34)nc2c(C)n1 ; Cc1cn2cc(NC(=O)c3ccc(N4CCC(NC5CC5)CC4)c4cc(CO)nn34)nc2c(C)n1]

A solution of tert-butyl N-[1-[2-[[tert-butyl(dimethyl)silyl]oxymethyl]-7-[(6,8-dimethylimidazo[1,2-a]pyrazin-2-yl)carbamoyl]pyrazolo[1,5-a]pyridin-4-yl]-4-piperidyl]-N-cyclopropyl-carbamate (150 mg, 217.7 μmol) in DCM (2 mL) was treated with TFA (0.5 mL), and the reaction was stirred for 1 h at room temperature under N₂. The resulting mixture was concentrated under reduced pressure and diluted with water (10 mL). The residue was basified to pH 8 with saturated NaHCO₃ (aq.). The precipitated solids were collected by filtration and washed with water (2 mL) and MeOH (1 mL). The resulting solid was dried under infrared light, to afford 4-[4-(cyclopropylamino)-1-piperidyl]-N-(6,8-dimethylimidazo[1,2-a]pyrazin-2-yl)-2-(hydroxymethyl) pyrazolo[1,5-a]-pyridine-7-carboxamide (Compound 616, 58.0 mg, 168.6 μmol, 77% yield) as a solid. LCMS (ES, m/z): 475 [M+H]⁺. ¹H NMR (400 MHz, DMSO-d₆) δ 12.98 (s, 1H), 8.36-8.35 (m, 2H), 7.87 (d, J=8.1 Hz, 1H), 7.05-6.54 (m, 2H), 5.51 (t, J=5.8 Hz, 1H), 4.80 (d, J=5.3 Hz, 2H), 3.75 (d, J=12.1 Hz, 2H), 2.98 (t, J=11.6 Hz, 2H), 2.78 (s, 1H), 2.71 (s, 3H), 2.38 (s, 3H), 2.28 (s, 1H), 2.18-2.10 (m, 1H), 2.03 (d, J=12.6 Hz, 2H), 1.52 (q, J=11.2 Hz, 2H), 0.40 (dt, J=6.2, 3.0 Hz, 2H), 0.24 (p, J=4.0 Hz, 2H).

An analogous method was followed to obtain the following compounds.

| Compound | Stating Material | Characterization |
|---|---|---|
| 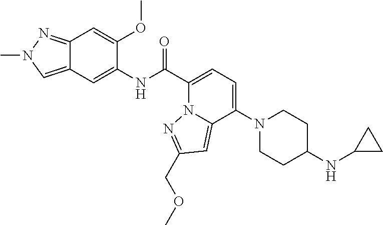 [SMILES: COCc1cc2c(N3CCC(NC4CC4)CC3)ccc(C(=O)Nc3cc4cn(C)nc4cc3OC)n2n1] Compound 621 | 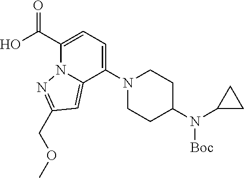 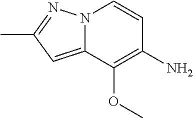 | The residue was purified by reversed-phase flash chromatography (Condition 3, Gradient 25) to afford 4-[4- (cyclopropylamino)-1- piperidyl]-2- (methoxymethyl)-N-(6- methoxy-2-methyl-indazol-5- yl)-pyrazolo[1,5-a]-pyridine- 7-carboxamide (Compound 621, 0.05 g, 99.3 μmol, 40% yield) as a solid. LCMS (ES, m/z): 504 [M + H]⁺. ¹H NMR (400 MHz, DMSO-d₆) δ 13.08 (s, 1H), 8.83 (s, 1H), 8.21 (s, 1H), 7.85 (d, J = 8.1 Hz, 1H), 7.08 (s, 1H), 6.82 (s, 1H), 6.76 (d, J = 8.2 Hz, 1H), 4.73 (s, 2H), 4.07 (s, 3H), 4.03 (s, 3H), |
| | | 3.70 (d, J = 12.2 Hz, 2H), 3.39 |
| | | (s, 3H), 2.93 (t, J = 11.5 Hz, |
| | | 2H), 2.74 (t, J = 7.0 Hz, 1H), |
| | | 2.24 (s, 1H), 2.10 (tt, J = 6.7, |
| | | 3.6 Hz, 1H), 2.00 (d, J = 12.7 |
| | | Hz, 2H), 1.50 (q, J = 10.0 Hz, |
| | | 2H), 0.37 (dt, J = 6.2, 3.0 Hz, |
| | | 2H), 0.29-0.17 (m, 2H). |

### Example 166: Synthesis of Compound 617

### Synthesis of B155

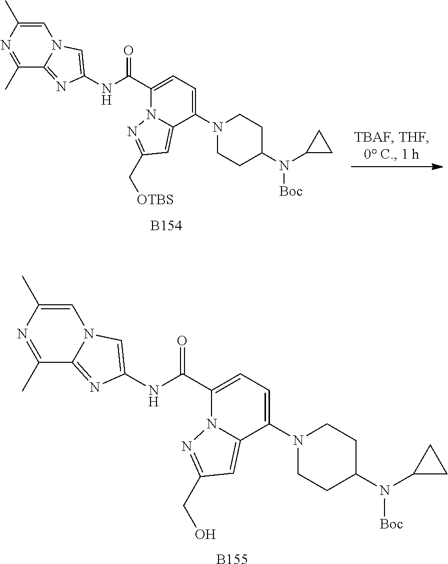 [SMILES: "B154" ; "B155" ; Cc1cn2cc(NC(=O)c3ccc(N4CCC(N(C(=O)OC(C)(C)C)C5CC5)CC4)c4cc(CO[Si](C)(C)C(C)(C)C)nn34)nc2c(C)n1 ; Cc1cn2cc(NC(=O)c3ccc(N4CCC(N(C(=O)OC(C)(C)C)C5CC5)CC4)c4cc(CO)nn34)nc2c(C)n1]

A solution of tert-butyl N-[1-[2-[[tert-butyl(dimethyl)silyl]oxymethyl]-7-[(6,8-dimethyl-imidazo[1,2-a]pyrazin-2-yl)carbamoyl]pyrazolo[1,5-a]pyridin-4-yl]-4-piperidyl]-N-cyclopropyl-carbamate (95 mg, 137.9 μmol) in THF (2 mL) was treated with TBAF (77 mg, 275.8 μmol) at 0° C., and the reaction was stirred for 1 h at 0° C. The resulting mixture was diluted with water (10 mL) and extracted with EtOAc (2×10 mL). The combined organic layers were washed with brine (10 mL), dried over anhydrous Na₂SO₄, and filtered. The filtrate was concentrated under reduced pressure to afford tert-butyl N-cyclopropyl-N-[1-[7-[(6,8-dimethylimidazo[1,2-a]-pyrazin-2-yl)carbamoyl]-2-(hydroxymethyl)-pyrazolo[1,5-a]pyridin-4-yl]-4-piperidyl]carbamate (75 mg, 130.5 μmol, 95% yield) as a solid. LCMS (ES, m/z): 575 [M+H]⁺.

### Synthesis of B156

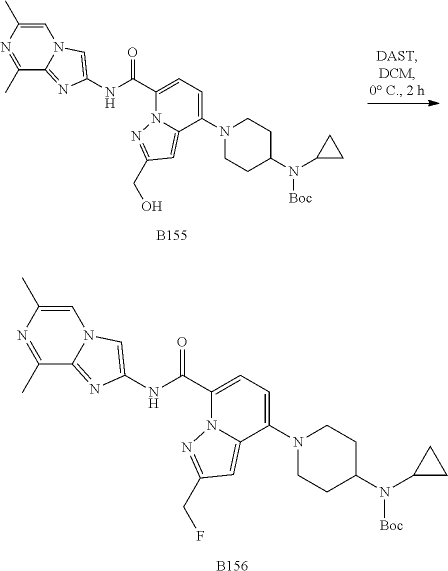 [SMILES: "B155" ; "B156" ; Cc1cn2cc(NC(=O)c3ccc(N4CCC(N(C(=O)OC(C)(C)C)C5CC5)CC4)c4cc(CO)nn34)nc2c(C)n1 ; Cc1cn2cc(NC(=O)c3ccc(N4CCC(N(C(=O)OC(C)(C)C)C5CC5)CC4)c4cc(CF)nn34)nc2c(C)n1]

To a stirred solution of tert-butyl N-cyclopropyl-N-[1-[7-[(6,8-dimethylimidazo[1,2-a]-pyrazin-2-yl)carbamoyl]-2-(hydroxymethyl) pyrazolo[1,5-a]pyridin-4-yl]-4-piperidyl]-carbamate (60 mg, 104.4 μmol) in DCM (2 mL) was added DAST (67 mg, 417.6 μmol) dropwise at 0° C. under N₂ atmosphere. The resulting mixture was stirred for 2 h at 0° C. The resulting mixture was quenched with NaHCO₃ aqueous (10 mL) at 0° C. The resulting mixture was extracted with DCM (3×20 mL). The combined organic layers were washed with brine (20 mL), dried over anhydrous Na₂SO₄, and filtered. The filtrate was concentrated under reduced pressure and purified by silica gel column chromatography (67% EtOAc in PE) to afford tert-butyl N-cyclopropyl-N-[1-[7-[(6,8-dimethylimidazo[1,2-a]pyrazin-2-yl)carbamoyl]-2-(fluoromethyl)-pyrazolo[1,5-a]-pyridin-4-yl]-4-piperidyl]carbamate (40 mg, 69.4 μmol, 66% yield) as a solid. LCMS (ES, m/z): 577 [M+H]⁺.

### Synthesis of Compound 617

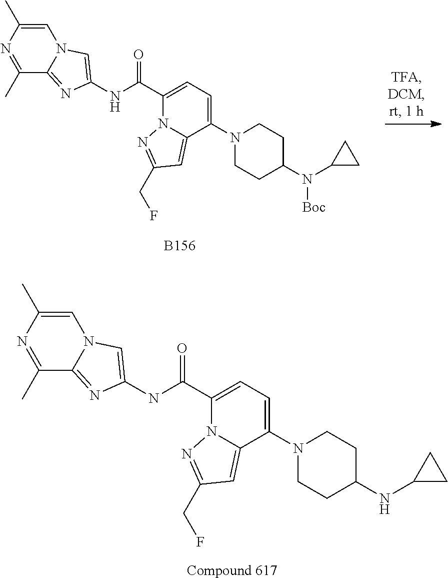

To a stirred mixture of tert-butyl N-cyclopropyl-N-[1-[7-[(6,8-dimethylimidazo[1,2-a]-pyrazin-2-yl)carbamoyl]-2-(fluoromethyl) pyrazolo[1,5-a]pyridin-4-yl]-4-piperidyl]carbamate (35 mg, 60.69 μmol) in DCM (1 mL) was added TFA (0.25 mL) dropwise, and the reaction was stirred for 2 h at room temperature. The resulting mixture was concentrated under reduced pressure and purified by Prep-HPLC (Condition 5, Gradient 24) to afford 4-[4-(cyclopropylamino)-1-piperidyl]-N-(6,8-dimethylimidazo[1,2-a]pyrazin-2-yl)-2-(fluoromethyl)-pyrazolo[1,5-a]pyridine-7-carboxamide (Compound 617, 11 mg, 22.9 μmol, 38% yield) as a solid. LCMS (ES, m/z): 477 [M+H]⁺. ¹H NMR (400 MHz, DMSO-d₆) δ 12.74 (s, 1H), 8.37 (s, 1H), 8.34 (s, 1H), 7.94 (d, J=8.2 Hz, 1H), 7.09 (d, J=2.1 Hz, 1H), 6.83 (d, J=8.3 Hz, 1H), 5.81 (s, 1H), 5.69 (s, 1H), 3.76 (d, J=12.4 Hz, 2H), 3.01 (t, J=11.5 Hz, 2H), 2.84-2.75 (m, 1H), 2.71 (s, 3H), 2.39 (d, J=1.0 Hz, 3H), 2.13 (tt, J=6.7, 3.6 Hz, 1H), 2.03 (d, J=12.7 Hz, 2H), 1.53 (q, J=11.2, 10.5 Hz, 2H), 0.41 (td, J=6.3, 4.0 Hz, 2H), 0.31-0.18 (m, 2H).

An analogous method was followed to obtain the following compounds.

| Compound | Starting Material | Characterization |
|---|---|---|
| 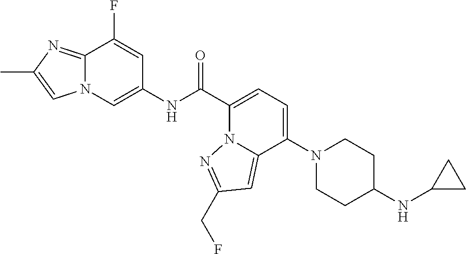 [SMILES: Cc1cn2cc(NC(=O)c3ccc(N4CCC(NC5CC5)CC4)c4cc(CF)nn34)cc(F)c2n1] | 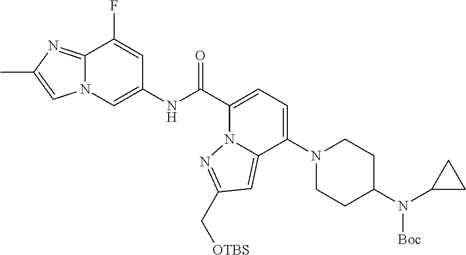 [SMILES: Cc1cn2cc(NC(=O)c3ccc(N4CCC(N(C(=O)OC(C)(C)C)C5CC5)CC4)c4cc(CO[Si](C)(C)C(C)(C)C)nn34)cc(F)c2n1] | Modification: Step 1 was ran for 2 h at room temperature. Step 2 was ran at room temperature for 1 h. Step 3 was ran using HCl in dioxane and DCM for 1 h at room temperature. The residue was purified by reversed-phase flash chromatography (Condition 3, Gradient 15) to afford 4- [4-(cyclopropylamino)-1- piperidyl]-2- (fluoromethyl)-N-(8-fluoro- 2-methyl-imidazo[1,2-a]- pyridin-6-y1)pyrazolo[1,5- |
| Compound 623 | | a]pyridine-7-carboxamide |
| | | (Compound 623, 0.02 g, |
| | | 41.7 μmol, 19% yield) as a |
| | | solid. LCMS (ES, m/z): 480 |
| | | [M + H]⁺. ¹H NMR (300 |
| | | MHz, DMSO-d₆) δ 12.31 (s, |
| | | 1H), 9.22 (d, J = 1.7 Hz, |
| | | 1H), 7.93 (d, J = 3.1 Hz, |
| | | 1H), 7.83 (d, J = 8.1 Hz, |
| | | 1H), 7.43 (d, J = 12.4 Hz, |
| | | 1H), 7.04 (d, J = 2.0 Hz, |
| | | 1H), 6.80 (d, J = 8.3 Hz, |
| | | 1H), 5.86 (s, 1H), 5.70 (s, |
| | | 1H), 3.74 (d, J = 12.5 Hz, |
| | | 2H), 2.99 (t, J = 11.3 Hz, |
| | | 2H), 2.78 (s,1H), 2.36 (s, |
| | | 3H), 2.29 (s, 1H), 2.18-2.07 |
| | | (m, 1H), 2.02 (d, J = 12.7 |
| | | Hz, 2H), 1.52 (q, J = 11.0 |
| | | Hz, 2H), 0.40 (dt, J = 6.2, |
| | | 3.0 Hz, 2H), 0.24 (p, J = 3.9 |
| | | Hz, 2H). |
| 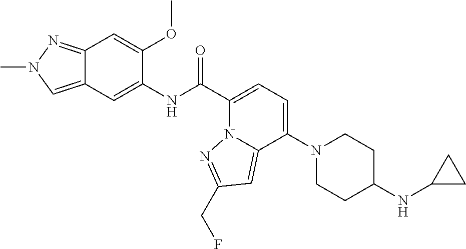 [SMILES: COc1cc2nn(C)cc2cc1NC(=O)c1ccc(N2CCC(NC3CC3)CC2)c2cc(CF)nn12] | 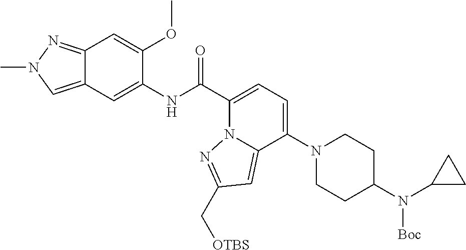 [SMILES: COc1cc2nn(C)cc2cc1NC(=O)c1ccc(N2CCC(N(C(=O)OC(C)(C)C)C3CC3)CC2)c2cc(CO[Si](C)(C)C(C)(C)C)nn12] | Modification: Step 1 was ran for 2 h at room temperature. Step 2 was ran for 2 h. Step 3 was ran using HCl in dioxane and DCM for 1 h at room temperature. The residue was purified by reversed-phase flash chromatography (Condition 5, Gradient 8) to afford 4-[4- (cyclopropylamino)-1- piperidyl]-2- (fluoromethyl)-N-(6- methoxy-2-methyl-indazol- 5-yl)pyrazolo[1,5-a]- |
| Compound 625 | | pyridine-7-carboxamide |
| | | (Compound 625, 0.004 g, |
| | | 8.14 μmol, 5% yield) as a |
| | | solid. LCMS (ES, m/z): 492 |
| | | [M + H]⁺. ¹H NMR (400 |
| | | MHz, Methanol-d₄) δ 8.86 |
| | | (s, 1H), 8.09 (s, 1H), 7.98 (d, |
| | | J = 8.1 Hz, 1H), 7.06 (s, |
| | | 1H), 6.93 (d, J = 2.0 Hz, |
| | | 1H), 6.80 (d, J = 8.2 Hz, |
| | | 1H), 5.78 (s, 1H), 5.66 (s, |
| | | 1H), 4.17 (s, 3H), 4.12 (s, |
| | | 3H), 3.89 (d, J = 12.6 Hz, |
| | | 2H), 3.03-2.84 (m, 2H), 2.27 |
| | | (dq, J = 6.9, 3.5 Hz, 1H), |
| | | 2.11 (s, 1H), 2.19 (d, J = 12.3 |
| | | Hz, 2H), 1.68 (qd, J = 12.1, |
| | | 3.8 Hz, 2H), 0.56 (td, J = |
| | | 6.7, 4.6 Hz, 2H), 0.48-0.40 |
| | | (m, 2H). |

### Example 167: Synthesis of C160

### Synthesis of C158

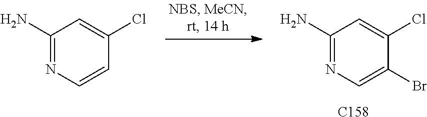 [SMILES: Nc1cc(Cl)ccn1 ; Nc1cc(Cl)c(Br)cn1 ; "C158"]

To a solution of 4-chloropyridin-2-amine (10 g, 77.79 mmol) in MeCN (100 mL) was added NBS (13.84 g, 77.79 mmol). The reaction was stirred for 14 h at rt. The mixture was diluted with water (300 mL) and extracted with EtOAc (2×300 mL). The combined organic layer was washed with water (2×300 mL) and brine (300 mL), dried by anhydrous Na₂SO₄, and filtered. The filtrate was concentrated under reduced pressure and purified by silica gel column chromatography (17% EtOAc in PE) to afford 5-bromo-4-chloro-pyridin-2-amine (9.5 g, 45.8 mmol, 59% yield) as a solid. LCMS (ES, m/z): 207 [M+H]⁺.

### Synthesis of C159

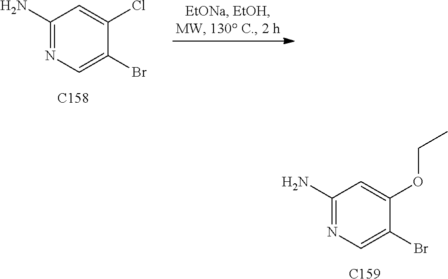

To a solution of 5-bromo-4-chloro-pyridin-2-amine (3 g, 14.46 mmol) in EtOH (30 mL) was added EtONa (1.97 g, 28.92 mmol, 2.27 mL). The reaction was stirred for 2 h at MW 130° C. The mixture diluted with water (100 mL) and extracted with EtOAc (2×100 mL). The combined organic layer was washed with water (2×100 mL) and brine (100 mL), dried by anhydrous Na₂SO₄, and filtered. The filtrate was concentrated under reduced pressure and purified by silica gel column chromatography (83% EtOAc in PE) to afford 5-bromo-4-ethoxy-pyridin-2-amine (2 g, 9.21 mmol, 64% yield) as a solid. LCMS (ES, m/z): 217 [M+H]⁺.

### Synthesis of C160

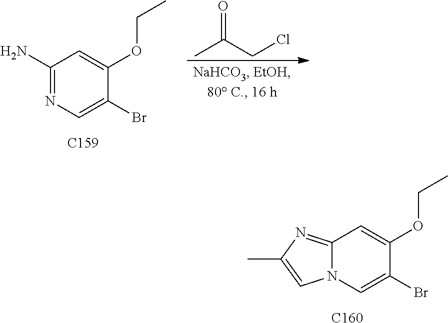

To a solution of 5-bromo-4-ethoxy-pyridin-2-amine (1 g, 4.61 mmol) in EtOH (20 mL) were added 1-chloropropan-2-one (511 mg, 5.53 mmol) and NaHCO₃ (774 mg, 9.22 mmol), and the reaction was stirred for 16 h at 80° C. The resulting mixture was filtered, concentrated under reduced pressure, and purified by silica gel column chromatography (25% EtOAc in PE) to afford 6-bromo-7-ethoxy-2-methyl-imidazo[1,2-a]pyridine (0.4 g, 1.57 mmol, 34% yield) as a solid. LCMS (ES, m/z): 255 [M+H]⁺.

### Example 168: Synthesis of C164

### Synthesis of C161

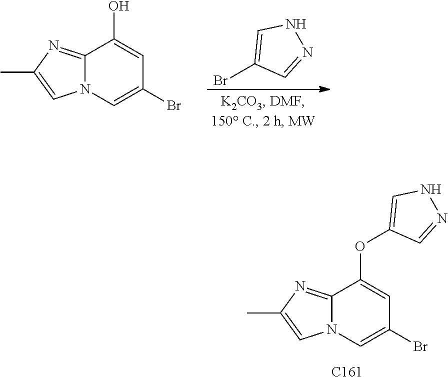 [SMILES: Cc1cn2cc(Br)cc(O)c2n1 ; Brc1cn[nH]c1 ; Cc1cn2cc(Br)cc(Oc3cn[nH]c3)c2n1 ; "C161"]

To a solution of 6-bromo-2-methyl-imidazo[1,2-a]pyridin-8-ol (1.5 g, 6.61 mmol) in DMF (15 mL) were added 4-bromo-1H-pyrazole (1.94 g, 13.21 mmol) and K₂CO₃ (1.83 g, 13.21 mmol). The mixture was stirred for 2 h at MW 140° C. The reaction was filtered and the filtrate was purified by Prep-HPLC (Condition 17, Gradient 2) to afford 6-bromo-2-methyl-8-(1H-pyrazol-4-yloxy)imidazo[1,2-a]pyridine (600 mg, 2.05 mmol, 31% yield) as a solid. LCMS (ES, m/z): 293 [M+H]⁺.

### Synthesis of C162

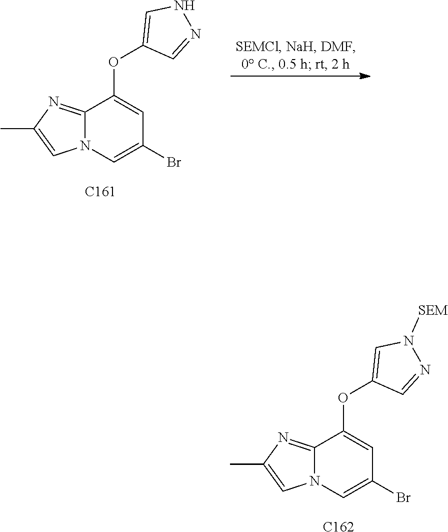 [SMILES: Cc1cn2cc(Br)cc(Oc3cn[nH]c3)c2n1 ; "C161" ; Cc1cn2cc(Br)cc(Oc3cnn(COCC[Si](C)(C)C)c3)c2n1 ; "C162"]

To a solution of 6-bromo-2-methyl-8-(1H-pyrazol-4-yloxy)imidazo[1,2-a]pyridine (600 mg, 2.05 mmol) in DMF (6 mL) was added NaH (60% dispersion in oil) (123 mg, 3.07 mmol, 60% purity) at 0° C. under N₂. The mixture was stirred for 0.5 h. Then SEMCl (410 mg, 2.46 mmol) was added at 0° C. under N₂. The mixture was stirred for 2 h at rt. The reaction was quenched with ice-water (30 mL) and extracted with EtOAc (2×30 mL). The combined organic layers were washed with water (2×50 mL), dried over anhydrous Na₂SO₄, and filtered. The filtrate was concentrated under reduced pressure and purified by silica gel column chromatography (25% EtOAc in DCM) to afford to afford 2-[[4-(6-bromo-2-methyl-imidazo[1,2-a]pyridin-8-yl)oxypyrazol-1-yl]methoxy]ethyl-trimethyl-silane (360 mg, 850.3 μmol, 42% yield) as an oil. LCMS (ES, m/z): 423 [M+H]⁺.

### Synthesis of C163

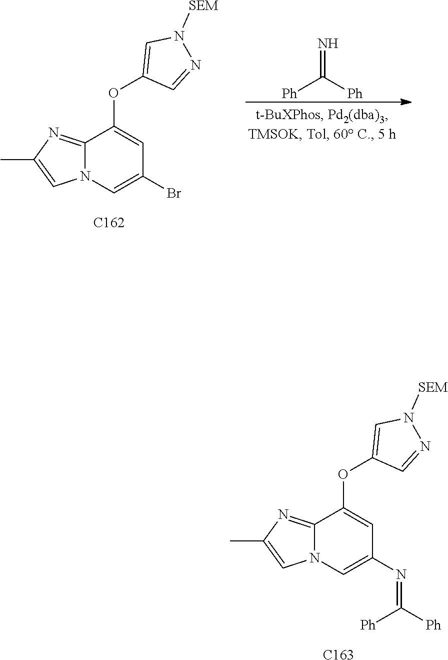 [SMILES: Cc1cn2cc(Br)cc(Oc3cnn(COCC[Si](C)(C)C)c3)c2n1 ; N=C(c1ccccc1)c1ccccc1 ; "C162" ; Cc1cn2cc(N=C(c3ccccc3)c3ccccc3)cc(Oc3cnn(COCC[Si](C)(C)C)c3)c2n1 ; "C163"]

To a stirred mixture of 2-[[4-(6-bromo-2-methyl-imidazo[1,2-a]pyridin-8-yl)oxypyrazol-1-yl]methoxy]ethyl-trimethyl-silane (65 mg, 153.5 μmol) in toluene (1 mL) were added diphenylmethanimine (33 mg, 184.2 μmol, 30.92 μL), TMSOK (39 mg, 307.1 μmol), t-BuXPhos (21 mg, 30.7 μmol) and Pd₂(dba)₃ (14 mg, 15.35 μmol) at rt under N₂. The mixture was stirred for 5 h at 60° C. The reaction was concentrated under reduced pressure and purified by silica gel column chromatography (33% EtOAc in DCM) to afford to afford N-[2-methyl-8-[1-(2-trimethylsilylethoxymethyl)pyrazol-4-yl]oxy-imidazo[1,2-a]pyridin-6-yl]-1,1-diphenyl-methanimine (80 mg, 152.8 μmol, 99.5% yield) as an oil. LCMS (ES, m/z): 524 [M+H]⁺.

### Synthesis of C164

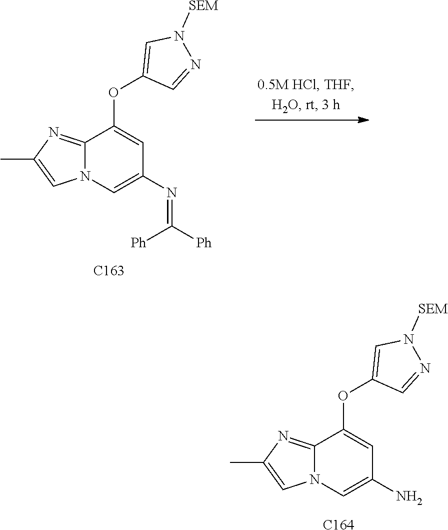 [SMILES: Cc1cn2cc(N=C(c3ccccc3)c3ccccc3)cc(Oc3cnn(COCC[Si](C)(C)C)c3)c2n1 ; "C163" ; Cc1cn2cc(N)cc(Oc3cnn(COCC[Si](C)(C)C)c3)c2n1 ; "C164"]

To a solution of N-[2-methyl-8-[1-(2-trimethylsilylethoxymethyl)pyrazol-4-yl]oxy-imidazo[1,2-a]pyridin-6-yl]-1,1-diphenyl-methanimine (80 mg, 152.8 μmol) in water (0.8 mL) and THF (0.8 mL), was added HCl (0.5 M) (1.6 mL) and the reaction was stirred for 3 h at rt. The resulting mixture was basified to pH 8 with saturated NaHCO₃ (aq.) and extracted with DCM (4×5 mL). The combined organic layers were washed with water (10 mL) and dried over anhydrous Na₂SO₄, and filtered. The filtrate was concentrated under reduced pressure, to afford 2-methyl-8-[1-(2-trimethylsilylethoxymethyl)pyrazol-4-yl]oxy-imidazo[1,2-a]pyridin-6-amine (50 mg, 139.1 μmol, 91% yield) as an oil. LCMS (ES, m/z): 360 [M+H]⁺.

### Example 169: Synthesis of C166

### Synthesis of C165

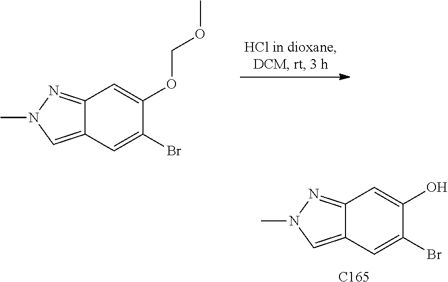

To a stirred mixture of 5-bromo-6-(methoxymethoxy)-2-methyl-indazole (200 mg, 737.7 μmol) in DCM (2 mL) was added HCl (4.0 M in 1,4-dioxane, 2 mL), and the reaction was stirred for 3 h at room temperature. The resulting mixture was concentrated under reduced pressure to afford 5-bromo-2-methyl-indazol-6-ol (160 mg, 704.7 μmol, 96% yield) as a solid. LCMS (ES, m/z): 227 [M+H]⁺.

### Synthesis of C166

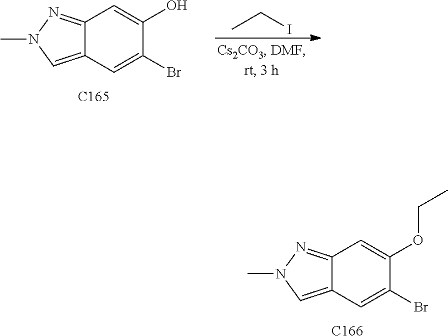

To a stirred solution of 5-bromo-2-methyl-indazol-6-ol (90 mg, 396.4 μmol) and Cs₂CO₃ (258 mg, 792.8 μmol) in DMF (1 mL) was added iodoethane (92 mg, 594.6 μmol), and the reaction was stirred for 3 h at rt. The resulting mixture was diluted with water (10 mL) and extracted with EtOAc (3×8 mL). The combined organic layers were washed with water (2×10 mL) and brine (10 mL), dried over anhydrous Na₂SO₄, and filtered. The filtrate was concentrated under reduced pressure and purified by silica gel column chromatography (50% EtOAc in PE) to afford 5-bromo-6-ethoxy-2-methyl-indazole (89 mg, 348.9 μmol, 88% yield) as a solid. LCMS (ES, m/z): 255 [M+H]⁺.

### Example 170: Synthesis of C169

### Synthesis of C167

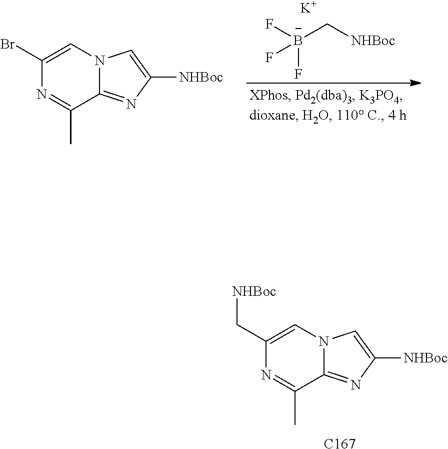 [SMILES: [K+] ; Cc1nc(Br)cn2cc(NC(=O)OC(C)(C)C)nc12 ; CC(C)(C)OC(=O)NC[B-](F)(F)F ; Cc1nc(CNC(=O)OC(C)(C)C)cn2cc(NC(=O)OC(C)(C)C)nc12 ; "C167"]

To a stirred mixture of tert-butyl N-(6-bromo-8-methyl-imidazo[1,2-a]pyrazin-2-yl)carbamate (200 mg, 611.3 μmol) and (tert-butoxycarbonylamino)methyl-trifluoro-boranuide (181.5 mg, 916.9 μmol) in Dioxane (4 mL) and H₂O (0.8 mL) were added K₃PO₄ (389.2 mg, 1.83 mmol), XPhos (58.2 mg, 122.3 μmol) and Pd₂(dba)₃ (55.9 mg, 61.13 μmol) at room temperature under N₂. The resulting mixture was stirred for 4 h at 110° C. under N₂. The resulting mixture was concentrated under reduced pressure and purified by silica gel column chromatography (25% EtOAc in PE) to afford tert-butyl N-[[2-(tert-butoxycarbonylamino)-8-methyl-imidazo[1,2-a]pyrazin-6-yl]methyl]carbamate (200 mg, 529.9 μmol, 87% yield) as a solid. LCMS (ES, m/z): 378 [M+H]⁺.

### Synthesis of C168

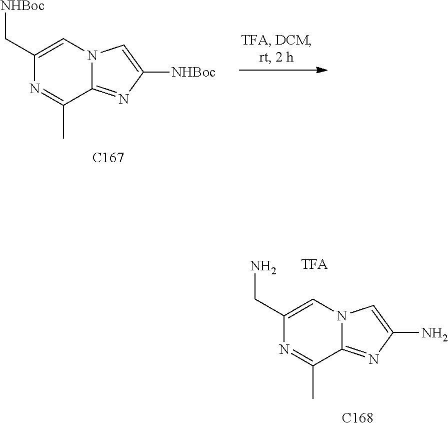 [SMILES: Cc1nc(CNC(=O)OC(C)(C)C)cn2cc(NC(=O)OC(C)(C)C)nc12 ; "C167" ; Cc1nc(CN)cn2cc(N)nc12 ; "C168"]

To a stirred solution of tert-butyl N-[[2-(tert-butoxycarbonylamino)-8-methyl-imidazo[1,2-a]pyrazin-6-yl]methyl]carbamate (200 mg, 529.9 μmol) in DCM (4 mL) was added TFA (2 mL), and the reaction was stirred for 2 h at room temperature. The resulting mixture was concentrated under reduced pressure to afford 6-(aminomethyl)-8-methyl-imidazo[1,2-a]pyrazin-2-amine 2,2,2-trifluoroacetate (80 mg, 451.5 μmol, 85% yield) as a solid. LCMS (ES, m/z): 178 [M+H]⁺.

### Synthesis of C169

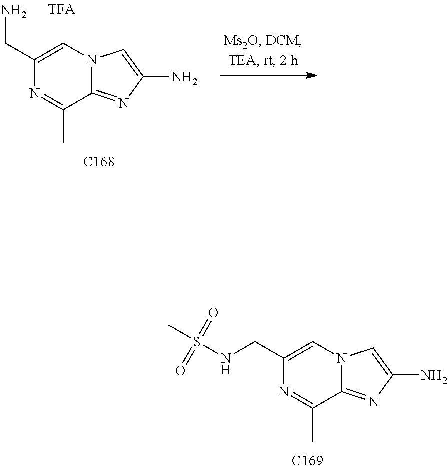 [SMILES: Cc1nc(CN)cn2cc(N)nc12 ; "C168" ; Cc1nc(CNS(C)(=O)=O)cn2cc(N)nc12 ; "C169"]

To a stirred mixture of 6-(aminomethyl)-8-methyl-imidazo[1,2-a]pyrazin-2-amine 2,2,2-trifluoroacetate (80 mg, 451.5 μmol) and TEA (228.4 mg, 2.26 mmol) in DCM (2 mL) were added Ms₂O (78.5 mg, 451.5 μmol) at 0° C. The resulting mixture was stirred for 2 h at room temperature. The resulting mixture was concentrated under reduced pressure and purified by silica gel column chromatography (33% EtOAc in PE) to afford N-[(2-amino-8-methyl-imidazo[1,2-a]pyrazin-6-yl)methyl]methanesulfonamide (90 mg, 352.5 μmol, 78% yield) as a solid. LCMS (ES, m/z): 256 [M+H]⁺.

### Example 171: Synthesis of C171

### Synthesis of C170

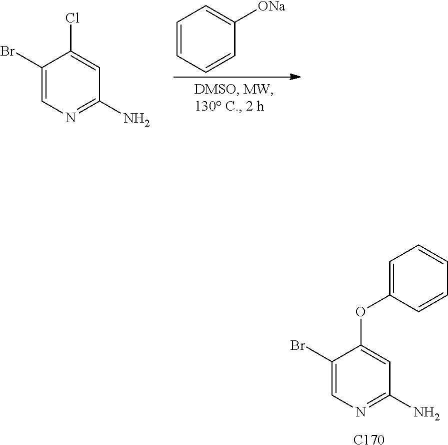

To a mixture of 5-bromo-4-chloro-pyridin-2-amine (3 g, 14.46 mmol) in DMSO (50 mL) was added phenoxysodium (3.36 g, 28.92 mmol), and the reaction was treated with microwave radiation for 2 h at 130° C. The resulting mixture was diluted with water (100 mL) and extracted with EtOAc (3×50 mL). The combined organic layers were washed with brine (2×100 mL), dried over anhydrous Na₂SO₄, and filtered. The filtrate was concentrated under reduced pressure and purified by silica gel column chromatography (50% EtOAc in PE) to afford 5-bromo-4-phenoxy-pyridin-2-amine (2.3 g, 8.68 mmol, 60% yield) as a solid. LCMS (ES, m/z): 265 [M+H]⁺.

### Synthesis of C171

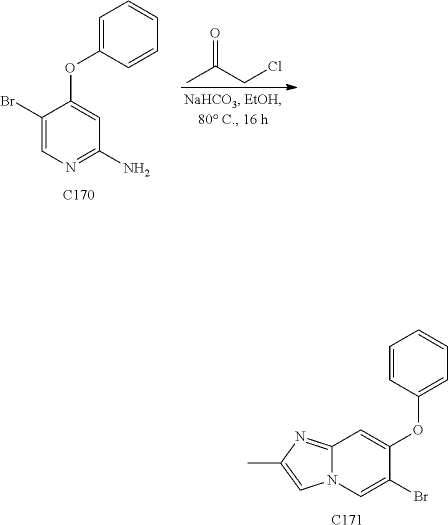

To a stirred mixture of NaHCO₃ (475.3 mg, 5.66 mmol) and 5-bromo-4-phenoxy-pyridin-2-amine (0.5 g, 1.89 mmol) in EtOH (10 mL) were added 1-chloropropan-2-one (209.4 mg, 2.26 mmol) in portions at room temperature under N₂. The resulting mixture was stirred for 16 h at 80° C. The resulting mixture was concentrated under reduced pressure and purified by silica gel column chromatography (50% EtOAc in PE) to afford 6-bromo-2-methyl-7-phenoxy-imidazo-[1,2-a]pyridine (0.2 g, 659.7 μmol, 35% yield) as a solid. LCMS (ES, m/z): 303 [M+H]⁺.

### Example 172: Synthesis of C173

### Synthesis of C172

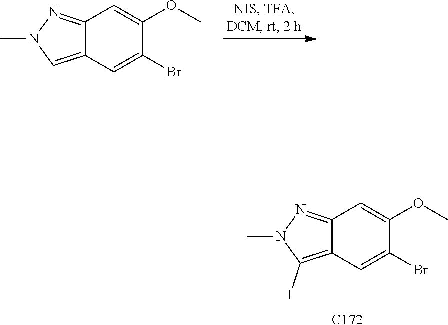

To a solution of 5-bromo-6-methoxy-2-methyl-indazole (1 g, 4.15 mmol) in DCM (10 mL) were added NIS (933 mg, 4.15 mmol) and TFA (473 mg, 4.15 mmol). The mixture was stirred for 2 h at rt. The resulting mixture was diluted with H₂O and adjusted to pH 8 with NaHCO₃. The resulting mixture was extracted with DCM (2×15 mL). The combined organic layers were washed with H₂O (2×20 mL), dried over anhydrous Na₂SO₄, and filtered. The filtrate was concentrated under reduced pressure and purified by silica gel column chromatography (50% EtOAc in PE) to afford 5-bromo-3-iodo-6-methoxy-2-methyl-indazole (340 mg, 926.5 μmol, 22% yield) as an oil. ¹H NMR (400 MHz, DMSO-d₆) δ 7.62 (s, 1H), 7.12 (s, 1H), 4.13 (s, 3H), 3.88 (s, 3H).

### Synthesis of C173

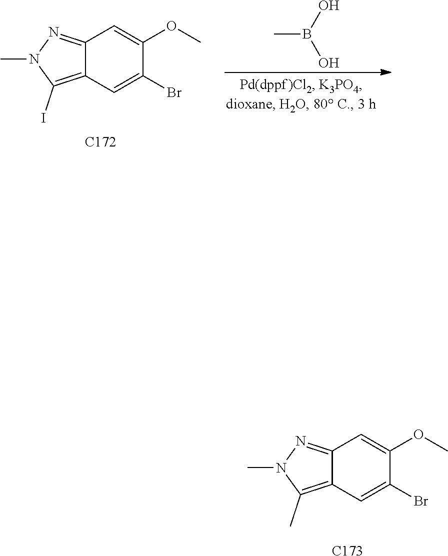

To a stirred mixture of 5-bromo-3-iodo-6-methoxy-2-methyl-indazole (160 mg, 436 μmol) in dioxane (1.6 mL)/H₂O (0.16 mL) were added methylboronic acid (131 mg, 2.18 mmol), K₃PO₄ (278 mg, 1.31 mmol) and Pd(dppf)Cl₂—CH₂Cl₂ (36 mg, 43.6 μmol), and the reaction was stirred for 3 h at 80° C. The reaction was diluted with H₂O (15 mL) and extracted with DCM (2×15 mL). The combined organic layers were washed with H₂O (2×20 mL), dried over anhydrous Na₂SO₄, and filtered. The filtrate was concentrated under reduced pressure and purified by silica gel column chromatography (5% MeOH in DCM) to afford 5-bromo-6-methoxy-2,3-dimethyl-indazole (100 mg, 392 μmol, 90% yield) as a solid. LCMS (ES, m/z): 255 [M+H]⁺.

### Example 173: Synthesis of C175

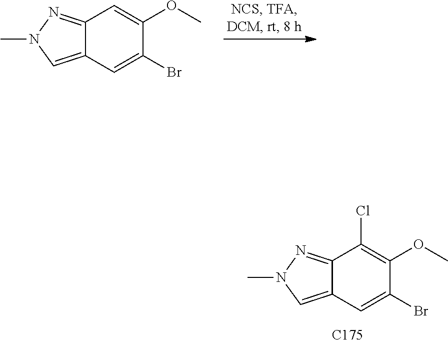

To a solution of 5-bromo-6-methoxy-2-methyl-indazole (0.2 g, 829.6 μmol) in DCM (1 mL) was added TFA (0.4 mL). The reaction was additional added NCS (110 mg, 829.6 μmol) and stirred for 16 h at rt. The resulting mixture was diluted with water (5 mL) and extracted with EtOAc (2×10 mL). The combined organic layers were washed with water (2×20 mL), brine (20 mL), dried over anhydrous Na₂SO₄, and filtered. The filtrate was concentrated under reduced pressure, to afford 5-bromo-7-chloro-6-methoxy-2-methyl-indazole (0.2 g, 725.9 μmol, 88% yield) as an oil. LCMS (ES, m/z): 275 [M+H]⁺.

An analogous method was followed to obtain the following compounds.

| Compound | Starting Material | Characterization |
|---|---|---|
| 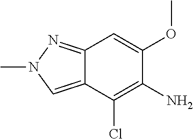 | 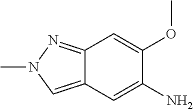 | Modification: The reaction was ran in MeCN for 2 h at room temperature. The residue was purified by silica gel column chromatography (67% EtOAc in PE) to afford 4- chloro-6-methoxy-2-methyl-indazol-5-amine (C209, 70 mg, 330.7 μmol, 59% yield) as a solid. LCMS (ES, m/z): 212 [M + H]⁺. |
| C209 | | |

### Example 174: Synthesis of C178

### Synthesis of C176

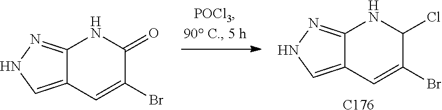 [SMILES: O=c1[nH]c2n[nH]cc2cc1Br ; ClC1Nc2n[nH]cc2C=C1Br ; "C176"]

A solution of 5-bromo-2,7-dihydro-6H-pyrazolo[3,4-b]pyridin-6-one (1 g, 4.69 mmol) in POCl₃ (10 mL) was stirred for 5 h at 90° C. The reaction was quenched with water (100 mL) at 0° C. The resulting mixture was extracted with EtOAc (2×100 mL). The combined organic layers were washed with water (2×100 mL) and brine (100 mL), dried over anhydrous Na₂SO₄, and filtered. The filtrate was concentrated under reduced pressure and purified by silica gel column chromatography (50% EtOAc in PE) to afford 5-bromo-6-chloro-2H-pyrazolo[3,4-b]pyridine (600 mg, 2.6 mmol, 55% yield) as a solid. LCMS (ES, m/z): 232 [M+H]⁻.

### Synthesis of C177

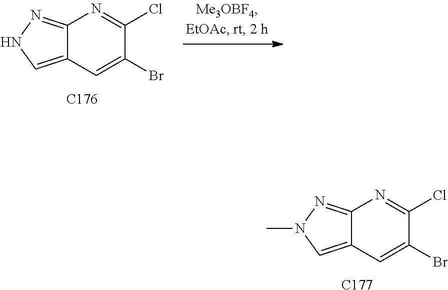

To a stirred mixture of 5-bromo-6-chloro-2H-pyrazolo[3,4-b]pyridine (500 mg, 2.15 mmol) in EtOAc (10 mL) was added Me₃OBF₄ (318.3 mg, 2.15 mmol) at room temperature under. The resulting mixture was stirred for 2 h at rt. The reaction was monitored by LCMS. The reaction was diluted with water (20 mL) and extracted with EtOAc (2×20 mL). The combined organic layers were washed with brine (20 mL), dried over anhydrous Na₂SO₄, and filtered. The filtrate was concentrated under reduced pressure and purified by silica gel column chromatography (50% EtOAc in PE) to afford 5-bromo-6-chloro-2-methyl-pyrazolo[3,4-b]-pyridine (180 mg, 730.3 μmol, 34% yield) as a solid. LCMS (ES, m/z): 246 [M+H]⁺.

### Synthesis of C178

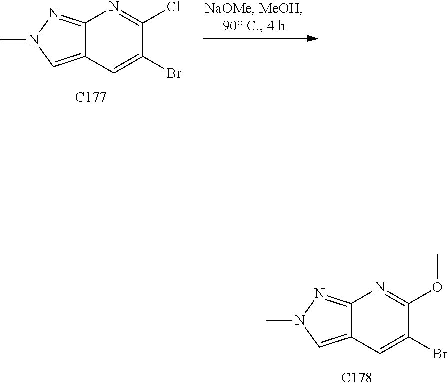

To a stirred mixture of 5-bromo-6-chloro-2-methyl-pyrazolo[3,4-b]pyridine (150 mg, 608.5 μmol) in MeOH (3 mL) was added NaOMe (230.1 mg, 4.26 mmol), and the resulting mixture was stirred for 4 h at 90° C. The reaction was quenched with water (20 mL) at 5° C. The resulting mixture was extracted with EtOAc (2×20 mL). The combined organic layers were washed with brine (20 mL), dried over anhydrous Na₂SO₄, and filtered. The filtrate was concentrated under reduced pressure and purified by silica gel column chromatography (50% EtOAc in PE) to afford 5-bromo-6-methoxy-2-methyl-pyrazolo[3,4-b]pyridine (C178, 120 mg, 495.7 μmol, 81% yield) as a solid. LCMS (ES, m/z): 242 [M+H]⁺.

### Example 175: Synthesis of Compound 631

### Synthesis of B157

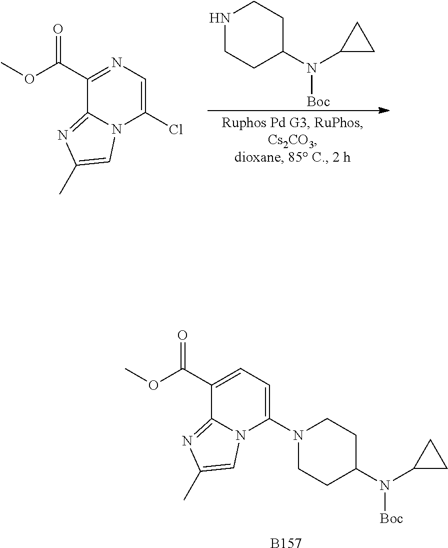 [SMILES: COC(=O)c1ncc(Cl)n2cc(C)nc12 ; CC(C)(C)OC(=O)N(C1CCNCC1)C1CC1 ; "B157" ; COC(=O)c1ccc(N2CCC(N(C(=O)OC(C)(C)C)C3CC3)CC2)n2cc(C)nc12]

To a stirred mixture of methyl 5-chloro-2-methyl-imidazo[1,2-a]pyridine-8-carboxylate (500 mg, 2.23 mmol) in dioxane (5 mL) were added tert-butyl N-cyclopropyl-N-(4-piperidyl)carbamate (641.9 mg, 2.67 mmol), Cs₂CO₃ (1.45 g, 4.45 mmol), Ruphos (207.7 mg, 445.2 μmol) and RuPhos Pd G3 (186.1 mg, 222.6 μmol) at room temperature under N₂. The mixture was stirred for 2 h at 80° C. The reaction was concentrated under reduced pressure and purified by silica gel column chromatography (40% THF in PE) to afford methyl 5-[4-[tert-butoxycarbonyl(cyclopropyl)amino]-1-piperidyl]-2-methyl-imidazo[1,2-a]pyridine-8-carboxylate (B157, 600 mg, 1.4 mmol, 63% yield) as an oil. LCMS (ES, m/z): 429 [M+H]⁺.

### Synthesis of B158

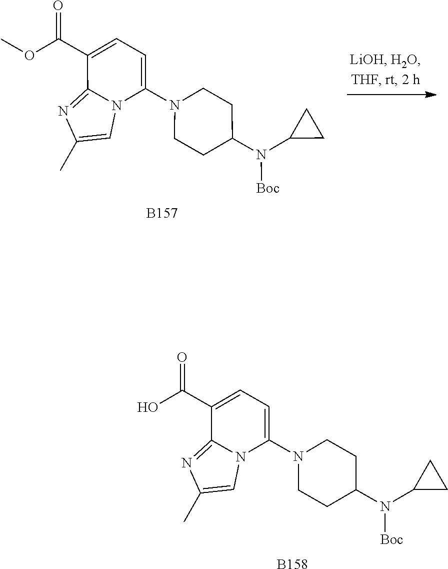 [SMILES: "B157" ; "B158" ; COC(=O)c1ccc(N2CCC(N(C(=O)OC(C)(C)C)C3CC3)CC2)n2cc(C)nc12 ; Cc1cn2c(N3CCC(N(C(=O)OC(C)(C)C)C4CC4)CC3)ccc(C(=O)O)c2n1]

To a solution of methyl 5-[4-[tert-butoxycarbonyl(cyclopropyl)amino]-1-piperidyl]-2-methyl-imidazo[1,2-a]pyridine-8-carboxylate (600 mg, 1.40 mmol) in THF (5 mL) and H₂O (2.5 mL) was added LiOH·H₂O (293.7 mg, 7.00 mmol), and the reaction was stirred for 2 h at rt. The resulting mixture was diluted with H₂O and adjusted to pH 5-6 with 1 N HCl. The resulting mixture was extracted with DCM (2×40 mL). The combined organic layers were washed with H₂O (2×40 mL), dried over anhydrous Na₂SO₄, and filtered. The filtrate was concentrated under reduced pressure to afford 5-[4-[tert-butoxycarbonyl(cyclopropyl)amino]-1-piperidyl]-2-methyl-imidazo[1,2-a]pyridine-8-carboxylic acid (B158, 500 mg, 1.21 mmol, 86% yield) as a solid. LCMS (ES, m/z): 415 [M+H]⁺.

### Synthesis of B159

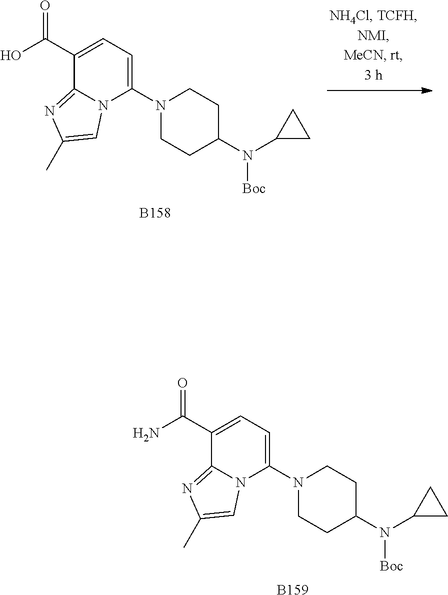 [SMILES: "B158" ; "B159" ; Cc1cn2c(N3CCC(N(C(=O)OC(C)(C)C)C4CC4)CC3)ccc(C(=O)O)c2n1 ; Cc1cn2c(N3CCC(N(C(=O)OC(C)(C)C)C4CC4)CC3)ccc(C(N)=O)c2n1]

To a solution of 5-[4-[tert-butoxycarbonyl(cyclopropyl)amino]-1-piperidyl]-2-methyl-imidazo[1,2-a]pyridine-8-carboxylic acid (480 mg, 1.16 mmol) in MeCN (5 mL) were added NH₄Cl (185.8 mg, 3.47 mmol, 121.5 μL), NMI (380.3 mg, 4.63 mmol) and TCFH (487.3 mg, 1.74 mmol). The mixture was stirred for 3 h at rt. The reaction was diluted with H₂O (15 mL). The precipitated solids were collected by filtration and purified by trituration with MTBE (10 mL) to afford tert-butyl N-[1-(8-carbamoyl-2-methyl-imidazo[1,2-a]pyridin-5-yl)-4-piperidyl]-N-cyclopropyl-carbamate (B159, 380 mg, 918.9 μmol, 79% yield) as a solid. LCMS (ES, m/z): 414 [M+H]⁺.

### Synthesis of B160

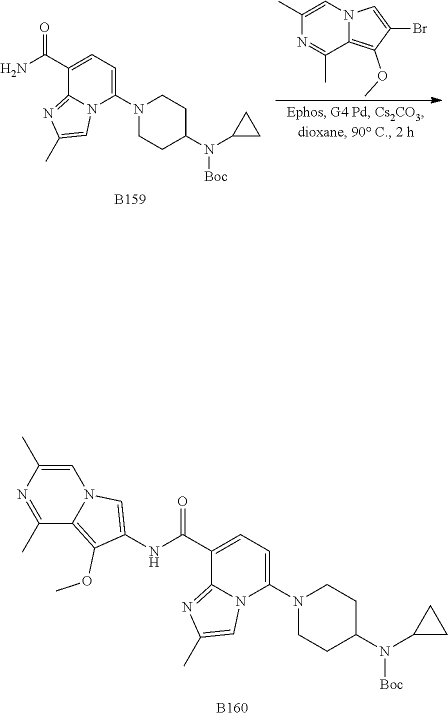 [SMILES: "B159" ; "B160" ; Cc1cn2c(N3CCC(N(C(=O)OC(C)(C)C)C4CC4)CC3)ccc(C(N)=O)c2n1 ; COc1c(NC(=O)c2ccc(N3CCC(N(C(=O)OC(C)(C)C)C4CC4)CC3)n3cc(C)nc23)cn2cc(C)nc(C)c12 ; COc1c(Br)cn2cc(C)nc(C)c12]

To a stirred mixture of 7-bromo-8-methoxy-1,3-dimethyl-pyrrolo[1,2-a]pyrazine (27.7 mg, 108.8 μmol) and tert-butyl N-[1-(8-carbamoyl-2-methyl-imidazo[1,2-a]pyridin-5-yl)-4-piperidyl]-N-cyclopropyl-carbamate (45 mg, 108.8 μmol) in dioxane (2 mL) were added Cs₂CO₃ (70.9 mg, 217.7 μmol) and Ephos G₄ Pd (9.9 mg, 10.9 μmol), and the reaction was stirred for 2 h at 90° C. under N₂. The resulting mixture was concentrated under reduced pressure and purified by silica gel column chromatography (50% EtOAc in PE) to afford tert-butyl N-cyclopropyl-N-[1-[8-[(8-methoxy-1,3-dimethyl-pyrrolo[1,2-a]pyrazin-7-yl)carbamoyl]-2-methyl-imidazo[1,2-a]pyridin-5-yl]-4-piperidyl]carbamate (B160, 38 mg, 64.7 μmol, 59% yield) as a solid. LCMS (ES, m/z): 588 [M+H]⁺.

### Synthesis of Compound 631

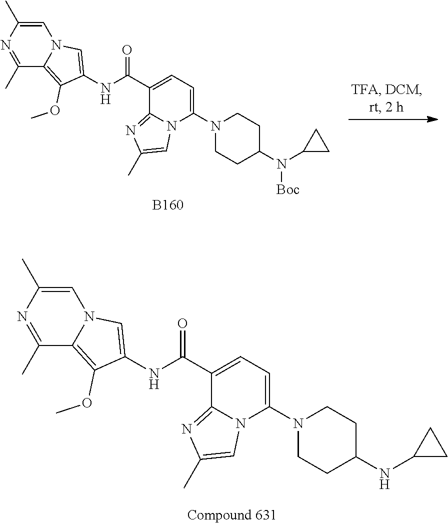 [SMILES: "B160" ; COc1c(NC(=O)c2ccc(N3CCC(N(C(=O)OC(C)(C)C)C4CC4)CC3)n3cc(C)nc23)cn2cc(C)nc(C)c12 ; COc1c(NC(=O)c2ccc(N3CCC(NC4CC4)CC3)n3cc(C)nc23)cn2cc(C)nc(C)c12]

To a stirred mixture of tert-butyl N-cyclopropyl-N-[1-[8-[(8-methoxy-1,3-dimethyl-pyrrolo[1,2-a]pyrazin-7-yl)carbamoyl]-2-methyl-imidazo[1,2-a]pyridin-5-yl]-4-piperidyl]-carbamate (33 mg, 56.15 μmol) in DCM (2 mL) was added TFA (0.5 mL) dropwise, and the reaction was stirred for 2 h at room temperature. The resulting mixture was concentrated under reduced pressure and purified by Prep-HPLC (Condition 5, Gradient 15) to afford 5-[4-(cyclopropylamino)-1-piperidyl]-N-(8-methoxy-1,3-dimethyl-pyrrolo[1,2-a]pyrazin-7-yl)-2-methyl-imidazo[1,2-a]pyridine-8-carboxamide (Compound 631, 11 mg, 22.6 μmol, 40% yield) as a solid. LCMS (ES, m/z): 488 [M+H]⁺. ¹H NMR (400 MHz, DMSO-d₆) δ 12.74 (s, 1H), 8.11 (s, 1H), 8.08 (d, J=7.9 Hz, 1H), 7.85 (s, 1H), 7.59 (d, J=1.1 Hz, 1H), 6.60 (d, J=7.9 Hz, 1H), 4.01 (s, 3H), 3.48 (d, J=12.0 Hz, 2H), 2.88 (t, J=11.3 Hz, 2H), 2.78 (s, 1H), 2.63 (s, 3H), 2.50 (s, 3H), 2.20 (d, J=1.0 Hz, 3H), 2.11 (tt, J=6.5, 3.5 Hz, 1H), 2.03 (d, J=12.7 Hz, 2H), 1.57 (q, J=11.3 Hz, 2H), 0.39 (dt, J=6.2, 3.0 Hz, 2H), 0.28-0.15 (m, 2H).

An analogous method was followed to obtain the following compounds.

| Compound | Starting Material | Characterization |
|---|---|---|
| 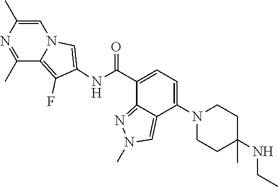 [SMILES: CCNC1(C)CCN(c2ccc(C(=O)Nc3cn4cc(C)nc(C)c4c3F)c3nn(C)cc23)CC1] Compound 571 | 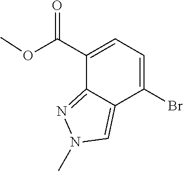 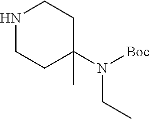 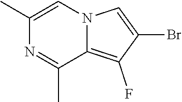 | Modification: Step 1 was ran at 90° C. for 16 h. Step 2 was ran at 50° C. for 3 h. Step 3 was ran using NH₄Cl, HATU and DIEA in DMF for 3 h at room temperature. Step 4 was ran for 5 h at 100° C. Step 5 was ran for 1 h. The crude product was purified by Prep-HPLC (Condition 5, Gradient 32) to afford 4-[4-(ethylamino)-4- methyl-1-piperidyl]-N-(8-fluoro-1,3- dimethyl-pyrrolo[1,2-a]-pyrazin-7- yl)-2-methyl-indazole-7-carboxamide (Compound 571, 15 mg, 31.4 μmol). LCMS (ES, m/z): 478 [M + H]⁺. ¹H NMR (400 MHz, DMSO-d₆) δ 11.26 (s, 1H), 8.79 (s, 1H), 8.15 (d, J = 3.6 Hz, 1H), 7.98 (d, J = 8.1 Hz, 1H), 7.90 (s, 1H), 6.48 (d, J = 8.3 Hz, 1H), 4.25 (s, 3H), 3.60-3.42 (m, 4H), 2.62 (s, 3H), 2.55 (d, J = 7.1 Hz, 2H), 2.28- 2.23 (m, 3H), 1.76-1.66 (m, 2H), 1.60 (ddd, J = 13.1, 8.5, 4.4 Hz, 2H), 1.27 (d, J = 22.4 Hz, 1H), 1.11 (s, 3H), 1.04 (t, J = 7.0 Hz, 3H). |
| 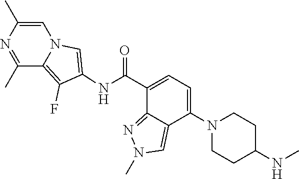 [SMILES: CNC1CCN(c2ccc(C(=O)Nc3cn4cc(C)nc(C)c4c3F)c3nn(C)cc23)CC1] Compound 581 | 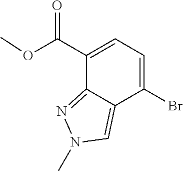 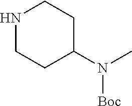 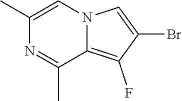 | Modification: Step 1 was ran at 90° C. Step 3 was ran using NH₄Cl, HATU and DIEA in DMF for 1 h at room temperature. Step 4 was ran for 16 h at 100° C. Step 5 was ran for 1 h. The residue was purified by reversed- phase flash chromatography (Condition 3, Gradient 26) to afford N-(8-fluoro-1,3-dimethyl- pyrrolo[1,2-a]-pyrazin-7-yl)-2- methyl-4-[4-(methylamino)-1- piperidyl]indazole-7-carboxamide (Compound 581, 10.2 mg, 22.3 μmol). LCMS (ES, m/z): 450 [M + H]⁺. ¹H NMR (400 MHz, DMSO-d₆) δ 11.26 (s, 1H), 8.79 (s, 1H), 8.16 (d, J = 3.6 Hz, 1H), 7.99 (d, J = 8.1 Hz, 1H), 7.90 (s, 1H), 6.50 (d, J = 8.2 Hz, 1H), 4.26 (s, 3H), 3.88 (d, J = 12.6 Hz, 2H), 3.07 (t, J = 11.8 Hz, 2H), 2.63 (s, 3H), 2.57 (dd, J = 9.2, 5.0 Hz, 1H), 2.33 (s, 3H), 2.24 (s, 3H), 2.00-1.92 (m, 2H), 1.44 (q, J = 11.1 Hz, 2H). |
| 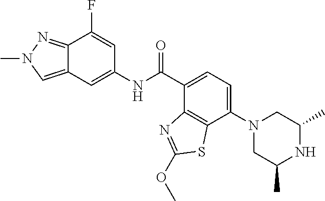 Compound 675 | 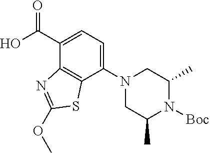 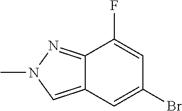 | Modification: The procedure begins on step 3 with the material to the left. Step 3 was ran using NH₄Cl, HATU and DIEA in DMF for 2 h at room temperature. Step 4 was ran for 3 h at 90° C. Step 5 was ran for 2 h. The residue was purified by reversed- phase flash chromatography (Condition 3, Gradient 26) to afford 7- [(3S,5S)-3,5-dimethylpiperazin-1-yl]- N-(7-fluoro-2-methyl-indazol-5-yl)- 2-methoxy-1,3-benzothiazole-4- carboxamide (Compound 675, 20 mg, 42.7 μmol). LCMS (ES, m/z): 481 [M + H]⁺. ¹H NMR (400 MHz, DMSO-d₆) δ 11.40 (s, 1H), 8.43 (d, J = 2.8 Hz, 1H), 8.15-8.06 (m, 2H), 7.34 (dd, J = 13.1, 1.7 Hz, 1H), 7.04 (d, J = 8.6 Hz, 1H), 4.40 (s, 3H), 4.19 (s, 3H), 3.22 (td, J = 6.4, 3.2 Hz, 2H), 3.09 (dd, |
| | | J = 11.4, 3.1 Hz, 2H), 2.91 (dd, J = |
| | | 11.4, 6.1 Hz, 2H), 2.17 (s, 1H), 1.16 |
| | | (d, J = 6.4 Hz, 6H). |

### Example 176: Synthesis of Compound 464

### Synthesis of B161

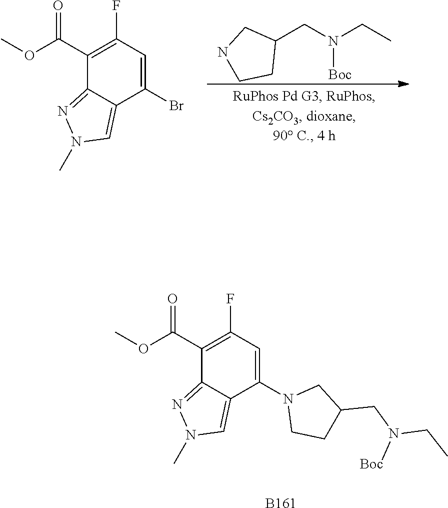

To a stirred solution of methyl 4-bromo-6-fluoro-2-methylindazole-7-carboxylate (150 mg, 0.697 mmol, 1 eq) and tert-butyl N-ethyl-N-(pyrrolidin-3-ylmethyl)carbamate (207 mg, 0.91 mmol, 1.3 eq) in dioxane (5 mL) were added Cs₂CO₃ (454 mg, 1.39 mmol, 2 eq), RuPhos (66 mg, 0.14 mmol, 0.2 eq) and RuPhos Pd G3 (59 mg, 0.07 mmol, 0.1 eq) at room temperature under N₂. The resulting mixture was stirred for 4 h at 90° C. under N₂. The resulting mixture was concentrated under reduced pressure and purified by silica gel column chromatography (67% EtOAc in PE) to afford 4-(3-([(tert-butoxycarbonyl)(ethyl)amino]methyl)pyrrolidin-1-yl)-6-fluoro-2-methylindazole-7-carboxylate (B161, 110 mg, 49% yield) as a solid. LCMS (ES, m/z): 435 [M+H]⁺.

### Synthesis of B162

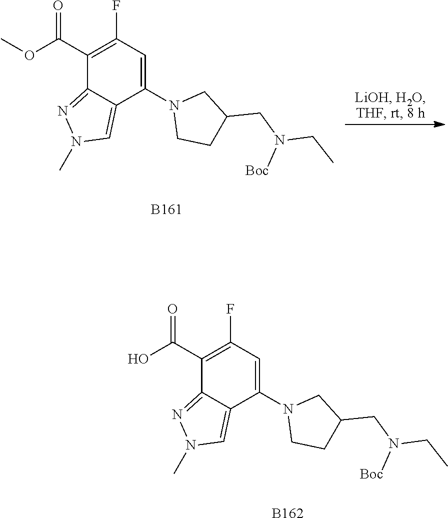 [SMILES: "B161" ; "B162" ; CCN(CC1CCN(c2cc(F)c(C(=O)OC)c3nn(C)cc23)C1)C(=O)OC(C)(C)C ; CCN(CC1CCN(c2cc(F)c(C(=O)O)c3nn(C)cc23)C1)C(=O)OC(C)(C)C ; "LiOH, H2O,THF, rt, 8 h"]

To a stirred solution of methyl 4-(3-([(tert-butoxycarbonyl)(ethyl)amino]-methyl)pyrrolidin-1-yl)-6-fluoro-2-methylindazole-7-carboxylate (110 mg, 0.25 mmol, 1 eq) in H₂O (1 mL) and THF (2 mL) was added LiOH·H₂O (64 mg, 1.52 mmol, 6 eq), and the reaction was stirred for 8 h at room temperature. The resulting mixture was acidified to pH 3 with citric acid and extracted with EtOAc (3×10 mL). The combined organic layers were washed with brine (5 mL), dried over anhydrous Na₂SO₄, and filtered. The filtrate was concentrated under reduced pressure to afford 4-(3-([(tert-butoxycarbonyl)(ethyl)amino]methyl)pyrrolidin-1-yl)-6-fluoro-2-methylindazole-7-carboxylic acid (B162, 70 mg, 66% yield) as a solid. LCMS (ES, m/z): 420 [M+H]⁺.

### Synthesis of B163

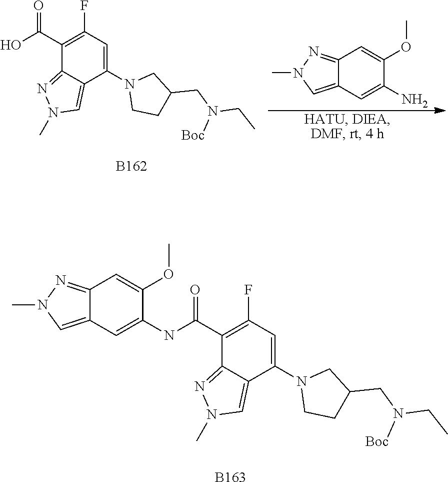

To a stirred solution of 4-(3-([(tert-butoxycarbonyl)(ethyl)amino]methyl)pyrrolidin-1-yl)-6-fluoro-2-methylindazole-7-carboxylic acid (70 mg, 0.17 mmol, 1 eq) and DIEA (108 mg, 0.83 mmol, 5 eq) in DMF (3 mL) were added HATU (76 mg, 0.199 mmol, 1.2 eq) and 6-methoxy-2-methylindazol-5-amine (33 mg, 0.18 mmol, 1.1 eq), and the reaction was stirred for 4 h at room temperature. The resulting mixture was quenched by the addition of water (12 mL) at room temperature. The precipitated solids were collected by filtration and washed with water (2×5 mL), to afford tert-butyl N-ethyl-N-[(1-(6-fluoro-7-[(6-methoxy-2-methylindazol-5-yl)carbamoyl]-2-methylindazol-4-yl)pyrrolidin-3-yl)methyl]carbamate (B163, 80 mg, 83% yield) as a solid. LCMS (ES, m/z): 580 [M+H]⁺.

### Synthesis of Compound 464

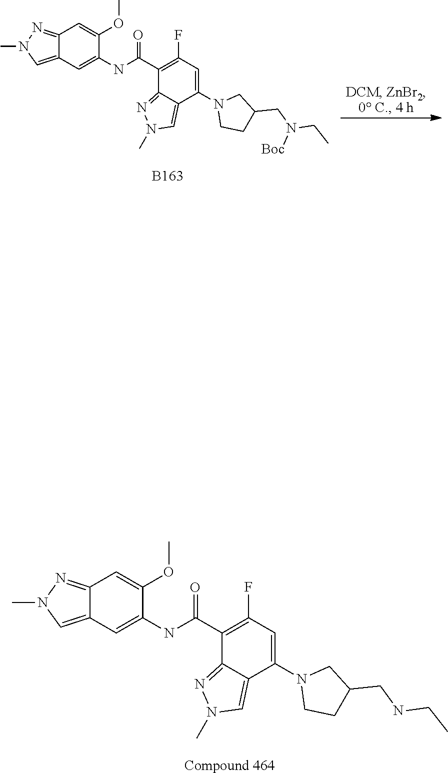

A solution of tert-butyl N-ethyl-N-[(1-(6-fluoro-7-[(6-methoxy-2-methylindazol-5-yl)carbamoyl]-2-methylindazol-4-yl)pyrrolidin-3-yl)methyl]carbamate (80 mg, 0.14 mmol, 1 eq) and ZnBr₂ (311 mg, 1.38 mmol, 10 eq) in DCM (2.5 mL) was stirred for 4 h at 0° C. The resulting mixture was concentrated under reduced pressure and purified by reversed-phase flash chromatography (Condition 3, Gradient 7) to afford 4-(3-[(ethylamino)methyl]pyrrolidin-1-yl)-6-fluoro-N-(6-methoxy-2-methylindazol-5-yl)-2-methylindazole-7-carboxamide (Compound 464, 12 mg, 18% yield) as a solid. LCMS (ES, m/z): 480 [M+H]⁺. ¹H NMR (300 MHz, DMSO-d₆) δ 11.59 (s, 1H), 8.79 (d, J=2.3 Hz, 2H), 8.15 (s, 1H), 7.04 (s, 1H), 5.74 (d, J=16.4 Hz, 1H), 4.22 (s, 3H), 4.05 (d, J=15.5 Hz, 6H), 3.75 (s, 1H), 3.67 (s, 2H), 2.63-2.53 (m, 4H), 2.16 (s, 1H), 1.80 (s, 1H), 1.24 (s, 1H), 1.04 (t, J=7.1 Hz, 3H).

An analogous method was followed to obtain the following compounds.

| | Starting | |
|---|---|---|
| Compound | Material | Characterization |
| 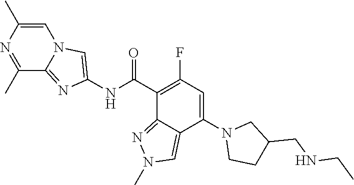 [SMILES: CCNCC1CCN(c2cc(F)c(C(=O)Nc3cn4cc(C)nc(C)c4n3)c3nn(C)cc23)C1] Compound 471 | 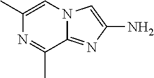 | The residue was purified by reversed- phase flash chromatography (Condition 3, Gradient 32) to afford N-(6,8- dimethylimidazo-[1,2-a]-pyrazin-2-yl)- 4-(3-[(ethylamino)methyl]-pyrrolidin-1- yl)-6-fluoro-2-methyl-indazole-7- carboxamide (Compound 471, 25 mg). LCMS (ES, m/z): 465 [M + H]⁺ ¹H NMR (300 MHz, DMSO-d₆) δ 11.54 (s, 1H), 8.82 (s, 1H), 8.28 (d, J = 10.0 Hz, 2H), 5.78 (d, J = 16.3 Hz, 1H), 4.23 (s, 3H), 3.69 (dd, J = 28.2, 13.8 Hz, 4h), 2.69 (s, 3H), 2.58 (dd, J = 10.3, 7.2 Hz, 4H), 2.37 (d, J = 1.0 Hz, 4H), 2.15 (s, 1H), 1.79 (dd, |
| | | J = 12.2, 7.8 Hz, 1H), 1.04 (t, J = 7.1 Hz, |
| | | 3H). |

### Example 177: Synthesis of C180

### Synthesis of C179

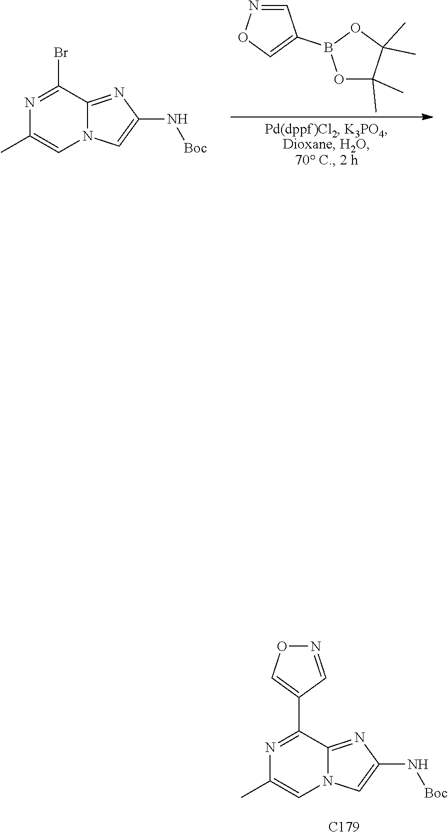 [SMILES: Cc1cn2cc(NC(=O)OC(C)(C)C)nc2c(Br)n1 ; CC1(C)OB(c2cnoc2)OC1(C)C ; "Pd(dppf)Cl2, K3PO4,Dioxane, H2O,70° C., 2 h" ; Cc1cn2cc(NC(=O)OC(C)(C)C)nc2c(-c2cnoc2)n1 ; "C179"]

To a solution of tert-butyl N-(8-bromo-6-methyl-imidazo[1,2-a]pyrazin-2-yl)carbamate (120.0 mg, 366.8 μmol) and 4-(4,4,5,5-tetramethyl-1,3,2-dioxaborolan-2-yl) isoxazole (85.8 mg, 440.1 μmol) in Dioxane (2 mL) and H₂O (0.3 mL) were added K₃PO₄ (155.7 mg, 733.6 μmol) and Pd(dppf)Cl₂ (26.8 mg, 36.68 μmol), and the reaction was stirred for 2 h at 70° C. under N₂. The resulting mixture was concentrated under reduced pressure and purified by silica gel column chromatography (67% EtOAc in PE) to afford tert-butyl (8-(isoxazol-4-yl)-6-methylimidazo[1,2-a]pyrazin-2-yl)carbamate (C179, 60 mg, 189.9 μmol, 52% yield) as a solid. LCMS (ES, m/z): 316 [M+H]⁺.

### Synthesis of C179

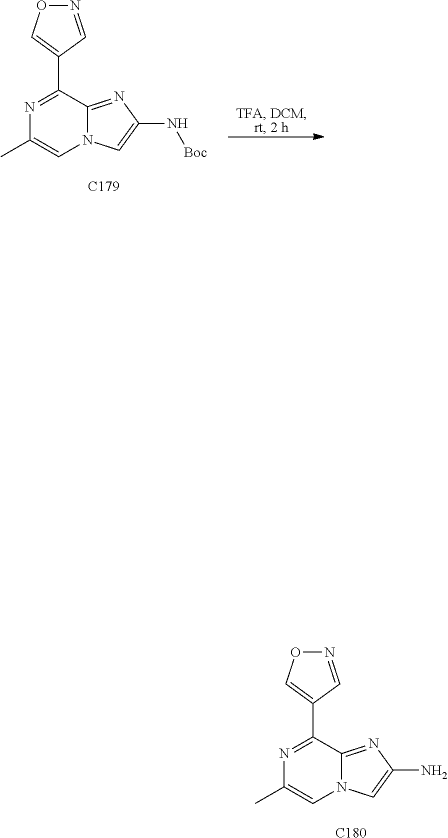 [SMILES: Cc1cn2cc(NC(=O)OC(C)(C)C)nc2c(-c2cnoc2)n1 ; "TFA, DCM,rt, 2 h" ; "C179" ; Cc1cn2cc(N)nc2c(-c2cnoc2)n1 ; "C180"]

To a solution of tert-butyl N-(8-isoxazol-4-yl-6-methyl-imidazo[1,2-a]pyrazin-2-yl)carbamate (55.0 mg, 174.4 μmol) in DCM (1 mL) was added TFA (0.4 mL), and the reaction was stirred for 2 h at rt. The resulting mixture was concentrated under reduced pressure and adjusted to pH 8 with saturated NaHCO₃ (aq.). The resulting mixture was diluted with water (5 mL) and extracted with DCM/MeOH (20/1) (2×10 mL). The combined organic layers were washed with water (20 mL), brine (20 mL) and dried over anhydrous Na₂SO₄, and filtered. The filtrate was concentrated under reduced pressure to afford 8-isoxazol-4-yl-6-methyl-imidazo[1,2-a]pyrazin-2-amine (C179, 36 mg, 167.3 μmol, 96% yield) as a solid. LCMS (ES, m/z): 216 [M+H]⁺.

### Example 178: Synthesis of Compound 458

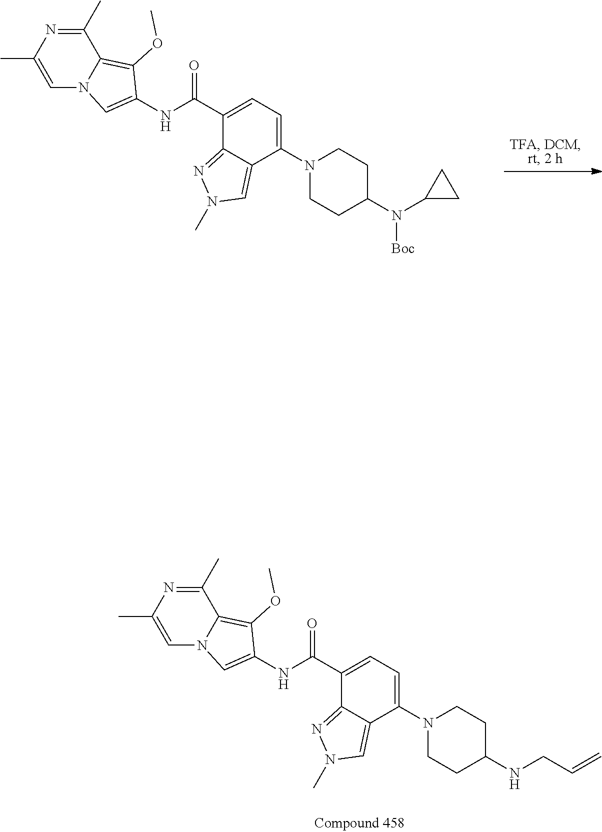 [SMILES: COc1c(NC(=O)c2ccc(N3CCC(N(C(=O)OC(C)(C)C)C4CC4)CC3)c3cn(C)nc23)cn2cc(C)nc(C)c12 ; "TFA, DCM,rt, 2 h" ; C=CCNC1CCN(c2ccc(C(=O)Nc3cn4cc(C)nc(C)c4c3OC)c3nn(C)cc23)CC1]

To a stirred mixture of tert-butyl N-cyclopropyl-N-[1-[7-[(8-methoxy-1,3-dimethyl-pyrrolo[1,2-a]pyrazin-7-yl)carbamoyl]-2-methyl-indazol-4-yl]-4-piperidyl]carbamate (28 mg, 47.64 μmol) in DCM (1 mL) was added TFA (0.3 mL) dropwise, and the reaction was stirred for 2 h at room temperature. The resulting mixture was concentrated under reduced pressure and purified by CHIRAL-HPLC (Condition 4, Gradient 1) to afford 4-[4-(allylamino)-1-piperidyl]-N-(8-methoxy-1,3-dimethyl-pyrrolo[1,2-a]pyrazin-7-yl)-2-methyl-indazole-7-carboxamide (Compound 458, 4.7 mg, 9.6 μmol, 20% yield) as a solid. LCMS (ES, m/z): 488 [M+H]⁺. ¹H NMR (300 MHz, Methanol-d₄) δ 8.50 (s, 1H), 8.11 (s, 1H), 8.08 (d, J=8.1 Hz, 1H), 7.70 (s, 1H), 6.54 (d, J=8.2 Hz, 1H), 5.97 (dd, J=17.1, 10.3 Hz, 1H), 5.24 (dd, J=28.2, 13.7 Hz, 2H), 4.35 (s, 3H), 4.11 (s, 3H), 4.02 (d, J=12.9 Hz, 2H), 3.36 (d, J=6.9 Hz, 2H), 3.03 (t, J=12.4 Hz, 2H), 2.85 (d, J=11.2 Hz, 1H), 2.75 (s, 3H), 2.31 (s, 3H), 2.20-2.06 (m, 2H), 1.63 (q, J=11.6, 11.0 Hz, 2H).

### Example 179: Synthesis of Compound 627

### Synthesis of B164

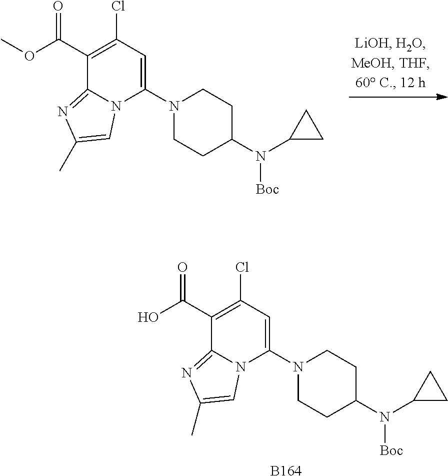 [SMILES: COC(=O)c1c(Cl)cc(N2CCC(N(C(=O)OC(C)(C)C)C3CC3)CC2)n2cc(C)nc12 ; "B164" ; Cc1cn2c(N3CCC(N(C(=O)OC(C)(C)C)C4CC4)CC3)cc(Cl)c(C(=O)O)c2n1]

To a stirred mixture of methyl 5-[4-[tert-butoxycarbonyl(cyclopropyl)amino]-1-piperidyl]-7-chloro-2-methyl-imidazo[1,2-a]pyridine-8-carboxylate (0.22 g, 475.2 μmol) in MeOH (1 mL), THF (1 mL) and H₂O (0.5 mL) was added LiOH (34 mg, 1.43 mmol), and the reaction was stirred for 12 h at 60° C. The mixture residue was neutralized to pH 7 with 1 N of HCl (aq.)., diluted with water (3 mL), and extracted with EtOAc (2×8 mL). The combined organic layers were washed with water (2×10 mL), dried over anhydrous Na₂SO₄, and filtered. The filtrate was concentrated under reduced pressure, to afford 5-[4-[tert-butoxycarbonyl-(cyclopropyl)amino]-1-piperidyl]-7-chloro-2-methyl-imidazo[1,2-a]-pyridine-8-carboxylic acid (B164, 0.075 g, 167.1 μmol, 35% yield) as a solid. LCMS (ES, m/z): 449 [M+H]⁺.

### Synthesis of B165

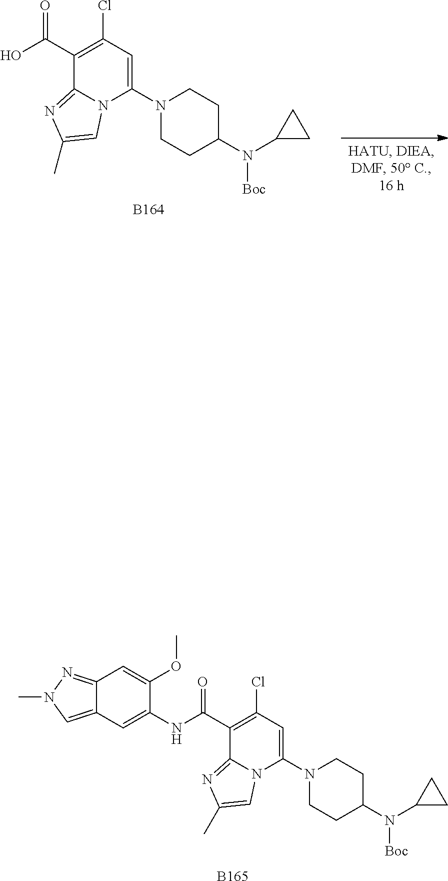 [SMILES: "B164" ; Cc1cn2c(N3CCC(N(C(=O)OC(C)(C)C)C4CC4)CC3)cc(Cl)c(C(=O)O)c2n1 ; "B165" ; COc1cc2nn(C)cc2cc1NC(=O)c1c(Cl)cc(N2CCC(N(C(=O)OC(C)(C)C)C3CC3)CC2)n2cc(C)nc12]

To a stirred mixture of 5-[4-[tert-butoxycarbonyl(cyclopropyl)amino]-1-piperidyl]-7-chloro-2-methyl-imidazo[1,2-a]pyridine-8-carboxylic acid (0.075 g, 167.1 μmol) and 6-methoxy-2-methyl-indazol-5-amine (35 mg, 200.5 μmol) in DMF (1 mL) were added DIEA (64 mg, 501.2 μmol), HATU (95 mg, 250.6 μmol), and the reaction was stirred for 16 h at 50° C. The mixture was allowed to cool down to room temperature. The resulting mixture was diluted with water (3 mL) and extracted with EtOAc (2×4 mL). The combined organic layers were washed with water (2×6 mL), dried over anhydrous Na₂SO₄, and filtered. The filtrate was concentrated under reduced pressure and purified by silica gel column chromatography (91% EtOAc in PE) to afford tert-butyl N-[1-[7-chloro-8-[(6-methoxy-2-methyl-indazol-5-yl)carbamoyl]-2-methyl-imidazo[1,2-a]pyridin-5-yl]-4-piperidyl]-N-cyclopropyl-carbamate (B165, 0.01 g, 16.4 μmol, 10% yield) as a solid. LCMS (ES, m/z): 608 [M+H]⁺.

### Synthesis of Compound 627

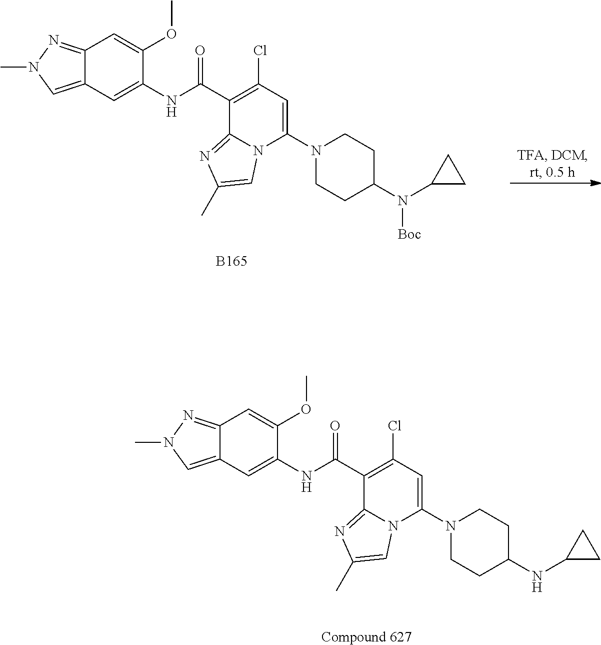 [SMILES: "B165" ; COc1cc2nn(C)cc2cc1NC(=O)c1c(Cl)cc(N2CCC(N(C(=O)OC(C)(C)C)C3CC3)CC2)n2cc(C)nc12 ; COc1cc2nn(C)cc2cc1NC(=O)c1c(Cl)cc(N2CCC(NC3CC3)CC2)n2cc(C)nc12 ; "Compound 627"]

To a solution of tert-butyl N-[1-[7-chloro-8-[(6-methoxy-2-methyl-indazol-5-yl)carbamoyl]-2-methyl-imidazo[1,2-a]pyridin-5-yl]-4-piperidyl]-N-cyclopropyl-carbamate (0.01 g, 16.44 μmol) in DCM (0.5 mL) was added TFA (0.2 mL), and the reaction was stirred for 0.5 h at rt. The resulting mixture was filtered and concentrated under reduced pressure to afford 7-chloro-5-(4-(cyclopropylamino)piperidin-1-yl)-N-(6-methoxy-2-methyl-2H-indazol-5-yl)-2-methylimidazo[1,2-a]pyridine-8-carboxamide 2,2,2-trifluoroacetate (Compound 627, 0.003 g, 3.22 μmol, 20% yield) as a solid. LCMS (ES, m/z): 508 [M+H]⁺. ¹H NMR (400 MHz, Methanol-d₄) δ 8.66 (s, 1H), 8.15 (s, 1H), 7.73 (d, J=7.2 Hz, 1H), 7.22 (s, 1H), 7.08 (s, 1H), 4.16 (s, 3H), 3.95 (s, 3H), 3.54 (d, J=12.0 Hz, 2H), 3.40 (d, J=12.3 Hz, 1H), 2.91 (t, J=12.0 Hz, 2H), 2.62 (s, 3H), 2.02 (d, J=12.6 Hz, 3H), 1.68 (q, J=12.2 Hz, 2H), 1.29 (s, 2H), 0.88 (s, 1H), 0.55 (s, 2H), 0.46 (d, J=7.1 Hz, 2H).

### Example 180: Synthesis of C184

### Synthesis of C181

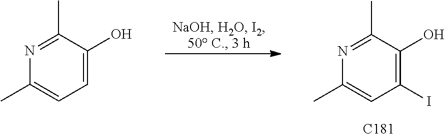

To a stirred mixture of 2,6-dimethylpyridin-3-ol (5 g, 40.6 mmol) in H₂O (150 mL) were added NaOH (8.12 g, 203.0 mmol) and I₂ (13.4 g, 52.78 mmol), and the reaction was stirred for 3 h at 50° C. The resulting mixture was diluted with water (200 mL), acidified to pH 6 with HCl (2 N), and extracted with EtOAc (2×200 mL). The combined organic layers were washed with brine (200 mL), dried over anhydrous Na₂SO₄, and filtered. The filtrate was concentrated under reduced pressure and purified by silica gel column chromatography (50% EtOAc in PE) to afford 4-iodo-2,6-dimethyl-pyridin-3-ol (C181, 2.2 g, 8.83 mmol, 22% yield) as an oil. LCMS (ES, m/z): 250 [M+H]⁺.

### Synthesis of C182

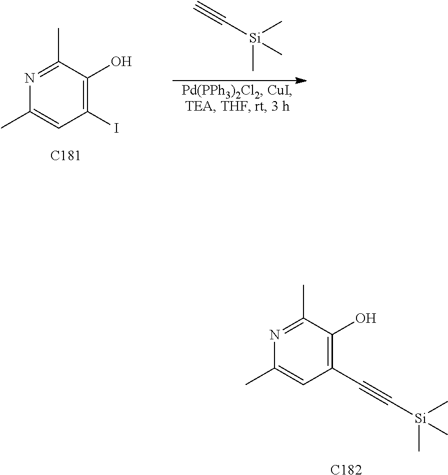

To a solution of 4-iodo-2,6-dimethyl-pyridin-3-ol (2.2 g, 8.83 mmol) and ethynyl(trimethyl)silane (1.74 g, 17.67 mmol, 2.50 mL) in THF (20 mL) were added TEA (893.8 mg, 8.83 mmol), CuI (168.2 mg, 883.4 μmol) and Pd(PPh₃)₂Cl₂ (620.1 mg, 883.4 μmol), and the reaction was stirred for 3 h at rt under N₂. The resulting mixture was concentrated under reduced pressure and purified by silica gel column chromatography (50% EtOAc in PE) to afford 2,6-dimethyl-4-(2-trimethylsilylethynyl)pyridin-3-ol (C182, 1.5 g, 6.8 mmol, 77% yield) as a solid. LCMS (ES, m/z): 220 [M+H]⁺.

### Synthesis of C183

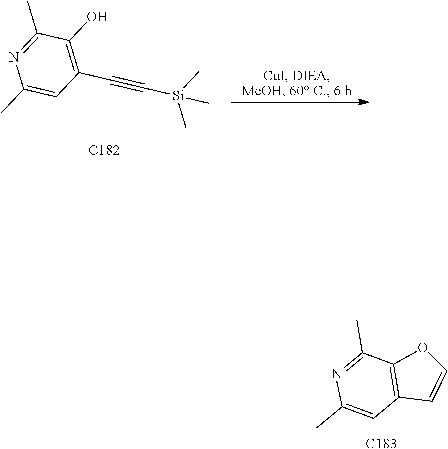

To a stirred mixture of 2-methyl-4-(2-trimethylsilylethynyl)pyridin-3-ol (1.4 g, 6.82 mmol) in MeOH (25 mL) was added DIEA (2.64 g, 20.46 mmol) and CuI (129.8 mg, 681.8 μmol), and the reaction was stirred for 6 h at 60° C. under N₂. The reaction was quenched with Water (100 ml) and extracted with EtOAc (2×100 mL). The combined organic layers were washed with brine (100 mL), dried over anhydrous Na₂SO₄, and filtered. The filtrate was concentrated under reduced pressure and purified by silica gel column chromatography (50% EtOAc in PE) to afford 5,7-dimethylfuro[2,3-c]pyridine (C183, 800 mg, 5.4 mmol, 80% yield) as a solid. LCMS (ES, m/z): 148 [M+H]⁺.

### Synthesis of C184

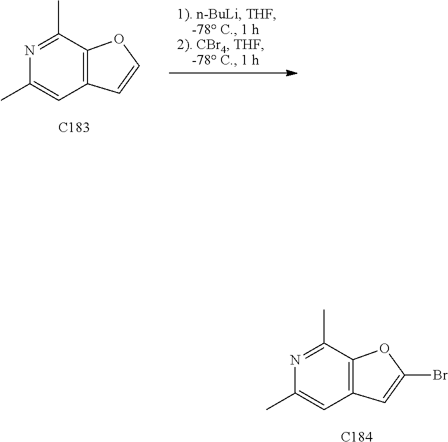

In a 50-mL round bottom flask, to a solution of 5,7-dimethylfuro[2,3-c]pyridine (800 mg, 5.44 mmol) in THF (10 mL) was added n-BuLi (2.5 M in hexane) (4.3 mL, 10.87 mmol) dropwise at −78° C. under N₂ atmosphere. The reaction mixture was stirred at −78° C. for 1 h. Followed by the addition of CBr₄ (2.67 g, 8.15 mmol) in THF (5 mL), the mixture was stirred for additional 1 h. The reaction was quenched with saturated NH₄Cl aqueous (100 mL), and then the mixture was extracted with EtOAc (100 mL). The combined organic extracts were washed with brine (100 mL), dried over anhydrous Na₂SO₄, and filtered. The filtrate was concentrated under reduced pressure and purified by silica gel column chromatography (50% EtOAc in PE) to afford 2-bromo-5,7-dimethyl-furo[2,3-c]pyridine (C184, 400 mg, 1.77 mmol, 33% yield) as a solid. LCMS (ES, m/z): 148 [M+H]⁺.

An analogous method was followed to obtain the following intermediate.

| | Starting | |
|---|---|---|
| Compound | Material | Characterization |
| 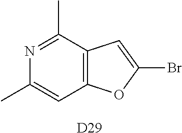 | 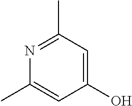 | Modifications: Step 1 was ran at room temperature for 1 h. Step 2 was ran in DMF for 2 h at 70° C. Step 3 was ran for 1 h. Step 4-1 was ran for 0.5 h. Step 4-2 was ran at −90° C. The residue was purified by silica gel column chromatography (25% EtOAc in PE) to afford 2-bromo- 4,6-dimethyl-furo[3,2-c]pyridine (D49, 120 mg, 530.8 μmol). LCMS (ES, m/z): 226 [M + H]⁺. |

### Example 181: Synthesis of C188

### Synthesis of C185

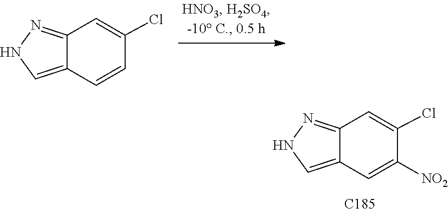 [SMILES: Clc1ccc2c[nH]nc2c1 ; O=[N+]([O-])c1cc2c[nH]nc2cc1Cl ; "C185"]

To a mixture of 6-chloro-2H-indazole (10 g, 65.54 mmol) in H₂SO₄ (40 mL) was added conc. HNO₃ (8 mL) slowly at −10° C. The mixture was stirred for 30 min at −10° C. The reaction was quenched with cold water. The precipitated solids were collected by filtration and washed with water. The crude product was purified by silica gel column chromatography (33% EtOAc in PE) to afford 6-chloro-5-nitro-2H-indazole (C185, 6 g, 30.4 mmol, 46% yield) as a solid. LCMS (ES, m/z): 196 [M−H]⁻.

### Synthesis of C186

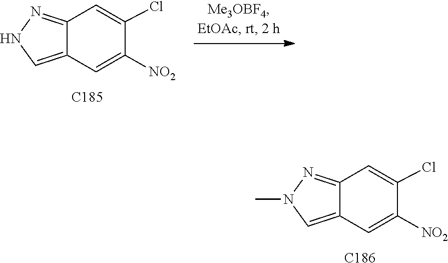

To a solution of 6-chloro-5-nitro-2H-indazole (5 g, 25.3 mmol) in EtOAc (50 mL) was added Me₃OBF₄ (4.12 g, 27.84 mmol), and the reaction was stirred for 2 h at rt. The reaction was diluted with H₂O (100 mL) and extracted with EtOAc (2×100 mL). The combined organic layers were washed with H₂O (100 mL), dried over anhydrous Na₂SO₄, and filtered. The filtrate was concentrated under reduced pressure and purified by silica gel column chromatography (25% EtOAc in PE) to afford 6-chloro-2-methyl-5-nitro-indazole (C186, 2.5 g, 11.8 mmol, 47% yield) as a solid. LCMS (ES, m/z): 212 [M+H]⁺.

### Synthesis of C187

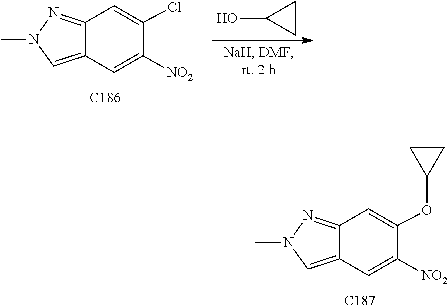

To a solution of cyclopropanol (230.5 mg, 3.97 mmol) in DMF (10 mL) was added NaH (60% dispersion in oil) (198.4 mg, 4.96 mmol) at 0° C. under N₂. The mixture was stirred for 0.5 h at 0° C. Followed by the addition of 6-chloro-2-methyl-5-nitro-indazole (700 mg, 3.31 mmol), the mixture was stirred for 2 h at rt. The reaction was quenched with H₂O (25 mL) and extracted with EtOAc (2×25 mL). The combined organic layers were washed with H₂O (2×25 mL), dried over anhydrous Na₂SO₄, and filtered. The filtrate was concentrated under reduced pressure and purified by silica gel column chromatography (20% EtOAc in PE) to afford 6-(cyclopropoxy)-2-methyl-5-nitro-indazole (C187, 85 mg, 364.5 μmol, 11% yield) as an oil. LCMS (ES, m/z): 234 [M+H]⁺.

### Synthesis of C188

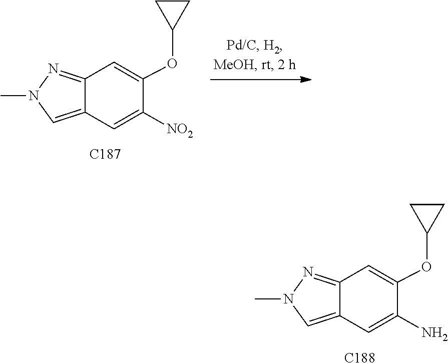 [SMILES: Cn1cc2cc([N+](=O)[O-])c(OC3CC3)cc2n1 ; "C187" ; Cn1cc2cc(N)c(OC3CC3)cc2n1 ; "C188"]

To a solution of 6-(cyclopropoxy)-2-methyl-5-nitro-indazole (85 mg, 364.5 μmol) in MeOH (10 mL) was added Pd/C (10% on Carbon (wetted with ca. 55% water)) (15.5 mg) under N₂. The mixture was stirred for 3 h at rt under H₂. The mixture was filtered through a Celite pad and concentrated under reduced pressure to afford 6-(cyclopropoxy)-2-methyl-indazol-5-amine (C188, 65 mg, 319.8 μmol, 88% yield) as an oil. LCMS (ES, m/z): 204 [M+H]⁺.

### Example 182: Synthesis of C190

### Synthesis of C189

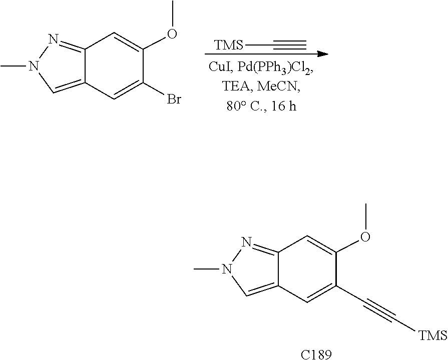

To a stirred mixture of 5-bromo-6-methoxy-2-methyl-indazole (0.8 g, 3.32 mmol) and ethynyl(trimethyl)silane (651 mg, 6.64 mmol) in MeCN (16 mL) were added CuI (63 mg, 331.8 μmol), Pd(dppf)Cl₂ (242 mg, 331.8 μmol) and TEA (1.0 g, 9.96 mmol), and the reaction was stirred for 16 h at 80° C. under N₂. The resulting mixture was concentrated under reduced pressure and purified by silica gel column chromatography (50% EtOAc in PE) to afford 2-(6-methoxy-2-methyl-indazol-5-yl)ethynyl-trimethyl-silane (C189, 660 mg, 2.55 mmol, 77% yield) as a solid. LCMS (ES, m/z): 259 [M+H]⁺.

### Synthesis of C190

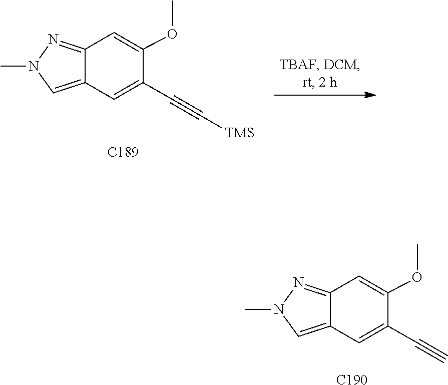 [SMILES: COc1cc2nn(C)cc2cc1C#C[Si](C)(C)C ; "C189" ; C#Cc1cc2cn(C)nc2cc1OC ; "C190"]

A solution of 2-(6-methoxy-2-methyl-indazol-5-yl)ethynyl-trimethyl-silane (600 mg, 2.32 mmol) in DCM (6 mL) was treated with TBAF (648 mg, 2.32 mmol) at rt. The resulting mixture was stirred for 2 h at room temperature. The resulting mixture was diluted with water (20 mL) and extracted with DCM (3×30 mL). The combined organic layers were washed with water (2×40 mL) and brine (40 mL), dried over anhydrous Na₂SO₄, and filtered. The filtrate was concentrated under reduced pressure, to afford 5-ethynyl-6-methoxy-2-methyl-indazole (C190, 270 mg, 1.45 mmol, 62% yield) as a solid. LCMS (ES, m/z): 187 [M+H]⁺.

### Example 183: Synthesis of Compound 437

### Synthesis of B167

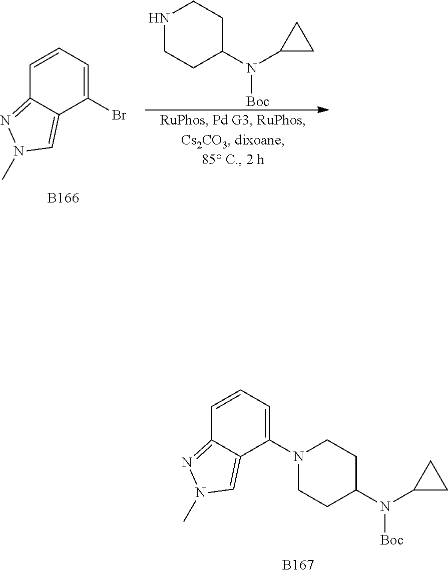

To a stirred mixture of 4-bromo-2-methyl-indazole (1 g, 4.74 mmol) and tert-butyl N-cyclopropyl-N-(4-piperidyl)carbamate (1.37 g, 5.69 mmol) in dioxane (20 mL) were added Cs₂CO₃ (4.63 g, 14.21 mmol), RuPhos (442 mg, 947.6 μmol) and RuPhos Pd G3 (396 mg, 473.8 μmol), and the reaction was stirred for 2 h at 85° C. under N₂. The resulting mixture was allowed to cool to room temperature and purified by silica gel column chromatography (80% EtOAc in PE) to afford tert-butyl N-cyclopropyl-N-[1-(2-methylindazol-4-yl)-4-piperidyl]carbamate (B167, 1.5 g, 3.04 mmol, 64% yield, 75% purity) as a solid. LCMS (ES, m/z): 371 [M+H]⁺.

### Synthesis of B168

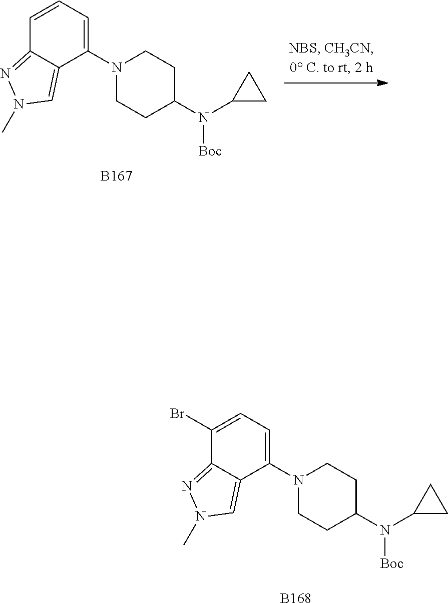 [SMILES: "B167" ; "B168" ; Cn1cc2c(N3CCC(N(C(=O)OC(C)(C)C)C4CC4)CC3)cccc2n1 ; Cn1cc2c(N3CCC(N(C(=O)OC(C)(C)C)C4CC4)CC3)ccc(Br)c2n1]

A solution of tert-butyl N-cyclopropyl-N-[1-(2-methylindazol-4-yl)-4-piperidyl]carbamate (1.4 g, 3.78 mmol) in MeCN (24 mL) was treated with NBS (672 mg, 3.78 mmol) at 0° C. under N₂. The resulting mixture was stirred for 2 h at room temperature under N₂. The resulting mixture was diluted with water (20 mL) and extracted with EtOAc (3×20 mL). The combined organic layers were washed with brine (40 mL), dried over anhydrous Na₂SO₄, and filtered. The filtrate was concentrated under reduced pressure, to afford tert-butyl N-[1-(7-bromo-2-methyl-indazol-4-yl)-4-piperidyl]-N-cyclopropyl-carbamate (B168, 1 g, 2.23 mmol, 59% yield) as a solid. LCMS (ES, m/z): 449 [M+H]⁺.

### Synthesis of B169

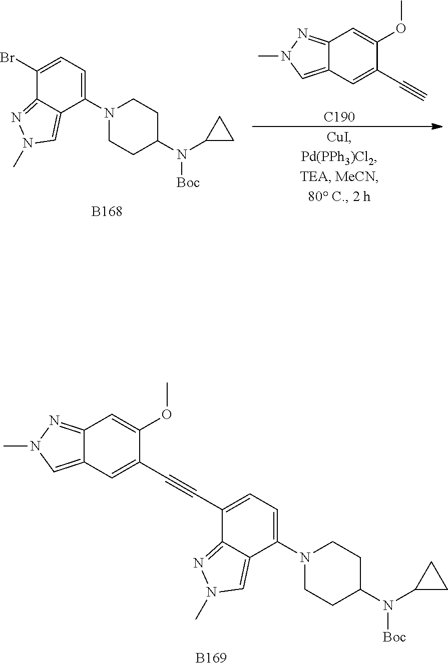 [SMILES: "C190" ; "B168" ; "B169" ; COc1cc2nn(C)cc2cc1C#Cc1ccc(N2CCC(N(C(=O)OC(C)(C)C)C3CC3)CC2)c2cn(C)nc12 ; Cn1cc2c(N3CCC(N(C(=O)OC(C)(C)C)C4CC4)CC3)ccc(Br)c2n1 ; C#Cc1cc2cn(C)nc2cc1OC]

To a stirred mixture of tert-butyl N-[1-(7-bromo-2-methyl-indazol-4-yl)-4-piperidyl]-N-cyclopropyl-carbamate (120 mg, 267.0 μmol) and 5-ethynyl-6-methoxy-2-methyl-indazole (59 mg, 320.4 μmol) in MeCN (2 mL) were added CuI (5 mg, 26.7 μmol), Pd(PPh₃)₂Cl₂ (19.5 mg, 26.7 μmol) and TEA (243 mg, 2.40 mmol), and the reaction was stirred for 2 h at 80° C. under N₂. The resulting mixture was allowed to cool down to room temperature, concentrated under reduced pressure, and purified by silica gel column chromatography (83% EtOAc in PE) to afford tert-butyl N-cyclopropyl-N-[1-[7-[2-(6-methoxy-2-methyl-indazol-5-yl)ethynyl]-2-methyl-indazol-4-yl]-4-piperidyl]carbamate (B169, 58 mg, 104.6 μmol, 39% yield) as a solid. LCMS (ES, m/z): 555 [M+H]⁺.

### Synthesis of Compound 437

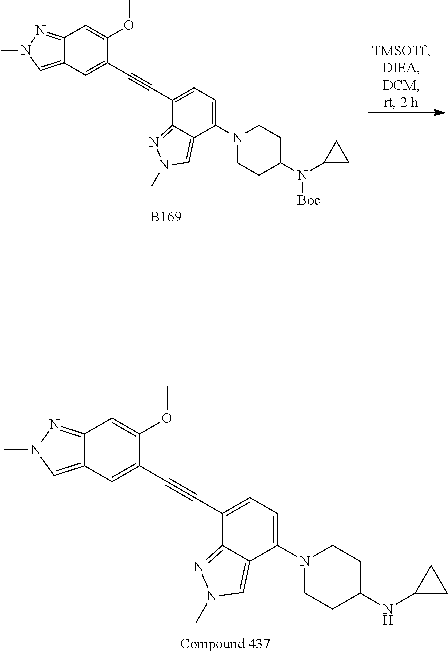

To a stirred mixture of tert-butyl N-cyclopropyl-N-[1-[7-[2-(6-methoxy-2-methyl-indazol-5-yl)ethynyl]-2-methyl-indazol-4-yl]-4-piperidyl]carbamate (46 mg, 82.93 μmol) and DIEA (53 mg, 414.7 μmol) in DCM (2 mL) was added TMSOTf (55 mg, 248.8 μmol) dropwise, and the reaction was stirred for 2 h at room temperature. The resulting mixture was concentrated under reduced pressure and purified by Prep-HPLC (Condition 5, Gradient 19a) to afford N-cyclopropyl-1-[7-[2-(6-methoxy-2-methyl-indazol-5-yl)ethynyl]-2-methyl-indazol-4-yl]-piperidin-4-amine (Compound 437, 10 mg, 22 μmol, 27% yield) as a solid. LCMS (ES, m/z): 455 [M+H]⁺. ¹H NMR (400 MHz, DMSO-d₆) δ 8.54 (s, 1H), 8.26 (s, 1H), 7.81 (s, 1H), 7.16 (d, J=8.8 Hz, 1H), 7.10 (d, J=8.8 Hz, 1H), 6.98 (s, 1H), 4.13 (s, 3H), 4.11 (s, 3H), 3.88 (s, 3H), 3.85 (s, 2H), 3.27 (t, J=12.2 Hz, 2H), 2.70 (d, J=19.4 Hz, 1H), 2.13 (dd, J=6.8, 3.5 Hz, 1H), 2.01 (d, J=12.3 Hz, 2H), 1.54 (q, J=10.5, 10.0 Hz, 2H), 0.39 (dt, J=6.1, 3.1 Hz, 2H), 0.23 (p, J=3.9 Hz, 2H).

An analogous method was followed to obtain the following compounds.

| | Starting | |
|---|---|---|
| Compound | Material | Characterization |
| 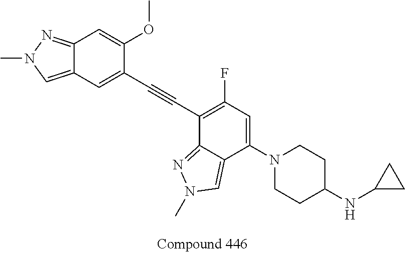 [SMILES: COc1cc2nn(C)cc2cc1C#Cc1c(F)cc(N2CCC(NC3CC3)CC2)c2cn(C)nc12] | 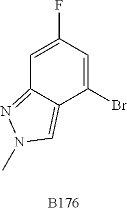 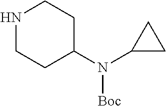 [SMILES: CC(C)(C)OC(=O)N(C1CCNCC1)C1CC1] 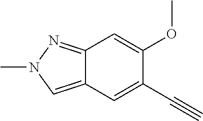 C190 | Modification: Step 1 was ran for 2 h at 85° C. Step 2 was ran using NIS and HOAc in DMF for 1 h at room temperature. Step 3 was ran for 50° C. for 16 h. Step 4 was ran for 2 h at room temperature. The residue was purified by reversed-phase flash chromatography (Condition 3, Gradient 11) to afford N-cyclo- propyl-1-[6-fluoro-7- [2-(6-methoxy-2-methyl- indazol-5-yl)-ethynyl]-2-methyl- indazol-4-yl]-piperidin-4-amine (Compound 446, 12.9 mg, 27.3 μmol, 20% yield) as a solid. LCMS (ES, m/z): 473 [M + H]⁻. ¹H NMR (400 MHz, DMSO-d₆) δ 8.58 (s, 1H), 8.27 (s, 1H), 7.86 (s, 1H), 7.00 (s, 1H), 6.24 (d, J = 13.4 Hz, 1H), 4.12 (d, J = 4.9 Hz, 6H), 3.87 (s, 3H), 3.82-3.74 (m, 2H), 2.97 (t, J = 11.7 Hz, 2H), 2.75 (s, 1H), 2.26 (s, 1H), 2.12 (s, 1H), 1.99 (d, J = 12.8 Hz, 2H), 1.49 (d, J = 10.6 Hz, 1H), 1.44 (d, J = 10.5 Hz, 1H), 0.40 (dt, J = 6.5, 3.3 Hz, 2H), 0.27-0.20 (m, 2H). |

### Example 184: Synthesis of C197

### Synthesis of C191

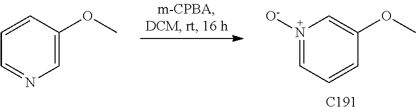

To a stirred mixture of 3-methoxypyridine (30 g, 274.9 mmol) in DCM (300 mL) was added m-CPBA (71.16 g, 412.4 mmol) at 0° C. The resulting mixture was stirred for 16 h at room temperature. The reaction was quenched with sat. Na₂SO₃ (aq.) at 0° C. The resulting mixture was extracted with DCM (3×200 mL). The combined organic layers were washed with brine (200 mL), dried over anhydrous Na₂SO₄, and filtered. The filtrate was concentrated under reduced pressure and purified by silica gel column chromatography (9% MeOH in DCM) to afford 3-methoxy-1-oxido-pyridin-1-ium (C191, 31 g, 247.8 mmol, 90% yield) as an oil. LCMS (ES, m/z): 126 [M+H]⁺.

### Synthesis of C192

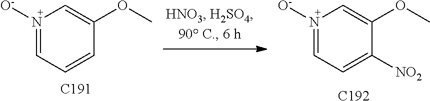

A solution of 3-methoxy-1-oxido-pyridin-1-ium (30 g, 239.8 mmol) in H₂SO₄ (conc.) (60 mL) was treated with HNO₃ (60 mL) for 10 min at 0° C. under N₂. The resulting mixture was stirred for 6 h at 90° C. The mixture was basified to pH 9 with Na₂CO₃. The resulting mixture was extracted with EtOAc (3×200 mL). The combined organic layers were washed with brine (300 mL), dried over anhydrous Na₂SO₄, and filtered. The filtrate was concentrated under reduced pressure and purified by silica gel column chromatography (67% EtOAc in PE) to afford 3-methoxy-4-nitro-1-oxido-pyridin-1-ium (C192, 22 g, 129.3 mmol, 54% yield) as a solid. LCMS (ES, m/z): 171 [M+H]⁺.

### Synthesis of C193

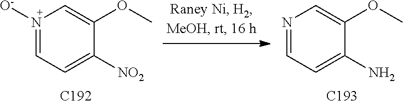

To a solution of 3-methoxy-4-nitro-1-oxido-pyridin-1-ium (22 g, 129.3 mmol) in MeOH (500 mL) was added Raney Ni (20%, 4 g) under N₂ in a 500 mL round-bottom flask. The mixture was hydrogenated at room temperature for 16 h under hydrogen atmosphere using a hydrogen balloon. The insoluble solids were filtered off, and the filtrate was concentrated under reduced pressure to afford 3-methoxypyridin-4-amine (C193, 16 g, 128.9 mmol, 99.7% yield) as a solid. LCMS (ES, m/z): 125 [M+H]⁺.

### Synthesis of C194

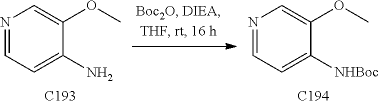

A solution of 3-methoxypyridin-4-amine (16 g, 128.9 mmol) and DIEA (49.97 g, 386.7 mmol) in THF (160 mL) was treated with (Boc)₂O (36.57 g, 167.6 mmol). The resulting mixture was stirred for 16 h at room temperature. The resulting mixture was diluted with water (160 mL). The resulting mixture was extracted with EtOAc (2×200 mL). The combined organic layers were washed with water (2×200 mL) and brine (200 mL), dried over anhydrous Na₂SO₄, and filtered. The filtrate was concentrated under reduced pressure and purified by silica gel column chromatography (50% EtOAc in PE) to afford tert-butyl N-(3-methoxy-4-pyridyl)carbamate (C194, 16 g, 71.4 mmol, 55% yield) as a solid. LCMS (ES, m/z): 225 [M+H]⁺.

### Synthesis of C195

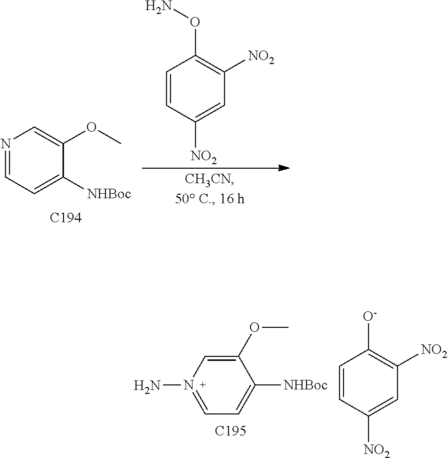

A solution of tert-butyl N-(3-methoxy-4-pyridyl)carbamate (1 g, 4.46 mmol) in MeCN (20 mL) was treated with O-(2,4-dinitrophenyl) hydroxylamine (976 mg, 4.91 mmol), and the reaction was stirred for 16 h at 50° C. under N₂. The mixture was concentrated under reduced pressure to afford 1-amino-4-((tert-butoxycarbonyl)amino)-3-methoxypyridin-1-ium 2,4-dinitrophenolate (C195, 1.5 g, 3.5 mmol, 79% yield) as a solid. LCMS (ES, m/z): 240 [M+H]⁺.

### Synthesis of C196

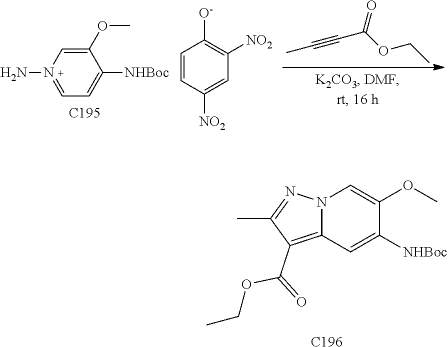

To a stirred mixture of 1-amino-4-((tert-butoxycarbonyl)amino)-3-methoxypyridin-1-ium 2,4-dinitrophenolate (1.5 g, 3.54 mmol) and methyl but-2-ynoate (382 mg, 3.90 mmol) in DMF (20 mL) were added K₂CO₃ (1.47 g, 10.63 mmol), and the reaction was stirred for 16 h at room temperature. The resulting mixture was quenched by the addition of water (60 mL) and extracted with EtOAc (3×50 mL). The combined organic layers were washed with water (2×100 mL) and brine (100 mL), dried over anhydrous Na₂SO₄, and filtered. The filtrate was concentrated under reduced pressure and purified by silica gel column chromatography (25% EtOAc in PE) to afford ethyl 5-(tert-butoxycarbonylamino)-6-methoxy-2-methyl-pyrazolo[1,5-a]pyridine-3-carboxylate (C196, 390 mg, 1.12 mmol, 32% yield) as a solid. LCMS (ES, m/z): 350 [M+H]⁺.

### Synthesis of C197

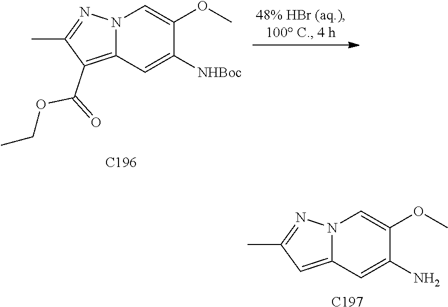

A mixture of ethyl 5-(tert-butoxycarbonylamino)-6-methoxy-2-methyl-pyrazolo[1,5-a]pyridine-3-carboxylate (390 mg, 1.12 mmol) in HBr (48%, aq.) (4 mL) was stirred for 4 h at 100° C. The mixture was basified to pH 8 with Na₂CO₃. The resulting mixture was extracted with EtOAc (3×20 mL). The combined organic layers were washed with brine (50 mL), dried over anhydrous Na₂SO₄, and filtered. The filtrate was concentrated under reduced pressure and purified by silica gel column chromatography (50% EtOAc in PE) to afford 6-methoxy-2-methyl-pyrazolo[1,5-a]pyridin-5-amine (C197, 110 mg, 620.8 μmol, 56% yield) as a solid. LCMS (ES, m/z): 178 [M+H]⁺.

### Example 185: Synthesis of C199

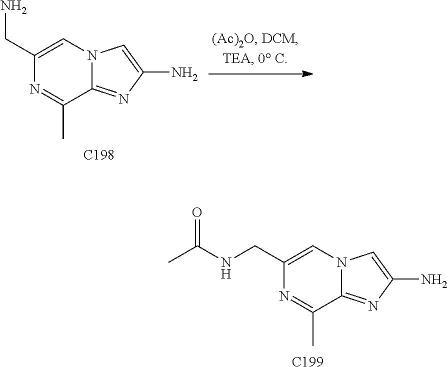 [SMILES: Cc1nc(CN)cn2cc(N)nc12 ; "C198" ; CC(=O)NCc1cn2cc(N)nc2c(C)n1 ; "C199"]

To a stirred mixture of 6-(aminomethyl)-8-methylimidazo[1,2-a]pyrazin-2-amine 2,2,2-trifluoroacetate (150 mg, 515.5 μmol) in DCM (2 mL) was added TEA (256.9 mg, 2.54 mmol) to pH 5 at 0° C. The resulting mixture was added acetyl chloride (66.4 mg, 846.5 μmol), and the reaction was stirred for 2 h at room temperature. The resulting mixture was washed with water (1 mL), dried by anhydrous Na₂SO₄ and concentrated under reduced pressure afford N-[(2-amino-8-methyl-imidazo[1,2-a]pyrazin-6-yl)methyl]acetamide (C199, 120 mg, 547.3 μmol, 65% yield) as a solid. LCMS (ES, m/z): 220 [M+H]⁺.

### Example 186: Synthesis of C208

### Synthesis of C200

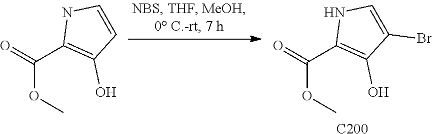

To a stirred mixture of methyl 3-hydroxy-1H-pyrrole-2-carboxylate (11 g, 77.95 mmol) in THF (110 mL) were added MeOH (110 mL) and NBS (27.75 g, 155.9 mmol) at 0° C., and the reaction was stirred for 7 h at room temperature. The resulting mixture was diluted with water (200 mL) and extracted with EtOAc (3×200 mL). The combined organic layers were washed with water (2×500 mL) and brine (500 mL), dried over anhydrous Na₂SO₄, and filtered. The filtrate was concentrated under reduced pressure and purified by silica gel column chromatography (25% EtOAc in PE) to afford methyl 4-bromo-3-hydroxy-1H-pyrrole-2-carboxylate (C200, 8.2 g, 37.3 mmol, 48% yield) as a solid. LCMS (ES, m/z): 220 [M+H]⁺.

### Synthesis of C201

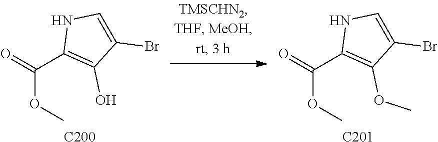

To a stirred mixture of methyl 4-bromo-3-hydroxy-1H-pyrrole-2-carboxylate (7.6 g, 34.54 mmol) in THF (75 mL) were added MeOH (75 mL) and TMSCHN₂ (15.78 g, 138.2 mmol) dropwise at 0° C. under N₂. The resulting mixture was stirred for 3 h at room temperature under N₂. The resulting mixture was quenched with water at 0° C., diluted with water (150 mL), and extracted with EtOAc (3×150 mL). The combined organic layers were washed with water (2×300 mL) and brine (300 mL), dried over anhydrous Na₂SO₄, and filtered. The filtrate was concentrated under reduced pressure and purified by silica gel column chromatography (25% EtOAc in PE) to afford methyl 4-bromo-3-methoxy-1H-pyrrole-2-carboxylate (C201, 6.3 g, 26.9 mmol, 78% yield) as a solid. LCMS (ES, m/z): 234 [M+H]⁺.

### Synthesis of C202

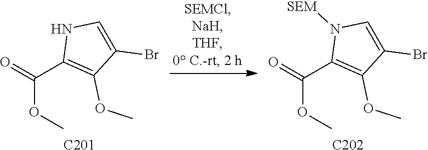 [SMILES: COC(=O)c1[nH]cc(Br)c1OC ; COC(=O)c1c(OC)c(Br)cn1COCC[Si](C)(C)C ; "C201" ; "C202"]

To a solution of methyl 4-bromo-3-methoxy-1H-pyrrole-2-carboxylate (6.3 g, 26.92 mmol) in THF (120 mL) was added NaH (60% dispersion in oil) (1.29 g, 53.84 mmol) at 0° C. The mixture was stirred for 30 min. SEMCl (4.97 g, 29.61 mmol) was added and the mixture was allowed to warm to room temperature and stirred for 2 h. The reaction mixture was quenched by water (100 mL) and extracted with EtOAc (3×150 mL). The combined organic layers were washed with water (2×400 mL) and brine (300 mL), dried over anhydrous Na₂SO₄, and filtered. The filtrate was concentrated under reduced pressure and purified by silica gel column chromatography (25% EtOAc in PE) to afford methyl 4-bromo-3-methoxy-1-(2-trimethylsilylethoxymethyl) pyrrole-2-carboxylate (C202, 6 g, 16.5 mmol, 61% yield) as a solid. LCMS (ES, m/z): 364 [M+H]⁺.

### Synthesis of C203

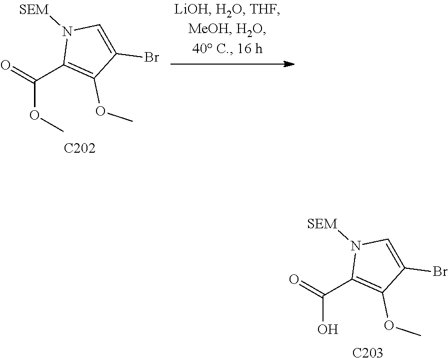 [SMILES: COC(=O)c1c(OC)c(Br)cn1COCC[Si](C)(C)C ; "C202" ; COc1c(Br)cn(COCC[Si](C)(C)C)c1C(=O)O ; "C203"]

To a stirred mixture of methyl 4-bromo-3-methoxy-1-(2-trimethylsilylethoxymethyl) pyrrole-2-carboxylate (6 g, 16.47 mmol) in THF (60 mL) were added H₂O (60 mL) and MeOH (30 mL) and LiOH H₂O (3.46 g, 82.35 mmol), and the reaction was stirred for 16 h at 40° C. The mixture was acidified to pH 5 with HCl (2N) and extracted with EtOAc (3×100 mL). The combined organic layers were washed with water (3×300 mL) and brine (300 mL), dried over anhydrous Na₂SO₄, and filtered. The filtrate was concentrated under reduced pressure to afford 4-bromo-3-methoxy-1-(2-trimethylsilylethoxymethyl) pyrrole-2-carboxylic acid (C203, 5.6 g, 15.99 mmol, 97% yield) as a solid. LCMS (ES, m/z): 350 [M+H]⁺.

### Synthesis of C204

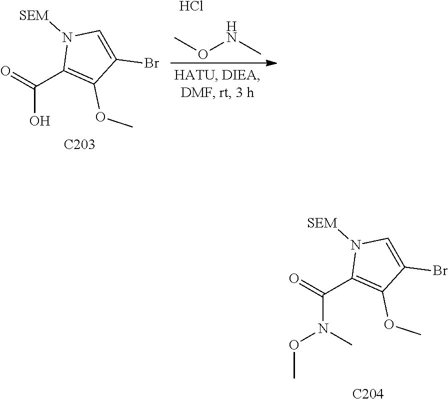

To a stirred mixture of 4-bromo-3-methoxy-1-(2-trimethylsilylethoxymethyl) pyrrole-2-carboxylic acid (5.6 g, 16 mmol) and N-methoxymethanamine (1.46 g, 24 mmol) in DMF (200 mL) were added DIEA (6.20 g, 48 mmol) and HATU (9.12 g, 24 mmol), and the reaction was stirred for 2 h at room temperature. The resulting mixture was diluted with water (600 mL) and extracted with EtOAc (3×300 mL). The combined organic layers were washed with water (3×1 L) and brine (1 μL), dried over anhydrous Na₂SO₄, and filtered. The filtrate was concentrated under reduced pressure and purified by silica gel column chromatography (25% EtOAc in PE) to afford 4-bromo-N,3-dimethoxy-N-methyl-1-(2-trimethylsilylethoxymethyl) pyrrole-2-carboxamide (C204, 4.3 g, 10.9 mmol, 68% yield) as a solid. LCMS (ES, m/z): 393 [M+H]⁺.

### Synthesis of C205

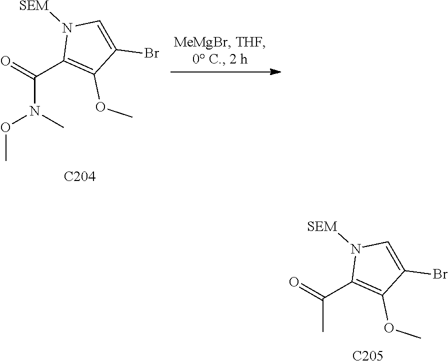 [SMILES: COc1c(Br)cn(COCC[Si](C)(C)C)c1C(=O)N(C)OC ; "C204" ; COc1c(Br)cn(COCC[Si](C)(C)C)c1C(C)=O ; "C205"]

To a stirred mixture of 4-bromo-N,3-dimethoxy-N-methyl-1-(2-trimethylsilylethoxymethyl) pyrrole-2-carboxamide (4.1 g, 10.42 mmol) in THF (80 mL) was added MeMgBr (7 mL, 31.27 mmol) dropwise at 0° C. under N₂. The resulting mixture was stirred for 2 h at 0° C. under N₂. The reaction was quenched with H₂O at 0° C. The resulting mixture was extracted with EtOAc (3×100 mL). The combined organic layers were washed with water (3×300 mL) and brine (300 mL), dried over anhydrous Na₂SO₄, and filtered. The filtrate was concentrated under reduced pressure and purified by silica gel column chromatography (25% EtOAc in PE) to afford 1-[4-bromo-3-methoxy-1-(2-trimethylsilylethoxymethyl) pyrrol-2-yl]ethanone (C205, 3.5 g, 10.1 mmol, 96% yield) as an oil. LCMS (ES, m/z): 348 [M+H]⁺.

### Synthesis of C206

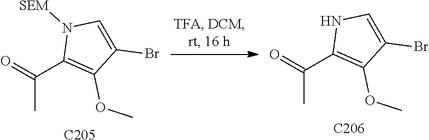 [SMILES: COc1c(Br)cn(COCC[Si](C)(C)C)c1C(C)=O ; COc1c(Br)c[nH]c1C(C)=O ; "C206" ; "C205"]

To a stirred mixture of 1-[4-bromo-3-methoxy-1-(2-trimethylsilylethoxymethyl) pyrrol-2-yl]ethanone (3.2 g, 9.19 mmol) in DCM (60 mL) was added TFA (15 mL) dropwise, and the reaction was stirred for 16 h at room temperature. The resulting mixture was concentrated under reduced pressure. The residue was adjusted to PH 7 with NaHCO₃ (aq.) and extracted with DCM (3×50 mL). The combined organic layer was dried by Na₂SO₄ and then concentrated to afford 1-(4-bromo-3-methoxy-1H-pyrrol-2-yl) ethanone (C206, 1.6 g, 7.34 mmol, 80% yield) as an oil. LCMS (ES, m/z): 218 [M+H]⁺.

### Synthesis of C207

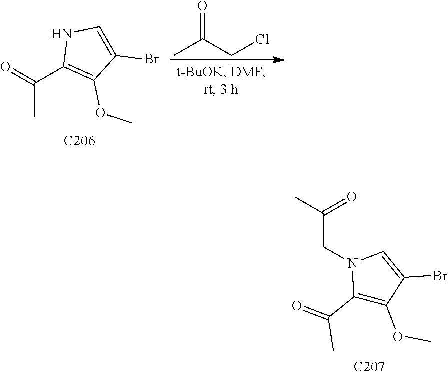

To a stirred mixture of 1-(4-bromo-3-methoxy-1H-pyrrol-2-yl) ethanone (1.1 g, 5.04 mmol) in DMF (20 mL) were added t-BuOK (1.13 g, 10.09 mmol) and 1-chloropropan-2-one (933.5 mg, 10.09 mmol) at 0° C., and the reaction was stirred for 3 h at room temperature. The reaction was quenched with water at 0° C., diluted with water (60 mL), and extracted with EtOAc (3×60 mL). The combined organic layers were washed with water (3×180 mL) and brine (180 mL), dried over anhydrous Na₂SO₄, and filtered. The filtrate was concentrated under reduced pressure and purified by silica gel column chromatography (50% EtOAc in PE) to afford 1-(2-acetyl-4-bromo-3-methoxy-pyrrol-1-yl)propan-2-one (C207, 310 mg, 1.13 mmol, 22% yield) as an oil. LCMS (ES, m/z): 274 [M+H]⁺.

### Synthesis of C208

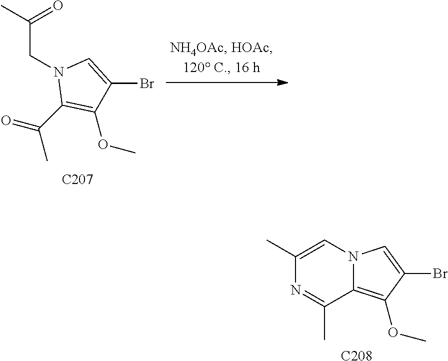

To a stirred mixture of 1-acetonyl-4-bromo-3-methoxy-pyrrole-2-carbaldehyde (400 mg, 1.54 mmol) in HOAc (8 mL) was added NH₄OAc (2.37 g, 30.76 mmol), and the reaction was stirred for 16 h at 120° C. The resulting mixture was concentrated under reduced pressure and purified by silica gel column chromatography (50% EtOAc in PE) to afford 7-bromo-8-methoxy-1,3-dimethyl-pyrrolo[1,2-a]pyrazine (C208, 200 mg, 784 μmol, 51% yield) as an oil. LCMS (ES, m/z): 255 [M+H]⁺.

### Example 187: Synthesis of C211

### Synthesis of C210

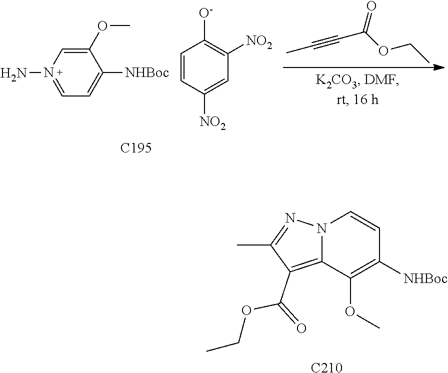

To a stirred mixture of 1-amino-4-((tert-butoxycarbonyl)amino)-3-methoxypyridin-1-ium 2,4-dinitrophenolate (1.5 g, 3.54 mmol) and ethyl but-2-ynoate (382 mg, 3.90 mmol) in DMF (20 mL) were added K₂CO₃ (1.47 g, 10.63 mmol), and the reaction was stirred for 16 h at room temperature. The resulting mixture was quenched by the addition of H₂O (60 mL) and extracted with EtOAc (3×50 mL). The combined organic layers were washed with water (2×100 mL) and brine (100 mL), dried over anhydrous Na₂SO₄, and filtered. The filtrate was concentrated under reduced pressure and purified by silica gel column chromatography (17% EtOAc in PE) to afford ethyl 5-((tert-butoxycarbonyl)amino)-4-methoxy-2-methylpyrazolo[1,5-a]pyridine-3-carboxylate (C210, 410 mg, 1.12 mmol, 34% yield) as a solid. LCMS (ES, m/z): 350 [M+H]⁺.

### Synthesis of C211

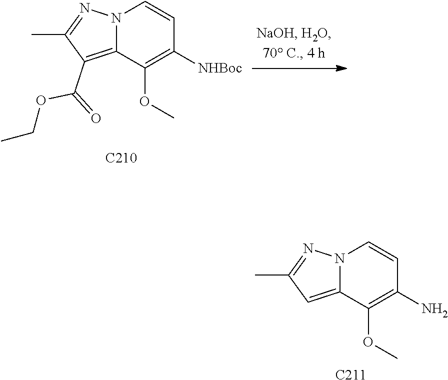 [SMILES: CCOC(=O)c1c(C)nn2ccc(NC(=O)OC(C)(C)C)c(OC)c12 ; "C210" ; COc1c(N)ccn2nc(C)cc12 ; "C211"]

A solution of ethyl 5-(tert-butoxycarbonylamino)-4-methoxy-2-methyl-pyrazolo[1,5-a]-pyridine-3-carboxylate (340 mg, 973.2 μmol) in H₂O (3 mL) was treated with NaOH (155 mg, 3.89 mmol), and the reaction was stirred for 4 h at 70° C. The mixture was acidified to pH 5 with HCl (1N) and extracted with EtOAc (3×20 mL). The combined organic layers were washed with brine (50 mL), dried over anhydrous Na₂SO₄, and filtered. The filtrate was concentrated under reduced pressure, to afford 4-methoxy-2-methyl-pyrazolo[1,5-a]pyridin-5-amine (C211, 90 mg, 507.9 μmol, 52% yield) as a solid. LCMS (ES, m/z): 178 [M+H]⁺.

### Example 188: Synthesis of C214

### Synthesis of C212

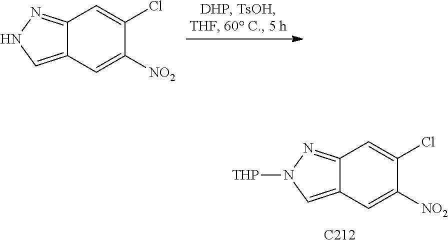

To a stirred mixture of 6-chloro-5-nitro-2H-indazole (2.1 g, 10.63 mmol) in THF (30 mL) was added TsOH (2.01 g, 11.69 mmol) and DHP (1.34 g, 15.94 mmol), and the reaction was stirred for 5 h at 50° C. under N₂. The resulting mixture was diluted with water (30 mL) and extracted with EtOAc (3×30 mL). The combined organic layers were washed with brine (2×60 mL), dried over anhydrous Na₂SO₄, and filtered. The filtrate was concentrated under reduced pressure and purified by silica gel column chromatography (50% EtOAc in PE) to afford 6-chloro-5-nitro-2-tetrahydropyran-2-yl-indazole (C212, 2.8 g, 9.94 mmol, 94% yield) as a solid. LCMS (ES, m/z): 282 [M+H]⁺.

### Synthesis of C213

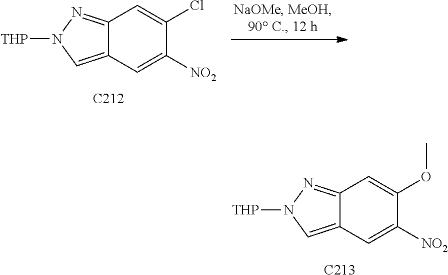

To a stirred mixture of 6-chloro-5-nitro-2-tetrahydropyran-2-yl-indazole (0.5 g, 1.77 mmol) in MeOH (10 mL) was added NaOMe (191.7 mg, 3.55 mmol) dropwise, and the reaction was stirred for 12 h at 90° C. under N₂. The resulting mixture was diluted with water (10 mL) and extracted with EtOAc (3×10 mL). The combined organic layers were washed with brine (2×10 mL), dried over anhydrous Na₂SO₄, and filtered. The filtrate was concentrated under reduced pressure and purified by silica gel column chromatography (67% EtOAc in PE) to afford 6-methoxy-5-nitro-2-tetrahydropyran-2-yl-indazole (C213, 0.3 g, 1.08 mmol, 61% yield) as a solid. LCMS (ES, m/z): 278 [M+H]⁺.

### Synthesis of C214

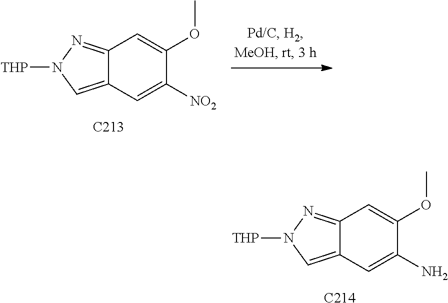

To a solution of 6-methoxy-5-nitro-2-tetrahydropyran-2-yl-indazole (0.2 g, 721.3 μmol) in 5 mL of MeOH was added Pd/C (10%, 230 mg) under N₂. The mixture was hydrogenated at room temperature for 3 h under hydrogen atmosphere. The mixture was filtered and the solid was washed with MeOH (10 mL). The filtrate was concentrated under reduced pressure to afford 6-methoxy-2-tetrahydropyran-2-yl-indazol-5-amine (C214, 0.15 g, 606.6 μmol, 84% yield) as an oil. LCMS (ES, m/z): 248 [M+H]⁺.

### Example 189: Synthesis of C217

### Synthesis of C215

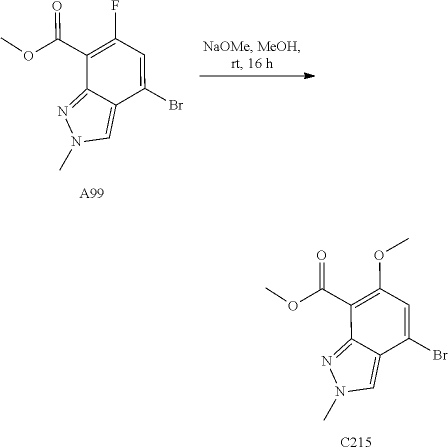

To a solution of methyl 4-bromo-6-fluoro-2-methyl-indazole-7-carboxylate (A99, 800 mg, 2.79 mmol) in MeOH (8 mL) was added NaOMe (30 wt. % solution in MeOH) (301.1 mg, 5.57 mmol), and the reaction was stirred for 16 h at rt. The resulting mixture was quenched with H₂O at 0° C., diluted with H₂O (30 mL), and extracted with DCM (2×30 mL). The combined organic layers were washed with H₂O (2×30 mL), dried over anhydrous Na₂SO₄, and filtered. The filtrate was concentrated under reduced pressure, to afford methyl 4-bromo-6-methoxy-2-methyl-indazole-7-carboxylate (C215, 800 mg, 2.7 mmol, 96% yield) as an oil. LCMS (ES, m/z): 299 [M+H]⁺.

### Synthesis of C216

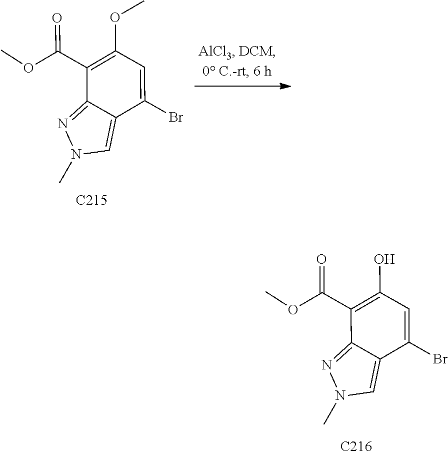

To a solution of methyl 4-bromo-6-methoxy-2-methyl-indazole-7-carboxylate (700 mg, 2.34 mmol) in DCM (7 mL) was added AlCl₃ (936.1 mg, 7.02 mmol) at 0° C. under N₂. The mixture was stirred for 6 h at rt. The reaction was quenched with H₂O at 0° C. The reaction mixture was filtered to remove insoluble solid, the filter cake was washed with DCM. The organic layer was separated and the aqueous layer was extracted with DCM (2×20 mL). All of the organic layers were combined and washed with H₂O (2×20 mL), dried over anhydrous Na₂SO₄, and filtered. The filtrate was concentrated under reduced pressure, to afford methyl 4-bromo-6-hydroxy-2-methyl-indazole-7-carboxylate (C216, 200 mg, 701.5 μmol, 30% yield) as an oil. LCMS (ES, m/z): 285 [M+H]⁺.

### Synthesis of C217

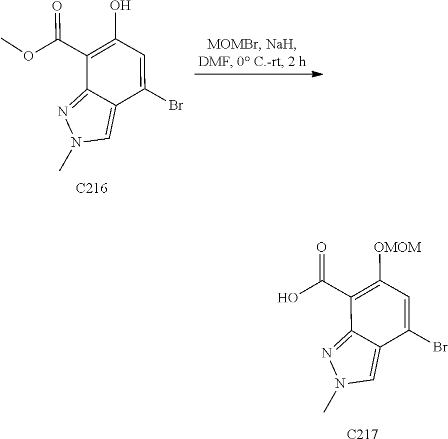

To a solution of methyl 4-bromo-6-hydroxy-2-methyl-indazole-7-carboxylate (160 mg, 561.2 μmol) in DMF (2 mL) was added NaH (60% dispersion in oil) (20.2 mg, 841.8 μmol) at 0° C. under N₂. The mixture was stirred for 1 h at 0° C. Then bromo(methoxy) methane (84.2 mg, 673.5 μmol) was added at 0° C. The mixture was stirred for 2 h at rt. The reaction was quenched with H₂O at 0° C. The resulting mixture was extracted with DCM (2×10 mL). The combined organic layers were washed with H₂O (2×15 mL), dried over anhydrous Na₂SO₄, and filtered. The filtrate was concentrated under reduced pressure and purified by silica gel column chromatography (67% EtOAc in PE) to afford 4-bromo-6-(methoxymethoxy)-2-methyl-indazole-7-carboxylic acid (C217, 160 mg, 507.7 μmol, 90% yield) as an oil. LCMS (ES, m/z): 315 [M+H]⁻.

### Example 190: Synthesis of Compound 463

### Synthesis of B177

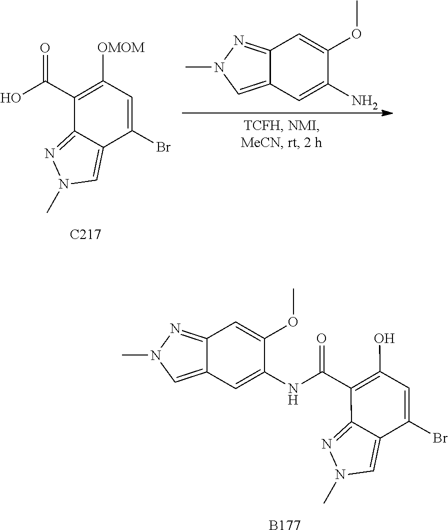

To a solution of 4-bromo-6-(methoxymethoxy)-2-methyl-indazole-7-carboxylic acid (140 mg, 444.3 μmol) in MeCN (1.5 mL) were added 6-methoxy-2-methyl-indazol-5-amine (86.6 mg, 488.7 μmol), NMI (218.8 mg, 2.67 mmol) and TCFH (124.6 mg, 444.3 μmol), and the reaction was stirred for 2 h at rt. The reaction was diluted with H₂O and the precipitated solids were collected by filtration and purified by trituration with MTBE to afford 4-bromo-6-hydroxy-N-(6-methoxy-2-methyl-indazol-5-yl)-2-methyl-indazole-7-carboxamide (B177, 95 mg, 220.8 μmol, 50% yield) as a solid. LCMS (ES, m/z): 430 [M+H]⁺.

### Synthesis of B178

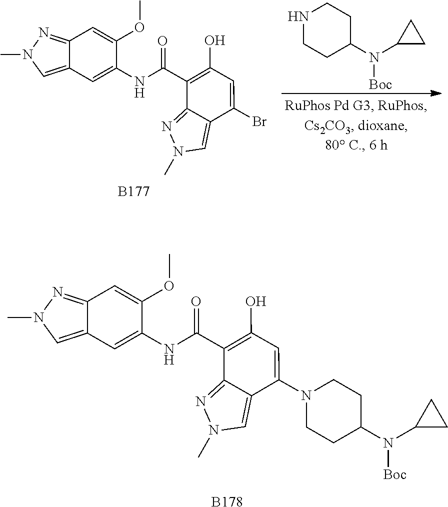

To a stirred mixture of 4-bromo-6-hydroxy-N-(6-methoxy-2-methyl-indazol-5-yl)-2-methyl-indazole-7-carboxamide (90 mg, 209.2 μmol) in dioxane (1 mL) were added tert-butyl N-cyclopropyl-N-(4-piperidyl)carbamate (55.3 mg, 230.1 μmol), Cs₂CO₃ (136.3 mg, 418.4 μmol), Ruphos (19.5 mg, 41.84 μmol) and RuPhos Pd G3 (17.5 mg, 20.92 μmol), and the reaction was stirred for 6 h at 80° C. under N₂. The resulting mixture was diluted with H₂O (15 mL) and extracted with EtOAc (2×15 mL). The combined organic layers were washed with H₂O (2×15 mL), dried over anhydrous Na₂SO₄, and filtered. The filtrate was concentrated under reduced pressure and purified by silica gel column chromatography (17% EtOAc in PE) to afford tert-butyl N-cyclopropyl-N-[1-[6-hydroxy-7-[(6-methoxy-2-methyl-indazol-5-yl)carbamoyl]-2-methyl-indazol-4-yl]-4-piperidyl]carbamate (B178, 40 mg, 67.8 μmol, 32% yield) as an oil. LCMS (ES, m/z): 590 [M+H]⁺.

### Synthesis of Compound 463

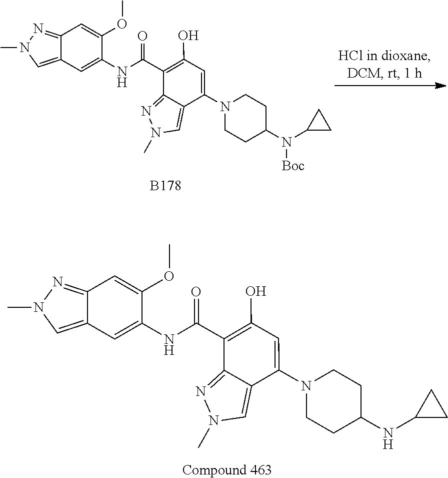

To a solution of tert-butyl N-cyclopropyl-N-[1-[6-hydroxy-7-[(6-methoxy-2-methyl-indazol-5-yl)carbamoyl]-2-methyl-indazol-4-yl]-4-piperidyl]carbamate (37 mg, 62.75 μmol) in DCM (0.4 mL) was added HCl (4.0 M in 1,4-dioxane) (0.15 mL), and the reaction was stirred for 2 h at rt. The resulting mixture was concentrated under reduced pressure and purified by Prep-HPLC (Condition 5, Gradient 8) to afford 4-[4-(cyclopropylamino)-1-piperidyl]-6-hydroxy-N-(6-methoxy-2-methyl-indazol-5-yl)-2-methyl-indazole-7-carboxamide (Compound 463, 5 mg, 10.2 μmol, 16% yield) as a solid. LCMS (ES, m/z): 490 [M+H]⁺. ¹H NMR (400 MHz, DMSO-d₆) δ 13.89 (s, 1H), 11.64 (s, 1H), 8.65 (s, 1H), 8.58 (s, 1H), 8.18 (s, 1H), 7.06 (s, 1H), 5.90 (s, 1H), 4.16 (s, 3H), 4.07 (s, 3H), 4.02 (s, 3H), 3.84 (d, J=13.1 Hz, 2H), 3.03 (t, J=11.7 Hz, 2H), 2.75 (t, J=9.3 Hz, 1H), 2.24 (s, 1H), 2.10 (tt, J=6.7, 3.6 Hz, 1H), 1.96 (d, J=12.7 Hz, 2H), 1.42 (q, J=9.8 Hz, 2H), 0.38 (td, J=6.3, 4.0 Hz, 2H), 0.25-0.17 (m, 2H).

An analogous method was followed to obtain the following compounds.

| | Starting | |
|---|---|---|
| Compound | Material | Characterization |
| 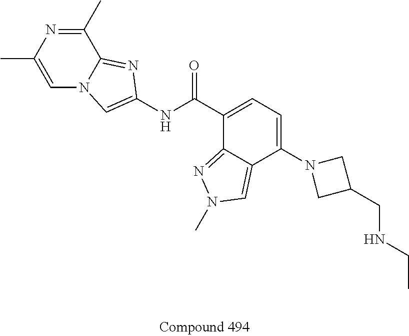 [SMILES: CCNCC1CN(c2ccc(C(=O)Nc3cn4cc(C)nc(C)c4n3)c3nn(C)cc23)C1] | 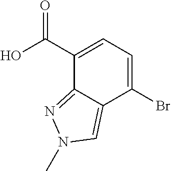 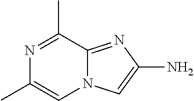 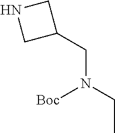 | Modification: Step 1 was ran for 3 h. Step 2 was ran at 90° C. Step 3 was ran using TFA in DCM for 1 h at room temperature. The residue was purified by reversed- phase flash chromatography (Condition 3, Gradient 20) to afford N-(6,8- dimethylimidazo[1,2-a]-pyrazin-2-yl)-4- [3-(ethylaminomethyl)azetidin-1-yl]-2- methyl-indazole-7-carboxamide (Compound 494, 18.4 mg, 42.6 μmol). LCMS (ES, m/z): 433 [M + H]⁺. ¹H NMR (300 MHz, DMSO-d₆) δ 11.29 (s, 1H), 8.68 (s, 1H), 8.30 (d, J = 2.5 Hz, 2H), 7.97 (d, J = 8.1 Hz, 1H), 5.88 (d, J = 8.1 Hz, 1H), 4.32 (d, J = 8.2 Hz, 2H), 4.27 (s, 3H), 4.00-3.89 (m, 2H), 2.91 (s, 1H), 2.81 (d, J = 7.1 Hz, 2H), 2.69 (s, 3H), 2.57 (q, J = 7.1 Hz, 2H), 2.37 (s, 3H), 1.03 (t, J = 7.1 Hz, 3H). |

### Example 191: Synthesis of Compound 466

### Synthesis of B179

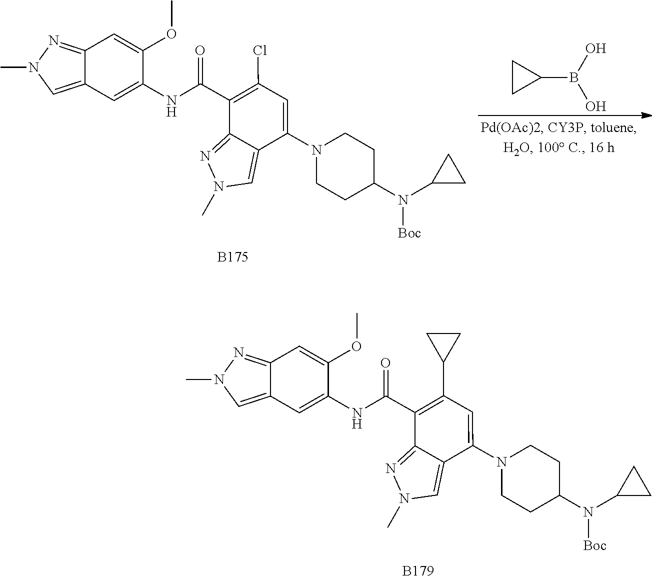

To a solution of tert-butyl N-[1-[6-chloro-7-[(6-methoxy-2-methyl-indazol-5-yl)carbamoyl]-2-methyl-indazol-4-yl]-4-piperidyl]-N-cyclopropyl-carbamate (200.0 mg, 328.9 μmol) and cyclopropylboronic acid (197.7 mg, 2.30 mmol) in toluene (4 mL) and H₂O (1 mL) were added K₂CO₃ (90.9 mg, 657.8 μmol) and Cy₃P (20.6 mg, 65.78 μmol), Pd(OAc)₂ (7.3 mg, 32.89 μmol), and the reaction was stirred for 16 h at 100° C. under N₂. The resulting mixture was concentrated under reduced pressure and purified by silica gel column chromatography (5% MeOH in DCM) to afford tert-butyl N-cyclopropyl-N-[1-[6-cyclopropyl-7-[(6-methoxy-2-methyl-indazol-5-yl)carbamoyl]-2-methyl-indazol-4-yl]-4-piperidyl]carbamate (B179, 120.0 mg, 195.5 μmol, 59% yield) as a solid. LCMS (ES, m/z): 614 [M+H]⁺.

### Synthesis of Compound 466

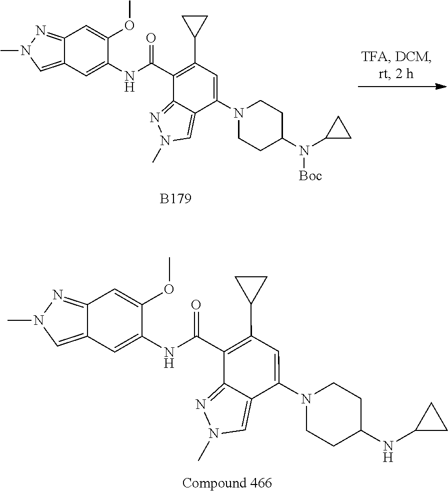

To a solution of tert-butyl N-cyclopropyl-N-[1-[6-cyclopropyl-7-[(6-methoxy-2-methyl-indazol-5-yl)carbamoyl]-2-methyl-indazol-4-yl]-4-piperidyl]carbamate (100 mg, 162.9 μmol) in DCM (1 mL) was added TFA (0.4 mL). The reaction was stirred for 2 h at rt. The resulting mixture was concentrated under reduced pressure and adjusted to pH 8 with saturated NaHCO₃ (aq.). The resulting mixture was purified by reversed-phase flash chromatography (Condition 3, Gradient 26) to afford 6-cyclopropyl-4-[4-(cyclopropylamino)-1-piperidyl]-N-(6-methoxy-2-methyl-indazol-5-yl)-2-methyl-indazole-7-carboxamide (Compound 466, 41 mg, 79.8 μmol, 49% yield) as a solid. LCMS (ES, m/z): 614 [M+H]⁺. ¹H NMR (300 MHz, DMSO-d₆) δ 11.49 (s, 1H), 8.82 (s, 1H), 8.58 (s, 1H), 8.16 (s, 1H), 7.05 (s, 1H), 5.82 (s, 1H), 4.22 (s, 3H), 4.09 (s, 3H), 4.00 (s, 3H), 3.77 (d, J=12.4 Hz, 2H), 3.58 (d, J=5.1 Hz, 1H), 2.93 (t, J=11.7 Hz, 2H), 2.74 (s, 1H), 2.11 (d, J=17.0 Hz, 1H), 2.00 (d, J=12.6 Hz, 2H), 1.47 (q, J=10.6, 10.2 Hz, 2H), 0.99 (d, J=8.6 Hz, 2H), 0.79 (d, J=4.9 Hz, 2H), 0.45-0.36 (m, 2H), 0.25 (d, J=3.9 Hz, 2H).

An analogous method was followed to obtain the following compounds.

| Compound | Starting Material | Characterization |
|---|---|---|
| 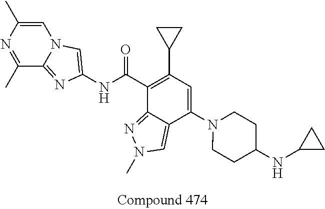 [SMILES: Cc1cn2cc(NC(=O)c3c(C4CC4)cc(N4CCC(NC5CC5)CC4)c4cn(C)nc34)nc2c(C)n1] | 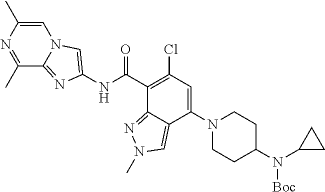 | Modification: Step 1 was ran for 72 h at 110° C. The residue was purified by reversed-phase flash chromatography (Condition 3, Gradient) to afford 6-cyclopropyl- 4-[4-(cyclopropylamino)-1- piperidyl]-N-(6,8-dimethyl- imidazo[1,2-a]-pyrazin-2-yl)-2- methyl-indazole-7-carboxamide (Compound 474, 5 mg, 10.0 μmol). LCMS (ES, m/z): 499 [M + H]⁺. ¹H NMR (400 MHz, DMSO-d₆) δ 11.77 (s, |
| | | 1H), 8.58 (s, 1H), 8.34 (s, 1H), |
| | | 8.29 (s, 1H), 5.84 (s, 1H), 4.20 (s, |
| | | 3H), 3.77 (d, J = 12.4 Hz, 2H), |
| | | 3.39-3.34 (s, 1H), 2.94 (t, J = 11.5 |
| | | Hz, 2H), 2.75 (d, J = 11.5 Hz, |
| | | 1H), 2.69 (s, 3H), 2.38 (s, 3H), |
| | | 2.12 (dq, J = 6.7, 3.4 Hz, 1H), |
| | | 1.99 (d, J = 12.6 Hz, 2H), 1.46 |
| | | (q, J = 11.1 Hz, 2H), 1.02-0.93 |
| | | (m, 2H), 0.81 (dt, J = 5.6, 2.9 Hz, |
| | | 2H), 0.40 (dt, J = 6.1, 3.0 Hz, 2H), |
| | | 0.27-0.19 (m, 2H). |

### Example 192: Synthesis of C220

### Synthesis of C218

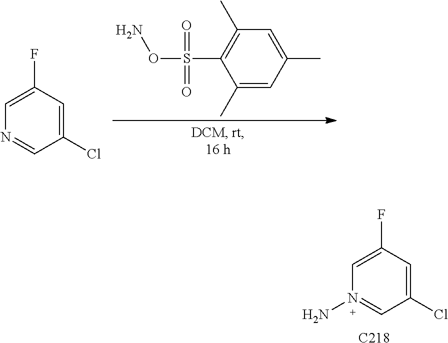

To a stirred mixture of 3-chloro-5-fluoro-pyridine (8.0 g, 60.82 mmol) in DCM (150 mL) was added amino 2,4,6-trimethylbenzenesulfonate (15.71 g, 72.98 mmol), and the reaction was stirred for 16 h at room temperature. The resulting mixture was monitored by LCMS. The precipitated solids were collected by filtration and washed with DCM (20 mL), to afford 3-chloro-5-fluoro-pyridin-1-ium-1-amine (C218, 8.5 g, 57.6 mmol, 95% yield) as a solid. LCMS (ES, m/z): 147 [M]⁺.

### Synthesis of C219

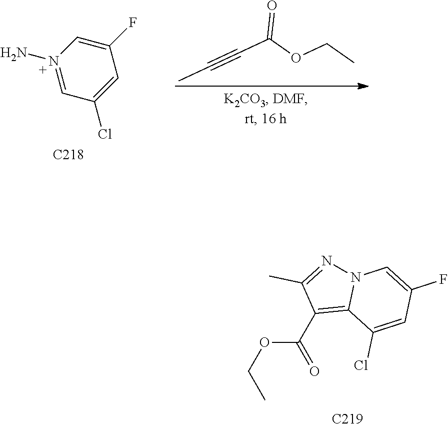

To a stirred mixture of 3-chloro-5-fluoro-pyridin-1-ium-1-amine (8 g, 54.22 mmol) and ethyl but-2-ynoate (12.16 g, 108.4 mmol, 12.57 mL) in DMF (150 mL) was added K₂CO₃ (14.99 g, 108.4 mmol), and the reaction was stirred for 16 h at room temperature. The resulting mixture was monitored by LCMS. The reaction was quenched with water (100 mL) and extracted with EtOAc (2×100 mL). The combined organic layers were washed with brine (100 mL), dried over anhydrous Na₂SO₄, and filtered. The filtrate was concentrated under reduced pressure and purified by silica gel column chromatography (20% EtOAc in PE) to afford ethyl 4-chloro-6-fluoro-2-methyl-pyrazolo[1,5-a]pyridine-3-carboxylate (C219, 0.53 g, 2.06 mmol, 4% yield) as a solid. LCMS (ES, m/z): 257 [M+H]⁺.

### Synthesis of C220

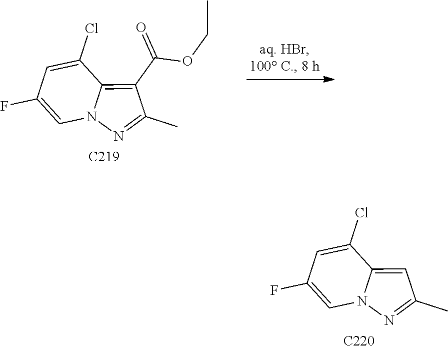 [SMILES: CCOC(=O)c1c(C)nn2cc(F)cc(Cl)c12 ; "C219" ; Cc1cc2c(Cl)cc(F)cn2n1 ; "C220"]

A solution of ethyl 4-chloro-6-fluoro-2-methyl-pyrazolo[1,5-a]pyridine-3-carboxylate (520 mg, 2.03 mmol) in HBr (48% in water) (7 mL) was stirred for 8 h at 100° C. The resulting mixture was diluted with water (50 mL) and extracted with EtOAc (2×50 mL). The combined organic layers were washed with brine (50 mL), dried over anhydrous Na₂SO₄, and filtered. The filtrate was concentrated under reduced pressure to afford 4-chloro-6-fluoro-2-methylpyrazolo-[1,5-a]pyridine (C220, 350 mg, 1.9 mmol, 94% yield) as a solid. LCMS (ES, m/z): 185 [M+H]⁺.

### Synthesis of B180

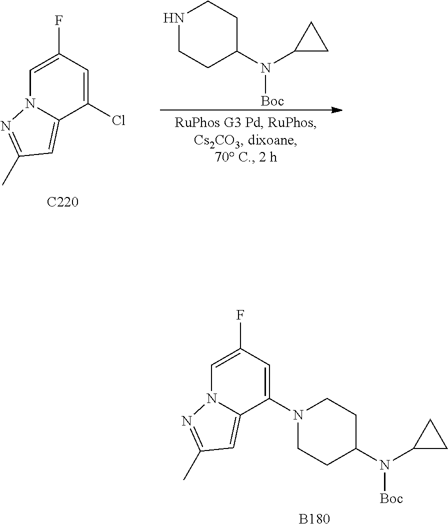

To a solution of 4-chloro-6-fluoro-2-methyl-pyrazolo[1,5-a]pyridine (370.0 mg, 2.0 mmol) and tert-butyl N-cyclopropyl-N-(4-piperidyl)carbamate (529.9 mg, 2.20 mmol) in dioxane (7 mL) were added Cs₂CO₃ (1.31 g, 4.01 mmol) and RuPhos (187.0 mg, 400.9 μmol), RuPhos Pd G3 (167.6 mg, 200.4 μmol), and the reaction was stirred for 2 h at 70° C. under N₂. The resulting mixture was concentrated under reduced pressure and purified by silica gel column chromatography (20% EtOAc in PE) to afford tert-butyl N-cyclopropyl-N-[1-(6-fluoro-2-methyl-pyrazolo[1,5-a]-pyridin-4-yl)-4-piperidyl]carbamate (B180, 580 mg, 1.5 mmol, 74% yield) as a solid. LCMS (ES, m/z): 389 [M+H]⁺.

### Synthesis of B181

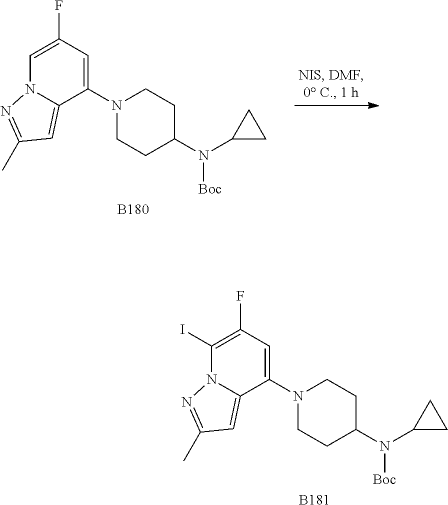

To a stirred mixture of tert-butyl N-cyclopropyl-N-[1-(6-fluoro-2-methyl-pyrazolo[1,5-a]pyridin-4-yl)-4-piperidyl]carbamate (520.0 mg, 1.34 mmol) in DMF (10 mL) was added NIS (301.1 mg, 1.34 mmol) at 0° C. under N₂. The resulting mixture was stirred for 1 h at 0° C. under N₂. The reaction was monitored by LCMS. The reaction was quenched with water (50 mL) at rt. The resulting mixture was extracted with EtOAc (2×50 mL). The combined organic layers were washed with brine (50 mL), dried over anhydrous Na₂SO₄, and filtered. The filtrate was concentrated under reduced pressure and purified by silica gel column chromatography (20% EtOAc in PE) to afford tert-butyl N-cyclopropyl-N-[1-(6-fluoro-7-iodo-2-methyl-pyrazolo[1,5-a]pyridin-4-yl)-4-piperidyl]carbamate (B181, 600 mg, 1.17 mmol, 87% yield) as a solid. LCMS (ES, m/z): 515 [M+H]⁺.

### Synthesis of B182

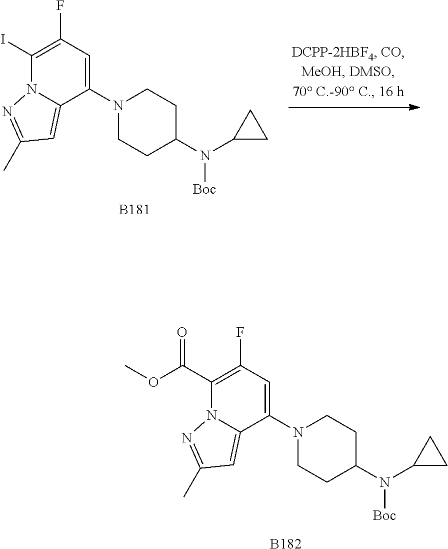 [SMILES: "B181" ; "B182" ; Cc1cc2c(N3CCC(N(C(=O)OC(C)(C)C)C4CC4)CC3)cc(F)c(I)n2n1 ; COC(=O)c1c(F)cc(N2CCC(N(C(=O)OC(C)(C)C)C3CC3)CC2)c2cc(C)nn12]

To a solution of tert-butyl N-cyclopropyl-N-[1-(6-fluoro-7-iodo-2-methyl-pyrazolo[1,5-a]pyridin-4-yl)-4-piperidyl]carbamate (300 mg, 583.2 μmol) in DMSO (3 mL) was added DCPP-2HBF₄ (122.2 mg, 233.3 μmol) in a pressure tank. The mixture was purged with nitrogen for 1 min and then was pressurized to 2 MPa with carbon monoxide at 90° C. for 16 h. The reaction mixture was cooled to room temperature and filtered to remove insoluble solids. The resulting mixture was diluted with water (20 mL) and extracted with EtOAc (2×20 mL). The combined organic layers were washed with brine (20 mL), dried over anhydrous Na₂SO₄, and filtered. The filtrate was concentrated under reduced pressure and purified by silica gel column chromatography (25% EtOAc in PE) to afford methyl 4-[4-[tert-butoxycarbonyl(cyclopropyl)amino]-1-piperidyl]-6-fluoro-2-methyl-pyrazolo[1,5-a]pyridine-7-carboxylate (B182, 120 mg, 268.8 μmol, 46% yield) as a solid. LCMS (ES, m/z): 447 [M+H]⁺.

### Synthesis of B183

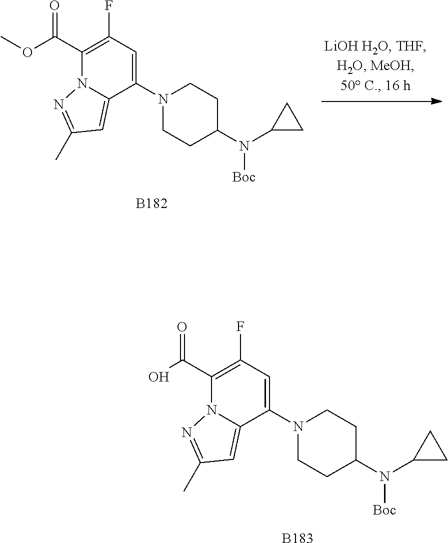 [SMILES: "B182" ; "B183" ; COC(=O)c1c(F)cc(N2CCC(N(C(=O)OC(C)(C)C)C3CC3)CC2)c2cc(C)nn12 ; Cc1cc2c(N3CCC(N(C(=O)OC(C)(C)C)C4CC4)CC3)cc(F)c(C(=O)O)n2n1]

To a stirred solution of methyl 4-[4-[tert-butoxycarbonyl(cyclopropyl)amino]-1-piperidyl]-6-fluoro-2-methyl-pyrazolo[1,5-a]pyridine-7-carboxylate (120.0 mg, 268.8 μmol) in THF (1 mL) and MeOH (1 mL) was added LiOH·H₂O (67.6 mg, 1.61 mmol), and the reaction was stirred for 16 h at 50° C. The resulting mixture was diluted with water (5 mL), acidified to pH 6 with HCl (2 N), and extracted with EtOAc (2×5 mL). The combined organic layers were washed with brine (5 mL), dried over anhydrous Na₂SO₄, and filtered. The filtrate was concentrated under reduced pressure, to afford 4-[4-[tert-butoxycarbonyl(cyclopropyl)amino]-1-piperidyl]-6-fluoro-2-methyl-pyrazolo[1,5-a]pyridine-7-carboxylic acid (B183, 105 mg, 242.8 μmol, 90% yield) as a solid. LCMS (ES, m/z): 433 [M+H]⁺.

### Synthesis of B184

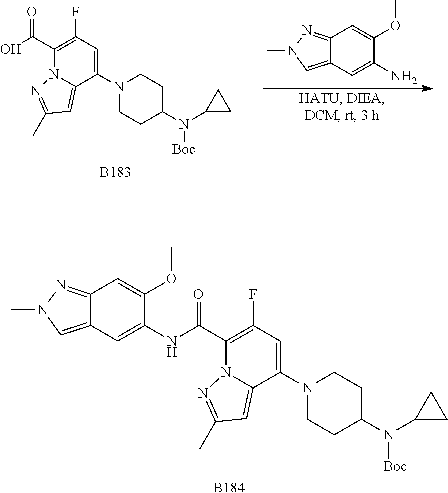

To a stirred mixture of 4-[4-[tert-butoxycarbonyl(cyclopropyl)amino]-1-piperidyl]-6-fluoro-2-methyl-pyrazolo[1,5-a]pyridine-7-carboxylic acid (50.0 mg, 115.6 μmol) and 6-methoxy-2-methyl-indazol-5-amine (24.5 mg, 138.7 μmol) in DCM (1 mL) was added HATU (74.7 mg, 196.5 μmol) and DIEA (74.7 mg, 578.1 μmol, 100.7 μL), and the reaction was stirred for 2 h at room temperature. The reaction was monitored by LCMS. The reaction was quenched with water (5 mL) and extracted with DCM (2×5 mL). The combined organic layers were washed with brine (5 mL), dried over anhydrous Na₂SO₄, and filtered. The filtrate was concentrated under reduced pressure and purified by silica gel column chromatography (50% EtOAc in PE) to afford tert-butyl N-cyclopropyl-N-[1-[6-fluoro-7-[(6-methoxy-2-methyl-indazol-5-yl)carbamoyl]-2-methyl-pyrazolo[1,5-a]pyridin-4-yl]-4-piperidyl]carbamate (B184, 50 mg, 84.5 μmol, 73% yield) as a solid. LCMS (ES, m/z): 592 [M+H]⁺.

### Synthesis of Compound 624

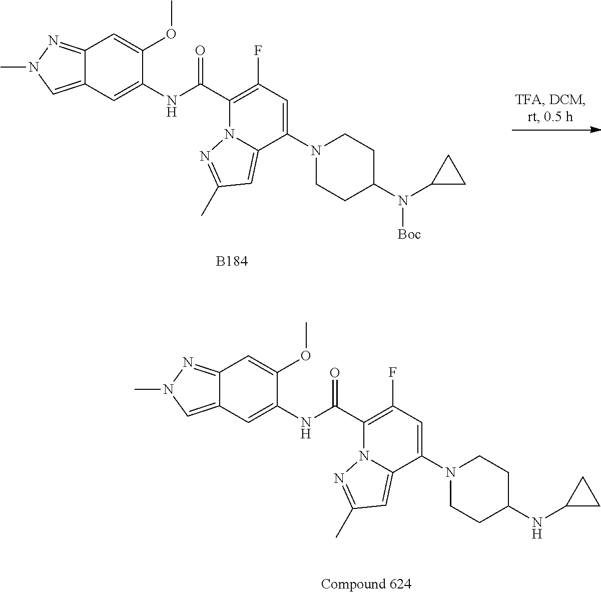 [SMILES: "B184" ; COc1cc2nn(C)cc2cc1NC(=O)c1c(F)cc(N2CCC(N(C(=O)OC(C)(C)C)C3CC3)CC2)c2cc(C)nn12 ; COc1cc2nn(C)cc2cc1NC(=O)c1c(F)cc(N2CCC(NC3CC3)CC2)c2cc(C)nn12 ; "Compound 624"]

To a solution of tert-butyl N-cyclopropyl-N-[1-[6-fluoro-7-[(6-methoxy-2-methyl-indazol-5-yl)carbamoyl]-2-methyl-pyrazolo[1,5-a]pyridin-4-yl]-4-piperidyl]carbamate (45 mg, 76.06 μmol) in DCM (1 mL) was added TFA (0.4 mL). The reaction was stirred for 1 h at rt. The resulting mixture was concentrated under reduced pressure and adjusted to pH 8 with saturated NaHCO₃ (aq.). The resulting mixture was diluted with water (5 mL) and extracted with DCM/MeOH (20/1) (2×10 mL). The combined organic layers were washed with water (20 mL), brine (20 mL) and dried over anhydrous Na₂SO₄, and filtered. The filtrate was concentrated under reduced pressure and purified by reversed-phase flash chromatography (Condition 3, Gradient 31) to afford 4-[4-(cyclopropylamino)-1-piperidyl]-6-fluoro-N-(6-methoxy-2-methyl-indazol-5-yl)-2-methyl-pyrazolo[1,5-a]pyridine-7-carboxamide (Compound 624, 11 mg, 22.4 μmol, 29% yield) as a solid. LCMS (ES, m/z): 492 [M+H]⁻. ¹H NMR (400 MHz, DMSO-d₆) δ 9.57 (s, 1H), 8.73 (dd, J=3.8, 2.0 Hz, 1H), 8.65 (s, 1H), 8.20 (s, 1H), 7.18 (dd, J=10.8, 2.1 Hz, 1H), 7.03 (s, 1H), 4.09 (s, 3H), 3.84 (s, 3H), 3.29 (s, 2H), 2.73 (t, J=11.1 Hz, 2H), 2.54 (s, 3H), 1.83-1.68 (m, 4H), 1.30 (d, J=9.7 Hz, 2H), 0.22-0.13 (m, 2H), −0.01 (d, J=3.0 Hz, 2H).

An analogous method was followed to obtain the following compounds.

| | Starting | |
|---|---|---|
| Compound | Material | Characterization |
| 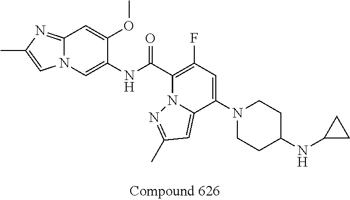 [SMILES: COc1cc2nc(C)cn2cc1NC(=O)c1c(F)cc(N2CCC(NC3CC3)CC2)c2cc(C)nn12] | 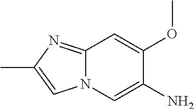 | Modification: Step 8 was ran for 16 h. The residue was purified by reversed-phase flash chroma- tography (Condition 3, Gradient 18) to afford 4-[4- (cyclopropylamino)-1-piperidyl]-6-fluoro-N-(7- methoxy-2-methylimidazo-[1,2-a]pyridin-6-yl)-2- methylpyrazolo-[1,5-a]pyridine-7-carboxamide (Compound 626, 14 mg, 28.5 μmol, 42% yield) as a solid. LCMS (ES, m/z): 492 [M + H]⁺. ¹H NMR (400 MHz, DMSO-d₆) δ 9.81 (s, 1H), 9.37 (s, 1H), 8.77 (dd, J = 3.7, 1.9 Hz, 1H), 7.58 (s, 1H), 7.25 (dd, J = 10.8, 2.0 Hz, 1H), 6.93 (s, 1H), 3.87 (s, 3H), 3.30 (s, 2H), 2.75 (t, J = 11.2 Hz, 2H), 2.55 (s, 3H), 2.27 (s, 3H), 1.96 (s, 1H), 1.86 (s, 1H), 1.77 |
| | | (d, J = 12.6 Hz, 2H), 1.31 (d, J = 11.6 Hz, 2H), 0.21 |
| | | (dt, J = 6.2, 3.0 Hz, 2H), 0.04 (t, J = 3.0 Hz, 2H). |

### Example 193: Synthesis of C223

### Synthesis of C221

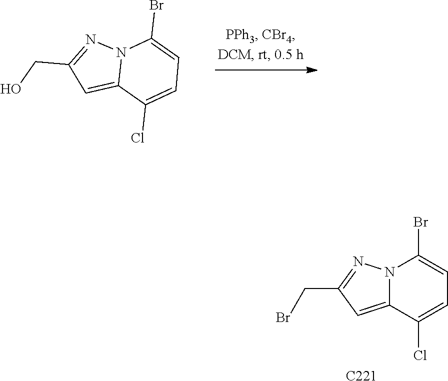 [SMILES: OCc1cc2c(Cl)ccc(Br)n2n1 ; Clc1ccc(Br)n2nc(CBr)cc12 ; "C221"]

To a stirred mixture of (7-bromo-4-chloro-pyrazolo[1,5-a]pyridin-2-yl) methanol (0.14 g, 535.4 μmol) and PPh₃ (182.5 mg, 695.9 μmol) in DCM (2 mL) were added CBr₄ (266.3 mg, 803.1 μmol), and the reaction was stirred for 0.5 h at room temperature under N₂. The resulting mixture was filtered, concentrated under reduced pressure, and purified by silica gel column chromatography (9% EtOAc in PE) to afford 7-bromo-2-(bromomethyl)-4-chloro-pyrazolo[1,5-a]pyridine (C221, 0.12 g, 369.9 μmol, 69% yield) as a solid. LCMS (ES, m/z): 323 [M+H]⁺.

### Synthesis of C222

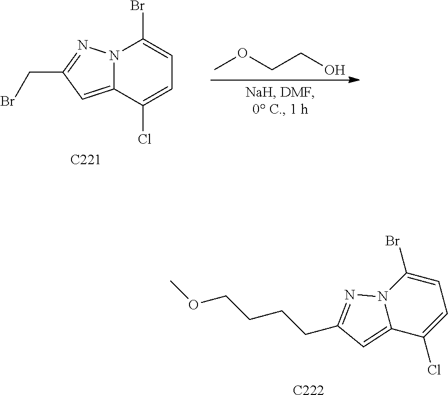

To a solution of 2-methoxyethanol (42.2 mg, 554.9 μmol) in DMF (2 mL) was added NaH (60% dispersion in oil) (16.6 mg, 693.6 μmol). The reaction was additional added 7-bromo-2-(bromomethyl)-4-chloro-pyrazolo[1,5-a]pyridine (0.15 g, 462.4 μmol) and stirred for 1 h at 0° C. The resulting mixture was diluted with NH₄Cl (2 mL) and extracted with DCM (2×8 mL). The combined organic layers were washed with water (2×10 mL), dried over anhydrous Na₂SO₄, and filtered. The filtrate was concentrated under reduced pressure and purified by silica gel column chromatography (17% EtOAc in PE) to afford 7-bromo-4-chloro-2-(2-methoxyethoxymethyl) pyrazolo[1,5-a]pyridine (C222, 0.1 g, 312.9 μmol, 68% yield) as an oil. LCMS (ES, m/z): 317 [M+H]⁺.

### Synthesis of C223

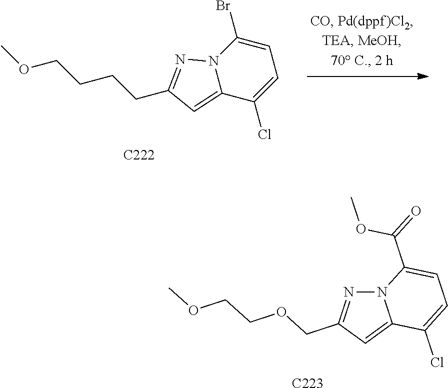 [SMILES: COCCCCc1cc2c(Cl)ccc(Br)n2n1 ; "C222" ; COCCOCc1cc2c(Cl)ccc(C(=O)OC)n2n1 ; "C223"]

To a solution of 7-bromo-4-chloro-2-(2-methoxyethoxymethyl) pyrazolo[1,5-a]pyridine (0.1 g, 312.9 μmol) MeOH (5 mL) was added TEA (158.3 mg, 1.56 mmol) and Pd(dppf)Cl₂ (22.9 mg, 31.29 μmol) in a pressure tank. The mixture was purged with nitrogen for 1 min and then was pressurized to 20 atm with carbon monoxide at 70° C. for 4 h. The mixture was allowed to cool down to room temperature. The insoluble solid was filtered and the filtrate was concentrated under reduced pressure and purified by silica gel column chromatography (50% EtOAc in PE) to afford methyl 4-chloro-2-(2-methoxyethoxymethyl) pyrazolo[1,5-a]pyridine-7-carboxylate (C223, 0.05 g, 167.4 μmol, 53% yield) as a solid. LCMS (ES, m/z): 299 [M+H]⁺.

### Example 194: Synthesis of Compound 467

### Synthesis of Intermediate D2

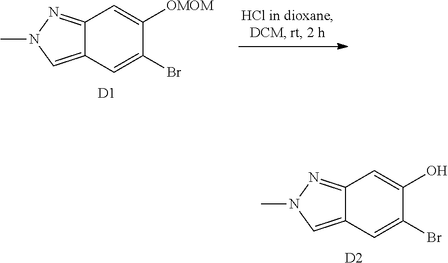

To a solution of 5-bromo-6-(methoxymethoxy)-2-methyl-indazole (D1, 2 g, 7.38 mmol) in DCM (20 mL) was added HCl (4.0 M in 1,4-dioxane) (10 mL). The reaction was stirred for 2 h at room temperature, concentrated under reduced pressure and adjusted to pH 8 with saturated NaHCO₃ (aq.). The resulting mixture was subsequently diluted with water (50 mL) and extracted with DCM/MeOH (10/1) (4×50 mL). The combined organic layers were washed with brine (100 mL), dried over anhydrous Na₂SO₄, and filtered. The filtrate was concentrated under reduced pressure and purified by silica gel column chromatography (9% MeOH in DCM) to afford 5-bromo-2-methyl-indazol-6-ol (D2, 1.5 g, 6.61 mmol). LCMS (ES, m/z): 227 [M+H]⁺.

### Synthesis of Intermediate D3

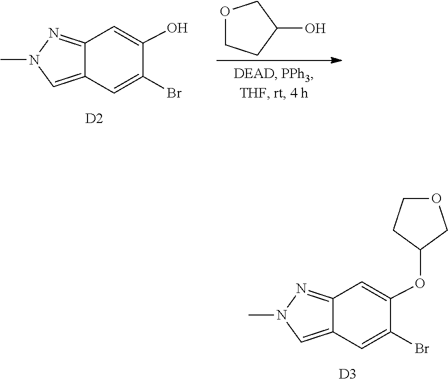

To a stirred mixture of Intermediate D2 (1.1 g, 4.84 mmol) and tetrahydrofuran-3-ol (640.2 mg, 7.27 mmol, 587.4 μL) in THF (15 mL) were added PPh₃ (1.91 g, 7.27 mmol) and DEAD (843.7 mg, 4.84 mmol), and the reaction was stirred for 4 h at room temperature under N₂. The resulting mixture was allowed to cool to room temperature, diluted with water (30 mL), and extracted with EtOAc (3×30 mL). The combined organic layers were washed with water (2×20 mL), brine (20 mL), dried over anhydrous Na₂SO₄, and filtered. The filtrate was concentrated under reduced pressure and purified by silica gel column chromatography (75% EtOAc in PE) to afford 5-bromo-2-methyl-6-tetrahydrofuran-3-yloxy-indazole (D3, 0.65 g, 2.19 mmol). LCMS (ES, m/z): 297 [M+H]⁺.

### Example 195: Synthesis of D8

### Synthesis of Intermediate D4

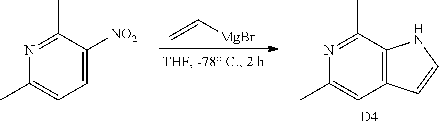

To a stirred mixture of 2,6-dimethyl-3-nitro-pyridine (10 g, 65.7 mmol) in THF (500 mL) was added bromo (vinyl) magnesium (200 mL, 197.2 mmol) dropwise at −78° C. under N₂ and stirred for 2 h. The reaction mixture was quenched by NH₄Cl and extracted with EtOAc (2×500 mL). The combined organic layers were washed with water (3×500 mL), dried over anhydrous Na₂SO₄, and filtered. The filtrate was concentrated under reduced pressure and purified by silica gel column chromatography (17% MeOH in DCM) to afford 5,7-dimethyl-1H-pyrrolo[2,3-c]pyridine (D4, 2.8 g, 19.2 mmol). LCMS (ES, m/z): 147 [M+H]⁺.

### Synthesis of Intermediate D5

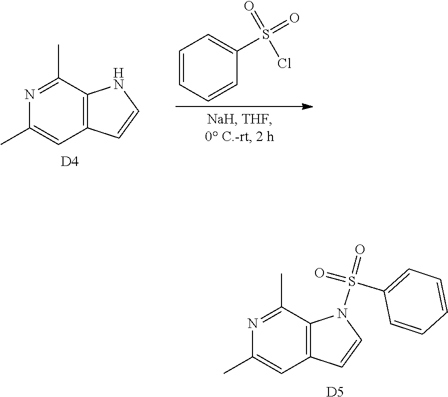

To a solution of Intermediate D4 in THF (60 mL) was added NaH (60% dispersion in oil) (886.5 mg, 36.9 mmol), and the reaction was stirred for 15 min at 0° C. Benzenesulfonyl chloride (6.52 g, 36.9 mmol) was added and the resulting mixture was allowed to warm to room temperature and stirred for 2 h. The reaction mixture was quenched by water (50 mL), extracted with EtOAc (3×50 mL), and the combined organic layers were washed with water (3×150 mL), and dried over anhydrous Na₂SO₄, and filtered. The filtrate was concentrated under reduced pressure and purified by silica gel column chromatography (50% EtOAc in PE) to afford 1-(benzenesulfonyl)-5,7-dimethyl-pyrrolo[2,3-c]pyridine (D5, 2.4 g, 8.38 mmol). LCMS (ES, m/z): 287 [M+H]⁺.

### Synthesis of Intermediate D6

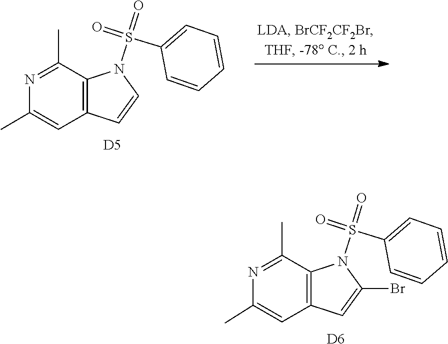

To a solution of Intermediate D5 in THF (10 mL) was added LDA (2.0 M in THF) (1.3 mL, 2.62 mmol) at −78° C. under N₂ atmosphere, and the reaction was stirred for 45 min. 1,2-dibromo-1,1,2,2-tetrafluoroethane (907.4 mg, 3.49 mmol) was added, and the resulting mixture was stirred for additional 2 h at −78° C. under N₂. The reaction mixture was quenched by NH₄Cl, extracted with EtOAc (3×25 mL), and the combined organic layers were washed with water (3×50 mL), dried over anhydrous Na₂SO₄, filtered, and concentrated under reduced pressure to afford 1-(benzenesulfonyl)-2-iodo-5,7-dimethyl-pyrrolo[2,3-c]pyridine (D6, 410 mg, 994.6 μmol). LCMS (ES, m/z): 365 [M+H]⁺.

### Synthesis of Intermediate D7

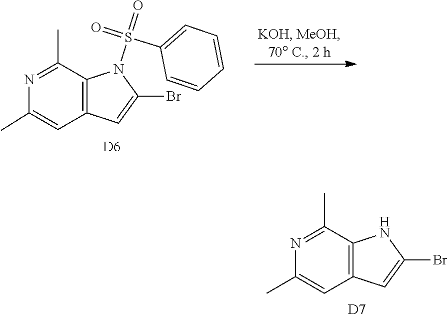

To a stirred mixture of Intermediate D6 (540 mg, 1.48 mmol) in MeOH (25 mL) was added KOH (2.9 g, 51.8 mmol), and the reaction was stirred for 2 h at 70° C. The resulting mixture was diluted with water (30 mL) and extracted with EtOAc (3×30 mL). The combined organic layers were washed with water (3×100 mL) and brine (100 mL), dried over anhydrous Na₂SO₄, and filtered. The filtrate was concentrated under reduced pressure and purified by silica gel column chromatography (9% MeOH in DCM) to afford 2-bromo-5,7-dimethyl-1H-pyrrolo[2,3-c]pyridine (D7, 260 mg, 1.16 mmol. LCMS (ES, m/z): 225 [M+H]⁺.

### Synthesis of Intermediate D8

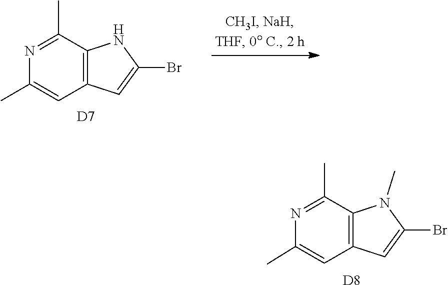

To a solution of Intermediate D7 (260 mg, 1.16 mmol) in DMF (5 mL) was added NaH (60% dispersion in oil) (41.6 mg, 1.73 mmol), and the reaction was stirred for 15 min at 0° C. CH₃I (196.8 mg, 1.39 mmol) was added and the mixture was allowed to warm to room temperature and stirred for 2 h. The reaction mixture was quenched by water, extracted with EtOAc (3×10 mL), and the combined organic layers were washed with water (3×20 mL), dried over anhydrous Na₂SO₄, and filtered. The filtrate was concentrated under reduced pressure and purified by silica gel column chromatography (9% MeOH in DCM) to afford 2-bromo-1,5,7-trimethyl-pyrrolo[2,3-c]pyridine (D8, 160 mg, 669.1 μmol). LCMS (ES, m/z): 239 [M+H]⁺.

### Example 196: Synthesis of D13

### Synthesis of Intermediate D10

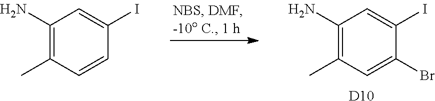

To a stirred mixture of 5-iodo-2-methyl-aniline (9.0 g, 38.6 mmol) in DMF (180 mL) was added NBS (6.8 g, 38.6 mmol) at −10° C. under N₂. The resulting mixture was stirred for 1 h at −10° C. under N₂, then quenched with water (500 mL) at 0° C., and subsequently extracted with EtOAc (2×500 mL). The combined organic layers were washed with water (500 mL), brine (500 mL), dried over anhydrous Na₂SO₄, and filtered. The filtrate was concentrated under reduced pressure and purified by silica gel column chromatography (50% EtOAc in PE) to afford 4-bromo-5-iodo-2-methyl-aniline (D10, 10 g, 32.1 mmol). LCMS (ES, m/z): 310 [M−H]⁻.

### Synthesis of Intermediate D11

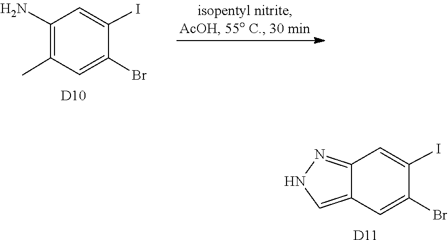

To a stirred mixture of 4-bromo-5-iodo-2-methyl-aniline (10.0 g, 32.1 mmol) in AcOH (50 mL) was added isopentyl nitrite (D10, 4.5 g, 38.47 mmol), and the reaction was stirred for 0.5 h at 55° C. under N₂. The resulting mixture was quenched with water (50 mL) at 25° C., and the precipitated solids were collected by filtration, washed with MeCN (25 mL), and dried to yield 5-bromo-6-iodo-2H-indazole (D11, 9 g, 27.9 mmol). LCMS (ES, m/z): 323 [M+H]⁺.

### Synthesis of Intermediate D12

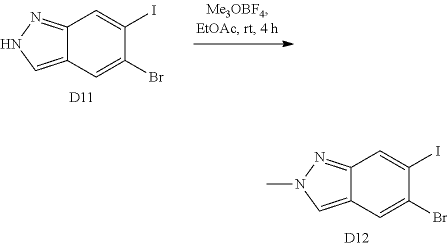

To a solution of Intermediate D11 (9 g, 27.9 mmol) in EtOAc (100 mL) was added Me₃O⁺BF₄⁻ (6.19 g, 41.8 mmol), and the reaction was stirred for 4 h at room temperature. The resulting mixture was filtered, concentrated under reduced pressure, and purified by silica gel column chromatography (50% EtOAc in PE) to afford 5-bromo-6-iodo-2-methyl-indazole (D12, 6 g, 17.8 mmol). LCMS (ES, m/z): 337 [M+H]⁺.

### Synthesis of Intermediate D13

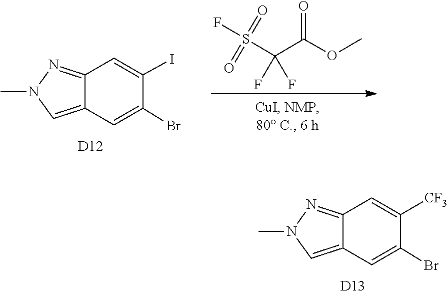

To a stirred mixture of Intermediate D12 (0.5 g, 1.48 mmol) and methyl 2,2-difluoro-2-fluorosulfonyl-acetate (1.71 g, 8.9 mmol) in NMP (5 mL) was added CuI (56.5 mg, 296.8 μmol), and the reaction was stirred for 6 h at 80° C. under N₂. The resulting mixture was allowed to cool to room temperature, diluted with water (10 mL), and extracted with DCM (4×20 mL). The combined organic layers were washed with water (2×40 mL), dried over anhydrous Na₂SO₄, and filtered. The filtrate was concentrated under reduced pressure and purified by silica gel column chromatography (9% EtOAc in PE) to afford 5-bromo-2-methyl-6-(trifluoromethyl)indazole (D13, 0.2 g, 716.7 μmol). LCMS (ES, m/z): 279 [M+H]⁺.

### Example 197: Synthesis of D14

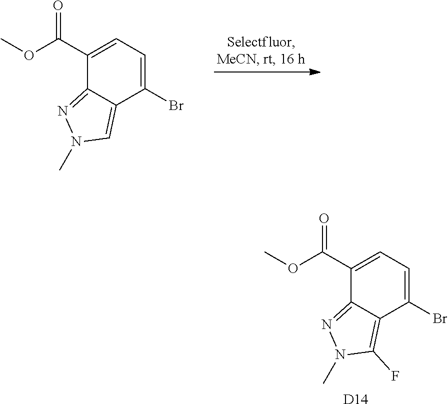

To a stirred mixture of methyl 4-bromo-2-methyl-indazole-7-carboxylate (1.0 g, 3.72 mmol) in MeCN (20 mL) was added Selectfluor (2.6 g, 7.43 mmol) at room temperature. The resulting mixture was stirred for 16 h at 25° C., then quenched with water (100 mL) at room temperature, and subsequently extracted with EtOAc (2×100 mL). The combined organic layers were washed with brine (100 mL), dried over anhydrous Na₂SO₄, and filtered. The filtrate was concentrated under reduced pressure and purified by silica gel column chromatography (50% EtOAc in PE) to afford methyl 4-bromo-3-fluoro-2-methyl-indazole-7-carboxylate (D14, 320 mg, 1.11 mmol). LCMS (ES, m/z): 287 [M+H]⁺.

### Example 198: Synthesis of D16

### Synthesis of Intermediate D15

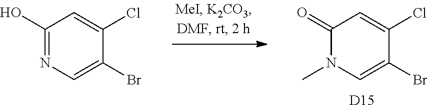

To a stirred mixture of 5-bromo-4-chloro-pyridin-2-ol (100 mg, 479.8 μmol) and K₂CO₃ (79.5 mg, 575.7 μmol) in DMF (2 mL) was added Mel (81.7 mg, 575.7 μmol) at 0° C. The resulting mixture was stirred for 2 h at room temperature, then diluted with water (30 mL), and subsequently extracted with EtOAc (3×30 mL). The combined organic layers were washed with water (2×30 mL) and brine (30 mL), dried over anhydrous Na₂SO₄, and filtered. The filtrate was concentrated under reduced pressure and purified by silica gel column chromatography (50% EtOAc in PE) to afford 5-bromo-4-chloro-1-methyl-pyridin-2-one (D15, 103 mg, 463 μmol). LCMS (ES, m/z): 222 [M+H]⁺.

### Synthesis of Intermediate D16

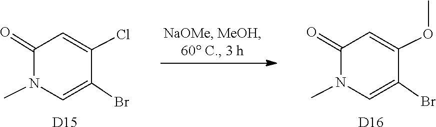

To a stirred solution of Intermediate D15 (95 mg, 427.0 μmol) in MeOH (2 mL) was added NaOMe (92.2 mg, 1.7 mmol) at 0° C. The resulting mixture was stirred for 3 h at 60° C., then diluted with water (30 mL), and extracted with EtOAc (3×20 mL). The combined organic layers were washed with water (2×30 mL) and brine (30 mL), dried over anhydrous Na₂SO₄, and filtered. The filtrate was concentrated under reduced pressure and purified by silica gel column chromatography (33% EtOAc in PE) to afford 5-bromo-4-methoxy-1-methyl-pyridin-2-one (D16, 81 mg, 371.5 μmol). LCMS (ES, m/z): 218 [M+H]⁺.

### Example 199: Synthesis of D18

### Synthesis of Intermediate D17

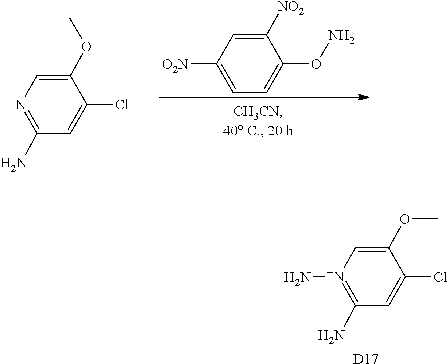

To a stirred mixture of 4-chloro-5-methoxy-pyridin-2-amine (500 mg, 3.15 mmol) and O-(2,4-dinitrophenyl) hydroxylamine (627.8 mg, 3.15 mmol) in MeCN (15 mL), and the reaction was stirred for 20 h at 40° C. The precipitated solids were collected by filtration and washed with MeCN (3×20 mL) to afford 4-chloro-5-methoxy-pyridin-1-ium-1,2-diamine (D17, 900 mg, 3.1 mmol). LCMS (ES, m/z): 174 [M]⁺.

### Synthesis of Intermediate D18

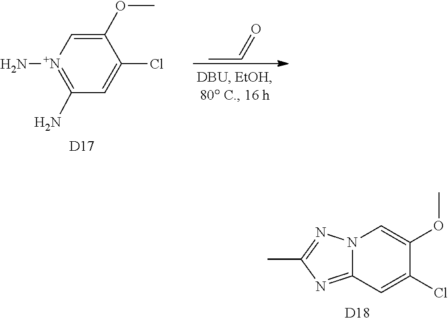

To a stirred mixture of Intermediate D17 (900 mg, 5.15 mmol) in EtOH (20 mL) were added DBU (1.57 g, 10.3 mmol) and acetaldehyde (454.1 mg, 10.3 mmol) dropwise at room temperature. The resulting mixture was stirred for 16 h at 80° C., diluted with water (30 mL), and extracted with EtOAc (3×30 mL). The combined organic layers were washed with water (3×60 mL) and brine (60 mL), dried over anhydrous Na₂SO₄, and filtered. The filtrate was concentrated under reduced pressure and purified by silica gel column chromatography (9% MeOH in DCM) to afford 7-chloro-6-methoxy-2-methyl-[1,2,4]triazolo-[1,5-a]pyridine (D18, 720 mg, 2.19 mmol, 42% yield). LCMS (ES, m/z): 198 [M+H]⁺.

### Example 200: Synthesis of D24

### Synthesis of Intermediate D19

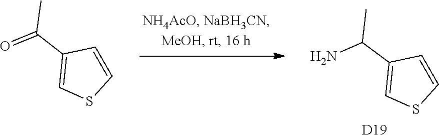

A solution of 1-(3-thienyl) ethanone (23 g, 182.3 mmol) in MeOH (300 mL) was treated with AcONH₄ (140.4 g, 1.8 mol) followed by the addition of NaBH₃CN (34.4 g, 546.9 mmol) at room temperature. The resulting mixture was stirred for 16 h, then diluted with water (100 mL), and subsequently extracted with EtOAc (3×200 mL). The combined organic layers were washed with brine (200 mL), dried over anhydrous Na₂SO₄, and filtered. The filtrate was concentrated under reduced pressure to afford 1-(3-thienyl) ethanamine (D19, 17 g, 106.9 mmol). LCMS (ES, m/z): 128 [M+H]⁺, 169 [M+H+CH₃CN]⁺.

### Synthesis of Intermediate D20

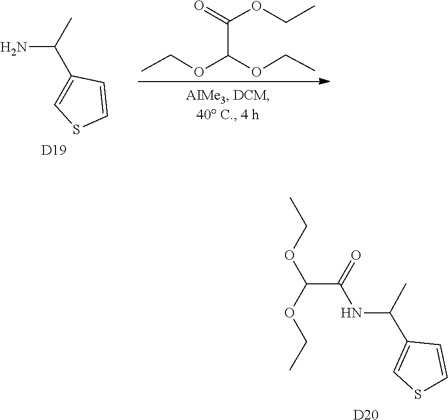

A solution of Intermediate D19 (17 g, 106.9 mmol) in DCM (200 mL) was treated with AlMe₃ (75 mL, 2 M in toluene) for 30 min at 0° C. under N₂ followed by the addition of ethyl 2,2-diethoxyacetate (18.8 g, 106.9 mmol) at 0° C. The resulting mixture was stirred for 4 h at 40° C., then quenched with HCl (1 N) at 0° C., and subsequently extracted with DCM (2×200 mL). The combined organic layers were washed with brine (300 mL), dried over anhydrous Na₂SO₄, and filtered. The filtrate was concentrated under reduced pressure to afford 2,2-diethoxy-N-[1-(3-thienyl)ethyl]acetamide (D20, 20 g, 54.4 mmol). LCMS (ES, m/z): 258 [M+H]⁺.

### Synthesis of Intermediate D21

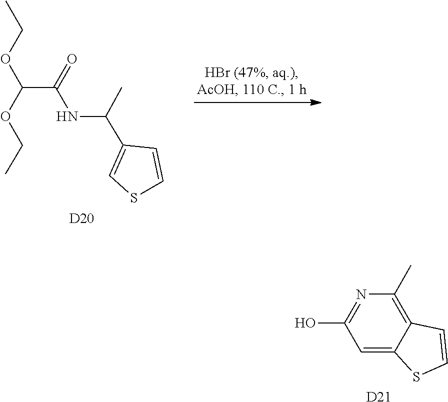

To a stirred mixture of Intermediate D20 (20 g, 54.4 mmol) in AcOH (200 mL) was added HBr (48 wt. % in water) (100 mL) at room temperature. The resulting mixture was stirred for 0.5 h at 110° C., then basified to pH 8 with Na₂CO₃, and subsequently extracted with EtOAc (3×200 mL). The combined organic layers were washed with brine (300 mL), dried over anhydrous Na₂SO₄, and filtered. The filtrate was concentrated under reduced pressure and purified by silica gel column chromatography (50% EtOAc in PE) to afford 4-methylthieno[3,2-c]pyridin-6-ol (D21, 5.2 g, 31.5 mmol). LCMS (ES, m/z): 166 [M+H]⁺.

### Synthesis of Intermediate D22

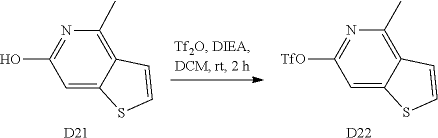 [SMILES: Cc1nc(O)cc2sccc12 ; Cc1nc(OS(=O)(=O)C(F)(F)F)cc2sccc12 ; "D21" ; "D22"]

To a stirred mixture of Intermediate D21 (5 g, 30.3 mmol) and DIEA (23.5 g, 181.6 mmol, 31.6 mL) in DCM (50 mL) was added Tf₂O (25.6 g, 90.8 mmol) at 0° C. The resulting mixture was stirred for 1 h at room temperature, then diluted with water (20 mL), and subsequently extracted with EtOAc (3×30 mL). The combined organic layers were washed with brine (40 mL), dried over anhydrous Na₂SO₄, and filtered. The filtrate was concentrated under reduced pressure and purified by silica gel column chromatography (100% EtOAc) to afford (4-methylthieno[3,2-c]pyridin-6-yl) trifluoromethanesulfonate (D22, 4.8 g, 16.15 mmol). LCMS (ES, m/z): 298 [M+H]⁺.

### Synthesis of Intermediate D23

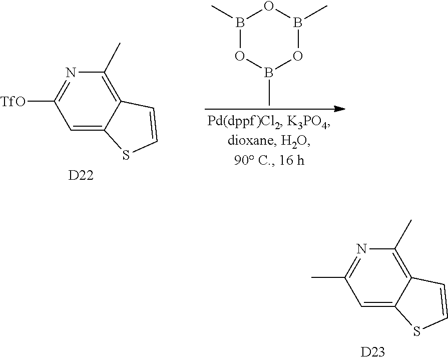

To a stirred mixture of 2,4,6-trimethyl-1,3,5,2,4,6-trioxatriborinane (1.69 g, 13.5 mmol) and Intermediate D22 (4 g, 13.5 mmol) in dioxane (40 mL) and H₂O (4 mL) were added K₃PO₄ (5.71 g, 26.9 mmol) and Pd(dppf)Cl₂—CH₂Cl₂ (1.1 g, 1.35 mmol), and the reaction was stirred for 16 h at 90° C. under N₂. The mixture was purified by silica gel column chromatography (20% EtOAc in PE) to afford 4,6-dimethylthieno[3,2-c]pyridine (D23, 580 mg, 3.55 mmol). LCMS (ES, m/z): 164 [M+H]⁺.

### Synthesis of Intermediate D24

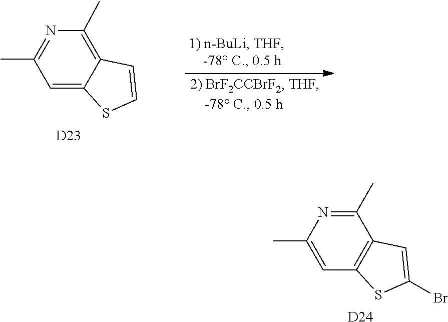

A solution of Intermediate D23 (200 mg, 1.23 mmol) in THF (4 mL) was treated with n-BuLi (2.5 M in hexanes) (0.79 mL, 2.5 mmol) for 30 min at −78° C. under N₂ followed by the addition of BrF₂CCBrF₂ (694.9 mg, 3.1 mmol) at −78° C. The resulting mixture was stirred for 1 h at −78° C., then quenched with sat. NH₄Cl (aq.) at 0° C., and subsequently extracted with EtOAc (2×10 mL). The combined organic layers were washed with brine (20 mL), dried over anhydrous Na₂SO₄, and filtered. The filtrate was concentrated under reduced pressure and purified by silica gel column chromatography (25% EtOAc in PE) to afford 2-bromo-4,6-dimethyl-thieno[3,2-c]pyridine (D24, 150 mg, 619.5 μmol). LCMS (ES, m/z): 242 [M+H]⁺.

### Example 201: Synthesis of D30

### Synthesis of Intermediate D25

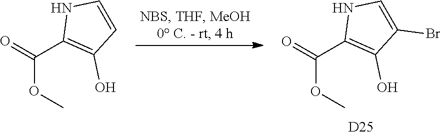

To a solution of methyl 3-hydroxy-1H-pyrrole-2-carboxylate (19 g, 134.6 mmol) in THF (190 mL) and MeOH (190 mL) was added NBS (35.9 g, 202 mmol), and the reaction was stirred for 4 h at 0° C. The resulting mixture was diluted with H₂O (800 mL) and extracted with EtOAc (2×800 mL). The combined organic layers were washed with water (2×600 mL), dried over anhydrous Na₂SO₄, and filtered. The filtrate was concentrated under reduced pressure and purified by silica gel column chromatography (20% EtOAc in PE) to afford methyl 4-bromo-3-hydroxy-1H-pyrrole-2-carboxylate (D25, 17.9 g, 81.4 mmol). LCMS (ES, m/z): 220 [M+H]⁺.

### Synthesis of Intermediate D26

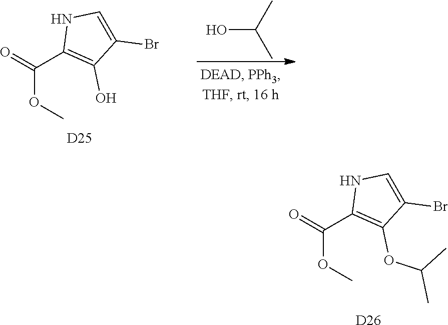

To a solution of Intermediate D25 (10 g, 45.5 mmol) in THF (100 mL) were added propan-2-ol (2.73 g, 45.5 mmol) and PPh₃ (17.9 g, 68.2 mmol) at room temperature. Then DEAD (11.9 g, 68.2 mmol) was added dropwise at 0° C. under N₂. The mixture was stirred for 16 h at room temperature, diluted with H₂O (200 mL), and extracted with EtOAc (2×200 mL). The combined organic layers were washed with H₂O (2×200 mL), dried over anhydrous Na₂SO₄, and filtered. The filtrate was concentrated under reduced pressure and purified by silica gel column chromatography (25% EtOAc in PE) to afford methyl 4-bromo-3-isopropoxy-1H-pyrrole-2-carboxylate (D26, 10.1 g, 38.5 mmol). LCMS (ES, m/z): 262 [M+H]⁺.

### Synthesis of Intermediate D27

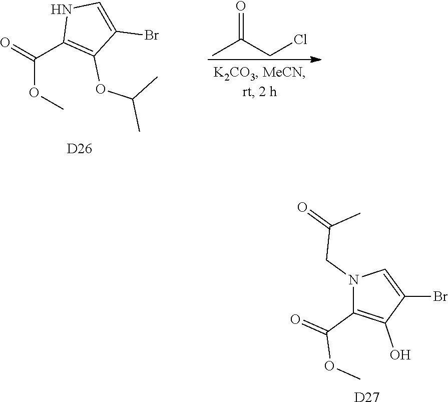

To a solution of Intermediate D26 (10 g, 38.2 mmol) in MeCN (100 mL) were added 1-chloropropan-2-one (4.24 g, 45.8 mmol) and K₂CO₃ (10.6 g, 76.3 mmol) at room temperature. The mixture was stirred for 2 h, then diluted with H₂O (200 mL), and subsequently extracted with EtOAc (2×150 mL). The combined organic layers were washed with H₂O (2×200 mL), dried over anhydrous Na₂SO₄, and filtered. The filtrate was concentrated under reduced pressure and purified by silica gel column chromatography (17% EtOAc in PE) to afford methyl 1-acetonyl-4-bromo-3-isopropoxy-pyrrole-2-carboxylate (D27, 9.2 g, 28.9 mmol). LCMS (ES, m/z): 318 [M+H]⁺.

### Synthesis of Intermediate D28

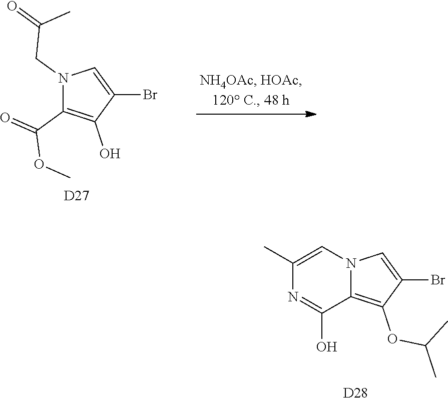

To a solution of Intermediate D27 (4.4 g, 13.8 mmol) in HOAc (50 mL) was added NH₄OAc (15.99 g, 207.4 mmol, 13.7 mL), and the reaction was stirred for 48 h at 120° C. The resulting mixture was concentrated under reduced pressure. The reaction was diluted with EtOAc (50 mL), washed with NaHCO₃ (aq.) (3×50 mL), dried over anhydrous Na₂SO₄, and filtered. The filtrate was concentrated under reduced pressure and purified by silica gel column chromatography (25% EtOAc in PE) to afford 7-bromo-8-isopropoxy-3-methyl-pyrrolo[1,2-a]pyrazin-1-ol (D28, 1.2 g, 4.2 mmol). LCMS (ES, m/z): 285 [M+H]⁺.

### Synthesis of Intermediate D29

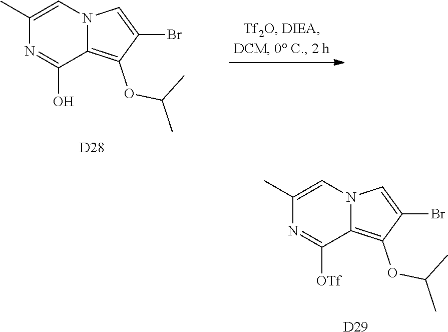 [SMILES: Cc1cn2cc(Br)c(OC(C)C)c2c(O)n1 ; "D28" ; Cc1cn2cc(Br)c(OC(C)C)c2c(OS(=O)(=O)C(F)(F)F)n1 ; "D29"]

To a solution of Intermediate D28 (1.1 g, 3.9 mmol) in DCM (11 mL) was added DIEA (1.99 g, 15.4 mmol) at room temperature. Then Tf₂O (2.18 g, 7.7 mmol, 1.3 mL) was added at 0° C. The mixture was stirred for 2 h at 0° C., then diluted with H₂O (20 mL), and subsequently extracted with DCM (2×20 mL). The combined organic layers were washed with H₂O (2×20 mL), dried over anhydrous Na₂SO₄, and filtered. The filtrate was concentrated under reduced pressure and purified by silica gel column chromatography (14% EtOAc in PE) to afford (7-bromo-8-isopropoxy-3-methyl-pyrrolo[1,2-a]pyrazin-1-yl) trifluoromethanesulfonate (D29, 1 g, 2.4 mmol). LCMS (ES, m/z): 417 [M+H]⁺.

### Synthesis of Intermediate D30

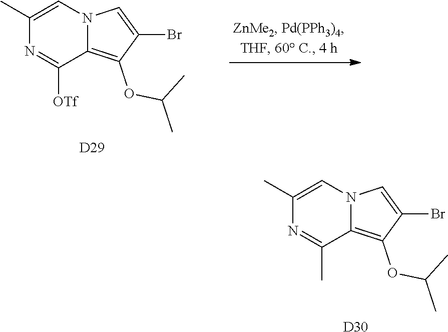 [SMILES: Cc1cn2cc(Br)c(OC(C)C)c2c(OS(=O)(=O)C(F)(F)F)n1 ; "D29" ; Cc1cn2cc(Br)c(OC(C)C)c2c(C)n1 ; "D30"]

To a stirred mixture of Intermediate D29 (270 mg, 647.2 μmol) in THF (3 mL) was added Pd(PPh₃)₄ (74.8 mg, 64.7 μmol) under N₂. Then dimethylzinc (1 M, 800 μL) was added at room temperature still under N₂. The mixture was stirred for 4 h at 60° C., then diluted with H₂O (10 mL), and later extracted with EtOAc (2×10 mL). The combined organic layers were washed with H₂O (2×10 mL), dried over anhydrous Na₂SO₄, and filtered. The filtrate was concentrated under reduced pressure and purified by silica gel column chromatography (20% EtOAc in PE) to afford 7-bromo-8-isopropoxy-1,3-dimethyl-pyrrolo[1,2-a]pyrazine (D30, 70 mg, 247.2 μmol). LCMS (ES, m/z): 283 [M+H]⁺.

### Example 202: Synthesis of D33

### Synthesis of Intermediate D31

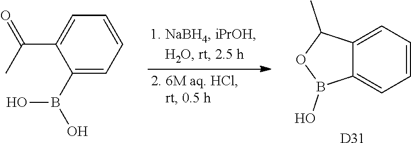

To a solution of (2-acetylphenyl) boronic acid (900 mg, 5.49 mmol, 1 eq) in i-PrOH: H₂O (3:1, 40 mL), NaBH₄ (830.6 mg, 21.96 mmol, 4 eq) was added in portions. The reaction solution was allowed to stir for 2.5 h at room temperature then the reaction was quenched by dropwise addition of 1M HCl until gas evolution ceased and the reaction mixture reached ~pH 1-2. Once the pH was established, the aqueous solution was allowed to stir for an additional 0.5 h then extracted with DCM. The combined organic layers were dried over anhydrous Na₂SO₄, and filtered. The filtrate was concentrated and purified by silica gel column chromatography (30% EtOAc in PE) to afford 1-hydroxy-3-methyl-3H-2,1-benzoxaborole (D31, 720 mg, 4.87 mmol). LCMS (ES, m/z): 165.2 [M−H+H₂O]⁻.

### Synthesis of Intermediate D32

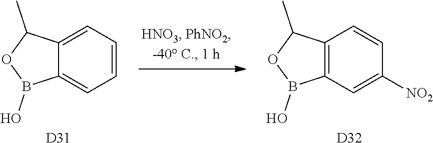

To HNO₃ fuming (23 g, 365 mmol) at −45° C. was added a solution of Intermediate D31 (720 mg, 4.87 mmol, 1 eq) in PhNO₂ (9 mL) slowly via a syringe while maintaining the reaction temperature between −40 to −45° C. Once the addition was complete the resulting solution was allowed to stir at −45° C. for an additional 1 h before pouring into crushed ice (50 g). The ice mixture was allowed to melt and the aqueous solution was extracted with DCM. The combined organic layers were dried over Na₂SO₄, filtered, and concentrated. The crude oil was diluted with DCM: PE (1 L, 1:1). The solution was concentrated under reduced pressure to half the volume, and the resulting solution was allowed to stand at −20° C. The precipitate formed was filtered out, washed with PE, and vacuum-dried to give 1-hydroxy-3-methyl-6-nitro-3H-2,1-benzoxaborole (D32, 370 mg, 1.92 mmol). LCMS (ES, m/z): 210.1 [M−H+H₂O]—.

### Synthesis of Intermediate D33

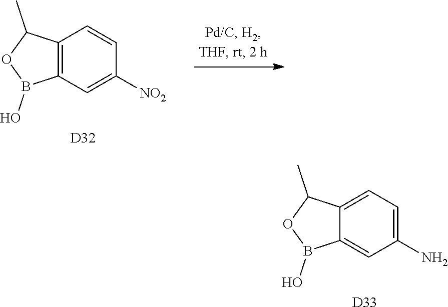

To a solution of Intermediate D32 (370 mg, 1.92 mmol, 1 eq) in MeOH (4 mL) was added HCl (4.0 M in EtOAc, 0.48 mL) and Pd/C (41 mg) under N₂. The mixture was hydrogenated at room temperature for 16 h under hydrogen atmosphere, filtered, and the solid was washed with MeOH (4 mL). The filtrate was concentrated under reduced pressure to afford 1-hydroxy-3-methyl-3H-2,1-benzoxaborol-6-amine (D33, 170 mg, 1.04 mmol). LCMS (ES, m/z): 164.1 [M+H]⁺.

### Example 203: Synthesis of D38

### Synthesis of Intermediate D34

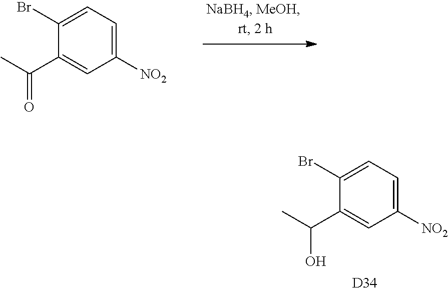

To a stirred solution of 1-(2-bromo-5-nitro-phenyl) ethanone (4 g, 16.4 mmol) in MeOH (100 mL) was added NaBH₄ (930 mg, 24.6 mmol) in portions at 0° C. The resulting mixture was stirred for 2 h at room temperature, then diluted with water (100 mL), and subsequently extracted with EtOAc (2×50 mL). The organic layer was combined, washed with brine (50 mL), dried over Na₂SO₄, and filtered. The filtrate was concentrated under reduced pressure and purified by silica gel column chromatography (10%-25% EtOAc in PE) to afford 1-(2-bromo-5-nitro-phenyl)ethanol (D34, 3.1 g, 12.6 mmol). ¹H NMR (400 MHz, DMSO-d₆) δ 8.37 (s, 1H), 8.05-8.02 (m, 1H), 7.89 (d, J=8.0 Hz, 1H), 5.80 (s, 1H), 5.03-4.97 (m, 1H), 1.35 (d, J=8.0 Hz, 3H).

### Synthesis of Intermediate D35

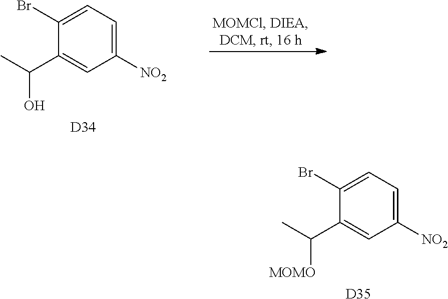

To a stirred solution of Intermediate D34 (3.1 g, 12.6 mmol) and DIEA (3.3 g, 25.2 mmol, 4.4 mL) in DCM (60 mL) was added chloro(methoxy) methane (2.03 g, 25.2 mmol) dropwise, and the reaction was stirred for 16 h at room temperature. The resulting mixture was washed with sat. NaHCO₃ (10 mL), brine (10 mL), dried over Na₂SO₄, and filtered. The filtrate was concentrated under reduced pressure and purified by silica gel column chromatography to afford 1-bromo-2-[1-(methoxymethoxy)-ethyl]-4-nitro-benzene (D35, 3.3 g, 11.4 mmol). ¹H NMR (400 MHz, CDCl₃) δ 8.44-8.41 (m, 1H), 8.01-7.89 (m, 1H), 7.73-7.71 (m, 1H), 5.20-5.14 (m, 1H), 4.69-4.66 (m, 1H), 4.60-4.58 (m, 1H), 3.40 (s, 3H), 1.49 (d, J=3.2 Hz, 3H).

### Synthesis of Intermediate D36

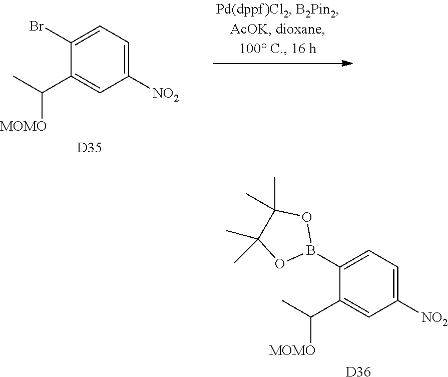

To a stirred solution of Intermediate D35 (3.3 g, 11.4 mmol) and 4,4,5,5-tetramethyl-2-(4,4,5,5-tetramethyl-1,3,2-dioxaborolan-2-yl)-1,3,2-dioxaborolane (3.47 g, 13.7 mmol) in dioxane (70 mL) were added Pd(dppf)Cl₂ (832 mg, 1.14 mmol) and AcOK (2.23 g, 22.8 mmol, 1.4 mL). The resulting mixture was degassed with N₂, and the reaction was stirred for 16 h at 100° C. The formed precipitate was removed by filtration, and the filtrate was concentrated under reduced pressure and purified by silica gel column chromatography (5%-20% EtOAc in PE) to afford 2-[2-[1-(methoxymethoxy)ethyl]-4-nitro-phenyl]-4,4,5,5-tetramethyl-1,3,2-dioxaborolane (D36, 2.6 g, 7.7 mmol). ¹H NMR (400 MHz, CDCl₃) δ 8.38 (s, 1H), 8.08-8.05 (m, 1H), 7.89 (d, J=8.0 Hz, 1H), 5.48-5.41 (m, 1H), 4.69-4.57 (m, 2H), 3.37 (s, 3H), 1.39 (s, 3H), 1.28 (s, 12H).

### Synthesis of Intermediate D37

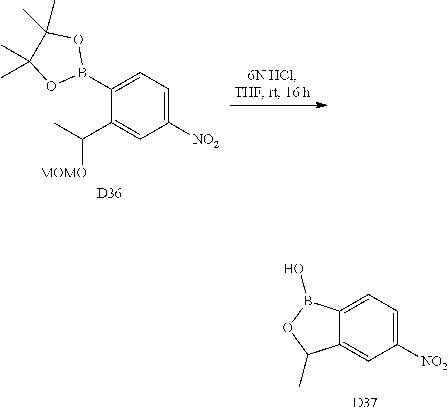

To a stirred solution of Intermediate D36 (2.6 g, 7.7 mmol) in THF (10 mL) was added 6N HCl (5 mL). The resulting mixture was stirred at room temperature for 16 h, then diluted with water (30 mL), and the aqueous phase was extracted with EtOAc (3×20 mL). The organic layer was combined, washed with water (20 mL), brine (20 mL), dried over Na₂SO₄ and filtered. The filtrate was concentrated under reduced pressure and purified by silica gel column chromatography (10-80% EtOAc in PE) to afford 1-hydroxy-3-methyl-5-nitro-3H-2,1-benzoxaborole (D37, 1 g, 5.2 mmol). ¹H NMR (400 MHz, DMSO-d₆) δ 9.56 (s, 1H), 8.31-8.29 (m, 1H), 8.22-8.19 (m, 1H), 7.95 (d, J=8.0 Hz, 1H), 5.38-5.33 (m, 1H), 1.48 (d, J=6.8 Hz, 3H).

### Synthesis of Intermediate D37

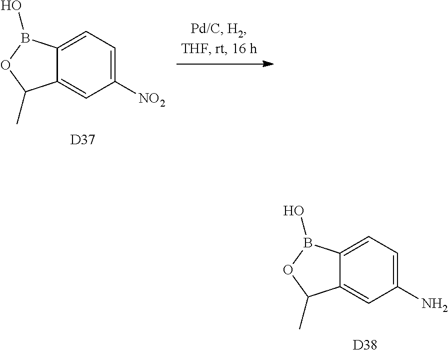

To a stirred solution of Intermediate D37 (1 g, 5.2 mmol) in THF (20 mL) was added Pd/C (100 mg, 939.7 μmol). The reaction was degassed and flushed with H₂ (×3), then stirred at room temperature for 16 h under balloon pressure. The reaction mixture was passed through a pad of celite, the filtrate was concentrated and purified by silica gel column chromatography (CH₃CN in water (0.05% TFA), 10%-40% in 10 min) to afford 1-hydroxy-3-methyl-3H-2,1-benzoxaborol-5-amine (D38, 400 mg, 2.5 mmol). ¹H NMR (400 MHz, DMSO-d₆) δ 8.53 (s, 1H), 7.31 (d, J=8.0 Hz, 1H), 6.51-6.47 (m, 1H), 6.44 (s, 1H), 5.43 (s, 2H), 5.00-4.93 (m, 1H), 1.31 (d, J=8.0 Hz, 3H).

### Example 204: Synthesis of D43

### Synthesis of Intermediate D39

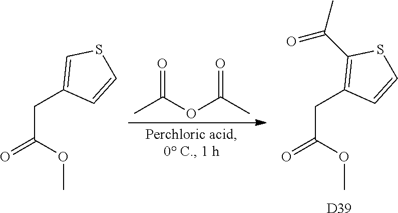

To a stirred mixture of acetyl acetate (3.27 g, 32.0 mmol, 3.02 mL) in perchloric acid (322 mg, 3.20 mmol) was added methyl 2-(3-thienyl)acetate (5 g, 32.01 mmol) at 0° C. The resulting mixture was stirred for 1 h at 0° C., then diluted with water (50 mL), neutralized to pH 6 with NaHCO₃ (aq.), and later extracted with EtOAc (2×50 mL). The resulting mixture was concentrated under reduced pressure to afford methyl 2-(2-acetyl-3-thienyl)acetate (D39, 5.5 g, 27.7 mmol). LCMS (ES, m/z): 199 [M+H]⁺.

### Synthesis of Intermediate D40

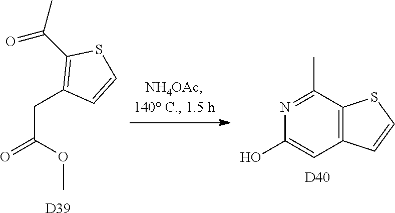

Intermediate D39 (5.5 g, 27.7 mmol) was added to molten NH₄OAc (53.5 g, 693.6 mmol), and the reaction was stirred for 1.5 h at 140° C. The resulting mixture was then poured into ice water and the precipitate was collected by filtration and washed with water. The solid was purified by trituration with PE/EtOAc (5/1) to afford 7-methylthieno[2,3-c]pyridin-5-ol (D40, 2 g, 12.1 mmol). LCMS (ES, m/z): 166 [M+H]⁺.

### Synthesis of Intermediate D41

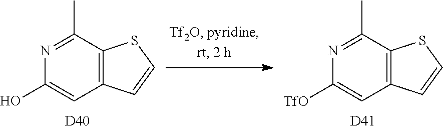

To a solution of Intermediate D40 (1.8 g, 10.9 mmol) in pyridine (18 mL) was added Tf₂O (9.22 g, 32.7 mmol, 5.5 mL) at 0° C. The mixture was stirred for 2 h at room temperature, then diluted with H₂O (30 mL), and later extracted with DCM (2×30 mL). The combined organic layers were washed with H₂O (2×40 mL), dried over anhydrous Na₂SO₄, and filtered. The filtrate was concentrated under reduced pressure and purified by silica gel column chromatography (1% EtOAc in PE) to afford (7-methylthieno[2,3-c]pyridin-5-yl) trifluoromethanesulfonate (D41, 2.5 g, 8.4 mmol). LCMS (ES, m/z): 298 [M+H]⁺.

### Synthesis of Intermediate D42

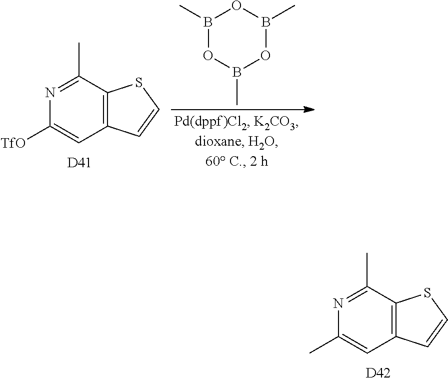

To a stirred mixture of Intermediate D41 (2.4 g, 8.1 mmol) in dioxane (25 mL)/H₂O (2.5 mL) were added 2,4,6-trimethyl-1,3,5,2,4,6-trioxatriborinane (3.04 g, 24.2 mmol), K₂CO₃ (4.5 g, 32.3 mmol) and Pd(dppf)Cl₂ (659.3 mg, 807.3 μmol) at room temperature under N₂. The mixture was stirred for 2 h at 60° C., then diluted with H₂O (30 mL), and later extracted with EtOAc (2×30 mL). The combined organic layers were washed with H₂O (2×40 mL), dried over anhydrous Na₂SO₄, and filtered. The filtrate was concentrated under reduced pressure and purified by silica gel column chromatography (17% EtOAc in PE) to afford 5,7-dimethylthieno[2,3-c]pyridine (D42, 600 mg, 3.7 mmol). ¹H NMR (300 MHz, DMSO-d₆) δ 8.01 (d, J=5.4 Hz, 1H), 7.52 (s, 1H), 7.45 (d, J=5.4 Hz, 1H), 2.66 (s, 3H), 2.53 (s, 3H).

### Synthesis of Intermediate D43

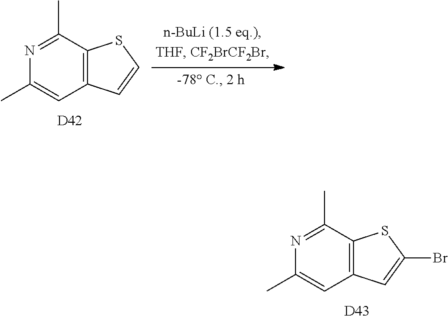

To a solution of Intermediate D42 (400 mg, 2.45 mmol) in THF (8 mL) was added n-BuLi (2.5 M in hexanes) (2.5 M, 1.47 mL) dropwise at −78° C. under N₂. The mixture was stirred for 1 h at −78° C. under N₂ before CF₂Br₂CF₂Br (1.27 g, 4.9 mmol) was added. The mixture was then stirred for 1 h at −78° C. under N₂, then quenched with NH₄Cl (aq.) at 0° C., and later extracted with DCM (2×30 mL). The combined organic layers were washed with H₂O (2×30 mL), dried over anhydrous Na₂SO₄, and filtered. The filtrate was concentrated under reduced pressure and purified by silica gel column chromatography (11% EtOAc in PE) to afford 2-bromo-5,7-dimethyl-thieno[2,3-c]pyridine (D43, 440 mg, 1.82 mmol). LCMS (ES, m/z): 242 [M+H]⁺.

### Example 205: Synthesis of D46

### Synthesis of Intermediate D44

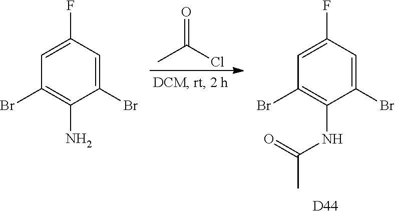

To a solution of 2,6-dibromo-4-fluoro-aniline (30 g, 111.6 mmol) in DCM (300 mL) was added acetyl chloride (10.5 g, 133.9 mmol). The reaction was stirred for 2 h at room temperature, then diluted with water (300 mL) and extracted with DCM (3×200 mL). The combined organic layers were washed with water (500 mL), brine (500 mL) and dried over anhydrous Na₂SO₄, and filtered. The filtrate was concentrated under reduced pressure and purified by silica gel column chromatography (50% EtOAc in PE) to afford N-(2,6-dibromo-4-fluoro-phenyl) acetamide (D44, 25 g, 80.4 mmol). LCMS (ES, m/z): 310 [M+H]⁺.

### Synthesis of Intermediate D45

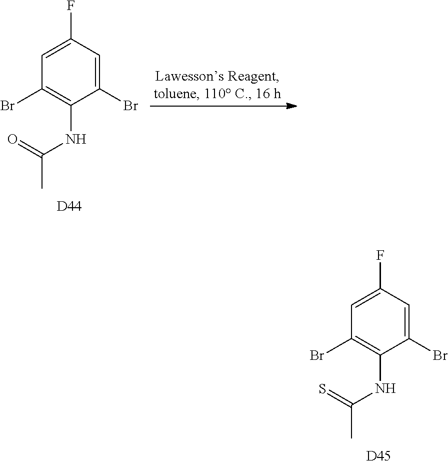

To a solution of Intermediate D44 (23 g, 74 mmol) in toluene (300 mL) was added Lawessons reagent (44.9 g, 111 mmol). The reaction was stirred for 16 h at 110° C., was allowed to cool to room temperature, then diluted with water (200 mL), and subsequently extracted with EtOAc (2×200 mL). The combined organic layers were washed with water (2×400 mL), dried over anhydrous Na₂SO₄, and filtered. The filtrate was concentrated under reduced pressure and purified by silica gel column chromatography (50% EtOAc in PE) to afford N-(2,6-dibromo-4-fluoro-phenyl)thioacetamide (D45, 18 g, 55.0 mmol). LCMS (ES, m/z): 326 [M+H]⁺.

### Synthesis of Intermediate D46

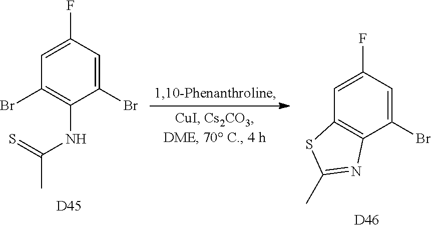

To a stirred mixture of Intermediate D45 (18 g, 55.0 mmol) in DME (200 mL) were added Cs₂CO₃ (35.9 g, 110.1 mmol), 1,10-phenanthroline (1.98 g, 11.0 mmol) and CuI (1.05 g, 5.5 mmol) at room temperature under N₂. The resulting mixture was stirred for 4 h at 70° C., allowed to cool to room temperature, diluted with water (100 mL), and then extracted with EtOAc (2×200 mL). The combined organic layers were washed with water (2×400 mL), dried over anhydrous Na₂SO₄, and filtered. The filtrate was concentrated under reduced pressure and purified by silica gel column chromatography (50% EtOAc in PE) to afford 4-bromo-6-fluoro-2-methyl-1,3-benzothiazole (D46, 11 g, 44.7 mmol). LCMS (ES, m/z): 246 [M+H]⁺.

### Example 206: Synthesis of D52

### Synthesis of Intermediate D51

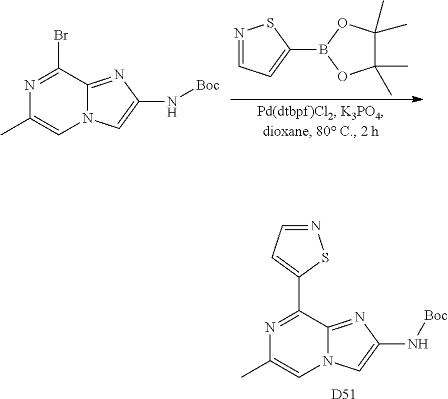

To a solution of tert-butyl N-(8-bromo-6-methyl-imidazo[1,2-a]pyrazin-2-yl)carbamate (200 mg, 611.3 μmol) and 5-(4,4,5,5-tetramethyl-1,3,2-dioxaborolan-2-yl) isothiazole (167.7 mg, 794.7 μmol) in dioxane (4 mL) and H₂O (0.4 mL) were added K₃PO₄ (129.7 mg, 611.3 μmol) and Pd(dtbpf)Cl₂ (198.6 mg, 305.7 μmol), and the reaction was stirred for 2 h at 80° C. under N₂. The resulting mixture was concentrated under reduced pressure and purified by silica gel column chromatography (50% EtOAc in PE) to afford tert-butyl N-(8-isothiazol-5-yl-6-methylimidazo[1,2-a]pyrazin-2-yl)carbamate (D51, 60 mg, 181.1 μmol). LCMS (ES, m/z): 332 [M+H]⁺.

### Synthesis of Intermediate D52

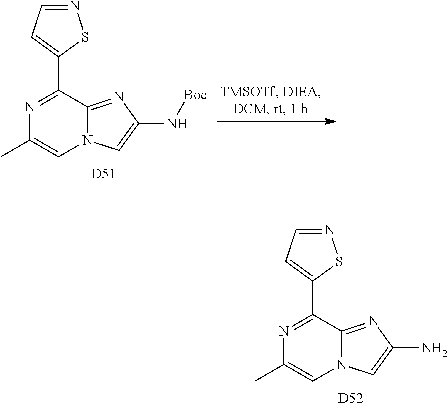

To a solution of Intermediate D51 (55.0 mg, 166 μmol) in DCM (1 mL) was added TMSOTf (110.6 mg, 497.9 μmol) and DIEA (42.9 mg, 331.9 μmol). The reaction was stirred for 1 h at room temperature, then concentrated under reduced pressure, and adjusted to pH 8 with saturated NaHCO₃ (aq.). The resulting mixture was diluted with water (5 mL) and extracted with DCM/MeOH (20/1) (2×10 mL). The combined organic layers were washed with water (20 mL), brine (20 mL), dried over anhydrous Na₂SO₄, filtered, and concentrated under reduced pressure to afford 8-isothiazol-5-yl-6-methyl-imidazo[1,2-a]pyrazin-2-amine (D52, 36 mg, 155.7 μmol). LCMS (ES, m/z): 232 [M+H]⁺.

An analogous method was followed to obtain the following compounds.

| | Starting | |
|---|---|---|
| Compound | Material | Characterization |
| 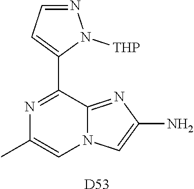 | 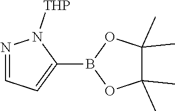 | Modification: Step 1 was ran for 1 h. The filtrate was concentrated under reduced pressure, resulting in 6-methyl-8-(2-tetrahydropyran-2-yl-pyrazol-3-yl)imidazo [1,2-a]-pyrazin-2-amine (D53, 130 mg, 435.7 μmol). LCMS (ES, m/z): 299 [M + H]⁺. |
| 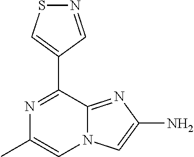 [SMILES: Cc1cn2cc(N)nc2c(-c2cnsc2)n1] | 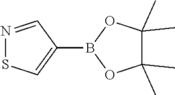 | The filtrate was concentrated under reduced pressure, resulting in 8-isothiazol-4- yl-6-methyl-imidazo[1,2-a]-pyrazin-2-amine (D54, 130 mg, 562.1 μmol). LCMS (ES, m/z): 232 [M + H]⁺. |
| D54 | | |

### Example 207: Synthesis of Compound 679

### Synthesis of Intermediate D55

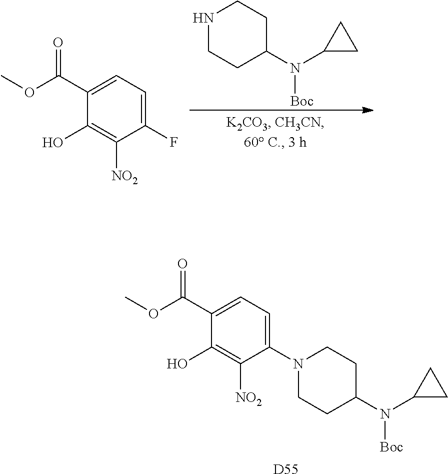 [SMILES: COC(=O)c1ccc(F)c([N+](=O)[O-])c1O ; CC(C)(C)OC(=O)N(C1CCNCC1)C1CC1 ; COC(=O)c1ccc(N2CCC(N(C(=O)OC(C)(C)C)C3CC3)CC2)c([N+](=O)[O-])c1O ; "D55"]

To a stirred mixture of methyl 4-fluoro-2-hydroxy-3-nitro-benzoate (2 g, 9.3 mmol) and tert-butyl N-cyclopropyl-N-(4-piperidyl)carbamate (2.23 g, 9.3 mmol) in MeCN (40 mL) was added K₂CO₃ (3.85 g, 27.9 mmol), and the reaction was stirred for 3 h at 60° C. under N₂. The resulting mixture was concentrated under reduced pressure and purified by silica gel column chromatography (67% EtOAc in PE) to afford methyl 4-[4-[tert-butoxycarbonyl(cyclopropyl)-amino]-1-piperidyl]-2-hydroxy-3-nitro-benzoate (D55, 2.4 g, 5.5 mmol). LCMS (ES, m/z): 436 [M+H]⁺.

### Synthesis of Intermediate D56

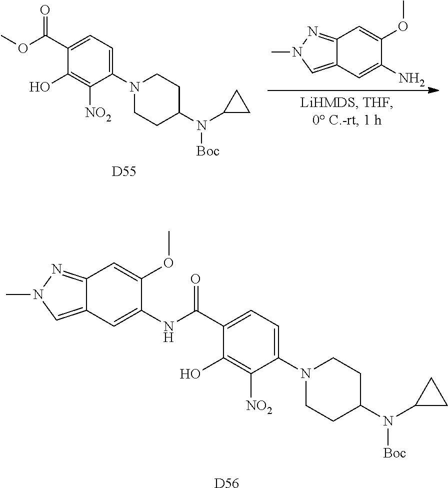 [SMILES: COC(=O)c1ccc(N2CCC(N(C(=O)OC(C)(C)C)C3CC3)CC2)c([N+](=O)[O-])c1O ; COc1cc2nn(C)cc2cc1N ; COc1cc2nn(C)cc2cc1NC(=O)c1ccc(N2CCC(N(C(=O)OC(C)(C)C)C3CC3)CC2)c([N+](=O)[O-])c1O ; "D55" ; "D56"]

To a stirred mixture of Intermediate D55 (800 mg, 1.84 mmol) and 6-methoxy-2-methyl-indazol-5-amine (325.5 mg, 1.84 mmol) in THF (16 mL) was added LiHMDS (9 mL, 9.19 mmol) at 0° C. under N₂. The resulting mixture was stirred for 1 h at room temperature, then diluted with cold water (10 mL), neutralized to pH 5 with HCl (aq.), and the aqueous layer was extracted with EtOAc (2×10 mL). The resulting mixture was concentrated under reduced pressure to afford tert-butyl N-cyclopropyl-N-[1-[3-hydroxy-4-[(6-methoxy-2-methyl-indazol-5-yl)carbamoyl]-2-nitro-phenyl]-4-piperidyl]carbamate (D56, 500 mg, 861.1 μmol). LCMS (ES, m/z): 581 [M+H]⁺.

### Synthesis of Intermediate D57

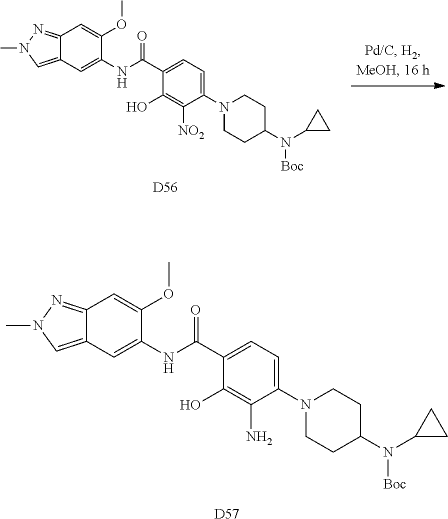 [SMILES: COc1cc2nn(C)cc2cc1NC(=O)c1ccc(N2CCC(N(C(=O)OC(C)(C)C)C3CC3)CC2)c([N+](=O)[O-])c1O ; COc1cc2nn(C)cc2cc1NC(=O)c1ccc(N2CCC(N(C(=O)OC(C)(C)C)C3CC3)CC2)c(N)c1O ; "D56" ; "D57"]

A stirred mixture of Intermediate D56 (500 mg, 861.1 μmol) in MeOH (10 mL) was treated with Pd/C (183.2 mg) at room temperature. The resulting mixture was stirred for 2 h at room temperature under hydrogen atmosphere, the Pd/C was eliminated by filtration, and the mixture was concentrated under reduced pressure to afford tert-butyl N-[1-[2-amino-3-hydroxy-4-[(6-methoxy-2-methyl-indazol-5-yl)carbamoyl]phenyl]-4-piperidyl]-N-cyclopropyl-carbamate (D57, 400 mg, 726.4 μmol). LCMS (ES, m/z): 551 [M+H]⁺.

### Synthesis of Intermediate D58

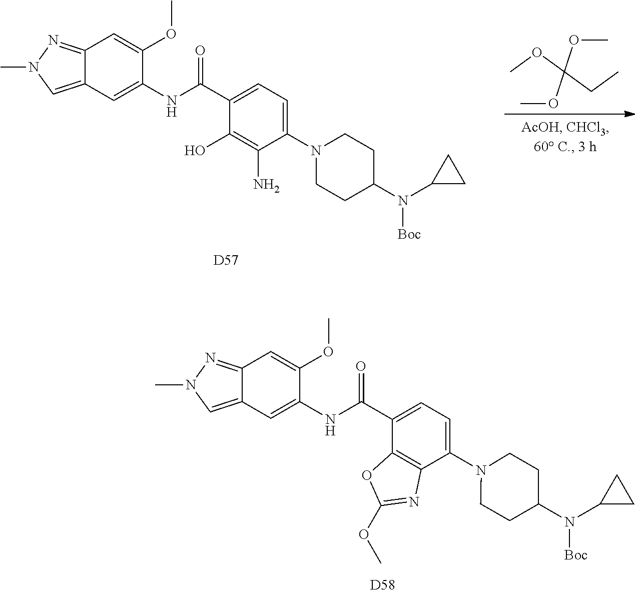

To a stirred mixture of Intermediate D57 (280.0 mg, 508.5 μmol) and 1,1,1-trimethoxypropane (136.4 mg, 1.02 mmol) in CHCl₃ (3 mL) was added AcOH (122.0 mg, 2.03 mmol) in portions at room temperature. The resulting mixture was stirred for 3 h at 60° C., then acidified to pH 8 with saturated NaHCO₃ (aq.), and subsequently extracted with EtOAc (3×30 mL). The combined organic layers were washed with brine (30 mL), dried over anhydrous Na₂SO₄, and filtered. The filtrate was concentrated under reduced pressure and purified by silica gel column chromatography (50% EtOAc in PE) to afford tert-butyl N-cyclopropyl-N-[1-[2-methoxy-7-[(6-methoxy-2-methyl-indazol-5-yl)carbamoyl]-1,3-benzoxazol-4-yl]-4-piperidyl]-carbamate (D58, 270.0 mg, 457.1 μmol). LCMS (ES, m/z): 591 [M+H]⁺.

### Synthesis of Compound 679

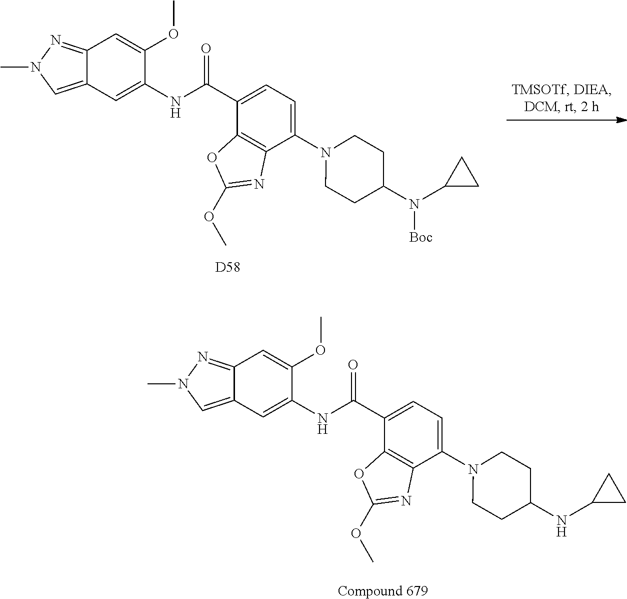

To a solution of Intermediate D58 (70.0 mg, 118.5 μmol) in DCM (0.6 mL) were added DIEA (30.6 mg, 237.0 μmol) and TMSOTf (79.0 mg, 355.5 μmol), and the reaction was stirred for 2 h at room temperature. The resulting mixture was concentrated under reduced pressure and purified by reversed-phase flash chromatography (Condition 3, Gradient 20) to afford 4-[4-(cyclopropylamino)-1-piperidyl]-2-methoxy-N-(6-methoxy-2-methyl-indazol-5-yl)-1,3-benzoxazole-7-carboxamide (Compound 679, 10.9 mg, 21.9 μmol). LCMS (ES, m/z): 491 [M+H]⁺. ¹H NMR (400 MHz, DMSO-d₆) δ 9.46 (s, 1H), 8.65 (s, 1H), 8.22 (s, 1H), 7.72 (d, J=8.8 Hz, 1H), 7.10 (s, 1H), 6.84 (d, J=8.9 Hz, 1H), 4.30-4.26 (m, 2H), 4.25 (s, 3H), 4.09 (s, 3H), 3.99 (s, 3H), 3.06 (t, J=11.7 Hz, 2H), 2.75 (s, 1H), 2.50 (s, 1H), 2.34 (s, 0H), 2.12 (dt, J=6.6, 3.2 Hz, 1H), 1.96 (d, J=12.8 Hz, 2H), 1.40 (q, J=10.7 Hz, 2H), 0.39 (dt, J=6.3, 3.0 Hz, 2H), 0.22 (p, J=3.9 Hz, 2H).

An analogous method was followed to obtain the following compounds.

| | Starting | |
|---|---|---|
| Compound | Material | Characterization |
| 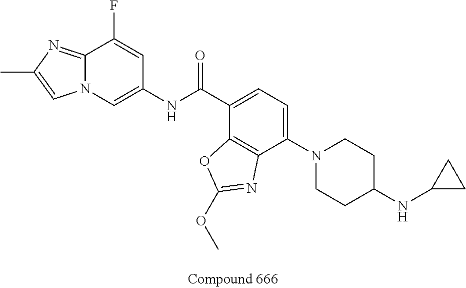 [SMILES: COc1nc2c(N3CCC(NC4CC4)CC3)ccc(C(=O)Nc3cc(F)c4nc(C)cn4c3)c2o1] | 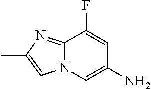 | Modification: Step 2 was ran at room temperature for 4 h. Step 4 was ran with tetramethoxymethane. The residue was purified by reversed-phase flash chromatography (Condition 3, Gradient 39) to afford 4-[4-(cyclopropylamino)-1- piperidyl]-N-(8-fluoro-2-methyl- imidazo[1,2-a]-pyridin-6-yl)-2-methoxy- 1,3-benzoxazole-7-carboxamide (Com- pound 666, 15 mg, 31.4 μmol). LCMS (ES, m/z): 479 [M + H]⁺. ¹H NMR (400 MHz, DMSO-d₆) δ 10.04 (s, 1H), 9.08 (d, J = 1.6 Hz, 1H), 7.91 (d, J = 3.1 Hz, 1H), 7.57 (d, J = 8.8 Hz, 1H), 7.27 (dd, J = 12.7, 1.6 Hz, 1H), 6.81 (d, J = 8.8 Hz, 1H), 4.25 (d, J = 12.8 Hz, 2H), 4.20 (s, 3H), 3.05 (t, J = 11.7 Hz, 2H), 2.74 (d, J = 10.6 Hz, 1H), 2.34 (s, 3H), 2.23 (s, |
| | | 1H), 2.12 (dq, J = 6.7, 3.4 Hz, 1H), |
| | | 1.95 (d, J = 11.9 Hz, 2H), 1.41 (q, J = 9.9 |
| | | Hz, 2H), 0.43-0.35 (m, 2H), 0.22 (dt, |
| | | J = 6.4, 3.9 Hz, 2H). |
| 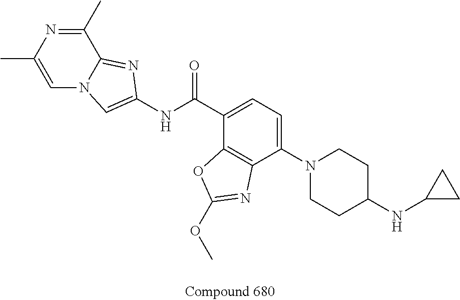 [SMILES: COc1nc2c(N3CCC(NC4CC4)CC3)ccc(C(=O)Nc3cn4cc(C)nc(C)c4n3)c2o1] | 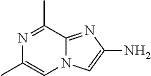 | Modifications: Step 2 was ran at room temperature for 8 h. Step 3 was ran for 3 h. Step 4 was ran using tetramethoxy- methane. The residue was purified by reversed-phase flash chromatography (Condition 3, Gradient 11) to afford 4- (4-(cyclopropylamino)-piperidin-1-yl)- N-(6,8-dimethylimidazo-[1,2-a]-pyrazin- 2-yl)-2-methoxy-benzo[d]-oxazole-7- carboxamide. (Compound 680, 70 mg, 14.7 μmol). LCMS (ES, m/z): 476 [M + H]⁺. ¹H NMR (400 MHz, DMSO- d₆) δ 10.64 (s, 1H), 8.31 (s, 1H), 8.29 (s, 1H), 7.69 (d, J = 8.8 Hz, 1H), 6.77 (d, J = 8.9 Hz, 1H), 4.27 (d, J = 12.9 Hz, 2H), 4.22 (s, 3H), 3.10-2.99 (m, 2H), 2.80-2.71 (m, 1H), 2.69 (s, 3H), 2.40- 2.35 (m, 3H), 2.13 (tt, J = 6.8, 3.7 Hz, 1H), 1.96 (d, J = 12.2 Hz, 2H), 1.42-1.39 |
| | | (m, 2H), 0.39 (td, J = 6.4, 4.1 Hz, 2H), |
| | | 0.27-0.19 (m, 2H). |

### Example 208: Synthesis of Compound 681

### Synthesis of Intermediate D59

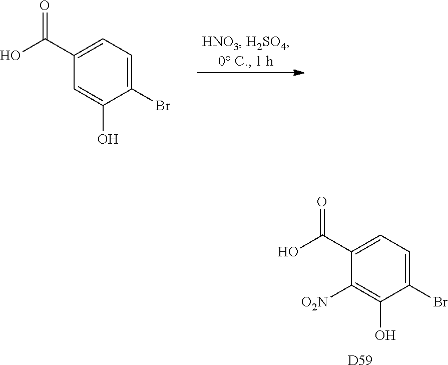 [SMILES: O=C(O)c1ccc(Br)c(O)c1 ; "HNO3, H2SO4,0° C., 1 h" ; O=C(O)c1ccc(Br)c(O)c1[N+](=O)[O-] ; "D59"]

A mixture of 4-bromo-3-hydroxybenzoic acid (5.00 g, 23.1 mmol) in sulfuric acid (100 mL) was stirred at 20° C. for 30 min, then cooled to 0° C., and treated with a chilled (cooled with an ice bath) solution of fuming nitric acid (1.16 mL, 27.6 mmol) dropwise. The reaction mixture was stirred at 0° C. for 1 h, quenched by pouring over ice, and extracted with EtOAc (2×200 mL). The combined organic extracts were washed with water (2×200 mL) and brine (200 mL), dried over anhydrous Na₂SO₄, filtered, and concentrated to yield 4-bromo-3-hydroxy-2-nitrobenzoic acid (D59, 4.5 g, 17.2 mmol). LCMS (ES, m/z): 260 [M−H].

### Synthesis of Intermediate D60

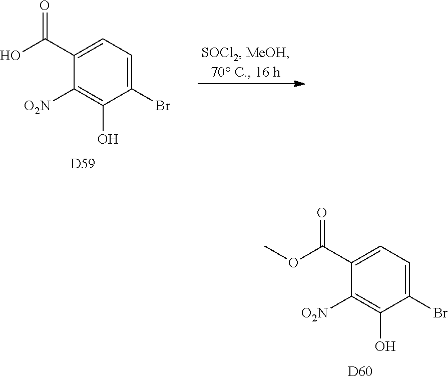

To a stirred mixture of Intermediate D59 (4.5 g, 17.2 mmol) in MeOH (100 mL) was added thionyl chloride (8.13 g, 69 mmol) dropwise at 0° C., and the reaction was stirred for 16 h at 70° C. The resulting mixture was concentrated under reduced pressure and purified by silica gel column chromatography (25% EtOAc in PE) to afford methyl 4-bromo-3-hydroxy-2-nitrobenzoate (D60, 4.5 g, 16.4 mmol). LCMS (ES, m/z): 274 [M−H].

### Synthesis of Intermediate D61

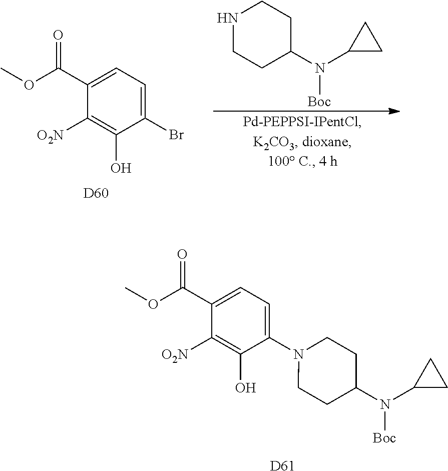

To a stirred mixture of Intermediate D60 (700 mg, 2.5 mmol) in dioxane (7 mL) were added tert-butyl N-cyclopropyl-N-(4-piperidyl)carbamate (731.3 mg, 3.04 mmol), K₂CO₃ (700.9 mg, 5.07 mmol) and Pd-PEPPSI-IPentCl (213.1 mg, 253.6 μmol) at room temperature under N₂. The mixture was stirred for 4 h at 100° C., then diluted with H₂O (20 mL), and subsequently extracted with DCM (2×20 mL). The combined organic layers were washed with H₂O (20 mL), dried over anhydrous Na₂SO₄, and filtered. The filtrate was concentrated under reduced pressure and purified by silica gel column chromatography (25% EtOAc in PE) to afford methyl 4-[4-[tert-butoxycarbonyl(cyclopropyl)amino]-1-piperidyl]-3-hydroxy-2-nitro-benzoate (D61, 390 mg, 895.6 μmol). LCMS (ES, m/z): 436 [M+H]⁺.

### Synthesis of Intermediate D62

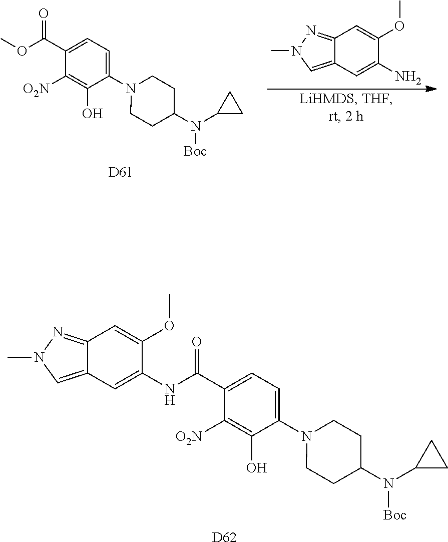

To a solution of methyl Intermediate D61 (120 mg, 275.6 μmol) in THF (1.2 mL) was added 6-methoxy-2-methyl-indazol-5-amine (68.3 mg, 385.8 μmol) at room temperature. Then LiHMDS (1 M, 1.1 mL) was added dropwise at 0° C. under N₂. The mixture was stirred for 2 h at room temperature under N₂, then quenched with NH₄Cl (aq.), and subsequently extracted with EtOAc (2×15 mL). The combined organic layers were washed with H₂O (20 mL), dried over anhydrous Na₂SO₄, and filtered. The filtrate was concentrated under reduced pressure and purified by silica gel column chromatography (40% EtOAc in DCM) to afford tert-butyl N-cyclopropyl-N-[1-[2-hydroxy-4-[(6-methoxy-2-methyl-indazol-5-yl)carbamoyl]-3-nitro-phenyl]-4-piperidyl]carbamate (D62, 80 mg, 137.8 μmol). LCMS (ES, m/z): 581 [M+H]⁺.

### Synthesis of Intermediate D63

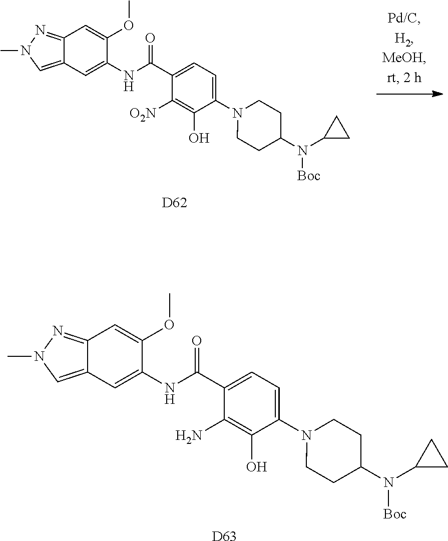 [SMILES: COc1cc2nn(C)cc2cc1NC(=O)c1ccc(N2CCC(N(C(=O)OC(C)(C)C)C3CC3)CC2)c(O)c1[N+](=O)[O-] ; COc1cc2nn(C)cc2cc1NC(=O)c1ccc(N2CCC(N(C(=O)OC(C)(C)C)C3CC3)CC2)c(O)c1N ; "D62" ; "D63"]

To a solution of Intermediate D62 (80 mg, 137.8 μmol) in MeOH (10 mL) was added Pd/C (10% on Carbon (wetted with ca. 55% Water), 29 mg) at room temperature under N₂. The mixture was stirred for 2 h at room temperature under H₂, then filtered through a Celite pad and concentrated under reduced pressure to afford tert-butyl N-[1-[3-amino-2-hydroxy-4-[(6-methoxy-2-methyl-indazol-5-yl)carbamoyl]phenyl]-4-piperidyl]-N-cyclopropyl-carbamate (D63, 60 mg, 109 μmol). LCMS (ES, m/z): 551 [M+H]⁻.

### Synthesis of Intermediate D64

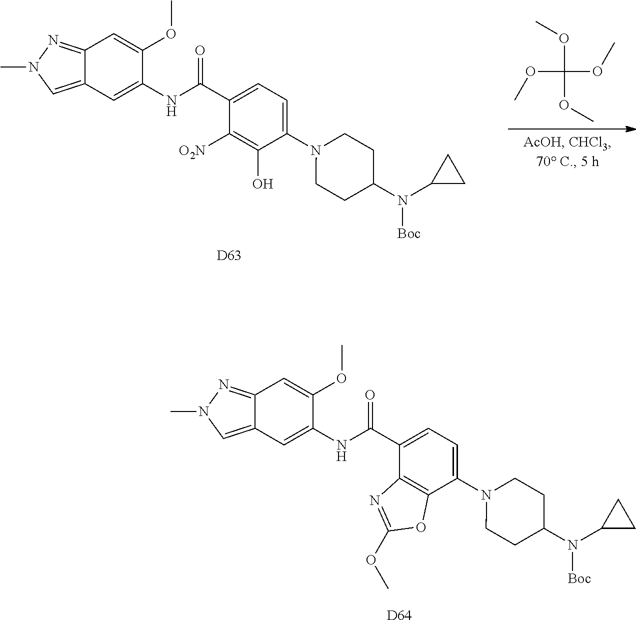

To a solution of Intermediate D63 (55 mg, 99.9 μmol) in CHCl₃ (1 mL) were added tetramethoxymethane (27.2 mg, 199.8 μmol) and acetic acid (24.0 mg, 399.5 μmol). The mixture was stirred for 5 h at 70° C., then diluted with H₂O and adjusted to pH 8 with NaHCO₃ (aq.), and subsequently extracted with DCM (2×10 mL). The combined organic layers were washed with H₂O (10 mL), dried over anhydrous Na₂SO₄, and filtered. The filtrate was concentrated under reduced pressure and purified by silica gel column chromatography (60% EtOAc in PE) to afford tert-butyl N-cyclopropyl-N-[1-[2-methoxy-4-[(6-methoxy-2-methyl-indazol-5-yl)-carbamoyl]-1,3-benzoxazol-7-yl]-4-piperidyl]carbamate (D64, 55 mg, 93.1 μmol). LCMS (ES, m/z): 591 [M+H]⁺.

### Synthesis of Compound 681

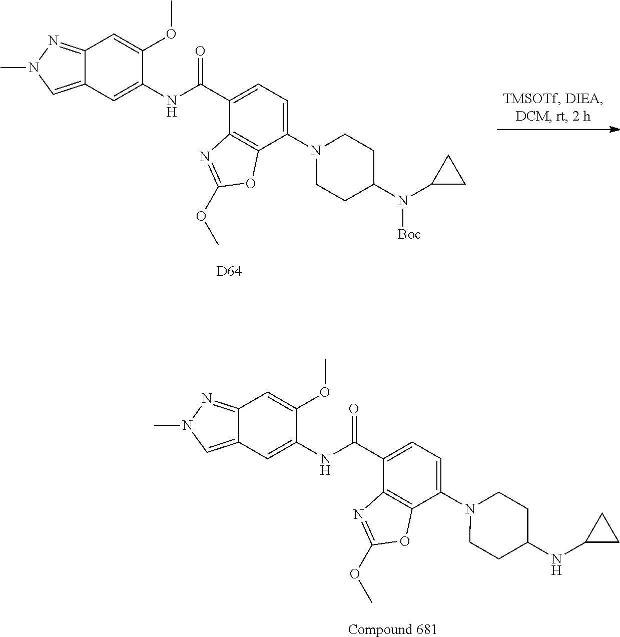

To a solution of Intermediate D64 (50 mg, 84.7 μmol) in DCM (0.5 mL) were added DIEA (21.9 mg, 169.3 μmol) and TMSOTf (56.4 mg, 253.9 μmol), and the reaction was stirred for 2 h at room temperature. The resulting mixture was concentrated under reduced pressure and purified by Prep-HPLC (Condition 5, Gradient 4) to afford 7-[4-(cyclopropylamino)-1-piperidyl]-2-methoxy-N-(6-methoxy-2-methyl-indazol-5-yl)-1,3-benzoxazole-4-carboxamide (Compound 681, 22 mg, 44.9 μmol). LCMS (ES, m/z): 491 [M+H]′. ¹H NMR (400 MHz, DMSO-d₆) δ 10.94 (s, 1H), 8.81 (s, 1H), 8.17 (s, 1H), 7.85 (d, J=8.8 Hz, 1H), 7.07 (s, 1H), 6.85 (d, J=8.9 Hz, 1H), 4.37 (s, 3H), 4.07 (s, 3H), 3.95 (s, 3H), 3.86 (d, J=12.9 Hz, 2H), 3.07-2.96 (m, 2H), 2.73 (dq, J=9.8, 5.9, 4.8 Hz, 1H), 2.20 (s, 1H), 2.09 (tt, J=6.7, 3.6 Hz, 1H), 1.94 (d, J=11.4 Hz, 2H), 1.47-1.30 (m, 2H), 0.41-0.32 (m, 2H), 0.24-0.16 (m, 2H).

An analogous method was followed to obtain the following compounds.

| | Starting | |
|---|---|---|
| Compound | Material | Characterization |
| 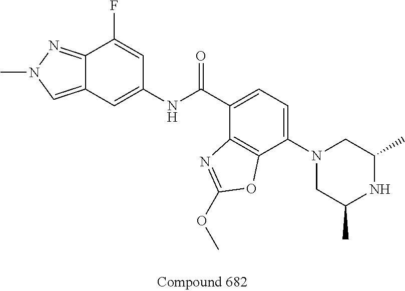 | 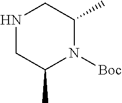 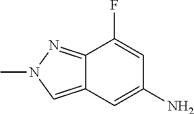 | Modifications: Step 4 was ran for 3 h. Step 5 was ran for 3 h. Step 6 was ran at 60° C. for 3 h. The crude product was purified by Prep- HPLC (Condition 5, Gradient 19b) to afford 7-[(3S,5S)-3,5-dimethylpiperazin-1- yl]-N-(7-fluoro-2-methyl-indazol-5-yl)-2- methoxy-1,3-benzoxazole-4-carboxamide (Compound 682, 10 mg, 22.1 μmol). LCMS (ES, m/z): 453 [M + H]⁺. ¹H NMR (400 MHz, DMSO-d₆) δ 10.64 (s, 1H), 8.42 (d, J = 2.8 Hz, 1H), 8.06 (d, J = 1.6 Hz, 1H), 7.82 (d, J = 8.8 Hz, 1H), 7.37 (dd, J = 13.1, 1.7 Hz, 1H), 6.86 (d, J = 8.9 Hz, 1H), 4.38 (s, 3H), 4.19 (s, 3H), 3.40 (dd, J = 11.8, 3.2 Hz, 2H), 3.22 (td, J = 6.4, 3.2 Hz, 2H), 3.09 (dd, J = 11.8, 6.2 Hz, 2H), 2.25-2.22 (m, 1H), 1.11 (d, J = 6.5 Hz, 6H). |

### Example 209: Synthesis of Compound 488

### Synthesis of Intermediate D65

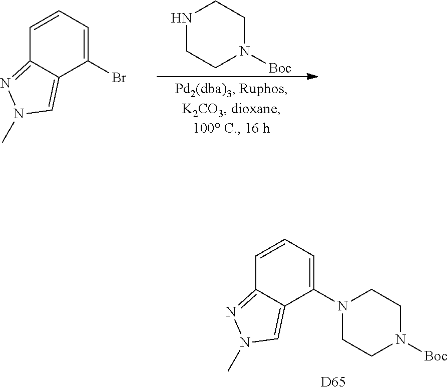 [SMILES: Cn1cc2c(Br)cccc2n1 ; CC(C)(C)OC(=O)N1CCNCC1 ; Cn1cc2c(N3CCN(C(=O)OC(C)(C)C)CC3)cccc2n1 ; "D65"]

To a stirred mixture of 4-bromo-2-methyl-indazole (2.1 g, 9.95 mmol) and tert-butyl piperazine-1-carboxylate (1.85 g, 9.95 mmol) in dioxane (40 mL) were added Pd₂(dba)₃ (911 mg, 995 μmol), K₂CO₃ (2.75 g, 19.9 mmol) and Ruphos (928 mg, 1.99 mmol), and the reaction was stirred for 16 h at 100° C. under N₂. The resulting mixture was allowed to cool to room temperature, diluted with water (40 mL), and extracted with EtOAc (2×50 mL). The combined organic layers were washed with brine (100 mL), dried over anhydrous Na₂SO₄, and filtered. The filtrate was concentrated under reduced pressure and purified by silica gel column chromatography (20% EtOAc in PE) to afford tert-butyl 4-(2-methylindazol-4-yl) piperazine-1-carboxylate (D65, 2.06 g, 6.5 mmol). LCMS (ES, m/z): 317 [M+H]⁺.

### Synthesis of Intermediate D66

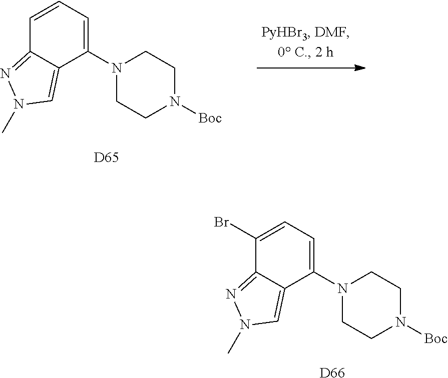 [SMILES: Cn1cc2c(N3CCN(C(=O)OC(C)(C)C)CC3)cccc2n1 ; "D65" ; Cn1cc2c(N3CCN(C(=O)OC(C)(C)C)CC3)ccc(Br)c2n1 ; "D66"]

To a solution of Intermediate D65 (2.3 g, 7.3 mmol) in DMF (25 mL) was added 2-(bromomethyl)pyridine hydrobromide (1.84 g, 7.3 mmol). The reaction was stirred for 2 h at 0° C., then diluted with water (50 mL) and extracted with EtOAc (2×50 mL). The combined organic layers were washed with brine (100 mL), dried over anhydrous Na₂SO₄, and filtered. The filtrate was concentrated under reduced pressure and purified by silica gel column chromatography (50% EtOAc in PE) to afford tert-butyl 4-(7-bromo-2H-indazol-4-yl) piperazine-1-carboxylate (D66, 1.3 g, 3.4 mmol). LCMS (ES, m/z): 395 [M+H]⁺.

### Synthesis of Intermediate D67

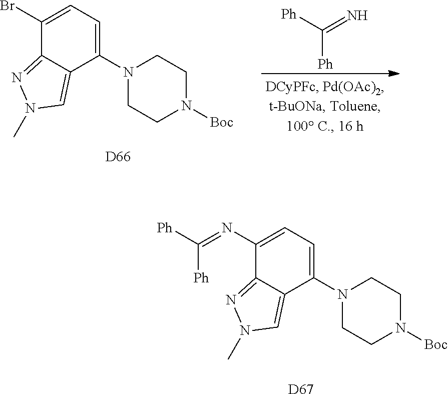 [SMILES: Cn1cc2c(N3CCN(C(=O)OC(C)(C)C)CC3)ccc(Br)c2n1 ; N=C(c1ccccc1)c1ccccc1 ; "D66" ; Cn1cc2c(N3CCN(C(=O)OC(C)(C)C)CC3)ccc(N=C(c3ccccc3)c3ccccc3)c2n1 ; "D67"]

To a stirred mixture of Intermediate D66 (1.89 g, 3.99 mmol) and diphenylmethanimine (3.61 g, 19.9 mmol) in toluene (20 mL) were added 1,1-Bis(dicyclohexylphosphino) ferrocene (485 mg, 797.2 μmol), Pd(AcO)₂ (89 mg, 398.6 μmol) and t-BuONa (1.15 g, 11.96 mmol) at room temperature under N₂. The resulting mixture was stirred for 16 h at 100° C. under N₂, then allowed to cool to room temperature, diluted with water (20 mL), and subsequently extracted with EtOAc (2×30 mL). The combined organic layers were washed with water (2×40 mL), brine (40 mL), dried over anhydrous Na₂SO₄, and filtered. The filtrate was concentrated under reduced pressure and purified by silica gel column chromatography (75% EtOAc in PE) to afford tert-butyl 4-[7-(benzhydrylideneamino)-2-methyl-indazol-4-yl]piperazine-1-carboxylate (D67, 1.1 g, 2.22 mmol). LCMS (ES, m/z): 496 [M+H]⁺.

### Synthesis of Intermediate D68

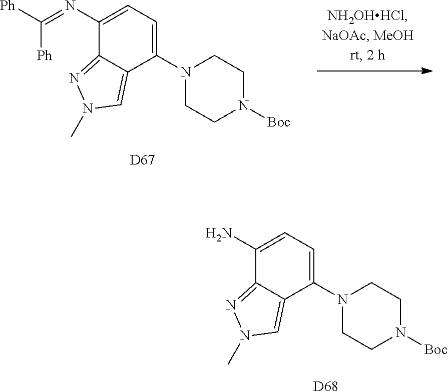 [SMILES: Cn1cc2c(N3CCN(C(=O)OC(C)(C)C)CC3)ccc(N=C(c3ccccc3)c3ccccc3)c2n1 ; "D67" ; Cn1cc2c(N3CCN(C(=O)OC(C)(C)C)CC3)ccc(N)c2n1 ; "D68"]

To a solution of tert-butyl Intermediate D67 (1.1 g, 2.22 mmol) in MeOH (20 mL) was added AcONa (546 mg, 6.66 mmol) and hydroxylamine hydrochloride (771 mg, 11.1 mmol). The reaction was stirred for 2 h at 25° C., then concentrated under reduced pressure and adjusted to pH 8 with saturated NaHCO₃ (aq.). The resulting mixture was diluted with water (10 mL) and extracted with EtOAc (2×20 mL). The combined organic layers were washed with water (20 mL) and brine (20 mL), dried over anhydrous Na₂SO₄, and filtered. The filtrate was concentrated under reduced pressure and purified by silica gel column chromatography (83% EtOAc in PE) to afford tert-butyl 4-(7-amino-2-methyl-indazol-4-yl) piperazine-1-carboxylate (D68, 400 mg, 1.21 mmol). LCMS (ES, m/z): 332 [M+H]⁺.

### Synthesis of Intermediate D69

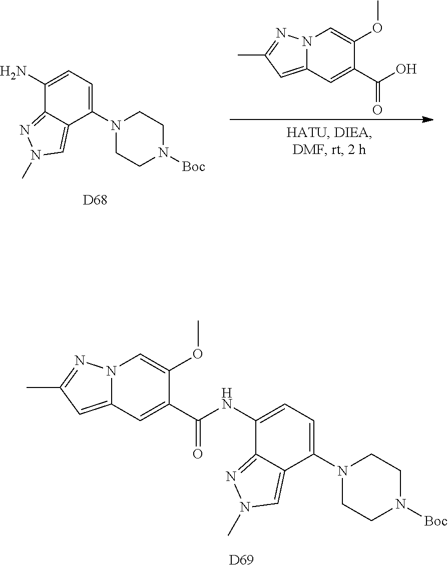

To a stirred mixture of Intermediate D68 (200 mg, 603.5 μmol) and 6-methoxy-2-methyl-pyrazolo[1,5-a]pyridine-5-carboxylic acid (137 mg, 663.8 μmol) in DMF (2 mL) were added DIEA (234 mg, 1.8 mmol) and HATU (275 mg, 724.2 μmol) at room temperature. The resulting mixture was stirred for 2 h at 25° C., then diluted with water (8 mL) and extracted with EtOAc (3×10 mL). The combined organic layers were washed with water (2×10 mL), brine (20 mL), dried over anhydrous Na₂SO₄, and filtered. The filtrate was concentrated under reduced pressure and purified by silica gel column chromatography (80% EtOAc in PE) to afford tert-butyl 4-[7-[(6-methoxy-2-methyl-pyrazolo[1,5-a]pyridine-5-carbonyl)amino]-2-methyl-indazol-4-yl]-piperazine-1-carboxylate (D69, 120 mg, 230.9 μmol). LCMS (ES, m/z): 520 [M+H]⁺.

### Synthesis of Compound 488

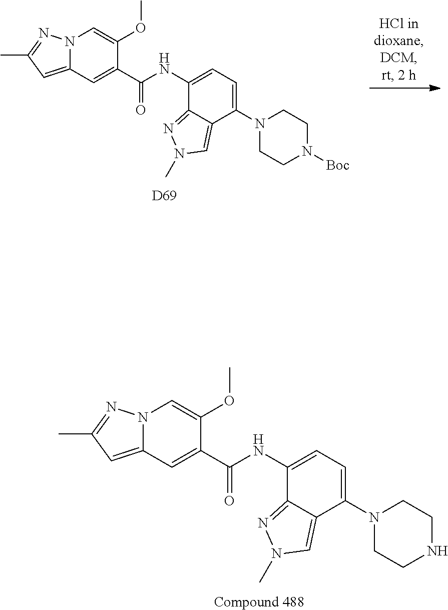

To a solution of Intermediate D69 (120 mg, 231 μmol) in DCM (2 mL) was added 4M HCl in 1,4-dioxane (1.2 mL), and the reaction was stirred for 2 h at 25° C. The resulting mixture was concentrated under reduced pressure and purified by reversed-phase flash chromatography (Condition 3, Gradient 38) to afford 6-methoxy-2-methyl-N-(2-methyl-4-piperazin-1-yl-indazol-7-yl) pyrazolo[1,5-a]pyridine-5-carboxamide (Compound 488, 22.6 mg, 53.9 μmol). LCMS (ES, m/z): 420 [M+H]⁺. ¹H NMR (300 MHz, DMSO-d₆) δ 10.87 (s, 1H), 8.59 (s, 1H), 8.48 (s, 1H), 8.33 (s, 1H), 8.05 (d, J=7.8 Hz, 1H), 6.61 (s, 1H), 6.30 (d, J=8.1 Hz, 1H), 4.21 (s, 3H), 4.17 (s, 3H), 3.16-3.01 (m, 4H), 2.98-2.80 (m, 4H), 2.41 (s, 3H).

An analogous method was followed to obtain the following compound.

| | Starting | |
|---|---|---|
| Compound | Material | Characterization |
| 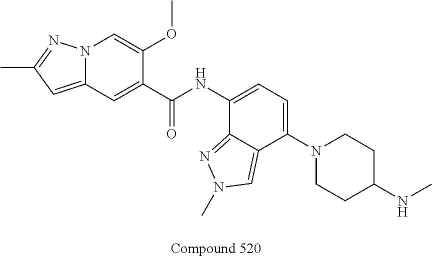 | 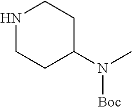 | Modifications: Step 4 was ran for 40 min. Step 5 was ran for 1 h. Step 6 was ran in EtOAc. The crude product was purified by Prep-HPLC (Condition 18, Gradient 1) to afford 6-methoxy- 2-methyl-N-[2-methyl-4-[4-(methyamino)-1- piperidyl]indazol-7-yl]-pyrazolo[1,5-a] pyridine-5-carboxamide. (Compound 520, 21.7 mg, 48.5 μmol). LCMS (ES, m/z): 448 [M + H]⁺. ¹H NMR (400 MHz, DMSO-d₆) δ 10.85 (s, 1H), 8.59 (s, 1H), 8.44 (s, 1H), 8.32 (s, 1H), 8.03 (d, J = 8.0 Hz, 1H), 6.60 (s, 1H), 6.30 (d, J = 8.0 Hz, 1H), 4.21 (s, 3H), 4.16 (s, 3H), 3.60-3.57 (m, 2H), 2.75-2.73 (m, 2H), 2.51-2.41 (m, 4H), 2.31 (s, 3H), 1.96-1.93 (m, 2H), 1.49- 1.40 (m, 2H). |

### Example 210: Synthesis of Compound 632

### Synthesis of Intermediate D70

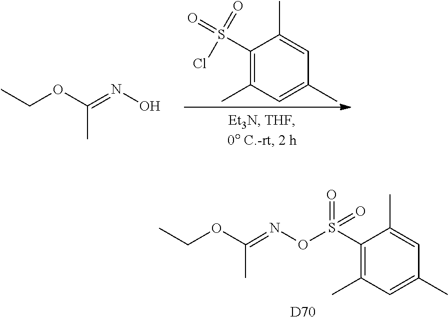

To a stirred mixture of ethyl (1E)-N-hydroxyethanimidate (100 g, 969.8 mmol) in THF (2 L) were added Et₃N (195.9 g, 1.94 mol) and 2,4,6-trimethylbenzenesulfonyl chloride (286.3 g, 1.31 mol) at room temperature under N₂. The resulting mixture was stirred for 2 h at rt under N₂, then diluted with water (2 μL), and subsequently extracted with EtOAc (3×1 L). The combined organic layers were washed with water (3×2 L) and brine (2 μL), dried over anhydrous Na₂SO₄, and filtered. The filtrate was concentrated under reduced pressure and purified by silica gel column chromatography (33% EtOAc in PE) to afford (1E)-N-(2,4,6-trimethylphenyl)-sulfonyloxyethanimidic acid (D70, 91 g, 353.7 mmol). LCMS (ES, m/z): 286 [M+H]⁺.

### Synthesis of Intermediate D71

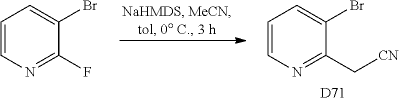

To a solution of MeCN (20.97 g, 511.4 mmol) in toluene (600 mL) was added NaHMDS (257 mL) at 0° C. The mixture was stirred for 15 min at 0° C. 3-bromo-2-fluoro-pyridine (30 g, 170.5 mmol) was added and the mixture was stirred for additional 3 h at 0° C. The reaction was quenched with NH₄Cl, then diluted with water (500 mL) and later extracted with EtOAc (3×500 mL). The combined organic layers were washed with water (3×1 L) and brine (1 μL), dried over anhydrous Na₂SO₄, and filtered. The filtrate was concentrated under reduced pressure and purified by silica gel column chromatography (33% EtOAc in PE) to afford 2-(3-bromo-2-pyridyl) acetonitrile (D71, 11 g, 55.8 mmol). LCMS (ES, m/z): 197 [M+H]⁺.

### Synthesis of Intermediate D72

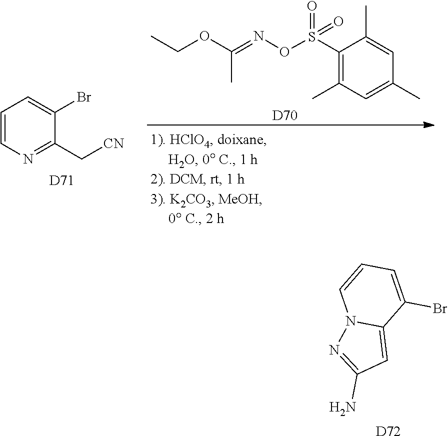

To a stirred mixture of Intermediate D70 (8.69 g, 30.5 mmol) in dioxane (26 mL) was added HClO₄ (5.4 mL) dropwise at 0° C., and the reaction was stirred for 1 h. The resulting mixture was diluted with ice water (10 mL). The precipitated solids were collected by filtration and washed with water (3×10 mL), dissolved in DCM (500 mL), and the solution was divided into two layers. The combined organic layers were dried over anhydrous MgSO₄, and filtered. The filtrate was cooled to 0° C., to which was added a solution of Intermediate D71 (4 g, 20.3 mmol) in DCM (120 mL), and the reaction was stirred at room temperature for 1 h. The resulting mixture was concentrated under reduced pressure and dissolved in MeOH (80 mL), followed by the addition of K₂CO₃ (8.7 g, 62.9 mmol) at 0° C. under an argon atmosphere. The mixture was stirred at 0° C. for 2 h, then quenched with water (200 mL) and extracted with EtOAc (200 mL). The organic layer was washed with water (200 mL) and brine (200 mL), dried over anhydrous MgSO₄, and filtered. The filtrate was concentrated under reduced pressure and purified by silica gel column chromatography (33% EtOAc in PE) to afford 4-bromopyrazolo[1,5-a]pyridin-2-amine (D72, 1.1 g, 5.19 mmol). LCMS (ES, m/z): 212 [M+H]⁺.

### Synthesis of Intermediate D74

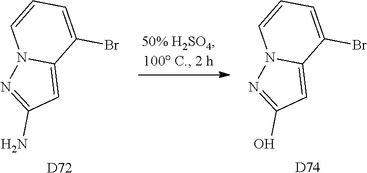

4-bromopyrazolo[1,5-a]pyridin-2-amine (3 g, 14.15 mmol) was dissolved in H₂SO₄ (50% aq.) (60 mL), and the reaction was stirred for 2 h at 100° C. The resulting mixture was cooled to rt and the solid was collected by filtration and washed with water (3×100 mL) to afford 4-bromopyrazolo[1,5-a]pyridin-2-ol (D74, 2.4 g, 11.3 mmol, 80% yield) as a solid. LCMS (ES, m/z): 213 [M+H]⁺.

### Synthesis of Intermediate D75

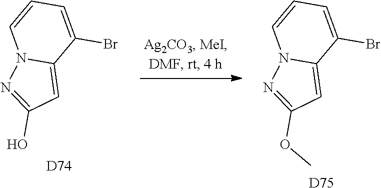

To a stirred mixture of 4-bromopyrazolo[1,5-a]pyridin-2-ol (D74, 2.4 g, 11.3 mmol) in THF (32 mL) were added MeOH (16 mL) and TMSCHN₂ (1.28 g, 11.3 mmol), and the reaction was stirred for 4 h at room temperature. The resulting mixture was diluted with water (50 mL) and extracted with EtOAc (3×50 mL). The combined organic layers were washed with water (3×100 mL) and brine (100 mL), dried over anhydrous Na₂SO₄, and filtered. The filtrate was concentrated under reduced pressure and purified by silica gel column chromatography (33% EtOAc in PE) to afford 4-bromo-2-methoxy-pyrazolo[1,5-a]pyridine (D75, 1.5 g, 6.61 mmol, 59% yield) as a solid. LCMS (ES, m/z): 227 [M+H]⁺.

### Synthesis of Intermediate D76

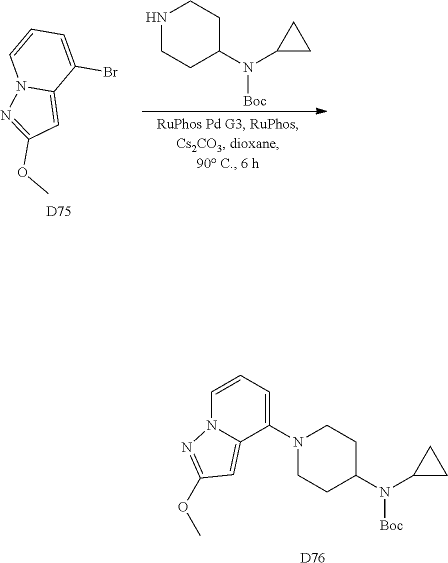

To a stirred mixture of 4-bromo-2-methoxy-pyrazolo[1,5-a]pyridine (D75, 1.5 g, 6.61 mmol) and tert-butyl N-cyclopropyl-N-(4-piperidyl)carbamate (1.91 g, 7.93 mmol) in dioxane (30 mL) were added Cs₂CO₃ (2.15 g, 6.61 mmol) and RuPhos (3.08 g, 6.61 mmol) and RuPhos Pd G3 (5.53 g, 6.61 mmol) at room temperature under N₂. The resulting mixture was stirred for 6 h at 90° C. under N₂. The resulting mixture was concentrated under reduced pressure and purified by silica gel column chromatography (50% EtOAc in PE) to afford tert-butyl N-cyclopropyl-N-[1-(2-methoxypyrazolo[1,5-a]pyridin-4-yl)-4-piperidyl]carbamate (D76, 1.9 g, 4.92 mmol, 74% yield) as a solid. LCMS (ES, m/z): 387 [M+H]⁺.

### Synthesis of Intermediate D77

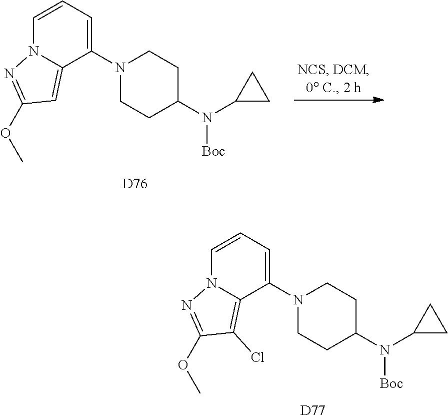 [SMILES: COc1cc2c(N3CCC(N(C(=O)OC(C)(C)C)C4CC4)CC3)cccn2n1 ; "D76" ; COc1nn2cccc(N3CCC(N(C(=O)OC(C)(C)C)C4CC4)CC3)c2c1Cl ; "D77"]

To a stirred mixture of Intermediate D76 (1.9 g, 4.92 mmol) in MeCN (40 mL) was added NCS (656.4 mg, 4.92 mmol) at 0° C. The resulting mixture was stirred for 2 h at 0° C. and then diluted with water (30 mL). The precipitated solids were collected by filtration and washed with water (3×30 mL) to afford tert-butyl N-[1-(3-chloro-2-methoxy-pyrazolo[1,5-a]pyridin-4-yl)-4-piperidyl]-N-cyclopropyl-carbamate (D77, 1.6 g, 3.8 mmol, 77% yield) as a solid. LCMS (ES, m/z): 421 [M+H]⁺.

### Synthesis of Intermediate D78

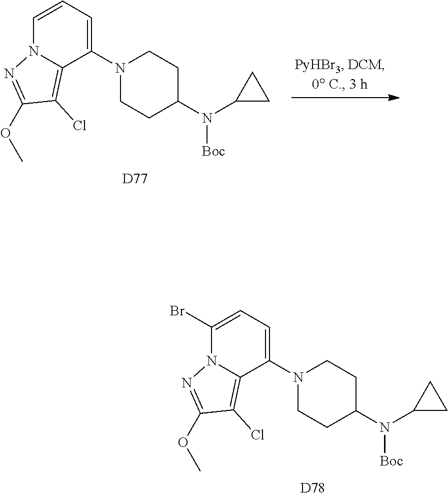 [SMILES: COc1nn2cccc(N3CCC(N(C(=O)OC(C)(C)C)C4CC4)CC3)c2c1Cl ; "D77" ; COc1nn2c(Br)ccc(N3CCC(N(C(=O)OC(C)(C)C)C4CC4)CC3)c2c1Cl ; "D78"]

To a stirred mixture of Intermediate D77 (1.6 g, 3.80 mmol) in DCM (32 mL) was added PyHBr₃ (1.15 g, 3.61 mmol) at 0° C. The resulting mixture was stirred for 3 h at 0° C., then diluted with water (50 mL), and subsequently extracted with DCM (3×50 mL). The combined organic layers were washed with water (3×100 mL) and brine (100 mL), dried over anhydrous Na₂SO₄, and filtered. The filtrate was concentrated under reduced pressure and purified by silica gel column chromatography (50% EtOAc in PE) to afford tert-butyl N-[1-(7-bromo-3-chloro-2-methoxy-pyrazolo[1,5-a]pyridin-4-yl)-4-piperidyl]-N-cyclopropyl-carbamate (D78, 1.3 g, 2.60 mmol, 68% yield) as a solid. LCMS (ES, m/z): 499 [M+H]⁺.

### Synthesis of Intermediate D79

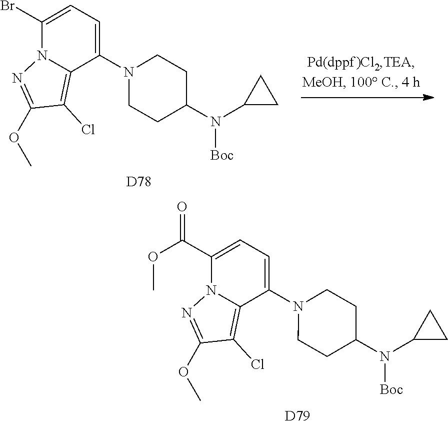 [SMILES: COc1nn2c(Br)ccc(N3CCC(N(C(=O)OC(C)(C)C)C4CC4)CC3)c2c1Cl ; "D78" ; COC(=O)c1ccc(N2CCC(N(C(=O)OC(C)(C)C)C3CC3)CC2)c2c(Cl)c(OC)nn12 ; "D79"]

To a solution of Intermediate D78 (1.3 g, 2.6 mmol) in MeOH (30 mL) were added TEA (788.1 mg, 7.8 mmol) and Pd(dppf)Cl₂ (212.2 mg, 260.1 μmol) in a pressure tank. The mixture was purged with N₂ for 3 min and then pressurized to 1 MPa with carbon monoxide at 100° C. for 4 h. The reaction mixture was cooled to room temperature and filtered to remove insoluble solids. The resulting mixture was concentrated under reduced pressure and purified by silica gel column chromatography (33% EtOAc in PE) to afford methyl 4-[4-[tert-butoxycarbonyl(cyclopropyl)amino]-1-piperidyl]-3-chloro-2-methoxy-pyrazolo[1,5-a]pyridine-7-carboxylate (D79, 1.0 g, 2.09 mmol, 81% yield) as a solid. LCMS (ES, m/z): 479 [M+H]⁺.

### Synthesis of Intermediate D80

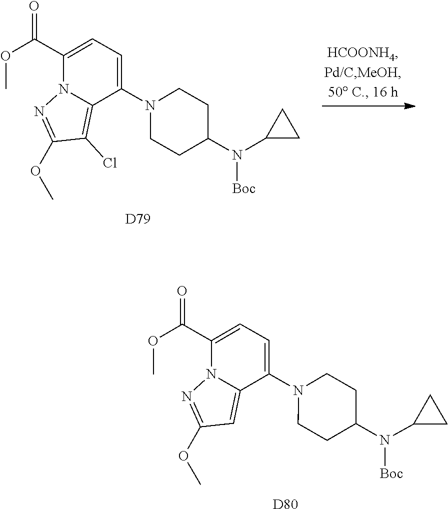 [SMILES: COC(=O)c1ccc(N2CCC(N(C(=O)OC(C)(C)C)C3CC3)CC2)c2c(Cl)c(OC)nn12 ; "D79" ; COC(=O)c1ccc(N2CCC(N(C(=O)OC(C)(C)C)C3CC3)CC2)c2cc(OC)nn12 ; "D80"]

To a stirred mixture of Intermediate D79 (1 g, 2.09 mmol) in MeOH (20 mL) were added HCOONH₄ (263.3 mg, 4.18 mmol) and Pd/C (200 mg) at room temperature under N₂. The resulting mixture was stirred for 16 h at 50° C. under N₂ and filtered. The filtrate was diluted with water (80 mL) and subsequently extracted with EtOAc (3×50 mL). The combined organic layers were washed with water (3×100 mL) and brine (100 mL), dried over anhydrous Na₂SO₄, and filtered. The filtrate was concentrated under reduced pressure and purified by silica gel column chromatography (50% EtOAc in PE) into afford methyl 4-[4-[tert-butoxycarbonyl-(cyclopropyl)amino]-1-piperidyl]-2-methoxy-pyrazolo-[1,5-a]pyridine-7-carboxylate (D80, 660 mg, 1.48 mmol, 71% yield) as a solid. LCMS (ES, m/z): 445 [M+H]⁺.

### Synthesis of Intermediate D81

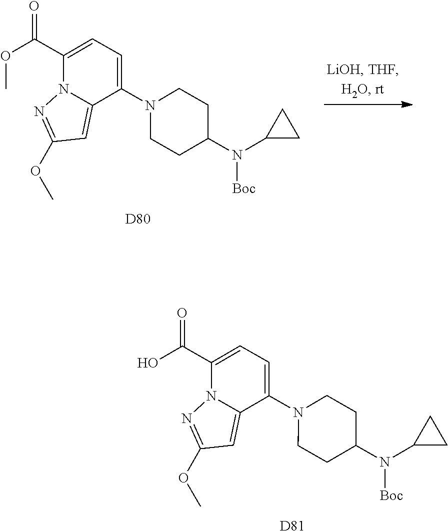 [SMILES: COC(=O)c1ccc(N2CCC(N(C(=O)OC(C)(C)C)C3CC3)CC2)c2cc(OC)nn12 ; "D80" ; COc1cc2c(N3CCC(N(C(=O)OC(C)(C)C)C4CC4)CC3)ccc(C(=O)O)n2n1 ; "D81"]

To a stirred mixture of Intermediate D80 (660 mg, 1.48 mmol) in THF (6 mL) and H₂O (6 mL) was added LiOH (177.8 mg, 7.42 mmol) at room temperature. The resulting mixture was stirred for 2 h, then acidified to pH 5 with HCl (2 N), and subsequently extracted with EtOAc (3×15 mL). The combined organic layers were washed with water (3×30 mL), dried over anhydrous Na₂SO₄, and filtered. The filtrate was concentrated under reduced pressure to afford 4-[4-[tert-butoxycarbonyl(cyclopropyl)amino]-1-piperidyl]-2-methoxy-pyrazolo[1,5-a]pyridine-7-carboxylic acid (D81, 600 mg, 1.39 mmol, 94% yield) as a solid. LCMS (ES, m/z): 431 [M+H]⁺.

### Synthesis of Intermediate D82

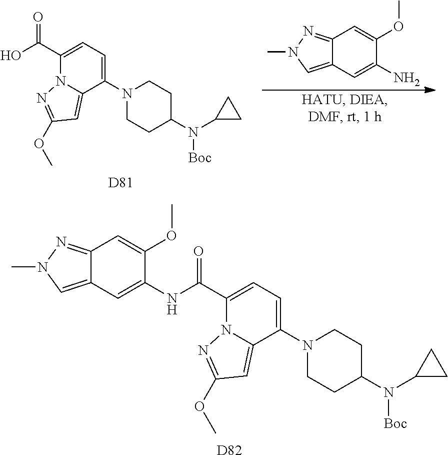

To a stirred mixture of Intermediate D81 (70 mg, 162.6 μmol) and 6-methoxy-2-methyl-indazol-5-amine (34.6 mg, 195.1 μmol) in DMF (1.4 mL) were added DIEA (63.1 mg, 487.8 μmol) and HATU (92.7 mg, 243.9 μmol), and the reaction was stirred for 2 h at room temperature. The resulting mixture was diluted with water (5 mL), and the precipitated solids were collected by filtration, washed with water (3×10 mL), and dried to afford tert-butyl N-cyclopropyl-N-[1-[2-methoxy-7-[(6-methoxy-2-methyl-indazol-5-yl)carbamoyl]pyrazolo[1,5-a]-pyridin-4-yl]-4-piperidyl]carbamate (D82, 80 mg, 135.7 μmol, 83% yield) as a solid. LCMS (ES, m/z): 590 [M+H]⁺.

### Synthesis of Compound 632

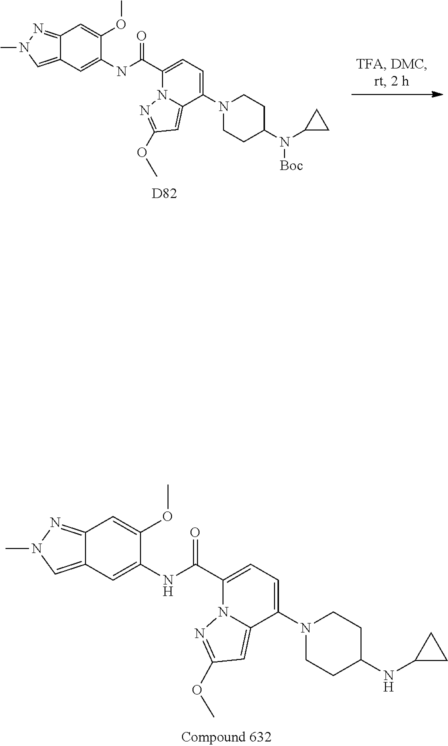

To a stirred mixture of Intermediate D82 (80 mg, 135.7 μmol) in DCM (1.6 mL) was added TFA (0.4 mL) dropwise, and the reaction was stirred for 2 h at room temperature. The resulting mixture was concentrated under reduced pressure and purified by Prep-HPLC (Condition 5, Gradient 29) to afford 4-[4-(cyclopropylamino)-1-piperidyl]-2-methoxy-N-(6-methoxy-2-methyl-indazol-5-yl) pyrazolo[1,5-a]pyridine-7-carboxamide (Compound 632, 33 mg, 67.4 μmol, 50% yield) as a solid. LCMS (ES, m/z): 490 [M+H]⁺. ¹H NMR (400 MHz, DMSO-d₆) δ 12.55 (s, 1H), 8.82 (s, 1H), 8.23 (s, 1H), 7.76 (d, J=8.2 Hz, 1H), 7.11 (s, 1H), 6.78 (d, J=8.2 Hz, 1H), 6.22 (s, 1H), 4.11 (d, J=7.9 Hz, 6H), 3.96 (s, 3H), 3.65 (d, J=12.4 Hz, 2H), 2.88 (t, J=11.3 Hz, 2H), 2.79-2.70 (m, 1H), 2.33 (s, 1H), 2.12 (tt, J=6.7, 3.6 Hz, 1H), 2.04-1.96 (m, 2H), 1.51 (q, J=10.1 Hz, 2H), 0.40 (dt, J=6.2, 3.0 Hz, 2H), 0.28-0.20 (m, 2H).

An analogous method was followed to obtain the following compounds.

| | Starting | |
|---|---|---|
| Compound | Material | Characterization |
| 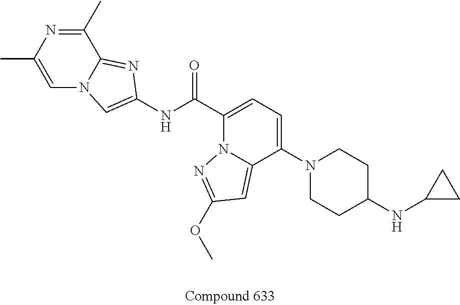 [SMILES: COc1cc2c(N3CCC(NC4CC4)CC3)ccc(C(=O)Nc3cn4cc(C)nc(C)c4n3)n2n1] | 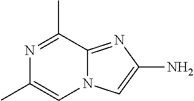 | The crude product was purified by Prep-HPLC (Condition 5, Gradient 30) to afford 4-[4- (cyclopropyl-amino)-1-piperidyl]- N-(6,8-dimethyl-imidazo[1,2-a] pyrazin-2-yl)-2-methoxypyrazolo [1,5-a]-pyridine-7-carboxamide (Compound 633, 40 mg, 84.3 μmol). LCMS (ES, m/z): 475 [M + H]⁺. ¹H NMR (400 MHz, DMSO-d₆) δ 12.81 (s, 1H), 8.32 (s, 2H), 7.76 (d, J = 8.1 Hz, 1H), 6.79 (d, J = 8.2 Hz, 1H), 6.30 (s, 1H), 4.07 (s, 3H), 3.68 (d, J = 12.0 Hz, 2H), 2.91 (t, J = 11.5 Hz, 2H), 2.76 (s, 1H), 2.69 (s, 3H), 2.37 (s, 4H), 2.13 (dt, J = 6.7, 3.2 Hz, 1H), 2.01 (d, J = 12.6 Hz, 2H), 1.52 (q, J = 11.1 Hz, 2H), 0.40 (dt, J = 6.3, 3.1 Hz, 2H), 0.25 (p, J = 3.9 Hz, 2H). |

### Example 211: Synthesis of D88

### Synthesis of Intermediate D83

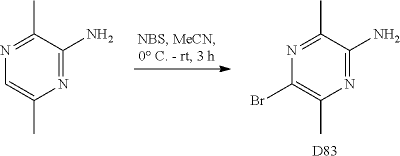

A solution of 3,6-dimethylpyrazin-2-amine (5 g, 40.6 mmol) in MeCN (50 mL) was treated with NBS (7.23 g, 40.6 mmol) at 0° C. under N₂. The resulting mixture was stirred for 3 h at room temperature under N₂, then diluted with water (60 mL) and subsequently extracted with EtOAc (3×50 mL). The combined organic layers were washed with brine (100 mL), dried over anhydrous Na₂SO₄, filtered, and concentrated under reduced pressure to afford 5-bromo-3,6-dimethyl-pyrazin-2-amine (D83, 6.8 g, 33.7 mmol). LCMS (ES, m/z): 202 [M+H]⁻.

### Synthesis of Intermediate D84

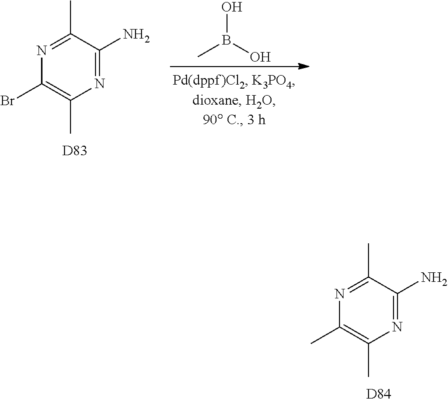

To a stirred mixture of methylboronic acid (3.91 g, 65.3 mmol) and 5-bromo-3,6-dimethyl-pyrazin-2-amine (D83, 6.6 g, 32.7 mmol) in dioxane (70 mL) and H₂O (10 mL) were added K₃PO₄ (20.8 g, 98 mmol) and Pd(dppf)Cl₂ (2.39 g, 3.3 mmol) at room temperature under N₂. The resulting mixture was stirred for 3 h at 90° C. under N₂, then allowed to cool to room temperature and diluted with water (70 mL), and subsequently extracted with EtOAc (2×100 mL). The combined organic layers were washed with brine (100 mL), dried over anhydrous Na₂SO₄, and filtered. The filtrate was concentrated under reduced pressure and purified by silica gel column chromatography (33% EtOAc in PE) to afford 3,5,6-trimethylpyrazin-2-amine (D84, 3.6 g, 26.2 mmol). LCMS (ES, m/z): 138 [M+H]⁺.

### Synthesis of Intermediate D85

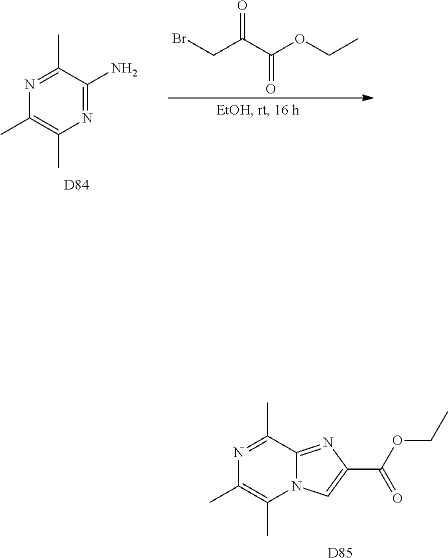

A solution of 3,5,6-trimethylpyrazin-2-amine (D84, 3.2 g, 23.3 mmol) in EtOH (40 mL) was treated with ethyl 3-bromo-2-oxo-propanoate (6.8 g, 35 mmol) at room temperature, stirred for 16 h, then diluted with water (60 mL), and subsequently extracted with EtOAc (3×50 mL). The combined organic layers were washed with brine (60 mL), dried over anhydrous Na₂SO₄, and filtered. The filtrate was concentrated under reduced pressure and purified by silica gel column chromatography (50% EtOAc in PE) to afford ethyl 5,6,8-trimethylimidazo[1,2-a]pyrazine-2-carboxylate (D85, 2.9 g, 12.4 mmol). LCMS (ES, m/z): 234 [M+H]⁺.

### Synthesis of Intermediate D86

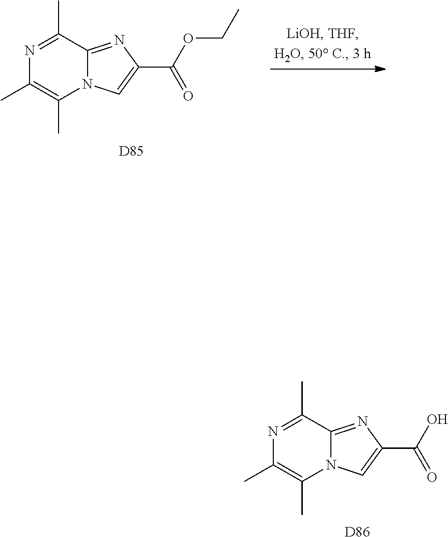

A solution of ethyl Intermediate D85 (2.8 g, 12.0 mmol) in THF (28 mL) and H₂O (28 mL) was treated with LiOH·H₂O (2.52 g, 60.0 mmol), and the reaction was stirred for 3 h at 50° C. The mixture was purified by Prep-HPLC (Condition 5, Gradient 26) to afford 5,6,8-trimethylimidazo[1,2-a]pyrazine-2-carboxylic acid (D86, 2 g, 9.75 mmol). LCMS (ES, m/z): 206 [M+H]⁺.

### Synthesis of Intermediate D87

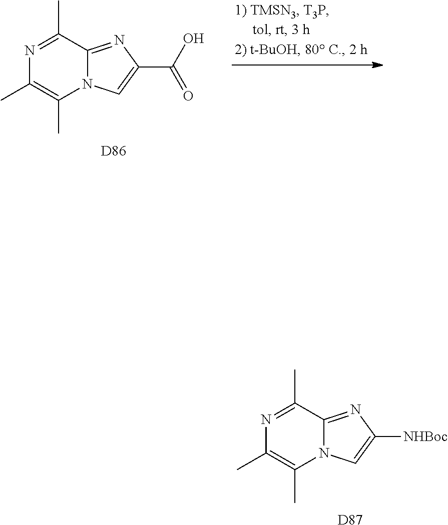

To a stirred mixture of Intermediate D86 (500 mg, 2.4 mmol) and TMSN₃ (700.4 mg, 6.1 mmol) in toluene (5 mL) was added T₃P (1.94 g, 6.1 mmol), and the reaction was stirred for 3 h at room temperature. To the above mixture was added t-BuOH (2 mL) at room temperature, then stirred for additional 2 h at 80° C., later diluted with water (10 mL), and subsequently extracted with EtOAc (3×30 mL). The combined organic layers were washed with water (2×40 mL) and brine (40 mL), dried over anhydrous Na₂SO₄, and filtered. The filtrate was concentrated under reduced pressure and purified by silica gel column chromatography (50% EtOAc in PE) to afford tert-butyl N-(5,6,8-trimethylimidazo[1,2-a]pyrazin-2-yl)carbamate (D87, 260 mg, 940.9 μmol). LCMS (ES, m/z): 277 [M+H]⁺.

### Synthesis of Intermediate D88

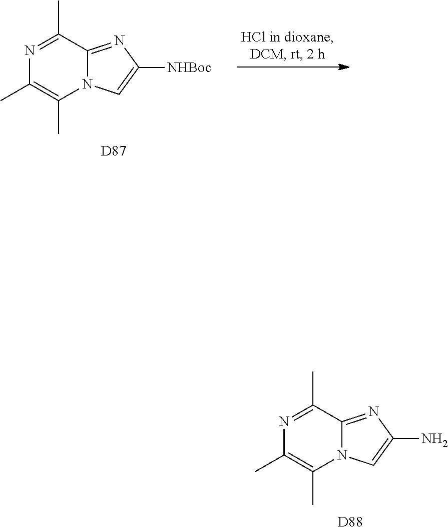

To a stirred mixture of Intermediate D87 (250 mg, 904.7 μmol) in DCM (0.5 mL) was added HCl in dioxane (0.5 mL), and the reaction was stirred for 2 h at room temperature. The resulting mixture was concentrated under reduced pressure, diluted with water (10 mL), basified to pH 8 with saturated NaHCO₃ (aq.), and extracted with DCM: MeOH (10:1, 2×80 mL). The combined organic layers were washed with brine (20 mL), dried over anhydrous Na₂SO₄, and filtered. The filtrate was concentrated under reduced pressure to afford 5,6,8-trimethylimidazo-[1,2-a]-pyrazin-2-amine (D88, 150 mg, 851.2 μmol). LCMS (ES, m/z): 177 [M+H]⁺.

### Example 212: Synthesis of D95

### Synthesis of Intermediate D89

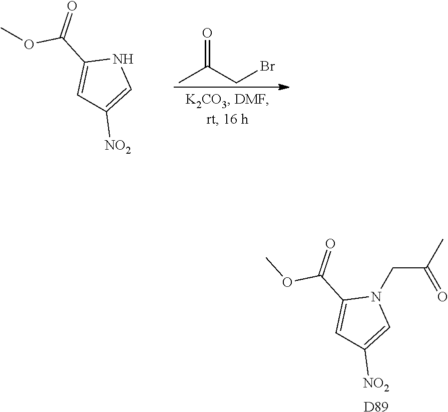

To a stirred mixture of methyl 4-nitro-1H-pyrrole-2-carboxylate (25 g, 147 mmol) in DMF (500 mL) were added K₂CO₃ (40.6 g, 293.9 mmol) and 1-bromopropan-2-one (40.26 g, 293.9 mmol) at room temperature. The resulting mixture was stirred for 16 h, then diluted with water (500 mL), and subsequently extracted with EtOAc (3×500 mL). The combined organic layers were washed with water (3×1 L) and brine (1 μL), dried over anhydrous Na₂SO₄, and filtered. The filtrate was concentrated under reduced pressure and purified by silica gel column chromatography (25% EtOAc in PE) to afford methyl 1-acetonyl-4-nitro-pyrrole-2-carboxylate (D89, 30 g, 132.6 mmol). LCMS (ES, m/z): 227 [M+H]⁺.

### Synthesis of Intermediate D90

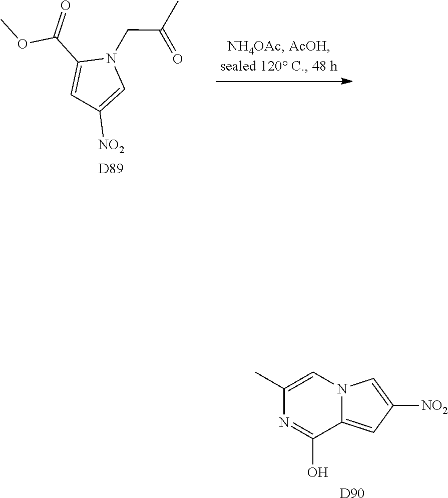

To a stirred mixture of Intermediate D89 (30 g, 132.6 mmol) in AcOH (600 mL) was added NH₄OAc (204.5 g, 2.65 mol), and the reaction was stirred for 48 h at 120° C. The resulting mixture was concentrated under reduced pressure and purified by silica gel column chromatography (50% EtOAc in PE) to afford 3-methyl-7-nitro-pyrrolo[1,2-a]-pyrazin-1-ol (D90, 4.7 g, 24.3 mmol). LCMS (ES, m/z): 192 [M−H]⁻.

### Synthesis of Intermediate D91

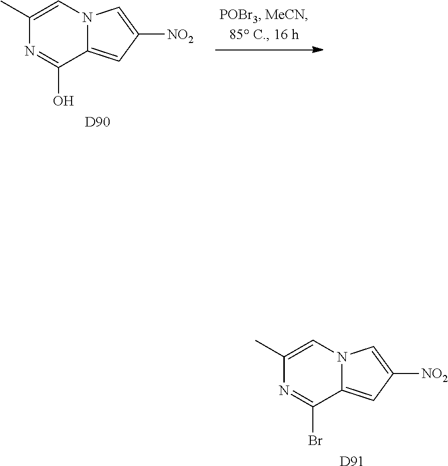

To a stirred mixture of Intermediate D90 (4.2 g, 21.7 mmol) in MeCN (84 mL) was added POBr₃ (18.7 g, 65.2 mmol) at room temperature. The resulting mixture was stirred for 16 h at 85° C., then quenched with water at 0° C., subsequently further diluted with water (80 mL), and later extracted with EtOAc (3×80 mL). The combined organic layers were washed with NaHCO₃ (3×150 mL) and water (150 mL), dried over anhydrous Na₂SO₄, and filtered. The filtrate was concentrated under reduced pressure and purified by silica gel column chromatography (33% EtOAc in PE) to afford 1-bromo-3-methyl-7-nitro-pyrrolo[1,2-a]pyrazine (D91, 1.9 g, 7.4 mmol). LCMS (ES, m/z): 297 [M+H+CH₃CN]⁺.

### Synthesis of Intermediate D92

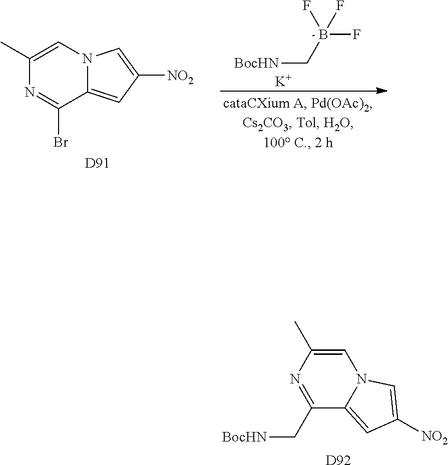

To a stirred mixture of Intermediate D91 (1.8 g, 7.0 mmol) and potassium (((tert-butoxycarbonyl)amino)methyl)trifluoroborate (3.33 g, 14.1 mmol) in toluene (25 mL) were added H₂O (1 mL), Cs₂CO₃ (6.87 g, 21.1 mmol), Pd(OAc)₂ (157.8 mg, 703 μmol) and cataCXium A (504.7 mg, 1.4 mmol), and the reaction was stirred for 2 h at 100° C. under N₂. The resulting mixture was concentrated under reduced pressure and purified by silica gel column chromatography (100% EtOAc) to afford tert-butyl N-[(3-methyl-7-nitro-pyrrolo[1,2-a]pyrazin-1-yl)methyl]carbamate (D92, 900 mg, 2.94 mmol). LCMS (ES, m/z): 307 [M+H]⁺.

### Synthesis of Intermediate D93

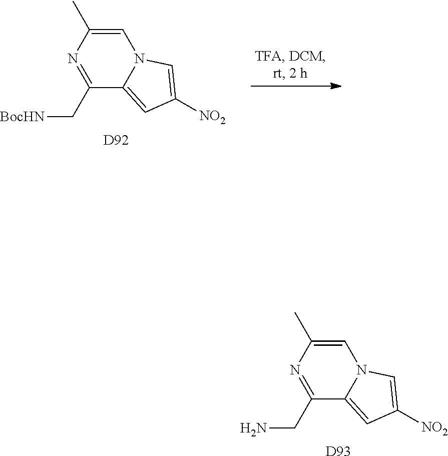

To a stirred mixture of Intermediate D92 (600 mg, 1.96 mmol) in DCM (4 mL) was added TFA (3 mL) dropwise, and the reaction was stirred for 2 h at room temperature. The resulting mixture was concentrated under reduced pressure to afford (3-methyl-7-nitropyrrolo[1,2-a]pyrazin-1-yl) methanamine 2,2,2-trifluoroacetate (D93, 750 mg, crude), which was used directly in the next step. LCMS (ES, m/z): 207 [M+H]⁺.

### Synthesis of Intermediate D94

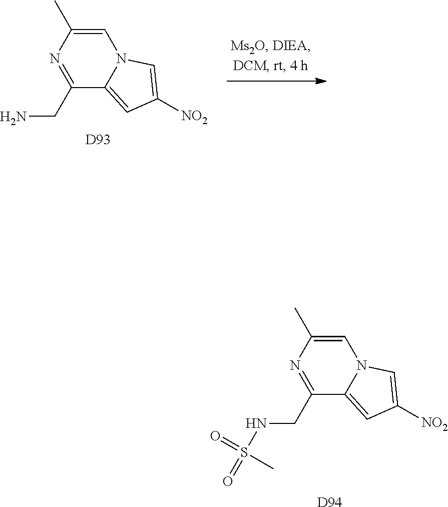

Intermediate D93 (crude, without further purification) was diluted with DCM (15 mL). After cooling the mixture to 5° C., DIEA (758.8 mg, 5.9 mmol) was added, and, after stirring for a further 5 min, another portion of Ms₂O (341.2 mg, 1.8 mmol) was added. The resulting mixture was stirred for 4 h at room temperature, then quenched with water (20 mL), and subsequently extracted with DCM (3×15 mL). The combined organic layers were washed with water (3×40 mL) and brine (40 mL), dried over anhydrous Na₂SO₄, and filtered. The filtrate was concentrated under reduced pressure and purified by silica gel column chromatography (9% MeOH in DCM) to afford N-[(3-methyl-7-nitro-pyrrolo[1,2-a]pyrazin-1-yl)methyl]-methanesulfonamide (D94, 120 mg, 422.1 μmol). LCMS (ES, m/z): 285 [M+H]⁺.

### Synthesis of Intermediate D95

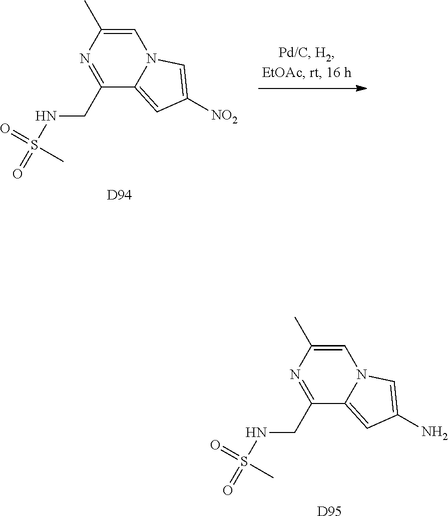

To a solution of Intermediate D94 (120 mg, 422.1 μmol) in EtOAc (3 mL) was added Pd/C (40 mg) under N₂, and the reaction was hydrogenated at room temperature for 16 h under hydrogen atmosphere using a hydrogen balloon. The resulting mixture was filtered through a Celite pad and concentrated under reduced pressure to afford N-[(7-amino-3-methyl-pyrrolo[1,2-a]pyrazin-1-yl)methyl]methanesulfonamide (D95, 90 mg, 353.9 μmol). LCMS (ES, m/z): 255 [M+H]⁺.

### Example 213: Synthesis of D100

### Synthesis of Intermediate D96

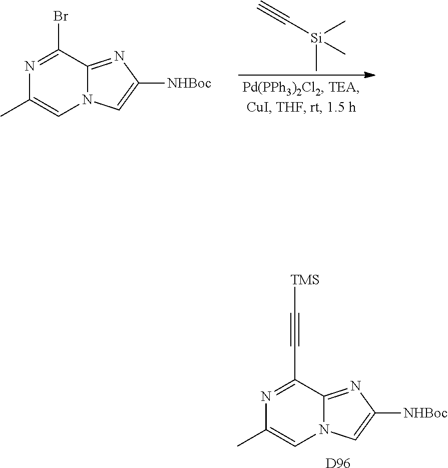

To a stirred mixture of tert-butyl N-(8-bromo-6-methyl-imidazo[1,2-a]pyrazin-2-yl)carbamate (1.0 g, 3.1 mmol) and ethynyl(trimethyl)silane (450.3 mg, 4.6 mmol) in THF (20 mL) were added TEA (1.5 g, 15.3 mmol), CuI (58.2 mg, 305.7 μmol), and Pd(PPh₃)₂Cl₂ (213.9 mg, 305.7 μmol) at room temperature under N₂. The resulting mixture was stirred for 1.5 h, then allowed to cool to room temperature, diluted with water (100 mL), and subsequently extracted with EtOAc (2×100 mL). The combined organic layers were washed with brine (100 mL), dried over anhydrous Na₂SO₄, and filtered. The filtrate was concentrated under reduced pressure and purified by silica gel column chromatography (25% EtOAc in PE) to afford tert-butyl N-[6-methyl-8-(2-trimethylsilylethynyl)imidazo[1,2-a]pyrazin-2-yl]carbamate (D96, 800 mg, 2.32 mmol). LCMS (ES, m/z): 345 [M+H]⁺.

### Synthesis of Intermediate D97

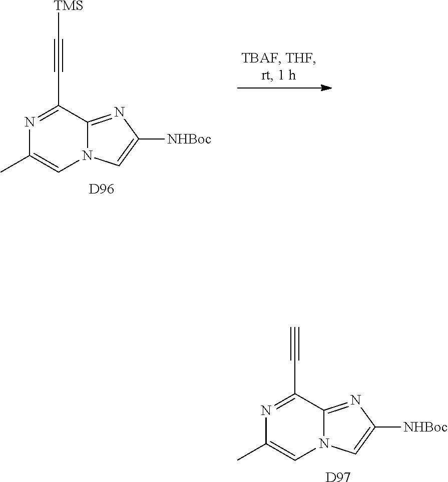

To a stirred mixture of Intermediate D96 (780.0 mg, 2.3 mmol) in THF (15 mL) was added TBAF (949.2 mg, 3.4 mmol), and the reaction was stirred for 1 h at room temperature. The resulting mixture was quenched with water (50 mL) and extracted with EtOAc (2×50 mL). The combined organic layers were washed with brine (3×50 mL), dried over anhydrous Na₂SO₄, filtered, and the filtrate was concentrated under reduced pressure to afford tert-butyl N-(8-ethynyl-6-methyl-imidazo[1,2-a]pyrazin-2-yl)carbamate (D97, 380 mg, 1.4 mmol). LCMS (ES, m/z): 273 [M+H]⁺.

### Synthesis of Intermediate D98

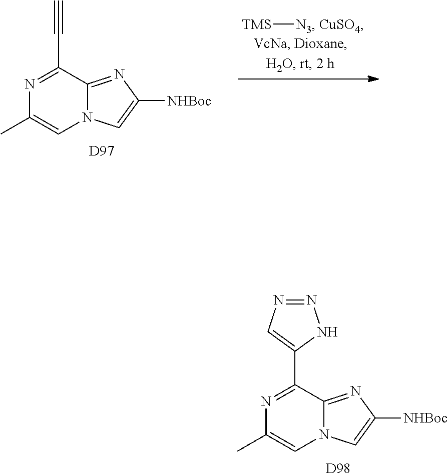

To a stirred mixture of Intermediate D97 (280.0 mg, 1.0 mmol) in dioxane (4 mL) and H₂O (0.5 mL) were added TMS-N₃ (236.5 mg, 2.1 mmol), CuSO₄ (16.4 mg, 102.8 μmol, 4.6 μL), and VcNa (40.7 mg, 205.7 μmol) at room temperature under N₂. The resulting mixture was stirred for 2 h, then quenched with water (50 mL) at room temperature, and subsequently extracted with EtOAc (2×50 mL). The combined organic layers were washed with brine (50 mL), dried over anhydrous Na₂SO₄, and filtered. The filtrate was concentrated under reduced pressure and purified by silica gel column chromatography (25% EtOAc in PE) to afford tert-butyl N-[6-methyl-8-(1H-triazol-5-yl)imidazo[1,2-a]pyrazin-2-yl]carbamate (D98, 140 mg, 444 μmol). LCMS (ES, m/z): 316 [M+H]⁺.

### Synthesis of Intermediate D99

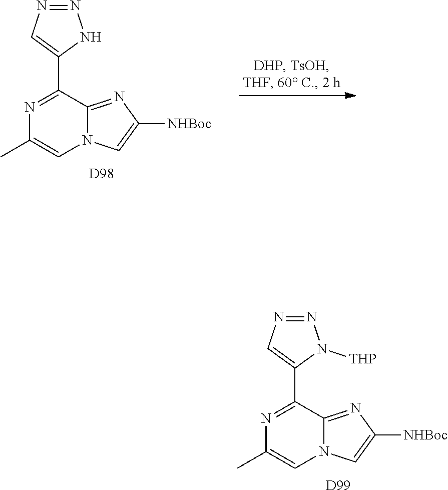

To a stirred mixture of Intermediate D98 (130.0 mg, 412.3 μmol) in THF (3 mL) were added DHP (22.2 mg, 618.4 μmol) and TsOH (7.1 mg, 41.2 μmol), and the reaction was stirred for 2 h at 60° C. under N₂. The resulting mixture was quenched with water (10 ml) at 25° C. and extracted with EtOAc (2×10 mL). The combined organic layers were washed with brine (10 mL), dried over anhydrous Na₂SO₄, and filtered. The filtrate was concentrated under reduced pressure and purified by silica gel column chromatography (33% EtOAc in PE) to afford tert-butyl N-[6-methyl-8-(1-tetrahydropyran-2-yltriazol-4-yl)imidazo[1,2-a]pyrazin-2-yl]carbamate (D99, 55 mg, 137.7 μmol). LCMS (ES, m/z): 400 [M+H]⁻.

### Synthesis of Intermediate D100

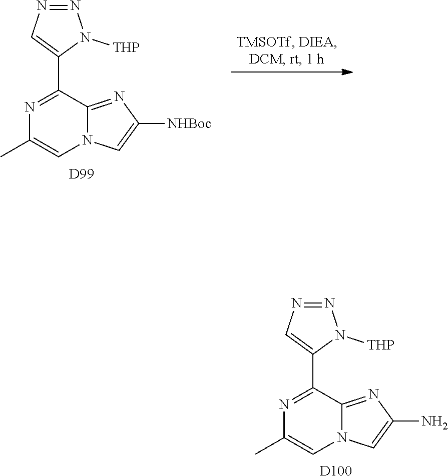

To a solution of Intermediate D99 (50.0 mg, 125.2 μmol) in DCM (1 mL) were added TMSOTf (83.4 mg, 375.5 μmol) and DIEA (32.3 mg, 250.4 μmol). The reaction was stirred for 1 h at room temperature, then concentrated under reduced pressure and adjusted to pH 8 with saturated NaHCO₃ (aq.), further diluted with water (5 mL) and subsequently extracted with DCM/MeOH (20/1) (2×10 mL). The combined organic layers were washed with water (20 mL), brine (20 mL), dried over anhydrous Na₂SO₄, filtered, and the filtrate was concentrated under reduced pressure to afford 6-methyl-8-(1-tetrahydropyran-2-yltriazol-4-yl)imidazo[1,2-a]pyrazin-2-amine (D100, 35 mg, 116.9 μmol). LCMS (ES, m/z): 300 [M+H]⁺.

### Example 214: Synthesis of D103

### Synthesis of D101

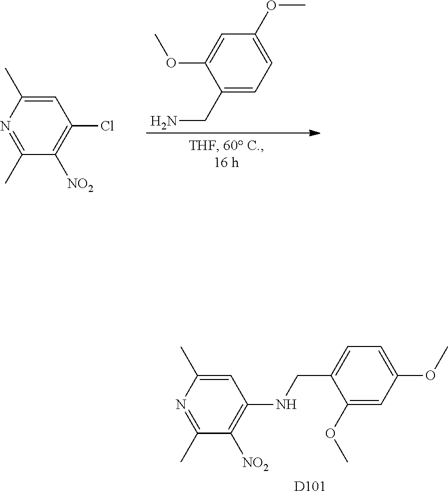

To a stirred mixture of 4-chloro-2,6-dimethyl-3-nitro-pyridine (2 g, 10.7 mmol) and (2,4-dimethoxyphenyl) methanamine (3.6 g, 21.44 mmol) in THF (40 mL), and the reaction was stirred for 16 h at 60° C. The resulting mixture was diluted with water (40 mL) and extracted with EtOAc (3×40 mL). The combined organic layers were washed with water (3×100 mL) and brine (100 mL), dried over anhydrous Na₂SO₄, and filtered. The filtrate was concentrated under reduced pressure and purified by silica gel column chromatography (50% EtOAc in PE) to afford N-[(2,4-dimethoxyphenyl)methyl]-2,6-dimethyl-3-nitro-pyridin-4-amine (D101, 3 g, 9.45 mmol, 88% yield) as an oil. LCMS (ES, m/z): 318 [M+H]⁺.

### Synthesis of D102

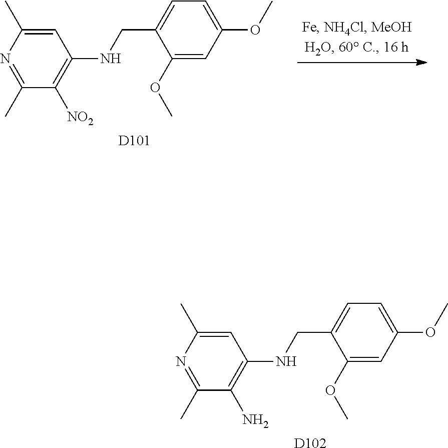

To a stirred mixture of N-[(2,4-dimethoxyphenyl)methyl]-2,6-dimethyl-3-nitro-pyridin-4-amine (D101, 3 g, 9.5 mmol) in MeOH (15 mL) were added H₂O (15 mL) and NH₄Cl (5.06 g, 94.5 mmol) and Fe (2.64 g, 47.3 mmol), and the reaction was stirred for 16 h at 60° C. under N₂. The resulting mixture was filtered, the filter cake was washed with EtOAc (3×30 mL). The filtrate was extracted with EtOAc (3×50 mL). The combined organic layers were washed with water (3×100 mL) and brine (100 mL), dried over anhydrous Na₂SO₄, and filtered. The filtrate was concentrated under reduced pressure and purified by silica gel column chromatography (9% MeOH in DCM) to afford N4-[(2,4-dimethoxyphenyl)methyl]-2,6-dimethyl-pyridine-3,4-diamine (D102, 2.1 g, 7.31 mmol, 77% yield) as a solid. LCMS (ES, m/z): 288 [M+H]⁺.

### Synthesis of D103

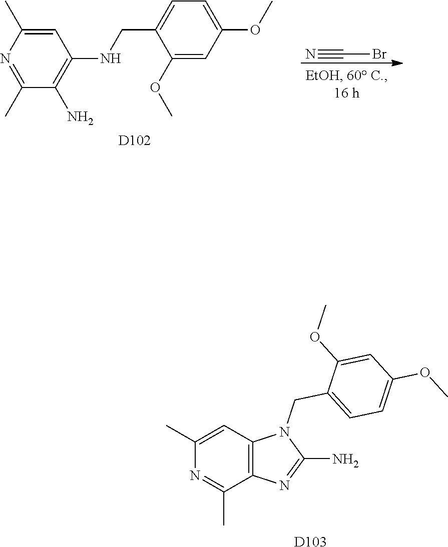

To a stirred mixture of N4-[(2,4-dimethoxyphenyl)methyl]-2,6-dimethyl-pyridine-3,4-diamine (D102, 500 mg, 1.74 mmol) in EtOH (10 mL) was added carbononitridic bromide (239.6 mg, 2.26 mmol), and the reaction was stirred for 16 h at 60° C. The resulting mixture was diluted with water (10 mL) and extracted with EtOAc (3×10 mL). The combined organic layers were washed with water (3×30 mL) and brine (30 mL), dried over anhydrous Na₂SO₄, and filtered. The filtrate was concentrated under reduced pressure and purified by silica gel column chromatography (100% EtOAc) to afford 1-[(2,4-dimethoxyphenyl)methyl]-4,6-dimethyl-imidazo[4,5-c]pyridin-2-amine (D103, 50 mg, 160.1 μmol, 9% yield) as a solid. LCMS (ES, m/z): 313 [M+H]⁺.

### Example 215: Synthesis of Compound 582

### Synthesis of Intermediate D104

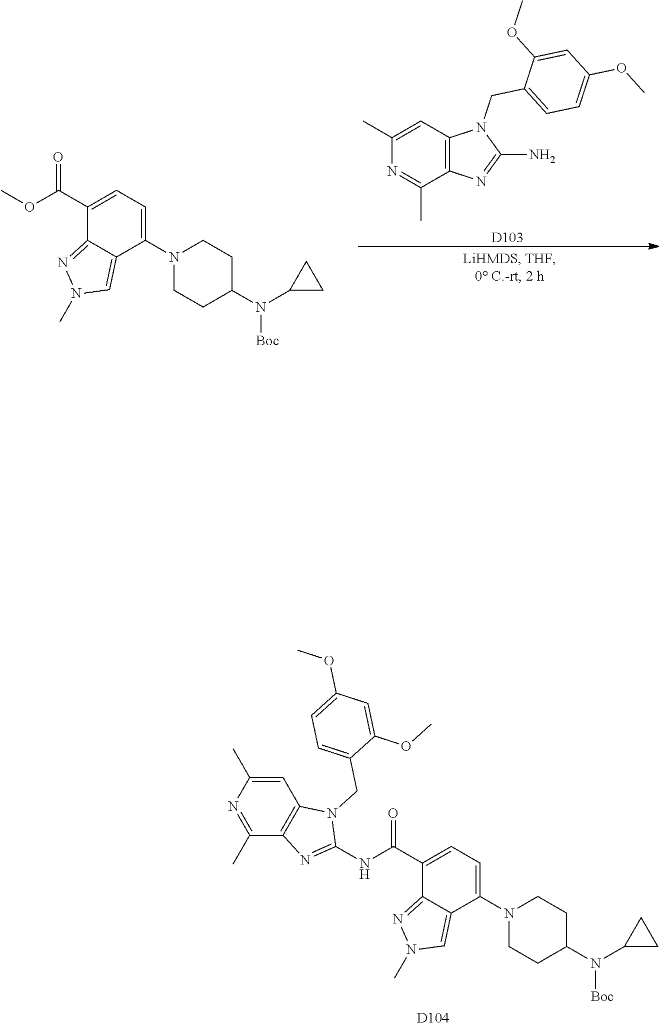

To a stirred mixture of methyl 4-[4-[tert-butoxycarbonyl(cyclopropyl)amino]-1-piperidyl]-2-methyl-indazole-7-carboxylate (60 mg, 140 μmol) and 1-[(2,4-dimethoxyphenyl)methyl]-4,6-dimethyl-imidazo[4,5-c]pyridin-2-amine (43.7 mg, 140 μmol) in THF (1.5 mL) was added LiHMDS (420 μL, 420 μmol) dropwise at room temperature under N₂. The resulting mixture was stirred for 2 h, then quenched with water at 0° C., and subsequently extracted with EtOAc (3×5 mL). The combined organic layers were washed with water (3×15 mL) and brine (15 mL), dried over anhydrous Na₂SO₄, and filtered. The filtrate was concentrated under reduced pressure and purified by silica gel column chromatography (100% EtOAc) to afford tert-butyl N-cyclopropyl-N-[1-[7-[[1-[(2,4-dimethoxyphenyl)methyl]-4,6-dimethyl-imidazo[4,5-c]pyridin-2-yl]carbamoyl]-2-methyl-indazol-4-yl]-4-piperidyl]carbamate (D104, 50 mg, 70.5 μmol). LCMS (ES, m/z): 709 [M+H]⁺.

### Synthesis of Compound 582

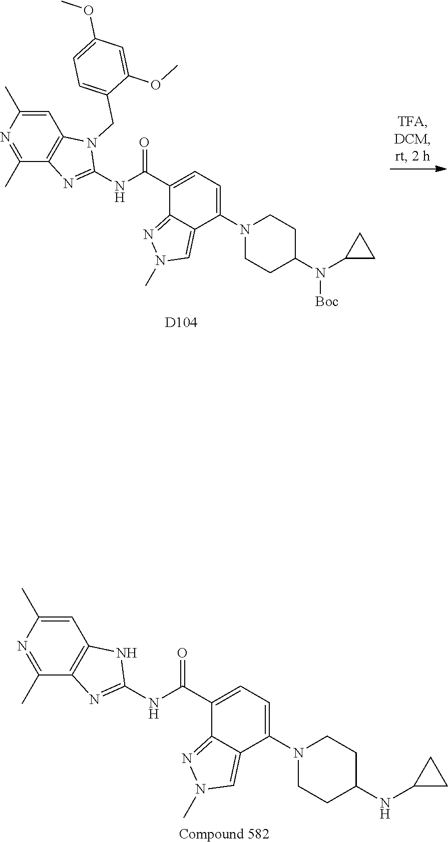

To a stirred mixture of Intermediate D104 (50 mg, 70.5 μmol) in DCM (1 mL) was added TFA (0.25 mL) dropwise, and the reaction was stirred for 2 h at room temperature. The resulting mixture was concentrated under reduced pressure and purified by Prep-HPLC (Condition 5, Gradient 30) to afford 4-[4-(cyclopropylamino)-1-piperidyl]-N-(4,6-dimethyl-1H-imidazo[4,5-c]pyridin-2-yl)-2-methyl-indazole-7-carboxamide (Compound 582, 6.9 mg, 15.1 μmol). LCMS (ES, m/z): 459 [M+H]⁺. ¹H NMR (400 MHz, DMSO-d₆) δ 12.33 (s, 1H), 11.88 (s, 1H), 8.86 (s, 1H), 8.08 (d, J=8.3 Hz, 1H), 7.15 (s, 1H), 6.54 (d, J=8.4 Hz, 1H), 4.30 (s, 3H), 3.96 (d, J=12.8 Hz, 2H), 3.16 (t, J=11.6 Hz, 2H), 2.82 (s, 1H), 2.62 (s, 3H), 2.47 (s, 3H), 2.28 (s, 1H), 2.13 (tt, J=6.7, 3.6 Hz, 1H), 2.01 (d, J=12.2 Hz, 2H), 1.47 (q, J=9.8 Hz, 2H), 0.40 (td, J=6.3, 4.0 Hz, 2H), 0.28-0.20 (m, 2H).

An analogous method was followed to obtain the following compounds.

| | Starting | |
|---|---|---|
| Compound | Material | Characterization |
| 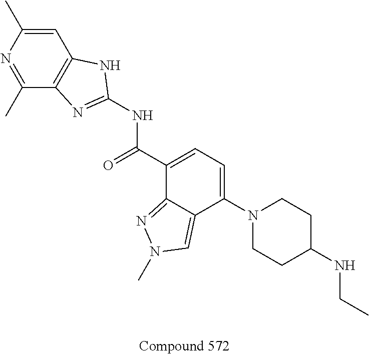 | 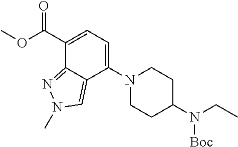 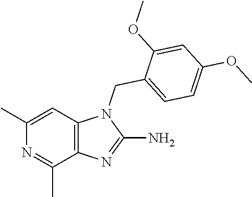 | The crude product was purified by Prep-HPLC (Condition 14, Gradient 6) to afford N-(4,6- dimethyl-1H-imidazo[4,5-c]- pyridin-2-yl)-4-[4-(ethylamino)- 1-piperidyl]-2-methyl-indazole- 7-carboxamide (Compound 572, 15 mg, 33.6 μmol, 39% yield) as a solid. LCMS (ES, m/z): 547 [M + H]⁺. ¹H NMR (400 MHz, DMSO-d₆) δ 11.88 (s, 1H), 8.85 (s, 1H), 8.08 (d, J = 8.2 Hz, 1H), 7.15 (s, 1H), 6.54 (d, J = 8.4 Hz, 1H), 4.30 (s, 3H), 3.96 (d, J = 12.9 Hz, 2H), 3.15 (t, J = 11.7 Hz, 2H), 2.71 (dt, J = 10.0, 5.6 Hz, 1H), 2.66- 2.56 (m, 5H), 2.47 (s, 3H), 1.97 (d, J = 12.8 Hz, 2H), 1.50-1.36 (m, 2H), 1.04 (t, J = 7.1 Hz, 3H). |
| 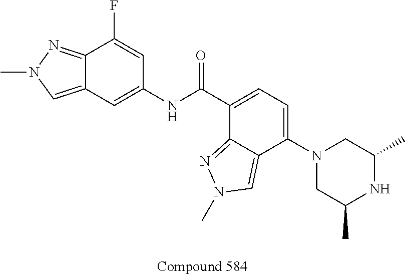 | 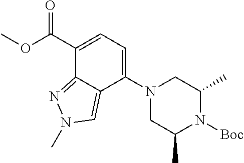 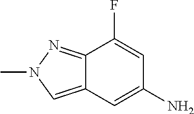 | Modifications: Step 1 was ran at room temperature for 1 h. The crude product was purified by Prep-HPLC (Condition 6, Gradient 10) to afford 4-[(3S, 5S)-3,5-dimethylpiperazin-1- yl]-N-(7-fluoro-2-methyl- indazol-5-yl)-2-methyl- indazole-7-carboxamide (Compound 584, 16.9 mg, 38.8 μmol). LCMS (ES, m/z): 436 [M + H]⁺. ¹H NMR (400 MHz, DMSO-d₆) δ 11.15 (s, 1H), 8.65 (s, 1H), 8.40 (d, J = 2.8 Hz, 1H), 8.03 (d, J = 1.6 Hz, 1H), 7.95 (d, J = 8.1 Hz, 1H), 7.41 (dd, J = 13.2, 1.6 Hz, 1H), 6.44 (d, J = 8.2 Hz, 1H), 4.29 (s, 3H), 4.17 (s, 3H), 3.37 (dd, J = 11.8, 3.3 Hz, 2H), 3.25 (td, J = |
| | | 6.4, 3.2 Hz, 2H), 3.07 (dd, J = |
| | | 11.8, 6.4 Hz, 2H), 2.16-2.15 (s, |
| | | 1H), 1.14 (d, J = 6.4 Hz, 6H). |
| 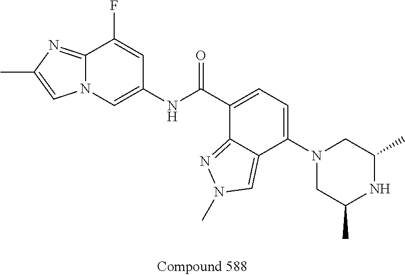 | 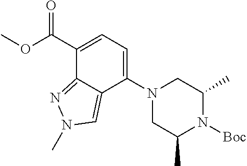 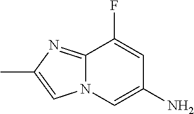 | Modification: Step 1 was ran for 1 h at room temperature. The crude product was purified by Prep-HPLC (Condition 6, Gradient 12) to afford 4-[(3S, 5S)-3,5-dimethylpiperazin-1- yl]-N-(8-fluoro-2-methyl- imidazo[1,2-a]pyridin-6-yl)- 2-methyl-indazole-7- carboxamide (Compound 588, 26.2 mg, 60.2 μmol). LCMS (ES, m/z): 436 [M + H]⁺. ¹H NMR (400 MHz, DMSO-d₆) δ 11.33 (s, 1H), 9.60 (d, J = 14.4 Hz, 3H), 8.83 (s, 1H), 8.26 (s, 1H), 8.04 (dd, J = 16.1, 9.8 Hz, 2H), 6.62 (d, J = 8.1 Hz, 1H), 4.34 (s, 3H), 3.70 (dd, J = 13.3, 3.3 Hz, 2H), 3.44 (dd, J = 13.3, 6.5 Hz, 2H), 2.49 (s, 3H), 1.44 |
| | | (d, J = 6.6 Hz, 6H). |
| 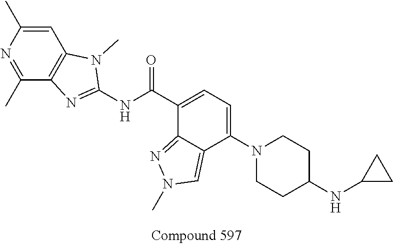 [SMILES: Cc1cc2c(nc(NC(=O)c3ccc(N4CCC(NC5CC5)CC4)c4cn(C)nc34)n2C)c(C)n1] | 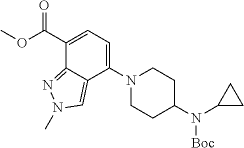 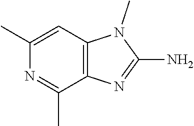 | Modification: Step 1 was ran at room temperature for 2 h. Step 2 was ran using HCl in dioxane and DCM at room temperature for 2 h. The crude product was purified by Prep-HPLC (Condition 5, Gradient 34) to afford 4-[4-(cyclopropylamino)- 1-piperidyl]-2-methyl-N-(1,4,6- trimethylimidazo[4,5-c]- pyridin-2-yl)indazole-7- carboxamide (Compound 597, 40 mg, 84.6 μmol). LCMS (ES, m/z): 473 [M + H]⁺. ¹H NMR (400 MHz, DMSO-d₆) δ 11.21 (s, 1H), 8.80 (s, 1H), 7.98 (d, J = 8.1 Hz, 1H), 7.24 (s, 1H), 6.49 (d, J = 8.3 Hz, 1H), 4.23 (s, 3H), 3.91 (d, J = 12.9 |
| | | Hz, 2H), 3.65 (s, 3H), 3.10 (t, |
| | | J = 11.7 Hz, 2H), 2.78 (dt, J = |
| | | 9.8, 5.5 Hz, 1H), 2.61 (s, 3H), |
| | | 2.51 (s, 3H), 2.25 (s, 1H), 2.10 |
| | | (tt, J = 6.7, 3.6 Hz, 1H), 1.99 (d, |
| | | J = 12.6 Hz, 2H), 1.45 (d, J = |
| | | 11.1 Hz, 2H), 0.38 (td, J = 6.3, |
| | | 4.0 Hz, 2H), 0.25-0.17 (m, 2H). |

### Example 216: Synthesis of Compound 522

### Synthesis of Intermediate D105

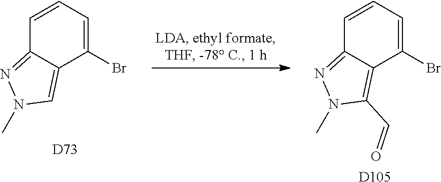

A solution of Intermediate D73 (8.0 g, 37.9 mmol) in THF (10 mL) was added dropwise LDA (2.0 M in THF) (38 mL, 56.9 mmol) at −78° C. under N₂ atmosphere. The reaction mixture was stirred at −78° C. for 1 h. Then a solution of ethyl formate (5.62 g, 75.8 mmol) in 20 mL of THE was added dropwise and the mixture was stirred for another 30 min at −78° C. The reaction was quenched with water/sat. NH₄Cl (200 mL), and later extracted with EtOAc (2×200 mL). The combined organic extracts were washed with brine (400 mL), dried over anhydrous Na₂SO₄, and filtered. The filtrate was concentrated under reduced pressure and purified by silica gel column chromatography (25% EtOAc in PE) to afford 4-bromo-2-methyl-indazole-3-carbaldehyde (D105, 8 g, 33.5 mmol). LCMS (ES, m/z): 239 [M+H]⁺.

### Synthesis of Intermediate D106

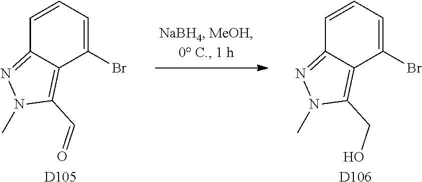

To a stirred mixture of Intermediate D105 (8.0 g, 33.5 mmol) in MeOH (160 mL) was added NaBH₄ (1.9 g, 50.2 mmol) in portions at 0° C. under N₂. The resulting mixture was stirred for 1 h at 0° C. under N₂, then quenched with water (400 mL) at 0° C. and subsequently extracted with EtOAc (2×400 mL). The combined organic layers were washed with brine (400 mL), dried over anhydrous Na₂SO₄, and filtered. The filtrate was concentrated under reduced pressure and purified by silica gel column chromatography (50% EtOAc in PE) to afford (4-bromo-2-methyl-indazol-3-yl)-methanol (D106, 6 g, 24.9 mmol). LCMS (ES, m/z): 241 [M+H]⁺.

### Synthesis of Intermediate D107

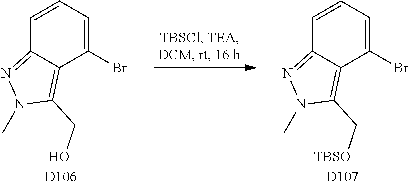

To a solution of Intermediate D106 (1.5 g, 6.2 mmol) in DCM (15 mL) was added DIEA (2.4 g, 18.7 mmol, 3.25 mL) and TBSCl (1.9 g, 12.4 mmol), and the reaction was stirred for 16 h at rt. The resulting mixture was diluted with water (50 mL) and extracted with DCM (2×50 mL). The combined organic layers were washed with brine (100 mL), dried over anhydrous Na₂SO₄, and filtered. The filtrate was concentrated under reduced pressure and purified by silica gel column chromatography (25% EtOAc in PE) to afford (4-bromo-2-methyl-indazol-3-yl)methoxy-tert-butyl-dimethyl-silane (D107, 1.6 g, 4.5 mmol). LCMS (ES, m/z): 355 [M+H]⁺.

### Synthesis of Intermediate D108

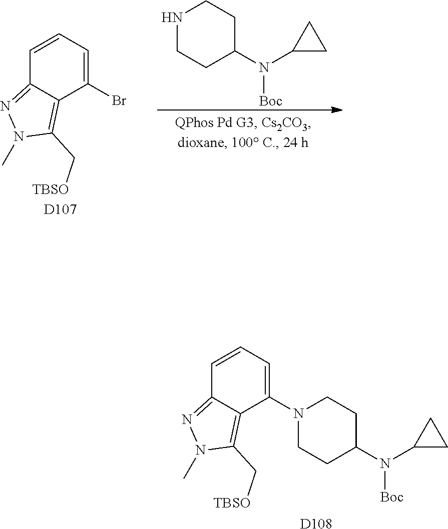

To a solution of Intermediate D107 (0.83 g, 2.3 mmol) and tert-butyl N-cyclopropyl-N-(4-piperidyl)carbamate (561.3 mg, 2.3 mmol) in dioxane (8 mL) were added Cs₂CO₃ (1.52 g, 4.7 mmol) and QPhos Pd G3 (251.4 mg, 233.6 μmol), and the reaction was stirred for 24 h at 100° C. under N₂. The resulting mixture was concentrated under reduced pressure and purified by silica gel column chromatography (50% EtOAc in PE) to afford tert-butyl N-[1-[3-[[tert-butyl(dimethyl)silyl]oxymethyl]-2-methyl-indazol-4-yl]-4-piperidyl]-N-cyclopropyl-carbamate (D108, 0.55 g, 1.07 mmol). LCMS (ES, m/z): 515 [M+H]⁺.

### Synthesis of Intermediate D109

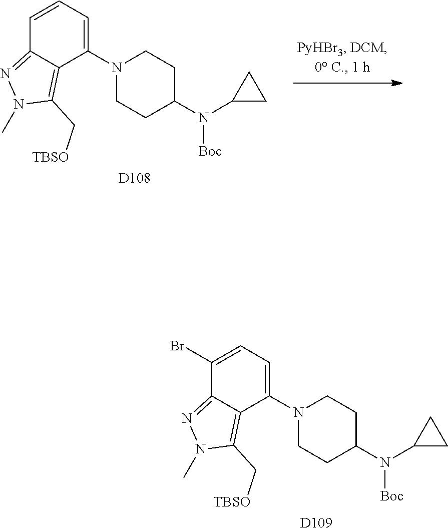 [SMILES: Cn1nc2cccc(N3CCC(N(C(=O)OC(C)(C)C)C4CC4)CC3)c2c1CO[Si](C)(C)C(C)(C)C ; "D108" ; Cn1nc2c(Br)ccc(N3CCC(N(C(=O)OC(C)(C)C)C4CC4)CC3)c2c1CO[Si](C)(C)C(C)(C)C ; "D109"]

To a stirred mixture of Intermediate D108 (0.5 g, 971.3 μmol) in DCM (5 mL) was added PyHBr₃ (217.4 mg, 679.9 μmol) in portions at 0° C. The resulting mixture was stirred for 1 h at 0° C., then quenched with water (10 mL) at 0° C., and extracted with EtOAc (2×50 mL). The combined organic layers were washed with brine (50 mL), dried over anhydrous Na₂SO₄, and filtered. The filtrate was concentrated under reduced pressure and purified by silica gel column chromatography (25% EtOAc in PE) to afford tert-butyl N-[1-[7-bromo-3-[[tert-butyl(dimethyl) silyl]oxymethyl]-2-methyl-indazol-4-yl]-4-piperidyl]-N-cyclopropyl-carbamate (D109, 0.35 g, 589.6 μmol). LCMS (ES, m/z): 593 [M+H]⁻.

### Synthesis of Intermediate D110

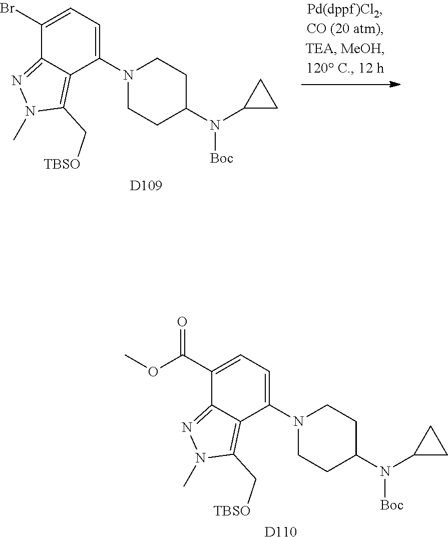

To a solution of Intermediate D109 (320 mg, 539 μmol) in MeOH (5 mL) was added TEA (218.1 mg, 2.16 mmol) and Pd(dppf)Cl₂ (157.7 mg, 215.6 μmol) in a pressure tank. The mixture was purged with nitrogen for 12 h and then pressurized to 2 MPa with carbon monoxide at 120° C. for 12 h. The reaction mixture was cooled to room temperature, concentrated under reduced pressure, and purified by silica gel column chromatography (67% EtOAc in PE) to afford methyl 4-[4-[tert-butoxycarbonyl(cyclopropyl)amino]-1-piperidyl]-3-[[tert-butyl(dimethyl)silyl]oxymethyl]-2-methyl-indazole-7-carboxylate (D110, 300 mg, 523.7 μmol). LCMS (ES, m/z): 573 [M+H]⁺.

### Synthesis of Intermediate D111

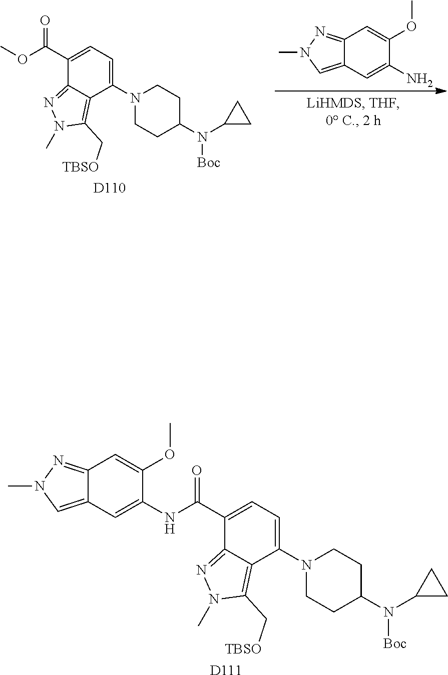

To a stirred mixture of Intermediate D110 (0.1 g, 174.6 μmol) and 6-methoxy-2-methyl-indazol-5-amine (30.9 mg, 174.6 μmol) in THF (1 mL) was added LiHMDS (146.0 mg, 872.9 μmol) in portions at 0° C., and the reaction was stirred for 2 h at 0° C. under N₂. The resulting mixture was quenched with water (10 mL) and extracted with EtOAc (2×50 mL). The combined organic layers were washed with brine (50 mL), dried over anhydrous Na₂SO₄, and filtered. The filtrate was concentrated under reduced pressure and purified by silica gel column chromatography (50% EtOAc in PE) to afford tert-butyl N-[1-[3-[[tert-butyl(dimethyl)silyl]-oxymethyl]-7-[(6-methoxy-2-methyl-indazol-5-yl)carbamoyl]-2-methyl-indazol-4-yl]-4-piperidyl]-N-cyclopropyl-carbamate (D111, 70 mg, 97.5 μmol). LCMS (ES, m/z): 718 [M+H]⁺.

### Synthesis of Compound 522

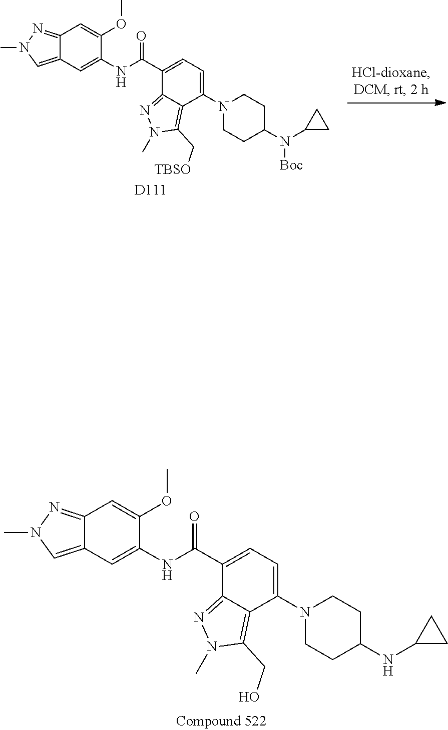 [SMILES: COc1cc2nn(C)cc2cc1NC(=O)c1ccc(N2CCC(N(C(=O)OC(C)(C)C)C3CC3)CC2)c2c(CO[Si](C)(C)C(C)(C)C)n(C)nc12 ; "D111" ; COc1cc2nn(C)cc2cc1NC(=O)c1ccc(N2CCC(NC3CC3)CC2)c2c(CO)n(C)nc12]

To a solution of Intermediate D111 (100.0 mg, 139.3 μmol) in DCM (2 mL) was added HCl-dioxane (1 mL). The reaction was stirred for 2 h at room temperature, then concentrated under reduced pressure and adjusted to pH 8 with saturated NaHCO₃ (aq.). The resulting mixture was diluted with water (5 mL) and extracted with DCM/MeOH (20/1) (2×10 mL). The combined organic layers were washed with water (20 mL), brine (20 mL), dried over anhydrous Na₂SO₄, and filtered. The filtrate was concentrated under reduced pressure and purified by reversed-phase flash chromatography (Condition 3, Gradient 20) to afford 4-[4-(cyclopropylamino)-1-piperidyl]-3-(hydroxymethyl)-N-(6-methoxy-2-methyl-indazol-5-yl)-2-methyl-indazole-7-carboxamide (Compound 522, 4.4 mg, 8.6 μmol). LCMS (ES, m/z): 504 [M+H]⁺. ¹H NMR (400 MHz, DMSO-d₆) δ 11.67 (s, 1H), 8.85 (s, 1H), 8.20 (s, 1H), 8.02 (d, J=7.9 Hz, 1H), 7.09 (s, 1H), 6.69 (d, J=7.9 Hz, 1H), 5.58 (t, J=4.7 Hz, 1H), 5.05 (d, J=4.8 Hz, 2H), 4.31 (s, 3H), 4.08 (s, 3H), 4.05 (s, 3H), 3.49 (d, J=11.7 Hz, 2H), 3.38-3.34 (m, 1H), 2.83-2.70 (m, 3H), 2.28-2.25 (m, 1H), 2.13 (dd, J=6.7, 3.3 Hz, 1H), 2.03 (d, J=12.5 Hz, 2H), 1.56 (t, J=11.5 Hz, 2H), 0.40 (dt, J=6.1, 3.0 Hz, 2H), 0.25 (p, J=3.8 Hz, 2H).

### Example 217: Synthesis of Compound 600

### Synthesis of Intermediate D112

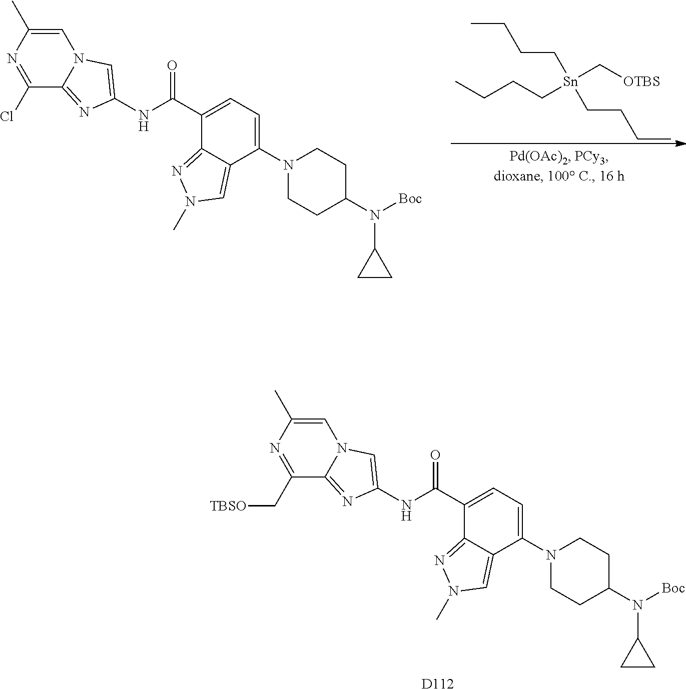 [SMILES: Cc1cn2cc(NC(=O)c3ccc(N4CCC(N(C(=O)OC(C)(C)C)C5CC5)CC4)c4cn(C)nc34)nc2c(Cl)n1 ; CCC[CH2][Sn]([CH2]CCC)([CH2]CCC)[CH2]O[Si](C)(C)C(C)(C)C ; Cc1cn2cc(NC(=O)c3ccc(N4CCC(N(C(=O)OC(C)(C)C)C5CC5)CC4)c4cn(C)nc34)nc2c(CO[Si](C)(C)C(C)(C)C)n1 ; "D112"]

To a stirred mixture of tert-butyl N-[1-[7-[(8-chloro-6-methyl-imidazo[1,2-a]pyrazin-2-yl)carbamoyl]-2-methyl-indazol-4-yl]-4-piperidyl]-N-cyclopropyl-carbamate (400.0 mg, 690.7 μmol) and tert-butyl-dimethyl-(tributylstannylmethoxy)silane (300.7 mg, 690.7 μmol) in dioxane (8 mL) were added PCy₃ (77.4 mg, 276.3 μmol), and Pd(OAc)₂ (31.0 mg, 138.2 μmol) at room temperature under N₂. The resulting mixture was stirred for 16 h at 100° C. under N₂, then allowed to cool to room temperature, diluted with water (10 mL), and subsequently extracted with EtOAc (2×20 mL). The combined organic layers were washed with water (2×40 mL), brine (40 mL), dried over anhydrous Na₂SO₄, and filtered. The filtrate was concentrated under reduced pressure and purified by silica gel column chromatography (75% EtOAc in PE) to afford tert-butyl N-[1-[7-[[8-[[tert-butyl(dimethyl)silyl]oxymethyl]-6-methyl-imidazo[1,2-a]-pyrazin-2-yl]carbamoyl]-2-methyl-indazol-4-yl]-4-piperidyl]-N-cyclopropyl-carbamate (D112, 190 mg, 275.8 μmol). LCMS (ES, m/z): 689 [M+H]⁺.

### Synthesis of Intermediate D113

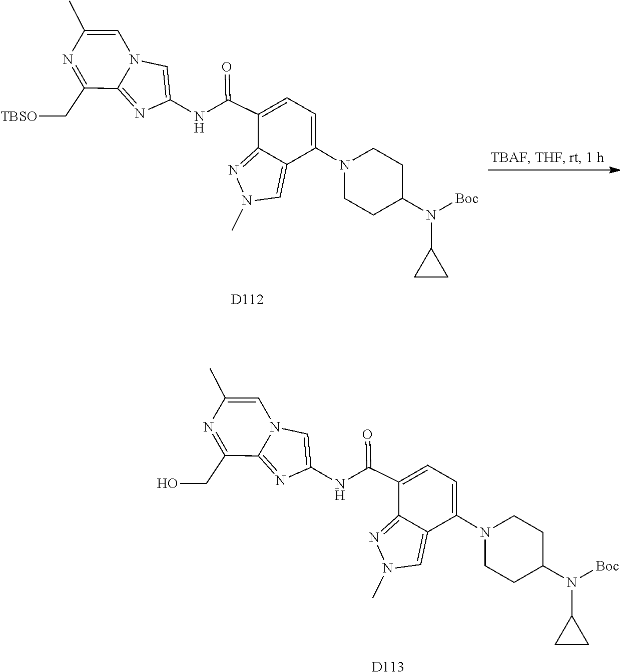 [SMILES: Cc1cn2cc(NC(=O)c3ccc(N4CCC(N(C(=O)OC(C)(C)C)C5CC5)CC4)c4cn(C)nc34)nc2c(CO[Si](C)(C)C(C)(C)C)n1 ; Cc1cn2cc(NC(=O)c3ccc(N4CCC(N(C(=O)OC(C)(C)C)C5CC5)CC4)c4cn(C)nc34)nc2c(CO)n1 ; "D112" ; "D113"]

To a stirred mixture of Intermediate D112 (180.0 mg, 261.3 μmol) in THF (4 mL) was added TBAF (109.5 mg, 391.9 μmol) at room temperature under N₂. The resulting mixture was stirred for 1 h, then quenched with water (10 mL) at room temperature, and subsequently extracted with EtOAc (2×10 mL). The combined organic layers were washed with brine (10 mL), dried over anhydrous Na₂SO₄, and filtered. The filtrate was concentrated under reduced pressure and purified by silica gel column chromatography (5% MeOH in DCM) to afford tert-butyl N-cyclopropyl-N-[1-[7-[[8-(hydroxymethyl)-6-methyl-imidazo[1,2-a]-pyrazin-2-yl]-carbamoyl]-2-methyl-indazol-4-yl]-4-piperidyl]carbamate (D113, 110 mg, 191.4 μmol). LCMS (ES, m/z): 575 [M+H]⁺.

### Synthesis of Intermediate D114

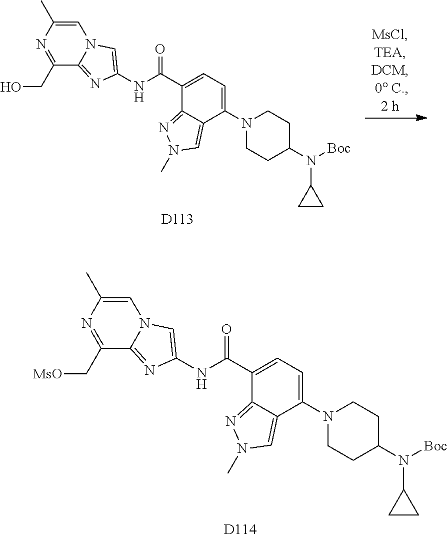

To a stirred mixture of Intermediate D113 (100.0 mg, 174 μmol) in DCM (2 mL) was added TEA (35.2 mg, 348 μmol) and MsCl (29.7 mg, 261 μmol) at 0° C. The resulting mixture was stirred for 2 h at 0° C., then quenched with water (10 mL) at 0° C., and subsequently extracted with EtOAc (2×10 mL). The combined organic layers were washed with brine (10 mL), dried over anhydrous Na₂SO₄, filtered, and the filtrate was concentrated under reduced pressure to afford [2-[[4-[4-[tert-butoxycarbonyl(cyclopropyl)amino]-1-piperidyl]-2-methyl-indazole-7-carbonyl]-amino]-6-methyl-imidazo[1,2-a]pyrazin-8-yl]methyl methanesulfonate (D114, 100 mg, 153.2 μmol). LCMS (ES, m/z): 653 [M+H]⁺.

### Synthesis of Intermediate D115

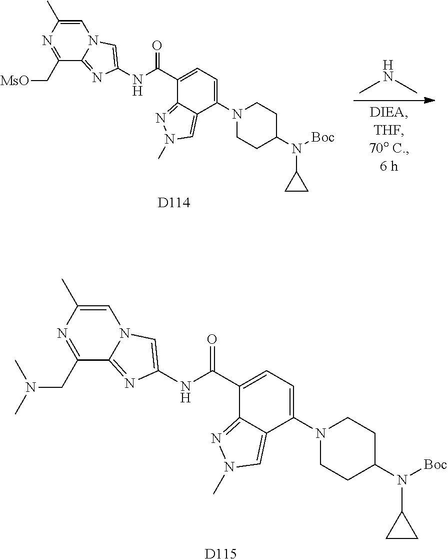

To a stirred mixture of Intermediate D114 (100.0 mg, 153.2 μmol) in THF (2 mL) was added DIEA (59.4 mg, 459.6 μmol) and N-methylmethanamine (27.6 mg, 612.8 μmol), and the reaction was stirred for 6 h at 70° C. The resulting mixture was diluted with water (10 mL) at room temperature, and extracted with EtOAc (2×10 mL). The combined organic layers were washed with brine (10 mL), dried over anhydrous Na₂SO₄, and filtered. The filtrate was concentrated under reduced pressure and purified by silica gel column chromatography (9% MeOH in DCM) to afford tert-butyl N-cyclopropyl-N-[1-[7-[[8-[(dimethylamino)methyl]-6-methyl-imidazo[1,2-a]pyrazin-2-yl]carbamoyl]-2-methyl-indazol-4-yl]-4-piperidyl]carbamate (D115, 70 mg, 116.3 μmol). LCMS (ES, m/z): 602 [M+H]⁺.

### Synthesis of Compound 600

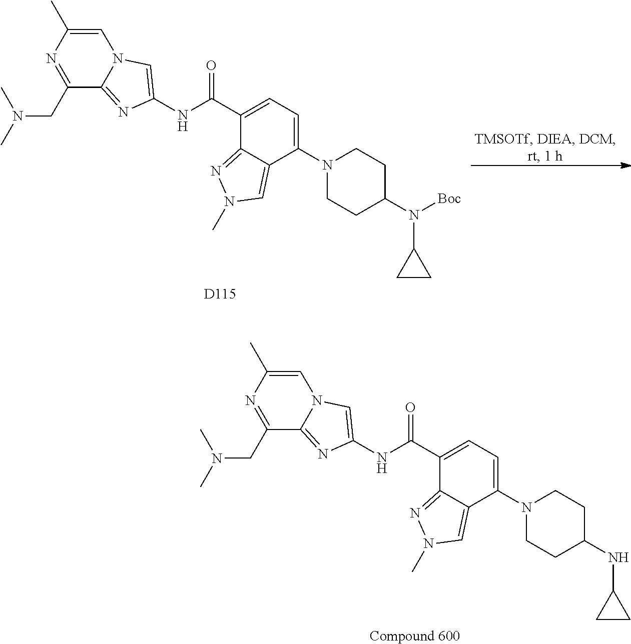 [SMILES: Cc1cn2cc(NC(=O)c3ccc(N4CCC(N(C(=O)OC(C)(C)C)C5CC5)CC4)c4cn(C)nc34)nc2c(CN(C)C)n1 ; "D115" ; Cc1cn2cc(NC(=O)c3ccc(N4CCC(NC5CC5)CC4)c4cn(C)nc34)nc2c(CN(C)C)n1 ; "Compound 600"]

To a solution of Intermediate D115 (70 mg, 116.3 μmol) in DCM (1 mL) were added DIEA (30 mg, 232.7 μmol) and TMSOTf (77.5 mg, 349 μmol), and the reaction was stirred for 1 h at room temperature. The resulting mixture was concentrated under reduced pressure and adjusted to pH 8 with saturated NaHCO₃ (aq.). The residue was purified by reversed-phase flash chromatography (Condition 10, Gradient 2) to afford 4-(4-(cyclopropylamino)piperidin-1-yl)-N-(8-((dimethylamino)methyl)-6-methylimidazo[1,2-a]pyrazin-2-yl)-2-methyl-2H-indazole-7-carboxamide bis(2,2,2-trifluoroacetate) (Compound 600, 24 mg, 32.9 μmol). LCMS (ES, m/z): 502 [M+H]⁺. ¹H NMR (400 MHz, DMSO-d₆) δ 11.54 (s, 1H), 10.03 (s, 1H), 8.90 (s, 1H), 8.86 (s, 2H), 8.57 (s, 1H), 8.51 (s, 1H), 8.08 (d, J=8.1 Hz, 1H), 6.59 (d, J=8.2 Hz, 1H), 4.92 (s, 2H), 4.30 (s, 3H), 4.07 (d, J=13.0 Hz, 2H), 3.50-3.39 (m, 3H), 3.31-3.25 (m, 1H), 3.07 (t, J=12.4 Hz, 2H), 2.99 (s, 6H), 2.80 (s, 1H), 2.22 (d, J=12.2 Hz, 2H), 1.76 (q, J=12.4 Hz, 2H), 0.85 (d, J=5.6 Hz, 4H).

### Example 218: Synthesis of D116

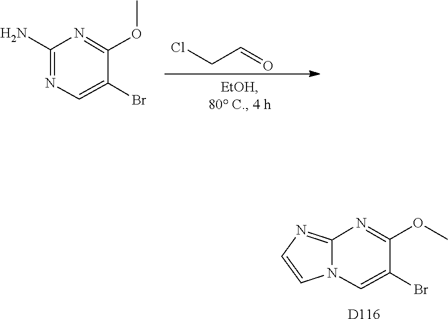

To a solution of 5-bromo-4-methoxy-pyrimidin-2-amine (500 mg, 2.5 mmol) in EtOH (5 mL) was added 2-chloroacetaldehyde (40% in H₂O) (721.4 mg, 3.7 mmol), and the reaction was stirred for 4 h at 80° C. The resulting mixture was diluted with H₂O (30 mL) and extracted with EtOAc (2×30 mL). The combined organic layers were washed with H₂O (2×30 mL), dried over anhydrous Na₂SO₄, and filtered. The filtrate was concentrated under reduced pressure and purified by silica gel column chromatography (20% EtOAc in DCM) to afford 6-bromo-7-methoxyimidazo[1,2-a]pyrimidine (D116, 490 mg, 2.2 mmol). LCMS (ES, m/z): 228 [M+H]⁺.

### Example 219: Synthesis of Compound 144

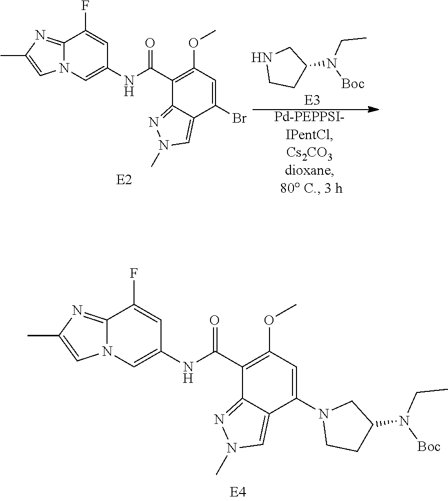

To a stirred mixture of 4-bromo-N-(8-fluoro-2-methyl-imidazo[1,2-a]pyridin-6-yl)-6-methoxy-2-methyl-indazole-7-carboxamide (300 mg, 694.05 μmol) and tert-butyl (R)-ethyl(pyrrolidin-3-yl)carbamate (178.48 mg, 832.86 μmol) in dioxane (5 mL) were added Pd-PEPPS-IPentCl (54.95 mg, 69.40 μmol) and CS₂CO₃ (678.40 mg, 2.08 mmol) at room temperature under nitrogen atmosphere. The resulting mixture was stirred for 3 h at 80° C. under nitrogen atmosphere. The mixture was allowed to cool down to room temperature. The resulting mixture was concentrated under reduced pressure. The residue was purified by silica gel column chromatography, eluted with DCM/MeOH (20:1) to afford tert-butyl N-ethyl-N-[1-[7-[(8-fluoro-2-methyl-imidazo[1,2-a]pyridin-6-yl)carbamoyl]-6-methoxy-2-methyl-indazol-4-yl]pyrrolidin-3-yl]carbamate (155 mg, 274.03 μmol, 40% yield) as a solid. LCMS (ES, m/z): 566 [M+H]⁺.

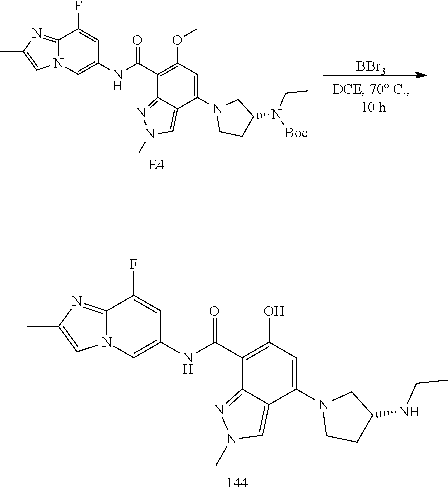

To a stirred solution of tert-butyl N-ethyl-N-[1-[7-[(8-fluoro-2-methyl-imidazo[1,2-a]pyridin-6-yl)carbamoyl]-6-methoxy-2-methyl-indazol-4-yl]pyrrolidin-3-yl]carbamate (155 mg, 274.03 μmol) in DCE (3 mL) was added tribromoborane (0.5 mL) in portions at 0° C. The resulting mixture was stirred for 10 h at 70° C. The reaction was quenched with MeOH at 0° C. and concentrated under reduced pressure. The residue was purified by reverse flash chromatography with the following conditions: column, C18 silica gel; mobile phase, MeCN in Water (0.1% NH₃·H₂O+10 mmol/L NH₄HCO₃), 30% to 80% gradient in 10 min; detector, UV 220 nm. This resulted in 4-[3-(ethylamino)pyrrolidin-1-yl]-N-(8-fluoro-2-methyl-imidazo[1,2-a]pyridin-6-yl)-6-hydroxy-2-methyl-indazole-7-carboxamide (49.9 mg, 110.52 μmol, 40% yield) as a solid. LCMS (ES, m/z): 452 [M+H]⁺. ¹H NMR (400 MHz, DMSO-d₆) δ 13.49 (s, 1H), 11.01 (s, 1H), 9.03 (d, J=1.7 Hz, 1H), 8.67 (s, 1H), 7.89 (d, J=3.1 Hz, 1H), 7.30 (d, J=12.2 Hz, 1H), 5.49 (s, 1H), 4.17 (s, 3H), 3.75 (s, 1H), 3.69 (s, 1H), 3.59 (s, 1H), 3.46 (s, 1H), 2.64 (q, J=7.8, 7.2 Hz, 2H), 2.35 (s, 3H), 2.19-2.11 (m, 1H), 1.95-1.86 (m, 1H), 1.05 (t, J=7.1 Hz, 3H).

### Example 220: Synthesis of Compound 113

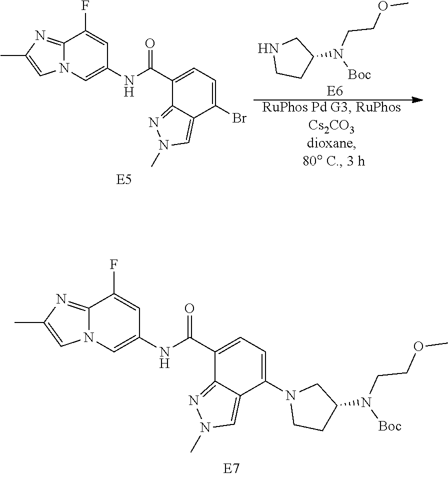

To a mixture of 4-bromo-N-(8-fluoro-2-methyl-imidazo[1,2-a]pyridin-6-yl)-2-methyl-indazole-7-carboxamide (100 mg, 248.62 μmol) and tert-butyl N-(2-methoxyethyl)-N-[(3R)-pyrrolidin-3-yl]carbamate (73 mg, 298.34 μmol) in Dioxane (3 mL) were added Cs₂CO₃ (162 mg, 497.24 μmol), RuPhos Pd G3 (21 mg, 24.86 μmol) and Ruphos (23 mg, 49.72 μmol). The resulting mixture was stirred for 4 hr at 90° C. under nitrogen atmosphere. The resulting mixture was concentrated under reduced pressure. The residue was purified by silica gel column chromatography, eluted with PE/EA (1:2) to afford tert-butyl N-[(3R)-1-[7-[(8-fluoro-2-methyl-imidazo[1,2-a]pyridin-6-yl)carbamoyl]-2-methyl-indazol-4-yl]pyrrolidin-3-yl]-N-(2-methoxyethyl)carbamate (80 mg, 141.43 μmol, 57% yield) as a solid. LCMS (ES, m/z): 566 [M+H]⁺.

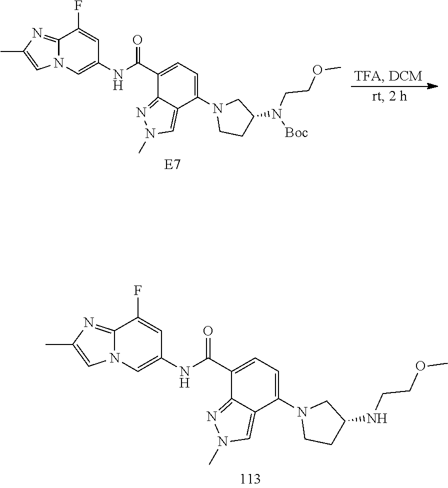

To a stirred solution of tert-butyl N-[(3R)-1-[7-[(8-fluoro-2-methyl-imidazo[1,2-a]pyridin-6-yl)carbamoyl]-2-methyl-indazol-4-yl]pyrrolidin-3-yl]-N-(2-methoxyethyl)carbamate (80 mg, 141.43 μmol) in DCM (2 mL) was added TFA (1 mL, 13.07 mmol) at rt. The resulting mixture was stirred for 2 hr. The resulting mixture was concentrated under reduced pressure. The crude product was purified by Prep-HPLC with the following conditions (SunFire Prep C18 OBD Column 19*150 mm, 5 Um 10 nm, mobile phase, MeCN in water (0.05% NH₄OH), 15% to 25% gradient in 7 min; detector, UV 254 nm) to afford N-(8-fluoro-2-methyl-imidazo[1,2-a]pyridin-6-yl)-4-[(3R)-3-(2-methoxyethylamino)pyrrolidin-1-yl]-2-methyl-indazole-7-carboxamide (39.9 mg, 85.71 μmol, 61% yield) as a solid. LCMS (ES, m/z): 466 [M+H]⁺. ¹H NMR (400 MHz, DMSO-d₆) δ 11.01 (s, 1H), 9.20 (d, J=1.6 Hz, 1H), 8.83 (s, 1H), 7.96-7.86 (m, 2H), 7.31 (dd, J=12.4, 1.6 Hz, 1H), 6.01 (d, J=8.4 Hz, 1H), 4.27 (s, 3H), 3.80 (d, J=7.9 Hz, 1H), 3.73 (d, J=8.5 Hz, 1H), 3.63 (d, J=8.1 Hz, 1H), 3.52-3.38 (m, 4H), 3.28 (s, 3H), 2.77 (t, J=5.7 Hz, 2H), 2.35 (s, 3H), 2.17 (dt, J=12.8, 6.2 Hz, 1H), 1.91 (dd, J=12.0, 6.2 Hz, 1H).

### Example 221: Synthesis of Compound 465

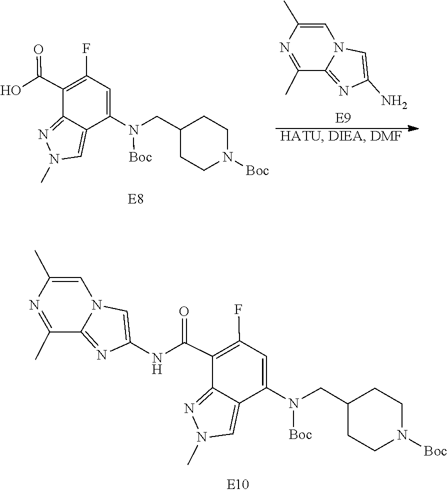 [SMILES: Cn1cc2c(N(CC3CCN(C(=O)OC(C)(C)C)CC3)C(=O)OC(C)(C)C)cc(F)c(C(=O)O)c2n1 ; Cc1cn2cc(N)nc2c(C)n1 ; Cc1cn2cc(NC(=O)c3c(F)cc(N(CC4CCN(C(=O)OC(C)(C)C)CC4)C(=O)OC(C)(C)C)c4cn(C)nc34)nc2c(C)n1]

To a stirred mixture of 4-[tert-butoxycarbonyl-[(1-tert-butoxycarbonyl-4-piperidyl)methyl]amino]-6-fluoro-2-methyl-indazole-7-carboxylic acid (100 mg, 197.41 μmol) and 6,8-dimethylimidazo[1,2-a]pyrazin-2-amine dihydrochloride (55.4 mg, 236.89 μmol) in DMF (0.5 mL) were added DIEA (102.0 mg, 789.63 μmol) and HATU (187.6 mg, 493.52 μmol) at room temperature. The resulting mixture was stirred for 3 h at rt. The resulting mixture was extracted with EA (2×10 mL). The combined organic layers were washed with water (2×100 mL), dried over anhydrous Na₂SO₄. After filtration, the filtrate was concentrated under reduced pressure. The residue was purified by silica gel column chromatography, eluted with PE/EA (1:2) to afford tert-butyl 4-[[tert-butoxycarbonyl-[7-[(6,8-dimethylimidazo[1,2-a]pyrazin-2-yl)carbamoyl]-6-fluoro-2-methyl-indazol-4-yl]amino]methyl]piperidine-1-carboxylate (70 mg, 107.57 μmol, 54% yield) as a solid. LCMS (ES, m/z): 650 [M+H]⁺.

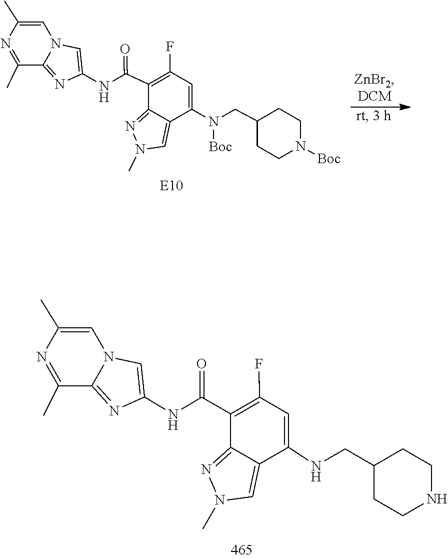 [SMILES: Cc1cn2cc(NC(=O)c3c(F)cc(N(CC4CCN(C(=O)OC(C)(C)C)CC4)C(=O)OC(C)(C)C)c4cn(C)nc34)nc2c(C)n1 ; Cc1cn2cc(NC(=O)c3c(F)cc(NCC4CCNCC4)c4cn(C)nc34)nc2c(C)n1]

To a stirred mixture of tert-butyl 4-[[tert-butoxycarbonyl-[7-[(6,8-dimethylimidazo[1,2-a]pyrazin-2-yl)carbamoyl]-6-fluoro-2-methyl-indazol-4-yl]amino]methyl]piperidine-1-carboxylate (50 mg, 76.84 μmol) in DCM (2 mL) was added ZnBr₂ (173.0 mg, 768.35 μmol) at room temperature. The resulting mixture was stirred for 2 h at room temperature. The resulting mixture were concentrated under reduced pressure and diluted with water (2 mL). The mixture was basified to pH 8 with saturated NaHCO₃ (aq.) and extracted with DCM (3×3 mL). The combined organic layers were washed with water (2×10 mL) and brine (1×10 mL), dried over anhydrous Na₂SO₄. After filtration, the filtrate was concentrated under reduced pressure. The crude product was purified by Prep-HPLC with the following conditions (column, C18 silica gel, XBridge, 19×150 mm; mobile phase, MeCN in water (0.05% NH₃H₂O), 25% to 40% gradient in 7 min; detector, UV 254 nm) to afford N-(6,8-dimethylimidazo[1,2-a]pyrazin-2-yl)-6-fluoro-2-methyl-4-(4-piperidylmethylamino)indazole-7-carboxamide (5 mg, 11.10 μmol, 14% yield) as a solid. LCMS (ES, m/z): 450 [M+H]⁺. ¹H NMR (400 MHz, Methanol-d₄) δ 8.39 (s, 1H), 8.31 (s, 1H), 8.21 (s, 1H), 6.02 (d, J=16.0 Hz, 1H), 4.31 (s, 3H), 3.40-3.35 (m, 2H), 3.32-3.29 (m, 2H), 2.92 (t, J=12.9 Hz, 2H), 2.81 (s, 3H), 2.48 (d, J=1.0 Hz, 3H), 2.10-2.01 (m, 3H), 1.57-1.39 (m, 2H).

### Example 222: Synthesis of Compound 688

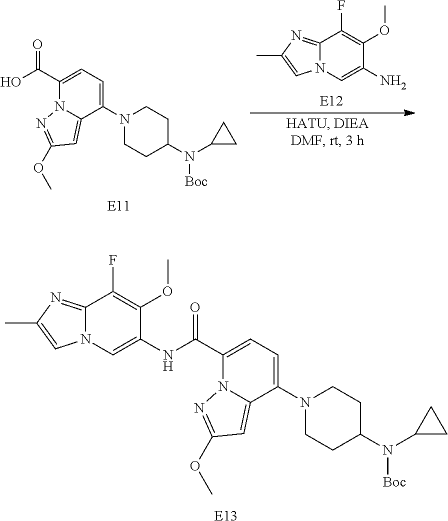

To a solution of 4-[4-[tert-butoxycarbonyl(cyclopropyl)amino]-1-piperidyl]-2-methoxy-pyrazolo[1,5-a]pyridine-7-carboxylic acid (60 mg, 139.37 μmol) in DMF (0.6 mL) were added 8-fluoro-7-methoxy-2-methyl-imidazo[1,2-a]pyridin-6-amine (32.6 mg, 167.25 μmol), DIEA (72.0 mg, 557.50 μmol) and HATU (79.5 mg, 209.06 μmol). The mixture was stirred for 3 h at rt. The reaction was diluted with H₂O and the precipitated solids were collected by filtration to afford tert-butyl N-cyclopropyl-N-[1-[7-[(8-fluoro-7-methoxy-2-methyl-imidazo[1,2-a]pyridin-6-yl)carbamoyl]-2-methoxy-pyrazolo[1,5-a]pyridin-4-yl]-4-piperidyl]carbamate (80 mg, 131.65 μmol, 94% yield) as a solid. LCMS (ES, m/z): 608 [M+H]⁺.

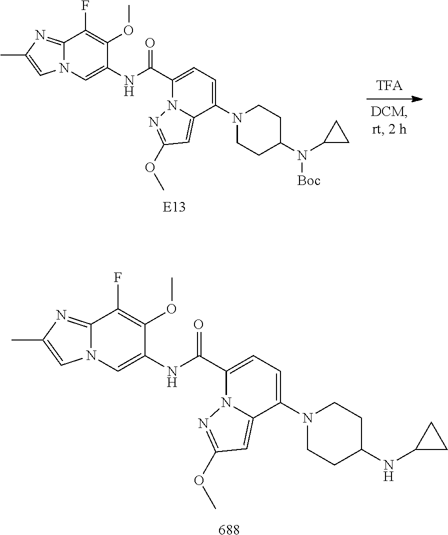

To a solution of tert-butyl N-cyclopropyl-N-[1-[7-[(8-fluoro-7-methoxy-2-methyl-imidazo[1,2-a]pyridin-6-yl)carbamoyl]-2-methoxy-pyrazolo[1,5-a]pyridin-4-yl]-4-piperidyl]carbamate (75 mg, 123.42 μmol) in DCM (0.8 mL) was added TFA (188 μL) at rt. The mixture was stirred for 2 h at rt. The resulting mixture was concentrated under reduced pressure. Then diluted with sat NaHCO₃ (aq.) (10 mL). The resulting mixture was extracted with DCM (2×10 mL). The combined organic layers were washed with H₂O (10 mL), dried over anhydrous Na₂SO₄. After filtration, the filtrate was concentrated under reduced pressure. The crude product was purified by Prep-HPLC with the following conditions (column, C18 silica gel, XBridge, 19×150 mm; mobile phase, MeCN in water (0.05% NH₃H₂O), 50% to 70% gradient in 7 min; detector, UV 254 nm) to afford 4-[4-(cyclopropylamino)-1-piperidyl]-N-(8-fluoro-7-methoxy-2-methyl-imidazo[1,2-a]pyridin-6-yl)-2-methoxy-pyrazolo[1,5-a]pyridine-7-carboxamide (45 mg, 88.66 μmol, 72% yield) as a solid. LCMS (ES, m/z): 508 [M+H]⁺. ¹H NMR (400 MHz, DMSO-d₆) δ 12.69 (s, 1H), 9.41 (d, J=1.2 Hz, 1H), 7.79 (d, J=3.0 Hz, 1H), 7.74 (d, J=8.1 Hz, 1H), 6.77 (d, J=8.5 Hz, 1H), 6.26 (s, 1H), 4.12 (d, J=2.1 Hz, 3H), 4.06 (s, 3H), 3.65 (d, J=12.2 Hz, 2H), 2.88 (t, J=11.2 Hz, 2H), 2.73 (dd, J=9.3, 5.0 Hz, 1H), 2.35-2.25 (m, 4H), 2.10 (tt, J=6.8, 3.6 Hz, 1H), 2.02-1.95 (m, 2H), 1.49 (d, J=11.2 Hz, 2H), 0.37 (dt, J=6.1, 3.0 Hz, 2H), 0.21 (p, J=3.9 Hz, 2H).

### Example 223: Synthesis of Compound 689

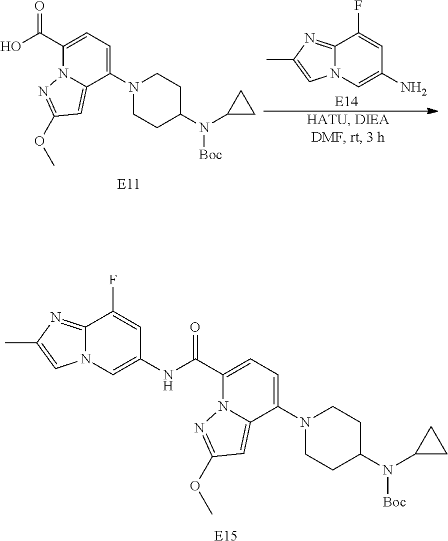

To a stirred mixture of 4-[4-[tert-butoxycarbonyl(cyclopropyl)amino]-1-piperidyl]-2-methoxy-pyrazolo[1,5-a]pyridine-7-carboxylic acid (70 mg, 162.60 μmol) and 8-fluoro-2-methyl-imidazo[1,2-a]pyridin-6-amine (32.2 mg, 195.12 μmol) in DMF (1.5 mL) were added HATU (92.7 mg, 243.90 μmol) and DIEA (63.0 mg, 487.81 μmol) at room temperature. The resulting mixture was stirred for 3 h at rt. The resulting mixture was extracted with EA (1×5 mL). The combined organic layers were washed with water (2×10 mL), dried over anhydrous Na₂SO₄. After filtration, the filtrate was concentrated under reduced pressure. The residue was purified by silica gel column chromatography, eluted with PE/EA (1:2) to afford tert-butyl N-cyclopropyl-N-[1-[7-[(8-fluoro-2-methyl-imidazo[1,2-a]pyridin-6-yl)carbamoyl]-2-methoxy-pyrazolo[1,5-a]pyridin-4-yl]-4-piperidyl]carbamate (70 mg, 121.18 μmol, 75% yield) as a solid. LCMS (ES, m/z): 578 [M+H]⁺.

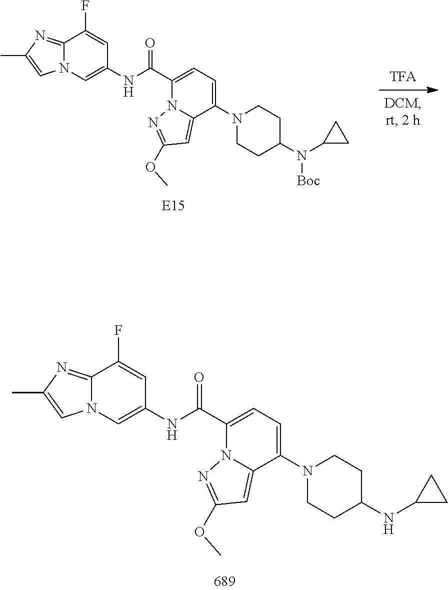 [SMILES: COc1cc2c(N3CCC(N(C(=O)OC(C)(C)C)C4CC4)CC3)ccc(C(=O)Nc3cc(F)c4nc(C)cn4c3)n2n1 ; COc1cc2c(N3CCC(NC4CC4)CC3)ccc(C(=O)Nc3cc(F)c4nc(C)cn4c3)n2n1]

To a stirred solution of tert-butyl N-cyclopropyl-N-[1-[7-[(8-fluoro-2-methyl-imidazo[1,2-a]pyridin-6-yl)carbamoyl]-2-methoxy-pyrazolo[1,5-a]pyridin-4-yl]-4-piperidyl]carbamate (70 mg, 121.18 μmol) in DCM (2 mL) was added TFA (1 mL) at rt. The resulting mixture was stirred for 2 h. The resulting mixture was concentrated under reduced pressure. The crude product was purified by Prep-HPLC with the following conditions (SunFire Prep C18 OBD Column 19*150 mm, 5 Um 10 nm, mobile phase, MeCN in water (0.05% NH₄OH), 50% to 80% gradient in 7 min; detector, UV 254 nm) to afford 4-[4-(cyclopropylamino)-1-piperidyl]-N-(8-fluoro-2-methyl-imidazo[1,2-a]pyridin-6-yl)-2-methoxy-pyrazolo[1,5-a]pyridine-7-carboxamide (21.6 mg, 45.23 μmol, 37% yield) as a solid. LCMS (ES, m/z): 478 [M+H]⁺. ¹H NMR (400 MHz, DMSO-d₆) δ 12.13 (s, 1H), 9.17 (d, J=1.6 Hz, 1H), 7.94 (d, J=3.3 Hz, 1H), 7.66 (d, J=8.1 Hz, 1H), 7.26 (dd, J=12.3, 1.8 Hz, 1H), 6.77 (d, J=8.2 Hz, 1H), 6.24 (s, 1H), 4.09 (s, 3H), 3.66 (d, J=12.4 Hz, 2H), 2.90 (t, J=11.6 Hz, 2H), 2.76 (s, 1H), 2.36 (s, 3H), 2.16-2.10 (m, 1H), 2.01 (d, J=11.9 Hz, 2H), 1.54 (d, J=10.7 Hz, 1H), 1.48 (d, J=11.3 Hz, 1H), 1.24 (s, 1H), 0.40 (td, J=6.3, 4.0 Hz, 2H), 0.25 (q, J=3.4 Hz, 2H).

### Example 224: Synthesis of Compound 690

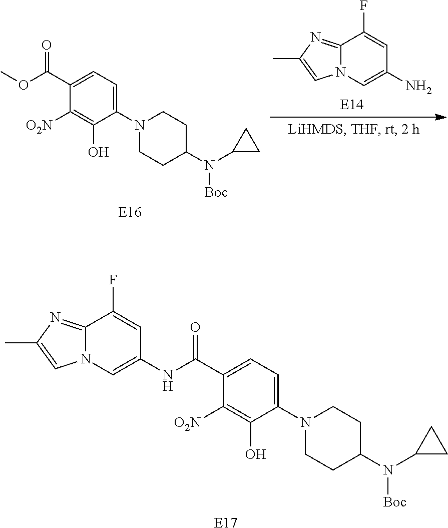 [SMILES: COC(=O)c1ccc(N2CCC(N(C(=O)OC(C)(C)C)C3CC3)CC2)c(O)c1[N+](=O)[O-] ; Cc1cn2cc(N)cc(F)c2n1 ; "E16" ; Cc1cn2cc(NC(=O)c3ccc(N4CCC(N(C(=O)OC(C)(C)C)C5CC5)CC4)c(O)c3[N+](=O)[O-])cc(F)c2n1 ; "E17"]

To a solution of methyl 4-[4-[tert-butoxycarbonyl(cyclopropyl)amino]-1-piperidyl]-3-hydroxy-2-nitro-benzoate (110 mg, 252.60 μmol) in THF (1.1 mL) was added 8-fluoro-2-methyl-imidazo[1,2-a]pyridin-6-amine (54.2 mg, 328.38 μmol) at rt. Then LiHMDS (1 mL, 1 mol/L in THF) was added dropwise at 0° C. under N₂. The mixture was stirred for 2 h at rt. The reaction was quenched with NH₄Cl (aq.). The resulting mixture was extracted with EA (2×10 mL). The combined organic layers were washed with H₂O (10 mL), dried over anhydrous Na₂SO₄. After filtration, the filtrate was concentrated under reduced pressure. The residue was purified by silica gel column chromatography, eluted with PE/EA (1/9) to afford tert-butyl N-cyclopropyl-N-[1-[4-[(8-fluoro-2-methyl-imidazo[1,2-a]pyridin-6-yl)carbamoyl]-2-hydroxy-3-nitro-phenyl]-4-piperidyl]carbamate (70 mg, 123.11 μmol, 49% yield) as an oil. LCMS (ES, m/z): 569 [M+H]⁺.

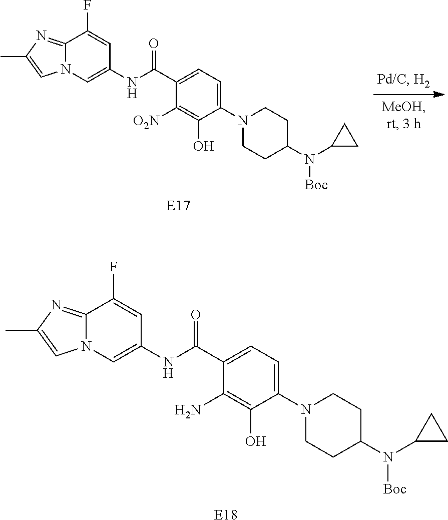 [SMILES: Cc1cn2cc(NC(=O)c3ccc(N4CCC(N(C(=O)OC(C)(C)C)C5CC5)CC4)c(O)c3[N+](=O)[O-])cc(F)c2n1 ; "E17" ; Cc1cn2cc(NC(=O)c3ccc(N4CCC(N(C(=O)OC(C)(C)C)C5CC5)CC4)c(O)c3N)cc(F)c2n1 ; "E18"]

To a solution of tert-butyl N-cyclopropyl-N-[1-[4-[(8-fluoro-2-methyl-imidazo[1,2-a]pyridin-6-yl)carbamoyl]-2-hydroxy-3-nitro-phenyl]-4-piperidyl]carbamate (70 mg, 123.11 μmol) in MeOH (15 mL) was added Pd/C (10% on Carbon (wetted with ca. 55% water)) (26.2 mg, 246.22 μmol) at rt under N₂. The mixture was stirred for 3 h at rt under H₂. The mixture was filtered through a Celite pad and concentrated under reduced pressure to afford tert-butyl N-[1-[3-amino-4-[(8-fluoro-2-methyl-imidazo[1,2-a]pyridin-6-yl)carbamoyl]-2-hydroxy-phenyl]-4-piperidyl]-N-cyclopropyl-carbamate (58 mg, 107.68 μmol, 87% yield) as an oil. LCMS (ES, m/z): 539 [M+H]⁺.

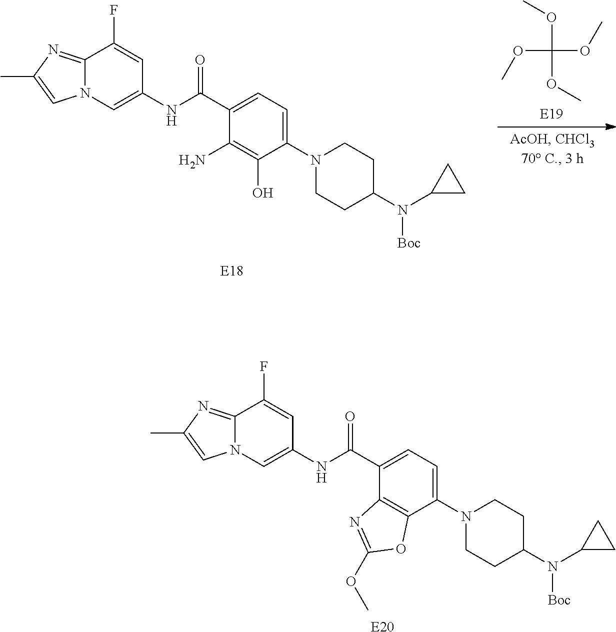

To a solution of tert-butyl N-[1-[3-amino-4-[(8-fluoro-2-methyl-imidazo[1,2-a]pyridin-6-yl)carbamoyl]-2-hydroxy-phenyl]-4-piperidyl]-N-cyclopropyl-carbamate (57 mg, 105.83 μmol) in CHCl₃ (0.6 mL) were added tetramethoxymethane (28.8 mg, 211.66 μmol) and Acetic acid (25.4 mg, 423.31 μmol). The mixture was stirred for 3 h at 70° C. The resulting mixture was diluted with H₂O and adjusted to pH 8 with NaHCO₃ (aq.). The resulting mixture was extracted with DCM (2×10 mL). The combined organic layers were washed with H₂O (10 mL), dried over anhydrous Na₂SO₄. After filtration, the filtrate was concentrated under reduced pressure. The residue was purified by silica gel column chromatography, eluted with DCM/EA (2:3) to afford tert-butyl N-cyclopropyl-N-[1-[4-[(8-fluoro-2-methyl-imidazo[1,2-a]pyridin-6-yl)carbamoyl]-2-methoxy-1,3-benzoxazol-7-yl]-4-piperidyl]carbamate (50 mg, 86.41 μmol, 82% yield) as a solid. LCMS (ES, m/z): 579 [M+H]⁺.

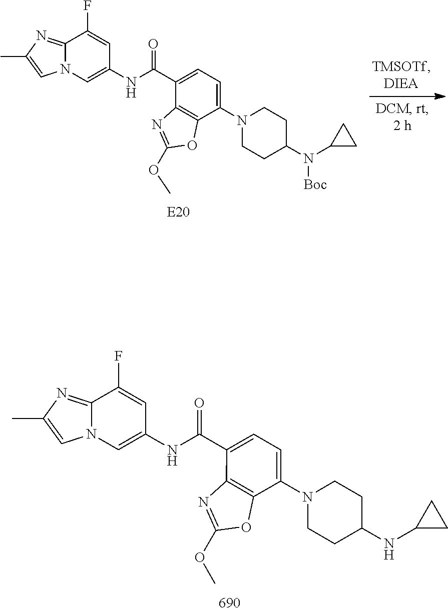 [SMILES: COc1nc2c(C(=O)Nc3cc(F)c4nc(C)cn4c3)ccc(N3CCC(N(C(=O)OC(C)(C)C)C4CC4)CC3)c2o1 ; COc1nc2c(C(=O)Nc3cc(F)c4nc(C)cn4c3)ccc(N3CCC(NC4CC4)CC3)c2o1]

To a solution of tert-butyl N-cyclopropyl-N-[1-[4-[(8-fluoro-2-methyl-imidazo[1,2-a]pyridin-6-yl)carbamoyl]-2-methoxy-1,3-benzoxazol-7-yl]-4-piperidyl]carbamate (50 mg, 86.41 μmol) in DCM (0.5 mL) were added DIEA (22.3 mg, 172.82 μmol) and TMSOTf (57.6 mg, 259.23 μmol). The mixture was stirred for 2 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃·H₂O), 40% to 70% gradient in 7 min; detector, UV 254 nm) to afford 7-[4-(cyclopropylamino)-1-piperidyl]-N-(8-fluoro-2-methyl-imidazo[1,2-a]pyridin-6-yl)-2-methoxy-1,3-benzoxazole-4-carboxamide (15 mg, 31.35 μmol, 36% yield) as a solid. LCMS (ES, m/z): 479 [M+H]⁺. ¹H NMR (400 MHz, DMSO-d₆) δ 10.51 (s, 1H), 9.19 (d, J=1.6 Hz, 1H), 7.91 (d, J=3.1 Hz, 1H), 7.80 (d, J=8.9 Hz, 1H), 7.28 (dd, J=12.3, 1.7 Hz, 1H), 6.88 (d, J=8.9 Hz, 1H), 4.39 (s, 3H), 3.90 (d, J=12.9 Hz, 2H), 3.06 (t, J=11.5 Hz, 2H), 2.79 (q, J=12.3, 10.3 Hz, 1H), 2.35 (s, 3H), 2.13 (dt, J=6.7, 3.2 Hz, 1H), 1.97 (d, J=12.8 Hz, 2H), 1.41 (q, J=11.6, 9.7 Hz, 2H), 0.44-0.35 (m, 2H), 0.26-0.20 (m, 2H).

### Example 225: Synthesis of Compound 691

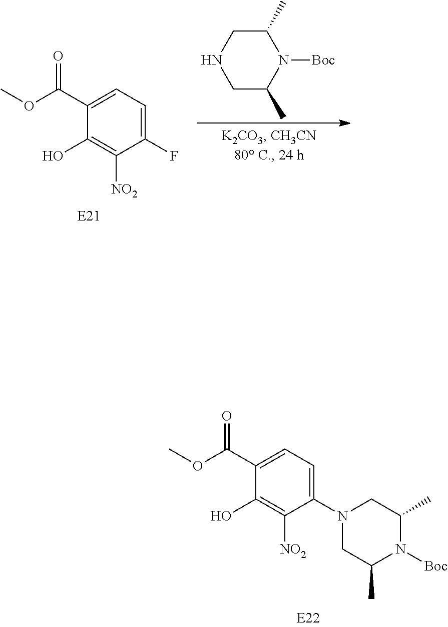 [SMILES: COC(=O)c1ccc(F)c([N+](=O)[O-])c1O ; C[C@H]1CNC[C@H](C)N1C(=O)OC(C)(C)C ; COC(=O)c1ccc(N2C[C@H](C)N(C(=O)OC(C)(C)C)[C@@H](C)C2)c([N+](=O)[O-])c1O]

To a stirred mixture of methyl 4-fluoro-2-hydroxy-3-nitro-benzoate (2 g, 9.30 mmol) and tert-butyl (2S,6S)-2,6-dimethylpiperazine-1-carboxylate (2.39 g, 11.16 mmol) in CH₃CN (40 mL) were added K₂CO₃ (3.85 g, 27.89 mmol) at room temperature. The resulting mixture was stirred for 24 h at 80° C. The mixture was diluted with water (80 mL) and extracted with EA (2×80 mL). The combined organic layers were washed with water (100 mL) and dried by anhydrous Na₂SO₄. The resulting organic layer was concentrated under reduced pressure. The residue was purified by silica gel column chromatography, eluted with PE/EA (1:2) to afford tert-butyl (2S,6S)-4-(3-hydroxy-4-methoxycarbonyl-2-nitro-phenyl)-2,6-dimethyl-piperazine-1-carboxylate (680 mg, 1.66 mmol, 18% yield) as a solid. LCMS (ES, m/z): 410 [M+H]⁺.

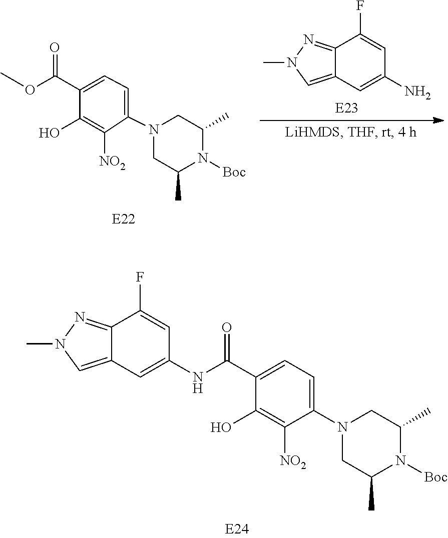 [SMILES: COC(=O)c1ccc(N2C[C@H](C)N(C(=O)OC(C)(C)C)[C@@H](C)C2)c([N+](=O)[O-])c1O ; Cn1cc2cc(N)cc(F)c2n1 ; C[C@H]1CN(c2ccc(C(=O)Nc3cc(F)c4nn(C)cc4c3)c(O)c2[N+](=O)[O-])C[C@H](C)N1C(=O)OC(C)(C)C]

To a stirred mixture of tert-butyl (2S,6S)-4-(3-hydroxy-4-methoxycarbonyl-2-nitro-phenyl)-2,6-dimethyl-piperazine-1-carboxylate (680 mg, 1.66 mmol) and 7-fluoro-2-methyl-indazol-5-amine (329.1 mg, 1.99 mmol) in THF (14 mL) was treated with LiHMDS (833.7 mg, 4.98 mmol) at 0° C. under nitrogen atmosphere. The resulting mixture was stirred for 4 h at room temperature. The resulting mixture was diluted with cold water (10 mL). The mixture was neutralized to pH 5 with HCl (aq.). The aqueous layer was extracted with EA (2×10 mL). The resulting mixture was concentrated under reduced pressure afford tert-butyl (2S,6S)-4-[4-[(7-fluoro-2-methyl-indazol-5-yl)carbamoyl]-3-hydroxy-2-nitro-phenyl]-2,6-dimethyl-piperazine-1-carboxylate (600 mg, 1.11 mmol, 67% yield) as a solid. LCMS (ES, m/z): 543 [M+H].

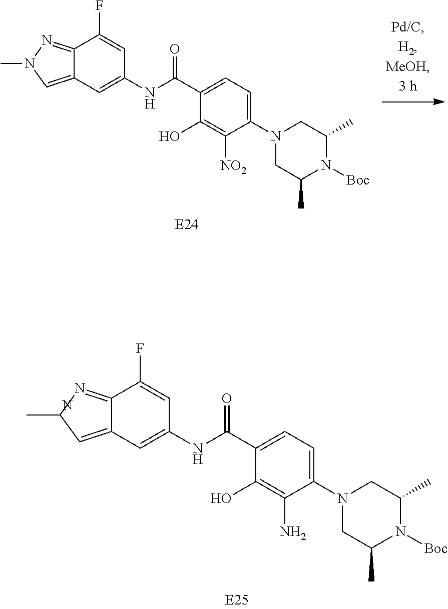

To a stirred mixture of tert-butyl (2S,6S)-4-[4-[(7-fluoro-2-methyl-indazol-5-yl)carbamoyl]-3-hydroxy-2-nitro-phenyl]-2,6-dimethyl-piperazine-1-carboxylate (600 mg, 1.11 mmol) in MeOH (12 mL) was treated with Pd/C (117.6 mg) at rt. The resulting mixture was stirred for 2 h at room temperature under hydrogen atmosphere. The resulting mixture was filter out Pd/C and concentrated under reduced pressure afford tert-butyl (2S,6S)-4-[2-amino-4-[(7-fluoro-2-methyl-indazol-5-yl)carbamoyl]-3-hydroxy-phenyl]-2,6-dimethyl-piperazine-1-carboxylate (500 mg, 975.47 μmol, 88.21% yield) as a solid. LCMS (ES, m/z): 513 [M+H]⁺.

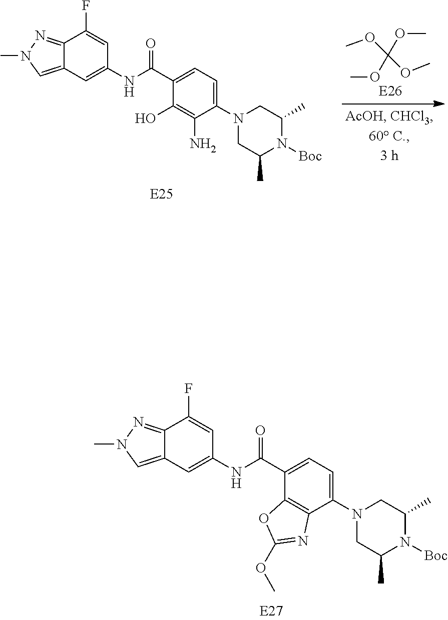

To a stirred mixture tert-butyl (2S,6S)-4-[2-amino-4-[(7-fluoro-2-methyl-indazol-5-yl)carbamoyl]-3-hydroxy-phenyl]-2,6-dimethyl-piperazine-1-carboxylate (500 mg, 975.47 μmol) and tetramethoxymethane (265.6 mg, 1.95 mmol) in CHCl₃ (10 mL) was added AcOH (175.5 mg, 2.93 mmol) in portions at room temperature. The resulting mixture was stirred for 3 h at 60° C. The mixture was acidified to pH 8 with saturated NaHCO₃ (aq.). The resulting mixture was extracted with EA (3×30 mL). The combined organic layers were washed with brine (1×30 mL), dried over anhydrous Na₂SO₄. After filtration, the filtrate was concentrated under reduced pressure. The residue was purified by silica gel column chromatography, eluted with PE/EA (1:1) to afford tert-butyl (2S,6S)-4-[7-[(7-fluoro-2-methyl-indazol-5-yl)carbamoyl]-2-methoxy-1,3-benzoxazol-4-yl]-2,6-dimethyl-piperazine-1-carboxylate (230 mg, 416.22 μmol, 43% yield) as a solid. LCMS (ES, m/z): 553 [M+H]⁺.

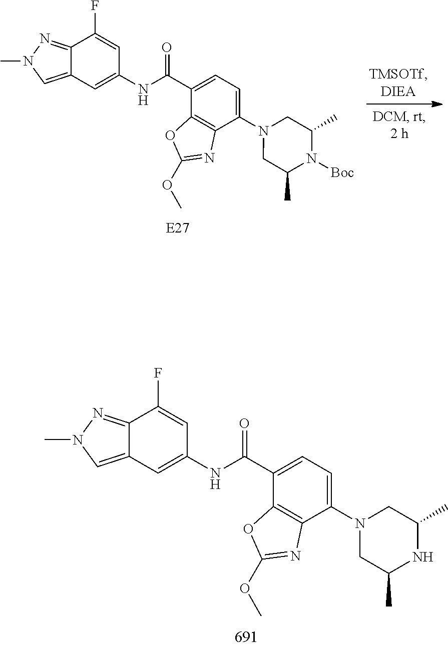

To a stirred mixture of tert-butyl (2S,6S)-4-[7-[(7-fluoro-2-methyl-indazol-5-yl)carbamoyl]-2-methoxy-1,3-benzoxazol-4-yl]-2,6-dimethyl-piperazine-1-carboxylate (70 mg, 126.67 μmol) in DCM (1.5 mL) was added DIEA (32.7 mg, 253.35 μmol) and TMSOTf (84.4 mg, 380.02 μmol) dropwise at room temperature. The resulting mixture was stirred for 2 h at room temperature. The resulting mixture were concentrated under reduced pressure and diluted with water (2 mL). The mixture was basified to pH 8 with saturated NaHCO₃ (aq.). The crude product was purified by Prep-HPLC with the following conditions (column, C18 silica gel, XBridge, 19×150 mm; mobile phase, MeCN in water (0.05% NH₃·H₂O), 30% to 50% gradient in 7 min; detector, UV 254 nm) to afford 4-[(3S,5S)-3,5-dimethylpiperazin-1-yl]-N-(7-fluoro-2-methyl-indazol-5-yl)-2-methoxy-1,3-benzoxazole-7-carboxamide (17.8 mg, 39.34 μmol, 31% yield) as a solid. LCMS (ES, m/z): 453 [M+H]⁺. ¹H NMR (400 MHz, DMSO-d₆) δ 9.93 (s, 1H), 8.43 (d, J=2.8 Hz, 1H), 8.00 (d, J=1.6 Hz, 1H), 7.55 (d, J=8.7 Hz, 1H), 7.40-7.32 (m, 1H), 6.76 (d, J=8.8 Hz, 1H), 4.19 (d, J=6.3 Hz, 6H), 3.53 (dd, J=11.9, 3.2 Hz, 2H), 3.36 (d, J=6.2 Hz, 2H), 3.23-3.22 (m, 2H), 1.12 (d, J=6.4 Hz, 6H).

### Example 226: Synthesis of Compound 692

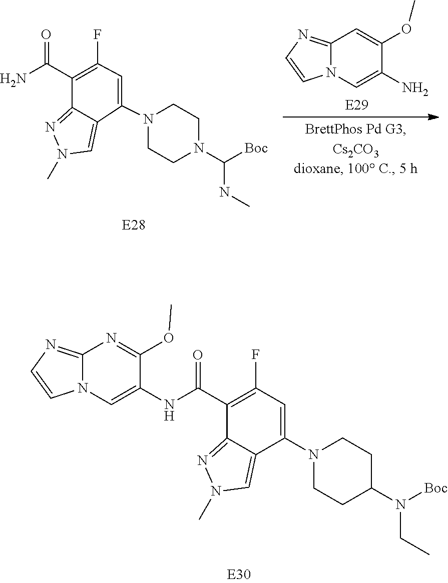 [SMILES: CNC(C(=O)OC(C)(C)C)N1CCN(c2cc(F)c(C(N)=O)c3nn(C)cc23)CC1 ; COc1cc2nccn2cc1N ; CCN(C(=O)OC(C)(C)C)C1CCN(c2cc(F)c(C(=O)Nc3cn4ccnc4nc3OC)c3nn(C)cc23)CC1]

To a stirred mixture of tert-butyl N-[1-(7-carbamoyl-6-fluoro-2-methyl-indazol-4-yl)-4-piperidyl]-N-ethyl-carbamate (70 mg, 166.87 μmol) and 6-bromo-7-methoxy-imidazo[1,2-a]pyrimidine (45.6 mg, 200.24 μmol) in Dioxane (1.5 mL) were added Cs₂CO₃ (108.7 mg, 333.74 μmol) and BrettPhos Pd G3 (15.1 mg, 16.69 μmol) at room temperature under nitrogen atmosphere. The resulting mixture was stirred for 5 h at 100° C. under nitrogen atmosphere. The resulting mixture was concentrated under reduced pressure. The residue was purified by silica gel column chromatography, eluted with PE/EA (1:3) to afford tert-butyl N-ethyl-N-[1-[6-fluoro-7-[(7-methoxyimidazo[1,2-a]pyrimidin-6-yl)carbamoyl]-2-methyl-indazol-4-yl]-4-piperidyl]carbamate (70 mg, 123.54 μmol, 74% yield) as a solid. LCMS (ES, m/z): 567 [M+H]⁺.

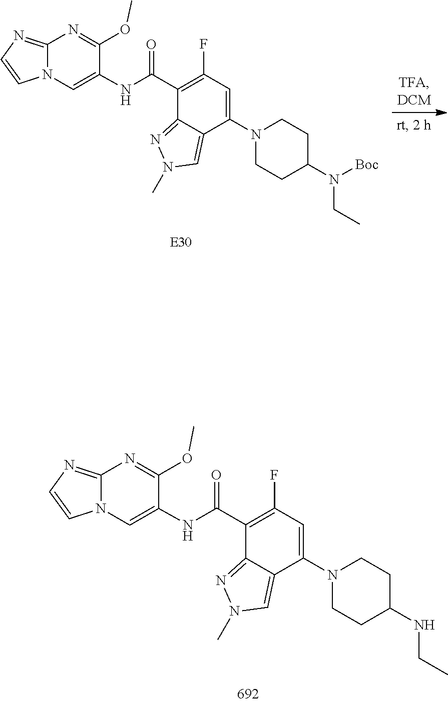 [SMILES: CCN(C(=O)OC(C)(C)C)C1CCN(c2cc(F)c(C(=O)Nc3cn4ccnc4nc3OC)c3nn(C)cc23)CC1 ; CCNC1CCN(c2cc(F)c(C(=O)Nc3cn4ccnc4nc3OC)c3nn(C)cc23)CC1]

To a stirred solution of tert-butyl N-ethyl-N-[1-[6-fluoro-7-[(7-methoxyimidazo[1,2-a]pyrimidin-6-yl)carbamoyl]-2-methyl-indazol-4-yl]-4-piperidyl]carbamate (70 mg, 123.54 μmol) in DCM (2 mL) was added TFA (1 mL) at rt. The resulting mixture was stirred for 2 h. The resulting mixture was concentrated under reduced pressure. The crude product was purified by Prep-HPLC with the following conditions (SunFire Prep C18 OBD Column 19*150 mm, 5 Um 10 nm, mobile phase, MeCN in water (0.05% TFA), 40% to 50% gradient in 7 min; detector, UV 254 nm) to afford 4-[4-(ethylamino)-1-piperidyl]-6-fluoro-N-(7-methoxyimidazo[1,2-a]pyrimidin-6-yl)-2-methyl-indazole-7-carboxamide (23.3 mg, 49.95 μmol, 40.43% yield) as a solid. LCMS (ES, m/z): 467 [M+H]⁺. ¹H NMR (400 MHz, DMSO-d₆) δ 11.92 (s, 1H), 10.07 (s, 1H), 8.94 (s, 1H), 8.59 (d, J=6.5 Hz, 2H), 8.11 (d, J=2.2 Hz, 1H), 7.90 (d, J=2.2 Hz, 1H), 6.36 (d, J=16.0 Hz, 1H), 4.29 (d, J=12.3 Hz, 6H), 4.13 (d, J=13.4 Hz, 2H), 3.15 (t, J=12.6 Hz, 2H), 3.08-3.00 (m, 2H), 2.18-2.10 (m, 2H), 1.73-1.60 (m, 2H), 1.23 (t, J=7.2 Hz, 3H).

### Example 227: Synthesis of Compound 694

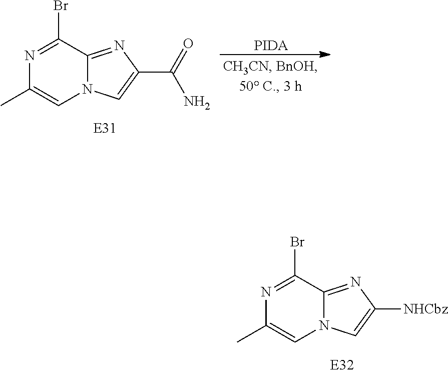 [SMILES: Cc1cn2cc(C(N)=O)nc2c(Br)n1 ; "PIDA" ; Cc1cn2cc(NC(=O)OCc3ccccc3)nc2c(Br)n1]

To a stirred solution of 8-bromo-6-methyl-imidazo[1,2-a]pyrazine-2-carboxamide (500 mg, 1.96 mmol) in BnOH (3.5 mL) and CH₃CN (7 mL) was added PIDA (1.89 g, 5.88 mmol) dropwise at 0° C. under N₂ atmosphere. The resulting mixture was stirred for 3 h at 50° C. The resulting mixture was diluted with water (5 mL). The resulting mixture purified by Prep-HPLC with the following conditions (column, C18 silica gel, XBridge, 19×150 mm; mobile phase, MeCN in water (0.05% NH₃·H₂O), 30% to 85% gradient in 21 min; detector, UV 254 nm) to afford benzyl N-(8-bromo-6-methyl-imidazo[1,2-a]pyrazin-2-yl)carbamate (260 mg, 719.84 μmol, 37% yield) as a solid. LCMS (ES, m/z): 361 [M+H]⁺.

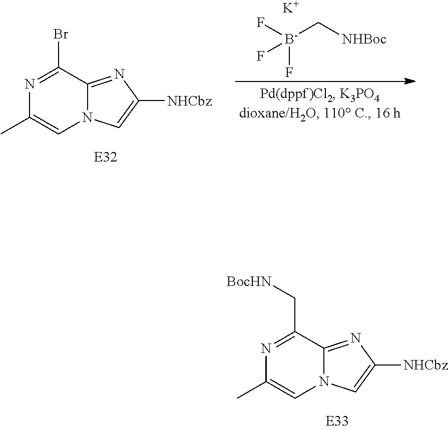 [SMILES: [K+] ; Cc1cn2cc(NC(=O)OCc3ccccc3)nc2c(Br)n1 ; CC(C)(C)OC(=O)NC[B-](F)(F)F ; Cc1cn2cc(NC(=O)OCc3ccccc3)nc2c(CNC(=O)OC(C)(C)C)n1]

To a stirred mixture of benzyl N-(8-bromo-6-methyl-imidazo[1,2-a]pyrazin-2-yl)carbamate (210 mg, 581.41 μmol) and (tert-butoxycarbonylamino)methyl-trifluoro-boranuide potassium (206.7 mg, 872.11 μmol) in dioxane (4 mL) and H₂O (0.4 mL) were added K₃PO₄ (246.8 mg, 1.16 mmol) and Pd(dppf)Cl₂ (47.4 mg, 58.14 μmol) at room temperature under nitrogen atmosphere. The resulting mixture was stirred for 16 h at 110° C. under nitrogen atmosphere. The resulting mixture was concentrated under reduced pressure. The residue was purified by silica gel column chromatography, eluted with PE/EA (2:1) to afford tert-butyl N-[[2-(benzyloxycarbonylamino)-6-methyl-imidazo[1,2-a]pyrazin-8-yl]methyl]carbamate (120 mg, 291.65 μmol, 50% yield) as a solid. LCMS (ES, m/z): 412 [M+H]⁺.

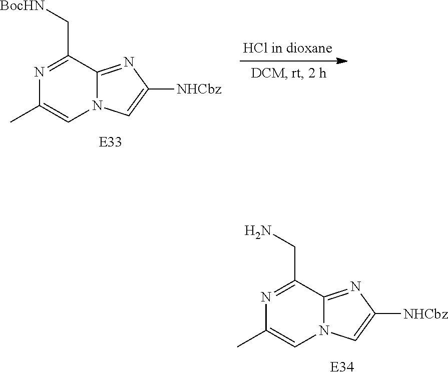 [SMILES: Cc1cn2cc(NC(=O)OCc3ccccc3)nc2c(CNC(=O)OC(C)(C)C)n1 ; Cc1cn2cc(NC(=O)OCc3ccccc3)nc2c(CN)n1]

A solution of tert-butyl N-[[2-(benzyloxycarbonylamino)-6-methyl-imidazo[1,2-a]pyrazin-8-yl]methyl]carbamate (120 mg, 291.65 μmol) in DCM (1 mL) was treated with HCl (4.0 M in 1,4-dioxane) (1 mL) at room temperature. The resulting mixture was stirred for 2 h at room temperature under nitrogen atmosphere. The resulting mixture was concentrated under reduced pressure to afford benzyl (8-(aminomethyl)-6-methylimidazo[1,2-a]pyrazin-2-yl)carbamate hydrochloride (86 mg, 247.27 μmol, 85% yield) as a solid. LCMS (ES, m/z): 312 [M+H]⁺.

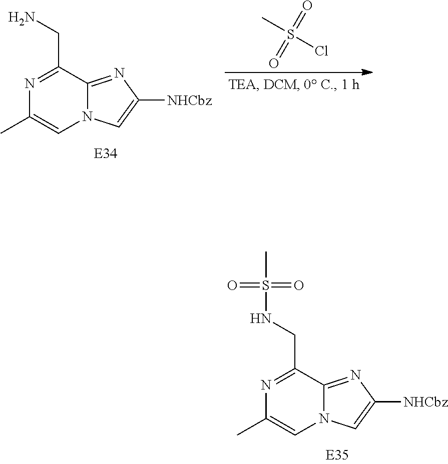

To a stirred solution of benzyl (8-(aminomethyl)-6-methylimidazo[1,2-a]pyrazin-2-yl)carbamate hydrochloride (86 mg, 276.23 μmol) and TEA (83.8 mg, 828.68 μmol) in DCM (2 mL) was added methanesulfonyl chloride (34.8 mg, 303.85 μmol) dropwise at 0° C. under N₂ atmosphere. The resulting mixture was stirred for 1 h at 0° C. The resulting mixture was diluted with water (10 mL). The resulting mixture was extracted with EA (3×10 mL). The combined organic layers were washed with water (10 mL) and brine (1×10 mL), dried over anhydrous Na₂SO₄. After filtration, the filtrate was concentrated under reduced pressure. The residue was purified by silica gel column chromatography, eluted with PE/EA (2:1) to afford benzyl N-[8-(methanesulfonamidomethyl)-6-methyl-imidazo[1,2-a]pyrazin-2-yl]carbamate (50 mg, 128.39 μmol, 46% yield) as a solid. LCMS (ES, m/z): 390 [M+H]⁺.

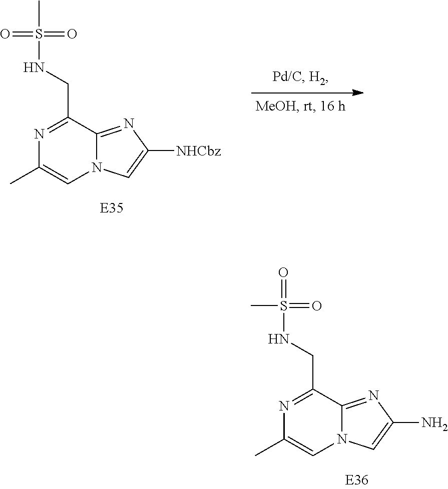 [SMILES: Cc1cn2cc(NC(=O)OCc3ccccc3)nc2c(CNS(C)(=O)=O)n1 ; Cc1cn2cc(N)nc2c(CNS(C)(=O)=O)n1]

To a solution of benzyl N-[8-(methanesulfonamidomethyl)-6-methyl-imidazo[1,2-a]pyrazin-2-yl]carbamate (50 mg, 128.39 μmol) in MeOH (4 mL) was added Pd/C (20 mg) under nitrogen atmosphere in a 50 mL round-bottom flask. The mixture was hydrogenated at room temperature for 16 h under hydrogen atmosphere using a hydrogen balloon, filtered through a Celite pad and concentrated under reduced pressure. This resulted in N-[(2-amino-6-methyl-imidazo[1,2-a]pyrazin-8-yl)methyl]methanesulfonamide (28 mg, 109.68 μmol, 85% yield) as a solid. LCMS (ES, m/z): 256 [M+H]⁺.

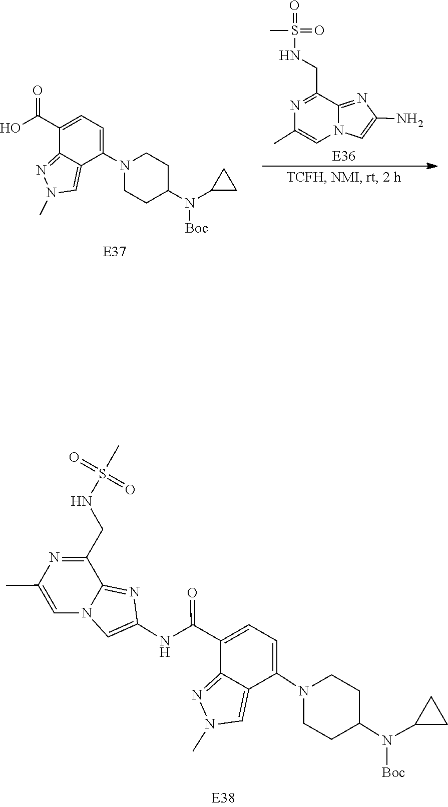

To a stirred mixture of N-[(2-amino-6-methyl-imidazo[1,2-a]pyrazin-8-yl)methyl]methanesulfonamide (22.1 mg, 86.85 μmol) and 4-[4-[tert-butoxycarbonyl(cyclopropyl)amino]-1-piperidyl]-2-methyl-indazole-7-carboxylic acid (30 mg, 72.38 μmol) in CH₃CN (1 mL) were added NMI (23.7 mg, 289.51 μmol) and TCFH (30.4 mg, 108.57 μmol) at room temperature. The resulting mixture was stirred for 2 h at rt. The resulting mixture was diluted with water (10 mL). The resulting mixture was extracted with EA (3×15 mL). The combined organic layers were washed with brine (1×10 mL), dried over anhydrous Na₂SO₄. After filtration, the filtrate was concentrated under reduced pressure. The residue was purified by silica gel column chromatography, eluted with EA to afford tert-butyl N-cyclopropyl-N-[1-[7-[[8-(methanesulfonamidomethyl)-6-methyl-imidazo[1,2-a]pyrazin-2-yl]carbamoyl]-2-methyl-indazol-4-yl]-4-piperidyl]carbamate (15 mg, 23.01 μmol, 32% yield) as a solid. LCMS (ES, m/z): 652 [M+H]⁺.

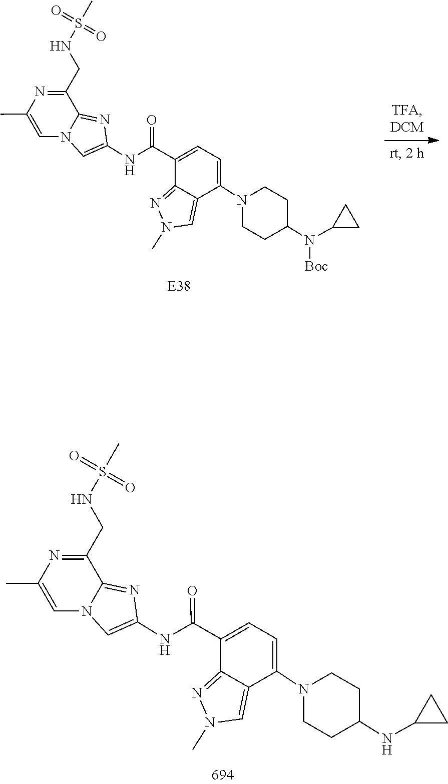 [SMILES: Cc1cn2cc(NC(=O)c3ccc(N4CCC(N(C(=O)OC(C)(C)C)C5CC5)CC4)c4cn(C)nc34)nc2c(CNS(C)(=O)=O)n1 ; Cc1cn2cc(NC(=O)c3ccc(N4CCC(NC5CC5)CC4)c4cn(C)nc34)nc2c(CNS(C)(=O)=O)n1]

A solution of tert-butyl N-cyclopropyl-N-[1-[7-[[8-(methanesulfonamidomethyl)-6-methyl-imidazo[1,2-a]pyrazin-2-yl]carbamoyl]-2-methyl-indazol-4-yl]-4-piperidyl]carbamate (15 mg, 23.01 μmol) in DCM (0.6 mL) was treated with TFA (0.2 mL) at room temperature. The resulting mixture was stirred for 2 h at room temperature under nitrogen atmosphere. The resulting mixture was concentrated under reduced pressure. The crude product was purified by Prep-HPLC with the following conditions (SunFire Prep C18 OBD Column 19*150 mm, 5 μm 10 nm, mobile phase, MeCN in water (0.05% TFA), 20% to 40% gradient in 25 min; detector, UV 254 nm) to afford 4-(4-(cyclopropylamino)piperidin-1-yl)-2-methyl-N-(6-methyl-8-(methylsulfonamidomethyl)imidazo[1,2-a]pyrazin-2-yl)-2H-indazole-7-carboxamide 2,2,2-trifluoroacetate (4.6 mg, 6.91 μmol, 30% yield) as a solid. LCMS (ES, m/z): 552 [M+H]⁺. ¹H NMR (400 MHz, Methanol-d₄) δ 8.54 (s, 1H), 8.35 (s, 1H), 8.24 (s, 1H), 8.10 (d, J=8.1 Hz, 1H), 6.59 (d, J=8.1 Hz, 1H), 4.76 (s, 2H), 4.38 (s, 3H), 4.14 (d, J=13.1 Hz, 2H), 3.62-3.50 (m, 1H), 3.13 (t, J=13.2 Hz, 2H), 3.08 (s, 3H), 2.87 (tt, J=7.4, 3.9 Hz, 1H), 2.50 (s, 3H), 2.43-2.30 (m, 2H), 1.91 (tt, J=12.8, 6.4 Hz, 2H), 1.06-0.97 (m, 2H), 0.93 (dt, J=5.7, 3.7 Hz, 2H).

### Example 228: Synthesis of Compound 695

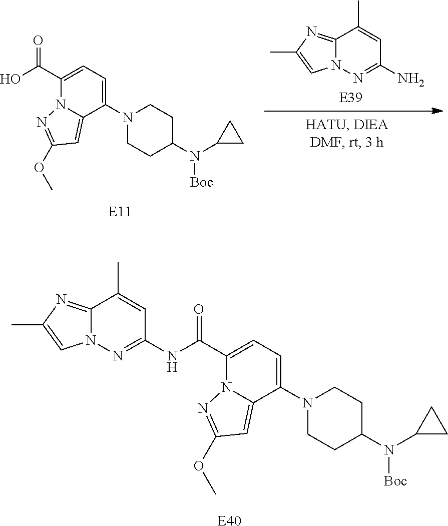

To a stirred mixture of 4-[4-[tert-butoxycarbonyl(cyclopropyl)amino]-1-piperidyl]-2-methoxy-pyrazolo[1,5-a]pyridine-7-carboxylic acid (0.05 g, 116.15 μmol) and 2,8-dimethylimidazo[1,2-b]pyridazin-6-amine (22.6 mg, 139.37 μmol) in DMF (2 mL) were added DIEA (45.0 mg, 348.44 μmol), HATU (66.4 mg, 174.22 μmol) at room temperature. The resulting mixture was stirred for 3 h at room temperature. The resulting mixture was diluted with water (5 mL). The resulting mixture was extracted with EA (3×5 mL). The combined organic layers were washed with water (2×10 mL), brine (1×10 mL), dried over anhydrous Na₂SO₄. After filtration, the filtrate was concentrated under reduced pressure to afford tert-butyl N-cyclopropyl-N-[1-[7-[(2,8-dimethylimidazo[1,2-b]pyridazin-6-yl)carbamoyl]-2-methoxy-pyrazolo[1,5-a]pyridin-4-yl]-4-piperidyl]carbamate (0.06 g, 104.41 μmol, 90% yield) as a solid.

LCMS (ES, m/z): 575 [M+H]⁺.

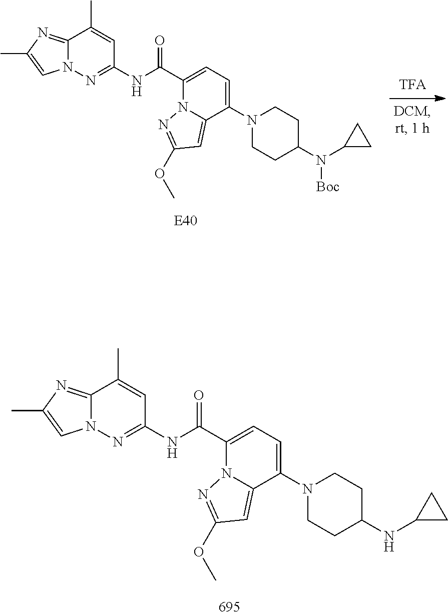

To a solution of tert-butyl N-cyclopropyl-N-[1-[7-[(2,8-dimethylimidazo[1,2-b]pyridazin-6-yl)carbamoyl]-2-methoxy-pyrazolo[1,5-a]pyridin-4-yl]-4-piperidyl]carbamate (0.065 g, 113.11 μmol) in DCM (1 mL) was added TFA (0.4 mL). The reaction was stirred for 1 h at rt. The resulting mixture was concentrated under reduced pressure and adjusted to pH 8 with saturated NaHCO₃ (aq.). After filtration, the filtrate was concentrated under reduced pressure. The residue was purified by reversed-phase flash chromatography with the following conditions (column, C18 silica gel; mobile phase, CH₃CN in water (0.1% NH₃·H₂O), 45% to 85% gradient in 10 min; detector, UV 254 to nm) afford 4-[4-(cyclopropylamino)-1-piperidyl]-N-(2,8-dimethylimidazo[1,2-b]pyridazin-6-yl)-2-methoxy-pyrazolo[1,5-a]pyridine-7-carboxamide (0.02 g, 42.14 μmol, 37% yield) as a solid. LCMS (ES, m/z): 475 [M+H]⁺. ¹H NMR (300 MHz, DMSO-d₆) δ 12.89 (s, 1H), 8.01 (s, 1H), 7.93 (s, 1H), 7.75 (d, J=8.2 Hz, 1H), 6.77 (d, J=8.3 Hz, 1H), 6.29 (s, 1H), 4.05 (s, 3H), 3.67 (d, J=12.5 Hz, 2H), 2.91 (t, J=11.5 Hz, 2H), 2.73 (s, 1H), 2.56 (s, 3H), 2.36 (s, 3H), 2.14-2.06 (m, 2H), 1.99 (d, J=12.8 Hz, 2H), 1.49 (q, J=10.7 Hz, 2H), 1.22 (s, 3H), 0.83 (d, J=7.0 Hz, 1H), 0.42-0.34 (m, 2H), 0.21 (dd, J=6.6, 3.7 Hz, 2H).

### Example 229: Synthesis of Compound 697

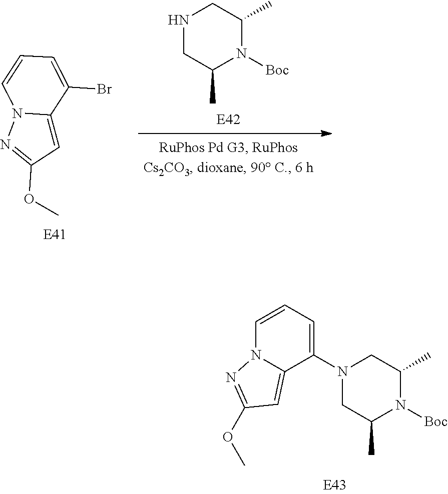 [SMILES: COc1cc2c(Br)cccn2n1 ; C[C@H]1CNC[C@H](C)N1C(=O)OC(C)(C)C ; COc1cc2c(N3C[C@H](C)N(C(=O)OC(C)(C)C)[C@@H](C)C3)cccn2n1]

To a stirred mixture of 4-bromo-2-methoxy-pyrazolo[1,5-a]pyridine (1 g, 4.40 mmol) and tert-butyl (2S,6S)-2,6-dimethylpiperazine-1-carboxylate (1.13 g, 5.29 mmol) in dioxane (20 mL) were added Cs₂CO₃ (4.30 g, 13.21 mmol) and RuPhos Pd G3 (368.4 mg, 440.42 μmol) and RuPhos (411.0 mg, 880.83 μmol) at room temperature under nitrogen atmosphere. The resulting mixture was stirred for 6 h at 90° C. under nitrogen atmosphere. The mixture was concentrated under reduced pressure. The residue was purified by silica gel column chromatography, eluted with PE/EA (2:1) to afford tert-butyl (2S,6S)-4-(2-methoxypyrazolo[1,5-a]pyridin-4-yl)-2,6-dimethyl-piperazine-1-carboxylate (1 g, 2.77 mmol, 63% yield) as a solid. LCMS (ES, m/z): 361 [M+H]⁺.

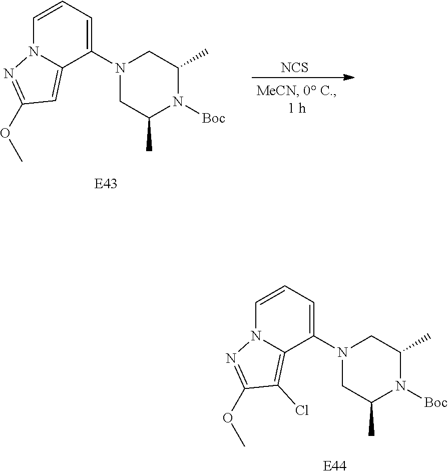 [SMILES: COc1cc2c(N3C[C@H](C)N(C(=O)OC(C)(C)C)[C@@H](C)C3)cccn2n1 ; "NCS" ; COc1nn2cccc(N3C[C@H](C)N(C(=O)OC(C)(C)C)[C@@H](C)C3)c2c1Cl]

To a stirred mixture of tert-butyl (2S,6S)-4-(2-methoxypyrazolo[1,5-a]pyridin-4-yl)-2,6-dimethyl-piperazine-1-carboxylate (1 g, 2.77 mmol) in MeCN (20 mL) was added NCS (370.5 mg, 2.77 mmol) at 0° C. The resulting mixture was stirred for 1 h at 0° C. The resulting mixture was diluted with water (20 mL). The precipitated solids were collected by filtration and washed with water (3×30 mL). The solid was dried to afford tert-butyl (2S,6S)-4-(3-chloro-2-methoxy-pyrazolo[1,5-a]pyridin-4-yl)-2,6-dimethyl-piperazine-1-carboxylate (860 mg, 2.18 mmol, 79% yield) as a solid. LCMS (ES, m/z): 395 [M+H]⁺.

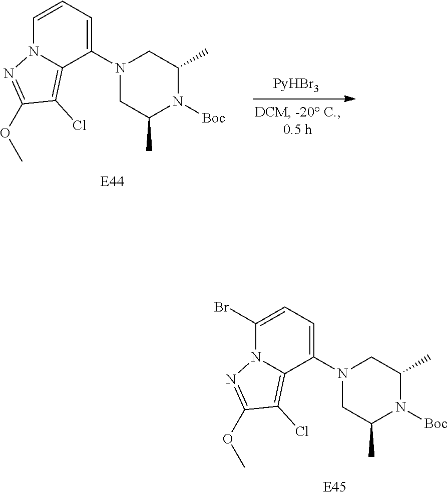 [SMILES: COc1nn2cccc(N3C[C@H](C)N(C(=O)OC(C)(C)C)[C@@H](C)C3)c2c1Cl ; "PyHBr3" ; COc1nn2c(Br)ccc(N3C[C@H](C)N(C(=O)OC(C)(C)C)[C@@H](C)C3)c2c1Cl]

To a stirred mixture of tert-butyl (2S,6S)-4-(3-chloro-2-methoxy-pyrazolo[1,5-a]pyridin-4-yl)-2,6-dimethyl-piperazine-1-carboxylate (780 mg, 1.98 mmol) in DCM (16 mL) was added PyHBr₃ (631.7 mg, 1.98 mmol) at 0° C. The resulting mixture was stirred for 0.5 h at −20° C. The reaction was quenched with water at 0° C. The resulting mixture was diluted with water (20 mL). The resulting mixture was extracted with DCM (3×20 mL). The combined organic layers were washed with water (3×50 mL) and brine (1×50 mL), dried over anhydrous Na₂SO₄. After filtration, the filtrate was concentrated under reduced pressure. The residue was purified by silica gel column chromatography, eluted with PE/EA (1:1) to afford tert-butyl (2S,6S)-4-(7-bromo-3-chloro-2-methoxy-pyrazolo[1,5-a]pyridin-4-yl)-2,6-dimethyl-piperazine-1-carboxylate (560 mg, 1.18 mmol, 60% yield) as a solid. LCMS (ES, m/z): 473 [M+H]⁺.

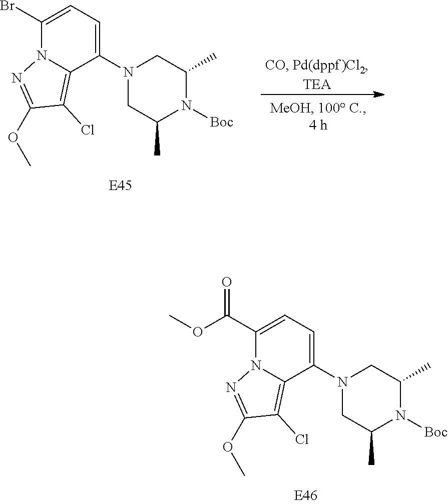 [SMILES: COc1nn2c(Br)ccc(N3C[C@H](C)N(C(=O)OC(C)(C)C)[C@@H](C)C3)c2c1Cl ; "CO, Pd(dppf)Cl2, TEA" ; COC(=O)c1ccc(N2C[C@H](C)N(C(=O)OC(C)(C)C)[C@@H](C)C2)c2c(Cl)c(OC)nn12]

To a solution of tert-butyl (2S,6S)-4-(7-bromo-3-chloro-2-methoxy-pyrazolo[1,5-a]pyridin-4-yl)-2,6-dimethyl-piperazine-1-carboxylate (560 mg, 1.18 mmol) in MeOH (10 mL) were added Pd(dppf)Cl₂ (864.8 mg, 1.18 mmol) and TEA (119.6 mg, 1.18 mmol) in a pressure tank. The mixture was purged with nitrogen for 3 min and then was pressurized to 1 MPa with carbon monoxide at 100° C. for 4 h. The reaction mixture was cooled to room temperature and filtered to remove insoluble solids. The resulting mixture was concentrated under reduced pressure. The residue was purified by silica gel column chromatography, eluted with EA to afford methyl 4-[(3S,5S)-4-tert-butoxycarbonyl-3,5-dimethyl-piperazin-1-yl]-3-chloro-2-methoxy-pyrazolo[1,5-a]pyridine-7-carboxylate (450 mg, 993.53 μmol, 84% yield) as a solid.

LCMS (ES, m/z): 453 [M+H]⁺.

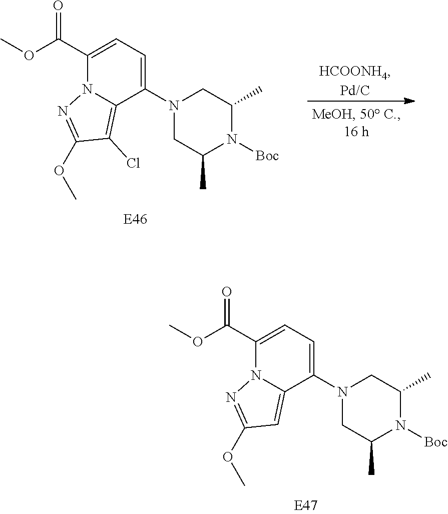 [SMILES: COC(=O)c1ccc(N2C[C@H](C)N(C(=O)OC(C)(C)C)[C@@H](C)C2)c2c(Cl)c(OC)nn12 ; "HCOONH4,Pd/C" ; COC(=O)c1ccc(N2C[C@H](C)N(C(=O)OC(C)(C)C)[C@@H](C)C2)c2cc(OC)nn12]

To a stirred mixture of methyl 4-[(3S,5S)-4-tert-butoxycarbonyl-3,5-dimethyl-piperazin-1-yl]-3-chloro-2-methoxy-pyrazolo[1,5-a]pyridine-7-carboxylate (450 mg, 993.53 μmol) in MeOH (9 mL) were added COOHNH₄ (125.2 mg, 1.99 mmol) and Pd/C (20% on Carbon (wetted with ca. 55% water)) (45 mg) at room temperature under nitrogen atmosphere. The resulting mixture was stirred for 16 h at 50° C. under nitrogen atmosphere, filtered through a celite pad and concentrated under reduced pressure. The residue was purified by silica gel column chromatography, eluted with EA to afford methyl 4-[(3S,5S)-4-tert-butoxycarbonyl-3,5-dimethyl-piperazin-1-yl]-2-methoxy-pyrazolo[1,5-a]pyridine-7-carboxylate (220 mg, 525.71 μmol, 53% yield) as a solid. LCMS (ES, m/z): 419 [M+H]⁺.

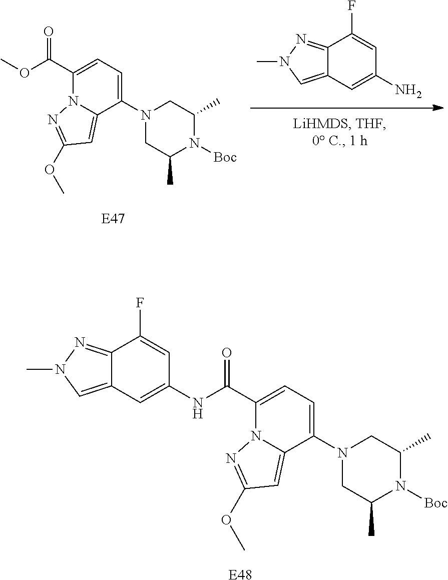

To a stirred mixture of methyl 4-[(3S,5S)-4-tert-butoxycarbonyl-3,5-dimethyl-piperazin-1-yl]-2-methoxy-pyrazolo[1,5-a]pyridine-7-carboxylate (60 mg, 143.37 μmol) and 7-fluoro-2-methyl-indazol-5-amine (28.4 mg, 172.05 μmol) in THF (1.2 mL) was added LiHMDS (24.0 mg, 143.37 μmol) at 0° C. The resulting mixture was stirred for 1 h at 0° C. The reaction was quenched with water at 0° C. The resulting mixture was extracted with EA (3×2 mL). The combined organic layers were washed with water (3×5 mL) and brine (1×5 mL), dried over anhydrous Na₂SO₄. After filtration, the filtrate was concentrated under reduced pressure. The residue was purified by silica gel column chromatography, eluted with DCM/MeOH (10:1) to afford tert-butyl (2S,6S)-4-[7-[(7-fluoro-2-methyl-indazol-5-yl)carbamoyl]-2-hydroxy-pyrazolo[1,5-a]pyridin-4-yl]-2,6-dimethyl-piperazine-1-carboxylate (75 mg, 139.51 μmol, 97% yield) as a solid. LCMS (ES, m/z): 552 [M+H]⁺.

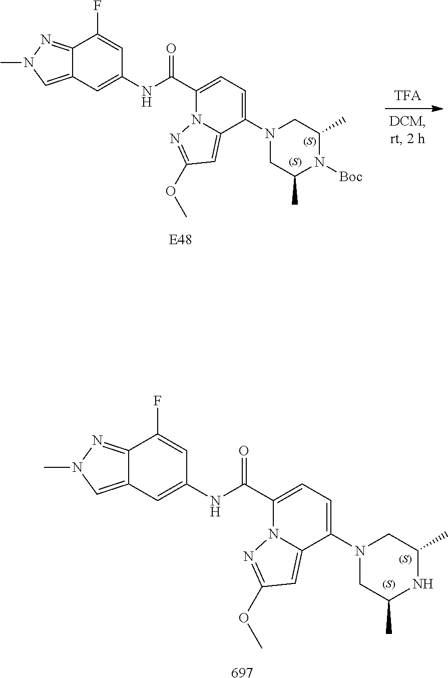

To a stirred mixture of tert-butyl (2S,6S)-4-[7-[(7-fluoro-2-methyl-indazol-5-yl)carbamoyl]-2-methoxy-pyrazolo[1,5-a]pyridin-4-yl]-2,6-dimethyl-piperazine-1-carboxylate (75 mg, 135.97 μmol) in DCM (1 mL) was added TFA (0.25 mL) dropwise at room temperature. The resulting mixture was stirred for 2 h at room temperature. The resulting mixture were concentrated under reduced pressure. The crude product was purified by Prep-HPLC with the following conditions (column, C18 silica gel, XBridge, 19×150 mm; mobile phase, MeCN in water (0.05% NH₃·H₂O), 40% to 90% gradient in 7 min; detector, UV 254 nm) to afford 4-[(3S,5S)-3,5-dimethylpiperazin-1-yl]-N-(7-fluoro-2-methyl-indazol-5-yl)-2-methoxy-pyrazolo[1,5-a]pyridine-7-carboxamide (20 mg, 44.30 μmol, 33% yield) as a solid. LCMS (ES, m/z): 452 [M+H]-1H NMR (400 MHz, DMSO-d₆) δ 12.26 (s, 1H), 8.46 (s, 1H), 8.07 (s, 1H), 7.68 (d, J=8.1 Hz, 1H), 7.36 (d, J=13.1 Hz, 1H), 6.76 (d, J=8.1 Hz, 1H), 6.14 (s, 1H), 4.20 (s, 3H), 4.11 (s, 3H), 3.26 (s, 2H), 3.17 (d, J=11.4 Hz, 2H), 2.94 (dd, J=11.6, 6.2 Hz, 2H), 1.17 (d, J=6.4 Hz, 6H).

### Example 230: Synthesis of Compound 698

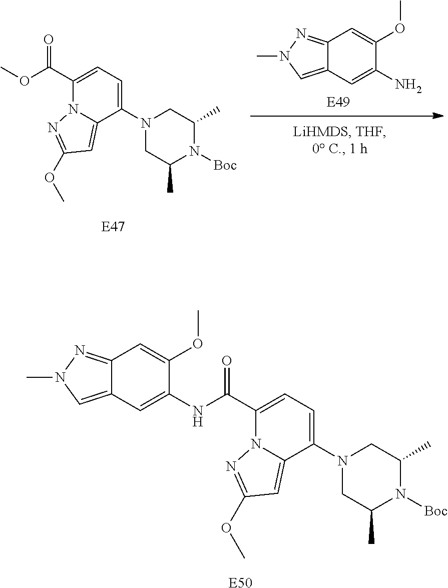

To a stirred mixture of methyl 4-[(3S,5S)-4-tert-butoxycarbonyl-3,5-dimethyl-piperazin-1-yl]-2-methoxy-pyrazolo[1,5-a]pyridine-7-carboxylate (60 mg, 143.37 μmol) and 6-methoxy-2-methyl-indazol-5-amine (28.0 mg, 157.71 μmol) in THF (1.5 mL) was added LiHMDS (0.57 mL, 570.0 μmol, 1 mol/L in THF) at 0° C. The resulting mixture was stirred for 1 h at 0° C. The reaction was quenched with water at 0° C. The resulting mixture was diluted with water (2 mL). The resulting mixture was extracted with EA (3×2 mL). The combined organic layers were washed with water (3×5 mL) and brine (1×5 mL), dried over anhydrous Na₂SO₄. After filtration, the filtrate was concentrated under reduced pressure. The residue was purified by silica gel column chromatography, eluted with DCM/MeOH (10:1) to afford tert-butyl (2S,6S)-4-[2-methoxy-7-[(6-methoxy-2-methyl-indazol-5-yl)carbamoyl]pyrazolo[1,5-a]pyridin-4-yl]-2,6-dimethyl-piperazine-1-carboxylate (80 mg, 141.93 μmol, 99% yield) as a solid. LCMS (ES, m/z): 564 [M+H]⁺.

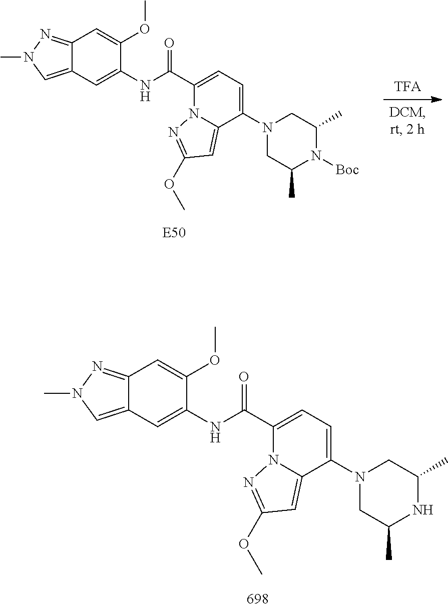

To a stirred mixture of tert-butyl (2S,6S)-4-[2-methoxy-7-[(6-methoxy-2-methyl-indazol-5-yl)carbamoyl]pyrazolo[1,5-a]pyridin-4-yl]-2,6-dimethyl-piperazine-1-carboxylate (80 mg, 141.93 μmol) in DCM (2 mL) was added TFA (0.5 mL) dropwise at room temperature. The resulting mixture was stirred for 2 h at room temperature. The resulting mixture was concentrated under reduced pressure. The crude product was purified by Prep-HPLC with the following condition (column, C18 silica gel, XBridge, 19×150 mm; mobile phase, MeCN in water (0.05% NH₃·H₂O), 40% to 90% gradient in 7 min; detector, UV 254 nm) to afford 4-[(3S,5S)-3,5-dimethylpiperazin-1-yl]-2-methoxy-N-(6-methoxy-2-methyl-indazol-5-yl) pyrazolo[1,5-a]pyridine-7-carboxamide (24 mg, 51.78 μmol, 36% yield) as a solid. LCMS (ES, m/z): 464 [M+H]⁺. ¹H NMR (400 MHz, DMSO-d₆) δ 12.51 (s, 1H), 8.82 (s, 1H), 8.24 (s, 1H), 7.77 (d, J=8.2 Hz, 1H), 7.12 (s, 1H), 6.77 (d, J=8.2 Hz, 1H), 6.14 (s, 1H), 4.11 (s, 3H), 4.09 (s, 3H), 3.96 (s, 3H), 3.26 (s, 2H), 3.17 (d, J=11.6 Hz, 2H), 2.94 (dd, J=11.6, 6.1 Hz, 2H), 1.17 (d, J=6.4 Hz, 6H).

### Example 231: Synthesis of Compound 699

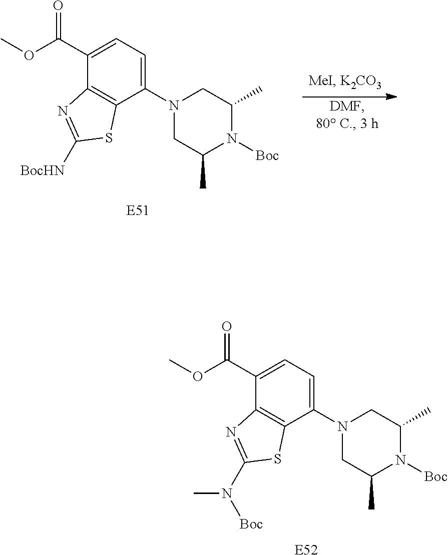 [SMILES: COC(=O)c1ccc(N2C[C@H](C)N(C(=O)OC(C)(C)C)[C@@H](C)C2)c2sc(NC(=O)OC(C)(C)C)nc12 ; "DMF, 80° C., 3 h" ; "E51" ; COC(=O)c1ccc(N2C[C@H](C)N(C(=O)OC(C)(C)C)[C@@H](C)C2)c2sc(N(C)C(=O)OC(C)(C)C)nc12 ; "E52"]

To a solution of methyl 2-(tert-butoxycarbonylamino)-7-[(3S,5S)-4-tert-butoxycarbonyl-3,5-dimethyl-piperazin-1-yl]-1,3-benzothiazole-4-carboxylate (130 mg, 249.69 μmol) in DMF (1.5 mL) were added K₂CO₃ (103.5 mg, 749.08 μmol) and iodomethane (70.9 mg, 499.39 μmol). The mixture was stirred for 3 h at 80° C. The reaction was diluted with H₂O (10 mL). The resulting mixture was extracted with EA (2×15 mL). The combined organic layers were washed with H₂O (2×15 mL), dried over anhydrous Na₂SO₄. After filtration, the filtrate was concentrated under reduced pressure. The residue was purified by silica gel column chromatography, eluted with PE/EA (3:1) to afford methyl 7-[(3S,5S)-4-tert-butoxycarbonyl-3,5-dimethyl-piperazin-1-yl]-2-[tert-butoxycarbonyl(methyl)amino]-1,3-benzothiazole-4-carboxylate (130 mg, 243.14 μmol, 97% yield) as a solid. LCMS (ES, m/z): 535 [M+H]⁺.

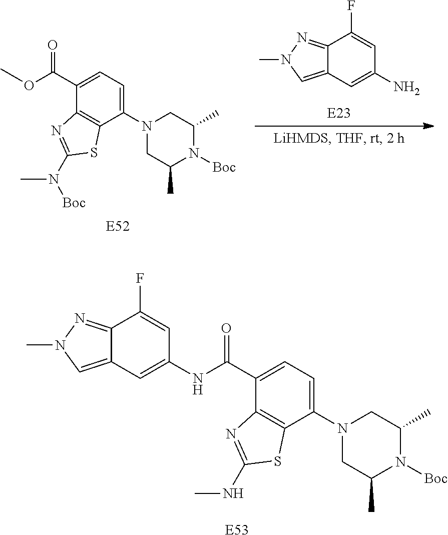

To a solution of methyl 7-[(3S,5S)-4-tert-butoxycarbonyl-3,5-dimethyl-piperazin-1-yl]-2-[tert-butoxycarbonyl(methyl)amino]-1,3-benzothiazole-4-carboxylate (60 mg, 112.22 μmol) in THF (0.7 mL) was added 7-fluoro-2-methyl-indazol-5-amine (24.1 mg, 145.89 μmol) at rt. Then LiHMDS (448.9 μL, 448.9 μmol) (1 mol/L in THF) was added dropwise at 0° C. The mixture was stirred for 2 h at rt. The reaction was diluted with H₂O (10 mL). The resulting mixture was extracted with EA (2×10 mL). The combined organic layers were washed with H₂O (10 mL), dried over anhydrous Na₂SO₄. After filtration, the filtrate was concentrated under reduced pressure. The residue was purified by silica gel column chromatography, eluted with DCM/EA (3:1) to afford tert-butyl (2S,6S)-4-[4-[(7-fluoro-2-methyl-indazol-5-yl)carbamoyl]-2-(methylamino)-1,3-benzothiazol-7-yl]-2,6-dimethyl-piperazine-1-carboxylate (60 mg, 105.69 μmol, 94% yield) as a solid. LCMS (ES, m/z): 568 [M+H]⁺.

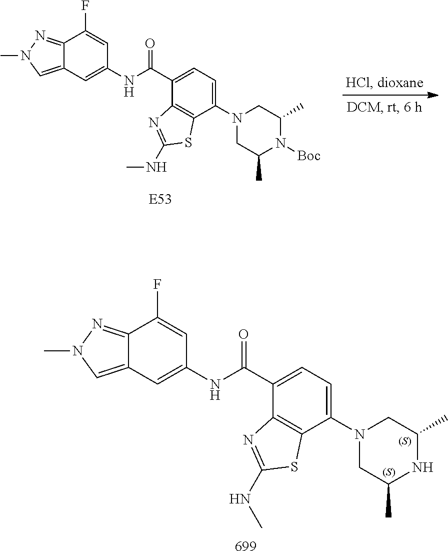

To a solution of tert-butyl (2S,6S)-4-[2-[tert-butoxycarbonyl(methyl)amino]-4-[(7-fluoro-2-methyl-indazol-5-yl)carbamoyl]-1,3-benzothiazol-7-yl]-2,6-dimethyl-piperazine-1-carboxylate (60 mg, 89.85 μmol) in DCM (0.7 mL) was added HCl (4.0 M in 1,4-dioxane) (243 μL) at rt. The mixture was stirred for 6 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 40% to 55% gradient in 7 min; detector, UV 254 nm) to afford 7-[(3S,5S)-3,5-dimethylpiperazin-1-yl]-N-(7-fluoro-2-methyl-indazol-5-yl)-2-(methylamino)-1,3-benzothiazole-4-carboxamide (25 mg, 53.47 μmol, 60% yield) as a solid. LCMS (ES, m/z): 468 [M+H]⁺. ¹H NMR (400 MHz, DMSO-d₆) δ 12.36 (s, 1H), 8.72 (s, 1H), 8.41 (d, J=2.8 Hz, 1H), 8.10 (s, 1H), 8.00 (d, J=8.5 Hz, 1H), 7.33 (d, J=13.1 Hz, 1H), 6.81 (d, J=8.6 Hz, 1H), 4.18 (s, 3H), 3.25-3.18 (m, 2H), 3.14 (d, J=4.1 Hz, 3H), 3.06 (dd, J=11.3, 3.2 Hz, 2H), 2.87 (dd, J=11.3, 6.1 Hz, 2H), 2.04 (s, 1H), 1.23 (s, 1H), 1.16 (d, J=6.4 Hz, 6H).

### Example 232: Synthesis of Compound 700

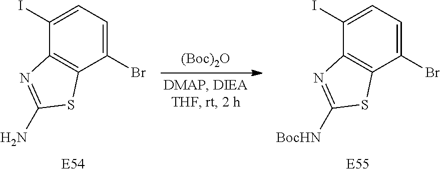 [SMILES: "(Boc)2O" ; Nc1nc2c(I)ccc(Br)c2s1 ; CC(C)(C)OC(=O)Nc1nc2c(I)ccc(Br)c2s1]

To a solution of 7-bromo-4-iodo-1,3-benzothiazol-2-amine (500 mg, 1.41 mmol) in THF (5 mL) were added DIEA (364.1 mg, 2.82 mmol), tert-butoxycarbonyl tert-butyl carbonate (368.8 mg, 1.69 mmol) and DMAP (3.4 mg, 28.17 μmol). The mixture was stirred for 2 h at rt. The reaction was diluted with H₂O (20 mL). The resulting mixture was extracted with EA (2×25 mL). The combined organic layers were washed with H₂O (2×20 mL), dried over anhydrous Na₂SO₄. After filtration, the filtrate was concentrated under reduced pressure. The residue was purified by silica gel column chromatography, eluted with PE/EA (15/1) to afford tert-butyl (7-bromo-4-iodobenzo[d]thiazol-2-yl)carbamate (540 mg, 1.19 mmol, 84% yield) as a solid. LCMS (ES, m/z): 455 [M+H]⁺.

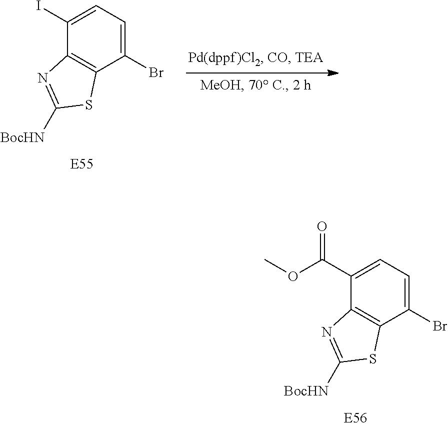 [SMILES: CC(C)(C)OC(=O)Nc1nc2c(I)ccc(Br)c2s1 ; COC(=O)c1ccc(Br)c2sc(NC(=O)OC(C)(C)C)nc12]

To a solution of tert-butyl N-(7-bromo-4-iodo-1,3-benzothiazol-2-yl)carbamate (670 mg, 1.47 mmol) in MeOH (20 mL) were added TEA (744.8 mg, 7.36 mmol) and Pd(dppf)Cl₂ (120.2 mg, 147.22 μmol) in a pressure tank. The mixture was pressurized to 20 atm with carbon monoxide at 70° C. for 2 h. The reaction mixture was cooled to room temperature and filtered to remove insoluble solids. The resulting mixture was concentrated under reduced pressure. The residue was purified by silica gel column chromatography, eluted with PE/DCM (7/3) to afford methyl 7-bromo-2-(tert-butoxycarbonylamino)-1,3-benzothiazole-4-carboxylate (330 mg, 852.17 μmol, 58% yield) as a solid. LCMS (ES, m/z): 387 [M+H]⁺.

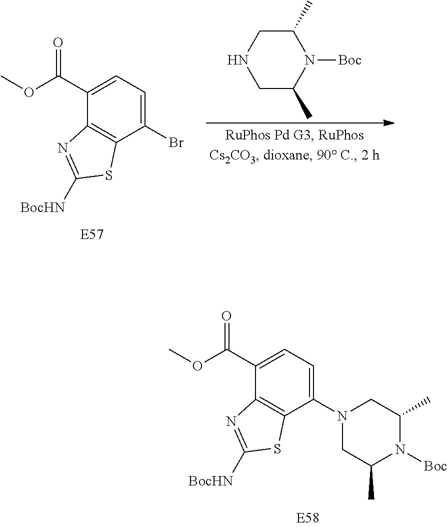 [SMILES: COC(=O)c1ccc(Br)c2sc(NC(=O)OC(C)(C)C)nc12 ; C[C@H]1CNC[C@H](C)N1C(=O)OC(C)(C)C ; COC(=O)c1ccc(N2C[C@H](C)N(C(=O)OC(C)(C)C)[C@@H](C)C2)c2sc(NC(=O)OC(C)(C)C)nc12]

To a stirred mixture of methyl 7-bromo-2-(tert-butoxycarbonylamino)-1,3-benzothiazole-4-carboxylate (310 mg, 800.52 μmol) in dioxane (4 mL) were added tert-butyl (2S,6S)-2,6-dimethylpiperazine-1-carboxylate (205.8 mg, 960.62 μmol), Cs₂CO₃ (521.6 mg, 1.60 mmol), Ruphos (74.7 mg, 160.10 μmol) and RuPhos Pd G3 (66.9 mg, 80.05 μmol) at rt under N₂. The mixture was stirred for 2 h at 90° C. The reaction was diluted with H₂O (20 mL). The resulting mixture was extracted with DCM (2×20 mL). The combined organic layers were washed with H₂O (20 mL), dried over anhydrous Na₂SO₄. After filtration, the filtrate was concentrated under reduced pressure. The residue was purified by silica gel column chromatography, eluted with PE/EA (3/1) to afford methyl 2-(tert-butoxycarbonylamino)-7-[(3S,5S)-4-tert-butoxycarbonyl-3,5-dimethyl-piperazin-1-yl]-1,3-benzothiazole-4-carboxylate (310 mg, 595.42 μmol, 74% yield) as +a solid. LCMS (ES, m/z): 521 [M+H]⁺.

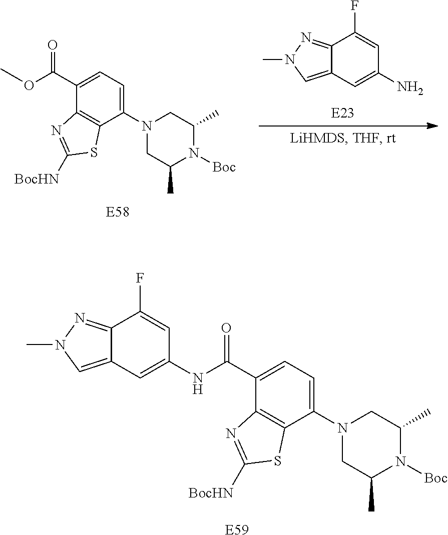

To a solution of methyl 2-(tert-butoxycarbonylamino)-7-[(3S,5S)-4-tert-butoxycarbonyl-3,5-dimethyl-piperazin-1-yl]-1,3-benzothiazole-4-carboxylate (120 mg, 230.49 μmol) in THF (1.5 mL) was added 7-fluoro-2-methyl-indazol-5-amine (49.5 mg, 299.63 μmol) at rt. Then LiHMDS (920 μL, 1 mol/L in THF) was added dropwise at 0° C. under N₂. The mixture was stirred for 2 h at rt. The reaction was quenched with NH₄Cl (aq.). The resulting mixture was extracted with DCM (2×15 mL). The combined organic layers were washed with H₂O (10 mL), dried over anhydrous Na₂SO₄. After filtration, the filtrate was concentrated under reduced pressure. The residue was purified by silica gel column chromatography, eluted with DCM/EA (3/7) to afford tert-butyl (2S,6S)-4-[2-(tert-butoxycarbonylamino)-4-[(7-fluoro-2-methyl-indazol-5-yl)carbamoyl]-1,3-benzothiazol-7-yl]-2,6-dimethyl-piperazine-1-carboxylate (150 mg, 229.44 μmol, 99% yield) as a solid. LCMS (ES, m/z): 654 [M+H]⁺.

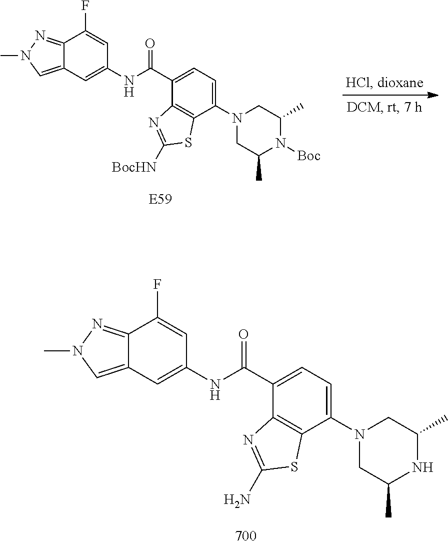

To a solution of tert-butyl (2S,6S)-4-[2-(tert-butoxycarbonylamino)-4-[(7-fluoro-2-methyl-indazol-5-yl)carbamoyl]-1,3-benzothiazol-7-yl]-2,6-dimethyl-piperazine-1-carboxylate (70 mg, 107.07 μmol) in DCM (1 mL) was added HCl (4.0 M in 1,4-dioxane) (250 μL) at rt. The mixture was stirred for 7 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃·H₂O), 25% to 55% gradient in 7 min; detector, UV 254 nm) to afford 2-amino-7-[(3S,5S)-3,5-dimethylpiperazin-1-yl]-N-(7-fluoro-2-methyl-indazol-5-yl)-1,3-benzothiazole-4-carboxamide (25 mg, 55.12 μmol, 51% yield) as a solid. LCMS (ES, m/z): 454 [M+H]⁺. ¹H NMR (400 MHz, DMSO-d₆) δ 12.43 (s, 1H), 8.42 (d, J=2.8 Hz, 1H), 8.27 (s, 2H), 8.23 (d, J=1.6 Hz, 1H), 7.98 (d, J=8.5 Hz, 1H), 7.48 (dd, J=13.2, 1.6 Hz, 1H), 6.79 (d, J=8.6 Hz, 1H), 4.19 (s, 3H), 3.21 (td, J=6.2, 3.1 Hz, 2H), 3.05 (dd, J=11.2, 3.1 Hz, 2H), 2.88 (dd, J=11.3, 6.1 Hz, 2H), 2.10 (s, 1H), 1.17 (d, J=6.4 Hz, 6H).

### Example 233: Synthesis of Compound 701

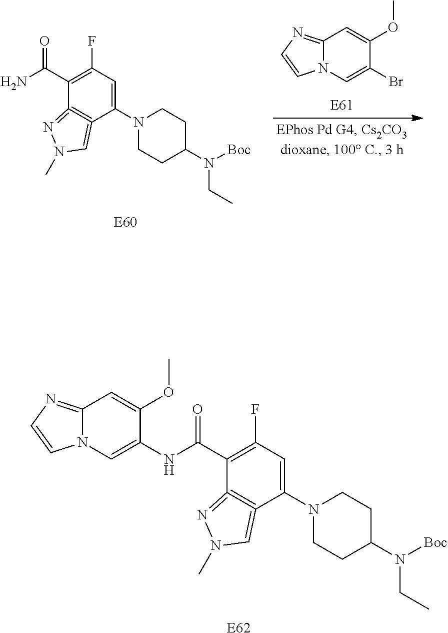

To a stirred mixture of tert-butyl N-[1-(7-carbamoyl-6-fluoro-2-methyl-indazol-4-yl)-4-piperidyl]-N-ethyl-carbamate (70 mg, 166.87 μmol) and 6-bromo-7-methoxy-imidazo[1,2-a]pyridine (45.4 mg, 200.24 μmol) in Dioxane (1.2 mL) were added Cs₂CO₃ (108.7 mg, 333.74 μmol) and EPhos Pd G3 (15.3 mg, 16.69 μmol) at room temperature under nitrogen atmosphere. The resulting mixture was stirred for 3 h at 100° C. under nitrogen atmosphere. The resulting mixture was concentrated under reduced pressure. The residue was purified by silica gel column chromatography, eluted with PE/EA (1:3) to afford tert-butyl N-ethyl-N-[1-[6-fluoro-7-[(7-methoxyimidazo[1,2-a]pyridin-6-yl)carbamoyl]-2-methyl-indazol-4-yl]-4-piperidyl]carbamate (80 mg, 141.43 μmol, 85% yield) as a solid. LCMS (ES, m/z): 566 [M+H]⁺.

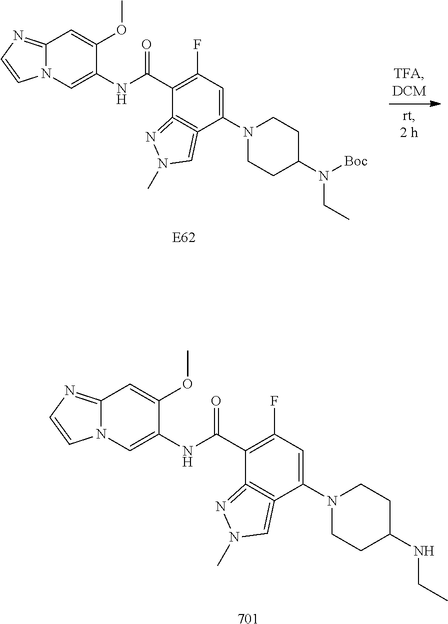

To a stirred solution of tert-butyl N-ethyl-N-[1-[6-fluoro-7-[(7-methoxyimidazo[1,2-a]pyridin-6-yl)carbamoyl]-2-methyl-indazol-4-yl]-4-piperidyl]carbamate (80 mg, 141.43 μmol) in DCM (2 mL) was added TFA (1 mL) at rt. The resulting mixture was stirred for 2 h. The resulting mixture was concentrated under reduced pressure. The crude product was purified by Prep-HPLC with the following conditions (SunFire Prep C18 OBD Column 19*150 mm, 5 Um 10 nm, mobile phase, CH₃CN in water (0.05% NH₄OH), 50% to 60% gradient in 7 min; detector, UV 254 nm) to afford 4-[4-(ethylamino)-1-piperidyl]-6-fluoro-N-(7-methoxyimidazo[1,2-a]pyridin-6-yl)-2-methyl-indazole-7-carboxamide (16.8 mg, 36.09 μmol, 26% yield) as a solid. LCMS (ES, m/z): 466 [M+H]⁺. ¹H NMR (400 MHz, DMSO-d₆) δ 11.65 (s, 1H), 9.65 (s, 1H), 8.82 (s, 1H), 7.84 (s, 1H), 7.37 (s, 1H), 7.09 (s, 1H), 6.23 (d, J=16.2 Hz, 1H), 4.24 (s, 3H), 4.08 (s, 3H), 3.93 (d, J=12.8 Hz, 2H), 3.13 (t, J=11.8 Hz, 2H), 2.71 (s, 1H), 2.61 (q, J=7.1 Hz, 2H), 1.95 (d, J=12.8 Hz, 2H), 1.44-1.38 (d, J=10.5 Hz, 1H), 1.04 (t, J=7.1 Hz, 3H).

### Example 234: Synthesis of Compound 703

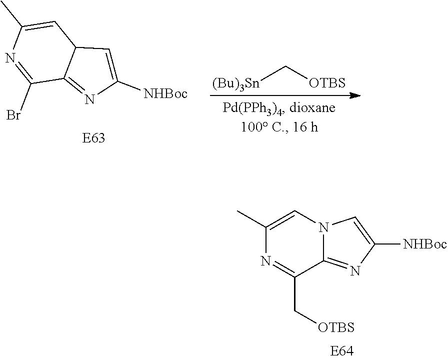

To a stirred mixture of tert-butyl N-(7-bromo-5-methyl-3aH-pyrrolo[2,3-c]pyridin-2-yl)carbamate (2 g, 6.13 mmol) and tert-butyl-dimethyl-(tributylstannylmethoxy)silane (5.34 g, 12.26 mmol) in Dioxane (40 mL) was added and Pd(PPh₃)₄ (1.42 g, 1.23 mmol) at room temperature under nitrogen atmosphere. The resulting mixture was stirred for 16 h at 100° C. under nitrogen atmosphere. The resulting mixture was concentrated under reduced pressure. The residue was purified by silica gel column chromatography, eluted with PE/EA (2:1) to afford tert-butyl N-[8-[[tert-butyl(dimethyl)silyl]oxymethyl]-6-methyl-imidazo[1,2-a]pyrazin-2-yl]carbamate (780 mg, 1.99 mmol, 32.41% yield) as a solid LCMS (ES, m/z): 393 [M+H]⁺.

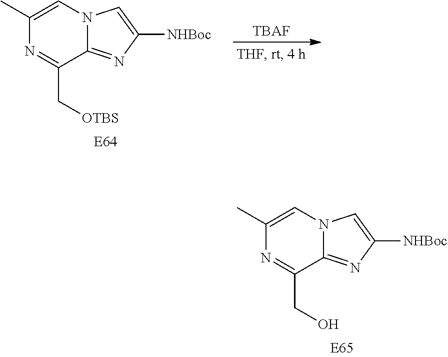 [SMILES: Cc1cn2cc(NC(=O)OC(C)(C)C)nc2c(CO[Si](C)(C)C(C)(C)C)n1 ; "TBAF" ; Cc1cn2cc(NC(=O)OC(C)(C)C)nc2c(CO)n1]

To a stirred mixture of tert-butyl N-[8-[[tert-butyl(dimethyl)silyl]oxymethyl]-6-methyl-imidazo[1,2-a]pyrazin-2-yl]carbamate (780 mg, 1.99 mmol) in THF (16 mL) was added TBAF (1.11 g, 3.97 mmol) at room temperature. The resulting mixture was stirred for 4 h at room temperature. The resulting mixture was diluted with water (20 mL). The resulting mixture was extracted with EA (3×20 mL). The combined organic layers were washed with water (3×60 mL) and brine (1×60 mL), dried over anhydrous Na₂SO₄. After filtration, the filtrate was concentrated under reduced pressure. The residue was purified by silica gel column chromatography, eluted with PE/EA (1:1) to afford tert-butyl N-[8-(hydroxymethyl)-6-methyl-imidazo[1,2-a]pyrazin-2-yl]carbamate (350 mg, 1.26 mmol, 63% yield) as a solid. LCMS (ES, m/z): 279 [M+H]⁺.

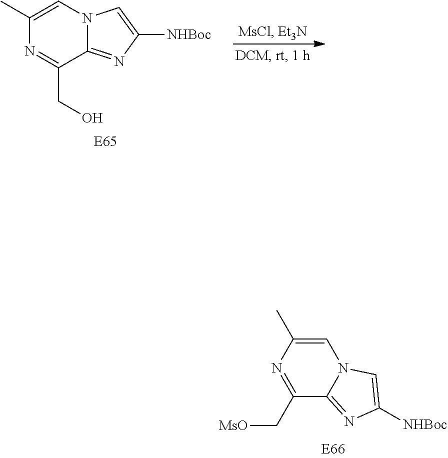

To a stirred mixture of tert-butyl N-[8-(hydroxymethyl)-6-methyl-imidazo[1,2-a]pyrazin-2-yl]carbamate (100 mg, 359.32 μmol) in DCM (2 mL) were added Et₃N (108.8 mg, 1.08 mmol) and MsCl (61.4 mg, 538.97 μmol) at 0° C. The resulting mixture was stirred for 1 h at room temperature. The reaction was quenched with water at 0° C. The resulting mixture was diluted with water (3 mL). The resulting mixture was extracted with DCM (3×3 mL). The combined organic layers were washed with water (3×10 mL) and brine (1×10 mL), dried over anhydrous Na₂SO₄. After filtration, the filtrate was concentrated under reduced pressure to afford [2-(tert-butoxycarbonylamino)-6-methyl-imidazo[1,2-a]pyrazin-8-yl]methyl methanesulfonate (150 mg, crude) as a solid. LCMS (ES, m/z): 357 [M+H]⁺.

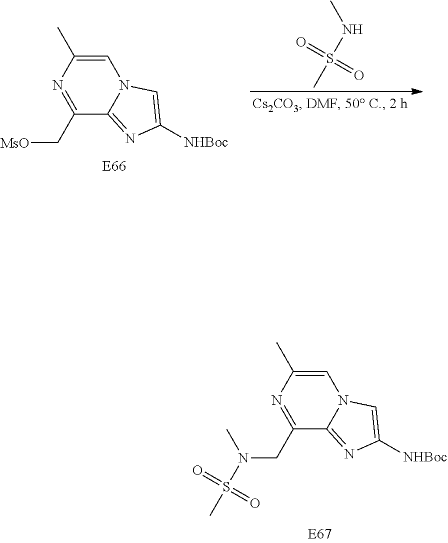

To a stirred mixture of [2-(tert-butoxycarbonylamino)-6-methyl-imidazo[1,2-a]pyrazin-8-yl]methyl methanesulfonate (150.0 mg, 420.88 μmol) and N-methylmethanesulfonamide (91.8 mg, 841.76 μmol) in DMF (3 mL) was added Cs₂CO₃ (411.4 mg, 1.26 mmol) at room temperature. The resulting mixture was stirred for 2 h at 50° C. The resulting mixture was diluted with water (5 mL). The resulting mixture was extracted with EA (3×5 mL). The combined organic layers were washed with water (3×15 mL) and brine (1×15 mL), dried over anhydrous Na₂SO₄. After filtration, the filtrate was concentrated under reduced pressure. The residue was purified by silica gel column chromatography, eluted with PE/EA (1:1) to afford tert-butyl N-[6-methyl-8-[[methyl(methylsulfonyl)amino]methyl]imidazo[1,2-a]pyrazin-2-yl]carbamate (100 mg, 270.68 μmol, 64% yield) as a solid. LCMS (ES, m/z): 370 [M+H]⁺.

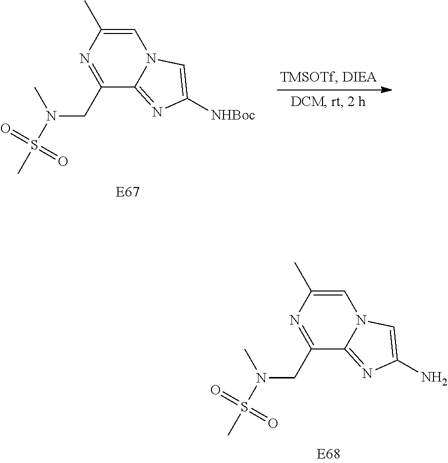 [SMILES: Cc1cn2cc(NC(=O)OC(C)(C)C)nc2c(CN(C)S(C)(=O)=O)n1 ; Cc1cn2cc(N)nc2c(CN(C)S(C)(=O)=O)n1]

To a stirred mixture of tert-butyl N-[6-methyl-8-[[methyl(methylsulfonyl)amino]methyl]imidazo[1,2-a]pyrazin-2-yl]carbamate (110 mg, 297.75 μmol) in DCM (2.5 mL) were added DIEA (153.9 mg, 1.19 mmol) and TMSOTf (198.5 mg, 893.25 μmol) at room temperature. The resulting mixture was stirred for 2 h at room temperature. The reaction was quenched with water at 0° C. The resulting mixture was diluted with water (3 mL). The resulting mixture was extracted with DCM (3×3 mL). After filtration, the filtrate was concentrated under reduced pressure. The residue was purified by silica gel column chromatography, eluted with DCM/MeOH (10:1) to afford N-[(2-amino-6-methyl-imidazo[1,2-a]pyrazin-8-yl)methyl]-N-methyl-methanesulfonamide (80 mg, 297.04 μmol, 99% yield) as a yellow solid. LCMS (ES, m/z): 270 [M+H]⁺.

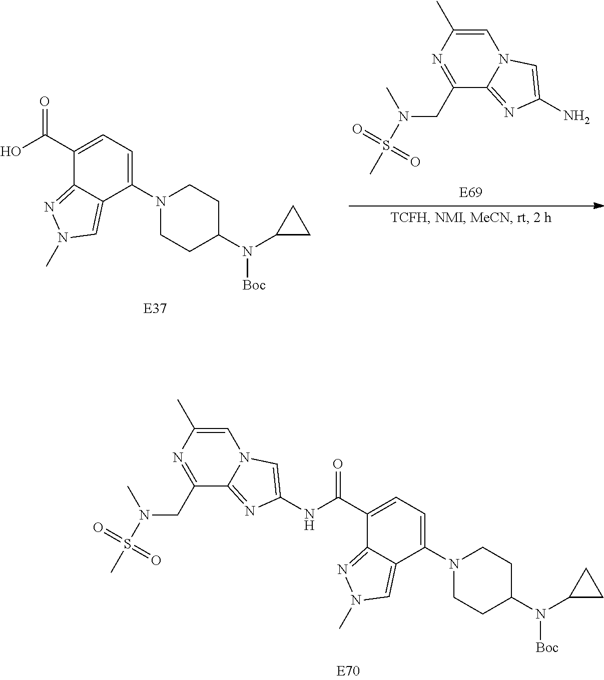 [SMILES: Cn1cc2c(N3CCC(N(C(=O)OC(C)(C)C)C4CC4)CC3)ccc(C(=O)O)c2n1 ; Cc1cn2cc(N)nc2c(CN(C)S(C)(=O)=O)n1 ; Cc1cn2cc(NC(=O)c3ccc(N4CCC(N(C(=O)OC(C)(C)C)C5CC5)CC4)c4cn(C)nc34)nc2c(CN(C)S(C)(=O)=O)n1]

To a stirred mixture of 4-[4-[tert-butoxycarbonyl(cyclopropyl)amino]-1-piperidyl]-2-methyl-indazole-7-carboxylic acid (100 mg, 241.26 μmol) and N-[(2-amino-6-methyl-imidazo[1,2-a]pyrazin-8-yl)methyl]-N-methyl-methanesulfonamide (77.9 mg, 289.51 μmol) in MeCN (2 mL) were added TCFH (101.5 mg, 361.88 μmol) and NMI (59.4 mg, 723.77 μmol) at room temperature. The resulting mixture was stirred for 2 h at room temperature. The resulting mixture was diluted with water (10 mL). The precipitated solids were collected by filtration and washed with water (3×10 mL) to afford tert-butyl N-cyclopropyl-N-[1-[2-methyl-7-[[6-methyl-8-[[methyl(methylsulfonyl)amino]methyl]imidazo[1,2-a]pyrazin-2-yl]carbamoyl]indazol-4-yl]-4-piperidyl]carbamate (80 mg, 120.16 μmol, 50% yield) as a solid. LCMS (ES, m/z): 666 [M+H].

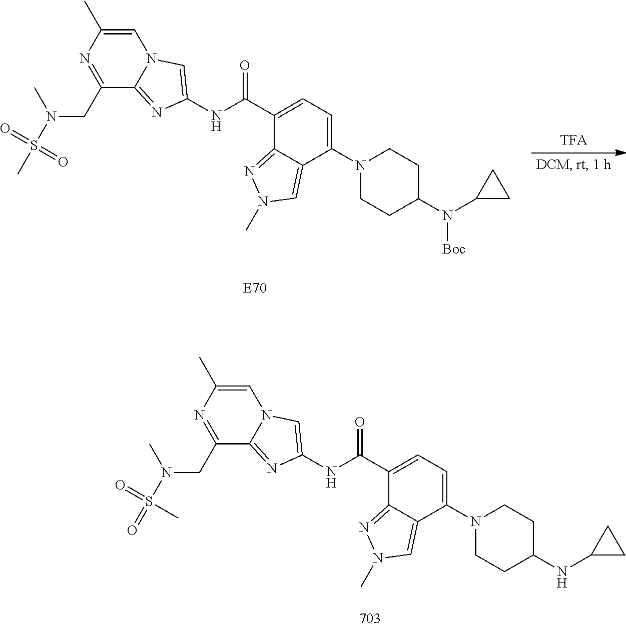 [SMILES: Cc1cn2cc(NC(=O)c3ccc(N4CCC(N(C(=O)OC(C)(C)C)C5CC5)CC4)c4cn(C)nc34)nc2c(CN(C)S(C)(=O)=O)n1 ; Cc1cn2cc(NC(=O)c3ccc(N4CCC(NC5CC5)CC4)c4cn(C)nc34)nc2c(CN(C)S(C)(=O)=O)n1]

To a stirred mixture of tert-butyl N-cyclopropyl-N-[1-[2-methyl-7-[[6-methyl-8-[[methyl(methylsulfonyl)amino]methyl]imidazo[1,2-a]pyrazin-2-yl]carbamoyl]indazol-4-yl]-4-piperidyl]carbamate (80 mg, 120.16 μmol) in DCM (2 mL) was added TFA (0.4 mL) dropwise at room temperature. The resulting mixture was stirred for 1 h at room temperature. The resulting mixture were concentrated under reduced pressure. The crude product was purified by Prep-HPLC with the following conditions (column, C18 silica gel, XBridge, 19×150 mm; mobile phase, MeCN in water (0.05% TFA), 40% to 80% gradient in 7 min; detector, UV 254 nm) to afford 4-(4-(cyclopropylamino)piperidin-1-yl)-2-methyl-N-(6-methyl-8-((N-methylmethylsulfonamido)methyl)imidazo[1,2-a]pyrazin-2-yl)-2H-indazole-7-carboxamide 2,2,2-trifluoroacetate (35.4 mg, 52.08 μmol, 43% yield) as a solid. LCMS (ES, m/z): 566 [M+H]⁻. ¹H NMR (300 MHz, DMSO-d₆) δ 11.56 (s, 1H), 8.89 (s, 1H), 8.66 (s, 2H), 8.50 (d, J=6.7 Hz, 2H), 8.07 (d, J=8.0 Hz, 1H), 6.59 (d, J=8.2 Hz, 1H), 4.87 (s, 2H), 4.31 (s, 3H), 4.07 (d, J=12.7 Hz, 2H), 3.55-3.47 (m, 1H), 3.14 (s, 3H), 3.05 (d, J=12.7 Hz, 2H), 2.94 (s, 3H), 2.85-2.76 (m, 1H), 2.55 (s, 1H), 2.46 (s, 3H), 2.20 (d, J=11.7 Hz, 2H), 1.73 (d, J=11.9 Hz, 2H), 0.85 (dd, J=10.5, 4.0 Hz, 4H).

### Example 235: Synthesis of Compound 704

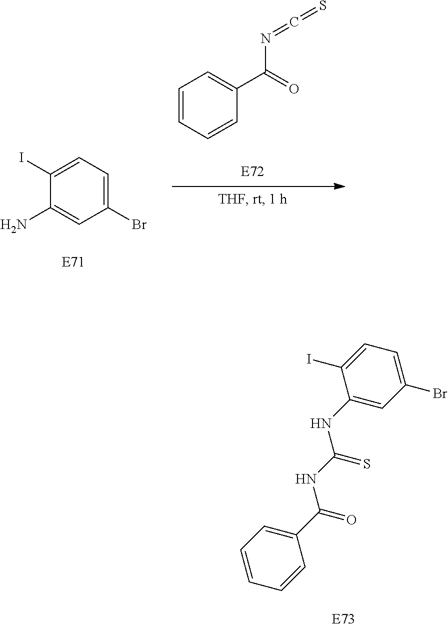 [SMILES: O=C(N=C=S)c1ccccc1 ; Nc1cc(Br)ccc1I ; O=C(NC(=S)Nc1cc(Br)ccc1I)c1ccccc1]

To a stirred mixture of 5-bromo-2-iodo-aniline (35.0 g, 117.48 mmol) in THF (500 mL) was added benzoyl isothiocyanate (19.2 g, 117.48 mmol) dropwise at room temperature. The resulting mixture was stirred for 1 h at rt. The resulting mixture was concentrated under reduced pressure. The residue was purified by trituration with MTBE (200 ml). The precipitated solids were collected by filtration and washed with MTBE (50 mL). This resulted in N-[(5-bromo-2-iodo-phenyl)carbamothioyl]benzamide (40 g, 86.75 mmol, 74% yield) as a solid. LCMS (ES, m/z): 461 [M+H]⁺.

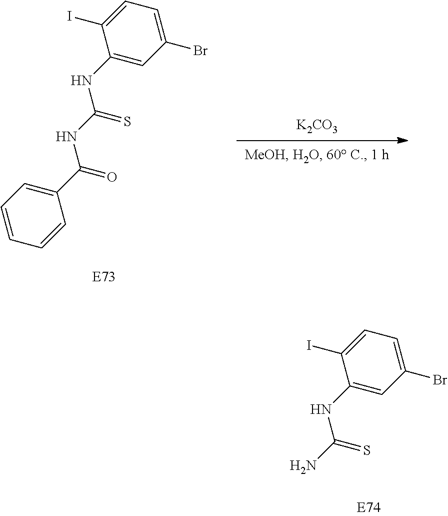 [SMILES: O=C(NC(=S)Nc1cc(Br)ccc1I)c1ccccc1 ; "K2CO3" ; NC(=S)Nc1cc(Br)ccc1I]

To a stirred mixture of N-[(5-bromo-2-iodo-phenyl)carbamothioyl]benzamide (40.0 g, 86.75 mmol) in MeOH (300 mL) and H₂O (300 mL) was added K₂CO₃ (23.9 g, 173.49 mmol) in portions at room temperature. The resulting mixture was stirred for 1 h at 60° C. under nitrogen atmosphere. The resulting mixture was concentrated under reduced pressure. The residue was purified by trituration with EA (200 mL). The precipitated solids were collected by filtration and washed with EA (50 mL). This resulted in (5-bromo-2-iodo-phenyl)thiourea (25 g, 70.03 mmol, 81% yield) as a solid. LCMS (ES, m/z): 357 [M+H]⁺.

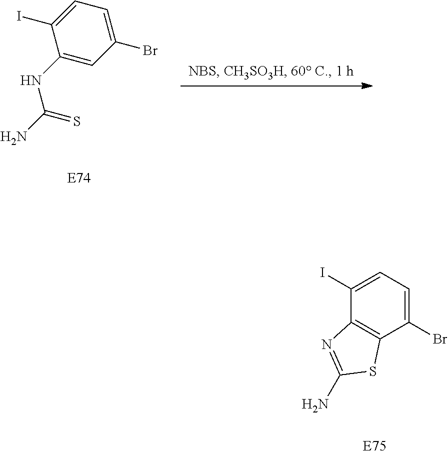 [SMILES: NC(=S)Nc1cc(Br)ccc1I ; "NBS, CH3SO3H, 60° C., 1 h" ; Nc1nc2c(I)ccc(Br)c2s1]

To a stirred mixture of (5-bromo-2-iodo-phenyl)thiourea (23.0 g, 64.42 mmol) in CH₃SO₃H (70 mL) was added NBS (12.6 g, 70.87 mmol) in portions at room temperature. The resulting mixture was stirred for 1 h at 60° C. under nitrogen atmosphere. The reaction was quenched with water (500 mL) at 0° C. The mixture was acidified to pH 7 with NaOH. The resulting mixture was extracted with EA (500 mL), and the organic layer was dried over anhydrous Na₂SO₄. After filtration, the filtrate was concentrated under reduced pressure. The residue was purified by trituration with EA (150 mL). The precipitated solids were collected by filtration and washed with EA (50 mL). This resulted in 7-bromo-4-iodo-1,3-benzothiazol-2-amine (16 g, 45.07 mmol, 70% yield) as a solid. LCMS (ES, m/z): 355 [M+H]⁺.

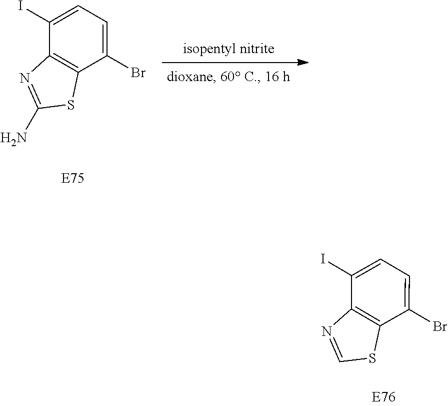 [SMILES: Nc1nc2c(I)ccc(Br)c2s1 ; "isopentyl nitrite" ; Brc1ccc(I)c2ncsc12]

To a stirred mixture of 7-bromo-4-iodo-1,3-benzothiazol-2-amine (16.0 g, 45.07 mmol) in Dioxane (250 mL) was added isopentyl nitrite (9.5 g, 81.13 mmol) dropwise at room temperature. The resulting mixture was stirred for 24 h at 60° C. The reaction was quenched with water (500 mL) at rt. The resulting mixture was extracted with EA (2×500 mL). The combined organic layers were washed with brine (1×500 mL), dried over anhydrous Na₂SO₄. After filtration, the filtrate was concentrated under reduced pressure. The residue was purified by silica gel column chromatography, eluted with DCM/EA (20:1) to afford 7-bromo-4-iodo-1,3-benzothiazole (12 g, 35.30 mmol, 78% yield) as a solid. LCMS (ES, m/z): 340 [M+H].

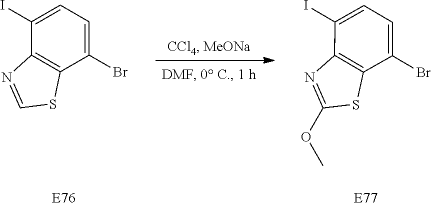

To a stirred mixture of 7-bromo-4-iodo-1,3-benzothiazole (12.0 g, 35.30 mmol) in DMF (120 mL) was added MeONa (5.7 g, 105.89 mmol), CCl₄ (5.9 g, 38.83 mmol) at room temperature. The resulting mixture was stirred for 1 h at 0° C. The reaction was quenched with water (300 mL) at 0° C. The resulting mixture was extracted with EA (2×500 mL). The combined organic layers were washed with brine (1×500 mL), dried over anhydrous Na₂SO₄. After filtration, the filtrate was concentrated under reduced pressure. The residue was purified by silica gel column chromatography, eluted with PE/DCM (5:1) to afford 7-bromo-4-iodo-2-methoxy-1,3-benzothiazole (3.2 g, 8.65 mmol, 25% yield) as a solid. LCMS (ES, m/z): 370 [M+H]⁺.

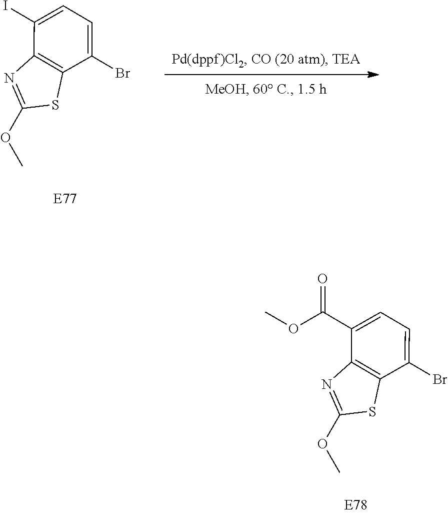 [SMILES: COc1nc2c(I)ccc(Br)c2s1 ; "Pd(dppf)Cl2, CO (20 atm), TEA" ; COC(=O)c1ccc(Br)c2sc(OC)nc12]

To a solution of 7-bromo-4-iodo-2-methoxy-1,3-benzothiazole (3.1 g, 8.38 mmol) in 50 mL of MeOH was added Pd(dppf)Cl₂ (613.0 mg, 837.83 μmol) and TEA (4.2 g, 41.89 mmol) in a pressure tank. The mixture was purged with nitrogen for 2 min and then was pressurized to 2 Mpa with carbon monoxide at 60° C. for 2 h. The reaction mixture was cooled to room temperature and filtered to remove insoluble solids, the filtrate was concentrated under reduced pressure. The residue was purified by silica gel column chromatography, eluted with PE/EA (5:1) to afford methyl 7-bromo-2-methoxy-1,3-benzothiazole-4-carboxylate (2.4 g, 7.94 mmol, 95% yield) as a solid. LCMS (ES, m/z): 302 [M+H]⁺.

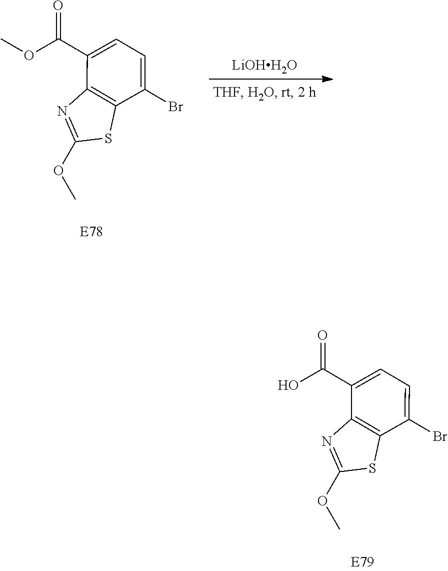 [SMILES: COC(=O)c1ccc(Br)c2sc(OC)nc12 ; "LiOH·H2O" ; COc1nc2c(C(=O)O)ccc(Br)c2s1]

To a stirred solution of methyl 7-bromo-2-methoxy-1,3-benzothiazole-4-carboxylate (340.0 mg, 1.13 mmol) in THF (3 mL), H₂O (3 mL) was added LiOH·H₂O (236.1 mg, 5.63 mmol) in portions at rt. The resulting mixture was stirred for 2 h at rt. The resulting mixture was diluted with water (20 mL). The mixture was acidified to pH 6 with HCl (2N). The resulting mixture was extracted with EA (2×20 mL). The combined organic layers were washed with brine (1×20 mL), dried over anhydrous Na₂SO₄. After filtration, the filtrate was concentrated under reduced pressure. This resulted in 7-bromo-2-methoxy-1,3-benzothiazole-4-carboxylic acid (310 mg, 1.08 mmol, 96% yield) as a solid. LCMS (ES, m/z): 288 [M+H]⁻

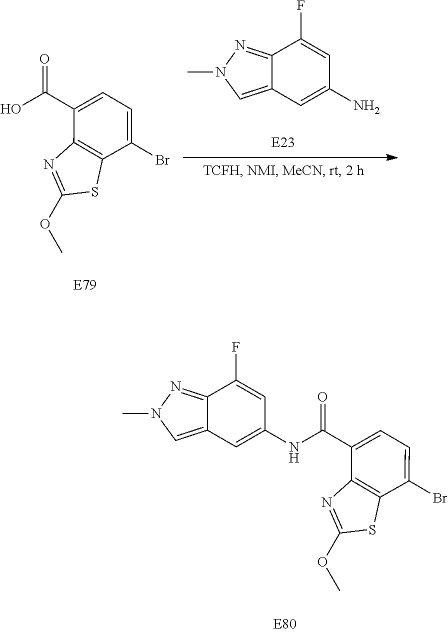 [SMILES: COc1nc2c(C(=O)O)ccc(Br)c2s1 ; Cn1cc2cc(N)cc(F)c2n1 ; COc1nc2c(C(=O)Nc3cc(F)c4nn(C)cc4c3)ccc(Br)c2s1]

To a stirred mixture of 7-bromo-2-methoxy-1,3-benzothiazole-4-carboxylic acid (310.0 mg, 1.08 mmol) and 7-fluoro-2-methyl-indazol-5-amine (213.2 mg, 1.29 mmol) in MeCN (6 mL) was added NMI (441.6 mg, 5.38 mmol) and TCFH (422.6 mg, 1.51 mmol) at room temperature. The resulting mixture was stirred for 2 h at rt. The resulting mixture was diluted with water (10 mL). The precipitated solids were collected by filtration and washed with MeCN (2 ml). This resulted in 7-bromo-N-(7-fluoro-2-methyl-indazol-5-yl)-2-methoxy-1,3-benzothiazole-4-carboxamide (420.0 mg, 964.92 μmol, 90% yield) as a solid. LCMS (ES, m/z): 435 [M+H]⁺.

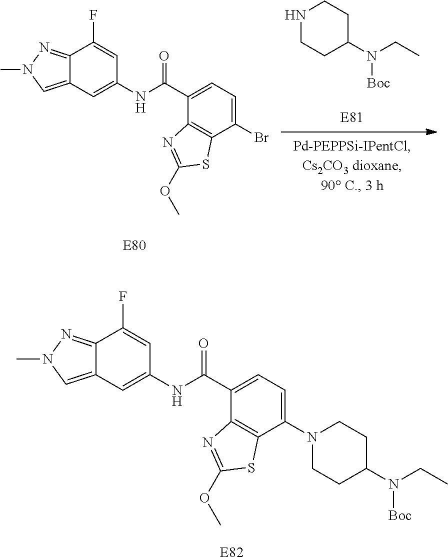

To a stirred mixture of 7-bromo-N-(7-fluoro-2-methyl-indazol-5-yl)-2-methoxy-1,3-benzothiazole-4-carboxamide (100.0 mg, 229.74 μmol) and tert-butyl N-ethyl-N-(4-piperidyl)carbamate (52.5 mg, 229.74 μmol) in Dioxane (3 mL) were added Cs₂CO₃ (149.7 mg, 459.49 μmol), Pd-PEPPSI-IPentCl (19.3 mg, 22.97 μmol) at room temperature under nitrogen atmosphere. The resulting mixture was stirred for 3 h at 90° C. under nitrogen atmosphere. The mixture was allowed to cool down to room temperature. The resulting mixture was diluted with water (10 mL). The resulting mixture was extracted with EA (2×20 mL). The combined organic layers were washed with water (2×40 mL), brine (1×40 mL), dried over anhydrous Na₂SO₄. After filtration, the filtrate was concentrated under reduced pressure. The residue was purified by silica gel column chromatography, eluted with DCM/EA (1:3) to afford tert-butyl N-ethyl-N-[1-[4-[(7-fluoro-2-methyl-indazol-5-yl)carbamoyl]-2-methoxy-1,3-benzothiazol-7-yl]-4-piperidyl]carbamate (80 mg, 137.29 μmol, 60% yield) as a solid. LCMS (ES, m/z): 583 [M+H]⁺.

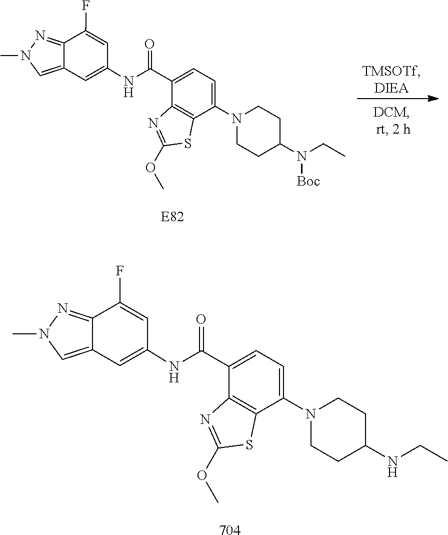

To a solution of tert-butyl N-ethyl-N-[1-[4-[(7-fluoro-2-methyl-indazol-5-yl)carbamoyl]-2-methoxy-1,3-benzothiazol-7-yl]-4-piperidyl]carbamate (70.0 mg, 120.13 μmol) in DCM (1 mL) was added TMSOTf (80.1 mg, 360.40 μmol, 65.23 μL), DIEA (31.0 mg, 240.27 μmol). The reaction was stirred for 1 h at rt. The resulting mixture was concentrated under reduced pressure and adjusted to pH 8 with saturated NaHCO₃ (aq.). The resulting mixture was diluted with water (5 mL) and extracted with DCM/MeOH (20/1) (2×10 mL). The combined organic layers were washed with water (1×20 mL), brine (1×20 mL) and dried over anhydrous Na₂SO₄. After filtration, the filtrate was concentrated under reduced pressure. The residue was purified by reversed-phase flash chromatography with the following conditions (column, C18 silica gel; mobile phase, CH₃CN in water (0.1% TFA), 25% to 50% gradient in 10 min; detector, UV 254 nm) to afford 7-(4-(ethylamino)piperidin-1-yl)-N-(7-fluoro-2-methyl-2H-indazol-5-yl)-2-methoxybenzo[d]thiazole-4-carboxamide 2,2,2-trifluoroacetate (24 mg, 40.23 μmol, 33% yield) as an solid. LCMS (ES, m/z): 483 [M+H]⁺. ¹H NMR (400 MHz, DMSO-d₆) δ 11.39 (s, 1H), 8.51 (s, 2H), 8.44 (d, J=2.8 Hz, 1H), 8.15 (d, J=8.5 Hz, 1H), 8.11 (s, 1H), 7.36 (d, J=13.0 Hz, 1H), 7.13 (d, J=8.6 Hz, 1H), 4.42 (s, 3H), 4.20 (s, 3H), 3.67 (d, J=12.4 Hz, 2H), 3.36 (s, 1H), 3.08-3.02 (m, 2H), 2.93 (t, J=12.1 Hz, 2H), 2.17 (d, J=12.2 Hz, 2H), 1.69 (q, J=11.7 Hz, 2H), 1.23 (t, J=7.2 Hz, 3H).

### Example 236: Synthesis of Compound 705

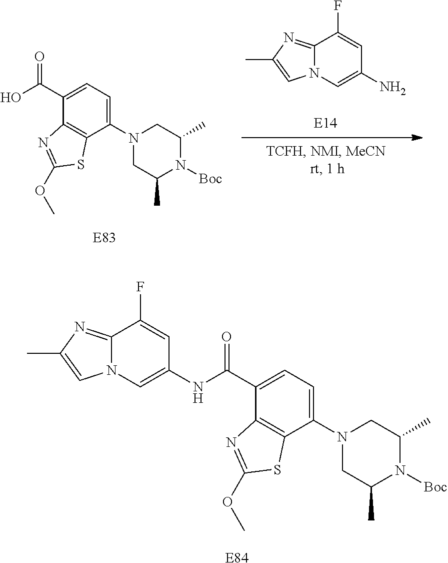

To a stirred mixture of 7-[(3S,5S)-4-tert-butoxycarbonyl-3,5-dimethyl-piperazin-1-yl]-2-methoxy-1,3-benzothiazole-4-carboxylic acid (60 mg, 142.35 μmol) and 8-fluoro-2-methyl-imidazo[1,2-a]pyridin-6-amine (28.2 mg, 170.81 μmol) in MeCN (1.5 mL) were added NMI (35.0 mg, 427.04 μmol) and TCFH (59.9 mg, 213.52 μmol) at room temperature. The resulting mixture was stirred for 2 h at rt. The resulting mixture was concentrated under reduced pressure. The residue was purified by silica gel column chromatography, eluted with PE/EA (1:3) to afford tert-butyl (2S,6S)-4-[4-[(8-fluoro-2-methyl-imidazo[1,2-a]pyridin-6-yl)carbamoyl]-2-methoxy-1,3-benzothiazol-7-yl]-2,6-dimethyl-piperazine-1-carboxylate (70 mg, 123.10 μmol, 86% yield) as a solid. LCMS (ES, m/z): 569 [M+H]⁺.

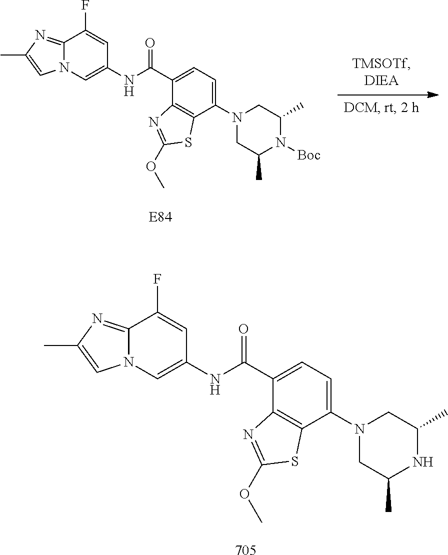

To a stirred mixture of tert-butyl (2S,6S)-4-[4-[(8-fluoro-2-methyl-imidazo[1,2-a]pyridin-6-yl)carbamoyl]-2-methoxy-1,3-benzothiazol-7-yl]-2,6-dimethyl-piperazine-1-carboxylate (70 mg, 123.10 μmol) in DCM (1.5 mL) was added DIEA (32 mg, 246.19 μmol) and TMSOTf (82 mg, 369.29 μmol) dropwise at room temperature. The resulting mixture was stirred for 2 h at room temperature. The resulting mixture were concentrated under reduced pressure and diluted with water (2 mL). The mixture was basified to pH 8 with saturated NaHCO₃ (aq.) and extracted with DCM (3×3 mL). The combined organic layers were washed with water (2×10 mL) and brine (1×10 mL), dried over anhydrous Na₂SO₄. After filtration, the filtrate was concentrated under reduced pressure. The crude product was purified by Prep-HPLC with the following conditions (column, C18 silica gel, XBridge, 19×150 mm; mobile phase, MeCN in water (0.05% NH₃·H₂O), 20% to 50% gradient in 7 min; detector, UV 254 nm) to afford 7-[(3S,5S)-3,5-dimethylpiperazin-1-yl]-N-(8-fluoro-2-methyl-imidazo[1,2-a]pyridin-6-yl)-2-methoxy-1,3-benzothiazole-4-carboxamide (11.5 mg, 24.54 μmol, 20% yield) as a solid. LCMS (ES, m/z): 469 [M+H]⁺. ¹H NMR (400 MHz, DMSO-d₆) δ 11.27 (s, 1H), 9.22 (d, J=1.5 Hz, 1H), 8.10 (d, J=8.5 Hz, 1H), 7.92 (d, J=3.1 Hz, 1H), 7.27-7.19 (m, 1H), 7.06 (d, J=8.6 Hz, 1H), 4.41 (s, 3H), 3.12 (d, J=11.9 Hz, 2H), 2.93 (dd, J=11.4, 6.1 Hz, 2H), 2.35 (s, 3H), 1.17 (d, J=6.4 Hz, 6H).

### Example 237: Synthesis of Compound 706

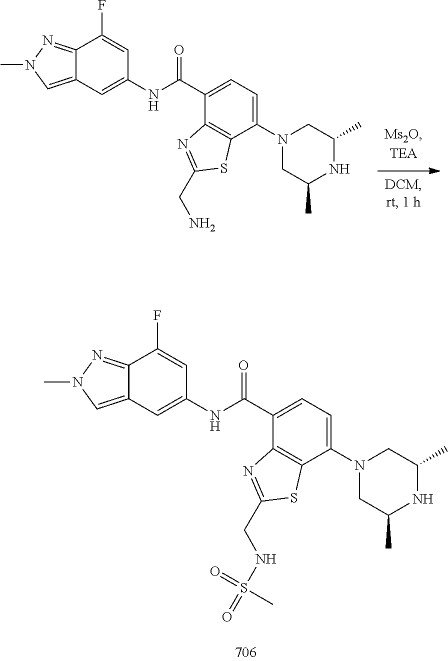

To a solution of 2-(aminomethyl)-7-[(3S,5S)-3,5-dimethylpiperazin-1-yl]-N-(7-fluoro-2-methyl-indazol-5-yl)-1,3-benzothiazole-4-carboxamide (0.11 g, 235.26 μmol) in DCM (4 mL) was added TEA (71 mg, 705.79 μmol) and Ms₂O (41 mg, 235.26 μmol). The reaction was stirred for 1 h at rt. The resulting was concentrated under reduced pressure. The residue was purified by reversed-phase flash chromatography with the following conditions (column, C18 silica gel; mobile phase, CH₃CN in water (0.1% NH₃·H₂O), 35% to 70% gradient in 10 min; detector, UV 254 nm) to afford 7-[(3S,5S)-3,5-dimethylpiperazin-1-yl]-N-(7-fluoro-2-methyl-indazol-5-yl)-2-(methanesulfonamidomethyl)-1,3-benzothiazole-4-carboxamide (0.03 g, 54.98 μmol, 23% yield) as a solid. LCMS (ES, m/z): 546 [M+H]⁺. ¹H NMR (400 MHz, DMSO-d₆) δ 11.84 (s, 1H), 8.45 (d, J=2.8 Hz, 1H), 8.36 (s, 1H), 8.26-8.17 (m, 2H), 7.47 (dd, J=13.1, 1.6 Hz, 1H), 7.15 (d, J=8.4 Hz, 1H), 4.88 (s, 2H), 4.20 (s, 3H), 3.26 (q, J=5.7, 4.6 Hz, 2H), 3.19 (dd, J=11.4, 3.2 Hz, 2H), 3.09 (s, 3H), 2.99 (dd, J=11.4, 6.0 Hz, 2H), 1.19 (d, J=6.4 Hz, 6H).

### Example 238: Synthesis of Compound 707

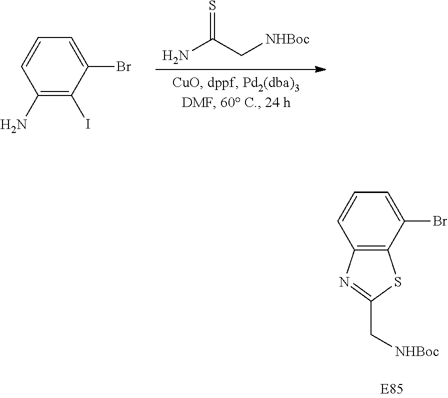 [SMILES: Nc1cccc(Br)c1I ; CC(C)(C)OC(=O)NCC(N)=S ; CC(C)(C)OC(=O)NCc1nc2cccc(Br)c2s1]

To a stirred mixture of 3-bromo-2-iodo-aniline (4 g, 13.43 mmol) and tert-butyl N-(2-amino-2-thioxo-ethyl)carbamate (2.55 g, 13.43 mmol) in DMF (40 mL) were added CuO (1.07 g, 13.43 mmol), dppf (3.72 g, 6.71 mmol) and Pd₂(dba)₃ (2.46 g, 2.69 mmol) at room temperature under nitrogen atmosphere. The resulting mixture was stirred for 24 h at 60° C. under nitrogen atmosphere. The mixture was allowed to cool down to room temperature. The resulting mixture was diluted with water (50 mL). The resulting mixture was extracted with EA (3×50 mL). The combined organic layers were washed with water (3×80 mL), brine (1×100 mL), dried over anhydrous Na₂SO₄. After filtration, the filtrate was concentrated under reduced pressure. The residue was purified by silica gel column chromatography, eluted with PE/EA (10:1) to afford tert-butyl N-[(7-bromo-1,3-benzothiazol-2-yl)methyl]carbamate (2.5 g, 7.28 mmol, 54% yield) as a solid. LCMS (ES, m/z): 343 [M+H]⁺.

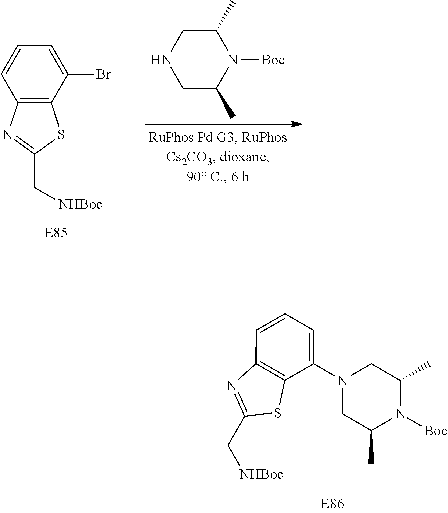 [SMILES: CC(C)(C)OC(=O)NCc1nc2cccc(Br)c2s1 ; C[C@H]1CNC[C@H](C)N1C(=O)OC(C)(C)C ; C[C@H]1CN(c2cccc3nc(CNC(=O)OC(C)(C)C)sc23)C[C@H](C)N1C(=O)OC(C)(C)C]

To a stirred mixture of tert-butyl N-[(7-bromo-1,3-benzothiazol-2-yl)methyl]carbamate (2 g, 5.83 mmol) and tert-butyl (2S,6S)-2,6-dimethylpiperazine-1-carboxylate (1.50 g, 6.99 mmol) in Dioxane (20 mL) were added Cs₂CO₃ (3.80 g, 11.65 mmol), Ruphos (543.8 mg, 1.17 mmol) and RuPhos Pd G3 (487.4 mg, 582.68 μmol) at room temperature under nitrogen atmosphere. The resulting mixture was stirred for 6 h at 90° C. under nitrogen atmosphere. The mixture was allowed to cool down to room temperature. After filtration, the filtrate was concentrated under reduced pressure. The residue was purified by silica gel column chromatography, eluted with PE/EA (5:1) to afford tert-butyl (2S,6S)-4-[2-[(tert-butoxycarbonylamino)methyl]-1,3-benzothiazol-7-yl]-2,6-dimethyl-piperazine-1-carboxylate (1.83 g, 3.84 mmol, 66% yield) as a solid LCMS (ES, m/z): 477 [M+H]⁺.

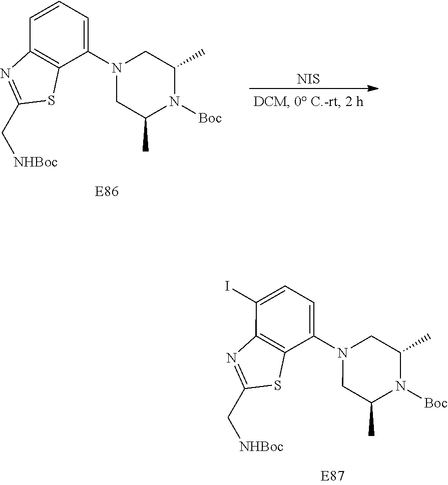 [SMILES: C[C@H]1CN(c2cccc3nc(CNC(=O)OC(C)(C)C)sc23)C[C@H](C)N1C(=O)OC(C)(C)C ; "NIS" ; C[C@H]1CN(c2ccc(I)c3nc(CNC(=O)OC(C)(C)C)sc23)C[C@H](C)N1C(=O)OC(C)(C)C]

To a solution of tert-butyl (2S,6S)-4-[2-[(tert-butoxycarbonylamino)methyl]-1,3-benzothiazol-7-yl]-2,6-dimethyl-piperazine-1-carboxylate (1.8 g, 3.78 mmol) in DCM (20 mL) was added NIS (849.6 mg, 3.78 mmol). The reaction was stirred for 2 h at rt. The resulting mixture was diluted with water (20 mL) and extracted with EA (3×10 mL). The combined organic layers were washed with water (1×20 mL), brine (1×20 mL) and dried over anhydrous Na₂SO₄. After filtration, the filtrate was concentrated under reduced pressure. The residue was purified by silica gel column chromatography, eluted with PE/EA (1:1) to afford tert-butyl (2S,6S)-4-[2-[(tert-butoxycarbonylamino)methyl]-4-iodo-1,3-benzothiazol-7-yl]-2,6-dimethyl-piperazine-1-carboxylate (1.5 g, 2.49 mmol, 66% yield) as a solid. LCMS (ES, m/z): 603 [M+H]⁺.

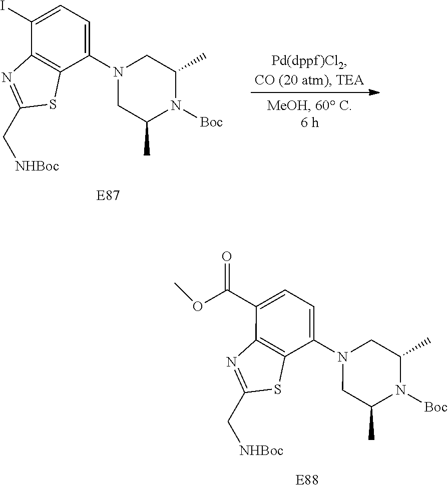 [SMILES: C[C@H]1CN(c2ccc(I)c3nc(CNC(=O)OC(C)(C)C)sc23)C[C@H](C)N1C(=O)OC(C)(C)C ; "Pd(dppf)Cl2, CO (20 atm), TEA" ; COC(=O)c1ccc(N2C[C@H](C)N(C(=O)OC(C)(C)C)[C@@H](C)C2)c2sc(CNC(=O)OC(C)(C)C)nc12]

To a solution of tert-butyl (2S,6S)-4-[2-[(tert-butoxycarbonylamino)methyl]-4-iodo-1,3-benzothiazol-7-yl]-2,6-dimethyl-piperazine-1-carboxylate (1.5 g, 2.49 mmol) in MeOH (15 mL) was added TEA (755.3 mg, 7.47 mmol) and Pd(dppf)Cl₂ (182.6 mg, 248.95 μmol) in a pressure tank. The mixture was purged with nitrogen for 10 minutes and then was pressurized to 20 atm with carbon monoxide at 60° C. for 6 h. The reaction mixture was cooled to room temperature and filtered to remove insoluble solids. The filtrate was concentrated under reduced pressure. The residue was purified by silica gel column chromatography, eluted with PE:EA (1:8) to afford methyl 2-[(tert-butoxycarbonylamino)methyl]-7-[(3S,5S)-4-tert-butoxycarbonyl-3,5-dimethyl-piperazin-1-yl]-1,3-benzothiazole-4-carboxylate (0.9 g, 1.68 mmol, 68% yield) as a solid. LCMS (ES, m/z): 535 [M+H]⁺.

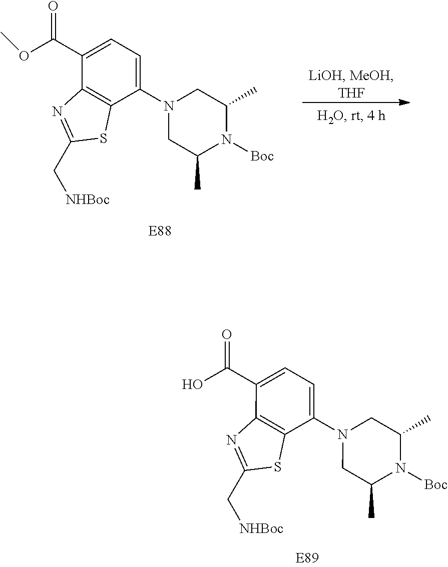

To a stirred mixture of methyl 2-[(tert-butoxycarbonylamino)methyl]-7-[(3S,5S)-4-tert-butoxycarbonyl-3,5-dimethyl-piperazin-1-yl]-1,3-benzothiazole-4-carboxylate (0.9 g, 1.68 mmol) in MeOH (2 mL) THF (2 mL) and H₂O (1 mL) was added LiOH (201.6 mg, 8.42 mmol) at room temperature. The resulting mixture was stirred for 4 h at room temperature. The mixture residue was neutralized to pH 5 with 1 N of HCl (aq.). The resulting mixture was diluted with water (5 mL). The resulting mixture was extracted with EA (3×10 mL). The combined organic layers were washed with water (1×20 mL), brine (1×40 mL), dried over anhydrous Na₂SO₄. After filtration, the filtrate was concentrated under reduced pressure to afford 2-[(tert-butoxycarbonylamino)methyl]-7-[(3S,5S)-4-tert-butoxycarbonyl-3,5-dimethyl-piperazin-1-yl]-1,3-benzothiazole-4-carboxylic acid (0.86 g, 1.65 mmol, 98% yield) as a solid. LCMS (ES, m/z): 521 [M+H]⁺.

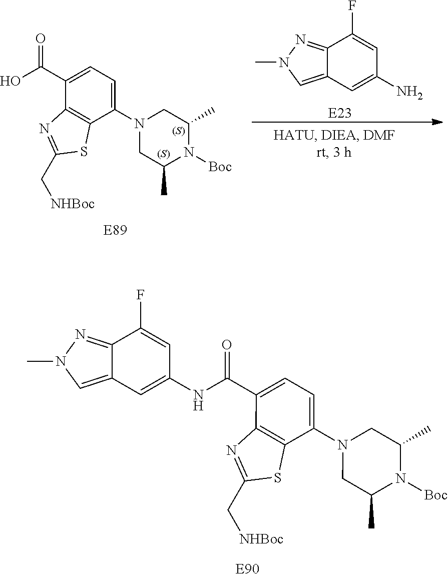

To a stirred mixture of 2-[(tert-butoxycarbonylamino)methyl]-7-[(3S,5S)-4-tert-butoxycarbonyl-3,5-dimethyl-piperazin-1-yl]-1,3-benzothiazole-4-carboxylic acid (0.84 g, 1.61 mmol) and 7-fluoro-2-methyl-indazol-5-amine (319.8 mg, 1.94 mmol) in DMF (10 mL) were added DIEA (625.6 mg, 4.84 mmol), HATU (920.9 mg, 2.42 mmol) at room temperature. The resulting mixture was stirred for 3 h at room temperature. The resulting mixture was diluted with water (10 mL). The resulting mixture was extracted with EA (3×20 mL). The combined organic layers were washed with water (1×40 mL), brine (1×40 mL), dried over anhydrous Na₂SO₄. After filtration, the filtrate was concentrated under reduced pressure to afford tert-butyl (2S,6S)-4-(2-(((tert-butoxycarbonyl)amino)methyl)-4-((7-fluoro-2-methyl-2H-indazol-5-yl)carbamoyl)benzo[d]thiazol-7-yl)-2,6-dimethylpiperazine-1-carboxylate (0.9 g, 1.35 mmol, 84% yield) as a solid. LCMS (ES, m/z): 668 [M+H]⁺.

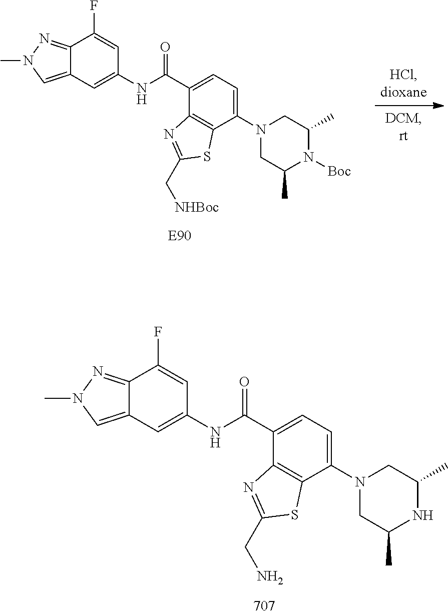

To a solution of tert-butyl (2S,6S)-4-[2-[(tert-butoxycarbonylamino)methyl]-4-[(7-fluoro-2-methyl-indazol-5-yl)carbamoyl]-1,3-benzothiazol-7-yl]-2,6-dimethyl-piperazine-1-carboxylate (0.07 g, 104.82 μmol) in DCM (2 mL) was added HCl in dioxane (1 mL). The reaction was stirred for 1 h at rt. The resulting mixture was concentrated under reduced pressure and adjusted to pH 8 with saturated NaHCO₃ (aq.). After filtration, the filtrate was concentrated under reduced pressure. The solution was purified by reversed-phase flash chromatography with the following conditions (column, C18 silica gel; mobile phase, CH₃CN in water (0.1% NH₃·H₂O), 35% to 75% gradient in 10 min; detector, UV 254 nm) to afford 2-(aminomethyl)-7-[(3S,5S)-3,5-dimethylpiperazin-1-yl]-N-(7-fluoro-2-methyl-indazol-5-yl)-1,3-benzothiazole-4-carboxamide (0.03 g, 64.16 μmol, 61% yield) as a solid. LCMS (ES, m/z): 468 [M+H]⁺. ¹H NMR (400 MHz, DMSO-d₆) δ 12.01 (s, 1H), 8.44 (d, J=2.8 Hz, 1H), 8.19 (d, J=8.4 Hz, 1H), 8.11 (d, J=1.6 Hz, 1H), 7.43 (dd, J=13.0, 1.6 Hz, 1H), 7.09 (d, J=8.4 Hz, 1H), 4.34 (s, 2H), 4.20 (s, 3H), 3.30-3.22 (m, 2H), 3.19 (dd, J=11.3, 3.1 Hz, 2H), 2.99 (dd, J=11.5, 6.1 Hz, 2H), 2.7 (S, 1.4H) 1.19 (d, J=6.5 Hz, 6H).

### Example 239: Synthesis of Compound 708

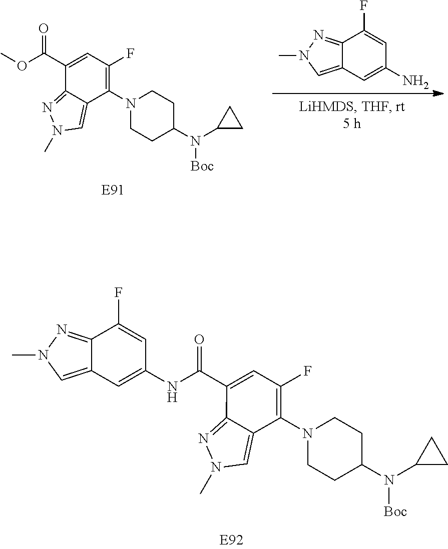 [SMILES: COC(=O)c1cc(F)c(N2CCC(N(C(=O)OC(C)(C)C)C3CC3)CC2)c2cn(C)nc12 ; Cn1cc2cc(N)cc(F)c2n1 ; Cn1cc2cc(NC(=O)c3cc(F)c(N4CCC(N(C(=O)OC(C)(C)C)C5CC5)CC4)c4cn(C)nc34)cc(F)c2n1]

To a stirred mixture of methyl 4-[4-[tert-butoxycarbonyl(cyclopropyl)amino]-1-piperidyl]-5-fluoro-2-methyl-indazole-7-carboxylate (0.15 g, 335.94 μmol) and 7-fluoro-2-methyl-indazol-5-amine (67 mg, 403.12 μmol) in THF (3 mL) were added LiHMDS (1.0 M in THF) (1.01 mL, 1.01 mmol) at room temperature under nitrogen atmosphere. The resulting mixture was stirred for 5 h at room temperature under nitrogen atmosphere. The resulting mixture was diluted with water (5 mL). The resulting mixture was extracted with EA (3×5 mL). The combined organic layers were washed with water (1×20 mL), brine (1×20 mL), dried over anhydrous Na₂SO₄. After filtration, the filtrate was concentrated under reduced pressure. The residue was purified by silica gel column chromatography, eluted with PE/EA (1:8) to afford tert-butyl N-cyclopropyl-N-[1-[5-fluoro-7-[(7-fluoro-2-methyl-indazol-5-yl)carbamoyl]-2-methyl-indazol-4-yl]-4-piperidyl]carbamate (0.14 g, 241.53 μmol, 72% yield) as a solid. LCMS (ES, m/z): 580 [M+H]⁺.

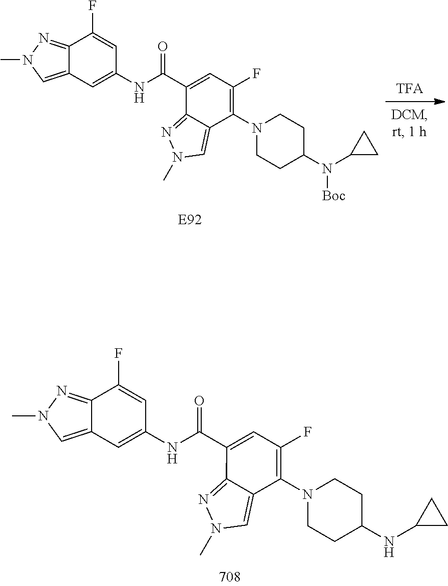 [SMILES: Cn1cc2cc(NC(=O)c3cc(F)c(N4CCC(N(C(=O)OC(C)(C)C)C5CC5)CC4)c4cn(C)nc34)cc(F)c2n1 ; Cn1cc2cc(NC(=O)c3cc(F)c(N4CCC(NC5CC5)CC4)c4cn(C)nc34)cc(F)c2n1]

To a solution of tert-butyl N-cyclopropyl-N-[1-[5-fluoro-7-[(7-fluoro-2-methyl-indazol-5-yl)carbamoyl]-2-methyl-indazol-4-yl]-4-piperidyl]carbamate (0.14 g, 241.53 μmol) in DCM (1 mL) was added TFA (0.4 mL). The reaction was stirred for 1 h at rt. The resulting mixture was concentrated under reduced pressure and adjusted to pH 8 with saturated NaHCO₃ (aq.). After filtration, the filtrate was concentrated under reduced pressure. The residue was purified by reversed-phase flash chromatography with the following conditions (column, C18 silica gel; mobile phase, CH₃CN in water (0.1% NH₃·H₂O), 45% to 85% gradient in 10 min; detector, UV 254 nm) to afford 4-[4-(cyclopropylamino)-1-piperidyl]-5-fluoro-N-(7-fluoro-2-methyl-indazol-5-yl)-2-methyl-indazole-7-carboxamide (0.02 g, 41.71 μmol, 17% yield) as a solid. LCMS (ES, m/z): 480 [M+H]⁺. ¹H NMR (400 MHz, DMSO-d₆) δ 11.11 (s, 1H), 8.79 (s, 1H), 8.43 (d, J=2.8 Hz, 1H), 8.07 (d, J=1.7 Hz, 1H), 7.80 (d, J=14.5 Hz, 1H), 7.42 (dd, J=13.1, 1.6 Hz, 1H), 4.31 (s, 3H), 4.20 (s, 3H), 3.74 (d, J=12.5 Hz, 2H), 3.23 (t, J=11.6 Hz, 2H), 2.80-2.70 (m, 1H), 2.24 (s, 1H), 2.18-2.06 (m, 1H), 2.04-1.96 (m, 2H), 1.50 (s, 1H), 1.47 (s, 1H), 0.40 (dt, J=6.2, 3.0 Hz, 2H), 0.29-0.21 (m, 2H).

### Example 240: Synthesis of Compound 709

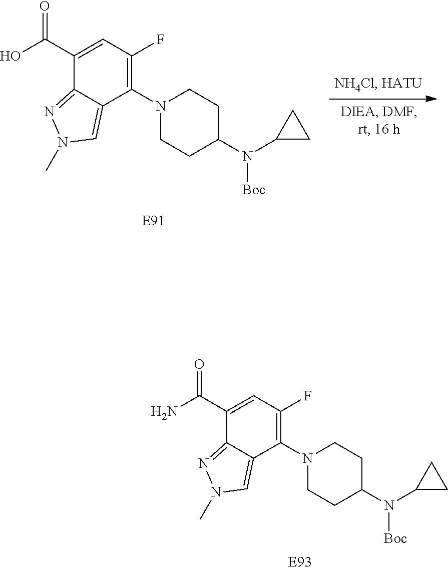 [SMILES: Cn1cc2c(N3CCC(N(C(=O)OC(C)(C)C)C4CC4)CC3)c(F)cc(C(=O)O)c2n1 ; Cn1cc2c(N3CCC(N(C(=O)OC(C)(C)C)C4CC4)CC3)c(F)cc(C(N)=O)c2n1]

To a stirred mixture of 4-[4-[tert-butoxycarbonyl(cyclopropyl)amino]-1-piperidyl]-5-fluoro-2-methyl-indazole-7-carboxylic acid (0.26 g, 601.17 μmol) and NH₄Cl (161 mg, 3.01 mmol) in DMF (5 mL) were added DIEA (233 mg, 1.80 mmol), HATU (343 mg, 901.76 μmol) at room temperature. The resulting mixture was stirred for 16 h at room temperature. The resulting mixture was diluted with water (5 mL). The resulting mixture was extracted with EA (3×5 mL). The combined organic layers were washed with water (1×20 mL), brine (1×20 mL), dried over anhydrous Na₂SO₄. After filtration, the filtrate was concentrated under reduced pressure. The residue was purified by silica gel column chromatography, eluted with PE/EA (1:8) to afford tert-butyl N-[1-(7-carbamoyl-5-fluoro-2-methyl-indazol-4-yl)-4-piperidyl]-N-cyclopropyl-carbamate (0.24 g, 556.20 μmol, 93% yield) as a solid. LCMS (ES, m/z): 432 [M+H]⁺.

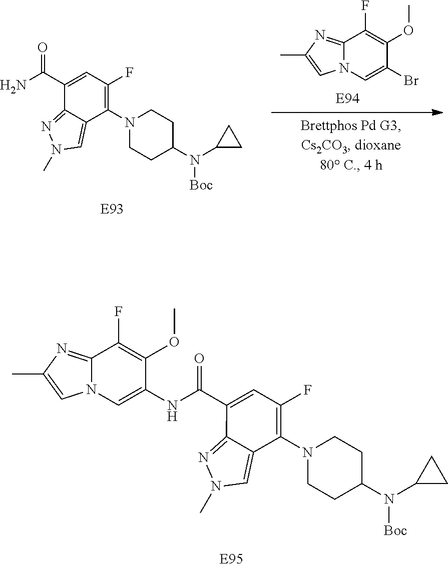

To a stirred mixture of tert-butyl N-[1-(7-carbamoyl-5-fluoro-2-methyl-indazol-4-yl)-4-piperidyl]-N-cyclopropyl-carbamate (0.12 g, 278.10 μmol) and 6-bromo-8-fluoro-7-methoxy-2-methyl-imidazo[1,2-a]pyridine (86 mg, 333.72 μmol) in Dioxane (5 mL) were added Cs₂CO₃ (181 mg, 556.20 μmol), BrettPhos Pd G3 (25 mg, 27.81 μmol) at room temperature under nitrogen atmosphere. The resulting mixture was stirred for 4 h at 80° C. under nitrogen atmosphere. After filtration, the filtrate was concentrated under reduced pressure. The residue was purified by silica gel column chromatography, eluted with PE/EA (1:10) to afford tert-butyl N-cyclopropyl-N-[1-[5-fluoro-7-[(8-fluoro-7-methoxy-2-methyl-imidazo[1,2-a]pyridin-6-yl)carbamoyl]-2-methyl-indazol-4-yl]-4-piperidyl]carbamate (0.13 g, 213.23 μmol, 77% yield) as a solid. LCMS (ES, m/z): 610 [M+H]⁺.

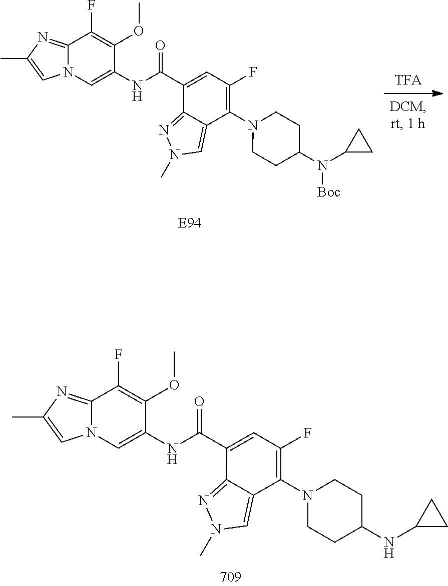 [SMILES: COc1c(NC(=O)c2cc(F)c(N3CCC(N(C(=O)OC(C)(C)C)C4CC4)CC3)c3cn(C)nc23)cn2cc(C)nc2c1F ; COc1c(NC(=O)c2cc(F)c(N3CCC(NC4CC4)CC3)c3cn(C)nc23)cn2cc(C)nc2c1F]

To a solution of tert-butyl N-cyclopropyl-N-[1-[5-fluoro-7-[(8-fluoro-7-methoxy-2-methyl-imidazo[1,2-a]pyridin-6-yl)carbamoyl]-2-methyl-indazol-4-yl]-4-piperidyl]carbamate (0.13 g, 213.23 μmol) in DCM (1 mL) was added TFA (0.4 mL). The reaction was stirred for 1 h at rt. The resulting mixture was concentrated under reduced pressure and adjusted to pH 8 with saturated NaHCO₃ (aq.). After filtration, the filtrate was concentrated under reduced pressure. The residue was purified by reversed-phase flash chromatography with the following conditions (column, C18 silica gel; mobile phase, CH₃CN in water (0.1% NH₃·H₂O), 43% to 80% gradient in 10 min; detector, UV 254 nm) to afford 4-[4-(cyclopropylamino)-1-piperidyl]-5-fluoro-N-(8-fluoro-7-methoxy-2-methyl-imidazo[1,2-a]pyridin-6-yl)-2-methyl-indazole-7-carboxamide (0.023 g, 45.14 μmol, 21% yield) as a solid. LCMS (ES, m/z): 510 [M+H]⁺. ¹H NMR (400 MHz, DMSO-d₆) δ 11.47 (s, 1H), 9.46 (d, J=1.2 Hz, 1H), 8.82 (s, 1H), 7.86-7.77 (m, 2H), 4.30 (s, 3H), 4.21 (d, J=1.9 Hz, 3H), 3.76 (d, J=12.5 Hz, 2H), 3.25 (s, 2H), 2.76 (s, 1H), 2.32 (s, 3H), 2.17-2.11 (m, 1H), 2.00 (d, J=12.6 Hz, 2H), 1.51 (d, J=10.9 Hz, 1H), 1.46 (d, J=10.6 Hz, 1H), 0.45-0.36 (m, 2H), 0.28-0.21 (m, 2H).

### Example 241: Synthesis of Compound 710

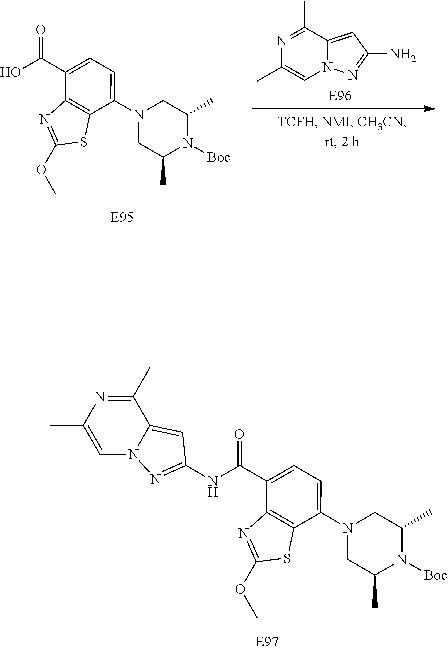

To a stirred mixture of 7-[(3S,5S)-4-tert-butoxycarbonyl-3,5-dimethyl-piperazin-1-yl]-2-methoxy-1,3-benzothiazole-4-carboxylic acid (60 mg, 142.35 μmol) and 4,6-dimethylpyrazolo[1,5-a]pyrazin-2-amine (27.7 mg, 170.81 μmol) in CH₃CN (2 mL) were added NMI (46.7 mg, 569.38 μmol) and TCFH (59.9 mg, 213.52 μmol) at room temperature. The resulting mixture was stirred for 2 h at rt. The resulting mixture was diluted with water (10 mL). The precipitated solids were collected by filtration and washed with water (2×6 mL). The resulting solid was dried under infrared light. This resulted in tert-butyl (2S,6S)-4-[4-[(4,6-dimethylpyrazolo[1,5-a]pyrazin-2-yl)carbamoyl]-2-methoxy-1,3-benzothiazol-7-yl]-2,6-dimethyl-piperazine-1-carboxylate (50 mg, 88.39 μmol, 62% yield) as a solid. LCMS (ES, m/z): 566 [M+H]⁺.

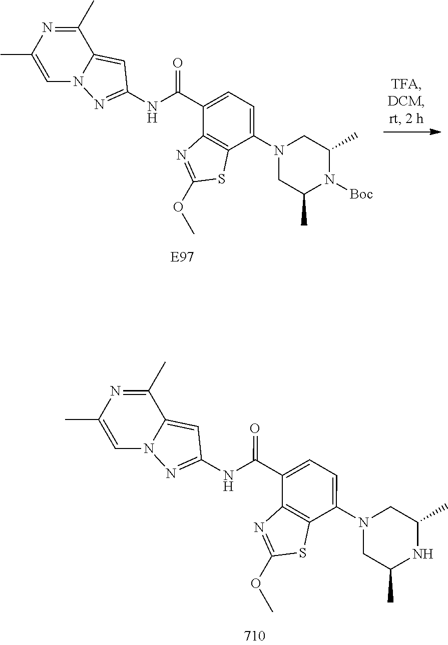

To a stirred mixture of tert-butyl (2S,6S)-4-[4-[(4,6-dimethylpyrazolo[1,5-a]pyrazin-2-yl)carbamoyl]-2-methoxy-1,3-benzothiazol-7-yl]-2,6-dimethyl-piperazine-1-carboxylate (45 mg, 79.55 μmol) in DCM (1 mL)) was added TFA (1 mL) dropwise at room temperature. The resulting mixture was stirred for 2 h at room temperature. The resulting mixture was concentrated under reduced pressure. The crude product was purified by Prep-HPLC with the following conditions (column, C18 silica gel, XBridge, 19×150 mm; mobile phase, MeCN in water (0.05% NH₃·H₂O), 45% to 90% gradient in 14 min; detector, UV 254 nm) to afford 7-[(3S,5S)-3,5-dimethylpiperazin-1-yl]-N-(4,6-dimethylpyrazolo[1,5-a]pyrazin-2-yl)-2-methoxy-1,3-benzothiazole-4-carboxamide (26 mg, 55.85 μmol, 70% yield) as a solid. LCMS (ES, m/z): 466 [M+H]⁺. ¹H NMR (400 MHz, DMSO-d₆) δ 12.12 (s, 1H), 8.39 (s, 1H), 8.18 (d, J=8.6 Hz, 1H), 7.26 (s, 1H), 7.08 (d, J=8.6 Hz, 1H), 4.38 (s, 3H), 3.23 (d, J=9.1 Hz, 2H), 3.14 (dd, J=11.5, 3.1 Hz, 2H), 2.94 (dd, J=11.5, 6.1 Hz, 2H), 2.67 (s, 3H), 2.40 (s, 3H), 1.17 (d, J=6.4 Hz, 6H).

### Example 242: Synthesis of Compound 711

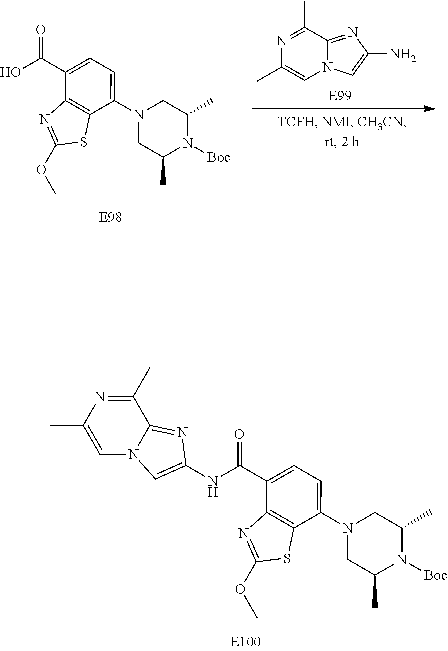

To a stirred mixture of 7-[(3S,5S)-4-tert-butoxycarbonyl-3,5-dimethyl-piperazin-1-yl]-2-methoxy-1,3-benzothiazole-4-carboxylic acid (60 mg, 142.35 μmol) and 6,8-dimethylimidazo[1,2-a]pyrazin-2-amine (27.7 mg, 170.81 μmol) in CH₃CN (2 mL) were added TCFH (59.9 mg, 213.52 μmol) and NMI (46.7 mg, 569.38 μmol) at room temperature. The resulting mixture was stirred for 2 h at rt. The resulting mixture was diluted with water (5 mL). The precipitated solids were collected by filtration and washed with water (2×6 mL). The resulting solid was dried under infrared light. This resulted in tert-butyl (2S,6S)-4-[4-[(6,8-dimethylimidazo[1,2-a]pyrazin-2-yl)carbamoyl]-2-methoxy-1,3-benzothiazol-7-yl]-2,6-dimethyl-piperazine-1-carboxylate (45 mg, 79.55 μmol, 56% yield) as a solid. LCMS (ES, m/z): 566 [M+H]⁺

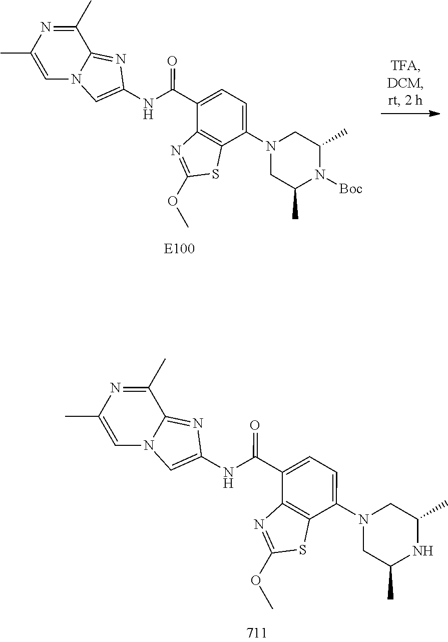

To a stirred mixture of tert-butyl (2S,6S)-4-[4-[(6,8-dimethylimidazo[1,2-a]pyrazin-2-yl)carbamoyl]-2-methoxy-1,3-benzothiazol-7-yl]-2,6-dimethyl-piperazine-1-carboxylate (40.0 mg, 70.71 μmol) in DCM (0.5 mL) was added TFA (0.5 mL) dropwise at room temperature. The resulting mixture was stirred for 2 h at room temperature. The resulting mixture was concentrated under reduced pressure. The crude product was purified by Prep-HPLC with the following conditions (column, C18 silica gel, XBridge, 19×150 mm; mobile phase, MeCN in water (0.05% NH₃·H₂O), 45% to 90% gradient in 14 min; detector, UV 254 nm) to afford N-(6,8-dimethylimidazo[1,2-a]pyrazin-2-yl)-7-[(3S,5S)-3,5-dimethylpiperazin-1-yl]-2-methoxy-1,3-benzothiazole-4-carboxamide (21.6 mg, 46.39 μmol, 66% yield) as a solid. LCMS (ES, m/z): 466 [M+H]⁺. ¹H NMR (400 MHz, DMSO-d₆) δ 12.01 (s, 1H), 8.31 (d, J=2.8 Hz, 2H), 8.17 (d, J=8.5 Hz, 1H), 7.06 (d, J=8.6 Hz, 1H), 4.40 (s, 3H), 3.27-3.18 (m, 2H), 3.18-3.07 (m, 2H), 2.93 (dd, J=11.3, 6.1 Hz, 2H), 2.67 (s, 3H), 2.37 (s, 3H), 1.17 (d, J=6.4 Hz, 6H).

### Example 243: Synthesis of Compound 712

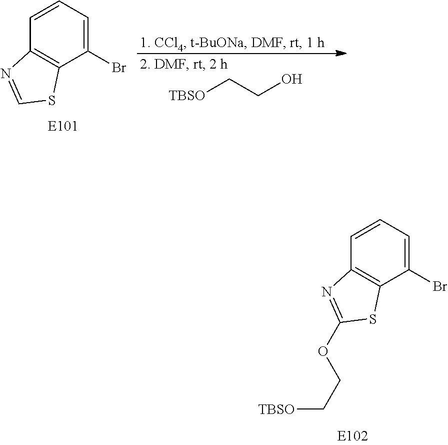 [SMILES: Brc1cccc2ncsc12 ; CC(C)(C)[Si](C)(C)OCCO ; CC(C)(C)[Si](C)(C)OCCOc1nc2cccc(Br)c2s1]

A solution of 2-[tert-butyl(dimethyl)silyl]oxyethanol (2.64 g, 14.95 mmol) in DMF (16 mL) was treated with NaH (60% dispersion in oil) (358.7 mg, 14.95 mmol) for 1 h at 0° C. under nitrogen atmosphere followed by the addition of CCl₄ (681.6 mg, 4.48 mmol) and 7-bromo-1,3-benzothiazole (800 mg, 3.74 mmol) at 0° C. The resulting mixture was stirred for 2 h at room temperature. The reaction was quenched with sat. NH₄Cl (aq.) at 0° C. The resulting mixture was extracted with EA (2×30 mL). The combined organic layers were washed with brine (1×30 mL), dried over anhydrous Na₂SO₄. After filtration, the filtrate was concentrated under reduced pressure. The residue was purified by silica gel column chromatography, eluted with PE/EA (4:1) to afford 2-[(7-bromo-1,3-benzothiazol-2-yl)oxy]ethoxy-tert-butyl-dimethyl-silane (980 mg, 1.29 mmol, 68% yield) as a solid. LCMS (ES, m/z): 388 [M+H]⁺.

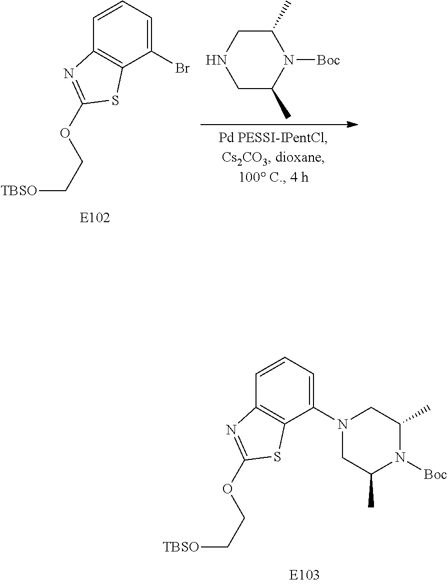

To a stirred mixture of 2-[(7-bromo-1,3-benzothiazol-2-yl)oxy]ethoxy-tert-butyl-dimethyl-silane (980 mg, 2.52 mmol) and tert-butyl (2S,6S)-2,6-dimethylpiperazine-1-carboxylate (702.9 mg, 3.28 mmol) in dioxane (15 mL) were added Cs₂CO₃ (2.47 g, 7.57 mmol) and Pd PESSI-IPentCl (211.9 mg, 252.32 μmol) at room temperature under nitrogen atmosphere. The resulting mixture was stirred for 4 h at 100° C. under nitrogen atmosphere. The mixture was allowed to cool down to room temperature. The mixture was concentrated off under reduce pressure. The residue was purified by silica gel column chromatography, eluted with PE/EA (5:1) to afford tert-butyl (2S,6S)-4-[2-[2-[tert-butyl(dimethyl)silyl]oxyethoxy]-1,3-benzothiazol-7-yl]-2,6-dimethyl-piperazine-1-carboxylate (410 mg, 785.76 μmol, 31% yield) as a solid. LCMS (ES, m/z): 522 [M+H]⁺

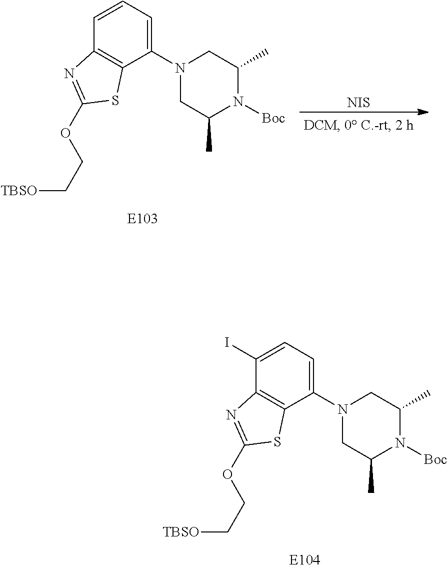 [SMILES: C[C@H]1CN(c2cccc3nc(OCCO[Si](C)(C)C(C)(C)C)sc23)C[C@H](C)N1C(=O)OC(C)(C)C ; "NIS" ; C[C@H]1CN(c2ccc(I)c3nc(OCCO[Si](C)(C)C(C)(C)C)sc23)C[C@H](C)N1C(=O)OC(C)(C)C]

A solution of tert-butyl (2S,6S)-4-[2-[2-[tert-butyl(dimethyl)silyl]oxyethoxy]-1,3-benzothiazol-7-yl]-2,6-dimethyl-piperazine-1-carboxylate (410 mg, 785.76 μmol) in DCM (5 mL) was treated with NIS (176.7 mg, 785.76 μmol) at 0° C. under nitrogen atmosphere. The resulting mixture was stirred for 2 h at room temperature under nitrogen atmosphere. The resulting mixture was diluted with water (8 mL). The resulting mixture was extracted with EA (2×10 mL). The combined organic layers were washed with brine (1×10 mL), dried over anhydrous Na₂SO₄. After filtration, the filtrate was concentrated under reduced pressure to afford tert-butyl (2S,6S)-4-[2-[2-[tert-butyl(dimethyl)silyl]oxyethoxy]-4-iodo-1,3-benzothiazol-7-yl]-2,6-dimethyl-piperazine-1-carboxylate (403 mg, 622.22 μmol, 79% yield) as a solid. LCMS (ES, m/z): 648 [M+H]⁺.

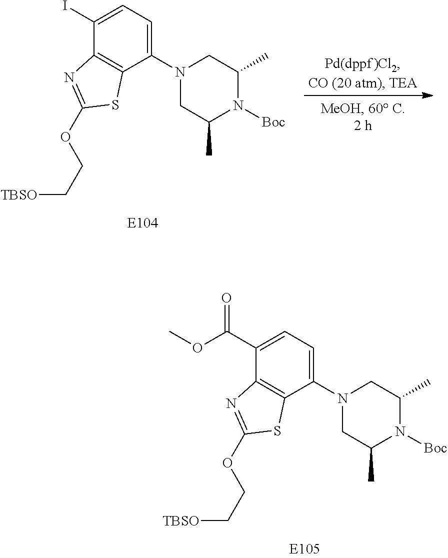 [SMILES: C[C@H]1CN(c2ccc(I)c3nc(OCCO[Si](C)(C)C(C)(C)C)sc23)C[C@H](C)N1C(=O)OC(C)(C)C ; "Pd(dppf)Cl2, CO (20 atm), TEA" ; COC(=O)c1ccc(N2C[C@H](C)N(C(=O)OC(C)(C)C)[C@@H](C)C2)c2sc(OCCO[Si](C)(C)C(C)(C)C)nc12]

To a solution of tert-butyl (2S,6S)-4-[2-[2-[tert-butyl(dimethyl)silyl]oxyethoxy]-4-iodo-1,3-benzothiazol-7-yl]-2,6-dimethyl-piperazine-1-carboxylate (400 mg, 617.59 μmol) and TEA (187.4 mg, 1.85 mmol) in MeOH (10 mL) was added Pd(dppf)Cl₂ (45.1 mg, 61.76 μmol) in a pressure tank. The mixture was purged with nitrogen and then was pressurized to 20 atm with carbon monoxide at 60° C. for 2 h. The reaction mixture was cooled to room temperature and filtered to remove insoluble solids. The resulting mixture was concentrated under reduced pressure. The residue was purified by silica gel column chromatography, eluted with PE/EA (3:1) to afford methyl 7-[(3S,5S)-4-tert-butoxycarbonyl-3,5-dimethyl-piperazin-1-yl]-2-[2-[tert-butyl(dimethyl)silyl]oxyethoxy]-1,3-benzothiazole-4-carboxylate (341 mg, 588.11 μmol, 95% yield) as a solid. LCMS (ES, m/z): 580 [M+H]⁺.

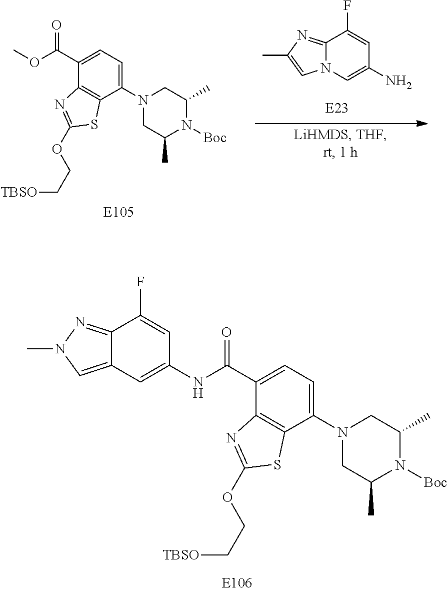 [SMILES: COC(=O)c1ccc(N2C[C@H](C)N(C(=O)OC(C)(C)C)[C@@H](C)C2)c2sc(OCCO[Si](C)(C)C(C)(C)C)nc12 ; Cc1cn2cc(N)cc(F)c2n1 ; C[C@H]1CN(c2ccc(C(=O)Nc3cc(F)c4nn(C)cc4c3)c3nc(OCCO[Si](C)(C)C(C)(C)C)sc23)C[C@H](C)N1C(=O)OC(C)(C)C]

To a stirred solution of 7-fluoro-2-methyl-indazol-5-amine (31.3 mg, 189.71 μmol) and methyl 7-[(3S,5S)-4-tert-butoxycarbonyl-3,5-dimethyl-piperazin-1-yl]-2-[2-[tert-butyl(dimethyl)silyl]oxyethoxy]-1,3-benzothiazole-4-carboxylate (100 mg, 172.47 μmol) in THF (2 mL) was added LiHMDS (115.4 mg, 689.87 μmol) dropwise at 0° C. under N₂ atmosphere. The resulting mixture was stirred for 1 h at rt under N₂ atmosphere. The resulting mixture was diluted with NH₄Cl aq. The resulting mixture was extracted with EA (3×10 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (1:3) to afford tert-butyl (2S,6S)-4-[2-[2-[tert-butyl(dimethyl)silyl]oxyethoxy]-4-[(7-fluoro-2-methyl-indazol-5-yl)carbamoyl]-1,3-benzothiazol-7-yl]-2,6-dimethyl-piperazine-1-carboxylate (83 mg, 116.42 μmol, 68% yield) as a gum. LCMS (ES, m/z): 713 [M+H]⁺.

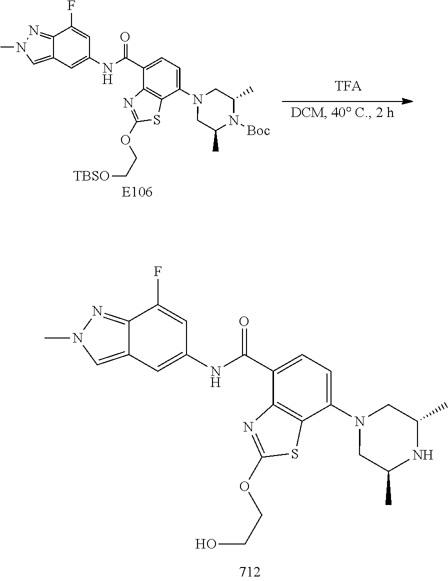

To a stirred mixture of tert-butyl (2S,6S)-4-[2-[2-[tert-butyl(dimethyl)silyl]oxyethoxy]-4-[(7-fluoro-2-methyl-indazol-5-yl)carbamoyl]-1,3-benzothiazol-7-yl]-2,6-dimethyl-piperazine-1-carboxylate (70 mg, 98.18 μmol) in DCM (1 mL) was added TFA (1 mL) dropwise at room temperature. The resulting mixture was stirred for 2 h at 40° C. The resulting mixture was concentrated under reduced pressure. The crude product was purified by Prep-HPLC with the following conditions (column, C18 silica gel, XBridge, 19×150 mm; mobile phase, MeCN in water (0.05% NH₃·H₂O), 35% to 75% gradient in 7 min; detector, UV 254 nm) to afford 7-[(3S,5S)-3,5-dimethylpiperazin-1-yl]-N-(7-fluoro-2-methyl-indazol-5-yl)-2-(2-hydroxyethoxy)-1,3-benzothiazole-4-carboxamide (25 mg, 50.14 μmol, 51% yield) as a solid. LCMS (ES, m/z): 499 [M+H]⁺. ¹H NMR (400 MHz, DMSO-d₆) δ 11.34 (s, 1H), 8.44 (d, J=2.8 Hz, 1H), 8.12 (d, J=8.5 Hz, 1H), 8.09 (d, J=1.6 Hz, 1H), 7.35 (d, J=13.1 Hz, 1H), 7.05 (d, J=8.5 Hz, 1H), 5.12 (s, 1H), 4.80 (t, J=4.7 Hz, 2H), 4.20 (s, 3H), 4.03-3.79 (m, 2H), 3.24 (q, J=6.1 Hz, 2H), 3.11 (dd, J=11.6, 3.2 Hz, 2H), 2.93 (dd, J=11.5, 6.1 Hz, 2H), 1.18 (d, J=6.3 Hz, 6H).

### Example 244: Synthesis of Compound 713

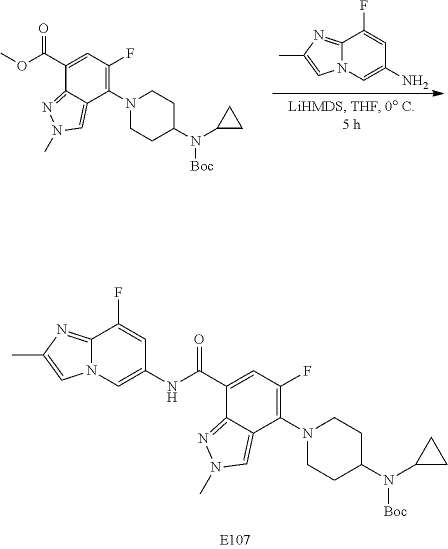 [SMILES: COC(=O)c1cc(F)c(N2CCC(N(C(=O)OC(C)(C)C)C3CC3)CC2)c2cn(C)nc12 ; Cc1cn2cc(N)cc(F)c2n1 ; Cc1cn2cc(NC(=O)c3cc(F)c(N4CCC(N(C(=O)OC(C)(C)C)C5CC5)CC4)c4cn(C)nc34)cc(F)c2n1]

To a stirred mixture of methyl 4-[4-[tert-butoxycarbonyl(cyclopropyl)amino]-1-piperidyl]-5-fluoro-2-methyl-indazole-7-carboxylate (0.15 g, 335.94 μmol) and 8-fluoro-2-methyl-imidazo[1,2-a]pyridin-6-amine (66 mg, 403.12 μmol) in THF (3 mL) were added LiHMDS (56 mg, 335.94 μmol) at room temperature under nitrogen atmosphere. The resulting mixture was stirred for 5 h at 0° C. under nitrogen atmosphere. The resulting mixture was diluted with water (5 mL). The resulting mixture was extracted with EA (3×5 mL). The combined organic layers were washed with water (1×20 mL), brine (1×20 mL), dried over anhydrous Na₂SO₄. After filtration, the filtrate was concentrated under reduced pressure. The residue was purified by silica gel column chromatography, eluted with PE/EA (1:8) to afford tert-butyl cyclopropyl(1-(5-fluoro-7-((8-fluoro-2-methylimidazo[1,2-a]pyridin-6-yl)carbamoyl)-2-methyl-2H-indazol-4-yl)piperidin-4-yl)carbamate (0.145 g, 250.16 μmol, 74% yield) as a solid. LCMS (ES, m/z): 580 [M+H]⁺.

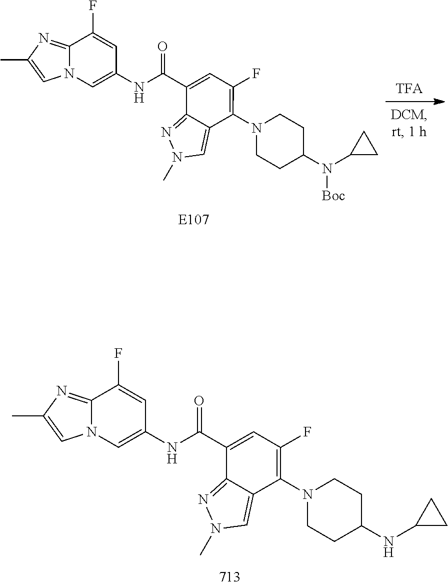 [SMILES: Cc1cn2cc(NC(=O)c3cc(F)c(N4CCC(N(C(=O)OC(C)(C)C)C5CC5)CC4)c4cn(C)nc34)cc(F)c2n1 ; Cc1cn2cc(NC(=O)c3cc(F)c(N4CCC(NC5CC5)CC4)c4cn(C)nc34)cc(F)c2n1]

To a solution of tert-butyl N-cyclopropyl-N-[1-[5-fluoro-7-[(8-fluoro-2-methyl-imidazo[1,2-a]pyridin-6-yl)carbamoyl]-2-methyl-indazol-4-yl]-4-piperidyl]carbamate (0.145 g, 250.16 μmol) in DCM (1 mL) was added TFA (0.4 mL). The reaction was stirred for 1 h at rt. The resulting mixture was concentrated under reduced pressure and adjusted to pH 8 with saturated NaHCO₃ (aq.). After filtration, the filtrate was concentrated under reduced pressure. The residue was purified by reversed-phase flash chromatography with the following conditions (column, C18 silica gel; mobile phase, CH₃CN in water (0.1% NH₃·H₂O), 30% to 70% gradient in 10 min; detector, UV 254 nm) to afford 4-[4-(cyclopropylamino)-1-piperidyl]-5-fluoro-N-(8-fluoro-2-methyl-imidazo[1,2-a]pyridin-6-yl)-2-methyl-indazole-7-carboxamide (0.04 g, 83.42 μmol, 33% yield) as a solid. LCMS (ES, m/z): 480 [M+H]⁺. ¹H NMR (400 MHz, DMSO-d₆) δ 11.01 (s, 1H), 9.21 (s, 1H), 8.81 (s, 1H), 7.92 (d, J=3.0 Hz, 1H), 7.80 (d, J=14.5 Hz, 1H), 7.35 (d, J=12.3 Hz, 1H), 4.31 (s, 3H), 3.76 (d, J=12.6 Hz, 2H), 3.25 (t, J=11.6 Hz, 3H), 2.77 (s, 1H), 2.36 (s, 3H), 2.18 (s, 1H), 2.00 (d, J=12.6 Hz, 2H), 1.49 (d, J=11.3 Hz, 2H), 0.41 (dd, J=6.5, 4.3 Hz, 2H), 0.26 (d, J=3.6 Hz, 2H).

### Example 245: Synthesis of Compound 714

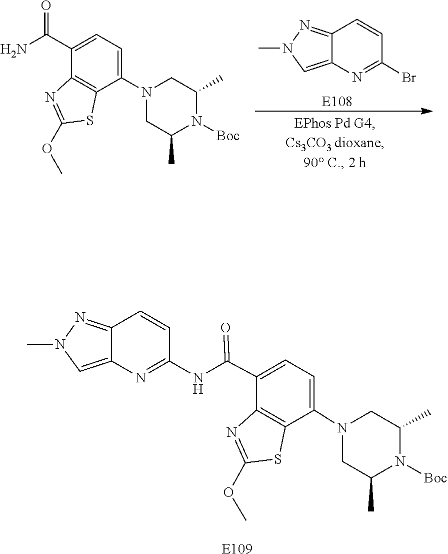

To a stirred mixture of tert-butyl (2S,6S)-4-(4-carbamoyl-2-methoxy-1,3-benzothiazol-7-yl)-2,6-dimethyl-piperazine-1-carboxylate (40 mg, 95.12 μmol) and 5-bromo-2-methyl-pyrazolo[4,3-b]pyridine (24.2 mg, 114.14 μmol) in Dioxane (1 mL) were added Cs₂CO₃ (93.0 mg, 285.36 μmol) and EPhos Pd G4 (8.7 mg, 9.51 μmol) at room temperature under nitrogen atmosphere. The resulting mixture was stirred for 2 h at 90° C. under nitrogen atmosphere. The resulting mixture was concentrated under reduced pressure. The residue was purified by silica gel column chromatography, eluted with DCM/MeOH (10:1) to afford tert-butyl (2S,6S)-4-[2-methoxy-4-[(2-methylpyrazolo[4,3-b]pyridin-5-yl)carbamoyl]-1,3-benzothiazol-7-yl]-2,6-dimethyl-piperazine-1-carboxylate (44 mg, 79.76 μmol, 84% yield) as a solid. LCMS (ES, m/z): 552 [M+H]⁺.

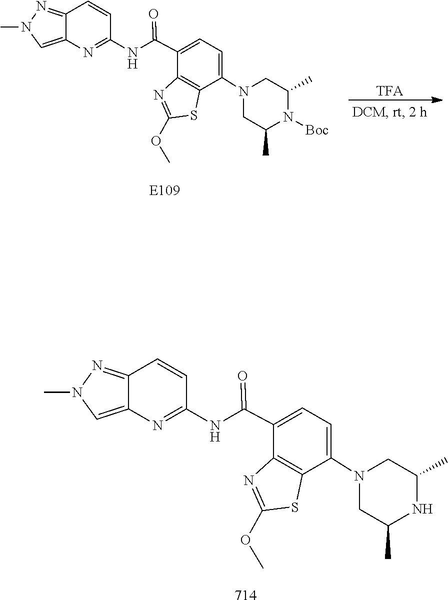

To a stirred mixture of tert-butyl (2S,6S)-4-[2-methoxy-4-[(2-methylpyrazolo[4,3-b]pyridin-5-yl)carbamoyl]-1,3-benzothiazol-7-yl]-2,6-dimethyl-piperazine-1-carboxylate (44 mg, 79.76 μmol) in DCM (1.00 mL) was added TFA (0.3 mL) dropwise at room temperature. The resulting mixture was stirred for 2 h at room temperature. The resulting mixture were concentrated under reduced pressure. The crude product was purified by Prep-HPLC with the following conditions (column, C18 silica gel, XBridge, 19×150 mm; mobile phase, MeCN in water (0.05% NH₃H₂O), 40% to 90% gradient in 7 min; detector, UV 254 nm) to afford 7-[(3S,5S)-3,5-dimethylpiperazin-1-yl]-2-methoxy-N-(2-methylpyrazolo[4,3-b]pyridin-5-yl)-1,3-benzothiazole-4-carboxamide (8.6 mg, 19.05 μmol, 24% yield) as a solid. LCMS (ES, m/z): 452 [M+H]⁺. ¹H NMR (400 MHz, DMSO-d₆) δ 12.12 (s, 1H), 8.44-8.35 (m, 2H), 8.17 (dd, J=18.7, 8.9 Hz, 2H), 7.07 (d, J=8.6 Hz, 1H), 4.40 (s, 3H), 4.19 (s, 3H), 3.24 (tt, J=9.2, 4.7 Hz, 2H), 3.14 (dd, J=11.6, 3.2 Hz, 2H), 2.95 (dd, J=11.5, 6.1 Hz, 2H), 1.18 (d, J=6.4 Hz, 6H).

### Example 247: Synthesis of Compound 716

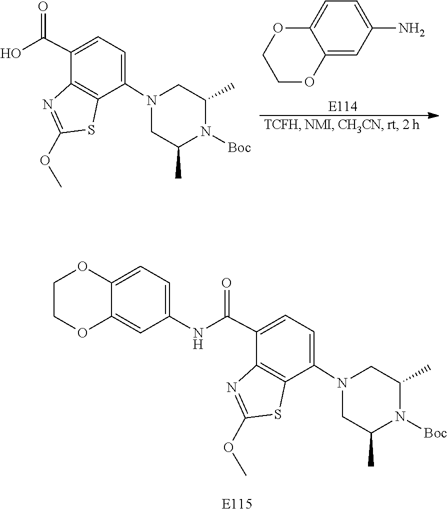

To a stirred mixture of 7-[(3S,5S)-4-tert-butoxycarbonyl-3,5-dimethyl-piperazin-1-yl]-2-methoxy-1,3-benzothiazole-4-carboxylic acid (0.05 g, 118.62 μmol) and 2,3-dihydro-1,4-benzodioxin-6-amine (21.5 mg, 142.35 μmol, 17.48 μL) in MeCN (1 mL) were added NMI (29.2 mg, 355.86 μmol), TCFH (49.9 mg, 177.93 μmol) at room temperature. The resulting mixture was stirred for 2 h at room temperature. The resulting mixture was diluted with water (3 mL). The resulting mixture was extracted with EA (3×3 mL). The combined organic layers were washed with water (1×10 mL), brine (1×10 mL), dried over anhydrous Na₂SO₄. After filtration, the filtrate was concentrated under reduced pressure to afford tert-butyl (2S,6S)-4-[4-(2,3-dihydro-1,4-benzodioxin-6-ylcarbamoyl)-2-methoxy-1,3-benzothiazol-7-yl]-2,6-dimethyl-piperazine-1-carboxylate (0.055 g, 99.16 μmol, 84% yield) as a solid. LCMS (ES, m/z): 555 [M+H]⁺.

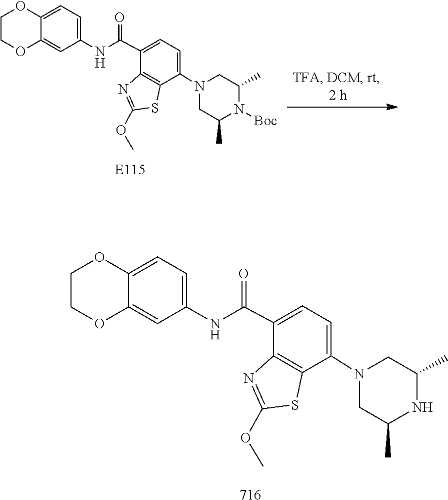

To a solution of tert-butyl (2S,6S)-4-[4-(2,3-dihydro-1,4-benzodioxin-6-ylcarbamoyl)-2-methoxy-1,3-benzothiazol-7-yl]-2,6-dimethyl-piperazine-1-carboxylate (0.55 g, 991.60 μmol) in DCM (1 mL) was added TFA (0.4 mL). The reaction was stirred for 1 h at rt. The resulting mixture was concentrated under reduced pressure and adjusted to pH 8 with saturated NaHCO₃ (aq.). After filtration, the filtrate was concentrated under reduced pressure. The residue was purified by reversed-phase flash chromatography with the following conditions (column, C18 silica gel; mobile phase, CH₃CN in water (0.1% NH₃·H₂O), 45% to 75% gradient in 10 min; detector, UV 254 nm) to afford N-(2,3-dihydro-1,4-benzodioxin-6-yl)-7-[(3S,5S)-3,5-dimethylpiperazin-1-yl]-2-methoxy-1,3-benzothiazole-4-carboxamide (0.25 g, 550.01 μmol, 55% yield) as a solid. LCMS (ES, m/z): 455 [M+H]⁺. ¹H NMR (400 MHz, DMSO-d₆) δ 11.31 (s, 1H), 8.10 (d, J=8.5 Hz, 1H), 7.48 (d, J=2.5 Hz, 1H), 7.11 (dd, J=8.8, 2.6 Hz, 1H), 7.04 (d, J=8.6 Hz, 1H), 6.86 (d, J=8.7 Hz, 1H), 4.36 (s, 3H), 4.25 (q, J=5.2 Hz, 4H), 3.26 (s, 3H), 3.11 (d, J=11.3 Hz, 2H), 2.92 (dd, J=11.6, 6.1 Hz, 2H), 1.18 (d, J=6.4 Hz, 6H).

### Example 248: Synthesis of Compound 717

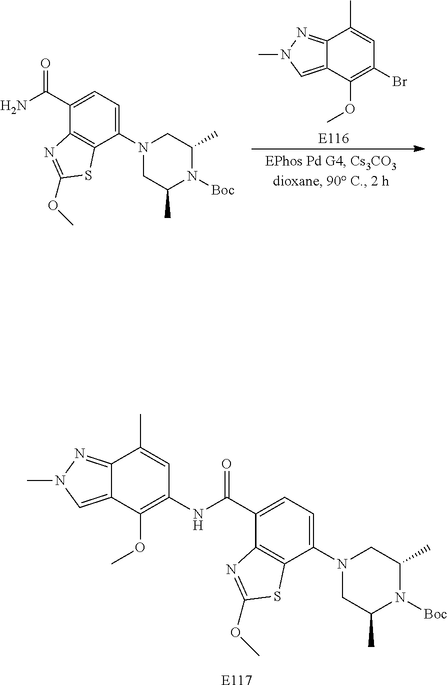

To a stirred mixture of tert-butyl (2S,6S)-4-(4-carbamoyl-2-methoxy-1,3-benzothiazol-7-yl)-2,6-dimethyl-piperazine-1-carboxylate (40 mg, 95.12 μmol) and 5-bromo-4-methoxy-2,7-dimethyl-indazole (29.1 mg, 114.14 μmol) in Dioxane (1 mL) were added Cs₂CO₃ (93.0 mg, 285.36 μmol) and EPhos Pd G4 (8.7 mg, 9.51 μmol) at room temperature under nitrogen atmosphere. The resulting mixture was stirred for 2 h at 90° C. under nitrogen atmosphere. The resulting mixture was concentrated under reduced pressure. The residue was purified by silica gel column chromatography, eluted with DCM/MeOH (10:1) to afford tert-butyl (2S,6S)-4-[2-methoxy-4-[(4-methoxy-2,7-dimethyl-indazol-5-yl)carbamoyl]-1,3-benzothiazol-7-yl]-2,6-dimethyl-piperazine-1-carboxylate (40 mg, 67.26 μmol, 71% yield) as a solid. LCMS (ES, m/z): 595 [M+H]⁺.

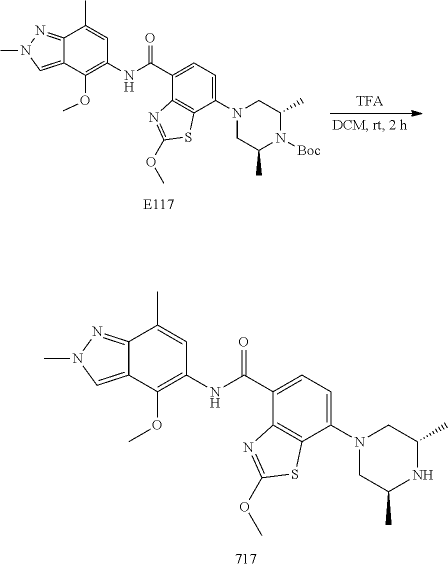

To a stirred mixture of tert-butyl (2S,6S)-4-[2-methoxy-4-[(4-methoxy-2,7-dimethyl-indazol-5-yl)carbamoyl]-1,3-benzothiazol-7-yl]-2,6-dimethyl-piperazine-1-carboxylate (40 mg, 67.26 μmol) in DCM (1 mL) was added TFA (0.25 mL) dropwise at room temperature. The resulting mixture was stirred for 2 h at room temperature. The resulting mixture were concentrated under reduced pressure. The crude product was purified by Prep-HPLC with the following conditions (column, C18 silica gel, XBridge, 19×150 mm; mobile phase, MeCN in water (0.05% NH₃H₂O), 40% to 90% gradient in 7 min; detector, UV 254 nm) to afford 7-[(3S,5S)-3,5-dimethylpiperazin-1-yl]-2-methoxy-N-(4-methoxy-2,7-dimethyl-indazol-5-yl)-1,3-benzothiazole-4-carboxamide (10.8 mg, 21.84 μmol, 32% yield) as a solid. LCMS (ES, m/z): 495 [M+H]⁺. ¹H NMR (400 MHz, DMSO-d₆) δ 11.39 (s, 1H), 8.58 (s, 1H), 8.19 (d, J=8.5 Hz, 1H), 8.10 (s, 1H), 7.06 (d, J=8.5 Hz, 1H), 4.39 (s, 3H), 4.17 (s, 3H), 4.06 (s, 3H), 3.24 (tt, J=8.7, 4.3 Hz, 2H), 3.11 (dd, J=11.5, 3.1 Hz, 2H), 2.92 (dd, J=11.4, 6.0 Hz, 2H), 2.47 (s, 3H), 1.18 (d, J=6.4 Hz, 6H).

### Example 249: Synthesis of Compound 718

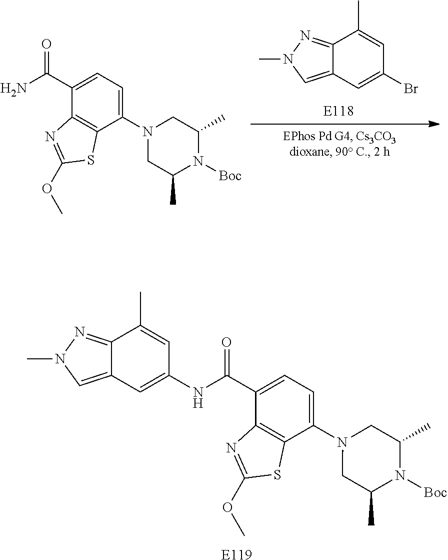

To a stirred mixture of tert-butyl (2S,6S)-4-(4-carbamoyl-2-methoxy-1,3-benzothiazol-7-yl)-2,6-dimethyl-piperazine-1-carboxylate (40 mg, 95.12 μmol) and 5-bromo-2,7-dimethyl-indazole (25.7 mg, 114.14 μmol) in Dioxane (1 mL) were added Cs₂CO₃ (93.0 mg, 285.36 μmol) and EPhos Pd G4 (8.7 mg, 9.51 μmol) at room temperature under nitrogen atmosphere. The resulting mixture was stirred for 2 h at 90° C. under nitrogen atmosphere. The resulting mixture was concentrated under reduced pressure. The residue was purified by silica gel column chromatography, eluted with DCM/MeOH (10:1) to afford tert-butyl (2S,6S)-4-[4-[(2,7-dimethylindazol-5-yl)carbamoyl]-2-methoxy-1,3-benzothiazol-7-yl]-2,6-dimethyl-piperazine-1-carboxylate (48 mg, 85.00 μmol, 89% yield) as a solid. LCMS (ES, m/z): 565 [M+H]⁺.

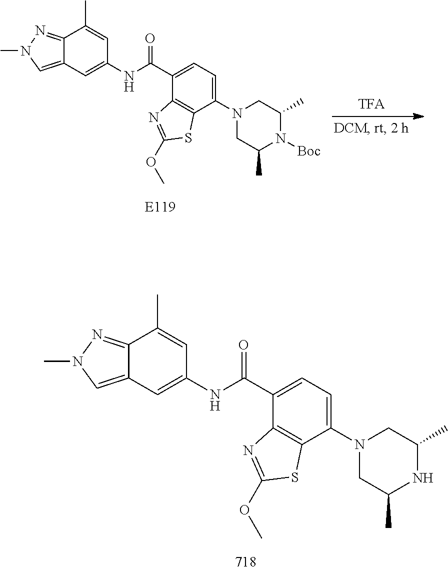

To a stirred mixture of tert-butyl (2S,6S)-4-[4-[(2,7-dimethylindazol-5-yl)carbamoyl]-2-methoxy-1,3-benzothiazol-7-yl]-2,6-dimethyl-piperazine-1-carboxylate (48 mg, 85.00 μmol) in DCM (1 mL) was added TFA (0.4 mL) dropwise at room temperature. The resulting mixture was stirred for 2 h at room temperature. The resulting mixture were concentrated under reduced pressure. The crude product was purified by Prep-HPLC with the following conditions (column, C18 silica gel, XBridge, 19×150 mm; mobile phase, MeCN in water (0.05% NH₃H₂O), 40% to 90% gradient in 7 min; detector, UV 254 nm) to afford N-(2,7-dimethylindazol-5-yl)-7-[(3S,5S)-3,5-dimethylpiperazin-1-yl]-2-methoxy-1,3-benzothiazole-4-carboxamide (13.1 mg, 28.20 μmol, 33% yield) as a solid. LCMS (ES, m/z): 665 [M+H]⁺. ¹H NMR (400 MHz, DMSO-d₆) δ 11.37 (s, 1H), 8.26 (s, 1H), 8.20 (s, 1H), 8.14 (d, J=8.4 Hz, 1H), 7.10 (s, 1H), 7.05 (d, J=8.6 Hz, 1H), 4.41 (s, 3H), 4.16 (s, 3H), 3.25 (td, J=6.5, 3.1 Hz, 2H), 3.11 (dd, J=11.7, 3.1 Hz, 2H), 2.92 (dd, J=11.5, 6.1 Hz, 2H), 1.18 (d, J=6.4 Hz, 6H).

### Example 250: Synthesis of Compound 719

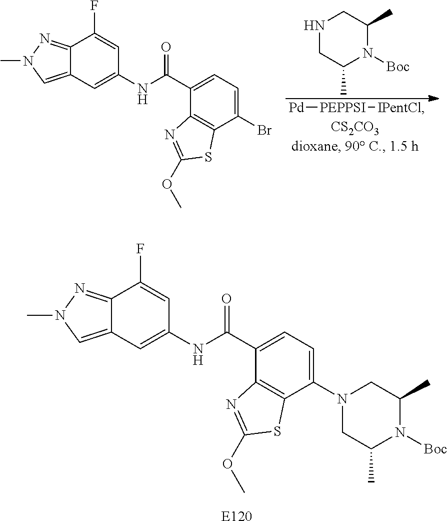

To a stirred mixture of 7-bromo-N-(7-fluoro-2-methyl-indazol-5-yl)-2-methoxy-1,3-benzothiazole-4-carboxamide (0.042 g, 96.49 μmol) and tert-butyl(2R,6R)-2,6-dimethylpiperazine-1-carboxylate (24.8 mg, 115.79 μmol) in Dioxane (1 mL) were added Cs₂CO₃ (62.8 mg, 192.98 μmol), Pd-PEPPSI™-IPent catalyst (7.6 mg, 9.65 μmol) at room temperature under nitrogen atmosphere. The resulting mixture was stirred for 1.5 h at 90° C. under nitrogen atmosphere. The mixture was allowed to cool down to room temperature. After filtration, the filtrate was concentrated under reduced pressure. The residue was purified by silica gel column chromatography, eluted with PE/EA (1:10) to afford tert-butyl(2R,6R)-4-[4-[(7-fluoro-2-methyl-indazol-5-yl)carbamoyl]-2-methoxy-1,3-benzothiazol-7-yl]-2,6-dimethyl-piperazine-1-carboxylate (0.039 g, 68.58 μmol, 71% yield) as a solid. LCMS (ES, m/z): 569 [M+H]⁺.

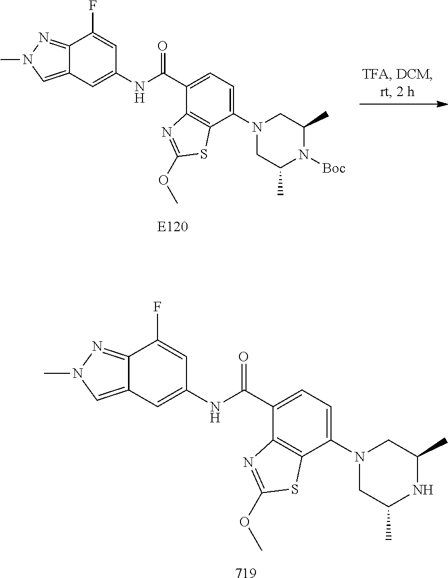

To a solution of tert-butyl(2R,6R)-4-[4-[(7-fluoro-2-methyl-indazol-5-yl)carbamoyl]-2-methoxy-1,3-benzothiazol-7-yl]-2,6-dimethyl-piperazine-1-carboxylate (0.039 g, 68.58 μmol) in DCM (0.5 mL) was added TFA (0.2 mL). The reaction was stirred for 2 h at rt. The resulting mixture was concentrated under reduced pressure and adjusted to pH 8 with saturated NaHCO₃ (aq.). The residue was purified by reversed-phase flash chromatography with the following conditions (column, C18 silica gel; mobile phase, CH₃CN in water (0.1% NH₃·H₂O), 45% to 70% gradient in 10 min; detector, UV 254 nm) to afford 7-[(3R,5R)-3,5-dimethylpiperazin-1-yl]-N-(7-fluoro-2-methyl-indazol-5-yl)-2-methoxy-1,3-benzothiazole-4-carboxamide (0.013 g, 27.75 μmol, 40% yield) as a solid. LCMS (ES, m/z): 469 [M+H]⁺. ¹H NMR (400 MHz, DMSO-d₆) δ 11.41 (s, 1H), 8.44 (d, J=2.8 Hz, 1H), 8.16-8.08 (m, 2H), 7.40-7.32 (m, 1H), 7.06 (d, J=8.6 Hz, 1H), 4.41 (s, 3H), 4.19 (s, 3H), 3.28-3.21 (m, 2H), 3.11 (dd, J=11.5, 3.0 Hz, 2H), 2.93 (dd, J=11.4, 6.1 Hz, 2H), 1.18 (d, J=6.4 Hz, 6H).

### Example 251: Synthesis of Compound 721

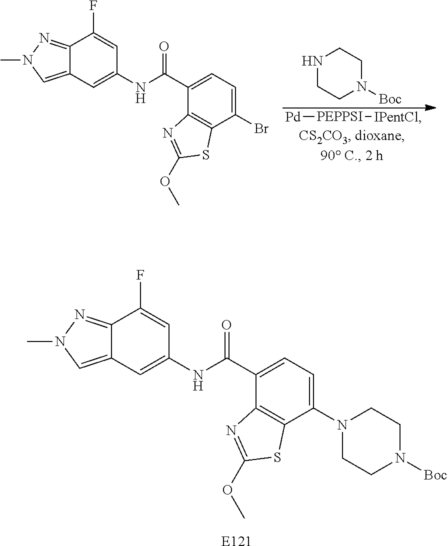

To a stirred mixture of 7-bromo-N-(7-fluoro-2-methyl-indazol-5-yl)-2-methoxy-1,3-benzothiazole-4-carboxamide (60 mg, 137.85 μmol) and tert-butyl piperazine-1-carboxylate (30.8 mg, 165.41 μmol) in dioxane (1.5 mL) were added Cs₂CO₃ (89.8 mg, 275.69 μmol) and Pd-PEPPSI-IPentCl (12.6 mg, 13.78 μmol) at room temperature under nitrogen atmosphere. The resulting mixture was stirred for 2 h at 90° C. under nitrogen atmosphere. The resulting mixture was concentrated under reduced pressure. The residue was purified by silica gel column chromatography, eluted with PE/EA (1:3) to afford tert-butyl 4-[4-[(7-fluoro-2-methyl-indazol-5-yl)carbamoyl]-2-methoxy-1,3-benzothiazol-7-yl]piperazine-1-carboxylate (65 mg, 120.23 μmol, 87.22% yield) as a solid. LCMS (ES, m/z): 541 [M+H]⁺.

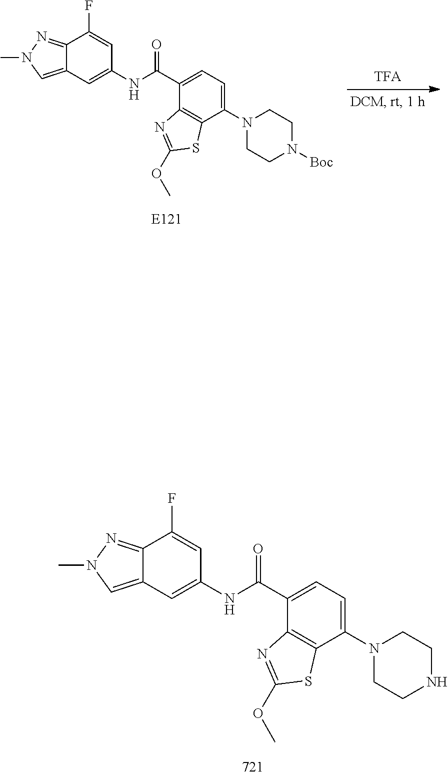

To a stirred solution of tert-butyl 4-[4-[(8-fluoro-2-methyl-imidazo[1,2-a]pyridin-6-yl)carbamoyl]-2-methoxy-1,3-benzothiazol-7-yl]piperazine-1-carboxylate (65 mg, 120.23 μmol) in DCM (2 mL) was added TFA (1 mL) at rt. The resulting mixture was stirred for 2 h. The resulting mixture was concentrated under reduced pressure. The crude product was purified by Prep-HPLC with the following conditions (SunFire Prep C18 OBD Column 19*150 mm, 5 Um 10 nm, mobile phase, MeCN in water (0.05% NH₄OH), 30% to 50% gradient in 7 min; detector, UV 254 nm) to afford N-(8-fluoro-2-methyl-imidazo[1,2-a]pyridin-6-yl)-2-methoxy-7-piperazin-1-yl-1,3-benzothiazole-4-carboxamide (7.2 mg, 16.35 μmol, 14% yield) as a solid. LCMS (ES, m/z): 441 [M+H]⁺. ¹H NMR (400 MHz, DMSO-d₆) δ 11.40 (s, 1H), 8.43 (d, J=2.8 Hz, 1H), 8.17-8.08 (m, 2H), 7.36 (d, J=13.1 Hz, 1H), 7.08 (d, J=8.5 Hz, 1H), 4.41 (s, 3H), 4.19 (s, 3H), 3.18-3.11 (m, 4H), 2.91 (t, J=4.8 Hz, 4H).

### Example 252: Synthesis of Compound 722

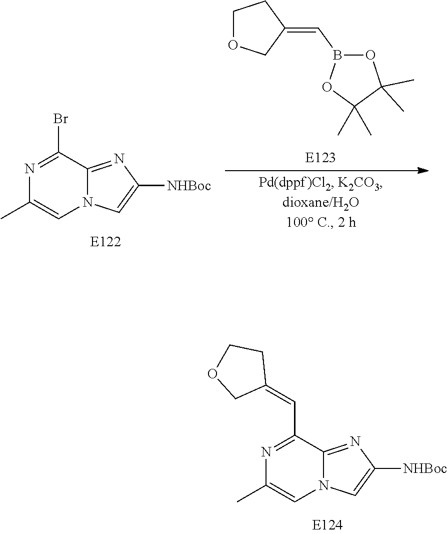 [SMILES: Cc1cn2cc(NC(=O)OC(C)(C)C)nc2c(Br)n1 ; CC1(C)OB(/C=C2/CCOC2)OC1(C)C ; Cc1cn2cc(NC(=O)OC(C)(C)C)nc2c(/C=C2/CCOC2)n1]

To a stirred mixture of tert-butyl N-(8-bromo-6-methyl-imidazo[1,2-a]pyrazin-2-yl)carbamate (200 mg, 611.29 μmol) and 4,4,5,5-tetramethyl-2-[(Z)-tetrahydrofuran-3-ylidenemethyl]-1,3,2-dioxaborolane (154.1 mg, 733.55 μmol) in dioxane (4 mL) and H₂O (0.4 mL) were added Pd(dppf)Cl₂ (44.7 mg, 61.13 μmol) and K₂CO₃ (168.9 mg, 1.22 mmol) at room temperature under nitrogen atmosphere. The resulting mixture was stirred for 2 h at 100° C. under nitrogen atmosphere. The resulting mixture was concentrated under reduced pressure. The residue was purified by silica gel column chromatography, eluted with PE/EA (1:3) to afford tert-butyl N-[6-methyl-8-[(Z)-tetrahydrofuran-3-ylidenemethyl]imidazo[1,2-a]pyrazin-2-yl]carbamate (150 mg, 454.02 μmol, 74% yield) as a solid. LCMS (ES, m/z): 331 [M+H]⁺.

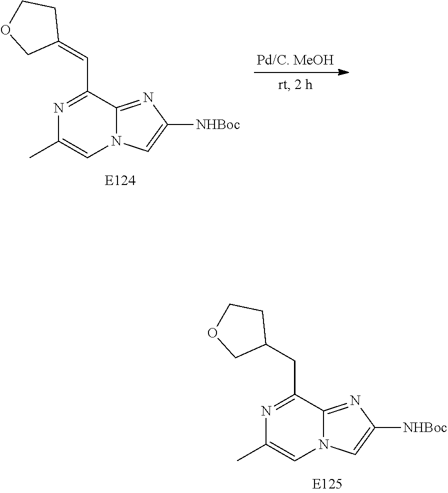 [SMILES: Cc1cn2cc(NC(=O)OC(C)(C)C)nc2c(/C=C2/CCOC2)n1 ; Cc1cn2cc(NC(=O)OC(C)(C)C)nc2c(CC2CCOC2)n1]

To a stirred mixture of tert-butyl N-[6-methyl-8-[(Z)-tetrahydrofuran-3-ylidenemethyl]imidazo[1,2-a]pyrazin-2-yl]carbamate (150 mg, 454.02 μmol) in MeOH (3 mL) was treated with Pd/C (50 mg) at rt. The resulting mixture was stirred for 2 hr at room temperature under hydrogen atmosphere. The resulting mixture was filter out Pd/C and concentrated under reduced pressure afford tert-butyl N-[6-methyl-8-(tetrahydrofuran-3-ylmethyl)imidazo[1,2-a]pyrazin-2-yl]carbamate (130 mg, 391.10 μmol, 86% yield) as a solid. LCMS (ES, m/z): 333 [M+H]⁺.

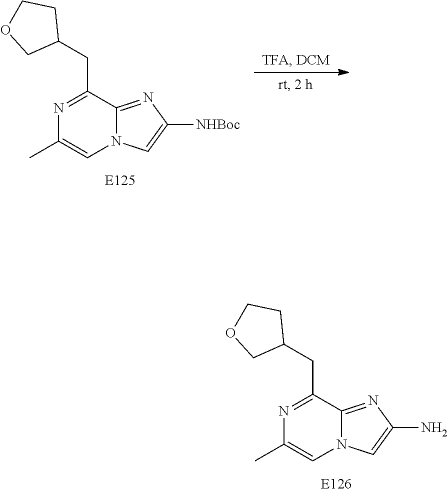 [SMILES: Cc1cn2cc(NC(=O)OC(C)(C)C)nc2c(CC2CCOC2)n1 ; Cc1cn2cc(N)nc2c(CC2CCOC2)n1]

To a stirred solution of tert-butyl N-[6-methyl-8-(tetrahydrofuran-3-ylmethyl)imidazo[1,2-a]pyrazin-2-yl]carbamate (130 mg, 391.10 μmol) in DCM (2 mL) was added TFA (1 mL) at rt. The resulting mixture was stirred for 2 h. The resulting mixture was concentrated under reduced pressure to afford 6-methyl-8-(tetrahydrofuran-3-ylmethyl)imidazo[1,2-a]pyrazin-2-amine (80 mg, 344.41 μmol, 88% yield) as a solid. LCMS (ES, m/z): 233 [M+H]⁺.

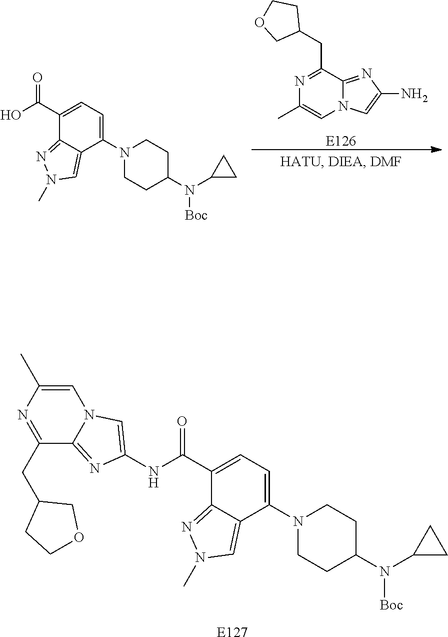

To a stirred mixture of 4-[4-[tert-butoxycarbonyl(cyclopropyl)amino]-1-piperidyl]-2-methyl-indazole-7-carboxylic acid (70 mg, 168.88 μmol) and 6-methyl-8-(tetrahydrofuran-3-ylmethyl)imidazo[1,2-a]pyrazin-2-amine (47.0 mg, 202.66 μmol) in DMF (1.5 mL) were added DIEA (65.4 mg, 506.64 μmol) and HATU (96.3 mg, 253.32 μmol) at room temperature. The resulting mixture was stirred for 3 h at rt. The resulting mixture was extracted with EA (3×10 mL). The combined organic layers were washed with water (2×10 mL), dried over anhydrous Na₂SO₄. After filtration, the filtrate was concentrated under reduced pressure. The residue was purified by silica gel column chromatography, eluted with PE/EA (1:2) to afford tert-butyl N-cyclopropyl-N-[1-[2-methyl-7-[[6-methyl-8-(tetrahydrofuran-3-ylmethyl)imidazo[1,2-a]pyrazin-2-yl]carbamoyl]indazol-4-yl]-4-piperidyl]carbamate (50 mg, 79.52 μmol, 47% yield) as a solid. LCMS (ES, m/z): 629 [M+H]⁺.

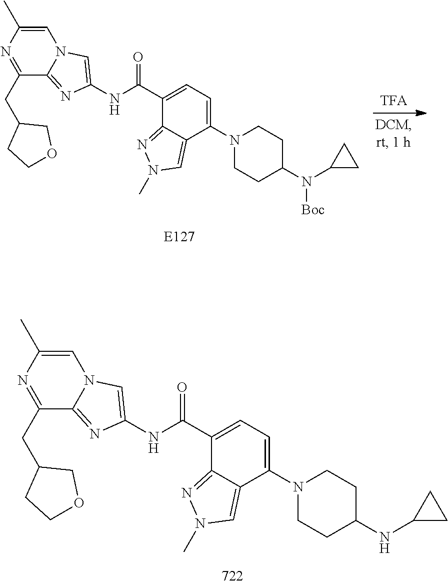 [SMILES: Cc1cn2cc(NC(=O)c3ccc(N4CCC(N(C(=O)OC(C)(C)C)C5CC5)CC4)c4cn(C)nc34)nc2c(CC2CCOC2)n1 ; "E127" ; Cc1cn2cc(NC(=O)c3ccc(N4CCC(NC5CC5)CC4)c4cn(C)nc34)nc2c(CC2CCOC2)n1 ; "722"]

To a stirred solution of tert-butyl N-cyclopropyl-N-[1-[2-methyl-7-[[6-methyl-8-(tetrahydrofuran-3-ylmethyl)imidazo[1,2-a]pyrazin-2-yl]carbamoyl]indazol-4-yl]-4-piperidyl]carbamate (50 mg, 79.52 μmol) in DCM (2 mL) was added TFA (1 mL) at rt. The resulting mixture was stirred for 2 h. The resulting mixture was concentrated under reduced pressure. The crude product was purified by Prep-HPLC with the following conditions (SunFire Prep C18 OBD Column 19*150 mm, 5 Um 10 nm, mobile phase, MeCN in water (0.05% TFA), 20% to 30% gradient in 7 min; detector, UV 254 nm) to afford 4-[4-(cyclopropylamino)-1-piperidyl]-2-methyl-N-[6-methyl-8-(tetrahydrofuran-3-ylmethyl)imidazo[1,2-a]pyrazin-2-yl]indazole-7-carboxamide 2,2,2-trifluoroacetate (7.2 mg, 13.62 μmol, 17.13% yield) as a solid. LCMS (ES, m/z): 529 [M+H]⁺. ¹H NMR (400 MHz, DMSO-d₆) δ 11.43 (s, 1H), 8.87 (s, 1H), 8.77 (s, 2H), 8.37 (d, J=4.6 Hz, 2H), 8.07 (d, J=8.1 Hz, 1H), 6.58 (d, J=8.1 Hz, 1H), 4.33 (s, 3H), 4.07 (d, J=12.9 Hz, 2H), 3.87-3.77 (m, 2H), 3.67 (q, J=7.7 Hz, 1H), 3.52-3.41 (m, 2H), 3.26-3.10 (m, 2H), 3.07 (t, J=12.3 Hz, 2H), 2.89 (p, J=7.1 Hz, 1H), 2.85-2.75 (m, 1H), 2.41 (s, 3H), 2.21 (d, J=11.7 Hz, 2H), 2.09-1.96 (m, 1H), 1.82-1.72 (m, 2H), 1.75-1.62 (m, 1H), 0.85 (d, J=7.3 Hz, 4H).

### Example 253: Synthesis of Compound 724

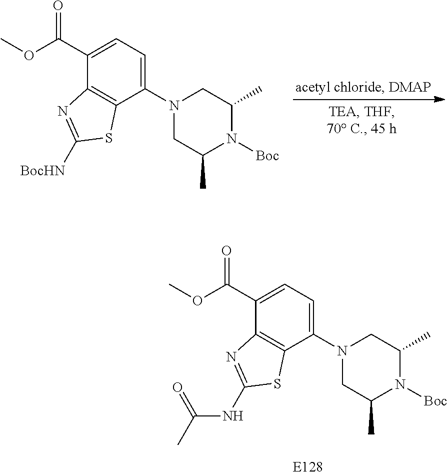 [SMILES: COC(=O)c1ccc(N2C[C@H](C)N(C(=O)OC(C)(C)C)[C@@H](C)C2)c2sc(NC(=O)OC(C)(C)C)nc12 ; COC(=O)c1ccc(N2C[C@H](C)N(C(=O)OC(C)(C)C)[C@@H](C)C2)c2sc(NC(C)=O)nc12 ; "E128"]

To a solution of methyl 2-(tert-butoxycarbonylamino)-7-[(3S,5S)-4-tert-butoxycarbonyl-3,5-dimethyl-piperazin-1-yl]-1,3-benzothiazole-4-carboxylate (340 mg, 653.04 μmol) in THF (4 mL) were added TEA (528.6 mg, 5.22 mmol), acetyl chloride (358.8 mg, 4.57 mmol) and DMAP (159.5 mg, 1.31 mmol). The mixture was stirred for 45 h at 70° C. The reaction was diluted with H₂O (15 mL). The resulting mixture was extracted with EA (2×15 mL). The combined organic layers were washed with H₂O (15 mL), dried over anhydrous Na₂SO₄. After filtration, the filtrate was concentrated under reduced pressure. The residue was purified by silica gel column chromatography, eluted with PE/EA (1/2) to afford methyl 2-acetamido-7-[(3S,5S)-4-tert-butoxycarbonyl-3,5-dimethyl-piperazin-1-yl]-1,3-benzothiazole-4-carboxylate (75 mg, 162.14 μmol, 24.83% yield) as an oil. LCMS (ES, m/z): 463 [M+H]⁺.

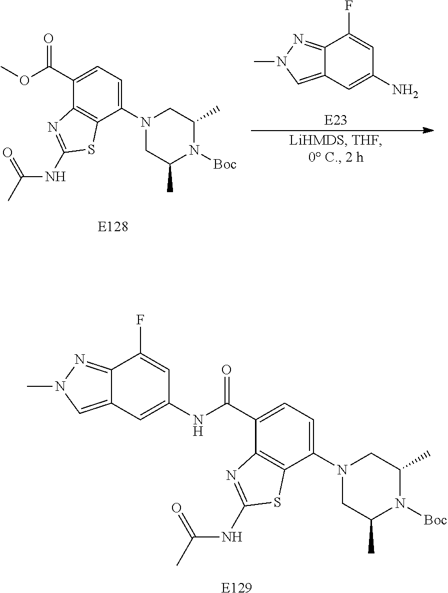

To a solution of methyl 2-acetamido-7-[(3S,5S)-4-tert-butoxycarbonyl-3,5-dimethyl-piperazin-1-yl]-1,3-benzothiazole-4-carboxylate (75 mg, 162.14 μmol) in THF (0.8 mL) was added 7-fluoro-2-methyl-indazol-5-amine (34.8 mg, 210.78 μmol) at rt. Then LiHMDS (1 M, 650 μL) was added dropwise at 0° C. under N₂. The mixture was stirred for 2 h at rt. The reaction was quenched with NH₄Cl (aq.). The resulting mixture was extracted with DCM (2×15 mL). The combined organic layers were washed with H₂O (10 mL), dried over anhydrous Na₂SO₄. After filtration, the filtrate was concentrated under reduced pressure. The residue was purified by silica gel column chromatography, eluted with DCM/EA (1/3) to afford tert-butyl (2S,6S)-4-[2-acetamido-4-[(7-fluoro-2-methyl-indazol-5-yl)carbamoyl]-1,3-benzothiazol-7-yl]-2,6-dimethyl-piperazine-1-carboxylate (95 mg, 159.48 μmol, 98% yield) as a solid. LCMS (ES, m/z): 596 [M+H]⁺.

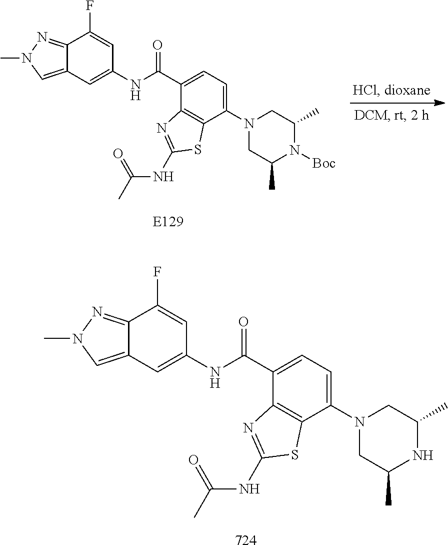

To a solution of tert-butyl (2S,6S)-4-[2-acetamido-4-[(7-fluoro-2-methyl-indazol-5-yl)carbamoyl]-1,3-benzothiazol-7-yl]-2,6-dimethyl-piperazine-1-carboxylate (95 mg, 159.48 μmol) in DCM (1 mL) was added HCl (4.0 M in 1,4-dioxane) (400 μL). The mixture was stirred for 2 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃·H₂O), 55% to 75% gradient in 7 min; detector, UV 254 nm) to afford 2-acetamido-7-[(3S,5S)-3,5-dimethylpiperazin-1-yl]-N-(7-fluoro-2-methyl-indazol-5-yl)-1,3-benzothiazole-4-carboxamide (40 mg, 80.71 μmol, 51% yield) as a solid. LCMS (ES, m/z): 496 [M+H]⁺. ¹H NMR (400 MHz, DMSO-d₆) δ 11.86 (s, 1H), 8.43 (d, J=2.8 Hz, 1H), 8.23 (d, J=1.7 Hz, 1H), 8.13 (d, J=8.3 Hz, 1H), 7.53 (dd, J=13.1, 1.7 Hz, 1H), 7.00 (d, J=8.5 Hz, 1H), 4.18 (s, 3H), 3.31-3.21 (m, 2H), 3.15 (dd, J=11.6, 3.1 Hz, 2H), 2.96 (dd, J=11.5, 6.1 Hz, 2H), 2.32 (s, 3H), 1.18 (d, J=6.4 Hz, 6H).

### Example 254: Synthesis of Compound 725

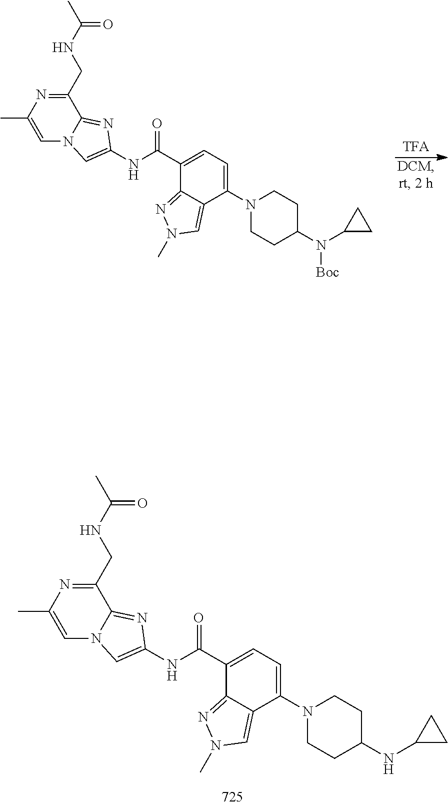 [SMILES: CC(=O)NCc1nc(C)cn2cc(NC(=O)c3ccc(N4CCC(N(C(=O)OC(C)(C)C)C5CC5)CC4)c4cn(C)nc34)nc12 ; "DCM,rt, 2 h" ; CC(=O)NCc1nc(C)cn2cc(NC(=O)c3ccc(N4CCC(NC5CC5)CC4)c4cn(C)nc34)nc12 ; "TFA"]

To a stirred mixture of tert-butyl N-[1-[7-[[8-(acetamidomethyl)-6-methyl-imidazo[1,2-a]pyrazin-2-yl]carbamoyl]-2-methyl-indazol-4-yl]-4-piperidyl]-N-cyclopropyl-carbamate (20 mg, 32.48 μmol) in DCM (0.5 mL) was added TFA 0.25 mL) dropwise at room temperature. The resulting mixture was stirred for 2 h at room temperature. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 30% to 80% gradient in 7 min; detector, UV 254 nm) to afford N-[8-(acetamidomethyl)-6-methyl-imidazo[1,2-a]pyrazin-2-yl]-4-[4-(cyclopropylamino)-1-piperidyl]-2-methyl-indazole-7-carboxamide (3.1 mg, 6.01 μmol, 19% yield) as a solid. LCMS (ES, m/z): 516 [M+H]⁺. ¹H NMR (400 MHz, DMSO-d₆) δ 11.49 (s, 1H), 8.80 (s, 1H), 8.38 (t, J=7.6 Hz, 3H), 8.03 (d, J=8.2 Hz, 1H), 6.51 (d, J=8.2 Hz, 1H), 4.72 (d, J=5.5 Hz, 2H), 4.31 (s, 3H), 3.90 (d, J=12.8 Hz, 2H), 3.08 (t, J=11.8 Hz, 2H), 2.79 (s, 1H), 2.42 (s, 3H), 2.13 (dt, J=6.6, 3.1 Hz, 1H), 2.05-1.97 (m, 2H), 1.94 (s, 3H), 1.48 (q, J=10.4, 9.6 Hz, 2H), 0.45-0.36 (m, 2H), 0.24 (p, J=4.0 Hz, 2H).

### Example 255: Synthesis of Compound 726

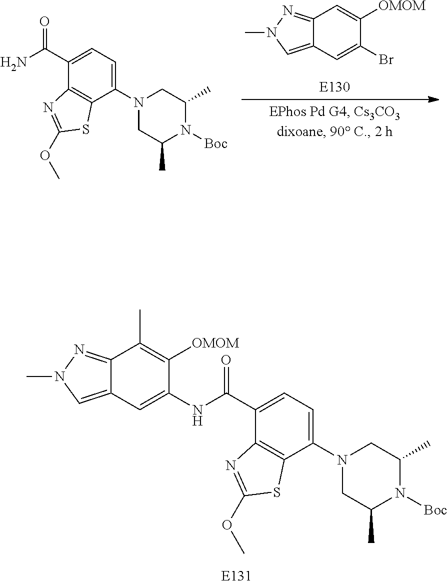

To a stirred mixture of tert-butyl (2S,6S)-4-(4-carbamoyl-2-methoxy-1,3-benzothiazol-7-yl)-2,6-dimethyl-piperazine-1-carboxylate (60 mg, 142.68 μmol) and 5-bromo-6-(methoxymethoxy)-2,7-dimethyl-indazole (48.8 mg, 171.21 μmol) in Dioxane (1.2 mL) were added Cs₂CO₃ (139.5 mg, 428.04 μmol) and EPhos Pd G4 (13.1 mg, 14.27 μmol) at room temperature under nitrogen atmosphere. The resulting mixture was concentrated under reduced pressure. The residue was purified by silica gel column chromatography, eluted with DCM/MeOH (10:1) to afford tert-butyl (2S,6S)-4-[2-methoxy-4-[[6-(methoxymethoxy)-2,7-dimethyl-indazol-5-yl]carbamoyl]-1,3-benzothiazol-7-yl]-2,6-dimethyl-piperazine-1-carboxylate (73 mg, 116.85 μmol, 81.89% yield) as a solid. LCMS (ES, m/z): 625 [M+H]⁺.

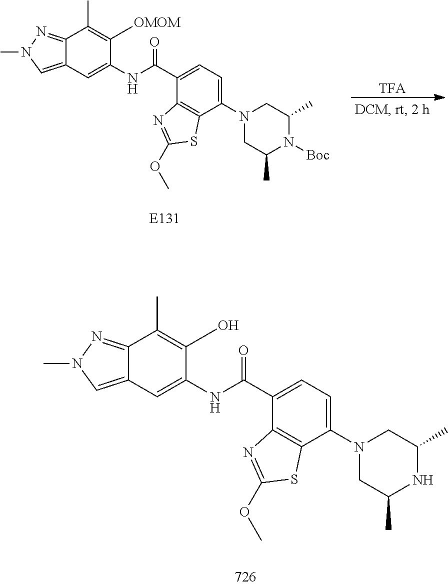

To a stirred mixture of tert-butyl (2S,6S)-4-[2-methoxy-4-[[6-(methoxymethoxy)-2,7-dimethyl-indazol-5-yl]carbamoyl]-1,3-benzothiazol-7-yl]-2,6-dimethyl-piperazine-1-carboxylate (73 mg, 116.85 μmol) in DCM (1.50 mL) was added TFA (0.5 mL) dropwise at room temperature. The resulting mixture was stirred for 2 h at room temperature. The resulting mixture were concentrated under reduced pressure. The crude product was purified by Prep-HPLC with the following conditions (column, C18 silica gel, XBridge, 19×150 mm; mobile phase, MeCN in water (0.5% NH₃·H₂O), 40% to 90% gradient in 7 min; detector, UV 254 nm) to afford 7-[(3S,5S)-3,5-dimethylpiperazin-1-yl]-N-(6-hydroxy-2,7-dimethyl-indazol-5-yl)-2-methoxy-1,3-benzothiazole-4-carboxamide (14.3 mg, 29.76 μmol, 25% yield) as a solid. LCMS (ES, m/z): 481 [M+H]⁺. ¹H NMR (400 MHz, DMSO-d₆) δ 11.75 (s, 1H), 9.08 (s, 1H), 8.72 (s, 1H), 8.21 (d, J=8.5 Hz, 1H), 8.15 (s, 1H), 7.06 (d, J=8.5 Hz, 1H), 4.43 (s, 3H), 4.09 (s, 3H), 3.25 (td, J=6.2, 3.1 Hz, 2H), 3.11 (dd, J=11.7, 3.1 Hz, 2H), 2.92 (dd, J=11.4, 6.1 Hz, 2H), 2.42 (s, 3H), 1.18 (d, J=6.4 Hz, 6H).

### Example 256: Synthesis of Compound 727

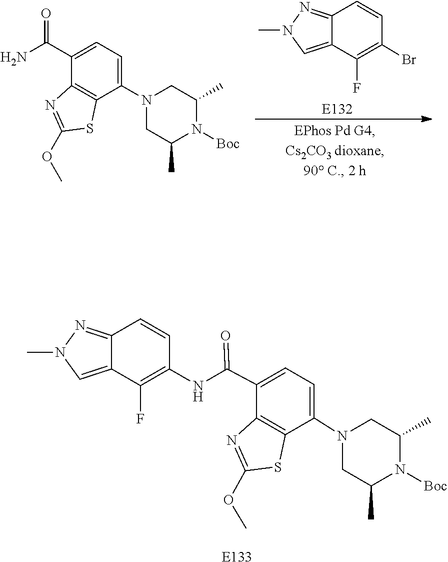

To a stirred mixture of tert-butyl (2S,6S)-4-(4-carbamoyl-2-methoxy-1,3-benzothiazol-7-yl)-2,6-dimethyl-piperazine-1-carboxylate (40 mg, 95.12 μmol) and 5-bromo-4-fluoro-2-methyl-indazole (26.1 mg, 114.14 μmol) in Dioxane were added Cs₂CO₃ (93.0 mg, 285.36 μmol) and EPhos Pd G4 (8.7 mg, 9.51 μmol) at room temperature under nitrogen atmosphere. The mixture was stirred for 2 h at 90° C. The resulting mixture was concentrated under reduced pressure. The residue was purified by silica gel column chromatography, eluted with DCM/MeOH (10:1) to afford tert-butyl (2S,6S)-4-[4-[(4-fluoro-2-methyl-indazol-5-yl)carbamoyl]-2-methoxy-1,3-benzothiazol-7-yl]-2,6-dimethyl-piperazine-1-carboxylate (38 mg, 66.82 μmol, 70% yield) as a solid. LCMS (ES, m/z): 569 [M+H]⁺.

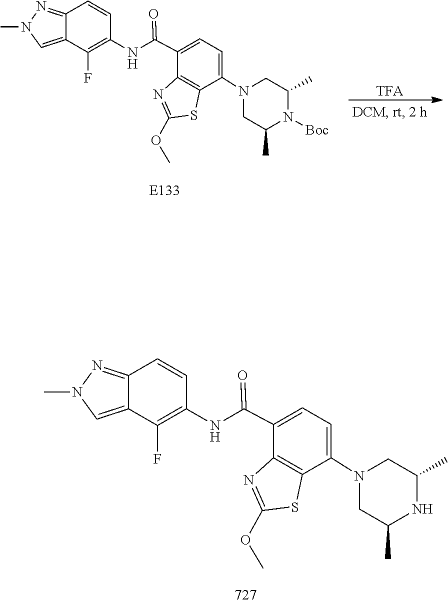

To a stirred mixture of tert-butyl (2S,6S)-4-[4-[(4-fluoro-2-methyl-indazol-5-yl)carbamoyl]-2-methoxy-1,3-benzothiazol-7-yl]-2,6-dimethyl-piperazine-1-carboxylate (38 mg, 66.82 μmol) in DCM (1 mL) was added TFA (0.25 mL) dropwise at room temperature. The resulting mixture was stirred for 2 h at room temperature. The resulting mixture were concentrated under reduced pressure. The crude product was purified by Prep-HPLC with the following conditions (column, C18 silica gel, XBridge, 19×150 mm; mobile phase, MeCN in water (0.05% TFA), 30% to 70% gradient in 7 min; detector, UV 254 nm) to afford 7-((3S,5S)-3,5-dimethylpiperazin-1-yl)-N-(4-fluoro-2-methyl-2H-indazol-5-yl)-2-methoxybenzo[d]thiazole-4-carboxamide 2,2,2-trifluoroacetate (12 mg, 20.60 μmol, 31% yield) as a solid. LCMS (ES, m/z): 469 [M+H]⁺. ¹H NMR (400 MHz, DMSO-d₆) δ 11.54 (s, 1H), 9.00 (s, 2H), 8.54 (s, 1H), 8.29 (dd, J=9.2, 7.4 Hz, 1H), 8.22 (d, J=8.5 Hz, 1H), 7.50 (d, J=9.3 Hz, 1H), 7.20 (d, J=8.5 Hz, 1H), 4.39 (s, 3H), 4.19 (s, 3H), 3.78 (s, 2H), 3.41-3.33 (m, 2H), 3.25 (dd, J=13.1, 6.3 Hz, 2H), 1.42 (d, J=6.5 Hz, 6H).

### Example 257: Synthesis of Compound 728

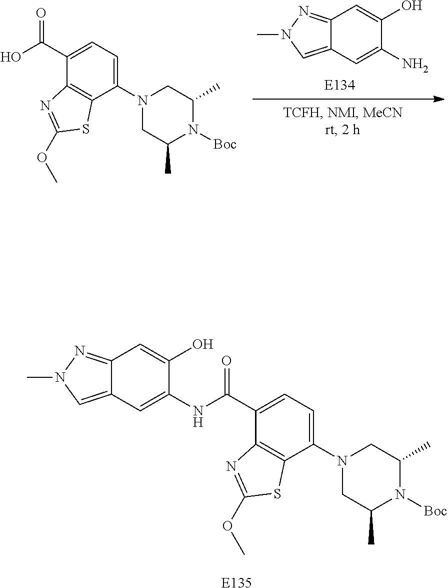

To a stirred mixture of 7-[(3S,5S)-4-tert-butoxycarbonyl-3,5-dimethyl-piperazin-1-yl]-2-methoxy-1,3-benzothiazole-4-carboxylic acid (50 mg, 118.62 μmol) and 5-amino-2-methyl-indazol-6-ol (23.2 mg, 142.35 μmol) in MeCN (1 mL) were added NMI (29.2 mg, 355.86 μmol) and TCFH (49.9 mg, 177.93 μmol) at room temperature. The resulting mixture was stirred for 3 h at rt. The resulting mixture was extracted with EA (1×10 mL). The combined organic layers were washed with water (2×10 mL), dried over anhydrous Na₂SO₄. After filtration, the filtrate was concentrated under reduced pressure. The residue was purified by silica gel column chromatography, eluted with PE/EA (1:2) to afford tert-butyl (2S,6S)-4-[4-[(6-hydroxy-2-methyl-indazol-5-yl)carbamoyl]-2-methoxy-1,3-benzothiazol-7-yl]-2,6-dimethyl-piperazine-1-carboxylate (40 mg, 70.59 μmol, 60% yield) as a solid. LCMS (ES, m/z): 567 [M+H]⁺.

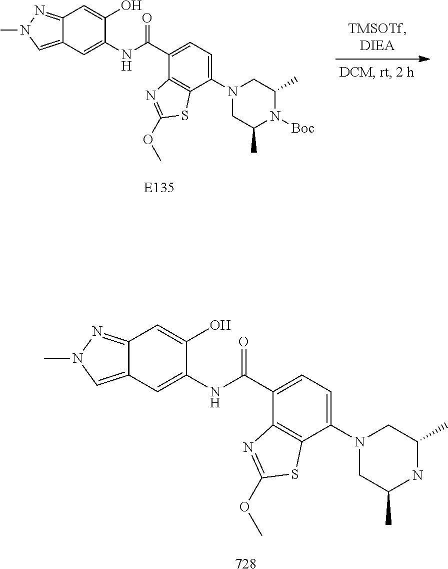

To a stirred mixture of tert-butyl (2S,6S)-4-[4-[(6-hydroxy-2-methyl-indazol-5-yl)carbamoyl]-2-methoxy-1,3-benzothiazol-7-yl]-2,6-dimethyl-piperazine-1-carboxylate (40 mg, 70.59 μmol) in DCM (1 mL) was added DIEA (18.2 mg, 141.18 μmol) and TMSOTf (47.0 mg, 211.76 μmol) dropwise at room temperature. The resulting mixture was stirred for 2 h at room temperature. The resulting mixture were concentrated under reduced pressure and diluted with water (2 mL). The mixture was basified to pH 8 with saturated NaHCO₃ (aq.) and extracted with DCM (3×3 mL). The combined organic layers were washed with water (2×10 mL) and brine (1×10 mL), dried over anhydrous Na₂SO₄. After filtration, the filtrate was concentrated under reduced pressure. The crude product was purified by Prep-HPLC with the following conditions (column, C18 silica gel, XBridge, 19×150 mm; mobile phase, MeCN in water (0.05% NH₃·H₂O), 40% to 50% gradient in 7 min; detector, UV 254 nm) to afford 7-[(3S,5S)-3,5-dimethylpiperazin-1-yl]-N-(6-hydroxy-2-methyl-indazol-5-yl)-2-methoxy-1,3-benzothiazole-4-carboxamide (24.9 mg, 53.37 μmol, 76% yield) as a solid. LCMS (ES, m/z): 467 [M+H]⁺. ¹H NMR (400 MHz, DMSO-d₆) δ 11.76 (s, 1H), 10.47 (s, 1H), 8.83 (s, 1H), 8.21 (d, J=8.5 Hz, 1H), 8.15 (s, 1H), 7.06 (d, J=8.6 Hz, 1H), 6.92 (s, 1H), 4.41 (s, 3H), 4.06 (s, 3H), 3.29-3.22 (m, 2H), 3.11 (dd, J=11.7, 3.2 Hz, 2H), 2.93 (dd, J=11.4, 6.1 Hz, 2H), 1.18 (d, J=6.4 Hz, 6H).

### Example 258: Synthesis of Compound 729

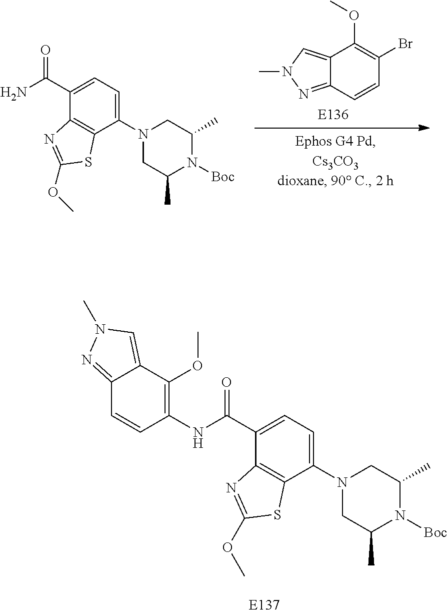

To a stirred mixture of tert-butyl (2S,6S)-4-(4-carbamoyl-2-methoxy-1,3-benzothiazol-7-yl)-2,6-dimethyl-piperazine-1-carboxylate (50 mg, 118.90 μmol) and 5-bromo-4-methoxy-2-methyl-indazole (34.4 mg, 142.68 μmol) in Dioxane (1 mL) were added Cs₂CO₃ (77.4 mg, 237.80 μmol) and Ephos G4 Pd (10.9 mg, 11.89 μmol) at room temperature under nitrogen atmosphere. The resulting mixture was stirred for 2 h at 100° C. under nitrogen atmosphere. The resulting mixture was concentrated under reduced pressure. The residue was purified by silica gel column chromatography, eluted with PE/EA (1:3) to afford tert-butyl (2S,6S)-4-[2-methoxy-4-[(4-methoxy-2-methyl-indazol-5-yl)carbamoyl]-1,3-benzothiazol-7-yl]-2,6-dimethyl-piperazine-1-carboxylate (40 mg, 68.88 μmol, 58% yield) as a light solid. LCMS (ES, m/z): 581 [M+H]⁺.

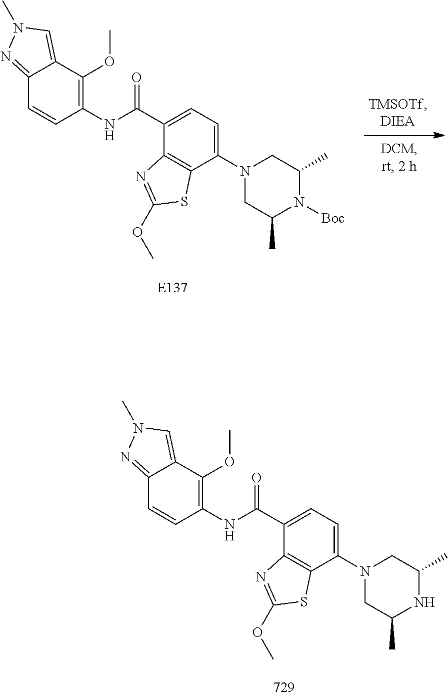

To a stirred mixture of tert-butyl (2S,6S)-4-[2-methoxy-4-[(4-methoxy-2-methyl-indazol-5-yl)carbamoyl]-1,3-benzothiazol-7-yl]-2,6-dimethyl-piperazine-1-carboxylate (40 mg, 68.88 μmol) in DCM (1 mL) was added DIEA (17.8 mg, 137.77 μmol) and TMSOTf (45.9 mg, 206.65 μmol) dropwise at room temperature. The resulting mixture was stirred for 2 h at room temperature. The resulting mixture were concentrated under reduced pressure. The residue was purified by Prep-HPLC with the following conditions (column, C18 silica gel, XBridge, 19×150 mm; mobile phase, MeCN in water (0.05% NH₃·H₂O), 40% to 50% gradient in 7 min; detector, UV 254 nm) to afford 7-[(3S,5S)-3,5-dimethylpiperazin-1-yl]-2-methoxy-N-(4-methoxy-2-methyl-indazol-5-yl)-1,3-benzothiazole-4-carboxamide (13.9 mg, 28.92 μmol, 42% yield) as a solid. LCMS (ES, m/z): 481 [M+H]⁺. ¹H NMR (400 MHz, DMSO-d₆) δ 11.43 (s, 1H), 8.64 (s, 1H), 8.31 (d, J=9.2 Hz, 1H), 8.20 (d, J=8.5 Hz, 1H), 7.30 (d, J=9.3 Hz, 1H), 7.06 (d, J=8.6 Hz, 1H), 4.40 (s, 3H), 4.15 (d, J=15.9 Hz, 6H), 3.24 (tt, J=9.2, 4.5 Hz, 2H), 3.11 (dd, J=11.5, 3.1 Hz, 2H), 2.92 (dd, J=11.4, 6.1 Hz, 2H), 1.18 (d, J=6.4 Hz, 6H).

### Example 259: Synthesis of Compound 730

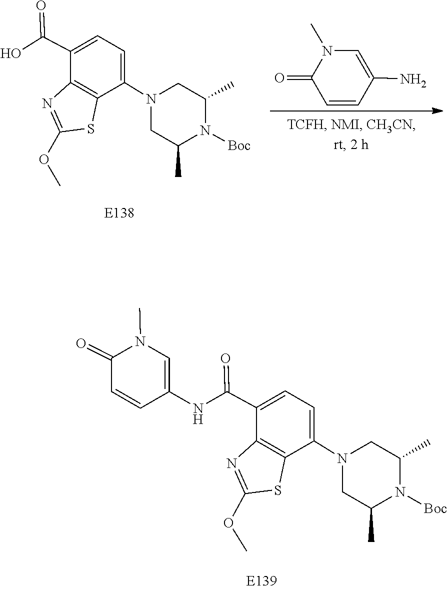

To a stirred mixture of 7-[(3S,5S)-4-tert-butoxycarbonyl-3,5-dimethyl-piperazin-1-yl]-2-methoxy-1,3-benzothiazole-4-carboxylic acid (0.05 g, 118.62 μmol) and 5-amino-1-methyl-pyridin-2-one (17.7 mg, 142.35 μmol) in MeCN (2 mL) were added NMI (29.2 mg, 355.86 μmol), TCFH (49.9 mg, 177.93 μmol) at room temperature. The resulting mixture was stirred for 2 h at room temperature. The resulting mixture was diluted with water (3 mL). The resulting mixture was extracted with EA (3×3 mL). The combined organic layers were washed with water (1×8 mL), brine (1×8 mL), dried over anhydrous Na₂SO₄. After filtration, the filtrate was concentrated under reduced pressure. The residue was purified by silica gel column chromatography, eluted with PE/EA (1:10) to afford tert-butyl (2S,6S)-4-[2-methoxy-4-[(1-methyl-6-oxo-3-pyridyl)carbamoyl]-1,3-benzothiazol-7-yl]-2,6-dimethyl-piperazine-1-carboxylate (0.051 g, 96.66 μmol, 81% yield) as a solid. LCMS (ES, m/z): 528 [M+H]⁺.

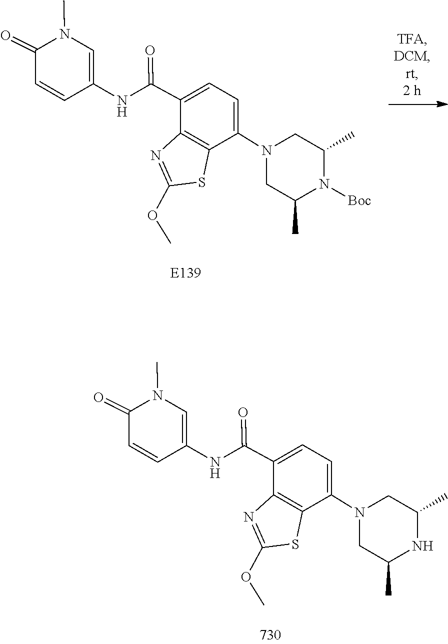 [SMILES: COc1nc2c(C(=O)Nc3ccc(=O)n(C)c3)ccc(N3C[C@H](C)N(C(=O)OC(C)(C)C)[C@@H](C)C3)c2s1 ; COc1nc2c(C(=O)Nc3ccc(=O)n(C)c3)ccc(N3C[C@H](C)N[C@@H](C)C3)c2s1]

To a solution of tert-butyl (2S,6S)-4-[2-methoxy-4-[(1-methyl-6-oxo-3-pyridyl)carbamoyl]-1,3-benzothiazol-7-yl]-2,6-dimethyl-piperazine-1-carboxylate (0.051 g, 96.66 μmol) in DCM (1 mL) was added TFA (0.4 mL). The reaction was stirred for 2 h at rt. The resulting mixture was concentrated under reduced pressure and adjusted to pH 8 with saturated NaHCO₃ (aq.). After filtration, the filtrate was concentrated under reduced pressure. The residue was purified by reversed-phase flash chromatography with the following conditions (SunFire Prep C18 OBD Column 19*150 mm, 5 μm 10 nm, mobile phase, MeCN in water (0.05% TFA), 10% to 30% gradient in 7 min; detector, UV 254 nm) to afford 7-((3S,5S)-3,5-dimethylpiperazin-1-yl)-2-methoxy-N-(1-methyl-6-oxo-1,6-dihydropyridin-3-yl)benzo[d]thiazole-4-carboxamide 2,2,2-trifluoroacetate (0.014 g, 25.85 μmol, 27% yield) as a solid. LCMS (ES, m/z): 428 [M+H]⁺. ¹H NMR (400 MHz, Methanol-d₄) δ 8.46 (d, J=2.9 Hz, 1H), 8.21 (dd, J=8.7, 3.8 Hz, 1H), 7.62-7.52 (m, 1H), 7.14 (dd, J=8.6, 3.7 Hz, 1H), 6.56 (s, 1H), 4.38 (d, J=1.6 Hz, 3H), 3.86 (tt, J=10.1, 5.0 Hz, 2H), 3.60 (d, J=3.5 Hz, 3H), 3.43 (dd, J=13.2, 3.2 Hz, 2H), 3.34-3.24 (m, 2H), 1.54 (d, J=6.6 Hz, 6H).

### Example 260: Synthesis of Compound 731

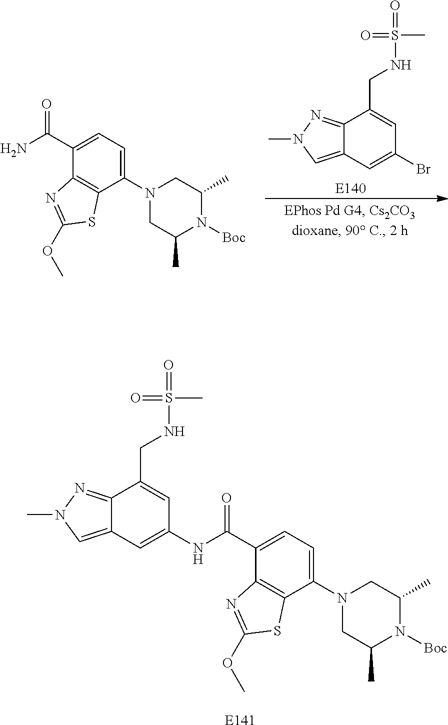

To a stirred mixture of tert-butyl (2S,6S)-4-(4-carbamoyl-2-methoxy-1,3-benzothiazol-7-yl)-2,6-dimethyl-piperazine-1-carboxylate (0.045 g, 107.01 μmol) and N-[(5-bromo-2-methyl-indazol-7-yl)methyl]methanesulfonamide (40.9 mg, 128.41 μmol) in Dioxane (1 mL) were added Cs₂CO₃ (69.7 mg, 214.02 μmol), EPhos Pd G4 (9.8 mg, 10.70 μmol) at room temperature under nitrogen atmosphere. The resulting mixture was stirred for 2 h at 90° C. under nitrogen atmosphere. The mixture was allowed to cool down to room temperature. After filtration, the filtrate was concentrated under reduced pressure. The residue was purified by silica gel column chromatography, eluted with PE/EA (1:8) to afford tert-butyl (2S,6S)-4-[4-[[7-(methanesulfonamidomethyl)-2-methyl-indazol-5-yl]carbamoyl]-2-methoxy-1,3-benzothiazol-7-yl]-2,6-dimethyl-piperazine-1-carboxylate (0.053 g, 80.57 μmol, 75% yield) as a solid. LCMS (ES, m/z): 658 [M+H]⁺.

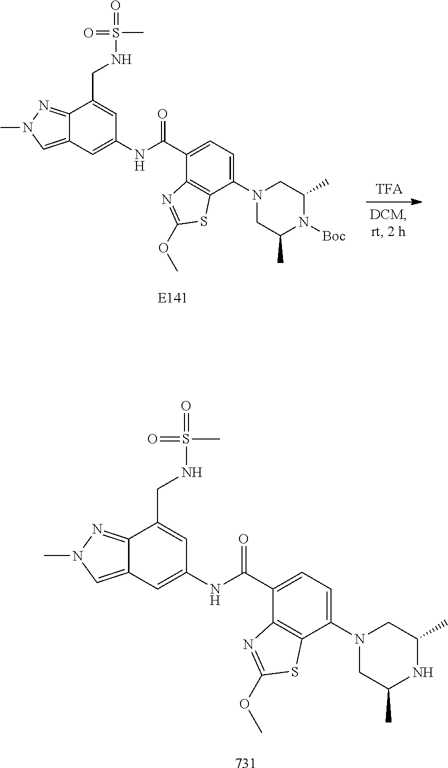

To a solution of tert-butyl (2S,6S)-4-[4-[[7-(methanesulfonamidomethyl)-2-methyl-indazol-5-yl]carbamoyl]-2-methoxy-1,3-benzothiazol-7-yl]-2,6-dimethyl-piperazine-1-carboxylate (0.053 g, 80.57 μmol) in DCM (1 mL) was added TFA (0.4 mL). The reaction was stirred for 2 h at rt. The resulting mixture was concentrated under reduced pressure and adjusted to pH 8 with saturated NaHCO₃ (aq.). After filtration, the filtrate was concentrated under reduced pressure. The residue was purified by reversed-phase flash chromatography with the following conditions (SunFire Prep C18 OBD Column 19*150 mm, 5 μm 10 nm, mobile phase, MeCN in water (0.05% TFA), 10% to 30% gradient in 7 min; detector, UV 254 nm) to afford 7-((3S,5S)-3,5-dimethylpiperazin-1-yl)-2-methoxy-N-(2-methyl-7-(methylsulfonamidomethyl)-2H-indazol-5-yl)benzo[d]thiazole-4-carboxamide 2,2,2-trifluoroacetate (0.03 g, 44.66 μmol, 55% yield) as a solid. LCMS (ES, m/z): 558 [M+H]⁺. ¹H NMR (400 MHz, Methanol-d₄) δ 11.84 (s, 1H), 8.48 (s, 1H), 8.24 (d, J=13.9 Hz, 2H), 7.40 (s, 1H), 7.18 (s, 1H), 4.65 (s, 2H), 4.50 (s, 3H), 4.47 (s, 3H), 3.81 (s, 2H), 3.42 (d, J=13.3 Hz, 2H), 3.16 (s, 3H), 3.03 (s, 3H), 1.53 (t, J=6.3 Hz, 6H).

### Example 261: Synthesis of Compound 733

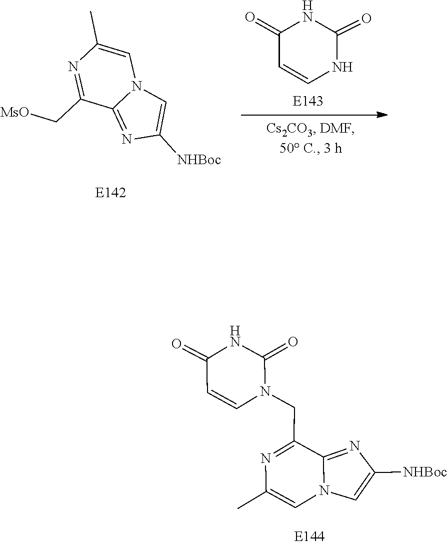 [SMILES: Cc1cn2cc(NC(=O)OC(C)(C)C)nc2c(COS(C)(=O)=O)n1 ; O=c1cc[nH]c(=O)[nH]1 ; Cc1cn2cc(NC(=O)OC(C)(C)C)nc2c(Cn2ccc(=O)[nH]c2=O)n1]

To a stirred solution of [2-(tert-butoxycarbonylamino)-6-methyl-imidazo[1,2-a]pyrazin-8-yl]methyl methanesulfonate (100 mg, 280.59 μmol) and 1H-pyrimidine-2,4-dione (40.8 mg, 364.76 μmol) in DMF (2 mL) was added Cs₂CO₃ (182.8 mg, 561.17 μmol) at rt. The resulting mixture was stirred for 3 h at 50° C. The resulting mixture was diluted with water (8 mL). The resulting mixture was extracted with EA (2×10 mL). The combined organic layers were washed with water (2×10 mL) and brine (1×10 mL), dried over anhydrous Na₂SO₄. After filtration, the filtrate was concentrated under reduced pressure. The residue was purified by silica gel column chromatography, eluted with PE/EA (2:1) to afford tert-butyl N-[8-[(2,4-dioxopyrimidin-1-yl)methyl]-6-methyl-imidazo[1,2-a]pyrazin-2-yl]carbamate (91 mg, 244.38 μmol, 87% yield) as a solid. LCMS (ES, m/z): 373 [M+H]⁺.

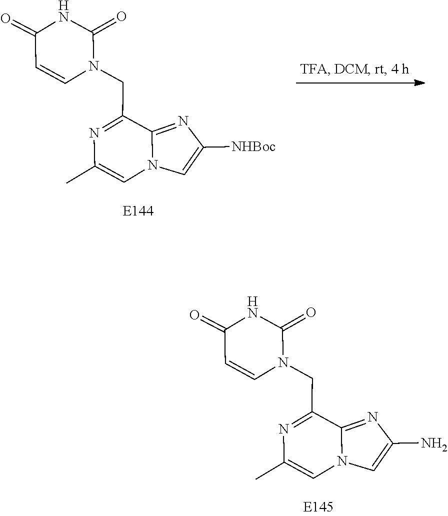 [SMILES: Cc1cn2cc(NC(=O)OC(C)(C)C)nc2c(Cn2ccc(=O)[nH]c2=O)n1 ; Cc1cn2cc(N)nc2c(Cn2ccc(=O)[nH]c2=O)n1]

A solution of tert-butyl N-[8-[(2,4-dioxopyrimidin-1-yl)methyl]-6-methyl-imidazo[1,2-a]pyrazin-2-yl]carbamate (76 mg, 204.09 μmol) in DCM (1 mL) was treated with TFA (0.5 mL) at room temperature. The resulting mixture was stirred for 4 h at room temperature under nitrogen atmosphere. The resulting mixture was concentrated under reduced pressure. The resulting mixture was diluted with water (3 mL). The residue was basified to pH 8 with saturated NaHCO₃ (aq.). The resulting mixture was extracted with CH₂Cl₂:MeOH=10:1 (2×8 mL). The combined organic layers were washed with brine (1×6 mL), dried over anhydrous Na₂SO₄. After filtration, the filtrate was concentrated under reduced pressure to afford 1-[(2-amino-6-methyl-imidazo[1,2-a]pyrazin-8-yl)methyl]pyrimidine-2,4-dione (36 mg, 132.23 μmol, 65% yield) as a solid. LCMS (ES, m/z): 273 [M+H]⁺.

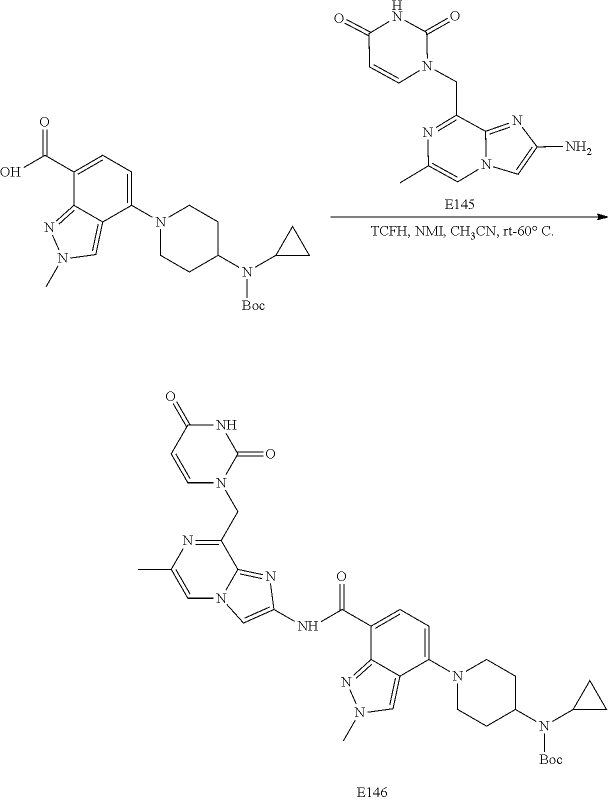 [SMILES: Cn1cc2c(N3CCC(N(C(=O)OC(C)(C)C)C4CC4)CC3)ccc(C(=O)O)c2n1 ; Cc1cn2cc(N)nc2c(Cn2ccc(=O)[nH]c2=O)n1 ; Cc1cn2cc(NC(=O)c3ccc(N4CCC(N(C(=O)OC(C)(C)C)C5CC5)CC4)c4cn(C)nc34)nc2c(Cn2ccc(=O)[nH]c2=O)n1]

To a stirred mixture of 1-[(2-amino-6-methyl-imidazo[1,2-a]pyrazin-8-yl)methyl]pyrimidine-2,4-dione (13.1 mg, 48.25 μmol) and 4-[4-[tert-butoxycarbonyl(cyclopropyl)amino]-1-piperidyl]-2-methyl-indazole-7-carboxylic acid (20 mg, 48.25 μmol) in CH₃CN (0.6 mL) were added NMI (11.8 mg, 144.75 μmol) and TCFH (20.3 mg, 72.38 μmol) at room temperature. The resulting mixture was stirred for 4 h at 60° C. The resulting mixture was diluted with water (10 mL). The resulting mixture was extracted with EA (3×15 mL). The combined organic layers were washed with brine (1×20 mL), dried over anhydrous Na₂SO₄. After filtration, the filtrate was concentrated under reduced pressure. The residue was purified by silica gel column chromatography, eluted with EA to afford tert-butyl N-cyclopropyl-N-[1-[7-[[8-[(2,4-dioxopyrimidin-1-yl)methyl]-6-methyl-imidazo[1,2-a]pyrazin-2-yl]carbamoyl]-2-methyl-indazol-4-yl]-4-piperidyl]carbamate (14 mg, 20.93 μmol, 43% yield) as a solid. LCMS (ES, m/z): 669 [M+H]⁺.

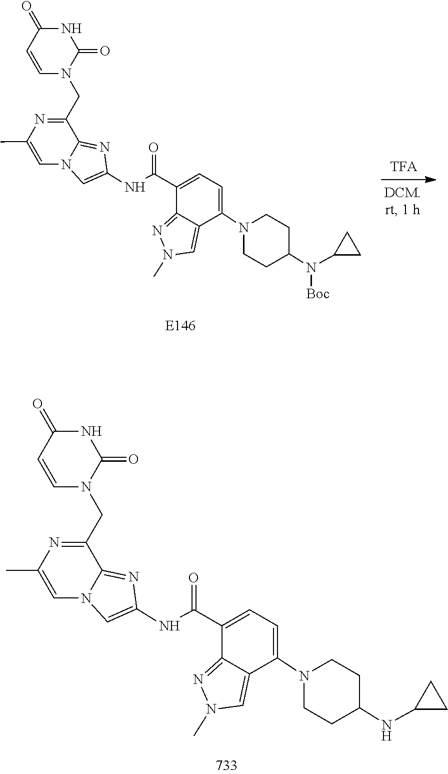 [SMILES: Cc1cn2cc(NC(=O)c3ccc(N4CCC(N(C(=O)OC(C)(C)C)C5CC5)CC4)c4cn(C)nc34)nc2c(Cn2ccc(=O)[nH]c2=O)n1 ; Cc1cn2cc(NC(=O)c3ccc(N4CCC(NC5CC5)CC4)c4cn(C)nc34)nc2c(Cn2ccc(=O)[nH]c2=O)n1]

A solution of tert-butyl N-cyclopropyl-N-[1-[7-[[8-[(2,4-dioxopyrimidin-1-yl)methyl]-6-methyl-imidazo[1,2-a]pyrazin-2-yl]carbamoyl]-2-methyl-indazol-4-yl]-4-piperidyl]carbamate (14 mg, 20.93 μmol) in DCM (0.3 mL) was treated with TFA (0.3 mL) at room temperature. The resulting mixture was stirred for 1 h at room temperature under nitrogen atmosphere. The resulting mixture was concentrated under reduced pressure. The crude product was purified by Prep-HPLC with the following conditions (SunFire Prep C18 OBD Column 19*150 mm, 5 μm 10 nm, mobile phase, MeCN in water (0.05% TFA), 20% to 40% gradient in 25 min; detector, UV 254 nm) to afford 4-(4-(cyclopropylamino)piperidin-1-yl)-N-(8-((2,4-dioxo-3,4-dihydropyrimidin-1(2H)-yl)methyl)-6-methylimidazo[1,2-a]pyrazin-2-yl)-2-methyl-2H-indazole-7-carboxamide 2,2,2-trifluoroacetate (7 mg, 10.25 μmol, 49% yield) as a solid. LCMS (ES, m/z): 569 [M+H]⁺. ¹H NMR (400 MHz, DMSO-d₆) δ 11.52 (s, 1H), 11.29 (d, J=2.3 Hz, 1H), 8.86 (s, 1H), 8.69 (s, 2H), 8.41 (s, 1H), 8.38 (s, 1H), 8.04 (d, J=8.1 Hz, 1H), 7.78 (d, J=7.9 Hz, 1H), 6.56 (d, J=8.2 Hz, 1H), 5.66 (dd, J=7.8, 2.3 Hz, 1H), 5.34 (s, 2H), 4.31 (s, 3H), 4.05 (d, J=13.0 Hz, 2H), 3.05 (t, J=12.4 Hz, 2H), 2.78 (s, 1H), 2.34 (s, 3H), 2.18 (d, J=12.0 Hz, 2H), 1.81-1.62 (m, 2H), 0.82 (t, J=7.0 Hz, 4H).

### Example 262: Synthesis of Compound 734

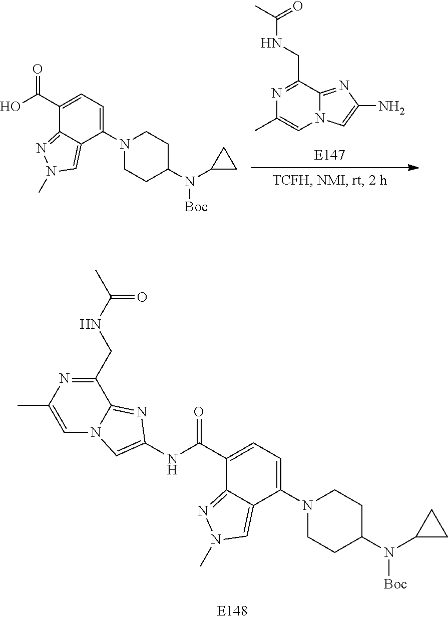

To a stirred mixture of 4-[4-[tert-butoxycarbonyl(cyclopropyl)amino]-1-piperidyl]-2-methyl-indazole-7-carboxylic acid (120 mg, 289.51 μmol) and N-[(2-amino-6-methyl-imidazo[1,2-a]pyrazin-8-yl)methyl]acetamide (76.2 mg, 347.41 μmol) in MeCN (2.5 mL) were added NMI (71.3 mg, 868.52 μmol) and TCFH (121.9 mg, 434.26 μmol) at room temperature. The resulting mixture was stirred for 2 h at room temperature. The resulting mixture was diluted with water (5 mL). The precipitated solids were collected by filtration and washed with water (3×5 mL) to afford tert-butyl N-[1-[7-[[8-(acetamidomethyl)-6-methyl-imidazo[1,2-a]pyrazin-2-yl]carbamoyl]-2-methyl-indazol-4-yl]-4-piperidyl]-N-cyclopropyl-carbamate (100 mg, 162.41 μmol, 56% yield) as a solid. LCMS (ES, m/z): 616 [M+H]⁺.

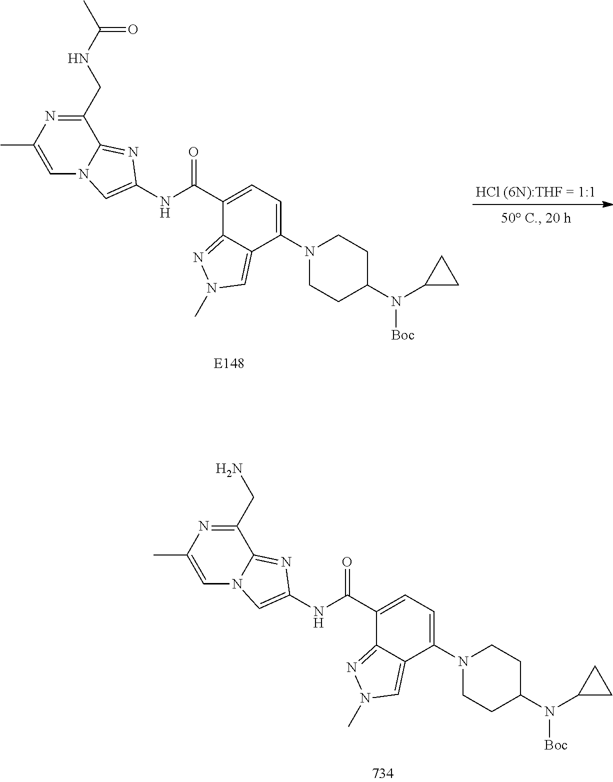 [SMILES: CC(=O)NCc1nc(C)cn2cc(NC(=O)c3ccc(N4CCC(N(C(=O)OC(C)(C)C)C5CC5)CC4)c4cn(C)nc34)nc12 ; Cc1cn2cc(NC(=O)c3ccc(N4CCC(N(C(=O)OC(C)(C)C)C5CC5)CC4)c4cn(C)nc34)nc2c(CN)n1]

To a stirred mixture of tert-butyl N-[1-[7-[[8-(acetamidomethyl)-6-methyl-imidazo[1,2-a]pyrazin-2-yl]carbamoyl]-2-methyl-indazol-4-yl]-4-piperidyl]-N-cyclopropyl-carbamate (40 mg, 64.96 μmol) in THF (0.5 mL) was added HCl (6 N, 0.5 mL) dropwise at room temperature. The resulting mixture was stirred for 20 h at 50° C. The resulting mixture was concentrated under reduced pressure. The crude product was purified by Prep-HPLC with the following conditions (column, C18 silica gel, XBridge, 19×150 mm; mobile phase, MeCN in water (0.05% TFA), 25% to 55% gradient in 7 min; detector, UV 254 nm) to afford N-(8-(aminomethyl)-6-methylimidazo[1,2-a]pyrazin-2-yl)-4-(4-(cyclopropylamino)piperidin-1-yl)-2-methyl-2H-indazole-7-carboxamide bis(2,2,2-trifluoroacetate) (3.3 mg, 4.70 μmol, 7.24% yield) as a solid. LCMS (ES, m/z): 474 [M+H]⁺. ¹H NMR (400 MHz, DMSO-d₆) δ 11.55 (s, 1H), 8.89 (s, 1H), 8.81 (s, 2H), 8.54 (s, 1H), 8.48 (s, 1H), 8.43 (s, 3H), 8.07 (d, J=8.1 Hz, 1H), 6.58 (d, J=8.1 Hz, 1H), 4.58 (d, J=5.2 Hz, 2H), 4.31 (s, 3H), 4.07 (d, J=13.0 Hz, 2H), 3.47-3.39 (m, 1H), 3.07 (t, J=12.5 Hz, 2H), 2.80 (s, 1H), 2.49 (s, 3H), 2.21 (d, J=12.2 Hz, 2H), 1.82-1.68 (m, 2H), 0.85 (d, J=7.4 Hz, 4H).

### Example 263: Synthesis of Compound 735

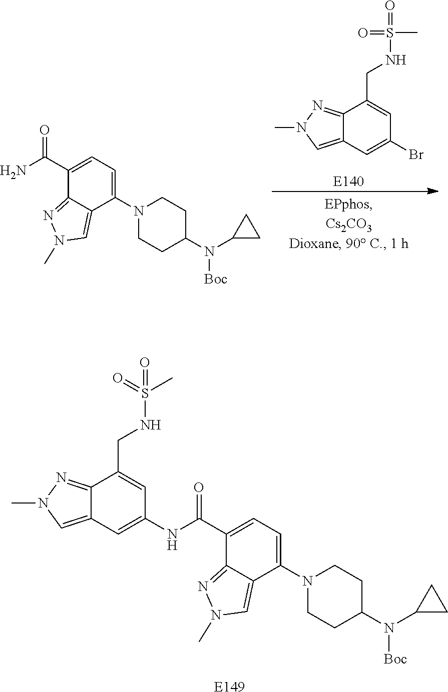

To a stirred mixture of tert-butyl N-[1-(7-carbamoyl-2-methyl-indazol-4-yl)-4-piperidyl]-N-cyclopropyl-carbamate (45.0 mg, 108.82 μmol) and N-[(5-bromo-2-methyl-indazol-7-yl)methyl]methanesulfonamide (34.6 mg, 108.82 μmol) in Dioxane (1 mL) were added Cs₂CO₃ (70.9 mg, 217.65 μmol) and Ephos pd G4 (10.0 mg, 10.88 μmol) at room temperature under nitrogen atmosphere. The resulting mixture was stirred for 1 h at 90° C. under nitrogen atmosphere. The mixture was allowed to cool down to room temperature. The residue was purified by silica gel column chromatography, eluted with DCM/MeOH (15:1) to afford tert-butyl N-cyclopropyl-N-[1-[7-[[7-(methanesulfonamidomethyl)-2-methyl-indazol-5-yl]carbamoyl]-2-methyl-indazol-4-yl]-4-piperidyl]carbamate (50 mg, 76.83 μmol, 71% yield) as a solid. LCMS (ES, m/z): 651 [M+H]⁺.

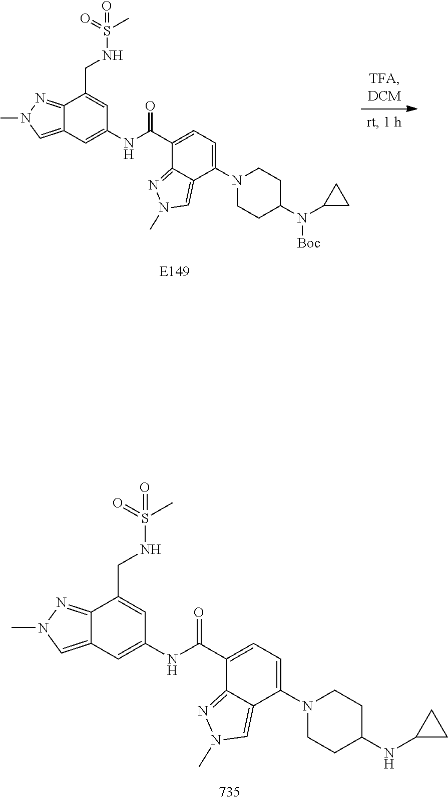

To a solution of tert-butyl N-cyclopropyl-N-[1-[7-[[7-(methanesulfonamidomethyl)-2-methyl-indazol-5-yl]carbamoyl]-2-methyl-indazol-4-yl]-4-piperidyl]carbamate (45.0 mg, 69.15 μmol) in DCM (1 mL) was added TFA (0.4 mL). The reaction was stirred for 1 h at rt. The resulting mixture was concentrated under reduced pressure. The residue was purified by reversed-phase flash chromatography with the following conditions (column, C18 silica gel; mobile phase, CH₃CN in water (0.1% TFA), 25% to 60% gradient in 10 min; detector, UV 254 nm) to afford 4-(4-(cyclopropylamino)piperidin-1-yl)-2-methyl-N-(2-methyl-7-(methylsulfonamidomethyl)-2H-indazol-5-yl)-2H-indazole-7-carboxamide 2,2,2-trifluoroacetate (24.0 mg, 36.11 μmol, 52% yield) as a solid. LCMS (ES, m/z): 551 [M+H]⁺. ¹H NMR (400 MHz, DMSO-d₆) δ 11.15 (s, 1H), 8.85 (s, 3H), 8.33 (s, 1H), 8.25 (d, J=1.9 Hz, 1H), 8.02 (d, J=8.0 Hz, 1H), 7.64 (t, J=6.2 Hz, 1H), 7.43 (s, 1H), 6.56 (d, J=8.1 Hz, 1H), 4.55 (d, J=6.2 Hz, 2H), 4.31 (s, 3H), 4.18 (s, 3H), 4.03 (d, J=12.8 Hz, 2H), 3.48 (s, 1H), 3.01-3.00 (m, 5H), 2.80 (s, 1H), 2.25-2.17 (m, 2H), 1.86-1.71 (m, 2H), 0.85 (d, J=5.5 Hz, 4H).

### Example 264: Synthesis of Compound 736

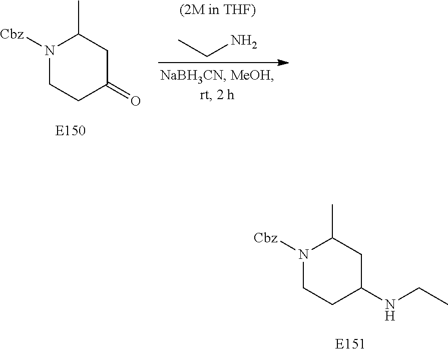

To a solution of benzyl 2-methyl-4-oxo-piperidine-1-carboxylate (2.4 g, 9.71 mmol) in MeOH (25 mL) were added ethanamine (9.71 mL, 19.42 mmol) (2 M in THF) and NaBH₃CN (1.83 g, 29.12 mmol) at 0° C. The mixture was stirred for 2 h at rt. The reaction was diluted with H₂O (100 mL). The resulting mixture was extracted with DCM (2×100 mL). The combined organic layers were washed with H₂O (2×150 mL), dried over anhydrous Na₂SO₄. After filtration, the filtrate was concentrated under reduced pressure to afford benzyl 4-(ethylamino)-2-methyl-piperidine-1-carboxylate (2.5 g, crude) as an oil. LCMS (ES, m/z): 277 [M+H]⁺.

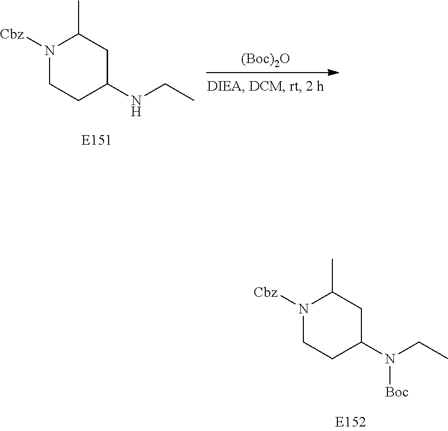 [SMILES: "(Boc)2O" ; CCNC1CCN(C(=O)OCc2ccccc2)C(C)C1 ; CCN(C(=O)OC(C)(C)C)C1CCN(C(=O)OCc2ccccc2)C(C)C1]

To a solution of benzyl 4-(ethylamino)-2-methyl-piperidine-1-carboxylate (2.4 g, 8.68 mmol) in DCM (24 mL) were added DIEA (2.24 g, 17.37 mmol) and tert-butoxycarbonyl tert-butyl carbonate (2.08 g, 9.55 mmol). The mixture was stirred for 2 h at rt. The reaction was diluted with H₂O (50 mL). The resulting mixture was extracted with DCM (2×50 mL). The combined organic layers were washed with H₂O (70 mL), dried over anhydrous Na₂SO₄. After filtration, the filtrate was concentrated under reduced pressure. The residue was purified by silica gel column chromatography, eluted with PE/EA (8/1) to afford benzyl 4-[tert-butoxycarbonyl(ethyl)amino]-2-methyl-piperidine-1-carboxylate (2.4 g, 6.37 mmol, 73% yield) as an oil. LCMS (ES, m/z): 377 [M+H]⁺.

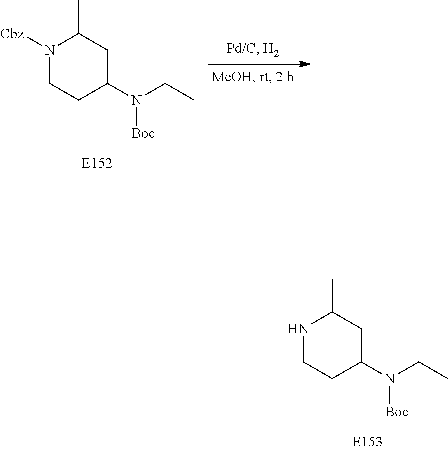 [SMILES: CCN(C(=O)OC(C)(C)C)C1CCN(C(=O)OCc2ccccc2)C(C)C1 ; CCN(C(=O)OC(C)(C)C)C1CCNC(C)C1]

To a solution of benzyl 4-[tert-butoxycarbonyl(ethyl)amino]-2,2-dimethyl-piperidine-1-carboxylate (400 mg, 1.02 mmol) in MeOH (20 mL) was added Pd/C (10% on Carbon (wetted with ca. 55% water)) (163 mg) under N₂. The mixture was stirred for 2 h at rt under H₂. The mixture was filtered through a Celite pad and concentrated under reduced pressure to afford tert-butyl N-(2,2-dimethyl-4-piperidyl)-N-ethyl-carbamate (250 mg, 975.10 μmol, 95% yield) as an oil. LCMS (ES, m/z): 243 [M+H]⁺.

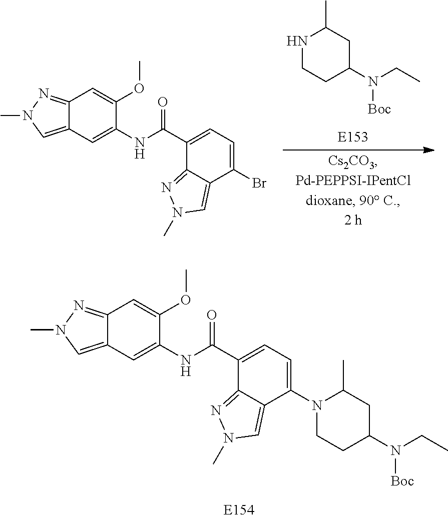

To a stirred mixture of 4-bromo-N-(6-methoxy-2-methyl-indazol-5-yl)-2-methyl-indazole-7-carboxamide (80 mg, 193.12 μmol) in dioxane (1 mL) were added tert-butyl N-ethyl-N-(2-methyl-4-piperidyl)carbamate (56.1 mg, 231.74 μmol), Cs₂CO₃ (125.8 mg, 386.24 μmol) and Pd-PEPPSI-IPentCl (16.2 mg, 19.31 μmol) at rt under N₂. The mixture was stirred for 2 h at 90° C. The reaction was diluted with H₂O (10 mL). The resulting mixture was extracted with DCM (2×10 mL). The combined organic layers were washed with H₂O (10 mL), dried over anhydrous Na₂SO₄. After filtration, the filtrate was concentrated under reduced pressure. The residue was purified by silica gel column chromatography, eluted with DCM/MeOH (19/1) to afford tert-butyl N-ethyl-N-[1-[7-[(6-methoxy-2-methyl-indazol-5-yl)carbamoyl]-2-methyl-indazol-4-yl]-2-methyl-4-piperidyl]carbamate (110 mg, 191.07 μmol, 99% yield) as a solid. LCMS (ES, m/z): 576 [M+H]⁺.

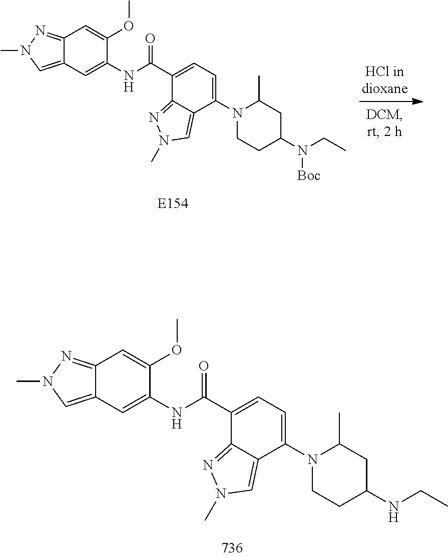

To a solution of tert-butyl N-ethyl-N-[1-[7-[(6-methoxy-2-methyl-indazol-5-yl)carbamoyl]-2-methyl-indazol-4-yl]-2-methyl-4-piperidyl]carbamate (110 mg, 191.07 μmol) in DCM (1.2 mL) was added HCl (4.0 M in 1,4-dioxane) (477 μL) at rt. The mixture was stirred for 2 h at rt. The resulting mixture was concentrated under reduced pressure. The crude product was purified by Prep-HPLC with the following conditions (column, C18 silica gel, XBridge, 19×150 mm; mobile phase, MeCN in water (0.05% TFA), 20% to 35% gradient in 7 min; detector, UV 254 nm) to afford 4-(4-(ethylamino)-2-methylpiperidin-1-yl)-N-(6-methoxy-2-methyl-2H-indazol-5-yl)-2-methyl-2H-indazole-7-carboxamide 2,2,2-trifluoroacetate (27 mg, 45.79 μmol, 24% yield) as a solid. LCMS (ES, m/z): 476 [M+H]⁺. ¹H NMR (400 MHz, DMSO-d₆) δ 11.64 (s, 1H), 8.85 (s, 1H), 8.67 (s, 1H), 8.55 (s, 2H), 8.21 (s, 1H), 8.08 (d, J=7.8 Hz, 1H), 7.09 (s, 1H), 6.82 (d, J=7.9 Hz, 1H), 4.33 (s, 3H), 4.09 (s, 3H), 4.05 (s, 3H), 3.64-3.55 (m, 1H), 3.45 (dd, J=10.8, 4.6 Hz, 1H), 3.36-3.21 (m, 1H), 3.06 (q, J=6.7 Hz, 2H), 2.91 (t, J=11.6 Hz, 1H), 2.24 (d, J=12.4 Hz, 1H), 2.15 (d, J=12.1 Hz, 1H), 1.84-1.72 (m, 1H), 1.57-1.44 (m, 1H), 1.24 (t, J=7.2 Hz, 3H), 1.09 (d, J=5.9 Hz, 3H).

### Example 265: Synthesis of Compound 737

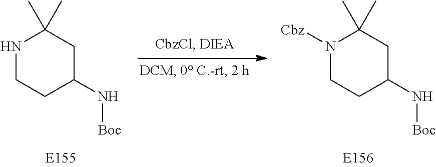 [SMILES: CC1(C)CC(NC(=O)OC(C)(C)C)CCN1 ; CC(C)(C)OC(=O)NC1CCN(C(=O)OCc2ccccc2)C(C)(C)C1]

To a solution of tert-butyl N-(2,2-dimethyl-4-piperidyl)carbamate (500 mg, 2.19 mmol) in DCM (6 mL) was added DIEA (566.0 mg, 4.38 mmol) at rt. Then CbzCl (410.9 mg, 2.41 mmol) was added at 0° C. The mixture was stirred for 2 h at rt. The reaction was diluted with H₂O (30 mL). The resulting mixture was extracted with DCM (2×30 mL). The combined organic layers were washed with H₂O (40 mL), dried over anhydrous Na₂SO₄. After filtration, the filtrate was concentrated under reduced pressure. The residue was purified by silica gel column chromatography, eluted with PE/EA (6/1) to afford benzyl 4-(tert-butoxycarbonylamino)-2,2-dimethyl-piperidine-1-carboxylate (600 mg, 1.66 mmol, 76% yield) as an oil. LCMS (ES, m/z): 363 [M+H]⁺.

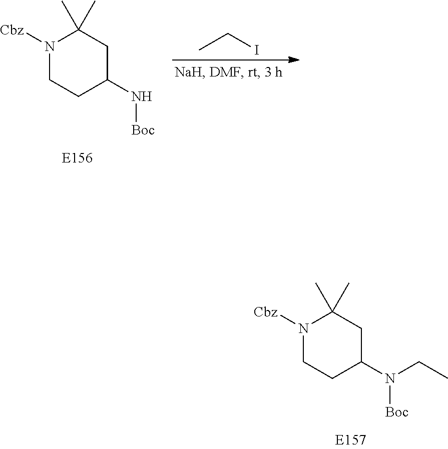

To a solution of benzyl 4-(tert-butoxycarbonylamino)-2,2-dimethyl-piperidine-1-carboxylate (600 mg, 1.66 mmol) in DMF (7 mL) was added NaH (60% dispersion in oil) (132.4 mg, 3.31 mmol, 60% purity) at 0° C. under N₂. The mixture was stirred for 1 h at 0° C. Then iodoethane (387.2 mg, 2.48 mmol) was added at 0° C. under N₂. The mixture was stirred for 2 h at rt. The reaction was diluted with H₂O (30 mL). The resulting mixture was extracted with EA (2×30 mL). The combined organic layers were washed with H₂O (2×30 mL), dried over anhydrous Na₂SO₄. After filtration, the filtrate was concentrated under reduced pressure. The residue was purified by silica gel column chromatography, eluted with PE/EA (10/1) to afford benzyl 4-[tert-butoxycarbonyl(ethyl)amino]-2,2-dimethyl-piperidine-1-carboxylate (640 mg, 1.64 mmol, 99% yield) as an oil. LCMS (ES, m/z): 391 [M+H]⁺.

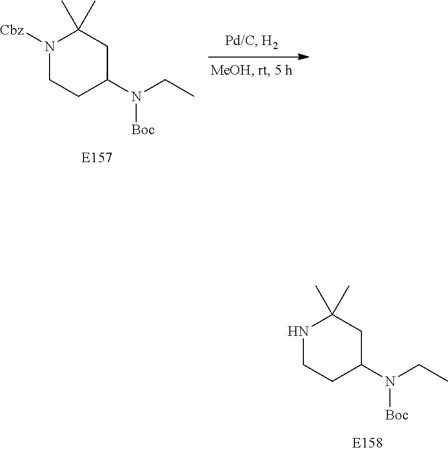 [SMILES: CCN(C(=O)OC(C)(C)C)C1CCN(C(=O)OCc2ccccc2)C(C)(C)C1 ; CCN(C(=O)OC(C)(C)C)C1CCNC(C)(C)C1]

To a solution of benzyl 4-[tert-butoxycarbonyl(ethyl)amino]-2,2-dimethyl-piperidine-1-carboxylate (640 mg, 1.64 mmol) in MeOH (30 mL) was added Pd/C (10% on Carbon (wetted with ca. 55% water)) (226 mg) under N₂. The mixture was stirred for 5 h at rt under H₂. The mixture was filtered through a Celite pad and concentrated under reduced pressure to afford tert-butyl N-(2,2-dimethyl-4-piperidyl)-N-ethyl-carbamate (400 mg, 1.56 mmol, 95% yield) as an oil. LCMS (ES, m/z): 257 [M+H]⁺.

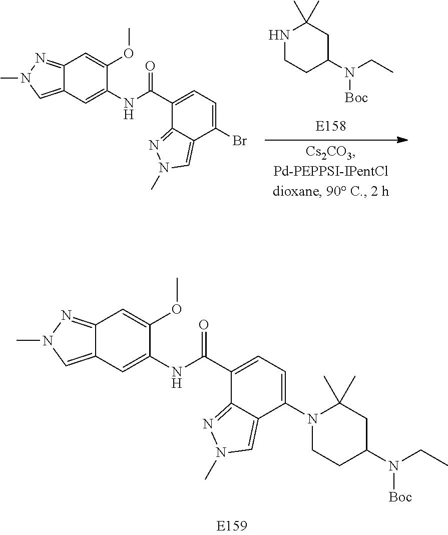 [SMILES: COc1cc2nn(C)cc2cc1NC(=O)c1ccc(Br)c2cn(C)nc12 ; CCN(C(=O)OC(C)(C)C)C1CCNC(C)(C)C1 ; CCN(C(=O)OC(C)(C)C)C1CCN(c2ccc(C(=O)Nc3cc4cn(C)nc4cc3OC)c3nn(C)cc23)C(C)(C)C1]

To a stirred mixture of 4-bromo-N-(6-methoxy-2-methyl-indazol-5-yl)-2-methyl-indazole-7-carboxamide (80 mg, 193.12 μmol) in dioxane (1 mL) were added tert-butyl N-(2,2-dimethyl-4-piperidyl)-N-ethyl-carbamate (59.4 mg, 231.74 μmol), Cs₂CO₃ (125.84 mg, 386.24 μmol) and Pd-PEPPSI-IPentCl (16.2 mg, 19.31 μmol) at rt under N₂. The mixture was stirred for 2 h at 90° C. The reaction was diluted with H₂O (15 mL). The resulting mixture was extracted with DCM (2×15 mL). The combined organic layers were washed with H₂O (15 mL), dried over anhydrous Na₂SO₄. After filtration, the filtrate was concentrated under reduced pressure. The residue was purified by silica gel column chromatography, eluted with DCM/MeOH (20/1) to afford tert-butyl N-ethyl-N-[1-[7-[(6-methoxy-2-methyl-indazol-5-yl)carbamoyl]-2-methyl-indazol-4-yl]-2,2-dimethyl-4-piperidyl]carbamate (105 mg, 178.05 μmol, 92% yield) as a solid. LCMS (ES, m/z): 590 [M+H]⁺.

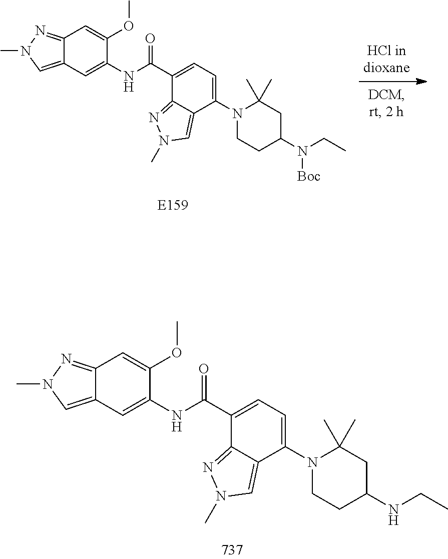

To a solution of tert-butyl N-ethyl-N-[1-[7-[(6-methoxy-2-methyl-indazol-5-yl)carbamoyl]-2-methyl-indazol-4-yl]-2,2-dimethyl-4-piperidyl]carbamate (100 mg, 169.57 μmol) in DCM (1.5 mL) was added HCl (4.0 M in 1,4-dioxane) (423.93 μL) at rt. The mixture was stirred for 2 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 45% to 65% gradient in 7 min; detector, UV 254 nm) to afford 4-[4-(ethylamino)-2,2-dimethyl-1-piperidyl]-N-(6-methoxy-2-methyl-indazol-5-yl)-2-methyl-indazole-7-carboxamide (35 mg, 71.49 μmol, 42% yield) as a solid. LCMS (ES, m/z): 490 [M+H]⁺. ¹H NMR (400 MHz, DMSO-d₆) δ 11.66 (s, 1H), 8.84 (s, 1H), 8.60 (s, 1H), 8.21 (s, 1H), 8.03 (d, J=7.8 Hz, 1H), 7.08 (s, 1H), 6.91 (d, J=7.8 Hz, 1H), 4.31 (s, 3H), 4.09 (s, 3H), 4.05 (s, 3H), 3.49-3.37 (m, 1H), 3.21-3.12 (m, 1H), 2.91-2.85 (m, 1H), 2.65 (p, J=7.0 Hz, 2H), 1.98 (d, J=12.2 Hz, 1H), 1.87-1.78 (m, 1H), 1.38 (dd, J=13.9, 10.0 Hz, 2H), 1.26 (s, 3H), 1.11 (s, 3H), 1.06 (t, J=7.1 Hz, 3H).

### Example 266: Synthesis of Compound 738

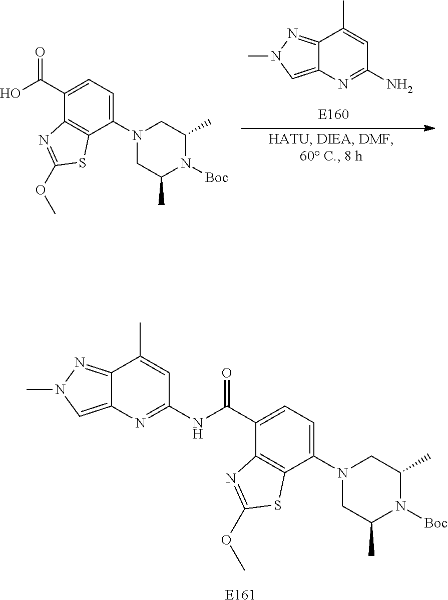

To a stirred mixture of 7-[(3S,5S)-4-tert-butoxycarbonyl-3,5-dimethyl-piperazin-1-yl]-2-methoxy-1,3-benzothiazole-4-carboxylic acid (0.04 g, 94.90 μmol) and 2,7-dimethylpyrazolo[4,3-b]pyridin-5-amine (18.7 mg, 113.88 μmol) in DMF (1 mL) were added DIEA (36.9 mg, 284.69 μmol), HATU (54.2 mg, 142.35 μmol) at room temperature. The resulting mixture was stirred for 8 h at 60° C. under nitrogen atmosphere. The mixture was allowed to cool down to room temperature. The resulting mixture was diluted with water (10 mL). The resulting mixture was extracted with EA (3×3 mL). The combined organic layers were washed with water (2×8 mL), brine (1×8 mL), dried over anhydrous Na₂SO₄. After filtration, the filtrate was concentrated under reduced pressure. The mixture was purified by silica gel column chromatography, eluted with PE/EA (1:6) to afford tert-butyl (2S,6S)-4-(4-((2,7-dimethyl-2H-pyrazolo[4,3-b]pyridin-5-yl)carbamoyl)-2-methoxybenzo[d]thiazol-7-yl)-2,6-dimethylpiperazine-1-carboxylate (0.028 g, 49.50 μmol, 52% yield) as a solid. LCMS (ES, m/z): 566 [M+H]⁺.

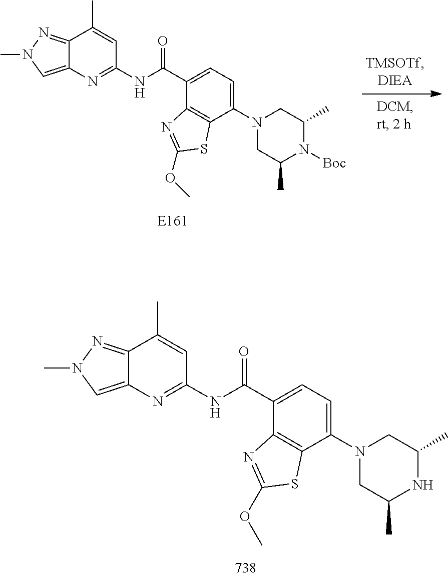

To a solution of tert-butyl (2S,6S)-4-[4-[(2,7-dimethylpyrazolo[4,3-b]pyridin-5-yl)carbamoyl]-2-methoxy-1,3-benzothiazol-7-yl]-2,6-dimethyl-piperazine-1-carboxylate (0.028 g, 49.50 μmol) in DCM (1 mL) was added DIEA (19.2 mg, 148.49 μmol) and TMSOTf (55.1 mg, 247.49 μmol). The reaction was stirred for 2 h at rt. The resulting mixture was concentrated under reduced pressure and adjusted to pH 8 with saturated NaHCO₃ (aq.). After filtration, the filtrate was concentrated under reduced pressure. The solution was purified by reversed-phase flash chromatography with the following conditions (SunFire Prep C18 OBD Column 19*150 mm, 5 μm 10 nm, mobile phase, MeCN in water (0.05% TFA), 10% to 30% gradient in 7 min; detector, UV 254 nm) to afford N-(2,7-dimethyl-2H-pyrazolo[4,3-b]pyridin-5-yl)-7-((3S,5S)-3,5-dimethylpiperazin-1-yl)-2-methoxybenzo[d]thiazole-4-carboxamide 2,2,2-trifluoroacetate (0.01 g, 17.25 μmol, 35% yield) as a solid. LCMS (ES, m/z): 466 [M+H]⁺. ¹H NMR (400 MHz, Methanol-d₄) δ 8.32 (d, J=8.5 Hz, 1H), 8.28 (s, 1H), 7.96 (s, 1H), 7.19 (d, J=8.6 Hz, 1H), 4.49 (s, 3H), 4.28 (s, 3H), 3.89 (s, 2H), 3.93-3.84 (m, 2H), 3.48 (dd, J=13.2, 3.3 Hz, 2H), 2.70 (s, 3H), 1.56 (d, J=6.6 Hz, 6H).

### Example 267: Synthesis of Compound 739

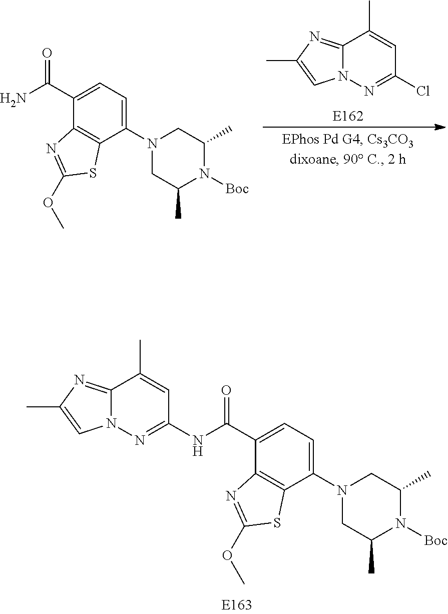 [SMILES: COc1nc2c(C(N)=O)ccc(N3C[C@H](C)N(C(=O)OC(C)(C)C)[C@@H](C)C3)c2s1 ; Cc1cn2nc(Cl)cc(C)c2n1 ; COc1nc2c(C(=O)Nc3cc(C)c4nc(C)cn4n3)ccc(N3C[C@H](C)N(C(=O)OC(C)(C)C)[C@@H](C)C3)c2s1]

To a stirred mixture of tert-butyl (2S,6S)-4-(4-carbamoyl-2-methoxy-1,3-benzothiazol-7-yl)-2,6-dimethyl-piperazine-1-carboxylate (40 mg, 95.12 μmol) and 6-chloro-2,8-dimethyl-imidazo[1,2-b]pyridazine (20.7 mg, 114.14 μmol) in Dioxane (1 mL) were added Cs₂CO₃ (93.0 mg, 285.36 μmol) and EPhos Pd G4 (8.7 mg, 9.51 μmol) at room temperature under nitrogen atmosphere. The resulting mixture was stirred for 2 h at 90° C. under nitrogen atmosphere. The resulting mixture was concentrated under reduced pressure. The residue was purified by silica gel column chromatography, eluted with DCM/MeOH (10:1) to afford tert-butyl (2S,6S)-4-[4-[(2,8-dimethylimidazo[1,2-b]pyridazin-6-yl)carbamoyl]-2-methoxy-1,3-benzothiazol-7-yl]-2,6-dimethyl-piperazine-1-carboxylate (50 mg, 88.39 μmol, 93% yield) as a solid. LCMS (ES, m/z): 566 [M+H]⁺.

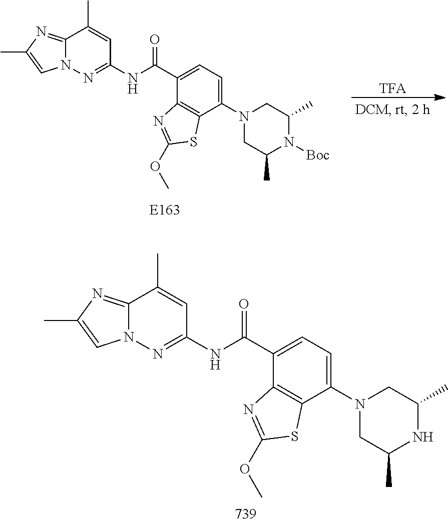

To a stirred mixture of tert-butyl (2S,6S)-4-[4-[(2,8-dimethylimidazo[1,2-b]pyridazin-6-yl)carbamoyl]-2-methoxy-1,3-benzothiazol-7-yl]-2,6-dimethyl-piperazine-1-carboxylate (50 mg, 88.39 μmol) in DCM (1 mL) was added TFA (0.5 mL) dropwise at room temperature. The resulting mixture was stirred for 2 h at room temperature. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 30% to 80% gradient in 7 min; detector, UV 254 nm) to afford N-(2,8-dimethylimidazo[1,2-b]pyridazin-6-yl)-7-[(3S,5S)-3,5-dimethylpiperazin-1-yl]-2-methoxy-1,3-benzothiazole-4-carboxamide (12.5 mg, 26.85 μmol, 30% yield) as a solid. LCMS (ES, m/z): 466 [M+H]⁺. ¹H NMR (400 MHz, DMSO-d₆) δ 12.13 (s, 1H), 8.15 (d, J=8.5 Hz, 1H), 8.04 (s, 1H), 7.86 (s, 1H), 7.06 (d, J=8.6 Hz, 1H), 4.38 (s, 3H), 3.25 (dq, J=6.6, 3.1 Hz, 2H), 3.15 (dd, J=11.7, 3.1 Hz, 2H), 2.95 (dd, J=11.6, 6.1 Hz, 2H), 2.57 (s, 3H), 2.37 (s, 3H), 1.17 (d, J=6.4 Hz, 6H).

### Example 268: Synthesis of Compound 740

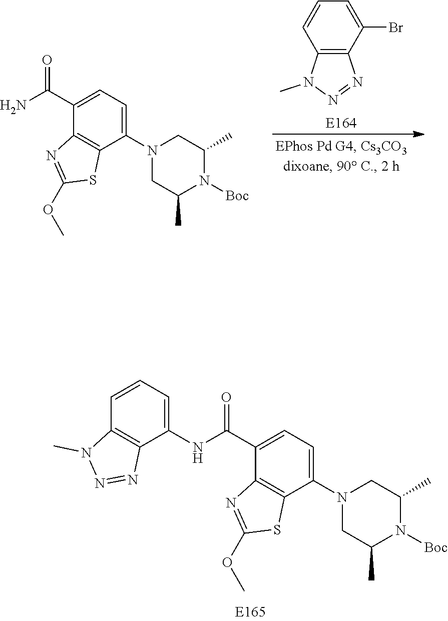 [SMILES: COc1nc2c(C(N)=O)ccc(N3C[C@H](C)N(C(=O)OC(C)(C)C)[C@@H](C)C3)c2s1 ; Cn1nnc2c(Br)cccc21 ; COc1nc2c(C(=O)Nc3cccc4c3nnn4C)ccc(N3C[C@H](C)N(C(=O)OC(C)(C)C)[C@@H](C)C3)c2s1]

To a stirred mixture of tert-butyl (2S,6S)-4-(4-carbamoyl-2-methoxy-1,3-benzothiazol-7-yl)-2,6-dimethyl-piperazine-1-carboxylate (0.055 g, 130.79 μmol) and 4-bromo-1-methyl-benzotriazole (33.8 mg, 156.95 μmol) in Dioxane (1 mL) were added Cs₂CO₃ (85.3 mg, 261.58 μmol), EPhos Pd G4 (12.1 mg, 13.08 μmol) at room temperature under nitrogen atmosphere. The resulting mixture was stirred for 2 h at 90° C. under nitrogen atmosphere. The mixture was allowed to cool down to room temperature. After filtration, the filtrate was concentrated under reduced pressure. The mixture was purified by silica gel column chromatography, eluted with PE/EA (1:3) to afford tert-butyl (2S,6S)-4-[2-methoxy-4-[(1-methylbenzotriazol-4-yl)carbamoyl]-1,3-benzothiazol-7-yl]-2,6-dimethyl-piperazine-1-carboxylate (0.062 g, 112.39 μmol, 86% yield) as a solid. LCMS (ES, m/z): 552 [M+H]⁺.

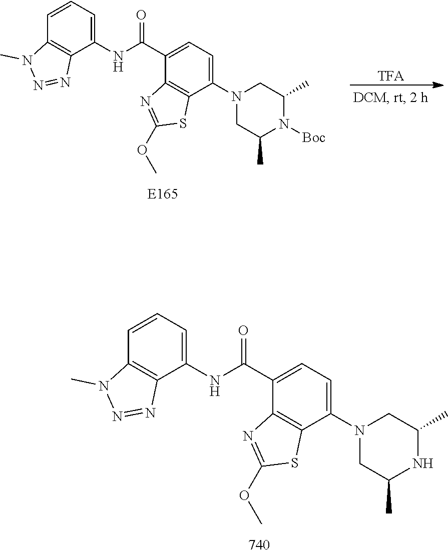

To a solution of tert-butyl (2S,6S)-4-[2-methoxy-4-[(1-methylbenzotriazol-4-yl)carbamoyl]-1,3-benzothiazol-7-yl]-2,6-dimethyl-piperazine-1-carboxylate (0.062 g, 112.39 μmol) in DCM (1 mL) was added TFA (0.4 mL). The reaction was stirred for 2 h at rt. The resulting mixture was concentrated under reduced pressure and adjusted to pH 8 with saturated NaHCO₃ (aq.). After filtration, the filtrate was concentrated under reduced pressure. The residue was purified by reversed-phase flash chromatography with the following conditions (column, C18 silica gel; mobile phase, CH₃CN in water (0.1% NH₃·H₂O), 25% to 60% gradient in 10 min; detector, UV 254 nm) to afford 7-[(3S,5S)-3,5-dimethylpiperazin-1-yl]-2-methoxy-N-(1-methylbenzotriazol-4-yl)-1,3-benzothiazole-4-carboxamide (0.015 g, 33.22 μmol, 30% yield) as a solid. LCMS (ES, m/z): 452 [M+H]⁺. ¹H NMR (400 MHz, DMSO-d₆) δ 12.60 (s, 1H), 8.45 (d, J=7.2 Hz, 1H), 8.19 (d, J=8.5 Hz, 1H), 7.53 (t, J=7.8 Hz, 1H), 7.49 (d, J=8.1 Hz, 1H), 7.04 (d, J=8.6 Hz, 1H), 4.63 (s, 3H), 4.30 (s, 3H), 3.21 (tt, J=9.2, 4.7 Hz, 2H), 3.11 (dd, J=11.6, 3.1 Hz, 2H), 2.93 (dd, J=11.5, 6.1 Hz, 2H), 1.15 (d, J=6.4 Hz, 6H).

### Example 269: Synthesis of Compound 742

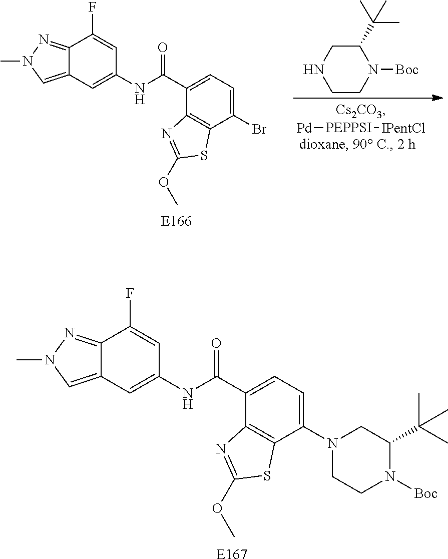

To a stirred mixture of 7-bromo-N-(7-fluoro-2-methyl-indazol-5-yl)-2-methoxy-1,3-benzothiazole-4-carboxamide (60 mg, 137.85 μmol) in dioxane (0.7 mL) were added tert-butyl (2S)-2-tert-butylpiperazine-1-carboxylate (40.1 mg, 165.41 μmol), Cs₂CO₃ (89.8 mg, 275.69 μmol) and Pd-PEPPSI-IPentCl (11.6 mg, 13.78 μmol) at rt under N₂. The mixture was stirred for 2 h at 90° C. The reaction was diluted with H₂O (10 mL). The resulting mixture was extracted with DCM (2×10 mL). The combined organic layers were washed with H₂O (10 mL), dried over anhydrous Na₂SO₄. After filtration, the filtrate was concentrated under reduced pressure. The residue was purified by silica gel column chromatography, eluted with DCM/EA (4/1) to afford tert-butyl(2S)-2-tert-butyl-4-[4-[(7-fluoro-2-methyl-indazol-5-yl)carbamoyl]-2-methoxy-1,3-benzothiazol-7-yl]piperazine-1-carboxylate (65 mg, 108.93 μmol, 79% yield) as a solid. LCMS (ES, m/z): 597 [M+H]⁺.

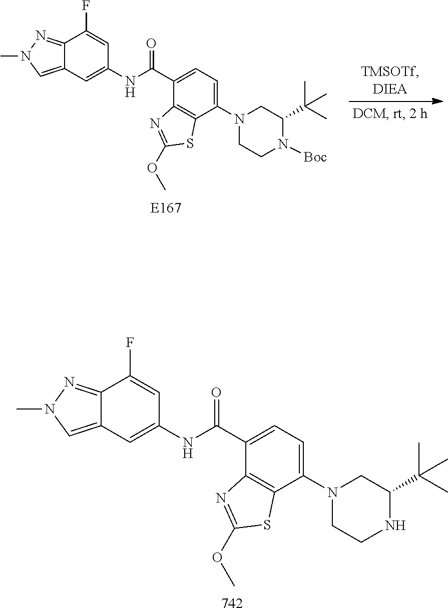

To a solution of tert-butyl(2S)-2-tert-butyl-4-[4-[(7-fluoro-2-methyl-indazol-5-yl)carbamoyl]-2-methoxy-1,3-benzothiazol-7-yl]piperazine-1-carboxylate (60 mg, 100.55 μmol) in DCM (0.6 mL) were added DIEA (26.0 mg, 201.10 μmol) and TMSOTf (67.0 mg, 301.65 μmol). The mixture was stirred for 2 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 40% to 70% gradient in 7 min; detector, UV 254 nm) to afford 7-[(3S)-3-tert-butylpiperazin-1-yl]-N-(7-fluoro-2-methyl-indazol-5-yl)-2-methoxy-1,3-benzothiazole-4-carboxamide (15 mg, 30.21 μmol, 30% yield) as a solid. LCMS (ES, m/z): 497 [M+H]⁺. ¹H NMR (400 MHz, DMSO-d₆) δ 11.41 (s, 1H), 8.44 (d, J=2.8 Hz, 1H), 8.16-8.08 (m, 2H), 7.36 (dd, J=13.1, 1.6 Hz, 1H), 7.09 (d, J=8.6 Hz, 1H), 4.41 (s, 3H), 4.19 (s, 3H), 3.60 (d, J=10.9 Hz, 1H), 3.37 (s, 1H), 3.09 (d, J=9.1 Hz, 1H), 2.83 (q, J=9.6, 7.1 Hz, 2H), 2.57-2.52 (m, 1H), 2.45 (d, J=10.5 Hz, 1H), 0.95 (s, 9H).

### Example 270: Synthesis of Compound 743

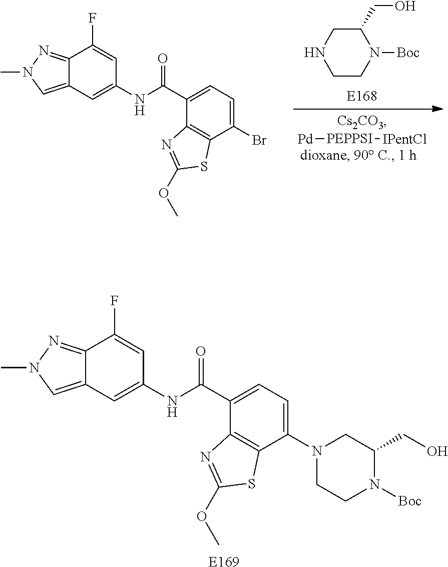

To a stirred mixture of 7-bromo-N-(7-fluoro-2-methyl-indazol-5-yl)-2-methoxy-1,3-benzothiazole-4-carboxamide (0.1 g, 229.74 μmol) and tert-butyl(2R)-2-(hydroxymethyl) piperazine-1-carboxylate (59.6 mg, 275.69 μmol) in Dioxane (1 mL) were added Cs₂CO₃ (149.7 mg, 459.49 μmol), Pd-PEPPSI™-IPent catalyst (18.2 mg, 22.97 μmol) at room temperature under nitrogen atmosphere. The resulting mixture was stirred for 1 h at 90° C. under nitrogen atmosphere. The mixture was allowed to cool down to room temperature. After filtration, the filtrate was concentrated under reduced pressure. The mixture was purified by silica gel column chromatography, eluted with PE/EA (1:3) to afford tert-butyl(2R)-4-[4-[(7-fluoro-2-methyl-indazol-5-yl)carbamoyl]-2-methoxy-1,3-benzothiazol-7-yl]-2-(hydroxymethyl) piperazine-1-carboxylate (0.04 g, 70.10 μmol, 31% yield) as a solid. LCMS (ES, m/z): 571 [M+H]⁺.

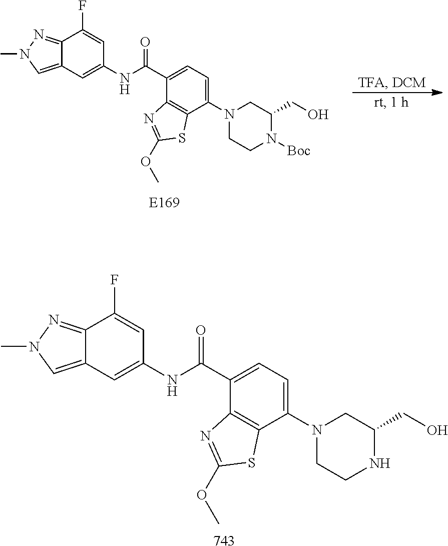

To a solution of tert-butyl(2R)-4-[4-[(7-fluoro-2-methyl-indazol-5-yl)carbamoyl]-2-methoxy-1,3-benzothiazol-7-yl]-2-(hydroxymethyl) piperazine-1-carboxylate (0.04 g, 70.10 μmol) in DCM (1 mL) was added TFA (0.4 mL). The reaction was stirred for 1 h at rt. The resulting mixture was concentrated under reduced pressure and adjusted to pH 8 with saturated NaHCO₃ (aq.). After filtration, the filtrate was concentrated under reduced pressure. The residue was purified by reversed-phase flash chromatography with the following conditions (SunFire Prep C18 OBD Column 19*150 mm, 5 μm 10 nm, mobile phase, MeCN in water (0.05% TFA), 10% to 30% gradient in 7 min; detector, UV 254 nm) to afford (R)—N-(7-fluoro-2-methyl-2H-indazol-5-yl)-7-(3-(hydroxymethyl) piperazin-1-yl)-2-methoxybenzo[d]thiazole-4-carboxamide 2,2,2-trifluoroacetate (0.015 g, 25.66 μmol, 37% yield) as a solid. LCMS (ES, m/z): 471 [M+H]⁺. ¹H NMR (400 MHz, DMSO-d₆) δ 11.38 (s, 1H), 9.25-9.15 (m, 1H), 8.90-8.75 (m, 1H), 8.44 (d, J=2.8 Hz, 1H), 8.17 (d, J=8.4 Hz, 1H), 8.11 (s, 1H), 7.36 (d, J=13.0 Hz, 1H), 7.19 (d, J=8.5 Hz, 1H), 5.53 (d, J=5.3 Hz, 1H), 4.43 (s, 3H), 4.20 (s, 3H), 3.77-3.62 (m, 4H), 3.50-3.40 (m, 2H), 3.26 (d, J=12.1 Hz, 1H), 3.17-3.03 (m, 2H).

### Example 271: Synthesis of Compound 744

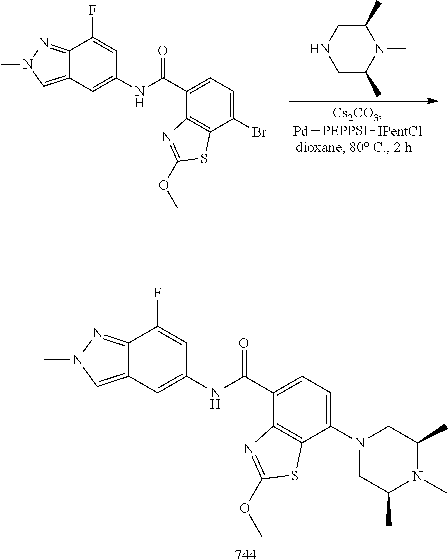

To a stirred mixture of 7-bromo-N-(7-fluoro-2-methyl-indazol-5-yl)-2-methoxy-1,3-benzothiazole-4-carboxamide (90 mg, 206.77 μmol) in dioxane (1 mL) were added (2R,6S)-1,2,6-trimethylpiperazine (31.8 mg, 248.12 μmol), Cs₂CO₃ (134.7 mg, 413.54 μmol) and Pd-PEPPSI-IPentCl (17.4 mg, 20.69 μmol) at rt under N₂. The mixture was stirred for 2 h at 80° C. under N₂. The reaction was diluted with H₂O (10 mL). The resulting mixture was extracted with DCM (2×10 mL). The combined organic layers were washed with H₂O (20 mL), dried over anhydrous Na₂SO₄. After filtration, the filtrate was concentrated under reduced pressure. The residue was purified by silica gel column chromatography, eluted with DCM/MeOH (19/1). After concentrated, the product was triturated with MeOH to afford N-(7-fluoro-2-methyl-indazol-5-yl)-2-methoxy-7-[(3R,5S)-3,4,5-trimethylpiperazin-1-yl]-1,3-benzothiazole-4-carboxamide (30 mg, 62.17 μmol, 30% yield) as a solid. LCMS (ES, m/z): 483 [M+H]⁺. ¹H NMR (400 MHz, DMSO-d₆) δ 11.37 (s, 1H), 8.41 (d, J=2.8 Hz, 1H), 8.10 (d, J=8.5 Hz, 1H), 8.06 (d, J=1.6 Hz, 1H), 7.32 (dd, J=13.1, 1.6 Hz, 1H), 7.03 (d, J=8.6 Hz, 1H), 4.38 (s, 3H), 4.17 (s, 3H), 3.42 (d, J=11.6 Hz, 2H), 2.62 (t, J=11.2 Hz, 2H), 2.30 (ddd, J=10.1, 6.1, 2.7 Hz, 2H), 2.21 (s, 3H), 1.06 (d, J=6.0 Hz, 6H).

### Example 272: Synthesis of Compound 745

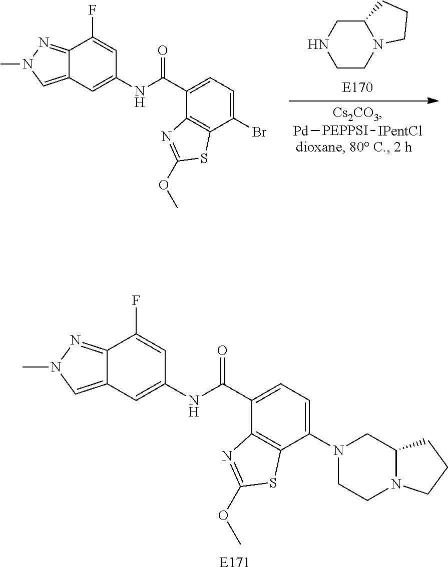

To a stirred mixture of 7-bromo-N-(7-fluoro-2-methyl-indazol-5-yl)-2-methoxy-1,3-benzothiazole-4-carboxamide (100 mg, 229.74 μmol) in dioxane (0.1 mL) were added (8aS)-1,2,3,4,6,7,8,8a-octahydropyrrolo[1,2-a]pyrazine (34.8 mg, 275.69 μmol), Cs₂CO₃ (149.7 mg, 459.49 μmol) and Pd-PEPPSI-IPentCl (19.3 mg, 22.97 μmol) at rt under N₂. The mixture was stirred for 2 h at 80° C. under N₂. The reaction was diluted with H₂O (10 mL). The resulting mixture was extracted with DCM (2×10 mL). The combined organic layers were washed with H₂O (10 mL), dried over anhydrous Na₂SO₄. After filtration, the filtrate was concentrated under reduced pressure. The residue was purified by silica gel column chromatography, eluted with DCM/MeOH (19/1). After concentrated, the product was purified by Prep-HPLC with the following conditions (column, C18 silica gel, XBridge, 19×150 mm; mobile phase, MeCN in water (0.05% NH₃H₂O), 25% to 45% gradient in 7 min; detector, UV 254 nm) to afford 7-[(8aS)-3,4,6,7,8,8a-hexahydro-1H-pyrrolo[1,2-a]pyrazin-2-yl]-N-(7-fluoro-2-methyl-indazol-5-yl)-2-methoxy-1,3-benzothiazole-4-carboxamide (11 mg, 22.89 μmol, 10% yield) as a solid. LCMS (ES, m/z): 481 [M+H]⁺. ¹H NMR (400 MHz, DMSO-d₆) δ 11.38 (s, 1H), 8.41 (d, J=2.8 Hz, 1H), 8.14-8.05 (m, 2H), 7.33 (dd, J=13.1, 1.6 Hz, 1H), 7.09 (d, J=8.6 Hz, 1H), 4.39 (s, 3H), 4.17 (s, 3H), 3.63 (d, J=11.1 Hz, 1H), 3.52 (d, J=11.7 Hz, 1H), 3.11-2.90 (m, 3H), 2.66 (t, J=10.6 Hz, 1H), 2.30 (dd, J=11.3, 8.3 Hz, 1H), 2.12 (q, J=8.7 Hz, 2H), 1.81 (d, J=6.4 Hz, 1H), 1.78-1.62 (m, 2H), 1.43-1.32 (m, 1H).

### Example 273: Synthesis of Compound 747

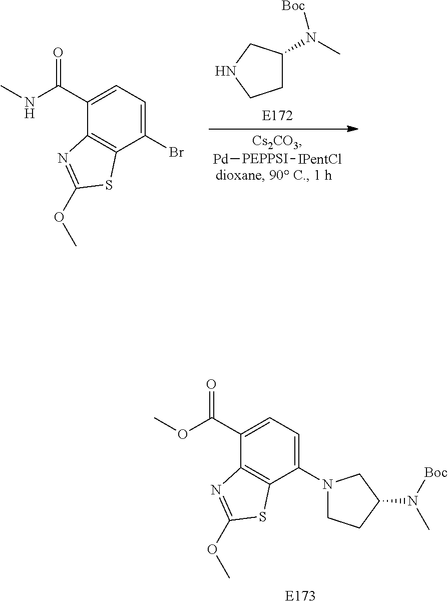

To a stirred mixture of methyl 7-bromo-2-methoxy-1,3-benzothiazole-4-carboxylate (0.11 g, 364.06 μmol) and tert-butyl N-methyl-N-[(3R)-pyrrolidin-3-yl]carbamate (87.5 mg, 436.88 μmol) in Dioxane (2 mL) were added Cs₂CO₃ (237.4 mg, 728.13 μmol), Pd-PEPPSI™-IPent catalyst (28.8 mg, 36.41 μmol) at room temperature under nitrogen atmosphere. The resulting mixture was stirred for 1 h at 90° C. under nitrogen atmosphere. The mixture was allowed to cool down to room temperature. After filtration, the filtrate was concentrated under reduced pressure. The mixture was purified by silica gel column chromatography, eluted with PE/EA (1:1) to afford methyl 7-[(3R)-3-[tert-butoxycarbonyl(methyl)amino]pyrrolidin-1-yl]-2-methoxy-1,3-benzothiazole-4-carboxylate (0.13 g, 308.42 μmol, 85% yield) as a solid. LCMS (ES, m/z): 422 [M+H]⁺.

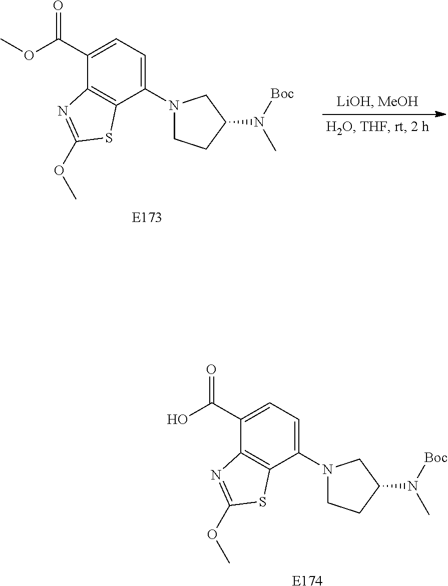 [SMILES: COC(=O)c1ccc(N2CC[C@@H](N(C)C(=O)OC(C)(C)C)C2)c2sc(OC)nc12 ; "E173" ; COc1nc2c(C(=O)O)ccc(N3CC[C@@H](N(C)C(=O)OC(C)(C)C)C3)c2s1 ; "E174"]

To a stirred mixture of methyl 7-[(3R)-3-[tert-butoxycarbonyl(methyl)amino]pyrrolidin-1-yl]-2-methoxy-1,3-benzothiazole-4-carboxylate (0.12 g, 284.69 μmol) in MeOH (1 mL), THF (1 mL) and H₂O (0.5 mL) was added LiOH (34.09 mg, 1.42 mmol) at room temperature. The resulting mixture was stirred for 2 h at room temperature. The mixture residue was neutralized to pH 5 with 1 N of HCl (aq.). The resulting mixture was diluted with water (3 mL). The resulting mixture was extracted with EA (3×2 mL). The combined organic layers were washed with water (1×4 mL), brine (1×4 mL), dried over anhydrous Na₂SO₄. After filtration, the filtrate was concentrated under reduced pressure to afford 7-[(3R)-3-[tert-butoxycarbonyl(methyl)amino]pyrrolidin-1-yl]-2-methoxy-1,3-benzothiazole-4-carboxylic acid (0.1 g, 245.41 μmol, 86% yield) as a solid. LCMS (ES, m/z): 408 [M+H]⁺.

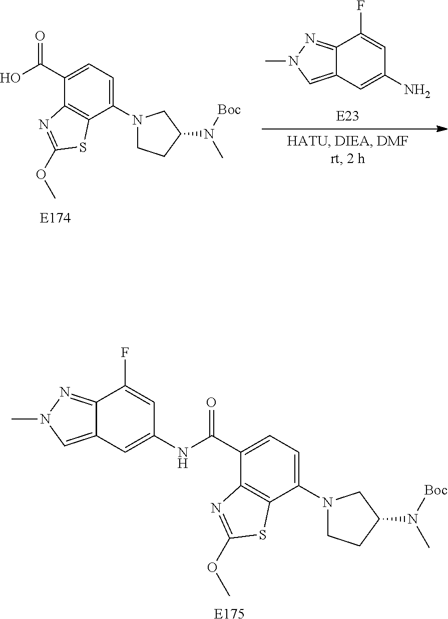

To a stirred mixture of 7-[(3R)-3-[tert-butoxycarbonyl(methyl)amino]pyrrolidin-1-yl]-2-methoxy-1,3-benzothiazole-4-carboxylic acid (0.1 g, 245.41 μmol) and 7-fluoro-2-methyl-indazol-5-amine (48.6 mg, 294.49 μmol) in DMF (2 mL) were added DIEA (95.5 mg, 736.23 μmol), HATU (139.9 mg, 368.11 μmol) at room temperature. The resulting mixture was stirred for 2 h at room temperature. The resulting mixture was diluted with water (3 mL). The resulting mixture was extracted with EA (3×3 mL). The combined organic layers were washed with water (1×8 mL), brine (1×8 mL), dried over anhydrous Na₂SO₄. After filtration, the filtrate was concentrated under reduced pressure to afford tert-butyl N-[(3R)-1-[4-[(7-fluoro-2-methyl-indazol-5-yl)carbamoyl]-2-methoxy-1,3-benzothiazol-7-yl]pyrrolidin-3-yl]-N-methyl-carbamate (0.09 g, 162.27 μmol, 66% yield) as a solid. LCMS (ES, m/z): 555 [M+H]⁺.

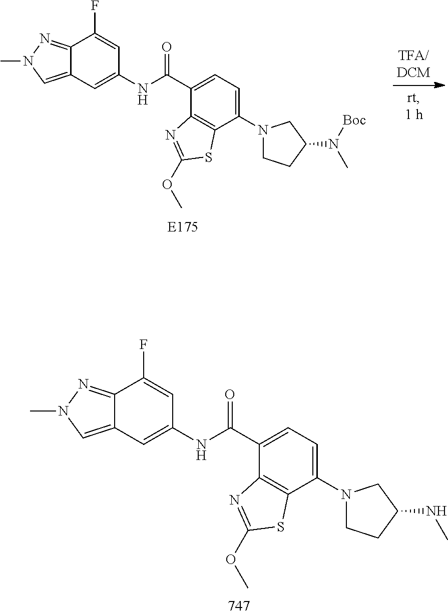

To a solution of tert-butyl N-[(3R)-1-[4-[(7-fluoro-2-methyl-indazol-5-yl)carbamoyl]-2-methoxy-1,3-benzothiazol-7-yl]pyrrolidin-3-yl]-N-methyl-carbamate (0.09 g, 162.27 μmol) in DCM (1 mL) was added TFA (0.4 mL). The reaction was stirred for 1 h at rt. The resulting mixture was concentrated under reduced pressure and adjusted to pH 8 with saturated NaHCO₃ (aq.). After filtration, the filtrate was concentrated under reduced pressure. The residue was purified by reversed-phase flash chromatography with the following conditions (column, C18 silica gel; mobile phase, CH₃CN in water (0.1% NH₃·H₂O), 45% to 75% gradient in 10 min; detector, UV 254 nm) to afford N-(7-fluoro-2-methyl-indazol-5-yl)-2-methoxy-7-[(3R)-3-(methylamino)pyrrolidin-1-yl]-1,3-benzothiazole-4-carboxamide (0.03 g, 66.00 μmol, 41% yield) as a solid. LCMS (ES, m/z): 455 [M+H]⁺ Chiral HPLC: Retention Time: 2.836 min 1H NMR (400 MHz, DMSO-d₆) δ 11.46 (s, 1H), 8.41 (s, 1H), 8.08-7.98 (m, 2H), 7.34 (d, J=13.1 Hz, 1H), 6.62-6.54 (m, 1H), 4.43-4.37 (m, 3H), 4.19 (d, J=3.2 Hz, 3H), 3.77 (s, 2H), 3.71 (s, 1H), 3.63-3.60 (m, 2H), 2.37 (d, J=3.1 Hz, 3H), 2.19-2.11 (m, 1H), 1.93-1.85 (m, 1H).

### Example 274: Synthesis of Compound 748

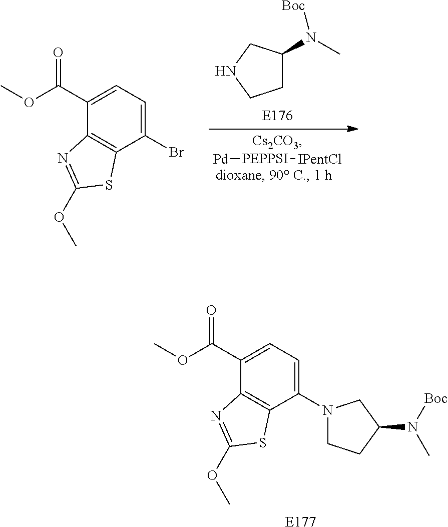

To a stirred mixture of methyl 7-bromo-2-methoxy-1,3-benzothiazole-4-carboxylate (100.0 mg, 330.97 μmol) and tert-butyl N-(azetidin-3-yl)-N-methyl-carbamate (123.2 mg, 661.94 μmol) in dioxane (2 mL) were added Cs₂CO₃ (161.7 mg, 496.45 μmol), Pd-PEPPSI-IPentCl (27.7 mg, 33.10 μmol) at room temperature under nitrogen atmosphere. The resulting mixture was stirred for 1 h at 90° C. under nitrogen atmosphere. The mixture was allowed to cool down to room temperature. The resulting mixture was diluted with water (10 mL). The resulting mixture was extracted with EA (2×5 mL). The combined organic layers were washed with water (2×5 mL), brine (1×5 mL), dried over anhydrous Na₂SO₄. After filtration, the filtrate was concentrated under reduced pressure. The residue was purified by silica gel column chromatography, eluted with PE/EA (2:1) to afford methyl 7-[3-[tert-butoxycarbonyl(methyl)amino]azetidin-1-yl]-2-methoxy-1,3-benzothiazole-4-carboxylate (80 mg, 196.33 μmol, 59% yield) as a solid. LCMS (ES, m/z): 422 [M+H]⁺.

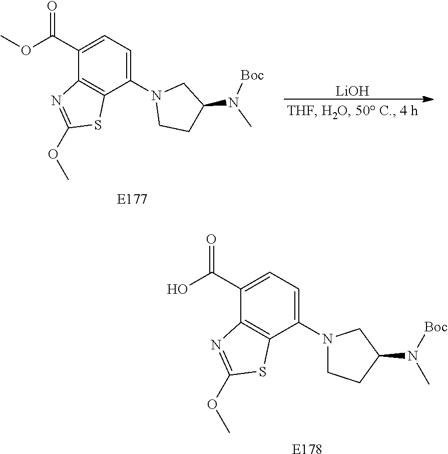

To a stirred solution of methyl 7-[(3S)-3-[tert-butoxycarbonyl(methyl)amino]pyrrolidin-1-yl]-2-methoxy-1,3-benzothiazole-4-carboxylate (100.0 mg, 237.24 μmol) in THF (1 mL), MeOH (1 mL) was added LiOH (39.7 mg, 1.66 mmol) in portions at rt. The resulting mixture was stirred for 4 h at 50° C. The resulting mixture was diluted with water (20 ml). The mixture was acidified to pH 6 with HCl (2N). The resulting mixture was extracted with EA (2×20 mL). The combined organic layers were washed with brine (1×20 mL), dried over anhydrous Na₂SO₄. After filtration, the filtrate was concentrated under reduced pressure. This resulted in 7-[(3S)-3-[tert-butoxycarbonyl(methyl)amino]pyrrolidin-1-yl]-2-methoxy-1,3-benzothiazole-4-carboxylic acid (80 mg, 196.33 μmol, 83% yield) as a solid. LCMS (ES, m/z): 408 [M+H]⁺.

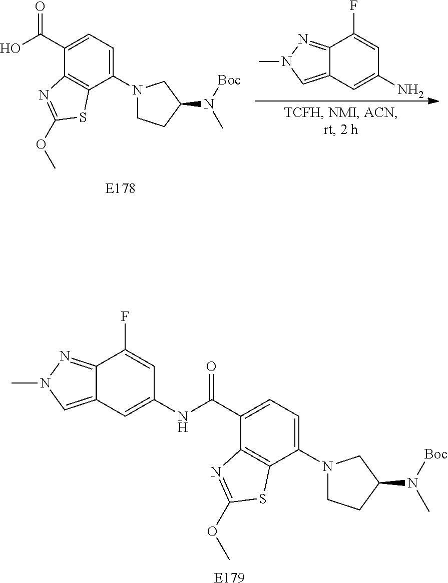

To a stirred mixture of 7-[(3S)-3-[tert-butoxycarbonyl(methyl)amino]pyrrolidin-1-yl]-2-methoxy-1,3-benzothiazole-4-carboxylic acid (75.0 mg, 184.06 μmol) and 7-fluoro-2-methyl-indazol-5-amine (33.4 mg, 202.46 μmol) in MeCN (1.5 mL) was added NMI (75.5 mg, 920.28 μmol) and TCFH (72.3 mg, 257.68 μmol) at room temperature. The resulting mixture was stirred for 2 h at rt. The resulting mixture was diluted with water (3 mL). The precipitated solids were collected by filtration and washed with MeCN (1 mL). This resulted in tert-butyl N-[(3S)-1-[4-[(7-fluoro-2-methyl-indazol-5-yl)carbamoyl]-2-methoxy-1,3-benzothiazol-7-yl]pyrrolidin-3-yl]-N-methyl-carbamate (80 mg, 144.24 μmol, 78% yield) as a solid. LCMS (ES, m/z): 555 [M+H]⁺.

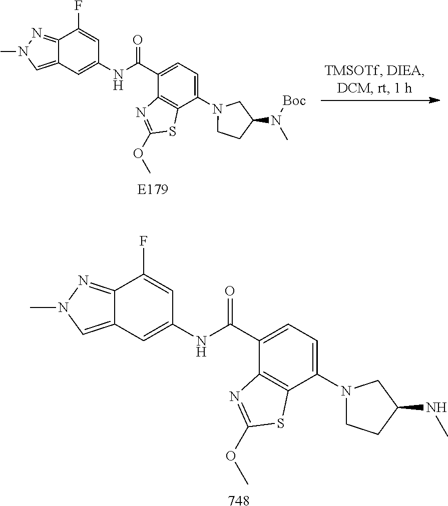

To a solution of tert-butyl N-[(3S)-1-[4-[(7-fluoro-2-methyl-indazol-5-yl)carbamoyl]-2-methoxy-1,3-benzothiazol-7-yl]pyrrolidin-3-yl]-N-methyl-carbamate (70.0 mg, 126.21 μmol) in DCM (1 mL) was added DIEA (32.6 mg, 252.42 μmol) and TMSOTf (84.1 mg, 378.63 μmol). The reaction was stirred for 1 h at rt. The resulting mixture was concentrated under reduced pressure and adjusted to pH 8 with saturated NaHCO₃ (aq.). The resulting mixture was diluted with water (5 mL) and extracted with DCM/MeOH (20/1) (2×5 mL). The combined organic layers were washed with water (1×5 mL), brine (1×5 mL) and dried over anhydrous Na₂SO₄. After filtration, the filtrate was concentrated under reduced pressure. The residue was purified by reversed-phase flash chromatography with the following conditions (column, C18 silica gel; mobile phase, CH₃CN in water (0.1% NH₃·H₂O), 50% to 90% gradient in 10 min; detector, UV 254 nm) to afford N-(7-fluoro-2-methyl-indazol-5-yl)-2-methoxy-7-[(3S)-3-(methylamino)pyrrolidin-1-yl]-1,3-benzothiazole-4-carboxamide (21 mg, 46.20 μmol, 37% yield) as a solid. LCMS (ES, m/z): 455 [M+H]⁺. ¹H NMR (400 MHz, DMSO-d₆) δ 11.46 (s, 1H), 8.41 (d, J=2.8 Hz, 1H), 8.06 (d, J=1.6 Hz, 1H), 8.01 (d, J=8.8 Hz, 1H), 7.34 (dd, J=13.2, 1.6 Hz, 1H), 6.56 (d, J=8.9 Hz, 1H), 4.40 (s, 3H), 4.19 (s, 3H), 3.76 (dd, J=9.2, 5.9 Hz, 1H), 3.72-3.62 (m, 1H), 3.61 (d, J=6.3 Hz, 1H), 3.39 (dd, J=9.1, 4.1 Hz, 2H), 3.30 (t, J=5.1 Hz, 1H), 2.32 (s, 3H), 2.10 (dd, J=12.6, 6.0 Hz, 1H), 1.93-1.82 (m, 1H).

### Example 275: Synthesis of Compound 749

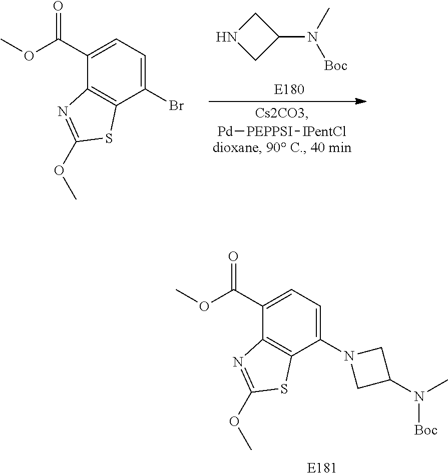

To a stirred mixture of methyl 7-bromo-2-methoxy-1,3-benzothiazole-4-carboxylate (100.0 mg, 330.97 μmol) and tert-butyl N-(azetidin-3-yl)-N-methyl-carbamate (123.3 mg, 661.94 μmol) in dioxane (2 mL) were added Cs₂CO₃ (161.7 mg, 496.45 μmol), Pd-PEPPSI-IPentCl (27.7 mg, 33.10 μmol) at room temperature under nitrogen atmosphere. The resulting mixture was stirred for 1 h at 90° C. under nitrogen atmosphere. The mixture was allowed to cool down to room temperature. The resulting mixture was diluted with water (10 mL). The resulting mixture was extracted with EA (2×5 mL). The combined organic layers were washed with water (2×5 mL), brine (1×5 mL), dried over anhydrous Na₂SO₄. After filtration, the filtrate was concentrated under reduced pressure. The residue was purified by silica gel column chromatography, eluted with PE/EA (2:1) to afford methyl 7-[3-[tert-butoxycarbonyl(methyl)amino]azetidin-1-yl]-2-methoxy-1,3-benzothiazole-4-carboxylate (80 mg, 196.33 μmol, 59.32% yield) as a solid. LCMS (ES, m/z): 408 [M+H]⁺.

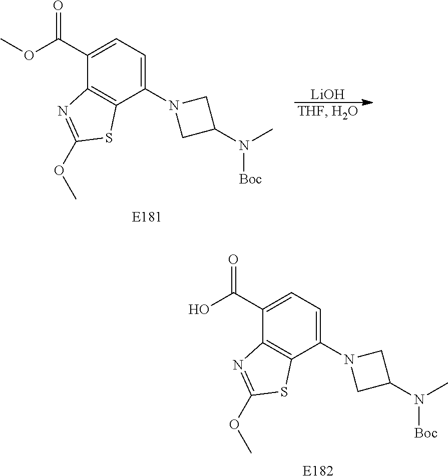

To a stirred solution of methyl 7-[3-[tert-butoxycarbonyl(methyl)amino]azetidin-1-yl]-2-methoxy-1,3-benzothiazole-4-carboxylate (75.0 mg, 184.06 μmol) in MeOH (0.5 mL), THF (0.5 mL) was added LiOH (4.4 mg, 184.06 μmol) in portions at rt. The resulting mixture was stirred for 4 h at 50° C. The reaction was monitored by LCMS. The resulting mixture was diluted with water (20 mL). The mixture was acidified to pH 6 with HCl (2N). The resulting mixture was extracted with EA (2×20 mL). The combined organic layers were washed with brine (1×20 mL), dried over anhydrous Na₂SO₄. After filtration, the filtrate was concentrated under reduced pressure. This resulted in 7-[3-[tert-butoxycarbonyl(methyl)amino]azetidin-1-yl]-2-methoxy-1,3-benzothiazole-4-carboxylic acid (50 mg, 127.08 μmol, 69% yield) as a solid. LCMS (ES, m/z): 394 [M+H]⁺.

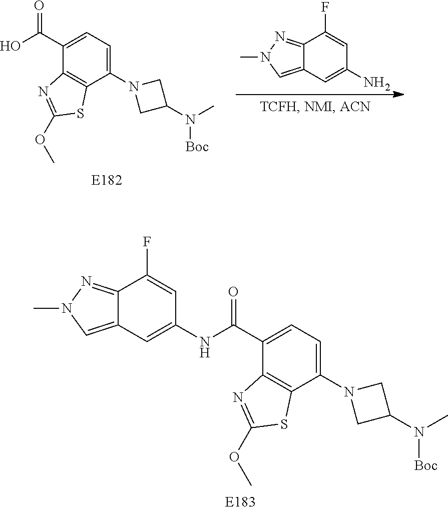

To a stirred mixture of 7-[3-[tert-butoxycarbonyl(methyl)amino]azetidin-1-yl]-2-methoxy-1,3-benzothiazole-4-carboxylic acid (50.0 mg, 127.08 μmol) and 7-fluoro-2-methyl-indazol-5-amine (23.1 mg, 139.79 μmol) in ACN (1 mL) was added NMI (52.1 mg, 635.39 μmol) and TCFH (49.92 mg, 177.91 μmol) at room temperature. The resulting mixture was stirred for 3 h at 25° C. The reaction was monitored by LCMS. The resulting mixture was diluted with water (2 mL). The precipitated solids were collected by filtration and washed with ACN (1 mL). This resulted in tert-butyl N-[1-[4-[(7-fluoro-2-methyl-indazol-5-yl)carbamoyl]-2-methoxy-1,3-benzothiazol-7-yl]azetidin-3-yl]-N-methyl-carbamate (48 mg, 88.79 μmol, 70% yield) as a solid. LCMS (ES, m/z): 541 [M+H]⁺.

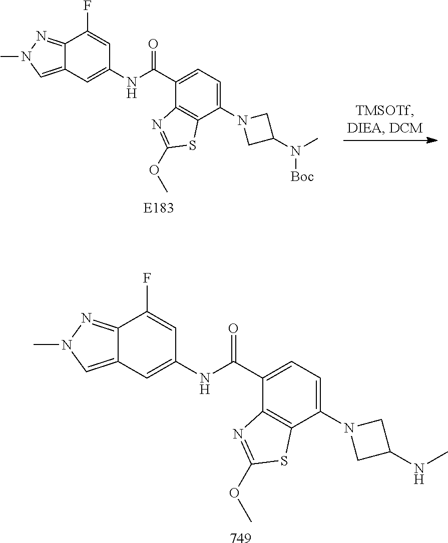

To a solution of tert-butyl N-[1-[4-[(7-fluoro-2-methyl-indazol-5-yl)carbamoyl]-2-methoxy-1,3-benzothiazol-7-yl]azetidin-3-yl]-N-methyl-carbamate (35.0 mg, 64.74 μmol) in DCM (1 mL) was added DIEA (16.7 mg, 129.48 μmol) and TMSOTf (43.1 mg, 194.23 μmol). The reaction was stirred for 1 h at rt. The resulting mixture was concentrated under reduced pressure and adjusted to pH 8 with saturated NaHCO₃ (aq.). The resulting mixture was diluted with water (5 mL) and extracted with DCM/MeOH (5/1) (2×10 mL). The combined organic layers were washed with water (1×5 mL), brine (1×5 mL) and dried over anhydrous Na₂SO₄. After filtration, the filtrate was concentrated under reduced pressure. The residue was purified by reversed-phase flash chromatography with the following conditions (column, C18 silica gel; mobile phase, CH₃CN in water (0.1% NH₃·H₂O), 50% to 90% gradient in 10 min; detector, UV 254 nm) to afford N-(7-fluoro-2-methyl-indazol-5-yl)-2-methoxy-7-[3-(methylamino) azetidin-1-yl]-1,3-benzothiazole-4-carboxamide (8 mg, 18.16 μmol, 28% yield) as a solid. LCMS (ES, m/z): 441 [M+H]⁺. ¹H NMR (400 MHz, DMSO-d₆) δ 11.38 (s, 1H), 8.42 (d, J=2.8 Hz, 1H), 8.09-8.00 (m, 2H), 7.34 (d, J=12.9 Hz, 1H), 6.45 (d, J=8.6 Hz, 1H), 4.41 (s, 3H), 4.30 (t, J=7.2 Hz, 2H), 4.19 (s, 3H), 3.86-3.78 (m, 2H), 3.66-3.57 (m, 1H), 2.50 (s, 1H), 2.34 (s, 1H), 2.26 (s, 3H).

### Example 276: Synthesis of Compound 750

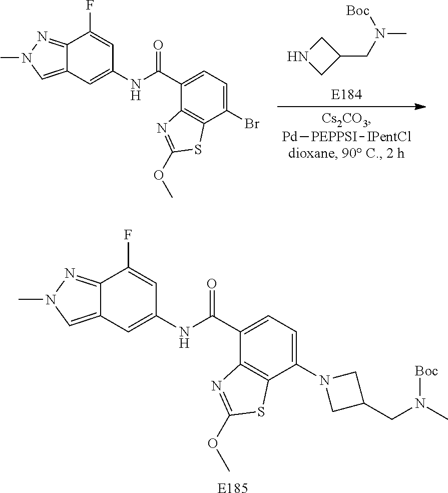

To a stirred mixture of 7-bromo-N-(7-fluoro-2-methyl-indazol-5-yl)-2-methoxy-1,3-benzothiazole-4-carboxamide (0.11 g, 252.72 μmol) and tert-butyl N-(azetidin-3-ylmethyl)-N-methyl-carbamate (60.7 mg, 303.26 μmol) in Dioxane (2 mL) were added Cs₂CO₃ (164.8 mg, 505.43 μmol) Pd-PEPPSI™-IPent catalyst (20.1 mg, 25.27 μmol) at room temperature under nitrogen atmosphere. The resulting mixture was stirred for 2 h at 90° C. under nitrogen atmosphere. The mixture was allowed to cool down to room temperature. The mixture was concentrated under reduced pressure. The mixture was purified by silica gel column chromatography, eluted with PE/EA (1:8) to afford tert-butyl N-[[1-[4-[(7-fluoro-2-methyl-indazol-5-yl)carbamoyl]-2-methoxy-1,3-benzothiazol-7-yl]azetidin-3-yl]methyl]-N-methyl-carbamate (0.04 g, 72.12 μmol, 29% yield) as a solid. LCMS (ES, m/z): 555 [M+H]⁺.

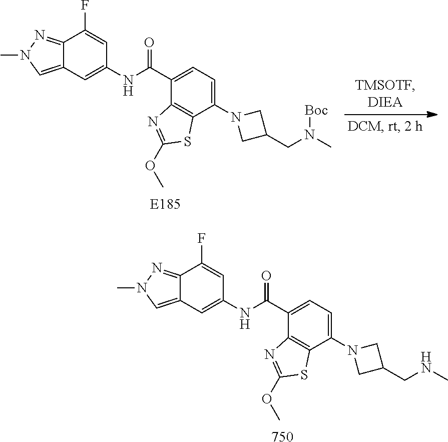

To a solution of tert-butyl N-[[1-[4-[(7-fluoro-2-methyl-indazol-5-yl)carbamoyl]-2-methoxy-1,3-benzothiazol-7-yl]azetidin-3-yl]methyl]-N-methyl-carbamate (0.038 g, 68.51 μmol) in DCM (1 mL) was added DIEA (26.6 mg, 205.54 μmol) TMSOTF (30.6 mg, 137.03 μmol). The reaction was stirred for 2 h at rt. After filtration, the filtrate was concentrated under reduced pressure. The residue was purified by reversed-phase flash chromatography with the following conditions (SunFire Prep C18 OBD Column 19*150 mm, 5 μm 10 nm, mobile phase, MeCN in water (0.05% TFA), 10% to 30% gradient in 7 min; detector, UV 254 nm) to afford N-(7-fluoro-2-methyl-2H-indazol-5-yl)-2-methoxy-7-(3-((methylamino)methyl) azetidin-1-yl)benzo[d]thiazole-4-carboxamide 2,2,2-trifluoroacetate (0.01 g, 17.59 μmol, 26% yield) as a solid. LCMS (ES, m/z): 455 [M+H]⁺. ¹H NMR (400 MHz, DMSO-d₆) δ 11.37 (s, 1H), 8.49 (s, 2H), 8.42 (d, J=2.8 Hz, 1H), 8.07 (dd, J=5.1, 3.6 Hz, 2H), 7.34 (d, J=13.1 Hz, 1H), 6.47 (d, J=8.6 Hz, 1H), 4.42 (s, 3H), 4.28 (t, J=7.8 Hz, 2H), 4.19 (s, 3H), 4.02-3.94 (m, 2H), 3.30 (d, J=7.2 Hz, 2H), 3.10-3.02 (m, 1H), 2.61 (s, 3H).

### Example 277: Synthesis of Compound 752

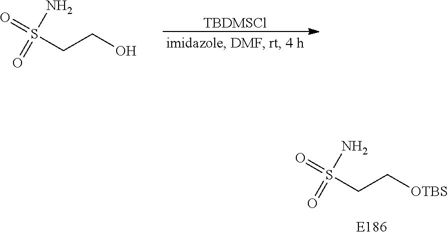 [SMILES: NS(=O)(=O)CCO ; "TBDMSCl" ; CC(C)(C)[Si](C)(C)OCCS(N)(=O)=O]

To a stirred mixture of 2-hydroxyethanesulfonamide (500 mg, 4.00 mmol) in DMF (10 mL) was added imidazole (326.40 mg, 4.79 mmol) and TBDMSCl (722.6 mg, 4.79 mmol) at room temperature. The resulting mixture was stirred for 4 h at rt. The reaction was quenched with water (50 mL) at 0° C. The resulting mixture was extracted with EA (2×20 mL). The combined organic layers were washed with brine (2×50 mL), dried over anhydrous Na₂SO₄. After filtration, the filtrate was concentrated under reduced pressure. The residue was purified by trituration with PE (10 mL). The precipitated solids were collected by filtration and washed with PE (2×5 mL). This resulted in 2-[tert-butyl(dimethyl)silyl]oxyethanesulfonamide (700 mg, 2.92 mmol, 73% yield) as a solid. ¹H NMR (400 MHz, Chloroform-d) δ 4.88 (s, 2H), 4.13 (t, J=5.5 Hz, 2H), 3.33 (t, J=5.6 Hz, 2H), 0.93 (d, J=1.2 Hz, 9H), 0.14 (d, J=1.2 Hz, 6H).

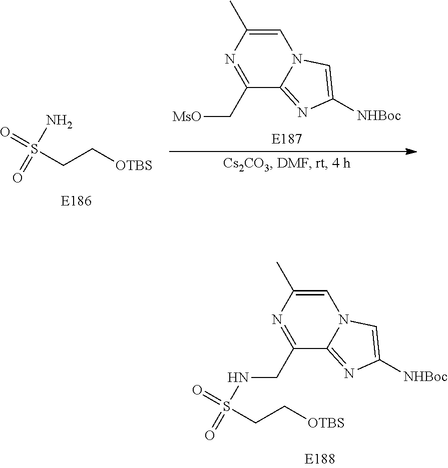 [SMILES: Cc1cn2cc(NC(=O)OC(C)(C)C)nc2c(COS(C)(=O)=O)n1 ; CC(C)(C)[Si](C)(C)OCCS(N)(=O)=O ; Cc1cn2cc(NC(=O)OC(C)(C)C)nc2c(CNS(=O)(=O)CCO[Si](C)(C)C(C)(C)C)n1]

To a stirred mixture of [2-(tert-butoxycarbonylamino)-6-methyl-imidazo[1,2-a]pyrazin-8-yl]methyl methanesulfonate (310 mg, 869.82 μmol) and 2-[tert-butyl(dimethyl)silyl]oxyethanesulfonamide (208.2 mg, 869.82 μmol) in DMF (4 mL) was added Cs₂CO₃ (226.7 mg, 695.85 μmol) at room temperature. The resulting mixture was stirred for 4 h at room temperature. The resulting mixture was diluted with water (10 mL). The resulting mixture was extracted with EA (3×10 mL). The combined organic layers were washed with water (3×30 mL) and brine (1×30 mL), dried over anhydrous Na₂SO₄. After filtration, the filtrate was concentrated under reduced pressure. The residue was purified by silica gel column chromatography, eluted with PE/EA (2:1) to afford tort-butyl N-[8-[[2-[tert-butyl(dimethyl)silyl]oxyethylsulfonylamino]methyl]-6-methyl-imidazo[1,2-a]pyrazin-2-yl]carbamate (65 mg, 130.08 μmol, 14.95% yield) as a solid. LCMS (ES, m/z): 500 [M+H]⁺.

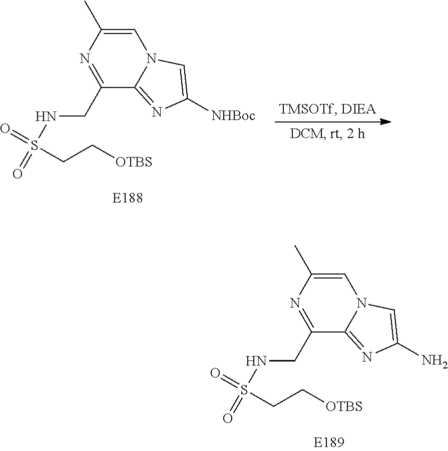 [SMILES: Cc1cn2cc(NC(=O)OC(C)(C)C)nc2c(CNS(=O)(=O)CCO[Si](C)(C)C(C)(C)C)n1 ; Cc1cn2cc(N)nc2c(CNS(=O)(=O)CCO[Si](C)(C)C(C)(C)C)n1]

To a stirred mixture of tort-butyl N-[8-[[2-[tert-butyl(dimethyl)silyl]oxyethylsulfonylamino]methyl]-6-methyl-imidazo[1,2-a]pyrazin-2-yl]carbamate (65 mg, 130.08 μmol) in DCM (1.20 mL) were added DIEA (67.3 mg, 520.31 μmol) and TMSOTf (86.7 mg, 390.24 μmol) dropwise at 0° C. The resulting mixture was stirred for 2 h at room temperature. The resulting mixture were concentrated under reduced pressure and diluted with water (2 mL). The mixture was basified to pH 8 with saturated NaHCO₃ (aq.) and extracted with DCM (3×2 mL). The combined organic layers were washed with water (2×5 mL) and brine (1×5 mL), dried over anhydrous Na₂SO₄. After filtration, the filtrate was concentrated under reduced pressure. The residue was purified by silica gel column chromatography, eluted with EA to afford N-[(2-amino-6-methyl-imidazo[1,2-a]pyrazin-8-yl)methyl]-2-[tert-butyl(dimethyl)silyl]oxy-ethanesulfonamide (50 mg, 125.13 μmol, 96.20% yield) as a solid. LCMS (ES, m/z): 400 [M+H]⁺.

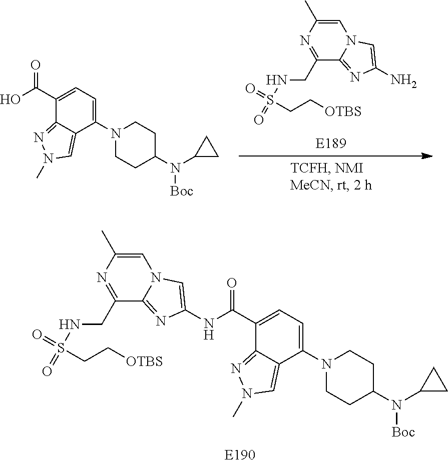 [SMILES: Cn1cc2c(N3CCC(N(C(=O)OC(C)(C)C)C4CC4)CC3)ccc(C(=O)O)c2n1 ; Cc1cn2cc(N)nc2c(CNS(=O)(=O)CCO[Si](C)(C)C(C)(C)C)n1 ; Cc1cn2cc(NC(=O)c3ccc(N4CCC(N(C(=O)OC(C)(C)C)C5CC5)CC4)c4cn(C)nc34)nc2c(CNS(=O)(=O)CCO[Si](C)(C)C(C)(C)C)n1]

To a stirred mixture of 4-[4-[tert-butoxycarbonyl(cyclopropyl)amino]-1-piperidyl]-2-methyl-indazole-7 carboxylic acid (70 mg, 168.88 μmol) and N-[(2-amino-6-methyl-imidazo[1,2-a]pyrazin-8-yl)methyl]-2-[[tert-butyl(dimethyl)silyl]oxy-ethanesulfonamide (67.5 mg, 168.88 μmol) in MeCN (1.5 mL) were added TCFH (71.1 mg, 253.32 μmol) and NMI (41.6 mg, 506.64 μmol) at room temperature. The resulting mixture was stirred for 2 h at room temperature. The resulting mixture was diluted with water (5 mL). The precipitated solids were collected by filtration and washed with water (3×10 mL) to afford tert-butyl N-[1-[7-[8-[2-[[tert-butyl(dimethyl)silyl]oxyethylsulfonylamino]methyl]-6-methyl-imidazo[1,2-a]pyrazin-2-yl]carbamoyl]-2-methyl-indazol-4-yl]-4-piperidyl]-N-cyclopropyl-carbamate (60 mg, 75.37 μmol, 45% yield) as a solid. LCMS (ES, m/z): 796 [M+H]⁺.

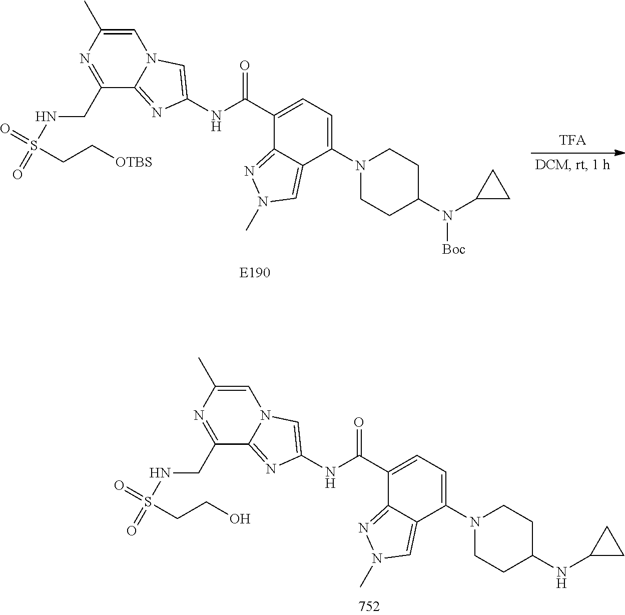 [SMILES: Cc1cn2cc(NC(=O)c3ccc(N4CCC(N(C(=O)OC(C)(C)C)C5CC5)CC4)c4cn(C)nc34)nc2c(CNS(=O)(=O)CCO[Si](C)(C)C(C)(C)C)n1 ; Cc1cn2cc(NC(=O)c3ccc(N4CCC(NC5CC5)CC4)c4cn(C)nc34)nc2c(CNS(=O)(=O)CCO)n1]

To a stirred mixture of tert-butyl N-[1-[7-[[8-[[2-[tert-butyl(dimethyl)silyl]oxyethylsulfonylamino]methyl]-6-methyl-imidazo[1,2-a]pyrazin-2-yl]carbamoyl]-2-methyl-indazol-4-yl]-4-piperidyl]-N-cyclopropyl-carbamate (30 mg, 37.69 μmol) in DCM (1 mL) was added TFA (0.3 mL) dropwise at room temperature. The resulting mixture was stirred for 2 h at room temperature. The resulting mixture were concentrated under reduced pressure. The crude product was purified by Prep-HPLC with the following conditions (column, C18 silica gel, XBridge, 19×150 mm; mobile phase, MeCN in water (0.05% NH₃H₂O), 30% to 80% gradient in 7 min; detector, UV 254 nm) to afford 4-[4-(cyclopropylamino)-1-piperidyl]-N-[8-[(2-hydroxyethylsulfonylamino)methyl]-6-methyl-imidazo[1,2-a]pyrazin-2-yl]-2-methyl-indazole-7-carboxamide (4.6 mg, 7.91 μmol, 21% yield) as a solid. LCMS (ES, m/z): 582 [M+H]⁺. ¹H NMR (400 MHz, DMSO-d₆) δ 11.50 (s, 1H), 8.80 (s, 1H), 8.42 (d, J=11.3 Hz, 2H), 8.03 (d, J=8.1 Hz, 1H), 7.46 (s, 1H), 6.51 (d, J=8.3 Hz, 1H), 4.93 (t, J=5.7 Hz, 1H), 4.67 (d, J=3.6 Hz, 2H), 4.31 (s, 3H), 3.90 (d, J=13.1 Hz, 2H), 3.81 (q, J=6.3 Hz, 2H), 3.35 (t, J=6.6 Hz, 2H), 3.09 (t, J=11.8 Hz, 2H), 2.79 (d, J=4.0 Hz, 1H), 2.44 (s, 3H), 2.13 (s, 1H), 2.01 (d, J=12.7 Hz, 2H), 1.48 (q, J=11.0 Hz, 2H), 0.40 (dt, J=6.1, 2.9 Hz, 2H), 0.24 (dd, J=6.4, 3.5 Hz, 2H).

### Example 278: Synthesis of Compound 753

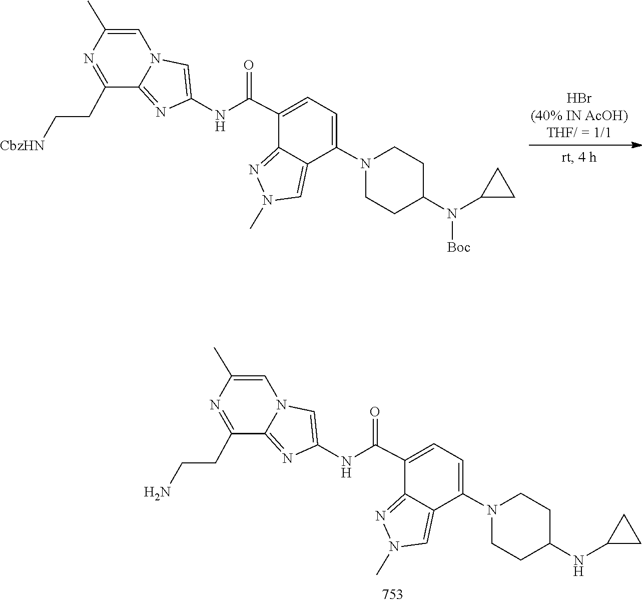 [SMILES: Cc1cn2cc(NC(=O)c3ccc(N4CCC(N(C(=O)OC(C)(C)C)C5CC5)CC4)c4cn(C)nc34)nc2c(CCNC(=O)OCc2ccccc2)n1 ; Cc1cn2cc(NC(=O)c3ccc(N4CCC(NC5CC5)CC4)c4cn(C)nc34)nc2c(CCN)n1]

To a stirred mixture of tert-butyl N-[1-[7-[[8-[2-(benzyloxycarbonylamino)ethyl]-6-methyl-imidazo[1,2-a]pyrazin-2-yl]carbamoyl]-2-methyl-indazol-4-yl]-4-piperidyl]-N-cyclopropyl-carbamate (30 mg, 41.56 μmol) in THF (1 mL) was added HBr (40% in AcOH) (1 mL) dropwise at room temperature. The resulting mixture was stirred for 2 h at room temperature. The resulting mixture were concentrated under reduced pressure and diluted with water (2 mL). The mixture was basified to pH 8 with saturated NaHCO₃ (aq.) and extracted with DCM (2×2 mL). The combined organic layers were washed with water (2×10 mL) and brine (1×10 mL), dried over anhydrous Na₂SO₄. After filtration, the filtrate was concentrated under reduced pressure. The crude product was purified by Prep-HPLC with the following conditions (column, C18 silica gel, XBridge, 19×150 mm; mobile phase, MeCN in water (0.05% NH₃H₂O), 20% to 50% gradient in 7 min; detector, UV 254 nm) to afford N-[8-(2-aminoethyl)-6-methyl-imidazo[1,2-a]pyrazin-2-yl]-4-[4-(cyclopropylamino)-1-piperidyl]-2-methyl-indazole-7-carboxamide (4 mg, 8.20 μmol, 20% yield) as a solid. LCMS (ES, m/z): 488 [M+H]⁺. ¹H NMR (400 MHz, DMSO-d₆) δ 11.48 (s, 1H), 8.89 (s, 1H), 8.84 (s, 2H), 8.41 (d, J=7.0 Hz, 2H), 8.07 (d, J=8.1 Hz, 1H), 7.87 (s, 3H), 6.58 (d, J=8.2 Hz, 1H), 4.31 (s, 3H), 4.06 (d, J=13.0 Hz, 2H), 3.46-3.37 (m, 4H), 3.07 (t, J=12.4 Hz, 2H), 2.80 (s, 1H), 2.43 (s, 3H), 2.22 (d, J=12.3 Hz, 2H), 2.08 (s, 4H), 1.83-1.70 (m, 2H), 0.85 (d, J=5.5 Hz, 4H).

### Example 279: Synthesis of Compound 754

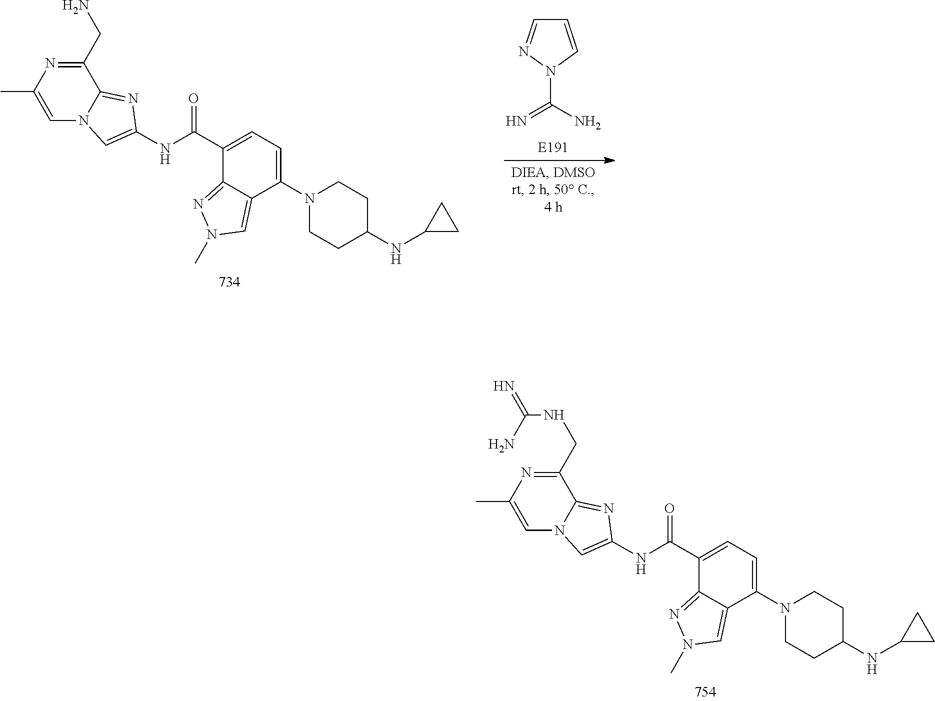 [SMILES: Cc1cn2cc(NC(=O)c3ccc(N4CCC(NC5CC5)CC4)c4cn(C)nc34)nc2c(CN)n1 ; N=C(N)n1cccn1 ; Cc1cn2cc(NC(=O)c3ccc(N4CCC(NC5CC5)CC4)c4cn(C)nc34)nc2c(CNC(=N)N)n1]

To a stirred mixture of N-[8-(aminomethyl)-6-methyl-imidazo[1,2-a]pyrazin-2-yl]-4-[4-(cyclopropylamino)-1-piperidyl]-2-methyl-indazole-7-carboxamide (44 mg, 92.91 μmol) and 1H-pyrazole-1-carboximidamide hydrochloride (20.4 mg, 139.37 μmol) in DMSO (1.0 mL) was added DIEA (36.0 mg, 278.73 μmol) at room temperature. The crude product was purified by Prep-HPLC with the following conditions (column, C18 silica gel, XBridge, 19×150 mm; mobile phase, MeCN in water (0.05% TFA), 25% to 55% gradient in 7 min; detector, UV 254 nm) to afford 4-(4-(cyclopropylamino)piperidin-1-yl)-N-(8-(guanidinomethyl)-6-methylimidazo[1,2-a]pyrazin-2-yl)-2-methyl-2H-indazole-7-carboxamide bis(2,2,2-trifluoroacetate) (1.1 mg, 1.48 μmol, 2% yield) as a solid. LCMS (ES, m/z): 516 [M+H]⁻. ¹H NMR (300 MHz, DMSO-d₆) δ 11.47 (s, 1H), 8.87 (s, 1H), 8.73 (s, 2H), 8.45 (d, J=11.5 Hz, 2H), 8.05 (d, J=8.0 Hz, 1H), 7.82 (s, 1H), 7.40-7.16 (d, J=50.9 Hz, 3H), 6.56 (d, J=8.3 Hz, 1H), 4.88 (d, J=5.3 Hz, 2H), 4.28 (s, 3H), 4.05 (d, J=13.3 Hz, 2H), 3.05 (t, J=12.5 Hz, 2H), 2.78 (s, 1H), 2.71 (s, 1H), 2.44 (s, 3H), 2.19 (d, J=12.2 Hz, 2H), 1.73 (d, J=9.0 Hz, 2H), 0.83 (d, J=7.3 Hz, 4H).

### Example 280. Synthesis of Compound 836

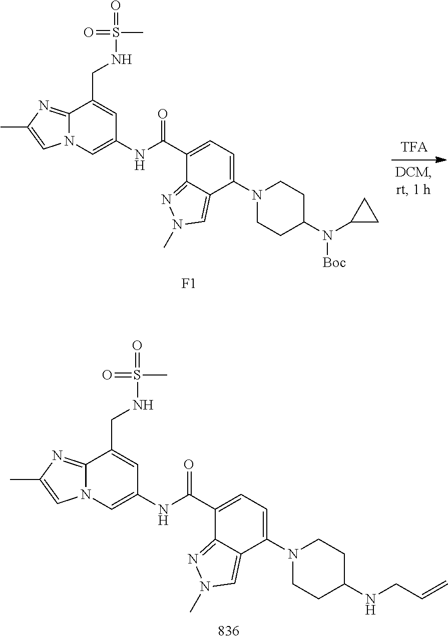 [SMILES: Cc1cn2cc(NC(=O)c3ccc(N4CCC(N(C(=O)OC(C)(C)C)C5CC5)CC4)c4cn(C)nc34)cc(CNS(C)(=O)=O)c2n1 ; "F1" ; C=CCNC1CCN(c2ccc(C(=O)Nc3cc(CNS(C)(=O)=O)c4nc(C)cn4c3)c3nn(C)cc23)CC1]

To a stirred mixture of tert-butyl N-cyclopropyl-N-[1-[7-[[8-(methanesulfonamidomethyl)-2-methyl-imidazo[1,2-a]pyridin-6-yl]carbamoyl]-2-methyl-indazol-4-yl]-4-piperidyl]carbamate (Intermediate F1, 33 mg, 50.71 μmol) in DCM (2 mL) was added TFA (1 mL) dropwise at room temperature. The resulting mixture was stirred for 1 h at room temperature. The resulting mixture was concentrated under reduced pressure. The crude product was purified by Prep-HPLC with the following conditions (column, C18 silica gel, XBridge, 19×150 mm; mobile phase, MeCN in water (0.05% NH₃·H₂O), 35% to 55% gradient in 7 min; detector, UV 254 nm) to afford 4-[4-(allylamino)-1-piperidyl]-N-[8-(methanesulfonamidomethyl)-2-methyl-imidazo[1,2-a]pyridin-6-yl]-2-methyl-indazole-7-carboxamide (Compound 836, 2.3 mg, 4.18 μmol). LCMS (ES, m/z): 551 [M+H]0.1H NMR (400 MHz, Methanol-d₄) δ 9.06 (d, J=1.9 Hz, 1H), 8.34 (s, 1H), 7.95 (d, J=8.0 Hz, 1H), 7.52 (s, 1H), 7.19 (t, J=1.5 Hz, 1H), 6.40 (d, J=8.1 Hz, 1H), 5.95 (ddt, J=16.8, 10.2, 6.4 Hz, 1H), 5.35 (dd, J=17.2, 1.7 Hz, 1H), 5.27 (d, J=10.2 Hz, 1H), 4.54 (s, 2H), 4.24 (s, 3H), 3.91 (d, J=12.7 Hz, 2H), 3.44 (d, J=6.4 Hz, 2H), 3.07 (s, 3H), 2.93 (t, J=11.6 Hz, 3H), 2.38 (s, 3H), 2.15-2.02 (m, 2H), 1.62 (qd, J=12.2, 4.0 Hz, 2H).

### Example 281. Synthesis of Compound 837

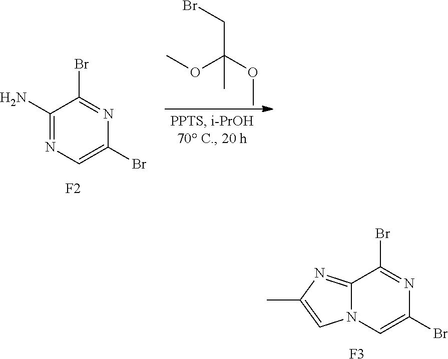

To a solution of 3,5-dibromopyrazin-2-amine (Intermediate F1, 10 g, 39.54 mmol) in i-PrOH (150 mL) were added 1-bromo-2,2-dimethoxy-propane (21.71 g, 118.63 mmol) and PPTS (1.99 g, 7.91 mmol) at rt. The reaction was stirred for 20 h at 70° C. The reaction mixture was cooled to room temperature and concentrated under reduced pressure. The residue was purified by silica gel column chromatography, eluted with PE/THF (3/1) to afford 6,8-dibromo-2-methyl-imidazo[1,2-a]pyrazine (Intermediate F3, 5 g, 17.19 mmol). LCMS (ES, m/z): 290 [M+H]⁺.

### Synthesis of Intermediate F4

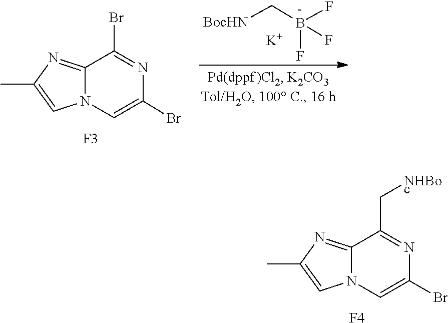

To a stirred mixture of 6,8-dibromo-2-methyl-imidazo[1,2-a]pyrazine (Intermediate F3, 4.85 g, 16.67 mmol) in toluene (50 mL)/H₂O (5 mL) were added potassium (((tert-butoxycarbonyl)amino)methyl)trifluoroborate (4.35 g, 18.34 mmol), K₂CO₃ (6.91 g, 50.01 mmol) and Pd(dppf)Cl₂·CH₂Cl₂ (1.36 g, 1.67 mmol) at rt under N₂. The mixture was stirred for 16 h at 100° C. The reaction was diluted with H₂O (100 mL). The resulting mixture was extracted with EA (2×100 mL). The combined organic layers were washed with H₂O (100 mL), dried over anhydrous Na₂SO₄. After filtration, the filtrate was concentrated under reduced pressure. The residue was purified by Prep-HPLC with the following conditions (MeCN in water (0.05% FA), 0% to 40% gradient in 8 min; detector, UV 254 nm) to afford tert-butyl N-[(6-bromo-2-methyl-imidazo[1,2-a]pyrazin-8-yl)methyl]carbamate (Intermediate F4, 1.4 g, 4.10 mmol). LCMS (ES, m/z): 341 [M+H]⁺.

### Synthesis of Intermediate F6

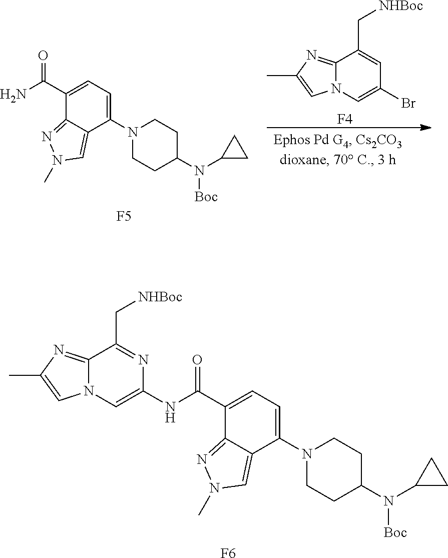

To a stirred mixture of tert-butyl N-[1-(7-carbamoyl-2-methyl-indazol-4-yl)-4-piperidyl]-N-cyclopropyl-carbamate (Intermediate F5, 250 mg, 604.58 μmol) in dioxane (3 mL) were added tert-butyl N-[(6-bromo-2-methyl-imidazo[1,2-a]pyrazin-8-yl)methyl]carbamate (Intermediate F4, 226.9 mg, 665.04 μmol), Cs₂CO₃ (393.9 mg, 1.21 mmol) and EPhos Pd G4 (55.5 mg, 60.46 μmol) at rt under N₂. The mixture was stirred for 3 h at 70° C. under N₂. The reaction was diluted with H₂O (15 mL). The resulting mixture was extracted with DCM (2×15 mL). The combined organic layers were washed with H₂O (15 mL), dried over anhydrous Na₂SO₄. After filtration, the filtrate was concentrated under reduced pressure. The residue was purified by silica gel column chromatography, eluted with EA to afford tert-butyl N-[1-[7-[[8-[(tert-butoxycarbonylamino)methyl]-2-methyl-imidazo[1,2-a]pyrazin-6-yl]carbamoyl]-2-methyl-indazol-4-yl]-4-piperidyl]-N-cyclopropyl-carbamate (Intermediate F6, 270 mg, 400.71 μmol). LCMS (ES, m/z): 674 [M+H]⁺.

### Synthesis of Intermediate F7

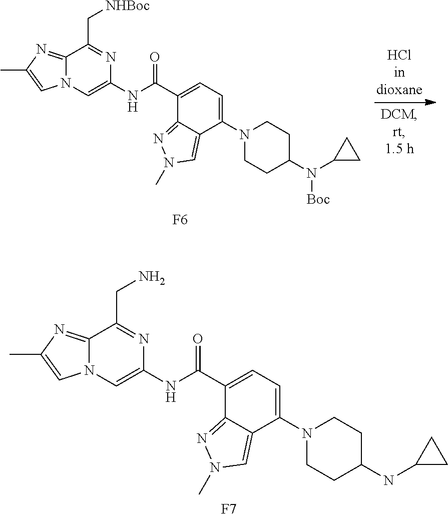

To a solution of tert-butyl N-[1-[7-[[8-[(tert-butoxycarbonylamino)methyl]-2-methyl-imidazo[1,2-a]pyrazin-6-yl]carbamoyl]-2-methyl-indazol-4-yl]-4-piperidyl]-N-cyclopropyl-carbamate (Intermediate F6, 150 mg, 222.62 μmol) in DCM (2 mL) was added HCl (4.0 M in 1,4-dioxane) (556.5 μL). The mixture was stirred for 1.5 h at rt. The resulting mixture was concentrated under reduced pressure to afford N-(8-(aminomethyl)-2-methylimidazo[1,2-a]pyrazin-6-yl)-4-(4-(cyclopropylamino)piperidin-1-yl)-2-methyl-2H-indazole-7-carboxamide hydrochloride (Intermediate F7, 120 mg, crude) as a yellow solid. The crude product was used for next step without further purification. LCMS (ES, m/z): 474 [M+H]⁺.

### Synthesis of Compound 837

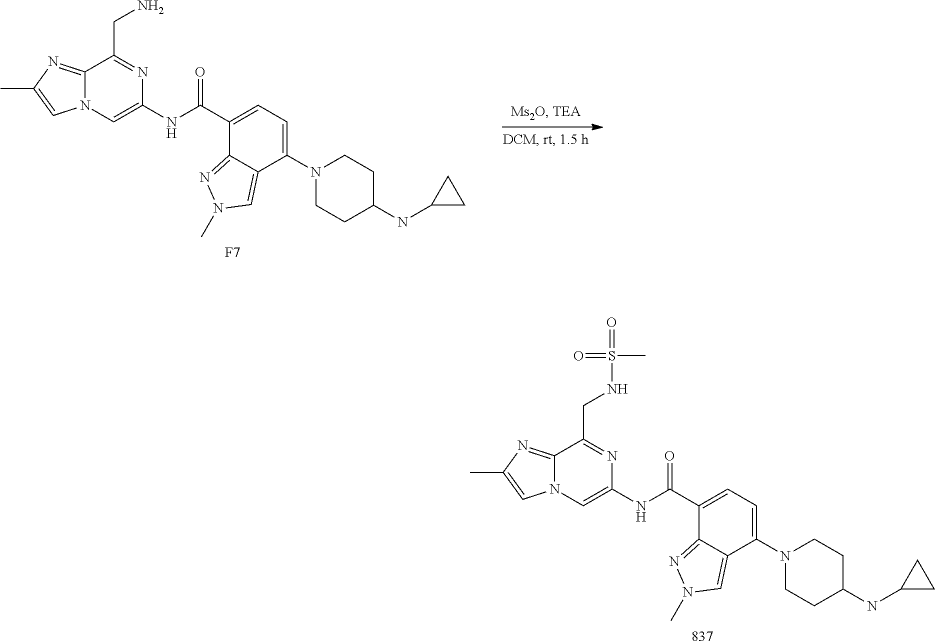

To a solution of N-(8-(aminomethyl)-2-methylimidazo[1,2-a]pyrazin-6-yl)-4-(4-(cyclopropylamino)piperidin-1-yl)-2-methyl-2H-indazole-7-carboxamide hydrochloride (Intermediate F7, 120 mg, 235.28 μmol) in DCM (1.5 mL) were added TEA (47.6 mg, 470.56 μmol) and Ms₂O (45.1 mg, 258.81 μmol). The mixture was stirred for 1.5 h at rt. The reaction was diluted with H₂O (10 mL). The resulting mixture was extracted with DCM (2×10 mL). The combined organic layers were washed with H₂O (10 mL), dried over anhydrous Na₂SO₄. After filtration, the filtrate was concentrated under reduced pressure. The crude product was purified by Prep-HPLC with the following conditions (column, C18 silica gel, XBridge, 19×150 mm; mobile phase, MeCN in water (0.05% NH₃H₂O), 40% to 55% gradient in 7 min; detector, UV 254 nm) to afford 4-[4-(cyclopropylamino)-1-piperidyl]-N-[8-(methanesulfonamidomethyl)-2-methyl-imidazo[1,2-a]pyrazin-6-yl]-2-methyl-indazole-7-carboxamide (Compound 837, 35 mg, 63.44 μmol). LCMS (ES, m/z): 552 [M+H]⁺. ¹H NMR (300 MHz, DMSO-d₆) δ 11.40 (s, 1H), 9.37 (s, 1H), 8.80 (s, 1H), 8.09-7.98 (m, 2H), 7.66 (t, J=5.9 Hz, 1H), 6.50 (d, J=8.3 Hz, 1H), 4.72 (d, J=5.6 Hz, 2H), 4.26 (s, 3H), 3.90 (d, J=12.8 Hz, 2H), 3.21 (s, 3H), 3.08 (t, J=11.8 Hz, 2H), 2.87-2.67 (m, 1H), 2.41 (s, 3H), 2.12 (tt, J=6.7, 3.6 Hz, 1H), 2.01 (d, J=12.4 Hz, 2H), 1.48 (q, J=10.2 Hz, 2H), 0.40 (dt, J=6.2, 3.0 Hz, 2H), 0.27-0.19 (m, 2H).

### Example 282. Synthesis of Compound 846

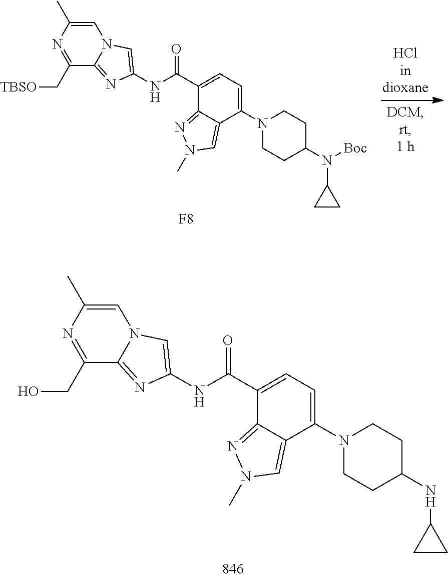 [SMILES: Cc1cn2cc(NC(=O)c3ccc(N4CCC(N(C(=O)OC(C)(C)C)C5CC5)CC4)c4cn(C)nc34)nc2c(CO[Si](C)(C)C(C)(C)C)n1 ; "F8" ; Cc1cn2cc(NC(=O)c3ccc(N4CCC(NC5CC5)CC4)c4cn(C)nc34)nc2c(CO)n1 ; "846"]

To a solution of tert-butyl N-[1-[7-[[8-[[tert-butyl(dimethyl)silyl]oxymethyl]-6-methyl-imidazo[1,2-a]pyrazin-2-yl]carbamoyl]-2-methyl-indazol-4-yl]-4-piperidyl]-N-cyclopropyl-carbamate (Intermediate F8, 50.0 mg, 72.58 μmol) in DCM (1 mL) was added HCl (4.0 M in 1,4-dioxane) (0.4 mL). The reaction was stirred for 1 h at rt. The resulting mixture was concentrated under reduced pressure. The residue was purified by reversed-phase flash chromatography with the following conditions (column, C18 silica gel; mobile phase, CH₃CN in water (0.1% NH₃·H₂O), 50% to 80% gradient in 10 min; detector, UV 254 nm) to afford 4-[4-(cyclopropylamino)-1-piperidyl]-N-[8-(hydroxymethyl)-6-methyl-imidazo[1,2-a]pyrazin-2-yl]-2-methyl-indazole-7-carboxamide (Compound 846, 15 mg, 31.61 μmol). LCMS (ES, m/z): 474 [M+H]⁺. ¹H NMR (400 MHz, DMSO-d₆) δ 11.45 (s, 1H), 8.79 (s, 1H), 8.40 (s, 1H), 8.38 (s, 1H), 8.03 (d, J=8.1 Hz, 1H), 6.50 (d, J=8.2 Hz, 1H), 5.26 (t, J=6.1 Hz, 1H), 4.87 (d, J=6.0 Hz, 2H), 4.31 (s, 3H), 3.90 (d, J=12.7 Hz, 2H), 3.08 (t, J=11.8 Hz, 2H), 2.85-2.75 (m, 1H), 2.43 (s, 3H), 2.16-2.09 (m, 1H), 2.01 (d, J=12.6 Hz, 2H), 1.48 (q, J=11.0 Hz, 2H), 0.40 (dd, J=6.7, 4.5 Hz, 2H), 0.24 (q, J=3.4, 2.7 Hz, 2H).

### Example 283. Synthesis of Compound 848

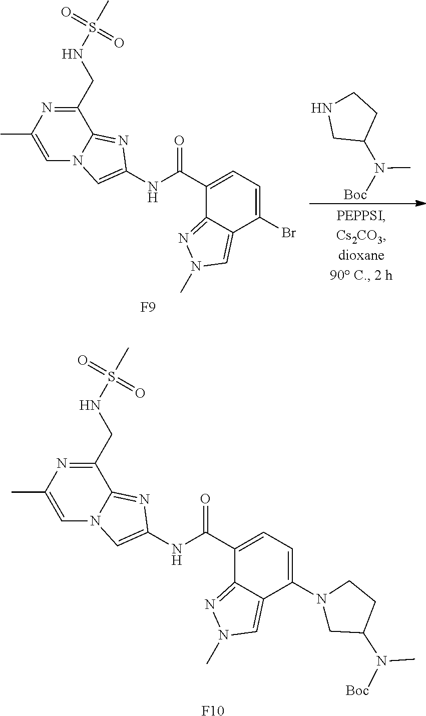

To a stirred mixture of 4-bromo-N-[8-(methanesulfonamidomethyl)-6-methyl-imidazo[1,2-a]pyrazin-2-yl]-2-methyl-indazole-7-carboxamide (Intermediate F9, 70 mg, 142.18 μmol) and tert-butyl N-methyl-N-pyrrolidin-3-yl-carbamate (34.1 mg, 170.61 μmol) in Dioxane (1.5 mL) were added Cs₂CO₃ (92.6 mg, 284.35 μmol) and PEPPSI (11.9 mg, 14.22 μmol) at room temperature under nitrogen atmosphere. The resulting mixture was stirred for 2 h at 90° C. under nitrogen atmosphere. The resulting mixture was concentrated under reduced pressure. The residue was purified by silica gel column chromatography, eluted with PE/EA (1:3) to afford tert-butyl N-[1-[7-[[8-(methanesulfonamidomethyl)-6-methyl-imidazo[1,2-a]pyrazin-2-yl]carbamoyl]-2-methyl-indazol-4-yl]pyrrolidin-3-yl]-N-methyl-carbamate (Intermediate F10, 65 mg, 106.26 μmol). LCMS (ES, m/z): 612 [M+H]⁺.

### Synthesis of Compound 848

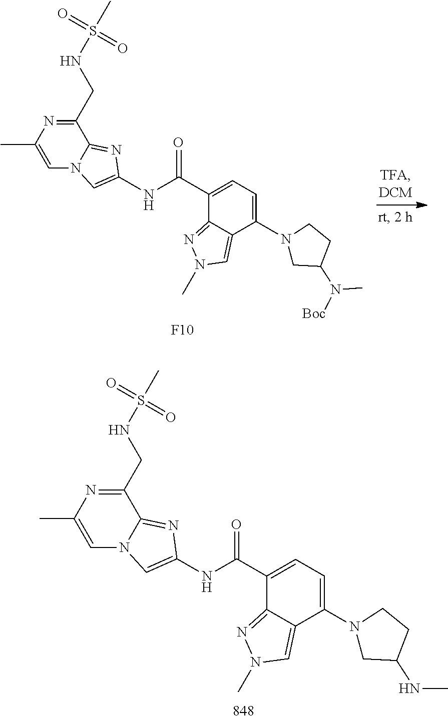

To a stirred solution of tert-butyl N-[1-[7-[[8-(methanesulfonamidomethyl)-6-methyl-imidazo[1,2-a]pyrazin-2-yl]carbamoyl]-2-methyl-indazol-4-yl]pyrrolidin-3-yl]-N-methyl-carbamate (Intermediate F10, 65 mg, 106.26 μmol) in DCM (2 mL) was added TFA (1 mL) at rt. The resulting mixture was stirred for 2 h. The resulting mixture was concentrated under reduced pressure. The crude product was purified by Prep-HPLC with the following conditions (SunFire Prep C18 OBD Column 19*150 mm, 5 Um 10 nm, mobile phase, MeCN in water (0.05% NH₄OH), 40% to 60% gradient in 7 min; detector, UV 254 nm) to afford N-[8-(methanesulfonamidomethyl)-6-methyl-imidazo[1,2-a]pyrazin-2-yl]-2-methyl-4-[3-(methylamino)pyrrolidin-1-yl]indazole-7-carboxamide (Compound 848, 6 mg, 11.73 μmol, 11.04% yield) as a yellow solid. LCMS (ES, m/z): 512 [M+H]⁺. ¹H NMR (400 MHz, DMSO-d₆) δ 11.43 (s, 1H), 8.86 (s, 1H), 8.43 (s, 1H), 8.38 (s, 1H), 7.98 (d, J=8.2 Hz, 1H), 7.49 (s, 1H), 6.04 (d, J=8.4 Hz, 1H), 4.67 (d, J=4.9 Hz, 2H), 4.27 (s, 3H), 3.75 (q, J=7.2 Hz, 2H), 3.66 (s, 1H), 3.45-3.40 (m, 1H), 3.34-3.32 (m, 1H), 3.07 (s, 3H), 2.44 (s, 3H), 2.35 (s, 3H), 2.20-2.11 (m, 1H), 1.97-1.90 (m, 1H).

### Example 284. Synthesis of Compound 861

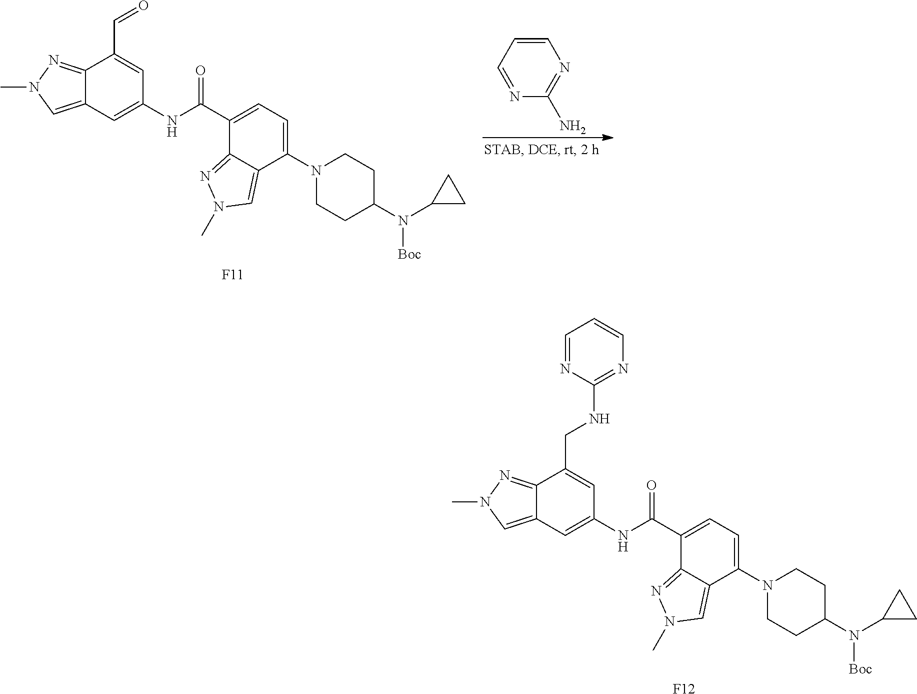

To a solution of tert-butyl N-cyclopropyl-N-[1-[7-[(7-formyl-2-methyl-indazol-5-yl)carbamoyl]-2-methyl-indazol-4-yl]-4-piperidyl]carbamate (Intermediate F11, 120 mg, 209.91 μmol) in DCE (1.5 mL) were added pyrimidin-2-amine (25.9 mg, 272.89 μmol) and STAB (66.7 mg, 314.87 μmol) at rt. The mixture was stirred for 2 h at rt. The reaction was diluted with H₂O (10 mL). The resulting mixture was extracted with DCM (2×10 mL). The combined organic layers were washed with H₂O (2×10 mL), dried over anhydrous Na₂SO₄. After filtration, the filtrate was concentrated under reduced pressure. The residue was purified by silica gel column chromatography, eluted with DCM/MeOH (19/1) to afford tert-butyl N-cyclopropyl-N-[1-[2-methyl-7-[[2-methyl-7-[(pyrimidin-2-ylamino)methyl]indazol-5-yl]carbamoyl]indazol-4-yl]-4-piperidyl]carbamate (Intermediate F12, 85 mg, 50.94 μmol). LCMS (ES, m/z): 651 [M+H]⁺.

### Synthesis of Compound 861

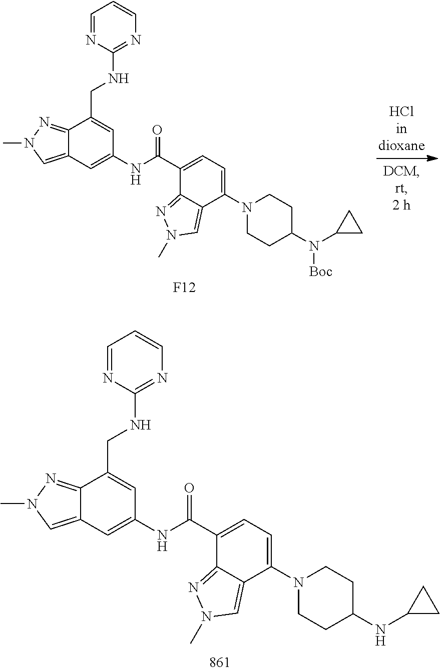

To a solution of tert-butyl N-cyclopropyl-N-[1-[2-methyl-7-[[2-methyl-7-[(pyrimidin-2-ylamino)methyl]indazol-5-yl]carbamoyl]indazol-4-yl]-4-piperidyl]carbamate (Intermediate F12, 85 mg, 50.94 μmol) in DCM (1 mL) was added HCl (4.0 M in 1,4-dioxane) (127 μL). The mixture was stirred for 2 h at rt. The resulting mixture was concentrated under reduced pressure.

The crude product was purified by Prep-HPLC with the following conditions (column, C18 silica gel, XBridge, 19×150 mm; mobile phase, MeCN in water (0.05% NH₃H₂O), 40% to 55% gradient in 7 min; detector, UV 254 nm) to afford 4-[4-(cyclopropylamino)-1-piperidyl]-2-methyl-N-[2-methyl-7-[(pyrimidin-2-ylamino)methyl]indazol-5-yl]indazole-7-carboxamide (Compound 861, 6 mg, 10.90 μmol). LCMS (ES, m/z): 551 [M+H]⁺. ¹H NMR (400 MHz, DMSO-d₆) δ 11.02 (s, 1H), 8.71 (s, 1H), 8.32-8.26 (m, 3H), 8.24 (d, J=1.8 Hz, 1H), 7.92 (d, J=8.1 Hz, 1H), 7.67 (t, J=6.3 Hz, 1H), 7.07 (d, J=1.8 Hz, 1H), 6.59 (t, J=4.8 Hz, 1H), 6.44 (d, J=8.2 Hz, 1H), 4.85 (d, J=6.2 Hz, 2H), 4.16 (d, J=2.9 Hz, 6H), 3.82 (d, J=12.8 Hz, 2H), 3.00 (t, J=11.7 Hz, 2H), 2.73 (d, J=10.1 Hz, 1H), 2.22 (s, 1H), 2.10 (dt, J=6.6, 3.2 Hz, 1H), 1.98 (d, J=12.8 Hz, 2H), 1.45 (d, J=11.3 Hz, 2H), 0.37 (dt, J=6.2, 3.0 Hz, 2H), 0.25-0.17 (m, 2H).

### Example 285. Synthesis of Compound 865

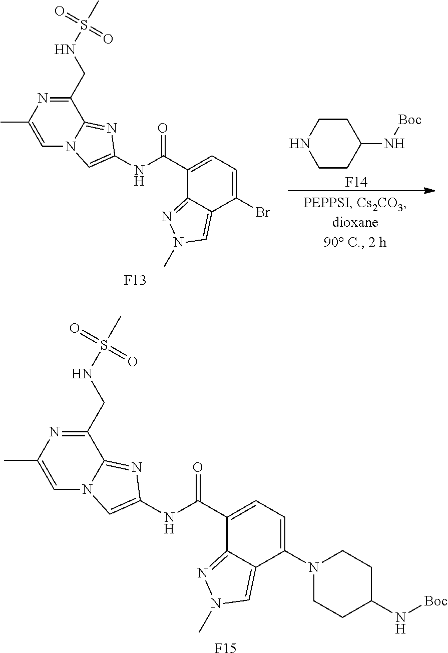

To a stirred mixture of 4-bromo-N-[8-(methanesulfonamidomethyl)-6-methyl-imidazo[1,2-a]pyrazin-2-yl]-2-methyl-indazole-7-carboxamide (Intermediate F13, 50 mg, 101.55 μmol) and tert-butyl N-(4-piperidyl)carbamate (Intermediate F14, 24.4 mg, 121.86 μmol) in Dioxane (1 mL) were added Cs₂CO₃ (66.1 mg, 203.11 μmol) and PEPPSI (8.5 mg, 10.16 μmol) at room temperature under nitrogen atmosphere. The resulting mixture was stirred for 2 h at 90° C. under nitrogen atmosphere. The resulting mixture was concentrated under reduced pressure. The residue was purified by silica gel column chromatography, eluted with PE/EA (1:3) to afford tert-butyl N-[1-[7-[[8-(methanesulfonamidomethyl)-6-methyl-imidazo[1,2-a]pyrazin-2-yl]carbamoyl]-2-methyl-indazol-4-yl]-4-piperidyl]carbamate (Intermediate F15, 40 mg, 65.39 μmol). LCMS (ES, m/z): 612 [M+H]⁺.

### Synthesis of Compound 865

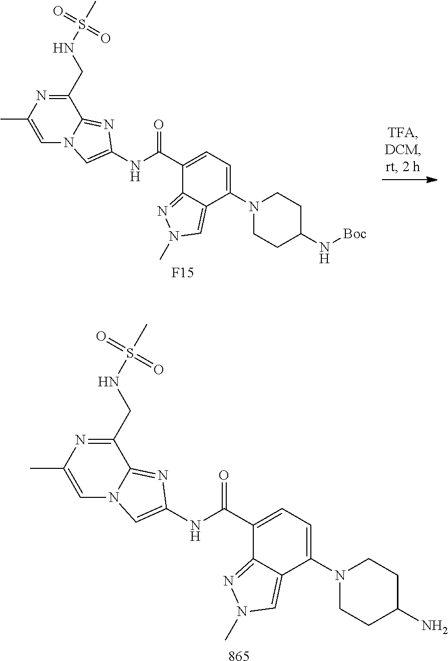

To a stirred solution of tert-butyl N-[1-[7-[[8-(methanesulfonamidomethyl)-6-methyl-imidazo[1,2-a]pyrazin-2-yl]carbamoyl]-2-methyl-indazol-4-yl]-4-piperidyl]carbamate (Intermediate F15, 40 mg, 65.39 μmol) in DCM (2 mL) was added TFA (1 mL) at rt. The resulting mixture was stirred for 2 h. The resulting mixture was concentrated under reduced pressure. The crude product was purified by Prep-HPLC with the following conditions (SunFire Prep C18 OBD Column 19*150 mm, 5 Um 10 nm, mobile phase, MeCN in water (0.05% TFA), 20% to 40% gradient in 7 min; detector, UV 254 nm) to afford 4-(4-amino-1-piperidyl)-N-[8-(methanesulfonamidomethyl)-6-methyl-imidazo[1,2-a]pyrazin-2-yl]-2-methyl-indazole-7-carboxamide (Compound 865, 15 mg, 29.32 μmol). LCMS (ES, m/z): 512 [M+H]⁺. ¹H NMR (400 MHz, DMSO-d₆) δ 11.51 (s, 1H), 8.86 (s, 1H), 8.43 (d, J=14.9 Hz, 2H), 8.06 (d, J=8.1 Hz, 1H), 7.92 (s, 3H), 7.51 (t, J=5.9 Hz, 1H), 6.57 (d, J=8.2 Hz, 1H), 4.67 (d, J=5.9 Hz, 2H), 4.32 (s, 3H), 4.02 (d, J=12.9 Hz, 2H), 3.34 (s, 1H), 3.10 (d, J=12.8 Hz, 2H), 3.07 (s, 3H), 2.45 (s, 3H), 2.05 (d, J=12.6 Hz, 2H), 1.72 (dd, J=11.6 Hz, 2H).

### Example 286. Synthesis of Compound 872

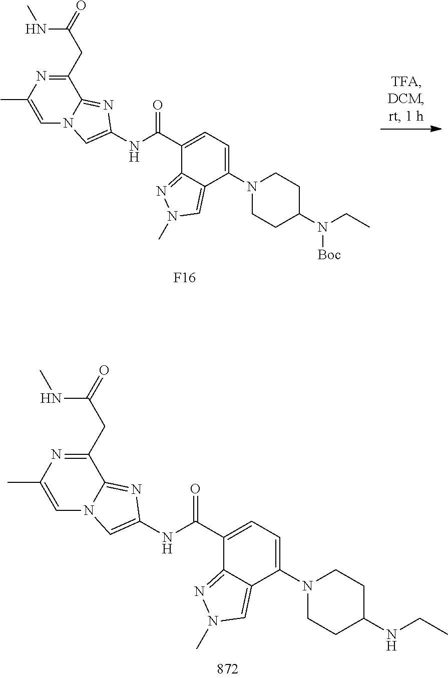

To a stirred mixture of tert-butyl N-ethyl-N-[1-[2-methyl-7-[[6-methyl-8-[2-(methylamino)-2-oxo-ethyl]imidazo[1,2-a]pyrazin-2-yl]carbamoyl]indazol-4-yl]-4-piperidyl]carbamate (Intermediate F16, 33 mg, 54.66 μmol) in DCM (0.5 mL) was added TFA (0.25 mL) dropwise at room temperature. The resulting mixture was stirred for 1 h at room temperature. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 35% to 75% gradient in 14 min; detector, UV 254 nm) to afford 4-[4-(ethylamino)-1-piperidyl]-2-methyl-N-[6-methyl-8-[2-(methylamino)-2-oxo-ethyl]imidazo[1,2-a]pyrazin-2-yl]indazole-7-carboxamide (Compound 872, 9 mg, 17.87 μmol). LCMS (ES, m/z): 504 [M+H]⁺. ¹H NMR (300 MHz, DMSO-d₆) δ 11.42 (s, 1H), 8.79 (s, 1H), 8.38 (d, J=1.1 Hz, 1H), 8.36 (s, 1H), 8.03 (d, J=8.2 Hz, 1H), 8.00 (d, J=4.7 Hz, 1H), 6.51 (d, J=8.3 Hz, 1H), 4.30 (s, 3H), 3.92 (d, J=17.7 Hz, 4H), 3.08 (t, J=11.8 Hz, 2H), 2.76-2.56 (m, 6H), 2.40 (d, J=0.9 Hz, 3H), 1.97 (d, J=12.6 Hz, 2H), 1.45 (q, J=10.5, 9.9 Hz, 2H), 1.05 (t, J=7.1 Hz, 3H).

### Example 287. Synthesis of Compound 873

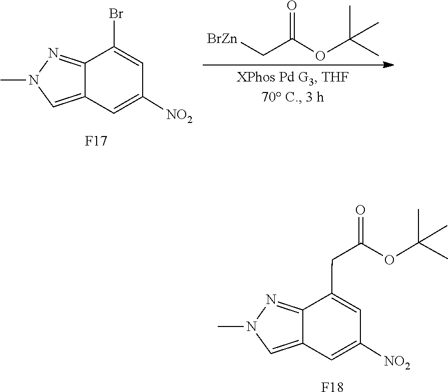

To a stirred mixture of 7-bromo-2-methyl-5-nitro-indazole (Intermediate F17, 2 g, 7.81 mmol) in THF (30 mL) were added bromo-(2-tert-butoxy-2-oxo-ethyl) zinc (6.10 g, 23.43 mmol) and XPhos Pd G3 (661.1 mg, 781.08 μmol) at rt under N₂. The mixture was stirred for 3 h at 70° C. The reaction was quenched with ice water (100 mL) and EA (100 mL). The resulting mixture was filtrated and the filtrate was extracted with EA (2×100 mL). The combined organic layers were washed with brine (2×150 mL), dried over anhydrous Na₂SO₄. After filtration, the filtrate was concentrated under reduced pressure. The residue was purified by silica gel column chromatography, eluted with PE/EA (2/1) to afford tert-butyl 2-(2-methyl-5-nitro-indazol-7-yl)acetate (Intermediate F18, 1.67 g, 5.73 mmol). LCMS (ES, m/z): 292 [M+H]⁺.

### Synthesis of Intermediate F19

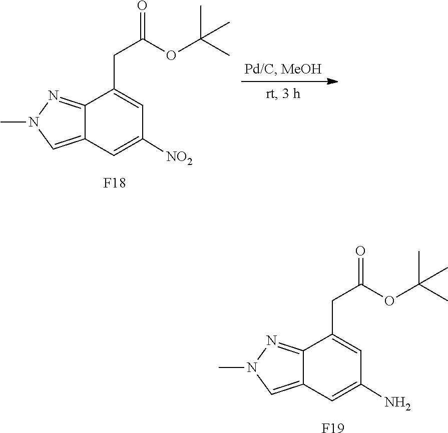

To a solution of tert-butyl 2-(2-methyl-5-nitro-indazol-7-yl)acetate (Intermediate F18, 1.5 g, 5.15 mmol) in MeOH (30 mL) was added Pd/C (10% on Carbon (wetted with ca. 55% water)) (547.99 mg, 5.15 mmol) under N₂. The mixture was stirred for 3 h at rt under H₂. The mixture was filtered through a Celite pad and concentrated under reduced pressure to afford tert-butyl 2-(5-amino-2-methyl-indazol-7-yl)acetate (Intermediate F19, 1.29 g, 4.94 mmol). LCMS (ES, m/z): 262 [M+H]⁺.

### Synthesis of Intermediate F21

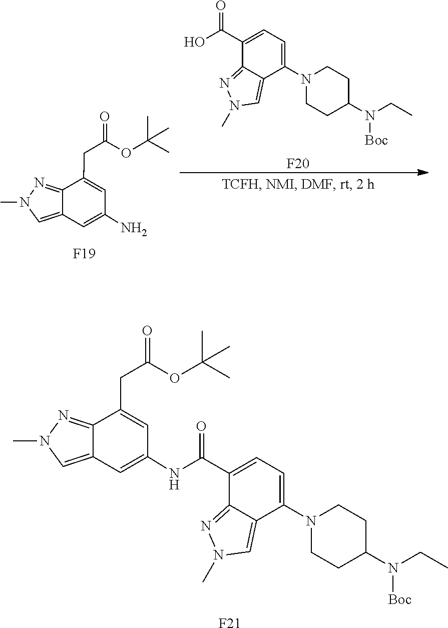

To a solution of 4-[4-[tert-butoxycarbonyl(ethyl)amino]-1-piperidyl]-2-methyl-indazole-7-carboxylic acid (Intermediate F20, 600 mg, 1.49 mmol) in MeCN (6 mL) were added tert-butyl 2-(5-amino-2-methyl-indazol-7-yl)acetate (Intermediate F19, 428.5 mg, 1.64 mmol), NMI (489.6 mg, 5.96 mmol) and TCFH (627.4 mg, 2.24 mmol) at rt. The mixture was stirred for 2 h at rt. The reaction was diluted with H₂O and the precipitated solids were collected by filtration. The filter cake was purified by trituration with Ethyl ether to afford tert-butyl 2-[5-[[4-[4-[tert-butoxycarbonyl(ethyl)amino]-1-piperidyl]-2-methyl-indazole-7-carbonyl]amino]-2-methyl-indazol-7-yl]acetate (Intermediate F21, 630 mg, 975.55 μmol, 65.44% yield) as a yellow solid. LCMS (ES, m/z): 646 [M+H]⁺.

### Synthesis of Intermediate F22

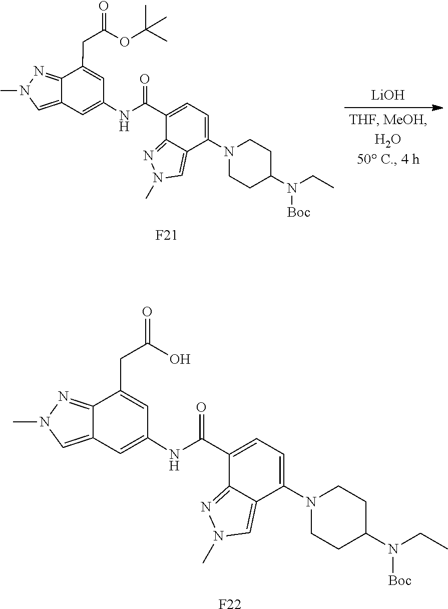

To a solution of tert-butyl 2-[5-[[4-[4-[tert-butoxycarbonyl(ethyl)amino]-1-piperidyl]-2-methyl-indazole-7-carbonyl]amino]-2-methyl-indazol-7-yl]acetate (Intermediate F21, 600 mg, 929.10 μmol) in THF (2.5 mL)/MeOH (2.5 mL)/H₂O (2.5 mL) was added LiOH H₂O (194.9 mg, 4.65 mmol) at rt. The mixture was stirred for 4 h at 50° C. The resulting mixture was diluted with H₂O and adjusted to pH 5 with 1 N HCl. The resulting mixture was extracted with DCM/MeOH=4/1 (4×20 mL). The combined organic layers were washed with brine (40 mL), dried over anhydrous Na₂SO₄. The resulting mixture was concentrated under reduced pressure to afford 2-[5-[[4-[4-[tert-butoxycarbonyl(ethyl)amino]-1-piperidyl]-2-methyl-indazole-7-carbonyl]amino]-2-methyl-indazol-7-yl]acetic acid (Intermediate F22, 420 mg, 712.25 μmol) as a yellow solid. LCMS (ES, m/z): 590 [M+H]⁺.

### Synthesis of Intermediate F23

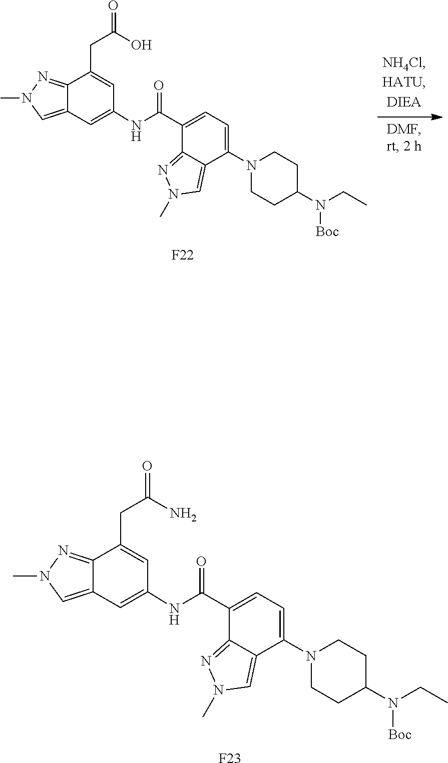

To a solution of 2-[5-[[4-[4-[tert-butoxycarbonyl(ethyl)amino]-1-piperidyl]-2-methyl-indazole-7-carbonyl]amino]-2-methyl-indazol-7-yl]acetic acid (Intermediate F22, 250 mg, 423.96 μmol) in DMF (3 mL) were added NH₄Cl (90.7 mg, 1.70 mmol), DIEA (219.1 mg, 1.70 mmol) and HATU (241.8 mg, 635.93 μmol) at rt. The mixture was stirred for 2 h at rt. The reaction was diluted with H₂O (15 mL). The resulting mixture was extracted with EA (2×15 mL). The combined organic layers were washed with H₂O (2×20 mL), dried over anhydrous Na₂SO₄. After filtration, the filtrate was concentrated under reduced pressure. The residue was purified by silica gel column chromatography, eluted with DCM/MeOH (19/1) to afford tert-butyl N-[1-[7-[[7-(2-amino-2-oxo-ethyl)-2-methyl-indazol-5-yl]carbamoyl]-2-methyl-indazol-4-yl]-4-piperidyl]-N-ethyl-carbamate (Intermediate F23, 150 mg, 254.80 μmol). LCMS (ES, m/z): 589 [M+H]⁺.

### Synthesis of Compound 873

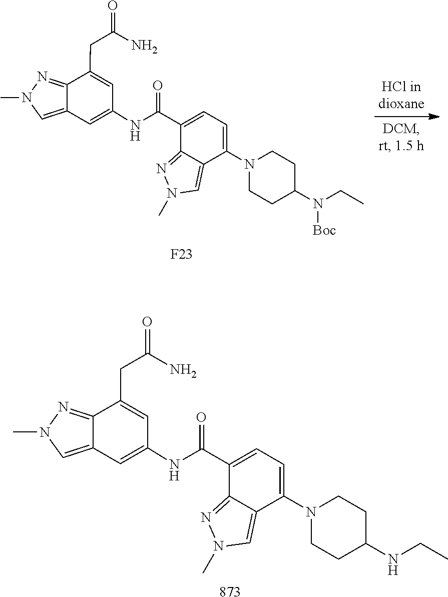

To a solution of tert-butyl N-[1-[7-[[7-(2-amino-2-oxo-ethyl)-2-methyl-indazol-5-yl]carbamoyl]-2-methyl-indazol-4-yl]-4-piperidyl]-N-ethyl-carbamate (Intermediate F23, 40 mg, 67.95 μmol) in DCM (0.5 mL) was added HCl (4.0 M in 1,4-dioxane) (169.87 μL). The mixture was stirred for 1.5 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 35% to 55% gradient in 7 min; detector, UV 254 nm) to afford N-[7-(2-amino-2-oxo-ethyl)-2-methyl-indazol-5-yl]-4-[4-(ethylamino)-1-piperidyl]-2-methyl-indazole-7-carboxamide (Compound 873, 12 mg, 24.56 μmol). LCMS (ES, m/z): 489 [M+H]⁺. ¹H NMR (400 MHz, DMSO-d₆) δ 11.08 (s, 1H), 8.75 (s, 1H), 8.28 (d, J=2.7 Hz, 2H), 7.99 (d, J=8.0 Hz, 1H), 7.44 (s, 1H), 7.23 (s, 1H), 6.96 (s, 1H), 6.49 (d, J=8.1 Hz, 1H), 4.30 (s, 3H), 4.16 (s, 3H), 3.87 (d, J=12.6 Hz, 2H), 3.76 (s, 2H), 3.03 (t, J=11.7 Hz, 2H), 2.64 (dd, J=17.2, 10.0 Hz, 3H), 2.01-1.92 (m, 2H), 1.46 (q, J=11.2 Hz, 2H), 1.05 (t, J=7.1 Hz, 3H).

### Example 288. Synthesis of Compound 885

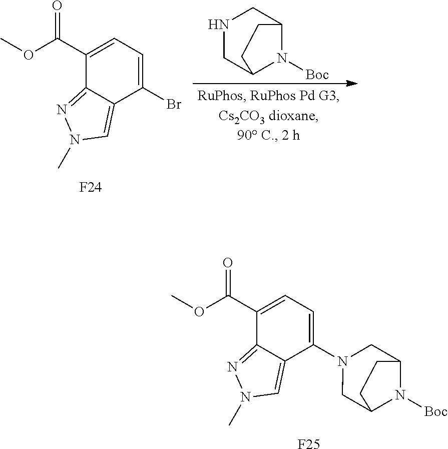

To a stirred mixture of methyl 4-bromo-2-methyl-indazole-7-carboxylate (Intermediate F24, 160 mg, 594.59 μmol) in dioxane (2 mL) were added tert-butyl 3,8-diazabicyclo[3.2.1]octane-8-carboxylate (151.4 mg, 713.50 μmol), Cs₂CO₃ (387.4 mg, 1.19 mmol), Ruphos (55.5 mg, 118.92 μmol) and RuPhos Pd G3 (49.7 mg, 59.46 μmol) at rt under N₂. The mixture was stirred for 2 h at 90° C. The reaction was diluted with H₂O (15 mL). The resulting mixture was extracted with DCM (2×15 mL). The combined organic layers were washed with H₂O (20 mL), dried over anhydrous Na₂SO₄. After filtration, the filtrate was concentrated under reduced pressure. The residue was purified by silica gel column chromatography, eluted with DCM/MeOH (20/1) to afford methyl 4-(8-tert-butoxycarbonyl-3,8-diazabicyclo[3.2.1]octan-3-yl)-2-methyl-indazole-7-carboxylate (Intermediate F25, 230 mg, 574.32 μmol). LCMS (ES, m/z): 401 [M+H]⁺.

### Synthesis of Intermediate F26

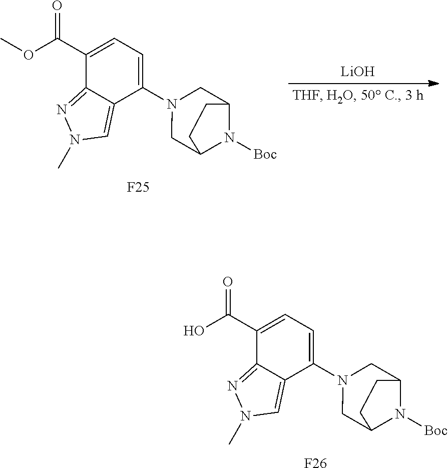

To a solution of methyl 4-(8-tert-butoxycarbonyl-3,8-diazabicyclo[3.2.1]octan-3-yl)-2-methyl-indazole-7-carboxylate (Intermediate F25, 250 mg, 624.27 μmol) in THF (2 mL)/H₂O (1 mL) was added LiOH·H₂O (130.9 mg, 3.12 mmol) at rt. The mixture was stirred for 3 h at 50° C. The resulting mixture was diluted with H₂O and adjusted to pH 5-6 with 1M HCl. The resulting mixture was extracted with DCM (2×15 mL). The combined organic layers were washed with H₂O (20 mL), dried over anhydrous Na₂SO₄. The resulting mixture was concentrated under reduced pressure to afford 4-(8-tert-butoxycarbonyl-3,8-diazabicyclo[3.2.1]octan-3-yl)-2-methyl-indazole-7-carboxylic acid (Intermediate F26, 240 mg, 621.05 μmol). LCMS (ES, m/z): 387 [M+H]⁺.

### Synthesis of Intermediate F27

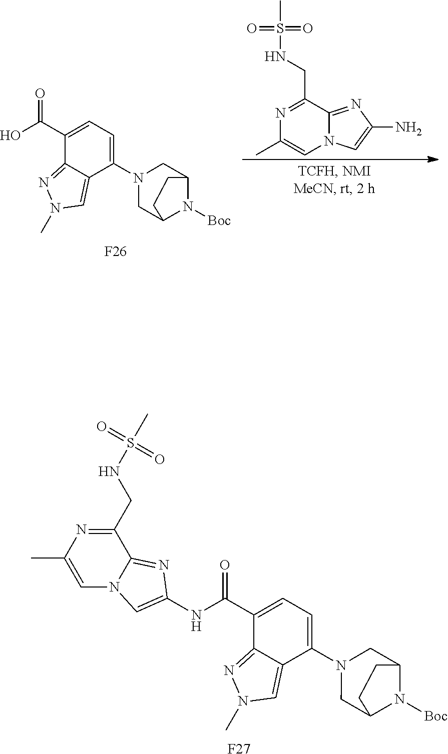

To a solution of 4-(8-tert-butoxycarbonyl-3,8-diazabicyclo[3.2.1]octan-3-yl)-2-methyl-indazole-7-carboxylic acid (Intermediate F26, 45 mg, 116.45 μmol) in MeCN (0.5 mL) were added N-[(2-amino-6-methyl-imidazo[1,2-a]pyrazin-8-yl)methyl]methanesulfonamide (32.7 mg, 128.09 μmol), NMI (38.2 mg, 465.79 μmol) and TCFH (49.0 mg, 174.67 μmol) at rt. The mixture was stirred for 2 h at rt. The reaction was diluted with H₂O and the precipitated solids were collected by filtration. The filter cake was purified by trituration with Ethyl ether to afford tert-butyl 3-[7-[[8-(methanesulfonamidomethyl)-6-methyl-imidazo[1,2-a]pyrazin-2-yl]carbamoyl]-2-methyl-indazol-4-yl]-3,8-diazabicyclo[3.2.1]octane-8-carboxylate (Intermediate F27, 45 mg, 72.15 μmol). LCMS (ES, m/z): 624 [M+H]⁺.

### Synthesis of Compound 885

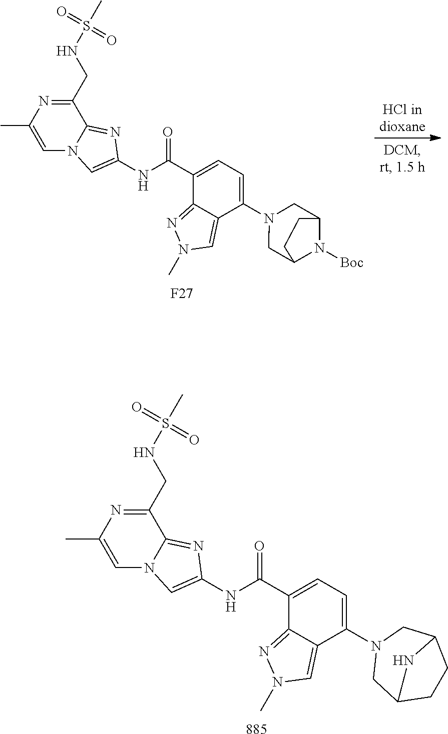

To a solution of tert-butyl 3-[7-[[8-(methanesulfonamidomethyl)-6-methyl-imidazo[1,2-a]pyrazin-2-yl]carbamoyl]-2-methyl-indazol-4-yl]-3,8-diazabicyclo[3.2.1]octane-8-carboxylate (Intermediate F27, 45 mg, 72.15 μmol) in DCM (0.5 mL) was added HCl (4.0 M in 1,4-dioxane) (180 μL). The mixture was stirred for 1.5 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 30% to 55% gradient in 7 min; detector, UV 254 nm) to afford 4-(3,8-diazabicyclo[3.2.1]octan-3-yl)-N-[8-(methanesulfonamidomethyl)-6-methyl-imidazo[1,2-a]pyrazin-2-yl]-2-methyl-indazole-7-carboxamide (Compound 885, 22 mg, 42.02 μmol). LCMS (ES, m/z): 524 [M+H]⁺. ¹H NMR (400 MHz, DMSO-d₆) δ 11.54 (s, 1H), 8.88 (s, 1H), 8.43 (d, J=1.2 Hz, 1H), 8.40 (s, 1H), 8.01 (d, J=8.3 Hz, 1H), 7.49 (t, J=5.5 Hz, 1H), 6.38 (d, J=8.4 Hz, 1H), 4.67 (d, J=5.2 Hz, 2H), 4.28 (s, 3H), 3.72 (dd, J=11.4, 2.4 Hz, 2H), 3.56 (d, J=3.9 Hz, 2H), 3.16 (dd, J=11.4, 2.3 Hz, 2H), 3.07 (s, 3H), 2.46-2.42 (m, 3H), 1.77 (dt, J=11.9, 8.0 Hz, 4H).

### Example 289. Synthesis of Compound 887

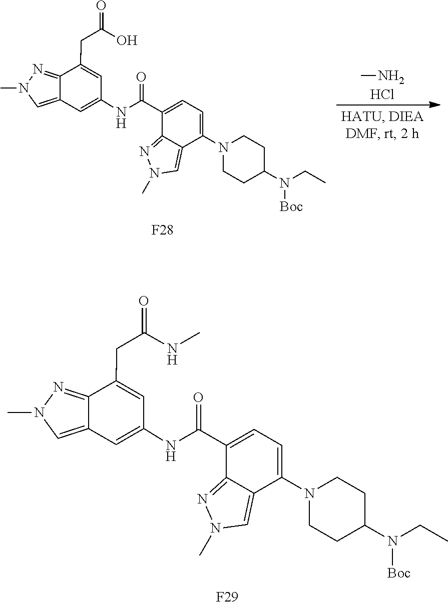

To a solution of 2-[5-[[4-[4-[tert-butoxycarbonyl(ethyl)amino]-1-piperidyl]-2-methyl-indazole-7-carbonyl]amino]-2-methyl-indazol-7-yl]acetic acid (Intermediate F28, 40 mg, 67.83 μmol) in DMF (0.5 mL) were added methanamine hydrochloride (9.1 mg, 135.67 μmol), DIEA (43.8 mg, 339.16 μmol) and HATU (38.7 mg, 101.75 μmol) at rt. The mixture was stirred for 2 h at rt. The reaction was diluted with H₂O (10 mL). The resulting mixture was extracted with EA (2×10 mL). The combined organic layers were washed with H₂O (2×10 mL), dried over anhydrous Na₂SO₄. After filtration, the filtrate was concentrated under reduced pressure. The residue was purified by silica gel column chromatography, eluted with DCM/MeOH (13/1) to afford tert-butyl N-ethyl-N-[1-[2-methyl-7-[[2-methyl-7-[[2-(methylamino)-2-oxo-ethyl]indazol-5-yl]carbamoyl]indazol-4-yl]-4-piperidyl]carbamate (Intermediate F29, 33 mg, 54.75 μmol). LCMS (ES, m/z): 603 [M+H]⁺.

### Synthesis of Compound 887

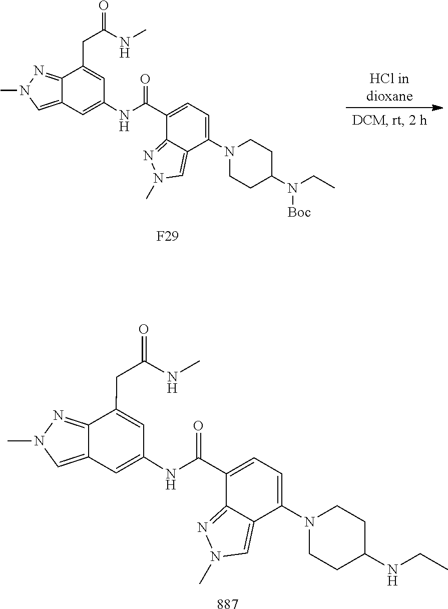

To a solution of tert-butyl N-ethyl-N-[1-[2-methyl-7-[[2-methyl-7-[2-(methylamino)-2-oxo-ethyl]indazol-5-yl]carbamoyl]indazol-4-yl]-4-piperidyl]carbamate (Intermediate F29, 30 mg, 49.77 μmol) in DCM (0.5 mL) was added HCl (4.0 M in 1,4-dioxane) (4 M, 124.43 μL). The mixture was stirred for 2 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 30% to 50% gradient in 7 min; detector, UV 254 nm) to afford 4-[4-(ethylamino)-1-piperidyl]-2-methyl-N-[2-methyl-7-[2-(methylamino)-2-oxo-ethyl]indazol-5-yl]indazole-7-carboxamide (Compound 887, 12 mg, 23.88 μmol). LCMS (ES, m/z): 503 [M+H]⁺. ¹H NMR (400 MHz, DMSO-d₆) δ 11.06 (s, 1H), 8.73 (s, 1H), 8.29-8.23 (m, 2H), 7.96 (d, J=8.1 Hz, 1H), 7.88 (d, J=4.8 Hz, 1H), 7.17 (d, J=1.9 Hz, 1H), 6.46 (d, J=8.2 Hz, 1H), 4.27 (s, 3H), 4.13 (s, 3H), 3.84 (d, J=12.7 Hz, 2H), 3.75 (s, 2H), 3.01 (t, J=11.6 Hz, 2H), 2.70-2.54 (m, 6H), 1.94 (d, J=12.5 Hz, 2H), 1.44 (tt, J=13.3, 7.3 Hz, 3H), 1.02 (t, J=7.1 Hz, 3H).

### Example 290. Synthesis of Compound 888

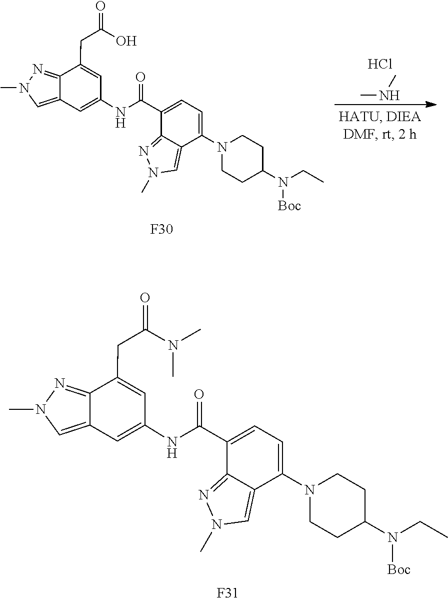

To a solution of 2-[5-[[4-[4-[tert-butoxycarbonyl(ethyl)amino]-1-piperidyl]-2-methyl-indazole-7-carbonyl]amino]-2-methyl-indazol-7-yl]acetic acid (Intermediate F30, 40 mg, 67.83 μmol) in DMF (0.5 mL) were added dimethylamine hydrochloride (11.0 mg, 135.67 μmol), DIEA (43.8 mg, 339.16 μmol) and HATU (38.7 mg, 101.75 μmol) at rt. The mixture was stirred for 2 h at rt. The reaction was diluted with H₂O (10 mL). The resulting mixture was extracted with EA (2×10 mL). The combined organic layers were washed with H₂O (2×10 mL), dried over anhydrous Na₂SO₄. After filtration, the filtrate was concentrated under reduced pressure. The residue was purified by silica gel column chromatography, eluted with DCM/MeOH (16/1) to afford tert-butyl N-[1-[7-[[7-[2-(dimethylamino)-2-oxo-ethyl]-2-methyl-indazol-5-yl]carbamoyl]-2-methyl-indazol-4-yl]-4-piperidyl]-N-ethyl-carbamate (Intermediate F31, 24 mg, 38.91 μmol). LCMS (ES, m/z): 617 [M+H]⁺.

### Synthesis of Compound 888

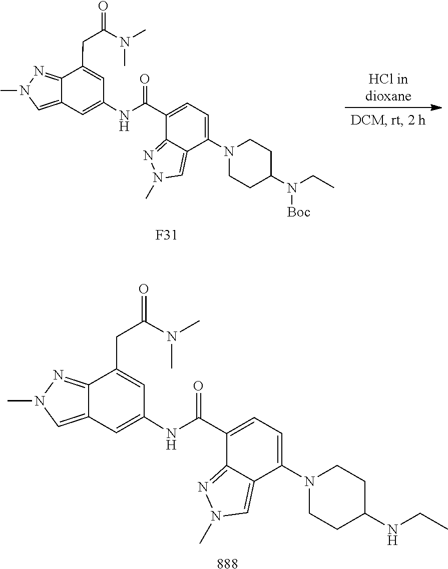

To a solution of tert-butyl N-[1-[7-[[7-[2-(dimethylamino)-2-oxo-ethyl]-2-methyl-indazol-5-yl]carbamoyl]-2-methyl-indazol-4-yl]-4-piperidyl]-N-ethyl-carbamate (Intermediate F31, 24 mg, 38.91 μmol) in DCM (0.3 mL) was added HCl (4.0 M in 1,4-dioxane) (100 μL). The mixture was stirred for 2 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 35% to 55% gradient in 7 min; detector, UV 254 nm) to afford N-[7-[2-(dimethylamino)-2-oxo-ethyl]-2-methyl-indazol-5-yl]-4-[4-(ethylamino)-1-piperidyl]-2-methyl-indazole-7-carboxamide (Compound 888, 9 mg, 17.42 μmol). LCMS (ES, m/z): 517 [M+H]⁺. ¹H NMR (400 MHz, DMSO-d₆) δ 11.05 (s, 1H), 8.73 (s, 1H), 8.28-8.21 (m, 2H), 7.96 (d, J=8.1 Hz, 1H), 7.10 (d, J=1.9 Hz, 1H), 6.46 (d, J=8.2 Hz, 1H), 4.27 (s, 3H), 4.13 (s, 3H), 3.96 (s, 2H), 3.84 (d, J=12.6 Hz, 2H), 3.09 (s, 3H), 3.07-2.95 (m, 2H), 2.87 (s, 3H), 2.71-2.54 (m, 3H), 1.94 (d, J=12.5 Hz, 2H), 1.55-1.34 (m, 3H), 1.02 (t, J=7.1 Hz, 3H).

### Example 291. Synthesis of Compound 889

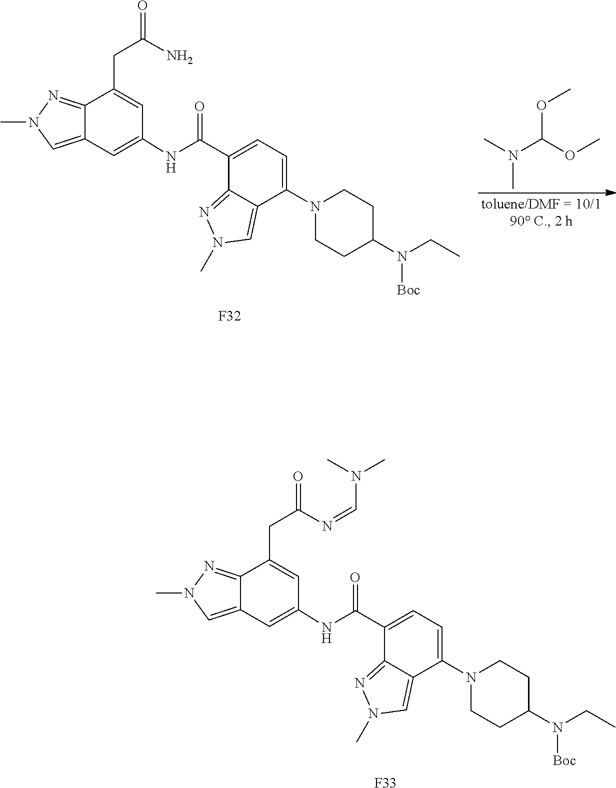

To a solution of tert-butyl N-[1-[7-[[7-(2-amino-2-oxo-ethyl)-2-methyl-indazol-5-yl]carbamoyl]-2-methyl-indazol-4-yl]-4-piperidyl]-N-ethyl-carbamate (Intermediate F32, 105 mg, 178.36 μmol) in toluene (1 mL)/DMF (0.1 mL) was added 1,1-dimethoxy-N,N-dimethyl-methanamine (63.7 mg, 535.08 μmol) at rt. The mixture was stirred for 2 h at 90° C. The resulting mixture was concentrated under reduced pressure to afford tert-butyl N-[1-[7-[[7-[2-[(Z)-dimethylaminomethyleneamino]-2-oxo-ethyl]-2-methyl-indazol-5-yl]carbamoyl]-2-methyl-indazol-4-yl]-4-piperidyl]-N-ethyl-carbamate (Intermediate F33, 110 mg, crude). The crude product was used for next step without further purification. LCMS (ES, m/z): 644 [M+H]⁺.

### Synthesis of Intermediate F34

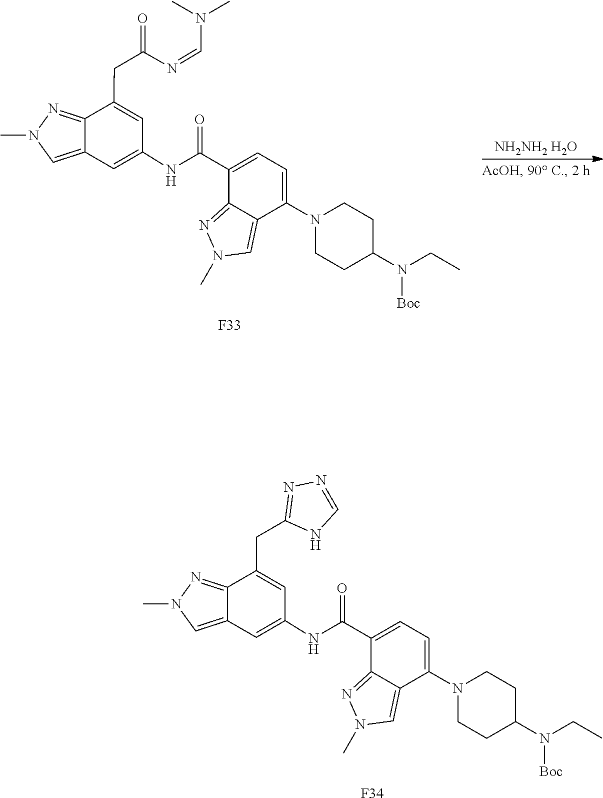

To a solution of tert-butyl N-[1-[7-[[7-[2-[(Z)-dimethylaminomethyleneamino]-2-oxo-ethyl]-2-methyl-indazol-5-yl]carbamoyl]-2-methyl-indazol-4-yl]-4-piperidyl]-N-ethyl-carbamate (Intermediate F33, 105 mg, 163.10 μmol) in AcOH (1.1 mL) was added NH₂NH₂·H₂O (25.0 mg, 489.30 μmol, 98% purity) at rt. The mixture was stirred for 2 h at 90° C. The resulting mixture was diluted with H₂O (2 ml) and adjusted to pH 8 with NaHCO₃ (aq.). Then extracted with DCM (2×10 mL). The combined organic layers were washed with H₂O (10 mL), dried over anhydrous Na₂SO₄. After filtration, the filtrate was concentrated under reduced pressure. The residue was purified by silica gel column chromatography, eluted with DCM/MeOH (12/1) to afford tert-butyl N-ethyl-N-[1-[2-methyl-7-[[2-methyl-7-(4H-1,2,4-triazol-3-ylmethyl) indazol-5-yl]carbamoyl]indazol-4-yl]-4-piperidyl]carbamate (Intermediate F34, 35 mg, 57.12 μmol). LCMS (ES, m/z): 613 [M+H]⁺.

### Synthesis of Compound 889

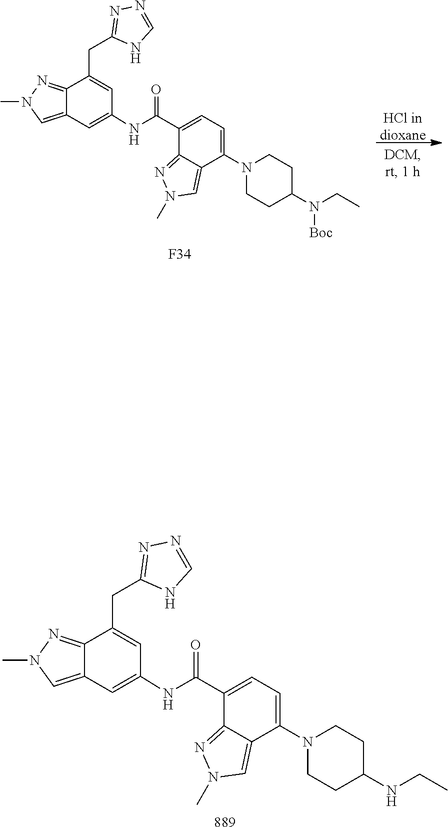

To a solution of tert-butyl N-ethyl-N-[1-[2-methyl-7-[[2-methyl-7-(4H-1,2,4-triazol-3-ylmethyl) indazol-5-yl]carbamoyl]indazol-4-yl]-4-piperidyl]carbamate (Intermediate F34, 35 mg, 57.12 μmol) in DCM (0.5 mL) was added HCl (4.0 M in 1,4-dioxane) (150 μL). The mixture was stirred for 1 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 35% to 50% gradient in 7 min; detector, UV 254 nm) to afford 4-[4-(ethylamino)-1-piperidyl]-2-methyl-N-[2-methyl-7-(4H-1,2,4-triazol-3-ylmethyl) indazol-5-yl]indazole-7-carboxamide (Compound 889, 13 mg, 25.36 μmol). LCMS (ES, m/z): 513 [M+H]⁺. ¹H NMR (300 MHz, DMSO-d₆) δ 14.09-13.44 (m, 1H), 11.07 (s, 1H), 8.73 (s, 1H), 8.31-8.21 (m, 2H), 8.14 (s, 1H), 7.94 (d, J=8.0 Hz, 1H), 7.05 (s, 1H), 6.45 (d, J=8.2 Hz, 1H), 4.36 (s, 2H), 4.24 (s, 3H), 4.14 (s, 3H), 3.83 (d, J=12.5 Hz, 2H), 3.00 (t, J=11.6 Hz, 2H), 2.59 (q, J=7.1 Hz, 3H), 1.94 (d, J=12.7 Hz, 2H), 1.42 (q, J=11.0 Hz, 2H), 1.02 (t, J=7.1 Hz, 3H).

### Example 292. Synthesis of Compound 894

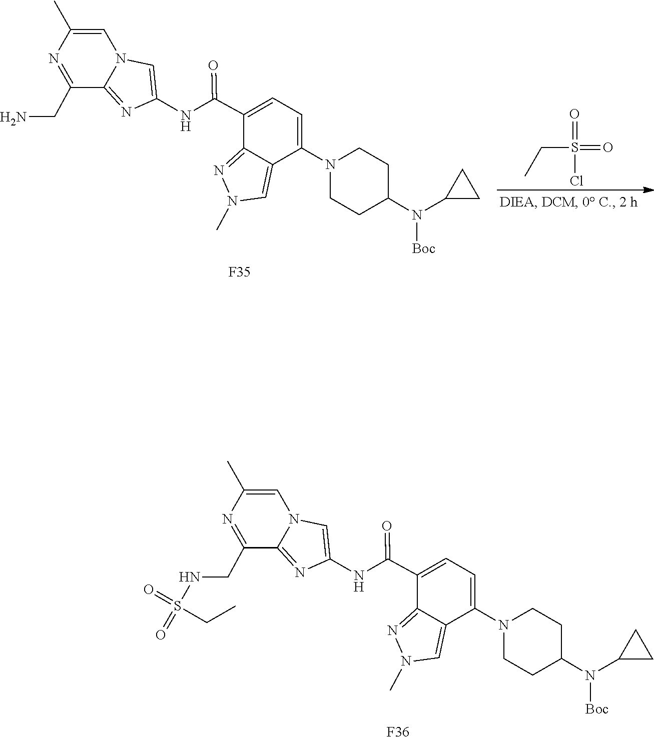

To a solution of tert-butyl N-[1-[7-[[8-(aminomethyl)-6-methyl-imidazo[1,2-a]pyrazin-2-yl]carbamoyl]-2-methyl-indazol-4-yl]-4-piperidyl]-N-cyclopropyl-carbamate (Intermediate F35, 50 mg, 87.16 μmol) in DCM (1 mL) were added DIEA (22.5 mg, 174.31 μmol) and ethanesulfonyl chloride (12.3 mg, 95.87 μmol) at 0° C. The mixture was stirred for 2 h at 0° C. The reaction was diluted with H₂O (10 mL). The resulting mixture was extracted with DCM (2×10 mL). The combined organic layers were washed with H₂O (10 mL), dried over anhydrous Na₂SO₄. After filtration, the filtrate was concentrated under reduced pressure. The residue was purified by silica gel column chromatography, eluted with DCM/EA (3/7) to afford tert-butyl N-cyclopropyl-N-[1-[7-[8-[(ethylsulfonylamino)methyl]-6-methyl-imidazo[1,2-a]pyrazin-2-yl]carbamoyl]-2-methyl-indazol-4-yl]-4-piperidyl]carbamate (Intermediate F36, 50 mg, 75.10 μmol, 86.16% yield) as a yellow solid. LCMS (ES, m/z): 666 [M+H]⁺.

### Synthesis of Compound 894

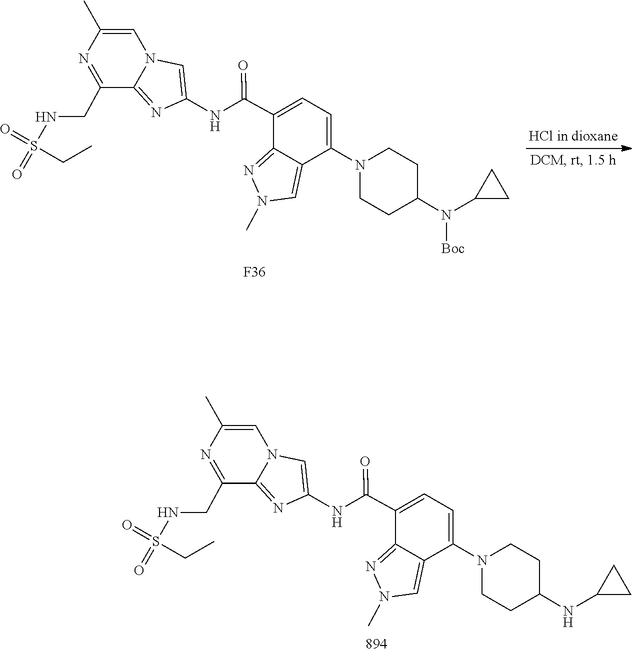

To a solution of tert-butyl N-cyclopropyl-N-[1-[7-[8-[(ethylsulfonylamino)methyl]-6-methyl-imidazo[1,2-a]pyrazin-2-yl]carbamoyl]-2-methyl-indazol-4-yl]-4-piperidyl]carbamate (Intermediate F36, 50 mg, 75.10 μmol) in DCM (1 mL) was added HCl (4.0 M in 1,4-dioxane) (187 μL). The mixture was stirred for 1.5 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 30% to 50% gradient in 7 min; detector, UV 254 nm) to afford 4-[4-(cyclopropylamino)-1-piperidyl]-N-[8-[(ethylsulfonylamino)methyl]-6-methyl-imidazo[1,2-a]pyrazin-2-yl]-2-methyl-indazole-7-carboxamide (Compound 894, 22 mg, 38.89 μmol, 51.79% yield) as an off-white solid. LCMS (ES, m/z): 566 [M+H]⁺. ¹H NMR (300 MHz, DMSO-d₆) δ 11.50 (s, 1H), 8.80 (s, 1H), 8.42 (d, J=9.8 Hz, 2H), 8.03 (d, J=8.1 Hz, 1H), 7.54 (t, J=5.9 Hz, 1H), 6.50 (d, J=8.3 Hz, 1H), 4.65 (d, J=5.5 Hz, 2H), 4.30 (s, 3H), 3.90 (d, J=12.7 Hz, 2H), 3.25-2.99 (m, 4H), 2.77 (d, J=9.1 Hz, 1H), 2.46-2.40 (m, 3H), 2.37-2.22 (m, 1H), 2.13 (tt, J=6.7, 3.6 Hz, 1H), 2.01 (d, J=12.7 Hz, 2H), 1.48 (q, J=10.8 Hz, 2H), 1.25 (t, J=7.3 Hz, 3H), 0.40 (dt, J=6.2, 3.0 Hz, 2H), 0.23 (p, J=3.9 Hz, 2H).

### Example 293. Synthesis of Compound 896

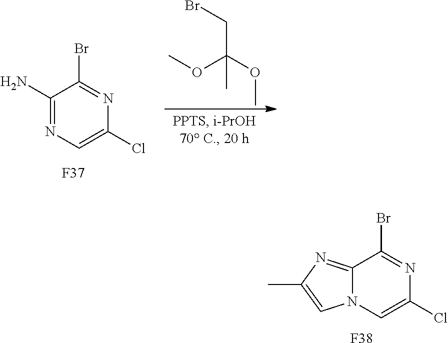

To a solution of 3-bromo-5-chloro-pyrazin-2-amine (Intermediate F37, 5 g, 23.99 mmol) in i-PrOH (75 mL) were added 1-bromo-2,2-dimethoxy-propane (13.17 g, 71.96 mmol) and PPTS (1.21 g, 4.80 mmol) at rt. The reaction was stirred for 20 h at 70° C. The reaction mixture was cooled to room temperature and concentrated under reduced pressure. The residue was purified by silica gel column chromatography, eluted with PE/THF (4/1) to afford 8-bromo-6-chloro-2-methyl-imidazo[1,2-a]pyrazine (Intermediate F38, 2.68 g, 10.87 mmol). LCMS (ES, m/z): 246 [M+H]⁺.

### Synthesis of Intermediate F39

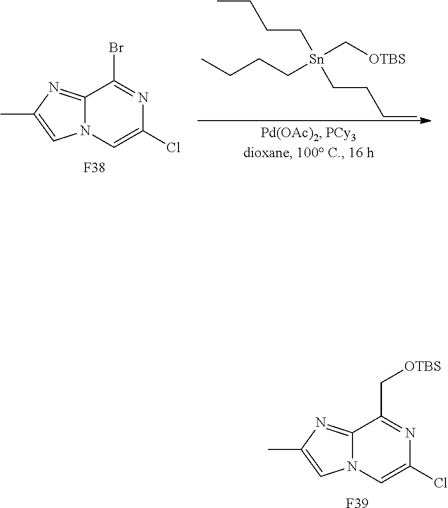

To a stirred mixture of 8-bromo-6-chloro-2-methyl-imidazo[1,2-a]pyrazine (Intermediate F38, 500 mg, 2.03 mmol) in dioxane (7.5 mL) were added tert-butyl-dimethyl-(tributylstannylmethoxy)silane (1.06 g, 2.43 mmol), PCy₃ (113.7 mg, 405.69 μmol) and Pd(OAc)₂ (45.5 mg, 202.85 μmol) at rt under N₂. The mixture was stirred for 16 h at 100° C. The reaction was quenched by sat. KF (aq.). The resulting mixture was extracted with DCM (2×30 mL). The combined organic layers were washed with brine (2×30 mL), dried over anhydrous Na₂SO₄. After filtration, the filtrate was concentrated under reduced pressure. The residue was purified by silica gel column chromatography, eluted with PE/THF (5/1) to afford tert-butyl-[(6-chloro-2-methyl-imidazo[1,2-a]pyrazin-8-yl)methoxy]-dimethyl-silane (Intermediate F39, 530 mg, 1.70 mmol). LCMS (ES, m/z): 312 [M+H]⁺.

### Synthesis of Intermediate F41

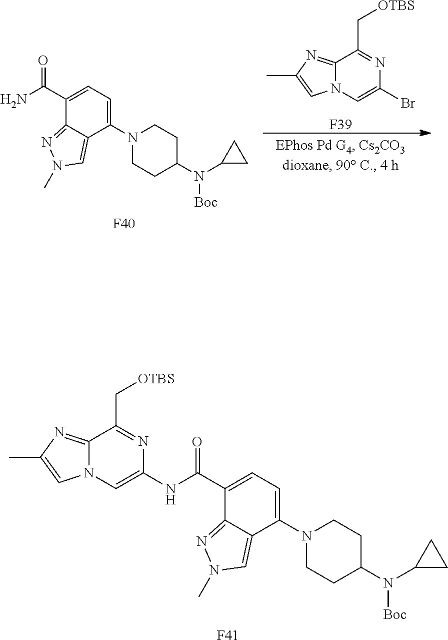

To a stirred mixture of tert-butyl N-[1-(7-carbamoyl-2-methyl-indazol-4-yl)-4-piperidyl]-N-cyclopropyl-carbamate (Intermediate F40, 100 mg, 241.83 μmol) in dioxane (1.5 mL) were added tert-butyl-[(6-chloro-2-methyl-imidazo[1,2-a]pyrazin-8-yl)methoxy]-dimethyl-silane (Intermediate F39, 90.5 mg, 290.20 μmol), Cs₂CO₃ (157.6 mg, 483.66 μmol) and EPhos Pd G4 (22.2 mg, 24.18 μmol) at rt under N₂. The mixture was stirred for 4 h at 90° C. The reaction was diluted with H₂O (15 mL). The resulting mixture was extracted with DCM (2×15 mL). The combined organic layers were washed with H₂O (15 mL), dried over anhydrous Na₂SO₄. After filtration, the filtrate was concentrated under reduced pressure. The residue was purified by silica gel column chromatography, eluted with PE/EA (1/1) to afford tert-butyl N-[1-[7-[[8-[[tert-butyl(dimethyl)silyl]oxymethyl]-2-methyl-imidazo[1,2-a]pyrazin-6-yl]carbamoyl]-2-methyl-indazol-4-yl]-4-piperidyl]-N-cyclopropyl-carbamate (Intermediate F41, 130 mg, 188.70 μmol). LCMS (ES, m/z): 689 [M+H]⁺.

### Synthesis of Compound 896

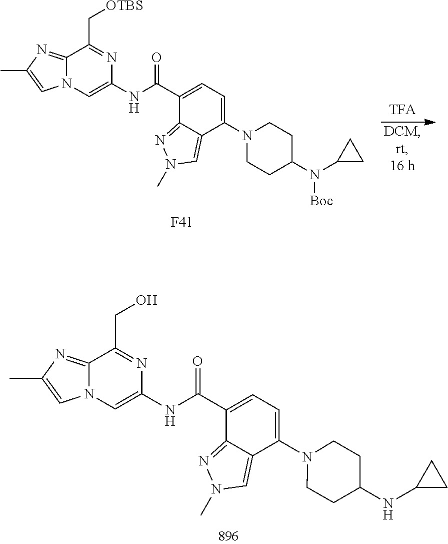

To a solution of tert-butyl N-[1-[7-[[8-[[tert-butyl(dimethyl)silyl]oxymethyl]-2-methyl-imidazo[1,2-a]pyrazin-6-yl]carbamoyl]-2-methyl-indazol-4-yl]-4-piperidyl]-N-cyclopropyl-carbamate (Intermediate F41, 65 mg, 94.35 μmol) in DCM (0.7 mL) was added TFA (200 μL). The mixture was stirred for 16 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₄HCO₃), 45% to 50% gradient in 7 min; detector, UV 254 nm) to afford 4-[4-(cyclopropylamino)-1-piperidyl]-N-[8-(hydroxymethyl)-2-methyl-imidazo[1,2-a]pyrazin-6-yl]-2-methyl-indazole-7-carboxamide (Compound 896, 10 mg, 21.07 μmol). LCMS (ES, m/z): 475 [M+H]⁺. ¹H NMR (300 MHz, DMSO-d₆) δ 11.30 (s, 1H), 9.37 (s, 1H), 8.79 (s, 1H), 8.02 (t, J=4.1 Hz, 2H), 6.50 (d, J=8.2 Hz, 1H), 5.37 (t, J=6.1 Hz, 1H), 4.89 (d, J=5.9 Hz, 2H), 4.28 (s, 3H), 3.89 (d, J=12.7 Hz, 2H), 3.07 (t, J=11.7 Hz, 2H), 2.85-2.70 (m, 1H), 2.40 (s, 3H), 2.12 (tt, J=6.7, 3.7 Hz, 1H), 2.00 (d, J=12.5 Hz, 2H), 1.47 (q, J=11.4 Hz, 2H), 0.39 (dt, J=6.2, 2.9 Hz, 2H), 0.23 (p, J=3.9 Hz, 2H).

### Example 294. Synthesis of Compound 897

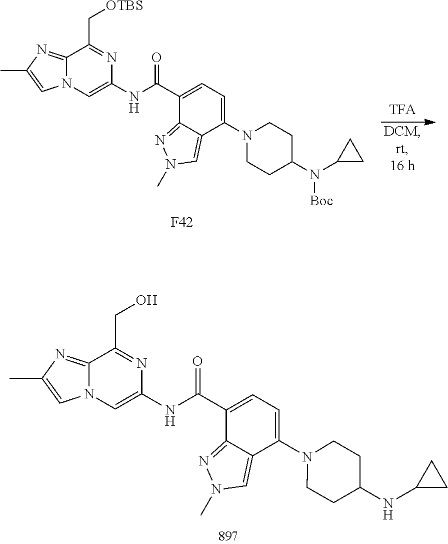

To a solution of tert-butyl N-[1-[7-[[8-[[tert-butyl(dimethyl)silyl]oxymethyl]-2-methyl-imidazo[1,2-a]pyrazin-6-yl]carbamoyl]-2-methyl-indazol-4-yl]-4-piperidyl]-N-cyclopropyl-carbamate (Intermediate F42, 65 mg, 94.35 μmol) in DCM (0.7 mL) was added TFA (322.7 mg, 2.83 mmol, 216.60 μL). The mixture was stirred for 16 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₄HCO₃), 35% to 45% gradient in 7 min; detector, UV 254 nm) to afford 4-[4-(allylamino)-1-piperidyl]-N-[8-(hydroxymethyl)-2-methyl-imidazo[1,2-a]pyrazin-6-yl]-2-methyl-indazole-7-carboxamide (Compound 897, 8.5 mg, 17.91 μmol, 18.98% yield). LCMS (ES, m/z): 475 [M+H]⁺. ¹H NMR (300 MHz, DMSO-d₆) δ 11.30 (s, 1H), 9.37 (s, 1H), 8.79 (s, 1H), 8.02 (t, J=4.1 Hz, 2H), 6.50 (d, J=8.2 Hz, 1H), 5.88 (ddt, J=16.4, 10.9, 5.7 Hz, 1H), 5.37 (t, J=6.1 Hz, 1H), 5.25-5.12 (m, 1H), 5.09-4.99 (m, 1H), 4.89 (d, J=6.0 Hz, 2H), 4.28 (s, 3H), 3.90 (d, J=12.8 Hz, 2H), 3.24 (d, J=5.8 Hz, 2H), 3.05 (t, J=11.8 Hz, 2H), 2.67 (d, J=9.6 Hz, 1H), 2.40 (s, 3H), 1.97 (d, J=12.6 Hz, 2H), 1.45 (q, J=11.0, 10.5 Hz, 2H).

### Example 295. Synthesis of Compound 900

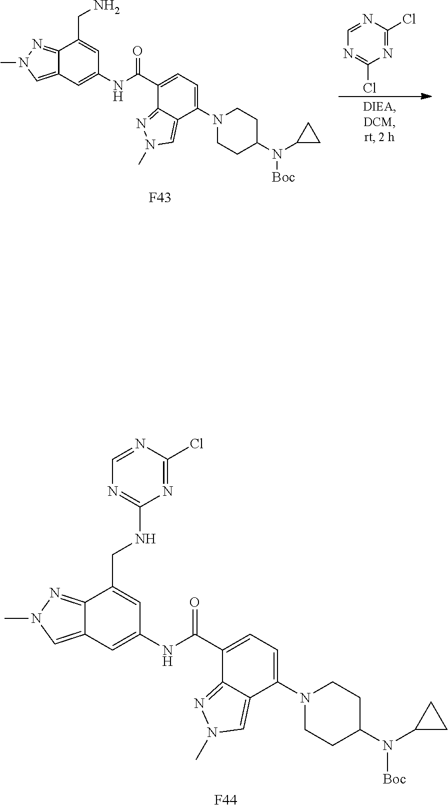

To a stirred solution of tert-butyl N-[1-[7-[[7-(aminomethyl)-2-methyl-indazol-5-yl]carbamoyl]-2-methyl-indazol-4-yl]-4-piperidyl]-N-cyclopropyl-carbamate (Intermediate F43, 90 mg, 157.15 μmol) and DIEA (60.9 mg, 471.45 μmol) in DCM (1 mL) was added 2,4-dichloro-1,3,5-triazine (25.9 mg, 172.87 μmol) at 0° C. under N₂ atmosphere. The resulting mixture was stirred for 2 h at rt. The resulting mixture was diluted with water (5 mL). The resulting mixture was extracted with EA (3×5 mL). The combined organic layers were washed brine (1×10 mL), dried over anhydrous Na₂SO₄. After filtration, the filtrate was concentrated under reduced pressure. The residue was purified by silica gel column chromatography, eluted with DCM/MeOH (1:10) to afford tert-butyl N-[1-[7-[[7-[[(4-chloro-1,3,5-triazin-2-yl)amino]methyl]-2-methyl-indazol-5-yl]carbamoyl]-2-methyl-indazol-4-yl]-4-piperidyl]-N-cyclopropyl-carbamate (Intermediate F44, 70 mg, 102.01 μmol). LCMS (ES, m/z): 686 [M+H]⁺.

### Synthesis of Compound 900

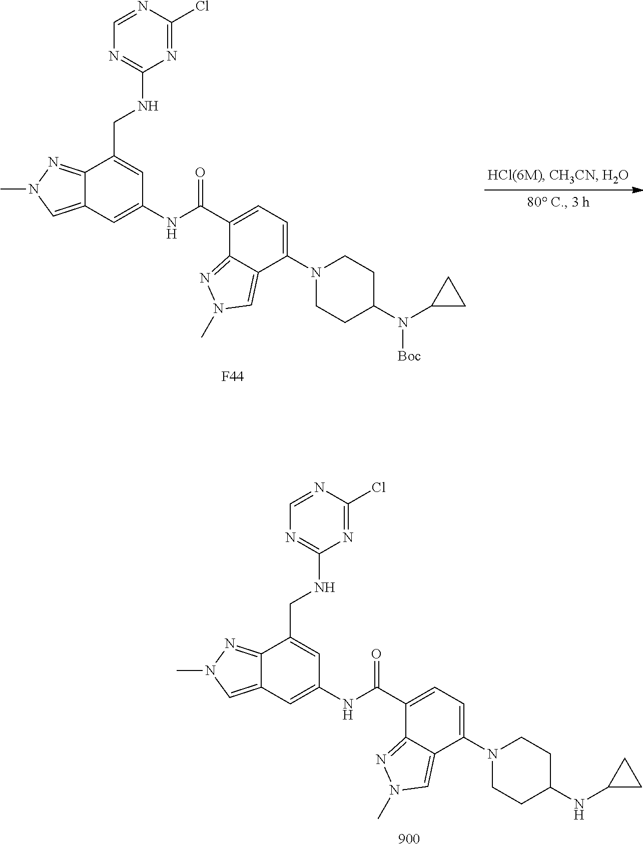

To a stirred solution of tert-butyl N-[1-[7-[[7-[[(4-chloro-1,3,5-triazin-2-yl)amino]methyl]-2-methyl-indazol-5-yl]carbamoyl]-2-methyl-indazol-4-yl]-4-piperidyl]-N-cyclopropyl-carbamate (Intermediate F44, 50 mg, 72.86 μmol) in MeCN (0.5 mL) and H₂O (0.5 mL) was added HCl (6 N, 0.5 mL) at rt. The resulting mixture was stirred for 3 h at 80° C. The resulting mixture was concentrated under reduced pressure. The crude product was purified by Prep-HPLC with the following conditions (SunFire Prep C18 OBD Column 19*150 mm, 5 μm 10 nm, mobile phase, MeCN in water (0.05% TFA), 20% to 40% gradient in 25 min; detector, UV 254 nm) to afford 4-(4-(cyclopropylamino)piperidin-1-yl)-N-(7-(((4-hydroxy-1,3,5-triazin-2-yl)amino)methyl)-2-methyl-2H-indazol-5-yl)-2-methyl-2H-indazole-7-carboxamide 2,2,2-trifluoroacetate (Compound 900, 10 mg, 14.67 μmol). LCMS (ES, m/z): 568 [M+H]⁺. ¹H NMR (300 MHz, DMSO-d₆) δ 11.66 (s, 1H), 11.13 (s, 1H), 8.83 (s, 1H), 8.78 (s, 2H), 8.51 (t, J=6.2 Hz, 1H), 8.33 (d, J=2.3 Hz, 1H), 8.28 (d, J=1.8 Hz, 1H), 8.14 (s, 1H), 8.00 (d, J=8.0 Hz, 1H), 7.19 (d, J=1.9 Hz, 1H), 6.55 (d, J=8.2 Hz, 1H), 4.80 (d, J=6.1 Hz, 2H), 4.25 (d, J=3.6 Hz, 3H), 4.19 (d, J=2.8 Hz, 3H), 4.03 (d, J=12.9 Hz, 2H), 3.03 (t, J=12.4 Hz, 2H), 2.89-2.71 (m, 1H), 2.21 (d, J=12.2 Hz, 2H), 1.76 (q, J=11.9 Hz, 2H), 0.85 (d, J=7.0 Hz, 4H).

### Example 296. Synthesis of Compound 904

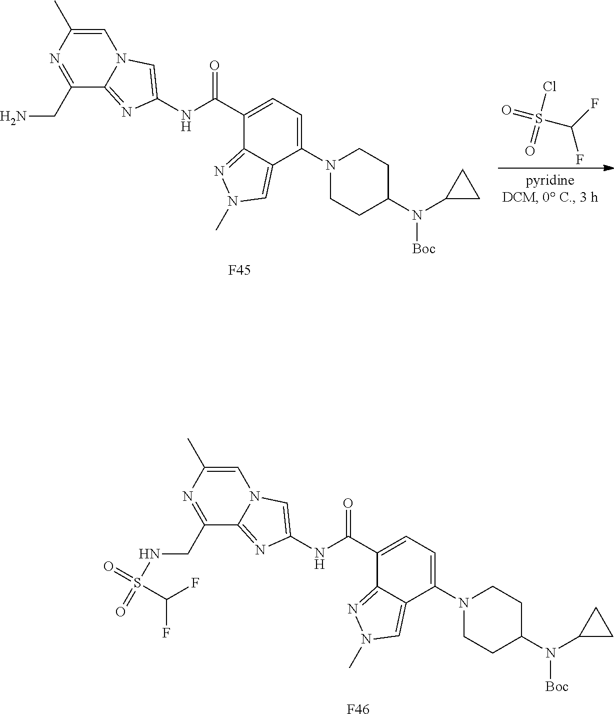

To a solution of tert-butyl N-[1-[7-[[8-(aminomethyl)-6-methyl-imidazo[1,2-a]pyrazin-2-yl]carbamoyl]-2-methyl-indazol-4-yl]-4-piperidyl]-N-cyclopropyl-carbamate (Intermediate F45, 60 mg, 104.59 μmol) in DCM (1 mL) were added Pyridine (24.8 mg, 313.76 μmol) and difluoromethanesulfonyl chloride (17.3 mg, 115.05 μmol) at 0° C. The mixture was stirred for 3 h at 0° C. The reaction was diluted with H₂O (10 mL). The resulting mixture was extracted with DCM (2×10 mL). The combined organic layers were washed with H₂O (10 mL), dried over anhydrous Na₂SO₄. After filtration, the filtrate was concentrated under reduced pressure. The residue was purified by silica gel column chromatography, eluted with DCM/MeOH (19/1) to afford tert-butyl N-cyclopropyl-N-[1-[7-[[8-[(difluoromethylsulfonylamino)methyl]-6-methyl-imidazo[1,2-a]pyrazin-2-yl]carbamoyl]-2-methyl-indazol-4-yl]-4-piperidyl]carbamate (Intermediate F46, 35 mg, 50.89 μmol). LCMS (ES, m/z): 688 [M+H]⁺.

### Synthesis of Compound 904

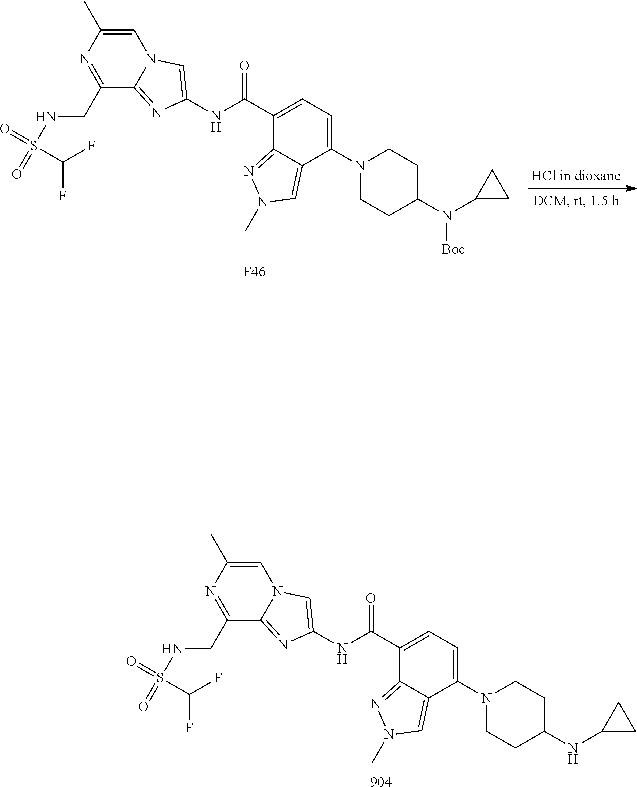

To a solution of tert-butyl N-cyclopropyl-N-[1-[7-[[8-[(difluoromethylsulfonylamino)methyl]-6-methyl-imidazo[1,2-a]pyrazin-2-yl]carbamoyl]-2-methyl-indazol-4-yl]-4-piperidyl]carbamate (Intermediate F46, 35 mg, 50.89 μmol) in DCM (0.5 mL) was added HCl (4.0 M in 1,4-dioxane) (127 μL). The mixture was stirred for 1.5 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 25% to 45% gradient in 7 min; detector, UV 254 nm) to afford 4-[4-(cyclopropylamino)-1-piperidyl]-N-[8-[(difluoromethylsulfonylamino)methyl]-6-methyl-imidazo[1,2-a]pyrazin-2-yl]-2-methyl-indazole-7-carboxamide (Compound 904, 10 mg, 17.02 μmol). LCMS (ES, m/z): 588 [M+H]⁺. ¹H NMR (300 MHz, DMSO-d₆) δ 11.50 (s, 1H), 8.80 (s, 1H), 8.42 (d, J=9.1 Hz, 2H), 8.02 (d, J=8.2 Hz, 1H), 7.06 (t, J=53.1 Hz, 1H), 6.50 (d, J=8.3 Hz, 1H), 4.76 (s, 2H), 4.29 (s, 3H), 3.90 (d, J=12.7 Hz, 2H), 3.08 (t, J=11.7 Hz, 2H), 2.84-2.68 (m, 1H), 2.44 (s, 3H), 2.13 (dd, J=7.0, 3.6 Hz, 1H), 2.00 (d, J=12.6 Hz, 2H), 1.47 (d, J=11.2 Hz, 2H), 0.39 (dt, J=6.1, 3.0 Hz, 2H), 0.24 (q, J=3.4 Hz, 2H).

### Example 297. Synthesis of Compound 909

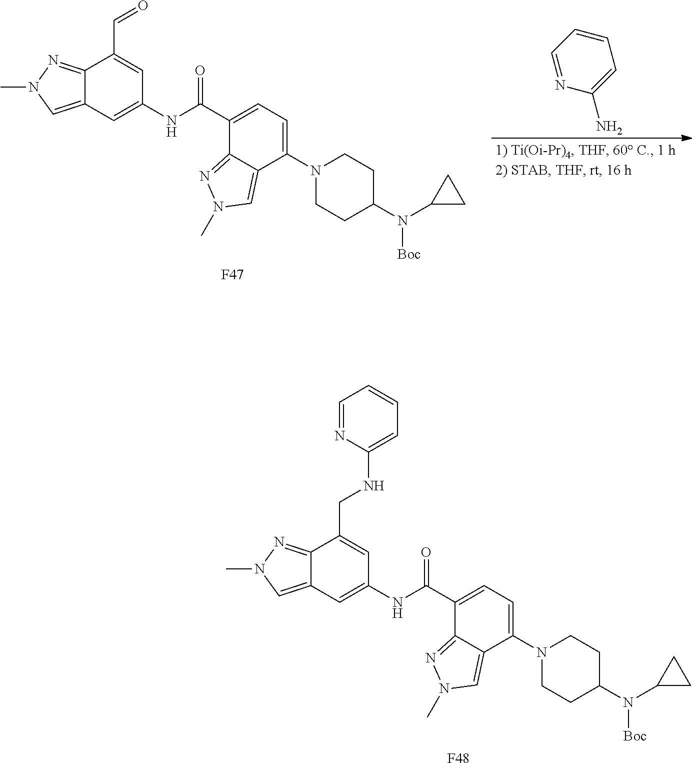

To a solution of tert-butyl N-cyclopropyl-N-[1-[7-[(7-formyl-2-methyl-indazol-5-yl)carbamoyl]-2-methyl-indazol-4-yl]-4-piperidyl]carbamate (Intermediate F47, 70 mg, 122.45 μmol) in THF (1 mL) were added pyridin-2-amine (13.8 mg, 146.94 μmol) and Ti(Oi-Pr)₄ (69.6 mg, 244.90 μmol) at rt. The mixture was stirred for 1 h at 60° C. Then STAB (51.90 mg, 244.90 μmol) was added at rt. The mixture was stirred for 16 h at rt. The reaction was diluted with NaHCO₃ (aq.) (10 mL). The resulting mixture was extracted with DCM (2×10 mL). The combined organic layers were washed with H₂O (10 mL), dried over anhydrous Na₂SO₄. After filtration, the filtrate was concentrated under reduced pressure. The residue was purified by silica gel column chromatography, eluted with DCM/MeOH (19/1) to afford tert-butyl N-cyclopropyl-N-[1-[2-methyl-7-[[2-methyl-7-[(2-pyridylamino)methyl]indazol-5-yl]carbamoyl]indazol-4-yl]-4-piperidyl]carbamate (Intermediate F48, 59 mg, 90.80 μmol). LCMS (ES, m/z): 650 [M+H]⁺.

### Synthesis of Compound 909

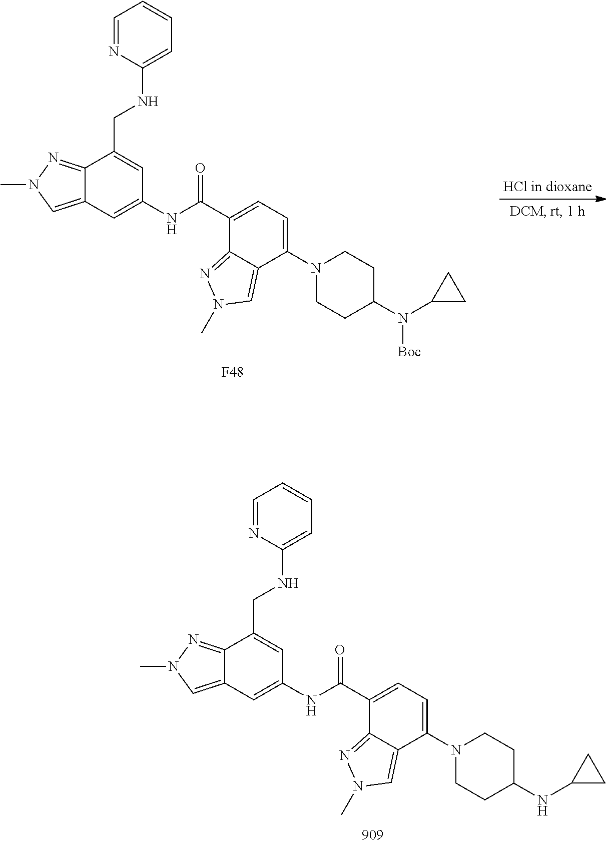

To a solution of tert-butyl N-cyclopropyl-N-[1-[2-methyl-7-[[2-methyl-7-[(2-pyridylamino)methyl]indazol-5-yl]carbamoyl]indazol-4-yl]-4-piperidyl]carbamate (Intermediate F48, 59 mg, 90.80 μmol) in DCM (1 mL) was added HCl (4.0 M in 1,4-dioxane) (227 μL). The mixture was stirred for 1 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 30% to 50% gradient in 7 min; detector, UV 254 nm) to afford 4-[4-(cyclopropylamino)-1-piperidyl]-2-methyl-N-[2-methyl-7-[(2-pyridylamino)methyl]indazol-5-yl]indazole-7-carboxamide (Compound 909, 12 mg, 21.83 μmol). LCMS (ES, m/z): 550 [M+H]⁺. ¹H NMR (400 MHz, DMSO-d₆) δ 11.05 (s, 1H), 8.71 (s, 1H), 8.30-8.24 (m, 2H), 7.99-7.89 (m, 2H), 7.39 (ddd, J=8.7, 7.1, 2.0 Hz, 1H), 7.12 (d, J=1.8 Hz, 1H), 7.05 (t, J=6.1 Hz, 1H), 6.61 (dt, J=8.5, 1.0 Hz, 1H), 6.52-6.41 (m, 2H), 4.82 (d, J=6.0 Hz, 2H), 4.16 (s, 6H), 3.82 (d, J=12.7 Hz, 2H), 3.00 (t, J=11.5 Hz, 2H), 2.74 (s, 1H), 2.21 (s, 1H), 2.10 (dd, J=6.9, 3.5 Hz, 1H), 1.98 (d, J=12.6 Hz, 2H), 1.52-1.39 (m, 2H), 0.37 (dt, J=6.2, 3.0 Hz, 2H), 0.25-0.17 (m, 2H).

### Example 298. Synthesis of Compound 910

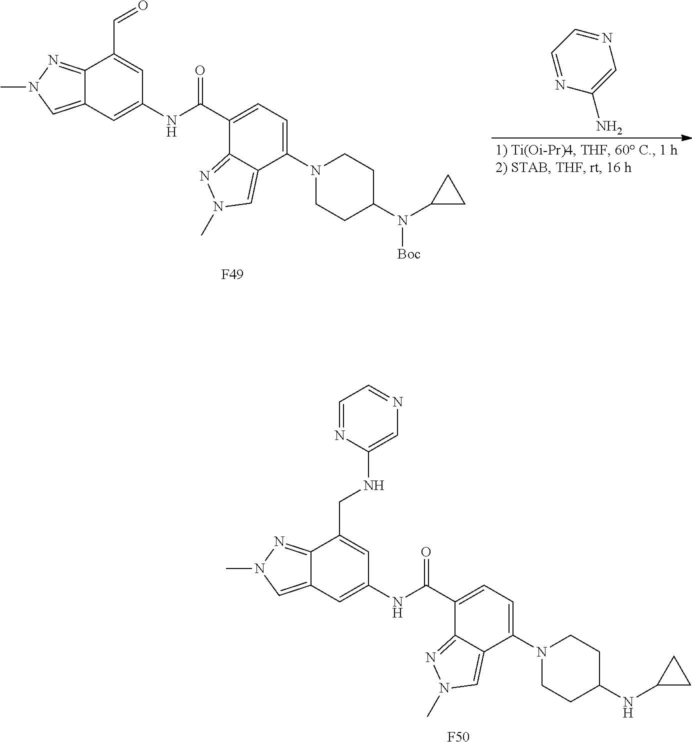

To a solution of tert-butyl N-cyclopropyl-N-[1-[7-[(7-formyl-2-methyl-indazol-5-yl)carbamoyl]-2-methyl-indazol-4-yl]-4-piperidyl]carbamate (Intermediate F49, 70 mg, 122.45 μmol) in THF (1 mL) were added pyrazin-2-amine (13.9 mg, 146.94 μmol) and Ti(Oi-Pr)₄ (69.6 mg, 244.90 μmol) at rt. The mixture was stirred for 1 h at 60° C. Then STAB (51.9 mg, 244.90 μmol) was added at rt. The mixture was stirred for 16 h at rt. The reaction was diluted with NaHCO₃ (aq.) (10 mL). The resulting mixture was extracted with DCM (2×10 mL). The combined organic layers were washed with H₂O (10 mL), dried over anhydrous Na₂SO₄. After filtration, the filtrate was concentrated under reduced pressure. The residue was purified by silica gel column chromatography, eluted with DCM/MeOH (19/1) to afford tert-butyl N-cyclopropyl-N-[1-[2-methyl-7-[[2-methyl-7-[(pyrazin-2-ylamino)methyl]indazol-5-yl]carbamoyl]indazol-4-yl]-4-piperidyl]carbamate (Intermediate F50, 64 mg, 98.34 μmol). LCMS (ES, m/z): 651 [M+H]⁺.

### Synthesis of Compound 910

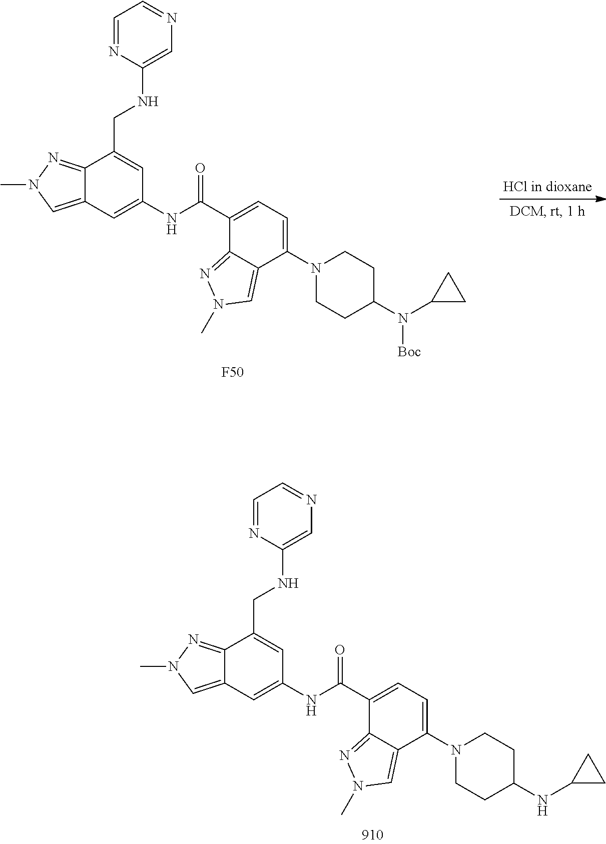

To a solution of tert-butyl N-cyclopropyl-N-[1-[2-methyl-7-[[2-methyl-7-[(pyrazin-2-ylamino)methyl]indazol-5-yl]carbamoyl]indazol-4-yl]-4-piperidyl]carbamate (Intermediate F50, 64 mg, 98.34 μmol) in DCM (1 mL) was added HCl (4.0 M in 1,4-dioxane) (245 μL). The mixture was stirred for 1 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 30% to 50% gradient in 7 min; detector, UV 254 nm) to afford 4-[4-(cyclopropylamino)-1-piperidyl]-2-methyl-N-[2-methyl-7-[(pyrazin-2-ylamino)methyl]indazol-5-yl]indazole-7-carboxamide (Compound 910, 22 mg, 39.95 μmol). LCMS (ES, m/z): 551 [M+H]⁺. ¹H NMR (400 MHz, DMSO-d₆) δ 11.07 (s, 1H), 8.71 (s, 1H), 8.29 (s, 1H), 8.26 (d, J=1.8 Hz, 1H), 8.09 (d, J=1.5 Hz, 1H), 7.96-7.90 (m, 2H), 7.67 (d, J=2.8 Hz, 1H), 7.63 (t, J=5.9 Hz, 1H), 7.16 (d, J=1.8 Hz, 1H), 6.44 (d, J=8.2 Hz, 1H), 4.83 (d, J=5.8 Hz, 2H), 4.17 (d, J=5.5 Hz, 6H), 3.82 (d, J=12.4 Hz, 2H), 3.00 (t, J=11.7 Hz, 2H), 2.74 (s, 1H), 2.21 (s, 1H), 2.10 (tt, J=6.8, 3.6 Hz, 1H), 1.98 (d, J=12.5 Hz, 2H), 1.45 (d, J=11.1 Hz, 2H), 0.38 (td, J=6.3, 4.1 Hz, 2H), 0.25-0.17 (m, 2H).

### Example 299. Synthesis of Compound 911

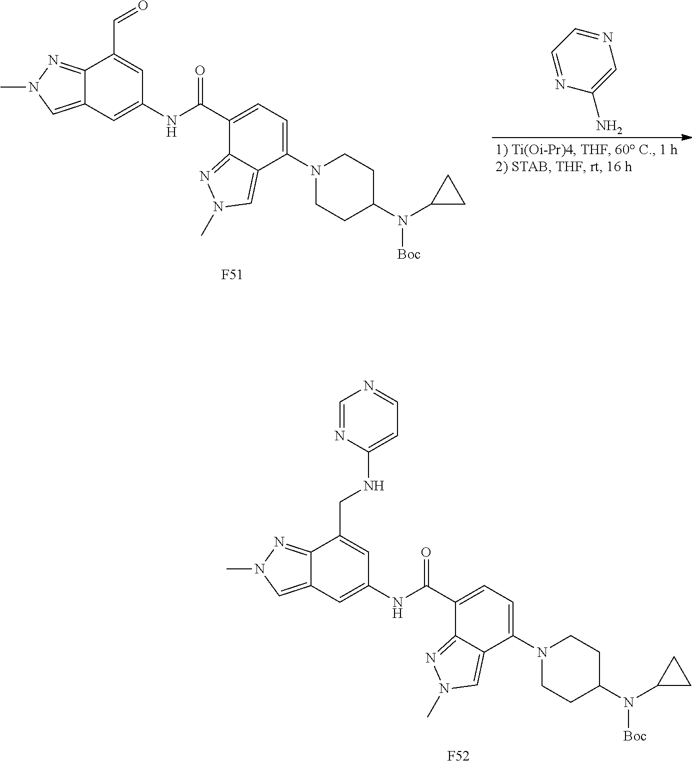

To a solution of tert-butyl N-cyclopropyl-N-[1-[7-[(7-formyl-2-methyl-indazol-5-yl)carbamoyl]-2-methyl-indazol-4-yl]-4-piperidyl]carbamate (Intermediate F51, 70 mg, 122.45 μmol) in THF (1 mL) were added pyrimidin-4-amine (13.9 mg, 146.94 μmol) and Ti(Oi-Pr)₄ (69.6 mg, 244.90 μmol) at rt. The mixture was stirred for 1 h at 60° C. Then STAB (51.9 mg, 244.90 μmol) was added at rt. The mixture was stirred for 16 h at rt. The reaction was diluted with NaHCO₃ (aq.) (10 mL). The resulting mixture was extracted with DCM (2×10 mL). The combined organic layers were washed with H₂O (10 mL), dried over anhydrous Na₂SO₄. After filtration, the filtrate was concentrated under reduced pressure. The residue was purified by Prep-TLC (DCM/MeOH=10/1) to afford tert-butyl N-cyclopropyl-N-[1-[2-methyl-7-[[2-methyl-7-[(pyrimidin-4-ylamino)methyl]indazol-5-yl]carbamoyl]indazol-4-yl]-4-piperidyl]carbamate (Intermediate F52, 25 mg, 38.42 μmol, 31.37% yield) as a yellow solid. LCMS (ES, m/z): 651 [M+H]⁺.

### Synthesis of Compound 911

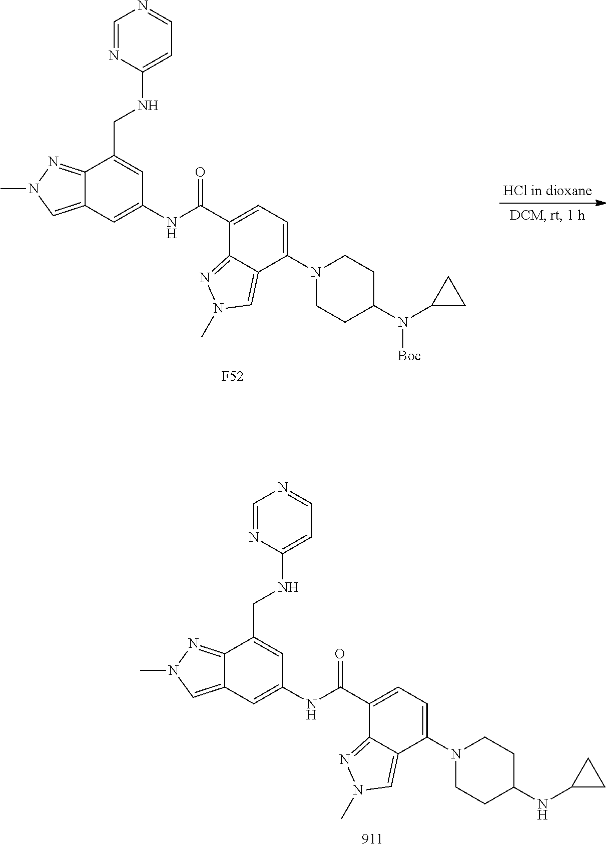

To a solution of tert-butyl N-cyclopropyl-N-[1-[2-methyl-7-[[2-methyl-7-[(pyrimidin-4-ylamino)methyl]indazol-5-yl]carbamoyl]indazol-4-yl]-4-piperidyl]carbamate (Intermediate F52, 25 mg, 38.42 μmol) in DCM (0.5 mL) was added HCl (4.0 M in 1,4-dioxane) (96 μL). The mixture was stirred for 1 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 30% to 50% gradient in 7 min; detector, UV 254 nm) to afford 4-[4-(cyclopropylamino)-1-piperidyl]-2-methyl-N-[2-methyl-7-[(pyrimidin-4-ylamino)methyl]indazol-5-yl]indazole-7-carboxamide (Compound 911, 5 mg, 9.08 μmol). LCMS (ES, m/z): 551 [M+H]⁺. ¹H NMR (400 MHz, DMSO-d₆) δ 11.08 (s, 1H), 8.71 (s, 1H), 8.42 (s, 1H), 8.30 (s, 1H), 8.26 (d, J=1.8 Hz, 1H), 8.07 (d, J=6.0 Hz, 1H), 7.96-7.90 (m, 2H), 7.16-7.11 (m, 1H), 6.62 (s, 1H), 6.45 (d, J=8.2 Hz, 1H), 4.85 (s, 2H), 4.17 (d, J=3.9 Hz, 6H), 3.86-3.79 (m, 2H), 3.01 (t, J=11.2 Hz, 2H), 2.75 (s, 1H), 2.21 (s, 1H), 2.10 (dd, J=7.1, 3.5 Hz, 1H), 1.98 (d, J=12.6 Hz, 2H), 1.46 (q, J=9.7 Hz, 2H), 0.38 (td, J=6.3, 4.0 Hz, 2H), 0.25-0.17 (m, 2H).

### Example 300. Synthesis of Compound 755

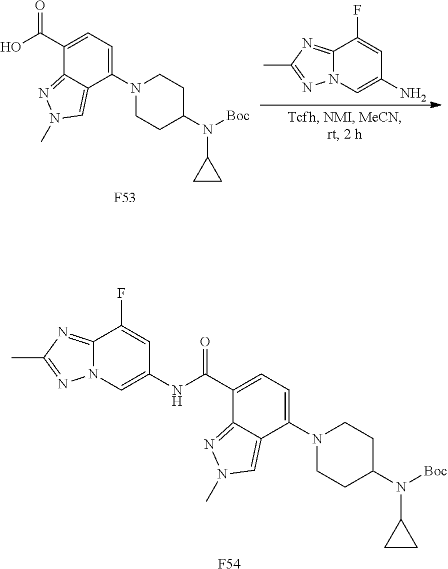 [SMILES: Cn1cc2c(N3CCC(N(C(=O)OC(C)(C)C)C4CC4)CC3)ccc(C(=O)O)c2n1 ; Cc1nc2c(F)cc(N)cn2n1 ; Cc1nc2c(F)cc(NC(=O)c3ccc(N4CCC(N(C(=O)OC(C)(C)C)C5CC5)CC4)c4cn(C)nc34)cn2n1 ; "F53" ; "F54"]

To a stirred mixture of 4-[4-[tert-butoxycarbonyl(cyclopropyl)amino]-1-piperidyl]-2-methyl-indazole-7-carboxylic acid (Intermediate F53, 0.07 g, 168.88 μmol) and 8-fluoro-2-methyl-[1,2,4]triazolo[1,5-a]pyridin-6-amine (33.7 mg, 202.66 μmol) in MeCN (2 mL) were added NMI (41.6 mg, 506.64 μmol), TCFH (71.8 mg, 253.32 μmol) at room temperature. The resulting mixture was stirred for 2 h at room temperature. The resulting mixture was diluted with water (3 mL). The resulting mixture was extracted with EA (3×3 mL). The combined organic layers were washed with water (1×8 mL), brine (1×8 mL), dried over anhydrous Na₂SO₄. After filtration, the filtrate was concentrated under reduced pressure. The mixture was purified by silica gel column chromatography, eluted with PE/EA (1:9) to afford tert-butyl N-cyclopropyl-N-[1-[7-[(8-fluoro-2-methyl-[1,2,4]triazolo[1,5-a]pyridin-6-yl)carbamoyl]-2-methyl-indazol-4-yl]-4-piperidyl]carbamate (Intermediate F54, 0.085 g, 151.07 μmol). LCMS (ES, m/z): 563 [M+H]⁺.

### Synthesis of Compound 755

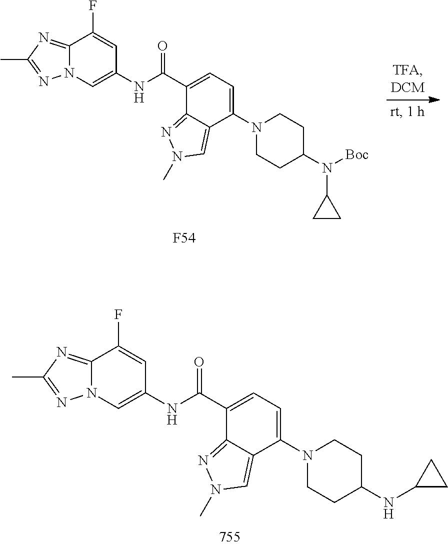 [SMILES: Cc1nc2c(F)cc(NC(=O)c3ccc(N4CCC(N(C(=O)OC(C)(C)C)C5CC5)CC4)c4cn(C)nc34)cn2n1 ; Cc1nc2c(F)cc(NC(=O)c3ccc(N4CCC(NC5CC5)CC4)c4cn(C)nc34)cn2n1 ; "F54"]

To a solution of tert-butyl N-cyclopropyl-N-[1-[7-[(8-fluoro-2-methyl-[1,2,4]triazolo[1,5-a]pyridin-6-yl)carbamoyl]-2-methyl-indazol-4-yl]-4-piperidyl]carbamate (Intermediate F54, 0.085 g, 151.07 μmol) in DCM (1 mL) was added TFA (0.4 mL). The reaction was stirred for 1 h at rt. The resulting mixture was concentrated under reduced pressure and adjusted to pH 8 with saturated NaHCO₃ (aq.). After filtration, the filtrate was concentrated under reduced pressure. The residue was purified by reversed-phase flash chromatography with the following conditions (column, C18 silica gel; mobile phase, CH₃CN in water (0.1% NH₃·H₂O), 40% to 70% gradient in 10 min; detector, UV 254 nm) to afford 4-[4-(cyclopropylamino)-1-piperidyl]-N-(8-fluoro-2-methyl-[1,2,4]triazolo[1,5-a]pyridin-6-yl)-2-methyl-indazole-7-carboxamide (Compound 755, 0.01 g, 21.62 μmol). LCMS (ES, m/z): 463 [M+H]⁺. ¹H NMR (400 MHz, DMSO-d₆) δ 11.28 (s, 1H), 9.50 (d, J=1.8 Hz, 1H), 8.78 (s, 1H), 7.97 (d, J=8.1 Hz, 1H), 7.88 (dd, J=11.7, 1.7 Hz, 1H), 6.48 (d, J=8.2 Hz, 1H), 4.30 (s, 3H), 3.91 (dd, J=10.6, 6.2 Hz, 2H), 3.14-3.03 (m, 2H), 2.78 (s, 1H), 2.25 (s, 1H), 2.12 (dq, J=6.7, 3.5 Hz, 1H), 2.04-1.96 (m, 2H), 1.48-1.44 (m, 2H), 0.40 (td, J=6.3, 4.1 Hz, 2H), 0.23 (p, J=4.1 Hz, 2H).

### Example 301. Synthesis of Compound 756

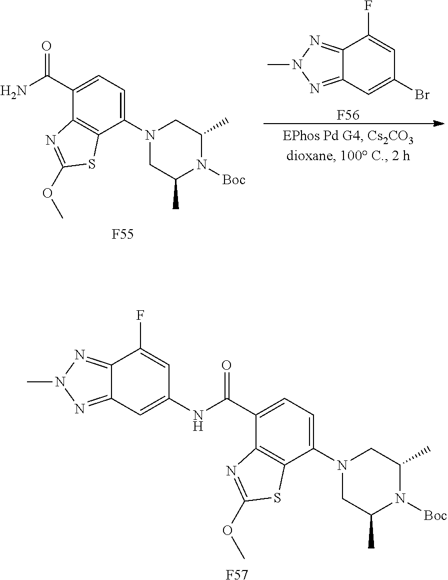

To a stirred mixture of tert-butyl (2S,6S)-4-(4-carbamoyl-2-methoxy-1,3-benzothiazol-7-yl)-2,6-dimethyl-piperazine-1-carboxylate (Intermediate F55, 50 mg, 118.90 μmol) and 6-bromo-4-fluoro-2-methyl-benzotriazole (Intermediate F56, 32.8 mg, 142.68 μmol) in Dioxane (1 mL) were added Cs₂CO₃ (77.4 mg, 237.80 μmol) and EPhos Pd G4 (10.9 mg, 11.89 μmol) at room temperature under nitrogen atmosphere. The resulting mixture was stirred for 2 h at 100° C. under nitrogen atmosphere. The resulting mixture was concentrated under reduced pressure. The residue was purified by silica gel column chromatography, eluted with PE/EA (1:3) to afford tert-butyl (2S,6S)-4-[4-[(7-fluoro-2-methyl-benzotriazol-5-yl)carbamoyl]-2-methoxy-1,3-benzothiazol-7-yl]-2,6-dimethyl-piperazine-1-carboxylate (Intermediate F57, 55 mg, 96.55 μmol). LCMS (ES, m/z): 570 [M+H]⁺.

### Synthesis of Compound 756

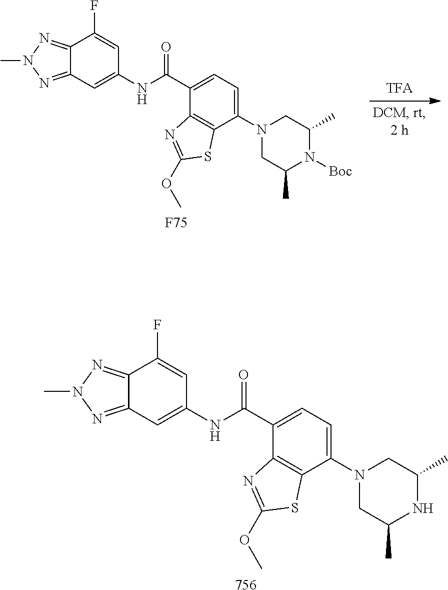

To a stirred solution of tert-butyl (2S,6S)-4-[4-[(7-fluoro-2-methyl-benzotriazol-5-yl)carbamoyl]-2-methoxy-1,3-benzothiazol-7-yl]-2,6-dimethyl-piperazine-1-carboxylate (Intermediate F57, 55 mg, 96.55 μmol) in DCM (2 mL) was added TFA (1 mL) at rt. The resulting mixture was stirred for 2 h. The resulting mixture was concentrated under reduced pressure. The crude product was purified by Prep-HPLC with the following conditions (SunFire Prep C18 OBD Column 19*150 mm, 5 Um 10 nm, mobile phase, MeCN in water (0.05% TFA), 30% to 40% gradient in 7 min; detector, UV 254 nm) to afford 7-[(3S,5S)-3,5-dimethylpiperazin-1-yl]-N-(7-fluoro-2-methyl-benzotriazol-5-yl)-2-methoxy-1,3-benzothiazole-4-carboxamide (Compound 756, 16 mg, 34.08 μmol). LCMS (ES, m/z): 470 [M+H]⁺. ¹H NMR (400 MHz, DMSO-d₆) δ 11.56 (s, 1H), 8.93 (s, 2H), 8.35 (s, 1H), 8.18 (d, J=8.5 Hz, 1H), 7.55 (d, J=12.1 Hz, 1H), 7.21 (d, J=8.5 Hz, 1H), 4.52 (s, 3H), 4.45 (s, 3H), 3.78 (s, 2H), 3.40-3.35 (m, 2H), 3.30-3.24 (m, 2H), 1.42 (d, J=6.5 Hz, 6H).

### Example 302. Synthesis of Compound 757

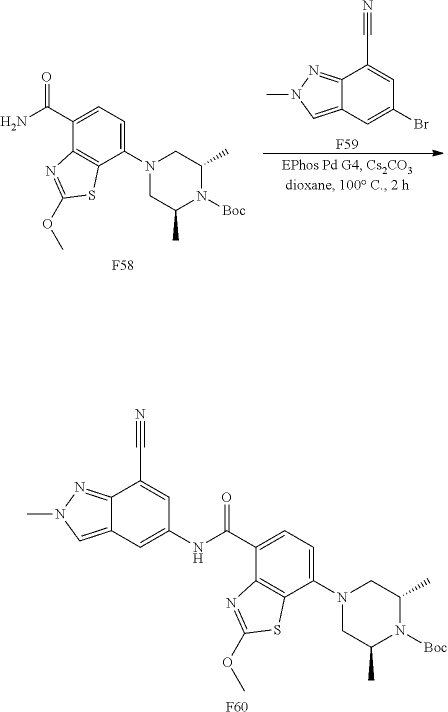

To a stirred mixture of tert-butyl (2S,6S)-4-(4-carbamoyl-2-methoxy-1,3-benzothiazol-7-yl)-2,6-dimethyl-piperazine-1-carboxylate (Intermediate F58, 50 mg, 118.90 μmol) and 5-bromo-2-methyl-indazole-7-carbonitrile (Intermediate F59, 33.6 mg, 142.68 μmol) in Dioxane (1 mL) were added Cs₂CO₃ (18.5 mg, 237.80 μmol) and EPhos Pd G4 (10.9 mg, 11.89 μmol) at room temperature under nitrogen atmosphere. The resulting mixture was stirred for 2 h at 100° C. under nitrogen atmosphere. The resulting mixture was concentrated under reduced pressure. The residue was purified by silica gel column chromatography, eluted with PE/EA (1:3) to afford tert-butyl (2S,6S)-4-[4-[(7-cyano-2-methyl-indazol-5-yl)carbamoyl]-2-methoxy-1,3-benzothiazol-7-yl]-2,6-dimethyl-piperazine-1-carboxylate (Intermediate F60, 45 mg, 78.17 μmol). LCMS (ES, m/z): 576 [M+H]⁺.

### Synthesis of Compound 757

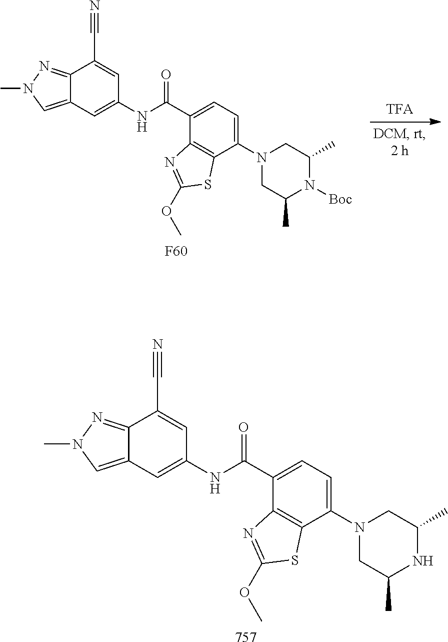

To a stirred solution of tert-butyl (2S,6S)-4-[4-[(7-cyano-2-methyl-indazol-5-yl)carbamoyl]-2-methoxy-1,3-benzothiazol-7-yl]-2,6-dimethyl-piperazine-1-carboxylate (Intermediate F60, 40 mg, 69.48 μmol) in DCM (2 mL) was added TFA (1 mL) at rt. The resulting mixture was stirred for 2 h. The resulting mixture was concentrated under reduced pressure. The crude product was purified by Prep-HPLC with the following conditions (SunFire Prep C18 OBD Column 19*150 mm, 5 Um 10 nm, mobile phase, MeCN in water (0.05% NH₄OH), 50% to 60% gradient in 7 min; detector, UV 254 nm) to afford N-(7-cyano-2-methyl-indazol-5-yl)-7-[(3S,5S)-3,5-dimethylpiperazin-1-yl]-2-methoxy-1,3-benzothiazole-4-carboxamide (Compound 757, 8.7 mg, 18.29 μmol). LCMS (ES, m/z): 476 [M+H]⁺. ¹H NMR (400 MHz, DMSO-d₆) δ 11.46 (s, 1H), 8.63-8.56 (m, 2H), 8.13-8.10 (m, 2H), 7.06 (d, J=8.6 Hz, 1H), 4.42 (s, 3H), 4.25 (s, 3H), 3.24 (tt, J=9.4, 4.6 Hz, 2H), 3.12 (dd, J=11.5, 3.1 Hz, 2H), 2.93 (dd, J=11.5, 6.1 Hz, 2H), 1.18 (d, J=6.4 Hz, 6H).

### Example 303. Synthesis of Compound 758

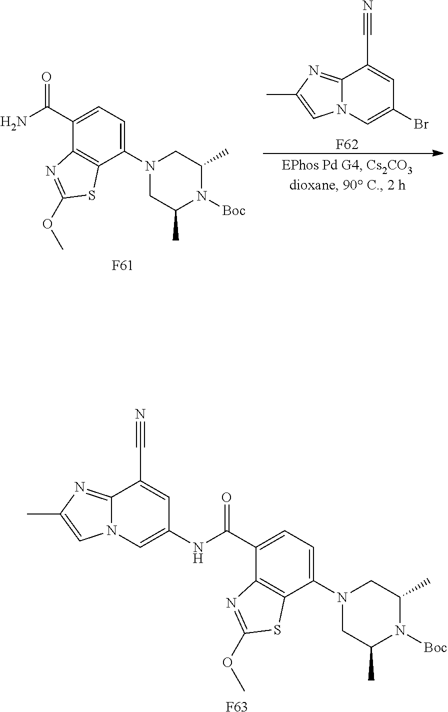

To a stirred mixture of tert-butyl (2S,6S)-4-(4-carbamoyl-2-methoxy-1,3-benzothiazol-7-yl)-2,6-dimethyl-piperazine-1-carboxylate (Intermediate F61, 50 mg, 118.90 μmol) and 6-bromo-2-methyl-imidazo[1,2-a]pyridine-8-carbonitrile (Intermediate F62, 33.6 mg, 142.68 μmol) in dioxane (1 mL) were added Cs₂CO₃ (77.4 mg, 237.80 μmol) and EPhos Pd G4 (10.9 mg, 11.89 μmol) at room temperature under nitrogen atmosphere. The resulting mixture was stirred for 2 h at 100° C. under nitrogen atmosphere. The resulting mixture was concentrated under reduced pressure. The residue was purified by silica gel column chromatography, eluted with PE/EA (1:3) to afford tert-butyl (2S,6S)-4-[4-[(8-cyano-2-methyl-imidazo[1,2-a]pyridin-6-yl)carbamoyl]-2-methoxy-1,3-benzothiazol-7-yl]-2,6-dimethyl-piperazine-1-carboxylate (Intermediate F63, 60 mg, 104.22 μmol). LCMS (ES, m/z): 576 [M+H]⁺.

### Synthesis of Compound 758

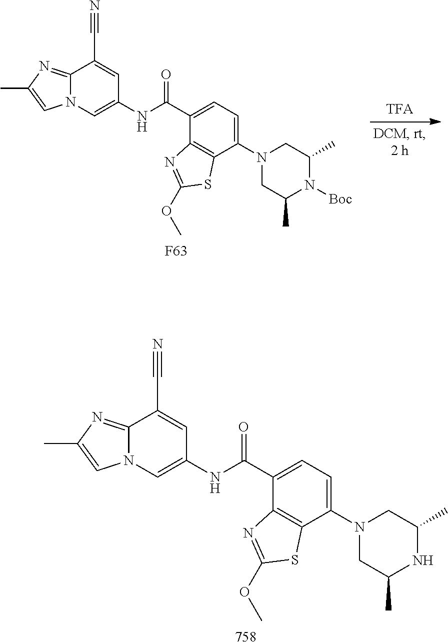

To a stirred solution of tert-butyl (2S,6S)-4-[4-[(8-cyano-2-methyl-imidazo[1,2-a]pyridin-6-yl)carbamoyl]-2-methoxy-1,3-benzothiazol-7-yl]-2,6-dimethyl-piperazine-1-carboxylate (Intermediate F63, 60 mg, 104.22 μmol) in DCM (2 mL) was added TFA (1.20 mL) at rt. The resulting mixture was stirred for 2 h. The resulting mixture was concentrated under reduced pressure. The crude product was purified by Prep-HPLC with the following conditions (SunFire Prep C18 OBD Column 19*150 mm, 5 Um 10 nm, mobile phase, MeCN in water (0.05% NH₄OH), 50% to 60% gradient in 7 min; detector, UV 254 nm) to afford N-(8-cyano-2-methyl-imidazo[1,2-a]pyridin-6-yl)-7-[(3S,5S)-3,5-dimethylpiperazin-1-yl]-2-methoxy-1,3-benzothiazole-4-carboxamide (Compound 758, 25.7 mg, 54.04 μmol). LCMS (ES, m/z): 476 [M+H]⁺. ¹H NMR (400 MHz, DMSO-d₆) δ 11.32 (s, 1H), 9.59 (d, J=2.0 Hz, 1H), 8.10 (d, J=8.4 Hz, 1H), 7.97 (s, 1H), 7.93 (s, 1H), 7.05 (d, J=8.5 Hz, 1H), 4.42 (s, 3H), 3.23 (tt, J=9.6, 4.6 Hz, 2H), 3.12 (dd, J=11.5, 3.1 Hz, 2H), 2.93 (dd, J=11.5, 6.1 Hz, 2H), 2.39 (s, 3H), 1.17 (d, J=6.4 Hz, 6H).

### Example 304. Synthesis of Compound 759

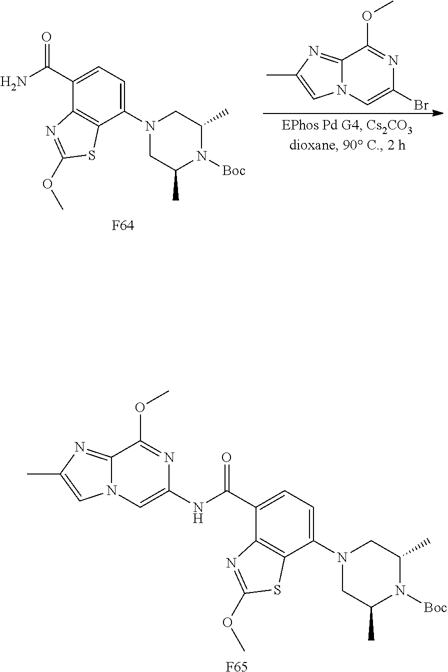

To a stirred mixture of tert-butyl (2S,6S)-4-(4-carbamoyl-2-methoxy-1,3-benzothiazol-7-yl)-2,6-dimethyl-piperazine-1-carboxylate (Intermediate F64, 0.055 g, 130.79 μmol) and 6-bromo-8-methoxy-2-methyl-imidazo[1,2-a]pyrazine (37.9 mg, 156.95 μmol) in Dioxane (1 mL) were added Cs₂CO₃ (85.3 mg, 261.58 μmol), EPhos Pd G4 (12.1 mg, 13.08 μmol) at room temperature under nitrogen atmosphere. The resulting mixture was stirred for 2 h at 90° C. under nitrogen atmosphere. The mixture was allowed to cool down to room temperature. After filtration, the filtrate was concentrated under reduced pressure. The mixture was purified by silica gel column chromatography, eluted with PE/EA (1:5) to afford tert-butyl (2S,6S)-4-[2-methoxy-4-[(8-methoxy-2-methyl-imidazo[1,2-a]pyrazin-6-yl)carbamoyl]-1,3-benzothiazol-7-yl]-2,6-dimethyl-piperazine-1-carboxylate (Intermediate F65, 0.058 g, 99.71 μmol). LCMS (ES, m/z): 582 [M+H]⁺.

### Synthesis of Compound 759

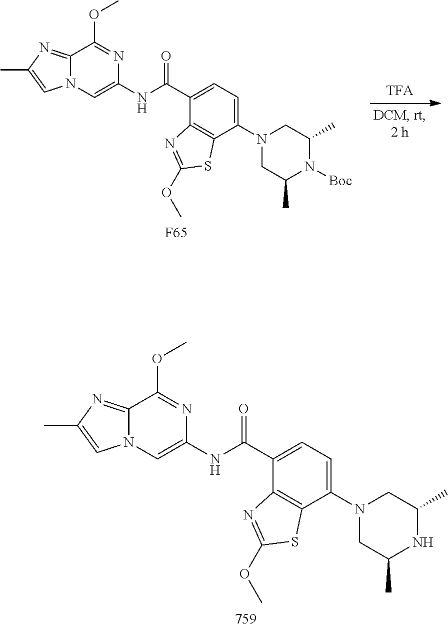

To a solution of tert-butyl (2S,6S)-4-[2-methoxy-4-[(8-methoxy-2-methyl-imidazo[1,2-a]pyrazin-6-yl)carbamoyl]-1,3-benzothiazol-7-yl]-2,6-dimethyl-piperazine-1-carboxylate (Intermediate F65, 0.06 g, 103.15 μmol) in DCM (1 mL) was added TFA (0.4 mL). The reaction was stirred for 2 h at rt. The resulting mixture was concentrated under reduced pressure and adjusted to pH 8 with saturated NaHCO₃ (aq.). After filtration, the filtrate was concentrated under reduced pressure. The residue was purified by reversed-phase flash chromatography with the following conditions (SunFire Prep C18 OBD Column 19*150 mm, 5 μm 10 nm, mobile phase, MeCN in water (0.01% TFA, 10% to 30% gradient in 7 min; detector, UV 254 nm) to afford 7-((3S,5S)-3,5-dimethylpiperazin-1-yl)-2-methoxy-N-(8-methoxy-2-methylimidazo[1,2-a]pyrazin-6-yl)benzo[d]thiazole-4-carboxamide 2,2,2-trifluoroacetate (Compound 759, 0.02 g, 33.58 μmol). LCMS (ES, m/z): 482 [M+H]⁺. ¹H NMR (300 MHz, DMSO-d₆) δ 11.94 (s, 1H), 8.97 (s, 1H), 8.92 (s, 2H), 8.17 (d, J=8.5 Hz, 1H), 7.97 (s, 1H), 7.16 (d, J=8.6 Hz, 1H), 4.41 (s, 3H), 3.97 (s, 3H), 3.80-3.67 (m, 2H), 3.40-3.30 (m, 2H), 3.20 (dd, J=13.1, 6.3 Hz, 2H), 2.34 (s, 3H), 1.39 (d, J=6.5 Hz, 6H).

### Example 305. Synthesis of Compound 761

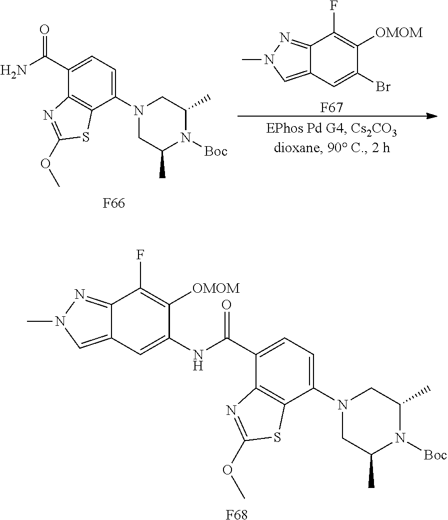

To a stirred mixture of tert-butyl (2S,6S)-4-(4-carbamoyl-2-methoxy-1,3-benzothiazol-7-yl)-2,6-dimethyl-piperazine-1-carboxylate (Intermediate F66, 50 mg, 118.90 μmol) and 5-bromo-7-fluoro-6-(methoxymethoxy)-2-methyl-indazole (Intermediate F57, 41.2 mg, 142.68 μmol) in dioxane (1 mL) were added Cs₂CO₃ (77.4 mg, 237.80 μmol) and EPhos Pd G4 (10.9 mg, 11.89 μmol) at room temperature under nitrogen atmosphere. The resulting mixture was stirred for 2 h at 90° C. under nitrogen atmosphere. The resulting mixture was concentrated under reduced pressure. The residue was purified by silica gel column chromatography, eluted with PE/EA (1:3) to afford tert-butyl (2S,6S)-4-[4-[[7-fluoro-6-(methoxymethoxy)-2-methyl-indazol-5-yl]carbamoyl]-2-methoxy-1,3-benzothiazol-7-yl]-2,6-dimethyl-piperazine-1-carboxylate (Intermediate F68, 55 mg, 87.48 μmol). LCMS (ES, m/z): 629 [M+H]⁺.

### Synthesis of Compound 761

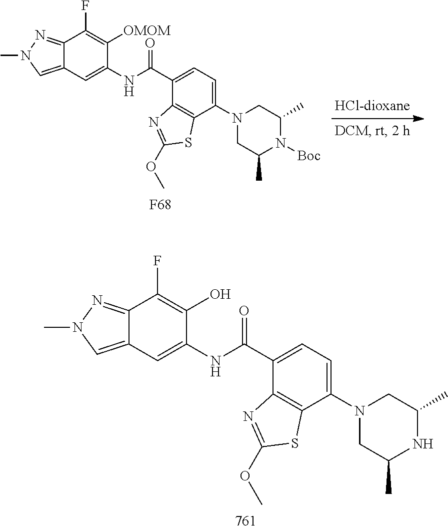

To a stirred solution of tert-butyl (2S,6S)-4-[4-[[7-fluoro-6-(methoxymethoxy)-2-methyl-indazol-5-yl]carbamoyl]-2-methoxy-1,3-benzothiazol-7-yl]-2,6-dimethyl-piperazine-1-carboxylate (Intermediate F68, 55 mg, 87.48 μmol) in DCM (2 mL) was added HCl-dioxane (1 mL) at rt. The resulting mixture was stirred for 2 h. The resulting mixture was concentrated under reduced pressure. The crude product was purified by Prep-HPLC with the following conditions (SunFire Prep C18 OBD Column 19*150 mm, 5 Um 10 nm, mobile phase, MeCN in water (0.05% NH₄OH), 30% to 40% gradient in 7 min; detector, UV 254 nm) to afford 7-[(3S,5S)-3,5-dimethylpiperazin-1-yl]-N-(7-fluoro-6-hydroxy-2-methyl-indazol-5-yl)-2-methoxy-1,3-benzothiazole-4-carboxamide (Compound 761, 19.5 mg, 40.24 μmol). LCMS (ES, m/z): 485 [M+H]⁺. ¹H NMR (400 MHz, DMSO-d₆) δ 11.80 (s, 1H), 8.67 (s, 1H), 8.32 (d, J=2.7 Hz, 1H), 8.27-8.18 (m, 1H), 7.07 (d, J=8.6 Hz, 1H), 4.40 (s, 3H), 4.11 (s, 3H), 3.30-3.20 (m, 2H), 3.12 (dd, J=11.6, 3.1 Hz, 2H), 2.94 (dd, J=11.6, 6.1 Hz, 2H), 1.19 (d, J=6.4 Hz, 6H).

### Example 306. Synthesis of Compound 762

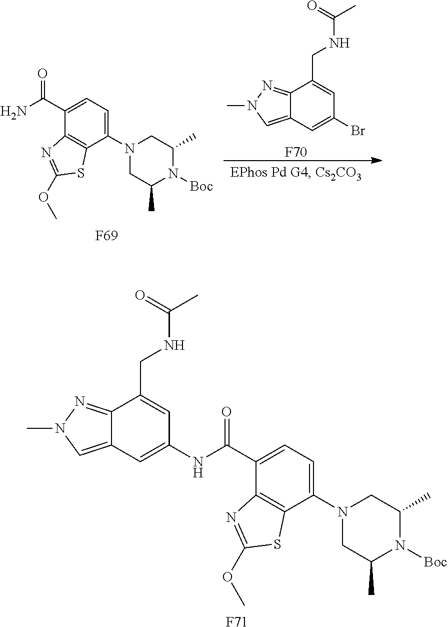

To a stirred mixture of tert-butyl (2S,6S)-4-(4-carbamoyl-2-methoxy-1,3-benzothiazol-7-yl)-2,6-dimethyl-piperazine-1-carboxylate (Intermediate F69, 50 mg, 118.90 μmol) and N-[(5-bromo-2-methyl-indazol-7-yl)methyl]acetamide (Intermediate F70, 40.2 mg, 142.68 μmol) in Dioxane (1 mL) were added Cs₂CO₃ (18.5 mg, 237.80 μmol) and EPhos Pd G4 (10.9 mg, 11.89 μmol) at room temperature under nitrogen atmosphere. The resulting mixture was stirred for 2 h at 90° C. under nitrogen atmosphere. The resulting mixture was concentrated under reduced pressure. The residue was purified by silica gel column chromatography, eluted with PE/EA (1:3) to afford tert-butyl (2S,6S)-4-[4-[[7-(acetamidomethyl)-2-methyl-indazol-5-yl]carbamoyl]-2-methoxy-1,3-benzothiazol-7-yl]-2,6-dimethyl-piperazine-1-carboxylate (Intermediate F71, 60 mg, 96.50 μmol). LCMS (ES, m/z): 622 [M+H]⁺.

### Synthesis of Compound 762

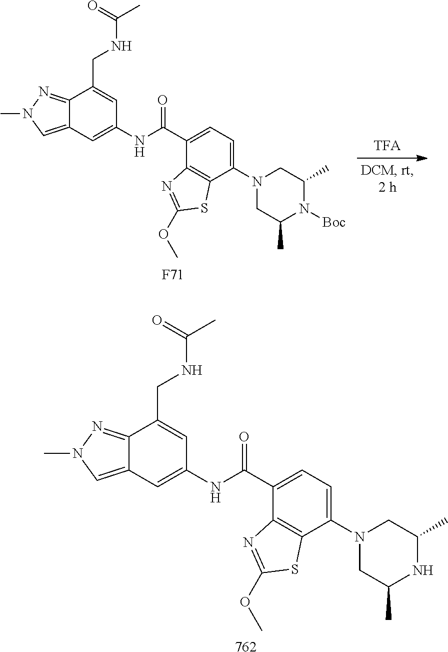

To a stirred solution of tert-butyl (2S,6S)-4-[4-[[7-(acetamidomethyl)-2-methyl-indazol-5-yl]carbamoyl]-2-methoxy-1,3-benzothiazol-7-yl]-2,6-dimethyl-piperazine-1-carboxylate (Intermediate F71, 60 mg, 96.50 μmol) in DCM (2 mL) was added TFA (1 mL) at rt. The resulting mixture was stirred for 2 h. The resulting mixture was concentrated under reduced pressure. The crude product was purified by Prep-HPLC with the following conditions (SunFire Prep C18 OBD Column 19*150 mm, 5 Um 10 nm, mobile phase, MeCN in water (0.05% TFA), 30% to 40% gradient in 7 min; detector, UV 254 nm) to afford N-[7-(acetamidomethyl)-2-methyl-indazol-5-yl]-7-[(3S,5S)-3,5-dimethylpiperazin-1-yl]-2-methoxy-1,3-benzothiazole-4-carboxamide (Compound 762, 14 mg, 26.84 μmol). LCMS (ES, m/z): 522 [M+H]⁺. ¹H NMR (400 MHz, DMSO-d₆) δ 11.52 (s, 1H), 8.45 (t, J=5.9 Hz, 1H), 8.40 (d, J=1.8 Hz, 1H), 8.32 (s, 1H), 8.15 (d, J=8.5 Hz, 1H), 7.10-7.03 (m, 2H), 4.61 (d, J=5.9 Hz, 2H), 4.43 (s, 3H), 4.17 (s, 3H), 3.24 (td, J=6.3, 3.0 Hz, 2H), 3.11 (dd, J=11.4, 3.1 Hz, 2H), 2.92 (dd, J=11.5, 6.1 Hz, 2H), 1.93 (s, 3H), 1.18 (d, J=6.4 Hz, 6H).

### Example 307. Synthesis of Compound 763

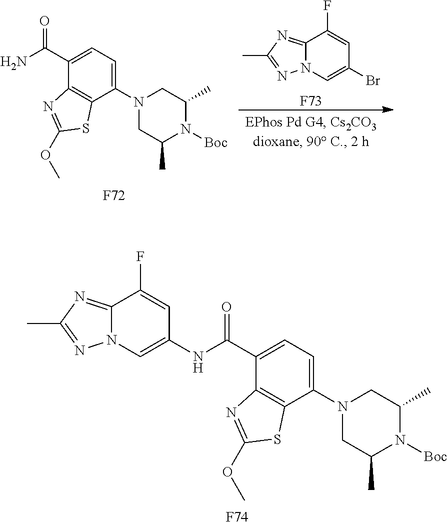

To a stirred mixture of tert-butyl (2S,6S)-4-(4-carbamoyl-2-methoxy-1,3-benzothiazol-7-yl)-2,6-dimethyl-piperazine-1-carboxylate (Intermediate F72, 40 mg, 95.12 μmol) and 6-bromo-8-fluoro-2-methyl-[1,2,4]triazolo[1,5-a]pyridine (Intermediate F73, 26.2 mg, 114.14 μmol) in dioxane (1 mL) were added Cs₂CO₃ (61.9 mg, 190.24 μmol) and EPhos Pd G4 (8.7 mg, 9.51 μmol) at room temperature under nitrogen atmosphere. The resulting mixture was stirred for 2 h at 90° C. under nitrogen atmosphere. The resulting mixture was concentrated under reduced pressure. The residue was purified by silica gel column chromatography, eluted with PE/EA (1:3) to afford tert-butyl (2S,6S)-4-[4-[(8-fluoro-2-methyl-[1,2,4]triazolo[1,5-a]pyridin-6-yl)carbamoyl]-2-methoxy-1,3-benzothiazol-7-yl]-2,6-dimethyl-piperazine-1-carboxylate (Intermediate F74, 45 mg, 79.00 μmol). LCMS (ES, m/z): 570 [M+H]⁺.

### Synthesis of Compound 763

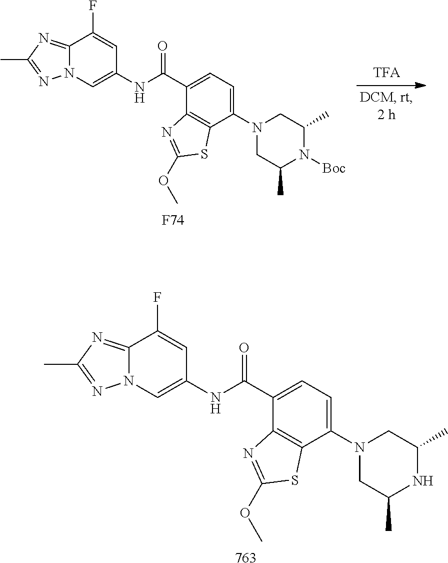

To a stirred solution of tert-butyl (2S,6S)-4-[4-[(8-fluoro-2-methyl-[1,2,4]triazolo[1,5-a]pyridin-6-yl)carbamoyl]-2-methoxy-1,3-benzothiazol-7-yl]-2,6-dimethyl-piperazine-1-carboxylate (Intermediate F74, 45 mg, 79.00 μmol) in DCM (2 mL) was added TFA (1 mL) at rt. The resulting mixture was stirred for 2 h. The resulting mixture was concentrated under reduced pressure. The crude product was purified by Prep-HPLC with the following conditions (SunFire Prep C18 OBD Column 19*150 mm, 5 Um 10 nm, mobile phase, MeCN in water (0.05% NH₄OH), 50% to 60% gradient in 7 min; detector, UV 254 nm) to afford 7-[(3S,5S)-3,5-dimethylpiperazin-1-yl]-N-(8-fluoro-2-methyl-[1,2,4]triazolo[1,5-a]pyridin-6-yl)-2-methoxy-1,3-benzothiazole-4-carboxamide (Compound 763, 10.1 mg, 21.51 μmol). LCMS (ES, m/z): 470 [M+H]⁺. ¹H NMR (400 MHz, DMSO-d₆) δ 11.38 (s, 1H), 9.44 (s, 1H), 8.92 (s, 2H), 8.13 (d, J=8.4 Hz, 1H), 7.75 (d, J=11.4 Hz, 1H), 7.18 (d, J=8.6 Hz, 1H), 4.41 (s, 3H), 3.76 (s, 2H), 3.36 (d, J=12.8 Hz, 2H), 3.23 (dd, J=13.3, 6.3 Hz, 2H), 2.50 (s, 3H), 1.39 (d, J=6.5 Hz, 6H).

### Example 308. Synthesis of Compound 764

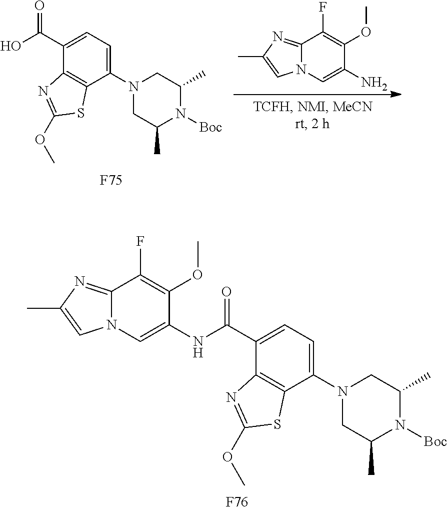

To a stirred mixture of 7-[(3S,5S)-4-tert-butoxycarbonyl-3,5-dimethyl-piperazin-1-yl]-2-methoxy-1,3-benzothiazole-4-carboxylic acid (Intermediate F75, 0.05 g, 118.62 μmol) and 8-fluoro-7-methoxy-2-methyl-imidazo[1,2-a]pyridin-6-amine (23.15 mg, 118.62 μmol) in MeCN (1 mL) were added NMI (9.4 mg, 118.62 μmol), TCFH (33.8 mg, 118.62 μmol) at room temperature. The resulting mixture was stirred for 2 h at room temperature. The resulting mixture was diluted with water (3 mL). The resulting mixture was extracted with EA (3×3 mL). The combined organic layers were washed with water (1×8 mL), brine (1×8 mL), dried over anhydrous Na₂SO₄. After filtration, the filtrate was concentrated under reduced pressure. The mixture was purified by silica gel column chromatography, eluted with PE/EA (1:8) to afford tert-butyl (2S,6S)-4-[4-[(8-fluoro-7-methoxy-2-methyl-imidazo[1,2-a]pyridin-6-yl)carbamoyl]-2-methoxy-1,3-benzothiazol-7-yl]-2,6-dimethyl-piperazine-1-carboxylate (Intermediate F76, 0.04 g, 66.81 μmol). LCMS (ES, m/z): 599 [M+H]⁺.

### Synthesis of Compound 764

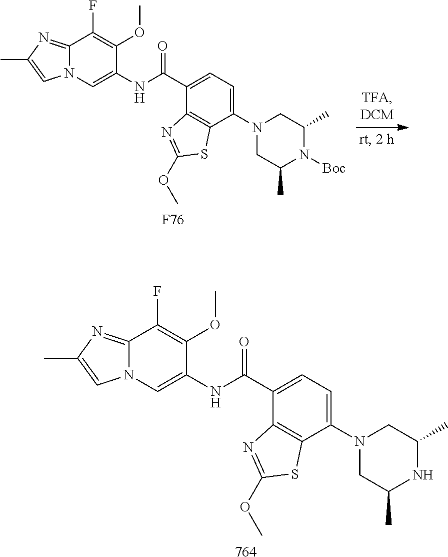

To a solution of tert-butyl (2S,6S)-4-[4-[(8-fluoro-7-methoxy-2-methyl-imidazo[1,2-a]pyridin-6-yl)carbamoyl]-2-methoxy-1,3-benzothiazol-7-yl]-2,6-dimethyl-piperazine-1-carboxylate (Intermediate F76, 0.04 g, 66.81 μmol) in DCM (1 mL) was added TFA (0.4 mL). The reaction was stirred for 1 h at rt. The resulting mixture was concentrated under reduced pressure and adjusted to pH 8 with saturated NaHCO₃ (aq.). After filtration, the filtrate was concentrated under reduced pressure. The residue was purified by reversed-phase flash chromatography with the following conditions (SunFire Prep C18 OBD Column 19*150 mm, 5 μm 10 nm, mobile phase, MeCN in water (0.05% TFA), 10% to 30% gradient in 7 min; detector, UV 254 nm) to afford 7-((3S,5S)-3,5-dimethylpiperazin-1-yl)-N-(8-fluoro-7-methoxy-2-methylimidazo[1,2-a]pyridin-6-yl)-2-methoxybenzo[d]thiazole-4-carboxamide 2,2,2-trifluoroacetate (Compound 764, 0.03 g, 48.97 μmol). LCMS (ES, m/z): 499 [M+H]⁺. ¹H NMR (400 MHz, DMSO-d₆) δ 11.63 (s, 1H), 9.69 (s, 1H), 9.20-9.01 (m, 2H), 8.24 (d, J=8.6 Hz, 1H), 8.04 (s, 1H), 7.23 (d, J=8.5 Hz, 1H), 4.42 (s, 3H), 4.26 (s, 3H), 3.79 (s, 2H), 3.45-3.35 (m, 2H), 3.26 (dd, J=13.1, 6.3 Hz, 2H), 2.42 (s, 3H), 1.42 (d, J=6.6 Hz, 6H).

### Example 309. Synthesis of Compound 767

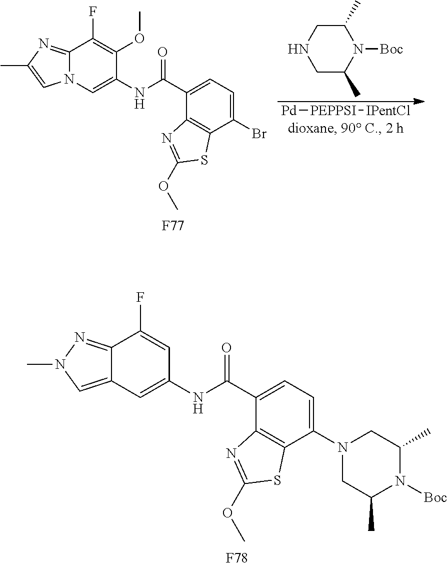

To a stirred mixture of 7-bromo-N-(7-fluoro-2-methyl-indazol-5-yl)-2-methoxy-1,3-benzothiazole-4-carboxamide (Intermediate F77, 60 mg, 137.85 μmol) and tert-butyl (2S,6S)-2,6-dimethylpiperazine-1-carboxylate (35.4 mg, 165.41 μmol) in DMF (1.5 mL) were added Cs₂CO₃ (89.8 mg, 275.69 μmol) and Pd-PEPPSI-IPentCl (11.5 mg, 13.78 μmol) at room temperature under nitrogen atmosphere. The resulting mixture was stirred for 2 h at 90° C. under nitrogen atmosphere. The resulting mixture was concentrated under reduced pressure. The residue was purified by silica gel column chromatography, eluted with PE/EA (1:3) to afford tert-butyl (2S,6S)-4-[4-[(7-fluoro-2-methyl-indazol-5-yl)carbamoyl]-2-methoxy-1,3-benzothiazol-7-yl]-2,6-dimethyl-piperazine-1-carboxylate (Intermediate F78, 40 mg, 70.34 μmol). LCMS (ES, m/z): 569 [M+H]⁺.

### Synthesis of Intermediate F79

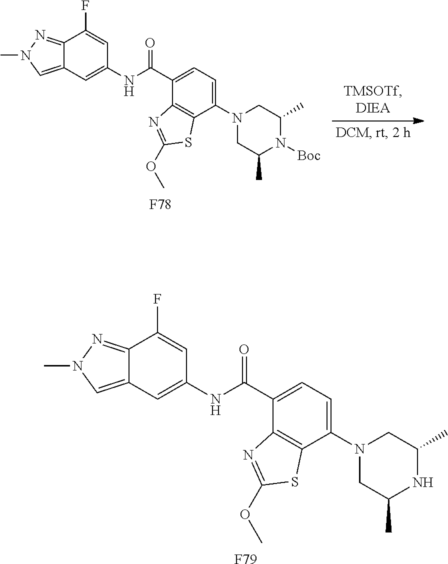

To a stirred mixture of tert-butyl (2S,6S)-4-[4-[(7-fluoro-2-methyl-indazol-5-yl)carbamoyl]-2-methoxy-1,3-benzothiazol-7-yl]-2,6-dimethyl-piperazine-1-carboxylate (Intermediate F78, 40 mg, 70.34 μmol) in DCM (1 mL) was added DIEA (18.1 mg, 140.68 μmol) and TMSOTf (46.9 mg, 211.02 μmol) dropwise at room temperature. The resulting mixture was stirred for 2 h at room temperature. The resulting mixture were concentrated under reduced pressure and diluted with water (2 mL). The mixture was basified to pH 8 with saturated NaHCO₃ (aq.) and extracted with DCM (3×3 mL). The combined organic layers were washed with water (2×10 mL) and brine (1×10 mL), dried over anhydrous Na₂SO₄. After filtration, the filtrate was concentrated under reduced pressure to afford 7-[(3S,5S)-3,5-dimethylpiperazin-1-yl]-N-(7-fluoro-2-methyl-indazol-5-yl)-2-methoxy-1,3-benzothiazole-4-carboxamide (Intermediate F79, 30 mg, 64.03 μmol, 91.03% yield) as a yellow solid. LCMS (ES, m/z): 469 [M+H]⁺.

### Synthesis of Compound 767

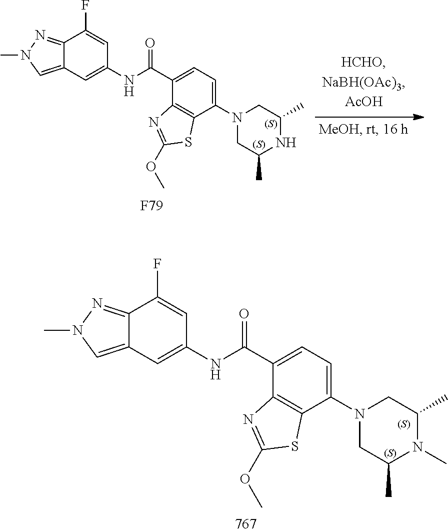

To a stirred mixture of 7-[(3S,5S)-3,5-dimethylpiperazin-1-yl]-N-(7-fluoro-2-methyl-indazol-5-yl)-2-methoxy-1,3-benzothiazole-4-carboxamide (Intermediate F79, 25 mg, 53.36 μmol) in MeOH (0.75 mL) was added HCHO (5.4 mg, 160.07 μmol) and AcOH (3.2 mg, 53.36 μmol) at room temperature. The resulting mixture was stirred for 16 h at room temperature. To the above mixture was added NaBH(OAc)₃ (33.9 mg, 160.07 μmol) at room temperature. The resulting mixture was stirred for 2 h at room temperature. The resulting mixture were concentrated under reduced pressure and diluted with water (5 mL). The mixture was basified to pH 8 with saturated NaHCO₃ (aq.) and extracted with DCM (3×5 mL). The combined organic layers were washed with water (2×10 mL) and brine (1×10 mL), dried over anhydrous Na₂SO₄. After filtration, the filtrate was concentrated under reduced pressure. The crude product was purified by Prep-HPLC with the following conditions (column, C18 silica gel, XBridge, 19×150 mm; mobile phase, MeCN in water (0.05% NH₃H₂O), 20% to 35% gradient in 7 min; detector, UV 254 nm) to afford N-(7-fluoro-2-methyl-indazol-5-yl)-2-methoxy-7-[(3S,5S)-3,4,5-trimethylpiperazin-1-yl]-1,3-benzothiazole-4-carboxamide (Compound 767, 8 mg, 16.58 μmol). LCMS (ES, m/z): 483 [M+H]⁺. ¹H NMR (400 MHz, DMSO-d₆) δ 11.41 (s, 1H), 8.44 (d, J=2.8 Hz, 1H), 8.16-8.08 (m, 2H), 7.36 (d, J=13.0 Hz, 1H), 7.07 (d, J=8.5 Hz, 1H), 4.42 (s, 3H), 4.19 (s, 3H), 3.16 (dd, J=12.0, 3.0 Hz, 2H), 3.03 (dd, J=11.5, 6.2 Hz, 2H), 2.91 (s, 2H), 2.27 (s, 3H), 1.09 (d, J=6.3 Hz, 6H).

### Example 310. Synthesis of Compound 771

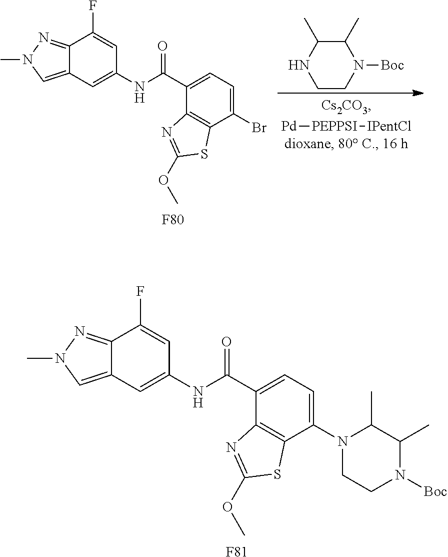

To a stirred mixture of 7-bromo-N-(7-fluoro-2-methyl-indazol-5-yl)-2-methoxy-1,3-benzothiazole-4-carboxamide (Intermediate F80, 140 mg, 321.64 μmol) in dioxane (1.5 mL) were added tert-butyl 2,3-dimethylpiperazine-1-carboxylate (82.7 mg, 385.97 μmol), Cs₂CO₃ (209.6 mg, 643.28 μmol) and Pd-PEPPSI-IPenCl (27.0 mg, 32.16 μmol) at rt under N₂. The mixture was stirred for 16 h at 80° C. under N₂. The reaction was diluted with H₂O (10 mL).

The resulting mixture was extracted with DCM (2×10 mL). The combined organic layers were washed with H₂O (10 mL), dried over anhydrous Na₂SO₄. After filtration, the filtrate was concentrated under reduced pressure. The residue was purified by silica gel column chromatography, eluted with DCM/EA (7/1) to afford tert-butyl 4-[4-[(7-fluoro-2-methyl-indazol-5-yl)carbamoyl]-2-methoxy-1,3-benzothiazol-7-yl]-2,3-dimethyl-piperazine-1-carboxylate (Intermediate F81, 105 mg, 184.64 μmol). LCMS (ES, m/z): 569 [M+H]⁺.

### Synthesis of Compound 771

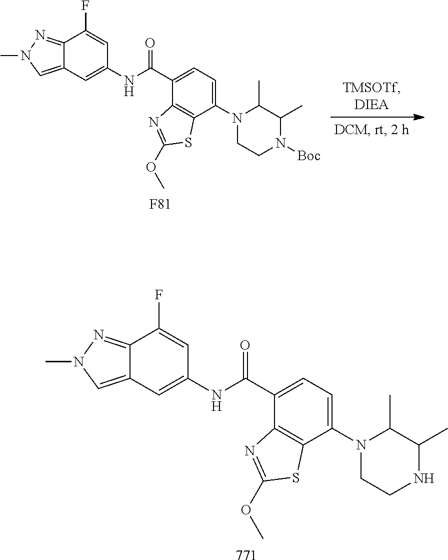

To a solution of tert-butyl 4-[4-[(7-fluoro-2-methyl-indazol-5-yl)carbamoyl]-2-methoxy-1,3-benzothiazol-7-yl]-2,3-dimethyl-piperazine-1-carboxylate (Intermediate F81, 105 mg, 184.64 μmol) in DCM (1.2 mL) were added DIEA (47.7 mg, 369.29 μmol) and TMSOTf (123.1 mg, 553.93 μmol). The mixture was stirred for 2 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 40% to 65% gradient in 7 min; detector, UV 254 nm) to afford 7-(2,3-dimethylpiperazin-1-yl)-N-(7-fluoro-2-methyl-indazol-5-yl)-2-methoxy-1,3-benzothiazole-4-carboxamide (Compound 771, 5 mg, 10.67 μmol). LCMS (ES, m/z): 469 [M+H]⁺. ¹H NMR (400 MHz, DMSO-d₆) δ 11.39 (s, 1H), 8.41 (d, J=2.8 Hz, 1H), 8.13 (d, J=22.3 Hz, 1H), 8.07 (d, J=1.6 Hz, 1H), 7.34 (d, J=13.1 Hz, 1H), 7.00 (d, J=8.6 Hz, 1H), 4.38 (s, 3H), 4.17 (s, 3H), 3.73 (dd, J=6.9, 3.3 Hz, 1H), 3.28-3.20 (m, 1H), 3.07 (dt, J=10.0, 5.0 Hz, 1H), 2.98 (d, J=11.8 Hz, 2H), 2.85-2.75 (m, 1H), 0.97 (d, J=6.6 Hz, 3H), 0.88 (d, J=6.5 Hz, 3H).

### Example 311. Synthesis of Compound 772

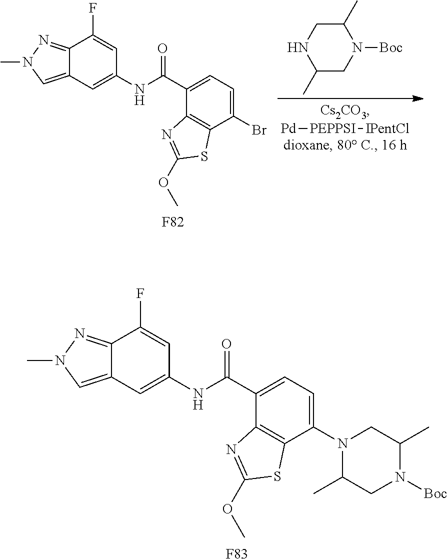

To a stirred mixture of 7-bromo-N-(7-fluoro-2-methyl-indazol-5-yl)-2-methoxy-1,3-benzothiazole-4-carboxamide (Intermediate F82, 90 mg, 206.77 μmol) in dioxane (1 mL) were added tert-butyl 2,5-dimethylpiperazine-1-carboxylate (53.1 mg, 248.12 μmol), Cs₂CO₃ (134.7 mg, 413.54 μmol) and Pd-PEPPSI-IPentCl (17.3 mg, 20.68 μmol) at rt under N₂. The mixture was stirred for 16 h at 80° C. under N₂. The reaction was diluted with H₂O (10 mL). The resulting mixture was extracted with DCM (2×10 mL). The combined organic layers were washed with H₂O (10 mL), dried over anhydrous Na₂SO₄. After filtration, the filtrate was concentrated under reduced pressure. The residue was purified by silica gel column chromatography, eluted with DCM/EA (1/1) to afford tert-butyl 4-[4-[(7-fluoro-2-methyl-indazol-5-yl)carbamoyl]-2-methoxy-1,3-benzothiazol-7-yl]-2,5-dimethyl-piperazine-1-carboxylate (Intermediate F83, 75 mg, 131.89 μmol). LCMS (ES, m/z): 569 [M+H]⁺.

### Synthesis of Compound 772

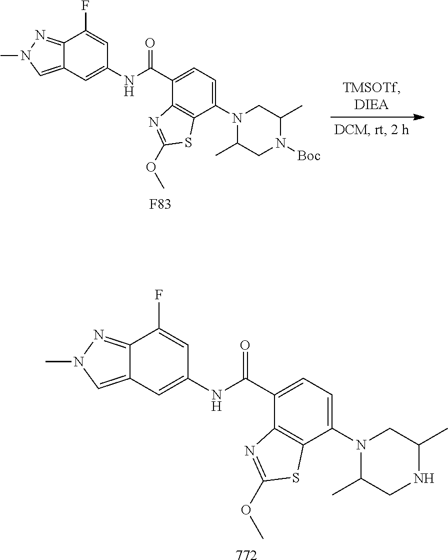

To a solution of tert-butyl 4-[4-[(7-fluoro-2-methyl-indazol-5-yl)carbamoyl]-2-methoxy-1,3-benzothiazol-7-yl]-2,5-dimethyl-piperazine-1-carboxylate (Intermediate F83, 75 mg, 131.89 μmol) in DCM (1 mL) were added DIEA (34.09 mg, 263.78 μmol) and TMSOTf (87.94 mg, 395.67 μmol). The mixture was stirred for 2 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 35% to 55% gradient in 7 min; detector, UV 254 nm) to afford 7-(2,5-dimethylpiperazin-1-yl)-N-(7-fluoro-2-methyl-indazol-5-yl)-2-methoxy-1,3-benzothiazole-4-carboxamide (Compound 772, 22 mg, 46.95 μmol). LCMS (ES, m/z): 469 [M+H]⁺. ¹H NMR (400 MHz, DMSO-d₆) δ 11.33 (s, 1H), 8.42 (d, J=2.9 Hz, 1H), 8.14 (d, J=8.4 Hz, 1H), 8.09 (d, J=1.6 Hz, 1H), 7.38-7.28 (m, 2H), 4.37 (s, 3H), 4.17 (s, 3H), 3.13 (ddd, J=9.6, 6.1, 3.1 Hz, 1H), 3.08-2.96 (m, 2H), 2.86 (ddd, J=9.6, 6.4, 3.1 Hz, 1H), 2.47 (s, 1H), 2.32 (t, J=10.5 Hz, 1H), 0.95 (d, J=6.3 Hz, 3H), 0.79 (d, J=6.0 Hz, 3H).

### Example 312. Synthesis of Compound 773

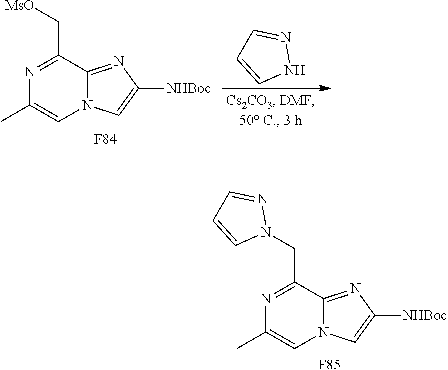

To a stirred solution of [2-(tert-butoxycarbonylamino)-6-methyl-imidazo[1,2-a]pyrazin-8-yl]methyl methanesulfonate (Intermediate F84, 150 mg, 420.88 μmol) and 1H-pyrazole (57.3 mg, 841.76 μmol) in DMF (3 mL) was added Cs₂CO₃ (411.3 mg, 1.26 mmol) at rt. The resulting mixture was stirred for 3 h at 50° C. The resulting mixture was diluted with water (10 mL). The resulting mixture was extracted with EA (3×10 mL). The combined organic layers were washed with water (2×10 mL) and brine (1×10 mL), dried over anhydrous Na₂SO₄. After filtration, the filtrate was concentrated under reduced pressure. The residue was purified by silica gel column chromatography, eluted with PE/EA (1:1) to afford tert-butyl N-[6-methyl-8-(pyrazol-1-ylmethyl)imidazo[1,2-a]pyrazin-2-yl]carbamate (Intermediate F85, 52 mg, 158.36 μmol). LCMS (ES, m/z): 329 [M+H]⁺.

### Synthesis of Intermediate F86

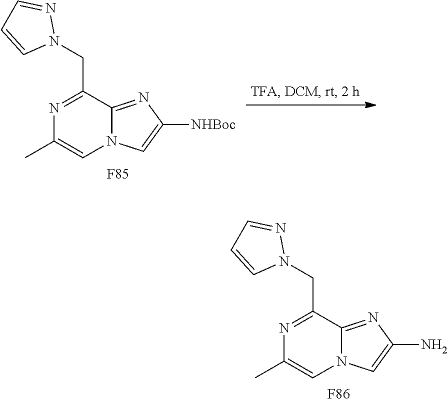

A solution of tert-butyl N-[6-methyl-8-(pyrazol-1-ylmethyl)imidazo[1,2-a]pyrazin-2-yl]carbamate (Intermediate F85, 47 mg, 143.13 μmol) in DCM (0.5 mL) was treated with TFA (0.5 mL) at room temperature. The resulting mixture was stirred for 2 h at room temperature. The resulting mixture was concentrated under reduced pressure. The resulting mixture was diluted with water (5 mL). The residue was basified to pH 8 with saturated NaHCO₃ (aq.). The resulting mixture was extracted with CH₂Cl₂:MeOH=10:1 (3×5 mL). The combined organic layers were washed with brine (1×5 mL), dried over anhydrous Na₂SO₄. After filtration, the filtrate was concentrated under reduced pressure to afford 6-methyl-8-(pyrazol-1-ylmethyl)imidazo[1,2-a]pyrazin-2-amine (Intermediate F86, 23 mg, 100.77 μmol). LCMS (ES, m/z): 229 [M+H]⁺.

### Synthesis of Intermediate F88

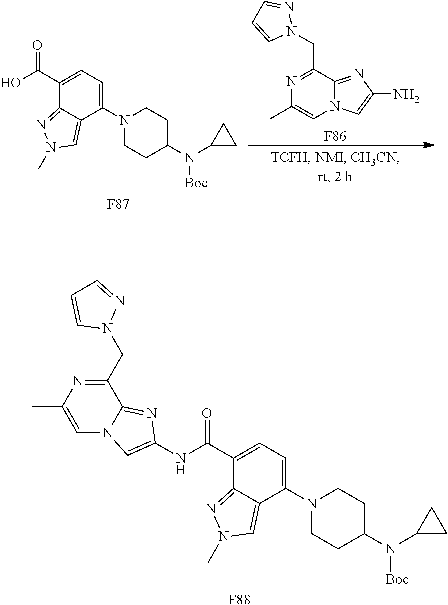

To a stirred mixture of 4-[4-[tert-butoxycarbonyl(cyclopropyl)amino]-1-piperidyl]-2-methyl-indazole-7-carboxylic acid (Intermediate F87, 30 mg, 72.38 μmol) and 6-methyl-8-(pyrazol-1-ylmethyl)imidazo[1,2-a]pyrazin-2-amine (Intermediate F86, 19.8 mg, 86.85 μmol) in CH₃CN (1 mL) were added NMI (17.0 mg, 217.13 μmol) and TCFH (30.4 mg, 108.57 μmol) at room temperature. The resulting mixture was stirred for 2 h at room temperature. The reaction was quenched by the addition of water (5 mL) at room temperature. The resulting mixture was extracted with EA (2×10 mL). The combined organic layers were washed with brine (1×8 mL), dried over anhydrous Na₂SO₄. After filtration, the filtrate was concentrated under reduced pressure. The residue was purified by silica gel column chromatography, eluted with EA to afford tert-butyl N-cyclopropyl-N-[1-[2-methyl-7-[[6-methyl-8-(pyrazol-1-ylmethyl)imidazo[1,2-a]pyrazin-2-yl]carbamoyl]indazol-4-yl]-4-piperidyl]carbamate (Intermediate F88, 27 mg, 43.22 μmol). LCMS (ES, m/z): 625 [M+H]⁺.

### Synthesis of Compound 773

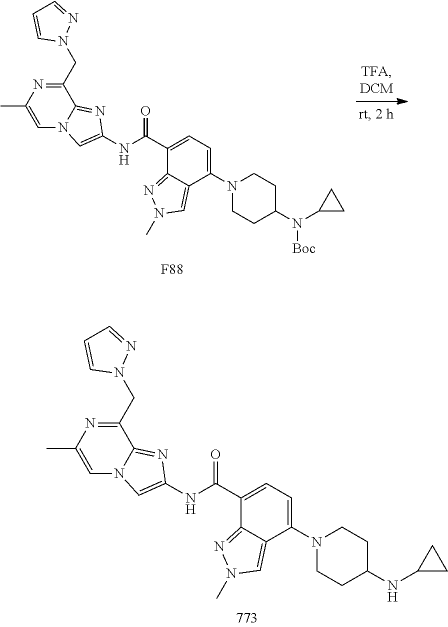

A solution of tert-butyl N-cyclopropyl-N-[1-[2-methyl-7-[[6-methyl-8-(pyrazol-1-ylmethyl)imidazo[1,2-a]pyrazin-2-yl]carbamoyl]indazol-4-yl]-4-piperidyl]carbamate (Intermediate F88, 27 mg, 43.22 μmol) in DCM (1 mL) was treated with TFA (1 mL) at room temperature. The resulting mixture was stirred for 2 h at room temperature under nitrogen atmosphere. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 50% to 70% gradient in 7 min; detector, UV 254 nm) to afford 4-[4-(cyclopropylamino)-1-piperidyl]-2-methyl-N-[6-methyl-8-(pyrazol-1-ylmethyl)imidazo[1,2-a]pyrazin-2-yl]indazole-7-carboxamide (Compound 773, 13 mg, 24.78 μmol). LCMS (ES, m/z): 525 [M+H]⁺. ¹H NMR (400 MHz, DMSO-d₆) δ 11.52 (s, 1H), 8.80 (s, 1H), 8.43 (d, J=8.2 Hz, 2H), 8.04 (d, J=8.1 Hz, 1H), 7.91 (d, J=2.3 Hz, 1H), 7.42 (d, J=1.8 Hz, 1H), 6.51 (d, J=8.2 Hz, 1H), 6.29 (t, J=2.0 Hz, 1H), 5.76 (s, 2H), 4.31 (s, 3H), 3.91 (d, J=12.8 Hz, 2H), 3.09 (t, J=11.7 Hz, 2H), 2.78 (d, J=10.6 Hz, 1H), 2.37 (s, 3H), 2.14 (td, J=6.6, 3.3 Hz, 1H), 2.07-1.96 (m, 2H), 1.49 (q, J=11.4 Hz, 2H), 0.40 (dt, J=6.2, 3.1 Hz, 2H), 0.24 (p, J=4.0 Hz, 2H).

### Example 313. Synthesis of Compound 774

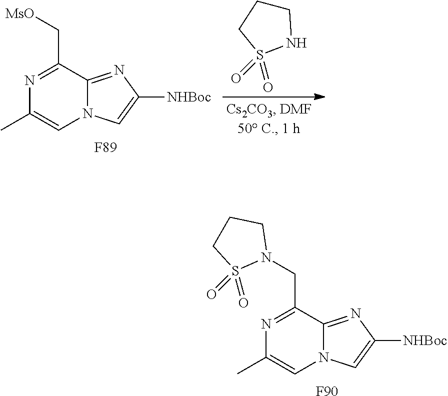

To a solution of [2-(tert-butoxycarbonylamino)-6-methyl-imidazo[1,2-a]pyrazin-8-yl]methyl methanesulfonate (Intermediate F89, 150 mg, 420.88 μmol) in DMF (2 mL) were added 1,2-thiazolidine 1,1-dioxide (76.5 mg, 631.32 μmol) and Cs₂CO₃ (411.4 mg, 1.26 mmol). The mixture was stirred for 1 h at 50° C. The reaction was diluted with H₂O (15 mL). The resulting mixture was extracted with EA (2×15 mL). The combined organic layers were washed with H₂O (2×25 mL), dried over anhydrous Na₂SO₄. After filtration, the filtrate was concentrated under reduced pressure. The residue was purified by silica gel column chromatography, eluted with PE/THF (2/3) to afford tert-butyl N-[8-[(1,1-dioxo-1,2-thiazolidin-2-yl)methyl]-6-methyl-imidazo[1,2-a]pyrazin-2-yl]carbamate (Intermediate F90, 60 mg, 157.29 μmol). LCMS (ES, m/z): 382 [M+H]⁺.

### Synthesis of Intermediate F91

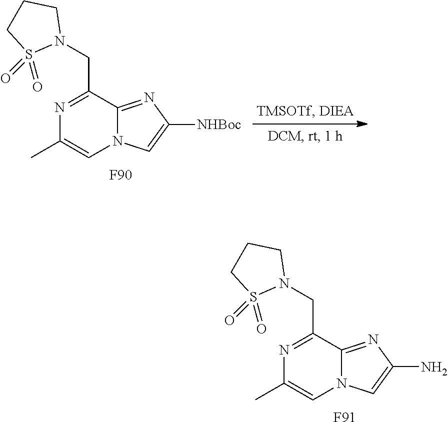

To a solution of tert-butyl N-[8-[(1,1-dioxo-1,2-thiazolidin-2-yl)methyl]-6-methyl-imidazo[1,2-a]pyrazin-2-yl]carbamate (Intermediate F90, 55 mg, 144.19 μmol) in DCM (0.6 mL) were added DIEA (37.2 mg, 288.37 μmol) and TMSOTf (96.1 mg, 432.56 μmol). The mixture was stirred for 1 h at rt. The reaction was diluted with NaHCO₃ (aq.) (5 mL). The resulting mixture was extracted with DCM/MeOH (3/1) (3×10 mL). The combined organic layers were dried over anhydrous Na₂SO₄. After filtration, the filtrate was concentrated under reduced pressure to afford 8-[(1,1-dioxo-1,2-thiazolidin-2-yl)methyl]-6-methyl-imidazo[1,2-a]pyrazin-2-amine (Intermediate F91, 50 mg, crude) as a brown solid. The crude product was used for next step without further purification. LCMS (ES, m/z): 282 [M+H]⁺.

### Synthesis of Intermediate F93

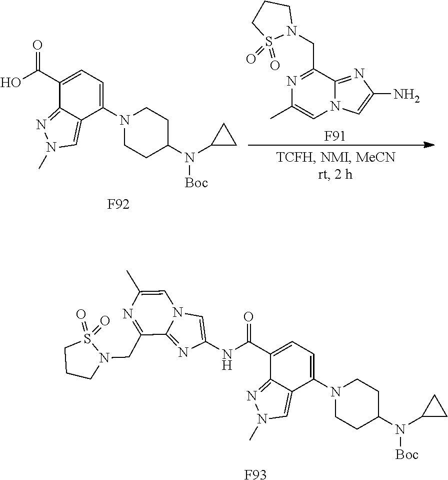

To a solution of 4-[4-[tert-butoxycarbonyl(cyclopropyl)amino]-1-piperidyl]-2-methyl-indazole-7-carboxylic acid (Intermediate F92, 40 mg, 96.50 μmol) in MeCN (0.5 mL) were added 8-[(1,1-dioxo-1,2-thiazolidin-2-yl)methyl]-6-methyl-imidazo[1,2-a]pyrazin-2-amine (Intermediate F91, 29.8 mg, 106.15 μmol), NMI (39.62 mg, 482.51 μmol) and TCFH (40.6 mg, 144.75 μmol). The mixture was stirred for 2 h at rt. The reaction was diluted with H₂O (10 mL). The resulting mixture was extracted with DCM (2×10 mL). The combined organic layers were washed with H₂O (10 mL), dried over anhydrous Na₂SO₄. After filtration, the filtrate was concentrated under reduced pressure. The residue was purified by silica gel column chromatography, eluted with DCM/EA (1/3) to afford tert-butyl N-cyclopropyl-N-[1-[7-[[8-[(1,1-dioxo-1,2-thiazolidin-2-yl)methyl]-6-methyl-imidazo[1,2-a]pyrazin-2-yl]carbamoyl]-2-methyl-indazol-4-yl]-4-piperidyl]carbamate (Intermediate F93, 35 mg, 51.64 μmol). LCMS (ES, m/z): 678 [M+H]⁺.

### Synthesis of Compound 774

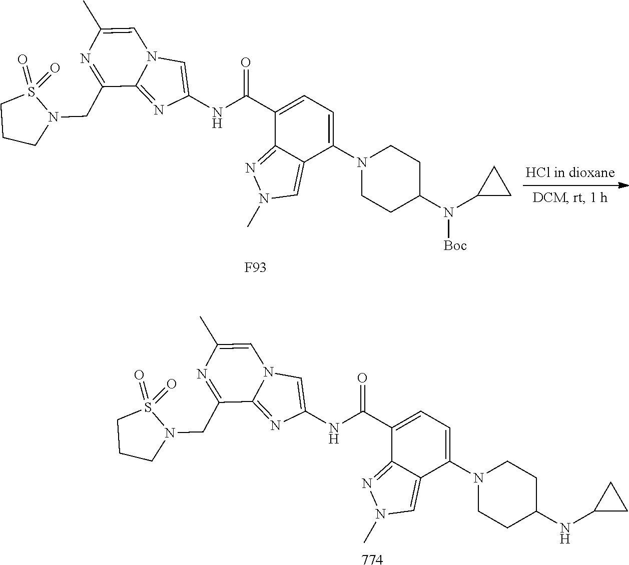

To a solution of tert-butyl N-cyclopropyl-N-[1-[7-[8-[(1,1-dioxo-1,2-thiazolidin-2-yl)methyl]-6-methyl-imidazo[1,2-a]pyrazin-2-yl]carbamoyl]-2-methyl-indazol-4-yl]-4-piperidyl]carbamate (Intermediate F93, 35 mg, 51.64 μmol) in DCM (0.5 mL) was added HCl (4.0 M in 1,4-dioxane) (129 μL). The mixture was stirred for 1 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 40% to 60% gradient in 7 min; detector, UV 254 nm) to afford 4-[4-(cyclopropylamino)-1-piperidyl]-N-[8-[(1,1-dioxo-1,2-thiazolidin-2-yl)methyl]-6-methyl-imidazo[1,2-a]pyrazin-2-yl]-2-methyl-indazole-7-carboxamide (Compound 774, 6 mg, 10.39 μmol). LCMS (ES, m/z): 578 [M+H]⁺. ¹H NMR (400 MHz, DMSO-d₆) δ 11.46 (s, 1H), 8.80 (s, 1H), 8.40 (d, J=12.3 Hz, 2H), 8.02 (d, J=8.1 Hz, 1H), 6.51 (d, J=8.2 Hz, 1H), 4.55 (s, 2H), 4.29 (s, 3H), 3.94 (d, J=12.6 Hz, 2H), 3.46 (t, J=6.8 Hz, 2H), 3.28-3.22 (m, 2H), 3.14-2.90 (m, 3H), 2.41 (s, 3H), 2.37-2.20 (m, 3H), 2.06 (d, J=12.5 Hz, 2H), 1.57 (d, J=13.3 Hz, 2H), 0.48 (d, J=36.0 Hz, 4H).

### Example 314. Synthesis of Compound 775

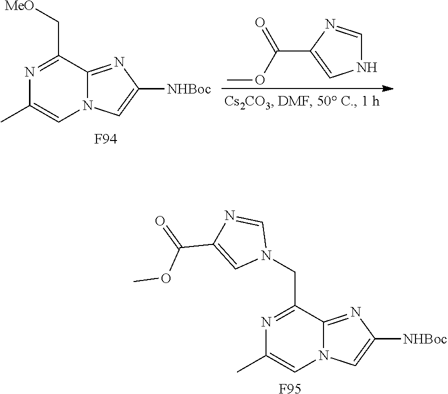

To a solution of [2-(tert-butoxycarbonylamino)-6-methyl-imidazo[1,2-a]pyrazin-8-yl]methyl methanesulfonate (Intermediate F94, 230 mg, 645.35 μmol) in DMF (2.5 mL) were added methyl 1H-imidazole-4-carboxylate (122.1 mg, 968.02 μmol) and Cs₂CO₃ (630.8 mg, 1.94 mmol). The mixture was stirred for 1 h at 50° C. The reaction was diluted with H₂O (15 mL). The resulting mixture was extracted with EA (2×15 mL). The combined organic layers were washed with H₂O (2×15 mL), dried over anhydrous Na₂SO₄. After filtration, the filtrate was concentrated under reduced pressure. The residue was purified by silica gel column chromatography, eluted with PE/THF (1/2) to afford methyl 1-[[2-(tert-butoxycarbonylamino)-6-methyl-imidazo[1,2-a]pyrazin-8-yl]methyl]imidazole-4-carboxylate (Intermediate F95, 73 mg, 188.92 μmol). LCMS (ES, m/z): 387 [M+H]⁺.

### Synthesis of Intermediate F96

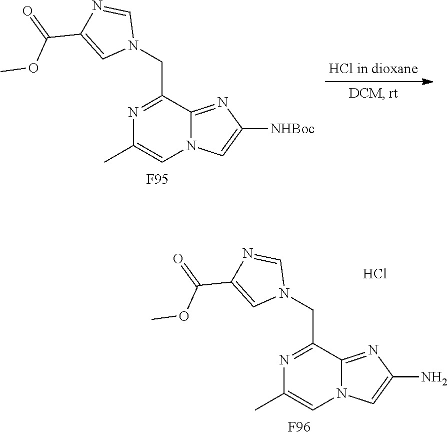

To a solution of methyl 1-[[2-(tert-butoxycarbonylamino)-6-methyl-imidazo[1,2-a]pyrazin-8-yl]methyl]imidazole-4-carboxylate (Intermediate F95, 70 mg, 181.16 μmol) in DCM (1 mL) was added HCl (4.0 M in 1,4-dioxane) (452 μL). The mixture was stirred for 2 h at rt. The resulting mixture was concentrated under reduced pressure to afford methyl 1-((2-amino-6-methylimidazo[1,2-a]pyrazin-8-yl)methyl)-1H-imidazole-4-carboxylate hydrochloride (Intermediate F96, 70 mg, crude). The crude product was used for next step without further purification. LCMS (ES, m/z): 287 [M+H]⁺.

### Synthesis of Intermediate F98

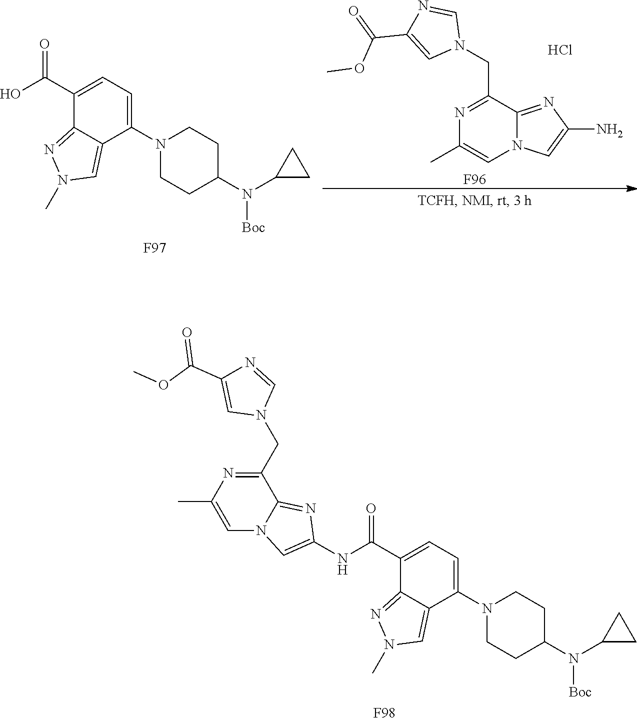

To a solution of 4-[4-[tert-butoxycarbonyl(cyclopropyl)amino]-1-piperidyl]-2-methyl-indazole-7-carboxylic acid (Intermediate F97, 60 mg, 144.75 μmol) in MeCN (1 mL) were added methyl 1-[(2-amino-6-methyl-imidazo[1,2-a]pyrazin-8-yl)methyl]imidazole-4-carboxylate (Intermediate F96, 45.6 mg, 159.23 μmol), NMI (59.4 mg, 723.77 μmol) and TCFH (60.9 mg, 217.13 μmol). The mixture was stirred for 3 h at rt. The reaction was diluted with H₂O (15 mL). The resulting mixture was extracted with DCM (2×15 mL). The combined organic layers were washed with H₂O (2×15 mL), dried over anhydrous Na₂SO₄. After filtration, the filtrate was concentrated under reduced pressure. The residue was purified by silica gel column chromatography, eluted with DCM/EA (1/2) to afford methyl 1-[[2-[[4-[4-[tert-butoxycarbonyl(cyclopropyl)amino]-1-piperidyl]-2-methyl-indazole-7-carbonyl]amino]-6-methyl-imidazo[1,2-a]pyrazin-8-yl]methyl]imidazole-4-carboxylate (Intermediate F98, 45 mg, 65.91 μmol). LCMS (ES, m/z): 683 [M+H]⁺.

### Synthesis of Intermediate F99

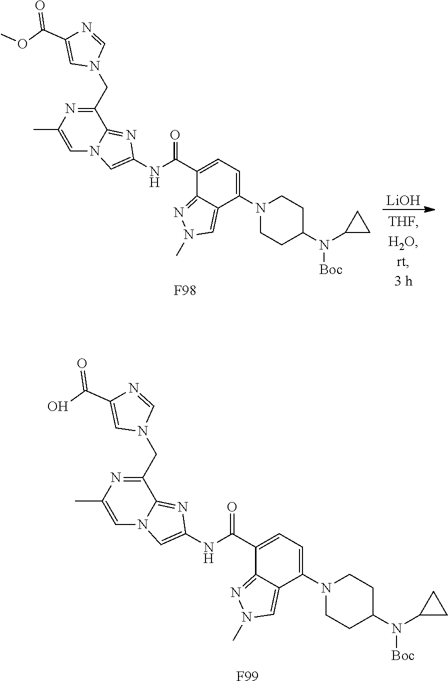

To a solution of methyl 1-[[2-[[4-[4-[tert-butoxycarbonyl(cyclopropyl)amino]-1-piperidyl]-2-methyl-indazole-7-carbonyl]amino]-6-methyl-imidazo[1,2-a]pyrazin-8-yl]methyl]imidazole-4-carboxylate (Intermediate F98, 45 mg, 65.91 μmol) in THF (0.4 mL)/H₂O (0.2 mL) was added LiOH·H₂O (13.83 mg, 329.54 μmol) at rt. The mixture was stirred for 3 h at rt. The resulting mixture was diluted with H₂O and adjusted to pH 5-6 with 1 N HCl. The resulting mixture was extracted with DCM (2×10 mL). The combined organic layers were washed with H₂O (10 mL), dried over anhydrous Na₂SO₄. The resulting mixture was concentrated under reduced pressure to afford 1-[[2-[[4-[4-[tert-butoxycarbonyl(cyclopropyl)amino]-1-piperidyl]-2-methyl-indazole-7-carbonyl]amino]-6-methyl-imidazo[1,2-a]pyrazin-8-yl]methyl]imidazole-4-carboxylic acid (Intermediate F99, 44 mg, 65.80 μmol). LCMS (ES, m/z): 669 [M+H]⁺.

### Synthesis of Intermediate F100

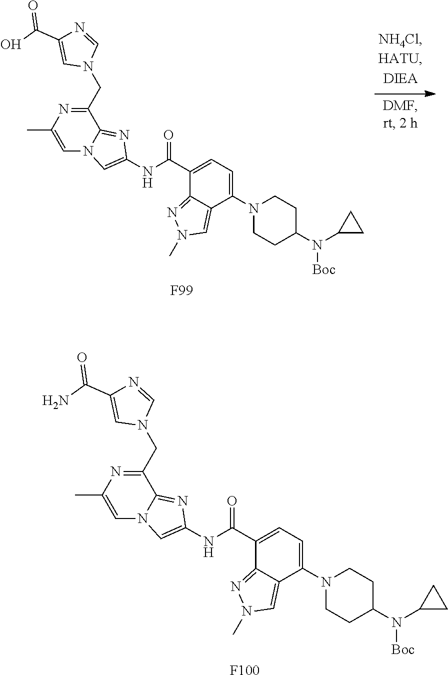

To a solution of 1-[[2-[[4-[4-[tert-butoxycarbonyl(cyclopropyl)amino]-1-piperidyl]-2-methyl-indazole-7-carbonyl]amino]-6-methyl-imidazo[1,2-a]pyrazin-8-yl]methyl]imidazole-4-carboxylic acid (Intermediate F99, 45 mg, 67.29 μmol) in DMF (0.6 mL) were added NH₄Cl (14.4 mg, 269.16 μmol), DIEA (43.5 mg, 336.45 μmol) and HATU (38.4 mg, 100.94 μmol). The mixture was stirred for 2 h at rt. The reaction was diluted with H₂O (10 mL). The resulting mixture was extracted with EA (2×10 mL). The combined organic layers were washed with H₂O (2×10 mL), dried over anhydrous Na₂SO₄. After filtration, the filtrate was concentrated under reduced pressure. The residue was purified by silica gel column chromatography, eluted with DCM/MeOH (12/1) to afford tert-butyl N-[1-[7-[[8-[(4-carbamoylimidazol-1-yl)methyl]-6-methyl-imidazo[1,2-a]pyrazin-2-yl]carbamoyl]-2-methyl-indazol-4-yl]-4-piperidyl]-N-cyclopropyl-carbamate (Intermediate F100, 20 mg, 29.95 μmol). LCMS (ES, m/z): 668 [M+H]⁻.

### Synthesis of Compound 775

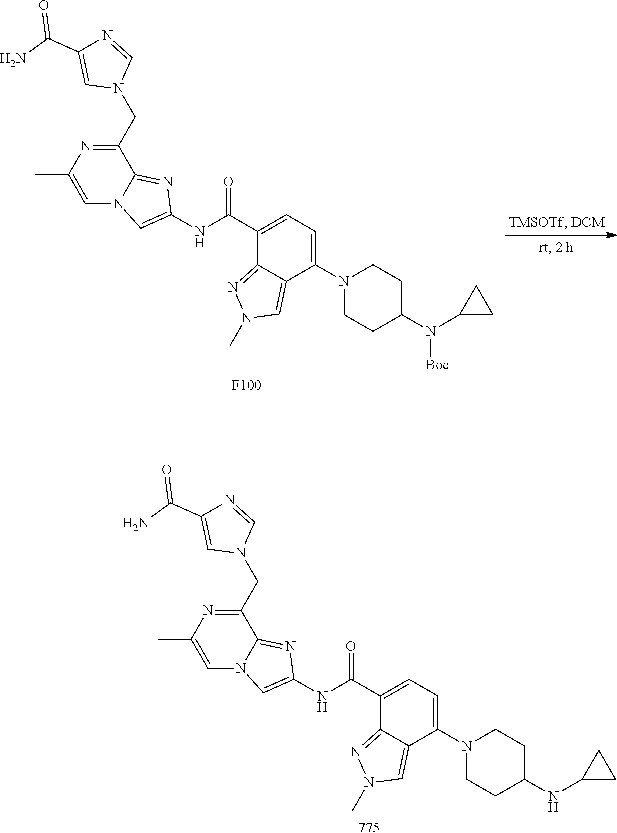

To a solution of tert-butyl N-[1-[7-[[8-[(4-carbamoylimidazol-1-yl)methyl]-6-methyl-imidazo[1,2-a]pyrazin-2-yl]carbamoyl]-2-methyl-indazol-4-yl]-4-piperidyl]-N-cyclopropyl-carbamate (Intermediate F100, 50 mg, 74.88 μmol) in DCM (1 mL) was added TFA (114 μL). The mixture was stirred for 1 h at rt. The resulting mixture was concentrated under reduced pressure. The crude product was purified by Prep-HPLC with the following conditions (column, C18 silica gel, XBridge, 19×150 mm; mobile phase, MeCN in water (0.05% TFA), 25% to 40% gradient in 7 min; detector, UV 254 nm) to afford N-(8-((4-carbamoyl-1H-imidazol-1-yl)methyl)-6-methylimidazo[1,2-a]pyrazin-2-yl)-4-(4-(cyclopropylamino)piperidin-1-yl)-2-methyl-2H-indazole-7-carboxamide 2,2,2-trifluoroacetate (Compound 775, 20 mg, 29.34 μmol). LCMS (ES, m/z): 568 [M+H]⁺. ¹H NMR (300 MHz, DMSO-d₆) δ 11.58 (s, 1H), 8.90 (s, 1H), 8.86 (s, 2H), 8.47 (d, J=9.9 Hz, 2H), 8.28 (s, 1H), 8.07 (d, J=8.1 Hz, 1H), 7.89 (s, 1H), 7.60 (s, 1H), 7.34 (s, 1H), 6.59 (d, J=8.2 Hz, 1H), 5.82 (s, 2H), 4.32 (s, 3H), 4.07 (d, J=12.9 Hz, 2H), 3.60-3.37 (m, 1H), 3.07 (t, J=12.4 Hz, 2H), 2.85-2.75 (m, 1H), 2.37 (d, J=1.0 Hz, 3H), 2.21 (d, J=11.7 Hz, 2H), 1.75 (d, J=8.2 Hz, 2H), 0.85 (d, J=5.6 Hz, 4H).

### Example 315. Synthesis of Compound 777

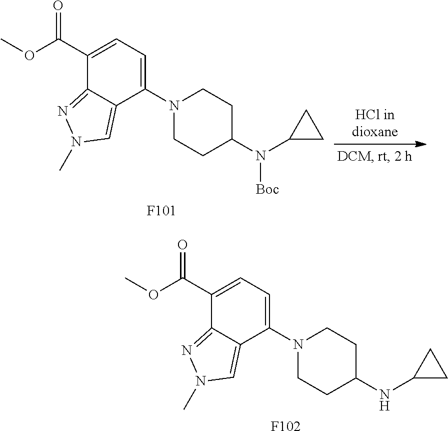 [SMILES: COC(=O)c1ccc(N2CCC(N(C(=O)OC(C)(C)C)C3CC3)CC2)c2cn(C)nc12 ; COC(=O)c1ccc(N2CCC(NC3CC3)CC2)c2cn(C)nc12 ; "F101" ; "F102"]

A solution of methyl 4-[4-[tert-butoxycarbonyl(cyclopropyl)amino]-1-piperidyl]-2-methyl-indazole-7-carboxylate (Intermediate F101, 150 mg, 350.04 μmol) in DCM (1 mL) was treated with HCl (4.0 M in 1,4-dioxane) (1 mL) at room temperature. The resulting mixture was stirred for 2 h at room temperature under nitrogen atmosphere. The resulting mixture was concentrated under reduced pressure. The resulting mixture was diluted with water (10 mL). The residue was basified to pH 8 with saturated NaHCO₃ (aq.). The resulting mixture was extracted with CH₂Cl₂:MeOH=10:1 (2×20 mL). The combined organic layers were washed with brine (1×20 mL), dried over anhydrous Na₂SO₄. After filtration, the filtrate was concentrated under reduced pressure to afford methyl 4-[4-(cyclopropylamino)-1-piperidyl]-2-methyl-indazole-7-carboxylate (Intermediate F102, 106 mg, 322.77 μmol). LCMS (ES, m/z): 329 [M+H]⁺.

### Synthesis of Intermediate F103

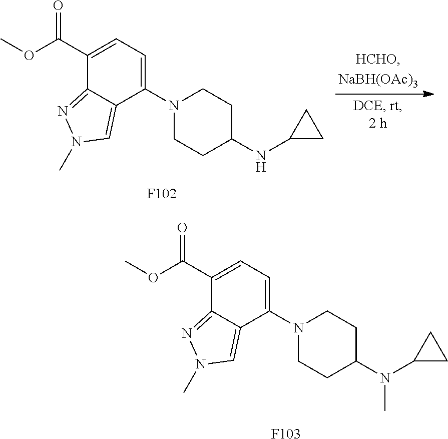 [SMILES: COC(=O)c1ccc(N2CCC(NC3CC3)CC2)c2cn(C)nc12 ; COC(=O)c1ccc(N2CCC(N(C)C3CC3)CC2)c2cn(C)nc12 ; "F102" ; "F103"]

A solution of methyl 4-[4-(cyclopropylamino)-1-piperidyl]-2-methyl-indazole-7-carboxylate (Intermediate F102, 106 mg, 322.77 μmol) in DCE (2 mL) as treated with HCHO (21.9 mg, 645.54 μmol) followed by the addition of NaBH(OAc)₃ (136.8 mg, 645.54 μmol) at room temperature. The resulting mixture was stirred for 2 h at room temperature. The resulting mixture was diluted with water (10 mL). The resulting mixture was extracted with DCM:MeOH=10:1 (3×10 mL). The combined organic layers were washed with brine (1×20 mL), dried over anhydrous Na₂SO₄. After filtration, the filtrate was concentrated under reduced pressure. The residue was purified by silica gel column chromatography, eluted with DCM/MeOH (5:1) to afford methyl 4-[4-[cyclopropyl(methyl)amino]-1-piperidyl]-2-methyl-indazole-7-carboxylate (Intermediate F103, 86 mg, 251.14 μmol). LCMS (ES, m/z): 343 [M+H]⁺.

### Synthesis of Intermediate F104

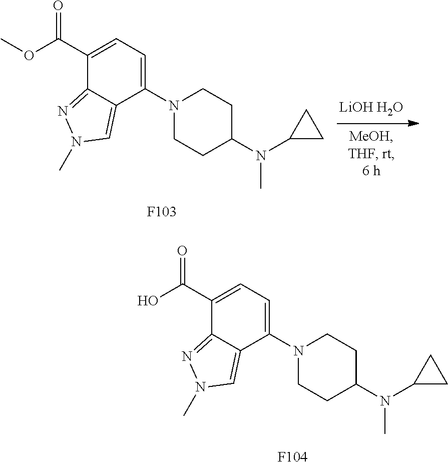

A solution of methyl 4-[4-[cyclopropyl(methyl)amino]-1-piperidyl]-2-methyl-indazole-7-carboxylate (Intermediate F103, 86 mg, 251.14 μmol) in THF (0.5 mL), MeOH (0.5 mL) and H₂O (0.5 mL) was treated with LiOH·H₂O (52.6 mg, 1.26 mmol) at room temperature. The resulting mixture was stirred for 6 h at rt. The mixture was acidified to pH 3 with HCl (1 N). The resulting mixture was extracted with EA (3×20 mL). The combined organic layers were washed with brine (1×40 mL), dried over anhydrous Na₂SO₄. After filtration, the filtrate was concentrated under reduced pressure. The residue was purified by silica gel column chromatography, eluted with DCM/MeOH (5:1) to afford 4-[4-[cyclopropyl(methyl)amino]-1-piperidyl]-2-methyl-indazole-7-carboxylic acid (Intermediate F104, 52 mg, 158.34 μmol). LCMS (ES, m/z): 329 [M+H]⁺.

### Synthesis of Compound 777

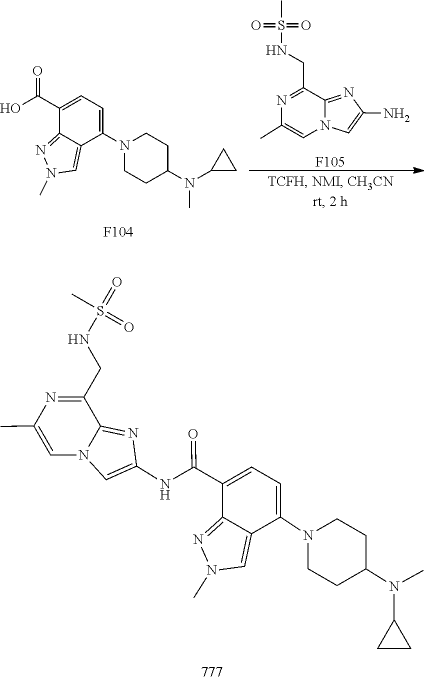

To a stirred mixture of N-[(2-amino-6-methyl-imidazo[1,2-a]pyrazin-8-yl)methyl]methanesulfonamide (Intermediate F105, 39.1 mg, 153.47 μmol) and 4-[4-[cyclopropyl(methyl)amino]-1-piperidyl]-2-methyl-indazole-7-carboxylic acid (Intermediate F104, 42 mg, 127.89 μmol) in CH₃CN (1 mL) were added NMI (31.5 mg, 383.67 μmol) and TCFH (53.8 mg, 191.83 μmol) at room temperature. The resulting mixture was stirred for 2 h at rt. The resulting mixture was diluted with water (10 mL). The resulting mixture was extracted with DCM:MeOH=10:1 (2×20 mL). The combined organic layers were washed with brine (1×10 mL), dried over anhydrous Na₂SO₄. After filtration, the filtrate was concentrated under reduced pressure. The crude product was purified by Prep-HPLC with the following conditions (column, C18 silica gel, XBridge, 19×150 mm; mobile phase, MeCN in water (0.05% NH₃·H₂O), 35% to 75% gradient in 7 min; detector, UV 254 nm) to afford 4-[4-cyclopropyl(methyl)amino]-1-piperidyl]-N-[8-(methanesulfonamidomethyl)-6-methyl-imidazo[1,2-a]pyrazin-2-yl]-2-methyl-indazole-7-carboxamide (Compound 777, 16 mg, 28.28 μmol). LCMS (ES, m/z): 566 [M+H]⁺. ¹H NMR (400 MHz, DMSO-d₆) δ 11.51 (s, 1H), 8.81 (s, 1H), 8.44 (s, 1H), 8.41 (s, 1H), 8.04 (d, J=8.1 Hz, 1H), 7.49 (t, J=5.9 Hz, 1H), 6.52 (d, J=8.2 Hz, 1H), 4.67 (d, J=5.5 Hz, 2H), 4.30 (s, 3H), 4.02 (d, J=12.6 Hz, 2H), 3.07 (s, 3H), 2.99 (t, J=12.2 Hz, 2H), 2.75-2.63 (m, 1H), 2.44 (s, 3H), 2.31 (s, 3H), 1.96 (d, J=12.3 Hz, 2H), 1.84 (dt, J=6.7, 3.1 Hz, 1H), 1.77-1.62 (m, 2H), 0.49 (h, J=4.2 Hz, 2H), 0.33 (p, J=4.0 Hz, 2H).

### Example 316. Synthesis of Compound 782

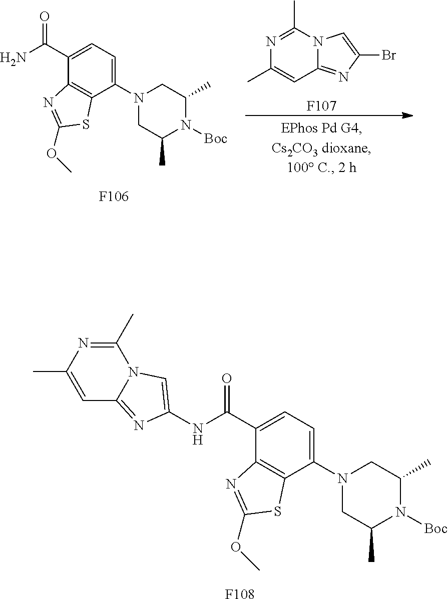

To a stirred mixture of 2-bromo-5,7-dimethyl-imidazo[1,2-c]pyrimidine (Intermediate F107, 32.2 mg, 142.68 μmol) and tert-butyl (2S,6S)-4-(4-carbamoyl-2-methoxy-1,3-benzothiazol-7-yl)-2,6-dimethyl-piperazine-1-carboxylate (Intermediate F106, 50 mg, 118.90 μmol) in dioxane (2 mL) were added Cs₂CO₃ (18.5 mg, 237.80 μmol) and EPhos Pd G4 (10.9 mg, 11.89 μmol) at room temperature under nitrogen atmosphere. The resulting mixture was stirred for 2 h at 100° C. under nitrogen atmosphere. The mixture was allowed to cool down to room temperature. The resulting mixture was concentrated under reduced pressure. The residue was purified by silica gel column chromatography, eluted with PE/EA (1:1) to afford tert-butyl (2S,6S)-4-[4-[(5,7-dimethylimidazo[1,2-c]pyrimidin-2-yl)carbamoyl]-2-methoxy-1,3-benzothiazol-7-yl]-2,6-dimethyl-piperazine-1-carboxylate (Intermediate F108, 37 mg, 65.41 μmol). LCMS (ES, m/z): 566 [M+H]⁺.

### Synthesis of Compound 782

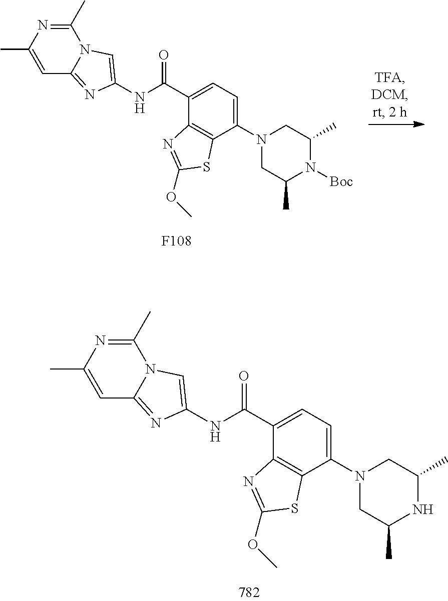

To a stirred mixture of tert-butyl (2S,6S)-4-[4-[(5,7-dimethylimidazo[1,2-c]pyrimidin-2-yl)carbamoyl]-2-methoxy-1,3-benzothiazol-7-yl]-2,6-dimethyl-piperazine-1-carboxylate (Intermediate F108, 37 mg, 65.41 μmol) in DCM (0.5 mL) was added TFA (0.5 mL) dropwise at room temperature. The resulting mixture was stirred for 2 h at room temperature. The resulting mixture was concentrated under reduced pressure. The crude product was purified by Prep-HPLC with the following conditions (column, C18 silica gel, XBridge, 19×150 mm; mobile phase, MeCN in water (0.05% NH₃·H₂O), 40% to 85% gradient in 14 min; detector, UV 254 nm) to afford N-(5,7-dimethylimidazo[1,2-c]pyrimidin-2-yl)-7-[(3S,5S)-3,5-dimethylpiperazin-1-yl]-2-methoxy-1,3-benzothiazole-4-carboxamide (Compound 782, 15 mg, 32.22 μmol). LCMS (ES, m/z): 466 [M+H]⁺. ¹H NMR (400 MHz, DMSO-d₆) δ 11.90 (s, 1H), 8.15 (d, J=8.5 Hz, 1H), 8.00 (s, 1H), 7.21 (s, 1H), 7.05 (d, J=8.6 Hz, 1H), 4.36 (s, 3H), 3.23 (td, J=6.2, 3.1 Hz, 2H), 3.12 (dd, J=11.5, 3.1 Hz, 2H), 2.92 (dd, J=11.6, 6.1 Hz, 2H), 2.75 (s, 3H), 2.39 (s, 3H), 1.16 (d, J=6.4 Hz, 6H).

### Example 317. Synthesis of Compound 783

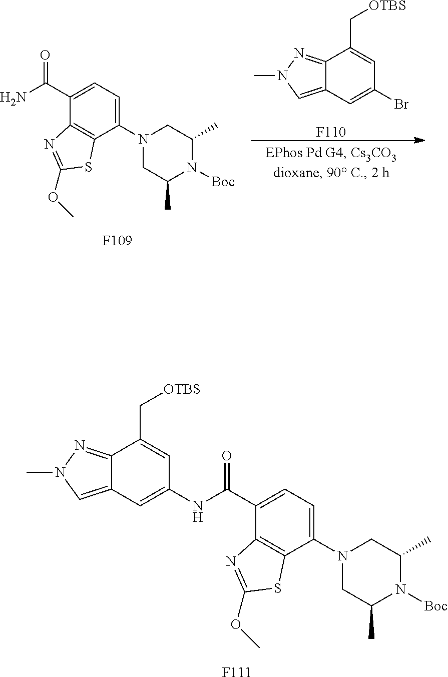

To a stirred mixture of tert-butyl (2S,6S)-4-(4-carbamoyl-2-methoxybenzo[d]thiazol-7-yl)-2,6-dimethylpiperazine-1-carboxylate (Intermediate F109, 50 mg, 118.90 μmol) and 5-bromo-7-(((tert-butyldimethylsilyl)oxy)methyl)-2-methyl-2H-indazole (Intermediate F110, 50.7 mg, 142.68 μmol) in dioxane (1 mL) were added Cs₂CO₃ (77.5 mg, 237.80 μmol) and EPhos Pd G4 (10.9 mg, 11.89 μmol) at room temperature under nitrogen atmosphere. The resulting mixture was stirred for 2 h at 90° C. under nitrogen atmosphere. The mixture was concentrated under reduced pressure. The residue was purified by silica gel column chromatography, eluted with DCM/MeOH (20:1) to afford tert-butyl (2S,6S)-4-(4-((7-(((tert-butyldimethylsilyl)oxy)methyl)-2-methyl-2H-indazol-5-yl)carbamoyl)-2-methoxybenzo[d]thiazol-7-yl)-2,6-dimethylpiperazine-1-carboxylate (Intermediate F111, 50 mg, 71.95 μmol, 60.51% yield) as an off-white solid. LCMS (ES, m/z): 695 [M+H]⁺.

### Synthesis of Compound 783

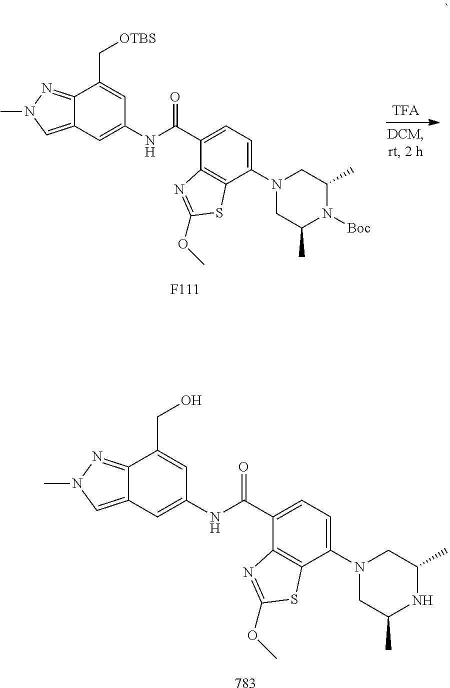

To a stirred mixture of tert-butyl (2S,6S)-4-(4-((7-(((tert-butyldimethylsilyl)oxy)methyl)-2-methyl-2H-indazol-5-yl)carbamoyl)-2-methoxybenzo[d]thiazol-7-yl)-2,6-dimethylpiperazine-1-carboxylate (Intermediate F111, 50 mg, 71.95 μmol) in DCM (1 mL) was added TFA (0.5 mL) at room temperature. The resulting mixture was stirred for 2 h at rt. The mixture was concentrated under reduced pressure. The residue was purified by Prep-HPLC with the following conditions (Column: XBridge Prep OBD Column 19×150 mm 8 μm; Mobile Phase A: water (0.05% NH₃·H₂O), Mobile Phase B: ACN; Flow rate: 20 mL/min; Gradient: 50% B to 95% B in 8 min, 90% B; Wave Length: 220 nm; RT1 (min): 7.2) to afford 7-((3S,5S)-3,5-dimethylpiperazin-1-yl)-N-(7-(hydroxymethyl)-2-methyl-2H-indazol-5-yl)-2-methoxybenzo[d]thiazole-4-carboxamide (Compound 783, 21.3 mg, 44.32 μmol). LCMS (ES, m/z): 481 [M+H]⁺. ¹H NMR (400 MHz, DMSO-d₆) δ 11.56 (s, 1H), 8.29 (d, J=4.4 Hz, 2H), 8.15 (d, J=8.5 Hz, 1H), 7.34 (s, 1H), 7.07 (d, J=8.6 Hz, 1H), 5.26 (t, J=5.9 Hz, 1H), 4.88 (d, J=5.3 Hz, 2H), 4.42 (s, 3H), 4.15 (s, 3H), 3.24 (s, 2H), 3.12 (d, J=10.8 Hz, 2H), 2.92 (dd, J=11.5, 6.0 Hz, 2H), 1.18 (d, J=6.4 Hz, 6H).

### Example 318. Synthesis of Compound 784

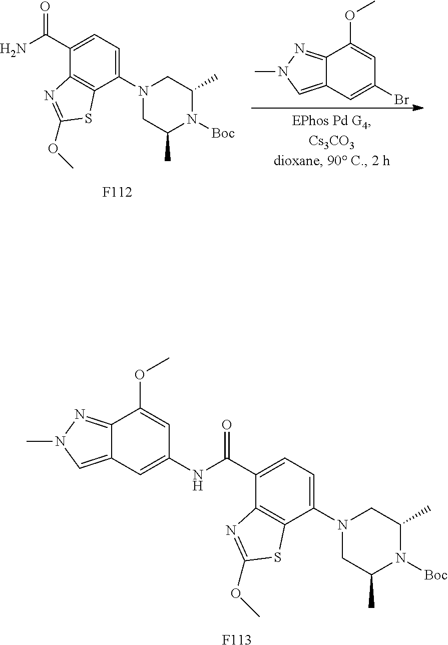

To a stirred mixture of tert-butyl (2S,6S)-4-(4-carbamoyl-2-methoxy-1,3-benzothiazol-7-yl)-2,6-dimethyl-piperazine-1-carboxylate (Intermediate F112, 50 mg, 118.90 μmol) and 5-bromo-7-methoxy-2-methyl-2H-indazole (34.4 mg, 142.68 μmol) in dioxane (0.5 mL) were added Cs₂CO₃ (77.5 mg, 237.80 μmol) and EPhos Pd G4 (10.9 mg, 11.89 μmol) at room temperature under nitrogen atmosphere. The resulting mixture was stirred for 2 h at 90° C. under nitrogen atmosphere. The resulting mixture was removed under reduced pressure. The residue was purified by silica gel column chromatography, eluted with DCM/MeOH (20:1) to afford tert-butyl (2S,6S)-4-[2-methoxy-4-[(7-methoxy-2-methyl-indazol-5-yl)carbamoyl]-1,3-benzothiazol-7-yl]-2,6-dimethyl-piperazine-1-carboxylate (Intermediate F113, 50 mg, 86.10 μmol). LCMS (ES, m/z): 581 [M+H]⁻.

### Synthesis of Compound 784

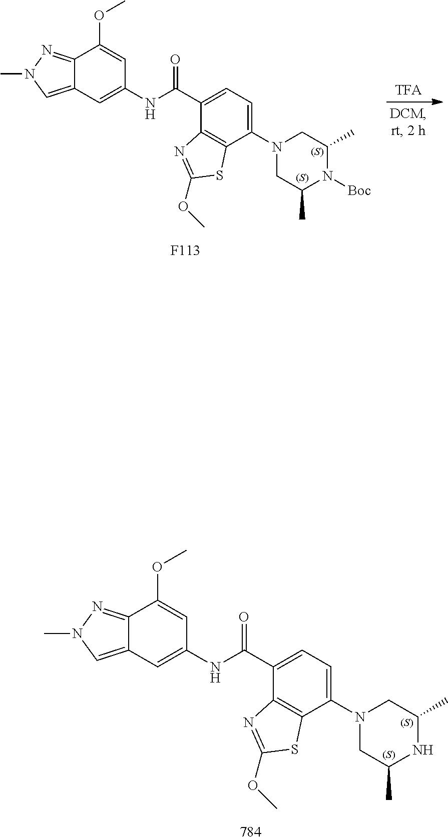

To a stirred mixture of tert-butyl (2S,6S)-4-(2-methoxy-4-((7-methoxy-2-methyl-2H-indazol-5-yl)carbamoyl)benzo[d]thiazol-7-yl)-2,6-dimethylpiperazine-1-carboxylate (Intermediate F113, 50 mg, 86.10 μmol) in DCM (0.5 mL) was added TFA (0.1 mL) at room temperature. The resulting mixture was stirred for 2 h at rt. The mixture was concentrated under reduced pressure. The residue was purified by Prep-HPLC with the following conditions (Column: XBridge Prep OBD Column 19×150 mm 8 μm; Mobile Phase A: water (0.05% NH₃·H₂O), Mobile Phase B: ACN; Flow rate: 20 mL/min; Gradient: 50% B to 95% B in 8 min, 90% B; Wave Length: 220 nm; RT1 (min): 7.2) to afford 7-((3S,5S)-3,5-dimethylpiperazin-1-yl)-2-methoxy-N-(7-methoxy-2-methyl-2H-indazol-5-yl)benzo[d]thiazole-4-carboxamide (Compound 784, 35.6 mg, 74.08 μmol). LCMS (ES, m/z): 481 [M+H]⁺. ¹H NMR (400 MHz, DMSO-d₆) δ 11.48 (s, 1H), 8.24 (s, 1H), 8.13 (d, J=8.4 Hz, 1H), 7.91 (s, 1H), 7.05 (d, J=8.6 Hz, 1H), 6.76 (d, J=1.9 Hz, 1H), 4.44 (s, 3H), 4.12 (s, 3H), 3.92 (s, 3H), 3.28-3.19 (m, 2H), 3.10 (dd, J=11.5, 3.1 Hz, 2H), 2.92 (dd, J=11.5, 6.1 Hz, 2H), 1.17 (d, J=6.4 Hz, 6H).

### Example 319. Synthesis of Compound 787

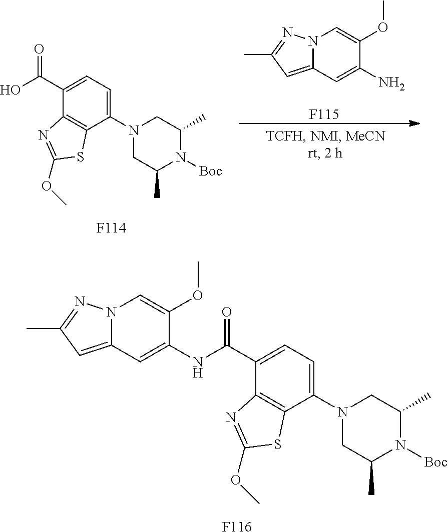

To a stirred mixture of 7-[(3S,5S)-4-tert-butoxycarbonyl-3,5-dimethyl-piperazin-1-yl]-2-methoxy-1,3-benzothiazole-4-carboxylic acid (Intermediate F114, 0.05 g, 118.62 μmol) and 6-methoxy-2-methyl-pyrazolo[1,5-a]pyridin-5-amine (Intermediate F115, 25.2 mg, 142.35 μmol) in MeCN (1 mL) were added NMI (29.2 mg, 355.86 μmol), TCFH (49.9 mg, 177.93 μmol) at room temperature. The resulting mixture was stirred for 2 h at room temperature. The resulting mixture was diluted with water (3 mL). The resulting mixture was extracted with EA (3×3 mL). The combined organic layers were washed with water (1×8 mL), brine (1×8 mL), dried over anhydrous Na₂SO₄. After filtration, the filtrate was concentrated under reduced pressure. The mixture was purified by silica gel column chromatography, eluted with PE/EA (1:8) to afford tert-butyl (2S,6S)-4-[2-methoxy-4-[(6-methoxy-2-methyl-pyrazolo[1,5-a]pyridin-5-yl)carbamoyl]-1,3-benzothiazol-7-yl]-2,6-dimethyl-piperazine-1-carboxylate (Intermediate F116, 0.04 g, 68.88 μmol). LCMS (ES, m/z): 581 [M+H]⁺.

### Synthesis of Compound 787

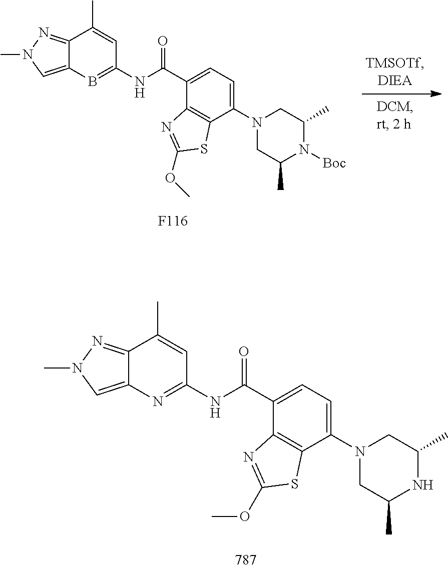

To a solution of tert-butyl (2S,6S)-4-[2-methoxy-4-[(6-methoxy-2-methyl-pyrazolo[1,5-a]pyridin-5-yl)carbamoyl]-1,3-benzothiazol-7-yl]-2,6-dimethyl-piperazine-1-carboxylate (Intermediate F116, 0.04 g, 68.88 μmol) in DCM (1 mL) was added TFA (0.4 mL). The reaction was stirred for 1 h at rt. The resulting mixture was concentrated under reduced pressure and adjusted to pH 8 with saturated NaHCO₃ (aq.). After filtration, the filtrate was concentrated under reduced pressure. The residue was purified by reversed-phase flash chromatography with the following conditions (SunFire Prep C18 OBD Column 19*150 mm, 5 μm 10 nm, mobile phase, MeCN in water (0.05% TFA), 10% to 30% gradient in 7 min; detector, UV 254 nm) to afford 7-((3S,5S)-3,5-dimethylpiperazin-1-yl)-2-methoxy-N-(6-methoxy-2-methylpyrazolo[1,5-a]pyridin-5-yl)benzo[d]thiazole-4-carboxamide 2,2,2-trifluoroacetate (Compound 787, 0.02 g, 33.64 μmol). LCMS (ES, m/z): 481 [M+H]⁺. ¹H NMR (400 MHz, DMSO-d₆) δ 11.51 (d, J=3.0 Hz, 1H), 8.97 (s, 2H), 8.67 (s, 1H), 8.40 (d, J=6.2 Hz, 1H), 8.23 (dd, J=8.7, 2.8 Hz, 1H), 7.19 (dd, J=9.4, 3.4 Hz, 1H), 6.27 (s, 1H), 4.42 (d, J=2.9 Hz, 3H), 3.90 (d, J=4.6 Hz, 2H), 3.82-3.70 (m, 2H), 3.39-3.31 (m, 2H), 3.23 (dd, J=13.0, 6.3 Hz, 2H), 2.34 (s, 3H), 1.41 (d, J=6.5 Hz, 6H).

### Example 320. Synthesis of Compound 791

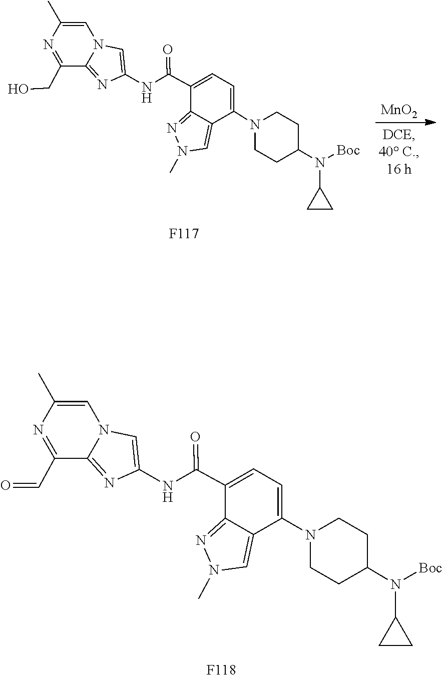 [SMILES: Cc1cn2cc(NC(=O)c3ccc(N4CCC(N(C(=O)OC(C)(C)C)C5CC5)CC4)c4cn(C)nc34)nc2c(CO)n1 ; "MnO2" ; Cc1cn2cc(NC(=O)c3ccc(N4CCC(N(C(=O)OC(C)(C)C)C5CC5)CC4)c4cn(C)nc34)nc2c(C=O)n1 ; "F117" ; "F118"]

To a stirred mixture of tert-butyl N-cyclopropyl-N-[1-[7-[[8-(hydroxymethyl)-6-methyl-imidazo[1,2-a]pyrazin-2-yl]carbamoyl]-2-methyl-indazol-4-yl]-4-piperidyl]carbamate (Intermediate F117, 80 mg, 139.21 μmol) in DCE (1.60 mL) was added MnO₂ (121.0 mg, 1.39 mmol) at room temperature. The resulting mixture was stirred for 16 h at 40° C. Filtered through a Celite pad and concentrated under reduced pressure to afford tert-butyl N-cyclopropyl-N-[1-[7-[(8-formyl-6-methyl-imidazo[1,2-a]pyrazin-2-yl)carbamoyl]-2-methyl-indazol-4-yl]-4-piperidyl]carbamate (Intermediate F118, 65 mg, 113.51 μmol). LCMS (ES, m/z): 573 [M+H]⁺.

### Synthesis of Intermediate F119

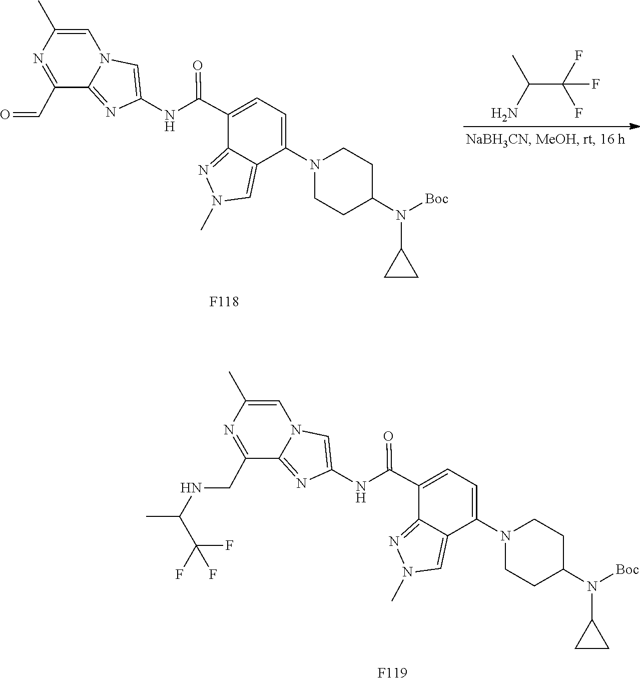

To a solution of tert-butyl N-cyclopropyl-N-[1-[7-[(8-formyl-6-methyl-imidazo[1,2-a]pyrazin-2-yl)carbamoyl]-2-methyl-indazol-4-yl]-4-piperidyl]carbamate (Intermediate F118, 60 mg, 104.77 μmol) and 1,1,1-trifluoropropan-2-amine (35.5 mg, 314.32 μmol) in MeOH (2 mL). The mixture was stirred for 1 h. Followed by the addition of NaBH₃CN (6.6 mg, 104.77 μmol), the mixture was stirred for 16 h at room temperature. The reaction mixture was quenched by water (4 mL) and extracted with EA (3×4 mL). The combined organic layers were washed with water (2×10 mL), dried over anhydrous Na₂SO₄. After filtration, the filtrate was concentrated under reduced pressure. The residue was purified by silica gel column chromatography, eluted with DCM/MeOH (10:1) to afford tert-butyl N-cyclopropyl-N-[1-[2-methyl-7-[[6-methyl-8-[[(2,2,2-trifluoro-1-methyl-ethyl)amino]methyl]imidazo[1,2-a]pyrazin-2-yl]carbamoyl]indazol-4-yl]-4-piperidyl]carbamate (Intermediate F119, 30 mg, 44.79 μmol). LCMS (ES, m/z): 670 [M+H]⁺.

### Synthesis of Compound 791

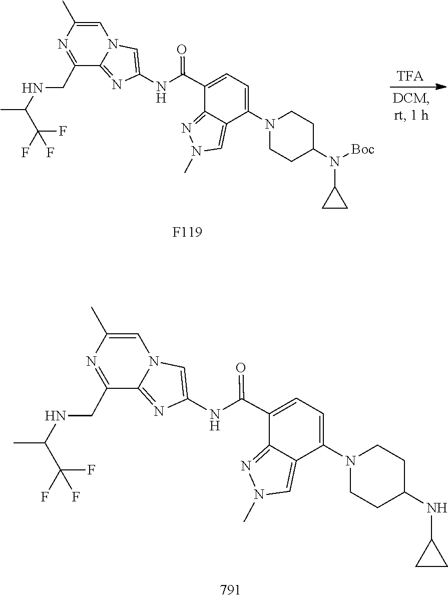 [SMILES: Cc1cn2cc(NC(=O)c3ccc(N4CCC(N(C(=O)OC(C)(C)C)C5CC5)CC4)c4cn(C)nc34)nc2c(CNC(C)C(F)(F)F)n1 ; Cc1cn2cc(NC(=O)c3ccc(N4CCC(NC5CC5)CC4)c4cn(C)nc34)nc2c(CNC(C)C(F)(F)F)n1 ; "F119"]

To a stirred mixture of tert-butyl N-cyclopropyl-N-[1-[2-methyl-7-[[6-methyl-8-[[(2,2,2-trifluoro-1-methyl-ethyl)amino]methyl]imidazo[1,2-a]pyrazin-2-yl]carbamoyl]indazol-4-yl]-4-piperidyl]carbamate (Intermediate F119, 30 mg, 44.79 μmol) in DCM (1 mL) was added TFA (0.3 mL) dropwise at room temperature. The resulting mixture was stirred for 2 h at room temperature. The resulting mixture were concentrated under reduced pressure. The crude product was purified by Prep-HPLC with the following conditions (column, C18 silica gel, XBridge, 19×150 mm; mobile phase, MeCN in water (0.05% NH₃H₂O), 30% to 80% gradient in 7 min; detector, UV 254 nm) to afford 4-[4-(cyclopropylamino)-1-piperidyl]-2-methyl-N-[6-methyl-8-[[(2,2,2-trifluoro-1-methyl-ethyl)amino]methyl]imidazo[1,2-a]pyrazin-2-yl]indazole-7-carboxamide (Compound 791, 5.3 mg, 9.30 μmol). LCMS (ES, m/z): 670 [M+H]⁺. ¹H NMR (300 MHz, DMSO-d₆) δ 11.49 (s, 1H), 8.80 (s, 1H), 8.40 (s, 1H), 8.37 (s, 1H), 8.03 (d, J=8.1 Hz, 1H), 6.51 (d, J=8.3 Hz, 1H), 4.30 (s, 5H), 3.90 (d, J=12.7 Hz, 2H), 3.54 (dd, J=12.5, 6.9 Hz, 1H), 3.09 (t, J=11.8 Hz, 2H), 2.77 (d, J=5.6 Hz, 2H), 2.43 (d, J=1.0 Hz, 3H), 2.13 (dt, J=6.5, 3.1 Hz, 1H), 2.01 (d, J=12.6 Hz, 2H), 1.48 (q, J=10.1 Hz, 2H), 1.28 (d, J=6.7 Hz, 3H), 0.40 (dt, J=6.1, 3.0 Hz, 2H), 0.29-0.19 (m, 2H).

### Example 321. Synthesis of Compound 794

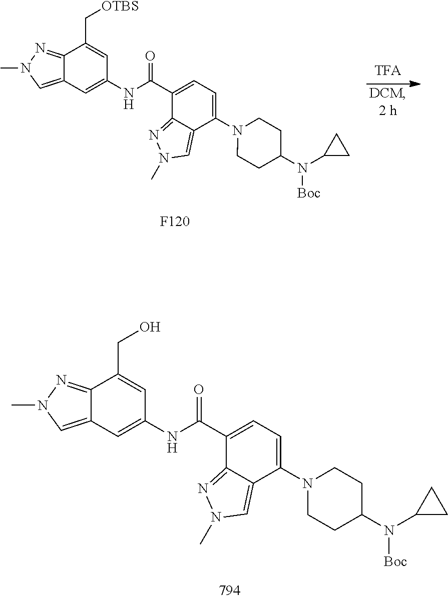 [SMILES: Cn1cc2cc(NC(=O)c3ccc(N4CCC(N(C(=O)OC(C)(C)C)C5CC5)CC4)c4cn(C)nc34)cc(CO[Si](C)(C)C(C)(C)C)c2n1 ; "DCM,2 h" ; Cn1cc2cc(NC(=O)c3ccc(N4CCC(N(C(=O)OC(C)(C)C)C5CC5)CC4)c4cn(C)nc34)cc(CO)c2n1 ; "F120" ; "TFA"]

To a stirred mixture of tert-butyl (1-(7-((7-(((tert-butyldimethylsilyl)oxy)methyl)-2-methyl-2H-indazol-5-yl)carbamoyl)-2-methyl-2H-indazol-4-yl)piperidin-4-yl) (cyclopropyl)carbamate (Intermediate F120, 50 mg, 72.68 μmol) in DCM (1 mL) was added TFA (0.2 mL) at room temperature. The resulting mixture was stirred for 2 h at rt. The mixture was concentrated under reduced pressure. The residue was purified by Prep-HPLC with the following conditions (Column: XBridge Prep OBD Column 19×150 mm 8 μm; Mobile Phase A: water (0.05% NH₃·H₂O), Mobile Phase B: ACN; Flow rate: 20 mL/min; Gradient: 50% B to 95% B in 8 min, 90% B; Wave Length: 220 nm; RT1 (min): 7.2) to afford 4-[4-(allylamino)-1-piperidyl]-N-[7-(hydroxymethyl)-2-methyl-indazol-5-yl]-2-methyl-indazole-7-carboxamide (Compound 794, 6 mg, 12.67 μmol). LCMS (ES, m/z): 474 [M+H]⁺. ¹H NMR (400 MHz, DMSO-d₆) δ 11.09 (s, 1H), 8.76 (s, 1H), 8.27 (s, 2H), 7.99 (d, J=8.1 Hz, 1H), 7.35 (s, 1H), 6.49 (d, J=8.5 Hz, 1H), 5.90 (s, 1H), 5.28 (t, J=5.8 Hz, 1H), 5.20 (d, J=16.8 Hz, 1H), 5.05 (d, J=10.5 Hz, 1H), 4.89 (d, J=5.5 Hz, 2H), 4.30 (s, 3H), 4.15 (s, 3H), 3.87 (d, J=12.9 Hz, 2H), 3.25 (d, J=5.8 Hz, 2H), 3.02 (t, J=12.2 Hz, 1H), 1.98 (d, J=12.5 Hz, 2H), 1.55-1.46 (m, 2H).

### Example 322. Synthesis of Compound 796

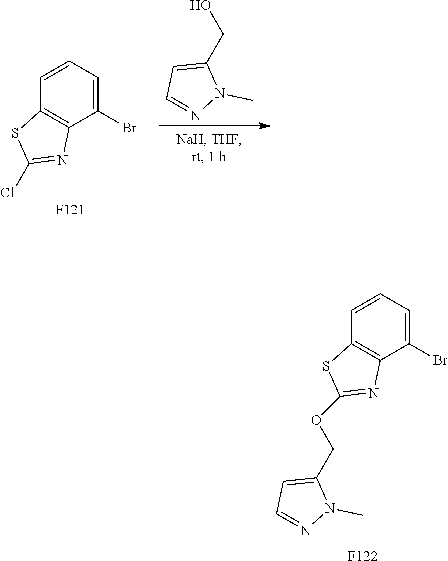

To a solution of (2-methylpyrazol-3-yl) methanol (Intermediate F121, 676.7 mg, 6.04 mmol) in THF (15 mL) was added NaH (60% dispersion in oil) (402.3 mg, 10.06 mmol) at 0° C. under N₂. The mixture was stirred for 1 h at 0° C. Then 4-bromo-2-chloro-1,3-benzothiazole (1 g, 4.02 mmol) was added at 0° C. The mixture was stirred for 1 h at rt. The reaction was diluted with H₂O (30 mL). The resulting mixture was extracted with EA (2×30 mL). The combined organic layers were washed with H₂O (2×40 mL), dried over anhydrous Na₂SO₄. After filtration, the filtrate was concentrated under reduced pressure. The residue was purified by silica gel column chromatography, eluted with PE/EA (3/1) to afford 4-bromo-2-[(2-methylpyrazol-3-yl)methoxy]-1,3-benzothiazole (Intermediate F122, 880 mg, 2.71 mmol). LCMS (ES, m/z): 324 [M+H]⁺.

### Synthesis of Intermediate F123

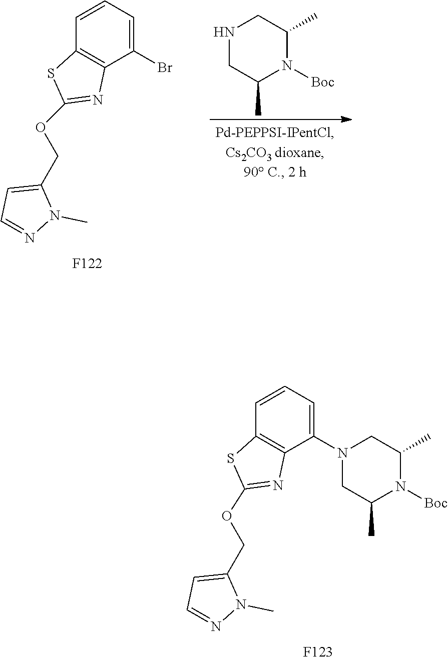

To a stirred mixture of 4-bromo-2-[(2-methylpyrazol-3-yl)methoxy]-1,3-benzothiazole (830 mg, 2.56 mmol) in dioxane (10 mL) were added tert-butyl (2S,6S)-2,6-dimethylpiperazine-1-carboxylate (Intermediate F122, 658.4 mg, 3.07 mmol), Cs₂CO₃ (1.67 g, 5.12 mmol) and Pd-PEPPSI-IPentCl (215.1 mg, 256.02 μmol) at rt under N₂. The mixture was stirred for 2 h at 90° C. under N₂. The reaction was diluted with H₂O (25 mL). The resulting mixture was extracted with DCM (2×25 mL). The combined organic layers were washed with H₂O (30 mL), dried over anhydrous Na₂SO₄. After filtration, the filtrate was concentrated under reduced pressure. The residue was purified by silica gel column chromatography, eluted with PE/EA (2/1) to afford tert-butyl (2S,6S)-2,6-dimethyl-4-[2-[(2-methylpyrazol-3-yl)methoxy]-1,3-benzothiazol-4-yl]piperazine-1-carboxylate (Intermediate F123, 370 mg, 808.59 μmol). LCMS (ES, m/z): 458 [M+H]⁺.

### Synthesis of Intermediate F124

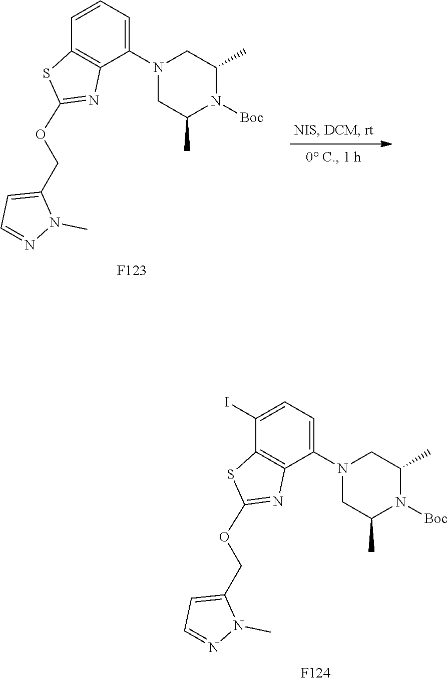

To a solution of tert-butyl (2S,6S)-2,6-dimethyl-4-[2-[(2-methylpyrazol-3-yl)methoxy]-1,3-benzothiazol-4-yl]piperazine-1-carboxylate (Intermediate F123, 340 mg, 743.03 μmol) in DCM (4 mL) was added NIS (167.1 mg, 743.03 μmol) at 0° C. The mixture was stirred for 1 h at 0° C. The reaction was diluted with H₂O (20 mL). The resulting mixture was extracted with DCM (2×20 mL). The combined organic layers were washed with H₂O (30 mL), dried over anhydrous Na₂SO₄. After filtration, the filtrate was concentrated under reduced pressure. The residue was purified by silica gel column chromatography, eluted with PE/EA (2/1) to afford tert-butyl (2S,6S)-4-[7-iodo-2-[(2-methylpyrazol-3-yl)methoxy]-1,3-benzothiazol-4-yl]-2,6-dimethyl-piperazine-1-carboxylate (Intermediate F124, 240 mg, 411.32 μmol). LCMS (ES, m/z): 584 [M+H]⁺.

### Synthesis of Intermediate F125

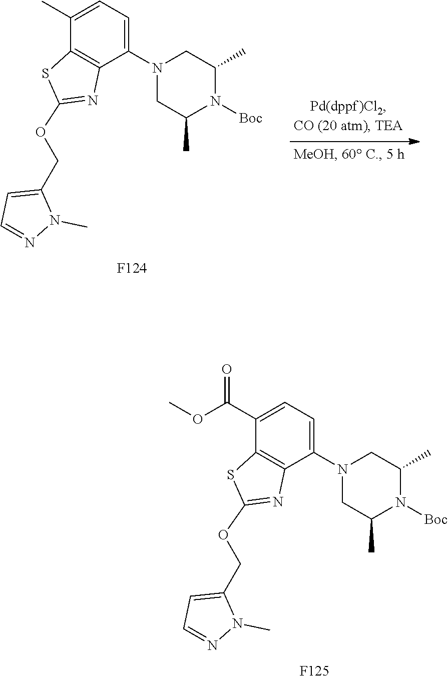

To a solution of tert-butyl (2S,6S)-4-[7-iodo-2-[(2-methylpyrazol-3-yl)methoxy]-1,3-benzothiazol-4-yl]-2,6-dimethyl-piperazine-1-carboxylate (Intermediate F124, 240 mg, 411.32 μmol) in MeOH (30 mL) were added TEA (41.6 mg, 411.32 μmol) and Pd(dppf)Cl₂·CH₂Cl₂ (33.6 mg, 41.13 μmol) in a pressure tank. The mixture was pressurized to 20 atm with carbon monoxide at 60° C. for 5 h. The reaction mixture was cooled to room temperature and filtered to remove insoluble solids. The resulting mixture was concentrated under reduced pressure. The residue was purified by silica gel column chromatography, eluted with PE/EA (3/1) to afford methyl 4-[(3S,5S)-4-tert-butoxycarbonyl-3,5-dimethyl-piperazin-1-yl]-2-[(2-methylpyrazol-3-yl)methoxy]-1,3-benzothiazole-7-carboxylate (Intermediate F125, 110 mg, 213.33 μmol). LCMS (ES, m/z): 516 [M+H]⁺.

### Synthesis of Intermediate F126

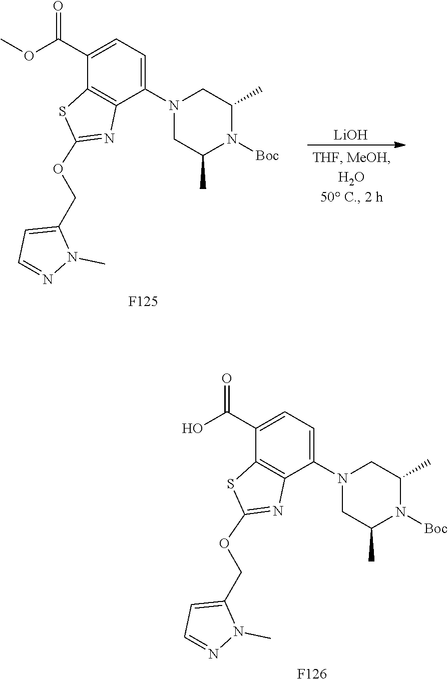

To a solution of methyl 4-[(3S,5S)-4-tert-butoxycarbonyl-3,5-dimethyl-piperazin-1-yl]-2-[(2-methylpyrazol-3-yl)methoxy]-1,3-benzothiazole-7-carboxylate (Intermediate F125, 110 mg, 213.33 μmol) in THF (1 mL)/MeOH (0.5 mL)/H₂O (0.5 mL) was added LiOH·H₂O (44.7 mg, 1.07 mmol) at rt. The mixture was stirred for 2 h at 50° C. The resulting mixture was diluted with H₂O and adjusted to pH 5-6 with 1N HCl. The resulting mixture was extracted with DCM (2×15 mL). The combined organic layers were washed with H₂O (2×15 mL), dried over anhydrous Na₂SO₄. The resulting mixture was concentrated under reduced pressure to afford 4-[(3S,5S)-4-tert-butoxycarbonyl-3,5-dimethyl-piperazin-1-yl]-2-[(2-methylpyrazol-3-yl)methoxy]-1,3-benzothiazole-7-carboxylic acid (Intermediate F126, 100 mg, 199.36 μmol). LCMS (ES, m/z): 502 [M+H]⁺.

### Synthesis of Intermediate F127

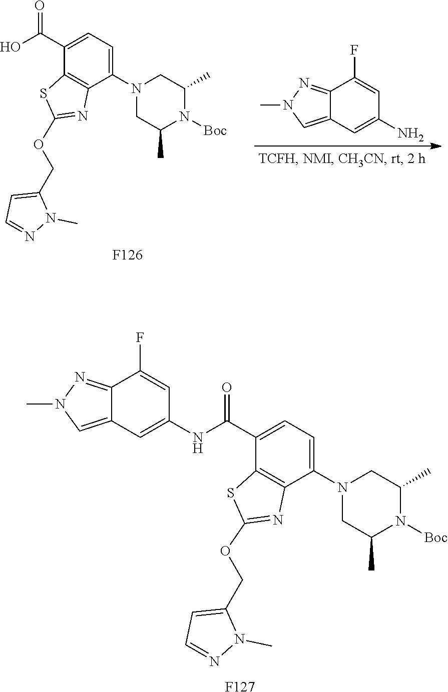

To a solution of 4-[(3S,5S)-4-tert-butoxycarbonyl-3,5-dimethyl-piperazin-1-yl]-2-[(2-methylpyrazol-3-yl)methoxy]-1,3-benzothiazole-7-carboxylic acid (Intermediate F126, 90 mg, 179.43 μmol) in MeCN (1 mL) were added 7-fluoro-2-methyl-indazol-5-amine (35.5 mg, 215.31 μmol), NMI (58.9 mg, 717.71 μmol) and TCFH (75.5 mg, 269.14 μmol). The mixture was stirred for 2 h at rt. The reaction was diluted with H₂O (15 mL). The resulting mixture was extracted with DCM (2×15 mL). The combined organic layers were washed with H₂O (15 mL), dried over anhydrous Na₂SO₄. After filtration, the filtrate was concentrated under reduced pressure. The residue was purified by silica gel column chromatography, eluted with PE/EA (1/4) to afford tert-butyl (2S,6S)-4-[7-[(7-fluoro-2-methyl-indazol-5-yl)carbamoyl]-2-[(2-methylpyrazol-3-yl)methoxy]-1,3-benzothiazol-4-yl]-2,6-dimethyl-piperazine-1-carboxylate (Intermediate F127, 90 mg, 138.73 μmol). LCMS (ES, m/z): 649 [M+H]⁺.

### Synthesis of Compound 796

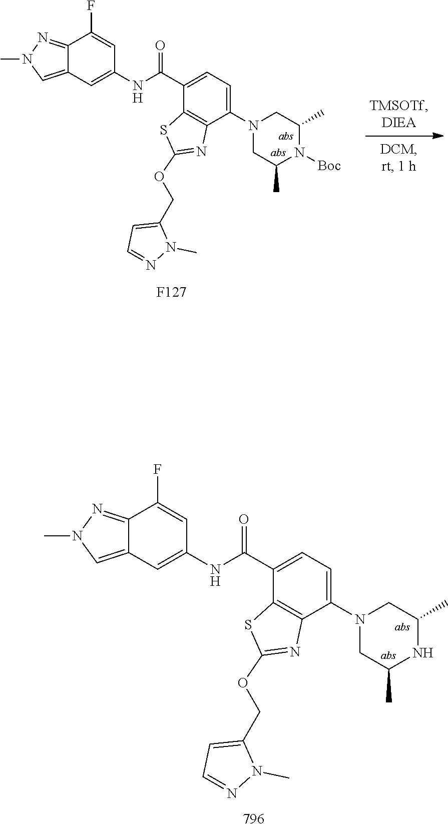

To a solution of tert-butyl (2S,6S)-4-[7-[(7-fluoro-2-methyl-indazol-5-yl)carbamoyl]-2-[(2-methylpyrazol-3-yl)methoxy]-1,3-benzothiazol-4-yl]-2,6-dimethyl-piperazine-1-carboxylate (Intermediate F127, 90 mg, 138.73 μmol) in DCM (1.5 mL) were added DIEA (35.8 mg, 277.46 μmol) and TMSOTf (92.5 mg, 416.19 μmol). The mixture was stirred for 1 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 40% to 55% gradient in 7 min; detector, UV 254 nm) to afford 4-[(3S,5S)-3,5-dimethylpiperazin-1-yl]-N-(7-fluoro-2-methyl-indazol-5-yl)-2-[(2-methylpyrazol-3-yl)methoxy]-1,3-benzothiazole-7-carboxamide (Compound 796, 22 mg, 40.10 μmol). LCMS (ES, m/z): 549 [M+H]⁺. ¹H NMR (400 MHz, DMSO-d₆) δ 10.25 (s, 1H), 8.43 (d, J=2.7 Hz, 1H), 8.12 (d, J=8.5 Hz, 1H), 8.06 (s, 1H), 7.46-7.38 (m, 2H), 6.98 (d, J=8.6 Hz, 1H), 6.47 (d, J=1.9 Hz, 1H), 5.75-5.62 (m, 2H), 4.19 (s, 3H), 3.91 (s, 3H), 3.38 (td, J=12.2, 11.7, 4.6 Hz, 3H), 3.27 (td, J=12.3, 6.9 Hz, 3H), 2.06 (s, 1H), 1.20 (d, J=6.2 Hz, 6H).

### Example 323. Synthesis of Compound 797

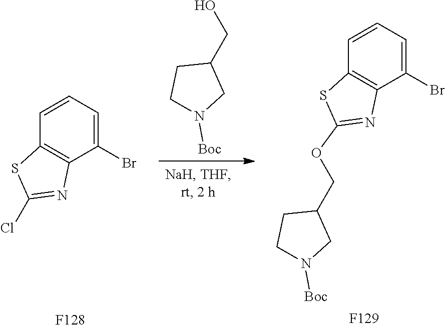

A solution of tert-butyl 3-(hydroxymethyl)pyrrolidine-1-carboxylate (1.21 g, 6.04 mmol) in THF (15 mL) was treated with NaH (60% dispersion in oil) (193.1 mg, 8.05 mmol) for 30 min at 0° C. under nitrogen atmosphere followed by the addition of 4-bromo-2-chloro-1,3-benzothiazole (Intermediate F128, 1 g, 4.02 mmol) in portions at 0° C. The resulting mixture was stirred for 2 h at room temperature. The reaction was quenched with sat. NH₄Cl (aq.) at 0° C. The resulting mixture was extracted with EA (2×30 mL). The combined organic layers were washed with brine (1×40 mL), dried over anhydrous Na₂SO₄. After filtration, the filtrate was concentrated under reduced pressure. This resulted in tert-butyl 3-[(4-bromo-1,3-benzothiazol-2-yl)oxymethyl]pyrrolidine-1-carboxylate (Intermediate F129, 1.05 g, 2.54 mmol). LCMS (ES, m/z): 413 [M+H]⁺.

### Synthesis of Intermediate F130

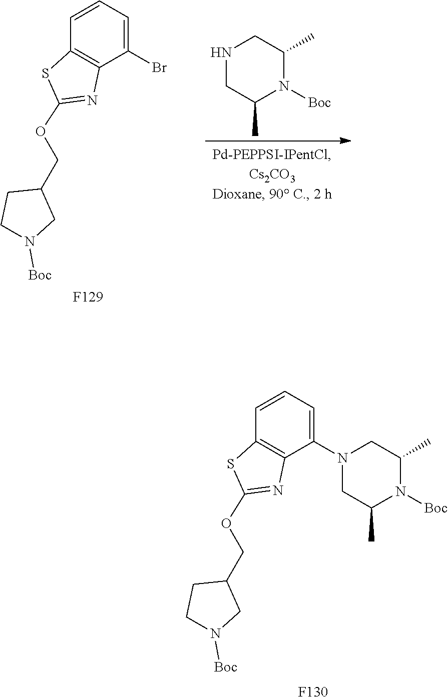

To a stirred mixture of tert-butyl 3-[(4-bromo-1,3-benzothiazol-2-yl)oxymethyl]pyrrolidine-1-carboxylate (Intermediate F129, 650 mg, 1.57 mmol) and tert-butyl (2S,6S)-2,6-dimethylpiperazine-1-carboxylate (438.1 mg, 2.04 mmol) in Dioxane (13 mL) were added Cs₂CO₃ (1.54 g, 4.72 mmol), Pd-PEPPSI-IPentCl (132.1 mg, 157.26 μmol) at room temperature under nitrogen atmosphere. The resulting mixture was stirred for 2 h at 90° C. under nitrogen atmosphere. The mixture was allowed to cool down to room temperature. The resulting mixture was purified by silica gel column chromatography, eluted with PE/EA (5:1) to afford tert-butyl (2S,6S)-4-[2-[(1-tert-butoxycarbonylpyrrolidin-3-yl)methoxy]-1,3-benzothiazol-4-yl]-2,6-dimethyl-piperazine-1-carboxylate (Intermediate F130, 700 mg, 1.28 mmol). LCMS (ES, m/z): 547 [M+H]⁺.

### Synthesis of Intermediate F131

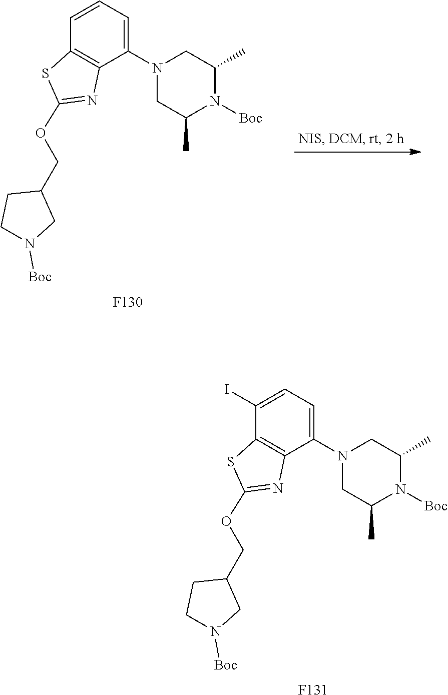 [SMILES: C[C@H]1CN(c2cccc3sc(OCC4CCN(C(=O)OC(C)(C)C)C4)nc23)C[C@H](C)N1C(=O)OC(C)(C)C ; "F130" ; C[C@H]1CN(c2ccc(I)c3sc(OCC4CCN(C(=O)OC(C)(C)C)C4)nc23)C[C@H](C)N1C(=O)OC(C)(C)C ; "F131"]

A solution of tert-butyl (2S,6S)-4-[2-[(1-tert-butoxycarbonylpyrrolidin-3-yl)methoxy]-1,3-benzothiazol-4-yl]-2,6-dimethyl-piperazine-1-carboxylate (Intermediate F130, 700 mg, 1.28 mmol) in DCM (7 mL) was treated with NIS (288 mg, 1.28 mmol) at 0° C. under nitrogen atmosphere. The resulting mixture was stirred for 2 h at room temperature under nitrogen atmosphere. The resulting mixture was diluted with water (20 mL). The resulting mixture was extracted with EA (2×40 mL). The combined organic layers were washed with brine (1×20 mL), dried over anhydrous Na₂SO₄. After filtration, the filtrate was concentrated under reduced pressure to afford tert-butyl (2S,6S)-4-[2-[(1-tert-butoxycarbonylpyrrolidin-3-yl)methoxy]-7-iodo-1,3-benzothiazol-4-yl]-2,6-dimethyl-piperazine-1-carboxylate (Intermediate F131, 705 mg, 1.04 mmol). LCMS (ES, m/z): 673 [M+H]⁺.

### Synthesis of Intermediate F132

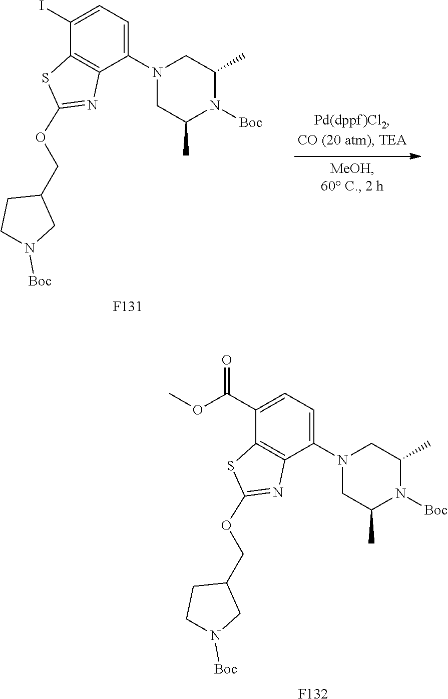 [SMILES: C[C@H]1CN(c2ccc(I)c3sc(OCC4CCN(C(=O)OC(C)(C)C)C4)nc23)C[C@H](C)N1C(=O)OC(C)(C)C ; "F131" ; COC(=O)c1ccc(N2C[C@H](C)N(C(=O)OC(C)(C)C)[C@@H](C)C2)c2nc(OCC3CCN(C(=O)OC(C)(C)C)C3)sc12 ; "F132"]

To a solution of tert-butyl (2S,6S)-4-[2-[(1-tert-butoxycarbonylpyrrolidin-3-yl)methoxy]-7-iodo-1,3-benzothiazol-4-yl]-2,6-dimethyl-piperazine-1-carboxylate (Intermediate F131, 620 mg, 921.77 μmol) and TEA (279.8 mg, 2.77 mmol) in MeOH (10 mL) was added Pd(dppf)Cl₂ (67.4 mg, 92.18 μmol) in a pressure tank. The mixture was purged with nitrogen and then was pressurized to 20 atm with carbon monoxide at 60° C. for 2 h. The reaction mixture was cooled to room temperature and filtered to remove insoluble solids. The resulting mixture was concentrated under reduced pressure to afford methyl 4-[(3S,5S)-4-tert-butoxycarbonyl-3,5-dimethyl-piperazin-1-yl]-2-[(1-tert-butoxycarbonylpyrrolidin-3-yl)methoxy]-1,3-benzothiazole-7-carboxylate (Intermediate F132, 480 mg, 793.71 μmol). LCMS (ES, m/z): 605 [M+H]⁺.

### Synthesis of Intermediate F133

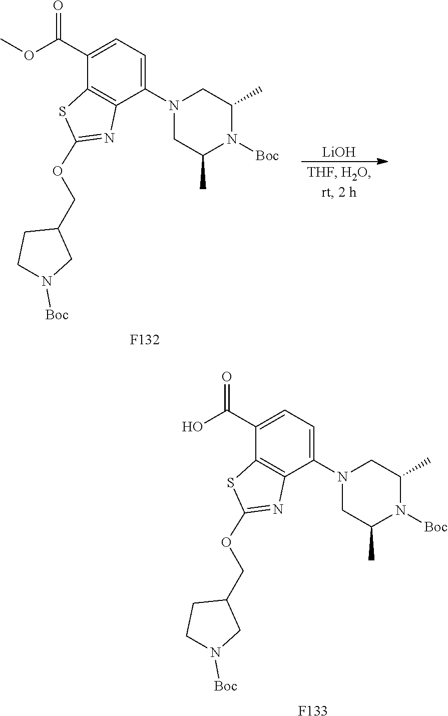 [SMILES: COC(=O)c1ccc(N2C[C@H](C)N(C(=O)OC(C)(C)C)[C@@H](C)C2)c2nc(OCC3CCN(C(=O)OC(C)(C)C)C3)sc12 ; "LiOH" ; "THF, H2O,rt, 2 h" ; "F132" ; C[C@H]1CN(c2ccc(C(=O)O)c3sc(OCC4CCN(C(=O)OC(C)(C)C)C4)nc23)C[C@H](C)N1C(=O)OC(C)(C)C ; "F133"]

A solution of methyl 4-[(3S,5S)-4-tert-butoxycarbonyl-3,5-dimethyl-piperazin-1-yl]-2-[(1-tert-butoxycarbonylpyrrolidin-3-yl)methoxy]-1,3-benzothiazole-7-carboxylate (Intermediate F132, 260 mg, 429.93 μmol) in THF (2 mL) and H₂O (2 mL) was treated with LiOH·H₂O (98%) (90.2 mg, 2.15 mmol) at room temperature. The resulting mixture was stirred for 2 h at rt under nitrogen atmosphere. The mixture was acidified to pH 4 with HCl (1N). The resulting mixture was extracted with EA (3×20 mL). The combined organic layers were washed with brine (1×30 mL), dried over anhydrous Na₂SO₄. After filtration, the filtrate was concentrated under reduced pressure. This resulted in 4-[(3S,5S)-4-tert-butoxycarbonyl-3,5-dimethyl-piperazin-1-yl]-2-[(1-tert-butoxycarbonylpyrrolidin-3-yl)methoxy]-1,3-benzothiazole-7-carboxylic acid (Intermediate F133, 210 mg, 355.49 μmol). LCMS (ES, m/z): 591 [M+H]⁺.

### Synthesis of Intermediate F134

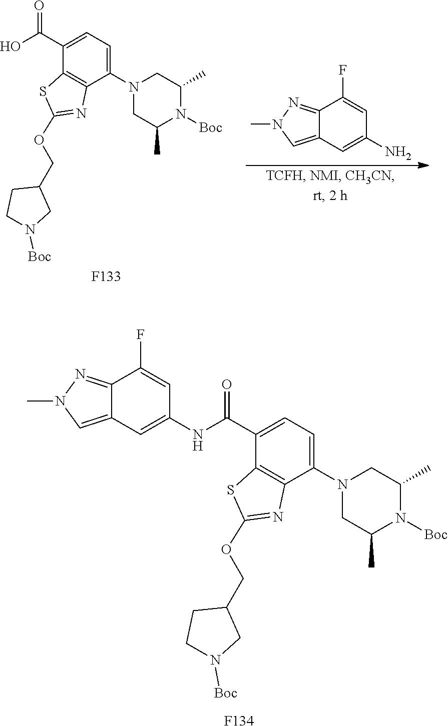

To a stirred mixture of 4-[(3S,5S)-4-tert-butoxycarbonyl-3,5-dimethyl-piperazin-1-yl]-2-[(1-tert-butoxycarbonylpyrrolidin-3-yl)methoxy]-1,3-benzothiazole-7-carboxylic acid (Intermediate F133, 70 mg, 118.50 μmol) and 7-fluoro-2-methyl-indazol-5-amine (23.49 mg, 142.20 μmol) in CH₃CN (2 mL) were added NMI (29.1 mg, 355.49 μmol) and TCFH (49.8 mg, 177.75 μmol) at room temperature. The resulting mixture was stirred for 2 h at rt. The resulting mixture was diluted with water (10 mL). The resulting mixture was extracted with EA (3×15 mL). The combined organic layers were washed with brine (1×30 mL), dried over anhydrous Na₂SO₄. After filtration, the filtrate was concentrated under reduced pressure. The residue was purified by silica gel column chromatography, eluted with EA to afford tert-butyl (2S,6S)-4-[2-[(1-tert-butoxycarbonylpyrrolidin-3-yl)methoxy]-7-[(7-fluoro-2-methyl-indazol-5-yl)carbamoyl]-1,3-benzothiazol-4-yl]-2,6-dimethyl-piperazine-1-carboxylate (Intermediate F134, 80 mg, 108.42 μmol). LCMS (ES, m/z): 738 [M+H]⁺.

### Synthesis of Compound 797

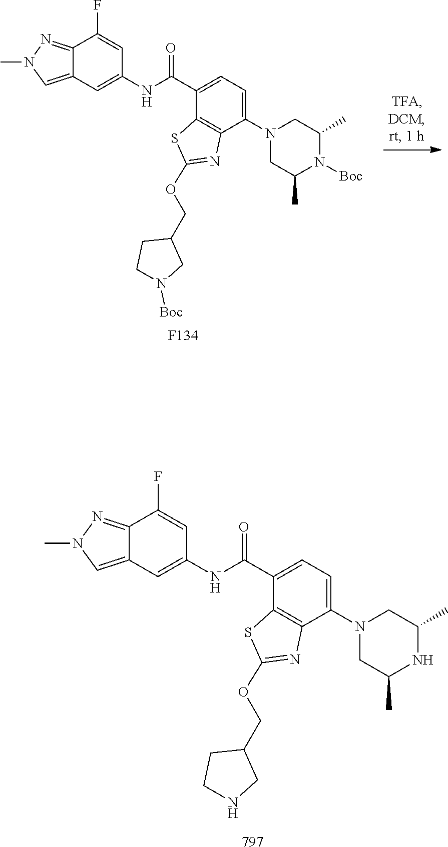

To a stirred mixture of tert-butyl (2S,6S)-4-[2-[(1-tert-butoxycarbonylpyrrolidin-3-yl)methoxy]-7-[(7-fluoro-2-methyl-indazol-5-yl)carbamoyl]-1,3-benzothiazol-4-yl]-2,6-dimethyl-piperazine-1-carboxylate (Intermediate F134, 80 mg, 108.42 μmol) in DCM (2 mL) was added TFA (1 mL) dropwise at room temperature. The resulting mixture was stirred for 1 h at room temperature. The resulting mixture was concentrated under reduced pressure. The crude product was purified by Prep-HPLC with the following conditions (column, C18 silica gel, XBridge, 19×150 mm; mobile phase, MeCN in water (0.05% NH₃·H₂O), 40% to 85% gradient in 7 min; detector, UV 254 nm) to afford 4-((3S,5S)-3,5-dimethylpiperazin-1-yl)-N-(7-fluoro-2-methyl-2H-indazol-5-yl)-2-(pyrrolidin-3-ylmethoxy)benzo[d]thiazole-7-carboxamide bis(2,2,2-trifluoroacetate) (Compound 797, 32 mg, 41.79 μmol). LCMS (ES, m/z): 538 [M+H]⁺. ¹H NMR (300 MHz, DMSO-d₆) δ 10.34 (s, 1H), 9.03 (s, 2H), 8.91 (s, 2H), 8.45 (d, J=2.8 Hz, 1H), 8.18 (d, J=8.6 Hz, 1H), 8.04 (d, J=1.6 Hz, 1H), 7.43 (dd, J=13.5, 1.6 Hz, 1H), 7.11 (d, J=8.6 Hz, 1H), 4.73-4.45 (m, 2H), 4.20 (s, 3H), 3.85-3.64 (m, 4H), 3.57 (d, J=11.7 Hz, 2H), 3.42 (s, 1H), 3.36-3.15 (m, 2H), 3.16-3.00 (m, 1H), 2.89 (p, J=7.3 Hz, 1H), 2.24-2.07 (m, 1H), 1.89-1.71 (m, 1H), 1.44 (d, J=6.1 Hz, 6H).

### Example 324. Synthesis of Compound 798

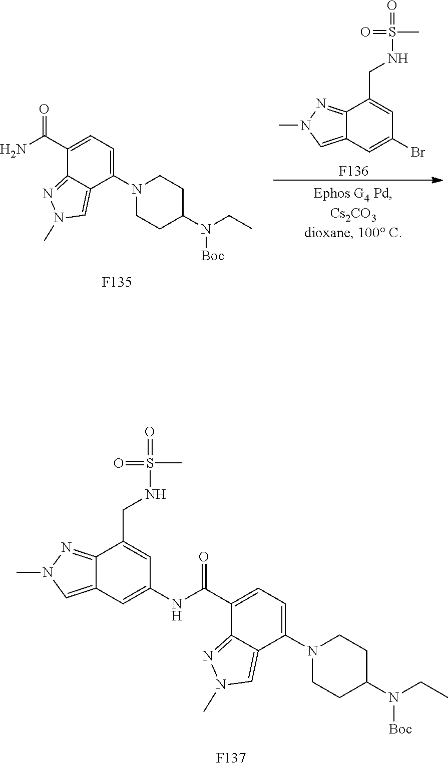

To a stirred mixture of tert-butyl N-[1-(7-carbamoyl-2-methyl-indazol-4-yl)-4-piperidyl]-N-ethyl-carbamate (Intermediate F135, 50 mg, 124.53 μmol) and N-[(5-bromo-2-methyl-indazol-7-yl)methyl]methanesulfonamide (Intermediate F136, 47.5 mg, 149.44 μmol) in dioxane (1 mL) were added Ephos G4 Pd (11.4 mg, 12.45 μmol) and Cs₂CO₃ (81.1 mg, 249.07 μmol) at room temperature under nitrogen atmosphere. The resulting mixture was stirred for 2 h at 100° C. under nitrogen atmosphere. The resulting mixture was concentrated under reduced pressure. The residue was purified by silica gel column chromatography, eluted with PE/EA (1:3) to afford tert-butyl N-ethyl-N-[1-[7-[[7-(methanesulfonamidomethyl)-2-methyl-indazol-5-yl]carbamoyl]-2-methyl-indazol-4-yl]-4-piperidyl]carbamate (Intermediate F138, 45 mg, 70.45 μmol). LCMS (ES, m/z): 639 [M+H]⁺.

### Synthesis of Compound 798

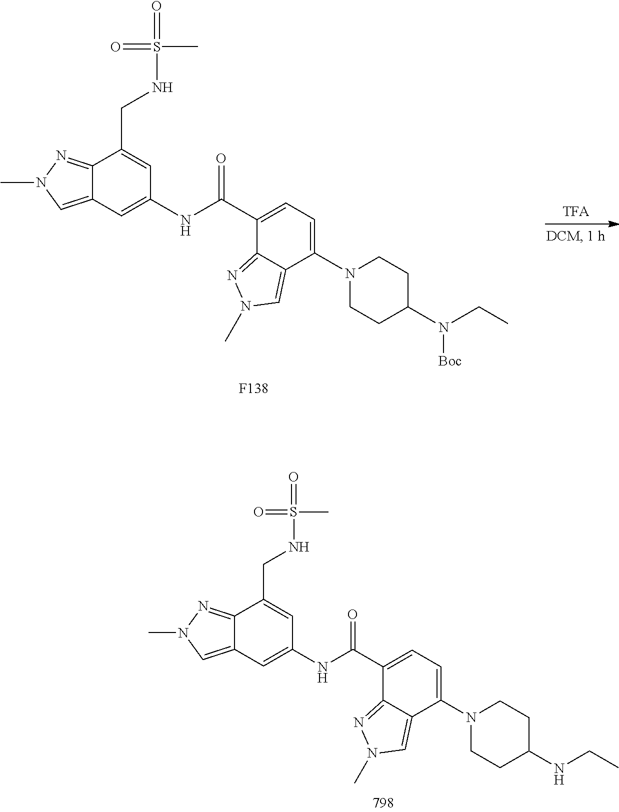

To a stirred solution of tert-butyl N-ethyl-N-[1-[7-[[7-(methanesulfonamidomethyl)-2-methyl-indazol-5-yl]carbamoyl]-2-methyl-indazol-4-yl]-4-piperidyl]carbamate (Intermediate F138, 45 mg, 70.45 μmol) in DCM (2 mL) was added TFA (1 mL) at rt. The resulting mixture was stirred for 1 h. The resulting mixture was concentrated under reduced pressure. The crude product was purified by Prep-HPLC with the following conditions (SunFire Prep C18 OBD Column 19*150 mm, 5 Um 10 nm, mobile phase, MeCN in water (0.05% NH₄OH), 35% to 65% gradient in 7 min; detector, UV 254 nm) to afford 4-[4-(ethylamino)-1-piperidyl]-N-[7-(methanesulfonamidomethyl)-2-methyl-indazol-5-yl]-2-methyl-indazole-7-carboxamide (Compound 798, 10 mg, 18.56 μmol). LCMS (ES, m/z): 539 [M+H]⁺. ¹H NMR (400 MHz, DMSO-d₆) δ 11.14 (s, 1H), 8.76 (s, 1H), 8.32 (s, 1H), 8.25 (d, J=1.9 Hz, 1H), 7.99 (d, J=8.0 Hz, 1H), 7.62 (s, 1H), 7.43 (s, 1H), 6.49 (d, J=8.2 Hz, 1H), 4.55 (s, 2H), 4.30 (s, 3H), 4.18 (s, 3H), 3.87 (d, J=12.7 Hz, 2H), 3.05 (d, J=11.8 Hz, 2H), 3.01 (s, 3H), 2.65 (s, 1H), 2.62 (q, J=7.2 Hz, 2H), 1.97 (d, J=12.6 Hz, 2H), 1.45 (q, J=11.0 Hz, 2H), 1.05 (t, J=7.1 Hz, 3H).

### Example 325. Synthesis of Compound 800

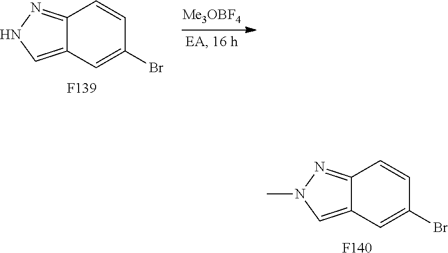

To a stirred solution of 5-bromo-2H-indazole (Intermediate F139, 2 g, 10.15 mmol) in EA (40 mL) was added Me₃OBF₄ (2.25 g, 15.23 mmol) at rt. The resulting mixture was stirred for 16 h. The mixture was diluted with water (40 mL) and extracted with EA (2×40 mL). The combined organic layer was washed with water (2×50 mL) and dried by anhydrous Na₂SO₄. The resulting organic layer was concentrated under reduced pressure to afford 5-bromo-2-methyl-indazole (Intermediate F140, 1.9 g, 9.00 mmol). LCMS (ES, m/z): 211 [M+H]⁺.

### Synthesis of Intermediate F142

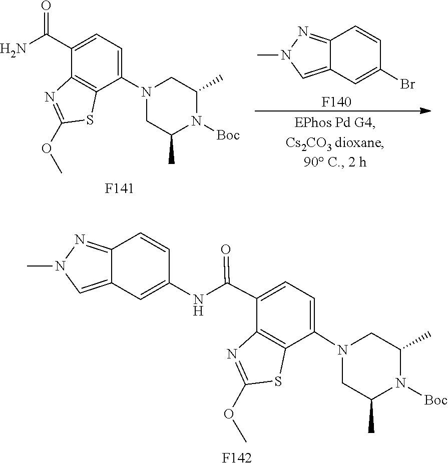

To a stirred mixture of tert-butyl (2S,6S)-4-(4-carbamoyl-2-methoxy-1,3-benzothiazol-7-yl)-2,6-dimethyl-piperazine-1-carboxylate (Intermediate F141, 50 mg, 118.90 μmol) and 5-bromo-2-methyl-indazole (Intermediate F140, 25.0 mg, 118.90 μmol) in dioxane (1 mL) were added Cs₂CO₃ (38.7 mg, 118.90 μmol) and EPhos Pd G4 (10.9 mg, 11.89 μmol) at room temperature under nitrogen atmosphere. The resulting mixture was stirred for 2 h at 90° C. under nitrogen atmosphere. The resulting mixture was concentrated under reduced pressure. The residue was purified by silica gel column chromatography, eluted with PE/EA (1:3) to afford tert-butyl (2S,6S)-4-[2-methoxy-4-[(2-methylindazol-5-yl)carbamoyl]-1,3-benzothiazol-7-yl]-2,6-dimethyl-piperazine-1-carboxylate (Intermediate F142, 55 mg, 99.88 μmol). LCMS (ES, m/z): 551 [M+H]⁺.

### Synthesis of Compound 800

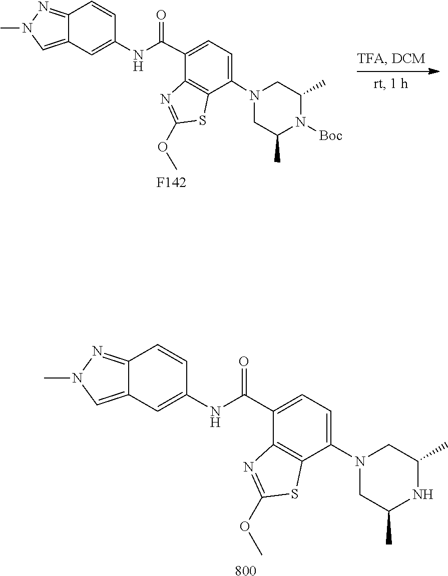

To a stirred solution of tert-butyl (2S,6S)-4-[2-methoxy-4-[(2-methylindazol-5-yl)carbamoyl]-1,3-benzothiazol-7-yl]-2,6-dimethyl-piperazine-1-carboxylate (Intermediate F142, 55 mg, 99.88 μmol) in DCM (2 mL) was added TFA (1.49 g, 13.07 mmol, 1 mL) at rt. The resulting mixture was stirred for 2 h. The resulting mixture was concentrated under reduced pressure. The crude product was purified by Prep-HPLC with the following conditions (SunFire Prep C18 OBD Column 19*150 mm, 5 Um 10 nm, mobile phase, MeCN in water (0.05% NH₄OH), 50% to 60% gradient in 7 min; detector, UV 254 nm) to afford 7-[(3S,5S)-3,5-dimethylpiperazin-1-yl]-2-methoxy-N-(2-methylindazol-5-yl)-1,3-benzothiazole-4-carboxamide (Compound 800, 17.3 mg, 38.40 μmol). LCMS (ES, m/z): 451 [M+H]⁺.

### Example 326. Synthesis of Compound 801

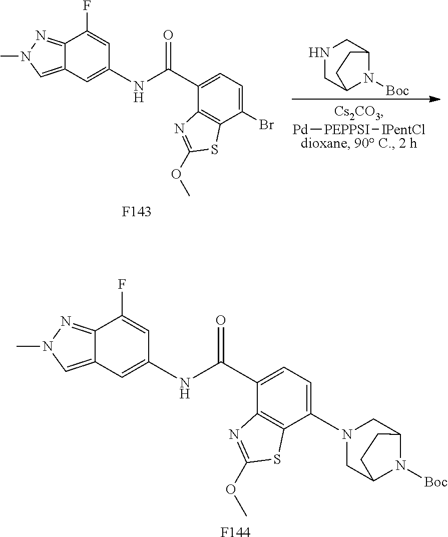

To a stirred mixture of 7-bromo-N-(7-fluoro-2-methyl-indazol-5-yl)-2-methoxy-1,3-benzothiazole-4-carboxamide (Intermediate F143, 50 mg, 114.87 μmol) in dioxane (1 mL) were added tert-butyl 3,8-diazabicyclo[3.2.1]octane-8-carboxylate (29.2 mg, 137.85 μmol), Cs₂CO₃ (74.8 mg, 229.74 μmol) and Pd-PEPPSI-IPentCl (9.6 mg, 11.49 μmol) at rt under N₂. The mixture was stirred for 2 h at 90° C. under N₂. The reaction was diluted with H₂O (10 mL). The resulting mixture was extracted with DCM (2×10 mL). The combined organic layers were washed with H₂O (10 mL), dried over anhydrous Na₂SO₄. After filtration, the filtrate was concentrated under reduced pressure. The residue was purified by silica gel column chromatography, eluted with PE/EA (1/1) to afford tert-butyl 3-[4-[(7-fluoro-2-methyl-indazol-5-yl)carbamoyl]-2-methoxy-1,3-benzothiazol-7-yl]-3,8-diazabicyclo[3.2.1]octane-8-carboxylate (Intermediate F144, 42 mg, 74.12 μmol). LCMS (ES, m/z): 567 [M+H]⁺.

### Synthesis of Compound 801

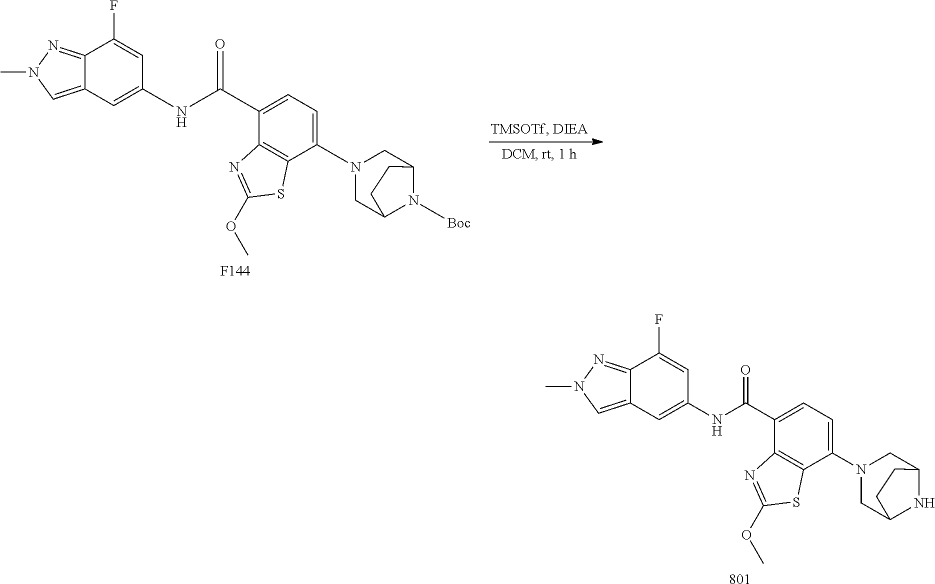

To a solution of tert-butyl 3-[4-[(7-fluoro-2-methyl-indazol-5-yl)carbamoyl]-2-methoxy-1,3-benzothiazol-7-yl]-3,8-diazabicyclo[3.2.1]octane-8-carboxylate (Intermediate F144, 40 mg, 70.59 μmol) in DCM (0.5 mL) were added DIEA (27.3 mg, 211.77 μmol) and TMSOTf (47.0 mg, 211.77 μmol). The mixture was stirred for 1 h at rt. The resulting mixture was concentrated under reduced pressure. The crude product was purified by Prep-HPLC with the following conditions (column, C18 silica gel, XBridge, 19×150 mm; mobile phase, MeCN in water (0.05% NH₃H₂O), 45% to 65% gradient in 7 min; detector, UV 254 nm) to afford 7-(3,8-diazabicyclo[3.2.1]octan-3-yl)-N-(7-fluoro-2-methyl-indazol-5-yl)-2-methoxy-1,3-benzothiazole-4-carboxamide (Compound 801, 15 mg, 32.15 μmol). LCMS (ES, m/z): 467 [M+H]⁺. ¹H NMR (400 MHz, DMSO-d₆) δ 11.39 (s, 1H), 8.40 (d, J=2.8 Hz, 1H), 8.11-8.04 (m, 2H), 7.33 (d, J=13.5 Hz, 1H), 6.96 (d, J=8.7 Hz, 1H), 4.38 (s, 3H), 4.17 (s, 3H), 3.50 (s, 2H), 3.36-3.29 (m, 2H), 2.98 (d, J=10.8 Hz, 2H), 1.81 (d, J=7.0 Hz, 2H), 1.76-1.64 (m, 2H).

### Example 327. Synthesis of Compound 804

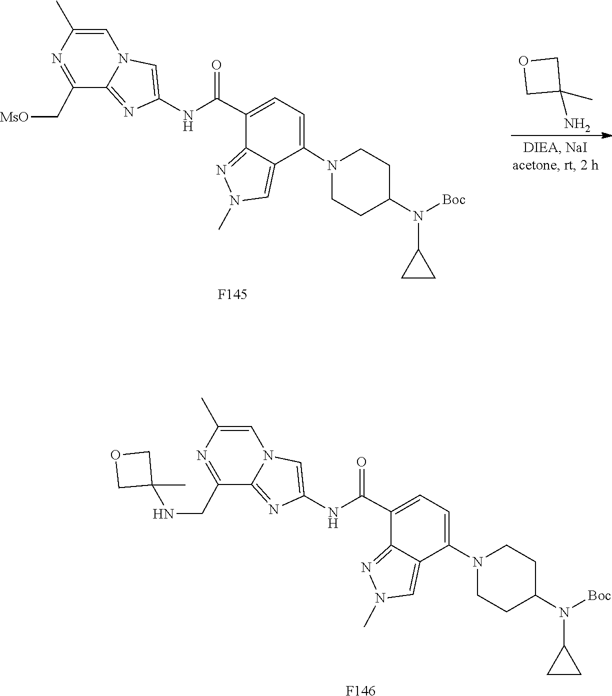

To a stirred solution of [2-[[4-[4-[tert-butoxycarbonyl(cyclopropyl)amino]-1-piperidyl]-2-methyl-indazole-7-carbonyl]amino]-6-methyl-imidazo[1,2-a]pyrazin-8-yl]methyl methanesulfonate (Intermediate F145, 100 mg, 153.20 μmol) and 3-methyloxetan-3-amine hydrochloride (37.8 mg, 306.39 μmol) in acetone (1 mL) was added NaI (45.9 mg, 306.39 μmol) and DIEA (59.4 mg, 459.59 μmol) at 0° C. The resulting mixture was stirred for 2 h at rt. The resulting mixture was diluted with water (6 mL). The resulting mixture was extracted with EA (3×5 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (1:2) to afford tert-butyl N-cyclopropyl-N-[1-[2-methyl-7-[[6-methyl-8-[[(3-methyloxetan-3-yl)amino]methyl]imidazo[1,2-a]pyrazin-2-yl]carbamoyl]indazol-4-yl]-4-piperidyl]carbamate (Intermediate F146, 60 mg, 37.28 μmol). LCMS (ES, m/z): 644 [M+H]⁺.

### Synthesis of Compound 804

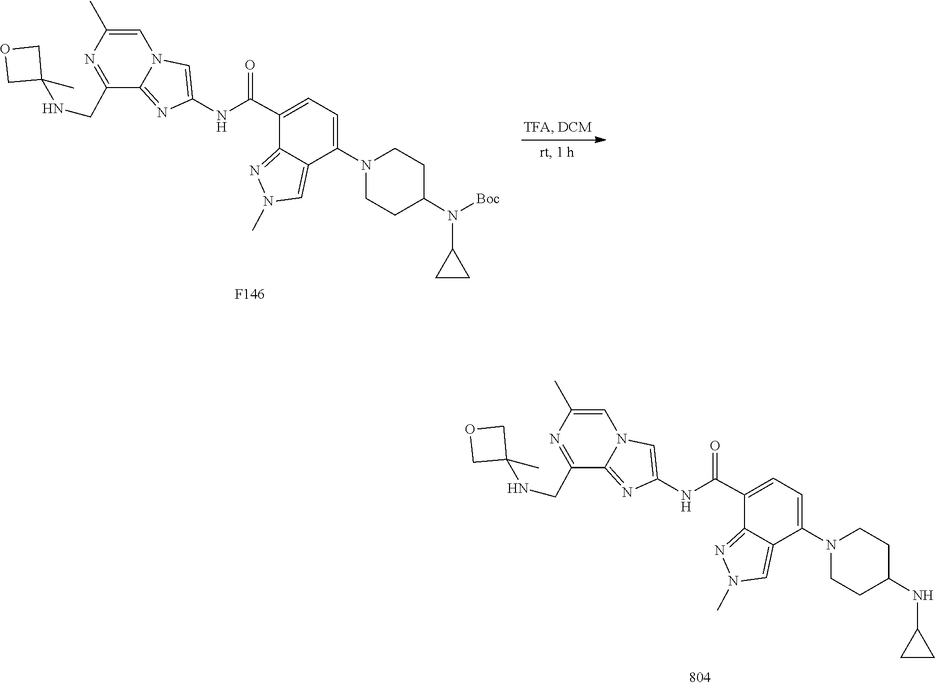 [SMILES: Cc1cn2cc(NC(=O)c3ccc(N4CCC(N(C(=O)OC(C)(C)C)C5CC5)CC4)c4cn(C)nc34)nc2c(CNC2(C)COC2)n1 ; "F146" ; Cc1cn2cc(NC(=O)c3ccc(N4CCC(NC5CC5)CC4)c4cn(C)nc34)nc2c(CNC2(C)COC2)n1]

A solution of tert-butyl N-cyclopropyl-N-[1-[2-methyl-7-[[6-methyl-8-[[(3-methyloxetan-3-yl)amino]methyl]imidazo[1,2-a]pyrazin-2-yl]carbamoyl]indazol-4-yl]-4-piperidyl]carbamate (Intermediate F146, 60 mg, 37.28 μmol, 40% purity) in DCM (1 mL) was treated with TFA (0.5 mL) at room temperature. The resulting mixture was stirred for 1 h at room temperature under nitrogen atmosphere. The resulting mixture was concentrated under reduced pressure. The crude product was purified by Prep-HPLC with the following conditions (column, C18 silica gel, XBridge, 19×150 mm; mobile phase, MeCN in water (0.05% NH₃·H₂O), 30% to 65% gradient in 14 min; detector, UV 254 nm) to afford 4-[4-(cyclopropylamino)-1-piperidyl]-2-methyl-N-[6-methyl-8-[[(3-methyloxetan-3-yl)amino]methyl]imidazo[1,2-a]pyrazin-2-yl]indazole-7-carboxamide (Compound 804, 3.5 mg, 6.44 μmol). LCMS (ES, m/z): 544 [M+H]⁺. ¹H NMR (400 MHz, DMSO-d₆) δ 8.73 (s, 1H), 8.33 (d, J=3.8 Hz, 2H), 8.00 (d, J=8.2 Hz, 1H), 6.48 (d, J=8.3 Hz, 1H), 4.46 (d, J=6.0 Hz, 2H), 4.26 (s, 3H), 4.21 (d, J=6.0 Hz, 2H), 4.16 (s, 2H), 3.88 (d, J=12.9 Hz, 2H), 3.04 (t, J=11.9 Hz, 2H), 2.80-2.75 (m, 1H), 2.39 (s, 3H), 2.08 (s, 1H), 1.98 (d, J=12.5 Hz, 2H), 1.48 (s, 3H), 1.47-1.38 (m, 2H), 0.37 (d, J=6.2 Hz, 2H), 0.22 (t, J=2.8 Hz, 3H).

### Example 328. Synthesis of Compound 813

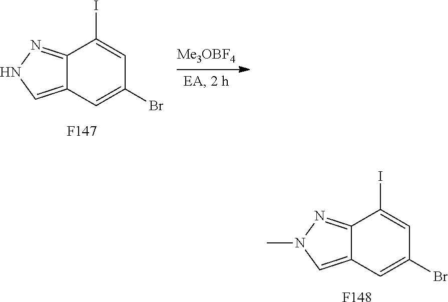

To a stirred solution of 5-bromo-7-iodo-2H-indazole (Intermediate F147, 5 g, 15.48 mmol) in EA (10 mL) was added Me₃OBF₄ (6.87 g, 46.45 mmol) at rt. The resulting mixture was stirred for 2 h. The mixture was diluted with water (10 mL) and extracted with EA (2×10 mL). The combined organic layer was washed with water (2×10 mL) and dried by anhydrous Na₂SO₄. The resulting organic layer was concentrated under reduced pressure to afford 5-bromo-7-iodo-2-methyl-indazole (Intermediate F148, 4.2 g, 12.46 mmol). LCMS (ES, m/z): 337 [M+H]⁺.

### Synthesis of Intermediate F149

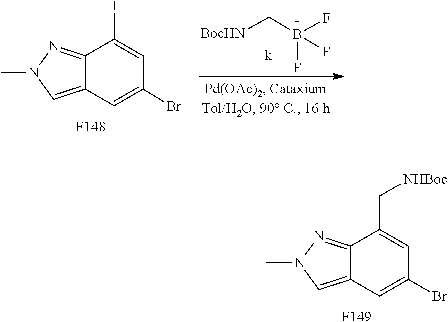

To a stirred mixture of 5-bromo-7-iodo-2-methyl-indazole (Intermediate F148, 2.8 g, 8.31 mmol) and (tert-butoxycarbonylamino)methyl-difluoro-fluoronio-boranuide (1.98 g, 9.97 mmol) in Dioxane (50 mL) were added cataCXium A (1.11 g, 1.66 mmol), Cs₂CO₃ (5.41 g, 16.62 mmol) and Pd(AcO)₂ (186.5 mg, 830.97 μmol) at room temperature under nitrogen atmosphere. The resulting mixture was stirred for 2 h at 90° C. under nitrogen atmosphere. The resulting mixture was concentrated under reduced pressure. The residue was purified by silica gel column chromatography, eluted with PE/EA (1:3) to afford tert-butyl N-[(5-bromo-2-methyl-indazol-7-yl)methyl]carbamate (Intermediate F149, 530 mg, 1.56 mmol). LCMS (ES, m/z): 340 [M+H]⁺.

### Synthesis of Intermediate F151

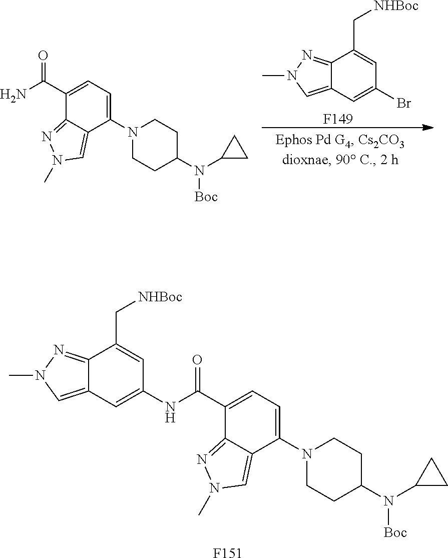

To a stirred mixture of tert-butyl N-[1-(7-carbamoyl-2-methyl-indazol-4-yl)-4-piperidyl]-N-cyclopropyl-carbamate (Intermediate F150, 50 mg, 120.92 μmol) and tert-butyl N-[(5-bromo-2-methyl-indazol-7-yl)methyl]carbamate (Intermediate F149, 49.3 mg, 145.10 μmol) in dioxane (1 mL) were added Cs₂CO₃ (78.7 mg, 241.83 μmol) and Ephos Pd G4 (11.1 mg, 12.09 μmol) at room temperature under nitrogen atmosphere. The resulting mixture was stirred for 2 h at 90° C. under nitrogen atmosphere. The resulting mixture was concentrated under reduced pressure. The residue was purified by silica gel column chromatography, eluted with PE/EA (1:3) to afford tert-butyl N-[1-[7-[[7-[(tert-butoxycarbonylamino)methyl]-2-methyl-indazol-5-yl]carbamoyl]-2-methyl-indazol-4-yl]-4-piperidyl]-N-cyclopropyl-carbamate (Intermediate F151, 45 mg, 66.88 μmol). LCMS (ES, m/z): 673 [M+H]⁺.

### Synthesis of Compound 813

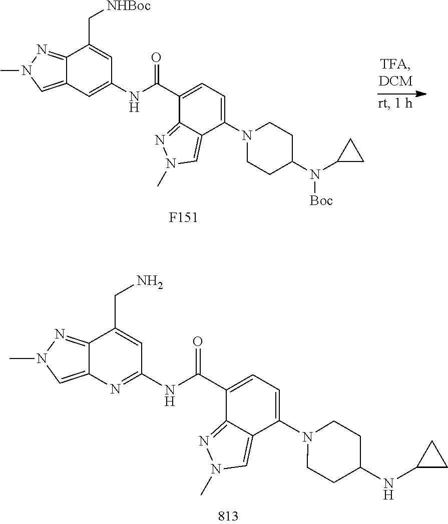

To a stirred solution of tert-butyl N-[1-[7-[[7-[(tert-butoxycarbonylamino)methyl]-2-methyl-indazol-5-yl]carbamoyl]-2-methyl-indazol-4-yl]-4-piperidyl]-N-cyclopropyl-carbamate (Intermediate F151, 45 mg, 66.88 μmol) in DCM (2 mL) was added TFA (1 mL) at rt. The resulting mixture was stirred for 1 h. The resulting mixture was concentrated under reduced pressure. The crude product was purified by Prep-HPLC with the following conditions (SunFire Prep C18 OBD Column 19*150 mm, 5 Um 10 nm, mobile phase, MeCN in water (0.05% NH₄OH), 40% to 50% gradient in 7 min; detector, UV 254 nm) to afford N-[7-(aminomethyl)-2-methyl-indazol-5-yl]-4-[4-(cyclopropylamino)-1-piperidyl]-2-methyl-indazole-7-carboxamide (Compound 813, 9 mg, 19.04 μmol). LCMS (ES, m/z): 473 [M+H]⁺. ¹H NMR (300 MHz, DMSO-d₆) δ 11.01 (s, 1H), 8.66 (s, 1H), 8.20 (d, J=6.8 Hz, 2H), 8.00 (d, J=8.1 Hz, 1H), 7.33 (s, 1H), 6.49 (d, J=8.1 Hz, 1H), 4.31 (s, 3H), 4.17 (s, 3H), 4.09 (s, 2H), 3.87 (d, J=12.3 Hz, 2H), 3.20-3.19 (m, 1H), 2.88-2.80 (m, 1H), 2.20-2.15 (m, 1H), 2.10-2.03 (m, 4H), 1.54 (d, J=11.7 Hz, 2H), 0.42 (d, J=5.9 Hz, 2H), 0.31-0.23 (m, 2H).

### Example 329. Synthesis of Compound 816

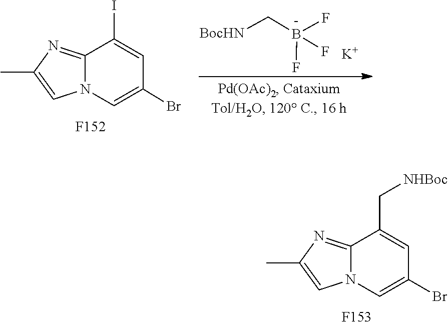

To a stirred mixture of 6-bromo-8-iodo-2-methyl-imidazo[1,2-a]pyridine (Intermediate F152, 5 g, 14.84 mmol) and potassium (((tert-butoxycarbonyl)amino)methyl)trifluoroborate (7.04 g, 29.68 mmol) in toluene (75 mL) and H₂O (7.5 mL) were added cataCXium (1.06 g, 2.97 mmol) and Pd(OAc)₂ (333.1 mg, 1.48 mmol) at room temperature under nitrogen atmosphere. The resulting mixture was stirred for 16 h at 120° C. under nitrogen atmosphere. The mixture was allowed to cool down to room temperature. The resulting mixture was diluted with water (50 mL). The resulting mixture was extracted with EA (2×60 mL). The combined organic layers were washed with brine (1×70 mL), dried over anhydrous Na₂SO₄. After filtration, the filtrate was concentrated under reduced pressure. The residue was purified by silica gel column chromatography, eluted with PE/EA (1:1) to afford tert-butyl N-[(6-bromo-2-methyl-imidazo[1,2-a]pyridin-8-yl)methyl]carbamate (Intermediate F153, 1.7 g, 5.00 mmol). LCMS (ES, m/z): 340 [M+H]⁺.

### Synthesis of Intermediate F154

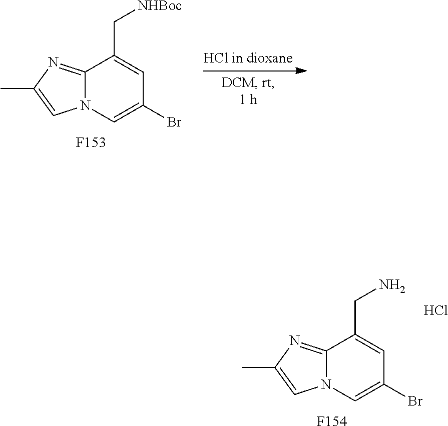

A solution of tert-butyl N-[(6-bromo-2-methyl-imidazo[1,2-a]pyridin-8-yl)methyl]carbamate (Intermediate F153, 1.7 g, 2.70 mmol) in DCM (4 mL) was treated with HCl (4.0 M in 1,4-dioxane) (2 mL) at room temperature. The resulting mixture was stirred for 1 h at room temperature under nitrogen atmosphere. The resulting mixture was concentrated under reduced pressure to afford (6-bromo-2-methyl-imidazo[1,2-a]pyridin-8-yl) methanamine hydrochloride (Intermediate F154, 1 g, 1.74 mmol). LCMS (ES, m/z): 240 [M+H]⁺.

### Synthesis of Intermediate F155

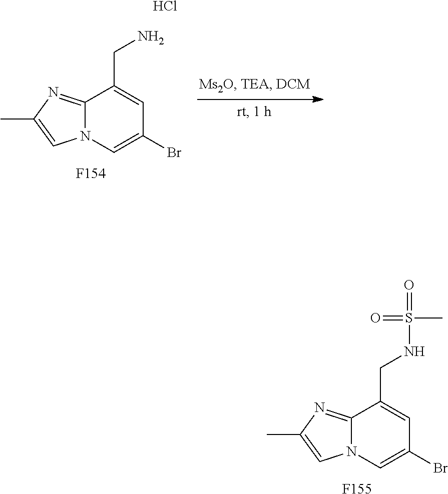

To a stirred solution of (6-bromo-2-methylimidazo[1,2-a]pyridin-8-yl) methanamine hydrochloride (Intermediate F154, 1 g, 1.74 mmol) and DIEA (672.3 mg, 5.21 mmol) in DCM (10 mL) was added Ms₂O (362.3 mg, 2.08 mmol) dropwise at 0° C. under N₂ atmosphere. The resulting mixture was stirred for 1 h at rt under N₂ atmosphere. The resulting mixture was diluted with water (6 mL). The resulting mixture was extracted with DCM (3×10 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (1:2) to afford N-[(6-bromo-2-methyl-imidazo[1,2-a]pyridin-8-yl)methyl]methanesulfonamide (Intermediate F155, 240 mg, 754.27 μmol). LCMS (ES, m/z): 318 [M+H]⁺.

### Synthesis of Intermediate F157

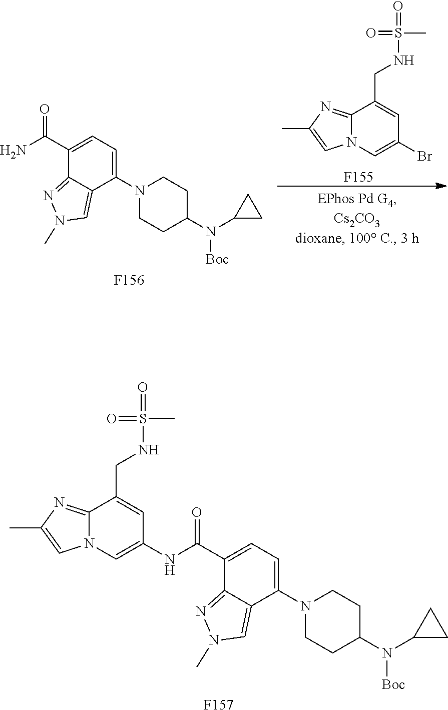

To a stirred mixture of N-[(6-bromo-2-methyl-imidazo[1,2-a]pyridin-8-yl)methyl]methanesulfonamide (Intermediate F155, 50 mg, 157.14 μmol) and tert-butyl N-[1-(7-carbamoyl-2-methyl-indazol-4-yl)-4-piperidyl]-N-cyclopropyl-carbamate (Intermediate F156, 64.9 mg, 157.14 μmol) in dioxane (2 mL) were added Cs₂CO₃ (102.4 mg, 314.28 μmol) and EPhos Pd G₄ (14.4 mg, 15.71 μmol) at room temperature under nitrogen atmosphere. The resulting mixture was stirred for 3 h at 100° C. under nitrogen atmosphere. The mixture was allowed to cool down to room temperature. The resulting mixture was concentrated off and then purified by silica gel column chromatography, eluted with PE/EA (1:2) to afford tert-butyl N-cyclopropyl-N-1-[7-[[8-(methanesulfonamidomethyl)-2-methyl-imidazo[1,2-a]pyridin-6-yl]carbamoyl]-2-methyl-indazol-4-yl]-4-piperidyl]carbamate (Intermediate F157, 33 mg, 50.71 μmol). LCMS (ES, m/z): 651 [M+H]⁺.

### Synthesis of Compound 816

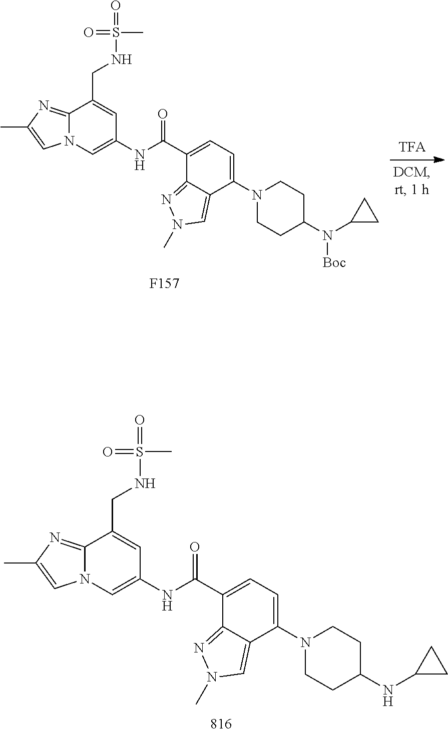 [SMILES: Cc1cn2cc(NC(=O)c3ccc(N4CCC(N(C(=O)OC(C)(C)C)C5CC5)CC4)c4cn(C)nc34)cc(CNS(C)(=O)=O)c2n1 ; Cc1cn2cc(NC(=O)c3ccc(N4CCC(NC5CC5)CC4)c4cn(C)nc34)cc(CNS(C)(=O)=O)c2n1 ; "F157"]

To a stirred mixture of tert-butyl N-cyclopropyl-N-[1-[7-[[8-(methanesulfonamidomethyl)-2-methyl-imidazo[1,2-a]pyridin-6-yl]carbamoyl]-2-methyl-indazol-4-yl]-4-piperidyl]carbamate (Intermediate F157, 33 mg, 50.71 μmol) in DCM (0.5 mL) was added TFA (0.25 mL) dropwise at room temperature. The resulting mixture was stirred for 1 h at room temperature. The resulting mixture was concentrated under reduced pressure. The crude product was purified by Prep-HPLC with the following conditions (column, C18 silica gel, XBridge, 19×150 mm; mobile phase, MeCN in water (0.05% NH₃·H₂O), 35% to 60% gradient in 14 min; detector, UV 254 nm) to afford 4-[4-(cyclopropylamino)-1-piperidyl]-N-[8-(methanesulfonamidomethyl)-2-methyl-imidazo[1,2-a]pyridin-6-yl]-2-methyl-indazole-7-carboxamide (Compound 816, 8 mg, 14.53 μmol). LCMS (ES, m/z): 551 [M+H]⁺. ¹H NMR (400 MHz, DMSO-d₆) δ 11.02 (s, 1H), 9.24 (s, 1H), 8.77 (s, 1H), 7.96 (d, J=8.0 Hz, 1H), 7.78 (s, 1H), 7.67 (t, J=6.2 Hz, 1H), 7.25 (s, 1H), 6.47 (d, J=8.2 Hz, 1H), 4.49 (d, J=6.0 Hz, 2H), 4.26 (s, 3H), 3.86 (d, J=12.8 Hz, 2H), 3.10-3.03 (m, 5H), 2.85-2.76 (m, 1H), 2.33 (s, 3H), 2.14-2.05 (m, 1H), 1.99 (d, J=12.6 Hz, 2H), 1.46 (q, J=11.3, 10.5 Hz, 2H), 0.37 (dd, J=6.7, 4.4 Hz, 2H), 0.22 (q, J=3.5, 2.8 Hz, 2H).

### Example 330. Synthesis of Compound 817

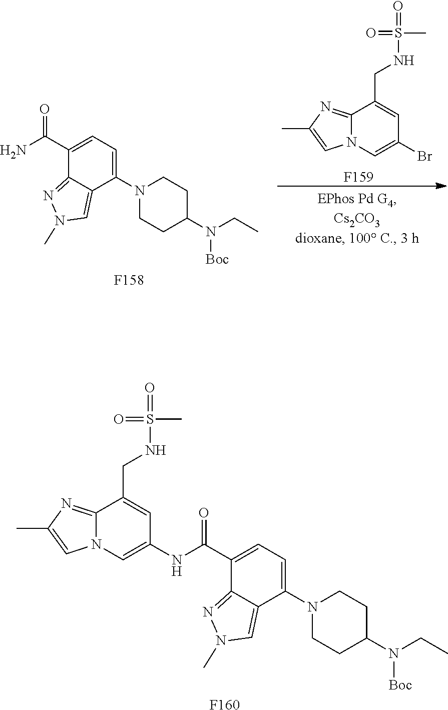

To a stirred mixture of tert-butyl N-[1-(7-carbamoyl-2-methyl-indazol-4-yl)-4-piperidyl]-N-ethyl-carbamate (Intermediate F158, 50 mg, 124.53 μmol) and N-[(6-bromo-2-methyl-imidazo[1,2-a]pyridin-8-yl)methyl]methanesulfonamide (Intermediate F159, 39.6 mg, 124.53 μmol) in dioxane (2 mL) were added Cs₂CO₃ (81.1 mg, 249.07 μmol) and EPhos Pd G₄ (11.4 mg, 12.45 μmol) at room temperature under nitrogen atmosphere. The resulting mixture was stirred for 3 h at 100° C. under nitrogen atmosphere. The mixture was allowed to cool down to room temperature. The resulting mixture was purified by silica gel column chromatography, eluted with EA to afford tert-butyl N-ethyl-N-[1-[7-[[8-(methanesulfonamidomethyl)-2-methyl-imidazo[1,2-a]pyridin-6-yl]carbamoyl]-2-methyl-indazol-4-yl]-4-piperidyl]carbamate (Intermediate F160, 35 mg, 54.79 μmol, 44.00% yield) as a light yellow solid. LCMS (ES, m/z): 639 [M+H]⁺.

### Synthesis of Compound 817

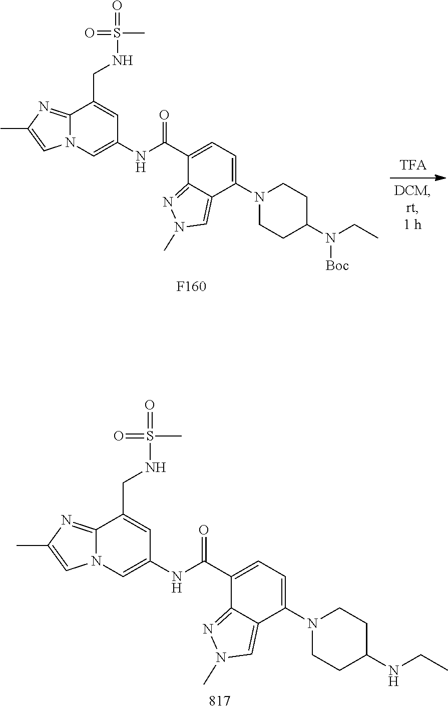

To a stirred mixture of tert-butyl N-ethyl-N-[1-[7-[[8-(methanesulfonamidomethyl)-2-methyl-imidazo[1,2-a]pyridin-6-yl]carbamoyl]-2-methyl-indazol-4-yl]-4-piperidyl]carbamate (Intermediate F160, 30 mg, 46.96 μmol) in TFA (0.25 mL) was added DCM (0.5 mL) dropwise at room temperature. The resulting mixture was stirred for 1 h at room temperature. The resulting mixture was concentrated under reduced pressure. The crude product was purified by Prep-HPLC with the following conditions (column, C18 silica gel, XBridge, 19×150 mm; mobile phase, MeCN in water (0.05% NH₃·H₂O), 30% to 65% gradient in 14 min; detector, UV 254 nm) to afford 4-[4-(ethylamino)-1-piperidyl]-N-[8-(methanesulfonamidomethyl)-2-methyl-imidazo[1,2-a]pyridin-6-yl]-2-methyl-indazole-7-carboxamide (Compound 817, 15 mg, 27.85 μmol). LCMS (ES, m/z): 539 [M+H]⁺. ¹H NMR (400 MHz, DMSO-d₆) δ 11.04 (s, 1H), 9.26 (d, J=2.0 Hz, 1H), 8.79 (s, 1H), 7.98 (d, J=8.0 Hz, 1H), 7.80 (s, 1H), 7.69 (s, 1H), 7.27 (d, J=2.0 Hz, 1H), 6.49 (d, J=8.2 Hz, 1H), 4.52 (s, 2H), 4.29 (s, 3H), 4.01-3.73 (m, 2H), 3.07 (d, J=12.1 Hz, 2H), 3.04 (s, 3H), 2.74-2.65 (m, 1H), 2.61 (q, J=7.2 Hz, 2H), 2.35 (s, 3H), 1.96 (dd, J=13.2, 4.0 Hz, 2H), 1.55-1.35 (m, 2H), 1.04 (t, J=7.1 Hz, 3H).

### Synthesis of Compound 818

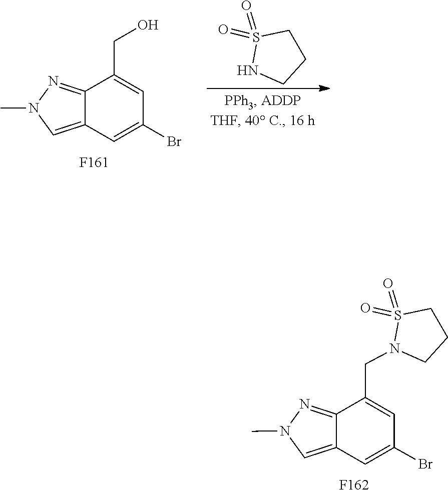

To a solution of (5-bromo-2-methyl-indazol-7-yl) methanol (Intermediate F161, 150 mg, 622.19 μmol) in THF (2 mL) were added 1,2-thiazolidine 1,1-dioxide (150.7 mg, 1.24 mmol) and PPh₃ (326.4 mg, 1.24 mmol) at rt. Then ADDP (313.9 mg, 1.24 mmol) was added at 0° C. under N₂. The mixture was stirred for 16 h at 40° C. The reaction was diluted with H₂O (15 mL). The resulting mixture was extracted with EA (2×15 mL). The combined organic layers were washed with H₂O (2×15 mL), dried over anhydrous Na₂SO₄. After filtration, the filtrate was concentrated under reduced pressure. The residue was purified by silica gel column chromatography, eluted with PE/THF (1/1) to afford 2-[(5-bromo-2-methyl-indazol-7-yl)methyl]-1,2-thiazolidine 1,1-dioxide (Intermediate F162, 90 mg, 261.46 μmol). LCMS (ES, m/z): 344 [M+H]⁺.

### Synthesis of Intermediate 164

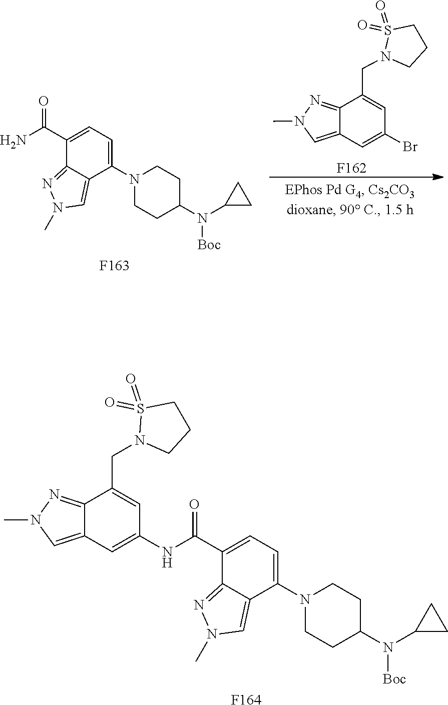

To a stirred mixture of tert-butyl N-[1-(7-carbamoyl-2-methyl-indazol-4-yl)-4-piperidyl]-N-cyclopropyl-carbamate (Intermediate F163, 80 mg, 193.46 μmol) in dioxane (1 mL) were added 2-[(5-bromo-2-methyl-indazol-7-yl)methyl]-1,2-thiazolidine 1,1-dioxide (Intermediate F162, 86.5 mg, 251.50 μmol), Cs₂CO₃ (126.0 mg, 386.93 μmol) and EPhos Pd G4 (17.7 mg, 19.35 μmol) at rt under N₂. The mixture was stirred for 1.5 h at 90° C. The reaction was diluted with H₂O (10 mL). The resulting mixture was extracted with DCM (2×10 mL). The combined organic layers were washed with H₂O (10 mL), dried over anhydrous Na₂SO₄. After filtration, the filtrate was concentrated under reduced pressure. The residue was purified by silica gel column chromatography, eluted with DCM/EA (1/3) to afford tert-butyl N-cyclopropyl-N-[1-[7-[[7-[(1,1-dioxo-1,2-thiazolidin-2-yl)methyl]-2-methyl-indazol-5-yl]carbamoyl]-2-methyl-indazol-4-yl]-4-piperidyl]carbamate (Intermediate F164, 90 mg, 132.97 μmol). LCMS (ES, m/z): 677 [M+H]⁺.

### Synthesis of Compound 818

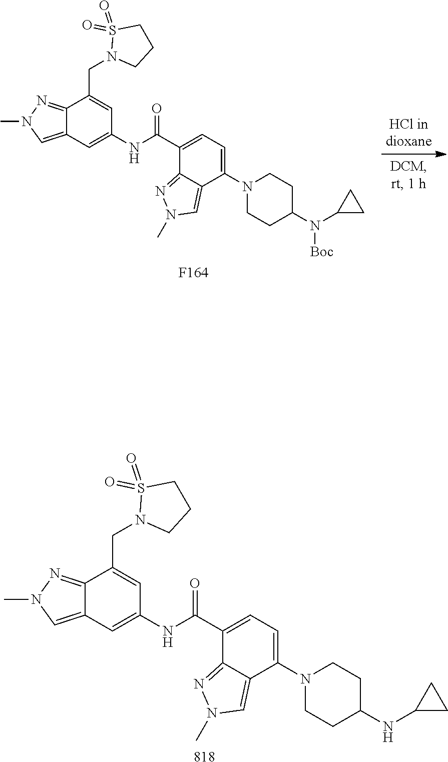

To a solution of tert-butyl N-cyclopropyl-N-[1-[7-[[7-[(1,1-dioxo-1,2-thiazolidin-2-yl)methyl]-2-methyl-indazol-5-yl]carbamoyl]-2-methyl-indazol-4-yl]-4-piperidyl]carbamate (Intermediate F164, 90 mg, 132.97 μmol) in DCM (1.5 mL) was added HCl (4.0 M in 1,4-dioxane) (330 μL). The mixture was stirred for 1 h at rt. The resulting mixture was concentrated under reduced pressure. The crude product was purified by Prep-HPLC with the following conditions (column, C18 silica gel, XBridge, 19×150 mm; mobile phase, MeCN in water (0.05% HCl), 35% to 50% gradient in 7 min; detector, UV 254 nm) to afford 4-(4-(cyclopropylamino)piperidin-1-yl)-N-(7-((1,1-dioxidoisothiazolidin-2-yl)methyl)-2-methyl-2H-indazol-5-yl)-2-methyl-2H-indazole-7-carboxamide hydrochloride (Compound 818, 35 mg, 57.08 μmol). LCMS (ES, m/z): 577 [M+H]⁺. ¹H NMR (400 MHz, DMSO-d₆) δ 11.17 (s, 1H), 9.38 (t, J=5.9 Hz, 2H), 8.83 (s, 1H), 8.32 (s, 1H), 8.25 (s, 1H), 7.98 (d, J=7.9 Hz, 1H), 7.38 (s, 1H), 6.52 (d, J=8.1 Hz, 1H), 4.46 (s, 2H), 4.28 (s, 3H), 4.16 (s, 3H), 4.00 (d, J=12.8 Hz, 2H), 3.46-3.34 (m, 1H), 3.29 (t, J=7.2 Hz, 4H), 2.99 (t, J=12.2 Hz, 2H), 2.72 (s, 1H), 2.26 (td, J=13.2, 11.7, 5.7 Hz, 4H), 1.93-1.73 (m, 2H), 0.94 (q, J=4.9, 3.9 Hz, 2H), 0.79 (t, J=6.5 Hz, 2H).

### Synthesis of Compound 820

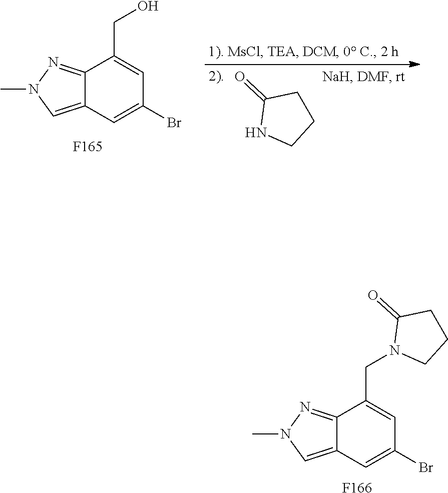

To a mixture of (5-bromo-2-methyl-indazol-7-yl) methanol (Intermediate F165, 100 mg, 414.79 μmol) in DCM (2 mL) was added TEA (125.9 mg, 1.24 mmol) and MsCl (14.1 mg, 622.19 μmol) dropwise at 0° C. The resulting mixture was stirred at 2 h at room temperature. The resulting mixture was concentrated under reduced pressure. The residue was dissolved in DMF (1 mL). The solution was added dropwise to a mixture of pyrrolidin-2-one (42.3 mg, 497.75 μmol) in DMF (2 mL) which was treated with NaH (19.9 mg, 829.59 μmol) at 0° C. for 30 minutes at rt. The mixture was stirred for 2 h at room temperature. The reaction was quenched by the addition of cold water (1×10 mL) at room temperature. The resulting mixture was extracted with EA (2×10 mL). The combined organic layers were washed with NH₄Cl (2×10 mL), dried over anhydrous Na₂SO₄. After filtration, the filtrate was concentrated under reduced pressure. The residue was purified by silica gel column chromatography, eluted with PE/EA (4:1) to afford 1-[(5-bromo-2-methyl-indazol-7-yl)methyl]pyrrolidin-2-one (Intermediate F166, 70 mg, 227.14 μmol). LCMS (ES, m/z): 318 [M+H]⁺.

### Synthesis of Intermediate F168

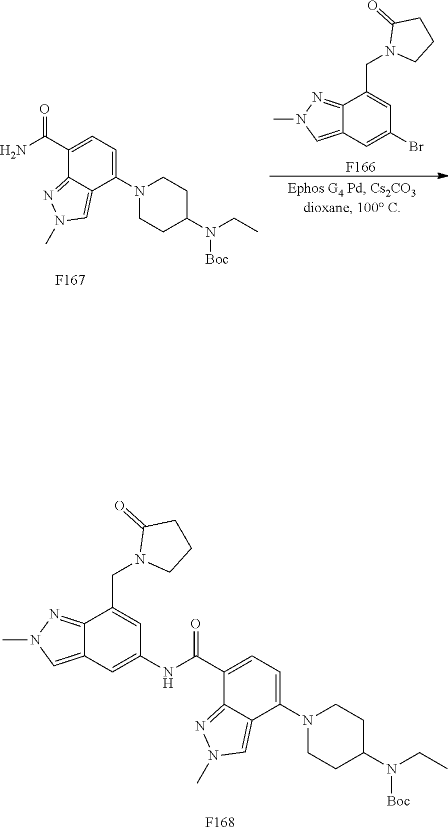

To a stirred mixture of tert-butyl N-[1-(7-carbamoyl-2-methyl-indazol-4-yl)-4-piperidyl]-N-ethyl-carbamate (Intermediate F167, 50 mg, 124.53 μmol) and 1-[(5-bromo-2-methyl-indazol-7-yl)methyl]pyrrolidin-2-one (Intermediate F166, 46.0 mg, 149.44 μmol) in Dioxane (1 mL) were added Ephos G4 Pd (11.4 mg, 12.45 μmol) and Cs₂CO₃ (81.1 mg, 249.07 μmol) at room temperature under nitrogen atmosphere. The resulting mixture was stirred for 2 h at 100° C. under nitrogen atmosphere. The resulting mixture was concentrated under reduced pressure. The residue was purified by silica gel column chromatography, eluted with PE/EA (1:3) to afford tert-butyl N-ethyl-N-[1-[2-methyl-7-[[2-methyl-7-[(2-oxopyrrolidin-1-yl)methyl]indazol-5-yl]carbamoyl]indazol-4-yl]-4-piperidyl]carbamate (Intermediate F168, 50 mg, 79.52 μmol). LCMS (ES, m/z): 629 [M+H]⁺.

### Synthesis of Compound 820

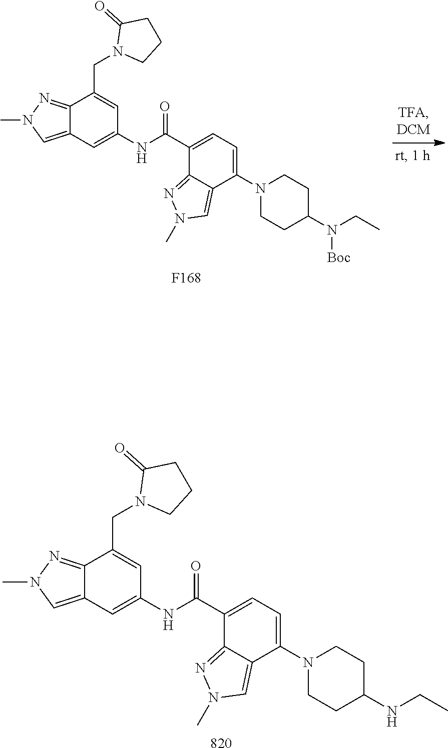

To a stirred solution of tert-butyl N-ethyl-N-[1-[2-methyl-7-[[2-methyl-7-[(2-oxopyrrolidin-1-yl)methyl]indazol-5-yl]carbamoyl]indazol-4-yl]-4-piperidyl]carbamate (Intermediate F168, 50 mg, 79.52 μmol) in DCM (2 mL) was added TFA (1 mL) at rt. The resulting mixture was stirred for 2 h. The resulting mixture was concentrated under reduced pressure. The crude product was purified by Prep-HPLC with the following conditions (SunFire Prep C18 OBD Column 19*150 mm, 5 Um 10 nm, mobile phase, MeCN in water (0.05% NH₄OH), 20% to 30% gradient in 7 min; detector, UV 254 nm) to afford 4-[4-(ethylamino)-1-piperidyl]-2-methyl-N-[2-methyl-7-[(2-oxopyrrolidin-1-yl)methyl]indazol-5-yl]indazole-7-carboxamide (Compound 820, 14 mg, 26.48 μmol). LCMS (ES, m/z): 529 [M+H]⁺. ¹H NMR (400 MHz, DMSO-d₆) δ 11.13 (s, 1H), 8.76 (s, 1H), 8.32 (s, 1H), 8.25 (d, J=1.9 Hz, 1H), 7.97 (d, J=8.0 Hz, 1H), 7.11 (s, 1H), 6.48 (d, J=8.2 Hz, 1H), 4.72 (s, 2H), 4.29 (s, 3H), 4.18 (s, 3H), 3.86 (d, J=12.7 Hz, 2H), 3.44 (t, J=7.0 Hz, 2H), 3.03 (t, J=11.7 Hz, 2H), 2.61 (q, J=7.3 Hz, 2H), 2.37 (t, J=8.1 Hz, 2H), 2.08-1.93 (m, 4H), 1.47-1.42 (d, J=10.9 Hz, 1H), 1.04 (t, J=7.0 Hz, 3H).

### Example 331: Exemplary Splicing Assay for Monitoring Expression Levels of Splice Variants

Compounds described herein were used to modulate RNA transcript abundance in cells. The expression of a target mRNA was measured by detecting the formation of an exon-exon junction in the canonical transcript (CJ). A compound mediated exon-inclusion event was detected by observing an increase in formation of a new junction with an alternative exon (AJ). Real-time qPCR assays were used to detect these splicing switches and interrogate the potency of various compounds towards different target genes. A high-throughput real time quantitative PCR (RT-qPCR) assay was developed to measure these two isoforms of the mRNA (CJ and AJ) for exemplary genes, such as HTT, SMN2, and MYB, together with a control housekeeping gene, GAPDH or GUSB or PPIA, used for normalization. Briefly, the A673 or K562 cell line was treated with various compounds described herein (e.g., compounds of Formula (I)). After treatment, the levels of the HTT, MYB, or SMN2 mRNA targets were determined from each sample of cell lysate by cDNA synthesis followed by qPCR.

### Materials:

Cells-to-C_{T} 1-step kit: ThermoFisher A25602, Cells-to-C_{T} lysis reagent: ThermoFisher 4391851C, TaqMan™ Fast Virus 1-Step Master Mix: ThermoFisher 4444436
GAPDH: VIC-PL, ThermoFisher 4326317E (Assay: Hs99999905_m1)—used for K562/suspension cell lines
GUSB: VIC-PL, ThermoFisher 4326320E (Assay: Hs99999908_m1)—used for K562/suspension cell lines
PPIA: VIC-PL, ThermoFisher 4326316E (Assay: Hs99999904_m1)—used for A673/adherent cell lines

### Probe/Primer Sequences

### Canonical Junction (CJ)

HTT Primer 1:
TCCTCCTGAGAAAGAGAAGGAC
HTT Primer 2:
GCCTGGAGATCCAGACTCA
HTT CY5-Probe:
/5Cy5/TGGCAACCCTTGAGGCCCTGTCCT/3IAbRQSp/
MYB Primer 1:
CCTCATTGGTCACAAATTGACTG
MYB Primer 2:
TGGAGAGCTTTCTAAGATTGACC
MYB CY5-Probe:
/5Cy5/AGGAAAATACTGTTTTTAGAACCCCAG/3IAbRQSp/

### Alternative Junction (AJ)

HTT Primer 1:
TCCTGAGAAAGAGAAGGACATTG
HTT Primer 2:
CTGTGGGCTCCTGTAGAAATC
HTT FAM-Probe:
/56-FAM/TGGCAACCC/ZEN/TTGAGAGGCAAGCCCT/3IABkFQ/
MYB Primer 1:
CAACACCATTTCATAGAGACCAGAC
MYB Primer 2:
GTTCTAAAATCATCCCTTGGCTTCTAAT
MYB FAM-Probe:
/56-
FAM/AAATACTGT/ZEN/ATAGGACCTCTTCTGACATCC/3IABkFQ/

### Description

The A673 cell line was cultured in DMEM with 10% FBS. Cells were diluted with full growth media and plated in a 96-well plate (15,000 cells in 100 ul media per well). The plate was incubated at 37° C. with 5% CO₂ for 24 hours to allow cells to adhere. An 11-point 3-fold serial dilution of the compounds was made in DMSO then diluted in media in an intermediate plate. Compounds were transferred from the intermediate plate to the cell plate with the top dose at a final concentration of 10 uM in the well. Final DMSO concentration was kept at or below 0.25%. The cell plate was returned to the incubator at 37° C. with 5% CO₂ for an additional 24 hours.

The K562 cell line was cultured in IMDM with 10% FBS. For K562, cells were diluted with full growth media and plated in either a 96-well plate (50,000 cells in 50 uL media per well) or a 384-well plate (8,000-40,000 cells in 45 uL media per well). An 11-point 3-fold serial dilution of the compounds were made in DMSO then diluted in media in an intermediate plate. Compound was transferred from the intermediate plate to the cell plate with the top dose at a final concentration of 10 uM in the well. Final DMSO concentration was kept at or below 0.25%. Final volume was 100 uL for 96-well plate and 50 uL for 384-well plate. The cell plate was then placed in an incubator at 37° C. with 5% CO₂ for 24 hours.

The cells were then gently washed with 50 uL-100 uL cold PBS before proceeding to addition of lysis buffer. 30 uL-50 uL of room temperature lysis buffer with DNAse I (and optionally RNAsin) was added to each well. Cells were shaken/mixed thoroughly at room temperature for 5-10 minutes for lysis to take place and then 3 uL-5 uL of room temperature stop solution was added and wells were shaken/mixed again. After 2-5 minutes, the cell lysate plate was transferred to ice for RT-qPCR reaction setup. The lysates could also be frozen at −80° C. for later use.

In some cases, a direct lysis buffer was used. An appropriate volume of 3× lysis buffer (10 mM Tris, 150 mM NaCl, 1.5%-2.5% Igepal and 0.1-1 U/uL RNAsin, pH 7.4) was directly added to either K562 or A673 cells in media and mixed by pipetting 3 times. The plates were then incubated at room temperature with shaking/rocking for 20-50 minutes to allow for lysis to take place. After this time, the cell lysate plate was transferred to ice to set up for the RT-qPCR reactions. The lysates could also be frozen at −80° C. for later use.

To set up 10 uL RT-qPCR reactions, cell lysates were transferred to 384-well qPCR plates containing the master mix according to the table below. The plates were sealed, gently vortexed, and spun down before the run. The volumes were adjusted accordingly in some instances where the reaction was carried in 20 uL. The table below summarizes the components of the RT-qPCR reactions:

| Component | 1X |
|---|---|
| Taqman 1-step RT-qPCR mix (4X) | 2.5 |
| 20X AJ Primers + Probe (FAM) | 0.5 |
| 20X CJ Primers + Probe (CY5 | 0.5 |
| 20X PPIA Control (VIC) | 0.5 |
| Cell lysate (1X) | 1-2 |
| H₂O | 4-5 |
| Total volume | 10 |

The RT-qPCR reaction was performed using a QuantStudio (ThermoFisher) under the following fast cycling conditions. All samples and standards were analyzed at least in duplicate. In some instances, bulk room temperature (RT) step of 5-10 minutes was completed for all plates before proceeding with qPCR. The table below summarizes the PCR cycle:

| Step | # cycles | Temp. | Time |
|---|---|---|---|
| RT step | 1 | 50° C. | 5 min |
| RT inactivation/initial | 1 | 95° C. | 20 sec |
| denaturation | | | |
| Amplification | 40 | 95° C. | 3 sec |
| | | 60° C. | 30 sec |

The data analysis was performed by first determining the ΔCt vs the housekeeper gene. This ΔCt was then normalized against the DMSO control (ΔΔCt) and converted to RQ (relative quantification) using the 2\hat{ }(−ΔΔCt) equation. The RQ were then converted to a percentage response by arbitrarily setting an assay window of 3.5 and 4.0 ΔCt for HTT-CJ and MYB-CJ respectively and an assay window of 9 and 3 ΔCt for HTT-AJ and MYB-AJ in 96 well format (50,000 K562 cells/well and 15,000 A673 cells per well) and an assay window of 3 and 4 ΔCt for HTT-CJ and MYB-CJ respectively and an assay window of 5 and 3 ΔCt for HTT-AJ and MYB-AJ respectively in 384 well format (8,000 K562 cells/well example). These assay windows correspond to the maximal modulation observed at high concentration of the most active compounds. The percentage response was then fitted to the 4 parametric logistic equation to evaluate the concentration dependence of compound treatment. The increase in AJ mRNA is reported as AC₅₀ (compound concentration having 50% response in AJ increase) while the decrease in CJ mRNA levels is reported as IC₅₀ (compound concentration having 50% response in CJ decrease).

A summary of these results is illustrated in Table 2, wherein “A” represents an AC₅₀/IC₅₀ of less than 100 nM; “B” represents an AC₅₀/IC₅₀ of between 100 nM and 1 μM; and “C” represents an AC₅₀/IC₅₀ of between 1 μM and 10 μM; and “D” represents an AC₅₀/IC₅₀ of greater than 10 μM.

TABLE 2
Modulation of RNA Splicing by Exemplary Compounds (K562.3)

| Cmpd | HTT | HTT | MYB | MYB |
|---|---|---|---|---|
| No. | CJ | AJ | CJ | AJ |
| 100 | C | C | B | B |
| 101 | C | C | B | B |
| 104 | C | C | A | A |
| 107 | C | D | B | B |
| 108 | C | D | B | B |
| 112 | C | D | B | A |
| 113 | D | D | B | B |
| 115 | C | C | B | B |
| 118 | C | D | C | C |
| 119 | C | D | B | B |
| 122 | C | C | B | B |
| 124 | D | D | C | B |
| 125 | D | D | B | B |
| 127 | C | D | D | D |
| 128 | B | C | B | B |
| 129 | C | C | B | B |
| 132 | C | D | B | B |
| 135 | B | C | B | B |
| 136 | C | C | B | B |
| 137 | D | D | D | D |
| 140 | C | C | B | B |
| 143 | C | C | A | A |
| 144 | D | D | A | A |
| 146 | D | D | D | D |
| 149 | C | D | C | C |
| 152 | D | D | C | C |
| 153 | B | C | A | A |
| 154 | D | D | B | B |
| 155 | D | D | C | D |
| 157 | D | D | B | A |
| 160 | D | D | D | D |
| 163 | B | B | B | B |
| 166 | D | D | B | B |
| 169 | C | C | B | B |
| 170 | C | C | A | B |
| 171 | D | D | C | D |
| 172 | D | D | D | C |
| 173 | C | C | A | A |
| 174 | C | C | B | B |
| 175 | B | C | B | B |
| 176 | C | D | B | B |
| 177 | C | D | D | D |
| 178 | D | D | D | D |
| 179 | A | A | A | A |
| 180 | A | A | A | A |
| 181 | B | B | B | B |
| 182 | C | C | A | B |
| 183 | B | B | B | B |
| 184 | C | D | B | B |
| 185 | C | C | B | B |
| 186 | B | B | B | B |
| 187 | B | B | B | B |
| 188 | D | D | D | D |
| 189 | D | D | D | D |
| 190 | D | D | D | D |
| 191 | C | C | B | B |
| 192 | B | C | B | B |
| 193 | D | D | B | C |
| 194 | C | C | B | B |
| 195 | D | D | D | D |
| 196 | B | B | B | B |
| 197 | A | A | A | A |
| 198 | D | D | D | D |
| 199 | D | D | B | B |
| 200 | B | C | B | A |
| 201 | C | C | C | C |
| 202 | C | C | A | B |
| 203 | B | C | A | A |
| 204 | B | B | A | A |
| 205 | C | C | A | A |
| 206 | B | B | A | A |
| 207 | A | A | A | A |
| 208 | B | B | A | A |
| 209 | B | B | A | A |
| 210 | B | B | A | B |
| 211 | B | B | A | A |
| 212 | D | D | C | D |
| 213 | C | D | D | D |
| 214 | C | D | D | D |
| 215 | D | D | D | D |
| 220 | C | D | B | B |
| 221 | D | D | B | B |
| 225 | C | C | C | C |
| 230 | D | D | D | D |
| 237 | C | C | C | C |
| 242 | D | D | D | D |
| 243 | D | D | D | D |
| 247 | D | D | D | D |
| 248 | D | D | C | C |
| 249 | C | C | B | B |
| 250 | D | D | D | D |
| 251 | D | D | D | D |
| 252 | B | B | B | B |
| 257 | D | D | D | D |
| 261 | D | D | D | D |
| 263 | B | C | B | B |
| 266 | D | D | B | B |
| 267 | D | D | D | D |
| 268 | B | C | A | B |
| 275 | C | C | B | B |
| 277 | C | A | B | B |
| 279 | D | D | D | D |
| 284 | C | D | C | C |
| 285 | D | D | A | A |
| 288 | D | D | A | A |
| 291 | C | C | A | A |
| 294 | B | C | A | A |
| 297 | D | D | B | B |
| 606 | D | D | D | D |
| 607 | B | C | A | A |
| 635 | C | C | B | B |

TABLE 2A
Modulation of RNA Splicing by Exemplary Compounds (THP)

| Cmpd | HTT | HTT | MYB | MYB |
|---|---|---|---|---|
| No. | CJ | AJ | CJ | AJ |
| 104 | — | — | A | A |
| 113 | D | D | B | B |
| 118 | D | D | B | C |
| 119 | D | D | B | B |
| 128 | C | C | B | B |
| 129 | C | C | A | B |
| 132 | — | — | B | B |
| 135 | — | — | B | A |
| 136 | — | — | B | B |
| 137 | — | — | D | D |
| 140 | — | — | B | B |
| 143 | — | — | A | A |
| 144 | D | D | A | A |
| 146 | — | — | D | D |
| 149 | D | D | C | D |
| 152 | D | D | C | D |
| 153 | B | B | A | A |
| 154 | D | D | C | B |
| 155 | D | D | D | C |
| 157 | D | D | A | A |
| 160 | D | D | D | D |
| 163 | B | C | C | B |
| 166 | D | D | C | B |
| 169 | — | — | B | A |
| 173 | — | — | A | A |
| 174 | — | — | B | A |
| 175 | C | C | A | A |
| 176 | D | C | B | B |
| 177 | D | D | C | D |
| 178 | D | D | D | D |
| 179 | A | A | A | A |
| 180 | A | A | A | A |
| 181 | C | C | B | B |
| 182 | C | C | A | A |
| 183 | B | B | A | A |
| 184 | D | D | B | B |
| 185 | C | C | B | A |
| 186 | B | B | B | B |
| 187 | B | B | B | B |
| 188 | D | D | D | D |
| 189 | D | B | D | D |
| 190 | D | D | D | D |
| 191 | C | C | B | A |
| 192 | C | C | B | B |
| 193 | C | C | B | B |
| 194 | C | D | B | B |
| 195 | D | D | D | D |
| 196 | B | B | B | B |
| 197 | A | A | A | A |
| 198 | D | D | D | D |
| 199 | D | D | B | B |
| 200 | B | C | A | A |
| 201 | C | C | C | C |
| 202 | C | B | A | A |
| 203 | B | B | A | A |
| 204 | B | B | A | A |
| 205 | B | B | A | A |
| 206 | B | B | A | A |
| 207 | A | A | A | A |
| 208 | A | B | A | A |
| 209 | B | B | A | A |
| 210 | A | B | A | A |
| 212 | — | — | D | D |
| 213 | — | — | C | D |
| 220 | D | C | B | B |
| 221 | C | C | B | B |
| 225 | C | D | B | C |
| 237 | C | C | C | C |
| 242 | D | D | D | D |
| 243 | D | D | D | D |
| 247 | D | D | D | D |
| 248 | D | D | D | C |
| 249 | C | D | B | B |
| 250 | D | D | C | C |
| 251 | D | D | D | D |
| 252 | B | C | B | B |
| 257 | D | D | D | D |
| 261 | D | D | D | D |
| 263 | — | — | B | B |
| 264 | D | D | D | D |
| 267 | — | — | D | D |
| 268 | — | — | A | A |
| 275 | C | C | B | B |
| 277 | C | C | B | B |
| 279 | D | D | D | D |
| 284 | D | D | D | C |
| 285 | D | D | A | A |
| 288 | D | D | A | A |
| 291 | D | C | A | A |
| 294 | C | C | A | A |
| 297 | D | D | C | B |
| 298 | C | C | B | B |
| 299 | D | D | D | D |
| 300 | D | D | D | D |
| 301 | C | D | D | D |
| 302 | D | D | D | D |
| 303 | D | D | D | D |
| 304 | D | D | D | D |
| 305 | D | D | D | D |
| 306 | D | C | A | A |
| 307 | C | C | B | B |
| 308 | D | D | D | D |
| 309 | D | D | D | D |
| 310 | D | D | D | D |
| 311 | D | D | D | D |
| 312 | C | C | C | C |
| 313 | D | D | D | C |
| 314 | C | C | B | A |
| 315 | D | D | D | C |
| 316 | D | D | C | D |
| 317 | D | D | D | D |
| 318 | D | D | D | D |
| 319 | D | D | D | D |
| 320 | B | B | B | B |
| 321 | B | A | A | A |
| 322 | C | C | C | C |
| 323 | C | C | B | B |
| 324 | D | D | C | B |
| 325 | C | C | B | A |
| 326 | C | C | B | B |
| 327 | C | C | B | A |
| 328 | B | B | A | A |
| 329 | D | C | B | B |
| 330 | D | D | C | B |
| 331 | B | B | A | A |
| 332 | D | D | B | B |
| 333 | D | D | D | D |
| 334 | D | C | B | A |
| 335 | A | A | A | A |
| 336 | D | D | D | D |
| 337 | D | D | C | C |
| 338 | C | C | A | A |
| 339 | B | B | B | B |
| 340 | D | D | D | D |
| 341 | C | D | D | C |
| 342 | C | C | A | A |
| 343 | A | A | A | A |
| 344 | D | D | B | B |
| 345 | D | D | C | D |
| 346 | D | D | D | D |
| 347 | C | C | A | A |
| 348 | D | D | C | B |
| 349 | D | D | A | A |
| 350 | D | D | B | B |
| 351 | D | D | C | D |
| 352 | C | D | A | A |
| 353 | C | C | B | B |
| 354 | B | C | B | A |
| 355 | B | B | A | A |
| 356 | C | C | B | A |
| 357 | B | B | A | A |
| 358 | C | C | C | B |
| 359 | B | B | B | B |
| 360 | C | C | C | C |
| 361 | D | C | B | B |
| 362 | D | D | B | A |
| 363 | D | D | D | D |
| 364 | C | B | B | A |
| 365 | D | D | C | C |
| 366 | D | D | D | D |
| 367 | C | C | A | A |
| 368 | C | C | A | A |
| 369 | C | B | A | A |
| 370 | C | C | B | A |
| 371 | C | C | B | A |
| 375 | C | C | A | A |
| 376 | C | D | B | B |
| 377 | A | A | A | A |
| 378 | C | C | A | A |
| 379 | D | D | C | C |
| 380 | D | D | C | C |
| 381 | C | C | C | C |
| 382 | A | A | A | A |
| 383 | D | B | A | A |
| 384 | D | D | D | D |
| 385 | D | C | D | B |
| 386 | D | C | B | B |
| 387 | B | B | A | A |
| 388 | C | C | B | B |
| 389 | B | B | A | A |
| 390 | D | D | B | A |
| 391 | D | D | A | A |
| 392 | D | D | C | C |
| 393 | D | D | B | B |
| 394 | C | C | B | A |
| 395 | D | D | B | A |
| 396 | B | B | A | A |
| 397 | D | D | C | C |
| 398 | C | C | A | A |
| 399 | C | C | B | A |
| 400 | D | D | C | C |
| 401 | D | D | C | C |
| 402 | D | D | D | D |
| 403 | D | D | D | D |
| 404 | D | C | A | A |
| 405 | B | B | A | A |
| 406 | C | C | B | B |
| 407 | D | D | A | A |
| 408 | C | B | A | A |
| 409 | C | D | B | C |
| 410 | D | D | D | D |
| 411 | C | D | D | D |
| 412 | D | D | C | C |
| 413 | C | D | D | C |
| 414 | D | D | D | D |
| 415 | B | B | A | A |
| 416 | D | D | D | D |
| 417 | D | C | A | A |
| 418 | D | D | C | B |
| 419 | D | D | C | C |
| 420 | D | D | D | D |
| 421 | D | D | D | D |
| 422 | B | B | A | A |
| 423 | C | C | B | B |
| 424 | D | D | D | D |
| 425 | C | C | B | B |
| 426 | C | C | B | B |
| 427 | D | D | D | C |
| 428 | D | D | C | C |
| 429 | C | D | C | C |
| 431 | B | B | A | A |
| 432 | B | B | A | A |
| 433 | B | B | A | A |
| 434 | B | B | A | A |
| 435 | D | D | D | D |
| 436 | D | D | B | B |
| 437 | D | D | D | D |
| 438 | D | D | D | D |
| 439 | D | D | D | D |
| 440 | C | C | B | A |
| 441 | C | C | C | C |
| 442 | D | D | D | D |
| 443 | B | B | B | A |
| 444 | C | C | B | A |
| 445 | D | D | D | D |
| 446 | C | D | C | D |
| 447 | D | D | C | D |
| 449 | D | D | D | D |
| 450 | D | D | D | D |
| 451 | D | D | C | C |
| 452 | D | D | D | D |
| 453 | D | D | B | A |
| 454 | D | D | D | C |
| 455 | D | D | C | C |
| 456 | D | D | B | A |
| 457 | C | C | C | C |
| 458 | C | C | C | C |
| 459 | C | D | B | B |
| 460 | C | C | B | B |
| 461 | B | B | B | B |
| 462 | D | D | D | D |
| 463 | C | C | A | A |
| 464 | B | B | B | B |
| 465 | D | D | D | D |
| 466 | D | D | D | D |
| 467 | B | B | A | A |
| 468 | D | D | A | A |
| 469 | C | C | C | C |
| 470 | B | B | B | B |
| 471 | D | D | B | B |
| 472 | D | D | D | C |
| 473 | D | D | D | D |
| 474 | D | D | D | D |
| 475 | D | D | B | B |
| 476 | D | D | C | C |
| 477 | B | B | B | B |
| 478 | D | D | D | D |
| 479 | D | D | B | B |
| 481 | D | D | D | C |
| 482 | D | D | D | D |
| 483 | D | D | D | D |
| 484 | D | D | D | D |
| 485 | B | B | B | B |
| 486 | D | D | D | D |
| 487 | C | D | D | D |
| 488 | D | D | D | D |
| 489 | D | D | D | D |
| 490 | D | D | C | D |
| 491 | D | D | D | D |
| 492 | D | D | D | D |
| 493 | C | C | B | B |
| 494 | D | D | D | D |
| 495 | B | B | B | B |
| 496 | D | D | C | B |
| 497 | D | D | D | D |
| 498 | D | D | D | D |
| 499 | C | C | C | C |
| 500 | D | D | D | D |
| 501 | B | B | B | B |
| 502 | B | B | B | B |
| 505 | B | B | B | B |
| 506 | C | C | B | B |
| 507 | C | C | C | B |
| 508 | D | D | D | C |
| 509 | D | D | D | D |
| 510 | C | C | C | C |
| 511 | D | D | D | D |
| 512 | D | D | C | B |
| 513 | D | D | C | C |
| 514 | C | C | B | B |
| 515 | D | C | B | B |
| 518 | C | C | B | A |
| 519 | B | B | B | B |
| 520 | D | D | D | D |
| 521 | D | D | D | D |
| 522 | C | C | C | C |
| 523 | C | C | C | C |
| 524 | C | C | A | A |
| 525 | C | D | D | C |
| 526 | C | C | C | C |
| 527 | B | B | B | B |
| 528 | B | B | B | B |
| 529 | D | D | B | B |
| 530 | D | D | C | D |
| 531 | D | D | C | C |
| 532 | B | B | B | B |
| 533 | B | B | B | B |
| 534 | D | D | D | D |
| 535 | C | C | C | C |
| 536 | D | D | D | D |
| 537 | D | D | D | D |
| 538 | D | D | B | B |
| 539 | D | C | D | D |
| 540 | C | C | A | A |
| 541 | B | B | A | A |
| 542 | D | D | C | B |
| 543 | C | C | B | B |
| 544 | B | B | B | B |
| 545 | D | D | C | C |
| 546 | D | D | B | B |
| 547 | C | C | C | C |
| 548 | D | D | B | B |
| 549 | D | D | C | C |
| 553 | C | C | B | A |
| 557 | D | D | B | B |
| 558 | D | D | C | C |
| 559 | C | C | C | C |
| 560 | D | D | C | D |
| 561 | D | D | C | D |
| 562 | D | D | D | D |
| 563 | D | D | B | B |
| 564 | B | B | B | B |
| 565 | B | B | B | B |
| 566 | B | C | B | B |
| 567 | B | C | B | B |
| 568 | B | C | B | B |
| 569 | D | D | D | D |
| 570 | D | D | D | D |
| 571 | D | D | C | C |
| 572 | C | D | C | C |
| 573 | D | D | D | D |
| 574 | D | D | D | D |
| 575 | D | D | C | B |
| 576 | D | D | D | D |
| 577 | C | C | B | A |
| 578 | B | B | B | B |
| 579 | D | D | D | D |
| 580 | D | D | C | C |
| 581 | D | D | C | C |
| 582 | D | D | D | D |
| 583 | C | C | D | D |
| 584 | C | C | A | A |
| 588 | C | C | A | A |
| 592 | C | C | C | C |
| 593 | D | D | D | D |
| 594 | D | D | D | D |
| 595 | C | D | D | D |
| 596 | C | C | C | C |
| 597 | D | D | D | D |
| 598 | D | D | C | C |
| 599 | C | C | C | C |
| 600 | D | D | D | D |
| 601 | D | D | D | C |
| 602 | D | D | D | D |
| 603 | C | C | B | B |
| 604 | C | D | D | C |
| 605 | D | C | B | B |
| 606 | D | D | C | C |
| 607 | B | C | A | A |
| 608 | B | A | A | A |
| 609 | D | D | B | A |
| 610 | D | D | D | D |
| 611 | D | D | D | D |
| 612 | D | D | B | B |
| 613 | C | D | C | C |
| 614 | C | D | C | C |
| 615 | C | C | B | C |
| 616 | C | C | C | C |
| 617 | D | D | C | B |
| 618 | C | C | B | A |
| 619 | C | C | B | B |
| 620 | B | B | A | B |
| 621 | C | B | B | A |
| 622 | B | B | A | B |
| 623 | B | B | A | A |
| 624 | D | D | D | D |
| 625 | B | B | A | A |
| 626 | D | D | D | D |
| 627 | D | D | D | D |
| 628 | B | B | A | A |
| 629 | B | C | A | A |
| 630 | C | C | C | C |
| 631 | C | C | C | C |
| 632 | B | B | A | A |
| 633 | D | D | B | B |
| 634 | A | A | A | A |
| 635 | C | C | B | B |
| 640 | B | B | A | A |
| 641 | B | B | B | B |
| 642 | C | C | C | C |
| 643 | C | C | B | B |
| 648 | D | D | C | C |
| 649 | D | D | C | B |
| 650 | A | B | A | A |
| 651 | D | D | D | D |
| 656 | D | D | D | D |
| 661 | C | C | C | C |
| 662 | D | D | D | D |
| 663 | C | C | C | C |
| 664 | C | C | C | C |
| 665 | B | B | B | B |
| 666 | C | C | B | B |
| 667 | D | D | D | C |
| 668 | C | C | B | B |
| 669 | C | C | C | B |
| 670 | C | D | D | D |
| 671 | D | C | B | B |
| 675 | A | A | A | A |
| 679 | C | C | B | A |
| 680 | D | D | C | C |
| 681 | B | B | B | A |
| 682 | B | B | A | A |
| 686 | D | D | D | D |
| 687 | D | D | C | C |
| 688 | A | B | A | A |
| 689 | A | B | A | A |
| 690 | A | B | A | A |
| 691 | D | D | B | B |
| 692 | C | B | A | A |
| 694 | D | D | D | D |
| 695 | C | C | C | C |
| 696 | D | D | D | D |
| 697 | B | A | A | A |
| 698 | C | C | A | A |
| 699 | B | B | A | A |
| 700 | B | B | A | A |
| 701 | D | D | B | B |
| 702 | D | D | C | C |
| 703 | D | D | D | D |
| 704 | B | B | A | A |
| 705 | B | B | A | A |
| 706 | B | B | B | A |
| 707 | C | B | A | A |
| 708 | D | D | D | C |
| 709 | C | C | B | B |
| 710 | C | C | C | C |
| 711 | D | D | C | C |
| 712 | A | A | A | A |
| 713 | D | D | B | B |
| 714 | B | B | A | A |
| 715 | D | D | D | D |
| 716 | C | D | C | C |
| 717 | C | D | D | D |
| 718 | C | C | A | A |
| 719 | C | C | A | A |
| 720 | B | B | A | A |
| 721 | A | B | A | A |
| 722 | D | D | D | D |
| 723 | B | B | A | A |
| 724 | B | C | A | A |
| 725 | D | D | D | D |
| 726 | D | D | C | B |
| 727 | B | B | A | A |
| 728 | D | D | B | B |
| 729 | C | D | D | D |
| 730 | D | D | B | B |
| 731 | D | D | B | B |
| 732 | D | D | A | A |
| 733 | D | D | D | D |
| 734 | D | D | D | D |
| 735 | D | D | C | C |
| 736 | D | D | D | C |
| 737 | D | D | D | D |
| 738 | D | D | A | A |
| 739 | C | B | A | A |
| 740 | C | D | D | D |
| 741 | D | C | A | A |
| 742 | D | D | B | B |
| 743 | A | A | A | A |
| 744 | C | B | A | A |
| 745 | B | B | A | A |
| 746 | B | B | B | A |
| 747 | B | B | A | A |
| 748 | C | B | A | A |
| 749 | B | B | A | B |
| 750 | A | B | A | A |
| 751 | D | D | D | D |
| 752 | D | D | D | D |
| 753 | D | D | D | D |
| 754 | D | D | D | D |
| 755 | C | C | B | A |
| 756 | B | B | A | A |
| 757 | D | C | A | A |
| 758 | D | D | A | A |
| 759 | C | C | A | A |
| 760 | D | D | D | D |
| 761 | D | D | B | B |
| 762 | D | D | A | A |
| 763 | C | B | A | A |
| 764 | C | D | A | A |
| 765 | B | B | A | A |
| 766 | A | A | A | A |
| 767 | C | C | A | A |
| 768 | A | A | A | A |
| 769 | B | B | A | A |
| 770 | B | B | B | B |
| 771 | C | D | B | B |
| 772 | D | D | C | B |
| 773 | C | C | C | C |
| 774 | D | D | D | D |
| 775 | D | D | D | D |
| 776 | D | D | D | D |
| 777 | C | C | C | C |
| 778 | D | D | D | D |
| 779 | D | D | B | B |
| 780 | B | B | A | A |
| 781 | D | D | D | D |
| 782 | D | D | B | B |
| 783 | D | C | A | A |
| 784 | B | B | A | A |
| 785 | B | B | A | A |
| 786 | C | C | B | B |
| 787 | D | D | C | D |
| 788 | D | D | B | A |
| 789 | B | B | A | A |
| 790 | C | B | A | A |
| 791 | D | D | D | D |
| 792 | C | C | C | C |
| 793 | D | D | C | C |
| 794 | D | D | B | C |
| 795 | D | D | D | D |
| 796 | D | D | D | D |
| 797 | D | D | D | D |
| 798 | C | C | C | C |
| 799 | D | D | C | C |
| 800 | B | B | A | A |
| 801 | C | C | A | A |
| 802 | C | C | B | A |
| 803 | D | D | D | D |
| 804 | D | D | D | D |
| 805 | D | D | C | D |
| 806 | D | D | C | C |
| 807 | D | D | D | D |
| 808 | D | C | D | D |
| 809 | D | D | B | A |
| 810 | C | B | A | A |
| 811 | D | D | D | C |
| 812 | D | D | D | D |
| 813 | D | D | C | C |
| 814 | D | D | B | C |
| 815 | D | D | D | D |
| 816 | D | D | C | C |
| 817 | D | D | C | C |
| 818 | D | D | D | D |
| 819 | C | C | C | C |
| 820 | D | D | C | D |
| 821 | C | D | B | B |
| 822 | D | D | B | B |
| 823 | D | D | C | B |
| 824 | D | D | C | C |
| 825 | D | D | C | C |
| 826 | D | D | D | D |
| 827 | C | C | B | A |
| 828 | C | C | B | A |
| 829 | D | D | D | D |
| 830 | D | D | D | D |
| 831 | B | B | B | B |
| 832 | C | C | C | C |
| 833 | C | C | B | B |
| 834 | D | D | D | D |
| 835 | D | D | D | D |
| 836 | D | D | B | B |
| 837 | C | C | A | A |
| 838 | D | D | C | C |
| 839 | C | C | B | B |
| 840 | C | D | C | C |
| 841 | D | D | D | D |
| 842 | D | D | D | D |
| 843 | D | D | D | D |
| 844 | D | D | D | D |
| 845 | D | D | D | D |
| 846 | B | B | B | B |
| 847 | B | B | A | A |
| 848 | B | B | B | B |
| 849 | B | B | B | B |
| 850 | D | D | D | D |
| 851 | D | D | D | D |
| 852 | D | C | B | B |
| 853 | D | D | D | D |
| 854 | D | C | B | B |
| 855 | C | C | C | C |
| 856 | C | C | C | C |
| 857 | D | D | D | D |
| 858 | C | C | B | B |
| 859 | C | C | B | B |
| 860 | D | D | D | D |
| 861 | C | D | B | B |
| 862 | B | B | B | B |
| 863 | D | D | D | D |
| 864 | D | D | C | C |
| 865 | D | D | C | C |
| 866 | D | D | D | D |
| 867 | D | D | D | D |
| 868 | D | D | D | D |
| 869 | D | D | D | D |
| 870 | D | D | D | D |
| 871 | D | D | C | C |
| 872 | D | D | D | D |
| 873 | D | D | D | D |
| 874 | D | D | D | D |
| 875 | D | D | D | D |
| 876 | C | C | C | C |
| 877 | D | D | D | D |
| 878 | D | D | D | D |
| 879 | D | D | C | C |
| 880 | D | D | C | C |
| 881 | C | D | C | B |
| 882 | D | D | B | B |
| 883 | D | D | C | C |
| 884 | D | D | C | C |
| 885 | C | C | C | C |

Additional studies were carried out for a larger panel of genes using the protocol provided above. The junction between flanking upstream and downstream exons was used to design canonical junction qPCR assays. At least one of the forward primer, reverse primer or the CY5-labeled 5′ nuclease probe (with 3′ quencher such as ZEN/Iowa Black FQ) was designed to overlap with the exon junction to capture the CJ mRNA transcript. BLAST was used to confirm the specificity of the probeset and parameters such as melting temperature, GC content, amplicon size, and primer dimer formation are considered during their design. Data for the decrease in CJ mRNA levels for four exemplary genes (HTT, SMN2, and MYB) analyzed in this panel are reported as IC₅₀ (compound concentration having 50% response in CJ decrease).

A summary of the results from the panel is illustrated in Table 3, wherein “A” represents an IC₅₀ of less than 100 nM; “B” represents an IC₅₀ of between 100 nM and 1 μM; and “C” represents an IC₅₀ of between 1 μM and 10 μM; and “D” represents an IC₅₀ of greater than 10 μM.

TABLE 3
Modulation of RNA Splicing by Exemplary Compounds

| Cmpd | HTT | MYB | SMN2 |
|---|---|---|---|
| No. | CJ | CJ | CJ |
| 100 | C | B | A |
| 101 | C | B | A |
| 104 | C | A | A |
| 107 | C | B | A |
| 108 | C | B | A |
| 112 | C | B | A |
| 113 | D | B | A |
| 115 | C | B | A |
| 118 | C | C | B |
| 119 | C | B | B |
| 122 | C | B | A |
| 124 | D | C | A |
| 125 | D | B | A |
| 127 | C | D | B |
| 128 | B | B | A |
| 129 | C | B | A |
| 132 | C | B | A |
| 135 | B | B | A |
| 136 | C | B | A |
| 137 | D | D | B |
| 140 | C | B | A |
| 143 | C | A | A |
| 144 | D | A | A |
| 146 | D | D | C |
| 149 | C | C | B |
| 152 | D | C | B |
| 153 | B | A | A |
| 154 | D | B | A |
| 155 | D | C | C |
| 157 | D | B | A |
| 160 | D | D | D |
| 163 | B | B | A |
| 166 | D | B | A |
| 169 | C | B | A |
| 170 | C | A | A |
| 171 | D | C | C |
| 172 | D | D | C |
| 173 | C | A | A |
| 174 | C | B | A |
| 175 | B | B | A |
| 176 | C | B | A |
| 177 | C | D | D |
| 178 | D | D | C |
| 179 | A | A | A |
| 180 | A | A | A |
| 181 | B | B | A |
| 182 | C | A | A |
| 183 | B | B | A |
| 184 | C | B | A |
| 185 | C | B | A |
| 186 | B | B | B |
| 187 | B | B | B |
| 188 | D | D | D |
| 189 | D | D | D |
| 190 | D | D | D |
| 191 | C | B | A |
| 192 | B | B | A |
| 193 | D | B | A |
| 194 | C | B | A |
| 195 | D | D | C |
| 196 | B | B | A |
| 197 | A | A | A |
| 198 | D | D | D |
| 199 | D | B | A |
| 200 | B | B | A |
| 201 | C | C | B |
| 202 | C | A | A |
| 203 | B | A | A |
| 204 | B | A | A |
| 205 | C | A | A |
| 206 | B | A | A |
| 207 | A | A | A |
| 208 | B | A | A |
| 209 | B | A | A |
| 210 | B | A | A |
| 211 | B | A | A |
| 212 | D | C | C |
| 213 | C | D | D |
| 214 | C | D | D |
| 215 | D | D | C |
| 220 | C | B | B |
| 221 | D | B | A |
| 225 | C | C | B |
| 230 | D | D | C |
| 237 | C | C | C |
| 242 | D | D | C |
| 243 | D | D | C |
| 247 | D | D | C |
| 248 | D | C | B |
| 249 | C | B | A |
| 250 | D | D | C |
| 251 | D | D | C |
| 252 | B | B | A |
| 257 | D | D | C |
| 261 | D | D | D |
| 263 | B | B | A |
| 266 | D | B | A |
| 267 | D | D | D |
| 268 | B | A | A |
| 275 | C | B | A |
| 277 | C | B | A |
| 279 | D | D | D |
| 284 | C | C | B |
| 285 | D | A | A |
| 288 | D | A | A |
| 291 | C | A | A |
| 294 | B | A | A |
| 297 | D | B | A |
| 298 | C | B | A |
| 299 | D | D | D |
| 300 | D | D | C |
| 301 | C | D | C |
| 302 | D | D | D |
| 303 | D | D | D |
| 304 | D | D | B |
| 305 | D | D | D |
| 306 | D | A | A |
| 307 | C | B | A |
| 308 | D | D | B |
| 309 | D | D | D |
| 310 | D | D | — |
| 311 | D | D | — |
| 312 | C | C | — |
| 313 | D | D | — |
| 314 | C | B | — |
| 315 | D | D | — |
| 316 | D | C | — |
| 317 | D | D | — |
| 318 | D | D | — |
| 319 | D | D | — |
| 320 | B | B | — |
| 321 | B | A | — |
| 322 | C | C | — |
| 323 | C | B | — |
| 324 | D | C | — |
| 325 | C | B | — |
| 326 | C | B | — |
| 327 | C | B | — |
| 328 | B | A | — |
| 329 | D | B | — |
| 330 | D | C | — |
| 331 | B | A | — |
| 332 | D | B | — |
| 333 | D | D | — |
| 334 | D | B | — |
| 335 | A | A | — |
| 336 | D | D | — |
| 337 | D | C | — |
| 338 | C | A | — |
| 339 | B | B | — |
| 340 | D | D | — |
| 341 | C | D | — |
| 342 | C | A | — |
| 343 | A | A | — |
| 344 | D | B | — |
| 345 | D | C | — |
| 346 | D | D | — |
| 347 | C | A | — |
| 348 | D | C | — |
| 349 | D | A | — |
| 350 | D | B | — |
| 351 | D | C | — |
| 352 | C | A | — |
| 353 | C | B | — |
| 354 | B | B | — |
| 355 | B | A | — |
| 356 | C | B | — |
| 357 | B | A | — |
| 358 | C | C | — |
| 359 | B | B | — |
| 360 | C | C | — |
| 361 | D | B | — |
| 362 | D | B | — |
| 363 | D | D | — |
| 364 | C | A | — |
| 365 | D | C | — |
| 366 | D | D | — |
| 367 | C | A | — |
| 368 | C | A | — |
| 369 | C | A | — |
| 370 | C | B | — |
| 371 | C | B | — |
| 375 | C | A | — |
| 376 | C | B | — |
| 377 | A | A | — |
| 378 | C | A | — |
| 379 | D | C | — |
| 380 | D | C | — |
| 381 | C | C | — |
| 382 | A | A | — |
| 383 | D | A | — |
| 384 | D | D | — |
| 385 | D | D | — |
| 386 | D | B | — |
| 387 | B | A | — |
| 388 | C | B | — |
| 389 | B | A | — |
| 390 | D | B | — |
| 391 | D | A | — |
| 392 | D | C | — |
| 393 | D | B | — |
| 394 | C | B | — |
| 395 | D | B | — |
| 396 | B | A | — |
| 397 | D | C | — |
| 398 | C | A | — |
| 399 | C | B | — |
| 400 | D | C | — |
| 401 | D | C | — |
| 402 | D | D | — |
| 403 | D | D | — |
| 404 | D | A | — |
| 405 | B | A | — |
| 406 | C | B | — |
| 407 | D | A | — |
| 408 | C | A | — |
| 409 | C | B | — |
| 410 | D | D | — |
| 411 | C | D | — |
| 412 | D | C | — |
| 413 | C | D | — |
| 414 | D | D | — |
| 415 | B | A | — |
| 416 | D | D | — |
| 417 | D | A | — |
| 418 | D | C | — |
| 419 | D | C | — |
| 420 | D | D | — |
| 421 | D | D | — |
| 422 | B | A | — |
| 423 | C | B | — |
| 424 | D | D | — |
| 425 | C | B | — |
| 426 | C | B | — |
| 427 | D | D | — |
| 428 | D | C | — |
| 429 | C | C | — |
| 431 | B | A | — |
| 432 | B | A | — |
| 433 | B | A | — |
| 434 | B | A | — |
| 435 | D | D | — |
| 436 | D | B | — |
| 437 | D | D | — |
| 438 | D | D | — |
| 439 | D | D | — |
| 440 | C | B | — |
| 441 | C | C | — |
| 442 | D | D | — |
| 443 | B | B | — |
| 444 | C | B | — |
| 445 | D | D | — |
| 446 | C | C | — |
| 447 | D | C | — |
| 449 | D | D | — |
| 450 | D | D | — |
| 451 | D | C | — |
| 452 | D | D | — |
| 453 | D | B | — |
| 454 | D | D | — |
| 455 | D | C | — |
| 456 | D | B | — |
| 457 | C | C | — |
| 458 | C | C | — |
| 459 | C | B | — |
| 460 | C | B | — |
| 461 | B | B | — |
| 462 | D | D | — |
| 463 | C | A | — |
| 464 | B | B | — |
| 465 | D | D | — |
| 466 | D | D | — |
| 467 | B | A | — |
| 468 | D | A | — |
| 469 | C | C | — |
| 470 | B | B | — |
| 471 | D | B | — |
| 472 | D | D | — |
| 473 | D | D | — |
| 474 | D | D | — |
| 475 | D | B | — |
| 476 | D | C | — |
| 477 | B | B | — |
| 478 | D | D | — |
| 479 | D | B | — |
| 481 | D | D | — |
| 482 | D | D | — |
| 483 | D | D | — |
| 484 | D | D | — |
| 485 | B | B | — |
| 486 | D | D | — |
| 487 | C | D | — |
| 488 | D | D | — |
| 489 | D | D | — |
| 490 | D | C | — |
| 491 | D | D | — |
| 492 | D | D | — |
| 493 | C | B | — |
| 494 | D | D | — |
| 495 | B | B | — |
| 496 | D | C | — |
| 497 | D | D | — |
| 498 | D | D | — |
| 499 | C | C | — |
| 500 | D | D | — |
| 501 | B | B | — |
| 502 | B | B | — |
| 505 | B | B | — |
| 506 | C | B | — |
| 507 | C | C | — |
| 508 | D | D | — |
| 509 | D | D | — |
| 510 | C | C | — |
| 511 | D | D | — |
| 512 | D | C | — |
| 513 | D | C | — |
| 514 | C | B | — |
| 515 | D | B | — |
| 518 | C | B | — |
| 519 | B | B | — |
| 520 | D | D | — |
| 521 | D | D | — |
| 522 | C | C | — |
| 523 | C | C | — |
| 524 | C | A | — |
| 525 | C | D | — |
| 526 | C | C | — |
| 527 | B | B | — |
| 528 | B | B | — |
| 529 | D | B | — |
| 530 | D | C | — |
| 531 | D | C | — |
| 532 | B | B | — |
| 533 | B | B | — |
| 534 | D | D | — |
| 535 | C | C | — |
| 536 | D | D | — |
| 537 | D | D | — |
| 538 | D | B | — |
| 539 | D | D | — |
| 540 | C | A | — |
| 541 | B | A | — |
| 542 | D | C | — |
| 543 | C | B | — |
| 544 | B | B | — |
| 545 | D | C | — |
| 546 | D | B | — |
| 547 | C | C | — |
| 548 | D | B | — |
| 549 | D | C | — |
| 553 | C | B | — |
| 557 | D | B | — |
| 558 | D | C | — |
| 559 | C | C | — |
| 560 | D | C | — |
| 561 | D | C | — |
| 562 | D | D | — |
| 563 | D | B | — |
| 564 | B | B | — |
| 565 | B | B | — |
| 566 | B | B | — |
| 567 | B | B | — |
| 568 | B | B | — |
| 569 | D | D | — |
| 570 | D | D | — |
| 571 | D | C | — |
| 572 | C | C | — |
| 573 | D | D | — |
| 574 | D | D | — |
| 575 | D | C | — |
| 576 | D | D | — |
| 577 | C | B | — |
| 578 | B | B | — |
| 579 | D | D | — |
| 580 | D | C | — |
| 581 | D | C | — |
| 582 | D | D | — |
| 583 | C | D | — |
| 584 | C | A | — |
| 588 | C | A | — |
| 592 | C | C | — |
| 593 | D | D | — |
| 594 | D | D | — |
| 595 | C | D | — |
| 596 | C | C | — |
| 597 | D | D | — |
| 598 | D | C | — |
| 599 | C | C | — |
| 600 | D | D | — |
| 601 | D | D | — |
| 602 | D | D | — |
| 603 | C | B | — |
| 604 | C | D | — |
| 605 | D | B | — |
| 606 | D | D | B |
| 607 | B | A | A |
| 608 | B | A | — |
| 609 | D | B | — |
| 610 | D | D | — |
| 611 | D | D | — |
| 612 | D | B | — |
| 613 | C | C | — |
| 614 | C | C | — |
| 615 | C | B | — |
| 616 | C | C | — |
| 617 | D | C | — |
| 618 | C | B | — |
| 619 | C | B | — |
| 620 | B | A | — |
| 621 | C | B | — |
| 622 | B | A | — |
| 623 | B | A | — |
| 624 | D | D | — |
| 625 | B | A | — |
| 626 | D | D | — |
| 627 | D | D | — |
| 628 | B | A | — |
| 629 | B | A | — |
| 630 | C | C | — |
| 631 | C | C | — |
| 632 | B | A | — |
| 633 | D | B | — |
| 634 | A | A | — |
| 635 | C | B | A |
| 640 | B | A | — |
| 641 | B | B | — |
| 642 | C | C | — |
| 643 | C | B | — |
| 648 | D | C | — |
| 649 | D | C | — |
| 650 | A | A | — |
| 651 | D | D | — |
| 656 | D | D | — |
| 661 | C | C | — |
| 662 | D | D | — |
| 663 | C | C | — |
| 664 | C | C | — |
| 665 | B | B | — |
| 666 | C | B | — |
| 667 | D | D | — |
| 668 | C | B | — |
| 669 | C | C | — |
| 670 | C | D | — |
| 671 | D | B | — |
| 675 | A | A | — |
| 679 | C | B | — |
| 680 | D | C | — |
| 681 | B | B | — |
| 682 | B | A | — |
| 686 | D | D | — |
| 687 | D | C | — |
| 688 | A | A | — |
| 689 | A | A | — |
| 690 | A | A | — |
| 691 | D | B | — |
| 692 | C | A | — |
| 694 | D | D | — |
| 695 | C | C | — |
| 696 | D | D | — |
| 697 | B | A | — |
| 698 | C | A | — |
| 699 | B | A | — |
| 700 | B | A | — |
| 701 | D | B | — |
| 702 | D | C | — |
| 703 | D | D | — |
| 704 | B | A | — |
| 705 | B | A | — |
| 706 | B | B | — |
| 707 | C | A | — |
| 708 | D | D | — |
| 709 | C | B | — |
| 710 | C | C | — |
| 711 | D | C | — |
| 712 | A | A | — |
| 713 | D | B | — |
| 714 | B | A | — |
| 715 | D | D | — |
| 716 | C | C | — |
| 717 | C | D | — |
| 718 | C | A | — |
| 719 | C | A | — |
| 720 | B | A | — |
| 721 | A | A | — |
| 722 | D | D | — |
| 723 | B | A | — |
| 724 | B | A | — |
| 725 | D | D | — |
| 726 | D | C | — |
| 727 | B | A | — |
| 728 | D | B | — |
| 729 | C | D | — |
| 730 | D | B | — |
| 731 | D | B | — |
| 732 | D | A | — |
| 733 | D | D | — |
| 734 | D | D | — |
| 735 | D | C | — |
| 736 | D | D | — |
| 737 | D | D | — |
| 738 | D | A | — |
| 739 | C | A | — |
| 740 | C | D | — |
| 741 | D | A | — |
| 742 | D | B | — |
| 743 | A | A | — |
| 744 | C | A | — |
| 745 | B | A | — |
| 746 | B | B | — |
| 747 | B | A | — |
| 748 | C | A | — |
| 749 | B | A | — |
| 750 | A | A | — |
| 751 | D | D | — |
| 752 | D | D | — |
| 753 | D | D | — |
| 754 | D | D | — |
| 755 | C | B | — |
| 756 | B | A | — |
| 757 | D | A | — |
| 758 | D | A | — |
| 759 | C | A | — |
| 760 | D | D | — |
| 761 | D | B | — |
| 762 | D | A | — |
| 763 | C | A | — |
| 764 | C | A | — |
| 765 | B | A | — |
| 766 | A | A | — |
| 767 | C | A | — |
| 768 | A | A | — |
| 769 | B | A | — |
| 770 | B | B | — |
| 771 | C | B | — |
| 772 | D | C | — |
| 773 | C | C | — |
| 774 | D | D | — |
| 775 | D | D | — |
| 776 | D | D | — |
| 777 | C | C | — |
| 778 | D | D | — |
| 779 | D | B | — |
| 780 | B | A | — |
| 781 | D | D | — |
| 782 | D | B | — |
| 783 | D | A | — |
| 784 | B | A | — |
| 785 | B | A | — |
| 786 | C | B | — |
| 787 | D | C | — |
| 788 | D | B | — |
| 789 | B | A | — |
| 790 | C | A | — |
| 791 | D | D | — |
| 792 | C | C | — |
| 793 | D | C | — |
| 794 | D | B | — |
| 795 | D | D | — |
| 796 | D | D | — |
| 797 | D | D | — |
| 798 | C | C | — |
| 799 | D | C | — |
| 800 | B | A | — |
| 801 | C | A | — |
| 802 | C | B | — |
| 803 | D | D | — |
| 804 | D | D | — |
| 805 | D | C | — |
| 806 | D | C | — |
| 807 | D | D | — |
| 808 | D | D | — |
| 809 | D | B | — |
| 810 | C | A | — |
| 811 | D | D | — |
| 812 | D | D | — |
| 813 | D | C | — |
| 814 | D | B | — |
| 815 | D | D | — |
| 816 | D | C | — |
| 817 | D | C | — |
| 818 | D | D | — |
| 819 | C | C | — |
| 820 | D | C | — |
| 821 | C | B | — |
| 822 | D | B | — |
| 823 | D | C | — |
| 824 | D | C | — |
| 825 | D | C | — |
| 826 | D | D | — |
| 827 | C | B | — |
| 828 | C | B | — |
| 829 | D | D | — |
| 830 | D | D | — |
| 831 | B | B | — |
| 832 | C | C | — |
| 833 | C | B | — |
| 834 | D | D | — |
| 835 | D | D | — |
| 836 | D | B | — |
| 837 | C | A | — |
| 838 | D | C | — |
| 839 | C | B | — |
| 840 | C | C | — |
| 841 | D | D | — |
| 842 | D | D | — |
| 843 | D | D | — |
| 844 | D | D | — |
| 845 | D | D | — |
| 846 | B | B | — |
| 847 | B | A | — |
| 848 | B | B | — |
| 849 | B | B | — |
| 850 | D | D | — |
| 851 | D | D | — |
| 852 | D | B | — |
| 853 | D | D | — |
| 854 | D | B | — |
| 855 | C | C | — |
| 856 | C | C | — |
| 857 | D | D | — |
| 858 | C | B | — |
| 859 | C | B | — |
| 860 | D | D | — |
| 861 | C | B | — |
| 862 | B | B | — |
| 863 | D | D | — |
| 864 | D | C | — |
| 865 | D | C | — |
| 866 | D | D | — |
| 867 | D | D | — |
| 868 | D | D | — |
| 869 | D | D | — |
| 870 | D | D | — |
| 871 | D | C | — |
| 872 | D | D | — |
| 873 | D | D | — |
| 874 | D | D | — |
| 875 | D | D | — |
| 876 | C | C | — |
| 877 | D | D | — |
| 878 | D | D | — |
| 879 | D | C | — |
| 880 | D | C | — |
| 881 | C | C | — |
| 882 | D | B | — |
| 883 | D | C | — |
| 884 | D | C | — |
| 885 | C | C | — |

### Example 333: Investigating Effect of Exemplary Compounds on Cell Viability

Compounds described herein were screened for toxicity in K562 (human chronic myelogenous leukemia) and SH-SY5Y (human neuroblastoma) cells using a Cell Titer Glo 2.0 assay.

### Materials

Promega CellTiter-Glo® 2.0 Cell Viability Assay (cat #G9241)
Corning 384-well TC-treated microplates (cat #3570)

### Description

Cells were plated at 500 cells/well (K562 cells) in 45 μL of IMDM supplemented with 10% FBS in a 384-well opaque plate. Wells containing only medium were used as a blank control. Test compounds (e.g., compounds of Formula (I) or (II) were first serially diluted in DMSO then diluted 1:100 with IMDM+10% FBS. The final concentration of DMSO was 0.1% in each well. The cells were incubated for 72 hours at 37° C. and 5% CO₂ before assaying with Cell Titer Glo 2.0 reagent.

TABLE 6
Effect on Cell Viability by Exemplary Compounds

| | Viability | Viability |
|---|---|---|
| | K562.3 | SHSY5Y.3 |
| Cmpd | GI50 | GI50 |
| No. | (nM) | (nM) |
| 100 | B | C |
| 101 | B | C |
| 104 | B | B |
| 107 | B | C |
| 108 | C | C |
| 112 | B | C |
| 113 | C | C |
| 115 | B | C |
| 118 | C | D |
| 119 | C | C |
| 122 | C | C |
| 124 | C | C |
| 125 | B | B |
| 127 | C | C |
| 128 | B | C |
| 129 | B | C |
| 132 | C | C |
| 135 | B | B |
| 136 | B | B |
| 137 | C | C |
| 140 | B | C |
| 143 | B | B |
| 144 | B | B |
| 146 | D | D |
| 149 | C | C |
| 152 | C | D |
| 153 | B | B |
| 154 | C | B |
| 155 | C | B |
| 157 | C | C |
| 160 | D | D |
| 163 | B | B |
| 166 | C | C |
| 169 | B | C |
| 170 | A | B |
| 171 | C | D |
| 172 | D | D |
| 173 | A | A |
| 174 | B | B |
| 175 | A | C |
| 176 | B | B |
| 177 | C | B |
| 178 | C | C |
| 179 | A | A |
| 180 | A | A |
| 181 | B | B |
| 182 | A | B |
| 183 | A | B |
| 184 | B | B |
| 185 | B | B |
| 186 | B | B |
| 187 | B | B |
| 188 | C | C |
| 189 | B | B |
| 190 | C | D |
| 191 | B | B |
| 192 | B | B |
| 193 | B | C |
| 194 | C | C |
| 195 | C | C |
| 196 | B | C |
| 197 | A | A |
| 198 | D | D |
| 199 | B | C |
| 200 | A | B |
| 201 | C | C |
| 202 | A | B |
| 203 | A | B |
| 204 | A | B |
| 205 | A | B |
| 206 | A | B |
| 207 | A | A |
| 208 | A | A |
| 209 | A | A |
| 210 | A | A |
| 211 | B | B |
| 212 | C | C |
| 213 | C | C |
| 214 | D | D |
| 215 | D | D |
| 220 | C | C |
| 221 | C | C |
| 225 | C | C |
| 230 | D | D |
| 237 | C | C |
| 242 | D | D |
| 243 | D | D |
| 247 | D | C |
| 248 | D | D |
| 249 | C | C |
| 250 | D | D |
| 251 | D | D |
| 252 | B | C |
| 257 | D | D |
| 261 | D | D |
| 263 | B | C |
| 265 | B | C |
| 266 | C | C |
| 267 | D | D |
| 268 | B | B |
| 275 | B | C |
| 277 | C | C |
| 279 | D | D |
| 284 | C | D |
| 285 | B | C |
| 288 | B | B |
| 291 | B | B |
| 294 | B | B |
| 297 | B | C |
| 298 | B | C |
| 299 | D | D |
| 300 | D | C |
| 301 | D | D |
| 302 | C | D |
| 303 | C | C |
| 304 | B | C |
| 305 | D | D |
| 306 | A | C |
| 307 | — | C |
| 308 | — | C |
| 309 | — | D |
| 310 | D | D |
| 311 | D | D |
| 312 | C | C |
| 313 | C | C |
| 314 | B | B |
| 315 | B | C |
| 316 | B | C |
| 317 | C | D |
| 318 | D | D |
| 319 | C | D |
| 320 | B | B |
| 321 | A | A |
| 322 | B | C |
| 323 | A | C |
| 324 | A | C |
| 325 | A | B |
| 326 | B | C |
| 327 | A | B |
| 328 | A | B |
| 329 | B | C |
| 330 | C | D |
| 331 | A | C |
| 332 | A | C |
| 333 | D | D |
| 334 | A | B |
| 335 | A | A |
| 336 | D | D |
| 337 | C | D |
| 338 | A | B |
| 339 | B | B |
| 340 | C | D |
| 341 | C | D |
| 342 | B | C |
| 343 | A | A |
| 344 | B | C |
| 345 | B | C |
| 346 | C | C |
| 347 | A | C |
| 348 | B | C |
| 349 | A | B |
| 350 | B | C |
| 351 | C | C |
| 352 | A | B |
| 353 | B | D |
| 354 | B | C |
| 355 | A | B |
| 356 | B | C |
| 357 | A | B |
| 358 | B | C |
| 359 | B | B |
| 360 | C | C |
| 361 | B | C |
| 362 | B | C |
| 363 | C | D |
| 364 | B | C |
| 365 | C | D |
| 366 | D | D |
| 367 | B | B |
| 368 | B | B |
| 369 | A | B |
| 370 | B | C |
| 371 | B | C |
| 375 | A | B |
| 376 | C | C |
| 377 | A | A |
| 378 | A | B |
| 379 | C | D |
| 380 | C | D |
| 381 | C | C |
| 382 | A | A |
| 383 | A | C |
| 384 | C | D |
| 385 | B | C |
| 386 | B | D |
| 387 | A | C |
| 388 | A | C |
| 389 | A | A |
| 390 | B | C |
| 391 | A | B |
| 392 | C | D |
| 393 | B | C |
| 394 | B | D |
| 395 | A | B |
| 396 | A | A |
| 397 | A | C |
| 398 | A | B |
| 399 | B | B |
| 400 | B | D |
| 401 | C | D |
| 402 | C | D |
| 403 | C | B |
| 404 | A | B |
| 405 | A | A |
| 406 | A | B |
| 407 | B | C |
| 408 | A | B |
| 409 | B | D |
| 410 | D | D |
| 411 | D | D |
| 412 | C | C |
| 413 | C | C |
| 414 | C | D |
| 415 | A | B |
| 416 | D | D |
| 417 | A | B |
| 418 | B | B |
| 419 | A | C |
| 420 | C | D |
| 421 | D | C |
| 422 | A | B |
| 423 | B | C |
| 424 | D | D |
| 425 | B | B |
| 426 | B | C |
| 427 | D | D |
| 428 | C | C |
| 429 | C | C |
| 431 | A | B |
| 432 | A | A |
| 433 | A | A |
| 434 | B | C |
| 435 | C | C |
| 436 | B | C |
| 437 | D | C |
| 438 | D | D |
| 439 | D | D |
| 440 | A | B |
| 441 | C | C |
| 442 | C | C |
| 443 | A | B |
| 444 | B | B |
| 445 | D | D |
| 446 | C | C |
| 447 | C | C |
| 449 | B | D |
| 450 | B | C |
| 451 | A | B |
| 452 | B | C |
| 453 | B | C |
| 454 | D | D |
| 455 | B | D |
| 456 | A | C |
| 457 | B | C |
| 458 | B | C |
| 459 | A | C |
| 460 | A | C |
| 461 | B | B |
| 462 | B | C |
| 463 | B | B |
| 464 | B | B |
| 465 | A | B |
| 466 | D | C |
| 467 | A | B |
| 468 | A | C |
| 469 | A | B |
| 470 | B | B |
| 471 | B | C |
| 472 | B | C |
| 473 | B | D |
| 474 | C | C |
| 475 | B | C |
| 476 | C | D |
| 477 | B | B |
| 478 | B | C |
| 479 | B | C |
| 481 | C | C |
| 482 | D | C |
| 483 | C | B |
| 484 | C | C |
| 485 | B | B |
| 486 | C | C |
| 487 | C | C |
| 488 | C | D |
| 489 | D | D |
| 490 | C | C |
| 491 | C | C |
| 492 | C | D |
| 493 | B | C |
| 494 | C | D |
| 495 | B | B |
| 496 | C | D |
| 497 | C | C |
| 498 | C | D |
| 499 | C | C |
| 500 | D | D |
| 501 | B | B |
| 502 | B | B |
| 505 | B | B |
| 506 | C | D |
| 507 | C | C |
| 508 | C | C |
| 509 | C | D |
| 510 | C | C |
| 511 | D | D |
| 512 | C | C |
| 513 | C | D |
| 514 | B | D |
| 515 | B | C |
| 518 | B | C |
| 519 | B | B |
| 520 | C | D |
| 521 | C | D |
| 522 | C | C |
| 523 | A | C |
| 524 | A | B |
| 525 | C | D |
| 526 | C | C |
| 527 | B | B |
| 528 | B | B |
| 529 | B | C |
| 530 | C | D |
| 531 | B | C |
| 532 | B | B |
| 533 | B | B |
| 534 | C | D |
| 535 | C | C |
| 536 | D | D |
| 537 | C | D |
| 538 | B | C |
| 539 | D | D |
| 540 | B | C |
| 541 | B | C |
| 542 | C | D |
| 543 | B | C |
| 544 | B | B |
| 545 | C | C |
| 546 | B | C |
| 547 | C | D |
| 548 | C | C |
| 549 | C | D |
| 553 | B | C |
| 557 | C | C |
| 558 | C | B |
| 559 | C | C |
| 560 | D | D |
| 561 | D | D |
| 562 | C | D |
| 563 | B | D |
| 564 | B | B |
| 565 | B | B |
| 566 | B | C |
| 567 | B | C |
| 568 | B | C |
| 569 | C | C |
| 570 | D | D |
| 571 | B | C |
| 572 | B | C |
| 573 | D | D |
| 574 | B | C |
| 575 | B | C |
| 576 | C | C |
| 577 | B | C |
| 578 | B | B |
| 579 | D | D |
| 580 | B | B |
| 581 | B | C |
| 582 | B | C |
| 583 | C | D |
| 584 | B | B |
| 588 | B | B |
| 592 | C | C |
| 593 | D | D |
| 594 | C | D |
| 595 | C | C |
| 596 | C | C |
| 597 | C | C |
| 598 | B | C |
| 599 | B | C |
| 600 | C | C |
| 601 | D | D |
| 602 | D | D |
| 603 | B | C |
| 604 | B | C |
| 605 | B | C |
| 606 | B | B |
| 607 | A | B |
| 608 | A | B |
| 609 | A | D |
| 610 | D | D |
| 611 | D | D |
| 612 | B | C |
| 613 | C | D |
| 614 | C | D |
| 615 | B | C |
| 616 | C | C |
| 617 | B | C |
| 618 | B | C |
| 619 | B | C |
| 620 | B | B |
| 621 | B | B |
| 622 | A | B |
| 623 | A | B |
| 624 | D | D |
| 625 | A | B |
| 626 | D | D |
| 627 | D | D |
| 628 | B | B |
| 629 | B | B |
| 630 | B | C |
| 631 | B | C |
| 632 | B | C |
| 633 | B | D |
| 634 | B | B |
| 635 | B | C |
| 640 | B | B |
| 641 | B | B |
| 642 | — | C |
| 643 | — | C |
| 648 | — | D |
| 649 | — | C |
| 650 | — | B |
| 651 | — | D |
| 656 | D | D |
| 661 | C | C |
| 662 | C | C |
| 663 | C | C |
| 664 | C | C |
| 665 | B | B |
| 666 | B | C |
| 667 | B | C |
| 668 | B | B |
| 669 | B | C |
| 670 | C | D |
| 671 | B | C |
| 675 | A | A |
| 679 | C | C |
| 680 | C | D |
| 681 | B | B |
| 682 | A | A |
| 686 | C | D |
| 687 | B | D |
| 688 | A | A |
| 689 | A | A |
| 690 | A | B |
| 691 | C | C |
| 692 | A | C |
| 694 | C | D |
| 695 | C | C |
| 696 | B | C |
| 697 | A | A |
| 698 | B | B |
| 699 | A | A |
| 700 | B | B |
| 701 | B | D |
| 702 | C | D |
| 703 | C | D |
| 704 | A | B |
| 705 | A | A |
| 706 | B | B |
| 707 | B | B |
| 708 | B | C |
| 709 | A | C |
| 710 | B | C |
| 711 | B | C |
| 712 | A | A |
| 713 | B | B |
| 714 | A | A |
| 715 | B | B |
| 716 | C | C |
| 717 | C | C |
| 718 | A | A |
| 719 | B | B |
| 720 | A | A |
| 721 | A | A |
| 722 | C | C |
| 723 | A | B |
| 724 | B | C |
| 725 | B | D |
| 726 | C | C |
| 727 | A | A |
| 728 | C | C |
| 729 | C | C |
| 730 | C | C |
| 731 | B | C |
| 732 | B | B |
| 733 | C | C |
| 734 | C | D |
| 735 | B | D |
| 736 | C | C |
| 737 | C | C |
| 738 | B | B |
| 739 | B | B |
| 740 | B | C |
| 741 | B | A |
| 742 | B | C |
| 743 | A | A |
| 744 | B | B |
| 745 | B | B |
| 746 | B | B |
| 747 | B | B |
| 748 | B | B |
| 749 | B | B |
| 750 | A | B |
| 751 | D | D |
| 752 | C | D |
| 753 | C | D |
| 754 | C | D |
| 755 | B | B |
| 756 | A | A |
| 757 | A | B |
| 758 | B | B |
| 759 | A | B |
| 760 | C | C |
| 761 | B | C |
| 762 | B | C |
| 763 | A | A |
| 764 | B | B |
| 765 | B | B |
| 766 | A | A |
| 767 | B | B |
| 768 | A | B |
| 769 | B | B |
| 770 | B | B |
| 771 | B | B |
| 772 | B | C |
| 773 | C | C |
| 774 | C | D |
| 775 | C | D |
| 776 | B | C |
| 777 | C | C |
| 778 | B | D |
| 779 | B | D |
| 780 | A | A |
| 781 | C | C |
| 782 | A | C |
| 783 | A | B |
| 784 | A | A |
| 785 | A | A |
| 786 | C | C |
| 787 | B | C |
| 788 | B | C |
| 789 | B | B |
| 790 | B | B |
| 791 | C | C |
| 792 | C | C |
| 793 | B | C |
| 794 | B | C |
| 795 | C | C |
| 796 | C | C |
| 797 | D | D |
| 798 | C | C |
| 799 | C | C |
| 800 | A | A |
| 801 | A | B |
| 802 | B | C |
| 803 | C | C |
| 804 | C | C |
| 805 | B | C |
| 806 | B | C |
| 807 | C | C |
| 808 | C | C |
| 809 | C | B |
| 810 | A | A |
| 811 | D | D |
| 812 | C | C |
| 813 | B | D |
| 814 | B | C |
| 815 | C | C |
| 816 | B | D |
| 817 | C | D |
| 818 | C | D |
| 819 | C | C |
| 820 | C | D |
| 821 | B | C |
| 822 | B | B |
| 823 | B | C |
| 824 | B | C |
| 825 | C | D |
| 826 | C | D |
| 827 | B | C |
| 828 | B | C |
| 829 | C | D |
| 830 | C | D |
| 831 | B | B |
| 832 | B | C |
| 833 | B | C |
| 834 | C | D |
| 835 | D | D |
| 836 | B | C |
| 837 | A | B |
| 838 | B | C |
| 839 | B | C |
| 840 | C | D |
| 841 | D | D |
| 842 | D | C |
| 843 | D | D |
| 844 | D | C |
| 845 | D | C |
| 846 | B | B |
| 847 | B | B |
| 848 | B | B |
| 849 | B | B |
| 850 | D | D |
| 851 | D | D |
| 852 | B | C |
| 853 | D | D |
| 854 | B | C |
| 855 | C | C |
| 856 | C | C |
| 857 | D | D |
| 858 | B | B |
| 859 | B | C |
| 860 | C | C |
| 861 | A | C |
| 862 | B | B |
| 863 | D | D |
| 864 | C | D |
| 865 | C | D |
| 866 | D | D |
| 867 | D | D |
| 868 | D | C |
| 869 | D | D |
| 870 | D | B |
| 871 | C | D |
| 872 | C | D |
| 873 | D | D |
| 874 | C | D |
| 875 | D | D |
| 876 | C | C |
| 877 | C | D |
| 878 | D | D |
| 879 | B | C |
| 880 | B | C |
| 881 | B | C |
| 882 | B | C |
| 883 | C | D |
| 884 | B | D |
| 885 | C | C |

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present invention, as defined in the following claims.

1. A compound of Formula (I-c): 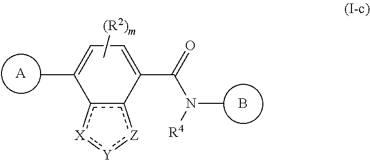
or a pharmaceutically acceptable salt, solvate, hydrate, tautomer, or stereoisomer thereof, wherein:
A is a nitrogen-containing heterocyclyl optionally substituted with one or more R¹;
B is a nitrogen-containing heteroaryl and optionally substituted with one or more R¹;
X, Y, and Z are each independently C(R^{3a}), C(R^{3a})(R^{3b}), N, N(R^{3c}), S, or O, wherein at least one of X, Y, and Z is N, N(R^{3c}), or O, and the bonds in the ring comprising X, Y, and Z may be single or double bonds as valency permits;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ heteroalkylene-aryl, heteroaryl, C₁-C₆ alkylene-heteroaryl, C₁-C₆ heteroalkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶;
each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D};
R^{3a} and R^{3b} are each independently hydrogen, C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —C(O)R^{D}, or —C(O)OR^{D}; or
each of R^{3a} and R^{3b}, together with the carbon atom to which they are attached, form an oxo group;
R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸;
each R⁴ is independently hydrogen, C₁-C₆-alkyl, C₁-C₆-heteroalkyl, or C₁-C₆-haloalkyl;
each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹;
each R⁸ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, —SR^{E}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹²;
each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R¹² is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A},
each R^{A} is independently hydrogen, C₁-C₆ alkyl, C₂-C₆ alkenyl, C₂-C₆ alkynyl, C₁-C₆ haloalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D};
each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, —C(O)R^{D}, or —S(O)ₓR^{D}; or
R^{B} and R^{C} together with the atom to which they are attached form a 3-7-membered heterocyclyl ring optionally substituted with one or more R⁷;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R^{A1} is hydrogen or C₁-C₆-alkyl;
m is 0, 1, or 2; and
x is 0, 1, or 2.

2. The compound of claim 1, wherein each of B is monocyclic or bicyclic heteroaryl, each of which is optionally substituted with one or more R¹.

3. The compound of any one of the preceding claims, wherein B is monocyclic heteroaryl optionally substituted with one or more R¹.

4. The compound of any one of the preceding claims, wherein B is bicyclic heteroaryl optionally substituted with one or more R¹.

5. The compound of any one of the preceding claims, wherein B is a 5-10 membered heteroaryl optionally substituted with one or more R¹.

6. The compound of any one of the preceding claims, wherein B is a bicyclic nitrogen-containing heteroaryl containing at least two nitrogen atoms optionally substituted with one or more R¹.

7. The compound of any one of the preceding claims, wherein B is selected from 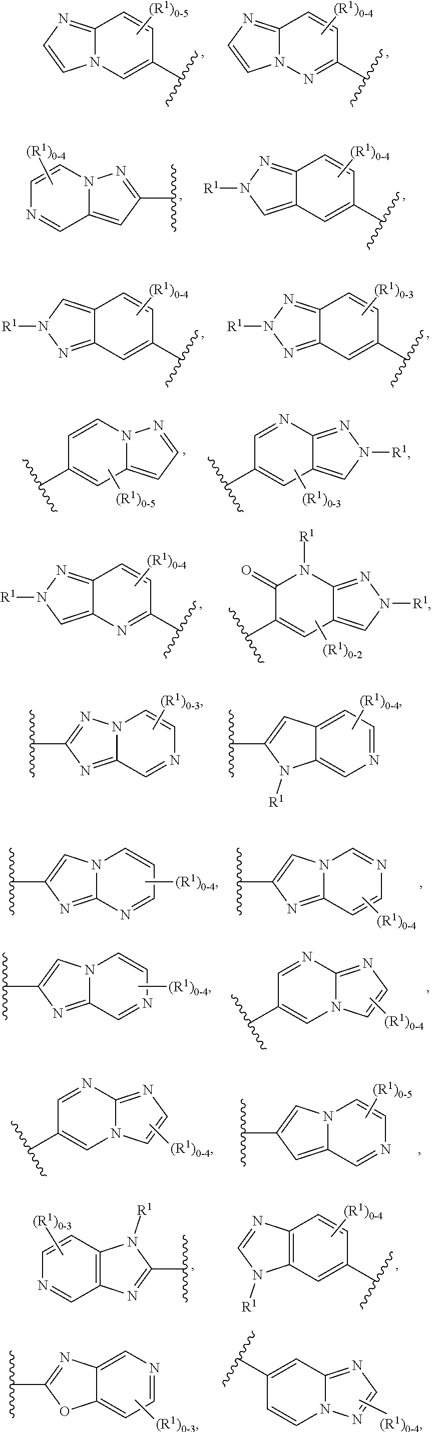 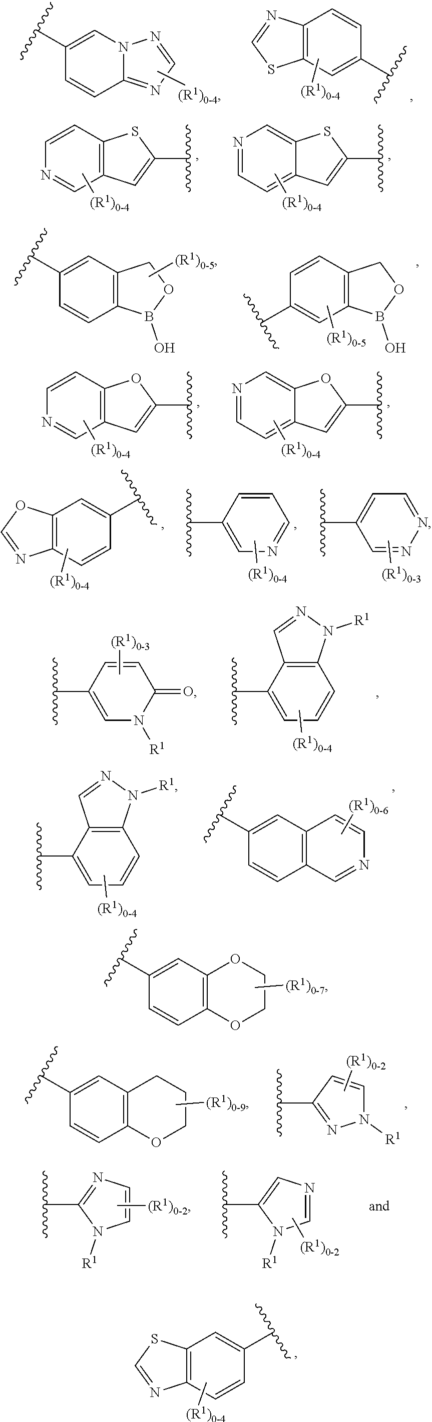
wherein R¹ is as described in claim 1.

8. The compound of any one of the preceding claims, wherein B is selected from 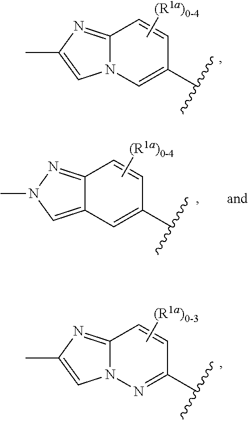
and each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

9. The compound of any one of the preceding claims, wherein B is 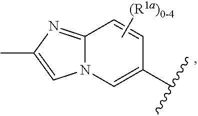
and each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

10. The compound of any one of the preceding claims, wherein B is 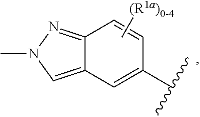
and each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

11. The compound of any one of the preceding claims, wherein B is selected from

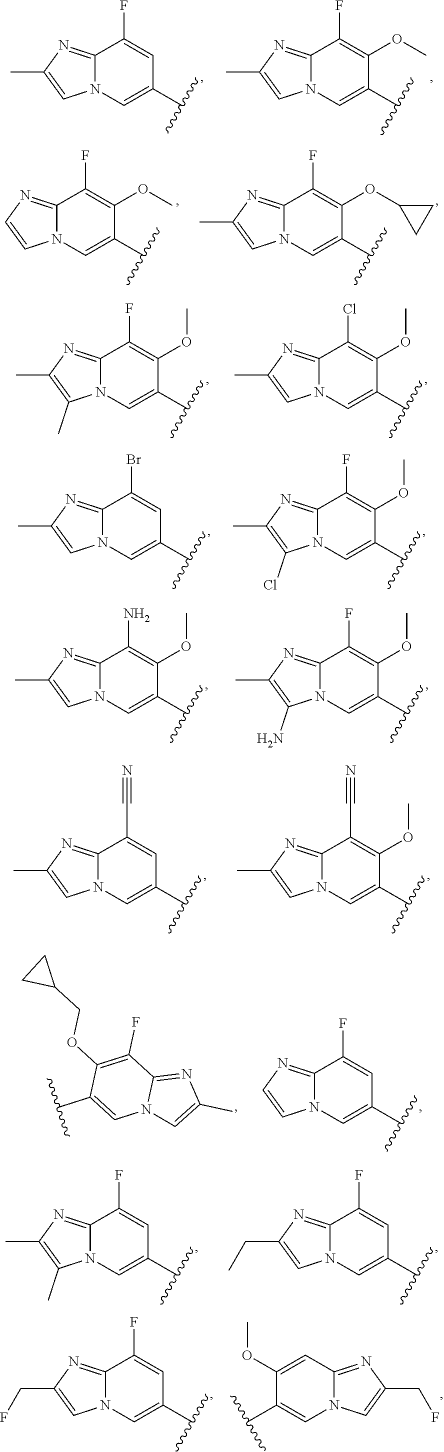

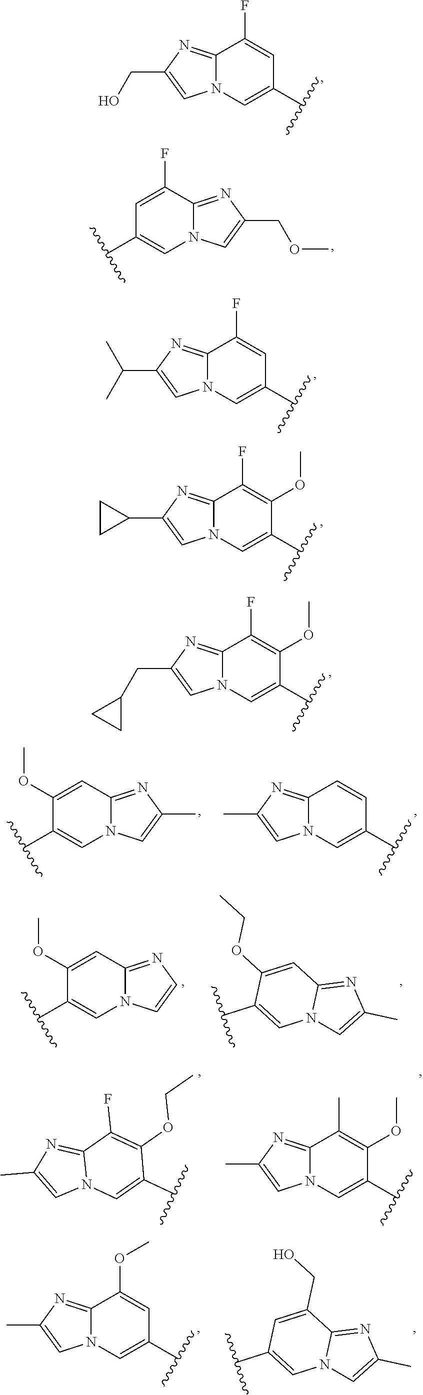

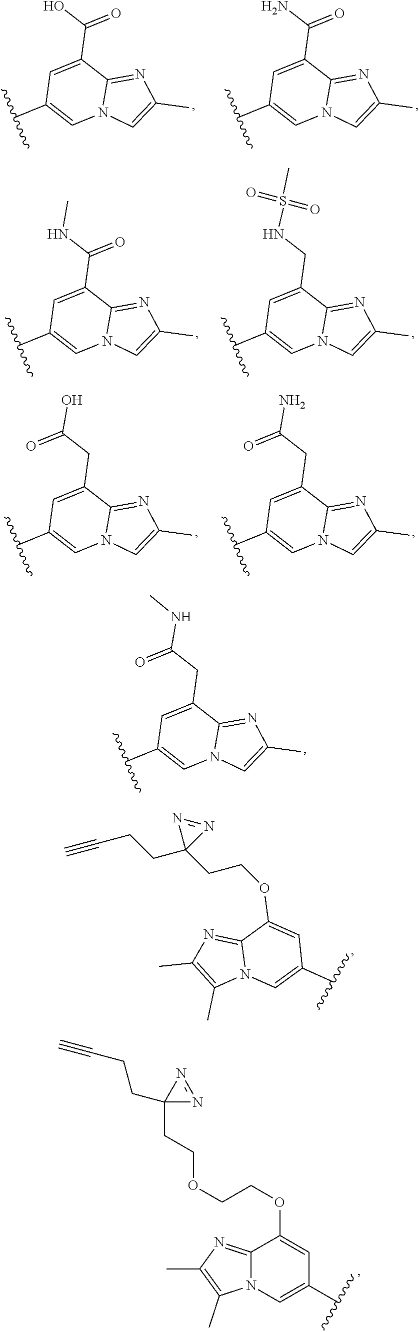

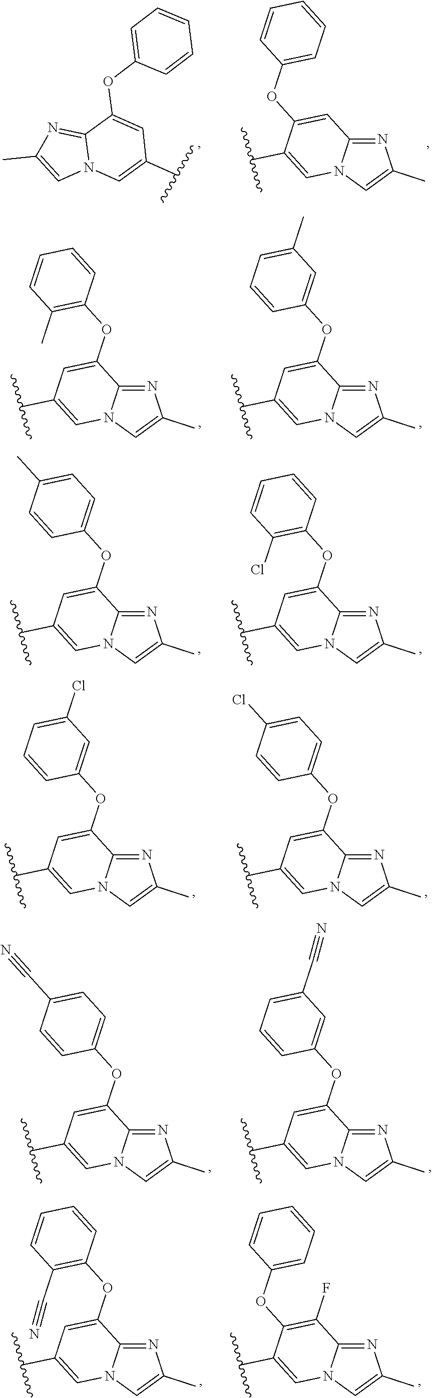

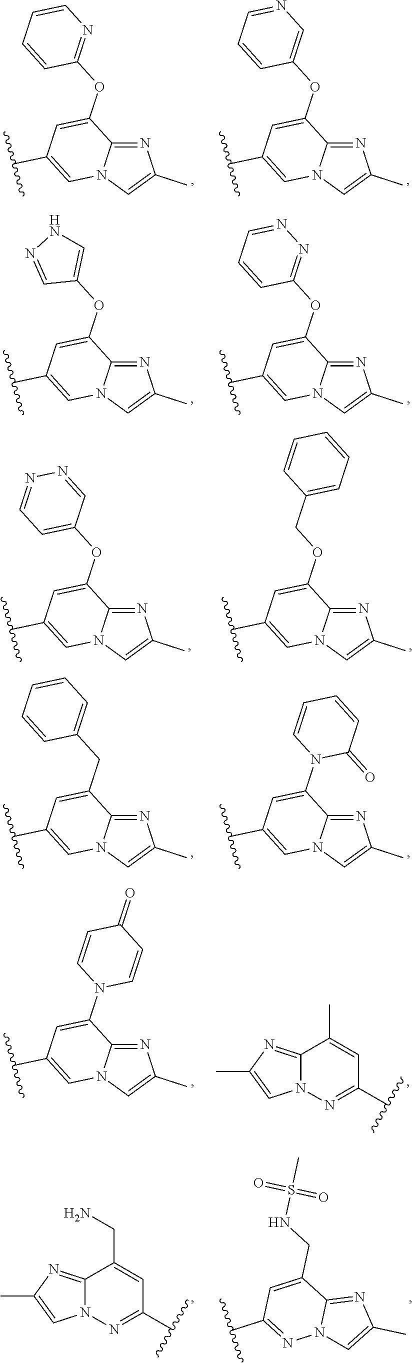

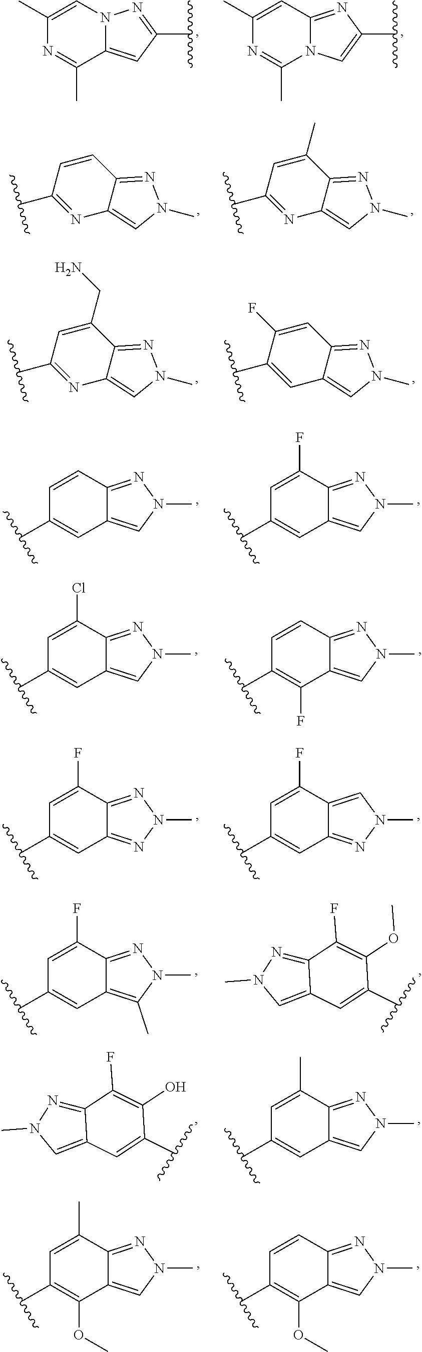

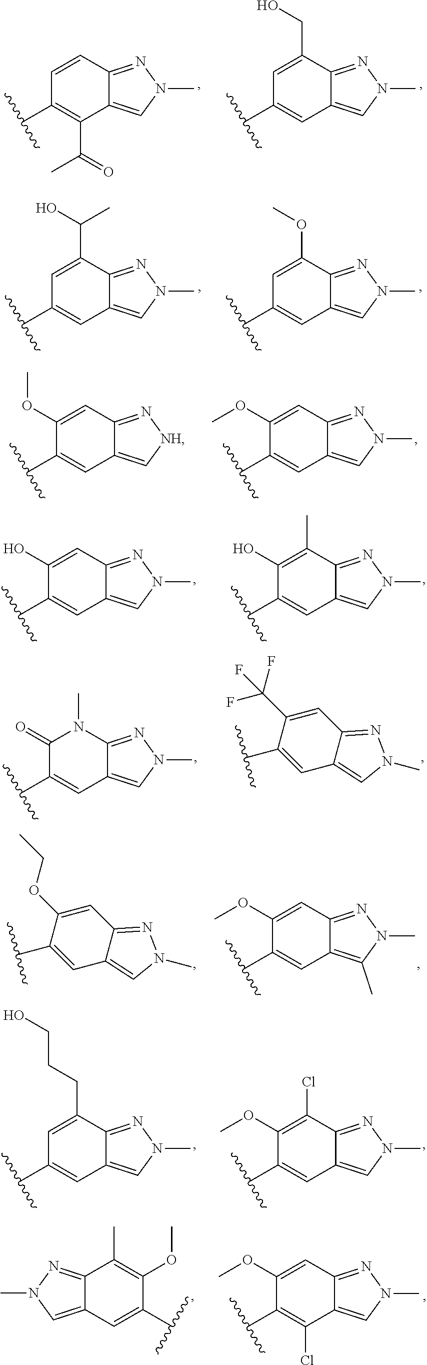

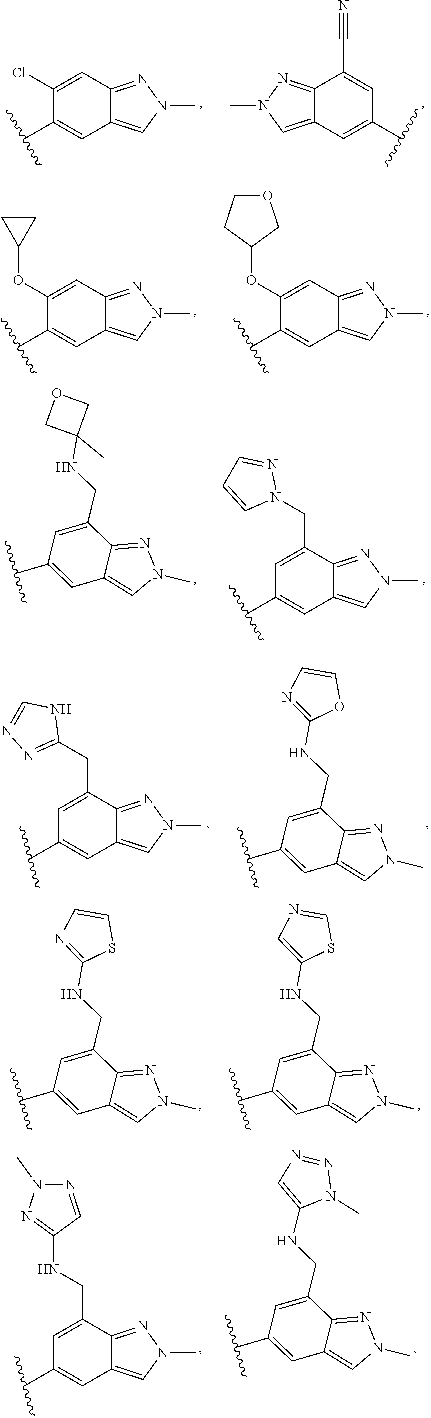

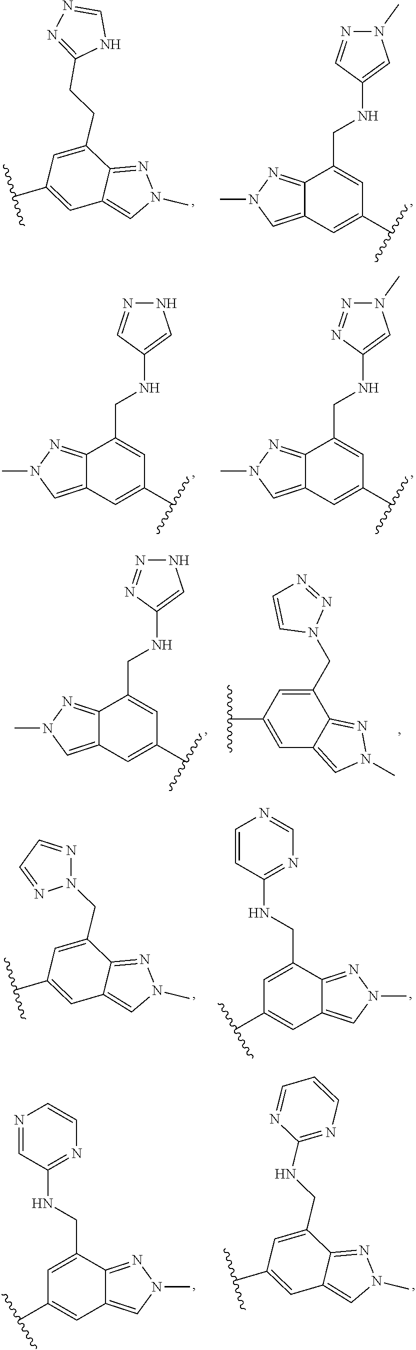

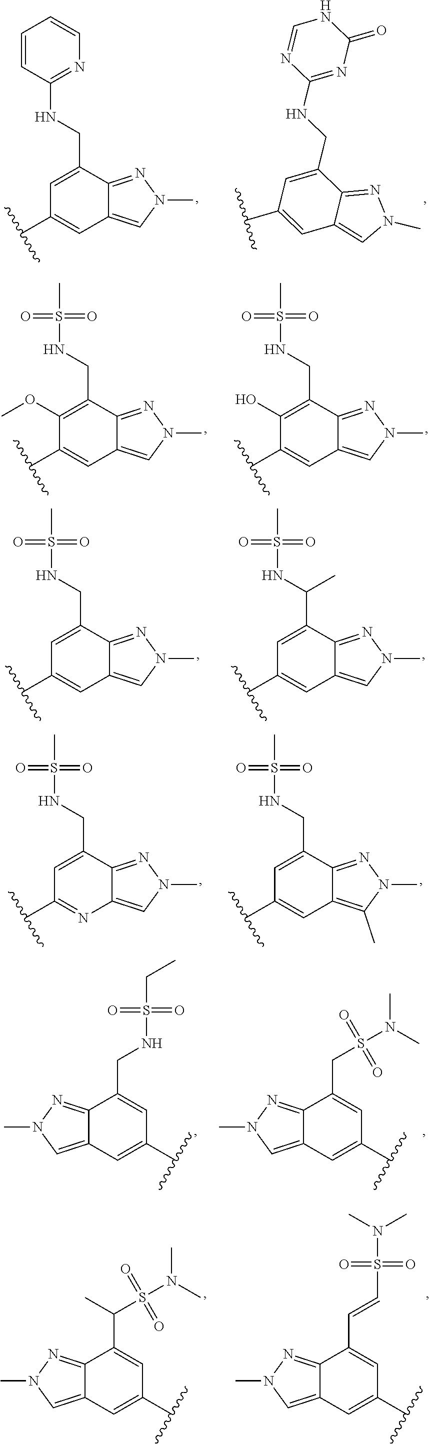

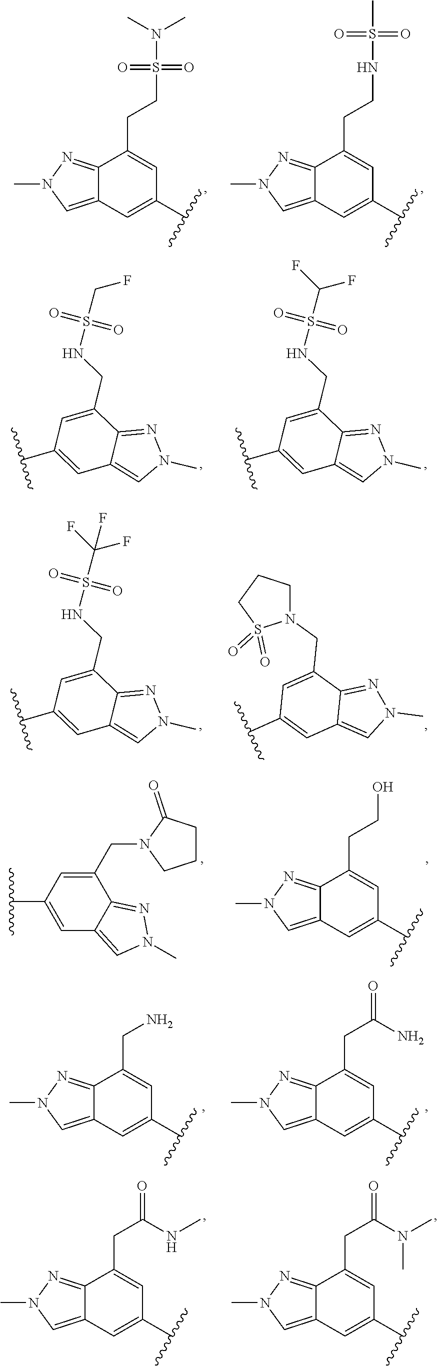

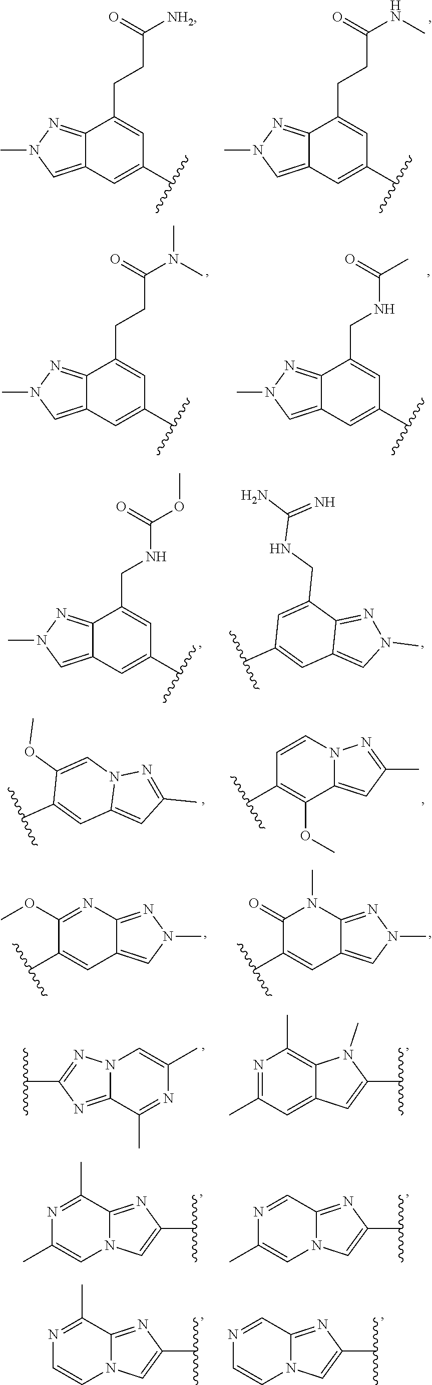

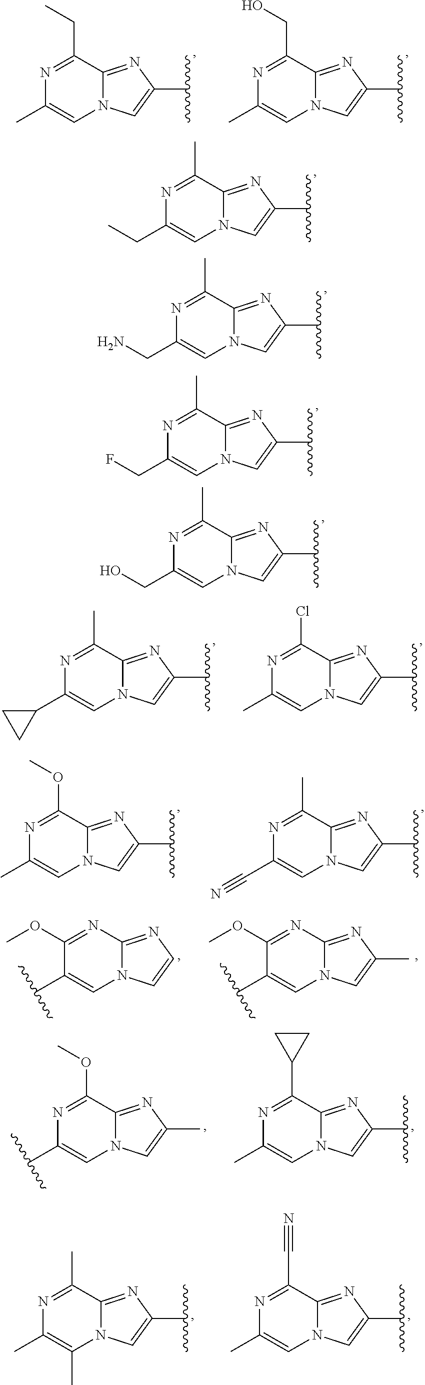

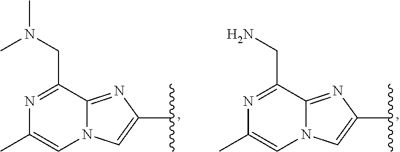 [SMILES: *c1cn2cc(C)nc(CN(C)C)c2n1 ; *c1cn2cc(C)nc(CN)c2n1]

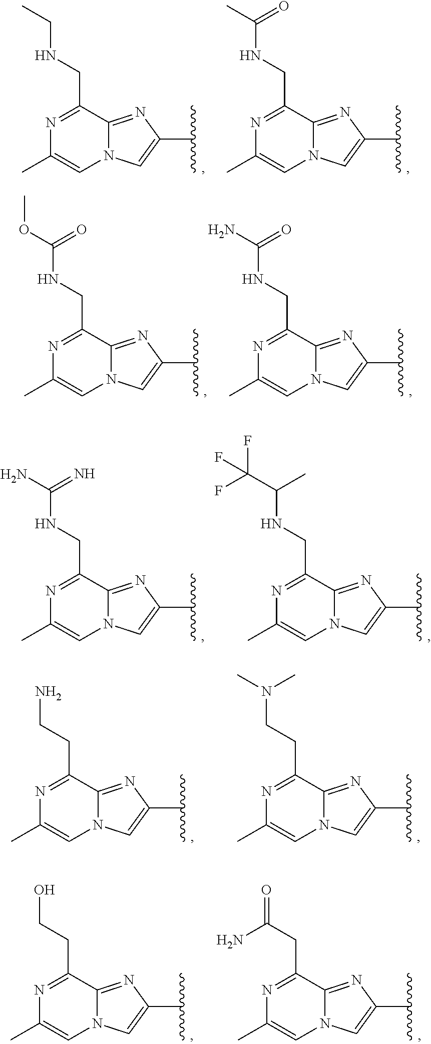

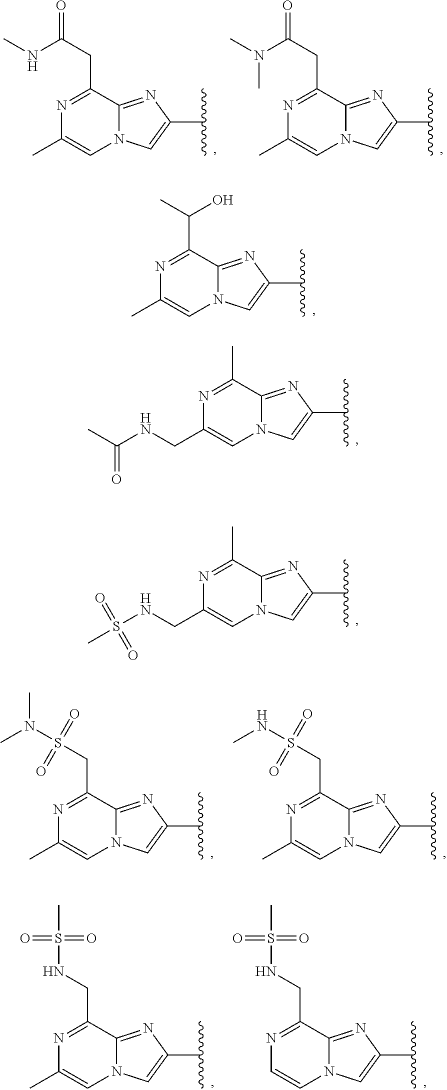

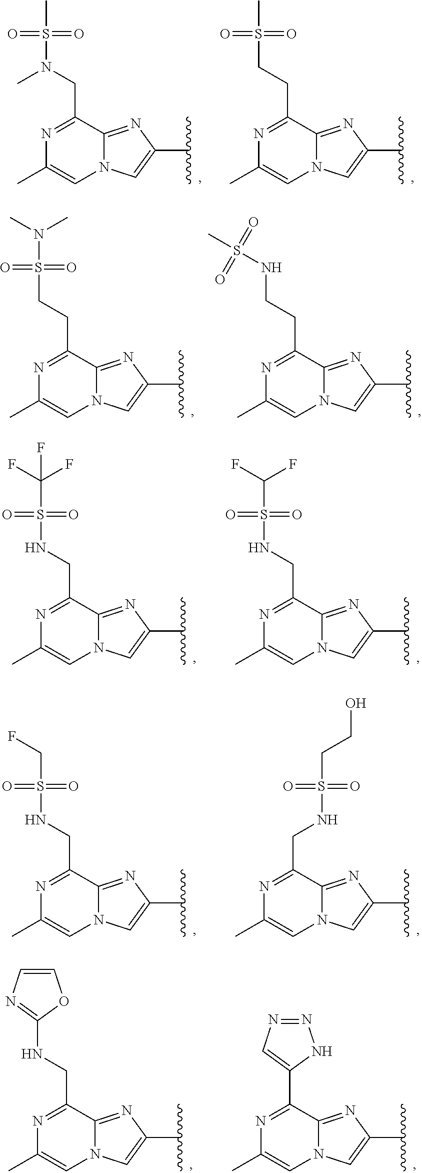

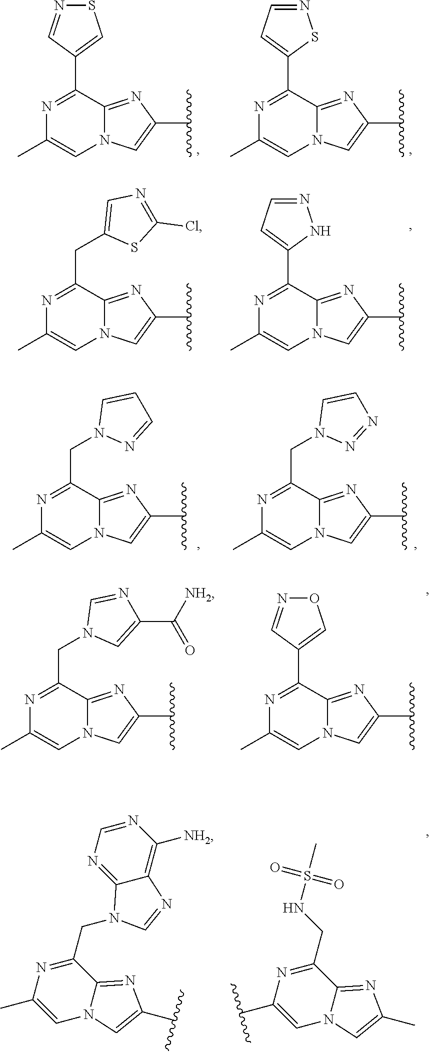

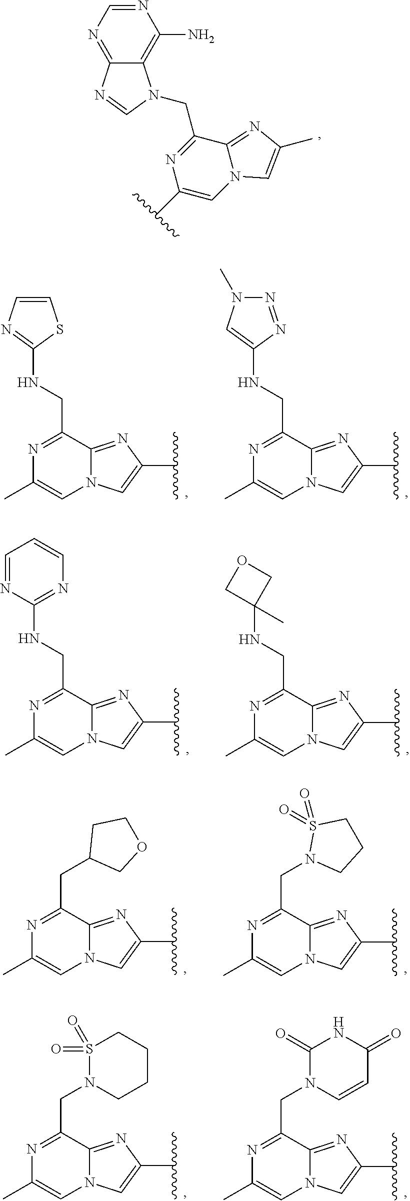

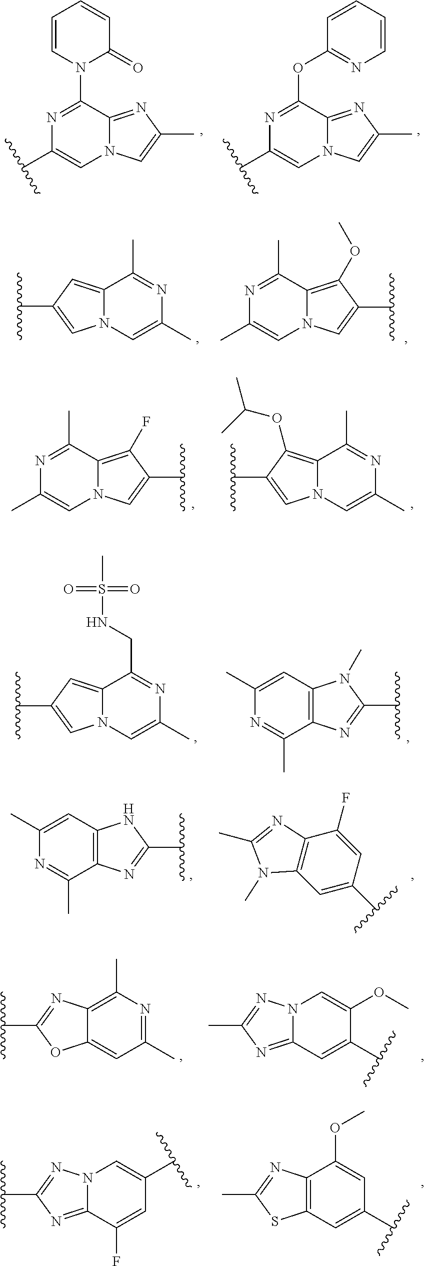

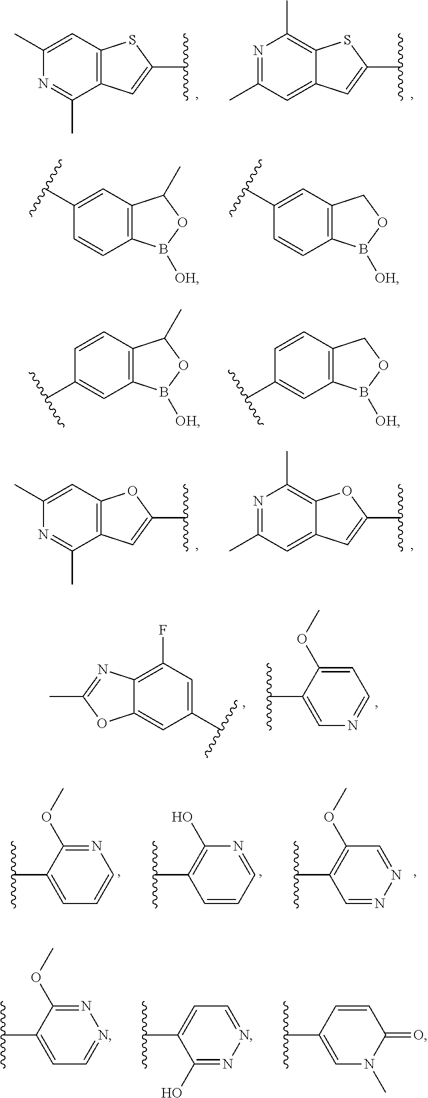

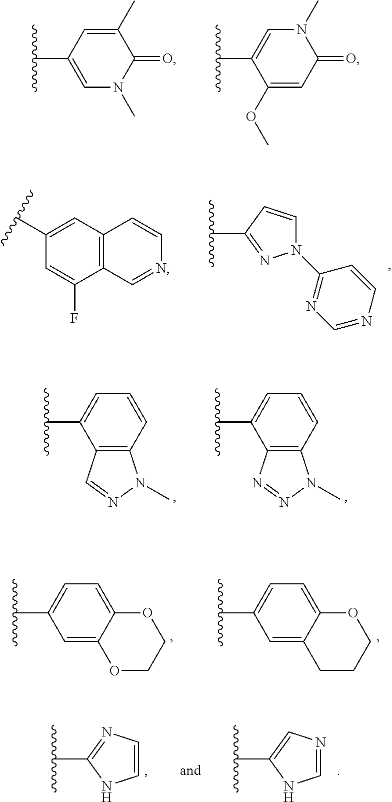

12. The compound of any one of the preceding claims, wherein B is selected from

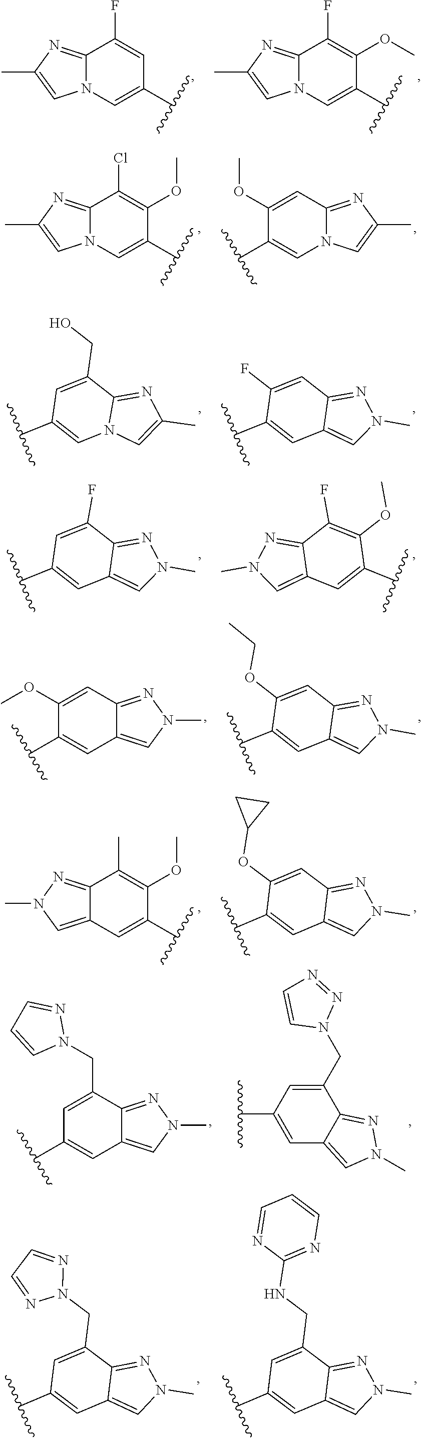

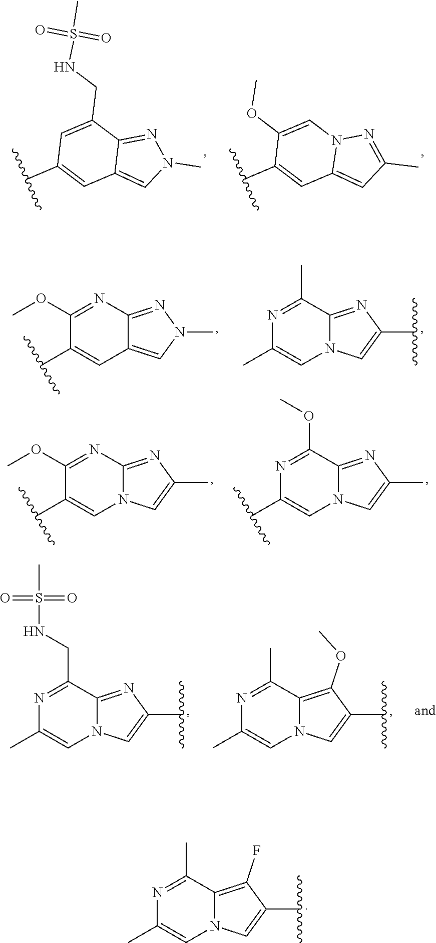

13. The compound of any one of the preceding claims, wherein B is selected from

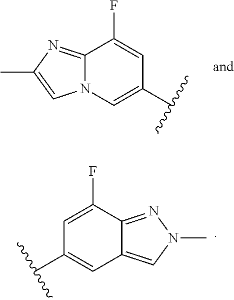 [SMILES: *c1cc(F)c2nc(C)cn2c1 ; *c1cc(F)c2nn(C)cc2c1]

14. The compound of any one of the preceding claims, wherein A is monocyclic heterocyclyl or bicyclic heterocyclyl, each of which is optionally substituted with one or more R¹.

15. The compound of any one of the preceding claims, wherein A is a monocyclic nitrogen-containing heterocyclyl optionally substituted with one or more R¹.

16. The compound of any one of the preceding claims, wherein A is a 4-8 membered heterocyclyl optionally substituted with one or more R¹.

17. The compound of any one of the preceding claims, wherein A is selected from 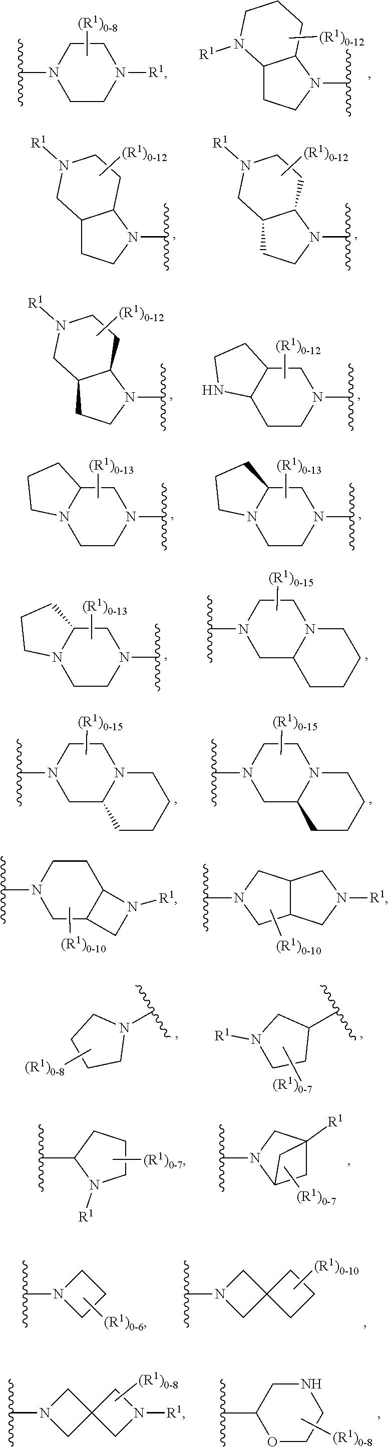 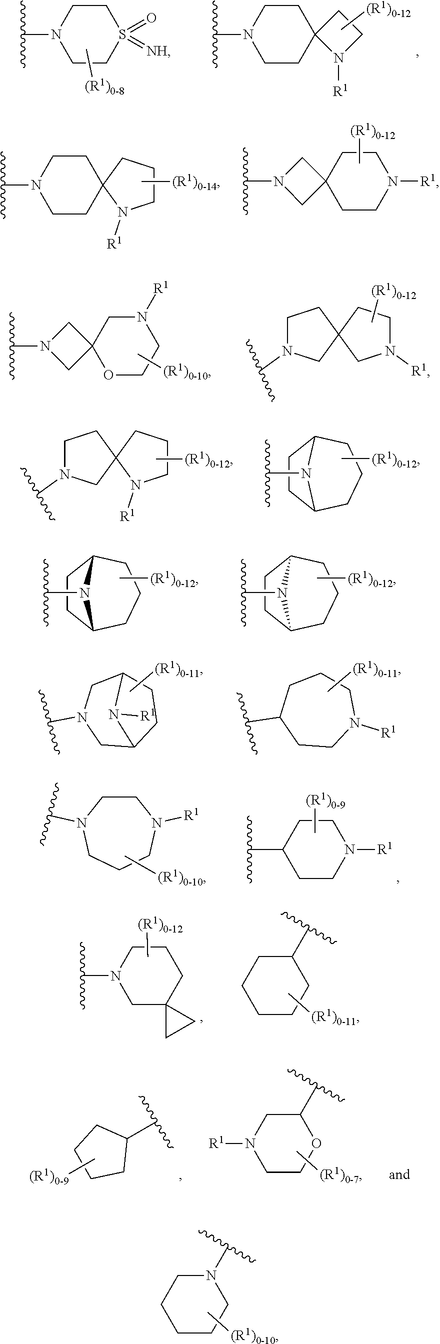
and R¹ is as described in claim 1.

18. The compound of any one of the preceding claims, wherein A is selected from 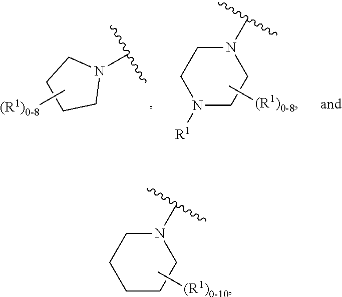
and R¹ is as described in claim 1.

19. The compound of any one of the preceding claims, wherein A is 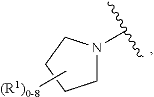
and R¹ is as described in claim 1.

20. The compound of any one of the preceding claims, wherein A is selected from

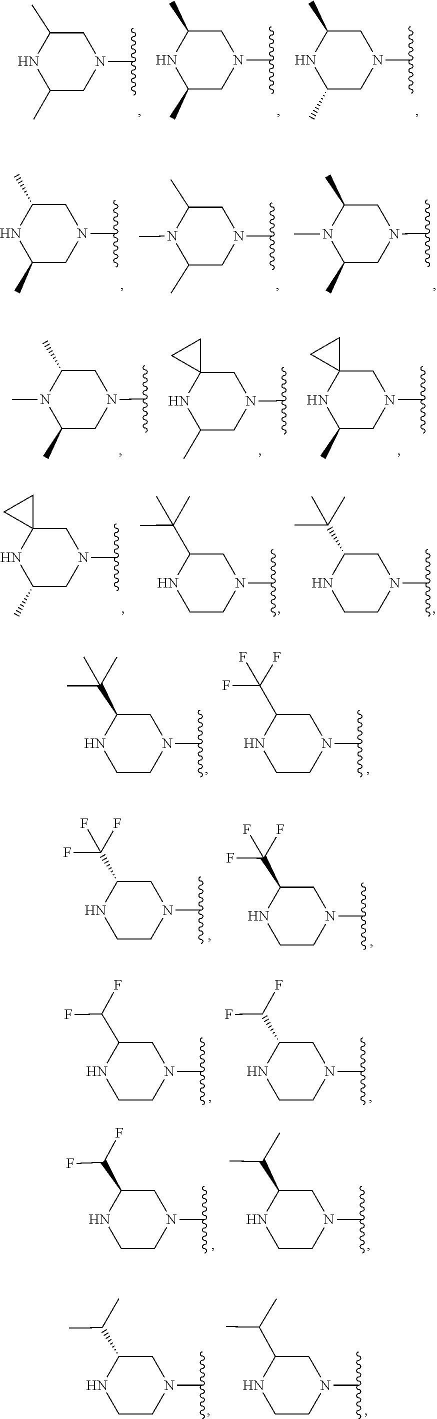

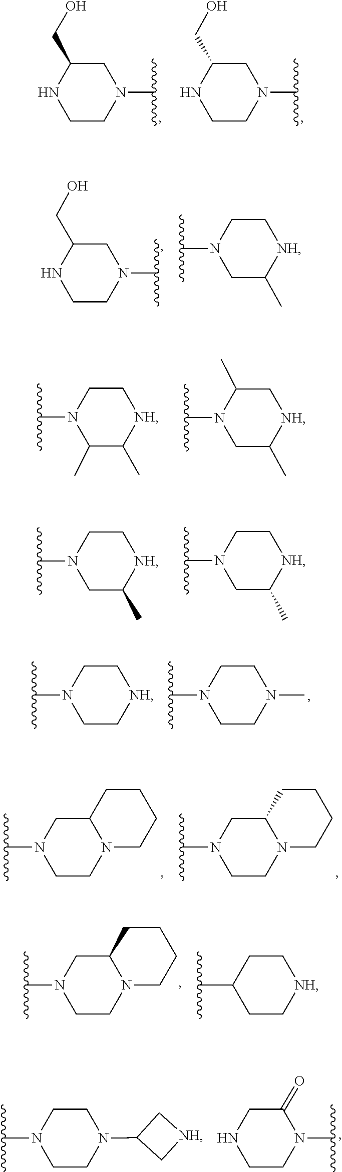

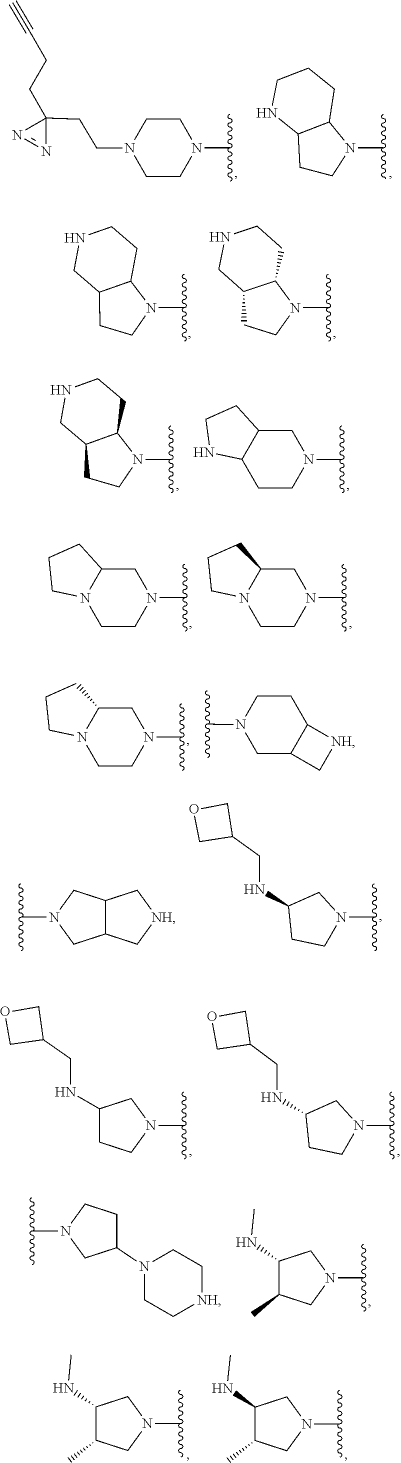

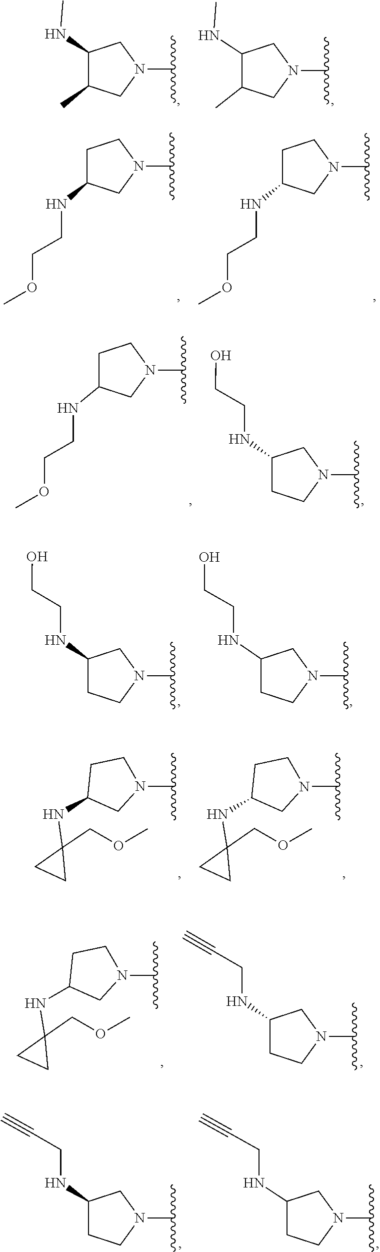

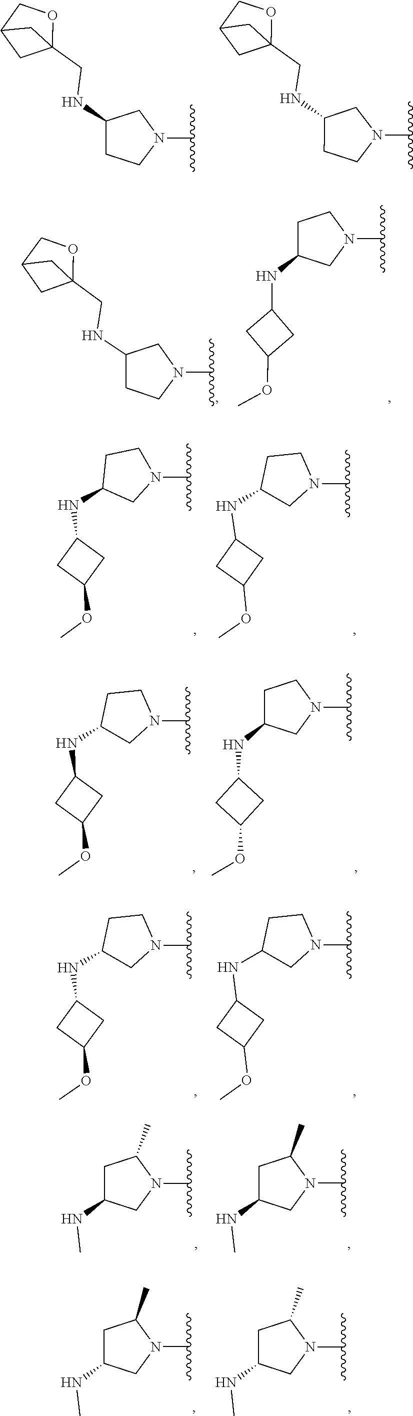

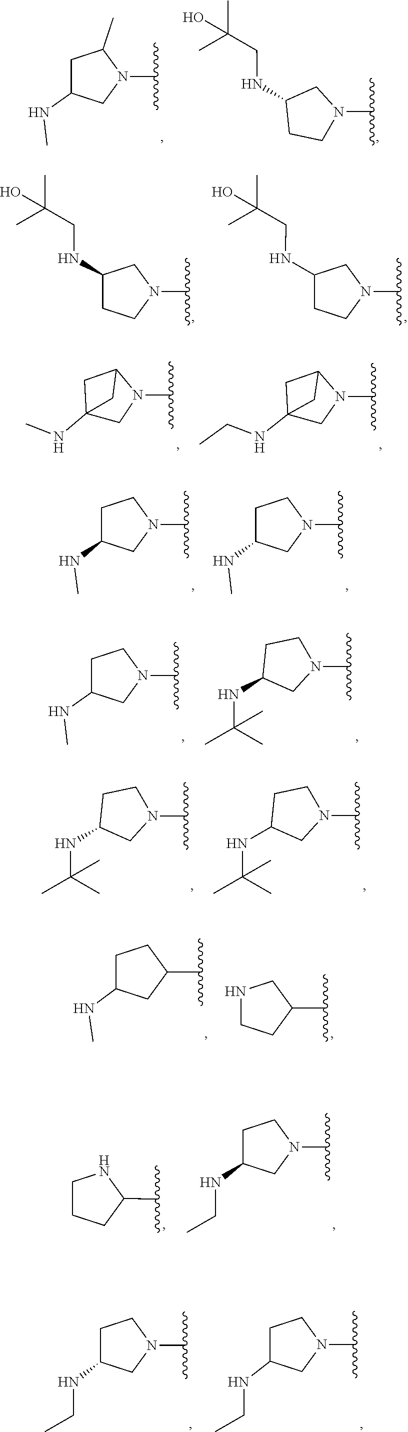

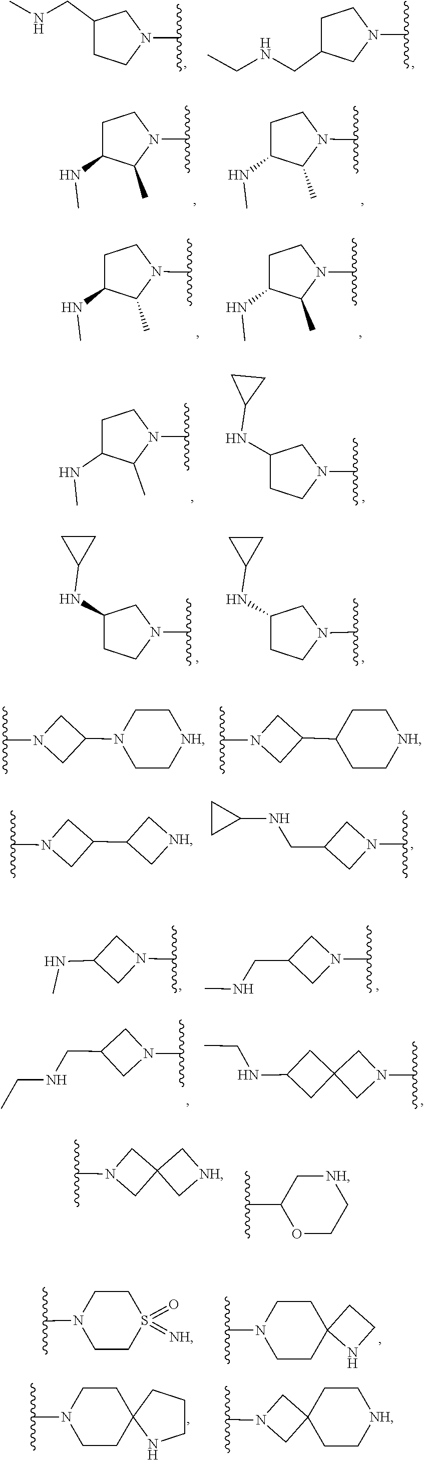

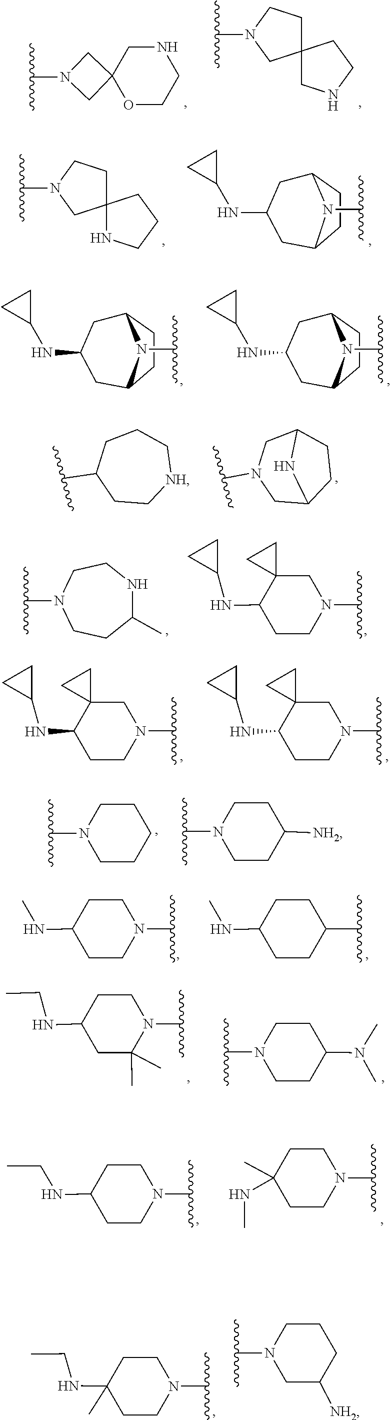

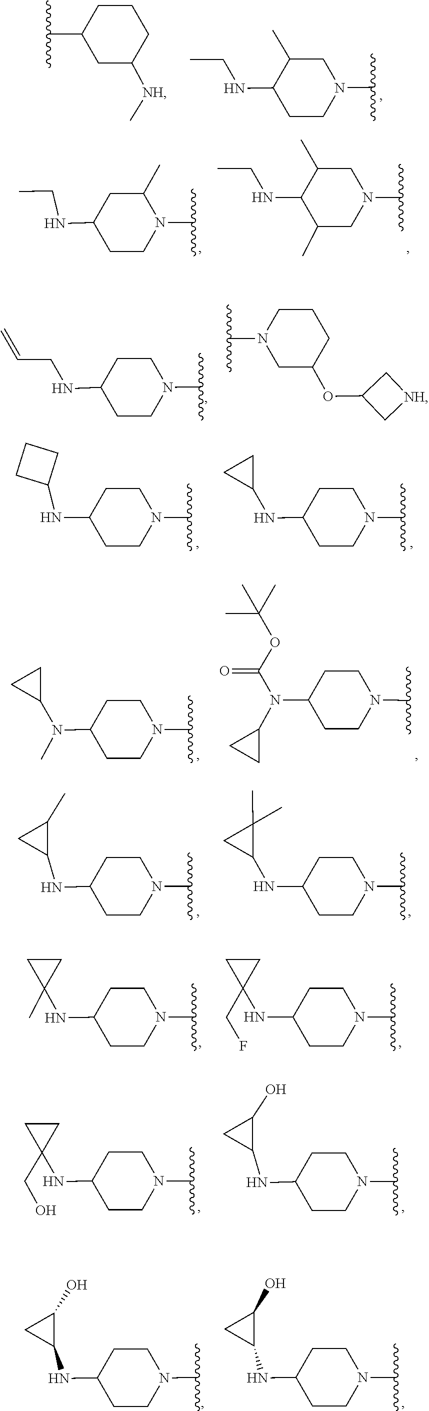

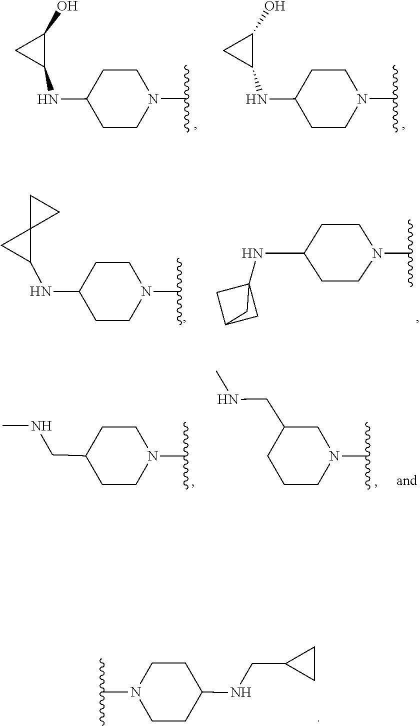

21. The compound of any one of the preceding claims, wherein one of A and B is independently selected from

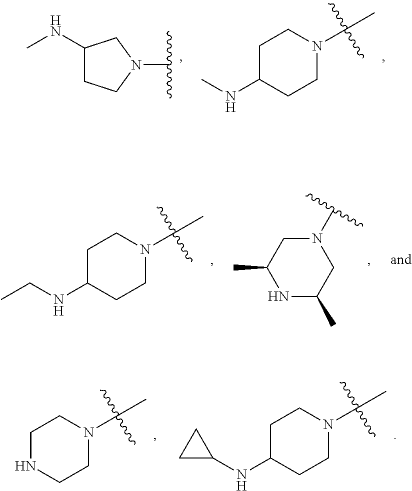

22. The compound of any one of the preceding claims, wherein one of A and B is independently selected from,

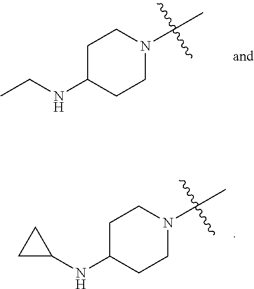

23. The compound of any one of the preceding claims, wherein one of A and B is

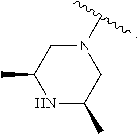

24. The compound of any one of the preceding claims, wherein at least one of X, Y, and Z is N or N(R^{3c}).

25. The compound of any one of the preceding claims, wherein one of X, Y, and Z is C(R^{3a}) (e.g., CH), and the others of X, Y, and Z are each independently N or N(R^{3c}).

26. The compound of any one of the preceding claims, wherein Z and Y are each independently N or N(R^{3c}), and X is C(R^{3a}) (e.g., CH).

27. The compound of any one of the preceding claims, wherein X is C(R^{3a}) (e.g., CH), and Y and Z are each independently N or N(R^{3c}).

28. The compound of any one of the preceding claims, wherein 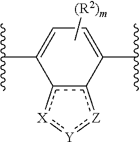
is selected from

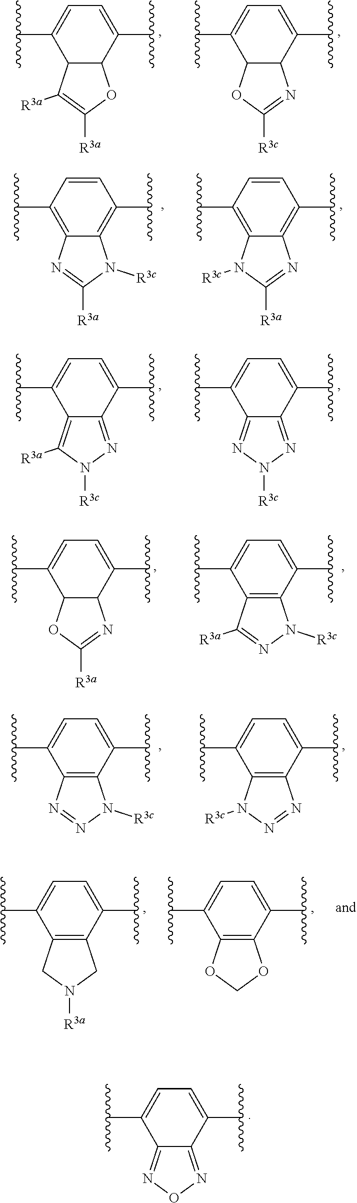

29. The compound of any one of the preceding claims, wherein

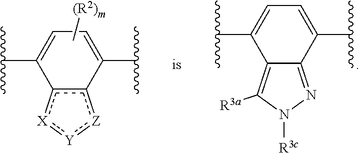

30. The compound of any one of the preceding claims, wherein R^{3c} is C₁-C₆-alkyl, C₂-C₆-alkenyl, C₁-C₆-heteroalkyl, C₁-C₆-haloalkyl, cycloalkyl, heterocyclyl, C₁-C₆ alkylene-heterocyclyl, aryl, C₁-C₆ alkylene-aryl, heteroaryl, C₁-C₆ alkylene-heteroaryl, heteroaryl, wherein each alkyl, alkylene, alkenyl, heteroalkyl, haloalkyl, cycloalkyl, heterocyclyl, aryl, and heteroaryl is optionally substituted with one or more R⁸.

31. The compound of any one of the preceding claims, wherein the compound of Formula (I) is Formula (I-d): 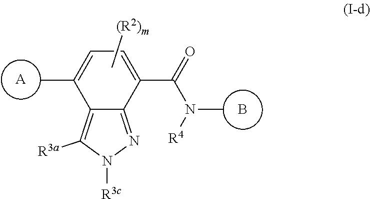
or a pharmaceutically acceptable salt, solvate, hydrate, tautomer, or stereoisomer thereof, wherein A, B, R², R^{3a}, R^{3c}, R⁴, m, and subvariables thereof are as defined in claim 1.

32. The compound of any one of the preceding claims, wherein the compound of Formula (I) is a compound of Formula (I-e): 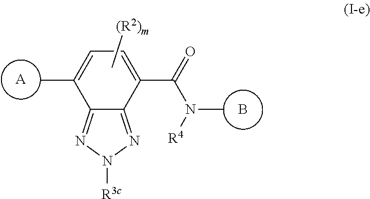
or a pharmaceutically acceptable salt, solvate, hydrate, tautomer, or stereoisomer thereof, wherein each of A, B, R², R^{3c}, R⁴, m, and subvariables thereof are defined as in claim 1.

33. The compound of any one of the preceding claims, wherein the compound of Formula (I) is a compound of Formula (I-f): 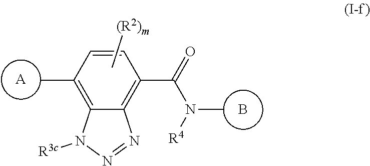
or a pharmaceutically acceptable salt, solvate, hydrate, tautomer, or stereoisomer thereof, wherein each of A, B, R², R^{3c}, R⁴, m, and subvariables thereof are defined as in claim 1.

34. The compound of any one of the preceding claims, wherein the compound of Formula (I) is a compound of Formula (I-g): 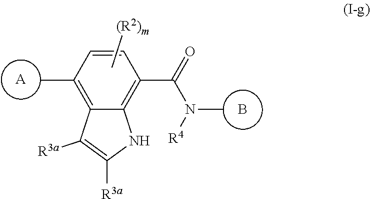
or a pharmaceutically acceptable salt, solvate, hydrate, tautomer, or stereoisomer thereof, wherein each of A, B, R², R^{3a}, R⁴, m, and subvariables thereof are defined as in claim 1.

35. The compound of any one of the preceding claims, wherein the compound of Formula (I) is a compound of Formula (I-h): 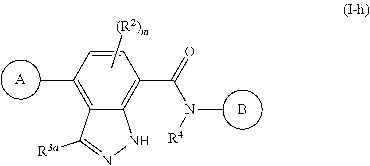
or a pharmaceutically acceptable salt, solvate, hydrate, tautomer, or stereoisomer thereof, wherein each of A, B, R², R^{3a}, R⁴, m, and subvariables thereof are defined as in claim 1.

36. The compound of any one of the preceding claims, wherein the compound of Formula (I) is a compound of Formula (I-i): 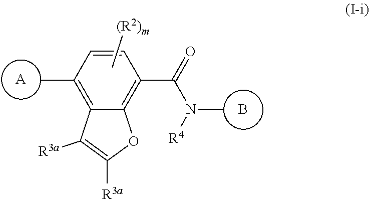
or a pharmaceutically acceptable salt, solvate, hydrate, tautomer, or stereoisomer thereof, wherein each of A, B, R², R^{3a}, R⁴, m, and subvariables thereof are defined as in claim 1.

37. The compound of any one of the preceding claims, wherein the compound of Formula (I) is a compound of Formula (I-j): 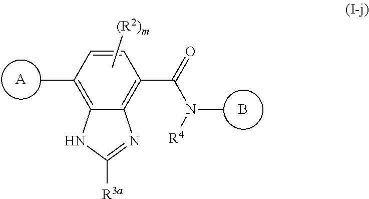
or a pharmaceutically acceptable salt, solvate, hydrate, tautomer, or stereoisomer thereof, wherein each of A, B, R², R^{3a}, R⁴, m, and subvariables thereof are defined as in claim 1.

38. The compound of any one of the preceding claims, wherein the compound of Formula (I) is a compound of Formula (I-k): 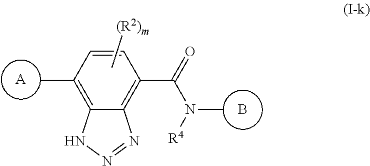
or a pharmaceutically acceptable salt, solvate, hydrate, tautomer, or stereoisomer thereof, wherein each of A, B, R², R^{3c}, R⁴, m, and subvariables thereof are defined as in claim 1.

39. The compound of any one of the preceding claims, wherein the compound of Formula (I) is a compound of Formula (I-1): 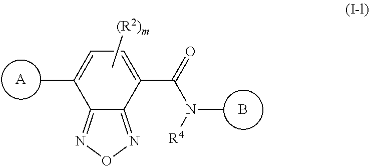
or a pharmaceutically acceptable salt, solvate, hydrate, tautomer, or stereoisomer thereof, wherein each of A, B, R², R⁴, m, and subvariables thereof are defined as in claim 1.

40. The compound of any one of the preceding claims, wherein the compound of Formula (I) is a compound of Formula (I-m): 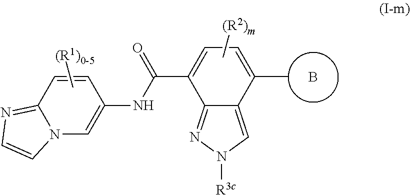
or a pharmaceutically acceptable salt, solvate, hydrate, tautomer, or stereoisomer thereof, wherein each of B, R¹, R², R^{3c}, m, and subvariables thereof are defined as in claim 1.

41. The compound of any one of the preceding claims, wherein the compound of Formula (I) is a compound of Formula (I-n): 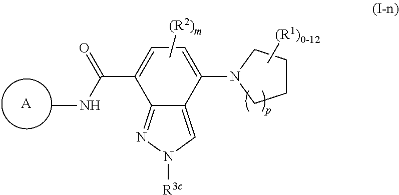
or a pharmaceutically acceptable salt, solvate, hydrate, tautomer, or stereoisomer thereof, wherein p is 0, 1, 2, or 3, and each of A, R¹, R², R^{3c}, m, and subvariables thereof are defined as in claim 1.

42. A compound of Formula (II-c): 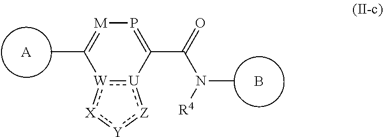
or a pharmaceutically acceptable salt, solvate, hydrate, tautomer, or stereoisomer thereof, wherein:
A is a nitrogen-containing heterocyclyl optionally substituted with one or more R¹;
B is a nitrogen-containing heteroaryl and optionally substituted with one or more R¹;
M and P are each independently C(R²) or N;
U and W are each independently C or N;
X, Y, and Z are each independently C(R^{3a}), C(R^{3a})(R^{3b}), N, N(R^{3c}), S, or O, wherein at least one of X, Y, and Z is N or N(R^{3c}), and the bonds in the ring comprising U, W, X, Y, and Z may be single or double bonds as valency permits;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶;
each R² is independently hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A},
R^{3a} and R^{3b} are each independently hydrogen, C₁-C₆-alkyl, C₂-C₆-alkenyl, C₂-C₆-alkynyl, C₁-C₆-heteroalkyl, C₁-C₆-haloalkyl, halo, cyano, OR^{A}, —NR^{B}R^{C}, —C(O)R^{D}, or —C(O)OR^{D}; or
each of R^{3a} and R^{3b}, together with the carbon atom to which they are attached, form an oxo group;
R^{3c} is hydrogen, C₁-C₆-alkyl, C₂-C₆-alkenyl, C₂-C₆-alkynyl, C₁-C₆-heteroalkyl, C₁-C₆-haloalkyl, cycloalkyl, C₁-C₆ alkylene-cycloalkyl, heterocyclyl, C₁-C₆ alkylene-heterocyclyl, aryl, C₁-C₆ alkylene-aryl, heteroaryl, C₁-C₆ alkylene-heteroaryl, or —C(O)R^{D}, wherein each alkyl, alkylene, alkenyl, alkynyl, heteroalkyl, haloalkyl, cycloalkyl, heterocyclyl, aryl, and heteroaryl is optionally substituted with one or more R⁸;
each R⁴ is independently hydrogen, C₁-C₆-alkyl, C₁-C₆-heteroalkyl, or C₁-C₆-haloalkyl;
each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —NR^{B}N(R^{B})(R^{C}), —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R¹¹;
each R^{A} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}, wherein C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, and C₁-C₆ alkylene-heteroaryl is optionally substituted with one or more R⁷;
each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, —OR^{A}, —C(O)NR^{B}R^{C}, —C(O)R^{D}, —C(O)OR^{D}, or —S(O)ₓR^{D}, wherein C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, and C₁-C₆ alkylene-heteroaryl is optionally substituted with one or more R⁷; or
R^{B} and R^{C} together with the atom to which they are attached form a 3-7-membered heterocyclyl ring optionally substituted with one or more R⁷;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R⁷ is C₁-C₆-alkyl, halo, cyano, oxo, or —OR^{A1};
each R¹¹ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A1} is hydrogen or C₁-C₆-alkyl; and
x is 0, 1, or 2.

43. The compound of any one of the preceding claims, wherein B is monocyclic heteroaryl optionally substituted with one or more R¹.

44. The compound of any one of the preceding claims, wherein B is bicyclic heteroaryl optionally substituted with one or more R¹.

45. The compound of any one of the preceding claims, wherein B is a 5-10 membered heteroaryl optionally substituted with one or more R¹.

46. The compound of any one of the preceding claims, wherein B is a bicyclic nitrogen-containing heteroaryl containing at least two nitrogen atoms optionally substituted with one or more R¹.

47. The compound of any one of the preceding claims, wherein B is selected from 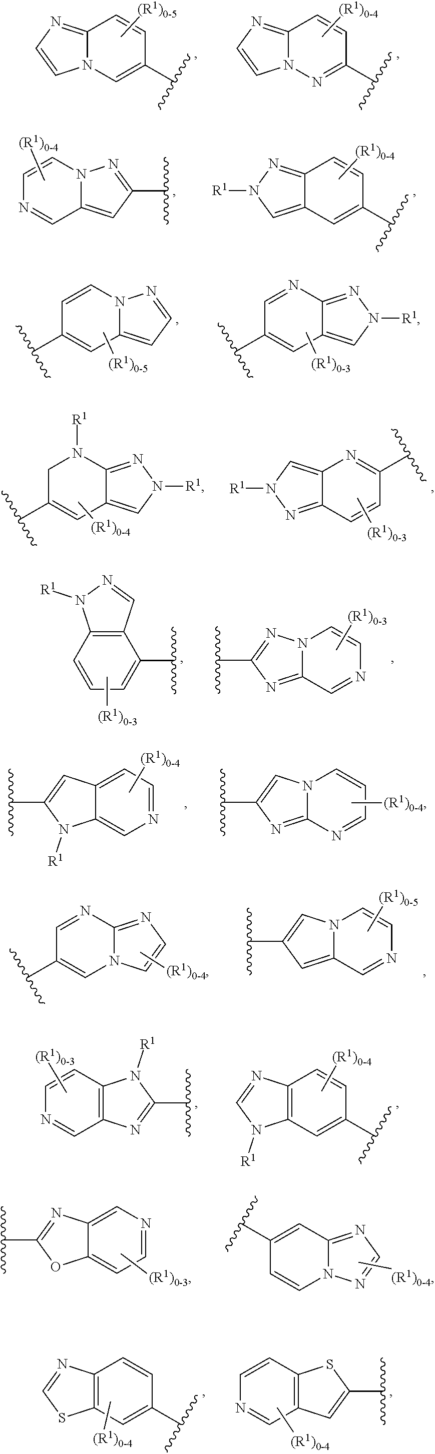 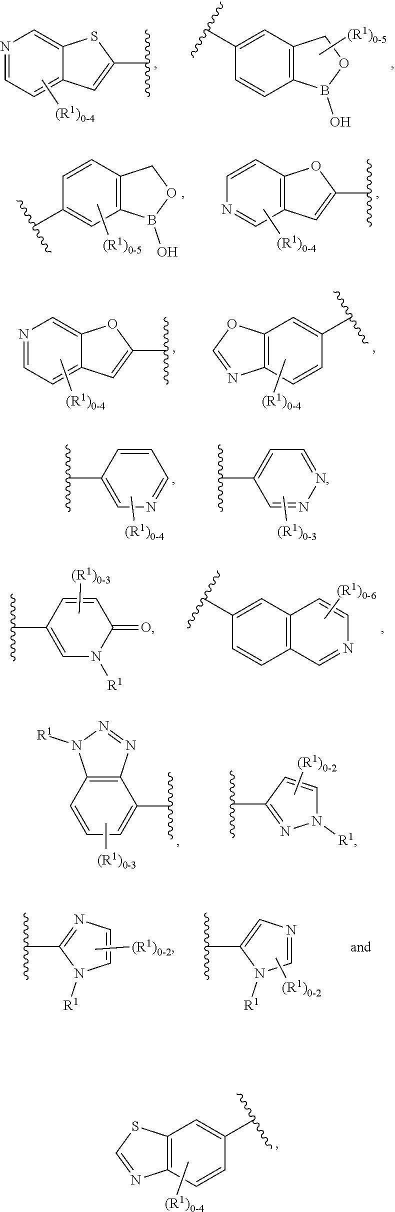
wherein R¹ is as described in claim 42.

48. The compound of any one of the preceding claims, wherein B is selected from 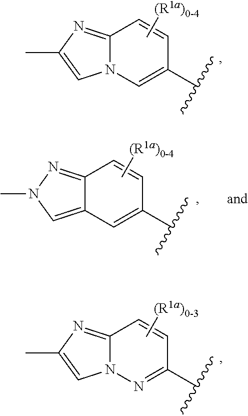
and each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

49. The compound of any one of the preceding claims, wherein B is 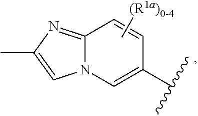
and each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

50. The compound of any one of the preceding claims, wherein B is 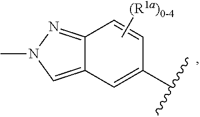
and each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

51. The compound of any one of the preceding claims, wherein B is selected from

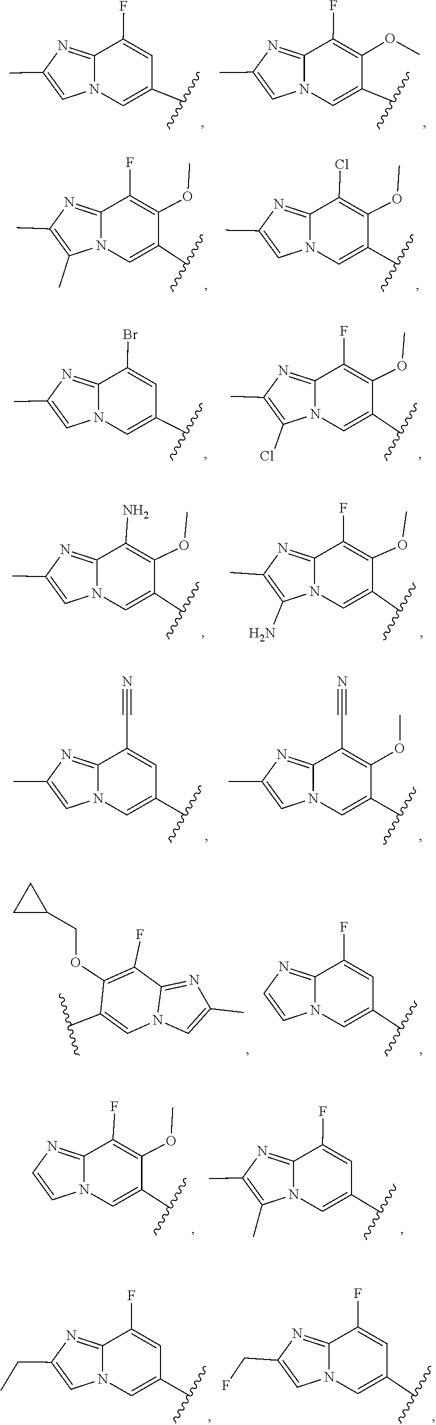

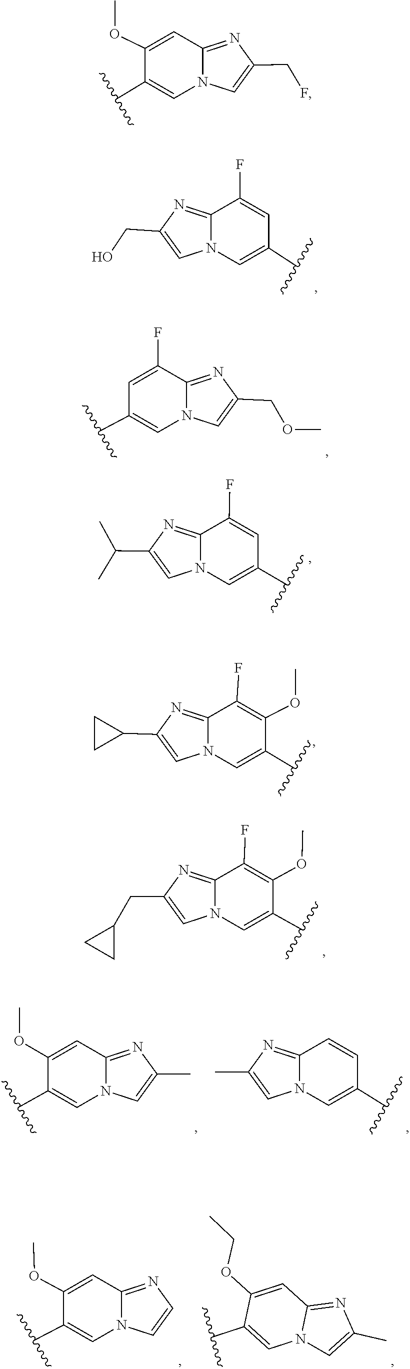

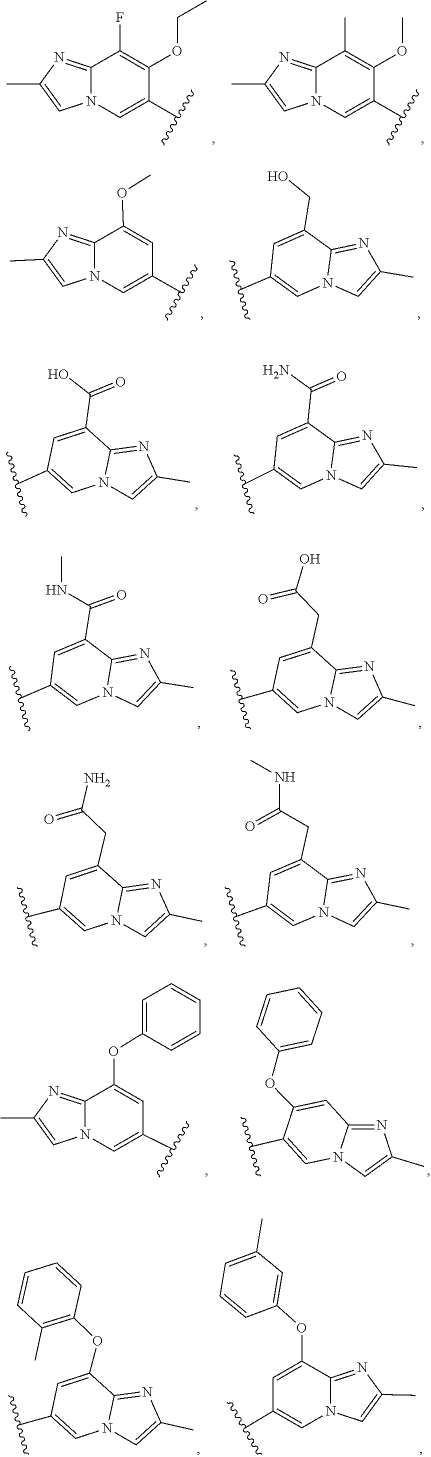

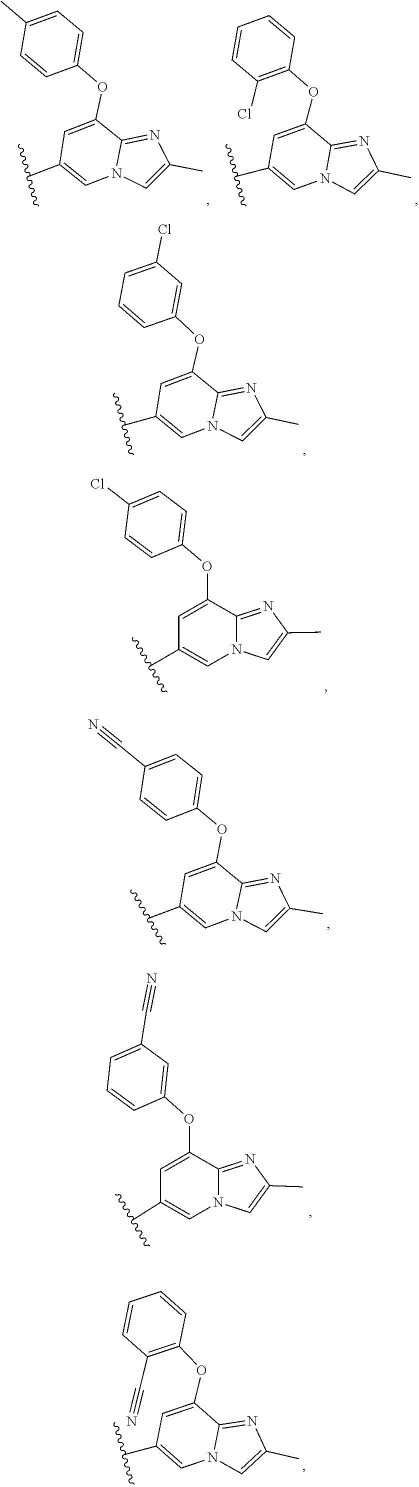

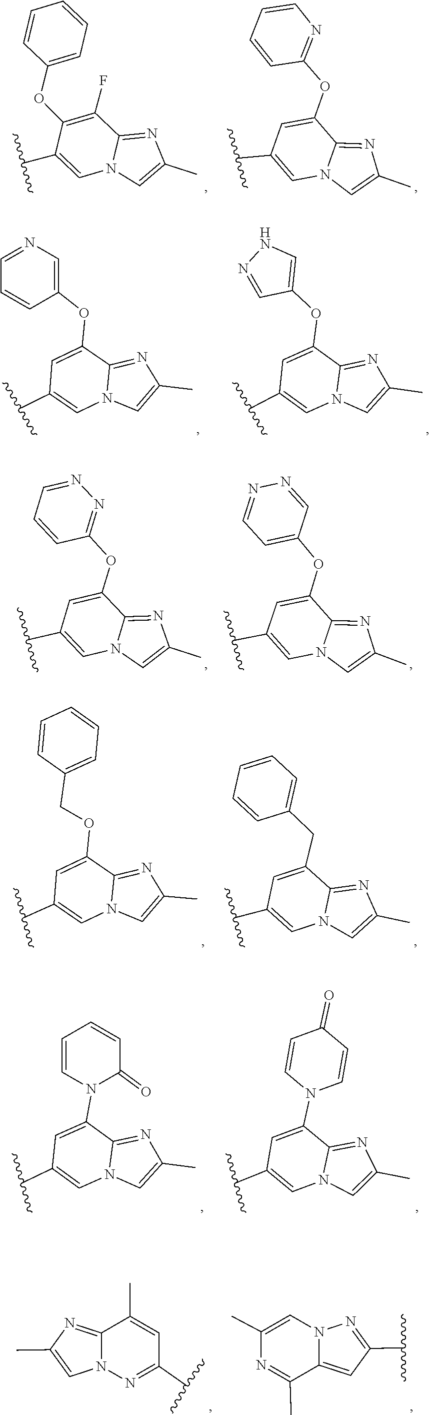

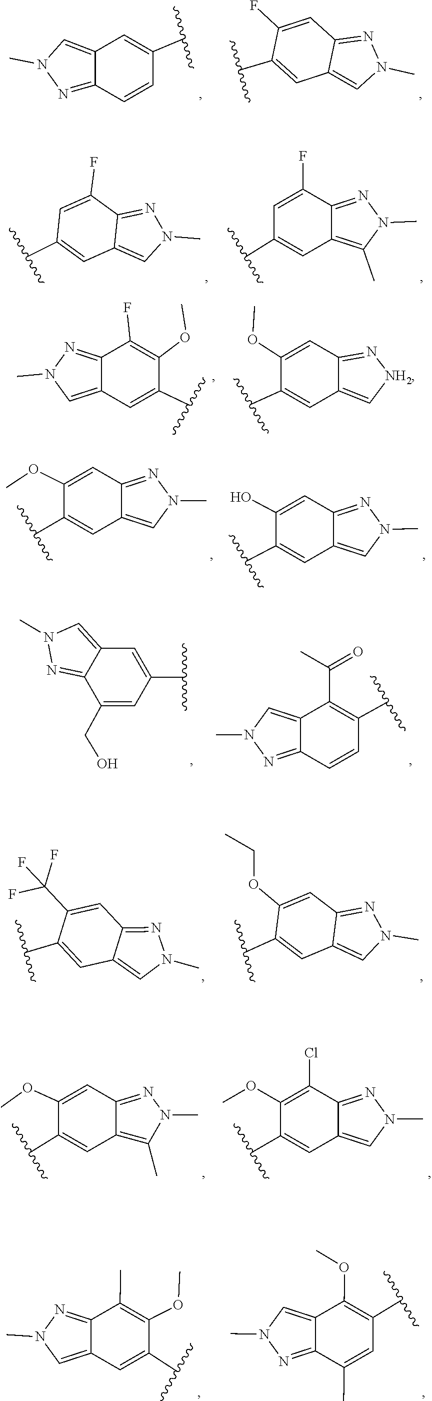

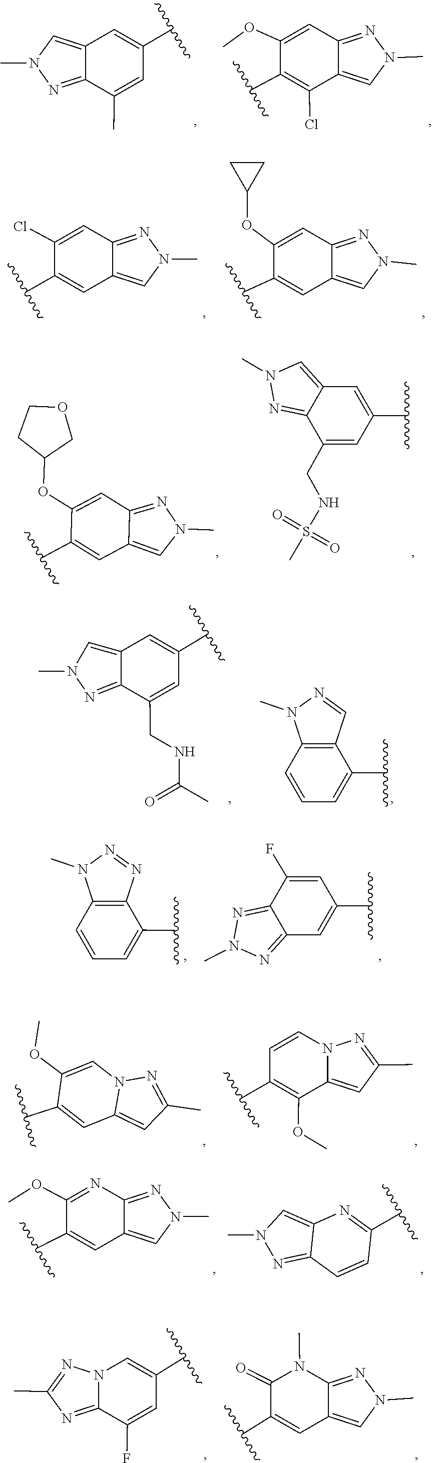

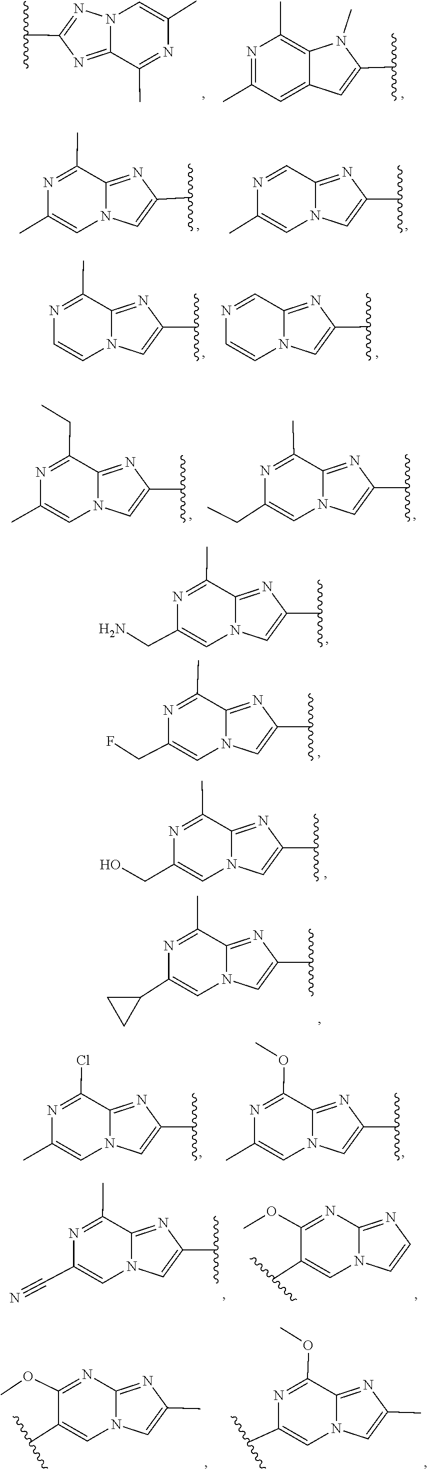

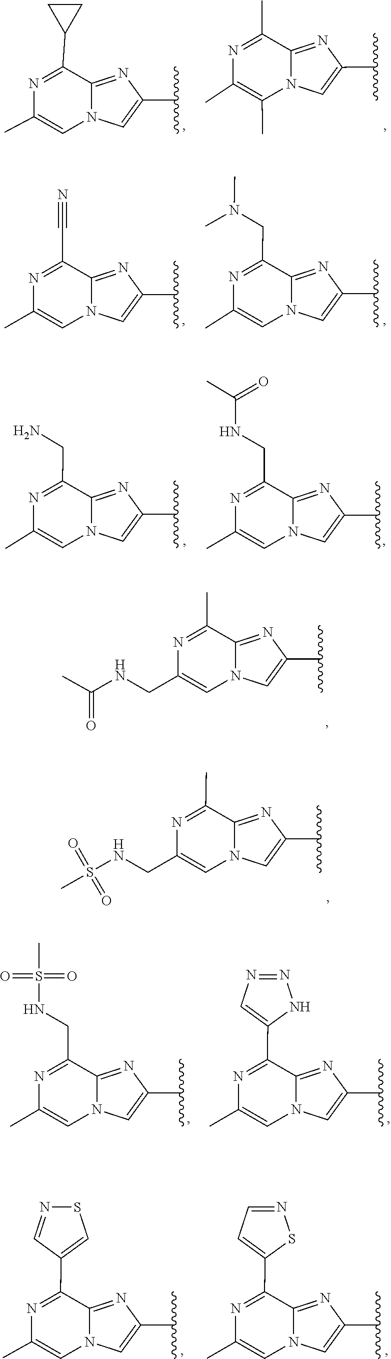

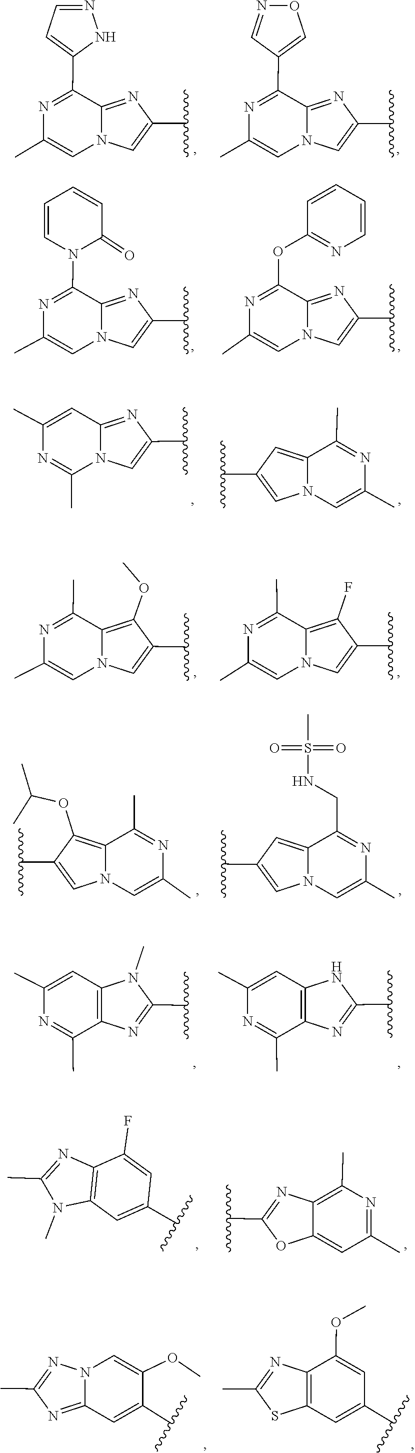

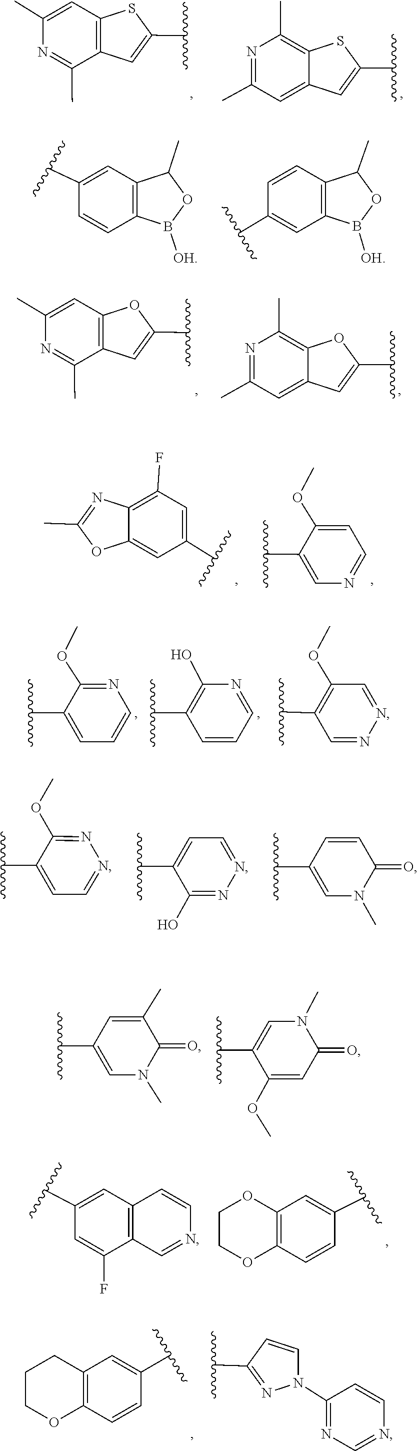

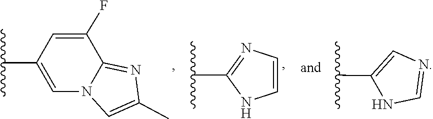 [SMILES: *c1cc(F)c2nc(C)cn2c1 ; *c1ncc[nH]1 ; *c1cnc[nH]1]

52. The compound of any one of the preceding claims, wherein B is selected from

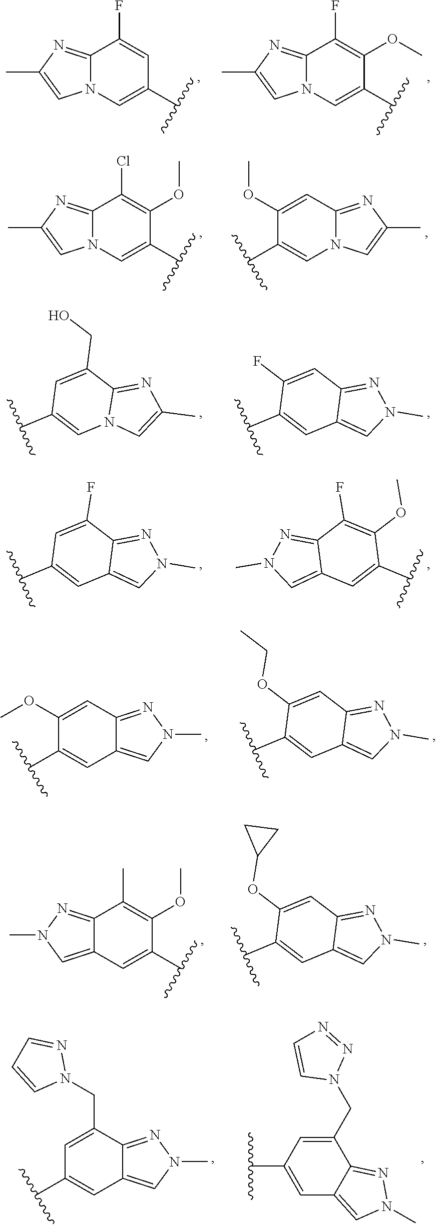

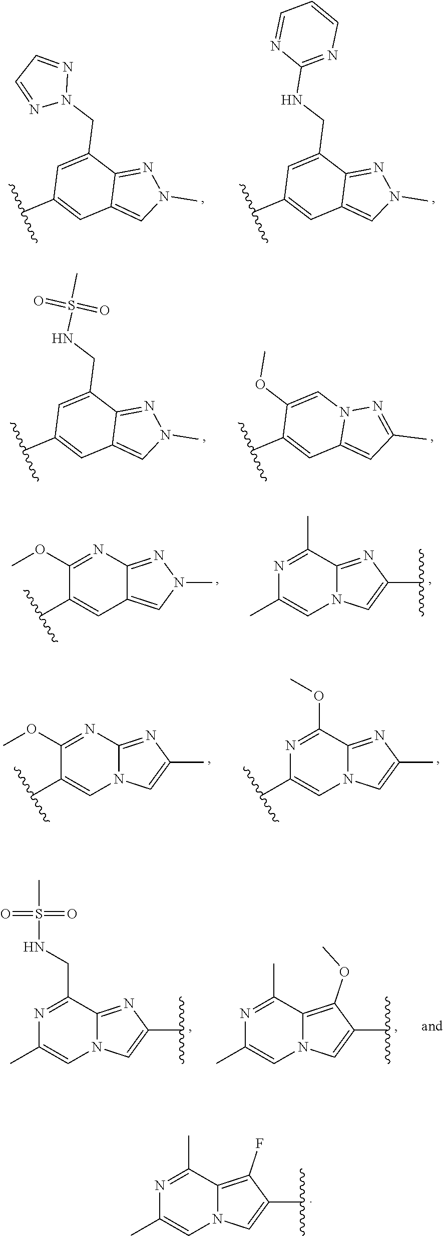

53. The compound of any one of the preceding claims, wherein B is

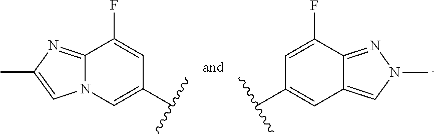 [SMILES: *c1cc(F)c2nc(C)cn2c1 ; *c1cc(F)c2nn(C)cc2c1]

54. The compound of any one of the preceding claims, wherein A is monocyclic heterocyclyl or bicyclic heterocyclyl, each of which is optionally substituted with one or more R¹.

55. The compound of any one of the preceding claims, wherein A is a monocyclic nitrogen-containing heterocyclyl optionally substituted with one or more R¹.

56. The compound of any one of the preceding claims, wherein A is a 4-8 membered heterocyclyl optionally substituted with one or more R¹.

57. The compound of any one of the preceding claims, wherein A is selected from 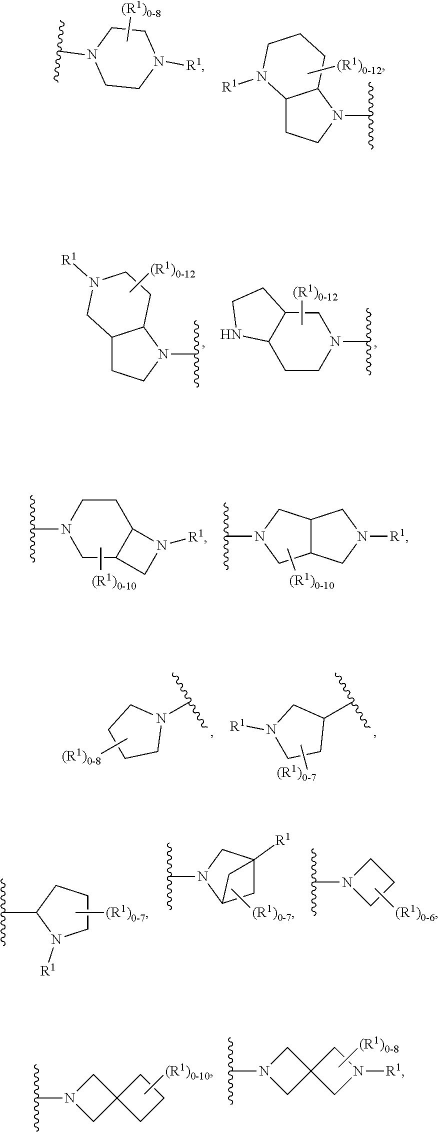 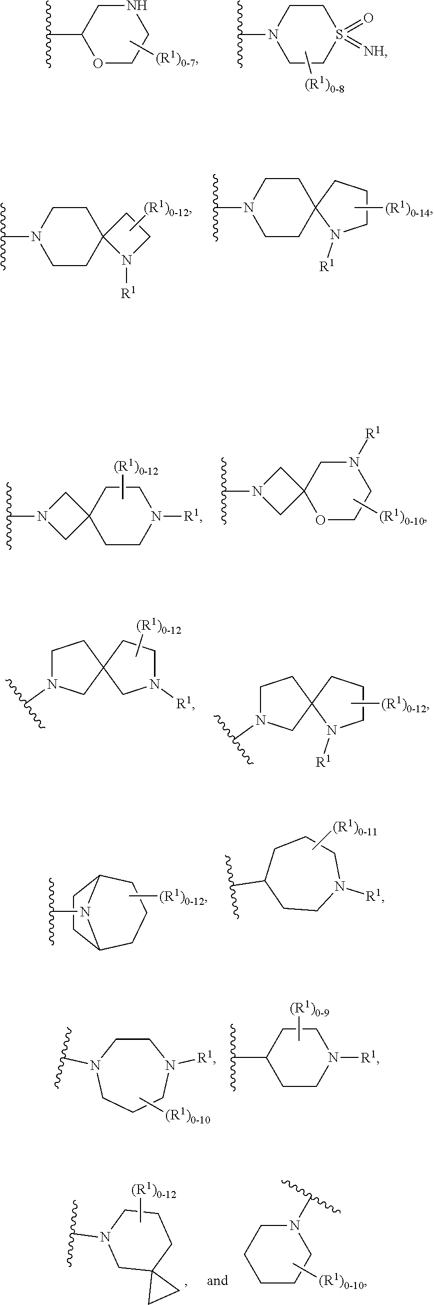
and R¹ is as described in claim 1.

58. The compound of any one of the preceding claims, wherein A is selected from 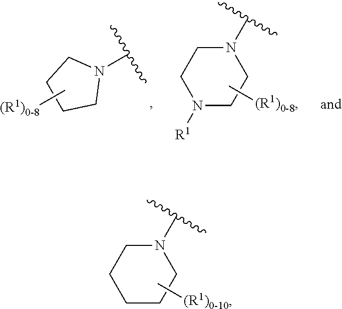
and R¹ is as described in claim 1.

59. The compound of any one of the preceding claims, wherein A is 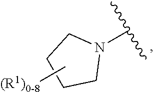
and R¹ is as described in claims 42

60. The compound of any one of the preceding claims, wherein A is selected from

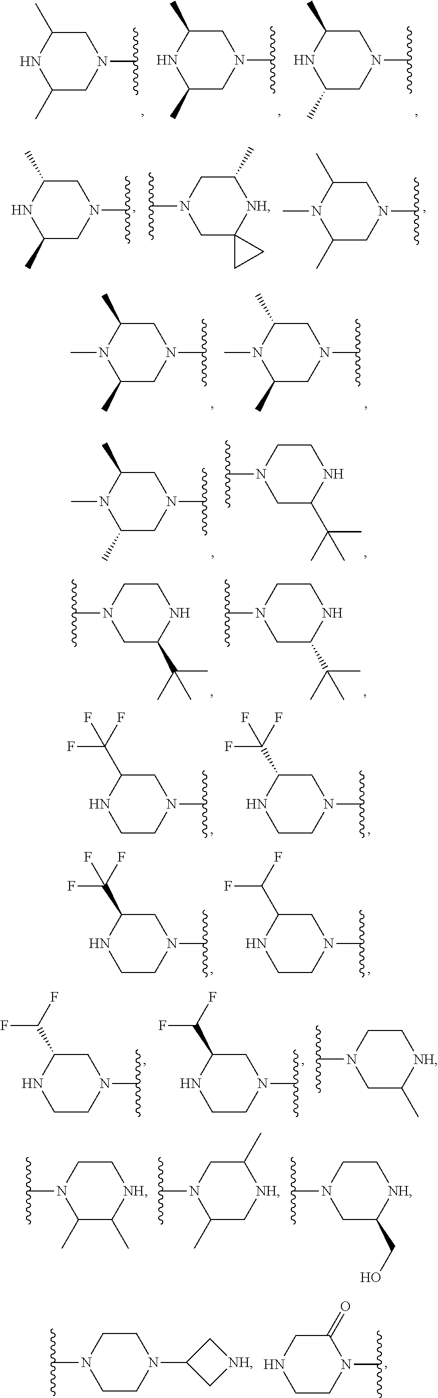

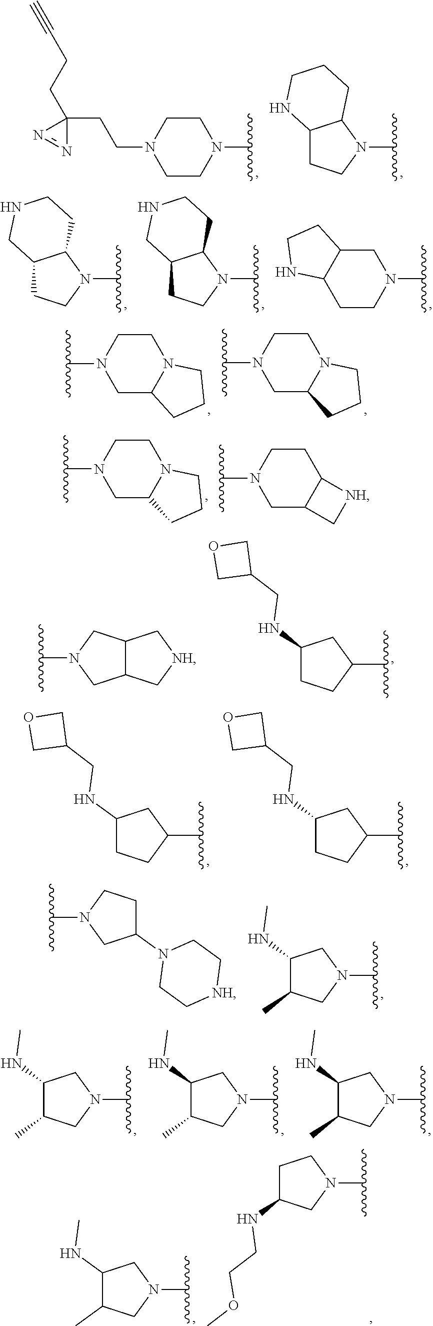

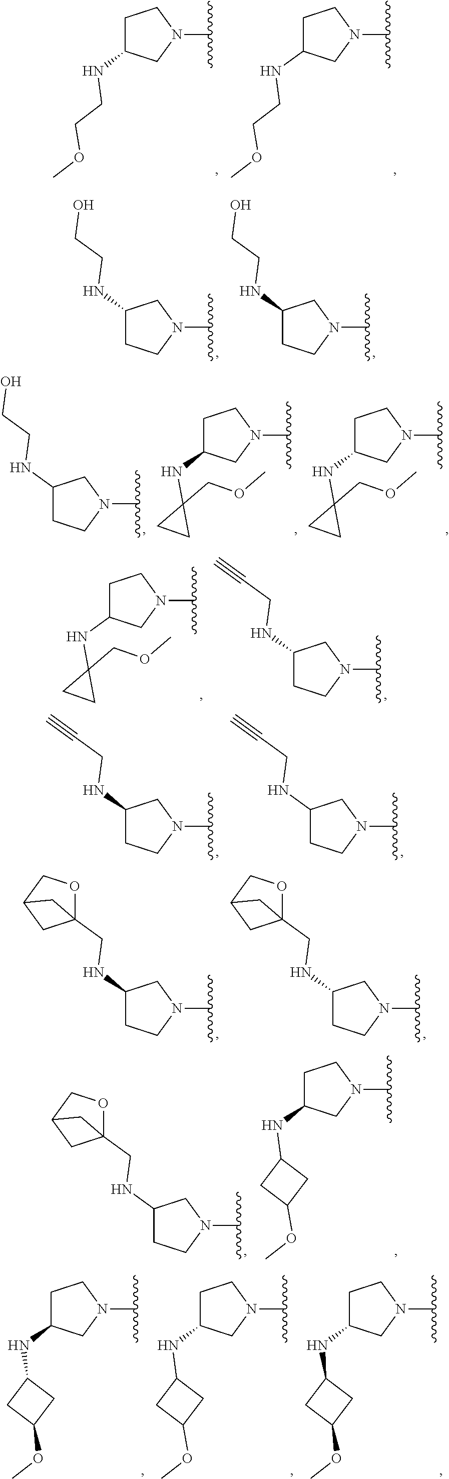

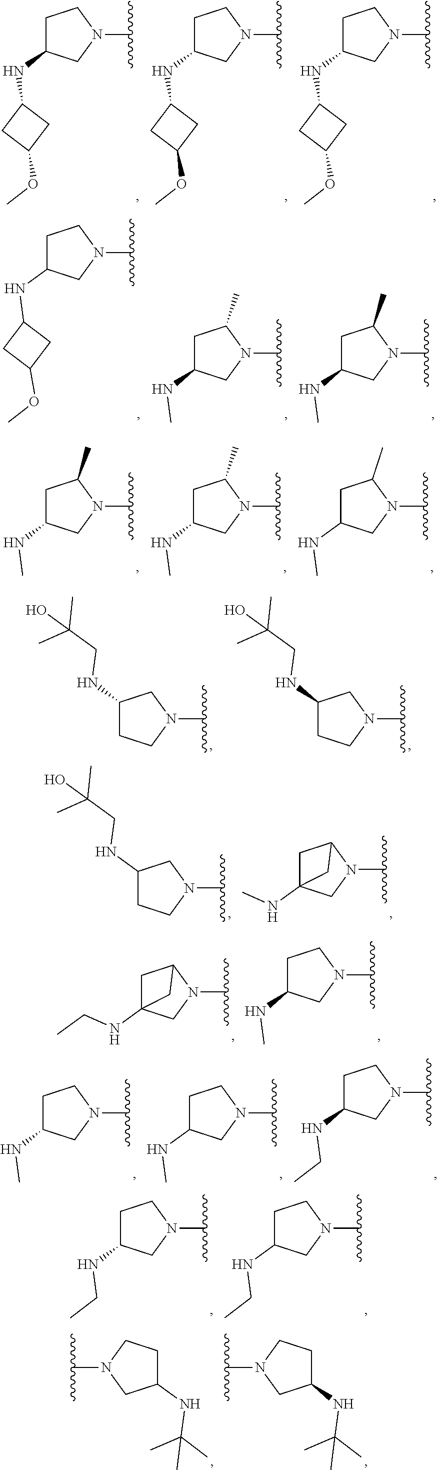

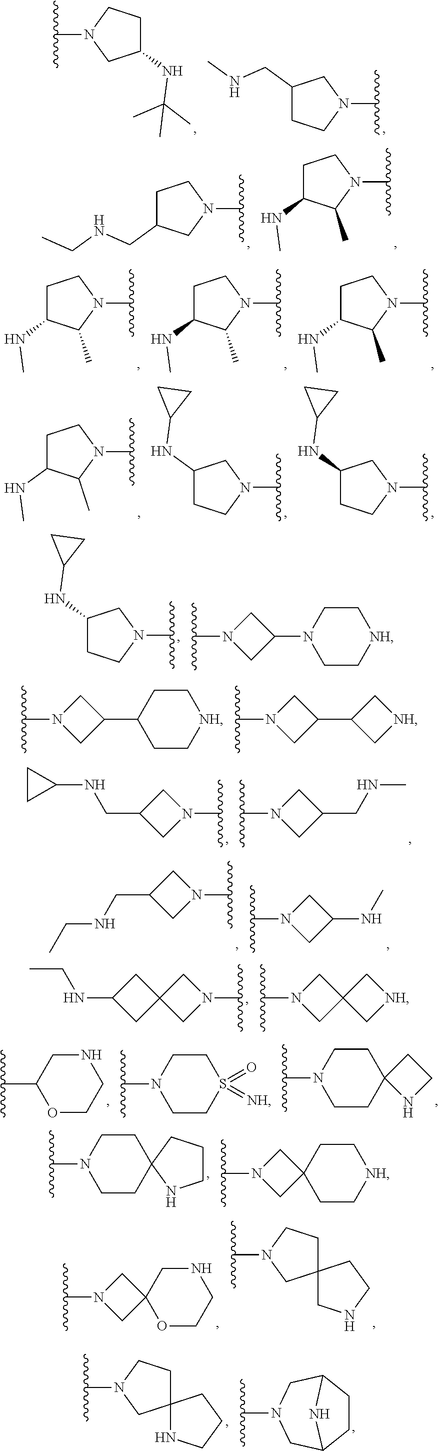

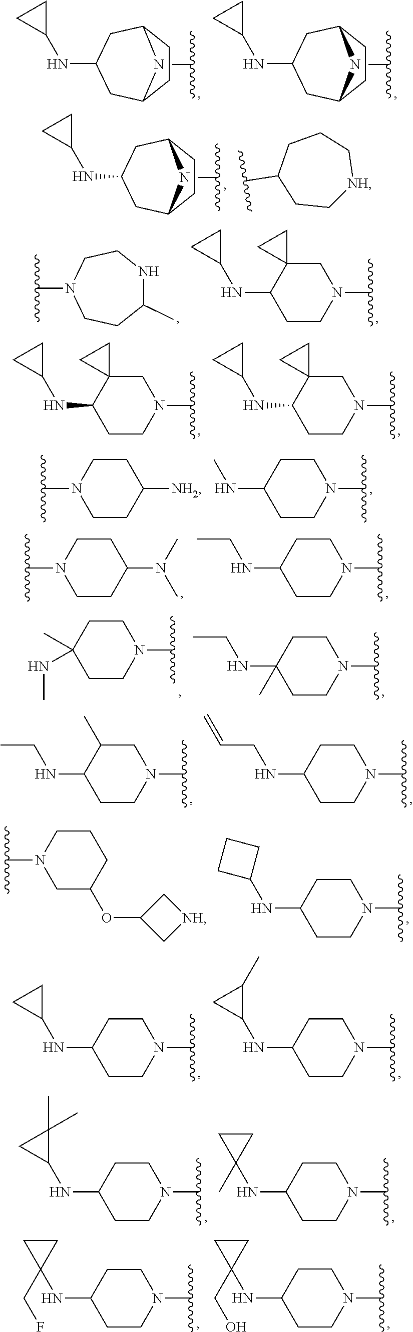

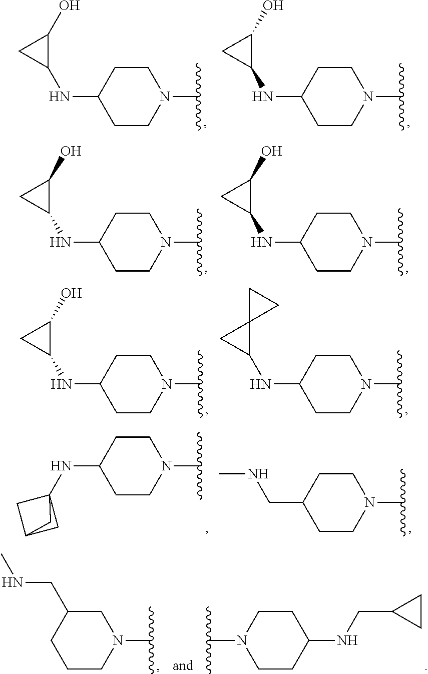

61. The compound of any one of the preceding claims, wherein one of A and B is independently selected from

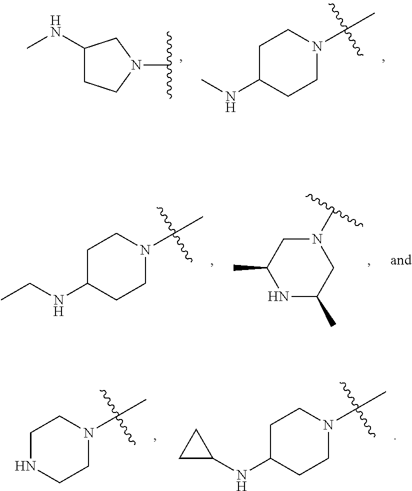

62. The compound of any one of the preceding claims, wherein one of A and B is independently selected from,

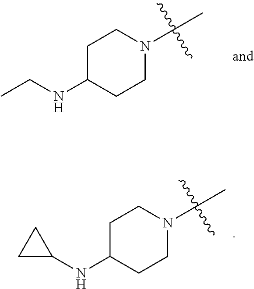

63. The compound of any one of the preceding claims, wherein one of A and B is

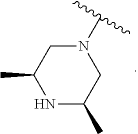

64. The compound of any one of the preceding claims, wherein each of M and P is independently C(R²) (e.g., CH).

65. The compound of any one of the preceding claims, wherein M is C(R²) (e.g., CH) and P is N.

66. The compound of any one of the preceding claims, wherein M is N and P is C(R²) (e.g., CH).

67. The compound of any one of the preceding claims, wherein each of U and W is independently C.

68. The compound of any one of the preceding claims, wherein U is C and W is N.

69. The compound of any one of the preceding claims, wherein U is N and W is C.

70. The compound of any one of the preceding claims, wherein one of X, Y, and Z is C(R^{3a}) (e.g., CH), and the others of X, Y, and Z are each independently N, N(R^{3c}), or S.

71. The compound of any one of the preceding claims, wherein two of X, Y, and Z are independently N or N(R^{3c}).

72. The compound of any one of the preceding claims, wherein X and Y are each independently N or N(R^{3c}), and Z is C(R^{3a}) (e.g., CH).

73. The compound of any one of the preceding claims, wherein X is C(R^{3a}) (e.g., CH), and Y and Z are each independently N or N(R^{3c}).

74. The compound of any one of the preceding claims, wherein two of X, Y, and Z independently is C(R^{3a}) (e.g., CH).

75. The compound of any one of the preceding claims, wherein 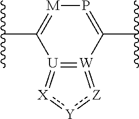
is selected from

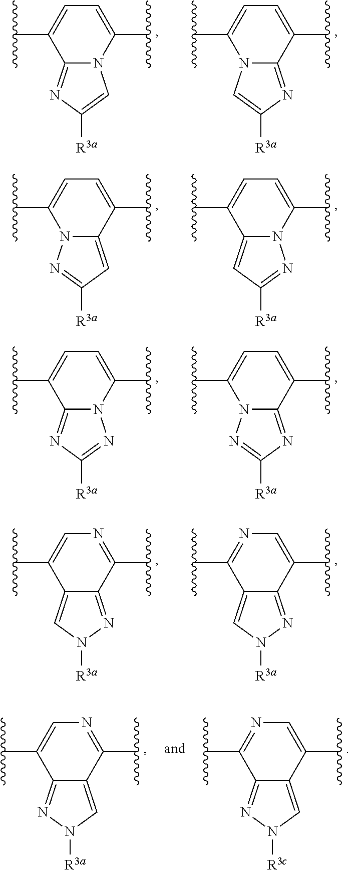

76. The compound of any one of the preceding claims, wherein the compound of Formula (II) is Formula (II-d): 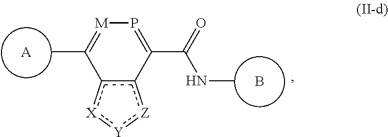
or a pharmaceutically acceptable salt, solvate, hydrate, tautomer, or stereoisomer thereof, wherein each of A, B, M, P, X, Y, Z, and subvariables thereof are defined as in claim 42.

77. The compound of any one of the preceding claims, wherein the compound of Formula (II) is Formula (II-e): 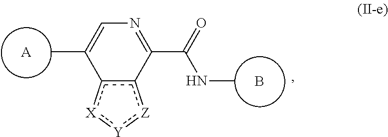
or a pharmaceutically acceptable salt, solvate, hydrate, tautomer, or stereoisomer thereof, wherein each of A, B, X, Y, Z, and subvariables thereof are defined as in claim 42.

78. The compound of any one of the preceding claims, wherein the compound of Formula (II) is Formula (II-f): 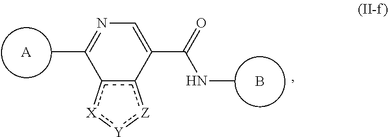
or a pharmaceutically acceptable salt, solvate, hydrate, tautomer, or stereoisomer thereof, wherein each of A, B, X, Y, Z, and subvariables thereof are defined as in claim 42.

79. The compound of any one of the preceding claims, wherein the compound of Formula (II) is Formula (II-g): 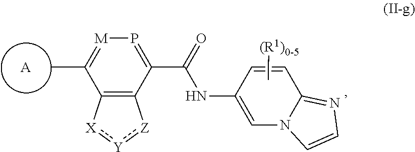
or a pharmaceutically acceptable salt, solvate, hydrate, tautomer, or stereoisomer thereof, wherein each of B, M, P, X, Y, Z, R¹, and subvariables thereof are defined as in claim 42.

80. The compound of any one of the preceding claims, wherein the compound of Formula (II) is Formula (II-h): 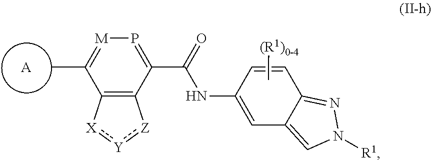
or a pharmaceutically acceptable salt, solvate, hydrate, tautomer, or stereoisomer thereof, wherein each of B, M, P, X, Y, Z, R¹, and subvariables thereof are defined as in claim 39.

81. The compound of any one of the preceding claims, wherein the compound is selected from any one of the compounds shown in Table 1 or a pharmaceutically acceptable salt, solvate, hydrate, tautomer, or stereoisomer thereof.

82. A pharmaceutical composition comprising a compound of any one of the preceding claims and a pharmaceutically acceptable excipient.

83. The compound of any one of claims 1-81, or the pharmaceutical composition of claim 82, wherein the compound alters a target nucleic acid (e.g., an RNA, e.g., a pre-mRNA).

84. The compound of any one of claims 1-81, or the pharmaceutical composition of claim 82, wherein the compound binds to a target nucleic acid (e.g., an RNA, e.g., a pre-mRNA).

85. The compound of any one of claims 1-81, or the pharmaceutical composition of claim 82, wherein the compound stabilizes a target nucleic acid (e.g., an RNA, e.g., a pre-mRNA).

86. The compound of any one of claims 1-81, or the pharmaceutical composition of claim 82, wherein the compound increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR.

87. The compound of any one of claims 1-81, or the pharmaceutical composition of claim 82, wherein the compound decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR %.

88. A method of forming a complex comprising a component of a spliceosome (e.g., a major spliceosome component or a minor spliceosome component), a nucleic acid (e.g., a DNA, RNA, e.g., a pre-mRNA), and a compound of Formula (I) or (II) or a composition thereof according to any one of claims 1-81:
comprising contacting the nucleic acid (e.g., a DNA, RNA, e.g., a pre-mRNA) with a compound of Formula (I) or (II).

89. The method of claim 88, wherein the component of a spliceosome is recruited to the nucleic acid in the presence of the compound of Formula (I) or (II).

90. A method of altering the conformation of a nucleic acid (e.g., a DNA, RNA, e.g., a pre-mRNA) comprising contacting the nucleic acid with a compound of Formula (I) or (II) according to any one of claims 1-81, or the pharmaceutical composition of claim 82.

91. The method of claim 90, wherein the altering comprises forming a bulge in the nucleic acid.

92. The method of claim 90, wherein the altering comprises stabilizing a bulge in the nucleic acid.

93. The method of claim 90, wherein the altering comprises reducing a bulge in the nucleic acid.

94. The method of any one of any one of claims 90-93, wherein the nucleic acid comprises a splice site.

95. A method for treating a disease or disorder in a subject comprising administering to the subject a compound of Formula (I) or (II) according to any one of claims 1-81 or the pharmaceutical composition of claim 82.

96. The method of claim 95, wherein the disease or disorder comprises a proliferative disease (e.g., cancer, a benign neoplasm, or angiogenesis).

97. The method of any one of claims 95-96, wherein the proliferative disease is cancer.

98. The method of any one of claims 96-97, wherein the proliferative disease comprises adenoid cystic carcinoma, colorectal cancer, leukemia, lung cancer, prostate cancer, breast cancer, or ovarian cancer.

99. The method of claim 95, wherein the disease or disorder comprises a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

100. The method of claim 99, wherein the disease or disorder comprises a neurological disease or disorder.

101. The method of claim 100, wherein the disease or disorder comprises Huntington's disease.
